(19) United States
(12) Patent Application Publication
(10) Pub. No.: US 2025/0242099 A1
(43) Pub. Date: Jul. 31, 2025

(54) VASCULAR VALVES AND SERVOVALVES - AND PROSTHETIC DISORDER RESPONSE SYSTEMS

(71) Applicant: David S. Goldsmith, Atlanta, GA (US)

(72) Inventor: David S. Goldsmith, Atlanta, GA (US)

(21) Appl. No.: 19/033,348

(22) Filed: Jan. 21, 2025

Related U.S. Application Data
(63) Continuation-in-part of application No. 16/873,914, filed on Aug. 11, 2020, now abandoned.

(51) Int. Cl. A61M 1/36; A61M 39/02; A61M 39/22; G16H 40/63

(52) U.S. Cl. CPC A61M 1/36; A61M 39/0247; A61M 39/223; G16H 40/63; A61M 2039/0202; A61M 2039/0205; A61M 2039/0258; A61M 2202/0007; A61M 2202/04; A61M 2205/0272; A61M 2205/10; A61M 2205/3303; A61M 2205/3327; A61M 2205/3331; A61M 2205/50; A61M 2210/12; A61M 2230/005; A61M 2240/00

(57) ABSTRACT

Set forth are vascular valves and servovalves and methods of using the same that may be positioned in the circulatory system without interrupting blood flow to facilitate solid organ transplantation so as to reduce graft organ anoxia and degradation before and after harvesting and storage, thereby minimizing late-term cardiac allograft vasculopathy as a major cause of graft failure. Along the urinary tract, bypassing bladder-generated urge sensation by diverting urine directly from the ureters to a neobladder or collection bag alleviates intractable incontinence, nocturia, urethral syndrome, overactive bladder, dysuria, or frequency. Where the lower tract is missing, a prosthesis can thwart disease and preclude the degenerative metaplastic transition to carcinoma risked when gut is used to construct a conduit for urine, a neobladder, and/or high-maintenance stoma. Druglines to valves and servovalves can directly pipeline-target medication to nidi, anastomoses, or any other trouble spots.

### CROSS-REFERENCE TO RELATED APPLICATIONS

This continuation-in-part application follows provisional application Ser. No. 69/922,526, entitled Intravascular Valves and Servovalves—and Prosthetic Disorder Response Systems, filed on 13 Aug. 2019, which preceded parent nonprovisional application Ser. No. 16/873,914, filed on 11 Aug. 2020 retitled to Vascular Valves and Servovalves—and Prosthetic Disorder Response Systems, these earlier iterations herewith superseded and abandoned, in describing ductus side-entry jackets adapted to function as negative feedback-controlled continuously variable flow-diverting vascular servovalves, or highly damped nonsparking plunger solenoid-, and prospectively, Hasel or Peano-Hasel actuator-driven blood flow-diverting valves to impart fundamental and unprecedented improvements in solid organ transplantation and ischemia and hypoperfusion-free carotid endarterectomy, for example, among the capabilities vascular valves make possible.

Information pertaining to related devices such as stent-jackets and impasse-jackets are described in U.S. Pat. No. 11,389,171, entitled Integrated System for the Infixion and Retrieval of Implants; with ductus side-entry jackets described in U.S. Pat. No. 11,759,186, entitled Ductus Side-entry Jackets and Prosthetic Disorder Response Systems; and nonjacketing side-entry connectors, described in U.S. Pat. No. 11,013,858, entitled Nonjacketing Side-entry Connectors and Prosthetic Disorder Response Systems. Information pertaining to side-entry flow diversion jackets, vascular valves, servovalves, vascular servochokes, and nonjacketing side-entry connectors is incorporated by reference in its entirety.

Ductus side-entry jackets, vascular valves, servovalves, servochokes, and bidirectional side-entry diversion servovalves represent end-effectors in a fully implanted prosthetic disorder response system. Such a system can implement these devices to regulate the flow of blood or drugs in response to instructions encoded by a prescription programmer to control each in a set of multiple axes of a fully implanted hierarchical control system to treat multimorbid disease in a coordinated manner. Information pertaining to prosthetic disorder response systems per se will be found in copending application Ser. No. 17/689,880, entitled Prosthetic Disorder Response Systems.

This information provided here is intended to allow the presentation of multiple related inventions in an interrelated manner pursuant to which much of the inventive content disclosed here is to be submitted in divisional applications. These include but are not limited to compound vascular bypass solid organ transplantation, inline coupling jackets and tie-line type vascular prostheses and the automated placement thereof, confluence conduits; expandably structured lines and confluence conduits to accommodate growth in pediatric patients eliminating the need for later reentry; focused drip brachyradiochemotherapy, surgical chest domes to allow the avoidance of both mechanical ventilation and surgical pneumothorax; servochokes, implant heart protective cages, endothelial drumming and nanoparticle traps, transplantation-unassociated anastomosis bypasses; and one-, two-, or three-way passive side-entry flow diversion jackets.

Additional inventive content includes bidirectional side-entry diversion servovalves, negative feedback-controlled tuned shunts which adjust flow-through volume in response to sensors extended to monitor etiological foci and the cites of sequelary symptoms, ischemia-free carotid endarterectomy bypass and prostheses; tiny vessel grouped or gathered inlet/outlet expandable confluence conduits to reduce the duration of a compound vascular heart transplant in the smallest pediatric patients, donor to recipient-spanning bloodline, drugline, and confluence conduit switches to exchange a long for a short bloodline or the reverse with no interruption in blood flow, bidirectional side-entry servovalves, multiple input opening body surface ports cutaneous and subcutaneous, and lower urinary tract assist devices and prostheses.

PRIOR DISCLOSURE: Provisional application 69/922,526 filed on 13 Aug. 2019 and nonprovisional application Ser. No. 16/873,914, filed on 11 Aug. 2020.

### SECTION 1. FIELD OF THE INVENTION

The apparatus and methods to be described are intended for use by pediatric and adult cardiac, vascular, gastrointestinal, and thoracic surgeons, interventional cardiologists, radiologists, neuroradiologists and general, neurological, and veterinary surgeons to make possible continuously variable control over the volume of blood diverted into shunts or bypasses. Used thus, vascular servovalves make possible a fundamentally improved method for organ transplantation through the seamless, ischemia-free, continuous transfer from the circulatory system of the donor into that of the recipient by reciprocal cross-circulation, and seamless, or ischemia-free continuous vascular repairs by replacement of a native segment along a vessel with a prosthesis or a carotid endarterectomy, for example.

At the same time, use by urologists and endourologists makes possible the controllable diversion of urine from one or both ureters directly into a collection or drainage bag usually cinched about a thigh with outlet for emptying into a bathroom receptacle or into a disease- and defect-free lower part of the urinary tract, or into a prosthetic orthotopic ‘neobladder’ preferably made of synthetic materials or one surgically constructed using autologous or allogeneic, or if lined by urothelium, engineered tissue, wherewith either direct voiding into a bathroom receptacle or collection bag is made possible.

### SECTION 2. BACKGROUND OF THE INVENTION

2a. General

This continuation-in-part application describes ductus side-entry jackets modified as vascular valves or servovalves to divert urine from a ureter in order to bypass the lower urinary tract or an intervening portion thereof, or to controllably divert blood from a vessel through a catheter to an hypoxic organ or volume of tissue through a shunt or bypass, or to controllably divert blood to a transplant organ of a donor from the corresponding blood supply and drainage vessels of the recipient, thus seamlessly transferring the organ from the circulatory system of the donor into that of the recipient. Vascular servovalves can also be referred to as flow regulators or flow control valves. Tangentially, as the references make clear, use of the term ‘operation’ to denote a complex invasive procedure whereas the term ‘procedure’ should be reserved for a less complex or noninvasive procedure is routinely disregarded.

In consonance with a cardinal object of the invention, side-entry vascular valves and servovalves constitute the only means for gaining entry into the lumen of a blood vessel for the purpose of tapping off a proportion of the blood flowing through that lumen for redirection into another lumen without the need to transect the substrate vessel and thus introduce the risk of several serious complications. Other type valves can apportion flow between either of two outlets but must be positioned inline, necessitating transection of the substrate conduit.

While manually controllable by members of the operating team, vascular valves or servovalves are primarily intended to serve much as do electrostimulatory neuromodulation and cardiac desynchronization devices in the sense of serving as end-effectors responsive to the commands issued by the controller in a fully implanted automatic disorder response system responsive to sensor negative feedback in accordance with its prescription-program. As such, the scope of vascular valve and servovalve functionality extends to all serious chronic disorders addressed by internal medicine. In a monomorbid application, the receipt of sensor inputs and the release of medication in response thereto is under the control of a microcontroller chip.

Vascular valves are shown in FIGS. 2, 5, 7, and 8, a vascular servovalve in FIG. 10A, and the orientation of the flow diversion chute 18 in relation to the direction of flow in FIG. 15. Extension of the flow diversion chute into a passing column of blood diverts the fraction of the column that flows into the diversion chute behind its front tip upturned in the direction opposite to that of the column into a synthetic or catheteric line, or bloodline. By controlling the degree of this extension into the column, the vascular valve controls the volume of blood diverted into the bloodline at its inlet end.

As shown in FIG. 15, in a compound vascular bypass solid organ transplant, another valve at the opposite or outlet end of the bloodline functions in synchrony with but opposite to that at the inlet end. In the drawing figures, part number 196 denotes a bloodline whether synthetic, autograft, allograft, or xenograft, conveying the arterial, oxygenated, flow through a single vessel, part number 230 denotes a confluence conduit which conveys the combined flow of plural vessels carrying oxygenated blood whether tubular, anatomical, or as shown in FIGS. 15C, 15D, and 36F, flats 233 and vaults 234 expandable to accommodate growth in a neonate, while part number 198 denotes a bloodline or tie-line regardless of type.

In the figures showing vascular valves 2, 5, 7, 8 and a servovalve 10A, the substrate vessel lumen is 1, its wall 2, endothelial or urothelial lining of the operator-proximal luminal wall, 13, the urothelial lining of the operator-distal luminal wall, 14, and 15 are suture loops to pass through suture if the vessel with jacket should be drawn away from encroaching upon neighboring tissue. Part numbers 8 and 8′ are druglines that make possible the direct release of medication or maintenance agents into these devices and the blood, urine, or any other fluid produced by the body flowing through them. Vascular valves do not control the delivery of drugs through druglines; when not gravity fed, flow out of small drug reservoirs implanted subcutaneously in the pectoral region is by means of an inline axial or peristaltic pump.

In FIG. 30, two pairs of druglines are provided, one toward the top of the FIGS. 8 and 8′ and another leading upward from the body surface port at the bottom of the figure also 8 and 8′. Entry through the latter into their respective valves is through a spring-loaded one-way cap (not shown) that prevents the leakage of urine in the caudal direction and allows the craniad passing through of a probe to remove a test sample or inject drugs. Using the lower druglines, medication of a maintenance agent such as a crystal solvent can also be injected at the body surface port.

In the treatment of comorbid or multimorbid disease, a master control microprocessor is programmed, usually in Forth, to administer a hierarchical control system such as addressed below in section 2b(1)(a), entitled Sudden Compound Vascular Bypass, or Sudden Switched-organ, Transplantation, in the subsection thereof entitled Implanted Simple and Hierarchical Control Systems, in which each morbidity or organ system, for example, is assigned an arm of control at the sensor ground level. This feedback is then fed up and coordinated at an intermediate level and from there to the highest level of control to achieve the optimal state of homeostasis for the specific patient. Code prepared for this purpose is not entrusted to artificial intelligence which for now lacks the competency essential for medical application.

The maintenance of proper flow through side-entry jackets, vascular valves, nonjacketing side-entry connectors and servovalves is addressed below in the subsection entitled Avoidance of Disruptions in Reciprocal Cross-circulation to Include Valve-reactive. To assure their patency, side-entry valve bloodlines and druglines can be provided with an intermittent drop of a heparin and the substrate vessel with a nitrate, usually post-liver metabolism-modified nitroglycerin (see, for example, Kim; K. H., Kerndt; C. C., Derek, G. A., and Schaller, D. J. 2023. “Nitroglycerin,” Online, Treasure Island, Florida: StatPearls Publishing Co.). Nonjacketing side-entry connector secured delivery can be as depicted in FIG. 11 of U.S. Pat. No. 11,013,858, entitled Nonjacketing Side-entry Connectors and Prosthetic Disorder Response Systems, Patients with blood dysstasias such as sickle cell disease, thalassemia, or hemophilia must be treated to ameliorate this complication before initiating an organ transplant.

With respect to FIGS. 28 thru 30, the kidneys upstream to the urine tap-off valves, stone formers should be treated with a systemic stone suppression medication (see, for example, Institute for Quality and Efficiency in Health Care (IQWIG) 2023. “Kidney Stones: Learn More—Treatment Options for Kidney Stones, Online, Cologne, Germany: InformedHealth.org [Internet], available at https://www.ncbi.nlm.nih.gov/books/NBK348939/; Xiao, Y, and Xiao, Z. 2023. “Association between Serum Klotho and Kidney Stones in US Middle-aged and Older Individuals with Diabetes Mellitus: Results from 2007 to 2016 National Health and Nutrition Survey,” Online, American Journal of Nephrology (Basel, Switzerland) 54(5-6): 224-233; Finkielstein, V. A. and Goldfarb, D. S. 2006. “Strategies for Preventing Calcium Oxalate Stones,” Online, Canadian Medical Association Journal (Ottawa, Canada) 174(10): 1407-1409).

A crystal solvent can also be directly pipe-targeted into the valves and substrate ureters as programmed. For the reciprocal cross-circulation pattern used to connect the corresponding vessels of the donor and recipient in a compound vascular bypass orthotopic heart transplant as depicted in FIGS. 15 and 16 to proceed without interruption, it is essential that flow through each of the bloodlines pass freely.

Any indication of an interruption in flow such as due to clotting is immediately detected by thin film blood pressure or pulse sensors lining the valve outflow channel through sidestem 19 in FIGS. 2, 5, 7, 8, and 10A which signal the disorder response system master control microprocessor to release an anticoagulant such as apixaban or a heparin and/or a positive inotrope into the line through druglines 8 and/or 8′ of every vascular servovalve. The Xa inhibitor with the quickest onset of action currently available is edoxaban (McCarty, D. and Robinson, A. 2016. “Factor Xa Inhibitors: A Novel Therapeutic Class for the Treatment of Nonvalvular Atrial Fibrillation,” Online, Therapeutic Advances in Cardiovascular Disease (London, England) 10(1): 37-49).

When numerous bloodlines are needed to connect numerous arteries and veins as in a heart transplant, to save time and simplify the surgical field, those calling for the same treatment can be combined though a confluence conduit, these described in section 2b(1)(b)(b14), entitled Confluence Conduits: Connection of One or More Bloodlines to Flow through a Common Channel to the Same or a Different Number of Bloodlines with Self-leveling Apportionment of Outflow, which incorporates at least one drugline and will usually be large enough in internal diameter to allow the positioning of a miniature inline axial pump to propel or retard the rate of flow-through if necessary.

In the drawing figures, arterial bloodlines, or tie-lines, and tie-line type prostheses are assigned part number 196 and those venous 196′. Whether tubular or anatomically shaped, the former convey the blood flow through a single vessel, whereas a confluence conduit conveys the flow of more than one vessel and when arterial are assigned part number 230 and when venous part number 230′. In FIGS. 15A thru 15D, the cardiac and supraaortic arteries are multiple calling for the application of confluence conduits. Venous return to heart through the large superior and inferior venae cavae, two bloodlines 196 are used. In FIGS. 15A thru 15D, the schematic representation shows three arterial vessels which might or might not be those to which the drawings suggest are connected to the heart vessels indicated.

This directly pipe-targeted release of medication into the valve and its substrate bloodline eliminates the need to disperse the drug throughout the systemic circulation where it comes into contact with tissue in which it induces an adverse reaction, so that the drug can be used in a higher concentration without the side effects otherwise associated with the drug. Once fixed in position, vascular valves and servovalves are almost always controlled by a fully implanted disorder response system described in U.S. Pat. No. 11,013,858, entitled Nonjacketing Side-entry Connectors and Prosthetic Disorder Response Systems, U.S. Pat. No. 11,759,186, entitled Ductus Side-entry Jackets and Prosthetic Disorder Response Systems, and copending application Ser. No. 17/689,880, entitled Prosthetic Disorder Response Systems, as well as in the present document.

With regard to immunosuppressant, oncological medication, and many others drugs intended for a target organ or volume of tissue, because it directly pipe-targets and substantially confines drugs to the intended target, a fully implanted automatic disorder response system minimizes if not eliminates the adverse reactions of systemic dosing to which all the tissues of the body are exposed. At the same time, the system can release a background dose of medication such as a chemotherapeutic to destroy suspect occult malignant “daughter” cells shed into the systemic circulation from a known malignant tumor in a lower dose than if the tumor were included in the dose.

In the directly pipe-targeted delivery of immunosuppressants by a fully implanted automatic disorder response system to one or more allografts as well as the native thymus and spleen, the benefits provided by such a system consist not only in the continued, usually daily, timeliness of release and the avoidance of nontargeted tissue but quite likely in allowing for a concurrent background systemic dose to target the bone marrow, lymph nodes, and vascular tree in a lesser sum systemic dose.

Generally, the implantation of multiple organs from the same or different donors calls for an increase in the sum dose of immunosuppressants relative to what would be the dose for any one of the organs where due to the combined chimerizing effect of the set, a lesser dose is needed for each organ. The addition of one or more allografts, especially a lung, as would necessitate increasing the sum dose would then necessitate increasing the dose, but still to a lesser dose to avoid further rendering the already impaired patient susceptible to infection and cancer. Multiple organ transplantation is further addressed below in section 2a(2), entitled Inventive Content-pertinent Fetal Diagnostics.

Broadly, while the transplantation of multiple graft organs taken from the same donor can at the same time exert a counteracting immune tolerance inducing effect, immunological response to the implantation of allogeneic tissue is proportional in magnitude to the magnitude of the insult, increasing with multiple transplant organs and/or multiple donors, which implicit in the literature, must be inferred (see, for example, Gruessner, R. W. G. and Gruessner, A. C. 2023. “Dual Living Donor Organ Transplants,” Clinical Transplantation (Copenhagen, Denmark) 37(4): e14923; Li, Q. and Lan, P. 2023. “Activation of Immune Signals during Organ Transplantation,” Online, Signal Transduction and Targeted Therapy (London, England) 8(1): 110; Wang. L., Zhang, Y., Yu, H., Song, J., and Wang, Y. 2023. “Sequential Transplantation of the Liver-Kidney-Heart from Different Donors: A Case Report,” Online, European Heart Journal. Case Reports (Oxford, England) 7(10): ytad472; Hsiao, S. and Khush, K. K. 2022. “Donor Selection for Multiorgan Transplantation,” Current Opinion in Organ Transplantation (Hagerstown, Maryland) 27(1): 52- 56; Kovac, D., Choe, J., Liu, E., Scheffert, J., Hedvat, J., and 4 others 2021. “Immunosuppression Considerations in Simultaneous Organ Transplant,” Pharmacotherapy (Malden, Massachusetts) 41(1): 59- 76; Magruder, J. T., Suzuki, Y., Sperry, A., Vasquez, C. R., Smood, B., and 7 others 2020. “Multiorgan Procurement is Associated with a Survival Benefit after Heart Transplantation,” Clinical Transplantation (Copenhagen, Denmark) 34(8): e13901; Sieńko, J., Kotowski, M., Paczkowska, E., Sobuś, A., Tejchman, K., and 4 others 2018. “Correlation between Stem and Progenitor Cells Number and Immune Response in Patients after Allogeneic Kidney Transplant,” Online, Annals of Transplantation (Smithtown, New York) 23:874-878; Todd, J. L. and Palmer, S. M. 2017. “Danger Signals in Regulating the Immune Response to Solid Organ Transplantation,” Online, Journal of Clinical Investigation (Ann Arbor, Michigan) 127(7): 2464-2472; Georgel, P. 2016. “Innate Immune Receptors in Solid Organ Transplantation,” Human Immunology (New York, New York) 77(11): 1071-1075; Cano. R. L. E. and Lopera, H. D. E. 2013. “Introduction to T and B Lymphocytes,” in Anaya, J. M. Shoenfeld, Y., Rojas-Villarraga, A., Levy, R. A., and Cervera R. (eds), Autoimmunity: From Bench to Bedside, Online, Bogota, Colombia: El Rosario University Press; Loebe. M. 2011. “Multiple-organ Transplantation from a Single Donor,” Online, Texas Heart Institute Journal (Houston, Texas) 38(5): 555-558; Chaplin, D. D. 2010. “Overview of the Immune Response,” Online, Journal of Allergy and Clinical Immunology ( )St. Louis, Missouri) 125(2 Supplement 2): S3-23; Pinderski, L. J., Kirklin, J. K., MeGiffin, D., Brown, R., Naftel, D. C., and 11 others 2005. “Multi-organ Transplantation: Is There a Protective Effect against Acute and Chronic Rejection?,” Journal of Heart and Lung Transplantation (New York, New York) 24(11): 1828-1833.

Replete with variables and necessarily tailored to the individual patient, specific information as to the adjustment in the administration of immunosuppressants for a heart transplant recipient who later undergoes a kidney, liver, or lung transplant or the difference dosing in a combined heart and kidney as opposed to a combined heart, lung, and kidney transplant, for example, is not squarely addressed (see, for example, Szumilas, K., Wilk. A., Wiśniewski, P., Gimpel. A., Dziedziejko. V., Kipp, M., and Pawlik, A. 2023. “Current Status Regarding Immunosuppressive Treatment in Patients after Renal Transplantation,” Online, MDPI [Multidisciplinary Digital Publishing Institute] International Journal of Molecular Sciences (Basel, Switzerland) 24(12): 10301; Mellon, L., Doyle, F., Hickey, A., Ward, K. D., de Freitas, D. G., and 3 others 2022. “Interventions for Increasing Immunosuppressant Medication Adherence in Solid Organ Transplant Recipients,” Online, Cochrane Database of Systematic Reviews (Chichester, West Sussex, England) 9(9): CD012854; Anger, L. B. and Doligalski, C. T. 2021. “Solid Organ Transplant Pharmacotherapy: Complicated and Continually Changing,” Pharmacotherapy (Malden, Massachusetts) 41(1): 4-5; Ferede, A. A., O'Connell, C., Davis, N. F., Mohan, P., Robertson, I., O'Kelly, P., and Little, D. M. 2021. “The Irish Experience of Kidney Transplantation among Recipients with Prior Non-renal Solid Organ Transplants: A Retrospective Study on Short- and Long-term Outcomes,” Clinical Transplantation (Copenhagen, Denmark) 35(1): e14156; Kovac. D., Choe. J., Liu, E., Scheffert. J., Hedvat, J., and 4 others 2021, Op cit., preceding paragraph; Monchaud, C. Marquet, P. 2009. “Pharmacokinetic Optimization of Immunosuppressive Therapy in Thoracic Transplantation: Part I,” Online, Clinical Pharmacokinetics 48(7): 419-462; Duncan, M. D. and Wilkes, D. S. 2005. “Transplant-related Immunosuppression: A Review of Immunosuppression and Pulmonary Infections,” Online, Proceedings of the American Thoracic Society (New York, New York) 2(5): 449-455).

Provided the implanted rejection sensors as specified below in this section indicate that it is justified, the system can also reduce this dosing over time elapsed since the transplant “As the time after transplant increases, physicians generally decrease the dose of immunosuppressive agents on an empirical basis, testing small changes to see whether side effects can be reduced without threatening graft rejection” Field, M. J. 2000. “Immunosuppressive Drugs for Transplant Patients,” Chapter 5 in Lawrence, R. L. and Zwanziger, L. (eds.), Extending Medicare Coverage for Preventive and Other Services, Washington, DC: National Academies Press. Inherently providing personalized data, the implanted sensors eliminate much of this conventional trial and error or hit and miss method for ascertaining optimal dosing.

The risk of cancer, especially lung cancer, following a heart transplant also calls for an increase in the dosing of immunosuppressants (see, for example, Lateef, N., Farooq, M. Z., Latif, A., Ahmad, S., Ahsan, M. J., and 8 others 2022. “Prevalence of Post-heart Transplant Malignancies: A Systematic Review and Meta-Analysis,” Current Problems in Cardiology (Amsterdam, North Holland, Netherlands) 47(12): 101363; Youn, J. C., Kim D. Kim I C, Lee H S, Choi J O, and 9 others 2022. “Characteristics, Outcomes, and Predictors of de novo Malignancy after Heart Transplantation,” Online, Frontiers in Cardiovascular Medicine (Lausanne, Switzerland) 9:939275; Heil, K. M., Helmschrott, M., Darche, F. F., Bruckner, T., Ehlermann, P., and 6 others 2021. “Risk Factors, Treatment, and Prognosis of Patients with Lung Cancer after Heart Transplantation,” Online, MDPI [Multidisciplinary Digital Publishing Institute] Life (Basel, Switzerland) 11(12): 134; Youn, J. C., Stehlik, J., Wilk. A. R., Cherikh, W., Kim, I. C., and 10 others 2018. “Temporal Trends of de Novo Malignancy Development after Heart Transplantation, Online, Journal of the American College of Cardiology (New York, New York) 71(1): 40-49; Rivinius, R., Helmschrott, M., Ruhparwar, A., Schmack, B., Klein, B., and 10 others 2014. “Analysis of Malignancies in Patients after Heart Transplantation with Subsequent Immunosuppressive Therapy,” Online, Drug Design, Development, and Therapy (Auckland, New Zealand) 17:9:93-102).

In prospective late prenatal, neonatal, infant, and toddler compound bypass heart transplantation, grouped tiny vessel device inlet entered and outlet exited growth-expandable confluence conduits are shown in FIGS. 15C and 15D and addressed below in section 2b(1)(b)(b14), entitled Confluence Conduits: Connection of One or More Bloodlines to Flow through a Common Channel to the Same or a Different Number of Bloodlines with Self-leveling Apportionment of Outflow, in the subsection thereof entitled Tiny Vessel Grouped Inlet/Outlet Expandable Confluence Conduits, and in section 6, entitled Description of the Preferred Embodiments of the Invention, in the subsection thereof entitled Tiny Vessel Grouping Device.

Tiny vessel grouping is an interim expedient devised to avert the need to apply a separate valve to each of the small arteries in a compound vascular bypass heart transplant on a neonate, or prospectively, a prenate. After an interval during which the baby is allowed to grow and gain in strength, the grouping devices can be removed without an interruption in blood flow by bypassing each vessel as depicted in FIG. 15D. When a larger autologous or autograft vessel segment is available, to anastomose the grouped small vessels to a single bypass is also an option but does not gain time. However, use of a humanized xenograft, tissue engineered autograft, or an allograft taken from a deceased prenate or neonate having been sustained in a deceased donor sustainment center as described in this section in the subsection below entitled Deceased Donor Sustainment Centers of this section avoids a preliminary procedure to harvest a graft vessel carrying the usual risks pertaining to any surgical procedure as well as the risk of a loss in function related to the graft taken.

Another means for achieving size and weight reduction to minimize if not eliminate obtrusiveness as is imperative in a neonate is the use of bidirectional valves as addressed below in section 2b(2)(b), entitled Bidirectional Valves for Perinatal Extracardiac Correction of Complete or Dextro-transposition of the Great Arteries and Reciprocal Cross-circulation, illustrated in drawing FIGS. 12 thru 13B where limitation to the application cited should not be implied. Based upon sensor feedback to the automatic disorder control system master control microprocessor, druglines 8 allow the direct release into side-entry devices and substrate lumen of medication and/or maintenance solutions when necessary to dispel the formation of clot or a biofilm, for example. Acclimation. and depending upon the anatomical context, fibrous encapsulation should soon dispel any discomfort.

This is expedited by connecting the bypasses with a simple side-entry diversion jacket at either end, to be preferred for their druglines, part numbers 8 and 8′ in the drawing figures, which allow the direct pipe-targeting of immunosuppressants, steroids, and/or antimicrobials when an adverse reaction to include rejection is anticipated. Simple side-entry diversion jackets proportional in size to the substrate ductus and light in weight, conceptualizing this as loading the tiny vessels with obtrusive hardware is misconceived. In FIGS. 15A, 15C, and 15D, the appearance of connection to the supraaortic arteries is meant as exemplary, any other three or more arteries no less applicable.

In FIGS. 15C and 15D, grouped tiny vessel device entry into and exit from growth-expandable confluence conduits allow multiple vessels to pass common bistable linear plunger solenoid- or if determined preferable, servomotor-driven, blood flow diversion chutes to divide the flow of blood between one fraction that continues through the substrate vessel and a second fraction that is diverted into the confluence conduit at the entry or donor end and the exit or recipient end. The handling of cardiac vessels prone to vasospasm and the induction of arrhythmias, precise synchrony is obtained by actuating the valves simultaneously from the same switch, and having means for reestablishing synchronization immediately at hand. In FIG. 15C, the orientation of the grouping device at each corner of the figure is in the direction of flow.

To sites susceptible to clot, the automatic implanted disorder response system institutes an intermittent pipe-targeted drip of a heparin to the side-entry valve, for example, thus avoiding the potentially serious complications encountered when a heparin is systemically dispersed. Specifically, dispersed throughout the circulation, heparin can induce a dangerous dose-dependent thrombocytopenia, osteopenia, anaphylaxis with its multiple adverse symptoms, and problem bleeding such as susceptibility to the formation of a subdural hematoma following an injury to the head, and numerous less serious complications. At the same time, the substrate vessel can be provided with an intermittent drop of a nitrate, usually nitoglycerin (Nicolas, D., Nicolas, S., Hodgens; A., and Reed, M. 2023. “Heparin-Induced Thrombocytopenia,” Online, Treasure Island, Florida: StatPearls Publishing Co.; National Institute of Diabetes and Digestive and Kidney Diseases 2012. LiverTox: Clinical and Research Information on Drug-induced Liver Injury [Internet]. Bethesda Maryland Heparin. Available from: https://www.nebi.nlm.nih.gov/books/NBK548126; Krishnamurthy, M. and Freedman, M. L. 2005. “Complications of Anticoagulation with Heparin. Online, [American Medical Association] Virtual Mentor (Chicago, Illinois) 7(4): virtualmentor.2005.7.4.cpr11-0504; MacLean, J. A., Moscicki, R., and Bloch, K. J. 1990. “Adverse Reactions to Heparin,” Annals of Allergy (McLean, Virginia) 65(4): 254-259). At the same time, the substrate vessel can be provided with an intermittent drop of a nitrate, usually nitroglycerin.

The U.S. Patents specified above under the opening section entitled Cross Reference to Related Applications describe at least three methods for preventing the continued flow of heparin or any other superparamagnetically nanoparticle carrier-bound agent past the target and into the systemic or pulmonary circulation. Accordingly, the incorporation of at least one accessory channel into which a drugline can be inserted for the system controller to directly target medication to any or every vascular valve, servovalve, and into its substrate vessel is an indispensable factor to assure uninterrupted flow through that device and substrate bloodline and that any accumulation of a systemic or nontargeted dose of heparin will be far too slight to cause complications.

A relatively simple implanted microcontroller chip, drug reservoir, drugline, and side-entry jacket as addressed in U.S. Pat. No. 11,759,186, entitled Ductus Side-entry Jackets and Prosthetic Disorder Response Systems, are adequate to program the targeted release of an anesthetic or any other drug of pain-relieving substance directly into the blood supply of an organ or a volume of tissue. Any one such jacket can release medication that is precisely end-arterial blood supply tissue targeted or discharged into the systemic or pulmonary circulation.

A nonjacketing side-entry connector as addressed in U.S. Pat. No. 11,013,858, entitled Nonjacketing Side-entry Connectors and Prosthetic Disorder Response Systems allows the same action where the target is an organ or a volume of tissue. The ability to precisely target pain killing medicinals such as lidocaine with adrenaline when necessary to extend the duration of anesthesia should allow a subsidence in the underuse of opioids, for example, to dispel the concerns of practitioners for the possibility of legal repercussions. A complementary process is provided by intrathecal drug delivery systems (see Ontario Health 2024 below).

Released at a consistent interval, this can alleviate intense pain on a continuous basis that would otherwise require administration of a local anesthetic in a clinic and then to remain effective for only a short while (see, for example, Hu. Y.-D., Wang, Z.-D., Yue, Y.-F., Li, D. Zhen. S.-Q., and 5 others 2024. “Inhibition of HDAC6 [histone deacetylase 6] Alleviates Cancer-induced Bone Pain by Reducing the Activation of NLRP3 [NOD [nucleotide-binding oligomerization domain]-like Receptor Pyrin Domain Containing 3 Inflammasome Activity] Inflammasome,” Online, International Journal of Molecular Medicine (Athens, Greece) 53(1): 4; Ontario Health 2024. “Intrathecal Drug Delivery Systems for Cancer Pain: A Health Technology Assessment,” Ontario Health Technology Assessment Series (Toronto, Ontario, Canada) 24(2): 1-162; Mestdagh, F., Steyaert, A., and Lavand'homme, P. 2023. “Cancer Pain Management: A Narrative Review of Current Concepts, Strategies, and Techniques,” Online, MDPI [Multidisciplinary Digital Publishing Institute] Current Oncology (Basel, Switzerland) 30(7): 6838-6858; Queremel Milani; D. A. and Davis, D. D. 2023. “Pain Management Medications,” Online, Treasure Island, Florida: StatPearls Publishing Co.; Ruggiero, E., Pambuku, A., Caccese, M., Lombardi, G., Gallio, I., and 3 others 2023. “Case Report: The Lesson from Opioid Withdrawal Symptoms Mimicking Paraganglioma Recurrence during Opioid Deprescribing in Cancer Pain,” Online, Frontiers in Pain Research (Lausanne, Switzerland) 4:1256809; Yang H, Wang Y, Zhen S, Wang B, Jiao M, and 4 others 2023. “AMPK [adenosine monophosphate-activated protein kinase] Activation Attenuates Cancer-induced Bone Pain by Reducing Mitochondrial Dysfunction-mediated Neuroinflammation,” Online, Acta Biochimica et Biophysica Acta Sinica (Shanghai, China) 55(3): 460-471; Brozovic, G., Lesar, N., Janev, D., Bošnjak, T., and Muhaxhiri, B. 2022. “Cancer Pain and Therapy,” Online, Acta Clinica Croatica (Zagreb, Croatia) 61 (Supplement 2): 103-108; Zhang C, Huang Y, Ouyang F, Su M, Li W, and 4 others 2022. “Extracellular Vesicles Derived from Mesenchymal Stem Cells Alleviate Neuroinflammation and Mechanical Allodynia in Interstitial Cystitis Rats by Inhibiting NLRP3 [NOD-like receptor pyrin domain containing 3 inflammasome activity] Inflammasome Activation,” Online, BioMed Central Journal of Neuroinflammation (London, England) 19(1): 80; Chong, P. H. and Yeo, Z. Z. 2021. “Parenteral Lidocaine for Complex Cancer Pain in the Home or Inpatient Hospice Setting: A Review and Synthesis of the Evidence,” Online, Journal of Palliative Medicine (Larchmont, New York) 24(8): 1154-1160; Manirakiza, A., Irakoze, L., Manirakiza, S., and Bizimana, P. 2020. “Efficacy and Safety of Fentanyl Compared with Morphine among Adult Patients with Cancer: A Meta-analysis, Online, East African Health Research Journal (Bujumbura, Burundi) 4(1): 8-16; Wolff, R. F., Aune, D., Truyers, C., Hernandez, A. V., Misso, K., Riemsma, R., and Kleijnen, J. 2012. “Systematic Review of Efficacy and Safety of Buprenorphine versus Fentanyl or Morphine in Patients with Chronic Moderate to Severe Pain,” Current Medical Research and Opinion (Newbury, Berkshire, England) 28(5): 833-845).

Optically based oximetric sensors in development may be adaptable for such use in the future (see, for example, Lee, H. S., Noh, B., Kong, S. U., Hwang, Y. H., Cho, H. E., Jeon, Y., and Choi, K. C. 2023. “Fiber-based Quantum-dot Pulse Oximetry for Wearable Health Monitoring with High Wavelength Selectivity and Photoplethysmogram Sensitivity,” Online, Nature Partner Journals Flexible Electronics (Basingstoke, England) 7(1): 15). Hosanee, M., Chan, G., Welykholowa, K., Cooper, R., Kyriacou, P. A., and 11 others 2020. “Cuffless Single-site Photoplethysmography for Blood Pressure Monitoring,” Online, MDPI [Multidisciplinary Digital Publishing Institute] Journal of Clinical Medicine (Basel, Switzerland) 9(3): 723; Nitzan, M., Nitzan, I., and Arieli, Y. 2020. “The Various Oximetric Techniques Used for the Evaluation of Blood Oxygenation, Online, MDPI [Multidisciplinary Digital Publishing Institute] Sensors (Basel, Switzerland) 20(17): 4844).

The subsection entitled Definition of Shunts, Passive and ‘Tuned,’ or Metered, and Bypasses, Passive and Metered just preceding section 2b(2)(c) below, entitled Vascular Segment Prostheses: Inline Coupling Jackets and Bridging, or Tie-lines, explains the distinctions among these devices. In compound vascular bypass organ transplantation, the meaning of bypass is distinct in pertaining to bloodlines that connect corresponding vessels of a recipient to those of a donor. In the context of the invention, when connected to the same side-entry device, a drug line is a drugline or sideline, and a blood line is a bloodline or mainline.

### Overview

A central object of the methods and apparatus to be described is to make possible surgical operations such as a solid organ transplantation and a carotid endarterectomy without the need to interrupt the flow of blood. This capability and the institution of preoperative measures to completely exclude ischemia, cardiopulmonary bypass, cardioplegia, hypothermia, and general anesthesia, plus the follow-up use of a fully implanted disorder response system to monitor and immediately respond to any subsequent adverse sequela to the operation reduces if not eliminates causes for postoperative complications and may well continue do so for the life of the patient.

The possible adverse sequelae such as ischemia-reperfusion injury associated with a conventionally executed such operation can not only contribute to the short- or long-term rejection of an allograft heart, for example, and the exclusively systemic administration of immunosuppressants in a higher dose risk infection and cancer. Any surgical operation that necessitates the use of cardiopulmonary bypass or clamping subjects the affected organs or tissues to ischemia. The adverse sequelae attributable to ischemia during heart and great vessel surgery are documented in the sections to follow. Side-entry devices are intended to eliminate this problem.

The means for the repair of complex congenital anomalies and injury to the great and other major vessels presented below in section 2b(2)(c), entitled Vascular Segment Prostheses: Inline Coupling Jackets and Bridging, or Tie-lines, and the sections subordinate to it can and should supplant all forms of hypothermic circulatory arrest. More specifically, the means laid out do not require an interruption in blood flow through the subject vessel such as the aortic arch or carotid artery and therefore eliminate the potential for injury to the brain. Furthermore, as addressed in this and other sections, conventional repairs of these and other major vessels pose considerable risks, tend to lack long-term durability, and for placement in a child, lack the ability to expand in step with growth, necessitating painful reentries. The expandable structure addressed below in section 2b(2)(f), entitled Expandable Structure to Accommodate Growth and Self-adjust in Size in Vascular Segment Prostheses, Lines, and Confluence Conduits Placed in Children, seeks to reduce if not eliminate a need for later reentry.

For applicable operations such as intracranial and aortic arch repairs lasting less than an hour, the use of deep hypothermic circulatory arrest to protect the brain still has utility (see, for example, Kulyabin, Y. Y, Bogachev-Prokophiev, A. V., Soynov, I. A., Omelchenko, A. Y., Zubritskiy, A. V., and Gorbatykh, Y. N. 2020. “Clinical Assessment of Perfusion Techniques during Surgical Repair of Coarctation of Aorta with Aortic Arch Hypoplasia in Neonates: A Pilot Prospective Randomized Study,” Seminars in Thoracic and Cardiovascular Surgery (Philadelphia, Pennsylvania) 32(4): 860-871; Dorotta, I., Kimball-Jones, P., and Applegate, R. 2nd 2007. “Deep Hypothermia and Circulatory Arrest in Adults, Seminars in Cardiothoracic and Vascular Anesthesia (Thousand Oaks, California) 11(1): 66-76).

However, not all complex operations can be completed in one hour. The concept is perpetuated in the chilling function incorporated into cardiopulmonary bypass machines, which allows an interruption in the flow of blood to the brain longer than one hour. More recent versions of hypothermic circulatory arrest reduce the degree of hypothermia from deep to moderate and combine this with an option as to the direction of perfusion, usually antegrade, which neither significantly increases the usable duration nor eliminates the risk of adverse cognitive sequelae.

The complications of hypothermia include coma, pancreatitis, frostbite resulting in gangrene, ataxia, acute kidney injury, hyperkalemia, pulmonary edema, atrial and ventricular arrhythmias, pulseless electrical activity, and death (see, for example, Hieu Duong, H and Patel, G. 2024. “Hypothermia,” Online, Treasure Island, Florida: StatPearls Publishing Col; Tanaka, A., Chehadi, M., Smith, H. N., Hassan, M., Sandhu, H. K., and 3 others 2023. “Deep Hypothermic Circulatory Arrest with Retrograde Cerebral Perfusion: How Long Is Safe?,” Annals of Thoracic Surgery (Amsterdam, North Holland, Netherlands) 116(1): 27-33; Guo, S., Sun, Y., Ji, B., Liu, J., Wang, G., and Zheng, Z. 2015. “Similar Cerebral Protective Effectiveness of Antegrade and Retrograde Cerebral Perfusion during Deep Hypothermic Circulatory Arrest in Aortic Surgery: A Meta-analysis of 7023 Patients,” Artificial Organs (Cambridge, Massachusetts) 39(4): 300-308; Oppido, G., Pace Napoleone, C., Turci, S., Davies, B., Frascaroli, G., and 3 others 2006. “Moderately Hypothermic Cardiopulmonary Bypass and Low-flow Antegrade Selective Cerebral Perfusion for Neonatal Aortic Arch Surgery,” Annals of Thoracic Surgery (Amsterdam, North Holland, Netherlands) 82(6): 2233-2239). Hypothermic arrest is also combined with cardiopulmonary bypass and extracorporeal membrane oxygenation. Broadly, circulatory arrest carries serious risks for causing neurological and other damage regardless how it is accomplished.

Used less often than cardiopulmonary bypass, the complications posed by hypothermic circulatory arrest in any of its forms and the general anesthesia it requires arise mostly during rewarming, which the sudden and metered compound vascular bypass methods and automated means for replacing a major vessel with a prosthesis—a prosthesis able to grow with a child—completely avoid. An extensive literature documents these complications (see, for example, Hughes, G C, Chen E P, Browndyke J N, Szeto W Y, DiMaio J M, and 9 others 2024. “Cognitive Effects of Body Temperature during Hypothermic Circulatory Arrest Trial (GOT ICE): A Randomized Clinical Trial Comparing Outcomes after Aortic Arch Surgery,” Circulation 149(9): 658-668; Montisci, A., Maj. G., Cavozza, C., Audo, A., Benussi, S., and 4 others 2023. “Cerebral Perfusion and Neuromonitoring during Complex Aortic Arch Surgery: A Narrative Review,” MDPI [Multidisciplinary Digital Publishing Institute] Journal of Clinical Medicine (Basel, Switzerland) 12(10): 3470; Miranda, D., Maine, R., Cook, M., Brakenridge, S., Moldawer, L., and 6 others 2021. “Chronic Critical Illness after Hypothermia in Trauma Patients,” Trauma Surgery and Acute Care Open (London, England) 6(1): e000747; Qu, J. Z., Kao, L. W., Smith, J. E., Kuo, A., Xue. A., and 3 others 2021. “Brain Protection in Aortic Arch Surgery: An Evolving Field, Journal of Cardiothoracic and Vascular Anesthesia ((Philadelphia, Pennsylvania) 35(4): 1176-1188; Okita, Y., Miyata, H., Motomura, N., and Takamoto, S. 2015. “A Study of Brain Protection during Total Arch Replacement Comparing Antegrade Cerebral Perfusion versus Hypothermic Circulatory Arrest, with or without Retrograde Cerebral Perfusion: Analysis Based on the Japan Adult Cardiovascular Surgery Database,” Journal of Thoracic and Cardiovascular Surgery (St. Louis, Missouri) 149(2 Supplement): S65-S73; Vaity, C., Al-Subaie, N., and Cecconi, M. 2015. “Cooling Techniques for Targeted Temperature Management Post-cardiac Arrest,” BioMed Central Critical Care (London, England) 19(1): 103; Zhang, Y. P., Zhu, Y. B., Duan, D. D., Fan, X. M., He. Y., Su, J. W., and Liu, Y. L. 2015. “Serum UCH-L1 [ubiquitin C-terminal hydrolase L1] as a Novel Biomarker to Predict Neuronal Apoptosis following Deep Hypothermic Circulatory Arrest,” International Journal of Medical Sciences (Sydney, Australia) 12(7): 576-582).

Here there will be laid out the application of side-entry and inline coupling devices to allow the supersession of operations that conventionally necessitate a significant interruption in the delivery of oxygen to the brain thereby eliminating the many complications both risk. These include cardiopulmonary bypass and deep hypothermic circulatory arrest. Solid organ transplantation to include that of the heart with the aid of side-entry vascular valves or servovalves is covered in sections 2a, entitled Background of the Invention. General, and 2b(1), entitled The Intercorporeal Diversion of Blood Flow, and its subordinate sections.

With respect to cardiovascular surgery, the two methods presented here provide fundamental advantages over conventional methods, a third applicable to any and all of medicine and surgery. Complex repairs of congenital malformities of the aortic arch and supraaortic branches, great and other major vessels are disposed of along with their long list of complications by resorting to the seamless ischemia-free replacement of the defective segment, with branches if applicable, through the use of an automatically placed prosthesis secured at either end by inline coupling jackets, which are neither valves nor entered through a side of the substrate ductus. Such prostheses and the inline coupling jacket used to emplace and secure them are covered in sections 2a, entitled Background of the Invention. General, and 2b(2)(c), entitled Vascular Segment Prostheses: Inline Coupling Jackets and Bridging, or Tie-lines.

Both include the use of an automatic implanted prosthetic disorder response system addressed in U.S. Pat. No. 11,759,186, entitled Ductus Side-entry Jackets and Prosthetic Disorder Response Systems, U.S. Pat. No. 11,013,858, entitled Nonjacketing Side-entry Connectors and Prosthetic Disorder Response Systems, and copending application Ser. No. 17/689,880, entitled Prosthetic Disorder Response Systems, which fully implanted, automatic, diagnostic, and therapeutic, can most promptly and directly treat any disorder for which a fluid counteractant can be formulated.

While improved outcomes in surgery are the object in side-entry devices as delineated in U.S. Pat. No. 11,759,186 as well as here, improved outcomes throughout the field of internal medicine is the object in prosthetic disorder response systems, delineated in U.S. Pat. Nos. 11,759,186, 11,013,858 as well as here. While the degree of automation if any involved in a given application is subject to vary, the use of these various devices under the control of the implanted automatic disorder response system is virtually inseparable in that as passive end-termini or active system end-effectors, all applications of the one will require the use of the other making these integral components of a unit mechanism.

This integrity of medical control and end-effector is further evident in the one or more accessory channels of which at least one is incorporated into every such device to allow the insertion of a drugline part number 8 or 8′ to allow medication to be directly targeted to the site, the automatic detection of the need for and control over the release of the medication entrusted to an implanted microcontroller, or in more complex disease, a microprocessor, to execute its prescription-program. Automatic implanted delivery thus also includes sensors, small drug reservoirs, and druglines.

Described below in sections 2b (1)(b), entitled Metered Compound Vascular Bypass, or Metered Switched-organ Transplantation; 2b (1)(b)(b2), entitled Prenatal Compound Vascular Bypass, or Switched-organ, Transplantation of the Heart; and 2b (1)(b)(b3), entitled Neonatal and Infant Compound Vascular Bypass, or Switched-organ, Transplantation of the Heart, the prenate and neonate born with an unsurvivably malformed heart are at the ideal time of life to have that heart replaced. In addition to the immaturity of the immune system this early in life and the hypoxia-inducible factors evolved to adapt to the hypoxia of gestation, compound vascular bypass solid organ transplantation eliminates incision into the donor and recipient hearts and is performed without the need for cardiopulmonary bypass or general anesthesia.

For these reasons, those who would appear most vulnerable are, in fact, those a considerable reduction in risk for having a severely malformed organ, to include the heart, replaced. A potential pitfall is increased susceptibility to infection; however, the measures for minimizing such an eventuality and if necessary, eradicating it, have long been well established, and the earlier a defective heart is replaced, the less will be the secondary or consequential damage to all the organs and tissues of the body, addressed below in section 2a(1), entitled Congenital Defects of the Heart and Other Organs—the Importance of Prompt Intervention (see, for example, Li, Y., Song, D., Yu, Z., Zhang, Y., Liu, Z., and Yan, T. 2024, “Effect and Mechanism of Hypoxia on Differentiation of Porcine-induced Pluripotent Stem Cells into Vascular Endothelial Cells,” In Vitro Cellular and Developmental Biology, Animal (Berlin, Germany) 60(1): 9-22; Semenza, G. L. 2023. “Hypoxia-inducible Factors: Roles in Cardiovascular Disease Progression, Prevention, and Treatment,” Online, Cardiovascular Research (Oxford, England) 119(2): 371-380; Zhao Y. Xiong W. Li C. Zhao R. Lu H. and 5 others 2023. “Hypoxia-induced Signaling in the Cardiovascular System: Pathogenesis and Therapeutic Targets,” Online, Nature Signal Transduction and Targeted Therapy (London, England) 8(1): 431; Yu, B., Wang, X., Song. Y., Xie, G., Jiao. S., and 4 others 2022. “The Role of Hypoxia-inducible Factors in Cardiovascular Diseases,” Pharmacology and Therapeutics (Oxford, England) 238:108186; Fortmann I, Dammann M T, Siller B, Humberg, A, Demmert M, and 7 others 2021. “Infants Younger Than 90 Days Admitted for Late-onset Sepsis Display a Reduced Abundance of Regulatory T Cells,” Online, Frontiers in Immunology (Lausanne, Switzerland) 12:666447; Lucero Garcia Rojas, E. Y., Villanueva, C., and Bond, R. A. 2021. “Hypoxia Inducible Factors as Central Players in the Pathogenesis and Pathophysiology of Cardiovascular Diseases,” Online, Frontiers in Cardiovascular Medicine (Lausanne, Switzerland) 8:709509; Simon, A. K., Hollander, G. A., and McMichael. A. 2015. “Evolution of the Immune System in Humans from Infancy to Old Age,” Online, Proceeding of the Royal Society. Biological Sciences (London, England) 282(1821): 20143085; Gervassi, A. L. and Horton, H. 2014. “Is Infant Immunity Actively Suppressed or Immature?,” Online, Virology: Research and Treatment (Thousand Oaks, California) 2014(5): 1-9; Ygberg, S. and Nilsson, A. 2012. “The Developing Immune System—from Foetus to Toddler,”Acta Paediatrica (Oslo, Norway) 101(2): 120-127; Ziello, J. E., Jovin, I. S., and Huang, Y. 2007. “Hypoxia-inducible Factor (HIF)-1 Regulatory Pathway and Its Potential for Therapeutic Intervention in Malignancy and Ischemia,” Online, Yale Journal of Biology and Medicine (New Haven, Connecticut) 80(2): 51-60; Delespesse, G., Yang, L. P., Ohshima, Y., Demeure, C., Shu, U., Byun, D. G., and Sarfati, M. 1998. “Maturation of Human Neonatal CD4+ and CD8+T Lymphocytes into Th1/Th2 Effectors,” Vaccine (Amsterdam, North Holland, Netherlands) 16(14-15): 1415-1419).

The ischemia-free methods to be described here include not only a novel method for solid organ transplantation but the replacement of a segment along a major vessel such as the thoracic aorta without the need to interrupt the flow of blood, this compared to the reduction in circulatory arrest time to 75 minutes using a recent technique (Pichlmaier, M., Peterss. S., Rantner, B., and Tsilimparis N. 2023. “Deceptive Safety of Unsupported Fabric Fenestrations in Hybrid Aortic Arch Replacement Using the EB-SAFER [extended branched stented anastomosis frozen elephant trunk repair] Technique,” Online, Journal of Thoracic and Cardiovascular Surgery Techniques (New York, New York) 18:22-23; Hashizume, K., Matsuoka, T., Mori, M., Takaki, H., Koizumi, K., and 4 others 2022. “Total Arch Replacement with Extended Branched Stented Anastomosis Frozen Elephant Trunk Repair for Type A Dissection Improves Operative Outcome,” Online, Journal of Thoracic and Cardiovascular Surgery Techniques (New York, New York) 17:1-9; (see also, for example, Gao, J., Yan, J., Duan, Y., Yu, J., Li, W., and 5 others 2024. “Aortic Arch Branch-prioritized Reconstruction for Type A Aortic Dissection Surgery,” Online, Frontiers in Cardiovascular Medicine (Lausanne, Switzerland) 10:1321700; Bashir, M., Mohammed, I., Al-Tawil. M., Jubouri, M., Agbobu, T., and Chen, E. P. 2023. “Frozen Elephant Trunk: The Gold Standard,” Online, Cardiovascular Diagnosis and Therapy (Hong Kong, China) 13(3): 623-627; Kong, X., Zhao, L., Pan, Z., Li, H., Wei. G., Wang, Q. 2023. “Acute Renal Injury after Aortic Arch Reconstruction with Cardiopulmonary Bypass for Children: Prediction Models by Machine Learning of a Retrospective Cohort Study,” Online, BioMed Central European Journal of Medical Research (London, England) 28(1): 499; Pitts, L., Kofler, M., Montagner, M., Heck, R., Iske, J., and 5 others 2023. “Cerebral Protection Strategies and Stroke in Surgery for Acute Type A Aortic Dissection,” Online, MDPI [Multidisciplinary Digital Publishing Institute] Journal of Clinical Medicine (Basel, Switzerland) 12(6): 2271; Shaikh, F. A., Khalil, S. I., Ander, E. H. Calvelli, H. R., Kashem, M. A., and Mokashi, S. A. 2023. “Cerebral Protection Strategies for Type A Aortic Dissection Repair,” Indian Journal of Thoracic and Cardiovascular Surgery (New Delhi, India) 39 (Suppl 2): 308-314; Takei, Y., Wu, I. H., Chan, C. Y., and Chi, N. H. 2023. “Case Report: Total Arch Replacement with a Frozen Elephant Trunk Utilizing Open Hybrid in-Situ Fenestration Technique for Thoracic Aortic Arch Aneurysm,” Online, Frontiers in Surgery (Lausanne, Switzerland) 10:1224013; Tang, Z. and Shao, Y. 2023. “Postoperative Thrombocytopenia and Subsequent Consequences in Acute Type A Aortic Dissection,” Online, Annals of Medicine (London, England) 55(2): 2281653; Villani. R., Loizzi, D., Sacco, A. F., Mirabella, L., Santoliquido, M., and 3 others 2023. “Prevalence and Clinical Relevance of Liver Dysfunction after Thoracic Surgery: A Retrospective Study,” Online, Nature. Scientific Reports (London, England) 13(1): 23045; Bolognesi. R., Tsialtas, D., Bolognesi, M. G., Assimopoulos, S., Azzarone, M., and Volpi, R., 2022. “Perioperative Complications following Major Vascular Surgery. Correlations with Preoperative Clinical, Electrocardiogram, and Echocardiographic Features,” Online, Acta Bio-medica: Atenei Parmensis (Parma, Italy) 93(3): e2022255; Fang, Z., Wang, G., Liu, Q., Zhou, H., Zhou, S., and 9 others 2019. “Moderate and Deep Hypothermic Circulatory Arrest Has a Comparable Effect on Acute Kidney Injury after Total Arch Replacement with Frozen Elephant Trunk Procedure in Type A Aortic Dissection,” Online, Interactive Cardiovascular and Thoracic Surgery (Oxford, England) 29(1): 130-136; Nadim M K, Fomi L G, Bihorac, A. Hobson C. Koyner J L, and 22 others 2018. “Cardiac and Vascular Surgery-associated Acute Kidney Injury: The 20th International Consensus Conference of the ADQI (Acute Disease Quality Initiative) Group,” Online, Journal of the American Heart Association (Oxford, England) 7(11): e008834; Fernandez Suarez, F. E., Fernández Del Valle, D., González Alvarez, A., and Pérez-Lozano, B. 2017. “Intraoperative Care for Aortic Surgery Using Circulatory Arrest,” Online, Journal of Thoracic Disease (Hong Kong, China) 9 (Supplement 6): S508-S520; Pasin, L., Nardelli, P., Belletti, A., Greco, M., Landoni, G., and 3 others 2017. “Pulmonary Complications after Open Abdominal Aortic Surgery: A Systematic Review and Meta-analysis,” Journal of Cardiothoracic and Vascular Anesthesia (Philadelphia, Pennsylvania) 31(2): 562-568; Sabzi, F. and Faraji. R. 2015. “Liver Function Tests following Open Cardiac Surgery,” Online, Journal of Cardiovascular and Thoracic Research (Tabriz, Iran) 7(2): 49-54).

Inserted subcutaneously in the pectoral region with lines routed to their destinations subcutaneously with the aid of ultrasound, or echocardiography, and positioned laparoscopically with associated instruments through small ‘keyhole’ incisions, the implantation of such an automatic response system involves nothing to recoil over despite being minimally intrusive—this because the benefits fundamentally outweigh the few, unlikely, and readily counteracted complications possible. Usually placed in an open surgical field required for other reasons, side-entry valves and servovalves call for the use of the human hand; however, with training this can be accomplished through small incisions.

Vascular side-entry devices which can redirect or divert the flow of blood and give direct access to the lumen for the release of drugs are placed without interruption in the flow of blood through the substrate vessel. This one factor is distinguishing and has far reaching medical and surgical significance. The need to use cardiopulmonary bypass and general anesthesia in open heart surgery or heart transplantation to sustain circulation can prove a source of complications both numerous and serious. To avoid the use of these, it is essential that both circulations remain uninterrupted through the heart as well as through the systemic and pulmonary circulation of both donor and recipient.

In order to perform a compound vascular heart transplant, the donor must have been kept on life support initiated prior to and intentionally sustained past death and must be positioned alongside the recipient. Not currently in effect, the subsection below entitled Deceased Donor Sustainment Centers described below in this section explains how this is to be accomplished. In eliminating preoperative harvesting of the donor organ, its subsequent anoxia, and transection prior to transplantation, compound vascular bypass organ transplantation reduces surgical trauma and the ischemia-reperfusion injury strongly suspected to play a part in jeopardizing long-term graft durability due to late-term cardiac allograft vasculopathy and/or cancer.

The direct seamless transfer of a donor organ from the circulatory system of the donor into that of the recipient with no interruption in the circulation of either requires first, that the donor and recipient be placed side by side and second, that a means for achieving uninterrupted blood flow between the two be available. Cardiac allograft vasculopathy likely a late-term consequence of immune aversion, ischemia during the transplantation process appears to bolster its development.

The primary capability imparted by side-entry jackets and vascular valves is to make possible direct access to the lumen of a tubular anatomical structure, or ductus, most often a blood vessel, without interrupting the flow of blood therethrough, and to make uninterrupted flow possible precludes transecting the ductus. The realization of a temporary combining of two circulatory systems with no interruption in blood flow, referred to as reciprocal cross-circulation, requires that no vessel be transected. Instead, each must be connected at the side to an outflow or inflow tube or catheter leading to a corresponding connection at the other end.

The paramount object in devising side-entry devices is to impart the ability to draw off all or a continuously variable fraction or the totality of the flow through a ductus, most often a blood vessel, for continued flow through a synthetic line without the need to transect and thereby eliminate the need to interrupt flow through the ductus for more than a brief interval, in itself a major cause for complications due to ischemia-reperfusion injury in cardiovascular operations. The implementation of outflow with another valve, for example, is usually antipodal in mechanism in relation to the same ductus in the recipient.

The only way to accomplish this is to enter through an opening in the side of the ductus with an extendable and retractable tap-tongue diversion chute, part number 18 in the drawing figures, having a flow diverting distal tip at the front end curved in the direction opposite to the flow. Using linear plunger solenoid-driven valves, control of the diversion chute is bistable, that is, from fully open or fully closed to the opposite state as needed to perform a compound vascular bypass sudden switch solid organ transplant and as preferred for the same operation in a prenate, neonate, or young toddler, or continuously variable to perform a metered transplant. This connotes that some degree of protrusion outside the ductus will be unavoidable.

Connection thus demands a means for perforating the side of each vessel of the graft organ in either party to create an ostium to allow outflow or inflow through a catheteric bloodline led to the other party with as little loss of blood, lymph, glandular contents, or intestinal contents as possible. To do this, a selectably positionable cushion-lined collar containing a diversion chute is placed in perivascular relation to the preferred level and desired angle along each substrate vessel. In a simple side-entry jacket as described in U.S. Pat. No. 11,759,186, entitled Ductus Side-entry Jackets and Prosthetic Disorder Response Systems, the ostium is created through the application of suction generated by an aspirator through a connecting catheter inserted in the back of the jacket and connected to a reciprocating collar having a razor-sharp trepan, or trephine, leading edge. The vacuum incises and draws a plug of tissue out of the side of the ductus to create the ostium.

In this way, the vacuum pressure is used to draw the wall outward across a trepan advancing from the jacket until it reaches the endothelium. This action cuts a plug out of the side of the vessel while pressurized water emitted from around the trepan is directed against the ostium to restrain blood from escaping. The plug is then extracted with the aid of a hook-ended guidewire, and the catheter is connected to the receiving vessel through the same action. A simple flow diversion side-entry jackets as described below in section 2b, entitled Vascular Valves and Servovalves in the Diversion of Blood Flow, in the subsection thereof entitled Simple Flow Diversion Side-entry Jackets or flow directional guides, shown in FIGS. 10F and 10G can be used to divert blood from an end-arterial vessel such as a coronary artery or from one vessel into another at the unchangeable fixed rate set by the heart. In contrast, a vascular valve or servovalve has a flow diversion chute which can be extended or retracted into the column passing through a blood vessel, for example, to control the volume of blood diverted.

When only bistable or flow/no flow control is needed, the valve is driven by a highly damped nonsparking plunger solenoid such as those shown in FIGS. 7 and 8 as part number 32; if the diversion chute must be capable of tapping off a graduated and continuously variable volume of blood flow from or into the substrate vessel, that is, necessarily capable of outflow or inflow from 0 to 100 percent and every gradation in between, servomotor driven servovalves are. needed. The nonsparking feature is imperative to prevent fires (see, for example, Stormont, G., Anand, S., and Deibert, C. M. 2023. “Surgical Fire Safety,” Online, Treasure Island, Florida: StatPearls Publishing Co.; Mai, C. L., Wongsirimeteekul, P., Petrusa, E., Minehart, R, Hemingway M, and 3 others 2020. “Prevention and Management of Operating Room Fire: An Interprofessional Operating Room Team Simulation Case, Online MedEdPORTAL (Washington, DC) 16:10871). Damping is imperative to eliminate the knocking, noise, and impact of plunger solenoids used in machinery intolerable in an implant.

Unlike passive side-entry jackets which, as indicated, support a fixed volume of the blood tapped off, vascular valves and servovalves require an obturator fixed to the front tip of the diversion chute so that when the diversion chute is fully retracted, flow is restricted to the substrate vessel. Outflow is then controlled by extension of the diversion chute into the lumen of the substrate vessel to the extent corresponding to the volume of blood to be tapped off into another vessel. Controlled by the implanted sensor-responsive microcontroller, or in the case of comorbid disease, a master control microprocessor, this action is continuously variable in immediate response to the condition or conditions detected.

The obturator also serves to more strongly distinguish and direct the stream to be diverted from that to continue through the vessel. The need for an obturator at the front end of the diversion chute in a vascular servovalve does not allow for the placement of an extendable trepan, hence, the extraction of a plug from the side of a vessel as in the placement of a simple side-entry jacket. Instead, plug extraction is accomplished by touching the razor sharp ferruled front end of a catheter connected to an aspirator to the side of the vessel and drawing the plug out by suction. The plug seen through the transparent catheter, the operator instantly shifts his thumb from just beside the ostium to seal off the ostium, while the other hand is used to snap on the valve or servovalve.

All side-entry and related devices to include nonjacketing side-entry connectors, described in U.S. Pat. No. 11,013,858, entitled Nonjacketing Side-entry Connectors and Prosthetic Disorder Response Systems, and confluence conduits, ‘tuned’ shunts, and bloodline switches as described below incorporate at least one accessory channel giving access to the interior of the device and the substrate lumen to which the device is connected. An accessory channel allows the insertion of a drugline connected to a drug reservoir usually implanted subcutaneously in the pectoral region, of which the outlet is controlled by the implanted disorder response system.

Vascular valves and servovalves adapted from ductus side-entry jackets, the terminology used to denote the different type lines used was originally provided in U.S. Pat. No. 11,759,186, entitled Ductus Side-entry Jackets and Prosthetic Disorder Response Systems. As used here the terms adaxial and abaxial are anatomical, not botanical, adaxial meaning toward, and abaxial meaning away from, the central longitudinal axis, generally that running through the center of a blood vessel or other ductus.

Plural drug reservoirs almost always necessary, each or a combination of reservoirs is made to release medication into the side-entry device and ductus to which it is connected in response to commands from the system controller based upon sensor inputs in accordance with its prescription-program. The outflow or inflow lumen of the device referred to as the bloodline or mainline, this allows the insertion of and release of medication through a tube or catheteric drugline, or sideline into the device and the lumen to which it is connected.

The release of medication directly pipe-targeted thus to a selectably positional level along a vessel, duct, or the gut, for example, which bypasses the levels farther upstream thereof, is entrusted to a fully implanted automatic disorder response system of the kind described in U.S. Pat. No. 11,759,186, entitled Ductus Side-entry Jackets and Prosthetic Disorder Response Systems, and copending application Ser. No. 17/689,880, entitled Prosthetic Disorder Response Systems. If only fixed side-entry jackets are used to create a femorofemoral shunt, for example, control will usually be entrusted to a microcontroller implanted in the pectoral region to allow the directly targeted release of medication to the junctions.

Any patient with a serious chronic or long-term episodic condition not adequately controlled with oral medication or self-administered injection would benefit from the placement of a fully implanted automatic disorder response system which equipped with the right sensors to instantly detect the inception of symptoms and programmed to automatically pipeline-target drugs such as an anticoagulant, anti-inflammatory, and antimicrobial, directly to the site or sites calling for treatment. Provided the medicinal means exist to suppress the condition, such a system is able to counteract symptoms before the patient even becomes aware of these. The facts, however, can be telemetrically transmitted to the clinic for review by the medical staff.

Positioning a simple side-entry jacket just upstream to the direct end-to-side anastomosis allows taking advantage of the direct medication targeting capability of the connecting device. In comorbid disease, control is entrusted to a master control microprocessor similarly positioned with fixed side-entry jackets used to release the medication. In more complex applications, such as in a compound vascular heart transplant where the release of drugs must be coordinate with the control of blood flow, servovalves must be used.

The master control microprocessor is fed inputs from symptom-attuned sensors situated at the sites of disease or the intersections pertinent to the action indicated and is programmed to effect the responsive measures as a hierarchical control system in which a different arm or branch of control is assigned to the different organs, organ systems, diseases, or nidi of disease in accordance with its prescription-program. In this way, the different arms of control are coordinated to target medication and control the fine adjustment of the vascular servovalves in achieving the optimal state of homeostasis across the sum of morbidities for the specific patient in which artificial intelligence, briefly addressed below in this section, subsection entitled n. Adverse Reactions to Drugs, will play a part.

That side-entry jackets and servovalves can be combined in different applications is considered obvious. Additional summary information is provided below in section 3, entitled Summary of the Invention A prosthetic disorder response system is a fully implanted network of sensors, drug reservoirs, direct—to target drug delivery lines, shunts, specialized connectors, and other components such as electrostimulatory neuromodulators, provided to assist the native autonomic, immune, endocrine, metabolic, and biochemically implemented integrated stress response and crisis response-initiating adaptive pathway-all-encompassing sensory and motor function—in responding or more effectively responding to conditions essential to maintain optimal health in the patient.

Line and line connector patency critical for maintaining the functional sufficiency of a prosthetic disorder control system, multiple measures are provided to preclude and counteract obstruction. All blood-conveying, or bloodlines, and drug-conveying, or druglines, and line connection devices are made of polymeric materials or are coated over their external surfaces with surface treatments specifically devised to repel and thus prevent adhesions and accretions along the internal surfaces of substances deposited out of the fluid transmitted. While the traveling waves of the pulse at the periphery of an artery are suppressed when passing through a catheteric bloodline, this action is resumed once the flow passes into another native artery.

As shown in FIGS. 2, 5, 7, 8, and 10A, in addition to drugline 8′ which allows the substrate adventitia to be wetted with a topical counterirritant to run down around sidestem 19 and could be complemented by a similar drugline on the opposite side of the substrate vessel, drugline 8 with outlet pore 31 at the underside tip of diversion chute 18 releases drugs into the lumen of the substrate vessel. At less degrees of extension than completely as shown, undiverted blood would continue to flow down through lumen 1 to be medicated thus. At full diversion, one or more druglines (not shown) would be used to release medication directly into the lumen of sidestem 19 and therefore into the bloodline, or mainline, connected to the sidestem. A drugline just behind 8′, for example, would continue entirely into the lumen of sidestem 19.

Confluence conduits are addressed below in section 2b(1)(b)(b14), entitled Confluence Conduits: Connection of One or More Bloodlines to Flow through a Common Channel to the Same or a Different Number of Bloodlines with Self-leveling Apportionment of Outflow. In FIGS. 35A and 35B, a no flow interruption inlet connector between an organic and a synthetic ductus can be a manually operated flow diversion valve such as that shown in FIG. 2 or a solenoid-driven bistable valve such as those shown in FIGS. 7 and 8. Alternatively, the inlet into a confluence conduit can be a continuously variable servovalve as shown in FIG. 10A. This is why FIGS. 35A and 35B, depict the connectors are depicted with only the diversion chute, part number 18. With multiple confluence conduits, those manually operated are switched by the members of the operating team synchronizing their action; solenoid-driven valves, are best actuated from the same switch.

For heart transplantation in a late prenate, neonate, or small toddler where the vessels are tiny in diameter, as shown in FIG. 16 include 9 vessels each of which must be connected at either end for a total of eighteen connectors unless the supraaortic vessels are also to be separately connected to the confluence conduit with costly microminiature valves, taking up much time, with a patient already impaired, the intricacy and skill level required recommends avoiding the need to separately make each of these connections. This is accomplished by means of the device shown in FIG. 10G and addressed below toward the end of section 2b(1)(b)(b14) entitled Confluence Conduits: Connection of One or More Bloodlines to Flow through a Common Channel to the Same or a Different Number of Bloodlines with Self-leveling Apportionment of Outflow, in the subsection thereof entitled Tiny Vessel Grouped Inlet/Outlet Expandable Confluence Conduits.

Druglines with direct outlet into the lumen of the substrate lumen are schematically indicated as part number 8 in FIG. 35A depicting a confluence conduit and FIG. 35B depicting a switchable confluence conduit. While FIGS. 35A and 35B represent the extension arms as integral (unitary, continuous) with the synthetic body of the conduits as equal in length, conduits can be provided with integral extension arms that differ in length. This allows confluence among vessels which to be brought together would otherwise necessitate inordinate time or dissection to inlet or outlet into the conduit.

Because of its inherent cross-leveling of outflow, provided stenotic or atherosclerotic segments have been remediated, a confluence conduit such as shown in FIG. 35A as positioned along two input and three outlet vessels can accommodate supernumerary anomalies of vasculature as is sometimes encountered in kidney transplantation. Input and output valves or servovalves are connected along the length of the input vessels and to the extension arms unitary with the body of the confluence conduit so that in FIG. 35A, for example, vessel 194 with take-off passive one-way flow guide or valve 195 as tap-off outflows into conduit extension arm 198.

Confluence conduits provide several advantages over the need to trace individual vessels separately where these are numerous as in a heart transplant. Vessels channeled conjointly thus can be differentially treated as a group medicinally by means of a drugline or adjusted in flow rate with the aid of an inline axial pump. The drawing figures are schematic in omitting the bloodline insertion joints into the conduit but only when the bloodlines are directly connected to the conduit rather than integral arms extending from it as illustrated. A confluence conduit usually has a luminal, or flow-through, volume delivery rate in milliliters per second equal to the sum of the volume delivery rates of the bloodlines connected to it.

Exceptionally, the volume delivery can be passively adjusted by producing the conduit with a somewhat increased or decreased internal diameter to deviate from the sum of the volume delivery rates of the bloodlines connected to it. The volumetric flow-through rate can also be adjusted by introducing a constriction or dilation along the length of the conduit. When an outlet into a bloodline would be better positioned along the length rather than the terminus of the conduit itself, the volumetric flow-through rate into that outlet can be adjusted by using a conduit with such a constriction or dilation upstream to the outlet. FIGS. 35A and 35B show confluence conduits wherein a reversible inline axial pump is positioned along the length of the conduit to accelerate or decelerate the volumetric flow-through rate.

Druglines that outlet directly into the lumen of a bloodline or confluence conduits must incorporate measures to prevent blood from reverse flow into the outlet of the drugline where clot would accumulate. Thrombus repellent materials are addressed shortly below in the subsection entitled Materials and Surface Treatments to Repel Thrombus. By providing a separate internal layer of the non-stick coating polytetrafluoroethylene, and an outer layer of a pliant polymer to prevent kinking, or by vapor deposition of a non-stick fluoropolymer coating along the internal wall of the lumen, pliant druglines can eschew clot, which purpose can be furthered through the occasional automatic release of a drop of a heparin by the system controller.

Alternatively, druglines for the release of a single drug can be prefilled with a formulation of the drug as a fluid with sufficient viscosity to prevent the entry of blood. A nominally single drug reservoir with a miniature inline reversible axial pump at the outlet can proper the drug forward into the side-entry device and its substate vessel or withdraw that filling the line back up into the reservoir to allow another drug to be used. For use thus, the drugline must incorporate a nonstick internal coating or layer to minimize retention of the earlier drug along the lumen wall.

An object to provide life-sustaining prosthetic components in an infant that will spontaneously adjust to the size of the patient and thereafter expand in step with growth thereby averting the need for reentry and revision, all conduits-mainlines, or bloodlines, sidelines, or druglines, tie-lines, and confluence conduits, must be mechanically devised to restrainedly expand in response to the average pressure exerted on the internal walls of the conduit and must be made of materials or surface coated to repel clot and biofilm.

The use of elastomers such as spandex is discounted as increasing in resistance to further stretching the more the stretching continues, and pleating in some form, whether accordion, Watteau, box, cartridge, fluted, honeycomb, or some combination of these, pleating and the accretion of thrombus are directly related in that the reentrant into every pleat will attract thrombus much as does the cavity of a saccular aneurysm in the abdominal aorta or carotid artery, for example (see, for example, Bontekoe, J., Matsumura, J., and Liu, B. 2023. “Thrombosis in the Pathogenesis of Abdominal Aortic Aneurysm,” Online, Journal of Vascular Science (New York, New York) 4:100106; Faluk; M. and De Jesus, O. 2022. Saccular Aneurysm,” Online, Treasure Island, Florida: StatPearls Publishing Co.; Yoshihara, R., Shindo, K., Ogino, T., and Nakamura, H. 2022. “Acute Middle Cerebral Artery Occlusion Caused by Spontaneous Thrombosis of a Small Internal Carotid Artery Aneurysm: Illustrative Case,” Online, Journal of Neurosurgery Case Lessons (Charlottesville, Virginia) 4(18): CASE22335; Ngoepe, M. N., Frangi, A. F., Byrne, J. V., and Ventikos, Y. 2018. “Thrombus in Cerebral Aneurysms and the Computational Modeling Thereof: A Review,” Online, Frontiers in Physiology (Lausanne, Switzerland) 9:306).

The vascular prostheses described herein do not stretch but rather open to a larger size, various materials, surface treatments made possible because the dimensions of the surface do not stretch, and agents available to deter pathogens and the accretion of clot. The terms ‘vault’ and ‘expansion vault’ in this context are meant to denote circumferential arcuate, parabolic, or semicircular abaxial projections which extend outward from the longitudinal axis of the prosthesis separated by adaxial flat sections. As shown in FIGS. 15C, 15D, 36A, and 36F, the alternating flats 233 and circumferential expansion vaults 234 of the prosthesis wall effectuate expansion not through stretching with increasing compression but rather through gradually opening and thus presenting a continuous surface amenable to coating.

The conduits shown in FIGS. 15C, 15D, and 36F incorporate flats 233 and vaults 234 expandable to accommodate growth in a prenate, neonate, or infant intended to support a compound vascular bypass heart transplant where to valve each of the tiny vessels would significantly increase the duration of the procedure for a patient already frail. The degree of reduction in trauma using the compound bypass method will often be the factor that makes a heart transplant feasible and will certainly avert the numerous serious complications that either reconstructive surgery or a conventional heart transplant would impose to reduce the likelihood for a favorable outcome.

Suitable materials are 2.0 millimeter thick injection blow molded polypropylene or polyetheretherketone, good resorption resistors, with side extensions to produce branches. To assure the use of magnetic resonance imaging in the future, all parts are made of nonferrous materials. The completed part is then surface treated as will most effectively protect it from biofilm and clot. Vapor deposited oxygen plasma modified parylene or gold, for example, are effective to repel bacterial colonization and biofilm (Hao, D., Lin, J., Liu, R., Pivetti, C., Yamashiro, K., and 9 others 2023. “A Bio-instructive Parylene-based Conformal Coating Suppresses Thrombosis and Intimal Hyperplasia of Implantable Vascular Devices,” Online, Bioactive Materials (Beijing, China) 28:467-479; El-Chami, M. F., Mayotte, J., Bonner, M., Holbrook, R., Stromberg, K., and Sohail, M/R. 2020. “Reduced Bacterial Adhesion with Parylene Coating: Potential Implications for Micra Transcatheter Pacemakers,” Journal of Cardiovascular Electrophysiology 31(3): 712-717; Golda-Cepa, M., Riedlova, K., Kulig, W., Cwiklik, L, and Kotarba, A. 2020. “Functionalization of the Parylene C Surface Enhances the Nucleation of Calcium Phosphate: Combined Experimental and Molecular Dynamics Simulations Approach,” Online, American Chemical Society Applied Materials and Interfaces (Washington, D. C.) 12(11): 12426-12435).

Clot can also be thwarted when surfaces in contact with blood are suitably coated (Brancato, L., Decrop, D., Lammertyn, J., and Puers, R. 2018. “Surface Nanostructuring of Parylene-C Coating for Blood Contacting Implants,” Online, MDPI [Multidisciplinary Digital Publishing Institute] Materials (Basel, Switzerland) 11(7): 1109).

Treatment of the internal surface if not coatable is the dispensation of appropriate drips by the implanted prosthetic disorder response system. This not only eliminates the problems of increased resistance to further stretching in proportion to the degree of stretching already applied as would the use of spandex, and an excessive propensity for accumulating clot as would pleats, but because the continuity of the surface is not subject to stretching, makes it possible to sputter or vapor deposit various coatings such as parylene or gold onto the wall. Polypropylene (PP) or polyetheretherketone (PEEK) are preferred for resistance to resorption.

The increase in pressure with growth presses outward against the flats causing the expansion vaults to gradually open, thus increasing both the diameter and length of the lumen. While the vault reentrants induce some turbulent (as opposed to laminar) flow where these are continuous with the flats, shear stress does not arise and cannot produce in a synthetic-polypropylene of polyetheretherketone-‘vessel’ the degradative consequences these would have in an innate, or for that matter, a tissue engineered vessel that was at fault in this regard.

Turbulent flow promotes breakdown to include the formation of atheromatous plaques, pseudoaneurysms, aneurysms, and dissections which, in addition to the more immediate problems these cause, also produce turbulent flow. Until perfected, patches, endoluminal repairs, and tissue engineered vessels will remain susceptible to such problems. By the same token, tissue engineered vessels which implanted in a child would grow will eventually be preferred.

Organic lesions such as stenoses, aneurysmal, dissectional, and malfunctioning valves also cause turbulent flow where the degradative consequences can be significant (see, for example, Matienzo, D. and Bordoni, B. 2023. “Anatomy, Blood Flow,” section on Blood Flow and Lymphatics, Online, Treasure Island, Florida: StatPearls Publishing Co.; Zhuang, B., Sirajuddin, A., Zhao, S., and Lu, M. 2021. “The Role of 4D Flow MRI for Clinical Applications in Cardiovascular Disease: Current Status and Future Perspectives,” Online, Quantitative Imaging in Medicine and Surgery 11(9): 4193-4210; Chaturvedi, A., Hamilton-Craig, C., Cawley, P. J., Mitsumori, L. M., Otto, C. M., and Maki, J. H. 2016. “Quantitating Aortic Regurgitation by Cardiovascular Magnetic Resonance: Significant Variations Due to Slice Location and Breath Holding,” European Radiology 26(9): 3180-3189; Bogren, H. G. and Buonocore, M. H. 1999. “Complex Flow Patterns in the Great Vessels: A Review,” International Journal of Cardiac Imaging, 15(2): 105-113).

In bloodlines, such an accumulation of adherent material, or accretant, usually consists of clot and/or a biofilm; in urine-passing lines such as shown in FIGS. 28 and 30, this is crystal, most often consisting of calcium and oxalate, or cystine, uric acid, or struvite. Base materials suitable for side-entry devices, vascular valves, and polymeric catheteric conduits include all the usual polymers and metals used to make implants, both internal and external surfaces treated. Usually implanted for life, durability is the cardinal consideration as to which method of treatment should be used.

Vascular side-entry devices always incorporate one or more accessory channels into which a fluid substance counteractant can be directly pipe-targeted into the device and its line. Further improvements in assuring unobstructed flow through tiny catheters as bloodlines and druglines will almost certainly mimic the means that afford this facility used by nature to allow the smallest vessels such as those supplying and draining the orbits and labia in neonates.

Of the two ways the occlusion and fouling of bloodline lumina is counteracted, the first, passive, is the use of lines made of or coated with intrinsically antithrombotic and/or antimicrobial materials. The other, active, is the intermittent release of an antithrombotic drip into the flow as detected by sensor implants addressed below in this section (Barriuso, I., Worner, F., and Vilahur, G. 2022. “Novel Antithrombotic Agents in Ischemic Cardiovascular Disease: Progress in the Search for the Optimal Treatment,” Online, MDPI [Multidisciplinary Digital Publishing Institute] Journal of Cardiovascular Development and Disease (Basel, Switzerland) 9(11): 397; Fuentes, E., 2022. “Modulation of Glycoprotein VI and Its Downstream Signaling Pathways as an Antiplatelet Target,” Online, MDPI [Multidisciplinary Digital Publishing Institute] International Journal of Molecular Sciences (Basel, Switzerland) 23(17): 9882).

Some materials are intrinsically or can be made thrombus-, bacteria-, and sometimes, fatigue-resistant as well (see, for example, Abdollahi, S., Stephens, E. D., Uy, M. A., Fatehi Hassanabad, A. Fedak, P. W. M., and Bady, M. 2023. “Super-repellent and Flexible Lubricant-infused Bacterial Nanocellulose Membranes with Superior Antithrombotic, Antibacterial, and Fatigue Resistance Properties,” [American Chemical Society] Applied Materials and Interfaces 15(22): 26417-26430; Kasapgil, E., Bady, M., Cantu, C. A., Rahmani, S., Erbil, H. Y., and 4 others 2021. “Polysiloxane Nanofilaments Infused with Silicone Oil Prevent Bacterial Adhesion and Suppress Thrombosis on Intranasal Splints,” [American Chemical Society] Biomaterials Science and Engineering 7(2): 541-552; Ozkan, E., Mondal, A., Singha. P., Douglass, M. Hopkins, S. P., and 5 others 2020. “Fabrication of Bacteria- and Blood-repellent Superhydrophobic Polyurethane Sponge Materials,” [American Chemical Society] Applied Materials and Interfaces 12(46): 51160-51173).

### Materials and Surface Treatment to Repel Thrombus

Numerous materials have been adopted for their clot repellent properties or have been devised to repel thrombus (see, for example, Hao. D., Lin, J., Liu, R., Pivetti, C., Yamashiro, K., and 9 others 2023. Op cit. shortly above in this section; Douglass, M., Garren, M., Devine. R., Mondal. A., and Handa, H. 2022. “Bio-inspired Hemocompatible Surface Modifications for Biomedical Applications,” Progress in Materials Science 130:100997; Ozkan, E., Mondal, A., Douglass, M., Hopkins, S. P., Garren, M., and 6 others 2022. “Bioinspired Ultra-low Fouling Coatings on Medical Devices to Prevent Device-associated Infections and Thrombosis,” Online, Journal of Colloid and Interface Science (Orlando, Florida) 608 (Part 1): 1015-1024; Manivasagam, V. K., Sabino, R. M., Kantam, P., and Popat, K. C. 2021. “Surface Modification Strategies to Improve Titanium Hemocompatibility: A Comprehensive Review,” Online, Materials Advances (Cambridge, England) 2(18): 5824-5842; Chou, S. F., Caltrider, B. A., Azghani, A., and Neuenschwander, P. F. 2020. “Inhibition of Platelet Adhesion from Surface Modified Polyurethane Membranes,” Online, Biomedical Journal of Scientific and Technical Research (New York, New York) 32(3): 24988-24993; Krueger, I., Gremer, L., Mangels. L, Klier, M., Jurk, K., and 4 others 2020. “Reelin Amplifies Glycoprotein VI Activation and Alphallb Beta 3 Integrin Outside—in Signaling via PLC Gamma 2 [1-phospahtidylinositol-4,5-bisphosphate phosphodiesterase gamma-2)] and Rho GTPases,” Online, [American Heart Association] Arteriosclerosis, Thrombosis, and Vascular Biology (Baltimore, Maryland) 40(10): 2391-2403; Wilson, A. C., Neuenschwander, P. F., and Chou, S. F. 2019. “Engineering Approaches to Prevent Blood Clotting from Medical Implants,” Online, Archives in Biomedical Engineering and Biotechnology (San Francisco, California) 1(2): 000510; Jokinen V., Kankuri, E., Hoshian, S., Franssila, S., and Ras, R. H. A. 2018. “Superhydrophobic Blood-repellent Surfaces,” Online, Advanced Materials 30(24):e1705104; Miyachi, H., Reinhardt, J. W., Otsuru, S., Tara, S., Nakayama, H., and 7 others 2018. “Bone Marrow-derived Mononuclear Cell Seeded Bioresorbable Vascular Graft Improves Acute Graft Patency by Inhibiting Thrombus Formation via Platelet Adhesion,” Online, International Journal of Cardiology (Amsterdam, North Holland, Netherlands) 266:61-66; Leslie, D. C., Waterhouse, A., Berthet, J. B., Valentin, T. M., Watters. A. L., and 16 others 2014. “A Bioinspired Omniphobic Surface Coating on Medical Devices Prevents Thrombosis and Biofouling,” Nature Biotechnology 32(11): 1134-1140).

### Materials and Surface Treatment to Repel Microbial Colonization

And numerous materials and surface treatments have been devised to repel bacteria; however, despite the fact that the immediately postoperative period is that most susceptible to infection, increased periods of pathogenicity preceding tissue integration or the need to initiate a systemic antibiotic drip should be developed (see, for example, Duarte-Pena, L., Magana, H., and Bucio, E. 2023. “Catheters with Dual-antimicrobial Properties by Gamma Radiation-induced Grafting,” Online, MDPI [Multidisciplinary Digital Publishing Institute] Pharmaceutics (Basel, Switzerland) 15(3): 960; Garren, M., Ashcraft. M., Crowley, D., Brisbois, E. J., and Handa, H. 2023. “Derivatization of Graphene Oxide Nanosheets with Tunable Nitric Oxide Release for Antibacterial Biomaterials,” Journal of Biomedical Materials Research Part A. 111(4): 451-464; Inchingolo, A. M., Malcangi, G., Ferrante, L. Del Vecchio, G., Viapiano, F. and 8 others 2023. “Surface Coating of Dental Implants: A Review,” Online, MDPI [Multidisciplinary Digital Publishing Institute] International Journal of Functional Biomaterials (Basel, Switzerland) 14(5): 287; Ramezani. M., Labour, E. E., Ji. J., Vakil. A. U., Du. C., and 3 others 2023. “Self-Defensive Antimicrobial Shape Memory Polyurethanes with Honey-based Compounds,” Online, American Chemical Society Applied Materials and Interfaces (Washington, D. C.) 15(49): 56733-56748; Rajaramon, S., David, H., Sajeevan, A., Shanmugam, K., Sriramulu, H., Dandela, R., and Solomon, A. P. 2023. “Multi- functional Approach in the Design of Smart Surfaces to Mitigate Bacterial Infections: A Review,” Online, Frontiers in Cellular and Infection Microbiology (Lausanne, Switzerland) 13:1139026; Skerlavaj. B. and Boix-Lemonche, G. 2023. “The Potential of Surface-immobilized Antimicrobial Peptides for the Enhancement of Orthopaedic Medical Devices: A Review,” Online, MDPI [Multidisciplinary Digital Publishing Institute] Antibiotics (Basel, Switzerland) 12(2): 211; Browne, K., Kuppusamy, R., Chen, R., Wilcox, M. D. P., Walsh, W. R., Black, D. S., and Kumar, N. 2022. “Bioinspired Polydopamine Coatings Facilitate Attachment of Antimicrobial Peptidomimetics with Broad-spectrum Antibacterial Activity,” Online, MDPI [Multidisciplinary Digital Publishing Institute] International Journal of Molecular Sciences (Basel, Switzerland) 23(6): 2952; Cao, H., Qiso, S., Qin, H., and Jandt, K. D. 2022. “Antibacterial Designs for Implantable Medical Devices: Evolutions and Challenges,” Online, MDPI [Multidisciplinary Digital Publishing Institute] International Journal of Functional Biomaterials (Basel, Switzerland) 13(3): 86; Ghosh, S., Mukherjee, S., Patra, D. and Haldar, J. 2022. “Polymeric Biomaterials for Prevention and Therapeutic Intervention of Microbial Infections,” [American Chemical Society] Biomacromolecules 23(3): 592-608; Negut. I., Bita. B. and Groza, A. 2022. “Polymeric Coatings and Antimicrobial Peptides as Efficient Systems for Treating Implantable Medical Devices-associated Infections,” Online, MDPI [Multidisciplinary Digital Publishing Institute] Polymers (Basel, Switzerland) 14(8): 1611; Srimaneepong. V., Skallevold, H. E., Khurshid, Z., Zafar, M. S., Rokaya, D. and Sapkota, J. 2022. “Graphene for Antimicrobial and Coating Application,” MDPI [Multidisciplinary Digital Publishing Institute] International Journal of Molecular Sciences (Basel, Switzerland) 23(1): 499; Yang, Y., Jiang, X. Lai, H., and Zhang, X. 2022. “Smart Bacteria-responsive Drug Delivery Systems in Medical Implants,” Online, MDPI [Multidisciplinary Digital Publishing Institute] International Journal of Functional Biomaterials (Basel, Switzerland) 13(4): 173; Yu, J., Zhou, M., Zhang, L., and Wei. H. 2022. “Antibacterial Adhesion Strategy for Dental Titanium Implant Surfaces: From Mechanisms to Application,” Online, MDPI [Multidisciplinary Digital Publishing Institute] International Journal of Functional Biomaterials (Basel, Switzerland) 13(4): 169; Pandit, S., Gaska, K., Kadar, R., and Mijakovic, I. 2021. “Graphene-based Antimicrobial Biomedical Surfaces,” Online, Chemphyschem (Weinheim, Germany) 22(3): 250-263; Qiu, H., Si, Z. Luo, Y., Feng. P., Wu, X., and 5 others 2020. “The Mechanisms and the Applications of Antibacterial Polymers in Surface Modification on Medical Devices,” Online, Frontiers in Bioengineering and Biotechnology (Lausanne, Switzerland) 8:910; Li, X., Qi, M., Sun, X., Weir, M. D., Tay, F. R., and 5 others 2019. “Surface Treatments on Titanium Implants via Nanostructured Ceria for Antimicrobial and Anti-inflammatory Capabilities,” Acta Biomaterialia 94:627-643; Rai. A., Pinto, S., Evangelista, M. B., Gil, H., Kallip, S., Ferreira, M. G., and Rerreira, L. 2016. “High-density Antimicrobial Peptide Coating with Broad Activity and Low Cytotoxicity against Human Cells,” Acta Biomaterialia 33:64-77).

To spontaneously adjust in response to growth, blood and druglines, vascular prostheses, and confluence conduits in pediatric patients incorporate abaxially domed segments, or expansion vaults 234, which alternate with adaxial flats 233 as shown in FIGS. 15C, 15D, 36A and 36F and as specified below in sections 2b(2)(f), entitled Expandable Structure to Accommodate Growth and Self-adjust in Size in Vascular Segment Prostheses. Lines, and Confluence Conduits Placed in Children, as well as in section 6, entitled Description of the Preferred Embodiments of the Invention. Commands to release a drug, for example, must be complied with immediately. If, not, then sensors will signal the noncompliance to the master controller for immediate remediation using obstruction-counteractive agents.

Just referred to, section 2b(2)(f) below explains how once the patient becomes an adult so that the expansion device has expanded, a resolution of hypertension or an intermittent hypertension that would temporarily further expand the prosthesis are resolved. To avert a future need for replacement, which involves major surgery, and to assure that the pressures associated with blood flow, for example, fall within the normal ranges, expansion responsive to growth must adjust in both length and caliber in step with natural growth. To this end, internal surfaces are protected by clot and other sensors that signal the controller to release counteractant into the line through the entry or feeding side-entry device. Adhesion and accretion repellent materials are addressed in this and other sections.

The three types of blood flow switches, 1. Vascular valves and servovalves, shown in FIGS. 2, 5, 7, 8, and 10A; 2. Bloodline, or mainline, switch valves, shown in FIGS. 32A thru 32C comprising an entry jacket 169 and exit, or reentry jacket 168; and 3. Inline coupling jacket pairs, shown in FIGS. 36A thru 36G, which can be characterized as switches only during their use to transfer blood flow from a diseased or damaged segment into a tie-line prosthesis to replace the lost tissue, make possible the avoidance of interruptions in perfusion during the insertion of a bridge or prosthesis to replace a segment along a ductus such as a great vessel, along with its branches if necessary.

FIG. 36G also shows that a tie-line or bridge can serve to mount a side-entry device. This can prove beneficial when the device mounted thus cannot be positioned at the same or a closer level to the desired outlet or point of fluid delivery along the same ductus. Condensed, the actual length of the bridge depicted in FIG. 36G can be long, and the servovalve mounted, to it used as the continuously variable tapping off device feeding into a ‘tuned’ shunt as addressed below in section 2b(2)(h), entitled Local Vascular Pressure Relief, its subsections, and as shown in FIG. 22F. Tie-line mounting of flow directing components is addressed below in section 2b(2)(e), entitled Tie-line Mounted Side-entry Flow Diversion Jackets, Vascular Valves, and Servovalves.

In pediatric and adult cardiac surgery and heart transplantation, eliminated are cold storage, cardioplegia, hypothermia, cross clamping, cardiopulmonary bypass with its delayed cardiac cell death, ischemia, inflammation and traumatic incision into both the recipient and donor hearts, reperfusion injury, and general anesthesia with its postoperative cognitive impairment following cardiovascular and urological operations. Ordinary heart transplantation commonly durable for up to ten years, the degree of incisional trauma involved in a conventional heart-usually just a ventricles transplant—is very considerable. Eliminate the trauma, ischemia/reperfusion injury, general anesthesia, clamping, cardioplegia, and the durability should increase to well past ten years.

Furthermore, the elimination of these insults means that solid organ transplantation can be made available to patients ordinarily considered too debilitated, or as a neonate if not a prenate, too frail to undergo such surgery, and that outcomes should be relatively complication-free as well as more robust and durable (see, for example, Martin K R, Gamell C, Tai T Y, Bonelli R. Hansen J, and 4 others 2024. “Whole Blood Transcriptomics Reveals Granulocyte Colony-stimulating Factor as a Mediator of Cardiopulmonary Bypass-induced Systemic Inflammatory Response Syndrome,” Online, Clinical and Translational Immunology (Milton, Queensland, Australia) 13(2):e1490; Carvajal. C., Goyal, A., and Tadi, P. 2023. “Cardioplegia,” Online, Treasure Island, Florida: StatPearls Publishing Co.; Douin, D. J. Pattee. J., Scott, B., Fernandez-Bustamante, A., Prin, M., and 3 others 2023. “Hyperoxemia during Cardiac Surgery is Associated with Postoperative Pulmonary Complications,” Online, Critical Care Explorations (Philadelphia, Pennsylvania) 5(3): e0878; Ferreira, L. O., Vasconcelos, V. W., Lima, J. S., Vieira Neto, J. R., da Costa, G. E., and 10 others 2023. “Biochemical Changes in Cardiopulmonary Bypass in Cardiac Surgery: New Insights,” Online, MDPI [Multidisciplinary Digital Publishing Institute] Journal of Personalized Medicine (Basel. Switzerland) 13(10): 1506; Gilbey, T., Milne, B., de Somer, F., and Kunst, G. 2023. “Neurologic Complications after Cardiopulmonary Bypass-A Narrative Review,” Online, Perfusion (London, England) 38(8): 1545-1559; Ismail. A., Semien, G., and Miskolczi, S. Y. 2023. “Bypass Cardiopulmonary,” Online, Treasure Island, Florida: StatPearls Publishing Co.; Pollak, U., Zemmour, H., Shaked, E., Magenheim, J., Fridlich, O., and 6 others 2023. “Novel cfDNA [cell-free DNA] Methylation Biomarkers Reveal Delayed Cardiac Cell Death after Open-heart Surgery,” Journal of Cardiovascular Translational Research (New York, New York) 16(1): 199-208; Roberts, A., Duncan, E. C., Hargrave, P., Kingery, D. R., Barnes, J., Horstemeyer, D. L., and Stahl, R. F. 2023. “Complications of Cardiopulmonary Bypass from an Anesthesia Perspective: A Clinical Review,” HCA [Hospital Corporation of America] Healthcare Journal of Medicine (Nashville, Tennessee) 4(1): 13-21; Taha, A., Hjarpe, A., Martinsson, A., Nielsen, S., Barbu, M., and 4 others 2023. “Cardiopulmonary Bypass Management and Risk of New-onset Atrial Fibrillation after Cardiac Surgery,” Online, Interdisciplinary Cardiovascular and Thoracic Surgery (Oxford, England) 37(3): ivad153; Tohme, J., Lescroart. M., Guillemin, J., Orer. P., Dureau, P., and 4 others 2023. “Association between Vasoactive-inotropic Score, Morbidity and Mortality after Heart Transplantation,” Online, Interdisciplinary Cardiovascular and Thoracic Surgery (Oxford, England) 36(4): ivad055; Mayer, D., Altvater, M., Schenz, J., Arif. R., Karck, M., and 4 others 2022. “Monocyte Metabolism and Function in Patients Undergoing Cardiac Surgery,” Online, Frontiers in Cardiovascular Medicine (Lausanne, Switzerland) 9:853967; Song. B., Dang, H., Dong, R. 2021. “Analysis of Risk Factors of Low Cardiac Output Syndrome after Congenial Heart Disease Operation: What Can We Do?,” Online, BioMed Central Journal of Cardiothoracic Surgery 16(1): 135; Dilli, D., Akduman, H., Orun, U. A. Tasar, M., Tasoglu, I., and 3 others 2019. “Predictive Value of Vasoactive-inotropic Score for Mortality in Newboms Undergoing Cardiac Surgery,” Online, Indian Pediatrics (New Delhi, India) 56(6): 735-740; Merkle, J., Daka, A., Deppe, A. C., Wahlers. T., and Paunel-Gorgulu, A. 2019. “High Levels of Cell-free DNA Accurately Predict Late Acute Kidney Injury in Patients after Cardiac Surgery,” PLoS [Public Library of Science] One (San Francisco, California) 14(6): e0218548; Tidbury, N., Browning, N., Shaw, M., Morgan, M., Kemp, I., and Matata, B. 2019. “Neutrophil Gelatinase-associated Lipocalin as a Marker of Postoperative Acute Kidney Injury following Cardiac Surgery in Patients with Preoperative Kidney Impairment,” Cardiovascular and Hematological Disorders Drug Targets (Sharja, United Arab Emirates/San Francisco, California) 19(3): 239-248; Boehne. M., Sasse, M., Karch, A., Dziuba, F., Horke, A., and 4 others 2017. “Systemic Inflammatory Response Syndrome after Pediatric Congenital Heart Surgery: Incidence, Risk Factors, and Clinical Outcome,” Online, Journal of Cardiac Surgery 32(2): 116-125; Huffryer, J. L. and Groves, D. S. 2015. “Pulmonary Complications of Cardiopulmonary Bypass,” Best Practice and Research, Clinical Anesthesiology (Amsterdam, North Holland, Netherlands) 29(2): 163-175; Gaies, M. G., Gurney, J. G., Yen, A. H., Napoli, M. L., Gajarski, R. J., and 3 others 2010. “Vasoactive-inotropic Score as a Predictor of Morbidity and Mortality in Infants after Cardiopulmonary Bypass,” Pediatric Critical Care Medicine 11(2): 234-238; Hasgawa, T., Yamaguchi, M., Yoshimura, N., and Okita, Y. 2005. “The Dependence of Myocardial Damage on Age and Ischemic Time in Pediatric Cardiac Surgery,” Online, Journal of Thoracic and Cardiovascular Surgery 129(1): 192-198; Cuadrado, A. R. 2002. “Management of Postoperative Low Cardiac Output Syndrome,” Critical Care Nursing Quarterly 25(3): 63-71; Utley, J. R. 1990. “Pathophysiology of Cardiopulmonary Bypass: Current Issues,” Journal of Cardiac Surgery (Hoboken, New Jersey) 5(3): 177-189).

In conventional organ transplantation, ischemia and reperfusion injury are the most frequent causes for a complication-ridden outcome with reduced graft organ durability and increased risk of untimely rejection. Using conventional methods, a heart transplant that survives past the tenth year is exceptional. The use of regional if not local anesthesia and the avoidance of these other impediments, as well as the directly pipe-targeted delivery of medication to the sites where needed rather than indiscriminately dispersed throughout the body will reduce such problems. Another factor that will increase graft organ durability is the surveillance and the immediacy response of the medicinal and/or electrostimulatory support, and if desired, the signaling to the clinic, provided by a fully implanted prosthetic disorder response system. Such systems are addressed in U.S. Pat. No. 11,759,186, entitled Ductus Side-entry Jackets and Prosthetic Disorder Response Systems, and copending application Ser. No. 17/689,880, entitled Prosthetic Disorder Response Systems.

During cardiac operations, when a vascular valve, servovalve, bloodline switch, or inline coupling jacket pair is used to switch blood flow from a shorter native to a longer line so that the greater transit time for the blood to traverse the increase in distance would cause a momentary interruption in perfusion that might provoke a desynchronization or vasospasm, for example, a volume of blood is taken from the patient, heparinized, and injected into the longer line through the valve, switch, or inline coupling jacket accessory channel or side-stem before switching is initiated.

In U.S. Pat. No. 11,759,186, entitled Ductus Side-entry Jackets and Prosthetic Disorder Response Systems; FIGS. 16 and 21 show ductus side-entry jackets in use to allow the direct pipe-targeting of medication into ductus under the control of an implanted disorder response system that executes a prescription-program. In FIG. 16, both the mainline 13 which usually conveys blood is used to convey medication rather than blood from the injection point in body surface port 16 into the left anterior descending coronary artery, with sideline, or drugline 11 available to convey adjuvant, or accessory, medication if necessary.

This differs from the application shown in FIG. 21 of that patent wherein the mainlines 13 serve as prosthetic coronary artery bypass grafts in lieu of autologous vessels such as a right or left pedicled, mobilized, and diverted internal thoracic or internal mammary artery, or a radial, splenic, ulnar, right gastroepiploic, or inferior epigastric artery, or a less durable saphenous vein, with sideline, or drugline 11 available to convey adjuvant, or accessory, medication if necessary. Application of prosthetic bloodlines rather than vessels harvested from the body provide two major advantages: eliminating the possibility of complications and atrophy or shriveling of the tissue that had depended upon the harvested vessel as its blood supply or drainage, and providing the means to directly pipe-target medication into the lines without exposure of the medication to untargeted tissue, all the more confined with respect to the end-arterial coronaries.

Further in U.S. Pat. No. 11,759,186, druglines 11 serve as a means for medicinal and maintenance solution targeting. Avoiding the dispersion throughout the circulatory system of medicinals such as steroids, immunosuppressives, or immunosuppressants, and chemotherapeutics to treat in situ cancer, for example, and of maintenance solutions to preserve the patency of the passage through a valve, targeting avoids adverse side effects that often prevent the use of what would otherwise be the best drug for the immediate purpose. In a vascular valve, the drug and device maintenance solution-delivering line, or drugline, and the channel leading the drugline into the interior of the valve, or accessory channel, are distinguishable only from a structural standpoint.

Medicinals include anti-inflammatoires, antimicrobials, and anticoagulants, while maintenance solutions in the circulatory system include thrombolytics, and in the urinary tract, crystal solvents. Temporary use of a valve does not warrant retaining it in place for follow-up drug delivery; an independent side-entry jacket should be used instead. However, often the valving function would best be retained. Such is pertinent following a compound vascular heart transplant, where along with an implanted pacemaker/cardioverter-defibrillator, direct coordinated control over the graft organ blood supply and drainage can play a nonmedicinal part in interdicting or terminating a ventricular fibrillation, for example.

In valves such as those shown here in FIGS. 2, 5, and 8, blood preloading is injected through the accessory channel 8 or the side-stem 19; in valves such as shown in FIGS. 7 and 10A, through either accessory channel 8 or 8′, or the side-stem 19; in a bloodline switch such as shown in FIGS. 32A thru 32F through the sending valve of which the diversion chute is indicated as part number 18; and in an inline coupling-jacket, through the inlet surface of the inlet, or aortic, half of the entry jacket accessory channel, part number 8 in FIGS. 36A, 36E, 37A, 37B, and 37D.

As to terminology, the drugline is the catheteric tube that is inserted into the accessory channel of which every side-entry vascular valve, servovalve, and inline coupling jacket has at least one. The term “bloodline” refers to the synthetic, or catheteric, lines used to convey blood between native vessels. In a compound vascular bypass, or switched-organ transplant, the vessels are those of the recipient corresponding to those of the donor. In a compound vascular organ transplant, valves are used to connect corresponding vessels of the recipient to those of the donor.

Bloodline switch-valves allow transferring flow between recipient and doner suddenly, entirely, and reversibly between either of two outlet passages of which one is short and the other long. When placed in a pediatric patient, bloodlines and druglines are used that will grow with the patient. This is accomplished through the use of lines made with the expandable structure delineated below in section 2b(2)(f), entitled Expandable Structure to Accommodate Growth and Self-adjust in Size in Vascular Segment Prostheses, Lines, and Confluence Conduits Placed in Children.

When switching from longer to shorter bloodlines, the momentary increase in blood pressure due to the small increase in blood volume should be easily accommodated by the intrinsic elasticity of the shorter vessel or length of tubing used. To the same end, in a heart transplant, the vascular valves or servovalves are electronically controlled by the implanted prosthetic disorder response system to act in precise synchrony. That means for reinstating synchronization if necessary are to be immediately available is standard operating procedure.

A conventional orthotopic heart transplant usually replaces not the heart but only the ventricles and anterior halves of the atria. However, where the heart had reached end-stage failure, the impairment in function over a long period would likely have adversely affected the heart in its entirety, to include the atria. Furthermore, even setting aside the additional insults of organ preservation outside a circulatory system, clamping, cardioplegia, and the use of a cardiopulmonary bypass machine, to cut off the front of the donor and recipient hearts, then to suture the former in direct anastomotic contact with the latter despite the facts of immune function is plainly a compounding of trauma to militate against a successful outcome.

In contrast, a compound bypass heart transplant almost always removes both hearts entirely to include the atria, and peri- and postoperatively supports the replacement with constant surveillance sensors and remedial measures as necessary with the aid of a fully implanted, or ambulatory, automatic disorder response system as delineated in copending application Ser. No. 17/689,880, entitled Prosthetic Disorder Response Systems, such that the graft organ is never left unperfused. The exception is that if entirely removing the native heart would inflict needless trauma, then attaching or adhesion tissue that would not create postoperative complications were it allowed to remain may be left in place.

For the adult with end stage heart failure and the very young as well as older patients, a quicker and fundamentally less traumatic method for the transplantation of the heart, without interruption in the flow of blood or the need for general anesthesia represents a considerable reduction in trauma and the risk of complications. The elimination of the triple stage Norwood, Glenn, and Fontan operations, each posing considerable risks to provide no more than an inadequate repair of a univentricular to include a hypoplastic left heart, or a double outlet left ventricle, or tricuspid atresia, some forms of heterotaxy, or other congenital defect are all better superseded.

Combined heart and liver transplantation required as the result of defective circulation such as following Fontan repair of a univentricular heart is addressed below in section 2b(1)(b)(b7), entitled Metered Compound Vascular Bypass, or Switched-organ, Liver or Heart-Liver Transplantation. In a pediatric patient, such a series of operations only to result in an inadequate repair that will inevitably result in damage to all the organs and tissues of the body to include the brain, is so traumatizing as to lessen the odds for a favorable outcome. Alone, a conventional heart transplant is a single operation, and if the considerable trauma inflicted by this operation can be substantially reduced, the odds for success will have been materially improved. With either operation, use of the metered compound vascular bypass method critically reduces the extent of incision, the need for cardioplegia, cardiopulmonary bypass support, and general anesthesia, leaving only the entry wound as a potential source of long-term pain.

To the relative sufficiency of a conventional heart transplant, the compound vascular bypass method eliminates its major detractions which are surgical trauma to the donor and recipient organs and ischemia, along with the factors mentioned just above to include extended cardiopulmonary bypass and general anesthesia. Any solid organ can be transplanted using the compound vascular bypass method with automatic follow-up monitoring and automatic dispensing of medication to include immunosuppressive by the implanted response system which is associated with the method. This means that the zone of operable patients is materially extended to many who previously would be considered too sick to withstand the conventional surgery, and this can mean the difference between life and death.

The condition of the deceased donor heart is as important as the ability of the recipient to withstand the operation. Explantation of the graft organ by means of transecting it is to be avoided. No less important than the ability of the recipient to withstand the operation is the condition of the donor heart. Since in a compound vascular bypass transplant neither the donor nor the recipient heart is incised, here too the benefit of the compound vascular bypass approach, because to harvest the graft heart involves significantly less trauma, improves the odds for a good outcome with respect to both.

The immediate interdependence of the heart, kidneys, liver, and lungs, in fact, all the organs and tissues of the body, means that once one, if not the most, diseased major organ has been replaced and the patient allowed to heal, it is imperative that to preserve the condition of the replaced organ, as well as to further improve the health of the patient, the other organs be treated or replaced. In this, it warrants emphasizing that the fundamentally less trauma involved in the original compound vascular bypass transplant has made this further advancement toward recovery possible.

In cases where some combination of these organs have become degraded due to a lack of adequate support from one or more of the other organs, replacement along with the heart in one operation of all the affected organs, the critical reduction in the trauma obtained using the compound vascular bypass technique should considerably increase the population of patients able to undergo such a multiorgan operation where promptness makes possible an earlier and more complete recovery which can mean the survival of the patient (see, for example, Gruessner, R. W. G. and Gruessner, A. C. 2023. “Dual Living Donor Organ Transplants,” Clinical Transplantation (Copenhagen, Denmark) 37(4): e14923; Vaikunth, S., Sundaravel, S., Saef, J., and Ortega-Legaspi, J. 2023. “Novel Therapeutic Strategies in Heart Failure in Adult Congenital Heart Disease: Of Medicines and Devices,” Current Heart Failure Reports (Philadelphia, Pennsylvania) 20(5): 401-416; Fernandes, E., da Silva, F. R., Segalote, R., Rocha, P. T., Mello, F., and 14 others 2022. “Combined Heart-Liver-Kidney Transplantation: The First Experience in Latin America,” Online, Arquivos Brasileiros de Cirurgia Digestiva [Brazilian Archives of Digestive Surgery] (São Paulo, Brazil) 35: e1668; Miklin, D. J., Mendoza, M., and DePasquale, E. C. 2022. “Two is Better Than One: When to Consider Multiorgan Transplant,” Current Opinion in Organ Transplantation (Hagerstown, Maryland) 27(1): 86-91; Perez-Gutierrez, A., Siddiqi, U., Kim, G., Rangrass G, Kacha A, and 5 others 2022. “Combined Heart-Liver-Kidney Transplant: The University of Chicago Medicine Experience,” Clinical Transplantation (Copenhagen, Denmark) 36(4): e14586; Aziz, H., Kim, J., and Kwon, Y. K. 2020. “Current State of Kidney Utilization in Multiorgan Transplants,” Current Opinion in Organ Transplantation (Hagerstown, Maryland) 25(4): 316-321; Ortega-Legaspi, J. M., Hoteit, M., and Wald. J. 2020. “Immune Benefit of Combined Heart and Liver Transplantation,” Current Opinion in Organ Transplantation (Hagertown, Maryland) 25(5): 513-518; Ebong, I. A., Sayer, G., Kim, G., Jeevanandam, V., Baker, T., and 6 others 2019. “Simultaneous Heart, Liver and Kidney Transplantation: A Viable Option for Heart Failure Patients with Multiorgan Failure,” Online, Journal of Heart and Lung Transplantation (New York, New York) 38(9): 997-999; Wong, K., Tecson, K., and Cedars. A. 2019. “Outcomes of Multi-organ Transplant in Adult Patients with Congenital Heart Disease,” Online, Journal of the American Heart Association (Oxford, England) 8(22): e014088; Loebe M. 2011. “Multiple-organ Transplantation from a Single Donor,” Online, Texas Heart Institute Journal (Houston, Texas) 38(5): 555-558).

The interdependence among the organs and tissues of the body has been largely elucidated from a medical standpoint (see, for example, Berezin, A/A., Obradovic, Z., Berezina, T. A., Boxhammer, E., Lichenauer, M., and Berezin, A. E. 2023. “Cardiac Hepatopathy: New Perspectives on Old Problems through a Prism of Endogenous Metabolic Regulations by Hepatokines,” Online, MDPI [Multidisciplinary Digital Publishing Institute] Antioxidants (Basel, Switzerland) 24(6): 5089; Dutta, A., Saha, S., Bahl, J., Mittal. A., and Basak, T. 2023. “A Comprehensive Review of Acute Cardio-renal Syndrome: Need for Novel Biomarkers,” Online, Frontiers in Pharmacology (Lausanne, Switzerland) 14:1152055; Gallo, G., Lanza, O., and Savoia, C. 2023. “New Insights in Cardiorenal Syndrome: From Biomarkers to Therapy,” Online, MDPI [Multidisciplinary Digital Publishing Institute] International Journal of Molecular Sciences (Basel, Switzerland) 24(6): 5089: Kousa, O., Mullane, R., and Aboeata, A. 2023. “Cardiorenal Syndrome,” Online, Treasure Island, Florida: StatPearls Publishing Co.; Lv, J., Zhang, B., Ye. Y., Li, Z., Wang. W., and 12 others 2023. “Assessment or Cardio-renal-hepatic Function in Patients with Valvular Heart Disease: A Multi-biomarker Approach—the Cardio-renal-hepatic Score,” BioMed Central Medicine (London, England) Online, 21(1): 257; Nakashima, M., Nakamura, Nishihara, T. Ichikawa K., Nakayama, R., and 6 others 2023. “Association between Cardiovascular Disease and Liver Disease, from a Clinically Pragmatic Perspective as a Cardiologist,” MDPI [Multidisciplinary Digital Publishing Institute] Nutrients (Basel, Switzerland) 15(3): 748; Noda, T., Kamiya, K., Hamazaki, N., Nozaki, K., Ichikawa. T., and 8 others 2023. “Prognostic Impact of the Coexistence of Hepato-renal Dysfunction and Frailty in Patients with Heart Failure,” Journal of Cardiology 81(2): 215-221; Qasim, A. and Patel, J. B. 2023. “ANCA [anti-neutrophil cytoplasmic antibody]-Positive Vasculitis,” Online, Treasure, Island, Florida: StatPearls Publishing Co.; Shen, X., Guo, H., Mantica, G., and Yuan, H. 2023. “Analysis of the Clinical Features and Risk Factors of Kidney Injury in Patients with Chronic Heart Failure-A Retrospective Cohort Study,” Online, Journal of Thoracic Disease (Hong Kong, China) 15(7): 3934-3943; Stolz, L., Kirchner, M., Steffen, J., Doldi, P. M., Braun, D., and 13 others 2023. “Cardio-hepatic Syndrome in Patients Undergoing Transcatheter Aortic Valve Replacement,” Online, Clinical Research in Cardiology [in press] 19 Jun. 2023; Tasic, D., Furundzic, D., Djordjevic, K., Galovic, S., Dimitrijevic, Z., and Radenkovic, S. 2023. “Data Analysis of Impaired Renal and Cardiac Function Using a Combination of Standard Classifiers,” Online, MDPI [Multidisciplinary Digital Publishing Institute] Journal of Personalized Medicine (Basel, Switzerland) 24(6): 5089; Kieman, M. S., Udelson, J. E., and Sarnak, M. 2022. “Cardiorenal Syndrome: Definition, Prevalence, Diagnosis, and Pathophysiology,” Online at medilib.ir/uptodate/show/; Mccullough, P. A., Amin. A., Pantalone, K. M., and Ronco, C. 2022. “Cardiorenal Nexus: A Review with Focus on Combined Chronic Heart and Kidney Failure, and Insights from Recent Clinical Trials,” Online, Journal of the American Heart Association (Oxford, England) 11(11): e024139; Prastaro. M., Nardi, E., Paolillo, S., Santoro, C. Parlati, A. L. M., and 5 others 2022. “Cardiorenal Syndrome: Pathophysiology as a Key to the Therapeutic Approach in an Under-diagnosed Disease,” Online, Journal of Clinical Ultrasound (Hoboken, New Jersey) 50(8): 1110-1124; Tomita, Y., Misaka T., Yoshihisa, A., Ichijo, Y., Ishibashi, S., and 12 others 2022. “Decreases in Hepatokine Fetuin—A Levels are Associated with Hepatic Hypoperfusion and Predict Cardiac Outcomes in Patients with Heart Failure,” Clinical Research in Cardiology 111(10): 1104-1112; Di Lullo, L., Bellasi, A., Barbera, V., and Ronco, C. 2021. “Cardionephrology and Cardiorenal Disease in Italy: State of the Art,” Online, Reviews in Cardiovascular Medicine (Republic of Singapore) 22(3): 563-572; Gewehr, D. M., Giovanini, A. F., Mattar, B. A., Agulham, A. P., Bertoldi, A. S., and 3 others 2021. “Congestive Hepatopathy Secondary to Right Ventricular Hypertrophy Related to Monocrotaline-induced Pulmonary Arterial Hypertension,” Online, MDPI [Multidisciplinary Digital Publishing Institute] International Journal of Molecular Sciences (Basel, Switzerland) 22(21): 11891; Kawahira, M., Tamaki, S., Yamada, T., Watanabe. T., Morita, T., and 14 others 2021. “Prognostic Value of Impaired Hepato-renal Function and Lier Fibrosis in Patients Admitted for Acute Heart Failure,” Online, ESC [European Society of Cardiology] Heart Failure 8(2): 1274-1283; Tamarozzi, F., Fittipaldo, V. A., Orth, H. M., Richter, J., Buonfrate, D., Riccardi, N., and Gobbi, F. G. 2021. “Diagnosis and Clinical Management of Hepatosplenic Schistosomiasis: A Scoping Review of the Literature,” Online, PLoS [Public Library of Science] One Neglected Tropical Diseases (San Francisco, California) 15(3): e0009191; Kitching, A. R. Anders, H.-J., Basu, N., Brouwer, E., Gordon, J., and 7 others 2020. “ANCA [anti-neutrophil cytoplasmic antibody]-associated Vasculitis,” Online, Nature Reviews Disease Primers 6(1): 71; Okano, T., Motoki, H., Minamisawa, M., Kimura, K., Kanai. M., and 7 others 2020. “Cardio-renal and Cardio-hepatic Interactions Predict Cardiovascular Events in Elderly Patients with Heart Failure,” Online, PLoS [Public Library of Science] One (San Francisco, California) 15(10): e0241003; Raina, R., Nair, N., and Chakraborty, R. 2020. “An Update on the Pathophysiology and Treatment of Cardiorenal Syndrome,” Online, Cardiology Research 11(2): 76-88; Kumar, U., Wettersten, N., and Garimella, P. S. 2019. “Cardiorenal Syndrome: Pathophysiology”, Online, Cardiology Clinics (Amsterdam, North Holland) 37(3): 251-265; Rangaswami, J., Bhalla, V., Blair, J. E. A., Chang, T. I., Costa, S., and 2019. “Cardiorenal Syndrome: Classification, Pathophysiology, Diagnosis, and Treatment Strategies: A Scientific Statement from the American Heart Association,” Online, Circulation 139(16): e840-e878; Saladi, L, Shaikh, D., Saad, M., Candio-Rodriguez, E., D; Agait, V. D., and 3 others 2018. “Pulmonary Renal Syndrome: A Case Report of Diffuse Alveolar Hemorrhage in Association with ANCA [anti-neutrophil cytoplasmic anti body] Negative Pauci-immune Glomerulonephritis,” Medicine (Baltimore, Maryland) 97(23): e10954; Hadjiphilippou, S. and Kon, S. P. 2016. “Cardiorenal Syndrome: Review of Our Current Understanding,” Online, Journal of the Royal Society of Medicine (London, England) 109(1): 12-17; Poelzl, G. and Auer, J. 2015. “Cardiohepatic Syndrome,” Current Heart Failure Reports 12(1): 68-78; Laribi, S. and Mebazaa, A. 2014. “Cardiohepatic Syndrome: Liver Injury in Decompensated Heart Failure,” Online, Current Heart Failure Reports 11(3): 236-240).

Conditions that warrant a heart transplant include end stage decompensation due to heart failure or pulmonary fibrosis, for example, or a heart and kidney or liver transplant in cardiorenal, cardiohepatic, or the need for hepatic dialysis, or hemapheresis, for example, which no longer affords adequate organ function (see, for example, Aspromonte, N., Fumarulo, I., Petrucci, L., Biferali, B., Liguori, A., and 3 others 2023. “The Liver in Heart Failure: From Biomarkers to Clinical Risk,” Online, MDPI [Multidisciplinary Digital Publishing Institute] International Journal of Molecular Sciences (Basel, Switzerland) 24(21): 15665; Xanthopoulos, A., Starling, R. C., Kitai, T., and Triposkiadis, F. 2019. “Heart Failure and Liver Disease: Cardiohepatic Interactions,” Online, JACC [Journal of the American College of Cardiology] Heart Failure (New York, New York) 7(2): 87-97; Kobalava, Z. D., Villevalde, S. V., and Soloveva, A. E. \N 2016. “Cardio-hepatic Syndrome in Heart Failure: Prevalence, Pathogenesis, and Prognostic Significance,” [in Russian with English abstract at Pubmed] Kardiologiia (Moscow, Russia) 56(12): 63-71; additional references above in section 2b(1)(b)(b5), entitled Heterotopic Double Heart Metered Compound Vascular Bypass, or Switched-organ Transplantation [and so on]).

A reliable source of replacement neonatal hearts as addressed below in this section, in the subsection entitled Deceased Donor Sustainment Centers, likewise addressed below in this section, and orthotopic replacement using the metered compound vascular bypass method with an implanted automatic response system to monitor the status of the implant and dispense supportive medication allows doing away with the series of operations-Norwood, Glenn, Fontan- or the hybrid procedure needed to treat serious intracardiac defects which yields outcomes inferior to a transplant anyway (see, for example, Masarone, D., Kittleson, M. M., Falco, L., Martucci, M. L., Catapano, D., and 4 others 2023. “The ABC of Heart Transplantation—Part 1. Indication, Eligibility, Donor Selection, and Surgical Technique,” Online, MDPI [Multidisciplinary Digital Publishing Institute] Journal of Clinical Medicine (Basel, Switzerland) 12(16): 5217; Miller-Tate, H., Fichtner, S., Davis, J. A., Alvarado, C., Conroy, S., and 4 others 2023. “Utility of the NEONATE Score at an Institution that Routinely Performs the Hybrid Procedure for Hypoplastic Left Heat Syndrome,” Pediatric Cardiology (New York, New York) [in press August 2023]; Ferguson, M. and Shulman, M. 2022. “Cardiopulmonary Exercise Testing and Other Tests of Functional Capacity,” Online, Current Anesthesiology Reports (Philadelphia, Pennsylvania) 12(1): 26-33; Rushakoff, J. A. and Kransdorf, E. P. 2022. Heart Transplant in Older Patients,” Online, Current Transplantation Reports (Cham, Switzerland) 9(1): 48-54; Silvapulle, E., and Darvall, J. 2022. “Objective Methods for Preoperative Assessment of Functional Capacity,” Online, British Journal of Anaesthesiology Education (London, England) 22(8): 312-320; Silvapulle, E., and Darvall, J. 2022. “Subjective Methods for Preoperative Assessment of Functional Capacity,” Online, British Journal of Anesthesiology Education (London, England) 22(7): 249-257; Stolz, L., Orban, M., Beslere, C., Kresoja, K. P., Braun, D. and 15 others 2022. “Cardiohepatic Syndrome is Associated with Poor Prognosis in Patients Undergoing Tricuspid Transcatheter Edge-to-Edge Valve Repair,” Online, Journal of the American College of Cardiology. Cardiovascular Interventions (Washington, D. C.) 15(2): 179-189; Bichell, D. P. 2021. “Commentary: The Importance and Treachery of Patient Selection for Neonatal Heart Transplant,” Online, Journal of Thoracic and Cardiovascular Surgery (St. Louis, Missouri) 162(5): 1370-1371; Lin, Y., Davis, T. J., Zorilla-Vaca, A., Wojcik, B. M., Miyamoto, S. D., and 4 others 2021. “Neonatal Heart Transplant Outcomes: A Single Institutional Experience,” Journal of Thoracic and Cardiovascular Surgery (St. Louis, Missouri) 162(5): 1361-1368; ‘Hanley, C. and Wijeysundera, D. N. 2021. “Perioperative Risk Assessment-Focus on Functional Capacity,” Current Opinion in Anesthesiology 34(3): 309-316; Tanem, J., Rudd, N., Rauscher, J., Scott, A., Frommelt, M. A., and Hill, G. D. 2020. “Survival after Norwood Procedure in High-risk Patients,” Online, Annals of Thoracic Surgery (Chicago, Illinois) 109(3): 828-833; John, M. M., Razzouk, A. J., Chinnock, R. E., Bock, M. J., Kuhn, M. A., Martens, T. P., and Bailey, L. L. 2019. “Primary Transplantation for Congenital Heart Disease in the Neonatal Period: Long-term Outcomes,” Online, Annals of Thoracic Surgery (Chicago, Illinois) 108(6): 1857-1864; Sower, C. T., Romano, J. C., Yu, S., Lowery, R., Pasquali, S. K., and Zampi, J. D. 2019. “Early and Midterm Outcomes in High-risk Single-ventricle Patients: Hybrid vs Norwood Palliation,” Online, Annals of Thoracic Surgery (Chicago, Illinois) 108(6): 1849-1855; Dipchand, A. I. 2018. “Current State of Pediatric Cardiac Transplantation,” Online, Annals of Cardiothoracic Surgery (Hong Kong, China) 7(1): 31-55; John, M. and Bailey, L. L. 2018. “Neonatal Heart Transplantation,” Online, Annals of Cardiothoracic Surgery (Hong Kong, China) 7(1): 118-125; Lee, S., Kim, K. H., Hong, S. K., and Hankins, S. 2017. “Evaluation of a Heart Transplant Candidate,” [Springer Nature] Current Cardiology Reports (Philadelphia, Pennsyl 19(12): 133; Chinnock R. E. and Bailey, L. L. 2011. “Heart Transplantation for Congenital Heart Disease in the First Year of Life,” Online, Current Cardiology Reviews (Saif Zone, Sharjah United Arab Emirates/San Francisco, California) 7(2): 72-84; Chand, M., Armstrong, T., Britton, G., and Nash, G. F. 2007. “How and Why Do We Measure Surgical Risk?,” Online, Journal of the Royal Society of Medicine (London, England) 100(11): 508-512).

FIGS. 15 and 16 show a compound vascular heart transplant at the half-way point in an orthotopic, or single hear replacement, procedure, and the end-point in a heterotopic, or double heart procedure. In both, the hearts come to share a common bloodstream but are otherwise kept separate and distinct throughout, without incision, much less transection and joining along a common anastomosis which may be said to constitute the interface of ultimate direct immunological incompatibility. A heterotopic double heart transplant performed using the compound vascular bypass technique is addressed below in section 2b(1)(b)(b5), entitled Heterotopic Double Heart Metered Compound Vascular Bypass, or Switched-organ Transplantation [and so on], shown in placement of the second heart in the chest FIG. 17A in addition to FIGS. 15 and 16.

### Deceased Donor Sustainment Centers

However protective an available carrying device might be, transport of a graft organ to the operative center after having been harvested is to be avoided; any organ removed from its integral milieu in which it had developed and became physiologically stabilized will be irreversibly shocked and unavoidably degraded if removed therefrom. Instead, the prospective recipient should be transported to the nearest deceased donor sustainment center. There the prospective donor will have been placed on life-support prior to having died thus minimizing if not eliminating trauma to the prospective graft organ.

Current organ procurement measures necessarily reflect the surgical procedures employed in solid organ transplantation. Donors usually remote from prospective recipients, often a significant period of graft organ disembodiment intervenes prior to introduction of the organ into the alien and the immunologically adversarial milieu of a recipient. To mollify the shock of disembodiment and insertion into this alien and inhospitable milieu, measures to include the use of immunosuppressive and immune tolerance-inducing drugs are applied; however, following the trauma inflicted, no combination of measures could achieve much more than an inadequate palliative effect.

To withstand this ordeal, graft organs must be limited to those most robust, thus impairing while at the same time reducing the pool of available organs. An exception appears below in section 2b(1)(b)(b5), entitled Heterotopic Double Heart Metered Compound Vascular Bypass, or Switched-organ Transplantation [and so on], where an option to provide a dying patient with end stage heart failure an additional or two imperfectly functioning mutually supportive hearts is described.

While this pertains mostly to parts of the world where ventricular assist devices and artificial hearts are unavailable even for temporary use as a bridge to the placement of a sound heart once available, if practical experience of clinical trials show this approach to provide acceptable outcomes, the operation can be beneficial whenever and wherever a sound graft organ is unavailable. Another exception pertains to the use of graft organs transported under hypo- or normothermic continuous perfusion where attachment of the recipient to the transport machine or system is no different than it would have been to the graft organ while in the donor. Artificial hearts and assist devices are addressed at several additional sites in this and the sections that follow.

Death representing the ultimate trauma the tissues and organs of the body encounter, the practice of harvesting organs only once life support has been turned off and the prospective donor having agreed has died is exactly opposite to the procedure that should be followed. Many deaths occur away from a hospital, and if promptly moved to a hospital and treated, a resolute insistence on immediate proximity from the outset would only serve to reduce the pool of available graft organs.

Accordingly, if thoroughly examined and found acceptable upon arrival, graft organs transported hypo- or normothermally in continuous perfusion transport systems should be considered for transplantation (see, for example, de Aguilar Roza, B., Paim, S. M. S., Leite, R., Carbonel, A. F., Taha, M. O., and 5 others 2023. “Safe Transport of Organs and Tissues for Transplants: Technological Innovation Product Validation Method,” Online, Revista da Associação Medica Brasileira(1992), [in English] (Sao Paulo, Brazil) 69(6): e20221537; Alomari, M., Garg, P., Yazji, J. H., Wadiwala, I. J., Alamouti-Fard, E., and 3 others 2022. “Is the Organ Care System (OCS) Still the First Choice with Emerging New Strategies for Donation after Circulatory Death (DCD) in Heart Transplant?,” Online, Cureus (Palo Alto, California) 14(6): e26281; Thongprayoon, C., Kaewput, W., Pattharanitima, P., and Cheungpasitporn, W. 2022. “Progress and Recent Advances in Solid Organ Transplantation,” Online, MDPI [Multidisciplinary Digital Publishing Institute] Journal of Clinical Medicine (Basel, Switzerland) 11(8): 2112; Bezinover, D. and Saner, F. 2019. “Organ Transplantation in the Modern Era,” Online, BMC [BioMed Central] Anesthesiology(London, England) 19(1): 32; Bezinover, D. 2017. The Use of Continuous Extended Criteria Graft Perfusion Will Lead to an Increase in Transplantable Organs, Online, Transplantation (Hagerstown, Maryland) 101(7): 1525-1526).

That is, the durability of the graft will have been adversely affected, will be more likely to develop allograft vasculopathy if not cancer, adversely impacting upon its usable service life, so that a younger patient will be more likely to require a reimplantation, further reducing the pool of available organs. Using current methods of solid organ transplantation, only the best graft organs can be used and these are to an extent wasted through abuse and impaired. More recently, to better preserve their organs, donors having died in a medical center are kept sustained on life support, thus forestalling the trauma of sudden removal from their integral milieu.

However, the benefit is soon nullified when the graft organ is abruptly extracted from its host anyway. In contrast, in compound vascular bypass organ transplantation, trauma is almost entirely attributable to the need for an access wound, which is unavoidable, and in the case of a heart, lung, or heart-lung transplant this wound must be large and in itself, draining on the patient. In compound vascular organ transplantation, the blood supply and drainage vessels of the organ to be replaced are connected to those corresponding in the donor through connecting catheteric bloodlines, of which the entry into each is under the flow control of a vascular servovalve in turn under the synchronized control of a master control microprocessor. This requires that the donor and recipient be positioned side by side.

As well as to serve as a ready source of graft organs and tissues, a Deceased donor sustainment center will also provide test subjects for procedures not to be performed on a living patient. An example is provided below in section 2b(2)(j), entitled Vascular Servochokes, in the subsection thereof entitled Means for Determining the Optimal Blood Pressure for the Endothelial Uptake of Disorder-pertinent Drugs and Sufficiency of Responsive Secretory Function.

Each servovalve incorporates at least one accessory channel to admit a drugline which can drip an anticoagulant such as heparin, an immunosuppressive, anti-inflammatory, antimicrobial, or any other drug into the flow. Clot detection and eradication with platelet blockers, anticoagulants, or antithrombotics is manageable by an implanted disorder response system provided with the sensors and drugs required. This process is continuously overseen by sensors to include those used to detect rejection analyte levels, the prescription program controlling the process continuously adjusting the rate of organ transfer from the circulatory system of the donor into that of the recipient while administering the release of drugs as necessary.

Unlike conventional operations for organ transplantation, should it midprocedurally eventuate that rejection cannot be overcome, the compound vascular method is reversible so that the operator is never left without a safe means for “bailout,” Once the transfer has been completed, the valves and lines used could be removed, however, because to leave these in place allows the automatic monitoring and treatment by the implanted prosthetic disorder response system which had administered the transfer to continue, and such surveillance could make the difference in failure of success, this is discouraged. Perioperative diagnostic and therapeutic instrumentation is not limited to that encompassed in the implanted system.

As addressed in copending application Ser. No. 17/689,880, entitled Prosthetic Disorder Response Systems, the continued action of the implanted control system is able to detect the need for and effect the release of immunosuppressive and other drugs on the basis of sensor inputs. Whereas in a monomorbid system these are fed back to the system microcontroller, in a multimorbid system, feedback is to the master control microprocessor, in either case, executing its prescription-program. In this way, the system exercises a continuous or intermittent surveillance and drug administrative function which does away with the risk of prescription nonadherence.

As described herein, such a system can automatically administer a solid organ transplant or the replacement of a segment along a vessel with a prosthesis, and if necessary, can administer both during a single operation. Equally important, the same system can then provide the follow-up care needed to assure a satisfactory and durable outcome. In the case of an organ transplant, a want of diligent follow-up will result in rejection. Sensors are available to monitor all vital signs to include circulation and respiration, and the control microprocessor can control implanted resynchronization devices such as pacemaker/cardi overter-defibrillators and/or neuromodulatory electrostimulators

The substantial elimination of trauma and continued postoperative automated monitoring and treatment by the same implanted prosthetic disorder response system that administered the transplant means that replacement organs which ordinarily would not be considered suitable for transplantation may be usable, significantly increasing the pool of available replacement organs. In exigent circumstances and particularly in less developed parts of the world where a ventricular assist device, for example, is unaffordable, it is possible to replace or assist a failing organ by providing it with a backup, usually positioned heterotopically, just as the implantation of a third kidney, which is no longer thought of as extraordinary. The intense trauma inflicted on a graft organ using conventional methods necessitates that only the most robust graft organs can be used to withstand this initial insult.

Similarly, because compound vascular bypass would make solid organ transplantation fundamentally less traumatizing, and backup monitoring and immediate medicinal or synchronizing support would be provided by the fully implanted prosthetic disorder response system, two less than perfect hearts can be used where one good heart is unavailable. Improved methods of ventilation and life support has played a part in suggesting the reducing of the standards for replacement organs even when conventional transplantation is contemplated.

More recently, the literature has begun to reflect the impulse to increase the pool of organs by allowing the use of less than perfect replacement organs and—absent consideration of an implanted monitoring and automatic medicinal and resynchronization response system-includes enjoinders against such use (see, for example, Braga, V. S., Boteon, A. P. C. S., Paglione, H. B., Pecora, R. A. A., and Boteon, Y. L. 2023. “Extended Criteria Brain-dead Organ Donors: Prevalence and Impact on the Utilisation of Livers for Transplantation in Brazil,” Online, World Journal of Hepatology 15(2): 255-264; Critsinelis, A. C., Patel, S., Nordan, T., Chen, F. Y., Couper G. S., and Kawabori, M. 2023. “Trends in Outcomes of Heart Transplants Using Extended Criteria Donors: United Network for Organ Sharing Database Analysis,” Annals of Thoracic Surgery 115(6): 1503-1509; Ferrari-Light, D. and Mokadam, N. A. 2023. “Expanded Donor Pool for Heart Transplant-Yellow Flag or Open Throttle?,” Annals of Thoracic Surgery 115(6): 1509-1510; Mesnard, B., Territo, A., Campi, R., Hevia, V., Andras I., and 4 others 2023. “Kidney Transplantation from Elderly Donors (>70 Years): A Systematic Review,” World Journal of Urology 41(3): 695-707; Shin, M., Iyengar, A., Helmers, M. R., Patrick, W. L., Cohen, and 5 others 2023. “Use of Extended Criteria Donor Hearts in Combined Heart-Kidney Transplant Confers Greater Risk of Mortality,” Journal of Heart and Lung Transplantation (New York, New York) 42(7): 943-952; Weingarten, N., Iyengar, A., Herbst, D. A., Helmers, M., Meldrum, D., and 4 others 2023. “Extended Criteria Donor Organ Use for Heart-Lung Transplantation in the Modern Era,” Online, Clinics (Sao Paulo, Brazil) 78:100205; Argani, H. 2022. “Expanded Criteria Donors,” Online, Experimental and Clinical Transplantation 20(8)(Supplement 4): 13-19; Sathianathan, S. and Bhat, G. 2022. “Heart Transplant Donor Selection Guidelines: Review and Recommendations,” Current Cardiology Reports 24(2): 119-130; Yazji, J. H., Garg, P., Wadiwala, I., Alomari, M., Alamouti-Fard, E., Hussain, M. W. A., and Jacob, S. 2022. “Expanding Selection Criteria to Repairable Diseased Hearts to Meet the Demand of Shortage of Donors in Heart Transplantation,” Online, Cureus (Palo Alto, California) 14(5): e25485; Kvietkauskas, M., Leber, B., Strupas, K., Stiegler, P., and Schemmer, P. 2020. “Machine Perfusion of Extended Criteria Donor Organs: Immunological Aspects,” Online, Frontiers in Immunology (Lausanne, Switzerland) 11:192; Resch, T., Cardini, B., Oberhuber, R., Weissenbacher, A., Dumfarth, J., and 5 others 2020. “Transplanting Marginal Organs in the Era of Modern Machine Perfusion and Advanced Organ Monitoring,” Online, Frontiers in Immunology Lausanne, Switzerland) 11:631).

As addressed below in section 2b(1)(b)(b5), entitled Heterotopic Double Heart Metered Compound Vascular Bypass, or Switched-organ Transplantation [and so on], a failing but functional heart, for example, can be provided with a second heart in lieu of a good heard that might not be available or an unaffordable ventricular assist device. In marked contrast to a conventional Barnard-type heterotopic heart transplant, where—as is the case with a conventional orthotopic heart transplant—the two hearts are united in direct immunological confrontation with no ability to treat either without at the same time treating the other—the second heart remains separate and distinct from that native. Means for targeting either without at the same time releasing any of the agent or agent used into the general circulation risking adverse side effects is addressed in the patents specified above, U.S. Pat. No. 11,389,171 in particular.

Conventional orthotopic and especially heterotopic heart transplantation imposes significant trauma and often, hypoxia on the graft organ as well as demands special skill not possessed by most general surgeons. Experienced operators are found only in large centers and perform operations that last for hours while the recipient is supported on general anesthesia and a heart lung bypass machine likely to result in at least a brief period of postoperative cognitive impairment.

In comparison, compound vascular bypass heart transplantation is accomplished in much less time under regional if not local anesthesia, and is performed no differently for both orthotopic and heterotopic operations, the latter simply ended at the half-way point in the transfer of the new heart from the circulatory system of the deceased donor on life support into the circulatory system of the recipient. This simplification means that the number of available operators is expanded from a very few to include general surgeons, for example, and this in turn means that the ability to perform a heart transplant is not limited to only the largest academic medical centers where waiting times can be dangerously long, but disseminated to local hospitals.

This also means that the cost for remote surgical robotics (cybersurgery, telesurgery) and the virtually full time need for a centralized expert at a remote center are eliminated. Simplification is especially important for multiorgan transplantation in a single operation (Perez-Gutierrez, A., et al, 2022, Op cit., above in this section). Furthermore, the more complex prescription regimen and anxiety aroused by it in the patient following a multiple organ transplant is well alleviated and within the ability of the implanted prosthetic disorder response system to discharge.

Following a metered compound vascular bypass double heart heterotopic transplant, the hearts can be left ‘free-running,’ coordinated with the aid of implanted means of synchronization, or such means can be activated if and only if the need therefor becomes apparent. The intact separation between the native and graft organ and the selective treatment made possible for each can make a double transplant not just feasible but practicable.

Fundamentally less traumatizing, compound vascular bypass avoids a key adverse factor that adversely affects long-term graft organ durability. The importance of this for the patient cannot be overstated—the despair associated with the realization that the transplant organ is undergoing failure, the fear of reimplantation where organ availability may not be forthcoming, and the eventuality that the reimplant might fail are eliminated. For patient who had undergone a conventional transplant, the practicability of placing two functional organs dispels the concern that one good organ might not be available. Both eliminating the need for reimplantation with a good second graft organ and the usability of less than perfect replacement organs considerably increase the pool of available organs.

As delineated below in section 2b(1)(b)(b14), entitled Confluence Conduits: Connection of One or More Bloodlines to Flow through a Common Channel to the Same or a Different Number of Bloodlines with Self-leveling Apportionment of Outflow, means are provided to reduce the number of these connections and allow the joint application of drugs or an inline conduit pump to the flow of blood channeled through a conduit. The self-leveling feature imparted by a confluence conduit makes it possible to use a conduit with a different number of input and output bloodlines, thus simplifying transplants such as kidneys with accessory, meaning supernumerary, anomalous vessels.

While using the compound vascular method to transplant organs delivered from a distance using normothermic perfusion is possible, much of the benefit gained in situating the donor and recipient side by side is lost. For this reason, large centers should establish deceased donor sustainment centers wherein as many prospective donors as can be obtained are kept on life support past death. The measures needed to best sustain these donors involve continuous monitoring of hemodynamic, endocrine, temperature, and other functions, largely well established and addressed below in this section.

Detailed records maintained on each occupant identified as post mortem simply continue the medical record on each preceding death, with sustainment data and any scheduled donations annotated. Where the number of occupants maintained on life support is large so that the staff might not notice a sudden drop in functional capacity affecting a particular occupant, a large display similar to a flight information system along the top of each wall can include a flashing signal beside the identity of that occupant along with pertinent information.

In a conventional, or Barnard-type heterotopic heart transplant, the ventricles of the donor heart are stitched to the recipient heart with appropriate connections made to the great vessels. In a switched-organ heterotopic heart transplant, the donor and recipient hearts remain separate and distinct throughout. The advantages in being able to separately diagnosis and/or treat either heart, the direct contact of the immunologically incompatible tissues, the reversibility at any stage in the process should the need arise, and the eventual recoverability of either heart should that native recover are added to the elimination of surgical trauma, ischemia, and the risk-laden means used to support the patient during the conventional operation.

Synchronization may be unnecessary, but if it is, then medicinal and electronic means are available to effectuate such adjustments. That the hearts remain separate means not only that trauma is substantially eliminated, but that either heart can be addressed medicinally, electrically, electrophysiologically, if necessary, surgically or interventionally, and/or radiologically as distinct from the other. The trauma of the access wound in the chest is unavoidable. This extension of direct drug targetability as opposed to the dispersal of drugs throughout the circulatory system where unintended tissue is also exposed and can react negatively to either heart in a heart transplant, midprocedurally in an orthotopic and both hearts mid- and postprocedurally in a heterotopic heart transplant allows targeted treatment that can make the difference between success and failure.

Where even for a trace residue to pass into the general circulation could provoke adverse complications, several methods for confining medicinals to a target organ or segment along a ductus are provided in the antecedent applications and patents specified above. That the directly targeted delivery of drugs into the blood supply or parenchyma of either heart to the exclusion of other tissue is an ability not limited to organ transplantation but applicable to any organ or gland in the body represents a significant advancement in medical practice in itself.

However, to this ability must be added the increase in ability afforded by the implementation of a fully implanted prosthetic disorder response system. Based upon feedback received from sensors implanted at pertinent locations in the body, an implanted master control microprocessor can exercise surveillance over and control the coordinated release of drugs automatically to assure that release is timely and that the coordinated effect will be optimal in terms of overall health.

This pertains to all classes of drugs whether antiangiogenic, oncologic, anti-inflammatory, antimicrobial, neuropsychiatric, or any other class of drugs. Prosthetic disorder response systems are addressed in U.S. Pat. No. 11,759,186, entitled Ductus Side-entry Jackets and Prosthetic Disorder Response Systems, and in greater detail in copending application Ser. No. 17/689,880, of like title. Furthermore, in contrast to a conventional orthotopic or heterotopic heart transplant where neither heart has been left intact but rather sectioned and severely traumatized so that to reverse the operation has been rendered impossible, another significant implication of this intactness and separation is that regardless what or when serious complications arise, there is throughout the ability to stop and/or reverse the operation.

This in contrast to being left with no realistic ‘bailout,’ the one realistic prospect being that the patient might be sustained for a time but will certainly die. In response to this predicament, compound vascular transplantation allows either type operation to be stopped at any moment. Not only is continuation to completion not compelled, but when metered, the prosthetic disorder response control system administering the operation can be programmed to signal that the operation must be terminated and automatically undertake the measures to terminate it as safely as possible.

The foregoing and other advantages yet to be specified pertain to 1. Vascular valves and servovalves for solid organ transplantation, 2. Extracardiac repair of transposition of the great vessels, 3. Carotid endarterectomy bypass and replacement, 4 Inline coupling jackets for automatically inserting and securing a replacement vascular or ureteral segment, 5. Focused drip brachyradiochemotherapy, 6. Pressure relief of localized hypertension to include portal and pulmonary, 7. Transplantation-unassociated anastomosis bypasses, and 8. Negative feedback-controlled tuned shunts to replace “dumb” shunts such as femorofemoral and ureteroureteral, while allied devices include 9. The surgical chest dome, 10. The heart cage for heterotopic positioning of a second heart in the lower abdomen, 11. Automatic drug alternation and rotation executed by a fully implanted prosthetic disorder response system, to which must be added, 12. Automatic replacement ductus segment expansion fabric which great in tensile strength and free of seams, can replace the native vessel, usually a segment of almost all of the aorta, and expand with growth and thus spare a child the need for one or more painful revisions.

While incipient conditions which have not spread to other organs can usually be treated with a simple control system consisting of a drug reservoir, drugline, and side-entry jacket or nonjacketing side-entry connector under the control of a suitably encapsulated microcontroller, comorbid conditions generally call for a hierarchical control system in which the ground or 0 level assigns an arm of control to each morbidity, usually relating to an organ or organ system, as a component in the comorbidity.

Each higher level, or node in the hierarchy—usually a microcontroller in its own right-accepts sensor, that is, diagnostic, inputs from its subordinate nodes, determines the best joint therapy at its respective level, and passes this data up to the next higher level in the hierarchy where the therapeutic information is coordinated with that generated by an adjacent node to avoid contraindications and adverse drug-drug interactions, for example, and establish the best therapy for this subset of comorbidities.

This pattern continues up to the master control microprocessor which coordinates the sensor and prescriptive data across the sum thereof to avoid contraindications and adverse drug-drug interactions, for example, while optimizing therapy across the sum of morbidities to achieve the optimal homeostasis for the specific patient. Because congenital anomalies tend to generate comorbidities, the optimization of overall homeostasis is of fundamental significance.

### Vascular Valves and Servovalves

Vascular valves and servovalves comprise five types: those manually adjusted, shown in FIGS. 2 and 5; those highly damped nonsparking plunger solenoid-driven, shown in FIGS. 7 and 8; those servovalve-driven proportional, shown in FIG. 10A; those bloodline switches, addressed below in section 2b(1)(b)(b13), entitled Bloodline and Drugline Switch Devices: Long when Intercorporeal to Short when Intracorporeal to Accommodate Growth. shown in FIGS. 32A thru 33B; and those inline coupling jackets, shown in FIGS. 36A thru 36G and 37A thru 37C. The manually adjusted valve shown in FIG. 2 is used by the operator to set the urinary prosthesis embodiment shown in FIG. 28 while that shown in FIG. 5 is used by the patient wearer to adjust the urinary assist embodiment shown in FIG. 30.

Those manually adjusted and servovalve-driven allow switching flow between either of two outlet passages in a proportional; reversible manner, whereas those solenoid-driven such as shown in FIGS. 7 and 8 as part number 32, use highly damped, non-sparking plunger solenoids to outflow suddenly and completely between either of two outlet passages in a reversible manner. The sustainment of proper functioning of the valving devices to be described is achieved through two lines of approach. The first is the use of materials and coatings which are intrinsically clot and biofilm repellent, and the second is the direct sensor triggered pipe-targeting by an implanted disorder response system of an antimicrobial, anti-inflammatory, anticoagulant, thrombolytic, immunosuppressive, and/or crystal counteractive drip into each valving device and its substrate artery, vein, ureter, or other tubular anatomical structure. Sensors are addressed below in this section

### Comparison of Line Switched Lines to Inline Coupling Jacket Secured Lines

Line switched lines are synthetic, catheteric, extracorporeal, specifically, intercorporeal, while inline jacket secured lines are intracorporeal prostheses interposed along the length of a native vessel. The two are unrelated, not used together, and should not be confused. More specifically, whereas line switches allow switching between a number, although almost always just two, lines of different length where both are fed by and empty into vascular valve or servovalve-connected individual native vessels, inline coupling jacket-secured tie-lines are either simply cylindrical of expandable prostheses as described below in section 2b(2)(f), entitled Expandable Structure to Accommodate Growth and Self-adjust in Size in Vascular Segment Prostheses, Lines, and Confluence Conduits Placed in Children.

In either case, these are ‘anatomically’ shaped to the extent of including branches to replace a specific segment along the vessel to be replaced. The term ‘anatomical’ in this regard is not literal to include exactitude in diameter, but means that branches are included in the nonexpandable or expandable prosthesis where these are cylindrical as well. Usually used to switch between lines that differ in length, line switches could also be used to switch between lines that differ in shape, or prostheses. However, vascular prostheses are made for durability and include at least one accessory channel, part number 8 in the drawing figures, to allow the insertion of a drugline for the release of medication into the lumen. For this reason, redundancy in the form of a switchable bypass as backup between the main and the bypass is not applicable.

Moreover, a vascular prosthesis such as a replacement aortic arch with supraaortic branches affords no space to accommodate a backup to which blood flow would be switched. Accordingly, switchable bloodlines are not used to secure vascular prostheses on all three scores. In contrast, meant to replace a defective segment along a vessel and interpose a bridge line or prosthesis in its place without interruption in the flow of blood, inline coupling jackets secure the prosthesis in position at either end or as in a prosthetic aortic arch with its branches, at each outflow end, and do not need a switching capability for the same reasons that line switches do. However, referring to FIG. 37A, the defective segment to be replaced such as an abdominal aortic aneurysm must not have grown so large that the parallel relation between native and replacement segments is precluded.

In a more advanced automatic disorder response system, the release of supportive medication is not “dumb” as programmed to meet a schedule but rather directly responsive to clot, pathogen, and crystal as detected by sensors incorporated into the valves. Sensors are addressed below in this section. The direct pipe-targeting to a site of swelling of a potent anti-inflammatory such as diclofenac, meloxicam, or a steroid avoids the side effects that eventuate when these are dispersed throughout the body. Solenoid driven valves are suitable only for organ transplants under urgent circumstances. Immune tolerance-inducing agents administered by the implanted disorder response system and exogenously by medical personnel are administered prior to and during if not following the operation.

Servovalves are critically superior to solenoids in two respects—these allow a transfer of an organ from the circulatory system of the donor into that of the recipient in a controlled and graduated rather than in an abrupt manner. Upon detecting an adverse immune reaction, the system can can pause or reduce the rate of immune tolerance-inducing drug delivery. In this way, the system is able to optimize the rate of organ transfer by optimizing the rate of immune acceptance and inducing an increasing measure of microchimerization as the transfer proceeds.

Moreover, during the direct transfer of the graft organ from the circulatory system of the donor into the circulatory system of the recipient, sensors such as those specified below in this section signaling the arousal and degree of immune rejection analytes to the master control microprocessor confer the ability to automatically optimize the rate of tolerance-inducing agent delivery., While the donor and recipient will have been matched in advance, should an unexpectedly intense reaction immune or otherwise, the process of transfer can be slowed down, and if necessary, reversed to end the operation with the extent of reciprocal blood exchanged remaining, and if necessary, ameliorated postprocedurally by manually administering tolerance or other remedial medication.

Disorder response control system components are then left in the patient to be matched with another donor. If none is found, the components should remain in the recipient to allow a later attempt with a different donor and in order to allow the automatic monitoring of symptoms. The system will then administer the delivery of drugs by targeting these through the druglines situated at the system detection sites. Ordinarily, a patient in need of an organ transplant will have other organs which had been secondarily impaired and these sites are also provided with sensors and able to receive directly targeted drugs. Such a control system is generally hierarchical, wherein each impaired condition is assigned to a subsidiary control arm provided with sensors and other pertinent system components.

Executing its prescription-program, the system master control microprocessor coordinates drug delivery across the arms of control to optimize the overall homeostatic condition of the patient. That is, the fully implanted prosthetic disorder response system is enabled to continuously or intermittently monitor the function and directly pipe-target medication to the sites associated with each disease component. For example, in an organ transplant, it can make any adjustments in the apportionment of recipient/donor-blended blood through the graft organ during its transfer. By slowing down the process of organ transfer and reducing, eliminating, ie increasing the release of any drug or drugs, any stark confrontation of the immune system to the intrusion of alien tissue is minimized.

Thereafter, the programmed detection of inflammation, infection, or rejection and the targeted delivery of medication implements the continued support of the graft organ indefinitely. Such a process is fundamentally superior to any in conventional use. While still incapable of sufficient linear contraction, that is, of translating the complete distance, or throw, of an equivalent linear plunger solenoid between either of its stops, or termini, electrostatically rather than electromagnetically based, HASEL (hydraulically Amplified Self-healing ELectrostatic) and Peano-HASEL actuators will eventually reduce power consumption and pose less weight without radiating heat. Current state of the art soft robotic actuators are made by Soft Robotics, Incorporated, Bedford, Massachusetts and by Artimus Robotics, Boulder, Colorado, for example.

The capability of soft robotic actuators is currently the subject of concentrated study (see, for example, Sun, W., Zhao, B., and Zhang, F. 2023. “Design Analysis and Actuation Performance of a Push-Pull Dielectric Elastomer Actuator,” Online, MDPI [Multidisciplinary Digital Publishing Institute] Polymers (Basel, Switzerland) 15(4): 1037; Li, M., Pal, A., Aghakhani, A., Pena-Francesch, A., and Sitti, M. 2022. “Soft Actuators for Real-world Applications,” Online, Nature Reviews: Materials 7:235-249; Ma, Z. and Sameolo, D. 2022. “A Review of Electrically Driven Soft Actuators for Soft Robotics,” Online, Micromachines (Basel, Switzerland) 13(11): 1881; Rothemund, P., Kellaris, N., Mitchell, S. K., Acome, E., and Keplinger, C. 2021. “HASEL Artificial Muscles for a New Generation of Lifelike Robots-Recent Progress and Future Opportunities,” Online, Advanced Materials 33(19): e2003375; Duduta, M., Hajiesmaili, E., Zhao, H., Wood, R. J., and Clarke, D. R. 2019. “Realizing the Potential of Dielectric Elastomer Artificial Muscles,” Online, Proceedings of the National Academy of Sciences of the United States of America 116(7): 2476-2481; Kellaris, N., Gopaluni, V. V., Smith, G. M., Mitchell, S. K., and Keplinger, C. 2018. “Peano-HASEL Actuators: Muscle-mimetic, Electrohydraulic Transducers that Linearly Contract on Activation,” Science Robotics 3(14): eaar3276).

Inline coupling jackets, shown in FIG. 37A, allow the automatic removal of a diseased or malformed native segment along a native ductus, with its branches if necessary, and its immediate replacement seamlessly, without bleeding or interruption in the flow of blood. There is no conventional means for achieving such a result, and the conventional means are attended by numerous serious mid- and postoperative complications.

While primarily intended to replace congenitally defective malformed, damaged, or diseased segments along the thoracic and abdominal aortas and other major vessels to remain implanted to the end of life, if for any reason the need arises, inline coupling jackets can also replace the original replacement, and again seamlessly without interruption in the flow of blood. This requires that the original implantation has left sufficiently long segments along the stumps at either end to position the replacement inline coupling jackets.

Replacement prostheses for both pediatric and adult patients are preferably made as described below in section 2b(2)(f), entitled Expandable Structure to Accommodate Growth and Self-adjust in Size in Vascular Segment Prostheses, Lines, and Confluence Conduits Placed in Children, where placement in children is essential and placement in an adult requires that the operator manually open the structure to slightly longer than the length required. This is to allow for hypertension should it arise, the mean for returning the device to the normotensive length described in the same section.

Whereas inline coupling jackets are primarily intended for the intracorporeal replacement of blood vessels, as shown in FIG. 37B, inline coupling jackets, as can the line switches shown in FIGS. 32A thru 32E, be used to switch between vessels that differ in length. Line switches, however, are meant for intercorporeal use to exchange longer bloodlines needed to span the distance between the donor and recipient in an organ transplant for the shorter bloodlines, which can incorporate the expansion structure, which are needed for implantation.

Implanted side-entry jackets, nonjacketing side-entry connectors, vascular valves and servovalves, and inline coupling jackets all incorporate at least one accessory channel to allow the insertion therein of a drugline through which the implanted disorder response system can directly pipeline-target medication to the connector and line it connects in accordance with the system prescription-program. A synthetic segment with entry and exit bloodline switch-valves is rotated around for insertion into the native vessel transected at either end so that replacement is continuous, the flow of blood gradually transferred as to prevent bleeding, and the ends are locked in position.

A fundamental distinction in function between vascular valves, servovalves, and bloodline switches is that whereas vascular valves switch the entire flow through a vessel between that vessel and a bloodline of the reverse, and servovalves apportion flow through a single vessel between one fraction that continues through the vessel and another that is diverted into a bloodline in a continuously variable and reversible manner, a bloodline switch continues undivided flow from one bloodline into either of two bloodlines of which one is short and the other long.

Longer bloodlines often needed midprocedurally, these are changed to shorter bloodlines without interruption in the flow of blood. In pediatric patients, the shorter lines implanted must expand to accommodate growth. To this end, bloodlines made using the expandable fabric addressed below in section 2b(2)(f), entitled Expandable Structure to Accommodate Growth and Self-adjust in Size in Vascular Segment Prostheses, Lines, and Confluence Conduits Placed in Children, should allow reentry to be deferred for years.

Otherwise, two types of bloodline switches allow line switching between different length lines-bloodline switches addressed below in section 2b(1)(b)(b13), entitled Bloodline and Drugline Switch Devices: Long when Intercorporeal to Short when Intracorporeal to Accommodate Growth, and shown in FIGS. 32A thru 32C. Inline coupling jackets, addressed below in section 2b(2)(c), entitled Vascular Segment Prostheses: Inline Coupling Jackets and Bridging, or Tie-lines as shown in FIG. 37B allow the interposition of a synthetic line or tie-line prosthesis longer than the segment of the native ductus. By straddling a vascular prosthesis previously placed thus, the inline coupling jackets can insert a conduit or prosthesis of shorter length.

Considered the best option for thoracoabdominal aneurysm repair, conventional open surgery is intricate and can take an entire day with the patient under general anesthesia. Thoracoabdominal aortic aneurysms, malperfusion, and/or a rupture precipitated by chronic aortic dissection can also be approached endovascularly, with less trauma and often, in less time; however, the results are less durable (see, for example, Hong, J. C. and Coselli, J. S. 2022. “Open Repair for Thoracoabdominal Aortic Aneurysms Precipitated by Chronic Aortic Dissection,” Online, Vessel Plus 6:4; Fehrenbacher, J. W. and Corvera, J. S. 2012. “Best Surgical Option for Thoracoabdominal Aneurysm Repair—The Open Approach,” Online, Annals of Cardiothoracic Surgery (Hong Kong, China) 1(3): 334-338).

In this circumstance, an additional option for repair in the form of an optionally robotically assisted inline coupling jacket-secured prosthesis with the jackets placed automatically as delineated below in section 2b(2)(c)(c2), entitled Automatic Insertion into a Ductus of an Inline Coupling Jacket-secured Bridging, or Tie-line and Its Replacement if Necessary, the mechanism delineated below in section 6, entitled Description of the Preferred Embodiments of the Invention and depicted in FIGS. 37A thru 37E. Existing methods for the repair of a severe congenital malformity of the vascular trees in a neonate or infant include those endoluminal which significantly avoids the need to open the chest. Endoluminal repair, however, does not grow with the patient to a significant extent and often fails to hold up, instead developing leak, with the consequence that the chest must be opened anyway.

Such is an example of a half-way measure that only defers the need for more decisive action, in this case, in the form of open surgery. Instead, as asserted in copending application Ser. No. 17/689,880, entitled Prosthetic Disorder Response Systems, once an improved method which can end a life-threatening condition once and for all has been devised, it should be preferred to alternative methods incapable of expanding in step with growth and prone to failure. The convention known to be temporary from the outset, even though it requires open chest surgery, the newer method should be used. A second endoluminal repair will also be temporary, so that eventually, the direct access gained in an open operation will be needed anyway.

The woven polyethylene terephthalate polyester fiber-based prostheses used in aortic reconstruction is not inelastic, but is so to no greater an extent than will accommodate the pulse, and growth only to a limited extent. Insufficient compliance of the prosthesis and kinking results in hemolytic anemia (see, for example, Takei, Y., Shibasaki, I., Suzuki, K., Miyazaki, S., Hirota, S., and 3 others 2022. “Hemolytic Anemia Caused by An Excessively Kinked Prosthetic Graft after Total Arch Replacement Detected by 4-dimensional Flow Magnetic Resonance Imaging: A Case Report,” Online, Medicine (Baltimore, Maryland) 101(29): e29617; Tamura, K., Tatsuishi, W., Konishi, Y., Konno, N., Kato, Y., and Abe T. 2022. “Hemolytic Anemia Caused by Kinked Graft 6 Months after Aortic Dissection Repair,” Online, BioMed Central Journal of Cardiothoracic Surgery (London, England), 17(1): 308; Kitahara, H., Yoshitake, A., Hachiya, T., Okamoto, K., Kawaguchi, S., and Shimizu, H. 2016. “Kinked Graft and Anatomic Stenosis-induced Hemolytic Anemia Requiring Reoperation,” Annals of Vascular Surgery 30:308.E1-E4).

In fact, excessive expansion immediately following placement is a problem recommending some downsizing to avert regurgitation and the need for replacement of the aortic valve, and the “development of type III endoleaks in the projected landing zone” (Etz, C. D., Homann, T., Silovitz, D., Bodian, C. A., Luehr, M., and 3 others 2007. “Vascular Graft Replacement of the Ascending and Descending Aorta: Do Dacron Graft Grow?,” Online, Annals of Thoracic Surgery 84(4): 1206-1213)(see also Hori, D., Kusadokoro, S., Shimzu, T., Kimura, N., and Yamaguchi, A. 2020. “Prosthetic Graft Dilation at the Aortic Arch in the Era of Hybrid Aortic Surgery,” Online, Annals of Vascular Diseases (Tokyo, Japan) 13(2): 163-169).

Used in an adult, the woven polyester material (Dacron®) of the graft failure might not become exigent for years (see, for example, Fuladi, S., Kamerkar, D., Bhate, S., and Dhekne, A. 2019. “True Aneurysm of Dacron Graft 33 Years after Surgery,” Online, Indian Journal of Thoracic and Cardiovascular Surgery (New Delhi, India) 34(3): 383-383; Noorani, A., Ng., C., Gopalan, D., and Dunning, J. 2011. “Haemoptysis from a Dacron Graft Aneurysm 21 Years Post Repair of Coarctation of the Aorta,” Interactive Cardiovascular and Thoracic Surgery 13(1): 91-93).

Moreover, a lack of sufficient compliance with “the elastomechanical characteristic of the native arterial tissue, and the consequent lack of adequate compliance, leads to a cascade of hemodynamic and biological alterations deeply affecting cardiovascular homeostasis” (Spadaccio, C., Nappi, F., Al-Attar, N., Surtherland, F. W., Acar, C., and 4 others 2016. “Old Myths, New Concerns: The Long-term Effects of Ascending Aorta Replacement with Dacron Grafts. Not All that Glitters is Gold,” Online, Journal of Cardiovascular Translational Research (New York, New York) 9(4): 334-342) including regurgitation and a need to replace the aortic valve (see also, Pagoulatou, S., Adamopoulos, D., Rovas, G., Bikia, V., and Sterogiopulos, N. 2021. “Acute and LongOterm Effects of Aortic Compliance Decrease on Central Hemodynamics: A Modeling Analysis,” Online, Frontiers in Physiology 12:701154).

Without revision, growth would eventually cause dehiscence, rupture, and the likelihood of death. Inline coupling jacket-secured vascular prostheses made of a special expandable fabric to accommodate growth improve over both the existing operation for insertion of a prosthetic segment to replace the corresponding segment of a native vessel, and if necessary, its branches, such as the thoracic or the abdominal aorta and in the properties of the prosthesis itself in four basic respects:
1. The need to interrupt the flow of blood through the segment to be replaced, and therewith, the need for cardiopulmonary support and general anesthesia, which are the causes for numerous complications, and the possibility for ischemia-reperfusion injury are eliminated,
2. The connection of the prosthesis to the native substrate vessel up- and down-stream, and
3. The prosthesis itself are seamless, leak-free, nonmigratable, more secure, and more durable than any existing alternative, whether an elephant trunk or a frozen elephant trunk, and
4. For use in children, the tie-line, or anatomical, portion of the prosthesis secured at either end by inline coupling jackets is made with the growth-accommodating structure described below in section 2b(2)(f), entitled Expandable Structure to Accommodate Growth and Self-adjust in Size in Vascular Segment Prostheses, Lines, and Confluence Conduits Placed in Children.

The polymeric component of the fabric provides elasticity and tensile strength, and the use of inline coupling jackets along with through- and-through cross-stitching nonmigratably, nondehiscably, and seamlessly fix the prosthesis in position. Sufficient expandability to make possible placement in an infant that will last until adolescence if not later cannot be obtained from intrinsic elasticity alone which increases resistance to further stretch while expanded, and so requires that the fabric gradually unfold. Incorporating expansion vaults 234 separated by flats 233 as shown in FIGS. 15C, 15D, 36A, and 36F which forced open by the increasing pressure associated with growth, expand in step with growth.

In an infant with a congenital connective tissue defect, an initial endovascular repair of an aneurysm or coarctation along the thoracic aorta would avoid the need for open thoracic entry, and that given, no existing prosthesis would be available. For reasons specified above, the repair would almost certainly fail, and soon. Further presented with the likelihood of aneurysmal breakdown involving the supra-aortic branches, the need for open revision would likely arise after a brief interval. Here the object is to avert the need for one or more painful revisions for years if not to the end of life.

As shown in FIGS. 32A thru 32C, 33A and 33B, in conventional use, bloodline switching uses entry and exit switches, the latter resuming flow through the bloodline that preceded the entry switch. In fact, actuated by a rotary servomotor, apportionment rather than passing the entire flow through either of the two bloodlines could be accomplished, but an apportionment thus would appear to serve little if any function. Essentially, neither device is meant, nor can either perform the function of the other.

This action is irreversible; however, an original replacement segment can itself be replaced, making it reversible in this limited sense. Solenoid and servomotor-driven valves are mounted in perivascular or cinching relation to the substrate vessel, whereas the others are mounted inline upstream to the switching point. As shown in FIG. 37D, inline coupling jackets are fixed in position by being through- and-through stitched through the apertures 40 through the stumps of the native ductus outer half jackets by inserting the needle from outside the jacket through and bringing it out the opposite side, then entering from above and exiting from below, and so on.

In a child, whereas the outer half jackets expand in diameter, the inner half-jackets expand due to their expandable structure as delineated below in section 2b(2)(f), entitled Expandable Structure to Accommodate Growth and Self-adjust in Size in Vascular Segment Prostheses, Lines, and Confluence conduits Placed in Children. If felt necessary, the inner half jackets which clinch the ends of the tie-line type prosthesis can be sutured through by passing a needle heated to a temperature exceeding the melting point of the polymer through the polymeric casing. The approbation accorded transluminal procedures as less invasive notwithstanding, displacement, or migration, a serious complication encountered with inferior vena cava filters, these too would better serve if endoscopically fixed in position thus.

### Avoidance of Disruptions in Reciprocal Cross-Circulation to Include Valve-Reactive

The functions served by vascular valves and servovalves often life-sustaining, mechanical malfunctions and medical complications must be detected and counteracted promptly. In an application such as a compound vascular metered switch heart transplant, for example, the rate of straight and diverted flow moving through each vascular servovalve must at no time mid- or postoperatively be significantly altered due to changes in caliber of the substrate artery or vein due to swelling, ectasia, or stenosis, for example. For this reason, numerous integral structural features are provided to dispel the complications that would otherwise arise with the encirclement of a living ductus such as a pulsating blood artery or a peristaltic structure. Such include measures to prevent atherosclerotic degeneration of the substrate vessel due to adventitial compression and enclosure.

Accordingly, all side-entry components for perivascular positioning in relation to vessels incorporate several features to prevent unwanted changes in the internal diameter of the substrate vessel due to reactive inflammation, swelling, atherosclerotic degeneration, or late-term atrophy. These structural features include the use of nonirritating vessel adventitia-valve compliant interface investing materials to avoid compression of the adventitial vasa vasorum and nervorum and openings to give significant exposure of the adventitia to the surrounding body cavity. Such materially reduce the odds for swelling, stenosis, degeneration, or atrophy, for example, to interfere with the function of any valve as intended in the first place.

In FIGS. 2, 5, 7, 8, and 10A showing sectional views of different vascular valves, drugline 8 allows the implanted automatic disorder response system to release medication or maintenance substances into the lumen of the substrate ductus, while drugline 8′ allows the system to coat the adventitia. For valves applied to blood vessels and gamete ductus, the substances releasable thus include anticoagulants, thrombolytics, antimicrobials, anti-inflammatoires, if appropriate, immunosuppressives, and disorder-specific substances. For valves applied to ureters, crystal solvents are released through drugline 8 and those adventitia-protective through drugline 8′. Along the gastrointestinal tract, drugline 8 is used to coat the fibrosa, while digestion-related substances are released into the lumen through drugline 8′.

Also preventive is the provision of at least one direct delivery drugline to each valve which allows the automatic response system to pass an occasional drip such as a topical statin or heparin as appropriate into the valve. Reactive measures include detectors such as pressure sensing thin film sensors lining the valves to signal the automatic implanted disorder control system, which periodically monitors each valve for signs of impairment, of an interference in free flow. Addressed below in this section, sensor data indicative of obstruction due to clot or swelling signals the need to target remedial medication thrombolytic, steroidal, and/or vasodilatory directly into the affected valve or valves wherein the valve-reactive complication occurs.

Additionally, in a solid organ compound vascular bypass transplant such as of a heart, the use of a confluence conduit as addressed below in section 2b(1)(b)(b14), entitled Confluence Conduits: Connection of One or More Bloodlines to Flow through a Common Channel . . . in a direct aortocaval connection as addressed below in section 2b(1)(b)(b16), entitled Direct Aortocaval Connection: Unified Entry Arterial and Exit Venous Confluence Conduits for the Heterotopic Connection of a Graft Heart in the Lower Abdomen, reduces this problem by simply reducing the number of servovalves.

Provided it is formulated as a fluid, the drugline or druglines included in the structure of every side-entry connector, side-entry jacket, vascular valve, servovalve, tie-line, and confluence conduit can be used to deliver an intermittent drip of any medication that might be needed to support the valve itself as well as its substrate vessel. This can include nitroglycerin, a statin, or heparin, for example. For this purpose, prodrugs are prepared in their liver clearance form. Accordingly, the preservation of desired valve functioning and maintenance is secured through the combination of integral or built in features of valve structure with medicinal surveillance and maintenance by the fully implanted prosthetic disorder response system, for which this purpose-aside from many others—is justification in itself.

In especially intractable cases of valve-reactive complications, avoidance is made certain by not mounting the valve along or in contact with the vessel at all but rather along an interposed segment of a synthetic material, a type of tie-line. When the need for this is detected midoperatively, excision of a segment along the ductus and its replacement with mounted valve is accomplished without interruption in the flow of blood. Insertion of the valve premounted with the aid of inline coupling jackets at either end as shown in FIG. 36G is addressed below in section 2b(2)(c)(c2), entitled Automatic Insertion into a Ductus of an Inline Coupling Jacket-secured Bridging, or Tie-line and Its Replacement if Necessary, shown in FIGS. 37A thru 37E.

Merely to avoid contact of the valve with the substrate ductus in order to prevent a valve-reactive degenerative process, the valve is mounted along a length of tubing which little extends beyond the up- and downstream edges of the valve itself even with the length added at either end by the inline coupling jacket. As addressed below in section 2b(2)(c)(c3), entitled Types of Inline Coupling Jacket-secured Bridging, or Tie-lines, tie-line mounted servovalve, in an adult where expandability is not a consideration, the material of the mounting tube can be polyetheretherketone, stainless steel, or parylene-coated titanium, for example. Included are druglines, bloodlines, and tubular and anatomical vascular prostheses.

While components such as stent-jackets and analyte extraction jackets addressed in U.S. Pat. No. 11,389,171, entitled Integrated System for the Infixion and Retrieval of Implants, require direct contact with the substrate ductus, several different components can be mounted and fixed in position thus. Alternative connection such as shown in FIG. 31C eliminates the additional length at either end to accommodate the inline coupling jackets seen in FIG. 36G but is limited to preoperative measures where bloodless insertion is unnecessary.

In a neonate or small child, both the tie-line, or mounting tube, and the valve itself are made expandable to allow for growth without the need for revision for many years if not for life. Expandable structure for use in a neonate applies to the mounting tube, or tie-line as well as the inline coupling jackets and includes use of the expandable fabric described below in section 2b(2)(f), entitled Expandable Structure to Accommodate Growth and Self-adjust in Size in Vascular Segment Prostheses, Lines, and Confluence Conduits Placed in Children. Section 2b(2)(c)(c2), entitled Automatic Insertion into a Ductus of an Inline Coupling Jacket-secured Bridging, or Tie-line and Its Replacement if Necessary, explains how once the patient attains much growth, the tie-line if fully expanded can be replaced with one solid and adult-sized, then called a base-tube-again, without clamping and interrupting the flow of blood as would cause problems associated with ischemia.

Vascular valves and servovalves are proportional in size to the substrate ductus, usually an artery and usually small despite the impression created in the drawing figures. Greater extension of the valve-mounting tie-line up- and/or downstream allows the allows positioning the valve in any location without pinching, kinking, or abrading neighboring tissue, encroaching upon another component of the implanted disorder response system, or allowing the component toa encroach upon the vessel. As addressed below in section 2b(2)(c)(c3), entitled Types of Inline coupling jacket-secured Bridging, or Tie-lines, tie-line and solid base-tube mounted vascular valves and servovalves are used when placement must not require clamping and an interruption in blood flow with the risk of inducing vasospasm and causing ischemia.

### Application of Hierarchical Control to the Treatment of Complex Disease

Disorder response inadequacies may pertain to missing normal means, such as the congenital lack of an enzyme essential for normal digestion—an inborn error of metabolism—or may pertain to means beyond the normal, where, for example, the body is incapable of producing a suitable remedial substance or of regenerating replacement tissue other than for a bursa, a lung, or the liver, or of either healing an innate organ or generating another, such as a second, heterotopic, heart to assist that innate.

Described in U.S. Pat. No. 11,759,186, entitled Ductus Side-entry Jackets and Prosthetic Disorder Response Systems, and Ser. No. 17/689,880, entitled Prosthetic Disorder Response Systems, a prosthetic disorder response system is intended to ameliorate if not perfect any response or combination of responses that if not augmented and reinforced would otherwise leave the patient unable or inadequately able or respond to an adverse medical condition likely to worsen. More often than the result of a congenital defect, the complete lack or inadequacy of a normal response is acquired as the result of disease, injury, or surgical resection.

However, the collection of diagnostic data at the ground level means that despite organic inadequacies such as an impaired heart, liver, kidneys, lungs, or glandular function—or, the organs ultimately interdependent, any or all of these in any combination-hierarchical diagnosis and therapy is inherently tailored to the individual patient. Moreover, the continuous- or to save stored power intermittent-collection of diagnostic information means that provided prospective sites of disease have been accounted for, a change in the condition of the patient becomes immediately apparent with the therapeutic response promptly adjusted.

The term adscititious used to refer to a prosthesis or another implanted device or an organ which does not seek to emulate natural anatomy of function, whether to reinstate overall or more nearly normal health through the optimized direct pipe-targeting of drugs into blood supply of the affected organ, organs, or volume of tissue, or to employ adscititious means such as the heterotopic addition of an organ to institute more nearly normal health, the implant system requires sensors to monitor physiological and pathological indicia and motor components in the form of blood flow control valves, shunts, drug reservoir outlet control motors, and sometimes neuromodulators, for example.

Vascular valves and servovalves are meant to serve as adscititious motor end-effectors, and shunt or bypass lines are meant to serve as the ductus the control system-executing a prescription-program-uses to move and continuously adjust the volume of blood flow passed as physiological or medical conditions determine. Based upon the sum of sensor inputs, the control system in accordance with its prescription-program specifies the drugs, doses, and the activation of electromechanical and electrostimulatory devices to be used.

Applications of vascular servovalves include administering compound vascular bypass of switched solid organ transplantation, optimizing blood flow between a native and heterotopically positioned supernumerary organ enlisted to assist that native whether due to congenital anomaly, trauma, disease, or surgical resection or because it has become impaired, control the flow-through volume of a shunt used as an expedient to reduce local hypertension pending elimination of the cause such as through an organ transplant, and facilitate extracardiac correction in a transposition of the great vessels, for example.

TABLE 1
Table 1. Solenoid and servovalve flow diversion and servochoke valve-jackets.

| | Diversion Valve | | Choke Valve |
|---|---|---|---|
| | Solenoid | Servomotor | Servomotor |
| | all or nothing | controllable fraction | controllable fraction |
| Chute | yes | yes | must be manually-driven |
| | | | or servo-driven |
| Dome | no | yes, by not zero flow | yes |
| Cone | no | flow | yes |
| Driver | manual if no more | manual or linear servomotor | manual or linear |
| | than three solenoids | | servomotor |

Accordingly, the fully implanted system monitors the relevant pathophysiological indicia and automatically releases drugs or electrical stimulation, for example, as appropriate to minimize if not bring the symptoms or analytes to within the normal range. Imaging probes and other diagnostic and therapeutic end-devices under the control of an implanted microcontroller or microprocessor master controller thus execute a process of continuous negative feedback according to a prescription-program consisting of a core written primarily and generally to treat an ordinary expression of a disorder and secondarily tailored to the specific patient.

Ductus side-entry jackets and prosthetic disorder response systems were introduced in anticipation of actively controlled components in U.S. Pat. No. 11,759,186, entitled Ductus Side-entry Jackets and Prosthetic Disorder Response Systems. More specifically, described were fixed (inert, ‘dumb’) side-entry flow diversion jackets, passive components, which foreshadowed active devices such as servovalves and servochokes, adjustable in effective luminal diameter and equipped with at least one accessory channel to allow the insertion of a drugline through which any fluid drug or solution, for example, can be targeted into the device and its substrate ductus. These features are controlled in a coordinated manner by the implanted microcontroller or master control microprocessor of a prosthetic disorder response system executing a prescription-program in response to sensor feedback.

In actively capable therapeutic control systems, first generation passive, or nonadjustable, components as described in U.S. Pat. No. 11,389,171, entitled Integrated System for the Infixion and Retrieval of Implants, such as stent-jackets and impasse jackets, then became ancillary to the ‘smart’ components actively monitored and controlled by a fully implanted automatic prosthetic disorder control system. Some other passive components essential for a fully implanted prosthetic disorder response system such as sensors to signal out of normal range conditions and radiation levels, for example, are also integral components in an automatic monitoring and therapy system and are addressed herein.

Information pertaining to higher order automatic control systems in which vascular valves and servovalves may be incorporated into one or more axes of hierarchical control will be found in copending application Ser. No. 17/689,880, entitled Prosthetic Disorder Response Systems. Other active components which are the work of others include small peristaltic pumps and controllable stopcocks at the outlets to implanted drug reservoirs used to control the delivery of drugs through druglines to and through side-entry valve substrate ductus and system shunts.

Previous applications belonging to the series which include the appellation . . . and Prosthetic Disorder Response Systems' in the title comprise U.S. Pat. No. 11,759,186, entitled Ductus Side-entry Jackets and Prosthetic Disorder Response Systems and U.S. Pat. No. 11,013,858, entitled Nonjacketing Side-entry Connectors and Prosthetic Disorder Response Systems. These and U.S. Pat. No. 11,389,171, entitled Integrated System for the Infixion and Retrieval of Implants, in turn having presaged these seek to implement the treatment of serious chronic disease, life-threatening conditions, or conditions subject to recrudesce through the agency of a fully, and unless contraindicated, permanently implanted automatic disorder response system.

Such conditions include end-stage organ failure and the eradication of malignancy through transplantation or focused drip brachyradiochemotherapy, for example. A fully implanted prosthetic disorder response system includes small drug reservoirs and direct-to-target fluid drug pipelines, or druglines (drug delivery channels, druglines), and hard-wired electrical, or radio, such as Bluetooth, to allow the direct application of control commands to the ‘nidi,’ that is, the originating and emanating sites of disease.

Fully implanted prosthetic disorder response system have been described in U.S. Pat. No. 11,759,186, entitled Ductus Side-entry Jackets and Prosthetic Disorder Response Systems, as well as in copending application Ser. No. 17/689,880, entitled Prosthetic Disorder Response Systems. One reason for cross-references among these applications is that drug delivery through a line from a small drug reservoir implanted subdermally in the pectoral region is dependent upon end-junctions fastening the lines to the target structures which are stable, leak-free, and dependable. The different applications pertain to different type tissue connecting junctions any of all of which may be used in such a totally implanted automatic ambulatory disorder response system and without which such a system is not feasible.

Described here will be means for fundamental improvement in each of three types of surgical repairs based upon different types and physiological status of the tissue affected. For each of these there is eliminated the imposition, common to all conventional treatment, of a period of circulatory cessation-induced ischemia, and in the case of the heart, for example, significant surgical trauma, both of which degrade graft durability, as well as the need for general anesthesia, often leaving the patient cognitively impaired for a time. These types of tissue and their repair relate to:
1. Severely impaired major organs and glands of considerable physiological complexity for which neither surgical reconstruction nor either an organic or artificial replacement now available is capable of reinstating the organ to permanent service as a fully functioning part of the body. Ergo, an improved method for solid organ transplantation exists,
2. A great vessel, typically the thoracic and/or abdominal aorta, especially when congenitally incompatible with the normal development much less the survival of a neonate or infant. Even when baro- and chemoreceptor inclusive, such represents an example of a structure which compared to an organ such as the heart or a kidney, is relatively nonmechanical, or static.
3. The lower urinary tract as a drainage and containment system which does incorporate pressure sensing nerve endings and peristaltic function but is otherwise essentially passive and duplicable by surgical reconstruction and/or mechanically functioning prosthetics.

As to item 1, compound vascular bypass, or sudden switched-organ, or simply switch transplantation as described below applies to advanced organ failure no less in adults as in neonates and infants. The interdependency of the entire body on the proper functioning of the major organs, of which the function is highly complex, is seen, for example, in neonates born with advanced heart failure. Any significant deficiency in the heart, lungs, kidneys, liver, or autonomic system will be propagated to the rest of the body, and if left untreated, will eventually result in the death of the patient.

Due to the administration of immunosuppressives, solid organ transplantation is commonly followed by infection and malignancy. Another relatively common but unrelated sequela is an abdominal aneurysm (Englesbe, M. J., Wu, A. H., Clowes, A. W., and Zierler, R. E. 2003. “The Prevalence and Natural History of Aortic Aneurysms in Heart and Abdominal Organ Transplant Patient,” Online, Journal of Vascular Surgery (New York, New York) 37(1): 27-31).

Moreover, because the replacement organ is likely to become secondarily affected in the same as the organ that had to be replaced, the replacement of an organ at end stage disease due to a cause outside the organ itself is contraindicated. The contextual disease that secondarily affects the organ may be acquired, functional, genetic, or both. In the references, ‘Online’ means that the entire article and not just an abstract is available over the Internet. The Springer journal Frontiers of Medicine is not open-access, while Frontiers in Medicine is.

Within a few years if that, lung transplantation for pulmonary fibrosis, for example, is commonly followed by malignancy (see, for example, Kanade, R., Kler, A., and Banga, A. 2022. “Non-pulmonary Complications after Lung Transplantation: Parts 1 and 2,” Indian Journal of Thoracic and Cardiovascular Surgery 38 (Supplement 2): 280-299; Krishnan, A., Wong, G., Teixeira-Pinto, A., and Lim, W. H. 2022. “Incidence and Outcomes of Early Cancers after Kidney Transplantation,” Online, Transplant International 35:10024; Murakami, N., Webber, A. B., and Nair, V. 2022. “Transplant Onconephrology in Patients with Kidney Transplants,” Advances in Chronic Kidney Disease 29(2): 188-200; Choudhary, N. S., Saigal, S., Saraf, N., and Soin, A. S. 2021. “Extrahepatic Malignancies and Liver Transplantation: Current Status,” Online, Journal of Clinical and Experimental Hepatology 11(4): 494-500; Gogna, S., Ramakrishna, K., and Savio, J. 2021. “Post Transplantation Cancer,” Online, Treasure Island, Florida: StatPearls Publishing Co.; Hernandez-Gaytan, C. A., Rodriguez-Covarrubias, F., Castillejos-Molina, R. A., Hemancez-Porras, A., Tobia, I., Dubin, J. M. and Autran-Gomez, A. M. 2021. “Urologcal Cancers and Kidney Transplantation: A Lierature Review,” Current Urology Reports 22(12): 62; Sekulovski, M., Simonska, B., Peruhova, M., Krastev, B., Peshevska-Sekulowska, M. Spassov, L, and Velikova, T. 2021. “Factors Affecting Complications Development and Mortality after Single Lung Transplant,” Online, World Journal Transplantation 11(8): 320-334; Ruch, B., Limkermann, A. J., Garcia, P., Benedict, C., Gupta, G., Levy, M., and Sharma, A. 2020. “Tubulocystic Renal Cell Carcinoma of the Native Kidney in a Renal Tranplant Recipient: A Rare Case Report,” Online, Case Reports in Nephrology 2020:7145652; Shtraichman, O. and Ahya, V. N. 2020. “Malignancy after Lung Transplantation,” Online, Annals of Translational Medicine 8(6): 416; Yeh, C. C., Khan, A., Muo, C. H., Yang, H. R., Li, P. C., and 4 others 2020. “De Novo Malignancy after Heart, Kidney, and Liver Transplant: A Nationwide Sudy in Taiwan,” Online, Experimental and Clinical Transplantation 18(2): 224-233; Chewcharat, A. Thongprayoon, C., Bathini, T., Aeddula, N. R. Boonpheng, B. and 9 others 2019. “Incidence and Mortality of Renal Cell Carcinoma after Kidney Transplantation: A Meta-analysis,” Online, MDPI [Multidisciplinary Digital Publishing Institute] Journal of Clinical Medicine (Basel, Switzerland) 8(4): 530; Rossi, A. P. and Klein, C. L. 2019. “Posttransplant Malignancy,” Surgical Clinies of North America 99(1): 49-64; Hickman, L. A., Sawinski, D., Guzzo, T., and Locke, J. E. 2018. “Urologic Malignancies in Kidney Transplantation,” Online, American Journal of Transplantation 8(1): 13-22; Krisl, J. C., and Doan, V. P. 2017. “Chemotherapy and Transplantation: The Role of Immunosuppression in Malignancy and a Review of Antineoplastic Agents in Solid Organ Transplant Recipients,” Online, American Journal of Transplantation 17(8): 1974-1991; Gutierrez-Dalmau, A. and Campistol, J. M. 2007. “Immunosuppressive Therapy and Malignancy in Organ Transplant Recipients: A Systematic Review,” Drugs 67(8): 1167-1198; Chhajed, P. N., Bubendorf, L., Hirsch, H., Boehler, A., Weder, W. and Tamm, M. 2006. “Mesothelioma after Lung Transplantation,” Online, Thorax [British Thoracic Society/British Medical Journal] 61(10): 916-917; Picard, C., Grenet, D., Copie-Bergman, C. Martin, N., Longchampt, E., Zemoura, L., and Stem, M. 2006. “Small-cell Carcinoma of Recipient Origin after Bilateral Lung Transplantation for Cystic Fibrosis,” Journal of Heart and Lung Transplantation (New York, New York) 25(8): 981-984).

This is relevant to surgical operations monitored and postoperatively supported by an implanted automatic disorder response system because the directly pipe-targeted delivery of drugs immunosuppressive or other to specific organs, which process is automatically administered by a fully implanted prosthetic disorder response system, greatly restricts the systemic dispersal of these drugs and therefore reduces the susceptibility of the patient to infection and malignancy, such as post transplantation lymphoproliferative disease. Such means are set forth in U.S. Pat. No. 11,013,858, entitled Nonjacketing Side-entry Connectors and Prosthetic Disorder Response Systems, U.S. Pat. No. 11,389,171, entitled Integrated System for the Infixion and Retrieval of Implants; and U.S. Pat. No. 11,759,186, entitled Ductus Side-entry Jackets and Prosthetic Disorder Response Systems.

Moreover, the implanted system is able to administer, monitor, and apply adjustments as necessary in performing the transfer of the graft organ directly and seamlessly that is, with no interruption in perfusion of the graft organ, from the circulatory system of the donor into the circulatory system of the recipient. For these reasons, the life of the organ and therefore the life of the recipient should be much extended. Efforts to provide real replacement organs have yet to prove dependable over the normal life expectancy of an infant.

The abiding prospect and experience of undergoing a retransplantation every ten or so years are cardinal factors in distraction that precludes significant accomplishment, and degrades the quality of life. At least in part, the lack of durability in organ transplants as conventionally performed can be attributed to the ischemia, reperfusion injury, and severe surgical trauma inflicted on the donor, or graft organ, which is usually relegated to storage in a cold static or flowing perfusate, and may be aggravated further by the loss of innervation when the graft organ is harvested (Hofmann, J., Pühringer, M., Steinkellner, S., Holl, A. S., Meszaros, A. T., and 3 others 2022. “Novel, Innovative Models to Study Ischemia/Reperfusion-related Redox Damage in Organ Transplantation,” Online, MDPI [Multidisciplinary Digital Publishing Institute] Antioxidants (Basel, Switzerland) 12(1): 31; (Hofmann, J., Otarashvili, G., Meszaros, A., Ebner, S., Weissenbacher, A., and 7 others 2020. “Restoring Mitochondrial Function while Avoiding Redox Stress: The Key to Preventing Ischemia/Reperfusion Injury in Machine Perfused Liver Grafts?,” Online, MDPI [Multidisciplinary Digital Publishing Institute] International Journal of Molecular Sciences (Basel, Switzerland) 21(9): 3132).

The development of permanent artificial organs has been making slow progress, and has yet to demonstrate a degree of physiological sufficiency and dependability that would free the patient from onerous maintenance requirements, to include the need for constant surveillance and frequent reexamination by specialists. A fully implanted automatic prosthetic disorder response system as described in copending application Ser. No. 17/689,880, entitled Prosthetic Disorder Response Systems, would significantly reduce this burden.

And except for the insults inflicted on the graft organ when harvested, such a system would also extend the useful life of an organic replacement organ. It is the combination of an improved method for organ transplantation and continued support of a prosthetic disorder response system that will eliminate much of the trauma associated with conventional transplantation as well as provide the constant monitoring and treatment needed to exceed a conventional longevity. While the durability currently attained is adequate in the elderly, in a young patient, it remains as an abiding menace.

As to item 2, ductus segment replacement applies no less to aneurysmal defects in an adult as to congenital defects of the aorta, a more severe coarctation, and its extreme as an interrupted arch, for example, in an infant. Hardly inert plumbing, the great vessels incorporate sensors which integral to the autonomic nervous system, communicate with the hypothalamus to exert control over the circulatory and endocrine systems. Segments along a large ductus such as a vessel or the gut do not constitute distinct organs invested with complex function that eventually extends and must respond to the entire body.

Defective segments vary in extent and/or shape and lack large dedicated supply and drainage vessels. Viewed surgically, given a donor, transplantation would be relatively uncomplicated, would consist of cutting the graft to fit and anastomosing it to the host structure at either free end; however, repair through transplantation would impose a lifelong dependency upon the timely administration of immunosuppressives in addition to the prospect of eventual revision under an abiding cloud of apprehension. In fact, the implementation of prosthetic disorder response systems, described in copending application Ser. No. 17/689,880 of like title will eliminate this risk.

Thin film strain gauges and chemical sensors in such prostheses can be made to substitute for and simulate the degree of stretch-responsive to blood pressure baroreception which determines their firing rate. Innate oximetric chemoreceptors and baroreceptors are located in the carotid bodies, aortic sinus, the venae cavae, as well as other vessels such as the pulmonary vessels, and within the right side of the heart. Chemoreceptor function likewise can be simulated, and notwithstanding the exogenous, or extra-autonomic generation of the compliance obtained, can satisfy the feedback requirements of the nervous system.

The loss of these due to oncological resection or injury tends to subside in significance over time. Technically, the loss could be compensated for with prosthetic sensors and neural leads, but whether the generation of normal sensation, or experiential correlates, associated with their function would be recovered is questionable (see, for example, Zoccal, D. B., Vieira, B. N., Mendes, L. R., Evangelista, A. B., and Leirao, I. P. 2024. “Hypoxia Sensing in the Body: An Update on the Peripheral and Central Mechanisms,” Online, Experimental Physiology (Cambridge, England) 109(4): 461-469; López-Barneo, J., González-Rodríguez, P., Gao, L., Fernández-Aguera, M. C., Pardal, R., and Ortega-Saenz, P. 2016. “Oxygen Sensing by the Carotid Body: Mechanisms and Role in Adaptation to Hypoxia,” Online, American Journal of Physiology. Cell Physiology ((Bethesda, Maryland) 310(8): C629-642).

Loss of the native bodies can have an adverse therapeutic significance (see, for example, Pauza, A. G., Murphy, D., and Paton, J. F. R. 2023. “Transcriptomics of the Carotid Body,” Advances in Experimental Medicine and Biology (New York, New York) 1427:1-11; Zera, T., Moraes, D. J. A., da Silva, M. P., Fisher, J. P., and Paton, J. F. R. 2019. “The Logic of Carotid Body Connectivity to the Brain,” Online, Physiology (Bethesda, Maryland) 34(4): 264-282);

Compatible with the support of a prosthetic disorder response system, notwithstanding it represents a breach in the anatomical continuity and physiological integrity of the organism, a prosthesis nevertheless provides compensatory benefits that must, by definition, outweigh the drawbacks. A prosthesis need not be furnished with a blood supply and drainage and therefore need not be pedicled, while usually provocative of a foreign body reaction, is immunologically neutral and therefore eliminates a lifelong need for immunosuppressives. A prosthesis such as a replacement knee will often serve as a platform for the accretion of infectious bacteria but is itself oblivious to infectious and noninfectious disease and means such as the occasional application of heat may be enough to eliminate the problem.

A prosthesis is oblivious to the side effects associated with chemotherapy and radiation. Its propensity to allow the accretion of crystal in the urinary tract and thrombus in the circulatory system is usually offset readily by the application of surface treatments to dispel salt and thrombus adhesion and the intermittent automatic release of a crystal solvent or anticoagulant-usually heparin—by the implanted disorder response system, or if the patient is otherwise essentially healthy, a single dedicated line of such a system to accomplish this release. Surface treatments are addressed below in this section.

For now, rather than to transplant a segment of the gut, the affected segment is excised and the free ends, or stumps, anastomosed. While intestinal resection of fully a dozen feet of the small intestine in an adult is usually needed to induce the symptoms of malabsorption associated with the short bowel syndrome, at an adult length of around 20 to 26 feet, the removal of a smaller segment will usually have relatively little permanent effect on digestion. If malabsorption does result, the current remedy is therapy through dietary supplements or a physiologically competent graft. To replace a significant length of gut with a prosthesis would necessitate not only the ability to simulate peristalsis but reinstate the specialization of successive segments along the gut for absorbing different type nutrients, which exceed the current state of the art.

This will be accomplished over time, but for now, a transplant with automatic medicinal backup is indicated. Not synonymous with short bowel syndrome and ileus, that is, intestinal paralysis of blockage, or peristaltic dysfunction creating the effect known as pseudo obstruction, an assist device comprised of a succession of time-delayed sphincters such as shown in FIG. 10 in U.S. Pat. No. 11,759,186 entitled Ductus Side-entry Jackets and Prosthetic Disorder Response Systems, placed about the affected segment which then serves as a motor assist device to preserve the distinctive absorptive function of the paralytic native segment.

Tolerance for the excision of a segment is fundamentally different with a major vessel, which does not afford the length or an absence of branches to allow excision and direct to anastomosis. Transplantation of such a segment poses the need for a graft of the right dimensions, lifelong immunosuppressives, and in a younger patient, poses the likelihood of eventual failure and the need for revision. And while this is a problem that a prosthetic disorder response system could ameliorate if not dispel, the use of a prosthesis with automatic backup will usually prove a better way to deal with such defects than transplantation. Convention is to replace the excised segment with a polyethylene terephthalate (Dacron® E.I. DuPont de Nemours and Company Inc., Wilmington, Delaware) patch cut to shape.

In an infant, a conventional Dacron® or polytetrafluoroethylene patch aortoplasty is not a permanent repair; it does not provide a degree of expandability needed to accommodate growth and would likely deteriorate due to hydrolytic and enzymatic breakdown, necessitating periodic revisions, each posing risks and pain (Derridj, N., Ghanchi, A., Bonnet, D., Adnot, P., Rahshenas, M., and 3 others 2023. “Early Mortality in Infants Born with Neonatally Operated Congenital Heart Defects and Low or Very-low Birthweight: A Systematic Review and Meta-analysis,” Online, MDPI [Multidisciplinary Digital Publishing Institute] Journal of Cardiovascular Development and Disease (Basel, Switzerland) 10(2): 47; Mendell Blyod, V., Rinnstrom, D., Penniert, J., Ostenfeld, E., Dellborg, M., and 4 others 2022. “Interventions in Adults with Repaired Coarctation of the Aorta,” Online, Journal of the American Heart Association (Oxford, England) 11(14): e023954; Beckmann, E. and Jassar, A. S. 2016. “Coarctation Repair-redo Challenges in the Adults: What to Do?,” Online, Journal of Visualized Surgery (Hong Kong, China) 4:76; Cramer, J. W., Ginde, S., Bartz, P. J., Tweddell, J. S., Litwin, S. B., and Earing, M. G. 2013. “Aortic Aneurysms Remain a Significant Source of Morbidity and Mortality after Use of Dacron® Patch Aortoplasty to Repair Coarctation of the Aorta: Results from a Single Center,” Pediatric Cardiology 34(2): 296-301; Karamlou T., Bernasconi, A., Jaeggi, E., Alhabshan, F., Williams, W. G., and 3 others 2009. “Factors Associated with Arch Reintervention and Growth of the Aortic Arch after Coarctation Repair in Neonates Weighing Less than 2.5 kg,” Online, Journal of Thoracic and Cardiovascular Surgery 137(5): 1163-1167; Mendelsohn, A. M., Crowley, D. C., Lindauer, A., and Beekman, R. H. 3d 1992. “Rapid Progression of Aortic Aneurysms after Patch Aortoplasty Repair of Coarctation of the Aorta,” Journal of the American College of Cardiology 20(2): 381-385; Sade, R. M., Crawford, F. A., Hohn, A. R., Riopel, D. A., and Taylor, A. B. 1984. “Growth of the Aorta after Prosthetic Patch Aortoplasty for Coarctation in Infants,” Annals of Thoracic Surgery 38(1): 21-25). Here the object is to make the replacement segment of a material and configuration that will spontaneously expand with growth until adulthood without the need for revision.

While a coarctation can sometimes be ameliorated by excision or transluminal balloon expansion and stenting or endovascular repair, which latter is not likely to remain intact to the end of life, such is not applicable to all but the smallest aneurysm, for example. For an adequate repair, the junctions with the native vessel at either end must also comply with the expansion in growth and must never leak. To be avoided is the need to repeat, much less multiple times, a midsternal thoracotomy or hemi-clamshell and therewith the pain associated with a thoracic entry wound. The seamless repair completely replaces the affected vessel, leaving no area to the sides to fail later.

Prostheses to replace a segment along the aorta which will expand in step with growth are described below beginning with section 2b(2)(c), entitled Vascular Segment Prostheses: Inline Coupling Jackets and Bridging, or Tie-lines. While repair using autologous tissue can elude a failure of the patch to grow with the patient, the placement of these is often plagued by restenosis and requires cross-clamping and a period of anoxia lasting about a half hour (see, for example, Allen, B. S., Haldorsson, A. O, Barth, M. J., and Ibawi, M. N. 2000. “Modification of the Subclavian Patch Aortoplasty for Repair of Aortic Coarctation in Neonates and Infants,” Online, Annals of Thoracic Surgery 69(3): 877-880).

More recent and improved techniques using autologous tissue should afford expansion with growth, but albeit less frequently, require a period of anoxia, and subject to restenosis (see, for example, Li, C., Ma, J., Yan, Y., Chen, H., Shi, G., Chen, H., and Zhu, Z. 2022. “Surgical Options for Proximal and Distal Transverse Arch Hypoplasia in Infants with Coarctation,” Online, Translational Pediatrics (Hong Kong, China) 11(3): 330-339; Kozyrev, I. A., Kotin, N. A., Averkin, I. I., Ivanov, A. A., Latypov, A. A., and 4 others 2021. “Modified Technique for Coarctation of Aorta with Hypoplastic Distal Aortic Arch,” Journal of Cardiac Surgery 36(6): 2063-2069; Chen, W., Fan, C, Tang, S., Zhou, W., Iroegbu, C. D., Li, J., Wu, X., and Yang, J. 2020. “Application of Modified Sliding Anastomosis in the Repair of Aortic Coarctation,” Online, BioMed Research International 2020:3805385; Dharmapuram, A. K., Ramadoss, N., Verma, S., Vejendla, G., an Ivatury, R. M. 2018. “Early Outcomes of Modification of End to Side Repair of Coarctation of Aorta with Arch Hypoplasia in Neonates and Infants,” Online, Annals of Pediatric Cardiology 11(3): 267-274).

### Interdiction of Metastasis Through Transplantation

While barring the development of safe and effective gene therapy or organ regeneration through the organ germ method (references below), to replace an organ such as an ovary, into a hostile milieu that would affect the graft organ no differently than it had that native, such as pathogenic variants of the BRCA1/2 genes, is futile (see, for example, Shah, S., Cheung, A., Kutka, M., Sheriff, M., and Boussios, S. 2022. “Epithelial Ovarian Cancer: Providing Evidence of Predisposition Genes,” Online, MDPI [Multidisciplinary Digital Publishing Institute] International Journal of Environmental Research and Public Health (Basel, Switzerland) 19(13): 8113).

Absent pathogenic variants of the BRCA1/2 genes, and cancer-free, the patient would not have been prompted to have ova preserved for later use through in vitro fertilization. Left untreated, bilateral nongenetically premetastatic ovaries would later spread malignancy to the rest of the body, and as would polycystic ovarian syndrome, adversely affect endocrine function (see, for example, Sangaraju, S. L., Yepez, D., Grandes, X. A., Talanki Manjunatha, R., and Habib, S. 2022. “Cardio-metabolic Disease and Polycystic Ovarian Syndrome (PCOS): A Narrative Review,” Online, Cureus (Palo Alto, California) 14(5): e25076; Osibogun, O., Ogunmoroti, O., and Michos, E. D. 2020. “Polycystic Ovary Syndrome and Cardiometabolic Risk: Opportunities for Cardiovascular Disease Prevention,” Online, Trends in Cardiovascular Medicine 30(7): 399-404).

Ovaries not genetically predisposed to malignancy are specified because otherwise disease-free replacement of either or both ovaries would likely be no less susceptible to malignancy. A heart impaired for any reason, whether due to a congenital defect in a neonate or congestive heart failure in an adult, creates its own hostile milieu; that is, the entire body dependent upon the normal functioning of the endocrine system and the heart, the longer an inadequacy of oxygenation is allowed to continue, the more defective will the other organs upon which the heart depends in return will progress.

Cardiopulmonary, cardiohepatic, and cardiorenal syndromes all attest to this fact. For this reason, a malfunction of the heart and of any other organ or a defect such as an inborn error in metabolism or endocrine function is best interdicted before a state of degradation sets in such that replacement with a good heart alone will no longer be able to return the patient to a state of overall good health. But it can still save his life.

However, where an organ, or if paired, the pair bilaterally affected by premetastatic malignancy, common with the ovaries, but here as result not of a genetic predisposition but rather a random cause such as exposure to radiation, the fertility drug clomiphene citrate, progesterone, luteinizing hormone, follicle-stimulating hormone, or gonadotropins, remains confined to those organs before having metastasized elsewhere, the disease can be eradicated through transplantation.

Thalidomide prescribed for inflammatory bowel disease can diminish ovarian reserve beyond what inflammatory bowel disease itself might inflict (see, for example, Guo, L., Liu, Y., Li, J., Liu, Q., Liu, B., and Shi, X 2023. “Inflammatory Bowel Disease Can Reduce Ovarian Reseerve Function in Women: A Systematic Review and Meta-analysis,” Online, Medicine (Baltimore, Maryland) 102(11): e33113; Fujimori, M., Takahashi, T., Furukawa, Y., Takanashi, A., Lizawa, Y., and 4 others 2022. “Synchronous Bilateral Primary Ovarian Cancer with Right Endometroid Carcinoma and Left High-grade Serous Carcinoma: A Case Report and Literature Review,” Online, BioMed Central Women's Health 22(1): 103; Mukuda, N., Fujii, S., Inoue, C., Fukunaga, T., Oishi, T., Harada, T., and Ogawa, T. 2018. “Bilateral Ovarian Tumors on MRI: How Should We Differentiate the Lesions?,” (in English), Online, Yonago Acta Medica (Yonago, Japan) 6(12): 110-116; Peng, X., Zhi, M., Wei, M., Li, T. T., Zhang, M., and 9 others 2017. “Thalidomide Results in Diminished Ovarian Reserve in Reproductive Age Female IBD [inflammatory bowel disease] Patients,” Online, Medicine (Baltimore, Maryland) 96(21): e6540).

In such an eventuality, a better resolution can be offered. The resolution consists first of preserving unaffected ova, and secondly, of having the affected ovaries replaced before the malignancy spreads. While the first component is conventional, the latter recognizes that the ovaries are essential for preserving normal hormonal function. Allogeneic ovaries taken from a younger patient maintained on life support past expiration produce genetically distinct ova but it is the ova obtained from the native ovaries that are to be used, while the healthy replacement ovaries can provide a more integral and natural form of hormone replacement therapy for the recipient to carry her own fetus and/or sustain consistent function once postmenopausal.

Thalidomide also has some chemotherapeutic value in the treatment of ovarian cancer, but not without serious side effects that might not dissipate once taking the drug has stopped (see, for example, CancerConnect 2018. “Thalidomide for Ovarian Cancer Has Benefits but Doesn't Improve Overall Survival,” Online, cancerconnect.com; Tempfer, C. B., Schutheis, B., Hilal, Z., Dogan, A., and Rezniczek, G. A. 2017. “Thalidomide and Lenalidomide for Recurrent Ovarian Cancer: A Systematic Review of the Literature,” Online, Oncology Letters (Athens, Greece) 14(3): 3327-3336).

For minimal complications, the transplant, preferably orthotopic, uses the metered compound vascular bypass technique administered by the prosthetic disorder response system. Transplantation of the pair is not simultaneous but sequential, anomalies aside, using the ovarian branches of the abdominal aorta and the ovarian veins of which that on the right drains directly into the inferior vena cava while that on the left drains into the left renal vein.

Medicinally supported by an implanted disorder response system which dispenses immunosuppressive and collateral medication on the basis of intermittent sensor inputs automatically, intermittent diagnostics and drug administration during gestation and thereafter for immunosuppression is reduced to occasional replenishment of small drug reservoirs ordinarily implanted in the pectoral region such as shown in FIGS. 26A and 26B, using simple tools such as shown in FIGS. 27A, and 27B.

For compound vascular bypass organ transplantation, the graft organ is directly transferred from the circulatory system of the donor living or sustained on life support into the circulatory system of the recipient without interruption in blood flow making ischemia and reperfusion injury and the immune reaction thereto irrelevant. The ovaries transferred directly from the donor into the recipient without interruption in blood flow, cold storage, or ischemia-reperfusion injury, for example, should provide a superior outcome (see, for example, Chen, Z., Liu, Y., Lin, Z., and Huang, W. “cGAS-STING [cyclic guanosine monophosphate-adenosine monophosphate (cGAMP) synthase-stimulator of interferon (IFN) genes (cGAS-STING) pathway] Pathway in Ischemia-Reperfusion Injury: A Potential Target to Improve Transplantation Outcomes,” Online, Frontiers in Immunology (Lausanne, Switzerland) 14:1231057; Tang, Q., Dong, C., and Sun, Q. 2022. “Immune Response Associated with Ischemia and Reperfusion Injury during Organ Transplantation,” Inflammation Research (Basel, Switzerland) 71(12): 1463-1476; Sheshpari, S., Shahnazi, M., Mobarak, H., Ahmadian, S., Bedate, A. M., and 4 others 2019. “Ovarian Function and Reproductive Outcome after Ovarian Tissue Transplantation: A Systematic Review,” Online, BioMed Central Journal of Translational Medicine 17(1): 396). In this way, an improved method of organ transplantation, along with the support of an implanted response system to monitor and automatically and directly pipe-target medication directly to the graft ovaries, can approximate a cure.

Provided the patient is otherwise gestationally competent, with a normal uterus and free of serious cardiac and/or renal disease, the recovery of ovarian function through an improved method of transplantation should materially improve the durability and sufficiency of the outcome. In many cases, this process will eliminate the need to use a gestational carrier, or surrogate with its potential complications and expense, as well as reduce the need for exogenous medication to include hormonal during gestation.

The organ germ method is a form of tissue engineering which for the time being, remains experimental (Ikeda, E., Ogawa, M., Takeo, M., and Tsuji, T. 2019. “Functional Ectodermal Organ Regeneration as the Next Generation of Organ Replacement Therapy,” Online, Royal Society Open Biology 9(3): 190019; Ogawa, M. and Tsuji, T. 2017. “Functional Salivary Gland Regeneration,” Methods in Molecular Biology. Organ Regeneration 1597:135-151; Ogawa, M. and Tsuji, T. 2015. “Functional Salivary Gland Regeneration as the Next Generation of Organ Replacement Regenerative Therapy,” Odontology 103(3): 248-257; Ogawa, M., Oshima, M., Imamura, A., Sekine, Y., Ishida, K., and 5 others 2013. “Functional Salivary Gland Regeneration by Transplantation of a Bioengineered Organ Germ,” Online, Nature Communications (London, England) 4:2498).

### Surgical Treatment of a Congenitally Malformed Heart and Aorta in a Prenate or Neonate

Unsurvivable, a congenitally hypoplastic left ventricle, univentricular malformities, and other severe congenital malformities such as tetralogy or pentalogy of Fallot demand therapeutic intervention, the sooner the more severe is the malformity. Lesser malformities can be survivable but significantly impair development. Conventionally a univentricular heart is dealt with through three successive and intricate intracardiac operations—the Norwood, Glenn, and Fontan—after which the patient usually dies within 5 years, and if not, remains quite sick and exhibits cognitive impairment. At the completion of the Fontan procedure, the patient is left with pulseless, or Fontan′ circulation (see John, M. M. and Mckenzie, E. D. 2020. “Norwood Procedure: How I Do It,” Journal of Thoracic and Cardiovascular Surgery Techniques (New York, New York) 4:205-207; Redmond, J. M. 2019. “Bidirectional Glenn and Hemi-Fontan Procedures,” in Spray, T. L. and Acker, M. A. (eds.), Operative Cardiac Surgery, Didcot, Oxfordshire, England: Taylor & Francis; Fontan, F. and Baudet, E. 1971. “Surgical Repair of Tricuspid Atresia,” Thorax (London, England) 26(3): 240-488), which much like the circulation obtained with a ventricular assist device, is abnormal and leads to adverse results. The need for a heart of heart-liver transplant secondary to sustained Fontan circulation is addressed below in section 2b(1)(b)(b7), entitled Metered Compound Vascular Bypass, or Switched-organ Liver or Heart-Liver Transplantation.

A heart severely malformed at birth is not capable of repair that would provide normal circulation, much less over a normal lifetime. The surgical approach prevents death shortly after birth but leads to a low quality, short life with much apprehension and grief for the parents. The preceding section, entitled Interdiction of Metastasis through Transplantation, emphasized that because the rest of the body is dependent upon the heart, whether the heart is defective due to a congenital malformity in an infant or due to heart failure in an adult, the longer amelioration if not correction is postponed, the more time will the other organs and tissues deteriorate.

Congenital defects are often multiple, so that if the lungs, liver, and/or kidneys are defective, these too must be replaced, or if an inborn error in metabolism that militates against the heart is present, this must be counteracted. In less severe malformities, the adequacy of surgical repair is often satisfactory. All such therapy is the more effective the sooner it is accomplished. Reference pertaining to the complications that often follow surgical reconstruction of the univentricular heart are provided below in section 2a(1), entitled Congenital Defects of the Heart and Other Organs—the Importance of Prompt Intervention

However, without surgical intervention, tetralogy of Fallot and similar malformities are seldom survivable beyond 20 sick years of age (see, for example, Tsui, C., Wan, D., Grewal, J., Kiess, M., Barlow, A., Human, D., and Chakrabarti, S. 2021. “Increasing Age and Atrial Arrhythmias are Associated with Increased Thromboembolic Events in a Young Cohort of Adults with Repaired Tetralogy of Fallot,” Online, Journal of Arrhythmia (Tokyo, Japan) 37(6): 1546-1554; Dennis, M., Moore, B., Kotchetkova, I, Pressley, L., Cordina, R., and Celermajer, D. S. 2017. “Adults with Repaired Tetralogy: Low Mortality but High Morbidity up to Middle Age,” Online, British Medical Journal Open Heart 4(1): e000564; Gorla, R., Macchi, A., Franzoni, I., Rosa, I., Buzzetti, F., Pavon, A. G., and Margonato, A. 2012. “Unrepaired Tetralogy of Fallot in an 85-year-old Man,” Congenital Heart Disease 7(5): E78-E81). Adequacy of repair comprehends the elimination of symptoms, which is seldom if ever the case.

Myocardial fibrosis is another postoperative eventuality (see, for example, Dobson, R. J., Mordi, I., Danton, M. H., Walker, N. L., Walker, H. A., and Tzemos, N. 2017. “Late Gadolinium Enhancement and Adverse Outcomes in a Contemporary Cohort of Adult Survivors of Tetralogy of Fallot, Congenital Heart Disease 12(1): 58-66). Also among the risks faced by patients following surgical repair is atrial or ventricular tachyarrhythmia and sudden cardiac death can result from right atrial area and right ventricular outflow tract akinetic length (Bonello, B., Kempny, A., Uebing, A., Li, W., Kilner, P. J., and 6 others 2013. “Right Atrial Area and Right Ventricular Outflow Tract Akinetic Length Predict Sustained Tachyarrhythmia in Repaired Tetralogy of Fallot,” Online, International Journal of Cardiology (Amsterdam, North Holland, Netherlands) 168(4): 3280-3286).

While able to provide acceptable results that allow a heart transplant using conventional technology with the life-long need to remember to take the immunosuppressive medication to be avoided in the majority of patients, after repair of tetralogy of Fallot, impairment of blood flow to the lungs and/or a leaking pulmonary valve may cause dyspnea and regurgitation requiring valve replacement. A repaired tetralogy of Fallot heart that requires more than one post-repair operation should be considered for replacement. An improved method of solid organ transplantation and implanted automatic disorder response system for follow-up monitoring and treatment as necessary would eliminate the greater risk associated with repair.

Post-repair symptoms of pulmonary regurgitation may include fatigue, chest pain, edema in the lower extremities, cyanosis, dizziness, syncope, and arrhythmias (see, for example, Huang, Y., Cai, X., Zhong, L., Xie, W., Lou, Q., and 5 others 2023. “End-diastolic Forward Flow in Repaired Tetralogy of Fallot: Mid-term Outcomes from a Single Center,” Online, Frontiers in Cardiovascular Medicine (Lausanne, Switzerland) 9:1068752; Arya, N., Schievano, S., Caputo, M., Taylor, A. M., and Biglino, G. 2022. “Relationship between Pulmonary Regurgitation and Ventriculo-Arterial Interactions in Patients with Post-early Repair of Tetralogy of Fallow: Insights from Wave-intensity Analysis,” Online, MDPI [Multidisciplinary Digital Publishing Institute] Journal of Clinical Medicine (Basel, Switzerland) 11(20): 6186; van den Eynde, J., Derdeyn, E., Schuermans, A., Shivaram, P., Budts, W., Danford, D. A., and Kutty, S. 2022. “End-diastolic Forward Flow and Restrictive Physiology in Repaired Tetralogy of Fallot: A Systematic Review and Meta-analysis,” Online, Journal of the American Heart Association (Oxford, England) 11(7): e024036; Faerber, J. A., Huang, J., Zhang, X., Song, L., DeCost, G., and 7 others 2021. “Identifying Risk Factors for Complicated Post-operative Course in Tetralogy of Fallot Using a Machine Learning Approach,” Online, Frontiers in Cardiovascular Medicine (Lausanne, Switzerland) 8:685855; van den Bosch, E., Bogers, A. J. J. C., Roos-Hesselink, J. W., van Dijk, A. P. J., van Wijngaarden, M. H. E. J., and 6 others 2020. “Long-term Follow-up after Transatrial-Transpulmonary Repair of Tetralogy of Fallot: Influence of Timing on Outcome,” Online, European Journal of Cardiothoracic Surgery (Berlin, Germany) 57(4): 635-643; Dluzniewska, N., Podolec, P., Skubera, M., Smas-Suska, M., Pajak, J., and 4 others 2018. “Long-term Follow-up in Adults after Tetralogy of Fallot Repair,” BioMed Central Cardiovascular Ultrasound 16(1): 28).

“Every repaired Fallot is inevitably a damaged heart. Consequently, repair of this condition cannot be curative at present; it is palliative surgery,” (Hosseinpour, A. R., Gonzalez-Calle, A., Adsuar-Gomez, A., and Ho, S. Y. 2021. “The Predicament of Surgical Correction of Tetralogy of Fallot,” Pediatric Cardiology (New York, New York) 42(6): 1252-1257). Surgical repair notwithstanding, postoperative deformation of the ventricles, histopathology of the outflow tract, and valve problems can require follow-up operations for years (see, for example, Castellanos, D. A., Skardova, K., Bhattaru, A., Berberoglu, E., Greil, G., and 6 others 2021. “Left Ventricular Torsion Obtained Using Equilibrated Warping in Patients with Repaired Tetralogy of Fallot,” Online, Pediatric Cardiology (New York, New York) 42(6): 1275-1283; erratum, Pediatric Cardiology 2022, 43(1): 248; Mauger, C. A., Govil, S., Chabiniok, R., Gilbert, K., Hehde, S., and 9 others 2021. “Right-Left Ventricular Shape Variations in Tetralogy of Fallot: Associations with Pulmonary Regurgitation,” Online, BioMed Central Journal of Cardiovascular Magnetic Resonance 23(1): 105; Li, V. W., Yu, C. K., So, E. K., Wong, W. H., and Cheung, Y. F. 2020. “Ventricular Myocardial Deformation Imaging of Patients with Repaired Tetralogy of Fallot,” Journal of the American Society of Echocardiography 33(7): 788-801).

The aorta and outflow tract in tetralogy of Fallot are often defective, but since in compound vascular orthotopic heart transplantation the heart is removed from the donor intact without having been incised, the great vessels are harvested attached to the heart proper. In a conventional heart transplant, the ventricles rather than the intact heart are harvested, which would make transplantation of the great vessels a second operation (see, for example, Zhang, S., Liu, H., Wang, X., Huang, S., and Zhang, C. 2022. “The Surgical Strategy for Progressive Dilatation of Aortic Root and Aortic Regurgitation after Repaired Tetralogy of Fallot: A Case Report,” Online, Frontiers in Cardiovascular Medicine 9:840946; Chowdhury, U. K., Sankhyan, L. K., Avneesh, S., Ray, R., Kalaivani, M., Hasja, S., and Chauhan, A. 2020. “Histologic Abnormalities of the Ascending Aorta: Effects on Aortic Remodeling after Intracardiac Repair of Tetralogy of Fallot,” Online, Texas Heart Institute Journal 47(2): 86-95; Schafer, M., Barker, A. J., Morgan, G. J., Jaggers, J., Stone, M. L, and 3 others 2020. “Increased Systolic Vorticity in the Left Ventricular Outflow Tract is Associated with Abnormal Aortic Flow Formations in Tetralogy of Fallot,” International Journal of Cardiovascular Imaging 36(4): 691-700; Ramaprabhu, K., Idhrees, M., and Velayudhan, B. 2019. “Aortopathy in Tetralogy of Fallot-A Collective Review,” Online, Indian Journal of Thoracic and Cardiovascular Surgery (New Delhi, India) 35(4): 575-578; Chowdhury, U. K., Avneesh, S., Ray, R., Reddy, S. M., Kalaivani, M., Hasija, S., and Kumari, L. A. 2018. “A Comparative Study of Histopathological Changes in the Ascending Aorta and the Risk Factors Related To Histopathological Conditions and Aortic Dilatation in Patients with Tetralogy of Fallot and a Functionally Univentricular Heart,” Heart Lung and Circulation 27(8): P1004-P1010; Schafer, M., Browne, L. P., Morgan, G. J., Barker, A. J., Fonseca, B., Ivy, D. D., and Mitchell, M. B. 2018. “Reduced Proximal Aortic Compliance and Elevated Wall Shear Stress after Early Repair of Tetralogy of Fallot,” Online, Journal of Thoracic and Cardiovascular Surgery 156(6): 2239-2249).

In the repair of a congenitally hypoplastic left ventricle, the initial operation alone, the Norwood, can last eight hours with the baby on cardiopulmonary bypass life support, making cognitive deficiency more likely, as well as a month in the hospital (see, for example, Urencio, M., Greenleaf, C., Salazar, J. D., and Dodge-Khatami, A. 22016. “Resource and Cost Considerations in Treating Hypoplastic Left Heart Syndrome,” Online, Pediatric Health, Medicine and Therapeutics (Auckland, New Zealand) 7:149-153).

The object in medical treatment should be not a short survival without, hope but rather once and for all, to eradicate the morbidity in a single decisive and materially less traumatic operation. While to replace the heart without opening the chest is impossible, to do so without opening the heart is possible. The replacement of such a heart and defective aorta through the compound vascular bypass technique is accomplished in a single operation during which the donor heart itself is never ischemic-essentially continues to beat and remains intact throughout the operation. The combination of a reduction in trauma and the elimination of ischemia alone presages an outcome superior to any attainable using conventional methods. An improved method of organ transplantation combined with an implanted prosthetic disorder response system will eradicate such attempts at repair as halfway measures.

Even though no alternative surgical method can provide as good an outcome, the avoidance of a heart transplant is rooted in the apprehension of rejection, limited durability, and the lifelong need to take immunosuppressives, risking exposure to infection of all kinds. This is equally true whether the heart is severely congenitally malformed in an infant or a younger adult or is at end stage failure later in life.

Also inferior to the compound vascular bypass technique for dealing with a univentricular heart in an infant is a conventional heart transplant, which usually consists not of replacing the recipient heart with an intact heart as the term “heart transplant” would suggest, but rather replacing the ventricles of the recipient heart with those of the donor heart. The ventricles are cut off from both the donor and recipient hearts and those of the donor stitched in place on the heart of the recipient.

After this considerably traumatic operation with the extended use of cardiopulmonary bypass or extracorporeal membrane oxygenation life support, where the graft organ would almost always have been kept for a time unperfused in cold storage to be followed by ischemia-reperfusion injury of the replacement ventricles, the patient is more likely to survive to adulthood but still present cognitive impairment and poor performance in school. While one might presuppose that replacing a defective with a competent heart would bring about a complete cure, the trauma done to both the donor and recipient hearts and to the patient using existing methods for performing a heart transplant remain barriers to a better outcome which metered compound vascular bypass heart transplantation is likely to eliminate.

This is because a metered compound vascular bypass heart transplantation reduces trauma to the unavoidable entry wound in the chest, which is not directly related to the functioning of the heart, and because it imposes the need for adherence to the use of follow-up drugs to include immunosuppressants, elevating susceptibility to infection and imparting much anxiety. Moreover, using current methods, the expectation is that graft failure will follow in ten to twelve years-factors that the constant monitoring and automatic administration of immunosuppressants and other drugs by the fully implanted automatic disorder response system to suppress early signs of rejection, infection, and any of the potential adverse side effects of immunosuppressants is likely to reduce if not completely dispel.

That should reduce a constant apprehension over the probable need for a second heart transplant and the fear of an early death, which distraction, rather than abnormal circulation, is likely the cause for poor performance in school. Both reconstruction and replacement are untenably traumatizing at every level. Using the compound vascular bypass technique, the use of cardiopulmonary bypass can be avoided, regional anesthesia and a sedative used.

By eliminating trauma for the graft organ and the recipient, compound vascular bypass heart transplantation expands the pool of available graft organs. In a pediatric patient, when a heart transplant is needed, reconstruction of the thoracic aorta and the supra-aortic branches when also defective is best avoided by including these in a more inclusive graft. This way the heart and supraaortic branches will grow together. This should still be the preferred approach even if relatively negligible surgery is needed to correct an incision, for example, in the native aorta of the donor.

The alternative, to take only the heart and trust to a patch or hybrid repair, for example, to fix the associated vasculature of the donor admits the problems associated with such repairs in any context. —an inability of the patch proper to grow in step with the patient while at the same time restraining the adjoining tissue from growing as well. If the aorta or other great vessels of both the donor and recipient are unacceptable, a prosthesis that will grow with the patient is described below in section 2b(2)(f), entitled Expandable Structure to Accommodate Growth and Self-adjust in Size in Vascular Segment Prostheses, Lines, and Confluence Conduits Placed in Children, and is shown in FIG. 36F.

Such a prosthesis includes at least one accessory channel into which a drugline can be inserted to allow the direct pipe targeting to it of drugs. With such a prosthesis, a fully implanted disorder response system can be programmed to use sensor feedback to detect adverse conditions and immediately respond to these by dispatching the medication specified in the system prescription-program.

### Treatment of the Heart at End Stage Failure

Associated with adulthood and advancing age, heart failure can actually result from congenital heart defects in infants as to present at birth. The symptoms of heart failure usually include dyspnea, chest pain, edema in the lower extremities, cough, dizziness, fatigue, weakness, bloating, tachycardia, bloated abdomen, nausea, impaired mental alertness, and a rapid increase or decrease in body weight. Progressive, and unless the patient dies from other causes eventually fatal, heart failure demands immediate therapeutic intervention (see, for example, Malik, A., Brito, D., Vaqar, S. and Chhabra, L. 2023. “Congestive Heart Failure,” Online, Treasure Island, Florida: StatPearls Publishing Co.; Mahmaljy, H. Yelamanchili, V. S., and Singhal, M. 2023. “Dilated Cardiomyopathy,” Online, Treasure Island, Florida: StatPearls Publishing Co.).

Stage classifications include that of the American College of Cardiology and the New York Heart Association (see, for example, Large, S. 2007. “Surgery for Heart Failure,” Online, British Medical Journal Heart 93(3): 392-402). Measures that increase circulatory sufficiency, such as the use of a vasodilator, typically nitroglycerin, angioplasty, and/or coronary artery bypass grafting, and valve replacement offer symptomatic relief and usually succeed in extending life. More effective the earlier initiated, conventional treatment of the failing heart to include surgery can improve oxygenation, dispel symptoms, and retard progression of the condition for years.

Surgical intervention includes implanting a pacemaker-cardioverter/defibrillator, and/or a ventricular assist device. The earlier intervention is instituted, the slower will be the continued degeneration of the heart itself and the other organs and tissue dependent upon the heart for oxygen and nutritive delivery. However, over time the disease tends to become refractory to intervention and is can be no more than ameliorated without being cured. In this sense, heart failure is a juggernaut which despite routine measures and undetected and untreated symptoms, as well as due to an accumulation of secondary reactions to the sum of measures undertaken to control the primary disease is likely to progress to end-stage failure.

Surgical operation such as cardiomyoplasty (partial left ventriculectomy, left ventricular reduction) to reduce the size of the enlarged left ventricle in patients with nonischemic dilated cardiomyopathy may leave causative, or etiological, factors intact, and often, but not always, fail (see, for example, Sarohi, V., Srivastava, S., and Basak, T. A. 2022. “A Comprehensive Outlook on Dilated Cardiomyopathy (DCM): State-of-the-art Developments with Special Emphasis on OMICS [the sum of constituents within a cell, to include genomics, proteomics, transcriptomics, and metabolomics]-based Approaches,” Online, MDPI [Multidisciplinary Digital Publishing Institute] Journal of Cardiovascular Development and Disease (Basel, Switzerland) 9(6): 174; Nakamura, K., Uchida, T., Hamasaki, A., and Sadahiro, M. 2020. “Batista Procedure with the Aid of Intraoperative Epicardial Echocardiography,” Online, Brazilian Journal of Cardiovascular Surgery Sao Paulo, Brazil) 35(2): 225-228; Franco-Cereceda, A., Mccarthy, P. M., Blackstone, E. H., Hoercher, K. J., White J. A., Young, J. B., and Starling, R. C. 2001. “Partial Left Ventriculectomy for Dilated Cardiomyopathy: Is This an Alternative to Transplantation?,” Online, Journal of Thoracic and Cardiovascular Surgery 121(5): 879-893).

Left ventricular reduction may be combined with other therapy (see, for example, Letsou, G. V. and Frazier, O. H. 2006. “Successful Ventricular Remodeling with Coronary Artery Bypass Grafting and Mitral Valve Repair in a Patient with Severe Heart Failure,” Online, Texas Heart Institute Journal 33(2): 229-231; Mccarthy, J. F., Mccarthy, P. M., Starling, R. C., Smedira, N. G. Scalia, G. M., and 4 others 1998. “Partial Left Ventriculectomy and Mitral Valve Repair for End-stage Congestive Heart Failure,” European Journal of Cardiothoracic Surgery 13(4): 337-343). Based upon the evidence, a better method for dealing with the heart at end stage failure

Once decompensated and exigent, replacement of the defective heart demands a heart transplant, or rarely, the implantation of an artificial heart. Most often, transplantation will have been avoided long enough for the replacement heart to find itself in a systemically impaired environment. The aversion to transplantation relates primarily to two onerous factors, the lifelong need for immunosuppressive medication that renders the patient susceptible to other disease, to include infection and a heightened risk for cancer.

However, the implementation of a fully implanted automatic prosthetic disorder response system allows the immediate sensor-responsive direct pipe-targeting of immunosuppressives and other drugs directly to the sites where needed, without exposing the rest of the body to harmful side effects, and dispenses the medication automatically, eliminating the prescription nonadherence and the nuisance of having to remember to take the medication. As with use of the compound vascular bypass technique to replace the defective heart in a prenate, neonate, or infant, the trauma is limited to the unavoidable need to create an entry wound; however, the graft organ is spared the trauma and ischemia of a conventional heart transplant.

The scarcity of replacement hearts can be materially alleviated through the approach delineated below in section 2b(1)(b)(b5), entitled Heterotopic Double Heart Metered Compound Vascular Bypass, or Switched-organ Transplantation and the Use of Unified Entry Arterial and Venous Exit Confluence Conduits to Avoid Disuse Atrophy when the Graft Organ is Positioned in the Lower Abdomen by Direct Aortocaval Connection. In the replacement of an anatomically nonanomalous heart at or nearing end-stage failure, to ascertain which vessels between the donor and recipient hearts should be connected as corresponding in order to perform a compound vascular bypass transplant is unproblematic.

This is because the vessels will be alike in number and configuration. However, in a heart so congenitally malformed that direct correspondences thus are lacking or ambiguous, confluence conduits, because these spontaneously apportion flow among the arterial vessels and those venous between the hearts, eliminate the need for studied diagnosis merely to determine these correspondences until the sum volume of blood exiting the kidney is substantially the same as the volume of blood that entered it, and in so doing, materially expedite the connection of arterial-to-arterial and venous-to-venous vessels. Confluence conduits are also addressed in Section 6, entitled Description of the Preferred Embodiments of the Invention, as well as in subsections thereto, to include that entitled Use of Confluence Conduits in the Replacement of a Heart Severely Malformed at Birth.

Vascular ambiguity is encountered in the replacement of kidneys more frequently than with the heart and other major organs. Anomalous (accessory, supernumerary; when polar, aberrant) vessels can differ in number and/or connections. These are usually caused by or have been the cause of disease in the associated kidney such as reflux nephropathy or glomerulonephritis (see, for example, Mukendi, A. M., Rauf, A., Doherty, S., Mahlobo, F., Afolayan, P. and Dawadi, S. 2019. “Renal Arteriovenous Malformation: An Unusual Pathology,” Online, South African Journal of Radiology 23(1): 1704; Yu, L. P., Zhao, W. H., Liu, S., Li, Q., and Xu. T. 2018. “Congenital Renal Arteriovenous Fistula Complicated with Multiple Renal Arteries Malformation: Case Analysis,” Online, (in Chinese with English abstract at Pubmed) Beijing Da Xue Xue Bao Fi Xue Ban [Journal of Peking University (Health Sciences) 50(4): 722-728; Cura, M., Elmerhi, F., Suri, R., Bugnone, A and Dalsaso, T. 2010. “Vascular Malformations and Arteriovenous Fistulas of the Kidney,” Acta Radiologica (Stockholm, Sweden) 51(2): 144-149; additional references relating to supernumerary vessels in kidneys are provided below in section 2b(1)(b)(b8), entitled Metered Compound Vascular Bypass, or Switched-organ, Single and Double Kidney Transplantation. Vascular mal formations other than anomalous (accessory, supernumerary; or aberrant) whether related to the kidneys is addressed below in section 2b(2)(f), entitled Expandable Structure to Accommodate Growth and Self-adjust in Size in Vascular Segment Prostheses, Lines, and Confluence Conduits Placed in Children.

### Transplantation Hesitancy Given an Improved Method for Organ Transplantation and an Implanted Backup Prosthetic Disorder Response System

Due to the greater likelihood of immediate survival, the avoidance of the associated risks of rejection, infection due to immunosuppression, and the development of cancer, and despite the cost in terms of impaired development and lost potential, the choice between inadequate surgical repair and a heart transplant is usually decided in favor of repair. Even though inadequate circulation will result in developmental impairments to include intellectual during the meantime, just so the patient survives for now, the possibility for a heart transplant will continue to be an option once it becomes safer and more durable.

To these legitimate concerns it is responded that the combination of compound vascular bypass solid organ transplantation and a fully implanted prosthetic disorder response system to administer the transfer of the organ, then monitor and automatically and instantly respond to any adverse eventuality in the background, constitutes a cooperative dyad which together realize a method for organ transplantation which is critically superior to the existing convention. Except for the entry wound, which cannot be avoided, the transplantation procedure is fundamentally less traumatic and therefore less likely to trigger degenerative processes such as late-term cardiac allograft vasculopathy and metaplastic transition to malignancy that will make eventual rejection more likely as a cause for constant apprehension.

Instead, silent support should dispel the constant apprehension, distraction, and poor school performance of children who undergo a heart transplant using the technology as it exists. Rejection less likely from the outset, and transplant-responsive immunosuppression directly pipe-targeted, the risks of rejection, infection, and malignancy are materially reduced. The implanted system of sensors and a prescription-program devised to respond to events that might eventuate reacts to adverse feedback instantly and appropriately.

This capability is taken advantage of in using the system to monitor and control the rate of the metered organ transfer from the circulatory system of the donor into that of the recipient. Not only does the system react immediately, silently, and without the awareness of the patient, but it can alert the clinic as to the specifics of the adverse incident instantly.

### Urinary Prostheses

As a prosthetic urine transmission, storing, and drainage system, the lower urinary tract shares more with type item 2 as a breach in the anatomical continuity and physiological integrity of the organism except that it is more differentiated and extensive. Its function is substantially limited to the autonomic and voluntary motor rather than the physiological complexities of participation in electrolyte balance and control over the blood pressure as are the kidneys in the upper urinary tract.

For this reason, whether due to trauma or resection dictated by pelvic evisceration to prevent the further spread of cancer or removal of the bladder responsive to intractable interstitial cystitis (since redesignated chronic bladder pain syndrome) where the surgical construction of a continent bladder would involve the use of gut in a young patient, for example, much as a segment along a ductus which is not in itself a distinct organ with a complex chemical and not just motor function, its complete replacement is best a prosthesis, not a transplant. In contrast to complete resection, incontinence does not justify resection, can often be remedied through a relatively simple surgical procedure, or otherwise through reconstructive surgery using any of several versions of a ‘neobladder’ prosthetic or organic.

Described below is a prosthesis for the lower urinary tract which supported by a prosthetic disorder response system, is intended to remain in place indefinitely without the need for periodic replacement due to an accretion of crystal, for example, as is the case with J-stents. The system bypasses the nerve endings that generate urge sensation in the bladder, so that not only susceptible to malignant degeneration, the patient with overactive bladder, for example, can function without interruption during a public performance whether in delivering a speech or participating in a sport.

Various surgical reconstructions are mentioned in the section below on the urinary system, where it is emphasized that any method which harvests a segment of ileum can first of all involve this as a preliminary procedure subject to all the risks of surgery as well as affect the absorptive character of the ileum as a significant injury at the site of harvesting. Moreover, the site of ileal insertion in the urinary tract will remain susceptible to the erosive effect of urine, possibly leading to metaplastic degeneration and ultimately malignancy.

The shortcomings in autologous tissue transplant procedures are addressed, because the means for urinary diversion delineated below in section 2c, entitled Ductus Side-entry Jackets and Vascular Valves in Urinary Diversion, not only avoid the preliminary harvesting of tissue, but made entirely of synthetic materials, provide the critical benefits of eliminating the need for a blood supply and drainage, nonsusceptibility to infection or metaplastic degeneration, bladder and/or ureteral endometriosis, stenosis, perforateability, prolapse, or any other of the vulnerabilities to which living tissue is subject.

It is the prosthesis itself which is not vulnerable thus, and as addressed below in section 6, entitled Description of the Preferred Embodiments of the Invention, in the subsection thereof entitled Endothelial Function and Epithelialization of Tie-line Type Vascular Prostheses, consideration must be given to the potential for adverse consequences once the lumen becomes epithelialized and might be affected by intimal overgrowth and thrombus. However, even with less suitable synthetic materials, these are, however, controllable using the druglines which can be inserted into the accessory channel or channels always incorporated into side-entry devices.

One of the urinary tract ailments which causes dysuria, or considerable pain during voiding, is refractory intrinsic or extrinsic ureteral endometriosis wherewith the lower urinary tract bypass embodiments shown in FIGS. 28 and 30 can be supplemented with an intracorporeal blood purifier as described in copending application Ser. No. 18/358,252, entitled Fully Implanted Aphereter/Dialyzers for Intracorporeal Blood Purification, to lengthen the interval between successive dialysis treatments in the clinic. The use of these prostheses liberalizes the resection of native tissue used to eradicate the endometriosis lesions where these might otherwise result in a postsurgical recurrence of endometriosis or lead to ovarian cancer, for example.

The implanted dialyzer and lower tract prosthesis incorporate druglines to allow the directly piped targeting of drugs such as relugolix/estradiol/norethisterone acetate, aromatase inhibitors combined with progestogens (progestins) oral contraceptives, or GnRH receptor agonists, and specially prepared 4DryField® PH without the 4DFLap™ applicator, for example, to the affected sites (see, for example, Driva, T. S., Schatz, C., and Haybaeck, J. 2023. “Endometriosis-associated Ovarian Carcinomas: How PI3K/AKT/mTOR Pathway Affects their Pathogenesis,” Online, MDPI [Multidisciplinary Digital Publishing Institute] Biomolecules (Basel, Switzerland) 13(8): 1253; Kramer, B., Andress, J., Neis, F., Hoffmann, S., Brucker, S., Kommoss, S., and Holler, A. 2023. “Improvement in Fertility and Pain after Endometriosis Resection and Adhesion Prevention with 4DryField® PH: Follow-up of a Randomized Controlled Clinical Trial, Online, MDPI [Multidisciplinary Digital Publishing Institute] Journal of Clinical Medicine (Basel, Switzerland) 12(10): 3597; Ngernprom, P., Klangsin, S., Suwanrath, C., and Peeyananjarassri, K. 2023. “Risk Factors for Recurrent Endometriosis after Conservative Surgery in a Quaternary Care Center in Southern Thailand,” Online, PLoS [Public Library of Science] One 18(8): e0289832; Therachiyil, L., Anand, A., Azmi, A., Bhat, A., Korashy, H. M., and Uddin, S. 2022. “Role of RAS [rat sarcoma virus proteins] Signaling in Ovarian Cancer, Online, F1000 [faculty of 1000] Research (London, England) 11:1253; Wilczyński, J. R., Szubert, M., Paradowska, E., and Wilczyński, M. 2022. “Endometriosis Stem Cells as a Possible Main Target for Carcinogenesis of Endometriosis-associated Ovarian Cancer (EAOC),” Online, Online, MDPI [Multidisciplinary Digital Publishing Institute] Cancers (Basel, Switzerland) 15(1): 111; Yachida, N., Yoshihara, K., Yamaguchi, M., Suda, K., Tamura, R., and Enomoto, T. 2021. “How Does Endometriosis Lead to Ovarian Cancer? The Molecular Mechanism of Endometriosis-associated Ovarian Cancer Development,” Online, MDPI [Multidisciplinary Digital Publishing Institute] Cancers (Basel, Switzerland) 13(6): 1439; Murakami, K, Kotani, Y., Nakai, H., and Matsumura, N. 2020. “Endometriosis-associated Ovarian Cancer: The Origin and Targeted Therapy,” MDPI [Multidisciplinary Digital Publishing Institute] Cancers (Basel, Switzerland) 12(6): 1676; Kizilay, Şimşir, A., and Nazli, O. 2018. “Management of Ureteral Endometriosis and Review of the Literature,” Online, Turkish Journal of Urology (Istanbul, Turkey) 45 (Supplement 1): S166-S169; Küçükbaş, M., Kurek Eken, M., ilhan, G., Şenol, T., Herkiloğlu, D., and Kapudere B. 2018. Which Factors are Associated with the Recurrence of Endometrioma after Cystectomy?,” Journal of Obstetrics and Gynaecology (London, England) 38(3): 372-376; Palla, V. V., Karaolanis, G., Katafigiotis, I., and Anastasiou, I. 2017, “Ureteral Endometriosis: A Systematic Literature Review,” Online, Indian Journal of Urology (Mumbai [Bombay], India) 33(4): 276-282; Selçuk, I. and Bozdağ, G. 2013. “Recurrence of Endometriosis; Risk Factors, Mechanisms and Biomarkers; Review of the Literature,” Online, Journal of the Turkish German Gynecological Association (Istanbul, Turkey) 14(2): 98-103; Nezhat, C., Paka, C., Gomaa, M., and Schippers, E. 2012. “Silent Loss of Kidney Secondary to Ureteral Endometriosis,” Online, Journal of the Society of Laparoendoscopic Surgeons (Miami, Florida) 16(3): 451-455; Baldi, A., Campioni, M., and Signorile, P. G. 2008. “Endometriosis: Pathogenesis, Diagnosis, Therapy, and Association with Cancer (Review),” Oncology Reports 19(4): 843-846).

Many conditions and their adverse sequelae would be eradicated were the native tissue replaced by a prosthesis made of synthetic materials without any susceptibility to malignancy or infection (see, for example, Supermainam, S. and Koh, E. T. 2020. “Laparoscopic Partial Bladder Cystectomy for Bladder Endometriosis: A Combined Cystoscopic and Laparoscopic Approach,” Journal of Minimally Invasive Gynecology 27(3): 575-576; Tarumi, Y., Mori. T., Kusuki, I., Ito, F., and Kitawaki, J. 2015. “Endometrioid Adenocarcinoma Arising from Deep Infiltrating Endometriosis Involving the Bladder: A Case Report and Review of the Literature,” Online, Gynecologie Oncology Reports 13:68-70; Mann, S., Patel, P., Matthews, C. M., Pinto, K., an O'Connor J. 2012. “Malignant Transformation of Endometriosis within the Urinary Bladder,” Online, Baylor University Medical Center Proceedings 25(3): 293-295; Balat. O., Kudelka, A. P., Edwards, C. L., Silva, E., and Kavanagh, J. J. 1996. “Malignant Transformation in Endometriosis of the Urinary Bladder: Case Report of Clear Cell Adenocarcinoma,” European Journal of Gynaecological Oncology 17(1): 13-16).

Various procedures, such as the relatively common ileal conduit bladder augmentation procedures, and Mitrofanoff continent appendicovesicostomy, for example, reposition a portion of the gut to a site for the storage and passage of urine. A prosthesis made of synthetic materials requires no blood supply, is unsusceptible to degenerative disease whether infectious, malignant, or any other kind, is more tolerant of inadequate patient hygiene, and can be sterilized with agents such as bleach which should not be applied to the skin.

Malignancy is not the only potential complication of exposing tissue not adapted to contact with erosive urine in such procedures, which also lead to the need for as many as four reinterventions for cutaneous/fascial stenosis and stenosis at the conduit-bladder level, and stomal incontinence necessitating stoma revision, conduit revision, dilatation of a stenotic tract, with most revisions required during the year following construction of the conduit (Babakhanlou, R., Larkin, K., Hita, A. G., Stroh, J, and Yeung, S. C. 2022. “Stoma-related Complications and Emergencies,” Online, International Journal of Emergency Medicine 15(1): 17; D'Ambrosio, F., Pappalardo, C., Scardigno, A., Maida, A., Ricciardi. R., and Calabro, G. E. 2022. “Peristomal Skin Complications in Ileostomy and Colostomy Patients: What We Need to Know from a Public Health Perspective,” Online, MDPI [Multidisciplinary Digital Publishing Institute] International Journal of Environmental Research and Public Health (Basel, Switzerland) 20(1): 79; Tsujinaka, S., Suzuki, H., Miura, T., Sato, Y., and Shibata, C. 2022. “Obstructive and Secretory Complications of Diverting Ileostomy,” Online, World Journal of Gastroenterology 28(47): 6732-6742; Maglio, A., Malvone, A. P., Scaduto, V., Brambilla, D., and Denti, F. C. 2021. “The Frequency of Early Stomal, Peristomal, and Skin Complications,” British Journal of Nursing 30(22): 1272-1276; Reuvers, S. H. M., van den Hoek, J., Blok, B. F. M., de Oliveira Barbosa, T. C., WIffenbuttel, K. P., and Sceepe, J. R. 2017. “20 Years Experience with Appendicovesicostomy in Paediatric Patients: Complications and Their Re-interventions,” Neurourology and Urodynamics 36(5): 1325-1329; Carlsson, E., Fingren. J., Hallen, A. M., Petersen, C., and Lindholm, E. 2016. “The Prevalence of Ostomy-related Complications 1 Year after Ostomy Surgery: A Prospective, Descriptive, Clinical Study,” Online, Wound Management and Prevention 62(10): 34-48).

A propensity for gut to transition to malignancy when exposed to the caustic effect of urine wetting tissue not constituted to tolerate such exposure is seen not only in ileal conduits but in augmentation cystoplasty. Other tissue such as autologous expansion-generated dermis might be less susceptible to such metaplastic degeneration. Carcinoma is the most serious consequence following augmentation gastrocystoplasty, ileocystoplasty, enterocystoplasty, or ileovesicostomy, which harvest a portion of the digestive tract to patch into and enlarge the bladder (see, for example, Comell, C., Khani, F., Osunkoya, A. O., Matoso, A. A. Miyamoto, H., and 3 others 2022. “Secondary Malignancy after Urologic Reconstruction Procedures; A Multi-institutional Case Series,” Human Pathology 119:69-78; Kaseb, H. and Aeddula, N. R. 2022. “Bladder Cancer,” Online, Treasure Island, Florida: StatPearls Publishing Co.; Kitamura, K., Isotani, S., Muto, A., ad Horie, S. 2022. “Efficacy of Pembrolizumab in a Rare Type of Bladder Cancer Arising 25 Years after Augmentation Cystoplasty,” Online, British Medical Journal Case Reports 15(3): e244719; Nip. L., Salmo, E., Surange, R. and Calleary, J. 2021. “Mixed Histology Bladder Cancer as a Complication of Clam Ileocystoplasty,” Online, British Medical Journal Case Reports 14(1): e238818; Boettge, K. R., Paasch, C., Schrader, M., and Strik, M. 2020. “Adenocarcinoma if an Ileal Conduit Developing 8 Years After Cystoprostatectomy for Locally Advanced Prostate Carcinoma,” Online, BMJ [British Medical Journal] Case Reports 13(3): e227015; Soma. T., Yokoyama, M., Uchida, Y., Kirimura, S., Tanaka, H., and 3 others 2020. “Long-term Survival after Radical Cystectomy and Mesenteric Lymph Node Dissection for Squamous Cell Carcinoma Arising from Augmented Bladder with Lymph Node Metastasis: A Case Report,” Online, International Cancer Conference Journal 10(1): 20-23; Tran, T. H. T., Melamed, J., and Deng. F.-M. 2020. “Gastric Adenocarcinoma Arising in Gastrocystoplasty,” Urology 148:270-273; Budzyn, J., Trinh, H., Raffee, S. and Atiemo. H. 2019. “Bladder Augmentation (Enterocystoplasty): The Current State of a Historic Operation,” Current Urology Reports 20(9): 50; Sherman, B. and Taylor, F. 2017. “Adenocarcinoma in a Koff Urinary Ileal Diversion,” Online, Urology Case Reports (Philadelphia, Pennsylvania) 13:126-127; Biardeau, X. Chartier-Kastler, E., Roupret, M., and Phe. V. 2016. “Risk of Malignancy after Augmentation Cystoplasty: A Systematic Review,” Neurourology and Urodynamics 35(6): 675-682; Kono, Y., Terada, N., Takashima, Y., Hikami, K., Hida, T., and 11 others 2016. “Advanced Adenocarcinoma of the Bladder after Augmentation Gastrocystoplasty,” (in Japanese with English abstract at Pubmed), Hinyokika Kiyo (Acta Urologica Japonica) 62(1): 33-37; Reid, S., Althunayan, A., Capolicchio, P., Brimo, F. and Kassouf. W. 2014. “First Case of Invasive Squamous Cell Carcinoma in a Stoma of a Monti Ileovesicostomy,” Online, Canadian Urological Association Journal 8(9-10): E654-E656; Sugishita, K., Mouri, G., Nishimura, Y., Takeuchi, I., Ishizu, A., and Shinohara, N. 2014. “Adenocarcinoma Arising in an Ileum Segment of Scheele's Ring 50 Years after Cystoplasty,” (in Japanese with English abstract at Pubmed), Nihon Hinyokika Gakkai Zasshi (Japanese Journal of Urology) 105(4): 207-211; Husmann, D. A. 2009. “Malignancy after Gastrointestinal Augmentation in Childhood,” Online, Therapeutic Advances in Urology 1(1): 5-11; Husmann, D. A. and Rathbun, S. R. 2008. “Long-term Follow-up of Enteric Bladder Augmentations: The Risk for Malignancy,” Journal of Pediatric Urology 4(5): 381-385; Vemulakonda, V. M., Lendvay, T. S., Shnorhavorian, M., Joyner, B. D., Kaplan, H., Mitchell, M. E., and Grady, R. W. 2008. “Metaplastic Adenocarcinoma after Augmentation Gastrocystoplasty,” Journal of Urology (Philadelphia, Pennsylvania) 179(3): 1094-1096; Castellan, M., Gosalbez, R., Perez-Brayfield, M., Healey, P., McDonald, R., Labbie. A. and Lendvay. T. 2007. “Tumor in Bladder Reservoir after Gastrocystoplasty,” Journal of Urology (Philadelphia, Pennsylvania) 178(4 part 2): 1771-1774; Ali-El-Dein, B., El-Tabey, N., Abdel-Latif, M, Abdel-Rahim, M., and El-Bahnasawy, M. S. 2002. “Late Uro-ileal Cancer after Incorporation of Ileum into the Urinary Tract,” Journal of Urology (Philadelphia, Pennsylvania) 167(1): 84-88; Lane. T. and Shah, J. 2000. “Carcinoma following Augmentation Ileocystoplasty,” Urologia Internationalis 64(1): 31-32; Golomb, J., Klutke, C. G., Lewin, K. J., Goodwin, W. E., deKemion, J. B., and Raz, S. 1989. “Bladder Neoplasms Associated with Augmentation Cystoplasty: Report of 2 Cases and Literature Review,” Journal of Urology (Philadelphia, Pennsylvania) 142(2 part 1): 377-380; Stone, A. R., Davies, N., and Stephenson, T. P. 1987. “Carcinoma Associated with Augmentation Cystoplasty,” British Journal of Urology 60(3): 236-238).

An appendicovesicostomy, which appropriates the appendix to create a conduit leading out of the bladder to a stoma at the body surface, is used less frequently but can pose similar prognoses. Short term sequelae of a Mitrofanoff appendicovesicostomy include stomal stenosis, the need for revision, stone formation, infection, and metabolic consequences (see for example, Brungart, J. G., Miller, C. S., and Schropp, K. P. 2020. “Enterocystoplasty and Appendicovesicostomy in Adults: A Description of Demographics and 30-day Outcomes of Bladder Augmentation,” Online, American Journal of Clinical and Experimental Urology 8(4): 133-139; Veeratterapillay, R., Morton, H., Thorpe, A. C., and Harding, C. 2013. “Reconstructing the Lower Urinary Tract: The Mitrofanoff Principle,” Online, Indian Journal of Urology 29(4): 316-321). An appendicovesicostomy may be used to treat preexisting carcinoma of the urethra, making the ascription of a recurrence in malignancy to the appendicovesicostomy questionable.

Most adverse sequelae, to include malignancy, relate to the irritation and erosive stresses caused by the repeated insertion of a catheter into the surgically created conduit every day. No such factor pertains to the systems described below in section 2c, entitled Ductus Side-entry Jackets and Vascular Valves in Urinary Diversion, and shown in the fixed prosthesis of FIG. 28 where flow diversion jackets 143 and 143′ are not adjustable and the flow path-switchable side-entry valve jacketed embodiment shown in FIG. 30, of which the former is devised to function following the loss or removal of the bladder and ureters.

The fixed, or flow routing nonadjustable, prosthesis shown in FIG. 28 is meant for placement in a patient whose native urinary tract had been removed as irreparably damaged, malignant, or dysplastic, so that there is no longer a native tract to which the flow of urine might be switched. That is, FIG. 28 shows the complete urinary drainage prosthesis as needed for a patient having undergone pelvic evisceration responsive to metastatic cancer or where the urinary tract had become irreparably damaged in an accident.

In the system depicted in FIG. 30, the drainage system can be implanted endoscopically to bypass a retained bladder and/or lower portions of the ureters, and, should the circumstance that necessitated bypass of the naïve tract resolve, the system can also be removed endoscopically. If removed, and the patient has been implanted with a prosthetic disorder response system, then to preserve automated support, the sensors and accessory channels should be retained.

Unlike a native bladder, the neobladder is not emptied by the contraction of its ceiling and walls but rather by the tripping of electrical switch 152 which actuates small turbine 150 to propel urine out of the neobladder once filled. This is of central importance in a prosthesis to be positioned in the pelvis, as it allows the neobladder to be much smaller in size than that native, facilitating its placement as least infringes upon the neighboring anatomy.

Whereas in FIG. 28, prosthetic neoureters 143 and 143′ unadjustably empty into prosthetic neobladder 145, in FIG. 30, flow routing and adjustable valve-jackets also numbered 143 and 143′ have as one object the ability to allow switching between the prosthetic and the native urinary tract, and a second object of making it possible for a public performer to continue uninterrupted despite being affected by an overactive bladder or urinary incontinence, for example. In FIG. 30, flow route switching is controlled by the wearer with push/pull, or Bowden, cables by turning control knobs 153 and 153′.

That control can be motorized to allow adjustment only by the clinician through extracorporeal Bluetooth transmission, for example, to the system microcontroller- or when one arm of a hierarchical control system, the master control microprocessor—is considered obvious. An implanted prosthetic disorder response system can automate the targeted release of medication in coordination with neuromodulatory electrostimulation to subdue pelvic visceral dysfunction to include overactive bladder, urinary, and/or fecal incontinence.

That both OnabotulinumtoxinA and a sacral or tibial electrostimulatory neuromodulator allow the voiding of a larger volume to reduce the intervals between the need to void may not represent the imparting of a virtual cure: the patient may still experience much urethral pain and be kept awake all night as a material impact upon the quality of life. Accordingly, in refractory cases of overactive bladder and interstitial cystitis, the administration of both OnabotulinumtoxinA and sacral electrostimulatory neuromodulation may still fail to improve the quality of life so that case the devices shown in FIGS. 28 and 30, which can or do avoid the bladder remain as a viable alternative. Moreover, druglines 8 and 8′ can be used to automatically deliver medication to ameliorate irritation caused by an electrostimulatory neuromodulator and/or to provide coordinated electrical/medicinal therapy.

The inadequacy of relief in practical terms of current methods extends to urinary dysfunction complicated by additional central nervous system disease (see, for example, Majdinasab, N., Orakifar, N., Kouti, L., Shamsaei, G., Seydedtabib, M and Jafari, M 2023. “Solifenacin versus Posterior Tibial Nerve Stimulation for Overactive Bladder in Patients with Multiple Sclerosis,” Online, Frontiers in Neuroscience (17:1107886; Ghavidel-Sardsahra, A, Ghojazadeh, M., Rahnama'l, M. S., Naseri, A., Yazdandoost, S., and 7 others 2022. “Efficacy of Percutaneous and Transcutaneous Posterior Tibial Nerve Stimulation on Idiopathic Overactive Bladder and Interstitial Cystitis/Painful Bladder Syndrome: A Systematic Review and Meta-analysis,” Neurourology and Urodynamics (New York, New York) 41(2): 539-551; Huang, J., Fan, Y., Zhao, K., Yang. C., Zhao, Z., and 3 others 2022. “Comparative Efficacy of Neuromodulation Technologies for Overactive Bladder in Adults: A Network Meta-analysis of Randomized Controlled Trials,” Neuromodulation S1094-7159(22) 00752-8; Liu, P., Li, Y., Shi, B., Zhang, Q., and Guo, H. 2022. “Comparison of Different Types of Therapy for Overactive Bladder: A Systematic Review and Network Meta-analysis,” Online, Frontiers in Medicine (Lausanne, Switzerland) 9:1014291; De Nunzio, C., Brucker, B., Bschleipfer, T., Cornu, J. N., Drake, M. J., and 7 others 2021. “Beyond Antimuscarinics: A Review of Pharmacological and Interventional Options for Overactive Bladder Management in Men,” European Urology 79(4): 492-504; Chugtai. B., Clemens, J. Q., Thomas, D., Sun, T., Ghomrawi, H., and Sedrakyan, A. 2020. “Real World Performance of Sacral Neuromodulation and OnabotulinumtoxinA for Overactive Bladder: Focus on Safety and Cost,” Journal of Urology (Philadelphia, Pennsylvania) 203(1): 179-184; Lo, C. W., Wu, M. Y., Yang, S. S., Jaw, F. S. and Chang, S. J. 2020. “Comparing the Efficacy of OnabotulinumtoxinA, Sacral Neuromodulation, and Peripheral Tibial Nerve Stimulation as Third Line Treatment for the Management of Overactive Bladder Symptoms in Adults: Systematic Review and Network Analysis,” Online, MDPI [Multidisciplinary Digital Publishing Institute] Toxins (Basel, Switzerland) 12(2): 128; de Groat, W. C., and Tai, C. 2015. “Impact of Bioelectronic Medicine on the Neural Regulation of Pelvic Visceral Function,” Online, Bioelectronic Medicine 2015:25-36).

In FIG. 30, flow diversion jackets 143 and 143′ allow switching between the native and prosthetic effluent paths. Temporary switching thus has the benefit of allowing medicinal or fulgurative treatment, or surgical repair, for example, of the bladder and/or transluminal treatment of either or both ureters while empty of urine. The treatment completed, flow is switched back to the native system. Once the native tract has healed, the prosthetic components can be removed endoscopically. In a stone former or a patient that tends to release crystal that would accrete along the walls of the native ureters or neoureters, the implanted prosthetic disorder system is programmed to release the appropriate crystal solvent through valve accessory channels 8 and/or 8′, which would then automatically be released into whichever drainage system, native or prosthetic was switched into use at the moment.

Another level for switching of urine outflow is at the outlet of the bladder into the urethra. In a female or a prostatetectomized male, urine outflow can be switched between the prosthetic outflow pipe, or neourethra, part number 110 in FIG. 30, and the inlet to the native urethra, to prevent the native urethra from becoming atrophic due to disuse. Whether this should apply in a given patient depends upon the trauma to encircle the native urethra with an inlet jacket and the probable adverse consequences for comfort and reproductive function.

Neither embodiment uses a stoma to cause numerous complications; instead, the outlet portal, of which one embodiment is shown in FIG. 26C, is fully synthetic, and has an antiseptic storage reservoir part number 109 just beneath its outer screw-on outlet protective cap part number 108. Free of living tissue, the device is oblivious to the numerous complications cited above, and does not require the insertion of a catheter through a conduit made of tissue several times a day to push and pull at the tissue causing it to become abraded or stenose. The absence of a rolled gut stoma on the anterior surface of the abdomen also eliminates the numerous complications these contribute due to imitation and infection that to prevent requires constant maintenance.

Moderate (4-9 months) to long-term sequelae of a Mitrofanoff appendicovesicostomy (Mitrofanoff, P. 1980. “Trans-appendicular Continent Cystostomy in the Management of Neurogenic Bladder,” (in French with English abstract at Pubmed) Chirurgie Pediatrique (Pediatric Surgery)(Paris, France) 21(4): 297-305), and similar procedures such as a Yang-Monti ileocecal valve cutaneous catheterizable ileal cecoplasty in a child, or to gain greater length for application in an adult, a Casale, or double Monti spiralized retubularization procedure, (Polm, P. D., de Kort, L. M. O., de Jong, T. P. V. M., and Dik, P. 2017. “Techniques Used to Create Continent Catherizable Channels: A Comparison of Long-term Results in Children,” Urology 110:192-195; Casale, A. J. 1999. “A Long Continent Ileovesicostomy Using a Single Piece of Bowel,” Journal of Urology (Philadelphia, Pennsylvania) 162(5): 1743-1745; Monti, P. R., Lara, R. C., Dutra, M. A., and de Carvalho, J. R. 1997. “New Techniques for Construction of Efferent Conduits Based on the Mirofanoff Principle,” Urology 49(1): 112-115; Yang. W. H. 1993. “Yang Needle Tunneling Technique in Creating Antireflux and Continent Mechanisms,” Journal of Urology (Philadelphia, Pennsylvania) 150(3): 830-834); mostly pertain to the stoma and include “ . . . stromal stenosis, leakage, conduit stricture, angulation of the conduit, and prolapse . . . ” (Faure, A. Cooksey, R., Bouty, A. Woodward, A. Hutson J., O'Brien, M. and Heloury, Y. 2017. “Bladder Continent Catherizable Conduit (the Mitrofanoff procedure): Long-term Issues that Should Not be Underestimated,” Journal of Pediatric Surgery 52(3): 469-472); pelvic organ prolapse, and supravaginal elongation of the cervix with a rectocele (Dorairajan, G., Subbaiah, Bojia. V., and Laigudi, D. N.2021. “Pelvic Organ Prolapse in a Woman with Previous Mitrofanoff Operation: Management of a Case,” Online, Urology Arals 13(3): 305-307); (see also, Veeratterapillay, R., Morton, H., Thorpe, A. C., and Harding, C. 2013, Op cit. preceding paragraph); Hampson, L. A. Baradaran, N., and Elliott, S. P. 2018. “Long-term Complications of Continent Catheterizable Channels: A Problem for Transitional Urologists,” Online, Transplantational Andrology and Urology 7(4): 558-566; Kavanagh, A., Afshar, K., Scott. H., MacNeily, A. E. 2012. “Bladder Neck Closure in Conjunction with Enterocystoplasty and Mitrofanoff Diversion for Complex Incontinence: Closing the Door for Good,” Journal of Urology (Philadelphia, Pennsylvania) 188 (Supplement4): 1561-1565; Casey, R. G., Cullen, I. M., Crotty, T., and Quinlan, D. M. 2009. “Intermittent Self-catheterization and the Risk of Squamous Cell Cancer of the Bladder: An Emerging Clinical Entity?,” Online, Canadian Urology Association Journal 3(5): E51-E52; additional references pertaining to procedures and their short and long- term complications are cited below in section 2c, entitled Ductus Side-entry Jackets and Vascular Valves in Urinary diversion) include a virtually inevitable need for revision due to catheter blockage and/or stomal leakage, which then risk secondary adverse sequelae.

Primary adverse sequelae include “ . . . stomal prolapse, stomal incontinence, channel stricture or stenosis, channel redundancy, false passages, and diverticuli” (Hampson, L. A. Baradaran, N., and Elliott, S. P. 2018, Op cit., just above; (see also Lefèvre, M., Faraj. S., Camby, C., Guinot. A., de Napoli Cocci, S., and Leclair, M. D. 2018, “Appendicovesicostomy (Mitrofanoff Procedure) in Children: Long-term Follow-up and Specific Complications, [in French with English abstract at Pubmed]” Progres en Urologie [Progress in Urology] 28(12): 575-581; Suzer. O., Vates. T. S., Freedman, A. L., Smith, C. A. and Gonzalez, R. 1997. “Results of the Mitrofanoff Procedure in Urinary Tract Reconstruction in Children,” Online, British Journal of Urology 79(2): 279-282).

Providing drug delivery dynamically adjusted responsive to sensor feedback to an implanted microcontroller or microprocessor which controls the drug reservoir outlet pump makes the automatic compensation for inborn errors of metabolism, the treatment of disease, surgical reconstructions, and prostheses far more reliable. In applications involving the diversion of blood, connection of the line from the source to the receiving vessel must meet certain biological requirements or the junction will fail, resulting in internal bleeding, and the direct delivery of a clot preventive drip is essential. In applications involving the diversion of urine, failure of the junction will result in leakage into the pelvic cavity, of which the consequences if not treated immediately can prove grave.

Otherwise, the benefit of a fully implanted fully automatic response system able to dispense medication according to sensor feedback in an ambulatory patient oblivious to the treatment would be in widespread use. While rudimentary and limited to the treatment of a single condition, the appearance of continuous glucose monitors to counteract diabetes presages the automatic diagnosis and administration of insulin that samples and releases insulin directly into the blood, much less directly into the portal vein. That the emergence of an implanted system which reacts immediately with dosage adapted to the severity of the condition as indicated by implant sensors, eliminating the time lost in gaining access to a clinician, is inevitable, should be evident.

### Preservation of Insulin in System Drug Reservoirs

Over an estimated 268 million living adults diabetic, of which 90 percent have type 2, many of whom are dependent upon insulin, the compatibility of insulin with storage at body temperature in a subcutaneously implanted drug reservoir represents a considerable concern. Alternative devices specified here, substances that must be refrigerated such as insulin and stem cells are stored primarily in drug reservoirs equipped with a vacuum jacketed or miniature cryogenic storage dewar and temperature range-preserving insulation liquid nitrogen or a Peltier thermoelectric cooler.

Due to the prevalence of diabetes demanding insulin in the tropics where refrigeration is often unavailable, two approaches-insulation to block out heat and the development of temperature-stable insulin have been developed (see, for example, Jarosinski, M. A., Chen, Y. S., Varas, N., Dhayalan, B., Chatterjee, D., and Weiss, M. A. 2022. “New Horizons: Next-generation Insulin Analogues: Structural Principles and Clinical Goals,” Online, Journal of Clinical Endocrinology and Metabolism (Springfield, Illinois) 107(4): 909-928; Jaiswal, A N. and Vagga, A. 2022. “Cryopreservation: A Review Article, Online, Cureus (Palo Alto, California) 14(11): e31564; Richter, B., Bongaerts, B., and Metzendorf, M. I. 2022. “Thermal Stability and Storage of Human Insulin,” Online, Cochrane Database of Systematic Reviews (Chichester, West Sussex, England) 2022(1): CD015385; Jang, T. H., Park, S. C., Yang, J. H., Kim, J. Y., Seok, J. H., and 4 others 2017. “Cryopreservation and Its Clinical Applications,” Online, Integrative Medicine Research (Amsterdam, North Holland) 6(1): 12-18).

Conventional insulin in vials can be stored without refrigeration for 28 days or 4 weeks at room, but not body, temperature. For storage in a body pack-carried drug reservoir, rather than implanted this would be adequate. Implanted as preferred, the measures specified in the preceding paragraph are necessary. A heat-resistant insulin which does not require refrigeration has been developed, alleviating a significant problem in countries without universal electrical service (Weiss, M. A. 2023. “Real-world Insulin Stability and Global Access,” Lancet Diabetes and Endocrinology 11(5): 307-309. Pendsey, S., James, S., Garrett, T. J., Nord, A. B., Pendsey, S. and 6 others 2023. “Insulin Thermostability in a Real-world Setting,” Lancet Diabetes and Endocrinology 11(5): 310-312; Richter, B., Bongaerts, B., Metzendorf, M. I. 2022. “Thermal Stability and Storage of Human Insulin,” Online, Cochrane Database of Systematic Reviews (Chichester West Sussex, England) 2022(1): CD015385; Farley, A. 2018. “After 35 Years of Dead Ends, Ohio Researcher Develops Heat-resistant Insulin,” Insulin Nation January 2018 at http://insulinnation.com/treatment/after-35-years-of-dead-ends-ohio-researcher-develops-heat-resistant-nsulin/).

Included in a prosthetic disorder response system using a body pack as shown in U.S. Pat. No. 11,759,186, other portable devices capable of keeping insulin cold for up to two full days include a commercially made cooling case of cooling wallet (Taerahkun, S. and Sriphrapradang, C. 2022. “Efficacy of Alternative Cooling Devices Used for Insulin Storage without Refrigeration under Hot-humid Environment,” Online, Annals of Medicine (London, England) 54(1): 1118-1125), and a homemade wallet using acrylate polymer beads and a hand-sewn cotton pouch (Gilligan, M. M., Linnes, J. C., von Oettingen, J. E., and Altenor, K. 2021. “From Toy to Tool: Using Water Beads for Insulin Storage in Haiti,” Pediatric Diabetes (Copenhagen, Denmark) 22(5): 729-733).

The dose of a drug established to be effective in the patient is automatically adjusted in response to sensor data by negative feedback, thus progressing according to an implicit dose response curve. That is, the self-adaptation of the system to the instant condition as reported to the implant microcontroller, or in comorbid disease, microprocessor, consists of increasing the dose in increments within the safe range until the result desired is obtained. If the initial dose is effective, the controller thereafter steps down the dose to the lowest level effective. This dynamic self-adaptive capability is therefore incorporated into the prescription-program as an inherent central factor. By basing dosing on an evaluation of the immediate symptom severity with further treatment based upon the previous result, the system effectively precludes under- or overdosing.

Where an organ has become impaired secondarily in response to a primary defect in metabolism or the malfunctioning of an organ upon which it is dependent, to replace the secondarily affected organ only allows the primary defect to similarly ruin the replacement organ. In such a circumstance, the primary cause of failure must be corrected or controlled. To this end, a fully implanted prosthetic disorder response system can automatically release an essential enzyme to counteract an inborn error in metabolism, for example, plus the system can administer the metered compound vascular bypass transplantation in which the impaired organ is replaced, as well as to continue the metabolic support of the replacement organ after the transplant has been completed. Where the primary cause of failure is another organ, that organ must be replaced first or at the same time as the secondarily impaired organ. A heart-lung transplant is an apt example.

In metered compound vascular bypass, or sudden switched-organ, transplantation, the pattern of blood flow during reciprocal cross-circulation between donor and recipient as schematically depicted in FIG. 15A continues over a time sufficient to cross-microchimerize both donor and recipient. Integral in the bypass process through which the graft organ is seamlessly transferred from the circulatory system of the donor into that of the recipient, the mutual attenuation in adverse immune reactivity by means of microchimerization is fundamentally distinct from the action of immunosuppressive drugs in not impairing the immune system. In FIG. 15A, the straight-line cutoffs on the left-hand donor side indicate that the graft organ has now been severed from the circulatory system of the donor, up to which point blood had continued to circulate through the donor.

FIG. 15A and other drawing figures that show only the diversion chute 18 are intended to clarify the blood flow diversion pattern in as uncomplicated a form as possible with technical details such as valve sidestems 19 omitted. Referring to FIGS. 2, 5, 7, 8, and 10A, it will be seen that the sidestem 19 is behind the upturned distal tip of the diversion chute 18. This should make it clear how the sidestem is oriented when the valve is inverted or reversed in direction. Accordingly, in FIG. 15A, the sidestems are positioned as the initial segments in the confluence conduits, that arterial 9 and that venous 9′ connecting the donor and the recipient. FIG. 15C does indicate the positions of the sidestems.

For this reason, to the extent that chimerization is not facilitated through the administration of immunosuppressive drugs, the recipient is left more compatible with the donor without having been rendered more susceptible to infection and malignancy for the rest of his or her life (see, for example, Shrivastava, S., Naik, R., Suryawanshi, H., and Gupta, N. 2019. “Microchimerism: A New Concept,” Online, Journal of Oral and Maxillofacial Pathology 23(2): 311; Partha, D. and Burlingham, W. J. 2011. “Microchimerism vs. Sensitization,” Current Opinion in Organ Transplantation 16(4): 359-365; Starzl, T. E., Demetris, A. J. Murase, N., Thomson, A. W., Trucco, M., and Ricordi, C. 1993. Donor Cell Chimerism Permitted by Immunosuppressive Drugs: A New View of Organ Transplantation,” Online, Immunology Today 14(6): 326-332).

Microchimerization inherent in the process by which the graft organ is transferred from the donor to the recipient is distinct for that which ensues following transplantation, which applies no less in conventional organ transplantation (see, for example, Mathew, J. M., Leventhal, J. R., and Miller, J. 2011. “Microchimerism in Promoting Graft Acceptance in Clinical Transplantation,” Online, Current Opinion in Organ Transplantation 16(4): 345-352; Starzl, T. E. 1997. “Clinical and Basic Scientific Implications of Cell Migration and Microchimerism after Organ Transplantation,” Online, Artificial Organs 21(11): 1154-1155).

By the same token, the gradual nondestructive mechanism of microchimerism in metered compound vascular bypass, or metered switched-organ, transplantation described below in section 2b(1)(b), entitled Metered Compound Vascular Bypass, or Metered Switched-organ, Transplantation, and the sections to follow, and FIG. 15A should not be allowed to unduly detain the progression of the transfer of the graft organ from the donor to the recipient to completion. Then, despite its destructive mechanism, to clear a more persistent impasse to the continued travel of the servovalve diversion chutes caused by a more tenacious indication of rejection reported to the master control microprocessor by rejection analyte sensors implanted in the donor and recipient, the system is enabled to release an immunosuppressive with its prompt onset of action.

Comprising means for the diversion of blood flow in the vascular tree and of urine in the urinary tract, side-entry flow diversion jackets, valves, and servovalves are unrelated to native or replacement cardiac valves or the blood pooling and varicosity-preventing venous valves (calf muscle pump, peripheral heart) in the lower legs, or crura, and pampiniform plexus, assisted by the diaphragmatic pumping action of respiration, which cooperate to assist in returning blood to the right atrium now understood to exert an active pumping function (Rai, D. B. 2022. Mechanical Function of the Atrial Diastole: A New Discovery: The Motion of Blood in the Venous System-Novel Findings, Boca Raton, Florida: CRC [Chemical Rubber Company Press]; Rai, A. B., Lima, E., Munir, F., Faisal Khan, A., Waqas, A., and 4 others 2015. “Speckle Tracking Echocardiography of the Right Atrium: The Neglected Chamber, Online, Clinical Cardiology (Hoboken, New Jersey) 38(11): 692-697; Rai, D. B. 2013. “The Dynamic Function of the Atrial Diastole of the Heart and Motion of Venous Valves in Humans,” Online, International Journal of Angiology (New York, New York) 22(1): 37-44; Willens, H. J., Fertel, D. P., Qin, J., Labrador, E., and Lowery, M. H. 2008. “Effects of Age and Pulmonary Arterial Hypertension on the Different Phases of Right Atrial Function,” International Journal of Cardiovascular Imaging (New York, New York) 24(7): 703-710; Meissner, M. H. 2005. “Lower Extremity Venous Anatomy,” Online, Seminars in Interventional Radiology (New York, New York) 22(3): 147-156).

The primary object is to provide means for the replacement of organs, to include the heart, and major vasculature so congenitally malformed or defective as to preclude normal development if not pose the risk of early death. In such action, the prescription-program is specific as to the character and degree of the delay and the dose of medication to be used to resume the process of organ transfer. The term ‘bloodline’ denotes a synthetic, usually catheteric, tube used to connect the valves or servovalves on the native or the earlier replaced organ to those on the donor organ. Bloodlines are replete in the drawing figures, those in FIG. 15A shown as part number 9, while those in switchable confluence conduits such as shown in FIG. 32 are labelled part number 174. Bloodlines are immediately identifiable by the blood flowing through them labelled 41.

During a compound vascular bypass transplant, servovalves to feed recipient arterial blood into and receive donor venous blood through passing bloodlines (bloodlines, blood conducting lines) connecting counterpart vessels bloodlines long enough to span the distance connecting counterpart vessels of the donor and recipient before organ harvesting, such as those represented collectively as part number 9 in FIGS. 22A and 22B, can be fused to the bloodline at either end by means of a tenacious friction fit or strongly bonding adhesive.

Other options to assure that the passage of blood is never interrupted-which if not prevented in a heart transplant can cause a life-threatening fibrillation-long to short bloodline switches as described below in section 2b(1)(b)(b13), entitled Bloodline and Drugline Switch Devices: Long when Intercorporeal to Short when Intracorporeal to Accommodate Growth, and the use of a bypass from the sending to the receiving vessel can also be connected by servovalves at either end of a longer length of tubing used as a bypass.

In an infant, the need for surgical reentry due to growth is deferred for years if not nullified for life through the use of the highly expandable fabric described below in section 2b(2)(f), entitled Expandable Structure to Accommodate Growth and Self-adjust in Size in Vascular Segment Prostheses, Lines, and Confluence Conduits Placed in Children, shown in FIGS. 15C, 15D, 36A, and 36F. In FIGS. FIGS. 22C thru 22F (and 17B), the drugline to the inlet side-entry vascular servovalve is shown as part number 8, while the drugline to the outlet servovalve is shown as part number 8′, and flow that continues through the substrate vessel is shown as part number 41, while that diverted is shown as part number 41′.

In FIGS. 22C and 22E, switching blood flow to pass over the allograft anastomoses allows the direct pipe-targeting of these, along with the spleen and lymph nodes of the recipient with immunosuppressants (references cited below in section 2b(2)(1), entitled Compound Vascular Bypass Transplantation-associated and Unassociated Anastomosis Bypasses, in the subsection thereto entitled Means for Promoting the Healing of Anastomoses. A background systemic dose can be used to immunosuppress the marrow (see, for example, Carey, S. T., Bridgeman, C., Jewell, C. M. 2023. “Biomaterial Strategies for Selective Immune Tolerance: Advances and Gaps,” Online, Advanced Science (Weinheim, Baden-Württemberg, Germany) 10(8): e2205105; Lin, G., Wang, J., Yang, Y. G., Zhang, Y., and Sun, T. 2023. “Advances in Dendritic Cell Targeting Nano-delivery Systems for Induction of Immune Tolerance,” Online, Frontiers in Bioengineering and Biotechnology (Lausanne, Switzerland) 11:1242126; Rui, Y., Eppler, H. B., Yanes, A. A., and Jewell, C. M. 2023. “Tissue-targeted Drug Delivery Strategies to Promote Antigen-specific Immune Tolerance,” Online, Advanced Healthcare Materials (Weinheim, Baden-Württemberg, Germany) 12(6): e2202238).

In a nontransplantation context involving the repair or reconstruction of autologous tissue where a vessel had to be transected or where a segment had been severed, or a segment avulsed or crushed as the result of trauma, a simpler and less expensive bypass can be provided as addressed below in section 2b(2)(1), entitled Compound Vascular Bypass Transplantation-associated and Unassociated Anastomosis Bypasses, to include anastomotic bypasses sold as a separate product and FIGS. 22C, 22D, and 22E.

Such consists of a microcontroller chip, side-entry vascular servovalve, and drugline, all implanted subcutaneously and where deeper, through small incisions laparoscopically with a larger incision needed if manual use is necessary to fix the servovalve in position. Such an initially monomorbid application can later be supplemented with additional arms of control as incorporated into a hierarchical control system equipped to deal with plural sites of disorder in a coordinated manner to effectuate the optimal overall homeostatic result for the patient.

To do this decisively demands intervention as soon as possible following birth if not earlier, and the means to be described have been devised to do this without the need to interrupt the flow of blood, or alter respiration, or necessitate general anesthesia. While the heart has been stressed because conventional means for its transplantation are far too traumatizing for a fetus, any solid organ that ultrasonography, for example, reveals to have become malformed should be replaced as early as possible before it causes global secondary defects. Required are novel operative mechanisms and midprocedural monitoring that continuously detects the need for and automatically effects adjustments in medicinal support.

Described and illustrated in the sections to follow, in vascular surgery, side-entry, inline coupling, and inline coupling jacket bridge-mounted diversion jackets, valves, and servovalves make possible the placement of adjustable synthetic shunts and bypasses. In cardiac surgery, vascular side-entry servovalves make possible the diversion and metering for minimally traumatizing solid organ transplantation, to include transplantation of the heart, while inline coupling jacket bridges make possible the repair of major vessel aneurysms, coarctations, and an interrupted aortic arch.

While prenatal transplantation of the fetal heart cannot be accomplished using conventional techniques, fetal aortic valvuloplasty can, and can correct prenatal aortic stenosis that if not corrected would progress to hypoplastic left heart syndrome (Pickard, S. S., Wong, J. B., Bucholz, E., Newburger, J. W. Twortzky, W., and 6 others 2020. “Fetal Aortic Valvuloplasty for Evolving Hypoplastic Left Heart Syndrome: A Decision Analysis,” Circulation. Cardiovascular Quality and Outcomes 13(4): e006127).

In conventional transplantation, the graft organ incurs considerable injury through a loss in perfusion followed by reperfusion injury once the organ is placed in the recipient. To this, conventional heart transplantation adds prodigious additional irreversible incisional injury to both the graft organ and that portion of the native heart to remain in the recipient. In metered compound vascular bypass solid organ heart transplantation, the gradual switching of the circulatory system of the recipient from his native to and through the corresponding organ of the donor gradually switches the donor organ into the circulatory system of the recipient.

Neither heart is otherwise affected until completion of the procedure when the innate organ of the recipient is finally removed from the recipient and the donor organ removed from the donor for placement in the recipient. Throughout this process, both circulatory systems and organs had been supported in optimal living condition with neither incised. This means that at any point in the process, should an exigent circumstance such as an intense rejection reaction arise, transfer of the donor heart can be stopped, and except for the thoracic entry wounds, both hearts and participants will have remained intact.

By contrast, were a midprocedural exigency to occur during a conventional heart transplant, both donor and recipient hearts would have been resected as to make abending and reversing the procedure impossible. Conventional ‘bailout’ would necessitate the immediate availability of an alternative donor heart-essentially an impossibility- or the institution of circulation thorough mechanical means. In a metered transplant, incision into the native heart if any would not take place until the donor heart was positioned in the chest at the completion of the procedure. In exigent situations, procedural reversibility can be critical.

A cardinal consideration pertaining to compound vascular bypass solid organ transplantation is that compared to the corresponding conventional procedure, it is so much less traumatizing on the graft organ and the patient as to make possible a solid organ transplant with a less than optimal graft organ and/or a patient that would otherwise be considered unlikely to survive such a procedure. The patient must still be able to withstand an unavoidable entry wound, but the internal trauma inflicted is comparatively negligible. That in an orthotopic heart transplant the donor heart remains intact makes it possible to moderate more stringent standards for the granting of a cardiac graft organ.

The pool of available hearts would be considerably increased, and the severity of the standard for adults with multiple organs secondarily damaged due to defective circulation and babies with severe congenital heart defects who would ordinarily be declined for a transplant could be eased. Compound vascular bypass double heart heterotopic transplant has the potential to further ease the stringency of graft organ qualification for both the graft organ and the patient, thus further increasing the pool of available organs and transplantable patients.

Section 2b(1)(b)(b5) below, entitled Heterotopic Double Heart Metered Compound Vascular Bypass, or Switched-organ Transplantation and the Use of Unified Entry Arterial and Venous Confluence Conduits to Avoid Disuse Atrophy when the Graft Organ is Positioned in the Lower Abdomen by Direct Aortocaval Connection, includes mention of graft rejection due to the disuse atrophy that results from inadequate perfusion.

However, based upon the precedents cited below in section 2b(1)(b)(b16), entitled Direct Aortocaval Connection: Unified Entry Arterial and Exit Venous Confluence Conduits for the Heterotopic Connection of a Graft Heart in the Lower Abdomen, it appears that disuse atrophy is a problem only when the vessels affected in the transplanted organ are essential. An accessory heart significantly larger than that native or earlier transplanted might make a difference. In a direct aortocaval connection where the lungs are remote and the pulmonary circulation entrusted to the native heart, active use of the pulmonary veins in the transplant, for example, appears unnecessary, where only additional pumping force is sought and oxygenation is entrusted to the native heart, the addition of pulmonary capacity appears to be unnecessary.

That may explain why atrophy of the pulmonary veins appears not to result in the rejection of an accessory heart positioned thus. Instead, the native or earlier transplanted heart and the respiratory centers in the brainstem spontaneously increase oxygenation to compensate for the greater need imposed by the added heart. Based upon space availability, accessory organs such as a heart of once realized an artificial lung of which attempts to provide such are specified below in section 2b(1)(b)(b5), entitled Heterotopic Double Heart Metered Compound Vascular Bypass, or Switched-organ Transplantation [and so on], are more readily positionable in the peritoneal cavity than in the chest.

While it should not impose a demand for pulmonary function significantly greater than would a second heart connected in close adjacent relation to that native on a vessel-by-vessel basis in the chest, to place a second heart in the abdomen where the lungs are remote should result in a gas exchange problem. A standard switched organ heterotopic double heart transplant where the accessory heart is positioned in the abdomen avoids this problem by directly connecting all the vessels between the two hearts to include the pulmonary veins.

This should prod an increase in capacity much as the loss of lung tissue prompts the regenerative development of additional alveoli to compensate for the loss. If dependency upon the pulmonary circulation provided by the primary heart is a concern, the standard heterotopic switched-organ heart transplant with the graft positioned in the abdomen is modified to include additional connections directly to the abdominal aorta and the inferior vena cava. To avoid the need for revision, additional connections are made ab initio. Entry arterial and exit venous confluence conduits are essentially the same but oriented in the opposite direction.

Depending upon the application, however, these will usually vary in the number of bloodlines and valves. Because matching of the participants is carefully done before initiating the procedure, and adjuvant such as immune tolerance induction measures are applied, the need to stop the procedure will more likely be due to a medical exigency other than directly related to rejection. That neither organ experiences a loss in perfusion or incurs injury means that the process can be abended at any time for any reason. Once abended, the relative proportion of opposite participant blood in the circulatory system of either will depend upon the amount of time the process had been allowed to continue before having been ended, each to this extent having been transfused.

In the present context, the term ‘life support’ warrants clarification. The ordinary purpose in conventional life support by a heart and lung machine or extracorporeal venoarterial membrane oxygenator (ECMO), often with breathing tube and ventilator is to maintain circulation and oxygenation of the blood when the heart must be substantially although not completely immobilized. To allow open heart surgery, a reversible cardiac arrest may be induced through injection of a cardioplegic. However, when the heart and/or lungs are to be harvested for compound vascular bypass transplantation, these are not bypassed as in open heart surgery but rather kept functioning in as close to the living condition as possible.

In fact, during cardiac surgery, immobilization of the heart through cardioplegia results in a period of cardiac arrest, anoxia, and a condition of no blood flowing through the heart which can prove harmful to the heart even during reconstructive cardiac surgery using autologous tissue. This factor, along with adverse cognitive sequelae, encouraged the development of off-pump, or beating-heart, cardiac surgery. Most complications associated with cardioplegia arise during open heart and coronary artery bypass graft surgery.

The elimination of anoxia cited as a key benefit in the sudden and metered compound vascular bypass methods of solid organ transplantation made possible by vascular valves and servovalves, the sustainment of normal function to include respiration, body temperature if the blood warmer of the ECMO requires supplementation, metabolism, and endocrine function calls for the use of extracorporeal membrane oxygenation, beat stabilization with the aid of a pacemaker, and medical support to be initiated prior to and then continued once the prospective organ donor has died.

Postmortem sustainment of prospective organ donors has been and continues to be actively studied (see, for example, Fainberg, N. A., Morrison, W. E., West. S., Hasz, R., and Kirschen, M. P. 2022. “Organ Donation from Patients on Extracorporeal Membrane Oxygenation at the Time of Death,” Online, Critical Care Explorations (Philadelphia, Pennsylvania) 4(12): e0812; Palleschi, A., Inci, I., van Raemdor. D. E. Ehrsam, J., Ceulemans, L. J., and 6 others 2022. “Lung Transplantation from Donation after Brain Death Donors on Extracorporeal Support,” Online, Transplantation (Baltimore, Maryland) 106(7): e356-e357; Lazzeri, C., Bonizzoli, M., Guetti, C., Fulceri, G. E., and Peris, A. 2021. “Hemodynamic Management in Brain Dead Donors,” Online, World Journal of Transplantation (Pleasanton, California) 11(10): 410-420; Chang, W. 2018. “Extracorporeal Life Support in Organ Transplant Donors,” Online, Korean Journal of Thoracic and Cardiovascular Surgery (Seoul, South Korea) 51(5): 328-332; Shetty, V. L., Mali, S. S., Shetty, S. V., and Shinde, P. D. 2017. “The Brain-dead Donor: An Anesthesiologist's Perspective,” Indian Journal of Anaesthesia 61(12): 952-956; Kumar, L. 2016. “Brain Death and Care of the Organ Donor,” Journal of Anesthesiology, Clinical Pharmacology 32(2): 146-152; additional references pertaining to the maintaining of potential organ donors on ECMO cited below in this section, as well as in section 2a(3) below, entitled Remotely Procured Graft Organ Preservation, for example).

Newer and relatively recent advancements in mechanical ventilation and life support show promise of reducing the injury to the lungs of extended respiratory support (see, for example, Hu, L., Bonnemain, J., Saeed, M. Y., Singh, M., Quevedo-Moreno, D., Vasilyev, N. V., and Roche, E. T. 2023. “An Implantable Soft Robotic Ventilator Augments Inspiration in a Pig Model of Respiratory Insufficiency,” Online, Nature Biomedical Engineering (London, England) 7(2): 110-123; CTech Feb. 17, 2021. “Sheba and Inspira Technologies Implement Respiratory Treatment for Covid 19 Patients,” Online at 24/7; Israel Hayom Feb. 17, 2021. “Breathe Easy: Sheba Testing Startup's Respiratory Solution for Critically Ill COVID Patients,” Online at israelhayom.com; Ben-Noon, D. and Or Shabtai, A. 2021. “Dual Lumen Cannula and Method of Use,” U.S. Pat. No. 11,541,159, assigned to Inspira Technologies, Ra'anana, Israel; Singh, P. M., Borle, A., and Trikha, A. 2014. “Newer Nonconventional Modes of Mechanical Ventilation,” Online, Journal of Emergencies, Trauma, and Shock (Mumbai [Bombay] India) 7(3): 222-227).

Fostered by inconsistent phenomena such as the continuation of the heartbeat following death, for example, the distinction between alive but comatose and dead has fostered terminology likewise ambiguous. Equating ‘coma’ at any depth with death—of which the literature is replete—is mistaken and confusing. Regardless of confusing terms such as ‘brain death,’ which may be taken as synonymous with ‘irreversibly comatose’ to suggest some retention in brainstem as opposed to any higher function, hemodynamic, endocrine, and metabolic support should be initiated when the dying patient fails to respond to the conventional tests for brain function, shows no sign of conscious awareness, and dead, clearly does not feel pain (see, for example, National Health Service of the UK 2022. “Overview, Brain Death,” Online at nhs.uk/conditions/braindeath; Nguyen, D. 2019. “Evolution of the Criteria of “Brain Death,” A Critical Analysis Based on Scientific Realism and Christian Anthropology,” Linacre Quarterly 86(4): 297-313; Omelianchuk, A., Bernat, J., Caplan, A., Greer, D., Lazardis, C., and 4 others 2022. “Revise the Uniform Determination of Death Act to Align the Law with Practice through Neurorespiratory Criteria,” Online, Neurology 98(13): 532-536; Spears, W., Mian, A, and Greer, D. 2022. “Brain Death: A Clinical Overview,” Journal of Intensive Care 10(1): 16; Nitkin, K. 2017. “The Challenges of Defining and Diagnosing Brain Death,” Online, John Hopkins Medicine; Ad Hoc Committee of the Harvard Medical School to Examine the Definition of Brain Death 1968. “Ad Hoc Committee of the Harvard Medical School to Examine the Definition of Brain Death,” Journal of the American Medical Association 205(6): 337-340).

This support should then be continued until all the organs and tissues of the donor have been harvested. In a compound vascular bypass heart transplant, the donor heart will be gradually transferred from the circulatory system of the donor into the circulatory system of the recipient, literally without missing a beat. Extension in the time the body can be preserved by extracorporeal membrane oxygenation considerable but finite, some additional time may be gained as addressed below in this section through adjuvant direct electrical stimulation of the respiratory centers in the medulla oblongata and pons and the cardiovascular center in the medulla, along with continued support to preserve a normal body temperature, as well as endocrine and metabolic function.

Generally, to sustain the other organs and tissues for transplantation, the heart and lungs should be the last organs harvested. If owing to exigent circumstances, the heart and/or lungs must be harvested before the other organs, the extracorporeal membrane oxygenator is used along with medical support to maintain the suitability of the other organs as long as possible (see, for example, Shetty. V. L., Mali, S. S., Shetty, S. V., and Shinde, P. D. 2017, Op cit. above in this section; Kumar, L. 2016 Op cit. above in this section). The method for sustaining the other organs after the heart and/or the lungs have been harvested is straightforward.

FIG. 15A shows a compound vascular bypass heart transplant which when metered rather than sudden, is at the halfway point in organ transfer between the donor and the recipient with the servovalve flow diversion chutes, or “tongues,” projected halfway into the arterial and venous lumina depicted as collective for simplicity. As the chutes reach the fully open position so that all blood flow is made to bypass the native heart of the recipient and pass instead through the heart of the donor, servovalves above the arterial and below the venous, that is, which straddle to bypass the bypass, simultaneously open as well.

The figure is devised to simplify what are actually multiple vessels and takes advantage of the fiction that all venous flow rises craniad up into the heart upon return when actually the superior vena cava rises a considerable distance above the heart to the origins of the supraaortic arteries before returning blood from the upper half of the body to the right atrium. Additionally for visual clarity, heart-heart blood flow pattern-extraneous elements such as accessory channels and druglines have been omitted.

When the bypass valves fully open, blood flow in the donor bypasses his now fully bypassed native heart. Note that throughout the metered process, the volume of blood through both the recipient and donor hearts is normal, it being the relative proportion of blood from either that comprises the total in each. When the main, that is, the graft organ, arterial and venous servovalves fully close, the graft organ is harvested and the bypass bypassing valves open to bypass donor circulation through the arteriovenous anastomosis now positioned where the donor heart had been.

Oxygenation of the donor having been continued throughout the process by the extracorporeal membrane oxygenator, unless continued over a protracted period, the other organs should have been little affected. An inordinate transfer time would cause some unwanted consequences as specified shortly below; however, the use of vascular valves eliminates such eventualities. If membrane oxygenator availability becomes a problem, an artificial heart and ventilator can be used to continue the perfusion of the other organs of the deceased donor.

The means for accomplishing this involve an application of vascular valves and is explained below. The chief concern is to safeguard the organs from an anoxic episode that would surely detract from their durability following transplantation. Since it is best to harvest the heart last, it can be presumed that the remaining organs will go to other recipients. That is, if a single recipient is scheduled to have the heart and another or other organs replaced, the heart should be that removed last. Before the donor heart is harvested, an increase in mean arterial pressure with the heart continuing to function while an extracorporeal pump has been added as a risk to the alveoli and nephrons, for example, warrants comment.

In contrast to the use of venoarterial extracorporeal oxygenation during cardiogenic shock or cardiac arrest, where it can cause “ventricular distension with pulmonary edema, distal limb ischemia, bleeding, and thromboembolism,” (Choi, M. S., Sung, K., and Yang, H. C. 2019. “Clinical Pearls of Venoarterial Extracorporeal Membrane Oxygenation for Cardiogenic Shock,” Online, Korean Circulation Journal 48(8): 657-677), the uninterrupted functioning of a normal heart not impaired on ECMO as pertains to the heart of a deceased prospective heart donor without cardioplegia should elude these impediments as well as impose a factor of pulsatile or punctuated flow that would tend to counteract adverse consequences. With the deceased prospective heart donor on ECMO, the heart should not present an absolute stroke volume, or ejection fraction that would significantly add to the mean arterial blood pressure. The situation with a brain-dead donor whose heart should continue to beat is probably the generation of a somewhat higher mean arterial pressure than that imparted by ECMO alone.

In any case, heart output that would allow the heart and the rest of the body to deteriorate over an extended period of preservation on ECMO can be decreased or increased to fall within the normal range of 65 to 100 milligrams mercury through the administration of drugs (see, for example, Thet, M. S., Sef, A. V. and Sef, D. 2022. “Can Adequate Hemodynamic Management of Brain-dead Donors Improve Donor Organ Procurement?,” Online, World Journal of Transplantation (Pleasanton, California) 12(4): 79-82; Aboubakr, M., Yousai, M. I. K., and Alameda, G. 2021. “Brain Death Criteria,” Online, Treasure Island, Florida: StatPearls Publishing Co.; Lazzeri, C., Bonizzoli, M., Guetti, C., Fulceri G. E., and Peris, A. 2021. “Hemodynamic Management in Brain Dead Donors,” Online, World Journal of Transplantation,” (Pleasanton, California), 11(10): 410-420; Starr, R., Tadi. P., and Pfeghaar, N. 2021. “Brain Death,” Online, Treasure Island, Florida: StatPearls Publishing Co.; Bein, T., Müller, T., and Citerio, G. 2019. “Determination of Brain Death under Extracorporeal Life Support,” Online, Intensive Care Medicine 45(3): 364-366; Harrar, D. B., Kukreti, V., Dean, N. P., Berger, J. T. 3d, and Carpenter, J. L. 2019. “Clinical Determination of Brain Death in Children Supported by Extracorporeal Membrane Oxygenation,” Neurocritical Care 31(2): 304-311: Lee, H., Cho, Y. H., Sung, K., Yang, J. H., Chung, C. R., Jeon, K., and Suh, G. Y. 2015. “The Use of Extracorporeal Circulation in Suspected Brain Dead Organ Donors with Cardiopulmonary Collapse,” Online, Journal of Korean Medical Science 30(12): 1911-1914; Muralidharan, R., Mateen, F. J., Shinohara, R. T., Schears, G. J., and Wijdiks, E. F. M. 2011. “The Challenges with Brain Death Determination in Adult Patients on Extracorporeal Membrane Oxygenation,” Online, Neurocritical Care 14(3): 423-426)

Here the use of ECMO on an extended basis is recommended where death is imminent or less preferably, just occurred, so that its potentially harmful neurologic effects in a living patient have ceased to be meaningful (see, for example, Li, Q., Shen, J., Lv. H., Liu, Y., Chen, Y., Zhou, C., and Shi, J. 2022. “Incidence, Risk Factors, and Outcomes in Electroencephalographic Seizures after Mechanical Circulatory Support: A Systematic Review and Meta-analysis,” Online, Frontiers in Cardiovascular Medicine 9:872005; Lin, G., Li, Y., Zhuang. Y., Fan Q., Luo, Y., and Zeng. H. 2022. “Seizures in Children Undergoing Extracorporeal Membrane Oxygenation: A Systematic Review and Meta-analysis,” Online, Pediatric Research [in press, 29 Jul. 2022 1-8]; Chiarini, G., Cho. S., Whitman, G., Rasulo. F., and Lorusso, R. 2021. “Brain Injury in Extracorporeal Membrane Oxygenation: A Multidisciplinary Approach,” Seminars in Neurology 41(4): 422-436; Mehta. A., and Ibsen, L. M., 2013. “Neurologic Complications and Neurodevelopmental Outcome with Extracorporeal Life Support,” Online, World Journal of Critical Care Medicine 2(4): 40-47).

That maintaining a brain dead, meaning well and truly dead donor on extracorporeal membrane oxygenation initiated before death is beneficial for organ suitability has been widely validated (see, for example, Bery, A. Marklin, G., Itoh, A., Kreisel, D. Takahashi. T., and 6 others 2022. “Specialized Donor Care Facility Model and Advances in Management of Thoracic Organ Donors,” Annals of Thoracic Surgery 113(6): 1778-1786; Englbrecht, J. S., Lanckohr, Ertmer, C., and Zarbock, A. 2022. “Perioperative Management of the Brain-dead Organ Donor: Anesthesia between Ethics and Evidence,” (in German with English abstract at Pubmed) Online, Anaesthetist 71(5): 384-391; Goldberg, J. B., Levine, A., Ohira, S., Spielvogel, D., and Kai, M. 2022. “Successful Heart Transplantation Recovered from a Brain-dead Donor on Veno-arterial Extracorporeal Membrane Oxygenation Support,” Online, American Society for Artificial Internal Organs Journal 68(4): e64-e65; Gregorini, M., Ticozzelli, E., Avelli, M., Grignano, M. A., Pattonieri, E. F., and 13 others 2022. “Kidney Transplants from Donors on Extracorporeal Membrane Oxygenation Prior to Death are Associated with Belter Long-term Renal Function Compared to Donors after Circulatory Death,” Online, Transplant International 35:10179; Kim, S., Choi, K., Keum, M. A., Kim, M. S., Yoon, S. G., and Kyoung, K. H. 2022. “Evaluation of Red Blood Cell Transfusion Threshold in the Management of Brain-dead Organ Donors,” Online, Medicine (Baltimore, Maryland) 101(50): e32353; Murphy, N., Weijer. C., Chandler, J., D' Aragon, F., Healey, A. Weiss, M. J., and Slessarev. M. 2022. “Best Foot Forward: Now is the Time for Canadian Ethical Guidance on Prospective Interventional Trials of Antemortem Intervention in Organ Donation,” Canadian Journal of Anesthesia 69(10): 1196-1202; Rosen, R. D., Singh, A., and Burns, B. 2022. “Trauma Organ Procurement,” Online, Treasure Island, Florida: StatPearls Publishing Co.; Clarke, C. 2021. “Management of the Brain-dead Organ Donor,” Online, Indian Journal of Thoracic and Cardiovascular Surgery 37 (Supplement 3): 395-400; Dhar, R., Klinkenberg, D., and Marklin, G. 2021. “A Multicenter Randomized Placebo-controlled Trial of Intravenous Thyroxine for Heart-eligible Brain-dead Organ Donors,” Online, BioMed Central Trials 22(1): 852; Srivastava, V., Nakra, M., Shankar, K. A., and Datta, R. 2021. “Optimal Management of Brain-dead Organ Donor,” Online, Medical Journal, Armed Forces India 77(1): 1-5; da Fonseca, Sourza, V. S., Batista, T. O. F., Silva, G. M., Spigolon, D. N., Derenzo, N., and Barvieri, A. 2021. “Strategies for Hemodynamic Maintenance of Potential Brain-dead Donor: Integrative Review,” (English and Portuguese) Online, Einstein (Sao Paulo, Brazil) 19: eRW5630; Dhar, R., Stahlschmidt, E., and Marklin, G. 2020. “A Randomized Trial of Intravenous Thyroxine for Brain-dead Organ Donors with Impaired Cardiac Function,” Online, Progress in Transplantation 30(1): 48-55; Sharma, A., Peltz, M. Wait. M. A., Ring, S. W., Mathur, A., Jessen, M. E., and Murala, J. S. 2020. “The Conduct of Thoracic Organ Procurement,” Asian Cardiovascular and Thoracic Annuals 28(3): 158-167; Anwar, A. S. M. T. and Lee, J.-M. 2019. “Medical Management of Brain-dead Organ Donors,” Online, Acute and Critical Care 34(1): 14-29; Beckman, E. J. 2019. “Management of the Pediatric Organ Donor,” Online, Journal of Pediatric Pharmacology and Therapeutics 24(4): 276-289) Le Blanc C., Genuini, M., Deho, A. Lode, N. Phillippe-Chomette, P. and 5 others 2019. “Successful Extracorporeal Membrane Oxygenation Transport of a 4-month-old Brain-dead Infant for Organ Donation: A Case Report,” Pediatric Transplantation 23(7): e13515; Meyfroidt, G., Gunst, J., Martin-Loeches, I., Smith, M., Robba, C., Taccone, F. S., and Citerio, G. 2019. “Management of the Brain-dead Donor in the ICU: General and Specific Therapy to Improve Transplantable Organ Quality,” Online, Intensive Care Medicine 45(3): 343-353; Sun, X., Qin, K., Dong, J., Li, H., Huang. Y., and 7 others 2017. “The Extra Corporeal Membrane Oxygenation (ECMO) in Deceased Donors after Brain Death with Severe Hemodynamic Instability Allow to Optimize the Viability of Organs Procured for Transplantation,” Online, Transplantation 102: S175; Kang, J. H., Choi, B. H., Moon, K. M., Park, Y. M., Yang, K. H., Ryu, J. H., and Chu, C. W. 2016. “Beneficial Effect of Extracorporeal Membrane Oxygenation on Organ Perfusion during Management of Unstable Brain-dead Donor: A Case Series,” Transplantation Proceedings 48(7): 2458-2460; Maciel, C. B. and Greer, D. M. 2016. “ICU Management of the Potential Organ Donor: State of the Art,” Online, Current Neurology and Neuroscience Reports 16(9): 86; Anderson, T. A., Bekker, P., and Vagefi, P. A. 2015. “Anesthetic Considerations in Organ Procurement Surgery: A Narrative Review,” (English and French), Online, Canadian Journal of Anesthesia 62(5): 529-539; Yang, J. H., Cho, Y. H., Chung, C. R., Jeon, K., Park, C-M., and Suh, G. Y. 2014. “Use of Extracorporeal Membrane Oxygenation for Optimal Organ Donation,” Acute and Critical Care, Online, accjournal.org/m//journal/; de Vries, E. E., Hoogland, P. E. R. Wind, J., Snoejs, M. G. J, and van Heurn, E. L. W. 2013. “Transplantation of Kidneys from Paediatric DCD [cardiac death] Donors: A Comparison with DBD [brain dead] Donors,” Nephrology Dialysis Transplantation 28(1): 220-226; Novitzky, D. and Cooper, D. K. C 2013. The Brain-dead Organ Donor: Pathophysiology and Management, New York, New York: Springer Publishing Co.).

The heart beat persisting for a time past death due to continued functioning of the cardionector (cardionecteur, or conduction system of the heart), and likely the intrinsic cardiac nervous system, heart function in a deceased donor, especially with the support of a pacemaker-cardioverter/defibrillator, may remain stable enough to maintain the organs in transplantable condition to suggest that depending upon the time the graft must be kept in a transplantable condition, maintaining deceased donors on ECMO is usually but not always necessary (Fainberg, N. A., Morrison, W. E., West S., Hasz, R., and Kirschen, M. P. 2022. “Organ Donation from Patients on Extracorporeal Membrane Oxygenation at the Time of Death,” Online, Critical Care Explorations 4(12): e0812; Julian, K., Prichard, B., Raco, J., Jain, R., Jain, R. A. 2022. “A Review of Cardiac Autonomics: From Pathophysiology to Therapy,” Future Cardiology 18(2): 125-133; Gruscheski, L. and Brand, T. 2021. “The Role of POPDC [popeye domain-containing gene family] Proteins in Cardiac Pacemaking and Conduction,” Online, MDPI [Multidisciplinary Digital Publishing Institute] Journal of Cardiovascular Development and Disease (Basel, Switzerland) 8(12): 160; Fedele. L. and Brand, T. 2020. “The Intrinsic Cardiac Nervous System and Its Role in Cardiac Pacemaking and Conduction,” Online, MDPI [Multidisciplinary Digital Publishing Institute] Journal of Cardiovascular Development and Disease (Basel, Switzerland) 7(4): 54; Duraes Campos, I., Pinto, V., Soursa, N., and Pereira, V. H. 2018. “The Brain within the Heart: A Review on the Intracardiac Nervous System,” Journal of Molecular and Cellular Cardiology 119:1-9; Carter, T., Bodzin, A. S., Hirose, H., West, S., Hasz, R., and Maley, W. R. 2014. “Outcome of Organs Procured from Donors on Extracorporeal Membrane Oxygenation Support: An Analysis of Kidney and Liver Allograft Data,” Clinical Transplantation 28(7): 816-820).

Also, post brain death donors having been on ECMO for reasons other than prospective organ donation had usually been more impaired (Bronchard, R., Durand, L., Legeai, C., Cohen, J., Guerrini, P. and Bastien, O. 2917. “Brain-dead Donors on Extracorporeal Membrane Oxygenation, Critical Care Medicine 45(10): 1734-1741). Kidney transplantation using a compound vascular bypass method is addressed below in section 2b(1)(b)(b8), entitled Metered Compound Vascular Bypass, or Switched-organ, Single and Double Kidney Transplantation. However, the preservation of a deceased donor is decidedly unformed and inadequate when sustainment is entrusted solely to ECMO (see, for example, Shetty, V. L., Mali, S. S., Shetty, S. V., and Shinde, P. D. 2017, Op cit. above in this section; Kumar, L. 2016 Op cit. above in this section).

A left ventricular assist device similarly maintains systemic perfusion and favorable hemodynamics during organ retrieval thus protecting prospective graft organs from injury (Loforte, A., Odaldi. F., Berardi, M., Boschi, S., Potena. L., and 5 others 2022. “Multiple Organ Retrieval in a Brain Dead Left Ventricular Assist Device Donor,” Journal of Artificial Organs 25(2): 155-157; Munawar, K., Rajagopalan, N., Grigorian, A., Dennis, D., and Guglin. M. 2020. “Successful Liver Transplantation from a Donor with a Continuous-flow Left Ventricular Assist Device for 9 Months,” Transplantation Proceedings 49(10): 2406-2408).

Most of the literature omits a discussion concerning the mean arterial blood pressure of a subject on ECMO without extraneous factors such as cardiotomy, cardiogenic shock, septic shock, cardiac arrest, or cardioplegia, usually applied to the graft organ as an aid to preservation. FIG. 16 shows the corresponding vessels connected between the recipient and the donor. That the bloodlines connecting corresponding vessels of the donor and recipient number 9, requiring 18 servovalves at either end, or—if the ascending aorta is too crowded to allow its encirclement—12 bloodlines and 24 valves to directly connect the branches of the aortic arch, might be thought that the process is too complex as to invite human error.

However, the process of controlling all of these valves is performed automatically by the master control microprocessor of the prosthetic disorder response control system, with blood flow sensors in the valves to signal any condition of reduced or no flow and manual means provided to control any valve which, albeit improbable, fails to function as intended. The number of valves required means that valves must be as light in weight and as small in size as possible. If necessary because the valves cause discomfort or to reduce the expense of the procedure, the valves are replaced by simple diversion valves described in U.S. Pat. No. 11,759,186, entitled Ductus Side-entry Jackets and Prosthetic Disorder Response Systems.

Once removed, the anastomoses can then be left without bypass. However, this results in the loss of the bypass capability and the targeted release of drugs to the anastomoses or their bypassing to expedite healing, leaving systemic dispersal throughout the body of drugs such as immunosuppressives, anti-inflammatoires, and antimicrobials. The costly servovalves are then sterilized for reuse. While the anastomoses can be left without hardware of any kind, to retain valves in place, each with an internal sensor such as a thin-film strain gauge as pressure sensor and its own accessory channel as a targeting drug delivery line, makes it possible for the implanted prosthetic disorder response system to detect the need for and target medication such as a thrombolytic to any specific anastomosis or combination thereof.

This capability makes it possible to target other drugs such as an immunosuppressive to these sites while sparing the rest of the body increased susceptibility to infection and malignancy. Rather than taken perfunctorily as preventive with dispersion throughout the body, the administration of a statin or steroid, for example, is targeted to the graft organ with the need to place the recipient on a lifelong regimen of bodily dispersed statins and steroids, for example, eliminated. Many deaths due to end stage heart and/or lung disease, these organs are disqualified for transplantation.

Moreover, following the long-term malfunctioning of these organs, other organs dependent upon the heart and lungs to function normally will have been damaged as well. If not, as when the heart failure had begun shortly prior to death so that the kidneys and liver, for example, remain transplantable, the patient is placed on extracorporeal membrane oxygenation support with adjuvant metabolism, endocrine, and body temperature treatment as soon it becomes clear that death is imminent. The glomerular filtration rate preceding death is a key determinant as to transplantability of the kidneys.

The lack of literature pertaining to the use of organs taken from donors affected by heart failure attests to the disqualification of these organs for transplantation (see, for example Shaw, B. I., Sudan, D. L., Boulware, E., and McElroy, L. M. 2020. “Striking a Balance in Simultaneous Heart Kidney Transplant: Optimizing Outcomes for All Wait-listed Patients,” Online, Journal of the American Society of Nephrology 31(8): 1661-1664; Shapey, I. M., Summers, A., Augustine, T., and van Dellen, D. 2019. “Systematic Review to Assess the Possibility of Return of Cerebral and Cardiac Activity after Normothermic Regional Perfusion for Donors after Circulatory Death,” Online, British Journal of Surgery 106(3): 174-180; Damman, K. and Testani, J. M. 2015. “The Kidney in Heart Failure: An Update,” Online, European Heart Journal 36(23): 1437-1444; Snoeijs, M. G., Schaubel, D. E., Hene, R., Hoitsma, A. J., Idu, M. M., and 6 others 2010. “Kidneys from Donors after Cardiac Death Provide Survival Benefit,” Online, Journal of the American Society of Nephrology 21(6): 1015-1021).

In living donor transplantation of a paired organ where the capability exists to switch the circulatory system of the previously well matched recipient from his own to and through the donor organ, the elimination of ischemia and reperfusion injury will increase graft survival and durability. Where, however, the organ is not paired, the object is to continue the circulatory support of the deceased donor, or graft, organ so that it spared the trauma associated with the death of its host. For this reason, the donor is placed on life support which is not disconnected as is the precisely wrong convention. Even if not actually dead, the brain-dead donor is oblivious to the procedure. Exceptionally, either or both the donor and recipient can be placed on hemapheresis to minimize if not eliminate objectionable analytes during the process of organ transfer.

To allow the internal structure of different valves to be shown clearly, the drawing figures show the valves as larger than these are, prompting a misconceived impression of unnecessarily excessive size and weight. Each type valve has also been shown with every element necessary to deal with any potential application. In reality, not all features and the cost of these are likely to be needed in many if not most applications, and where needed are intended to serve a specific purpose.

For example, shown in FIGS. 2, 5, 7, 8, and 10, part number 31 is a lower drug delivery line, or drugline, also referred to as a drugline, drug delivery channel, or accessory channel. As pertains to FIGS. 22B, 32F, and 35F, this feature is of considerable benefit in solid organ transplantation where the servovalves allow switching the flow of blood without interruption from the recipient-donor arterial anastomotic segments and donor-recipient venous anastomotic segments to bypasses that shunt about to omit the anastomotic segments. Formation of the anastomotic bypasses is explanted below in this section as well as in sections 2b (1)(b)(b13), entitled Bloodline and Drugline Switch Devices: Long when Intercorporeal to Short when Intracorporeal to Accommodate Growth, as well as the sections that pertain to these figures.

Sparing the anastomotic segment the constant flowing through of blood leaves it in a relatively nonirritating and dry field wherein inflammation can more readily subside, and which makes it possible to target drugs such as immunosuppressive, anti-inflammatory, and antimicrobial to protect the anastomoses and expedite healing. Moreover, the dry condition can be alleviated periodically through the release of a supportive hydrating, or wetting, agent such as Ringer's lactated or acetated solution which includes dextrose, adenosine triphosphate, as well as drugs to include those of the types just specified. When the heart is harvested first, the remaining organs of the donor are kept continuously perfused as well to best preserve these for transplantation.

This is accomplished by placing a graft organ bypass from a level above the arterial transection for removal to below the venous transection. The bypass is connected at the arterial and venous ends by highly damped nonsparking solenoid-driven valves which remain open to graft organ flow through the graft organ until the graft organ is to be removed. With perfusion maintained by the extracorporeal membrane oxygenator, the valves are closed to divert blood around the direct arteriovenous venous anastomosis. If the ECMO is needed more urgently elsewhere, then connection of the arterial and venous stumps is to the input and output of an artificial heart.

Control of the donor heart bypass solenoid valves as well as the servovalves in the recipient used to switch the flow of blood from passing through the anastomotic bypasses or through the anastomoses is by the fully implanted prosthetic disorder response system master control microprocessor. The formation of such anastomosis bypasses is shown in FIGS. 22B 32A thru 32F, and 35D thru 35F. FIGS. 2, 5, 7, 8, and 10 show vascular servovalves with their diversion chutes fully extended so that all upstream, or oncoming blood flow is redirected out through the valve side stems.

As shown, the release of a drug through the upper surface of the valve sidestem (side stem, side-stem) through drugline, (drugline, drug delivery line, drug delivery and release line, accessory channel), 8′ will release medication through opening 31 beneath the distal tip of the diversion chute into the diverted anastomotic segment to wet the substrate ductus adventitia. Were drugline 8′ extended down through and into the sidestem bore with the diversion chute fully extended into the ductus lumen to fully divert the flow of blood, the medication would empty into the blood diverted to circumvent the anastonosis through the bypass.

Rather than singular as shown, drugline 8′ could be duplicated to provide drug release both against the adventitia as well as to empty into the flow passing out through the bypass. At the same time, with the diversion chute fully extended as shown, the lower drugline, or accessory channel, 8 with outlet pore, or exit hole, beneath its distal tip 31, is situated within to wet the anastomotic segment around which the blood is diverted. Accordingly, with the diversion chute retracted, the control microprocessor can be programmed to release medication through drugline 8′ to empty into the bypass and/or wet the substrate adventitia, whereas with the diversion chute extended, drugline 8 can wet the anastomotic segment.

Applied to FIG. 22B, for example, the valves are positioned preanastomotically to create the inlet into the recipient-donor arterial anastomotic segment at the transplant blood entry and that of the donor-recipient venous anastomotic segment at the transplant blood departure. By the same token, the lower of the valves at the entry and departure from the graft organ represent a second path for the targeting of drugs into the bypassed blood. Regardless how many vascular servovalves a multiple organ compound vascular bypass transplant includes, the coordinated functioning of the drug reservoir outlet pumps leading into the druglines, or accessory channels is administered by the fully implanted prosthetic disorder response system master control microprocessor.

The simplest valve for a given purpose is used so that the complexity and cost for a specific valve will often be less than the drawings suggest. To prevent backfilling with blood and the possibility of obstruction by clot, druglines, or accessory channels, may situate the drugs at the head of a column of water supported by an anticoagulant drip. The implanted controller adjusting the diversion chutes, drug release, backup water filling, and the release of an anticoagulant are coordinated automatically.

Both mainlines (bloodlines) and sidelines (druglines) can be used to supply drugs such as anticoagulants, antimicrobials, antiangiogenics, chemotherapeutics, analgesics, and so on to the side-entry jacket, solenoid or servomotor driven jacket, or nonjacketing side-entry connector to which each is connected. All such lines substantially or completely isolated from one another, different drugs can be separately delivered through any or all, each according to a drug-specific schedule. Complete isolation requires means for preventing entry of any residue into the circulation, for which several methods are delineated below.

Using the bypass techniques addressed herein, neither replacement of the fetal heart nor segments of the great vessels requires interruption in the flow of blood or breathing, and general anesthesia is needed only with a patient who will not cooperate. Side-entry vascular valves also make possible extracardiac transposition of the great arteries without the need for cardiopulmonary bypass, midprocedural cross-clamping, cardioplegia, or general anesthesia, all posing serious risks.

The specific type of vascular valve used dependent upon the application, vascular valves conform to six basic configurations, of which the manually adjusted diversion chute controlled directly by hand if distinguished from that controlled by hand from outside the body by means of a push/pull, or Bowden cable, for example, can be thought of as essentially the same type, or if the distinction is considered to warrant it, that controlled from outside the body can be considered as a seventh type.

The seven configurations are:
1. The bistable, or switched-organ-type, which immediately redirect all flow from either of two outlet passageways into the other or the reverse. There are two types—
   a. Type 1. The plunger solenoid-driven diversion chute such as those shown in FIGS. 7 and 8.
   b. Type 2. The line switch such as those shown in FIGS. 32A thru 32 G, 33A, and 33B. The line switch could be used to transfer flow from a vessel into either of two synthetic tubes gradually in a manner continuously variable, but use thus appearing to lack a practical medical application, this capability is disregarded.
2. The continuously variable switch, which allows the apportioning of flow from a vessel between either of two outlet passageways. There are three types—
   a. Type 3. The servomotor-driven diversion chute such as shown in FIG. 10A.
   b. Type 4. The manually driven diversion chute controlled directly by hand such as that shown in FIG.
   c. Type 5. The manually driven diversion chute controlled directly by hand through a push/pull cable such as shown in FIGS. 5 and 30.
3. Type 6. The servomotor-driven servochoke such as that shown in FIG. 14, which not a switch valve, is able to continuously vary the flow-through cross-section of a vessel in order to adjust the local blood pressure.
4. Type 7. The spring-driven inline coupling jacket such as those shown in FIGS. 36A thru 36D and 37A thru 37D, which like the bistable switch plunger highly damped nonsparking solenoid-driven diversion chute and the line switch is a switch valve able to suddenly transfer all flow, in this case, from an irreparably defective or diseased segment along a native vessel into its prosthetic replacement. However, unlike the plunger solenoid-driven diversion chute and the line switch type valves, which are reversible, or functionally bistable, the inline coupling jacket excises the diseased vessel, eliminating it as an alternative outlet, making reverse or bistable function irrelevant.

Briefly, permanent vascular valves and servovalves allow a tapping off, or diversion, of a portion of the flow through a vessel into a shunt or bypass, whereas inline coupling jackets are vascular valves devised for one-time use to transfer flow from a diseased segment along a vessel with a prosthetic replacement immediately so that the transfer from the native to the tie-line prosthetic lumen is accomplished without a medically significant interruption in the flow of blood.

This factor is crucial when operating on vessels close to the brain and the heart. As indicated, whereas vascular valves and servovalves such as those shown in FIGS. 2, 3, 5 thru 8, 10A thru 10E, 14, 25A, 25B, and 28 thru 30 can redirect a portion or all of the flow into an alternative passageway repeatedly, inline coupling jackets suddenly switch the flow of blood from a segment along a native vessel, into a prosthetic replacement instantly as not to interrupt the flow of blood through the vessel to any noticeable extent.

FIGS. 37A thru 37C show the mechanism positioned between each inline coupling half-jacket at either end of a replacement segment, that is, a tie-line prosthesis positioned between the inline coupling jacket at either end of the tie-line used to transect the defective native segment at either end and immediately rotate the prosthesis into its position. In FIG. 37C, the letter A represents the native diseased artery as the substrate ductus, and the letter S the synthetic prosthesis lumen where the prosthesis is to be rotated around into the position of the substrate ductus. FIG. 36F depicting an aortic arch with supraaortic branches shows that if the segment of the native vessel to be replaced has branches, these are incorporated into the prosthesis and emplaced no differently than a tubular prosthesis.

This wider replacement rather than a conventional focal repair acknowledges that in a child, growth will necessitate revision, and that whether congenital in a child or congenital or acquired in an adult, a defect such as an aneurysm, dissection, coarctation, or interrupted aortic arch will often signify a defect of structure prone to develop similar defects, and that either or both eventualities will necessitate reentry. Better to in the vessel wall so that to replace the wider segment with a prosthesis capable of expansion to adult dimensions and not susceptible to breakdown will once and for all eliminate the need for repeated operations with considerable pain, especially in a child impaired from the outset.

Broadly, defects of the thoracic or the abdominal aorta or another great artery in the young include congenital coarctation, its severe form as an interrupted aortic arch, aortic arch doubling, and congenital abdominal aortic aneurysm, while those in adults are usually the acquired result of atherosclerosis or infection. As addressed below in section 2 (b)(2)(c)(c2), entitled Automatic Insertion into a Ductus of an Inline Coupling Jacket-secured Bridging, or Tie-line, the mechanism to repair an aneurysm, for example, whether congenital in a neonate or acquired in an adult functions automatically, making it possible for a general surgeon to undertake replacement of the defective segment.

FIG. 36A provides a longitudinal section view of a tie-line or bridging vascular prosthesis suitable for the ischemia-free replacement of the native structure without its supraaortic branches whether affected by a congenital or an acquired defect. The embodiment shown incorporates the expansion structure which incorporates flats 233 and vault 234 addressed below in section 2b(2)(f), entitled Expandable Structure to Accommodate Growth and Self-adjust in Size in Vascular Segment Prostheses, Lines, and Confluence Conduits Placed in Children, and is secured at either end by inline coupling jackets addressed below in section 2b(2)(c)(c1), entitled Inline Coupling Jackets Proper, which section also provides the nomenclature for designating the side faces and interfaces of the four jacket halves as depicted in FIG. 36E.

The embodiment shown in FIG. 36F includes the supraaortic branches and is secured at each of its five termini by inline coupling jackets. Using five inline coupling jackets at the termini, this embodiment is automatically placed in the same way as the simpler embodiment shown in FIG. 36A. The automatic process is addressed below in section 2b(2)(c)(c2), entitled Automatic Insertion into a Ductus of an Inline Coupling Jacket-secured Bridging, or Tie-line and Its Replacement If Necessary. FIG. 36B provides a detailed view of the drugline and a through- and-through perforation level beside the interface between the jacket halves adjacent to the automatic transection mechanism of the entry jacket pair shown in FIG. 36C.

In FIGS. 36C, 37E, and 37F, a miniature reciprocating razor saw 226 is used to transect the native segment to be replaced at either end and the prosthesis then rotated into position as shown in FIG. 37A. The process of prosthesis insertion described below in section 2b(2)(c)(c2), entitled Automatic Insertion into a Ductus of an Inline Coupling Jacket-secured Bridging, or Tie-line and Its Replacement if Necessary, once the ends of the native vessel have been transected, the inner half jacket at either end closes around the ends of the stumps securing the prosthesis to the native aorta. The prosthesis is then fixed in position by through- and-through suturing of the outer half-jackets as shown in FIG. 37D, with means for clot prevention specified below.

The speed of reciprocation and the sharpness of the serrated cutting edge of the vessel transection saw 226 are such that any vessel, to include a large muscular artery, will be incised upon contact and not be displaced downward while the saw fails to incise. While most reasons for replacing segments of the great vessels such as the aortic arch or a part of the abdominal aorta do not involve atherosclerotic plaque, this possibility is evaded simply by setting the termini at either end of the prosthetic segment to eradicate the plaque and avoid risking its release as embolizing debris (see, for example, Garcia-Granado, J. F., Galindo-Cordero, A., Hernandez-Perez, S., Perez-Garcia, M. P., Perez-Hernandez, P., and 3 others 2024. “Dysarthria-clumsy Hand Syndrome and Multiple Sequential Acute Limb Embolisms as a Form of Presentation of Aortic Arch Embolism,” (in English and Spanish) Online, Revista de Neurologia (Barcelona, Spain) 79(6): 175-178; Yoshida, Y., Jin, Z., Mannina, C., Homma, S., Nakanishi, K., and 4 others 2024. “Aortic Arch Plaques and the Long-term Risk of Stroke and Cardiovascular Events in the Statin Era,” Online, Stroke (Baltimore, Maryland) 55(1): 69-77; Boyko, M., Chaturvedi, S., Beland, B., Najm, M., Demchuk, A. M., Menon, B. K., and Almekhlafi, M. 2023. “Prevalence of High-risk Aortic Arch Atherosclerosis Features on Computed Tomography Angiography in Embolic Stroke of Undetermined Source,” Journal of Stroke and Cerebrovascular Disease (Philadelphia, Pennsylvania) 32(12): 10737; Jumah. A., Aboul Nour. H., Intikhab. O., Choudhury, O., Gagi, K., Fana, M., and 10 others 2023. “Non-stenosing Carotid Artery Plaques in Embolic Stroke of Undetermined Source: A Retrospective Analysis,” Neurological Sciences (Milan, Italy) 44(1): 247-252; Ntaios, G., Wintermark, M., and Michel, P. 2021. “Supracardiac Atherosclerosis in Embolie Stroke of Undetermined Source: The Underestimated Source,” Online, European Heart Journal (Oxford, England) 42(18): 1789-1796).

Referred to in different contexts, FIGS. 36C and 37C are the same in showing he internal vessel cutting serrated cutting razor reciprocating saw mechanism with lower cutting edge 226 and upper rippled, or wavy, detent surface with depressions 229 to catch the prosthesis upper half-jacket trip. Even when including branches such as the supra-aortic branches of the aortic arch, aortic segment replacement prostheses described herein are inserted automatically and seamlessly without interruption in blood flow, as well as fixed in position by through- and-through suturing of the outer half-jackets as mentioned in the preceding paragraph. These measures and means for eliminating the accretion of clot to the suture traversing the lumen, prevents leaks and migration.

While the tie-line type vascular prosthesis can expand as made according to section 2b(2)(f), the coupling jackets must be fixed in position to avoid migration, and for this reason, allow growth by providing a thickness of jacket foam lining that will accommodate the increase due to growth in the diameter of the substrate vessel. Accordingly, secured at either end by inline coupling jackets and the coupling jacket-tie-line combination devised to accommodate growth, the prosthesis eliminates the need for reentry for many years, in some cases, for life. The procedure and device not only eliminate the need to interrupt the flow of blood through the affected segment but is devised to allow their application by a general surgeon.

Bringing this capability to a considerably larger cohort of practitioners than the specialized cardiac surgeons found only in the largest centers- and in some parts of the world, found not at all-increases accessibility to the repair of these life-threatening defects. Moreover, such repairs carry risks, to include the provocation of a midprocedural desynchronization, such as a ventricular fibrillation, which if not reversed, would result in death. However, the control system entrusted with carrying out the procedure and provided with electrostimulatory pacer-resynchronizers can detect and deliver the shocks necessary to reinstate a normal rhythm.

This application comprises two major parts, the first dealing with valves which capable of redirecting the flow of blood between either of two outlets, allow the replacement of one or more solid organs with no interruption in perfusion, or the redirection of ureteral outflow to bypass the lower urinary tract. The second deals with inline coupling jackets which make possible the automatic replacement of vasculature too malformed or damaged to allow adequate repair without replacement by a prosthesis. The first, dealing with the transplantation of solid organs responds to the fact that organs such as a univentricular heart can be so congenitally malformed or a normal heart can be so damaged that repair sufficient to allow normal function and not just survival is impossible.

Identified over a century ago, microchimerism is ordinary following blood transfusion, especially when repetitive and after the implantation of solid tissue from another individual. As to terminology. “Microchimerization is defined as the presence of two genetically distinct cell populations in the same individual. It can arise from several causes including the bidirectional transfer of cells between mother and fetus during pregnancy, twin-to-twin transfer in utero, from organ transplantation, and blood transfusion” (Matsagos, S., Verigou, E., Kourakli, A., Alexis, S., Vrakas, S., and 9 others 2022. “High Frequency of Post-transfusion Microchimerism among Multi-transfused Beta-thalassemic Patient,” Online, Frontiers in Medicine (Lausanne, Switzerland) 9:845490; see also, Jhelum, H., Bender, M., Reichart. B., Mokelke, M., Radan, J., and 6 others 2023. “Evidence for Microchimerism in Baboon Recipients of Pig Hearts,” Online, MDPI [Multidisciplinary Digital Publishing Institute] Viruses (Basel, Switzerland) 15(7): 1618; Bianchi D. W., Khosrotehrani, K., Way, S. S., Mackenzie, T. C., Bajema, I., and ODonoghue, K. 2021. “Forever Connected: The Lifelong Biological Consequences of Fetomaternal and Maternofetal Microchimerism, Online, Clinical Chemistry (Oxford, England) January 30: 67(2): 351-362; DiGuardo, M. A., Kester, S., Mahaffey, V. J., Hammel, S. A., Heaserr. K. K., and 6 others 2020. “Does Transfusion of Red Blood Cells Impact Germline Genetic Test Results?,” Online, MDPI [Multidisciplinary Digital Publishing Institute] Journal of Personalized Medicine (Basel, Switzerland) 19(7): 2125; Stahlberg, A., El-Heliebi, A., Sedlmayr, P., and Kroneis, T. 2018. “Unravelling the Biological Secrets of Microchimerism by Single-cell Analysis,” Online, Briefings in Functional Genomics,” (Oxford, England) 17(4): 255-264; Hirose, T., Tanaka, Y., Tanaka, A., Sakai, H., Sasaki, Y., Shinohara, N., and Ohdan, H. 2017. “PD-L1/PD-L2-expressing B-1 Cells Inhibit Alloreactive T Cells in Mice,” Online, PLoS [Public Library of Science] One (San Francisco, California) 12(6): e0178765; Shen, X. F., Jiang, J. P., Yang, J. J., Wang, W. Z., Guan, W. X. and Du, J. F. 2016. “Donor-specific Regulatory T Cells Acquired from Tolerant Mice Bearing Cardiac Allograft Promote Mixed Chimerism and Prolong Intestinal Allograft Survival, Online, Frontiers in Immunology (Lausanne, Switzerland) 7:511; Bloch E. M., Jackman, R. P., Lee, T. H., and Busch, M. P. 2013. “Transfusion-associated Microchimerism: The Hybrid Within,” Online, Transfusion Medicine Reviews (Orlando, Florida) 27(1_: 10-20; Knippen, M. A. 2011. “Microchimaerism: Sharing Genes in Illness and in Health” International Scholarly Research Notices 2011:893819; Gammill, H. S. and Nelson, J. L. 2010. “Naturally Acquired Microchimerism,” Online, International Journal of Developmental Biology (Vizcaya, Spain) 54(2-3): 531-543; Du, J. F., Wang, C., Wang, W. Z., Li, M. B., Liang, H. L., and Guan, W. X. 2009. “Transferable Cardiac Allograft Acceptance Induced by Transfusion of Donor B Cells with Impaired Inducible Costimulator/B7h Allorecognition,” Transplantation Proceedings (New York, New York) 41(5): 1840-1843; Reed, W., Lee, T. H., Norris, P. J., Utter, G. H., and Busch, M. P. 2007. “Transfusion-associated Microchimerism: A New Complication of Blood Transfusions in Severely Injured Patients,” Seminars in Hematology (Philadelphia, Pennsylvania) 44(1): 24-31; Utter, G. H., Reed, W. F., Lee, T. H., and Busch, M. P. 2007. “Transfusion-associated Microchimerism,” Vox Sanguinis (Oxford, England) 93(3): 188-195; Starzl, T. E. 1997. “The Basis of Allograft Acceptance,” Online, Forum (Genova, Italy) 7 (Supplement 6): 7-18; Rao, A. S., Thomson, A. W., Shapiro, R., and Starzl, T. E. 1994. “Chimerism after Whole Organ Transplantation: Its Relationship to Graft Rejection and Tolerance Induction,” Current Opinion in Nephrology and Hypertension 3(6): 589-595).

One object in metered compound vascular bypass solid organ transplantation to be described is to accomplish an incipient measure of immune tolerance induction during the reciprocal cross-transfusion intrinsic in the compound vascular bypass procedure for transferring the graft organ from the circulatory system of the deceased donor on life support into that of the recipient. When necessary, apheresis is begun as far in advance of the initiation of reciprocal cross-transfusion as possible and continued during and following transfer of the graft organ.

In neither sudden nor metered transplantation is the postoperative administration of other tolerance immune induction biologicals and drugs when feasible discouraged (see, for example, Bedair, B. and Hachem, R. R. 2021. “Management of Chronic Rejection after Lung Transplantation,” Journal of Thoracic Disease 13(11): 6645-6653, Worel, N. Taleghani, B. M., and Strasser E. 2019. “Recommendations for Therapeutic Apheresis by the Section “Preparative and Therapeutic Hemapheresis' of the German Society for Transfusion Medicine and Immunohematology,” Transfusion Medicine and Hemotherapy 46(6): 394-406).

Microchimerism in blood transfusion can also prove adverse in provoking genetic mismatch-based adverse activation of the immune system, so that stringent as practicable donor-recipient matching should be established prior to initiating the organ transfer procedure. Especially in sudden compound vascular bypass, or sudden switched-organ organ transplantation, primarily intended for use in emergencies, the donor and/or recipient blood may require to be processed by an apheresis machine to eliminate mismatched human leukocyte antigen donor-recipient blood components that would result in adverse reactivity.

Another implementation of the sudden switch technique is in making possible unprecedented solid organ transplantation in prenates and neonates, especially of the heart, where the immature and weak immune system of the system of the patient is less intolerant of alien tissue. Addressed below in section 2b(1)(b)(b2), entitled Prenatal Compound Vascular Bypass, or Switched-organ Transplantation of the Heart, sudden switch transplantation follows matching and the perioperational, that is, the preoperative, intraoperative, and postoperative, use of immune tolerance inducing agents.

With one extracorporeal apheresis machine whether hemapheresis or photopheresis connected to the donor and/or recipient pre- or postprocedurally, or extracorporeal apheresis or photopheresis machines simultaneously and separately connected to the donor and/or recipient apart from the lines connecting the two to accomplish reciprocal cross-transfusion, incompatible blood components can be selectively removed from the blood of either or both, with or without the concurrent administration to either or both of immune tolerance-inducing and immunosuppressive drugs. The automatic disorder response system is programmed to detect deviations in blood pressure due to volume shifts during leukapheresis, for example, and apply remedial action as necessary.

The removal of a defective organ for replacement is neither “harvested,” which term denotes the procurement of a graft organ or tissue for transplantation, nor having never been implanted, is it ‘explanted.’ It is simply removed. Even though actually subsurfacial, drug replenishment ports implanted subdermally, or subcutaneously—usually in the pectoral region—are referred to as ‘body surface ports.’ An original site of disease such as a ‘mother’ neoplasm is that ‘primary,’ as opposed to a secondary or metastatic one.

Conventionally used to denote the initial site of colonization or the nest of pathogens, the term ‘nidus,’ is used to denote the origin of primary site within the body from which disease had spread. References not cited for direct relevance were chosen as indicative of avenues for future development. For example, as initially presented, paper-based, lab-on-a-chip, and microfluidic devices incorporate analytical techniques that suggest reembodiment in the form of implantable sensors which are directly applicable to the objects sought. Compound vascular bypass, or sudden switched-organ transplantation, especially metered compound vascular bypass, or sudden switched-organ, transplantation, effects the transfer of the graft organ directly from the circulatory system of the donor into the circulatory system of the recipient with no interruption in perfusion.

It does this through a process of continuous donor-recipient reciprocal cross-circulation to achieve a measure of donor-recipient immune tolerance-inducing microchimerization which even though its effect may take place after an interval following the procedure, dulls the immunological shock of confrontation with alien tissue. Heart transplantation accomplished thus eliminates the trauma and hypoxia of conventional heart transplantation which limits the durability of the procedure, and in a neonate born with a severely, even unsurvivably malformed heart, overcomes the restraint on normal development of a repair which cannot institute normal pulsatile blood flow.

By directly transferring the graft organ from the life support-sustained circulatory system of the deceased donor into the circulatory system of the recipient, compound vascular bypass organ transplantation achieves a simplicity consisting of eliminating the numerous steps and conditions which ensue before the organ is even harvested and are precisely those which impair the organ and reduce its fitness and viability. These include the loss of oxygen while remaining in the deceased donor, the shock of excision during harvesting, often storage in a cold static or flowing perfusate and time delay during transport, bisection, and in a heart transplant, extensive suturing to the retained portion of the recipient heart.

That using a switched-organ technique leaves both the recipient and the donor hearts intact comprehends options for ‘bailout’ that cutting each in two does not; despite the routine preliminary check of the donor heart upon receipt, resection of most of the recipient heart in preparation to receive the complement from the donor heart can mean that should the graft for any reason prove inadequate, there is no ‘going back.’ Postprocedurally, that either heart can be controlled without the need for reentry makes possible options for the use of either to compensate for a deficit in the other, which eventualities can be incorporated into the prescription-program to proceed automatically.

Furthermore, just as replacing the severely defective heart in a prenate or neonate truncates the secondary degradation in development of the rest of the body due to defective circulation, the same applies to the failing support of the rest of the body by a failing heart in an adult, as witness cardiorenal, cardiohepatic, and cardiopulmonary disease. Compare, for example, the consequences of nonpulsatile flow following repair in an infant with the degradation in an adult secondary to diastolic dysfunction, or heart failure whether with preserved ejection fraction, or heart failure with systolic dysfunction, or a loss in ejection fraction. The numerous adverse effects of nonpulsatile, or pulseless, flow on a patient, especially one pediatric is addressed below in this section and section 2a(1), entitled Congenital Defects of the Heart and Other Organs—The Importance of Prompt Intervention

In either case, the sooner the defective circulation is reversed by replacing the defective heart which is its source, the sooner will the secondary deterioration and the loss in quality of life the defective circulation had caused to include dyspnea, edema, elevated jugular venous pressure, and fatigue, be stopped. The same may be said for the essential replacement of all the major organs, to include the kidneys, liver, lungs, and glands. At any stage in life, deferring a transplant necessitated by an irreparable if not progressive condition not only defers the restitution of the patient to a more normal life but stops the secondary degradation which global, would continue to make the patient even sicker.

And because the patient will 1. Continue to become more impaired, 2. Eventually require a transplant when even weaker, 3. Have to take drugs anyway, probably more drugs at higher doses, and 4. A fully implanted prosthetic disorder response system can automatically monitor and respond to the need for medication, the assertion that an organ transplant only exchanges one disease for another is not just insidious but false. Deferring a transplant can necessitate the use of drugs not significantly less dangerous than are immunosuppressives.

For example, deferring a heart transplant before reaching acute decompensated end stage congestive heart failure with peripheral edema, plural effusions, and swelling of the lower extremities as well as dyspnea necessitates the use of a diuretic. The most frequently used diuretic is furosemide, or ethacrynic acid, recognized for over a half century as a potent ototoxin capable of causing irreversible hearing loss (Rybak, L. P. 1993. “Ototoxicity of Loop Diuretics,” Otolaryngology Clinics of North America 26(5 (: 829-844; Quick, C. A. and Hoppe, W. 1975. “Permanent Deafness Associated with Furosemide Administration,” Annals of Otology, Rhinology, and Laryngology 84(1 Part 1): 94-101; Vargish, T., Benjamin, R., and Shenkman, L. 1970. “Deafness from Furosemide,” Annals of Internal Medicine 72(5): 761). Conventional kidney transplant recipients report that compared to the onerous demands of dialysis, taking immunosuppressives is a radical improvement in quality of life, and an implanted automatic disorder response system would remove the nuisance and risk in the use of an immunosuppressant.

Unlike kidney, liver, and lung transplantation, which allow one of a pair or portional organ transplantation with tissue harvested from a living as opposed to a deceased donor, the singular heart affords little in the way of opportunities to gauge the consequences of insults incurred during transplantation, such as the duration of anoxia and the condition of no blood flow. Unless the donor is located at a great distance so that the graft kidney must be transported to the location of the prospective recipient, live kidney transplantation minimizes the trauma inflicted on the graft organ prior to implantation.

Harvesting the graft organ immediately upon the death of the donor and immediately implanting it in the recipient would probably reduce the difference in durability somewhat; however, anoxia and disembodiment of any duration certainly reduce durability in proportion to the time lost. In heart transplantation, these factors do not account for the trauma of bisection and extensive use of suture that would add to the midprocedural insults to injure both graft and innate organs much more and further reduce the likelihood for survival over a time greater than when these actions are eliminated, which is precisely what compound vascular bypass organ transplantation is intended to accomplish. Furthermore, the donor deceased but without having undergone a period of circulatory stasis, the potential for injury to a living donor is eliminated.

The microchimerization imparted during metered compound vascular bypass living donor kidney transplantation makes it possible to reduce the use of immunosuppressives associated with the increased post-transfer risk of cancer (see, for example. Sprangers, B., Nair, V., Launay-Vaher, V., Riella, L. V., and Jhaveri, K. D. 2018. “Risk Factors Associated with Post-kidney Transplant Malignancies: An Article from the Cancer-Kidney International Network,” Clinical Kidney Journal 11(3): 315-329; Zilinska, Z., Sersenova, M., Chrasrtina, M., Breza, J. Sr., Bena, L. and 7 others 2017. “Occurrence of Malignancies after Kidney Transplantation in Adults: Slovak Multicenter Experience,” Neoplasma 64(2): 311-317; Mynarek, M., Hussein, K., Kreipe, H. H., and Maecker- Kolhoff, B. 2014. “Malignancies after Pediatric Kidney Transplantation: More than PTLD [post-transplant proliferative disease]?,” Pediatric Nephrology 29(9): 1517-1528; Marcen, R. 2009. “Immunosuppressive Drugs in Kidney Transplantation: Impact on Patient Survival, and Incidence of Cardiovascular Disease, Malignancy, and Infection,” Drugs 69(16): 2227-2243).

The incidence of cardiovascular death after kidney transplantation appears to be neutral to the technique of-transplantation, associated instead with advanced age and generally ill health of the recipient, diabetes mellitus, and preexisting cardiovascular disease (see, for example, Awan, A. A., Niu, J., Pan, J. S., Erickson, K. F., Mandayam, S., and 3 others 2018. “Trends in the Causes of Death among Kidney Transplant Recipients in the United States (1996-2014)” American Journal of Nephrology 48(6): 472-481; Pilmore, H., Dent, H. Chang, S., McDonald, S. P., and Chadban, S. J. 2010. “Reduction in Cardiovascular Death after Kidney Transplantation,” Transplantation 89(7): 851-857; Howard, R. J., Patton, P. R., Reed, A. I., Hemming, A. W., van der Weft, W. J., and 3 others 2002. “The Changing Causes of Graft Loss and Death after Kidney Transplantation,” Transplantation 73(12): 1923-1928).

Other factors such as the age, health, and gender of the donor and the degree of genetic (human leucocyte antigen) matching and ABO blood typing aside-over a period of decades, living, rather than deceased donor kidneys have proven more durable as well as less susceptible to the transmission of disease (see, for example, Callaghan, C. J. and Marks, S. D. 2020. “Utilization of Kidneys from Deceased Donors at Increased Risk of Infectious Disease Transmission: A step in the Right Direction,” Pediatric Nephrology 35(2): 177-179; Sigurjonsdottir, V. K. and Grimm, P. C. 2019. “Living or Deceased Donor Kidney Transplantation in Children,” Current Opinion in Pediatrics 31(2): 232-236; Ma, M. K., Lim, W. H., Turner, R. M., Chapman, J. R., Craig, J. C., and Wong, G. 22014. “The Risk of Cancer in Recipients of Living-donor, Standard, and Expanded Criteria Deceased Donor Kidney Transplants: A Registry Analysis,” Transplantation 98(12): 1286-1293; Nemati, E., Einollahi, B., Pezeshki, M. L., Porfarziani, V., Fattahi, M. R., 2014. “Does Kidney Transplantation with Deceased or Living Donor Affect Graft Survival,” Nephro-urology Monthly 6(4): e12182; Premasathian, N., Vongwiwatana, A., Taweemonkongsap, T., Amomvesukit, T., Limsrichamrem, S., and 9 others 2010. “The 37-year Kidney Transplantation Experience at Siriraj Hospital,” in Clinical Transplants 2010, Terasaki Research Institute: Los Angeles, California; Naderi, G. H., Mehraban, D., Kazemeyni, S. M., Darvishi, M., and Latif, A. H. 2009, “Living or Deceased Donor Kidney Transplantation: A Comparison of Results and Survival Rates among Iranian Patients,” Transplantation Proceedings 41(7): 2772-2774).

vascular bypass kidney transplantation, primarily because it eliminates excision and anoxia, is superior to conventional living, much less deceased, donor kidney transplantation. In compound vascular bypass kidney transplantation, there is no equivalent to either a living or deceased donor kidney transplantation, both of which follow an interval of ex vivo, or extracorporeal, anoxia. Rather, normothermic perfusion is maintained past death without an ex vivo anoxic interval allowed to intervene before implantation. Contrary findings indicating relative parity of outcome between conventional living and deceased donor kidney transplantation have been reported but without indicating the ex vivo anoxic interval following harvesting and storage if any with a living donor, and follow-up is seldom reported for a period of many years for both living and deceased kidneys pertinent to pediatric patients.

However, even in conventional living donor kidney transplantation, a shorter preprocedural duration of a deceased donor kidney in normothermic circulating storage is more favorable for the outcome than is storage in a static or flowing cold perfusate, and the trauma imposed on a living donor kidney is considerably less traumatizing than is the trauma imposed on both the donor and replacement hearts (Zhang, X., Lyu, J., Yu, X., Wang, L., Peng, W., Chen, J., and Wu, J. 2020. “Comparison of Graft Outcome between Donation after Circulatory Death and Living-donor Kidney Transplantation,” Transplantation Proceedings 52(1): 111-118).

Deceased donor age over 50 appears more significant than a difference between one and two days in cold storage (Lee, C. M., Carter, J. T., Randall, H. B., Hiose, R., Stock, P. G., and 4 others 2000. “The Effect of Age and Prolonged Cold Ischemia Times on the National Allocation of Cadaveric Renal Allografts,” Journal of Surgical Research 91(1): 83-88). Broadly, absent a well-matched living donor, a kidney from a deceased donor less than 55 years old stored for no more than a day, preferably not in a static or flowing cold perfusate, should be used.

In the drawing figures directed to compound vascular bypass organ transplantation, the norm has been adopted that, the recipient organ is shown on the right and the donor, or graft organ, on the left. Arterial supply is shown above the organs and venous drainage below. Accordingly, arterial blood flow is from the circulatory system of the recipient—from right to left and venous flow below from left to right. In most of the drawing figures, such as FIGS. 32A thru 32C, a measure of simplification and visual clarity has been attained by showing only the venous return from the donor organ moving to the right into the circulatory system of the recipient. Were the arterial supply shown as well, flow therethrough would be from the right to the left above the organs, the arterial flow simply the reverse of the venous. To be certain this is clear, in FIG. 35C, the arterial flow has been shown as well as the venous.

Confluence conduits are common channels to which plural arterial or venous bloodlines are connected to flow together. A confluence conduit usually has a luminal, or flow-through, volume delivery rate in milliliters per second equal to the sum of the volume delivery rates of the bloodlines connected to it. Two dimensional, however, face-on, or full-face, drawing figures as best depict the connections involved lack depth. For this reason, the confluence conduits shown in FIGS. 35A thru 35F have been depicted as at least three times greater in diameter.

Common channeling facilitates organ transplantation using a sudden or metered compound vascular bypass, or switched-organ, transplantation method, the latter denoting the gradual transfer of the graft organ from the circulatory system of the donor into that of the recipient in order to allow an initial measure of immune tolerance induction to blunt the shock of confrontation with alien tissue. Facility gained through the use of confluence conduits includes the following:

1. Reducing the number of bloodlines that must be kept track of during the procedure.

2. Allowing the subset of bloodlines segregated by connection to the same confluence conduit or switchable confluence conduit, such as between those supplying or draining the upper body through one, and those supplying or draining the lower body through another, to be medicated through one or more accessory channels or otherwise treated differently than vessels not included in a confluence conduit.

3. Serves as an additional point for the microchimerization through blending of the blood of donor and recipient whether the effect is augmented by placement of a small inline pump inside the confluence conduit, part number 167 in the drawings, which can also adjust the pressure of the blood moving through it and assist in propelling the blood to traverse a long cross-circulation bloodline needed to span from the donor organ to the recipient. Such a pump can also be positioned inside a bloodline (mainline) or drugline (sideline) to propel, retard, withdraw, or aspirate the fluid passing through it or having settled. An inline pump is substantially limited to positioning inside the longer of two switchable lines or conduits which as shown in FIGS. 32A thru 32C will be discarded rather than implanted as would otherwise interfere with pulsatile flow.

4. Spontaneously apportions, or cross-levels, the volume of flow among vessels to and from the graft and native organs despite a difference in their number or caliber when either or both present supernumerary (accessory, anomalous, or if polar, aberrant) vessels as may be encountered in kidney transplantation.

5. Allows connection of a heterotopically positioned graft organ, such as a infrarenally or iliac placed kidney or heart to nearby vessels differing in number from those of the graft organ. Adjustments in pressure if necessary are accomplished by tap-off vascular servovalves feeding into large nearby vessels, inotrope drip, through the conduit drugline, or accessory channel, part number 8 in the drawings, and several other methods, all automatically administered by the implanted disorder response system.

FIGS. 32A thru 32C, 33A, and 33B depict the mechanism used to implement switchable bloodlines and switchable confluence conduits. As indicated by the difference between FIG. 35A which shows a switchable bloodline and FIG. 35B which shows a switchable confluence conduit, because an inline axial pump 167 positioned in the lumen would block the propagation of pulsatile flow past it, known especially in a younger patient to prove developmentally injurious as is nonpulsatile Fontan circulation, the use of such a pump is limited to the longer disposable of the two switchable bloodlines or switchable confluence conduits which as shown in FIG. 32B is in place only during the compound vascular bypass transfer of the graft organ from circulatory system of the donor into that of the recipient.

Showing the venous arm is preserved in the depiction of confluence conduits even though it is often the arterial flow which requires flow between graft organs that differ in their number of arteries as arises most often in the transplantation of a kidney where either the donor or recipient graft organ has an anomalous supernumerary (accessory, anomalous, or if polar, aberrant) arterial supply. This is compensated for in FIGS. 35A and 35B where the vasculature of donor and recipient organs is different.

Also in FIGS. 35A and 35B, the contributing vessels separate, the fraction of the blood volume tapped or drawn off from each for joint flow through the confluence conduit evident, the relative proportion effluent into each of the outlet vessels relegated to self-apportionment. However, where there is reason to exercise control over the apportionment of outflow volume, the outlet connectors can be servovalves. At the inlet end, with the servovalve diversion chutes fully extended, the entire volume of each vessel is passed into the confluence conduit.

At the outlet end the servovalves can be used as trimmers to adjust inflow into each of the outlet vessels. In many cases, this degree of control will be unnecessary so that the expense of servovalves can be avoided. In FIGS. 35A thru 35F, the number of veins between donor and recipient organs are nonanomalous and equal, as this allows FIGS. 35D thru 35F to avoid the additional complexity of adding an artery or vein at either organ.

The benefits in the use of confluence conduits considerably exceeding those of just simplifying the accommodation of a mismatch between recipient and graft organs in the number of arterial and venous vessels, for visual consistency in FIGS. 35C and 35D, mismatching relatively less common with veins, it is the venous flow that has been shown. Another benefit responds to the current lack of prosthetic means for the durable and dependable repair of congenitally defective vasculature, especially of a large aneurysm or a severe coarctation of the thoracic aorta or an interrupted aortic arch where the prosthesis expands in step with growth.

Such eliminates the need for revision in a frail child, and therewith, the trauma of repeated operations, as well as the risk of sudden death due to a rupture, the rapid growth of a neonate notwithstanding. For pediatric patients congenitally impaired due to a connective tissue disorder and further debilitated by repeated operations, for example, such prostheses allow the elimination up to adolescence if not for life of the need for revision due to growth with adjacent areas of a congenital aneurysm, for example, having already been replaced to preclude its spread (see, for example, Erhart, P., Korfer, D., Dihlmann, S., Qiao, J. L., Hausser, I., and 6 others 2022. “Multiple Arterial Dissections and Connective Tissue Abnormalities,” Online, MDPI [Multidisciplinary Digital Publishing Institute] Journal of Clinical Medicine (Basel, Switzerland) 11(12): 3264; Wheeler, J. B., Ikonomidis, J. S., and Jones, J. A. 2021. “Connective Tissue Disorders and Cardiovascular Complications: The Indomitable Role of Transforming Growth Factor-beta Signaling,” Online, Advances in Experimental Medicine and Biology (New York, New York) 802:107-127; Takeda, N., Hara, H., Fujiwara, T., Kanaya, T., Maemura, S., and Komuro, I. 2018. “TGF-beta [Transforming Growth Factor-beta] Signaling-related Genes and Thoracic Aortic Aneurysms and Dissections,” Online, MDPI [Multidisciplinary Digital Publishing Institute] International Journal of Molecular Sciences (Basel, Switzerland) 19(7): 2125; Halper, J. (ed.) 2014. Progress in Heritable Soft Connective Tissue Diseases, volume 802 in the book series Advances in Experimental Medicine and Biology, Dordrecht, South Holland, Netherlands: Springer Verlag; additional references below in this section). No less important, should revision for any reason still become necessary, the patient will have had more time to grow and gain in strength.

Confluence conduits can have a different number of inputs or output lines at either end and can be reversed in direction as necessary. To match the number of vessels to be connected, input and output lines are not branched but rather separate and separately connected, thus affording specific access to measure pressures and apply adjustive measures if necessary, such as pressure relief in the form of a servovalve tap to another vessel or pressure increase through the use of a servochoke. This versatility makes confluence conduits no less advantageous in placing kidneys which require connection to an anomalous number of arteries and/or veins than connection to a different number of vessels by virtue of heterotopic placement.

Indeed, involving fewer vascular connections, less complex interrelations in intraparenchymal blood pressures than a heterotopically transplanted kidney positioned in the lower abdomen, and not positioned so distant from the orthotopic location that gravity further complicates matters, heterotopic placement of kidneys tends to be the norm in conventional kidney transplantation. While proximal heterotopic positioning of a second heart should preserve the correspondence in reciprocal cross circulatory vascular connections as in orthotopic placement, the versatility of connection made possible by ductus side-entry connectors, valves, and servochokes, for example, offer novel approaches for local connections that likely make it possible to overcome the deviations in intracardiac pressures that spell the ultimate failure of conventional local connections in the lower abdomen.

The placement of a vascular prosthesis along a major such as a great vessel to replace a severe defect thereof such as a pronounced coarctation should be considered if and only if a conventional surgical repair would not be likely to institute normal circulation and/or would be susceptible to breakdown that could result in dehiscence and hemorrhaging which if not stifled promptly would result in death. In a small child, there is the additional challenge that the prosthesis must expand in step with the growth of the child so that a conventional repair sets the stage for a reoperation. Abnormalities in blood flow are known to result in the degradation of the tissues and organs.

Unless a prosthetic segment inserted along a native vessel such as the thoracic, thoracoabdominal, or abdominal aorta expands and contracts in step with the pulse and blood pressure as does the tissue surrounding it, distinctions and discontinuity in coefficients of expansion between native and synthetic materials will interpose a perturbation in the pressure of blood flow-through to detract from the continuity of flow of the blood. Moreover, this consistency must persist despite growth. As the end-connectors used to secure a vascular prosthesis in alignment with the wider vessel, inline coupling jackets must not increase in prominence as points of impasse but rather expand in step with growth.

For placement in a child, confluence conduits and vascular prostheses are made of the same polypropylene or polyetheretherketone structure described below in section 2b(2)(f), entitled Expandable Structure to Accommodate Growth and Self-adjust in Size in Vascular Segment Prostheses, Lines, and Confluence Conduits Placed in Children. The expansion with growth this affords eliminates the need for repeated reentry and revision which imposes much trauma, and if complicated, can pose an increased risk for the formation of adhesions despite the use of a barrier agent or film such as, hyaluronic acid and polyethylene glycol/polylactic acid.

Uterine adhesions, adhesive bands of scar tissue that form on the uterine endometrium, often in response to injury, which lead to endometrial fibrosis and obstruction of the uterine cavity to complicate delivery, can be associated with intrauterine, or fetal, growth restriction (see, for example, Guo, Z., Wang, Y., Wen, X., Xu, X, and Yan, L. 2022. “Beta-Klotho [a subfamily Klotho enzyme encoded by the KL [Klotho] gene] Promotes the Development of Intrauterine Adhesions via the PI3K/AKT [phosphatidylinositol 3-kinase, catalytic, α-polypeptide/protein kinase B, an intracellular cell cycle regulating signaling pathway] Signaling Pathway,” Online, MDPI [Multidisciplinary Digital Publishing Institute] International Journal of Molecular Sciences (Basel, Switzerland) 23(19): 11294; Wenzhi, X., Xin, X., Ping, Z., Hanglin, W., and Xiaona, L. 2023. “A Meta-analysis of Obstetric and Neonatal Outcomes in Patients after Treatment of Hysteroscopic Adhesiolysis,” Online, Frontiers in Endocrinology (Lausanne, Switzerland). 14:1126740; Hooker, A. B., de Leeuw, R. A., Emanuel, M. H., Mijatovic, V., Brolmann, H. A. M., and Huirne, J. A. F. 2022. “The Link between Intrauterine Adhesions and Impaired Reproductive Performance: A Systematic Review of the Literature,” Online, BMC [BioMed Central] Pregnancy and Childbirth. (London, England) 22(1): 837; Hooker, A. B., Mansvelder, F. J, Elbers, R. G., and Frijmersum, Z. 2022. “Reproductive Outcomes in Women with Mild Intrauterine Adhesions; a Systematic Review and Meta-analysis,” Online, Journal of Maternal-Fetal and Neonatal Medicine (London, England) 35(25): 6933-6941).

After decades of study, the permanent suppression of adhesion forming remains to be perfected (see, for example, Flutur, I. M., Paduraru, D. N., Bolocan, A., Palcau, A. C., Ion, D., and Andronic, O. 2023. “Postsurgical Adhesions: Is There Any Prophylactic Strategy Really Working?,” Online, MDPI [Multidisciplinary Digital Publishing Institute] Journal of Clinical Medicine (Basel, Switzerland) 12(12): 3931; Fatehi Hassanabad, A., Zarzycki, A. N., Jeon K., Dundas, J. A., Vasanthan, V., Deniset, J. F., and Fedak, P. W. M. 2021. “Prevention of Post-operative Adhesions: A Comprehensive Review of Present and Emerging Strategies,” MDPI [Multidisciplinary Digital Publishing Institute] Biomolecules (Basel, Switzerland. 11(7): 1027 (also cited below in this section); Greene, A. K., Alwayn, I. P., Nose, V., Flynn, E., Sampson, D., and 3 others 2005. “Prevention of Intra-abdominal Adhesions Using the Antiangiogenic COX-2 inhibitor Celecoxib,” Online, Annals of Surgery (Philadelphia, Pennsylvania) 242(1): 140-146).

While some adhesions are congenital, significantly reducing trauma and eliminating ischemia, the procedures described here likely reduce if not eliminate the formation of postsurgical adhesions (see, for example, Nahirniak, P. and Tuma, F. 2022. “Adhesiolysis,” Online, Treasure Island, Florida: StatPearls Publishing Co.; Fatehi Hassanabad, A., Zarzycki, A. N., Jeon, K., Deniset, J. F., and Fedak, P. W. M. 2021. “Post-operative Adhesions: A Comprehensive Review of Mechanisms,” Online, MDPI [Multidisciplinary Digital Publishing Institute] Biomedicines (Basel, Switzerland) 9(8): 867 (also cited below in this section).

Adhesions must be excised, if possible, endoscopically, and unlike most other causes for intrauterine, oe fetal growth restriction, which relate to biochemical factors and processes, adhesions are elusive to treatment with the aid of an implanted automated disorder response system. Imaging provides information confirming and indicating the status of the condition but is not therapeutic, and without such a system, blood testing, as imaging, can only be obtained in the clinic.

Associated with frequent fetal mortality as well as morbidity, a means for immediate diagnosis and response to symptoms of fetal growth restriction would be of much benefit. Based upon a medical history of the mother, it is often possible to predict the likelihood of fetal growth restriction prior to gestation, making it possible to implant the prospective gravida with symptom-pertinent response system components before gestation commences.

Identifying conditions indicative of fetal growth restriction in advance contributes to the lead-time afforded for the specification of implanted response system sensors and medicinal drug tree response algorithm appurtenant thereof contributes to pregestational preparation (see, for example, Chew. L. C. and Verma, R. P. 2023. “Fetal Growth Restriction,” Online, Treasure Island, Florida: StatPearls Publishing Co.: Taeidi, E., Ranjbar, A., Montazeri, F., Mehrnoush. V., and Darsareh, F. 2023. “Machine Learning-based Approach to Predict Intrauterine Growth Restriction,” Online, Cureus (Palo Alto, California) 15(7): e41448).

The place of an implanted response system is to immediately detect and respond to by dispensing medication to counteract any or all of the large potential number of biochemical, physiological, and metabolic, or metabolomic, deviations from the normal associated with fetal growth restriction. Associated with fetal growth restriction, hypothermia, hypoglycemia, polycythemia, maternal and/or fetal malperfusion, and maternal gestational hypertension, for example, given the pertinent sensors, inflammation of the placenta, or villitis, can all be monitored and responded to in utero (Sharma, D., Farahbakhsh, N., Shastri, S., and Sharma, P. 2016. “Intrauterine Growth Restriction-Part 2,” Journal of Maternal-Fetal and Neonatal Medicine (London, England) 29(24): 4037-4048). Such data can be obtained prior to or during gestation, but only the implanted system can react in real time, at any time or location, so that if the gravida is an outpatient, the incident-pertinent data is immediately transmitted to the clinic.

Early onset fetal growth restriction is discovered during ultrasound examination of the gravida prior to 32 weeks into gestation, generally at about 6 months (see, for example, Pels. A., Beune, I. M., van Wassenaer-Leemhuis, A. G., Limpens, J., and Ganzevoort. W. 2020. “Early-onset Fetal Growth Restriction: A Systematic Review on Mortality and Morbidity,” Online, Acta Obstetricia et Gynecologica Scandinavica (Malden, Massachusetts) 99(2): 153-166; Gutaj. P. and Wender-Ozegowska, E. 2016. “Diagnosis and Management of IUGR in Pregnancy Complicated by Type 1 Diabetes Mellitus,” Online, Current Diabetes Reports (Philadelphia, Pennsylvania) 16(5): 39). A gravida whose medical history and known condition suggest a susceptibility to fetal growth restriction is implanted with suspect symptom-pertinent sensors and counteracting drug reservoirs upon confirmation of pregnancy (see, for example, Anderson, J. and Ghaffarian, K. R. 2023. “Early Pregnancy Diagnosis,” Online, Treasure Island, Florida: StatPearls Publishing Co.).

Exceptionally, sensors and small drug reservoirs other than those positioned in the gravida are implanted in the fetus and placenta once these become operable by temporary removal from the uterus. Otherwise, the fetus itself is shielded from invasive trauma. Accordingly, the placenta and fetus are implanted as soon as temporary removal from the uterus for ex utero intrapartum treatment becomes feasible, generally at about 16 weeks into gestation (see, for example, Chung, W. and Lim, C. 2021. “Intraoperative Management for Ex-utero Intrapartum Treatment: Focusing on the Fetus,” Online, Anesthesia and Pain Medicine (Seoul, South Korea) 16(4): 329-337; Sangaletti, M., Garzon, S., Raffaelli, R., D'Alessandro, R., Bosco, M., and 5 others 2021. “The Ex utero Intrapartum Treatment (EXIT) Procedure: Case Report of a Multidisciplinary Team Approach,” Online, Acta Biomedica (Parma, Italy) 92 (S1): e2021142; Bence, C. M. and Wagner, A. J. 2019. “Ex utero Intrapartum Treatment (EXIT) Procedures,” Seminars in Pediatric Surgery (Philadelphia, Pennsylvania) 28(4): 150820; Pucher, B., Szydlowski, J., Jonczyk-Potoczna, K., and Sroczynski, J. 2018. “The EXIT (Ex-utero Intrapartum Treatment) Procedure—from the Paediatric ENT Perspective,” Online, Acta Otorhinolaryngologica Italica (Pisa, Italy) 38(5): 480-484; Saxena, K. N. 2009. “Anaesthesia for Fetal Surgeries,” Online Indian Journal of Anaesthesia 53(5): 554-559; Kuczkowski, K. M. 2007. “Advances in Obstetric Anesthesia: Anesthesia for Fetal Intrapartum Operations on Placental Support” Journal of Anesthesia (Tokyo, Japan) 21(2): 243-251).

Data in addition to the medical history of the prospective mother and the genetics of the parents, such as aberrant maternal blood plasma has not only real-time system therapeutic but predictive value (see, for example, Alsharairi, N. A. and Li, L. 2023. “Gut Microbiota, Inflammation, and Probiotic Supplementation in Fetal Growth Restriction-A Comprehensive Review of Human and Animal Studies,” Online, MDPI [Multidisciplinary Digital Publishing Institute] Life (Basel, Switzerland) 13(12): 2239; Barak. O., Lovelace. T., Piekos, S., Chu, T., Cao, Z., and 8 others 2023. “Integrated Unbiased Multiomics Defines Disease-independent Placental Clusters in Common Obstetrical Syndromes,” Online, BioMed Central Medicine (London, England) 21(1): 349; Ho. S. Y., Yuliana, M. E., Chou, H. C., and Chen, C. M. 2023. “Intrauterine Growth Restriction Alters Kidney Metabolism at the End of Nephrogenesis,” Online, Nutrition and Metabolism (London, England) 20(1): 50; Hong. J. and Kumar, S. 2023. “Circulating Biomarkers Associated with Placental Dysfunction and Their Utility for Predicting Fetal Growth Restriction,” Online, Clinical Science (London, England) 137(8): 579-595; Huang, N., Chen, W., Jiang, H., Yang, J., Zhang, Y., and 6 others 2023. “Metabolic Dynamics and Prediction of sFGR and Adverse Fetal Outcomes: A Prospective Longitudinal Cohort Study, Online, BioMed Central Medicine (London, England). 21(1): 455; Starodubtseva, N. L., Tokareva, A. O., Volochaeva, M. V., Kononikhin, A. S., Brzhozovskiy, A. G., and 8 others 2023. “Quantitative Proteomics of Maternal Blood Plasma in Isolated Intrauterine Growth Restriction, Online, MDPI [Multidisciplinary Digital Publishing Institute] International Journal of Molecular Sciences (Basel, Switzerland, 24(23): 16832; Taeidi, E., Ranjbar, A., Montazeri, F., Mehmoush, V., and Darsareh, F. 2023, Op cit. above this section; Sufriyana, H., Salim, H. M., Muhammad, A. R., Wu, Y. W., and Su, E. C. 2022. “Blood Biomarkers Representing Maternal-Fetal Interface Tissues Used to Predict Early- and Late-onset Preeclampsia but Not COVID-19 Infection,” Online, Computational and Structural Biotechnology Journal (Amsterdam, North Holland, Netherlands) 20:4206-4224; Yao. M., Xiao, Y., Yang, Z., Ge. W., Liang. F., and 3 others 2022. “Identification of Biomarkers for Preeclampsia Based on Metabolomics,” Online, Clinical Epidemiology (Auckland, New Zealand) 14:337-360; Yao M, Yang Z, Rong X. Hu X. Yao N, and 4 others 2022. “The Exploration of Fetal Growth Restriction Based on Metabolomics: A Systematic Review,” Online, MDPI [Multidisciplinary Digital Publishing Institute] Metabolites (Basel, Switzerland. 12(9): 860; Binder, J., Kalafat, E., Palmrich, P., Pateisky, P., and Khalil, A. 2021. “Angiogenic Markers and Their Longitudinal Change for Predicting Adverse Outcomes in Pregnant Women with Chronic Hypertension,” American journal of obstetrics and gynecology (New York, New York) 225(3): 305.e1-305.e14; Austdal. M., Silva, G. B., Bowe, S., Thomsen, L. C. V., Tangeras. L. H., and 3 others 2019. “Metabolomics Identifies Placental Dysfunction and Confirms Flt-1 (FMS-Like Tyrosine Kinase Receptor 1) Biomarker Specificity,” Online, Hypertension (Hagerstown, Maryland) 74(5): 1136-1143; Rabindran, N F N and Gedam, D. S. 2016. “Predictors of Intrauterine Growth Restriction,” Online, Journal of Obstetrics and Gynecology 2(2): 1-2; Albu, A. R., Anca, A. F., Horhoianu, V. V., and Horhoianu, I. A. 2014. “Predictive Factors for Intrauterine Growth Restriction,” Online, Journal of Medicine and Life (Bucharest Romania) 7(2): 165- 171; Conde-Agudelo. A., Papageorghiou, A. T., Kennedy, S. H., and Villar, J. 2013. “Novel Biomarkers for Predicting Intrauterine Growth Restriction: A Systematic Review and Meta-analysis,” Online, British Journal of Obstetrics and Gynaecology (Oxford, England) 120(6): 681-694).

Based upon the inhibition of the beta-Klotho and the PI3K/AKT pathway during development of the brain by its inhibitor, the Phosphatase and TENsin [a cell membrane localized protein that links actin filaments to integrin receptors] homolog (PTEN) to limit the proliferation of cells, the latter should reduce the transformation of endometrial stromal fibroblasts into myofibroblasts to reduce the formation of adhesions, and in this way, also help to prevent cancer (Rafalski, V. A. and Brunet, A. 2011. “Energy Metabolism in Adult Neural Stem Cell Fate,” Progress in Neurobiology (Oxford, England) 93(2): 182-203)(see also, Benites, J., Valderrama, J. A., Contreras, A., Enriquez, C., Pino-Rios. R., Yañez, O., and Buc Calderon, P. 2023. “Discovery of New 2-Phenylamino-3-acyl-1,4-naphthoquinones as Inhibitors of Cancer Cells Proliferation: Searching for Intra-cellular Targets Playing a Role in Cancer Cells Survival,” Online, MDPI [Multidisciplinary Digital Publishing Institute] Molecules (Basel, Switzerland) 28(11): 4323; Lei, X. and Jiao, J. 2018. “UTX [ubiquitously transcribed tetratricopeptide repeat, X chromosome, or lysine-specific demethylase 6A, an H3K27me3 demethylase encoded by the KDM6A gene] Affects Neural Stem Cell Proliferation and Differentiation through PTEN [phosphatase and tensin homolog inhibitor of the PI3K/AKT pathway] Signaling,” Online, Stem Cell Reports (Cambridge, Massachusetts) 10(4): 1193-1207; Shi, Y., Paluch, B. E., Wang, X., and Jiang, X. 2012. “PTEN [phosphatidylinositol (3,4,5)-trisphosphate [PtdIns(3,4,5)P3] at a Glance,” Online, Journal of Cell Science (Cambridge, England) 125(Pt 20): 4687-4692; Li, J., Yen, C., Liaw, D., Podsypanina, K., Bose, S., and 12 others 1997. “PTEN, [Phosphatase and TENin homolog] a Putative Protein Tyrosine Phosphatase Gene Mutated in Human Brain, Breast, and Prostate Cancer,” Science 275(5308): 1943-1947).

A fully implanted automatic disorder response system can release substances known to promote propitious PTEN such as casein kinases and glycogen synthase kinase 3beta into the circulatory system on a scheduled basis in response to sensor feedback (see, for example, Gildiz, S. and Minko, T. 2023. “Nanotechnology-based Nucleic Acid Vaccines for Treatment of Ovarian Cancer, Online, Pharmaceutical Research (New York, New York) 40(1): 123-144; Langdon, C. G. 2023. “Nuclear PTEN's Functions in Suppressing Tumorigenesis: Implications for Rare Cancers, Online, MDPI [Multidisciplinary Digital Publishing Institute] Biomolecules (Basel, Switzerland) 13(2): 259; Liu, X., Li, X., Wu, G., Qi, P., Zhang, Y., and 17 others 2022. “Umbilical Cord Mesenchymal Stem Cell-derived Small Extracellular Vesicles Deliver miR-21 to Promote Corneal Epithelial Wound Healing through PTEN/PI3K/Akt Pathway, Online, Stem Cells International (New York, New York) 2022:1252557; Smith, I. N., Dawson, J. E., Krieger, J., Thacker, S., Bahar, I., and Eng. C. 2022. “Structural and Dynamic Effects of PTEN C-terminal Tail Phosphorylation,” Online, Journal of Chemical Information and Modeling (Washington, D. C.) 62(17): 4175-4190; Zhang, C., Ma, H. M., Dong. S. S., Zhang, N., He. P., and 6 others 2022. “Furin Extracellularly Cleaves Secreted PTENα/β to Generate C-terminal Fragment with a Tumor-suppressive Role,” Cell Death and Disease (London, England) 13(6): 532; Dai, H., Li, M., Yang, W., Sun, X., Wang. P., and 5 others 2020. “Resveratrol Inhibits the Malignant Progression of Hepatocellular Carcinoma via MARCH1 [membrane associated RING-CH 1]-induced Regulation of PTEN/AKT Signaling,” Online, Aging (Albany, New York) 12(12): 11717-11731; Masson, G. R. and Williams. R. L. 2020. “Structural Mechanisms of PTEN Regulation,” Online, Cold Spring Harbor Perspectives in Medicine (Woodbury, New York) 10(3): a036152; Shen, S. M., Zhang. C., Ge, M. K., Dong. S. S., Xia, L., and 9 others 2020. “Author Correction: PTENα and PTENβ Promote Carcinogenesis through WDR5 and H3K4 Trimethylation,” Nature Cell Biology (London, England) 22(1): 135; Boosani, C. S., Gunasekar, P., and Agrawal, D. K. 2019. “An Update on PTEN Modulators-A Patent Review,” Online, Expert Opinion on Therapeutic Patents (London, England) 29(11): 881-889; Shen, S. M. Zhang, C., Ge, M. K., Dong, S. S., Xia, L., and 9 others 2019. “PTENα and PTENβ Promote Carcinogenesis through WDR5 and H3K4 Trimethylation,” Nature Cell Biology (London, England) 21(11): 1436-1448; Taylor, J. and Abdel-Wahab, O. 2019. “PTEN Isoforms with Dual and Opposing Function,” Nature Cell Biology (London, England) 21(11): 1306-1308; Naderali, E., Khaki, A. A., Rad, J. S., Ali-Hemmati, A., Rahmati, M., and Charoudeh, H. N. 2018. “Regulation and Modulation of PTEN Activity,” Molecular Biology Reports (Dordrecht, South Holland, Netherlands) 45(6): 2869-2881; Boosani, C. S. and Agrawal, D. K. 2013. “PTEN Modulators: A Patent Review, Online, Expert Opinion on Therapeutic Patents (London, England) 23(5): 569-580; Singh, G. and Chan, A. M. 2011. “Post-translational Modifications of PTEN and Their Potential Therapeutic Implications,” Current cancer drug targets. (Boca Raton, Florida) 11(5): 536-547; Chagpar. R. B., Links, P. H., Pastor, M. C., Furber, L. A., Hawrysh, A. D., Chamberlain, M. D., and Anderson, D. H. 2010. “Direct Positive Regulation of PTEN by the p85 Subunit of Phosphatidylinositol 3-kinase,” Online, Proceedings of the National Academy of Sciences of the United States of America (Washington, D. C.) 107(12): 5471-5476; Al-Khouri, A. M., Ma, Y., Togo, S. H., Williams, S., and Mustelin, T. 2005. “Cooperative Phosphorylation of the Tumor Suppressor Phosphatase and Tensin Homologue (PTEN) by Casein Kinases and Glycogen Synthase Kinase 3beta,” Online, Journal of Biological Chemistry (Baltimore, Maryland) 280(42): 35195-35202; intrauterine growth restriction is also addressed below in sections 2a (1), entitled Congenital Defects of the Heart and Other Organs—the Importance of Prompt Intervention; and 2b (2)(c), entitled Vascular Segment Prostheses: Inline Coupling Jackets and Bridging, or Tie-lines.

Adhesiogenic, endoscopy is less so than is open surgery, can be further reduced, and the reduction in adhesiogenesis and avoidance of adhesions in reoperations is an advantage cited in favor of endoscopic surgery, especially when combined with an adhesion barrier (see, for example, De Wilde, R. L., Devassy, R., Broek, R P. G. T., Miller, C. E., Adlan, A., and 16 others 2022. “The Future of Adhesion Prophylaxis Trials in Abdominal Surgery: An Expert Global Consensus” Online, MDPI [Multidisciplinary Digital Publishing Institute] Journal of Clinical Medicine (Basel, Switzerland) 11(6): 1476; Krielen, P., Grutters, J. P. C., Strik, C., ten Broek, R. P. G., van Goor, H., and Stommei, M. W. J. 2019. “Cost-effectiveness of the Prevention of Adhesions and Adhesive Small Bowel Obstruction after Colorectal Surgery with Adhesion Barriers: A Modelling Study,” World Journal of Emergency Surgery 14:41; Yoshida, N., Naito, Y., Yasuda, R., Murakami, T. Ogiso, K., and 11 others 2017. “A Novel Lens Cleaner to Prevent Water Drop Adhesions during Colonoscopy and Esophagogastroduodenoscopy,” Endoscopy International Open 5(12): E1235-E1241. erratum 5(12): C6; Nakamura, T., Sato, T., Naito, M. Ogura, N. Yamanashi, T. and 5 others 2016. “Laparoscopic Surgery is Useful for Preventing Recurrence of Small Bowel Obstruction after Surgery for Postoperative Small Bowel Obstruction,” Surgical Laparoscopy, Endoscopy, and Percutaneous Techniques 26(1): e1-e4; Nappi, C., di Spiezio Sardo, A., Greco, E., Guida, M., Bettocchi, S., and Bifulco, G. 2007. “Prevention of Adhesions in Gynaecological Endoscopy,” Human Reproduction Update 13(4): 379-394).

Common features of the methods for transplantation and the placement of the devices and prostheses set forth herein, each an improvement in the treatment of potentially fatal disease, are that:
1. Their use avoids the need for circulatory arrest through the affected segment, which if protracted, will impose a period of anoxia that will result in ischemia-reperfusion injury.
2. Regional rather than general anesthesia in balanced adults eliminates the adverse cognitive sequelae which often linger in the elderly.
3. Taken together, numerous causes for mid- and postprocedural complications due to a cessation of perfusion and the complications associated with general anesthesia are avoided.
4. With circulatory arrest through the affected segment, anoxia, disembodied storage, and extensive incisions eliminated, trauma is minute compared to that of a conventional transplantation.
5. An automatic control system administers and records not only the procedure itself but provides follow-up diagnostics and treatment, and
6. The procedures are reduced in complexity to the extent that a general surgeon can effectuate repairs superior to those that currently call for a specialist at a large medical center.

Even if not stored in a chilled static or flowing perfusate but implanted immediately following harvesting, the trauma imposed on a graft organ is formidable, that imposed in a conventional heart transplant being the most formidable of all, and the consequences of this initial trauma are never fully dispelled but persist to limit the viability of the graft. At the start of harvesting, transection of the blood supply and drainage with a sudden stoppage in the supply of oxygen, glucose, and amino acids.

The extent to which this shock is reduced when the organ is implanted depends upon the time the organ has been left in a disembodied state and the degree to which the donor and the eventual recipient match immunologically. The immediate reaction to excision is an integrated stress response which under ordinary circumstances where the insult is not interminable would represent an emergency protective strategy but which persisting beyond a certain point, transitions into autolytic self-destruction.

The cytokine storm that ensues damages not only the graft organ but all the organs, glands, and tissues of the donor sustained on life support, degrading all of these as potential replacements. The ultimate importance of this injury at the outset is that it is never fully overcome; the administration of immune tolerance induction drugs notwithstanding, there is no future miracle drug that will recover the sliced-through hearts of donor and recipient to a normal condition, and the portion of the graft organ transplanted will never overcome the shock of placement in an alien milieu. Here the object is to initiate life support prior to brain death, administer therapeutic support as appropriate, and in so doing, minimize irreversible damage to the graft organs of the donor.

Immunosuppressives, anti-inflammatoires, and the like only postpone rejection, which interrupted would ensue immediately (see, for example, van Zanden, J. E., t' Hart, N. A. Ottens, P. J., Liu, B., Rebolledo, R. A., Erasmus, M. E., and Leuvenink, H. G. D. 2021. “Methylprednisolone Treatment in Brain Death-induced Lung Inflammation—A Dose Comparative Study in Rats,” Online, Frontiers in Pharmacology 12:587003; Jawitz, O. K., Raman, V., Barac, Y., Anand J., Patel, C. B., and 3 others 2020. “Impact of Donor Brain Death Duration on Outcomes following Heart Transplantation: A UNOS [United Network for Organ Sharing] Registry Analysis,” Journal of Thoracic and Cardiovascular Surgery 159(4): 1345-1353; Sally, M. B., Malinoski, D. J., Zaldivar, F. P., Le, T., Khoshnevis, M., and 3 others 2018. “A Porcine Model to Study the Effect of Brain Death on Kidney Genomic Responses,” Journal of Clinical and Translational Science 2(4): 208- 216; Hahnenkamp, K., Bohler, K., Wolters, H., Wiebe K., Schneider, D., and Schmidt, H. H. J. 2016. “Organ-protective Intensive Care in Organ Donors,” Deutsches Ärsteblatt 113(33-34): 552-558; Pakos-Zebrucka, K., Koryga, I., Mnich, K., Ljujic, M., Samali. A., and Gorman, A. M 2016. “The Integrated Stress Response,” European Molecular Biology Organization Reports 17(10): 1374-1395; Robolledo, R. A., Hoeksma, D., Hottenrott, C. M. V., Bodar, Y. J. L., Ottens. P. J., Wiersema-Buist, J., and Leuvenink, H. G. D. 2016. “Slow Induction of Brain Death Leads to Decreased Renal Function and Increased Hepatic Apoptosis in Rats,” Journal of Transplantational Medicine 14:141; Wang, M. and Kaufman, R. J. 2016. “Protein Misfolding in the Endoplasmic Reticulum as a Conduit to Human Disease,” Nature 529(7586): 326-335; Niboer, W. N., Schuurs. T. A., Damman, J., van Goor, H., Vaiyda, V. S., and 3 others 2009. “Kidney Injury Molecule-1 is an Early Noninvasive Indicator for Donor Brain death-induces Injury Prior to Kidney Transplantation,”.American Journal of Transplantation 9(8): 1752-1759; Danobeitia, J. S., Sperger, J. M., Hanson, M. S., Park, E. E., Chlebeck, P. J., and 5 others 2011. “Early Activation of the Inflammatory Response in the Liver of Brain-dead Non-human Primates,” Journal of Surgical Research 176(2): 639-648; van der Hoeven, J. A. B., ter Horst, G. J., Molema, G. de Vos, P., Girbes. A. R. J., and 5 others 2000. “Effects of Brain Death and Hemodynamic Status on Function and Immunologie Activation of the Potential Donor Liver in the Rat,” Annals of Surgery (Philadelphia, Pennsylvania) 232(6): 804-813; Nygaard, C. E., Townsend, R. N., and Diamond, D. L. 1990. “Organ Donor Management and Organ Outcome: A 6-year Review from a Level I Trauma Center,” Journal of Trauma (Baltimore, Maryland) 30(6): 728-732; Cooper, D. K. C., Novitzky, D., and Wicomb. W. N. 1989. “The Pathophysiological Effects of Brain Death on Potential Donor Organs, with Particular Reference to the Heart,” Annals of the Royal College of Surgeons of England 71(4): 261-266).

That replacement of the native by the graft organ materially reduces the shock of direct confrontation between innate and alien tissue allows a reduction in the need for immunosuppressive drugs, thus reducing the susceptibility of the patient to infection that could result in failure. It is not unreasonable to consider that the profound reduction in trauma provided by compound vascular bypass as opposed to conventional organ harvesting and transplantation will offset the increasing impairment of the patient during the wait for a transplant, and thus extend the time the patient will remain qualified.

More directly, it is irrefutable that the less traumatizing the replacement of an innate with a well-matched replacement organ, the less will be the adverse sequelae; shifting a replacement organ into place uneventfully cannot be exceeded for its freedom from trauma. Such a method reduces to the absolute least the impact on the graft organ and the recipient. The damage done following the use of a conventional procedure, ameliorative measures only slow down the process of degradation and rejection to stall the inevitable. For the very elderly likely to die before rejection, the worst consequence is spared; however, for pediatric transplant recipients—some born with unsurvivable cardiac malformities—this factor is crucial. In compound vascular bypass orthotopic heart transplantation, no anastomoses are needed to implant a heart unincised and intact.

Severely injured at the outset, the primary need to heal saps the resources both hearts need to adjust to their new condition, and that initial damage is never completely dispelled but rather persists to detract from the durability of the procedure, which usually ends with late-term cardiac allograft vasculopathy largely attributed to precisely the conditions that compound vascular bypass transplantation eliminates, to include the cessation of circulation in the donor, delay and harmful storage of the donor, or graft, organ, and allograft ischemia and reperfusion injury.

Moreover, while as next delineated, extended immunosuppression in support of an allograft can critically reduce the resistance of the recipient, the direct transfer of the donor heart into the recipient under the sterile conditions in an operating room minimizes the risk of infection (see, for example, Abbasi, M. A., Blake, A. M., Samari. R., Lee, D., Anderson, A. S., and 8 others 2022. “Multiparametric Cardiac Magnetic Resonance Imaging Detects Altered Myocardial Tissue and Function in Heart Transplantation Recipients Monitored for Cardiac Allograft Vasculopathy, Online, Journal of Cardiovascular Imaging 30(4): 263-275; Brilliand, B., Laplante, P., Thebault. P., Geoffroy, K., Brissette, M. J., and 6 others 2022. “MFG-E8 [milk fat globule epidermal growth factor 8] Reduces Aortic Intimal Proliferation in a Murine Model of Transplant Vasculopathy” Online, MDPI [Multidisciplinary Digital Publishing Institute] International Journal of Molecular Sciences (Basel, Switzerland) 23(8): 4094; Clemmensen, T. S., Jensen, N. M., and Eiskjaer, H. 2021. “Imaging of Cardiac Transplantation: An Overview,” Seminars in Nuclear Medicine 51(4): 335-348; Husain, N., Watanabe, K., Berhane, H., Gupta, A., Markl, M., Rigsby, C. K., and Robinson, J. D. 2021. “Multiparametric Cardiovascular Magnetic Resonance with Regadenoson Stress Perfusion is Safe following Pediatric Heart Transplantation and Identifies History of Rejection and Cardiac Allograft Vasculopathy,” Online, Journal of Cardiovascular Magnetic Resonance 23(1): 135; Shetty: M. and Chowdhury, Y. S. 2021. “Heart Transplantation Allograft Vasculopathy,” Online, Treasure Island, Florida: StatPearls Publishing Co.; Kummer, L., Zaradzki, M., Vijayan, V., Aril. R., Weigand, M. A., and 3 others 2020. “Vascular Signaling in Allogenic Solid Organ Transplantation—The Role of Endothelial Cells,” Online, Frontiers in Physiology 11:443; Cao, R.-N., Tang. L. Xia Z.-Y. and Xia, R. 2019. “Endothelial Glycocalyx as a Potential Therapeutic Target in Organ Injuries,” Chinese Medical Journal 132(08): 963-975); Lee, M. S., Tadwalkar, R. V., Fearon, W. F., Kirtane, A. J., Patel, A. J., and 3 others 2018. “Cardiac Allograft Vasculopathy: A Review,” Catheterization and Cardiovascular Interventions 92(7): E527-E536; Chih, S., Chong, A. Y., Mielniczuk, L. M., Bhatt, D. L., and Beanlands. R. S. 2016. “Allograft Vasculopathy: The Achilles' Heel of Heart Transplantation,” Journal of the American College of Cardiology 68(1): 80-91; Fenton, M., Simmonds, J., Shah, V., Brogan, P., Klein, N., Deanfield, J., and Burch, M. 2016. “Inflammatory Cytokines, endothelial Function, and Chronic Allograft Vasculopathy in Children: An Investigation of the Donor and Recipient Vasculature after Heart Transplantation,” American Journal of Transplantation 16(5): 1559-1568; Delgado, J. F., Reyne, A. G., de Dios, S., López-Medrano, F., Jurado, A., and 6 others 2015. “Influence of Cytomegalovirus Infection in the Development of Cardiac Allograft Vasculopathy after Heart Transplantation,” Journal of Heart and Lung Transplantation (New York, New York) 34(8): 1112-1119: Alphonsus, C. S., and Rodseth, R. N. 2014. “The Endothelial Glaycocalyx: A Review of the Vascular Barrier,” Anesthesia 69(7): 777-784; Hognestad, A., Endresen, K., Wergeland, R., Stokke, O., Geiran, O., and 4 others 2003. “Plasma C-reactive Protein as a Marker of Cardiac Allograft Vasculopathy in Heart Transplant Recipients,” Journal of the American College of Cardiology(3): 477-482; Eisenberg, M. S., Chen, H. J., Warshofsky, M. K., Sciacca, R. R., Wasserman, H. S., Schwartz, A., and Rabbani. L. E. 2000. “Elevated Levels of Plasma C-reactive Protein are Associated with Decreased Graft Survival in Cardiac Transplant Recipients,” Circulation 102(17): 2100-2104).

Where metered switched-organ transplantation seamlessly transfers the graft organ from the circulatory system of the donor into that of the recipient allowing an initial measure of gradually increased immune tolerance induction by means of gradually increased reciprocal cross-circulation, a fully implanted automatic prosthetic disorder response system can directly pipe-target chemotherapeutic, immunosuppressive, anti-inflammatory, and antimicrobial medication to the implant, sparing nontargeted tissue exposure with consequent complications. This factor of drug or agent isolation from unintended tissue and interaction with other drugs, for example, removes a central obstacle to dosing at a level most likely to prove efficacious.

The probability that the combination of metered compound vascular, or switched-organ transplantation and directly pipe-targeted rather than systemic, or indiscriminate, cardiovascular protective, and immunosuppressive follow-up which includes the spleen, lymph nodes, thymus, and bone marrow by an implanted prosthetic disorder response system which can immediately detect the appearance of a malignancy and treat it will reduce if not eliminate the propensity toward post-transplantation carcinoma is no less significant than is the probable avoidance of cardiac allograft vasculopathy and other manifestations of post-transplantation degradation of the graft organ whether due to virus-induced infection if not lymphoproliferative malignancy.

Solid organ transplantation followed by systemic immunosuppression to include the spleen, lymph nodes, thymus, and bone marrow can result in malignancy and immunosuppression-related infection in other parts of the body (Kanade, R., Kler, A., and Banga, A. 2022. “Non-pulmonary Complications after Lung Transplantation: Part I. Indian Journal of Thoracic and Cardiovascular Surgery 38 (Supplement 2) 280-289; Part II. 38 (Supplement 2): 290-299)(see also Cheng, F., Li, Q., Wang, J., Wang, Z., Zeng. F., and Zhang, Y. 2022. “Retrospective Analysis of the Risk Factors of Perioperative Bacterial Infection and Correlation with Clinical Prognosis in Kidney Transplant Recipients,” Online, Infection and Drug Resistance 5:2271-2286; Friman, T. K., Jaamaa-Holmberg, S., Aberg. F., Helantera. I., Halme. M., and 6 others 2022. “Cancer Risk and Mortality after Solid Organ Transplantation: A Population-based 30-year Cohort Study in Finland,” Online, International Journal of Cancer 150(11): 1779-1791; Gogna, S., Ramakrishna, K., and John, S. 2022. “Post Transplantation Cancer,” Online, Treasure Island, Florida: StatPearls Publishing Co.; Grenda. R., 2022. “Non-Hodgkin Lymphoma after Pediatric Kidney Transplantation,” Online, Pediatric Nephrology (Berlin, Germany) 37(8): 1759-1773; Hu, L., Wu, Y., Ju, F., Zhang, Y., and Wang, W., 2022. “Effects of Solid Organ Transplantation on the Risk of Developing Thyroid Cancer: A Systematic Review and Meta-analysis,” Online, Gland Surgery 11(4): 710-719; Kim. T., Yeo, H. J., Jang. J. H., Kim, D., Jeon, D., Kim, Y. S., and Cho, W. H. 2022. “Prognostic Impact of Preoperative Respiratory Colonization on Early-onset Pneumonia after Lung Transplantation,” Online, Journal of Thoracic Disease 14(6): 1900-1908; Kirov, H., Moschovas. A., Caldonazo, T., Schwan, I., Faerber, G., and 3 others 2022. “Diabetes is an Independent Risk Factor for Cancer after Heart and/or Lung Transplantation,” Online, MDPI [Multidisciplinary Digital Publishing Institute] Journal of Clinical Medicine (Basel, Switzerland) 11(14): 4127; Levi, S., Davidovits, M., Alfandari, H., Dagan, A., Borovitz, Y., and 3 others 2022. “EBV [Epstein-Barr virus], CMV [cytomegalovirus], and BKV [BK (initials of patient) Viral Infections in Pediatric Kidney Transplantation: Frequency, Risk Factors, Treatment, and Outcomes,” Pediatric Transplantation 26(3): e14199; Otto. W. R. and Green, A. 2022. “Antiviral Therapeutics in Pediatric Transplant Recipients,” Infectious Disease Clinics of North America 36(1): 125-146; Park, M. S. 2022. “Medical Complications of Lung Transplantation,” Online, Journal of Chest Surgery (Seoul, South Korea) 55(4): 338-356; Rodriguez-Peralvarez, M., Colmenaro. J., Gonzalez, A., Gastaca, M., Curell, A., and 14 others 2022. “Cumulative Exposure to Tacrolimus and Incidence of Cancer after Liver Transplantation,” Online, American Journal of Transplantation 22(6): 1671-1682; Shen, B., Cen, Z., Tan, M., Song. C., Wu, X., Wang, J., and Huang, M. 2022. “Current Status of Malignant Tumors after Organ Transplantation,” Online, BioMed Research International 2022:5852451; Shen, Y., Lian, D., Shi, K., Gao, Y., Hu X., and 3 others 2022. “Cancer Risk and Mutational Patterns following Organ Transplantation,” Online, Frontiers in Cell and Developmental Biology 10:956334; Sun, T., Liu, Y., Cai, Y., Zhai, T., Zhou, Y., and 3 others 2022. “A Paired Comparison of Plasma and Bronchoalveolar Lavage Fluid for Metagenomic Next-generation Sequencing in Critically Ill Patients with Suspected Severe Pneumonia,” Online, Infection and Drug Resistance 15:4369-4379; Thet, Z., Lam, A. K., Ng. S. K., Aung, S. Y. Han, T., and 5 others 2022. “An Integrated Skin Cancer Education Program in Renal Transplant Recipients and Patients with Glomerular Disease,” Online, BioMed Central Nephrology 23(1): 361; Zhao, D., Guo, L., Lian, D., Gu, Y., Yan, X., Hu. H., and Yuan, J. 2022. “Diagnostic Value and Clinical Application of mNGS [metagenomic next- generation sequencing] for Post-liver Transplantation Infection: A Cross-sectional Study with Case Reports,” Online, Frontiers in Microbiology 13:919363; Chen, J., Zhao, Y., Shang, Y., Lin, Z., Xu. G., and 6 others 2021. “The Clinical Significance of Simultaneous Detection of Pathogens from Bronchoalveolar Lavage Fluid and Blood Samples by Metagenomic Next-generation Sequencing in Patients with Severe Pneumonia,” Online, Journal of Medical Microbiology 70(1); McCort, M., Mackenzie, E., Pursell, K., and Pitrak, D. 2021. “Bacterial Infections in Lung Transplantation,” Online, Journal of Thoracic Disease 13(11): 6654-6672; Parajuli, S., Jorgenson, M., Meyers, R. O., Djamali, A., and Galipeau, J. 2021. “Role of Virus-specific T Cell Therapy for Cytomegalovirus and BK Infections in Kidney Transplant Recipients,” Online, Kidney 360 2(5): 905-915; Samanta, P. Clancy, C. J., and Nguyen, M. H. 2021. “Fungal Infections in Lung Transplantation,” Online, Journal of Thoracic Disease 13(11): 6695-6707; Sekulovski, M., Simonska, B., Peruhova, M., Kratev, B., Peshevska-Sekulovska, M., Spassov, L., and Velikova. T. 2021. “Factors Affecting Complications Development and Mortality after Single Lung Transplant,” Online, World Journal of Transplantation (Pleasanton, California) 11(8): 320-334; Yetmar Z. A., Lahr, B., Brumble, L., Gea Banacloche, J., Steidley, D. E., Kushwaha, S. and Beam, E. 2021. “Epidemiology Risk Factors, and Association of Antifungal Prophylaxis on Early Invasive Fungal Infection in Heart Transplant Recipients,” Transplant Infectious Disease 23(5): e13714; Hosseini-Moghaddam, S. M., Ouedraogo, A., Naylor, K. L., Bota S. E., Husain, S., Nash, D.<M., and Paterson, J. M. 2020. “Incidence and Outcomes of Invasive Fungal Infection among Solid Organ Transplant Recipients: A Population-based Cohort Study,” Transplant Infectious Disease 22(2): e13250; Huo, Z., Li, C., Xu. X., Ge. F., Wang. R., and 14 others 2020. “Cancer Risks in Solid Organ Transplant Recipients: Results from a Comprehensive Analysis of 72 Cohort Studies,” Online, Onco Immunology (Philadelphia, Pennsylvania) 9(1): 1848068; Martinez, O. M. 2020. “Biomarkers for PTLD [Post-transplant lymphoproliferative disease] Diagnosis and Therapies,” Pediatric Nephrology (Berlin, Germany) 35(7): 1173-1181; Straichman, O., and Ahya, V. N. 2020. “Malignancy after Lung Transplantation,” Online, Annals of Translational Medicine 8(6): 416; Alsaeed, Ml and Husain, S. 2019. “Infections in Heart and Lung Transplant Recipients,” Critical Care Clinics 35(1): 75-93; Aravinthan, A. D., Fateen, W., Doyle, A. C., Venkatahalapathy, S. V., Issachar, A., and 11 others 2019. “The Impact of Preexisting and Post-transplant Diabetes Mellitus on Outcomes following Liver Transplantation,” Online, Transplantation 103(12): 2523-2530; Guenette, A. and Husain, S. 2019. “Infectious Complications following Solid Organ Transplantation,” Online, Critical Care Clinics 35(1): 151-168; Jaamaa-Holmberg, S., Salmela, B., Lemstrom, K., Pukkala, E., and Lommi, J. 2019. “Cancer Incidence and Mortality after Heart Transplantation: A Population-based National Cohort Study,” Online, Acta Oncologica 58(6): 859-863; Rossi, A. P., and Klein, C. L. 2019. ‘Posttransplant Malignancy,” Surgical Clinics of North America 99(1): 49-64; Blazquez-Navarro, A., Dang-Heine, C., Wittenbrink, N., Bauer, C., Wolk, K., and 8 others 2018. “BKV] BK virus], CMV [cytomegalovirus], and EBV [Epstein-Barr virus] Interactions and Their Effect on Graft Function One Year Post-renal Transplantation: Results from a Large Multi-centre Study,” Online, EBioMedicine 34:113-121; Colombini, E., Guzzo, I., Morolli, F., Longo, G., Russo, C., and 8 others 2017. “Viral Load of EBV [Epstein-Barr virus] DNAemia is a Predictor of EBV-related Post- transplant Lymphoproliferative Disorder in Pediatric Renal Transplant Recipients,” Pediatric Nephrology (Berlin, Germany) 32(8): 1433-1442, erratum 32(10): 2001; Yanik, E. L., Smith, J. M., Shiels, M. S., Clarke, C. A., Lynch, C. F., and 4 others 2017. “Cancer Risk after Pediatric Solid Organ Transplantation,” Online, Pediatrics 139(5): e20163893; van Keer, J., Droogne, W., van Cleemput, J., Vörös, G. Rega, F., and 3 others 2016. “Cancer after Heart Transplantation: A 25-year Single-centre Perspective,” Transplantation Proceedings 48(6): 2172-2177; Martin-Gandul, C., Mueller, N. J., Pascual, M., and Manuel, O. 2015. “The Impact of Infection on Chronic Allograft Dysfunction and Allograft Survival after Solid Organ Transplantation,” Online, American Journal of Transplantation 15(12): 3024-3040; Nijland, M. L., Kersten, M. J., Pals, S. T., Bemelman, F. J., and ten Berge, I. J. 2015. “Epstein-Barr Virus-Positive Posttransplant Lymphoproliferative Disease after Solid Organ Transplantation: Pathogenesis, Clinical Manifestations, Diagnosis, and Management,” Online, Transplantation Direct 2(1): e48; Vajdic. C. M., and van Leeuwen, M. T. 2009. “Cancer Incidence and Risk Factors after Solid Organ Transplantation,” Online, International Journal of Cancer 125(8): 1747-1754; Morton, M., Coupes. B., Robert, S. A., Johnson, S. L., Klapper, P. E., Vallely, P. J., and Picton, M. L 2014. “Epstein-Barr Virus Infection in Adult Renal Transplant Recipients,” Online, American Journal of Transplantation 14(7): 1619-1629; Strecker, T., Rosch, J., Weyand. M., and Agaimy, A. 2013. “Frequency and Spectrum of Metachronous Malignancies in Heart Recipients: An 11-year Experience at a German Heart Center,” Online, International Journal of Clinical and Experimental Pathology 6(3): 411-420).

Both allograft vasculopathy and malignancy are prone to arise consequent either to immune rejection of an allogeneic organ or a repositioning of an autologous organ in a functionally distinct milieu which physiologically misaligned, subjects the transplanted tissue to chronic irritation. The implanted sensors of the prosthetic disorder response system can signal the appearance of conditions not originally contemplated for further diagnosis and treatment by extracorporeal means. That degradation can pertain to repositioned autologous tissue eliminates rejection on the basis of genetic incompatibility, pointing instead to functional incompatibility and possibly an inadequacy of oxygenation or improper innervation as the cause.

Autologous repositioning pertains to the harvesting of colon to replace tissue resected at partial esophagectomy, where both tissues are gastrointestinal, support peristalsis, and yet are even more intolerant of repositioning than is the common taking of a segment of the ilium to serve as a conduit for the voiding of urine, gut not adapted for use thus and without functional similarity to urothelium. Instances of the arising of malignancy in autologous transplantation are documented with references below in section 2b(2)(1), entitled Compound Vascular Bypass Transplantation-associated and Unassociated Anastomosis Bypasses.

That a transplanted lung can induce malignancy in extrapulmonary tissue may be attributable to residual ‘daughter’ cells of the original carcinoma or long-term systemic immunosuppression where the site of malignancy had been more susceptible to metaplastic degradation, possibly attributable to a greater need for oxygen which the primary pulmonary insufficiency precluded. Sensors and the type pathogens or conditions these detect are specified below in this section. That allogeneic tissue implanted in the anatomically correct position and even other tissue is prone to develop malignancy, cardiovascular disease, and/or infection is primarily due to rejection and premetastatic long-term nontargeted immunosuppression which the direct pipe-targeting of immunosuppressives should ameliorate. If and when metastatic, immunosuppressives are administered in a systemic background dose generally lower than that directly targeted at the lesion.

In the most traumatizing transplantation of a solid organ, that of a heart, having been sectioned to remove the ventricles of the donor, eventual rejection is at least in part attributable to the residual effects of trauma and associated complications to which the graft had been subjected at the outset (see, for example, Abudan A., Kidd, B., Hild, P., and Gupta, B. 2021. “Obstruction of the Inferior Vena Cava following Bicaval Orthotopic Heart Transplantation,” European Heart Journal-Case Reports 5(2)(ytab046; Abrams, B., Hoffman, J., Aftab, M., Evers, J., and Seres, T. 2019. “A Rare Case of Stenosis at the Inferior Vena Cava to Right Atrium Anastomosis, after Bicaval Orthotopic Heart Transplantation,” Seminars in Cardiothoracic and Vascular Anesthesia 23(4): 418-421; Chaney, M. A., Lowe, M. E., Minhaj, M. M., Santise, G., and Jacobsohn, E., 2019. “Inferior Vena Cava Stenosis after Bicaval Orthotopic Heart Transplantation,” Journal of Cardiothoracic and Vascular Anesthesia 33(9): 2561-2568; Jungschleger, J. G. M., Boldyrev S. Y., Kaleda, V. I., and Dark, J. H. 2018. “Standard Orthotopic Heart Transplantation,” Annals of Cardiothoracic Surgery 7(1): 169-171; Morgan, J. A. and Edwards, N. M. 2005. “Orthotopic Cardiac Transplantation: Comparison of Outcome Using Biatrial, Bicaval, and Total Techniques,” Journal of Cardiac Surgery 20(1): 102-106; Kitamura, S., Nakatani, T., Bando, K., Sasako, Y., Kobayashi, J. and Yagihara, T. 2001. “Modification of Bicaval Anastomosis Technique for Orthotopic Heart Transplantation,” Annals of Thoracic Surgery 72(4): 1405-1406; Blanche, C., Valenza, M., Czer, L. S., Barath, P., Admon, D., and others 1994. “Orthotopic Heart Transplantation with Bicaval and Pulmonary Venous Anastomoses,” Annals of Thoracic Surgery 58(5): 1505-1509; Blanche, C., Czer, L. S., Valenza, M. and Trento, A. 1994. “Alternative Technique for Orthotopic Heart Transplantation,” Annals of Thoracic Surgery 57(3): 765-767; Blanche, C., Valenza, M., Aleksic, I., Czer, L. S., and Trento, A. 1994. “Technical Considerations of a New Technique for Orthotopic Heart Transplantation. Total Excision of Recipient's Atria with Bicaval and Pulmonary Venous Anastomoses,” Journal of Cardiovascular Surgery (Turin, Italy) 35(4): 283-287; Sievers, H. H., Weyand, M., Kraatz, E. G., and Bernhard, A. 1991. “An Alternative Technique for Orthotopic Cardiac Transplantation, with Preservation of the Normal Anatomy of the Right Atrium,” Thoracic and Cardiovascular Surgeon 39(2): 70-72).

More broadly and especially with respect to a conventional heart transplant, the basic and major reduction in the trauma of harvesting, storage, anoxia, and the transplantation procedure itself-especially when the compound vascular bypass technique is performed using the metered switch procedure to accomplish a measure of immune tolerance induction during as well as periprocedurally to the transfer of the new heart from the circulatory system of the deceased donor having been kept on life support into the circulatory system of the recipient, means that the odds for a sound and favorable outcome are improved. More generally, because compound vascular bypass transplantation is nowhere near as traumatic as a conventional transplant, prospects can tolerate the procedure when considerably more impaired. This is so whether the greater impairment is due to a more advanced stage in the disease recommending transplantation such as heart failure for a heart transplant, due to another morbidity, or both.

The severity of conventional procedures restricts their application. That is, patients considered incapable of withstanding the procedure are disqualified without placement on the organ waiting list in the first place. Then, currently, approximately 7500 patients who qualified for an organ transplant die while awaiting an organ transplant in the United States alone (see, for example, Goldsteyn, B. H. H. and Verhagen, A. M. C. 2021. “Deceased by Default: Consent Systems and Organ Patient Morality,” Online, PLoS [Public Library of Science] One 16(3): e0247719; Roest, S., Dengler, S. E. K., van Suylen, V., van der Kaaij, N. P., Damman, K., and 5 others 2021. “Waiting List Mortality and the Potential of Donation after Circulatory Death Heart Transplantation in the Netherlands,” Netherlands Heart Journal 29(2): 88-97; DeRoos, L. J., Marrero, W. J., Tapper, E. B., Sonnenday, C. J., Lavieri, M. S., Hutton, D. W., and Parikh, N. D. 2019. “Estimated Association between Organ Availability and Presumed Consent in Solid Organ Transplant,” Online, Journal of the American Medical Associated Network Open 2(10): e1912431; Husen, P., Homung, J., Benko, T., Klin, C., Willuweit, K. and 5 others 2019. “Risk Factors for High Mortality on the Liver Transplant Waiting List in Times of Organ Shortage: A Single-center Analysis,” Annals of Transplantation 24:242-251; Israni, A. K., Zaun, D., Rosendale, J. D., Schaffthausen, C., Snyder, J. J., and Kasiske, B. L. 2019. “OPTN/SRTR [Organ Procurement and Transplantation Network/Scientific Registry Transplant Recipients] 2017 Annual Data Report: Deceased Organ Donation,” American Journal of Transplantation 19 (Supplement 2): 485-516; Lee, S., Yoo, K. D., An, J. N., Oh, Y. K., Lim, C. S., Kim, Y. S. and Lee. J. P. 2019. “Factors Affecting Mortality during the Waiting Time for Kidney Transplantation: A Nationwide Population-based Cohort Study Using the Korean Network for Organ Sharing (KONOS) Database,” Online, PLoS [Public Library of Science] One 14(4): e0212748).

Infrequently performed, the incisional trauma and ischemia/reperfusion injury of a conventional heterotopic heart transplant-which does not involve complete transections—is largely confined to the donor heart and anastomoses among the major vessels. However, the donor heart undergoes the trauma associated with harvesting, storage, and ischemia/reperfusion injury, so that overall trauma is less than it is in a conventional orthotopic heart transplant but still critically greater than it is with a compound vascular bypass double intact heart heterotopic transplant wherein perfusion of both hearts is uninterrupted and neither heart is incised (Mico-Carnero, M., Zaouali, M. A., Rojani-Alfonso, C., Maroto-Serrat, C., Ben Abdennebi, H., and Peralta, C. 2022. “A Potential Route to Reduce Ischemia/Reperfusion Injury in Organ Preservation,” Online, Cells 11(17): 2763; additional references are cited below in this section, in section 2a(3), entitled Remotely Procured Graft Organ Preservation, as well as in other sections.

A double heart heterotopic heart transplant should avoid the problems encountered with a conventional heterotopic transplant, to include right lung atelectasis and retention of the dysfunctional native left ventricle, which “ . . . may continue to enlarge, develop mural thrombi, and have serious arrhythmias,” (Copeland, J. and Copeland H. 2016. “Heterotopic Heart Transplantation: Technical Considerations,” Operative Techniques in Thoracic and Cardiovascular Surgery 21(3): 269-280).

Postprocedural valve dysfunction, for example, would not result, and without the multiple insults imposed by the conventional procedure, the eventual durability of the procedure should be greater (see, for example, Cockrell, H. C., O'Brien, R., Carter, K. T., Shaw, T. B., Baran, D. A., and 3 others 2021. “Better Together: A Reappraisal of Heterotopic Heart Transplantation,” Transplant International 34(11): 2184-2191; da Silva, L. F. and da Silva, J. P. 2021. “Heterotopic Heart Transplant History and Concepts Cannot be Neglected-Witnessing the History and Learning with Previous Practices,” Brazilian Journal of Cardiovascular Surgery 36(3): 436-438; Copeland, J. and Copeland H. 2016, Op cit., preceding paragraph; Bleasdale, R. A., Banner, N. R., Anyanwu, A. C., Mitchess, A. G., Khaghani, A., and Yacoub, M. H. 2002. “Determinants of Outcome after Heterotopic Heart Transplantation,” Journal of Heart and Lung Transplantation (New York, New York) 21(8): 867-873; Pham, S. M., Kormos, R. L., and Griffith, B. P. 1996. “Native Cardiectomy in a Heterotopic Heart Transplant Recipient,” Journal of Thoracic and Cardiovascular Surgery 112(4): 1109-1111; da Silva, J. P., Cascudo, M. M., Baumgratz, J. F., Vila, J. H., Wafae Filho, M., de Carvalho Neto, D. O., and da Fonseca, L. 1994. “Heterotopic Heart Transplantation: A Direct Pulmonary Artery Anastomosis Technique,” Journal of Thoracic and Cardiovascular Surgery 108(4): 795; Bolman, R. M 3d 1990. “Cardiac Transplantation: The Operative Technique,” Cardiovascular Clinics 20(2): 133-145; Novitszky, D., Cooper, D. K. C., and Barnard, C. N. 1983. “The Surgical Technique of Heterotopic Heart Transplantation,” Annals of Thoracic Surgery 36(4): 476-482; additional references are provided in section (b5) below, entitled Double Heart Metered Compound vascular bypass, or sudden switched-organ Transplantation).

In compound vascular bypass transplantation, two overriding factors support a better outcome than in the corresponding conventional transplant. These are that the procedure itself is fundamentally less traumatizing, and that the prosthetic disorder response system continuously monitors to counter any adverse incident. Working together, these two factors mean that the odds for a successful outcome are improved at every stage:

1. Before the operation and the implantation of the prosthetic disorder response system by allowing some additional degradation in the condition of the patient while waiting for a new heart that would ordinarily be disqualifying for receiving a heart transplant. When the response system had been implanted well ahead of the transplant, the patient should be in a better condition at the time of the transplant. During the period leading up to the transplant, the prosthetic disorder response system monitors and maintains the health of the prospect as well as applies immune tolerance-inducing medication.

2. During the operation by eliminating the extensive incisions into both recipient and donor hearts of which neither represents an intact new heart but instead a chimeric sliced-through and mutilated replacement assembly incorporating parts from both the inmate and the alien heart in immediate and extensive direct contact. The prosthetic disorder response system administers the procedure by controlling the servovalves that gradually transfer the graft organ from the circulatory system of the donor into the circulatory system of the recipient, releases immunosuppressive and anti-inflammatory drugs according to the measured need therefor and not by estimation, maintains vigilance to detect procedure-related adverse reactions or other incidents to immediately counter these, as well as continues to monitor and respond to the usual medical needs of the patient. A larger number of drugs needed midprocedurally are supplied from a supplementary drug delivery console plugged into the body surface port

3. After the operation by keeping the patient in as robust a condition as possible and reducing the degradation of the new heart that would otherwise inexorably progress to cardiac allograft vasculopathy and rejection.

As a result of avoiding this injury, the durability of a heart transplant performed using the metered compound vascular bypass technique described herein should materially exceed the ordinarily dozen or so years that follow a conventional heart transplant before rejection sets in. These capabilities make it possible to deal with multiple defects in a single operation taking a fraction of the time needed using conventional methods and achieve a result superior both in function and durability. Other benefits attributable to the critical reduction in trauma are that postprocedural healing times should be a minor fraction of those following conventional transplantation, and hearts that would ordinarily be incapable of tolerating a conventional heart transplant may qualify.

This is of special significance in conjunction with double heart transplantation as addressed below in the section 2b(1)(b)(b5) entitled Double Heart Metered Compound Vascular Bypass, or Switched-organ Transplantation [and so on], wherewith a patient unqualified to be placed on the waiting list can receive an o gan too imperfect to qualify. Because a double heart transplant requires the preparation of a site to position the second heart, it involves trauma of which the overall minimization to include other sources compensates for as best can be managed.

Genetic defects often pleiotropic, meaning the same gene or genes control the development of different organs and tissues, prenates and neonates can present with multiple malformities affecting different organs and tissues. A connective tissue disorder can result in venous as well as arterial aneurysms at birth. That multiple defects can be disposed of in a single operation which includes replacement of an irreparably defective organ with a good one and replacement of defective vasculature with a prosthesis, both placed without an interruption in perfusion and therefore eliminating ischemia/reperfusion injury, or the need for general anesthesia in a single operation, critically reduces the trauma and increases the odds for survival.

“Literature review revealed an association between double aortic arch and congenital heart diseases in approximately 20% of cases, most often tetralogy of Fallot [associated with] transposition of [the] great vessels [and/or] ventricular septal defects. Rarely there can be atresia of a segment of the aortic arch, which can be difficult to differentiate from other aortic arch anomalies associated with chromosomal abnormalities such as microdeletion of chromosome 22q11,” (Budziszewska, P., Kuka, D., Sodowski, M., Sodowski, K., Jeanty, P., and Skrzypulec, V, 2009. “Double Aortic Arch: Prenatal Case Report,” (in Polish with English abstract at Pubmed), Ginekologia Polska 80(9): 712-715).

No less than do these factors contribute to a reduction in mortality, a critical expansion in the number of surgeons able to administer the procedures adds increased accessibility to the factors favoring survival. A neonatal heart too malformed for the operator to initiate normal circulation or a severely interrupted aortic arch can each alone vitiate the life to ensue in quality and potential. However, the ability to replace a severely defective heart and segment if not the entire thoracic aorta, for example, in a single operation, without the need to interrupt the flow of blood and in rational adults, without general anesthesia, would be more likely than any alternative disposition to recover the patient for a normal life.

Moreover, where the need for postoperative medication is automatically met instantly despite a defective cognitive status of the patient, the burden of lifelong drug use and the threat of rejection are dispelled. Here a totally implanted drug release system administers pipeline-targeted drug delivery that eliminates systemic immunosuppression. Furthermore, organ transplantation and vessel replacement materially reduced in trauma, and simplified so that a general surgeon can undertake these procedures, prompt access is created, materially increasing the prospect for a successful outcome. Unless reparable to yield normal function, organs should be replaced as soon as possible, not just affected surgically to an extent that will no more than sustain a life low in quality.

### Prospective Reduction in Drug Dosing and Duration of Use

Referring to graft versus host disease, late-term cardiac allograft vasculopathy, and the inducement of malignancy that leads to graft failure, the avoidance of rejection and postoperative degenerative change in the graft organ made possible by metered compound bypass transplantation with immune tolerance induction should allow drugs conventionally used to counteract these sequelae to be much reduced, some perhaps eliminated. This is not to say that the initial shock of insertion into an alien milieu would allow the use of immunosuppressives to be reduced at the time of organ transfer, but rather that maintenance immunosuppression over the ensuing years during which late-term cardiac allograft vasculopathy and sometimes malignancy develops should allow the use of these potentially injurious drugs to be reduced.

Indispensable for graft survival, the use of these drugs nevertheless demands inordinate attention to their potentially ruinous effects when combined with other drugs. For example, combining statins with immunosuppressives, calcium channel blockers, antibiotics such as clarithromycin, azole antifungals such as itraconazole, amiodarone, gemfibrozil, virus protease inhibitors such as ritonavir, and several other drugs promotes myopathy and toxic lever disease. Nevertheless, “The clinical benefits of statin use after heart transplantation significantly exceed the risk of potential adverse effects, as demonstrated by numerous studies” (Szygula-Jurkiewicz, B., Szczurek, W., and Zembala, M. 2015. “The Role in Statins in Patients after Heart Transplantation,” Online, Kardiochirurgia i Torakochirurgia Polska (Polish Journal of Cardiothoracic Surgery). In medicine, balance can be critical.

In reducing the dosing of immunosuppressives, the method for seamlessly transferring a solid organ between individuals should also reduce if not eliminate the potentially severe adverse side effects of these drugs which can do as much if not more harm than good. By definition, immunosuppressives impair the immune system, risking bacterial, viral, and fungal infections anywhere in the body. By avoiding the dispersal of drugs throughout the circulatory system, the direct drug targeting made possible by the connectors and druglines of a fully implanted prosthetic disorder response system critically reduce if not eliminate serious systemic side effects such as the cytopenias.

Generally less prominent in solid organ transplantation, secondary corticosteroids such as dexamethasone and the glucocorticoid triamcinolone, and cytotoxins other than azathioprine and methotrexate, such as chlorambucil and mercaptopurine, for example, have been omitted. The potential side effects of immunosuppressants, anti-inflammatoires, and other drugs are too numerous to list in full. Accordingly, this list includes only the more important drugs. Material reduction in the dose levels of these drugs, even the prospect of eventually eliminating some, will represent a substantial improvement in solid organ transplantation using the metered compound bypass method.

Many drugs, to include steroids and statins, cause more severe adverse side effects when administered in larger doses and/or over a longer period of time. These drugs may be beneficial in increasing the strength of congenitally defective vascular tissue. When used to increase the strength of vascular tissue downstream from a tie-line type prosthesis used to replace an aortic arch that had been affected by a severe defect, a drugline inserted into a prosthesis accessory channel makes it possible to target such drugs in stronger doses to the site with considerably reduced dispersion throughout the circulatory system. Discretionary targeting of these drugs, which exert a direct topical effect upon the target tissue before or after passing through the liver, confines contact to the tissue that benefits from these, while sparing the patient from an increase in susceptibility to infection from any pathogen anywhere in the body. Drugs which depend upon processing in the liver are prepared in the post-liver passage form.

The adverse side effects of drugs used in transplantation and to treat more serious localized disease is listed in what follows for three major reasons:

1. The first is that adverse side effects not only limit systemic dosages, which not targeted but rather diluted throughout the circulatory system demand too high a dose for unintended and too low a dose for the intended tissue.

2. The second is that the adverse side effects associated with all drugs equate to the contraindications for their prescription which represent an expansion rather than reduction in troubling symptoms and distress, often representing a provocation of other diseases such as diabetes equivalent to an additional disease in itself as serious if not more so than the primary disease, requiring an additional drug or drugs, and an instigant of yet further, tertiary serious disease, which is avoided through use of the means for the directly targeted delivery of drugs delineated in U.S. Pat. Nos. 11,013,858, 11,389,171, and 11,759,186.

3. The third is that these adverse side effect-based contraindications can be eluded through the support action of a fully implanted automatic disorder response system programmed to change the drug or drugs in use as necessary. The system can then alternate between either of two drugs and switch to the other when adverse side effects appear before the patient experiences the side effects, or the system can move on to a third drug which it can rotate along with the other two. Such use of the implanted system is elaborated upon in the section to follow entitled Automatic Drug Alteration and Rotation.

Briefly, the implanted system uses implanted sensors to continuously monitor and respond to the condition through the targeted release of drugs or electrical stimulation devices previously established as having been effective. When the system detects incipient evidence of an adverse reaction due to the drug in use, it side-steps the adverse side effects that would ensue by switching or gradually transitioning to an alternative drug in accordance with its prescription-program. The potential for adverse symptoms associated with a given drug usually multifarious, practicality dictates that adverse symptoms warranting the placement of sensors be limited to those prominent and menacing.

Similarly, when the sensors detect analytes associated with an untreated condition, the controller replaces the second drug with another and monitors the continued production of the experienced as well as prospective analytes symptomatic of and diagnostic for the disorder. Elevated alanine aminotransferase, for example, is indicative of damage to the liver, and elevated aspartate aminotransferase additionally associated with damage to the pancreas, heart, or kidneys. If a third or more alternative drugs have not been programmed with reservoirs implanted from the outset, such a finding of the need for one or more additional drugs requires the additional reservoir or reservoirs to be implanted and the program to be updated.

### Interconnectivity of Implanted Drug Reservoirs and Targets

Means for switching from a drug detected as inducing an unacceptably adverse drug reaction to another drug are described in U.S. Pat. No. 11,759,186, entitled Ductus Side-entry Jackets and Prosthetic Disorder Response Systems, such means comprising turret switching mechanisms. Using these turret based means, any drug implanted drug reservoir can be switched from any pertinent target to any other.

### Gaining Access to the Neurocranial, to Include the Cerebral, Microvasculature

Direct surgical entry into the brain is limited to procedures that remove lesions such as tumors and aneurysms. Such procedures include transsphenoidal hypophysectomy (see, for example, Zubair, A., and M. Das J. 2022. “Transsphenoidal Hypophysectomy,” Online, Treasure Island, Florida: StatPearls Publishing Co.) to make possible resectioning of pituitary tumors and similar transnasal procedures to remove tumors and aneurysms. However, such external approaches are neither intended nor able to treat nonfocal or nonlesional disorders such as a diffuse infection or degenerative brain disease.

To accomplish this requires infusion of the encephalic microvasculature that supplies and drains the cells of the brain, and this requires the use of drugs or drugs, often with backup support through the use of mannitol or temperature which can make it possible to cross the blood brain barrier. Delivery and drainage through the neurocranial microvasculature can, however, be used to treat any disease condition of the brain to include a tumor or infection. Radiation has a role to play in such treatment as well, and the combined use of medication and irradiation has been in use for many years.

Several conditions, to include diabetes, multiple sclerosis, Parkinson's disease, Alzheimer disease, stroke, and seizures, disrupt the blood brain barrier, lessening if not eliminating the need for active disruption using means such as medicinal or irradiating (Dong, X. 2018. “Current Strategies for Brain Drug Delivery,” Online, Theranostics 8(6): 1481-1493). The permeability of the barrier also decreases with age, so that the elderly should not be prescribed antimuscarinic anticholinergics for an overactive bladder lest these pass into the brain to block muscarinic receptors resulting in cognitive impairment to include dementia (see, for example, Harnod, T., Yang, Y. C., Chiu, L.-T., Wang, J.-H., Lin, S.-Z., and Ding, D.-C. 2021. “Use of Antimuscarinics is Associated with an Increased Risk of Dementia: A Retrospective Population-based Case-control Study,” Online, (Nature Research) Scientific Reports 11:4827). However, these factors may expedite the delivery of drugs into the cerebral microvasculature.

Intravesical injections of botulinum toxin A have been shown to significantly reduce the severity of symptoms associated with overactive bladder and chronic bladder pain syndrome for decades (see, for example, Faure Walker, N., Macpherson, F., Tasleem, A., and Rampal. T. 2023. “Interventions to Improve Tolerability of Local Anesthetic Intradetrusor Botulinum Toxin Injections: A Systematic Review,” Online, Neurourology and Urodynanies (New York, New York) 42(1): 23-32; Jiang, Y. H., Jhang, J. F., and Kuo, H. C. 2022. “The Clinical Application of Intravesical Botulinum Toxin A Injection in Patients with Overactive Bladder and Interstitial Cystitis,” Online, Tau Chi Medical Journal (Mumbai (Bombay), India) 35(1): 31-37; Chen, J. L. and Kuo, H. C. 2020. “Clinical Application of Intravesical Botulinum Toxin Type A for Overactive Bladder and Interstitial Cystitis,” Online, Investigative and Clinical Urology (Seoul, South Korea) 61 (Supplement 1): S33-S42).

Referring to FIG. 30, the urinary assist device shown can serve as a backup to allow voiding when intravesical botulinum toxin A injections are not fully adequate or cease to work, such as increased post-void residual volume, which would necessitate self-catheterization and the eventual loss in efficacy, which appears to be inevitable at about nine months following the procedure in any event (see, for example, Okui, N. 2024. “A Discrete Mathematics Approach for Understanding Risk Factors in Overactive Bladder Treatment,” Online, Cureus (Palo Alto, California) 16(1): e53245; Ganguly A. Tyagi S. Chermansky C. Kanai A, Beckel J. and 6 others 2023. “Treating Lower Urinary Tract Symptoms in Older Adults: Intravesical Options,” Online, Drugs and Aging (Auckland, New Zealan) 40(3):241-261; Okui, N., Ikegami, T., Hashimoto, T., Kouno, Y., Nakano, K., and Okui, M. A. 2023. “Predictive Factors for High Post-void Residual Volume in Older Females After OnabotulinumA Treatment for Severe Overactive Bladder Using a Machine Learning Model,” Online, Cureus (Palo Alto, California) 15(7): e42668; Ou, Y. C., Kao, Y. L., Ho, Y. H., Wu. K. Y., and Kuo, H. C. 2023. “Intravesical Injection of Botulinum Toxin Type A in Patients with Refractory Overactive Bladder-Results between Young and Elderly Populations, and Factors Associated with Unfavorable Outcomes,” Online, MDPI [Multidisciplinary Digital Publishing Institute] Toxins (Basel, Switzerland) 15(2): 95; Jiang, Y. H., Jhang, J. F., and Kuo, H. C. 2022, Op cit. in the preceding paragraph; Hsiao, S. M., Lin, H. H., and Kuo, H. C. 2017. “Urodynamic Prognostic Factors for Large Post-void Residual Urine Volume after Intravesical Injection of OnabotulinumtoxinA for Overactive Bladder,” Online, Scientific Reports (Nature Scientific Reports) 7:43753; Kao, Y. L., Ou. Y. C., and Kuo, H. C. 2022. “Bladder Dysfunction in Older Adults: The Botulinum Toxin Option,” Drugs and Aging (Auckland, New Zealand) 39(6): 401-416; Jiang, Y. H., Ong. H. L., and Kuo, H. C. 2017. “Predictive Factors of Adverse Events after Intravesical Suburothelial Onabotulinumtox Injections for Overactive Bladder Syndrome—A Real-life Practice of 290 Cases in a Single Center,” Neurourology and Urodynamics (New York, New York) 36(1): 142-147; Kuo, H. C., Liao. C. H., and Chung, S. D. 2010. “Adverse Events of Intravesical Botulinum Toxin A Injections for Idiopathic Detrusor Overactivity: Risk Factors and Influence on Treatment Outcome,” European Urology (Amsterdam, North Holland, Netherlands) 58(6): 919-926).

The blood brain barrier becomes more permeable with age and therefore more susceptible to the cognitive harm caused by anticholinergic drugs such a diphenhydramine, for example, long suspected to promote cognitive decline. Also, as one ages, drug clearance through the liver and kidneys slows so that drugs linger in the blood stream longer, and with age, ailments and therewith the use of drugs increases raising the odds for adverse drug-drug and drug-food interactions. Furthermore, the increase in fat and sarcopenia changes the bodily distribution of drugs in these tissues, and therewith, the distribution, clearance, or more precisely expressed, the pharmacodynamics, and pharmacokinetics of drug processing by the body.

Different methods have been devised to cross the blood brain barrier, those based on the use of nanoparticles usually compatible with and adaptable for incorporation into a fully implanted prosthetic disorder response system when properly shielded as indicated albeit in relation to a different process in FIG. 14C. “Inorganic nanomaterials have been widely used in biomedical applications. However, since they lack the property of biodegradability, most applications aim the tissue bioimaging for diagnostics. Nonetheless, several inorganic nanoparticles, including gold and silica nanoparticles, and carbon nanotubes have been used to deliver specific drugs across the blood-brain barrier,” (Fernandez, M., Nigro, M., Travagli, A., Pasquini. S., Vincenzi, F., and 4 others 2023. “Strategies for Drug Delivery into the Brain: A Review on Adenosine Receptors Modulation for Central Nervous System Diseases Therapy,” Online, MDPI [Multidisciplinary Digital Publishing Institute] Pharmaceutics (Basel, Switzerland) 15(10): 2441: Teleanu, D. M., Chircov. C., Grumezescu, A M., Volceanov. A., and Teleanu, R. I. 2018. “Blood-Brain Delivery Methods Using Nanotechnology,” Online, MDPI [Multidisciplinary Digital Publishing Institute] Pharmaceuticals (Basel, Switzerland) 10(4): 269). (see also, for example, Asensio Ruiz M A, Alonso Garcia Á, Bravo-Ferrer Moreno M L, Cebreiros-López I, and 3 others 2023. “In Vitro Hemocompatibility and Genotoxicity Evaluation of Dual-labeled [99mTe] Te-FITC-Silk Fibroin Nanoparticles for Biomedical Applications,” Online, MDPI [Multidisciplinary Digital Publishing Institute] Pharmaceuticals (Basel, Switzerland)(2): 248; Binelli L, Dini V. Amatori S. Scotognella T. Giordano A. and 7 others 2023. “Gold Nanorods as Radiopharmaceutical Carriers: Preparation and Preliminary Radiobiological In Vitro Tests,” Online, MDPI [Multidisciplinary Digital Publishing Institute] Nanomaterials (Basel, Switzerland) 13(13): 1898; Nayab, D. E., Din, F. U., Ali, H., Kausar, W. A, Urooj. S., and 4 others 2023. Nano Biomaterials Based Strategies for Enhanced Brain Targeting in the Treatment of Neurodegenerative Diseases: An Up-to-date Perspective,” Online, BioMed Central Journal of nanobiotechnology (London, England) 21(1): 477; Alajangi, H. K., Kaur, M., Sharma, A. Rana, S. Thakur, S. and 5 others 2022. “Blood-brain Barrier: Emerging Trends on Transport Models and New-age Strategies for Therapeutics Intervention against Neurological Disorders,” Online, Molecular Brain 15:49; El Kheir, W., Marcos, B., Virgilio, N., Paquette, B. Faucheux, N., and Lauzon, M. A. 2022. “Drug Delivery Systems in the Development of Novel Strategies for Glioblastoma Treatment” Online, ISRN [Improvement Science Research Network] Pharmaceutics 14(6): 1189; Pijeira, M. S. O., Viltres, H., Kozempel, J., Sakmar, M., Vlk, M., and 8 others 2022. “Radiolabeled Nanomaterials for Biomedical Applications: Radiopharmacy in the Era of Nanotechnology,” Online, EJNMMI [European Journal of Nuclear Medicine and Molecular Imaging] Radiopharmacy and Chemistry (London, England) 7(1): 8; Song, J., Lu, C., Leszek, J., and Zhang, J. 2021. “Design and Development of Nanomaterial-based Drug Carriers to Overcome the Blood-brain Barrier by Using Different Transport Mechanisms,” Online, MDPI [Multidisciplinary Digital Publishing Institute] International Journal of Molecular Sciences (Basel, Switzerland) 22(18): 10118; Ding. S., Khan, A. I., Cai, X., Song. Y., Lyu, Z., and 3 others 2020. “Overcoming the Blood-brain Barrier Transport: Advances in Nanoparticle-based Drug Delivery Strategies,” Online, Materials Today (Kidlington, England): Xie, J., Shen, Z., Anraku, Y., Kataoka, K., and Chen, X. 2019. “Nanomaterial-based Blood-brain-barrier (BBB) Crossing Strategies,” Online, Biomaterials 224:119491).

If either or both the treatment of a vasculitis or the effect on different structures in the brain results in one or more adverse side effects, the system when ordered by the patient or in response to analytes associated with the symptom or symptoms is programmed to automatically alternate the simvastatin and prednisone with one another or replace these with similar drugs that will cross the blood brain barrier unassisted or by means such as:

1. Formulation as a nanoparticulate (see, for example, Bentivoglio. V., Nayak, P., Varani, M., Lauri, C., and Signore, A. 2023. “Methods for Radiolabeling Nanoparticles (Part 3): Therapeutic Use,” Online, MDPI [Multidisciplinary Digital Publishing Institute] Biomolecules (Basel, Switzerland) 13(8): 1241; Gidwani, M and Singh, A. V. 2014. “Nanoparticle Enabled Drug Delivery across the Blood Brain Barrier: in Vivo and in Vitro Models, Opportunities, and Challenges,” Current Pharmaceutical Biotechnology 14(14): 1201-1212).

2 When exosome delivery encapsulated (Liu X., Xia, T., Fang, Y., Zuo. H., Dong, X., Xu. P., and Ouyang, J. 2022. “Overcoming the Blood-brain Barrier by Using a Multistage Exosome Delivery System to Inhibit Central Nervous System Lymphoma,” Nanomedicine: Nanotechnology, Biology, and Medicine 41:102523); or will do so when

3. The blood brain barrier is disrupted using one of the means specified next.

Of these, disruption poses greater risks, and especially in an implanted system, best avoided. If necessary, however, the conservative application thereof should avoid the risks. As to the various means for disruption, the object to automatically release medication in an ambulatory patient having no awareness of the process limits blood brain barrier disruption to that method which will not require returning the patient to the clinic. This favors concurrent intra-arterial mannitol cerebral infusion by release into the internal carotids through side-entry jackets but not focused ultrasound, laser interstitial thermotherapy, or non—thermal irreversible electroporation. Of these, the use of mannitol is the least invasive, adaptable for use by the fully implanted automatic prosthetic disorder response system contemplated, and can be considerably more conservative in dose level than when used to fight a glioblastoma.

These factors should minimize if not eliminate the risks of “altered glucose, the passage of plasma proteins across the barrier, and microembolism in the cerebral vasculature” To avoid the risks of stroke and seizure, hyperosmotic disruption in an ambulatory patient must initiate the release of mannitol in the least amount effective well in advance of the use of the drug to counter the vasculitis (see, for example, Rodruguez, A., Tatter, S. B., and Debinski, W. 2015. “Neurosurgical Techniques for Disruption of the Blood-brain Barrier for Glioblastoma Treatment,” Online, ISRN [International Scholarly Research Network] Pharmaceutics 7(3): 175-187; Woodworth, G. F., Dunn, G. P., Nance, E. A., Hanes, J., and Brem, H. 2014. “Emerging Insights into Barriers to Effective Brain Tumor Therapeutics,” Online, Frontiers in Oncology 4:126).

### Automatic Drug Switching

In the treatment of Cogan syndrome (Salman, E. J. and Tripathy, K. 2022. “Cogan Syndrome,” Online, Treasure Island, Florida: StatPearls Publishing Co.) in the absence of a comorbidity or old age as would disrupt the barrier without the need for special measures, two drugs to counteract the vasculitis are simvastatin and prednisone. Both will suppress the underlying vasculitis but the steroid is more likely to affect the different tissues in the brain differently (see, for example, Joels, M. 2018. “Corticosteroids and the Brain,” Online, Journal of Endocrinology 238(3): R121- R130. Merely to gain access to the tissue of the brain already suggests an important role for a system of drug administration that can detect the emergence of adverse side effects, automatically switch to another drug, wait for signs of an adverse side effect and return to the original or a third drug, and so on.

Automatic drug switching responsive to sensor feedback to the master control microprocessor can be sudden or consist of a gradual transition from the offending to the replacement drug. The latter compensates for adverse reactions induced by the sudden withdrawal of certain drugs, such as the platelet blocker clopidogrel or ticagrelor, for example, which abruptly stopped, can actually result in an increased tendency to form clot. The total volume of the platelet blocker, which is directly pipe-targeted to the affected site is far too small to induce thrombocytopenia.

For example, simvastatin, which lipophilic does not require disruption of the blood brain barrier, can be replaced by a nonsteroidal anti-inflammatory or another lipophilic anti-inflammatory statin such as atorvastatin, and prednisone can be substituted for with dexamethasone, which to cross the blood brain barrier may require an adjuvant or multistage exosome delivery system (Banks, W. A. 2012. “Brain Meets Body: The Blood-brain Barrier as an Endocrine Interface,” Online, Endocrinology 153(9): 4111-4119), or a different class of drug such as high do se (1 to 3 grams per milliliter) methotrexate or hemoglobin-coenzyme A reductase inhibitor (Pierzchlinska, A., Drozdzik, M., and Bialecka, M. 2021. “Possible Role for HMG-COA Reductase Inhibitors and Its Association with HMGCR [the HMG-COA reductase inhibitor gene] Genetic Variation in Parkinson's Disease,” Online, MDPI [Multidisciplinary Digital Publishing Institute] International Journal of Molecular Sciences (Basel, Switzerland), 22(22): 12198; Zakout, S. and Mohamad, S. 2021. “Case Report of Cogan Syndrome in a Child with Audiovestibular Dysfunction and Ocular Inflammation,” Online, Rheumatology Advances in Practice 5 (Supplement 1): rkab067,029; Bazan, F., Dobi, B., Royer, E., Curtit, L., Mansi, N., and 6 others 2019. “Systemic High-dose Intravenous Methotrexate in Patients with Central Nervous System Metastatic Breast Cancer,” Online, BioMed Central Cancer 19:1029; Saeedi Saravi, S. S., Saeedi Saravi, S. S., Arefidoust, A., and Dehpour, R. 2017. “The Beneficial Effects of HMG-COA Reductase Inhibitors in the Processes of Neurodegeneration,” Metabolic Brain Disease 32(4): 949-965; Prodduturi, P., and Bierman, P. J. 2012. “Current and Emerging Pharmacotherapies for Primary CNS Lymphoma,” Online, Clinical Medicine Insights: Oncology 6:219-231; Angelov, L., Doolittle, N. D., Kraemer, D. F., Siegal, T., Bamett, G. H., and 13 others 2009. “Blood-brain Barrier Disruption and Intra-arterial Methotrexate-based Therapy for Newly Diagnosed Primary CNS Lymphoma: A Multi-institutional Experience,” Online, Journal of Clinical Oncology 27(21): 3503-3509; Orsoni, J. G., Zavota, L., Vincenti, V., Pellistri, I., and Rama, P. 2004. “Cogan Syndrome in Children: Early Diagnosis and Treatment is Critical to Prognosis,” American Journal of Ophthalmology 137(4): 757-758; Ndiaye, I. C., Rassi, S., and Wiener-Vacher, S. R. 2002. “Cochleovestibular Impairment in Pediatric Cogan's Syndrome,” Pediatrics 109(2): E38), Mycophenolate which hydrophilic, also has reduced ability to cross the blood brain barrier (Ebrahimi, F., Koch, M., Pieroh, P., Ghadvan, C., Hobusch, C., Bechmann, I, and Dehghani, F. 2012. “Time Dependent Neuroprotection of Mycophenolate Mofetil: Effects on Temporal Dynamics in Glial Proliferation, Apoptosis, and Scar Formation,” Online, Journal of Neuroinflammation 9:89; rituximab, which like methotrexate, may achieve passage through disruption of the blood brain barrier (Calimeri, T., Marcucci, F., and Corti, A. 2021. “Overcoming the Blood-brain Barrier in Primary Central Nervous System Lymphoma: A Review on New Strategies to Solve the Problem,” Online, Annals of Lymphoma 5:20; Batchelor, T. T., Grossman, S. A., Mikkelsen, T., Ye, X., Desideri, S. and Lesser, G. J. 2011. “Rituximab Monotherapy for Patients with Recurrent Primary CNS Lymphoma,” Online, Neurology 76(10929-930; Wood, W. G., Eckert, G. P., Igbavboa, U., and Müller, W. E. 2010. “Statins and Neuroprotection: A Prescription to Move the Field Forward,” Online, Annals of the New York Academy of Sciences (New York, New York) 1199:69-76).

At 1 to 2 percent serum concentrations, leflunomide/terileflunomide can be prepared to cross the blood brain barrier (Gottle, P., Manousi, A., Kremer, D., Reiche, L., Hartung, H.-P., and Kury, P. 2018.” Online, Journal of Neuroinflammation 15:76), and similar properties pertain to azathioprine, or mercaptopurine as well as other drugs. When gradually transitioned into the drug delivery line so that relative proportions of either drug are monitored by the system, a ratio of the two recognized as that most efficacious is identified and recorded for future use.

### Prepositioning Components for Extracorporeal Remote Reprogramming

For complications predictable and chronic where the need for immediate medical support will be significant, the need for reentry is avoided by prepositioning disorder response system components to include unobtrusive sensors and druglines at the outset when the primary invasive procedure is undertaken. This will allow immediate response to predictable complications associated with any condition or combination thereof. Sensors are addressed below in this section.

Connected to the system master control microprocessor, tiny unobtrusive multianalyte/multimetabolite sensors existing and yet to be developed prepositioned along the vascular tree and digestive tract will detect analytes such as metabolites and nonmetabolic enzymes associated with any metabolic or neurodevelopmental disorders related to defects in complex lipid metabolism, amino acid synthesis, fatty acid synthesis, and inborn errors of metabolism. Suitable connectors for connecting implanted drug reservoirs to the circulatory system and the digestive tract are described in U.S. Pat. No. 11,389,171, entitled Integrated System for the Infixion and Retrieval of Implants, and U.S. Pat. No. 11,759,186, entitled Ductus Side-entry Jackets and Prosthetic Disorder Response Systems, as well as the valves and servovalves described herein

Developmental disorders and congenital defects demand placement as early in development as possible, continuous surveillance, and programmed immediate medicinal response that does away with the delays in going to the emergency room, or obtaining a medical appointment, then obtaining the prescription. The need to avoid reentry requires that the disorder control system microprocessor be remotely reprogrammable to respond to any complicating eventuality known to be more likely. The list of disorders to which this applies involves a potential infinitude of conditions and combinations thereof. However, the ability to meet the number of possibilities will be attainable because the condition or conditions will have been identified and the means for response to these determined in advance.

Most such conditions complex, a pertinent example will cite the simplest and most common of complications. Where the likelihood that drugs which cause intractable constipation will be needed, sensors should be prepositioned at points along the digestive tract to modulate the rate of peristalsis and the release of digestive substances to include hydrochloric acid, bile, as well as digestive enzymes. Multitudinous and frequently needed in the treatment of almost any chronic condition, drugs that induce constipation do so by suppressing motility of the stomach and peristalsis father along the tract. The detention in the absorption of medication and the increase in premature absorption of fluid this causes leads to constipation.

Not only do senility and Parkinson's disease cause constipation but the drugs used to treat the condition only intensity it. Such drugs numerous, a partial list includes opiates, opioids, analgesics, antipyretics, antithrombotics, angiotensin converting enzyme inhibitors, mineral supplements to include calcium and iron, antacids, nonsteroidal anti-inflammatoires, tricyclic antidepressants, antipsychotics, antispasmodics, sympathomimetics, antihistamines, and anticholinergic agents used to treat overactive bladder and urinary incontinence such as trospium chloride (see, for example, Diaz; S., Bittar; K., Hashmi, M. F., and Mendez, M. D. 2023. “Constipation,” Online, Treasure Island, Florida: StatPearls Publishing Company; Sizar. O., Genova, R., and Gupta, M. 2023. “Opioid-induced Constipation,” Online, Treasure Island, Florida: StatPearls Publishing Company, Ueki, T. and Nakashima, M. 2019. “Relationship between Constipation and Medication,” Online, Journal of University of Occupational and Environmental Health (Sangyo ika Daigaku Zasshi, Kitakyushu, Japan) 41(2): 145-151).

This simple example once realized will fundamentally improve the quality of life for the great number of persons so affected.

### Targeting the Cranial Microvasculature

The targeting of drugs does not mean that when appropriate, the concurrent use of a systemic dose is disallowed, albeit subject to the motive to reduce the systemic dose to the minimum effective. Drugs can induce adverse side effects in the form of pathology apart from and in addition to the primary combination of morbidities. Azathioprine, for example, can cause tremor in patients with Crohn's disease or following organ transplantation, pneumonitis in inflammatory bowel disease patients and those having undergone a kidney transplant, cardiogenic shock and Sweet's Syndrome in microscopic polyangiitis, and shock in undifferentiated erosive oligoarthritis.

In a suspect autoimmune vasculitis, Cogan syndrome, cited as exemplary, corticosteroid anti-inflammatoires may not be prescribable in the actual dose required to most if not completely suppress the symptoms without placing the patient at risk for psychosis, moon facies, convulsions, elevated blood pressure, and other serious adverse side effects. Cited as rare, this may be the result of frequent misdiagnosis as idiopathic keratitis or idiopathic hearing loss (Salman, E. J. and Tripathy, K. 2022. “Cogans Syndrome,” Online, Treasure Island, Florida: StatPearls Publishing Co.); (see also, for example, Hain, T. C. 2022. “Cogan's Syndrome,” Online at Dizziness-and-Balance.com; Mohseni, M. M., 2022. “Acute Limb Ischemia in Cogan Syndrome,” Online, American Journal of Case Reports 23: e935929-1-e935929-4; Salamat, A. and Strieth, S.2022. “Atypisches Cogan-syndrom als Differenzialdiagnose Eines Horsturzes,” [Atypical Cogan Syndrome as a Differential Diagnosis of Sudden Sensorineural Hearing Loss,” (in German with English abstract at Pubmed) HNO [Hals-Nasen-Ohrenheilkunde/Larynx-Nose-Ear Medicine] 70(5): 405-414 [the assertion that the disease was first described in 1945 is mistaken; Morgan and Baumgartner described it in 1934—see, for example, Iliescu, D. A. et al., 2015 cited below; Ribeiro, R., Serodio, J. F., Amaral, M. C., Duarte, J. A., Durao. C., Mendes, N., and Delgado Alves, J. 2021. “Sensorineural Hearing Loss and Systemic Autoimmune Disease: The Experience of a Systemic Immune-mediated Diseases Unit,” Online, Cureus (Palo Alto, California) 13(3): e14075; Venhoff, N., Thiel, J., Schramm, M. A., Jandova, I., Voll. R. E., and Glaser, C. 2021. “Case Report: Effective and Safe Treatment with Certolizumab Pegol, in Pregnant Patients with Cogans's Syndrome: A Report of Three Pregnancies in Two Patients,” Online, Frontiers in Immunology (Lausanne, Switzerland) 11:616992; Bhandari, G. S., Duggal, L., and Patel, J. N. 2019. “Cogan Syndrome,” An Autoimmune Eye and Ear Disease with Systemic Manifestations,” National Medical Journal of India 32(6): 349-351; Cabezas-Rodriguez, I., Brandy-Garcia, A., Rodriguez-Balsera, C., Rozas-Reyes, P., Fernandez-Llana, B., and Arboleya-Rodriguez, L. 2019. “Late-onset Cogan's Syndrome Associated with Large-vessel Vasculitis,” (in English and Spanish) Reumatologia Clinica (English Edition) 15(5): e30-e32; Scherg, F., Haag, F., and Krieger, T. 2019. “Off-label Application of Intravenous Immunoglobulin (IVIG) for Treatment of Cogan's Syndrome during Pregnancy,” Online, British Medical Journal Case Reports 12(10): e227915; Shamriz, O., Tal. Y., and Gross, M., 2018. “Autoimmune Inner Ear Disease: Immune Biomarkers, Audiovestibular Aspects, and Therapuetic Modalities of Cogan's Syndrome,” Online, Journal of Immunology Research 2918:1498640; Iliescu, D. A., Timaru, C. M., Batras, M., De Simone, A. and Stefan, C. 2015. “Cogan's Syndrome,” Online, Romanian Journal of Ophthalmology 59(1): 6-13; Sevgi, D. D., Sobrin, L., and Papaliodis, G. N. 2015. “Cogan Syndrome with Severe Medium and Large Vessel Vasculitis,” Online, Digital Journal of Ophthalmology 22(1): 32-34; Kessel, A. Vadasz, Z/, and Toubi, E. 2014. “Cogan Syndrome-Pathogenesis, Clinical Variants and Treatment Approaches,” Online, Autoimmunity Reviews 13(4-5): 351-354; Gasparovic, H., Djuric, Z., Bosnic, D., Petricevic, M., Brida, M., Dorlic, S. and Biocina, B. 2011. “Aortic Root Vasculitis Associated with Cogan's Syndrome,” Online, Annals of Thoracic Surgery 92(1): 340-341; The Merck Manual, 2006, “Corneal Disorders,” Whitehouse Station, New Jersey: Merck Research Laboratories Division, Merck and Company, Inc., chapter 102, page 899).

As one of the vasculitidies, in Cogan syndrome, injurious to the eye and inner ear, usually with negligible but sometimes unmistakable evidence elsewhere in the body, the symptoms are likely produced over time by the underlying vasculitis. To best articulate blood supplies of the sites most affected—the cornea and CN VIII, one might think it necessary to drug release jacket the artery feeding into the labyrinthine, or internal auditory artery, which is a branch of the anterior inferior cerebellar artery at the most proximate level where considerable neurosurgical intrusion would be necessary to gain access (Bordoni, B., Mankowski, N. L., and Daly, D. T. 2022. “Neuroanatomy, Cranial Nerve 8 (Vestibulocochlear), Online, Treasure Island, Florida: StatPearls Publishing).

However, unlike dopamine receptor-antagonist antipsychotics, for example, which pass through the blood brain barrier even in younger patients as those most susceptible to Cogan syndrome as a rheumatoid cerebral vasculitis, can differently affect the structures in the brain and even cause neuroleptic malignant syndrome (see, for example, Paul, T., Karam, A., Paul. T., Loh, H., and Ferrer, G. F. 2022. “A Case Report on Neuroleptic Malignant Syndrome (NMS): How to Approach an Early Diagnosis,” Online, Cureus (Palo Alto, California) 14(3): e23695; Simon, L. V., Hashmi, M. F., and Callahan, A. L. 2022. “Neuroleptic Malignant Syndrome,” Online, Treasure Island, Florida: StatPearls Publishing Co.; van Rensburg, R. and Decloedt, E. H. 2019. “An Approach to the Pharmacotherapy of Neuroleptic Malignant Syndrome,” Online, Psychopharmacology Bulletin 49(1): 84-91; Berman, B. D. 2011. “Neuroleptic Malignant Syndrome: A Review for Neurohospitalists,” Online, Neurohospitalist (Thousand Oaks, California) 1(1): 41-47), not all anti-inflammatoires cross the blood brain barrier to a significant degree.

Cogan's syndrome is an apt example of a condition where the target sites of symptoms—the cornea and statoacoustic nerve (vestibulocochlear nerve, audiovestibular nerve, auditory vestibular nerve, vestibular cochlear nerve, cranial nerve VIII, CN VIII)—would appear to defy local articulation, that is, not allow placement of a side-entry jacket through which drugs could be targeted to the sites affected. Targeting the cranial neurology is generally accomplished by jacketing and releasing medication into the internal carotids, which readily accessible, eliminates the risks associated with neurosurgical entry into the brain where to jacket an artery would likely cause more damage than it would overcome. On this basis, the internal carotids would be jacketed. Since the vasculitis is systemic, any entry of the drug into the general circulation should prove advantageous.

While the more pronounced adverse symptoms in Cogan syndrome which tend to capture the bulk of attention, are corneal and statoacoustic, the underlying vasculitis likely affects much of the brain. There are, therefore, two objectives to be considered. The first is to reduce the vasculitis, and the second to avoid provoking adverse side effects in nontargeted brain tissue while targeting the cornea and statoacoustic nerve.

Other sites disproportionately impaired by the vasculitis, such as the aortic root, can also be targeted. Where the systemic component of the vasculitis—with the potential to damage the aortic valve, for example, demands treatment, the object is to use that anti-inflammatory drug which is able to cross the blood brain barrier and treat both sites (see for example, Novakova, I., Subileau, E. A., Toegel, S., Gruber, D., Lachmann, B., and 4 others 2014. “Transport Rankings of Nonsteroidal Antiinflammatory Drugs Across Blood-brain-barrier in Vitro Models,” Online, PLoS [Public Library of Science] One 9(1): e86806).

Lipophilic statins such as simvastatin and atorvastatin pass through the blood brain barrier with simvastatin that most effective in this regard (see, for example, Wood, W. G., Eckert, G. P., Igbavboa, U., and Muller, W. E. 2010, Op cit., 10 paragraphs above). Moreover, the same statins have been determined to be potent anti-inflammatoires (see, for example, Lefer, D. J. 2002. “Statins as Potent Antiinflammatory Drugs,” Online, Circulation 106(16): 2041-2042) and effective in the treatment of vasculitis (see, for example, Tremoulet, A H. 2015. “The Role of Statins in Inflammatory Vasculitidies,” Online, 48(3): 177-180).

Furthermore, statins used topically have not been implicated in more than relatively innocuous, and reversible transient psychotropic effects, much less adverse cardiovascular (see, for example, Talreja, O., Kerndt, C. C., and Cassagnol, M. 2022. Online, “Simvastatin,” Treasure Island, Florida: StatPearls Publishing Co.) or gastrointestinal consequences (see, for example, Fujii, Nakabayashi, T., Hashimoto, S., and Kuwano, H. 2009. “Statin Use and Risk of Gastroduodenal Ulcer and Reflux Esophagitis,” Hepatogastroenterology 56(91-92): 641-644).

Simvastatin (sold under the trade names of Zocor and FloLipid) in an adequate dose delivered as targeted and cut off from the rest of the body for direct topical application should avoid the myopathy, myalgia, abdominal pain, gastritis, constipation, upper respiratory infections, impaired serum glucose control, or a significantly elevated aspartate aminotransferase indicative of damage to the liver, and possibly the pancreas, heart, or kidneys or elevated alanine aminotransferase indicative of damage to the liver (Talreja, O., Kerndt, C. C., and Cassagnol, M. 2022, Op. cit., preceding paragraph) of which statins are often accused, would therefore appear to be the drug least likely to cause adverse side effects when confined to the brain for targeting the cerebral microvasculature to suppress a vasculitis such as Cogan's.

### Targeting the Extracephalic Vasculature

Extracephalic symptoms of vasculitis in Cogan syndrome are usually nugatory, so that targeting the cerebral vasculature to suppress the ocular and statoacoustic symptoms should often constitute adequate treatment. However, when the extracephalic symptoms are serious, as when affecting the aortic valve or root, the disease calls for suppression throughout the rest of the body. For now, there is no corticosteroid that will suppress a vasculitis without the potential for causing adverse psychiatric, cardiovascular, or gastrointestinal, consequences. That understood, in the brain, needed is a drug that will intrinsically suppress the vasculitis with the least tendency to cause psychotropic disturbances or a stroke. The inducement of psychiatric consequences is a problem at higher doses in patients with preexisting psychiatric disorders, both of which circumstances are best avoided.

At the same time, the potential of the drug indicated, naproxen, to cause gastrointestinal injury can be countered by physically confining the drug to the target region so that it is denied any significant access to the digestive tract where it could cause erosions and ulceration (see, for example, Wongrakpanich, S., Wongrakpanich, A. Melhado, K., and Rangaswami, J. 2018. “Comprehensive Review of Non-steroidal Anti-inflammatory Drug Use in the Elderly,” Online, Aging and Disease 9(1): 143-150; Parepally, J. M. R, Mandula, H., and Smith, Q. R. 2006. “Brain Uptake of Nonsteroidal Anti-inflammatory Drugs: Ibuprofen, Flurbiprofen, and Indomethacin,” Online, Pharmaceutical Research 23(5): 873-881; Onder, G., Pellicciotti, F. Gambassi, G., and Bernabei, R. 2004. “NSAID-related Psychiatric Adverse Events: Who is at Risk?,” Drugs 64(23): 2619-2627; Nakamura, H. 2001. “Cyclooxygenase (COX)-2 Selective Inhibitors, Aspirin, A Dual COX-1/COX-2 Inhibitor, to COX-2 Selective Inhibitors,” Nihon Yakurigaku Zasshi (118(3): 219-230; Everts, B., Wahrborg, P. and Hedner, T. 2000. “COX-2 Specific Inhibitors—the Emergence of a New Class of Analgesic and Anti-inflammatory Drugs,” Clinical Rheumatology 18(5): 331-343).

Provided the dosing of nonsteroidal anti-inflammatory drugs which readily cross the blood brain barrier, such as flurbiprofen, lipopolysaccharide, and indomethacin to treat a cerebral vasculitis or cerebral angitis (see, for example, Parepally, J. M. R., Mandula, H., and Smith, Q. R 2006, Op cit., preceding paragraph) is kept to the safe maximum, the drug or drugs should calm the vascular lumina to differentially from a functional standpoint but uniformly and favorably improve the perfusion of the brain as a whole and therefore uniformly affect its different structures for the better (see, for example, Alajangi, H. K., Kaur, M., Sharma, A., Rana, S. Thakur, S. and 5 others 2022. “Blood-brain Barrier: Emerging Trends on Transport Models and New-age Strategies for Therapeutics Intervention Against Neurological Disorders,” Online, Molecular Brain 15(1): 48; Novakova, I., Subileau, E. A., Toegel, S., Gruber, D., Lachmann, B., and 4 others 2014, Op cit., above in this section; Berlit, P. 2010. “Diagnosis and Treatment of Cerebral Vasculitis,” Online, Therapeutic Advances in Neurological Disorders,” 3(1): 21-29-42; Parepally, J. M. R., Mandula, H., and Smith, Q. 2006. “Brain Uptake of Nonsteroidal Anti-inflammatory Drugs: Ibuprofen, Flubiprofen, an Indomethacin,” Online, Pharmaceutical Research 23(5): 873-881).

### Adverse Side Effects of Commonly Prescribed Immunosuppressive and Anti-Inflammatory Drugs

Immunosuppressives, which often also act as anti-inflammatoires, cannot be prescribed in sufficient amount or the susceptibility of the patient to infection would become too great, adrenal suppression possibly leading to death, hypertension, hyperglycemia, diabetes, cushingoid habitus, myopathy, aseptic bone necrosis, disturbed psychology, and other serious side effects (Lallana, E. C. and Fadul, C. E. 2011. “Toxicities of Immunosuppressive Treatment of Autoimmune Neurologic Diseases,” Online, Current Neuropharmacology 9(3): 468-477).

In contrast, the direct pipeline-targeting of drugs with topical efficacy or converted to post liver passage so as to function topically removes the restrictions on their dose or concentration, and if appropriate, a proper systemic dose can be concurrently released into a major vessel. Such a regimen is administered by the implanted disorder control system, which dispenses drugs both to specific sites and the entire body on the basis of implanted sensor indicia and continuously monitors the consequences to adjust the dosing or switch to a similar drug with a different side effect profile.

Sirolimus (rapamycin), which can cause gastritis; hyperlipidemia; arthralgia; cephalalgia; urinary tract infections; anemia, to include leukocytopenia, erythrocytopenia, and thrombocytopenia; hypertension; fever; nausea; diarrhea; constipation and impaction; peripheral edema; cognitive impairment; thrombogenesis, which can itself cause dyspnea, inflammation, pain, and confusion; and serum creatininosis, which can then in turn intensify the foregoing side effects as well as add fatigue, vomiting, muscle cramps, and uremic pericarditis (see, for example; Sallam, K., Bhumireddy, G. P., Evuri. V. D., Abella, J. P., Haddad. F., and 3 others 2021. “Sirolimus Adverse Event Profile in a Non-clinical Trial Cohort of Heart Transplantation Patients,” Annals of Transplantation 26: e923536-1-e923536-7; Asleh, R., Clavell. A. L., Pereira, N. L., Smith, B., Briasoulis, A., and 9 others 2019. “Incidence of Malignancies in Patients Treated with Sirolimus following Heart Transplantation,” Journal of the American College of Cardiology 73(21): 2676-2688; Ding, Y., Liang, X., Zhang, Y., Yi, L., Shum, H. C., and 8 others 2018. “Rap1 Deficiency-provoked Paracrine Dysfunction Impairs Immunosuppressive Potency of Mesenchymal Stem Cells in Allograft Rejection of Heart Transplantation,” Cell Death and Disease 9(3): 386; Wollard, J.-B., Kamar, N., Rousseau, A., Rostaing, L., Marquet, P. and Picard, N. 2012. “Association of Sirolimus Adverse Effects with mTOR [mammalian, or mechanistic, target of rapamycin] p70S6K or Raptor Polymorphisms in Kidney Transplant Recipients,” Pharmacogenetics and Genomics (Hagerstown, Maryland) 22(10): 725-732; Merkel, S., Mogilevskaja, N. Mengel, M., Haller, H., and Schwarz, A. 2006. “Side Effects of Sirolimus,” Transplantation Proceedings 38(3): 714-715); and its derivative

Everolimus, which less severe or persistent in its side effects than is sirolimus, can still cause epistaxis, dyspnea, weight loss, dysphagia, thoracalgia, hyperlipidemia, hyperglycemia, myelosuppression, rashes, pneumonitis, peripheral edema, nasopharyngitis, and stomatitis (see, for example; Arena, C., Bizzoca, M. E, Caponio, V. C. A., Troiano, G., Zhurakivska, K., Leuci, S., and Lo Muzio. L. 2021. “Everolimus Therapy and Side-effects: A Systemic Review and Meta-analysis,” International Journal of Oncology 59(1): 54; Horbelt, T. Kahl. A. L., Kole, F., Hetze, S., Wilde. B., Witzke, O., and Schedlowski, M. 2020. “Dose-dependent Acute Effects of Everolimus Administration on Immunological Neuroendocrine and Psychological Parameters in Healthy Men,” Clinical and Translational Science 13(6): 1251-1258; Arena, C., Troiano, G., Zhurakivska, K., Nocini, R., and Lo Muzio, L. 2019. “Stomatitis and Everolimus: A Review of Current Literature on 8.201 Patients,” Onco Targets and Therapy 12:9669-9683; Morviducci, L., Rota, F., Rizza, L., Di Giacinto, P., Ramponi, S., and 5 others 2018. “Everolimus is a New Anti-cancer Molecule: Metabolic Side Effects as Lipid Disorders and Hyperglycemia,” Diabetes Research and Clinical Practice 143:428-431; Potena, L., Pellegrini, C., Grigioni, F., Amarelli, C., Livi, U., and 8 others 2018. “Optimizing the Safety Profile of Everolimus by Delayed Initiation in de Novo Heart Transplant Recipients: Results of the Prospective Randomized Study EVERHEART [everolimus heart],” Transplantation 102(3): 493-501; Davies, M., Saxena, A., and Kingswood, J. C. 2017. “Management of Everolimus-associated Adverse Events in Patients with Tuberous Sclerosis Complex: A Practical Guide,” Orphanet Journal of Rare Diseases 12:35; Rashidi, M., Esmaily, S., Fiane, A. E., Gude, E. A., Tonseth, K., and 4 others 2016. “Wound Complications and Surgical Events in de Novo Heart Transplant Patients Treated with Everolimus: Post-hoc Analysis of the SCHEDULE [Scandinavian Heart Transplant Everolimus de Novo Trial with Early Calcineurin Avoidance] Trial,” International Journal of Cardiology 210:80-84; Paplomata, E., Zelnak, A., and O'Regan, R. 2013. “Everolimus: Side Effect Profile and Management of Toxicities in Breast Cancer,” Breast Cancer Research and Treatment 140(3): 453-462).

Tacrolimus, which can cause gastritis, nausea, vomiting, abnormal hepatic function, cephalalgia, insomnia, tremors, diabetes, electrolyte imbalances, increased body weight, diarrhea, arthralgia, blurred vision, tinnitus, otalgia, hyperglycemia, nephrotoxicity, pruritus, acne vulgaris, alopecia, angina pectoris, hypertension, arrhythmias, muscle cramps, bizarre or anxious dreams, jaundice, and acute renal failure (see, for example, Araya, A. A. and Tasnif, Y. 2021. “Tacrolimus,” Online, Treasure Island, Florida: StatPearls Publishing Co.; Kambara, H., Oyama, S., Inada, A., Niinomi, I. Wakabayashi. T., and Hosohata, K. 2021. “Current Status of Adverse Event Profile of Tacrolimus, in Patients with Solid Organ Transplantation from a Pharmacovigilance Study,” Journal of Clinical Pharmacology and Therapeutics 59(12): 753-759; Szpulska-Koziarska, D., Wilk. A., Marchelek-Mysliwiec, M, Sleboda-Teront, D., and Wisznewska, B. 2021. “Tacrolimus-based Immunosuppressive Therapy Influences Sex Hormone Profile in Renal-transplant Recipients—A Research Study,” Biology (Basel, Switzerland) 10(8): 709; Bentata, Y. 2020. “Tacrolimus: 20 Years of Use in Adult Kidney Transplantation. What We Should Know about its Nephrotoxicity,” Artificial Organs 44(2): 140-152; U.S. National Institute of Diabetes and Digestive and Kidney Diseases 2020. “Tacrolimus,” in LiverTox: Clinical and Research Information on Drug-induced Liver Injury (Online), Bethesda, Maryland: U.S. National Institute of Diabetes and Digestive and Kidney Diseases; Campagne, O., Mager, D. E., Brazeau, D. Venuto, R. C., and Tornatore, K. M. 2019. “The Impact of Tacrolimus Exposure on Extrarenal Adverse Effects in Adult Renal Transplant Recipients,” British Journal of Clinical Pharmacology 85(3): 516-529; Jouve. T., Noble, J., Rostaing, and Malvezzi. P. 2019. “An Update on the Safety of Tacrolimus in Kidney Transplant Recipients, with a Focus on Tacrolimus Minimization,” Expert Opinion on Drug Safety 18(4): 285-294; Teh, L. K., Dom, S. H., Zakaria, Z. A., and Salleh, M. Z. 2011. “A Systematic Review of the Adverse Effects of Tacrolimus in Organ Transplant Patients,” in Centre for Reviews and Dissemination (United Kingdom), Database of Abstracts and Reviews, York, England: University of York; Kemper, M. J., Sparta, G., Laube, G. F., Mozzari. M., and Neuhaus, T. J., 2003. “Neuropsychologic Side-effects of Tacrolimus in Pediatric Renal Transplantation,” Clinical Transplantation 17(20): 130-134).

Mycophenolate mofetil, which can cause gastrointestinal hemorrhage resulting in melena, diarrhea, hematuria, oliguria, hematochezia, cystitis, nausea, vomiting, cephalalgia, fatigue, vertigo, presyncope, disequilibrium, acute kidney injury, dyspepsia, paresthesias, thoracalgia, dyspnea, deviant blood pressure, leukocytopenia, erythrocytopenia, progressive multifocal leukoencephalopathy, tachycardia, peripheral edema, hyperglycemia, asthma, pleural effusion, diabetes, arthralgia, thrombosis, thrombophlebitis, gastritis, esophagitis, gastrointestinal hemorrhage; and in pregnancy, miscarriage, and teratological defects; and infrequently, malignant neoplasms, and pulmonary fibrosis (see, for example; American Society of Health-system Pharmacists 2022. “Mycophenolate Mofetil Side Effects,” Online, drugs.com/sfx/mycophenolate; American Society of Health-system Pharmacists 2021. “Mycophenolate,” Online, drugs.com/monograph, Tustumi, F., de Miranda, A. A., Neto, S. S. Jr., Fernandes, F. A., de Brito e Silva, M. B. and 7 others 2021. “Safety and Effectiveness of Mycophenolate Mofetil Associated with Tacrolimus for Liver Transplantation Immunosuppression: A Systematic Review and Meta-analysis of Randomized Controlled Trials,” Online, Clinics (Sao Paulo, Brazil) 76: e2597; U.S. National Institute of Diabetes and Digestive and Kidney Diseases 2020. “Mycophenolate,” in LiverTox: Clinical and Research Information on Drug-induced Liver Injury (Online), Bethesda, Maryland: U.S. National Institute of Diabetes and Digestive and Kidney Diseases; Dourakis, S. P., Boki, K., Soultati, A., Cherouvim, E. and Delladetsima, I. 2007. “Acute Hepatitis following Mycophenolate Mofetil Administration for ANCA [antinuclear-cytoplasmic antibody]-positive Vasculitis,” Scandinavian Journal of Rheumatology 36(3): 237-239; Loupy, A. Anglicheau, D., Mamzer-Bruneel, M. F., Martinez, F., Thervet, E., and 3 others 2006. “Mycophenolate Sodium-induced Hepatotoxicity: First Report” Transplantation 82(4): 581).

Cyclosporine (ciclosporin, cyclosporin) which can cause hypertension, hypertrichosis, paresthesias, tremors, nausea, cramps, gastralgia, bloating, diarrhea, cephalalgia, hematuria, oliguria, swelling in the face, gums, fingers, or lower legs, gingivitis, dyspnea, vomiting, lymphoproliferative malignancy, lymphoma, thoracalgia, melena, jaundice, lethargy, deviations in blood glucose level, acne, flushing, insomnia, gynecomastia, acid reflux, arthralgia, myalgia, depression, tachycardia, neurotoxicity, hepatotoxicity nephrotoxicity, and renal thrombus, among others (see, for example, Tapia, C., Nessel. T. A., and Zito, P. M. 2021. “Cyclosporine,” Online, Treasure Island, Florida: StatPearls Publishing Co.; U.S. National Institute of Diabetes and Digestive and Kidney Diseases 2020. “Cyclosporin,” in LiverTox: Clinical and Research Information on Drug-induced Liver Injury (Online), Bethesda, Maryland: U.S. National Institute of Diabetes and Digestive and Kidney Diseases; Teimouri. A., Ahmadi, S. R., Ardakani, S. A., and Foroughian, M. 2020. “Cyclosporine-A-based Immunosuppressive Therapy-induced Neurotoxicity: A Case Report,” Online, Open Access Emergency Medicine 12:93-97; Rezzani. R., 2004. “Cyclosporine A and Adverse Effects on Organs: Histochemical Studies,” Progress in Histochemistry and Cytochemistry (Amsterdam, North Holland, Netherlands) 39(2): 85-128); Kim, B. R., Yang S., Doh, E. J., Choi, C. W., and Youn, S. W. 2018. “Risk Factors Affecting Adverse Effects of Cyclosporine A in a Real-world Psoriasis Treatment,” Annals of Dermatology 30(2): 143-149; Crescenze, I. M., Tucky, B., Li, J., Moore, C., and Shoskes, D. 2017. “Efficacy, Side Effects, and Monitoring of Oral Cyclosporine in Interstitial Cystitis/Bladder Pain Syndrome,” Urology 107:49-54; Straathof, K. Anoop. P., Allwood, Z., Silva, J. Nikolajeva, O, and 4 others 2017. “Long-term Outcome following Cyclosporine-related Neurotoxicity in Paediatric Allogeneic Haematopoietic Stem Cell Transplantation,” Bone Marrow Transplantation 52(1): 159-162; Panicker, V. V., Mathew, A., and Dhamramaratnam, A. D. 2012. “Cosmetically Disfiguring Side Effects of Cyclosporine,” International Journal of Trichology 4(1): 50; Tedesco, D. and Haragsim, L. 2012. ‘Cyclosporine: A Review,” Journal of Transplantation 2012:230386; Asceric, M. Avdic, S., Nukic. S., and Vrabac-Mujcinagic, M 2007. “Intensive Observation of Toxic Side-effects after Several-year of Cyclosporin Treatment in Kidney Transplant Patient,” Bosnian Journal of Basic Medical Sciences (Mostar, Bosnia Herzegovina) 7(2): 132-135).

Methotrexate (amethopterin), which can cause hepatotoxicity, cirrhosis, renal failure, pancreatitis, interstitial pneumonitis, lymphoproliferative malignancy, ulcerative stomatitis, nausea, gastralgia (gastrodynia, stomachalgia), gastrointestinal hemorrhage resulting in hematemesis, and/or hematochezia or melena, hematuria, alopecia, anorexia, cephalalgia, gingivitis, conjunctivitis, nausea, vomiting, loss of appetite, dizziness, fatigue, vertigo, pyrexia (fever), chills, aphthous ulcers, leukocytopenia or leukopenia causing increased susceptibility to infection, leukoencephalopathy, myelopathy, neurotoxicity, mucositis, pneumotoxicity and pneumonitis, fibrosis and inflammation of the lungs, kidney failure, rarely pulmonary fibrosis, and if taken during pregnancy, it is a teratogen (see, for example, Ghannoum, M., Roberts, D. M., Goldfarb, D. S., Heldrup, J., Anseeuw, K., and 9 others 2022. “Extracorporeal Treatment for Methotrexate Poisoning,” Clinical Journal of the American Society of Nephrology 17(4): 602-622; Hannoodee, M. and Mittal, M. 2022. “Methotrexate,” Online, Treasure Island, Florida: StatPearls Publishing: Magdy, E. and Ali, S. 2021. “Stratification of Methotrexate-induced Oral Ulcers in Rheumatoid Arthritis Patients,” Special Care in Dentistry 41(3): 367-371; Schicchi, A., Scaravaggi, G., Petrolini, V. M., Malovini, A., Lonati, D., and 3 others 2020. “Poisoning Related to Therapeutic Error in Prolonged Low-dose Methotrexate Treatment,” British Journal of Clinical Pharmacology 87(5): 2385-2391: Gohar, A. 2019. “Response to ‘Reply to Gohar on “Lungs, Methotrexate and Psoriasis,” A Comment on “Fatal, Incidental, Idiopathic Pulmonary Fibrosis in a Patient Receiving Long-term Low-dose Methotrexate for Psoriasis,” Clinical and Experimental Dermatology 44(8): 948; Dalkilik, E., Coskun, B. N., Yagiz, B., Tufan, A. N., Ermurat, S. and Pehlivan, Y. 2018. “Methotrexate Intoxication: Beyond the Adverse Events,” International Journal of Rheumatic Diseases 21(8): 1557-1562; Shetty, A., Cho. W., Alazawi. W., and Syn, W. K. 2017. “Methotrexate Hepatotoxicity and the Impact of Nonalcoholic Fatty Liver Disease,” American Journal of Medical Sciences 354(2): 172-181; Huang, X. J., Jiang, Q. Chen, H., Xu. L., Liu, D., and 5 others 2005. “Low-dose Methotrexate for the Treatment of Graft-versus-host disease after allogeneic hematopoietic stem cell transplantation,” Bone Marrow Transplantation 36(3): 343-348; Costanzo, M. R., Koch, D. M., Fisher, S. G., Heroux, A. L., Kao, W. G., and Johnson, M. R. 1997. “Effects of Methotrexate on Acute Rejection and Cardiac Allograft Vasculopathy in Heart Transplant Recipients,” Journal of Heart and Lung Transplantation (New York, New York) 16(2): 169-178; Kremer, J. M., Petrillo, G. F., and Hamilton, R. A. 1995. “Pharmacokinetics and Renal Function in Patients with Rheumatoid Arthritis Receiving a Standard dose of Oral Weekly Methotrexate: Association with Significant Decreases in Creatinine Clearance and Renal Clearance of the Drug after 6 Months of Therapy,” Journal of Rheumatology 22(1): 38-40).

Prednisone/prednisolone, which can cause insomnia, cephalalgia, dizziness, nausea, vomiting, uneasiness, acne, increased appetite with weight gain, hyperglycemia, hypertension, edema; acne, osteoporosis, osteopenia, osteonecrosis leading to bone fractures; thyroid dysfunction, exophthalmos, or proptosis (bulging eyes), glaucoma, cataracts, adrenal suppression, cushingoid features such as moon facies, ‘buffalo hump, a buildup in belly fat; atrophy of the adrenals, or suprarenals, and hypoadrenalism (primary adrenal insufficiency, or Addison's disease with consequent hypotension, Cushing's syndrome, hyperglycemia, lethargy, impaired consciousness; dyslipidemia, hypertriglyceridemia, diabetes, bruising, weight gain, leukocytosis, delayed wound healing, skin atrophy, ecchymosis, myopathy of the extremities, epistaxis; hypoactive sexual desire, psychological depression or elevation, psychosis (hallucination, delusion, incoherent speech, inappropriate behavior), depression, osteoporosis, mood swings, changes in personality, insomnia, and in children, growth retardation. “Children with adrenal crisis secondary to adrenal suppression may present with hypotension, shock, decreased consciousness, lethargy, unexplained hypoglycemia, seizures, and even death,” (Yasir, M., Goyal, A., and Sonthalia, S. 2022. “Corticosteroid Adverse Effects,” Online, Treasure Island, Florida: StatPearls Publishing): (see also Hodgens A. and Sharman, T. 2022. “Corticosteroids,” Online, Treasure Island, Florida: StatPearls Publishing: Puckett, Y., Gabbar, A., and Bokhan, A. A. 2022. “Prednisone,” Online, Treasure Island, Florida: StatPearls Publishing: Fields, T. R. 2021. “Steroid Side Effects: How to Reduce Drug Side Effects of Corticosteroids,” Online at hss.edu/physicians_fields, New York, New York: Hospital for Special Surgery; Drozdowicz, L. B. and Bostwick, J. M. 2014. “Psychiatric Adverse Effects of Pediatric Corticosteroid Use,” Mayo Clinic Proceedings 89(6): 817-834; Bergmann, T. K., Barraclough, K. A., Lee, K. J., and Staatz, C. E. 2012. “Clinical Pharmacokinetics and Pharmacodynamics of Prednisolone and Prednisone in Solid Organ Transplantation,” Clinical Pharmacokinetics 51(11): 711-741; Stuart, F. A., Segal, T. Y., and Keady, S. 2005. “Adverse Psychological Effects of Corticosteroids in Children and Adolescents,” Archives of Disease in Childhood 90(5): 500-506).

Azathioprine/6-mercaptopurine, which can cause gastralgia, cephalalgia, thoracalgia, myalgia, hepatotoxicity, malignancy, especially skin cancer and lymphoma; arthralgia, chills, dizziness, diarrhea, rashes, nausea, vomiting, alopecia, alteration in color and texture of the hair; anorexia, or loss of appetite; weight loss, jaundice, skin rash, hematuria; melena or hematochezia; fatigue, weakness, bruising, aphthous ulcers; myelotoxicity and acute myelosuppression as pancytopenia, erythrocytopenia, or anemia; thrombocytopenia, leukocytopenia; acute pancreatitis, cough, fever; hoarseness; pharyngitis, or sore throat; swollen glands, peripheral edema, tracheobronchial edema, oliguria; painful urination, or dysuria; and rarely, constipation, tachycardia, dyspnea; and acholic (bile lacking), or grey stools. (see, for example, Mohammadi, O. and Kassim, T. A.2022. “Azathioprine,” Online, Treasure Island, Florida: StatPearls Publishing; Liew, D., Keith, C., Booth, J., and Perera, D. 2017. “Fatal Azathioprine Toxicity,” Online, Australian Prescriber 40(3): 109; U.S. National Institute of Diabetes and Digestive and Kidney Diseases 2017. “Azathioprine,” in LiverTax: Clinical and Research Information on Drug-induced Liver Injury (Online), Bethesda, Maryland: U.S. National Institute of Diabetes and Digestive and Kidney Diseases; Working Group on the Evaluation of Carcinogenic Risks to Humans 2012. “Azathioprine,” Online, Lyon, France: International Agency for Research on Cancer; Dogan, P., Grbovic. E., Inci, S., Bayraktar, F. and Cagli, K. 2015. “Azathioprine-induced Atrial Fibrillation,” Intractable and Rare Diseases Research 4(4): 207-309; James, A., Blagojevic, J., Benham, S. W., Cornall, R., and Frater, J. 2013. “Azathioprine Hypersensitivity Presenting as Septic Shock with Encephalopathy,” Online, British Medical Journal Case Reports 2013: ber2012008340; Turow, A. Yong. T. Y., Fok, J. S., and Li, J. Y. 2012. “Azathioprine Hypersensitivity Presenting as Cardiogenic Shock and Sweet's Syndrome in a Patient with Microscopic Polyangiitis,” Internal Medicine 51(14): 1889-1892; Bossuyt, P., Vergauwe, P., Inderadjaja, N. and Vermeire, S. 2007. “Azathioprine Induced Colitis: A Case Report and Review of the Literature,”Acta Gastro-Enterologica Belgica 70(3): 302-306; Demirtas-Ertan, G., Rowshani. A. T., and ten Berge, I. J. 2006. “Azathioprine-induced Shock in a Patient Suffering from Undifferentiated Erosive Oligoarthritis,” Netherlands Journal of Medicine 64 ((4): 124-126; Marinaki, A. M., Ansari, A., Duley, J. A. Arenas, M., Sumi, S., and 5 others 2004. “Adverse Drug Reactions to Azathioprine Therapy are Associated with Polymorphism in the Gene Encoding Inosine Triphosphate Pyrophosphatase (ITPase),” Pharmacogenetics and Genomics 14(3): 181-187; Fields, C. L., Robinson, J. W., Roy, T. M., Ossorio, M. A., and Byrd, R. P. Jr. 1998. “Hypersensitivity Reaction to Azathioprine,” Southern Medical Journal 91(5): 471-474; Brown, G., Boldt. C., Webb, J. G., and Halperin, L. 1997. “Azathioprine-induced Multisystem Organ Failure and Cardiogenic Shock,” Pharmacotherapy 17(4): 815-818; Knowles, S. R., Gupta, A. K., Shear, N. H., and Sauder, D. 1995. “Azathioprine Hypersensitivity-like Reactions-A Case Report and a Review of the Literature,” Clinical and Experimental Dermatology 20(4): 353-356; Ansley, A., Lennard, L., Mayou. S. C., and Kirby, J. D. 1992. “Pancytopenia Related to Azathiopr—ne-An Enzyme Deficiency Caused by a Common Genetic Polymorphism: A Review,” Journal of the Royal Society of Medicine 85(12): 752-756; Kissel, J. T., Levy, R. J., Mendell, J. R., and Griggs, R. C. 1986. “Azathioprine Toxicity in Neuromuscular Disease,” Online, Neurology 36(1): 35-39).

Ordinarily justifying systemic dispersal as well, statins (3-hydroxy-3-methyl-glutaryl-coenzyme A reductase) exhibit pleiotropic efficacy when directly applied topically to atheromatous lesions by direct targeting as well as by release into the general circulation. Means for the direct targeting of drugs is described and illustrated in U.S. Pat. Nos. 11,013,858, 11,389,171, and 11,759,186,002. In an elderly patient with cognitive decline and arteriosclerosis of the microvasculature, the automatic system also assures adherence to the prescribed drug regimen.

Used in moderation, statins and steroids have long been established as safe and effective in reducing the production of low-density lipoprotein and retarding cardiac allograft vasculopathy, acute rejection, and the development of malignancies. However, as is often necessary, both statins and steroids tend to become problematic when administered in larger doses over a long time—a circumstance quite eradicated by the discretionary targeting rather than systemic administration provided by a fully implanted prosthetic disorder response system.

Statins can become more problematic when the adverse consequences ordinarily associated with their use—mostly myalgia, myopathy, rarely, rhabdomyolysis, which can result in nephron obstruction with ischemia-induced trauma but not long-term renal dysfunction; and diabetes—are intensified when combined with other drugs, especially in patients with preexisting comorbidities (Szygula-Jurkiewicz, B., Szczurek, W., and Zembala, M. 2015, Op cit.), to include hypothyroidism and alcohol addiction. The benefits in treating arteriosclerosis of the microvasculature in relation to cognitive decline in old age is addressed below in this section.

Statins can cause not only myalgia and myositis but diabetes and hepatotoxicity, and when combined with other drugs such as immunosuppressives, calcium channel blockers, and azole antifungals, these side effects are often intensified. Of the statins, pravastatin has been established as that least interactive with other drugs (Ramkumar, S., Raghunath, A., and Raghunath, S. 2016. “Statin Therapy: Review of Safety and Potential Side Effects,” Online, Acta Cardiologica Sinica 32(6): 631-639).

Conditions such as interactions due to polypharmacy and preexisting comorbidities set aside, statins can cause cephalalgia, myalgia, insomnia, thrombocytopenia, indigestion, constipation, diarrhea, flatulence, dizziness, a sense of unwellness and fatigue, dizziness, type 2 diabetes with increased blood glucose, liver damage, nephrotoxicity, memory impairment, and confusion while the patient is kept on the statin (see, for example, Sizar, O., Khare, S., Jamil, R. T. and Talati, R. 2022. “Statin Medications,” Online, Treasure Island, Florida: StatPearls Publishing; Mach, F., Ray, K. K., Wiklund, O., Corsini, A., Catapano, A. L., and 22 others 2018. “Adverse Effects of Statin Therapy: Perception vs. the Evidence-Focus on Glucose Homeostasis, Cognitive, Renal, and Hepatic Function, Haemorrhagic Stroke and Cataract,” European Heart Journal 39(27): 2526-2539; Newman, C. B., Preiss, D., Tobert, J. A., Jacobson, T. A., Page, R. L. II, and 10 others 2018. “Statin Safety and Associated Adverse Events: A Scientific Statement from the American Heart Association,” Online, Arteriosclerosis, Thrombosis, and Vascular Biology 39: e38-e81; Pinal-Fernandez, I., Casal-Dominguez, M. and Mammen A. L. 2018. “Statins: Pros and Cons,” Medicina Clinica (Barcelona, Spain) 150(10): 398-402; Simic, I., and Reiner, Z. 2015. “Adverse Effects of Statins-Myths and Reality,” Current Pharmaceutical Design (Karachi, Pakistan) 21(9): 1220-1226; Beltowski, J., Wojcicka, G., and Jamroz-Wisniewska, A. 2009. “Adverse Effects of Statins-Mechanisms and Consequences,” Current Drug Safety 4(3): 209-228; Golomb, B. A. and Evans, M. A. 2008. “Statin Adverse Effects: A Review of the Literature and Evidence for a Mitochondrial Mechanism,” Online, American Journal of Cardiovascular Drugs 8(6): 373-418; Tuccori, M., Lapi, F., Testi, A., Coli, D., Moretti, U., and 7 others 2008. “Statin-associated Psychiatric Adverse Events: A Case/Non-case Evaluation of an Italian Database of Spontaneous Adverse Drug Reaction Reporting” Drug Safety 31(12): 1115-1123).

PCSK9 [proprotein convertase subtilisin/kexin type 9] inhibitors are for the most part less problematic than statins but not free of adverse side effects. If adverse neurocognitive effects are substantiated, their use in the elderly with arteriosclerotic microvasculature attributable dementia must be reevaluated. There is, however, no conclusive evidence to this effect (Bavry, A. A. 2021. “Further Cardiovascular Outcomes Research with PCKK9 Inhibition in Subjects with Elevated Risk-FOURIER,” Journal of the American College of Cardiology Online at acc.org/latest-in-cardiology; Mannarino, M. R., Sahebkar, A., Bianconi, V. Serban, M. C., Banach, M., and Pirro, M. 2018. “PCSK9 and Neurocognitive Function: Should It be Still an Issue after FOURIER [trial comparing the PCSK9 inhibitor evolocumab to placebo] AND EBBINGHAUS [trial to study evolocumab on cognitive function] Results,” Journal of Clinical Lipidology 12(5): 1123-1132; Giugliano, R. P., Mach, F., Zavitz, K., Krtz, C., Schneider, J., and 8 others 2017, “Design and Rationale of the EBBINGHAUS trial: A Phase 3, Double-blind, Placebo-controlled, Multicenter Study to Assess the Effect of Evolocumab on Cognitive Function in Patients with Clinically Evident Cardiovascular Disease and Receiving Statin Background Lipid-lowering Therapy—A Cognitive Study of Patients Enrolled in the FOURIER Trial,” Online, Clinical Cardiology 40(2): 59-65).

In familial hypercholesterolemia where a statin-intolerant patient needs a different means for reducing the level of circulating low-density lipoprotein, switching the patient to a PCSK9 [proprotein convertase subtilisin/kexin type 9] inhibitor while retaining ezetimibe can pose not just a problem of expense but also the risks of myalgia, arthralgia, both significantly increased when combined with a statin, cephalalgia, and muscle spasms.

PCSK9 inhibitors have also been cited for causing, nausea, diarrhea, injection site pain and sometimes, hematoma, muscle spasms, fatigue, influenza-like symptoms, nasopharyngitis, cognitive disorders, liver damage, skin rashes, and abdominal discomfort (see, for example, Ding, L., Chen, C., Yang, Y., Fang, J., Cao, L., and Liu, Y. 2022. “Adverse Events Associated with PCSK9 [proprotein convertase subtilisin/kexin type 9] Inhibitors: Disproportionality Analysis of the FDA Adverse Event Reporting System,” Online, Cardiovascular Therapeutics 2022:9866486; Ghernautan, V., Amini, M., and Sachmechi, 1., 2021. “Maculopapular Exanthema after the Second Dose of Evolocumab,” Online, Cureus 13(5): e15249; di Mauro, G., Zinzi, A., Scavone, C., Mascolo, A., Gaio, M., and 5 others 2021. “PCSK9 Inhibitors and Neurocognitive Adverse Drug Reactions: Analysis of Individual Case Safety Reports from the Eudra Vigilance Database,” Online, Drug Safety 44(3): 337-349; Sabatine, M. S., Giugliano, R P., and Schwartz, G. G., 2021. “Letter to the Editor re: “Serious Adverse Event and Deaths in PCSK9 Inhibitor Trials Reported on ClinicalTrials.gov: A Systematic Review,” Online, Expert Review in Clinical Pharmacology 14(2): 281-282; van Bruggen, F. H., Nijhuis, G. B. J., Zuidema, S. U., and Luijendijk, H. 2021. “Response to Letter to the Editor re: “Serious Adverse Events and Deaths in PCSK9 Inhibitor Trials Reported on ClinicalTrials.gov: A Systematic Review,” Online, Expert Review in Clinical Pharmacology 14(2): 283-284; Yuet, W. C., Ebert, D., and Jann, M. 2021. “Neurocognitive Effects Associated with Proprotein Convertase Subtilisin-Kexin Type 9 Inhibitor Use: A Narrative Review,” Online, Therapeutic Advances in Drug Safety 12:2042098620959271; van Bruggen, F. H., Nijhuis, G. B. J., Zuidema, S. U., and Luijendijk, H. 2020. “Serious Adverse Events and Deaths in PCSK9 Inhibitor Trials Reported on ClinicalTrials.gov: A Systematic Review,” Online, Expert Review in Clinical Pharmacology 13(7): 787-796; Zafar, Y., Sattar, Y., Ullah, W., Roomi, S., Rashid, M. U., Khan, M. S., and Schmidt, L. 2020. “Proprotein Convertase Subtilisin/Kexin Type-9 (PCSK-9) Inhibitors Induced Liver Injury—A Retrospective Analysis,” Online, Journal of Community Hospital Internal Medicine Perspectives 10(1): 32-37; Gurgoze, M. T., Muller-Hansma, A. H. G., Schreuder, M. M., Galema-Boers, A. M. H., Boersma, E., and Roeters van Lennep, J. E. 2019. “Adverse Events Associated with PCSK9 Inhibitors: A Real World Experience,” Online, Clinical Pharmacology and Therapeutics 105(2): 496-504; Swiger, K. J. and Martin, S. S., 2015. “PCSK9 Inhibitors and Neurocognitive Adverse Events: Exploring the FDA Directive and a Proposal for N-of-1 Trials,” Online, Drug Safety 38(6): 519-526).

Rituximab can cause and actually accelerate atheromatous degeneration and allograft vasculopathy following heart transplantation (Starling, R. C., Armstrong, B., Bridges, N. D., Eisen, H., Givertz, M. M., and 9 others 2019. “Accelerated Allograft Vasculopathy with Rituximab after Cardiac Transplantation,” Journal of the American College of Cardiology 74(1): 36-51). Passage across the blood brain barrier a basic issue, the subject is returned to in sections to follow.

### Automatic Drug Alternation and Rotation

Albeit intermittently to conserve rechargeable battery power, the process of automatically alternating between two drugs or gradually transitioning among a larger number of drugs as better to treat the condition over time is best practiced at a frequency consistent with the potential for damage by adverse side effects. Essentially, the system is devised and programmed to emulate the thought processes of a clinician, but with basic and considerable advantages. Unlike persisting in the use of a particular drug, then adding other drugs to suppress the adverse side effects of the primary drug, the system replaces the primary drug and will do so repeatedly whenever adverse side effects appear. Implanted in the patient, the prosthetic disorder response system detects and reacts to adverse effects immediately, not at the next scheduled visit to the clinic.

Moreover, unlike an accumulation of the adverse analyte to the degree that it generates an experiential correlate, that is, emerges into the conscious awareness of the patient, its detection occurs immediately. Otherwise, once felt, the patient must wait to see the doctor, often for a long time, while the dyscrasia progresses. In contrast, fully implanted in the patient and devised to function as if it were a part of his body, an integral component of his immune system, the system functions immediately and silently to correct the condition before the patient is even aware of it.

Often an implanted disorder response system will already have been placed due to a preexisting condition. If not already in place, and the condition to be treated is singular, a microcontroller chip-directed system is implanted endoscopically. By gradual transitioning is meant that the system gradually reduces the proportion in the drug delivery line of the offending drug and increases the proportion of the replacement drug. All drugs presenting adverse side effects, unless the patient is cured of the disease or disorder, this process is continued indefinitely.

Broadly, a simple microcontroller system is intended to more narrowly target treatment to a specific disorder such as portal or pulmonary hypertension with the aid of servocontrolled shunt to continuously tap off a volume of blood sufficient to reduce the blood pressure to within the normal range, for example. Integrated into a hierarchical control system, other diagnostic and therapeutic control arms are used to support this primary arm through the direct targeting of medication to the concomitant etiological and pathophysiological conditions responsible for and associated with the primary disorder.

Adverse side effects are usually proportional in severity to the size of the dose and duration of use. As a lipophilic statin, simvastatin for example, is beneficial in suppressing vasculitis (angitis, angiitis, arteritis) of the cerebral microvasculature and therefore retarding the development of dementia, but because cholesterol is essential to the formation and maintenance of myelin sheathing, supports mitochondrial and synaptic function, a reduction in its serum level may prove harmful. Direct pipeline targeting can remove restrictions on the level of the dose or its concentration, and switching to alternative drug can interdict the instatement of an adverse side effect.

All drugs have adverse side effects, usually in proportion to the dose and duration of use. As a lipophilic statin, simvastatin for example, is beneficial in retarding the development of dementia, but because cholesterol is essential to the formation and maintenance of myelin sheathing, supports mitochondrial and synaptic function, a reduction in its serum level may prove harmful. In some patients, a statin such as simvastatin is at one and the same time capable of protecting against dementia and inducing, albeit more irksome than serious in degree, cognitive impairment to include memory loss, uneasiness, and confusion usually reversed by stopping its use (Ellison, J. M. 2021. “Do Statins Increase or Decrease Alzheimer's Risk?,” Online, BrightFocus Foundation, brightfocus.org/alzheimers; Schultz, B. G., Patten, D. K., and Berlau, D. J. 2018. “The Role of Statins in Both Cognitive Impairment and Protection against Dementia: A Tale of Two Mechanisms,” Online, Translational Neurodegeneration 7:5; Suraweera, C., de Silva, V., and Hanwella, R., 2016. “Simvastatin-induced Cognitive Dysfunction: Two Case Reports,” Journal of Medicine Case Reports 10:83).

This capacity for both good and bad severely complicates the task of prescribing drugs. In those patients susceptible to both the good and the bad effects, it would be better if prescriptions could be rotated at intervals for the use of each keyed to gain the good while eluding the bad (see, for example, Canestero, W. J., Austin, M. A. and Thummel, K. E., 2014. “Genetic Factors Affecting Statin Concentrations and Subsequent Myopathy: A HUGENet [Human Genome Epidemiology Network] Systematic Review,” Online, Genetics in Medicine 16(11): 810-818). However, whether occupied and simply forgetful or cognitively impaired, pose a problem of adhering to a simple much less a prescription that calls for alternating among two or more drugs at exact intervals.

A capability for instant monitoring and controlling drug delivery at the same time represents a clear advancement in the practice of medicine and an elementary problem for a fully implanted prosthetic disorder response system. In simple disease, a rotating prescription can be assigned to a fully implanted chip microcontroller-directed drug delivery system consisting of the controller, small drug reservoirs subcutaneously positioned in the pectoral area, reservoir outlet pumps if necessary, tubes or catheters to deliver the drug or drugs to the target side, sensors appropriately positioned to send symptom data to the controller, and suitable connectors as described and illustrated in the interrelated set of patents specified in the opening section above. The system components are is implanted endoscopically and transluminally with the least dissection and trauma necessary.

As delineated in copending application Ser. No. 17/689,880, entitled Prosthetic Disorder Response Systems, in complex comorbid disease, each component disease is assigned to a different arm or axis of control in a hierarchical control system usually programmed using the Forth computer language. Briefly, the master control microprocessor in such a system is provided with a prescription-program and the implanted sensors needed to supply it with the responsive physiological data necessary to continuously evaluate and coordinate drug delivery in order to achieve the optimal homeostasis for the instant patient across the set of morbidities. In sum, by continuously recording the effect of administering one or a combination of drugs, the system can optimize drug delivery for that patient.

### Cognitive Side Effects Elusive of Detection

Psychological symptoms, headache, and indicia such as the cognitive effects of simvastatin not amenable to detection by chemical sensors or which would pose an inordinate expense to provide, are responded to by the patient who presses a switch on a wristband to suddenly switch or gradually transition to another drug. This does have the disadvantage that the side effect or effects are not eliminated before the patient becomes aware of the condition. The alternate drug also treats the disease, but whenever possible, is chosen as differing in chemistry, pharmacokinetics, and/or pharmacodynamics to least likely arouse the same side effect or effects. The system then switches from the one drug to the other until the condition subsides or resolves.

### Acquisition of Capability in the Surgical Treatment of the Heart

At the outset, pediatric cardiac surgery could no more than repair the severely malformed heart to the extent that at best, the patient would not die for a time. Pre-transplantation surgical treatment of the heart was limited to reconstruction using autologous tissue. The advent of heart transplantation then held out the prospect for a cure, but the realities of crude and traumatic surgical techniques and lifelong systemic immunosuppression soon disabused practitioners of this misperception. If severe and localized, pre-emergent pathophysiological conditions such a s metabolic should be interdicted through the prepositioning of an implanted response system that instantly detects and responds to symptoms by directly targeting counteractants to the site.

When defects are proximate to the solid organ to be transplanted, such as a double or an interrupted aortic arch associated with a severely deformed heart, then rather than to separately repair the aorta, the graft organ is best taken together with the affected vasculature. Intractable pulmonary vascular resistance may justify replacing the lungs as well as the heart. When the defects are disparate, such as pronounced cardiac defect and an abdominal aortic aneurysm, the automated concurrent repair of both can achieve a superior result in a single operation taking a fraction of the time otherwise needed to correct both in the same or different operations. By assigning a dedicated control axis (arm, channel) in a hierarchical control system to the diagnosis, repair and treatment of one defect among others, a master controller is used to coordinate the axes to optimize homeostasis across the set through the release of drugs or electrostimulation, for example.

Beyond a certain degree of function-eroding deviation, an insistence upon implementing measures to achieve survival however impaired for life the patient will remain postoperatively is ill-advised. Some genetic defects such as anencephaly and trisomies 13 and 18 are of such extensive and extreme severity that no chance for a normal future exists. Fewer than ten percent of neonates with severe malformities survive. Moreover, pleiotropic, unless a specific organ needed can be certified as mutation-free, such defects properly disqualify the organs of such patients for transplantation.

The ovum fertilized, the genome harboring such defects is immutably fixed as such without the possibility of correction, leaving an early death as the only satisfactory resolution. With more severe anomalies, the solution is prevention through in vitro fertilization and confirmation that the zygotes to be implanted in the endometrium are free of any serious mutations. Where the anomalies are less severe, and especially when the prospect of imparting normal function exists, the object is to accomplish this as expeditiously with as little discomfort and risk as possible. More specifically, the following demand the disallowance of halfway, and the immediate application of decisive corrective measures: 1. Genomic indication as to an impending metabolic defect or malignancy; 2. Emergence of a progressive disorder—in veterinary practice, a collapsed trachea; and 3. De facto malfunction through transplantation or the placement of a dependable prosthesis.

The shunning of invasive measures that once performed would likely eliminate significant disease, instead giving in to the more attractive but craven option of avoiding or forestalling more decisive measures through the palliative use of drugs, is for all but the oldest patients approaching death a form of dereliction. In the pediatric, it is an obsequious shirking of responsibility that will allow a degenerative process to proceed unabated through a life short, sick, and without fulfillment. Defects such as septal in an infant which can be repaired to satisfaction certainly do not warrant more decisive measures.

But a heart severely malformed, if even survivable, so that no possibility to initiate normal pulsatile circulation through surgical measures exists, should be addressed surgically or fitted with an assist device, cardiac neuromodulator, and suitable medication but only as a bridging strategy pending the availability of a good heart. To avoid the potential morbidity and even mortality posed by all, means used to treat heart failure and different cardiomyopathies, whether mechanical, electrical, medicinal, or a combination of these, demand expert application.

A compound vascular bypass, or sudden switched-organ heart transplant, and as will be described, a heterotopic double heart transplant in which both the recipient and donor hearts remain intact, requires lifelong immunosuppressive support frequently and generally cited as a significant deterrent to organ transplantation. However, backed up by a totally implanted automatic drug delivery system which assures pipeline targeted, correct dosing, much of this bias, even in the very young, the very old, and the mentally disturbed is dispelled. At the same time, the state of immunosuppressive pharmacy can be expected to advance. The potential toxicity of digoxin and the progressively degenerative changes induced as the result of the long-term use of a ventricular assist device, for example, pose considerably greater risk.

To overdrive a failing heart with a positive inotrope or through frequent electrical resynchronization shocks or positive inotropic stimulation of the left ventricle (see, for example, Reddy, V. Y., Petru, J., Malek, F., Stylos, L., Goedeke, S., and Neužil, P. 2020. “Novel Neuromodulation Approach to Improve Left Ventricular Contractility in Heart Failure: A First-in-Human Proof-of-Concept Study,” Circulation. Arrhythmia and Electrophysiology 13(11): e008407) certainly has greater potential for harm than does an orthotopic or heterotopic heart transplant carried out according to the method and means to be described.

To do so allows the minimization if not the elimination of medicinal and mechanical means all of which carry risks no less serious than any posed by transplantation. Electrical stimulation of the myocardium is not the sole means for affecting contractility through the application of neurological measures; cardiac sympathetic and renal nerve denervation and vagus and tragus nerve stimulation are also used (see, for example, Sobowale, C. O., Hori, Y., and Ajijola, O. A. 2020. “Neuromodulation Therapy in Heart Failure: Combined Use of Drugs and Devices,” Journal of Innovations in Cardiac Rhythm Management 11(7): 4151-4159).

A more biological, or natural, approach to support a failing heart is to provide it with a co-worker in the form of a second heart. FIG. 15A shows how the circulatory systems of the recipient and donor cooperate together. In the upper right-hand corner, the blood of the recipient passes straight up behind the valve diversion chute, while that of the donor passes over the chute to be redirected. This separation is why there is not an opposition in flow between the two streams despite the local increase in blood pressure.

Without electrical synchronization, the hearts will intermittently beat in synchrony, producing a transient increase in the local blood pressure where the streams converge. Midprocedural pressure relief at the donor-recipient bloodstream convergence points at the upper and lower corners in FIG. 15A are addressed below in section 2b(1)(b)(b16), entitled Direct Aortocaval Connection: Unified Entry Arterial and Exit Venous Confluence Conduits for the Heterotopic Connection of a Graft Heart in the Lower Abdomen.

Over the time it takes to transfer the donor heart into the circulatory system of the recipient, this should not pose a problem, certainly not with the aid of medication automatically dispensed by the implanted prosthetic disorder response system. In a double heart compound vascular bypass transplant, that synchronous beating by both hearts is infrequent and at least the innate heart is impaired as to its output pressure is favorable from the standpoint of avoiding a frequency of incidental pulses as would risk pulmonary hypertension. In no case should the coincidence of beats necessitate the placement of a passive pressure equalization take-off to a larger vessel.

Were a particular heart transplant exceptionally to prove unable to accommodate such intermittent pressure transients without the inducement of an adverse consequence, it is also possible to dispel the problem with a servovalve-regulated tuned shunt as addressed below in section 2b(2)(h)(h1), entitled The ‘Tuned’ Shunt. The servovalve is mounted in the direction opposite to that of the main or directional flow valve controlled by the same command signal. Draining into another large vessel wherein the diverted volume would little disturb, the shunt automatically draws off the necessary volume of blood to reduce the blood pressure to within the permissible range.

Use of a long-to-short confluence conduit with inline pump as described below in section 2b(1)(b)(b15), entitled Switchable Lines and Confluence Conduits: Long when Intercorporeal, to Short when Intracorporeal, Both Affording Connection of One or More bloodlines to the Same or a Different Number of Bloodlines with Self-leveling Apportionment of Outflow, and shown in FIG. 35B not only allows the distance to be spanned between donor and recipient to be much greater, the operative field to be made much clearer with fewer vessels to be individually tracked, and the confluent flow of the different input vessels to be medicinally treated alike, but can also serve to ameliorate any bothersome pressure transients.

In a compound vascular bypass heart transplant where the number of corresponding donor-recipient vessels to be connected is large a number of individual bloodlines and confluence conduits can be used simultaneously. This assumes, however, that exact synchrony of actuation of the inlet and outlet bistable stoppers 175 and 176 rotary solenoids respectively (not shown as behind but connected to axles 177) are actuated by a member of the surgical team from a single switch.

A manual key to simultaneously switch bistable stoppers 175 at the inlet and 176 at the outlet is limited to a single switchable line or confluence conduit per manual key and is reserved for use in case of a power loss at the time the lines and/or confluence conduits must be switched. Only a single bloodline or confluence conduit switchable by a manual key at a time, a number of keys must be used for members of the surgical crew to synchronize their action.

FIG. 15A shows the flow pattern whereby the graft organ is seamlessly switched from the circulatory system of the deceased donor and transferred into that of the recipient while continuously oxygenated and drained. For clarity, in this figure, all arterial vessels are schematically represented collectively at the top and all venous vessels collectively at the bottom. At the start of the procedure, blood flow is isolated in the donor and recipient circulatory systems which are completely separate.

Accordingly, the diversion chute, part number 18, of the valve at the upper right is fully extended to the left so that the valve is closed, all blood flow moving past the upper arterial shunt. Since the master control microprocessor moves all four valves simultaneously, at the start of the procedure, the diversion chute of the valve at the upper left is positioned all the way to the right so that no blood flows through the upper arterial shunt. This is because blood is blocked from entry into the shunt by valve obturator, part number 30 shown at the forward tip of each chute in valve detail FIGS. 2, 5, 7, 8, and 10A.

At the same time, the isolation of venous flow between donor and recipient circulatory systems is prevented by full retraction of the flow diversion chutes, hence, closure, of the venous valves at either end of the lower shunt. Specifically, the diversion chute of the valve at the lower right is fully retracted to the left and that of the valve at the lower left fully retracted to the right. In a solenoid-controlled valve transplant, the valves are suddenly shifted to the opposite ends of their throws to admit blood to flow through the shunts and fully join the donor and recipient circulatory systems.

In a servovalve-controlled metered compound bypass transplant, the valves are opened gradually with sensory inputs to signal the need for slowing or stopping and dispensing an anti-inflammatory. In an orthotopic heart transplant where the native or earlier transplanted heart is to be replaced in the chest, the valves will be fully opened at cross-clamping and harvesting of the donor organ. Within the time that life support can be entrusted to not significantly damage the blood of donor, this state of fully open reciprocal cross-circulation can be sustained to increase microchimerization.

In a heterotopic double heart transplant, depending upon the relative sizes and ejection fractions of the two hearts, the valves will be set to about half-way open when the donor heart is harvested and positioned in the recipient. This will almost always be in the chest to the right of the primary heart, the space necessary to accommodate the accessory heart provided as explained below in this section. Accordingly, as depicted in the front view of FIG. 15A with the recipient and donor positioned upright, side by side and head to head with the recipient on the right, the overall flow pattern of the blood blending circuit is clockwise.

FIG. 16 schematically shows the direct connections between corresponding vessels in the donor and recipient within the context of the overall flow pattern shown in FIG. 15A. Distributary vessels such as the coronaries, supraaortic brachiocephalic (innominate, brachiocephalic trunk), left common carotid, left subclavian, and the azygos veins are omitted as immediately supplied by the great vessels which unlike these are directly connected between the donor- and the recipient.

While the left pulmonary veins send oxygenated blood downward from the lungs into the heart, because the lungs are not shown in this drawing and because the veins are labeled below the heart, arrows to indicate the direction of flow have been omitted. In a metered transplant, the degree to which the blood of recipient and donor become blended is a function of the time this reciprocal cross-circulation flow pattern has been allowed to continue.

If left in progress long enough, the blood will eventually become half that of the recipient and half that of the donor. Exogenous blood and blood products can also be added during the process as can tolerance-inducing drugs. In a metered transplant, rejection sensors as addressed below in this section signal the master control microprocessor of a need to suspend continued blending and release immunosuppressive medication targeted to the pertinent valve or valves. As in a conventional heterotopic heart transplant, the hearts are left free-running, so that when they beat in alternation, the result appears as a false tachycardia wherein the successive pulses are of separate origin.

The apparatus and methods described herein almost entirely directed to exigent conditions, orthotopic and heterotopic double heart compound vascular bypass transplantation pertains to the replacement of a heart so congenitally malformed that its surgical reconstruction could accomplish no more than avert the death of the baby and in an adult, end-stage, not early heart failure. One or two ventricular assist pumps or left discussion here ventricular stimulators would create the impression of an elevated ejection fraction with excessive volume output. But because both hearts are somewhat impaired, the degree to which ejection is excessive should prove readily controllable at the highest.

Whereas a conventional heterotopic heart transplant seeks to provide the recipient with a competent left ventricle, a compound vascular bypass heterotopic transplant seeks to provide ventricles which by ‘joining forces,’ can achieve a normal ejection fraction. While observing the performance of either heart before the operation should give a reasonably good picture of how the two would work together, were the combined ejection fraction elevated to the extent that the pulmonary blood pressure could damage the alveoli, their epithelium, and capillary endothelium in a patient with normal lungs, this would be controlled by tapping off a portion of the pulmonary volume using valves of the types described herein and dividing it among other large vessels as is more natural and preferrable, or by programming the periodically pipe-line targeted administration of a negative inotrope.

Adding a second heart to share the burden is less demanding on the recipient heart than is causing the native a heart to work harder through the application of brute force (see, for example, Ahmad, T., Miller, P. E., Mccullough, M., Desai, N. R., and Riello, R. 2019. “Why Has Positive Inotropy Failed in Chronic Heart Failure? Lessons from Prior Inotrope Trials,” European Journal of Heart Failure 21(9): 1064-1078). In addition to sparing the cost of electronic devices—an important factor in third world countries—the adverse effects of these, delineated in the sections to follow, can be serious, and positive inotropes can pose some risk.

Accordingly, these can be highly advantageous in bridging to a replacement heart but are not ideal as end-repairs. Allowing the hearts to beat freely should prove satisfactory; if not the hearts can be assisted midprocedurally through the use of a reusable implantable cardioverter-defibrillator to control the net heartrate and electrostimulatory neuromodulators to adjust the absolute stroke volume, or ejection fraction, without or apart from the use of positive inotropes.

While the extended use of positive inotropes has been warned against (see, for example, Ahmad, T., Miller, P. E., Mccullough, M., Desai, N. R., and Riello, R. 2019, Op cit., second paragraph above in this section), exceptionally, in a compound vascular bypass heart transplant, the native heart of the recipient—about to be discarded in any event—may be treated with an exogenously administered positive inotrope (see, for example, Braunwald, E. 2005. “Normal and Abnormal Myocardial Function” in Kasper, D. L, Braunwald, E., Fauci, A. S., Hauser, S. L., Longo, D. L., and Jameson, J. L. Harrison's Principles of Internal Medicine, Chapter 215, page 1363, New York, New York: McGraw-Hill Medical Publishing Division) and/or electrical means.

Such measures to increase the ejection fraction over the short interval before the native heart is removed may empower the native heart to pump over the distance to the aorta in the donor heart. If not, as shown in FIGS. 32A, 32B, 35A, and 35B, a miniature inline pump is placed in the line. The benefits clearly outweighing the disadvantages, objection to, and therewith hindrance in the development of anatomicomedical, or simply medical surgery, meaning the positioning of sensors and drug releasing implant devices to directly target more serious localized disease, and in this way avoiding side effects in nontargeted tissue, even when plainly warranted, merely because it is invasive, is without justification.

Automated release is justified on the basis of directly piped drug targeting that avoids side effects and optimizes dosing—all the more beneficial with drugs that are costly—or for reasons of regimen adherence in the very young, the very old, or the uncooperative, or for any combination of reasons. The prepositioning of such a system can be executed with negligible trauma or risk of infection, certainly justified when an inaccessible root-site of disease that is the source of chronic ill health can be directly accessed without the need to medicate the entire body. What could be far better treated were it accessible is made immediately accessible at the expense of no more than a one-time, relatively brief procedure performed under local anesthesia.

A “two-edged sword,” the ability to squarely target immunosuppressives by direct pipeline delivery in concentrated dose to donor-recipient anastomoses, for example, optimizes the efficacy of these at the same time that it minimizes the susceptibility of the rest of the body to infection when these are allowed to circulate at the larger systemic dose required to compensate for the high degree of dilution. Infection secondary to the use of systemically dispersed immunosuppressives, rather than rejection of the graft organ, has often resulted in the death of an organ recipient. Immunosuppressives and chemotherapeutics, for example, can not only be squarely targeted and constrained to the intended target but an intentional lesser systemic dose can be controlled by any of several means delineated in the related applications specified above as well as in this application.

Mostly endoscopic, such a system, prescribed on the basis of genomic or other diagnostic data and described in U.S. Pat. No. 11,013,858, entitled Nonjacketing Side-entry Connectors and Prosthetic Disorder Response Systems, and U.S. Pat. No. 11,759,186, entitled Ductus Side-entry Jackets and Prosthetic Disorder Response Systems, can lie in wait to beset a progressive disorder the instant it emerges. Such a system can administer and provide treatment following an organ transplant, as well as subdue a chronic disease such as phenylketonuria or diabetes.

Pending the realization of an artificial pancreas—such a system can mitigate diabetes not by injection into tissue lying outside the bloodstream as does a continuous glucose monitor, but rather through the immediate detection of plasma hypoglycemia and the automatic proportional release directly into the circulatory system of glucose, or the immediate detection of hyperglycemia and the automatic proportional release of insulin and antihyperglycemic medication directly into the portal vein.

Conventional heart transplantation inflicts multiple insults of anoxia and trauma upon both the donor and recipient hearts that almost certainly play a role in pre-limiting the usable life of the graft organ to perhaps a dozen years. The donor heart must usually endure a cessation in perfusion associated with death followed by disconnection, hence, anoxia and the want of essential nutrients. Ex vivo perfusion can no more than ameliorate this trauma preceding the additional trauma of sectioning during transplantation. Then—contrary to the popular view that the old heart is simply cut out and the new one stitched in its place—both donor and recipient hearts are incised so that the anterior portion of the recipient heart is cut off to allow the anterior portion of the donor heart with competent ventricles to be stitched in its place.

A relatively minor disadvantage in this is that a more significant difference in the size of the donor and recipient hearts will make the donor heart unusable, which, had it been kept intact, would not be the case. The compound vascular bypass, or sudden switched-organ transplantation methods of solid organ transplantation completely eliminate this imposition of severe and compounded trauma. Instead, the intact replacement heart is completely spared the trauma associated with death, excision, storage, transection, and—albeit lessened where immune tolerance induction measures had been instituted—the shock of direct anastomosis of its cut edges to tissue subject to the immune system of someone else. Moreover, postoperatively the patient has the immediate support of the same totally implanted automatic disorder response control system that had administered the transplant and recorded the data that arose during that process.

Furthermore, there will be no such thing as noncompliance or nonadherence to the prescribed drug regimen. Implanted sensors detect a marshalling of lymphocytes, antibodies, and/or an elevation in temperature indicative of infection and the implanted control system instantly targets an antimicrobial to the site. A sign of rejection instantly prompts the release of immunosuppressive and anti-inflammatory counteractants. In this way, a subjective sense of the desirability to use a drug and the administration thereof are removed from dependence upon the competency and agreeability of the patient, whether an infant, demented, suicidal, contumacious, or psychotic.

The term ‘infusion’ having been restricted to denote delivery into a vein rather than into an artery as here intended, the more cumbersome ‘direct pipeline targeted arterial intravasation,’ or ‘direct pipeline targeting’ serve to denote the release by gravity as by infusion directly into the target blood supply through a side-entry jacket or an accessory channel thereof or an accessory channel incorporated into a vascular valve. When driven by a pump at the outlet of the drug reservoir rather than allowed to flow due to gravity, the term ‘injection’ is appropriate.

This limitation of the term ‘infuse’ to the intravenous as standard acknowledged, where the context makes it clear that release is into the arterial rather than the venous tree or that this distinction is irrelevant, the terms ‘infuse,’ ‘arterial infusion,’ or ‘arterial intravasation’ are used. Use of the term ‘direct pipeline targeting’ refers to delivery to a target structure, organ parenchyma, or volume of tissue by infusion through a prepositioned drugline from an implanted drug reservoir controlled by an implanted microcontroller or microprocessor.

The drugline may belong to a ductus side-entry jacket, nonjacketing side-entry connector, vascular valve or servovalve, or inline coupling jacket. Unlike any conventional means for administering medication, the automatic system can release a drug into the general circulation or pipeline-target the drug directly to any site immediately, at any time, if injected, then at any rate, and in any volume the implanted reservoir will support. With this level of maintenance following on a substantially trauma-free history, the result should prove far more durable than any following a conventional heart transplant.

Thus, the compound vascular bypass, or sudden switched-organ methods for solid organ transplantation not only eliminate the serious and damaging shortcomings of conventional transplantation before, during, and after the operation but materially improve each of these stages and unify them into a continuous process. To suppress abrupt snapping, impact, and clicking noise when actuated, plunger solenoid-driven valves are highly damped. All components used in performing a compound vascular bypass, or sudden switched-organ solid organ transplantation must be nonsparking as could ignite oxygen.

The central object in the design of all vascular valves, to include side-entry, inline coupling, and inline coupling jacket bridge-mounted diversion jackets, valves, and servovalves is to allow placement in the vascular tree without the loss of or the need to suspend the flow of blood through the vessel treated thus. This one capability alone eliminates numerous sources of midoperative complications and postoperative sequelae associated with both the stoppage of blood flow per se and the means employed to accomplish the stoppage.

In the chest, inline coupling jacket-secured tie-lines, or bridges, for replacing diseased segments along the aortic arch, to include its supra-aortic branches—innominate, or brachiocephalic, left common carotid, and left subclavian arteries—for example, can be provided in standardized embodiments to replace even extensive sections of the thoracic, thoracoabdominal, or abdominal aorta quickly and automatically without interruption in blood flow or bleeding. Anatomical shaping in this regard consists of including branches where all parts of the prosthesis are cylindrical without the mild tapering or flaring seen in the actual anatomy.

The same is no less true of the abdominal aorta, where depending upon the origin and destination levels of the segment to be replaced, branches for the visceral—to include the celiac, superior and inferior mesenteric, renal and gonadal arteries—and parietal branches, to include the inferior phrenics, lumbars, and middle sacral arteries are included in the prosthesis. The abdominal aorta should prove replaceable to a considerable distance inferior to the origin of the common iliacs. Broadly, application to vessels of lesser caliber may be limited by the need for excessive dissection to encircle the vessel; however, smaller vessels are rarely affected by defects as would require their replacement, which is bome out by the lack of comparable forms of correction, such as the transluminal insertion of an endostent, or stent-graft to treat these.

Inline coupling jackets, addressed below in section 2b(2)(b), entitled Vascular Segment Prostheses: Inline Coupling Jackets and Bridging, or ties, and shown in FIGS. 36A, 36E, 37A, and 37D are one-time/one-step insertion flow-switch valves which transect and reconnect the stumps of a native to a prosthetic replacement ductus. Inline coupling jackets are used in pairs at either end of the prosthetic segment they carry behind a cutting tool to both remove and replace a segment along a ductus, typically, a great vessel. Since it is a segment removed, the jacket at either end and the conduit connecting these constitute a unit device.

Characterization of inline coupling jackets as valves rather than switches could justified on the basis that prosthesis insertion can be stopped at any moment, leaving flow divided in any proportion between the innate and prosthetic paths; however, the practical use thereof does not comprehend such an apportioning of blood flow between the native and prosthetic lumina for the brief interval during which this occurs. The native and its replacement segment, or prosthesis, may be simple, without branches, or include branches or an intervening component such as a confluence conduit or a choke or servovalve.

In either case, the prosthesis replacing the native segment is a tie-line. When the segment removed and replaced includes branches, smaller individual inline coupling jackets are used to transect and replace the distal ends of the segments of the branches proximal to the primary vessel of these as well. In this case, the entry jacket with the branch segment connecting jacket represent the entry portion along the tie-line. What might be referred to as the iconic example that best illustrates the value of an anatomically shaped tie-line to include branches is one used to replace the aortic arch of a neonate born with a severe defect due to one of the syndromes with aortic involvement. Use of the term ‘anatomical’ means that any branches are included in the prosthesis so that it is made separately from the reel or coil of unbranched stock whether expandable or not.

Due to the rate and extent of growth in a neonate, such a repair can never achieve durability, the prospect of complications, whether due to dehiscence, migration, stenosis, redilatation, and the risk of rupture and sudden death perpetually abiding. Directly receiving the powerful pulsatile outflow from the left ventricle, the forces to which the walls of the aortic arch are continuously exposed are the largest in the circulatory system.

For this reason, a successful prosthetic aortic arch—one that will grow with the patient without dehiscing, rupturing, or dissecting—is certainly adaptable to provide a comparable prosthesis for replacing the abdominal aorta where the anatomy and forces are far less challenging than the crowded and constantly changing geometry of the great vessels where congenital rather than acquired defects that necessitate the ability to accommodate growth prevail. Several congenital syndromes are associated with a defective structure in the walls of major arteries—in particular, the aorta, and the numerous causes for pulmonary hypertension set aside, the main pulmonary artery, or pulmonary trunk, which receives blood under pressure directly from the right ventricle.

Syndromes with aortic involvement invariably include nonaortic defects and comorbidities which make these ideal subjects for the application of a fully implanted automatic disorder response system as addressed in copending application Ser. No. 17/689,880, entitled Prosthetic Disorder Response Systems. While including defects outside the scope of internal medicine that call for surgical repair, such as craniofacial and maxillofacial defects-often a cleft palate, and developmental delay, other associated comorbid conditions such as autoimmune diseases, and Graves” (Flajani-Basedow-Graves, toxic diffuse goiter) disease are ideally suited for the continuous monitoring and response of a fully implanted automatic ambulatory disorder response system.

Less obscure syndromes responsible for or associated with defects of the aorta and other great vessels include disorders characterized by the defective development of connective tissue such as Marfans syndrome, Ehlers-Danlos vascular, or nonhypermobile Type IV syndrome, and Loeys-Dietz syndrome (aortic aneurysm syndrome due to anomalies in transforming growth factor beta receptors; and other than skin disorders per se, such as Tumer (gonadal dysgenesis, Ullrich-Tumer, Bonnevie-Ullrich, Bonnevie-Ullrich-Tumer, 45,X, 45,X0 syndrome), autosomal dominant polycystic kidney disease (adult-onset kidney disease), type 1 neurofibromatosis (von Recklinghausen disease, morbus Recklinghausen); Williams (Williams-Beuren, Williams-Beuren-Beuren) syndrome; Alagille syndrome (Alagille-Watson syndrome, hepatic ductular hypoplasia); and DiGeorge syndrome (DiGeorge anomaly, Shprintzen-Goldberg syndrome, Takao syndrome, Sedlackova syndrome, Cayler cardiofacial syndrome, conotruncal anomaly face syndrome, velocardiofacial, CATCH 22, chromosome 22q11.2 deletion syndrome)(Zucker, E. J. 2018. “Syndromes with Aortic Involvement: Pictorial Review,” Cardiovascular Diagnosis and Therapy 8 (Supplement 1): S71-S81). A congenital vasculitis which can lead to multiple successive defects of the great vessels recommending ‘extension for prevention’ by placement of the wider vessel with a synthetic prosthesis provided herein as a tie-line type anatomical replacement addressed below in section 2b(2)(c), entitled Vascular Segment Prostheses: Inline Coupling Jackets and Bridging, or Tie-lines and its subordinate sections is Behçet's disease. Both of these types of congenital causes are referred to below in this section.

Thoracic aortic involvement amenable to repair with a prosthesis of the kind addressed below in section 2b(2)(f), entitled Expandable Structure to Accommodate Growth and Self-adjust in Size in Vascular Segment Prostheses, Lines, and Confluence Conduits Placed in Children, is sufficiently extracardiac, or downstream from the root, to allow the placement of an inlet entry inline coupling jacket as addressed below in sections 2b(2)(c)(c1), entitled Inline Coupling Jackets Proper, and 2b (2)(c), entitled Vascular Segment Prostheses: Inline Coupling Jackets and Bridging, or Tie-lines.

Especially in the young, hearts with internal defects incapable of adequate repair as would institute normal circulation and development would be advantageously replaced using the direct donor to recipient circulatory system transfer compound vascular bypass technique addressed below in section 2b(1)(b), entitled Metered Compound Vascular Bypass, or Metered Switched-organ, Transplantation. Due to the elimination of ischemia, the very considerable reduction in trauma, and postoperative support by a fully implanted prosthetic disorder response system, the pool of available organs as well as the pool of prospective recipients who can meet a less stringent standard of eligibility is considerably enlarged.

The requirement for expansion with growth eliminated as a prerequisite, acquired defects in adults such as aneurysms and dissections of major vessels are less challenging to fix with a prosthesis. Section 2b(2)(f) below entitled Expandable Structure to Accommodate Growth and Self-adjust in Size in Vascular Segment Prostheses, Lines, and Confluence Conduits Placed in Children, describes, and FIGS. 36A and 37F show an expandable prosthesis which the operator can manually expand prior to placement to fit all but very large adults.

The walls of a major vessel such as the aorta structurally, that is, materially, defective despite repair that had been limited to the defect—something a competent operator would never allow—as such without replacing the aorta in its entirety is likely to be followed by another aneurysm in the thoracic, thoracoabdominal, or abdominal aorta, and attempts at repair, none of which can accommodate growth, are susceptible to stenosis, migration, dehiscence, redilatation, and dissections. In such a circumstance, the proper object is to replace the aorta with an expandable replacement made of a material having a tensile strength, hence, a resistance to rupture, or bursting, greater by far than the body could ever generate.

The need for endothelial function generally coextensive with the endothelium, the excision of endothelium should have little impact on other endothelium. However, if the specific case indicates a need for the automatic release of vasodilators in response to a heightened activity level of the patient, compensation for the loss or reduction in endothelium and its function is readily detected and responded to by an implanted prosthetic disorder response system. The drugline, or accessory channel, provided with every inline coupling jacket vascular prosthesis allows the direct pipe-targeted delivery of medication or an anticoagulant, for example, and can be tied to carotid and aortic artery body output to be synchronized with the activity level of the patient.

### Arterial Defects

As applies to much of the lower body where the vasculature such as the femoral arteries are enclosed within skeletal muscle with space lacking, or where to use a tie-line as described below in section 2b(2)(c), entitled Vascular Segment Prostheses: Inline coupling jackets and Bridging, or Tie-lines, would necessitate too much dissection, and/or the potential for leakage is less, the placement of a conventional endoluminal stent or stent-graft, which placed transluminally also eliminates the need for a large surgical entry wound, and is to be preferred (see, for example, Jalili, J., Ebrahiminik, H., Zafarani, F., and Akhavi Milani A. 2022. “Endovascular Treatment of the Ruptured Pseudoaneurysm of Common Femoral Artery in Behçet's Disease,” Vascular and Endovascular Surgery 56(6): 616-621).

A need for repair of a key artery or vein is always an emergency. In a neonate, the cause will likely be a congenital defect, while in an older patient the cause will more often be an accident in combination or not with a congenital defect. While the placement of an endoluminal stent-graft in an adult spares the patient the need for a large and painful entry wound and its potential complications, conventional repair thus can develop an endoleak necessitating at least endoscopic if not open reentry.

Especially in a neonate or small child—especially one with a connective tissue disorder—a large artery such as one aneurysmal, stenotic, or affected by a pseudoaneurysm, dissection, or some other congenital or acquired defect would fare better were the affected segment replaced with a sutureless prosthesis substantially incapable of developing a secondary defect such as an endoleak, which albeit necessitating an open procedure, would expand in step with growth and thus avoid the need for painful revision. Prostheses of this kind are described below in section 2b(2)(f), entitled Expandable Structure to Accommodate Growth and Self-adjust in Size in Vascular Segment Prostheses, Lines, and Confluence Conduits Placed in Children.

Where a recurrence of the defect in up- or downstream tissue is likely, the eventuality is precluded by placing a prosthesis that includes that area or another prosthesis to cover that area (see, for example, Akkuzu, G., Özgür, D. S., Karaalioğlu, B., Yalçin Mutlu, M., Yildinim, F., and 3 others 2023. “Behçet's Syndrome Resembling Takayasu's Arteritis with the Distribution of Arterial Involvement: A Case Report and Literature Review,” Online, European Journal of Rheumatology (Istanbul, Turkey) 10(2): 62-66; Chen, G. X., Wan, J. H., Chen, C. W., and Huang, B. 2023. “Hybrid Surgery Management Challenges of a Behçet's Disease Patient with Recurrence of Aortic Aneurysms: A Case Report,” Online, Frontiers in Cardiovascular Medicine (Lausanne, Switzerland) 10:1097129; Faiza, Z. and Sharman, T. 2022. “Thoracic Aorta Aneurysm,” Treasure Island, Florida: StatPearls Publishing Co.; Mehta, A., Shakarchi, J. A., Meecham, L., Vijay, S., and Downing, R. 2021. “Undiagnosed Behçet's Disease Complicated by Multiple Pseudoaneurysms and COVID-19 Infection,” Online, International Journal of Angiology (New York, New York) 32(1): 71- 74; Yamamoto, Y., Inoue, Y., Ichinose, T., Nishizawa, M., Igari, K., Toyofuku, T., and Kudo, T. 2018. “Multiple Recurrent Pseudoaneurysms after Endovascular Repair of Abdominal Aortic Aneurysm in a Patient with Behçet's Disease,” Online, Annals of Thoracic and Cardiovascular Surgery 24(6): 315-319, Oliveira, L., Torres, E., and Anjos, R. 2015. “Abdominal Aorta Interruption and CHD (congenital heart disease),” Cardiology in the Young (Cambridge, England) 25(5): 992-993; Lacombe, M. 2005. “Congenital Malformations of the Abdominal Aorta,” (in French with English abstract at Pubmed), Bulletin de l'Academie Nationale de Medecine 189(2): 359-371; Chiesa, R., Melissano, G., Mariani, M. A., Castrucci, M., and Grossi, A. 1991. “Coarctation and Hypoplasia of the Abdominal Aorta,” (in Italian with English abstract at Pubmed), Minerva Chirurgica 46(21-22): 1197-1204).

The repair of congenital or acquired defects along a critical artery where a rupture if not immediately closed would likely result in death pertains to the aortic arch with its supraaortic branches commonly affected thus (see, for example, Cury, M., Greenberg, R. K., Morales, J. P. Mohabbat, W., and Hernandez, A. V. 2009. “Supra-aortic Vessels Aneurysms: Diagnosis and Prompt Intervention,” Online, Journal of Vascular Surgery (New York, New York) 49(1): 4-10). An alternative treatment where the defect is relatively small is a bypass such as shown in FIG. 22C or a tuned shunt such as addressed below in section 2b(2)(h), entitled Local Vascular Pressure Relief, and shown in FIG. 22F to a branch of the affected artery with excision of the defect (see, for example, Johari, H. G., Moein, S. A., Shenavande, S., Amirian, A., and Nabavizadeh, S. S. 2022. “Ruptured Innominate Artery Pseudoaneurysm Presenting as Hoarseness in Behçet's Syndrome: A Case Report,” Online, Journal of Medical Case Reports 16(1): 439).

Conventional repair of the thoracic aorta by sutured patch aortoplasty, elephant trunk, or frozen elephant trunk or of the abdominal aorta with an endoluminal stent graft are susceptible to failure and incapable of expansion sufficient to accommodate growth in a neonate or small child who would better fare were the affected segment if not the entire thoracic aorta to include its supraaortic distributaries replaced. As a minimally invasive alternative most likely to preserve the aortic chemoreceptive bodies and paraaortic bodies which comprise chromaffin cells, thoracic endovascular aneurysm repair thoracic endovascular aortic repair (TEVAR) provides a significant advantage but cannot replace the supra-aortic branches to eliminate postoperative complications and is associated with the postoperative formation of dissections that necessitate further repair.

Replacement of the aortic arch to include the supra-aortic branches with a seamless unitary prosthesis incapable of migration or leaking as addressed below in section 2b(2)(c), entitled Vascular Segment Prostheses: Inline Coupling Jackets and Bridging, or Tie-lines and shown in FIG. 36F requires an open surgical field; however, it is likely to eliminate postoperative complications.

In both pediatric and adult patients, replacement whether automatic as described below in section 2b(2)(c)(c2), entitled Automatic Insertion into a Ductus of an Inline Coupling Jacket-secured Bridging, or Tie-line and Its Replacement if Necessary, with a seamless prosthesis unsusceptible to the development of leaks and in a child, capable of expansion, eliminates the need for revision for many years if not life (additional references below in section 2b(2)(c), entitled Vascular Segment Prostheses: Inline Coupling Jackets and Bridging, or Tie-lines; see, for example, Achaya, M., Sherzad, H., Bashir, M., and Mariscalo, G. 2022. “The Frozen Elephant Trunk Procedure: Indications, Outcomes, and Future Directions,” Online, Cardiovascular Diagnosis and Therapy 12(5): 708-721; Andic. M., Mustafi, M., Bonorden, C., Grozinger, G., Artzner, C., Schlensak, C., and Lescan, M. 2022. “Longitudinal Morphological Changes of the Aorta and the Endograft Position before and after Distal Stent Graft-induced New Entry in Aortic Dissections,” European Journal of Cardiothoracic Surgery 63(1): ezac547; Berger, T., Graap. M., Rulski, B., Fagu, A., Gottardi, R., and 6 others 2022. “Distal Aortic Failure following the Frozen Elephant Trunk Procedure for Aortic Dissection, Online, Frontiers in Cardiovascular Medicine 9:911548; Howard, C., Al-Tawil, M., Geragotellis, A., and Abdelhaliem, A. 2022. “Adverse Complications of Frozen Elephant Trunk, Do We Have Enough Quality Data?,” Journal of Cardiac Surgery 37(11): 3863-3864; Jubouri, M., Kayali, F., Saha, P., Ansari, D. M., Rezaei, Y., and 5 others 2022. “Incidence of Distal Stent Graft Induced New Entry vs. Aortic Remodeling Associated with Frozen Elephant Trunk,” Online, Frontiers in Cardiovascular Medicine 9:875078; Kayali, F., Qutaishat, S., Jubouri, M., Chikhal, R., Tan, S. Z. C. P., and Bashir, M. 2022. “Kinking of Frozen Elephant Trunk Hybrid Prostheses: Incidence, Mechanism, and Management,” Online, Frontiers in Cardiovascular Medicine 9:912071; Kreibich, M., Berger, T., Walter, T., Potratz, P., Discher, P., and 6 others 2022. “Downstream Thoracic Endovascular Aortic Repair following the Frozen Elephant Trunk Procedure,” Online, Cardiovascular Diagnosis and Therapy 12(3): 272-277; Nakhaei, P., Bashir, M., Jubouri, M., Banar, S. Ilkhani. S. and 6 others 2022. “Aortic Remodeling, Distal Stent-graft Induced New Entry and Endoleak following Frozen Elephant Trunk: A Systematic Review and Meta-analysis,” Journal of Cardiac Surgery 37(11): 3848-3862; Nomura, Y., Tonoki, S., Kawashima, M., Fujisue, J., Uchino, G., and 5 others 2021. “Distal Stent Graft-induced New Entry after Total Arch Replacement with Frozen Elephant Trunk for Aortic Dissection,” Online, Annals of Vascular Diseases (Tokyo, Japan) 14(4): 362-367; Czerny, M., Eggebrecht, H., Rousseau, H., Mouroz, P. R., Janosi, R. A., and 23 others 2020. “Distal Stent Graft-induced New Entry after TEVAR [Thoracic Endovascular Aortic Repair] or FET [Frozen Elephant Trunk]: Insights into a New Disease from EuREC [European Registry of Endovascular Aortic Repair Complications],” 110(5): 1494-1500; Song. S. B., Wu, X. J., Sun, Y., Cai. S. H., Hu, P. Y., and Qiang, H. F. 2020. “A Modified Frozen Elephant Trunk Technique for Acute Stanford Type A Aortic Dissection,” Online, Journal of Cardiothoracic Surgery 15(1): 322; Fiorucci, B. and Tsilmaparis, N. 2017. “Fenestrated Endovascular Aneurysm Repair of a Stent Graft-induced New Entry Tear in a Patient with Celiacomesenteric Trunk,” European Journal of Vascular and Endovascular Surgery 53(2): 184; Janosi. A., Tsagakis, K., Bettin, M., Kahlert, P., Horacek, M., and 5 others 2015. “Thoracic Aortic Aneurysm Expansion Due to Late Distal Stent Graft-induced New Entry,” Catheterization and Cardiovascular Interventions 85(2): E43-E53; Kim, J. H. and Na, C. Y. 2012. “Elephant Trunk Fabric Bleeding during Second-stage Thoraco-abdominal Aneurysm Repair,” Online, Interactive Cardiovascular and Thoracic Surgery 15(4): 792-793; Jonker, F. J., Schlosser, F. J., Geirsson, A., Sumpio, B. E., Moll, F. L. and Muhs, B. E. 2010. “Endograft Collapse after Thoracic Endovascular Aortic Repair” Journal of Endovascular Therapy 17(6): 725-734; Neri. E., Toscano, T., Frati, G., and Sassi, C. 2001. “The Elephant Trunk Technique: A New Complication,” Online, Texas Heart Institute Journal 28(3): 220-222; additional references cited in this section).

Following relatively localized (focal, in situ) treatment such as an angioplasty or angioplasty and stenting-which in distending the lumen can further weaken the arterial wall-even less extensive defects may restenose, aneurysmize, reaneurysmize, pseudoaneurysmize, or incise. If not in utero, these sequelae indicate that the cause had likely been a histological malconstitution of the arterial walls during development or disease-induced degradation which resulted in a lack of adequate strength.

Congenital connective tissue disorders such as vascular type Ehlers-Danlos syndrome or autosomal recessive cutis laxa and others specified above in this section can result in congenital defects of malformity of the thoracic aorta such as an aneurysm as with Marfan and para-Marfan syndromes, which can cause an irreversible dilatation with the risks of rupture and dissection.

No less than its diameter, the pattern of blood flow through the thoracic aorta, to include shear stress, helicity, and flow displacement, should be diagnosed as signaling a need for preemptive repair (Yousefi, P. Sharma, R., Figueroa, C. A., and Jahangiri. M. 2017. “Functional Assessment of Thoracic Aortic Aneurysms—The Future of Risk Prediction?,” Online, British Medical Bulletin 121(1): 61-71; (see also Nappi, F., Giacinto, O., Lusini, M., Garo, M., Caponio, C., and 5 others 2022. “Patients with Bicuspid Aortopathy and Aortic Dilatation,” Online, MDPI [Multidisciplinary Digital Publishing Institute] Journal of Clinical Medicine (Basel, Switzerland) 11(20): 6002; Rodriguez-Palomares, J. F., Dux-Santoy, L., Guala, A, Kale, R., Maldonado, G., and 10 others 2018. “Aortic Flow Patterns and Wall Shear Stress Maps by 4D-flow Cardiovascular Magnetic Resonance in the Assessment of Aortic Dilatation in Bicuspid Aortic Valve Disease,” Online, Journal of Cardiovascular Magnetic Resonance 20(1): 28). A normal aortic valve tricuspid, if bicuspid, the valve should prompt a close evaluation of the patient for impending aortic dilation and dissection (Avadhani, S. A., Martin-Doyle, W., Shaikh, A. Y., and Pape, L. A. 2015. “Predictors of Ascending Aortic Dilation in Bicuspid Aortic Valve Disease: A Five-year Prospective Study,” American Journal of Medicine 128(6): 647-652).

Progressive dilatation with the possibility of an aneurysm or rupture and the risk of dissection of the thoracic aorta under the considerable hemodynamic stress of left ventricular ejection pressure in the newborn can be prevented by replacing the thoracic aorta as described below in section 2b(2)(c2), entitled Automatic Insertion into Ductus of Inline Coupling Jacket-secured Bridging, or Tie-line. Currently, no conventional repair is satisfactory either in allowing for much growth when the rate of growth in a neonate is considerable, or freedom from becoming defective exists. In this situation, a prosthesis to replace any segment along a congenitally weak and/or malformed major vessel which is structurally durable, nondehisceable, nonrupturable, nondislodgeable, and capable of growth for years if not to the end of life is an effectively dispositive repair.

Ultimately, all conventional forms of repair to the thoracic aorta lack adequate durability—can dehisce, restenose, reaneurysmize, or develop incisions, and afford little if any expandability to accommodate growth—to none of which is a properly designed prosthesis susceptible, and the prosthesis as described below is capable of expanding with growth from birth to adulthood, throughout with a terminal bursting pressure of which no heart is capable. Given that malformations such as an interrupted aortic arch or aneurysm are likely genetic, a lack of repair durability, and/or an inability of the repair to accommodate growth, and/or continued development of the arteries without sufficient strength, especially in the great arteries where the stresses are greater, results in postoperative sequelae is predictable.

Accordingly, in section 2b(2)(f) below, entitled Expandable Structure to Accommodate Growth and Self-adjust in Size in Vascular Segment Prostheses, Lines, and Confluence Conduits Placed in Children, there is described, and in the detailed transectional view indicated in FIG. 36A and provided in FIG. 36D there is shown, a sectional view through a special fabric suitable for use in a prosthesis made to replace a segment along a major vessel such as the aortic arch, the more extended thoracic aorta, or a severely aneurysmal segment along the abdominal aorta. Since a structural failure in such a device would almost certainly result in death, the materials and structure of the prosthesis must meet the highest standards for dependability.

While the need to accommodate growth does not pertain to an abdominal aortic aneurysm usually acquired as an adult, the same type prosthesis is superior to current stent-grafts. Expandable prostheses are injection molded as a seamless tube with or without branches. The prosthesis can have a degree of expandability and durability such that placed in an infant it can serve over the life of the patient. Moreover, pulse compliant, the inline coupling jackets used to secure an expandable prosthesis accommodate druglines to allow the direct targeting to the prosthesis of medication.

The incorporation of sufficient plasticizer precludes a failure of the circumferential expansion vaults which project from the longitudinal axis of the prosthesis to 1. Comply with the additional volume and pressure associated with growth, 2. Not to yield when in contact with neighboring tissue as to abrade it, and 3. Imparts a flexibility that prevents fatigue failure responsive to the pulse. The end failure or breaking tensile strength is greater than a direct blow could overcome. The tie-line, or prosthesis, is secured at either end to the stumps of the excised native vessel by inline coupling jackets which are stitched to the stumps eliminating the risk of migration.

Placed along the aorta with its strong pulse, to prevent disconnection, the inline coupling jackets used may incorporate luminal barbs as in the end-inserted device shown in FIG. 31B and/or grappling hooks that extend from the ends of the jackets to hook into the adventitia of the substrate ductus coupled. An anatomically approximated tie-line to serve as a prosthesis such as that shown in FIG. 36F can replace any segment along or the entire aorta with branches. Each end of the prosthesis is connected to the trunk or branches with inline coupling jackets actuated simultaneously. Repair thus is through open surgery; however, the procedural duration should be less than with conventional open repair and performed under regional anesthesia.

Unlike an endoprosthesis, placement in a child can accommodate growth indefinitely, and the complications encountered with a conventional endoprosthesis such as the development of an endoleak do not apply. Devised to eliminate the sideways protrusion of valves and servovalves as well as a need for the space to accommodate a parallel bypass line where the anatomy affords none, inline coupling jackets have a more linear configuration but one obtained at the expense of the ability to retain the native segment and switch between it and the prosthesis. Whether of the type shown in FIG. 28 or FIG. 32G, where one line is that native and the other prosthetic, to provide a switching junction detracts from lateral clearance.

The ability to switch between the prosthesis and the native structure does, however, provide advantages in allowing flow to be bypassed about the native ductus during healing, and replace it in the event of any cause for the stoppage of flow. Where the native thoracic aorta can be preserved, switching to it can allow normal chemo- and baroreception; however, in practice, this eventuality is elusive and compensation for the loss of this silent, or unfelt, autonomic sensation increases over time.

Compensation for the loss in such sensation following the excision of tumors of the carotid bodies is documented below in section 2b(2)(g), entitled Ischemia and Hypoperfusion-free Carotid Endarterectomy Bypass and Prosthesis. Where repair thus is possible, a. prosthetic disorder response system can incorporate one or more sensors to detect such a condition and initiate switching immediately to preserve perfusion with the interruption only momentary. Loss of either the carotid or aortic bodies is virtually never of both. Endovascular repair which omits the supra-aortic branches can preserve the aortic bodies but is unable to replace the supra-aortic branches dependably. The loss of sensation provided by either the carotid or the aortic bodies is compensated for by the other which is invariably retained.

Exceptionally, inline coupling jackets could be used as valves; however, the ability to preserve the native line and switch between it and a bypass line eliminated, application thus is suitable where the native line is unsalvageable or to retain it dangerous. One suitable example is that given above of the neonate born with a connective tissue disorder and weakened arterial walls susceptible to structural failure that could eventuate in rupture at any time.

Others include a severe coarctation or interrupted aortic arch which unless it lends itself to endovascular aortic repair seldom allows a reconstruction that preserves the aortic bodies (see, for example, Pen, D., Shanks, J., Barrett, C., Abukar, Y., Paton, J. F. R., and Ramchandra, R. 2022. “Aortic Body Chemoreceptors Regulate Coronary Blood Flow in Conscious Control and Hypertensive Sheep,” Hypertension 79(6): 1275-1285; Eugenin, J., Larrain, C., and Zapata, P. 1989. “Correlative Contribution of Carotid and Aortic Afferences to the Ventilatory Chemosensory Drive in Steady-state Normoxia and to the Ventilatory Chemoreflexes Induced by Transient Hypoxia,” (in English), Archivos de Biologia y Medicina Experimentales (Santiago, Chile) 22(4) 395-408).

The availability of a prosthesis for replacing a segment along the thoracic and/or thoracoabdominal or the abdominal aorta without regard to the detailed position and geometry of the defect and available in an expandable type for pediatric use offers advantages to include: 1. Eliminating the need for the geometrical acuity and the time needed to select or alter if not originally design a patch aortoplasty, for example, and 2. By materially reducing the expertise required to undertake such a procedure, makes it possible for general surgeons at more readily accessible community hospitals to accomplish satisfactory repair that would otherwise have required an inordinate degree of knowledge, skill, and ability only found in the largest medical centers.

### Venous Defects

Defects along linear and branched segments of a large vein such as the inferior vena cava are relatively rare but when these do occur, the life of the patient may depend upon a leak-free remedy (see, for example, Truty, M. J. and Bower, T. C. 2007. “Congenital Anomalies of the Inferior Vena Cava and Left Renal Vein: Implications during Open Abdominal Aortic Aneurysm Reconstruction,” Annals of Vascular Surgery 21(2): 186-197). Of the numerous anomalies that appear in veins, few-mostly aneurysms congenital or resulting from chronic hypertension, infection, or injury—are correctable using inline coupling jackets with a bridging, or tie-line as addressed below in the six sections beginning with section 2b(2)(c), entitled Vascular Segment Prostheses: Inline Coupling Jackets and Bridging, or Tie-lines.

When collateral defects are present and not included in the prosthesis, conventional repair is advisable. If stable, a venous aneurysm containing clot is replaced in one step; however, a large fusiform or saccular aneurysm may exceed in diameter a native segment which the automatic tie-line insertion mechanism described below in section 2b(2)(c)(c2), entitled Automatic Insertion into a Ductus of an Inline Coupling Jacket-secured Bridging, or Tie-line, and Its Replacement if Necessary, and shown in FIGS. 37A thru 37E is able to rotate out of position for virtually concurrent replacement (see, for example, Verma, M., Pandey, N. N., Ojha, V., Kumar, S., and Ramakrishnan, S. 2022. “Developmental Anomalies of the Inferior Vena Cava and Its Tributaries: What the Radiologist Needs to Know?,” Online, Indian Journal of Radiology and Imaging 32(3): 355-364; Zuo, Y., Zhang, Z., Shi, B., Ji, Z., and Huang, Z. 2020. “Multiple Inferior Vena Cava Aneurysms Mimic a Retroperitoneal Tumor: A Case Report,” Online, BioMed Central Urology 20(1): 137; Momeni, M. and Momeni F. 2019. “Ruptured Inferior Vena Cava Aneurysm in the Setting of Mural Vascular Malformation: A Case Report,” Journal of Clinical Ultrasound 47(7): 423-425; Tadayon, N., Kalantar-Motamedi, S. M., Zarrintan, S., and Tayyebi, A. 2019. “Isolated Inferior Vena Cava Aneurysm: A Case Report,” Online, Journal of Cardiovascular and Thoracic Research 11(1): 72-74; Inoue, M., Sudo, T., Yamaguchi, M., Seo, S., Miyamoto, T., and 8 others 2018. “Aneurysm of the Inferior Vena Cava with Thrombosis,” Online, Clinical Case Reports 6(2): 402-406).

Defects of the superior vena cava are made evident by superior vena cava syndrome, which denotes various causes for to its obstruction. These include thrombus, fistulation (fistulization), or most often, the formation within the mediastinum or lung of a malignant neoplasm, which often grows into the right atrium. Other causes include a benign neoplasm in the mediastinum, aneurysm of the thoracic aorta, and fibrosing mediastinitis. Symptoms include dyspnea, edema, facial swelling, cyanosis, dysphagia, stridor, cough, engorged vasculature in or discoloration of the head and neck, discoloration of the arms, recurrent syncope, enlargement of the right breast, conjunctival suffusion, and cerebral edema causing brain fog, cephalalgia, and coma.

Thrombus induced superior vena cava syndrome has been dispelled through the administration of oral anticoagulation therapy (Huang, W., Chen, Y., Liu, Z., Huang, Y., and Hu, Y. 2023. “Treatment of Pacemaker-induced Superior Vena Cava Syndrome by Direct Oral Anticoagulant,” Online, BioMed Central Thrombosis Journal 21(1): 19; Fukui, T. and Ogasawara, N. 2022. “Oral Anticoagulation Therapy for Pacemaker Lead-induced Superior Vena Cava Syndrome,” Online, European Heart Journal Case Reports 6(11): ytac433; Mumoli, N., Mazzone, A., Evangelista, I., Cei, M., and Colombo, A. 2021. “Superior Vena Cava Syndrome after Pacemaker Implantation Treated with Direct Oral Anticoagulation,” Online, BioMed Central Thrombosis Journal 19(1): 84).

Where replacement of a segment of the superior vena cava is necessary, the placement of an inline coupling jacketed bridge or tie-line avoids the need for ischemia and cardiopulmonary support. However, where replacement is unnecessary, avoiding the need for an open surgical field through a large entry wound where the anatomy is crowded and space limited should take precedence, making percutaneous transluminal treatment preferable.

Both eventualities are well documented, replacement usually necessitating collateral surgery (see, for example, Arrosi, A. V., Dermawan, J. K., Bolen, M., and Raymond, D. 2022. “Thymomas with Intravascular and Intracardiac Growth,” Online, Frontiers in Oncology 12:881553; Check, L., Naz, A., Scott, C., and Duff, R. 2022. “Endobronchial Ultrasound Diagnosis of a Malignant Superior Vena Cava Tumor Thrombus Extending into the Right Atrium: An Unusual Cause of Recurrent Syncope,” Online, Cureus (Palo Alto, California) 14(11): e31704; Haider, M. S., Master, M. F., Atluri, S., and Nfonoyim, J. 2022. “Superior Vena Cava Syndrome Due to Thrombosis: A Case Report,” Online, Cureus (Palo Alto, California) 14(5): e24811; Seligson, M. T. and Surowiec, S. M. 2022. “Superior Vena Cava Syndrome,” Online, Treasure Island, Florida: StatPearls Publishing Co.; Trivedi, S. B., Ray, C., Chadalavada, S., Makramalla, A., and Kord, A. 2022. “Superior Vena Cava Syndrome: An Update and Literature Review of Percutaneous Endovascular Treatments,” Seminars in Interventional Radiology 39(4): 446-453; Zhang, X., Chen, L., Zhou, W., Wang, Z., Wang, C., Shi, J., and Yao, F. 2022. “Case Report: Complete Resection of Invasive Thymoma Invading the Superior Vena Cava and Right Atrium under Cardiopulmonary Bypass Support,” Online, Frontiers in Oncology 12:1026524; Azizi, A. H., Shafi, I., Shah, N., Rosenfield, K, Schainfeld, R., Sista, A., and Bashir, R. 2020. “Superior Vena Cava Syndrome,” Online, Journal of the American College of Cardiology Cardiovascular Interventions 13(24): 2896-2910; Yang, T., Hui, R., Wu, Q., Tian, J., and Chen, H. 2019. “An Invasive Thymoma Extending into the Superior Vena Cava and Right Atrium,” Online, Annals of Translational Medicine 7(18); 498; Garcia Carretero, R. 2017. “Non-malignant Superior Vena Cava Syndrome in a Patient with Incidental Diagnosis of a Congenital Vascular Defect,” Online, British Medical Journal Case Reports 2017: bcr2016218844; Friedman, T., Quencer, K. B., Kishore, S. A., Winokur, R. S., and Madoff, D. C. 2017. “Malignant Venous Obstruction: Superior Vena Cava Syndrome and Beyond,” Online, Seminars in Interventional Radiology 34(4): 398-408; Cohen, R., Mena, D., Carbajal-Mendoza, R., Matos, N., and Karki, N. 2008. “Superior Vena Cava Syndrome: A Medical Emergency?,” Online, International Journal of Angiology 17(1): 43-46; Saeed, A. I., Schwartz, A. P., and Limsukon, A. 2006. “Superior Vena Cava Syndrome (SVC Syndrome): A Rare Cause of Conjunctival Suffusion, Mt. Sinai Journal of Medicine 73(8): 1082-1085). Nonobstructive impairment generally indicates persistent left superior vena cava, in itself asymptomatic but often associated with congenital heart defects.

Side-entry valves are associated with intercorporeal, or donor-recipient reciprocal cross-circulation during a compound vascular bypass, or sudden switched-organ solid organ heart transplantation, for example, where the ability to switch the flow of blood from the recipient to the donor, or graft, organ as depicted in FIG. 15A is essential. In a sudden compound vascular bypass, or sudden switched-organ transplant, highly damped nonsparking solenoids are used to switch the bloodstream from the defective or diseased organ to and through the donor organ. In metered compound vascular bypass, or sudden switched-organ transplantation, the transfer of the bloodstream between recipient and donor organs is measured with any adverse reaction instantly detected and responded to by the fully implanted prosthetic disorder response system.

As the origin and destination of blood flow, the compound vascular bypass transplantation blood supply and drainage diversion path of the heart, of which the output as depicted in FIG. 15A where the innate or recipient heart is shown to the right, exceptionally outputs both saturated and unsaturated blood in an overall clockwise direction, while that for any other solid organ similarly shown where that native was on the right with saturated, or arterial, blood delivered from above, and unsaturated blood would depart below, would be counterclockwise.

FIGS. 22B and 32F show how the simple switching ability imparted by solenoid operated valves also makes it possible to switch the flow of blood from passing through the native-native line created once the donor and recipient stumps have been anastomosed to passing through synthetic lines that bypass the anastomoses. This capability makes it possible to separately diagnose and/or treat the anastomoses postoperatively without the hindrance of continued blood flow through the anastomoses.

Accessory channels 8 in the upper valve 48 and 8′ in the lower valve 50 shown in FIG. 22B allow medication to be targeted to either or both anastomoses while blood continues to flow uninterrupted through the untreated native line and bypass around the other anastomosis. In FIG. 32F, to accomplish this selective targeting of medication to the single anastomosis while the blood is switched to bypass the anastomosis requires that the upper valve incorporate an accessory channel 8. In FIG. 22B, the reversal in direction of the lower servovalve diversion chutes associated with the graft organ have not been disconnected and rotated—the apparent reversal is simply due rather to rotation or the bloodline in moving from the original to the ending position.

Reciprocally, different treatment can be applied to only the blood bypassing or only that passing through either anastomosis. Switching blood flow to bypass the anastomoses thus allows periodic examination and treatment of the anastomoses alone. The anastomoses can be temporarily relieved of any stress associated with the passage of blood, its pressure, or shear stress, so that absorbable suture can be allowed to dissolve without the risk of dehiscence, for example.

These simple or bistable switching, as opposed to gradual, or metered, switching techniques-sudden compound vascular bypass, or sudden switched-organ transplantation and the ability to pass blood through or to bypass anastomoses—can also be accomplished using the scheme depicted in FIGS. 33A and 33B where instead of both pipelines being synthetic, one of them is organic where the actuator positioned to rotate bistable stopper 175 unseen as on the back side of stopper axle 177 is a miniature rotary solenoid.

For precise synchrony, inlet and outlet solenoids on all switchable lines and/or confluence conduits in use at a given time actuated from a single switch by a member of the surgical team. A backup mechanical key to synchronize the switching between two lines or confluence conduits is shown in FIGS. 34A and 34B Adaptability to gradual or metered switching is then contingent upon the availability of highly miniaturized rotary servovalves. These would ameliorate but not eliminate the problem of sideways protrusion in crowded locations. That such a mechanical key could be replaced by rotary solenoids actuated from the same switch is considered obvious.

In comparison, inline coupling jackets provide a means for end-to-end joining organic and synthetic pipelines with little sideways protrusion but at the cost of omitting switching capability. When a potential need therefor is predicted, accessory channels are made large enough in caliber to admit miniature cabled devices such as fine fiberscopes, excimer lasers, or intravascular ultrasound probes. The use of anastomosis flow-through to bypass switching as just addressed allows the periodic examination and treatment of bypassed anastomoses, for example. These are accessed directly through openings respective of each in a body surface port such as those shown in FIGS. 26A and 26C.

Inline coupling jacket-connected lines are suitable for replacing a diseased, congenitally malformed, or aneurysmal segment along a vessel with a segment-bridging prosthesis where a switching ability that allowed choosing between the passage of blood through the since discarded native or prosthetic segment is uninvolved. Application thus is intracorporeal. In contrast, ductus side-entry devices to include diversion jackets, side-entry valves, and servovalves could be used to connect a prosthetic replacement for a diseased or defective segment of the vessel so that if the native structure-such as following the use of an antibiotic were to heal, the prosthesis could be removed. The possibility is stated to better distinguish between inline coupling jackets and side-entry jackets which not inline, provide valving function. However, the rarity of a need for such application and that a sidestem at right angles to the main axis would likely encroach upon neighboring tissue, it is mentioned as exceptional.

While accessory channels are ordinarily available for the purpose, should one or more of these already be in use to deliver medication, or a future need for a path from a surface port to the objective had not been predictable, an alternate path leading to the objective can be provided. However, without a lead-in pipeline from a surface port, access must be endoscopic through a ‘keyhole’ incision. To this end, where a future need for cable access into a jacket, valve, or servovalve is undeterminable, push-in spring-retum caps over passages leading into the component and the structure it encircles to grasp about or the tissue to which it is connected are provided for the insertion of the distal ends of midprocedural catheteric drug pipelines.

That a vessel segment replacing prosthesis- or any other inline coupling jacket-secured tie-line device—can be made to expand with growth means that for an unwell prenate, neonate, or infant, the need for revision is considerably forestalled, perhaps even eliminated. A prosthesis to replace the aortic arch in an adult without a connective tissue disorder presenting with an aneurysm or in an infant with a less severe coarctation is not recommended. These are best repaired using conventional methods, the aneurysm by excision (aneurysmectomy) and anastomosis (aneurysmoplasty, aneurysmorrhaphy, endoaneurysmor-rhaphy, Matas operation), the coarctation by angioplasty.

However, following relatively localized (focal, in situ) treatment such as an angioplasty or angioplasty and stenting-which in distending the lumen can further weaken the arterial wall-even less extensive defects may restenose, aneurysmize, reaneurysmize, pseudoaneurysmize, or incise. Considering the aneurysm had presented at birth if not in utero, these sequelae indicate that the cause had likely been a malconstitution of the arterial walls during development which resulted in a lack of adequate strength. Congenital connective tissue disorders such as vascular type Ehlers-Danlos syndrome or autosomal recessive cutis laxa and others specified above in this section can result in congenital malformities of the thoracic aorta such as an aneurysm as can Marfan and para-Marfan syndromes, which can cause an irreversible dilatation with the risks of rupture and dissection.

When associated with a malformity of the heart so severe that the prospect of a surgical repair capable of initiating pulsatile flow is not possible, it is preferable to harvest the replacement heart together with the thoracic aorta and its supra-aortic branches for transplantation, preferably using the metered compound vascular bypass technique. Because the different expressions of the disease are due to various defects in the formation of collagen, that the graft organs will undergo similar degeneration following placement in a nonaffected recipient is not a concern.

If seen after infancy or early childhood due to aortic dissection or aneurysm with the threat of rupture, for example, and the patient is additionally affected with intractable cystic lung disease or recurrent pneumothorax, consideration is given to a heart, thoracic aorta, and lung transplant, preferably using the metered compound vascular bypass technique. If for any reason the thoracic aorta of the donor or donors is unfit for a combined transplantation, a metered compound vascular bypass heart transplant is accompanied by the replacement of the thoracic aorta with a prosthesis using the automated procedure for supplanting a segment of a vessel described below.

Due to the greater output pressure into the ascending aorta of a healthy heart, replacement of the heart should follow replacement of the vulnerable thoracic aorta. This same consideration should dictate the concurrent replacement of lungs with interstitial lung disease, common in connective tissue disorders (see, for example, King, T. E. Jr. 2005. “Interstitial Lung Diseases,” in Kasper, D. L, Braunwald, E., Fauci, A. S., Hauser, S. L., Longo, D. L., and Jameson, J. L. Harrison's Principles of Internal Medicine, Chapter 243, New York, New York: McGraw-Hill Medical Publishing Division), should precede replacement of the heart.

While a replacement heart remains unavailable, the use of a continuous flow ventricular assist device as a bridge over more than a relatively short time until a replacement heart becomes available for a neonate, infant, or child is ill-advised as risking the global and irreversible impairments in development associated with the impairment that result from Fontan circulation, or the nonpulsatile blood flow that ensues following the three stages in the repair of a patient with a univentricular, hypoplastic left heart, tricuspid atresia, or double outlet left ventricle, for example. The scarcity of total artificial hearts used as destination rather than bridge therapy as well as common sense attests to the preferability of a real to any mechanical heart.

### Superiority of a Second Heart Over any Mechanical Assist Device

In most of the world, a ventricular assist device, which also provides nonpulsatile blood flow, or a total artificial heart, which does provide pulsatile flow, is unavailable. The view here is that a second heart is superior to any mechanical device anywhere in the world. While at home or at work, the patient can recharge his power storage device by means of transcutaneous energy tranter, most if not all other places will require that he wear a charged power pack. If the double heart transplant imparts circulatory sufficiency, an intention to use it as a bridge rather than to accept it as the destination treatment can be abandoned, eliminating further surgical trauma. Furthermore, the usability of a less than perfect graft heart substantially increases the pool of available hearts.

A compound vascular bypass double heart transplant, as described below in section 2b(1)(b)(b5), entitled Heterotopic Double Heart Metered Compound Vascular Bypass, or Switched-organ Transplantation [and so on], is preferable, because of: 1. The considerably greater pool of adequate graft organs, 2. The graft is free of charge, 3. The relative simplicity of the operation compared to the conventional three-stage Norwood, Glenn or Hemi-Fontan, and Fontan procedures which are intricate and 4. Cannot be undertaken by the more numerous accessible general surgeons in a local hospital and not just pediatric cardiac transplantation specialists in major medical centers, 5. The fact that neither the native nor the graft heart is ever traumatized through extensive incisions and ischemia, 6. The fact that the patient need not be placed on cardiopulmonary bypass life support and general anesthesia lasting several hours, 7. The second heart as an assist device provides pulsatile, not continuous flow. 8. Unlike most ventricular assist devices and total artificial hearts, a second heart does not require a sizable paracorporeal power pack which the patient must constantly wear and can be a considerable impediment and degradation in the quality of life, 9. A mechanical device cannot discharge the number of movements required to beat as would a heart over years, and nonpulsatile flow is known to be injurious, 10. A total artificial heart generally weighs about three times as much as an equivalent real heart. 11. The added heart need not match that native in size; allowing the use of a smaller heart with less of a demand for space and placement in the chest as preferred. 12. Absent rejection, the added heart is compatible with the physiology presented by its new milieu, and 13. Its cells generate normal warmth, not excessive heat associated with the inducement of clotting. 14. Unlike mechanical devices, a real heart does not damage platelets which leads to problems of bleeding and clotting, 15. That both the donor and recipient hearts remain intact throughout introduces a factor of graft organ size liberalization, increasing the pool of available hearts, 16. Proceeding directly to the state of circulatory rectification, the first operation is direct-to-destination, not bridge therapy, hence, eliminates a preparatory sequence of operations each posing considerable risk, traumatizing then retraumatizing the patient, to conclusively terminate the damage caused by abnormal circulation, 17. Orthotopic replacement of a failing heart with a ventricular assist device or a total artificial heart with a real heart renders defects in the native heart such as an aortic valve disorder moot, and 18. A real heart grows with the baby; a mechanical device cannot do this and probably never will.

Some of these factors pertain to a conventional heterotopic heart transplant as well whereby rather than to replace the recipient native ventricles, the ventricles of the graft organ are added to those of the recipient. The key distinctions between conventional organ transplants and heart transplants in particular, that the surgical trauma imposed on both donor and recipient hearts, the extended use of cardiopulmonary bypass and general anesthesia, and the ischemia of the graft organ which together with the intimate and stark immune confrontation being directly stitched together almost certainly predispose the transplant to accelerated deterioration, are much the same in a conventional heterotopic as in a conventional orthotopic heart transplants (see, for example, Kummer, L., Zaradzki. M., Vijayan, V., Aril. R., Weigand, M. A., and 3 others 2020, Op cit., above in this section; Cross. A. R., Glotz, D., and Mooney, N. 2018. “The Role of the Endothelium during Antibody-mediated Rejection: From Victim to Accomplice,” Online, Frontiers in Immunology (Lausanne, Switzerland) 9:106).

Because a compound vascular bypass heterotopic heart transplant leaves both recipient and donor hearts separate and distinct, the donor heart can be smaller in size, which facilitates positioning it in different locations to minimize or even avoid encroachment upon neighboring organs and tissues. The hearts can be diagnosed and treated separately, and should the native heart recover, that added be rejected, or the native heart completely fail, either can be removed, and if necessary, replaced—an eventuality that a conventional heterotopic transplant decidedly rules out. The latter can be replaced, but not without the surgical trauma and extended cardiopulmonary bypass and general anesthesia that will predispose the replacement to deterioration no differently than the initial operation.

An unanswered question is whether following the procedure specified in below in section 2b(1)(b)(b5)(see section 2b(1)(b)(b5) below entitled Heterotopic Double Heart Metered Compound Vascular Bypass, or Switched-organ Transplantation [and so on]), which calls for resecting a lower portion of the right lung so that it will regenerate within its reduced space with a compensatory higher density of alveoli to allow placement of the added heart in the chest as preferred will pose unanticipated complications (see section 2b(1)(b)(b5), entitled Heterotopic Double Heart Metered Compound Vascular Bypass, or Switched-organ transplantation [and so on]. However, the reduced lung should recover to complete functionality, and this preliminary step will overcome the atelectasis caused by compression of the lower two lobes caused by the donor ventricles in a conventional heterotopic heart transplant (see, for example, Vaijyanath, P. 2020. “Heterotopic Heart Transplant: Relevance as Bio-VAD in Emerging Economies,” Online, Indian Journal of Thoracic and Cardiovascular Surgery (New Delhi, India) 36 (Supplement 2): 279-286). Creating sufficient space for the donor ventricles in a conventional heterotopic heart transplant by removing a portion of the lower right lung might have done away with this complication but this measure appears not to have been employed (see, for example, Cockrell, H. C., O'Brien, R., Carter, K. T., Shaw, T. B., Baran, D. A., and 3 others 2021, Op cit., above in this section;

Furthermore, a conventional heterotopic heart transplant is susceptible to serious complications which usually overcome, are not so without considerable anxiety for the patient (see, for example, Kealaher, E., Edwards, H., Wheeler, R., Zaidi, A., and Sharp, A. S. P. 2021. “Now You See It . . . : When Lead Positions Tell 2 Stories,” Online, Journal of the American College of Cardiology. Case Reports (Amsterdam, North Holland, Netherlands) 3(11): 1398-1399; Tagusari. O., Kormos, R. L., Kawai, A., Yamazaki, K., Pham, S. M. and 3 others 1999. “Native Heart Complications after Heterotopic Heart Transplantation: Insight into the Potential Risk of Left Ventricular Assist Device,” The Journal of Heart and Lung Transplantation (New York, New York) 18(11): 1111-1119; Konertz, W., Sheikhzadeh, A., Weyand, M., Friedl. A., and Bernhard, A. 1988. “Heterotopic Heart Transplantation: Current Indications for the Procedure, with Results in 10 Patients,” Texas Heart Institute Journal 15(3): 159-162).

In a neonate, infant, or older pediatric patient, the facts that unlike a real heart, a mechanical assist device or a total artificial heart cannot grow with the child, and that even in an adult, a mechanical device is rarely opted for as the destination therapy, replacement of the innate with a sound heart, or if none is available, then the placement of a second heart as described below in section 2b(1)(b)(b5), entitled Heterotopic Double Heart Metered Compound Vascular Bypass, or Switched-organ Transplantation [and so on], is not just superior from a technical standpoint, but because it will grow as the destination therapy, the patient will be spared numerous complications associated with and following at least one more major operation.

The superiority of a metered compound vascular bypass orthotopic or heterotopic heart transplant in an adult with end stage heart failure is likewise destination therapy with the only mechanism known now and probably forever that will prove capable of discharging the full functionality of a real heart, and that is another real heart. The same must be said for surgical reconstructions which seldom if ever accomplish more than to save the life of the baby as important as that is, but always with an outcome that is devastating from the standpoint of day-to-day well-being and potential. Unlike mechanical devices which are seldom other than a temporary half-way measure; the immediate replacement of the defective heart with a sound heart or an adequate second heart can preclude the alternative sequence of events.

To provide a replacement heart is the solution most consistent with normal function and development. Furthermore, because the metered compound vascular bypass method for organ transplantation eliminates both ischemia and surgical trauma for both the donor and recipient hearts, it is incontestably superior to the current practice in eliminating the factors in a conventional heart transplant that are strongly suspected to eventuate in cardiac allograft vasculopathy and/or cancer. Finally, having administered the transplantation operation, the fully implanted disorder response system continues postoperatively to dispel what would otherwise have been a lifelong apprehension for complications.

These complications include susceptibility to infections, arrhythmias, and rejection, which constant concerns the system dispels by automatically dispensing the counteractant drugs. Ending the anxiety over forgetting to take the medication prescribed, which could prove catastrophic, alone represents a considerable improvement in the patient's qualify of life. That the control system can immediately detect and dispel these eventualities brings the patient to one step short of having been born without a defective heart.

Whether due to postoperative Fontan circulation or a ventricular assist device, with time, the impact of long-term nonpulsatile flow during the development of a child becomes more and more harmful, the extended use of such a device leading to bleeding problems, for example. No total artificial heart has been approved as a destination treatment. Use of a continuous flow left ventricular device does not necessarily completely mask an underlying pulsation (Slaughter 2010 cited below); however, an inadequate level of pulsatile intensity is probably insufficient to counter the adverse consequences of what is essentially nonpulsatile flow.

In a compound vascular bypass heart transplant to replace a heart at end stage heart failure or the severely malformed heart in a prenate, neonate, or toddler, use of a positive inotrope should be necessary only until transfer of the graft organ into the recipient has been completed, and then, likely only in the longer bloodlines or confluence conduits in switchable long to short or the reverse switchable lines or conduits. Even in an extended metered transplant, the duration should be too slight for the use of an inotrope to prove problematic.

Otherwise, the use of positive inotropes carries numerous risks to include deviations in blood pressure, cephalalgia, anxiety, tachycardia, myocardial oxygen consumption leading to arrhythmia and myocardial ischemia, and fever. Indeed, the risks encountered with the long-term use of positive inotropes and vasopressors might be sufficient in itself to justify a compound vascular bypass heart transplant (see for example, Ahmad T. Miller P E, Mccullough M, Desai N R, Riello R. and 6 others 2019. “Why Has Positive Inotropy Failed in Chronic Heart Failure? Lessons from Prior Inotrope Trials,” Online, European Journal of Heart Failure (Chichester, England) 21(9): 1064-1078; Francis, G. S., Bartos, J. A., and Adatya, S. 2014. “Inotropes,” Online, Journal of the American College of Cardiology (New York, New York) 63(20): 2069-2078)(see also, for example, Agakidou, E., Chatziioannidis, I., Kontou, A., Stathopoulou, T., Chotas, W., and Sarafidis, K. 2024. “An Update on Pharmacologic Management of Neonatal Hypotension: When, Why, and Which Medication,” Online, MDPI [Multidisciplinary Digital Publishing Institute] Children (Basel, Switzerland) 11(4): 490; Ramaswamy VV. Kumar G, Abdul Kareem P. Somasekhara Aradhya A, Suryawanshi P. and 4 others 2024. “Comparative Efficacy of Volume Expansion, Inotropes and Vasopressors in Preterm Neonates with Probable Transitional Circulatory Instability in the First Week of Life: A Systematic Review and Network Meta-analysis,” Online, British Medical Journal Paediatrics Open (London, England) 8(1): e002500; Acharya, D., Kazui. T., Al Rameni. D., Acharya, T., Betterton, and 5 others 2023. “Aortic Valve Disorders and Left Ventricular Assist Devices,” Online, Frontiers in Cardiovascular Medicine (Lausanne, Switzerland) 10:1098348; Baronos, S., Whitford, R. C., and Adkins, K. 2023. “Postoperative Care after Left Ventricular Assist Device Implantation: Considerations for the Cardiac Surgical Intensivist,” Indian Journal of Thoracic and Cardiovascular Surgery (New Delhi) 39 (Suppl 1): 182-189; Hammer, Y., Bitar, A., and Aaronson, K. D. 2023. “Gastrointestinal Bleeding on Continuous-flow Left Ventricular Assist Device Therapy,” Online, ESC [European Society of Cardiology] Heart Failure (Oxford, England) 2023 10(4): 2214-2224; Kataoka, N. and Imamura, T. 2023. “Catheter Ablation for Tachyarrhythmias in Left Ventricular Assist Device Recipients: Clinical Significance and Technical Tips,” Online, MDPI [Multidisciplinary Digital Publishing Institute] Journal of Clinical Medicine (Basel, Switzerland) 12(22): 7111; Liesdek, O. C. D., Schutgens, R. E. G., de Heer, L. M., Ramjankhan, F. Z., Sebastian, S. A. E., and 3 others 2023. “Acquired Blood Platelet Disorder in Patients with End-stage Heart Failure after Implantation of a Continuous Centrifugal-flow Left Ventricular Assist Device: A Prospective Cohort Study,” Online, Research and Practice in Thrombosis and Haemostasis (New York, New York) 7(2): 100101; Roka-Moiia, Y., Ammann, K. R., Miller-Gutierrez, S., Sheriff. J., Bluestein, D., and 3 others 2023. “Shear-mediated Platelet Microparticles Demonstrate Phenotypic Heterogeneity as to Morphology, Receptor Distribution, and Hemostatic Function,” Online, MDPI [Multidisciplinary Digital Publishing Institute] International Journal of Molecular Sciences (Basel, Switzerland) 24(8): 7386; Shankar, A., Gurumurthy, G., Sridharan, L., Gupta, D., Nicholson, W. J., Jaber, W. A., and Vallabhajosyula, S. 2022. “A Clinical Update on Vasoactive Medication in the Management of Cardiogenic Shock,” Online, Clinical Medicine Insights. Cardiology (Thousand Oaks, California) 16:11795468221075064; Calin, E., Ducharme, A., Carrier, M., Lamarche, Y., Ben Ali, W., and Noly, P. E. 2022. “Key Questions about Aortic Insufficiency in Patients with Durable Left Ventricular Assist Devices,” Online, Frontiers in Cardiovascular Medicine (Lausanne, Switzerland) 9:1068707; Chaudhry S P. DeVore A D. Vidula H. Nassif M. Mudy K. and 7 others 2022. “Left Ventricular Assist Devices: A Primer for the General Cardiologist,” Journal of the American Heart Association (Oxford, England) 11(24): e027251; Pasha, A. K., Lee, J. Z., Desai, H., Hashemzadeh, M., and Movahed, M. R. 2022. “In-hospital Complications Associated with Total Artificial Heart Implantation in the United States between 2004 to 2011,” Online, American Journal of Cardiovascular Disease (Madison, Wisconsin) 12(5): 278-282; MeNamara, N., Narroway, H., Williams, M., Brookes. J., Farag. J., and 5 others 2021. “Contemporary Outcomes of Continuous-Flow Left Ventricular Assist Devices—A Systematic Review,” Online, Annals of Cardiothoracic Surgery (Hong Kong, China) 10(2): 186-208; Stem, B., Maheshwari. P., Gorrepati. V. S., Bethards, D., Chintanaboina, J., Boehmer, J., and Clarke, K. 2021. “Initial Endoscopic Intervention is Not Associated with Reduced Risk of Recurrent Gastrointestinal Bleeding in Left Ventricular Assist Device Patients,” Annals of Gastroenterology (Athens, Greece) 34(5): 660-668; Besser, M. W. 2020. “Post-operative of Bleeding, Haemolysis and Coagulation in Mechanical Circulatory Support Patients, Online, Annals of Translational Medicine (Hong Kong, China) 8(13): 832; Hockstein, M. A., 2020. “Continuous-flow Left Ventricular Assist Devices: Management in the Emergency Department,” Online, Journal of the American College of Emergency Physicians Open (Hoboken, N. J.) 1(4): 362-370; Apostoli, A., Bianchi, V., Bono, N., Dimasi. A., Ammann, K. R. and 10 others 2019. “Prothrombotic Activity of Cytokine-activated Endothelial Cells and Shear-activated Platelets in the Setting of Ventricular Assist Device Support,” Online, Journal of Heart and Lung Transplantation (New York, New York) 38(6): 658-667; Z., Zhang, J., Kareem, K., Tran, D., Conway, R. G., and 3 others 2019. “Device-induced Platelet Dysfunction in Mechanically Assisted Circulation Increases the Risks of Thrombosis and Bleeding,” Online, Artificial Organs (Cambridge, Massachusetts) 43(8): 745-755; Kadado, A. J. Akar, J. G., and Hummel, J. P. 2018. “Arrhythmias after Left Ventricular Assist Device Implantation: Incidence and Management,” Online, Trends in Cardiovascular Medicine (New York, New York) 28(1): 41-50; Pathak, V., Donovan, C., and Malhotra, R. 2017. “Airway Complications of Total Artificial Heart,” Online, Indian Journal of Critical Care Medicine (New Delhi, India) 21(2): 94-95; Amado, J., Gago, P., Santos, W., Mimoso, J., and de Jesus, I. 2016. “Cardiogenic Shock: Inotropes and Vasopressors,” (in English and Portuguese), Online, Revista Portuguesa de Cardiologia (Lisbon, Portugal) 35(12): 681-695; Capoccia, M. 2016. “Mechanical Circulatory Support for Advanced Heart Failure: Are We about to Witness a New “Gold Standard?,” Online, MDPI [Multidisciplinary Digital Publishing Institute] Journal of Cardiovascular Development and Disease (Basel, Switzerland) 3(3): 35; Yost, G., Joseph, C. R., Royston, T., Tatooles, A., and Bhat, G. 2016. “Heat Generation in Axial and Centrifugal Flow Left Ventricular Assist Devices,” Online, American Society for Artificial Internal Organs 62(6): 684-689; Cook, J. A., Shah, K. B., Quader, M. A., Cooke, R. H., Kasirajan, V., and 4 others 2015. “The Total Artificial Heart,” Online, Journal of Thoracic Disease (Hong Kong, China) 7(12): 2172-3180; Newsom, L. C. and Paciullo, C. A. 2013. “Coagulation and Complications of Left Ventricular Assist Device Therapy: A Primer for Emergency Nurses,” Online, Advanced Emergency Nursing Journal (Philadelphia, Pennsylvania) 35(4): 293-301-302: Wever-Pinzon, O., Selzman, C. H., Drakos, S. G., Saidi, A., Stoddard, G. J., and 8 others 2013. “Pulsatility and the Risk of Nonsurgical Bleeding in Patients Supported with the Continuous-flow Left Ventricular Assist Device HeartMate II,” Online, Circulation. Heart Failure (Hagerstown, Maryland) 6(3): 517-526; Slaughter, M. S. 2010. ““Long-term Continuous Flow Left Ventricular Assist Device Support and End-organ Function: Prospects for Destination Therapy,” Journal of Cardiac Surgery (Hoboken, N. J.) 25(4): 490-494; Kluckow, M. 2005. “Low Systemic Blood Flow and Pathophysiology of the Preterm Transitional Circulation,” Early Human Development (Limerick, Ireland) 81(5): 429-437; additional references cited below in section 2b(1)(b)(b3), entitled Neonatal and Infant Compound Vascular Bypass, or Switched-organ, Transplantation of the Heart. Mechanical circulatory assist devices are also addressed below in this and subsequent sections with additional references cited.

A serious vasculitis. Takayasu's arteritis (occlusive thromboaortopathy, aortic arch syndrome, pulseless disease, nonspecific aortoarteritis) may become quite extensive, affecting not just the aortic arch as is more common, but the thoracoabdominal aorta, and numerous branches of the aorta, and the pulmonary artery, placing much of the arterial tree under threat of thrombosis (see, for example, The Merck Manual, 2006, Whitehouse Station, New Jersey: Merck Research Laboratories Division, Merck and Company, Inc., chapter 33, pages 279, 280).

FIG. 36F shows a thoracic aortic neoarch together with its supra-aortic branches to the head and thus represents a simpler case of extension of the prosthesis to branches. The various degenerative changes and risks to luminal patency secondary to the essential disease can be foiled through the placement of a prosthesis that replaces the affected vasculature however extensive. Combined with a totally implanted automatic disorder response system to automatically pipeline-target medication to accessory channels positioned along the prosthesis according to the prescription-program, the many sequelae can be foiled.

Use of a prosthetic aortic arch, or neoarch, for example, described below in section 2b(2)(f), entitled Expandable Structure to Accommodate Growth and Self-adjust in Size in Vascular Segment Prostheses, Lines, and Confluence Conduits Placed in Children, is intended to eliminate a large, life-threatening, defect and/or a defect which to deal with surgically would pose considerable risk and be left susceptible to numerous postoperative complications by expanding with growth at least into adolescence. However, other conditions will justify replacement of an extended segment.

These include a complete interruption or a coarctation bordering on such, a connective tissue disorder with the de facto presentation of an aneurysm, and the permanent repair of a defect otherwise intractable to durable correction. When a problem of recurrence in a life-threatening defect is present in an infant, the method of correction must additionally hold despite growth, something a stent used to sustain an angioplastied coarctation, for example, can sustain only for a time.

The lack of secretory, such as endothelial, and sensory, such as baroreceptor function are limitations of a prosthetic neoarch, for example, which if it would be of significant benefit, can be simulated through the direct targeting of nitrates, for example, and the incorporation of a thin film pressure sensor of which the feedback is used to release remedial medication, for example. By the same token, synthetics succeed well in simulating normal and durable structure; in alleviating the requirement to assure an adequate and dependable blood supply and drainage, in defying disease and every other vulnerability associated with living tissue and their effects, to include infection, ulceration, atherosclerotic degradation, malignancy, and so on, even congenitally defective connective tissue which malacotic, is prone to aneurysm.

Just as do basic ductus side-entry jackets, valves, and servovalves, inline coupling jacket-connected fluid line segment inserts incorporate at least one accessory channel for the delivery of maintenance agents into the jacket and medication into the ductus it encircles to grasp about. As described and illustrated in section 2b(2)(c)(c3) entitled Types of Inline Coupling Jacket-secured Bridging, or Tie-lines, diseased or missing ductus segment replacing bridges, or tie-lines, include thoracic and thoracoabdominal aortic prostheses and those mounting flow diverters, vascular valves and servovalves.

In a fully implanted automatic disorder response system, the microcontroller- or in comorbid disease, the master control microprocessor-governs the direct pipeline-targeting of drugs when the mainline is used to pass blood through the service or accessory channel or channels as directed by the controller on the basis of data received from implanted sensors incorporated into or positioned in relation to the jacket. The controller then commands the release of medication specified in its prescription-program.

The severe consequences of abnormal circulation during pre- and postnatal development of the heart, kidneys, and liver, as well as along the vessels leading from or to the heart and/or other major organs, much less when combined, results in irreversible cognitive and metabolic deficits which the plasticity of nerve notwithstanding, will not only persist but worsen with time and will end in an early death. At birth, a routine checklist of preventive measures are applied to preclude complications such as infection, and the steps prescribed for a physical examination can detect a large number of disorders for which effective treatment has been established.

It now possible to temporarily remove the fetus from the uterus for treatment and return it, once detected, numerous malformities of the viscera confirmed by imaging that reveals a defect such as incomplete closure of the neural groove, for example, can be corrected surgically. Appearing during the prenatal period, irreversible maldevelopment of the major organs and/or other viscera as well as numerous chemical imbalances detectable in utero should be sought out not following birth but rather by earlier interdiction in utero. The number of potential congenital defects unrestrained, relatively very few can be mentioned.

Inborn errors of metabolism due to mutations in genes that code for enzymes picked up in screening blood and urine panels, such as phenylketonuria and galactosemia, as well as endocrine problems, such as hypothyroidism (hypothyrea, hypothyrosis, hypothyreosis hypothyroidea) and adrenal hyperplasia can be suppressed medicinally. Orthopedic defects such as kyphosis and scoliosis are ameliorable, and prostheses can be prepared to offset the effect of missing or irreparable limbs or joints. But treatment for other serious congenital conditions remains inadequate of impossible.

Among those irrevocable are defects of the brain for which only the associated craniofacial malformity can be ameliorated, defects in sensory organs such as result in the absence of hearing or vision which defy surgical reversal, although even severely impaired hearing can be mitigated with cochlear implants, for example, to preserve the ability to communicate, hence, socialize by voice, and defects in vision can often be lessened. The most serious developmental defects result in the death of the fetus, and these are closely followed in severity by conditions that do not kill but preclude the baby from attaining longevity and self-sufficiency.

Of necessity, errors in the treatment of serious heart and related defects, often associated with if not caused by defects in other major organs, typically the lungs, liver, and kidneys, were developed before the advent of immunosuppressive drugs. Only then did organ transplantation appear not just experimental and futile but practicable. Absent organ transplantation as a practical alternative that could materially if not completely discontinue the development of adverse sequelae, the studious collection of information which would have made it clear that the repair had succeeded in keeping the baby alive but not in establishing normal oxygenation and pulsation essential for normal development would have served no purpose.

Now that transplantation has become less contingent, the failure to periodically confirm that the postoperative development of the baby following a heart repair had not established normal circulation is still the norm. If neurological, neuro-ophthalmological, kinesiological, and other physiological performance levels continue to decline relative to the norm, this pattern should not be “left well enough alone” but rather stopped as soon as a replacement heart or other pertinent organ or organs become available: the standard of care to be striven for should not be limited to little more than keeping the infant alive, but rather to eradicate the pattern of progressive decline.

This disposition should have been assumed before birth. Defects of the heart and other organs so severe that to establish normal function through reconstruction using autologous tissue would be impossible should indeed be repaired, but with a view to promptly seize upon a replacement organ. A defect of a major vessel that would prevent normal development should be replaced with a prosthesis capable of expanding in step with growth for years well past the time during which the baby had been too frail to undergo a revision. To this end, described here are fundamentally superior methods for replacing solid organs and incurably defective segments of blood vessels. Both have been devised to maximize simplicity and avoid the need to:

1. Bypass and interrupt innate cardiopulmonary function through the use of conventional cardiopulmonary bypass of the heart and lungs but rather by sustainment of full body circulation through the use of electrical stimulation and pharmacy unless the heart and or lungs must be harvested first with the use of extracorporeal membrane oxygenation. Alternative methods include the use of a pacemaker-defibrillator, with spontaneous breathing sustained with a number of devices and techniques specified below. No means for sustaining circulation and spontaneous breathing are without the risk of adverse sequelae.

2. Intensively monitor a large number of indicia throughout the procedure that require a perfusionist and anesthesiologist with experience and expertise not likely to be present at a small hospital. Transplantation, major vessel replacement, and the prescribed procedures for response to unwanted incidents that might eventuate during either type procedure are largely automated and simplified to the point that a local general surgeon can undertake either under emergency conditions, and can do so with less preparation and less experienced assistants. Organ transplantation has been devised to additionally avoid the need to:

3. Allow the graft organ to undergo the trauma associated with death and the deterioration associated with storage. Instead, brain death imminent, the donor is prepared for sustainment through the use of electrical stimulation and pharmacy. If the heart and/or lungs are needed first, then sustainment in a substantially living condition is accomplished with the aid of extracorporeal membrane oxygenation. Either way, the prospective graft organs continue to be perfused until harvested for direct transfer from the circulatory system of the donor into that of the recipient. Several other means for oxygenating the body are mention below. In an emergency, a sudden transfer, or switched-organ, organ transplant of the graft organ from the circulatory system of the donor into the circulatory system of the recipient is accomplished with the aid of binary, that is, fully open or fully closed, vascular valves.

Otherwise, to accomplish a measure of immune tolerance induction as well as to allow the automatic response system to react to indications of rejection, it is preferred that the operation be carried out using the metered method, meaning gradually with the aid of continuously adjustable vascular servovalves. Nevertheless, because the practical reality is that the donor will often have died when and where life support through the placement of a pacemaker-defibrillator could not have been instituted prior to death, the ex vivo perfusion of a graft organ harvested after the donor died and was similarly transferred directly from the donor to the perfusion machine before transport, while not preferred, must be recognized as necessary (see, for example, Karimian, N. and Yeh, H. 2017 “Opportunities for Therapeutic Intervention during Machine Perfusion,” Current Transplantation Reports 4(2): 141-148; Berendsen, T. A., Bruinsma, B. G., Lee, J., D'Andrea, V., Liu, Q., and 3 others 2012. “A Simplified Subnormothermic Machine Perfusion System Restores Ischemically Damaged Liver Grafts in a Rat Model of Orthotopic Liver Transplantation,” Transplantation Research 1(1): 6).

4. Operate on and thus severely injure both graft and native organs as is essential in conventional orthotopic and heterotopic heart transplantation to effect their union. In contrast, compound vascular bypass, or sudden switched-organ transplantation, orthotopic and heterotopic, or double heart, transplantation, omits the need for directly operating on either the donor graft or recipient organs, which instead remain intact, unincised, throughout either procedure. Mortality pending the availability of a new heart 12 to 15 percent in the United States (The Merck Manual, 2006, 18th edition, ch/pp.), a switch double heart heterotopic transplant allows a native heart undergoing progressive failure to be assisted through the addition of a second heart in a better but not necessarily perfect condition, thus increasing the pool of usable hearts.

Moreover, both procedures take advantage of a totally implanted, or closed-skin automatically controlled disorder response system previously described in U.S. Pat. No. 11,013,858, entitled Nonjacketing Side-entry Connectors and Prosthetic Disorder Response Systems, and U.S. Pat. No. 11,759,186, entitled Ductus Side-entry Jackets and Prosthetic Disorder Response Systems, as well as several sections to follow. While organ transplantation and vascular surgery are ordinarily complex and taxing, such a system complements the advances made in the use of immunosuppressives and other drugs by monitoring and automatically responding to the large number of midprocedural contingencies that might arise during the procedure by directly targeting such drugs in locally, or topically effective (liver metabolized) form, for example, to the trouble spot.

Such a system, described in this and accompanying applications specified in the section above entitled Cross Reference to Related Applications and others, is designed to allow years of growth, even in a neonate. Having automatically administered a switch, or bypass, organ transplant, the implanted system, consisting of a control microprocessor- or in a more elaborate system to treat comorbid conditions as well, a microprocessor-sensors, drug reservoirs ordinarily positioned subcutaneously in the pectoral region, drug reservoir outlet motors, catheteric lines from the reservoir outlets through the connecting jackets for delivery of drugs directly into the target segment of the lumen, continues its prescription-program by responding to site sensor inputs with medication or electrical stimulation, for example.

For immediate response to any adverse sequel, the system should remain permanently implanted in an organ recipient. In an infant, bloodlines are configured as shown in FIGS. 15C, 15D, 36A, and 36F to expand with growth, thus allowing for lifelong service without the need for later revision. In children, any electrical lines associated with the implanted prosthetic disorder response system are either coiled to allow expansion or replaced through radio remote control, the latter also applicable where electrical wiring is difficult to route without the risk of strangulating intervening tissue, although coiling much reduces such an eventuality. Where necessary, system control is by radio, such as Bluetooth, the recharging of rechargeable batteries-usually in the form of tiny button cells—by transcutaneous energy transfer.

The development of implants with inmate or transcutaneous energy transferred power is currently concentrated on the development of implants for optogenetic applications, hardly limited to the precise stimulation of neurons, addressed here and below in this section, as well as other sections, such as 2b (1)(b), entitled Metered Compound Vascular Bypass, or Metered Switched-organ Transplantation, where the reinstatement if not the sustainment of normal synchronization in a heart transplant is imperative. Means to allow optogenetic measures to be applied inside the human body will be directly relevant to the use of fully implanted prosthetic disorder response systems. Such a system can integrate the use of this with other therapeutic means to include the directly pipe-targeted delivery of medication to any given treatment site as scheduled by the prescription-program.

Transgenic treatment of a human patient or even a limited volume of tissue or organ unacceptable, a transgenically prepared optogenetically active agent responsive to photostimulation, typically light gated cation channel Channelrhodopsin-2, is injected or infused into the tissue to be treated by means of optical stimulation-something the implanted disorder control system can accomplish along with the control of the implant-automatically as scheduled by the prescription-program.

Medication for use in conjunction with optogenetic implants can include adverse immune reaction counteractives without which the use of such means could remain without acceptance (see, for example, Kim, C. Y., Ku, M. J., Qazi, R., Nam, H. J., Park, J. W., and 7 others 2021. “Soft Subdermal Implant Capable of Wireless Battery Charging and Programmable Controls for Applications in Optogenetics,” Nature Communications Online at https://doi, org/10.1038/s41467-020-20803-y; Madrid, M. K., Brennan, J. A., Yin, R. T., Knight, H. S., and Efimov, I. R. 2021. “Advances in Implantable Optogenetic Technology for Cardiovascular Research and Medicine,” Online, Frontiers in Physiology 12: 720190;-Mondello, S. E., Pedigo, B. D., Sunshine, M. D., Fischedick, A. E., Homer, P. J., and Moritz, C. T. 2021. “A Micro-LED Implant and Technique for Optogenetic Stimulation of the Rat Spinal Cord,” Online, Experimental Neurology 335:113480; Burton, A., Obaid, S. N., Vázquez-Guardado, A., Schmit, M. B., Stuart, T., and 9 others 2020. “Wireless, Battery-free Subdermally Implantable Photometry Systems for Chronic Recording of Neural Dynamics,” Online, Proceedings of the National Academy of Sciences of the United States of America 117(6): 2835-2845; Liu, C., Zou, G., Feng, M., Sun, Q., Yin, L., Sheng, X., and 6 others 2020. “A Wireless, Implantable Optoelectrochemical Probe for Optogenetic Stimulation and Dopamine Detection,” Microsystems and Nanoengineering 6:64; Qazi, R., Gomez, A. M., Castro, D. C., Zou, Z., Sim, J. Y., and 11 others 2019. “Wireless Optofluidic Brain Probes for Chronic Neuropharmacology and Photostimulation,” Nature Biomedical Engineering 3(8): 655-669; Qazi, R., Kim, C Y., Byun, S.-H., and Jeong, J.-W. 2018. “Microscale Inorganic LED Based Wireless Neural Systems for Chronic in Vivo Optogenetics,” Online, Frontiers in Neuroscience 12:764; McCall, J. G., Qazi, R., Shin, R. G., Li, S., Ikram, M. H., and 6 others 2017. “Preparation and Implementation of Optofluidic Neural Probes for in Vivo Wireless Pharmacology and Optogenetics,” Nature Protocols 12(2): 219-237; Samineni, V. K., Yoon, J., Crawford, K. E., Jeong, Y. R., Mckenzie, K. C., and 16 other 2017. “Fully Implantable, Battery-free Wireless Optoelectronic Devices for Spinal Optogenetics,” Online, Pain 158(11): 2108-2116; Park, S. I. Shin, G., Banks, A., McCall, J. G., Siuda, E. R., and 7 others 2015. “Ultraminiaturized Photovoltaic and Radio Frequency Powered Optoelectronic Systems for Wireless Optogenetics,” Journal of Neural Engineering 12(5): 056002).

The recent development of bioelectronic electrodes for optical implementation which overcome the obstacle of bioelectrodes and implantation methods which “require the tissue to adapt to the electrodes rather than the other way around,” should eliminate complications is addressed below in the final paragraph to the section.

While limited to the clinic, an alternative approach eliminates the need for an optogenetic implant. Instead, a transgenically prepared optogenetically active agent responsive to photostimulation is injected into the subject (Benevides, E. S., Sunchine, M. D., Rana, S., and Fuller, D. D. 2022. “Optogenetic Activation of the Diaphragm, Online, Nature Research Scientific Reports 12(1): 6503). Here the relevance to an implanted disorder response system is that such a system could accomplish the infusion or injection of the photostimulable substance. The tongue is immediately accessible as to negate the need for an implant but the concept of using an injectant pertains no less (Singer, M. L., Benevides, E. S. Rana, S., Sunshine, M. D., Martines, R. C., and 3 others 2023. “Optogenetic Activation of the Tongue in Spontaneously Breathing Mice,” Respiratory Physiology and Neurobiology 309:103998).

The longitudinally separate semicylindrical halves of side-entry jackets are joined by spring-loaded hinges which open in response to growth once the foam internal thereto has already complied to the extent it can, so that to continue the circumferential expansion vaults 234 and flats 233-alternating pattern within the jackets is unnecessary. Inline coupling jackets, described below in section 2b(2)(c1) and shown in FIGS. 36A thru 36E, and 37A thru 37D consist of two halves, each cylindrical, joined side by side by a common hinge, or spine, with spring at the junction of the halves at the jacket periphery.

Only the portion of the hinge extending over the inner of the half-jackets as shown in FIG. 37A, for example, is spring loaded to close when a trip is released as explained below in section 2b(2)(c1). The junction between the jacket halves is where the artery transecting razor and the mechanism that actuates it as shown in FIGS. 37C, 37E, and 37F is positioned. Optogenetics in the treatment of urinary bladder control and infection is addressed below in section 2c, entitled Ductus Side-entry Jackets and Vascular Valves in Urinary Diversion (optogenetic stimulation for control of a dysfunctional urinary bladder is also referenced below in this section.

Eventually, where the patient had already been admitted for some other reason, an automatic response system would already have been implanted as to require only incorporating the pertinent subroutine or subroutines and implanted sensors to detect procedure associated analytes such as rejection indicia. The totally implanted system then performs a number of procedure preparatory diagnostic steps. Implantable in relatively little time to commence a sudden compound vascular bypass, or sudden switched-organ, transplant and preprocedurally for a metered switch transplant, the automatic control system microprocessor administers the procedure in accordance with its prescription-program.

Here, where stressed is the correction of serious defects in the very young before irreversible degradation in cognitive and metabolic function results and compliance to the postoperative prescription regimen by the infant or baby-sitter is of key importance, the automatic system releases medication to include immunosuppressive in a timely manner, requiring only that the drug reservoirs implanted subcutaneously in the pectoral region as shown in FIGS. 26A thru 26C and 27C are timely replenished. The same applies to any other cognitively impaired patient having undergone a transplant.

### Compound Vascular Bypass Doubly Heterotopic Accessory Heart Transplantation

### Vascular Connection Schemes

Compound vascular bypass heterotopic double heart transplantation allows different schemes for connecting the added, or accessory, heart to the circulatory system in the recipient. Unlike previous schemes, of which a few are enumerated below in section 2b(1)(b)(b17), entitled Doubly Heterotopic Double Heart Compound Vascular Bypass, or Switched-organ Transplantation, intracardiac reconstructive surgery of the graft organ and sectioning of both the graft and host organs is eliminated, as are ischemia, cardiopulmonary bypass, cardioplegia, and except in young children and the disoriented, general anesthesia.

Moreover, despite the fact that positioning a second beating, vigorously working heart in the abdomen imposes a significant increased need for oxygen, previous schemes have not expressed concern for more than adding pumping power. However, based upon the ability of the respiratory system to provide a twenty percent increase in oxygen to defray a fifteen percent increase in metabolic rate, such an imposition of a significant load on the respiratory system is accommodated during normal gestation (see, for example, Kepley, J. M., Bates, K., and Mohiuddin, S. 2022. “Physiology, Maternal Changes,” Online, Treasure Island, Florida: StatPearls Publishing Co.; Newton, E. R. and May, L. 2017. “Adaptation of Maternal-Fetal Physiology to Exercise in Pregnancy: The Basis of Guidelines for Physical Activity in Pregnancy,” Online, Clinical Medicine Insights: Women's Health 2017:10; Soma-Pillay, P., Nelson-Piercy, C., Tolppanen. H., and Mebazaa, A. 2016. “Physiological Changes in Pregnancy,” Online, Cardiovascular Journal of Africa 27(2): 89-94).

However, the endocrinological adaptations reserved for pregnancy would not likely be triggered because a second heart had been connected to the abdominal aorta and the inferior vena cava, which would more likely stun the inherent ability of the respiratory system to adapt to an increased load which given an interval to adapt a supplementary direct path to a pulmonary vein might conceivably ameliorate. In the absence of a precedent, it can be speculated that to furnish the added heart with a direct path to a pulmonary vein, the increase in proximity to the site of gas exchange and greater summary luminal cross-section if it does not provide improved oxygenation from the outset, might prompt adaptation in the form of increased alveolar density, for example, supported by the directly pipe-targeted delivery of stem cells.

The druglines to each valve include the pulmonary veins and allow the directly pipe-targeted delivery of stem cells with the object of assisting alveolar proliferation. References pertaining to lung regeneration are cited below in this section. However, since without the addition of respiratory means such as addressed below in section 2b(1)(b)(b5), entitled Heterotopic Double Heart Metered Compound Vascular Bypass, or Switched-organ Transplantation [and so on], the load and the means for its defrayal might remain unaffected or respond too slowly, such means, however seductive, might have no effect. Short term burdening of the respiratory system where time did not allow for adaptation was seen in the cross-circulation that implemented the advent of open-heart surgery, where it allowed an adult to support a small child but not another adult.

If providing a direct path with increased summary luminal cross-section to a pulmonary vein makes it easier to respond and adapt to the added load more quickly and thoroughly than does simply increasing the burden without an attempt at offsetting measures, the option for direct connection afforded by compound vascular heterotopic heart transplantation will allow exploiting this factor. Application in the very young may exhibit better adaptation due to the plasticity of the respiratory system during development, to include its motor control and histology.

Long-term burdening which prompts adaptation naturally selected over time is seen in populations living at high altitudes (see, for example, Ortiz-Prado, E., Mendieta, G., Simbana-Rivera, K., Gomez-Barreno, L., Landazuri, S., and 3 others 2022. “Genotyped Indigenous Kiwcha Adults at High Altitude are Lighter and Shorter Than Their Low Altitude Counterparts,” Online, BioMed Central Journal of Physiological Anthropology 41(1): 8) and in certain cases of conjoined, parasitic, dipygus, and acardiac twins who shared the same respiratory system throughout early development. Precedential positioning of an accessory heart in the abdomen are no less dependent upon the ability of the body to provide the additional gas exchange but do so without providing means as would facilitate the process of adaptation. Plasticity of the respiratory system pertains to alveolar generative and regenerative potency and motor control.

Respiratory adaptation is multifactorial, involving the brainstem medulla and pons respiratory center as well as changes noted at the histological level (see, for example, Mallet, R. T., Burtscher, J., Pialoux, V., Pasha, Q., Ahmad, Y., Millet, G. P., and Burtscher, M. 2023. “Molecular Mechanisms of High-altitude Acclimatization,” Online, MDPI [Multidisciplinary Digital Publishing Institute] International Journal of Molecular Sciences (Basel, Switzerland) 24(2): 1698; Ortiz-Prado, E., Encalada, S., Mosquera, J., Simbana-Rivera, K., Gomez-Barreno, L., and 7 others 2022. “A Comparative Analysis of Lung Function and Spirometry Parameters in Genotype-controlled Natives Living at Low and High Altitude,” Online, BioMed Central Pulmonary Medicine 22(1): 100; Horscroft, J. A., Kotwica, A. O., Laner, V., West, J. A., Hennis, P. J., and 19 others 2017. “Metabolic Basis to Sherpa Altitude Adaptation,” Online, Proceedings of the National Academy of Sciences of the United States of America 114(24): 6382-6387; Hsia, C. C., Hyde, D. M., and Weibel, E. R. 2016. “Lung Structure and the Intrinsic Challenges of Gas Exchange,” Online, Comprehensive Physiology 6(2): 827-895; Kinkead, R., Bach, K. B., Johnson, S. M., Hodgeman, B. A., and Mitchell, G. S. 2001. “Plasticity in Respiratory Motor Control: Intermittent Hypoxia and Hypercapnia Activate Opposing Serotonergic and Noradrenergic Modulatory Systems,” Comparative Biochemistry and Physiology Part A: Molecular and Integrative Physiology 130(2): 207-218; Hsia, C. C., Dane, D. M., Estrera, A. S., Wagner, H. E., Wagner, P. D., and Johnson, R. L. 1985. “Shifting Sources of Functional Limitation following Extensive (70%) Lung Resection,” Online, Journal of Applied Physiology 104(4): 1069-1079; Mitchell, G. S. and Johnson, S. M. 1985. “Neuroplasticity in Respiratory Motor Control,” Online, Journal of Applied Physiology 94(1): 358-374; Takeda, S., Hsia, C. C., Wagner, E., Ramanathan, M., Estrera, A. S., and Weibel, E. R. 1985. “Compensatory Alveolar Growth Normalizes Gas-exchange Function in Immature Dogs after Pneumonectomy,” Online, Journal of Applied Physiology 86(4): 1301-1310).

Connection of an accessory heart transplanted by compound vascular bypass can be:
1. As shown in FIG. 16 where each corresponding vessel in either heart is connected and the accessory heart positioned in the chest to the right of that native or earlier transplanted.
2. As shown in FIG. 16 where the accessory heart is positioned outside the chest, usually in the abdomen without aortocaval connection or secondary connection to at least one pulmonary vein in the chest.
3. As shown in FIG. 16 where the accessory heart is positioned outside the chest, usually in the abdomen with direct aortocaval connection.
4. As shown in FIG. 16 where the accessory heart is positioned outside the chest, usually in the abdomen with direct aortocaval connection as well as secondary connection to at least one pulmonary vein in the chest.
5. As shown in FIG. 16 where the accessory heart is positioned outside the chest, usually in the abdomen with secondary connection to at least one pulmonary vein in the chest.
6. In the abdomen by direct aortocaval connection without direct connection to the native or earlier transplanted heart; that is, a precedential connection as cited below in section 2b(1)(b)(b16), entitled. Direct Aortocaval Connection: Unified Entry Arterial and Exit Venous Confluence Conduits for the Heterotopic Connection of a Graft Heart in the Lower Abdomen.
7. As in item 6 preceding where the accessory heart is directly connected aortocavally but then repositioned in the chest without direct connection to the native or earlier transplanted heart.
8. As in item 6 above where the accessory heart is directly connected aortocavally but then repositioned in the chest with direct connection to at least one pulmonary vein of the native or earlier transplanted heart.
9. By direct aortocaval connection wherein each oxygenated blood carrying vessel of the donor heart is connected to the abdominal aorta of the recipient and each deoxygenated blood carrying vessel of the donor heart is connected to the inferior vena cava of the recipient, the heart then elevated to the right of the native or earlier transplanted heart and connected vessel to corresponding vessel across the chest as depicted in FIG. 16. Connections to the donor heart prior to harvesting, and thereafter to the primary heart in the in the central compartment of the mediastinum are through unified entry arterial and venous confluence conduits.

Compound vascular bypass transplantation of a second, or accessory, heart and a doubly heterotopic heart positioned in the lower abdomen are addressed below in sections:
2b(1)(b)(b5), entitled Heterotopic Double Heart Metered Compound Vascular Bypass, or Switched-organ Transplantation and the Use of Unified Entry Arterial and Venous Confluence Conduits to Avoid Disuse Atrophy when the Graft Organ is Positioned in the Lower Abdomen by Direct Aortocaval Connection;
2b(1)(b)(b14), entitled Confluence Conduits: Connection of One or More Bloodlines to flow through a Common Channel to the Same or a Different Number of Bloodlines with Self-leveling Apportionment of Outflow;
2b(1)(b)(b16), entitled Direct Aortocaval Connection: Unified Entry Arterial and Exit Venous Confluence Conduits for the Heterotopic Connection of a Graft Heart in the Lower Abdomen; and
2b(1)(b)(b17), entitled Doubly Heterotopic Double Heart Compound Vascular Bypass, or Switched-organ Transplantation; and
2b(1)(b)(b18), entitled Hybrid Doubly Heterotopic Double Heart Transplantation.

Placement of an accessory heart in the lower abdomen is considered only when the condition of the patient will not allow placement in the chest. In such a case, a patient who should not have a lung resected, or one presenting with a severe surgical wound in the chest following resection of a tumor, for example, or due to an earlier disease, an accident, or war injury, the graft may have to remain in the lower abdomen. The added, or accessory, heart can be positioned thus in a heterotopic double heart transplant where connection to the native or earlier transplanted heart in the chest is vessel-to-corresponding vessel to include those of the pulmonary circulation. A heterotopic double heart transplant positioned in the abdomen can be ‘pure’ in omitting connection directly to the abdominal aorta and inferior vena cava locally, however, secondary connection thus is closer to the lungs, readily feasible, and will likely increase the level of oxygenation throughout the circulatory system.

In a direct aortocaval connection, the object is to augment pumping power. That positioning an accessory heart in the abdomen has been done several times is documented below in section 2b(1)(b)(b5), entitled Heterotopic Double Heart Metered Compound Vascular Bypass, or Switched-organ Transplantation [and so on]. Connection to the lungs to provide oxygenation to the entire body is indirect through the abdominal aorta, and therefore left to the pulmonary circulation of the native or earlier replaced, that is, the primary heart in the chest and the pulmonary circulation immediately associated with it. However, positioned thus, the highly active added myocardium itself consumes much oxygen, reducing the residual level of oxygen available to the rest of the infrarenal anatomy.

Nevertheless, as documented below in section 2b(1)(b)(b16), entitled Direct Aortocaval Connection: Unified Entry Arterial and Exit Venous Confluence Conduits for the Heterotopic Connection of a Graft Heart in the Lower Abdomen, as delineated in the literature, precedential connection thus does not involve a more direct connection to the lungs through the pulmonary vessels, much less elevating the accessory heart to the right of that native or earlier replaced heart in the chest and still less, additionally connecting the pulmonary vessels of the two hearts. Thus, following a direct aortocaval connection according to precedent, the graft heart remains in the lower abdomen with pulmonary communication through the abdominal aorta and inferior vena cava without cross-chest connection. Such a transplant is doubly heterotopic.

Accordingly, the issue of an additional secondary direct connection to the vessels of the pulmonary circulation across the chest does not arise. As presented here, however, provided space can be created to the right of the native or earlier replaced heart in the chest, a direct aortocaval connection can comprehend secondary connection thus, referred to as a direct aortocaval compound vascular heart transplant doubly heterotopic in secondarily positioning the accessory heart in the chest. Placement thus in itself does not necessarily involve cross-chest connections; however, here this is advocated and referred to as a hybrid doubly heterotopic heart transplant.

All connections between the vessels of the two hearts are like those shown in FIG. 15A. When the hearts are substantially alike in size and force of ejection, the diversion chutes will be set to the half open position with flow of the tributary streams confluent in direction. Whether a compound vascular heterotopic double heart transplant with the accessory heart positioned in the abdomen and secondarily connected to the abdominal aorta and the inferior vena cava, and an aortocavally connected transplant with the accessory heart placed in the chest and secondarily connected to the native or earlier transplanted heart across the chest will be completely convergent is unlikely: the former begins by connecting all of the cardiac vessels, and the latter secondarily places direct connection across the chest, and will not likely include all of the vessels as does the former to include the pulmonary veins.

The lack of a full skeletal protective surround requires the use of a heart cage as described below in section 2b(1)(b)(b6), entitled Heart Cage for the Infrarenal Abdominal Positioning of a Second Heart. A heart cage is shown in FIG. 24. By contrast, in both a doubly heterotopic double heart compound vascular bypass and a hybrid doubly heterotopic double heart transplant, the actual connections of the graft heart to the recipient are in the lower abdomen to the abdominal aorta and inferior vena cava by direct aortocaval connection. That is, through valves on the bloodlines at the lower end of the elongated arterial and venous conduits which allow the graft itself to be positioned in the chest to the right of the native or earlier transplanted heart.

In the abdomen, connection is specifically of the oxygenated blood-carrying vessels of the graft to the abdominal aorta through the valves at the ends of the bloodlines at the lower end of the elongated arterial confluence conduit, while connection of the deoxygenated blood-carrying vessels of the graft is to inferior vena cava of the recipient through the valves at the ends of the bloodlines at the lower end of the elongated venous confluence conduit. The hybrid doubly heterotopic double heart transplant is connected to the recipient in the lower abdomen as in a doubly heterotopic double heart transplant with the graft actually positioned to the right of the native or earlier transplanted heart through elongated arterial and venous confluence conduits.

However, the hybrid doubly heterotopic double transplant differs in that rather than to depend solely upon the connections in the lower abdomen as in the doubly heterotopic transplant, once positioned in the chest, local arterial and venous confluence conduits are used to connect the oxygenated blood carrying vessels of the graft directly to those of the native or earlier transplanted heart and the deoxygenated blood carrying vessels of the graft directly to the deoxygenated blood carrying vessels of the native or earlier transplanted heart. This arrangement is intended to take advantage of the closer proximity to the lungs for oxygenation. Briefly stated, a hybrid is the same as a doubly heterotopic with the vessels additionally connected across the chest.

More specifically, the aortae and pulmonary veins carrying oxygenated blood are usually connected through an arterial confluence conduit and the superior venae cavae and pulmonary trunks, or right and left pulmonary arteries, carrying deoxygenated blood, are usually connected through a venous confluence conduit. With this method, the graft in a hybrid doubly heterotopic heart transplant has two valves per vessel-one leading down to its direct aortocaval connection and the other linking each vessel to its counterpart in the native or earlier transplanted heart.

This and the absolute number of vascular valves or servovalves required in a compound bypass transplant illustrate the importance of light weight and small size as well as an inordinate degree of dependability in vascular valves and servovalves. In a sudden switch procedure, all such connections are made through plunger solenoid-driven valves, while in a metered switch procedure, the valves are driven by servomotors such as of the pulse tubular linear type controlled by negative feedback to advance to the succession or stream of set points directed by the implanted prosthetic disorder response system prescription-program.

Whether 1. Heterotopic, using ordinary heart-to-heart artery-to-artery and vein-to-vein connections as in an orthotopic heart switched organ transplant with the valves, as shown in FIG. 15A, set—depending upon the relative sizes of the hearts—at about halfway open to serve both hearts, 2. Doubly heterotopic through indirect aortocaval connection with the second heart then positioned in the chest, or 3. Hybrid in being connected as in a doubly heterotopic transplant but with the aortae and pulmonary vessels exceptionally connected directly to the innate or earlier transplanted heart with the valves, as shown in FIG. 15A, set—depending upon the relative sizes of the hearts—at about halfway open to serve both hearts as pertains to option 1, meaning a switched-organ heterotopic double heart transplant where all the vessels are connected thus.

Valving of the confluence conduit bloodlines in the abdomen when the heart is to be positioned there consist of a one large valve and bloodline each for connection to the aorta and the inferior vena cava at the end proximal thereto, and valves and bloodlines for each vessel of the graft at the end connected to the graft. As to the methods used to position an aortocavally connected graft in the chest, the hybrid doubly heterotopic transplant is the same as a doubly heterotopic transplant which then has cross-chest valves and bloodlines added for local connection to the primary heart.

Accordingly, the elongated confluence conduits to span the distance from the lower abdomen to the chest are the same in both the doubly heterotopic and the hybrid methods. The elongated arterial and venous confluence conduits used when connection is aortocaval but the heart to be positioned in the chest have one each large caliber bloodlines equipped with valves for connection to the abdominal aorta and inferior vena cava. That arterial has bloodlines equipped with valves for connection to the oxygenated conducting vessels of the graft in the chest while that venous has the same for connection to the deoxygenated vessels.

In the chest, however, the hybrid method calls for a second valve per vessel to connect not only to the proximal end of the arterial and venous confluence conduits but also to the graft aorta and pulmonary vessels. Connections to vessels conveying oxygenated and unoxygenated blood can be segregated into separate shorter confluence conduits. Those oxygenated include the aorta and the pulmonary veins, while those deoxygenated include the pulmonary artery and the superior vena cava. As do all bloodlines, the elongated arterial and venous confluence conduits used to span the distance from the lower abdomen require a nonkinkable section at the waist.

Bloodlines and confluence conduits for use both in children have a clot-repelling internal surface, usually fluoropolymeric with additional coating as necessary. This notwithstanding, the ability of the implanted prosthetic disorder response system to administer a slow heparin or other antithrombotic drip into the druglines of the valves and/or confluence conduits provides additional protection against obstruction of a bloodline by clot. Except that its materials must be biocompatible, the reinforced segment traversing the waist can incorporate circumferential furrows to flex without resistance.

Doubly heterotopic positioning of a second heart in the lower abdomen by direct aortocaval connection consists of connecting each donor heart oxygenated blood outlet vessel to the native aorta and each unoxygenated inlet vessel to the native inferior vena cava. For vessels similar in caliber, confluence conduits can be used. Except for the hybrid doubly heterotopic connection, despite having no local connection to the lungs, the normal return pressure of blood against gravity through the inferior vena cava indicates that the need for inline booster pumps in the confluence conduits to propel unoxygenated blood from the abdomen to the chest is considered unlikely.

If the lungs are impaired, consideration should be given to performing a heart-lung transplant. Otherwise, with two competent lungs, an insufficiency of oxygenation should it arise is dealt with below in section 2b(1)(b)(b16), entitled Direct Aortocaval Connection: Unified Entry Arterial and Exit Venous Confluence Conduits for the Heterotopic Connection of a Graft Heart in the Lower Abdomen.

In doubly heterotopic double heart transplantation, the vessel connections are aortocaval as in the heterotopic positioning of a second heart in the lower abdomen but the heart is positioned adjacent to that native or earlier transplanted in the chest, requiring elongated confluence conduits with a reinformed section to pass the waist. A hybrid doubly heterotopic double heart transplant is similar to a doubly heterotopic double heart transplant in that it is placed in the chest to the right of the native or previously implanted heart, space to place the accessory second heart, which, if necessary, can be made smaller by removing the lower portion of the right lung.

The lung will then regenerate within the smaller space by increasing the density of alveoli (see, for example, Ali, M., LaCanna, R., Lian, Z., Huang, J., Tan, Y., and 3 others 2022. “Transcriptional Responses to Injury of Regenerative Lung Alveolar Epithelium,” Online, iScience 25(8): 104843; Ali, M., Zhang, X., LaCanna, R., Tomar, D., Elrod, J. W., and Tian, Y. 2022. “MICUI [mitochondrial Ca2+ uptake 1]-dependent Mitochondrial Calcium Uptake Regulates Lung Alveolar Type 2 Cell Plasticity and Lung Regeneration,” JCI [journal of clinical investigation] Insight 7(4): e154447; Hynds, R. E. 2022. “Exploiting the Potential of Lung Stem Cells to Develop Pro-regenerative Therapies,” Online, Biology Open 11(10): bio059423; Liberti, D. C., Liberti, W. A. 3d., Kremp, M. M., Penkala, I. J., Cardenas-Diaz, F. L., an 5 others 2022. “KIfs [transcription factor] Defines Alveolar Epithelial Type 1 Cell Lineage Commitment during Lung Development and Regeneration,” Developmental Cell 57(14): 1742-1757; Alysandratos, K. D., Herriges, M. J., and Kotton, D. N. 2021. “Epithelial Stem and Progenitor Cells in Lung Repair and Regeneration,” Online, Annual Review of Physiology 83:529-550; Khedoe, P. P. P. S., Wu, X., Gosens, R., and Hiemstra, P. S. 2021. “Repairing Damaged Lungs Using Regenerative Therapy,” Online, Current Opinion in Pharmacology 59:85-94; Liberti, D. C., Kremp, M. M., Liberti, W. A. 3d., Penkala, I. J., Li, S., Zhou, S., and Morrisey, E. E. 2021. “Alveolar Epithelial Cell Fate is Maintained in a Spatially Restricted Manner to Promote Lung Regeneration after Acute Injury,” Online, Cell Reports 35(6): 109092; Basil, M. C., Katzen, J., Engler, A. E., Guo, M., Herriges, M. J., and 11 others 2020. “The Cellular and Physiological Basis for Lung Repair and Regeneration: Past, Present, and Future,” Online, Cell Stem Cell 26(4): 482-502; Basil, M. C. and Morrisey, E. E. 2020. “Lung Regeneration: A Tale of Mice and Men,” Online, Seminars in Cell and Developmental Biology 100:88-100; Lai, Y.-T., Chao, H.-W., Lai, A. C.-Y, Lin, S.-H., Chang, Y.-J., and Huang, Y.-S. 2020. “CPEB2 [cytoplasmic polyadenylation element-binding protein 2]-activated PDGFRalpha [platelet-derived growth factor receptor alpha] mRNA Translation Contributes to Myofibroblast Proliferation and Pulmonary Alveologenesis,” Online, BioMed Central Journal of Biomedical Science 27(1): 52; Lucas, A. Yasa, J., and Lucas, M. 2020. “Regeneration and Repair in the Healing Lung,” Online, Clinical and Translational Immunology 9(7): e1152; Parekh, K. R., Nawroth, J., Pai, A., Busch, S. M., Senger, C. N., and Ryan, A. L. 2020. “Stem Cells and Lung Regeneration,” Online, American Journal of Physiology. Cell Physiology 319(4): C675-C693; Raslan, A. A. and Yoon, J. K. 2020. “WNT ([Wingless and Int-1] transduction pathways that gain entry into the cell through its surface receptors) Signaling in Lung Repair and Regeneration,” Online, Molecules and Cells 43(9): 774-783; Wu, A. and Song, H. 2020. “Regulation of Alveolar Type 2 Stem/Progenitor Cells in Lung Injury and Regeneration,” (in English) Online, Acta Biochimica et Biophysica Sinica 52(7): 716-722; Kotton, D. N. and Morrisey, E. E. 2014. “Lung Regeneration: Mechanisms, Application, and Emerging Stem Cell Populations,” Online, Nature Medicine 20(8): 822-832; references up to 2019 cited below in section 2b(1)(b)(b5), entitled Heterotopic Double Heart Metered Compound Vascular Bypass, or Switched-organ Transplantation [and so on].

In a hybrid doubly heterotopic double heart transplant, connection of the graft organ is not directly aortocaval. Rather the heart is positioned in the chest with its primary connections between the graft aorta and a recipient abdominal aorta and graft vena cavae to the recipient inferior vena cava through elongated and waist segment reinforced confluence conduits, the aorta and pulmonary arteries and veins exceptionally connected directly vessel-to-vessel between the adjacent hearts across the chest with the valves half open to serve both hearts as in a double heart transplant, the object being to improve the efficiency of oxygenation. Oxygenation and means for proving supplementary oxygenation are dealt with below in section 2b(1)(b)(b16), entitled Direct Aortocaval Connection: Unified Entry Arterial and Exit Venous Confluence Conduits for the Heterotopic Connection of a Graft Heart in the Lower Abdomen.

Switched-organ orthotopic replacement of an unsurvivable heart in a prenate or infant or in an adult with end stage heart failure is always to be preferred. Heterotopic procedures are especially suitable for a neonate or infant with an urgent need for a normal heart where none is available or for an adult with end stage heart failure where the need for a replacement heart cannot be met, and in parts of the world where a more orthodox remedy such as a ventricular assist device is unaffordable. In places where infant death is common, a child can usually be provided with a competent heart through orthotopic switched-organ transplantation.

Relatively quick, less complicated, and having eliminated the trauma of surgery other than that imposed by the need for an entry wound, switched-organ transplantation should allow optimized regeneration of the severed vagus nerve, a gradual acquisition of chronotropic competency, and therewith, more nearly normal function. In these places, the procedure may have to be administered by physicians without the aid of a prosthetic disorder response system. When possible, the need for such an automatic system to signal and respond to any follow-up exigencies and dispense medication in accordance with the prescription should be obtained by referral to a country having this capability.

For a patient born with both a congenitally malformed heart and aorta or other great vessel or vessels, the method of heart transplantation described herein can be combined with the method for replacement of the defective vessel or vessels. Consequences to take into account include denervation of the replacement heart as well as the loss and the prospects for reconstitution of great vessel baroreception and chemoreception. When the heart is to be transplanted as well, the replacement of one or more severely malformed great vessels is best by including these in the allograft.

If this is impracticable or a suitable graft unavailable, the replacement of a severely malformed great vessel is with a prosthesis. In this case, because they support the substrate vessel, the loss of vasa vasorum and vasa nervorum is of little if any consequence. To reduce operative time and stress, these are best offset in time, which also allows the procedural administrative programs executed by the automatic disorder response system to concentrate on either procedure without the programming and executive complexity of performing both simultaneously.

Compared to saving the patient, denervation sequelary to harvesting, usually recovered over time anyway, is of little consequence (see, for example, American Association for the Advancement of Science EurekAlert! 2002. “Post-transplant Nerve Regrowth Better with Young Heart, Quick Surgery,” online at eurekalert.org/news-release)(see also Christensen, A. H., Wyller, V. B. B., Nygaard, S., Rolid, K., Nytroen, K., and 5 others 2023. “Factors Associated with Sinoatrial Reinnervation after Heart Transplantation,” Online, Transplantation Direct (Philadelphia, Pennsylvania) 9(12): e1553; Salamon, R. J., Halbe, P., Kasberg, W., Bae, J., Audhya, A., and Mahmoud, A. I. 2023. “Defining Cardiac Nerve Architecture during Development, Disease, and Regeneration,” Online, bioRxiv [bio archive] the Preprint Server for Biology, [in Press], January 3:2022.12.31.522405; Salamon, R. J., Halbe, P., Kasberg, W., Bae, J., Audhya, A., and Mahmoud, A. I. 2023. “Parasympathetic and Sympathetic Axons are Bundled in the Cardiac Ventricles and Undergo Physiological Reinnervation during Heart Regeneration,” Online, [Internet] Science (Cambridge, Massachusetts) 26(10): 107709; Squires, R. W. 2023. “Cardiac Transplant and Exercise Cardiac Rehabilitation,” Heart Failure Reviews (Norwell, Massachusetts) 28(6): 1267-1275; Dipchand, A. I. and Laks, J. A. 2020. “Pediatric Heart Transplantation: Long-term Outcomes,” Online, Indian Journal of Thoracic and Cardiovascular Surgery 36 (Supplement 2): 175-189; Clemmensen, T. S., Eiskjaer, H., Logstrup, B. B., Valen, K. P. B., Mellemkjaer, S., and Poulsen, S. H. 2019. “Prognostic Value of Exercise Myocardial Deformation Haemodynamics in Long-term Heart-transplanted Patients,” Online, ESC [European Society of Cardiology] Heart Failure (Oxford, England) 6(4): 629-639; Grupper, A., Gewirtz, H., and Kushwaha, S. 2018. “Reinnervation Post- heart Transplantation,” Online, European Heart Journal 39(20): 1799-1806; Awad, M., Czer, L. S., Hou, M., Golshani, S. S., Goltche, M., and 8 others 2016. “Early Denervation and Later Reinnervation of the Heart following Cardiac Transplantation: A Review,” Online, Journal of the American Heart Association (Oxford, England) 5(11): e004070; Lundgren, J. and Radegran, G. 2015. “Hemodynamic Characteristics Including Pulmonary Hypertension at Rest and during Exercise before and after Heart Transplantation,” Online, Journal of the American Heart Association (Oxford, England); 4(7): e001787; Nytroen, K. and Gullestad, L. 2013. “Exercise after Heart Transplantation: An Overview,” Online, World Journal of Transplantation (Pleasanton, California) 34): 78-80; various articles to include Bengel, F. M., Ueberfuhr, P., Schiepel, N., Nelolla, S. G., Rechart, B., and Schwaiger, M. 2001. “Effect of Sympathetic Reinnervation on Cardiac Performance after Heart Transplantation,” Online, New England Journal of Medicine 345(10): 731-738; Pope, S. E., Stinson, E. B., Daughters, G. T. 2nd, Schroeder, J. S., Ingels, N. B. Jr., and Alderman, E. L. 1980. “Exercise Response of the Denervated Heart in Long-term Cardiac Transplant Recipients,” American Journal of Cardiology (New York, New York) 46(2): 213-218; Norvell, J. E. and Lower, R. R. 1973. “Degeneration and Regeneration of the Nerves of the Heart after Transplantation,” Transplantation 15(3): 337-344).

As addressed below in section 2b(2)(f), entitled Expandable Structure to Accommodate Growth and Self-adjust in Size in Vascular Segment Prostheses, Lines, and Confluence Conduits Placed in Children, for use in a child, expandable replacement vascular segments allow for growth without the need for painful open chest revisions. The concept having originated earlier, the first published attempt at the transplantation of a second, or accessory, heart by Carrel and Guthrie appeared in 1905, (see, for example, Cooper, D. K. C. 1968. “Experimental Development of Cardiac Transplantation,” Online, British Medical Journal 4(5624): 174-181).

These early attempts sought answers not to the practical feasibility of placing a second heart in a human as a life-saving measure, which was entirely beyond the medical acumen and means available at the time, but rather answers to basic physiological questions, such as whether if left to its integral beat-discharging nodes alone, an excised heart denervated of its vagus sympathetic and parasympathetic nerve fibers would continue to provide adequate circulatory function.

As indicated, these experiments were understood from the outset to be unavailing in terms of direct applicability to the practical transplantation of an additional heart or the replacement of that native in a human, indeed, the procedures were performed without regard to aseptic technique, by then well established as imperative to avoid infection, making it clear that no such practical application was expected for the near future. Unlike the all-inclusive connections between corresponding major vessels of the donor and recipient organs prescribed by the compound vascular bypass technique of transplantation, in these early experiments, donor-recipient organ arteriovenous anastomoses were few as to justify description as little more than tokenal and clearly inadequate.

It is this all-inclusive connection that eludes the atrophy which inevitably affected the less than fully connected accessory heart, and it is the avoidance of anoxia at the outset that almost certainly forestalls if not overcomes the probable cardiac allograft vasculopathy that cause the graft to fail even after a decade. The graft organ was essentially rendered parasitic on that native, left with an insufficiency of perfusion that would have caused it to fail in any event, so that viewed in retrospect, the lack of immunosuppressives, an additionally unmet critical necessity, can be considered superfluous.

Preceding the medical application of immunosuppressives—notably cyclosporine in 1983—experimental pathologists and cardiopulmonologists used the period to gain background information such as electrocardiogramanceming heart and heart and lung transplantation performed in laboratory animals using different techniques of transplantation available at the time (see, for example, Lee, S., Willoughby, W. F., Smallwood, C. J., Dawson, A., and Orloff, M. J. 1970. “Heterotopic Heart and Lung Transplantation in the Rat,” American Journal of Pathology 59(2): 279-298). However, as specified below in section 2b(1)(b)(b2), entitled Prenatal Compound Vascular Bypass, or Sudden Switched-organ Transplantation of the Heart, cyclosporine and other immunosuppressives can produce serious side effects, and any means for reducing the need for these drugs—in particular the donor-recipient reciprocal microchimerization of extended reciprocal cross-circulation during metered solid organ transplantation—is beneficial.

Today, most of the complications encountered would no longer remain as obstructions to organ transplantation even in conventional treatment where drugs are dispersed throughout the circulation, much less in compound vascular bypass-type solid organ transplantation where the adverse side effects of drugs can be substantially eliminated through the direct pipeline-targeting of drugs to trouble spots instantly upon detection of the diagnostic symptoms by prepositioned implanted sensors. Diagnosis and therapy applied to either of the separated hearts in a double heart transplant can be substantially isolated, and this means that the performance of each heart in contributing to the combined effort can be separately adjusted and this adjustment applied continuously by the master controller.

FIG. 15A provides a summary representation of the vascular connections in a compound vascular bypass, or sudden switched-organ orthotopic heart transplant. That the diversion chutes are centered in the lumina indicates that the process of transfer from the circulatory system of the donor into that of the recipient is gradual, or metered, rather than sudden as it would be in an emergency, meaning that the process is negative feedback responsive, and that it has progressed to about the halfway point—about, because the sizes of the hearts and the absolute stroke volume, or ejection fraction, of each is a determinant in just where the halfway point lies.

If at this point in what would more often continue as a compound vascular bypass orthotopic heart transplant, rather than to continue the transfer so that the donor heart replaces that of the recipient, the transfer of blood flow, that is, the continued progress of the diversion chutes, is suspended and the donor heart implanted in the recipient as a second, backup or accessory heart, one has a compound vascular bypass type double, or heterotopic, heart transplant. In that each heart remains intact, a compound vascular bypass transplant is fundamentally different than either a conventional heterotopic heart transplant as developed under the aegis of Barnard or any of the many forms of attachment of the accessory heart to the circulatory system of the recipient described in experiments over the years.

In the latter, each heart does remain intact but with unsurvivably inadequate perfusion and with rare exceptions, placement such as in the neck, which cannot even be considered for application in a heart patient. The intactness and separation between the two hearts alone makes a compound vascular bypass, or sudden switched-organ heterotopic heart transplant fundamentally superior to a conventional heterotopic heart transplant. Where this distinctness equates to diagnostic and therapeutic flexibility, the hearts in a conventional heterotopic transplant are merged.

Merged through extensive incision that inflicts considerable trauma on both hearts and renders each no longer separately addressable for diagnosis and treatment. The merger essential to allow the donor and recipient hearts to function as a unit, the secondary disengagement of the two is precluded. Any affectation of the one is the affectation of both. There is no option to remove the diseased component and preserve that unaffected. This preemptive elimination of ‘bailout’ options can prove critical.

Following a conventional heterotopic heart transplant, a subsequent failure requires removal of the post merger unitary heart. By contrast, following a compound vascular bypass transplant, either heart can be removed leaving the other in place to be treated as necessary. Following a compound bypass heterotopic double heart transplant, whether the graft is retained in the chest or resituated to the lower abdomen, the removal and replacement of either the native or earlier transplanted heart or the graft is addressed below in this section. Should the support of the donor heart have allowed the diseased heart to recover, or for that matter, the support of the inmate heart have allowed that accessory to recover, the other heart can be removed and made available for another well matched patient.

When both hearts are impaired so that their combined stroke volumes achieve normal output, the two would best beat coincidentally or with sufficient overlap to approximate substantially normal output to provide adequate pulmonary and systemic perfusion. Where the added heart is unimpaired and the two synchronized with the aid of an artificial pacemaker, the excessive output could result in injury to the finer vasculature and alveoli of the lungs. For this reason, a third advantage in a switch-type double heart transplant is an actual preference for using an accessory heart itself less than perfect in performance and trusting to the implanted automatic disorder response system to monitor and align the separate as well as the joint performance of the hearts through the release as necessary of drugs and electrostimulatory synchronization.

An inherent consequence of this perhaps counterintuitive eventuality is that unimpaired hearts and other organs often unavailable, transplantation of a second heart as accessory greatly increases the available pool of transplant organs. With brain death imminent, the prospective organ donor is maintained past death in a living condition with circulation and respiration sustained with the aid of electrical stimulation, hormones, enzymes, and drugs. If the heart and/or lungs must be harvested first, then the donor is supported past death with the aid of an extracorporeal membrane oxygenator.

Relatively short-lived in its effectiveness, conventional cardiopulmonary bypass is not used. When necessary, a cardioverter/pacemaker-defibrillator is used to assist in maintaining normal circulation. Breathing is best spontaneous, assisted by a ventilator, continuous a positive airway pressure mask, intubation, hood or nasal canula, or surgical chest dome, as described below in section 2b(2)(k) of like title, and shown in FIGS. 23A thru 23E. Variations as to the means of preservation, such as the use of a surgical chest dome are addressed at various points herein.

One object of the invention to bring solid organ transplantation, larger vessel segment replacement procedures, extracardiac correction of transposition of the great vessels, and other operations that ordinarily involve considerable trauma and require circulatory arrest through the affected segment, organ, or volume of tissue within the capability of general surgeons outside the largest centers. The need for a ventilator, which also requires inordinate expertise not found in smaller clinics, is another factor that detracts from reducing the degree of complexity. For example, avoiding the need for a ventilator and trained operator in lieu of the direct application of electrostimulation to the breathing centers in the brainstem calls for expertise; however, acquisition of the apparatus on an emergency basis, access to a neurologist, and the reduction of the procedure to a standard routine could prove not only more practicable but safer.

In this way the donor organ, even the heart and lungs, are maintained in a state of preservation least disagreeable to the immune system of the recipient as can best be attained. To minimize the potential injury, the pacemaker, or a ventilator if not precisely set (see, for example, Hall, Jesse 2006. “Respiratory Failure and Mechanical Ventilation,” Chapter 65, pages 544-555, in The Merck Manual, Whitehouse Station, New Jersey: Merck Research Laboratories; Kantak, A. D. 2006. “Respiratory Disorders in Neonates, Infants, and Young Children,” Chapter 277, page 2296, in The Merck Manual, Whitehouse Station, New Jersey: Merck Research Laboratories) or an electrostimulator can cause if overused and thus reduce the period that the deceased donor can be preserved, not only should the use of these be sparing but measures taken to minimize injury from these sources.

The donor organ is directly connected to the recipient organ for reciprocal cross-circulation and transfer before harvesting, so that the graft organ is never left without normothermal pulsatile perfusion much less face anoxia, and is spared the shocks of death and excision as well as preservation in a static or flowing chilled perfusate. Instead, eliminating the trauma of an abrupt cessation in corporeal integrity followed by severe wounding, the graft organ is transferred from the circulatory system of the donor into that of the recipient in one continuous operation without injury or interruption in perfusion.

As a result, unimpaired graft organs are significantly less, probably critically less, traumatized, and organs that would ordinarily be considered less than ideal for transplantation are made available, especially in an accessory role as in a double heart transplant. Such a considerable expansion in the pool of available organs increases the odds that a donor may be in the same center or one nearby, sparing the graft organ or organs an interval of disembodied isolation as well as the need to transport the donor to the recipient or the reverse. For this reason, the graft organ experiences little if any of the impact of having undergone the death of its host and the sudden loss of its anatomical and physiological context.

Essentially, the graft organ is spared the shock of death, the lesser shock of insertion into a milieu less than perfectly matched immunologically abated through the administration of immunosuppressives and anti-inflammatoires as necessary. In this way, the transition is made as atraumatic as possible. A much more readily available second heart differs from a ventricular assist device not only in sustaining pulsatile flow but in avoiding a cost for the equipment that is unaffordable throughout most of the world. The considerable trauma inflicted on both hearts in a conventional heterotopic heart transplant reduces the odds for success from the outset.

Both the native heart, already impaired, and the graft organ, chosen on the basis of strength and normal function, essential just to survive the surgical trauma to ensue, are not only traumatized but merged in a way that substantially precludes targeting treatment to either without affecting the other or disengaging the two were either to fail. Merged, the ascription to either heart as etiological source of a later complication will have been diagnostically obscured, with treatment applied no less to the wrong as to the right heart. Because of the injury involved, secondarily to separate the heterotopically joined hearts in order to permit either to be separately diagnosed or treated is not an option.

Necessitating risk and wounding equal to if not greater than that undergone to have joined the two, once accomplished, the patient with a failing heterotopic graft is committed to an orthotopic transplantation, if necessary, following the short term use of an artificial heart. By contrast, a compound vascular bypass, or sudden switched-organ double heart transplant involves no direct contact between the native and added hearts. For this reason, it makes possible the differential diagnosis and direct targeting of remedial medication or any other form of therapy or means of synchronization to either heart without necessarily involving and affecting the other.

Not preferred but rather reserved for conditions which preclude placement in the chest, an accessory heart placed using a compound vascular bypass technique can be located in an inferior position where its output must work against gravity, an inline pump then applied as necessary. Where revision of a conventional orthotopic, and especially a heterotopic heart transplant, is keenly traumatic in requiring the undoing of the extensive surgery that had been needed to unitize the donor and recipient hearts, the completely intact condition of both hearts following a heterotopic double heart transplant using the metered switch method not only expedites healing following the original and later operations but allows either heart to be retrieved and replaced with minimal trauma using the same switch method that had been used to perform the original operation.

In some cases, a heterotopic double heart transplant may allow the native heart to regain adequate function so that the added heart, not having been traumatized, can be reused. In third world countries where ventricular assist devices for use as bridges pending the availability of a good heart are unaffordable, a double heart transplant can not only extend the life of patients approaching or having reached, if not earlier, class III of the New York Heart Association staging scheme. However, unlike an assist device, providing pulsatile flow and normal oxygenation, a double heart transplant can remain in place indefinitely.

Such extends to adults an insistence upon prompt intervention to not just palliate or ameliorate but whenever possible, positively stop any process of progressive structural failure, and the same approach should apply throughout medical practice, from the immediate repair of the collapsing trachea in a small dog to the surgical repair or removal of an asymptomatic venous aneurysm in the lower leg before it is likely to prompt the formation of a thrombus that could eventuate as a pulmonary embolism (see, for example, Patel, R., Woo, K., Wakefield, T. W., Beaulieu, R. J., Khashram M., and 17 others 2022. “Contemporary Management and Outcomes of Peripheral Venous Aneurysms: A Multi-institutional Study,” Journal of Vascular Surgery. Venous and Lymphatic Disorders (New York, New York) 10(6): 1352-1358; Patel, R., Hanish, S., Baril, D., Woo, K., and Lawrence, P. 2019. “Contemporary Management of Lower Extremity Venous Aneurysms,” Journal of Vascular Surgery. Venous and Lymphatic Disorders (New York, New York) 7(6): 860-864). Such a repair is facilitated with the aid of the device described below in sections 2b(2)(c)(c2), entitled Automatic Insertion into a Ductus of an Inline Coupling Jacket-secured Bridging, or Tie-line and Its Replacement if Necessary, and shown in FIGS. 37A thru 37E, and 2b(2)(d), entitled Anoxia-free Aortic Repair: Aortic Prostheses Thoracic, Thoracoabdominal, and Abdominal.

Flow diversion implants can be classified into six types in three categories:

Category a. Flow diverters:
Type a1. Fixed diverters inserted into the free end of a transected ductus, as shown in FIGS. 31A and 31B.
Type a2. Bridge or tie-line-mounted fixed diverters positioned along a conduit to replace a segment of the ductus by means of the automatic sudden insertion using inline coupling jackets by means of the automatic sudden insertion using inline coupling jackets as described below in section 2b(2)(c)(c2), entitled Automatic Insertion into a Ductus of an Inline Coupling Jacket-secured Bridging, or Tie-line and Its Replacement if Necessary, and illustrated in FIGS. 37C, 37D, and 37C.

Category b. Either/or outlet switches:
Type b1. Side-entry bistable, or either or, highly damped nonsparking solenoid-driven valves directly connected to the ductus;
Type b2. Bridge or tie-line-mounted highly damped nonsparking solenoid-driven valves positioned along a conduit to replace a segment of the ductus by means of automatic sudden insertion using inline coupling jackets by means of automatic sudden insertion using inline coupling jackets.

Category c. Flow either/or switched-organs or apportioning splitters:
Type c1. Side-entry continuously variable servovalves directly connected to the ductus.
Type c2. One time upon placement side-entry continuously variable servovalves positioned along a conduit to replace a segment of the ductus by means of automatic sudden insertion using inline coupling jackets by means of iautomatic sudden insertion using inline coupling jackets.

By ‘fixed’ is meant that the diverter is incapable of switching flow to either of two effluent lines as can a solenoid-driven valve, much less apportioning flow between the two lines as can a servovalve. This rising order of capability—diversion of flow without switching ability, switchability of flow diverted to either of two outlet passageways, and continuously variable proportional switchability into either either of two outlet passageways—is associated with increasing complexity and cost. In any given situation, the most suitable device is that simplest, lightest in weight, and smallest which can perform the function required without causing discomfort or greater expense to buy and maintain.

Only side-entry devices, whether connected directly to the substrate ductus or along an insert bridge, or tie-line, are capable of switching flow when solenoid-driven as per category b, much less apportioning the flow between either of the two outlet ductus when servomotor-driven as per category c. The first two, end-inserted fixed diverters and conduit-mounted fixed diverters are not ductus side-entry devices and are not capable of switching flow between two outlet ductus as can a solenoid-driven valve, nor both switch thus and apportion flow through each outlet line as can a servo-driven valve. However,

1. The overall size and weight of the valves are always minimized.

2. The side stem protrudes in the direction of the shunt into which the outlet line is directed and flow is to be switched in any event.

3. The angle of the side stem relative to the barrel can be adjusted to other than ninety degrees.

4. The level and

5. The direction to which the side stem is set along the substrate ductus can be adjusted to achieve minimal if any protrusion into neighboring tissue.

6. The switches are enclosed within a cushioning wrap so that little if any irritation to neighboring tissue should result from any contact.

Taken together, these factors should provide sufficient flexibility in positioning to allow placement of either type switch in the chest for a switch heart transplant, for example. Thoracic and thoracoabdominal prostheses do not require switchability. A sudden compound vascular bypass, or sudden switched-organ, transplant as addressed below in section 2b(1)(a) entitled Sudden Compound Vascular Bypass, or Sudden Switched-organ, Transplantation requires switchability between its substrate ductus and prosthetic shunt, hence, solenoid-driven valves connected by direct or tie-line inserted side entry as shown in FIG. 36G.

Sudden compound vascular bypass, or sudden switched-organ, transplantation is reserved for exigent circumstances, the gradual blending of donor and recipient blood is always preferred when time allows. A metered switch transplant as addressed below in section 2b(1)(a) requires servomotor-driven valves. Whereas solenoid-driven valves cannot provide gradual or partial adjustment action, servomotor-driven valves more costly, can also provide quick action. Accordingly, where speed is important but not urgent, the option to convert from a sudden to a metered switch procedure midprocedurally if such is estimated to be much preferred is made possible by servovalves.

For use in the arterial tree, a central requirement in servovalve motors is that the holding torque rating or placement in a closed loop system be adequate to hold fast and not be overcome by the pulse which would otherwise displace the valve from its instantaneous commanded setting. In addition to expediting the placement of side-entry devices ordinarily connected directly to the substrate ductus, non-side-entry devices, such as independent drug delivery lines can be placed as having been incorporated along an insert bridge, or tie-line.

Examples include the direct sensor-driven delivery into the portal vein of insulin and the scheduled delivery of psychotropics such as neuroleptics and/or anxiolytics directly into either or both internal carotids. The ability to switch flow into either of two lumina comes with the sideways protrusion of the sidestem which inline coupling jackets, which lack this function, avoid. However, a side-entry jacket or valve can be encased within a cushioning outer layer, positioned at different levels along a line, and rotated however necessary to the angle that best accommodates the sidestem without encroachment upon neighboring tissue, and if necessary, the sidestem can be cushioned.

For use in an infant or small child where adaptation in size to accommodate growth is sought, valves cannot be provided in an expandable form as expandability connotes compressibility which would were it to affect a valve would interfere with the sliding function of the diversion chute. However, given the degrees of freedom in level and angle, the largest valves that can be accommodated should be used, enclosed within a cushioned outer jacket if necessary.

The lines used and the ends connected to the valves can be expandable. Other components of a fully implanted ambulatory prosthetic disorder response system are miniaturized and kept to a minimum. In an infant with multiple comorbidities, components which cannot be implanted are relegated to a padded flat pack to be worn in the small of the back. Much as a pacemaker-cardioverter-defibrillator, a prosthetic disorder response system is never intended for use in a patient for whom it is not absolutely necessary.

Inseparable from at least some measure of sideways protrusion associated with side-entry, the switching requirement in sudden compound vascular bypass, or sudden switched-organ, transplantation and the apportioning requirement in metered switch organ transplantation are the same whether each bloodline is separately run between each counterpart vessel between donor and recipient of a subset thereof are segregated together to flow through a common conduit wherein the blood of each is blended with the others before continuing to pass on to the vessels of the opposite party through a confluence, or junction, conduit.

Confluence conduits are described below in section 2b(1)(b)(b14), entitled Confluence Conduits: Connection of One or More Bloodlines to Flow through a Common Channel to the Same or a Different Number of Bloodlines with Self-leveling Apportionment of Outflow, and illustrated in nonexpandable form for use in an adult in FIG. 35A, and switchable confluence conduits are described below in section 2b(1)(b)(b15), entitled Switchable Lines and Confluence Conduits: Long when Intercorporeal, to Short when Intracorporeal, Both Affording Connection of One or More Bloodlines to the Same or a Different Number of Bloodlines with Self-leveling Apportionment of Outflow, and shown schematically in FIG. 35B.

In that both confluence conduits and side-side or ‘pantaloon’ anastomoses often used to combine an accessory with a normal renal artery involve the creation of a common channel, there is some physiological overlap between the two, and the application of this approach is useful for reducing the number of bloodlines, hence, the number of valves needed in a switch transplant, thus reducing the cost of the procedure. For use in a pediatric patient, the material of the conduit is great in tensile strength and can be configured with alternating flats 233 and circumferential expansion vaults 234 as shown in FIGS. 15C, 15D, 36A, and 36F to allow expansion with growth, the constitution thereof specified below in section 2b(2)(f), entitled Expandable Structure to Accommodate Growth and Self-adjust in Size in Vascular Segment Prostheses, Lines, and Confluence Conduits Placed in Children.

Assuming normal vasculature in both donor and recipient, a confluent conduit used to reduce a number of bloodlines as shown in FIGS. 32A thru 32C should spontaneously approximate the normal apportionment in the volume of blood flow delivered into each recipient vessel. That is, even though the outflow of the contributing vessels is combined, provided the same combination of corresponding vessels in the supply or drainage territory within the transplant organ in the donor and recipient are connected, outflow will be self-normodistributed, so that even in an extended metered switch transplant, the blood supply to a given supply territory should prove sufficient. While replacement organs with atheromatous arteries can be angioplastied, this condition would likely disqualify the graft organ for transplantation.

This factor of correct volume distribution by organ supply or drainage territory (spontaneous adaptive flow apportionment, outflow self-leveling, or self-apportioning) from the vessels of a given supply or drainage territory within the transplant organ to that corresponding of the donor or recipient applies not only when flow is between normal patent vessels but when anomalous, such as doubled or abnormally branched vessels in the malformed recipient organ, are connected to the corresponding normal vessels in the other party even when one or more of which are stenosed or atherosclerosed.

That is, at least over the period required to complete a metered switch transplant, if one or more duplicated vessels were stenosed or obstructed and the other patent, provided the supply or drainage territory of the sending and receiving vessels are the same, the sum outflow and inflow between the two should approximate that normal. If flow is to or from an organ, typically, a kidney, with accessory, or supernumerary, arteries, even if one or more of the sending or receiving vessels is stenosed or obstructed, the volume of blood to or from the supply territory of the one kidney should approximate that of the other despite the difference in the number of vessels among which the flow is divided.

Broadly, when a completely normal replacement organ is unavailable, the distribution of flow volume among the vessels of the donor organ should constitute as close to the proper apportionment of blood flow as the malformity of the recipient organ will allow. With respect to the donor organ, where time is available, connection to the corresponding defective vessels of the recipient should not be initiated until corrective measures, preferably transluminal or transcatheteric, have been applied to the defective recipient organ.

By the same token, flow originating from a single normal vessel for outflow into vessels anomalous as duplicated, or bifid, for example, or the reverse should apportion the same sum flow between the two. In switch, or bypass, solid organ transplantation, bypass through the donor organ is by connection of all corresponding vessels between the recipient and donor organs. If an anomalous recipient organ does not present one or more equivalent vessels, or due to a malformity within the recipient organ does not generate the proper outflow, this spontaneous apportionment should still reduce if not eliminate a failure to perfuse the entire parenchyma.

In addition to a foreign body reaction and the need to work against gravity, placement of a second heart in the infrarenal abdomen poses an increased risk of infection by the gastrointestinal microflora, promoting the formation of adhesions around anastomoses and a heart cage if used (see, for example, Bothin, C. G., Okada, M., Midtvedt, T., and Perbeck, L. 2001. “The Intestinal Flora Influences Adhesion Formation around Surgical Anastomoses,” British Journal of Surgery 88(1): 143-145; Bothin, C. G., Okada, M., and Midtvedt, T. 1999. “Postsurgical Adhesion Formation in Germfree and Ex-germfree Rats-A Study Using Three Scoring Scales,” Journal of Investigative Surgery 12(3): 147-150; see also the section below entitled Prevention of Adhesions).

For this reason, positioning in the lower abdomen which avoids disuse atrophy is addressed below in section 2b(1)(b)(b16), entitled Direct Aortocaval Connection: Unified Entry Arterial and Exit Venous Confluence Conduits for the Heterotopic Connection of a Graft Heart in the lower Abdomen. Protection of the graft organ in this location where the ribs do not fully wrap around it is covered below in section 2b(1)(b)(b6), entitled Heart Cage for the Infrarenal Abdominal Positioning of a Second Heart.

For these reasons, placement thus should be resorted to only when alternative placement must be discounted and must be accomplished with accessory channels to allow the automatic release of microbial, inflammatory, and adhesion counteractants by the command microprocessor of the prosthetic disorder response system. Placement at this distance from the mediastinum can also encourage the connection between noncorresponding vessels through one or more confluence conduits.

When one or more confluence conduits are used, one or more druglines can be connected to each confluence chamber, most drug release points conventional, through accessory channels in the servovalves. With pressure leveling valves connected to other vessels and/or servochokes, the use of confluence conduits can dispel the hypovolemia experienced with experimental placement thus in rats used in immunological experiments as addressed below in section 2b(1)(b)(b5), entitled Heterotopic Double Heart Metered Compound Vascular Bypass, or Switched-organ Transplantation [and so on].

For use in a child, whether a neonate, infant, toddler, or young child, both directly and indirectly applied jackets and the insert bridge, or tie-line to which these are mounted are devised to expand with growth and forestall if not avert the need for revision, which prospect in itself poses risks and induces apprehension that degrade the quality of life. If the usual risks were not enough, reentry into the chest may encounter considerable interference due to adhesions, addressed below in this section, subsection entitled Prevention of adhesions. Ordinary side-entry flow-diverting jackets and valves are produced with a rigid polymeric jacket of polyether ether ketone or gear grade nylon to protect a substrate ductus not protected by bone or deep enough that accidental damage thereto would be highly unlikely.

Such side-entry flow-diverting jackets and valves also surround the ductus within a relatively thick layer of viscoelastic polyurethane foam. Viscoelastic polyurethane foam must be surface treated such as through the application of an outer layer of parylene to prevent it from breaking down. Side-entry jackets and valves can made in different sizes. In valves, the diversion chute sliding mechanism is not amenable to expansion with growth. For use in a small child, the valve is chosen as will best serve in the adult. The problem of long-term accommodation in relation to neighboring tissue is avoided because the valve itself can be externally padded within a breakdown-averting parylene coating, and the level of a valve along the line and the angle to which the valve is rotated are variable. In side-entry jacket or valves this likewise provides expandability for use in young pediatric patients, eliminates abrasive contact with neighboring structures, and affords flexibility. If necessary, a side-entry component can be cushioned by enclosure within a jacket of viscoelastic polyurethane foam coated as indicated.

Where a tie-line to replace a vessel is especially long, it can be provided with virtual endothelial function to the extent that nitrates, for example, can be directly released into the lumen as a slow drip delivered through an accessory channel. Agents to treat the vessel supply or drainage territory such as anti-inflammatoires and antimicrobials can be targeted thus as well. All side-entry, inline coupling, and confluence conduits, for example, are provided with at least one accessory channel. To dispel the atheromatous degeneration that follows the chronic hypoxia of complete enclosure of the adventitia by allowing a normal exchange of gases, the shell and underlying foam in ductus side-entry jackets, valves, and nonjacketing side-entry connectors are liberally perforated from the adventitia to the outer surface of the shell.

As shown in FIG. 36G, for implantation in a prenate or neonate, where the extent of growth will be considerable, the prosthesis of vascular segment replacement is shaped with alternating flats and out pocketing circumferential expansion vaults so that it will spontaneously enlarge in step with the luminal pressure, hence, in step with the growth of the patient. Both aortic prostheses and confluence conduits to be implanted, to include that shorter in a switchable confluence conduit as well as the bloodlines leading into and out of it, are made thus for spontaneous expansion.

In addition to allowing expansion in a growing child, elasticity allows compliance with the pulse as well as adjustability in cross-section when in contact with a neighboring structure. In thoracic and thoracoabdominal embodiments, the prosthesis can be standardized in two sizes of neoaorta and thoracoabdominal aorta, for example, both including their respective branches. FIG. 36F shows a replacement aortic arch with supra-aortic branches to be inserted automatically as a tie-line to be secured at each of its five ends by inline coupling jackets with no interruption in blood flow or bleeding. The pattern of alternating flats 233 and circumferential expansion vaults seen in FIGS. 36A and 36F showing inline coupling jackets indicates that this prosthesis is meant for use in a neonate, infant, or small child, reducing if not eliminating the need for later revision.

Shown in FIGS. 15C, 15D, 36A, and 36F, the expandable fabric is addressed below in section 2b(2)(f), entitled Expandable Structure to Accommodate Growth and Self-adjust in Size in Vascular Segment Prostheses, Lines, and Confluence Conduits Placed in Children. Abdominal aortic embodiments are made with branches to replace various segments along the abdominal aorta. Due to the number of these—which may overlap one or more segments—embodiments to replace the superior visceral branches variously include the coeliac axis, superior mesenteric, inferior mesenteric, middle suprarenals, renals, testiculars, or ovarians; the parietal branches include the inferior phrenics, the lumbars, the middle sacral; and the terminal branches include the common iliacs (see for example, Gray, H. Anatomy of the Human Body, Goss, C. M. (ed.), 1966. Philadelphia, Pennsylvania: Lea and Febiger, page 633).

The configuration is that of an idealized thoracic aorta with supra-aortic branches, a thoracoabdominal embodiment continued an additional distance down the descending aorta as would an endoluminal elephant trunk. For application along the abdominal aorta, a comparable prosthesis with entry, exit, and branches connected by means of inline coupling jackets can be provided in two sizes. The thoracic aortic prosthesis is connected at the ascending aorta and in a thoracic embodiment, somewhat downstream from the left subclavian or if present, an anomalous branch along the descending arch. It therefore spans over the segment of the thoracic aorta—whether singular, bifid, or otherwise anomalous—and its landing zones where aneurysms, a coarctation, or an interrupted aortic arch would be found, as might numerous anomalies in the supra-aortic branches.

That is, the prosthesis embodies the normal form of the anatomy such that when its branches are connected to the native branches in the equivalent, meaning correct or nearly correct position to serve the supply territory intended, and the replacement segment inserted to replace the arch per se, any anomaly or anomalies along the arch will have been replaced by this normal overlay. This should allow the relatively quick and simple correction of the distinct majority of anomalies, a few if any required to be treated through preparatory surgery.

As indicated, when a switchable confluence, or junction, conduit such as that illustrated in FIGS. 32A thru 32F is used, only the shorter of the two confluence conduits to be implanted and therefore required to expand with growth is formed as specified. Donor and recipient brought alongside one another, the longer of the two confluence conduits is removed before closing. Remaining connected to the shorter confluence conduit, the arterial and venous entry and exit line switches, the latter shown in FIG. 32G, are implanted with the shorter confluence conduit. If and when the expandability of the implanted shorter confluence conduit is expended, a longer one is reattached to the line switches and that shorter removed.

Fixed end-inserted diverters such as those shown in FIGS. 31A and 31B require interruption in the flow of blood by clamping and life support through the use of full body circulatory sustainment with a pacemaker-defibrillator to be inserted. In contrast, as schematically depicted in FIG. 37A, suddenly introduced into the line from above, inline coupling jacket-inserted conduit-mounted fixed diverters, shown in FIGS. 36A and 37A, overcome this severe shortcoming but connected to only one outlet, are incapable of switching between either of two outlet lines as can solenoid driven side-entry valves, essential for a sudden compound vascular bypass, or sudden switched-organ, transplant and applications in the lower urinary tract, much less continuously apportion the outlet flow, or effluent, blood or urine between either of two outlet passages as can side-entry servovalves, essential for a metered compound vascular bypass, or sudden switched-organ transplantation.

Stump-end to stump-end connectors such as end-inserted connectors such as those shown in FIGS. 31A thru 31C and inline coupling jacket-secured tie-lines such as those shown in the 36 and 37 series figures are only connectors, which unlike valves and servovalves, are incapable of exercising control over the volume of blood they pass through. End-inserted connectors allow native ductus-to-synthetic tubing connection or the reverse without an increase in external diameter but without contact and pressure compliance or adjustability as regulating flow-through or growing in step with a child patient. A valving function is immediately apparent from the presence of a diversion chute and the bifurcated conformation it supports.

Whereas in druglines, pulsatile function is uninvolved, end-inserted connectors can be made of a strong plastic such as gear grade polyamide, or nylon as well as the usual polypropylene of polyetheretherketone. Elastic materials that increase in resistance to further stretching are not used in the expansion structure-based tie-lines described below in section 2b(2)(f), entitled Expandable Structure to Accommodate Growth and Self-adjust in Size in Vascular Segment Prostheses, Lines, and Confluence Conduits Placed in Children.

Infrequent in occurrence, the same is suitable for connecting an artery coursing amid skeletal muscle to and from a catheter—what in generic terms represents a simple type of tie—or bridging line. Application thus would not, as would ordinary side-entry connectors, result in protrusive, abrasive, erosive, and painful sideways encroachment upon neighboring tissue without the need to excise some of the adjacent muscle tissue. Where end-inserted connectors are noncompliant, the thick foam lining in inline coupling jackets will yield to external pressure, as well as comply with the pulse and an increase in blood pressure, although this pertains to only the short segment or segments containing the connector or connectors.

End-connected junctions do not protrude but necessitate transecting the vessel to insert, which requiring interruption in the flow of blood, violate a central object here of gaining access to vascular lumina without having to stop the flow of blood. Passage of the pulse from a native vessel and into a relatively more rigid-walled catheteric bloodline than one native, the pulse continues to propagate as more constrained to the linear and less radial than when passing through an elastic artery, for example; however, upon exiting the catheteric bloodline and reentering the same vessel without an alteration in its physical properties, the pulse will resume its original form, or if entering a different vessel, the form of the pulse will be set by the physical properties of the newly entered vessel wall.

After the simple, or tubular tie-line without branches, the second type of tie-line, shown in FIG. 36F and addressed below in sections 2b(2)(c), entitled Vascular Segment Prostheses: Inline Coupling Jackets and Bridging, or Tie-lines thru section 2b(2)(c)(c3), entitled Types of Inline Coupling Jacket Secured Bridging, or Tie-lines, are referred to as anatomically configured. However, this only means that the prosthesis is intended to replace the vasculature over a specific vessel and includes branches smaller in diameter than the trunk. Eliminating the need to transect and therefore interrupt the flow of blood through the substrate vessel, entry must be as depicted in FIG. 37A from above. Side-entry jackets, valves, and servovalves are capable of being adjusted but cannot duplicate the function of inline coupling jackets.

Inline coupling jacket-secured tie-lines also accomplish connection without the need for cross clamping and local circulatory arrest through the affected segment and thus avoid the serious sequelae which such an interruption can cause as well. However, as section 2b(2)(c)(c2) below, entitled Automatic Insertion into a Ductus of an Inline Coupling Jacket-secured Bridging, or Tie-line and Its Replacement if Necessary, and FIG. 37A are intended to make clear, through gradual exchange of the substrate for the prosthetic line, flow is continuously transferred from the native vessel into the tie-line as the tie-line gradually descends to more and more replace the native vessel so that the relative proportion of blood flow is gradually transferred from the native to the prosthetic line.

Placed as are all inline coupling jacket-inserted tie-lines, to include the replacement of a diseased such as atherosclerotic or missing segment-automatically, as described below in section 2b(2)(c)(c2), entitled Automatic Insertion into a Ductus of an Inline Valve-jacket Secured Bridging, or Tie-line and Its Replacement if Necessary-unless passing amid muscle as to necessitate inordinate dissection, tie-line riding as shown in FIG. 36G can expedite the placement of side-entry devices. That side-entry diversion is critically superior to the end-inserted connectors shown in FIGS. 31A thru 31C in allowing placement without the need to interrupt the flow of blood through the substrate ductus means that not only is connection much less traumatic but that innumerable complications introduced by the apparatus used to sustain this condition are eliminated as well.

As shown in FIG. 36G, if the method for placement of a side-entry device is too awkward, the device can also be placed automatically by mounting it to a short inline coupling jacket-inserted conduit, or segment-bridging tie-line rather than directly to the substrate ductus. Exceptionally, because side-entry devices can be positioned at any level or rotational angle along a vessel, mounting thus allows positioning the side-entry device where the substrate vessel had been diseased or malacotic: by positioning the inline coupling jackets securing the tie-line mounted side-entry device far enough up- and downstream where the substrate vascular tissue is sound, the defective mounting position desired can be excised and replaced with the premounted side-entry device.

If tie-line mounted, multiple side-entry devices of the same or different kind can be placed simultaneously. Also, if multiple units are required, this can become a significant time factor. Placing a side-entry device mounted thus can be completed in less time than if the device is connected directly to the substrate vessel. Reduction in procedural duration is beneficial not only in reducing the stress imposed on the patient, which for a frail patient can even determine whether the procedure should be undertaken, but it also facilitates the completion of a procedure when urgent.

More specifically, rather than to be connected directly to the substrate ductus, side-entry devices from basic ductus side-entry jacket to solenoid- and servomotor-driven side-entry valves can also be produced as already connected to a short inline coupling jacket-inserted conduit (bridge, tie-line) for insertion along the substrate ductus through the automatic procedure described below in section 2b(2)(c), entitled Vascular Segment Prostheses: Inline Coupling Jackets and Bridging, or Tie-lines, which eliminates the need to arrest blood flow by clamping or cardiopulmonary bypass. Accordingly, where FIGS. 1, 2, 5, 7, 8, 10A, 11, 12, 13A, 13B, 14C, 18A, 18B, 19A, 19B, 20A, 20B, 20C, 21A, 21B, 21C, 25A, 25B, 28, 30, and 32A thru 32F show the connection of the side-entry device, valves or servovalves, directly to the substrate ductus, the ductus is to be understood as interchangeable with an inline coupling jacket-positioned tie-line.

Accordingly, whether simultaneously or successively, tie-line mounting can facilitate the placement of side-entry flow diversion jackets, vascular valves, and servovalves, or these different in type, in different locations. Placement thus in a heart transplant where eighteen valves are necessary can considerably reduce the duration of the procedure and therewith the stressfulness on the patient. And it does so over and above the reduction in stress due to the greater surgical, anoxic, and anesthetic trauma and risk associated with a conventional heart transplant. Tie-line inserted servovalves can expedite the placement of side-entry devices of any kind. Broadly, direct side-entry device placement is suitable where the substrate vessel is sound and the number of side-entry type units is readily manageable, which in almost any practical surgical application will be the case.

Provided the substrate vessel is sound, direct mounting is preferred; except for an entry hole about equal in diameter to the lumen in internal diameter made in its side, the substrate ductus remain intact. Side-entry valves with solenoid-driven diversion chutes bistable in being switchable between fully open and fully closed, such as that shown in FIGS. 7 and 8 with integral or fused outlet pipeline or extended bloodline exiting through the sidestem 19 expedite the sudden compound vascular bypass, or sudden switched-organ transplantation of a solid organ, addressed below in section 2b(1)(a), entitled Sudden Compound Vascular Bypass, or Sudden Switched-organ, Transplantation especially of the heart, which as indicated in FIG. 16, requires eighteen servovalves or if the supra-aortic branches are separately jacketed, then twenty-one.

In contrast, a gradual, or metered, compound vascular bypass, or metered switched-organ heart transplant allows the transfer of the graft organ between the circulatory system of the donor and recipient to benefit through a gradually induced intrinsic responsivity rather than a sudden acclimation-preclusive immunological confrontation. What might be called an immunological abrupt or startle reflex is reduced in intensity to lessen what otherwise would likely be an unstudied or blind automatic rejection response through the administration of supplementary microchimerizing immune tolerance-inducing drugs made possible by the direct targeting through servovalve druglines into each donor-recipient connecting bloodline. However, using confluence conduits such as shown in FIG. 35B the number of intercorporeal blood connections can be reduced to two or three, a drugline to each targeting this medication with or without concurrent doses administered systemically to both the donor and the recipient.

FIG. 15A summarizes the reciprocal cross-circulation connections between corresponding vessels of the donor and recipient used to transfer an organ, here the heart, from the circulator system of the donor into that of the recipient. The rate of organ transfer from the donor to the recipient is based on rejection analyte detection by implanted sensors signaling the master control microprocessor of the implanted prosthetic disorder control system executing its prescription-program. The rate of cross flow is controlled by the system master control microprocessor in response to rejection analyte sensor feedback.

Such sensors are positioned within the circulatory systems of the donor and recipient. Rejection analyte sensors are addressed below in this section, in the subsection on sensors, paragraph e. This feedback is used by the controller to regulate the cross flow of blood by the vascular servovalves, that is continuously control the extent and rate of diversion chute travel, hence the mutual acclimation and transfer between circulatory systems.

Whether to replace a diseased or malformed ductus, a segment thereof, or consisting of a straight tube with a tap-off branch to serve as a means for unvalved passive or unregulated diversion of flow, or a great vessel prosthesis, all inline coupling jacket-inserted conduits for use in pediatric patients are constituted and shaped as described above in this section, and below in sections 2b (2)(f), Expandable Structure to Accommodate Growth and Self-adjust in Size in Vascular Segment Prostheses, Lines, and Confluence Conduits Placed in Children, and 6, entitled Description of the Preferred Embodiments of the Invention. The material and structure described there are intended to eliminate the possibility of a rupture while expanding in step with growth. This even in a neonate despite the fast rate of growth, thus eliminating a later need for revision.

The other two types of flow diversion components comprise side-entry, or perivascular collar diversion jackets. All are connected directly to the ductus itself or to an inline coupling jacket-placed tie-line inserted along the substrate ductus as a segment mounting the side-entry valve or servovalve to divert flow perpendicularly or off-perpendicularly into or out of the substrate line. Regardless of the means of connection, fixed diverters are not adjustable remotely or automatically in response to sensor-registered conditions and differ from cut end-inserted diverters such as shown in FIG. 31A in allowing placement without interruption in blood flow. The upturned hooks used to prevent migration of the device are likely to score the endothelium or urothelium when inserted. To avert a reduction in retentive effect, the diverter can be rotated slightly. Straight diverters are used to secure transplantation unassociated anastomotic bypasses.

Along with all the devices addressed herein, both types provide at least one accessory channel, or sideline, to allow the direct delivery, or pipeline-targeting, into the jacket and prosthetic shunt or bypass it connects of medicinal or maintenance agents. These might be anti-inflammatory, antimicrobial, thrombolytic, or crystal dissolving, for example, but not likely an immunosuppressive associated with organ transplantation, which requires valves for adjustability.

Sudden compound vascular bypass, or switched-solid organ transplantation, addressed below in section 2b(1)(a) of like title, or sudden switched-organ, transplantation, requires solenoid-driven all-or-no flow diverting valves, whereas metered switch organ transplantation, addressed below in this section and described in section 2b(1)(b), entitled Metered Compound Vascular Bypass, or Metered Switched-organ, Transplantation, requires continuously variable servovalves. Fixed diversion jackets do not allow switching much less the controllable apportionment of blood through each vessel to and from the donor and recipient organs.

Solenoid-driven all-or-no flow diverting valves are reversible where fixed diversion jackets and inline coupling jackets, described below in section 2b(2)(c1) of like title are not. Accordingly, for sudden compound vascular bypass, or sudden switched-organ transplantation, the scheme depicted in FIG. 16 can be supported by solenoid valves which are reversible and confronted with some exigent circumstance that recommends ending the procedure, allow this with both donor graft and recipient native organs intact to allow postponement, or by inline coupling jackets, which like solenoid valves, can be synchronized but are not switchable or reversible as to allow aborting the procedure.

The foregoing reservations kept in mind, lines fed into an individual long to short or the reverse switchable bloodline, addressed below in section 2b(1)(b)(b13), entitled Bloodline and Drugline Switch Devices: Long when Intercorporeal to Short when Intracorporeal to Accommodate Growth and shown in FIGS. 32A thru 32C; or a switchable confluence conduit, addressed below in section 2b(1)(b)(b14), entitled Confluence Conduits: Connection of One or More Bloodlines to Flow through a Common Channel to the Same or a Different Number of Bloodlines with Self-leveling Apportionment of Outflow, and shown in FIG. 35A; and the confluent lines of long-to-short or the reverse switchable confluence conduits, addressed below in section 2b(1)(b)(b15) can be connected to their source and destination vessels by either side-entry or inline coupling jackets or by some combination of these.

Blood flow channeled through and intervening device such as a confluence conduit can be directly pipeline targeted for the release of medication through one or more accessory channels at 1. The source jacket, 2. Along the individual delivery line into a confluence conduit, for example, 3. As blended along the confluence conduit, for example, or at 4. Any of all destination jackets and the vessels these connect. During a switch organ transplant, this versatility makes possible a capability for the discretionary targeted release of immunosuppressives, anti-inflammatoiremicrobials, and any other hormones, enzymes, agents, or drugs. Thus, the interposition of a confluence conduit imparts to inline coupling jackets switching capability but not adjustable apportionment of flow among bloodlines.

The second type of side-entry perivascular collar diversion couplings, solenoid-driven flow diversion valves, are bistable, that is, either fully retract the diversion chute to let blood or urine pass through the substrate ductus unaffected, or fully extend the diversion chute to pass all flow into another ductus or a synthetic shunt or bypass. Solenoid-driven flow diversion valves are suitable for sudden compound vascular bypass, or sudden switched-organ, transplantation and for flow switched between the native ductus and a prosthetic bypass thus affording an alternate route for flow were the alternative path to become impaired by stenosis, vasospasm, or a mechanical malfunction, for example.

Neither fixed nor bistable diversion couplings can support a switch heterotopic double heart transplant, which requires metered switching and an end-point for the apportionment of blood around halfway between the two hearts as determined by their relative absolute stroke volume. The solenoid cannot execute the fine apportionment during metering, nor can it adaptively adjust the intermediate setting along its throw. Moreover, since such a transplant does not allow anastomosis of the donor heart, neither could solenoids make it possible to switch between the anastomosed and prosthetic paths were the other to become impaired or require routine maintenance. Moreover, while one can produce a solenoid that is also able to stop at the half-way point, that solenoid would still be incapable of fine adjustments to that setting.

The third type, servovalves, are those most versatile. FIGS. 15 and 16 show the connections between corresponding vessels of the donor and recipient to perform a heart transplant using the sudden or metered switch method. When urgent, the operation is accomplished using the sudden compound vascular bypass, or sudden switched-organ, transplantation method with solenoid-controlled valves able to fully extend or retract the diversion chute but not to move to any intermittent degree of extension or retraction commanded to adjust the relative proportion of flow between the shunts or bypasses connected thereto.

Essential to perform a metered switch solid organ, here, a heart transplant-described in greater detail below in Sections 2b (1)(b), entitled Metered Compound Vascular Bypass, or Metered Switched-organ Transplantation, and its subsections, FIGS. 15 and 16 would be implemented using continuously variable side-entry servovalves, which allow the gradual transfer of blood flow from the recipient through the graft organ until the flow consists entirely of the blood of the recipient as having been admixed with the blood of the donor during the gradual switch between circulatory systems of the recipient and donor to put the graft organ in the circulatory system of the recipient, the graft then physically transferred into the recipient.

The donor and recipient having been matched and immune tolerance induction initiated previously, the gradual blending of recipient and donor blood as the organ is transferred from donor to recipient is intended to achieve an additional measure of tolerance induction concomitant to and in order to reduce the intensity of an immune reaction. Otherwise, the same process may provoke an adverse reaction intense enough to signal the need for additional immunosuppressive medication. When such a transplant is administered by a totally implanted prosthetic disorder response system, prepositioned sensors detect analytes associated with rejection and signal the master control module to pipeline-target immunosuppressives directly to the inflamed site automatically in accordance with its prescription-program, which if interactive, can also adjust the concurrent release of other drugs.

FIGS. 25A and 25B shows a set of vascular valves-enlarged for visual clarity-combined to serve as a bypass device for performing a carotid endarterectomy with no interruption in the flow of blood to the brain. Addressed in greater detail below in section 2b(2)(g), entitled Ischemia and Hypoperfusion-free Carotid Endarterectomy Bypass and Prosthesis, conventional shunting in open carotid endarterectomy risks hypoxia induced cognitive impairment and transluminal repair risks tears and the release of embolizing debris. When opened to remove plaque, the flow of blood through the vessel has been diverted through a bypass as to continue without interruption. Incorporating at least one accessory channel, each valve is allowed to remain in place following the procedure to allow the follow-up targeted release of medication into the common and/or the internal carotids.

In FIGS. 25A and 25B, the use of servo-rather than solenoid-driven valves able to switch between the native and prosthetic paths, both providing pulsatile flow, indicates that in this location, the smooth motion of servovalves is preferrable to any abrupt snapping, however damped, of solenoids. The benefits of an alternative flow pattern past vasculature just having been repaired and warranting close follow-up is analogous to those pertaining to the retention of bloodlines to circumvent anastomoses following compound vascular bypass, or sudden switched-organ solid organ transplantation as shown and addressed in conjunction with FIGS. 22A and 22B.

Also, where solenoids are bistable in limitation to the zero or full diversion of flow, the condition of the native path may make it preferrable to reduce the blood pressure through the native path by passing only a proportion of the flow through the synthetic path without a loss in pressure or pulsation at the level of the common outlet. As depicted in FIGS. 25A and 25B, the direct release of medication into the internal carotids can respond to any condition that releases acute episodic or eruption prodroma-diagnostic analytes detectable by sensors along the internal jugulars, for example. The compresence in the elderly of carotid obstruction and psychiatric disorders is relatively common.

FIGS. 22A an 22B show the formation of arterial and venous bypasses in a compound vascular lung transplant following excision of the native lung of the recipient. With a fully implanted automatic prosthetic disorder response system, the arterial and venous bypasses create options not otherwise available. For example, should the native path become or remain diseased or the bypass become obstructed, infected, and/or become overlain with a biofilm, flow can be switched to the other path, and either path can be used to free the other for diagnosis or treatment until a cure is reached.

This ability makes it possible to evade a serious complication that would otherwise interfere with, perhaps even prevent, healing; the disorder response system can be programmed to detect the need to switch to the alternate passage and initiate the release of an antimicrobial, for example, into it. In this way, the system creates a new active means for expediting healing that heretofore would require at least the surgical if endoscopic creation of a bypass. If the problem is chronic and/or intermittent, allowing the bypass to remain after the operation makes it available for use indefinitely.

When incorporated into a prosthetic disorder response system, sensors for biomarkers or analytes indicative of disease or malfunction can signal the implanted microcontroller or master control microprocessor to effect such switching between pathways automatically to initiate therapy well before the ambulatory patient even becomes conscious of the condition. In this way, a malfunction that could result in serious injury or even kill the patient is averted immediately, possibly, without the patient even becoming aware of it until later. In accordance with its prescription-program, the system microcontroller, or in multimorbid disease, the master control microprocessor, can command the direct pipe-targeted delivery to the site of the remedial agents prescribed in the controller program at the same time that it signals the clinic of an emergency and specifies its nature.

Such function is especially important for patients such as infants, adults with a communication disorder, and the mentally impaired (see, for example, American Speech-Language-Hearing Association [ASHA] 1993. “Definitions of Communication Disorders and Variations,” Online at https://www.asha.org/policy_rp_1993-00208/#:˜:text=A %20communication %20disorder; ASHA Supplement. 1993 35(3 Supplement 10): 40-41). When the bypass is connected at the inlet end by a vascular servovalve, the flow through the native vessel can be divided between it and the bypass in any relative proportion. This allows a lower volume of blood to flow through the affected passage to distribute the medication released into it through a drugline, and if the native vessel, to wet the endothelium.

As mentioned above, inline coupling jackets provide a means for end-to-end joining organic and synthetic pipelines with little sideways protrusion but at the cost of omitting switching capability. In this regard, it should be specified why a carotid endarterectomy is not treated in the same way as is the replacement of a segment of the ascending aorta and/or the aortic arch by combined excision and automatic insertion of the prosthetic replacement by inline coupling jackets. The reason is that the aorta should be replaced only when it is so defective that to reconstruct it poses considerable risk and the result would not provide the durability desired, when a reaneurysm or stenosis could follow, nor if as usual, the patient is a newborn, could the reconstruction accommodate rapid growth.

Even in an adult, eliminating the possibility of a rupture or dehiscence following reconstruction makes replacement critically superior. In contrast, the carotids almost always remain structurally normal but defective due to the accumulation of atherosclerotic plaque with age. The remedy is therefore for remove the plaque, although the conventional procedure uses shunting that risks anoxia which the procedure described below in section 2b(2)(g), entitled Ischemia and Hypoperfusion-free Carotid Endarterectomy Bypass and Prosthesis, eliminates.

Since the native segment or the thoracic or thoracoabdominal aorta cannot remain, to switch between it and a prosthetic bypass in order to gain the advantages this would offer such as in relieving the native structure of stress while it heals and retaining the advantage of the chemo- and baroreceptive function as needed of the aortic bodies (see, for example, Goss, C. M. 1966 (ed.). Gray's Anatomy of the Human Body, Philadelphia, Pennsylvania: Lea and Febiger, pages 598, 947, 1010, and 1350) is not an option as it is with the carotids and the carotid bodies (Goss, C. M. 1966 (ed.). Gray's Anatomy, pages 582, 898, 945, and 1351).

In an adult, it may be possible to preserve the aortic bodies during open reconstruction of the thoracic aorta; however, this should justify favoring reconstruction over replacement only when there is a reasonable degree of confidence that the repair will endure, which using current methods of repair as addressed below in section 2b(2)(c), entitled Vascular Segment Prostheses: Inline Coupling Jackets and Bridging, or Tie-lines, documents, is less than fully dependable. Provided the supra-aortic branches are uninvolved, endovascular, or thoracic endovascular aortic repair (TEVAR) allows these to be preserved. Replacement of the aortic arch with the tie-line shown in FIG. 36F results in loss of the aortic bodies but leaves the carotid bodies intact.

Otherwise, the loss in baro- and chemoreception due to the loss of these receptors is generally not considered worth the risk of a rupture likely to prove fatal (see, for example, Igiebor, O. S. and Waseem, M. 2023. “Aortic Trauma,” Online, Treasure Island, Florida: StatPearls Publishing Co.; Lotfollahzadeh, S., Seligson, M. T., and Marx, W. H. 2023. “Aortic Rupture,” Online, Treasure Island, Florida: StatPearls Publishing Co.). The aortic bodies retained, as addressed below in section 2b(2)(g), entitled Ischemia and Hypoperfusion-free Carotid Endarterectomy Bypass and Prosthesis, the loss in carotid body hypoxia chemoreception is effectively compensated for over time.

In a neonate (Sullivan, J. S. and Alafita, T. N. 2021. “Fundamentals of Gas Exchange and the Assessment of Oxygenation and Ventilation,” page 176 and Zaritsky, A. L., Yannopoulos, D., and Nadkami, V. M. 2021. “Regional Circulations,” page 390 and Gutierrez, J. A. Theodorou, A. A. 2021. “Oxygen Delivery and Oxygen Consumption in Pediatric Critical Care,” pages 38-40, 51 in Lucking, S. E., Maffei, F. A., Tamburro, R. F., and Zaritsky, A. L (eds.) 2021. Pediatric Critical Care: Text and Study Guide, Cham, Switzerland: Springer Nature Switzerland), reconstruction rather than replacement with a prosthesis with a high terminal bursting force, in functional terms, the absence of any susceptibility to rupture, of the severely aneurysmal thoracic aorta due to a connective tissue mutation or a severely interrupted thoracic arch should not even be considered. Lost at the outset, autonomic sensation is all the more compensated for. In contrast, the carotids can be repaired with the carotid bodies preserved and little concern for later dehiscence. Preservation of the carotid bodies rather than loss of their autonomic oximetric chemoreception due to replacement by a prosthesis is plainly warranted.

Moreover, since the arrangement of vascular valves shown in FIGS. 25A and 25B is used to allow anoxia-free carotid endarterectomy, it has already been put in place during the repair and requires only not to be removed thereafter. Left in place, the prosthetic disorder response system can switch flow between the native and prosthetic structures to best reconcile sensation with healing and respond to any later complications, although given that the valves incorporate accessory channels to allow the direct pipeline targeting of drugs into the carotids, the prospect of complications is virtually eliminated. This factor in itself warrants retaining the prosthesis in a switching capacity.

Whether delivered through the accessory channels of a carotid prosthesis, or where no endarterectomy had been needed, basic side-entry jackets, medication to counteract such conditions include, for example, antiepileptics, anticonvulsives, antipsychotics, antidepressants, anxiety disorders such as panic, phobic, and stress disorder suppressives, and upon realization, drugs that will suppress progressive degenerative diseases of the brain, or dementias. When released directly into the internal carotids rather than by arterial infusion or intravasation into the systemic circulation, dosage levels of opioids, anesthetics, clonidine, and/or baclofen, for example, to suppress the effects of complex regional pain syndrome, for example, can not only be optimized as posing no risk of side effects, but drastically reduced.

For the control of blood pressure through the native path, a more refined ability to apply fine adjustments in this apportionment rather than limitation to absolute path-switching is essential. The ability to switch flow between the native and synthetic paths allows diagnostics and therapy to be performed on the native path or maintenance on the synthetic path without interruption in blood flow. Where the native vasculature cannot be retained, a similar arrangement can be placed as a permanent prosthesis.

The chief advantages in side-over terminal or end-entry, or insertion into the cut end of a ductus, usually a vessel, to redirect its flow, such as pertains to the device shown in FIGS. 31A thru 31C, are avoiding the need to transect the substrate ductus downstream to the device; the ability to valve the diversion; and above all, elimination of the need to interrupt flow to place the jacket, the latter a central object motivating the other devices shown.

The three kinds of side-entry couplings are in turn one of three type couplings of which those side-entry and inline allow placement without interruption in flow through the substrate ductus. Amid compact anatomy such as that in the region of the great vessels, sideways protrusion is a deficit readily ameliorable through the measures specified above in this section. However, switching and flow apportionment between the substrate ductus and a synthetic shunt require the device at the source vessel rather than through some intervening device.

Arterial and venous entry and exit line switches such as addressed below in section 2b(1)(b)(b13), entitled Bloodline and Drugline Switch Devices: Long when Intercorporeal to Short when Intracorporeal to Accommodate Growth, the latter shown in FIG. 32G, allow such action between either of two synthetic shunts but not between the native ductus and a synthetic shunt, and for the purpose of switching from a longer to a shorter bloodline. As non-side-entry devices, inline perivascular collar couplings make possible the insertion of prostheses to replace a segment of a ductus to allow flow to continue from a native into a synthetic ductus and/or the reverse without sideways diversion and protrusion and without interruption in blood flow during placement; however, these do provide direct switching at the source vessel.

That both type jackets allow placement without interruption in blood flow through the substrate vessel is of cardinal benefit in allowing major repairs in the circulatory system without the need for cardiopulmonary bypass or extracorporeal membrane oxygenation, circulatory clamping and arrest as central factors in neurological complications that arise during an operation, as well as eliminating the time to achieve patient hypothermia before and rewarming after the operation per se has been completed. In the applications presented herein, full body circulation is maintained throughout the procedure.

This disallows the use of conventional cardiopulmonary bypass which arrests circulation through the heart and lungs to allow conventional surgical procedures to be applied to these organs, but rather with sustainment of circulation throughout the body with the aid of a pacemaker-defibrillator. The potentiality of cardiopulmonary bypass, for example, to cause postoperative complications is mentioned below toward the enc of section 2b(1)(b)(b4), entitled Metered Compound Vascular Bypass, or Switched-organ, Transplantation in an Adult.

Yet another type inline, or cut end-inserted, coupling, shown in FIGS. 31A and 31B, is suitable for use in the lower body where brief clamping of small vessels during placement poses little risk. These neither function as valves nor permit placement without suspending flow through and transecting the substrate vessel. All such couplings, side- or end-entry, can incorporate at least one accessory channel to allow the direct pipeline-targeting to the couplings of maintenance agents such as thrombolytic or crystal dissolving, and to the substrate ductus of medication, such as anti-inflammatory of antimicrobial.

In many if not most locations where the obstruction of a bloodline or drugline would quickly become life-threatening, redundancy includes the use of more than one accessory channel. While most accessory channels are incorporated into the structure of the end-effector, it is even simpler to provide independent druglines and bloodlines routed alternatively to remove complete dependency upon any one particular pathway to the site of the obstruction.

If applied to a sudden compound vascular bypass, or sudden switched-organ, heart transplant-described below in Section 2b(1)(a), entitled Sudden Compound Vascular Bypass, or Sudden Switched-organ Transplantation-FIGS. 15 and 16 would be implemented using solenoid-driven bistable, that is, fully retracted or fully extended diversion chutes unable to be positioned at an intervening setting. In a sudden compound vascular bypass, or sudden switched-organ transplant, donor-recipient matching must be accomplished and immune tolerance induction initiated prior to the transplant. Solenoid-driven valves always incorporate at least one accessory channel. Whether transplanted using the sudden or metered switch technique, never having been allowed to die, the donor organ has never experienced the trauma associated with death.

Death generates a storm of immune system reaction analytes which can intensify the resistance to acceptance of the graft organ by the immune system of the recipient (see, for example, Tisoncik, J. R., Korth, M. J., Simmons, C., Farrar, J., Martin, T. R., and Katze, M. G. 2012. “Into the Eye of the Cytokine Storm,” Microbiology and Molecular Biology 76(1): 16-32). The uninterrupted continuation of normothermic perfusion and oxygenation past death moderates if not eliminates this reaction. Moreover, in the event of any midprocedural adverse eventuality, medical or otherwise, which would interrupt a solid organ transplantation were it conventional, compound vascular bypass, or sudden switched-organ transplantation makes it possible to switch all blood supply and drainage back to or from the intact native to the graft organ at any moment. In contrast, a procedure that involves sewing the front of a donor heart onto the back of a recipient heart, for example, does not allow a ‘bailout’ at any moment which reinstates the integrity of the recipient and donor.

In an adult, the devices described herein have been designed to remain functional to the end of life. Since in the vast majority of cases, acquired diseases such as congestive heart failure pertain to the adult population, the number of those to benefit is large. However, due to the multifold increase in all dimensions of the anatomy during growth from neonate to adult, the younger the patient, hence, the more pronounced and quickly acquired the growth, the more difficult and costly it is to accommodate longer intervals with vascular or compound vascular bypass transplantation related prostheses were these made of ‘dumb’ synthetic materials, which are unadapted to accommodate growth (see, for example, Morales, D. L. S., Scully, P. T., Braud, B. E., Booth, J. H., Graves, D. E. and 3 others 2006. “Interrupted Aortic Arch Repair: Aortic Arch Advancement without a Patch Minimizes Arch Reinterventions,” Annals of Thoracic Surgery 82(5): 1577-1584).

An inherent consequence of this is that while the earlier a congenital defect such as a severely malformed heart is corrected-if necessary, through transplantation—the less will be the adverse impact on lifelong mental and physical development as well as life expectancy. However, unless features are incorporated into the prosthesis that allow long-term sustainability until the need for revision arises, the shorter will be that interval. Nevertheless, even in a neonate, much can be done to extend the term of prosthetic sufficiency.

Addressed below in section 2b(2)(c2), in a prosthetic replacement aortic arch, or aortic neoarch, for a neonate or infant much less a prenate, pliancy facilitates conformity to the compound curve of the thoracic arch as it gradually curves downwards to become the thoracoabdominal, then the abdominal aorta, and elasticity to accommodate growth allows a significant interval for the baby to grow and gain in strength before having to undergo further surgery if necessary, whether pertaining to another malformity or a follow-up operation to replace the device.

The durability of the prosthesis and its extent of expandability should be sufficient to allow the patient to reach adolescence without revision, the ultimate objective being to maintain normal circulation as essential for normal development in general and to avoid the need for revision, ideally, over the course of a normal lifetime.

Durability on this level supported by and accessory channel to allow the direct release into the lumen of medication of any kind but primarily antimicrobials, and clot-preventing substances such as a heparin, warfarin, coumarin, phenprocoumon, direct factor Xa inhibitor, and platelet blockers, with thrombolytic, or fibrinolytic, automatic sensor-responsive drips of recombinant tissue plasminogen activator, streptokinase, or urokinase, released primarily into the vena cavae.

Clot counteractive substances are addressed below in section 2b(1)(b)(b6), entitled Heart Cage for the Infrarenal Abdominal Positioning of a Second Heart. The material and configuration of the fabric specified for use in inline coupling jacket-secured prostheses and tubular tie-lines is addressed below in section 2b(2)(f), entitled Expandable Structure to Accommodate Growth and Self-adjust in Size in Vascular Segment Prostheses, Lines, and Confluence Conduits Placed in Children. Spontaneously self-adjusted to fit the patient, a prosthesis made of this fabric can be used in a patient of any age. Sufficient expandability provided mechanically, the vascular prosthesis expanding in response to the increase in blood flow associated with growth.

The polymers of which the prostheses are made-polypropylene or polyetheretherketone-were selected on the basis of their greatest resistance to resorption and manageable levels of resistance to pathogens and thrombi. Highly plasticized, the expandable structure as exemplified in the neothoracic aortic arch of FIG. 36F incorporates sufficient flexibility to conform to the transverse side to side and anteroposterior curvature of the thoracic aorta when first placed and throughout growth without significant resistance.

The substrate vessel transection mechanisms 231 and 231′, indicated as to position without detail in FIG. 36A and shown as part numbers 209 and 212 in FIG. 36E, are those at the inlet side of the outlet half of the entry jacket, and the inlet half of the exit jacket, that is, the same but in reverse in the exit jacket. As are the others, these surfaces are fixed with a suitable epoxy adhesive, and backed by thick layers of viscoelastic polyurethane foam which run to the opposite ends of the half-jackets. The inlet and outlet halves of both entry and exit inline coupling jackets form complementary, that is, vertically separated half cylinders, the two connected by a rotary joint interposed continuous axle, or spine, running along the side opposite to the side that opens.

Highly damped spring hinges 224 and 224′ in FIGS. 36A and 36F at the rear of only the inner half cylinders along this common axle or spine urges the outlet half to gently but forcibly close when opened. The sharpness of the saw, its speed of reciprocation, and force of descent make it impossible for the substrate vessel to merely be pushed down by it rather than instantly transected. Both entry and exit jackets are positioned so that the interface joining their inlet and outlet halves are vertically aligned to the level of the prospective transection. This places the interfaces as the levels where either end of the native segment will be transected and the prosthesis inserted by rotation into in its place.

The procedure completed, two small locks affixed at the opening margins along the front of the jacket halves diametrical to the hinge mounting axle at the rear are used to keep the jacket halves shut. Typically seen on briefcases and suitcases, these consist of two components, analogously describable as male and female. The halves are made either of metal, the male half equipped with a pawl having a notch, eminence, or hole, which passing a spring-loaded undercutting arm in the female half, becomes engaged so that to unlock the device requires manually retracting the arm against the force of the spring, or are made of plastic in which a click-disk in the male half inserts through a slot in the female half to become trapped when the restorative force of the disk causes it to catch over a prominence.

When manually locked shut using the small metal pawl or plastic click-disk-intromitting type briefcase locks on their front sides, the inlet half of the entry jacket and the outlet half of the exit jacket (the same but reversed) are locked in position, fixing the half-cylinder interfaces at the levels of the cutting razors. The outlet half of the entry jacket and the inlet half of the exit jacket close under the restorative force of the inner half cylinder spring hinges 224 and 224′ as shown in FIGS. 36A, 36E, 36F, 37E, and 37F but only to the extent that the trip will allow. Even though not reciprocating, for the serrated razor to descend all the way onto the upper surface of the ductus would eventually incise a normal, much less a diseased, possibly malacotic, ductus causing bleeding, the more readily if the ductus were pulsatile or peristaltic.

For this reason, the trip limits closure of the cutter-carrying halves of the jackets to a small interval above the upper surface of the native ductus. If the trip were to malfunction when the operator or an assistant positioned the jacket along the native ductus, the descent would not be from the fully open but rather from the position to which the trip had allowed it to descend. Descent of the razor edges onto the native ductus from the fully open position is prevented by repeated test closing and opening before using the jackets. The jackets are always tested in advance of the procedure with duplicate jackets available if necessary.

To prevent the serrated razor edge under the force of the spring hinge from descending into contact with the upper surface of the native ductus, a trip in either jacket half is used to limit closure of the half-jackets to within three millimeters thereof. Thus, from the initially opened position, the jackets close under the force of the spring until stopped by the trips. Increasing the number of embodiments contrary to efficient and cost-effective production, the same jackets are used to treat veins where a forceful passage of intrinsic waves is uninvolved.

The mechanism in the interfaces separating the jacket half cylinders which transects the native ductus and rotates its synthetic replacement into alignment with the native lumen under the closing force of the jacket half spring hinge once the trip is disengaged is shown in FIGS. 36C and 37C. FIGS. 37E and 37F provide a schematized representation of the train of components from the hand control to the serrated cutting razors used to transect the native ductus at either end in the manner of a tiny reciprocating saw. When depressed, the switch atop the hand control with inmate button cell compartment actuates small vibratory eccentric rotor motors of the kind used to signal an incoming call on cellular telephones. The vibratory motors can also be actuated by radio remote control rather than hard-wired.

Interposed between the vibratory motors and the spring-loaded reciprocation track mountings of the razors are electrically nonconducting pins, or jacks, which transmit the vibration from the motors to the razors. The same in size as phono-type pin plugs, or pin jacks, the pins are inserted into plug sockets likewise similar to their electrically conductive equivalents located on the sides of the jackets at the interface between the inlet and outlet halves and thus behind and aligned to the razors. Having been inserted into the sockets, the front ends of the pin jacks come into contact with the razor mountings.

Accordingly, when the operator switches on current at the hand control connected to the vibratory motors, the pins transmit the vibration from the motors to the razor mountings, so that both transect the native ductus simultaneously. Alternatively, a time delay can be used to actuate the outlet razor by the interval it takes blood to flow from the inlet to the outlet jacket. Otherwise, actuation of ductus transection by the serrated razors is precisely the same at either end of the segment to be replaced because the trips to free the damped spring hinges are released and reciprocation of the razors at both ends energized at the same or nearly the same moment. In either case, descent upon the substrate native ductus of the reciprocating razors is essentially simultaneous. Descent of the razors until stopped by the trips is due to the force exerted by the spring hinges.

Since contact of the razors with the native ductus-which due to disease or a connective tissue disorder, for example, may be malacotic as well as passing peristaltic or systolic waves-would result in problem bleeding, this is prevented by trip stops which prevent the razors from further descent until the cutting action is begun. To assure that the razors are kept from contact with the native ductus before the vibratory motors are energized, release of the trips must be simultaneous with the initiation of razor reciprocation.

For this reason, inserting the pin jacks into the sockets alone must not disengage the trips; rather, inserting the pin jacks into the sockets and energizing the vibratory motors are both required to disengage the trips freeing the jackets to continue closing and in so doing transecting the native segment and rotating around its prosthetic replacement. When fully closed, the spring-loaded lock engages securing the jacket in a fully closed position. The jackets are therefore prevented from closing onto the upper surfaces of the native ductus unless the razors are reciprocating.

That is, insertion of the pin jacks into the pin jack sockets on the sides of the jacket halves alone before the vibratory motors are started must not retract the trips, and initiating vibration by switching on the vibratory motors at the hand control thus drives the small serrated razors as tiny reciprocating saws and the cutting action combined with the closing force exerted by the spring hinges effecting the transection of the native ductus concurrent with the movement into alignment with the native ductus of the prosthetic replacement segment just behind the razors of which the mounting backplates at either end prevent an outflow of blood.

The mechanism then transects the ends of the native segment while rotating the prosthetic segment into alignment with the cut ends of the native ductus. FIG. 37A shows how the prosthetic replacement segment is mounted just to the rear of the serrated razors to descend into alignment with the native ductus. This automatic closure of the outlet half of the entry jacket and inlet half of the exit jacket ends with the automatic locking of both halves in the small pawl-intromitting type locks provided therefor. The prosthetic segment in the position previously occupied by the native ductus confirmed by the flow of blood through the prosthesis, the current is switched off, and the pin jacks withdrawn. Having been cut through and replaced, the native segment is retrieved with tweezers or forceps.

It will thus be seen that except for:

1. Testing the jackets before initiating the procedure.

2. Positioning the jackets along the native ductus.

3. Closing the inlet half of the entry and outlet half of the exit jacket.

4. Inserting the front ends of the pin jacks into the pin jack sockets.

5. Pressing the switch to initiate the flow of current to both vibratory motors.

6. Pushing the electrically nonconductive vibratory pin plugs into their sockets.

7. Visually confirming the transfer of blood flow from the native to the prosthetic lumen, and

8. Removing the pin jacks once the prosthetic segment is in position.

The implanted disorder response system not only administers the procedure as to its automatic execution but monitors its progress for complications that might require signaling the operator of a need for assistance and/or prompt the release of medication as necessary. Steps left to be accomplished manually are left relatively nondiscretionary and thus rather different than the midprocedural deliberations that accompany a manually performed procedure.

This factor eliminates much risk and complexity, making it possible for surgeons not vascular or urological specialists to undertake it. Accordingly, where the need therefor is exigent and a specialist is not available, the method and means described make it possible to effectuate a potentially life-saving repair, which in sharp contrast to any conventional, will expand with growth and remain considerably more durable and safer, with the need for revision dispelled for years if not for life.

All jacket shells, bridging lines, and any component requiring to accommodate growth or yield to pressure exerted by the pulse or neighboring tissue can be made a high degree of expandability and high end failure strain, or bursting force. Constituted thus, the sides of the jackets should afford sufficient pliancy to bend without resistance regardless of the location where these are placed within the body. Additional flexibility so that each half bends within its respective length can be gained by elongating each half to increase the moments of force. A significant defect along the thoracic aorta results in inadequate circulation, in hypoxia and the disruption of a normal pulse with laminar, or streamline, flow, and should the baby survive, the development of collateral circulation will not have remedied the potentially fatal condition to which these lead.

As to the importance of compliance with propagation of the pulse; it is now clear from deficits in development that result from the nonpulsatile flow following a Fontan repair of a congenitally malformed heart and the results following placement of ventricular assist devices continued in place over a significant period (references provided in the sections below dealing with either subject) that pulsatile flow is essential for normal development and sustainment. For this reason, the replacement of a malformed thoracic aorta in a neonate or infant is of fundamental importance.

With or without extension past the aortic arch to the descending aorta or the thoracoabdominal aorta in what would exceed the area covered by a conventional elephant trunk or frozen elephant trunk repair, the prostheses shown in FIGS. 36A, 36E, and 37D when provided with the flats and vaults structure provides not only a repair that is rupture-proof but placed in a neonate, will expand with growth and allow pulsatile streamline flow as essential for normal development and tissue maintenance and compliant contact with neighboring structures.

The use of an alternating flats and semicircular cross-section circumferential expansion vault configuration shell pertains solely to tie-, or bridging lines and inline coupling jacket halves which conduct blood, not to the shells about perivascular collars or jackets which encircle the adventitia of a substate native ductus. The outer shell of adventitia collaring jackets are made of polyether ether ketone, gear grade nylon, or another biocompatible tough slippery polymer such as a fluoropolymer, and to dispel atherosclerotic degeneration, are always extensively perforated, or fenestrated, down to the adventitia as addressed in U.S. Pat. No. 11,013,858, entitled Nonjacketing Side-entry Connectors and Prosthetic Disorder Response Systems, U.S. Pat. No. 11,389,171, entitled Integrated System for the Infixion and Retrieval of Implants, and U.S. Pat. No. 11,759,186, entitled Ductus Side-entry Jackets and Prosthetic Disorder Response Systems.

Also shared with other type connectors, to include basic ductus side-entry jackets, diversion valves, servovalves, and nonjacketing side-entry connectors, the entry and exit inline coupling jackets of thoracic and thoracoabdominal aortic prostheses, for example, always incorporate at least one accessory channel to allow the directly pipeline targeted delivery into the device and the ductus of tissue to which it is connected of medication or device maintenance agents. A second accessory channel is valuable in allowing the wetting of the foam or adventitia surrounding the ductus with medication such as anti-inflammatory.

These devices are addressed below in section 2b(2)(c1), and the confluent lines of long-to-short or the reverse switchable confluence conduits to be implanted, addressed below in section 2b(1)(b)(b15), entitled Switchable Lines and Confluence Conduits: Long when Intercorporeal, to Short when Intracorporeal, Both Affording Connection of One or More Bloodlines to the Same of a Different Number of Bloodlines with Self-leveling Apportionment of Outflow, and shown schematically in FIG. 35B.

As to fitting a wide range of potential substrate vessels, or compliance to the anatomy of the individual patient, this is made possible by the combination of relatively thick layer of foam between the adventitia of the vessel and the stretchable outer shell, which allows flexion and a flush fit across a range of vessel diameters, and when placed about an artery, compliance to the passage of systols that might otherwise result in downstream migration along the aorta. For a small adult, the degree of expandability sought following placement of the thoracic, thoracoabdominal, or abdominal neoaorta as a neonate or infant is that expansion will have been adequate to eliminate the need for revision.

Eliminating the need for detailed deliberations over the exact placement and configuration of a prosthesis currently marketed or a custom aortoplasty using conventional methods, such as whether to debranch the supra-aortic arteries and move their origin to a common graft initiated at a level more proximal along the ascending aorta. This is accomplished with a trifurcation graft technique that allows avoiding the excessive handling of arch vessels weakened by disease, and minimizes cerebral ischemia.

Another consideration is whether to allow the extension of the endostent, or stent-graft, portion of a hybrid thoracic aortic repair proximal to a lower number landing zone, and yet another whether to use retrograde or antegrade cerebral perfusion, which despite several indications that antegrade is superior, remains without a definitive resolution to this day. Moreover, both endovascular and open surgical techniques have been impugned as respectively posing substantial risk for degradation and the provocation of a stroke (Coselli, J. S. and Green, S. Y. 2009. “Evolution of Aortic Arch Repair,” Texas Heart Institute Journal 36(5): 435-437).

By comparison, the relatively quick placement of an off-the-shelf prosthesis devised to be placed without the need to interrupt the flow of blood is likely to allow its application without the need for expertise acquired through extensive study and experience, bringing such a repair within the scope of less specialized and promptly available operators. No prosthesis currently available would accommodate growth in a neonate or an infant over a long interval; a central object here is to provide a prosthesis for neonates and infants that will avert the damage that would otherwise ensue were malperfusion due to a severe defect of the thoracic or thoracoabdominal aorta not corrected, and in so doing, significantly forestall if not eliminate the need for further surgery.

Thoracoabdominal (caudal) extension in excess of 7 or 8 centimeters with the type prosthesis described herein also alleviates the specialist of the need for expert deliberations as to the relative advantages and disadvantages of conventional and frozen elephant trunk repair with respect to the abdomen, where additional risks to include the avoidance of spinal cord injury which can result in paraplegia await the operator. This consequence appears to result when the trunk exceeds 7 or 8 centimeters in length at which it is more susceptible to kinking and becoming occluded, probably by constriction imposed by the accumulation of clot surrounding the graft.

Other causes appear to be “prolonged hypothermic circulatory arrest time required to implant the FET [frozen elephant trunk] device and/or the occlusion of intercostal arteries at the level of stent-graft deployment,” (Di Bartolomeo, R., Murana, G., Di Marco, L., Pantaleo, A., Alfonsi, J., Leone, A., and Pacini, D. 2017. “Frozen versus Conventional Elephant Trunk Technique: Application in Clinical Practice, European Journal of Cardio-Thoracic Surgery 51:20-128 with citation to their sources).

Use of the prosthesis depicted in FIG. 36F eliminates much of the intricacies and complications of conventional methods of arch replacement (see, for example, Moula, A. I., Romeo, J. L. R., Parise, G., Parise, O., Maessen, J. G., and 3 others 2022. “The Evolution of Arch Surgery: Frozen Elephant Trunk or Conventional Elephant Trunk? Online, Frontiers in Cardiovascular Medicine (Lausanne, Switzerland) 20;9:999314; Wawak, M., Tekieli, L., Badacz, R., Pieniążek, P., Maciejewski, D., and 3 others 2023. “Clinical Characteristics and Outcomes of Aortic Arch Emergencies: Takayasu Disease, Fibromuscular Dysplasia, and Aortic Arch Pathologies: A Retrospective Study and Review of the Literature,” Online, MDPI [Multidisciplinary Digital Publishing Institute] International Biomedicines (Basel, Switzerland) 11(8): 2207; Acharya, M., Sherzad, H., Bashir, M., and Mariscalco, G. 2022. “The Frozen Elephant Trunk Procedure: Indications, Outcomes, and Future Directions,” Online, Cardiovasc Diagnosis and Therapy (Hong Kong, China) 12(5): 708-721; Mutsuga, M., Banno, H., Narita, Y., Komori, K., and Usui, A. 2020. “Editors' Choice. Which is Superior, the Frozen Elephant Trunk Technique Alone or the Classical Elephant Trunk Technique Followed by Second-stage Thoracic Endovascular Aortic Repair for Extensive Aortic Arch Repair?,” Online, Nagoya Journal of Medical Science (Nagoya, Japan) 82(4): 657-666).

The type prostheses described herein have been devised to avoid the need for other methods of large vessel reconstructive surgery all of which can cause a mid- or post-procedural stroke or a vasospasm due to thromboembolism, an interruption in blood flow, hypothermic circulatory arrest, or any other cause at a rate lower than what endovascular stent grafts can achieve. Moreover, unlike an inline coupling jacket-secured expandable type prosthesis as addressed below in section 2b(2)(f), entitled Expandable Structure to Accommodate Growth and Self-adjust in Size in Vascular Segment Prostheses, Lines, and Confluence Conduits Placed in Children, no form of surgical repair along a major vessel is not time-limited due to growth in a small child with an inevitable need for a painful reoperation (see, for example, Spath, P., Campana, F., Tsilimparis, N., Gallitto, E., Pini, R., and 3 others 2024. “Outcomes of Fenestrated and Branched Endografts for Partial and Total Endovascular Repair of the Aortic Arch-A Systematic Review and Meta-analysis,” Online, European Journal of Vascular and Endovascular Surgery (London, England) 67(1): 106-116; Cao, L., Zhang, H., Ge, Y., and Guo, W. 2023. “Avoiding Stroke in Patients Undergoing Endovascular Aortic Arch Repair: JACC Review Topic of the Week,” Online, Journal of the American College of Cardiology (New York, New York) 82(3): 265-277; Zhang H, Rong D, Liu F, Ge Y, Wei R, and 3 others 2023. “Case Series of Total Endovascular repair of the Aortic Arch with a Modular Inner-branched Stent-graft System: First-in-man Experience,” Online, British Journal of Surgery (Oxford, England) 110(9): 1084-1086; Dueppers, P., Reutersberg, B., Rancic, Z., Messmer, F., Menges, A. L., and 3 others 2022. “Long-term Results of Total Endovascular Repair of Arch-involving Aortic Pathologies Using Parallel Grafts for Supra-aortic Debranching,” Online, Journal of Vascular Surgery (New York, New York) 75(3): 813-823.e1; Li, R. D., Chia, M. C., and Eskandari, M. K. 2022. “Thoracic Endovascular Aortic Repair with Supra-aortic Trunk Revascularization is Associated with Increased Risk of Periprocedural Ischemic Stroke,” Online, Annals of Vascular Surgery (Amsterdam, North Holland, Netherlands) 87:205-212; Brown, J. A., Amaoutakis, G. J., Szeto, W. Y, Serna-Gallegos, D., and Sultan, I. 2021. “Endovascular Repair of the Aortic Arch: State of the Art,” Journal of Cardiac Surgery (Hoboken, New Jersey) 36(11): 4292-4300; Dionne, P. O., Tenorio, E. R., Cajas Monson, L. C., Pochettino, A., and Oderich, G. S. 2021. “Total Endovascular Aortic Arch Repair Using 3-Vessel Inner Branch Stent Graft,” Online, Annals of Thoracic Surgery (Amsterdam, North Holland, Netherlands) 112(1): e27-e31; Konstantinou, N., Kolbel, T., Debus, E. S., Rohlffs, F., Tsilimparis, N. 2021. “Fenestrated versus Debranching Thoracic and Endovascular Aortic Repair for Endovascular Treatment of Distal Aortic Arch and Descending Aortic Lesions,” Online, Journal of Vascular Surgery (New York, New York) 73(6): 1915-1924).

In a pediatric patient, the preservation of normal development is all the more likely with a standardized, nontailored, aortic arch aneurysm, coarctation, or interrupted aortic arch replacement prosthesis, or aortic neoarch, described below in section 2b(2)(c), entitled Vascular Segment Prostheses: Inline Coupling Jackets and Bridging, or Tie-lines, and shown in FIG. 36G. The prosthesis, made in an expandable form described below in section 2b(2)(f), entitled Expandable Structure to Accommodate Growth and Self-adjust in Size in Vascular Segment Prostheses, Lines, and Confluence Conduits Placed in Children (but also usable in adults where the operator manually pulls the device open to the required length), includes the supraaortic branches. For this reason, blood flow to the brain through the carotids and the other fields supplied by the supraaortic branches is likewise uninterrupted.

As shown in FIGS. 15 and 17A., in a heart transplant using the switch, or compound native to graft organ bypass, method, connection of the donor and recipient aortas inherently includes the supraaortic branches so that the brachiocephalic and carotid branches as well as the carotids continuously perfuse the brain. The same figures show the connections between the other major vessels. During an organ transfer between either children of adults, if to connect the corresponding native vessels of the donor and recipient requires longer lines to span the distance between the donor and recipient than would be implanted these longer lines can be simple tubes.

Shorter lines suitable for implantation, preferably incorporating the expandable design features laid out in section 2b(2)(f), are exchanged for the long lines using line switches shown in FIGS. 32A thru 32F which must have been applied at the outset of operation with no interruption in blood flow. It is best that the donor and recipient be positioned side by side as this will eliminate the additional complexity of needing line switches, which must be applied at the outset of operation, and can therefore switch between lines of the same origin and destination with no interruption in blood flow.

For visual clarity, line switching has been omitted from FIGS. 20A thru 20C and 21A thru 21C. FIGS. 18A and 18B and 19A and 19B depict the same operation performed on an emergency basis through the sudden switch method using bistable solenoid rather than metered servovalve driven valves. Once the graft organ has been harvested as depicted in FIGS. 20C and 21C, the donor, who can still provide other organs for the same or different recipients, is eliminated from the process. Arterial flow and the positions of the servovalves in a metered bypass heart transplant are shown for the arterial flow in FIGS. 20A thru 20C and for the venous flow in FIGS. 21A thru 21C.

FIGS. 18A, 18B, 19A,19B, 20A thru 20C, and 21A thru 21C are diagrammatic in representing the stages in the transfer of a graft organ, the heart, from the circulatory system of the donor into that of the recipient, that shown in FIGS. 18A thru 19B in a solenoid-driven sudden switch and FIGS. 20A thru 21D in a metered, meaning gradual and measured, switch using vascular servovalves. Due to the number of vessels involved, a heart transplant using a compound vascular bypass method is considerably more complex than a kidney or liver transplant.

In these figures, the outflow from the heart through the arterial system and the return flow through the venous system of the donor and recipient are shown collectively, or summarized. As diagrammatic, FIG. 20C depicts outflow to the lower body as direct, whereas in actuality, flow to the lower body is due to the fact that the aorta having risen craniad out of the heart actually reverses direction. In FIG. 21D the drawing is a step les diagrammatic in that the venae cavae do return blood to the heart from the upper and lower body separately. FIG. 20C depicts the flow of arterial, and FIG. 21C the flow of venous blood in the final stage of organ transfer.

The native heart of the recipient has been excised, and the graft heart of the donor, having been integrated into the circulatory system of the recipient, removed, or harvested, for placement in the chest of the recipient. At this stage, the diversion chutes, part number 18 in FIG. 10A showing a vascular servovalve of the kind used, have been fully extended, so that the flow of recipient blood, has been closed off from the donor and is now fully contained by to exclusively supply the recipient to include the graft organ. In the figures depicting the final stage in the process, that is, FIGS. 18B, and 20C representing the arterial flow, blood flows out of the heart to the capillaries, while in FIGS. 19B, and 21C, representing the venous flow, blood returns from the venules to the heart thus completing the circular course of the circulation.

For further specificity and clarification, refer to the descriptions for each of these figures below in section 5, entitled Description of the Drawing Figures. Retention of the donor valves so that blood continues to flow through the retained valves of the donor when the heart is positioned in the chest of the recipient is intentional and preferred. This is because to retain these is to concurrently implant components which the fully implanted prosthetic disorder response system will use to directly pipe medication into any valve and the vessel to which it is connected.

Below in this section are listed some of the sensors available to allow the detection and identification of different pathogens and pathological conditions. The preference for retention upon implantation of the servovalves and bloodlines relates to both the accessory channels, which could be but are not made separable from the valve so that hypertension can be treated in situ both medicinally and by tapping off a small volume of blood as will prevent the immediate injury caused by the elevated pressure. Oxygenated blood is generally diverted into a neighboring artery and unoxygenated blood into a neighboring vein, in either case, one preferably larger in diameter. FIG. 22F shows such means where the reference to portal as opposed to any other site of hypertension is exemplary.

The location of each sensor is stored in the memory of the master control microprocessor which is programmed to release responsive medication to that site in accordance with its prescription-program. The condition might be an infection, inflammation, autoimmune response, or an indication of graft rejection, for example, and the medication might be an antimicrobial, anti-inflammatory, or rejection counteractant, for example. If a form of medication not maintained in a drug reservoir implanted in the pectoral region such as an infusion of vancomycin is indicated, the control system can signal the clinic by radio medical telemetry.

For example, as the condition if dated will have resulted in heart failure, pending a heart and lung transplant for pulmonary hypertension with consequent heart failure in a child, unless advanced as to necessitate immediate transplantation, a negligible expansion in a prosthetic pulmonary artery is allowed to resolve with growth while treated medically (see, for example, Cruz-Utrilla, A., Gallego-Zazo, N., Tenorio-Castano, J. A., Guillen, I., Torrent-Vernetta, A., and 10 others 2022. “Clinical Implications of the Genetic Background in Pediatric Pulmonary Arterial Hypertension: Data from the Spanish REHIPED Registry [(in Spanish), registry of patients with pulmonary hypertension],” Online, MDPI [Multidisciplinary Digital Publishing Institute] International Journal of Molecular Sciences (Basel, Switzerland) 23(18): 10433; Taha, F. and Southgate, L. 2022. “Molecular Genetics of Pulmonary Hypertension in Children,” Online, Current Opinion in Genetics and Development (London, England) 75:101936).

Along with the prospect for gene therapy, treatment through the retained valve accessory channels is with topically effective or post-liver-pass converted but otherwise conventional medication directly piped to the site, to include anticoagulants and thrombolytics in thromboembolic pulmonary hypertension, and effusion and edema counteractive diuretics anticoagulants, thrombolytics, effusion counteractive diuretics, high concentration (see, for example, Hu. X., Ding, N., Songchen, W., Wang. R., Chen. J., and 5 others 2023. “Lung Transplantation for Pulmonary Arterial Hypertension: Optimized Referral and Listing Based on an Evolving Disease Concept,” Online, MDPI [Multidisciplinary Digital Publishing Institute] Journal of Cardiovascular Development and Disease (Basel, Switzerland) 10(8): 350; Krowl. L., Anjum, F., and Kaul, P. 2023. “Pulmonary Idiopathic Hypertension,” Online, Treasure Island, Florida: StatPearls Publishing Co.: Humbert, M., Kovacs, G., Hoeper, M. M., Badagliacca, R., Berger, R. M. F., and 24 others 2022. “Guidelines for the Diagnosis and Treatment of Pulmonary Hypertension,” European Heart Journal 43(38): 3618-3731, corrected 44(15): 1312; Ruopp, N. F. and Cockrill, B. A. 2022. “Diagnosis and Treatment of Pulmonary Arterial Hypertension: A Review,” Journal of the American Medical Association (Chicago, Illinois) 327(14): 1379-1391; Mandras, S., Kovacs, G., Olschewski, H., Broderick, M., Nelsen, A., Shen, E., and Champion, H. 2021. “Combination Therapy in Pulmonary Arterial Hypertension-Targeting the Nitric Oxide and Prostacyclin Pathways,” Online, Journal of Cardiovascular Pharmacology and Therapeutics (Thousand Oaks, California) 26(5): 453-462; Mayeux. J. D., Pan, I. Z., Dechand, J., Jacobs, J. A., Jones, T. L., and 4 others 2021. “Management of Pulmonary Arterial Hypertension,” Online, Current Cardiovascular Risk Reports (New York, New York) 15(1) 2; Kondo, T., Okumura, N., Adachi, S. and Murohara. T. 2019. “Pulmonary Hypertension: Diagnosis, Management, and Treatment,” Online, Nagoya Journal of Medical Science (Nagoya, Japan) 81(1): 19-30; see also sections below 2b(1)(b)(b9), entitled Single Living and Single or Double Deceased Donor Metered Compound Vascular Bypass, or Switched-organ, Lung Transplantation; and 2b (2)(h)(h3), entitled The ‘Tuned’ Shunt in the Relief of Pulmonary Arterial Hypertension). High concentration oxygen inhalation usually affords additional relief. Also addressed below in section 2b(2)(h)(h3) is balloon angioplasty in interventional cardiology.

The application of an implanted automatic disorder response system to gene therapy based upon a recombinant virus method is to continuously monitor the process, apply follow-up measures as necessary, and signal a need for intervention. Its utility with respect to the use of nonviral gene therapy through naked DNA injection is more thoroughgoing in comprehending not only follow-up and adjunct therapy but administration of the delivery of the DNA into cells. One mechanism for injecting naked DNA is shown in FIGS. 14 thru 16 in U.S. Pat. No. 11,013,858, entitled Nonjacketing Side-entry Connectors and Prosthetic Disorder Response Systems.

The development of other type means of delivery to implement the insertion of DNA into cells through nonviral means include miniaturized implantable embodiments of a biolistic particle delivery system, or ‘gene gun’ electroporation, or electopermeabilization, cellular sonication, or sonoporation, and magnetofection, with additional methods in development such as employ gene editing, messenger RNA delivery, and the use of silencing RNA gene suppression (see, for example, Gupta, K., Czerminski, J. T., and Lawrence, J. B. 2024. “Trisomy Silencing by XIST [X-inactive specific transcript non-coding RNA transcribed from the X chromosome]: Translational Prospects and Challenges,” Human Genetics (Berlin, Germany) in press, 9 March; Jia. J., Yang, J., Qian, L., Zhou, B., Tang. X., and 4 others 2024. “Controlled siRNA Release of Nanopolyplex [hybrid system for molecular engineering at the nano-scale] for Effective Targeted Anticancer Therapy in Animal Model,” Online, International Journal of Nanomedicine (Auckland, New Zealand) 19:1145-1161; Jiao. L., Sun, Z., Sun, Z., Liu, J., Deng. G., and Wang, X. 2024. “Nanotechnology-based Non-viral Vectors for Gene Delivery in Cardiovascular Diseases, Online, Frontiers in Bioengineering and Biotechnology (Lausanne, Switzerland) 12:1349077; Sun, Z., Ren, Y., Zhu, W., Xiao, Y., and Wu. H. 2024. “DNA Nanotechnology-based Nucleic Acid Delivery Systems for Bioimaging and Disease Treatment,” Royal Society of Chemistry Analyst (Cambridge, England) 149(3): 599-613; Yousefi Adlsadabad, S., Hanrahan, J. W., and Kakkar, A. 2024. “mRNA Delivery: Challenges and Advances through Polymeric Soft Nanoparticles,” Online, MDPI [Multidisciplinary Digital Publishing Institute] International Journal of Molecular Sciences (Basel, Switzerland) 25(3): 1739; Lin. M. and Qi. X. 2023. “Advances and Challenges of Stimuli-responsive Nucleic Acids Delivery System in Gene Therapy,” Online, MDPI [Multidisciplinary Digital Publishing Institute] Pharmaceuties (Basel, Switzerland) 15(5): 1450; Zhang, Q., Kuang, G., Li, W., Wang, J., Ren H., and Zhao Y. 2023. “Stimuli-responsive Gene Delivery Nanocarriers for Cancer Therapy,” Online, Nano-micro Letters (Berlin, Germany) 15(1): 44; Zhu, L., Luo. J., and Ren, K. 2023. “Nucleic Acid-based Artificial Nanocarriers for Gene Therapy,” Royal Society of Chemistry Journal of Materials Chemistry B (Cambridge, England) 11(2): 261-279; Jiang Y. Fan M. Yang Z, Liu X, Xu Z. and 11 others 2022. “Recent Advances in Nanotechnology Approaches for Non-viral Gene Therapy,” Royal Society of Chemistry Biomaterials Science (Cambridge, England) 10(24): 6862-6892; Wang. H., Qin, L., Zhang, X., Guan, J., and Mao, S. 2022. “Mechanisms and Challenges of Nanocarriers as Non-viral Vectors of Therapeutic Genes for Enhanced Pulmonary Delivery,” Online, Journal of Controlled Release (Amsterdam, North Holland, Netherlands) 352:970-993; Ren S., Wang. M., Wang, C., Wang. Y., Sun, C., and 3 others 2021. “Application of Non-viral Vectors in Drug Delivery and Gene Therapy,” Online, MDPI [Multidisciplinary Digital Publishing Institute] Polymers (Basel, Switzerland) 13(19): 3307; Sainz-Ramos. M., Gallego, I., Villate-Beitia, I., Zarate, J., Maldonado, I., Puras, G., and Pedraz, J. L. 2021. “How Far are Non-viral Vectors to Come of Age and Reach Clinical Translation in Gene Therapy?,” Online, MDPI [Multidisciplinary Digital Publishing Institute] International Journal of Molecular Sciences (Basel, Switzerland) 22(14): 7545; Ramamoorth, M. and Narvekar, A. 2015. “Non Viral Vectors in Gene Therapy—An Overview,” Online, Journal of Clinical and Diagnostic Research (Delhi, India) 9(1): GE01-6; Gene therapy in the treatment of pulmonary hypertension has met with some success and appears to hold promise (see, for example, Zhao, M., Liu, J., Xin, M., Yang, K., Huang. H., and 3 others 2023. “Pulmonary Arterial Hypertension Associated with Congenital Heart Disease: An Omics Study,” Online, Frontiers in Cardiovascular Medicine (Louanne, Switzerland) 10:1037357; Dai, L. and Du. L. 2022. “Genes in Pediatric Pulmonary Arterial Hypertension and the Most Promising BMPR2 [bone morphogenetic protein receptor type II] Gene Therapy,” Online, Frontiers in Genetics (Lausanne, Switzerland) 13:961848; Bisserier M. Mathiyalagan P. Zhang S. Elmastour F. Dorfmüller P. and 8 others 2021. “Regulation of the Methylation and Expression Levels of the BMPR2 [bone morphogenetic protein receptor type II] Gene by SIN3a [switch-independent 3a transcriptional regulator] as a Novel Therapeutic Mechanism in Pulmonary Arterial Hypertension,” Online, Circulation (Hagerstown, Maryland) 144(1): 52-73; Rai, N., Shihan, M., Seeger, W., Schermuly, R. T., and Novoyatleva, T. 2021. “Genetic Delivery and Gene Therapy in Pulmonary Hypertension,” Online, MDPI [Multidisciplinary Digital Publishing Institute] International Journal of Molecular Sciences (Basel, Switzerland) 22(3): 1179; Welch, C. L., Austin, E. D., and Chung, W. K. 2021. “Genes that Drive the Pathobiology of Pediatric Pulmonary Arterial Hypertension,” Online, Pediatric Pulmonology (Hoboken, New Jersey) 56(3): 614-620; Southgate, L., Machado, R. D., Graf. S., and Morrell, N. W. 2020. “Molecular Genetic Framework Underlying Pulmonary Arterial Hypertension” Nature Reviews. Cardiology (London, England) 17(2): 85-95; Welch, C. L. and Chung. W. K. 2020. “Genetics and Other Omics in Pediatric Pulmonary Arterial Hypertension” Online, Chest (New York, New York) 157(5): 1287-1295 see also preceding paragraph; Dinari, A., Moghadam, T. T., Abdollahi, M., and Sadeghizadeh, M. 2018. “Synthesis and Characterization of a Nano-polyplex System of GNRs [gold nanorods]-PDMAEA[ly dimethylaminoethyl acrylate]-pDNA [plasmid DNA]: An Inert Self-catalyzed Degradable Carrier for Facile Gene Delivery,” Online, Nature. Scientific Reports (London, England) 8(1): 8112; Aguero, J., Hadri. L., Hammoudi, N., Leonardson, L., Hajjar, R. J., and Ishikawa, K. 2017. “Inhaled Gene Transfer for Pulmonary Circulation,” Online, Methods in Molecular Biology (Totowa, New Jersey) 1521:339-349).

Epigenetic factors and long noncoding RNAS have also been cited as potential therapeutic targets (see, for example, Ranasinghe, A. D. C. U., Tennakoon, T. M. P. B., and Schwarz, M. A. 2024. “Emerging Epigenetic Targets and Their Molecular Impact on Vascular Remodeling in Pulmonary Hypertension,” Online, MDPI [Multidisciplinary Digital Publishing Institute] Cells (Basel, Switzerland) 13(3): 244; Dave, J., Jagana, V., Janostiak, R., and Bisserier, M. 2023. “Unraveling the Epigenetic Landscape of Pulmonary Arterial Hypertension: Implications for Personalized Medicine Development,” Online, BioMed Central Journal of Translational Medicine (London, England) 21(1): 477; Jankowski, K., Jagana. V., Bisserier, M., and Hadri, L. 2023. “Switch-independent 3A: An Epigenetic Regulator in Cancer with New Implications for Pulmonary Arterial Hypertension,” Online, 12(1): 10; Karabaeva, R. Z., Vochshenkova, T. A., Zare, A., Jafari, N., Baneshi, H., and 5 others 2023. “Genetic and Epigenetic Factors of Arterial Hypertension: A Bibliometric- and In-silico-based analysis,” Online, Frontiers in Molecular Biosciences (Lausanne, Switzerland) 10:1221337; Gerthoffer, W. 2020. “Epigenetic Targets for Oligonucleotide Therapies of Pulmonary Arterial Hypertension,” Online, MDPI [Multidisciplinary Digital Publishing Institute] International Journal of Molecular Sciences (Basel, Switzerland) 21(23): 9222; Jin Q, Zhao Z, Zhao Q, Yu X, Yan L, and 3 others 2020. “Long Noncoding RNAs: Emerging Roles in Pulmonary Hypertension,” Heart Failure Reviews (Norwell, Massachusetts) 25(5): 795-815).

The placement of prostheses to replace delimited segments of a vessel such as the aortic arch are intracorporeal rather than intracorporeal as is a bypass heart transplant with its reciprocal cross-circulation. That synthetic arterial bloodlines will not exhibit the pulse as a series of passing bulges associated with elastic laminae has little if any significance. While some turbulent flow may accompany the passage of blood at the junctions between the native and synthetic lines, flow through both types of lines is otherwise laminar, or streamlined, and the pulse as a visible phenomenon seen from without an artery is reinstated once the blood resumes flow through an artery.

More recent ‘off-the-shelf’ aortic arch patches may have a frame that flexes to assist in propagating the pulse but are designed primarily to prevent the seepage of blood through suture holes and the weave, require tailoring to replace the specific defect, and are incapable of expansion to accommodate growth, which is a critical consideration for neonates and infants born with a coarctation or an interrupted aortic arch, and sometimes, due to a connective tissue disorder, a congenital aneurysm, already sickly and ill prepared to undergo an operation (see, for example, Cramer, J. W., Ginde, S., Bartz, P. J., Tweddell, J. S., Litwin, S. B., and Earing, M. G. 2013. “Aortic Aneurysms Remain a Significant Source of Morbidity and Mortality after Use of Dacron® Patch Aortoplasty to Repair Coarctation of the Aorta: Results from a Single Center,” Pediatric Cardiology 34(2): 296-301, also cited above; De Paulis, R., Scaffa, R., Maselli, D., Salica, A., Bellisario, A., and Weltert, L. 2008. “A Third Generation of Ascending Aorta Dacron Graft: Preliminary Experience,” Annals of Thoracic Surgery 85(1): 305-309).

The rapid rate and pronounced extent of growth of a neonate or infant makes off-the-shelf thoracic aorta prostheses unusable and manufacturers do not supply these for tiny patients. That manufactured prostheses for older patients are supplied in increments of 2.0 millimeters shows that the service life for one placed in a neonate would be unacceptably brief, with the need to detain revision more than long enough to permit considerable deficits to accrue in neurological and other functions.

Moreover, despite the termination of growth, since an error of no more than 2.0 millimeters combined with stitched junctions is likely to predispose the repair to the development of a leak, the degree of accuracy expected is in itself contraindicative for use even in an adult. Because it has been devised to spontaneously adjust to the size of the substrate vessel over a significant range of sizes, the prosthetic thoracic aortic arch, or aortic neoarch, shown in FIG. 36F can be used in a neonate without the need for revision for a lengthy period during which the baby can gain the strength to withstand the follow-up procedure. In fact, a small adult might avoid the need for revision altogether.

A clear distinction must be drawn between an outlet or inlet flow switch that connects to two lines and the interpositioning of a prosthesis along a single line. For the latter, an inline coupling jacket connected tie-line or tie-line type prosthesis, such as pertains to an aortic neoarch, for example, described below in section 2b(2)(c)(c4) and illustrated in FIG. 36F rather than a switchable side-entry configuration allows the elimination of a protrusive side stem required for retraction of the diversion chute in a vascular valve. Especially in a neonate with its tiny anatomy, abrasive erosion over time due to encroachment upon one or more neighboring great vessels would result in serious complications.

Accordingly, in a neonate with an unsurvivable coarctation or interruption, for example, a prosthesis to replace a segment along the thoracic aorta is not connected and fixed in position with side-entry jackets. In addition to the protrusion of the sidestem, the non-inline factor, meaning the nonadherence to the diameter and long central axis of the substrate ductus, side-entry devices are used to connect a shunt or bypass and divert flow into or out of it, and if valves or servovalves, to control the volume of flow through the shunt or bypass. However, a prosthesis to replace a defective segment along a large vessel such as the aorta does not involve diversion and does not require adjustable control—it requires an inline conformation with the least obtrusiveness or conspicuousness as possible.

This need is served by an inline coupling jacket-secured tie-line type prosthesis. Inline coupling jackets are described below in section 2b(2)(c)(c1), entitled Inline Coupling Jackets Proper. Among the ductus these can be used to connect at either end, that for placement in a child is addressed below in section 2b(2)(f), entitled Expandable Structure to Accommodate Growth and Self-adjust in Size in Vascular Segment Prostheses, Lines, and Confluence Conduits Placed in Children. The embodiments thereof replace different segments along a vessel to include its branches, for example.

For unbranched segments over a range of diameters from infants up through smaller adults, the flats and vaults structure is dispensed in a roll from which the operator can cut off any length required and manually adjust it to the size needed. Branched embodiments are likewise expandable over a large range of sizes. FIG. 36F shows the circumferential expansion flats 233 and vaults 234 structure along the tie-line prosthesis shaped to simulate the thoracic aorta with supraaortic branches. Conformation thus pertains to prosthetic vessels tubular, or bloodlines, and those anatomical, druglines, and confluence conduits for placement in a prenate, neonate, infant, or young toddler.

In such a young child with a severely congenitally malformed heart or vessel such as the thoracic aorta, a prosthetic replacement that grows with the child rather than a surgical repair despite the skill required to construct it averts mental and physical development due to inadequate circulation without restraining growth as does a conventional repair and does so without the need to reenter with the risks and pain this involves before the repair, such as a three stage heart reconstruction, a patch aortoplasty, or a frozen elephant trunk ruptures.

Even if the foam lining inline coupling jackets (and side-entry devices as well) were not encapsulated within a coating of parylene so that its breakdown is improbable to begin with, the sizes of the inlet and outlet paired jackets and the amount of foam in each is so small that the absolute volume of its toxic breakdown product, 2,4-toluenediasmine, is minute compared to that released by out of date breast implants, and would be far too slight to pose a serious adverse reaction. Furthermore, in a neonate or young child, the relative proportion of the breakdown product would have been proportionally reduced with growth.

Conventional methods for repair of the thoracic aorta, for example, demand, a high degree of experience-based skill in applying repairs that elude pretesting or simplification. Inline coupling jacket-secured flats and vaults prostheses are intended to do away with the inordinate skill and expertise required and make it possible for general surgeons to take on these cases. This means that the emergency repair of aortic arch malformities such as aneurysms can be passed down from tertiary and quaternary medical centers with long waiting times and congestion to local hospitals for prompt action to avert the risk of death. Unlike patch aortoplasty-endostent hybrid repair, an inline coupling jacket-secured prosthesis includes the supraaortic branches and does not call for the debranching of these to allow the repair to be extended along the aorta. This in itself simplifies the operation required.

While less severe cases of interrupted aortic arch and coarctation are survivable, the neonate with a severe case does not have the time to develop collateral circulation as often conceals severe obstruction due to plaque in a coronary artery. The need to initiate normal circulation imperative, the active options are an immediate heart transplant or the repair or placement of the defective arch. However, the odds that a well matched neonate continued on life-support past death with a sound replacement heart could be located and the graft organ delivered in time are poor, and the odds that the operation would be survived indefinite.

The unavailability of a standardized prosthetic thoracic aortic arch that spontaneously adapts over the range of sizes expected in a neonate or infant to obtain the correct fit where an extensive defect refractory to surgical correction makes custom design of a patching and/or endostenting repair necessary is inseparable from inaccuracies in shaping and sizing to which urgency predisposes. This includes the development of leaks, and in a young patient for whom the need for stabilization and the avoidance of repeated operations is greatest, the inability to expand with growth as the usual cause for the development of leaks.

In this circumstance, a prosthetic arch which can be placed quickly without interruption in blood flow to the upper body, without inordinate hands-on experience unlikely to be available, without cardiopulmonary bypass, general anesthesia, clamping, cardioplegia, or hypothermic circulatory arrest necessitating rewarming time, and allows for growth and time to heal beyond that of any alternative approach is far more likely to succeed. Life support in this context consists of full body circulation through the use of a pacemaker-defibrillator. Described below in sections 2b (2)(c) and 2b(2)(e5) is a standardized replacement aortic arch device suitable for production in four pre-nonextended sizes, each in three different lengths for extension into the ascending or thoracic descending aorta as necessary, to accommodate patients of all sizes.

For neonates and infants, that smallest will not only serve to replace the detailed custom tailoring required for patient-by-patient patch aortoplasty and/or endostenting with a manufactured, quality control and torture tested prosthesis suitable for quickly replacing the thoracic aorta or pertinent segment thereof without the need for precise measurements and stitching. It will expand entirely to the adult dimensions while maintaining greater resistance to rupture than can any reconstruction, which is not amenable to expansion to accommodate growth (Sade, R. M., Crawford, F. A., Hohn, A. R. Riopel, D. A., and Taylor, A. B. 1984. “Growth of the Aorta after Prosthetic Patch Aortoplasty for Coarctation in Infants,” Annals of Thoracic Surgery 38(1): 21-25).

When only a small segment of the aorta or other ductus is affected, only that portion should be replaced (see, for example, Al-Husayni, F., Samman, A., Althobaiti, M., Alghamdi, A., and Alkashkari, W. 2021. “Late Complications after Previous Surgical Repair for Coarctation with Extra-anatomic Bypass Graft: Report on Two Cases,” Cureus 13(1): e12722; Pu, X., Wang, M., Huang, X. Zhang, H., and Huang, L. 2021. “Case Report: A Novel Approach for the Emergency Repair of Acute Aortic Rupture Associated with Congenital Aortic Coarctation,” Journal of Cardiothoracic Surgery 16:170. Cureus 13(1): e12722; Rossi, U. G., Lerardi, A. M., Carrafiello, G., and Cariati, M. 2020. “Aortic Coarctation,” Aorta (Stamford, Connecticut). 8(2): 46-47; Beckmann, E. and Jassar, A. S. 2018. “Coarctation Repair-Redo Challenges in the Adults: What to Do?,” Journal of Visualized Surgery 4:76; Lemaire, A., Cuttone, F., Desgue, J., Ivascau, C., Caprio, S., and 3 others 2015. “Late Complication after Repair of Aortic Coarctation,” Asian Cardiovascular and Thoracic Annals 23(4): 423-429; Chen, S. S., Dimopoulos, K., Alonso-Gonzalez, R., Liodakis, E., Teijeira-Fernandez, E., and 9 others 2014. “Prevalence and Prognostic Implication of Restenosis or Dilatation at the Aortic Coarctation Repair Site Assessed by Cardiovascular MRI in Adult Patients Late after Coarctation Repair,” International Journal of Cardiology 173(2): 209-215).

Materially reducing the ability to benefit a neonate, edge-stitiched patch aortoplasties, endografts, and repairs hybrid in including an endoluminal extension, or more specifically, a frozen elephant trunk, are incapable of expanding in response to growth (see, for example, Agasthi, P., Pujari, S. H., Tseng, A., Graziano, J. N., Marcotte, F. and 4 others 2020. “Management of Adults with Coarctation of Aorta,” World Journal of Cardiology 12(5): 167-191). In an aneurysm and/or coarctation or interrupted aorta bypass prosthesis such as that shown in FIG. 36F or in a multiple bloodline, or multiline, confluence conduit for use in an adult such as those shown in FIGS. 15C and 15D, circumferential alternating flats 233 and semicircular cross-section circumferential expansion vaults 234 as shown in FIG. 36A can be used to achieve extendability and compliance to simulate that contributed by the internal and external elastic laminae in the walls of large native arteries (see, for example, Berne, R. M. and Levy, M. N. 1993. Physiology, Chapter 27. “The Arterial System,” pages-453-464. St. Louis, Missouri: Mosby-Year Book).

A failure to accommodate growth after conventional patch aortoplasty, for example, is seen as the development of a significant reduction in right arm systolic blood pressure, hypoplasia of the transverse arch, and the emergence of various aberrations in the diameter of the thoracic aorta. Worse, “Growth of the intact posterior wall of the unresected coarctation after aortoplasty, however, has not yet been demonstrated to occur,” (Sade, R. M., Crawford, F. A., Hohn, A. R., Riopel, D. A., and Taylor, A. B. 1984. “Growth of the Aorta after Prosthetic Patch Aortoplasty for Coarctation in Infants,” Annals of Thoracic Surgery 38(1): 21-25)(see also, Backer, C. L, Paape, K., Zales, V. R., Weigel, T. J., and Mavroudis, C. 1995. “Coarctation of the Aorta. Repair with Polytetrafluoroethylene Patch Aortoplasty,” Circulation 92(9 Supplement): 11132-11136; Rostad, H., Abdelnoor, M., Sorland, S., and Tionneland, S. 1989. “Coarctation of the Aorta, Early and Late Results of Various Surgical Techniques,” Journal of Cardiovascular Surgery (Turin, Italy) 30(6): 885-890).

Conventional methods of repair tend to be susceptible to the subsequent formation of an aneurysm, pseudoaneurysm, or recoarctation (see, for example, Kamada, T., Tanaka, R., Iwase, T., Oyama, K., and Kin, H. 2020. “Successful Ascending to Descending Aortic Bypass and Endovascular Embolisation of a Late Pseudoaneurysm following Patch Aortoplasty for Coarctation of the Aorta,” EJVES [European Journal of Vascular and Endovascular Surgery] Vascular Forum 47:22-25). Beckmann, E. and Jassar, A. S. 2018. “Coarctation Repair-Redo Challenges in the Adults: What to Do?,” Journal of Visualized Surgery 4:76; Cramer, J. W., Ginde, S., Bartz, P. J., Tweddell, J. S., Litwin, S. B., and Earing, M. G. 2013, Op cit. above in this section; Al-Hroob, A., Husayni, T. S., Freter, A., Chiemmongkoltip, P., Ilbawi, M. N., and Arcilla, R. A. 2003. “Aortic Aneurysm after Patch Aortoplasty for Coarctation: Analysis of Patch Size and Wall Growth,” Pediatric Cardiology 24(1): 10-16).

Conventional methods of repair also tend to lack sufficient durability. Over time, such repairs may develop a leak that necessitates reentry to repair. Most significantly for use in an infant, conventional repairs do not expand in step with growth, thus that revision will be needed, notwithstanding the high rate of growth or the impaired condition of the patient. By comparison, a standardized aneurysm and/or interrupted aorta or coarctation bypass replacement prosthesis can be made to not only expand with growth and thus forestall or eliminate the need a revision that often will also lack durability but can be placed without the need to interrupt the flow of blood, which can result in serious complications.

Without cardiopulmonary bypass during coronary artery bypass graft surgery, for example, which provides circulation to the body other than through the heart and lungs, and without cross-clamping, cardioplegia, and general anesthesia, the odds for the development of adverse sequelae is substantially reduced. An improbable desynchronization during the replacement procedure is warded off through the routine use of a pacemaker-defibrillator. In this, frequent electrostimulator shocks can also do injury, so that accessibility greater than when implanted in an ambulatory patient, shocks should be allowed only when necessary and measures taken to reduce the injury that would ordinarily result.

### Anoxia-Free Aortic Repair: Thoracic and Thoracoabdominal Aortic Prostheses

This should eliminate the potentially significant complications each of these measures harbors in addition to the serious adverse sequelae to which patch aortoplasties and endovascular stents, or endostents are susceptible (references provided below in section 2b(2)(d), entitled Anoxia-free Aortic Repair: Aortic Prostheses Thoracic, Thoracoabdominal, and Abdominal, which are standardized inline expandable aneurysm and/or coarctation or interrupted aortic arch replacement prostheses. A common example of a plastic joint, albeit with less restorative force, is seen in plastic bottle caps made of high-density polyethylene where the hinge connecting the cap to its holding ring encircling the bottle toward its top consists of a narrow section continuous with the rest of the cap which is narrowed down to function like a thin elbow.

However, polyethylene and polypropylene are not formulated to exhibit intrinsic elasticity, which limits the expansion to which a prosthesis made of either might attain. Expandability critically extends the usable life of the standardized inline prosthetic aneurysm and/or coarctation or interrupted aorta bypass conduits here advanced beyond that of any relatively inflexible and inelastic device. In a neonate, expandability eliminates the need for revision too soon if ever after initial placement. Since not just each bloodline but the confluence section of a multiline confluence conduit can be made expandable, when placed in the young, these can continue in use over a considerable period.

Trauma in replacing an aneurysm and/or coarctation bypass conduit is much reduced by bypassing the original bypass—that is, waiting for the proximal margin of the inlet diversion jacket along the ascending aorta to grow in length so that the larger replacement inlet diversion jacket can be applied within that previously unaffected segment. Due to the compactness of the great vessels toward the root of the aorta, especially in a young patient, the original inlet bypass jacket will usually be placed distal enough along the aorta—just short the origin of the innominate, or brachiocephalic, about the center of landing zone 0—that subsequent growth will provide the space needed to position a later bypass inlet jacket.

For the correction of congenital malformities, that prostheses can be designed to enlarge with growth means that solid organ transplants and the correction of defects of the great vessels, such as an interrupted aortic arch, coarctation, or transposition of the great vessels can be applied at the outset of life and remain in place for an interval long enough to prevent the global deterioration in development that would otherwise have ensued. That an expandable prosthesis can sustain normal development and eliminate the nee for painful revision is of the utmost benefit.

With a growth-compliant prosthesis, growth hormone transmitted in the blood should assure unrestricted uniformity of growth throughout the vascular tree. With a totally implanted prosthetic disorder response system, druglines inserted into the accessory channel or channels in either or both inlet coupling half-jackets shown as part number 8 in FIGS. 36A, 36E, 36F, 37A, 37B, and 37D before implantation can accept a drugline to treat any medical contingency that might arise and do so immediately, before the patient even becomes aware of the problem.

While the upstream jacket remains in place, healing can be supported through the direct pipeline-targeting of drugs such as immunosuppressive, anti-inflammatory, and antimicrobial to the anastomosis through one or more accessory channels. Once removed, the direct delivery of drugs can be continued by replacement of the servovalves with basic side-entry jackets.

Vascular servochoke-type servovalves such as shown in FIGS. 14A thru 14C are suitable for adjusting a local blood pressure without the need for a vascular servovalve to tap off a certain volume of blood for diversion into another larger artery vein or otherwise constricting the supply artery to render an organ or volume of tissue temporarily hypoxic as much if not more than a malignant tumor within it and therefore less susceptible to injury from abscopal exposure (spillover, overspray, bystander effect).

The inducement of hypoxia in the healthy tissue surrounding a cancer so that the tumor is left more highly oxygenated, hence, more susceptible to radiation (or the reverse, a relative increase in the oxygenation of the tumor) are addressed below in this section (see, for example, Swartz, H. M., Vaupel, P., and Flood, A. B. 2023. “A Critical Analysis of Possible Mechanisms for the Oxygen Effect in Radiation Therapy with FLASH [[Free Electron LASer (soft X-ray free-electron laser) in Hamburg],],” Advances in Experimental Medicine and Biology (New York, New York) 1438:127-133; Swartz, H. M., Hoopes, P. J., Gladstone, D. J., Demidov, V., Vaupel, P., and 4 others 2022. “A Radiation Biological Analysis of the Oxygen Effect as a Possible Mechanism in FLASH [soft X-ray Free-electron LASer) in Hamburg],” Online, Advances in Experimental Medicine and Biology (New York, New York) 1395:315-321; Bouleftour, W., Rowinski, E., Louati, S., Sotton, S., Wozny, A. S., and 4 others 2021. “Review of the Role of Hypoxia in Radioresistance in Cancer Therapy,” Medical Science Monitor (Smithtown, New York) 27: e934116; Rakotomalala, A., Escande, A., Furlan, A., Meignan, S., and Lartigau E. 2021. “Hypoxia in Solid Tumors: How Low Oxygenation Impacts the “Six Rs” of Radiotherapy,” Online, Frontiers in Endocrinology (Lausanne, Switzerland) 12:742215; Kim, S. W., Kim, I. K., and Lee, S. H. 2020. “Role of Hyperoxic Treatment in Cancer,” Online, Experimental Biology and Medicine (Maywood, New Jersey) 245(10): 851-860; Zhou, M., Xie, Y., Xu, S., Xin, J., Wang, J., and 4 others 2020. “Hypoxia-activated Nanomedicines for Effective Cancer Therapy,” European Journal of Medicinal Chemistry (Paris, France) 195:112274; Epel, B., Maggio, M. C., Barth, E. D., Miller, R. C., Pelizzari, C. A., and 6 others 2019. “Oxygen-guided Radiation Therapy,” Online, International Journal of Radiation Oncology, Biology, Physics (New York, New York) 103(4): 977-984; Graham, K. and Unger, E. 2018. “Overcoming Tumor Hypoxia as a Barrier to Radiotherapy, Chemotherapy, and Immunotherapy in Cancer Treatment,” Online, International Journal of Nanomedicine (Auckland, New Zealand) 13:6049-6058; Jahanban-Esfahlan, R., de la Guardia, M., Ahmadi, D., and Yousefi, B. 2018. “Modulating Tumor Hypoxia by Nanomedicine for Effective Cancer Therapy,” Journal of Cellular Physiology (New York, New York) 233(3): 2019-2031; Richardson, R. B. and Harper, M. E. 2016. “Mitochondrial Stress Controls the Radiosensitivity of the Oxygen Effect: Implications for Radiotherapy,” Online, Oncotarget (Albany, New York) 7(16): 21469-21483; Weinmann, M., Welz, S., and Bamberg, M. 2003. “Hypoxic Radiosensitizers and Hypoxic Cytotoxins in Radiation Oncology,” Current Medicinal Chemistry. Anti-cancer Agents (San Francisco, California) 3(5): 364-74, additional references cited below in this section).

In addition to literal, meaning mechanical, directly piped drug targeting as elaborated upon in U.S. Pat. No. 11,759,186, entitled Ductus Side-entry Jackets and Prosthetic Disorder Response Systems, as well as here, more recent methods for the selective targeting of a tumor, for example, to include 1. Superparamagnetic nanoparticle carrier bonded tagging, 2. The use of a significant difference in the level of oxygen uptake as illustrated in FIGS. 14B and 14C and explained in the text, 3. Optogenetic tagging, 4. Docking (synaphic, pathotropic, active targeting), 5. Polymeric-controlled release or elution, and 6. Convection-enhanced delivery (see, for example, Yao, Y., Zhou, Y., Liu, L, Xu, Y., Chen, Q., and 5 others 2020. “Nanoparticle-based Drug Delivery in Cancer Therapy and Its Role in Overcoming Drug Resistance,” Online, Frontiers in Molecular Biosciences Nanobiotechnology Hybrids Part B. Hybrids for Drug Delivery Online at https://doi.org/10.3389/fmolb.2020.00193; Pandey, S., Oza, G., Mewada, A., Shah, R., Thakur, M. and Sharon, M. 2013. “Folic Acid Mediated Synaphic Delivery of Doxorubicin Using Biogenic Gold Nanoparticles Anchored to Biological Linkers” Royal Society of Chemistry Journal of Materials Chemistry Part B. Materials for Biology and Medicine 1(9): 1361-1370; Ruoslahti, E., Bhatia, S. N., and Sailor, M. J. 2010. “Targeting of Drugs and Nanoparticles to Tumors,” Journal of Cell Biology 188(6): 759-768; Sugahara, K. N., Teesalu, T., Karmali, P. P., Kotamraju, V. R., Agemy, L., and 4 others 2009. “Tissue-penetrating Delivery of Compounds and Nanoparticles into Tumors,” Cancer Cell (Cambridge, Massachusetts) 16(6): 510-520; see also references cited below as pertinent to tumor sensors); Sawyer, A. J., Piepmeier, J., and Saltzman, W. M. 2006. “New Methods for Direct Delivery of Chemotherapy for Treating Brain Tumors,” Yale Journal of Biology and Medicine 79(3-4): 141-152).

A prosthetic disorder response system is capable of supporting all of these techniques, as well as future developments in the use for these in various combinations, such as combining oxygen deprivation by means of a servochoke with nitric oxide-releasing nanoparticles (see, for example, Alimoradi, H., Greish, K., Barzegar-Falla, A., Alshaibani, L., and Pittala, V. 2018. “Nitric Oxide-releasing Nanoparticles Improve Doxorubicin Anticancer Activity,” International Journal of Nanomedicine 13:7771-7787) will be no less accommodable.

No measures described here are intended to treat disease that is temporary, or acute, not serious, or routinely treated by conventional means. An automatic disorder response system comprises small drug reservoirs and direct-to-target drug delivering fluid and implanted electrostimulator wired or wireless electrical lines to allow the direct application to the nidi or sites of disease of drugs and/or electrostimulation, imaging probes, and other diagnostic and therapeutic end-devices as necessary under the control of an implanted microcontroller or microprocessor as master controller executing a prescription-program.

Targeting in the present context denotes directly focused topical treatment, not the systemically dispersed pharmaceutical targeting of a certain cell type, for example. All components of the implanted system having been devised for permanent placement, removal during a remission or subsidence is discouraged; rather, the parts are left in place where these can be reassigned endoscopically in relatively short order to any new site of disease that may appear.

### Prevention of Adhesions

Constant surveillance and response by the fully implanted prosthetic disorder response system should reduce postoperative complications to a minimum. However, postsurgical anatomical changes not reflected in the physiological indicia monitored will not be affected much less prevented. Included is the process by which fibrinous exudates produced by inflammation transition into bands, some thick, vascularized, and innervated, often associated with abscesses, granulomas, and hematomas. Based upon the location, adhesions risk bowel obstruction, chronic pain, dysfunction of the urinary bladder, infertility, interfere with reoperation, and other complications. Exclusively harmful, adhesions are never intentionally or strategically stimulated to form in order to stabilize implants, for example.

This can be accomplished by connection of a band produced in response to the findings concerning complications with surgical meshes and connected at either end by a nonjacketing side-entry connector with hollow needles such as shown in U.S. Pat. No. 11,013,858 to release a topical anesthetic such as lidocaine to dispel irritation pending the development of an immune system-stimulated envelopment in a fibrous capsule (see for example, Lu, X., Harman, M., Todd, H. B., Augenstein, V., McIver, B., and Bridges, W. 2022. “Analyzing Material Changes Consistent with Degradation of Explanted Polymeric Hernia Mesh Related to Clinical Characteristics,” Surgical Endoscopy (in press); Reyes, P. B. G. and Hashim, H. 2020. “Mesh Complications: Best Practice in Diagnosis and Treatment,” Therapeutic Advances in Urology 12:1756287220942993; Baylon, K., Rodriguez-Camarillo, P., Elias-Zuniga, A., Diaz-Elizondo, J. S., Gilkerson, R., and Lozano, K. 2017. “Past, Present, and Future of Surgical Meshes,” Membranes (Basel, Switzerland) 7(3): 47).

This same eventuality affects implanted sensors upon which the implanted disorder response system depends, obstructing the line of sight and making the use of coat-forming counteractive measures important. That the components of a totally implanted prosthetic disorder response system are remotely controlled by the system master controller and major vascular prostheses such as an artificial aorta and confluence conduits for placement in a child are made with an elastic and rupture-proof fabric described in section 6, entitled Description of the Preferred Embodiments of the invention, in the subsection entitled Artificial Aorta and Its Automatic Placement to expand with growth without the need for reentry eliminates the need for revision and concern over the need for adhesiolysis or the use of measures to prevent the formation of adhesions.

Sensors prepositioned in locations vulnerable to the development of adhesions will eventually detect the analytes diagnostic for this process, but such exceeds the existing state of the art (see, for example, Zong, X., Li, S., Chen, E., Garlick, B., Kim, K. and 6 others 2004. “Prevention of Postsugery-induced Abdominal Adhesions by Electrospun Bioabsorbable Nanofibrous Poly(lactide-co-glycoside)-Based Membranes,” Annals of Surgery (Philadelphia, Pennsylvania) 240(5): 910-915; Liakakos, T. Thomakos, N, Fine, P. M., Dervenis, C., and Young, R. L. 2001. “Peritoneal Adhesions: Etiology, Pathophysiology, and Clinical Significance. Recent Advances in Prevention and Management,” Digestive Surgery 18(4): 260-273; Thompson, J. 1998. “Pathogenesis and Prevention of Adhesion Formation, Digestive Surgery 15(2): 153-157; Luijendijk, R. W., de Lange, D. C., Wauters, C. C., Hop, W. C., Duron, J. J., and 5 others 1996. “Foreign Material in Postoperative Adhesions,” Annals of Surgery (Philadelphia, Pennsylvania) 223(3): 242-248).

For this reason, adhesion prevention is no less important in compound vascular bypass heart transplantation and the correction of extracardiac transposition of the great vessels than it is in conventional cardiac surgery. Moreover, critical in orthotopic procedures where the heart remains in the anatomical space provided for it, the avoidance of adhesions in double heart compound vascular bypass transplantation, where the added heart is positioned to a side of the innate heart or in an inferior location where the anatomy was never intended to accommodate it and more liable to develop adhesions is even more important.

Not limited to thin bands of connective tissue, adhesions are often thick vascularized and innervated bridges of tissue fused at either end which pose a considerable hindrance to obtaining a clear field. No less problematic, following adhesiolysis, or division, adhesions display a strong tendency to reform (Diamond, M. P. and Freeman, M. L.2001. “Clinical Implications of Postsurgical Adhesions,” Human Reproduction Update 7(6): 567-576). Such pericardial-stemal adhesions, for example, can make access to the underlying heart difficult, considerably extending procedural durations.

For these reasons, to eliminate adhesions may require extensive resection, encouraging the retaining of the defective heart and instead assisting it by means of placing a second heart, in the abdomen with vessel-to-vessel connections to the native heart as addressed below in section entitled 2b (1)(b)(b5), entitled Heterotopic Double Heart Metered Compound Vascular Bypass, or Switched-organ Transplantation [and so on], or the added heart, placed by direct aortocaval connection is allowed to function independently of the native heart, or is connected to the native heart.

Section 2b(1)(b)(b16) below, entitled Direct Aortocaval Connection: Unified Entry Arterial and Exit Venous Confluence Conduits for the Heterotopic Connection of a Graft Heart in the Lower Abdomen, and section 2b(1)(b)(b17), entitled Doubly Heterotopic Double Heart Compound Vascular Bypass, or Switched-organ Transplantation, and 2b (1)(b)(b18), entitled Hybrid Doubly Heterotopic Double Heart Transplantation, describe other schemes for connecting the donor to the recipient heart. Every case unique, these different connection schemes might be further elaborated in terms of the automatic release of inotropes, heart supportive, and anti-atherosclerotic medication by the monitoring prosthetic disorder response system, which can supervise and control endovascular pumps, and cooperate with pacemakers and cardioverter defibrillators, for example.

With a second heart in the abdomen, vessel-to-vessel connection with the native heart and the application of these exogenous means to achieve pulmonary sufficiency is the most pressing object. Positioning a second heart in the abdomen eludes any condition that would preclude placing an orthotopic switched-type transplant or a second heart in the chest. Moreover, the available donor heart likely imperfect, the issue of matching the native heart in size ceases as a factor materially increasing the pool of available replacement hearts. Whether due to an earlier surgical procedure, extracardiac repair of transposition of the great vessels, or any other earlier procedure in the chest, should a need arise to reenter, cardiac-stemal and intraperitoneal adhesions or any condition of disease that pose a hindrance to placement in the chest might prompt a direct aortocaval connection.

Also, a double heart transplant with the protective heart cage protecting the second heart positioned in the lower abdomen, for example, might be thought to benefit from adhesions, but these are likely to interfere with entry into the cage if necessary. That is tissue-to-cage adhesions might stabilize the cage but at the expense of interfering with access to the heart, and if stabilization were indeed required, then passing suture through eyelets at its surface to anchor the cage to neighboring tissue would work just as well, any initial discomfort dispelled with lidocaine and later discomfort at the anchoring points eventually blocked by fibrous encapsulation. Accordingly, unless the second heart fits snugly in its place, the heart cage should be placed with adhesion and adverse foreign body reaction preventive measures.

To minimize if not avert such an eventuality, preventive measures should include not only multiple adhesion barrier substances and membranes but periodic formative disruption using mechanical means, analogous to the balloon technique for dealing with Asherman's syndrome, or uterine synechiae (see, for example, Unanyan, A., Pivazyan, L., Kryolova, Obosyan, L., and Ishchenko, A. 2022. “Comparison of Effectiveness of Hyaluronan Gel, Intrauterine Device, and Their Combination for Preventing Adhesions in Patients after Intrauterine Surgery: Systematic Review and Meta-analysis,” Journal of Gynecology, Obstetrics, and Human Reproduction 51(4): 102334; Manchanda R. (ed.) 2020. Intra Uterine Adhesions: Diagnostic and Therapeutic Insight, New York, New York: Springer; Yang, X., Liu, Y., Li, T. C., Xia, E., Xiao, Y., and 3 others 2020. “Durations of Intrauterine Balloon Therapy and Adhesion Reformation [for 7, 14, or 28 days at 8 weeks] after Hysteroscopic Adhesiolysis: A Randomized Controlled Trial,” Reproductive BioMedicine Online 40(4): 539-546; Shi, X., Saravelos, S. H., Zhou, Q., Huang, X, Xia. E., and Li, T. C. 2019. “Prevention of Postoperative Adhesion Reformation by Intermittent Intrauterine Balloon Therapy: A Randomized Controlled Trial,” BJOG [British Journal of Obstetrics and Gynaecology] 126(10): 1259-1266).

“Morbidity and mortality are higher for cardiac reoperations than first operation due to the presence of post-operative adhesions,” (Lefort, B., El Arid, J. M., Bouquiaux, A. L., Soule, N. Chantreuil, J., and 3 others 2015. “Is Seprafilm Valuable in Infant Cardiac Redo Procedures?,” Journal of Cardiothoracic Surgery 10:47). “Given increased hospitalization, longer operative times, and longer lengh of hospital stay, post-surgical adhesions also pose a great financial burden,” (Hassanabad, A. F., Zarzycki, A. N, Jeon, K., Dundas, J. A., Vasanthan, V., Deniset. J. F., and Fedak, P. W. M. 2021. “Prevention of Post-operative Adhesions: A Comprehensive Review of Present and Emerging Strategies,” Biomolecules (Basel, Switzerland) 11(7): 1027)(see also Jin, D., Yang, S., Wu. S. Yin, M., and Kuang, H. 2022. “A Functional PVA [poly(vinyl alcohol)] Aerogel-based Membrane Obtaining Sutureability through Modified Electrospinning Technology and Achieving Promising Anti-adhesion Effect after Cardiac Surgery,” Bioactive Materials 10:355-366; Nahirniak, P. and Turna, F. 2021. “Adhesiolysis,” Online, Treasure Island, Florida: StatPearls Publishing Co.; Welle, N. J., Sajjad, H., Adkins, A., and Burns, B. 2021. “Bowel Adhesions,” Online, Treasure Island, Florida: StatPearls Publishing Co.; Iwasaki, K., Ahmadi, A. R., Qi, L., Chen, M. Wang. W., and 5 others 2019. “Pharmacological Mobilization and Recruitment of Stem Cells in Rats Stops Abdominal Adhesions after Laparotomy,” (Nature Research) Scientific Reports 9(1): 7149; Bordoni, B., Marelli, F., Morabito, B., Sacconi, B., and Severino, P. 2017. “Post-sternotomy Pain Syndrome following Cardiac Surgery: Case Report,” Journal of Pain Research 10:1163-1169. Catena, F., Di Saverio, S., Coccolini, F., Ansaloni, L., De Simone, B., Sartelli, M., and van Goor, H. 2016. “Adhesive Small Bowel Adhesions Obstruction: Evolutions in Diagnosis, Management, and Prevention,” World Journal of Gastrointestinal Surgery 8(3): 222-231; Hamid, U. I., Digney, R., Soo, L. Leung, S., and Graham, A. N. J. 2015. “Incidence and Outcome of Re-entry Injury in Redo Cardiac Surgery: Benefits of Preoperative Planning,” European Journal of Cardiothoracic Surgery 47(5): 819-823; Kang. H., Chung, Y. S., Kim, S. W., Choi, G. J., Kim, B. G., and 3 others 2015. “Effect of Temperature-sensitive Poloxamer Solution/Gel Material on Pericardial Adhesion Prevention: Supine Rabbit Model Study Mimicking Cardiac Surgery,” Online, PLoS [Public Library of Science] One 10(11): e0143359; Orhan, A., Görmüş, N., Toy, H., Görmüş, I. S. çağlayan, O., and Tanyeli, O. 2014. “Prevention of Retrosternal Pericardial Adhesions after Cardiac Surgery with Mitomycin C,” Heart, Lung, and Circulation 23(4): 357-362; Cannata, A., Petrella, D., Russo, C. F., Bruschi, G., Fratto, P., Gambacorta, M., and Martinelli, L. 2013. “Postsurgical Intrapericardial Adhesions: Mechanisms of Formation and Prevention,” Annals of Thoracic Surgery 95(5): 1818-1826; Ten Broek, R. P., Strik, C., Issa, Y., Bleichrodt. R. P., and van Goor, H. 2013. “Adhesiolysis-related Morbidity in Abdominal Surgery,” Annals of Surgery (Philadelphia, Pennsylvania) 258(1): 98-106; Kaneko, Y., Hirata, Y., Achiwa, I., Morishita, H., Soto, H., and Kobayahsi, J. 2012. “Adhesion Barrier Reduces Postoperative Adhesions after Cardiac Surgery,” Asian Cardiovascular and Thoracic Annals 20(3): 257-62; Yamauchi, T., Miyamoto, Y., Ichikawa, H., Takano, H., and Sawa, Y. 2006. “Large Vessel-Stemum Adhesion after Cardiac Surgery; a Risk-factor Analysis,” Surgery Today 36(7): 596-601; Nkere. U. U., Whawell, S. A., Sarraf. C. E., Schofeld, J. B. Thompson, J. N., and Tahlor, K. M. 1995. “Pericardial Trauma and Adhesions in Relation to Reoperative Cardiac Surgery,” Thoracic and Cardiovascular Surgery 43(6): 338-346).

Adhesions between native tissues as might be encountered during any reoperative procedure and the fibrous encapsulation or enwrapment of system components when adverse are counteracted conventionally by coating the outer surface of these with adhesion barriers or preventives such as catechol-functionalized oxime hydrogel, gelatin/polycaprolactone electrospun nanofibrous membranes; liquid paraffin, fouling-resistant zwitterionic polymers; woven fabric of modified cellulose which swells, then gels (Interceede Johnson and Johnson, New Brunswick, New Jersey; sodium hyaluronate-based bioresorbable 0.4 percent hyaluronic acid (Seprafilme, Sepracoat®; Genzyme Corporation, Cambridge, Massachusetts); dextrin hydrogel, N,O-carboxymethyl chitosan, a 4 percent solution of icodextrin (Adept®, Baxter International, Deerfield, Illinois); mitomycin C, and lactated Ringer's solution, used to wet cut tissue surfaces in order to prevent adhesions.

These and many other substances are available for the purpose (see, for example, Torres-de la Roche, L. A., Berard. V., de Wilde, M. S., Devassy. R., Wallwiener, M. and de Wilde, R. L. 2022. “Chemically Modified Hyaluronic Acid for Prevention of Post-surgical Adhesions: New Aspects of Gel Barriers Physical Profiles,” Online, MDPI [Multidisciplinary Digital Publishing Institute] Journal of Clinical Medicine (Basel, Switzerland) 11(4): 931; Fujita, M., Policastro, G. M., Burdick, A., Lam, H. T., Ungerleider, J. L., and 6 others 2021. “Preventing Post-surgical Cardiac Adhesions with a Catechol-functionalized Oxime Hydrogel,” Nature Communications 12:3764; Kikusaki S. Takagi K. Shojima T. Saku K. Fukuda T. and 5 others 2021. “Prevention of Postoperative Intrapericardial Adhesion by Dextrin Hydrogel,” General Thoracic and Cardiovascular Surgery (Tokyo, Japan) 69(9): 1326-1334; Wang, X., Xiang L., Peng. Y., Dai, Z. Hu, Y., and 4 others 2021. “Gelatin/Polycaprolactone Electrospun Nanofibrous Membranes: The Effect of Composition and Physicochemical Properties on Postoperative Cardiac Adhesion,” Online, Frontiers in Bioengineering and Biotechnology 9:792893 with correction 2022 10:862276; Park, H., Baek, S., Kang. H., and Lee, D. 2020. “Biomaterials to Prevent Post-operative Adhesion,” Materials 13(14): 3056; Zhang, E., Song, B., Shi, Y., Zhu, H., Han, X., and 3 others 2020. “Fouling-resistant Zwitterionic Polymers for Complete Prevention of Postoperative Adhesion,” Online, Proceedings of the National Academy of Sciences of the United States of America 117(50): 32046-32055; Capella-Monsonis, H., Keams, S., and Kelly, J. 2019. “Battling Adhesions: From Understanding to Prevention,” Online, BioMed Central Biomedical Engineering 1:5; Feng, B., Wang, S., Hu, D. Fu. W. Wu. J. and 4 others 2019. “Bioresorbable Electrospun Gelatin/Polycaprolactone Nanofibrous Membrane as a Barrier to Prevent Cardiac Postoperative Adhesion,” Acta Biomaterialia 83:211-220; Hanawa, T. 2019. “Titanium-Tissue Interface Reaction and Its Control with Surface Treatment,” Online, Frontiers in Bioengineering and Biotechnology 7:170; Huang, C. and Ding, D. C. 2019. “Outcomes of Adhesion Barriers in Gynecologic Surgeries: A Retrospective Study at a Medical Center,” Medicine (Baltimore, Maryland) 98: 50 (e18391; Kataria, H. and Singh, V. P. 2017. “Liquid Paraffin vs Hyaluronic Acid in Preventing Intraperitoneal Adhesions,” Indian Journal of Surgery 79(6): 539-543; Orhan, A., Gormuş, N., Toy, H., Gormuş. I. S. çağlayan, O., and Tanyeli, O. 2014, Op cit., preceding paragraph; Arung, W., Meurisse, M., and Detry, O. 2011. “Pathophysiology and Prevention of Postoperative Peritoneal Adhesions,” World Journal of Gastroenterology 17(41): 4545-4553; Diamond, M. P., Burns, E. L., Accomando, B., Mian, S., and Holmdahl, L. 2012, “Seprafilm® Adhesion Barrier: (1) A Review of Preclinical, Animal, and Human Investigational Studies,” Gynecological Surgery 8(3): 237-245; Pismensky, S. V., Kalzhanoc, Z. R., Eliseeva, M. Y., Kosmas, I. P and Mynbaev, O. A. 2011. “Severe Inflammatory Reaction Induced by Peritoneal Trauma is the Key Driving Mechanism of Postoperative Adhesion Formation,” Online, BioMed Central Surgery 11:30; Kamel, R. M. 2010. “Prevention of Postoperative Peritoneal Adhesions,” European Journal of Obstetrics and Gynecology and Reproductive Biology 50(2): 111-118; Brown, C. B., Luciano, A. A., Martin, D., Peers, E., Scrimgeour, A., and diZerega, G. S. 2007. “Adept (Icodextrin 4% Solution) Reduces Adhesions after Laparoscopic Surgery for Adhesiolysis: A Double-blind, Randomized, Controlled Study,” Fertility and Sterility 88(5): 1413-1426).

Along with surgical trauma, foreign bodies to include suture, radiation, inflammation, and hypoxia if not ischemia, infection stands as another frequent cause for the formation of postoperative adhesions, making the appropriate use of anti-inflammatoires and antimicrobials a simple and straightforward if insufficient means for reducing the postoperative formation of adhesions. “Trauma, foreign bodies, ischemia, and infections are the major factors associated with the formation of postoperative adhesions,” (Zong, X., Li, S., Chen, E., Garlick, B., Kim, K. and 6 others 2004, Op cit. above in this section)(see also Hassanabad, A. F., Zarzycki, A. N., Jeon, K., Deniset, J. F., and Fedak, P. W. M. 2021. “Post-operative Adhesions: A Comprehensive Review of Mechanisms,” Online, Biomedicines (Basel, Switzerland) 9(8): 867; Artung, W., Meurisse, M., and Detry, O. 2011. “Pathophysiology and Prevention of Postoperative Peritoneal Adhesions,” World Journal of Gastroenterology 17(41): 4545-4553; Tzianabos, A. O., Holsti, M. A., Zheng, X.-X., Stucchi, A. F., Kuchroo, V. K., and 3 others 2008. “Functional Th1 [T helper] Cells are Required for Surgical Adhesion Formation in a Murine Model,” Journal of Immunology (Baltimore, Maryland) 180(10): 6970-6976; Bothin, C., Midvedt, T. and Perbeck, L. 2003. “Orally Delivered Antibiotics Which Lower Bacterial Numbers Decrease Experimental Intra-abdominal Adhesions,” Langenbeck's Archives of Surgery 388(2): 112-115; Chung, D. R., Chitnis, T., Panzo, R. J., Kasper, D. L., Syegh, M. H., and Tzianabos, A. O. 2002. “CD4+ T Cells Regulate Surgical and Postinfectious Adhesion Formation,” Journal of Experimental Medicine 195(11): 1471-1478; Oncel, M. Kurt, N., Remzi, F. H., Gezen, C. F., Cincin, T. G., and Olcay, E. 2001. “The Effectiveness of Systemic Antibiotics in Preventing Postoperative, Intraabdominal Adhesions in an Animal Model,” Journal of Surgical Research 101(1): 52-56).

The formation of adhesions is best suppressed using the counteractive measures delineated. The nonsurgical and nonmedicinal treatment of established adhesions is limited to those in manipulable positions (see, for example, Rice, A. D., Patterson, K., Reed, E. D., Wurn, B. F., Robles, K., and 5 others 2018. “Decreasing Recurrent Bowel Obstructions, Improving Quality of Life with Physiotherapy: Controlled Study,” World Journal of Gastroenterology 24(19): 2108-2119; Bove, G. M., Chapelle, S. L., Hanion, K. E., Diamond, M. P., and Mokler, D. J. 2017. “Attenuation of Postoperative Adhesions Using a Modeled Manual Therapy,” Online, Public Library of Science One 12(6): e0178407).

As established experimentally by operating on gut-sterilized rats, exceptionally positioning a second heart in the infrarenal abdomen poses the risk of infection by the gastrointestinal microflora, promoting the formation of adhesions (see, for example, Bothin, C. G., Okada, M., Midtvedt, T., and Perbeck, L. 2001, Op cit., above in section 2a; Bothin, C. G., Okada, M., and Midtvedt, T. 1999, Op cit., above in section 2a; Bothin, C. G., and Midtvedt, T. 1992. “The Role of the Gastrointestinal Micro Flora in Post Surgical Adhesion Formation—A Study in Germfree Rats,” European Surgical Research 24(5): 309-312).

The untargeted systemic dissemination of many drugs both exposes nontargeted tissue to these drugs causing serious side effects and limits the dose, to that systemically tolerable rather than that optimal for treating the problem itself or the original, root-site of disease. This consideration dominates the treatment of a malignant tumor, for example. Intended to treat chronic or serious disease, such a system is devised to remain in place without the need for reentry or as long as an extended acute condition such as a malignancy makes necessary. Components of the system other than terminal, that is, toward and at the nidi, readily redirected to the treatment of another condition, implant removal, or explantation after successful use, especially in an elderly patient with comorbidities, is discouraged. Devised for permanent implantation, discontinued use will usually not call for removal and avert the need for reimplantation should another such condition appear.

### Use of Radiation

The application of radiation is usually intended not to supplant but rather to precede or succeed surgical excision as adjuvant where possible. Auger therapy, for example, is addressed at many points in the background section of U.S. Pat. No. 11,013,858, entitled Nonjacketing Side-entry Connectors and Prosthetic Disorder Response Systems, and addressed itself in the section of like title beginning at column 136, line 60 and continuing through column 139.

Described below in the subsection below entitled Focused Drip Brachyradiochemotherapy and in FIG. 14C is a means for expanding the applicability of Auger therapy by closed skin implantation and the high precision provided by a tiny ‘inchworm’ piezoelectric motor mounted on a nonjacketing side-entry connector as depicted in U.S. Pat. No. 11,013,858, FIGS. 13A thru 15 to advance an electron-emitting probe having the conformation depicted toward the target in minute increments with the aim of the probe stabilized regardless of the momentary posture of the patient as it is advanced by the motor (see, for example, Ku, A., Facca, J., Cai, Z., and Reilly, R. M. 2019. “Auger Electrons for Cancer Therapy-A Review,” European Journal of Nuclear Medicine and Molecular Imaging Radiopharmacy and Chemistry 4:27). Spanner, K. and Koc, B. 2016. “Piezoelectric Motors, an Overview,” Actuators 5(1): 6).

As with other components controlled by a prosthetic disorder response system, implant removal, or explantation, is usually avoided both to retain the small device or its accessory channels for subsequent use as well as to eliminate the need for reentry. Like brachytherapy, the radiation shielding of drug delivery surface ports, reservoirs, lines and their target jackets and connectors joining each to native tissue allows the local, topical, rather than systemic use of fluid radioactive agents, to include chemotherapeutics, antiangiogenics, steroids, and radioactive agents in fluid form of which many remain to be developed.

In distinct contrast to conventional brachytherapy, with a directly pipeline-targeted and tightly focused radioactive drip as part of the implanted system, nontargeted tissue is:
1. Less subject to exposure and serious side effects;
2. Each treatment does not end with the need to remove the source of radiation so that the next treatment requires reentry; and
3. Once the target tumor has been destroyed, the delivery line can remain in place to deliver different medication or can be shifted in position to focus radiation or different medication on another target endoscopically.

Magnetized impasse-jackets, shielded and unshielded, described in U.S. Pat. No. 11,389,171, entitled Integrated System for the Infixion and Retrieval of Implants, can be provided with accessory channels to dissolve superparamagnetic carrier-bound analytes, thus materially extending their capacity. Delivery segregated in space and time thus both allows discretionary drug targeting without exposure to nontargeted tissue and prevents the mixing of drugs where such would dilute and/or chemically detract from the potency of either.

That when desired each reservoir can store a mixture or ‘cocktail’ of drugs is considered obvious. Independent prescription-program driven control responsive to sensor data by the automatic disorder response system implant microcontroller or microprocessor of the drug-releasing outlet pump or drip stopcock of each drug reservoir allows immediate increasing, reducing, or stopping of each drug at the pump or drip stopcock outlet. However, adjustment at the pump or drip stopcock does not allow recovery of the drug or any portion thereof once it has been released into the drug delivery line. To reverse the pump or drip stopcock would still leave a coating of the drug along the surface of the drugline lumen.

Reservoirs are usually used for a single drug; however, if needed for another drug, the reservoir can be flushed out with a double lumen syringe that allows water to be circulated through it. Blood 92 percent water, a coating along the inner walls of the delivery line which represents a negligible dose can be rinsed forward into a target vessel with water. The reservoir can then be refilled with another drug as usual, by insertion of a hypodermic syringe through the reservoir opening in the subdermal port. Once delivered into the blood supply of the target organ or tissue, reduction or the prevention of its delivery to the target organ or tissue requires the use of 2-way, or bidirectional, diversion servovalves, which draw off flow and divert it into another vessel. Bidirectional servovalves are described below in section 6, entitled Description of the Preferred Embodiments of the Invention.

The requirements imposed by lifelong implantation in minimizing if not eliminating adverse tissue reactions, sharp corners or edges, and avoidable mass and size, along with the medical requirements then drive the design of the components. Other requirements imposed for lifelong implantation include the ability to deliver antifouling agents to counteract the accumulation if not formation of clot and biofilm in vessels, crystal in the urinary tract, and if necessary, the ability to handle radioactive substances. The timely automatic direct targeting of drugs in an ambulatory patient according to the program and/or responsive to data fed from implanted sensors to the implanted microcontroller in monomorbidity or microprocessor in multimorbidity eliminates delay in treatment and dependency upon the patient to adhere to the prescription regimen.

The placement of such a system responds to the conviction that chronic and especially progressive disease warrant decisive treatment upon initial diagnosis, not ‘half-way’ measures likely to lead to more extensive surgical or radiological treatment anyway. The application of more decisive measures capable of providing a decisive relieving of symptoms ab initio is to be preferred. Provided the benefits to be gained from the small incisions and endoscopic manipulations required to implant an automatic prosthetic disorder response system stand to materially improve the quality of the patient's life without more radical treatment, the outcome will have been worth the relatively brief invasive procedure it took to provide.

The applications for such a system range from the relative simplicity of a central line to treat a chronic condition which once placed can remain permanently, to a permanent line for the direct release into the hepatic portal vein of insulin responsive to the data supplied by glucose sensors, to bilateral lines for the direct targeting through the internal carotids of antipsychotics, anticonvulsives, antidementives, and antidepressants, for example, to the brain for release on a scheduled basis, to multiple lines to treat comorbid disease as channels in a hierarchical control system under the coordinated control of a master control microprocessor. Variation in the dose fed to either side of the brain may yield therapeutic information for use in patients and neurophysiological information through translational experimentation.

Vascular jackets and servovalves fall into four types or families, of which the different embodiments serve different medical functions. To permit a reduction in the cost of manufacture, the embodiments within each type are based upon a common platform made in different sizes. The types are 1. Diversion jackets without an electrically powered driver for continuously variable direct manual control along the lower urinary tract; 2. Highly damped nonsparking plunger solenoid diversion jackets which allow either of two states, or bistable open/closed to either outlet path, for application along the lower urinary tract and to implement a sudden compound vascular bypass, or sudden switched-organ transplantation; 3. Servomotor-driven valves used to implement metered compound vascular bypass, or sudden switched-organ transplantation; and 4. Servomotor-driven valves with direct manual adjustability to allow bypass of the carotid bifurcation during a carotid endarterectomy.

The term ductus denotes a tubular anatomical structure, regardless of type. Ductus side-entry jackets and the procedure for placing these are described in U.S. Pat. No. 11,759,186, entitled Ductus Side-entry Jackets and Prosthetic Disorder Response Systems. As shown in U.S. Pat. No. 11,013,858, entitled Nonjacketing Side-entry Connectors and Prosthetic Disorder Response Systems, the targeted release of medication into a cavity, such as the urinary bladder, or into tissue, under the control of a fully implanted automatic disorder response system executing a prescription-program is through nonjacketing side-entry connectors, while ductus side-entry jackets, are used to communicate with the lumina of tubular anatomical structures.

The term ‘body surface port’ is used to denote positioning of a port on or just beneath the skin. As indicated, most uses of the term ‘infusion’ herein denote arterial rather than venous delivery. As is standard, here ‘injection’ denotes delivery under force through a syringe and hypodermic needle or a hypotube; while ‘infusion’ is the passive delivery by gravity of a drip into a vein. Here the use of a drip is novel in that delivery is pipeline-targeted for direct topical application rather than dispersed throughout the circulation.

When the agent is radioactive and the material of the delivery line-usually an ordinary catheter-does not afford adequate shielding, the components through which the agent moves are shielded. Temporary and permanent shielding are described and illustrated in U.S. Pat. No. 11,013,858, entitled Nonjacketing Side-entry Connectors and Prosthetic Disorder Response Systems, and U.S. Pat. No. 11,759,186, entitled Ductus Side-entry Jackets and Prosthetic Disorder Response Systems, as well as shown in FIG. 14B.

Actually, prodrugs potentiated only following conversion by enzymes in the liver, many if not most drugs used in this direct or topical manner necessitates that this conversion be accomplished prior to release. That is, direct delivery thus deviates from the conventional oral, intramuscular, and intravenous routes of administration in bypassing the liver, and in so doing, necessitates exogenous conversion into the potentiated, or functional efficacious chemical structure, prior to use.

The direct pipeline-targeting of drugs by release into the supply artery at a level proximate to the target organ or tissue represents a novel route of administration, which unlike those oral, intramuscular, or intravenous, imposes two fundamental requirements on any drug administered thus: the drug must have been prepared in fluid form, and if necessary, exogenously preconverted into its potentiated form, meaning that when administered, the drug must have been preprocessed by subjection to the same enzymatic action as when used in its ordinary form where conversion into the potentiated state is entrusted to metabolic processing.

In fact, side-entry jackets, valves, and nonjacketing side-entry connectors allow the direct delivery of drugs in two ways: by injection into a port at the body surface such as those shown in FIGS. 26A and 26C, and by the transluminal passage of a fine endoscope with syringe through the port and into the jacket or connector. When injected at the port, the bolus if not free flowing is backed up by a column of water. The passage of a fine cabled devices into the jacket or connector includes fiberscopes, for example, for diagnosis and excimer lasers, for example, for therapy. The effectiveness of delivery thus is exemplified in the administration of antibiotics, for example. Implantation of a line to target antibiotics is justified only when reinfection is frequent and ongoing; otherwise, such use pertains to permanent jackets and connectors placed to treat a chronic primary disorder or graft organ where infection arises as an intercurrent problem.

When administered immediately upon the detection of infection, the confrontation of the pathogen with an antimicrobial truncates if not preempts the ability of the pathogen to deploy its defenses such as release an exotoxin in a sufficient amount to induce an active infection, form an adherent biofilm, and secrete enzymes such as protease, hyaluronidase, neuraminidase, elastase, and collagenase to continue its penetration through tissue (Tunkel, A. R. 2006. “Biology of Infectious Disease,” Chapter 167, page 1388, in The Merck Manual, Whitehouse Station, New Jersey: Merck Research Laboratories) that facilitate penetration through and the gaining of a foothold in neighboring tissue.

With an automatic totally implanted prosthetic disorder response system, the detection and response to infection is automated so that the patient may not even be aware that an infection or a circulatory crisis, for example, had occurred before it had been eradicated. To accomplish this requires sensors able to detect analytes associated with pathogens such as exotoxins and enzymes such as proteases associated with infection and a prescription-program that prescribes the automatic response, which in most instances, will consist of releasing a counteractive drug or drugs.

As addressed below in subsection f. below, entitled Inflammation, biosensors only usable in the clinic are those least desirable, sensors which do not incorporate biological material, especially when subject to degeneration or depletion, are much to be preferred, as these are consistent with a fully implanted or closed-skin application not requiring periodic servicing much less surgical reentry to replenish the biological substance. If necessary, sensors which incorporate biological analyte reactants, or biosensors, such as the substrates of specific enzymes, can be routinely replenished or even replaced through service or accessory channels; however, it is preferable that until a replacement sensor not requiring periodic servicing becomes available, those requiring periodic maintenance be provided with access passageways.

Eventually, sensors will be not only be “free-standing” but incorporated as integral in side-entry jackets, vascular valves, and nonjacketing side-entry connectors, eliminating any need for later access. Until the latter become available, for the purpose of monitoring more serious conditions, biosensors which do require periodic servicing may have to serve in place of permanently implantable sensors. Based upon the appearance, medical history, and genetic diagnosis of the patient, a current status of metabolic syndrome or some other condition capable of inducing a crisis situation recommends the incorporation of sensors to detect this condition at any time.

An ideal sensor for this purpose is one which can be implanted indefinitely for the purpose of ‘lying in wait’ to signal the appearance of a premonitory prodromal analyte indicative of an impending crisis which evidence such as that mentioned had led the diagnostician to suspect. When the sensor has been supported by the placement of a drug reservoir or reservoirs containing medication to counteract the progress of the emerging incident, signaling the diagnostic analyte to the control system can initiate treatment before the crisis has an opportunity to develop.

Should sensors that once implanted never require periodic maintenance or replacement remain unavailable, the better of two alternatives are sensors that are serviceable for use away from the clinic, the alternative-those only usable in the clinic, least advantageous (see, for example, Mobasheri, A. 2021. “Biosensors for the Multiplex Detection of Inflammatory Disease Biomarkers,” Biosensors (Basel, Switzerland) 11(1): 11; Koydemir, H. C. and Ozcan, A. 2018. “Wearable and Implantable Sensors for Biomedical Applications,” Annual Review of Analytical Chemistry (Palo Alto, California) 11(1): 127-146; Salvo, P., Dini, V., Kirchhain, A., Janowska, A., Oranges, T., and 4 others 2017, “Sensors and Biosensors for C-Reactive Protein, Temperature, and pH, and Their Applications for Monitoring Wound Healing: A Review,” Sensors (Basel, Switzerland). 17(12): 2952; Bhalla, N. Jolly, P., Formisano, N., and Estrela, P. 2016. “Introduction to Biosensors,” Essays in Biochemistry 60(1): 1-8; Justino, C. I., Freitas, A. C., Amaral, J. P., Rocha-Santos, T. A., Cardoso, S., and Duarte, A. C. 2013. “Disposable Immunosensors for C-reactive Protein Based on Carbon Nanotubes Field Effect Transistors,” Talanta 108:165-170; Sohn, Y.-S. and Kim, Y. T. 2008. “Field-effect-transistor Type C-reactive Protein Sensor Using Cysteine-tagged Protein G,” Electronics Letters 44(16): 955-956; Klueh, U., Kaur, M., Montrose, D. C., and Kreutzer, D. L. 2007, Op cit. above in section 2a, entitled Background of the Invention. General; Pearson, T. A., Mensah, G. A., Alexander R. W., Anderson J. L., Cannon, R. O. III, Criqui, M., Fadl Y. Y., and 8 others 2003. “Markers of Inflammation and Cardiovascular Disease,” Circulation 107(3): 499-511).

Intracorporeal biosensors which can be used in the clinic can be delivered to the site to be diagnosed by insertion through a drugline or service channel of the side-entry jacket, valve, or nonjacketing side-entry connector previously placed to deliver medication to the site of disease if not for this purpose as well. In contrast, implantable electronic sensors are not confined to use in the clinic but can signal the onset of prodromal indications of imminent disease while the patient remains occupied and unaware of the condition, can communicate with the clinic by wireless means, and incorporate no degradable organic material, making this type of sensor more consistent with the objectives sought in the use of a fully implanted prosthetic disorder response system.

Not referred to here are means of the innate immune system for the detection of inflammation, such as cyclic GMP [guanine monophosphate]/AMP [adenine monophosphate], or cGAS, stimulator of interferon gene [STING] inflammasome activating and Toll-like receptor pathways for the self-sensing and induction of cytosolic nucleic acid, no less pertinent to the detection of inflammation associated with microbial infection and tumorigenesis and responsible for the induction of pyroptosis and the secretion of mature cytokines prominent in the integrated stress response due to the trauma inflicted by conventional solid organ transplantation, for example (see, for example, Watson, R. O., Bell, S. L., MacDuff, D. A., Kimmey, J. M., Diner, E. J., and 5 others 2015. “The Cytosolic Sensor cGAS Detects Mycobacterium tuberculosis DNA to Induce Type 1 Interferons and Activate Autophagy,” Cell Host and Microbe 17(6): 811-819; Martinon, F., Burns, K., and Tschopp, J. 2002. “The Inflammasome: A Molecular Platform Triggering Activation of Inflammatory Caspases and Processing of Pro IL [interleukin]-beta,” Molecular Cell 10(2): 417-426).

Another option for the continuous monitoring of biosignals are sensors which are wearable. These have the advantages of providing constant surveillance without implantation within the body and therefore ready access should servicing or replacement become necessary (see, for example, Mukhopadhyay, S. C., Suryadevara, N. K., and Nag, A. 2021. “Wearable Sensors and Systems in the IoT [internet of things],” Sensors (Basel, Switzerland) 21(23): 7880; Perez, A. J. and Zeadally, S. 2021. “Recent Advances in Wearable Sensing Technologies,” Sensors (Basel, Switzerland) 21(20): 6828; Stuart, T., Cai, L., Burton, A., and Gutruf, P. 2021. “Wireless and Battery-free Platforms for Collection of Biosignals,” Biosensors and Bioelectronics 178:113007; Rodrigues, D., Barbosa, A. I., Rebelo, R., Kwon, I. K., Reis, R. L., and Correlo, V. M. 2020. “Skin-integrated Wearable Systems and Implantable Biosensors: A Comprehensive Review,” Biosensors (Basel, Switzerland) 10(7): 79; Gao, W., Ota, H., Kiriya, D., Takei, K., and Javey, A. 2019. “Flexible Electronics toward Wearable Sensing,” (American Chemical Society) Accounts of Chemical Research 52(3): 523-533; Park, Y. G., Lee, S., and Park, J. U. 2019. “Recent Progress in Wireless Sensors for Wearable Electronics,” Sensors (Basel, Switzerland) 19(20): 4353; Heikeneld, A., Jajack, A., Rogers, J., Gutruf, L, Tian, T., and 6 others 2018. “Wearable Sensors: Modalities, Challenges, and Prospects,” Lab on a Chip 18(2): 217-248; Islam, T., and Mukhophadayay, S. B. 2017. Wearable Sensors, Bristol, England: IoP [Institute of Physics] Publishing; Patel, S., Park, H., Bonato, P., Chan, L., and Rodgers, M. 2012. “A Review of Wearable Sensors and Systems with Application in Rehabilitation,” Journal of NeuroEngineering and Rehabilitation/Pubmed Central 9:21).

Biosensensors which can be periodically replaced or have their biological material replaced through a mainline or sideline (drugline, service channel, accessory channel) are intermediate in value between those usually only in the clinic and sensors that can be implanted without periodic revisiting. This is because a primary object of an implanted response system is precisely to interdict the unfolding of an exigent condition upon its appearance and prevent its progress before it does further injury. The onset of a myocardial infarction, for example, would seldom arise when the patient happened to be at the clinic.

The term ‘immunosensors’ is applicable to all sensors used to detect antigens whether pathogens, allergens, tumors, implants as foreign bodies, and graft organs as provoking an immune reaction. From this standpoint, immunosensors can usually be classified under any of the categories shown below. The seeding of prospective graft organs with rejection-signaling biosensor cells prior to transplantation by Chin et al. 2019, cited below under section e, entitled Graft Organ Rejection Analytes and Immunosensors, is especially noteworthy even though it differs from the methods set forth herein as performed following harvesting and placement on an ex vivo perfusion machine of the graft organ, which distinction is, however, readily overcome.

Side-entry jackets, servovalves, connectors, druglines, and sensors are specifically designed to eliminate leaks, perforations, and minimize adverse, or foreign body tissue reaction (see, for example, Dong, J., Wang, W., Zhou, W., Zhang, S., Li, M., and 5 others 2022. “Immunomodulatory Biomaterials for Implant-associated Infections: From Conventional to Advanced Therapeutic Strategies,” Online, BioMed Central Biomaterials Research 26(1): 72; Thorson, T. J., Gurlin, R. E., Botvinick, E. L., and Mohraz, A. 2019. “Bijel-templated Implantable Biomaterials for Enhancing Tissue Integration and Vascularization,” Online, Acta Biomaterialia 94:173-182; de la Oliva, N., Navarro, X., and Del Valle, J. 2018. “Time Course Study of Long-term Biocompatibility and Foreign Body Reaction to Intraneural Polymide-based Implant,” Journal of Biomedical Materials Research Part A 106(3): 746-757; Gray, M. E., Meehan, J., Blair, E. O., Ward, C., Langdon, S. P., and 6 others 2018. “Biocompatibility of Common Implantable Sensor Materials in a Tumor Xenograft Model,” Online, Journal of Biomedical Materials Research. Part B. Applied Biomaterials 107(5): 1620-1633; Klopfleisch, R. and Jung, F. 2017. “The Pathology of the Foreign Body Reaction against Biomaterials,” Journal of Biomedical Materials Research Part A 105(3): 927-940; Wang, Y., Vaddiraju, S., Gu, B., Papadimitrakopoulos, F., and Burgess, D. J. 2015. “Foreign Body Reaction to Implantable Biosensors: Effects of Tissue Trauma and Implant Size,” Online, Journal of Diabetes Science and Technology 9(5): 966-977; Avula, M. N., Rao, A. N., McGill, L. D., Grainger, D. W., and Solzbacher, F. 2013. “Modulation of the Foreign Body Response to Implanted Sensor Models through Device-based Delivery of the Tyrosine Kinase Inhibitor, Mastinib,” Biomaterials 34(38): 9737-9746; Kim, S.-J., Lee, D.-S., Kim, I.-G., Sohn, D.-W., Park, J.-Y., Choi, B.-K., and Kim, S.-W. 2012. “Evaluation of the Biocompatibility of a Coating Material for an Implantable Bladder Volume Sensor,” Online, Kaohsiung Journal of Medical Sciences 28(3): 123-129; van Putten, S. M., Wubben, M., Plantinga, J. A., Hennink, W. E., van Luyn, M. J., and Harmsen, M. C. 2011. “Endotoxin Contamination Delays the Foreign Body Reaction,” Journal of Biomedical Materials Research Part A 98(4): 527-534; Higgins, D. M., Basaraba, R. J., Hohnbaum, A. C., Lee, E. J. Grainger, D. W., and Gonzalez-Juarrero, M. 2009. “Localized Immunosuppressive Environment in the Foreign Body Response to Implanted Biomaterials,” Online, American Journal of Pathology 175(1): 161-170; Anderson, J. M., Rodriguez, A., ad Chang, D. T. 2008. “Foreign Body Reaction to Biomaterials,” Online, Seminars in Immunology 20(2): 86-100).

The intrusion into the body of an alien object is always rejected to some extent, so that the idea that a noble metal such as gold or a biocompatible plastic such as polyether ether ketone will completely elude an adverse tissue reaction is mistaken-rather, when the intensity of the reaction can be kept to a minimum, a coating of a topical anesthetic will dispel discomfort following placement, and the envelopment of an isolating and protective fibrous capsule by the immune system will eliminate long-term irritation.

For technology in development, the ability for a sensor, wearable sensor, point of care, or lab-on-a-chip to identify an analyte based upon a biopsy sample is seldom unadaptable for intracorporeal application as a tiny implant. In that implanted sensors inherently report their location as well as their analyte, these are critically superior to laboratory testing such as for c-reactive protein which provides no indication as to the location within the body of the inflammation.

Sensors referenced are those implantable or eventually adaptable for implanted use, so that otherwise valuable insights, such as offered by Swaminathan, V., Mythreye, K., O'Brien, E. T., Berchuck, A., and Blobe, G. C., and Superfine, R. 2011. “Mechanical Stiffness Grades Metastatic Potential in Patient Tumor Cells and in Cancer Cell Lines,” Cancer Research 71(15): 5075-5080 have been omitted as unamenable to implantation. To avoid a profusion of categories, many for one or a very few entries, any such breakdown of categories in a field of medical research must include some references that only loosely fit into one of the major categories.

Apart from use in a prosthetic disorder response system, the applications of vascular valves and servovalves are few, mostly for use in a urinary diversion system such as those described below in section 2c, entitled Ductus Side-entry Jackets and Vascular Valves in Urinary Diversion and shown in FIGS. 28 thru 30. As components in a prosthetic disorder response system, servovalves are an integral part of the system such that these could be properly considered apart from this context no more than might any other components, such as sensors.

Implanted or worn, components in a prosthetic disorder response system such as sensors make it possible for the system to provide an informed and measured response to the instant condition of the patient. From a medical standpoint, this diagnostic and therapeutic immediacy is critically superior to the delay associated with diagnostic testing in a laboratory followed by the additional delay incurred before being seen in a clinic. Delay is the cardinal cause for an unsatisfactory outcome in the treatment of stroke, for example (see, for example, di Biase, L, Bonura, A., Caminiti, M. L., Pecoraro, P. M., and Di Lazzaro, V. 2022. “Neurophysiology Tools to Lower the Stroke Onset to Treatment Time during the Golden Hour: Microwaves, Bioelectrical Impedance and Near Infrared Spectroscopy,” Online, Annals of Medicine 54(1): 2658-2671; Chowdhury, S. Z., Baskar, P. S., and Bhaskar, S. 2021. “Effect of Prehospital Workflow Optimization on Treatment Delays and Clinical Outcomes in Acute Ischemic Stroke: A Systematic Review and Meta-analysis,” Online, Academic Emergency Medicine 28(7): 781-801; Huang, Q., Zhang, J. Z., Xu, W. D., and Wu, J. 2018. “Generalization of the Right Acute Stroke Promotive Strategies in Reducing Delays of Intravenous Thrombolysis for Acute Ischemic Stroke: A Meta-analysis,” Online, Medicine (Baltimore, Maryland) 97(25): e11205).

This unprecedented advancement will prompt the development of new sensors and other system components for which the utility could not have been predicted. With an implanted prosthetic disorder response system effectuated, the opportunity and desirability of developing implantable or wearable sensors to provide information not immediately available previously will be obvious. Many sensors that could be modified for implantation have not simply because absent an implantable prosthetic disorder response system, the inducement is lacking.

For example, implanted sensors to detect troponin originating in either of two hearts following a compound vascular bypass, or switched-organ, heterotopic double heart transplant to signal a reduction in the absolute stroke volume, or ejection fraction, in either heart not marked by a desynchronization as would be detected electronically could be applied to the release of an inotrope to stimulate the stronger of the two to compensate for the impairment of the other; a new capability prompts the development of new technology to take greater advantage of it.

Implantable sensors to detect specific analytes such as glucose level in diabetes, addressed below in ths section, serum uric acid in metabolic syndrome, and creatine kinase and troponin sensors to signal a cardiac emergency, for example, are already numerous (see, for example, Wang, L, Zhang, T., Liu, Y., Tang, F., and Xue, F. 2020. “Association of Serum Uric Acid with Metabolic Syndrome and Its Components: A Mendelian Randomization Analysis,” Online, BioMed Research International 2020:6238693;

Chin, L. Y., Carroll, C., Raigani, S., Detelich, D. M., Tessier, S. N., and 6 others 2019. “Ex Vivo Perfusion-based Engraftment of Genetically Engineered Cell Sensors into Transplantable Organs,” Online, PLoS [Public Library of Science] One 14(12): e0225222; Vashistha, R., Dangi, A. K., Kumar, A., Chhabra, D., and Shukla, P. 2018. “Futuristic Biosensors for Cardiac Health Care: An Artificial Intelligence Approach,” Online, 3 Biotech 8(8): 358; Virsaladzi, D. K., Tetradze, L. O., Dzhavashvilli, L. V., Esaliia, N. G., and Tananashvilli, D. E. 2007. “Levels of Uric Acid in Serum in Patients with Metabolic Syndrome,” Georgian Medical News(146): 35-37; and creatine kinase and troponin to strongly indicate if not prove a fibrillatory crisis or myocardial infarction (see, for example, Cabaniss, C. D.1990. “Creatine Kinase,” (chapter 32) in Walker, H. K., Hall, W. D., and Hurst, J. W., (eds.), Clinical Methods: The History, Physical, and Laboratory Examinations,” Boston, Massachusetts: Butterworth Publishers).

Implantable sensors may be multi-analyte detecting (see, for example, Zhang, M., Wang, X., Huang, Z, and Rao, W. 2020. “Liquid Metal Based Flexible and Implantable Biosensors,” Online, Biosensors (Basel, Switzerland) 10(11): 170). Nonspecific, an increased in sedimentation rate will indicate inflammation whether due to autoimmune disease, infection, malignancy, and/or a hematological (blood or marrow) disorder (see, for example, Tishkowski, K. and Gupta, V. 2022. “Erythrocyte Sedimentation Rate,” Online, Treasure Island, Florida: StatPearls Publishing Co.). With a known cause, it is an index as to the degree of increase or decrease in the disease process. However, an implantable sensor to measure sedimentation rate is inherently refractory to development.

One approach is to devise a sedimentation rate device which is so small that it is effectively oblivious to a lack of stability (see, for example, Hashemi, R., Majidi, A., Mohamed, H., Amini, A., Najari, F. and Tabatabaey, A. 2015. “Erythrocyte Sedimentation Rate Measurement Using as a Rapid Alternative to the Westergren Method,” Online, Archives of Academic Emergency Medicine (Tehran, Iran) 3(2): 50-53). However, as a diagnostic test, sedimentation rate is too nonspecific and elusive of reduction to practice for use in a preferably fully implanted prosthetic disorder response system.

As the analytes themselves, an integrated stress response and cytokine storm, or hypercytokinemia, are likewise nonspecific; however, exigent and more likely amenable to attribution without further testing, these are more constructively monitored with the aid of sensors, often wearable. Spectral reviews of the field in development are available (see, for example, Rodrigues, D., Barbosa, A. I., Rebelo, R., Kwon, I. K., Reis, R. L, and Correlo, V. M. 2020. “Skin-integrated Wearable Systems and Implantable Biosensors: A Comprehensive Review,” Online, Biosensors (Basel, Switzerland) 10(7): 79; Wang, N., Yang, A., Fu, Y., Li, Y., and Yan, F. 2019. “Functionalized Organic Thin Film Transistors for Biosensing,” (American Chemical Society) Accounts of Chemical Research 52(2): 277-287).

Exceptionally, sensors not requiring or refractory to permanent implantation due to the development of a fibrous capsule that obstructs its sight, for example, can be used in wearable form; however, even a wearable sensor cannot be a simple radiation exposure badge, for example, but must be capable of transmitting its data to an independent microcontroller or node in a hierarchical control system reporting to a master control microprocessor.

Such an automatic system, delineated in copending application Ser. No. 17/689,880, entitled Prosthetic Disorder Response Systems, is diagnostic and therapeutic across a number of morbidities and programmed to command the optimal therapy across the sum of morbidities in the specific patient. Extracorporeal apparatus and means requiring replenishable substances are usually embodiments which are suitable for implantation. While not directed to detection as such, the existing literature provides indications as to how this would be accomplished.

Addressed above and in other sections, optogenetics has the potential to aid in the study, detection, and selective eradication of bacteria and other pathogens (see, for example, Sheets, M. B., Tague, N., and Dunlop, M. J. 2023. “An Optogenetic Toolkit for Light-inducible Antibiotic Resistance,” Online, Nature Communications (London, England) 14(1): 1034; Chia, N., Lee, S. Y., and Tong, Y. 2022. “Optogenetic Tools for Microbial Synthetic Biology,” Biotechnology Advances 59:107953: Lindner, F. and Diepold, A. 2022. “Optogenetics in Bacteria-Applications and Opportunities,” Online, FEMS [Federation of European Microbiological Societies] Microbiology Reviews 46(2): fuab055; Ohlendorf, R. and Möglich, A. 2022. “Light-regulated Gene Expression in Bacteria: Fundamentals, Advances, and Perspectives,” Online, Frontiers in Bioengineering and Biotechnology 10:1029403; Robinson, C. M., Short, N. E., and Riglar, D. T. 2022. “Achieving Spatially Precise Diagnosis and Therapy in Mammalian Gut Using Synthetic Microbial Gene Circuits,” Online, Frontiers in Bioengineering and Biotechnology 10:959441; Baumschlager, A. and Khammash, M. 2021. “Synthetic Biological Approaches for Optogenetics and Tools for Transcriptional LightOcontrol in Bacteria,” Advanced Biology (Weinheim, Germany) 5(5): e2000256; Sheets, M. B., Wong, W. W., and Dunlop, M. J., 2020. “Light-inducible Recombinases for Bacterial Optogenetics,” Online, American Chemical Society Synthetic Biology 9(2): 227-235; Arroyo-Olarte, R. D., Thurow, L., Kozjak-Pavlovic, V. and Gupta, N. 2018. “Illuminating Pathogen-Host Intimacy through Optogenetics,” Online, PLoS [Public Library of Science Pathogens] 14(7): e1007046; Landry, B. P. and Tabor, J. J. 2017. “Engineering Diagnostic and Therapeutic Gut Bacteria,” Online, Microbiology Spectrum 5(5) 15; Guliy, O. I., Bunin, V. D., Korzhenevich, V. I., Volkov, A. A., and Ignatov, O. V. 2016. “Electrooptical Analysis of Microbial Cell Suspensions for Determination of Antibiotic Resistance” Cell Biochemistry and Biophysics 74(4): 537-544).

### Sensors

Following is information pertaining to implantable or potentially implantable sensors for a. Bacteria, b. Viruses, c. Tumor cells, d Sundry infectious pathogens, e. Graft organ rejection analytes and immunosensors, f. Inflammation, g. Low or high-density lipoprotein, h. Coagulation, thrombus, i. Stress, crisis, cytokines, j. Stroke, or cerebral infarction, k. Radiation, l. Imminent cardiac arrest, m. Urinary crystal, and n. Adverse reactions to drugs.

Artificial intelligence and sensor feedback will enter into the continuous assessment by the implanted disorder response system across the sum of therapeutic factors to achieve optimal homeostasis for the specific patient. Ingestible sensors to detect the use of illegal substances or adherence to a drug regimen are unsuitable for use in an implanted disorder response system which administers the drugs used by the system.

Sensors may or may not be implantable. When not, adaptation into an implantable or at least a wearable form for use with an otherwise fully implanted prosthetic disorder system can still represent an advancement; however, the need to periodically replace an organic, or biomimetic, component in a biosensor can make wearability combined with a means for access to the body interior with Bluetooth transmission to the master microprocessor preferable to permanent implantability (see, for example, Pohanka, M. 2022. “Diagnoses Based on C-reactive Protein Point-of-care Tests,” Online, Biosensors (Basel, Switzerland) 12(5): 344; Carnicer-Lombarte, A., Chen, S.-T., Malliaras, G. G., and Barone, D. G. 2021. Foreign Body Reaction to Implanted Biomaterials and Its Impact in Nerve Neuroprosthetics,” Online, Frontiers in Bioengineering and Biotechnology (Amsterdam, North Holland) 9:622524; Kharbikar, B. N., Chendke, G. S., and Desai, T. A. 2021. “Modulating the Foreign Body Response of Implants for Diabetes Treatment,” Advanced Drug Delivery Reviews 174:87-113; Park, K. C. and Choi, H. J. 2021. “Impaction of a Continuous Glucose Monitoring Sensor,” Archives of Plastic Surgery 48(4): 392-394; Piech, D. K., Johnson, B. C., Shen, K., Ghanbari, M. M., Li, K. Y., and 5 others 2020. “A Wireless Millimetre-scale Implantable Neural Stimulator with Ultrasonically Powered Bidirectional Communication,” Nature Biomedical Engineering 4(2): 207-222; Rodrigues, D., Barbosa, A. I., Rebelo, R., Kwon, I. K., Reis, R. L., and Correlo, V. M. 2020, Op cit. above in section 1a, entitled Background. General; Ghorbani, F., Abbaszadeh, H., Mehdizadeh, A., Ebrahimi-Warkiani, M., Rashidi M. R., and Yousefi, M. 2019. “Biosensors and Nanobiosensors for Rapid Detection of Autoimmune Diseases: A Review” Mikrochimica Acta (Vienna, Austria) 186(12): 838; Gray, M. E., Meehan, J., Blair, E. O., Ward, C., Langdon, S. P., avnd 6 others 2019. “Biocompatibility of Common Implantable Sensor Materials in a Tumor Xenograft Model,” Journal of Biomedical Materials Research. Part B. Applications of Biomaterials 107(5): 1620-1633; Zhou, J., Ma, Z., Hong, X., Wu, H. M., Ma. S. Y., and 4 others 2019. “Top-down Strategy of Implantable Biosensor Using Adaptable, Porous Hollow Fibrous Membrane,” American Chemical Society Sensors 4(4): 931-937; Gray, M. Meehan, J., Ward, C., Langdon, S. P., Kunkler, I. H., Murray, A., and Argyle, D. 2018. “Implantable Biosensors and Their Contribution to the Future of Precision Medicine,” Veterinary Journal 239:21-29; Scholten, K. and Meng. E. 2018. “A Review of Implantable Biosensors for Closed-loop Glucose Control and Other Drug Delivery Applications,” International Journal of Pharmaceutics 544(2): 319-334; Homing, M., Haulena, M., Rosenberg, J. F. and Nordstrom, C. 2017. “Intraperitoneal Implantation of Life-long Telemetry Transmitters in Three Rehabilitated Harbor Seal Pups,” Online, BioMed Central Veterinary Research 13(1): 139; Salvo, P., Dini, V., Kirchhain, A., Janowska, A., Oranges. T., and 4 others 2017. “Sensors and Biosensors for C-reactive Protein, Temperature, and pH, and Their Applications for Monitoring Wound Healing: A Review,” Online, Sensors (Basel, Switzerland) 17(12): 2952; Zhang, X., Zambrano, A., Lin, Z. T., Xing. Y., Rippy, J., and Wu, T. 2017. “Immunosensors for Biomarker Detection in Autoimmune Diseases,” Archivum Immunologiae et Therapiae Experimentalis (Warsaw, Poland) 65(2): 111-121; De Vlaminck, I., Martin, L., Kertesz, M., Patel, K., Kowarsky, M., and 11 others 2015, Op cit., preceding paragraph; Wang. Y., Vaddiraju. S., Gu. B. Papadimitrakopoulos, F., and Burgess, D. J. 2015. “Foreign Body Reaction to Implantable Biosensors: Effects of Tissue Trauma and Implant Size,” Journal of Diabetes Science and Technology 9(5): 966-977; Wang. Y., Papadimitrakopoulos, F., and Burgess, D. J. 2013. “Polymeric “Smart” Coatings to Prevent Foreign Body Response to Implantable Biosensors,” Journal of Controlled Release 169(3): 341-347; Miyazaki, M. 2002. “The Relevance of a Simultaneous Measurement of WBC and CRP as a POCT (Point of Care Testing) of the Inflammatory Markers Running Title: the POCT of WBC and CRP,” (in Japanese-English abstract at Pubmed) Rinsho Byori (Japanese Journal of Clinical Pathology) 50(11): 1029-1034).

Not all pathogens and conditions of disease call for implanted sensors; pathogenic protozoa and nematodes, for example, are readily identifiable from their observable symptoms. For those that are not, sensors are under intensive development. Sensors incorporable into side-entry jackets, vascular valves, servovalves, inline coupling jackets, bloodlines, druglines, nonjacketing side-entry connectors, tie-lines, and confluence conduits include but are not limited to:

a. Bacteria. (see, for example, Wang, P., Sun, H., Yang. W., and Fang. Y. 2022. “Optical Methods for Label-free Detection of Bacteria,” Online, Biosensors (Basel, Switzerland) 12(12): 1171; Arreguin-Campos, R., Jimenez-Monroy, K. L., Dilien, H., Cleij. T. J., van Grinsven, B., and Eersels. K. 2021. “Imprinted Polymers as Synthetic Receptors in Sensors for Food Safety,” Online, Biosensors (Basel, Switzerland) 11(2): 46; Bergwerff, A. A. and Debast. S. B. 2021. “Modernization of Control of Pathogenic Micro-organisms in the Food-chain Requires a Durable Role for Immunoaffinity-based Detection Methodology-A Review,” Online, Foods 10(4): 832; Cui, J., Zhou, M., Li, Y., Liang, Z., Li, Y., and 5 others 2021. A New Optical Fiber Probe-based Quantum Dots Immunofluorescence “Biosensors in the Detection of Staphylococcus aureus,” Online, Frontiers in Cellular and Infection Microbiology 11:665241; Idil, N., Bakhshpour, M., Perçin, I., and Mattiasson, B. 2021. “Whole Cell Recognition of Staphylococcus aureus Using Biomimetic SPR [surface plasmon resonance] Sensors,” Biosensors (Basel, Switzerland) 11(5): 140; Martinez, O. M. 2020. Op cit. above in this section; Yaghoubi, M., Rahimi, F., Negahdari, B., Rezayan, A. H., and Shafiekhani, A. 2020. “A Lectin-coupled Porous Silicon-based Biosensor: Llabel-free Optical Detection of Bacteria in a Real-time Mode,” (Nature Research) Scientific Reports 10:16017; Divagar, M., Sriramprabha, R., Sornambikai, S., Ponpandian, N., and Viswanathan C. 2019. “Surface Imprinted Ag Decorated MnO: Thin Film Electrodes for the Synergic Electrochemical Detection of Bacterial Pathogens Journal of the Electrochemical Society 166(2): G1-G9; Furst. A. L. and Francis, M. B. 2019. “Impedance-based Detection of Bacteria,” Chemical Reviews 119(1): 700-726; Jafari, H., Amiri, M., Abdi. E., Navid, S. L., Bouckaert, J., and 3 others 2019. “Entrapment of Uropathogenic E. coli Cells into Ultra-thin Sol-gel Matrices on Gold Thin Films: A Low Cost Alternative for Impedimetric Bacteria Sensing” Biosensensors and Bioelectronics 124-125:161-166: Wilson D. Materon E, Ibañez-Redin G, Faria R C. Correa D S, Oliveira O N Jr. 2019. “Erratum to. Electrical Detection of Pathogenic Bacteria in Food Samples Using Information Visualization Methods with a Sensor Based on Magnetic Nanoparticles Functionalized with Antimicrobial Peptides [Talanta 194(2019) 611-618],” Online, Talanta (Amsterdam, North Holland, Netherlands) 1:200:562.; Wilson, D., Materon, E., Ibanez-Redin, G., Faria, R. C., Correa, D. S., and Oliveira, O. N. Jr. 2019. “Electrical Detection of Pathogenic Bacteria in Food Samples Using Information Visualization Methods with a Sensor Based on Magnetic Nanoparticles Functionalized with Antimicrobial Peptides,” Online, Talanta (Amsterdam, North Holland, Netherlands) 194:611-618; Zhang, Z., Zhou, J., and Du. X. 2019. “Electrochemical Biosensors for Detection of Foodborne Pathogens,” Micromachines (Basel, Switzerland) 10(4): 1-9; Dulay, M. T., Zaman, N., Jaramillo, D., Mody, A. C., and Zare, R. N. 2018. “Pathogen-imprinted Organosiloxane Polymers as Selective Biosensors for the Detection of Targeted E. coli,” C (Journal of Carbon Research)(Basel, Switzerland) 4(2): 29; Ashley, J., Shahbazi. M.-A., Kant, K., Chidambara, V. A., Wolff, A., Bang, D. D., and Sun, Y. 2017. “Molecularly Imprinted Polymers for Sample Preparation and Biosensing in Food Analysis: Progress and Perspectives,” Biosensensors and Bioelectronics 91:606-615; Golabi, M., Kuralay, F., Jager, E. W. H., Beni, V., and Turner A. P. F. 2017. “Electrochemical Bacterial Detection Using poly(3-aminophenylboronic Acid)-based Imprinted Polymer,” Biosensensors and Bioelectronics 93:87-93; Idil, N. and Mattiasson, B. 2017. “Imprinting of Microorganisms for Biosensor Applications,” Sensors (Basel, Switzerland) 17(4), 708; Ionescu, R. E. 2017. “Biosensor Platforms for Rapid Detection of E. coli Bacteria,” in Recent Advances on Physiology. Pathogenesis and Biotechnological Applications, InTechOpen: London, England, pages 275-289; Wu, G., Meyyappan, M., and Lai, K. W. C. 2016. “Graphene Field-effect Transistors-based Biosensors for Escherichia coli Detection:” Institute of Electrical and Electronics Engineers 16th International Conference on Nanotechnology (IEEE-NANO), pages 22-25; Abbaspour, A., Norouz- Sarvestani, F., Noori, A., and Soltani, N. 2015. “Aptamer-conjugated Silver Nanoparticles for Electrochemical Dual-aptamer-based Sandwich Detection of Staphylococcus aureus,” Biosensensors and Bioelectronics 68:149-155; Barreiros dos Santos, M., Azevedo, S., Agusil, J. P., Prieto-Simon, B., Sporer, C., and 4 others 2015. “Label-free ITO [indium-tin oxide]-based Immunosensor for the Detection of Very Low Concentrations of Pathogenic Bacteria,” Bioelectrochemistry (Amsterdam, North Holland, Netherlands) 101:146-152; Wang. Y. and Alocilja, E. C. 2015. “Gold Nanoparticle-labeled Biosensor for Rapid and Sensitive Detection of Bacterial Pathogens,” Journal of Biological Engineering 9(1): 16; Zhou, Y. and Ramasamy, R. P. 2015. “Phage-based Electrochemical Biosensors for Detection of Pathogenic Bacteria,” Electrochemical Society Transactions 69(38): 1-8; Ahmed, A., Rushworth, J. V., Hirst, N. A., and Millner, P. A. 2014. “Biosensors for Whole-cell Bacterial Detection,” Clinical Microbiology Reviews 27(3): 631-646; Etayash, H., Jiang, K., Thundat. T., and Kaur, K. 2014. “Impedimetric Detection of Pathogenic Gram- positive Bacteria Using an Antimicrobial Peptide from Class IIa Bacteriocins,” Analytical Chemistry 86(3): 1693-1700; Qi. P., Wan, Y., and Zhang, D. 2013. “Impedimetric Biosensor Based on Cell- mediated Bioimprinted Films for Bacterial Detection,” Biosensors and Bioelectronics 39(1): 282-288; Chowdhury, A. D., De. A., Chaudhuri, C. R., Bandyopadhyay, K., and Sen, P. 2012. “Label free Polyaniline Based Impedimetric Biosensor for Detection of E. coli 0157: H7 Bacteria,” Sensors and Actuators B Chemical 171:916-923; He. R.-X., Zhang, M., Tan, F., Leung, P. H. M., Zhao, X.-Z., and 3 others 2012. “Detection of Bacteria with Organic Electrochemical Transistors,” Journal of Materials Chemistry 22(41): 22072-220776; Zelada-Guillen, G. A., Sebastian- Avila, J. L., Blondeau, P., Riu. J., and Rius, F. X. 2012. “Label-free Detection of Staphylococcus aureus in Skin Using Real-time Potentiometric Biosensors Based on Carbon Nanotubes and Aptamers,” Biosensensors and Bioelectronics 31(1): 226-232; Xi. F. N., Gao, J. Q., Wang, J. N., and Wang, Z. X. 2011. “Discrimination and Detection of Bacteria with a Label-free Impedimetric Biosensor Based on Self-assembled Lectin Monolayer,” Journal of Electroanalytical Chemistry 656(1-2): 252-257; Zelada-Guillen, G. A., Bhosale, S. V., Riu, J., and Rius, F. X. 2010. “Real-time Potentiometric Detection of Bacteria in Complex Samples,” Analytical Chemistry 82(22): 9254-9260; Zelada-Guillen, G. A., Riu, J., Duzgun, A., and Rius F. X. 2009. “Immediate Detection of Living Bacteria at Ultralow Concentrations Using a Carbon Nanotube based Potentiometric Aptasensor,” Angewante Chemie International (International Edition in English-Hoboken, New Jersey) 48(40): 7334-7337; Serra, B., Gamella, M., Reviejo, A. J., and Pingarron, J. M. 2008. “Lectin-modified Piezoelectric Biosensors for Bacteria Recognition and Quantification,” Analytical and Bioanalytical Chemistry 391(5): 1853-1860; Zourob, M., Elwary, S., and Turner A. P. 2008. Principles of Bacterial Detection: Biosensors, Recognition Receptors and Microsystems, New York, New York: Springer Science and Business Media; Pang, P., Huang, S., Cai, Q., Yao. S., Zeng. K., and Grimes, C. A. 2007. “Detection of Pseudomonas aeruginosa Using a Wireless Magnetoelastic Sensing Device,” Biosensors and Bioelectronics 23(2): 295-299).

b. Viruses (see, for example, Abdul Ghani, M. A., Nordin, A. N., Zulhairee. M., Che Mohamad Nor. A., Shihabuddin Ahmad Noorden, M., and 3 others 2022. “Portable Electrochemical Biosensors Based on Microcontrollers for Detection of Viruses: A Review,” Biosensors (Basel, Switzerland) 12(8): 666; Mao. S., Fu. L., Yin, C., Liu, X., and Karimi-Maleh, H. 2022. “The Role of Electrochemical Biosensors in SARS-COV-2 [severe acute respiratory syndrome coronavirus 2] Detection: A Bibliometrics-based Analysis and Review,” Online, Royal Society of Chemistry Advances 12(35): 22592-22607; Wasfi, A., Awwad. F., Qamhieh, N., Al Murshidi. B., Palakkott, A. R., and Gelovani, J. G. 2022. “Real-time COVID-19 [corona virus disease 2019] Detection via Graphite Oxide-based Field-effect Transistor Biosensors Decorated with Pt/Pd [bimetallic platinum and palladium] Nanoparticles,” Online, (Nature Research) Scientific Reports 12(1): 18155; Xu, M., Li, Y., Lin, C., Peng. Y., Zhao, S., Yang, X., and Yang, Y. 2022 “Recent Advances of Representative Optical Biosensors for Rapid and Sensitive Diagnostics of SARS-COV-2 [severe acute respiratory syndrome coronavirus 2]. Online, Biosensors (Basel, Switzerland) 12(10): 862; Goud, K. Y., Reddy, K. K., Khorshed, A., Kumar, V. S., Mishra, R. K., and 4 others 2021. “Electrochemical Diagnostics of Infectious Viral Diseases: Trends and Challenges,” Biosensors and Bioelectronics 180:113112; Selvarajan, R. S., Gopinath, S. C. B., Zin, N. M., and Hamza, A. A. 2021. “Infection-mediated Clinical Biomarkers for a COVID-19 Electrical Biosensing Platform,” Online, Sensors (Basel, Switzerland) 21(11): 3829; Cesewski, E. and Johnson, B. N. 2020. “Electrochemical Biosensors for Pathogen Detection,” Biosensors and Bioelectronics 159:112214; Layqah, L. A. and Eissa, S. 2019. “An Electrochemical Immunosensor for the Corona Virus Associated with the Middle East Respiratory Syndrome Using an Array of Gold Nanoparticle-modified Carbon Electrodes,” Mikrochimica Acta (Vienna, Austria) 186(4): 224; Hai, W., Goda, T., Takeuchi, H., Yamaoka, S., Horiguchi, Y., Matsumoto, A., and Miyahara, Y. 2018. “Human Influenza Virus Detection Using Sialyllactose-functionalized Organic Electrochemical Transistors,” Sensors and Actuators B Chemical 260:635-641; Hou Y. H., Wang J. J., Jiang Y. Z., Lv C., Xia L., and 5 others 2018. “A Colorimetric and Electrochemical Immunosensor for Point-of-care Detection of Enterovirus 71,” Biosensors and Bioelectronics 99:186-192; Sayhi M., Querghi O., Belgacem K., Arbi M., Tepeli Y., and 5 others 2018. “Electrochemical Detection of Influenza Virus H9N2 Based on Both Immunomagnetic Extraction and Gold Catalysis Using an Immobilization-free Screen Printed Carbon Microelectrode,” Biosensors and Bioelectronics 107:170-177; Chand. R. and Neethirajan, S. 2017. “Microfluidic Platform Integrated with Graphene-gold Nano-composite Aptasensor for One-step Detection of Norovirus,” Biosensors and Bioelectronics 98:47-53; Chin, S. F., Lim, L. S., Pang, S. C., Sum, M. S. H., and Perera, D. 2017. “Carbon Nanoparticle Modified Screen Printed Carbon Electrode as a Disposable Electrochemical Immunosensor Strip for the Detection of Japanese Encephalitis Virus,” Microchimica Acta 184(2): 491-497; Colombini, E., Guzzo, I. Morolli, F., Longo, G., Russo, C., and 9 others 2017, Op cit., above in this section; Hai, W., Goda, T., Takeuchi, H., Yamaoka, S., Horiguchi, Y., Matsumoto, A., and Miyahara, Y. 2017. “Specific Recognition of Human Influenza Virus with PEDOT [Poly(3,4-ethylenedioxythiophene)] Bearing Sialic Acid-terminated Trisaccharides,” American Chemical Society Applied Materials and Interfaces 9(16): 14162-14170; Hwang H. J., Ryu M. Y., Park C. Y., Ahn J., Park H. G., and 4 others 2017. “Highly Sensitive and Selective Electrochemical Biosensor: Label-free Detection of Human Norovirus Using Affinity Peptide as Molecular Binder, Biosensors and Bioelectronics 87:164-170; Singh, R., Hong. S., and Jang J. 2017. “Label-free Detection of Influenza Viruses Using a Reduced Graphene Oxide-based Electrochemical Immunosensor Integrated with a Microfluidic Platform,” (Nature Research) Scientific Reports (London, England) 7:42771; Zhang, Q., Li, L., Qiao, Z., Lei. C., Fu. Y., and 4 others 2017. “Electrochemical Conversion of Fe₃O₄ Magnetic Nanoparticles to Electroactive Prussian Blue Analogues for Self-sacrificial Label Biosensing of Avian Influenza Virus H5N1,” Analytical Chemistry 89(22): 12145-12151; Attar, A., Mandli, J., Ennaji, M. M., and Amine, A. 2016. “Label-free Electrochemical Impedance Detection of Rotavirus Based on Immobilized Antibodies on Gold Sononanoparticles,” Electroanalysis 28(8) 1839-1846; Callaway, Z., Wang, Y., Zhang, B., Zhang, T., Costello, T. A., Slavik, M. F., and Li, Y. 2016. “A Portable Impedance Biosensing System for Rapid Detection of Avian Influenza Virus,” Transactions of the ASABE (Transactions of the American Society of Agricultural and Biological Engineers) 59(2): 421-428; Hushegyi, A., Pihikova, D., Bertok, T., Adam, V., Kizek, R., and Tkac, J. 2016. “Ultrasensitive Detection of Influenza Viruses with a Glycan-based Impedimetric Biosensor Biosensors and Bioelectronics 79:644-649; Kitajima, M., Wang, N., Tay, M. Q. X., Miao. J. M., and Whittle, A. J. 2016. “Development of a MEMS [microelectromechanical systems]-based Electrochemical Aptasensor for Norovirus Detection,” Micro and Nano Letters 11(10): 582-585; Rappo, U., Schuetz, A. N., Jenkins, S. G., Calfee, D. P., Walsh, T. J., and 3 others 2016. “Impact of Early Detection of Respiratory Viruses by Multiplex PCR [polymerase chain reaction] Assay on Clinical Outcomes in Adult Patients,” Journal of Clinical Microbiology 54(8): 2096; Altintas, Z., Gittens, M., Pocock, J., and Tothill, I. E. 2015. “Biosensors for Waterborne Viruses: Detection and Removal,” Biochimie (Paris, France) 115:144-154; Fu, Y., Lin J., Wang R., Jiao P., Li Y., Li Y., and 3 others 2015. “An Impedance Immunosensor Based on Low-cost Microelectrodes and Specific Monoclonal Antibodies for Rapid Detection of Avian Influenza Virus H5N1 in Chicken Swabs,” Biosensors and Bioelectronics 67:546-552; Hong, S. A., Kwon, J., Kim, D., and Yang, S. 2015. “A Rapid, Sensitive and Selective Electrochemical Biosensor with Concanavalin A for the Preemptive Detection of Norovirus,” Biosensors and Bioelectronics 64:338-344; Krejcova, L., Michalek. P., Merlos Rodrigo, M., Heger, Z., Krizkova, S., ad 4 others 2015. “Nanoscale Virus Biosensors: State of the Art” Nanobiosensors in Disease Diagnosis 4:47-66; Luna, D. M. N., Avelino, K. Y. P. S., Cordeiro, M. T., Andrade C. A. S., and Oliveira, M. D. L. 2015. “Electrochemical Immunosensor for Dengue Virus Serotypes Based on 4-mercaptobenzoic Acid Modified Gold Nanoparticles on Self-assembled Cysteine Monolayers,” Sensors and Actuators B Chemical 220:565-572; Zhou, C.-H., Wu, Z., Chen, J.-J., Xiong. C., Chen, Z., Pang, D.-W., and Zhang, Z.-L. 2015. “Biometallization-based Electrochemical Magnetoimmunosensing Strategy for Avian Influenza A (H7N9) Virus Particle Detection,” Chemistry, an Asian journal 10(6): 1387-1393; Fu. Y., Callaway, Z., Lum, J., Wang. R., Lin. J., and Li, Y. 2014. “Exploiting Enzyme Catalysis in Ultra-low Ion Strength Media for Impedance Biosensing of Avian Influenza Virus Using a Bare Interdigitated Electrode,” Analytical Chemistry 86(4): 1965-1971; Morton, M., Coupes, B., Robert, S. A., Johnson, S. L., Klapper, P. E., Vallely, P. J., and Picton, M. L 2014. Op cit, this section; Nidzworski, D., Pranszke, P., Grudniewska, M., Król, E., and Gromadzka, B. 2014. “Universal Biosensor for Detection of Influenza Virus,” Biosensors and Bioelectronics 59:239-242; Giamberardino, A., Labib. M., Hassan, E. M., Tetro, J. A., Springthorpe, S., and 3 others 2013. “Ultrasensitive Norovirus Detection Using DNA Aptasensor Technology, Online, PLoS [Public Library of Science] One 8(11): e79087; Liu, F., Kim, Y. H., Cheon, D. S., and Seo. T. S. 2013. “Micropatterned Reduced Graphene Oxide Based Field-effect Transistor for Real-time Virus Detection,” Sensors and Actuators B Chemical 186:252-257; Peh, A. E. and Li, S. F. 2013. “Dengue Virus Detection Using Impedance Measured across Nanoporous Alumina Membrane” Biosensors and Bioelectronics 42:391-396; Cheng, M. S., Ho, J. S., Tan, C. H., Wong, J. P., Ng. L. C., and Toh, C.-S. 2012. “Development of an Electrochemical Membrane-based Nanobiosensor for Ultrasensitive Detection of Dengue Virus,” Analytica Chimica Acta (Amsterdam, North Holland, Netherlands) 725:74-80; Lum, J., Wang R., Lassiter K., Srinivasan B., Abi-Ghanem D., and 5 others 2012. “Rapid Detection of Avian Influenza H5N1 [hemagglutinin type 5/neuraminidase type 1] Virus Using Impedance Measurement of Immuno-reaction Coupled with RBC Amplification” Biosensors and Bioelectronics 38(1): 67-73; Nguyen, B. T., Peh, A. E., Chee, C. Y., Fink, K., Chow V. T., Ng. M. M., and Toh, C. S. 2012. “Electrochemical Impedance Spectroscopy Characterization of Nanoporous Alumina Dengue Virus Biosensor,” Bioelectrochemistry 88:15-21; Hue, K. D., Tuan, T. V., Thi, H. T., Bich, C. T., Anh, H. H., Wills. B. A., and Simmons, C. P. 2011. “Validation of an Internally Controlled One-step Real-time Multiplex rt-PCR [reverse transcription-polymerase chain reaction] Assay for the Detection and Quantitation of Dengue Virus RNA in Plasma,” Journal of Virological Methods 177(2): 168-173; Lee. D., Chander, Y., Goyal, S. M., and Cui. T. 2011. “Carbon Nanotube Electric Immunoassay for the Detection of Swine Influenza Virus H1N1,” Biosensors and Bioelectronics 26(8): 3482-3487; Liu, F., Choi, K. S., Park, T. J., Lee, S. Y., and Seo. T. S. 2011. “Graphene-based Electrochemical Biosensor for Pathogenic Virus Detection,” Biochip Journal 5(2): 123-128; Wang. R., Li, Y., Mao, X., Huang. T., and Lu H. 2010. “Magnetic Bio-nanobeads and Nanoelectrode Based Impedance Biosensor for Detection of Avian Influenza Virus,” pages 214-217 in Institute of Electrical and Electronics Engineers International Conference on Nano/Molecular Medicine and Engineering: Nguyen, B. T., Koh, G., Lim, H. S., Chua, A. J., Ng. M. M., and Toh C. S. 2009. “Membrane-based Electrochemical Nanobiosensor for the Detection of Virus,” Analytical Chemistry 81(17): 7226-7234; Gupta, A., Akin, D., and Bashir. R. 2004. “Single Virus Particle Mass Detection Using Microresonators with Nanoscale Thickness,” Applied Physics Letters 84(11): 1976-1978).

c. Tumor cells, to include malignant and circulating (see, for example, Alawajii, R. A., Alsudani, Z. A. N., Biris, A. S., and Kannarpady, G. K. 2023. “Biosensor Design for the Detection of Circulating Tumor Cells Using the Quartz Crystal Resonator Technique,” Online, MDPI [Multidisciplinary Digital Publishing Institute] Biosensors (Basel, Switzerland) 13(4): 433; Dessale, M., Mengistu, G., and Mengist, H. M. 2022. “Nanotechnology: A Promising Approach for Cancer Diagnosis, Therapeutics, and Theragnosis,” Online, International Journal of Nanomedicine 17:3735-3749; Yadav, N., Dahiya, T., Chhillar, A. K., Rana, J. S., and Saini, H. M. 2022. “Nanotechnology in Cancer Diagnostics and Therapeutics: A Review,” Current Pharmaceutical Biotechnology 23(13): 1556-1568; Kartikasari, A. E. R., Huertas, C. S., Mitchell, A., and Piebanski, M. 2021. “Tumor-induced Inflammatory Cytokines and the Emerging Diagnostic Devices for Cancer Detection and Prognosis,” Online, Frontiers in Oncology 11:692142; Scott, J. I., Gutkin, S., Green, O., Thompson, E. J., Kitamura, T., Shabat, D., and Vendrell. M. 2021. “A Functional Chemiluminescent Probe for in Vivo Imaging of Natural Killer Cell Activity against Tumours,” Angewante Chemie (International Edition in English) 60(11): 5699-5703; Hajizadeh, F., Ardebili, S. M., Moornani, B., Masjedi. A., Atyabi, F. and 10 others 2020. “Silencing of HIF [Hypoxia Inducible Factor]-1alpha/CD73 (ecto-5′-nucleotidase] Axis by siRNA-loaded TAT [a cationic cell-penetrating peptide]-chitosan-spion Nanoparticles Robustly Blocks Cancer Cell Progression,” European Journal of Pharmacology 882:173235; Du. X., Zhang, Z., Zheng, X. Zhang, H., Dong. D., and 3 others 2020. “An Electrochemical Biosensor for the Detection of Epithelial-Mesenchymal Transition,” Nature Communications 11(1): 192; Fan, K., Zheng. Y., Li, D., Su. C., and Wang, G. 2020. “Research Progress on Electrochemical Biosensors for Breast Cancer Detection Based on Nanomaterials,” (in Chinese with English abstract at Pubmed) Zhongguo Yi Liao Qi Xie Za Zhi [Chinese Journal of Medical Instrumentation] 44(6): 525-531; Feiyun., C., Zhou, Z., and Zhou, H. S. 2020. “Review-Measurement and Analysis of Cancer Biomarkers Based on Electrochemical Biosensors,” Journal of the Electrochemical Society 167(3): 037525; Luo, J., Liang. D., Zhao, D., and Yang, M. 2020. “Photoelectrochemical Detection of Circulating Tumor Cells Based on Aptamer Conjugated Cu20 as Signal Probe,” Biosensors and Bioelectronics (Amsterdam, North Holland, Netherlands) 151:111976; Oliveira-Silva, R., Sousa-Jeronimo, M., Botequim, D., Silva, N. J. O., Paulo, P. M. R., and Prazeres, D. M. F. 2020. “Monitoring Proteolytic Activity in Real Time: A New World of Opportunities for Biosensors,” Trends in Biochemical Science 45(7): 604-618; Sharifi. M., Hasan, A., Attar, F., Taghizadeh, A., and Falahati, M. 2020. “Development of Point-of-care Nanobiosensors for Breast Cancers Diagnosis,” Talanta 217:121091; Zhang, Z., Li, Q., Du, X., and Liu, M. 2020. “Application of Electrochemical Biosensors in Tumor Cell Detection,” Thoracic Cancer 11(4): 840-850; Cao, Y., Dai, Y., Chen, H., Tang. Y., Chen, X., and 3 others 2019. “Integration of Fluorescence Imaging and Electrochemical Biosensing for Both Qualitative Location and Quantitative Detection of Cancer Cells,” Biosensors and Bioelectronics (Amsterdam, North Holland) 130:132-138; Chaturvedi, V. K., Singh, A., Singh, V. K., Singh, M. P. 2019. “Cancer Nanotechnology: A New Revolution for Cancer Diagnosis and Therapy,” Current Drug Metabolism 20(6): 416-429; Li, N., Zhong. D., Chen, H., Huang. T., Hou, P., and 4 others 2019. “The Utility of Folate Receptor-positive Circulating Tumor Cell in Cancer Diagnosis in the Elderly Population,” Cancer Management and Research 11:4097-4107; Sharifi. M., Avadi, M. R., Attar, F., Dashtestani, F., Ghorchian, H., and 3 others 2019. “Cancer Diagnosis Using Nanomaterials Based Electrochemical Nanobiosensors,” Biosensors and Bioelectronics (Amsterdam, North Holland) 126:773-784; Sun, D., Lu, J., Zhang. L., and Chen, Z. 2019. “Aptamer-based Electrochemical Cytosensors for Tumor Cell Detection in Cancer Diagnosis: A review,” Analytica Chimica Acta (Amsterdam, North Holland, Netherlands) 1082:1-17; Wang. Y., Chang, K., Yang, C., Li, S., Wang. L. and 6 others 2019. “Highly Sensitive Electrochemical Biosensor for Circulating Tumor Cells Detection via Dual-aptamer Capture and Rolling Circle Amplification Strategy,” Journal of Biomedical Nanotechnology 15(7): 1568-1577; Wang, S.-S., Zhao, X.-P., Liu, F.-F., Younis, M. R., Xia, X.-H., and Wang, C. 2019. “Direct Plasmon-enhanced Electrochemistry for Enabling Ultrasensitive and Label-free Detection of Circulating Tumor Cells in Blood,” Analytical Chemistry 91(7): 4413-4420; Xu, J., Wang, X., Yan. C., and Chen, W. 2019. “A Polyamidoamine Dendrimer-based Electrochemical Immunosensor for Label-free Determination of Epithelial Cell Adhesion Molecule-expressing Cancer Cells,” Online, Sensors (Basel, Switzerland) 19(8): 1879; Cai. C., Guo, Z., Cao. Y., Zhang, W., and Chen, Y. 2018. “A Dual Biomarker Detection Platform for Quantitating Circulating Tumor DNA (ctDNA),” Online, Nanotheranostics (Sydney, Australia) 2(1): 12-20; Chen, M. and Xie, S. 2018. “Therapeutic Targeting of Cellular Stress Responses in Cancer,” Thoracic Cancer 9(12): 1575-1582; Liu, J.-X., Bao, N., Luo, X., and Ding. S.-N. 2018. “Nonenzymatic Amperometric Aptamer Cytosensor for Ultrasensitive Detection of Circulating Tumor Cells and Dynamic Evaluation of Cell Surface N-glycan Expression,” American Chemical Society Omega 3(8): 8595-8604; Liu, M., Xu. Y., Huang, C., Jia. T., Zhang, X., Yang, D.-P., and Jia, N. 2018. “Hyaluronic Acid-grafted Three-dimensional MWCNT [multiwall carbon nanotube] Array as Biosensing Interface for Chronocoulometric Detection and Fluorometric Imaging of CD44-Overexpressing Cancer Cells,” Mikrochimica Acta (Vienna, Austria) 185(7): 338; Sun, D., Lu J., Luo, Z., Zhang, L., Liu, P., and Chen, Z. 2018. “Competitive Electrochemical Platform for Ultrasensitive Cytosensing of Liver Cancer Cells by Using Nanotetrahedra Structure with Rolling Circle Amplification,” Biosensors and Bioelectronics (Amsterdam, North Holland, Netherlands) 120:8-14; Sun, S. and Zhou, J. 2018. “Molecular Mechanisms Underlying Stress Response and Adaptation,” Thoracic Cancer 9(2): 218-227; Tang, S., Shen, H., Hao, Y. Huang, Z. Tao, Y., and 4 others 2018. “A Novel Cytosensor Based on Pt@ Ag Nanoflowers and AuNPs/Acetylene Black for Ultrasensitive and Highly Specific Detection of Circulating Tumor Cells,” Biosensors and Bioelectronics (Amsterdam, North Holland) 104:72-78; Tang, Y.-H., Lin, H.-C., Lai, C.-L., Chen, P.-Y., and Lai. C.-H. 2018. “Mannosyl Electrochemical Impedance Cytosensor for Label-free MDA-MB-231 Cancer Cell Detection,” Biosensors and Bioelectronics (Amsterdam, North Holland) 116:100-107; Wu, X., Xiao. T., Luo, Z., He. R., Cao, Y. and 3 others 2018. “A Micro-/Nano-chip and Quantum Dots-based 3D Cytosensor for Quantitative Analysis of Circulating Tumor Cells,” Journal of Nanobiotechnology 16(1): 65; Zhang. L., Xu. Z., Kang. Y., and Xue, P. 2018. “Three-dimensional Microfluidic Chip with Twin-layer Herringbone Structure for High [sic Highly] Efficient Tumor Cell Capture and Release via Antibody-conjugated Magnetic Microbeads,” Electrophoresis (Weinheim, Germany) 39(12): 1452-1459; Aydin, E. B. and Sezginturk, M. K. 2017. “A Sensitive and Disposable Electrochemical Immunosensor for Detection of SOX2, a Biomarker of Cancer,” Talanta (Amsterdam, North Holland, Netherlands) 172:162-170; Huang, X., Liu, Y., Yung. B., Xiong. Y., and Chen, X. 2017. “Nanotechnology-enhanced No-wash Biosensors for in Vitro Diagnostics of Cancer,” American Chemical Society Nano 11(6): 5238-5292; Kwon, E. J., Dudani, J. S., and Bhatia, S. N. 2017. “Ultrasensitive Tumour-penetrating Nanosensors of Protease Activity,” Nature Biomedical. Engineering 1:0054; Wang. B., Akiba, U., and Anzai, J. I. 2017. “Recent Progress in Nanomaterial-based Electrochemical Biosensors for Cancer Biomarkers: A Review,” Online, Molecules 22(7): 1048; Yu. C. C., Ho, B. C., Juang. R. S., Hsiao, Y.-S., R Venkata, R. N., and 5 others 2017. “Poly(3,4-ethylenedioxythiophene)-based Nanofiber Mats as an Organic Bioelectronic Platform for Programming Multiple Capture/Release Cycles of Circulating Tumor Cells,” American Chemical Society Applied Materials and Interfaces 9(36): 30329-30342; Zhao, F. 2017. “Electrochemical Techniques for the Detection of Cancer Cells and Cell-surface Glycan Expression: A Mini Review,” International Journal of Electrochemical Science (Bor, Serbia) 12(8): 7580-7596; Tao, Z., Dang, X., Huang, X., Muzumdar, M. D., Xu. E. S. and 10 others 2017. “Early Tumor Detection Afforded by in Vivo Imaging of Near-infrared II Fluorescence,” Biomaterials (Amsterdam, North Holland, Netherlands) 134:202-215; Fan, Z., Sun, L., Huang, Y., Wang, Y., and Zhang, M. 2016. “Bioinspired Fluorescent Dipeptide Nanoparticles for Targeted Cancer Cell Imaging and Real-time Monitoring of Drug Release,” Nature Nanotechnology 11(4): 388-394; Liu, J., Cui, M., Niu. L., Zhou, H., and Zhang, S. 2016. “Enhanced Peroxidase-like Properties of Graphene-Hemin-composite Decorated with Au Nanoflowers as Electrochemical Aptamer Biosensor for the Detection of K562 Leukemia Cancer Cells,” Chemistry 22(50): 18001-18008; Sun, D., Lu, J., Zhong, Y., Yu, Y., Wang. Y., Zhang, B., and Chen, Z. 2016. “Sensitive Electrochemical Aptamer Cytosensor for Highly Specific Detection of Cancer Cells Based on the Hybrid Nanoelectrocatalysts and Enzyme for Signal Amplification,” Biosensors and Bioelectronics (Amsterdam, North Holland) 75:301-307; Whitley, M. J. Cardona, D. M., Lazarides, A. L., Spasojevic, I., Ferrer, J. M. and 21 others 2016. “A Mouse-human Phase 1 Co-clinical Trial of a Protease- activated Fluorescent Probe for Imaging Cancer,” Science Translational Medicine 8(320): 320ra4; Kwong, G. A., Dudani, J. S., Carrodeguas, E., Mazumdar, E. V., Zekavat, S. M., and Bhatia, S. N. 2015. “Mathematical Framework for Activity-based Cancer Biomarkers,” Online, Proceedings of the National Academy of Sciences of the United States of America 112(41): 12627-12632.; Tsuchiya, N., Sawada, Y., Endo, I., Saito, K., Uemura, Y., and Nakatsura, T. 2015. “Biomarkers for the Early Diagnosis of Hepatocellular Carcinoma, World Journal of Gastroenterology 21(37): 10573-10583; Costa, C., Abal. M., Lopez-Lopez, R., and Muinelo-Romay, L. 2014. “Biosensors for the Detection of Circulating Tumour Cells,” Sensors (Basel, Switzerland) 14(3): 4856-4875; Wan, Y., Zhou, Y. G., Poudineh, M. Safaei. T. S., Mohamadi. R. M., Sargent, E. H., and Kelley, S. O. 2014. “Highly Specific Electrochemical Analysis of Cancer Cells Using Multi-nanoparticle Labeling,” Angewandte Chemie (International Edition in English) 53(48): 13145-13149; Liu, H., Xu. S., He, Z., Deng. A., and Zhu, J. J. 2013 “Supersandwich Cytosensor for Selective and Ultrasensitive Detection of Cancer Cells Using Aptamer-DNA Concatamer-quantum Dots Probes,” Analytical Chemistry 85(6): 3385-3392; Yeong Won, J., Choi, J. W., and Min, J. 2013. “Micro- fluidic Chip Platform for the Characterization of Breast Cancer Cells Using Aptamer-assisted Immunohistochemistry,” Biosensors and Bioelectronics (Amsterdam, North Holland, Netherlands) 40(1): 161-166; Kumeria, T., Kurkuri, M. D., Diener, K. R., Parkinson, L., and Losic D. 2012. “Label-free Reflectometric Interference Microchip Biosensor Based on Nanoporous Alumina for Detection of Circulating Tumour Cells,” Biosensensors and Bioelectronics (Amsterdam, North Holland) 35(1): 167-173; Hori, S. S. and Gambhir, S. S. 2011. “Mathematical Model Identifies Blood Biomarker-based Early Cancer Detection Strategies and Limitations,” Science Translational Medicine 3(109): 109ral 16-109ral 16; Sugahara, K. N., Teesalu, T., Karmali, P. P., Kotamraju, V. R., Agemy, L., and 4 others 2009. “Tissue-penetrating Delivery of Compounds and Nanoparticles into Tumors,” Cancer Cell (Cambridge, Massachusetts) 16(6): 510-520; Park, J.-H., von Maltzahn, G., Zhang. L., Schwartz, M. P., Ruoslahti, F., Bhatia, S. N., and Sailor, M. J. 2008. “Magnetic Iron Oxide Nanoworms for Tumor Targeting and Imaging,” Advanced. Materials (Deerfield Beach, Florida) 20(9): 1630-1635; Wu. T., Zhang, Z., and Zhou Y. (assignors to Genentech Corp.) 2007. “Compositions and Methods for the Diagnosis and Treatment of Tumor,” U.S. Patent Applications US20070224201A1; Smyth, M. J., Hayakawa, Y., Takeda, K., and Yagita H. 2002. “New Aspects of Natural-killer-cell Surveillance and Therapy of Cancer,” Nature Reviews. Cancer 2(11): 850-861).

Efforts have been made to devise materials that will seek out and penetrate a tumor-some, such as Wang Y., Wang. R., Chen, L., Chen, L., Zheng, Y, and 4 others 2022 included below-incorporating magnetically susceptible matter likely beneficial when combined with the scheme depicted in FIGS. 14A and 14B (see, for example, Li, C. H., Chang, Y. C., Hsiao, M., and Chan, M. H. 2022. “Ultrasound and Nanomedicine for Cancer-targeted Drug Delivery: Screening, Cellular Mechanisms, and Therapeutic Opportunities,” Online, MDPI [Multidisciplinary Digital Publishing Institute] Pharmaceutics (Basel, Switzerland) 14(6): 1282; Fahmi, A., Abdur-Rahman, M., Mahareek, O., and Shemis, M. A., 2022. “Synthesis, Characterization, and Cytotoxicity of Doxorubicin-loaded Polycaprolactone Nanocapsules as Controlled Anti-hepatocellular Carcinoma Drug Release System,” Online, BioMed Central Chemistry 16(1): 85; Fatima, M., Sheikh, A., Abourehab, M. A. S., and Kesharwani, P. 2022. “Advancements in Polymeric Nanocarriers to Mediate Targeted Therapy against Triple-negative Breast Cancer,” Online, MDPI [Multidisciplinary Digital Publishing Institute] Pharmaceutics (Basel, Switzerland) 14(11): 2432; Kanugo, A., Gautam, R. K. and Kamal, M. A. 2022. “Recent Advances in Nanotechnology in the Diagnosis and Therapy of Triple Negative Breast Cancer,” Current Pharmaceutical Biotechnology 23(13): 1581-1595; Wang Y., Wang. R., Chen, L., Chen, L., Zheng, Y, and 4 others 2022. “Enhanced Tumor Penetration for Efficient Chemotherapy by a Magnetothermally Sensitive Micelle Combined with Magnetic Targeting and Magnetic Hyperthermia,” Online, Frontiers in Pharmacology (Lausanne, Switzerland) 13:1045976; Zhang, J., Zhang, T., and Gao, J. 2022. “Biocompatible Iron Oxide Nanoparticles for Targeted Cancer Gene Therapy: A Review,” Online, MDPI [Multidisciplinary Digital Publishing Institute] Nanomaterials (Basel, Switzerland) 12(19): 3323; Kim, C., Guo, Y., Velalopoulou, A., Leisen, J., Moammarry, A., and 4 others 2021. “Closed-loop Trans-skull Ultrasound Hyperthermia Leads to Improved Drug Delivery from Thermosensitive Drugs and Promotes Changes in Vascular Transport Dynamics in Brain Tumors,” Online, Theranostics (Wyoming, New South Wales, Australia) 11(15): 7276-7293; Chen, X., Gole, J., Gore. A., He, Q. Lu, M., and 27 others 2020. “Non-invasive Early Detection of Cancer Four Years before Conventional Diagnosis Using a Blood Test,” Online, Nature Communications (London, England) 11(1): 3475; Ruoslahti, E. 2017. “Tumor Penetrating Peptides for Improved Drug Delivery,” Online, Advanced Drug Delivery Reviews 110-111:3-12; Khaledian, M. Nourbakhsh, M. S., Saber, R., Hashemzadeh, H., and Darvishi, M. H., 2020. “Preparation and Evaluation of Doxorubicin-loaded PLA-PEG-FA [polylactide-polyethylene glycol-folic acid] Copolymer Containing Superparamagnetic Iron Oxide Nanoparticles (SPIONs) for Cancer Treatment Combination Therapy with Hyperthermia and Chemotherapy,” Online, International Journal of Nanomedicine 15:6167-6182;). Park, J. H., von Maltzahn, G., Zhang, L., Schwartz, M. P., Ruoslahti. E., Bhatia, S. N., and Sailor, M. J. 2008. “Magnetic Iron Oxide Nanoworms for Tumor and Imaging,” Online, Advanced Materials (Deerfield Beach, Florida) 20(9): 1630-1635).

d. Sundry infectious pathogens, infection, and symptoms thereof (see, for example, Sivakumar, R. and Lee, N. Y. 2022. “Recent Advances in Airborne Pathogen Detection Using Optical and Electrochemical Biosensors,” Online, Analytica Chimica Acta 1234:340297; Ramanavicius, S., Jagminas, A., and Ramanavicius, A. 2021. “Advances in Molecularly Imprinted Polymers Based Affinity Sensors (Review),” Polymers (Basel, Switzerland) 13(6): 974; Shaban, S. M. and Kim, D. H. 2021. “Recent Advances in Aptamer Sensors,” Sensors (Basel, Switzerland) 21(3) 979; Cesewski, E. and Johnson, B. N. 2020. “Electrochemical Biosensors for Pathogen Detection,” Biosensors and Bioelectronics (Amsterdam, North Holland, Netherlands) 159:112214; Cui, F., Zhou, Z., and Zhou, H. S. 2020. “Molecularly Imprinted Polymers and Surface Imprinted Polymers Based Electrochemical Biosensor for Infectious Diseases,” Sensors (Basel, Switzerland) 20(4): 996; Kim, J. J., Stafford, G. R., Beauchamp. C., and Kim, S. A. 2020. “Development of a Dental Implantable Temperature Sensor for Real-time Diagnosis of Infectious Disease,” Online, Sensors (Basel, Switzerland) 20(14): 3953; Zhang, Z., Cong. Y., Huang, Y., and Du, X. 2019. “Nanomaterials-based Electrochemical Immunosensors,” Micromachines 10(6) 1-8; Amiri, M., Bezaatpour, A., Jafari, H., Boukherroub. R., and Szunerits, S. 2018. “Electrochemical Methodologies for the Detection of Pathogens,” American Chemical Society Sensors 3(6): 1069-1086; Du, X. and Zhou, J. 2018. “Application of Biosensors to Detection of Epidemic Diseases in Animals,” Research in Veterinary Science 118:444-448; Gui. R., Jin, H., Guo, H., and Wang, Z. 2018. “Recent Advances and Future Prospects in Molecularly Imprinted Polymers-based Electrochemical Biosensors,” Biosensors and Bioelectronics (Amsterdam, North Holland) 100:56-70; Jing. A., Zhang, C., Liang, G., Feng. W., Tian, Z., and Jing. C. 2018. “Hyaluronate-functionalized Graphene for Label-free Electrochemical Cytosensing,” Micromachines 9(12): 1-11; Campuzano, S., Yañez- Sedeño, P., and Pingarrón, J. M. 2017. “Molecular Biosensors for Electrochemical Detection of Infectious Pathogens in Liquid Biopsies: Current Trends and Challenges,” Sensors (Basel, Switzerland) 17(11): 2533; Slomovic, S., Pardee, K., and Collins, J. J. 2015. “Synthetic Biology Devices for in Vitro and in Vivo Diagnostics,” Online, Proceedings of the National Academy of Sciences of the United States of America 112(47): 14429-14435; Sin, M. L. Y. Mach, K. E., Wong, P. K., and Liao, J. C. 2014. “Advances and Challenges in Biosensor-based Diagnosis of Infectious Diseases,” Expert Review of Molecular Diagnostics 14(2): 225-244; Fuchs, Y., Soppera. O., and Haupt, K. 2012. “Photopolymerization and Photostructuring of Molecularly Imprinted Polymers for Sensor Applications—A Review,” Analytica Chimica Acta 717:7-20).

e. Graft organ rejection analytes and immunosensors. Recently, pan-, rather than specific-organ allograft dysfunction as indicative of rejection has emerged to “establish the capacity for machine learning models to learn across organs and present a transcriptomic transplant resource that can be employed to develop pan-organ biomarkers of allograft dysfunction,” (Robertson, H., Kim, H. J., Li, J., Robertson, N., Robertson, P., and 8 others 2024. “Decoding the Hallmarks of Allograft Dysfunction with a Comprehensive Pan-organ Transcriptomic Atlas,” Online, Nature Medicine (New York, New York) Jun. 18, 2024 [in press])(see also Abedini-Nassab. R., Taheri. F., Emamgholizadeh, A., and Naderi-Manesh, H. 2024. “Single-cell RNA Sequencing in Organ and Cell Transplantation,” Online, MDPI [Multidisciplinary Digital Publishing Institute] Biosensors (Basel, Switzerland) 14(4): 189; Pan, J., Ye, F., Li, H., Yu, C., Mao, J., and 9 others 2024. “Dissecting the Immune Discrepancies in Mouse Liver Allograft Tolerance and Heart//kidney Allograft Rejection,” Online, Cell Proliferation (Oxford, England) 57(3): e13555; Bei, K. F., Moshkelgosha. S., Liu, B. J., and Juvet, S. 2023. “Intragraft Regulatory T Cells in the Modern Era: What Can High-dimensional Methods Tell Us about Pathways to Allograft Acceptance?,” Online, Frontiers in Immunology (Lausanne, Switzerland) 14:1291649; Lim, J. H., Chung, B. H., Lee, S. H., Jung, H. Y., Choi, J. Y., and 4 others 2022. “Omics-based Biomarkers for Diagnosis and Prediction of Kidney Allograft Rejection,” Online, Korean Journal of Internal Medicine (Seoul, South Korea) 37(3): 520-533; Mederacke, Y. S., Nienen, M., Jarek, M., Geffers. R., Hupa-Breier, K., and 7 others 2021. “T Cell Receptor Repertoires within Liver Allografts are Different to Those in the Peripheral Blood,” Journal of Hepatology (Amsterdam, North Holland, Netherlands)(5): 1167-1175; Madariaga, M. L., Kreisel, D., and Madsen, J. C. 2015. “Organ-specific Differences in Achieving Tolerance,” Online, Current Opinion in Organ Transplantation (Hagerstown, Maryland)(4): 392-399; Moller, F., Hoyt. G., Farfan, F., Stammes. V. A., and Clayberger, C. 1993. “Cellular Mechanisms Underlying Differential Rejection of Sequential Heart and Lung Allografts in Rats,” Transplantation (Hagerstown, Maryland) 55(3): 650-655).

Otherwise, references cited pertain to diagnosis of rejection following transplantation and not the prediction thereof. Some references listed presume that once fully implantable prosthetic disorder response systems are available, implantable sensors will be developed to detect disease and adverse reaction-related analytes with increasing specificity. Urinalysis has been omitted as extracorporeal (see, for example, Allison, S. J. 2019. “Nanosensors Enable Early Detection of Acute T Cell-mediated Rejection of Transplants,” Nature Reviews Nephrology 15(5): 253; Chong, A. S. 2019. “Urinary Nanosensors of Early Transplant Rejection,” Nature Biomedical Engineering 3(4): 251-252; Mac, Q. D., Mathews, D. V., Kahla, J. A., Stoffers. C. M., Delmas. O. M., and 3 others 2019. “Non-invasive Early Detection of Acute Transplant Rejection via Nanosensors of Granzyme-B Activity,” Nature Biomedical Engineering 3(4): 281-291; Chin. L.-Y., Carroll. C., Raigani, S., Detelich, D. M., Tessier, S. N., and 6 others 2018. “Ex Vivo Perfusion-based Engraftment of Genetically Engineered Cell Sensors into Transplantable Organs,” Online, PLoS [Public Library of Science] One 14(12): e0225222; Dharnidharka, V. R. and Malone, A. 2018. “Biomarkers to Detect Rejection after Kidney Transplantation,” Pediatric Nephrology 33(7): 1113-1122; Holt, B. A., Mac, Q. D., and Kwong, G. A. 2018. “Nanosensors to Detect Protease Activity in Vivo for Noninvasive Diagnostics,” Journal of Visualized Experiments 137:57937; Haas, M., Loupy, A., Lefaucheur. C., Roufosse, C., Glotz, D., and 24 others 2017. “Revised Diagnostic Criteria for Chronic Active T Cell-mediated Rejection, Antibody-mediated Rejection, and Prospects for Integrative Endpoints for Next-generation Clinical Trials,” (Report of the Banff Kidney Meeting). American Journal of Transplantation 18(2): 293-307; Nasr. M., Sigdel. T., and Sarwala, M. 2016. “Advances in Diagnostics for Transplant Rejection,” Expert Reviews in Molecular Diagnosis 16(10): 1121-1132; Ahn, K. T., Choi, J. O., Lee, G. Y., Park, H. D., and Jeon, E. S. 2015. “Usefulness of High-sensitivity Troponin I for the Monitoring of Subclinical Acute Cellular Rejection after Cardiac Transplantation,” Transplantation Proceedings 47(2): 504-510: De Vlaminck, I., Martin, L., Kertesz, M., Patel, K., Kowarsky, M., and 11 others 2015. “Noninvasive Monitoring of Infection and Rejection after Lung Transplantation,” Online, Proceedings of the National Academy of Sciences of the United States of America 112(43): 13336-13341; Germani, G., Rodriguez-Castro, K., Russo, F. P., Senzolo, M., Zanetto, A., Farrarse, A., and Burra, P. 2015. “Markers of Acute Rejection and Graft Acceptance in Liver Transplantation,” World Journal of Gastroenterology 21(4): 1061-1065; Grenzi. P. C., Campos, E. F., Silva, H. T., Felipe, C. R., Franco, M. F., and 3 others 2015. “Post-transplant Soluble CD30 Levels are Associated with Early Subclinical Rejection in Kidney Transplantation,” Transplant Immunology 32(2): 61-65; Amirzargar, M. A., Amirzargar, A., Basiri, A., Hajiloo, M., Roshanaei, G., and 3 others 2014. “Early Post-transplant Immune Monitoring Can Predict Long-term Kidney Graft Survival: Soluble CD30 Levels, Anti-HLA Antibodies and IgA-anti-Fab Autoantibodies,” Human Immunology 75(1): 47-58; De Vlaminck, I., Valantine, H. A., Snyder, T. M., Strehl, C., Cohen, G., Luikart, H., Neff, N. F., and 5 others 2014. “Circulating Cell-free DNA Enables Noninvasive Diagnosis of Heart Transplant Rejection,” Science Translational Medicine 6(241): 24 Ira77; Govender, L., Pascual. M., and Golshayan, D. 2014. “Potential and Limitations of Regulatory T-cell Therapy in Solid Organ Transplantation,” Expert Reviews in Clinical Immunology 10(9): 1197-1212; Huang, H., Xu, X., Yao. C., Cai. M., Qian, Y., Wang. X., and Shi, B. 2014. “Serum Levels of CXCR3 Ligands Predict T Cell-mediated Acute Rejection after Kidney Transplantation,” Molecular Medicine Reports 9(1): 45-50; Macher, H. C., Suárez-Artacho, G., Guerrero, J. M., Gómez-Bravo, M. A., Alvarez-Gomez, s., and 3 others 2014. “Monitoring of Transplanted Liver Health by Quantification of Organ-specific Genomic Marker in Circulating DNA from Receptor,” Online, PLoS [Public Library of Science] One (San Francisco, California) 9(12): 1-18/e113987; Mori, D. N., Kreisel, D., Fullerton, J. N., Gilroy, D. W. and Goldstein, D. R 2014. “Inflammatory Triggers of Acute Rejection of Organ Allografts,” Immunological Reviews 258(1): 132-144; Patel, P. C., Hill, D. A., Ayers, C. R., Lavingia, B., Kaiser, P. and 10 others 2014. “High-sensitivity Cardiac Troponin I Assay to Screen for Acute Rejection in Patients with Heart Transplant,” Circulation. Heart Failure 7(3): 463-469; Roedder, S., Sigdel. T., Salomonis, N., Hsieh, S., Dai, H., and 15 others 2014. “The kSORT [Kidney Solid Organ Response Test] Assay to Detect Renal Transplant Patients at High Risk for Acute Rejection: Results of the Multicenter AART [Assessment of Acute Rejection in Renal Transplantation] Study,” Online, PLoS [Public Library of Science] Medicine 11(11): e1001759; Verhelst, X. P., Troisi. R. I., Colle, I., Geerts, A., and van Vlierberghe, H. 2013. “Biomarkers for the Diagnosis of Acute Cellular Rejection in Liver Transplant Recipients: A Review,” Hepatology Research 43(2): 165-178; Moreau, A., Varey, E., Anegon, I. and Cuturi, M-C. 2013. “Effector Mechanisms of Rejection,” Cold Spring Harbor Perspectives in Medicine 3(11): a015461; De Serres, S. A., Mfarrej. B. G., Grafals. M., Riella, L. V., Magee, C. N., and 5 others 2012. “Derivation and Validation of a Cytokine-[IL-6] based Assay to Screen for Acute Rejection in Renal Transplant Recipients,” Clinical Journal of the American Society of Nephrology 7(6): 1018-1025; Fabrega. E., Lopez-Hoyos, M., San Segundo, D., Casafont, F., Angel Mieses, M., Sampedro, B., and Pons-Romero, F. 2012. “Serum Levels of Interleukin-9 during Acute Rejection in Liver Transplantation,” Transplantation Proceedings 44(6): 1533-1535; Sigdel. T. K., Gao, X., and Sarwal, M. M. 2012. “Protein and Peptide Biomarkers in Organ Transplantation,” Biomarkers in Medicine 6(3): 259-271; Sigdel, T. K. and Sarwal, M. M. 2012. “Cell-free DNA as a Measure of Transplant Injury,” Clinical Transplants 201-206; Josephson, M. A. 2011. “Monitoring and Managing Graft Health in the Kidney Transplant Recipient,” Clinical Journal of the American Society of Nephrology 6(7): 1774-1780; Martinu, T., Pavlisko, E. N., Chen, D. F., and Palmer, S. M. 2011. “Acute Allograft Rejection: Cellular and Humoral Processes,” Clinics in Chest Medicine 32(2): 295-310; Muñoz-Esparza, C., Garrido, I. P., Blanco, R., Casas. T., Gonzalez-Canovas. C., and 5 others 2011. “Usefulness of High Sensitivity Troponin T Assay in Detecting Acute Allograft Rejection after Heart Transplantation, in Spanish with English abstract at Pubmed, Revista Española de Cardiologia 64(12): 1109-1113; Snyder, T. M., Khush, K. K., Valantine, H. A., and Quake, S. R. 2011. “Universal Noninvasive Detection of Solid Organ Transplant Rejection,” Online, Proceedings of the National Academy of Sciences of the United States of America 108(15): 6229-6234; Choy, J. C. 2010. “Granzymes and Perforin in Solid Organ Transplant Rejection,” Cell Death and Differentiation 17(4): 567-576; Fabrega, E., Lopez-Hoyos. M., Segundo, D. S., Casafont. F., and Pons-Romero, F. 2009. “Changes in the Serum Levels of Interleukin-17/Interleukin-23 during Acute Rejection in Liver Transplantation,” Liver Transplantation 15(6): 629-633; Nankivell. B. J. and Alexander, S. I. 2010. “Rejection of the Kidney Allograft,” New England Journal of Medicine 363(15) 1451-1462; Gwinner, W. 2007. “Renal Transplant Rejection Markers,” World Journal of Urology 25(5): 445-455; LaRosa, D. F., Rahman, A. H. and Turka, L. A. 2007. “The Innate Immune System in Allograft Rejection and Tolerance,” Journal of Immunology (Baltimore, Maryland) 178(12): 7503-7509).

f. Inflammation. Inflammation in general use is a nonspecific term used to denote swelling and pain which occur under numerous circumstances to include autoimmune disease and following exposure to toxic chemicals, for example. Numerous conditions unrelated to organ transplantation and the implantation of medical devices involve inflammation, but that relevant to the content here generally relates to tissue injury primarily caused by an immune foreign body response or surgical trauma rather than an infection, for example.

While other relevant categories include subsections e. Graft Organ Rejection Analytes and Immunosensors, and h. Coagulation, Thrombus, above, a separate subsection n. Adverse drug reactions to drugs is provided below for this relatively distinct cause of inflammation in relation to drugs used during procedures addressed here (see, for example, Kommu. S. and Whitfield, P. 2024. “Adverse Drug Reactions,” Online, Treasure Island Florida: StatPearls Publishing Co.).

Other sensor types such as to signal an arrhythmia are pertinent as well but not included in this section. Both the response to the implantation of diagnostic and therapeutic apparatus or a foreign body reaction and the implantation of an allogeneic organ represent immune reactions which include inflammation. To distinguish among inflammation, graft versus host rejection, and a cytokine storm is necessary to list references as focused on one such factor but is often, if unavoidably, fictitious, these phenomena largely cooriginal (see, for example, Ferrara, J. L., Abhyankar, S., and Gilliland, D. G. 1993. “Cytokine Storm of Graft-versus-host Disease: A Critical Effector Role for Interleukin-1,” Transplantation Proceedings 25(1 Part 2): 1216-1217).

The distinction between cytokine, transplant rejection, and pathogen response sensors is tenuous at best. Addressed above under subsection c. Tumor Cells, to Include Malignant and Circulating, inflammatory cytokines are induced by cancer. When the responsive analyte or analytes appear indistinguishable, this leaves a resolution as to the etiology to the use of collateral sensors, more highly selective sensors, and/or a close clinical diagnosis of the signs and symptoms but allows a considerable reduction in the number and type of sensors needed.

The same reservation must be expressed in relation to the detection of a viral infection and a cytokine storm, which might with equal justification have been listed under subsection b, above, entitled Viruses or subsection i. below, entitled Stress and Crisis Detecting Sensors (see, for example, Russell, S. M., Alba-Patino, A., Baron, E., Borges, M., Gonzalez-Freire, M., and de la Rica, R. 2020. “Biosensors for Managing the COVID-19 Cytokine Storm: Challenges Ahead,” Online, American Chemical Society Sensors 5(6): 1506-1513).

Not just the term inflammation but its primary diagnostic measures are nonspecific. Induced by virtually every condition of disease to include infection, trauma, and autoimmune disease, the sensing of inflammation, usually through detecting an increase in c-reactive protein and/or an elevated erythrocyte sedimentation rate, are the most common diagnostic tests for identifying inflammation which if advanced, indicates a need for medical intervention. Both tests confirm the presence of inflammation but are not definitive as to the cause, thus necessitating other sensors to identify the source. Sensors for the present purpose are preferably implantable for an indefinite period and therefore generally exclude biosensors which incorporate a biologically sensitive component when that component is not nonintrusively replenishable. Wearable sensors are addressed above in this section.

Sensor and biosensor technology for the detection of inflammation and internal conditions associated with and indicative of inflammation, such as elevated temperature, pressure, and force, has been advancing (see, for example, Salvo, P., Dini, V., Kirchhain, A., Janowska, A., Oranges. T., and 4 others 2017, Op cit. in this subsection; Ledet, E. H., D'Lima, D., Westerhoff, P., Szivek, J. A., Wachs. R. A., and Bergmann, G. 2012. “Implantable Sensor Technology: From Research to Clinical Practice,” Journal of the American Academy of Orthopedic Surgery 20(6): 383-392; Sinha, N., Ma. J., and Yeow. J. T. 2006. “Carbon Nanotube-based Sensors,” Journal of Nanoscience and Nanotechnology 6(3): 573-590).

g. Low or high-density lipoprotein (see, for example, Rudewicz-Kowalczyk, D. and Grabowska, I. 2021. “Detection of Low-density lipoprotein-Comparison of Electrochemical Immuno- and Aptasensor,” Sensors (Basel, Switzerland) 21(22): 7733; Chunta, S., Boonsriwong, W., Wattanasin, P., Naklua, W., and Lieberzeit, P. A. 2021. “Direct Assessment of Very Low-density lipoprotein by Mass Sensitive Sensor with Molecularly Imprinted Polymers,” Talanta 221:121549; Wu, H., Fang, F., Wang. C., Hong, X., Chen, D. and Huang, X. 2021. “Selective Molecular Recognition of Low Density Lipprotein Based on Beta-cyclodextrin-coated Electrochemical Biosensor,” Biosensors (Basel, Switzerland) 11(7) 216; Chunta, S., Suedee. R., Boonsriwong. W., and Lieberzeit, P. A. 2020. “Biomimetic Sensors Targeting Oxidized Low-density lipoprotein with Molecularly Imprinted Polymers,” Analytica Chimica Acta 1116:27-35; Narwal, V., Deswal, R., Batra, B., Kaira, V., Hooda. R., Sjarma, M., and Rana, J. S. 2019. “Cholesterol Biosensors: A Review,” Steroids 143:6-17; Chunta, S., Suedee. R., and Lieberzeit, P. A. 2018. “High-density lipoprotein Sensor Based on Molecularly Imprinted Polymer,” Analytical and Bioanalytical Chemistry 410(3): 875-883; Chunta, S., Suedee. R., and Lieberzeit. P. A. 2016. “Low-density lipoprotein Sensor Based on Molecularly Imprinted Polymer,” Analytical Chemistry 88(2): 1419-1425; Chang, T. Y., Chang, C. C., Ohgami, N., and Yamauchi, Y., 2006. “Cholesterol Sensing, Trafficking, and Esterification,” Annual Review of Cellular and Developmental Biology 22:129-157; Snellings. S., Fuller, J., Pitner, M., and Paul, D. W. 2003. “An Acoustic Wave Biosensor for Human Low-density Lipoprotein Particles: Construction of Selective Coatings,” Biosensors and Bioelectronics (Amsterdam, North Holland, Netherlands) 19(4): 353-363;

h. Coagulation, thrombus (see, for example, Chan, J., Michaelsen, K., Estergreen, J. K., Sabath, D. E., and Gollakota, S. 2022. “Micro-mechanical Blood Clot Testing Using Smartphones,” Online, Nature Communications (London, England) 13(1): 831; Jigar-Panchal. H., Kent, N. J., Know, A. J. S., and Harris, L. F. 2020. “Microfluidics in Haemostasis: A Review,” Online, Molecules 25(4): 833; Mohammadi, Aria, M., Erten, A., and Yalcin, O. 2019. “Technology Advancements in Blood Coagulation Measurements for Point-of-care Diagnostic Testing,” Online, Frontiers in Bioengineering and Biotechnology 7:395; Ren, L., Yu. K., and Tan, Y. 2019. “Applications and Advances of Magnetoelastic Sensors in Biomedical Engineering: A Review,” Online, Materials (Basel, Switzerland) 12(7): 1135; Li, Z., Wang. Y., Xue, X., McCracken, B., Ward, K., and Fu J. 2018. “Carbon Nanotube Strain Sensor Based Hemoretractometer for Blood Coagulation Testing,” Online, American Chemical Society Sensors 3(3): 670-676; Puckett, L. G., Lewis, J. K., Urbas, A. Cui, X., Gao, D., and Bachas. L. G. 2005. “Magnetoelastic Transducers for Monitoring Coagulation, Clot Inhibition, and Fibrinolysis,” Biosensors and Bioelectronics 20(9): 1737-1743; Puckett, L. G., Barrett, G., Kouzoudis. D., Grimes, C., and Bachas. L. G. 2003. “Monitoring Blood Coagulation with Magnetoelasic Sensors,” Biosensors and Bioelectronics 18(5-6): 675-681).

i. Stress, crisis, and cytokine detecting sensors. Extracorporeal embodiments already available, the advent of prosthetic disorder response systems will prompt the development of sensors to detect an incipient or emergent crisis such as a myocardial infarction, an integrated stress response, or a-cytokine hormonal regulator storm based upon the chemical analytes associated with these which are implantable or at least wearable. The specificity these can provide beyond that obtained with implantable pacemaker/cardioverter and cardiac resynchronization defibrillators warrants their development in implantable form.

Stress, crisis, and cytokine sensors suitable for use in a prosthetic disorder response system must be adapted to communicate with a probe in one or more biofluids such as sweat, saliva, or the blood as well as the control processor, and contrary to much current preference, not necessarily non-invasively but eventally, implantable (see, for example, Buskermolen, A. D., Lin, Y. T., van Smeden, L., van Haaften, R. B., Yan, J., Sergelen, K., de Jong. A. M., and Prins, M. W. J. 2022. “Continuous Biomarker Monitoring with Single Molecule Resolution by Measuring Free Particle Motion,” Online, Nature Communications (London, England) 13(1): 6052; Frimpong, A., Owusu, E. D. A., Amponsah, J. A., Obeng-Aboagye, E., van der Puije, W., and 3 others 2022. “Cytokines as Potential Biomarkers for Differential Diagnosis of Sepsis and Other Non-septic Disease Conditions,” Online, Frontiers in Cellular and Infection Microbiology 12:901433; He, J., Zhou, L., Huang, G., Shen, J., Chen, W., and 7 others 2022. “Enhanced Label-free Nanoplasmonic Cytokine Detection in SARS-COV-2 [acute respiratory syndrome coronavirus 2] Induced Inflammation Using Rationally Designed Peptide Aptamer,” Online, American Chemical Society Applied Materials and Interfaces 14(43): 48464-48475; Lubken, R. M., de Jong, A. M., and Prins, M. W. J. 2022. “Real-time Monitoring of Biomolecules: Dynamic Response Limits of Affinity-based Sensors,” Online, American Chemical Society Sensors 7(1): 286-295; Dutta, N., Lillehoj, P. B., Estrela, P. and Dutta, G. 2021. “Electrochemical Biosensors for Cytokine Profiling: Recent Advancements and Possibilities in the Near Future,” Online, Biosensors (Basel, Switzerland) 11(3): 94; Kartikasari, A. E. R., Huertas, C. S., Mitchell, A., and Piebanski, M. 2021. Op cit, above in subsection c. Tumor Cells, to Include Malignant and Circulating, and in subsection f, Inflammation; Khayamian, M. A., Parizi, M. S., Ghaderina, M., Abadijoo. H., Vanael. S., and 9 others 2021, “A Label-free Graphene-based Impedimetric Biosensor for Real-time Tracing of the Cytokine Storm in Blood Serum, Suitable for Screening COVID-19 Patients,” Online, Royal Society of Chemistry Advances 11(55): 34503-34515; Kotru, S., Klimuntowski, M., Ridha, H., Uddin, Z., Askhar, A. A., Singh, G. and Howlader, M. M. R. 2021. “Electrochemical Sensing: A Prognostic Tool in the Fight against COVID-19,” Online, Trends in Analytical Chemistry 136:116198; Liu, C., Chu, D., Kalantar-Zadeh, K., George, J., Young, H. A., and Liu, G. 2021. “Cytokines: From Clinical Significance to Quantification,” Online, Advanced Science (Weinheim, Baden-Würtemberg, Germany) 8(15): e2004433; Liu, G., Jiang. C., Lin. X., and Yang, Y. 2021. “Point-of-care Detection of Cytokines in Cytokine Storm Management and Beyond: Significance and Challenges,” Online, View (Beijing, China) 2(4): 202110003; Lohcharoenkal. W., Abbas, Z., and Rojanasakul, Y. 2021. “Advances in Nanotechnology-based Biosensing of Immunoregulatory Cytokines,” Online, Biosensors (Basel, Switzerland) 11(10): 364; Lubken, R. M., Bergkamp, M. H., de Jong, A. M., and Prins, M. W. J. 2021. “Sensing Methodology for the Rapid Monitoring of Biomolecules at Low Concentrations over Long Time Spans,” Online, American Chemical Society Sensors 6(12): 4471-4481; Song. Y., Zhao, J., Cai. T., Stephens, A., Su. S. H., and 7 others 2021. “Machine Learning-based Cytokine Microarray Digital Immunoassay Analysis,” Online, Biosensors and Bioelectronics 180:113088; Campuzano, S., Yanez-Sedeho, P., and Pingarron, J. M. 2020. “Revisiting Electrochemical Biosensing in the 21st Century Society for Inflammatory ytokine Involved in Autoimmune, Neurodegenerative, Cardiac, Viral, and Cancer Diseases,” Online, Sensors (Basel, Switzerland) 21(1): 189; Fajgenbaum, D. C. and June, C. H. 2020. “Cytokine Storm,” Online, New England Journal of Medicine 383(23): 2255-2273; Rodrigues, D., Barbosa, A. I., Rebelo, R., Kwon, I. K., Reis, R. L. and Correlo. V. M. 2020. Op cit., above in section 1a, entitled Background. General: Russell, S. M., Alba-Patino, A., Baron, E., Borges, M., Gonzalez-Freire, M., and de la Rica, R. 2020. Op cit., above under subsection f. entitled Inflammation; Wang, C., Cai, Y., MacLachlan, A., and Chen, P. 2020. “Novel Nanoplasmonic-structure-based Integrated Microfluidic Biosensors for Label-free in Situ Immune Functional Analysis: A Review of Recent Progress,” Online, Institute of Electrical and Electronics Engineers Nanotechnology Magazine 14(2): 46-C3; Brothers, M. C., DeBrosse, M., Grigsby, C. C., Naik, R. R., Hussain, S. M., Heikenfeld, J., and Kim, S. S. 2019. “Achievements and Challenges for Real-time Sensing of Analytes in Sweat within Wearable Platforms,” (American Chemical Society) Accounts of Chemical Research 52(2): 297-306; Singh, M., Truong, J., Reeves, W. B., Hahm, J. I. 2017. “Emerging Cytokine Biosensors with Optical Detection Modalities and Nanomaterial-enabled Signal Ehnancement,” Online, Sensors (Basel, Switzerland) 17(2): 428; Pakos-Zebrucka, K., Koryga, I., Mnich, K., Ljujic. M., Samali. A., and Gorman, A. M 2016. “The Integrated Stress Response,” European Molecular Biology Organization Reports 17(10): 1374-1395 (also cited above in section 1a, entitled Background. General); Chen, P., Huang, N. T., Chung, M. T., Cornell. T. T., and Kurabayashi, K. 2015. “Label-free Cytokine Micro- and Nano-biosensing towards Personalized Medicine of Systemic Inflammatory Disorders,” Online, Advanced Drug Delivery Reviews 95:90-103; Xiao, W., Mindrinos, M. N., Seok, J., Cuschieri, J. Cuenca A. G., and 35 others 2011. “Inflammation and Host Response to Injury Large-scale Collaborative Research Program. A Genomic Storm in Critically Injured Humans,” Online, Journal of Experimental Medicine 208(13): 2581-2590; additional references are cited above in subsection e, entitled Graft Organ Rejection Analytes).

j. Stroke or cerebral infarction is detectable by portable stroke detection devices from the change in blood flow to the brain (see, for example, Chennareddy, S., Kalagara, R., Smith, C., Matsoukas, S., Bhimani, A., and 7 others 2022. “Portable Stroke Detection Devices: A Systematic Scoping Review of Prehospital Applications,” Online, BioMed Central Emergency Medicine 22(1): 111; Lee, E. C., Ha. T. W., Lee, D. H., Hong, D. Y., Park, S. W., and 3 others 2022. “Utility of Exosomes in Ischemic and Hemorrhagic Stroke,” Online, MDPI [Multidisciplinary Digital Publishing Institute] International Journal of Molecular Sciences (Basel, Switzerland) 23(15): 8367; Patrick, L., Smith, W., and Keenan, K. J. 2021. “Large Vessel Occlusion Stroke Detection in the Prehospital Environment,” Online, Current Emergency and Hospital Medicine Reports,” 9(3): 64-72; Shahrestani. S., Wishart. D., Han, S. M. J., Stricland, B. A., Bakhsheshian, J., and 5 others 2021. “A Systematic Review of Next-generation Point-of-care Stroke Diagnostic Technologies,” Online, Journal of Neurosurgery Neurosurgical Focus 51(1): E11; Otero-Ortega, L., Laso-Garcia, F. Gomez-de Frutos, M., Fuentes, B., Diekhorst. L., Diez-Tejedor, E., and Gutierrez-Fernandez, M. 2019. “Role of Exosomes as a Treatment and Potential Biomarker for Stroke,” Translational Stroke Research 10(3): 241-249; Walsh, K. B., 2019. “Non-invasive Sensor Technology for Prehospital Stroke Diagnosis: Current Status and Future Directions,” International Journal of Stroke 14(6): 592-602; Chang, Y. H., Wu, K. C., Harn, H. J., Lin, S. Z., and Ding, D. C. 2018. “Exosomes and Stem Cells in Degenerative Disease Diagnosis and Therapy,” Online, Cell Transplantation 27(3): 349- 363).

k. Radiation (see, for example, Christensen, J. B., Togno, M., Bossin, L., Pakari. O. V., Safai, S., and Yukihara, E. G. 2022. “Improved Simultaneous LET [linear energy transfer] and Dose Measurements in Proton Therapy,” Online, Scientific Reports 12(1): 8262; de Freitas Nascimento. L., Leblans. P., van der Heyden, B., Akselrod, M., Goossens, J., and 3 others 2022. “Characterisation and Quenching Correction for an Al₂O₃:C [alumina:carbon] Optical Fibre Real Time System in Therapeutic Proton, Helium, and Carbon-charged Beams,” Online, MDPI [Multidisciplinary Digital Publishing Institute] Sensors (Basel, Switzerland) 22(23): 9178; Ba Sunbul, N. H., Zhang, W., Oraiqat. I., Litzenberg, D. W. Lam, K. L., and 8 others 2021. “A Simulation Study of Ionizing Radiation Acoustic Imaging (IRAI) as a Real-time Dosimetric Technique for Ultra-high-Dose Rate Radiotherapy (UHDR-RT),” Online, Medical Physics 48(10): 6137-6151; Setianegara, J., Mazur, T. R., Hao. Y., Yang, D., and Harold Li, H. 2021. “Development of a Storage Phosphor Imaging System for Proton Pencil Beam Spot Profile Determination,” Online, Medical Physics 48(9): 5459-5471; Setianegara, J., Mazur, T. R., Yang, D., and Li, H. H. 2021. “Dual-storage Phosphor Proton Therapy Dosimetry: Simultaneous Quantification of Dose and Linear Energy Transfer,” Online, Medical Physics 48(4): 1941-1955; Oraiqat. I., Zhang, W., Litsenberg, D., Lam, K., Ba Sunbul. N., and 5 others 2020. “An Ionizing Radiation Acoustic Imaging (IRAI) Technique for Real-time Dosimetric Measurements for FLASH [very brief ultra high dose external beam proton irradiation] Radiotherapy,” Online, Medical Physics 47(10): 5090-5101; Setianegara. J., Mazur, T. R., Maraghechi. B., Darafsheh, A., Yang, D., Zhao, T., and Li, H. H. 2020. “Quantitative Proton Radiation Therapy Dosimetry Using the Storage Phosphor Europium-doped Potassium Chloride,” Medical Physics 47(10): 5287-5300; Yada, R., Maenaka, K., Miyamoto, S., Okada, G., Sasakura, A., and 8 others 2020. “Real-time in Vivo Dosimetry System Based on an Optical Fiber-coupled Microsized Photostimulable Phosphor for Stereotactic Body Radiation Therapy,” Medical Physics 47(10): 5235-5249; Garty, G., Pujol-Canadell. M., and Brenner, D. J. 2019. “An Injectable Dosimeter for Small Animal Irradiations,” Online, Physics in Medicine and Biology 64(18): 18NT01; Matrosic. C., Culberson, W., Rosen, B., Madsen, E., Frank, G., and Bednarz, B. 2016. “Initial Characterization of a Gel Patch Dosimeter for in Vivo Dosimetry,” Online, Physics in Medicine and Biology 61(10): N240-N248; Han, Z., Driewer, J. P., Zheng. Y., Low, D. A., and Li, H. H. 2009. “Quantitative Megavoltage Radiation Therapy Dosimetry Using the Storage Phosphor KCl:Eu²⁺ [potassium chloride, europium doped],” Online, Medical Physics 36(8): 3748-3757; Black, R. D., Scarantino. C. W., Mann, G. G., Anscher, K. S., Ornitz, R. D., and Nelms, B. E. 2005. “An Analysis of an Implantable Dosimeter System for External Beam Therapy,” Online, International Journal of Radiation Oncology, Biology, Physics 63(1): 290-300; Scarantino, C. W., Ruslander, D. M., Rini, C. J., Mann, G. G., Nagle, H. T., and Black, R. D. 2004. “An Implantable Radiation Dosimeter for Use in External Beam Radiation Therapy,” Medical Physics 31(9): 2658-2671).

l. Imminent cardiac arrest. The use of implanted electronic heart failure monitoring and support devices to signal an impending terminal event is referenced below in section 2b(1)(a), entitled Sudden Compound Vascular Bypass, or Sudden Switched-organ Transplantation.

m. Urinary crystal (see, for example, Hennequin, C., Candelot. L., Ferroni A., Majoux, S. Beaudeux, J. L., and Nivet-Antoine, V. 2023. “Detection and Identification of Urinary Crystals Using an Automated Urine Particle Analyzer Coupled with a Digital Particle Imaging Device,” (in French with English abstract at Pubmed), Annales de Biologie Clinique (Paris, France) 81(1): 86-90; Hwang. C., Lee, W. J., Kim, S. D., Park S., and Kim, J. H. 2022. “Recent Advances in Biosensor Technologies for Point-of-care Urinalysis,” Online, MDPI [Multidisciplinary Digital Publishing Institute] Biosensors (Basel, Switzerland) 12(11): 1020). Here citation of pertinent research seeks to establish a basis potential or given for implementing the means delineated by a fully implanted automatic disorder response system.

n. Adverse reactions to drugs or combinations thereof, for example, unrelated to transplant rejection covered above under subsection e., entitled Graft Organ Rejection Analytes and Immunosensors. Some wearable sensors may be viewed as precursory to implanted embodiments. Adverse reactions to drugs comprehend diverse conditions such as stomach cramps, headache, dizziness, or impaired vision unrelated to any one particular kind of tissue reaction, physiological phenomenon, organ, organ system, or some combination of these, and therefore will never be detectable by any one type of sensor (see also, for example, Abdrakhimov, B., Kayewa, E., and Wang. Z. 2024. “Prediction of Acute Cardiac Rejection Based on Gene Expression Profiles,” Online, MDPI [Multidisciplinary Digital Publishing Institute] Journal of Personalized Medicine (Basel, Switzerland) 14(4): 410; Ding, M., Gao. T., Song. Y., Yi. L., Li, W., and 4 others 2024. “Nanoparticle-based T Cell Immunoimaging and Immunomodulatory for Diagnosing and Treating Transplant Rejection,” Online, Heliyon (London, England) 10(2):e24203; Jin, Y., Gao. P., Liang. L., Wang, Y., Li, J., and 4 others 2023. “Noninvasive Quantification of Granzyme B in Cardiac Allograft Rejection Using Targeted Ultrasound Imaging,” Online, Front Immunology (Lausanne, Switzerland) 14:1164183; Mac, Q. D., Mathews, D. V., Kahla, J. A., Stoffers. C. M., Delmas. O. M., and 3 others 2019. “Non-invasive Early Detection of Acute Transplant Rejection via Nanosensors of Granzyme B Activity,” Online, Nature Biomedical Engineering (London, England) 3(4): 281-291; Hurskainen, M., Ainasoja, O., and Lemstrom, K. B. 2021. “Failing Heart Transplants and Rejection—A Cellular Perspective,” Online, MDPI [Multidisciplinary Digital Publishing Institute] Journal of Cardiovascular Development and Disease (Basel, Switzerland) 8(12): 180; Welch, T. S., Mrsic, Z., and Mazimba, S. 2020. “Monitoring for Rejection,” in Bogar. L. and Stempien-Otero. A., eds, Contemporary Heart Transplantation, Springer International Publishing: Cham, Switzerland, pages 1-18).

When not reported by a preverbal infant, for example, the condition must be inferred on the basis of a detailed physical examination, and in the context of an implanted automatic disorder response system, detection by one or more of the type sensors enumerated above in this section, primarily those used to detect inflammation. Sensors that detect inflammation, for example, which may be indicative of an adverse drug reaction appear above in this section, subsection entitled f. Inflammation.

Relatively few papers address sensors to detect adverse reactions to drugs but rather adverse reactions to the sensors themselves. Accordingly, some references cite subject matter related to adverse drug reaction sensors as such (see, for example, Chalasani, S. H., Syed, J., Ramesh, M., Patil. V. and Pramod Kumar, T. M. 2023. “Artificial Intelligence in the Field of Pharmacy Practice: A Literature Review,” Online, Exploratory Research in Clinical and Social Pharmacy (New York, New York) 12:100346; Liu, Y., Li, J., Xiao, S., Liu, Y., Bai. M., and 3 others 2023. “Revolutionizing Precision Medicine: Exploring Wearable Sensors for Therapeutic Drug Monitoring and Personalized Therapy,” Online, MDPI [Multidisciplinary Digital Publishing Institute] Biosensors (Basel, Switzerland) 13(7): 726; Lu, T., Ji. S., Jin, W., Yang, Q., Luo, Q., and Ren, T. L. 2023. “Biocompatible and Long-term Monitoring Strategies of Wearable, Ingestible, and Implantable Biosensors: Reform the Next Generation Healthcare,” Online, MDPI [Multidisciplinary Digital Publishing Institute] Sensors (Basel, Switzerland) 23(6): 2991; Curulli, A. 2022. “Recent Advances in Electrochemical Sensing Strategies for Food Allergen Detection, Online, MDPI [Multidisciplinary Digital Publishing Institute] Biosensors (Basel, Switzerland) 12(7): 503; Edrees, H., Song, W., Syrowatka, A., Simona, A., Amato, M. G., and Bates, D. W. 2022. “Intelligent Telehealth in Pharmacovigilance: A Future Perspective,” Online, Drug Safety (Auckland, New Zealand) 45(5): 449-458; Salas, M., Petracek, J., Yalamanchili, P., Aimer, O., Kasthuril, D., and 3 others 2022. “The Use of Artificial Intelligence in Pharmacovigilance: A Systematic Review of the Literature,” Online, Pharmaceutical Medicine (Auckland, New Zealand) 36(5): 295-306; Bian, S., Zhu, B., Rong, G., and Sawan, M. 2021. “Towards Wearable and Implantable Continuous Drug Monitoring: A Review,” Online, Journal of Pharmaceutical Analysis (Xi'an, China) 11(1): 1-14; Basile, A. O., Yahi, A., and Tatonetti, N. P. 2019. “Artificial Intelligence for Drug Toxicity and Safety,” Online, Trends in Pharmacological Sciences (East London, England) 40(9): 624-635; Lenik, J. 2017. “Cyclodextrins Based Electrochemical Sensors for Biomedical and Pharmaceutical Analysis,” Current Medicinal Chemistry (Saif Zone, Sharjah, United Arab Emirates) 24(22): 2359-2391; Implanted devices other than radiation dosimeters may require radiation monitoring (see, for example, Chan, M. F., Young, C., Gelblum, D., Shi, C., Rincon, C., Hipp, E., Li, J., and Wang, D. 2021. “A Review and Analysis of Managing Commonly Seen Implanted Devices for Patients Undergoing Radiation Therapy,” Online, Advances in Radiation Oncology 6(4): 100732).

o. Sensors to determine the serum levels of hormones associated with thyroid and parathyroid function are addressed below in section 2b(1)(b)(b11), entitled Metered Compound Vascular Bypass, or Switched-organ Heterotopic Intact Allograft Thyroid and Parathyroid Transplantation.

### Microbial Intrusion

With an ability to break down the tissue of the host, a breach in the skin or the epithelial lining of an internal organ can create a portal through which bacteria can gain entry. Relatively common examples are the penetration through a break in the skin of bacteria such as the gram-negative Providencia rettgeri and group A beta hemolytic Streptococcus pyogenes and Staphylococcus aureus to cause erysipelas if not a deeper cellulitis (Dhar, D. 2006. “Bacterial Skin Infections,” Chapter 119, page 980, in The Merck Manual, Whitehouse Station, New Jersey: Merck Research Laboratories).

Another common example is the passage of pathogens by progressive enzymatic erosion and burrowing through tissue into neighboring internal structures. For example, severe constipation, most often in the elderly, that results in injury to the lining of the rectum can allow gastrointestinal bacteria such as Escherichia coli to pass into the detrusor resulting in infection of the urinary bladder (see, for example, Alighami, M. E., Barghash, M. M., Majaesic, E. Bhandari, V., and Houry, W. A. 2022. “Cellular Functions of the ClpP [tetradecameric serine protease] Protease Impacting Bacterial Virulence,” Online, Frontiers in Molecular Biosciences 9:1054408; Culp, E. and Wright, G. D. 2017. “Bacterial Proteases, Untapped Antimicrobial Drug Targets, Online, Journal of Antibiotics (Tokyo, Japan) 70(4): 366-377; Rao, M. B., Tanksale, A. M., Ghatge, M. S., and Deshpande, V. V. 1998. “Molecular and Biotechnological Aspects of Microbial Proteases,” Online, Microbiology and Molecular Biology Reviews 62(3): 597-635; McGregor, J. A., Lawellin, D., Franco-Buff, A., Todd, J. K., and Makowski, E. L. 1986. “Protease Production by Microorganisms Associated with Reproductive Tract Infection,” American Journal of Obstetrics and Gynecology (clinical section) 154(1): 109-114; Gill, C. O. and Penney, N. 1977. “Penetration of Bacteria into Meat,” Online, Applied and Environmental Microbiology 33(6): 1284-1286).

Once released thus, control over constipation, eradication of the bacteria, and healing of the rectal lining are essential to prevent reinfection. Here, where the site of reinfection is known, a basic side-entry jacket is positioned along an internal iliac artery to directly target an antibiotic such as ciprofloxacin or amoxicillin/clavulanate potassium into the bladder.

One or more sensors is positioned along an internal iliac vein to detect exotoxins, enzymes, and a leukocyte concentration diagnostic for infection. While direct venous drainage of the bladder is into the vesical venous plexus, no attempt is made to position a sensor along the veins of the plexus, which small in caliber and invested within other tissue would require needless and traumatizing dissection. Instead, system components are applied at a proximate level along the blood supply and drainage. Where the venous blood will provide the information needed and is readily accessed, to mount a sensor along the wall of the bladder is not preferred.

The closed pipeline containing and thus preventing exposure of uninfected tissue to the antimicrobial, a peak dose blood concentration (Levison, M. E. 2006. “Bacteria and Antibacterial Drugs,” Chapter 170, page 1409, in The Merck Manual, Whitehouse Station, New Jersey: Merck Research Laboratories) can be safely set much higher than if it were delivered by any alternative means of administration. Delivery thus also accomplishes isolation of the drug from drugs targeted elsewhere as well as nutrients in the general circulation.

Ductus side-entry jackets are positioned to encircle a tubular anatomical structure, or bodily conduit, to create a continuous passageway between the lumen of the conduit and the line leading to the jacket, the jacket itself, and the native lumen beyond the jacket. Side-entry jacket and nonjacketing connector mainlines and accessory channels can both be used to pass medication into the substrate ductus or volume of tissue. Accordingly, these devices can support movement inward or outward; that is, support either incurrent flow to deliver medication and provide a passageway to insert a cabled device, or excurrent flow to take a needle aspiration or a core or incisional biopsy tissue test sample, or to withdraw a cabled device, such as a fine fiber endoscope, intravascular ultrasound probe, or laser.

In an implanted automatic disorder response system, basic side-entry jackets, nonjacketing side-entry connectors, and vascular servovalves, to include flow diversion valves and servo chokevalves, can all be used in a coordinated manner to optimize circulation, for example. Such a system is intended to remain in place over the life of the patient. Means incorporated to allow for considerable growth, placement even in a small child should not require reentry to replace parts more than once.

Existing means for connecting a synthetic to an anatomical tube such as a ‘butterfly needle’ (scalp vein set, winged infusion set) an indwelling catheter cannot begin to meet a requirement for lifelong function. The connections essential to sustain such an implanted system cannot otherwise be produced. All are limited to short term use; neglecting to withdraw the devise within the recommended time will result in irritation, tissue damage infection, leakage, and/or obstruction.

The leading edge of the blood-conveying bloodline, or mainline connector, part number 6 in the drawing figures, is a stainless steel (inox), titanium, or tough plastic trepan trepan (trephine, tissue hole cutter, circular blade, punch) mainline end-piece, which is used with suction from an aspirator and a hooked retrieval tool to cut and retrieve the plug of tissue out of the vessel wall 2 will not deform when autoclaved, or steam sterilized (Dautzenberg, P., Volk, H. A., Heuls, N., Cieciora, L., Dohmen, K., and 3 others 2021. “The Effect of Steam Sterilization on Different 3D Printable Materials for Surgical Use in Veterinary Medicine,” Online, BioMed Central Veterinary Research 17(1): 389). An adverse allergic reaction to the nickel in stainless steel common, an alternative or coating material for the trepan is polyetheretherketone, or PEEK, which can be prepared with a razor-sharp cutting edge.

Labeled at its rear end, the trepan extends to its cutting edge at its front end. As a development of a ductus side-entry jacket, the placement of a vascular valve is the same as the description therefor provided in U.S. Pat. No. 11,759,186, entitled Ductus Side-entry Jackets and Prosthetic Disorder Response Systems, wherein the trepan is labeled as part number 6. Connected to a vacuum pump and placed against the outer surface of the ductus, the wall of the ductus is drawn past the razor sharp front edge to cut a circular hole (ostium, aperture) in the side of the ductus. The plug, of cylindrical core of tissue, is then removed by continued suction and if necessary, implements such as a hook-ended Amplatz or Technowood guidewire. The term ‘mainline’ applies when the fluid conducted is urine, as when drawn directly from a ureter through such a line into a collection bag.

The apparatus and methods described herein including heart transplantation and carotid endarterectomy, the question as to the inadvertent inducement of an arrhythmia or myocardial infarction as an immediate consequence of manipulating an artery-even an artery other than that triggering the event but a rarity using any means-warrants comment. Infrequent under any circumstances, the reduced level of handling required by the apparatus and methods prescribed herein should make the inducement of an arrhythmia intraoperatively even less common.

Such instances are attributable to prolongation of the QT interval, which in a carotid endarterectomy can arise through any of numerous and complex combinations of factors, to include medication, manipulation, especially of the carotid body, causing it to generate false output, and anesthesia, as well as the blood gas, electrolyte, endocrine, and neurological condition of the patient (see, for example, Ueda, Y., Asamoto, M., Kariya, T., Akai, T., Hoshina, K., and Uchida, K. 2022. “Ventricular Fibrillation during Carotid Endarterectomy and Bailout Stenting: A Case Report,” Online, JA [Journal of Anesthesia] Clinical Reports (Japanese Society of Anesthesiologists, Heidelberg, Germany. Springer Verlag) 8(1): 26; Shaik, F. A., Slotwiner, D. J., Gustafson, G. M., and Dai, X. 2020. “Intra-procedural Arrhythmia during Cardiac Catheterization: A Systematic Review of Literature,” Online, World Journal of Cardiology 12(6): 269-284; Hayasaka, K., Takigawa, M., Takahashi, A. Kuwahara, T., Okubo, K., and 14 others 2017. “A Case of Ventricular Fibrillation as a Consequence of Capecitabine-induced Secondary QT Prolongation: A Case Report,” Online, Journal of Cardiology Cases (Tokyo, Japan) 16(1): 26-29).

Significant adverse cardiac events up to a year following conventional carotid endarterectomy to include arrhythmia as that most common, cardiac arrest, myocardial infarction, and congestive heart failure taken collectively occur in about 3.5 percent of cases with stroke occurring in 4.7 percent (Suphathamwit, A., Leewatchararoongjaroen, C., Rujirachjun, P., Poopong, K., Leesakul, A, Junyavoraluk, A., and Ruangsetakit, C. 2022. “Incidence of Postoperative, Major, Adverse Cardiac Events in Patients Undergoing Carotid Endarterectomy: A Single-center, Retrospective Study,” Online, SAGE [SAra and GEorge, founders] Open Medicine 10:20503121211070367; Diuzhikov, A. A., Khripun, A. V., Malevannyi, M. V., Orlov, A. E., and Kulikovskikh, IaV. 2013. “Immediate and Remote Results of Using Carotid Endarterectomy and Stenting of Internal Carotid Arteries,” (in English and Russian) Angiologia i Sosudistaya Khirurgia (Angiology and Vascular Surgery) (Moscow, Russia) 19(2): 102-110. 93-100).

In addition to adjustment in the preprocedural physiological indicia indicative of patient susceptibility to atrial or ventricular fibrillation cited above, countermeasures before carotid endarterectomy include anticoagulation with stenting a postprocedural option (see, for example, Ueda, Y., Asamoto, M., Kariya, T., Akai, T., Hoshina, K., and Uchida, K. 2022, Op cit. preceding paragraph; Ugurlucan, M., Akay, H. T., Erdinc, I., Oztas, D. M., Conkbayir, C., and 4 others 2019. “Anticoagulation Strategy in Patients with Atrial Fibrillation after Carotid Endarterectomy,” Acta Chirurgica Belgica 119(4): 209-216; Engelter, S. and Lyrer, P. 2003. “Antiplatelet Therapy for Preventing Stroke and Other Vascular Events after Carotid Endarterectomy,” Online, Cochrane Database of Systematic Reviews 2003(3): CD001458).

Another adverse phenomenon which can arise responsive to the manipulation of an artery—even an artery other than that manipulated—is a vasospasm, which eventuality can prove very serious and even fatal (see, for example, Bai, Y., Bai, Y., Jiang, N., Chen, Q., and Guo, Z. 2022. “Perioperative Coronary Artery Spasm after Off-pump Coronary Artery Bypass Grafting in the Non-manipulated Coronary Artery,” Online, BioMed Central Cardiovascular Disorders 22(1): 166; Matta, A., Bouisset, F., Lhermusier, T., Campelo-Parada, F. Elbaz, M., Carrie, D., and Roncalli, J. 2020. “Coronary Artery Spasm: New Insights, Online, Journal of Interventional Cardiology 2020:5894586; Zhang, H., Zhang, W. J., Wu, Y. J., and Gao, R. L. 2016. “Recurrent Multivessel Coronary Artery Spasm Presented as Myocardial Infarction,” Online, Chinese Medical Journal (in English) 129(22): 2753-2756; Aikpokpo, N., Hill, S., and Sechtem, U. 2012. “Case Report: Acute Coronary Artery Spasm in a Patient in the Setting of Non-cardiac Surgery,” Clinical Research in Cardiology 101(8): 683-686; Baek, J. H., Han, S. S., and Lee, D. H. 2010. “Native Coronary Artery and Grafted Artery Spasm Just after Coronary Artery Bypass Grafting: A Case Report,” Online, Journal of Korean Medical Science 25(4): 641-643; Asano, T., Kobayashi, Y., Ohno, M. Nakayama, T., Kuroda, N., and Komuro, I. 2007. “Multivessel Coronary Artery Spasm Refractory to Intensive Medical Treatment,” Angiology 58(5): 636-639; Saito, H., Itoh, T., Itoh, M., Kanaya, Y., Suzuki, T., and Hiramori, K. 2007. “Simultaneous Multivessel Coronary Spasm Causing Acute Myocardial Infarction: A Case Report,” Angiology 58(1): 112-117). Based upon the potential for vasospasm even following off-pump coronary artery bypass grafting, this eventuality is not necessarily induced by ischemia.

By reducing the arterial connections to the aorta through an unified entry confluence conduit, direct aortocaval connection halves the number of arteries that must be handled and therewith the potential for vasospasm. Such pertains to the coronary arteries which are addressed in U.S. Pat. No. 11,759,186, entitled Ductus Side-entry Jackets and Prosthetic Disorder Response Systems, but uninvolved in the procedures addressed herein, to include carotid endarterectomy, switched-organ heart transplantation, and extracardiac correction of complete, or dextro-transposition, of the great arteries.

Because a switched-organ heart transplant eliminates any interruption in perfusion, not only is ischemia-reperfusion injury eliminated, likely extending the durability of the transplant considerably, but also minimized if not eliminated is the expectation of coronary artery spasm after myocardial revascularization (Mico-Carnero, M., Zaouali, M. A., Rojani-Alfonso, C., Maroto-Serrat, C., Ben Abdennebi, H., and Peralta, C. 2022, Op cit. above in this section; Buxton, A. E., Goldberg, S., Harken, A., Hirshfield, J. Jr., and Kastor, J. A. 1981. “Coronary-artery Spasm Immediately after Myocardial Revascularization: Recognition and Management,” New England Journal of Medicine 304(21): 1249-1253).

This eventuality is, however, relevant to the use of radial projection catheters and angioplasty-capable barrel-assemblies as delineated in U.S. Pat. No. 11,389,171, entitled Integrated System for the Infixion and Retrieval of Implants. In most applications, ductus side-entry jackets, vascular valves, servovalves, and nonjacketing side-entry connectors are not removed once the procedure has been completed but rather left in place to allow continued use of their accessory channels through the connected sidelines, or druglines, to implement automatic sensor input-driven follow-up drug treatment. The fully implanted automatic disorder response system can also incorporate diagnostic sensors, such as a tiny oximeter, signaling means for alerting the clinic to a cerebrovascular accident, diagnostic probes such as angioscopes, and electrical effectors such as neurostimulatory.

A drugline, or sideline, with entry into the end-device through an accessory channel, usually feeds into an ordinary ductus side-entry jacket, nonjacketing side-entry connector, diversion jacket, or vascular valve or servovalve, and remains usable whether the accompanying mainline is removed or left in place as a prosthesis, as might, for example, be opted for following an ischemia-free carotid endarterectomy where the postprocedural condition of the common, internal, and external carotids recommended this rather than depending upon the native anatomy. That is, in almost every instance except where it would encroach upon neighboring tissue, once a side-entry component has satisfied its primary purpose, it would be left in position to allow use of its accessory channels. One alternative is to replace the original component with one smaller and lighter in weight.

The potentially grave consequences posed by vasospasm-whether as a preexistent vasospastic angina, whether chronic or acute, much less midprocedural—has prompted the development of numerous drugs and emergency interventional measures for its prevention, abatement, and treatment. Vasospasm and its prevention are of central importance with respect to vascular valves and servovalves as these call for the handling of muscular and coronary arteries as addressed herein and in U.S. Pat. No. 11,759,186, entitled Ductus Side-entry Jackets and Prosthetic Disorder Response Systems.

Posing risk whether preexistent or provoked midprocedurally due to handling, promptness in the onset of action of a vasospasmic suppressant may be urgent with regard to the latter. For application thus, agents such as beta blockers, calcium channel blockers, and angiotensin-converting enzyme inhibitors if not already in use to treat an ongoing angina, are suitable for initiation ahead of and in preparation for surgery. While the onset of action of immediate-release nifedipine is less than 20 minutes, botulinum toxin and especially nitrates such as nitroglycerin are more suitable for midprocedural emergencies.

Administered orally, nitroglycerin, for example, becomes effective in 1 to 3 minutes and maximized at 5 minutes; if administered intravenously however, onset is immediate (see, for example, Khan, K. M., Patel, J. B., and Schaefer, T. J. 2022. “Nifedipine,” Online, Treasure Island, Florida: StatPearls Publishing Co.; Kim, K. H., Kernd, C. C., Adnan, G., and Schaller, D. J. 2022. “Nitroglycerin,” Online, Treasure Island, Florida: StatPearls Publishing Co.; Physicians' Desk Reference 2022. “Nitroglycerin-Drug Summary,” online at PDR.net).

However, while effective in reversing the pain if not the hypertension associated with coronary vasospasm, vasodilators cannot reverse cerebral vasospasm (Popp. L. M., Lowell, L. M., Ashburn, N. P., and Stopyra, J. P. 2021. “Adverse Events after Prehospital Nitroglycerin Administration in a Nationwide Registry Analysis,” Online, American Journal of Emergency Medicine 50:196-201; Bosson, N., Isakson, B., Morgan, J. A., Kaji, A. H., Uner. A., and 3 others 2019. “Safety and Effectiveness of Field Nitroglycerin in Patients with Suspected ST Elevation Myocardial Infarction,” Prehospital Emergency Care 23(5): 603-611; Engelberg, S., Singer, A. J., Moldashel, J., Sciammarella, J., Thode, H. C., and Henry, M. 2000. “Effects of Prehospital Nitroglycerin on Hemodynamics and Chest Pain Intensity,” Prehospital Emergency Care 4(4): 290-293; Levati, A., Solaini. C., and Boselli, L. 1998. “Prevention and Treatment of Vasospasm,” Journal of Neurosurgical Sciences (Turin, Italy) 42(1 Supplement 1): 27-31).

Numerous drugs and methods are used in the treatment of chronic and midprocedural vasospasm (see also, for example, Jesser. J. Potreck, A., Vollherbst. D., Seker, F., Chen, M., and 5 others 2023. “Effect of Intra-arterial Nimodipine on Iatrogenic Vasospasms during Endovascular Stroke Treatment-Angiographie Resolution and Infarct Growth in Follow-up Imaging,” Online, BioMed Central Neurology 23(5): 1; Swarup, S., Patibandla, S., and Grossman, S. A. 2023. “Coronary Artery Vasospasm,” Online, Treasure Island, Florida: StatPearls Publishing Co.; Das, J. M. and Zito, P. M. 2022. “Nimodipine,” Online, Treasure Island, Florida: StatPearls Publishing Co.; Hao, G., Chu, G., Pan, P., Han, Y., Ai, Y., Shi, Z., and Liang, G. 2022. “Clinical Effectiveness of Nimodipine for the Prevention of Poor Outcome after Aneurysmal Subarachnoid Hemorrhage: A Systematic Review and Meta-analysis,” Online, Frontiers in Neurology 13:982498; Yildiz, M., Ashokprabnu, N., Shewale, A. Pico, M., Henry, T. D., and Qesada, O. 2022. “Myocardial Infarction with Non-obstructive Coronary Arteries (MINOCA),” Online, Frontiers in Cardiovascular Medicine 9:1032436; Arps. K., Chakravartti, J., Hess, C. N., and Rao, S. V. 2021. “Ventricular Fibrillation due to Aortocoronary Vein Graft Spasm during Angiography: Case Report and Literature Review,” Online, Journal of the American College of Cardiology 3(3): 388-391; Goldberg, S. H., Akoon, A., Kirchner, H. L., and Deegan, J. 2021. “The Effects of Botulinum Toxin A on Pain in Ischemic Vasospasm,” Online, Journal of Hand Surgery American 46(6): 513.e1-513.e12; Jewulski, J., Khanal, S., and Dahal, K. 2021. “Coronary Vasospasm: A Narrative Review,” Online, World Journal of Cardiology 13(9): 456-463; Liu, W., Shen, J., Li, Y., Wu, J., Luo, X., and 6 others 2021. “Pyroptosis Inhibition Improves the Symptom of Acute Myocardial Infarction,” Online, Cell Death and Disease 12(10): 852; Savage, M. P. and Moe. T. G. 2021. “Coronary Vasospasm: Not Gone but Often Forgotten,” Online, Journal of the American College of Cardiology Case Reports 3(3): 397-399; Sheth, M. A., Widmer, R. J., and Dandapantula, H. K. 2021. “Pathobiology and Evolving Therapies of Coronary Artery Vasospasm,” Online, Baylor University Medical Center Proceedings 34(3): 352-360; Kook, H., Hong, S., Yang, K. S., Lee, S., Kim, J. S., and Park, C. G. 2020. “Comparison of Nebivolol versus Diltiazem in Improving Coronary Artery Spasm and Quality of Life in Patients with Hypertension and Vasospastic Angina: A Prospective, Randomized, Double Blind Pilot Study,” Online, PLoS [Public Library of Science] One 15(9): e0239039; Beijk, M. A., Vlastra. W. V., Delewi. R., van de Hoef. T. P. Boekholdt, S. M., Sjauw, K D., and Piek, J. J. 2019. “Myocardial Infarction with Non-obstructive Coronary Arteries: A Focus on Vasospastic Angina,” Online, Netherlands Heart Journal 27(5): 237-245; Choo, E. H., Chang, K., Lee, K. Y., Lee, D., Kim, J. D., and 7 others 2019. “Prognosis and Predictors of Mortality in Patients Suffering Myocardial Infarction with Non-obstructive Coronary Arteries,” Online, Journal of the American Heart Association (Oxford, England) 8(14): e011990; Han, S. H., Lee, K. Y., Her, S. H., Ahn, Y., Park, K. H., and 11 others 2019. “Impact of Multi-vessel Vasospastic Angina on Cardiovascular Outcome,” Atherosclerosis 281:107-113; Tandon, V., Moseback, C. M., Kumar, M., and Joshi, S. 2019. “Refractory Vasospastic Angina: When Typical Medications Don't Work,” Online, Cureus (Palo Alto, California) 11(2): e4134; Bertic, M., Chue, C. D., Virani, S., Davis, M. K., Ignaszewski, A. and Sedlak. T. 2018. “Coronary Vasospasm following Heart Transplantation: Rapid Progression to Aggressive Cardiac Allograft Vasculopathy,” Canadian Journal of Cardiology 34(12): 1687.e9-1687.e11; Lim, Y., Singh, D., Loh, P. H., and Poh, K. K. 2018. “Multivessel Coronary Artery Spasm in Pericarditis,” Online, Singapore Medical Journal 59(11): 611-613; Tallarico. R. T., Pizzi. M. A., and Freeman, W. D. 2018. “Investigational Drugs for Vasospasm after Subarachnoid Hemorrhage,” Expert Opinion on Investigational Drugs 27(4): 313-324; Teragawa, H., Oshita, C., and Ueda. T. 2018. “Coronary Spasm: It's Common, but It's Still Unsolved,” Online, World Journal of Cardiology 10(11): 201-209; Slavich, M. and Patel, R. S. 2016. “Coronary Artery Spasm: Current Knowledge and Residual Uncertainties,” Online, International Journal of Cardiology. Heart and Vasculature 10:47-53; Kiser, T. H. 2013. “Pharmacologic Options for Prevention and Management of Cerebral Vasospasm in Aneurysmal Subarachnoid Hemorrhage,” Online, Hospital Pharmacy 48(5 Supplement): S2-S9; Mascia. L. and Del Sorbo, L. 2009. “Diagnosis and Management of Vasospasm,” Online, F1000 [faculty of 1000] Medicine Reports 1:33; van Beek, A. L., Lim, P. K., Gear, A. J. L., and Pritzker, M. R. 2007. “Management of Vasospastic Disorders with Botulinum Toxin A,” Plastic and Reconstructive Surgery 119(1): 217-226; (see also section 2b(1)(b)(b5) below, entitled Heterotopic Double Heart Metered Compound Vascular Bypass, or Switched-organ Transplantation and the Use of Unified Entry Arterial and Venous Confluence Conduits to Avoid Disuse Atrophy when the Graft Organ is Positioned in the Lower Abdomen by Direct Aortocaval Connection; see also vasodilators in section 2b(2)(h)(h3) below, entitled The ‘Tuned’ Shunt in the Relief of Pulmonary Arterial Hypertension).

Citing the extracranial carotids as exemplary of comorbid vascular disease, when these vessels are observed to not only have become atherosed (atheromatous, atherosclerosed), but whether aneurysmal, dissected, or flow-mediated vasodilated due to Marfan's syndrome, for example, or malacotic due to a connective tissue disorder, malignant invasion, trauma, or otherwise additionally diseased in such manner as to have become structurally weakened, and/or extensively deformed, or subject to recurrent stenosis, so that—especially following earlier stenting or endarterectomy—a stent-graft or saphenous or femoral vein interposition graft are felt inadequate, the synthetic common carotid with branches bypass is applied ab initio as a prosthesis and the native carotid with branches removed (see, for example, Benson, J. C. and Brinjikji, W. 2019. “The Chalice Sign: Characteristic Morphology of the Cervical Carotid Bifurcation in Patients with Loeys-Dietz Syndrome,” Clinical Neuroradiology September 24; Fatima, J., Federico. V. P., Scali, S. T., Feezor, R. J., Berceli, S. A., and 5 others 2019. “Management of Patch Infections after Carotid Endarterectomy and Utility of Femoral Vein Interposition Bypass Graft,” Journal of Vascular Surgery (New York, New York) 69(6): 1815-1823.e1; Lopes, A., Gomes, M. L., Sobrinho, G., and Pedro, L. M. 2019. “Surgical Treatment of Post-carotid Endarterectomy Carotid Pseudoaneurysm,” European Journal of Vascular and Endovascular Surgery Short Reports 46:12-13; Şimşek, B., Yüksel. V., Hüseyin, S., Güçlü, O., and Canbaz, S. 2019. “Ruptured Internal Carotid Artery Pseudoaneurysm 10 Years after Cervical Spine Surgery,” Vascular Specialist International (Seoul, South Korea) 35(2): 111-113: Singh, A. A., Velineni. R., Varty, K., and Gohel, M. S. 2019. “Bilateral Extracranial Internal Carotid Artery Aneurysms in a Patient with Marfan Syndrome: Case Report and Review of the Literature,” European Journal of Vascular and Endovascular Surgery Short Reports 45:14-16; Wangqin, R., Krafft. P. R., Piper, K., Kumar, J., Xu, K., Mokin, M., and Ren, Z. 2019. “Management of De Novo Carotid Stenosis and Postintervention Restenosis-Carotid Endarterectomy versus Carotid Artery Stenting—a Review of Literature,” Translational Stroke Research 10(5): 460-474; Welby, J. P., Kim, S. T., Carr. C. M., Lehman, V. T., Rydberg, C. H., and 4 others 2019. “Carotid Artery Tortuosity is Associated with Connective Tissue Diseases,” American Journal of Neuroradiology 40(10): 1738-1743; Basu, D. 2018. “Management of the Neck with Carotid Artery Involvement,” Online at emedicine.medscape.com/article/850738-overview; Lomeli, O., Perez- Torres, I., Marquez, R., Criales, S., Mejia, A. M., and 3 others 2018. “The Evaluation of Flow-mediated Vasodilation in the Brachial Artery Correlates with Endothelial Dysfunction Evaluated by Nitric Oxide Synthase Metabolites in Marfan Syndrome Patients,” Online, Frontiers in Physiology 9:965; Texakalidis, P., Giannopoulos, S., Charisis, N., Giannopoulos, S., Karasavvidis. T., Koullias, G., and Jabbour, P. 2018. “A Meta-analysis of Randomized Trials Comparing Bovine Pericardium and Other Patch Materials for Carotid Endarterectomy,” Journal of Vascular Surgery (New York, New York) 68(4): 1241-1256.e1; Varetto, G., Trevisan, A., Barile, G., Gibello, L., Spalla, F., and 3 others 2018. “Carotid Pseudoaneurysm after Eversion Endarterectomy: A Case Report and Review of the Literature,” Vascular and Endovascular Surgery 52(4): 309-312; Kim, S. T., Brinjikji, W., Lanzino, G., and Kallmes, D. F. 2016. “Neurovascular Manifestations of Connective-tissue Diseases: A Review,” Interventional Neuroradiology 22(6): 624-637; Ni. L., Pu, Z., Zeng. R., Zhang, R., Zheng, Y. H., Ye. W., and Liu, C. W. 2016. “Endovascular Stenting for Extracranial Carotid Artery Aneurysms: Experiences and Mid-term Results,” Medicine (Baltimore, Maryland) 95(46): e5442; Song, G., Jiao. L., Chen. Y., Wang. Y., Ma. Y., and Ling, F. 2015. “Management of Extracranial Carotid Artery Aneurysm: A Report of Four Cases,” Experimental and Therapeutic Medicine (Athens, Greece) 10(3): 1029-1032; Yang. W. I., Shim, C. Y., Cho, I. J., Chang, H. J., Choi. D., and 4 others 2010. “Dyssynchronous Systolic Expansion of Carotid Artery in Patients with Marfan Syndrome,” Journal of the American Society of Echocardiography 23(12): 1310-1316; Schonholz, C., Krajcer, Z., Carlos Parodi, J., Mendaro, E., Hannegan, C., and 4 others 2006. “Stent-graft Treatment of Pseudoaneurysms and Arteriovenous Fistulae in the Carotid Artery,” Vascular (London, England) 14(3): 123-129; Patel, J. V., Rossbach, M. M., Cleveland, T. J., Gaines, P. A., and Beard, J. D. 2002. “Endovascular Stent-graft Repair of Traumatic Carotid Artery Pseudoaneurysm,” Clinical Radiology 57(4): 308-311; Coumans, J. V., Watson, V. E., Picken, C. A., and McGrail, K. M. 1999. “Saphenous Vein Interposition Graft for Recurrent Carotid Stenosis after Prior Endarterectomy and Stent Placement. Case Report,” Journal of Neurosurgery 90(3): 567-570).

The terms ‘bypass,’ ‘compound vascular bypass,’ and ‘switch’ transplantation’ denote the transfer of circulation from the donor, or graft, directly to the recipient organ while still in the donor. Where matching and tolerance induction have been accomplished, this transfer can be sudden, with removal of the innate organ and its replacement with the graft organ in immediate succession. While the transfer requires that donor and recipient be positioned beside one another, preliminary match testing can be remote. Only in the most exigent circumstances would a sudden compound vascular bypass, or sudden switched-organ transplantation be performed without such preparation. Because such procedures require the compresence of both donor and recipient, smaller centers without heliports or quick access to a regional airport would arrange for such in advance.

The added advantage of continuous monitoring and response of a totally implanted system removes a central detraction of organ transplantation in that the patient is saved, but the constant vigilance and the need to be ever vigilant to administer immunosuppressive and other drugs virtually replaces one disease for another. The implanted system not only releases medication as prescribed, but except for systemic release when unavoidable, directly targets it, bypassing and thus avoiding the inducement of adverse side effects in nontargeted tissue. The automatic release of drugs is especially beneficial in the very young and very old who often fail to adhere to the prescribed drug regimen.

The reason that life support is currently stopped following the death of a prospective organ donor is precisely the lack of a means for directly and seamlessly transferring the graft organ from the circulatory system of the donor into the circulatory system of the recipient with no interruption in the delivery of oxygenated blood, and in so doing, avert the integrated stress response, cytokine storm-generating havoc, and ‘catecholamine surge, or ‘autonomic storm’ associated with death, which in a significant proportion of all heart transplants, is likely to prove a key factor in the eventual failure of the graft organ (see, for example, Beckman, E. J. 2019, Op cit. in this section). The removal of tissue, much less an intact organ, with hormonal and neurological connections that eventually extend throughout the body, to suddenly sever it from its unique situation within this milieu, represents a stunning insult that induces a long memory.

Nowhere is the benefit of an anoxia-free heart transplant more propitious than at the outset of life, in a neonate with a heart which due to developmental factors grew too malformed in utero to allow normal circulation despite the best of reconstructions or a conventional transplant, whether alone or with the addition of a mechanical assist device. Every tissue and organ would be spared the secondary maldevelopment due to hypoxia that would continue to the end of a sick life shortened for that reason in itself as well as the stress of retransplantation and other surgical procedures.

Such contemplates that a new heart without the experience and repercussions of its host having died is more likely to survive (see, for example, Delaura, I. F. Gao, Q., Anwar I. J. Abraham, N., Kahan. R., Hartwig. M. G., and Barbas, S. 2022. “Complement-targeting Therapeutics for Ischemia-reperfusion Injury in Transplantation and the Potential for Ex Vivo Delivery,” Online, Frontiers in Immunology (Lausanne, Switzerland) 13:1000172; Atkinson, C., Floerchinger, B., Qiao, F., Casey, S., Williamson, T., and 6 others 2013. “Donor Brain Death Exacerbates Complement-dependent Ischemia/Reperfusion Injury in Transplanted Hearts,” Circulation 127(12): 1290-1299; Lei. B., Sleiman, M. M., Cheng, Q., Tu Z. Zhu, P., and 6 others 2021. “In Situ Pre-treatment of Vascularized Composite Allografts with a Targeted Complement Inhibitor Protects against Brain Death and Ischemia Reperfusion Induced Injuries,” Online, Frontiers in Immunology (Lausanne, Switzerland) 12:630581).

Whether sudden or metered, an advantage of bypass, or sudden switched-organ, heart transplantation is that neither donor nor recipient hearts are incised; connection is of the blood supply and drainage, anastomosis of nerve stumps when considered warranted accomplished only once the new heart has been implanted. Because neither heart is incised, should for any reason the surgical team or the automatic disorder control system detect a need to terminate the procedure, both hearts will have remained in their native positions at the level of function they were prior to initiation of the procedure, the donor organ remaining available for transplantation in another patient. Donor and patient having been immunologically matched and tolerance induction therapy having been accomplished, the odds that an adverse reaction sufficiently intense to warrant terminating the procedure is unlikely.

During metered compound vascular bypass, or sudden switched-organ transplantation, the system modulates the rate of organ transfer by advancement of the vascular side-entry servovalve diversion chutes in accordance with the moment-by-moment intensity of any immunological reaction detected. At any moment, the chutes can be increased or decreased in rate of advancement or retraction, paused, or reversed as necessary, appropriate responsive medication in accordance with the prescription-program automatically targeted to the site or sites of the reaction whether local, that is, associated with the graft organ, or remote.

Preliminary blood type and human leukocyte antigen cross-matching of donor and recipient and the administration of tolerance-inducing medication will with rare exceptions have precluded hyperacute rejection so early and/or pronounced as to be detected during the processing of a metered transplant; intentionally extending the processing time over a period of hours in the expectation of intercepting a hyperacute rejection is never justified-such might rarely appear, but not for hours after the transfer has been completed.

Acute rejection does not appear for days if not weeks, and is initially detected and responded to by the implanted automatic response system in its continuous postprocedural maintenance, not its transfer function. This is true in conventional heart transplantation where the graft organ had not been removed until after the donor died, blood flow is not continuous during transfer from the donor to the recipient, and there is no gradual blending and replacement of the graft blood, which should materially reduce the odds for a bad outcome compared to a conventional transplant.

While improbable, midprocedural detection by the implanted system peptide cytokine sensors in proximate lymph nodes of T cell-secreted interleukin-2 as might suggest eventual acute rejection is not necessarily used to terminate the procedure. Rather—a better matched heart not forthcoming—this is noted in the record to signal the need to adjust the prescription-program accordingly to dispense immunosuppressive medication beyond the ordinary in response to this finding. For heart transplantation, the choice of sensors and medication programmed are keyed to detect and suppress the eventual development of cardiac allograft vasculopathy.

When anastomosed, donor and recipient stumps are facilitated to heal into a functional bond by the direct delivery of immunosuppressive and anti-inflammatory medication to the anastomoses through upstream side-entry jackets as shown in FIG. 22. In most instances, these will be the same upstream side-entry jackets that had been used to transfer between donor and recipient circulatory systems so that no additional jackets will be needed. In marked contrast with all conventional forms of heart transplantation, that neither heart is incised means that should a significant adverse reaction occur, both hearts will have been ruined with the option to withdraw having been eliminated.

Solenoid-driven valves accomplish this suddenly, while servovalves allow the gradual transition from the donor to the recipient, which affords an interval during which immune tolerogenic induction can be accomplished. In the present context, the terms vascular valve, diversion valve, valve jacket, and diversion jacket are synonymous and can refer to either solenoid or servomotor driven valves. Essentially, as opposed to metered, or gradual transfer of the graft organ from the circulatory system of the donor into that of the recipient, sudden compound vascular bypass, or sudden switched-organ transplantation responds to urgency in end-stage disease with death imminent as to require that the affected organ be replaced as soon as possible. Otherwise, whenever possible, thorough periprocedural immune matching, preparation, and induction tolerance are prescribed.

The term ‘metered switch’ transplantation denotes a tightly controlled seamless transfer of circulation from the recipient to the donor, or graft, organ at a rate directly responsive to the detection of rejection analytes by implant sensors. Demanding the continuous and immediate detection and coordination of multiple data streams and immediate response to any adverse indicants, this process is entrusted to a master controller to execute automatically in accordance with a prescription-program. Such programs are prepared and archived to administer a type procedure, such as a heart, kidney, or heart and kidney transplant, allowing for the variables encountered with each.

An emergency condition that justifies use of the sudden rather than the metered switch technique is encountered when the patient arrives without having been stabilized, or even if having been an inpatient, experiences a potentially fatal cardiac accident, for example, which if conventionally treated would still leave the patient with end-stage heart failure intractable to medical treatment and posing the risk of death. Barring such an exigent circumstance, metered compound vascular bypass, or sudden switched-organ transplantation is to be preferred as minimizing the immune impact and trauma of the corresponding conventional procedure. This is especially true of the heart with its multiple points for the in- and outflow of blood. Due to the abruptness of sudden bypass, or sudden switched-organ, transplantation, confirmation of compatibility before initiating the process is crucial.

When urgency does not allow sufficient time to adequately evaluate compatibility, sudden compound vascular bypass, or sudden switched-organ transplantation can be preceded by supplementary manual infusion of tolerance-inducing, or cross chimerizing, cells and drugs. However, the valves used to perform a metered compound vascular bypass, or sudden switched-organ transplantation instantly adaptable to the reaction to this infusi on as reported to the implanted disorder response system controller by implanted sensors, initiation of the process by metered switching is preferable as allowing immediate cessation, or ‘bailout,’ or if the reaction is nonacute, temporary ‘watchful waiting’ until the adverse reaction subsides.

Homozygous, or identical twins, or in experimental work, cloned replicate specimens of the source of index specimen, can be sudden compound vascular bypass, or sudden switched-organ transplanted without concern. However, the number of variables involved in an orthotopic or a heterotopic double compound vascular bypass, or sudden switched-organ heart transplant prohibits a reduction to and straightforward delineation of a simple procedure. In both single and double heart transplants, the detailed condition of the heart or hearts, comorbidity, organ system interconnections, electrolyte disturbances, available drugs, and alternative means of therapy must all be taken into account.

To this, double heart transplantation adds the need to coordinate the relative ejection fractions, or absolute stroke volume, of either heart as well as their timing. Following a metered switch heart transplant, the vascular servovalves used to connect all of the bloodlines are retained in place where these remain capable of automatic sensor-driven adjustments in flow rate and direct drug delivery through their accessory channels. While a bypass orthotopic heart transplant allows donor and recipient stumps to be anastomosed, a switch bypass double heart transplant is exceptional in not allowing sufficient proximity between the hearts to bring the stumps together. FIG. 22 shows the more usual situation where anastomosis is possible.

That anastomosed, the implanted controller can switch between the bypass and native route in coordination with the release of medication directly pipeline-targeted to the treatment site is a significant factor in facilitating healing. Both single and double heart bypass, or sudden switched-organ, transplantation can do much to alleviate the shortage in hearts available for transplantation, which due to the current means for preservation, are invariably impaired upon placement and likely to fail due to cardiac allograft vasculopathy, for example. Current methods guarantee that the distress caused by a defective heart will not have been obliterated at the outset but instead persist throughout a much shortened and miserable life, wherein a mind that might otherwise have been capable of high accomplishment is instead damaged and likely besought with anxiety over an early death.

In children born with materially defective if not unsurvivably malformed hearts, only replacement of the native heart with one or two hearts capable of normal, pulsatile, normovolemic (euvolemic), or non-Fontan circulation is able to overcome the deterioration in all other organs that ensues following even an adept repair. Mechanical pumps able to simulate normal circulation have posed problems of clotting and a lack of dependability. Together, bypass transplantation and continuous monitoring and responsive treatment by an implanted automatic system should overcome the need for retransplantation. Double heart transplantation is also useful as a bridging strategy that provides normal circulation pending the availability of a good heart.

End stage heart failure in the elderly is usually controllable with drugs and mechanical assist devices, but this is not so in a prenate or neonate with a heart that is unsurvivably malformed and will continue to prevent normal development throughout the body. Not a bridging strategy to transplantation as is often the object in placing a total artificial heart or ventricular assist device, a double heart transplant provides pulsatile circulation with the placement of a second heart to cooperate with that native in a relation of mutual support intended to serve to longer than a conventional transplant if not to the end of life. Sensors incorporated into the vascular servovalves or positioned separately continuously monitor the hearts so that upon detection of a dysrhythmia, or of one of more analytes indicative of rejection, for example, the implanted controller responds in accordance with its prescription-program.

The response can consist of the direct targeting drugs to the site, adjustments to the vascular servovalves, and/or the delivery through prepositioned electrostimulators of synchronization discharges to ameliorate if not reverse the problem. These measures, immediately executed automatically while the patient is ambulatory stand to avert more serious consequences that would ensue were attention deferred pending arrival at a clinic where to perform a diagnosis would itself take time. Continuous protection supported by coordinated means for immediate response medicinal and electrical to an exigency should extend the durability of the graft organ well beyond that associated with conventional transplantation. Mechanical assist devices are not only characterized by limited life but an expense that makes these prohibitive in much of the world.

Any means for imparting normal circulation which depends in whole or part upon mechanical components must answer to the reliability of the components employed. Sudden and metered switch heart transplantation is made possible by vascular valves. These are less complex than are alternative devices such as ventricular assist devices and total artificial hearts, which do not provide normal blood flow in any event, making these properly unacceptable for use in children. By contrast, the bypasses used in switch transplantation can back up anastomoses of the donor to the recipient vessels and the implanted automatic disorder control system can switch between the direct and bypass pathways in response to sensor inputs that indicate a malfunction or as specified in the prescription-program of the implanted controller.

This alteration can be used not only to occasionally relieve stress during healing, but the valves used can be left in place to directly pipeline-target drugs to the locus of inflammation or the appearance of a lesion, for example, wherever it arises. Vascular valves can be made to a level of reliability which exceeds that essential. In a bypass heart transplant, when the donor organ is removed, the highly elastic bloodlines, or mainlines, relieved of the pull exerted by traversing the distance between recipient and donor organs, contract in length, but if still too long, will ‘bunch up’ as to remain nonimplantable, so that a mechanism for replacing the long for a short line, the bloodline switch, implanted with the shorter line, shown in FIGS. 32A thru 32G, 33A, 33B, and 35B is used.

FIGS. 32A thru 32C show doubled bloodlines with adult nonvaulted bloodline-switches that allow the longer line to be removed, leaving a shorter bloodline to which blood flow had been switched to continue oxygenation of a donor graft organ with no interruption in the flow of blood. This sequence can be reversed to accommodate growth over a longer period than could highly stretchable accordioned or convoluted tubing. This solution is more decisive in allowing the excess length or slack in a longer line to be removed without the need to tuck it in amid surrounding tissue and leaving it there after having closed, possibly inducing an annoying sensation of a foreign object inside the body.

This problem becomes more prominent the more are the lines to the vessels of the same organ, as seen in FIG. 16, this number nine in a bypass, or sudden switched-organ orthotopic or bypass, or sudden switched-organ heterotopic double heart transplant. One of the several benefits in the use of confluence conduits such as shown in FIG. 35C is to visually simplify the operative field. In recipient end stage heart failure where positive inotropes fail to produce sufficient force to propel the blood from the recipient to the donor, a pump in the larger of the two lines overcomes this problem.

To prevent return flow by circulation of half the line outflow through the blood confluence chamber, part number 178 in FIG. 32G, and out into the return line so that only half the flow intended continues forward, the receiving line-switch also requires a line inflow stopper 175 to close off the entry into the other line. As can the vascular valves shown in FIGS. 22C thru 22E, bloodline switches can also be used to automatically switch between passage through an anastomosis and its bypass and/or the reverse. Such limited application contemplates that the line with the anastomosis is organic.

Under the control of an automatic disorder response system, receipt in a simple system by the microcontroller, or in a more complex or hierarchical system, the master control microprocessor, of sensor-signaled biomarkers indicating any of a number of possible problems and the choice of solutions, or a specific problem and the solution indicated, the controller signals the drug reservoir to pipe the agent directly to the site. This assumes that the number of such complications is known and limited so that to provide the necessary drug reservoirs is practicable.

Following a bypass transplant, the automatic disorder response system components which had administered the transplant are left in place to apply follow-up therapy. The components of the implant system having been devised for permanent implantation, system removal is seldom preferred. Especially in an elderly patient with impaired immune function, besought by multiple morbidities or one presenting a condition subject to recrudesce or later metastasis, the servovalves and druglines used to perform the transplant are left in. When based upon the findings of the physical examination and/or in response to sensor data fed to the automatic response system controller, the treatment site or medication must be changed, the system components and prescription-program programmed to initiate an optimizing routine are not explanted but redirected.

Most predictable eventualities such as the appearance of a dysrhythmia following a bypass double heart transplant, are accounted for in the initial placement of the system. While the fluid line delivering drugs into the accessory channels of the jackets or valves are separable from the accessory channels per se as providing one continuous passageway from the small flat drug reservoir implanted subdermally, or subcutaneously, in the pectoral region, to the native lumen, the two are usually referred to collectively as accessory channels, the distinction between the lines and passageway through the jacket or valve taken for granted. The retention of a servovalve with druglines at the connections of the bloodlines allows control over the flow past each. Before and after views of the disposition of the servovalves before harvesting and implantation of a graft organ, here the left lung, are shown in FIGS. 22A and 22B.

Much the same as switching between the bypass and native ductus following a carotid endarterectomy as shown in FIGS. 25A and 25B where transection and anastomosis of supply and drainage vessels are uninvolved, following a transplant such as shown in FIGS. 22A and 22B, where transection and anastomosis of supply and drainage vessels are involved, switching thus affords additional advantages. Accessory channels to deliver immunosuppressants to the severed ends of the donor and recipient vessels can also continue to induce immune tolerance until anastomosis is accomplished endoscopically through ‘keyhole’ incisions.

Exposed to the systemic medication, the intraluminal sides of anastomoses 46 and 46′ are already medicated. Accessory channels 154 and 154′ on the outside of the vessels can, as shown in FIG. 22B, drip drugs such as immunosuppressive, anti-inflammatory, and antimicrobial to wet the cut ends of the anastomoses on the outside of the vessels. Retention of the bypass thereafter allows switching between the bypass and native ductus. Switching thus can be accomplished automatically by the implanted controller in response to sensor feedback indicative of rejection requiring immunosuppression or disease requiring the appropriate drugs.

FIG. 22B shows that the valves straddle the anastomoses at a small distance, so that when donor and recipient stumps are sutured, the valves are positioned to deliver medication directly to the site in support of healing. More generally, because direct pipeline-targeting eliminates the exposure of nontargeted tissue to drugs harmful to unaffected tissue allowing that dispersed throughout the circulation to be reduced, the direct pipeline-targeting of drugs such as immunosuppressive or chemotherapeutic to a site of disease eliminates such exposure as a cause for increased susceptibility to infection and malignancy. While certain agents are inherently drawn to a certain target such as iodine to the thyroid, uptake thus amounts to 20 to 33 percent of the total, the remainder of 67 to 80 percent continuing to be exposed to nontargeted tissue.

As seen in FIGS. 22A and 22B showing the doubling of valves following placement of the graft lung in the recipient, upon completion of a switch transplantation, the valves on the stumps of the graft organ and the recipient organ since removed will be brought into proximity to either side of the stump junctions where these would normally be anastomosed in accordance with the distance from the transection at which the valve is spaced. Where conditions favor retaining the mainline or mainlines connecting the jackets or valves, the stumps would generally not be anastomosed but tied off with suture, flow then passing through the bypass permanently as a prosthesis where, for example, the vessels are malacotic or otherwise impaired.

If so, the drugline (sideline, accessory channel feeding line) of the donor jacket is attached to the drug reservoir implanted in the recipient. If anastomosed, leaving a recipient arterial valve jacket with accessory channel in place allows targeting medication to the anastomosis and the graft organ. A retained venous donor jacket allows targeting the venous anastomosis, and the recipient venous jacket the vein. Inelastic when the segments are intussuscepted, or collapsed, tubing which telescopes is unsuitable for use as bloodlines, or mainlines. All components to include jackets and blood-conducting lines, or bloodlines, and all type bridging, or tie-lines, to include aortic neoarches for placement in a pediatric patient are made to expand with growth.

Thus, after transplantation, the servovalves straddle the free ends of the donor and recipient stumps so that flow is through the bypass which crosses over the ends. At this point, with the valves fully extended to divert all flow, flow is through the bypass. In some cases where, for example, the recipient vessel is diseased and not trusted to resist dehiscence of the anastomosis, the bypass is left in place as a prosthesis with the free ends suture-ed closed and sealed with a surgical cement or sealant. Alternatively, the valves can be fully retracted for zero diversion through the bypass, and the free ends anastomosed, one of the valves with drugline retained for follow-up drug targeting by the disorder response system.

Which valve is retained depends upon the direction of flow over the anastomosis as more likely to require treatment. Removal of the other valve is by brief upstream clamping, snipping off the clamp-which will remove the side-entry hole or ostium- and anastomosing the free ends, preferably, with surgical cyanoacrylate cement kept away from the lumen and microsutures, typically 10-0 nylon with a 140 micron needle. Combined with the targeted release of drugs and/or collateral treatment such as electrical, the control system is able to apply such adjustments as are most likely to achieve functional optimization.

In FIG. 22B, when the graft organ-shown here as a lung—has been cut free from the donor and brought to the recipient whose native organ has likewise been removed, the valves fully open, blood flow continues uninterrupted through the graft organ throughout the procedure. The operator leaves stumps of a length adequate to allow the placement of a valve on each arterial and venous stump. The stumps with blood continuing to flow as to bypass them are now anastomosed and the valve diversion chutes retracted so that blood flows through the graft organ.

The recipient arterial stump is above and the donor arterial stump below the arterial anastomosis above, or upstream to the graft organ at the top of the figure, and the donor stump is above or upstream to the venous anastomosis at the bottom of the figure, with the recipient venous anastomosis at the bottom of the figure. FIG. 22B shows these vessels, for a lung, the pulmonary arteries and veins, shown collectively. Here as with the aortic arch, if space does not allow encirclement of the vessel prior to branching, the branches are valved.

To assure clarity, at the top of the figure, the major vessels supporting the graft organ each require two valves, one on the recipient stump above the arterial supply anastomosis, one for the donor arterial stump below the arterial anastomosis, and at the bottom of the figure, one on the donor stump above the venous anastomosis, and one on the venous stump of the recipient below the venous anastomosis. With blood continuing to flow through the anastomoses, the long bloodlines previously needed to span the distance between the donor and recipient to be either discarded. However, valve removal is justified only when their retention causes discomfort. It is therefore preferred that the bloodlines be disconnected and shortened as will allow their reconnection to the valves which can now be used to switch the flow of blood so as to bypass the anastomoses.

Bypass thus is shown in FIGS. 22B and 35F. Shortening and reconnection to the valves allows using the implanted prosthetic disorder response system to periodically switch from flow through to flow around the anastomoses and graft organ, thus relieving the stumps and anastomoses of any shear stress to expedite healing. Equally important, bypass thus makes it possible to release medication into the anastomotic segments without being washed away. The benefit in flow switchability and drug targetability both is so considerable that removal of the valves and bypasses is best reduced in impact by at least preserving drug targetability.

Drugs such as immunosuppressive, anti-inflammatory, or antimicrobial can now be released into the blood bypassing the graft organ, or targeted directly into the anastomotic segments further to expedite healing and ward off rejection. Note in FIGS. 2, 5, 7, 8, and 10 that the valve accessory channels 8 release drugs into the blood flowing past them when the servovalves close off the lumen to diver blood around the anastomoses and that at the same time, the outlets, or exit pores, of the lower accessory channels 31 below the distal tip of the diversion chutes are positioned to release drugs into the blood bypassed lumen to flow down the luminal wall and wet the anastomoses That is, when blood is diverted through the bypasses around the anastomosed segments, accessory channels, or druglines, 8 release drugs into the bypassing bloodstream, while the lower accessory channels 31 release drugs through their exit pores into the bypassed anastomotic segments.

This allows the implanted prosthetic disorder system to coordinate the opening and closing of the servovalves to allow either the diverted blood or anastomotic segments to be medicated while relieved of the throughflow of blood. Medication of the anastomotic segment with the flow of blood bypassing might also be accomplished by placing simple side-entry jackets as described in U.S. Pat. No. 11,759,186, entitled Ductus Side-entry Jackets and Prosthetic Disorder Response Systems to allow accessory channel or drugline delivery just above the anastomoses; however the lower accessory channels, part number 31 accomplish this without the need for three valves at each anastomosis, so that this option should be reserved for servovalves that can be significantly reduced in cost by eliminating the lower accessory channels which cost more than side-entry jackets.

By the same token, replacing the servovalves with simple side-entry jackets would prohibit switching from anastomosis flow-around to luminal flow, much less cooperation between the implanted drug reservoir gravity or pumps outlets to precisely control the amount of each drug released into the anastomotic segments. As stated above in section 2b(1), long-to-short or the reverse switchable lines and switchable confluence conduits transition the distinction between inter- or intra-corporeal. Given that the total number of valves in a compound vascular bypass heart transplant is 18 or 24, valve replacement should be deferred to a follow-up endoscopic procedure with the patient sentient to identify the sites of discomfort. These are then topically or locally anesthetized for removed and replaced by diversion valves.

Usually and preferably where the donor had died away from a hospital so that an ex vivo perfusion machine, which can be situated beside the recipient, was the best means of preservation available, a compound vascular bypass heart transplant will begin as intercorporeal and end as intracorporeal when the procedure culminates in the placement of the graft organ in the recipient using only the shorter of the two switch lines or confluence conduits.

If later due to growth the longer line must be reconnected, that is accomplished endoscopically without any involvement of another party and remains intracorporeal. FIGS. 32A thru 32G depict the venous flow in a long-to-short switchable bloodline as is needed to span the distance between the donor and the recipient. Since arterial flow is identical except in the opposite direction, this has been omitted from some of the drawing figures for visual clarity.

Inline coupling jackets, addressed below, are used to connect both inter- and intra-corporeal implementations of individual and switchable bloodlines and confluence conduits. Ultimately, both short and long lines and confluence conduits can be implanted ab initio. For example, when the second heart in a switch double heart transplant is placed in the lower body, the longer lines used during reciprocal cross-circulation are simply retained to be implanted. Otherwise, switchable bloodlines ordinarily allow the use of long bloodlines to connect a recipient to a donor over a distance, then shorten the line when the donor organ is harvested and implanted.

With respect to FIGS. 34A and 34B showing a mechanical key to operate the bloodline switches shown in FIGS. 32A thru 32G and 33A and 33B, that instead of the mechanical key shown, radio remote control such as Bluetooth or hard wiring could be used to execute the line switching action required using rotary solenoids, and, in FIGS. 37E and 37F that the small vibratory motors used to actuate the serrated razor saw could be actuated in the same way is considered obvious Manually controlled with bloodline switch keys as shown in FIG. 34A, the application of bloodline switches to organ transplants other than of a heart with its larger number of bloodlines and susceptibility to fibrillation and vasospasm when handled is readily negotiable by an operator even unassisted.

The steps depicted in FIGS. 32A thru 32C performed in the space between the organ donor and recipient, and in FIGS. 34D thru 34F in the recipient an open surgical field, an operator with one or two assistants can still administer an orthotopic heart or heterotopic double heart compound bypass transplant, but would be well served were the process facilitated by through the consolidation afforded by using switchable confluence conduits as shown in FIG. 35B and addressed below in section 2b(1)(b)(b15), entitled Switchable Lines and Confluence Conduits: Long when Intercorporeal, to Short when Intracorporeal, Both Affording Connection of One or More Bloodlines to the Same or a Different Number of Bloodlines with Self-leveling Apportionment of Outflow.

Switchable confluence conduits in lieu of multiple separate bloodlines substantially reduce the need for bloodline switch keys such as shown in FIG. 34A. Nevertheless, automating the process so that all of the bloodline switches are actuated synchronously as commanded by the disorder response system is readily attainable with the application of miniature rotary solenoids of which the armature side shaft extensions fit into each bloodline switch key insert, or line stopper switch square or hex axle-turning head slot 177. This can be accomplished with custom small rotary solenoids producible by numerous companies, to include Brandstrom, Chanzon Best Tong, Delta, Geeplus, Johnson Electric, Kendrion Ellis Kuhnke Controls, Koganei, Ledex, the Lee Company, Magnet Schultz, Marotta Controls, Moog, NSF Controls, Schmersal, Shindengen, and Takano.

Used to lengthen or shorten blood and druglines, expandable lines switches allow a period for unlimited growth in an infant, without the need for revision. Because the flats and vaults configuration of the expandable shorter line or confluence conduit is not reversible, a longer line or conduit is needed during implantation to cover the distance separating the donor and the recipient. The need to revert to a longer line during growth is therefore eliminated; the shorter line, no differently than the growth pattern of the patient is unidirectional.

Ordinality, bloodlines, druglines, and confluence conduits once implanted need only increase in dimensions. Accordingly, the line and conduit switching device imparts the ability to switch from long lines and confluence conduits to short ones for implantation. The need to implant lines and confluence conduits already enlarged to adult size is averted. As all of the procedures described herein, the implantation of lines and confluence conduits is accomplished without interruption in the flow of blood.

### Adaptation of the Expandable Structure for Elevated Blood Pressure

However, placement in a patient with hypertension or a history of hypertension since resolved may call for the addition of short gold plated or parylene coated titanium expansion springs above and below the device to retract the distal end back to the normal size at which the springs are completely unextended should hypertension once again arise and be alleviated. Without a protective coating, titanium will not remain intact over the life of a patient. Small projections on the basic device allow the springs to he readily applied. As specified below in section 2b(2)(2f), since springs would restrain expansion of the prosthesis, the applicability thereof is limited to only fully grown patients.

Expansion with Growth

Accordingly, the fivefold or so increase in size notwithstanding, shorter lines and conduits will usually not be useable mid-transplantation. The alternating circumferential flats and vaults configuration contributes postimplantation expandability to lines or conduits which are about a fifth the size of those longer without the need to reenter endoscopically to replace shorter with longer lines or conduits. In practice, since the longer of the two lines can be of any length, that to be implanted, the shorter, can be made to incorporate the circumferential flats and vaults configuration that allow the length of the nominally shorter line or confluence conduit to extend however long the growth of the patient makes it necessary.

Confluence conduits, addressed in section 2b(1)(b)(b14) to follow, entitled Confluence Conduits: Connection of One or More Bloodlines to Flow through a Common Channel to the Same or a Different Number of Bloodlines with Self-leveling Apportionment of Outflow, represent a second concept, that—provided the distance between donor and recipient to be spanned is not too great—a single nonswitchable confluence conduit as shown in FIG. 35A can be used to connect multiple arteries or veins of donor and recipient organs without the need to directly connect each separately to its counterpart.

Instead, the separate bloodlines can be passed through a single larger conduit with separate outlet lines leading to each corresponding vessel in the other party. Such a confluence conduit, such as one shorter in a switchable confluence conduit can be circumferentially formed as specified for expansion, hence, implanted in an infant to eliminate the need for later reentry and revision. Because in an infant with multiple metabolic defects the number of druglines might be larger than could be implanted, this is a critical advantage.

Implantation of a confluence conduit affords the benefit of allowing treatment of the blood input by its contributory vessels alike; however, excessive bulk, size, and the encroachment upon neighboring tissue of the blood switches at either end of the conduit must be verified as countered to cause the infant discomfort. When a kidney implantation is performed endoscopically or robotically, the use of a confluence conduit does not preclude but may necessitate a larger Rutherford Morison entry incision. Usually, the advantages gained will outweigh the disadvantages. The circumferential vaults remain lax and compliant, bloodlines only filling with blood or druglines with therapeutic agents once the neighboring tissue has grown to accommodate the larger diameter.

This has several advantages, to include elimination of the need to operate with numerous bloodlines where one or a few can serve; the larger internal diameter of the joint line facilitates the insertion of diagnostic, therapeutic, or assistive devices such as an inline pump; the normodistributed apportionment of the outflow lines to their respective vessels when those of donor and recipient are both normal, and the spontaneous self-adaptive reapportionment of outlet volume among the receiving vessels when those entering the conduit are greater in number than those sending or the reverse, as arises when a kidney with accessory, or supernumerary, arteries must be replaced or implanted.

Separate diagnostic or therapeutic devices to affect a particular line are applied to the specific inlet or outlet line to affect the supply territory of the one vessel within the organ rather than to affect the entire organ. Combining the switchable bloodline and confluence conduit concepts yields switchable confluence conduits, which make possible both considerable change in the length of the lines connecting the recipient to the donor while offering the advantages of a confluence conduit, to include distinctions in the number of lines entering and departing the conduit.

The denervation in conventional heart transplantation that would increase the heartrate in response to baroreceptor-detected exertion, for example, is compensated for by blood pressure sensors, especially those positioned at critical points, such as along the pulmonary arteries. Transplantation of the heart is more complex than that of other organs, where using the metered switch technique, a vascular servovalve may actually be needed only for the blood supplies to the donor and recipient organs. Additional valves, then, may be used to tap off flow to avoid shear stress, or to gain immediate proximity to the substrate ductus for the release of drugs through the valve accessory channel or channels, for example, where valving is necessary so that a simple drug delivery line would be inadequate.

In a single or double heart transplant, high and low blood pressure detected by implanted pressure sensors can be automatically adjusted to normotensive with the aid of diversion servovalve-connected shunts leading to or from larger extracardiac vessels. The heart is unique in that it receives desaturated blood through the venae cavae, and in that this venous rather than arterial blood ‘supply’ arrives through two great veins rather than a single vessel, or as in the lung, branches thereof. Thus, orthotopic switch transplantation of a replacement heart or heterotopic addition of a second heart benefits from the use of servovalves on both the superior and inferior venae cavae, these valves usually responsive to adjustments commanded by the implanted microprocessor signaled by thin film strip piezoresistive strain gauge pressure sensors at different levels along the internal surface of the synthetic bypass or conduit.

Other than with the heart, such as in a switch heterotopic transplantation of a kidney, a single servovalve on the donor supply bloodline will usually be sufficient to apportion flow between the donor and recipient organs or nearly all flow to either, zero blood supply to be avoided as injurious. When the native organ of the recipient is retained and the donor organ added to create a doublet, the valve can be used to direct all blood to the one or to the other, facilitating the removal of either. In switch transplantation of other organs and glands, such as the kidneys, where multiple major in- and central outflow orifices are not involved in ejection and venous return, the valving required is much simpler.

One servovalve on the donor renal artery is needed. Connection of the venous bloodline—the line connecting donor and recipient renal veins—is ordinarily with basic ductus side-entry jackets incorporating a diversion chute inside the jacket side tubes molded in a suitable material for making jacket and valve parts such as polyether ether ketone. Unless routing the lines to a valve jacket is problematic, druglines to the jacket accessory channel or channels are best run alongside the corresponding bloodlines, or mainlines.

When the distance between the valves is larger so that the weight of both would cause no discomfort, and the pair would achieve more extended coverage of the tissue to be monitored and treated as necessary postprocedurally, then both are left in place. If not, then one of the valves with its mainline and one or more druglines is removed and the stumps anastomosed. A partial ureter cannot be successfully continued by inserting a catheter inside its distal end—where the encircling diversion jacket is strongly resistant to migration and provided with one or more accessory channels, peristalsis will quickly dislodge a catheter. Once the graft organ is orthotopically positioned, if the bypass is to remain as a prosthesis, the free ends of the donor and recipient stumps are sealed; if not these are anastomosed.

When proximity between donor and recipient jackets or valves results in needless bulk and/or encroaches upon neighboring tissue as to either demand adjustment to allow use of the bypass as a prosthesis or the removal of either or both jackets disallowing such use. Jacket removal and resituation along the substrate ductus is accomplished expeditiously not by detachment and reattachment but by clamping, excision, anastomosis, then stitching the jacket with attached lines into the new level, the same remedy used to rotate a valve jacket. Most adjustments and revisions can be accomplished endoscopically or through a combination of endoscopic and transluminal techniques.

Accordingly, the need to remove a jacket or valve by separating it from the substrate ductus should seldom if ever be needed. If some exceptional circumstance were to override this, removal with minimal loss of blood is more expeditiously accomplished by brief clamping, or in an open field, manual compression, and if the jacket or valve jacket is just short of a transection, the jacket or valve jacket and the mainline entry opening, or surgical ostium created by the trepan, or trephine, simply snipped off and the free end sealed or anastomosed.

A less invasive option is the use a vascular closure device (see, for example, Saleem, T. and Baril, D. T. 2020. “Vascular Access Closure Devices,” Online, Treasure Island, Florida: StatPearls Publishing, at https://www.ncbi.nlm.nih.gov/books/NBK470233/; Kim, S. H., Behnes, M., Baron, S., Shchetynska-Marinova. T., Tekinsoy, M., and 4 others 2019. “Differences of Bleedings after Percutaneous Coronary Intervention Using Femoral Closure and Radial Compression Devices,” Medicine (Baltimore, Maryland) 98(20): e15501; Kim, S. H., Behnes, M., Baron, S., Shchetynska-Marinova. T., Uensal. M., and 4 others 2019. “Extravascular Compared to Intravascular Femoral Closure is Associated with Less Bleeding and Similar MACE [major adverse cardiac events] after Percutaneous Coronary Intervention,” International Journal of Medical Sciences (Sydney, Australia) 16(1): 43-50; Smith, J. C., Monroe, E. J., Shivaram, G. M., Shaw, D. W. W., and Koo, K. S. H. 2019. “An Update on the Use of an Arterial Closure Device following Femoral Arterial Puncture in Children,” Pediatric Radiology 49(9): 1217-1221; Giordano, A., Messina, S., Maresca, G., and Biondi-Zoccai, G. 2018. “Hemostasis after Brachial Artery Access with the MynxGrip Device: A Case Report,” Clinical Medicine Insights. Cardiology 12:1179546818759298; Hoffmann, P., Al-Ani, A., von Lueder, T., Skoe, O. W., Tran, T. T., and Opdahl, A. 2018. “Late Pseudoaneurysm after Access Site Closure with Manta in Transfemoral Aortic Valve Implantation,” European Journal of Vascular and Endovascular Surgery Short Reports 42:34-36; Noori, V. J. and Eldrup-Jorgensen, J. 2018. “A Systematic Review of Vascular Closure Devices for Femoral Artery Puncture Sites,” Journal of Vascular Surgery (New York, New York) 68(3): 887-899; Scott, M. C., Spencer, H. J., Ali, A. T., Moursi, M. M., Escobar, G. A., Lyons, L. C., and Smeds, M. R. 2018. “Mynx Vascular Closure Device in Arterial Endovascular Procedures,” Annals of Vascular Surgery 46:112-117; Sartorius, B., Behnes, M., Unsal, M., Hoffmann, U., Lang. S., and 3 others 2017. “Arterial Access-site Complications after Use of a Vascular Closure Device Related to Puncture Height,” BioMed Central Cardiovascular Disorders (London, England) 17(1): 64; Van Mieghem, N. M., Latib. A., van der Heyden, J., van Gils, L., Daemen, J., and 10 others 2017. “Percutaneous Plug-based Arteriotomy Closure Device for Large-bore Access: A Multicenter Prospective Study,” Journal of the American College of Cardiology. Cardiovascular Interventions 10(6): 613-619; Rao. S. V. and Stone, G. W. 2016. “Arterial Access and Arteriotomy Site Closure Devices,” Nature Reviews. Cardiology 13(11): 641-650; Cox, T., Blair, L., Huntington, C., Lincourt. A., Sing. R., and Heniford, B. T. 2015. “Systematic Review of Randomized Controlled Trials Comparing Manual Compression to Vascular Closure Devices for Diagnostic and Therapeutic Arterial Procedures,” Surgical Technology International 27:32-44).

The rerouting of lines is best by brief detachment, that of mainlines conducting blood requiring brief clamping. The practical absolute distance for such a move negligible, for this to necessitate replacement of the highly stretchable lines as too short should not arise. The removal of a ductus side-entry or valve jacket and mainline, which are essential to the bypass and to allowing it to remain in place as a prosthesis, requires that the free ends of the stumps be anastomosed.

If the catheter could be stabilized, it would eventually fail anyway, if not due to an adverse tissue reaction for which an unsupported catheter offers no defense, then due to encrustation and/or the formation of a biofilm. That fresh urine is sterile is mistaken—the bladder of a healthy person harbors uropathogenic microbiota (see, for example, Ackerman, A. L., Anger, J. T., Khalique, M. U., Ackerman, J. E., Tang. J., and 3 others 2019. “Optimization of DNA Extraction from Human Urinary Samples for Mycobiome Community Profiling,” Online, PLoS [Public Library of Science] One 14(4): e0210306; Buhmann, M. T., Abt. D., Nolte, O., Neu, T. R., Strempel, S., and 6 others 2019. “Encrustations on Ureteral Stents from Patients without Urinary Tract Infection Reveal Distinct Urotypes and a Low Bacterial Load” Microbiome (London, England) 7(1): 60; Thanert, R., Reske, K. A., Hink, T., Wallace, M. A., Wang, B., and 7 others 2019. “Comparative Genomics of Antibiotic-resistant Uropathogens Implicates Three Routes for Recurrence of Urinary Tract Infections,” MBio [Microbiology] 10(4). pii: e01977-19; Mueller, E. R., Wolfe, A. J., and Brubaker, L. 2017. “Female Urinary Microbiota,” Current Opinion in Urology 27(3): 282-286; Tang, J. 2017. “Microbiome in the Urinary System-A Review,” American Institute of Mathematical Sciences Microbiology 3(2): 143-154; Wolfe, A. J. and Brubaker, L. 2015. “Sterile Urine” and the Presence of Bacteria,” European Urology 68(2): 173-174).

By contrast, the accessory channel associated with a side-entry jacket affords a direct path to eradicate any cause of obstruction whether the agent called for is an anticoagulant, antibiotic, antimycotic, and/or an anti-inflammatory. The implanted automatic disorder response system that ductus side-entry jackets, nonjacketing side-entry connectors, and the various valves and servovalves described make possible can directly pipeline-target antimicrobials, for example, to the ureters or bladder, for example.

The drug is stored in a small flat reservoir placed subdermally in the pectoral region, and an implant microcontroller executing its prescription-program automatically triggers the reservoir outlet pump or drip stopcock remote control device to release the drug. The advantages of immediate access to a root, nidus, or site, of disease of disease for diagnostic and therapeutic purposes cannot be overstated. Also important is that in a regimen adherent-undependable patient, an implanted disorder response system can automatically deliver medication directly to any such site.

That the drug, an antibiotic or antibiotics, for example, is tightly targeted- and if necessary, can be eliminated from continuing through the circulation in several ways, some specified below in this section-reduces if not eliminates the development of resistance to it, safeguards the gut microbiota, preventing disruption to the microbiome-gut-brain axis and the numerous neuropsychological, hepatic, and other consequences to which this can give rise (see, for example, Akhondzadeh, S. 2019. “Microbiota and Autism Spectrum Disorder,” Avicenna Journal of Medical Biotechnology (Tehran, Iran) 11(2): 129; Amedei, A. 2019. “Potential Therapeutic Strategies to Target Gut Microbiota in Hepatocellular Carcinoma,” Hepatobiliary Surgery and Nutrition (Hong Kong, China) 8(5): 527-529; Angelucci, F., Cechova, K., Amlerova, J., and Hort, J. 2019. “Antibiotics, Gut Microbiota, and Alzheimer's Disease,” Journal of Neuroinflammation 16(1): 108; Armour, C. R., Nayfach, S., Pollard, K. S., and Sharpton, T. J. 2019. “A Metagenomic Meta-analysis Reveals Functional Signatures of Health and Disease in the Human Gut Microbiome,” mSystems 4(4). pii: e00332-18; Chen, X., Li, H. Y., Hu, X. M., Zhang, Y., and Zhang, S. Y. 2019. “Current Understanding of Gut Microbiota Alterations and Related Therapeutic Intervention Strategies in Heart Failure,” Chinese Medical Journal (English edition) 132(15): 1843-1855; Derovs. A., Laivacuma. S., and Krumina, A. 2019. “Targeting Microbiota: What Do We Know about It at Present?,” Medicina (Kaunas, Lithuania) 55(8). pii: E459; Fattorusso, A., Di Genova, L., Dell'Isola, G. B., Mencaroni, E., and Esposito, S. 2019. “Autism Spectrum Disorders and the Gut Microbiota,” Nutrients 11(3). pii: E521; Gorecki, A. M., Preskey, L., Bakeberg, M. C., Kenna, J. E., Gildenhuys, C., and 6 others 2019. “Altered Gut Microbiome in Parkinson's Disease and the Influence of Lipopolysaccharide in a Human α-Synuclein Over-expressing Mouse Model,” Online, Frontiers in Neuroscience 13:839; Harikrishnan, S. 2019. “Diet, the Gut Microbiome and Heart Failure,” Cardiac Failure Review 5(2): 119-122; Hirschberg. S., Gisevius. B., Duscha. A., and Haghikia, A. 2019. “Implications of Diet and The Gut Microbiome in Neuroinflammatory and Neurodegenerative Diseases,” Online, MDPI [Multidisciplinary Digital Publishing Institute] International Journal of Molecular Sciences (Basel, Switzerland) 20(12). pii: E3109; Hu X., Xing. M., An, S., Wan, Y., and Liu, Q. 2019. “The Gut Microbiota and Heart Disease Prevention,” European Journal of Preventive Cardiology 26(1): 109; Kowalski, K. and Mulak, A. 2019. “Brain-Gut- Microbiota Axis in Alzheimer's Disease,” Journal of Neurogastroenterology and Motility 25(1): 48-60; Milosevic, I., Vujovic, A., Barac. A., Djelic. M., Korac. M., and 7 others 2019. “Gut- Liver Axis, Gut Microbiota, and Its Modulation in the Management of Liver Diseases: A Review of the Literature,” Online, MDPI [Multidisciplinary Digital Publishing Institute] International Journal of Molecular Sciences (Basel, Switzerland) 20(2). pii: E395; Miraglia, F. and Colla, E. 2019. “Microbiome, Parkinson's Disease and Molecular Mimicry,” Cells 8(3). pii: E222; Yu, J., Fan, Y., Wang. L., Huang. Y., Xia, J., and 8 others 2019. “Intestinal Surgery Contributes to Acute Cerebellar Ataxia through Gut Brain Axis,” Online, Frontiers in Neurology 10:995; Bu. J. and Wang, Z. 2018. “Cross-talk between Gut Microbiota and Heart via the Routes of Metabolite and Immunity,” Gastroenterology Research and Practice 2018:6458094; Dopkins, N., Nagarkatti, P. S., and Nagarkatti, M. 2018. “The Role of Gut Microbiome and Associated Metabolome in the Regulation of Neuroinflammation in Multiple Sclerosis and its Implications in Attenuating Chronic Inflammation in Other Inflammatory and Autoimmune Disorders,” Immunology 154(2): 178-185; Logsdon, A. F., Erickson, M. A., Rhea, E. M., Salameh. T. S., and Banks, W. A. 2018. “Gut Reactions: How the Blood-Brain Barrier Connects the Microbiome and the Brain,” Experimental Biology and Medicine (Maywood, New Jersey) 243(2): 159-165; Spielman, L. J., Gibson, D. L., and Klegeris. A. 2018. “Unhealthy Gut, Unhealthy Brain: The Role of the Intestinal Microbiota in Neurodegenerative Diseases,” Neurochemistry International 120:149-163; Yang, Q., Lin, S. L., Kwok, M. K., Leung. G. M., and Schooling, C. M. 2018. “The Roles of 27 Genera of Human Gut Microbiota in Ischemic Heart Disease, Type 2 Diabetes Mellitus, and Their Risk Factors: A Mendelian Randomization Study,” American Journal of Epidemiology 187(9): 1916-1922; Yoshida, N., Yamashita, T., and Hirata, K. I. 2018. “Gut Microbiome and Cardiovascular Diseases,” Diseases (Basel, Switzerland) 6(3). pii: E56; Jiang, C., Li, G., Huang, P., Liu, Z., and Zhao, B. 2017. “The Gut Microbiota and Alzheimer's Disease,” Journal of Alzheimer's Disease 58(1): 1-15; Schneiderhan, J., Master-Hunter. T., and Locke, A. 2016. “Targeting Gut Flora to Treat and Prevent Disease,” Journal of Family Practice 65(1): 34-38; Online at www.mdedge.com/familymedicine/article/105514/gastroenterology/targeting-gut-flora-treat- and-prevent-disease; Bajaj. J. S., Betrapally, N. S., Hylemon, P. B., Heuman, D. M., Daita, K., and 7 others 2015. “Salivary Microbiota Reflects Changes in Gut Microbiota in Cirrhosis with Hepatic Encephalopathy,” Hepatology 62(4): 1260-1271; Rai. R., Saraswat, V. A., and Dhiman, R. K. 2015. “Gut Microbiota: Its Role in Hepatic Encephalopathy,” Journal of Clinical and Experimental Hepatology 5 (Supplement 1): S29-536; Bajaj. J. S., Hylemon, P. B., Ridlon, J. M., Heuman, D. M., Daita, K., and 5 others 2012. “Colonic Mucosal Microbiome Differs from Stool Microbiome in Cirrhosis and Hepatic Encephalopathy and is Linked to Cognition and Inflammation,” American Journal of Physiology. Gastrointestinal and Liver Physiology 303(6): G675-G685).

Moreover, that administration is automatic protects regimen noncompliant patients, whether very young, of diminished cognitive capacity, psychopathological, or physically disabled (see, for example, Fitzpatrick, M. A., Suda, K. J., Safdar, N., Burns, S. P., Jones, M. M., and 3 others 2018. “Changes in Bacterial Epidemiology and Antibiotic Resistance among Veterans with Spinal Cord Injury/Disorder over the Past 9 Years,” Journal of Spinal Cord Medicine 41(2): 199-207; Tofte, N., Nielsen, A. C., Trostrup. H., Andersen, C. B., Von Linstow, M., and 4 others 2017. “Chronic Urinary Tract Infections in Patients with Spinal Cord Lesions-Biofilm Infection with Need for Long-term Antibiotic Treatment,” Acta Pathologica, Microbiologica, et Immunologica Scandinavica 125(4): 385-391; Ploypetch, T., Dajpratham, P., Assanasen, S., Thanakiatpinyo. T., Tanvijit, P., and Karawek, J. 2013. “Epidemiology of Urinary Tract Infection among Spinal Cord Injured Patients in Rehabilitation Ward at Siriraj Hospital,” Journal of the Medical Association of Thailand 96(1): 99-106; Tantisiriwat, N., Kittisomprayoonkul, W., Sukonthamarn, K., Unhasuta, C., Suankratay, C., Tantisiriwat, W., and Aksaranugraha, S. 2007. “Uropathogens and Empiric Antibiotics for the Treatment of Urinary Tract Infections in Spinal Cord Injured Patients at Rehabilitation Center, Thai Red Cross Society during 2001 to 2005,” Journal of the Medical Association of Thailand 90(11): 2482-2486).

For example, methicillin resistant Staphylococcus aureus and Staphylococcus epidermidis and the biofilms these produce have been found to yield to thiazolidine derivatives at nonhemolytic concentrations (see, for example, Velikova, N., Fulle, S., Manso, A. S., Mechkarska, M., Finn, P., and 4 others 2016. “Putative Histidine Kinase Inhibitors with Antibacterial Effect Against Multi-drug Resistant Clinical Isolates Identified by in Vitro and in Silico Screens,” (Nature Research) Scientific Reports 6:26085; Liu, H., Zhao, Y., Zhao, D., Gong. T., Wu. Y., Han, H., and 4 others 2015. “Antibacterial and Anti-biofilm Activities of Thiazolidione Derivatives Against Clinical Staphylococcus Strains,” Emerging Microbes and Infections 4(1): e1; Bem, A. E., Velikova, N., Pellicer, M. T., Baarlen, P., Marina, A., and Wells, J. M. 2015. “Bacterial Histidine Kinases as Novel Antibacterial Drug Targets,” ACS [American Chemical Society] Chemical Biology 10(1): 213-224; Liu, H., Zhao, D., Chang, J., Yan, L., Zhao, F., Wu. Y., Xu. T., and 6 others 2014. “Efficacy of Novel Antibacterial [thiazolidine core structure derivative] Compounds Targeting Histidine Kinase YycG Protein,” Applied Microbiology and Biotechnology 2014 98(13): 6003-6013; Zhao, D., Chen, C., Liu, H., Zheng. L., Tong. Y., Qu. D., and Han, S. 2014. “Biological Evaluation of Halogenated Thiazolo[3,2-a]pyrimidin-3-one Carboxylic Acid Derivatives Targeting the YycG Histidine Kinase,” European Journal of Medicinal Chemistry 87:500-507; Huang, R. Z., Zheng, L. K., Liu, H. Y., Pan, B., Hu. J., and 7 others 2012. “Thiazolidine Derivatives Targeting the Histidine Kinase YycG are Effective Against Both Planktonic and Biofilm-associated Staphylococcus epidermidis,” Acta Pharmacologica Sinica 33(3): 418-425; Qin, Z., Zhang, J., Xu. B., Chen, L., Wu. Y., and 6 others 2006. “Structure-based Discovery of Inhibitors of the YycG Histidine Kinase: New Chemical Leads to Combat Staphylococcus epidermidis Infections,” Online, BioMed Central Microbiology 6:96).

Microbial intrusion is dispelled not only through the use of antimicrobial material, but if necessary, the directly targeted delivery of an antimicrobial through the valves accessory channels. The synthetics used in the catheters can be prepared to resist intrusion and colonization by many biofilm-producing bacteria. Materials that release the antibacterial agent or agents only when actually in contact with a pathogen, or infectious agent, the release thereof is not likely to continue over an indefinite period as required for the permanent parts of the valves. For implantation on a long-term basis, thromboresistance based upon the intrinsic properties of the materials used is preferable; however, for long-term use, it is possible to include a small replenishable or replaceable canister in a surface port to release nitric oxide when commanded by the control microprocessor.

Such could, however, prove adequate for catheters and end-tools to be inserted into or through the valves for diagnostic purposes or for temporary use (see, for example, Rong. F., Tang. Y., Wang, T., Feng. T., Song, J., Li, P., and Huang. W. 2019. “Nitric Oxide-releasing Polymeric Materials for Antimicrobial Applications: A Review,” Antioxidants (Basel, Switzerland) 8(11). pii: E556; Yañez-Macias, R., Muñoz-Bonilla, A., De Jesus-Tellez, M. A., Maldonado-Textle, H., Guerrero-Sanchez, C., Schubert, U.S., and Guerrero-Santos, R. 2019. “Combinations of Antimicrobial Polymers with Nanomaterials and Bioactives to Improve Biocidal Therapies,” Polymers (Basel, Switzerland) 11(11). pii: E1789; Zhou, Y., Tan, J., Wu, J., Zhang, Q., Andre, J., and 3 others 2019. “Nitric Oxide Releasing Poly(vinylidene Fluoride-co-hexafluoropropylene) Films Using a Fluorinated Nitric Oxide Donor to Greatly Decrease Chemical Leaching,” Acta Biomaterialia 90:112-121: Luo. C., Liu, W., Luo, B., Tian, J., Wen, W., Liu. M., and Zhou, C. 2017. “Antibacterial Activity and Cytocompatibility of Chitooligosaccharide-modified Polyurethane Membrane via Polydopamine Adhesive Layer,” Carbohydrate Polymers 156:235-243; Pant. J., Gao, J., Goudie, M. J., Hopkins, S. P., Locklin, J., and Handa, H. 2017. “A Multi-defense Strategy: Enhancing Bactericidal Activity of a Medical Grade Polymer with a Nitric Oxide Donor and Surface-immobilized Quaternary Ammonium Compound,” Acta Biomaterialia 58:421-431; Singha, P., Locklin, J., and Handa, H. 2017. “A Review of the Recent Advances in Antimicrobial Coatings for Urinary Catheters,” Acta Biomaterialia 50:20-40; Xu. D., Su, Y., Zhao, L., Meng. F., Liu, C., Guan, Y., Zhang. J., and Luo J. 2017. “Antibacterial and Antifouling Properties of a Polyurethane Surface Modified with Perfluoroalkyl and Silver Nanoparticles,” Journal of Biomedical Materials Research. Part A 105(2): 531-538; Cai. T., Gallelli, L., Meacci, F., Brugnolli, A., Prosperi, L., and 7 others 2016. “The Efficacy of Umbelliferone, Arbutin, and N-Acetylcysteine to Prevent Microbial Colonization and Biofilm Development on Urinary Catheter Surface: Results from a Preliminary Study,” Journal of Pathogens 2016: 1590952′; Coad, B. R., Griesser, H. J., Peleg. A. Y., and Traven, A. 2016. “Anti-infective Surface Coatings: Design and Therapeutic Promise against Device-Associated Infections,” Online, PLoS [Public Library of Science] Pathogens 12(6): e1005598; Damodaran, V. B. and Murthy, N. S. 2016. “Bio-inspired Strategies for Designing Antifouling Biomaterials,” Biomaterials Research 20:18; Liu, C. Y. and Huang, C. J. 2016. “Functionalization of Polydopamine via the Aza-Michael Reaction for Antimicrobial Interfaces,” Langmuir: The ACS [American Chemical Society] Journal of Surfaces and Colloids 32(19): 5019-5028; Huang, K. S., Yang, C. H., Huang, S. L., Chen, C. Y., Lu, Y. Y., and Lin, Y. S. 2016. “Recent Advances in Antimicrobial Polymers: A Mini-Review,” Online, MDPI [Multidisciplinary Digital Publishing Institute] International Journal of Molecular Sciences (Basel, Switzerland) 17(9), pii: E1578; Junter, G. A., Thebaul. P., and Lebrun, L. 2016. “Polysaccharide-based Antibiofilm Surfaces,” Acta Biomaterialia 30:13-25; Kwok, K., Chang, J., Lo, S., Giap. A., Lim, B., and Wu, B. 2016. A Novel Adjunctive Cleansing Method to Reduce Colony-forming Units on Duodenoscopes,” Endoscopy International Open 4(11): E1178-E1182; Macdonald, T. J., Wu, K., Sehmi, S. K., Noimark, S., Peveler, W. J., and 6 others 2016. “Thiol-capped Gold Nanoparticles Swell-encapsulated into Polyurethane as Powerful Antibacterial Surfaces Under Dark and Light Conditions,” (Nature Research) Scientific Reports 6:39272; Rai, A., Pinto, S., Evangelista, M. B., Gil, H., Kallip. S., Ferreira, M. G., and Ferreira, L. 2016. “High-density Antimicrobial Peptide Coating with Broad Activity and Low Cytotoxicity Against Human Cells,” Acta Biomaterialia 33:64-77; Sehmi, S. K., Allan, E., MacRobert, A. J., and Parkin, I. 2016. “The Bactericidal Activity of Glutaraldehyde-impregnated Polyurethane,” AficrobiologyOpen 5(5): 891-897; Velikova N., Mas, N., Miguel-Romero, L., Polo, L., Stolte, E., and 7 others 2016. “Broadening the Antibacterial Spectrum of Histidine Kinase Autophosphorylation Inhibitors via the Use of ε-poly-L-lysine Capped Mesoporous Silica-based Nanoparticles,” Nanomedicine 13(2): 569-581: Wo. Y., Brisbois, E., Bartlett, R. H., and Meyerhoff, M. E. 2016. “Recent Advances in Thromboresistant and Antimicrobial Polymers for Biomedical Applications: Just Say Yes to Nitric Oxide (NO),” Biomaterials Science 4(8): 1161-1183; Boda, S. K., Broda, J., Schiefer, F., Weber-Heynemann, J., and 4 others 2015. “Cytotoxicity of Ultrasmall Gold Nanoparticles on Planktonic and Biofilm Encapsulated Gram-positive Staphylococci,” Small (Weinheim an der Bergstrasse, Germany) 11(26): 3183-3193; Lim, K., Chua, R. R., Bow, H., Tambyah, P. A., Hadinoto, K., and Leong, S. S. 2015. “Development of a Catheter Functionalized by a Polydopamine Peptide Coating with Antimicrobial and Antibiofilm Properties,” Acta Biomaterialia 15:127-138; Sehmi, S. K., Noimark, S., Weiner, J., Allan, E., MacRobert, A. J., and Parkin, I. P. 2015. “Potent Antibacterial Activity of Copper Embedded into Silicone and Polyurethane,” ACS [American Chemical Society] Applied Materials and Interfaces 7(41): 22807-22813; Kara, F., Aksoy, E. A., Yuksekdag, Z., Hasirci, N., and Aksoy, S. 2014. “Synthesis and Surface Modification of Polyurethanes with Chitosan for Antibacterial Properties,” Carbohydrate Polymers 112:39-47; Lam, T. B., Omar, M. I., Fisher, E., Gillies, K., and MacLennan, S. 2014. “Types of Indwelling Urethral Catheters for Short-term Catheterisation in Hospitalised Adults,” Cochrane Database of Systematic Reviews(9): CD004013; Pelgrift, R. Y. and Friedman, A. J. 2013. “Nanotechnology as a Therapeutic Tool to Combat Microbial Resistance,” Advanced Drug Delivery Reviews 65(13-14): 1803-1815; Quah, S. Y., Wu. S., Lui, J. N., Sum, C. P., and Tan, K. S. 2012. “N-acetylcysteine Inhibits Growth and Eradicates Biofilm of Enterococcus faecalis,” Journal of Endodontics38(1): 81-85; Wang, Y., Hong, Q., Chen. Y., Lian, X., and Xiong. Y. 2012. “Surface Properties of Polyurethanes Modified by Bioactive Polysaccharide-based Polyelectrolyte Multilayers,” Colloids and Surfaces, B. Biointerfaces 100:77-83).

Recent advances at incorporating antimicrobial agents into the material of catheters addressed here complement the ability to pipeline target antimicrobials to the treatment site through the accessory channels, coating the internal surfaces of the channels and wetting the tissue at the terminus, enhancing the ability to implant catheters for long-term use. References pertaining to some of these recent developments are cited above. Tissue engineered vascular tissue will eventually supplant the materials currently used for prosthetic ductus. The lines are made of debris accumulation-resistant materials, and whether synthetic or tissue engineered, the use of ductus side-entry jackets to join such lines to native ductus allows the lines, valves, and graft to be directly pipeline-targeted for maintenance agents to prevent clotting, crystallization, and biofilm.

The main complication in the use of small valves to steer the bloodstream—the risk of occlusion due to a buildup of clot—is dispelled through the delivery of a fractionated heparin drip through the valve accessory channels and by making the lines and valves of clot-resistant materials. Rare instances to the contrary (Blas, J. V., Carsten, C. G. 3rd, and Gray, B. H. 2016. “Heparin-induced Thrombocytopenia Associated with a Heparin-bonded Stent-graft,” Annals of Vascular Surgery 33:227.e1-e4; Kasirajan, K. 2012. “Outcomes after Heparin-induced Thrombocytopenia in Patients with Propaten [Gore® PROPATEN,® W. L. Gore & Associates, Newark, Delaware] Vascular Grafts,” Annals of Vascular Surgery 26(6): 802-808; Gabrielli, R., Siani, A., Rosati, M. S., Antonelli, R., Accrocca, F., Giordano, G. A., and Marcucci, G. 2011. “Heparin-induced Thrombocytopenia Type II Because of Heparin-coated Polytetrafluoroethylene Graft Used to Bypass,” Annals of Vascular Surgery 25(6): 840.e9-e12), in limited and localized doses, the risk of inducing thrombocytopenia is improbable.

Moreover, heparin-coated lines are also positively endorsed (see, for example, Lumsden, A. B. and Morrissey, N. J. with 25 collaborators 2015. “Randomized Controlled Trial Comparing the Safety and Efficacy between the FUSION BIOLINE [Getinge Group, Gothenburg, Sweden] Heparin-coated Vascular Graft and the Standard Expanded Polytetrafluoroethylene Graft for Femoropopliteal Bypass,” Journal of Vascular Surgery (New York, New York) 61(3): 703-712.e1). An alternative to the use of synthetic vessels incorporating heparin is the release of another anticoagulant of which there are many, such as warfarin. Seen thus, heparin bonding is merely a convenience. Antimicrobial polymers for biomedical applications are addressed just above (see, for example, Wo, Y., Brisbois, E., Bartlett, R. H., and Meyerhoff, M. E. 2016, Op cit.). Along the urinary tract, the risk of occlusion due to a buildup of crystal is dispelled through the delivery of a crystal solvent, addressed in section 2c, entitled Ductus Side-entry Jackets and Vascular Valves in Urinary Diversion

Materials that release thromboresistant substances have a limited life, while those that consist of or are coated by highly slippery materials, especially when supplemented by the direct pipeline-targeting of a heparin, for example, can remain in place for years if not life (see, for example, Hyun D Y, Han X, Park D S, Kim M, Park J K, and 5 others 2023. “A Novel Polymer-free Everolimus-eluting Stent with a Nitrogen-doped Titanium Dioxide Film Inhibits Restenosis and Thrombosis in a Swine Coronary Model,” Online, Cardiology Journal (Gdańsk, Poland) 30(6): 881-891; Torii, S., Cheng, Q., Mori, H., Lipinski, M. J., Acampado, E., and 6 others 2019. “Acute Thrombogenicity of Fluoropolymer-coated versus Biodegradable and Polymer-free Stents,” EuroIntervention (Toulouse, France) 14(16): 1685-1693; Liisberg, M., Stenger, M., Behr-Rasmussen, C., Stubbe, J., and Lindholt, J. S. 2018. “Experimental Comparative Study of Thrombogenicity of Two Differently Luminal Heparinized ePTFE [expanded polytetrafluoroethylene] Vascular Prosthetics,” Annals of Medicine and Surgery(2012) (London, England) 35:76-81; Schaarschmidt, B. M., Boos, J., Buchbender, C., Kropil, P., Kropil, F., and 5 others 2018. “Heparin-bonded Stent-graft Treatment for Major Visceral Arterial Injury after Upper Abdominal Surgery,” European Radiology 28(8): 3221-3227; Heyligers, J. M., Lisman, T., Verhagen, H. J., Weeterings, C., de Groot, P. G., and Moll, F. L. 2008. “A Heparin-bonded Vascular Graft Generates No Systemic Effect on Markers of Hemostasis Activation or Detectable Heparin-induced Thrombocytopenia-associated Antibodies in Humans,” Journal of Vascular Surgery (New York, New York) 47(2): 324-329; Jordan, S. W. and Chaikof, E. L. 2007. “Novel Thromboresistant Materials,” Journal of Vascular Surgery (New York, New York) 45 Supplement A: A104-A115).

This does, however, presuppose that targeted rather than systemic agents are preferable and that the placement of a subdermal port just beneath the dermis is justified. Untenably susceptible to occlusion without the ability to directly target antithrombotic, antimicrobial, and crystal dissolving agents to the sites of fouling, synthetics are not used to replace small caliber ductus. For this reason, the conventional use of synthetics is limited to large caliber ductus, specifically, in aortofemoral, aortobifemoral, aortoiliac, iliofemoral, and femoropopliteal bypass grafts.

The incentive to use synthetic tubing harbored the implicit desire for full implantation whereby no piping of medication from a port at the body surface to the graft would be needed. However, it is now possible to establish an automatically administered internal drip of essential agents where periodic replenishment is by jet injection through a subdermally or subcutaneously implanted port or subdermally implanted accessory channel hole in the surface port.

When the same type diversion jacket is applied to the diversion of blood from an artery too small for end-to-end anastomosis with a length of synthetic tubing to terminate just beneath the affected surface, such as an ulcer of the crus, or shank, or foot, the accessory channel with a small pump or drip stopcock remote control device under the control of a timing module or microcontroller is used to drip an anticoagulant into the line, typically low molecular weight or unfractionated heparin or vitamin K antagonists with careful monitoring of any patient with impaired kidney function, who would then be given a lower dose of low molecular weight heparin.

As described in U.S. Pat. No. 11,013,858, entitled Nonjacketing Side-entry Connectors and Prosthetic Disorder Response Systems, and U.S. Pat. No. 11,759,186, entitled Ductus Side-entry Jackets and Prosthetic Disorder Response Systems, the distal terminus at the hypoxic site is configured to effect reperfusion after Vineberg's method for treatment of the under-oxygenated heart. Such a service channel can also be positioned proximal to a smaller caliber synthetic catheter placed along a native blood vessel or in the urinary tract, or a synthetic (silicone) neobladder to prevent rapid incrustation (encrustation) (Stein, R., Hohenfellner, M., Pahemik, S., Roth, S., Thuroff, J. W., and Rubben, H. 2012. “Urinary Diversion-Approaches and Consequences,” Deutsches Ärzteblatt [German Medical Journal] International 109(38): 617-622) to directly target the counteractant to the synthetic component.

Combining current split thickness skin grafting procedure to close over the ulcerated surface (see, for example, Kneilling, M., Breuninger, H., Schippert, W., Hafner, H. M., and Moehrle, M. 2011. “A Modified, Improved, Easy and Fast Technique for Split-thickness Skin Grafting,” British Journal of Dermatology 165(3): 581-584, reproduced in Medscape Friday, Jan. 6, 2017 at http://www.medscape.com/viewarticle/748961_4; McCartan, B. and Dinh, T. 2012. “The Use of Split-thickness Skin Grafts on Diabetic Foot Ulcerations: A Literature Review,” Plastic Surgery International 2012:715273; Kirsner, R. S., Eaglstein. W. H., and Kerdel, F. A. 1997. “Split-thickness Skin Grafting for Lower Extremity Ulcerations,” Dermatologic Surgery 23(2): 85-93) with direct perfusion by the diversion of oxygenated blood as indicated, facilitates the healing of a venous stasis ulcer, for example, and does so with results esthetically superior to older pinch graft methods (see, for example, Wheeland, R. G. 1987. “The Technique and Current Status of Pinch Grafting,” Journal of Dermatologic Surgery and Oncology 13(8): 873-880). Skin grafting aside, the diversion of blood using a synthetic line with heparin support as specified is no less applicable to under-oxygenated tissue, regardless of region.

As will be made clear, when a prospective organ donor dies, is sustained past death in virtually living condition, and a graft organ taken from that donor is transferred into a matched recipient, the infusion reactions, or ‘cytokine storms’ (references cited below in this section), and systemic inflammatory response syndrome associated with organ failure and death will have been considerably prevented from degrading the prospective graft. Broadly, eliminating this deterioration reduces the multiple obstacles of transplantation to one of immune tolerance, and the metered switch, or compound vascular bypass, method to be described reduces this remaining problem as well.

“We have recently demonstrated that extracellular mitochondria are abundant in the circulation of deceased organ donors and that their presence correlates with early allograft dysfunction” (Lin, L., Xu, H., Bishawi, M., Feng, F., Samy, K., and 4 others 2019. “Circulating Mitochondria in Organ Donors Promote Allograft Rejection,” American Journal of Transplantation 19(7): 1917-1929); (see also, for example, Pollara, J., Edwards, R. W., Lin, L., Bendersky, V. A., and Brennan, T. V. 2018. “Circulating Mitochondria in Deceased Organ Donors are Associated with Immune Activation and Early Allograft Dysfunction,” Journal of Clinical Investigation Insight 3(15). pi: 121622; Scozzi, D., Ibrahim, M., Liao, F., Lin, X., Hsiao, H. M., and 9 others 2019. “Mitochondrial Damage-associated Molecular Patterns Released by Lung Transplants are Associated with Primary Graft Dysfunction,” American Journal of Transplantation 19(5): 1464-1477). Accordingly, initiating switch transplantation immediately upon death avoids an increase in donor blood factors inimical to success.

Inapparent upon gross inspection, this microscopic level of degeneration will not be avoided much less reversed by chilled storage, and whether ‘normal’ reperfusion and revival of living cells will allow clearance of this microdebris likely depends upon its prevalence and the relative proportion of cells capable of recovering to initiate its clear up, which argues in favor of hematopoietic cell therapy. Once this has been allowed to happen, the organ has already become severely degraded for transplantation, already broken down before the final insult-placement in an alien, if allogeneic milieu. Often the donor is discovered having already died and a long waiting list forces the staff to make the most of the organs available. Then, improvements in preservation strategies are of incontestable value. Ordinarily, when a prospective donor dies, blood is cut off from all of the donor's organs.

While anatomically distinguishable, solid organs are not properly viewed as separate from the rest of the body. Ex vivo perfusion machines facilitate targeted therapy of the graft organ through the crude method of having excised and thus isolated the organ from its physiological context. When intact in its normal situation, the chemical and mechanical interactions of the organ with the rest of the body are multifarious and many, completely interconnected as would interact with the therapy in ways numerous and complex. To then transplant the organ resituates it in the equivalent milieu with no reduction in the complexity of contextual function but with the added complication of immunological alteration.

In contrast to therapy with a perfusion machine, metered compound vascular bypass, or sudden switched-organ transplantation allows the incorporation of graft organ-targeted therapy while the organ remains in situ within the donor sustained on life support and during transfer from the circulatory system of the donor to that of the recipient. It accomplishes this, moreover, with no breach in bodily connections throughout this unprecedented transition—that is, with no interruption in perfusion, respiration, or other life functions despite having been resituated from one body to another. By continuously sustaining all functions of the graft organ, first while remaining in the donor, then, albeit partially denervated (Choudhury, M. 2017. “Post-cardiac Transplant Recipient: Implications for Anaesthesia,” Indian Journal of Anaesthesia 61(9): 768-774) but with intact vascular connections in the recipient, the considerable trauma associated with death, organ preservation, and conventional transplantation are avoided.

In addition to eliminating numerous insults associated with conventional transplantation that contribute to graft failure, metered compound vascular bypass, or sudden switched-organ transplantation inherently accomplishes a measure of immune tolerance induction in the interval during which the organ is transferred from the donor to the recipient. Furthermore, because the accessory channels of the diversion chute servovalves used to connect the native organ of the recipient to the graft organ while it remains in vivo in the donor allow the direct targeting of agents to either or both hearts during the transfer, the application of concurrent therapy, pharmaceutical or genetic can be used to restore the graft organ while it is being resituated in the recipient.

The immune tolerance induction accomplished concurrently with transfer to the recipient of the graft organ may allow a reduction in the dosages of tolerance induction drugs such as antithymocyte globulin and dendritic cells, which risk numerous disturbing side effects, to include fever, chills, aphthous ulcers, malaise, tachycardia, and dyspnea. During metered compound vascular transplantation administered by an implanted prosthetic disorder response system, the master control microprocessor prescription-program receives inputs from strategically positioned sensor implants.

When these signal adverse reactions to the regimen in progress, the master controller reduces or stops release of the culprit substance release in accordance with its prescription-program. Such an adjustment can be applied at the drug reservoir outlet pump stopcock or by adjusting the rate of servomotor advancement. Characterization thus is not meant to discount the relegation of such a function to extracorporeal apparatus, especially in complex comorbidity where the number of implanted components would cause discomfort.

Basiliximab risks tremor, fever, chills, diarrhea (steatorrhea), and vomiting (Phillips, B. E., Garciafigueroa, Y., Engman, C., Trucco, M., and Giannoukakis, N. 2019. “Tolerogenic Dendritic Cells and T-Regulatory Cells at the Clinical Trials Crossroad for the Treatment of Autoimmune Disease; Emphasis on Type 1 Diabetes Therapy,” Online, Frontiers in Immunology (Lausanne, Switzerland) 10:148; Thomson, A. W., Metes, D. M., Ezzelarab, M. B., and Raich-Regue, D. 2019. “Regulatory Dendritic Cells for Human Organ Transplantation,” Transplantation Reviews (Orlando, Florida) 33(3): 130-136; Obregon, C., Kumar, R., Pascual. M. A., Vassalli, G., and Golshayan, D. 2017. “Update on Dendritic Cell-induced Immunological and Clinical Tolerance,” Online, Frontiers in Immunology (Lausanne, Switzerland) 8:1514; Waldmann, H., Howie, D., and Cobbold, S. 2016. “Induction of Immunological Tolerance as a Therapeutic Procedure,” Microbiology Spectrum 4(4); Silva Pde. M., Bier, J., Paiatto, L. N., Galdino Albuquerque, C., Lopes Souza, C., and 3 others 2015. “Tolerogenic Dendritic Cells on Transplantation: Immunotherapy Based on Second Signal Blockage,” Journal of Immunology Research 2015:856707; Aliabadi, A., Grommer, M., Cochrane, A., Salameh, O., and Zuckermann, A. 2013. “Induction Therapy in Heart Transplantation: Where are we Now?,” Transplant International 26(7): 684-695; Golshayan, D. and Pascual. M. 2008. “Tolerance-inducing Immunosuppressant Strategies in clinical Transplantation: An Overview,” Drugs (Auckland, New Zealand) 68(15): 2113-2130; Pihusch, R., Holler, E., Muhlbayer, D., Gohring. P., Stotzer. O., and 3 others 2002. “The Impact of Antithymocyte Globulin on Short-term Toxicity after Allogeneic Stem Cell Transplantation,” Bone Marrow Transplantation 30(6): 347-354; Kobayashi, R., Kaneda, M., Watanabe, N., Iguchi, A., Cho. Y., and 5 others 1999. “Adverse Effects of Anti-thymocyte Globulin/Anti-lymphocyte Globulin Therapy,” (in Japanese with abstract at Pubmed), Rinsho Ketsueki [Japanese Journal of Clinical Hematology] 140(7): 531-535).

While drugs, regulatory T cells, donor bone marrow, and stem cells can also be integrated into the process, organ transplantation using the metered switch method should accomplish a measure of blood-induced immune tolerance induction as an inherent byproduct of the reciprocal cross-circulation that mediates transfer of the organ from the donor to the recipient (see, for example, Gupta, P. K., McIntosh, C. M., Chong, A. S., and Alegre, M. L. 2019. “The Pursuit of Transplantation Tolerance: New Mechanistic Insights,” Cellular and Molecular Immunology (Beijing, China) 16(4): 324-333: Thangavelu, G. and Blazar, B. R. 2019. “Achievement of Tolerance Induction to Prevent Acute Graft-vs.-Host Disease,” Online, Frontiers in Immunology (Lausanne, Switzerland) 10:309; Miller, M. L., Chong, A. S., and Alegre, M. L. 2017. “Fifty Shades of Tolerance: Beyond a Binary Tolerant/Non-Tolerant Paradigm,” Current Transplantation Reports (Cham, Switzerland) 4(4): 262-269; Zuber, J. and Sykes, M. 2017. “Mechanisms of Mixed Chimerism-based Transplant Tolerance,” Trends in Immunology 38(11): 829-843; additional references follow in this section).

This factor likely improves the prospects for kidneys and livers more than lungs and hearts, the latter tending more to resist immune tolerance induction. However, with the incorporation of gene therapy (Bishawi, M., Roan, J. N., Milano, C. A., Daneshmand, M. A., Schroder, J. N., and 10 others 2019. “A Normothermic ex Vivo Organ Perfusion Delivery Method for Cardiac Transplantation Gene Therapy,” Nature Research Scientific Reports 9(1): 8029) both to augment microchimerization and aid restoration, and with the further addition of the following agents and those specified in sections to follow, the prospects for lungs and hearts should also improve (see also Bulaklak, K., and Gersbach, C. A. 2020. “The Once and Future Gene Therapy,” Online, Nature Communications (London, England) 11:5820).

Means for acclimating the recipient and donor to the cells, tissues, and genes of one another are effective in proportion to the time of exposure and/or cross exposure, so that as well as to continue during reciprocal cross-circulation, and along with immune tolerance-inducing medication, such treatment should be initiated as soon before actual organ transfer as possible. The risks of rejection and of infection during such an exchange of bodily materials due to the administration of immunosuppressives is addressed above in this section. Administered by the automatic disorder response system, rejection and infection sensors specified above in the section can signal the need to slow, stop, and/or treat such adverse eventualities.

Numerous references would appropriately have been included in more than one section. Additives to the perfusate using conventional means for graft organ preservation such as ex vivo perfusion can also pertain to the addition of such an agent into the bloodstream shared by the donor and recipient which mediates the metered switch. The perioperative introduction of immune tolerance induction drugs and/or steroids, are example.

a. Chimerizing hematopoietic or mesenchymal stem cell therapy (see, for example, Trino, S., Zoppoli, P., Carella, A. M., Laurenzana, I., Weisz, A., and 9 others 2019. “DNA Methylation Dynamic of Bone Marrow Hematopoietic Stem Cells after Allogeneic Transplantation,” Stem Cell Research and Therapy 10(1): 138; Ichim. T. E., O'Heeron, P., and Kesari, S. 2018. “Fibroblasts as a Practical Alternative to Mesenchymal Stem Cells,” Online, BioMed Central Journal of Translational Medicine 16(1): 212; Mahr. B., Granofszky, N., Muckenhuber, M., and Wekerle, T. 2017. “Transplantation Tolerance through Hematopoietic Chimerism: Progress and Challenges for Clinical Translation” Online, Frontiers in Immunology (Lausanne, Switzerland) 8:1762; Singleton, A., Khong, D., Chin, L. Y., Mukundan, S., Li, M., and Parekkadan, B. 2017. “An Engineered Biomarker System to Monitor and Modulate Immune Clearance of Cell Therapies,” Cytotherapy 19(12): 1537-1545; Yolcu, E. S., Shirwan, H., and Askenasy, N. 2017. “Mechanisms of Tolerance Induction by Hematopoietic Chimerism: The Immune Perspective,” Online, Stem Cells Translational Medicine 6(3): 700-712; Reinders, M. E. and Hoogduijn, M. J. 2014. “NK [natural killer] Cells and MSCs [mesenchymal stem cells]: Possible Implications for MSC Therapy in Renal Transplantation,” Online, Journal of Stem Cell Research and Therapy 4(2): 1000166; Kawai. T. and Sachs, D. H. 2013. “Tolerance Induction: Hematopoietic Chimerism,” Current Opinion in Organ Transplantation 18(4): 402-407; Miura, Y., Yoshioka, S., Yao, H., Takaori-Kondo, A., Maekawa, T., and Ichinohe, T. 2013. “Chimerism of Bone Marrow Mesenchymal Stem/Stromal Cells in Allogeneic Hematopoietic Cell Transplantation: Is It Clinically Relevant?,” Chimerism (Austin, Texas) 4(3): 78-83; Pilat, N., Hock, K., and Wekerle, T. 2012. “Mixed Chimerism through Donor Bone Marrow Transplantation: A Tolerogenic Cell Therapy for Application in Organ Transplantation,” Current Opinion in Organ Transplantation 17(1): 63-70; Hoogduijn, M. J., Popp, F. C., Grohnert. A., Crop. M. J., van Rhijn, and 25 others 2010. “Advancement of Mesenchymal Stem Cell Therapy in Solid Organ Transplantation (MISOT),” Transplantation 90(2): 124-126; Khan, F., Agarwal, A., and Agrawal, S. 2004. “Significance of Chimerism in Hematopoietic Stem Cell Transplantation: New Variations on an Old Theme,” Bone Marrow Transplantation 34(1): 1-12; Nikolic. B. and Sykes, M. 1997. “Mixed Hematopoietic Chimerism and Transplantation Toleance,” Immunologie Research 16(3): 217-228; additional references cited below in section 2b(1)(b)(b5), entitled Heterotopic Double Heart Metered Compound Vascular Bypass, or Switched-organ Transplantation [and so on]).

b. Restorative stem cell therapy, to restore a donor heart if necessary (see, for example, Hong, I. S. 2022. Enhancing Stem Cell-based Therapeutic Potential b Combining Various Bioengineering Technologies,” Online, Frontiers in Cell and Developmental Biology 10:901661; Johnstone, B. H., Messner, F., Brandacher, G., and Woods, E. J. 2021. “A Large-scale Bank of Organ Donor Bone Marrow and Matched Mesenchymal Stem Cells for Promoting Immunomodulation and Transplant Tolerance,” Online, Frontiers in Immunology (Lausanne, Switzerland) 12:622604; Yao, X., Ma. Y., Zhou, W., Liao. Y., Jiang, Z., and 7 others 2021. “In-cytoplasm Mitochondrial Transplantation for Mesenchymal Stem Cells Engineering and Tissue Regeneration,” Online, Bioengineering and Translational Medicine 7(1): e10250; Zhang, T., Xu, Q., Huang. T., Ling. D., and Gao. J. 2020. “New Insights into Biocompatible Iron Oxide Nanoparticles: A Potential Booster of Gene Delivery to Stem Cells,” Nano Micro Small 16(37): e20011588; Bishawi, M., Roan, J. N., Milano, C. A., Daneshmand, M. A., Schroder, J. N., and 10 others 2019. Op cit. in this section; Zakrzewski, W., Dobrzyński, M., Szymonowicz, M., and Rybak, Z. 2019. “Stem Cells: Past, Present, and Future,” Online, BioMed Central Stem Cell Research and Therapy (London, England) 10(1): 68; Smits, A. M., Dronkers, E., and Goumans, M. J. 2018. “The Epicardium as a Source of Multipotent Adult Cardiac Progenitor Cells: Their Origin, Role and Fate,” Pharmacological Research 127:129-140; Xu Q., Zhang. T., Wang, Q., Jiang, X, Li, A., and 6 others 2018. “Uniformly Sized Iron Oxide Nanoparticles for Efficient Gene Delivery to Mesenchymal Stem Cells,” International Journal of Pharmaceutics 552(1-2): 443-452; Mahla. R. S. 2016. “Stem Cells Applications in Regenerative Medicine and Disease Therapeutics,” International Journal of Cell Biology 2016:6940283; Singh V. K., Kalsan, M., Kumar, N., Saini, A., and Chandra, R. 2015. “Induced Pluripotent Stem Cells: Applications in Regenerative Medicine, Disease Modeling, and Drug Discovery,” Online, Frontiers in Cell and Developmental Biology (Lausanne, Switzerland) 3:2; Singh, V. K., Kumar, N., Kalsan, M., Saini, A., and Chandra, R. 2015. “Mechanism of Induction: Induced Pluripotent Stem Cells (iPSCs),” Journal of Stem Cells 10(1): 43-62).

c. Gene silencing. Another approach to maximize tolerance induction before sudden compound vascular bypass, or sudden switched-organ transplantation and during and after metered compound vascular bypass, or sudden switched-organ transplantation is gene silencing with small interfering RNA (siRNA). That the conventional object is to reduce ischemia-reperfusion injury following organ storage, and this has already been eliminated in switch transplantation, treatment thus may also improve the odds for avoiding the development of post-transplantation heart vasculopathy (Brüggenwirth, I. M. A. and Martins, P. N. 2019. “RNA Interference Therapeutics in Organ Transplantation: The Dawn of a New Era,” American Journal of Transplantation November 4; Thijssen, M. F., Brüggenwirth, I. M. A., Gillooly, A., Khvorova, A., Kowalik, T. F., and Martins, P. N. 2019. “Gene Silencing with siRNA [small interfering RNA] (RNA Interference): A New Therapeutic Option during ex Vivo Machine Liver Perfusion Preservation,” Liver Transplantation 25(1): 140-151; Wei, J., Chen, S., Xue. S., Zhu, Q., Liu, S., and 3 others 2017. “Blockade of Inflammation and Apoptosis Pathways by siRNA Prolongs Cold Preservation Time and Protects Donor Hearts in a Porcine Model,” Molecular Therapy. Nucleic Acids 9:428-439; Zheng, X., Zang, G., Jiang, J., He, W., Johnston, N. J., and 8 others 2016. “Attenuating Ischemia-Reperfusion Injury in Kidney Transplantation by Perfusing Donor Organs with siRNA Cocktail Solution,” Transplantation 100(4): 743-752: Zheng. X., Lian, D., Wong. A., Bygrave. M., Ichim, T. E., and 8 others 2009. “Novel Small Interfering RNA-containing Solution Protecting Donor Organs in Heart Transplantation,” Circulation 120(12): 1099-1108).

In compound vascular bypass transplantation, ischemia is eliminated and therewith ischemia-reperfusion injury, but not a scientific interest in pursuing its understanding. Intriguingly, post transplantation function is impaired following prolonged cold storage, but continued perfusion appears to resolve functional impairment (Lee, C. M., Carter, J. T., Randall, H. B., Hiose, R., Stock, P. G., and 4 others 2000. “The Effect of Age and Prolonged Cold Ischemia Times on the National Allocation of Cadaveric Renal Allografts,” Journal of Surgical Research 91(1): 83-88; (see also Mergental, H., Stephenson, B. T. F., Laing, R. W., Kirkham, A. J., Neil, D. A. H., and 11 others 2018. “Development of Clinical Criteria for Functional Assessment to Predict Primary Nonfunction of High-risk Livers Using Normothermic Machine Perfusion,” Liver Transplantation 24(10): 1453-1469; Nasralla, D., Coussios, C. C., Mergental. H., Akhtar, M. Z., Butler, A. J., and 25 others 2018. “A Randomized Trial of Normothermic Preservation in Liver Transplantation,” Nature 557(7703): 50-56; Schofield, Z. V., Woodruff. T. M., Halai. R., Wu. M. C., and Cooper, M. A. 2013. “Neutrophils-A Key Component of Ischemia-Reperfusion Injury,” Shock 40(6): 463-470; Uygun, K., Tolboom, H., Izamis, M.-L., Uygun, B., Sharma, N., and 5 others 2010. “Diluted Blood Reperfusion as a Model for Transplantation of Ischemic Rat Livers: Alanine Aminotransferase is a Direct Indicator of Viability,” Transplantation Proceedings 42(7): 2463-2467; Watson, C. J., Wells, A. C., Roberts. R. J., Akoh, J. A., Friend, P. J., and 5 others 2010. “Cold Machine Perfusion versus Static Cold Storage of Kidneys Donated after Cardiac Death: A UK Multicenter Randomized Controlled Trial,” American Journal of Transplantation 10(9): 1991-1999; Imber, C. J., St. Peter, S. D., Lopez de Cenarruzabeitia, I., Pigott, D., James, T., and 6 others 2002. “Advantages of Normothermic Perfusion over Cold Storage in Liver Preservation,” Transplantation 73(5): 701-709)

What is true for a heart transplant is likely no less true for other organ transplants: the avoidance of oxygen deprivation and an adverse immune response before and during transplantation, which switch transplantation provides, and controlling comorbidities after transplantation, which an implanted disorder response system provides, will improve the prospects for longer eventual survival of any organ transplant (see, for example, Braun, A. T. and Merlo, C. A. 2011. “Cystic Fibrosis Lung Transplantation,” Current Opinion in Pulmonary Medicine 17(6): 467-472).

The complete avoidance of preservation, hence, preservation-associated injury, obtained by directly transferring the graft organ from the circulatory system of the donor into that of the recipient materially if not entirely eliminates breakdown products of injured and dying donor cells the result of prolonged ischemia during storage, such cells a prime target of recipient immune cells. Moreover, that the donor is supported in oxygen delivery while still alive means that fewer such cells result from an hypoxic state of the donor prior to death.

Pretransplantation cellular breakdown contributes to graft rejection both endothelial to include even late-term cardiac allograft vasculopathy and “In the parenchyma, complement fixation initiates the complement cascade, culminating in the formation of a membrane attack complex,” and this breakdown in the cell membranes opens the cells to osmotic injury (Rohrer, R. J. 2002. Basic Immunology for Surgeons,” in O'Leary, J. P. (ed.), The Physiologic Basis of Surgery, Philadelphia, Pennsylvania: Lippincott Williams and Wilkins).

d. Supplemental hydrogen sulfide (sulphide)(Lobb. I., Jiang, J., Lian, D., Liu, W., Haig. A., and 5 others 2017. “Hydrogen Sulfide Protects Renal Grafts against Prolonged Cold Ischemia-Reperfusion Injury via Specific Mitochondrial Actions,” American Journal of Transplantation 17(2): 341-352; Lobb, I., Davison, M., Carter, D., Liu, W., Haig, A., Gunaratnam, L., and Sener, A. 2015. “Hydrogen Sulfide Treatment Mitigates Renal Allograft Ischemia-Reperfusion Injury during Cold Storage and Improves Early Transplant Kidney Function and Survival Following Allogeneic Renal Transplantation” Journal of Urology (Philadelphia, Pennsylvania) 194(6): 1806-1815; Lobb, I., Mok. A., Lan, Z., Liu, W., Garcia, B., and Sener, A. 2012. “Supplemental Hydrogen Sulphide Protects Transplant Kidney Function and Prolongs Recipient Survival after Prolonged Cold Ischaemia-reperfusion Injury by Mitigating Renal Graft Apoptosis and Inflammation,” British Journal of Urology International 110(11 Part C): E1187-E1195).

e. Gene therapy. Persistent problems with gene therapy include transience, transfection virus toxicity, gene mistargeting due to pleiotropy, affecting not just the somatic but the germ line, tumorigenesis, leukemogenesis, and exorbitant cost (see, for example, Ertl. H. C. J. 2023. “Mitigating Serious Adverse Events in Gene Therapy with AAV [adeno-associated viral] Vectors: Vector Dose and Immunosuppression,” Drugs 83(4): 287-298; Mendola Pla, M., Chiang. Y., Roki. A., Wang, C., Lee, F. H., and 12 others 2023. “Ex vivo Gene Delivery to Porcine Cardiac Allografts Using a Myocardial-enhanced Adeno-associated Viral Vector,” Human Gene Therapy March 16 (in press); Ertl. H. C. J. 2022. “Immunogenicity and Toxicity of AAV Gene Therapy,” Online, Frontiers in Immunology (Lausanne, Switzerland) 13:975803; Gao, Q., DeLaura, I. F., Anwar, I. J., Kesseli, S., Kahan, R., and 4 others 2022. “Gene Therapy: Will the Promise of Optimizing Lung Allogafts Become Reality?,” Online, Frontiers in Immunology (Lausanne, Switzerland) 13:931524; Ohm, B. and Jungraithmayr, W. B. 2022. “Cell Immunity in Lung Transplant Rejection—Effector Mechanisms and Therapeutic Implications,” Online, Frontiers in Immunology (Lausanne, Switzerland) 13:845867; Pia, M. M., Evans, A., Lezberg, P., and Bowles, D. E. 2022. “Ex vivo Delivery of Viral Vectors by Organ Perfusion for Cardiac Transplantation Gene Therapy,” Cardiac Gene Therapy Methods and Protocols 2573:249-259; Ertl. H. C. J. 2021. “T Cell-mediated Immune Responses to AAV [adeno-associated virus] and AAV [adeno-associated viral] Vectors,” Online, Frontiers in Immunology (Lausanne, Switzerland) 12:666666; Newby, G. A. and Liu, D. R. 2021. “In vivo Somatic Cell Base Editing and Prime Editing,” Online, Molecular Therapy 29(11): 3107-3124; Papanikolaou, E. and Bosio, A. 2021. “The Promise and the Hope of Gene Therapy,” Online, Frontiers in Genome Editing 3:618346; Zittersteijn, H. A., Goncalves, M. A. F. V., and Hoeben, R. C. 2021. “A Primer to Gene Therapy: Progress, Prospets, and Problems,” Online, Journal of Inherited Metabolic Disease 44(1): 54-71; Bishawi, M., Roan, J. N., Milano, C. A., Daneshmand, M. A., Schroder, J. N., and 10 others 2019. “A Normothermie Ex vivo Organ Perfusion Delivery Method for Cardiac Transplantation Gene Therapy,” Nature Research Scientific Reports 9(1): 8029; Moscoso, C. G. and Steer, C. J. 2019. “Liver Targeted Gene Therapy: Insights into Emerging Therapies,” Drug Discovery Today Technologies 34:9-19; Goncalves, G. A. R. and Palva, R. M. A. 2017. “Gene Therapy: Advances, Challenges, and Perspectives,” Online, Einstein (São Paulo, Brazil) 15(3): 369-375; Li, J., Qi, G., Tu, G., Yang, C., and Rong. R. 2017. “Gene Therapy in Kidney Transplantation: Evidence of Efficacy and Future Directions,” Current Gene Therapy 17(6): 434-441; Mayo Clinic 2017. “Gene Therapy,” Online, mayoclinic.org; Rahim, F. and Ebrahimi, A, 2014. “Gene Therapy Modalities in Lung Transplantation,” Transplant Immunology 31(3): 165-172; von Kalle, C., Deichmann, A., and Schmidt, M. 2014. “Vector Integration and Tumorigenesis,” Online, Human Gene Therapy 25(6): 475-481; Yeung, J. C., Wagnetz, D., Cypel, M., Rubacha. M., Kolke. T., and 6 others 2012. “Ex vivo Adenoviral Vector Gene Delivery Results in Decreased Vector-associated Inflammation Pre- and Post-lung Transplantation in the Pig,” Online, Molecular Therapy 20(6): 1204-1211; Sandovici, M., Deelman, L. E., Benigni, A, and Henning, R. H. 2010. “Towards Graft-specific Immune Suppression: Gene Therapy of the Transplanted Kidney,” Advanced Drug Delivery Reviews 62(14): 1358-1368; Vassalli, G., Roehrich, M. E., Vogt. P., Pedrazzini, G. B., Siclari, F., Moccetti, T., and von Segesser, L. K. 2009. “Modalities and Future Prospects of Gene Therapy in Heart Transplantation” Online, European Journal of Cardiothoracic Surgery 35(6): 1036-1044; Sandovici, M., Deelman, L. E., de Zeeuw, D., van Goor, H., and Henning, R. H. 2008. “Immune Modulation and Graft Protection by Gene Therapy in Kidney Transplantation,” European Journal of Pharmacology 585(2-3): 261-269; Dunbar, C. E. 2007. “The Yin and the Yang of Stem Cell Gene Therapy: Insights into Hematopoiesis, Leukemogenesis, and Gene Therapy Safety,” Online, Hematology American Society of Hematology Education Program 2007:460-465: Moore, D. J., Markmann, J. F., and Deng. S. 2006. “Avenues for Immunomodulation and Graft Protection by Gene Therapy in Transplantation,” Online, Transplant International 19(6): 435-445; Bagley, J. and Iacomini, J. 2003. “Gene Therapy Progress and Prospects: Gene Therapy in Organ Transplantation,” Gene Therapy 10(8): 605-611; Wood, K. J. and Prior. T. G. 2001. “Gene Therapy in Transplantation,” Current Opinion in Molecular Therapy 3(4): 390-398). Gene therapy is also addressed above in this section.

The main objective in tolerance induction is to reduce if not eliminate the need for lifelong immunosuppression. Another factor in switch transplantation, the use of an implanted automatic disorder response system to directly pipeline-target immunosuppressants to the transplant, sparing exposure of the rest of the body, can compensate for a shortcoming in tolerance induction and increased susceptibility to infection (Tonsho, M., Michel, S., Ahmed, Z., Alessandrini, A., and Madsen, J. C. 2014. “Heart Transplantation: Challenges Facing the Field,” Cold Spring Harbor Perspectives in Medicine 4(5). pii: a015636) and instigation of cancer (Manzia, T. M., Angelico, R., Gazia, C., Lenci, I., Milana, M., and 6 others 2019. “De Novo Malignancies after Liver Transplantation: The Effect of Immunosuppression-Personal Data and Review of Literature,” World Journal of Gastroenterology 25(35): 5356-5375; Merchea. A., Shahjehan, F., Croome, K. P., Cochuyt. J. J., Li, Z., Colibaseanu, D. T., and Kasi. P. M. 2019. “Colorectal Cancer Characteristics and Outcomes after Solid Organ Transplantation,” Journal of Oncology 2019:5796108; Wang, H. E., Liao, Y. C., Hu. J. M., Wu. W. C., Chou, W. Y., and 6 others 2019. “Correlation between Kidney Transplantation and Colorectal Cancer in Hemodialysis Patients: A Nationwide, Retrospective, Population-based Cohort Study,” Online, BioMed Cancer 19(1): 1120; Acuna, S. A. 2018. “Etiology of Increased Cancer Incidence after Solid Organ Transplantation,” Transplantation Reviews (Orlando, Florida) 2018 32(4): 218-224; Park, M. J., Roh, J. L., Choi, S. H., Nam, S. Y., Kim, S. Y., and Lee, Y. S. 2018. “De Novo Head and Neck Cancer Arising in Solid Organ Transplantation Recipients: The Asan Medical Center Experience,” Auris, Nasus, Larynx [Ear, Nose, Throat] 45(4): 838-845; Kitahara, C. M., Yanik, E. L., Ladenson, P. W., Hernandez, B. Y., Lynch, C. F., Pawlish, K. S., and Engels, E. A. 2017. “Risk of Thyroid Cancer among Solid Organ Transplant Recipients,” American Journal of Transplantation 17(11): 2911-2921; Kluijfhout, W. P., Drake, F. T., Pasternak, J. D., Beninato, T., Mitmaker, E. J., and 5 others 2017. “De Novo Thyroid Cancer following Solid Organ Transplantation-A 25-year Experience at a High-volume Institution with a Review of the Literature,” Journal of Surgical Oncology 115(2): 105-108; Mouchli. M. A., Singh, S., Loftus, E. V. Jr., Boardman, L., Talwalkar, J., and 6 others 2017. “Risk Factors and Outcomes of de Novo Cancers (Excluding Nonmelanoma Skin Cancer) after Liver Transplantation for Primary Sclerosing Cholangitis,” Transplantation 101(8): 1859-1866; Mukthinuthalapati, P. K., Gotur, R., and Ghabril, M. 2016. “Incidence, Risk Factors and Outcomes of de Novo Malignancies Post Liver Transplantation,” World Journal of Hepatology 8(12): 533-544; Penn, I. 1991. “Cancer in the Immunosuppressed Organ Recipient,” Transplantation Proceedings 23(2): 1771-1772; Penn, I. 1986. “Cancer is a Complication of Severe Immunosuppression,” Surgery, Gynecology, and Obstetrics 162(6): 603-610). Addressed below in this section, another implicated sequela, cardiac allograft vasculopathy, responsible for half of failed heart transplants, is likely due, at least in part, to ischemia during preservation.

Both sudden and metered compound vascular bypass, or sudden switched-organ heart transplantation are extracardiac, eliminating the extensive dissection of both donor and recipient hearts which is a key factor in conventional methods of heart transplantation and the basis for the risk of problem bleeding. While the benefits of avoiding surgical trauma in the transition from the original biatrial to the bicaval technique are well documented (see, for example, Tonsho, M., Michel, S., Ahmed, Z., Alessandrini, A., and Madsen, J. C. 2014, Op cit.), the additional benefits in continuing the pattern of retreating from the heart entirely during heart transplantation has no precedent in human medical practice, and while animal experiments are documented, no such operations in man appear to have been recorded in the literature. The lack of immunosuppressives would have assured failure in any event, and the resultant immunosuppression necessarily systemic, the recipient left susceptible to any infectious disease would inevitably have died anyway.

That neither donor nor recipient hearts are entered eliminates the risks associated with cardioplegia, cross-clamping, general anesthesia, cardiopulmonary bypass, ischemia-reperfusion injury, and infection, should reduce the incidence of post-transplantation vasculopathy, and as addressed herein, given that immunosuppressants and tolerance induction drugs are primarily pipeline-targeted to the transplant, should reduce de novo post-transplantation cancer—that is, reduce or eliminate the complications known to limit transplant and recipient longevity. The elimination of cardiopulmonary bypass may be more significant than just the avoidance of postoperative cognitive deficits (Imamura, T., Kinugawa, K., Fujino, T., Inaba, T., Maki, H., and 5 others 2014. “Recipients with Shorter Cardiopulmonary Bypass Time Achieve Improvement of Parasympathetic Reinnervation within 6 Months after Heart Transplantation,” International Heart Journal 55(5): 440-444).

The avoidance of these complications depends upon the avoidance of the biochemical upheaval associated with death, the abrupt loss of perfusion, anoxia, organ harvesting, preservation, and insertion into an alien milieu. Sustainment past brain death and the switch method allow these eventualities, ruinous soon and/or posing the risk of vasculopathy and/or cancer. Later to be avoided, and the switch method is dependent upon access to vascular valves and servovalves.

The metered switch method effectively achieves intrinsic and concurrent in vivo graft organ/recipient immunosuppression, immune tolerance induction, and a measure of cross-microchimerization through reciprocal cross-circulation during organ transfer as well as the transfer of donor-derived passenger leukocytes to the recipient, and to the cross-chimerization inherent in reciprocal cross-circulation, allows the incorporation of gene and stem cell therapy for both increased tolerance induction and graft organ restoration superior to that obtained with ex vivo perfusion with its numerous detractions in exchange for this one advantage.

Always a ‘two-edged sword,’ the introduction of allogeneic protein can provoke an immune reaction, even intense, or effect a measure of immune tolerance, even chimerization, thus facilitating the acceptance of an allogeneic graft organ. As with immunosuppression by any means, an object is to blunt if not avoid an initial reaction of outright and immediate recoil and educe instead an initial accommodation. In mollifying an adverse reaction, eventual acceptance should be facilitated, and in a heart transplant, the odds favoring eventual failure due to allograft vasculopathy should be reduced.

In a metered compound vascular bypass, or sudden switched-organ transplantation, the transfer of the graft organ from the donor to the recipient is initiated by admitting a minute volume of recipient blood into the circulation of the donor. Implanted sensors detect signs or biomarkers of rejection by the recipient or an adverse immune reaction by the donor that would negatively affect the other organs. If none, then the process is continued, very gradually increasing the relative proportion of recipient over donor blood until the blood circulating through both is half that of the other. At any point in the process, the valves can are automatically adjusted to cut off entirely, or gradually reduce, or increase the relative volume of blood exchanged.

An adverse reaction is responded to by infusing immunosuppressants, and once suppressed, the process is continued. The treatment of problem bleeding if any following the procedure is dealt with by conventional means (Kunadian, V., Zorkun, C., Gibson, W. J., Nethala, N., Harrigan, C., and 10 others 2009. “Transfusion Associated Microchimerism: A Heretofore Little-recognized Complication following Transfusion,” Journal of Thrombosis and Thrombolysis 27(1): 57-67).

Transfusion associated microchimerization has been long recognized (see, for example, United States National Library of Medicine Clinical Trials.gov. 2017. “Clinical Study of Microchimerism and cfDNA [circulating free DNA] as Biomarkers for Acute Rejection after Organ Transplantation,” Online at clinicaltrials.gov/ct2/show/NCT03255265; Ueta, H., Kitazawa, Y., Sawanobori, Y., Ueno, T., Ueha, S., Matsushima, K., and Matsuno, K. 2018. “Single Blood Transfusion Induces the Production of Donor-specific Alloantibodies and Regulatory T Cells Mainly in the Spleen,” International Immunology 30(2): 53-67; Bloch, E. M., Jackman, R. P., Lee, T. H., and Busch, M. P. 2013. “Transfusion-associated Microchimerism: The Hybrid Within,” Transfusion Medicine Reviews 27(1): 10-20; Jovičić, S., Ležaić, V., Simonović, R., and Djukanovic, L. 2011. “Beneficial Effects of Donor-specific Transfusion on Renal Allograft Outcome,” Clinical Transplantation 25(2): 317-324; Dunne, J. R., Lee, T. H., Burns, C., Cardo, L. J., Curry, K., and Busch, M. P. 2008. “Transfusion-associated Microchimerism in Combat Casualties,” Joumal of Trauma 64(2 Supplement): S92-S98; Wood, K. J. 2003. “Passenger Leukocytes and Microchimerism: What Role in Tolerance Induction?,” Transplantation 75(9 Supplement): 17S-20S), and the cross-chimerization inherent in reciprocal cross-circulation allows the incorporation of gene and stem cell therapy for both increased tolerance induction and graft organ restoration superior to that obtained with ex vivo perfusion with its numerous detractions in exchange for this one advantage.

Rather than isolated from the rest of the body in a perfusion machine, the organ remains in its physiological situation, and following this procedure, the implanted automatic response system can continue this treatment. If uptake of the agent by the target organ is incomplete and the agent would best be kept from nontargeted tissue, the accessory channels in the valve-jackets at the venous drainage of the organ can be used to release a reversal agent or if necessary, the agent can be formulated for magnetic extraction. Radioactive agents can be targeted to the organ and if necessary, extracted, through shielded lines and connectors.

As shown in U.S. Pat. No. 11,759,186, entitled Ductus Side-entry Jackets and Prosthetic Disorder Response Systems, such a side-entry jacket modified to serve as a vascular valve can incorporate a layer of permanent, preferably neodymium, magnets magnetized to project an attractive field radially toward the central axis of the substrate ductus, one object in incorporating a magnetized layer being to facilitate the uptake of superparamagnetic micro- and nanoparticle carrier bound drugs into the intima or comparable layer in ductus other than an artery. Such valves can also incorporate different type sensors to detect various physiological indicia, the positioning of these dependent upon the index to be measured.

The cutoff from oxygen, nutrients, electrolytes, enzymes, hormones, neurochemical and nervous connections, and metabolites known and possibly remaining to be discovered initiates a cascade of degenerative changes that will culminate in autophagy. The graft organ is transplanted not just with its allogeneic genome and its individual differences but also with immune system-produced irreversible degradation products, dead cells, cells losing or having lost membrane permeability, and likely, other cellular level toxins associated with the death of the organism and its cells. The degrees to which post-death measures can revive this tissue and to which harvesting adds further damage depend upon the time elapsed since death.

Recipient-donor cross-chimerization central in metered compound vascular bypass, or switched-organ transplantation, references thereto appear in several sections herein, to include 2a(3) and 2b(1)(b). It warrants emphasis that, acknowledging the uncontrollability of donor organ procurement in most cases, whenever possible, the direct interconnection of the circulatory systems of recipient and donor through the native and graft organs prior to harvesting is fundamentally superior to any alternative, whether machine perfusion, cold storage, special organ transport support machines, normothermal, cryothermal, or any other alternative.

When refrigerated and/or preserved in a chilled static or flowing perfusate, the deterioration of the graft organ can be slowed down if not stabilized at the level to which it had progressed, and unless the time of this stasis is relatively brief and aggressive measures are applied and succeed, it is in this condition that the graft organ is placed in a recipient. Ex vivo perfusion machines afford an opportunity to apply therapy and ameliorate but not eliminate the problem.

Metered compound vascular bypass, or sudden switched-organ transplantation can be continued long enough to achieve a measure of immune tolerance induction as well as meet or exceed the repair obtained using ex vivo lung perfusion (see, for example, Chen, Y. B., Elias, N., Heher, E., McCune, J. S., Collier, K., and 14 others 2019. “Haploidentical Hematopoietic Cell and Kidney Transplantation for Hematological Malignancies and End-stage Renal Failure,” Blood 134(2): 211-215; Ezzelarab, M. B., Perez-Gutierrez, A., Humar, A., Wijkstrom, M., Zahorchak, A. F., and 5 others 2019. “Preliminary Assessment of the Feasibility of Autologous Myeloid-derived Suppressor Cell Infusion in Non-human Primate Kidney Transplantation,” Transplant Immunology 56:101225; Fitch, Z., Schmitz, R., Kwun, J., Hering. B., Madsen, J., and Knechtle, S. J. 2019. “Transplant Research in Nonhuman Primates to Evaluate Clinically Relevant Immune Strategies in Organ Transplantation,” Transplantation Reviews (Orlando, Florida) 33(3): 115-129; Meyer, E. H., Laport, G., Xie, B. J., MacDonald, K., Heydari, K., and 16 others 2019. “Transplantation of Donor Grafts with Defined Ratio of Conventional and Regulatory T Cells in HLA-matched Recipients,” Journal of Clinical Investigation Insight 4(10). pii: 127244; Nakamura, K., Kageyama, S., and Kupiec-Weglinski, J. W. 2019. “Innate Immunity in Ischemia-Reperfusion Injury and Graft Rejection,” Current Opinion in Organ Transplantation October 4; Sanada, T. J., Tanabe, N., Ishibashi-Ueda, H., Ishida, K., Naito, A., and 9 others 2019. “Involvement of Pulmonary Arteriopathy in the Development and Severity of Reperfusion Pulmonary Edema after Pulmonary Endarterectomy,” Pulmonary Circulation 9(2): 2045894019846439; Thangavelu, G. and Blazar, B. R. 2019; Op cit., in this section; Thomson, A. W., Metes, D. M., Ezzelarab, M. B., and Raich-Regue, D. 2019. Op cit., in this section; Wang, X., O'Brien, M. E., Yu, J., Xu. C., Zhang, Q., and 5 others 2019. “Prolonged Cold Ischemia Induces Necroptotic Cell Death in Ischemia-Reperfusion Injury and Contributes to Primary Graft Dysfunction after Lung Transplantation,” American Journal of Respiratory Cell and Molecular Biology 61(2): 244-256; Zhai, C., Lv. J., Wang, K., Li, Q., and Qu, Y. 2019. “HSP70 [heat shock protein 70] Silencing Aggravates Apoptosis Induced by Hypoxia/Reoxygenation in Vitro,” Experimental and Therapeutic Medicine 18(2): 1013-1020; Cheng, Q., Patel, K., Lei, B., Rucker, L., Allen, D. P., and 6 others 2018. “Donor Pretreatment with Nebulized Complement C3a Receptor Antagonist Mitigates Brain-death Induced Immunological Injury Post-Lung Transplant,” American Journal of Transplantation 18(10): 2417-2428; Kaundal, U., Bagai, U., and Rakha, A. 2018. “Immunomodulatory Plasticity of Mesenchymal Stem Cells: A Potential Key to Successful Solid Organ Transplantation,” Journal of Translational Medicine 16(1): 31: Kim, H., Zamel. R., Bai, X. H., Lu, C., Keshavjee, S., Keshavjee, S., and Liu, M. 2018. “Ischemia-Reperfusion Induces Death Receptor-independent Necroptosis via Calpain-STAT3 [signal transducer and activator of transcription 3] Activation in a Lung Transplant Setting,” American Journal of Physiology. Lung Cellular and Molecular Physiology 315(4): L595-L608; Pollara, J., Edwards, R. W., Lin, L., Bendersky, V. A., and Brennan, T. V. 2018. “Circulating Mitochondria in Deceased Organ Donors are Associated with Immune Activation and Early Allograft Dysfunction,” Journal of Clinical Investigation Insight 3(15). pii: 121622; Steinmeyer, J., Becker, S., Avsar, M., Salman, J., Hoffler, K., and 5 others 2018. “Cellular and Acellular ex Vivo Lung Perfusion Preserve Functional Lung Ultrastructure in a Large Animal Model: A Stereological Study,” Respiratory Research 19(1): 238; Zhou, H., Wang, S., Hu. S., Chen. Y., and Ren, J. 2018. “ER [endoplasmic reticulum]-Mitochondria Microdomains in Cardiac Ischemia-Reperfusion Injury: A Fresh Perspective,” Online, Frontiers in Physiology 9:755: Dai, S., Xu, Q., Liu, S., Yu, B., Liu, J., and Tang, J. 2017. “Role of Autophagy and Its Signaling Pathways in Ischemia/Reperfusion Injury,” American Journal of Translational Research 9(10): 4470-4480; Mahr. B., Granofszky, N., Muckenhuber, M., and Wekerle, T. 2017. “Transplantation Tolerance through Hematopoietic Chimerism: Progress and Challenges for Clinical Translation,” Online, Frontiers in Immunology (Lausanne, Switzerland) 8:1762; Makdisi, G., Makdisi. T., Jarmi. T., and Caldeira, C. C. 2017. “Ex Vivo Lung Perfusion Review of a Revolutionary Technology,” Annals of Translational Medicine 5(17): 343; Strober, S. 2016. “Use of Hematopoietic Cell Transplants to Achieve Tolerance in Patients with Solid Organ Transplants,” Blood 127(12): 1539-1543; Scandling, J. D., Busque, S., Shizuru, J. A., Lowsky, R., Hoppe. R., and 8 others 2015. “Chimerism, Graft Survival, and Withdrawal of Immunosuppressant Drugs in HLA Matched and Mismatched Patients after Living Donor Kidney and Hematopoietic Cell Transplantation,” American Journal of Transplantation 15(3): 695-704; Wallinder, A., Ricksten, S. E., Silverbom, M., Hansson, C., Riise, G. C., and 3 others 2014. “Early Results in Transplantation of Initially Rejected Donor Lungs after Ex Vivo Lung Perfusion: A Case-control Study,” European Journal of Cardiothoracic Surgery 45(1): 40-45; Fischer, S., Cassivi, S. D., Xavier, A. M., Cardella, J. A., Cutz, E., and 3 others 2000. “Cell Death in Human Lung Transplantation: Apoptosis Induction in Human Lungs during Ischemia and after Transplantation,” Annals of Surgery (Philadelphia, Pennsylvania) 231(3): 424-431; Fischer, S., Maclean, A. A., Liu, M., Cardella, J. A., Slutsky, A. S., and 3 others 2000. “Dynamic Changes in Apoptotic and Necrotic Cell Death Correlate with Severity of Ischemia-Reperfusion Injury in Lung Transplantation,” American Journal of Respiratory and Critical Care Medicine 162(5): 1932-1939; Stammberger, U., Gaspert, A., Hillinger, S., Vogt P., Odermatt. B., Weder, W., and Schmid, R. A. 2000. “Apoptosis Induced by Ischemia and Reperfusion in Experimental Lung Transplantation,” Annals Thoracic Surgery 69(5): 1532-1536; additional references cited in other sections, such as 2a (3) and 2b (1)(b).

Caustically, to the molecular and cellular level degradation products of death, reperfusion injury then adds further obstacles to the success of transplantation (see, for example. Dashkevich, A., Raissadati, A., Syrjäla, S. O., Zarkada, G., Keränen, M. A., and 8 others 2016. “Ischemia-Reperfusion Injury Enhances Lymphatic Endothelial VEGFR3 [vascular endothelial growth factor receptor 3] and Rejection in Cardiac Allografts,” American Journal of Transplantation 16(4): 1160-1172; Pei. G., Yao, Y., Yang, Q., Wang, M., Wang. Y., and 14 others 2019. “Lymphangiogenesis in Kidney and Lymph Node Mediates Renal Inflammation and Fibrosis,” Science Advances 5(6): eaaw5075; Heinolainen, K., Karaman, S., D'Amico, G., Tammela. T., Sormunen, R., and 3 others 2017. “VEGFR3 Modulates Vascular Permeability by Controlling VEGF/VEGFR2 Signaling,” Circulation Research 120(9): 1414-1425).

As currently practiced, the two major obstacles to successful transplantation are, first, immune tolerance aside, the need to optimize the condition of the graft organ, usually taken from a recently deceased patient, and second, the need to optimize the genetic compatibility between graft and recipient. While the ability to avoid rather than have to secondarily ameliorate this posthumous deterioration would fundamentally elevate the odds for successful outcomes, the standard practice remains harvesting the organ and then preserving it with or without remediation of both deteriorative and immunological obstacles. Provided the time since the terminal event will allow, new strategies currently under study, to include exogenous stem cell therapy, do appear to offer some degree of recoverability. Were these methods fully successful, the problem of optimizing organs that become available only following death-currently the rule-would be substantially reduced.

Emerging practices, notably stem cell therapy, readily integrated into compound vascular bypass, or sudden switched-organ transplantation along with immunosuppressants, do appear to have the potential both to reverse the cellular damage of death as well as initiate immune tolerance, so that the pursuit of both objectives proceeds simultaneously in parallel, and moreover, appears to protect against cold ischemia-reperfusion injury. Autologous stem cells aid healing, while allogeneic stem cells can induce immune tolerance as well as aid healing (see, for example, Hull, T. D., Benichou, G., and Madsen, J. C. 2019. “Why Some Organ Allografts are Tolerated Better than Others: New Insights for an Old Question,” Current Opinion in Organ Transplantation 24(1): 49-57; Kawai, T., Leventhal, J., Wood, K., and Strober, S. 2019. “Summary of the Third International Workshop on Clinical Tolerance,” American Journal of Transplantation 19(2): 324-330; Lee, K. H., Tseng, W. C., Yang, C. Y., and Tarng, D. C. 2019. “The Anti-inflammatory, Anti-oxidative, and Anti-apoptotic Benefits of Stem Cells in Acute Ischemic Kidney Injury,” Online, MDPI [Multidisciplinary Digital Publishing Institute] International Journal of Molecular Sciences (Basel, Switzerland) 20(14). pii: E3529; Podestà, M. A., Remuzzi, G., and Casiraghi, F. 2019. “Mesenchymal Stromal Cells for Transplant Tolerance,” Online, Frontiers in Immunology (Lausanne, Switzerland) 10:1287; Pool. M., Leuvenink, H., and Moers, C. 2019. “Reparative and Regenerative Effects of Mesenchymal Stromal Cells-Promising Potential for Kidney Transplantation?,” Online, MDPI [Multidisciplinary Digital Publishing Institute] International Journal of Molecular Sciences (Basel, Switzerland) 20(18). pii: E4614; Rota, C., Morigi, M., and Imberti, B. 2019. “Stem Cell Therapies in Kidney Diseases: Progress and Challenges,” Online, MDPI [Multidisciplinary Digital Publishing Institute] International Journal of Molecular Sciences (Basel, Switzerland) 20(11). pii: E2790; Casiraghi, F., Perico, N., and Remuzzi, G. 2018. “Mesenchymal Stromal Cells for Tolerance Induction in Organ Transplantation,” Human Immunology 79(5): 304-313; Hill, L., Alousi, A., Kebriaei, P. Mehta. R., Rezvani, K., and Shpall, E. 2018. “New and Emerging Therapies for Acute and Chronic Graft versus Host Disease,” Therapeutic Advances in Hematology 9(1): 21-46; Leventhal, J. R. and Ildstad, S. T. 2018. “Tolerance Induction in HLA [human leukocyte antigen] Disparate Living Donor Kidney Transplantation by Facilitating Cell-enriched Donor Stem Cell Infusion: The Importance of Durable Chimerism,” Human Immunology 79(5): 272-276; Sieńko, J., Kotowski, M., Paczkowska, E., Sobuś, A., Tejchman, K., and 4 others 2018, Op cit. above early in this section; Garakani, R. and Saidi. R. F. 2017. “Recent Progress in Cell Therapy in Solid Organ Transplantation,” International Journal of Organ Transplantation Medicine 20178(3): 125-131; Khoury, H. J., Wang, T., Hemmer, M. T., Couriel, D., Alousi. A., and 42 others 2017. “Improved Survival after Acute Graft-versus-host Disease Diagnosis in the Modern Era,” Online, Haematologica (Pavia, Italy) 102(5): 958-966; Casiraghi, F., Perico, N., Cortinovis, M., and Remuzzi, G. 2016. “Mesenchymal Stromal Cells in Renal Transplantation: Opportunities and Challenges,” Nature Reviews. Nephrology 12(4): 241-253; Jaime-Perez, J. C., Villarreal-Villarreal. C. D., Mendez-Ramirez, N., Vázquez-Garza, E., Salazar-Riojas, R., and Gomez-Almaguer, D. 2016. “Assessment of Immune Reconstitution Status in Recipients of a Successful Hematopoietic Stem Cell Transplant from Peripheral Blood after Reduced Intensity Conditioning,” Blood Cells, Molecules, and Diseases 58:52-56; Cortinovis, M., Casiraghi, F., Remuzzi, G., and Perico, N. 2015. “Mesenchymal Stromal Cells to Control Donor-specific Memory T cells in Solid Organ Transplantation,” Current Opinion in Organ Transplantation 20(1): 79-85; Strober, S. 2016. Op cit., this section; Bank, J. R., Rabelink, T. J., de Fijter, J. W., and Reinders, M. E. 2015. “Safety and Efficacy Endpoints for Mesenchymal Stromal Cell Therapy in Renal Transplant Recipients,” Journal of Immunology Research 2015:391797; Scandling, J. D., Busque, S., Shizuru, J. A., Lowsky, R., Hoppe, R., and 8 others 2015, Op cit., this section; Tian, W., Liu, Y., Zhang, B., Dai, X., Li, G., and 4 others 2015. “Infusion of Mesenchymal Stem Cells Protects Lung Transplants from Cold Ischemia-Reperfusion Injury in Mice,” Lung 193(1): 85-95; Lee, H., Park, J. B., Lee, S., Baek, S., Kim, H., and Kim, S. J. 2013. “Intra-osseous Injection of Donor Mesenchymal Stem Cell (MSC) into the Bone Marrow in Living Donor Kidney Transplantation; a Pilot Study,” Journal of Translational Medicine 11:96; Leventhal, J., Abecassis, M., Miller, J., Gallon, L., Tollerud, D., and 5 others 2013. “Tolerance Induction in HLA Disparate Living Donor Kidney Transplantation by Donor Stem Cell Infusion: Durable Chimerism Predicts Outcome,” Transplantation 95(1): 169-176; Peng. Y., Ke. M., Xu. L., Liu, L., Chen, X., and 9 others 2013. “Donor-derived Mesenchymal Stem Cells Combined with Low-dose Tacrolimus Prevent Acute Rejection after Renal Transplantation: A Clinical Pilot Study,” Transplantation 95(1): 161-168; Perico, N., Casiraghi, F., Gotti, E., Introna, M., Todeschini, M., and 8 others 2013. “Mesenchymal Stromal Cells and Kidney Transplantation: Pretransplant Infusion Protects from Graft Dysfunction while Fostering Immunoregulation,” Transplant International 26(9): 867-878; Reinders, M. E., de Fijter, J. W., Roelofs, H., Bajemna, I. M., de Vries, D. K., and 7 others 2013. “Autologous Bone Marrow-derived Mesenchymal Stromal Cells for the Treatment of Allograft Rejection after Renal Transplantation: Results of a Phase 1 Study,” Stem Cells Translational Medicine 2(2): 107-111; Leventhal, J., Abecassis, M., Miller, J., Gallon, L., Ravindra, K., and 6 others 2012. “Chimerism and Tolerance without GVHD [graft versus host disease] or Engraftment Syndrome in HLA [human leucocyte antigen]-mismatched Combined Kidney and Hematopoietic Stem Cell Transplantation,” Science Translational Medicine 4(124): 124-128. Online at https://www.nebi.nlm.nih.gov/pme/articles/PMC 3610325/pdf/nihms-452051. pdf; Tan, J., Wu. W., Xu X., Liao. L., Zheng. F., and 8 others 2012. “Induction Therapy with Autologous Mesenchymal Stem Cells in Living-related Kidney Transplants: A Randomized Controlled Trial,” Journal of the American Medical Association 307(11): 1169-1177; Vanikar, A. V. and Trivedi, H. L. 2012. “Stem Cell Transplantation in Living Donor Renal Transplantation for Minimization of Immunosuppression,” Transplantation 94(8): 845-850; additional references cited in section 2b(1)(b) and its subordinate sections, for example.

However, when the patient dies having consented, ex vivo preservation should be replaced with in vivo sustainment. Death and preservation severely degrading the viability and transplantability of graft organs, when a patient is terminal in the center, the body can be sustained in a nearly optimal condition for the organs to be transplanted when the patient finally dies and for an interval beyond (see, for example, (Shetty, V. L., Mali, S. S., Shetty, S. V., and Shinde, P. D. 2017. Op cit. above in section 2a). Once the patient enters into a state of irreversible coma with death imminent, the process of preparing for the graft organs to be harvested is initiated. The prospective recipient is positioned alongside the donor and placement of the valves or servovalves for organ transfer initiated.

Where the law prevents organ harvesting despite the depth of coma prior to death, placement of the automatic disorder response system and life support initiated prior to death and not turned off upon death or until organ harvesting has been completed should prove adequate. That the switch method continuously sustains perfusion and oxygenation throughout the transfer of the graft organ from the donor to the recipient—with a gradual transition to the blood of the recipient in a metered switch—is of central benefit in the avoidance of ischemia-reperfusion injury and most likely post-transplantation vasculopathy, which is the cause for the failure in large percentage of heart transplants.

Furthermore, the switch methods leave intact and therefore impose much less trauma on graft organs than do conventional procedures. The objective sought are to isolate graft organs from the chemical consequences of a cessation in perfusion and oxygenation associated with death, and transfer the organs into recipients using a continuous blood exchange process that gradually transitions, or acclimates, the organ from the circulatory system of the donor into that of the recipient. Simply stated, an effort is made to conceal from graft organs the death of the host and transfer into the circulatory system of another. This is enhanced in metered compound vascular bypass, or sudden switched-organ transplantation, which seeks to accomplish immune tolerance induction as an integral component of the continuous blood transfer.

By suddenly transferring the donor, or graft, organ from the circulatory system of the donor into that of the recipient without interruption in perfusion, and if a lung, without interruption in spontaneous or natural breathing, this technique reduces the causes for graft failure other than immune intolerance. Metered compound vascular bypass, or sudden switched-organ transplantation affects the transfer of the graft organ between the circulatory systems gradually, so that the interval over which recipient and donor are exposed to one another's blood is extended. Perfusion of the graft organ with the blood of the recipient gradually increases in relative proportion until the organ is transferred to the recipient.

This process of reciprocal cross-circulation where the blood of the recipient and donor gradually blend provides a measure of immune tolerance induction, thus addressing the remaining obstacle to success in transplantation. After transplantation, the implanted automatic disorder response system will continue the administration of immunosuppressants, but in a directly targeted manner that does not immunocompromise the patient. Minimization in the ability of the graft organ to recognize its alien new milieu and of the recipient to recognize the fact of an alien intrusion allows transplantation which if not completely adverse reaction-free, is at least as unprovocative and uneventful as might reasonably be accomplished.

While delivered to the donor organ as a constituent in the blood of the recipient-which does necessitate larger dosing to compensate for the dilution factor—the recipient-donor reciprocal cross-chimerization achieved by reciprocal cross-circulation in metered compound vascular bypass, or sudden switched-organ transplantation inherently targets the gene transfer vector directly to the graft organ of the recipient, augmenting the chimerization provided by reciprocal cross-circulation alone. The removal of plasma or serum from the blood of the donor, which has been found to interfere with transgene uptake is easily accomplished along the mainlines, or bloodlines, joining donor and recipient (Bishawi, M., Roan, J. N., Milano, C. A., Daneshmand, M. A., Schroder, J. N., and 10 others 2019. Op cit., this section).

Delivery essentially tracks Bishawi et al. but is adapted to the in vivo arrangement used in metered compound vascular bypass, or sudden switched-organ transplantation rather than the ex vivo arrangement imposed by use of the Organ Care System™ (TransMedics Incorporated, Andover, Massachusetts), for example. Where ex vivo heart and lung perfusion machines seek to minimize ischemia, switch transplantation avoids ischemia entirely, but does require that the bodily systems of the donor be sustained past death and the recipient be positioned alongside the donor. The donor and recipient are positioned to afford the smallest distance between the greatest number of vessels to be connected, the organs involved aligned in adjacent relation.

Except when to connect all the vessels would not necessitate eventually having to rotate either the donor or recipient, the relatively small differences in distance between like vessels of paired and unpaired organs due to chirality with donor and recipient uninverted, with both heads up or inverted, with one head up and the other down are discounted. Unless significant differences in organ to organ distance should be disregarded. Where ex vivo perfusion machines encourage the donor heart to be removed for pretransplantation gene therapy, metered compound vascular bypass, or sudden switched-organ transplantation effects gene therapy as integrated into the reciprocal cross-circulation and reciprocal preliminary cross-chimerization between donor and recipient. Ex vivo graft hearts may be maintained in an anoxic state for hours prior to machine perfusion, all the while accumulating constituents in the blood inimical to a successful outcome.

Metered compound vascular bypass, or switched-organ transplantation can progress for a period considerably longer than the two hours used by Bishawi, et al. (see, also Ishikawa, K., Weber, T., and Hajjar, R. J. 2018. “Human Cardiac Gene Therapy,” Circulation Research 123(5): 601-613; Jean-Charles, P. Y., Yu, S. M., Abraham, D., Kommaddi, R. P., Mao. L., and 9 others 2017. “Mdm2 Regulates Cardiac Contractility by Inhibiting GRK2-mediated Desensitization of β-adrenergic Receptor Signaling,” Journal of Clinical Investigation Insight 2(17). pii: 95998; Matkar. P. N., Leong-Poi, H., and Singh, K. K. 2016. “Cardiac Gene Therapy: Are We There Yet?,” Gene Therapy 23(8-9): 635-648; Miyagi, N., Rao, V. P., Ricci, D., Du, Z., Byrne, G. W., and 4 others 2008. “Efficient and Durable Gene Transfer to Transplanted Heart Using Adeno-associated Virus 9 Vector,” Journal of Heart and Lung Transplantation (New York, New York) 27(5): 554-560).

As well as to supplement the chimerizing effect of reciprocal cross-circulation, gene therapy incorporated into metered compound vascular bypass, or sudden switched-organ transplantation can also be used, alone or with stem cells and drugs, to restore a subcritically failing donor heart, lung, or other solid organ, thus further increasing the pool of organs available for transplant, and while ex vivo perfusion machines also allows this, the numerous detractions attendant upon such use, of which none pertain to metered compound vascular bypass, or sudden switched-organ transplantation, make the latter preferable for such treatment (see, for example, Bulaklak, K., and Gersbach, C. A. 2020. Op cit., above in section 2a, entitled Background. General).

The term “heart failure” without qualification usually refers to heart failure with reduced ejection fraction (absolute stroke volume)(see, for example, Bishawi, M., Roan, J. N., Milano, C. A., Daneshmand, M. A., Schroder, J. N., and 10 others 2019, Op cit.; Choi, H. M., Park, M. S., and Youn, J. C. 2019. “Update on Heart Failure Management and Future Directions,” Korean Journal of Internal Medicine 34(1): 11-43; Guenther, C. M., Brun, M. J., Bennett, A. D., Ho, M. L., Chen, W., and 11 others 2019. “Protease-activatable Adeno-associated Virus Vector for Gene Delivery to Damaged Heart Tissue,” Molecular Therapy 27(3): 611-622; Kieserman, J. M., Myers, V. D., Dubey, P., Cheung, J. Y., and Feldman, A. M. 2019. “Current Landscape of Heart Failure Gene Therapy,” Journal of the American Heart Association (Oxford, England) 8(10): e012239; Patel, R. B., Nohria, A., Butler, J., and Vaduganathan, M. 2019. “Dying is Not What It Used to Be! Impact of Evolving Epidemiology and Treatment on Mode of Death in Heart Failure,” European Journal of Heart Failure 21(10): 1267-1269; Sendra, L., Herrero, M. J., Montalva, E. M., Noguera, I., Orbis. F., and 4 others 2019. “Efficacy of Interleukin 10 Gene Hydrofection in Pig Liver Vascular Isolated ‘in Vivo’ by Surgical Procedure with Interest in Liver Transplantation,” Online, PLoS [Public Library of Science] One 14(11): e0224568; Gabisonia, K. and Recchia, F. A. 2018. “Gene Therapy for Heart Failure: New Perspectives,” Current Heart Failure Reports 15(6): 340-349; Katz, M. G., Fargnoli, A. S., Yamall. C., Perez, A., Isidro, A., Hajjar, R. J., and Bridges, C. R. 2018. “Technique of Complete Heart Isolation with Continuous Cardiac Perfusion during Cardiopulmonary Bypass: New Opportunities for Gene Therapy,” Journal of Extracorporeal Technology 50(3): 193-198; Penny, W. F., Henry, T. D., Watkins, M. W., Patel, A. N., and Hammond, H. K. 2018. “Design of a Phase 3 Trial of Intracoronary Administration of Human Adenovirus 5 Encoding Human Adenylyl Cyclase Type 6 (RT-100) Gene Transfer in Patients with Heart Failure with Reduced Left Ventricular Ejection Fraction: The FLOURISH [not an acronym] Clinical Trial,” American Heart Journal 201:111-116; Sendra, L., Herrero, M. J., and Aliño, S. F. 2018. “Translational Advances of Hydrofection by Hydrodynamic Injection,” Genes (Basel, Switzerland) 9(3). pii: E136; Vaduganathan, M., Patel, R. B., Mentz, R. J., Subacius, H., Chatterjee, N. A., and 10 others 2018. “Sudden Death after Hospitalization for Heart Failure with Reduced Ejection Fraction (from the EVEREST [Efficacy of Vasopressin Antagonism in Heart Failure Outcome Study with Tolvaptan] Trial),” American Journal of Cardiology 122(2): 255-260; Yin, Z. Q. and Xing. W. H. 2018. “Progress in Gene Therapy for Chronic Heart Failure,” Heart Surgery Forum 21(2): E075- E083; Katz, M. G., Fargnoli, A. S., Hajjar, R. J., and Bridges, C. R. 2017. “In Situ Heart Isolation Featuring Closed Loop Recirculation: The Gold Standard for Optimum Cardiac Gene Transfer?,” Advanced Techniques in Biology Medicine 5(4). pii: 241; Katz, M. G., Fargnoli, A. S., Kendle, A. P., and Bridges, C. R. 2017. “Molecular Cardiac Surgery with Recirculating Delivery (MCARD): Procedure and Vector Transfer,” Methods in Molecular Biology 1521:271-289; Metra, M. and Teerlink, J. R. 2017. “Heart Failure,” Lancet 390(10106): 1981-1995; Penny, W. F. and Hammond, H. K. 2017. “Randomized Clinical Trials of Gene Transfer for Heart Failure with Reduced Ejection Fraction,” Human Gene Therapy 28(5): 378-384; Yla-Herttuala. S. and Baker, A. H. 2017. “Cardiovascular Gene Therapy: Past, Present, and Future,” Molecular Therapy 25(5): 1095-1106; Greenberg, B., Butler, J., Felker, G. M., Ponikowski, P., Voors. A. A., and 8 others 2016. “Calcium Upregulation by Percutaneous Administration of Gene Therapy in Patients with Cardiac Disease (CUPID 2): A Randomised, Multinational, Double-blind, Placebo-controlled, Phase 2b Trial,” Lancet 387(10024): 1178-1186; Sendra. L., Miguel. A., Perez-Enguix, D., Herrero, M. J., Montalva, E., and 8 others 2016. “Studying Closed Hydrodynamic Models of “In Vivo” DNA Perfusion in Pig Liver for Gene Therapy Translation to Humans,” Online, PLoS [Public Library of Science] One 11(10): e0163898; Tilemann, L., Ishikawa, K., Weber. T., and Hajjar, R. J. 2012. “Gene Therapy for Heart Failure,” Circulation Research 110(5): 777-793).

Outside of transplantation, in vivo organ isolation and perfusion as in isolated hepatic perfusion is likewise superior to systemic treatment. The use of ductus side-entry diversion chute servovalves and nonjacketing side-entry connectors in a totally implanted system as described in U.S. Pat. No. 11,759,186, entitled Ductus Side-entry Jackets and Prosthetic Disorder Response Systems would however, provide more dependable, balloon catheter complication and leak-free, and flow-controllable connections for the catheters, and the targeting of supplemental or adjuvant agents to the same organ through the jacket accessory channels.

The use of these components would allow isolated hepatic perfusion, conventionally used to target stem cell and chemotherapy to the liver but readily adaptable to the targeting of restorative, and in transplantation, chimerizing gene therapy, as well as the targeted administration of tumor necrosis factor, to continue over an indefinite period (see, for example, Ramadan, K., Del Sorbo, L., and Cypel, M. 2019. “In Vivo Lung Perfusion as a Platform for Organ Repair in Acute Respiratory Distress Syndrome,” Journal of Thoracic Disease 11(1): 30-34; Boone, B. A., Perkins, S., Bandi, R., Santos, E., Mccluskey, K., Bartlett, D. L., and Pingpank, J. F. 2018. “Hepatic Artery Infusion of Melphalan in Patients with Liver Metastases from Ocular Melanoma,” Journal of Surgical Oncology 117(5): 940-946; Mehaffey, J. H., Charles, E. J., Schubert, S., Salmon, M., Sharma, A. K., and 6 others 2018. “In Vivo Lung Perfusion Rehabilitates Sepsis-induced Lung Injury,” Journal of Thoracic and Cardiovascular Surgery 155(1): 440-448.e2; von Hom, C. and Minor, T. 2018. “Isolated Kidney [ex vivo in transplantation] Perfusion: The Influence of Pulsatile Flow,” Scandinavian Journal of Clinical and Laboratory Investigation 78(1-2): 131-135; Cypel, M. and Keshavjee, S. 2016. “Novel Technologies for Isolated Lung Perfusion: Beyond Lung Transplant,” Thoracic Surgery Clinics 26(2): 139-145; Reck Dos Santos, P., Sakamoto, J., Chen, M., Linacre, V., Arce, C., and 4 others 2016. “Modified in Vivo Lung Perfusion for Local Chemotherapy: A Preclinical Study with Doxorubicin,” Annals of Thoracic Surgery 101(6): 2132-2140; dos Santos, P. R., Iskender, I., Machuca, T., Hwang. D., dePerrot, M., and 4 others 2014. “Modified in Vivo Lung Perfusion Allows for Prolonged Perfusion without Acute Lung Injury,” Journal of Thoracic and Cardiovascular Surgery 147(2): 774-782; Raper, S. E., Wilson, J. M., and Nunes, F. A. 2013. “Flushing Out Antibodies to Make AAV [adeno-associated virus] Gene Therapy Available to More Patients,” Molecular Therapy 21(2): 269-227; Cypel, M. and Keshavjee, S. 2012. “Isolated Lung Perfusion,” Online, Frontiers in Bioscience (Elite Edition) 4:2226-2232; O Donnell, J. M., Kalichira, A., Bi. J., and Lewandowski, E. D. 2012. “In Vivo, Cardiac-specific Knockdown of a Target Protein, Malic Enzyme-1, in Rat via Adenoviral Delivery of DNA for Non-native miRNA [microRNA],” Current Gene Therapy 12(6): 454-462; Varghese, S., Xu. H., Bartlett, D., Hughes, M., Pingpank, J. F., Beresnev. T., and Alexander, H. R. Jr. 2010. “Isolated Hepatic Perfusion with High-dose Melphalan Results in Immediate Alterations in Tumor Gene Expression in Patients with Metastatic Ocular Melanoma,” Annals of Surgical Oncology 17(7): 1870-1877; Wang, X. D. and Yang, R. J. 2008. “Isolated Hepatic Perfusion for Treatment of Unresectable Malignant Tumor,” (in Chinese with abstract at Pubmed) Beijing Da Xue Xue Bao Yi Xue Ban (Journal of Beijing (Peking) University. Health Sciences) 40(2): 225-228; van Etten, B., Eggermont, A. M., van Tiel, S. T., Ambagtsheer. G., de Wilt. J. H., and ten Hagen, T. L. 2005. “Gene Therapy in in Vivo Isolated Perfusion Models,” Current Gene Therapy 5(2): 195-202; Youngman, R. C., Klugo. R. C., Cruickshank, R. D., and Cemy, J. C. 1976. “A Technique for Isolated in Vivo Renal Perfusion,” Investigative Urology 14(3): 187-190).

Thus, taking these various factors into consideration, application to metered compound vascular bypass, or sudden switched-organ transplantation involves a fundamental improvement in changing the process from ex- to in vivo, of which the advantages include the elimination of pretransplantation organ harvesting and placement, eliminating ischemia and reperfusion injury, along with the cardioplegia, general anesthesia, extensive incision into both donor and recipient hearts, and so on, all potentially injurious and central in conventional heart transplantation. This will prove advantageous in the ability to not only augment the reciprocal cross-chimerization of the donor and recipient to facilitate solid organ acceptance but allow the transplantation preliminary and/or concurrent application of gene therapy as well as chimerization to increase the pool of available hearts by allowing the use of those subcritically failing for double heart transplantation, primarily in the elderly.

The process of gene transfer as incorporated into and concurrent with donor-recipient reciprocal cross-chimerization and physical transfer of the graft organ during reciprocal cross-circulation with the added advantage of gene therapy is no less applicable to the transplantation using the switch method of other solid organs. Also integrable is the introduction of agents such as thymosin β4, or timbetasin, which can be added to increase the plasma concentration of the donor-recipient shared bloodstream during switch transplantation to reduce inflammation and facilitate graft survival (see, for example, Dube, K. N. and Smart, N. 2018. “Thymosin β4 and the Vasculature: Multiple Roles in Development, Repair, and Protection against Disease,” Expert Opinion on Biological Therapy 18 (Supplement 1—Proceedings of the Fifth International Symposium on Thymosins in Health and Disease): 131-139; Gupta, S. and Li, L. 2018. “The Role of Thymosin β4 in Angiotensin II-induced Cardiomyocytes Growth,” Expert Opinion on Biological Therapy 18 (Supplement 1—Proceedings of the Fifth International Symposium on Thymosins in Health and Disease): 105-110; Hinkel. R., Klett, K., Bähr, A., and Kupatt, C. 2018. “Thymosin β4-mediated Protective Effects in the Heart,” Expert Opinion on Biological Therapy 18 (Supplement 1—Proceedings of the Fifth International Symposium on Thymosins in Health and Disease): 121-129; Kumar, N., Liao, T. D., Romero, C. A., Maheshwari, M., Peterson, E. L., and Carretero, O. A. 2018. “Thymosin β4 Deficiency Exacerbates Renal and Cardiac Injury in Angiotensin-II-induced Hypertension,” Hypertension (Dallas, Texas) 71(6): 1133-1142; Postrach, J., Schmidt, M., Thormann, M., Thein, E., Burdorf. L., and 8 others 2014. “Adeno-associated Viral Vector 2.9 Thymosin β4 Application Attenuates Rejection after Heart Transplantation: Results of a Preclinical Study in the Pig,” Transplantation 98(8): 835-843). Accordingly, the subject is addressed below in sections 2b (1)(b), entitled Metered Compound Vascular Bypass, or Metered Switched-organ Transplantation, 2b (1)(b)(b4), entitled Metered Compound Vascular Bypass, or Switched-organ Transplantation in an Adult, and 2b (1)(b)(b5), entitled Heterotopic Double Heart Metered Compound Vascular Bypass, or Switched-organ Transplantation [and so on].

Furtherance in the dependability and efficacy as well as a reduction in the complexity of organ transplantation can be expected to encourage the abandonment of less effective, or ‘half-way,’ medical procedures. For example, perivascular stent-jackets and stay based stents as described in U.S. Pat. No. 11,389,171, entitled Integrated System for the Infixion and Retrieval of Implants, require placement through a small incision, making this an invasive procedure, while the transluminal placement of conventional stents is minimally invasive; however, the invasive factor is more than compensated for by certain distinct advantages. For another example pertinent to organ transplantation, transluminal repair of the severely malformed heart in utero, open surgical repair in the neonate or infant, and conventional replacement of the severely malformed heart usually save the life of the baby, but unless normal circulation ensues, results of the procedure over the long-term prove disappointing.

By contrast, orthotopic heart transplantation whether conventional or by means of the switch technique described herein can provide pulsatile rather than nonpulsatile or passive Fontan pulmonary arterial flow with sufficient oxygenation, thereby allowing normal development. Absent pulmonary vascular resistance, some transient left ventricular weakness should not occur. If it does, then the weakness should respond to the direct targeting to the ventricular myocardia of one or more positive inotropic agents, such as dopexamine, angiotensin II, a prostaglandin, or enoximone, alone or in a special formulation, over a duration sufficient to allow it to strengthen.

If positive inotropes would aggravate the underlying disease process, then vasoactive and neurohumoral agents are directly pipeline-targeted through a basic ductus side-entry jacket placed about the left anterior descending coronary artery in the manner described in U.S. Pat. No. 11,759,186, entitled Ductus Side-entry Jackets and Prosthetic Disorder Response Systems. The term ‘ejection fraction’ rather than ‘absolute stroke volume’ has been criticized as unindicative of the type of myopathy (Konstam, M. A. and Abboud, F. M. 2017. “Ejection Fraction: Misunderstood and Overrated (Changing the Paradigm in Categorizing Heart Failure)” Circulation 135(8): 717-719; Cikes, M and Solomon, S. D. 2016. “Beyond Ejection Fraction: An Integrative Approach for Assessment of Cardiac Structure and Function in Heart Failure,” European Heart Journal 37(21): 1642-1650; (see also, for example, Monge Garcia, M. I., Cecconi, M., and Pinsky, M. R. 2019. “Assessing Left Ventricular Systolic Function with Ejection Fraction: Using a Double-edged Knife as a Hammer,” Annals of Intensive Care 9(1): 111; Monge Garcia, M. I., Jian, Z., Settels, J. J., Hunley, C., Cecconi, M., Hatib. F., and Pinsky, M. R. 2019. “Determinants of Left Ventricular Ejection Fraction and a Novel Method to Improve Its Assessment of Myocardial Contractility,” Annals of Intensive Care 9(1): 48; Monge Garcia, M. I., Jian, Z., Settels, J. J., Hunley, C., Cecconi, M., Hatib, F., and Pinsky, M. R. 2018. “Performance Comparison of Ventricular and Arterial dP/dtmax [peak first derivative of femoral artery pressure] for Assessing Left Ventricular Systolic Function during Different Experimental Loading and Contractile Conditions,” Online, BioMed Central Critical Care (London, England) 22(1): 325: Ostadal, P., Vondrakova, D., Krüger, A., Janotka, M., and Naar, J. 2019. “Continual Measurement of Arterial dP/dtmax Enables Minimally Invasive Monitoring of Left Ventricular Contractility in Patients with Acute Heart Failure,” Critical Care 23(1): 364; Vaquer, S., Chemla, D., and Monnet, X. 2019. “When One Cannot See the Forest for the Trees: Femoral dP/dtmax, LVEF and Pulse Pressure in Critically Ill Patients,” Annals of Intensive Care 9(1): 112; Vaquer, S., Chemla, D., Teboul, J. L., Ahmad, U., Cipriani, F., and 5 others 2019. “Influence of Changes in Ventricular Systolic Function and Loading Conditions on Pulse Contour Analysis-derived Femoral dP/dtmax,” Annals of Intensive Care 9(1): 61; Pellikka, P. A., She, L., Holly, T. A., Lin, G., Varadarajan, P., and 15 others 2018. “Variability in Ejection Fraction Measured By Echocardiography, Gated Single-Photon Emission Computed Tomography, and Cardiac Magnetic Resonance in Patients with Coronary Artery Disease and Left Ventricular Dysfunction,” Journal of the American Medical Association Network Open 3;1(4): e181456; Kramer, D. G., Trikalinos, T. A., Kent, D. M., Antonopoulos, G. V., Konstam, M. A., and Udelson, J. E. 2010. “Quantitative Evaluation of Drug or Device Effects on Ventricular Remodeling as Predictors of Therapeutic Effects on Mortality in Patients with Heart Failure and Reduced Ejection Fraction: A Meta-analytic Approach,” Journal of the American College of Cardiology 56(5): 392-406).

Accordingly, a new dependability, facility, and relative simplicity should result in an increase in the number of individuals amenable to receiving a transplant and the number of medical professionals able to perform such operations. It follows that the availability of transplantation would reasonably be distributed from the largest centers to community hospitals. Here solid organ transplantation with zero ischemia time is implemented by means of a compound vascular bypass of the native organ through intravascular diversion servovalves and bloodlines the same in caliber as the vessels to which each is attached. The bypass transfers the organ from the circulatory system of the donor to that of the recipient. When the condition of the patient is urgent, the switch from recipient to donor heart is immediate, supporting therapy then accomplished following transplantation. When carried out gradually as described below under 1b(1)(b) entitled Metered Compound vascular bypass, or sudden switched-organ Transplantation, the procedure affords a measure of immune tolerance induction which also allows gene and stem cell therapy to initiate the restoration of a less than ideal replacement heart. Implanted suddenly, a replacement heart for a sudden compound vascular bypass, or sudden switched-organ transplantation must be less impaired than one meter compound vascular bypass, or sudden switched-organ implanted, where time can be taken to treat nondisqualifying deficits.

In sudden compound vascular bypass, or sudden switched-organ orthotopic heart transplantation, solenoid operated diversion valves are switched simultaneously from full flow through the native to full flow through the graft organ by actuation from the same plunger switch by the operator or an assistant. In metered compound vascular bypass, or sudden switched-organ orthotopic of heterotopic heart transplantation, this transfer of flow is also complete but accomplished gradually by small diversion servovalves driven by tiny linear servomotors.

Until adequate remedial measures have been developed, hearts for switched-organ transplantation with marginal heart failure will usually be of the ejection fraction reduced, or nonpreserved kind. The microprocessor executes a prescription-program that is written to respond to the various contingencies that might arise during the operation. Addressed below in section 2b(1)(b)(b5), entitled Heterotopic Double Heart Metered Compound Vascular Bypass, or Switched-organ Transplantation [and so on], is always metered, and the end-point requiring flow through either heart in proportion to its ejection fraction, the valves are stopped at about the halfway point.

As in conventional orthotopic heart transplantation, the replacement heart will usually be left partially denervated; however, where pain sensation is desired, the positioning of a ductus side-entry jacket or diversion servovalve at or about the anastomosis between severed ends of the vagus will allow the direct delivery to the site of immunosuppressant and other medication, as well as stem cells, to facilitate healing, or coaptation and fusion, between donor and recipient stumps.

In almost half of cases, the process of reinnervation can be confirmed as having begun by a year post-transplantation. “In conclusion, partial sympathetic reinnervation increases with time after HT [heart transplantation]; it was seen in 40% of patients at 1 year after the operation,” (Buendia-Fuentes, F., Almenar, L., Ruiz, C., Vercher, J. L., Sanchez-Lazaro, I., and 4 others 2011. “Sympathetic Reinnervation 1 Year after Heart Transplantation, Assessed Using Iodine-123 Metaiodobenzylguanidine Imaging,” Transplantation Proceedings 43(6): 2247-2248)(but see also Lee, S. R., Kang, D. Y., Cho, Y., Cho, H. J., Lee, H. Y., Choi, E. K., and Oh, S. 2016. “Early Parasympathetic Reinnervation is Not Related to Reconnection of Major Branches of the Vagus Nerve after Heart Transplantation,” Korean Circulation Journal 46(2): 197-206).

The vagus and superior cervical and inferior or thoracic cardiac branches of the vagus nerve, technically cranial, have been observed to eventually fuse following heart transplantation (see, for example, Beitzke, D., Wielandner, A., Wollenweber, T., Vraka, C., Pichler, V., and 5 others 2019. “Assessment of Sympathetic Reinnervation after Cardiac Transplantation Using Hybrid Cardiac PET/MRI: A Pilot Study,” Journal of Magnetic Resonance Imaging 50(4): 1326-1335; Dolan, R. S., Rahsepar, A. A., Blaisdell, J., Lin, K., Suwa, K., and 10 others 2019. “Cardiac Structure-Function MRI in Patients after Heart Transplantation,” Journal of Magnetic Resonance Imaging 49(3): 678-687; Moura-Tonello, S. C. G. de, Carvalho, V. O., Godoy, M. F., Porta, A., Leal, A. M. O., Bocchi, E. A., and Catai, A. M. 2019. “Evaluation of Cardiac Autonomic Modulation Using Symbolic Dynamics after Cardiac Transplantation,” Brazilian Journal of Cardiovascular Surgery 34(5): 572-580; Sinha, T., Lyon, A., and Kahwash, R. 2019. “A Case Report: Pause and Consider the Late Complications of Heart Transplantation,” European Heart Journal. Case Reports 3(3): ytz107; Grupper, A., Gewirtz, H., and Kushwaha, S. 2018. “Reinnervation Post-heart Transplantation,” European Heart Journal 39(20): 1799-1806; Wdowczyk, J., Makowiec, D., Gruchala, M., Wejer, D., and Struzik, Z. R. 2018. “Dynamical Landscape of Heart Rhythm in Long-term Heart Transplant Recipients: A Way to Discern Erratic Rhythms,” Online, Frontiers in Physiology 9:274, Peçanha, T., Bartels, R., Brito, L. C., Paula-Ribeiro, M., Oliveira, R. S., and Goldberger, J. J. 2017. “Methods of Assessment of the Post-exercise Cardiac Autonomic Recovery: A Methodological Review,” International Journal of Cardiology 227:795-802; Takakura, I. T., Hoshi, R. A., Santos, M. A., Pivatelli, F. C., Nobrega, J. H., and 5 others 2017. “Recurrence Plots: A New Tool for Quantification of Cardiac Autonomic Nervous System Recovery after Transplant,” Brazilian Journal of Cardiovascular Surgery 32(4): 245-252; Awad, M., Czer, L. S., Hou, M., Golshani, S. S., Goltche, M., and 8 others 2016. “Early Denervation and Later Reinnervation of the Heart following Cardiac Transplantation: A Review,” Journal of the American Heart Association (Oxford, England) 5(11). pii: e004070; Bravo, P. E., Lautamaki, R., Carter, D., Holt, D. P., Nekolla, S. G., and 3 others 2015. “Mechanistic Insights into Sympathetic Neuronal Regeneration: Multitracer Molecular Imaging of Catecholamine Handling after Cardiac Transplantation,” Circulation. Cardiovascular Imaging 8(8): e003507; Buendia-Fuentes, F., Almenar, L., Ruiz, C., Vercher, J. L., Sanchez-Lazaro, I., and 4 others 2011, Op cit., preceding paragraph; Andreassen, A. K. 2008. “Point: Counterpoint: Cardiac Denervation Does/Does Not Play a Major Role in Exercise Limitation after Heart Transplantation,” Journal of Applied Physiology 104(2): 559-564; Murphy, D. A., Thompson, G. W., Ardell, J. L., McCraty, R., Stevenson, R. S., and 7 others 2000. “The Heart Reinnervates after Transplantation,” Annals of Thoracic Surgery 69(6): 1769-1781; Uberfuhr, P., Frey, A. W., and Reichart, B. 2000. “Vagal Reinnervation in the Long-term after Orthotopic Heart Transplantation,” Journal of Heart and Lung Transplantation (New York, New York) 19(10): 946-950; Tio, R. A., Reyners, A. K., van Veldhuisen, D. J., van den Berg, M. P., Brouwer, R. M., and 3 others 1997. “Evidence for Differential Sympathetic and Parasympathetic Reinnervation after Heart Transplantation in Humans,” Journal of the Autonomic Nervous System 67(3): 176-183).

The modulation of the heartbeat, much less its communication with the rest of the body through the bloodstream, however, is not exclusively confined to the parasympathetic fibers of the vagus and the sympathetic fibers of the accelerans nerve. Nevertheless, by definition, the reinstatement of normal sensation that would allow the patient to become aware of a cardiac or coronary malfunction is to be preferred.

Adjustment of cardiac function responsive to orthostatic hypotension upon rising from a reclined to an upright position is less serious but can result in syncope, or the loss of consciousness (see, for example, Nygaard, S., Christensen, A. H., Rolid, K., Nytroen, K., Gullestad, L., and 6 others 2019. “Autonomic Cardiovascular Control Changes in Recent Heart Transplant Recipients Lead to Physiological Limitations in Response to Orthostatic Challenge and Isometric Exercise,” European Journal of Applied Physiology 119(10): 2225-2236; Low. P. A. and Tomalia, V. A. 2015. “Orthostatic Hypotension: Mechanisms, Causes, Management,” Journal of Clinical Neurology 11(3): 220-226; Hunt, S. 2001. “Reinnervation of the Transplanted Heart-Why is it Important?,” New England Journal of Medicine 345(10): 762-764).

For now, coaptation and fusion of peripheral nerves is possible, although with the exception of the long-term fusion of the vagus following heart transplantation, central nervous tissue, in particular, the optic cranial nerves to allow whole-eye transplantation, remains elusive (Canavero, S. and Ren, X. 2019. “Advancing the Technology for Head Transplants: From Immunology to Peripheral Nerve Fusion,” Surgical Neurology International 10:240). For a double heart transplant, there is no normal precedent. In double heart switched organ transplantation to add a second heart, that native will remain fully innervated while that added will be fully denervated in relation to the new body.

In double heart switch transplantation that involves replacing the native as well as adding a second heart, the orthotopically positioned heart will be partially, and that heterotopically positioned, entirely denervated with no corresponding nerve to allow reconnection in the recipient. Nervous recovery of the orthotopically transplanted heart takes about five years, but a second heart must be positioned heterotopically (see, for example, Stover, E. P. and Siegel. L. C. 1995. “Physiology of the Transplanted Heart,” International Anesthesiology Clinics 33(2): 11-20; Gaer, J. 1992. “Physiological Consequences of Complete Cardiac Denervation,” British Journal of Hospital Medicine 48(5): 220-225). Whether a second heart placed to function much as a living assist device need respond to stimuli such as exertional or emotional when the native heart remains neurologically and endocrinologically integrated for such response warrants study.

The unclear status in neurological recovery of even a patient with one heart conventionally transplanted orthotopically complicates the application of anesthesia in various surgical procedures before the question of a second heart even arises (Rabin, J. and Kaczorowski, D. J. 2019. “Perioperative Management of the Cardiac Transplant Recipient,” Critical Care Clinics 35(1): 45-60; Choudhury, M. 2017. Op cit. in this section; Herborn, J. and Parulkar, S. 2017. “Anesthetic Considerations in Transplant Recipients for Nontransplant Surgery,” Anesthesiology Clinics 35(3): 539-553; Jurgens, P. T., Aquilante, C. L., Page. R. L. 2nd, and Ambardekar, A. V. 2017. “Perioperative Management of Cardiac Transplant Recipients Undergoing Noncardiac Surgery: Unique Challenges Created by Advancements in Care,” Seminars in Cardiothoracic and Vascular Anesthesia 21(3): 235-244; Inoue. M., Hayashi, Y., Fujita, Y., Shimizu, M., Hotta, A., and 4 others 2016. “A Case of General Anesthesia for a Cardiac Transplanted Patient Undergoing Inguinal Hernia Repair under Laparoscopic Surgery,” (in Japanese with abstract at Pubmed), Masui. The Japanese Journal of Anesthesiology (Tokyo, Japan) 65(4): 359-362; Nassirou, O. M., Jaafari, A., Chlouchi, A., Bensghir, M., and Haimeur, C. 2016. “Anaesthesia for Noncardiac Surgery in a Heart-transplanted Patient: A Clinical Case and Review of the Literature,” (in French with English abstract and full text with translate feature at Pubmed), Pan African Medical Journal (Kampala, Uganda) 24:284; Pandya, S. R. and Paranjape. S. 2014. “Laparoscopic Cholecystectomy in a Cardiac Transplant Recipient,” Saudi Journal of Anaesthesia 8(2): 287-289; Valerio, R. Jr., Durra, O., and Gold, M. E. 2014. “Anesthetic Considerations for An Adult Heart Transplant Recipient Undergoing Noncardiac Surgery: A Case Report,” American Association of Nurse Anesthetists Journal 82(4): 293-299; Swami, A. C., Kumar, A., Rupal. S., and Lata, S. 2011. “Anaesthesia for Non-cardiac Surgery in a Cardiac Transplant Recipient,” Indian Journal of Anaesthesia 55(4): 405-407). For double heart transplantation, the answers must await the accumulation of experience.

“Mechanical ventilation can initiate ventilator-associated lung injury (VALI) and contribute to the development of multiple organ dysfunction. Although a lung protective strategy limiting both tidal volume and plateau pressure reduces VALI, uneven intrapulmonary gas distribution is still capable of increasing regional stress and strain, especially in non-homogeneous lungs, such as during acute respiratory distress syndrome. Real-time monitoring of regional ventilation may prevent inhomogeneous ventilation, leading to a reduction in VALI,” (Shono, A. and Kotani, T. 2019. “Clinical Implication of Monitoring Regional Ventilation Using Electrical Impedance Tomography,” Online, BioMed Central Journal of Intensive Care (London, England) 7:4).

Used inconsistently in the literature, here the terms ‘spontaneous ventilation,’ ‘spontaneous respiration,’ and ‘spontaneous breathing’ are used to denote normal unassisted breathing. Usually ‘spontaneous ventilation’ under general anesthesia does involve mechanical assistance, while the same absent general anesthesia does not. Switch transplantation, for example, usually eliminates the need for general anesthesia, and use of a surgical chest dome as described herein is intended to enable spontaneous breathing with general anesthesia eliminated.

A valid need for mechanical ventilatory assistance independently of—that is, during or not during—general anesthesia however, is not discounted (Ball, L., Costantino, F., Fiorito, M., Amodio, S., and Pelosi. P. 2018. “Respiratory Mechanics during General Anaesthesia,” Annals of Translational Medicine 6(19): 379). Although seldom allowed. “Spontaneous ventilation [here denoting self- initiated breathing with mechanical assistance] during general anesthesia has been shown to favor atelectasis formation and decreased functional residual capacity,” more recent developments having dispelled this problem (Magnusson, L. 2010. “Role of Spontaneous and Assisted Ventilation during General Anaesthesia,” Best Practice and Research. Clinical Anesthesiology 24(2): 243-252).

Most often, genuinely spontaneous, or normal, breathing during surgery is possible and is associated with the avoidance of adverse complications. Normal breathing is less susceptible to an adverse distribution of ventilation within the lungs (see, for example, Bauer, M., Opitz, A., Filser, J., Jansen, H., Meffert. R. H., and 4 others 2019. “Perioperative Redistribution of Regional Ventilation and Pulmonary Function: A Prospective Observational Study in Two Cohorts of Patients at Risk for Postoperative Pulmonary Complications,” Online, BioMed Central Anesthesiology (London, England) 19(1): 132; Cruces. P., Erranz. B., Lillo. F., Sarabia-Vallejos. M. A., Iturrieta, P., and 5 others 2019. “Mapping Regional Strain in Anesthetised Healthy Subjects during Spontaneous Ventilation,” Online, British Medical Journal Open Respiratory Research 6(1): e000423; Lagier, D., Fischer, F., Fornier, W., Huynh, T. M., Cholley, B., and 20 others 2019. “Effect of Open- lung vs Conventional Perioperative Ventilation Strategies on Postoperative Pulmonary Complications after On-pump Cardiac Surgery: The PROVECS [perioperative open-lung protective ventilation in cardiac surgery] Randomized Clinical Trial,” Intensive Care Medicine 45(10): 1401-1412; Putensen, C., Hentze, B., Muenster, S., and Muders. T. 2019. “Electrical Impedance Tomography for Cardio-pulmonary Monitoring,” Online, MDPI [Multidisciplinary Digital Publishing Institute] Journal of Clinical Medicine (Basel, Switzerland) 8(8). pii: E1176; Lagier, D., Fischer, F., Fornier, W., Fellahi, J. L., Colson, P., and 4 others with 32 collaborators 2018. “A Perioperative Surgeon-controlled Open-lung Approach versus Conventional Protective Ventilation with Low Positive End-expiratory Pressure in Cardiac Surgery with Cardiopulmonary Bypass (PROVECS) [perioperative open-lung protective ventilation in cardiac surgery]: Study Protocol for a Randomized Controlled Trial,” Trials 19(1): 624; Serpa Neto. A., Schultz, M. J., and Gama de Abreu, M. 2015. “Intraoperative Ventilation Strategies to Prevent Postoperative Pulmonary Complications: Systematic Review, Meta-analysis, and Trial Sequential Analysis,” Best practice and Research. Clinical Anesthesiology 29(3): 331-340; Severgnini, P., Selmo, G., Lanza, C., Chiesa, A., Frigerio, A., and 10 others 2013. “Protective Mechanical Ventilation during General Anesthesia for Open Abdominal Surgery Improves Postoperative Pulmonary Function,” Anesthesiology 118(6): 1307-1321; Radke, O. C., Schneider, T., Heller, A. R., and Koch, T. 2012. “Spontaneous Breathing during General Anesthesia Prevents the Ventral Redistribution of Ventilation as Detected by Electrical Impedance Tomography: A Randomized Trial,” Anesthesiology 116(6): 1227-1234; Hong, C. M., Xu, D. Z., Lu, Q., Cheng. Y., Pisarenko. V., and 6 others 2010. “Low Tidal Volume and High Positive End-expiratory Pressure Mechanical Ventilation Results in Increased Inflammation and Ventilator-associated Lung Injury in Normal Lungs,” Anesthesia and Analgesia 110(6): 1652-1660).

Similar questions arise in relation to perioperative anesthetic management following transplantation of other organs, all transplantable using the compound vascular bypass, or switch method, often with the aid of a surgical chest dome to avoid a remaining source of complications in in the form of mechanical rather than spontaneous respiration (normal breathing, normal ventilation) as addressed in this section, and below in section 2b(2)(k), entitled Surgical Chest Dome, and section 6, entitled Description of the Preferred Embodiments of the Invention (see, for example, Ersoy, Z., Ayhan, A., Ozdemirkan, A., Polat, G. G., Zeyneloglu P., Arslan, G., and Haberal. M. 2017. “Anesthetic and Perioperative Management of Nontransplant Surgery in Patients after Liver Transplant,” Experimental and Clinical Transplantation 15 (Supplement 1): 42-45; Ersoy, Z., Kaplan, S., Ozdemirkan, A., Torgay, A., Arslan, G., Pirat. A., and Haberal, M. 2017. “Effect of Graft Weight to Recipient Body Weight Ratio on Hemodynamic and Metabolic Parameters in Pediatric Liver Transplant: A Retrospective Analysis,” Experimental and Clinical Transplantation 15 (Supplement 1): 53-56; Casale, J. P. and Doligalski, C. T. 2016. “Pharmacologic Considerations for Solid Organ Transplant Recipients Who Become Pregnant,” Pharmacotherapy 36(9): 971-982; Moaveni, D. M., Cohn, J. H., Hoctor, K. G., Longman, R. E., and Ranasinghe, J. S. 2016. “Anesthetic Considerations for the Parturient after Solid Organ Transplantation,” Anesthesia and Analgesia 123(2): 402-410; Camkιran, A., Araz, C., Seyhan Ballι, S., Torgay, A., Moray, G., and 3 others 2014. “Anesthetic Management in Pediatric Orthotopic Liver Transplant for Fulminant Hepatic Failure and End-stage Liver Disease,” Experimental and Clinical Transplantation 12 Supplement 1:106-109; Seo. M., Kim, W. J., and Choi, I. C. 2014. “Anesthesia for Non-pulmonary Surgical Intervention following Lung Transplantation: Two Cases Report,” Korean Journal of Anesthesiology 66(4): 322-326; Feltracco, P., Falasco, G., Barbieri, S., Milevoj. M., Serra, E., and Ori, C. 2011. “Anesthetic Considerations for Nontransplant Procedures in Lung Transplant Patients,” Journal of Clinical Anesthesia 23(6): 508-516; Kostopanagiotou, G., Sidiropoulou, T., Pyrsopoulos, N., Pretto, E. A. Jr., Pandazi, A., and 4 others 2008. “Anesthetic and Perioperative Management of Intestinal and Multivisceral Allograft Recipient in Nontransplant Surgery,” Transplant International 21(5): 415-427; Zeyneloglu, P., Pirat, A., Sulemanii, D., Torgay, A., Karakayali, H., and Arslan, G. 2007. “Perioperative Anesthetic Management for Recipients of Orthotopic Liver Transplant Undergoing Nontransplant Surgery,” Experimental and Clinical Transplantation 5(2): 690-692; Feng, Z. Y., Zhang, J., Zhu, S. M., and Zheng, S. S. 2006. “Is There Any Difference in Anesthetic Management of Different Post-OLT [orthotopic liver transplant] Stage Patients Undergoing Nontransplant Organ Surgery?,” Hepatobiliary and Pancreatic Diseases International 5(3): 368-373; Kostopanagiotou, G., Smyrniotis, V., Arkadopoulos, N., Contis, J., Briassoulis, G., and Kostopanagiotou, E. 2003. “Anaesthetic and Perioperative Management of Paediatric Organ Recipients in Nontransplant Surgery,” Paediatric Anaesthesia 13(9): 754-763).

In double heart switch transplantation, the prescription-program will continue the advancement of the diversion servovalves until the implanted cardiac rhythm sensors indicate the inception of a dysrhythmia, which the program will have been written to reverse through electrical, temperature, pharmaceutical, and it can be predicted, optogenetic means (Guru, A., Post. R. J., Ho, Y. Y., and Warden, M. R. 2015. “Making Sense of Optogenetics,” International Journal of Neuropsychopharmacology 18(11):pyv079; references on optogenetics from the standpoint of implants with internal rechargeable power storage replenishable through transcutaneous power transfer are provided above in this section).

Once the native organ is removed, the graft organ is placed in the recipient with the valves and bloodlines connected to the graft organ and to the implanted microcontroller which had administered the operation. Thereafter, the controller, provided with data from implanted sensors, monitors and is able to target medication to the transplant from implanted small flat drug reservoirs connected to accessory channels in each valve jacket, can adjust the valves to regulate or fine tune the blood pressure, and can apply electrostimulation in accordance with the controller prescription-program.

Without accessory channels, common catheteric materials used as mainlines, bloodlines, central lines, vascular accesses, and urinary catheters would be susceptible to the buildup of thrombus, and/or biofilm, or crystal respectively to increase the risk of obstruction and/or infection (see, for example, Bouhrour, N., Nibbering, P. H., and Bendali, F. 2024. “Medical Device-associated Biofilm Infections and Multidrug-resistant Pathogens,” Online, MDPI [Multidisciplinary Digital Publishing Institute] Pathogens (Basel, Switzerland) 13(5): 393; Kanti, S. P. Y., Mukhtar, M., Cseh, M., Orosz, L., Burian, K., and 3 others 2024. “Investigation of Chlorhexidine and Chitosan Gel-based Coatings for the Prevention of Intravascular Catheter-associated Infections following Quality by Design Approach,” Online, MDPI [Multidisciplinary Digital Publishing Institute] Biomedicines (Basel, Switzerland) 12(9): 2032; Pitiriga. V. C., Campos. E., Bakalis. J., Saroglou, G., and Tsakris. A. 2024. “Differences in the Dwell Time of Peripherally Inserted Central Catheters between Patients with Catheter Colonization and Those Developing Central Line-associated Bloodstream Infection: A Single Centre Retrospective Cohort Study,” Online, MDPI [Multidisciplinary Digital Publishing Institute] Antibiotics (Basel, Switzerland) 13(7): 632; Haider; M. Z. and Annamaraju, P. 2023. “Bladder Catheterization,” Online, Treasure Island, Florida: StatPearls Publishing Co.; Kanti, S. P. Y., Csóka, I., Jójárt-Laczkovich, O., and Adalbert. L. 2022. “Recent Advances in Antimicrobial Coatings and Material Modification Strategies for Preventing Urinary Catheter-Associated Complications,” Online, MDPI [Multidisciplinary Digital Publishing Institute] Biomedicines Antibiotics (Basel, Switzerland) 10(10): 2580; Wang, L., Jia. L., and Jiang, A. 2022. “Pathology of Catheter-related Complications: What We Need to Know and What Should be Discovered” Online, Journal of International Medical Research (London, England) 50(10): 3000605221127890; Trautner, B. W. and Darouiche. R. O. 2004. “Catheter-associated Infections: Pathogenesis Affects Prevention,” Online, [American Medical Association] Archives of Internal Medicine (Chicago, Illinois) 164(8): 842-850; Marosok, R., Washburn, R., Indorf. A., Solomon, D., and Sherertz, R. 1996. “Contribution of Vascular Catheter Material to the Pathogenesis of Infection: Depletion of Complement by Silicone Elastomer in Vitro,” Journal of Biomedical Materials Research 1996 30(2): 245-250; Sherertz, R. J., Carruth, W. A., Marosok, R. D., Espeland, M. A., Johnson, R. A., and Solomon, D. D. 1995. “Contribution of Vascular Catheter Material to the Pathogenesis of Infection: The Enhanced Risk of Silicone in Vivo,” Journal of Biomedical Materials Research 29(5): 635-645; Curelaru. I., Gustavsson, B., Hultman, E., Jondmundsson, E., Linder, L. E., Stefansson, T., and Stenqvist, O. 1984. “Material Thrombogenicity in Central Venous Catheterization III. A Comparison Between Soft Polyvinylchloride and Soft Polyurethane Elastomer, Long, Antebrachial Catheters,” Acta Anaesthesiologica Scandinavica 28(2): 204-208). INSERT OTHER adverse tissue provoking CATHETER MATERIALS HERE

This automatic post-transplantation maintenance, which can instantly provide an immunosuppressant or anti-inflammatory, for example, can not only prevent graft organ failure but make possible organ transplants not previously considered, alleviating the shortage of replacement organs as but one of several benefits. With respect to long-term synthetic-to-native tissue ductus junctions whether end-to-end, end-to-side anastomotic, or those involving synthetic grafts such as a woven polyethylene (Dacron®, DuPont deNemours, Wilmington, Delaware) nitinol reinforced patch on the bladder, in a Dor endoventricular circular patch plasty on the left ventricle, or in a vascular bypass such as femorofemoral shunt, for example, absent immediately responsive protective measures, adverse tissue reactions, infection, and the accumulation of clot often prove problematic (see, for example, Spadaccio, C., Rainer, A., Barbato, R., Trombetta, M., Chello, M., and Meyns. B. 2019. “The Long-term Follow-up of Large-diameter Dacron® Vascular Grafts in Surgical Practice: A Review,” Journal of Cardiovascular Surgery (Turin, Italy) 60(4): 501-513; U.S. Food and Drug Administration 2019. “Class 2 Device Recall HEMASHIELD PLATINUM Woven Double Velour Vascular Graft,” Online at https://www.accessdata.fda.gov/scripts/cdrh/cfdocs/cfRes/res.cfm?id=176040; Spadaccio, C., Nappi, F., Al-Attar, N., Sutherland, F. W., Acar, C., and 4 others 2016. “Old Myths, New Concerns: The Long-Term Effects of Ascending Aorta Replacement with Dacron Grafts. Not All that Glitters is Gold,” Journal of Cardiovascular Translational Research 9(4): 334-342; Ugurlucan M., Oztas, D. M., Onal, Y., Canbay, C., Sayin, O. A., and 6 others 2016. “Treatment of Dacron Grafting Dilatation with Endovascular Stent-grafting,” Aorta (Stamford, Connecticut) 4(5): 162-166).

Older conventional synthetic patches may also allow blood, especially heparinized, to seep through the material, wherefor aneurysm and/or coarctation bypass conduits addressed below in the section of like title are made of a new triple layered material (De Paulis. R., Scaffa. R., Maselli, D., Salica. A., Bellisario. A., and Weltert, L. 2008. Op cit. above in this section). The ability afforded by the accessory channels in ductus side-entry jackets or nonjacketing side-entry connectors to target anti-inflammatory, antithrombotic, and antimicrobial medication to these junctions is a powerful deterrent to junction failure.

The thrombotic, infection, and adverse tissue reaction problems associated with Dacron® and similar synthetic textiles in direct contact with tissue can be significantly ameliorated if not eliminated. Patches and shunts, especially if supported as indicated, are effective for repairing defects in the circulatory system that would otherwise be fatal for at least a few years, making use in the elderly satisfactory. Prevention of another junction-related complication, perigraft seroma, is by conventional methods for eliminating spaces where fluid can accumulate (Halloul, Z., Rimpler, H., Waliszewski, M., Beier, N., Meyer, F., ad 3 others 2014. “First-in-Man Results of a Novel Vascular Graft Coated with Resorbable Polymer for Aortic Reconstructions—a Multicenter, Non-randomized Safety Study,” Langenbeck's Archives of Surgery 399(5): 629-638; Rhodes, V. J. 1986. “Perigraft Seroma: Simple Solution to a Difficult Problem,” Journal of Vascular Surgery (New York, New York) 36): 389).

The fabric conventionally used in small children the same as that used in adults, unless replaced in a second painful operation, a woven polyester stitched patch to serve as an aortoplasty along the thoracic aorta, unable to accommodate more than negligible growth, the patch will first restrict normal growth and eventually leak leading to the formation of a seroma if not a rupture risking death. Neither woven nor stitched, use of the expandable structure for fluid conveying lines in tiny patients described below in section 2b(2)(f), entitled Expandable Structure to Accommodate Growth and Self-adjust in Size in Vascular Segment Prostheses, Lines, and Confluence Conduits Placed in Children, eliminates any such eventuality.

Nevertheless, out of an ‘abundance of caution,’ marketed aortic prostheses can be coated in thin layer of stretchable parylene and any potential ‘blind’ spaces, that could fill with serous fluid should be provided with drains ab initio (see, for example, Doita, T., Yamasumi, T., Nakamura, Y, and Kondoh, H. 2023. “Subcutaneous Extension of Perigraft Seroma after Thoracic Aortic Surgery-Report of Three Cases,” Online, General Thoracic and Cardiovascular Surgery (Tokyo, Japan) 2, 8 [in press]; Kanemitsu, S., Sakamoto, S., Teranishi, S., and Mizumoto, T. 2022. “Expanding Perigraft Seroma after Ascending Aorta Replacement,” Online, BioMed Central (London, England) Journal of Cardiothoracic Surgery 17(1): 252; Kunibe, Y., Ando, M., Komae, H., Shimada, S., Kinoshita, O., Yamauchi, H. and Ono, M. 2022. “Perigraft Seroma after Total Aortic Arch Replacement Using Triplex Graft,” General Thoracic and Cardiovascular Surgery (Tokyo, Japan) 70(7): 668-672; Broda, M., Budtz-Lilly, J., Vammen, S. and Pedersen, S. F. 2019. “Perigraft Seroma Penetrating the Aortic Sac and Rupturing into the Intraperitoneal Cavity,” Online, Journal of Vascular Surgery Cases and Innovative Techniques (New York, New York) 5(3): 298-301; Yamauchi T., Kubota, S., Hagawa, K., and Suzuki, A. 2017. “Mediastinal Perigraft Seroma after Repair of Type A Dissection with a Polyester Graft,” Online, Annals of Vascular Diseases (Tokyo, Japan) 10(1): 51-53).

Yet other junctional or interface contact-related problems result from the use of polymer surface coatings on catheters and other implant devices; such problems are identified with intracranial procedures where access is difficult, as well as in other contexts (see, for example, Sabzevari, N., Schapiro, B. L., and Stewart, B. 2019. “Necrotic Leg Ulcers Secondary to Hydrophilic Polymer Gel Emboli,” Journal of Vascular Surgery Cases and Innovative Techniques (New York, New York) 5(3): 310-313; Chopra, A. M., Mehta, M., Bismuth, J., Shapiro, M., Fishbein, M. C., Bridges, A. G., and Vinters, H. V. 2017. “Polymer Coating Embolism from Intravascular Medical Devices—A Clinical Literature Review,” Cardiovascular Pathology 30:45-54; Mehta, R. I. and Mehta, R. I. 2016. “Polymer-induced Central Nervous System Complications following Vascular Procedures: Spectrum of Iatrogenic Injuries and Review of Outcomes,” Human Pathology 53:178-190; Thompson, A. K., Peters, M. S., El-Azhary, R. A., Gibson, L. E., Chang, M. B., and 5 others 2015. “Cutaneous Microemboli from Hydrophilic Polymer after Endovascular Procedures,” Journal of the American Academy of Dermatology 73(4): 666-671; Rosen, L. E., Singh, R. I., and Mahon, B. 2014. “Myocardial Hydrophilic Polymer Emboli following Cardiac Catheterization: A Case Report and Literature Review,” Cardiovascular Pathology 23(3): 175-177).

A similar postoperative complication is a perigraft hygroma pertains to expanded polytetrafluoroethylene as well as woven polyester (King, A. H., Ambani, R. N., Harth, K. C., Kashyap, V. S, Kumins, N. J., and 3 others 2020. “Frequency of Perigraft Hygroma after Open Aortic Reconstruction,” Online, Journal of Vascular Surgery (New York, New York) 72(1): 154-161). Usually used to denote a pocket of fluid due to overuse injury in a joint seen in veterinary practice, here the term “hygroma” is used as synonymous with seroma.

In some cases, particles of the coating shed due to sliding contact during the insertion process result in an inflammatory response, arterial pathology, or embolism, for example, long after the procedure when the coated insertion device had been withdrawn. While in extracranial applications, immunomodulatory therapy with mycophenolate, azathioprine, and steroids can be targeted to the site through the accessory channels, these too can produce serious side effects, leaving the problem best avoided by not using polymer coatings such as polyvinylpyrrolidone, mostly if not all hydrophilic, implicated thus in the first place. No such coatings, which rub off the substrate during insertion to remain behind as an irritant, are applied to side-entry jackets, nonjacketing side-entry connectors, side-entry diversion valves, diversion servovalves, chokevalves, or servo chokevalves.

The coatings, applied to obtain a slippery surface to avoid intravascular clinging, would best be replaced with a sputter deposited fluoropolymer, for example. However, because the nonliving, static material is incapable of the functional, restorative, and adaptive attributes of living vascular or bladder tissue, it is unable to reverse a loss in tautness, for example, and is subject to deterioration over the long-term, so that problems unrelated to the synthetic-native junction continue to interfere with its sufficiency for use in the young (see, for example, Singh, C., Wong, C. S., and Wang, X. 2015. “Medical Textiles a5s Vascular Implants and Their Success to Mimic Natural Arteries,” Journal of Functional Biomaterials 6(3): 500-525;

Ductus side-entry jackets, nonjacketing side-entry connectors, diversion jackets used as vascular servovalves, and servochokes incorporate features specifically intended to overcome the factors that historically precluded the permanent implantation of catheters and smaller gauge tubing of a material such as Dacron® to move blood or urine. Accessory channels giving access through a small port at the body surface are provided not only to allow the direct targeting to the treatment site of medication, but to wet the interior surface of lines with maintenance materials to counteract thrombus in the vascular tree, crystallization in the urinary tract, and the risk of biofilm and infection.

Whereas a diversion jacket redirects all flow through a vessel, ureter, spermatic duct, fallopian tube, glandular duct, or other tubular anatomical structure, the diversion of arterial blood usually seeks to redirect only a fraction of the blood moving through the artery to perfuse an under-oxygenated region of organ. Partial diversion is accomplished with a conventional side-entry jacket used to create a branch of the main vessel.

To prevent the buildup of thrombus, crystallization, and/or biofilm as deterrents to the use on a permanent, fully implanted, basis of synthetic materials and smaller caliber catheters in particular, a special port at the body surface having openings to allow the direct delivery through the jacket or connector accessory channel or channels into the synthetic lumen, side-entry jacket, and native lumen of coagulation, crystallization, and biofilm forming counteractants as necessary are injected into the small flat drug reservoir or reservoirs implanted subcutaneously in the pectoral region.

The permanent implantation of catheters by the means described herein with subcutaneously positioned body surface port which can be made unnoticeable, allows the use of larger caliber catheters than does brachial, jugular, subclavian, and even vena cava insertion, for example (Fonseca, I. Y., Krutman, M., Nishinari, K., Yazbek, G., Teivelis, M. P., Bomfim, G. A., Cavalcante, R. N., and Wolosker, N. 2016. “Brachial Insertion of Fully Implantable Venous Catheters for Chemotherapy: Complications and Quality of Life Assessment in 35 Patients,” Einstein (Sao Paulo, Brazil) 14(4): 473-479).

Furthermore, the connection to the ductus of the catheter through the side-entry jacket is accomplished without endoluminal, or intravascular, entry, and the type of port described here is less susceptible to complications, to include infection, fracture, or migration (Ko, S. Y., Park, S. C., Hwang, J. K., and Kim, S. D. 2016. “Spontaneous Fracture and Migration of Catheter of a Totally Implantable Venous Access Port Via Internal Jugular Vein-A Case Report,” Journal of Cardiothoracic Surgery 11:50; Pignataro, B. S., Nishinari, K., Wolosker, N., and Bomfim, G. A. 2014. “Fracture and Migration into the Coronary Sinus of a Totally Implantable Catheter Introduced via the Right Internal Jugular Vein” Online, BMJ [British Medical Journal] Case Reports 2014 pii: bcr2014207276).

The minuscule side plug extraction method for gaining endoluminal entry into the substrate ductus is less susceptible to complications such as the inducement of clotting or infection, than alternative methods. (Hsu, C. C., Kwan, G. N., Evans-Barns, H., Rophael, J. A., and van Driel, M. L. 2016. “Venous Cutdown Versus the Seldinger Technique for Placement of Totally Implantable Venous Access Ports,” Cochrane Database of Systematic Reviews(8): CD008942; Klaiber, U., Grummich, K., Jensen, K., Saure, D., Contin, P., and 3 others 2015. “Closed Cannulation of Subclavian Vein vs Open Cut-down of Cephalic Vein for Totally Implantable Venous Access Port (TIVAP) Implantation: Protocol for a Systematic Review and Proportional Meta-analysis of Perioperative and Postoperative Complications,” Systematic Reviews 4:53; Orci, L. A., Meier, R. P., Morel, P., Staszewicz, W., and Toso, C. 2014. “Systematic Review and Meta-analysis of Percutaneous Subclavian Vein Puncture Versus Surgical Venous Cutdown for the Insertion of a Totally Implantable Venous Access Device,” British Journal of Surgery 101(2): 8-16; Jablon, L. K., Ugolini, K. R., and Nahmias, N. C. 2006. “Cephalic Vein Cut-down verses Percutaneous Access: A Retrospective Study of Complications of Implantable Venous Access Devices,” American Journal of Surgery 192(1): 63-67).

For example, two hearts with marginal ventricular absolute stroke volumes (ejection fractions) were these measured while the donor heart also remained in its respective circulatory system (Konstam, M. A. and Abboud, F. M. 2017. Op cit.) would fail to meet the current criteria for transplantation. However, using metered double heart transplantation as addressed below in section 2b(1)(b)(b5), entitled Heterotopic Double Heart Metered Compound Vascular Bypass, or Switched-organ, Transplantation [and so on], can be coordinated to approximate the function of a normal heart, and if the need is detected by the implanted response system sensors, either or both hearts can be directly pipeline-targeted supportive medication and/or electrostimulation postoperatively for as long as necessary.

Evaluating the potential for reverse remodeling and recovery of a marginally impaired replacement heart once transferred from the circulatory system of the deceased prospective donor on life support into that of the recipient must be based upon the detailed pathology of both and the graft organ. Such an evaluation draws upon all aspects of cardiology and much of internal medicine. To this must be added the complexity of weighing these factors against the relative merits and risks posed by conventional and metered switch orthotopic transplantation or a double heart transplantation whereby the graft is implanted as a second heart.

The donor, graft organ, recipient, and probable consequences of different courses of action must be taken into account. Then post-transplantation care requires detailed familiarity with cardiology and cardiological pharmacotherapeutics, and if a need therefor had not been realized in advance, this must include the use of various ventricular assist devices and/or implanted direct stimulatory means both as mentioned above and/or in the form of conventional pacemaker/cardioverter defibrillators in relation to the detailed condition of the recipient. Comorbidity if not intercurrent disease is likely to further complicate matters, potentially demanding a knowledge of almost any aspect of internal medicine.

Cardiac remodeling following a myocardial infarction is not unique to reduced ejection fraction; remodeling can lead to reduced, midrange, or preserved ejection fraction. Neither is pulmonary hypertension exclusively associated with remodeling of the right or left ventricle or are cause or effect necessarily clear as to right or left heart dysfunction (see, for example, Borlaug. B. A. and Reddy. Y. N. V. 2019. “The Role of the Pericardium in Heart Failure: Implications for Pathophysiology and Treatment,” Journal of the American College of Cardiology. Heart Failure 7(7): 574-585; Kheyfets. V., Truong. U., Ivy. D., and Shandas. R. 2019. “Structural and Biomechanical Adaptations of Right Ventricular Remodeling—In Pulmonary Arterial Hypertension—Reduces Left Ventricular Rotation during Contraction: A Computational Study,” Journal of Biomechanical Engineering February 4; Kido, K. and Coons, J. C. 2019. “Efficacy and Safety of the Use of Pulmonary Arterial Hypertension Pharmacotherapy in Patients with Pulmonary Hypertension Secondary to Left Heart Disease: A Systematic Review,” Pharmacotherapy 39(9): 929-945; Kwon, H. J., Park. J. H., Park, J. J., Lee, J. H., and Seong, I. W. 2019. “Improvement of Left Ventricular Ejection Fraction and Pulmonary Hypertension are Significant Prognostic Factors in Heart Failure with Reduced Ejection Fraction Patients,” Journal of Cardiovascular Imaging 27(4): 257-265; Martinez, C., Tsugu, T., Sugimoto. T., and Lancellotti, P. 2019. “Pulmonary Hypertension with Valvular Heart Disease: When to Treat the Valve Disease and When to Treat the Pulmonary Hypertension,” Current Cardiology Reports 21(12): 151; Rao, S. D., Menachem, J. N., Birati, E. Y., and Mazurek, J. A. 2019. “Pulmonary Hypertension in Advanced Heart Failure: Assessment and Management of the Failing RV and LV,” Current Heart Failure Reports 16(5): 119-129; Rosenkranz, S., Kramer, T., Gerhardt. F., Opitz, C., Olsson, K. M., and Hoeper, M. M. 2019. “Pulmonary Hypertension in HFpEF and HFrEF: Pathophysiology, Diagnosis, Treatment Approaches,” Her: 44(6): 483-490; Ren X., Johns. R. A., and Gao, W. D. 2019. “Right Heart in Pulmonary Hypertension: From Adaptation to Failure,” Pulmonary Circulation 9(3) 1-20; Simon, J., Nemeth, E., Nemes, A., Husveth-Toth, M., Radovits. T., and 6 others 2019. “Circulating Relaxin-1 Level is a Surrogate Marker of Myocardial Fibrosis in HFrEF [heart failure with reduced ejection fraction],” Online, Frontiers in Physiology 10: 690; Tackling, G. and Borhade. M. B. 2019. “Hypertensive Heart Disease,” Online, Treasure Island, Florida: StatPearls Publishing; Charalampopoulos, A., Lewis, R., Hickey, P., Durrington, C., Elliot. C., and 3 others 2018. “Pathophysiology and Diagnosis of Pulmonary Hypertension Due to Left Heart Disease,” Online, Frontiers in Medicine (Lausanne, Switzerland) 5:174; Adusumalli, S. and Mazurek, J. A. 2017. “Pulmonary Hypertension Due to Left Ventricular Cardiomyopathy: Is it the Result or Cause of Disease Progression?,” Current Heart Failure Reports 14(6): 507-513; Platt. M. J., Huber, J. S., Romanova, N., Brunt, K. R., and Simpson, J. A. 2018. “Pathophysiological Mapping of Experimental Heart Failure: Left and Right Ventricular Remodeling in Transverse Aortic Constriction is Temporally, Kinetically, and Structurally Distinct,” Online, Frontiers in Physiology 9:472; Bosch, L., Lam, C. S. P., Gong. L., Chan, S. P., Sim, D., and 8 others 2017. “Right Ventricular Dysfunction in Left-sided Heart Failure with Preserved versus Reduced Ejection Fraction,” European Journal of Heart Failure 19(12): 1664-1671; Ghio, S., Guazzi, M., Scardovi. A. B., Klersy, C., Clemenza, F., and 7 others 2017. “Different Correlates but Similar Prognostic Implications for Right Ventricular Dysfunction in Heart Failure Patients with Reduced or Preserved Ejection Fraction,” European Journal of Heart Failure 19(7): 873-879; Rosenkranz, S., Gibbs, J. S., Wachter. R., De Marco, T., Vonk-Noordegraaf. A., and Vachiery, J. L. 2016. “Left Ventricular Heart Failure and Pulmonary Hypertension,” European Heart Journal 37(12): 942-954; Vonk-Noordegraaf, A., Haddad, F., Chin, K. M., Forfia, P. R., Kawut, S. M., and 8 others 2013. “Right Heart Adaptation to Pulmonary Arterial Hypertension: Physiology and Pathobiology,” Journal of the American College of Cardiology 62(25 Supplement): D22-D33). More generally, in heart failure, the major features-premonitory remodeling, right or left heart dysfunction, reduced, midrange, or preserved ejection fraction, right or left ventricular hypertension, and so on, present in all combinations to present as independent variables. Furthermore, the interrelations of each type disorder to the other organs comprises a considerable profusion of combinations and permutations.

Hearts that had undergone a myocardial insult, typically, a myocardial infarction, likely to induce remodeling leading to heart failure can be evaluated for such a prognosis (Stoll. V. M., Hess. A. T., Rodgers, C. T., Bissell, M. M., Dyverfeldt. P., and 4 others 2019. “Left Ventricular Flow Analysis: Novel Imaging Biomarkers and Predictors of Exercise Capacity in Heart Failure,” Circulation. Cardiovascular Imaging 12(5): e008130)(see also, for example, Berlot. B., Moya Mur, J. L., Jug. B., Rodriguez Muñoz, D., Megias, A., and 3 others 2019. “Effect of Diastolic Dysfunction on Intraventricular Velocity Behavior in Early Diastole by Flow Mapping,” International Journal of Cardiovascular Imaging 35(9): 1627-1636; Mangion, K., McComb, C., Auger, D. A., Epstein, F. H., and Berry, C. 2017. “Magnetic Resonance Imaging of Myocardial Strain after Acute ST-segment-elevation Myocardial Infarction: A Systematic Review,” Circulation. Cardiovascular Imaging 10(8). pii: e006498; Shah, A. M. 2013. “Ventricular Remodeling in Heart Failure with Preserved Ejection Fraction,” Current Heart Failure Reports 10(4): 341-349).

Means available and under development exist to monitor and suppress adverse remodeling in heart failure with reduced ejection fraction (see, for example, Arendse. L. B., Danser, A. H. J., Poglitsch, M., Touyz, R. M., Burnett, J. C. Jr., and 3 others 2019. “Novel Therapeutic Approaches Targeting the Renin-Angiotensin System and Associated Peptides in Hypertension and Heart Failure,” Pharmacological Reviews 71(4): 539-570; Bayard, G., Da Costa, A., Pierrard, R., Romeyer-Bouchard, C., Guichard, J. B., and Isaaz, K. 2019. “Impact of Sacubitril/Valsartan on Echo Parameters in Heart Failure Patients with Reduced Ejection Fraction. A Prospective Evaluation,” International Journal of Cardiology. Heart and Vasculature 25:100418; Burke, R. M., Lighthouse, J. K., Mickelsen, D. M., and Small, E. M. 2019. “Sacubitril/Valsartan Decreases Cardiac Fibrosis in Left Ventricle Pressure Overload by Restoring PKG [protein kinase G/cyclic guanosine monophosphate (cGMP)-dependent protein kinase] Signaling in Cardiac Fibroblasts,” Circulation. Heart Failure 12(4): e005565; De Vecchis. R., Ariano, C., Di Biase, G., and Noutsias, M. 2019. “Sacubitril/Valsartan for Heart Failure with Reduced Left Ventricular Ejection Fraction: A Retrospective Cohort Study,” Herz 44(5): 425-432; Hajra, A., Ujjawal, A., Sud, K., Chakraborty. S., and Bandyopadhyay. D. 2019. “Sacubitril/Valsartan Averts Post-myocardial Infarction Ventricular Remodeling and Preserves Heart Function,” International Journal of Cardiology. Heart and Vasculature (Shannon, Ireland) 22:218-219; Pfau, D., Thorn, S. L., Zhang, J., Mikush, N., Renaud, J. M., and 7 others 2019. “Angiotensin Receptor Neprilysin Inhibitor Attenuates Myocardial Remodeling and Improves Infarct Perfusion in Experimental Heart Failure,” (Nature Research) Scientific Reports 9(1): 5791; Velazquez, E. J., Morrow, D. A., De Vore, A. D., Duffy, C. I., Ambrosy, A. P., and 3 others 2019. “Angiotensin-Neprilysin Inhibition in Acute Decompensated Heart Failure,” New England Journal of Medicine 380(6): 539-548; Wang. Y., Guo, Z., Gao, Y., Liang, P., Shan, Y., and He, J. 2019. “Angiotensin II Receptor Blocker LCZ696 Attenuates Cardiac Remodeling through the Inhibition of the ERK [extracellular signal-regulated kinase] Signaling Pathway in Mice with Pregnancy-associated Cardiomyopathy,” Cell and Bioscience 9:86; Wang. Y., Zhou, R., Lu, C., Chen, Q., Xu. T., and Li, D. 2019. “Effects of the Angiotensin-receptor Neprilysin Inhibitor [sacubitril/valsartan] on Cardiac Reverse Remodeling: Meta-analysis,” Journal of the American Heart Association (Oxford, England) 8(13): e012272; Kario, K. 2018. “The Sacubitril/Valsartan, a First-in-class, Angiotensin Receptor Neprilysin Inhibitor (ARNI): Potential Uses in Hypertension, Heart Failure, and Beyond” Current Cardiology Reports 20(1): 5; Kompa, A. R., Lu, J., Weller, T. J., Kelly, D. J., Krum, H., von Lueder, T. G., and Wang, B. H. 2018. “Angiotensin Receptor Neprilysin Inhibition Provides Superior Cardioprotection Compared to Angiotensin Converting Enzyme Inhibition after Experimental Myocardial Infarction,” International Journal of Cardiology (Amsterdam, North Holland, Netherlands) 258: 192-198; Martens, P., Belien, H., Dupont, M., Vandervoort, P., and Mullens. W. 2018. “The Reverse Remodeling Response to Sacubitril/Valsartan Therapy in Heart Failure with Reduced Ejection Fraction,” Cardiovascular Therapeutics 36(4): e12435; Almufleh, A., Marbach, J., Chih, S., Stadnick, E., Davies, R., Liu, P., and Mielniczuk, L. 2017. “Ejection Fraction Improvement and Reverse Remodeling Achieved with Sacubitril/Valsartan in Heart Failure with Reduced Ejection Fraction Patients,” American Journal of Cardiovascular Disease 7(6): 108-113; Iborra-Egea, O., Galvez-Montón, C., Roura, S., Perea-Gil, I., Prat-Vidal. C., Soler-Botija, C., and Bayes-Genis, A. 2017. “Mechanisms of Action of Sacubitril/Valsartan on Cardiac Remodeling: A Systems Biology Approach,” Nature Partner Journals Systems Biology and Applications 3:12; Khder, Y., Shi, V., McMurray, J. J. V., and Lefkowitz, M. P. 2017. “Sacubitril/Valsartan (LCZ696) in Heart Failure,” Handbook of Experimental Pharmacology 243:133-165; Noble, M. I. 2017. “Whatever Happened to Measuring Ventricular Contractility in Heart Failure?,” Cardiac Failure Review 3(2): 79-82; Tyler, J. M. and Teerlink, J. R. 2017. “The Safety of Sacubitril-Valsartan for the Treatment of Chronic Heart Failure,” Expert Opinion on Drug Safety 16(2): 257-263; Zaid Iskandar, M. and Lang, C. C. 2017. “Sacubitril and Valsartan Fixed Combination to Reduce Heart Failure Events in Post-acute Myocardial Infarction Patients,” Drugs of Today (Barcelona, Spain) 53(10): 545-551; Azevedo, P. S., Polegato, B. F., Minicucci, M. F., Paiva, S. A., and Zomoff, L. A. 2016. “Cardiac Remodeling: Concepts, Clinical Impact, Pathophysiological Mechanisms and Pharmacologic Treatment,” Arquivos Brasileiros de Cardiologia (Brazilian Archives of Cardiology, Sao Paulo, Brazil) 106(1): 62-69; Waring, A. A. and Litwin, S. E. 2016. “Redefining Reverse Remodeling: Can Echocardiography Refine Our Ability to Assess Response to Heart Failure Treatments?,” Journal of the American College of Cardiology 68(12): 1277-1280).

Ventricular assist devices, conventional, and metered compound vascular bypass double heart heterotopic transplantation as addressed below in section 2b(1)(b)(b5), entitled Heterotopic Double Heart Metered Compound Vascular Bypass, or Switched-organ Transplantation [and so on], each have advantages and disadvantages. Vascular assist devices risk coagulopathy but involve the least surgical trauma to place. Conventional and double heart heterotopic transplantation should not cause a coagulopathy, but both require open chest surgery, will never be endoscopic, and the conventional heterotopic technique inflicts considerable surgical trauma upon both the recipient and donor hearts.

The reversal of pathology-adaptive remodeling with the use of a ventricular assist device and conventional heterotopic transplantation is infrequent and anticipated to be greater following a double heart transplant. The assist device is adjustable where the organic alternatives prompt adjustment by spontaneous adaptation over time. By allowing adjustment in the frequency of switching to every second or third successive systole of either heart, for example, double heart switch transplantation permits the effective ejection fraction to be controlled.

This factor allows compensating for the hypertension experienced by all transplant recipient within ten years following transplantation, as well as the renal insufficiency probably secondary thereto (Pandya, S. R. and Paranjape. S. 2014, Op cit. in this section). Moreover, by 1. Eliminating ischemia and 2. Accomplishing initial immune tolerance induction integral to the transferring of the transplant from the donor to the recipient, the switch procedure should reduce the severity if not the incidence of cardiac allograft vasculopathy

While in double heart transplantation the work of driving the blood is divided between two hearts assisted only by one another and drugs as appropriate in a reciprocal mutual assistance arrangement, a ventricular assist device supplements ventricular contractile function, partially if not entirely relieving the ventricular myocardium of exertion. The physiological indicia surrounding each include similarities and differences. Requiring to be substantiated through physiological testing, that following a double heart transplant, the left ventricles continue functioning but under a reduced load would seem equally conducive to recovery and reverse remodeling than the use of an optimally adjusted assist device and superior were the device adjusted to assume more of the work than necessary.

Double heart transplantation makes available an alternative to a ventricular assist device when the latter is contraindicated, especially due to its cumulative adverse effects in a child, or an orthotopic replacement of an adequate heart is precluded due to nonavailability in an exigent circumstance, or because the cost for a reasonably good heart is not prohibitively expensive compared to that for a mechanical assist device in that part of the world. Unless the need therefor could not have been known in advance, the intraoperative addition of an assist device to a double heart transplant will in some cases be discounted as an alternative to the need for a second heart. In parts of the world where surgery is less costly, a second heart that would otherwise be rejected is free and far more readily available than a healthy heart, so that double heart transplantation may be considered a ‘poor man's ventricular assist device.’

Assist devices for use in neonates and infants, to include those most recent, such as the Berlin Heart North America EXCOR® Pediatric. The Woodlands, Texas, are paracorporeal, that is, located outside the body, and an impediment to freedom of movement and the wearing of infant clothing, but in the absence of a viable alternative, life-saving (Shin, Y. R., Park, Y. H., and Park, H. K. 2019. “Pediatric Ventricular Assist Device,” Korean Circulation Journal 49(8): 678-690)—important due to the limited availability of infant hearts (see, for example, Dipchand, A. I. 2018. “Current State of Pediatric Cardiac Transplantation,” Annals of Cardiothoracic Surgery 7(1): 31-55; John, M. and Bailey, L. L. 2018. “Neonatal Heart Transplantation,” Annals of Cardiothoracic Surgery 7(1): 118-125).

“We conclude that heterotopic transplantation of a marginal donor heart can save an otherwise-dying orthotopic transplant recipient,” “Ejection fraction as indicated by echocardiography showed that the left ventricular function of the donor 1 heart was poor (LVEF, 0.30) at the beginning, but that it gradually recovered-under the support of the donor 2 heart—to normal function (LVEF, 0.62) 3 weeks later,” “This experience shows that the additional transplantation of a marginally acceptable donor heart in heterotopic position can save a patient who is dying of graft failure immediately after orthotopic heart transplantation,” (Chu, S. H., Chiu, K. M., Lin, T. Y., Chu, T. W., Yeih, D. F., and Li, A. H. 2007. “Two Donor Hearts Beat in One Chest,” Texas Heart Institute Journal 34(2): 230-232).

The cost for an assist device, surgery, and follow-up care in developed countries can amount to several hundred thousand dollars (see, for example, Tadmouri, A., Blomkvist, J., Landais, C., Seymour, J., and Azmoun, A. 2018. “Cost-effectiveness of Left Ventricular Assist Devices for Patients with End-stage Heart Failure: Analysis of the French Hospital Discharge Database,” European Society of Cardiology Heart Failure 5(1): 75-86; Baras Shreibati, J., Goldhaber-Fiebert, J. D., Banerjee, D., Owens, D. K., and Hlatky, M. A. 2017. “Cost-effectiveness of Left Ventricular Assist Devices in Ambulatory Patients with Advanced Heart Failure,” Journal of the American College of Cardiology. Heart Failure 5(2): 110-119; Chew, D. S., Manns, B., Miller, R. J. H., Sharma, N., and Exner, D. V. 2017. “Economic Evaluation of Left Ventricular Assist Devices for Patients with End Stage Heart Failure who are Ineligible for Cardiac Transplantation,” Canadian Journal of Cardiology 33(10): 1283-1291; Clarke, A., Pulikottil-Jacob, R., Connock, M., Suri, G., Kandala, N. B., and 3 others 2014. “Cost-effectiveness of Left Ventricular Assist Devices (LVADs) for Patients with Advanced Heart Failure: Analysis of the British NHS [national health service] Bridge to Transplant (BTT) Program,” International Journal of Cardiology 171(3): 338-345).

In countries with socialized medicine, making double heart transplantation-which tolerates hearts less robust than those acceptable for an orthotopic transplant—an effective alternative to assist devices would likely allow needy patients to receive care more promptly Unless having been confirmed to respond to therapy, hearts previously described as presenting diastolic heart failure, or informally, as showing normal contractility, but now referred to as heart failure with preserved ejection fraction, are unlikely to provide favorable transplantation outcomes, even in double heart transplantation as addressed below in section 2b(1)(b)(b5), so that representing more than half of all failing hearts notwithstanding, such hearts should be avoided.

Confirmed effective in the treatment of heart failure with reduced ejection fraction, sacubitril-valsartan has not offered comparable results in the treatment of heart failure with preserved ejection fraction (see, for example, Cinteza, M. 2019. “Chronic Heart Failure with Normal Contractility,” Medica (Bucharest, Romania) 14(3): 79-80; Nikolova, A. P., Hong. T., and Shaw, R. M. 2019. “Letter by Nikolova et al Regarding Article. “Heart Failure with Preserved Ejection Fraction in Perspective,” Circulation Research 125(4): e24-e25; Pfeffer, M. A., Shah, A. M., and Borlaug. B. A. 2019. “Heart Failure with Preserved Ejection Fraction in Perspective,” Circulation Research 124(11): 1598-1617; Pfeffer, M. A., Shah, A. M., and Borlaug, B. A. 2019. “Response by Pfeffer et al, to Letter Regarding Article, “Heart Failure With Preserved Ejection Fraction in Perspective,” Circulation Research 125(4): e26; Solomon, S. D., McMurray, J. J. V., Anand, I. S., Ge, J., Lam, C. S. P., and 29 others with 852 collaborators 2019. “Angiotensin-Neprilysin Inhibition in Heart Failure with Preserved Ejection Fraction,” New England Journal of Medicine 381(17): 1609-1620; Bajaj, N. S., Claggett. B., Lewis, E. F., Desai, A. S., Fang. J. C., and 9 others 2018. “Influence of Ejection Fraction on Cause-specific Mortality in Heart Failure with Preserved Ejection Fraction,” European Journal of Heart Failure 20(4): 815-816; Del Buono, M. G., Buckley, L., and Abbate, A. 2018. “Primary and Secondary Diastolic Dysfunction in Heart Failure with Preserved Ejection Fraction,” American Journal of Cardiology 122(9): 1578-1587; Dinatolo, E., Sciatti, E., Anker, M. S., Lombardi, C., Dasseni, N., and Metra, M. 2018. “Updates in Heart Failure: What Last Year Brought to Us,” European Society of Cardiology Heart Failure 5(6): 989-1007; Gorter, T. M., Obokata, M., Reddy, Y. N. V., Melenovsky, V., and Borlaug, B. A. 2018. “Exercise Unmasks Distinct Pathophysiologie Features in Heart Failure with Preserved Ejection Fraction and Pulmonary Vascular Disease,” European Heart Journal 39(30): 2825-2835; Mohananey, D., Heidari-Bateni. G., Villablanca, P. A., Iturrizaga Murrieta, J. C., Vlismas, P., and 4 others 2018. “Heart Failure with Preserved Ejection Fraction—A Systematic Review and Analysis of Perioperative Outcomes,” Journal of Cardiothoracic and Vascular Anesthesia 32(5): 2423-2434; Zheng, S. L., Chan, F. T., Nabeebaccus, A. A., Shah, A. M., McDonagh, T., Okonko, D. O., and Ayis. S. 2018. “Drug Treatment Effects on Outcomes in Heart Failure with Preserved Ejection Fraction: A Systematic Review and Meta-analysis,” Heart (British Cardiac Society) 104(5): 407-415; Chauhan, C. 2017. “Living with Heart Failure with Preserved Ejection Fraction,” Journal of the American College of Cardiology. Heart Failure 5(3): 236-237; Guazzi, M., Dixon, D., Labate, V., Beussink-Nelson, L., Bandera, F., Cuttica, M. J., and Shah, S. J. 2017. “R[ight] V[entricular] Contractile Function and Its Coupling to Pulmonary Circulation in Heart Failure with Preserved Ejection Fraction: Stratification of Clinical Phenotypes and Outcomes,” Journal of the American College of Cardiology. Cardiovascular Imaging 10(10 Part B): 1211-1221; Melenovsky, V., Hwang, S., Lin, G., Redfield, M. M., and Borlaug. B. A. 2014. “Right Heart Dysfunction in Heart Failure with Preserved Ejection Fraction,” European Heart Journal 35(48): 3452-3462; Shah, A. M., Shah, S., Anand. I. S., Sweitzer, N. K., O'Meara, E., and 8 others 2014. “Cardiac Structure and Function in Heart failure with Preserved Ejection Fraction: Baseline Findings from the Echocardiographic Study of the Treatment of Preserved Cardiac Function Heart Failure with an Aldosterone Antagonist trial,” Circulation. Heart Failure 7(1): 104-115).

The transplantability of hearts with preserved or mid-range ejection fraction will increase as more effective treatments become available. Able to release agents into the general circulation and/or targeted to the site or sites of disease automatically, apply warmth, deliver electrical, and prospectively, leadless pacemaker or direct optogenetic or electrostimulation at nidi, the implant disorder response system will facilitate the application of new therapy both mid- and postprocedurally. “ . . . neprilysin inhibitor-angiotensin receptor blocker-combination (LCZ 696), ranolazine, or ivabradine . . . ” for example, has shown promise (Edelmann, F. 2015. “Facts and Numbers on Epidemiology and Pharmacological Treatment of Heart Failure with Preserved Ejection Fraction,” European Society of Cardiology Heart Failure 2(2): 41-45).

Restorative therapy for hearts affected thus is an area of active research. Hearts with mid-range ejection fraction are those most likely to prove remediable (Özlek. B., Özlek. E., Ağuş, H. Z., Tekinalp. M., Kahraman, S., and 15 others 2019. “Patients with HFpEF and HFmrEF Have Different Clinical Characteristics in Turkey: A Multicenter Observational Study,” European Journal of Internal Medicine 61:88-95; Özlek. B., Özlek, E., çelik, O., çil. C., Doğan, V., and 17 others 2018. “Rationale, Design, and Methodology of the APOLLON trial: A comprehensive, ObservationaL registry of heart failure with midrange and preserved ejectiON fraction,” Anatolian Journal of Cardiology (Istanbul, Turkey) 19(5): 311-318; Ozlek, B., Ozlek, E., Kahraman, S., Tekinalp, M., Zencirkiran Agus, H., and 15 others 2019. “Gender Disparities in Heart Failure with Mid-range and Preserved Ejection Fraction: Results from APOLLON [A comprehensive, ObservationaL registry of heart fai Lure with mid-range and preserved ejection fractiON] Study,” Anatolian Journal of Cardiology (Istanbul, Turkey) 21(5): 242-252; Ozlek, B., Özlek, E., Tekinalp. M., Kahraman, S., Ağuş, H. Z., and 15 others 2019. “Clinical Features of Heart Failure with Mid-range and Preserved Ejection Fraction in Octogenarians: Results of a Multicentre, Observational Study,” International Journal of Clinical Practice 73(4): e13341; Ozlek, B., Ozlek, E., Zencirkιran Agus, H., Tekinalp. M., Kahraman, S., and 15 others 2019. “Geographical Variations in Patients with Heart Failure and Preserved Ejection Fraction: A Sub-group Analysis of the APOLLON Registry,” Balkan Medical Journal (Istanbul, Turkey) 36(4): 235-244; Winchester, D. E. 2019. “Strategies for Management of Heart failure with Preserved Ejection Fraction,” Online, International Journal of Cardiology. Heart and Vasculature (Shannon, Ireland) 23: 100379; Webb, J., Draper, J., Fovargue, L., Sieniewicz, B., Gould, J., and 10 others 2018. “Is Heart Failure with Mid Range Ejection Fraction (HFmrEF) a Distinct Clinical Entity or an Overlap Group?,” Online, International Journal of Cardiology. Heart and Vasculature (Shannon, Ireland) 21:1-6)(see also, for example. Mesquita, E. T., Barbetta, L. M. D. S., and Correia, E. T. O. 2019. “Heart Failure with Mid-Range Ejection Fraction—State of the Art,” Arquivos Brasileiros de Cardiologia (Brazilian Archives of Cardiology, Sao Paulo, Brazil) 112(6): 784-790; Ilieιiu, A.M1, and Hodorogea, A. S. 2018. “Treatment of Heart Failure with Preserved Ejection Fraction,” Advances in Experimental Medicine and Biology 1067:67-87; Lund, L. H., Claggett. B., Liu, J., Lam, C. S., Jhund, P. S., and 7 others 2018. “Heart Failure with Mid-range Ejection Fraction in CHARM [candesartan in heart failure assessment of reduction in mortality and morbidity]: Characteristics, Outcomes, and Effect of Candesartan across the Entire Ejection Fraction Spectrum,” European Journal of Heart Failure 20(8): 1230-1239; Rickenbacher, P. 2018. “Heart Failure with “Mid-range” Ejection Fraction: A New Clinical Entity?,” (in German with abstract in English at Pubmed), Therapeutische Umschau. Revue Thérapeutique [Therapy Review] (Berne, Switzerland) 75(3): 170-173; Tschope, C., Birner, C., Böhm, M., Bruder, O., Frantz, S., and 5 others 2018. “Heart Failure with Preserved Ejection Fraction: Current Management and Future Strategies: Expert Opinion on the Behalf of the Nucleus of the “Heart Failure Working Group” of the German Society of Cardiology (DKG),” Clinical Research in Cardiology 107(1): 1-19; Chioncel. O., Lainscak, M., Seferovic, P. M., Anker, S. D., Crespo-Leiro, M. G., and 13 others 2017. “Epidemiology and One-year Outcomes in Patients with Chronic Heart Failure and Preserved, Mid-range and Reduced Ejection Fraction: An Analysis of the ESC [European Society of Cardiology] Heart Failure Long-term Registry,” European Journal of Heart Failure 19(12): 1574-1585; Farmakis, D., Simitsis, P., Bistola, V., Triposkiadis, F., Ikonomidis, I., and 6 others 2017. “Acute Heart Failure with Mid-range Left Ventricular Ejection Fraction: Clinical Profile, in-Hospital Management, and Short-term Outcome,” Clinical Research in Cardiology 106(5): 359-368; Katsi, V., Georgiopoulos, G., Laina, A., Koutli, E., Parissis, J., and 3 others 2017. “Left Ventricular Ejection Fraction as Therapeutic Target: Is It the Ideal Marker?,” Heart Failure Reviews 22(6): 641-655; Nauta, J. F., Hummel, Y. M., van Melle, J. P., van der Meer, P., Lam, C. S. P., Ponikowski, P., and Voors. A. A. 2017. “What Have We Learned about Heart Failure with Mid-range Ejection Fraction One Year after Its Introduction?,” European Journal of Heart Failure 19(12): 1569-1573; Jorde, U. P. and Fox, M. T. 2003. “The CHARM [candesartan in heart failure assessment of reduction in mortality and morbidity] Programme,” Lancet 362(9396): 1677-1679).

Other areas where direct electrical or optogenetic stimulation can be used to assist motile function while avoiding the shortcomings of mechanical devices are in deceased prospective organ donors on life support and as implants in patients. The electrical discharge and light emitting sources are best secured in place by nonjacketing side-entry connectors as described in U.S. Pat. No. 11,013,858, which can be made with hollow injectable anchoring needles able to deliver fluid agents, to work with the stimulatory energy. Devices such as heart-lung and extracorporeal membrane oxygenation machines are confined to the clinic and limited in duration of use.

Such means do not apply to critically weakened hearts, that is, in hearts lacking the strength to generate an adequate ejection fraction without added power, inotropes, a second heart to divide the load, and/or direct stimulation; however, end-stage impaired hearts requiring placement of an assist device are unacceptable for transplantation in any event. Hearts suitable for transplantation with the support of an implanted automatic disorder response system should retain sufficient strength to function with no more than the aid of pharmaceutical, and/or electrical, and prospectively, optogenetic stimulation.

Electrical or optogenetic stimulation should, however, allow hearts with sufficient viable but dysfunctional left ventricular muscle but rather neurological impairment, for example, important in ascertaining the restorative consequences of coronary artery bypass grafting, angioplasty, or the implantation of a ventricular assist device (see, for example, Toepfer, C. N., Sikkel, M. B., Caorsi, V., Vydyanath, A., Torre, I., and 7 others 2016. “A Post-MI Power Struggle: Adaptations in Cardiac Power Occur at the Sarcomere Level alongside MyBP-C [myosin binding protein-C] and RLC [myosin regulatory light chain] Phosphorylation,” American Journal of Physiology. Heart and Circulatory Physiology 311(2): H465-H475; Bayeva, M., Sawicki, K. T., Butler, J., Gheorghiade, M., and Ardehali, H. 2014. “Molecular and Cellular Basis of Viable Dysfunctional Myocardium,” Circulation. Heart Failure 7(4): 680-691; Meluzin, J., Cerny, J., Groch, L., Frelich, M., Stetka, F., Spinarová, L., and Panovský, R. 2003. “Prognostic Importance of the Quantification of Myocardial Viability in Revascularized Patients with Coronary Artery Disease and Moderate-to-severe Left Ventricular Dysfunction,” International Journal of Cardiology 90(1): 23-32; Kalra, D. K. and Zoghbi. W. A. 2002. “Myocardial Hibernation in Coronary Artery Disease,” Current Atherosclerosis Reports 4(2): 149-155; Meluzin, J., Cerny, J., Frelich, M., Stetka, F., Spinarova, L., Popelova, J., and Stipal. R. 1998. “Prognostic Value of the Amount of Dysfunctional but Viable Myocardium in Revascularized Patients with Coronary Artery Disease and Left Ventricular Dysfunction—a Multicenter Study,” Journal of the American College of Cardiology 32(4): 912-920) and other prospective graft organs similarly affected to be salvaged (see, for example, Spencer, N. J., Hibberd. T., Feng. J., and Hu, H. 2019. “Optogenetic Control of the Enteric Nervous System and Gastrointestinal Transit,” Expert Review of Gastroenterology and Hepatology 13(4): 281-284). Using the methods delineated herein, a usable heart must not be so impaired as to necessitate a ventricular assist device. These are neither applied to the heart of a deceased prospective heart donor while remaining on life support for the harvesting of other organs nor applied during or after transfer to the recipient. Hearts this impaired should be rejected.

When the need is dire, hearts which have recovered with the aid of an assist device on a short-term basis should be considered (see, for example, Pham, B. N. and Chaparro, S. V. 2019. “Left Ventricular Assist Device Recovery: Does Duration of Mechanical Support Matter?,” Heart Failure Reviews 24(2): 237-244; Castillero, E., Ali, Z. A., Akashi, H., Giangreco, N., Wang, C., and 19 others 2018. “Structural and Functional Cardiac Profile after Prolonged Duration of Mechanical Unloading: Potential Implications for Myocardial Recovery,” American Journal of Physiology. Heart and Circulatory Physiology 315(5): H1463-H1476; Persoon, S., Paulus, M., Hirt. S., Jungbauer, C., Dietl. A., and 4 others 2018. “Cardiac Unloading by LVAD Support Differentially Influences Components of the cGMP-PKG Signaling Pathway in Ischemic and Dilated Cardiomyopathy,” Heart and Vessels (Tokyo, Japan) 33(8): 948-957; Ito. E., Miyagawa, S., Fukushima, S., Yoshikawa, Y., Saito, S., and 8 others 2017. “Histone Modification is Correlated with Reverse Left Ventricular Remodeling in Nonischemic Dilated Cardiomyopathy,” 104(5): 1531-1539; Miyagawa, S., Toda, K., Nakamura. T., Yoshikawa, Y., Fukushima, S., and 4 others 2016. “Building a Bridge to Recovery: The Pathophysiology of LVAD-induced Reverse Modeling in Heart Failure,” Surgery Today 46(2): 149-154; Phan, K., Huo, Y. R., Zhao, D. F., Yan, T. D., and Tchantchaleishvili, V. 2016. “Ventricular Recovery and Pump Explantation in Patients Supported by Left Ventricular Assist Devices: A Systematic Review,” American Society for Artificial Internal Organs Journal 62(3): 219-231; Heerdt, P. M., Klotz, S., and Burkhoff, D. 2006. “Cardiomyopathie Etiology and SERCA2a Reverse Remodeling during Mechanical Support of the Failing Human Heart,” Anesthesia and Analgesia 102(1): 32-37).

Ventricular assist devices are further addressed in section 2b(1)(b)(b5), entitled Heterotopic Double Heart Metered Compound Vascular Bypass, or Switched-organ Transplantation [and so on]. These are effective for bridging to transplantation, for example, but often produce adverse sequelae in proportion to the duration of use. With the relief in load, some patients do experience reversal of the remodeling that had taken place to compensate for the weakened left ventricle; however, such cases are exceptional. Double heart metered compound vascular bypass, or sudden switched-organ transplantation is intended for use in prospective heart recipients, not donors. For weaker native hearts, the second heart can be thought of as a form of assist device. Severely impaired hearts in prospective recipients are well served by an assist device. For reasons of surgical trauma, marginal hearts in deceased donors would best be treated by direct stimulation and drugs.

Use along the urinary and digestive tracts of commercially available neuromodulators is of long standing. While application to patients with heart failure and recipients of marginal hearts are contemplated, the main objective here is to sustain or assist motile function in a deceased organ donor with a marginal heart that would otherwise be rejected for transplantation. For these purposes, a means for assisting ventricular function without the surgical trauma associated with the implantation of an assist device would both allow the use, and less impose additional impairment upon, less competent hearts.

Even miniaturized, assist devices are larger than and no more amenable to endoscopic and robotic implantation than are stimulatory probes (see, for example, Carrozzini, M., Bejko, J., Guariento, A., Rubino, M., Bianco, R., and 4 others 2019. “Minimally Invasive Implantation of Continuous Flow Left Ventricular Assist Devices: The Evolution of Surgical Techniques in a Single-Center Experience,” Artificial Organs 43(3): E41-E52; Reichart. D., Brand, C. F., Bernhardt, A. M., Schmidt, S., Schaefer, A., and 5 others 2019. “Analysis of Minimally Invasive Left Thoracotomy HVAD [registered trademark, Medtronics Corporation, Framingham, Massachusetts] Implantation—A Single-center Experience,” Thoracic and Cardiovascular Surgeon (Stuttgart, Germany) 67(3): 170-175; Wachter, K., Franke, U. F. W., Rustenbach, C. J., and Baumbach, H. 2019. “Minimally Invasive versus Conventional LVAD-implantation—An Analysis of the Literature,” Thoracic and Cardiovascular Surgeon (Stuttgart, Germany) 67(3): 156-163; Deniz, E., Chatterjee, A., Feldmann, C., Hanke, J. S., Dogan, G., and 4 others 2018. “How To Do It: Tips and Tricks of Minimal- invasive HVAD® Implantation—the Lateral approach,” Journal of Thoracic Disease 10 (Supplement 15): S1829-S1833; Mohite. P. N., Sabashnikov, A., Raj. B., Hards. R., Edwards, G., and 10 others 2018. “Minimally Invasive Left Ventricular Assist Device Implantation: A Comparative Study,” Artificial Organs 42(12): 1125-1131; Ricklefs. M., Hanke, J. S., Dogan, G., Napp. L. C., Feldmann, C., Haverich, A., and Schmitto, J. D. 2018. “Less Invasive Surgical Approaches for Left Ventricular Assist Device Implantation,” Seminars in Thoracic and Cardiovascular Surgery 30(1): 1-6; Hanke, J. S., Rojas, S. V., Avsar, M., Haverich, A., and Schmitto. J. D. 2015. “Minimally-invasive LVAD Implantation: State of the Art,” Current Cardiology Reviews 11(3): 246-251; Rojas. S. V., Avsar, M., Hanke, J. S., Khalpey, Z., Maltais, S., Haverich, A., and Schmitto, J. D. 2015. “Minimally Invasive Ventricular Assist Device Surgery,” Artificial Organs 39(6): 473-479).

Involving greater complexity, mechanical assist devices include more parts with different distribution and are more susceptible to complications with increased probability over time. The surgical procedures to place these and total artificial hearts poses numerous serious risks, and when successful, the patient is strapped with the need to wear outside components such as a power pack and controller which must be worn at all times.

This to support a complex device with many parts any of which might fail at any moment with grave consequences (see, for example, Asleh, R., Alnsasra, H., Daly, R. C., Schettle, S. D., Briasoulis, A., and 9 others 2019. “Predictors and Clinical Outcomes of Vasoplegia in Patients Bridged to Heart Transplantation with Continuous-flow Left Ventricular Assist Devices,” Journal of the American Heart Association (Oxford, England) 8(22): e013108; Boruah, P., Saqib, N., Barooah, J., Baruah, D., and Sharma, P. 2019. “Left Ventricular Assist Device: What the Internist Needs to Know. A Review of the Literature,” Online, Curēus (Palo Alto, California) 11(4): e4399; Cho, S. M., Moazami, N., Katz, S., Bhimraj, A., Shrestha, N. K., and Frontera, J. A. 2019. “Stroke Risk following Infection in Patients with Continuous-flow Left Ventricular Assist Device,” Neurocritical Care 31(1): 72-80; Gulati, G., Grandin, E. W., Kennedy, K., Cabezas, F., DeNofrio, D. D., and 7 others 2019. “Preimplant Phosphodiesterase-5 Inhibitor Use Is Associated with Higher Rates of Severe Early Right Heart Failure after Left Ventricular Assist Device Implantation,” Circulation. Heart Failure 12(6): e005537; Gulati, G., Kiernan, M., and Sabe, M. A. 2019. “Massive Retroperitoneal Hemorrhage after External Shock Wave Lithotripsy in a Patient with a Left Ventricular Assist Device,” Urology Case Reports 28:101033; Harper, R., Ludwig, J., Morcos, M., and Morris, S. 2019. “Myocardial Irritation from a Left Ventricular Assist Device Resulting in Refractory Ventricular Tachycardia,” Journal of Emergency Medicine 56(1): 87-93; Hodson, T., West. J. M., Poteet, S., Lee, P. H., and Valerio, I. L. 2019. “Instillation Negative Pressure Wound Therapy: A Role for Infected LVAD Salvage,” Advances in Wound Care (New Rochelle, New York) 8(3): 118-124; Li, X., Kondray. V., Tavri. S., Ruhparwar, A., Azeze, S., and 3 others 2019. “Role of Imaging in Diagnosis and Management of Left Ventricular Assist Device Complications,” International Journal of Cardiovascular Imaging 35(7): 1365-1377; Long, B., Robertson, J., Koyfman, A., and Brady, W. 2019. “Left Ventricular Assist Devices and Their Complications: A Review for Emergency Clinicians,” American Journal of Emergency Medicine 37(8): 1562-1570; Schmack, B., Sabashnikov, A., Weymann, A., Zeriouh, M., Koch, A., and 3 others 2019. “Left Ventricular Assist Devices Exchange: Why. When, and How to Do It-Experience from Experts,” Journal of Thoracic Disease 11 (Supplement 6): S963-S966; Sen, A., Larson, J. S., Kashani, K. B., Libricz, S. L., Patel, B. M., and 4 others 2016. “Mechanical Circulatory Assist Devices: A Primer for Critical Care and Emergency Physicians,” Critical Care (London, England) 20(1): 153; Thiha, S., Zaidi, A. R. Z., Robert, C. A., Abbas, M. K., and Malik, B. H. 2019. “A Rising Hope of an Artificial Heart: Left Ventricular Assisted Device-Outcome, Convenience, and Quality of Life,” Online, Cureus (Palo Alto, California) 11(9): e5617; Turner, K. R. 2019. “Right Ventricular Failure after Left Ventricular Assist Device Placement—The Beginning of the End of Just Another Challenge?,” Journal of Cardiothoracic and Vascular Anesthesia 33(4): 1105-1121; Wrobel, C. A., Drazner, M. H., Ayers. C. R., Pham, D. D., La Hoz, R. M., and 8 others 2019. “Delayed Febrile Response with Bloodstream Infections in Patients with Continuous-flow Left Ventricular Assist Devices,” Journal of Investigative Medicine 67(3): 653-658; Aissaoui, N., Jouan, J., Gourjault, M., Diebold, B., Ortuno, S., and 4 others 2018. “Understanding Left Ventricular Assist Devices,” Blood Purification 46(4): 292-300; Brady, W., Weigand, S., and Bergin, J. 2018. “Ventricular Assist Device in the Emergency Department: Evaluation and Management Considerations,” American Journal of Emergency Medicine 36(7): 1295-1299; de Waal, E. E. C., van Zaane, B., van der Schoot, M. M., Huisman, A., Ramjankhan, F., van Klei, W. A., and Marczin, N. 2018. “Vasoplegia after Implantation of a Continuous Flow Left Ventricular Assist Device: Incidence, Outcomes and Predictors,” Online, BioMed Central Anesthesiology 18(1): 185; Izzy. S., Rubin, D. B., Ahmed, F. S., Akbik, F., Renault, S., and 5 others 2018. “Cerebrovascular Accidents During Mechanical Circulatory Support: New Predictors of Ischemic and Hemorrhagic Strokes and Outcome,” Stroke 49(5): 1197-1203; Kemaloğlu, C., Altekin, R. E., and Bayezid, O. 2018. “First Successful Percutaneous Treatment of a Totally Occluded HeartWare Outflow Graft: Case Report and Literature Review,” Anatolian Journal of Cardiology 19(5): 341-345; Mckillip, R. P., Gopalsami, A., Montoya, M., Kim, G., Walter, J. J., Juricek, C., and Shappell, E. 2018. “Analysis of Patients with Ventricular Assist Devices Presenting to an Urban Emergency Department,” Western Journal of Emergency Medicine 19(6): 907-911; Miklin, D., Lewis, I., and Lieberman, H. 2018. “Bowel Obstruction Due to Retained Intraperitoneal Left Ventricular Assist Device (LVAD) Driveline,” Journal of Cardiothoracic Surgery 13(1): 46: Najjar, E., Hallberg Kristensen, A., Thorvaldsen, T., Hubbert, L., Svenarud, P., and 3 others 2018. “Controller and Battery Changes Due to Technical Problems Related to the HVAD® Left Ventricular Assist Device—A Single Center Experience,” Journal of Cardiothoracic Surgery 13(1): 74; Prichard, R., Kershaw. L., Goodall. S., Davidson, P., Newton, P., and 3 others 2018. “Left Ventricular Device Implantation Impacts on Hospitalisation Rates, Length of Stay, and Out of Hospital Time,” Heart, Lung, and Circulation 27(6): 708-715; Rice, C. J., Cho, S. M., Zhang, L. Q., Hassett, C., Starling, R. C., and Uchino, K. 2018. “The Management of Acute Ischemic Strokes and the Prevalence of Large Vessel Occlusion in Left Ventricular Assist Device,” Cerebrovascular Diseases 46(5-6): 213-217; Ryu, B., Ishikawa. T., Yamaguchi, K., Matsuoka, G., Eguchi, S., and Kawamata, T. 2018. “Long-term Outcomes following Thrombectomy for Acute Ischemic Stroke in Patients with a Left Ventricular Assist Device: A Case Series and Literature Review,” Acta Neurochirurgica (Vienna, Austria) 160(9): 1729-1735; Soliman, O. I. I., Akin, S., Muslem, R., Boersma, E., Manintveld, O. C., and 7 others 2018. “Derivation and Validation of a Novel Right-sided Heart Failure Model after Implantation of Continuous Flow Left Ventricular Assist Devices: The EUROMACS (European Registry for Patients with Mechanical Circulatory Support) Right-sided Heart Failure Risk Score,” Circulation 137(9): 891-906; Tainter, C. R., Braun, O. O., Teran, F., Nguyen, A. P., Robbins, K., and 9 others 2018. “Emergency Department Visits among Patients with Left Ventricular Assist Devices,” Internal and Emergency Medicine 13(6): 907-913; Trinquero. P., Pirotte, A., Gallagher, L. P., Iwaki, K. M., Beach, C., and Wilcox, J. E. 2018. “Left Ventricular Assist Device Management in the Emergency Department,” Western Journal of Emergency Medicine 19(5): 834-841; Cho, S. M., Moazami, N., and Frontera, J. A. 2017. “Stroke and Intracranial Hemorrhage in HeartMate II and HeartWare Left Ventricular Assist Devices: A Systematic Review,” Neurocritical Care 27(1): 17-25; Dang, G., Epperla, N., Muppidi. V., Sahr, N., Pan, A., Simpson, P., and Baumann Kreuziger, L. 2017. “Medical Management of Pump-related Thrombosis in Patients with Continuous-flow Left Ventricular Assist Devices: A Systematic Review and Meta-Analysis,” American Society for Artificial Internal Organs Journal 63(4): 373-385; Frontera, J. A., Starling. R., Cho, S. M., Nowacki, A. S., Uchino, K., and 3 others 2017. “Risk Factors, Mortality, and Timing of Ischemic and Hemorrhagic Stroke with Left Ventricular Assist Devices,” Journal of Heart and Lung Transplantation (New York, New York) 36(6): 673-683; Kormos. R. L., McCall. M., Althouse, A., Lagazzi, L., Schaub. R., and 7 others 2017. “Left Ventricular Assist Device Malfunctions: It is More than Just the Pump,” Circulation 136(18): 1714-1725; Pendyal. A., Chien, C. V., Mudd, J. O., and Gelow, J. M. 2017. “Minimally Invasive LVAD Deactivation in a 65-Year-Old Man with Recurrent Pump Thrombosis and Left Ventricular Recovery,” Texas Heart Institute Journal 44(1): 70-72; Chua, K. C., Tan, T. E., Lim, C. H., Kerk, K. L., and Sivathasan, C. 2016. “Contained Rupture in HeartMate II Outflow Graft Bend Relief Disconnection,” Asian Cardiovascular and Thoracic Annals 24(3): 269-271; Fitzgibbon, J., Kman, N. E., and Gorgas, D. 2016. Asymptomatic Sustained Polymorphic Ventricular Tachycardia in a “Patient with a Left Ventricular Assist Device: Case Report and What the Emergency Physician Should Know,” Journal of Emergency Medicine 50(3): e135-e141; Health Quality Ontario 2016. “Left Ventricular Assist Devices for Destination Therapy: A Health Technology Assessment,” Ontario Health Technology Assessment Series 16(3): 1-60. Online at https://www.ncbi.nlm.nih.gov/pmc/articles/PMC4761917/; Koksel. U., Erbasan, O., Bayezid, O., Kemaloğlu, C., Özçobanoğlu, S., Gölbaşι, I., and Türkay, C. 2016. “Thrombosis in Continuous Flow Left Ventricular Assist Devices: Our Clinical Experience with Medical and Surgical Management,” Transplantation Proceedings 48(6): 2162-2167; Robertson, J., Long. B., and Koyfman, A. 2016. “The Emergency Management of Ventricular Assist Devices,” American Journal of Emergency Medicine 34(7): 1294-1301; Suarez, J. and Yang, D. 2016. “Gastrointestinal Symptoms from Left Ventricular Assist Device External Compression of the Gastric Lumen,” American College of Gastroenterology Case Reports Journal 3(4): e180; Tuncer, O. N., Kemaloğlu, C., Erbasan, O., Gölbaşι, I., Türkay, C., and Bayezid, O 2016. “Outcomes and Readmissions after Continuous Flow Left Ventricular Assist Device: Heartmate II versus Heartware Ventricular Assist Device,” Transplantation Proceedings 48(6): 2157-2161; Kilic, A., Acker, M. A., and Atluri, P. 2015. “Dealing with Surgical Left Ventricular Assist Device Complications,” Journal of Thoracic Disease 7(12): 2158-2164).

The shortcomings of these devices in terms of hindrance, risk upon implantation, power consumption, and risk of mechanical failure compared to a compound vascular bypass heterotopic double heart transplant are addressed below in section 2b(1)(b)(b5), entitled Heterotopic Double Heart Metered Compound Vascular Bypass, or Switched-organ Transplantation [and so on] An implanted disorder response system can support, or in a double heart transplant, administer the operation as well as replace such a device. This leaves the surgery of placement and the immunological challenges associated with any transplant as risks; however, the level of contingency the patient will face should be much reduced.

The double heart or heterotopic compound vascular transplant has significant potential in poor parts of the world where replacement hearts in recently deceased donors kept on life support past death can be provided. In 2017. “The mean cost of LVAD implantation was $175,420.00 . . . ” Along with outpatient follow-up costs and readmissions, the total came to three-quarters of a million dollars (Baras Shreibati, J., Goldhaber-Fiebert, J. D., Banerjee, D., Owens, D. K., and Hlatky, M. A. 2017. “Cost-effectiveness of Left Ventricular Assist Devices in Ambulatory Patients with Advanced Heart Failure,” Online, Journal of the American College of Cardiology. Heart Failure 5(2): 110-119).

Percutaneously inserted intraaortic balloon pumps and ventricular assist devices impose a considerable demand for power with the need to wear a power pack, include numerous mechanical parts, and are often placed for short term use. In contrast, the power requirement to sustain the mechanical support of a double heart transplant—when even necessary—is much less, requires a much smaller and lighter power pack, and the vascular servovalves used are mechanically simple and directly targetable for the release of an anticoagulant or antimicrobial as the need arises. Where double heart transplantation is intended as destination therapy with removal should the native heart recover possible, mechanical devices are usually not meant to remain permanently as destination therapy but rather bridge to transplantation pending availability of a replacement heart, or to allow time during which the patient can be stabilized for transplantation, or to allow recovery with device removal.

Significantly, the contraindications to assist device placement—right ventricular dysfunction; acute cardiogenic shock with a neurological compromise; coexisting severe terminal comorbidity: active bleeding or thrombocytopenia; hypertrophic cardiomyopathy or a large ventricular septal defect; body surface area less than 1-½ meters; and a patient who cannot be depended upon for prescription and maintenance compliance (Vaidya, Y., Riaz, S. and Dhamoon, A. S. 2020. “Left Ventricular Assist Devices (LVAD),” Online, Treasure Island, Florida: StatPearls Publishing Co., at https://www.ncbi.nlm.nih.gov/books/NBK499841/) do not apply to double heart transplantation. Except that a relative or attendant must assure that drug reservoirs are replenished, the implanted automatic disorder response system essentially eliminates the requirement for compliance.

As with a mechanical device, recovery of the native heart following a double heart transplant can allow the implant to be removed (see, for example, Drakos, S. G., Pagani, F. D., Lundberg, M. S., and Baldwin, J. T. 2017. “Advancing the Science of Myocardial Recovery with Mechanical Circulatory Support: A Working Group of the National, Heart, Lung, and Blood Institute,” Journal of the American College of Cardiology. Basic to Translational Science 2(3): 335-340; Dandel, M. and Hetzer, R. 2015. “Myocardial Recovery during Mechanical Circulatory Support: Weaning and Explantation Criteria,” Heart, Lung, and Vessels 7(4): 280-288; Selzman, C. H., Madden, J. L., Healy, A. H., Mckellar, S. H., Koliopoulou, A., Stehlik, J., and Drakos, S. G. 2015. “Bridge to Removal: A Paradigm Shift for Left Ventricular Assist Device Therapy,” Annals of Thoracic Surgery 99(1): 360-367; Lamarche, Y., Kearns, M., Josan, K., Bashir, J., Ignaszewski, A., and 4 others 2011. “Successful Weaning and Explantation of the Heartmate II Left Ventricular Assist Device,” Canadian Journal of Cardiology 27(3): 358-362).

Disorders of coagulation a frequent complication with ventricular assist devices, the ability of the implanted disorder response system to directly target drugs whether coagulants (hemostats) or anticoagulants to the device- or to any other target—with means for preventing continued travel through the circulatory system should avert bleeding of the upper gastrointestinal tract as a common complication (Hilal. T., Mudd. J., and DeLoughery, T. G. 2019. “Hemostatic Complications Associated with Ventricular Assist Devices,” Research and Practice in Thrombosis and Haemostasis 3(4): 589-598; Rauch, A., Susen, S., and Zieger, B. 2019. “Acquired von Willebrand Syndrome in Patients with Ventricular Assist Device,” Online, Frontiers in Medicine (Lausanne, Switzerland) 6:7; Imamura, T., Chung, B., Nguyen, A., Sayer, G., and Uriel, N. 2018. “Clinical Implications of Hemodynamic Assessment during Left Ventricular Assist Device Therapy,” Journal of Cardiology 71(4): 352-358; Kim, J. H., Brophy, D. F., and Shah, K. B. 2018. “Continuous-flow Left Ventricular Assist Device-related Gastrointestinal Bleeding,” Cardiology Clinics 36(4): 519-529; Molina, T. L., Krisl, J. C., Donahue, K. R., and Varnado, S. 2018. “Gastrointestinal Bleeding in Left Ventricular Assist Device: Octreotide and Other Treatment Modalities,” American Society for Artificial Internal Organs Journal 64(4): 433-439; Mondal, N. K., Chen, Z., Trivedi, J. R., Sorensen, E. N., Pham, S. M., and 3 others 2018. “Association of Oxidative Stress and Platelet Receptor Glycoprotein GPIbα and GPVI Shedding during Nonsurgical Bleeding in Heart Failure Patients with Continuous-flow Left Ventricular Assist Device Support,” American Society of Artificial Internal Organs Journal 64(4): 462-471; Muslem, R., Caliskan, K., and Leebeek, F. W. G. 2018. “Acquired Coagulopathy in Patients with Left Ventricular Assist Devices,” Journal of Thrombosis and Haemostasis 16(3): 429-440; Welden, C. V., Truss, W., McGwin, G., Weber, F., and Peter, S. 2018. “Clinical Predictors for Repeat Hospitalizations in Left Ventricular Assist Device (LVAD) Patients with Gastrointestinal Bleeding,” Gastroenterology Research 11(2): 100-105; Li, F., Hinton, A., Chen, A., Mehta, N. K., Eldika. S., and 4 others 2017. “Left Ventricular Assist Devices Impact Hospital Resource Utilization without Affecting Patient Mortality in Gastrointestinal Bleeding,” Digestive Diseases and Sciences 62(1): 150-160; Mondal, N. K., Chen, Z., Trivedi, J. R., Sorensen, E. N., Pham, S. M., and 3 others 2017. “Oxidative Stress Induced Modulation of Platelet Integrin α2bβ3 Expression and Shedding May Predict the Risk of Major Bleeding in Heart Failure Patients Supported by Continuous Flow Left Ventricular Assist Devices,” Thrombosis Research 158:140-148; Jabbar, H. R., Abbas, A., Ahmed, M., Klodell, C. T. Jr., Chang, M., Dai, Y., and Draganov, P. V. 2015. “The Incidence, Predictors and Outcomes of Gastrointestinal Bleeding in Patients with Left Ventricular Assist Device (LVAD),” Digestive Diseases and Sciences 60(12): 3697-3706).

However, coagulopathy can occur in cardiovascular disease with no involvement of a continuous flow ventricular assist device. “Various cardiovascular diseases, such as aortic stenosis, hypertrophic obstructive cardiomyopathy (HOCM), and several congenital structural diseases, as well as mechanical circulatory support systems, generate excessive high shear stress in the bloodstream. These cause excessive cleavage of VWF [von Willebrand factor] multimers resulting in a loss of HMW [high molecular weight] multimers, known as acquired von Willebrand syndrome . . . ” (Horiuchi, H., Doman, T., Kokame, K., Saiki, Y., and Matsumoto, M. 2019. “Acquired von Willebrand Syndrome Associated with Cardiovascular Diseases,” Journal of Atherosclerosis and Thrombosis 26(4): 303-314)(see also, for example, Mehta, R., Athar, M., Girgis, S., Hassan, A., and Becker, R. C. 2019. “Acquired von Willebrand Syndrome (AVWS) in Cardiovascular Disease: A State of the Art Review for Clinicians,” Journal of Thrombosis and Thrombolysis 48(1): 14-26).

Regardless of the cause or causes, adverse sequelae may necessitate device replacement, or exchange, and as does any surgical procedure, this too poses the risk of complications. “Device exchange is associated with significant risk, including hemorrhage, stroke, air embolism, and death” (Bhama, J. K. and Bansal. A. 2018. “Left Ventricular Assist Device Inflow Cannula Position May Contribute to the Development of HeartMate II Left Ventricular Assist Device Pump Thrombosis,” Ochsner Journal 18(2): 131-135). The contrary view has also been expressed (Agarwal. R., Kyvemitakis, A., Soleimani, B., Milano, C. A., Davis, R. P., and 5 others 2019. “Clinical Experience of HeartMate II to HeartWare Left Ventricular Assist Device Exchange: A Multicenter Experience,” Annals of Thoracic Surgery 108(4): 1178-1182; Exchange for upgrade to an improved device, however, usually when the original requires removal in any event, appears to present an acceptable risk to benefit ratio (see, for example, Chou, B. P., Lamba, H. K., Cheerna, F. H., Civitello, A. B., Delgado, R. M., and 7 others 2020. “Outcomes of Repeat Left Ventricular Assist Device Exchange,” American Society for Artificial Internal Organs Journal 66(1): 64-68; Imamura. T., Narang, N., Rodgers. D., Nguyen. A., Ota. T., and 6 others 2020. “Outcomes following Left Ventricular Assist Device Exchange,” Journal of Cardiac Surgery January 16; Urban, M., Um, J., Moulton, M., Stoller, D., Zolty, R., and 4 others 2020. “Recurrent Pump Thrombosis is Common after Axial Continuous-flow Left Ventricular Assist Device Exchange,” International Journal of Artificial Organs 43(2): 109-118; Vaidya, Y., Riaz, S. and Dhamoon, A. S. 2020. Op cit. this section; Bauer, T. M., Choi, J. H., Luc. J. G. Y., Weber, M. P., Moncho Escriva, E., and 8 others 2019. “Device Exchange versus Nonexchange Modalities in Left Ventricular Assist Device-specific Infections: A Systematic Review and Meta-analysis,” Artificial Organs 43(5): 448-457; Beaupre, R. A., Alnajar, A., Sugiura, T., Chou, B., Lamba, H. K., and 5 others 2019. “Device Exchange from Heartmate II to HeartWare HVAD,” Journal of Cardiac Surgery 34(11): 1204-1207; Takeda, K., Takayama, H., Sanchez, J., Cevasco, M., Yuzefpolskaya, M., Colombo, P. C., and Naka, Y. 2019. “Device Exchange from HeartMate II to HeartMate 3 Left Ventricular Assist Device,” Interactive Cardiovascular and Thoracic Surgery 29(3): 430-433; Hanke, J. S., Dogan, G., Wert, L., Ricklefs. M., Heimeshoff. J., and 4 others 2018. “Left Ventricular Assist Device Exchange for the Treatment of HeartMate II Pump Thrombosis,” Journal of Thoracic Disease 10 (Supplement 15): S1728-S1736; Trinquero. P., Pirotte, A., Gallagher, L. P., Iwaki, K. M., Beach, C., and Wilcox, J. E. 2018, Op cit., this section; Hanke, J. S., Rojas, S. V., Dogan, G., Feldmann, C., Beckmann, E., and 9 others 2017. “First Series of Left Ventricular Assist Device Exchanges to HeartMate 3,” European Journal of Cardiothoracic Surgery 51(5): 887-892; Shaikh, A. F., Joseph, S. M., Lima, B., Hall, S. A., Malyala, R., and 3 others 2017. “HeartMate II Left Ventricular Assist Device Pump Exchange: A Single-institution Experience,” Thoracic and Cardiovascular Surgeon 65(5): 410-414; Tsubota, H., Ribeiro, R. V. P., Billia. F., Cusimano, R. J., Yau, T. M., and 3 others 2017. “Left Ventricular Assist Device Exchange: The Toronto General Hospital Experience,” Canadian Journal of Surgery 60(4): 253-259; Berg. K. B. and Klodell, C. T. 2014. “Outflow Graft Thrombectomy during HeartMate II Left Ventricular Assist Device Exchange,” Interactive Cardiovascular and Thoracic Surgery 19(4): 714- 715: Stulak, J. M., Cowger, J., Haft, J. W., Romano, M. A., Aaronson, K. D., and Pagani, F. D. 2013. “Device Exchange after Primary Left Ventricular Assist Device Implantation: Indications and Outcomes,” Annals of Thoracic Surgery 95(4): 1262-1268; additional reference provided in section 2b(1)(b)(5)).

The intensive and immediate support of a fully implanted automatic ambulatory automatic disorder response system as described in U.S. Pat. No. 11,759,186, entitled Ductus Side-entry Jackets and Prosthetic Disorder Response Systems, and double heart metered switch heart transplantation as described below in section 2b(1)(b)(b5), entitled Heterotopic Double Heart Metered Compound Vascular Bypass, or Switched-organ Transplantation [and so on], have as a primary object the provision of alternatives to conventional treatment and liberalization in the criteria governing, and therewith, expansion in the pool of usable hearts. Harvesting graft organs upon death of the donor having remained on life support without the loss of perfusion and continuous transfer into the circulatory system of the recipient also protect graft organs against terminal damage that could disqualify these for transplantation, thus also increasing the number of suitable organs.

If placement of a ventricular assist device is likely to succeed, then that course is to be preferred to the double heart transplant; however, when reverse remodeling indicative of myocardial recovery justifies explantation, removal likewise poses the risk of trauma and complications, such as due to the nonremoval of all parts and/or mediastinal dissection and apical ventriculoplasty. A recently developed minimally invasive transcatheter technique, decommissioning, disables the implanted assist device and leaves it in place, materially reducing the risks of open removal—(Moroni, F., Shah, K. B., Quader, M. A., Klein, K., Smallfield, M. C., Parris, K. E., and Gertz, Z. M. 2022. “Percutaneous Approach to Left Ventricular Assist Device Decommissioning,” Online, Catheterization and Cardiovascular Interventions (New York, New York) 100(1): 169-174).

However, recurrent infection in 41% of cases remains a problem (Gerhard, E. F., Wang, L., Singh, R., Schueler, S., Genovese, L. D., and 9 others 2021. “LVAD Decommissioning for Myocardial Recovery: Long-term Ventricular Remodeling and Adverse Events,” Online, Journal of Heart and Lung Transplantation (New York, New York) 40(12): 1560-1570)(see also, for example, Smith, N. J., Collar, N., Duvvuri. P. Miles, B. Wu, R., and 4 others 2022. “Protocolized Screening Effectively Identifies Myocardial Recovery following Destination Therapy Left Ventricular Assist Device Implantation,” Artificial Organs 46(8): 1636-1648; Gallo, M., Trivedi, J. R., Monreal, G., Birks, E. J., and Slaughter, M. S. 2018. “Left Ventricular Assist Device Explantation after Myocardial Recovery,” Multimedia Manual of Cardiothoracic Surgery 2018; Saito, S., Toda, K., Miyagawa, S., Yoshikawa, Y., Hata. H., and 3 others 2018. “Colonic Fistula Caused by Remaining Inflow Cannula 14 Years after Left Ventricular Assist Device Explantation,” Journal of Artificial Organs 21(3): 356-358; Pendyal, A., Chien, C. V., Mudd, J. O., and Gelow, J. M. 2017, Op cit.; Sainte, S., Gewillig, M., Droogne, W., Van Cleemput, J., Vanhaecke, J., Meyns, B., and Rega, F. 2014. “Explantation of a CircuLite Left Ventricular Assist Device without Removal of the Inflow Cannula: How to do It?,” Interactive Cardiovascular and Thoracic Surgery 18(3): 393-395).

Following a double heart switch transplantation, both hearts are relieved from their previous loads and supported by the implanted automatic disorder response system. Because double heart transplantation allows adjustment in the combined or net ejection fraction, there is no concern that the one or two donor hearts working together would initially present absolute stroke volumes so high as to precipitate coma or convulsions as experienced in pediatric cases. Thus, impaired, either or both hearts by itself might not present normal ejective strength at first, but by sequencing every third or so systole of either in close succession-even without the use of inotropes—an ejection fraction falling within the normal range can be achieved.

Moreover, adjustment in the frequency of systole switching can be used to positively encourage recovery. Even without this capability, given time, such a new combination to include an additional heart would adapt to the changed physiological setting. Such a circumstance is presaged, for example, during the period following transplantation of an oversized heart. The overly large and powerful replacement heart generates a cardiac output that initially and for a number of days produces hypertension. However, this condition spontaneously resolves so that medical support is essential only during the adaptive period.

In the brain, this may cause reactive vasoconstriction that induces a convulsion or coma. However, this condition is dispelled within days during which the new heart adapts to the lesser load placed upon it (Shin, H. J., Jhang, W. K., Park, J. J., Yun, T. J., Kim, Y. H., and 3 others 2011. “Heart Transplantation in Pediatric Patients: Twelve-year Experience of the Asan Medical Center,” Journal of Korean Medical Science 26(5): 593-598). In the situation cited, the oversized heart adapts to the abrupt reduction in load while the patient is still in the hospital with clinicians to monitor and treat the condition. In a double heart switch transplant, the effective ejection fraction of both hearts working together in alternation is adjustable. This confers more than the selection of a second heart widely different in size; in fact, for placement in the chest as preferred, the choice of a smaller, healthier heart should be favored.

Adaptation consists first of relaxation-cessation in the expenditure of energy needlessly; however, if the reduction in load continues, the excess ventricular power will eventually decline. Since the load on either heart is controllable, it is prudent to maintain each heart at a level of strength somewhat in excess of that needed ordinarily. Under constant monitoring by implanted sensors, beat timing circuitry, and a control processor able to immediately target any fluid drug to either or both hearts, the likelihood of a cardiac or vascular accident is much reduced. Nevertheless, should either heart begin to malfunction, essentially to fibrillate, then having provided a safety margin will allow the unaffected heart to take over circulation on its own.

In so doing, not all the blood is passed through the unaffected heart, since-azygos factor, or azygos flow principle, notwithstanding—to eliminate all venous return to the affected heart if continued beyond a half hour would likely prove injurious. The level of immediate medical support provided much reduces the risk that either, much less both hearts would desynchronize. In fact, if the generally accepted explanation for the azygos factor is correct, that it evolved as a collateral or backup path to sustain venous return to the right atrium were either of the venae cavae to become obstructed, then azygos return added to the reduced venous return with one vena cava obstructed should sustain a patient indefinitely, or at least much longer than the time required to perform a less complex intracardiac repair under hypothermic conditions. In nature, however, the prospect for both venae cavae to be clamped with venous return completely closed off, as in open heart surgery, is remote.

With a significant reduction in load and the support of one another as well as the implant system, both hearts, initially impaired, would be strongly encouraged to recuperate, and without causing hyperperfusion, of which the added volume could immediately be shunted back into another large vessel through a diversion servovalve, for example. Even without adjustability, that two hearts allowed to recuperate would do so without adapting to the new condition, that is, relax in response to the reduction in load but instead persist in exerting the greater ejective force allowed by having recovered, and do so beyond the strength needed to drive the load, is contrary to experience of the heart as a component in an integrated system.

Where in vivo injury to a graft heart or other transplant organ had been secondary to a primary disorder, such as pulmonary hypertension, removal of the organ and its resituation in a body not so affected would reasonably be expected to allow for some recovery on that score alone, confrontation with new and different challenges, primarily immune intolerance, notwithstanding. Based upon the oversized heart as precedent, having been made integral in the circulatory system, the pair would respond to the load presented, not continue to develop needless strength as other than components in an integrated internally balanced system. It is, therefore, probable that the hearts would gain in fitness but not overdrive their loads.

While adherence to the setpoint established on the basis of the absolute stroke volume of either heart conserves energy, to apportion a close to normal venous retum volume to each heart, the servovalves on the venae cavae of both hearts are intermittently shifted in alternation to allow the one or the other to intake 80-90 percent of the total venous return. That double heart switch transplantation allows adjustment in the effective ejection fraction of the two hearts working together liberalizes the selection of donor hearts based upon size, thus considerably increasing the pool of acceptable donor hearts. Moreover, adjustability makes it possible to positively encourage recovery. Ventricular assist devices are also adjustable but when used CONTINUE POSTING EJ ADJUSTABILITY TO ADAPTATION

Where merged atria, or conventional heterotopic heart transplantation, was performed prior to the discovery of the first significant immunosuppressant cyclosporin to sustain circulation in the event of rejection, more recently, the procedure is performed in lieu of a heart-lung transplant to attenuate fixed or intractably elevated pulmonary hypertension resistant to medicinal reversal, and/or to overcome a significant size mismatch, as well as to expand the zone for usable hearts. In comparison, a double heart transplant, as addressed below in section 2b(1)(b)(b5), entitled Heterotopic Double Heart Metered Compound Vascular Bypass, or Switched-organ Transplantation [and so on], offers superior therapeutic versatility, survivability, and applicability, as well as further expands the zone of marginal hearts, that is, the pool of extended criteria donor hearts that can be accepted for transplant.

The transplantation of one or two hearts in less than ideal condition where neither has undergone the trauma of ischemia and extensive dissection is fundamentally superior to the placement of a heart, however intact from an anatomical standpoint, which had undergone circulatory death, or cardiorespiratory arrest, with ensuing asystole. Improved ex vivo reperfusion measures arose against a background of compulsory rejection of hearts that gave the outward appearance of adequacy despite having undergone severe endothelial injury. In relation to the preceding situation, during which such hearts were rejected, means for secondarily beneficiating hearts previously discarded was advantageous.

However, the use of hearts from the dead where the heart itself had already died is inferior to a heart that had never experienced ischemia and nonperfusion, not the least due to reperfusion injury when artificially reperfused prior to transplantation, but then extensively traumatized by having to undergo a bicaval operation. “Even though DCD [donated after cardiac death] hearts and lungs have been successfully transplanted, the risks associated with using these lower quality grafts is very high” (Bezinover, D. and Saner, F. 2019. “Organ Transplantation in the Modern Era,” Online, BioMed Central Anesthesiology 19(1): 32)(see also Chew, H. C., Macdonald, P. S., and Dhital, K. K. 2019. “The Donor Heart and Organ Perfusion Technology,” Journal of Thoracic Disease 11 (Supplement 6): S938-S945; Pinezich, M. and Vunjak-Novakovic, G. 2019. “Bioengineering Approaches to Organ Preservation ex Vivo,” Experimental Biology and Medicine (Maywood, New Jersey) 244(8): 630-645; Dhital, K. K., Iyer, A., Connellan, M., Chew, H. C., Gao, L., and 16 others 2015. “Adult Heart Transplantation with Distant Procurement and Ex-vivo Preservation of Donor Hearts after Circulatory Death: A Case Series,” Lancet 385(9987): 2585-2591; Fatullayev, J., Samak, M., Sabashnikov, A., Weymann, A., Mohite, P. N., and 6 others 2015. “Non-heart- beating Donor Heart Transplantation: Breaking the Taboo,” Medical Science Monitor Basic Research 21:153-156).

While an implanted sensor-driven automatic response system with direct fluid and electrical lines can be applied to any transplant to immediately and selectively pipeline-target a fluid drug or deliver electrical discharges to it, in a conventional heterotopic heart transplant, the hearts are merged, precluding this capability. By contrast, in a double heart transplant, each heart remains intact as to be separately targetable—the donor heart with an immunosuppressant, for example—sparing the native heart and all other tissues in the body from exposure to the drug or drugs. Not inextricably united as in a heterotopic transplant, each heart can be separately diagnosed, adjusted, treated, and should it become necessary, removed while the other heart sustained the recipient.

All immunosuppressants increase susceptibility to infection and produce adverse side effects. Cyclosporin, for example, is not only a carcinogen, but often causes hypertension, convulsions, nausea, kidney and/or liver dysfunction, dyspnea, paresthesia, gastrointestinal ulcers, hypercholesterolemia, along with several others which limit its dose when not targeted. Tightly targeted steroids, radionuclides, chemotherapeutics, and immunosuppressants can be delivered to a specific organ in the optimal dose without regard to side effects.

When necessary, such as to destroy malignant cells shed by a “mother” neoplasm, the systemically dispersed dose can be reduced while that directly targeted increased. The hearts are therefore separately treatable, with any deficit or malfunction as might affect the one not inextricably tied to the other. Side effects of drugs directly released into the carotids to treat a psychiatric disorder will likely eliminate the provocation of side effects elsewhere in the body but not side effects in the brain itself, such as tardive dyskinesia, and this relation probably holds in numerous instances where the target and the side effect originate in the same tissue.

Because a thoracic entry wound tends to be draining and painful for an impaired patient, reentry is discouraged, the implanted disorder response system and clinical measure used to the fullest and proven inadequate before the decision to reenter. The same applies to a need for removal and replacement, which should always be accomplished in a single operation. This understood, following a switched-organ heterotopic double heart transplant, if for any reason either heart must be removed, whether because it has become irreversibly diseased, materially gained in or lost competency, or that added shows signs of vasculopathy or rejection, the process for removal of either heart is inherent in the original switched-organ procedure.

Explantation of the accessory or removal of the primary heart and its replacement following a compound vascular bypass heterotopic double heart transplant whether placed in the chest or the abdomen

Secondary removal of the native or earlier transplanted heart corresponds to its removal in an orthotopic compound vascular bypass, or switched-organ heart transplant. As shown in FIG. 18B for its arterial and in FIG. 19B for its venous connections, in a sudden switch procedure, highly damped nonsparking plunger solenoid-driven valves are fully opened to bypass all blood passing through the primary heart to and through the corresponding vessels of the allograft, the vessels of the native heart then clamped and severed between the valves and the heart itself. This leaves the vessels of the allograft supplied and drained by the remaining corresponding vessels of the native heart.

The equivalent representations for a metered servovalve-driven double heart transplant, which interposes a stage of gradually increased acclimation between the native and allograft hearts as to require servovalves rather than bistable solenoid-driven valves, is shown in FIGS. 20C and 21C. Removal of the accessory, or added, heart is by closing the valves that had been used to pass blood to it, the accessory heart then clamped and severed between the valves and the heart itself. If contemplated for follow-up reconnection to a replacement heart, measures are taken to preserve the stumps as best might be accomplished by assuring their perfusion and using current filler materials. The stumps should provide a length sufficient for the placement of second valves should the need for transplant replacement arise as addressed below. By ‘primary heart’ is meant that native or previously transplanted.

With any type of heterotopic double heart transplant, the ability of the automatic disorder response system to automatically bypass and/or resynchronize either heart must be considered. Whereas in a heterotopic double heart transplant, the added heart is dependent upon the primary heart to pass blood to and from it, direct aortocaval connection provides relatively substantial independence of function between the two hearts. In a hybrid doubly heterotopic heart transplant, the connections between the hearts across the chest reconstitutes a measure of dependency upon the primary or native heart by that added.

The explantation of an accessory transplanted heart without its replacement is appropriate only when the native heart has recovered to the extent that it can continue to function without the assistance of a second heart and the fundamental decrease in the odds for adverse sequelae direct pipe-targeted delivery provides notwithstanding, the patient spared the need for immunosuppressive therapy and the risks of infection and malignancy it bodes. This is a prospect that can be expected to advance with further developments in cell and gene therapy. When necessary, a replacement is not deferred to a later procedure but rather follows the removal immediately.

More than one method can be used to replace the primary, that is, the native or earlier transplanted heart in a compound bypass heterotopic heart transplant. Following its removal, in FIG. 18B showing the arterial valve and FIG. 19B showing the venous valve, the servovalves remain positioned attached to and toward the ends of the arterial and venous stumps respectively. This having been a heterotopic switched-organ double heart transplant, prior to removal of the primary heart-depending upon the relative sizes of the primary and the latter added, or secondary heart—the valves would have been about half-way open. Just prior to removal of the secondary heart, the valves had to be adjusted to the fully diversion chute-extended, or fully open, position so that all arterial flow to and all venous flow from the primary heart would be cut off and passed to and through the secondary heart.

The donor of the allograft having been sustained on life support begun prior to expiration, the donor and recipient positioned beside one another, the arterial vessels less the pulmonary trunk of artery of the donor heart are connected to valves positioned closely above and below the plunger solenoid valves in a sudden switch procedure shown in FIG. 18B and the veins connected to valves positioned closely above and below the valves shown above in FIG. 19B, or more likely, in a metered switch procedure, the arterial valves in FIG. 20C and those venous in FIG. 21C.

The half-way opening of the added valves must be synchronous with the half-way retraction of the existing valves, thus dividing the volume of blood serving and ejected by either heart. Whether solenoid or servomotor driven all valves, or if basic fixed side-entry diversion jackets, incorporate at least one drugline to allow the direct pipeline-targeting of medication, to include immunosuppressive and antimicrobial, to and through each valve.

Another approach is to position the replacement heart by direct aortocaval connection as delineated below in section 2b(1)(b)(b17), entitled Doubly Heterotopic Double Heart Compound Vascular Bypass, or Switched-organ Transplantation, the graft organ then positioned to the right of that native in the chest by connection with elongated arterial and venous confluence conduits. Also considered is direct aortocaval connection with direct connection of the pulmonary vessels between the hearts across the chest as described below in section 2b(1)(b)(b18), entitled Hybrid Doubly Heterotopic Double Heart Transplantation.

Yet another approach calls for repositioning the accessory heart mediastinally to serve as a surrogate ‘native’ heart. The blood and druglines must be flexible and not kink to allow the move. Adhesions if encountered are addressed above in section 2a, entitled Background. General, in the subsection entitled Prevention of Adhesions. Such measures should have been taken at the time of implantation.

To replace the secondary (accessory, added) heart in a compound vascular bypass heterotopic double heart transplant, the added heart is cut off from blood flow by fully opening the valves of the primary heart so that no blood is diverted from it to the allograft. Referring to FIG. 8, this means the diversion chutes 18 are fully retracted causing obturators 30 to seal off the sidestem effluent channels. Blood now circulating through the primary heart, the bloodlines connected to the sidestems are disconnected and the accessory heart removed and saved for diagnosis or discarded, and the valves sterilized for reuse.

The plunger solenoid-driven vascular valve shown in FIG. 7 differs from that shown in FIG. 8 in presenting a shorter and postimplantation-removable portion of the valve below the sidestem 19 obtained by reversing the driver from a pusher to a puller. Shown only for the solenoid-driven valves, this variant is no less applicable to vascular servovalves where a miniature servomotor such as that shown in FIG. 9 is reversed in orientation relative to central axis of the jacket portion 3 of the servovalves. In the application shown in FIGS. 25A and 25B, the compactness of the anatomy requires that to avoid complications and discomfort to the patient, the servovalves be as small and light in weight as possible.

Whereas orthopedic prostheses which replace natural anatomy and inherently have a designated existing place, side-entry devices are neither bone cemented nor anchored implants for which nature would have made future allowance. Tissues in the neck, for example, are not simply adjacent but often fastened together by intervening connective tissue which may have to be teased apart or more decisively separated to create a space of which the walls have been desensitized or denervated to accommodate a spacer. The development of implantable spacers for placement in locations that afford little or inadequate space and especially means for ameliorating such restriction is virtually unaddressed in the literature. Spacers used in spinal neurosurgery to relieve the pain caused by compressed nerves are special purpose solid devices and not the kind of spacers meant here.

Where the mechanism of the implant and the need to match the size of the substrate does not impose a limit to the degree of miniaturization, the lack of adequate space is less of a problem (see, for example, Chiang, B., Jang, K., Goldberg, J., and Myung, D. 2024. “Design and Ex Vivo Development of a Suprachoroidal Spacer Implant to Treat Glaucoma,” Online, Research Square (Durham, North Carolina) preprint at https://www.researchsquare.com/article/rs-3895533/v1). The size of a side-entry jacket device is limited by the need for a flow diversion chute, part number 18 in the drawing figures, with a surface area and obturator, part number 30, integral with or fused to its front tip sufficient to completely close off continued flow through the substrate ductus.

Just how much smaller in diameter a diversion chute can be made than the lumen of the substrate ductus and still fully cut off all through-flow to divert it into a bloodline is primarily a problem for polymer chemistry and materials physics to determine. Additional measures to achieve spatial accommodation without minimal trauma or even discomfort to the patient are the latitude side-entry devices present as to the level and rotational angle for mounting valves and servovalves, for example; the elimination of sharp edges and corners; the application of an outer cushioning layer; and if necessary, the use of suture loops, part number 15 in the drawing figures. Until the suture-suture hole in the tissue to which the device is anchored fibroses, this is likely to cause the patient discomfort. As addressed just below, discomfort during healing can be counteracted through the use of time-released anesthetic—loaded or—coated suture.

Where it can be applied, another method to allow a more accurate positioning of a side-entry device is to avoid suture and directly fasten the substrate ductus to adjacent tissue (see, for example, Tsai, Y. C., Huang, D. W., Chou, Y. Y., An, Y. C., Cheng, Y. S., Chen, P. H., and Tzeng. Y. S. 2023. “Comparative Evaluation of Tissue Adhesives and Sutures in the Management of Facial Laceration Wounds in Children,” Online, MDPI [Multidisciplinary Digital Publishing Institute] Journal of Personalized Medicine (Basel, Switzerland) 13(9): 1350).

For this reason, the foregoing measures notwithstanding, where there is refractory resistance to gaining a position unobtrusive to neighboring tissue requiring the use of one or more suture loops likely to irritate the patient, the use of adjacent time-release anesthetic drug-eluting implants or time-release anesthetic such as lidocaine or bupivacaine, or an analgesic, and/or anti-inflammatory-loaded or coated suture to lasso or stitch to neighboring or nearby structure that remains in effect until fibrosis has been completed allows the operator to nudge the side-entry device into the position desired (see, for example, Khalid, G. M. and Billa, N. 2023. “Drug-eluting Sutures by Hot-melt Extrusion: Current Trends and Future Potentials,” Online, MDPI [Multidisciplinary Digital Publishing Institute] Materials (Basel, Switzerland) 16(22): 7245; Akombaetwa, N., Bwanga, A., Makoni. P. A., and Witika, B. A. 2022. “Applications of Electrospun Drug-eluting Nanofibers in Wound Healing: Current and Future Perspectives, Online, MDPI [Multidisciplinary Digital Publishing Institute] Polymers (Basel, Switzerland) 14(14): 2931; Xu. L., Liu, Y., Zhou, W., Yu. D. 2022. “Electrospun Medical Sutures for Wound Healing: A Review,” Online, MDPI [Multidisciplinary Digital Publishing Institute] Polymers (Basel, Switzerland) 14(9): 1637; Hakim, M. L., Nahar, N., Saha, M., Islam, M. S., Reza, H. M., and Sharker, S. M. 2020. “Local Drug delivery from Surgical Thread for Area-specific Anesthesia,” Biomedical Physics and Engineering Express (Bristol, England) 6(1): 015028; Kim, H., Kim, B. H., Huh, B. K., Yoo, Y. C., Heo. C. Y., Choy. Y. B., and Park, J. H. 2017. “Surgical Suture Releasing Macrophage-targeted Drug-loaded Nanoparticles for an Enhanced Anti-inflammatory Effect,” Biomaterials Science (Cambridge, England) 5(8): 1670-1677; Weldon, C. B., Tsui, J. H., Shankarappa, S. A., Nguyen, V. T., Ma, M., Anderson, D. G., and Kohane. D. S. 2012. “Electrospun Drug-eluting Sutures for Local Anesthesia,” Online, Journal of Controlled Release (Amsterdam, North Holland, Netherlands) 161(3): 903-909; Exner, A. A. and Saidel, G. M. 2008. “Drug-eluting Polymer Implants in Cancer Therapy,” Expert Opinion on Drug Delivery (London, England) 5(7): 775-88).

Other approaches to avoiding a protrusive or abrasive contact with neighboring tissue are to urge the substrate ductus into the position desired by attaching it to neighboring tissue with aid of tabs connected by suture and fastened at either end with an adhesive, and another where one point of attachment is bone is to use a suture anchor at that end. Where long skeletal muscles, for example, having been separated insist on resiliently recovering into adjacency, yet another method for creating a space between the two is by inserting a small pad of surgical gauze sponge loaded or coated with agents to prevent the development of retained object complications such as a gossypiborna with the development of a foreign body granuloma as a result of failing to remove the surgical sponge. Counterintuitively as is the method itself, here adhesions will not be between tissue desired separate but rather beneficial in providing positional stability. Prospectively new and useful in relation to side-entry devices as spacers, surgical sponges have yet to be produced for permanent implantation.

However, pinching closed hydroxyethyl cellulose/soy protein isolate or if to be absorbed once pain is less likely, polyglycolic acid (polyglycolide) sponge conduits can be used thus (see, for example. Dong, Q., Ai, J., Xiao, A., Wu, P., Wu, M., and 5 others 2023. “Defect Treatment with a Capping Hydroxyethyl Cellulose/Soy Protein Isolate Sponge Conduit for Painful Neuroma Prevention,” Online, ACS [American Chemical Society] Omega (Washington, D. C.) 8(34): 30850-30858)(see also Sisti, A., Uygur, S. Lopez-Schultz, S. D., and Konofaos, P. 2024. “Nerve Capping Techniques for Neuroma Management: A Comprehensive Literature Review,” Annals of Plastic Surgery (Boston, Massachusetts) 92(1): 106-119; Wan, T., Li, Q. C., Qin, M. Y., Wang, Y. L., Zhang, F. S., and 3 others 2024. “Strategies for Treating Traumatic Neuromas with Tissue-engineered Materials,” Online, MDPI [Multidisciplinary Digital Publishing Institute] Biomolecules (Basel, Switzerland) 14(4): 484; Onode, E., Uemura, T., Hama, S., Yokoi, T., Okada, M., Takamatsu, K., and Nakamura, H. 2022. “Nerve-end Capping Treatment with a Polyglycolic Acid Conduit for Rat Sciatic Neuroma: A Preliminary Report,” Journal of Reconstructive Microsurgery (New York, New York) 38(9): 711-720)

Muscle is capable of regeneration, and in an area such as the extremities where the nerves, vessels, and skeletal muscle are in direct adjacency, the resection of a minimal volume of muscle could provide the space needed (see, for example, Ahmad K, Shaikh S, Chun H J, Ali S, Lim J H, and 3 others 2023. “Extracellular Matrix: The Critical Contributor to Skeletal Muscle Regeneration—A Comprehensive Review,” Online, BioMed Central Inflammation and Regeneration (London, England) 43(1): 58; Anderson, J. E. 2022. “Key Concepts in Muscle Regeneration: Muscle “cellular ecology” Integrates a Gestalt of Cellular Cross-talk, Motility, and Activity to Remodel Structure and Restore Function,” Online, European Journal of Applied Physiology (Berlin, Germany) 122(2): 273-300; Alarcin, E., Bal-Öztürk, A., Avci, H., Ghorbanpoor, H., Dogan Guzel, F., and 4 others 2021. “Current Strategies for the Regeneration of Skeletal Muscle Tissue,” Online, MDPI [Multidisciplinary Digital Publishing Institute] International Journal of Molecular Sciences (Basel, Switzerland) 22(11): 5929; Qiu, X., Liu, S., Zhang, H., Zhu, B., Su. Y., and 6 others 2018. “Mesenchymal Stem Cells and Extracellular Matrix Scaffold Promote Muscle Regeneration by Synergistically Regulating Macrophage Polarization Toward the M2 Phenotype,” Online, BioMed Central Stem Cell Research and Therapy (London, England) 9(1): 88).

However, the form is as important as the essential fact of regeneration; deprived of its original volume due to the resection of lung tissue in order to allow the positioning of second heart in the chest, for example, lung tissue will regenerate by increasing the density of alveoli within the remaining space. It is essential that in recovering, the regenerated muscle not seek to reenter the space denied as would cause the patient discomfort if not pain. Based upon the existing literature, the form of muscle regeneration in contact with different neighboring tissues also warrants research. Because the advent of side-entry jacket devices creates the problem of minimizing protrusive extension into adjacent tissue in compact anatomy, no spacers have been devised and no research into prospective measure usable to alleviate any discomfort for the patient.

Accordingly, latitude in the level and rotational angle at which the jacket is attached in combination with minimizing the size and weight of the device and the application of an external cushioning layer must suffice. The abaxial portions of sidestem 19 and diversion 18 coursing along its bottom chute could be made pliant enough to be bent up or down along a track vertical and substantially parallel rather than perpendicular to the central axis of the side-entry valve jacket 3 and wall of the substrate ductus 2 to move along a curved track; however, this would only change the problem of protrusion into one of vertical rather than perpendicular protrusion.

Where the control of a stream is fixed as purely directional without the need for adjustment in the volume thereof, protrusion is eliminated through the use of a one-, two-, or three-way passive side-entry flow diversion jacket as described below in section 2b, entitled Vascular Valves and Servovalves in the Diversion of Blood Flow, in the subsection thereof entitled One-, Two-, and Three-way Passive Side-entry Flow Diversion Jackets. Lacking the need for a volume adjustability capability, these, as do inline coupling jackets, have no sidestem into which the flow diversion chute can be withdrawn.

The paramount object in devising side-entry devices and inline coupling jacket-secured tie-lines is to impart the ability to draw off all or a continuously variable fraction of the flow through a ductus, most often a blood vessel, for continued flow through a synthetic line without the need to transect and thereby eliminate the need to interrupt flow through the ductus which would undergo anoxia, in itself a major cause for complications in cardiovascular procedures. The only way to accomplish this is to enter through an opening in the side of the ductus with an extendable and retractable tap-tongue having a flow diverting distal end curved in the direction opposite to the flow. This connotes that some degree of protrusion outside the ductus will be unavoidable.

The recipient and donor of the replacement secondary heart positioned side by side, sterile valves are attached to the vessels of the prospective graft and the bloodlines extending from these connected to the sidestems 19 of the valves on the primary heart. FIGS. 18A and 18B show the harvesting of the replacement heart in a sudden-switch procedure, and FIGS. 19A and 19B show this in a metered procedure. Replacement of the added heart thus retraces the steps taken to transplant the original second heart.

Replacement of both the primary and accessory hearts is by replacing one at a time in the same procedure that removed both. While both hearts in a double heart patient can be replaced in the same procedure in succession, the replacement of the native heart, the native heart with a transplanted accessory, that is, added, or double heart, or both a primary and secondary transplanted hearts is not by replacement of both hearts together, that is, simultaneously.

As delineated below in section 2b(b5) entitled Double Heart Metered Compound vascular bypass, or sudden switched-organ Transplantation, for physiological reasons, the donor heart is preferably positioned adjacent to that native to its right-hand side in the chest. To spare the patient additional pain, and to sustain circulation without the need or with a reduced need for mechanical support, procedures that might be performed in successive operations should be completed in a single operation. This includes the removal and replacement of both hearts and when necessary, the resection of the lower right lung to create space for an accessory heart in the chest.

### Explantation of the Accessory or Removal of the Primary Heart and its Replacement Following a Compound Vascular Bypass Doubly Heterotopic Double Heart Transplant

The added heart in a heterotopic double heart transplant is positioned in the lower abdomen without aortocaval connection, so that its connections to the elongated arterial and venous confluence conduits are between the two hearts and not to the abdominal aorta and inferior vena cava. Accordingly, its connections are the same as specified above for a heterotopic double heart transplant with the second heart positioned in the chest, where the elongated arterial and venous confluence conduits may be thought of as ‘extension cords.’ A doubly heterotopic compound vascular bypass double heart transplant involves no connections between the native or earlier transplanted, that is, the primary heart, and the added, or secondary, heart.

This attests not only to the independence between the hearts to the extent that the added heart does not receive and return its blood to the primary heart making the accessory heart dependent upon it for synchronized function. Accordingly, its connection, removal, and replacement are comprehended in the foregoing discussion pertaining to removals and replacements in compound vascular bypass heterotopic double heart transplants. Unrelated thereto, connections of the elongated arterial and venous confluence conduits to the abdominal aorta and inferior vena cava are addressed above in connection with doubly heterotopic and hybrid doubly heterotopic heart transplantation.

### Explantation of the Accessory or Removal of the Primary Heart and its Replacement Following a Compound Vascular Bypass Hybrid Doubly Heterotopic Double Heart Transplant

Unlike conventional heterotopic heart transplantation in which the hearts are combined, the hearts are separate, so that the implanted sensor-driven automatic response system with direct fluid and electrical lines can target either heart for medication and/or electrostimulation. With a native heart nearing end stage failure, that is, at American College of Cardiology Stage D or New York Heart Association Class IV, adding a second heart not only assists that native by alleviating most of its load, but in so doing, affords the native heart the opportunity to recover in situ with the aid of medication selectively targeted to it automatically as necessary.

Should the reduction in intracardiac blood volume precipitate a dysrhythmia in either or both hearts, the implant microcontroller or microprocessor, based upon the input data stream received from implantable heart beat sensor circuits, or pacemaker/cardioverter defibrillator detection and control timing circuits as sensors, can alternate the volume of blood processed by either heart. That is, rather than to sustain the diversion chute settings at the apportionment set on the basis of relative absolute stroke volumes of either heart, the chutes are moved to alternately apportion almost all of the blood volume to either in turn, the rate and extent of alternate volume apportionment determined by the controller executing an algorithm that locates the pattern of apportionment which best yields rhythmical durability.

In practice, the hearts should intermittently beat together; however, this should be brief, and as both are likely impaired, the sum stroke volume, or ejection fraction, during these brief intervals should not result in pulmonary resistance. The controller is also able to adjust the dose of dysrhythmic medication and deliver resynchronizing optogenetic or electrostimulation. Thus, should cardiac allograft coronary artery vasculopathy impair the transplanted heart or hearts, the unaffected native or less affected cardiac allograft would continue circulation, and given that vasculopathy does not ensue for years following implantation, the unaffected native if not the allograft would have had time to recover function it had lost prior to implantation, much reducing the prospect of a sudden cardiac arrest.

Were either heart to become infected, rejected, or otherwise impaired, the backup would sustain the patient long enough for transport to a hospital. The best-case outcome is that the native heart would have recovered sufficiently to sustain the patient indefinitely. If neither heart were able to serve the patient, then the time gained would allow a medical response. Post-transplantation development of vasculopathy is addressed below in this and other sections directed to transplantation of the heart.

In comparison with the causes for cardiac allograft vasculopathy, the risk factors that promote its development-advanced age, smoking, obesity, hypertension, dyslipidemia, and so on are well established and mostly preventable (see, for example, Szygula-Jurkiewicz, B., Szczurek, W., Gąsior, M., and Zembala, M. 2015, Op cit.; Colvin-Adams, M and Agnihotri, A. 2011. “Cardiac Allograft Vasculopathy: Current Knowledge and Future Direction” Clinical Transplantation 25(2): 175-184).

A probable—and if so, critical—benefit of switch heart transplantation is that to the extent vasculopathy as the cause of failure in half of all heart transplants—appears at least in part as a late result of the cytokine storms associated with the death of the donor, cessation of function, excision, and ischemia usually extended throughout preservation in cold storage, followed by the ischemia and trauma of conventional transplantation, the elimination of these factors should result in an attenuation in if not complete avoidance of vasculopathy, and therewith, the prelimitation in service life of the new heart it imposes. The literature on cardiac allograft vasculopathy is directed to its treatment, not prevention.

Given that a heart transplant supported with statins, platelet blockers, antiproliferatives such as sirolimus and everolimus, with other drugs responsive to particulars such as antihypertensives, anti-inflammatories, antimicrobials, and desynchronization counteracting electronics, the elimination of ischemia-associated trauma attendant upon procurement and handling of the graft should allow a significant retardation if not elimination in cardiac allograft vasculopathy to allow the recipient a longer and better quality of life.

“Cardiac allograft vasculopathy prevention may involve therapy that provides protection against endothelial injury implemented just before transplantation, during storage and transplantation as well as after transplantation” (Volpe, M., Rubattu. S., and Battistoni, A. 2019. “ARNi [angiotensin receptor-neprilysin inhibitors]: A Novel Approach to Counteract Cardiovascular Diseases,” Online, MDPI [Multidisciplinary Digital Publishing Institute] International Journal of Molecular Sciences (Basel, Switzerland) 20(9). pii: E2092); (see also, Peyster, E. G., Janowczyk. A., Swamidoss. A., Kethireddy, S., Feldman, M. D., and Margulies, K. B. 2022. “Computational Analysis of Routine Biopsies Improves Diagnosis and Prediction of Cardiac Allograft Vasculopathy,” Online, Circulation (Hagerstown, Maryland) 145(21): 1563-1577; Szczurek-Wasilewicz, W., Hawranek, M., Skrzypek, M., Hrapkowicz, T., Gąsior, M., Warmusz, O., and Szyguła-Jurkiewicz, B. 2022. “Factors Associated with Cardiac Allograft Vasculopathy after Heart Transplantation,” Online in English, Postepy Kardiol Interwencyjnej [Advances in Interventional Cardiology.] (Poznań, Poland) 18(3): 237-245; Szczurek, W., Gąsior, M., Romuk, E., Skrzypek, M., Zembala, M., and Szyguła-Jurkiewicz B. 2020. “Investigation of the Role of Oxidative Stress and Factors Associated with Cardiac Allograft Vasculopathy in Patients after Heart Transplantation,” Online, Oxidative Medicine and Cellular Longevity (New York, New York) 2020:7436982; Volterrani, M., Iellamo, F., Senni, M., and Piepoli, M. F. 2017. “Therapeutic Options of Angiotensin Receptor Neprilysin Inhibitors (ARNis) in Chronic Heart Failure with Reduced Ejection Fraction: Beyond RAAS [renin-angiotensin-aldosterone system] and Sympathetic Nervous System Inhibition,” International Journal of Cardiology 226:132-135; Szygula-Jurkiewicz, B., Szczurek, W., Gąsior, M., and Zembala, M. 2015. “Risk Factors of Cardiac Allograft Vasculopathy,” Kardiochirurgia i Torakochirurgia Polska (Polish Journal of Cardiac and Thoracic Surgery) 12(4): 328-333; Seki. A. and Fishbein, M. C. 2014. “Predicting the Development of Cardiac Allograft Vasculopathy,” Cardiovascular Pathology (New York, New York) 23(5): 253-260; Skoric, B., Čikeš. M., Ljubas Maček, J., Baričević, Ž., Škorak, I., and 3 others 2014. “Cardiac Allograft Vasculopathy: Diagnosis, Therapy, and Prognosis,” Croatian Medical Journal 55(6): 562-576; Ramzy, D., Rao, V., Brahm, J., Miriuka, S., Delgado, D., and Ross, H. J. 2005. “Cardiac Allograft Vasculopathy: A Review,” Online, Canadian Journal of Surgery (Ottawa, Canada) 48(4): 319-327; Fateh-Moghadam, S., Bocksch, W., Ruf. A., Dickfeld, T., Schartl. M., and 4 others 2000. “Changes in Surface Expression of Platelet Membrane Glycoproteins and Progression of Heart Transplant Vasculopathy,” Circulation 102(8): 890-897; additional references to this and related subjects appear in sections to follow). Because the denervated heart precludes the sensation of angina responsive to atherosclerotic occlusion, the onset of impending graft failure due to vasculopathy is either made evident by a diagnosis of heart failure or is caught during periodic follow-up imaging.

In fact, the injury imposed upon the graft organ is considerably greater than that done to its endothelium. In eliminating the trauma associated with death, excision from the body from which, while remaining under life support, it is properly inseparable, ischemia, cold storage, extensive dissection, reperfusion injury, then fixation within an alien repellent milieu, the combination of life support and switch methodology should materially improve outcomes in solid organ transplantation Other causes are the immune response to the allograft and immunosuppressants, in addition to hyperlipidemia, insulin resistance, hypertension, infection, and/or hypertension if present.

Where atherosclerosis is largely shear stress-driven mechanically as to affect larger arteries of high flow rate especially at bifurcations while sparing arterioles and capillaries, cardiac allograft vasculopathy, albeit differently, affects all segments. “A lack of correlation between microvascular and epicardial vessel disease suggests discordant manifestations and progression of CAV” (Weis, M. and von Scheidt, W. 1997. “Cardiac Allograft Vasculopathy: A Review,” Circulation 96(6): 2069-2077), even resulting in atrophy and loss of terminal microvasculature, or “pruning” (Lee, M. S., Tadwalkar, R. V., Fearon, W. F., Kirtane, A. J., Patel, A. J., and 3 others 2018. “Cardiac Allograft Vasculopathy: A Review,” Catheterization and Cardiovascular Interventions 92(7): E527-E536, Op. cit. above in section Background. General); (see also, for example, Forte, M., Madonna, M., Schiavon, S., Valenti, V., Versaci, F., and 3 others 2019. “Cardiovascular Pleiotropic Effects of Natriuretic Peptides,” Online, MDPI [Multidisciplinary Digital Publishing Institute] International Journal of Molecular Sciences (Basel, Switzerland) 20(16). pii: E3874; Volpe, M., Rubattu, S., and Battistoni, A. 2019, Op cit. second paragraph above; Stehlik, J., Kobashigawa, J., Hunt, S. A., Reichenspurner, H., and Kirklin, J. K. 2018. “Honoring 50 Years of Clinical Heart Transplantation in Circulation: In-depth State-of-the-art Review,” Circulation 137(1): 71-87; Klein, R., Sharrett, A. R., Klein, B. E., Chambless, L. E., Cooper, L. S., Hubbard, L. D., and Evans, G. 2000. “Are Retinal Arteriolar Abnormalities Related to Atherosclerosis? The Atherosclerosis Risk in Communities Study,” Arteriosclerosis, Thrombosis, and Vascular Biology 20(6): 1644-1650; Weis. M. and von Scheidt, W. 1997. “Cardiac Allograft Vasculopathy: A Review,” Circulation 96(6): 2069-2077; additional references cited below in section 2b(1)(a), entitled Sudden Compound Vascular Bypass, or Sudden Switched-organ Transplantation, for example).

While the effect can take as long as ten years, cardiac allograft vasculopathy eventually necessitates the retransplantation of about half of conventional, or caval orthotopic, heart transplants. However, with the tolerance induction of metered switch heart transplantation and follow-up provided by the implant automatic response system to include the direct targeting of drugs to any part of the heart the incidence of vasculopathy as a late-term cause of sudden death should be considerably reduced (Kleinmahon, J. A., Gralla, J., Kirk, R., Auerbach, S. R., Henderson, H. T., and 6 others 2019. “Cardiac Allograft Vasculopathy and Graft Failure in Pediatric Heart Transplant Recipients after Rejection with Severe Hemodynamic Compromise,” Journal of Heart and Lung Transplantation (New York, New York) 38(3): 277-284; Chen, Z., Pazdernik, M., Zhang, H., Wahle, A., Guo, Z., and 5 others 2018. “Quantitative 3D Analysis of Coronary Wall Morphology in Heart Transplant Patients: OCT [optical coherence tomography]—Assessed Cardiac Allograft Vasculopathy Progression,” Medical Image Analysis 50:95-105; Clemmensen, T. S., Holm, N. R., Eiskjær, H., Jakobsen, L., Berg. K., and 7 others 2018. “Detection of Early Changes in the Coronary Artery Microstructure after Heart Transplantation: A Prospective Optical Coherence Tomography Study,” Journal of Heart and Lung Transplantation (New York, New York) 37(4): 486-495; Flore. V., Brown, A. J., Pettit, S., West. N. E. J., Lewis, C., Parameshwar, J., and Hoole, S. P. 2018. “Intravascular Ultrasound of the Proximal Left Anterior Descending Artery is Sufficient to Detect Early Cardiac Allograft Vasculopathy,” Clinical Transplantation 32(2); Glinton, K., DeBerge, M., Yeap, X. Y., Zhang, J., Forbess, J., Luo, X., and Thorp E. B. 2018. “Acute and Chronic Phagocyte Determinants of Cardiac Allograft Vasculopathy,” Seminars in Immunopathology 40(6): 593-603; Goekler, J., Zuckermann, A., Kaider, A., Angleitner, P., Osorio-Jaramillo, E., and 6 others 2018. “Diminished Impact of Cytomegalovirus Infection on Graft Vasculopathy Development in the Antiviral Prophylaxis Era—A Retrospective Study,” Transplant International 31(8): 909-916; Pazdernik, M., Chen, Z., Bedanova, H., Kautzner, J., Melenovsky. V., and 10 others 2018. “Early Detection of Cardiac Allograft Vasculopathy Using Highly Automated 3-dimensional Optical Coherence Tomography Analysis,” Journal of Heart and Lung Transplantation (New York, New York) 37(8): 992-1000; Tadwalkar. R. V., Fearon, W. F., Kirtane, A. J., Patel, A. J., and 3 others 2018. “Cardiac Allograft Vasculopathy: A Review,” Catheterization and Cardiovascular Interventions 92(7): E527-E536; Zanchin, C., Yamaji, K., Rogge, C., Lesche, D., Zanchin, T., and 5 others 2018. “Progression of Cardiac Allograft Vasculopathy Assessed by Serial Three-vessel Quantitative Coronary Angiography,” Online, PLoS [Public Library of Science] One 13(8): e0202950; Clemmensen, T. S., Holm, N. R., Eiskjær, H., Logstrup, B. B., Christiansen, E. H., and 5 others 2017. “Layered Fibrotic Plaques are the Predominant Component in Cardiac Allograft Vasculopathy: Systematic Findings and Risk Stratification by OCT [optical coherence tomography],” Journal of the American College of Cardiology. Cardiovascular Imaging 10(7): 773-784; Hunt, S. A. 2017. “Cardiac Allograft Vasculopathy: It Really has Changed over Time,” Journal of the American College of Cardiology. Heart Failure 5(12): 902-903; Tremblay-Gravel, M., Racine, N., de Denus, S., Ducharme, A., Pelletier, G. B., and 6 others 2017. “Changes in Outcomes of Cardiac Allograft Vasculopathy over 30 Years following Heart Transplantation,” Journal of the American College of Cardiology. Heart Failure 5(12): 891-901; Burke, M. M. 2016. “Late Cardiac Allograft Failure, Cardiac Allograft Vasculopathy, and Antibody-mediated Rejection: Untangling Some Knots?,” American Journal of Transplantation 16(1): 9-10; Chih, S., Chong, A. Y., Mielniczuk, L. M., Bhatt, D. L., and Beanlands, R. S. 2016. “Allograft Vasculopathy: The Achilles' Heel of Heart Transplantation,” Journal of the American College of Cardiology 68(1): 80-91: Édes. I. F., Hajas, A., Sax, B., Bartykowszki, A., Becker, D., and Merkely, B. 2016. “Cardiac Allograft Vasculopathy: Optical Coherence Guided Innovative Treatment Options with the Bioresorbable Vascular Scaffold: Proof of Concept,” Afinerva Cardioangiologica 64(4): 487-493; Javaheri, A., Saha, N., and Lilly, S. M. 2016. “How to Approach the Assessment of Cardiac Allograft Vasculopathy in the Modern Era: Review of Invasive Imaging Modalities” Current Heart Failure Reports 13(2): 86-91; Loupy, A., Toquet, C., Rouvier, P., Beuscart, T., Bories, M. C., and 9 others 2016. “Late Failing Heart Allografts: Pathology of Cardiac Allograft Vasculopathy and Association with Antibody-mediated Rejection,” American Journal of Transplantation 16(1): 111-120; Tomai, F., De Luca, L., Petrolini, A., Di Vito, L., Ghini, A. S., and 6 others 2016. “Optical Coherence Tomography for Characterization of Cardiac Allograft Vasculopathy in Late Survivors of Pediatric Heart Transplantation,” Journal of Heart and Lung Transplantation (New York, New York) 35(1): 74-79; Delgado, J. F., Reyne, A. G., de Dios, S., Lopez-Medrano, F., Jurado, A., and 6 others 2015. “Influence of Cytomegalovirus Infection in the Development of Cardiac Allograft Vasculopathy after Heart Transplantation,” Journal of Heart and Lung Transplantation (New York, New York) 34(8): 1112-1119; Johansson, I., Andersson, R., Friman, V., Selimovic, N., Hanzen, L., and 3 others 2015. “Cytomegalovirus Infection and Disease Reduce 10-year Cardiac Allograft Vasculopathy-free Survival in Heart Transplant Recipients,” Online, BioMed Central Infectious Diseases 15:582).

The support of another heart sustains the patient past failure of either heart and likely both allows the native heart to recover as well as reduces the severity of sequelae. When a second heart is present, in addition to late-term vasculopathy, a sudden cardiac arrest, myocardial infarction, graft failure, dysrhythmia, or infection, for example, in either the native or earlier orthotopically transplanted, or in the added heart need not equate to imminent or sudden cardiac death (see, for example, Alba, A. C., Fan, C. S., Manlhiot, C., Dipchand, A. I., Stehlik, J., and Ross, H. J. 2019. “The Evolving Risk of Sudden Cardiac Death after Heart Transplant. An Analysis of the ISHLT [International Society for Heart and Lung Transplantation] Thoracic Transplant Registry,” Clinical Transplantation 33(3): e13490; Avtaar Singh, S. S., Banner, N. R., Rushton, S., Simon, A. R., Berry, C., and Al-Attar, N. 2019. “ISHLT [International Society for Heart and Lung Transplantation] Primary Graft Dysfunction Incidence, Risk Factors, and Outcome: A UK National Study,” Transplantation 103(2): 336-343; Ludhwani. D., Fan, J., and Kanmanthareddy, A. 2019. “Heart Transplantation Rejection,” Online at https://www.ncbi.nlm.nih.gov/books/NBK537057/; Mowers, K. L., Simpson, K. E., Gazit. A. Z., Eghtesady, P., Canter, C. E., and Castleberry, C. D. 2019. “Moderate-severe Primary Graft Dysfunction after Pediatric Heart Transplantation,” Pediatric Transplantation 23(2): e13340; Pons, S., Sonneville, R., Bouadma, L., Styfalova, L., Ruckly, S., and 9 others 2019. “Infectious Complications following Heart Transplantation in the Era of High-priority Allocation and Extracorporeal Membrane Oxygenation,” Annals of Intensive Care 9(1): 17; Singh, S. A., De, S. D., Rushton, S., Berry, C., and Al-Attar, N. 2019. “PREDICTA [a name, not an acronym]-A Model to Predict Primary Graft Dysfunction after Adult Heart Transplantation in the United Kingdom,” Journal of Cardiac Failure July pii: S1071-9164(19) 30138-1: Tsuji, M., Nitta. D., Minatsuki, S., Maki, H., Hosoya, Y., and 7 others 2019. “Emergency Percutaneous Coronary Intervention for Left Main Trunk Thrombus following Orthotopic Heart Transplantation,” European Society of Cardiology Heart Failure 6(3): 575-578; Alba, A. C., Foroutan, F., Ng Fat Hing. N. K. V., Fan, C. S., Manlhiot, C., and Ross, H. J. 2018. “Incidence and Predictors of Sudden Cardiac Death after Heart Transplantation: A Systematic Review and Meta-analysis,” Clinical Transplantation 32(3): e13206; Harris, C., Cao, C., Croce, B., and Munkholm-Larsen, S. 2018. “Heart Transplantation,” Annals of Cardiothoracic Surgery 7(1): 172; Kim, I. C., Youn, J. C., and Kobashigawa, J. A. 2018. Op cit., above in section 2a, entitled Background of the Invention. General: Lee, M. S., Lopez-Sainz, A., Barge-Caballero, E., Barge-Caballero, G., Couto-Mallon. D., Paniagua-Martin, M. J., and 6 others 2018. “Late Graft Failure in Heart Transplant Recipients: Incidence, Risk Factors and Clinical Outcomes,” European Journal of Heart Failure 20(2): 385- 394; Nicoara, A., Ruffin, D., Cooter, M., Patel, C. B., Thompson, A., and 10 others 2018. “Primary Graft Dysfunction after Heart Transplantation: Incidence, Trends, and Associated Risk Factors,” American Journal of Transplantation 18(6): 1461-1470; McCartney, S. L., Patel, C., and Del Rio, J. M. 2017. “Long-term Outcomes and Management of the Heart Transplant Recipient,” Best Practice and Research. Clinical Anesthesiology 31(2): 237-248; Squiers, J. J., Saracino, G., Chamogeorgakis, T., MacHannaford, J. C., Rafael, A. E., and 4 others 2017. “Application of the International Society for Heart and Lung Transplantation (ISHLT) Criteria for Primary Graft Dysfunction after Cardiac Transplantation: Outcomes from a High-volume Centre,” European Journal of Cardiothoracic Surgery 51(2): 263-270; Braulio, R., Sanches, M. D., Teixeira Junior, A. L., Costa, P. H., Moreira Mda, C., and 3 others 2016. “Associated Clinical and Laboratory Markers of Donor on Allograft Function after Heart Transplant,” Brazilian Journal of Cardiovascular Surgery 31(2): 89-97; D'Addio, F., Margonato, D., Pensato, U., Borgese. L., Potena. L., and Fiorina, P. 2015. “Novel Therapeutic and Diagnostic Management of Heart Transplant Patients,” Heart, Lung, and Vessels 7(3): 198-207Dronavalli, V. B., Rogers, C. A., and Banner, N. R. 2015. “Primary Cardiac Allograft Dysfunction-Validation of a Clinical Definition” Transplantation 99(9): 1919-1925; Vakil, K., Taimeh, Z., Sharma, A., Abidi, K. S., Colvin, M., and 3 others 2014. “Incidence, Predictors, and Temporal Trends of Sudden Cardiac Death after Heart Transplantation,” Heart Rhythm 11(10): 1684-1690).

The advantage in switch transplantation in not requiring an interruption in functioning of the heart to be transplanted and therefore not requiring the heart to be bypassed so that cardiopulmonary support under general anesthesia is necessary may be discerned in coronary artery bypass grafting. When compared to machine supported coronary artery bypass grafting, off-pump, or beating-heart coronary bypass has long been known to yield a better outcome. As indicated, the means of life support whether in a living patient receiving a prosthetic section of the thoracic, thoracoabdominal, or abdominal aorta here is not by conventional cardiopulmonary bypass which is intended to bypass the heart and lungs but rather through the use of a pacemaker-defibrillator.

“Off-pump coronary artery bypass was associated with a significant reduction in risk of death, stroke, acute renal failure, mortality or morbidity, and postoperative length of stay compared with on-pump coronary artery bypass after adjustment for 30 patient risk factors in the overall sample,” (Polomsky, M., He, X., O'Brien, S. M., and Puskas, J. D. 2013. “Outcomes of Off-pump Versus On-pump Coronary Artery Bypass Grafting: Impact of Preoperative Risk,” Journal of Thoracic and Cardiovascular Surgery 145(5): 1193-1198); (see also Chivasso, P., Guida, G. A., Fudulu, D., Bruno, V. D., and Marsico, R. 2016. “Impact of Off-pump Coronary Artery Bypass Grafting on Survival: Current Best Available Evidence,” Journal of Thoracic Disease 8 (Supplement 10): S808-S817)(see also Deppe, A. C., Arbash, W., Kuhn, E. W., Slottosch, I., Scherner. M., and 3 others 2016. “Current Evidence of Coronary Artery Bypass Grafting Off-pump Versus On-pump: A Systematic Review with Meta-analysis of Over 16,900 Patients Investigated in Randomized Controlled Trials,” European Journal of Cardiothoracic Surgery 49(4): 1031-1041; Ueki, C., Miyata, H., Motomura, N., Sakaguchi, G., Akimoto, T., and Takamoto, S. 2016. “Off-pump Versus On-pump Coronary Artery Bypass Grafting in Patients with Left Ventricular Dysfunction,” Journal of Thoracic and Cardiovascular Surgery 151(4): 1092-1098; Yanagawa. B., Nedadur, R., and Puskas, J. D. 2016. “The Future of Off-pump Coronary Artery Bypass Grafting: A North American Perspective,” Journal of Thoracic Disease 8 (Supplement 10): S832-S838; Carmona, P., Paredes, F., Mateo, E., Mena-Duran, A. V., Homero, F., and Martinez-Leon, J. 2016. “Is Off-pump Technique a Safer Procedure for Coronary Revascularization? A Propensity Score Analysis of 20 years of experience,” Interactive Cardiovascular and Thoracic Surgery 22(5): 612-618; Kowalewski, M., Pawliszak, W., Malvindi, P. G., Bokszanski, M. P., Perlinski, D., and 9 others 2016. “Off-pump Coronary Artery Bypass Grafting Improves Short-term Outcomes in High-risk Patients Compared with On-pump Coronary Artery Bypass Grafting: Meta-analysis,” Journal of Thoracic and Cardiovascular Surgery 151(1): 60-77; Keeling, W. B., Kilgo, P. D., Puskas, J. D., Halkos, M. E., Lattouf. O. M., Guyton, R. A., and Thourani, V. H. 2013. “Off-pump Coronary Artery Bypass Grafting Attenuates Morbidity and Mortality for Patients with Low and High Body Mass Index,” Journal of Thoracic and Cardiovascular Surgery 146(6): 1442-1448; Emmert, M. Y., Seifert. B., Wilhelm, M., Grunenfelder, J., Falk, V., and Salzberg, S. P. 2011. “Aortic No-touch Technique Makes the Difference in Off-pump Coronary Artery Bypass Grafting,” Journal of Thoracic and Cardiovascular Surgery 142(6): 1499-506).

More favorable outcomes with off-pump technique are experienced no less with intracardiac and combination coronary-intracardiac procedures (see, for example, Liu, J. J., Ren. C. W., Wu, W. H., and Lai, Y. Q. 2016. “One-stage Hybrid Procedure for Patients with Valvular Pulmonary Stenosis and Coronary Artery Disease,” Chinese Medical Journal (in English) 129(5): 624-625; Gillespie, M. J., Aoki, C., Takebayashi, S., Shimaoka, T., McGarvey, J. R., Gorman, R. C., and Gorman, J. H. 3rd 2015. “Development of Off-pump Mitral Valve Replacement in a Porcine Model,” Annals of Thoracic Surgery 99(4): 1408-1412; Thourani, V. H., Keeling, W. B., Guyton, R. A., Dara, A., Hurst, S. D., and Lattouf. O. M. 2011. “Outcomes of Off-pump Aortic Valve Bypass Surgery for the Relief of Aortic Stenosis in Adults,” Annals of Thoracic Surgery 91(1): 131-136).

Improvements in equipment, surgical technique, and patient selection have ameliorated some of the adverse consequences and elevated the value of cardiopulmonary support with respect to coronary artery bypass grafting (see, for example, Calafiore, A. M., Prapas. S., Osman, A., and Di Mauro, M. 2017. “Coronary Artery Bypass Grafting Off-pump or On-pump: Another Brick in the Wall,” Annals of Translational Medicine 5(7): 168; King, N. 2017. “On vs. Off Pump Coronary Artery Bypass Grafting: The Next Chapter,” Annals of Translational Medicine 5(5): 116; Kirmani, B. H., Holmes, M. V., and Muir, A. D. 2016. “Long-term Survival and Freedom from Reintervention after Off-pump Coronary Artery Bypass Grafting: A Propensity-matched Study,” Circulation 134(17): 1209-1220); however, pumpless heart transplantation has not been an option. Given teams in large centers with much experience using both techniques, off-pump coronary artery bypass grafting yields better outcomes (Chivasso, P., Guida, G. A., Fudulu, D., Bruno, V. D., and Marsico, R. 2016, Op. cit.).

The same guidelines as pertain to the qualifying conditions and preparation for the recipient and donor and follow-up for the recipient apply as usual. Postoperative care includes immunosuppression and the retarding of cardiac allograft vasculopathy—a factor that uninterrupted perfusion as well as periprocedural tolerance induction might reduce even with the metabolic syndrome with its hyperlipidemia as a mechanism of rejection (see, for example, Spartalis, M., Spartalis, E., Tzatzaki, E., Tsilimigras, D. I., Moris, D., and 4 others 2019. “Cardiac Allograft Vasculopathy after Heart Transplantation: Current Prevention and Treatment Strategies,” European Review for Medical and Pharmacological Sciences 23(1): 303-311; Sudharshan, S. and Javaheri, A. 2018. “Cholesterol Efflux in the Transplant Patient,” Current Opinion in Endocrinology, Diabetes, and Obesity 25(2): 143-146; Merola, J., Jane-Wit. D. D., and Pober, J. S. 2017. “Recent Advances in Allograft Vasculopathy,” Current Opinion in Organ Transplantation 22(1): 1-7; Bagley, J., Yuan, J., and Iacomini, J. 2017. “Impact of Hyperlipidemia on Alloimmunity,” Current Opinion in Organ Transplantation 22(1): 14-21; Ma. S., Jiang. Y., Huang, W., Li, X., and Li, S. 2017. “Role of Transient Receptor Potential Channels in Heart Transplantation: A Potential Novel Therapeutic Target for Cardiac Allograft Vasculopathy,” Medical Science Monitor 23:2340-2347; Chih, S., Chong, A. Y., Mielniczuk, L. M., Bhatt, D. L., Beanlands. R. S. 2016. “Allograft Vasculopathy: The Achilles' Heel of Heart Transplantation,” Journal of the American College of Cardiology 68(1): 80-91; Valenzuela, N. M., Hickey, M. J., and Reed, E. F. 2016. “Antibody Subclass Repertoire and Graft Outcome Following Solid Organ Transplantation,” Online, Frontiers in Immunology (Lausanne, Switzerland) 7:433; Kushwaha, S. and Hebl, Virginia 2016. “Sirolimus to Prevent Vasculopathy after Heart Transplant,” at http://www.medscape.com/viewarticle/858953; Qin, L., Li, G., Kirkiles-Smith, N., Clark, P., Fang. C., and 6 others 2016, Op. cit.; Horiuchil, T. and Tsukamoto, H. 2016, Op. cit.; Javaheri, A., Molina, M., Zamani, P., Rodrigues, A., Novak, E., and 17 others 2016. “Cholesterol Efflux Capacity of High-density Lipoprotein Correlates with Survival and Allograft Vasculopathy in Cardiac Transplant Recipients,” Journal of Heart and Lung Transplantation (New York, New York) 35(11): 1295-1302; Galli, G., Caliskan, K., Balk, A. H., van Domburg, R., Birim, O., Salerno-Uriarte, J., Manintveld, O. C., and Constantinescu, A. A. 2016. “Cardiac Allograft Vasculopathy in Dutch Heart Transplant Recipients,” Netherlands Heart Journal 24(12): 748-757; Yuan, J., Bagley, J., and Iacomini, J. 2015. “Hyperlipidemia Promotes Anti-donor Th17 Responses that Accelerate Allograft Rejection,” American Journal of Transplantation 15(9): 2336-2345; Bagley, J., Yuan, J., Chandrakar, A., and Iacomini, J. 2015. “Hyperlipidemia Alters Regulatory T Cell Function and Promotes Resistance to Tolerance Induction through Costimulatory Molecule Blockade,” American Journal of Transplantation 15(9): 2324-2335; Balavoine, A. S., Glinoer, D., Dubucquoi, S., Wemneau, J. L. 2015, Op cit.; Chang, D. H. and Kobashigawa, J. A. 2015. “Current Diagnostic and Treatment Strategies for Cardiac Allograft Vasculopathy,” Expert Reviews of Cardiovascular Therapy 13(10): 1147-1154; Agarwal, S., Parashar, A., Kapadia, S. R., Tuzcu, E. M., Modi, D., Starling, R. C., and Oliveira, G. H. 2014. “Long-term Mortality after Cardiac Allograft Vasculopathy: Implications of Percutaneous Intervention,” Journal of the American College of Cardiology. Heart Failure 2(3): 281-288; Singh, N., Jacob, F., Rader, D. J., Vanhaecke, J., Van Cleemput, J., and De Geest, B. 2014. “Impaired Cholesterol Efflux Capacity and Vasculoprotective Function of High-density Lipoprotein in Heart Transplant Recipients,” Journal of Heart and Lung Transplantation (New York, New York) 33(5): 499-506; Skoric, B., Čikeš, M., Ljubas Maček, J., Baričević, Ž., Škorak, I., and 3 others 2014. “Cardiac Allograft Vasculopathy: Diagnosis, Therapy, and Prognosis,” Croatian Medical Journal 55(6): 562-576; Costello, J. P., Mohanakumar, T., and Nath, D. S. 2013. “Mechanisms of Chronic Cardiac Allograft Rejection,” Texas Heart Institute Journal 40(4): 395-399; Petermann Smits, D. R., Wilde, B., Kianersi Adegani. M., de Jongh, H., van Paassen, P., and Cohen Tervaert, J. W. 2013. “Metabolic Syndrome in ANCA [antineutrophil cytoplasmic antibody]-associated Vasculitis,” Rheumatology (Oxford, England) 52(1): 197-203; Prada-Delgado O., Estévez-Loureiro, R., López-Sainz, A., Gargallo-Fernandez, P., Paniagua-Martin, M. J., and 5 others 2012. “Percutaneous Coronary Interventions and Bypass Surgery in Patients with Cardiac Allograft Vasculopathy: A Single-center Experience,” Transplantation Proceedings 44(9): 2657-2659; Woodruff, T. M., Nandakumar, K. S., and Tedesco, F. 2011. “Inhibiting the C5-C5a Receptor Axis,” Molecular Immunology 48(14): 1631-1642; Raichlin, E. R., McConnell, J. P., Lerman, A., Kremers, W. K., Edwards, B. S., and 4 others 2007. “Systemic Inflammation and Metabolic Syndrome in Cardiac Allograft Vasculopathy,” Journal of Heart and Lung Transplantation (New York, New York) 26(8): 826-833).

FIG. 15A depicts the use of vascular servovalves for performing a perfusion interruption-free solid organ transplant, specifically, a heart transplant, reduced to simplest terms in that the supply and drainage vessels have been schematized as unitary. Because transplantation of the heart—the pump itself—is the most challenging procedure for off-pump heart surgery, one always distinguished from other transplant organs in a special intolerance for nonperfusion (see, for example, Fatullayev, J., Samak, M., Sabashnikov, A., Weymann, A., Mohite, P. N., and 6 others 2015, Op cit., above in this section) the one that therefore subsumes the feasibility of other organ transplants, to include the kidney, liver, gut, or lung, for example, that shown here is that of an off-pump heart transplant.

In FIG. 15A, the recipient heart is on the right and the donor on the left. FIG. 16 shows the connections between corresponding vessels in the two hearts. While these connections can be separate, collecting multiple vessels which are alike in diagnostic and therapeutic requirements for confluent flow through a confluence conduit provides not only visual clarity of the surgical field but as specified below in section 2b(1)(b)(b14), entitled Confluence Conduits: Connection of One or More Bloodlines t to Flow through a Common Channel to the Same or a Different Number of Bloodlines with Self-leveling Apportionment of Outflow, affords significant advantages, as delineated in the subsection thereof entitled Accordingly, Confluence Conduits Offer Four primary advantages:

As the process of reciprocal cross-circulation continues, blood flow is gradually transferred from the heart of the recipient to that of the donor with supportive medication to include tolerance inducing, antimicrobial, and antithrombotic released into the combined bloodstreams through the servovalve druglines shown as part number 8′ in FIG. 10A. The servovalves applied to the supply and drainage vessels of both hearts, the risks of thrombus or infection are materially reduced.

Moreover, rejection reaction sensors signal the system master control microprocessor to modulate the rate of tolerance-inducing and rejection counteracting drug delivery. The donor alone is kept on machine bypass following harvesting and positioned as closely parallel or side by side to the donor as does not interfere with the operation. The metered compound vascular bypass method provides the most direct and trauma-free method for replacing any organ.

Probably caused by “ . . . surgical trauma, ischemia to the sinus node, rejection, drug therapy, and increasing donor age,” (Lee, K. J., Jung, Y. S., Lee, C. J., Wi, J., Shin, S., and 5 others 2009 “Permanent Pacemaker for Syncope after Heart Transplantation with Bicaval Technique,” Yonsei Medical Journal (Seoul, South Korea) 50(4): 588-590), because other than the need the need for an access wound, the metered compound vascular bypass method eliminates the surgical trauma and completely eliminates the ischemia, the risk for postoperative synchronization disturbances, which nevertheless are always to be planned for in advance, should be considerably less than with a conventional technique.

Indeed, the cause is most often attributed to the biatrial as opposed to the bicaval technique (see, for example, Immohr, M. B., Boeken, U., Bruno, R. R., Sugimura, Y., Mehdiani, A., and 5 others 2022. “Optimizing Anastomoses Technique in Orthotopic Heart Transplantation: Comparison of Biatrial, Bicaval, and Modified Bicaval Technique,” Online, MDPI [Multidisciplinary Digital Publishing Institute] Journal of Cardiovascular Development and Disease (Basel, Switzerland) 9(11): 404; Rivinius R, Helmschrott M, Ruhparwar A, Erbel C, Gleissner C A, and 5 others 2017. “The Influence of Surgical Technique on Early Posttransplant Atrial Fibrillation-Comparison of Biatrial, Bicaval, and Total Orthotopic Heart Transplantation,” Online, Therapeutics and Clinical Risk Management (Albany, Auckland, New Zealand) 13:287-297; Morgan, J. A. and Edwards, N. M. 2005. “Orthotopic Cardiac Transplantation: Comparison of Outcome Using Biatrial, Bicaval, and Total Techniques,” Journal of Cardiac Surgery (Hoboken, New Jersey) 20(1): 102-106).

Using the compound vascular bypass method, there is no dissection into either the donor or the recipient heart nor are the severed parts of the two anastomosed. This would reasonably set issues such as the relative merits of the bicaval as opposed to the biatrial or total to rest. Sufficiency of function and freedom from complications not mere survival are the appropriate criteria (Weiss, E. S., Nwakanma, L. U., Russell, S. B., Conte, J. V., and Shah, A. S. 2008. “Outcomes in Bicaval versus Biatrial Techniques in Heart Transplantation: An Analysis of the UNOS [United Network for Organ Sharing] Database,” Journal of Heart and Lung Transplantation (New York, New York) 27(2): 178-183).

The biatrial technique least preferred, depending upon the degree of structural matching between the hearts of the donor and recipient, the cardiectomy and replacement technique preferred is the total orthotopic. It is believed that using the procedure set forth herein, the advantages over the total orthotopic of the bicaval technique will no longer apply. The total orthotopic technique removes the donor heart with full excision of the atria, preserving intact the structure of these, the four main pulmonary veins, venae cavae, and other vessels. From the present standpoint, this has the advantage of avoiding the direct suturing in juxtaposed, or apposed, relation of allogeneic tissues at the anastomoses—the present approach seeks to reduce if not eliminate the need for direct anastomoses.

The bicaval technique improved upon the biatrial technique in eliminating the distortion in atrial conformation when the donor heart was anastomosed to the atria of the recipient (see, for example, Grant, S. C., Khan, E. B., Faragher, M. A., Yonan, N., and Brooks, N. H. 1995, following paragraph). However, this consideration does not arise with the total orthotopic technique, and is seen as advantageous in the absence of the concept and means provided here. These techniques represent only partial transplants, the front of the defective original heart replaced with another stitched onto the original posterior portions.

To allow sufficient space to position the jackets, preserve the morphology of the heart, and prevent leakage, notably from the venae cavae, these vessels are trimmed back leaving the perivascular fat which has physiological function and transected as long as possible (see, for example, Rivinius, R., Helmschrott. M., Ruhparwar, A., Erbel. C., Gleissner, C. A., and 5 others 2017, Op cit.; Awad, M., Czer. L. S., Hou, M., Golshani. S. S., Goltche, M., and 8 others 2016. “Early Denervation and Later Reinnervation of the Heart Following Cardiac Transplantation: A Review,” Journal of the American Heart Association (Oxford, England) 5(11). pii: e004070; Dell′Aquila, A. M., Mastrobuoni, S., Bastarrika, G., Praschker, B. L., Aguero, P. A., and 3 others 2012. “Bicaval Versus Standard Technique in Orthotopic Heart Transplant: Assessment of Atrial Performance at Magnetic Resonance and Transthoracic Echocardiography,” Interactive Cardiovascular and Thoracic Surgery 14(4): 457-462; Ali, A., Robati. R., Lewis, N. P., Elshowaia. S., and Jovin, I. S. 2012. “Biatrial Masses in a Heart Transplant Patient,” Texas Heart Institute Journal 39(6): 908-909; Czer, L. S., Cohen, M. H., Gallagher, S. P., Czer. L. A., Soukiasian, H. J., and 4 others 2011. “Exercise Performance Comparison of Bicaval and Biatrial Orthotopic Heart Transplant Recipients,” Transplantation Proceedings 43(10): 3857-3862; Davies, R. R., Russo, M. J., Morgan, J. A., Sorabella, R. A., Naka, Y., and Chen, J. M. 2010. “Standard Versus Bicaval Techniques for Orthotopic Heart Transplantation: An Analysis of the United Network for Organ Sharing Database,” Journal of Thoracic and Cardiovascular Surgery 140(3): 700-708; Locali, R. F., Matsuoka, P. K., Cherbo, T., Gabriel, E. A., and Buffolo, E. 2010. “Should Biatrial Heart Transplantation Still be Performed?: A Meta analysis-,” (in English and Portuguese), Arquivos Brasileiros de Cardiologia (Brazilian Archives of Cardiology, Sao Paulo, Brazil) 94(6): 829-840; Jacob, S. and Sellke, F. 2009. Op cit. above under section 2b(1)(a) entitled Sudden Compound Vascular Bypass, or Sudden Switched-organ Transplantation; Sun, J. P., Niu, J., Banbury, M. K., Zhou, L., Taylor, D. O., and 4 others 2007. “Influence of Different Implantation Techniques on Long-term Survival after Orthotopic Heart Transplantation: An Echocardiographic Study,” Journal of Heart and Lung Transplantation (New York, New York) 26(12): 1243-1248; Weiss, E. S., Nwakanma. L. U., Russell, S. B., Conte, J. V., and Shah, A. S. 2008, Op cit.; Dreyfus, G. 2007. “Surgical Technique of Orthotopic Heart Transplantation. 2: Bicaval ‘Total’ Approach,” in Cooper, D. K. C., Miller, L. W., and Patterson, G. A., The Transplantation and Replacement of Thoracic Organs: Present Status of Biological and Mechanical Replacement of the Heart and Lungs, chapter 25, pages 215-220, New York, New York: Springer; Ambrosi, P., Ghez, O., Riberi, A., and Metras, D. 2006. “Giant Left Atrial Thrombus in a Transplanted Heart,” European Journal of Cardiothoracic Surgery 30(1): 192; Morgan, J. A. and Edwards, N. M. 2005. “Orthotopic Cardiac Transplantation: Comparison of Outcome Using Biatrial, Bicaval, and Total Techniques,” Journal of Cardiac Surgery 20(1): 102-106; Tsilimingas. N. B. 2005. “Orthotopic Cardiac Transplantation: Modification of Bicaval Anastomosis,” Annals of Thoracic Surgery 80(2): 789; Kitamura, S. 2004. “Modification of Bicaval Anastomosis for Orthotopic Cardiac Transplantation,” Annals of Thoracic Surgery 78(3): 1130; Tsilimingas, N. B. 2003. “Modification of Bicaval Anastomosis: An Alternative Technique for Orthotopic Cardiac Transplantation,” Annals of Thoracic Surgery 75(4): 1333-1334; Kitamura, S., Nakatani. T., Bando, K., Sasako, Y., Kobayashi, J., and Yagihara. T. 2001. “Modification of Bicaval Anastomosis Technique for Orthotopic Heart Transplantation,” Annals of Thoracic Surgery 72(4): 1405-1406; Riberi. A., Ambrosi, P., Habib, G., Kreitmann, B., Yao. J. G., and 3 others 2001. “Systemic Embolism: A Serious Complication after Cardiac Transplantation Avoidable by Bicaval Technique,” European Journal of Cardiothoracic Surgery 19(3): 307-312; Metras, D., Kreitmann, B., Riberi, A., Habib, G., Ambrosi, P., and 3 others 1997. “A Comparative Study of Standard and Total Orthotopic Cardiac Transplantation,” (in French with abstract at Pubmed), Archives des Maladies du Coeur et des Vaisseaux (Diseases of the Heart and Vessels) 90(1): 27-34; Grande, A. M., Pozzoli, M., Traversi. E., Martinelli, L., Minzioni, G., and 3 others 1996. “Orthotopic Heart Transplantation with Bicaval Anastomosis,” Texas Heart Institute Journal 23(4): 310-311; Grant, S. C., Khan, E. B., Faragher, M. A., Yonan, N., and Brooks, N. H. 1995. “Atrial Arrhythmias and Pacing after Orthotopic Heart Transplantation: Bicaval versus Standard Atrial Anastomosis,” British Heart Journal 74(2): 149-153; Blanche, C., Czer, L. S., Valenza, M., and Trento, A. 1994. “Alternative Technique for Orthotopic Heart Transplantation,” Annals of Thoracic Surgery 57(3): 765-767; Blanche, C., Valenza, M., Czer, L. S., Barath, P., Admon, D., and 5 others 1994. “Orthotopic Heart Transplantation with Bicaval and Pulmonary Venous Anastomoses,” Annals of Thoracic Surgery 58(5): 1505-1509; Blanche, C., Valenza, M., Aleksic. I., Czer, L. S., and Trento, A. 1994. “Technical Considerations of a New Technique for Orthotopic Heart Transplantation. Total Excision of Recipient's Atria with Bicaval and Pulmonary Venous Anastomoses,” Journal of Cardiovascular Surgery (Turin) 35(4): 283-287; Dreyfus, G., Jebara, V., Mihaileanu, S., and Carpentier, A. F. 1991. “Total Orthotopic Heart Transplantation: An Alternative to the Standard Technique,” Annals of Thoracic Surgery 52(5): 1181-1184), which does not involve resection through the posterior right atrial wall, with the donor heart implanted orthotopically.

If, as preferred, the jackets are to replace anastomoses, the vascular stumps of the recipient, to include the ascending aorta, pulmonary trunk, or main pulmonary artery, or their right and left branches, and pulmonary veins, and venae cavae, are transected so that these will extend out of the jackets to be filled with a nontoxic and nonallergenic monomer or residual monomer and antibiotic-releasing bone cement or an acrylate-based dental cement, and sutured shut. The absolute amount and substantial isolation of the cement should preclude the development of adverse side effects. The avoidance of direct donor-recipient anastomoses liberalizes the matching in size of the vessels respective of each and bypasses the interfaces where the immunologically mismatched tissues are in direct contact (see, for example, Kanter, K. R., Mahle, W. T., Vincent, R. N., and Berg, A. M. 2011, Op cit.; Li, P., Dong, N., Zhao, Y., and Gao, S. 2016 Op cit.).

The universal lack of sufficient donor hearts has forced increased tolerance for donor-recipient matching as to the relative size of the donor heart (see, for example, Jeevanandam, V., Mather, P., Furukawa, S., Todd, B., Regillo, T., Bove, A. A., McClurken, J., and Addonizio, V. P. 1994. “Adult Orthotopic Heart Transplantation Using Undersized Pediatric Donor Hearts. Technique and Postoperative Management,” Circulation 90(5 Part 2): 1174-1177; Reichart, B. 1992. “Size Matching in Heart Transplantation,” Journal of Heart and Lung Transplantation (New York, New York) 11(4 Part 2): S199-S202).

Another set of problems resolved by allowing the blood to flow through the jackets and mainlines rather than anastomosing the stumps of donor and recipient is the liberalization in juxtaposing, that is, bringing the cut ends of the donor and recipient stumps into juxtaposed relation, either when transplantation can be avoided through the Jatene arterial switch procedure, or substantial reconstruction, or when transplantation is resorted to in order to correct anomalies of orientation that are irreparable where there is an urgent need for a heart transplant. Provided the left atrium is completely detached from the thoracic wall, an additional degree of movement allowed when piping rather than direct anastomosis is employed is some liberalization in the exact rotational disposition of the donor heart, significant when the thorax is malformed, as presents with numerous congenital conditions.

While unlikely, anastomosing some while bypassing other vessels must remain a clinical judgement based upon the specific condition of the donor heart and any anatomical peculiarity of the recipient. For example, unless the need for a heart transplant is not exigent so that time permits reconstruction of the anterior thoracic wall, the heart in a patient with pectus excavatum may be considerably displaced and even rotated. If severe, the condition may have caused mitral valve prolapse, and therewith, the cardiopulmonary dysfunction that led to the need for a heart or heart and lung transplant.

Several primary conditions such as mental illness and the malfunctioning of other major organs, primarily, the kidneys and liver, scleroderma, and the CREST [calcinosis, Raynaud's phenomenon, esophageal dysmotility, sclerodactyly, and telangiectasia] syndrome can damage the heart and lungs to the degree that its replacement becomes necessary (Rotvig, C., Christensen, A. V., Juel, K., Svendsen, J. H., Jorgensen, M. B., and 5 others 2022. “The Association between Cardiac Drug Therapy and Anxiety among Cardiac Patients: Results from the National DenHeart Survey,” Online, BioMed Central Cardiovascular Disorders 22(1): 280; De Hert, M., Detraux, J. and Vancampfort, D. 2018. “The Intriguing Relationship between Coronary Heart Disease and Mental Disorders,” Online, Dialogues in Clinical Neuroscience 20(1): 31-40).

The durability of a replacement heart requires that the cause or causes which affected the native heart be mollified if not corrected. For spinal deformities, for example, this requires the placement of a somewhat smaller heart, or some readjustment in the positioning of the graft organ, or some reconstruction to minimize encroachment upon the new heart, or some combination of these. Impairment of the native heart responsive to primary mental problems requires that the psychological symptoms be ameliorated.

The abnormal mechanical forces which a severe thoracic malformity such as heterotaxy (heterotaxia, situs ambiguous, situs ambiguus, isomerism), Scheuermann's kyphosis, scoliosis, kyphoscoliosis, kyphoscoliotic Ehlers-Danlos Syndrome, straight back syndrome (thoracic lordosis), Parkinson's disease, or camptocormia (bent spine syndrome) places on the heart can also result in cardiac damage (see, for example, Mori, S., Anderson, R. H., Nishii, T., Matsumoto, K., and Loomba, R. S. 2017. “Isomerism in the Setting of the So-called “Heterotaxy”: The Usefulness of Computed Tomographic Analysis,” Annals of Pediatric Cardiology 10(2): 175-186; Hasegawa, K., Takaya, T., Mori, S., Ito, T., Fujiwara, S., and 5 others 2016. “Compression of the Right Ventricular Outflow Tract Due to Straight Back Syndrome Clarified by Low-dose Dual-source Computed Tomography,” Internal Medicine (Tokyo, Japan) 55(22): 3279-3283; Landis, B. J., Cooper, D. S., and Hinton, R. B. 2016. “CHD [coronary heart disease] Associated with Syndromic Diagnoses: Peri-operative Risk Factors and Early Outcomes,” Cardiology in the Young (Cambridge, England) 26(1): 30-52; McGovern, E., Kelleher, E., Potts, J. E., O'Brien, J., Walsh, K., Nolke, L., and McMahon, C. J. 2016. “Predictors of Poor Outcome among Children with Heterotaxy Syndrome: A Retrospective Review,” Open Heart 3(2): e000328; Harden, B., Tian, X., Giese, R., Nakhleh, N., Kureshi, S., and 10 others 2014. Increased Postoperative Respiratory Complications in Heterotaxy Congenital Heart Disease Patients with Respiratory Ciliary Dysfunction,” Journal of Thoracic and Cardiovascular Surgery 147(4): 1291-1298; Gold, P. M., Albright, B., Anani, S., and Toner, H. 2013. “Straight Back Syndrome: Positive Response to Spinal Manipulation and Adjunctive Therapy-A Case Report,” Journal of the Canadian Chiropractic Association 57(2): 143-149; Jacobs, J. P., Pasquali, S. K., Morales, D. L., Jacobs, M. L., Mavroudis, C., and 5 others 2011. “Heterotaxy: Lessons Learned about Patterns of Practice and Outcomes from the Congenital Heart Surgery Database of the Society of Thoracic Surgeons,” World Journal for Pediatric and Congenital Heart Surgery 2(2): 278-286; Teupe, C. H. and Groenefeld, G. C. 2011. “Platypnea-orthodeoxia Due to Osteoporosis and Severe Kyphosis: A Rare Cause for Dyspnea and Hypoxemia,” Heart International 6(2): e13; Jacobs, J. P., Anderson, R. H., Weinberg, P. M., Walters, H. L. 3rd, Tchervenkov, C. I., and 11 others 2007. “The Nomenclature, Definition and Classification of Cardiac Structures in the Setting of Heterotaxy,” Cardiology in the Young (Cambridge, England) 17 Supplement 2:1-28; Arlet, V., Odent, T., and Aebi, M. 2003. Congenital Scoliosis, “European Spine Journal 12:456-463; Coran, D. L., Rodgers, W. B., Keane, J. F., Hall, J. E., and Emans, J. B. 1999. “Spinal Fusion in Patients with Congenital Heart Disease. Predictors of Outcome,” Clinical Orthopaedics and Related Research(364): 99-107; Ansari, A. 1985. “The “Straight Back” Syndrome: Current Perspective More Often Associated with Valvular Heart Disease than Pseudoheart Disease: A Prospective Clinical, Electrocardiogramtgenographic, and Echocardiographic Study of 50 Patients,” Clinical Cardiology 8(5): 290-305; Izar G. 1954. “Kyphosis and the Heart,” (in Italian), Minerva Medica 45(77): 711-714; Finley, F. G. 1921. “Spinal Deformity as a Cause of Cardiac Hypertrophy and Dilatation,” Canadian Medical Association Journal 11(10): 719-723).

Should complete ligation or embolization nevertheless result in atrophy, then the ligation is less than complete, allowing some flow from each vessel to pass and mix with the greater volume of blood crossed over. To accomplish this with woven reinforced polyethylene terephthalate (Dacron®, Terylene®, woven Dacron® or Terylene® tubing requires cross clamping under cardiopulmonary support and affords no way to target drugs directly to the treatment site. While side-entry diversion jackets connected by tubes connecting their mainlines as a bypass to instantly switch the flow through each vessel to the other to be followed by conventional anastomosis is possible, it is preferable to leave the bypass as a permanent prosthesis with the ability to target medication to the treatment site.

As the pressure is greater in the larger vessel and abruptly drops moving past the suture line leading into the smaller vessel, anastomoses of vessels differing in luminal diameter produce constant stress on the suture line, already challenged as the interface between immunologically incompatible tissues placed in direct contact. By contrast, interpositioning a length of tubing between the two removes the abrupt pressure drop, and the jackets coupling the tubing to the vessel at either end have accessory channels to target medication directly to the jacket-tissue junctions, which feature is even more beneficial when used in conjunction with conventional anastomoses in allowing the direct targeting to the site of immunosuppressant medication, allowing a background systemic dose and the side effects it causes to be reduced.

Such distinctions in vessel caliber do not equate to or even correlate with differences in body weight or surface area (see, for example, Patel, N. D., Weiss, E., Nwakanma. L. U., Russell, S. D., Baumgartner, W. A., Shah, A. S., and Conte, J. V. 2008. “Impact of Donor-to-recipient Weight Ratio on Survival after Heart Transplantation: Analysis of the United Network for Organ Sharing Database,” Circulation 118(14 Supplement): S83-S88; Morley D I, Boigon M, Fesniak H, Brubaker P. Walter J., and 5 others 1993. “Posttransplantation Hemodynamics Exercise Function are not Affected by Body-size Matching of Donor and Recipient,” Journal of Heart and Lung Transplantation (New York, New York) 12(5): 770-778).

The suture lines, that is, not the suture itself, but rather the train of stitches, of anastomoses the sites most susceptible to thrombosis, stenosis, or stricture, aneurysm, pseudoaneurysm, infection, and the generation of stray discharges that cause arrhythmias even when autogenous, these risks are intensified when allogeneic tubular stumps are stitched together in direct contact over the incised surfaces. The immune system implemented primarily through the blood and endothelium, the transplant as a whole is attacked, but thinner, punctured, cut edges in direct contact are more vulnerable to breakdown.

This in a context already fraught with impediments (see, for example, Toldo, S., Quader, M., Salloum, F. N., Mezzaroma, E., and Abbate, A. 2016. “Targeting the Innate Immune Response to Improve Cardiac Graft Recovery after Heart Transplantation: Implications for the Donation after Cardiac Death,” Online, MDPI [Multidisciplinary Digital Publishing Institute] International Journal of Molecular Sciences (Basel, Switzerland) 17(6) pii: E958; Qin, L., Li, G., Kirkiles-Smith, N., Clark, P., Fang. C., and 6 others 2016. “Complement C5 [gene] Inhibition Reduces T Cell-Mediated Allograft Vasculopathy Caused by Both Alloantibody and Ischemia Reperfusion Injury in Humanized Mice,” American Journal of Transplantation 16(10): 2865-2876; Horiuchi. T. and Tsukamoto, H. 2016. “Complement-targeted Therapy: Development of C5- and C5a-targeted Inhibition,” Inflammation and Regeneration 36:11).

In FIG. 15A, each of the arterial and venous valves 18 are actually provided with at least one accessory channel into each of which can inserted a drugline 8 to directly pipe-target an immunosuppressant, anti-inflammatory, antimicrobial, or any other liquid medicinal substance or maintenance solution such as an occasional drop of a heparin to dispel clot. Druglines 8 have been omitted from FIG. 15A to preserve the simplicity and isolate the intended concept of the figure; however, part number 8 appears in many other drawing figures. In FIG. 15A, whereas the lower venous arm in a heart transplant has to convey only the circulation through the venae cavae and azygos vein, the line can be singular 196′. In contrast, the number of arteries that must be connected through the upper arm will be multiple, so that the arterial arm will almost always be a confluence conduit 230.

The risk of stump atrophy, which could result in jacket migration and failure, is additionally counteracted by means addressed elsewhere herein, and if necessary, the direct targeting to these blind pockets of medication through the accessory channel coursing along the bottom of the chute, which is connected to the port at the body surface, direct drug targeting on a permanent basis thus enabled from outside the body. Alternatively, the stumps are anastomosed, each to its counterpart, the jackets left in place to allow targeting drugs at the suture lines and pass fine cabled devices to each junction thereby providing a passageway for directly targeted treatment and viewing with entry at the surface port.

Before its advancement or deployment when the diversion jacket is placed, the ostium obturator is withdrawn inside the trepan tube where it blocks the way for passage of a miniature cabled device. When advanced into the substrate native lumen, the ostium obturator will continue to block passage until it is sufficiently advanced to provide the clearance beyond its distal face needed to access the vessel in the downstream direction and accommodate the steerability and size of the cabled device in relation to the size of the substrate native ductus and whether the ductus is straight of tortuous at the level sought to be entered. Passage in the upstream direction where the cabled device would pass along the upper side of the chute to be turned by the obturator and not restricted thus.

That is, with a side-entry servovalve, the ability to pass a miniature cabled device past the ostium obturator and diversion chute in the forward or downstream, direction, meaning through the space separating the ostium obturator from the far wall, depends upon the size of the valves, the extension of the diversion chute into the native lumen, and whether the cabled device can negotiate a space of this size. With adequate clearance to accommodate the steerability and size of the cabled device, passage can be through the mainline and trepan tube, or sidestem. If tortuous at the level to be reached, access is by Seldinger cutdown upstream to the vessel jacketed or further upstream to the vessel of which it is a branch (Hsu, C. C., Kwan, G. N., Evans-Bams, H., Rophael, J. A., and van Driel, M. L. 2016. “Venous Cutdown versus the Seldinger Technique for Placement of Totally Implantable Venous Access Ports,” Online, Cochrane Database of Systematic Reviews (Chichester, West Sussex, England) 2016(8): CD008942). The use of side-entry jackets offers considerable advantages in maintaining autografts regardless of the transplantation technique employed; the ability to either avoid suture lines altogether or to deliver drugs to suture lines argues for the use of side-entry diversion jackets.

Cardiac allograft vasculopathy does not spare children for whom the replacement of a congenitally malformed heart is their only hope for survival without constant impairment (see, for example, Asimacopoulos, E. P., Garbern, J. C., Gauvreau, K., Blume, E. D., Daly, K. P., and Singh, T. P. 2019. “Association of Clinical Rejection vs. Rejection on Protocol Biopsy with Cardiac Allograft Vasculopathy in Pediatric Heart Transplant Recipients,” Transplantation September 24; Glover, E. K., Jordan, N., Sheerin, N. S., and Ali, S. 2019. “Regulation of Endothelial-to-mesenchymal Transition by MicroRNAs in Chronic Allograft Dysfunction,” Transplantation 103(4): e64-e73; Kruger-Genge, A., Blocki. A., Franke. R. P., and Jung, F. 2019. “Vascular Endothelial Cell Biology: An Update,” Online, MDPI [Multidisciplinary Digital Publishing Institute] International Journal of Molecular Sciences (Basel, Switzerland) 20(18). pii: E4411; Gori, T. 2018. “Endothelial Function: A Short Guide for the Interventional Cardiologist,” Online, MDPI [Multidisciplinary Digital Publishing Institute] International Journal of Molecular Sciences (Basel, Switzerland) 19(12). pii: E3838; Penack, O. and Luft T. 2018. “Editorial: Endothelial Dysfunction during Inflammation and Alloimmunity,” Online, Frontiers in Immunology (Lausanne, Switzerland) 9:2886; Watanabe. K., Karimpour-Fard, A., Michael, A., Miyamoto, S. D., and Nakano, S. J. 2018. “Elevated Serum Vascular Endothelial Growth Factor and Development of Cardiac Allograft Vasculopathy in Children,” Journal of Heart and Lung Transplantation (New York, New York) 37(9): 1075-1082; Wang. S., Zhang, C., Wang, J., Yang, C., Xu. M., and 3 others 2017. “Endothelial Cells in Antibody-mediated Rejection of Kidney Transplantation: Pathogenesis Mechanisms and Therapeutic Implications,” Journal of Immunology Research 2017:8746303; van den Bosch, T. P., Kannegieter, N. M., Hesselink, D. A., Baan, C. C., and Rowshani. A. T. 2017. “Targeting the Monocyte-macrophage Lineage in Solid Organ Transplantation,” Online, Frontiers in Immunology (Lausanne, Switzerland) 8:153; Daly, K. P., Stack, M., Eisenga, M. F., Keane, J. F., Zurakowski, D., Blume, E. D., and Briscoe, D. M. 2017. “Vascular Endothelial Growth Factor A is Associated with the Subsequent Development of Moderate or Severe Cardiac Allograft Vasculopathy in Pediatric Heart Transplant Recipients,” Journal of Heart and Lung Transplantation (New York, New York) 36(4): 434-442; Wedel, J., Nakayama, H., Kochupurakkal, N. M., Koch, J., Klagsbrun, M., Bielenberg, D. R., and Briscoe, D. M. 2017. “The Intragraft Microenvironment as a Central Determinant of Chronic Rejection or Local Immunoregulation/Tolerance,” Current Opinion in Organ Transplantation 22(1): 55-63; Bruneau, S., Wedel. J., Fakhouri. F., Nakayama, H., Boneschansker. L., and 3 others 2016. “Translational Implications of Endothelial Cell Dysfunction in Association with Chronic Allograft Rejection,” Pediatric Nephrology 31(1): 41-51; Morozumi, K., Takeda, A., Otsuka, Y., Horike, K., Gotoh, N., and 3 others 2016. “Reviewing the Pathogenesis of antibody-mediated Rejection and Renal Graft Pathology after Kidney Transplantation,” Nephrology (Carlton, Victoria, Australia) 21 Supplement 1:4-8; Opgenoorth, M., Wagner, A., Passauer, J., Hohenstein, B., and Hugo. C. 2015. “Treatment of Antibody-mediated Rejection Including Immunoadsorption during First Year after Renal Transplantation-Clinical Results and Regulation of Endothelial Progenitor Cells,” Atherosclerosis. Supplements 18:67-73; Piotti, G., Palmisano, A., Maggiore, U., and Buzio, C. 2014. “Vascular Endothelium as a Target of Immune Response in Renal Transplant Rejection,” Online, Frontiers in Immunology (Lausanne, Switzerland) 5:505; Daly, K. P., Seifert, M. E., Chandraker, A., Zurakowski, D., Nohria, A., and 3 others 2013. “VEGF-C, VEGF-A and Related Angiogenesis Factors as Biomarkers of Allograft Vasculopathy in Cardiac Transplant Recipients,” Journal of Heart and Lung Transplantation (New York, New York) 32(1): 120-128; Sis, B. 2012. “Endothelial Molecules Decipher the Mechanisms and Functional Pathways in Antibody-mediated Rejection,” Human Immunology 73(12): 1218-1225; Feletou, M. (ed.) 2011. The Endothelium: Part 1: Multiple Functions of the Endothelial Cells—Focus on Endothelium-derived Vasoactive Mediators, San Rafael, California: Morgan and Claypool Life Sciences; Rao, D. A. and Pober, J. S. 2008. “Endothelial Injury, Alarmins, and Allograft Rejection” Critical Reviews in Immunology 28(3): 229-248; Ramzy, D., Rao, V., Brahm, J., Miriuka, S., Delgado, D., and Ross, H. J. 2005. Op cit., this section; Vos, I. H. and Briscoe, D. M. 2002. “Endothelial Injury: Cause and Effect of Alloimmune Inflammation,” Transplant Infectious Disease 4(3): 152-159; additional references to include the increased tendency for progression from atheromatous lesions to post transplantation vasculopathy provided below in section 2b, entitled Vascular Valves and Servovalves in the Diversion of Blood Flow, in the subsection 2b(1) thereof entitled The Intercorporeal Diversion of Blood Flow, subsection 2b(1)(b)(b2), entitled Prenatal Compound Vascular Bypass, or Switched-organ Transplantation of the Heart, which does not interfere with the collateral application of apheresis or the simultaneous, usually arterial, infusion or injection of agents not stored in and dispensed by the implanted system during its administration of the organ transfer from donor to recipient.

The cessation of life support upon brain death, excision, and isolation of the instant graft organ from the physiological and metabolical body unit wherein the organs and tissues had been intimately interdependent represents an additional insult delivered against the graft and other potentially transplantable organs. While harvesting is inevitable, functional replacement by such means as the placement of an artificial heart, renal and/or hepatic dialysis, total parenteral nutrition, ventilator, and the exogenous provision of hormones would better preserve the prospective graft organs.

The interdependence of the organs is well established, and transplantation of mutually dependent organs together might not only avert the need for later retransplantation of either based upon 1. Physiological interdependence, but because 2. One organ bolsters immune tolerance induction in the other, and especially if taken from the same donor, 3. Contributes to the ability of the other to avoid rejection. Diabetes, high blood pressure, and end-stage kidney disease are closely interrelated, cardiorenal, hepatorenal, cardiohepatic, and similar conjoint disorders such as of the lungs and liver in cystic fibrosis are common.

The relevance to such combined two and less frequently three organ transplants is that as with the heart, the substantial reduction in trauma and the elimination of anoxia of the graft organs pending implantation made possible by compound vascular bypass transplantation encourages a higher level of initial function and more lasting durability. Interdependence of the lungs and liver that may recommend the replacement of both include cystic fibrosis, pulmonary fibrosis, alcoholic cirrhosis, nonalcoholic cirrhosis, and alpha-1 antitrypsin deficiency (Bell, P. T., Carew. A., Fiene, A., Divithotawela. C., Stuart, K. A., and 3 others 2021. “Combined Heart-Lung-Liver Transplantation for Patients with Cystic Fibrosis: The Australian Experience,” Transplantation Proceedings 53(7): 2382-2389; Shah, L., Dube, G., Dove, L., Mclaughlin, A. C., Emond, J., and 3 others 2021. “Combined Liver-Lung-Kidney Transplant in a Patient with Cystic Fibrosis,” American Journal of Case Reports 22 (e930867) 1-5; Freischlag, K. W., Messina, J., Ezekian, B., Mulvihill, M. S., Barbas, A., and 5 others 2018. “Single-center Long-term Analysis of Combined Liver-Lung Transplant Outcomes,” Transplantation Direct 4(5): e349; Freischlag, K. W., Schroder, P. M., Ezekian, B., Cox, M. L., Mulvihill, M. S. Harwig, M. G., and Knechtle, S. J. 2018. “Improved Survival in Simultaneous Lung-Liver recipients and Candidates in the Modern Era of Lung Allocation,” Journal of Surgical Research 231:395-402; Han, J. L., Beal, E. W., Mumtaz, K., Washburn, K., and Black, S. M. 2018. “Combined Liver-Lung Transplantation: Indications, Outcomes, Current Experience and Ethical Issues,” Transplantation Reviews (Orlando, Florida) 33(2): 99-106; Ceulemans. L. J., Strypatein, S., Neyrinck, A., Verleden, S., Ruttens. D., and 8 others 2016. “Combined LiverOThoracic Transplantation: Single-center Experience with Introduction of the ‘Liver-first’ Principle,” Transplantation International 29(6): 715-726).

Due to this mutual dependency, substantially interdependent organs tend to fare better when transplanted together or the one shortly after the other, and the same is true of retransplantations, which more durable, are growing in support (see, for example, Awad, M. A., Czer, L. S. C., Emerson, D., Jordan, S., De Robertis. M. A., and 11 others 2019. “Combined Heart and Kidney Transplantation: Clinical Experience in 100 Consecutive Patients,” Journal of the American Heart Association (Oxford, England) 8(4): e010570; Rizvi, S. A., Luc, J. G. Y., Choi, J. H., Phan, K., Moncho Escriva, E., Patel. S., Massey, H. T., and Tchantchaleishvili, V. 2018. “Outcomes and Survival following Heart Retransplantation for Cardiac Allograft Failure: A Systematic Review and Meta-analysis,” Annals of Cardiothoracic Surgery 7(1): 12-18; Luc, J. G. Y., Choi, J. H., Rizvi, S. A., Phan, K., Moncho Escriva, E., and 7 others 2018. “Percutaneous Coronary Intervention versus Coronary Artery Bypass Grafting in Heart Transplant Recipients with Coronary Allograft Vasculopathy: A Systematic Review and Meta-analysis of 1,520 Patients,” Annals of Cardiothoracic Surgery 7(1): 19-30; Kubal. C. A., Pennington, C., Fridell, J., Ekser. B., Muhaylov, P., and Mangus, R. 2018. “Challenges with Intestine and Multivisceral Re-Transplantation: Importance of Timing of Re-Transplantation and Optimal Immunosuppression,” Annals of Transplantation 23:98-104; Gasteiger, S., Cardini. B., Gobel, G., Oberhuber, R., Messner, F., and 5 others 2018. “Outcomes of Pancreas Retransplantation in Patients with Pancreas Graft Failure,” British Journal of Surgery 105(13): 1816-1824; Perosa. M., Sergi, F., and Noujaim, H. 2018. “Outcomes after Pancreas Retransplantation: Is the Juice Worth the Squeeze?,” Current Opinion in Organ Transplantation 23(4): 461-466; Yang, J., Pan, X., Fu. S., and Zhao, H. 2018. “Pulmonary Retransplantation,” Journal of Thoracic Disease 10(7): 4632-4636; Serfas, J. D., Patel, P. A., and Krasuski, R. A. 2018. “Heart Transplantation and Mechanical Circulatory Support in Adults with Congenital Heart Disease,” Current Cardiology Reports 20(10): 81; Kuroda, K., Sunami, H., Matsumoto, Y., Nakajima, S., Sato, T., and 6 others 2017. “Percutaneous Coronary Intervention and Coronary Artery Bypass Grafting in Heart Transplant Recipients with Transplant Coronary Arterial Vasculopathy,” Transplantation Proceedings 49(1): 130-134; Cheng, R., Vanichsarn, C., Patel, J. K., Currier, J., Chang, D. H., and 4 others 2017. “Long-term Clinical and Angiographie Outcomes of Percutaneous Coronary Intervention with Everolimus-eluting Stents for the Treatment of Cardiac Allograft Vasculopathy,” Catheterization and Cardiovascular Interventions 90(1): 48-55; Kittleson, M. M. and Kobashigawa, J. A. 2017, Op cit.; Kon, Z. N., Bittle, G. J., Pasrija, C., Sanchez, P. G., Griffith, B. P., and Pierson, R. N. 3rd. 2017. “The Optimal Procedure for Retransplantation after Single Lung Transplantation,” Annals of Thoracic Surgery 104(1): 170-175; Kim, Y., Yeo. S. M., Kang, S. S., Park, W. Y., Jin, K., and 4 others 2017. “Long-term Clinical Outcomes of First and Second Kidney Transplantation in Patients with Biopsy-proven IgA Nephropathy,” Transplantation Proceedings 49(5): 992-996; Chan, C. M., Chim, T. M., Leung, K. C., Tong, C. H., Wong, T. F., and Leung, G. K. 2016. “Simultaneous Pancreas and Kidney Transplantation as the Standard Surgical Treatment for Diabetes Mellitus Patients with End-stage Renal Disease,” Hong Kong Medical Journal 22(1): 62-69; Turner, M. E., Addonizio, L. J., Richmond, M. E., Zuckerman, W. A., Vincent, J. A., Torres, A. J., and Collins, M. B. 2016. “Percutaneous Coronary Artery Revascularization Procedures in Pediatric Heart Transplant Recipients: A Large Single Center Experience,” Catheterization and Cardiovascular Interventions 88(5): 797-803; Graves, R. C. and Fine, R. N. 2016. “Kidney Retransplantation in Children following Rejection and Recurrent Disease,” Pediatric Nephrology (Berlin, Germany) 31(12): 2235-2247; Kittleson, M. M. 2016. “Changing Role of Heart Transplantation,” Heart Failure Clinics 12(3): 411-421; Baek, C. H., Lee, J. G., Park, J. H., Kim, H., Yang, W. S., and 5 others 2016. “The Clinical Outcomes of Second Kidney Transplantation in IgA Nephropathy: A Multicenter Retrospective Study,” Clinical Nephrology 86(2): 87-93; Goldraich, L. A., Stehlik, J., Kucheryavaya. A. Y., Edwards, L. B., and Ross, H. J. 2016. “Retransplant and Medical Therapy for Cardiac Allograft Vasculopathy: International Society for Heart and Lung Transplantation Registry Analysis,” American Journal of Transplantation 16(1): 301-309; Rudolph, E. N., Finger, E. B., Chandolias, N., Kandaswamy, R., Sutherland. D. E., and Dunn, T. B. 2015. “Outcomes of Pancreas Retransplantation,” Transplantation 99(2): 367-374; Bosmuller, C., Maglione, M., Margreiter, C., Dziodzio, T., Biebl. M., and 4 others 2015. “Successful Combined Pancreas Fourth-kidney Third and Pancreas Third-kidney Second Transplantation: A Case Report,” Transplantation Direct 1(6): e22; Aigner. C. 2015. “Retransplantation,” Current Opinion in Organ Transplantation 20(5): 521-526; Savla, J., Lin, K. Y., Pradhan, M., Ruebner, R. L., Rogers, R. S., and 6 others 2015. “Heart Retransplant Recipients Have Better Survival with Concurrent Kidney Transplant than with Heart Retransplant Alone,” Journal of the American Heart Association (Oxford, England) 4(12). pii: e002435; Clerkin, K. J., Thomas, S. S., Haythe, J., Schulze, P. C., Farr. M., and 5 others 2015. “Mechanical Circulatory Support as a Bridge to Cardiac Retransplantation: A Single Center Experience,” Journal of Heart and Lung Transplantation (New York, New York) 34(2): 161-166; Kim, H. S., Kim, J. Y., Kang, E. J., Choi, Y. S., Kim, J. I., and 6 others 2015. “Immunologie and Non-immunologie Complications of a Third Kidney Transplantation,” Korean Journal of Internal Medicine 30(5): 657-664; Conway, J., Manlhiot, C., Kirk, R., Edwards, L. B., McCrindle, B. W., and Dipchand, A. I. 2014. “Mortality and Morbidity after Retransplantation after Primary Heart Transplant in Childhood: An Analysis from the Registry of the International Society for Heart and Lung Transplantation,” Journal of Heart and Lung Transplantation (New York, New York) 33(3): 241-251; Belli, E., Leoni Moreno, J. C., Hosenpud, J., Rawal. B., Landolfo, K. 2014. “Preoperative Risk Factors Predict Survival following Cardiac Retransplantation: Analysis of the United Network for Organ Sharing Database,” Journal of Thoracic and Cardiovascular Surgery 147(6): 1972-1977; Friedland-Little, J. M., Gajarski, R. J., Yu. S., Donohue, J. E., Zamberlan, M. C., and Schumacher, K. R. 2014. “Outcomes of Third Heart Transplants in Pediatric and Young Adult Patients: Analysis of the United Network for Organ Sharing Database,” Journal of Heart and Lung Transplantation (New York, New York) 33(9): 917-923; Skorić, B., Čikeš, M., Ljubas Maček, J., Baričević, Ž., and 4 others 2014. “Cardiac Allograft Vasculopathy: Diagnosis, Therapy, and Prognosis,” Croatian Medical Journal 55(6): 562-576; Pozzi, M., d′Alessandro, C., Fernandez, F.2, Nguyen, A., Pavie, A., and 3 others 2014. “Who Gets a Second Heart? A Current Picture of Cardiac Retransplantation,” Transplantation Proceedings 46(1): 202-207; Ozker, E., Saritaş, B., Vuran, C., Yörüker, U., Balci, Ş., Sarisoy, O., and Türkoz, R. 2013. “Early Initiation of Peritoneal Dialysis after Arterial Switch Operations in Newborn Patients,” Renal Failure 35(2): 204-209; Saito, A., Novick, R. J., Kiaii. B., Mckenzie, F. N., Quantz, M., Pflugfelder, P., Fisher, G., and Chu, M. W. 2013. “Early and Late Outcomes after Cardiac Retransplantation,” Canadian Journal of Surgery 56(1): 21-26; Ingsathit, A., Kantachuvesiri. S., Rattanasiri. S., Avihingsanon, Y., Premasathian, N., and 4 others 2013. “Long-term Outcome of Kidney Retransplantation in Comparison with First Kidney Transplantation: A Report from the Thai Transplantation Registry,” Transplantation Proceedings 45(4): 1427-1430; Trevizol, A. P., David, A. I., Yamashita, E. T., Pecora, R. A., and D'Albuquerque, L. A. 2013. “Intestinal and Multivisceral Retransplantation results: Literature Review,” Transplantation Proceedings 45(3): 1133-1136; Buron, F., Thaunat, O., Demuylder-Mischler, S., Badet. L., Brunet, M., and 5 others 2013. “Pancreas Retransplantation: A Second Chance for Diabetic Patients?,” Transplantation 95(2): 347-352; Khan, M. S., Mery, C. M., Zafar, F., Adachi, I., Heinle, J. S., and 3 others 2012. “Is Mechanically Bridging Patients with a Failing Cardiac Graft to Retransplantation an Effective Therapy? Analysis of the United Network of Organ Sharing Database,” (St. Louis, Missouri) 31(11): 1192-1198; Luo, J. M., Chou, N. K., Chen, Y. S., Huang, S. C., Chi, N. H., Yu, H. Y., Ko. W. J., and Wang, S. S. 2012. “Heart Retransplantation for Pediatric Primary Allograft Failure,” Transplantation Proceedings 2012 44(4): 913- 914; Lee, M. S., Cheng, R. K., Kandzari, D. E., and Kirtane, A. J. 2012. “Long-term Outcomes of Heart Transplantation Recipients with Transplant Coronary Artery Disease Who Develop In-stent Restenosis after Percutaneous Coronary Intervention,” American Journal of Cardiology 109(12): 1729-1732; Lee, M. S., Sachdeva, R., Kim, M. H., and Sachdeva, R. 2012. “Long-term Outcomes of Percutaneous Coronary Intervention in Transplant Coronary Artery Disease in Pediatric Heart Transplant Recipients,” Journal of Invasive Cardiology 24(6): 278-281; Zimmer, R. J. and Lee, M. S. 2010. “Transplant Coronary Artery Disease,” Journal of the American College of Cardiology. Cardiovascular Interventions 3(4): 367-377; Vistarini, N., Pellegrini, C., Aiello, M., Alloni. A., Monterosso. C., and 4 others 2010. “Should We Perform Heart Retransplantation in Early Graft Failure?,” Transplant International 23(1): 47-53; Kanter, K. R., Vincent, R. N., Berg, A. M., Mahle, W. T., Forbess, J. M., and Kirshbom, P. M. 2004. “Cardiac Retransplantation in Children,” Annals of Thoracic Surgery 78(2): 644-649). Hepatopulmonary issues can also arise as to necessitate an urgent response.

While the sudden and metered switch methods are preferred, conventional transplantation is no less served by automatic intracorporeal support; the implantation of an automatic response system would reduce the incidence of fatal cardiac allograft vasculopathy whether the transplantation had been conventional or through the metered switch method. It is noteworthy that the continued support of the patient is carried out by the same system that administered the transplantation from the outset so that any noteworthy experience then can be recorded for future inclusion in options posed by the system. One advantage of an implanted automatic response system is the ability to directly pipeline-target medication to the coronaries to quell the cardiac allograft vasculopathy that develops to jeopardize late-term graft survival. Vasculopathy sensors are contemplated among the categories of sensors enumerated in section 2a, entitled Background of the Invention. General, in the subsection thereof entitled Sensors.

Often leading to death as its cause within one year following a conventional heart transplant, the avoidance, or at least the minimization if not the elimination, of cardiac allograft vasculopathy is one advantage of the tolerance induction incorporated as integral in metered switch heart transplantation and similar adverse sequelae that follow the transplantation of other solid organs. Generally taking about a year to become a serious risk, that the same implanted automatic system used to perform the transplantation can periodically target a statin and immunosuppressant such as basiliximab monoclonal antibody, for example to the transplant further suppresses the emergence of frank vasculopathy.

The immune response to a pathogen involves pathogen-associated molecular pattern recognition receptors, such as Toll-like and C-type lectin receptors, and is distinguishable from the response involved in the rejection of a graft organ by damage- or danger-associated molecular pattern biomolecules, or alarmins, which may be involved in vasculopathy. Increased heat alone signals inflammation, but not the cause or the specific type effector mechanism or mechanisms. The specification of sensors to be implanted as part of an automatic disorder response system depends upon the condition to be treated and the distinctive chemistry and cellular participants involved in the immune response and symptoms.

The potential applications for implanted automatic disorder response systems pertain to transplant recipients and dying prospective organ donors, as well as to living patients with one or more chronic diseases, but not self-limited acute diseases, wherewith it is essentially coextensive with the pathology of serious disease. The field of sensor development undergoing rapid expansion, with few exceptions, such as in section 2b(1)(b), the diverse sensor types required and operational mechanism of each exceeds in scope and level of detail that necessary here.

For patients undergoing solid organ transplantation, the implanted disorder response system must incorporate sensors to monitor rejection as well as infection in order to respond appropriately to these eventualities. At the same time, many diseases, notably the dementias, often comorbid in the elderly, require sensors to detect the presence and levels of disease-associated analytes (see, for example, Button, E. B., Boyce, G. K., Wilkinson, A., Stukas, S., Hayat, A., and 5 others 2019. “ApoA-1 Deficiency Increases Cortical Amyloid Deposition, Cerebral Amyloid Angiopathy, Cortical and Hippocampal Astrogliosis, and Amyloid-associated Astrocyte Reactivity in APP/PSI Mice,” Alzheimer's Research and Therapy 11(1): 44; Cisternas, P., Taylor, X., and Lasagna-Reeves, C. A. 2019. “The Amyloid-tau-neuroinflammation Axis in the Context of Cerebral Amyloid Angiopathy,” Online, MDPI [Multidisciplinary Digital Publishing Institute] International Journal of Molecular Sciences (Basel, Switzerland) 20(24). pii: E6319; Corraliza-Gomez, M., Sanchez, D., and Ganfornina, M. D. 2019. “Lipid-binding Proteins in Brain Health and Disease,” Online, Frontiers in Neurology 10:1152; Jackson, R. J., Rose, J., Tulloch, J., Henstridge, Cl., Smith, C., and Spires-Jones, T. L. 2019. “Clusterin Accumulates in Synapses in Alzheimer's Disease and is Increased in Apolipoprotein E4 Carriers,” Brain Communications 1(1): fcz003; You, Y., Perkins, A., Cisternas, P., Munoz, B., Taylor, X., and 6 others 2019. “Tau as a Mediator of Neurotoxicity Associated to Cerebral Amyloid Angiopathy,” Online, BioMed Central Acta Neuropathologica Communications (London, England) 7(1): 26; Wojtas, A. M., Kang. S. S., Olley, B. M., Gatherer, M., Shinohara, M., and 10 others 2017. Loss of Clusterin [apolipoprotein J] Shifts Amyloid Deposition to the Cerebrovasculature via Disruption of Perivascular Drainage Pathways.” Online, Proceedings of the National Academy of Sciences of the United States of America 114(33): E6962-E6971).

The coincidence of carotid disease and dementia comorbidity high in the elderly, the benefit in releasing various substances directly into the internal carotids through one or more accessory channels in the vascular side-entry servovalves used to temporarily, intermittently, or permanently bypass flow from the common to the internal and usually, the external carotid is compelling, especially as the adverse side effects such as diarrhea caused by resveratrol capsules would be avoided. For this purpose, the vascular side-entry jackets used can be of any type from those simplest, an example of which is shown in FIG. 16 of U.S. Pat. No. 11,759,186, entitled Ductus Side-entry Jackets and Prosthetic Disorder Response Systems to more complex vascular servovalves such as those shown herein in FIGS. 10A thru 10E, 25A, and 25B.

To function neuroprotectively, these substances, to include oxidized nicotinamide adenine dinucleotide (NADH), reduced nicotinamide adenine dinucleotide (NADH), and resveratrol, would have first to establish the as yet unproven benefit these have been claimed to provide, and metabolism in the liver, lungs, and elsewhere in the body having been circumvented, the beneficial effect would have to result through direct, or topical, application in humans.

More specifically, the advantage in directly pipeline-targeting drugs through the carotids, internal carotids, and into the brain is that the dosage level need not be limited to avert adverse side effects of these substances elsewhere in the body were these allowed to enter the general circulation. And if another site or sites, such as the kidneys, liver, lungs, or vascular disease, secondarily promote a decrease in cognitive function, then that site or those sites are directly targeted as well. Such sites can be ameliorated if not prevented from degradation that would contribute to cognitive impairment.

Having emerged is a relationship among nutrition, vascular disease to include hypertension and vascular stiffness, and cognitive impairment that presents certain parallels to, or would appear to reiterate, the restrictive and limiting consequences for normal development of inadequate circulation in the very young. However, in the young, it appears to be a want of pulsatile flow with Fontan circulation which is the cause of inadequate circulation, whereas in adults, it is increased pulsatility under heightened blood pressure that damages the cerebral microvasculature (Marek, M., Horyniecki, M., Fra̧czek, M., and Kluczewska, E. 2018. “Leukoaraiosis—New Concepts and Modern Imaging,” Online, Polish Journal of Radiology (Posnan, Poland) 83: e76-e81; Webb, A. J., Simoni, M., Mazzucco, S., Kuker, W., Schultz, U., and Rothwell, P. M. 2012. “Increased Cerebral Arterial Pulsatility in Patients with Leukoaraiosis: Arterial Stiffness Enhances Transmission of Aortic Pulsatility,” Online, Stroke 43(10): 2631-2636; Hachinski, V. C., Potter, P, and Merskey, H. 1987. “Leuko-araiosis,” Journal of the American Medical Association. Archives of Neurology 44(1): 21-23).

Unless long-term, the ascription to exercise of benefit to subjects with hypertension for reducing aortic stiffness and cerebrovascular hemodynamic pulsatility during early recovery from acute aerobic exercise appears lacking in consistency (see, for example, Lopes, S., Afreixo, V., Teixeira, M., Garcia, C., Leitao, C., and 7 others 2021. “Exercise Training Reduces Arterial Stiffness in Adults with Hypertension: A Systematic Review and Meta-analysis,” Journal of Hypertension 39(2): 214-222; Saz-Lara, A., Cavero-Redondo, I., Alvarez-Bueno, C., Notario-Pacheco, B. Ruiz-Grao-M. C., and Martines-Viscaino, V. 2021. “The Acute Effect of Exercise on Arterial Stiffness in Healthy Subjects: A Meta-analysis,” Online, Journal of Clinical Medine 10(2): 291; Lefferts, W. K., DeBlois, J. P., Receno, C. N., Barreira, T. V., Brutsaert, T. D., Carhart, R. L., an Hefferman, K. S. 2018. “Effects of Acute Aerobic Exercise on Arterial Stiffness and Cerebrovascular Pulsatility in Adults with and without Hypertension,” Journal of Hypertension 36(8): 1743-1752).

The benefit to be gained through implantation of a system devised to treat comorbid conditions in a manner coordinated to achieve optimal homeostasis is clear (see, for example, Debray, A., Bherer, L., Gagnon, C., Bosquet, L., Hay, E., and 3 others 2022. “A Cross-sectional Comparison of Arterial Stiffness and Cognitive Performances in Physically Active Late Pre- and Early Post-menopausal Females,” Online, Brain Sciences 12(7): 901; Fico, B. G., Miller, K. B., Rivera-Rivera, L. A., Corkery, A. T., Pearson, A. G., and 7 others 2022. “The Impact of Aging on the Association between Aortic Stiffness and Cerebral Pulsatility Index,” Online, Frontiers in Cardiovascular Medicine 9:821151; Freeberg, K. A., Craighead, D. H., Martens, C. R., You, Z., Chonchoi, M. and Seals, D. R. 2022. “Nicotinamide Riboside Supplementation for Treating Elevated Systolic Blood Pressure and Arterial Stiffness in Midlife and Older Adults,” Online, Frontiers in Cardiovascular Medicine 9:881703; Sang, D. S., Zhang, Q., Song, D. Tao, J., Wu, S. L., and Li, Y. J. 2022. “Association between Brachial-ankle Pulse Wave Velocity and Cardiovascular and Cerebrovascular Disease in Different Age Groups,” Online, Clinical Cardiology 45(3): 315-323; Bown, C. W., Khan, O. A., Moore, E. E., Liu, D., Pechman, K. R., and 9 others 2021. “Elevated Aortic Pulse Wave Velocity Relates to Longitudinal Gray and White Matter Changes,” Online, Arteriosclerosis, Thrombosis, and Vascular Biology 41(12): 3015-3024; Lefferts, W. K., Lefferts, E. C., Hibner, B. A., Smith, K. J., and Fernhall, B. 2021. “Impact of Acute Changes in Blood Pressure an Arterial Stiffness on Cerebral Pulsatile Haemodynamics in Young and Middle-aged Adults,” Online, Experimental Physiology 106(7): 1643-1653; Liu, Q., Fang, J., Cui, C., Dong, S., Gao, L., and 5 others 2021. “Association of Aortic Stiffness and Cognitive Decline: A Systematic Review and Meta-analysis,” Online, Frontiers in Aging Neuroscience 13:680205; Araghi, M., Shipley, M. J., Wilkinson, I. B., McEniery, C. M., Valencia-Hernandez, C. A., and 4 others 2020. “Association of Aortic Stiffness with Cognitive Decline: Whitehall II Longitudinal Cohort Study,” Online, European Journal of Epidemiology 35(9): 861-869; Suri, S. Chiesa, S. T., Zsoldos, E., Mackay, C. E. Filippini, N., and 8 others 2020. “Associations between Arterial Stiffening and Brain Structure, Perfusion, and Cognition in the Whitehall II Imaging Sub-study: A Retrospective Cohort Study,” Online, PLoS [Public Library of Science] Medicine 17(12): e1003467; Cui, C., Seikikawa, A., Kuller, L. H., Lopez, O. L., Newman, A. B., Kuipers, A. L., and Mackey, R. H. 2018. “Aortic Stiffness is Associated with Increased Risk of Incident Dementia in Older Adults,” Online, Journal of Alzheimer's Disease 66(1): 297-306; Jefferson, A. L., Cambronero, F. E., Liu, D., Moore, E. E., Neal, J. E., and 11 others 2018. “Higher Aortic Stiffness is Related to Lower Cerebral Blood Flow and Preserved Cerebrovascular Reactivity in Older Adults,” Online, Circulation 138(18): 1951-1962; Rajman, L., Chwalek, K., and Sinclair, D. A. 2018. “Therapeutic Potential of NAD [nicotinamide adenine dinucleotide]-boosting Molecules: The in Vivo Evidence,” Cell Metabolism 27(3): 529-547; Li, X, Lyu, P., Ren, Y., An, J., and Dong, Y. 2017. “Arterial Stiffness and Cognitive Impairment,” Journal of the Neurological Sciences 380:1-10; Mikael, L. R., Paiva, A. M. G., Gomes, M. M., Sousa, A. L. L, Jardim, P. C. B. V., and 4 others 2017. “Vascular Aging and Arterial Stiffness,” Online, Arquivos Brasileiros de Cardiologia [Brasilian Archives of Cardiology] in English 109(3): 253-258; Pase, M. P., Beiser, A., Himali, J. J., Tsao, C., Satizabal, C. L., and 3 others 2016. “Aortic Stiffness and the Risk of Incident Mild Cognitive Impairment and Dementia, Stroke 47(9): 2256-2261; Mitchell, G. F., van Bucher, M. A., Sigurdsson, S., Gotal, J. D., Jonsdottir, M. K., and 6 others 2011. “Arterial Stiffness, Pressure, and Function: The Age, Gene/Environment Susceptibility-Reykjavic Study,” Online, Brain 134 (Part 11): 3398-3407). The constraint of drugs delivered thus to the target, for which several methods are available, is addressed below in this section. The efficacy and potential side effects of dementia retarding substances follows.

Where the arteriosclerosed aorta is significantly stiffened, the conclusive remedy is to replace it. The relation of impaired blood flow to cognitive impairment that holds in the young due to malformity of the thoracic aorta, the inadequacy of Fontan circulation following reconstruction of a complex cardiac malformity best corrected through compound vascular bypass orthotopic heart transplantation, and the arteriosclerotic degradation seen in the old is reflected in the equivalence that except for the size, is the same device that should confer a remedy. The neurodegeneration seen in the aging is certainly not due to aortic stiffening alone; however, this is a prominent factor.

Associated with aging, declining cognitive function usually coexists with impaired cardiac sufficiency, consequent arteriosclerotic degeneration of the microvasculature responsible at least in part for cognitive impairment, diabetes, chronic obstructive pulmonary disease, urinary incontinence, and several other geriatric syndromes, in which type 2 diabetes with overweight is probably that most common (see, for example, Fernandez-Bermejo Ruiz, J., Dorado Chaparro, J., Santofimia Romero, M. J., Villanueva Molina, F. J., Del Toro Garcia, X., and 4 other 2022. “Bedtime Monitoring for Fall Detection and Prevention in Older Adults,” Online, International Journal of Environmental Research and Public Health 19(12): 7139; Kuzan, A., Krolewicz, E., Kustrzeba-Wojcicka, I., Lindner-Pawłowicz, K., and Sobieszczanska, M. 2022. “How Diabetes and Other Comorbidities of Elderly Patients and Their Treatment Inforence Levels of Glycation Products,” Online, International Journal of Environmental Research and Public Health 19(12): 7524; Thomas, C., Wurzer, L., Malle, E. Ristow, M., and Madreiter-Sokolowski, C. T. 2022. “Modulation of Reactive Oxygen Species Homeostasis as a Pleiotropic Effect of Commonly Used Drugs,” Online, Frontiers in Aging 3:905261; Vodosek Hojs, N., Beve, S., Ekart. R., and Hojs. R. 2020. “Oxidative Stress Markers in Chronic Kidney Disease with Emphasis on Diabetic Nephropathy,” Online, Antioxidants (Basel, Switzerland) 9(10): 925; Jaul, E. and Barron, J. 2017. “Age-related Diseases and Clinical and Public Health Implication for the 85 Year Old and Over Population,” Online, Frontiers in Public Health 5:535; van Beek, J. H. G. M., Kirkwood, T. B. L., and Bassingthwaighte 2016. “Understanding the Physiology of the Ageing Individual: Computational Modelling of Changes in Metabolism and Endurance,” Online, Interface Focus 6(2): 20150079; Haddad, M., Knani, I., Bouzidi. H., Berriche, O., Hammami, M., and Kerkeni, M., 2016. “Plasma Levels of Pentosidine, Carboxymethyl-Lysine, Soluble Receptor for Advanced Glycation End Products and Metabolic Syndrome: The Metformin Effect,” Online, Disease Markers 2016:6248264; Kerkeni, M. Saidi. A., Bouzidi. H., Letaief, A., Ben Yana, S. and Hammami, M. 2013. “Pentosidine as a Biomarker for Microvascular Complications in Type 2 Diabetic Patients,” Online, Diabetes and Vascular Disease Research 10(3): 239-245; Willemsen, S., Hartog, J. W., van Veldhuisen, D. J., van der Meer, P., Roze, J. F., and 5 others 2012. “The Role of Advanced Glycation End-products and Their Receptor on Outcome in Heart Failure Patients with Preserved and Reduced Ejection Fraction,” American Heart Journal 164(5): 742-749.e3).

Comorbidity in the elderly is appropriately responded to with the placement of a fully implanted automatic disorder response system configured to deal with multiple disorders in a coordinated manner. In more complex comorbid disease, assigning a separate but related axis of control to each morbidity, a hierarchical control system is programmed to individually and severally monitor morbidity-dedicated sensor implants and respond to the combination of morbidities as to achieve the optimal homeostasis across the set. Information pertaining to prosthetic disorder response systems will be found in copending application Ser. No. 17/689,880, entitled Prosthetic Disorder Response Systems.

More specifically, a fully implanted prosthetic disorder response system can treat medium and small vessel disease through the reliable release into the general circulation or targeted of medication to include statins, clopidogrel, aspirin, bempedoic acid, ezetimibe, zofenopril, enalapril, a PCSK9 inhibitors (except for inclisiran, which administered twice yearly past an initiation stage, does not justify the implantation of an automatic drug delivery system for this purpose alone), and others. Release thus will accommodate patients not likely to adhere to a prescription who require backup systemic or targeted dosing (see, for example, Coffin, C., Suerken, C. K., Bateman, J. R. Whitlow, C. T., Williams, B. J., and 11 others 2022. “Vascular and Microstructural Markers of Cognitive Pathology,” Online, Alzheimer's and Dementia Diagnosis, Assessment and Disease Monitoring (Amsterdam, North Holland, Netherlands) 14(1): e12332; Jansen M. G., Griffanti, L., Mackay, C. E., Anaturk, M. Melazzini, L., and 9 others 2022. “Association of Cerebral Small Vessel Disease Burden with Brain Structure and Cognitive and Vascular Risk Trajectories in Mid-to-late Life,” Online, Journal of Cerebral Blood Flow and Metabolism 42(4): 600-612).

Once available, drugs which will pass through the blood brain barrier-which becomes more permeable with age—for the prevention and treatment of small vessel-associated dementia can be released into the internal carotids through simple ductus side-entry jackets. “Animal studies have shown that diets rich in omega-3 polyunsaturated fatty acids reduce blood-brain barrier disruption associated with aging” (Barnes, S. Chowdhury, S., Gatto, N. M., Fraser, G. E., and Lee, G. J. 2021. “Omega-3 Fatty Acids are Associated with Blood-Brain Barrier Integrity in a Healthy Aging Population,” Online, Brain and Behavior 11(8): e2273)(see also Herrmann, M., Simstich, S., Fauler, G., Hofer, E., Fritz-Petrin, E., Herrmann, W., and Schmidt, R. 2021. “The Relationship between Plasma Free Fatty Acids, Cognitive Function, and Structural Integrity of the Brain in Middle-aged Healthy Humans,” Online, Aging (Albany, New York) 13(18): 22078-22091; Availone. R., Vitale, G., and Bertolotti, M. 2019. “Omega-3 Fatty Acids and Neurodegenerative Diseases: New Evidence in Clinical Trials,” Online, International Journal of Medical Sciences 20(17): 4256; Oulhaj, A., Jemeren, F., Refsum, H., Smith, A. D., and de Jager, C. A. 2016. “Omega-3 Fatty Acid Status Enhances the Prevention of Cognitive Decline by B Vitamins in Mild Cognitive Impairment,” Online, Journal of Alzheimer's Disease 50(2): 547-557).

Further elucidation of the genetics of dementia and the development of effective medication will eventually allow preventive measures to be instituted before the onset of the disease (see, for example, Kouhnavardi, S., Ecevitoglu, A., Dragacevic. V., Sanna, F. Arias-Sandoval. E., and 20 others 2022. “A Novel and Selective Dopamine Transporter Inhibitor(S)-MK-26, Promotes Hippocampal Synaptic Plasticity and Restores Effort-related Motivational Dysfunctions,” Online, Biomolecules 12(7): 881; Musaeus. C. S., Pedersen, J. S., Kjaer. T. W., Johannsen, P. Waldemar, G., and 4 others 2021. “Cortical Frontoparietal Network Dysfunction in CHMP 2B Frontotemporal Dementia,” Online, Frontiers in Aging Neuroscience 13:714220; Clarimon, J., Moreno-Grau, S., Cervera-Carles, L., Dols-Icardo, O., Sanchez-Juan, P., and Ruiz, A. 2020. “Genetic Architecture of Neurodegenerative Dementias, Neuropharmacology 15:168; Paulson, H. L. and Igo. I. 2012. “Genetics of Dementia,” Online, Seminars in Neurology 31(5): 449=460).

Section 2b(2)(c) below entitled Vascular Segment Prostheses: Inline Coupling Jackets and Bridging, or Tie-lines, and 2b (2)(c)(c1), entitled Inline Coupling Jackets Proper, along with FIGS. 36A 36E, and 37D addresses prosthetic replacement segments for diseased, malformed, or irreparably injured ductus, mostly vascular. Shown in FIGS. 37A, 37C, 37E, and 37F, the automatic action for the circulation noninterrupted ischemia-free placement of such a tie-line type segment is described below in section 2b(2)(c)(c2), entitled Automatic Insertion into a Ductus of an Inline Coupling Jacket-secured Bridging, or Tie-line and Its Replacement if Necessary. FIG. 36F shows a prosthetic aortic neoarch with supraaortic branches as an example of this type prosthesis.

Other major vessels such as much of the abdominal aorta with branches or any other larger vessel is also replaceable by such means. FIG. 37D shows the through- and-through stitching of the outer half-jackets which encircle the stumps of the substrate ductus used to fix the inline coupling jacket-secured tie-line prosthesis in position with clot repellent suture and if necessary, the automatic release by the automatic disorder control system of an occasional drop of a heparin through drugline 8 to prevent the accretion of clot on the suture traversing the substrate lumen. FIG. 37D shows the positioning of drugline 8 to periodically deliver a drop of an anticoagulant, typically a heparin, of which the need is signaled to the master control microprocessor by a local sensor, just upstream of the through- and-through sutures.

The implanted automatic disorder response system maintains vigilance of a need for any medicinal prepared in fluid form and is equipped to directly pipe-target it to the affected site. The direct application to cells of resveratrol has been found to accelerate the action of mitochondrial superoxide dismutase 2. [SOD2]. (Macmillian-Crow, L. A. and Cruthirds, D. L. 2001. “Manganese Superoxide Dismutase in Disease,” Free Radical Research 34(4): 325-336), and of G protein-coupled estrogen receptor 1, or G protein-coupled receptor 30 (GPR30) [GPER] likewise, the potential for therapeutic efficacy associated with these reactions, if any, warrants further investigation (Prossnitz, E. R. and Barton, M. (2014). “Estrogen Biology: New Insights into GPER [G protein-coupled estrogen receptor 1] Function and Clinical Opportunities,” Molecular and Cellular Endocrinology 389(1-2): 71-83)(see also Adav, S. S. and Sze, S. K. 2020. “Hypoxia-induced Degenerative Protein Modifications Associated with Aging and Age-associated Disorders,” Aging and Disease 11(2): 341-364; Arbo, B. D., Andre-Miral, C., Nasre-Nasser, R. G., Schimith, L. E., Santos. M. G., and 3 others 2020. “Resveratrol Derivatives as Potential Treatments for Alzheimer's and Parkinson's Disease,” Online, Frontiers in Aging Neuroscience 12:103; Cai, Q. and Jeong, Y. Y. 2020. “Mitophagy in Alzheimer's Disease and Other Age-related Neurodegenerative Diseases,” Cells 9(1): 150; Gilmour, B. C., Gudmundsrud, R., Frank, J., Hov. A., Lautrup. L., and 13 others 2020. “Targeting NAD⁺ in Translational Research to Relieve Diseases and Conditions of Metabolic Stress and Ageing,” Mechanisms of Ageing and Development 186:111208; Sheshadri, P. and Kumar, A. 2016. “Managing Odds in Stem Cells: Insights into the Role of Mitochondrial Antioxidant Enzyme MnSOD,” Free Radical Research (London, England) 50(5): 570-584; Bresciani, G., da Cruz, I. B., and Gonzalez-Gallego, J. 2015. “Manganese Superoxide Dismutase and Oxidative Stress Modulation,” Advances in Clinical Chemistry (New York, New York) 68:87-130; Li, C. and Zhou, H. M. 2011. “The Role of Manganese Superoxide Dismutase in Inflammation Defense,” Online, Enzyme Research (New York, New York) 2011:387176).

The opening abstract in Kitada, M., Xu. J., Ogura, Y., Monno, I., and Koya, D. 2020. “Manganese Superoxide Dismutase Dysfunction and the Pathogenesis of Kidney Disease,” Online, Frontiers in Physiology 11:755 furnishes a brief and clear introduction to the potential therapeutic value in manganese superoxide dismutase in controlling the release of erosive reactive oxygen species by mitochondria (see also Liu, M., Sun, X., Chen, B., Dai. R., Xi, Z., and Xu, H. 2022. “Insights into Manganese Superoxide Dismutase and Human Diseases,” Online, MDPI [Multidisciplinary Digital Publishing Institute] International Journal of Molecular Sciences (Basel, Switzerland) 23(24): 15893; Moretti, R. and Caruso, P. 2020. “Small Vessel Disease-related Dementia: An Invalid Neurovascular Coupling?,” Online, MDPI [Multidisciplinary Digital Publishing Institute] International Journal of Molecular Sciences (Basel, Switzerland) 21(3): 1095; Qin, S.-H., Lau, A. T. Y., Liang. Z.-L., Tan, H. W., Ji. Y.-C., and 3 others 2020. “Resveratrol Promotes Tumor Microvessel Growth via Endoglin and Extracellular Signal-regulated Kinase Signaling Pathway and Enhances the Anticancer Efficacy of Gemcitabine against Lung Cancer, Cancers (Basel, Switzerland) 12(4): 974; Braidy, N., Berg, J., Clement. J., Khorshidi, F., Poljak, A., and 3 others 2019. “Role of Nicotinamide Adenine Dinucleotide and Related Precursors as Therapeutic Targets for Age-Related Degenerative Diseases: Rationale, Biochemistry, Pharmacokinetics, and Outcomes,” Antioxidants and Redox Signaling (Larchmont, New York) 30(2): 251-294; Caruso, P., Signori. R., and Moretti, R. 2019. “Small Vessel Disease to Subcortical Dementia: A Dynamic Model, which Interfaces Aging, Cholinergic Dysregulation, and the Neurovascular Unit” Vascular Health and Risk Management 15:259-281; Cicero, A. F. G., Ruscica, M., and Banach, M. 2019. “Resveratrol and Cognitive Decline: A Clinician Perspective,” Archives of Medical Science 15(4): 936-943; Hosseini, L., 12, Vafaee, M. S., Mahmoudi, J., and Badalzadeh, R. 2019. “Nicotinamide Adenine Dinucleotide Emerges as a Therapeutic Target in Aging and Ischemic Conditions,” Biogerontology 20(4): 381-395; Poddar, S. K., Sifat, A. E., Haque, S., Nahid, N. H., Chowdhury, S., and Mehedi, I. 2019. “Nicotinamide Mononucleotide: Exploration of Diverse Therapeutic Applications of a Potential Molecule,” Biomolecules 9(1): 34; Shefa, U. Jeong, N. Y., Song, I. O., Chung, H.-J., Kim, D., Jung, J., and Huh, Y. 2019. “Mitophagy Links Oxidative Stress Conditions and Neurodegenerative Diseases,” Neural Regeneration Research 14(5): 749-756; Xie, X., Gao Y., Zeng. M., Wang. Y., Tao-Feng. W., Lu, Y.-B., and Zhang, W. P. 2019. “Nicotinamide Ribose Ameliorates Cognitive Impairment of Aged and Alzheimer's Disease Model Mice,” Metabolic Brain Disease 34(1): 353-366; Braidy, N., Grant. R., and Sachdev, P. S. 2018. “Nicotinamide Adenine Dinucleotide and Its Related Precursors for the Treatment of Alzheimer's Disease,” Current Opinion in Psychiatry 31(2): 160-166; Fricker, R. A., Green, E. L., Jenkins, S. I. and Griffin, S. M. 2018. “The Influence of Nicotinamide on Health and Disease in the Central Nervous System,” International Journal of Tryptophan Research 11:1178646918776658; Hou, Y, Lautrup, S., Cordonnier, S., Wang. Y. Croteau, D. L., and 8 others 2018. “NAD⁺ Supplementation Normalizes Key Alzheimer's Features and DNA Damage Responses in a New AD Mouse Model with Introduced DNA Repair Deficiency,” Online, Proceedings of the National Academy of Sciences of the United States of America 115(8): E1876-E1885; Sawda, C., Moussa, C., and Turner, R. S. 2017. “Resveratrol for Alzheimer's Disease,” Annals of the New York Academy of Sciences 1403(1): 142-149; Gong. B., Pan, Y., Vempati, P., Zhao, W., Knable, L. 2013. “Nicotinamide Riboside Restores Cognition through an Upregulation of Proliferator-activated Receptor-γ Coactivator 1α Regulated β-secretase 1 Degradation and Mitochondrial Gene Expression in Alzheimer's Mouse Models,” Neurobiology of Aging 34(6): 1581-1588; Ma, X., Sun, Z., Liu, Y., Jia, Y., Zhang, B., MD, I and Zhang, J. 2013. “Resveratrol Improves Cognition and Reduces Oxidative Stress in Rats with Vascular Dementia,” Neural Regeneration Research 8(22): 2050-2059; Demarin, V., 1, Podobnik, S. S., Storga-Tomic, D., and Kay, G. 2004. “Treatment of Alzheimer's Disease with Stabilized Oral Nicotinamide Adenine Dinucleotide: A Randomized, Double-blind Study,” Drugs under Experimental and Clinical Research 30(1): 27-33; Rainer, M., Kraxberger, E., Haushofer, M., Mucke, H. A., and Jellinger, K. A. 2000. “No Evidence for Cognitive Improvement from Oral Nicotinamide Adenine Dinucleotide (NADH) in Dementia,” Journal of Neural Transmission (Vienna, Austria) 107(12): 1475-1481).

The side effects of NAD and resveratrol as exemplary of drugs proposed for the treatment of dementia has been closely studied and at least for these two, found to be of little concern (see, for example, Braidy N. and Liu, Y. 2020. “NAD⁺ Therapy in Age-related Degenerative Disorders: A Benefit/Risk Analysis,” Experimental Gerontology 132:110831; Shaito, A., Posadino, A. M., Younes, N., Hasan, H., Halabi, S., and 6 others 2020. “Potential Adverse Effects of Resveratrol: A Literature Review,” Online, MDPI [Multidisciplinary Digital Publishing Institute] International Journal of Molecular Sciences (Basel, Switzerland) 21(6): 2084; Okabe, K., Yaku, K., Tobe, K., and Nakagawa, T. 2019. “Implications of Altered NAD Metabolism in Metabolic Disorders,” Journal of Biomedical Science 26:34; Martens, C. R., Denman, B. A., Mazzo, M. R., Armstrong, M. L., Reisdorph, M., and 3 others 2018. “Chronic Nicotinamide Riboside Supplementation is Well-tolerated and Elevates NAD⁺ in Healthy Middle-aged and Older Adults,” Nature Communications 9:1286; Ramirez-Garza, S. L., Laveriano-Santos, E. P., Marhuenda-Muñoz, M. Storniolo, C. E., Tresserra-Rimbau, A., Vallverdu-Queralt, A., and Lamuela-Raventos, R. M. 2018. “Health Effects of Resveratrol: Results from Human Intervention Trials,” Nutrients 10(12): 1892; Salehi. B., Mishra, A. P., Nigam, M. Sener, B., Kilic, M. and 4 others 2018. “Resveratrol: A Double-edged Sword in Health Benefits,” Biomedicines 6(3): 91; Berman, A. Y., Motechin, R. A., Wiesenfeld, M. Y., and Holz, M. K. 2017. “The Therapeutic Potential of Resveratrol: A Review of Clinical Trials,” Nature Partner Journals Precision Oncology 1:35).

Sensors to detect analytes diagnostic for disease and adverse reactions, and the drugs to suppress these are identified on the basis of immunological and biochemical studies. The development of engineered sensors is expedited through the study and emulation of biological sensors, or biosensors. Sensors that must be supplied with living cells can be replenished as needed through an implanted drug reservoir as would any other agent (see, for example, Zhao, Y., Sarkar, A., and Wang, X. 2020. “Peptide Nucleic Acid Based Tension Sensor for Cellular Force Imaging with Strong DNase Resistance,” Biosensors and Bioelectronics 150:111959; Blanchard, A. T. and Salaita, K. 2019. “Emerging Uses of DNA Mechanical Devices,” Science (New York, New York) 365(6458): 1080-1081; Glazier, R., Brockman, J. M., Bartle, E., Mattheyses, A. L., Destaing. O., and Salaita, K. 2019. “DNA Mechanotechnology Reveals that Integrin Receptors Apply pN [piconewton] Forces in Podosomes on Fluid Substrates,” Nature Communications 10(1): 4507; Howden, A. J. M., Hukelmann, J. L., Brenes, A., Spinelli, L., Sinclair, L. V., Lamond, A. I., and Cantrell, D. A. 2019. “Quantitative Analysis of T cell Proteomes and Environmental Sensors during T Cell Differentiation,” Nature Immunology 20(11): 1542-1554; Parvinian, B., Pathmanathan, P., Daluwatte. C., Yaghouby, F., Gray, R. A., and 3 others 2019. “Credibility Evidence for Computational Patient Models Used in the Development of Physiological Closed-loop Controlled Devices for Critical Care Medicine,” Online, Frontiers in Physiology 10:220; Ron-Harel, N., Ghergurovich, J. M., Notarangelo, G., LaFleur, M. W., Tsubosaka, Y., and 3 others 2019. “T Cell Activation Depends on Extracellular Alanine,” Cell Reports 28(12): 3011-3021.e4; Fasbender, F. and Watzl. C. 2018. “Impedance-based Analysis of Natural Killer Cell Stimulation,” (Nature Research) Scientific Reports 8(1): 4938; Piscopo, N. J., Mueller, K. P., Das. A., Hematti. P., Murphy, W. L., and 3 others 2018. “Bioengineering Solutions for Manufacturing Challenges in CAR [chimeric antigen receptor] T Cells,” Biotechnology Journal 2018 13(2); Bishu, S. 2016. “Sensing of Nutrients and Microbes in the Gut,” Current Opinion in Gastroenterology 32(2): 86-95; Eleftheriadis, T., Pissas, G., Antoniadi, G., Liakopoulos, V., Tsogka, K., Sounidaki, M., and Stefanidis, I. 2016. “Differential Effects of the Two Amino Acid Sensing Systems, the GCN2 Kinase [general control nonderepressible 2 serine/threonine-protein kinase, senses a deficiency in amino acids] and the mTOR [mammalian target of rapamycin] Complex 1, on Primary Human Alloreactive CD4⁺ T-cells,” International Journal of Molecular Medicine 37(5): 1412-1420; Liu, Y., Blanchfield, L., Ma, V. P., Andargachew, R., Galior, K., and 3 others 2016. “DNA-based Nanoparticle Tension Sensors Reveal that T-cell Receptors Transmit Defined pN [piconewton] Forces to Their Antigens for Enhanced Fidelity,” Online, Proceedings of the National Academy of Sciences of the United States of America 113(20): 5610-5615; Ramsay, G. and Cantrell, D. 2015. “Environmental and Metabolic Sensors that Control T Cell Biology,” Online, Frontiers in Immunology (Lausanne, Switzerland) 6:99; Revzin, A., Maverakis, E., and Chang, H. C. 2012. “Biosensors for Immune Cell Analysis—A Perspective,” Biomicrofluidics 6(2): 21301-2130113).

An exemplary citation of references here and in the sections below on metered compound vascular bypass, or sudden switched-organ transplantation can no more than indicate the scope of the subject (see, for example, Howard, M. C., Nauser, C. L., Farrar, C. A., Wallis, R., and Sacks, S. H. 2020. “1-Fucose Prevention of Renal Ischaemia/Reperfusion Injury in Mice,” Federation of American Societies for Experimental Biology Journal 34(1): 822-834; Chen, X., Wang. L., Deng. Y., Li, X., Li, G., and 6 others 2019. “Inhibition of Autophagy Prolongs Recipient Survival through Promoting CD8+ T Cell Apoptosis in a Rat Liver Transplantation Model,” Online, Frontiers in Immunology (Lausanne, Switzerland) 10:1356; Grafals, M. and Thurman, J. M. 2019. “The Role of Complement in Organ Transplantation,” Online, Frontiers in Immunology (Lausanne, Switzerland) 10:2380; Horwitz, J. K., Chun, N. H., and Heeger, P. S. 2019. “Complement and Transplantation: From New Mechanisms to Potential Biomarkers and Novel Treatment Strategies,” Clinics in Laboratory Medicine 39(1): 31-43; Mueller, F. B., Yang, H., Lubetzky, M., Verma, A., Lee, J. R., and 8 others 2019. “Landscape of Innate Immune System Transcriptome and Acute T Cell-mediated Rejection of Human Kidney Allografts,” Journal of Clinical Investigation Insight 4(13). pii: 128014; Ye, X., Zhou, X. J., and Zhang, H. 2019. “Autophagy in Immune-related Renal Disease,” Journal of Immunology Research 2019:5071687; Zipfel, P. F., Wiech, T., Rudnick, R., Afonso, S., Person, F., and Skerka, C. 2019. “Complement Inhibitors in Clinical Trials for Glomerular Diseases,” Online, Frontiers in Immunology (Lausanne, Switzerland) 10:2166; Lakkis, F. G. and Li. X. C. 2018. “Innate Allorecognition by Monocytic Cells and Its Role in Graft Rejection,” American Journal of Transplantation 18(2): 289-292; Nauser, C. L., Howard, M. C., Fanelli, G., Farrar, C.A.1, and Sacks, S. 2018. “Collectin-11 (CL-11) is a Major Sentinel at Epithelial Surfaces and Key Pattern Recognition Molecule in Complement-mediated Ischaemic Injury,” Online, Frontiers in Immunology (Lausanne, Switzerland) 9:2023; Ozen, S. and Batu, E. D. 2018. “Vasculitis Pathogenesis: Can We Talk about Precision Medicine?,” Online, Frontiers in Immunology (Lausanne, Switzerland) 9:1892; Nauser, C. L., Farrar, C. A., and Sacks, S. H. 2017. “Complement Recognition Pathways in Renal Transplantation,” Journal of the American Society of Nephrology 28(9): 2571-2578; Xiao. T. S. 2017. “Innate Immunity and Inflammation,” Cellular and Molecular Immunology 14(1): 1-3; Farrar, C. A., Zhou, W., and Sacks. S. H. 2016. “Role of the Lectin Complement Pathway in Kidney Transplantation,” Immunobiology 221(10): 1068-1072; Sun, H., Cheng, D., Ma. Y., Wang. H., Liang. T., and Hou, G. 2016. “Autophagy in Allografts Rejection: A New Direction?,” Biochemical and Biophysical Research Communications 471(4): 572-575; Sharma, S., Fitzgerald, K. A., Cancro, M. P., and Marshak-Rothstein, A. 2015. “Nucleic Acid-sensing Receptors: Rheostats of Autoimmunity and Autoinflammation,” Journal of Immunology (Baltimore, Maryland) 195(8): 3507-3512; Spahn, J. H., Li, W., and Kreisel, D. 2014. “Innate Immune Cells in Transplantation,” Current Opinion in Organ Transplantation 19(1): 14-19; Farrar, C. A., Kupiec-Weglinski, J. W., and Sacks, S. H. 2013. “The Innate Immune System and Transplantation,” Cold Spring Harbor Perspectives in Medicine 2013 3(10): a015479; Sacks, S. H. and Zhou, W. 2012. “The Role of Complement in the Early Immune Response to Transplantation,” Nature Reviews. Immunology 12(6): 431-442; Li, T., Chen, G., and Zhang, Z. 2011. “Roles of Toll-like Receptors Signaling in Organ Transplantation,” Zhong Non da Xue Cue Bao. Yi Xue Ban (Journal of Central South University. Medical Sciences) 36(12): 1125-1133; Sanchez-Fueyo, A. and Strom, T. B. 2011. “Immunologic Basis of Graft Rejection and Tolerance following Transplantation of Liver or Other Solid Organs,” Gastroenterology 140(1): 51-64; Janeway, C. A. Jr., Travers. P., Walport, M., and Shlomchik, M. J. 2001. “Responses to Alloantigens and Transplant Rejection,” in Immunobiology: The Immune System in Health and Disease, New York, New York: Garland Science/Taylor and Francis; Janeway, C. A. Jr., Travers, P. Walport, M. and Shlomchik, M. J. 2001. “The Complement System and Innate Immunity,” in Immunobiology, New York, New York: Garland Science/Taylor and Francis).

The progression of vasculopathy more frequent in hearts with donor-transmitted atherosclerosis, the preconditioning of metered compound vascular bypass, or sudden switched-organ transplantation and maintenance on a statin and/or other low density lipoprotein suppressants specified below in this section and immunosuppressant reduces the undesirability of such hearts when available. Whether for switched-organ orthotopic or double heart transplant, donor transmitted atherosclerotic lesions are angioplastied before the donor heart is implanted (Watanabe, T., Seguchi, O., Yanase, M., Fujita, T., Murata, Y., and 16 others 2017. “Donor-transmitted Atherosclerosis Associated with Worsening Cardiac Allograft Vasculopathy after Heart Transplantation: Serial Volumetric Intravascular Ultrasound Analysis,” Transplantation 101(6): 1310-1319; Bedanova, H., Orban, M., Tretina, M., Fila, P., Horvath, V., Krejci, J., and Nemec, P. 2016. “Monitoring of Allograft Vasculopathy by Intravascular Ultrasound One Month and One Year after Heart Transplantation: A Single Center Study,” Biomedical Papers of the Medical Faculty of the University Palacky, Olomouc, Czech Republic 160(1): 130-135)(see also, for example, Parikh, R. V., Khush, K., Pargaonkar, V. S., Luikart, H., Grimm, D., and 6 others 2019. “Association of Endothelin-1 with Accelerated Cardiac Allograft Vasculopathy and Late Mortality following Heart Transplantation,” Journal of Cardiac Failure 25(2): 97-104; Asleh, R., Briasoulis, A., Pereira, N. L., Boilson, B. A., Edwards. B. S., and 5 others 2018. “Timing of 3-hydroxy-3-methylglutaryl-coenzyme A Reductase Inhibitor Initiation and Allograft Vasculopathy Progression and Outcomes in Heart Transplant Recipients,” European Society of Cardiology Heart Failure 5(6): 1118-1129; Flore, V., Brown, A. J., Pettit, S., West, N. E. J., Lewis, C., Parameshwar, J., and Hoole, S. P. 2018, Op cit., above in this section; Lee, M. S., Tadwalkar, R. V., Fearon, W. F., Kirtane, A. J., Patel, A. J., and 3 others 2018. “Cardiac Allograft Vasculopathy: A Review,” Catheterization and Cardiovascular Interventions 92(7): E527-E536; Zanchin, C1, Yamaji, K., Rogge. C., Lesche, D., Zanchin, T., and 5 others 2018. “Progression of Cardiac Allograft Vasculopathy Assessed by Serial Three-vessel Quantitative Coronary Angiography,” Online, PLoS [Public Library of Science] One 13(8): e0202950; Clerkin, K. J., Ali, Z. A., and Mancini, D. M. 2017. “New Developments for the Detection and Treatment of Cardiac Vasculopathy,” Current Opinion in Cardiology February 15; Hunt, S. A. 2017. “Cardiac Allograft Vasculopathy: It Really has Changed over Time,” Journal of the American College of Cardiology. Heart Failure 5(12): 902-903; Chih, S., Chong, A. Y., Mielniczuk, L. M., Bhatt, D. L., and Beanlands. R. S. 2016. “Allograft Vasculopathy: The Achilles' Heel of Heart Transplantation,” Journal of the American College of Cardiology 68(1): 80-91; Eisen, H. J. 2016. “Complications after Cardiac Transplantation,” Chapter 19, pages 314-328 in Domanski, M. J., Mehra, M. R., and Pfeffer, M. A. (eds.), Oxford Textbook of Advanced Heart Failure and Cardiac Transplantation, Oxford, England: Oxford University Press; Okada, K., Fearon, W. F., Luikart, H., Kitahara, H., Otagiri, K., and 8 others 2016. “Attenuated-signal Plaque Progression Predicts Long-term Mortality after Heart Transplantation: IVUS [intravascular ultrasound] Assessment of Cardiac Allograft Vasculopathy. Journal of the American College of Cardiology 68(4): 382-392; Raissadati, A., Pihkala, J., Jahnukainen, T., Jokinen, E., Jalanko, H., and Sairanen, H. 2016. “Late Outcome after Paediatric Heart Transplantation in Finland” Interactive Cardiovascular and Thoracic Surgery 23(1): 18-25; Badano, L. P., Miglioranza, M. H., Edvardsen, T., Colafranceschi, A. S., Muraru, D., and 7 others 2015. “European Association of Cardiovascular Imaging/Cardiovascular Imaging Department of the Brazilian Society of Cardiology Recommendations for the Use of Cardiac Imaging to Assess and Follow Patients after Heart Transplantation,” European Heart Journal Cardiovascular Imaging 16(9): 919-948; Wang, R., Moura, L. A., Lopes, S. V., Costa, F. D., Souza Filho, N. F., and 3 others 2015. “Reduced Progression of Cardiac Allograft Vasculopathy with Routine Use of Induction Therapy with Basiliximab,” Arquivos Brasileiros de Cardiologia 105(2): 176-183; Agarwal. S., Parashar, A., Kapadia, S. R., Tuzcu, E. M., Modi, D., Starling, R. C., and Oliveira, G. H. 2014, Op cit., this section; Costello, J. P., Mohanakumar, T., and Nath, D. S. 2013. “Mechanisms of Chronic Cardiac Allograft Rejection,” Texas Heart Institute Journal 40(4): 395-399; Segura, A. M. and Buja, L. M. 2013. “Cardiac Allograft Vasculopathy: A Complex Multifactorial Sequela of Heart Transplantation” Texas Heart Institute Journal 40(4): 400-402; Yamasaki, M., Sakurai. R., Hirohata, A., Honda, Y., Bonneau, H. N., and 3 others 2011. “Impact of Donor- transmitted Atherosclerosis on Early Cardiac Allograft Vasculopathy: New Findings by Three-dimensional Intravascular Ultrasound Analysis,” Transplantation 91(12): 1406-1411; Suzuki, J., Isobe, M., Morishita. R., and Nagai, R. 2010. “Characteristics of Chronic Rejection in Heart Transplantation: Important Elements of Pathogenesis and Future Treatments,” Circulation Journal 74(2): 233-239; Raichlin, E., Bae, J. H., Kushwaha, S. S., Lennon, R. J., Prasad, A., Rihal, C. S., and Lerman, A. 2009. “Inflammatory Burden of Cardiac Allograft Coronary Atherosclerotic Plaque is Associated with Early Recurrent Cellular Rejection and Predicts a Higher Risk of Vasculopathy Progression,” Journal of the American College of Cardiology 53(15): 1279-1286; Ramzy, D., Rao. V., Brahm, J., Miriuka, S., Delgado, D., and Ross, H. J. 2005. Op cit., section 2a).

That the damage done to a graft organ is directly proportional to ischemia during preservation and cardiopulmonary bypass times with cardioplegia, and cross-clamping, and harm to the recipient often done as a result of the need for general rather than regional anesthesia is universally acknowledged: “ . . . it is now important to be familiar with multiple new technical challenges associated with the surgical techniques of heart transplantation with an ultimate goal in reducing donor heart ischemic time, recipient cardiopulmonary bypass time and post-operative complications,” (Cheng, A. and Slaughter, M. S. 2014. “Heart Transplantation,” Journal of Thoracic Disease 6(8): 1105-1109. (Op cit.)(see also Singh D. and Taylor, D. O. 2015. “Advances in the Understanding and Management of Heart Transplantation,” F1000 [faculty of 1000] Prime Reports 7:52: Rodrigues, W., Carr, M., Ridout, D., Carter, K., Hulme, S. L., and 5 others 2011. “Total Donor Ischemic Time: Relationship to Early Hemodynamics and Intensive Care Morbidity in Pediatric Cardiac Transplant Recipients,” Pediatric Critical Care Medicine 12(6): 660-666).

That compound vascular bypass organ transplantation in a deceased donor sustainment center as described above in this section under the heading Deceased Donor Sustainment Centers so that the recipient and sustained donor are compresent completely eliminates ischemia is of the utmost importance for graft durability. Ischemia preceding and during the transplantation process is suspected to contribute to long-term cardiac allograft vasculopathy and/or cancer.

Usually progressing slowly until final failure, cardiac allograft vasculopathy can cause arrhythmias along the way or proceed directly to termination through sudden cardiac death, and insidiously, the heart having been denervated when harvested, the want of innervation conceals the signs of cardiac allograft vasculopathy (Skoric. B., Čikeš, M., Ljubas Maček, J., Baričević, Ž., Škorak, I., and 3 others 2014. “Cardiac Allograft Vasculopathy: Diagnosis, Therapy, and Prognosis,” Online, Croatian Medical Journal (Zagreb, Croatia) 55(6): 562-576). (see also van den Hoogen, P., Huibers, M. M. H., van den Dolder, F. W., de Weger R. Siera-de Koning E, and 7 others 2022. “Elevated Plasma Immunoglobulin Levels Prior to Heart Transplantation are Associated with Poor Post-transplantation Survival, Online, MDPI [Multidisciplinary Digital Publishing Institute] Biology (Basel, Switzerland) 12(1): 61).

As indicated above in this section, if diagnosed, secondarily treating the vasculopathy with eluting stents, angioplasty, and coronary artery bypass grafting affords no more than a temporary improvement (see, for example, Hirose, M., Narita, J., Hashimoto, K. Ishii, R., Ishida, H., and Ozono, K. 2023. “Use of Drug-coated Balloon Instead of Drug-eluting Stent for Pediatric Cardiac Allograft Vasculopathy,” Online, Annals of Pediatric Cardiology (Mumbai [Bombay] India) 16(1): 45-47; Sharma. V., Agarwal. S., Grover. T., Malhotra, A., Kumar, H., and 5 others 2023. “Coronary Allograft Vasculopathy in Post-heart Transplant Patients: Pathogenesis and Role of Cardiac Computed Tomography in Diagnosis—A Comprehensive Review,” Annals of Medicine and Surgery (London, England) 85(7): 3531-3537; Pober, J. S., Chih, S., Kobashigawa, J., Madsen, J. C., and Tellides, G. 2021. “Cardiac Allograft Vasculopathy: Current Review and Future Research Directions,” Online, Cardiovascular Research (London, England) 117(13): 2624-2638; Pyka. L., Hawranek. M., Szygula-Jurkiewicz, B., Desperak, P., Szczurek, W., and 6 others 2020. “Everolimus-eluting Second-generation Stents for Treatment of De Novo Lesions in Patients with Cardiac Allograft Vasculopathy,” Online, Annals of Transplantation (Smithtown, New York) 25: e921266; Cheng, R., Vanichsam, C., Patel, J. K., Currier, J., Chang, D. H., and 4 others 2017, Op cit., above in this section); Skoric, B., Čikeš, M., Ljubas Maček, J., Baričević, Ž., Škorak, I., and 3 others 2014. “Cardiac Allograft Vasculopathy: Diagnosis, Therapy, and Prognosis,” Online, Croatian Medical Journal (Zagreb, Croatia) 55(6): 562-576).

The propensity for heart and lung recipients to develop cancer is addressed below in section 2b(1), entitled The Intercorporeal Diversion of Blood Flow.

Numerous conditions can affect an electrocardiogram, to include chronic kidney disease, sarcoidosis, hypothyroidism, diabetes, heavy smoking, whether the affected heart is a transplant, obesity, and weight loss, for which the prescription-program of the master control microcontroller must be adjusted (see, for example, Fernandes, F. M., Silva, E. P., Martins. R. R., and Oliveira, A. G. 2018. “QTc [corrected start of electrocardiogram Q to end of T wave duration] Interval Prolongation in Critically Ill Patients: Prevalence, Risk Factors, and Associated Medications,” Online, PLoS [Public Library of Science] One 13(6): e0199028; Kobayashi, S., Nagao, M., Asai, A., Fukuda, I., Oikawa, S., and Sugihara, H. 2018. “Severity and Multiplicity of Microvascular Complications are Associated with QT Interval Prolongation in Patients with Type 2 Diabetes,” Journal of Diabetes Investigation (Tokyo, Japan) 9(4): 946-951; Omran, J., Bostick, B. P., Chan, A. K., and Alpert, M. A. 2018. “Obesity and Ventricular Repolarization: a Comprehensive Review,” Progress in Cardiovascular Diseases 61(2): 124-135; Zolezzi, M. and Cheung. L. 2018. A Literature-based Algorithm for the Assessment, Management, and Monitoring of Drug-induced QTc Prolongation in the Psychiatric Population, “Neuropsychiatric Disease and Treatment 15:105-114; Etchegoyen, C. V., Keller, G. A., Mrad. S., Cheng. S., and Di Girolamo, G. 2017. “Drug-induced QT Interval Prolongation in the Intensive Care Unit,” Current Clinical Pharmacology 12(4): 210-222; Sertbas, Y., Ozdemir, A., Sertbas, M., Dayan, A., Sancak, S., and Uyan, C. 2017. “The Effect of Glucose Variability on QTe Duration and Dispersion in Patients with Type 2 Diabetes Mellitus,” Pakistan Journal of Medical Sciences 33(1): 22-26; Okutucu, S., Karakulak, U. N., Aksoy, H., Sabanoglu, C., Hekimsoy, V., and 7 others 2016. “Prolonged Tp-e Interval and Tp-e/QT [electrocardiogra wave landmarks] Correlates Well with Modified Rodnan Skin Severity Score in Patients with Systemic Sclerosis,” Cardiology Journal (Gdansk, Poland) 23(3): 242-249; Omran, J., Firwana, B., Koerber. S., Bostick, B., and Alpert, M. A. 2016. “Effect of Obesity and Weight Loss on Ventricular Repolarization: A Systematic Review and Meta-analysis,” Obesity Reviews 17(6): 520-530; Ucsular. F., Karadeniz, G., Karadeniz, C., Yalruz, E., and Demir, M. 2016. “Evaluation of Tpeak-Tend Interval and Tpeak-end/QT Ratio in Patients with Sarcoidosis,” Sarcoidosis, Vasculitis, and Diffuse Lung Diseases (Milan, Italy) 33(2): 151-156; Alpert, M. A., Nusair, M. B., Mukerji, R., Omran, J., Mehra. A., and 3 others 2015. “Effect of Weight Loss on Ventricular Repolarization in Normotensive Severely Obese Patients with and without Heart Failure,” American Journal of Medical Sciences 349(1): 17-23; Ari, H., Ari, S., Coşar, S., Celiloğlu, N., Aktaş. 1., and 4 others 2015. “The Effect of Varenicline on Tp-e Interval. Tp-e/QT Ratio and Tp-e/QTe Ratio in Healthy Smokers and Nonsmokers,” Cardiology Journal (Gdańsk, Poland) 22(5): 551-556; Hung. C. S., Tseng, P. H., Tu. C. H., Chen, C. C., Liao, W. C., and 7 others 2015. “Nonalcoholic Fatty Liver Disease is Associated with QT Prolongation in the General Population,” Journal of the American Heart Association (Oxford, England) 4(7). pii: e001820; Tisdale, J. E., Jaynes, H. A., Kingery, J. R., Overholser, B. R., Mourad, N. A., Trujillo, T. N., and Kovacs, R. J. 2014. “Effectiveness of a Clinical Decision Support System for Reducing the Risk of QT Interval Prolongation in Hospitalized Patients,” Circulation. Cardiovascular Quality and Outcomes 7(3): 381-390; Nielsen, J., Graff. C., Kanters, J. K., Toft, E., Taylor, D., and Meyer, J. M. 2011. “Assessing QT Interval Prolongation and Its Associated Risks with Antipsychotics,” Central Nervous System Drugs (Auckland, New Zealand) 25(6): 473-490; Christensen, T. F., Tamow, L., Randlov, J., Kristensen, L. E., Struijk, J. J., Eldrup, E., and Hejlesen, O. K. 2010. “QT Interval Prolongation during Spontaneous Episodes of Hypoglycaemia in Type 1 Diabetes: The Impact of Heart Rate Correction,” Diabetologia 53(9): 2036-2041).

If either heart experienced a sudden arrest or ventricular fibrillation not reversible for more than a few seconds, more likely in patients with chronic kidney disease on dialysis, then rather than permitting the functioning heart to pull or push the setpoint adjusted proportional volume of venous return blood based upon the absolute stroke volumes of either heart through the abnormally functioning heart, risking malfunction of the functioning heart, as well as the cessation of function of the lungs and brain, and pulseless electrical activity of asystole if not the cessation of circulation, the control microprocessor adjusts the valves to pass only so much blood through the nonfunctioning heart to supply it with sufficient oxygen to avert a sudden arrest and death-associated ‘cytokine storm’ (cytokine, or polypeptide mediator, release syndrome, or severe reaction systemic inflammatory response).

This continues until a detailed analysis and reversal can be conducted in the clinic (see, for example, Burak, C., Baysal, E., Süleymanoğlu, M., Yayla, C., Cay, S., and Kervan, Ü. 2019. “Evaluation of Myocardial Dispersion of Repolarization in Patients with Heart Transplantation,” Turkish Journal of Medical Sciences 49(1): 212-216; Gando, S. and Wada, T. 2019. “Disseminated Intravascular Coagulation in Cardiac Arrest and Resuscitation,” Journal of Thrombosis and Haemostasis 17(8): 1205-1216; Komblith, L. Z., Moore, H. B., and Cohen, M. J. 2019. “Trauma-induced Coagulopathy: The Past, Present, and Future,” Journal of Thrombosis and Haemostasis 17(6): 852-862; Lother, A., Benk, C., Staudacher, D. L., Supady, A., Bode, C., Wengenmayer, T., and Duerschmied, D. 2019. “Cytokine Adsorption in Critically Ill Patients Requiring ECMO Support,” Online, Frontiers in Cardiovascular Medicine 6:71; Mai, N., Miller-Rhodes, K., Knowlden, S., and Halterman, M. W. 2019. “The Post-cardiac Arrest Syndrome: A Case for Lung-Brain Coupling and Opportunities for Neuroprotection,” Journal of Cerebral Blood Flow and Metabolism 39(6): 939-958; Sattler, K., El-Battrawy, I., Zhao, Z., Schrottenberg. C., Yucel, G., and 12 others 2019. “Serum of Patients with Acute Myocardial Infarction Prevents Inflammation in iPSC [induced pluripotent stem cell]-cardiomyocytes,” (Nature Research) Scientific Reports 9(1): 5651; Trummer, G., Benk, C., and Beyersdorf, F. 2019. “Controlled Automated Reperfusion of the Whole Body after Cardiac Arrest,” Journal of Thoracic Disease (Hong Kong, China) 11 (Supplement 10): S1464-S1470; Buchtele, N., Schober, A., Schoergenhofer, C., Spiel, A. O., Mauracher, L., and 4 others 2018. “Added Value of the DIC [disseminated intravascular coagulation] Score and of D-dimer to Predict Outcome after Successfully Resuscitated Out-of-hospital Cardiac Arrest,” European Journal of Internal Medicine 57:44-48; Gall, L. S. and Davenport, R. A. 2018. “Fibrinolysis and Antifibrinolytic Treatment in the Trauma Patient,” Current Opinion in Anesthesiology 31(2): 227-233; Jou, C., Shah, R., Figueroa, A., and Patel, J. K. 2018. “The Role of Inflammatory Cytokines in Cardiac Arrest,” Journal of Intensive Care Medicine 10:885066618817518; Kollu, K., Altintepe, L., Duran, C., Topal, M., and Ecirli, S. 2018. “The Assessment of P-wave Dispersion and Myocardial Repolarization Parameters in Patients with Chronic Kidney Disease,” Renal Failure 40(1): 1-7; Madurska, M. J., Sachse, K. A., Jansen, J. O., Rasmussen, T. E., and Morrison, J. J. 2018. “Fibrinolysis in Trauma: A Review,” European Journal of Trauma and Emergency Surgery 44(1): 35-44; Parish, D. C., Goyal, H., and Dane, F. C. 2018. “Mechanism of Death: There's More to It than Sudden Cardiac Arrest,” Journal of Thoracic Disease (Hong Kong, China) 10(5): 3081-3087; Shimabukuro- Vornhagen, A., Godel, P., Subklewe, M., Stemmler, H. J., SchloBer, H. A., and 4 others 2018. “Cytokine Release Syndrome,” Journal for Immunotherapy of Cancer 6(1): 56; Sun, M., Ishii, R., Okumura, K., Krauszman, A., Breitling, S., and 7 others 2018. “Experimental Right Ventricular Hypertension Induces Regional β1-integrin-mediated Transduction of Hypertrophic and Profibrotic Right and Left Ventricular Signaling,” Journal of the American Heart Association (Oxford, England) 7(7). pii: e007928; Baksaas-Aasen, K., Gall, L., Eaglestone, S., Rourke, C., Juffermans, N. P., and 10 others 2017. “iTACTIC-Implementing Treatment Algorithms for the Correction of Trauma-induced Coagulopathy: Study Protocol for a Multicentre, Randomised Controlled Trial,” Trials 18(1): 486; Barrett, C. D., Moore, H. B., Banerjee, A., Silliman, C. C., Moore, E. E., and Yaffe, M. B. 2017. “Human Neutrophil Elastase Mediates Fibrinolysis Shutdown through Competitive Degradation of Plasminogen and Generation of Angiostatin,” Journal of Trauma and Acute Care Surgery 83(6): 1053-1061; Bro-Jeppesen, J., Johansson, P. I., Kjaergaard, J., Wanscher. M., Ostrowski, S. R., Bjerre, M., and Hassager, C. 2017. “Level of Systemic Inflammation and Endothelial Injury is Associated with Cardiovascular Dysfunction and Vasopressor Support in Post-cardiac Arrest Patients,” Resuscitation 121:179-186; Moore, H. B., Moore, E. E., Huebner, B. R., Dzieciatkowska, M., Stettler, G. R., and 8 others 2017. “Fibrinolysis Shutdown is Associated with a Fivefold Increase in Mortality in Trauma Patients Lacking Hypersensitivity to Tissue Plasminogen Activator,” Journal of Trauma and Acute Care Surgery 2017 83(6): 1014-1022; Ono, Y., Hayakawa, M., Maekawa, K., Kodate, A., Sadamoto, Y., and 8 others 2017. “Fibrin/Fibrinogen Degradation Products (FDP) at Hospital Admission Predict Neurological Outcomes in Out-of-hospital Cardiac Arrest Patients,” Resuscitation 111:62-67; Trummer, G., Supady, A., Beyersdorf, F., Scherer, C., Wengenmayer, T., Umhau, M.5., and Benk, C. 2017. “Controlled Automated Reperfusion of the Whole Body after 120 Minutes of Cardiopulmonary Resuscitation: First Clinical Report,” Scandinavian Journal of Trauma, Resuscitation, and Emergency Medicine 25(1): 66; Wada. T., Gando, S., Mizugaki, A., Kodate, A., Sadamoto, Y., and 8 others 2017. “Differences in Coagulofibrinolytic Changes between Post-cardiac Arrest Syndrome of Cardiac Causes and Hypoxic Insults: A Pilot Study,” Acute Medicine and Surgery 4(3): 371-372; Wada. T. 2017. “Coagulofibrinolytic Changes in Patients with Post-cardiac Arrest Syndrome,” Online, Frontiers in Medicine (Lausanne, Switzerland) 4:156; Brinck, T., Handolin, L., and Lefering. R. 2016. “The Effect of Evolving Fluid Resuscitation on the Outcome of Severely Injured Patients: An 8-year Experience at a Tertiary Trauma Center,” Scandinavian Journal of Surgery 105(2): 109-116; Gerlach, H. 2016. “Agents to Reduce Cytokine Storm,” F1000Res [Faculty of 1000 Research Online] 5:2909; Hutin, A., Lamhaut, L., Lidouren, F., Kohlhauer, M., Mongardon, N., and 4 others 2016. “Early Coronary Reperfusion Facilitates Return of Spontaneous Circulation and Improves Cardiovascular Outcomes after Ischemic Cardiac Arrest and Extracorporeal Resuscitation in Pigs,” Journal of the American Heart Association (Oxford, England) 5(12). pii: e004588; Kiris. I., Kapan, S., Narin, C., Ozaydιn, M., Cure, M. C., Suteu R., and Okutan, H. 2016. “Relationship between Site of Myocardial Infarction, Left Ventricular Function, and Cytokine Levels in Patients Undergoing Coronary Artery Surgery,” Cardiovascular Journal of Africa 27(5): 299-306; Reynolds, P. S., Michael, M. J., Cochran, E. D., Wegelin, J. A., and Spiess, B. D. 2015. “Prehospital Use of Plasma in Traumatic Hemorrhage (The PUPTH Trial): Study Protocol for a Randomised Controlled Trial,” Trials 16:321; D'Elia, R. V., Harrison, K., Oyston, P. C., Lukaszewski, R. A., and Clark, G. C. 2013. “Targeting the “Cytokine Storm” for Therapeutic Benefit,” Clinical and Vaccine Immunology 20(3): 319-327; Radwan, Z. A., Bai, Y., Matijevic, N., del Junco, D. J., Mccarthy, J. J., and 3 others 2013. “An Emergency Department Thawed Plasma Protocol for Severely Injured Patients,” Journal of the American Medical Association Surgery 148(2): 170-175; Wada. T., Gando, S., Mizugaki, A., Yanagida, Y., Jesmin, S., Yokota, H., and leko, M. 2013. “Coagulofibrinolytic Changes in Patients with Disseminated Intravascular Coagulation Associated with Post-cardiac Arrest Syndrome-Fibrinolytic Shutdown and Insufficient Activation of Fibrinolysis Lead to Organ Dysfunction,” Thrombosis Research 132(1): e64-e69; Yiu, H. H., Graham, A. L., and Stengel, R. F. 2012. “Dynamics of a Cytokine Storm,” Online, PLoS [Public Library of Science] One 7(10): e45027; Alpert, M. A. 2011. “Sudden Cardiac Arrest and Sudden Cardiac Death on Dialysis: Epidemiology, Evaluation, Treatment, and Prevention,” Hemodialysis International 15 Supplement 1: S22-S29; Binks, A. and Nolan, J. P. 2010. “Post-cardiac Arrest Syndrome,” Minerva Anestesiologica 76(5): 362-368; Sun, M., Opavsky, M. A., Stewart, D. J., Rabinovitch, M., Dawood, F., Wen, W. H., and Liu, P. P. 2003. “Temporal Response and Localization of Integrins Beta1 and Beta3 in the Heart after Myocardial Infarction: Regulation by Cytokines,” Circulation 107(7): 1046-1052).

Whether due to an excessive increase in absolute stroke volume of either or both hearts, any such deviation in performance due to maladaptation or disease would originate past discharge from the hospital only to be counteracted upon detection by the implanted automatic response system. These safeguards and the usability of hearts that would otherwise be rejected for transplantation compensate for the extra space required to accommodate two hearts, and make a double heart transplant considerably preferable to a conventional heterotopic transplant, for example.

Then the implanted disorder response system would detect and automatically adjust the output pressure to fall within the normal range by electromechanical means addressed in U.S. Pat. No. 11,759,186, as well as herein, and/or pharmaceutical. Prior to any surgical procedure, an effort is made to predict and account for potential complications. Here this includes placement of the different type valves and shunts that might become necessary to adjust the pressure of inflow and ejection of the two hearts, determination of the drugs likely to be needed, and placement of the small flat reservoirs in the pectoral region from which the controller can command the release of the drugs.

Whereas in conventional orthotopic placement of an oversized heart (Trummer, G., Berchtold-Herz, M., Hillmann, R., Martin, J., and Beyersdorf, F. 2001. “Transplantation of an Oversized Heart,” Thoracic and Cardiovascular Surgery 49(6): 380-381) and in heterotopic, or ‘piggyback’ transplantation, a right and possibly a left pericardectomy is needed, to place a second intact heart within an unencroached space as preferred in the chest will likely require a right pericardectomy and a partial pneumonectomy (partial pneumectomy, partial pulmonectomy) of the lower right lung well inferior or caudal to the pulmonary arteries, pulmonary veins, and the diaphragm. Necessitating a clamshell thoracotomy or a bilateral thoracosternotomy (stemothoracotomy), the resection is accomplished at the same time as the transplant.

To allow free access to the vessels at either side of the heart for attachment of the servovalves, both orthotopic and double heart transplants require a clamshell thoracotomy or bilateral thoracosternotomy (see, for example, Pfannschmidt, J. 2015. “Thoracotomy and Stemotomy,” in Dienemann H., Hoffmann H., and Detterbeck F. (eds.), Chest Surgery, Springer Surgery Atlas Series, Berlin, Germany: Springer, pages 9-14) to allow. As addressed below in section 2b(1)(b)(b5), entitled Heterotopic Double Heart Metered Compound Vascular Bypass, or Sudden Switched-organ Transplantation [and so on], access thus is also needed in a double heart transplant when the second heart is to be positioned alongside to the left of the native heart, where a partial pneumonectomy is needed to afford adequate space.

Since with the apportionment of the workload between the two hearts is automatically adjusted by the implanted automatic response system, which can also directly target medication to either heart, a second heart positioned in the chest can be smaller, minimizing any postoperative outward or lateral protrusion or bulging Protection of the heart following placement of a second heart in the chest demands reinstatement of rib continuity. This is dealt with below in section 2b(1)(b)(b5), entitled Heterotopic Double Heart Metered Compound Vascular Bypass, or Sudden Switched-organ Transplantation [and so on].

Relief of the severe pain that follows a thoracotomy allows the patient to cough and thus expel secretions, with the result that infection and atelectasis, for example, which might otherwise arise as significant sequelae, are avoided (see, for example, Stamenkovic, D. M., Mladenovic, K., Rancic, N., Cvijanovic. V., Maric, N., and 4 others 2020. “Effect of Transcranial Direct Current Stimulation Combined with Patient-controlled Intravenous Morphine Analgesia on Analgesic Use and Post-thoracotomy Pain. A Prospective, Randomized, Double-blind, Sham-controlled, Proof-of-concept Clinical Trial,” Online, Frontiers in Pharmacology 11:125; Xing, X., Bai, Y., Sun, K., and Yan, M. 2020. “Single Nucleotide Polymorphisms Associated with Postoperative Inadequate Analgesia after Single-port VATS [video-assisted thoracoscopic surgery] in Chinese Population,” Online, BioMed Central Anesthesiology (London, England) 20(1):38; Wu, C. F., Hsieh, M. J., Liu, H. P., Gonzalez-Rivas, D., Liu, Y. H., and 3 others 2016. “Management of Post-operative Pain by Placement of an Intraoperative Intercostal Catheter after Single Port Video-assisted Thoracoscopic Surgery: A Propensity-score Matched Study,” Journal of Thoracic Disease 8(6): 1087-1093; Gerner P. 2008. “Postthoracotomy Pain Management Problems,” Anesthesiology Clinics 26(2): 355-367, vii.).

Management of the severe chest and often shoulder pain that ensues after a thoracotomy, or post-thoracotomy pain syndrome, demands minimizing nerve damage, avoiding incision into a bronchus, displacement of a scapula, and irritation from the thoracostomy tube or to the pericardium or mediastinal and diaphragmatic pleura. Chest pain is best ameliorated by thoracic epidural analgesia and/or paravertebral nerve block, serratus anterior plane block, pectoral nerve II block, erector spinae plane block and intercostal nerve block (see, for example, Fujii. S., Bairagi, R., Roche, M., and Zhou, J. R. 2019. “Transversus Thoracis Muscle Plane Block,” Online, BioMed Research International 2019:1716365; Gaber, S., Saleh, E., Elshaikh, S., Reyad, R., Elramly, M., Mourad, I., and Fattah, M. A. 2019. “Role of Perioperative Pregabalin in the Management of Acute and Chronic Post-thoracotomy Pain,” Open Access Macedonian Journal of Medical Sciences (Skopje. Macedonia) 7(12): 1974-1978; Kaushal, B., Chauhan, S., Saini, K., Bhoi. D., Bisoi, A. K., Sangdup, T., and Khan, M. A. 2019. “Comparison of the Efficacy of Ultrasound-guided Serratus Anterior Plane Block, Pectoral Nerves II Block, and Intercostal Nerve Block for the Management of Postoperative Thoracotomy Pain after Pediatric Cardiac Surgery,” Journal of Cardiothoracic and Vascular Anesthesia 33(2): 418-425; Ueshima, H., Otake, H., Hara. E., and Blanco, R. 2019. “How to Use Pectoral Nerve Blocks Effectively—An Evidence-based Update,” Asian Journal of Anesthesiology (Taipei, Taiwan) 2019:1-9; Vig. S., Bhan, S., Ahuja, D., Gupta, N., Kumar, V., Kumar, S., and Bharati, S. J. 2019. “Serratus Anterior Plane Block for Post-thoracotomy Analgesia: A Novel Technique for the Surgeon and Anaesthetist,” Indian Journal of Surgical Oncology 10(3): 535-539; Piraccini, E., Biondi, G., Byrne, H., Calli. M., Bellantonio, D., Musetti, G., and Maitan, S. 2018. “Ultrasound Guided Transversus Thoracic Plane block, Parasternal Block and Fascial Planes Hydrodissection for Internal Mammary Post Thoracotomy Pain Syndrome,” European Journal of Pain (London, England) 22(9): 1673-1677; Singh, P. M., Borle, A., Kaur, M., Trikha, A., and Sinha, A. 2018. “Opioid-sparing Effects of the Thoracic Interfascial Plane Blocks: A Meta-analysis of Randomized Controlled Trials,” Saudi Journal of Anaesthesia 12(1): 103-111; Chakraborty, A., Khemka, R., and Datta, T. 2016. “Ultrasound-guided Truncal Blocks: A New Frontier in Regional Anaesthesia,” Indian Journal of Anaesthesia 60(10): 703-711; Madabushi, R., Tewari, S., Gautam, S. K., Agarwal, A., and Agarwal, A. 2015. “Serratus Anterior Plane Block: A New Analgesic Technique for Post-thoracotomy Pain,” Pain Physician 18(3): E421-E424; Fortier, S., Hanna, H. A., Bernard, A., and Girard, C. 2012. Comparison between Systemic Analgesia, Continuous Wound Catheter Analgesia and Continuous Thoracic Paravertebral Block: A Randomised Controlled Trial of Postthoracotomy Pain Management,” European Journal of Anesthesiology 29(11): 524-530; Conlon, N. P., Shaw, A. D., and Grichnik, K. P. 2008. “Postthoracotomy Paravertebral Analgesia: Will it Replace Epidural Analgesia?,” Anesthesiology Clinics 26(2): 369-380, viii.; Gemner P. 2008. Op cit.).

That a heart transplant using the switch technique, or any other extracardiac procedure, such as a coronary artery bypass graft or a partial pneumonectomy can be accomplished without general anesthesia or cardiopulmonary bypass, and without collapse of the lungs warrants explanation. Entry into the chest results in the admission of air into the pleural cavity causing lung collapse, or pneumothorax. Often a lung operated upon is allowed to collapse, the patient continuing to breathe with the isolated contralateral lung, with mechanical ventilation assistance as necessary.

While single-lung breathing eliminates the need for direct mechanical oxygenation of the blood-which does not, as does direct blood oxygenation, damage blood cells but nevertheless may lead to further complications, here, double heart metered compound vascular bypass, or sudden switched-organ transplantation addressed in section 2b(1)(b)(b5), entitled Heterotopic Double Heart Metered Compound Vascular Bypass, or Sudden Switched-organ Transplantation [and so on], and heart-lung transplantation addressed in section 2b(1)(b)(b10), entitled Metered Compound Vascular Bypass, or Switched-organ, Heart-Lung Transplantation, are best performed without or minimal artificial support.

By collapsing both lungs, the usual full chest clamshell necessitates the use of a surgical chest dome as described below in section 2b(2)(k) entitled Surgical Chest Dome, or artificial ventilation (see, for example, Pompeo, E. 2012. “Awake Thoracic Surgery—Is It Worth the Trouble?,” Seminars in Thoracic and Cardiovascular Surgery 24(2): 106-114; Erkmen, C. P., Ducko. C. T., and Jaklitsch, M. T. 2009. “Thoracic Incisions,” Part 1, Chapter 2 in Sugarbaker, D. J., Bueno, R., Krasna, M. J. Mentzer, S. J., and Zellos, L., Adult Chest Surgery, New York, New York: McGraw Hill; Brown, L. M., Rannels, S. R., and Rannels, D. E. 2001. “Implications of Post-pneumonectomy Compensatory Lung Growth in Pulmonary Physiology and Disease,” Respiratory Research 2(6): 340-347; Bains, M. S., Ginsberg, R. J., Jones, W. G. 2nd, McCormack, P. M., Rusch, V. W., Burt, M. E., and Martini, N. 1994. “The Clamshell Incision: An Improved Approach to Bilateral Pulmonary and Mediastinal Tumor,” Annals of Thoracic Surgery 58(1): 30-33).

The pleural cavities separate (see, for example, Burstiner, L. and Al Khalili, Y. 2019. “Anatomy, Thorax, Pleurae,” Online, Treasure Island, Florida: StatPearls Publishing Co., at https://www.ncbi.nlm.nih.gov/books/NBK541079/; Charalampidis. C., Youroukou, A., Lazaridis, G., Baka, S., Mpoukovinas, I., and 14 others 2015. “Pleura Space Anatomy,” Journal of Thoracic Disease 7 (Supplement 1): S27-S32; Lee, K. F. and Olak, J. 1994. “Anatomy and Physiology of the Pleural Space,” Chest Surgery Clinics of North America 4(3): 391-403), surgical entry into the chest allows air to enter collapsing the lung on that side but leaving the contralateral lung unaffected and usable.

However, a clamshell thoracotomy spans the chest, causing both lungs to collapse, thus disallowing the use of one-lung or selective lobar surgery with mask or intubated ventilation to oxygenate a patient under conscious sedation unable to breathe. Spontaneous breathing under regional anesthesia is preferable to the use of artificial, or mechanical, ventilation. Open-heart, or intracardiac, procedures include conventional but not switch method executed heart or heart and lung transplantation and the repair of less severe congenital malformities where the repair will result in normal circulation. Coronary artery plaque is least traumatically removed transluminally, most often by balloon angioplasty. Improved means for removing rather than crushing plaque are described in U.S. Pat. No. 11,389,171 entitled Integrated System for the Infixion and Retrieval of Implants.

With a surgical chest dome, described in this section and below in section 2b(2)(k), entitled Surgical Chest Dome, and section 5, entitled Description of the Drawing Figures, essentially a cross between a laboratory or industrial glovebox and an irregularly shaped vacuum bell jar used to enclose harvested lungs during ex vivo lung perfusion, for example, but provided with large diameter airtight gloved openings for surgical use, intracardiac procedures require only cardio-, not pulmonary bypass. By maintaining equal intra- and extrapulmonary gas pressures during thoracic surgery, the chest dome prevents a unilateral or a bilateral pneumothorax, thus allowing the use of natural breathing, or spontaneous ventilation, under regional if not local anesthesia (see, for example, Chakravarthy, M. 2018. “Regional Analgesia in Cardiothoracic Surgery: A Changing Paradigm toward Opioid-free Anesthesia?,” Annals of Cardiac Anaesthesia (New Delhi, India) 21(3): 225-227).

Currently the norm, when a graft organ becomes available after death, the concept of ex vivo lung perfusion can be applied to any organ to allow pre-transplantation therapy. However, even if this includes immune tolerance induction through the infusion of blood products from the prospective recipient, for example, that the organ has been severed from a normal physiological milieu severely reduces the value of such treatment (see also section 2a(3) on remote preservation). The optimal form of pre-transplantation therapy is that delineated herein, whereby the end-stage patient has been placed on life support, if possible, with normal breathing, or spontaneous ventilation, and circulation administered by an automatic implanted system as described herein, in advance of the terminal event so that essentially normal physiology has continued without avoidable disruption.

When the prospective donor is found shortly after death, the best option is to preserve the anatomy intact and reinitiate circulation and respiration administered by an implanted automatic system as described. That only a finite amount of time will allow this is a protective factor. The worst approach is to sever organs from the body and treat these apart from the context in which these developed and functioned. The surgical chest dome is intended for use during any procedure, endoscopic, video-assisted thorascopic, hemiclamshell, or clamshell, to equalize the pressure outside the chest where a lung collapse would best be avoided. Broadly, any procedure that might cause a pneumothorax, such as an endoscopic lobectomy, can avoid this eventuality with the aid of a chest dome.

Absent a pressure equalizing chest dome, a clamshell thoracotomy, for example causes both lungs to collapse, precluding spontaneous, and thus requiring mechanical ventilation. A chest dome offers the advantages of allowing any extracardiac procedure to be performed without the need for general anesthesia, cardioplegia, or cross-clamping, each of which is associated with serious sequelae. Unlike a laboratory or industrial glove box, the chest dome is provided with more than two large diameter airtight gloved entry portals where each allows unrestrained freedom of movement to allow operators clear and ready access to any enclosed point from the front, back, or sides.

To allow operating team members to pass tools and tubing in and out of the chest dome while minimizing if not preventing air from entering, the chest dome has high density bristle antistatic brush ‘air curtains’ or windows so that many bristles, each stiff enough not to bend due to the pressure difference, remain in contact with, running along the surface of the item passed, blocking air from entry. Briefly, molded as a clear transparent tough plastic shell made of bisphenol A and plasticizer-free abrasion, chemical, solvent, and microbial-resistant non-yellowing thermally cured silicone, or polysiloxane-coated polycarbonate sheet, for example, the surgical chest dome encloses the space above and to the sides of the chest to allow equalization of extra- and intrapulmonary pressures.

The bottom of the shell is positioned below the diaphragm, that is, sufficiently inferior along the body as not to restrict the diaphragm in breathing. The chest dome rests upon, fully encloses, and seals off the chest from the surrounding air, and bends downward at the sides to rest upon and place much of its weight on the table. Unadjustable, the chest dome must be made available in different sizes. To reduce the height and weight of the chest dome, the padded arms of the supine patient, are placed with the hands behind the head rather than upwards to expose the axillae (see FIG. 1 in Hayanga, J. W. and D'Cunha, J. 2014. “The Surgical Technique of Bilateral Sequential Lung Transplantation,” Journal of Thoracic Disease 6(8): 1063-1069).

Precautionary measures specified by Hayanga, J. W. et al. apply. An airtight thick viscoelastic polyurethane foam base surrounding a cast iron core is bonded to the bottom of the dome. The light vacuum should seldom require a coating about the base of vacuum grease. Construction thus is intended to assure self-seating and self-sealing thus eliminating the need for straps that lock beneath the table, impeding both positioning and removal. A more detailed description of a dome is provided in section 2d, entitled Body Surface Side-entry Ports.

The large gloved openings afford sufficient maneuverability to allow operators to move their hands up or down, forward, or sideways. Surgical tools, tubing, retractors, and so on are prepositioned inside the dome which also incorporates one or more brush-surround ‘air curtains’ to conserve the mild vacuum while other items are passed through as necessary. In a heart-lung transplant using the sudden or metered switch method, due to the size of the graft heart-lung bloc, a midprocedural pneumothorax can be avoided by placing recipient and donor in close adjacency under a common to both, or double-wide, surgical chest dome which extends over to include the space between the two operating tables.

For ease in transferring the harvested graft heart-lung bloc to, and removing the native bloc from the recipient, the double chest dome provides large diameter airtight front, rear, and side, glove openings and brush-surround air windows in the section connecting the two as well as at either end. In single heart, lung, or thyroid transplant which might result in a problematic pneumothorax, the relatively brief interval needed to move the graft from under the donor chest dome and into that of the neighboring recipient and to remove the native organ allows requires separate chest domes or one double-wide to cover two subjects with large glove holes to allow removal and entry of the hearts. For this reason, double and triple models to extend over more than a single subject in order to prevent pneumothorax are made available for pneumothorax-intolerant patients.

In a double, or bilateral simultaneous, lung transplant with a live or deceased single lung or lobe donor to either side, a triple configuration to span over the recipient at the center can be provided. Relatively lengthy collapse routinely occurs during single lung unassisted breathing, or spontaneous ventilation, as surgical, that is, intentional, or iatrogenic, pneumothorax, and the time to transfer a harvested lung from the donor to the recipient chest dome alongside the donor is a matter of moments. Therefore, except in large centers where the volume of such operations may eventually increase to justify double and triple patient domes, single patient domes with self-adjusting suction pump to maintain a light vacuum to keep intra- and extrapulmonary pressures equal inside the dome should prove satisfactory.

Neither does a momentary collapse justify complicating the use of individual chest domes through the addition of a dome-to-dome connecting ‘skybridge,’ or side-tunnel with glove openings, for example. It is also possible to counteract a pneumothorax by increasing and sustain the volume of air inspired (see, for example, Kenny, B. J. and Ponichtera, L. 2019. “Physiology, Boyle's Law,” Treasure Island, Florida: StatPearls Publishing, Online, at https://www.ncbi.nlm.nih.gov/books/NBK538183/; Sharma, S., Hashmi, M. F., and Rawat, D. 2019. “Partial Pressure of Oxygen (PO2),” Online, Treasure Island, Florida: StatPearls Publishing Co., at https://www.ncbi.nlm.nih.gov/books/NBK493219).

Ventilation is preferably spontaneous with standby mechanical assistance if needed (see, for example, Tan, J., Song, Z., Bian, Q., Li, P., and Gu, L. 2018. “Effects of Volume-controlled Ventilation vs. Pressure-controlled Ventilation on Respiratory Function and Inflammatory Factors in Patients Undergoing Video-assisted Thoracoscopic Radical Resection of Pulmonary Carcinoma,” Journal of Thoracic Disease 10(3): 1483-1489; Ghabach, M. B., El Haji, E. M., El Dib, R. D., Rkaiby, J. M., Matta, M. S., and Helou, M, R. 2017. “Ventilation of Nonparalyzed Patients under Anesthesia with Laryngeal Mask Airway, Comparison of Three Modes of Ventilation: Volume Controlled Ventilation, Pressure Controlled Ventilation, and Pressure Controlled Ventilation-volume Guarantee,” Anesthesia, Essays and Researches (Mumbai [Bombay] India) 11(1): 197-200; Zhu, Y. Q., Fang, F., Ling, X. M., Huang, J., and Cang, J. 2017. “Pressure-controlled versus Volume-controlled Ventilation during One-lung Ventilation for Video-assisted Thoracoscopic Lobectomy,” Journal of Thoracic Disease 9(5): 1303-1309; Kim, K. N., Kim, D. W., Jeong, M. A., Sin, Y. H., and Lee, S. K. 2016. “Comparison of Pressure-controlled Ventilation with Volume-controlled Ventilation during One-lung Ventilation: A Systematic Review and Meta-analysis,” Online, BioMed Central Anesthesiology 16(1): 72; David, P., Pompeo, E., Fabbi, E., and Dauri, M. 2015. “Surgical Pneumothorax under Spontaneous Ventilation-effect on Oxygenation and Ventilation,” Annals of Translational Medicine 3(8): 106; Lin, F., Pan, L., Qian, W., Ge, W., Dai, H., and Liang, Y. 2015. “Comparison of Three Ventilatory Modes during One-lung Ventilation in Elderly Patients,” Hu, X., Shen, H., Li, X., and Chen, J. 2014. “Effects of Volume-controlled Ventilation and Pressure-controlled Volume-Guaranteed Mode during One-lung Ventilation on Circulation, Pulmonary Function and Lung Injury,” (in Chinese with abstract in English at Pubmed), Zhonghua Yi Xue Za Zhi [Chinese Medical Journal] 94(13): 1006-1009; Lin, F., Pan, L., Huang, B., Ruan, L., Liang, R., Qian, W., and Ge, W. 2014. “Pressure-controlled versus Volume-controlled Ventilation during One-lung Ventilation in Elderly Patients with Poor Pulmonary Function,” Armals of Thoracic Medicine 9(4): 203-208; Song, S. Y., Jung, J. Y., Cho, M. S., Kim, J. H., Ryu, T. H., and Kim, B. I. 2014. “Volume-controlled versus Pressure-controlled Ventilation-volume Guaranteed Mode during One-lung Ventilation,” Korean Journal of Anesthesiology 67(4): 258-263; Pompeo, E. 2012, Op cit., this section; Campbell, R. S. and Davis, B, R. 2002. “Pressure-controlled versus Volume-controlled Ventilation: Does It Matter?,” Respiratory Care 47(4): 416-426; Cafiero, F., Sturlese, P., Vergani, F., and Ciapponi, A. 1955. “Surgical Pneumothorax of Short Duration as a Part of Conservative Thoracoplasty,” (in Italian), Giornale Italiano della Tubercolosi e Delle Malattie del Torace [Italian Journal of Tuberculosis and Chest Diseases] 9(1): 31-41). For this reason, double and triple models to prevent pneumothorax may be sought only for pneumothorax-intolerant patients. References pertaining to single lung, or one-lung ventilation proper and its complications appear below in this section.

Using the methods to be described herein for:
1. Extracardiac surgery, cardiopulmonary bypass is not required, and for
2. Intracardiac surgery, only the cardiopulmonary bypass roller or centrifugal pump with tubing, cannulae, and the cardioplegia circuit are required, not the oxygenator, and
3. The need for general anesthesia with either extracardiac or intracardiac in terms of analgesia if not anxiolysis, or conscious sedation, is eliminated for both, and
4. Unless an intracardiac repair will restore normal circulation, a transplant is preferable, and performed as described herein, will be safer, less traumatic, and with increased availability of transplantable hearts, the number of intracardiac procedures and therefore the need for cardiopulmonary bypass should diminish.

Furthermore, a severe Type A aortic dissection, aneurysm, or especially difficult valve repair in an elderly patient with significant comorbidity, for example, is far more safely managed by not attempting to repair the defect but rather by replacing the heart along with an intact aorta and/or valves (see, for example, Fukui, T. 2018. “Management of Acute Aortic Dissection and Thoracic Aortic Rupture,” Journal of Intensive Care 6:15; Stamou, S. C., Rausch, L. A., Kouchoukos, N. T., Lobdell, K. W., Khabbaz, K., Murphy, E., and Hagberg, R, C. 2016. “Comparison between Antegrade and Retrograde Cerebral Perfusion or Profound Hypothermia as Brain Protection Strategies during Repair of Type A Aortic Dissection,” Annals of Cardiothoracic Surgery 5(4): 328-335).

The same pertains to a congenitally malformed heart where any attempt at repair would still not achieve normal circulation. Given the increase in available hearts made possible by an implanted automatic response system, many marginal hearts previously rejected can be transplanted, and double heart transplantation further increases the number. Any transluminal or transcatheteric means for the repair of an intracardiac defect without the need to open the chest, such as vulnerable plaque in a coronary artery near to its origin or a heart valve where the result should prove curative and durable likewise avoids the need for general anesthesia, cardioplegia, and so on, and does so with the least trauma-such means are always to be preferred.

Invasive extracardiac and pulmonary surgery can be performed under regional anesthesia or analgesia without either lung collapsed and with the patient breathing normally as preferred (see, for example, Elkhayat, H. and Gonzalez-Rivas, D. 2019. “Non-intubated Uniportal Video-assisted Thoracoscopic Surgery,” Journal of Thoracic Disease 11 (Supplement 3): S220-S222; Solli, P., Brandolini, J., and Bertolaccini, L. 2019. “Tubeless Thoracic Surgery: Ready for Prime Time?,” Journal of Thoracic Disease 11(3): 652-656; Ahn, S., Moon, Y., AlGhamdi, Z. M., and Sung, S. W. 2018. “Nonintubated Uniportal Video-assisted Thoracoscopic Surgery: A Single-center Experience,” Korean Journal of Thoracic and Cardiovascular Surgery 51(5): 344-349; AlGhamdi, Z. M., Lynhiavu, L., Moon, Y. K., Moon, M. H., Ahn, S., Kim, Y., and Sung, S. W. 2018. “Comparison of Non-intubated versus Intubated Video-assisted Thoracoscopic Lobectomy for Lung Cancer,” Journal of Thoracic Disease 10(7): 4236-4243) or ventilator-assisted with a chest surgery glovebox, or surgical chest dome, described in this section, 2a, and below in sections 2b(2)(g), entitled Ischemia and Hypoperfusion-free Carotid Endarterectomy Bypass and Prosthesis, and 6, entitled Description of the Preferred Embodiments of the Invention.

In rendering heart transplantation an extracardiac procedure and eliminating the need for general anesthesia, cardioplegia, and intubated oxygen delivery, single or double switch heart transplantation eliminates the risk of serious complications, especially during gestation and delivery. In an infant, slightly less than one hour under general anesthesia and awake-regional anesthesia result in no meaningful distinction in neurocognitive development by five years old. Once implemented, switch transplantation will allow a curtailment in intracardiac procedures to include only those known to repair congenital heart defects so that circulation will be normal.

Because a congenitally abnormal heart initiates degradation in all the organs and tissues of the body, a malformed heart should be replaced in utero. And because extracardiac, switch transplantation allows the removal and replacement of the defective heart intact, more safely and less traumatically, without intricate intracardiac microsurgery, the operation will be far more likely to succeed-without cardiopulmonary bypass, cardioplegia, or general anesthesia. However, procedures of the complexity and intricacy contemplated herein will usually take more than one hour, often considerably more, anesthetization may be required more than once, and general anesthesia carries numerous risks unrelated to neurodevelopment. For present purposes, general anesthesia in infancy is best avoided.

The literature overall veers toward a preference for the avoidance of general anesthesia (McCann, M. E., de Graaff, J. C., Dorris, L., Disma, N., Withington, D., and 23 others with 76 collaborators 2019. “Neurodevelopmental Outcome at 5 Years of Age after General Anaesthesia or Awake-regional Anaesthesia in Infancy (GAS): An International, Multicentre, Randomised, Controlled Equivalence Trial,” Lancet 393(10172): 664-677; Xu, J., Xu, M., Wang, Y., Mathena, R. P., Wen, J., and 3 others 2019. “Anesthetics Disrupt Growth Cone Guidance Cue Sensing through Actions on the GABAA α2 Receptor Mediated by the Immature Chloride Gradient,” Neurotoxicology and Teratology 74:106812; Davidson, A. J., Disma, N., de Graaff, J. C., Withington, D. E., Dorris, L., and 26 others with 102 collaborators 2016. “Neurodevelopmental Outcome at 2 Years of Age after General Anaesthesia and Awake-regional Anaesthesia in Infancy (GAS): An International Multicentre, Randomised Controlled Trial,” Lancet 387(10015): 239-250; Frawley, G., Bell, G., Disma, N., Withington, D. E., de Graaff, J. C., and 7 others with 100 collaborators 2015. “Predictors of Failure of Awake Regional Anesthesia for Neonatal Hernia Repair: Data from the General Anesthesia Compared to Spinal Anesthesia Study-Comparing Apnea and Neurodevelopmental Outcomes,” Anesthesiology 123(1): 55-65).

Complications in otherwise healthy, sick, immature, and pregnant patients caused by general anesthesia are infrequent in incidence but numerous in kind (Hepner, A., Negrini, D., Hase, E. A., Exman, P., Testa, L., and 6 others 2019. “Cancer during Pregnancy: The Oncologist Overview,” World Journal of Oncology 10(1): 28-34; Smith, G. and Goldman, J. 2019. “General Anesthesia for Surgeons,” Online, Treasure Island, Florida: StatPearls Publishing Co., at https://www.ncbi.nlm.nih.gov/books/NBK493199/; Bhatia, P. and Chhabra, S. 2018. “Physiological and Anatomical Changes of Pregnancy: Implications for Anaesthesia,” Indian Journal of Anaesthesia 62(9): 651-657; Botha, M. H., Rajaram, S., and Karunaratne, K. 2018. “Cancer in Pregnancy,” International Journal of Gynaecology and Obstetrics 143 Supplement 2:137-142; Kodali, B. S. and Bharadwaj, S. 2018. “Foetal Surgery: Anaesthetic Implications and Strategic Management,” Indian Journal of Anaesthesia 62(9): 717-723: Olutoye, O. A., Baker, B. W., Belfort, M. A., and Olutoye, O. O. 2018. “Food and Drug Administration Warning on Anesthesia and Brain Development: Implications for Obstetric and Fetal Surgery,” American Journal of Obstetrics and Gynecology 218(1): 98-102; Pucher, B., Szydlowski, J., Jonczyk-Potoczna, K., and Sroczynski, J. 2018. “The EXIT (Ex- utero Intrapartum Treatment) Procedure—from the Paediatric ENT Perspective,” Acta Otorhinolaryngologica Italica 38(5): 480-484; Ravindra, G, L., Madamangalam, A. S., and Seetharamaiah, S. 2018. “Anaesthesia for Non-obstetric Surgery in Obstetric Patients,” Indian Journal of Anaesthesia 62(9): 710-716; Armstrong, R., Xu, F., Arora, A., Rasic, N., and Syed, N. I. 2017. “General Anesthetics and Cytotoxicity: Possible Implications for Brain Health,” Drug and Chemical Toxicology 40(2): 241-249; Fodale. V., Tripodi. V. F., Penna, O., Fama, F., Squadrito, F., Mondello. E., and David, A. 2017. “An Update on Anesthetics and Impact on the Brain,” Expert Opinion on Drug Safety 16(9): 997-1008; Vutskits, L. and Davidson, A. 2017. “Update on Developmental Anesthesia Neurotoxicity,” Current Opinion in Anesthesiology 30(3): 337-342; Upadya, M. and Saneesh, P. J. 2016. “Anaesthesia for Non-obstetric Surgery during Pregnancy,” Indian Journal of Anaesthesia 60(4): 234-241; Harris, M. and Chung, F. 2013. “Complications of General Anesthesia,” Clinics in Plastic Surgery 40(4): 503-513; Rabinstein, A, A. and Keegan, M. T. 2013. “Neurologic Complications of Anesthesia: A Practical Approach,” Neurology. Clinical Practice 3(4): 295-304).

Inseparable from the potential complications associated with general anesthesia are those attributable to intubation (see, for example, Dadure, C., Sabourdin, N., Veyckemans, F., Babre, F., Bourdaud, N., and 14 others 2019. “Management of the Child's Airway under Anaesthesia: The French Guidelines,” Anaesthesia, Critical Care and Pain Medicine pii: S2352-5568(19) 30070-0; Langeron, O., Bourgain, J, L., Francon, D., Amour, J., Baillard, C., and 8 others 2018. “Difficult Intubation and Extubation in Adult Anaesthesia,” Anaesthesia, Critical Care and Pain Medicine 37(6): 639-651; Huitink, J. M., Lie, P. P., Heideman, I., Jansma, E. P., Greif, R., van Schagen, N., and Schauer, A. 2017. “A Prospective, Cohort Evaluation of Major and Minor Airway Management Complications during Routine Anaesthetic Care at an Academic Medical Centre,” Anaesthesia 72(1): 42-48; Steadman, J., Catalani. B., Sharp. C., and Cooper. L. 2017. “Life-threatening Perioperative Anesthetic Complications: Major Issues Surrounding Perioperative Morbidity and Mortality,” Trauma Surgery and Acute Care Open 2(1): e000113; Mitchell, J., Clement de Clety, S., Collard. E., De Kock. M., Detaille. T., and 4 others 2016. “Unplanned Intensive Care Unit Admission after General Anaesthesia in Children: A Single Centre Retrospective Analysis,” Anaesthesia, Crit Care and Pain Medicine 35(3): 203-208; Cook, T. M., Woodall, N., and Frerk, C. 2011. “Major Complications of Airway Management in the UK: Results of the Fourth National Audit Project of the Royal College of Anaesthetists and the Difficult Airway Society. Part 1: Anaesthesia,” British Journal of Anaesthesia 106(5): 617-631).

In compound vascular bypass heart transplantation, during which both recipient and donor hearts remain continuously perfused, the elimination of cardioplegia reduces the scope of possible serious complications. Used in conjunction with anesthesia and cross-clamping, the attribution to cardioplegia of adverse complications in isolation from these other factors is seldom distinct. An exception is when the method used to introduce the solution is the cause. On rare occasion, retrograde cardioplegia-when a calcium-free hyperkalemie solution containing magnesium and lidocaine, for example, is introduced into the coronary sinus-results in sinus rupture or perforation of the inner wall of the right atrium, considerably extending the duration of the procedure and increasing the odds for an adverse outcome.

The use of cardioplegia in the recipient is stated to “ . . . yield a lower prevalence of right heart failure, arrhythmias, and myocardial ischemia early after [conventional orthotopic] heart transplantation,” (Luciani, G. B., Fomi, A., Rigatelli, G., Chiominto, B., Cadaioli, P. Mazzucco, A., and Faggian, G. 2011. “Myocardial Protection in Heart Transplantation Using Blood Cardioplegia: 12-year Outcome of a Prospective Randomized Trial,” Journal of Heart and Lung Transplantation (New York, New York) 30(1): 29-36). Cardioplegia solution is also used in the graft organ and in the recipient in a conventional heterotopic heart transplant (Shumway, S. Bolman, R. M. 1997. “Cardiac Transplantation,” in Chapter 16, Greenfield, L. J., Mulholland, M., Oldham, K. T., Zelenock, G. B., and Lillemoe, K. D., (eds.) Surgery: Scientific Principles and Practice, Philadelphia, Pennsylvania: Lippincott Raven, pages 600, 602), and used as well as a continuously chilled static or flowing perfusate, or perfusant, preservative superior to the hypothermic immersion technique administered prior to harvesting and given at intervals or continuously during preservation (see, for example, Smulowitz, P. B., Serna, D, L., Beckham, G. E., and Milliken, J, C. 2000. “Ex Vivo Cardiac Allograft Preservation by Continuous Perfusion Techniques,” Online, American Society for Artificial Internal Organs Journal 46(4): 389-396) (see also below under section 2a(3), entitled Remotely Procured Graft Organ Preservation)

However, the elimination of cardioplegia from heart transplantation using any method avoids the potential for numerous complications and dependency upon the expertise of the indivdiaul practitioner (see, example, Carvajal, C., Goyal, A., and Tadi, P. 2022. “Cardioplegia,” Online, Treasure Island, Florida: StatPearls Publishing Co.; Ismail, A. and Semien, G. 2022. “Myocardial Protection,” Online, Treasure Island, Florida: StatPearls Publishing Co.; Ismail, A. Semien, G., and Miskolczi, S. Y. 2022. “Cardiopulmonary Bypass,” Online, Treasure Island, Florida: StatPearls Publishing Co.; Kamassai, J, D. and Lower, D, R. 2022. “Retrograde Cardioplegia,” Online, Treasure Island, Florida: StatPearls Publishing Co.; Karaaslan, K., Abud, B., and Karacelik, M., 2021. “Cardioplegia Application with a Hand-squeezed Cardioplegia Bag. Is It Safe?,” Heart Surgery Forum 24(4): E619-E623; Siddiqi, S., Blackstone, E. H., and Bakaeen, F, G. 2018. “Bretschneider and del Nido Solutions: Are They Safe for Coronary Artery Bypass Grafting? If So, How Should We Use Them?,” Journal of Cardiac Surgery 33(5): 229-234; Sobieraj. M., Kilanowska. M., Ładziński, P., Garbuzowa, I., Wojtalik, M., Moczko, J., and Mrówczyński, W. 2018. “Type of Cardioplegic Solution as a Factor Influencing the Clinical Outcome of Open-heart Congenital Procedures,” Kardiochirurgia i Torakochirurgia Polska [Polish Journal of Cardiothoracic Surgery] 15(2): 86-94; Potz. B, A. Scrimgeour, L. A., Feng, J., and Sellke, F. W.2017. “Diabetes and Cardioplegia,” Online, Journal of Nature and Science 3(6): e394; Lee, C. H., Ju, M. H., Kim, J. B., Chung, C. H., Jung, S. H., Choo, S. J., and Lee, J. W. 2014. “Myocardial Injury following Aortic Valve Replacement for Severe Aortic Stenosis: Risk Factor of Postoperative Myocardial Injury and Its Impact on Long-term Outcomes,” Korean Journal of Thoracic and Cardiovascular Surgery 47(3): 233-239; Maruyama, Y., Chambers, D. J., and Ochi, M. 2013. “Future Perspective of Cardioplegic Protection in Cardiac Surgery,” Journal of Nippon Medical School 80(5): 328-341; Fujii. T., Watanabe, Y., Shiono, N., Kawasaki, M., Yokomuro, H., and 8 others 2006. “Limitations of Retrograde Continuous Tepid Blood Cardioplegia for Myocardial Remodeling,” Annals of Thoracic and Cardiovascular Surgery 12(6): 397-403; He, G. W. 1997. “Rare Complication of Retrograde Cardioplegia: Inner Wall Perforation of the Right Atrium,” Annals of Thoracic Surgery 63(2): 539-541; Katayama, O., Amrani, M., Ledingham, S., Jayakumar, J., Smolenski, R. T., and 3 others 1997. “Effect of Cardioplegia Infusion Pressure on Coronary Artery Endothelium Cardiac Mechanical Function,” European Journal of Cardiothoracic Surgery 11(4): 751-762; Allen, B. S., Winkelmann, J. W., Hanafy, H., Hartz, R. S., Bolling, K. S., Ham, J., and Feinstein, S. 1995. “Retrograde Cardioplegia Does Not Adequately Perfuse the Right Ventricle,” Journal of Thoracic and Cardiovascular Surgery 109(6): 1116-1126; Winkelmann, J., Aronson, S., Young, C. J., Fernandez, A., and Lee, B. K. 1995. “Retrograde-delivered Cardioplegia is Not Distributed Equally to the Right Ventricular Free Wall and Septum,” Journal of Cardiothoracic and Vascular Anesthesia 9(2): 135-139; Mullen, J. C., Khan, N., Weisel, R. D., Christakis, G. T., Teoh, K. H., and 3 others 1987. “Atrial Activity during Cardioplegia and Postoperative Arrhythmias,” Journal of Thoracic and Cardiovascular Surgery 94(4): 558-565).

Compound vascular bypass, or switched-organ transplantation eliminates ischemia and the complication associated with it. The use of cardioplegia, for example, seeks to minimize ischemia-reperfusion injury; however, zero ischemia means zero ischemia-reperfusion injury. While beneficial immediately before implantation following a long graft ischemic time, elimination of the need for cardioplegia during heart transplantation avoids a source of potentially serious complications (Luciani, G. B., Forni, A., Rigatelli, G., Chiominto, B., Cadaioli, P. Mazzucco, A., and Faggian, G. 2011. Op cit., third paragraph above in this section.

In the context of open heart procecures and coronary artery surgery generally, cardioplegia can be the cause of complications. In a patient with aortic valve stenosis and mitral and tricuspid insufficiency, complications with selective antegrade cardioplegia can be quite serious (see, for example, Di Mauro, M., Calafiore, A M., Di Franco, A., Nicolini, F., Formica, F., and 14 others 2021. “Association between Cardioplegia and Postoperative Atrial Fibrillation in Coronary Surgery,” Online, International Journal of Cardiology 324:38-43; Balkoussis, N. G., Papakonstantinou, N. A., Verra, C., Kakouris, G., Chounti, M., and 3 others 2015. “Mechanisms of Oxidative Stress and Myocardial Protection during Open-hear Surgery,” Online, Annals of Cardiac Anesthesia 18(4): 555-564).

The wider benefits of eliminating ischemia where conventional techniques would otherwise call for cardiopulmonary bypass and cardioplegia include other procedures where these are used, to include extracardiac correction of the great vessels, as addressed below in sections 2b(2)(a), entitled Transectionless and ischemia-free Correction of Complete, or Dextro-transposition, of the Great Arteries, 2b(2)(d), entitled Anoxia-free Aortic Repair: Aortic Prostheses Thoracic, Thoracoabdominal, and Abdominal, and 2b(2)(g), entitled Ischemia and Hypoperfusion-free Carotid Endarterectomy Bypass and Prosthesis.

A dissection of the left coronary main stem can complicate cardioplegia resulting in a myocardial infarction and death (see, for example, Carvajal, C., Goyal, A. and Tadi, P. 2022, Op. cit., third paragraph above in this section; Sato, S., Azami, T., Kawamoto, T., Inoue, K., and Okada, K. 2022. “Safety and Applicability of Continuous Retrograde Cardioplegia in Minimally Invasive Aortic Valve Replacement: New Approaches,” Online, Annals of Thoracic and Cardiovascular Surgery 28(1): 36-40; Kim, H. R., Jung, S. H., Yang, J., Kim, M. S., Yun, T. J., Kim, J. J., and Lee, J. W. 2020. “The Effect of Supplemental Cardioplegia Infusion before Anastomosis in Patients Undergoing Heart Transplantation with Long Ischemic Times,” Online, Korean Journal of Thoracic and Cariovascular Surgery 53(6): 375-380; Ribeiro, R. V. P., Friedrich, J. O., Ouzounian, M., Yau. T., Lee, J., and Yanagawa, B. 2020. “Supplemental Cardioplegia during Donor Heart Imsplantation: A Systematic Review and Meta-analysis,” Online, Annals of Thoracic Surgery 110(2): 545-552; Tevaearai Stahel, H. T., Unger, D., Schmidli, J., Gahl. B., Englberger, L., and 4 others 2014. “Supplemental Cardioplegia Immediately before Graft Implantation May Improve Early Post-transplantation,” Outcome, Online, Frontients in Surgery 1:46; van Putte, B. P., Vink, A., De Bruin, P. C., and Defauw. J. J. 2007. “Selective Antegrade Cardioplegic Perfusion Complicated by Left Main Stem Dissection,” Journal of Cardiovascular Surgery (Turin, Italy) 48(2): 247-248)(see also Ahmed, A, A. and Mahboobi, S. K. 2022. “Warm Blood Cardioplegia,” Online, Treasure Island, Florida: StatPearls Publishing Co.; Fremes, S. E., Weisel, R. D., Mickle, D. A., Ivanov, J., Madonik, M. M., and 4 others 1985. “Myocardial Metabolism and Ventricular Function following Cold Potassium Cardioplegia,” Journal of Thoracic and Cardiovascular Surgery 89(4): 531-546; Magovern, G. J. Jr., Flaherty, J. T., Gott. V, L., Bulkley, B. H., and Gardner, T. J. 1982. “Failure of Blood Cardioplegia to Protect Myocardium at Lower Temperatures,” Circulation 66(2 Part 2): 160-167).

The challenges and complications associated with single lung ventilation warrant the use of an alternative means for ventilation during thoracic surgery. Single lung ventilation seeks to avoid the collapse and sustained ventilation of a single lung during thoracic surgery (see, for example, Matsui, Y., Takazawa, T., Takemae, A., Murooka, Y., Kanamoto, M. and Saito, S. 2022. “Investigation of the Optimal Method of Oxygen Administration with Simultaneous Use of a Surgical Mask: A Randomized Control Study,” Online, Journal of Anesthesia 36(1): 26-31; Creech, K. T. and Chaudhry, U., 2021. “One-lung Ventilation in a Patient with Necrotizing Pneumonia Complicated by a Bronchopleural Fistula,” Online, Cureus (Palo Alto, California) 13(5): e15138; Ishida, Y., Okada. T., Kobayashi, T., and Uchino, H. 2021. “ORi™ [oxygen reserve index]: A New Indicator or Oxygenation,” Online, Journal of Anesthesia 35(5): 734-740; Mehrotra, M. and Jain, A. 2021. “Single Lung Ventilation,” Online, Treatsure Island, Florida: StatPearls Publishing Co.; Sagiroglu, G., Baysal, A., and Karamustafaoglu, Y, A. 2021. “The Use of Ocygen Reserve Index in One-lung Ventilation and Its Impact on Peripheral Oxygen Saturation, Perfusion Index, and Pleth Variability Index,” Online, BioMed Central Anesthesiology 21(1): 319; Templeton, T. W., Piccioni, F. and Chatterjee, D. 2021. “An Update on One-lung Ventilation in Children,” Online, Anesthesia and Analgesia 132(5): 1389-1399; Marongiu, I., Spinelli, E., and Mauri, T. 2020. “Cardio-respiratory Physiology during One-lung entilation: Complex Interactions in Need of Advanced Monitoing,” Online, Annals of Translational Medicine 8(8): 524; Berg. S., Bittner, E. A., Berra, L., Kacmarek, R. M., and Sonny, A. 2019. “Independent Lung Ventilation: Implementation Strategies and Review of Literature,” Online, World Journal of Critical Care Medicine 8(4): 49-58; Zheng. M., Niu. Z., Chen, P., Feng, D. Wang, L., and 4 others 2019. “Effects of Bronchial Blockers on One-lung Ventilation in General Anesthesia: A Radnomized Controlled Trial,” Medicine (Baltimore, Maryland) 98(41): e17387; Ashok. V. and Francis, J. 2018. “A Practical Approach to Adult One-lung Ventilation,” Online, BJA [British Journal of Anaesthesia] Education 18(3): 69-74; Liu, Z., Liu X., Huang. Y., and Zhao, J. 2016. “Intraoperative Mechanical Ventilation Strategies in Patients Undergoing One-lung Ventilation: A Meta-analysis,” Online, Springerplus 5(1): 1251; Pandharikar, N. Sachdev, A., Gupta, N., Gupta, S. and Gupta, D. 2016. “Chest Trauma: A Case for Single Lung Ventilation,” Online, Indian Journal of Critical Care Medicine 20(4): 248-250; David. P., Pompeo, E., Fabbi, E., and Dauri, M. 2015, Op cit., this section; Fabila, T. S. and Menghraj, S. J. 2013. “One Lung Venilation Strategies for infants and Children Unddergoing Video Assisted Thoracoscopic Surgery,” Online, Indian Journal of Anesthesiology 57(4): 339-344; Kaza, S. R., Maddali, M. M., Albahrani, M. J. and Vaghari, A. A., 2012. “One Lung Ventilation in a Patient with an Upper and Lower Airway Abnormality,” Online, Indian Journal of Anaesthesia 56(6): 567-569; Gong, Q., Yang, Z., and Wei, W. 2010. “The Changes of Pulmonary Blood Flow in Non-ventilated Lung during One Lung Ventilation” Journal of Clinical Monitoring and Computing 24(6): 407-412; Cohen, E. 1997. “Physiology of the Lateral Position and One-lung Ventilation,” Chest Surgery Clinics of North America 7(4): 753-771), and nonintubayed selective lobar collapse (see, for example, Zheng, H., Hu, X. F., Jiang, G. N., Ding, J. A., and Zhu, Y. M. 2017. “Nonintubated-awake Anesthesia for Uniportal Video-assisted Thoracic Surgery Procedures,” Thoracic Surgery Clinics 27(4): 399-406 Sihoe, A. D., Ho, K. M., Sze. T. S., Lee. T. W., Yim, A. P. 2004. “Selective Lobar Collapse for Video-assisted Thoracic Surgery,” Annals of Thoracic Surgery 77(1): 278-283), are the two methods used to avoid deflation of lung tissue other than that operated on by minimally invasive techniques to include endovascular, endoscopic, robotic, and video-assisted.

### Complications of One-Lung Ventilation

“Both lungs may be affected independently by single lung ventilation. The ventilated dependent lung is prone to ventilator-induced lung injiry due to higher tidal volumes used. The non-dependent non-ventilated lung is prone to injury by surgical trauma and ischemia-reperfusion injuries,” (see, for example, Colquhoun, D. A., Leis, A. M., Shanks, A. M., Mathis, M. R., Naik, B. I., and 13 others 2021. “A Lower Tidal Volume Regimen during One-lung Ventilation for Lung Resection Surgery is Not Associated with Reduced Postopeative Pulmonary Complications,” Online, Anesthesiology 134(4): 562-576; Mehrotra, M. and Jain, A. 2021. “Single Lung Ventilation,” Online, Treatsure Island, Florida: StatPearls Publishing Co., Op cit. above in this section) (see, also, for example, Lin. M., Shen, Y., Feng. M., and Tan, L. 2019. “Is Two Lung Ventilation with Artificial Pneumothorax a Beter Choice Than One Lung Ventilatio in Minimally Invasive Esophagecomy?” Online, Journal of Thoracic Disease 11 (Supplement5): S707-S712; Aloud, A., Berdine, G., and Nugent, K. 2018. “Single Lung Ventilation in Patients Undergoing Lobectomy,” Online, Journal of Thoracic Disease 10(12): 6383-6387; Colquhoun, D. A., Naik, B. I., Durieux, M. E., Shanks, A. M., Kheterpal, S., Bender, S. P., and Blank, R. S. 2018. “Management of One-lung Ventilation-Variation and Trends in Clinical Practice: A Report fro the Multicenter Perioperative Outcomes Group (MPOG),” Online, Anesthesia and Analgesia 126(2): 495-502; Grande, B. and Ganter, M. T. 2018. “What is the Best Strategy for One-lung Ventilation during Thoracic Surgery:” Online, Journal of Thoracic Disease 10(12): 6404-6406; Blank, R. S. Colquhoun, D. A., Durieux, M. E., Kozower, B. D., McMurry, T, L., Bender, S. P., and Nalk, B. I., 2016. “Management of One-lung Ventilation: Impact of Tidal Volume on Complications after Thoracic Surgery,” Online, Anesthesiology 124(6): 1286-1295; Roth, J. V., 2016. “Complications of One-lung Ventilation: Is It the Blood Flow or the Ventilation?,” Online, Anesthesiology 126(6): 1253-1254; Purohit, A., Bhargava, S., Mangal. V., and Parashar, V. K. 2015. “Lung Isolation, One-lung Ventilation and Hypoxaemia during Lung Isolation,” Indian Journal of Anaesthesia 59(9): 606-617).

Moreover, one-lung, or single lung, ventilation is inadequate for sustaining lung inflation with or without the patient on a ventilator when undergoing a bilateral clamshell thoracotomy for radical cardiac surgery where general anesthesia, bypass, cross-clamping, cardioplegia, and so on would best be avoided and regional anesthesia used instead. Provided the problem of iatrogenic pneumothorax could be avoided, extracardiac, a switch heart transplant could be performed under regional rather than general anesthesia, thus avoiding the need for cardiopulmonary bypass, cross-clamping, cardioplegia, the risk of postperfusion syndrome, and so on. To perform an operation under general anesthesia and incur the risks associated therewith when the only reason for doing so is to avoid lung collapse is avoided through the use, briefly described above, of a surgical chest dome designed for thoracic surgery.

Contrary to the situation with conventional heterotopic heart transplantation, double heart transplantation using the metered switch technique comprehends in vivo support measures that allow the deliberate selection of a smaller donor heart. When the native heart is not in end stage failure, the added ejection force of a smaller heart with lower right ventricular ejection fraction, or absolute stroke volume, is less likely to result in pulmonary hypertension. To compensate for the temporary reduction in alveoli, the reduced lung will develop additional alveoli over the ensuing year to eradicate any dyspnea the patient would otherwise experience, and any cough and/or gastroesophageal reflux should subside.

The volume of lung removed is not so large that alveolar regeneration should require the use of an incentive spirometer or a special exercise routine for the purpose. Within a year, breathing even during rigorous exercise, should be little if at all affected. When necessary, alternative locations for the second heart are addressed below under 2b. Background of the Invention, Vascular Valves and Servovalves in Vascular Diversion, (1). Intercorporeal Diversion of Blood Flow, (b). Metered Compound vascular bypass, or sudden switched-organ Transplantation, 1b(1)(b)(b5). Double Heart Metered Compound vascular bypass, or sudden switched-organ Transplantation, but the chest is preferred.

The cardiotoxicity of chemotherapy and radiation used to treat a malignancy addressed below in this section is typical of the benefit to be gained from the capability to automatically target medication directly to an organ with ancillary means for preventing the exposure of nontargeted tissue to the medication is backward compatible with to retroactively increase the efficacy of all previous pharmacy and surgery. Methods of treatment since discounted, such as the Vineberg procedure, may warrant reevaluation in light of the later advent of such means. In solid organ transplantation, the implanted control system administers the transition from the circulatory system of the donor into that of the recipient as well as continues to target medication or electrostimulation to the graft organ.

Such medical surgery, or surgery to position catheteric piping and connectors to allow the direct targeting of organs or tissues through the blood supply or directly into the parenchyma is undertaken endoscopically through “keyhole” incisions that readily heal. The vessels, such as the renal or hepatic arteries, and structures, such as the urinary bladder, are usually large and easily located without the need for contrast dye. The implanted microcontroller or microprocessor and small flat drug reservoirs are usually positioned in the pectoral region and hardly noticeable from a cosmetic standpoint

Turning now to the automated local, or topical, release of radioisotopes internally in lieu of brachytherapy or external beam teletherapy, a natural nutrient at normal levels, iodine is harmless to nonthyroidal tissue. However, the use of iodine¹³¹ to treat toxic goiter or thyroid carcinoma does expose nontargeted tissue to radiation. The systemic, or indiscriminate dissemination, of chemotherapeutics, antiangiogenics, and steroids, much less when these are delivered in radioactive form-even though the dosage level optimal for the actual target is limited by systemic distribution—is always harmful.

Long-term consequences of irradiation may not appear for many years following such therapy. No less significantly, nontargeted delivery, essentially indiscriminate systemic dispersal, of potentially harmful drugs limits the dosage levels which can be delivered to the target. Nevertheless, where a malignancy has disseminated, or metastasized, systemic delivery cannot be avoided. The ability to bypass bystander tissue and directly aim the agent at the target tissue is fundamentally more effective and less problematic.

Dispersal thus is both harmful to nontargeted tissue, and beause the dose must be reduced, the efficacy for targeted tissue is also reduced (“High-dose chemotherapy regimens are predicated on the observation that the concentration-effect curve for many anticancer agents is rather steep, and increased dose can produce markedly increased therapeutic effect, although at the cost of potentially life-threatening complications that require intensive support, usually in the form of hematopoietic stem cell support from the patient (autologous) or from donors matched for histocompatibility loci (allogeneic,”) Sausville, E, A. and Longo, D. L. 2005. “Principles of Cancer Treatment: Surgery, Chemotherapy, and Biologic Therapy,” in Kasper, D. L, Braunwald, E., Fauci, A. S., Hauser, S, L., Longo, D, L., and Jameson, J. L. Harrison's Principles of Internal Medicine, Chapter 70, page 467, New York, New York: McGraw-Hill Medical Publishing Division).

When the components of the disorder control system used to internally convey and deliver the drugs are radiation shielded, the drugs can include chemotherapeutic-free radioactive liquids, fluid radioactive chemotherapeutics, antiangiogenics, gene therapeutics, and steroids, for example. Where micrometastases have been ruled out, the benefit in locally targeted over systemic application is clear. Radiolabeled chemotherapeutics have been used for diagnostic purposes but local, topical, rather than systemic use will require direct pipeline-targeting (see, for example, Papachristou, M., Kastis, G. A., Stavrou, P. Z., Xanthopoulos, S., Furenlid, L. R., Datseris, I. E., and Bouziotis, P. 2018. “Radiolabeled Methotrexate as a Diagnostic Agent of Inflammatory Target Sites: A Proof-of-Concept Study,” Molecular Medicine Reports 17(2): 2442-2448).

The unavoidable adverse side effects with cancer fighting drugs administered systemically to kill any undetectable micrometastases may allow a reduction in frequency if not dose when the primary neoplasm is directly pipeline-targeted at a higher frequency. Radiation shielded fluid drug delivery lines in a direct pipeline-targeting ambulatory disorder response system permit the use of not only ordinary but radioactive chemotherapeutics, healthy stem cells, antiangiogenics, and biologics, to include gene, such as tumor and metastasis suppressor gene, caretaker gene, immunosuppressive therapy, and biological, such as hormonal and autogenous or allogeneic stem cell therapy, for example.

[see p. 98 Use of Radiation]

Referring now to the introductory section above entitled Use of Radiation, FIG. 14A depicts one therapeutic technique which depends upon vascular servovalves, in this case, a servochoke shown in FIGS. 14A and 14B, to continuously adjust the level of hypoxia of unaffected tissue surrounding a tumor to that optimal for reducing its radiosensitivity, hence, susceptibility to damage due to spillover. In FIGS. 14B and 14C, both the drip probe at the upper left of the kidney and servochoke on the renal artery can have multiple accessory channels, each capable of delivering one or more chemo- or radioactive therapeutic drip agents.

The latter are best selected with a biological half-life and low emission dose rate that avoid the need for recovery with radionuclides such as gamma ray-emitting palladium 103 with a biological half-life of 17.0 days at, on average, 21 kiloelectron volts. However, if necessary, recovery is possible through, for example, bonding the radionuclide or radioisotope to a superparamagnetic carrier thereby rendered recoverable with the aid of a magnetized impasse-jacket, of which one type is shown and illustrated in U.S. Pat. No. 11,389,171 entitled Integrated System for the Infixion and Retrieval of Implants. While conventional impasse-jackets would require reentry to be recovered, these too, much as might the polymeric components of other type jackets and valves, can be made of absorbable materials. U.S. Pat. No. 11,389,171 also addressed absorbable materials in relation to radiation shielding, in section 8b(2)(a)(viii) et sequentia, for example.

### Focused Drip Brachyradiochemotherapy

The object in avoiding surgical excision is to significantly reduce the trauma of removal, which in an intricate organ such as the kidney illustrated can fundamentally degrade the outcome of treatment. An alternative method for inducing hypoxia, one that does not result in an increase in upstream blood pressure, is to divert a portion of the blood supplied to the affected organ to a large vessel nearby. These examples illustrate the direct relevance to vascular valves and servovalves of focused drip brachytherapy. Means for implementing implanted automatically controlled directly focused treatment of tumors is shown and described in U.S. Pat. No. 11,013,858, entitled Nonjacketing Side-entry Connectors and Prosthetic Disorder Response Systems, FIGS. 13A thru 16 (Harris, A. L. 2002. “Hypoxia-A Key Regulatory Factor in Tumour Growth,” Nature Reviews. Cancer (London, England) 2(1): 38-47).

This basic scheme can be combined with means prompted by the same underlying concept (see, for example, Zhang, H., Montesdeoca, N., Tang, D., Liang, G., Cui, M., and 8 others 2024. “Tumor-targeted Glutathione Oxidation Catalysis with Ruthenium Nanoreactors against Hypoxic Osteosarcoma,” Nature Communications (London, England) 15(1): 9405; Cole H D, Roque J A 3rd, Shi G, Lifshits L M, Ramasamy E, and 4 others 2022. “Anticancer Agent with Inexplicable Potency in Extreme Hypoxia: Characterizing a Light-triggered Ruthenium Ubertoxin,” Online, Journal of the American Chemical Society (Washington, D.C.) 144(22): 9543-9547).

Focused drip brachytherapy also requires one or more drug storage reservoirs, usually implanted subcutaneously in the pectoral region, with a drip release or stopcock device which can be controlled by the implanted microcontroller in monomorbid disease or control microprocessor in comorbid disease. At less highly radioactive levels or lower dose rates, the polymeric material comprising the components along the route may provide sufficient shielding to protect neighboring structures. Otherwise, all must be shielded for the level of radioactivity used. Advancement of the drip releasing probe into the tumor and coordinated action, such as the release through one or more accessory channels of sulfhydryl compounds, for example, is automatically controlled by the implanted controller on the basis of sensor feedback indicating the instant condition of the mass.

Indicia indicative of a malignancy suitable or adaptable for detection by implanted sensors-positioned at the root, or site, of disease or to detect biomarkers in the passing blood-range from the simple, such as a temperature differential, to the detection of chemical analytes characteristic of a malignant process (see, for example, El-Khoury, V., Schritz, A., Kim, S. Y., Lesur, A., Sertamo, K., and 18 others 2020. “Identification of a Blood-based Protein Biomarker Panel for Lung Cancer Detection,” Cancers (Basel, Switzerland) 12(6): 1629; Gutierrez-Capitan, M., Baldi, A., and Fernández-Sánchez, C. 2020. “Electrochemical Paper-based Biosensor Devices for Rapid Detection of Biomarkers,” Sensors (Basel, Switzerland) 20(4): 967; Li, J., Guan, X., Fan, Z., Ching, L.-M., Li, Y., and 3 others 2020. “Non-invasive Biomarkers for Early Detection of Breast Cancer,” Cancers (Basel, Switzerland) 12(10): 2767; Loo, S. W. and Pui, T.-S. 2020. “Cytokine and Cancer Biomarkers Detection: The Dawn of Electrochemical Paper-based Biosensor,” Sensors (Basel, Switzerland) 20(7): 1854; Ahmad, O. S., Bedwell, T. S., Esen, C., Garcia-Cruz, A., and Piletsky, S. A. 2019. “Molecularly Imprinted Polymers in Electrochemical and Optical Sensors,” Trends in Biotechnology 7(3): 294-309; Sadri, B., Goswami, D., de Medeiros, M. S., Pal, A., Castro, B., Kuang, S., and Martinez, R. V. 2018. “Wearable and Implantable Epidermal Paper-based Electronics,” American Chemical Society Applied Materials and Interfaces 10(37): 31061-31068).

Moreover, many therapeutic radiopharmaceuticals are usable independently or in combination with others or imaging radiopharmaceuticals to not only destroy malignant tissue but to allow imaging to support expedient examination of the status in tumor destruction as treatment proceeds (see, for example, Rangger, C. and Haubner, R. 2020. “Radiolabelled Peptides for Positron Emission Tomography and Endoradiotherapy in Oncology,” MDPI [Multidisciplinary Digital Publishing Institute] Pharmaceuticals (Basel, Switzerland) 13(2): 22; Young, R. P., Christmas, T., and Hopkins, R. J. 2018. “Multi-analyte Assays and Early Detection of Common Cancers,” Journal of Thoracic Disease (Hong Kong, China) 10 (Supplement 18): S2165-S2167; Ferreira, C. de A., Fuscaldi, L. L, Townsend, D. M., Rubello, D., and Branco de Barros, A. L. 2017. “Radiolabeled Bombesin Derivatives for Preclinical Oncological Imaging,” Biomedicine and Pharmacotherapy 87:58-72; Gutfilen B. and Valentini, G. 2014. “Radiopharmaceuticals in Nuclear Medicine: Recent Developments for SPECT and PET Studies,” Online, Biomed Research International 2014:426892).

Radioactive shielding is described and illustrated in FIGS. 10A and 10B of U.S. Pat. No. 11,013,858, entitled Nonjacketing Side-entry Connectors and Prosthetic Disorder Response Systems. The drug reservoir and delivery components. Unlike sequentially administered, or fractionated, radioactive wire brachytherapy or external beam radiation wherewith entry and removal require that patency to the body exterior be maintained or reestablished, once implanted, delivery of a radioactive drip is interrupted or stopped without access to the target volume.

Rather, once totally implanted, dosing is controlled by the automatic disorder response control system microcontroller without reentry in accordance with its prescription-program. The progress in treatment is reported to the adaptively programmed microcontroller by temperature, oxygen level, and chemical sensors and can be viewed noninvasively by imaging. Throughout treatment, the radioactive drip confines radiation to within the tumor or target volume. The regimen, that is, the frequency and the dose, for radiation treatment is established on the basis of initial imaging.

A second delivery line can be used to position metastasis suppressor genes for example, in contiguous relation to a potentially primary or ‘mother’ tumor to suppress the metastatic process with the shedding of ‘daughter’ cells and the development of disseminated micrometastases. Averting this eventuality would then limit the effectiveness of tumor resection and demand the delivery of chemotherapeutics systemically with its serious side effects. That the drug is targeted does not mean that a background dose cannot be provided to treat any micrometastases.

The infusion of higher therapeutic dose radiactive substances to pass through the systemic circulation would inevitably result in endothelial and/or intimal senility and eventually, vasculopathy. By substantially reducing if not eliminating the side effects of systemic delivery, the directly pipeline-targeted local rather than systemic application of chemotherapeutics eliminates the need to avoid of a drug or drugs that tightly targeted would be optimal.

More generally, in avoiding nontargeted tissue, the direct pipeline-targeting of drugs and radioactive agents in fluid form directly to the root, or site, of disease, whether an organ, circumscribed volume of tissue, or a malignant original root-site of disease, allows dosing responsive exclusively to the therapeutic requirement without regard, much less restraint in dosing, due to reservations about the potential for provoking adverse side effects elsewhere in the body. Such applications are not without caveats-prodrugs, for example, or agents activated by passing through the liver must first be converted into their form following processing by the liver to function when directly targeted for local or topical use.

Currently chemotherapeutic drugs may be used as adjuvant to radiation (concurrent chemoradiation therapy) (see, for example, Boeckman, H. J., Trego, K. S., Henkels, K. M., and Turchi, J. J. 2005. “Cisplatin Sensitizes Cancer Cells to Ionizing Radiation via Inhibition of Non-homologous End Joining,” Molecular Cancer Research 35): 277-285; Bocci, G., Nicolaou, K. C., and Kerbel, R. S. 2002. “Protracted Low-dose Effects on Human Endothelial Cell Proliferation and Survival in Vitro Reveal a Selective Antiangiogenic Window for Various Chemotherapeutic Drugs,” Cancer Research 62(23): 6938-6943; Klement, G., Baruchel, S., Rak, J., Man, S., Clark, K., and 3 others 2000. “Continuous Low-dose Therapy with Vinblastine and VEGF Receptor-2 Antibody Induces Sustained Tumor Regression without Overt Toxicity,” Journal of Clinical Investigation 105(8): R15-R24). However, the lack of delivery means for direct, or topical, application of radioactive chemotherapeutics, hence, the potential for application, affords no incentive for the development of such substances. For local application, drugs ordinarily delivered systemically which require passage through the liver to be activated require to be converted into the post-liver active form before administration.

Radiation able both to initiate malignant degeneration and kill established cancer, the direct targeting of an adjuvant or concomitant chemotherapeutic rather than its systemic dispersal through infusion, for example, can be used to avoid the sensitization of neighboring tissue at the same time that it increases the sensitization of the tumor or target tissue. Tight targeting of the adjuvant and radiation both separately and together are equally important. Delivery of either through separate accessory channels to the same nonjacketing side-entry connector or ductus side-entry jacket, for example, allows their coordinated targeted arrival.

With brachytherapy, the source of radiation is positioned beside or within the target volume, and with teletherapy, or external beam radiation, it is aimed at the target volume. Neither allows automatically scheduled fractionated delivery away from the clinic. By the same token, conventional beam radiation and brachytherapeutic ‘seeds’ implanted within the affected organ, allow the radiation to be stopped by removing the source. However, both are confined to the clinic, and the removal of seeds if necessary is tedious.

In contrast, when dispensed in the form of an automatically controlled radioisotope, or radionuclide, drip, only so much of the radioactive agent is released into the tumor at a time as incidence with the malignant tissue of the tumor will expend in the destructive reorganization of the target DNA. Controlled by an implanted microcontroller responsive to implanted sensor data concerning temperature, oxygen level, and chemical analytes appurtenant of a malignant tumor, the drip rate can be decreased, increased, stopped, or restarted at any time, the patient ambulatory, going about his business, and oblivious to the process.

The direct pipeline-targeting of therapeutic radiation as a drip allows minimizing if not eliminating the exposure of surrounding nontargeted tissue, or spillover (“bystander effect,” abscopal exposure) encountered in the use of alternative forms of ionizing radiation as a perpetual problem in their use. The levels of radiation pertinent here are therapeutic, not just radiological for imaging or for radiolabel tracing, for which the material of the catheters used as the mainlines or accessory channels of ductus side-entry jackets, nonjacketing side-entry connectors, valve-jackets, and servochokes can provide adequate shielding without the need for layering tungsten, for example, about the outer surface of the catheter used as the mainline or accessory channel (sideline).

With direct pipeline-targeting, the chemotherapeutic and radioactive substance can be delivered separately or together, either in any sequence and dose, with acquisition of the target by direct contact, not at a distance as would allow spillover. Separate chemotherapeutic and radiation lines can deliver chemotherapeutic, radioactive, antimicrobial, immunosuprressive, or anti-inflammatory agents in any sequence at timed intervals, or, if both lines are shielded, both can deliver the same agents. The inability to focus radiation on the tumor by the current methods of brachytherapy and external beam radiation, dose-sculpting treatment techniques aside, often results in serious complications that demand follow-up surgical procedures, and these in turn often result in secondary complications which demand surgical remediation.

For example, the sensitization to ionizing radiation induced by neoadjuvant chemotherapy aside, in the attempt to eradicate pelvic tumors, radiation spillover and toxicity have proven especially problematic in inducing radiation enteritis, often obliterative, and/or radiation proctitis, of which the long-term complications require surgical correction in turn susceptible to complications, severely eroding patient quality of life (see, for example, Cai, Y., Ma. T., Zhong, Q., Qin, Q. Cao. W., Liu, Z., Ke. J., and Wang, H. 2020. “Quantitative C T Measurement of Left Colonic and Pelvic Mesenteric Adipose Volume in Radiation Proctitis,” Annals of Translational Medicine 8(14): 882; Chen, L, Liao, F., Jiang, Z., Zhang, C. Wang, Z., and 16 others 2020. “Metformin Mitigates Gastrointestinal Radiotoxicity and Radiosensitises P53 Mutation Colorectal Tumours via Optimising Autophagy,” British Journal of Pharmacology 177(17): 3991-4006; Hoffmann, M., Waller, K., Last. A., and Westhuyzen, J. 2020. “A Critical Literature Review on the Use of Bellyboard Devices to Control Small Bowel Dose for Pelvic Radiotherapy,” Reports of Practical Oncology and Radiotherapy 25(4): 598-605; Qian, L. and Cen, J. 2020. “Hematopoietic Stem Cells and Mesenchymal Stromal Cells in Acute Radiation Syndrome,” Oxidative Medicine and Cellular Longevity 2020:8340756. Salah, M., Osuga, S., Nakahana, N., Irino, Y., Shinohara, M., and 9 others 2020. “Elucidation of Gastrointestinal Dysfunction in Response to Irradiation Using Metabolomics,” Biochemistry and Biophysics Reports, Online at reader.elsevier.com/reader/sd/pii/S2405580820300984?token=2C77274 BBDA270FE667238DEFA37D4956602550D1B832E208CEF0A80510250DC15869E4C70A2FA69F6D586638A3CC54A; Paleny, R., Bremer, M., Walacides, D., Mainwaring, S., Weber. K., and Henkenberens, C. 2018. “Comparison of Relative and Absolute Rectal Dose-volume Parameters and Clinical Correlation with Acute and Late Radiation Proctitis in Prostate Cancer Patients,” Strahlentherapie und Onkologie [Radiotherapy and Oncology] 195(2): 103-112; Moussa, L., Pattappa, G., Doix, B., Benselama. S.-L., Demarquay, C., and 7 others 2017. “A Biomaterial-assisted Mesenchymal Stromal Cell Therapy Alleviates Colonic Radiation-induced Damage,” Biomaterials 115:40-52; Zhong, Q. H., Wu, P. H., Qin, Q. Y., Kuang, Y. Y., Ma, T. H., Wang, H. M., Zhu, Y. X., Chen, D. C., Wang, J. P., and Wang. L. 2017. “Pathological Insights of Radiotherapy-related Damage to Surgical Margin after Preoperative Radiotherapy in Patients with Rectal Cancer,” (in Chinese with English abstract at Pubmed), Zhonghua Wai Ke Za Zhi [Chinese Journal of Surgery] (Beijing, China] 55(7): 507-514; Moussa, L., Usunier, B., Demarquay, C., Benderitter, M., Tamarat. R., Semont. A., and Mathieu, A. 2016. “Bowel Radiation Injury: Complexity of the Pathophysiology and Promises of Cell and Tissue Engineering,” Cell Transplantation 25(10): 1723-1746; Brenner, H. Kloor. M., and Pox, C. P. 2014. “Colorectal Cancer,” Lancet 383:1490-1502; Hauer-Jensen, M., Denham, J. W., and Andreyev, H J. N. 2014. “Radiation Enteropathy—Pathogenesis, Treatment, and Prevention,” Nature Reviews. Gastroenterology and Hepatology 11(8): 470-479; Stacey, R., and Green, J. T. 2014. “Radiation-induced Small Bowel Disease: Latest Developments and Clinical Guidance,” Therapeutic Advances in Chronic Disease 5(1): 15-29; Mahmoud, N N. And Fry, R, D. 2008. “Radiation Injury to the Small and Large Bowel,” in Cameron, J. L. (ed.), Current Surgical Thherapy, Philadelphia, Pennsylvania: Mosby/Elserier, pages 205-209).

By contrast, the direct pipeline-targeting of ionizing radiation to malignant tissue, constrains DNA destruction to the tumor without spillover to adjacent healthy tissue. In most instances, the advantage of avoiding a preliminary requirement to establish a track for channeling the source of ionizing radiation directly to the target by implantation does not begin to offset the potential harm associated with the lack of containment. For this reason, direct topical irradiation is therefore a significant capability.

That the benefit gained from chemotherapeutics and steroids would be significantly amplified were untargeted tissue bypassed is clear. While higher dose rate agents should not be allowed into contact with normal endothelium in the young not otherwise facing imminent death, still greater benefit will be had from these and other drugs when radioactive and directly pipeline-targeted at dose rates higher than tracer levels. Due to the lack of means for providing adequate protection for neighboring tissue, the development of radioactive fluids for direct delivery into the interior of malignant tissue as a radioactive drip by a totally implanted system has gone unexplored. U.S. Pat. No. 11,013,858, entitled Nonjacketing Side-entry Connectors and Prosthetic Disorder Response Systems, illustrates and describes absorbable and permanent radiation shielding for tissue connectors.

When more than a moderate dose rate is involved, the need for shielding applies to every component along the drug delivery path, to include the drug reservoirs, reservoir outlet pumps or drip stopcocks, druglines leading from the reservoir outlet to the targeted connector, as well as the targeted connector itself. The radiactive source is delivered as a drip wherein the dose per drop assures that its radioactivity will be expended before the next drop is released. Depleted of its capacity to irradiate, there is no need to prevent a residue from the targeted site, organ, or gland from entering the circulation and affecting the endothelia or other nontargeted tissue. An implanted scheme for the automatic dispensing of a radioisotope must not allow the exposure to radiation of endothelia lining the venous drainage and the healthy tissue surrounding the tumor, healthy tissue more highly oxygenated and therefore more susceptible to injury from radiation

Some disagreement having appeared in the literature as concerns environmental and non-oncological exposure to radiation notwithstanding (see, for example, Cardarelli, J. J. II and Ulsh, B. A. 2018. “It is Time to Move Beyond the Linear No-threshold Theory for Low-Dose Radiation Protection,” Dose-Response 16(3): 1559325818779651; Doss M. 2018. “Comment on '30 Years Follow-up and Increased Risks of Breast Cancer and Leukaemia after Long-term Low-dose-rate Radiation Exposure,” British Journal of Cancer 118(5): e9; Siegel, J. A., Pennington, C. W., Sacks, B., and Welsh, J. S. 2018. “The Birth of the Illegitimate Linear No-threshold Model: An Invalid Paradigm for Estimating Risk Following Low-dose Radiation Exposure,” American Journal of Clinical Oncology 41(2): 173-177; Hsieh, W. H., Lin, I. F., Ho, J. C., and Chang, P. W. 2017. “30 Years Follow-up and Increased Risks of Breast Cancer and Leukaemia after Long-term Low-dose-rate Radiation Exposure,” British Journal of Cancer 117(12): 1883-1887), from the standpoint of therapeutic and not just radiological dose levels, no minimum level of exposure to ionizing radiation, especially of tissues with a high rate of cell turnover such as type B spermatogonia, endothelial linings of vessels and other ductus, to include the urothelial lining the urinary tract has been established as safe.

Directly pipeline-targeted chemotherapy in liquid form delivered directly into the target volume through synthetic lines is preferable to conventional forms of radiotherapy, which often lead to late-term vascular senility and vasculopathy. Tumors induced in nontargeted tissue having resulted from multiple exposure to ionizing radiation may have adapted to resist the killing of their cells “ . . . through cycled accumulation of genetic mutations and selection for increased survival and proliferation,” (Reisz, J. A., Bansal, N., Qian, J., Zhao, W., and Furdui, C. M. 2014. “Effects of Ionizing Radiation on Biological Molecules-Mechanisms of Damage and Emerging Methods of Detection,” Antioxidants and Redox Signaling (Larchmont, New York) 21(2): 260-292).

The next drop is not released until the drop preceding it is largely if not completely depleted, so that there is no need to intercept or recover the fluid in the venous outflow. Where depletion is incomplete so that some slight residue drains into the organ vein, clinical judgement would usually consider the benefit worth the risk. If not, a particulate source of radiation, alpha or beta radiation, is neutralized by binding to a magnetically susceptible radiation tolerant carrier (see, for example, King, A. P., Lin, F. I., and Escorcia, F. E., 2021. “Why Bother with Alpha Particles,” European Journal of Nuclear Medicine and Melocular Imaging 49(1): 7-17; Napoli, E., Bonsdorff, T. B., Jorstrad, I. S., Bruland, O. S., Larsen, R. H., and Westrom, S. 2021. “Radon-220 Diffusion from 224Ra-labeled Calcium Carbonate Microparticles: Some Implications for Radiotherapeutic Use,” Online, PLoS [Public Library of Science] One 16(3): e0248133; Li, M., Sagastume, E. A., Lee, D., McAlister, D., DeGaffenreid, and 8 others 2020. “^{203/212}Pb Theranostic Radiopharmaceuticals for Image-guided Radionuclide Therapy for Cancer,” Current Medicinal Chemistry 27(41): 7003- 7031; Reissig, F., Zarschler, K., Hubner, R., Pietzsch, H.-J., Kopka. K., and Mamat, C. 2020. “Sub-10 nm Radiolabeled Barium Sulfate Nanoparticles as Carriers for Theranostie Applications and Targeted Alpha Therapy,” ChemistryOpen 9(8): 797-805; McDevitt, M. R., Sgouros, G., and Sofou, S. 2018. “Targeted and Nontargeted α-Particle Therapies,” Annual Review of Biomedical Engineering 20:73-93. Sgouros, G., Hobbs, R., and Josefsson, A. 2018. “Dosimetry and Radiobiology of Alpha-particle Emitting Radionuclides,” Current Radiopharmaceuticals 11(3): 209- 214; Tafreshi, N. K., Doligalski, M, L., Tichacek, C. J., Pandya, D. N., Budzeivich, M M., and 6 others 2018. “Development of Targeted Alpha Particle Therapy for Solid Tumors,” Online, Molecules (Basel, Switzerland) MDPI [Multidisciplinary Digital Publishing Institute] Basel, Switzerland( ) 24(23): 4314; references pertaining to decreasing the susceptibility of healthy tissue surrounding a solid tumor to radiation through the inducement of hypoxia greater than that of the tumor using a servochoke cited above in this section).

At lower doses, its emission is contained within the reservoir without the need for radiation shielding. At lower dose rates, emission is contained by the ordinarily polymeric materials of each component along the delivery route; at higher dose rates, radiation shielding is applied to each component comprising the route, to include the entry port at the body surface, reservoir, drip mechanism, and nonjacketing side-entry connector.

Adverse sequelae often not appearing until decades after exposure to radiation, its avoidance is important in direct proportion to the youth and inverse proportion to the age of the patient. Endothelia acutely sensitive to ionizing radiation, passing a radioactive drug through a side-entry jacket into the blood supply of an organ containing a malignant tumor is to be strongly discouraged in the young. However, in those near death or old enough that the late effects of radiation exposure will not have had sufficient time to appear before the patient, otherwise left in intractable pain, dies, the analgesic benefit of such use should not be perfunctorily discounted.

In younger patients, radiation should be avoided or a shielded and magnetized impasse-jacket provided. Incorporating a magnetized layer in side-entry jackets, valves, and connectors to repel a superparamagnetic iron oxide carrier-bound radioactive micro- or nanoparticulate in a liquid medium from contact with the endothelial or urothelial lining of the ductus would afford some protection but only coextensive with the interior of the device; such a perivascular surround would have to be continued to the endothelium beyond the jacket to provide the protection needed.

Especially in the young in whom late-term sequelae of radiation could emerge decades after treatment, the healthy vasculature is avoided by inserting the distal end of the radioactive drip delivery mainline catheter directly into the tumor rather than by infusion into the main supply artery through a side-entry jacket. Release of the radioactive agent directly into the interior of the tumor with a radiation track range that substantially restricts irradiation to within the tumor eliminates the exposure of 1. Superjacent tissue as a transit volume, 2. Healthy tissue surrounding the tumor, and at lower dose rates. 3. The drainage endothelium.

Direct pipeline-targeting allows the use of more highly radioactive radioisotopes or radionuclides alone or in combination with nonradioactive or radioactive chemotherapeutics, that is, as jointly delivered radiochemotherapy. Radioactive agents move through shielded lines while nonradioactive agents move through unshielded lines or in combination with one or more chemotherapeutics, both isolated from nontargeted tissue. Drugs such as chemotherapeutics that would be altered if irradiated can be protected with lines shielded to protect the interior flow rather than radiation from without.

Given that conventional radiochemotherapy employs targeted, or direct external beam, radiotherapy combined with systemically delivered chemotherapy, this is novel (see, for example, Gray, J. E., Villegas. A., Daniel, D., Vicente, D., Murakami, S., and 15 others 2020. “Three-Year Overall Survival with Durvalumab after Chemoradiotherapy in Stage III NSCLC-Update from PACIFIC [arbitrary name],” Journal of Thoracic Oncology 15(2): 288-293; Kasmann, L., Eze. C., Taugner, J., Roengvoraphoj, O., Dantes, M., and 4 others 2020. “Chemoradioimmunotherapy of Inoperable Stage III Non-small Cell Lung Cancer: Immunological Rationale and Current Clinical Trials Establishing a Novel Multimodal Strategy,” Radiation Oncology 15:167; Lyu. M., Shen, Y., Beharee, N., Lu, J., Deng. F., and Wang, J. 2020. “The Combined Use of Chemotherapy and Radiotherapy with PD-1 Inhibitor, Pembrolizumab [an immunotherapeutic], in Advanced Cervical Cancer: A Case Report,” Onco Targets and Therapy 13:4465-4471; Wang, B. C., Xiao, B. Y., Lin, G. H., Wang, C., and Liu, Q. 2020. “The Efficacy and Safety of Induction Chemotherapy Combined with Concurrent Chemoradiotherapy versus Concurrent Chemoradiotherapy Alone in Nasopharyngeal Carcinoma Patients: A Systematic Review and Meta-analysis,” Online, BioMed Central Cancer 20(1): 393; Kasmann, L., Taugner, J., and Manapov, F. 2019. “Chemo-/Immuno-/Radiotherapy Combination in Treatment of Solid Cancer,” Oncotarget 10(52): 5387-5388; Yang, Q., Cao, S. M., Guo, L., Hua, Y. J., Huang. P. Y., and 24 others 2019. “Induction Chemotherapy followed by Concurrent Chemoradiotherapy versus Concurrent Chemoradiotherapy Alone in Locoregionally Advanced Nasopharyngeal Carcinoma: Long-term Results of a Phase III Multicentre Randomised Controlled Trial,” European Journal of Cancer 119:87-96; Antonia, S., Villegas, A., Daniel, D., Vicente D., Murakami. S., and 28 others 2018. “Overall Survival with Durvalumab after Chemoradiotherapy in Stage III NSCLC [non-small-cell lung cancer],” New England Journal of Medicine 379(24): 2342-2350; Gunderson, A. J. and Young, K, H. 2018. “Exploring Optimal Sequencing of Radiation and Immunotherapy Combinations,” Advanced Radiation Oncology 3(4): 494-505. Setakomnukul, J. and Thephamongkhol, K. 2018. “Neoadjuvant Chemotherapy followed by Concurrent Chemoradiotherapy versus Concurrent Chemoradiotherapy followed by Adjuvant Chemotherapy in Locally Advanced Nasopharyngeal Carcinoma,” Online, BioMed Central Cancer 18(1): 329; Cao, S.-M., Yang, Q., Guo, L., Mai, H.-Q., 2. Mo. H.-Y., and 23 others 2017. “Neoadjuvant Chemotherapy followed by Concurrent Chemoradiotherapy versus Concurrent Chemoradiotherapy Alone in locoregionally advanced nasopharyngeal carcinoma: A phase III Multicentre Randomised Controlled Trial,” European Journal of Cancer. Clinical Trial (Oxford, England) 75:14-23; Chan, L., He, L., Zhou, B., Guan, S., Bo, M., and 6 others 2017. “Cancer- targeted Selenium Nanoparticles Sensitize Cancer Cells to Continuous γ Radiation to Achieve Synergetic Chemo-radiotherapy,” Chemistry, An Asian Journal (Weinheim, Germany) 12(23): 3053-3060; Chang, Y., He, L., Li, Z., Zeng, L., Song, Z., and 6 others 2017. “Designing Core-Shell Gold and Selenium Nanocomposites for Cancer Radiochemotherapy,” American Chemical Society Nano 11(5): 4848-4858; Shan, G.-P., Wang, B.-B., Zhang, P., Du, F.-L., and Yang, Y.-W. 2017. “Efficacy and Safety of Chemotherapy Combined with Stereotactic Radiotherapy in the Treatment of Nasopharyngeal Carcinoma,” Medical Science Monitor 23:5630-5636; Yu, B., Liu, T., Du. Y., Luo, Z., Zheng. W., and Chen, T. 2016. “X-ray-responsive Selenium Nanoparticles for Enhanced Cancer Chemo-radiotherapy,” Colloids and Surfaces, B, Biointerfaces (Amsterdam, North Holland, Netherlands) 39:180-189; He., L., Lai, H., and Chen, C. 2015. “Dual-function Nanosystem for Synergetic Cancer Chemo-/Radiotherapy through ROS [reactive oxygen species]-mediated Signaling Pathways,” Biomaterials 51:30-42; Baskar, R. Lee, K. A., Yeo, R. and Yeoh, K.-W. 2012. “Cancer and Radiation Therapy: Current Advances and Future Directions,” International Journal of Medical Sciences 9(3) 193-199).

That hypoxia imparts insensitivity to ionizing radiation (radioresistance, radioinsensitivity) has been known for over half a century (Busato, F., Khouzai, B. E., and Mognato, M. 2022. “Biological Mechanisms to Reduce Radioresistance and Increase the Efficacy of Radiotherapy: State of the Art,” Online, MDPI [Multidisciplinary Digital Publishing Institute] International Journal of Molecular Sciences (Basel, Switzerland) 23(18): 10211; Xie. Y., Han, J., Xie, K., and Gou, Q. 2022. “LncRNAs [long noncoding RNAs] as Biomarkers for Predicting Radioresistance and Survival in Cancer: A Meta-analysis,” Online, (Nature Research) Scientific Reports 12(1): 18494; Xu. Y., Tang. C., Wu. Y., Luo, L., Wang. Y., Wu. Y., and Shi, X. 2022. “Prediction of Response to Radiotherapy by Characterizing the Transcriptomic Features in Clinical Tumor Samples across 15 Cancer Types,” Online, Computational Intelligence and Neuroscience 2022: 5443709; Zhang, Z., Dong. Y., Wu. B., Li, Y., Liu, Z., and 6 others 2022. “Irradiation Enhances the Malignancy-promoting Behaviors of Cancer-associated Fibroblasts,” Online, Frontiers in Oncology 12:965660; Zhou, J., Lei, N., Tian, W., Guo. R., Chen, M., and 4 others 2022. “Recent Progress of the Tumor Microenvironmental Metabolism in Cervical Cancer Radioresistance,” Online, Frontiers in Oncology 12:999643; Zhou, R., Zhao, D., Beeraka, N. M., Wang, X., Lu, P., and 3 others 2022. “Novel Implications of Nanoparticle-enhanced Radiotherapy and Brachytherapy Z-effect [effective atomic number/effective nuclear charge] and Tumor Hypoxia,” Online, Metabolites 12(10): 943; Kabakov, A, E. and Yakimova, A. O. 2021. “Hypoxia-induced Cancer Cell Responses Driving Radioresistance of Hypoxic Tumors: Approaches to Targeting and Radiosensitizing,” Online, Cancers 13(5): 1102; Begg, K. and Tavassoli, M. 2020. “Inside the Hypoxic Tumour: Reprogramming of the DDR [DNA damage response] and Radioresistance,” Cell Death Discovery 6:77; Olivares-Urbano, M. A., Grinan-Lison, C. Marchal, J. A., and Nunez, M. I. 2020. “CSC [cancer stem cells] Radioresistance: A Therapeutic Challenge to Improve Radiotherapy Effectiveness in Cancer Cells,” Online, Cells 9(7): 1651; Bhandari, V., Hoey, C., Liu, L. Y., Lalonde, E. Ray, J., and 18 others 2019. “Molecular Landmarks of Tumor Hypoxia Across Cancer Types,” Nature Genetics wdqw51:308; Citrin, D, E. 2017. “Recent Developments in Radiotherapy,” New England Journal of Medicine 377(11): 1065-1075; Lee, S. Y., Jeong, E. K., Ju, M. K., Jeon, H. M., Kim, M. Y., and 4 others 2017. “Induction of Metastasis, Cancer Stem Cell Phenotype, and Oncogenic Metabolism in Cancer Cells by Ionizing Radiation,” Online, BioMed Central Molecular Cancer 16(1): 10; Chang, L., Graham, P., Hao. J., Ni. J., Deng. J., and 4 others 2016. “Cancer Stem Cells and Signaling Pathways in Radioresistance,” Oncotarget 7(10): 11002-11017; Chao, L., Lin, Q., and Yuna, Z. 2015. “Cellular and Molecular Mechanisms Underlying Oxygen-dependent Radiosensitivity,” Radiation Research 183(5): 487-496; Gee, H. E., Ivan, C., Calin, G. A., and Ivan, M. 2014. “HypoxamiRs [overexpression of the mir-210 gene] and Cancer: From Biology to Targeted Therapy,” Online, Antioxidants and Redox Signaling (Larchmont, New York) 21:1220-1238; Rockwell, S., Dobrucki, I. T. Kim, E. Y., Marrison, S. T., and Thus Vu. V. 2009. “Hypoxia and Radiation Therapy: Past History, Ongoing Research, and Future Promise,” Current Molecular Medicine 9(4): 442-458).

Means for minimizing injury to healthy oxygenated tissue surrounding a tumor include reducing the oxygen level of the surrounding tissue through short term or intermittent constriction of its arterial supply. The additional release of sulfhydryl and other compounds, known for over a half century to induce hypoxia, through an accessory channel of the servochoke used to effectuate the constriction can be used to increase the hypoxia (references below in this section). Broadly, various substances known to reduce or increase the vulnerability of DNA to radiation can be delivered to the treatment site through one or more accessory channels in a side-entry valve, servochoke, or connector.

FIGS. 14B and 14C schematically depict the treatment of a malignant tumor within a kidney using this approach. If successful in destroying the lesion, such treatment eliminates the need for resection with considerable trauma to the kidney. Shown in FIG. 26B, the components comprising the drug delivery path-subdermally implanted body surface port backplate 100 shown in greater detail in FIG. 26A, delivery lines 8 (effectively the upstream origins of the accessory channels, or sidelines to the nonjacketing side-entry connector 165, from the port to drug reservoirs 102, reservoir outlet drip device or depending upon the viscosity of the agent, pump 103, drugline 8 from the outlet pump or drip device 103 which can be the mainline or sideline (accessory channel), and nonjacketing side-entry connector 165- and servochoke 166 are radiation shielded only when the polymer or coated polymer of which these are made fails to provide adequate shielding.

In FIGS. 14B, 14C, and 26B, inline axial or peristaltic, reversible pump 103 outlets through drugline 8 made of a durable tubing such as Phar Med BPT (Compagnie de Saint Gobain S. A. (LaDefense and Courbevoie, France) or a double layer drugline with internal polytetrafluoroethylene and outer pliant polymer layer such as plasticized polypropylene or polyetheretherketone, for example. A servochoke is shown in FIGS. 14A and 14B. The type nonjacketing side-entry connector 165—is described and illustrated in U.S. Pat. No. 11,013,858, entitled Nonjacketing Side-entry Connectors and Prosthetic Disorder Response Systems, in FIGS. 13A thru 15. As in most cases, unless causing the patient discomfort, the components in the arrangement are not removed following treatment but left in place should the need for further treatment to benefit from the direct drug targeting provided by the druglines arise.

FIG. 14B depicts a ‘two-pronged attack’ on a malignant tumor in which the blood supply to the affected organ-here the renal artery—is constricted by servochoke 166 to induce hypoxia in the healthy tissue surrounding the tumor (as well as in the tumor). At the same time, a radiation shielded nonjacketing side-entry connector positioned along the outer surface of the kidney secures the open distal end of its shielded mainline inside the tumor to release a radioactive drip. Because it enhances hypoxia in the tumor, the antiangiogenic approach of Folkman is incompatible with and should not be used concurrently with radiotherapy. A safe and effective method for irradiating a malignant tumor 1. Minimizes if not eliminates spillover to healthy tissue surrounding the tumor, and 2. Does not allow the venous outflow to include a radioactive residue as would expose the endothelium to radiation.

Temporary radiation shielding—that suitable for this application—and permanent radiation shielding such as depicted in FIG. 14C is described and illustrated in U.S. Pat. No. 11,013,858 just mentioned, and below in section 6 entitled Description of the Preferred Embodiments of the Invention. As indicated, the incidence of radiation spillover—the central deterrent to the use of conventional radiotherapy, which is prone to destroy the oxygenated healthy tissue surrounding a malignant tumor even more than the hypoxic tissue of the tumor itself—is virtually eliminated by directly pipeline-targeting a radioactive drip with or without additives such as a sulfhydryl or similar compound into the tumor. FIG. 14A shows a servochoke that appear as part number 166 in FIGS. 14B and 14C to adjust the local blood pressure.

One or more accessory channels of the nonjacketing side-entry connector can be used to deliver a nanoparticulate containing fluid that specifically oxygenates the tumor. Several types of such agents have been developed, of which several are cited below. These are deliverable to the target tumor through one or more accessory channels of the radiation shielded or nonshielded nonjacketing side-entry connector securing the radioisotope delivery mainline in place at the surface of the affected organ.

Whether the latter would best be mixed in with the radioisotope for delivery through the same line depends upon the chemistry of both. Separate accessory channels used to deliver oxygenating substances are shielded or not as necessary. Such a brachytherapeutic drip is included in a totally implanted automatic disorder response system for control thereby only when appropriate or an eventual need therefor is anticipated. Once the tumor has been eradicated and the drip stopped, the connector and its mainline can be left in place indefinitely, eventually to be redirected to another use.

While directly pipeline-targeted drip brachytherapy or brachyradiochemotherapy is discussed in the context of incorporation into a totally implanted automatic disorder response system, in principle, it can be readily adapted for manual use endoscopically to provide greater focus on the target than conventional brachytherapy or teletherapy. As with fixation in position at the outer fibrous layer with a nonjacketing side-entry connector, manual application involves inserting the distal tip of the drip probe into the tumor through a stab wound.

Although not usable automatically by the implant disorder control system as is a pipeline-targeted radioactive drip, provided with an epicutaneous (body surface, on-skin, extracutaneous) port such as shown in FIG. 26C and in FIGS. 28, and 30 as part number 109 and a guide tube from the port to the target, a laser with steerable leading end can be used without the need for reentry or relocating the treatment site. U.S. Pat. No. 11,013,858, entitled Nonjacketing Side-entry Connectors and Prosthetic Disorder Response Systems, FIGS. 13-16 shows a mechanism for advancing the probe into the tumor over a controlled interval. Epicutaneous ports are generally limited to those incorporating an outlet for an excurrent or outflow line, such as in a urinary prosthesis. Otherwise, injection points, for example, even when belonging to the same port are subcutaneous with the needle insertion point or points indicated by a tattooed dot or dots.

In contrast, used endoscopically without a guide catheter, the elimination of invasive reentry and the need to relocate the treatment site during each visit that is an inherent advantage of implantation is lost, and surgical rather than guide tube reentry required, the manual use of a conventional laser may be equally effective. With the automatic probe advancement device implanted, the need for reentry occurs only once to remove the device after the tumor has been confirmed destroyed. And because each use thereof demands revisiting the clinic, a laser used manually or guided is less expedient than is a drip, of which the use is fixedly positioned eliminating not just the need for reentry but imaging to relocate the treatment site.

Thus, given that fixed in position and integrated into the prescription-program, the drip responds immediately under automatic control with the patient ambulatory and oblivious to its operation, the drip is more functional than either a prepositioned guide tube or a manual laser. To replenish the drug reservoir, a system incorporated drip requires a subdermal port such as shown in FIG. 26A. Again, when the agent is radioactive, the port, reservoir, and all other parts of the track as shown in FIG. 14C are shielded.

The process is amenable to the incorporation through adaptation of allied technology (see for example, Wang, W., Wang, X., Tao, F., Hu, K., Zhang, J., and 3 others 2022. “Fluorinated Hyaluronic Acid Encapsulated Perfluorocarbon Nanoparticles as Tumor-targeted Oxygen Carriers to Enhance Radiotherapy,” American Chemical Society Molecular Pharmaceutics. 19(11): 3948-3958; Zhou X., You M., Wang F., Wang Z., Gao X., and 8 others 2021. “Multifunctional Graphdiyne-Cerium Oxide Nanozymes Facilitate MicroRNA Delivery and Attenuate Tumor Hypoxia for Highly Efficient Radiotherapy of Esophageal Cancer,” Advanced Materials (Deerfield Beach, Florida) 33(24): e2100556; Gao, M., Liang, C., Song, X., Chen, Q., Jin, Q., Wang, C., and Liu, Z. 2017. “Erythrocyte-membrane-enveloped Perfluorocarbon as Nanoscale Artificial Red Blood Cells to Relieve Tumor Hypoxia and Enhance Cancer Radiotherapy,” Advanced Materials (Deerfield Beach, Florida) 29(35): 1701429).

Once the radioactive drip has materially reduced the size of the tumor so that unaffected tissue surrounding it is brought within range, the hypoxic state of the tumor compared to the unaffected tissue that rendered the tumor less susceptible to ionizing radiation biases cell destruction in favor of the healthy over the tumor cells. Thus, temporarily inducing an hypoxic state in the healthy tissue removes this bias, and inducing a more pronounced hypoxia in the healthy tissue reverses the bias to allow a clearer delineation of the border at which the destruction of cells is to stop.

Except in the elderly, in whom the use thereof would alleviate pain but who would die before late-term sequelae of irradiation would have time to develop, the system is configured to prevent the irradiation of epithelia—in FIG. 14A, the supply artery bypassed, specifically that of the vein-especially the exquisitely radiosensitive endothelium lining vessels and the urothelium lining the urinary tract. In most cases, the use of directly pipeline-targeted drip brachytherapy should result in no radiation exposure within the supply artery and at most negligible exposure within the drainage vein.

The scheme for using directly pipeline-targeted drip brachytherapy as shown in FIG. 14A thus substantially avoids the need for vascular shielding. Accordingly, especially in an elderly or terminal patient, to assure complete protection of the large vessels by ensheathment within shielding as would extend the procedural time, the use of extended shielding is best discounted. In such patients, where the vein is intimately associated with the carcinoma or carcinosarcoma to be eliminated in such a way that allowing a radioactive residue to pass through the vessels should yield more benefit than risk, extended shielding should be avoided.

The tumor already hypoxic, constriction of the supply artery differentially affects the normally oxygenated tissue surrounding the tumor more than the tissue of the tumor. The hypoxic state is allowed only when the radioisotope is released into the tumor and only once the border comes into reach of the emission track range. Substances known to reduce the vulnerability of DNA to radiation over and above an hypoxic state are delivered through one or more accessory channels of the servochoke shown in detail in FIG. 14A used to constrict the blood supply, as depicted in FIG. 14B, specifically the renal artery. Continuously variable adjustment in the rate of flow-through volume could also be accomplished with a tuned shunt. FIG. 14C shows the same device with radiation shielding.

Methods for minimizing the effect of spillover consist of reducing the oxygen level of the healthy tissue surrounding the tumor such as with the use of sulfhydryl compounds, and for expediting the eradication of the tumor, increasing its oxygen level with nanoparticulate preparations the details appurtenant thereof addressed below. Accessory channels allow the application of both methods simultaneously. Constriction of the supply artery during radiotherapy is possible apart from incorporation into an automatic response system; however, in such a system, the use thereof is automatically optimized in that the sequence of actions is continuously monitored and coordinated in response to feedback supplied by implanted sensors.

To render the healthy tissue in the organ less susceptible to spillover, its level of oxygenation is reduced, and to increase the level of oxygen in the tumor thus increasing its vulnerability to the radiation delivered by the radioactive drip, one or more servochoke accessory channels can be used to concurrently deliver new materials that promote oxygenation in the malignant tissue.

The induction of hypoxia in healthy tissue in order to protect it from radiation directed at a contiguous tumor is addressed above in this section which provides additional references to the literature (see, for example, Hajizadeh, F., Ardebili, S. M., Moomani, B., Masjedi, A., Atyabi, F. and 10 others 2020, Op cit.; Kim, S. W., Kim, I. K., and Lee, S, H. 2020. “Role of Hyperoxic Treatment in Cancer,” Experimental Biology and Medicine (Maywood, New Jersey) 245(10): 851-860; Mi. P. 2020. “Stimuli-responsive Nanocarriers for Drug Delivery, Tumor Imaging. Therapy, and Theranostics,” Theranosties (Wyoming New South Wales, Australia) (10(10): 4557-4588; Sun, H. Wang. X., and Zhai S. 2020. “The Rational Design and Biological Mechanisms of Nanoradiosensitizers,” Nanomaterials (Basel, Switzerland) 10(3): 504; Wang, J., Sun, J., Hu, W., Wang. Y., Chou, T., and 4 others 2020. “A Porous Au@Rh Bimetallic Core-Shell Nanostructure as an H2 O₂-driven Oxygenerator to Alleviate Tumor Hypoxia for Simultaneous Bimodal Imaging and Enhanced Photodynamic Therapy,” Advanced Materials (Deerfield Beach, Florida) 32(22): e2001862; Du. B., Ding, X., Wang. H., Du, Q., Xu. T., and 3 others 2019. “Development of an Interactive Tumor Vascular Suppression Strategy to Inhibit Multidrug Resistance and Metastasis with pH/H₂O₂ Responsive and Oxygen-producing Nanohybrids,” Journal of Materials Chemistry. Part B 7(31): 4784-4793; Fu, C., Duan, X., Cao. M., Jiang, S., Ban, X., and 6 others 2019. “Targeted Magnetic Resonance Imaging and Modulation of Hypoxia with Multifunctional Hyaluronic Acid-MnO2 Nanoparticles in Glioma,” Advanced Healthcare Materials 8(10): e1900047; Jia. T.-T., Yang, G., Mo. S.-J., Wang, Z.-Y., Li B.-J., and 6 others 2019. “Atomically Precise Gold-levonorgestrel Nanocluster as a Radiosensitizer for Enhanced Cancer Therapy,” American Chemical Society Nano,” 13(7): 8320-8328; Wang, Y., Shang. W., Niu, M., Tian, J., and Xu, K. 2019. “Hypoxia-active Nanoparticles Used in Tumor Theranostic,” International Journal of Nanomedicine 14:3705-3722; Xie, J., Gong, L., Zhu, S., Yong, Y., Gu, Z., and Zhao, Y. 2019. “Emerging Strategies of Nanomaterial-mediated Tumor Radiosensitization,” Advanced Materials (Deeffield Beach, Florida) 31(3): 1802244; Zhang, C., Yan, L., Gu, Z., and Zhao, Y. 2019. “Strategies Based on Metal-based Nanoparticles for Hypoxic-Tumor Radiotherapy,” Chemical. Science 10(29): 6932-6943; Zhong, D., Zhao, J., Li, Y., Qiao, Y., Wei, Q., and 4 others 2019. “Laser-triggered Aggregated Cubic α-Fe2O3@ Au Nanocomposites for Magnetic Resonance Imaging and Photothermal/Enhanced Radiation Synergistic Therapy,” Biomaterials 219:119369; Zhou, J., Xue. C., Hou Y. Li, M., Hu, Y., and 5 others 2019. “Oxygenated Theranostic Nanoplatforms with Intracellular Agglomeration Behavior for Improving the Treatment Efficacy of Hypoxic Tumors,” Biomaterials 197:129-145; Cheng, K., Sano, M., Jenkins, C. H., Zhang, G., Vernekohl, D., and 7 others 2018. “SynergisticallyEnhancing the Therapeutic Effect of Radiation Therapy with Radiation Activatable and Reactive Oxygen species-releasing Nanostructures,” American Chemical Society Nano 12(5): 4946-4958; Choi, B. J., Jung, K. O., Graves, E. E., and Pratx, G. 2018. “A Gold Nanoparticle System for the Enhancement of Radiotherapy and Simultaneous Monitoring of Reactive-oxygen-species Formation,” Nanotechnology 29(50): 504001; Graham, K. and Unger, E. 2018. ““Overcoming Tumor Hypoxia as a Barrier to Radiotherapy, Chemotherapy and Immunotherapy in Cancer Treatment,” International Journal of Nanomedicine 13:6049-6058; Jahanban-Esfahlan, R., de la Guardia, M., Ahmadi, D., and Yousefi, B. 2018. “Modulating Tumor Hypoxia by Nanomedicine for Effective Cancer Therapy,” Journal of Cellular Physiology 233(3): 2019-2031; Klein, S., Hubner, J., Menter, C., Distel, L. V. R., Neuhuber, W. and Kryschi, C. 2019. “A Facile One-pot Synthesis of Water-soluble, Patchy Fe₃O₄—Au Nanoparticles for Application in Radiation Therapy,” Applied Sciences 9(1): 15; Li, J., Shang. W., Li, Y., Fu, S., Tian, J., and Lu, L. 2018. “Advanced Nanomaterials Targeting Hypoxia to Enhance Radiotherapy,” International Journal of Nanomedicine 13:5925-5936; Li, N., Yu, L., Wang, J., Gao, X., Chen, Y., Pan, W., and Tang, B. 2018. “A Mitochondria-targeted Nanoradiosensitizer Activating Reactive Oxygen Species Burst for Enhanced Radiation Therapy,” Chemical. Science 9(12): 3159-3164; Liu, Y., Zhang, P., Li, F., Jin, X., Li, J., Chen, W., and Li, Q. 2018. “Metal-based Nanoenhancers for Future Radiotherapy: Radiosensitizing and Synergistic Effects on Tumor Cells,” Theranostics (Wyoming New South Wales, Australia) 8(7): 1824-1849; Penninckx, S., Heuskin, A.-C., Michiels. C., and Lucas, S. 2018 “The Role of Thioredoxin Reductase in Gold Nanoparticle Radiosensitization Effects,” Nanomedicine (London, England) 13(22): 2917-2937; Liu, J.-N., Bu, W., and Shi, J. 2017. “Chemical Design and Synthesis of Functionalized Probes for Imaging and Treating Tumor Hypoxia,” Chemical Reviews 117(9): 6160-6224; Liu, Y., Zhang, P., Li, F., Jin, X., Li, J., Chen, W., and Li, Q. 2018. “Metal-based Nanoenhancers for Future Radiotherapy: Radiosensitizing and Synergistic Effects on Tumor Cells,” Theranostics (Wyoming New South Wales, Australia) 8(7): 1824-1849; Fang, X., Wang, Y., Ma, X., Li, Y., Zhang, Z., and 4 others 2017. “Mitochondria- targeting Au Nanoclusters Enhance Radiosensitivity of Cancer Cells,” Royal Society of Chemistry Journal of Materials Chemistry. Part B. [Biology and Medicine] 5(22): 4190-4197; Ma, N., Liu, P., He, N., Gu, N., Wu, F.-G., and Chen, Z. 2017. “Action of Gold Nanospikes-based Nanoradiosensitizers: Cellular Internalization, Radiotherapy, and Autophagy.” ACS [American Chemical Society] Applied Materials and Interfaces 9(37): 31526-31542; Rey, S., Schito, L., Koritzinsky, M., and Wouters, B, G. 2017. “Molecular Targeting of Hypoxia in Radiotherapy,” Advanced Drug Delivery Reviews 109:45-62; Yang, S. and Gao, H. 2017. “Nanoparticles for Modulating Tumor Microenvironment to Improve Drug Delivery and Tumor Therapy,” Pharmacological Research 126:97-108; Song. M., Liu, T., Shi, C., Zhang, X., and Chen, X. 2016. “Bioconjugated Manganese Dioxide Nanoparticles Enhance Chemotherapy Response by Priming Tumor-associated Macrophages toward M1 [inflammation encouraging macrophage]-like Phenotype and Attenuating Tumor Hypoxia,” American Chemical Society Nano 10(1): 633-647; Thambi, T., Park, J. H., and Lee, D. S. 2016. “Hypoxia-responsive Nanocarriers for Cancer Imaging and Therapy: Recent Approaches and Future Perspectives,” Chemical Communications (Cambrige, England) 52(55): 8492-8500; Tian, J., Chen, J., Ge, C., Liu, X., He, J., Ni, P., and Pan, Y. 2016. “Synthesis of PEGylated Ferrocene Nanoconjugates as the Radiosensitizer of Cancer VCells,” Bioconjugate Chemistry 27(6): 1518-1524; Cooper, D. R., Bekah, D., and Nadeau, J. L. 2014. “Gold Nanoparticles and Their Alternatives for Radiation Therapy Enhancement,” Online, Frontiers in Chemistry 2:86; Prasad, P., Gordijo, C. R., Abbasi, A. Z., Maeda, A. Ip, A. and 3 others 2014. “Multifunctional Albumin-MnO₂ Nanoparticles Modulate Solid Tumor Microenvironment by Attenuating Hypoxia, Acidosis, Vascular Endothelial Growth Factor and Enhance Radiation Response,” American Chemical Society Nano 8(4): 3202-3212; Zheng, Q., Yang, H., Wei, J., Tong, J.-I., and Shu, Y.-q. 2013. “The Role and Mechanisms of Nanoparticles to Enhance Radiosensitivity in Hepatocellular Cell,” Biomedicine and Pharmacotherapy 67(7): 569-575; Geng, F., Song, K., Xing, J. Z., Yuan, C., Yan, S., and 3 others 2011. “Thioglucose-bound Gold Nanoparticles Enhance Radio-cytotoxic Targeting of Ovarian Cancer,” Nanotechnology 22(28): 285101).

Chemotherapy may complement radiation in the destruction of malignant, but normal cells as well. As can all other components in the system, servochokes can be radiation shielded; however, the supply artery the servochoke surrounds should not see ionizing radiation. Schematically represented to the left in FIG. 14C, the shielded radioisotope drug reservoir outlets to a bidirectional rotary solenoid-controlled drip stopcock or roller clamp followed by a drip counting chamber, the rate of the drip regulated by the implanted microcontroller or microprocessor which coordinates the timing sequence of actions among the components.

Release of the radioisotope into the tumor is preceded by actuation of the servochoke which constricts the renal artery to induce an hypoxic state in the unaffected tissue surrounding the tumor, thus dispelling the bias for damaging normally oxygenated more than the hypoxic tissue of the tumor. Control over the drip rate can be accomplished by different mechanical devices, some requiring a microminiature linear servomotor. While gravity-dependent and not actually pumps, an infusion ‘pump’ can substitute for an actual pump such as peristaltic for liquid drugs of moderate viscosity.

Additionally or used alternatively, the inducement of hypoxia in the tissue surrounding the tumor renders it less radiosensitive, or more radioresistant, which effect can be augmented by delivery through a nonjacketing side-entry connector accessory channel of a sulfhydryl compound, for example (Fujimoto, T. N., Colbert, L. E., Huang, Y., Molkentine, J. M., Deorukhkar, A., and 13 others 2019. “Selective EGLN [hypoxia-inducing enzyme encoding gene] Inhibition Enables Ablative Radiotherapy and Improves Survival in Unresectable Pancreatic Cancer,” Cancer Research 79(9): 2327-2338; Hahn, S. M. and Glatstein, E. 2005. “Principles of Radiation Therapy,” in Kasper, D. L. Braunwald, E., Fauci, A. S., Hauser, S, L., Longo, D, L., and Jameson, J. L. Harrison's Principles of Internal Medicine, Chapter 71, page 467, New York, New York: McGraw-Hill Medical Publishing Division), Chapter 70, page 485; Hospers, G, A. P., Eisenhauer, E. A., and de Vries, E. G. E. 1999. “The Sulfhydryl Containing Compounds WR-2721 and Glutathione as Radio- and Chemoprotective Agents. A Review, Indications for Use and Prospects,” British Journal of Cancer 1999 80(5-6): 629-638; Radi. R., Beckman, J. S., Bush, K. M., and Freeman, B, A. 1991. “Peroxynitrite Oxidation of Sulfhydryls: The Cytotoxic Potential of Superoxide and Nitric Oxide,” Journal of Biological Chemistry 266:4244-4250). This approach does not apply to the use of cisplatin or 5-fluorouracil which increase the radiation susceptibility of cells in a hypoxic tumor (Chen, Y-J., Tsai, T.-H. Wang, L-Y., and Hsieh, C, H. 2017. “Local Radiotherapy Affects Drug Pharmacokinetics-Exploration of a Neglected but Significant Uncertainty of Cancer Therapy,” Technology in Cancer Research and Treatment 16(6): 705-716). Fochem, K., Michalica, W., and Picha, E. 1967. “On the Effect of Sulfhydryl Compounds as Radiation Protective Agents in combination with fructose,” (in German with English abstract at Pubmed) Strahlentherapie [Radiotherapy] 134(2): 279-283; In some instances, radiation injury to nearby healthy cells can be counteracted through the concurrent pipeline-targeted delivery of protective substances sulfhydryl and others through accessory channels (see, for example, Cohen, E. P. Fish, B, L., and Moulder, J, E. 2010. “Mitigation of Radiation Injuries via Suppression of the Renin-angiotensin System: Emphasis on Radiation Nephropathy,” Current Drug Targets 11:1423-1429; Robbins, M. E., Zhao, W., Garcia-Espinosa, M. A., and Diz, D. I. 2010. “Renin-angiotensin System Blockers and Modulation of Radiation-induced Brain Injury,” Current Drug Targets 11:1413-1422).

The use of radiation shielding pertains equally to the protection of both nonradioactive drugs moving through mainlines or accessory channels from surrounding radiation and to tissue neighboring lines passing radioactive substances. While ionizing radioactive agents moving through accessory channels will be in the form of liquids, any radiation outside such lines will usually have been produced by external beam or brachytherapeutic sources. In elderly patients with multiple comorbidities taking multiple drugs, both type sources may be required in close relation in different parts of the body, potentially resulting in radiation-drug interactions that alter drug structure and metabolism (pharmacokinetics and/or pharmacodynamics).

Such consequences are more complicated in the elderly but apply wherever drugs and radiation can interact (see, for example, Chen, Y-J., Tsai. T.-H. Wang, L-Y., and Hsieh, C, H. 2017, Op cit.; Silindir, M. and Ozer, Y. 2012. “The Effect of Radiation on a Variety of Pharmaceuticals and Materials Containing Polymers,” Parenteral Drug Association Journal of Pharmaceutical Science and Technology 66(2): 184-199) as well as their metabolism (see, for example, Li, X., Yang, J., Qiao, Y., Duan, Y., Xin, Y., Nian, Y., Zhu, L., and Liu, G. 2019. “Effects of Radiation on Drug Metabolism: A Review,” Current Drug Metabolism 20(5): 350-360). The implanted microcontroller or microprocessor can alternate the delivery through the same mainline or the same or a different accessory channel of different radiation regimens to kill the cells or destroy the vasculature of a malignant tumor (Chen, Y-J., Tsai. T.-H. Wang, L-Y., and Hsieh, C, H. 2017, Op cit.).

Apart from applications of an implanted automatic disorder response system in surgical practice to directly pipeline-target drugs to specific organs or tissues, to achieve a normal ventricular absolute stroke volume, or ejection fraction, from, for example, a solitary or double sudden or metered switch transplanted heart as addressed below, intravascular diversion servovalves are accompanied by and controlled to cooperate with choke servovalves, or servochokes, or sphincteric servovalves, addressed below in section 2b(2)(i), entitled Vascular Servochokes, and the subsections thereto. Compound servovalves incorporate both diversion and choke capabilities.

Any basic side-entry, diversion, and/or chokevalve can be radiation shielded to convey radionuclides, for example, higher dose rate or intensity than the polymeric material of which the component is made and can incorporate a circumferential layer magnetized radially in relation to the long axis of the substrate ductus and encircling jacket to draw nanoparticulate superparamagnetic carrier-bound drugs, for example. Radiation shielding is described and illustrated in U.S. Pat. No. 11,013,858, entitled Nonjacketing Side-entry Connectors and Prosthetic Disorder Response Systems, and U.S. Pat. No. 11,759,186, entitled Ductus Side-entry Jackets and Prosthetic Disorder Response Systems, but in elderly patients, can apply as much to basic ductus side-entry jackets and vascular valves and servovalves as much as to nonjacketing side-entry connectors, any or all of which may be used in an implanted prosthetic disorder response system.

The passage of radioactive agents through native ductus, as opposed to synthetic accessory channels, limited to cases where the vessel or urethra is intimately associated with the malignant tissue, radiation shielding in side-entry jacket and valves pertains to need to protect agents delivered into these through their accessory channels. Seen thus, shielding is more often needed to protect against sources of radiation external to jackets and connectors rather than neighboring tissue from radioactive substances flowing thorough the accessory channels of side-entry jackets, valves, and connectors.

And if the pulmonary vascular resistance is more pronounced, the metered compound vascular bypass, or sudden switched-organ transplantation technique, administered by an implanted control system, applicable to any organ or combination of organs, can not only optimize the transplantation but provide immediate medicinal, and/or electrostimulatory support to improve the prospects for a heterotopic heart or a heart-lung transplant (see, for example, Reitz, B. A., Wallwork, J, L., Hunt, S. A., Pennock, J, L., Billingham, M. E., and 3 others 1982. “Heart-Lung Transplantation: Successful Therapy for Patients with Pulmonary Vascular Disease,” New England Journal of Medicine 306(10): 557-564; additional references to lung transplantation are provided below in section 2b(1)(a) entitled Sudden Compound Vascular Bypass, or Sudden Switched-organ Transplantation).

While the term ‘heterotopic’ does apply to experimental positioning of a second heart in the upper abdomen, infrarenally in the lower abdomen, or in the pelvis of laboratory test animals, it is not properly applied to the ‘piggyback’ procedure, which infrequently performed, represents conventional surgery in humans to position donor and recipient merged hearts orthotopically. Conventionally denoting the nonexperimental conventional merged, or ‘piggyback’ configuration, even though positioning of the donor heart in double heart transplantation as described herein may indeed be heterotopic, use of the term ‘heterotopic’ in conjunction with a double heart transplantation as addressed below in section 2b(1)(b)(b5), entitled Heterotopic Double Heart Metered Compound Vascular Bypass, or Sudden Switched-organ Transplantation [and so on], is avoided.

Metered compound vascular bypass, or sudden switched-organ transplantation directly addresses the causes for rejection and physiological inadequacy, meaning the immune response to allogeneic tissue and the ischemia-reperfusion injury respectively to which the tissue had been subjected in a corresponding conventional procedure. Procedures described herein are preferably undertaken using regional rather than general anesthesia. While the procedures, such as a heart transplant or replacement of a segment along the aorta should not inherently generate more than moderate pain, the unavoidably extensive and deep entry wound essential to gain access pertains equally to any such procedure regardless of the method used. Unsuppressed, the intense pain associated with a clamshell or hemiclamshell incision, for example, would overwhelm any relatively minor diminution in pain associated with an intracorporeal procedure itself inherently less painful.

The degree of reperfusion injury, injury to the electrical conduction system of the heart (cardionector, cardionecteur, natural cardiac pacemaker), and synchronization problems following transplantation is attributable and directly proportional to the ischemia time preceding insertion into the circulatory system of the recipient (see, for example, Wdowczyk, J., Makowiec, D., Gruchala, M., Wejer, D., and Struzik, Z, R. 2018, Op cit., this section, Kalogeris. T., Baines, C. P., Krenz, M., and Korthuis, R. J. 2016. “Ischemia/Reperfusion,” Comprehensive Physiology 7(1): 113-170; Kalogeris, T., Baines, C. P., Krenz, M., and Korthuis, R. J. 2012. “Cell Biology of Ischemia/Reperfusion Injury,” International Review of Cell and Molecular Biology 298:229-317; Thajudeen. A., Stecker, E. C., Shehata, M., Patel, J., Wang, X., and 3 others 2012. “Arrhythmias after Heart Transplantation: Mechanisms and Management,” Journal of the American Heart Association (Oxford, England) 1(2): e001461; additional references below in this section).

A central benefit in compound vascular bypass, or sudden switched-organ transplantation is the complete elimination of ischemia, meaning the graft organ is never subjected to an arrest in perfusion and anoxia. Compound vascular bypass solid organ transplantation seeks to minimize the loss of perfusion, the trauma of reperfusion, and the shock of resituation in an immunologically alien context as sources of degradation in a graft organ which collaborate to reduce its survivability and likelihood of failure. The reactions to all three are functions of the immune system, present even were the organ an autograft after having undergone ischemia, and fundamentally more intense when provoked by an allograft. In switch transplantation, ischemia time, hence, reperfusion injury, is zero, and especially when switch transplantation is metered, graft organ injury in the form of reperfusion injury, vasculopathy, and nervous impairment, all interrelated, should be minimized if not eliminated due to the immune tolerance induction integral in the process of graft organ transfer from the circulatory system of the donor into that of the recipient.

It thus deals with both sets of insults which collaborate to cause graft failure. “After heart transplantation, the allograft undergoes characteristic alterations in myocardial structure, including hypertrophy, increased ventricular stiffness, ischemia, and inflammation which, together with the natural process of aging, may lead to vasculopathy and fibrosis of the donor heart” (Wdowczyk, J., Makowiec, D., Gruchala, M., Wejer, D., and Struzik, Z, R. 2018, Op cit., second paragraph above). (In the references cited, an allograft may be referred to as an allogeneic graft, homogeneic graft, homograft, homeograft, homeotransplant, homogenous graft, homologous graft, homoplastic graft, or homotransplant.)

A frequent problem encountered in heart transplantation is the need to safely remove transvenous pacemaker, cardioverter defibrillator or coronary sinus leads which had been needed with the native heart of the recipient. This topic, pertinent to orthotopic heart transplantation regardless of the technique employed, and to heterotopic switch double heart metered compound vascular bypass, or sudden switched-organ transplantation where due to the unavailability of a sound replacement for the native heart, clinical judgement should support replacement with a second heart, is addressed below in section 2b(1) entitled Intercorporeal Diversion of Blood Flow.

Aside from the advantages over both conventional orthotopic and heterotopic methods, that some 25 percent of patients on a waiting list for a replacement heart die and that the waiting time in many parts of the world has been growing over the past two decades warrants the increased availability of hearts as well as other organs which the switch method of orthotopic and metered double heart transplants would allow (see, for example, Morris, A. A., Chen, C., Laskar, S. R., Smith, A, L., and Vega, J, D. 2019. “Mortality in Status 2 Patients Listed for Heart Transplantation in the United States: Will Understanding Cause of Death Help Justify Implantation of Left Ventricular Assist Devices into Less Sick Patients?,” International Journal of Cardiology 276:185-190; Blackstone, E. H., Rajeswaran, J., Cruz, V. B., Hsich, E. M., Koprivanac, M., and 4 others 2018. “Continuously Updated Estimation of Heart Transplant Waitlist Mortality,” Journal of the American College of Cardiology 72(6): 650-659; Jasseron, C., Legeai, C., Jacquelinet, C., Leprince P., Cantrelle, C., and 4 others 2017. “Prediction of Waitlist Mortality in Adult Heart Transplant Candidates: The Candidate Risk Score,” Transplantation 101(9): 2175-2182; Goldstein, B. A., Thomas, L., Zaroff, J. G., Nguyen, J., Menza, R., and Khush, K. K. 2016. “Assessment of Heart Transplant Waitlist Time and Pre- and Post-transplant Failure: A Mixed Methods Approach,” Epidemiology (Cambridge, Massachusetts) 27(4): 469-476; Jeewa, A., Manlhiot, C., Kantor, P. F., Mital, S., McCrindle, B. W., and Dipchand, A. I. 2014. “Risk Factors for Mortality or Delisting of Patients from the Pediatric Heart Transplant Waiting List,” Journal of Thoracic and Cardiovascular Surgery 147(1): 462-468).

Currently, end-stage or terminal patients who agree to donate their organs are allowed to undergo cardiac death without having been maintained on life support. Unless the primary cause of death is cardiogenic, such as in a sudden arrest or sudden cardiac death, then severed from the higher centers that govern the action of the heart, the intrinsic timing circuit, or conduction system (cardionector, cardionecteur) of the heart, continues the beat but without stability, even before the graft organ, whether the heart, liver, kidneys, lungs, pancreas, gut, or a gland is harvested. This, even though the continuation of normal perfusion by normothermic autologous blood past cardiac death would spare degradation in the organs, glands, and tissues.

Thus, hypoxia commences prior to transplantation only to be joined by reperfusion injury and the immune reaction when the organ is transplanted. Of the two principal causes for transplant organ failure, ischemia-reperfusion injury can be avoided entirely, and rejection more effectively suppressed by the targeted release of immunosuppressants at the supply of the graft organ or organs. An implanted disorder response system can directly switch the circulation of the recipient to and through the donor organ by means of vascular valves, and moreover, directly pipeline-target drugs such as immunosuppressants to the treatment site.

By continuously monitoring and directly pipeline-targeting drugs to treat any of a number of comorbid conditions, the use of an implanted automatic disorder response system as described in U.S. Pat. No. 11,759,186, entitled Ductus Side-entry Jackets and Prosthetic Disorder Response Systems should liberalize the zone of patients able to undergo a heart transplant, left ventricular aneurysmal reconstructive surgery, or enlarged volume reduction surgery, whether using the Batista reduction left ventriculoplasty procedure for partial left ventriculectomy, or Dor endoventricular circular patch plasty suturing procedure which has been found to provide more dependable outcomes.

That drugs such as immunosuppressants, angiotensin converting enzyme inhibitors, anti-inflammatoires, and antimicrobials can be squarely targeted to the graft or surgically treated organ avoids exposure of other tissue and therewith side effects that might affect unintended organs and comorbid tissue that reciprocally stress the heart. The confinement of immunosuppressant uptake to the target organ reduces its graft versus host potential, increases its susceptibility to microchimerization by the host, and leaves the rest of the host body immunocompetent.

Provided the immunosuppressant used has a reversal agent, any release from the graft organ of the immunosuppressant can be eliminated by the release of the reversal agent from a side-entry jacket positioned along the venous drainage of the graft organ. If necessary, still greater restriction to the target is obtained by placement about the fibrous outer layer of the organ of one or more patch-magnets as described in U.S. Pat. No. 11,389,171 entitled Integrated System for the Infixion and Retrieval of Implants. Using this mechanical approach, the drug, if bound to a water soluble superparamagnetic carrier, need not have a reversal agent.

Further to eliminate any continued travel through one or more veins of the graft organ is to prepare the drug, even one for which no reversal agent is available, for magnetic extraction from the veins of the organ whether native or a graft organ. The drug, bound to a water soluble superparamagnetic carrier, is drawn out of the vein by a permanent magnet equipped side-entry jacket positioned about the venous drainage ductus of the graft organ. The magnet is incorporated into the jacket as a concentric layer thereof, radially magnetized to exert its extractive field toward the long axis of the substrate ductus.

An immunosuppressant, for example, is then delivered in the form of a water soluble superparamagnetic carrier-bound nanoparticulate. Methods for definitively targeting drugs to an anatomical target whether directly pipeline-targeted or systemically dispersed are described in U.S. Pat. No. 11,389,171 entitled Integrated System for the Infixion and Retrieval of Implants. References pertaining to water-soluble superparamagnetic drug carriers are provided below in section 2b(1)(b)(b5), entitled Heterotopic Double Heart Metered Compound Vascular Bypass, or Switched-organ, Transplantation [and so on]

Although probable, whether this makes it possible for the patient to be given live attenuated, or weakened, vaccine, so that immunized, the rest of the body can fight off the infection, or immunocompromised so that the graft organ is selectively susceptible to infection, or is, but the infection would be obliterated by continuous circulation through it from the rest of the body warrants study. If necessary, the system can gradually or intermittently reduce the level of the immunosuppressant targeted to the graft organ in coordination with the release of the antimicrobial.

The variables include 1. The starting immune competence of the patient and therefore the strength of the response to the vaccine, or counter thereto. 2. The patient has a primary immunodeficiency disorder, or 3. Is elderly, 4. Frail, debilitated, or 5. Sick: 6. The dose and effectiveness of the vaccine; 7. Whether vaccination is accomplished before or after transplantation; in an infant, 8. Whether maternal antibody protection has disappeared: 9. Whether the infection would be systemic if secondarily affecting certain organs such as yellow fever (Flavivirus), which secondarily and differentially involves the liver and kidneys, chickenpox (varicella-zoster herpesvirus/Human alphaherpesvirus 3), or measles (Measles morbillivirus), mumps (Mumps orthorubulavirus), which differentially affects the parotid glands, and rubella (Rubella virus), commonly immunized against together with combined vaccine; or would selectively and primarily affect a particular organ, such as the liver by hepatitis, and 10. Whether the organ sought out is that transplanted.

Another benefit of the transplant as an isolated target is the ability to selectively and/or differentially target hematopoietic or mesenchymal stem cells, regulatory T cells, or gene-modified regulatory T cells, for example, to the graft organ, and/or to the rest of the body with or without an immunosuppressant where these are prepared as magnetized and/or electrified nanoparticles with uptake, as described in U.S. Pat. No. 11,389,171, entitled Integrated System for the Infixion and Retrieval of Implants, increased with the aid of patch-magnets, for example (see, for example, Brasile, L., Henry, N., Orlando, G., and Stubenitsky, B. 2019. “Potentiating Renal Regeneration Using Mesenchymal Stem Cells,” Transplantation 103(2): 307-313; Grigorian Shamagian, L., Madonna, R., Taylor, D., Climent, A. M., Prosper. F., and 7 others 2019. “Perspectives on Directions and Priorities for Future Preclinical Studies in Regenerative Medicine,” Circulation Research 2019 124(6): 938-951; Todryk, S., Jozwik, A., de Havilland, J., and Hester, J. 2019. “Emerging Cellular Therapies: T Cells and Beyond,” Cells 8(3). pii: E284; Romano, M., Fanelli, G., Albany, C. J., Giganti, G., and Lombardi, G. 2019. “Past, Present, and Future of Regulatory T Cell Therapy in Transplantation and Autoimmunity,” Online, Frontiers in Immunology (Lausanne, Switzerland) 10:43; Zhang, Y., Fan, W., Wang, K., Wei. H., Zhang. R., and Wu. Y. 2019. “Novel Preparation of Au Nanoparticles Loaded Laponite Nanoparticles/ECM [extracellular matrix] Injectable Hydrogel on Cardiac Differentiation of Resident Cardiac Stem Cells to Cardiomyocytes,” Journal of Photochemistry and Photobiology, B, Biology 192:49-54; Dawson, N. A., Lamarche, C., Hoeppli, R. E., Bergqvist, P., Fung. V. C., and 8 others 2019. “Systematic Testing and Specificity Mapping of Alloantigen-specific Chimeric Antigen Receptors in Regulatory T Cells,” Journal of Clinical Investigation Insight 4(6). pii: 123672; Boroughs, A. C., Larson, R. C., Choi, B. D., Bouffard, A. A., Riley, L. S., and 7 others 2019. “Chimeric Antigen Receptor Costimulation Domains Modulate Human Regulatory T Cell Function,” Journal of Clinical Investigation Insight 5. pii: 126194; Gorain, B., Choudhury, H., Pandey, M., Kesharwani, P., Abeer, M. M., Tekade, R. K., and Hussain, Z. 2018. “Carbon Nanotube Scaffolds as Emerging Nanoplatform for Myocardial Tissue Regeneration: A Review of Recent Developments and Therapeutic Implications,” Biomedicine and Pharmacotherapy 104:496-508; Liu, N., Chen, J., Zhuang. J., and Zhu, P. 2018. “Fabrication of Engineered Nanoparticles on Biological Macromolecular (PEGylated Chitosan) Composite for Bio-active Hydrogel System in Cardiac Repair Applications,” International Journal of Biological Macromolecules 117:553-558; Chae, W. J. and Bothwell, A. L. M. 2018. “Therapeutic Potential of Gene-modified Regulatory T Cells: From Bench to Bedside,” Online, Frontiers in Immunology (Lausanne, Switzerland) 9:303; Lamarche, C. and Levings, M. K. 2018. “Guiding Regulatory T Cells to the Allograft,” Current Opinion in Organ Transplantation 23(1): 106-113; Zhang, Q., Lu, W., Liang, C, L., Chen, Y., Liu, H., Qiu, F., and Dai, Z. 2018. “Chimeric Antigen Receptor (CAR) Treg: A Promising Approach to Inducing Immunological Tolerance,” Online, Frontiers in Immunology (Lausanne, Switzerland) 9:2359; Di Franco, S., Amarelli, C., Montalto, A., Loforte, A., and Musumeci, F. 2018. “Biomaterials and Heart Recovery: Cardiac Repair, Regeneration and Healing in the MCS [mechanical circulatory support] Era: A State of the “Heart,” Journal of Thoracic Disease 10 (Supplement 20): S2346-S2362; Sicard, A., Levings, M. K., and Scott, D. W. 2018. “Engineering Therapeutic T Cells to Suppress Alloimmune Responses Using TCRs, CARs, or BARs [T cell receptors, single-chain chimeric antigen receptors, or antigen domains recognized by B cell receptors],” American Journal of Transplantation 18(6): 1305-1311; Kenry, N F N, Lee, W. C., Loh, K. P., and Lim, C. T. 2018. “When Stem Cells Meet Graphene: Opportunities and Challenges in Regenerative Medicine,” Biomaterials 155: 236-250; Kumar, S., Wang, J., Thomson, A. W., and Gandhi, C, R. 2017. “Hepatic Stellate Cells Increase the Immunosuppressant Function of Natural Foxp3+ [forkhead box P3/scurfin] Regulatory T Cells via IDO [indoleamine dioxygenase]-induced AhR [aryl hydrocarbon receptor] Activation,” Journal of Leukocyte Biology 101(2): 429-438; Narayanan, K., Mishra, S., Singh, S., Pei. M., Gulyas. B., and Padmanabhan, P. 2017. “Engineering Concepts in Stem Cell Research,” Biotechnology Journal 12(12); Frey, B. M., Zeisberger, S. M., and Hoerstrup, S. P. 2016. “Stem Cell Factories—The Rebirth of Tissue Engineering and Regenerative Medicine,” Transfusion Medicine and Hemotherapy 43(4): 244-246; Baei, P., Jalili-Firoozinezhad, S., Rajabi-Zeleti. S., Tafazzoli-Shadpour, M., Baharvand, H., and Aghdami, N. 2016. “Electrically Conductive Gold Nanoparticle-chitosan Thermosensitive Hydrogels for Cardiac Tissue Engineering,” Materials Science and Engineering, C. Materials for Biological Application 63:131-141; Mallone, A., Weber, B., and Hoerstrup, S. P. 2016. “Cardiovascular Regenerative Technologies: Update and Future Outlook,” Transfusion Medicine and Hemotherapy 43(4): 291-296; Mao, A. S. and Mooney, D. J. 2015. “Regenerative Medicine: Current Therapies and Future Directions,” Online, Proceedings of the National Academy of Sciences of the United States of America 112(47): 14452-14459; Benderitter, M., Caviggioli, F., Chapel, A., Coppes, R. P., Guha. C., and 6 others 2014. “Stem Cell Therapies for the Treatment of Radiation-induced Normal Tissue Side Effects,” Antioxidants and Redox Signaling (Larchmont, New York) 21(2): 338-355; Chapman, N. M. and Chi, H. 2014. “mTOR [mammalian target of rapamycin] Signaling. Tregs and Immune Modulation,” Immunotherapy 6(12): 1295-1311; Baum, C. E., Mierzejewska, B., Schroder, P. M., Khattar, M., and Stepkowski, S. 2013. “Optimizing the Use of Regulatory T Cells in Allotransplantation: Recent Advances and Future Perspectives,” Expert Review of Clinical Immunology 9(12): 1303-1314; Schmetterer, K. G., Neunkirchner, A., and Pickl, W, F. 2012. “Naturally Occurring Regulatory T Cells: Markers, Mechanisms, and Manipulation,” Federation of American Societies for Experimental Biology Journal 26(6): 2253-2276; Dangi, A., Sumpter. T, L., Kimura, S., Stolz, D. B., Murase, N., and 5 others 2012. “Selective Expansion of Allogeneic Regulatory T Cells by Hepatic Stellate Cells: Role of Endotoxin and Implications for Allograft Tolerance,” Journal of Immunology (Baltimore, Maryland) 188(8): 3667-3677; Shah, V. K. and Shali, K. K. 2009. “Regeneration of Myocardium-Dawn of a New Era!,” Journal of the Association of Physicians of India 57:312-24, 329-331; Allan, S. E., Broady, R., Gregori, S., Himmel, M. E., Locke, N., and 3 others 2008. “CD4+ T-regulatory Cells: Toward Therapy for Human Diseases,” Immunological Reviews 223:391-421).

By placing the patient on cardiopulmonary support or membrane oxygenation, and thus sustaining perfusion past death, damage to the organs associated with death and delay prior to revitalization can be avoided. Sustainment by replacement of the native with an artificial heart is discouraged as severely damaging to the heart removed as one of the organs most frequently needed for transplantation. In most instances, death occurs away from a medical facility, making the continuation of circulation past death unavoidable; however, a large proportion of patients do expire in a hospital at a time when the knowledge and apparatus required for preserving the body for organ donation are known and available throughout the developed world.

Accordingly, unless the irreversibly comatose patient dies while on life support so that the organs can be harvested while life support is continued, the use of his organs for transplantation should be discounted. Fortunately, however, patients in an irreversibly comatose state represent a tiny minority of in-hospital deaths. This is so even though the contraction of the heart is controlled by the intracardiac conduction system or cardionector, so that the heart may continue to beat for several days following death, but irregularly, with no assurance that the tissues of the body had been sufficiently oxygenated.

As indicated above, a cardinal factor in preserving organs for transplantation is that contrary to convention, life support with full body circulation sustained through the use of a defibrillator and spontaneous breathing should not be stopped following brain death-convention is precisely wrong in doing so. Most literature on the maximum duration of respiration and circulation by extracorporeal membrane oxygenation concerns survival and the postprocedural condition of the patient once taken off, or “weaned,” from the apparatus, not the maximum time a patient having been placed on life support while still alive can be preserved after death.

A given patient having died due to any number of causes, here the object is to initiate life support before and sustain perfusion past death as long as possible to allow switch transplantation of the solid organs with zero ischemic time and zero reperfusion injury. Damage to blood components over this term as a factor militating against longer duration is ameliorable through transfusion. Maintaining the patient always the primary consideration, here the question is less how long one can survive on life support than how best to prepare the patient for organ donation, and taken up below in this section, how long past death the body can be successfully perfused.

The patient will generally be best prepared for donation if intensive care is maintained past death. If the next of kin wish a severely brain injured relative to be removed from life support, then intensive care should be suspended only for the pronouncement of death, and immediately reinstituted upon the pronouncement of death. The deceased should be sustained as close to living as possible. Body systems interdependent, neither should disproportionate emphasis be placed upon the sustainment of selected organs slated for transplantation; even though no gland is to be transplanted endocrine support, for example, should be continued.

If the lungs are to be used, ‘normal’ breathing,” (normal respiration, spontaneous ventilation) should be continued through direct brainstem stimulation with or without the aid of a surgical chest dome, addressed above in this section and below in sections 2b(2)(k), entitled Surgical Chest Dome, and in section 6, entitled Description of the Preferred Embodiments of the Invention with mechanical ventilation kept to a minimum, preferably through the use of high-flow nasal cannula oxygen therapy, addressed in section 2b(2)(g), entitled Ischemia and hypoperfusion-free carotid endarterectomy bypass and prosthesis (Shetty, V, L., Mali, S. S., Shetty, S. V., and Shinde, P, D. 2017. Op cit. in this section) (see, also, for example, Beckman, E. J. 2019. “Management of the Pediatric Organ Donor,” Journal of Pediatric Pharmacology and Therapeutics 24(4): 276-289; Kieslichova, E., Protus, M., Nemcova, D., and Uchytilova, E. 2019. “Single Multidrug Resistant Enterobacteriacae Donor-derived Infection in Four Solid Organ Transplant Recipients: A Case Report,” Online, BioMed Central Surgery 19(1): 111; Martinez-Soba, F., Minambres, E., Martinez-Camarero, L., Arlaban-Carpintero, M., Moreno-Ortigosa, J. A., Calleja-Muñoz, V., and Ballesteros, M, A. 2019. “Results after Implementing a Program of Intensive Care to Facilitate Organ Donation,” Transplantation Proceedings 51(2): 299-302; Martinez-Soba, F., Perez-Villares, J. M., Martinez-Camarero, L., Lara, R., Monzon, J, L., and 4 others 2019. “Intensive Care to Facilitate Organ Donation: A Report on the Experience of 2 Spanish Centers with a Common Protocol,” Transplantation 103(3): 558-564; Ream, R. S., Clark, M. G., and Armbrecht, E. S. 2019. “Pediatric Donor Management Goals in Use by US Organ Procurement Organizations,” Progress in Transplantation (Aliso Viejo, California) 29(2): 150-156; Zirpe, K. and Gurav. S. 2019. “Brain Death and Management of Potential Organ Donor: An Indian Perspective,” Indian Journal of Critical Care Medicine 23 (Supplement 2): S151-S156; Dominguez-Gil. B., Coll. E., Pont. T., Lebron, M., Miñambres. E., and 7 others 2017. “End-of-life Practices in Patients with Devastating Brain Injury in Spain: Implications for Organ Donation,” Medicina Intensiva (Madrid, Spain) 41(3): 162-173; Dominguez-Gil. B., Coll. E., Elizalde, J., Herrero, J. E., Pont. T., and 11 others 2017. “Expanding the Donor Pool through Intensive Care to Facilitate Organ Donation: Results of a Spanish Multicenter Study,” Transplantation 101(8): e265-e272; Kumar, L. 2016, Op cit.; Kotloff, R. M., Blosser, S., Fulda, G. J., Malinoski, D., Ahya, V. N., and 37 others 2015. “Management of the Potential Organ Donor in the ICU: Society of Critical Care Medicine/American College of Chest Physicians/Association of Organ Procurement Organizations Consensus Statement,” Critical Care Medicine 43(6): 1291-1325; Youn, T. S. and Greer, D. M. 2014. “Brain Death and Management of a Potential Organ Donor in the Intensive Care Unit,” Critical Care Clinics 30(4): 813-831).

For the purpose of preserving a deceased patient having been supported by extracorporeal membrane oxygenation past brain death in order to transplant the organs by sudden compound vascular bypass, or sudden switched-organ transplantation and thus avoid reperfusion injury and organ degradation in a large center, a period of days will sustain the donor long enough for the operation. However, the longer circulation and respiration can be sustained, the greater will be the opportunity to transplant the organs, and the longer can be the immune tolerance induction during metered compound vascular bypass, or sudden switched-organ transplantation.

Where survival is concerned, beyond the first week or two of support, current venoatrial extracorporeal membrane oxygenation for patients with cardiogenic shock, even with the aid of a left ventricular assist device, is of limited dependability. Unless the condition of ventricular dysfunction is the result of a repairable defect, such as a faulty valve or ventriculoseptal defect, such a heart is unsuitable for transplantation. Indeed, where repair is dependable, such hearts, not having become impaired due to a myocardial infarction, cardiomyopathy, coronary atherosclerosis, or sudden decompression, for example, may be considered for transplantation using a switch method.

Otherwise, the other organs sustained on life support past death should be usable (see, for example, Chung. M., Zhao, Y., Strom, J. B., Shen, C., and Yeh, R. W. 2019. “Extracorporeal Membrane Oxygenation Use in Cardiogenic Shock: Impact of Age on In-hospital Mortality, Length of Stay, and Costs,” Critical Care Medicine 47(3): e214-e221; Wang, L., Yang, F., Wang, X., Xie, H., Fan, E., and 4 others 2019. “Predicting Mortality in Patients Undergoing VA-ECMO [venoatrial extracorporeal membrane oxygenation] after Coronary Artery Bypass Grafting: the REMEMBER [Reducing Early Mortality and Early Morbidity by Empiric Tuberculosis Treatment Regimens] Score,” Critical Care (London, England) 23(1): 11; Charon, C., Allyn, J., Bouchet, B., Nativel, F., Braunberger, E., and 3 others 2018. “Ten Thousand Kilometre Transfer of Cardiogenic Shock Patients on Venoarterial Extracorporeal Membrane Oxygenation for Emergency Heart Transplantation: Cooperation between Reunion Island and Metropolitan France,” European Heart Journal Acute Cardiovascular Care 7(4): 371-378; El Sibai, R., Bachir, R., El Sayed, M. 2018. “ECMO Use and Mortality in Adult Patients with Cardiogenic Shock: A Retrospective Observational Study in U.S. Hospitals,” Online, BioMed Central Emergency Medicine 18(1): 20; El Sibai, R., Bachir, R., and El Sayed, M. 2018. “Outcomes in Cardiogenic Shock Patients with Extracorporeal Membrane Oxygenation Use: A Matched Cohort Study in Hospitals across the United States,” Online, BioMed Research International 2018:2428648; Smith, M., Vukomanovic, A., Brodie, D., Thiagarajan, R., Rycus, P., and Buscher, H. 2017. “Duration of Veno-arterial Extracorporeal Life Support (VA ECMO) and Outcome: An Analysis of the Extracorporeal Life Support Organization (ELSO) Registry,” Critical Care (London, England) 21(1): 45).

Mechanical circulatory support subject to lung injury, pneumonia, and time-limited, to allow prospective donors to remain usable for a longer period and prompt the establishment of centralized medical center storage depots, a more spontaneous means for obtaining sustained cardiorespiratory function without these complications would prove beneficial (See Hoe, L. E., Bartnikowski, N., Wells. M. A., Suen, J. Y., and Fraser. J, F. 2019. “Hurdles to Cardioprotection in the Critically Ill,” International Journal of Molecular Science 20(15). pi: E3823; Hsieh, A., Tumin, D., McConnell, P. I., Galantowicz, M., Tobias, J. D., and Hayes, D. Jr. 2017. “Influence of Transplant Center Procedural Volume on Survival Outcomes of Heart Transplantation for Children Bridged with Mechanical Circulatory Support,” Pediatric Cardiology 38(2): 280-288).

In large centers with neurological specialists, anesthesiologists, and intensivists, an attempt can be made to eliminate complete and extended dependency upon extracorporeal membrane oxygenation for the sustainment of cardiorespiratory function. A decompressive craniectomy to preempt the increase in intracranial pressure that will ensue upon death is performed while the prospective donor remains on life support. Nutritional, endocrine, body temperature, blood electrolyte balance, and so on for maintaining a deceased is addressed below in this section. The control of respiration and circulation are intimately interlocked centrally and peripherally. A conventional ventilator allows continued inhalation (inspiration) and exhalation (expiration) past death while the deceased remains on life support.

Infection and deterioration are warded off throughout intact preservation, transfer to the recipient, and postoperatively by incorporating antimicrobials, hormones, and other essential substances likely to have become depleted by infusion into the donor, which is then communicated to the recipient during switch transplantation. If the donor has been implanted with an automatic disorder response system, the detection of shortages in essential substances and the release thereof directly into the vascular tree through ductus side-entry jackets can proceed autonomously with intermittent checking by a specialist.

Unlike respiration, the control of heartrate is internal to the heart, the control center in the medulla oblongata applying adjustments thereto as necessitated by exertion, emotional or physical stress, or changes in pressure or acidity of the blood. Direct stimulation of the circulatory center in the medulla is, however, a means for adjusting rate or stroke volume, best accomplished through the direct targeting of epinephrine or norepinephrine directly to the brain through a ductus side-entry jacket as addressed below in section 2d, entitled Body Surface Side-entry Ports. For the most part, control over its functioning can be accomplished locally to the heart.

The continuation of brainstem and parasympathetic innervation of the sinoatrial node past death of the higher brain, primarily by the medullary center through the vagus nerve, and its peripheral innervation by sympathetic nerves is thus avoidable; that is, in contrast to respiratory control, access to the central brainstem and autonomic control over circulation should be unnecessary. Neural rather than chemical in discharging command signals to the muscles of the chest, control of respiration is best accomplished by direct electrical, chemical, or optogenetic stimulation, or electrical neuromodulation of the respiratory centers in the brainstem.

Direct stimulation of the cardiovascular control centers in the brainstem, or secondarily the kidneys, are options, but accomplished more simply through the direct targeting of cardioactive substances, such as epinephrine, norepinephrine, antidiuretic hormone, atrial natriuretic peptide, nitrates, or nicotine, directly to the heart, as described and illustrated in FIG. 16, in U.S. Pat. No. 11,759,186, entitled Ductus Side-entry Jackets and Prosthetic Disorder Response Systems.

Much information concerning the pneumotaxic and apneustic centers of the pontine respiratory group, and the dorsal and ventral respiratory groups in the medulla, has already been elucidated (see, for example, Baertsch, N, A. and Ramirez, J.-M. 2019. “Insights into the Dynamic Control of Breathing Revealed through Cell-type-specific Responses to Substance P,” e-Life 8: e51350; Baertsch, N. A., Severs, L. J., Anderson, T. M., and Ramirez, J.-M. 2019. “A Spatially Dynamic Network Underlies the Generation of Inspiratory Behaviors,” Online, Proceedings of the National Academy of Sciences of the United States of America 116(15): 7493-7502; Ben-Tal, A., Wang, Y., and Leite, M. C. A. 2019. “The Logic behind Neural Control of Breathing Pattern,” (Nature Research) Scientific Reports 9(1): 9078; Dhingra, R. R., Furuya, W. I., Bautista, T. G., Dick, T. E., Galan, R. F., and Dutschmann, M. 2019. “Increasing Local Excitability of Brainstem Respiratory Nuclei Reveals a Distributed Network Underlying Respiratory Motor Pattern Formation,” Online, Frontiers in Physiology 10:887; Kaur, S. and Saper, C, B. 2019. “Neural Circuitry Underlying Waking Up to Hypercapnia,” Online, Frontiers in Neuroscience 13:401; Rubin, J, E. and Smith, J, C. 2019. “Robustness of Respiratory Rhythm Generation across Dynamic Regimes,” Online, PLoS [Public Library of Science] Computational Biology 15(7): e1006860; Ausborn, J., Koizumi, H., Barnett, W. H., John, T. T., Zhang, R. and 3 others 2018. “Organization of the Core Respiratory Network: Insights from Optogenetic and Modeling Studies,” Online, PLoS [Public Library of Science] Computational Biology 14(4): e1006148; Del Negro, C. A., Funk, G. D., and Feldman, J. L. 2018. “Breathing Matters,” Nature Reviews. Neuroscience 19(6): 351-367; Guyenet, P. G., Stornetta, R, L., Souza, G. M. P. R., Abbott, S, B. G., Shi, Y., and Bayliss, D. A, 2019. “The Retrotrapezoid Nucleus: Central Chemoreceptor and Regulator of Breathing Automaticity,” Trends in Neurosciences 42(11): 807-824; Rubin, J, E. and Smith, J, C. 2019. “Robustness of Respiratory Rhythm Generation across Dynamic Regimes,” Online, PLoS [Public Library of Science] Computational Biology, at doi.org/10.1371/journal.pcbi.1006860; Mulkey, S, B. and Plessis, A, D. 2018. “The Critical Role of the Central Autonomic Nervous System in Fetal-Neonatal Transition,” Seminars in Pediatric Neurology 28:29-37; Ramirez, J.-M. and Baertsch, N, A. 2018. “The Dynamic Basis of Respiratory Rhythm Generation: One Breath at a Time,” Annual Review of Neuroscience 41:475-499; Ramirez, J.-M. and Baertsch, N. 2018. “Defining the Rhythmogenic Elements of Mammalian Breathing,” Physiology (Bethesda, Maryland) 33(5): 302-316; Vann, N. C., Pham, F. D., Dorst, K. E., and Del Negro, C, A. 2018. “Dbx1 [Developing Brain homeobo.X 1 protein coding gene] Pre-Botzinger Complex Interneurons Comprise the Core Inspiratory Oscillator for Breathing in Unanesthetized Adult Mice,” eNeuro (Washington, D.C.) 2018 5(3): ENEURO.0130-18; Akins, V. T., Weragalaarachchi, K., Picardo, N, C. D., Ann L. Del Negro, R., and Del Negro, C, A. 2017. “Morphology of Dbx1 [Developing Brain homeobo.X 1 protein coding gene] Respiratory Neurons in the PreBotzinger Complex and Reticular Formation of Neonatal Mice,” Nature Scientific Data 4:170097, Online at nature.com/articles/sdata201797; Ikeda, K., Kawakami, K., Onimaru, H., Okada, Y., Yokota, S., and 4 others 2017. “The Respiratory Control Mechanisms in the Brainstem and Spinal Cord: Integrative Views of the Neuroanatomy and Neurophysiology,” Journal of Physiological Sciences 67(1): 45-62; Russo, M. A., Santarelli, D. M., and O'Rourke, D. 2017. “The Physiological Effects of Slow Breathing in the Healthy Human,” Breathe (Sheffield, England) 13(4): 298-309, FIG. 2 showing coupling between respiratory and cardiovascular control in cardiorespiratory control; Basiago, A. and Binder, D. K. 2016. “Effects of Deep Brain Stimulation on Autonomic Function,” Brain Sciences 6(3). pii: E33; Song, H., Hayes, J. A., Vann, N. C., Wang, X., LaMar, M. D., and Del Negro, C, A. 2016. “Functional Interactions between Mammalian Respiratory Rhythmogenic and Premotor Circuitry,” Journal of Neuroscience 36(27): 7223-7233; Guyenet, P, G. and Bayliss, D, A. 2015. “Neural Control of Breathing and CO2 Homeostasis,” Neuron 87(5): 946-961; Lovick, T. 2014. “Deep Brain Stimulation and Autonomic Control,” Experimental Physiology 99(2): 320-325; Safonov, V, A. and Tarasova, N. N. 2010. “Effects of Electrical Stimulation of Respiratory Center in the Event of Termination of Its natural Rhythmic Activity,” Bulletin of Experimental Biology and Medicine 149(4): 397-399; Van de Louw, A., Médigue, C., Papelier, Y., Landrain, M., and Cottin, F. 2010. “Role of Brainstem Centers in Cardiorespiratory Phase Difference during Mechanical Ventilation,” Respiration Physiology and Neurobiology 174(1-2): 119-127; Guyenet, P, G. 2008. “The 2008 Carl Ludwig Lecture: Retrotrapezoid Nucleus, CO2 Homeostasis, and Breathing Automaticity,” Journal of Applied Physiology (Bethesda, Maryland) 105(2): 404-416; Come, S. and Bshouty, Z. 2005. “Basic Principles of Control of Breathing,” Respiratory Care Clinics of North America 11(2): 147-172; Safonov, V. A., Eksler, N. D., and Pelevinov, V, A. 1983. “Formation of Respiratory System Function in Ontogeny. I. Intrauterine Respiratory Movements,” in Russian with abstract at Pubmed), Nauchnye Doklady Vysshel Shkoly. Biologicheskie Nauki [Scientific Reports of the Advanced School. Biological Sciences] (12): 4-22; Berger, A. J., Mitchell, R. A., and Severinghaus, J. W. 1977. “Regulation of Respiration: (Second of Three Parts),” New England Journal of Medicine 1977 297(3): 138-143; Kalia, M. 1977. “Neuroanatomical Organization of the Respiratory Centers,” Federation Proceedings 1977 36(10): 2405-2411; Safonov, V, A. 1977. “Organization of the Respiratory Center. II. Responses of Respiratory Neurons. Modeling the Respiratory Center,” (in Russian with abstract at Pubmed), Nauchnye Doklady Vysshel Shkoly. Biologicheskie Nauki [Scientific Reports of the Advanced School. Biological Sciences] (3): 7-20; Mitchell, R, A. and Berger, A. J. 1975. “Neural Regulation of Respiration,” American Review of Respiratory Disease 111(2): 206-224).

Cardiopulmonary bypass support and even extracorporeal membrane oxygenation is damaging to blood cells, so that even on extracorporeal membrane oxygenation with blood transfusion, artificial sustainment thus, while considerable, lasting for weeks, may ultimately be limited. Mechanical ventilation administered by a skilled practitioner does not damage blood cells but if not correctly adjusted can damage the lungs, and sores about the mouth from the mask or injury to the trachea from the endotracheal tube are other frequent complications. For lung transplantation in particular, it is preferable that circulation and to the extent possible, spontaneous respiration be preserved.

Spontaneous control over respiration by the brainstem sensory center responsive to exertion, altitude, emotional extremes, and temperature is bypassed by direct stimulation of the pontine and medullary dorsal and ventral respiratory groups by means of stereotactically positioned needle electrodes and/or to the extent that the dissipation of the higher temperature it emits will allow, the future application of optogenetics. Still early in development, optogenetics probes are positioned atop the skull to selectively illuminate light-engineered responsive cell types within the brain.

The optic fiber is encircled by an optic canula, which for angular stabilization, is affixed to the skull with ‘cranioplastic cement,’ Such an arrangement is satisfactory for experimentation with rats over a limited time, but not for permanent implantation in human patients to provide long-term if not lifelong therapy. Permanent fixation with adhesives inside the body is unsatisfactory for several reasons, to include hydrolytic and enzymatic breakdown, secretion into the interface, inadequate strength, and adverse tissue reactions. Optogenetics has the potential to suppress infection, a frequent cause for graft organ failure. Following kidney transplantation, for example, optogenetics can also be used to stimulate the pelvis to initiate peristalsis and protect the kidney and bladder against infection.

Adhesives used internally are meant to maintain incisional or fracture interfaces in juxtaposed relation long enough to allow healing by first intention. However, in the future, numerous type probes, diagnostic and therapeutic, of generally styloid confirmation will require to be angle-stabilized to maintain constancy of aim. For this reason, U.S. Pat. No. 11,013,858, entitled Nonjacketing Side-entry Connectors and Prosthetic Disorder Response Systems, describes means for the permanent stable angular fixation of generally styloid shaped diagnostic and therapeutic implants to maintain their aim at a target within an organ parenchyma, for example. For optogenetic therapy, the styloid implant is the light source. These use no adhesives, and incorporate means for dispelling adverse tissue reactions.

Initially directed to the ability to selectively control neurons and neuronal circuits in the brain, additional areas of medical practice for which the fixation of optogenetic probes will be essential include noninjurious resynchronization of the heart, sustaining a deceased prospective organ donor beyond the term afforded by extracorporeal membrane oxygenation, the intentional use of heat for therapeutic purposes, and stimulation along the urinary tract, primarily of the bladder.

Applications of optogenetics are addressed in this and the sections respective of different graft organs transplanted using the switch method. The wider purpose in providing these reliably anchoring connectors is to enable the implementation of implanted automatic disorder response systems capable of providing targeted diagnostic and therapeutic function inside the body indefinitely without the need for surgical reentry. Service thus requires the ability to stabilize diagnostic probes, such as a miniature fiberscope, angioscope, or intravascular ultrasound probe, and/or a therapeutic device, such as a catheter with hollow needle for the positionally targeted release of medication, or the aimed application of electrical or light stimulation as stipulated by the implanted disorder response system controller.

The anchoring needles of nonjacketing side-entry connectors can be electrified to deliver electrical current among the needles in any pattern, such as between the needles of a single connector or between needles of separate connectors, can be hollow to allow injection, and radiation shielded to allow the direct pipeline targeting of moderate dose rate drugs, and these modalities can be used in combination. Conventional injection is through the beveled tips of the needle ejection lumina, but can also be through apertures along the sides of the needles.

Using concurrent unilateral or bilateral injection and passing tissue surface-coplanar side-to-side electrical current between the needles of one or separate connectors can be used for electrophoresis, electromigration, and electro-osmosis with increased tissue permeability, hence deeper drug penetration-effectively, the intracorporeal administration of a drug through noncutaneous, or deep tissue, iontophoresis. Intracorporeal tissue iontophoresis is currently unseen due to the lack of essential apparatus and because the action must take place inside the body. A fully implanted disorder control system requires only to have the microcontroller, or in a comorbidity capable system, the master control microprocessor, loaded with the required subroutine. U.S. Pat. No. 11,013,858 describes and illustrates jackets shielded to allow the delivery of low to moderate dose rate radioactive substances.

The drug reservoirs and lines must be shielded thus as well. The medication is delivered to the anchoring needles through the connector accessory channel or channels just as specified for drug delivery using side-entry jackets on the blood supplies. An important difference is that whereas delivery through the blood supply through side-entry jackets completely perfuses throughout the organ, delivery at the organ surface is targeted not just to the organ but a lesion in a subportion thereof—that is, the targeting is more highly defined. That the placement of nonjacketing side-entry connectors is through insertion through small incisions is invasive is another instance where it is argued that proper treatment at the outset which allows ‘remote control’ ever after is ultimately far superior to any alternative.

Restricted to cutaneous application, a ‘step up’ from a transdermal patch for access to the bloodstream with noninvasiveness the benefit, the potential for directly targeting tissue inside the body, such as to release into restrict an immunosuppressant into the lesions of an inflammatory bowel disease, thus avoiding side effects elsewhere is precluded. Means that deliver drugs into the circulation depend upon an intrinsic affiliation or functional relation of the drug to the intended target, such as the relation of iodine to the thyroid gland or of sex hormones to the gonads. In contrast, direct mechanical delivery of substances into the substrate tissue achieves markedly superior targeting to spare untargeted tissue exposure to the substrance; the side effects of chemotherapeutics and steroids, for example, clearly warrant restricted targeting. Iontophoresis also has diagnostic potential (see, for example, Giri, T. K., Ghosh, B., Bose, P., Saha, S., and Sarkar, A. 2019. “Extraction of Levetiracetam for Therapeutic Drug Monitoring by Transdermal Reverse Iontophoresis,” European Journal of Pharmaceutical Sciences 128:54-60; Giri, T. K., Chakrabarty, S., and Ghosh, B. 2017. “Transdermal Reverse Iontophoresis: A Novel Technique for Therapeutic Drug Monitoring,” Journal of Controlled Release 246: 30-38; Ching, C. T., Fu. L. S., Sun, T. P., Hsu. T. H., and Chang, K. M. 2012. “Use of Electroporation and Reverse Iontophoresis for Extraction of Transdermal Multibiomarkers,” International Journal of Nanomedicine (Auckland, New Zealand) 7:885-894; Sieg, A. and Wascotte. V. 2009. “Diagnostic and Therapeutic Applications of Iontophoresis,” Journal of Drug Targeting (London, England) 17(9): 690-700). Applied intracorporeally, suitable sensors and removal means are also required.

Conventional iontophoresis remains limited to cutaneous delivery (see, for example, Massella, D., Argenziano, M., Ferri. A., Guan, J., Giraud. S., and 3 others 2019. “Bio-functional Textiles: Combining Pharmaceutical Nanocarriers with Fibrous Materials for Innovative Dermatological Therapies,” Pharmaceutics (Basel, Switzerland) 11(8). pii: E403; Park, J., Lee, H., Lim, G. S., Kim, N., Kim, D., and Kim, Y, C. 2019. “Enhanced Transdermal Drug Delivery by Sonophoresis and Simultaneous Application of Sonophoresis and Iontophoresis,” American Association of Pharmaceutical Scientists PharmSciTech 20(3): 96; Chakrabarty, S., Ghosh, B., Giri, T. K., and Khanam, J. 2018. “Reverse Iontophoretic Extraction of Gabapentin: A Mechanistic Study,” Current Drug Delivery 15(7): 965-971; Karpinski, T. M. 2018. “Selected Medicines Used in Iontophoresis,” Pharmaceutics (Basel, Switzerland) 10(4). pii: E204; Korsten, M. A., Lyons, B, L., Radulovic, M., Cummings, T. M., Sikka, G., and 5 others 2018. “Delivery of Neostigmine and Glycopyrrolate by Iontophoresis: A Nonrandomized Study in Individuals with Spinal Cord Injury,” Spinal Cord 56(3): 212-217; Kubota, K., Miyamoto, T., Inoue. T., and Fukayama, H. 2018. “Alternating Current Iontophoresis for Control of Postoperative Pain,” Anesthesia Progress 2018 65(2): 106-110; Nkemngu, N. J. and Tochie, J. N. 2018. “Atrio-ventricular Block following Neostigmine-Glycopyrrolate Reversal in Non-heart Transplant Patients: Case Report,” Anesthesia Progress 65(3): 187-191; Seah, B, C. and Teo, B. M. 2018. “Recent Advances in Ultrasound-based Transdermal Drug Delivery,” International Journal of Nanomedicine (Auckland, New Zealand) 13:7749-7763; Zhao, R., Wang, C., Lu, F., Du, L., Fang, Z., and 4 others 2018. “A Flexible Interdigital Electrode Used in Skin Penetration Promotion and Evaluation with Electroporation and Reverse Iontophoresis Synergistically,” Sensors (Basel, Switzerland) 18(5). pii: E1431; Dave, S., Shriyan, D., and Gujjar, P. 2017. “Newer Drug Delivery Systems in Anesthesia,” Journal of Anaesthesiology, Clinical Pharmacology 33(2): 157-163; Ita, K. 2016. “Transdermal Iontophoretic Drug Delivery: Advances and Challenges,” Journal of Drug Targeting 24(5): 386-391; Nair, A. B., Kumria, R., Al-Dhubiab, B. E., Attimarad, M., and Harsha, S. 2015. “Noninvasive Sampling of Gabapentin by Reverse Iontophoresis,” Pharmaceutical Research 32(4): 1417-1424; Roustit, M., Blaise, S., and Cracowski, J. L. 2014. “Trials and Tribulations of Skin Iontophoresis in Therapeutics,” British Journal of Clinical Pharmacology 77(1): 63-71; Dhote, V., Bhatnagar, P., Mishra, P. K., Mahajan, S. C., and Mishra, D. K. 2012. “Iontophoresis: A Potential Emergence of a Transdermal Drug Delivery System,” Scientia Pharmaceutica 80(1): 1-28).

Direct injection thus can be of any drug in fluid form into any tissue—the detrusor, bowel, rectum, or myocardium, for example, injection in accordance with the prescription-program automatically or in response to sensor input data or a strict schedule. Automatic direct intracorporeal drug or other fluid agent pipeline targeted thus has wide potential application in the treatment of both native and transplant organs. Following a metered switch double heart transplantation to add a second heart to that native or the replacement of the native by two hearts, the benefit of this capability is considerable.

The chemical alteration, rather than increased penetrability of drugs through the passage of electrical current upon ejection as in conventional iontophoresis, warrants further study. The design of nonjacketing side-entry connectors allows current to be passed between the needles on one side to those on the other in either direction according to any pattern of discharge (Craine A, Krishnamurthy A, Villongco C T, Vincent K, Krummen D E, and 5 others 2024. “Successful Cardiac Resynchronization Therapy Reduces Negative Septal Work in Patient-specific Models of Dyssynchronous Heart Failure,” Online, bioRxiv [in press] (Cold Spring Harbor, New York) 14:2024.05.13.593804, with update in 20(10): e1012150 PLoS [Public Library of Science] Computational Biology (San Francisco, California); Craine A, Krishnamurthy A, Villongco C T, Vincent K, Krummen D E, and 5 others 2024. “Successful Cardiac Resynchronization Therapy Reduces Negative Septal Work in Patient-specific Models of Dyssynchronous Heart Failure,” Online, PLoS [Public Library of Science] Computational Biology (San Francisco, California) 20(10): e1012150; Nelson, G. S., Berger, R. D., Fetics, B. J., Talbot, M., Spinelli, J. C., Hare, J. M., and Kass, D, A. 2000. “Left Ventricular or Biventricular Pacing Improves Cardiac Function at Diminished Energy Cost in Patients with Dilated Cardiomyopathy and Left Bundle-branch Block,” Online, Circulation (Hagerstown, Maryland) 102(25): 3053-3059).

In a deceased prospective organ donor maintained on intensive cardiorespiratory, endocrine, thermal, and other essential support as delineated by Shetty et al., for example (Shetty, V, L., Mali, S. S., Shetty, S. V., and Shinde, P, D. 2017, Op cit., this section) the effect is unknown as to the duration of preservation. Galvanism may facilitate the anastomosis of donor and recipient nerve endings, such as of the vagus, accelerans, or phrenic nerves (Ball, C. M. and Featherstone, P. J. 2019. “Medical Galvanism-A Prelude to Defibrillation,” Anaesthesia and Intensive Care 47(1): 4-6; Cardozo Pinto, D, F. and Lammel, S. 2019. “Hot Topic in Optogenetics: New Implications of in Vivo Tissue Heating,” Nature Neuroscience 22(7): 1039-1041; Krasnikov, I., Seteikin, A., and Roth, B. 2019. “Advances in the Simulation of Light-Tissue Interactions in Biomedical Engineering,” Biomedical Engineering Letters 9(3): 327-337; Owen, S. F., Liu, M. H., and Kreitzer, A, C. 2019. “Thermal Constraints on in Vivo Optogenetic Manipulations,” Nature Neuroscience 22(7): 1061-1065; Periyasamy, V. and Pramanik, M. 2017. “Advances in Monte Carlo Simulation for Light Propagation in Tissue,” Institute of Electrical and Elecronics Engineers Reviews in Biomedical Engineering 10:122-135; Yona, G., Meitav, N., Kahn, I., and Shoham, S. 2016. “Realistic Numerical and Analytical Modeling of Light Scattering in Brain Tissue for Optogenetic Applications,” eNeuro 3(1). pii: ENEURO.0059-15.2015; Stujenske, J. M., Spellman, T., and Gordon, J, A. 2015. “Modeling the Spatiotemporal Dynamics of Light and Heat Propagation for in Vivo Optogenetics,” Cell Reports 12(3): 525-534; Thompson, D. L. 1949. “Hydrogalvanism; a Modern Form of Medical Galvanism,” Journal of the National Medical Association 41(6): 261-263; Jackson E C, Seddon H J. 1945. “Galvanism and Denervated Muscle Atrophy. Influence of Galvanic Stimulation on Muscle Atrophy Resulting from Denervation,” Online, British Medical Journal 2(4423): 485-486).

Substantially normal circulation and respiration of the deceased donor encourage sustaining and harvesting of the heart and respiratory tract last. While subject to adverse side effects, the more so the longer such treatments are applied, processes such as hemodialysis, peritoneal dialysis, liver dialysis, affinity chromatography, and plasmapheresis will usually sustain the kidneys, liver, and most other organs and tissues of a deceased donor long enough so that these as well as the heart or portions of the respiratory tract will remain acceptable for transplantation without all the tissues of the body having become degraded due to the blood having been damaged through the use of cardiopulmonary bypass or extracorporeal membrane oxygenation, for example (see, for example, Evers, J. and Taborski, U. 2018. “Anticoagulation, Bleeding, and Clotting at Donor Plasmapheresis,” Journal of Clinical Apheresis 33(4): 538-540; Wiersum-Osselton, J. C., Marijt-van der Kreek, T., and van Dongen, R. 2013. “Plasmapheresis in Healthy Donors in the Netherlands: Cohort Study of Risk Factors for Donor Complications,” Transfusion and Apheresis Science 48(2): 163; Schulzki, T., Seidel, K., Storch, H., Karges, H., Kiessig, S., and 7 others 2006. “A Prospective Multicentre Study on the Safety of Long-term Intensive Plasmapheresis in Donors (SIPLA),” Vox Sanguinis 91(2): 162-173; Rossi, P, L., Cecchini, L., Menichella, G., De Rosa, G., Alfano, G., and 5 others 1991. “Comparison of the Side Effects of Therapeutic Cytapheresis and Those of other Types of Hemapheresis,” Haematologica 76 Supplement 1:75-80). The order in which organs are harvested is addressed below in section 2b(1)(b).

The potential applications for optogenetic as well as electrostimulatory technology pertinent to organ transplantation, control over urinary function, cessation of ventricular fibrillation, and the prevention of sudden arrest as addressed herein have additional potential in establishing the ability to sustain spontaneous ventilation, or ‘normal’ breathing,′ in a deceased patient for organ donation by direct stimulation of the motor or efferent respiratory centers in the brainstem. Such assumes that the donor has a competent diaphragm and lungs.

The preferability of ventilation through direct stimulation is based upon the numerous and serious complications associated with mechanical ventilation, some of no concern in a terminal patient supported for organ harvesting, but others of considerable concern, such as pneumothorax and pneumonia, citations to the literature provided in section 2b(2)(g) below, entitled Ischemia and hypoperfusion-free carotid endarterectomy bypass and prosthesis. If the diaphragm and/or lungs are not competent, and/or the process of direct stimulation remains in development, mechanical ventilation, which poses a significantly lessened risk of complications in a corpse, is used.

The application of direct stimulation to the brainstem respiratory centers should be initiated well before, with the object of interdicting, and detaining if not preventing, the final stage of ‘coma dépassé,’ or the complete cessation of vegetative activity, whereupon autophagy would begin to ruin the organs. Paradoxically, the application of these measures can be expected precisely to interfere with the ability to identify at exactly what point the donor would otherwise certainly die.

The object in imaging then, is to identify the earliest appearance of a degree in deterioration beyond which no matter how aggressively treated, the patient would certainly die. This, rather than a more advanced state of deterioration, is the time to harvest organs-when to do so would no longer impose any greater threat of death to the patient but endothelial function still remained intact, before ischemia fundamentally degraded graft organ viability. Accordingly, the taking of organs should commence pursuant not to a certification of death but rather to a certification that the patient had ceased to be recoverable.

Some background pertinent to the changes attendant upon death in the brain, hence, to preservation and the potential for the sustainment of spontaneous ventilation past death through direct stimulation of the respiratory and circulatory centers after death, for example, already exists. Death of the higher brain is premonitory for death of the brainstem but the transition is through stages of deepening coma until even the vegetative functions are abolished. Irreversible cessation of brainstem functions follows a loss of perfusion, of which the measures cited herein (see, for example, Shetty. V, L., Mali, S. S., Shetty, S. V., and Shinde, P, D. 2017. Op cit. in this section) should allow the avoidance.

The events preceding complete death of the brain have been studied using several means (Walter, U. and Brandt, S, A. 2019. “Diagnosis of Irreversible Loss of Brain Function (“Brain Death”)—What is New?,” (in German with English abstract at Pubmed), Nervenarzt (Neurologist, Berlin, Germany) 90(10): 1021-1030; Rastogi, P. 2016. “The Mystique of Brain Death,” Journal of Clinical and Experimental Hepatology 6(2): 79-80; Dhanwate, A, D. 2014. “Brainstem Death: A Comprehensive Review in Indian Perspective,” Indian Journal of Critical Care Medicine (New Delhi, India) 18(9): 596-605: Lang, C. J. 2011. “There is No Reversible Brain Death,” Critical Care Medicine 39(9): 2205-2206; Wijdicks, E. F., Varelas, P. N., Gronseth, G. S., and Greer, D. M. 2011. “There is No Reversible Brain Death,” Critical Care Medicine 39(9): 2204-2206; Goulon, M. and Goulon-Goeau, C. 1989. “Coma and Cerebral Death,” (in French with English abstract at Pubmed), Revue du Praticien (The Practitioner's Review, Paris, France) 39(27): 2428-2433; Harvard Medical School 1984. “Landmark article Aug. 5, 1968: A Definition of Irreversible Coma. Report of the Ad Hoc Committee of the Harvard Medical School to Examine the Definition of Brain Death,” Journal of the American Medical Association 252(5): 677-679; Harvard Medical School 1968. “A Definition of Irreversible Coma. Report of the Ad Hoc Committee of the Harvard Medical School to Examine the Definition of Brain Death,” Journal of the American Medical Association 205(6): 337-340; Mollaret. P. and Goulon, M. 1959. “The Depassed Coma (Preliminary Memoir),” (in French, with English abstract at Pubmed), Revue Neurologique (Neurological Review, Paris, France) 101:3-15: Wertheimer, P., Jouvet, M., and Descotes, J. 1959. “Diagnosis of Death of the Nervous System in Comas with Respiratory Arrest Treated by Artificial Respiration,” (in French with English abstract at Pubmed), Presse Medicale (Medical Press, Paris, France) 67(3): 87-88).

Imaging methods are included here as allowing monitoring the status of patients along this path of descent from partial to complete failure (see, for example, Lehmenkühler, A. and Richter, F. 2020. “Cortical Spreading Depolarization (CSD) Recorded from Intact Skin, from Surface of Dura Mater or Cortex: Comparison with Intracortical Recordings in the Neocortex of Adult Rats,” Neurochemical Research 45(1): 34-41; An, J., Cho. E., Park, E., Lee, S. E., Han, M., Min, Y. G., and Chae, M. K. 2019. “Brain Computed Tomography Angiography in Postcardiac Arrest Patients and Neurologie Outcome,” Clinical and Experimental Emergency Medicine (Seoul, South Korea) 6(4): 297-302; Brown, L. S., Foster, C. G., Courtney, J. M., King. N. E., Howells, D. W., and Sutherland, B, A. 2019. “Pericytes and Neurovascular Function in the Healthy and Diseased Brain,” Online, Frontiers in Cellular Neuroscience 13:282; Caporarello, N., D'Angeli, F., Cambria, M. T., Candido, S., Giallongo, C., and 6 others 2019. “Pericytes in Microvessels: From “Mural” Function to Brain and Retina Regeneration,” Online, MDPI [Multidisciplinary Digital Publishing Institute] International Journal of Molecular Sciences (Basel, Switzerland) 20(24). pii: E6351; Erdener, S, E. and Dalkara, T. 2019. “Small Vessels are a Big Problem in Neurodegeneration and Neuroprotection,” Online, Frontiers in Neurology 10:889; Gastala, J., Fattal, D., Kirby, P. A., Capizzano, A. A., Sato, Y., and Moritani, T. 2019. “Brain Death: Radiologic Signs of a Non-radiologic Diagnosis,” Clinical Neurology and Neurosurgery (Amsterdam, North Holland, Netherlands) 185:105465; Lendahl. U., Nilsson, P., and Betsholtz, C. 2019. “Emerging Links between Cerebrovascular and Neurodegenerative Diseases-A Special Role for Pericytes,” European Molecular Biology Organization Reports 20(11): e48070; Pedicelli, A., Bartocci, M., Lozupone, E., D'Argento, F., Alexandre, A., and 5 others 2019. “The Role of Cervical Color Doppler Ultrasound in the Diagnosis of Brain Death,” Neuroradiology 61(2): 137-145; Rady, M. Y. 2019. “Neuroimaging of Intracranial Perfusion and the Clinical Diagnosis of Brain Death: Setting the Gold Standard in Humans,” Clinical Neuroradiology 29(3): 575-577; Santos, E., Olivares-Rivera, A., Major, S., Sánchez-Porras, R., Uhlmann, L., and 12 others 2019. “Lasting S-ketamine Block of Spreading Depolarizations in Subarachnoid Hemorrhage: A Retrospective Cohort Study,” Critical Care (London, England) 23(1): 427; Sawicki, M., Sołek-Pastuszka, J., Chamier-Ciemińska, K., Walecka, A., Walecki, J., and Bohatyrewicz, R. 2019. “Reply to Letter to the Editor “Neuroimaging of Intracranial Perfusion and the Clinical Diagnosis of Brain Death: Setting the Gold Standard in Humans,” Clinical Neuroradiology 29(3): 579-580; Yi, N. X., Zhou, L. Y., Wang, X. Y., Song. Y. J., Han, H. H., and 6 others 2019. “MK-801 [dizocilpine, an N-Methyl-D-aspartate antagonist] Attenuates Lesion Expansion following Acute Brain Injury in Rats: A Meta-analysis,” Neural Regeneration Research 14(11): 1919-1931; Ayata, C. 2018. “Monitoring Anoxic Depolarization at the Bedside: A Step Closer to the 24th Century,” Journal of Cerebral Blood Flow and Metabolism (Thousand Oaks, California) 38(7): 1123-1124; Dreier, J. P., Major, S., Foreman, B., Winkler, M. K. L., Kang, E. J., and 8 others 2018. “Terminal Spreading Depolarization and Electrical Silence in Death of Human Cerebral Cortex,” Annals of Neurology 83(2): 295-310; Foreman, B., Ngwenya, L. B., Stoddard, E., Hinzman, J. M., Andaluz, N., and Hartings, J, A. 2018. “Safety and Reliability of Bedside, Single Burr Hole Technique for Intracranial Multimodality Monitoring in Severe Traumatic Brain Injury,” Neurocritical Care 29(3): 469-480; Rizvi. T., Batchala, P., and Mukherjee, S. 2018. “Brain Death: Diagnosis and Imaging Techniques,” Seminars in Ultrasound, Computed Tomography, and Magnetic Resonance Imaging 39(5): 515-529; Shimizu, T., Hishikawa, T., Nishihiro, S., Shinji, Y., Takasugi, Y., and 9 others 2018. “NADH [nicotinamide adenine dinucleotide] Fluorescence Imaging and the Histological Impact of Cortical Spreading Depolarization during the Acute Phase of Subarachnoid Hemorrhage in Rats,” Journal of Neurosurgery 128(1): 137-143; Walter, U., Noachtar, S. and Hinrichs, H. 2018. “Digital Electroencephalography in Brain Death Diagnostics: Technical Requirements and Results of a Survey on the Compatibility with Medical Guidelines of Digital EEG Systems from Providers in Germany,” (in German with English abstract at Pubmed), Nervenarzt (Neurologist, Berlin, Germany) 89(2): 156-162; Dreier, J. P., Fabricius, M., Ayata, C., Sakowitz, O. W., Shuttleworth, C. W., and 85 others 2017. “Recording, Analysis, and Interpretation of Spreading Depolarizations in Neurointensive Care: Review and Recommendations of the COSBID [Co-Operative Studies on Brain Injury Depolarizations] Research Group,” Journal of Cerebral Blood Flow and Metabolism 37(5): 1595-1625; Hartings, J, A. 2017. “Spreading Depolarization Monitoring in Neurocritical Care of Acute Brain Injury,” Current Opinion in Critical Care 23(2): 94-102; Hartings, J. A., Shuttleworth, C. W., Kirov, S. A., Ayata, C., Hinzman, J. M., and 29 others 2017. “The Continuum of Spreading Depolarizations in Acute Cortical Lesion Development: Examining Leao's Legacy,” Journal of Cerebral Blood Flow and Metabolism 37(5): 1571-1594; Norton, L., Gibson, R. M., Gofton, T., Benson, C., Dhanani, S., and 4 others 2017. “Electroencephalographic Recordings during Withdrawal of Life-sustaining Therapy Until 30 Minutes after Declaration of Death,” Canadian Journal of Neurological Sciences 44(2): 139-145; O'Farrell, F. M., Mastitskaya, S., Hammond-Haley, M., Freitas, F., Wah, W, R. and Attwell, D. 2017. “Capillary Pericytes Mediate Coronary No-reflow after Myocardial Ischaemia,” e-life (Cambridge, England) 6. pii: e29280; Winkler, M. K., Dengler, N., Hecht, N., Hartings. J. A., Kang, E. J., and 5 others 2017. “Oxygen Availability and Spreading Depolarizations Provide Complementary Prognostic Information in Neuromonitoring of Aneurysmal Subarachnoid Hemorrhage Patients,” Journal of Cerebral Blood Flow and Metabolism 37(5): 1841-1856; Pana, R., Hornby, L., Shemie, S. D., Dhanani, S., and Teitelbaum, J. 2016. “Time to Loss of Brain Function and Activity during Circulatory Arrest,” Journal of Critical Care 34:77-83; Rady, M. Y. and Verheijde, J. L. 2016. “Neuroscience and Awareness in the Dying Human Brain: Implications for Organ Donation Practices,” Journal of Critical Care 34:121-123; Dalkara, T. and Alarcon-Martinez, L. 2015. “Cerebral Microvascular Pericytes and Neurogliovascular Signaling in Health and Disease,” Brain Research 1623:3-17; Kaindl, A. M., Degos, V., Peineau, S., Gouadon, E., Chhor, V., and 14 others 2012. “Activation of Microglial N-methyl-D-aspartate Receptors Triggers Inflammation and Neuronal Cell Death in the Developing and Mature Brain,” Annals of Neurology 72(4): 536-549; Yemisci, M., Gursoy-Ozdemir, Y., Vural, A., Can, A., Topalkara, K., and Dalkara, T. 2009. “Pericyte Contraction Induced by Oxidative-nitrative Stress Impairs Capillary Reflow Despite Successful Opening of an Occluded Cerebral Artery,” Nature Medicine 15(9): 1031-1037).

Until direct stimulation thus becomes a practical reality, mechanical ventilation, which likewise affords constancy in rate and tidal volume, should be used. Since other brain functions will have been extinguished, or effectively denervated, perceptual and emotional factors which normally adjust breathing in response to exigencies of the moment will have been eliminated. With no exertion, emotion, or seizures, for example, spontaneous respiration should proceed at a constant rate and depth, eliminating the variability that would normally arise in response to contextual factors to reduce constancy as a controlled variable.

Given that collateral cerebral pathology, such as epilepsy, and breathing problems of pulmonary rather than cerebral origin can provoke cardiac dysrhythmias, the prevention of irregularities in breathing reduces the potential complications to be encountered in single and double switch heart transplantation, for example (see, for example, Ben-Tal, A., Wang. Y., and Leite, M. C. A. 2019. “The Logic behind Neural Control of Breathing Pattern,” (Nature Research) Scientific Reports 9(1): 9078; Buchner, T. 2019. “A Quantitative Model of Relation between Respiratory-related Blood Pressure Fluctuations and the Respiratory Sinus Arrhythmia,” Medical and Biological Engineering and Computing 57(5): 1069-1078; Liu, H., Yang, Z., Meng. F., Guan, Y., Ma. Y., and 8 others 2019. “Deceleration and Acceleration Capacities of Heart Rate in Patients with Drug-resistant Epilepsy,” Clinical Autonomic Research 29(2): 195-204; Noble, D. J. and Hochman, S. 2019. “Hypothesis: Pulmonary Afferent Activity Patterns during Slow, Deep Breathing Contribute to the Neural Induction of Physiological Relaxation,” Online, Frontiers in Physiology 10:1176; Soos, M. P. and McComb, D. 2019. “Sinus Arrhythmia,” Treasure Island, Florida: StatPearls Publishing Co., Online at https://www.ncbi.nlm.nih.gov/books/NBK537011/; Buzea, C. A., Dan, G. A., Dan, A. R., Delcea. C., Balea, M. I., and 3 others 2017. “Deceleration and Acceleration Capacities in Risk Stratification for Arrhythmias in Patients With Chronic Obstructive Pulmonary Disease,” American Journal of Therapeutics 24(1): e44-e51; Karemaker, J. M. and DeBoer, R. W. 2017. “Vagal Baroreflex Latency in Circulatory Control,” Journal of Physiology 595(6): 2197-2198; Rusinowicz, T., Zielonka, TM2, and Zycinska, K. 2017. “Cardiac Arrhythmias in Patients with Exacerbation of COPD,” Advances in Experimental Medicine and Biology 1022: 53-62; Yeh, J. J., Wei. Y. F., Lin, C, L., an Hsu. W, H. 2017. “Association of Asthma-chronic Obstructive Pulmonary Disease Overlap Syndrome with Coronary Artery Disease, Cardiac Dysrhythmia and Heart Failure: A Population-based Retrospective Cohort Study,” Online, British Medical Journal Open 7(10): e017657; Eckberg, D, L., Cooke, W. H., Diedrich, A., Biaggioni, I., Buckey, J, C. Jr., and 12 others 2016. “Respiratory Modulation of Human Autonomic Function on Earth,” Journal of Physiology 594(19): 5611-5627; Ben-Tal. A., Shamailov, S. S., and Paton, J, F. 2014. “Central Regulation of Heart Rate and the Appearance of Respiratory Sinus Arrhythmia: New Insights from Mathematical Modeling,” Mathematical Biosciences 255:71-82; Sin, P. Y., Webber, M. R., Galletly, D. C., Ainslie, P. N., Brown, S., and 4 others 2010. “Interactions between Heart Rate Variability and Pulmonary Gas Exchange Efficiency in Humans,” Experimental Physiology 95(7): 788-797; Grossman, P. and Taylor, E. W. 2007. “Toward Understanding Respiratory Sinus Arrhythmia: Relations to Cardiac Vagal Tone, Evolution, and Biobehavioral Functions,” Biological Psychology 74(2): 263-285; Yasuma, F. and Hayano. J. 2004. “Respiratory Sinus Arrhythmia: Why Does the Heartbeat Synchronize with Respiratory Rhythm?,” Chest 125(2): 683-690; Taylor, J. A., Myers, C. W., Halliwill, J. R., Seidel, H., and Eckberg, D. L. 2001. “Sympathetic Restraint of Respiratory Sinus Arrhythmia: Implications for Vagal-Cardiac Tone Assessment in Humans,” American Journal of Physiology. Heart and Circulatory Physiology 280(6): H2804-H2814; Hirsch, J, A. and Bishop, B. 1981. “Respiratory Sinus Arrhythmia in Humans: How Breathing Pattern Modulates Heart Rate,” American Journal of Physiology 241(4): H620-H629; British Medical Journal 1969. “Respiration and Heart Rhythm,” Online, British Medical Journal 4(5682): 514-515).

Pertinent to the sustainment of a fetal donor past death, literature concerning the anatomy and physiology of the brainstem breathing and circulatory control centers in the fetus is included among the references cited. Apart from avoiding the potential injury that can result from the applications of electrical shocks to the heart, light targeting is far more tightly focused. Neuromodulation through electrostimulation and prospectively, optogenetics, is addressed here with respect to sustaining a deceased donor beyond the term afforded by extracorporeal membrane oxygenation, hence, primarily with respect first, to respiration and as addressed below, circulation.

In this section, optogenetics is addressed not only as a means for direct stimulation of the respiratory and circulatory centers in the brainstem, but as an adjunct to reduce the intensity if not an alternative to avoid entirely the potential injury of electrical shocks released by cardioverter defibrillators, for example, as pertinent to heart transplantation. Along with electrical and chemical stimulation, by direct stimulation of the pneumotaxic and apneustic centers of the pontine respiratory group and the dorsal and ventral respiratory groups in the medulla, optogenetics also has potential application to the sustainment of spontaneous ventilation in a brain-dead organ donor. Analogous measures may allow the preservation past death of body temperature.

Sustainment thus without mechanical manipulation should sustain ‘normal’ breathing, or spontaneous ventilation, longer and without the complications of mechanically assisted ventilation (see, for example, Menuet, C., Connelly, A. A., Bassi, J. K., Melo, M. R., Le, S., and 5 others 2020. “PreBotzinger Complex Neurons Drive Respiratory Modulation of Blood Pressure and Heart Rate,” Online, Elife (Cambridge, England) 9: e57288; Giardini, F., Biasci, V., Scardigli, M., Pavone, F. S., Bub, G., and Sacconi, L. 2019. “A Software Architecture to Mimic a Ventricular Tachycardia in Intact Murine Hearts by Means of an All-optical Platform,” Methods and Protocols (Basel, Switzerland) 2(1). pii: E7; Mickle, A. D., Won, S. M., Noh, K. N., Yoon, J., Meacham, K. W., and 20 others 2019. “A Wireless Closed-loop System for Optogenetic Peripheral Neuromodulation,” Nature 565(7739): 361-365; Sasse, P., Funken, M., Beiert, T., and Bruegmann, T. 2019. “Optogenetic Termination of Cardiac Arrhythmia: Mechanistic Enlightenment and Therapeutic Application?,” Online, Frontiers in Physiology 10:675; Zaglia, T., Di Bona, A., and Mongillo, M. 2019. “A Light Wand to Untangle the Myocardial Cell Network,” Methods and Protocols (Basel, Switzerland) 2(2). pii: E34; Boyle, P. M., Karathanos, T. V., and Trayanova, N, A. 2018. “Cardiac Optogenetics: 2018,” Journal of the American College of Cardiology. Clinical Electrophysiology 4(2): 155-167; Boyle, P. M., Murphy, M. J., Karathanos, T. V., Zahid, S., Blake, R, C. 3rd, and Trayanova, N. A. 2018. “Termination of Re-entrant Atrial Tachycardia via Optogenetic Stimulation with Optimized Spatial Targeting: Insights from Computational Models,” Journal of Physiology 596(2): 181-196; Malheiros-Lima, M. R., Totola, L. T., Lana, M. V. G., Strauss, B. E., Takakura, A. C., and Moreira, T. S. 2018. “Breathing Responses Produced by Optogenetic Stimulation of Adrenergic Cl Neurons are Dependent on the Connection with preBotzinger Complex in Rats,” Pflügers Archiv: European Journal of Physiology 470(11): 1659-1672; Scardigli, M., Mullenbroich, C., Margoni, E., Cannazzaro, S., Crocini, C., and 12 others 2018. “Real-time Optical Manipulation of Cardiac Conduction in Intact Hearts,” Journal of Physiology 596(17): 3841-3858; Crocini, C., Ferrantini, C., Pavone, F. S., and Sacconi, L. 2017. “Optogenetics Gets to the Heart: A Guiding Light beyond Defibrillation,” Progress in Biophysics and Molecular Biology 130 (Part B): 132-139; Zhao, H. 2017. “Recent Progress of Development of Optogenetic Implantable Neural Probes,” Online, MDPI [Multidisciplinary Digital Publishing Institute] International Journal of Molecular Sciences (Basel, Switzerland) 18(8). pii: E1751; Bruegmann, T., Boyle, P. M., Vogt, C. C., Karathanos, T. V., Arevalo, H. J., and 3 others 2016. “Optogenetic Defibrillation Terminates Ventricular Arrhythmia in Mouse Hearts and Human Simulations,” Journal of Clinical Investigation 126(10): 3894-3904; Karathanos, T. V., Bayer, J. D., Wang, D., Boyle, P. M., and Trayanova, N, A. 2016. “Opsin Spectral Sensitivity Determines the Effectiveness of Optogenetic Termination of Ventricular Fibrillation in the Human Heart: A Simulation Study,” Journal of Physiology 594(23): 6879-6891; Steude, A., Witts, E. C., Miles, G. B., and Gather, M, C. 2016. “Arrays of Microscopic Organic LEDs for High-resolution Optogenetics,” Science Advances 2(5): e1600061; Alsahafi, Z., Dickson, C. T., and Pagliardini, S. 2015. “Optogenetic Excitation of preBotzinger Complex Neurons Potently Drives Inspiratory Activity in Vivo,” Journal of Physiology 593(16): 3673-3692; Fan, B. and Li, W. 2015. “Miniaturized Optogenetic Neural Implants: A Review,” Lab on a Chip 15(19): 3838-3855; Sherman, D., Worrell. J. W., Cui. Y., and Feldman, J. L. 2015. “Optogenetic Perturbation of PreBötzinger Complex Inhibitory Neurons Modulates Respiratory Pattern,” Nature Neuroscience 18(3): 408-414; Pashaie. R., Anikeeva, P., Lee, J. H., Prakash. R., Yizhar, O., and 4 others 2014. “Optogenetic Brain Interfaces,” Institute of Electrical and Electronics Engineers Reviews in Biomedical Engineering 7:3-30).

Optogenetics also has potential applications to the urinary tract, both related and unrelated to kidney transplantation, for example (see, for example, Kessler, T. M., Birder, L. A., and Gomery, P. 2019. “Neuromoduation of Urinary Tract Function,” New England Journal of Medicine 380(21): 2067-2069; Park, E. and Lee, K. S. 2019. “A New Approach to Urinary Bladder Control with Optogenetics,” Investigative and Clinical Urology 60(2): 61-63; Roche, E. T. 2019. “Implanted Device Enables Responsive Bladder Control,” Nature 565(7739): 298-300; Roy, H, A. and Green, A. L. 2019. “The Central Autonomie Network and Regulation of Bladder Function,” Online, Frontiers in Neuroscience 13:535; DeBerry, J. J., Samineni. V. K., Copits, B. A., Sullivan, C. J., Vogt. S. K., and 3 others 2018. “Differential Regulation of Bladder Pain and Voiding Function by Sensory Afferent Populations Revealed by Selective Optogenetic Activation,” Online, Frontiers in Integrative Neuroscience 12:5; Kessler. T. M., Burkhard, F. C., Z'Brun, S., Stibal, A., Studer, U. E., Hess, C. W., and Kaelin-Lang, A. 2008. “Effect of Thalamic Deep Brain Stimulation on Lower Urinary Tract Function,” European Urology 53(3): 607-612).

Innate control over cardiovascular function is largely elucidated (see, for example, Chaudhry, R. and Rehman, A. 2019. “Physiology, Cardiovascular,” Treasure Island, Florida: StatPearls Publishing Co. Online at https://www.ncbi.nlm.nih.gov/books/NBK493197/; Bioengineering and Physiology Faculty, University of Utah 2019. Control of Circulation Physiologie Control Systems, slide presentation Online at http://www.sci.utah.edu/˜macleod/bioen/be6000/notes/L12-control-circ.pdf; McAllen, R. M. and Mckinley, M. J. 2018. “Efferent Thermoregulatory Pathways Regulating Cutaneous Blood Flow and Sweating,” Handbook of Clinical Neurology 156:305-316; Joyner, M. J., Barnes, J. N., Hart, E. C., Wallin, B. G., and Charkoudian, N. 2015. “Neural Control of the Circulation: How Sex and Age Differences Interact in Humans,” Comprehensive Physiology 5(1): 193-215; Michelini, L. C., O'Leary, D. S., Raven, P. B., and Nobrega, A, C. 2015. “Neural Control of Circulation and Exercise: A Translational Approach Disclosing Interactions between Central Command, Arterial Baroreflex, and Muscle Metaboreflex,” American Journal of Physiology. Heart and Circulatory Physiology 309(3): H381-H392; Thomas, G, D. 2011. “Neural Control of the Circulation,” Advances in Physiology Education 35(1): 28-32; Pittman, R. N. 2011. “Regulation of Tissue Oxygenation,” Chapter 2. The Circulatory System and Oxygen Transport, San Rafael, California: Morgan and Claypool Life Sciences; Van de Louw, A., Medigue, C., Papelier, Y., Landrain, M., and Cottin, F. 2010. Op cit. this section; Nicholls, J, G. and Paton, J, F. 2009. “Brainstem: Neural Networks Vital for Life,” Philosophical Transactions of the Royal Society of London. Series B. Biological Sciences 364(1529): 2447-2451; Maeda, M., Inoue, M., Takao, S., and Nakai, M. 1999. “Central Control Mechanisms of Circulation in the Medulla Oblongata by Nitric Oxide,” Japanese Journal of Physiology 49(6): 467-478; Hurnes, M. (ed.) 1980. Neural Control of Circulation, Cambridge, Massachusetts: Academic Press/Elsevier; McDowall, R. J. S., Malcomson, G. E., McWhan, I. 1939. The Control of the Circulation of the Blood, New York, New York: Longmans Green and Company).

Currently, ambulatory outpatients with end-stage disease at risk of death can have an artificial organ and sensor-actuated switch implanted to sustain function pending transfer to a medical center which may already have, or if not, can call for a donor organ. However, rather than to have continued intensive somatic support of the body past death, life support will have been shut down, the body moved to the morgue, where circulation will continue for days but will be unstable and eventually stop.

The condition of a chronic, persistent, or permanent vegetative state is encountered infrequently enough as to warrant no more than brief comment. Patients in a persistent vegetative unresponsive wakefulness state will seldom—in one study, some 18 percent—feel pain (Calabro, R. S., Pignolo, L., Muller-Eising. C., and Naro, A. 2021. “Pain Perception in Disorder of Consciousness. A Scoping Review on Current Knowledge, Clinical Applications, and Future Perspective,” Online, Brain Sciences 11(5): 665; Calabro, R. S., Naro, A., Manuli. A., Leo, A., De Luca, R., and 4 others 2017. “Pain Perception in Patients with Chronic Disorders of Consciousness. What Can Limbic System Tell Us?,” Clinical Neurophysiology 128(3): 454-462; Naro, A. Leo, A. Cannavo, A., Buda, A., Bramanti, P., and Calabro, R. S. 2016. “Do Unresponsive Wakefulness Syndrome Patients Feel Pain? Role of Laser-evoked Potential-induced Gamma-band Oscillations in Detecting Cortical Pain Processing,” Neuroscience 317:141-148; Pistoia, F., Sacco, S., Stewart, J., Sara, M., and Carolei, A. 2016. “Disorders of Consciousness: Painless or Painful Conditions? Evidence from Neuroimaging Studies,” Online, Brain Sciences 6(4): 47; Markl. A., Yu, T., Vogel, D., Muller, F., Kotchoubey, B., and Lang, S. 2013. “Brain Processing of Pain in Patients with Unresponsive Wakefulness Syndrome,” Online, Brain and Behavior 3(2): 95-103). Some do is not an answer, but either way, those long maintained on life support have organs not likely to be acceptable for transplantation anyway.

It should be considered that to sustain a corpse with no autonomie function compelled toward autolysis and decomposition in an organ donor condition is fundamentally more complex than is sustaining a patient in a persistent vegetative state, without higher brain function but retaining a functioning brainstem and autonomie function (Beshish, A. G., Bradley, J. D., McDonough, K, L., Halligan, N. L. N., McHugh, W. M. and 4 others 2019. “The Functional Immune Response of Patients on Extracorporeal Life Support,” American Society for Artificial Internal Organs Journal 65(1): 77-83; Shetty, V, L., Mali, S. S., Shetty, S. V., and Shinde, P, D. 2017. Op cit.). Allowing the patient to pass into a condition of ‘brain death’ only makes the organs subject to the integrated stress response, the ‘cytokine-storm’ generating havoc associated with death, and a critically injurious period of anoxia, making the intentional cessation of perfusion precisely wrong in causing the needless degradation of the organs.

The small vessels of the graft organ then become more permeable, releasing fluid into the tissue, and the oxidative stress of oxygen deprivation results in chemical damage to the cell contents, so that following even a brief episode of ischemia, the restoration of blood flow draws leukocytes that release inflammatory factors causing further injury to the graft organ which can result in rejection. Still missing will have been a means for accomplishing transfer of the graft organ from the dead donor or if unavoidable, perfusion machine, to the recipient with no interruption in perfusion, no hypoxia in the deceased donor, and no ischemia of the graft organ before or during harvesting.

Patients who cannot be conclusively determined to be incapable of recovering consciousness are prohibited from organ harvesting, and to have remained in an unconscious state for a long time suggests that their organs may no longer be suitable for transplantation. Provided the organs are judged adequate for transplantation, a patient who appears effectively dead, meets the criteria of lacking pain sensation, and who has not been on life support for more than a half year or so should be retained on ECMO during organ harvesting.

The question of pain perception remains inconclusive, but it would appear that most such patients do not feel pain (see, for example; Calabro, R. S., Pignolo, L., Müller-Eising. C., and Naro, A. 2021. “Pain Perception in Disorder of Consciousness: A Scoping Review on Current Knowledge, Clinical Applications, and Future Perspective,” Online, MDPI [Multidisciplinary Digital Publishing Institute] Brain Sciences (Basel, Switzerland) 11(5): 665; Riganello, F. Vatrano, M., Carozzo, S., Russo, M., Lucca, L. F., and 4 others 2021. “The Timecourse of Electrophysiological Brain-Heart Interaction in DoC [disorders of consciousness] Patients,” Online, MDPI [Multidisciplinary Digital Publishing Institute] Brain Sciences (Basel, Switzerland) 11(6): 750; Riganello, F. Soddu, A., and Tonin, P. 2021. “Addressing Pain for a Proper Rehabilitation Process in Patients with Severe Disorders of Consciousness,” Online, Frontiers in Pharmacology 12:628980; Riganello, F., Larroque, S. K., Di Perri, C., Prada, V., Sannita, W. G., and Laureys, S. 2019. “Measures of CNS-Autonomic Interaction and Responsiveness in Disorder of Consciousness,” Online, Frontiers in Neuroscience 13:530; Riganello, F., Larroque, S. K., Bahri, M. A., Heine, L., Martial, and 7 others 2018. “A Heartbeat Away from Consciousness: Heart Rate Variability Entropy Can Discriminate Disorders of Consciousness and is Correlated with Resting-state fMRI Brain Connectivity of the Central Autonomic Network,” Online, Frontiers in Neurology 9:769; Schnakers, C., Chatelle, C., Demertzi. A., Majerus, S. and Laureys, S. 2012. “What about Pain in Disorders of Consciousness?,” Online, American Association of Pharmaceutical Scientists Journal 14(3): 437-444; Riganello, F., Maori, S., Alleva, E., Petrini, C., Soddu, A., Leon-Carrion, J., and Dolce, G. 2016. “Pain Perception in Unresponsive Wakefulness Syndrome May Challenge the Interruption of Artificial Nutrition and Hydration: Neuroethics in Action,” Online, Frontiers in Neurology 7:202; Demertzi. A., Schnakers, C., Ledoux, D., Chatelle. C., Cruno. C., and 3 others 2009. “Different Beliefs about Pain Perception in the Vegetative and Minimally Conscious States: A European Survey of Medical and Paramedical Professionals,” Progress in Brain Research 177:329-338).

Maintaining cardiorespiratory function upon death is essential to preserve the organs in a living condition (Fuchs, M., Schibilsky, D., Zeh, W., Berchtold-Herz, M., Beyersdorf, F., and Siepe, M. 2019. “Does the Heart Transplant have a Future?,” European Journal of Cardiothoracic Surgery 55 (Supplement 1): 138-148; Machado, C. 2010. “Diagnosis of Brain Death,” Neurology International 2(1): e2; Rosenberg, R. N. 2009. “Consciousness, Coma, and Brain Death-2009,” Journal of the American Medical Association 301(11): 1172-1174; Rolston, H. 3rd. 1982. “The Irreversibly Comatose: Respect for the Subhuman in Human Life,” Journal of Medicine and Philosophy 7(4): 337-354; Ad Hoc Committee of the Harvard Medical School 1968. “A Definition of Irreversible Coma. Report of the Ad Hoc Committee of the Harvard Medical School to Examine the Definition of Brain Death,” Journal of the American Medical Association 205(6): 337-340).

By making possible sudden or metered switching of the recipient's circulation from his own end-stage solid organ to that of a donor, intravascular switching valves and servovalves allow transplantation from the donor without interruption in circulation. There is, therefore, zero ischemia time, and with a heart, zero impairment of the cardionector (cardionecteur, conduction system of the heart), eliminating the risk of heart block and arrhythmia, making possible off-pump, or beating heart, and cross-clamp, chilling, and cardioplegic solution-free heart transplantation. In metered compound vascular bypass, or sudden switched-organ transplantation, blood flow through the graft organ is continuously and gradually replaced with the blood of the recipient, thus transferring the organ from the circulatory system of the donor to that of the recipient.

Along with the direct pipeline targeting to the transplant organ of supportive agents to include immunosuppressants, anti-inflammatoires, and antimicrobials, for example, the endothelia throughout the graft organ are washed over with the blood of the recipient. The release of these agents is executed by an implanted automatic disorder response system in response to data sent to its control processor by implanted sensors. The white blood cells of the donor substantially removed along the endothelia, the risk of graft versus host and host versus graft rejection is reduced and some measure of microchimerization accomplished (references below in this section), reducing the immune response of abrupt contact with alien tissue.

Moreover, the fact that using the metered switch method the endothelium is exposed to the blood of the donor likely accomplishes a measure of immune tolerance induction that also pre-attenuates the sequelae due to hypercholesterolemia, then vasculopathy, which ensues following a heart transplant, more likely in a replacement heart with atherosed coronaries, for example, and a previously implanted automatic disorder response system as described in U.S. Pat. No. 11,759,186, entitled Ductus Side-entry Jackets and Prosthetic Disorder Response Systems used to accomplish the transplantation can also target medication such as evolocumab (Repatha®, Amgen, Newbury Park/Thousand Oaks, California) and alirocumab (Praluente, Regeneron Pharmaceuticals Incorporated, Eastview, New York) and/or a statin directly to the heart following transplantation to further reduce the risk of hypercholesterolemia and vasculopathy (see, for example, Küh1, M., Binner, C., Jozwiak, J., Fischer, J., Hahn, J., and 5 others 2019. “Treatment of Hypercholesterolaemia with PCSK9 [proprotein convertase subtilisin-kexin type 9] Inhibitors in Patients after Cardiac Transplantation,” Online, PLoS [Public Library of Science] One 14(1): e0210373; Alkhalil, M. 2019. “Proprotein Convertase Subtilisin/Kexin Type 9 (PCSK9) Inhibitors, Reality or Dream in Managing Patients with Cardiovascular Disease,” Current Drug Metabolism 20(1): 72-82; Auer, J. and Berent, R. 2018. “Alirocumab as Add-on Therapy to Statins: Current Evidence and Clinical Potential,” Therapeutic Advances in Cardiovascular Disease 12(7): 191-202; Handelsman, Y. and Lepor, N, E. 2018. “PCSK9 Inhibitors in Lipid Management of Patients with Diabetes Mellitus and High Cardiovascular Risk: A Review,” Journal of the American Heart Association (Oxford, England) 7(13). pii: e008953; Nohara, A., Ohmura, H., Okazaki, H., Ogura, M., Kitagawa, K., and 4 others 2018. “Statement for Appropriate Clinical Use of PCSK9 Inhibitors,” Journal of Atherosclerosis and Thrombosis 25(8): 747-750; Ogura, M. 2018. “PCSK9 Inhibition in the Management of Familial Hypercholesterolemia,” Journal of Cardiology 71(1): 1-7; Pokrywka, G. S. 2018. “PCSK9 Inhibitors: A Non-statin Cholesterol-lowering Treatment Option,” Postgraduate Medicine 130(3): 287-298; Pradhan, A. D., Aday, A. W., Rose, L. M., and Ridker, P. M. 2018. “Residual Inflammatory Risk on Treatment with PCSK9 Inhibition and Statin Therapy,” Circulation 138(2): 141-149; Ridker, P. M., Rose, L. M., Kastelein, J. J. P., Santos, R. D., Wei, C., and 4 others 2018. “Cardiovascular Event Reduction with PCSK9 Inhibition among 1578 Patients with Familial Hypercholesterolemia: Results from the SPIRE [Studies of PCSK9 Inhibition and the Reduction of vascular Events] Randomized Trials of Bococizumab,” Journal of Clinical Lipidology 12(4): 958-965; Zodda, D., Giammona, R., and Schifilliti, S. 2018. “Treatment Strategy for Dyslipidemia in Cardiovascular Disease Prevention: Focus on Old and New Drugs,” Pharmacy (Basel, Switzerland) 6(1). pii: E10; Chaudhary, R., Garg, J., Shah, N., and Sumner, A. 2017. “PCSK9 Inhibitors: A New Era of Lipid Lowering Therapy,” World Journal of Cardiology 9(2): 76-91; Descamps, O. S., Fraass, U., Dent, R., Marz, W., and Gouni-Berthold, I. 2017. “Anti-PCSK9 Antibodies for Hypercholesterolaemia: Overview of Clinical Data and Implications for Primary Care,” International Journal of Clinical Practice 71(8); Nozue, T. 2017. “Lipid Lowering Therapy and Circulating PCSK9 Concentration,” Journal of Atherosclerosis and Thrombosis 24(9): 895-907; Agabiti Rosei, E. and Salvetti, M. 2016. “Management of Hypercholesterolemia, Appropriateness of Therapeutic Approaches and New Drugs in Patients with High Cardiovascular Risk,” High Blood Pressure and Cardiovascular Prevention 23(3): 217-230).

Monoclonal antibody, PCSK9 inhibitors such as alirocumab (Praluente, Regeneron Pharmaceuticals Incorporated, Eastview, New York) call for injection every two to four weeks. A more recently FDA approved signal interference-based LDL reduction drug, inclisiran (Leqvio®, Novartis) requires injection no more than twice yearly, and bempedoic acid (Nexletol®, Esverion®, Esperion Therapeutics) calls for taking one tablet daily, doing away with the need for injection. These drugs are often compounded with a statin or ezetimibe, which latter has yet another action-decreasing the absorption of cholesterol in the small intestine, and thus forcing its acquisition from the circulation.

Drugs are enumerated to allow emphasis that an automatic prosthetic disorder response system can deliver any drug however formulated into solid or fluid tissue or any ductus anywhere in the body and can do so using any appropriate end-effector, and can do so whether by subcutaneous or solid tissue injection, infusion directly into the pulmonary or systemic arterial or venous trees, direct release of the drug in water solution into the digestive tract, or by affecting solid or fluid tissue through irradiation, only topical administration at the body surface or insertion of a rectal suppository appropriately accepted.

Provided the system has been properly planned on the basis of the medical history, thorough physical examination, and genetic assessment of the individual patient and an evaluation as to the plausible sequelae that could follow the initial treatment, the system can be designed ab initio to target the proper support at the affected site, reducing the potential reasons for reentry for revision or supplementation. Halfway measures that defer all else to the avoidance of a relatively safe initially decisive and durable invasive procedure may buy short term comfort at the expense of more serious treatment. Equally if not more significantly, the system automatically discharges its prescription-program according to the schedule and dosing specified for a patient who requires such support.

Furthermore, in a patient who needs many drugs, the system will continuously monitor the effect of and adjust the dosing of each to achieve the optimum overall effect across the combination. An end result is that through continuous surveillance, the system reduces postoperative risk. In so doing, it makes routinely disqualified prospects and organs qualify for transplantation, materially increasing the pool of transplant organs.

Both sudden and metered compound vascular bypass, or sudden switched-organ transplantation eliminate:

1. Ischemia and reperfusion injury.

2. The need to place the recipient on cardiopulmonary bypass or extracorporeal membrane oxygenation with aortic cross-clamping, carrying risk of complications.

3. The need for general rather than regional anesthesia with the risk of postperfusion cognitive deficits and decline in ventricular perfusion and function following heart-lung machine-supported heart transplantation.

4. The risk of problem bleeding, especially in liver transplantation.

5. Incision into the graft organ, and therewith, the risk of surgical trauma which can result in adhesions, scar tissue, and postoperative dysrhythmia that would interfere with or jeopardize a later retransplantation or surgical repair of the transplant organ or its blood supply, such as a later need for coronary artery bypass grafting or valve replacement, and

6. The side-entry jackets with accessory channels (sidelines; druglines) if not the mainlines (bloodlines) left in place once the recipient is closed, a means for directly piping medication to the blood supply of the graft organ not only allows the immediately responsive, automatic direct targeting of immunosuppressants to the site but by allowing similar delivery of pathophysiologically counteracting substances, expands the zone of extended criteria donor (less than ideal, marginal) organs for transplantation, to some extent alleviating the severe shortage of organs available for transplantation.

7. In switch transplantation, the flow of blood through the donor organ and its supply and drainage vessels in the recipient is uninterrupted, and an anticoagulant or thrombolytic can be directly targeted to these, so that thrombosis is eliminated, making liver transplantation in particular less susceptible to complications. 8. By allowing excessive pressure to be diverted to nearby large vessels, diversion valves can be used to avoid ‘big-heart.’ or hyperperfusion syndrome where the output of a new heart larger than would spontaneously self-adjust to the smaller load or one not so large but posing a danger during the days preceding self-adjustment could then be used as a bridge or for the life of the implant (Shin, H. J., Jhang, W. K., Park, J. J., Yun, T. J., Kim, Y. H., and 3 others 2011. Op cit., this section), making an oversized graft organ usable, the shortage in infant hearts otherwise severe.

9. Metered switch technique allows immune tolerance induction concurrent with transfer of the graft organ from the circulatory system of the donor into that of the recipient.

By making possible continuity of perfusion during switch transplantation, minimizing surgical trauma in organ transplantation, ductus side-entry diversion valve-jackets and servovalve-jackets offer critical advantages that improve the odds for transplant success. In eliminating incision into both donor and recipient hearts, the extracardiac compound vascular bypass heart transplantation made possible by vascular valves or servovalves reduces the surgical trauma of heart transplantation to the extent that less robust hearts can be used, expanding the zone and therefor the pool of available hearts.

Also made possible by vascular valves, where the ischemia and hypoperfusion-free cross-shunt method described in section 2b(2)(b), entitled Bidirectional Valves for Perinatal Extracardiac Correction of Complete, or Dextro-transposition of the Great Arteries and Reciprocal Cross-circulation, for the extracardiac correction of a total or dextro-transposition of the great arteries can be applied, the complete switching of blood flow through the aorta and pulmonary artery accomplished without incision into the heart or great vessels, much reducing recovery time and increasing the odds for a favorable outcome.

Pending the development of permanently and dependably anticoagulant, or antithrombogenic, and antibiofouling, antibiofilm-coated or impregnated tubing, a slow drip of heparin or another anticoagulant transmitted through the accessory channels will serve to dispel the clogging of the blood conveying lines, or mainlines, by clot (see, for example, Liu, Z., Fang, L., Delaittre, G., Ke, Y., and Wu, G. 2019. “Heparinized Polyurethane Surface via a One-step Photografting Method,” Online, MDPI [Multidisciplinary Digital Publishing Institute] Molecules (Basel, Switzerland) 24(4). pii: E758; Howell, C., Grinthal, A., Sunny, S., Aizenberg, M., and Aizenberg, J. 2018. “Designing Liquid-infused Surfaces for Medical Applications: A Review,” Advanced Materials (Deerfield Beach, Florida) 30(50): e1802724; Liu, F., Hansra, S., Crockford, G., Köster, W., Allan, B. J., and 3 others 2018. “Tetrasodium EDTA [ethylenediaminetetraacetic acid] is Effective at Eradicating Biofilms Formed by Clinically Relevant Microorganisms from Patients' Central Venous Catheters,” Online, mSphere (Washington, D.C.) 3(6). pii: e00525-18; Nahain, A. A., Ignjatovic, V., Monagle, P., Tsanaktsidis, J., and Ferro, V. 2018. “Heparin Mimetics with Anticoagulant Activity,” Medicinal Research Reviews 38(5): 1582-1613; Mendes Marques, M, L., Presa, F. B., Viana, R. L. S., Costa, M. S. S. P., Amorim, M. O. R., and 5 others 2018. “Anti-thrombin, Anti-adhesive, Anti-migratory, and Anti-proliferative Activities of Sulfated Galactans from the Tropical Green Seaweed, Udotea flabellum,” Marine Drugs 17(1). pii: E5; He, W., Liu, P., Zhang, J., and Yao, X. 2018. “Emerging Applications of Bioinspired Slippery Surfaces in Biomedical Fields” Chemistry (Weinheim an der Bergstrasse, Germany) 24(56): 14864-14877; Pathak, R., Bierman, S. F., and d'Arnaud, P. 2018. “Inhibition of Bacterial Attachment and Biofilm Formation by a Novel Intravenous Catheter Material Using an in Vitro Percutaneous Catheter Insertion Model,” Medical Devices (Auckland, New Zealand) 11:427-432; Sotiri, I., Tajik, A., Lai. Y., Zhang, C. T., Kovalenko. Y., and 9 others 2018. “Tunability of Liquid-infused Silicone Materials for Biointerfaces,” Biointerphases 13(6): 06D401; Gominet, M., Compain, F., Beloin, C., and Lebeaux, D. 2017. “Central Venous Catheters and Biofilms: Where Do We Stand in 2017?,” Acta Pathologica, Microbiologica, et Immunologica Scandinavica 125(4): 365-375; Kovalenko, Y., Sotiri, I., Timonen, J. V. I., Overton, J. C., Holmes, G., Aizenberg, J., and Howell, C. 2017. “Bacterial Interactions with Immobilized Liquid Layers,” Advanced Healthcare Materials 6(15); Wallace, A., Albadawi, H., Patel, N., Khademhosseini, A., Zhang, Y. S., and 3 others 2017. “Anti-fouling Strategies for Central Venous Catheters,” Cardiovascular Diagnosis and Therapy 7 (Supplement 3): S246-S257; Zhang, P., Lin, L., Zang. D., Guo, X., and Liu, M. 2017. “Designing Bioinspired Anti-biofouling Surfaces Based on a Superwettability Strategy,” Small (Weinheim an der Bergstrasse, Germany) 13(4); Zia, F., Zia, K. M., Zuber, M., Tabasum, S., and Rehman, S. 2016. “Heparin Based Polyurethanes: A State-of-the-art Review,” International Journal of Biological Macromolecules 84:101-111; Sotiri, I., Overton, J. C., Waterhouse, A., and Howell, C. 2016. “Immobilized Liquid Layers: A New Approach to Anti-adhesion Surfaces for Medical Applications,” Experimental Biology and Medicine (Maywood, New Jersey) 241(9): 909-918; Paluck, S., Nguyen, T. H., and Maynard, H, D. 2016. “Heparin-mimicking Polymers: Synthesis and Biological Applications,” Biomacromolecules 17(11): 3417-3440; eslie, D. C., Waterhouse, A., Berthet, J. B., Valentin, T. M., Watters, A, L., and 16 others 2014. “A Bioinspired Omniphobic Surface Coating on Medical Devices Prevents Thrombosis and Biofouling,” Nature Biotechnology 32(11): 1134-1140).

If heparin-induced thrombocytopenia is a risk, then non-heparin anticoagulants such as argatroban, bivalirudin with or without aspirin and/or clopidogrel, danaparoid, or fondaparinux are used (see, for example, Schindewolf, M. 2019. “Fondaparinux in Heparin-induced Thrombocytopenia: A Decade's Worth of Clinical Experience,” Research and Practice in Thrombosis and Haemostasis 3(1): 9-11; Glass, C, A. 2018. “Recombinant Heparin—New Opportunities,” Online, Frontiers in Medicine (Lausanne, Switzerland) 5:341; Hensch, L. A., Hui, S. R., and Teruya, J. 2018. “Coagulation and Bleeding Management in Pediatric Extracorporeal Membrane Oxygenation: Clinical Scenarios and Review,” Online, Frontiers in Medicine (Lausanne, Switzerland) 5:361; Linkins. L. A., Hu. G., and Warkentin, T, E. 2018. “Systematic Review of Fondaparinux for Heparin-induced Thrombocytopenia: When There are no Randomized Controlled Trials,” Research and Practice in Thrombosis and Haemostasis 2(4): 678-683; He. Y., He. H., Liu, D., Long. Y., Su, L., and Cheng. W. 2018. “Fondaparinux in a Critically Ill Patient with Heparin-induced Thrombocytopenia: A Case Report,” Medicine (Baltimore, Maryland) 97(37): e12236; Skeith, L. 2018. “Anticoagulating Patients with High-risk Acquired Thrombophilias,” Hematology. American Society of Hematology. Education Program 2018(1): 439-449; Arepally, G. M. 2017. “Heparin-induced Thrombocytopenia,” Blood 129(21): 2864-2872; Sanfilippo, F., Asmussen, S., Maybauer, D. M., Santonocito, C., Fraser, J. F., Erdoes, G., and Maybauer, M. O. 2017. “Bivalirudin for Alternative Anticoagulation in Extracorporeal Membrane Oxygenation: A Systematic Review,” Journal of Intensive Care Medicine 32(5): 312-319; Horlait, G., Minet, V., Mullier, F., and Michaux, I. 2017. “Persistent Heparin-induced Thrombocytopenia: Danaparoid Cross-reactivity or Delayed-onset Heparin-induced Thrombocytopenia? A Case Report,” Blood Coagulation and Fibrinolysis 28(2): 193-197; Maeda, T., Nakagawa, K., Murata, K., Kanaumi, Y., Seguchi, S., and 8 others 2017. “Identifying Patients at High Risk of Heparin-induced Thrombocytopenia-associated Thrombosis with a Platelet Activation Assay Using Flow Cytometry,” Thrombosis and Haemostasis 117(1): 127-138; Minet. V., Dogne, J. M., and Mullier, F. 2017. “Functional Assays in the Diagnosis of Heparin-induced Thrombocytopenia: A Review,” Molecules (Basel, Switzerland) 22(4). pii: E617; Ronchard, T., Salaun, E., Theron, A., Grisoli, D., Jaussaud, N., and 3 others 2017. “Cross-reactivity between Heparin and Danaparoid Antibodies in Cardiac Surgery,” Annals of Thoracic Surgery 103(1): e9-e10; Schindewolf, M., Steindl, J., Beyer-Westendorf, J., Schellong, S., Dohmen, P. M., and 12 others 2017. “Use of Fondaparinux Off-label or Approved Anticoagulants for Management of Heparin-induced Thrombocytopenia,” Journal of the American College of Cardiology 70(21): 2636-2648; Snodgrass, M. N., Shields, J., and Rai, H. 2016. “Efficacy and Safety of Fondaparinux in Patients with Suspected Heparin-induced Thrombocytopenia,” Clinical and Applied Thrombosis/Hemostasis 22(8): 712-717; Benken, S. T., Tillman, N., Dajani, S., Shah, A., and Thomas. T. 2014. “A Retrospective Evaluation of Fondaparinux for Confirmed or Suspected Heparin-induced Thrombocytopenia in Left-ventricular-assist Device Patients,” Journal of Cardiothoracic Surgery 9:55: Grouzi, E. 2014. “Update on Argatroban for the Prophylaxis and Treatment of Heparin-induced Thrombocytopenia Type II,” Journal of Blood Medicine 5:131-141; Pieri, M., Agracheva, N., Bonaveglio, E., Greco, T., De Bonis, M., and 3 others 2013. “Bivalirudin versus Heparin as an Anticoagulant during Extracorporeal Membrane Oxygenation: A Case-control Study,” Journal of Cardiothoracic and Vascular Anesthesia 27(1): 30-34; Nagle, E, L., Dager, W. E., Duby, J. J., Roberts, A. J., Kenny, L. E., Murthy, M. S., and Pretzlaff, R. K. 2013. “Bivalirudin in Pediatric Patients Maintained on Extracorporeal Life Support,” Pediatric Critical Care Medicine 14(4): e182-e188; Babuin, L. and Pengo, V. 2010. “Argatroban in the Management of Heparin-induced Thrombocytopenia,” Vascular Health and Risk Management 6:813-819; Dhillon, S. 2009. “Argatroban: A Review of Its Use in the Management of Heparin-induced Thrombocytopenia,” American Journal of Cardiovascular Drugs 9(4): 261-282).

Because of its immediate response and hesitation to depend upon patient compliance, the postoperatively continued direct pipeline targeting into the coronary arteries of a transplant heart, and/or release into the general circulation of supportive agents, such as an angiotensin II receptor blocker, angiotensin-converting enzyme inhibitor, beta blocker, inotrope, digoxin, hydralazine nitrate, aldosterone antagonist, spironolactone or a diuretic, much less the immunosuppressant, by the implanted sensor-triggered automatic disorder response system can serve to expand the zone of usable replacement hearts and reduce the discarding of those considered less than ideal. For example, this immediate level of support should make it possible to place two transplant hearts, each undersized or otherwise somewhat impaired and thus reduced in ejection fraction, to satisfactorily approximate the work of a normal native heart.

In removing any objection to allowing a plainly essential transplant on the basis that the patient lacks the ability to adhere to the drug regimen essential to sustain the transplant, the response is that the patient will be brought to the clinic for periodic follow-up and implanted drug reservoir replenishment. In this circumstance, the implanted automatic response system can facilitate survival. The hierarchical control system coordinating the treatment of comorbidities and administering the therapy automatically, developments in gene therapy will allow the amelioration if not remediation of symptoms attributable to the underlying genetic defect or defects (see, for example, Ayres, S., Gallacher, L., Stark, Z., and Brett, G, R. 2019. “Genetic Counseling in Pediatric Acute Care: Reflections on Ultra-rapid Genomic Diagnoses in Neonates,” Journal of Genetic Counseling 28(2): 273-282; Brunelli, L., Jenkins, S. M., Gudgeon, J. M., Bleyl, S. B., Miller, C. E., and 15 others 2019. “Targeted Gene Panel Sequencing for the Rapid Diagnosis of Acutely Ill Infants,” Molecular Genetics and Genomic Medicine 7(7): e00796; Clowes Candadai, S. V., Sikes, M. C., Thies, J. M., Freed, A. S., and Bennett, J. T. 2019. “Rapid Clinical Exome Sequencing in a Pediatric ICU: Genetic Counselor Impacts and Challenges,” Journal of Genetic Counseling 28(2): 283-291; Gyngell, C., Newson, A. J., Wilkinson, D., Stark, Z., and Savulescu, J. 2019. “Rapid Challenges: Ethics and Genomic Neonatal Intensive Care,” Pediatrics 143 (Supplement 1): S14-S21; Manolio, T. A., Rowley, R., Williams, M. S., Roden, D., Ginsburg, G. S., and 9 others 2019. “Opportunities, Resources, and Techniques for Implementing Genomics in Clinical Care,” Lancet 394(10197): 511-520; Char, D. S., Lee, S. S., Magnus, D., and Cho, M. 2018. “Anticipating Uncertainty and Irrevocable Decisions: Provider Perspectives on Implementing Whole-genome Sequencing in Critically Ill Children with Heart Disease,” Genetics in Medicine 20(11): 1455-1461; Borghesi, A., Mencarelli, M. A., Memo, L., Ferrero, G. B., Bartuli, A., and 5 others 2017. “Intersociety Policy Statement on the Use of Whole-exome Sequencing in the Critically Ill Newborn Infant,” Italian Journal of Pediatrics 43(1): 100; Char, D. S., Lazaro-Muñoz, G., Barnes, A., Magnus, D., Deem, M. J., and Lantos, J, D. 2017. “Genomic Contraindications for Heart Transplantation,” Pediatrics 139(4). pii: e20163471; Meng, L., Pammi, M., Saronwala, A., Magoulas, P., Ghazi, A. R., and 49 others 2017. “Use of Exome Sequencing for Infants in Intensive Care Units: Ascertainment of Severe Single-gene Disorders and Effect on Medical Management,” Journal of the American Medical Association Pediatrics 171(12): e173438; Petrikin, J. E., Willig, L. K., Smith, L. D., and Kingsmore, S, F. 2015. “Rapid Whole Genome Sequencing and Precision Neonatology,” Seminars in Perinatology 39(8): 623-631).

Briefly addressed below in section 2b(1)(b) entitled Metered Compound vascular bypass, or sudden switched-organ Transplantation, replacement of an impaired heart is through the metered switch technique. If insufficient, the graft heart is reinforced by the addition of a second intact transplant heart. Heterotopic placement is ordinarily used to anatomically merge the better part of a second heart with an impaired native heart, but here the underlying concept, usually applied to overcome pulmonary vascular resistance, is extended to the transplantation of two intact hearts.

A conventional heterotopic (double, ‘piggyback’) heart transplant—not to confused with a piggyback liver transplant-calls for much intracardiac surgery which the switch technique was devised to eliminate; however, the circulatory sufficiency of which two intact hearts, where each can be separately targeted for the release of drugs or electrostimulation, for example, can be made to function in cooperation to exceed the circulatory sufficiency of a conventional heterotopic transplantation.

The automatic system both administers the metered compound vascular bypass, or sudden switched-organ transplantation of the first heart, and thereafter supports both hearts, functioning individually and together as a unit, by instantly releasing routine maintenance or emergency medication directly targeted to either heart through drug delivering catheters according to sensor data received by an implant microcontroller. While the indicia sought exceed those which current cardioverter defibrillators, today often combined pacemaker/cardioverter defibrillators, can provide, these can participate as sensors.

Various implanted sensors allow the measurement of a number of constituents in a passing flow, and if necessary, the shunt can be given an interior coating that aids quantification, such as by attracting a constituent in passing blood up to a ceiling limit that prevents obstruction and allows a sensor to yield real time telemeterable diagnostic data, such as the blood glucose level (see, for example, Huyett, L. M., Mittal, R., Zisser, H. C., Luxon, E. S., Yee, A., Dassau, E., Doyle, F. J. 3rd, and Burnett, D, R. 2016. “Preliminary Evaluation of a Long-Term Intraperitoneal Glucose Sensor with Flushing Mechanism,” Journal of Diabetes Science and Technology 10(5): 1192-1194; Li, Y. J. and Lu, C, C. 2015. “A Novel Scheme and Evaluations on a Long-term and Continuous Biosensor Platform Integrated with a Dental Implant Fixture and Its Prosthetic Abutment,” Online, Sensors (Basel, Switzerland) 15(10:24961-24976).

Before fibroblasts form a capsule about an implant, the foreign body reaction itself causes the adhesion of many blood constituents, allowing these to be quantified (see, for example, Moore, L. B. and Kyriakides, T, R. 2015. “Molecular Characterization of Macrophage-Biomaterial Interactions,” Advances in Experimental Medicine and Biology 865:109-122; Higgins, D. M., Basaraba, R. J., Hohnbaum, A. C., Lee, E. J., Grainger, D. W., and Gonzalez-Juarrero, M. 2009. “Localized Immunosuppressant Environment in the Foreign Body Response to Implanted Biomaterials,” American Journal of Pathology 175(1): 161-170; Anderson, J. M., Rodriguez, A., and Chang, D. T. 2008. “Foreign Body Reaction to Biomaterials,” Seminars in Immunology 20(2): 86-100).

Blood flow measurement does not require such a coating (see, for example, Rothfuss, M. A., Franconi, N. G., Unadkat, J. V., Gimbel, M, L., Star, A., Mickle, M. H., and Sejdic, E. 2016. “A System for Simple Real-Time Anastomotic Failure Detection and Wireless Blood Flow Monitoring in the Lower Limbs,” IEEE [Institute of Electrical and Electronics Engineers] Journal of Translational Engineering in Health and Medicine 4:4100114; Cannata, J. M., Chilipka, T., Yang, H. C., Han, S., Ham, S. W., and 4 others 2012. “Development of a Flexible Implantable Sensor for Postoperative Monitoring of Blood Flow,” Journal of Ultrasound in Medicine 31(11): 1795-1802).

In some cases, the measures incorporated into the implanted automatic response system, to include adaptive servovalve adjustment and the direct targeting of antiarrhythmic drugs, will be sufficient to reverse if not avert a spontaneous desynchronization, so that the need for a cardioverter defibrillator, will be ruled out. Currently the single most effective means for preventing ventricular fibrillation and sudden arrest, a cardioverter defibrillator still carries the risks of infection, premature battery discharge, and inappropriate shock delivery, and the need for replacement once depleted of battery power. Furthermore, with respect to the heart of hearts already transplanted, the implant response system is provided with the resources to deal with any comorbidity or comorbidities that can result in mortality with which a cardioverter defibrillator cannot.

In other cases, the inadvisability of cardioverter defibrillator implantation pertains to hearts which had disease which would have disqualified these for transplantation regardless of the methods to be used. Although the administration of carvedilol can ameliorate the condition, and angiotensin-converting enzyme inhibitors and receptor blockers reduce the incidence of shocks, placed in a donor heart of more severe heart failure and reduced absolute stroke volume, an implantable cardioverter defibrillator can actually prove harmful.

Pacemakers and cardioverter defibrillators would best be superseded by means less likely to damage the heart than through the delivery of powerful (>25 joule) shocks which often warrant ventricular tachycardia or catheter ablation to reduce in frequency (see, for example, Krokhaleva, Y. and Vaseghi, M. 2019. “Update on Prevention and Treatment of Sudden Cardiac Arrest,” Trends in Cardiovascular Medicine 29(7): 394-400; Yousuf, O., Chrispin, J., Tomaselli, G. F., and Berger, R, D. 2015. “Clinical Management and Prevention of Sudden Cardiac Death,” Circulation Research 116(12): 2020-2040). Alternatively, implanting means for averting ventricular fibrillation other than through the discharge of electrical shocks should allow the reduction in frequency and/or intensity of the shocks.

Pacemakers and cardioverter defibrillators can also damage to the myocardium, cause tricuspid valve regurgitation, perforations of a tricuspid valve leaflet, and consequent to lead induced local injury to the myocardium and mistaken sensing, can induce abnormal pacing which can result in ventricular arrhythmias (Puri, M., Chapalamadugu, K. C., Miranda, A. C., Gelot, S., Moreno, W., and 3 others 2013. “Integrated Approach for Smart Implantable Cardioverter Defibrillator (ICD) Device with Real Time ECG Monitoring: Use of Flexible Sensors for Localized Arrhythmia Sensing and Stimulation,” Online, Frontiers in Physiology 4:300). The inducement of a ventricular arrhythmia can also result from pacing of the right ventricle.

Fidgeting with the device when implanted in a subcutaneous pocket by twisting it, known as ‘Twiddler's syndrome; already reported almost a half century ago can dislodge the leads, disabling the device. Overgrowth by tissue can displace the leads and prevent proper sensing. Still other hazards are battery depletion that goes unnotced or unattended and radio frequency, or electromagnetic interference (Puri, M., Chapalamadugu, K. C., Miranda, A. C., Gelot, S., Moreno, W., and 3 others 2013, Op cit., preceding paragraph).

Inappropriate shocks sometimes occur, the heart must have sufficient robustness to withstand these, and beyond a certain level of impairment, the failing heart may lack the wherewithal to respond appropriately to shocks whether appropriate or not. Shortcomings associated with implantable or subcutaneous cardioverter defibrillators in hearts at Class II failure, for example, might argue for the less aggressive and less potentially harmful treatment provided by an implanted response system able to automatically target drugs to the venae cavae according to the onset of action of each and control the diversion and choke valves to adjust the pressures within the heart.

This is because an inherent problem with the use of electrostimulation is that, with preventable causes such as drugs, acidosis, and blood electrolyte and oxygenation imbalances eliminated, the threshold energy of the shocks required to resynchronize a heart is proportional to the degree of impairment; a high defibrillation threshold is associated with more severe failure, the long-term use of antiarrhythmic drugs, and increased risk of sudden cardiac death (see, for example, Biton, Y., Daimee, U. A., Baman, J. R., Kutyifa, V., McNitt, S., and 3 others 2019. “Prognostic Importance of Defibrillator-appropriate Shocks and Antitachycardia Pacing in Patients with Mild Heart Failure,” Journal of the American Heart Association (Oxford, England) 8(6): e010346; Puri, M., Chapalamadugu, K. C., Miranda, A. C., Gelot, S., Moreno, W., and 3 others 2013, Op cit. second paragraph above). The more severely impaired the heart, the greater is the microstructural and electrical anisotropy or discontinuity along the normal paths of conductance through the myocardium, hence, the greater is the chance that electrical propagation will incur interference which only an increase in shock energy can overcome.

The significance and applications of defibrillation threshold test results across a cohort of patients presumes use of the same equipment (see, for example, Friedman, D. J., Parzynski, C. S., Heist, E. K., Russo, A. M., Akar, J. G., and 3 others 2018. “Ventricular Fibrillation Conversion Testing after Implantation of a Subcutaneous Implantable Cardioverter Defibrillator: Report from the National Cardiovascular Data Registry,” Circulation 137(23): 2463-2477; Hayase, J., Do, D. H., and Boyle, N, G. 2018. “Defibrillation Threshold Testing: Current Status,” Arrhythmia and Electrophysiology Review 7(4): 288-293; Sarrazin, J. F., Philippon, F., Sellier, R., Andre, P., O'Hara, G., and 4 others 2018. “Clinical Performance of Different DF-4 Implantable Cardioverter Defibrillator Leads,” Pacing and Clinical Electrophysiology June 1; Bhargava, K. 2014. “DF-4 Lead [to pulse generator] Connector: Innovative Technology, Unexpected Problems and Novel Solutions,” Indian Pacing Electrophysiology Journal 14(3): 108-111; Inbar, S. and Seethala, S. 2014. “A Novel Solution for the High Defibrillation Threshold in Patients with a DF-4 Lead: Adding a High Voltage Adaptor/Splitter,” Indian Pacing and Electrophysiology Journal 14(3): 152-156; Forleo, G. B., Di Biase, L., Mantica, M., Panattoni, G., Santamaria, M., and 7 others 2013. “First Clinical Experience with the New Four-pole Standard Connector for High-voltage ICD Leads. Early Results of a Multicenter Comparison with Conventional Implant Outcomes,” Journal of Interventional Cardiac Electrophysiology 38(1): 11-8; Mond, H. G., Helland, J. R., and Fischer, A. 2013. “The Evolution of the Cardiac Implantable Electronic Device Connector,” Pacing and Clinical Electrophysiology 36(11): 1434-1446).

Threshold testing does not reliably predict sudden arrest, and albeit rarely, carries the risk of errors and adverse effects associated with anesthesia (see, for example, Jacob, S., Pidlaoan, V., Singh, J., Bharadwaj, A., Patel, M. B., and Carrillo, A. 2010. “High Defibrillation Threshold: The Science, Signs and Solutions,” Indian Pacing and Electrophysiology Journal 10(1): 21-39; Weinbroum, A. A., Glick, A., Copperman, Y., Yashar, T., Rudick, V., and Flaishon, R. 2002. “Halothane, Isoflurane, and Fentanyl Increase the Minimally Effective Defibrillation Threshold of an Implantable Cardioverter Defibrillator: First Report in Humans,” Anesthesia and Analgesia 95(5): 1147-1153; Cohen, T. J., Chengot, T., Quan, C., and Peller, A. P. 2000. “Elevation of Defibrillation Thresholds with Propofol during Implantable Cardioverter-defibrillator Testing,” Journal of Invasive Cardiology 12(2): 121-123; Horvath, G., Ilan, M., Kadish, A., and Goldberger, J. 1999. “Effect of Isoflurane on Defibrillation Threshold in Biphasic Active—can Defibrillation Systems,” Journal of Invasive Cardiology 11(11): 700-702; Stix, G., Anvari, A., Podesser, B., Pernerstorfer, T., Mayer, C., Laufer, G., and Schmidinger, H. 1999. “Local Anaesthesia versus General Anaesthesia for Cardioverter-defibrillator Implantation,” Wiener Klinische Wochenschrift 111(10): 406-409) and the possibility of provoking an arrest, is not as significant as the fact that when correctly performed (see, for example, Roos, M., Geller, J. C., and Ohlow, M, A. 2017. “Critical Analysis of Ineffective Post Implantation Implantable Cardioverter-defibrillator-testing,” World Journal of Cardiology 9(2): 167-173; Ohlow, M. A., Roos, M., Lauer, B., and Geller, J, C. 2016. “Incidence of Ineffective Safety Margin Testing (<10 J) and Efficacy of Routine Subcutaneous Array Insertion during Implantable Cardioverter Defibrillator Implantation,” Indian Pacing and Electrophysiology Journal 16(2): 47-52)

Injury-free, the results, as supplementary to an electrocardiogram, are obtained with a patient in need of an implanted defibrillator passively, rather than by exercise stress testing, to provide an additional indication as to whether the cardioverter defibrillator should be considered a short term bridging device and the patient placed on the waiting list for a heart. While implantable cardioverter defibrillators undoubtedly save many lives, defibrillation threshold testing before implantation of a cardioverter-defibrillator appears to have no effect on mortality.

In the treatment of a failing native heart, rather than apportioning venous return volume between either of two hearts as in a double heart transplant, diversion servovalves can tap off to or draw in blood from neighboring larger vessels and/or choke valves contract or open to increase to decrease more central or intracardiac pressures. Provided with suitable sensors and actuators, an additional approach with the potential to terminate ventricular arrhythmias without the potential to damage the heart as can the shocks from an implantable cardioverter defibrillator and which can be automatically administered by the implanted automatic disorder response system is based upon optogenetics, currently experimental and under study.

One object in developing this technology is to reduce in number and/or intensity if not eliminate the shocks needed to achieve resynchronization. The diagnostic and therapeutic application of optogenetics to medical practice will require the development of less immunologically provocative opsins, reduced heat generation, and techniques for introducing the microbial (viral) opsin, for example, into the structure to be target-infected precisely and more quickly than has been accomplished this early in its development. Heart specific references are cited below in this section. The tiny light-emitting diodes are implanted to the sides of the infected cells in the brainstem for control by the microcontroller assigned to that level in the hierarchy or directly by the master control microprocessor.

Neural circuit selective optogenetics and electronic targeting is under intensive study (see, for example, Alkawadri, R. 2019. “Brain-Computer Interface (BCI) Applications in Mapping of Epileptic Brain Networks Based on Intracranial-EEG: An Update,” Online, Frontiers in Neuroscience 13:191; Anderson, H E. and Weir, R, F. F. 2019. “On the Development of Optical Peripheral Nerve Interfaces,” Neural Regeneration Research 14(3): 425-436; Deep. N, L., Choudhury, B., and Roland. J. T. Jr. 2019. “Auditory Brainstem Implantation: An Overview,” Journal of Neurological Surgery. Part B. Skull Base 80(2): 203-208; Williams, J. J., Watson, A. M., Vazquez, A, L., and Schwartz, A, B. 2019. “Viral-mediated Optogenetic Stimulation of Peripheral Motor Nerves in Non- human Primates,” Online, Frontiers in Neuroscience 13:759; Wong, K., Kozin, E. D., Kanumuri, V V., Vachicouras, N., Miller, J., and 3 others 2019. “Auditory Brainstem Implants: Recent Progress and Future Perspectives,” Online, Frontiers in Neuroscience 13:10; Camporeze, B., Manica, B. A., Bonafe, G. A., Ferreira, J. J. C., Diniz, A, L., and 4 others 2018. “Optogenetics: The New Molecular Approach to Control Functions of Neural Cells in Epilepsy, Depression and Tumors of the Central Nervous System,” American Journal of Cancer Research 8(10): 1900-1918; Maimon, B. E., Diaz, M., Revol, E, C. M., Schneider, A. M., Leaker, B., and 4 others 2018. “Optogenetic Peripheral Nerve Immunogenicity,” (Nature Research) Scientific Reports 8(1): 14076; Mattingly, M., Weineck, K., Costa, J., and Cooper, R. L. 2018. “Hyperpolarization by Activation of Halorhodopsin Results in Enhanced Synaptic Transmission: Neuromuscular Junction and CNS Circuit,” Online, PLoS [Public Library of Science] One 13(7): e0200107; Delbeke, J., Hoffman, L., Mols, K., Braeken, D., and Prodanov, D. 2017. “And Then There Was Light: Perspectives of Optogenetics for Deep Brain Stimulation and Neuromodulation,” Online, Frontiers in Neuroscience 11:663; Mahmoudi, P., Veladi, H., and Pakdel, F, G. 2017. “Optogenetics, Tools and Applications in Neurobiology,” Journal of Medical Signals and Sensors 7(2): 71-79; Ordaz, J. D., Wu. W., and Xu, X. M. 2017. “Optogenetics and Its Application in Neural Degeneration and Regeneration,” Neural Regeneration Research 12(8): 1197-1209; Cheng, M. Y., Aswendt. M., and Steinberg, G. K. 2016. “Optogenetic Approaches to Target Specific Neural Circuits in Post-stroke Recovery,” Neurotherapeutics 13(2): 325-340; Park, H, G. and Carmel, J, B. 2016. “Selective Manipulation of Neural Circuits,” Neurotherapeutics 13(2): 311-324; Glock, C., Nagpal, J., and Gottschalk, A. 2015. “Microbial Rhodopsin Optogenetic Tools: Application for Analyses of Synaptic Transmission and of Neuronal Network Activity in Behavior,” Methods in Molecular Biology 1327:87-103; Hight, A. E., Kozin, E. D., Darrow, K., Lehmann, A., Boyden, E., Brown, M. C., and Lee, D. J. 2015. “Superior Temporal Resolution of Chronos versus Channelrhodopsin-2 in an Optogenetic Model of the Auditory Brainstem Implant,” Hearing Research 322:235-241; Zhao, M., Alleva, R., Ma, H., Daniel, A. G., and Schwartz, T, H. 2015. “Optogenetic Tools for Modulating and Probing the Epileptic Network,” Epilepsy Research 116:15-26; Costa, W. S., Liewald. J., and Gottschalk, A. 2014. “Photoactivated Adenylyl Cyclases as Optogenetic Modulators of Neuronal Activity,” Methods in Molecular Biology 1148:161-175; Nikolic, K., Jarvis, S., Grossman, N., and Schultz, S. 2013. “Computational Models of Optogenetic Tools for Controlling Neural Circuits with Light,” Proceedings of the Annual International Conference of the Institute of Electrical and Electronics Engineers Engineering in Medicine and Biology Society 2013:5934-5937).

The assistance if not replacement of current means for life support by optogenetic stimulation of the brainstem control centers governing cardiorespiratory function should allow organ sustainment over a longer term. Optogenetic neuromodulation is also pertinent to means for assisting control of the urinary bladder, addressed below in section 2c, entitled Ductus Side-entry Jackets and Vascular Valves in Urinary Diversion

Once sufficiently refined for application in the clinic, this technology can supplement if not supplant the need for an implanted cardioverter defibrillator which is limited to electrostimulation, or electrode shock based cardiac resynchronization therapy and the use of antiarrhythmic drugs (see, for example, Ambrosi, C. M., Sadananda, G., Han, J, L., and Entcheva, E. 2019. “Adeno-associated Virus Mediated Gene Delivery: Implications for Scalable in Vitro and in Vivo Cardiac Optogenetic Models,” Online, Frontiers in Physiology 10:168; Dong. R., Mu-U-Min, R., Reith, A. J. M., O'Shea, C., He. S., and 7 others 2019. “A Protocol for Dual Calcium-Voltage Optical Mapping in Murine Sinoatrial Preparation with Optogenetic Pacing,” Online, Frontiers in Physiology 10:954; Funken, M., Malan, D., Sasse, P., and Bruegmann, T. 2019. “Optogenetic Hyperpolarization of Cardiomyocytes Terminates Ventricular Arrhythmia,” Online, Frontiers in Physiology 10:498; Liu S., Tang. Y., Xing. Y., Kramer, P., Bellinger, L., and Tao, F. 2019. “Potential Application of Optogenetic Stimulation in the Treatment of Pain and Migraine Headache: A Perspective from Animal Studies,” Brain Sciences (Basel, Switzerland) 9(2). pii: E26; Mickle, A. D., Won, S. M., Noh, K. N., Yoon, J., Meacham, K. W., and 20 others 2019. “A Wireless Closed-loop System for Optogenetic Peripheral Neuromodulation,” Nature 565(7739): 361-365; Moreno, A., Endicott, K., Skancke, M., Dwyer, M. K., Brennan, J., and 4 others 2019. “Sudden Heart Rate Reduction upon Optogenetic Release of Acetylcholine from Cardiac Parasympathetic Neurons in Perfused Hearts,” Online, Frontiers in Physiology 10:16; O'Shea, C., Holmes, A. P., Winter, J., Correia, J., Ou X., and 6 others 2019. “Cardiac Optogenetics and Optical Mapping-Overcoming Spectral Congestion in All-optical Cardiac Electrophysiology,” Online, Frontiers in Physiology 10:182; Rajendran, P. S., Challis, R. C., Fowlkes, C. C., Hanna, P., Tompkins, J. D., and 11 others 2019. “Identification of Peripheral Neural Circuits that Regulate Heart Rate Using Optogenetic and Viral Vector Strategies,” Nature Communications 10(1): 1944; Richter, C. and Bruegmann, T. 2019. “No Light without the Dark: Perspectives and Hindrances for Translation of Cardiac Optogenetics,” Progress in Biophysics and Molecular Biology September 9. pii: S0079-6107(19) 30114-2; Sasse, P., Funken, M., Beiert. T., and Bruegmann, T. 2019. “Optogenetic Termination of Cardiac Arrhythmia: Mechanistic Enlightenment and Therapeutic Application?,” Online, Frontiers in Physiology 10:675; Waldron, N. H., Fudim, M., Mathew, J. P., and Piccini, J. P. 2019. “Neuromodulation for the Treatment of Heart Rhythm Disorders,” Journal of the American College of Cardiology. Basic to Translational Science 4(4): 546-562; Zaglia. T., Di Bona, A., and Mongillo, M. 2019. “A Light Wand to Untangle the Myocardial Cell Network,” Methods and Protocols (Basel, Switzerland) 2(2). pii: E34; Zhang, Y., Castro, D. C., Han, Y., Wu. Y., Guo, H., and 25 others 2019. “Battery-free, Lightweight, Injectable Microsystem for in Vivo Wireless Pharmacology and Optogenetics,” Online, Proceedings of the National Academy of Sciences of the United States of America 116(43): 21427-21437; Zhang. Y., Mickle, A. D., Gutruf, P., Mellvried, L. A., Guo, H., and 15 others 2019. “Battery-free, Fully Implantable Optofluidic Cuff System for Wireless Optogenetic and Pharmacological Neuromodulation of Peripheral Nerves,” Science Advances 5(7): eaaw5296; Boyle, P. M., Karathanos, T. V., and Trayanova, N, A. 2018. “Cardiac Optogenetics: 2018,” Journal of the American College of Cardiology. Clinical Electrophysiology 4(2): 155-167; Boyle, P. M., Murphy, M. J., Karathanos, T. V., Zahid, S., Blake, R, C. 3rd, and Trayanova, N, A. 2018. “Termination of Re-entrant Atrial Tachycardia via Optogenetic Stimulation with Optimized Spatial Targeting: Insights from Computational Models,” Journal of Physiology 596(2): 181-196; Bruegmann, T., Beiert. T., Vogt, C. C., Schrickel, J. W., and Sasse, P. 2018. “Optogenetic Termination of Atrial Fibrillation in Mice,” Cardiovascular Research 114(5): 713-723; Edward, E. S., Kouzani, A. Z., and Tye, S. J. 2018. “Towards Miniaturized Closed-loop Optogenetic Stimulation Devices,” Journal of Neural Engineering 15(2): 021002; Goncalves, S. B., Ribeiro, J. F., Silva, A. F., Costa, R. M., and Correia, J, H. 2017. “Design and Manufacturing Challenges of Optogenetic Neural Interfaces: A Review,” Journal of Neural Engineering 14(4): 041001; Gruber, A., Edri. O., and Gepstein, L. 2018. “Cardiac Optogenetics: The Next Frontier,” Europace 20(12): 1910-1918; Iseri, E. and Kuzum, D. 2017. “Implantable Optoelectronic Probes for in Vivo Optogenetics,” Journal of Neural Engineering 14(3): 031001; Koopman. C. D., Zimmermann, W. H., Knöpfel. T., and de Boer, T. P. 2017. “Cardiac Optogenetics: Using Light to Monitor Cardiac Physiology,” Basic Research in Cardiology 112(5): 56: Nyns, E, C. A., Kip, A., Bart, C. I., Plomp, J. J., Zeppenfeld. K., and 3 others 2017. “Optogenetic Termination of Ventricular Arrhythmias in the Whole heart: Towards Biological Cardiac Rhythm Management,” European Heart Journal 38(27): 2132-2136; Pianca, N., Zaglia. T., and Mongillo, M. 2017. “Will Cardiac Optogenetics Find the Way through the Obscure Angles of Heart Physiology?,” Biochemical and Biophysical Research Communications 482(4): 515-523; Rudmann, L., Alt, M. T., Ashouri Vajari, D., and Stieglitz, T. 2018. “Integrated Optoelectronic Microprobes,” Current Opinion in Neurobiology 50:72-82; Watanabe, M., Feola, I., Majumder. R., Jangsangthong, W., Teplenin, A. S., and 5 others 2017. “Optogenetic Manipulation of Anatomical Re-entry by Light-guided Generation of a Reversible Local Conduction Block,” Cardiovascular Research 113(3): 354-366; Bruegmann, T., Boyle, P. M., Vogt, C. C., Karathanos, T. V., Arevalo, H. J., and 3 others 2016. “Optogenetic Defibrillation Terminates Ventricular Arrhythmia in Mouse Hearts and Human Simulations,” Journal of Clinical Investigation 126(10): 3894-3904; Entcheva, E. and Bub, G. 2016. “All-optical Control of Cardiac Excitation: Combined High-resolution Optogenetic Actuation and Optical Mapping,” Journal of Physiology 594(9): 2503-2510; Karathanos, T. V., Bayer, J. D., Wang. D., Boyle, P. M., and Trayanova, N, A. 2016. “Opsin Spectral Sensitivity Determines the Effectiveness of Optogenetic Termination of Ventricular Fibrillation in the Human Heart: A Simulation Study,” Journal of Physiology 594(23): 6879-6891; Liu. S., Li, C., Xing. Y., Wang. Y., and Tao, F. 2016. “Role of Neuromodulation and Optogenetic Manipulation in Pain Treatment,” Current Neuropharmacology 14(6): 654-661; Liu, S. and Tao, F. 2016. “Application of Optogenetics-mediated Motor Cortex Stimulation in the Treatment of Chronic Neuropathic Pain,” Journal of Translational Science 2(5): 286-288; Richter, C., Christoph, J., Lehnart, S. E., and Luther, S. 2016. “Optogenetic Light Crafting Tools for the Control of Cardiac Arrhythmias,” Methods in Molecular Biology (Clifton, New Jersey) 1408:293-302; Boyle P. M., Karathanos, T. V., and Trayanova, N, A. 2015. “Beauty is a Light in the Heart: The Transformative Potential of Optogenetics for Clinical Applications in Cardiovascular Medicine,” Trends in Cardiovascular Medicine 25(2): 73-81; Fan, B. and Li, W. 2015, Op cit.; Wengrowski, A. M., Wang, X., Tapa, S., Posnack, N. G., Mendelowitz, D., and Kay, M. W. 2015. “Optogenetic Release of Norepinephrine from Cardiac Sympathetic Neurons Alters Mechanical and Electrical Function,” Cardiovascular Research 105(2): 143-150; Ambrosi, C. M1, and Entcheva, E. 2014. “Optogenetic Control of Cardiomyocytes via Viral Delivery,” Methods in Molecular Biology (Clifton, New Jersey) 1181:215-228; Ambrosi, C. M., Klimas, A., Yu, J., and Entcheva, E. 2014. “Cardiac Applications of Optogenetics,” Progress in Biophysics and Molecular Biology 115(2-3): 294-304; Bingen, B. O., Engels, M. C., Schalij. M. J., Jangsangthong, W., Neshati, Z., and 6 others 2014. “Light-induced Termination of Spiral Wave Arrhythmias by Optogenetic Engineering of Atrial Cardiomyocytes,” Cardiovascular Research 104(1): 194-205; Klimas, A. and Entcheva, E. 2014. “Toward Microendoscopy-inspired Cardiac Optogenetics in Vivo: Technical Overview and Perspective,” Journal of Biomedical Optics (Bellingham, Washington) 19(8): 080701; Trayanova, N, A. and Boyle, P. M. 2014. “Advances in Modeling Ventricular Arrhythmias: From Mechanisms to the Clinic,” Wiley Interdisciplinary Reviews. Systems Biology and Medicine 6(2): 209-224; Abilez, O. J. 2013. “Optogenetic LED Array for Perturbing Cardiac Electrophysiology,” Annual Conference Proceedings of the Institute of Electrical and Electronics Engineers Engineering in Medicine and Biology Society 2013:1619-1622; Entcheva, E. 2013. “Cardiac Optogenetics,” American Journal of Physiology, Heart and Circulatory Physiology 304(9): H1179-H1191; Kalanithi, P. S. and Henderson, J. M. 2012. “Optogenetic Neuromodulation,” International Review of Neurobiology 107:185-205).

Optogenetics has been combined with stem cell therapy in the repair of nervous tissue, which an implanted automatic disorder response system could administer (Yu. S. P., Tung, J. K., Wei, Z. Z., Chen, D., Berglund, K., and 7 others 2019. “Optochemogenetic Stimulation of Transplanted iPS-NPCs [induced pluripotent stem cell-derived neural progenitor cells] Enhances Neuronal Repair and Functional Recovery after Ischemic Stroke,” Journal of Neuroscience 39(33): 6571-6594). The energy requirements to power an artificial heart are sufficiently onerous that work over decades on an implantable nuclear powered steam engine to power an artificial heart was eventually abandoned due to a predictable buildup of heat and the certainty of radiation poisoning (references cited below in section 2b(1)(b)(b5), entitled Heterotopic Double Heart Metered Compound Vascular Bypass, or Switched-organ, Transplantation [and so on].

Seeking the maximum benefit with the least expenditure of energy, hence, the longest service life and interval for recharging or replacement of a pacemaker/cardioverter defibrillator, yet another approach to cardiac electrical neuromodulation targets the cardiac autonomic nervous system at multiple levels, to include vagal, stellate ganglion, renal nerves, and spinal cord. Alternative means of electrical neuromodulation may eliminate the need for a potentially injurious shock-emitting cardioverter defibrillator, or supplementary means allow a reduction in the defibrillation threshold energy of the shocks.

An automatic implant system can implement and coordinate the technology with the release of drugs each based upon its onset of action (see, for example, Dusi, V., Zhu, C., and Ajijola, O. A. 2019. “Neuromodulation Approaches for Cardiac Arrhythmias: Recent Advances,” Current Cardiology Reports 21(5): 32; Kiuchi, M. G., Nolde, J. M., Villacorta, H., Camagarin, R., Chan, J. J. S., and 5 others 2019. “New Approaches in the Management of Sudden Cardiac Death in Patients with Heart Failure-targeting the Sympathetic Nervous System,” Online, MDPI [Multidisciplinary Digital Publishing Institute] International Journal of Molecular Sciences (Basel, Switzerland) 20(10). pii: E2430; Lai, Y., Yu, L., and Jiang, H. 2019. “Autonomic Neuromodulation for Preventing and Treating Ventricular Arrhythmias,” Online, Frontiers in Physiology 10:200; Waldron, N. H., Fudim, M., Mathew, J. P., and Piccini, J. P. 2019. “Neuromodulation for the Treatment of Heart Rhythm Disorders,” Journal of the American College of Cardiology. Basic to Translational Science 4(4): 546-562; Wang, Z., Li, S., Lai, H., Zhou, L., Meng. G., and 6 others 2019. “Interaction between Endothelin-1 and Left Stellate Ganglion Activation: A Potential Mechanism of Malignant Ventricular Arrhythmia during Myocardial Ischemia,” Oxidative Medicine and Cellular Longevity 2019:6508328; Zhu, C., Hanna, P., Rajendran, P. S., and Shivkumar, K. 2019. “Neuromodulation for Ventricular Tachycardia and Atrial Fibrillation: A Clinical Scenario-based Review,” Journal of the American College of Cardiology. Clinical Electrophysiology 5(8): 881-896; Bradfield, J. S., Ajijola, O. A., Vaseghi, M., and Shivkumar, K. 2018. “Mechanisms and Management of Refractory Ventricular Arrhythmias in the Age of Autonomic Modulation,” Heart Rhythm 15(8): 1252-1260; Meng, L., Shivkumar, K., and Ajijola, O. 2018. “Autonomic Regulation and Ventricular Arrhythmias,” Current Treatment Options Cardiovascular Medicine 20(5): 38; Wang, L., Song, L., Li, C., Feng, Q., Xu. M., Li, Z., and Lu, C. 2018. “Renal Denervation Improves Cardiac Function by Attenuating Myocardiocyte Apoptosis in Dogs after Myocardial Infarction,” Online, BioMed Central Cardiovascular Disorders 18(1): 86; Yu, L., Wang. Y., Zhou, X., Huang, B., Wang, M., and 5 others 2018. “Leptin Injection into the Left Stellate Ganglion Augments Ischemia-related Ventricular Arrhythmias via Sympathetic Nerve Activation,” Heart Rhythm 15(4): 597-606; Feng, Q., Lu, C., Wang, L., Song, L., Li, C., and Uppada, R, C. 2017. “Effects of Renal Denervation on Cardiac Oxidative Stress and Local Activity of the Sympathetic Nervous System and Renin-angiotensin System in Acute Myocardial Infracted Dogs,” Online, BioMed Central Cardiovascular Disorders 17(1): 65; Huang, W. A., Boyle, N. G., and Vaseghi, M. 2017. “Cardiac Innervation and the Autonomic Nervous System in Sudden Cardiac Death” Cardiac Electrophysiology Clinics 9(4): 665-679; Irie, T., Yamakawa, K., Hamon, D., Nakamura, K., Shivkumar, K., and Vaseghi, M. 2017. “Cardiac Sympathetic Innervation via Middle Cervical and Stellate Ganglia and Antiarrhythmic Mechanism of Bilateral Stellectomy,” American Journal of Physiology, Heart and Circulatory Physiology 312(3): H392-H405; Jackson, N., Gizurarson, S., Azam, M. A., King, B., Ramadeen, A., 13 others 2017. “Effects of Renal Artery Denervation on Ventricular Arrhythmias in a Postinfarct Model,” Circulation. Cardiovascular Interventions 10(3): e004172; Witt, C. M., Bolona, L., Kinney, M. O., Moir, C., Ackerman, M. J., Kapa, S., Asirvatham, S., and McLeod, C. J. 2017. “Denervation of the Extrinsic Cardiac Sympathetic Nervous System as a Treatment Modality for Arrhythmia,” Europace 19(7): 1075-1083; Hou. Y., Zhou, Q., and Po. S. S. 2016. “Neuromodulation for Cardiac Arrhythmia,” Heart Rhythm 13(2): 584-592; Ng. G, A. 2016. “Neuro-cardiac Interaction in Malignant Ventricular Arrhythmia and Sudden Cardiac Death,” Autonomie Neuroscience: Basic and Clinical 199:66-79; Zheng. X. X., Li, X. Y., Lyu. Y. N., He, Y. Y., Wan, W. G., Zhu, H, L., Jiang, X. J. 2016. “Possible Mechanism by which Renal Sympathetic Denervation Improves Left Ventricular Remodelling after Myocardial Infarction,” Experimental Physiology 101(2): 260-271; Fukuda, K., Kanazawa, H., Aizawa, Y., Ardell, J, L., and Shivkumar, K. 2015. “Cardiac Innervation and Sudden Cardiac Death,” Circulation Research 116(12): 2005-2019; Huang. B., Scherlag, B. J., Yu, L., Lu, Z., He. B., and Jiang, H. 2015. “Renal Sympathetic Denervation for Treatment of Ventricular Arrhythmias: A Review on Current Experimental and Clinical Findings,” Clinical Research in Cardiology 104(7): 535-543; Hucker, W. J., Singh, J. P., Parks, K., and Armoundas, A, A. 2014. “Device-based Approaches to Modulate the Autonomic Nervous System and Cardiac Electrophysiology,” Arrhythmia and Electrophysiology Review 3(1): 30-35; Shen. M. J. and Zipes, D. P. 2014. “Role of the Autonomic Nervous System in Modulating Cardiac Arrhythmias,” Circulation Research 114(6): 1004-1021; Huang, B., Yu, L., Scherlag, B. J., Wang, S., He, B., and 7 others 2014. “Left Renal Nerves Stimulation Facilitates Ischemia-induced Ventricular Arrhythmia by Increasing Nerve Activity of Left Stellate Ganglion,” Journal of Cardiovascular Electrophysiology 25(11): 1249-1256).

Optogenetic and autonomic means may not supplant the function of a cardioverter defibrillator but allow its less intensive and less potentially damaging use by reducing the threshold energy required to reinstate a normal sinus rhythm with lower intensity shocks (see, for example, Semenov, I., Grigoryev, S., Neuber, J. U., Zemlin, C. W., Pakhomova, O. N., Casciola, M., and Pakhomov. A, G. 2018. “Excitation and Injury of Adult Ventricular Cardiomyocytes by Nano- to Millisecond Electric Shocks,” (Nature Research) Scientific Reports 8(1): 8233; Stoevelaar, R., Brinkman- Stoppelenburg, A., Bhagwandien, R. E., van Bruchem-Visser, R, L., Theuns, D. A., van der Heide, A., and Rietjens, J, A. 2018. “The Incidence and Impact of Implantable Cardioverter Defibrillator Shocks in the Last Phase of Life: An Integrated Review,” European Journal of Cardiovascular Nursing 17(6): 477-485; Peterson, P. N., Greenlee, R. T., Go, A. S., Magid, D. J., Cassidy-Bushrow, A., and 14 others 2017. “Comparison of Inappropriate Shocks and Other Health Outcomes between Single- and Dual-chamber Implantable Cardioverter-Defibrillators for Primary Prevention of Sudden Cardiac Death: Results from the Cardiovascular Research Network Longitudinal Study of Implantable Cardioverter-Defibrillators,” Journal of the American Heart Association (Oxford, England) 6(11). pii: e006937; Kinch Westerdahl, A., Sjöblom, J., Mattiasson, A. C., Rosenqvist, M., and Frykman, V. 2014. “Implantable Cardioverter-Defibrillator Therapy before Death: High Risk for Painful Shocks at End of Life,” Circulation 129(4): 422-429; Miranda, C. H., Schmidt, A., and Pazin-Filho, A. 2014. “Prognostic Evaluation of the Troponin I Elevation after Multiple Spontaneous Shocks of the Implantable Cardioverter/Defibrillator,” American Journal of Emergency Medicine 32(9): 1085-1088; Sharn'a, R. A., Nery, P., Sadek. M., Yung, D., Redpath, C., and 3 others 2014. “Myocardial Injury Secondary to ICD Shocks: Insights from Patients with Lead Fracture,” Pacing and Clinical Electrophysiology 37(2): 237-241; Walcott, G. P., Killingsworth, C. R., and Ideker, R, E. 2003. “Do Clinically Relevant Transthoracic Defibrillation Energies Cause Myocardial Damage and Dysfunction?,” Resuscitation 59(1): 59-70). Such a heart would never be chosen for implantation; indeed, it would be targeted for replacement.

Seen thus, in more severely impaired hearts, the combination of immediately responsive diversion servovalve adjustments and automatic delivery of supportive medication in a heart not yet scheduled for replacement would likely prove superior to the use of an electrostimulator or the use thereof not combined with medicinal support. This pertains exclusively to treatment by the automatic implant response system of a native heart at stage IV failure, not one that would be considered for transplantation. For hearts with a defibrillation too high for the defibrillator to dispel, strategic positioning of the device may remedy the shortfall (Mitra, R. L. 2018. “Left Axillary Active Can Positioning Markedly Reduces Defibrillation Threshold of a Transvenous Defibrillator Failing to Defibrillate at Maximum Output” HeartRhythm Case Reports 5(1): 36-39).

While implantable cardioverter defibrillators undoubtedly save many lives, defibrillation threshold testing before implantation of a cardioverter-defibrillator appears to have no effect on mortality (Kannabhiran, M., Mustafa, U., Acharya, M., Telles, N., Alexandria, B., Reddy, P., and Dominic, P. 2018. “Routine DFT [defibrillation threshold] Testing in Patients undergoing ICD Implantation Does Not Improve Mortality: A Systematic Review and Meta-analysis,” Journal of Arrhythmia 34(6): 598-606; Arnson, Y., Suleiman, M., Glikson, M., Sela, R., Geist, M., and 9 others 2014. “Role of Defibrillation Threshold Testing during Implantable Cardioverter-defibrillator Placement: Data from the Israeli ICD Registry,” Heart Rhythm 11(5): 814-821; Russo, A. M. and Chung, M. K. 2014. “Is Defibrillation Testing Necessary?,” Cardiology Clinics 32(2): 211-224; Aktas, M. K., Huang, D. T., Daubert, J. P., Schuger, C. D., MeNitt, S., and 3 others 2013. “Effect of Defibrillation Threshold Testing on Heart Failure Hospitalization or Death in the Multicenter Automatic Defibrillator Implantation Trial-cardiac Resynchronization Therapy (MADIT-CRT),” Heart Rhythm 10(2): 193-199).

Unable to address an eventuality that laid in the future, the literature on cardioverter defibrillators still furnishes sufficient electrophysiological and pharmaceutical information to serve as a basis for planning double heart transplantations (see for example, Ayan, M., Habash, F., Alqam. B., Gheith, Z., Cross. M., Vallurupalli, S., and Paydak, H. 2019. “A Comparison of Anti-arrhythmic Efficacy of Carvedilol vs Metoprolol Succinate in Patients with Implantable Cardioverter-Defibrillators,” Clinical Cardiology 42(2): 299-304; Bazoukis, G., Tse. G., Korantzopoulos, P., Liu. T., Letsas, K. P., Stavrakis, S., and Naka, K. K. 2019. “Impact of Implantable Cardioverter-Defibrillator Interventions on All-cause Mortality in Heart Failure Patients: A Meta-analysis,” Cardiology in Review 27(3): 160-166; Merchant, F. M., Desai, Y., Addish, M. A., Kelly, K., Casey, M., and 3 others 2018. “Implantable Cardioverter-Defibrillator Placement for Primary Prevention in 2,346 Patients: Predictors of One-year Survival,” Texas Heart Institute Journal 45(4): 221-225; Bilchick, K. C., Wang. Y., Cheng. A., Curtis, J. P., Dharmarajan, K., and 10 others 2017. “Seattle Heart Failure and Proportional Risk Models Predict Benefit from Implantable Cardioverter-Defibrillators,” Journal of the American College of Cardiology 69(21): 2606-2618; Brewster, J., Sexton. T., Dhaliwal. G., Chamigo, R., Morales, G., and 5 others 2017. “Acute Effects of Implantable Cardioverter-Defibrillator Shocks on Biomarkers of Myocardial Injury, Apoptosis, Heart Failure, and Systemic Inflammation,” Pacing and Clinical Electrophysiology40(4): 344-352; Kaura, A., Sunderland, N., Kamdar, R., Petzer, E., McDonagh, T., and 3 others 2017. “Identifying Patients with Less Potential to Benefit from Implantable Cardioverter-defibrillator Therapy: Comparison of the Performance of Four Risk Scoring Systems,” Journal of Interventional Cardiac Electrophysiology 49(2): 181-189; Peterson, P. N., Greenlee, R. T., Go, A. S., Magid, D. J., Cassidy-Bushrow, A., and 14 others 2017. “Comparison of Inappropriate Shocks and Other Health Outcomes between Single- and Dual-chamber Implantable Cardioverter-Defibrillators for Primary Prevention of Sudden Cardiac Death: Results from the Cardiovascular Research Network Longitudinal Study of Implantable Cardioverter-Defibrillators,” Journal of the American Heart Association (Oxford, England) 6(11). pii: e006937; Pontone, G., Guaricci, A. I., Andreini, D., Solbiati, A., Guglielmo, M., and 18 others 2016. “Prognostic Benefit of Cardiac Magnetic Resonance over Transthoracic Echocardiography for the Assessment of Ischemic and Nonischemic Dilated Cardiomyopathy Patients Referred for the Evaluation of Primary Prevention Implantable Cardioverter-defibrillator Therapy,” Circulation. Cardiovascular Imaging 9(10). pii: e004956; Qian, Z., Zhang, Z., Guo, J., Wang. Y., Hou, X., Feng. G., and Zou, J. 2016. “Association of Implantable Cardioverter Defibrillator Therapy with All-cause Mortality-A Systematic Review and Meta-analysis,” Pacing and Clinical Electrophysiology 39(1): 81-88; AlJaroudi [Al Jaroudi]. W. A., Refaat, M. M., Habib, R. H., Al-Shaar, L., Singh, M., and 10 others 2015. “Effect of Angiotensin-converting Enzyme Inhibitors and Receptor Blockers on Appropriate Implantable Cardiac Defibrillator Shock in Patients with Severe Systolic Heart Failure (from the GRADE Multicenter Study),” Online, American Journal of Cardiology 115(7): 924-931; Zhang, Y., Guallar, E., Blasco-Colmenares, E., Dalal. D., Butcher, B., and 8 others 2015. “Clinical and Serum-based Markers are Associated with Death within 1 Year of de Novo Implant in Primary Prevention ICD Recipients,” Heart Rhythm 12(2): 360-366; Cheng. A., Zhang, Y., Blasco-Colmenares. E., Dalal. D., Butcher, B., and 8 others 2014. “Protein Biomarkers Identify Patients Unlikely to Benefit from Primary Prevention Implantable Cardioverter Defibrillators: Findings from the Prospective Observational Study of Implantable Cardioverter Defibrillators (PROSE-ICD),” Circulation. Arrhythmia and Electrophysiology 7(6): 1084-1091; Disertori, M., Quintarelli, S., Mazzola, S., Favalli, V., Narula, N., and Arbustini, E. 2013. “The Need to Modify Patient Selection to Improve the Benefits of Implantable Cardioverter-defibrillator for Primary Prevention of Sudden Death in Non-ischaemic Dilated Cardiomyopathy,” Europace 15(12): 1693-701).

The use of the implanted cardioverter defibrillator as a primary sensor to signal the master controller of arrhythmogenic events, which in the context of two rather than just one heart, warrant adjuvant treatment consisting of valve adjustments and remediation through the directly targeted release of drugs to the affected heart or hearts, is by connection to the defibrillator cardiac sensors, which in improved defibrillators will include a WiFi flexible cardiac sensor array to transmit not just the mere fact of defibrillation but the exact location thereof (Puri, M., Chapalamadugu, K. C., Miranda, A. C., Gelot, S., Moreno, W., and 3 others 2013. Op cit., above in this section); see also Gadula-Gacek, E., Tajstra, M., and Gasior, M. 2019. “Electrical Storm-Still an Extremely Poor Prognosis. Do These Acute States of Life-threatening Arrhythmias Require a Multidirectional Approach from the Start?,” Postepy w Kardiologii Interwencyjnej [Advances in Interventional Cardiology] (Poznań, Poland) 15(1): 1-12; Keene. D., Shun-Shin, M. J., Arnold, A. D., Howard, J. P., Lefroy, D., and 11 others 2019. “Quantification of Electromechanical Coupling to Prevent Inappropriate Implantable Cardioverter-defibrillator Shocks,” Journal of the American College of Cardiology. Clinical Electrophysiology 5(6): 705-715; Daubert, J. P., Zareba, W., Cannom, D. S., McNitt. S., Rosero, S. Z., and 9 others 2008. “Inappropriate Implantable Cardioverter-defibrillator Shocks in MADIT [Multicenter Automatic Defibrillator Implantation Trial] II: Frequency, Mechanisms, Predictors, and Survival Impact” Journal of the American College of Cardiology 51(14): 1357-1365).

Using this approach, either heart alone might present signs of failure with reduced ventricular absolute stroke volume. If vascular resistance had been a problem for the native heart and a heart-lung transplant was not possible or contraindicated, then a second heart in tandem may allow the pressure of ejection needed to be achieved. When indicated, the likelihood of ventricular fibrillation is reduced by the periodic directly pipeline targeted release of one or more direct contact, or topical, antiarrhythmic drugs (Chatzidou. S., Georgiopoulos. G., and Kontogiannis, C. 2018. “Implications of Aging in the Treatment of Complex Arrhythmias,”Aging (Albany, New York) 10(11): 3048-3049; van Bavel, J. J. A., Vos, M. A., and van der Heyden, M, A, G. 2018. “Cardiac Arrhythmias and Antiarrhythmic Drugs: An Autophagic Perspective,” Online, Frontiers in Physiology 9:127; Ayan, M., Pothineni, N. V., Siraj. A., and Mehta, J. L. 2016. “Cardiac Drug Therapy—Considerations in the Elderly,” Journal of Geriatric Cardiology 13(12): 992-997; Deneer, V, H. and van Hemel, N. M. 2011. “Is Antiarrhythmic Treatment in the Elderly Different? A Review of the Specific Changes,” Drugs and Aging 28(8): 617-633; Singh, S. N., Patrick, J., and Patrick, J. 2004. “Antiarrhythmic Drugs,” Current Treatment Options in Cardiovascular Medicine 6(5): 357-364; Mason, J. W. 1993. “A Comparison of Seven Antiarrhythmic Drugs in Patients with Ventricular Tachyarrhythmias. Electrophysiologic Study versus Electrocardiogramonitoring Investigators,” New England Journal of Medicine 329(7): 452-458; Roy, D. 1986. “Antiarrhythmic Drugs,” Canadian Anaesthetists Society Journal 33(3 Part 2): S65-S67; Kelliher, G. J., Kowey, P., Engel. T., and Wetstein, L. 1985. “Clinical Pharmacology of Antiarrhythmic Agents,” Cardiovascular Clinics 16(1): 287-305; Kienzle, M. G., Williams, P. D., Zygmont, D., Doherty, J. U., and Josephson, M, E. 1984. “Antiarrhythmic Drug Therapy for Sustained Ventricular Tachycardia,” Heart and Lung: The Journal of Critical Care 13(6): 614-622) not dependent upon processing by the liver, each according to its time to onset, directly into the venae cavae of either or both hearts through accessory channels in the diversion servovalves.

If release into the circulation of the antiarrhythmic would pose the risk of adverse side effects (see, for example, Pandya, B., Spagnola, J., Sheikh, A., Karam, B., Anugu, V. R., and 4 others 2016. “Anti-arrhythmic Medications Increase Non-cardiac Mortality-A Meta-analysis of Randomized Control Trials,” Journal of Arrhythmia 32(3): 204-211) and a reversal agent is available, the reversal agent is released into the aorta through accessory channels in the diversion servovalves for immediate delivery into the end-arterial coronaries (see, for example, Lee, H. C., Matsuda, J. J., Reynertson, S. I., Martins, J. B., and Shibata, E, F. 1993. “Reversal of Lidocaine Effects on Sodium Currents by Isoproterenol in Rabbit Hearts and Heart Cells,” Journal of Clinical Investigation 91(2): 693-701: Calkins, H., Sousa, J., e1-Atassi, R., Schmaltz, S., Kadish, A., and Morady, F. 1992. “Reversal of Antiarrhythmic Drug Effects by Epinephrine: Quinidine versus Amiodarone,” Journal of the American College of Cardiology 19(2): 347-352; DiMarco, J. P. 1992. “Epinephrine Infusions during Antiarrhythmic Drug Evaluations: Toward a Better Understanding of Drug Activity,” Journal of the American College of Cardiology 19(2): 353-354; Steinbeck, G., Andresen, D., Bach, P., Haberl, R., Oeff, M., Hoffmann, E., and von Leitner. E, R. 1992. “A Comparison of Electrophysiologically Guided Antiarrhythmic Drug Therapy with Beta-blocker Therapy in Patients with Symptomatic, Sustained Ventricular Tachyarrhythmias,” New England Journal of Medicine 327(14): 987-992; Morady, F., Kou, W. H., Kadish, A. H., Nelson, S. D., Toivonen, L. K., and 3 others 1988. “Antagonism of Quinidine's Electrophysiologic Effects by Epinephrine in Patients with Ventricular Tachycardia,” Journal of the American College of Cardiology 12(2): 388-394). Moving through the Singh-Vaughan Williams classification of antiarrhythmic agents, and omitting those primarily directed to atrial dysrhythmia, those pertinent include Class Ia, quinidine, Class Ib, tocainide, Class Ic, flecainide, Class II, carvedilol, Class III, amiodarone, Class IV, diltiazem, and Class V, magnesium sulfate.

If no reversal agent is available, the drug is formulated as a nanoparticulate bonded to a nanoparticulate superparamagnetic carrier for extraction from the blood ejected either by diversion valve-jackets with internal magnets or basic side-entry jackets incorporating a concentric magnet magnetized normal or perpendicularly to the luminal axis of the substrate vessel. When the cardioverter defibrillator as primary level sensor feeding the implanted master control microprocessor signals a menacing ventricular fibrillation, the microprocessor initiates an algorithm to variously adjust the apportionment of the servovalves at prescribed frequencies to favor recovery of the affected heart of hearts by altering the apportionment between the two of the venous retum volume respective of each likely to alleviate the fibrillation.

Using this approach, two marginal hearts that would otherwise have been rejected for transplantation do not go to waste, can be synergistically coordinated to approximate the function of an unimpaired native heart, and so can sustain circulatory function with greater durability than could an assist device were one available. The ultimate objects in this is not only to afford an additional option for ameliorating the condition of the individual patient, but alleviate the shortage in available hearts. The assist heart can, and unless and until a good heart is found, be left in place indefinitely. Double heart transplantation metered or otherwise absent from the literature, it cannot be stated with authority that an assist heart would allow the primary or native heart to at least partially recover function; however the segregable targetability of the assist heart makes this substantially more probable than the assist heart in a conventional heterotopic transplant.

Such a presupposition is, however, compelling, and because the function of each heart can be separately affected, the approach overcomes the deterrent to combine hearts considerably mismatched in size or ventricular absolute stroke volume. It is known from conventional orthotopic transplantation that over- and undersized hearts both gradually adapt to their new situation, an oversized heart gradually relaxing it force of output, and an undersized heart gradually increasing in left ventricular myocardial strength. With a significantly oversized heart, the serious sequelae due to hypertension that ensue following the operation, to include coma and convulsions, have proven controllable (references provided below in this section).

From this, it must be assumed that two intact hearts would gradually adapt to one another. Because the discrepancy in size of the hearts in a conventional heterotopic transplantation is limited, the potential for either heart to adapt to the other had it been separate is likewise limited. Of the causes cited for post-transplant ventricular malfunction-anesthetic, drug-induced, and/or surgical trauma related none pertain to switch transplantation. Counterintuitively, with an implanted automatic response system as delineated in U.S. Pat. No. 11,759,186, entitled Ductus Side-entry Jackets and Prosthetic Disorder Response Systems, to control the excessive ventricular absolute stroke volume or off-timing of an oversized heart, temporary post-transplantation impairment in left ventricle function might even prove beneficial.

That in a conventional heterotopic heat transplant, the difference in size between donor and native hearts is limited is in itself an additional deterrent, which along with more stringent selection criteria, eliminate hearts in a marginal condition, adding considerably to the shortage of hearts available for transplant (Laredo, M., Varnous, S., Balagny, P., Leprince, P., Hidden-Lucet, F., and Duthoit, G. 2017. “Successful Catheter Ablation of Recipient Left Ventricular Tachycardia 26 Years after Heterotopic Heart Transplantation,” Heart Rhythm Case Reports 3(3): 159-163; Hassoulas, J. 2012. “Transplantation of the Heart: An Overview of 40 Years' Clinical and Research Experience at Groote Schuur Hospital and the University of Cape Town: Part I. Surgical Experience and Clinical Studies,” South African Medical Journal 102(6 Part 2): 350-352; Chu, S. H., Chiu, K. M., Lin, T. Y., Chu, T. W., Yeih, D. F., and Li, A, H. 2007, Op cit. above in this section; see also Holinski, S., Hausdorf. G., and Konertz, W. 2016. “From Baby to Man with a Piggyback Heart: Long-term Success of Heterotopic Heart transplantation,” European Journal of Cardiothoracic Surgery 49(1): 348-349; Elefteriades, J., Lovoulos, C., Edwards, R., Tittle, S., Riley, T., and 3 others 2003. “Novel Technique for Isolated Accessory Right Heart Transplantation for Congenital Heart Disease,” Journal of Thoracic and Cardiovascular Surgery 125(6): 1283-1290; Inoue. T., Otaki, M., Kaneda. T., and Oku, H. 2001. “Experimental Heterotopic Heart Transplantation without Cardiopulmonary Bypass: Auxiliary Support for the Recipient Heart,” Journal of Heart and Lung Transplantation (New York, New York) 20(3): 364-371; Hassoulas, J. and Barnard, C. N. 1984. “Heterotopic Cardiac Transplantation. A 7-year Experience at Groote Schuur Hospital, Cape Town,” South African Medical Journal 65(17): 675-682; Barnard, C. N., Barnard, M. S., Cooper, D. K. C., Curchio, C. A., Hassoulas, J., Novitsky, D., and Wolpowitz, A. 1981. “The Present Status of Heterotopic Cardiac Transplantation,” Journal of Thoracic and Cardiovascular Surgery 81(3): 433-439) or if having been intended ab initio as a bridge to recovery of the native heart, the size-mismatched heart is removed when it is no longer needed. It can also be removed having allowed the native heart time to recover but then itself having become infected or otherwise impaired (Tsang, V., Yacoub, M., Sridharan, S., Burch, M., Radley-Smith, R., and 3 others 2009. “Late Donor Cardiectomy after Paediatric Heterotopic Cardiac Transplantation,” Lancet 374(9687): 387-392).

Other means described herein facilitate correction of a noncomplex transposition of the great arteries using synthetic materials and relying upon transluminal or transcatheteric techniques to repair associated intracardiac defects such as septal and valvular and either conventional repair or means addressed below in section (c). Aortic Repair, to Include Coarctation and Aneurysm, and Targeted Drug Delivery. Also addressed are aortic coarctation and aneurysm (see, for example, Lacour-Gayet, F. 2012. “Complexity Stratification of the Arterial Switch Operation: A Second Learning Curve,” Cardiology in the Young (Cambridge, England) 22(6): 739-744; Talwar, S., Shivaprasad, M. B., Kothari, S. S., and Choudhary, S. K. 2009. “A Novel Conduit-lengthening Technique to Facilitate the Arterial Switch Operation in an Infant with a Problematic Combination of Coronary Anomalies,” Texas Heart Institute Journal 36(3): 234-237; Suzuki, T. 2009. “Modification of the Arterial Switch Operation for Transposition of the Great Arteries with Complex Coronary Artery Patterns,” General Thoracic and Cardiovascular Surgery 57(6): 281-292; Karl. T. R., Cochrane, A., and Brizard, C. P. 1997. “Arterial Switch Operation. Surgical Solutions to Complex Problems,” Texas Heart Institute Journal 24(4): 322-333; additional references cited below in sections 2b(2)(a), entitled Transectionless and ischemia-free Correction of Complete or Dextro-transposition of the Great Arteries, and 2b(2)(b), entitled Bidirectional Valves for Perinatal Extracardiac Correction of Complete or Dextro-transposition of the Great Arteries and Reciprocal Cross-circulation).

Similar means allow a total or partial exchange of blood flow between or among vessels to adjust the blood pressure in each, thereby ameliorating pulmonary or portal hypertension, for example, or the adverse effects of placing a heart in a neonate or infant, too large to adapt to the reduced load in a reasonable time. Applicable to any type ductus, valve and servovalve flow diversion jackets can also serve as a means for the permanent or night-time-only diversion of urine. The advantages in the urinary tract to be gained through the use of synthetic materials introduced with minimal trauma are considerable.

Urinary diversion by means other than reconstructive eliminates the need to harvest native tissue, typically gut, which is ill-adapted and susceptible to develop cancer when used to pass urine. Harvesting ill-adapted tissue involves a preliminary procedure with its own potential risks and complications to include significant injury to the gut, and calls for much additional dissection, increased procedural duration, and when necessary, general anesthesia time.

In applying diversion jackets to transplantation of the heart, for example, the abrupt switching of circulation through the recipient from the native to the donor heart without the need for ventilator or cardiopulmonary bypass machine support eliminates damage to the erythrocytes, generates no microemboli or debris, avoids the risk of post perfusion syndrome, or ‘pump headedness,’ with cognitive impairment, and there is no breakdown or spalling of tubing with the release of debris into the blood as has been experienced (see, for example, Liu, Y., Faria, M., and Leonard, E. 2016. “Spallation of Small Particles from Peristaltic Pump Tube Segments,”Artificial Organs October 13; Spiwak, A. J., Horbal, A., Leatherbury, R., and Hansford, D. J. 2008. “Extracorporeal Tubing in the Roller Pump Raceway: Physical Changes and Particulate Generation,” Journal of Extra-corporeal Technology 40(3): 188-192). No less important, the pressure drop associated with cross-clamping using the conventional technique is avoided.

Such valves incorporate an intravascular, or endoluminal, diversion chute which allows diverting all or a continuously variable fraction of the blood supply to and the drainage from the native to the graft organ before the graft organ is harvested, thus eliminating the ischemia-reperfusion injury that fundamentally degrades the graft, materially reducing the odds for success in any solid organ transplantation. This capability instituted, medical centers should reserve an area—a donor, not just an organ, maintenance repository—for deceased organ donors on life support (Hanif, Z., Sufiyan, N., Patel, M., and Akhtar, M. Z. 2018. “Role of Biobanks in Transplantation,” Annals of Medicine and Surgery (London, England) 28:30-33; Wheelock, A. M., Paulson, L., and Litton, J, E. with 8 collaborators 2015. “The EuPA [European Proteomics Association] Biobank Initiative: Meeting the Future Challenges of Biobanking in Proteomics and Systems Medicine,” Journal of Proteomics 127 (Part B): 414-416; Somiari, S, B. and Somiari, R. I. 2015. “The Future of Biobanking: A Conceptual Look at How Biobanks Can Respond to the Growing Human Biospecimen Needs of Researchers,” Advances in Experimental Medicine and Biology 864:11-27).

Whereas a functioning brainstem may sustain body temperature, endocrine, circulatory, pulmonary, digestive, excretive, metabolic, and other bodily functions, in a corpse, these must all be sustained in a coordinated, homeostatic manner artificially (see, for example, Park, J., Yang, N. R., Lee, Y. J., and Hong, K. S. 2018. “A Single-center Experience with an Intensivist-led Brain-dead Donor Management Program,” Annals of Transplantation 23:828-835; Nakagawa. T. A., Shemie, S. D., Dryden-Palmer, K., Parshuram, C. S., and Brierley, J. 2018. “Organ Donation following Neurologic and Circulatory Determination of Death,” Pediatric Critical Care Medicine 19(8S Supplement 2): S26-S32; Sanchez-Vallejo, A., Gomez-Salgado, J., Fernandez-Martinez, M. N., and Fernandez-Garcia, D. 2018. “Examination of the Brain-dead Organ Donor Management Process at a Spanish Hospital,” International Journal of Environmental Research and Public Health 15(10). pii: E2173; Souter, M. J., Eidbo, E., Findlay, J. Y., Lebovitz, D. J., Moguilevitch, M., and 6 others 2018. “Organ Donor Management: Part 1. Toward a Consensus to Guide Anesthesia Services during Donation after Brain Death,” Seminars in Cardiothoracic and Vascular Anesthesia 22(2): 211-222; Chamorro-Jambrina, C., Muñoz-Ramirez, M. R., Martinez-Melgar, J, L., and Perez- Comejo, M. S. 2017. “Organ Donor Management: Eight Common Recommendations and Actions that Deserve Reflection,” Medicina Intensiva (Madrid, Spain) 41(9): 559-568; Shetty. V, L., Mali, S. S., Shetty, S. V., and Shinde, P, D. 2017, Op cit., this section; Pandit, R. A., Zirpe, K. G., Gurav, S. K., Kulkarni, A. P., Kamath, S., and 10 others 2017. “Management of Potential Organ Donor: Indian Society of Critical Care Medicine: Position Statement:” Indian Journal of Critical Care Medicine 21(5): 303-316; Chamorro-Jambrina, C., Muñoz-Ramirez, M. R., Martinez-Melgar, J, L., and Perez-Comejo, M. S. 2017. “Organ Donor Management: Eight Common Recommendations and Actions that Deserve Reflection,” Medicina Intensiva 41(9): 559-568; Zens, T. J., Danobeitial, J. S., Chlebeck, P. J., Zitur. L. J., Odorico, S., and 8 others 2017. “Guidelines for the Management of a Brain Death Donor in the Rhesus Macaque: A Translational Transplant Model,” Online, PLoS [Public Library of Science] One 12(9): e0182552; Zhang, L., Tian, M., Sun, L.3, and Zhu, Z. 2017. “Association between Flushed Fluid Potassium Concentration and Severe Postreperfusion Syndrome in Deceased Donor Liver Transplantation,” Medical Science Monitor 23:5158-5167; Dominguez-Gil, B., Coll. E., Elizalde, J., Herrero, J. E., Pont, T., and 11 others 2017. “Expanding the Donor Pool through Intensive Care to Facilitate Organ Donation: Results of a Spanish Multicenter Study,” Transplantation 101(8): e265-e272; Ellis, M. K., Sally, M. B., and Malinoski, D. 2016. “The Development and Current Status of Intensive Care Unit Management of Prospective Organ Donors,” Indian Journal of Urology 32(3): 178-185; Kumar, L. 2016, Op cit.; Cooper, L. B., Milano, C. A., Williams, M., Swafford, W., Croezen, D., and 3 others 2016. “Thyroid Hormone Use during Cardiac Transplant Organ Procurement,” Clinical Transplantation 30(12): 1578-1583; Mundt. H. M., Yard, B. A., Kramer, B. K., Benck, U., and Schnulle, P. 2016. “Optimized Donor Management and Organ Preservation before Kidney Transplantation,” Transplant International 29(9): 974-984; Dominguez-Gil, B., Murphy, P., and Procaccio, F. 2016. “Ten Changes that Could Improve Organ Donation in the Intensive Care Unit,” Intensive Care Medicine 42(2): 264-267; Kotloff. R. M., Blosser. S., Fulda, G. J., Malinoski. D., Ahya. V. N., and 37 others with 50 collaborators 2015. “Management of the Potential Organ Donor in the ICU: Society of Critical Care Medicine/American College of Chest Physicians/Association of Organ Procurement Organizations Consensus Statement,” Critical Care Medicine 43(6): 1291-1325; Jimenez-Castro, M. B., Gracia-Sancho, J., and Peralta, C. 2015. “Brain Death and Marginal Grafts in Liver Transplantation,” Cell Death and Disease 6: e1777; Rebolledo, R. A., Van Erp, A. C., Ottens. P. J., Wiersema-Buist. J., Leuvenink, H. G., and Romanque, P. 2015. “Anti-apoptotic Effects of 3,3′,5-Triiodo-L-Thyronine in the Liver of Brain-dead Rats,” Online, PLoS [Public Library of Science] One 10(10): e0138749; Muller, E. 2015. “Management of the Potential Organ Donor in the ICU: Society of Critical Care Medicine/American College of Chest Physicians/Association of Organ Procurement Organizations Consensus Statement,” Transplantation 99(9): 1743; Anderson, T. A., Bekker, P., and Vagefi, P, A. 2015. “Anesthetic Considerations in Organ Procurement Surgery: A Narrative Review,” Canadian Journal of Anaesthesia 62(5): 529-539; Bodi, M. A., Pont, T., Sandiumenge, A., Oliver, E., Gener, J., and 7 others 2015. “Brain Death Organ Donation Potential and Life Support Therapy Limitation in Neurocritical Patients,” Medicina Intensiva 39(6): 337-344; Murthy, T. 2009. “Organ Donation: Intensive Care Issues in Managing Brain dead,” Medical Journal, Armed Forces of India 65(2): 155-160; Shah, V, R. 2008. “Aggressive Management of Multiorgan Donor,” Transplantation Proceedings 40(4): 1087-1090; Todd, P. M., Jerome, R. N., and Jarquin-Valdivia, A, A. 2007. “Organ Preservation in a Brain-dead Patient: Information Support for Neurocritical Care Protocol Development,” Journal of the Medical Library Association 95(3): 238-245; Novitzky, D., Cooper, D. K. C., Rosendale, J. D., and Kauffman, H. M. 2006. “Hormonal Therapy of the Brain-dead Organ Donor: Experimental and Clinical Studies,” Transplantation 82(11): 1396-1401; Powner, D. J. and Hemandez, M. 2005. “A Review of Thyroid Hormone Administration during Adult Donor Care,” Progress in Transplantation 15(3): 202-207; Smith, M. 2004. Physiologic Changes during Brain Stem Death-Lessons for Management of the Organ Donor,” Journal of Heart and Lung Transplantation (New York, New York) 23(9 Supplement): S217-S222).

Following preoperative low dose radiation, microchimerization in preparation for transplantation is accomplished to the extent possible through the exchange of various cell types, which may include thymocytes, hepatocytes, splenocytes, blood and/or hematopoietic stem cells, bone marrow cells, umbilical cord mesenchymal stromal, and/or liver-derived stem cells (see, for example, Sasaki, H., Oura, T., Spitzer, T. R., Chen, Y. B., Madsen, J. C., and 4 others 2018. “Preclinical and Clinical Studies for Transplant Tolerance via the Mixed Chimerism Approach,” Human Immunology (Amsterdam, North Holland, Netherlands) 79(5): 258-265; Sprangers, B., DeWolf, S., Savage, T. M., Morokata, T., Obradovic, A., and 16 others 2017. “Origin of Enriched Regulatory T Cells in Patients Receiving Combined Kidney-Bone Marrow Transplantation to Induce Transplantation Tolerance,” American Journal of Transplantation 17(8): 2020-2032; references on radiation and cell infusion are provided below in section 2b(1)(b)(b2), entitled Prenatal Compound Vascular Bypass, or Sudden Switched-organ Transplantation of the Heart). Since metered compound vascular bypass, or sudden switched-organ transplantation involves no interruption in blood flow, the process of concurrent organ transplantation and immune tolerance induction, if necessary, can proceed over a period of days.

Cells obtained from the donor and recipient are exchanged for cross-infusion or an alternative conventional form of administration. Where possible, the different type cells are co-packed. To allow such an exchange of viable cells, whenever possible, the donor on life support should not be allowed to become unfit as the source of preliminary tolerance-inducing cells. If necessary, the cells are exchanged between donor and recipient by transporting the recipient to the donor on life support for the switch transplantation. In this way, one donor can be reciprocally cross-microchimerized with multiple recipients each to receive different organs.

Maintaining the deceased donor on intensive somatic support to include a normal sustained body temperature and endocrine function without interruption in perfusion not only lessens the immunological and hematological impact of brain death followed by the additional trauma of excision with no blood flow, best preserving the graft organ itself, but makes it possible for the dead donor as well as the recipient to participate in cross-microchimerization whereby the body of both donor and recipient can be reciprocally cross-microchimerized by cells taken from the recipient as well as the recipient microchimerized with living cells taken from the donor. The graft organ thus less recoils upon transfer into the circulatory system of the recipient, and the immune system of the recipient less recoils to the presence of the graft organ in his body.

When an interval elapses from this preparatory tolerance induction, the blood exchanged during this reciprocal cross-circulation, or cross-transfusion, is not only better matched but more able to prevent rejection. Preliminary reciprocal cross-transfusion in itself can impart a measure of graft acceptance (Bloch, E. M., Jackman, R. P., Lee, T. H., and Busch, M. P. 2013, Op cit., section 2a; other references above in this section and others below) but will not in itself accomplish mutual cross-microchimerization. Rather, this is accomplished by immunological preconditioning by preprocedurally cross-transfusing products specified above obtained from either.

Preliminary matching of donor and recipient must rule out a significant load or preformed antibodies in the recipient as would immediately precipitate a hyperacute rejection. If not excessive, a preformed load of antibodies may be reduced through plasmapheresis (plasma exchange, immunoadsorption) concomitant with the impairment thereof with the aid of immunoglobulins and rituximab combination therapy, for example, (see, for example, Bohmig, G. A., Eskandary, F., Doberer, K., and Halloran, P. 2019. “The Therapeutic Challenge of Late Antibody-mediated Kidney Allograft Rejection,” Transplant International 32(8): 775-788; Salvadori, M. and Tsalouchos, A. 2019. “Therapeutic Apheresis in Kidney Transplantation: An Updated Review,” Online, World Journal of Transplantation (Pleasanton, California) 9(6): 103-122; Mella, A., Gallo, E., Messina, M., Caorsi, C., Amoroso, A., and 6 others 2018. “Treatment with Plasmapheresis, Immunoglobulins, and Rituximab for Chronic-Active Antibody-mediated Rejection in Kidney Transplantation: Clinical, Immunological, and Pathological Results,” Online, World Journal of Transplantation (Pleasanton, California) 8(5): 178-187; Mulley, W. R., Huang, L, L., Ramessur Chandran, S.2, Longano, A., Amos, L, A. R., and 3 others 2017. “Long-term Graft Survival in Patients with Chronic Antibody-mediated Rejection with Persistent Peritubular Capillaritis Treated with Intravenous Immunoglobulin and Rituximab,” Clinical Transplantation (Copenhagen, Denmark) 31(9).

It warrants emphasis that these findings have not only been disputed, but the claim made that treatment thus induces a significant increase in severe infectious complications requiring hospitalization (Piñeiro, G. J. De Sousa-Amorim, E., Sole, M., Rios, J., Lozano, M., and 10 others 2018. “Rituximab, Plasma Exchange and Immunoglobulins: An Ineffective Treatment for Chronic Active Antibody-mediated Rejection,” Online, BioMed Central Nephrology (London, England) 19:261). If confirmed, a significant load of preformed antibodies presages hyperacute rejection regardless of the surgical technique used to transplant the graft organ.

Closely related to maintenance in general is the prevention or curing of disease of a prospective donor. A corpse on life support can contract an infectious disease, and with skilled immunological maintenance, be cured of most. Communicable or not, prospective donors must have been cured of disease before admission into a deceased donor repository. While incurable communicable disease must disqualify a potential donor from admittance, curable disease would best as well but need not.

The circulation sustained, the efficacy of anti-infectious drugs, such as antibiotics, viricides, and antimycotics, or fungicides, or example, should be largely if imperfectly, preserved. Affording the prospect for recovery after death, the establishment of repositories for the maintenance of prospective organ donors alone increases organ availability, the sustainment of normal circulation, hence, organ perfusion, in itself facilitates antimicrobial uptake with improvement in the ability to cure disease past death.

Rather than improving the viability of organs taken from a prospective organ donor brain-dead gravida while still on life support through continued intensive care and by sustaining if not strengthening her immune system in order to protect her organs from or cure disease prior to harvesting and donation, the literature is oriented toward delivery and conventional organ harvesting without this precaution (see, for example, Holliday, S. and Magnuson-Woodward, B. 2017. “Somatic Support following Cardiac Arrest for 90 Days Leading to a healthy Baby Boy: A Case Report,” Heart and Lung 46(5): 397-400; Gopčević. A., Rode, B., Vučić, M., Horvat. A., Siranovic, M., and 3 others 2017. “Ethical and Medical Management of a Pregnant Woman with Brain Stem Death Resulting in Delivery of a Healthy Child and Organ Donation,” International Journal of Obstetric Anesthesia 32:82-86; Wawrzyniak, J. 2015. “Continuation of Pregnancy in a Woman with Critical Brain Injury,” Anesthesiology Intensive Therapy 47(1): 40-44; Sharma, N. S., Wille, K. M., Bellot, S. C., and Diaz-Guzman, E. 2015. “Modern Use of Extracorporeal Life Support in Pregnancy and Postpartum,” American Society for Artificial Internal Organs Journal 61(1): 110-114; Kinoshita, Y., Kamohara, H., Kotera, A., Sagishima, K., Tashiro, T., and Niimori, D. 2014. “Healthy Baby Delivered Vaginally from a Brain-dead Mother,” Acute Medicine and Surgery 2(3): 211-213; Said, A., Amer, A. J., Masood, U. R., Dirar, A., and Faris, C. 2013. “A Brain-dead Pregnant Woman with Prolonged Somatic Support and Successful Neonatal Outcome: A Grand Rounds Case with a Detailed Review of Literature and Ethical Considerations,” International Journal of Critical Illness and Injury Science 3(3): 220-224; Woderska, A., Jasinski, M. Arszynska-Lopatka, D. Slupski. M., Janiszewska. T., and Wlodarczyk, Z. 2012. “A Brain-dead Woman, Giving Birth in the 23rd Week of Pregnancy, Used as an Organ Donor: A Case Report,” Annals of Transplantation 17(1): 113-116; Esmaeilzadeh, M., Dictus, C., Kayvanpour, E., Sedaghat-Hamedani, F., Eichbaum, M., and 8 others 2010. “One Life Ends, Another Begins: Management of a Brain-dead Pregnant Mother-A Systematic Review,” Online, BioMed Central Medicine 8:74; Mejia. R., Badariotti, G., De Diego, B., Ridruejo, O., and O'Flaherty, E. 2008. “Brain Death in a Pregnant Woman and Fetus Survival,” (in Spanish with English abstract at Pubmed), Medicina (Buenos Aires, Argentina) 68(6): 447-452; Souza, J. P., Oliveira-Neto, A., Surita, F. G., Cecatti, J. G., Amaral, E., Pinto, e., and Silva, J. L. 2006. “The Prolongation of Somatic Support in a Pregnant Woman with Brain- death: A Case Report,” Reproductive Health 3:3; Farragher, R, A. and Laffey, J, G. 2005. “Maternal Brain Death and Somatic Support,” Neurocritical Care 32): 99-106; Mallampalli, A. and Guy, E. 2005. “Cardiac Arrest in Pregnancy and Somatic Support after Brain Death,” Critical Care Medicine 33(10 Supplement): S325-S331).

Once cured, transplantation without interruption in normal perfusion means that organs are critically superior to organs preserved over an ischemic period. As a result, the larger pool of available organs established comprises grafts of critically improved survivability. The potential donor will likely require the administration of drugs, and in some cases, a deteriorating organ that would affect the others must be transplanted into the deceased. To accomplish this level of support requires the constant vigilance of highly specialized medical attendants with an array of diagnostic and therapeutic apparatus. For this reason, prospective organ recipients are brought to the site rather than the reverse.

It is central to the establishment of such an initiative that the effort and investment in preservation not be immediately undone due to the lack of a means for transplanting organs so that these are not damaged when harvested. The initiative is justified by vascular valves and servovalves, which eliminate ischemia in organ transplantation. Vascular choke servovalves (vascular servochokes, vascular servochoke valves, vascular servo chokevalves), are not electronic sphincters or servo chokevalves that compress or constrict the substate ductus as described and illustrated in U.S. Pat. No. 11,759,186, entitled Ductus Side-entry Jackets and Prosthetic Disorder Response Systems, but rather valves of the same kind as those containing a diversion chute where the latter is instead an adjustable obturator which can be adjusted in the extent to which it extends into the native lumen.

It is the same as a diversion servovalve but the obstructive plate or tang extended into or retracted from the lumen is planar rather than curved. The ductus side-entry jackets, side-entry intravascular diversion servovalve-jackets, extravascular and intravascular choke servovalve-jackets, described all incorporate features to protect the substrate ductus and include at least one accessory channel, or sideline, to allow the direct targeting of drugs through the device and into the substrate ductus, both critical in protecting the substrate ductus from atheromatous degradation, or atherodegradation.

The side-entry diversion jackets and valve-jackets to be described allow the creation of a dependable on-off switchable diversion path or takeoff from a native lumen and through a tube without anastomosis. Using side-entry devices, conventional anastomosis can be avoided, so that adverse tissue reactions due to direct contact between suture and the native tissue are eliminated. The same applies to catheters made of any other polymeric material when sutured in direct end-to-end or end to side anastomotic contact with a native ductus of lesser caliber rather than connected to the ductus by means of a side-entry jacket.

Irritation and/or the complete enclosure at the adventitial-foam interface, well established to promote atherosclerotic degeneration in the intima, is averted by perforations at numerous points about the outer shell of all jackets and valves which serve to expose enough of the adventitia to the surrounding environment to the body cavity. As evident in FIG. 1, in a basic side-entry jacket, water jacket/accessory channel 7 gives direct access into the jacket foam lining of counterirritant agents where the use of an open cell foam allows permeation to wet the interface.

Similarly, in diversion jackets and valves, potential complications that might be instigated at the adventitia-foam interface, primarily the inducement of atheromatous degeneration due to constriction of the adventitial vessels and nervelets, or vasa vasorum and nervorum, and continuous enclosure are averted by perforations, part number 40 in FIGS. 1, 2, 5, 7, and 8. In patients atypically sensitive to enclosure of the adventitia, a separate accessory channel positioned to wet the interface with an anti-inflammatory, for example, such as shown in FIGS. 5, 7, 8, 10A, 14, 22A, and 22B is added. With any jacket, diversion jacket, or valve, the adventitial surface can be made directly accessible to drug delivery by positioning a basic side-entry jacket with accessory channel connected to an implanted drug reservoir upstream. The upstream jacket releases the drug onto the surface of the ductus to run along the adventitia to the downstream jacket or valve.

Other approaches include passing a tiny capillary tube-gauged accessory channel-connected upstream “garden soaker hose” from an upstream jacket or through the target jacket or valve itself through the interface and the use of biomaterials which incorporate anti-inflammatory nanoparticulates, for example, and that simplest and most direct-coating the adventitia with an anti-inflammatory such as a readily absorbed preparation of triamcinolone, for example, as will not block the perforations more than momentarily, before placing the jacket or valve. While a temporary measure, this protects the substrate ductus over the period following placement when the provocation of an adverse reaction is most likely.

The foreign body reaction to the anchoring needles described and illustrated in U.S. Pat. No. 11,013,858, entitled Nonjacketing Side-entry Connectors and Prosthetic Disorder Response Systems, seeks to isolate the needles through envelopment within a fibrous capsule. From the standpoint of alleviating any discomfort, this is beneficial; however, just as it interferes with the access of implanted sensors to the tissue to be monitored, anchoring needles which are hollow to allow injection will be obstructed from doing so. Substances to suppress adverse tissue reactions can be 1. Integrated into the implant material. 2. Applied thereto as an outer coating, or 3. Released as a fluid from an accessory channel.

Whether incorporated into ductus side-entry jackets, vascular valves, or servovalves, or implanted separately and supported by nonjacketing side-entry connectors, sensors are provided with a clear ‘view’ of the targeted tissue through the release of dexamethasone and/or other substances through an integral or jacket supported service or accessory channel.

Various substances for suppressing a foreign body reaction, inflammation, and fibrous encapsulation as interferes with providing sensors a clear ‘view’ already exist and can be released either at the adventitial-foam interface or if mounted to the underside of a nonjacketing side-entry connector, through the injectable anchoring needles of the nonjacketing connector (see, for example, Gu, B., Papadimitrakopoulos, F., and Burgess, D. J. 2018. “PLGA [poly(lactic-co-glycolic acid)] Microsphere/PVA [polyvinyl alcohol] Hydrogel Coatings Suppress the Foreign Body Reaction for 6 Months,” Journal of Controlled Release 289:35-43; Rong. G., Corrie, S. R., and Clark, H, A. 2017. “In Vivo Biosensing: Progress and Perspectives,” American Chemical Society Sensors 2(3): 327-338; Soto, R. J., Hall, J. R., Brown, M. D., Taylor, J. B., and Schoenfisch, M, H. 2017. “In Vivo Chemical Sensors: Role of Biocompatibility on Performance and Utility,” Analytical Chemistry 89(1): 276-299; Kastellorizios, M., Tipnis, N., Papadimitrakopoulos, F., and Burgess, D. J. 2015. “Drug Distribution in Microspheres Enhances their Anti-inflammatory Properties in the Gottingen Minipig,” Molecular Pharmaceuties 12(9): 3332-3338; Wang, Y., Papadimitrakopoulos, F., and Burgess, D. J. 2013. “Polymeric “Smart” Coatings to Prevent Foreign Body Response to Implantable Biosensors,” Journal of Controlled Release 169(3): 341-347; Yan, Q., Major, T. C., Bartlett, R. H., and Meyerhoff, M, E. 2011. “Intravascular Glucose/Lactate Sensors Prepared with Nitric Oxide Releasing Poly(lactide-co-glycolide)-based Coatings for Enhanced Biocompatibility,” Biosensors and Bioelectronics 26(11): 4276-4282; Morais, J. M., Papadimitrakopoulos, F., and Burgess, D. J. 2010. “Biomaterials/Tissue Interactions: Possible Solutions to Overcome Foreign Body Response,” American Association of Pharmaceutical Scientists Journal 12(2): 188-196; Klueh, U., Kaur, M., Montrose, D. C., and Kreutzer, D. L. 2007. “Inflammation and Glucose Sensors: Use of Dexamethasone to Extend Glucose Sensor Function and Life Span in Vivo,” Online, Journal of Diabetes Science and Technology (Thousand Oaks, California) 1(4): 496-504).

Another, potentially concomitant approach, is the use of materials with intrinsic resistance to adverse tissue reactions (see, for example, Christo, S. N., Diener, K. R., Bachhuka, A., Vasilev, K., and Hayball, J, D. 2015. “Innate Immunity and Biomaterials at the Nexus: Friends or Foes,” Online, BioMed Research International 2015:342304; Wang, Y., Vaddiraju, S., Gu, B., Papadimitrakopoulos, F., and Burgess, D. J. 2015. “Foreign Body Reaction to Implantable Biosensors: Effects of Tissue Trauma and Implant Size,” Journal of Diabetes Science and Technology 9(5): 966-977; Benko, A., Frączek-Szczypta, A., Menaszek, E., Wyrwa, J., Nocun, M., and Blażewicz, M. 2015. “On the Influence of Various Physicochemical Properties of the CNTs [carbon nanotubes] Based Implantable Devices on the Fibroblasts' Reaction in Vitro,” Journal of Materials Science. Materials in Medicine 26(11): 262; Nair, A., Zou, L., Bhattacharyya, D., Timmons, R. B., and Tang, L. 2008. “Species and Density of Implant Surface Chemistry Affect the Extent of Foreign Body Reactions,” Langmuir 24(5): 2015-2024; Thevenot, P., Hu, W., and Tang, L. 2008. “Surface Chemistry Influences Implant Biocompatibility,” Current Topics in Medicinal Chemistry 8(4): 270-280; Kamath, S., Bhattacharyya, D., Padukudru, C., Timmons, R. B., and Tang, L. 2008. “Surface Chemistry Influences Implant-mediated Host Tissue Responses,” Journal of Biomedical Materials Research. Part A 86(3): 617-626).

Direct synthetics-tissue contact uses materials inherently, or through surface treatment, least likely to provoke an adverse tissue reaction. For this reason and to protect the foam jacket or connector lining from hydrolytic or enzymatic breakdown, the foam—as is the entire assembly process for all types of system end connectors—is applied in a clean room with a thin vapor-deposited coating of an elastic parylene (Galeotti, F., Andicsova, A., Bertini, F., Laux, E., Hartmann, L., and 6 others 2014. “Enhanced Elasticity in Parylene Thin Films by Copolimerization Approach,” Journal of Materials Science 49(21): 7547-7555) which further protects against microbial intrusion.

Cited below, as is now conventional in the manufacture of catheters, additional protection against microbial intrusion is imparted through the use of a polymeric shell material such as plasticizer-free polyether ether ketone or nylon embedded with an antimicrobial. The foam lining can itself be lined with nanometrically thin film of vapor deposited elastic parylene embedded with antibiotic nanoparticles. To prevent atherosclerotic degeneration, jackets and valve-jackets for use along arteries must include through-wall apertures of which the inner walls of the perforations must likewise be coated thus.

An adverse tissue reaction can be ameliorated through similarly embedding a steroid such as triamcinolone, dexamethasone, acetylcholine, or a nonsteroidal anti-inflammatory such as curcumin, suitable antimicrobials numerous, and where an especially refractory adverse reaction might arise, one or more capillary-gauged, soft polymeric walled tubes with perforations along the sides much as a ‘soaker hose,’ or side dripping line, connected to an accessory channel can be used to deliver remedial agents directly into the adventitial-foam interface.

The direct pipeline-targeting of drugs to nidi or lesions means that in contradistinction to systemic delivery, doses are at once fully utilized, do not affect nontargeted tissue, and can therefore be increased, possibly with improved efficacy or indications as to new applications (see, for example, Kern, D. M., Cepeda, M. S., Lovestone, S., and Seabrook, G, R. 2019. “Aiding the Discovery of New Treatments for Dementia by Uncovering Unknown Benefits of Existing Medications,” Alzheimer's and Dementia (New York, New York) 5:862-870).

Specific anti-inflammatoires suitable for treating an adverse tissue reaction topically, or by direct application, here, by direct piping to the treatment site or its blood supply, include dexamethasone, prednisone, prednisolone, phosphorylcholine, cortisone, and possibly, curcumin (diferuloylmethane), reported to possess antimicrobial, antioxidant, anti-neoplastic, and other useful, as well as anti-inflammatory properties (see, for example, Adahoun, M. A., Al-Akhras, M. H., Jaafar, M. S., and Bououdina, M. 2017. “Enhanced Anti-cancer and Antimicrobial Activities of Curcumin Nanoparticles,” Artificial Cells, Nanomedicine, and Biotechnology 45(1): 98-107; Hewlings, S. J. and Kalman, D. S. 2017. “Curcumin: A Review of Its' Effects on Human Health,” Foods (Basel, Switzerland) 6(10). pii: E92; Vaughn, A. R., Branum, A., and Sivamani, R. K. 2016. “Effects of Turmeric (Curcuma longa) on Skin Health: A Systematic Review of the Clinical Evidence,” Phytotherapy Research 30(8): 1243-1264; Shome, S., Talukdar, A. D., Choudhury, M. D., Bhattacharya, M. K., and Upadhyaya, H. 2016. “Curcumin as Potential Therapeutic Natural Product: A Nanobiotechnological Perspective,” Journal of Pharmacy and Pharmacology 68(12): 1481-1500; Kastellorizios, M., Tipnis, N., and Burgess, D. J. 2015, Op cit.; Reeves, A., Vinogradov, S. V., Morrissey, P., Chernin, M., and Ahmed, M. M. 2015. “Curcumin-encapsulating Nanogels as an Effective Anticancer Formulation for Intracellular Uptake,” Molecular and Cellular Pharmacology 7(3): 25-40; Khosropanah, M. H., Dinarvand, A., Nezhadhosseini, A., Haghighi, A., Hashemi, S., and 5 others 2016. “Analysis of the Antiproliferative Effects of Curcumin and Nanocurcumin in MDA-MB231 as a Breast Cancer Cell Line,” Iranian Journal of Pharmaceutical Research 15(1): 231-239; Alok, A., Singh, I. D., Mendonça, L. M., Machado Cda, S., Teixeira, C. C., Freitas, L. A., Bianchi, M, L., and Antunes, L. M. 2015. “Comparative Study of Curcumin and Curcumin Formulated in a Solid Dispersion: Evaluation of Their Antigenotoxic Effects,” Genetics and Molecular Biology 38(4): 490-498; Martinez-Cifuentes, M., Weiss-Lopez, B., Santos, L. S., and Araya-Maturana, R. 2015. “Heterocyclic Curcumin Derivatives of Pharmacological Interest: Recent Progress,” Current Topics in Medicinal Chemistry 15(17): 1663-1672; Singh, S., Kishore, M., and Jha, P, C. 2015. “Curcumin-Pharmacological Actions and Its Role in Oral Submucous Fibrosis: A Review,” Journal of Clinical and Diagnostic Research 9(10): ZE01-ZE03; Moghadamtousi, S. Z., Kadir, H. A., Hassandarvish, P., Tajik, H., Abubakar, S., and Zandi, K. 2014. “A Review on Antibacterial, Antiviral, and Antifungal Activity of Curcumin,” Online, BioMed Research International 2014:186864; Afshariani, R., Farhadi, P., Ghaffarpasand, F., and Roozbeh, J. 2014. “Effectiveness of Topical Curcumin for Treatment of Mastitis in Breastfeeding Women: A Randomized, Double-blind, Placebo-controlled Clinical Trial,” Oman Medical Journal 29(5): 330-334; Prasad, S., Gupta, S. C., Tyagi, A. K., and Aggarwal, B, B. 2014. “Curcumin, a Component of Golden Spice: From Bedside to Bench and Back,” Biotechnology Advances 32(6): 1053-1064).

Intact turmeric appears to have wider application than its 2-5 percent constituent curcumin (Aggarwal, B. B., Yuan, W., Li, S., and Gupta, S, C. 2013. “Curcumin-free Turmeric Exhibits Anti-inflammatory and Anticancer Activities: Identification of Novel Components of Turmeric,” Molecular Nutrition and Food Research 57(9): 1529-1542; Gupta, S. C., Sung, B., Kim, J. H., Prasad, S., Li, S., and Aggarwal, B, B. 2013. “Multitargeting by Turmeric, the Golden Spice: From Kitchen to Clinic,” Molecular Nutrition and Food Research 57(9): 1510-1528). However, the use of curcumin as an isolate requires preliminary hypersensitivity testing (Chaudhari, S. P., Tam, A. Y., and Barr, J, A. 2015. “Curcumin: A Contact Allergen,” Journal of Clinical and Aesthetic Dermatology 8(11): 43-48; Davari, P. and Maibach, H.1. 2011. “Contact Urticaria to Cosmetic and Industrial Dyes,” Clinical and Experimental Dermatology 36(1): 1-5; Liddle, M., Hull, C., Liu, C., and Powell, D. 2006. “Contact Urticaria from Curcumin,” Dermatitis: Contact, Atopic, Occupational, Drug 17(4): 196-197). Whether intact turmeric reverses some potentially adverse effects of curcumin alone is not addressed in the literature.

Vascular valves and servovalves are ductus side-entry jackets which incorporate a diversion chute that when intromitted within the substrate lumen of a ureter allows urine to be diverted directly into a collection bag or blood to be diverted from one vessel into another. Actuated by plunger (thrust rod, slider, shaft, punching, or push-pull), solenoids, bistable valves, switchable from fully closed to fully open for use in sudden compound vascular bypass, or sudden switched-organ solid organ transplantation are not continuously variable in response to commands issued by an implanted microprocessor executing a prescription-program in response to data fed back to the microprocessor by process reaction-detecting sensors. For this reason, the valves used must be servovalves. This factor limits solenoid valves to one-for one, that is, initial solid organ replacement or later retransplantation. Double organ transplantation necessitates control over diversion valve extension after the transplantation has been completed and therefore also requires servovalves.

The major applications of vascular valves and servovalves include compound vascular bypass of native to replacement solid organs to allow transplantation without interruption in perfusion, make possible the extracardiac correction of complete or dextro-transposition of the great arteries, bypass a coarctation of the aorta, and adjust the blood pressure between or among vessels, such as to dispel pulmonary hypertension. When the volume of blood to pass calls for adjustment, for example, the valve-jackets are servovalves responsive to an implanted blood pressure sensor signal-fed controller.

For use along the urinary tract, endoluminal valves as ureteral takeoffs impart new control over the diversion of urine by ureteral takeoff into a collection bag. The valve-jackets communicate with a tiny port at the body surface with openings for the introduction of drugs directly pipeline-targeted to the treatment site or to outlet urine. The direct pipeline targeting of a drug that requires first pass metabolism in the liver and kidneys can be released into the portal vein or a tributary proximal to it or if for any reason first pass metabolism would be counterproductive the drug can be released directly into the target tissue through a nonjacketing side entry jacket as described and illustrated in U.S. Pat. No. 11,013,858, entitled Nonjacketing Side-entry Connectors and Prosthetic Disorder Response Systems.

This capability significantly increases bioavailability and potency, and substantially reduced if not eliminates exposure of nontargeted tissue to the drug. By omitting the bladder and its highly pressure sensitive trigone, ureteral takeoff eliminates urge sensation, permitting uninterrupted sleep or public performance due to the need to relieve oneself. Most drugs, and certainly more frequently used steroids and chemotherapeutics, for example, harbor potentially and seriously harmful side effects for unintended organs and tissues, and while some negligible run-off into the systemic circulation from the targeted tissue is possible, direct targeting substantially reduces if not eliminates the effect.

Another significant benefit is the relatively small dose required when a newer drug would otherwise be unaffordable. Most applications simple, in patients with complex comorbid disease, the ductus side-entry jackets, nonjacketing side-entry connectors, valve-jackets and servovalves are supported for the directly pipe-targeted release of medication or electrostimulation, for example, by an implanted automatic adaptive hierarchical ambulatory prosthetic disorder response control system. With such a system, different channels or control arms can be assigned to monitor disease processes whether interrelated, as in metabolic syndrome, or where each is more independent and respond therapeutically with medication or electrostimulation, for example, as appropriate.

A master controller microprocessor is fed physiological sensory data which passes up through a series of intermediate hierarchical level microcontrollers, each coordinating the incoming data at its respective levels. Based upon this information, the microprocessor prescription-program is able to optimize the treatment of each morbidity individually and as a component in the combination of disorders to optimize the overall homeostasis and health of the patient. All the organs and tissues of the body interdependent, the presence of multiple interdependent morbidities is usually characteristic in the elderly with heart failure, for example, where cardiorenal, cardiohepatic, and more recently having come to light, and cardio-oncology relating to cardiovascular disease caused by chemo- and other cancer therapy, cardiooncologic impairment, are interdependent.

That heart disease had been contributed to if not entirely produced iatrogenically as the result of chemotherapy- or radiation-induced cardiotoxicity, however had already become evident. Radiation-induced arterial toxicity is addressed below in section 2b(2)(g), entitled Ischemia and Hypoperfusion-free Carotid Endarterectomy Bypass and Prosthesis. The cardiotoxicity of cardiooncologic therapy joins the myriad side effects of medication systemically dispersed to reach all susceptible tissues and organs when direct pipeline targeting would not only avoid such potentially catastrophic consequences but allow the targeted dose to be increased.

Given that the automatic direct pipeline targeting of drugs to the root, or site, of disease of a malignancy by an implanted disorder control system, not all cardiopathy or cardiomyopathy coincident with malignancy can be definitively attributed to chemotoxicity or radiation. This is certainly the case where the heart disease preceded the cancer and no radiation had been used. It is becoming clear that heart disease and cancer are not unidirectional-given that both have much the same risk factors, heart disease and cancer can arise together, and it appears that the former can even induce cancer (see, for example, Bertero, E., Ameri, P., and Maack C. 2019. “Bidirectional Relationship between Cancer and Heart Failure: Old and New Issues in Cardio-oncology,” Cardiac Failure Review 5(2): 106-111; de Boer, R. A., Meijers, W. C., van der Meer, P., van Veldhuisen, D. J. 2019. “Cancer and Heart Disease: Associations and Relations,” European Journal of Heart Failure July 18; Meijers, W, C. and de Boer, R, A. 2019. “Common Risk Factors for Heart Failure and Cancer,” Cardiovascular Research 115(5): 844-853; Bertero, E., Canepa, M., Maack, C., and Ameri, P. 2018. “Linking Heart Failure to Cancer,” Circulation 138(7): 735-742; Cubbon, R. M. and Lyon, A, R. 2016. “Cardio-oncology: Concepts and Practice,” Indian Heart Journal 68 (Supplement 1): S77-S85; Koene, R. J., Prizment, A. E., Blaes, A., and Konety, S, H. 2016. “Shared Risk Factors in Cardiovascular Disease and Cancer,” Circulation 133(11): 1104-1114; Lee, Y. S., Chen, Y. T., Jeng, M. J.3, Tsao, P. C., Yen, H. J., and 6 others 2015. “The Risk of Cancer in Patients with Congenital Heart Disease: A Nationwide Population-based Cohort Study in Taiwan,” Online, PLoS [Public Library of Science] One 10(2): e0116844).

Magnetic means for limiting the drug to the target are addressed in U.S. Pat. No. 11,389,171 entitled Integrated System for the Infixion and Retrieval of Implants. Essentially, the drug is bound to a superparamagnetic nano- or microparticle carrier to be magnetically drawn radially outward. If the substrate is an artery and the drug is to be delivered into the intima or further abaxially, the surrounding side-entry jacket incorporates a neodymium layer magnetized perpendicularly to the long axis of the underlying vessel, or in the direction of blood flow.

To draw the drug into the parenchyma, it is delivered through the blood supply, and connectors fastened about the outer fibrous layer of the substrate organ draw the drug with carrier into the parenchyma. The soluble carrier is then broken down. To remove the drug from the bloodstream chemically, a reversal agent is released through a basic side-entry jacket, for example, as described in U.S. Pat. No. 11,759,186, entitled Ductus Side-entry Jackets and Prosthetic Disorder Response Systems, and summarized herein in sections 2a, entitled Background of the Invention, General; 2b, entitled Vascular Valves and Servovalves in the Diversion of Blood Flow, 2b(2), entitled The Intracorporeal Diversion of Blood Flow; as well as other sections herein.

Two methods for preventing a targeted drug that would be harmful to nontargeted tissue from continued movement past the target are magnetic and chemical. Where the agent to be interdicted is a radionuclide or other radioactive substance, the delivery pathway is radiation shielded, and to achieve its complete removal, both methods can be applied. If the drug is chemotherapeutic directed at a solid tumor and the risk of metastasis is judged significant, a background systemic dose minimized to suppress metastasis is provided. Shielding can be used to isolate chemo- from radiation therapeutics as well as to protect radiation hypersensitive tissue such as that of the gonads from radiation injury. An unwanted ionizing residue can be trapped inside a shielded and magnetized impasse-jacket.

The importance of confining potentially injurious drugs to the tissue intended cannot be overemphasized; solid tumor chemotherapeutics in particular demand stringent targeting with intentional systemic dosing according to the ability of the malignancy to spread. It was primarily for this purpose that side-entry jackets, connectors, and valves were devised—a permanently implanted automatic response system that targets drugs to diseased or dysfunctional tissue is dependent upon leakless and nonfouling connecting and flow control devices (see, for example, Sanchez-Cano, C. and Carril, M. 2020. “Recent Developments in the Design of Non-biofouling Coatings for Nanoparticles and Surfaces,” Online, MDPI [Multidisciplinary Digital Publishing Institute] International Journal of Molecular Sciences (Basel, Switzerland) 21(3): 1007; Wang. B., Ye, Z., Tang. Y., Han, Y., Lin, Q., and 3 others 2016. “Fabrication of Nonfouling, Bactericidal, and Bacteria Corpse Release Multifunctional Surface through Surface-initiated RAFT [reversible addition-fragmentation chain transfer] Polymerization,” Online, International Journal of Nanomedicine (Auckland, New Zealand) 12:111-125).

Antileukemic myelosuppressives such as fludarabine disrupt hematopoiesis to adversely affect the production of all, not just leukemic blood cells, thus inherently defying positionally focused targeting as applies to spatially defined or delineated tumors. To reach all hematopoietic tissue, the drug must be administered systemically. Such targeting must be chemically, molecularly targeted, rather than positionally and mechanically based. For fludarabine and other myelosuppressives to be rendered harmless to other cell lineages, new drugs selective for leukemic cells of leukemic cell receptors must be developed, or if the use of fludarabine is to continue, then collateral medication to protect nontargeted cell lines or lineages by bonding to their receptors, for example, must be formulated (see, for example, Araujo, N., Hebbar, N., and Rangnekar. V. M. 2018. “GRP78 [binding immunoglobulin protein (BiP) heat shock 70 kDa protein 5 (HSPA5), (Byun1)] is a Targetable Receptor on Cancer and Stromal Cells,” Online, EBioMedicine 33:2-3, at https://www.nebi.nlm.nih.gov/pmc/articles/PMC6085536/pdf/main.pdf; Paiva, R. A., Ramos, C. V., and Martins, V, C. 2018. “Thymus Autonomy as a Prelude to Leukemia,” Federation of European Biochemical Societies Journal 285(24): 4565-4574; Staquicini, D. I., D'Angelo, S., Ferrara, F., Karjalainen, K., Sharma, G., and 15 others 2018. “Therapeutic Targeting of Membrane-associated GRP78 in Leukemia and Lymphoma: Preclinical Efficacy in Vitro and Formal Toxicity Study of BMTP-78 [bone metastasis targeting peptidomimetic] in Rodents and Primates,” Pharmacogenomics Journal 18(3): 436-443; Karjalainen, K., Jaalouk, D. E., Bueso-Ramos, C., Bover, L., Sun, Y., and 12 others 2015. “Targeting IL11 Receptor in Leukemia and Lymphoma: A Functional Ligand-directed Study and Hematopathology Analysis of Patient-derived Specimens,” Clinical Cancer Research 21(13): 3041-3051; Vhora, I., Patil, S., Bhatt, P., Gandhi, R., Baradia, D., and Misra, A. 2014. “Receptor-targeted Drug Delivery: Current Perspective and Challenges,” Therapeutic Delivery 5(9): 1007-1024; Ratnam, M., Hao. H., Zheng, X., Wang, H., Qi. H., Lee. R., and Pan, X. 2003. “Receptor Induction and Targeted Drug Delivery: A New Antileukaemia Strategy,” Expert Opinion in Biological Therapy 3(4): 563-574).

The advantage in means that allow isolation of the heart from cardiotoxic agents and radionuclide chemotherapeutics in averting adverse side effects-such as the inducement of heart disease by a chemotherapeutic as addressed below—is incontrovertible, and attests to the advantage in direct pipeline targeting of anticancer agents to the root, or site, of disease with restriction of continued passage into the circulation, as made possible by nonjacketing side-entry connectors in combination with reversal agents if available and/or magnetic extraction.

Addressed in U.S. Pat. No. 11,013,858, entitled Nonjacketing Side-entry Connectors and Prosthetic Disorder Response Systems, radiation shielded delivery allows the use of radioactive chemotherapeutic. Antineoplastic drugs, to include those antiangiogenic, chemotherapeutic, as well as steroids, for example, are invariably harmful to other tissue. Absent means for contained targeting, radioactive chemotherapeutics will continue to be precluded and unstudied.

At the same time, unless the malignancy is too close to the heart or other organ that might be affected, direct beam radiation is usually focused enough to avoid a malignancy-inducing spillover of radiation (Seraphim, A., Westwood, M., Bhuva, A. N., Crake. T., Moon, J. C., and 6 others 2019. “Advanced Imaging Modalities to Monitor for Cardiotoxicity,” Current Treatment Options in Oncology 20(9): 73; Plana, J. C., Thavendiranathan, P., Bucciarelli-Ducci, C., and Lancellotti, P. 2018. “Multi-modality Imaging in the Assessment of Cardiovascular Toxicity in the Cancer Patient,” Journal of the American College of Cardiology. Cardiovascular Imaging 11(8): 1173-1186; Zheng, P. P., Li, J., and Kros, J. M. 2018. “Breakthroughs in Modern Cancer Therapy and Elusive Cardiotoxicity: Critical Research-practice Gaps. Challenges, and Insights,” Medical Research Reviews 38(1): 325-376). That the irradiation of pelvic tissue has proven especially susceptible to collateral injury and ensuing initiation of mutagenesis resulting in the development of cancer is addressed above in this section.

The prevention of exposure in the first place is more effective than is the secondary administration of resveratrol and/or hesperidin, for example. Nevertheless, in conjunction with the immune tolerance induction incorporated into metered switch solid organ transplantation, the posttransplantation targeting of resveratrol and/or hesperidin, for example, directly to a transplant organ such as the heart should ward off the vasculopathy, which as addressed above in this section, is a frequent cause of late-term graft failure (Abdelgawad. I. Y., Grant, M. K. O., and Zordoky, B. N. 2019. “Leveraging the Cardio-protective and Anticancer Properties of Resveratrol in Cardio-oncology,” Nutrients 11(3). pii: E627; Dyck, G. J. B., Raj. P., Zieroth, S., Dyck, J, R. B., and Ezekowitz, J. A. 2019. “The Effects of Resveratrol in Patients with Cardiovascular Disease and Heart Failure: A Narrative Review,” Online, MDPI [Multidisciplinary Digital Publishing Institute] International Journal of Molecular Sciences (Basel, Switzerland) 20(4). pii: E904; Musa, A. E., Omyan, G., Esmaely, F., and Shabeeb, D. 2019. “Radioprotective Effect of Hesperidin: A Systematic Review,” Medicina (Kaunas, Lithuania) 55(7). pii: E370; Musa, A, E. and Shabeeb, D. 2019. “Radiation-Induced Heart Diseases: Protective Effects of Natural Products,” Medicina (Kaunas, Lithuania) 55(5). pii: E126; Fischer, N., Seo, E. J., and Efferth, T. 2018. “Prevention from Radiation Damage by Natural Products,” Phytomedicine 47:192-200; Zordoky, B. N., Robertson, I. M., and Dyck, J, R. 2015. “Preclinical and Clinical Evidence for the Role of Resveratrol in the Treatment of Cardiovascular Diseases,” Biochimica et Biophysica Acta 1852(6): 1155-1177; Carter, L. G., D'Orazio. J. A., and Pearson, K. J. 2014. “Resveratrol and Cancer: Focus on in Vivo Evidence,” Endocrine-related Cancer 21(3): R209-R225; Catalgol. B., Batirel. S., Taga. Y., and Ozer, N. K. 2012. “Resveratrol: French Paradox Revisited,” Online, Frontiers in Pharmacology 3:141; Pradeep, K., Ko. K. C., Choi, M. H., Kang, J. A., Chung, Y. J., Park, S, H. 2012. “Protective Effect of Hesperidin, a Citrus Flavanoglycone, against γ-Radiation-induced Tissue Damage in Sprague-Dawley Rats,” Journal of Medicinal Food 15(5): 419-427; Aggarwal, B. B., Bhardwaj, A., Aggarwal, R. S., Seeram, N. P., Shishodia, S., and Takada, Y. 2004. “Role of Resveratrol in Prevention and Therapy of Cancer: Preclinical and Clinical Studies,” Anticancer Research 24(5A): 2783-2840).

While the character of the relationship between heart disease and cancer, both promoted by obesity, smoking, a lack of exercise, poor sleep, metabolic dysfunction, hyperglycemia, hypertriglyceridemia, hypertension, and poor nutrition that includes much sugar and harmful fats to complicate the attribution of such factors to either or both of these disease processes as independent or related is subject to variability in a given patient, the association of chemotherapy and radiation used to eradicate cancer and cardiomyopathy is well established (see, for example, Chen, J., Ding. Y., Chen, M., Gau, J., Jen, N., and 9 others 2019. “Displacement Analysis of Myocardial Mechanical Deformation (DIAMOND) Reveals Segmental Susceptibility to Doxorubicin-induced Injury and Regeneration” Journal of Clinical Investigation Insight 4(8). pii: 125362; Gong, I. Y., Ong. G., Brezden-Masley, C., Dhir, V., Deva, D. P., and 9 others 2019. “Early Diastolic Strain Rate Measurements by Cardiac MRI in Breast Cancer Patients Treated with Trastuzumab: A Longitudinal Study,” International Journal of Cardiovascular Imaging 35(4): 653-662; Guha, A. I, Armanious, M., and Fradley, M, G. 2019. “Update on Cardio-oncology: Novel Cancer Therapeutics and Associated Cardiotoxicities,” Trends in Cardiovascular Medicine 29(1): 29-39; Guha, A., Dey, A. K., Armanious, M., Dodd, K., Bonsu, J., and 4 others 2019. “Health Care Utilization and Mortality Associated with Heart Failure-related Admissions among Cancer Patients,” European Society of Cardiology Heart Failure 6(4): 733-746; Jerusalem, G., Lancellotti, P., and Kim, S, B. 2019. “HER2+ [human epidermal growth factor receptor 2 positive] Breast Cancer Treatment and Cardiotoxicity: Monitoring and Management,” Breast Cancer Research and Treatment 177(2): 237-250; Kitani. T., Ong, S. G., Lam, C. K., Rhee, J. W., Zhang, J. Z., and 9 others 2019. “Human-induced Pluripotent Stem Cell Model of Trastuzumab-induced Cardiac Dysfunction in Patients with Breast Cancer,” Circulation 139(21): 2451-2465; Kostakou, P. M., Kouris, N. T., Kostopoulos, V. S., Damaskos, D. S., and Olympios, C, D. 2019. “Cardio-oncology: A New and Developing Sector of Research and Therapy in the Field of Cardiology,” Heart Failure Reviews 24(1): 91-100; Meijers, W, C. and de Boer, R, A. 2019. Op cit. above in this section; Ram, P., Tiu, A., Lo, K. B., Parikh, K., and Shah, M. 2019. “Trends in the Prevalence of Malignancy among Patients Admitted with Acute Heart Failure and Associated Outcomes: A Nationwide Population-based Study,” Heart Failure Reviews June 7; Ribeiro, M, L., Jorge, A. J, L., Nacif. M. S., and Martins, W, A. 2019. “Early Detection and Monitoring of Cancer Chemotherapy-related Left Ventricular Dysfunction by Imaging Methods,” Arquivos Brasileiros de Cardiologia 112(3): 309-316; Tadic. M., Genger, M., Cuspidi, C., Belyavskiy, E., Frydas, A., and 6 others 2019. “Phasic Left Atrial Function in Cancer Patients before Initiation of Anti-cancer Therapy,” Online, MDPI [Multidisciplinary Digital Publishing Institute] Journal of Clinical Medicine (Basel, Switzerland) 8(4). pii: E421; Totzeck, M., Mincu, R. I., Heusch, G., and Rassaf. T. 2019. “Heart Failure from Cancer Therapy: Can We Prevent It?,” European Society of Cardiology Heart Failure 6(4): 856-862; Unthank, J. L.2, Ortiz, M., Trivedi, H., Pelus. L. M., Sampson, C. H., and 7 others 2019. “Cardiac and Renal Delayed Effects of Acute Radiation Exposure: Organ Differences in Vasculopathy, Inflammation, Senescence, and Oxidative Balance,” Radiation Research 191(5): 383-397; Bergamini. C., Dolci, G., Rossi, A., Torelli, F., Ghiselli, L., and 8 others 2018. “Left Atrial Volume in Patients with HER2-positive Breast Cancer: One Step Further to Predict Trastuzumab-related Cardiotoxicity,” Clinical Cardiology 41(3): 349-353; Choe, J. C., Choi, J. H., Choi, J. H., Ahn, J., Park, J. S., and 5 others 2018. “Prolonged Electromechanical Delay as an Early Predictor of Trastuzumab-induced Cardiotoxicity in Patients Undergoing Treatment for Breast Cancer,” Clinical Cardiology 41(10): 1308-1314; Clasen, S, C. and Wald, J. W. 2018. “Left Ventricular Dysfunction and Chemotherapeutic Agents,” Current Cardiology Reports 20(4): 20; Coviello, J. S. 2018. “Cardiovascular and Cancer Risk: The Role of Cardio-oncology,” Journal of the Advanced Practitioner in Oncology 9(2): 160-176; Delgobo, M. and Frantz, S. 2018. “Heart Failure in Cancer: Role of Checkpoint Inhibitors,” Journal of Thoracic Disease 10 (Supplement 35): S4323- S4334; Henriksen, P, A. 2018. “Anthracycline Cardiotoxicity: An Update on Mechanisms, Monitoring, and Prevention,” Heart (British Cardiac Society) 104(12): 971-977; Quatromoni, N, and Scherrer-Crosbie, M. 2018. “Update on Incorporating Biomarkers with Imaging Findings for the Detection and Management of Cardiotoxicity,” Current Cardiology Reports 20(8): 67; Rassaf. T. and Totzeck, M. 2018. “Modern Concepts in Cardio-oncology,” Journal of Thoracic Disease 10 (Supplement 35): S4386-S4390; Venneri, L., Zoppellaro, G., and Khattar, R. S. 2018. “Cardio-oncology: The Role of Advanced Echocardiography in Cancer Patients,” Expert Review of Cardiovascular Therapy 16(4): 249-258; Henning, R. J. and Harbison, R, D. 2017. “Cardio-oncology: Cardiovascular Complications of Cancer Therapy,” Future Cardiology 13(4): 379-396; Hernandez-Suarez, D, F. and Lopez-Candales, A. 2017. “Potential Usefulness of Diastolic Parameters Measured by Strain Imaging Echocardiography in the Early Prediction of Chemotherapy-induced Cardiotoxicity,” Medical Hypotheses 101:30-32; Klee, N. S., Mccarthy, C. G., Martinez-Quinones, P., and Webb, R. C. 2017. “Out of the Frying Pan and Into the Fire: Damage-associated Molecular Patterns and Cardiovascular Toxicity following Cancer Therapy,” Therapeutic Advances in Cardiovascular Disease 11(11): 297-317; Paraskevaidis, I. A., Makavos, G., Tsirigotis, P., Psarogiannakopoulos, P., Parissis, J., and 3 others 2017. “Deformation Analysis of Myocardial Layers Detects Early Cardiac Dysfunction after Chemotherapy in Bone Marrow Transplantation Patients: A Continuous and Additive Cardiotoxicity Process,” Journal of the American Society of Echocardiography 30(11): 1091-1102; Tajiri, K., Aonuma, K., and Sekine, I. 2017. “Cardio-oncology: A Multidisciplinary Approach for Detection, Prevention, and Management of Cardiac Dysfunction in Cancer Patients,” Japanese Journal of Clinical Oncology 47(8): 678-682; Yusuf, S. W., Venkatesulu, B. P., Mahadevan, L. S., and Krishnan, S. 2017. “Radiation-induced Cardiovascular Disease: A Clinical Perspective,” Online, Frontiers in Cardiovascular Medicine 4:66; Curigliano, G., Cardinale, D., Dent, S., Criscitiello, C., Aseyev, O., Lenihan, D., and Cipolla, C. M. 2016. “Cardiotoxicity of Anticancer Treatments: Epidemiology, Detection, and Management,” CA [American Cancer Society Journal for Clinicians] 66(4): 309-325; Lenneman, C, G. and Sawyer, D, B. 2016. “Cardio-oncology: An Update on Cardiotoxicity of Cancer-related Treatment,” Circulation Research 118(6): 1008-1020; Tapio, S. 2016. “Pathology and Biology of Radiation-induced Cardiac Disease,” Journal of Radiation Research 57(5): 439-448; Virani, S. A., Dent, S., Brezden-Masley, C., Clarke, B., Davis, M. K., and 20 others 2016. “Canadian Cardiovascular Society Guidelines for Evaluation and Management of Cardiovascular Complications of Cancer Therapy,” Canadian Journal of Cardiology 32(7): 831-841).

Turning now to vascular valves, between bistable solenoid valves for one-time use in sudden compound vascular bypass, or sudden switched-organ transplantation and urinary prostheses, and continuously adjustable servovalves for use in metered compound vascular bypass, or sudden switched-organ transplantation, both addressed below, stand manually controlled valves. Like bistable valves, these are adjusted to be fully open or closed, but can also be adjusted to any position between the two. These are used to divert urine directly from the ureters and into an external collection bag during the night, then be switched to reinstate normal drainage into the bladder, thus preventing the interruption of sleep by the need to urinate.

To replace a resected or malformed lower urinary tract, the need to urinate is automatically signaled and/or controlled as a prosthesis. Devised to avoid injury to the substrate ductus, vascular valves and servovalves are lined with viscoelastic foam to protect vasa vasorum and nervorum, while in use, only the diversion chute is inserted into the substrate lumen. The structure and use of basic ductus side-entry jackets are described and illustrated in U.S. Pat. No. 11,759,186, and nonjacketing side-entry connectors in U.S. Pat. No. 11,013,858.

As indicated, a luminal flow diversion side-entry jacket is a side-entry jacket as described in U.S. Pat. No. 11,759,186, titled Ductus Side-entry Jackets and Prosthetic Disorder Response Systems adapted to be adjustable. As such, it can incorporate any of the features of side-entry jackets generally. These include the incorporation between the inside of the jacket shell and foam lining of tiny bioprotectively encapsulated neodymium iron boron alloy permanent magnets, or to allow on-off and graduated intensity, implanted timing module or microcontroller-controlled electromagnets, which have been magnetized or oriented to project an attractive field perpendicular to the axis of the native lumen to which the jacket is fastened in encircling relation.

The jacket can therefore function at the same time as an impasse jacket using neodymium permanent magnets magnetized, or electromagnets wound to project an attractive field perpendicular to the axis of the substrate ductus as also described in U.S. Pat. No. 11,759,186. More specifically, a diversion jacket incorporating permanent or electromagnets can be used as an impasse-jacket to stop a superparamagnetic iron oxide nanoparticle-carried drug or drugs against the luminal wall. Because electromagnets allow on-off and adjustable field strength, these can be used with greater flexibility as detention jackets to draw such drugs against the lumen lining or by increasing the field strength, as incorporation jackets to draw such drugs into the lumen wall.

The use of a conventional side-entry or diversion jacket determined by the concurrent need to divert some portion of the flow, when access to the target site is blocked by bone, as with Ewing's sarcoma, the major blood supply artery is jacketed or the bone drilled with the side-entry magnetized on the contralateral side to give direct access to the red marrow of allogeneic hemopoietic stem cells. Other jackets can be strategically positioned proximal to the site for direct targeting of immunosuppressant drugs, the systemic dose then considerably reduced if not eliminated.

A side-entry jacket on the blood supply allows the direct targeting of immunosuppressant drugs to a solid organ transplant. A side-entry diversion jacket allows flow through the native ductus the jacket encircles to be reduced no more than is necessary to draw off a diagnostic sample through a line to the surface port. If outflow is lacking, a fine catheter can be passed through the body surface port, mainline or accessory channel, and into the native lumen to obtain the sample. Magnetized ductus side-entry jackets and diversion jackets positioned along the aorta, ureters, and larger arteries to deliver susceptible drug-carried rituximab and/or steroids locally in higher concentration than if dispersed systemically to treat local lesions should materially reduce the effects of IgG4-related disease without the serious side effects such as moon facies and infusion reactions.

Such reactions include pain, infection, and embolism (see, for example, Lei, W. H., Xin, J., Shao, C. X., Mao, M. F., Zhu, C. Y., Wu, C. F., and Jin, L. 2016. “IgG4-related Kidney Disease Mimicking Malignant Ureter Tumor: Case Report and Literature Review,” Medicine (Baltimore). 95(3): e2550; Akiyama, M., Kaneko, Y., Hayashi, Y., and Takeuchi, T. 2016. “IgG4-related Disease Involving Vital Organs Diagnosed with Lip Biopsy: A Case Report and Literature Review,” Medicine (Baltimore) 95(24): e3970; Mahajan, V. S., Mattoo, H., Deshpande, V., Pillai, S. S., and Stone, J, H. 2014. “IgG4-related Disease,” Annual Review of Pathology 9:315-347; Marando, A., D'Ambrosio, G., Catanzaro, F., La Rosa, S., and Sessa, F. 2013. “IgG4-related Disease of the Ureter: Report of Two Cases and Review of the Literature,” Virchows Archiv 462(6): 673-678).

A side-entry jacket incorporating small electromagnets positioned just proximal or distal to a bifurcation in the arterial tree where high shear stress promotes the development of atherosclerotic lesions can thus be used to draw remedial drugs in magnetically susceptible form flowing past it into the lesions, for example. A side-entry diversion jacket, which is invulnerable to shear stress, similarly equipped can be positioned just proximal to the bifurcation to allow resection of a segment of an unsalvageable branch of arm of the bifurcation, and deliver medication into the remaining salvageable segment. The value of synthetic prostheses in the vascular tree is greatest in patients with systemic vascular disease who are unable to provide a suitable autologous graft and those debilitated in whom graft harvesting autologous or homologous would be ill advised.

A conventional drug, drugs, or ferrofluid containing a drug or drugs may be injected through a conventional syringe, jet injector (pneumatic injector, jet gun injector), insulin pump, central venous catheter into the body surface port opening or openings of the accessory channel or channels leading to a conventional or a flow diversion side-entry jacket, where the jacket is positioned superior (cephalad, craniad, upward) to the diversion jacket, or through an accessory channel or channels of the diversion jacket itself. The jet injector shown in FIG. 27B is discharged by releasing a spring rather than by compressed gas, and used only to replenish the drug reservoirs, requires no protective cap to prevent contamination. Ductus side-entry jackets and side-entry diversion jackets can also position sensing probes with self-contained diagnostic electronics or electronics implanted in a different location to which the probe is connected in a fibrous encapsulation-resistant location which allows diagnostic data to be obtained in vivo without the awareness of the patient.

To allow medication or facilitated viewing of the junction created, both reducing if not eliminated exposure to unintended tissue, the jacket is provided with at least one accessory channel, which is a line subsidiary to the mainline, a sideline, with side entry to the mainline connected along the jacket side connector. An accessory channel allows the directly pipeline targeted delivery into the jacket and the substrate ductus to which it is connected of any fluid agent. The utility of an accessory channel is critical, allowing the directly targeted delivery of drugs, medicinal solutions, or contrast into side-entry jackets, side-entry jacket-based extravascular and intravascular choke valves and choke servovalves, diversion valves and servovalves, as well as nonjacketing side-entry connectors and the substrate vessel to which the device is connected.

This not only facilitates observation of the treatment site, but the direct delivery of drugs to suppress any adverse reaction to jacketing the ductus might present and/or allow the directly targeted release of drugs to treat any downstream disease at any later date. A small port at the body surface, much like a larger single port mediport or portacath, and similarly positioned subdermally in the pectoral region, incorporates the number of entry points for different drugs, or for a frail patient, a number of additional entry ports for future use when and if needed. Tiny tattooed marks on the skin indicate the transcutaneous injection or infusion points. A urinary diversion line to a paracorporeal collection bag is positioned in an inferior location, preferably to a side of the mons pubis, with only the port opening at the body surface cutaneous, or above-skin.

The structure and function of side-entry flow diversion jackets is described and illustrated in U.S. Pat. No. 11,759,186, entitled Ductus Side-entry Jackets and Prosthetic Disorder Response Systems, and side-entry vascular servovalves in the copending parent application hereto of like title, Ser. No. 16/873,914. In such valves, a circular water jacket, just inside and concentric with the core-cutting trepan through which a vacuum if applied to the adventitia, is first used to discharge a circular water jet to prevent the extravasation of blood through the entry hole when cutting into a blood vessel. The water jet also repels and thus protects the endothelium or urothelium opposite the entry hole from the trepan cutting edge.

Then, once the jacket has been positioned, the same inner water jacket serves as a pathway, the sideline or accessory channel, to deliver drugs in fluid form and medicinal solutions in the antegrade direction into the lumen of the substrate ductus to which the jacket is connected. The lumen of the trepan tube, or mainline, which conveys blood or urine, can be used in the antegrade direction to deliver fluids into the ductus lumen or in the retrograde direction as an outlet to pass urine, for example.

Citations to the literature provided both above and below, synthetic-organic interfaces can be prepared to passively resist adverse tissue reactions through the use of 1. Materials embedded with reaction-counteractive nanoparticulates, and/or 2. An outer nanometric coating of a highly elastic material such as of thin vapor-deposited copolymerized (see, for example, Galeotti, F., Andicsova, A., Bertini, F., Laux, E., Hartmann, L., and 6 others 2014. “Enhanced Elasticity in Parylene Thin Films by Copolimerization Approach,” Journal of Materials Science 49(21): 7547-7555; Lendlein, A. and Langer, R. 2002. “Biodegradable, Elastic Shape-memory Polymers for Potential Biomedical Applications,” Science 296(5573): 1673-1676) and plasticized parylene embedded with an antimicrobial (references below), 3. The same additionally impregnated with a similar coating film including embedded nanoparticulate agents to combat infection and inflammation, and 4. At the surface of a solid organ where an adverse tissue reaction were it to occur would be of short duration, microneedle patches, especially those absorbable, can substitute for more costly methods for targeting an anti-inflammatory and/or antimicrobial to the interface on a replenishable basis.

Active resistance is by the direct targeting to the site requiring treatment of remedial agents such as anti-inflammatoires and antimicrobials, through accessory channels, or if required at minute locations such as the adventitial-foam lining of side-entry jackets and connectors, through a capillary gauge soft polymer walled tube with numerous perforations in the sides to ‘bleed’ and wet the interface. In arteries, where a thin micromillimetric film lining the foam must incorporate micropores, the line or lines can pass through the foam to ‘bleed’ out of the pores into the interface.

Biomaterials with integral nanoparticulates to counteract adverse tissue, or foreign body reactions are addressed elsewhere in this section. Special cases where, for example, an immunosuppressant is targeted to the jacket to increase susceptibility to infection, may justify the directing of an accessory channel, of which more than one can be connected to the jacket side-tube, through capillary gauged tubing into the adventitia-foam interface to release a steroid or antimicrobial, for example.

Albeit very small, except for use with arteries where complete enclosure induces atherosclerotic degeneration, perforation of the Parylene® (Diamond-MT [owner initials], Johnstown, Pennsylvania) or a similar coating to allow the antimicrobial or anti-inflammatory to ‘bleed’ into the interface is not preferred. Any adverse tissue reaction or degenerative alteration at the jacket lining foam-adventitial interface is directly targetable into the interface with inflammation and antimicrobial counteractants. Damped nonsparking solenoid-driven valves are bistable in completely passing flow without diversion or completely diverting flow, whereas manually and linear motor-driven servovalves controlled by an implanted microcontroller can maintain as undiverted or divert any fraction of flow.

For temporary use, microneedle patches offer the simplest and least costly means for suppressing an initial short term adverse tissue reaction at the tissue surface-implant interface (see, for example, He. X., Sun, J., Zhuang, J., Xu. H., Liu, Y., and Wu, D. 2019. “Microneedle System for Transdermal Drug and Vaccine Delivery: Devices, Safety, and Prospects,” Dose-Response 17(4): 1559325819878585; Zhang, L. and Wei. W. 2019. “Anti-inflammatory and Immunoregulatory Effects of Paeoniflorin and Total Glucosides of Paeony,” Pharmacology and Therapeutics 10:107452; Yang, J., Chen, Z., Ye. R., Li, J., Lin, Y., and 4 others 2018. “Touch-actuated Microneedle Array Patch for Closed-loop Transdermal Drug Delivery,” Drug Delivery 25(1): 1728-1739).

Where the substrate structure is an organ with a substantial protective outer fibrous layer or a ductus other than an artery, microneedle patches can be used to deliver medication to the jacket-adventitia interface. The medication of a conventional microneedle patch nonreplenishable, these are suited to the suppression of adverse reactions brief in duration. The patch is best perforated, and to not leave behind material that could only be the cause of complications, absorbable.

Accordingly, where an adverse reaction is time limited, absorbable polymeric microneedles suffice. The limitation in duration of drug delivery may be overcome by stacking the dissolvable microneedle patches in layers on absorbable backings in a compound patch (Lau. S., Fei, J., Liu, H., Chen, W., and Liu, R. 2016. “Multilayered Pyramidal Dissolving Microneedle Patches with Flexible Pedestals for Improving Effective Drug Delivery,” Journal of Controlled Release pii: S0168-3659(16) 30601-0; Hong, X., Wei, L., Wu. F., Wu Z., Chen, L., Liu, Z., and Yuan, W. 2013. “Dissolving and Biodegradable Microneedle Technologies for Transdermal Sustained Delivery of Drug and Vaccine,” Drug Design, Development, and Therapy 7:945-952).

Nanometric in proportions, polymer microneedles facilitate the transport of medication across the skin, and could do so across the fibrosa of an organ when applied to patches which load medication suitable for topical application in internal use (see, for example, Sivaraman, A. and Banga, A. K. 2017. “Novel in Situ Forming Hydrogel Microneedles for Transdermal Drug Delivery,” Drug Delivery and Translational Research 7(1): 16-26; Ma, G. and Wu, C. 2017. “Microneedle, Bio-microneedle and Bio-inspired Microneedle: A Review,” Journal of Controlled Release 51:11-23; Ita, K. 2015. “Transdermal Delivery of Drugs with Microneedles-Potential and Challenges,” Pharmaceutics 7(3): 90-105; Donnelly, R. F., McCrudden, M. T., Zaid Alkilani, A., Larrañeta, E., McAlister, E., and 8 others 2014. “Hydrogel-forming Microneedles Prepared from “Super Swelling” Polymers Combined with Lyophilized Wafers for Transdermal Drug Delivery,” Online, PLoS [Public Library of Science] One 9(10): e111547; Donnelly, R. F., Mooney, K., McCrudden, M. T., Vicente-Perez, E. M., and 4 others 2014. “Hydrogel-forming Microneedles Increase in Volume during Swelling in Skin, but Skin Barrier Function Recovery is Unaffected,” Journal of Pharmaceutical Sciences 103(5): 1478-1486; Demir, Y. K., Akan, Z., and Kerimoglu, O. 2013. “Characterization of Polymeric Microneedle Arrays for Transdermal Drug Delivery,” Online, PLoS [Public Library of Science] One 8(10): e77289; Kim, M., Jung. B., and Park, J, H. 2012. “Hydrogel Swelling as a Trigger to Release Biodegradable Polymer Microneedles in Skin,” Biomaterials 33(2): 668-678; Prausnitz, M, R. 2004. “Microneedles for Transdermal Drug Delivery,” Advanced Drug Delivery Reviews 56(5): 581-587; McAllister, D. V., Wang, P. M., Davis, S. P., Park, J. H., Canatella, P. J., Allen, M. G., and Prausnitz, M, R. 2003. “Microfabricated Needles for Transdermal Delivery of Macromolecules and Nanoparticles: Fabrication Methods and Transport Studies,” Online, Proceedings of the National Academy of Sciences of the United States of America 100(24): 13755-13760).

As shown in FIGS. 3 and 6, ductus side-entry flow diversion valve or servovalve incorporates a diversion chute 18 mounted on a slideway or track 21 which allows the chute with integral ostium or valve outlet obturator 30 at its front end to be advanced from within the trepan tube 5 within the side stem 19 forward into shape-compliant contact with the opposite side of the lumen wall 14 to switch flow away from the lumen and divert it through the trepan tube 5 within the side stem 19 into the shunt or bypass connected to the side stem 19. The details of ductus side-entry diversion jacket and servovalve construction are provided in section 6, entitled Description of the Preferred Embodiments of the Invention.

As shown in FIGS. 3 and 6, ductus side-entry flow diversion valve or servovalve incorporates a diversion chute 18 mounted on a slideway track 21 which allows the chute with integral ostium or valve outlet obturator 30 at its front end to be advanced from within the trepan tube 5 within the side stem 19 forward into shape-compliant contact with the opposite side of the lumen wall 14 to switch flow away from the lumen and divert it through the trepan tube 5 within the side stem 19 into the shunt or bypass connected to the side stem 19. The details of ductus side-entry diversion jacket and servovalve construction are also provided in section 6, entitled Description of the Preferred Embodiments of the Invention.

To self-deploy, or self-unfold, ostium obturator 30 is made of a shape-memory polymer (see, for example, Pongwisuthiruchte, A., Aumnate, C., and Potiyaraj. P. 2024. “Tailoring of Silicone Urethane Methacrylate Resin for Vat Photopolymerization-based 3D Printing of Shape Memory Polymers,” Online, American Chemical Society Omega (Washington, D.C.) 9(2): 2884-2895; Staszczak, M., Urbański, L., Cristea, M., Ionita, D., and Pieczyska, E, A. 2024. “Investigation of Shape Memory Polyurethane Properties in Cold Programming Process Towards Its Applications,” Online, MDPI [Multidisciplinary Digital Publishing Institute] Polymers (Basel, Switzerland) 16(2): 219; Ramezani. M., Labour, E. E., Ji, J., Vakil, A. U., Du, C., and 3 others 2023, Op cit. above in section 2a, entitled Background of the Invention. General, in the subsection thereof entitled Materials and Surface Treatment to Repel Microbial Colonization; Sarrafan, S. and Li, G. 2023. “On Lightweight Shape Memory Vitrimer Composites,” Online, American Chemical Society Applied Polymer Materials (Washington, D.C.) 6(1): 154-169; Zhao, Q., Zou, W., Luo, Y., and Xie. T. 2016. “Shape Memory Polymer Network with Thermally Distinct Elasticity and Plasticity,” Science Advances 2(1): e1501297; Hoffman, A. S. 2013. “Stimuli-responsive Polymers: Biomedical Applications and Challenges for Clinical Translation,” Advanced Drug Delivery Reviews 65(1): 10-16; Serrano, M, C. and Ameer, G, A. 2012. “Recent Insights into the Biomedical Applications of Shape-memory Polymers,” Macromolecular Bioscience 12(9): 1156-1171; Lendlein, A., Behl, M., Hiebl. B., and Wischke, C. 2010. “Shape-memory Polymers as a Technology Platform for Biomedical Applications,” Expert Review of Medical Devices 7(3): 357-379; Sokolowski, W., Metcalf, A., Hayashi, S., Yahia, L., and Raymond, J. 2007. “Medical Applications of Shape Memory Polymers,” Biomedical Materials (Bristol, England) 2(1): S23-527; Yakacki, C. M., Shandas, R., Lanning, C., Rech, B., Eckstein, A., and Gall, K. 2007. “Unconstrained Recovery Characterization of Shape-memory Polymer Networks for Cardiovascular Applications,” Biomaterials 28(14): 2255-2263; Mohr, R., Kratz, K., Weigel. T., Lucka-Gabor, M., Moneke, M., and Lendlein, A. 2006. “Initiation of Shape-memory Effect by Inductive Heating of Magnetic Nanoparticles in Thermoplastic Polymers,” Online, Proceedings of the National Academy of Sciences of the United States of America 103(10): 3540-3545; Lendlein, A. and Kelch, S. 2002. “Shape-Memory Polymers,” Angewandte Chemie International Edition in English 41(12): 2034-2057).

Voluntary control of adjustable ureteral takeoff urinary diversion jackets is by the wearer who rotates a knob on a small surface port in the lower pelvic region, to move the Bowden, or push-pull cables that slide the diversion chutes. The surface port is usually fastened to a side of the mons pubis and also contains the outflow opening for connection to the collection bag of a drainage tube. Ureteral takeoff eliminates filling of the bladder, hence, urge sensation. Where this poses no problem, takeoff can be from the bladder, allowing reduction in the valves and cables to one.

In many cases, a unilateral ureteral takeoff with the contralateral ureter draining into the bladder will afford protection over the interval required. Positioned to one side of the mons pubis, the surface port such as that shown in FIG. 26, incorporates the opening through which urine passes through a hose into the collection bag. When facilities are nearby, the valves can be switched from diverted to normal, or urethral, outflow. Prior to a public performance, for example, the incontinent user switches the valve or valves to divert urine from the ureters to the collection bag. The implantation of a voluntary urine evacuation system is properly responsive only to chronic, not short-term conditions such as frequent urination due to a lower urinary tract infection readily dispelled by taking an oral antibiotic.

Any urinary voidance prosthesis such as that shown in FIG. 28 or assist system such as that shown in FIG. 30 can be unilateral or bilateral. With a combination outflow/inflow port such as those shown in FIGS. 26C and 28, drugs can be injected into the surrounding drug entry openings; however, inferior, or caudal to the renal pelvises, or pelves, a small pump such as peristaltic positioned close to the floor of the peritoneal cavity, for example, is necessary to move the drug against the force of gravity through the druglines leading into the drainage diversion jacket or valve accessory channels.

If preferred as presaging discomfort or consuming additional power, this can be avoided by introducing drugs at the pectoral level through a second supportive port of the kind shown in FIGS. 26A and 26B, so that delivery is by gravity. Chronic disease of one or both kidneys is treated by positioning basic side-entry jackets such as described and illustrated in U.S. Pat. No. 11,759,186, entitled Ductus Side-entry Jackets and Prosthetic Disorder Response Systems, along the renal artery or arteries and delivering the medication through the accessory channels thereof.

Neoplasms within the renal parenchyma are treated with the aid of nonjacketing side-entry connectors as described and illustrated in U.S. Pat. No. 11,013,858, entitled Nonjacketing Side-entry Connectors and Prosthetic Disorder Response Systems. Such connectors maintain a therapeutic probe, for example, correctly aimed as the lesion despite movement of the organ treated. Disease affecting the walls of the renal arteries or veins are treated by positioning radially magnetized perivascular side-entry jackets about these to draw superparamagnetic particle-carried medication radially outward from the passing blood into the vascular wall as described and illustrated in U.S. Pat. No. 11,389,171 entitled Integrated System for the Infixion and Retrieval of Implants.

Adaptive control over vascular (nonurinary) servovalves is electrical, in a fully implanted system under the control of a microcontroller, or in comorbid disease, a microprocessor, and can be overridden only by a clinician having access to the microcontroller or microprocessor password through a secure Online database or patient medical record. In sudden compound vascular bypass, or sudden switched-organ transplantation, the diversion of blood requires bistable control-full flow with the chute fully extended and none with the chute fully retracted. In metered compound vascular bypass, or sudden switched-organ transplantation, control of the diversion valves is continuously variable under the control of the implanted master control microprocessor in response to data input streams fed to it from implanted physiological sensors.

Where the diversion jackets are to remain and controllably apportion flow between a bypass or shunt and the native vessel, control is generally responsive to feedback from implanted physiological sensors placed at separate locations in the body. Servocontrol by the prescription-programmed microcontroller is typically by continuous adjustment of small linear motor driven diversion chute valve-jackets, or vascular servovalves. Automatic monitoring and response with valve adjustment and the directly targeted release of drugs is essential for ischemia-free metered compound vascular bypass, or sudden switched-organ transplantation of solid organs with concurrent immune tolerance induction, the subsequent maintenance thereof, and is especially important in double heart transplantation.

The side-stem or trepan end-piece through which flow is diverted through the jacket shell and into the mainline is the side connector, and distinct from nonjacketing side-entry connectors as described in U.S. Pat. No. 11,013,858. If blood, the mainline conducts the effluent through the side connector and into the lumen of the destination vessel-mainline to mainline, through the side connector of the receiving jacket and into the native lumen. If urine, the mainline conducts the effluent to and through the side connector and into the lumen of the ipsilateral or contralateral ureter, or to and through a nonjacketing side-entry connector on the bladder, or to a paracorporeal collection bag.

The term ‘downstream’ means past the structure of reference in the antegrade direction. In the head (caput), downstream arterial blood flow is craniad (cephalad, upward) and upstream venous flow caudad (downward). A side-entry diversion jacket is unrelated to a conventional vascular or urinary diversion stent, or “diversistent,” which is used to preserve the integrity of the native ductus such as by preventing the diversion of flow through a nonsurgical fistula.

Where a side-entry jacket is essentially an implantable bisphenol A and residual plasticizer-free polymeric conduit fitting, molded or fabricated in the form of a tee or a wye-joint with unitized stem fixed to any angle relative to the jacket barrel, an adjustable or nonprosthetic side-entry diversion jacket additionally incorporates a continuously deployable curved chute, or tongue, which according to the extent of chute deployment into the native lumen, diverts flow away from the lumen of the jacketed or encircled substrate native ductus.

A choke valve jacket or side-entry diversion valve jacket is not positioned along the length of a catheter such as a mainline (bloodline, urine line) or a sideline (accessory channel, drugline, drug feedline) to adjust or continuously modulate the delivery of a drug through the line. Rather, the jackets connect the lines at the origin and terminus. Drug delivery and valve adjustment are controlled by an implant microcontroller in less complex disease or microprocessor in comorbid disease. This closed loop negative feedback control responds to data supplied to the controller from implanted physiological parameter sensors.

When the indicia indicate the need, in accordance with its preferably personalized prescription-program, the control device adjusts the diversion valves and targets medication to the site. An internal drip drugline to wet the foam lining in order to alleviate any inflammation, typically with the aid of a steroid such as prednisolone, triamcinolone, cortisone, or an ameliorant such as aloe vera, released into the irritated interface is advised for incorporation into any jacket likely to arouse such an adverse tissue response.

Drug delivery is by control of the outlet pump or pumps or drip stopcocks of small flat reservoirs subcutaneously, or subdermally, positioned in the pectoral area, usually injected into and gravity-fed from a small body surface port for controlled released by the system microcontroller or microprocessor through one or more jacket accessory channels. Basic ductus side-entry jackets and side-entry diversion valves or servovalve-jackets are positioned in surrounding or collaring relation to the substrate native ductus, not at junctions, such as at the incurrent and excurrent ends of surgical bypass anastomoses.

Entry into the native lumen is gained through an opening or ostium cut in the side of the native ductus by the razor-sharp leading-edge trepan at the adductal (near to the ductus, not related to adductor) end of the extendable and retractable side-stem side connector mainline tube which cuts an opening or ostium in the side of the native ductus, so that the lumen of the side connector, or side-stem, is made continuous with that of the native ductus. A benefit of the jacket is a reduction in the intimacy of interconnection of direct synthetic material-to-native tissue contact such as the suture line along an anastomosis, where the synthetic is repeatedly run entirely through the wall of the ductus. To ameliorate an adverse tissue reaction over the foam-adventitia contact interface, the foam lining is impregnated with a nonsteroid steroid anti-inflammatory such as curcumin or a steroid anti-inflammatory such as dexamethasone, prednisolone, prednisone, cortisone, of triamcinolone, and to ward off infection, an antimicrobial.

The opening, or surgical ostium, is created by the extraction from the side of the ductus of a plug of tissue the same in diameter as the native lumen. To do this, a vacuum pump is connected to the side-stem side connector mainline tube. The application of suction to the side wall of the ductus through the mainline side tube with trepan against the wall draws the tissue inside the trepan into the side tube. Gouging injury to the internal wall of the ductus opposite the opening is prevented by the vacuum pump limit switch, which stops in response to the sudden loss of resistance to the vacuum at the instant the opening is completed. The vacuum can then be used to assist in extracting the plug.

Should the plug catch on the front end of the chute so that the momentary vacuum surge does not draw it out, the plug is removed with the aid of a small caliber, or capillary gauge, suction tube. U.S. Pat. No. 11,759,186, entitled Ductus Side-entry Jackets and Prosthetic Disorder Response Systems, also shows a hook-ended guidewire that can inserted into the side tube to extract the plug. To aid visibility, the side-stem side connector mainline tube trepan leading edge is marked with contrast, and the polymeric parts, typically made of endocrine disrupter and residual plasticizer-free polyetheretherketone (PEEK, Invibio Biomaterial Solutions Division, Victrex, Public Limited Company, Thomton Cleveleys, Lancashire, England).

Alternative polymers are numerous and include optically transparent silicone and poly(acrylonitrile-butadiene-styrene) nanocomposites modified with silver nanoparticles (Ziąbka, M., Dziadek, M., and Pielichowska, K. 2020. “Surface and Structural Properties of Medical Acrylonitrile Butadiene Styrene Modified with Silver Nanoparticles,” Polymers (Basel, Switzerland) 12(1). pi: E197; Ziąbka, M. and Dziadek, M. 2019. “Long-term Stability of Two Thermoplastic Polymers Modified with Silver Nanoparticles,” Nanomaterials (Basel, Switzerland) 9(1). pii: E61; Ziąbka, M. and Dziadek, M. 2019. “Long-lasting Examinations of Surface and Structural Properties of Medical Polypropylene Modified with Silver Nanoparticles,” Polymers (Basel, Switzerland) 11(12). pii: E2018; Ziąbka, M., Dziadek, M., and Menaszek, E. 2018. “Biocompatibility of Poly(acrylonitrile-butadiene-styrene) Nanocomposites Modified with Silver Nanoparticles,” Polymers (Basel, Switzerland) 10(11). pii: E1257).

A side-entry diversion jacket can be used to divert flow from the artery it encircles to grasp about through its side connector and the catheter, or mainline, connected thereto, to bypass an aneurysmal, traumatized, diseased, debilitated, atherosclerotic, or healing downstream segment of the artery before returning flow to the artery through an upstream return or reentry side-entry diversion jacket placed to encircle the artery beyond or downstream to the defect. As to terminology, the bypass line is connected to the side connector in both the diversion and reentry jackets, and a sideline enters the side connector to deliver drugs through the side connector and into the lumen.

Using a conventional side-entry jacket with angled side connector as described and illustrated in U.S. Pat. No. 11,759,186, FIGS. 19 thru 22, sized to tap off blood at the proper flow rate, or an adjustable side-entry diversion jacket with the chute extended to the proper distance to like effect, it is also possible to initiate the bypass at a point along any larger artery such as the thoracic or abdominal aorta; the bypass can thus be of any length and follow any course that will not strangulate an intervening structure.

If collateral surgery is needed, this allows the origin of the bypass to be positioned where it is least likely to interfere. Increase in length of the bypass does, however, increase the rate of anticoagulant drip required. If the bypass must remain, the synthetic bypass is used as a prosthesis with a small port at the body surface through which to inject an anticoagulant into the jacket of the bypass initiating jacket. Except for the relatively brief interval during which the bypass is applied, there is little if any change in blood pressure through the internal and external carotids.

If the port at the body surface is to house a battery, it must be cutaneous; if transdermal energy transfer is used, the port is subdermal. The battery powers a small pump at the outlet of a flat reservoir, usually in the pectoral region, refilled by injection through the port, the pump controlled by an implanted microcontroller, the prosthesis then fully implanted. In general, whereas a nonadjustable side-entry diversion jacket is fully extended to close off the bloodstream, partially extending the chute of an adjustable side-entry diversion jacket into the native lumen allows the diversion of blood that is partial. As well as allowing the origin of a bypass along any large artery, this capability can be used to adjust the blood pressure through the bypass and the segment of the artery affected.

However, barring a medical contraindication, it is less complicated to reduce the pressure by directly targeting a suitable drug into the native lumen through an accessory channel of the diversion jacket. To avert shear stress, bypass around the defective segment is to a downstream side-entry jacket with side connector angled toward the direction of blood return. If the bypass takeoff diversion jacket is adjustable, the chute can be slightly withdrawn to lower the blood pressure through the bypass, or its accessory channel used to deliver a topical vasoconstrictor to increase the pressure. Use of the same or a different accessory channel is used to drip an anticoagulant into the line from a subdermally placed flat reservoir through control of its pump by a microcontroller likewise fully implanted.

Targeting the anticoagulant to the treatment site in a dose too small to affect the systemic circulation protects the patient from problem bleeding in the event of an accident and/or the need for emergency surgery. Drugs delivered in like manner where uptake within the target segment is incomplete and might pose some risk if circulated are neutralized or counteracted through the release of a reversal agent if available by the upstream side-entry jacket or jackets. For this reason, the anticoagulant of choice at this time is warfarin. In an open procedure, control of the chute to include adjustment if appropriate is manual by the operator, anesthetist, or perfusionist.

Whether the bypass jackets and line are removed depends upon the circumstances. If not to be left in place indefinitely, these are removed before closing. Adjustability by a competent patient once closed is by turning a control knob on a small cutaneously placed body surface port the size of a small button connected to the chute by means of a miniature push-pull cable control cable. If the patient is not competent, then control of the chute if appropriate is by radio remote control, eliminating the need for a surface port with control knob and push-pull control cable, requiring instead a highly miniaturized, fully implanted, transdermal receiver and antenna, the chute then moved by a tiny a fully implanted microcontroller following a prescription-program through a tiny linear servomotor.

Thus, by slightly retracting the chute or infusing an anticoagulant, the blood bypassed can be lowered, restored to, or elevated in full mean arterial pressure. A remotely controlled adjustable side-entry diversion jacket positioned along an artery can be used as an electrovalve to control the volume of flow diverted through the catheter, thus differentially apportioning with continuous variability, the blood, hence, the blood pressure, flowing through the native and catheteric lines. This pressure can be dynamically adjusted by an implanted microcontroller responsive to sensors used to detect physiological parameters implanted in the jackets and/or elsewhere in the body.

An aneurysm or obstructive plaque, for example, can be bypassed during repair, essentially restricting any potentially harmful effects on the body as a whole as might result from requirements of the procedure to the treatment site. An aneurysm at less than operable size or one recently repaired, for example, can be bypassed with the blood returned to the artery past the vulnerable segment through a return jacket. In open repair, unless some circumstance becomes evident to recommend that the jackets and line or lines be left indefinitely as a prosthesis, these are removed prior to closing. That the foam lining the side-entry jackets allow for growth, and the accessory channels allow for the direct targeting of drugs, closure with a small child is possible.

The applications of side-entry and valve or diversion jackets should not be interpreted in a limiting sense. For example, vascular bypass using the means to be described is not limited to the carotids or to atherosclerotic plaque, fistulae, aneurysms, false aneurysms, and dissections in the vasculature can arise for any of several reasons, some more durably corrected with the means described herein (see, for example, Bansal, N. 2017. “Evolution of Cardiovascular Disease During the Transition to End-Stage Renal Disease,” Seminars in Nephrology 37(2): 120-131; Grams, M. E., Yang, W., Rebholz, C. M., Wang, X., Porter, A. C., and 10 others 2017. “Risks of Adverse Events in Advanced CKD [chronic kidney disease]: The Chronic Renal Insufficiency Cohort (CRIC) Study,” American Journal of Kidney Diseases pii: S0272-S6386(17) 30543-30547; Shiu, Y. T., Litovsky, S. H., Cheung, A. K., Pike, D. B., Tey, J. C., and 5 others 2016. “Preoperative Vascular Medial Fibrosis and Arteriovenous Fistula Development,” Clinical Journal of the American Society of Nephrology 11(9): 1615-1623; Fatemifar, F. and Han, H.-C. 2016. “Effect of Axial Stretch on Lumen Collapse of Arteries,” Journal of Biomechanical Engineering 138(12): 1245031-1245036; Gilbert, R, E. 2013. “Endothelial Loss and Repair in the Vascular Complications of Diabetes: Pathogenetic Mechanisms and Therapeutic Implications,” Circulation Journal 77(4): 849-856; Zeadin, M. G., Petlura, C. I., and Werstuck, G, H. 2013. “Molecular Mechanisms Linking Diabetes to the Accelerated Development of Atherosclerosis,” Canadian Journal of Diabetes 37(5): 345-350; Nasser, M., Vegal. M. B., Pivetta, L, G. A., Nasser, A. I., and Melo, D, G. 2013. “Internal Carotid Artery Dissection in a Patient with Ehlers-Danlos Syndrome Type I V: Diagnosis and Management,” Jornal Vascular Brasileiro 12(2); Puri, R., Kataoka, Y., Uno, K., and Nicholls, S. 2012. “The Distinctive Nature of Atherosclerotic Vascular Disease in Diabetes: Pathophysiological and Morphological Insights,” Current Diabetes Reports 12(3): 280-285; Greif, D. M., Kumar, M., Lighthouse, J. K., Hum, J., An, A., Ding, L., Red-Horse, K., and 4 others 2012. “Radial Construction of an Arterial Wall,” Developmental Cell S23(3): 482-493; Lum, Y. W., Brooke, B. S., and Black, J, H. 3rd. 2011. “Contemporary Management of Vascular Ehlers-Danlos Syndrome,” Current Opinion in Cardiology 26(6): 494-501; Hodis, S. and Zamir M. 2009. “Mechanical Events Within the Arterial Wall: The Dynamic Context for Elastin Fatigue,” Journal of Biomechanics 42(8): 1010-1016; Spinetti, G., Kraenkel, N., Emanueli, C., and Madeddu, P. 2008. “Diabetes and Vessel Wall Remodelling: From Mechanistic Insights to Regenerative Therapies,” Online, Cardiovascular Research (Oxford, England) 78(2): 265-273).

Diversion or takeoff from the artery is through the jacket, bypass catheter, and downstream return side-entry or side-entry diversion jacket, thus avoiding the need for direct synthetic to tissue anastomoses. Thus, neither type jacket is end-to-end anastomosed to the artery at either end but rather placed in collaring or mantling relation to the substrate artery, eliminating suture lines and the complications these bode. The internal moisture vapor protected viscoelastic polyurethane lining the jacket is less likely to provoke an adverse tissue reaction, and should any arise, anti-inflammatory medication can be delivered into the synthetic-tissue interface along with antimicrobials and any other medication in fluid form.

Weaknesses in the vasculature that are highly susceptible to failure are more dependably repaired with side-entry diversion and side-entry jackets used to bypass the weak points. A circumscribed weakness in the vasculature that is highly susceptible to failure if not repeated failure is more dependably negotiated by replacing the segment with a synthetic bypass not susceptible to the weakening conditions rather than through surgical repair. Because the accessory channels allow establishing an anticoagulant drip and the targeted delivery of drugs into it, such a bypass can remain a permanent implant wherewith the drugs delivered are targeted and thus confined to the treatment site, meaning the bypass and the tissue at its origin and destination.

While the diversion of arterial blood is usually from a larger artery to a smaller artery supplying a different territory, another situation where continuous control over the differential division of flow between an artery and a bypass or shunt justifies placement of a valve-jacket is where there is the need to divert blood through a catheter not into its supply artery but rather in emulation of the Vineberg technique, directly into hypoxic tissue, such as a venous stasis ulcers as shown in U.S. Pat. No. 11,759,186, FIGS. 17 thru 19 (see, for example, Bolotin, G., Medalion, B., Ghandakly, E. C., lacona, G. M., and Bakaeen, F, G. 2024. “Coronary Artery Surgery: Past, Present, and Future,” Online, Rambam Maimonides Medical Journal (Haifa, Israel) 15(1): e0001; Mack, M. J., Squiers. J. J., Lytle, B. W., DiMaio, J. M., and Mohr, F. W. 2021. “Myocardial Revascularization Surgery: JACC Historical Breakthroughs in Perspective,” Online, Journal of the American College of Cardiology (New York, New York) 78(4): 365-383; Diodato, M. and Chedrawy, E, G. 2014. “Coronary Artery Bypass Graft Surgery: The Past, Present, and Future of Myocardial Revascularisation,” Online, Surgery Research and Practice (New York, New York) 2014:726158; Buxton, B, F. and Galvin, S, D. 2013. “The History of Arterial Revascularization: From Kolesov to Tector and Beyond” Annals of Cardiothoracic Surgery 2(4): 419-426; Katrapati, P. and George, J, C. 2008. “Vineberg Operation: A Review of the Birth and Impact of this Surgical Technique,” Annals of Thoracic Surgery 86(5): 1713-1716; Katrapati, P. and George, J, C. 2008. “Vineberg Operation: A Successful Case 35 Years Later,” Annals of Thoracic Surgery 86(5): 1676-1677; Thomas, J. L. 1999. “The Vineberg Legacy: Internal Mammary Artery Implantation from Inception to Obsolescence,” Texas Heart Institute Journal 26(2): 107-113; Katrapati, P. and George, J, C. 2008. “Vineberg Operation: A Review of the Birth and Impact of this Surgical Technique,” Annals of Thoracic Surgery 86(5): 1713-1716; Shrager, J, B. 1994. “The Vineberg Procedure: The Immediate Forerunner of Coronary Artery Bypass Grafting,” Annals of Thoracic Surgery 57(5): 1354-1364) or more likely (and retracing the history of the Vineberg approach, except using synthetic catheters rather than the middle thoracic as the conduits), into an artery supplying the supply territory or zone of the smaller artery. The use of nonjacketing side-entry connectors for this purpose is described and illustrated in U.S. Pat. No. 11,013,858, FIGS. 17 thru 19.

If a slight increase in pressure is desired through the native vessel, a local, or topical vasoconstrictor (pressor, vasopressor) such as suitably formulated in fluid, and if necessary, post-liver passed form, caffeine, epinephrine, tetryzoline, angiotensin II, a vasopressin, phenoxybenzamine, oxymetazoline hydrochloride, xylometazoline, dopamine, dobutamine, phenylephrine, nicotine, acetaminophen, or paracetamol, or desglymidodrine, is released through a side-entry jacket drugline to pass over the treatment site without dependency upon the kidneys, liver, or heart and thus without significant effect on the systemic or pulmonary pressures.

Vasoconstrictors such as higher levels of circulating epinephrinere frequently discussed in the context of anesthetic adjuvants (see, for example, Dalal, R. and Grujic, D. 2024. “Epinephrine,” Online, Treasure Island Florida: StatPearls Publishing Co.; Zhou, X., Zhong, Y., Pan, Z., Zhang, J., and Pan, J. 2023. “Physiology of Pregnancy and Oral Local Anesthesia Considerations,” Online, PeerJ (Corte Madera, California) 11: e15585; Singla, M., Gugnani, M., Grewal, M. S., Kumar, U., and Aggarwal. V. 2022. “Does the Presence and Amount of Epinephrine in 2% Lidocaine Affect Its Anesthetic Efficacy in the Management of Symptomatic Maxillary Molars with Irreversible Pulpitis?,” Online, Journal of Dental Anesthesia and Pain Medicine (Seoul, South Korea) 22(1): 39-47; Tusman, G., Acosta, C. M., Pulletz S. Bohm S H, Scandurra A, and 3 others 2019. “Photoplethysmographic Characterization of Vascular Tone Mediated Changes in Arterial Pressure: An Observational Study,” Journal of Clinical Monitoring and Computing (Amsterdam, North Holland, Netherlands) 33(5): 815-824; Naftalin, L. W. and Yagiela, J, A. 2002. “Vasoconstrictors: Indications and Precautions,” Dental Clinics of North America (Philadelphia, Pennsylvania) 46(4): 733-746, ix; Yagiela, J, A. 1995. “Vasoconstrictor Agents for Local Anesthesia,”. Online, Anesthesia Progress (Lawrence, Kansas) 42(3-4): 116-120; Sisk, A. L. 1992. “Vasoconstrictors in Local Anesthesia for Dentistry,” Online, Anesthesia Progress (Lawrence, Kansas) 39(6): 187-193; Frelin, C. 1991. “Mechanisms of Vasoconstriction,” American Heart Journal (St. Louis, Missouri) 121(3 Part 1): 958-960; Lindorf, H. H. 1979. “Investigation of the Vascular Effect of Newer Local Anesthetics and Vasoconstrictors,” Oral surgery, Oral medicine, and Oral pathology (St. Louis, Missouri) 48(4): 292-297).

Vasodilators to lower local blood pressure can be released by direct feed into the vessel through a side-entry jacket or vascular valve drugline. Local vasodilators such as papaverine, lidocaine, phosphodiesterase inhibitors, nifedipine, verapamil, and calcium channel blockers are often local anesthetics (see, for example, Ueda, M., Hirayama, Y., Ogawa, H., Nomura, T., Terashi, H., and Sakakibara, S. 2023. “Vasodilating Effects of Antispasmodic Agents and Their Cytotoxicity in Vascular Smooth Muscle Cells and Endothelial Cells-Potential Application in Microsurgery,” Online, MDPI [Multidisciplinary Digital Publishing Institute] International Journal of Molecular Sciences (Basel, Switzerland) 24(13): 10850; Rinkinen, J. and Halvorson, E, G. 2017. “Topical Vasodilators in Microsurgery: What is the Evidence?,” Journal of Reconstructive Microsurgery (New York, New York) 33(1): 1-7; Vargas, C. R., Iorio, M, L., and Lee, B. T. 2015. “A Systematic Review of Topical Vasodilators for the Treatment of Intraoperative Vasospasm in Reconstructive Microsurgery,” Plastic and Reconstructive Surgery (Hagerstown, Maryland) 136(2): 411-422; Yu, J. T., Patel, A. J., and Malata, C. M. 2011. “The Use of Topical Vasodilators in Microvascular Surgery,” Journal of Plastic, Reconstructive and Aesthetic Surgery (Amsterdam, North Holland) 64(2): 226-228).

Where drugs would best be avoided, the blood pressure can be controllably and precisely elevated through this circumscribed segment through the placement of an outlet vascular servochoke of servovalve rather than a bistable solenoid-driven chute valve, outflow prevented by clamping the sideline. The choke will increase the upstream pressure while the servovalve will generally serve to gate the input into a shunt controlled by the system microcontroller, or in multimorbid disease, the master control microprocessor. The drugless lowering of the local blood pressure can be accomplished with a tuned shunt as addressed below in sections 2b(2)(h), entitled Local Vascular Pressure Relief, its subsidiary sections, and FIG. 22F showing a tuned shunt. Positioning the servochoke at the inlet allows increasing the blood pressure upstream to it while lowering the pressure downstream to the vessel entered.

The jacket also makes possible the direct targeting of both the pressure drop and medication to facilitate healing rather than to adjust the blood pressure, advantageous where the drugs when combined pose problems. Following the procedure, the pressure can be controllably reduced at the treatment site by drawing off (diverting, tapping off) a portion of the flow and systemically by continuing the release of dexmedetomidine.

Placement of separate adjustable jackets just inferior to the segment to be treated or a single jacket with antipodal chutes each able to divert half of the flow through the common carotid to either of two return or reentry jackets, one each on the internal and the other on the external carotid, each providing half diversion away from the operated segment through their catheters respectively connected to the inferior to the superior jackets, together diverting all flow, allows diverting a controllable fraction of the normal flow through the artery with retum through a downstream jacket past the repair.

A jacket with continuously deployable chute constitutes a servovalve controlled in-line valve, which placed in the vascular tree with chute fully deployed or extended to fully obturate the native lumen, can be used to temporarily or permanently divert all flow through the native lumen into a shunt, leading to an extracorporeal hemodialysis or apheresis machine, for example, or into a bypass, and when attached to a ureter, can shunt the flow of urine into a paracorporeal collection pouch or a bypass about a missing or healing segment of the ureter, the urine then diverted into the bladder if unimpaired.

In a patient too elderly for the successive clonal steps in the development of malignancy to materialize (Morin, P. J., Trent, J. M., Collins, F. S., and Vogelstein, B. 2005. “Cancer Genetics,” in Kasper, D. L, Braunwald, E., Fauci, A. S., Hauser, S, L., Longo, D, L., and Jameson, J. L. Harrison's Principles of Internal Medicine, Chapter 68, page2 447-448, New York, New York: McGraw-Hill Medical Publishing Division) following exposure to ionizing radiation of a dose rate sufficient to eventuate before the patient would die due to some other cause, and in whom this risk is judged less a threat than that primary, the avoidance of transmitting higher dose rate ionizing radiation through the radiation sensitive intimae can be relaxed. Since the exposure is not confined to the intima inside the jacket or connector, the need for radiation shielding beneath the outer shell offers little if any protective benefit.

This however, should be studiously avoided in the young, since these successive clonal steps in the development of malignancy could well be initiated by early exposure to radiation of this intensity—“Exposure to ionizing radiation at an earlier age appears to increase the chance of developing radiation-induced carcinomas,” (Hahn, S. M. and Glatstein, E. 2005. “Principles of Radiation Therapy,” Chapter 71, page 488, in Kasper, D. L, Braunwald, E., Fauci, A. S., Hauser, S, L., Longo, D, L., and Jameson, J. L. Harrison's Principles of Internal Medicine, Chapter 68, page2 447- 448, New York, New York: McGraw-Hill Medical Publishing Division).

In more complex applications where sensors in the jacket or implanted elsewhere in the body detect the need to release various drugs into one or more ductus at different times, control over the in-line valve or valves is entrusted to an implanted microcontroller governed by a prescription-program or a clinical remote-control center in radio communication with the jacket such as through a cellular telephone. Such automated and adaptive control is described in U.S. Pat. No. 11,759,186, entitled Ductus Side-entry Jackets and Prosthetic Disorder Response Systems. Regulated by an automatic control system, an in-line valve along the vascular tree is a medically adapted electrical servovalve and one along a ureter a medically adapted kind of electrohydraulic servovalve.

Fractional directly targeted use with a background systemic dose as appropriate includes, for example, positioning the jacket upstream to a malignant solid tumor, for example, thus adding mechanical to chemical or metabolic targeting of chemotherapeutics, which might be nanoparticulate, supraparamagnetic, or superparamagnetic carrier-bonded nanoparticulate, and/or insulin potentiated (see, for example, Li, B., Li, Q., Mo, J., and Dai, H. 2017. “Drug-loaded Polymeric Nanoparticles for Cancer Stem Cell Targeting,” Online, Frontiers in Pharmacology 8:51; Gong, M. Q., Wu, C., He, X. Y., Zong, J. Y., Wu, J, L., Zhuo, R. X., and Cheng, S. X. 2017. “Tumor Targeting Synergistic Drug Delivery by Self-Assembled Hybrid Nanovesicles to Overcome Drug Resistance,” Pharmaceutical Research 34(1): 148-160; Goh, W. J., Lee, C. K., Zou, S., Woon, E. C., Czarny, B., and Pastorin, G. 2017. “Doxorubicin-loaded Cell-derived Nanovesicles: An Alternative Targeted Approach for Anti-tumor Therapy,” International Journal of Nanomedicine 12:2759-2767; Lu, B., Huang, X., Mo, J., and Zhao, W. 2016. “Drug Delivery Using Nanoparticles for Cancer Stem-Like Cell Targeting,” Online, Frontiers in Pharmacology 7:84; Klement, R. J. and Fink, M. K. 2016. “Dietary and Pharmacological Modification of the Insulin/IGF-1 System: Exploiting the Full Repertoire Against Cancer,” Oncogenesis 5: e193; Perez-Herrero, E. and Fernandez-Medarde, A. 2015. “Advanced Targeted Therapies in Cancer: Drug Nanocarriers, Future of Chemotherapy,” European Journal of Pharmaceuties and Biopharmaceuties 93:52-79; Liu, B., Ezeogu, L., Zellmer, L., Yu, B., Xu, N., Joshua Liao, D. 2015. “Protecting the Normal in Order to Better Kill the Cancer,” Cancer Medicine 4(9): 1394-1403; Damyanov, C., Gerasimova, D., Maslev, I., and Gavrilov, V. 2012. “Low-dose Chemotherapy with Insulin (Insulin Potentiation Therapy) in Combination with Hormone Therapy for Treatment of Castration-resistant Prostate Cancer,” ISRN [International Scholarly Research Network] Urology 2012:140182; Damyanov, C., Radoslavova, M., Gavrilov, V., and Stoeva, D. 2009. “Low Dose Chemotherapy in Combination with Insulin for the Treatment of Advanced Tumors. Preliminary Experience,” Journal of the BUON [Balkan Union of Oncology] 14(4): 711-715).

Uptake of the drug or drugs over a delimited segment of the target ductus is obtained by injecting or infusing the drug or drugs through the surface port in the form of a ferrofluid wherein these are rendered magnetically susceptible by means of carrier bonding. As described in U.S. Pat. No. 11,013,858, entitled Nonjacketing Side-entry Connectors and Prosthetic Disorder Response Systems, and in U.S. Pat. No. 11,389,171 entitled Integrated System for the Infixion and Retrieval of Implants, uptake is then by a jacket comprised of or incorporating one or more permanent or electromagnets.

If the injectant or infusate is radioactive, the port, accessory channel, and nonjacketing side-entry connector are radiation shielded. Successive neodymium magnets in the downstream, or antegrade, direction are generally increased in magnetic strength as a gradient array, and electromagnets adjusted in field strength in like manner, the exact field strength in either case depending upon the flow rate and pressure of the blood or urine passing the jacket through the substrate ductus.

Radiation shielding treatment of drug reservoirs, reservoir outlet pumps or drip stopcocks, druglines, and nonjacketing side-entry connectors is shown in FIGS. 9, 10, and 10A of U.S. Pat. No. 11,013,858, entitled Nonjacketing Side-entry Connectors and Prosthetic Disorder Response Systems, delineating radiation shielding, absorbable and permanent, at length. In making possible the direct pipeline targeting of radioactive chemotherapeutics to nidi, such components open the way for a new oncology.

As might any sufficiently miniaturized probe or sensor, one or more thin film piesoresistance-based strain gauge type pressure sensors can be incorporated into the diversion jacket or valve or positioned along the internal surface of the synthetic bypass or conduit. For the purpose stated, if the pressure is normal, the nominal pressure range can be used; if the pressure is abnormal, accurate measurement is necessary (see, for example, Ghafoori Yazdi, M., Shoghli, A., Faghihi, S., and Baratloo, A. 2016. “Central Venous Pressure Monitoring; Introduction of a New Device,” Emergency (Tehran) 4(2): 52-54; Yannoutsos, A., Rinaldi, E. R., Zhang, Y., Protogerou, A. D., Safar, M. E., and Blacher, J. 2015. “Central Hemodynamics in Risk Assessment Strategies: Additive Value Over and Above Brachial Blood Pressure,” Current Pharmaceutical Design 21(6): 719-729; Rinaldi, E. R., Yannoutsos, A., Borghi, C., Safar, M. E., and Blacher, J. 2015. Central Hemodynamics for Risk Reduction Strategies: Additive Value Over and Above Brachial Blood Pressure,” Current Pharmaceutical Design 21(6): 730-736; McEniery, C. M., Cockcroft, J. R., Roman, M. J., Franklin, S. S., and Wilkinson, I, B. 2014. “Central Blood Pressure: Current Evidence and Clinical Importance,” European Heart Journal 35(26): 1719-1725; Miyashita, H. 2012. “Clinical Assessment of Central Blood Pressure,” Current Hypertension Reviews 8(2): 80-90). The use of side-entry and side-entry diversion jackets allows more safe and secure ambulatory monitoring (Garcia-Ortiz L I, Ramos-Delgado E, Recio-Rodriguez J I, Agudo-Conde C, Martinez-Salgado C, and 3 others 2011. “Peripheral and Central Arterial Pressure and Its Relationship to Vascular Target Organ Damage in Carotid Artery, Retina and Arterial Stiffness. Development and Validation of a Tool. The Vaso Risk Study,” Online, BioMed Central Public Health 11:266).

For example, methotrexate therapy of a malignant tumor consists of positioning the jacket upstream along the blood supply to the location of the tumor as illustrated in U.S. Pat. No. 11,759,186, entitled Ductus Side-entry Jackets and Prosthetic Disorder Response Systems, FIGS. 16 and 21. If necessary, uptake along the ensuing segment is expedited by magnet attraction of the drug delivered in a ferrofluid. If the drug is not to be allowed into or added to that already in the circulation, a second jacket at the distal end of the segment, likewise assigned a specific opening in the small port at the body surface, is used to release a reversal agent.

Common reversal agents for methotrexate are glucarpidase, the recombinant bacterial enzyme that hydrolyzes the carboxyl terminal glutamate residue from folic acid and classical antifolates such as methotrexate (see, for example, Cavone, J, L., Yang, D., and Wang, A. 2014. “Glucarpidase Intervention for Delayed Methotrexate Clearance,” Annals of Pharmacotherapy 48(7): 897-907; Fermiano, M., Bergsbaken, J., and Kolesar, J. M. 2014. “Glucarpidase for the Management of Elevated Methotrexate Levels in Patients with Impaired Renal Function,” American Journal of Health-system Pharmacy 71(10): 793-798; Rattu, M. A, Shah, N., Lee, J. M., Pham, A. Q., and Marzella, N. 2013. “Glucarpidase (Voraxaze), a Carboxypeptidase Enzyme for Methotrexate Toxicity,” Pharmacy and Therapeutics 38(12): 732-744) and especially for overdoses, the reduced folate leucovorin (5-formyltetrahydrofolate, folinic acid) (see, for example, Boarman, D. M., Baram, J., and Allegra, C. J. 1990. “Mechanism of Leucovorin Reversal of Methotrexate Cytotoxicity in Human MCF-7 [Michigan Cancer Foundation-7] Breast Cancer Cells,” Biochemical Pharmacology 40(12): 2651-2660; Kennedy, D. G., Van den Berg, H. W., Clarke, R., and Murphy, R, F. 1985. “The Effect of Leucovorin on the Synthesis of Methotrexate Poly-gamma-glutamates in the MCF-7 Human Breast Cancer Cell Line,” Biochemical Pharmacology 34(16): 2897-2903).

When not directly targeted at the tumor thus, methotrexate in the circulation is eliminated with a reversal agent (Cavone, J, L., Yang, D., and Wang, A. 2014. Op cit.) or reversal agents combined with a nonmedicinal process (Connors, N. J, Sise, M. E., Nelson, L. S., Hoffman, R. S., and Smith, S. W. 2014. “Methotrexate Toxicity Treated with Continuous Venovenous Hemofiltration, Leucovorin and Glucarpidase,” Clinical Kidney Journal 7(6): 590-592).

In the treatment of a malignant tumor with a tendency to metastasize, a background systemic dose is used to supplement, and along with radiation if necessary, assist in eradicating the shedding of malignant ‘daughter’ cells. A nonjacketing side-entry connector is attached directly above or to penetrate the tumor in the parenchyma with drug delivery or therapeutic tool within or close to the tumor. Such an application with proximate drug delivery is illustrated U.S. Pat. No. 11,013,858, Nonjacketing Side-entry Connectors and Prosthetic Disorder Response Systems FIG. 6.

Treatment with drug and/or other therapy into the tumor itself through a nonjacketing side-entry connector fastened at the tumor is illustrated in FIGS. 13A and 13B, which can also include or take the form direct treatment with a miniature laser, electrode, injection needle, or any other styloid therapeutic tool at the distal end of a miniature cabled device. Moreover, with radiation shielding, contrast, tracers, and drugs directly targeted thus can be radioactive.

The targeted fraction seldom requires a downstream jacket to deliver a reversal agent, and less still when systemic dispersal is necessary. The permanent implantation of catheters is made possible primarily by the ductus side-entry jacket accessory or service channel or channels, which allow the direct targeting to the jacket and line of crystallization, coagulation, and biofilm formation counteractants to eliminate the occlusion that had prevented the use of smaller gauge synthetic tubing for such purposes. Significantly, the placement of any side-entry jacket except one that requires the cooperation of magnetically susceptible matter in the wall of the ductus used to stent the ductus, as described in U.S. Pat. No. 11,389,171, entitled Integrated System for the Infixion and Retrieval of Implants, involves no endoluminal entry.

Since no part of the distal end of the catheter is introduced into the vascular lumen, thrombophilia-inducing turbulence, augmented and compounded with further complications due to the presence of a foreign body in the lumen, to include stenosis and the fibrous encapsulation of a long-term chemotherapy or hemodialysis catheter, for example, which the use of side-entry and side-entry diversion jackets, expressly intended for long-term use, should eliminate (see, for example, Adeel Ebad, C., Davitt, S., Gnanasekaran, R., Khan, A., and Moran, A. M. 2016. “Application of Hong's Technique for Removal of Stuck Hemodialysis Tunneled Catheter to Pacemaker Leads,” Radiology Case Reports 12(1): 97-101; Vellanki, V. S., Watson, D., Rajan, D. K., Bhola, C. B., and Lok, C, E. 2015. “The Stuck Catheter: A Hazardous Twist to the Meaning of Permanent Catheters,” Journal of Vascular Access (Thousand Oaks, California) 16(4): 289-293), and neither should the need to remove the catheter mid-treatment arise (see, for example, Jaberi, A., Hadziomerovic, A., Toor, S. S., Galwa, R. P., Graham, J., Thornhill, R. E., and Ryan, S, E. 2014. “Externalization of Tunneled Hemodialysis Catheter in Patients with Tunnel or Exit-site Infections and Limited Access Options,” Journal of Vascular and Interventional Radiology 25(4): 561-566; further references below). Long recognized, the use of catheters to convey blood has been prevented by eventual occlusion by thrombus not warranting the high dose administration of a systemic anticoagulant. The deposition of crystal along the internal walls of urinary stents means that these must be replaced every three months.

A central feature of side-entry connectors and jackets, of which a flow diversion jacket is an adaptation that incorporates this feature and can incorporate any of the other features of conventional connectors and jacket, is a subsidiary side-entry line referred to as an accessory channel or sideline that allows drugs and other agents to be delivered directly to the substrate tissue underlying a connector and wet down the native or catheteric lumen and tissue to which a jacket is attached. Migration a significant problem, all side-entry jackets have suture loops or eyelets on the outer shell to through which to pass suture and thus secure the jacket at the level where it is placed. This allows fixing an inferior vena cava filter or another side-entry jacket in position. Side-entry jackets, diversion jackets, and nonjacketing side-entry connectors can incorporate diagnostic, therapeutic, and viewing sensors, probes incorporating sensors, or connected to sensors positioned outside the jacket or connector.

Invulnerable to disease, shear stress, initiating a cytokine cascade upon being injured, endothelial breakdown when compressed, stenosis, or turbulent flow (see, for example, Simmons, R. D., Kumar, S., and Jo, H. 2016. “The Role of Endothelial Mechanosensitive Genes in Atherosclerosis and Omics Approaches,” Archives of Biochemistry and Biophysics 591: 111-131; Simmons, R. D., Kumar, S., Thabet, S. R., Sur, S., and Jo, H. 2016. “Omics-based Approaches to Understand Mechanosensitive Endothelial Biology and Atherosclerosis,” Wiley Interdisciplinary Reviews. Systems Biology and Medicine 8(5): 378-401; Moonen, J. R., Lee, E. S., Schmidt, M., Maleszewska, M., Koerts, J. A., Brouwer, L. A., van Kooten, T. G., and 4 others 2015. “Endothelial-to-Mesenchymal Transition Contributes to Fibro-proliferative Vascular Disease and is Modulated by Fluid Shear Stress,” Cardiovascular Research 108(3): 377-386; further references below), the preferability of using smaller gauge catheters rather than harvested vessels has long been obvious but obstructed by two major obstacles.

The first is that if not counteracted, thrombus (clot) and/or biofilm in the bloodstream, while in the urinary tract, crystal deposits may accumulate along the internal walls of the tubing to reduce the luminal diameter. As addressed below, the other is that the interface between the native and synthetic components is subject to degradation as a foreign body. Ductus, to include diversion, side-entry jackets and nonjacketing side-entry connectors alleviate the deposition of thrombus along the internal walls of vessels and crystal along the internal walls of the ureters, for example, by providing one or more collateral passageways, or accessory channels, into the side-entry jacket or connector and native ductus to which the side-entry device is connected for the delivery of remediating agents-anticoagulants in vessels and crystal solvents in the ureters, with antimicrobial, anti-inflammatoires, and an anesthetic, for example.

At the same time, adverse tissue reactions that would induce tissue breakdown and irritation are minimized if not eliminated by avoiding direct-contact native ductus end-to-end or end-to-side anastomosis, by embedding the foam Parylene® (Diamond-MT [owner initials]. Johnstown, Pennsylvania) or a similar coating film lining with a nanoparticulate antibiotic, and as addressed above in this section, exceptionally, by using an accessory channel to target adverse reaction-remediating medication into the jacket foam lining-native adventitia interface. Apposition of native tissue and synthetic materials are thus made substantially safe and secure.

Generally, a conventional side-entry jacket with surface port is used to target drugs or parenteral nutrition, for example, directly into the vascular tree, whereas a diversion side-entry jacket is used to partially or completely shunt away the stream for processing through a dialysis apheresis, or plasmapheresis machine, for example. Where anastomoses are desired, as in a solid organ transplant in a fetus or neonate to allow growth, the jackets with mainline removed and side connector capped off and accessory channels are left in place to target medication from the surface port to the anastomoses.

Thrombosis, especially in patients with a malignancy, any other condition that induces thrombophilia, sickle cell, and other diseases, along with infection, has long been recognized as a major complication associated with the use of vascular access lines (see, for example, Carlon, T, A. and Sudheendra, D. 2017. “Interventional Therapy for Upper Extremity Deep Vein Thrombosis,” Seminars in Interventional Radiology 34(1): 54-60; McGill, R, L., Ruthazer, R., Meyer, K. B., Miskulin, D. C., and Weiner, D, E. 2016. “Peripherally Inserted Central Catheters and Hemodialysis Outcomes,” Clinical Journal of the American Society of Nephrology 11(8): 1434-1440; Zhang, X., Huang, J. J., Xia, Y., Li, C. F., Wang, Y., Liu, P. P., Bi. X. W., and 4 others 2016. “High Risk of Deep Vein Thrombosis Associated with Peripherally Inserted Central Catheters in Lymphoma,” Oncotarget 7(23): 35404-35411; Refaei, M., Fernandes, B., Brandwein, J., Goodyear, M. D., Pokhrel. A., and Wu, C. 2016. “Incidence of Catheter-related Thrombosis in Acute Leukemia Patients: A Comparative, Retrospective Study of the Safety of Peripherally Inserted vs. Centrally Inserted Central Venous Catheters,” Annals of Hematology 95(12): 2057-2064; Menendez, J. J., Verdu, C., Calderon, B., Gomez-Zamora, A., Schuffelmann, C., de la Cruz, J. J., and de la Oliva, P. 2016. “Incidence and Risk Factors of Superficial and Deep Vein Thrombosis Associated with Peripherally Inserted Central Catheters in Children,” Journal of Thrombosis and Haemostasis 14(11): 2158-2168; Kalloo, S. and Wish, J, B. 2016. “Nephrologists Versus Peripherally Inserted Central Catheters: Are the PICCs Winning?,” Clinical Journal of the American Society of Nephrology 11(8): 1333-1334; Fallouh, N., McGuirk, H. M., Flanders, S. A., and Chopra, V. 2015. “Peripherally Inserted Central Catheter-associated Deep Vein Thrombosis: A Narrative Review,” American Journal of Medicine 128(7): 722-738; Sereno, M., Montoro, F. J., Casanova, C., Gutierrez-Gutierrez, G., Ojeda, J., and Casado, E. S. 2015. “An Unusual Cause of Respiratory Failure in a Colon Cancer Patient,” Molecular and Clinical Oncology 3(5): 1152-1154; Austin, R. E., Shahrokhi, S., Bolourani, S., and Jeschke, M, G. 2015. “Peripherally Inserted Central Venous Catheter Safety in Burn Care: A Single-center Retrospective Cohort Review,” Journal of Burn Care and Research 36(1): 111-117; Zochios, V., Umar, I., Simpson, N., and Jones, N. 2014. “Peripherally Inserted Central Catheter (PICC)-related Thrombosis in Critically Ill Patients,” Journal of Vascular Access (Thousand Oaks, California) 15(5): 329-337; Cotogni, P. and Pittiruti, M. 2014. “Focus on Peripherally Inserted Central Catheters in Critically Ill Patients,” World Journal of Critical Care Medicine 2014 3(4): 80-94; Pan, L., Zhao, Q., and Yang, X. 2014. “Risk Factors for Venous Thrombosis Associated with Peripherally Inserted Central Venous Catheters,” International Journal of Clinical and Experimental Medicine 2014 7(12): 5814-5819; Chopra, V., Anand, S., Hickner, A., Buist, M., Rogers, M. A., Saint, S., and Flanders, S, A. 2013. “Risk of Venous Thromboembolism Associated with Peripherally Inserted Central Catheters: A Systematic Review and Meta-analysis,” Lancet 382(9889): 311-325; Naik, R. P., Streiff. M. B., and Lanzkron, S. 2013. Sickle Cell Disease and Venous Thromboembolism: What the Anticoagulation ExpNert Needs to Know,” Journal of Thrombosis and Thrombolysis 35(3): 352-358; Marnejon, T., Angelo, D., Abu Abdou, A., and Gemmel, D. 2012. “Risk Factors for Upper Extremity Venous Thrombosis Associated with Peripherally Inserted Central Venous Catheters,” Journal of Vascular Access (Thousand Oaks, California) 13(2): 231-238; Lordick, F., Hentrich, M., Decker, T., Hennig, M., Pohlmann, H., Hartenstein, R., and Peschel, C. 2003. “Ultrasound Screening for Internal Jugular Vein Thrombosis Aids the Detection of Central Venous Catheter-related Infections in Patients with Haemato-oncological Diseases: A Prospective Observational Study,” British Journal of Haematology 120(6): 1073-1078), and especially so in patients already prone to clotting such as those with cancer (references below).

The ports for use with vascular access devices to be described fit flush and stably to the upper and/or lower surface of the skin and incorporate an antiseptic chamber below a cap as not to require frequent changes of a dressing and securement adhesive or adhesive tape. These features eliminate the need for a properly applied sterile dressing and securement essential to avoid damaging the skin, increasing the chance of infection and eliminating the need for critical maintenance for the very old or young (see, for example, Takashima, M., Ray-Barruel. G., Ullman, A., Keogh, S., and Rickard, C. M. 2017. “Randomized Controlled Trials in Central Vascular Access Devices: A Scoping Review,” Online, PLoS [Public Library of Science] One 12(3): e0174164; Marsh, N., Webster. J., Mihala, G., and Rickard, C. M. 2017. “Devices and Dressings to Secure Peripheral Venous Catheters: A Cochrane Systematic Review and Meta-analysis,” International Journal of Nursing Studies 67:12-19; Gavin, N. C., Webster, J., Chan, R. J., and Rickard, C. M. 2016. “Frequency of Dressing Changes for Central Venous Access Devices on Catheter-related Infections,” Cochrane Database of Systematic Reviews 2: CD009213; Rickard, C. M., Edwards, M., Spooner, A. J., Mihala, G., Marsh, N., Best. J., Wendt. T., and 5 others 2016. “A 4-arm Randomized Controlled Pilot Trial of Innovative Solutions for Jugular Central Venous Access Device Securement in 221 Cardiac Surgical Patients,” Journal of Critical Care 36:35-42; Ullman, A. J., Kleidon, T., Gibson. V., Long, D. A., Williams, T., McBride, C. A., and 4 others 2016. “Central Venous Access Device SeCurement and Dressing Effectiveness (CASCADE) in Paediatrics: Protocol for Pilot Randomised Controlled Trials,” Online, BMJ [British Medical Journal Publishing Group] Open 6(6): e011197; Lai, N. M., Lai, N. A., O'Riordan, E., Chaiyakunapruk, N., Taylor, J. E., and Tan, K. 2016. “Skin Antisepsis for Reducing Central Venous Catheter-related Infections,” Cochrane Database of Systematic Reviews 7: CD010140; Lai, N. M., Taylor, J. E., Tan, K., Choo, Y. M., Ahmad Kamar, A., and Muhamad, N, A. 2016. “Antimicrobial Dressings for the Prevention of Catheter-related Infections in Newborn Infants with Central Venous Catheters,” Cochrane Database of Systematic Reviews 3: CD011082; Ullman, A. J., Cooke, M, L., Mitchell, M., Lin, F., New. K., Long, D. A., Mihala, G., and Rickard, C. M. 2016. “Dressing and Securement for Central Venous Access Devices (CVADs): A Cochrane Systematic Review,” International Journal of Nursing Studies 59:177-196; Ullman, A. J., Cooke, M, L., Mitchell, M., Lin, F., New, K., Long, D. A., Mihala, G., and Rickard, C. M. 2015. “Dressings and Securement Devices for Central Venous Catheters (CVC),” Cochrane Database of Systematic Reviews(9): CD010367).

The material of the surface port can be impregnated, coated, or bonded with an antimicrobial (antiseptic or antibiotic) (Lai, N. M., Chaiyakunapruk, N., Lai, N. A., O' Riordan, E., Pau, W. S., and Saint. S. 2016. “Catheter Impregnation, Coating or Bonding for Reducing Central Venous Catheter-related Infections in Adults,” Cochrane Database of Systematic Reviews 3: CD007878). The references for protective materials and coatings cited below for use in catheters also apply to the surface ports described herein. When a side-entry diversion jacket is partially opened in the vessel, such as during hemodialysis, when, due primarily to the prospective time of treatment, it is desired that the segment shunted about be kept wetted by blood, the chute protrudes into the lumen; however, the accessory channel running beneath it can deliver an anticoagulant such as low molecular weight heparin into the lumen.

Because delivery is targeted, the dose, whether of the factor XIa inhibitor direct oral anticoagulants apixaban, rivaroxaban, dabigatran, milvexian, the direct factor Xa inhibitor betrixaban, as well as low molecular weight heparin, fondaparinux, or a vitamin K antagonist such as warfarin or the 4-hydroxycoumarin derivative acenocoumarin, or acenocoumarol, for example, is isolated and too small in terms of the patient overall body mass to induce adverse side effects. Such problems imclide bleeding, inadequate coagulation in the event of injury or the need for emergency surgery, ecchymosis, epistaxis, pallor, malaise, dyspepsia, hepatotoxicity, increased risk of stroke upon cessation, or thrombotic microangiopathy as thrombocytopenia if not thrombotic thrombocytopenia purpura and hemolytic anemia throughout the vascular tree. Anticoagulants are also addressed below in section 2b(1)(b)(b6), entitled Heart Cage for the Infrarenal Abdominal Positioning of a Second Heart.

The same applies not just to a segment along the blood supply or drainage of a supplied organ or region of tissue but to the organ or region of tissue itself, where the follow-up jacket or jackets are positioned along one or more major outflow veins. In contrast, connection of the line to the native ductus is completely outside the native lumen, only a small hole in the side of the lumen wall present which inconsequentially if at all disrupts laminal or streamline flow past it. If an adjustable ductus side-entry diversion jacket as described herein, then the jacket diversion chute must be fully retracted into the trepan line ending piece.

The incision or puncture wound to pass through the line into the native lumen and presence therein of a foreign object also initiates a cytokine cascade that induces inflammation and aggravates the tendency to clot, which, for any substance having a reversal agent, a supplementary side-entry jacket distal to that used to deliver the drug or drugs can neutralize by direct targeting into the native lumen of that agent. Atherosclerotic degeneration of the substrate intima of arteries and adverse reactions are also eliminated by providing numerous perforations through the outer shell and foam lining of the jacket or connector to prevent endothelial or urothelial degradation due to compression of the fine vessels and nerves entering and leaving the adventitia to serve the entire thickness of the luminal wall and close off the segment enclosed from the interior environment.

U.S. Pat. No. 11,759,186, entitled Ductus Side-entry Jackets and Prosthetic Disorder Response Systems, provides citations to the literature documenting such degradation. For this reason, a Parylene® (Diamond-MT Corporation, Johnstown, Pennsylvania), or a similar coating film must be provided with micropores or larger apertures (see, for example, Arun, M. Z., Üstünes, L., Sevin, G., and Özer, E. 2015. “Effects of Vitamin C Treatment on Collar-induced Intimal Thickening,” Drug Design, Development, and Therapy 9:6461-6473; Kivela, A., Hartikainen, J., and Yla-Herttuala, S. 2012. “Dotted Collar Placed Around Carotid Artery Induces Asymmetric Neointimal Lesion Formation in Rabbits without Intravascular Manipulations,” Online, BioMed Central Cardiovascular Disorders 12:91; Nobécourt, E., Tabet. F., Lambert, G., Puranik, R., Bao, S., Yan, L., Davies, M. J., Brown, B. E., Jenkins, A. J., Dusting, G. J., Bonnet, D. J., Curtiss. L. K., Barter, P. J., and Rye, K, A. 2010. “Nonenzymatic Glycation Impairs the Antiinflammatory Properties of Apolipoprotein A-I,” Arteriosclerosis, Thrombosis, and Vascular Biology 30(4): 766-772; Reel. B., Oktay, G., Ozkal, S., Islekel. H., Ozer, E. and 4 others 2009. “MMP-2 and MMP-9 [matrix metalloproteinases] alteration in Response to Collaring in Rabbits: The Effects of Endothelin Receptor Antagonism,” Journal of Cardiovascular Pharmacology and Therapeutics 2009 14(4): 292-301; Nicholls, S., Dusting, G. J., Cutri, B., Bao, S., Drummond, G. R., Rye, K. A., and Barter, P. J. 2005. “Reconstituted High-density Lipoproteins Inhibit the Acute Pro-oxidant and Proinflammatory Vascular Changes Induced by a Periarterial Collar in Normocholesterolemnic Rabbits,” Circulation 111(12): 1543-1550; Kerry, Z., Yasa, M., Sevin, G., Reel, B., Yetik Anacak, G., and Ozer, A. 2005. “Diverse Effects of Calcium Channel Blockers in the Collar Model,” Acta Cardiologica 60(5): 493-499; Donetti, E., Baetta, R., Comparato, C., Altana, C., Sartore, S., Paoletti, R., Castano, P., Gabbiani, G., and Corsini, A. 2002. “Polymorphonuclear Leukocyte-myocyte Interaction: An Early Event in Collar-induced Rabbit Carotid Intimal Thickening,” Experimental Cell Research 274(2): 197-206; Bruijns, R, H. and Bult, H. 2001. “Effects of Local Cytochalasin D Delivery on Smooth Muscle Cell Migration and on Collar-induced Intimal Hyperplasia in the Rabbit Carotid Artery,” British Journal of Pharmacology 134(3): 473-483; Crauwels, H. M., Herman, A. G., and Bult, H. 2000. “Local Application of Advanced Glycation End Products and Intimal Hyperplasia in the Rabbit Collared Carotid Artery,” Cardiovascular Research 47(1): 173-182; Sozmen, E. Y., Kerry, Z., Uysal, F., Yetik, G., Yasa, M., Ustunes, L., and Onat, T. 2000. “Antioxidant Enzyme Activities and Total Nitrite/Nitrate Levels in the Collar Model. Effect of Nicardipine,” Clinical Chemistry and Laboratory Medicine 38(1): 21-25; Herman, A., Matthys, K., Van Hove, C., Kockx, M., and Bult, H. 1999. “Oxidized Low-density Lipoprotein Enhances Intimal Thickening and Alters Vascular Reactivity,” Verhandelingen Koninklijke Vlaamse Academie voor Geneeskunde van Belgie [Proceedings of the Belgian Royal Academies of Medicine] 61(1): 19-38; Kerry, Z., Yasa, M., Akpinar, R., Sevin, G., Yetik, G., and 5 others 1999. “Effects of Nicardipine on Collar-induced Intimal Thickening and Vascular Reactivity in the Rabbit,” Journal of Pharmacy and Pharmacology 51(4): 441-447; Yasa, M., Kerry, Z., Yetik, G., Sevin, G., Reel. B., and 5 others 1999. “Effects of Treatment with FK409 [(+/−)-(E)-4-ethyl-2-[(E)-hydroxyimino]-5-nitro-3-hexenamide], a Nitric Oxide Donor, on Collar-induced Intimal Thickening and Vascular Reactivity,” European Journal of Pharmacology 374(1): 33-39; Van Put, D. J., Van Osselaer, N., De Meyer, G. R., Andries, L. J., Kockx. M. M., De Clerck, L. S., and Bult, H. 1998. “Role of Polymorphonuclear Leukocytes in Collar-induced Intimal Thickening in the Rabbit Carotid Artery,” Arteriosclerosis, Thrombosis, and Vascular Biology 18(6): 915-921; De Meyer, G. R., Van Put, D. J., Kockx, M. M., Van Schil, P., Bosmans. R., and 4 others 1997. “Possible Mechanisms of Collar-induced Intimal Thickening,” Arteriosclerosis, Thrombosis, and Vascular Biology 17(10): 1924-1930; Arthur, J. F., Yin, Z, L., Young, H. M., and Dusting, G. J. 1997. “Induction of Nitric Oxide Synthase in the Neointima Induced by a Periarterial Collar in Rabbits,” Arteriosclerosis, Thrombosis, Vascular Biology 17(4): 737-740; Baetta, R., Donetti, E., Comparato, C., Calore, M., Rossi, A., Teruzzi. C., Paoletti, R., Fumagalli, R., and Soma. M, R. 1997. “Proapoptotic Effect of Atorvastatin on Stimulated Rabbit Smooth Muscle Cells,” Pharmacological Research 36(2): 115-121; Matthys, K. E., Van Hove, C. E., Kockx. M. M., Andries, L. J., Van Osselaer, N., Herman, A. G., and Bult, H. 1997. “Local Application of LDL [low-density lipoprotein] Promotes Intimal Thickening in the Collared Carotid Artery of the Rabbit,” Arteriosclerosis, Thrombosis, and Vascular Biology 17(11): 2423-2429; Ustunes, L., Yasa, M., Kerry, Z., Ozdemir, N., Berkan, T., Erhan, Y., and Ozer, A. 1996. “Effect of Verapamil on Intimal Thickening and Vascular Reactivity in the Collared Carotid Artery of the Rabbit,” British Journal of Pharmacology 118(7): 1681-1688; Van Put, D. J., Van Hove, C. E., De Meyer, G. R., Wuyts, F., Herman, A. G., and Bult, H. 1995. “Dexamethasone Influences Intimal Thickening and Vascular Reactivity in the Rabbit Collared Carotid Artery,” European Journal of Pharmacology 294(2-3): 753-761; Arthur, J. F., Dusting, G. J., and Woodman, O. L. 1994. “Impaired Vasodilator Function of Nitric Oxide Associated with Developing Neo-intima in Conscious Rabbits,” Journal of Vascular Research 31(4): 187-194; Reckless, J., Fleetwood, G., Tilling, L., Huber, P. A., Marston, S. B., and Pritchard, K. 1994. “Changes in the Caldesmon Isoform Content and Intimal Thickening in the Rabbit Carotid Artery Induced by a Silicone Elastomer Collar,” Arteriosclerosis and Thrombosis 14(11): 1837-1845).

Digressing momentarily, bypass to avoid an injured, diseased, malformed, or operated upon segment in the vascular tree is by positioning an inverted adjustable diversion jacket above the lesion or defect and an adjustable upright jacket below the lesion or defect and connecting these together with a catheter in an additional accessory channel in either jacket. If no blood is to pass through the bypassed segment to wet the interior surface surrounding the lumen, then nonadjustable jackets are used. Medication to treat the bypassed segment is by delivery through the chute through-coursing accessory channel of the upper jacket. Medication to treat the segment below the return jacket is by delivery through the chute-through coursing accessory channel of the lower jacket, and medication to treat the vessel at a level superior to the bypass is through a separate side-entry jacket.

The presence of certain substances, and/or antimicrobial resistant pathogens, and or transplant incompatible cell surface adhesion molecules in the blood not readily and/or sufficiently remedied by conventional means that would prove especially or selectively injurious to an already diseased or recently operated upon segment of a vessel or the organ the vessel or vessels serve, such as the bronchial arteries to treat the lungs or the hepatic portal vein and hepatic arteries to treat the liver, may justify the placement of a bypass created with ductus or diversion side-entry jackets with or without diversion to an extracorporeal apheresis machine or dialyzer to keep the substance away from, while targeting remedial medication such as antimicrobial into the vulnerable segment. (see, for example, Alp, S. and Akova, M. 2017. “Antibacterial Resistance in Patients with Hematopoietic Stem Cell Transplantation,” Mediterranean Journal of Hematology and Infectious Diseases 9(1): e2017002; Yue, C., Kang. Z., Ai, K., Xu. D., Wu. J., and 4 others 2016. “Virus Infection Facilitates the Development of Severe Pneumonia in Transplant Patients with Hematologic Malignancies,” Oncotarget 7(33): 53930-53940; Ibanez-Samaniego, L., Catalina, M. V., Rincon, D., Lo Iacono, O., Fernandez, A, and 4 others 2016. “Liver Support with Albumin Dialysis Reduces Hepatitis C Virus Viremia and Facilitates Antiviral Treatment of Severe Hepatitis C Virus Recurrence after Liver Transplantation,” Therapeutic Apheresis and Dialysis 20(2): 189-196; Hori. T., Onishi, Y., Kamei, H., Kurata, N., Ishigami, M., Ishizu, Y., and Ogura, Y. 2016. Fibrosing Cholestatic Hepatitis C in Post-transplant Adult Recipients of Liver Transplantation,” Annals of Gastroenterology 29(4): 454-459; Gustinetti, G. and Mikulska, M. 2016. “Bloodstream Infections in Neutropenic Cancer Patients: A Practical Update,” Virulence 7(3): 280-297; Tatarelli, P. and Mikulska, M. 2016. “Multidrug-resistant Bacteria in Hematology Patients: Emerging Threats,” Future Microbiology 11:767-780; Park, J, A. 2016. “Diffuse Alveolar Hemorrhage and Recombinant Factor VIIa Treatment in Pediatric Patients,” Korean Journal of Pediatrics 59(3): 105-113; Bose, C. l., Shah, S. V., Karaduta, O. K., and Kaushal, G. P. 2016. “Carbamylated Low-Density Lipoprotein (cLDL)-mediated Induction of Autophagy and Its Role in Endothelial Cell Injury,” Online, PLoS [Public Library of Science] One 11(12): e0165576; Lee, J. H., Kim, S. K., Kim, S. K., Han, S. B., Lee, J. W., and 6 others 2016. “Increase in Antibiotic-Resistant Gram-Negative Bacterial Infections in Febrile Neutropenic Children,” Infection and Chemotherapy 48(3): 181-189; Viscoli, C. 2016. “Bloodstream Infections: The Peak of the Iceberg,” Virulence 7(3): 248-251; Balsorano, P., Romagnoli, S., Evans, S. K., Ricci, Z., and De Gaudio, A, R. 2016. “Urinary Strong Ion Difference as a Marker of Renal Dysfunction. A Retrospective Analysis,” Online, PLoS [Public Library of Science] One 11(6): e0156941; Patel, N., Bichoupan, K., Ku, L., Yalamanchili, R., Harty, A., Gardenier, D., and 19 others 2016. “Hepatic Decompensation/Serious Adverse Events in Post-liver Transplantation Recipients on Sofosbuvir for Recurrent Hepatitis C Virus,” World Journal of Gastroenterology 22(9): 2844-2854; Fagiuoli, S., Ravasio, R., Luca, M. G., Baldan, A., Pecere, S., Vitale, A., and Pasulo. L. 2015. “Management of Hepatitis C Infection Before and after Liver Transplantation,” World Journal of Gastroenterology 21(15): 4447-4456; Balletto, E. and Mikulska, M. 2015. “Bacterial Infections in Hematopoietic Stem Cell Transplant Recipients,” Mediterranean Journal of Hematology and Infectious Diseases 7(1): e2015045; Lim, K. B., Sima, H. R., Fiel, M. I., Khaitova, V., Doucette, J. T. and 11 others 2015. “Utility of the Low- accelerating-dose Regimen in 182 Liver Recipients with Recurrent Hepatitis C Virus,” World Journal of Gastroenterology 21(20): 6236-6245; Moghnieh, R., Estaitieh, N., Mugharbil, A., Jisr. T., Abdallah, D. I., Ziade, F., Sinno, L., and Ibrahim, A. 2015. “Third Generation Cephalosporin Resistant Enterobacteriaceae and Multidrug Resistant Gram-negative Bacteria Causing Bacteremia in Febrile Neutropenia Adult Cancer Patients in Lebanon, Broad Spectrum Antibiotics Use as a Major Risk Factor, and Correlation with Poor Prognosis,” Online, Frontiers in Cellular and Infection Microbiology 5:11; Kuo, K, L., Hung, S. C., Lee. T. S., and Tamg. D, C. 2014. “Iron Sucrose Accelerates Early Atherogenesis by Increasing Superoxide Production and Upregulating Adhesion Molecules in CKD [chronic kidney disease],” Journal of the American Society of Nephrology 25(11): 2596-2606; Montassier, E., Batard, E., Gastinne, T., Potel. G., and de La Cochetière, M. F. 2013. “Recent Changes in Bacteremia in Patients with Cancer: A Systematic Review of Epidemiology and Antibiotic Resistance,” European Journal of Clinical Microbiology and Infectious Diseases 32(7): 841-850; Apostolov, E. O., Ray, D., Savenka, A. V., Shah, S. V., and Basnakian, A, G. 2010. “Chronic Uremia Stimulates LDL [low-density lipoprotein] Carbamylation and Atherosclerosis,” Journal of the American Society of Nephrology 21(11): 1852-1857; Yamada, G., Iino, S., Okuno, T., Omata, M., Kiyosawa, K., Kumada, H., Hayashi, N., and Sakai, T. 2008. “Virological Response in Patients with Hepatitis C Virus Genotype 1b and a High Viral Load: Impact of Peginterferon-alpha-2a Plus Ribavirin Dose Reductions and Host-related Factors,” Clinical Drug Investigation 28(1): 9-16; Afessa, B. and Peters, S, G. 2006. “Major Complications Following Hematopoietic Stem Cell Transplantation,” Seminars in Respiratory and Critical Care Medicine 27(3): 297-309; Massy, Z. A., Ivanovski, O., Nguyen-Khoa. T., Angulo, J., Szumilak. D., and 6 others 2005. “Uremia Accelerates Both Atherosclerosis and Arterial Calcification in Apolipoprotein E Knockout Mice,” Journal of the American Society of Nephrology 16(1): 109-116; Phan, O., Ivanovski, O., Nguyen-Khoa, T., Mothu, N., Angulo, J., Westenfeld, R., and 8 others 2005. “Sevelamer [a phosphate binder] Prevents Uremia-enhanced Atherosclerosis Progression in Apolipoprotein E-deficient Mice,” Circulation 2005 112(18): 2875-2882; Watt, B. E., Proudfoot, A. T., and Vale, J, A. 2004. “Hydrogen Peroxide Poisoning,” Toxicological Reviews 23(1): 51-57; Kellum, J, A. 2000. “Determinants of Blood pH in Health and Disease,” Critical Care (London) 4(1): 6-14).

Unlike use in the urinary tract, where diversion is seldom partial, use in a vessel, usually and artery, diversion will almost always be partial by adjusting the chute to an intermediate extent. Fixed partial diversion in the bloodstream can be obtained with a conventional side-entry jacket with inclined diversion chute and accessory channel used as the excurrent line as shown in U.S. Pat. No. 11,759,186, entitled Ductus Side-entry Jackets and Prosthetic Disorder Response Systems, in FIGS. 21 and 22, or a side-entry vascular servovalve. For this reason, the control knobs of the adjustable embodiment include positive bidirectional detents consisting of complementary elevations and depressions which divide the rotation into discrete increments.

In the bloodstream and/or along the digestive, respiratory, or urinary tracts, real time hemodiagnostics or diagnostic apropos of the substrate ductus using implanted means can log values for numerous diagnostic indicia received from pathological condition detectors incorporated into the jackets which the implanted prescription-programed microcontroller can translate into actuation of the surface port fed subdermally positioned small flat drug reservoir outlet pumps or drip stopcocks into the direct targeting of a drug or drugs.

If the material of the catheter does not provide sufficient radiation shielding, lower dose rate radioactive drugs are magnetically carried through radiation shielded lines, as described and illustrated in U.S. Pat. No. 11,759,186, but to a nonjacketing side-entry jacket as shown in U.S. Pat. No. 11,013,858, entitled Nonjacketing Side-entry Connectors and Prosthetic Disorder Response Systems, and appear herein in FIG. 14C.

Also described in U.S. Pat. No. 11,759,186, entitled Ductus Side-entry Jackets and Prosthetic Disorder Response Systems, a permanently magnetized jacket or jackets is used to hold a paramagnetically carried drug or drugs fast against the intima, while at any level intervening between the jackets used to create the bypass, above, or below these, an impasse jacket is used. Holding he magnetized drug or drugs is with a permanently magnetized detention type impasse jacket, while draining of the magnetized drug into the luminal wall is with an electromagnetized or pull-in type impasse jacket.

If the medication used to treat the vessel superior to the bypass should be kept from the bypassed segment, the upper or entry jacket is closed off or temporarily closed off by complete retraction of its diversion chute. Keeping noxious substances from the injured segment is by periodic apheresis or dialysis (hemodialysis) as addressed below. A body surface port is then used not to complete a circuit through an extracorporeal machine but rather to provide openings into the accessory channel or channels through which to directly target medication to the bypassed segment to the upper (takeoff, proximal) of the two jackets. The jackets for such use are either conventional ductus side-entry jackets with angled trepan mainline end pieces for partial flow or adjustable diversion side-entry jackets to allow adjustment in the cross-sectional area flow between complete and partial diversion.

The accumulation of clot within the catheter used to connect the upper and lower jackets is avoided by injection or infusion of a heparin into an accessory channel of the upper jacket. The accessory channel or channels of both jackets in the same surface port, infusion of a heparin into an accessory channel of the bypass upper or takeoff jacket and its antidote (reversal agent, counteractant, neutralizer) protamine sulfate through the lower or return jacket targets and restricts the heparin to the synthetic bypass catheter so that the minimal dose is kept away from tissue. The potential for any type heparin, except, perhaps, a synthetic heparin, to induce adverse side effects are thus avoided.

The term ‘transfusion’ denotes the administration of blood or blood products taken from another and delivered by means of a hypodermic needle. ‘Reciprocal or cross transfusion’ then, denotes this interrupted manual means for exchanging blood or blood products between donor and recipient. The term ‘infusion’ pertains to drugs rather than blood or blood products administered in the same way, ruling out use of the terms ‘reciprocal or cross infusion,’ The term ‘cross-circulation’ denotes the direct and exchange of blood through a continuous circuit connecting the donor and recipient. The term ‘reciprocal cross-circulation’ then denotes that donor-recipient cross-circulation is bidirectional.

### Servovalve Toggling in Switched Organ Heterotopic Double Heart Transplantation

Heterotopic switched organ double heart transplantation is addressed below in section 2b(1)(b)(b5), entitled Heterotopic Double Heart Metered Compound Vascular Bypass, or Switched-organ Transplantation [and so on], and the Use of a Unified Entry Confluence Conduit to Avoid Disuse Atrophy when the Graft Organ is Positioned in the Lower Abdomen by Direct Aortocaval Connection. As long as reciprocal cross-circulation continues, the heart of the donor remains connected to its own circulatory system and contains a full complement of blood, about 9-½ ounces.

In an orthotopic switched-organ heart transplant at the conclusion of reciprocal cross-circulation when the native great vessels of the donor are cross-clamped and transected, the servovalves are fully opened, and the donor heart is cut off from its own circulatory system to depend upon the continuation of a normal complement of blood now supplied by the recipient.

While in an orthotopic switched-organ heart transplant this would not be expected to result in a transient reduction in donor ventricular blood volume, in a heterotopic switched-organ double heart transplant, the servovalves are left at the half-way setting with the donor heart harvested and implanted to assist the native heart of the recipient. In this circumstance, a transient reduction in graft organ blood volume would appear more plausible.

This contingency is covered by allowing control over the servovalves to alternately favor either heart disproportionately to better level an adequate volume of blood between both hearts, the process referred to as toggling. Given that throughout the process of transfer between the donor and recipient, their hearts exchange equal volumes of blood, a condition of hypovolemia in either, even early in the process, is unlikely. Nevertheless, toggling and resynchronization are mentioned out of an abundance of caution as resources available to counteract such eventualities.

This action, administered by the implanted microcontroller (or in a hierarchical arrangement to treat comorbid conditions in addition to the transplant, a microprocessor) should achieve close enough to normal volumetric flow to avoid significant donor hypovolemia as would have the potential to result in adverse consequences. Furthermore, clinical evidence has established that given sufficient time, over- and undersized transplanted hearts spontaneously adapt to a change in load, so that the need for this active apportionment of blood between the two hearts to be continued might well be ended following the initial interval during which each heart adapts to its new situation, effectively therefore, adapting as a pair where the relative size of the added heart a key factor.

Ideally, the diversion chutes can thereafter be left at the half-way point, if necessary, adjusted for size discrepancy between the hearts, or if not, then the frequency of toggling materially reduced. Should complications nevertheless arise during or after this interval despite the foregoing measures, long experience in the medical management of hypotension allows the incorporation into the prescription-program controlling the implanted automatic response system microcontroller to directly pipeline target the appropriate drugs to the hearts, implanted cardioverter-defibrillators autonomous.

Turning now to FIG. 15A which provides a schematic view of the flow pattern in reciprocal cross-circulation, the servovalves in a double heart transplant allowed to remain half open postprocedurally, the diversion of half the blood from the native heart to the added heart would suggest that for at least a brief initial interval, both hearts might remain less than fully normovolemic and provided with less than the normal volume of blood until both contain a full complement. Based upon the azygos flow principle, this might suffice for the interim until the chambers fill without necessarily triggering a ventricular fibrillation and a sudden arrest. An abundance of caution, however, dictates that a servovalve switching function of toggling be provided to alternately open the valves to either heart in quick enough succession that this eventuality would not ensue.

This ability, incorporated into the prescription program, is referred to as toggling. Toggling is also be programmed to initiate should either of the two hearts in the double transplant defibrillate. In togging, the diversion chutes are retracted and returned to the current set point frequently enough so that both organs receive essentially the normal volume of blood in alteration. This continues only until both hearts contain the normal complement of blood, about 9-½ ounces. Accordingly, toggling if it is needed at all should not be needed for more than a short time. While a one-for-one transplant of the heart allows the native vascular stumps to be anastomosed to the replacement heart, to duplicate this with the added heart in a double heart transplant is not feasible due to the native stumps having already been anastomosed.

Toggling is mentioned only as an option to cover the contingency of a brief ventricular hypovolemia that might necessitate it. On the basis of conventional heterotopic heart transplantation and the fact that hematopoiesis produces billions of new blood cells daily, this concern would appear unnecessarily cautious. Moreover, due to the additional demand for stored energy to toggle the servovalves, toggling should it prove necessary should be kept to a minimum. Venous return and ejection by toggling might be increased over that otherwise expected of either heart, especially given a vascular servovalve setting setpoint that based on the relative sizes of the hearts, would be about midway.

In unidirectional, or nonreciprocal, cross-circulation as practiced by Lillehei and his team, the heart of the volunteer, usually a parent, had to support two bodies—that of the child as a load added to that of the parent's own body. This severely limited its application. In the metered switch technique, continuously variable control over the rate of diversion chute advancement and retraction, hence, the flow rate of reciprocal cross-circulation, is essential to optimally adjust the diversion chutes for immune tolerance induction concurrent with transplantation. Such control continues as essential in double heart transplantation to apportion blood between two hearts where cardiac hypovolemia would otherwise risk a dysrhythmia if not a sudden arrest.

One approach to dispel this eventuality is by switching between one vena cava of each heart rather than both at the same time briefly and no more frequently than is necessary to keep the right atrium expelling a volume of blood, albeit less than normal. For example, a minimal volume of the terminal portion of venous return during the diastole of that heart otherwise sent 90 percent of its usual volume is switched to either the superior or inferior vena cava of the other heart. One might, for example, switch between either the superior or inferior vena cava leaving the other to flows without valving, or alternate between the superior and inferior venae cavae of either heart, laboratory testing necessary to turn such speculation into clinically applicable fact.

Since both venae cavae of both hearts are separately valved, this requires only to be entered into the control microprocessor prescription-program. It is also possible to switch between one vena cava of either heart to both of the other heart. Compared to switching or toggling almost the entirety of venous return, these schemes reduce intracardiac hypovolemia. Nevertheless, to toggle ninety percent of the venous return between the hearts at the optimal frequency determined would likely quell this problem. Based upon experience with conventional heterotopic heart transplantation, toggling is probably unnecessary. Unless excessive, a deficit of blood such as that following donation is replenished through hematopoiesis.

The adaxial surface of the foam lining is vacuum or sputter coated with a microthick surface layer of parylene too thin to interfere with the conformance of the foam to the fine vessels and nerves at the surface of the substrate vessel adventitia. Otherwise, the toxicity associated with the gradual release of breakdown products into the surrounding tissue is related to larger volumes of 2,4-toluenediamine (2,4-Diaminotoluene, TDA) administered both intravenously and subcutaneously in breast implants with a surface area considerably larger than that of the lining cushions described herein, and the testing of workers involved in the manufacture of polyurethane foam.

Nevertheless, to prevent its breakdown, the viscoelastic polyurethane foam lining is conformal surface-coated with a chemically isolating nondegradable foam-compliant barrier which consists of a biostable and biocompatible film. Such as a vapor-deposited or sputtered poly(p-xylylene) polymer material is Parylene® (Diamond-MT Corporation, Johnstown, Pennsylvania), or a similar coating film, for example.

The literature concentrates on coatings for implantable neuroelectrodes but provides relevant information as to durability and biocompatibility (see, for example, Ahn, S. H., Jeong, J., and Kim, S. J. 2019. “Emerging Encapsulation Technologies for Long-term Reliability of Microfabricated Implantable Devices,” Micromachines (Basel, Switzerland) 10(8). pii: E508; Specialty Coating Systems Corporation 2016. “Specialty Coating Systems Parylene Datasheet,” Indianapolis, Indiana: Specialty Coating Systems; Kuppusami, S. and Oskouei, R, H. 2015. “Parylene Coatings in Medical Devices and Implants: A review,” Universal Journal of Biomedical Engineering 3:9-14; Chun, W., Chou, N., Cho, S., Yang, S., and Kim, S. 2014. “Evaluation of Sub-micrometer Parylene C Films as an Insulation Layer Using Electrochemical Impedance Spectroscopy,” Progress in Organic Coatings 77:537-547; Xie, X., Rieth, L., Caldwell, R., Diwekar, M., Tathireddy, P., Sharma, R., and Solzbacher, F. 2013. “Long-term Bilayer Encapsulation Performance of Atomic Layer Deposited ALO, and Parylene C for Biomedical Implantable Devices,” Institute of Electrical and Electronics Engineers Transactions on Biomedical Engineering 60(10): 2943-2951; Noordegraaf, J. 1997. “Conformal Coating Using Parylene Polymers,” Medical Device Technology 8(1): 14-20).

Transplantation using other than the sudden compound vascular bypass, or sudden switched-organ technique requires the use of servovalves, which can be continuously adjusted to accelerate or decelerate reciprocal cross-circulation between recipient and donor or differently apportion blood between either of two hearts where only the second or both are transplants. In a sudden compound vascular bypass, or sudden switched-organ transplantation or carotid endarterectomy, the operator or an assistant depresses a small plunger switch to simultaneously actuate a number of push-pull or plunger solenoid operated valves.

In a metered orthotopic heart transplant, the switch transmits a wireless signal to the external master controller which oversees the microcontrollers or microprocessors implanted in the donor and recipient that provide the data to coordinate the reciprocal cross-circulation and concomitant release of supportive drugs in either as necessary to transfer the graft organ from the donor to the recipient. The controllers then open the diversion servovalves to initiate reciprocal cross-circulation, which the donor and recipient controllers subordinate to an extracorporeal master controller closely monitor for signs of an adverse immune response, dysrhythmia, or other disruptive sign.

In a double heart transplant, adjustment of the servovalve setpoints allows hearts different in size and ejection fraction to be paired, while toggling the valves allows the variable entrainment of a number of successive systoles of either heart to be combined thereby allowing adjustment in the net ejection fraction. It is improbable that absent toggling, that is, with the valves fixed in position at about the half-way point, the total volume of blood provided to either heart would be about half that normal, so that two hearts in one person should eventually experience a hypovolemia-induced ventricular fibrillation.

Rather the ventricular volumes should quickly normalize. Notwithstanding that the hearts are impaired in absolute stroke volume (or ‘ejection fraction’), this represents a considerable chronic underload relative to that normally driven. Preserving normal rhythm necessitates a method for alleviating the hypovolemia. One method for averting dysrhythmia, alone or in combination with other means such as pharmaceutical and mild, nonshock, electrostimulation, is diversion chute toggling to alternately deliver close to the normal volume of blood to either heart often enough to elude this consequence.

Should a dysrhythmia occur, the control microprocessor, using implantable for each heart of an, first executes an algorithm to find that action of the valves which dispels the condition most quickly. If not quickly dispelled, electrical stimulation followed by shock discharge is used. Thus, in dispelling ejection deficiency, toggling dispels an antecedent or underlying problem of chronic intracardiac hypovolemia that would induce a dysrhythmia. Toggling is programmed to preclude a dysrhythmia with the least movement of the diversion chutes, hence, the least expenditure of energy. Dispensed with is a heavy battery pack requiring frequent recharging.

When the native and donor hearts are different in size and/or ejection fraction, or absolute stroke volume, the diversion chute setpoints are adjusted accordingly, and if necessary, reciprocating motion, or toggling, of the diversion chutes to either side of their set points between partial and full extension allows differential apportionment of blood to either heart, thus causing blood to pass through both hearts frequently enough to avert the adverse consequences of intracardiac hypovolemia with eventual disuse atrophy. Toggling seeks to alternately entrain systoles from either heart into a unified bloodstream to optimize the net ejection fraction. Disuse atrophy is akin to the ischemia of a graft organ when harvested, or explanted, which leads to the injury associated with reperfusion that often results in graft failure following implantation.

In a double heart transplant, as long as the hearts beat asynchronously so that successive systoles contributed by each achieve a functional ejection fraction, which the implant sensors will report to the controller, toggling may not be needed. The probability is that left to be ‘free-running,’ the conduction system-independent hearts will drift into and out of this condition. The less often toggling support is necessary, the longer will be the intervals between the need for recharging. Otherwise, toggling may be required until the hearts and overall cardiovascular system to include its neuroendocrine control adapt to the new milieu, and if toggling is needed continuously, the power consumed is still much less than that required by mechanical assist devices.

Toggling would require immediate control over diversion chute degree of extension or open to outflow, or retraction, or closed to retention, and rates of advancement as determined on the basis of the physiological data continuously fed to the external and implanted controllers. Valves respective of specific veins and arteries in the donor and recipient, and their hearts once the donor heart is placed in the recipient, must pass the same volume of blood; however, if the hearts differ in size, the absolute degree of chute extension to send and receive the same volume may be different. Should either heart begin to malfunction, the bulk of flow can be passed through the other heart.

Using radiation shielded lines and nonjacketing side-entry connectors as shown in U.S. Pat. No. 11,013,858, entitled Nonjacketing Side-entry Connectors and Prosthetic Disorder Response Systems, for example, malignant nidi can be directly targeted with chemotherapeutics and radioactive agents, again, in higher concentration with minimal if any exposure to untargeted tissue, the means therefore involving neutralization or removal by magnetic extraction or if available, the use of a reversal agent. The direct piping of drugs to the root root, or site, of disease not only eliminates the exposure of unintended tissue but also the limitations placed upon the oral drugs, to include limited dosages and the wasteful greater cost of systemic dispersal.

Eliminating exposure of the entire body to antibiotics, for example, should significantly reduce the increased resistance of bacteria to these drugs, as well as the use of higher dosing. Inherently and unavoidably, antibiotics also kill beneficial bacteria, sometimes necessitating the use of probiotics, and immunosuppressives reduce resistance to infection. For this reason, three approaches can serve to ameliorate the adverse effects of these drugs in organ transplantation: These are:
1. Reducing the dose required as imparted by the donor-recipient reciprocal cross-microchimerization inherent in the extended donor-recipient reciprocal cross-circulation inherent in the metered compound vascular bypass technique for organ transplantation.
2. Developing drugs which are immunologically specific, and
3. Constraining the action of the drugs to circumscribed target volumes of tissue. U.S. Pat. No. 11,389,171, entitled Integrated System for the Infixion and Retrieval of Implants, delineated different methods for preventing the passage of drugs to be contained to a targeted volume of tissue from passing into the general circulation.

The benefits of a method for transplantation that reduced the need for immunosuppressants is returned to in section (b2) below, entitled Antepartum Compound vascular bypass, or sudden switched-organ Transplantation of the through which metered compound vascular bypass solid organ transplantation gradually transfers the Heart. The inherent microchimerization of extended donor-recipient reciprocal cross-circulation graft organ from the circulatory system of the donor into that of the recipient is a means for accomplishing immune tolerance induction independent of the conventional administration of such drugs. The tissue just below the level of the jacket might present an initial adverse tissue reaction that would be suppressed were an anti-inflammatory drug directly applied to the inflamed tissue.

Although some adverse reactions to glucocorticoids, such as an adverse effect on hypothalamic-pituitary-adrenal-axis function which can induce adrenal suppression and possibly Cushing's syndrome, are intrinsic and would ensue regardless of the administration route, the direct application of triamcinolone, for example, to the affected lower tract, encouraged by a direct path to the affected site, averts an injection site reaction.

Injection poses the risk of numerous adverse tissue reactions, to include the destruction of muscle and fat down to the bone at the injection site, as well as numerous other major and minor adverse events local and less local (see, for example, Salvatierra, A, R. and Alweis, R. 2016. “Permanent Hypopigmentation after Triamcinolone Injection for Tennis Elbow,” Journal of Community Hospital Internal Medicine Perspectives 6(3): 31814; Johnston, P. C., Lansang, M. C., Chatterjee, S., and Kennedy, L. 2015. “Intra-articular Glucocorticoid Injections and Their Effect on Hypothalamic-pituitary-adrenal (HPA)-Axis Function,” Endocrine 48(2): 410-416; Habib, G., Zahran, R., Najjar, R., Badarny, S., Jabbour, A., Artul, S., Hakim, G., Jabaly-Habib, H. 2015. “The Effect of Intra-articular Injection of Diprospan at the Knee Joint on the Hypothalamic-pituitary-adrenal Axis,” Swiss Medical Weekly 145: w14134; Brinks, A., Koes, B. W., Volkers, A. C., Verhaar, J. A., and Bierma-Zeinstra, S. M. 2010. “Adverse Effects of Extra-articular Corticosteroid Injections: A Systematic Review,” Online, BioMed Central Musculoskeletal Disorders 11:206; Habib, G. S. 2009. “Systemic Effects of Intra-articular Corticosteroids,” Clinical Rheumatology 28(7): 749-756; Clinical Rheumatology 28(7): 749-756; Oikarinen, A. and Autio, P. 1991. “New Aspects of the Mechanism of Corticosteroid-induced Dermal Atrophy,” Clinical and Experimental Dermatology 16(6): 416-419; Jemec, G, B. 1988. “Linear [perilymphatic] Atrophy Following Intralesional Steroid Injections,” Journal of Dermatologic Surgery and Oncology 14(1): 88-89).

The topical extension afforded by the apparatus eliminates the site concentration inherent in injection. To protect small supportive vessels and nerves at the adventitia of the ductus to be jacketed and close off any path for the luminal contents to leak out through the ostium or aperture created, the internal surface of the jacket is lined with moisture barrier-protected viscoelastic polyurethane foam. Leakage and exposure of an open wound to blood or urine can hinder healing, considerably so if colonized by a pathogen. If the ductus to be jacketed is resistant to incision, the operator can rotate, then lock, the connector in position. To protect the urothelium, the jacket shell and foam lining are perforated to provide the adventitia with open access to the surrounding milieu.

Regardless of what type ductus is treated, if needed to prevent jacket migration, the jacket shell includes suture loops to allow tacking the jacket to neighboring tissue. Notwithstanding that the foam lining is wetted with anti-inflammatory medication, usually a corticosteroid, such as cortisone, prednisone, prednisolone, dexamethasone, or triamcinolone, for example, when placed and that nonallergenic materials are used, should an adverse tissue reaction appear, counteracting medication is targeted to the site of the irritation through the accessory channel by injection at the surface port (see, for example, Kastellorizios, M., Tipnis, N., and Burgess, D. J. 2015. “Foreign Body Reaction to Subcutaneous Implants,” Advances in Experimental Medicine and Biology 865:93-108).

If the reaction is refractory to more conventional treatment, a ductus side-entry jacket with catheter leading to the surface port is laparoscopically positioned higher (superior, cephalad, craniad) on the ureter through a small incision to target palliative medication to the site of irritation. Amino-amide drugs, such as lidocaine, prilocaine, bupivacaine, iontocaine-epinephrine and other nonsteroidal anti-inflammatory drugs, such as aspirin, naproxen, 2-arylpropionic acid, and proprionic derivative drugs, such as ibuprofen, ketoprofen, fenoprofen, and flurbiprofen directly routed to the root, site, or nidus of disease or affected segment of the ductus, or if an artery, the territory supplied afford both anesthetic and anti-inflammatory function with little risk of unwanted side effects.

If necessary, an implant timing module or microcontroller can be programmed to release the medication automatically from a small flat reservoir positioned subdermally in the pectoral region. In most cases, it should not be necessary to place other jackets upstream or downstream of the ductus such as a ureter to release drugs which can be targeted into the jacket and the ductus it surrounds without without additional jackets. Accessory channel fed microneedles to release medication at the fibrous-jacket lining interface are best avoided through the use of a “soaker hose” branch from an accessory channel.

If for a certain application the use of microneedles is preferred, this can take either of two forms, the first, nanometer-sized projections mounted to a patch backing which press into the surface of the ductus and gradually release medication by diffusion rather than injection, the other replenishable, tiny hollow needles connected to an accessory channel fed drug supply line and network to each needle through which the drug or drugs are released or if of higher viscosity, injected under pressure. The need for either should be rare.

A diversion jacket to be positioned along a blood vessel to create a shunt, for example, incorporates a water jacket at the trepan line end as described in U.S. Pat. No. 11,013,858, entitled. Nonjacketing Side-entry Connectors and Prosthetic Disorder Response Systems. Once the jacket is connected, the water jacket continues in use as an accessory channel which ordinarily passes medication from an implanted drug reservoir but can be internally configured to pass through miniature cabled devices, such as atherectomizers, thrombectomizers, intravascular ultrasound probes, angioscopes, laser, and so on. Upon placement, the water jacket is used as a miniature pressure washer to restrain blood from extravasating and entering the diversion line, or mainline.

Water delivery through the water jacket is through an accessory or service channel. Once the jacket is in position, the accessory channel remains available to deliver medication into the jacket and substrate native ductus as an accessory channel. Another factor likewise due to the difference of continuous flow through a blood vessel and intermittent flow through a ureter is that the former is almost never adjustable. If adjustability is desired, it is to establish diverted flow temporarily, only so long as the native vessel needs to heal, or is meant to allow evaluation of the shunt created, or to be able to switch flow from one vessel to another is of value in research. The potential need for diversion should be taken into consideration when the system is implanted.

Nonjacketing side-entry connectors are securely anchored within the substrate tissue as to recommend additional stabilization only when the tissue is malacotic or might become so. Then anti-migration means for nonjacketing connectors as well as ductus side-entry jackets consists of tying either to the underlying structure. Suture connected to the suture loops molded into jackets and connectors about the surface is spirally wound about the ductus above and below the jacket or to a neighboring ductus with an adequate number of turns to assure stability.

When the distance between the ends of the suture is substantially constant, fixation can consist of or include suturing to neighboring tissue. In an emergency when time is lacking and connection to thicker planar rather than ductal tissue must be accomplished instantly, the use of one or more tiny barbs emulative of a honeybee stinger can be used. The use of barbs has been used to fixate implanted medical devices such as neuromodulators for decades. The ‘stinger’ is fastened at the end of suture much as a lure or fly is to fishing line (Ling, J., Song, Z., Wang, J., Chen, K., Li, J., and 5 others 2017. “Effect of Honeybee Stinger and Its Microstructured Barbs on Insertion and Pull Force,” Journal of the Mechanical Behavior of Biomedical Materials 68:173-179). The analogy can be extended to the use of a hollow barb that would allow the injection of drugs into the substrate tissue under the control of an implanted microcontroller no differently than medication is released into a side-entry jacket, valve, or connector.

When placed in a patient with frequent urination, enuresis, or nocturia, for whom access to a bathroom eliminates the need for urinary diversion, the bistable (on/off, binary) embodiment is not limited to one-time advancement unless forcibly retracted by the operator but can be advanced and retracted as necessary. One-time binary diversion valves are also suitable for use in vascular prostheses and bypasses, which unlike the urinary applications, where the patient can manually switch the valve on either side with a manually operated push/pull control cable, a number of valves must be switched once from fully retracted to fully advanced at the same instant.

Valves which must move from completely open to completely closed at the same instant, used, for example, in sudden compound vascular bypass, or sudden switched-organ transplantation and extracardiac correction of complete, or dextro-transposition, of the great arteries, incorporate highly damped nonsparking plunger solenoids controlled from a single switch. Valves controlled thus implement recipient-to-donor solid organ inflow and outflow compound vascular bypass essential to suddenly and completely switch recipient circulation from his native to the donor organ. Except in transplanting a single kidney or lung, the donor must be brain dead.

Transplantation thus is intended for use when the donor on life support is deteriorating and/or the recipient is nearing death due to end-stage organ failure. Where exigency is not a factor, metered compound vascular bypass, or sudden switched-organ transplantation uses servovalves to control the gradual movement of the diversion chutes between completely open to completely closed so that the relative proportion of blood diverted from the recipient to the graft organ is progressively increased as a means for accomplishing immune tolerance induction concurrent with transplantation.

In this way, metered switch differs from sudden compound vascular bypass, or sudden switched-organ transplantation in transferring circulation to accomplish reciprocal cross-microchimerization to reduce immunological incompatibilities from the recipient to the donor organ gradually rather than abruptly as in sudden compound vascular bypass, or sudden switched-organ transplantation. With sudden compound vascular bypass, or sudden switched-organ transplantation, blood draws and tissue samples can be manually exchanged perioperatively and intraoperatively between the donor and recipient, donors and recipient, or donor and recipients, or donors and recipients.

In FIGS. 20B and 21B, these stages at the center depict this extended transfer of reciprocal cross-circulation between recipient and donor as a stage intervening between complete separation of recipient and donor circulatory systems in FIGS. 20A and 21A and the completed transfer of perfusion from the recipient to the donor organ in FIGS. 20C and 21C. During this interval, the blood of the participants becomes more and more mixed, completely so if the process, continuously variable in speed and release of materials into the mixing circulations is continued long enough.

Bistable and controlled from the same switch, damped nonsparking plunger solenoid-controlled diversion valves slide the diversion chute from fully retracted to fully extended into the substrate native lumina to fully obturate and divert flow from these into the blood-conveying mainlines each connecting counterpart organ inflow and outflow vessels of the recipient and donor. Such valves are also used in urinary diversion as addressed below. In contrast, metered compound vascular bypass, or sudden switched-organ transplantation is accomplished with vascular servovalves, which are precisely controlled by an implanted microcontroller driven by data fed to it by implanted immune factor and other physiological sensors.

Servovalves are not limited in motional pattern or timing. The elimination of ischemia-reperfusion injury a benefit of sudden and metered compound vascular bypass, or sudden switched-organ transplantation, both plunger solenoid and servovalve operated vascular valve-jackets allow transplantation without interruption or reduction in perfusion through a single graft organ and virtually none in a metered switch double heart transplant with implanted automatic support as described in U.S. Pat. No. 11,759,186, entitled Ductus Side-entry Jackets and Prosthetic Disorder Response Systems.

In so doing, this should avert much of the damage done to the organ when the donor on life support expires and life support is shut down, at which time cytokines react in a storm or cascade to materially reduce the quality of the organ (see, for example, van Zanden, J. E.1, Jager, N. M., Daha, M. R., Erasmus, M. E., Leuvenink, H, G. D., and Seelen, M. 2019. “Complement Therapeutics in the Multi-organ Donor: Do or Don't?,” Online, Frontiers in Immunology (Lausanne, Switzerland) 10:329; Sally, M. B., Malinoski, D. J., Zaldivar, F. P., Le, T., Khoshnevis, M., and 3 others 2018. “A Porcine Model to Study the Effect of Brain Death on Kidney Genomic Responses,” Journal of Clinical and Translational Science 2(4): 208-216; Poppelaars, F. and Seelen, M, A. 2017. “Complement-mediated Inflammation and Injury in Brain Dead Organ Donors,” Molecular Immunology 84:77-83; Damman, J., Bloks, V. W., Daha, M. R., van der Most, P. J., Sanjabi, B., and 6 others 2015. “Hypoxia and Complement-and-coagulation Pathways in the Deceased Organ Donor as the Major Target for Intervention to Improve Renal Allograft Outcome,” Transplantation 99(6): 1293-1300; Dziodzio, T., Biebl, M., and Pratschke, J. 2014. “Impact of Brain Death on Ischemia/Reperfusion Injury in Liver Transplantation,” Current Opinion in Organ Transplantation 19(2): 108-114; Atkinson, C., Floerchinger, B., Qiao, F., Casey, S., Williamson, T., and 6 others 2013. “Donor Brain Death Exacerbates Complement-dependent Ischemia/Reperfusion Injury in Transplanted Hearts,” Circulation 127(12): 1290-1299; Zhang, S. J. and Wang, T. 2011. “The Influence of Brain Death on Donor Liver and the Potential Mechanisms of Protective Intervention,” Frontiers of Medicine 5(1): 8-14).

Not only administering the operation, following transplantation, the implanted prosthetic disorder response system that had been placed to treat heart failure, remains to automatically detect the need for and dispense transplant maintenance medication (see, for example, Harris, C., Cao, C., Croce, B., and Munkholm-Larsen, S. 2018. “Heart Transplantation,” Online, Annals of Cardiothoracic Surgery (Hong Kong, China) 7(1): 172; Kim, I. C., Youn, J. C., and Kobashigawa, J, A. 2018. “The Past, Present and Future of Heart Transplantation,” Korean Circulation Journal 48(7): 565-590, D'Addio, F., Margonato, D., Pensato, U., Borgese, L., Potena, L., and Fiorina, P. 2015. “Novel Therapeutic and Diagnostic Management of Heart Transplant Patients,” Heart, Lung, Vessels 7(3): 198-207. section entitled Postoperative Management after Heart Transplantation; additional references cited below under section 2b(1)(b)(b2), entitled Prenatal Compound Vascular Bypass, or Sudden Switched-organ Transplantation of the Heart).

Another value in the elimination of an interruption in perfusion past the terminal event is that the entire problem of ischemia-reperfusion injury upon placement in the donor is eliminated, and therewith, a prominent cause for both injury to other organs and tissues and graft rejection or reduced term of viability (see, for example, Zhao, H., Alam, A., Soo, A. P., George, A. J. T., and Ma, D. 2018. “Ischemia-Reperfusion Injury Reduces Long-term Renal Graft Survival: Mechanism and Beyond,” EBioMedicine [Elsevier Biomedicine] 28:31-42; Fauzia, E., Barbhuyan, T. K., Shrivastava, A. K., Kumar, M., Garg, P., and 3 others 2018. “Chick Embryo: A Preclinical Model for Understanding Ischemia-reperfusion Mechanism,” Online, Frontiers in Pharmacology 9:1034; Saat, T. C., van den Akker, E. K., IJzermans, J. N., Dor, F. J., and de Bruin, R. W. 2016. “Improving the Outcome of Kidney Transplantation by Ameliorating Renal Ischemia Reperfusion Injury: Lost in Translation?,” Journal of Translational Medicine 14:20; Kalogeris, T., Baines, C. P., Krenz, M., and Korthuis, R. J. 2016, Op cit., this section; Bellanti, F. 2016. “Ischemia-reperfusion injury: Evidences for Translational Research,” Annals of Translational Medicine 4 (Supplement 1): S55; Gonzalez, L. M., Moeser, A. J., and Blikslager, A. T. 2015. “Animal Models of Ischemia- reperfusion-induced Intestinal Injury: Progress and Promise for Translational Research,” American Journal of Physiology. Gastrointestinal and Liver Physiology 308(2): G63-G75; Nastos, C., Kalimeris, K., Papoutsidakis, N., Tasoulis, M. K., Lykoudis, P. M., and 4 others 2014. “Global Consequences of Liver Ischemia/Reperfusion Injury,” Oxidative Medicine and Cellular Longevity 2014:906965; Ponticelli, C. 2014. “Ischaemia-reperfusion Injury: A Major Protagonist in Kidney Transplantation,” Nephrology, Dialysis, Transplantation 29(6): 1134-1140; Gillani, S., Cao, J., Suzuki, T., and Hak, D. J. 2012. “The Effect of Ischemia Reperfusion Injury on Skeletal Muscle,” Injury 43(6): 670-675; Kalogeris, T., Baines, C. P., Krenz, M., and Korthuis, R. J. 2012, Op cit., this section; Khalil, A. A., Aziz, F. A., and Hall, J, C. 2006. “Reperfusion Injury,” Plastic and Reconstructive Surgery 117(3): 1024-1033).

An vascular servovalve is no less able to suddenly move the valve diversion chute from fully retracted to fully open or the reverse and therefore able to perform a sudden compound vascular bypass, or sudden switched-organ transplantation. The complexity of reperfusion injury is so multifaceted, that to reverse its consequences in detail after these consequences have already set in will likely take many more years and may never be fully achieved, making the eradication ab initio of ischemia during transplantation the far more propitious strategy. However, a vascular servovalve, driven by a small leadscrew or linear motor under the control of a microprocessor acting as the master node in a hierarchical control system, is also able to divert flow through the organ from the donor to the recipient by guarded degrees, during which the condition of both donor and recipient are tightly monitored for adverse allogeneic reactions for response by the control system.

This gradual blending of donor and recipient blood should induce or if preprocedurally carried out manually, then continue the induction of immune tolerance which the system is able to increase until either responds with an adverse reaction. In addition to tolerance induction by means of blending the bloodstreams of each, drugs and other agents can be delivered into the blood of either to mix into the blend (monoclonal antibodies, such as interleukin-2 receptor cluster of differentiation 25 and cluster of differentiation 3-targeting antibodies; cyclosporine and tacrolimus calcineurin inhibitors combined with mycophenolate mofetil; the mammalian target of rapamycin sirolimus, interferons, opioids, and so on).

The system then adjusts the valve jacket diversion chutes as directed by the prescription-program of the microcontroller or microprocessor implant, either partially or fully retracting the diversion chutes, and releases reversal and ameliorative medication such as an anti-inflammatory and/or an immunosuppressant (see, for example, Schweiger, M., Zuckermann, A., Beiras-Fernandez, A., Berchtolld-Herz, M., Boeken, U., and 10 others 2018. “A Review of Induction with Rabbit Antithymocyte Globulin in Pediatric Heart Transplant Recipients,” Annals of Transplantation 23:322-333; Saull, H. E., Enderby, C. Y., Gonwa, T. A., and Wadei, H. M. 2015. “Comparison of Alemtuzumab vs. Antithymocyte Globulin Induction Therapy in Primary Non-sensitized Renal Transplant Patients Treated with Rapid Steroid Withdrawal,” Clinical Transplantation 29(7): 573-580).

Introduction thus is by injection through a small port at the body surface leading into a valve-jacket accessory channel and therefore directly targeted to the vessel the jacket encircles. In such a control system, sensors provide physiological and/or pathophysiological data to their local nodes which process and pass the data to higher level data coordination nodes until this reaches the master node microprocessor which coordinates the data from the different reporting channels. Before transplantation is initiated, the donor is provided with such an implanted prosthetic disorder response system. The system is intended to allow the optimization of his condition, preferably avert the need for a transplant, and if so, allow the continued automatic detection and the release of maintenance drugs. Should the patient die, the system will have been prepositioned to prepare the patient for immune tolerogenic treatment to better match the prospective recipient or recipients.

Generally, in metered compound vascular bypass, or sudden switched-organ transplantation, the prosthetic disorder response systems of the deceased donor kept on life and medical support initiated before the terminal event and the recipient:
1. Monitor the baseline immune and comorbid status of both donor and recipient.
2. Regulate the delivery by dose or volume of drugs and other exogenous agents and adjust these to the optimal level for immune tolerance induction by
3. Controlling the release of drugs and
4. The rate and direction of advancement of the diversion chutes in gradually switching the graft organ from the circulatory system of the recipient to that of the donor.

To the extent possible, an organ transplant prescription-program is normalized or generalized. That is, the program is written to maintain optimal homeostasis during and following transplantation of any solid organ or combination thereof, and while adjusted for body mass and the specific organ or organs to be transplanted, is written to cover either all infant or adult individuals undergoing the same procedure. The program thus acts not as a gross adjustment preparatory routine, as would the process entrusted to medical specialists using extracorporeal apparatus and drugs, but rather as the fine adjustment mid- and postprocedural administrator of the transplantation process implemented once any indication of an index falling outside the normal range had been automatically detected and treatment initiated.

Because adults have a mature immune system and multiple ways in which the system had been challenged, the process is more complex than in infants. Then, later in life, immunity is compromised by immunosenescence, which results in greater tolerance not just for pathogens but an allogeneic solid organ. In the references cited just below, the 2016 paper by Chih, and Patel is of special importance for adult transplantation.

Provided maternal or paternal chimerization are medically insignificant, the immune system in a neonate is premature so that immune tolerance induction through metered rather than sudden compound vascular bypass, or sudden switched-organ transplantation should be avoidable, and associated defects notwithstanding, comorbidity as such is seldom a complication, whereas in an adult, concurrent, or comorbid, disease with inflammation and often infection pose a preliminary problem of existing antibody sensitization (see, for example, Colvin, M. M., Cook, J, L., Chang, P. P., Hsu, D. T., Kieman, M. S., and 7 others 2019. “Sensitization in Heart Transplantation: Emerging Knowledge: A Scientific Statement from the American Heart Association,” Circulation 139(12): e553-e578; Alsughayyir, J., Chhabra, M., Qureshi, M. S., Mallik, M., Ali, J. M., and 8 others 2019. “Relative Frequencies of Alloantigen-specific Helper CD4 T Cells and B Cells Determine Mode of Antibody-mediated Allograft Rejection,” Online, Frontiers in Immunology (Lausanne, Switzerland) 9:3039; Chhabra, M., Alsughayyir, J., Qureshi, M. S., Mallik, M., Ali, J. M., and 8 others 2019. “Germinal Center Alloantibody Responses Mediate Progression of Chronic Allograft Injury,” Online, Frontiers in Immunology (Lausanne, Switzerland) 9:3038; Li, W., Gauthier, J. M., Higashikubo, R., Hsiao, H. M., Tanaka, S., and 14 others 2019. “Bronchus-associated Lymphoid Tissue-resident Foxp3+ T Lymphocytes Prevent Antibody-mediated Lung Rejection,” Journal of Clinical Investigation 129(2): 556-568; van Zanden, J, E. 1, Jager, N. M., Daha, M. R., Erasmus, M. E., Leuvenink, H, G. D., and Seelen, M. 2019. Op cit., this section; Kolonko, A., Bzoma, B., Giza. P., Styrc. B., Sobolewski, M., and 3 others 2018. “The Pre-transplant Drop in Panel-reactive Antibodies Titer Evaluated Using Complement-dependent Cytotoxicity (PRA-CDC) and the Risk of Early Acute Rejection in Sensitized Kidney Transplant Recipients,” Medicina (Kaunas, Lithuania) 2018 54(5). pii: E66; Hachem, R. R., Kamoun, M., Budev, M. M., Askar, M., Ahya, V. N., and 12 others 2018. “Human Leukocyte Antigens Antibodies after Lung Transplantation: Primary Results of the HALT [radiotherapy for oligo-progressive disease in oncogene-Addicted Lung Tumours] Study,” American Journal of Transplantation 18(9): 2285-2294; Siu, J, H. Y., Park, J. E., Kim, C. Y., Park, M. S., Song, J. H., Kim, Y. S., and 3 others 2018. “Prevalence of Pre-transplant Anti-HLA Antibodies and Their Impact on Outcomes in Lung Transplant Recipients,” Online, BioMed Central Pulmonary Medicine 18(1): 45; Surendrakumar, V., Richards, J. A., and Pettigrew, G. J. 2018. T Cell Allorecognition Pathways in Solid Organ Transplantation,” Online, Frontiers in Immunology (Lausanne, Switzerland) 9:2548; Okada, D., Okumi, M., Kakuta, Y., Unagami, K., Iizuka, J., and 3 others 2018. “Outcome of the Risk-stratified Desensitization Protocol in Donor-specific Antibody-positive Living Kidney Transplant Recipients: A Retrospective Study,” Transplant International April 20; Nuñez, K., Thevenot, P., Alfadhli. A., and Cohen, A. 2018. “Complement Activation in Liver Transplantation: Role of Donor Macrosteatosis and Implications in Delayed Graft Function,” Online, MDPI [Multidisciplinary Digital Publishing Institute] International Journal of Molecular Sciences (Basel, Switzerland) 19(6). pii: E1750; Liang, E. C., Rossetti, M., Sidwell. T., Groysberg. V., Sunga, G., and 9 others 2018. “Differences in Proinflammatory Cytokines and Monocyte Subtypes in Older as Compared with Younger Kidney Transplant Recipients,” Transplantation Direct 4(3): e348; Nishimura, H., Yamada, Y., Hisano, S., Mitsuke, A., Tatarano, S., and 4 others 2018. “Long-term Desensitization for ABO-incompatible Living Related Kidney Transplantation Recipients with High Refractory and Rebound Anti-blood Type Antibody: Case Report,” Online, BioMed Central Nephrology 19(1): 254; Kim, Y. C., Yu, M. Y., Lee, J. P., Lee, H., Min, S. I., Ha, J., and Kim, Y. S. 2018. “The Effect of Desensitization Therapy in Kidney Transplantation,” Clinical and Experimental Nephrology 22(1): 179-187; Lee, D. R., Kim, B. C., Kim, J. P., Kim, I. G., and Jeon, M. Y. 2018. “C3d-binding Donor-specific HLA Antibody is Associated with a High Risk of Antibody-mediated Rejection and Graft Loss in Stable Kidney Transplant Recipients: A Single-center Cohort Study,” Transplantation Proceedings 50(10): 3452-3459; Schaenman, J. M., Rossetti, M., Sidwell, T., Groysberg. V., Sunga, G., and 9 others 2018. “Increased T Cell Immunosenescence and Accelerated Maturation Phenotypes in Older Kidney Transplant Recipients,” Human Immunology 79(9): 659-667; Bansal, S., Sharma, M., R. R., and Mohanakumar, T. 2018. “The Role of Exosomes in Allograft Immunity,” Cellular Immunology 331:85-92; Gunasekaran, M., Sharma, M., Hachem, R., Bremner, R., Smith, M. A., and Mohanakumar. T. 2018. “Circulating Exosomes with Distinct Properties during Chronic Lung Allograft Rejection,” Journal of Immunology (Baltimore, Maryland) 200(8): 2535-2541; Nakamura, Y., Yoshioka, D., Miyagawa, S., Yoshikawa, Y., Hata, H., and 3 others 2018. “Successful Heart Transplantation after Desensitization in a Patient with Extremely High Panel-reactive Antibody Levels and Pretransplant Donor-specific Antibody: A Case Report,” Transplantation Proceedings 50(10): 4067-4070; Roux, A., Thomas, K. A., Sage, E., Suberbielle-Boissel, C., Beaumont-Azuar, L., and 5 others 2018. “Donor-specific HLA Antibody-mediated Complement Activation is a Significant Indicator of Antibody-mediated Rejection and Poor Long-term Graft Outcome during Lung Transplantation: A Single Center Cohort Study,” Transplant International 31(7): 761-772; Ixtlapale-Carmona, X., Arvizu, A., De-Santiago, A., Gonzalez-Tableros, N., Lopez, M., and 7 others 2018. “Graft Immunologic Events in Deceased Donor Kidney Transplant Recipients with Preformed HLA-donor Specific Antibodies,” Transplantation Immunology 46:8-13; Aguilar, P. R., Carpenter. D., Ritter, J., Yusen, R. D., Witt, C. A., and 5 others 2018. “The Role of C4d [complement component 4] Deposition in the Diagnosis of Antibody-mediated Rejection after Lung Transplantation,” American Journal of Transplantation 18(4): 936-944; Dipchand, A. I., Webber, S., Mason, K., Feingold, B., Bentlejewski, C., and 12 others 2018. “Incidence, Characterization, and Impact of Newly Detected Donor-specific Anti-HLA Antibody in the First Year after Pediatric Heart Transplantation: A Report from the CTOTC-04 [Clinical Trials in Organ Transplantation in Children-04] Study,” American Journal of Transplantation 18(9): 2163-2174; Health Jade 2018. ““Plasmapheresis,” Online at https://healthjade.com/plasmapheresis/; Chen, C. C., Pouliquen, E., Broisat, A., Andreata, F., Racape, M., and 22 others 2018. “Endothelial Chimerism and Vascular Sequestration Protect Pancreatic Islet Grafts from Antibody-mediated Rejection,” Journal of Clinical Investigation 128(1): 219-232; Dangi, A., Yu. S., and Luo, X. 2018. “Apoptotic Cell-based Therapies for Promoting Transplantation Tolerance,” Current Opinion in Organ Transplantation 23(5): 552-558; Malvezzi, P., Jouve, T., Noble, J., and Rostaing. L. 2018. “Desensitization in the Setting of HLA-incompatible Kidney Transplant,” Experimental and Clinical Transplantation 16(4): 367-375; Bailly, E., Anglicheau, D., Blancho, G., Gatault, P., Vuiblet. V., and 18 others 2018. “Prognostic Value of the Persistence of C1q-binding Anti-HLA Antibodies in Acute Antibody-mediated Rejection in Kidney Transplantation,” Transplantation 102(4): 688-698; Zhang, Q., Hickey, M., Drogalis-Kim, D., Zheng. Y., Gjertson, D., and 15 others 2018. “Understanding the Correlation between DSA [donor-specific HLA antibodies], Complement Activation, and Antibody-mediated Rejection in Heart Transplant Recipients,” Transplantation 102(10): e431-e438; Yang, Y., Liu, B., Xu. J., Wang. J., Wu. J., and 30 others 2017. “Derivation of Pluripotent Stem Cells with In Vivo Embryonic and Extraembryonic Potency,” Cell 169(2): 243-257.e25; Lee, K. W., Park. J. B., Cho, C. W., Lee. N., Yoo. H., and 5 others 2017. “The Impact of Donor-specific Anti-human Leukocyte Antigen (HLA) Antibody Rebound on the Risk of Antibody Mediated Rejection in Sensitized Kidney Transplant Recipients,” Annals of Transplantation 22:166-176; Chang, D, H. and Kobashigawa, J, A. 2017. “Desensitization Strategies in the Patient Awaiting Heart Transplantation,” Current Opinion in Cardiology February 15; Geft, D. and Kobashigawa, J. 2017. “Current Concepts for Sensitized Patients before Transplantation,” Current Opinion in Organ Transplantation 22(3): 236-241; Poppelaars, F. and Seelen, M, A. 2017. “Complement-mediated Inflammation and Injury in Brain dead Organ Donors,” Molecular Immunology 84:77-83; Gilbert. O. N. and Chang, P. P. 2017. “The Approach to Antibodies after Heart Transplantation,” Current Transplantation Reports 4(3): 243-251; Krenzien, F., Quante, M., Heinbokel, T., Seyda, M., Minami, K., and 12 others 2017. “Age-dependent Metabolic and Immunosuppressant Effects of Tacrolimus,” American Journal of Transplantation 17(5): 1242-1254; van Gelder, T. 2017. “Biomarkers in Solid Organ Transplantation,” British Journal of Clinical Pharmacology 83(12): 2602-2604; Cheng, E. Y. 2017. “The Role of Humoral Alloreactivity in Liver Transplantation: Lessons Learned and New Perspectives,” Journal of Immunology Research 2017:3234906; Sethi, S., Choi, J., Toyoda, M., Vo. A., Peng. A., and Jordan, S, C. 2017. “Desensitization: Overcoming the Immunologic Barriers to Transplantation,” Journal of Immunology Research 2017:6804678; Colvin, M. M., Smith, C. A., Tullius, S. G., and Goldstein, D, R. 2017. “Aging and the Immune Response to Organ Transplantation,” Journal of Clinical Investigation 127(7): 2523-2529; Irure, J., Asensio, E., Rodrigo, E., Romon, I., Gomez, J., and 3 others 2017. “Improvement in the Definition of Anti-HLA Antibody Profile in Highly Sensitized Patients,” Online, PLoS [Public Library of Science] One 12(2): e0171463; Cheng, E. Y., Everly, M. J., Kaneku, H., Banuelos, N., Wozniak, L. J., and 6 others 2017. “Prevalence and Clinical Impact of Donor-specific Alloantibody among Intestinal Transplant Recipients,” Transplantation 2017 101(4): 873-882; Tyan, D, B. 2017. “Application, Technical Issues, and Interpretation of C1q for Graft Outcome,” Current Opinion in Organ Transplantation 22(5): 505-510; Malheiro, J., Tafulo. S., Dias, L., Martins, S., Fonseca, I., and 3 others 2017. “Determining Donor-specific Antibody C1q-binding Ability Improves the Prediction of Antibody-mediated Rejection in Human Leucocyte Antigen-incompatible Kidney Transplantation,” Transplant International 30(4): 347-359; Mangiola, M., Marrari, M., Feingold, B., and Zeevi, A. 2017. “Significance of Anti-HLA Antibodies on Adult and Pediatric Heart Allograft Outcomes,” Online, Frontiers in Immunology (Lausanne, Switzerland) 8:4; Valenzuela, N. M. and Reed, E, F. 2017. “Antibody-mediated Rejection across Solid Organ Transplants: Manifestations, Mechanisms, and Therapies,” Journal of Clinical Investigation 127(7): 2492-2504; Sethi, S., Choi, J., Toyoda, M., Vo. A., Peng. A., and Jordan, S, C. 2017. “Desensitization: Overcoming the Immunologic Barriers to Transplantation,” Journal of Immunology Research 2017:6804678; Ravindranath, M, H. 2017. “HLA Class Ia and Ib Polyreactive Anti-HLA-E IgG2a Monoclonal Antibodies (TFL-006 and TFL-007) Suppress Anti-HLA IgG Production by CD19+ B Cells and Proliferation of CD4+ T Cells While Upregulating Tregs,” Journal of Immunology Research 2017:3475926; Wozniak, L. J., Naini, B. V., Hickey, M. J., Bhattacharyya, S., Reed, E. F., and 5 others 2017. Op cit. under section 2a above entitled Background of the Invention. General; Chih. S. and Patel, J. 2016. “Desensitization Strategies in Adult Heart Transplantation-Will Persistence Pay Off?,” Journal of Heart and Lung Transplantation (New York, New York) 35(8): 962-972; Malheiro, J., Martins, L. S., Tafulo. S., Dias, L., Fonseca, I., and 3 others 2016. “Impact of De Novo Donor-specific Anti-HLA Antibodies on Grafts Outcomes in Simultaneous Pancreas-Kidney Transplantation,” Transplant International 29(2): 173-183; Svobodova, E., Gazdic. T., Kubanek, M., Vymetalova, J., Voska, L., and 10 others 2016. “Novel Insights into Pretransplant Allosensitization in Heart Transplant Recipients in the Contemporary Era of Immunosuppression and Rejection Surveillance,” Transplant International 29(1): 63-72; Sharma. A., King. A., Kumar, D., Behnke, M., McDougan, F., and Kimball. P. M. 2016. “Perioperative Desensitization Improves Outcomes among Crossmatch Positive Recipients of Deceased Donor Renal Transplants,” Progress in Transplantation 26(2): 157-161; Ding. S., Xie, J., and Wan, Q. 2016. “Association between Cytokines and their Receptor Antagonist Gene Polymorphisms and Clinical Risk Factors and Acute Rejection Following Renal Transplantation,” Medical Science Monitor 22:4736-4741; Valenzuela, N. M., Hickey, M. J., and Reed, E, F. 2016. “Antibody Subclass Repertoire and Graft Outcome following Solid Organ Transplantation,” Online, Frontiers in Immunology (Lausanne, Switzerland) 7:433; Keverne, E, B. 2015. “Genomic Imprinting, Action, and Interaction of Maternal and Fetal Genomes,” Online, Proceedings of the National Academy of Sciences of the United States of America 112(22): 6834-6840; Cuadrado. A., San Segundo, D., Lopez-Hoyos, M., Crespo, J., and Fábrega, E. 2015. “Clinical Significance of Donor-specific Human Leukocyte Antigen Antibodies in Liver Transplantation,” World Journal of Gastroenterology 21(39): 11016-11026; Molnar, A. O., Fergusson, D., Tsampalieros, A. K., Bennett, A., Fergusson, N., Ramsay, T., and Knoll, G, A. 2015. “Generic Immunosuppression in Solid Organ Transplantation: Systematic Review and Meta-analysis,” Online, British Medical Journal (Clinical Research) 350: h3163; Klinger, M. and Banasik, M. 2015. “Immunological Characteristics of the Elderly Allograft Recipient,” Transplantation Reviews (Orlando, Florida) 29(4): 219-223; Zachary, A, A. and Leffell, M. S. 2014. “Desensitization for Solid Organ and Hematopoietic Stem Cell Transplantation,” Immunological Reviews 258(1): 183-207; Lobashevsky, A. L. 2014. “Methodological Aspects of Anti-human Leukocyte Antigen Antibody Analysis in Solid Organ Transplantation,” Online, World Journal of Transplantation (Pleasanton, California) 4(3): 153-167; Zheng, J., Xue, W., Qing, X., Jing, X., Hou, J., and 4 others 2014. “Treatment of Biopsy-proven Acute Antibody-mediated Rejection Using Thymoglobulin [antithymocyte globulin] (ATG) Monotherapy and a Combination of Rituximab, Intravenous Immunoglobulin, and Plasmapheresis: Lesson Learned from Primary Experience,” Clinical Transplants 2014:223-230; Zhu, D., Qi. G., Tang, Q., Li, L., Yang. C., and 6 others 2014. “Combination of Total Lymphoid Irradiation, Low-dose IVIG [intravenous immunoglobulin] and ATG [antithymocyte globulin] as Rescue Therapy for Highly Sensitized and Antibody-mediated Rejection Renal Transplant Recipients,” Clinical Transplants 2014:215-221; Kimball, P. M., McDougan, F. A., and King, A. 2014. “Effects of Two Preemptive Post-transplant Desensitization Regimens upon Renal Allograft Survival and DSA [donor specific antibodies] Elaboration,” Clinical Transplants 2014:197-203; Cheng, E. Y., Kaneku, H., and Farmer, D, G. 2014. “The Role of Donor-specific Antibodies in Intestinal Transplantation: Experience at the University of California Los Angeles and Literature Review,” Clinical Transplants 2014:153-159; Jackson, A. M. 2014. “The Virtual Crossmatch: An Essential Tool for Transplanting Sensitized Patients,” Clinical Transplants 2014:131-136; Pankewycz, O., Soliman, K., and Laftavi, M, R. 2014. “The Increasing Clinical Importance of Alloantibodies in Kidney Transplantation,” Immunological Investigations 43(8): 775-789; Everly, M. J. 2014. “Update on Alloantibodies in Solid Organ Transplantation,” Clinical Transplantation 125-129; Iyer, H. S., Jackson, A. M., Zachary, A. A., and Montgomery, R, A. 2013. “Transplanting the Highly Sensitized Patient: Trials and Tribulations,” Current Opinion in Nephrology and Hypertension 22(6): 681-688; Heinbokel, T., Elkhal, A., Liu G., Edtinger, K., and Tullius, S, G. 2013. “Immunosenescence and Organ Transplantation,” Transplantation Reviews (Orlando, Florida) 27(3): 65-75; Cantone, I. and Fisher, A, G. 2013. “Epigenetic Programming and Reprogramming during Development,” Nature Structural and Molecular Biology 20(3): 282-289; Yabu, J. M., Pando, M. J., Busque, S., and Melcher, M. L. 2013. “Desensitization Combined with Paired Exchange Leads to Successful Transplantation in Highly Sensitized Kidney Transplant Recipients: Strategy and Report of Five Cases,” Transplantation Proceedings 45(1): 82-87; Knoll, G, A. 2013. “Kidney Transplantation in the Older Adult,” American Journal of Kidney Diseases 61(5): 790-797; Jin, M. K., Cho, J. H., Kwon, O., Hong, K. D., Choi, J. Y., and 4 others 2012. “Successful Kidney Transplantation after Desensitization Using Plasmapheresis, Low-dose Intravenous Immunoglobulin, and Rituximab in Highly Sensitized Patients: A Single-center Experience,” Transplantation Proceedings 44(1): 200-203; Gavranić, B. B., Basic-Jukic, N., Ratković-Gusić, I., Martinez, N., Ivancan, V., and Kes, P. 2011. “Preparation of Highly HLA-sensitized Patient for Heart Transplantation: First Documented Case in Croatia,” (in Croatian with English abstract at Pubmed) Acta Medica Croatica 65(4): 301-304; Montgomery, R. A., Lonze, B. E., and Jackson, A. M. 2011. “Using Donor Exchange Paradigms with Desensitization to Enhance Transplant Rates among highly Sensitized Patients,” Current Opinion in Organ Transplantation 16(4): 439-443; West. L. J. 2011. “ABO-incompatible Hearts for Infant Transplantation,” Current Opinion in Organ Transplantation 16(5): 548-554; Patel, J., Everly, M., Chang, D., Kittleson, M., Reed. E., and Kobashigawa, J. 2011. “Reduction of Alloantibodies via Proteasome Inhibition in Cardiac Transplantation,” Journal of Heart and Lung Transplantation (New York, New York) 30(12): 1320-1326; Asante-Korang, A., Jacobs, J. P., Ringewald, J., Carapellucci, J., Rosenberg, K., and 6 others 2011. “Management of Children Undergoing Cardiac Transplantation with High Panel Reactive Antibodies,” Cardiology in the Young (Cambridge, England) 21 Supplement 2:124-132; Bohmig, G. A., Wahrmann, M., and Bartel, G. 2011. “Transplantation of the Broadly Sensitized Patient: What are the Options?,” Current Opinion in Organ Transplantation 16(6): 588-593; Yuan, X. P., Wang, C. X., Gao, W., Fu, Q., and He, X. S. 2010. “Kidney Transplant in Highly Sensitized Patients after Desensitization with Plasmapheresis and Low-dose Intravenous Immunoglobulin,” Experimental and Clinical Transplantation 8(2): 130-135; Trzonkowski, P., Debska-Slizien, A., Jankowska, M., Wardowska, A., Carvalho-Gaspar, M., and 7 others 2010. “Immunosenescence Increases the Rate of Acceptance of Kidney Allotransplants in Elderly Recipients through Exhaustion of CD4+ T-cells,” Mechanisms of Ageing and Development 131(2): 96-104).

While the implanted process control units of both the deceased donor and recipient could be made to command extracorporeal machines to apply processes such as liver dialysis, plasmapheresis, leukapheresis, and kidney dialysis, or release volumes of agents too large for an implanted reservoir, this is not accomplished either mid- or post-procedurally using implanted means. Further, of necessity, since far too many sensors would be required to monitor and respond to cytokines in detail, for example, many if not most of which would be susceptible to the same treatment in any event, the program, in complete compliance with accepted practice, is written to the empirical or summary level to detect and respond to inflammation with anti-inflammatory medication targeted directly to the treatment site or sites.

More often, intercurrent or concomitant requirements of individual patients are entrusted to the more broad and detailed expertise of medical staff physicians, to include a hematologist, endocrinologist, internist, and immunologist. Infant and adult patients requiring a heart transplant differ not only in the absolute doses of medication required and the common causes therefor such as a heart severely malformed in an infant and end-stage heart failure in adults but also in the degree of preparation essential to proceed with the transplant. Distinctions in body mass and the specific procedure embarked upon, the control programs for both are essentially the same.

Nevertheless, where drug volumes to treat concomitant disease are small, programs for adults can differ from those written for infants in incorporating these drugs into the program as a callable unit (subroutine, subprogram). Empirically again, agents used to eradicate pathogens in adults presenting intercurrent disease might include an antimicrobial or antifungal, and those affected with concomitant disease might include antibody desensitizing agents, such as tacrolimus, mycophenolate mofetil, or rituximab, intravenous immune globulin and preoperative apheresis and/or dialysis relegated to extracorporeal equipment.

When recipient and since expired donor are connected for transplantation and possibly cross-tolerance induction, the master node microprocessor of both is placed as subordinate to the control of an extracorporeal monitor/processor. The prospective recipient having lived with a failing heart over a period such that organ impairment secondary thereto, such as cardiorenal syndrome and/or congestive hepatopathy are likely, once the transplantation procedure has been completed, the microprocessor of the recipient once again becomes the master node, executing a prescription-program devised to maintain the graft organ and treat any additional morbidities with the automatic release of drugs as necessary and assure optimal overall homeostasis for the host.

To simplify the induction of immune tolerance, where secondary organ impairment is severe, the impaired organs are considered for transplantation from the same donor. The implant system in the brain-dead donor is recovered after all organs slated for harvesting have been transplanted. To continue targeted diagnostics and therapeutics of the organ once transplanted, diversion jackets and sensors are transplanted with the substrate organ. Both in the donor and the recipient before and after transplantation, autonomous microcontroller circuits can be placed under the higher control by a microprocessor in a hierarchical control system to optimize overall homeostasis by applying treatment across the different sources of morbidity in both donor and recipient.

By switching the blood supply and drainage of the recipient from his own diseased organ to that of the donor, sudden and metered compound vascular bypass, or sudden switched-organ transplantation eliminate any interruption in perfusion, cross-clamping, and the need for cardiopulmonary support for the recipient, which involves circumventing the heart of the recipient, much less cold storage of the graft organ in a chilled static or flowing perfusate. Transplantation using these techniques therefore avoids reperfusion injury, which along with the risk of rejection, are the main sources for most of the complications encountered in solid organ transplantation.

Valves that must be precisely controllable in a graduated manner to adjust blood pressure through a particular vessel such as the main pulmonary artery or pulmonary trunk or if separately valved, its branches, for example, are controlled by a miniature linear motor or leadscrew. Any ductus side-entry jacket or nonjacketing side-entry connector can be used to target chimerizing and antirejection medication to a transplanted no less than to target such medication or antimicrobial or anti-inflammatory medication directly to a native organ or release this or any other kind of medication into the systemic and pulmonary circulation.

Sustainment of the donor affords an interval to identify a potential organ recipient or recipients and initiate chimerization and supportive therapy of the prospective donor organ or organs as well as to optimize his condition. Because the chimerizing material is directly pipeline targeted to each organ with relatively little spillover into the systemic circulation, the ability to differentially chimerize each organ for different recipients is increased. Direct targeting is of considerable benefit with any drug, but especially so with immunosuppressants, anticancer drugs, and antibiotics that administered orally would destroy beneficial microbiota in the gut.

This is so of the case depicted in FIG. 107 of U.S. Pat. No. 11,389,171, entitled Integrated System for the Infixion and Retrieval of Implants, wherewith a bacterial infection within the urinary bladder can be directly targeted for instillation of an antibiotic through a nonjacketing side-entry connector as described in more detail in U.S. Pat. No. 11,013,858, which as explained and illustrated in U.S. Pat. No. 11,759,186 and copending application Ser. No. 18/358,252, entitled Fully Implanted Aphereter/Dialyzers for Intracorporeal Blood Purification, is then released directly into the bladder for voiding without having been passed through the digestive tract. In sudden and metered compound vascular bypass, or sudden switched-organ transplantation of the heart, provided the body mass of the donor is not significantly less than that of the recipient, the heart of neither is ever confronted with an increase in load; rather, each heart supports only one body at a time.

For this reason, only wide differences in body mass between donor and recipient necessitate the use of pumps to achieve a suitable flow rate. Moreover, when switched into the circulatory system of the recipient, the much more highly competent new heart is all the more competent to its new body. Furthermore, Lillehei's first operation required that the additional load of the child be sustained for 19 minutes. His later operations were to repair significantly more serious malformities than a large septal defect, and the operations took longer than 19 minutes.

In comparison, the interval over which the heart of the recipient must send the full complement of blood to the heart of the donor is that following the switching of flow from his own defective heart to and through the donor heart plus the time to place and anastomose the donor heart in the chest of recipient. Since initial anastomosis can be interim accomplished with a surgical grade cyanoacrylate cement without waiting until the anastomoses have been sutured, the overall interval over which the additional length of tubing must be cleared runs from switching of the vascular valves until the new organ is secured in the recipient, an interval lasting substantially less than 19 minutes. While unlikely, a need to interpose a small pump along the connecting lines would be obvious to an experienced operator and quickly satisfied.

In open heart surgery, machine support is essential because the heart is disabled. In sudden compound vascular bypass, or sudden switched-organ transplantation, the organ is never disabled; rather it is just switched from the recipient to the donor. In any transplant operation where the mainlines connecting the valve-jackets must be too long to implant with the graft organ and left in place indefinitely as prosthetic, direct end-to-end connect jackets are used. Such jackets have raised rims about the ends of the jackets facing one another which are locked in end-to-end edge apposition by means of a miniaturized version of a factory drum lid type rim/cover surround lock clamp. Before connecting the ends, circulation continues to bypass the junction through the diversion chutes.

The stumps of the vessels to be anastomosed are trimmed back to about two millimeters in extension past the facing jacket ends and the raised rims at the jacket facing ends clamped together with the slightly overextended edges of the vessel positioned flush together, coated with a substance such as sterile Tualang honey [a multifloral jungle honey produced by the bee Apis dorsata in nests high in the branches of the tualang tree], found to facilitate the healing of anastomoses (Aznan, M. I., Khan, O. H., Unar, A. O., Tuan Sharif, S. E., Khan, A. H., Syed Abd Aziz, S. H., and Zakaria, A, D. 2016. “Effect of Tualang Honey on the Anastomotic Wound Healing in Large Bowel Anastomosis in Rats-A Randomized Controlled Trial,” Online, BioMed Central Complementary and Alternative Medicine (London, England) 16:28), and bonded with a surgical cyanoacrylate or fibrin cement. The balance of the anastomosing is devised to allow healing without the need for reentry. The edges of the vessels thus fused together to form a continuous conduit across the jacket junction, the mainlines are removed and the junction allowed to heal together within the time the cement will continue to bond the juxtaposed edges.

In most instances, it is preferable not to remove the valve-jackets, because the accessory channels allow the direct pipeline targeting of stem cells, an anesthetic, thus avoiding nonsteroidal anti-inflammatory drugs which weaken anastomoses, anticoagulant, and/or antimicrobial as necessary by injection into the small port at the body surface during healing and at any time thereafter (see, for example, Moussa, L., Demarquay, C., Rethore, G., Benadjaoud, M. A., Sineriz, F., and 5 others 2019. “Heparan Sulfate Mimetics: A New Way to Optimize Therapeutic Effects of Hydrogel-embedded Mesenchymal Stromal Cells in Colonic Radiation-induced Damage,” (Nature Research) Scientific Reports 9(1): 164; Kandaswamy, E. and Zuo. L. 2018. “Recent Advances in Treatment of Coronary Artery Disease: Role of Science and Technology,” Online, MDPI [Multidisciplinary Digital Publishing Institute] International Journal of Molecular Sciences (Basel, Switzerland) 19(2). pii: E424; Wada. T., Kawada, K., Hirai, K., Toda, K., Iwamoto. M., Hasegawa, S., and Sakai, Y. 2018. “Enhanced Anastomotic Healing by Daikenchuto [traditional Japanese herbal medicine] (TJ-100) in Rats,” Nature Research. Scientific Reports 8(1): 1091; Raptis, D., Pramateftakis, M. G., and Kanellos, I. 2018. “Our 20-year Experience with Experimental Colonic Anastomotic Healing,” Journal of Medicine and Life 11(1): 5-14; Stergios, K., Kontzoglou, K., Pergialiotis, V., Korou, L. M., Frountzas, M., and 3 others 2017. “The Potential Effect of Biological Sealants on Colorectal Anastomosis Healing in Experimental Research Involving Severe Diabetes,” Annals of the Royal College of Surgeons of England 99(3): 189-192; Albayrak, G., Silistreli, E., Ergur. B., Kalkan, S., Karabay, O., Erdal, A. C., and Acikel, U. 2015. “Inhibitory Effect of Adenosine on Intimal Hyperplasia and Proliferation of Smooth Muscle Cells in a Carotid Arterial Anastomosis Animal Model,” Vascular 23(2): 124-131; Nordentoft, T. 2015. “Sealing of Gastrointestinal Anastomoses with Fibrin Glue Coated Collagen Patch,” Danish Medical Journal 62(5). pii: B5081; Oines, M. N., Krarup, P. M., Jorgensen, L. N., and Agren, M. S. 2014. “Pharmacological Interventions for Improved Colonic Anastomotic Healing: A Meta-analysis,” World Journal of Gastroenterology 20(35): 12637-12648; Guzeloglu, M., Aykut, K., Albayrak, G., Atmaca, S., Oktar, S., Bagriyanik, A., and Hazan, E. 2013. “Effect of Tadalafil on Neointimal Hyperplasia in a Rabbit Carotid Artery Anastomosis Model,” Annals of Thoracic and Cardiovascular Surgery 19(6): 468-474; Sevim, K. Z., Silistreli, O., Gorgu, M., Sevim, O., and Ergur, B. 2012. “Short-term Vasculoprotective Effects of Imatinib Mesylate on Intimal Hyperplasia of Arterial Anastomosis: An Experimental Study Using a Rabbit Model,” Canadian Journal of Plastic Surgery 20(4): 223-228).

Such means are most pertinent to solid organ transplantation in prenates, neonates, and infants, where time for such measures as tissue expansion is lacking and space is a major consideration. Thus, reciprocal cross-circulation should not be misconstrued as the unilateral support of a patient by a larger volunteer whose heart is used to support the patient as well as himself with the aid of a pump positioned along the line connecting the two. The term ‘reciprocal cross-circulation’ is used to distinguish the unilateral form of cross-circulation used by C. Walton Lillehei to allow open heart surgery from the bilateral form meant here. Thus, where the term ‘cross-circulation’ denotes unilateral supply with substantially passive return, ‘reciprocal cross-circulation’ as used here denotes two-way active exchange.

With the advent of ventilators and improved cardiopulmonary bypass machines, historical cross-circulation has been abandoned, largely because it exposes the voluntary host, or volunteer to too great a risk and is limited to a volunteer much larger than the patient. It survives in other forms where the risks for both volunteer, if applicable, and patient are less severe (see, for example, O'Neill. J. D., Guenthart, B. A., Kim, J., Chicotka, S., Queen, D., and 8 others 2017. “Cross-circulation for Extracorporeal Support and Recovery of the Lung,” Nature Biomedical Engineering, Article 0037; Kitamura, M., Shibuya, M., Kurihara, H., Akimoto, T. Endo, M., and Koyanagi, H. 1997. “Effective Cross-circulation Technique of Venoarterial Bypass for Differential Hypoxia Condition,” Artificial Organs 21(7): 786-88). Here, the donor is beyond injury and the heart of the recipient suddenly replaced with that of the donor.

A side-entry diversion jacket can be used not only to redirect flow, but can incorporate components that provide imaging capability, diagnostic data, and by delivering a drug or drugs at and distal to the site where it is positioned, therapy. Moreover, while not incorporated as an integral component of the jacket, cabled devices such as an excimer laser, fine fiberscope, intravascular ultrasound probe and so on can be passed through an accessory channel or channels of the jacket to provide increased viewing and treatment capability. Ingestible magnetized drugs intended to enter the bloodstream and/or urine are in development.

Ferrofluidic drug application or the inducement into the luminal wall by a conventional or a flow-diverting side-entry jacket adapted to function additionally as an impasse jacket applies to any bodily conduit, whether urinary, reproductive, digestive or circulatory, or to any combination thereof. While not addressed herein, a jet injector for the self-administration in the home of plural drugs to treat comorbid conditions can be multi-nozzled to mate with complementary openings into corresponding surface port openings in a matching surface port, as will be described.

Moreover, under the control of a fully implanted timing module or microcontroller responsive to a prescription-program, small pumps, in some instances very small, and in some instances, allowing drug replenishment from the surface port to a “micropump for drug delivery with integrated storage and electrically controlled timing” (see, for example, Tandon, V., Kang. W. S., Robbins. T. A., Spencer, A. J., Kim, E. S., Mckenna, M. J., Kujawa, S. G., and 5 others 2016. “Microfabricated Reciprocating Micropump for Intracochlear Drug Delivery with Integrated Drug/Fluid Storage and Electronically Controlled Dosing,” Lab on a Chip16(5): 829-846; Cobo. A., Sheybani, R., Tu H., and Meng, E. 2016. “A Wireless Implantable Micropump for Chronic Drug Infusion Against Cancer,” Sensors and Actuators A, Physical 239:18-25; Meng, E. and Hoang, T. 2012. “MEMS [microelectromechanical system]-enabled Implantable Drug Infusion Pumps for Laboratory Animal Research, Preclinical, and Clinical Applications,” Advanced Drug Delivert Reviews 64(14): 1628-1638; Gensler, H., Sheybani. R., Li, P. Y., Mann, R, L., and Meng, E. 2012. “An Implantable MEMS Micropump System for Drug Delivery in Small Animals,” Biomedical Microdevices 14(3): 483-496; Meng. E., Li, P. Y., Lo. R., Sheybani, R., and Gutierrez, C. 2009. “Implantable MEMS Drug Delivery Pumps for Small Animal Research,” Conference Proceedings: IEEE [Institute of Electrical and Electronics Engineers] Engineering in Medicine and Biology Society 2009:6696-6698) at the outlets of subdermally positioned small flat reservoirs (see also, for example Staples, M. 2010. “Microchips and Controlled-release Drug Reservoirs,” Wiley Interdisciplinary Reviews. Nanomedicine and Nanobiotechnology 2(4): 400-417) at the back of the port into which injected drugs are delivered can be delivered along separate accessory channels to multiple side-entry jackets and nonjacketing side-entry connectors.

This allows the direct targeting of agents to image, diagnose, and/or treat comorbidities. While compatible with such pronounced miniaturization, most applications contemplated herein do not call for a micro or nano degree of miniaturization. The ductus side-entry jackets can be positioned at different levels along one ductus or to different ductus, while nonjacketing side-entry connectors are affixed to one organ or to several organs belonging to different organ systems, as described in U.S. Pat. No. 11,759,186, entitled Ductus Side-entry Jackets and Prosthetic Disorder Response Systems, and U.S. Pat. No. 11,013,858, entitled Nonjacketing Side-entry Connectors and Prosthetic Disorder Response Systems. When the drugs or other agents are radioactive, depending upon their dose rate and latency, the accessory channel and nonjacketing side-entry connector are radiation shielded as shown in the same two patents.

This data can then be applied in accordance with an implanted prescription-programed microcontroller to adjust drug delivery to any of a number of locations automatically in a fully implanted and ambulatory feedback system. The result is that the patient need only replenish the set of subcutaneously (subdermally) implanted flat drug reservoirs periodically, and—when all accessory channel openings of the body surface port are incurrent and subcutaneous-replenish the power of the implanted rechargeable battery by transcutaneous energy transfer. Otherwise, the patient is oblivious to the automatic diagnostics and direct piping of drugs responsive to his disorder or disorders as he goes about his daily activities.

A body surface port is essentially a specially adapted nonjacketing side-entry connector as addressed in U.S. Pat. No. 11,013,858, entitled Nonjacketing Side-entry Connectors and Prosthetic Disorder Response Systems, usually placed in the pectoral region, as would a portacath chest port or totally implantable venous access device, or in the upper arm. The direct delivery to a diseased ureter is accomplished by positioning a ductus side-entry jacket above the lesion. When the ureter might be salvaged through remedial measures, so that temporary diversion to the contralateral ureter or to the bladder would allow time for healing, the adjustable ductus side-entry diversion jacket chute is adjusted to allow medication to allow the bulk of urine to be diverted while a lesser volume is allowed to flow down into the diseased segment.

The recipient-to-donor solid organ circulatory system sudden compound vascular bypass, or sudden switched-organ procedure, to include heart transplantation to be described, might be characterized as a form of cross-circulation, but only in a loosely analogous sense. Cross-circulation began in Belgium in 1890 when physiologist L. Frederic moved blood between the carotid arteries of two dogs to accomplish shared circulation and thereby blend the blood-bome immunological constituents of the two.

The controlled, or pump-assisted, cross-circulation demonstrated in 1954 by Clarence Walton Lillehei, Morley Cohen, Herbert Warden, and Richard Varco to circumvent the heart of the patient used cannulae and transparent plastic tubes to move blood between a volunteer and patient (Cooley, D. A 1999. “C. Walton Lillehei, the “Father of Open Heart Surgery,” Online, Circulation 100(13): 1364-1365; Cooley, D. A 1999. “A Tribute to C. Walton Lillehei, the “Father of Open Heart Surgery,” Online, Texas Heart Institute Journal 26(3): 165-166; Shumway, N, E. 1999. “C. Walton and F. John,” Annals of Thoracic Surgery 68(3 Supplement): S34-S36; Lillehei, C. W. 1955. “Controlled Cross-circulation for Direct-vision Intracardiac Surgery: Correction of Ventricular Septal Defects, Atrioventricularis Communis, and Tetralogy of Fallot,” Postgraduate Medicine 17(5): 388-396).

More than one combination of vessels in use at the time, in the Lillehei operation, one cannula was inserted remotely from the heart into the femoral artery of the volunteer, whose blood was passed through clear plastic tubing through a pump and from the pump through tubing through a cannula inserted into the aortic arch of the patient. The venae cavae of the patient were ligated, his desaturated blood sent through tubing to the great saphenous vein and lungs of the volunteer for oxygenation and return to the patient, whose azygos vein served in lieu of his venae cavae to return blood to his heart at 10 percent the normal rate. Except for the juxtapositioning and exchange of blood between two subjects, instant switching between the circulatory systems of a brain-dead donor on life support and a recipient shares nothing with the Lillehei procedure, which sought to allow intracardiac access for open heart (not just open chest) surgery, and not solid organ transplantation.

The procedure arose instead as but one application for ductus side-entry diversion jacket electrovalves, which are no less applicable anywhere else along the vascular tree or the urinary tract—not the reverse, not by development of a device for the limited purpose of solid organ, here, heart transplantation. Unless volunteer and patient differ markedly in size or weight and the recipient does not present significant pulmonary vascular resistance, the load on both hearts is substantially constant. Such an approach to solid organ transplantation is made possible by the availability of ductus side-entry diversion jacket electrovalves, which did not exist for Lillehei.

In contrast to historical cross-circulation, the technique represented herein, reciprocal cross-circulation:

1. Does not place a volunteer in jeopardy.

2. Does not bypass the heart as is necessary to perform a conventional heart transplant, involves zero cardioplegia and zero ischemia time.

3. Compared to a conventional transplant or intricate surgical procedure to temporarily palliate a complex congenital defect; is accomplished in relatively little time.

4. Demands far less skill than conventional transplantation or reconstruction, allowing application in other than tertiary medical centers, and

5. Does not require the heart of the volunteer to support the bodies of both, severely limiting the size and weight of the patient to about 86 pounds, but rather suddenly switches the heart of the donor from his own machine-supported circulatory system to that of the recipient.

The relatively quick exchanging of one heart for another can replace the reconstruction of a complex congenital defect; the long-term outcome with a good if immunosuppressed heart is better, and the jackets used allow the immunosuppressant, or antirejection, medication to be kept to the minimum. Not only is ischemic time zero but procedural time a fraction of that required for repair by a pediatric cardiac surgeon. As previously stressed, compound vascular bypass, of switch technique heart transplantation avoids the need for cardiopulmonary bypass, aortic cross-clamping, and by allowing the use of regional rather than general anesthesia, reduces postoperative cognitive impairment, decline in ventricular function, perfusion defects, and myocardial deformation.

It also and myocardial deformation to the extent that such consequences, which typically resolve within a year following transplantation, are attributable to the use of general anesthesia rather than the extensive incision involved in non-switch methods of heart transplantation (Godown, J., Dodd, D. A., Stanley, M., Havens, C., Xu, M., and 3 others 2018. “Changes in Left Ventricular Strain Parameters following Pediatric Heart Transplantation,” Pediatric Transplantation 22(5): e13166; D'Andrea, A., De Rimini, M, L., America, R., Cirillo, C.l, Riegler, L., and 9 others 2017. “Association between Left Ventricular Perfusion Defects and Myocardial Deformation Indexes in Heart Transplantation Recipients,” Echocardiography 34(10): 1540-1543; Chinali, M., Esposito, C., Grutter, G., Iacobelli, R., Toscano, A., and 6 others 2017. “Cardiac Dysfunction in Children and Young Adults with Heart Transplantation: A Comprehensive Echocardiography Study,” Journal of Heart and Lung Transplantation (New York, New York) 36(5): 559-566; Grotenhuis, H. B., Nyns, E, C. A., Kantor, P. F., Dipchand, A. I., Greenway, S. C., and 4 others 2017. “Abnormal Myocardial Contractility after Pediatric Heart Transplantation by Cardiac MRI,” Pediatric Cardiology 38(6): 1198-1205; Elhoff, J. J., Chowdhury, S. M., Taylor, C, L., Hassid, M., Savage, A. J., Atz, A. M., and Butts, R. J. 2016. “Decline in Ventricular Function as a Result of General Anesthesia in Pediatric Heart Transplant Recipients,” Pediatric Transplantation 20(8): 1106-1110; Parthiban, A., Li, L., Kindel, S., Shirali, G., Roessner, B., and 5 others 2015. “Mechanical Dyssynchrony and Abnormal Regional Strain Promote Erroneous Measurement of Systolic Function in Pediatric Heart Transplantation,” Journal of the American Society of Echocardiography (St. Louis, Missouri) 28(10): 1161-1170, e2; McNamara, D., Di Salvo, T., Mathier, M., Keck, S., Semigran, M., and Dec, G. W. 1996. “Left Ventricular Dysfunction after Heart Transplantation: Incidence and Role of Enhanced Immunosuppression,” Journal of Heart and Lung Transplantation (New York, New York) 15(5): 506-515; additional references cited below in section 2a(2), entitled Inventive Content-pertinent Fetal Diagnostics).

Orthotopic heart transplantation in the face of preexisting malformity is rendered more flexible as to precise positioning in the chest if, as side-entry diversion jackets make possible, shunting can be substituted for direct anastomosis (see, for example, Harris, C., Cao, C., Croce, B., and Munkholm-Larsen, S. 2018, Op cit. section 2a; Lang, S. M., Frazier, E. A., Collins, R. T. 2nd. 2016. “Aortic Complications Following Pediatric Heart Transplantation: A Case Series and Review,” Annals of Pediatric Cardiology 9(1): 42-45; Hasegawa, T., Oshima, Y., Maruo, A., Matsuhisa, H., Tanaka, A., Noda, R., and Matsushima, S. 2015. “Aortic Arch Geometry after the Norwood Procedure: The Value of Arch Angle Augmentation,” Journal of Thoracic and Cardiovascular Surgery 150(2): 358-366; Lee, J. H., Park, E. A., Lee, W., Cho, H. J., Kim, K. B., and Hwang, H. Y. 2014. “Heart Transplantation in a Patient with Persistent Left Superior Vena Cava,” Korean Journal of Thoracic and Cardiovascular Surgery 47(6): 533-535; Neragi-Miandoab, S., Baran, D., Godelman, A., and Goldstein, D. J. 2014. “Orthotopic Heart Transplantation in Patients with Persistent Left Superior Vena Cava: Bicaval and Biatrial Techniques,” Annals of Thoracic Surgery 97(3): 1085-1087; Weymann, A., Schmack, B., Karck, M., and Szabo, G. 2012. “Orthotopic Heart Transplantation in a Patient with Persistent Left Superior Vena Cava,” Annals of Thoracic and Cardiovascular Surgeryl8(6): 554-556; Kanter, K. R., Mahle, W. T., Vincent, R. N., and Berg, A. M. 2011. “Aortic Complications after Pediatric Cardiac Transplantation in Patients with a Previous Norwood Reconstruction,” Seminars in Thoracic and Cardiovascular Surgery. Pediatric Cardiac Surgery Annual 14(1): 24-28; Sue, S. H., Wei, J., Chuang, Y. C., Chang, C. Y., Lee, W. C., and Lee, S. L. 2008. “Cardiac Transplantation for Congenitally Corrected Transposition of the Great Arteries: A Case Report,” Transplantation Proceedings 40(8): 2844-2845; Blanche, C., Valenza, M., Czer, L. S., and Trento, A. 1994. “Heart Transplantation in Corrected Transposition of the Great Arteries,” Journal of Heart and Lung Transplantation (New York, New York) 13(4): 631-634; Jebara, V. A., Dreyfus, G., Acar, C., Deloche, A., Couetil, J. P., Fabiani, J. N., and Carpentier, A. 1990. “Heart Transplantation for Corrected Transposition of the Great Vessels,” Journal of Cardiac Surgery 5(2): 102-105). Heterotaxy (heterotaxia, isomerism, situs ambiguous, situs ambiguus) which affects the development of the atria, is addressed above in this section.)

Implanted with the bloodlines used to connect corresponding vessels, the vessels of the donor and recipient do not meet at a flush end-to-end anastomosis but rather as connected at either end to an intervening catheteric segment. Nevertheless, stimulated by somatotropin, or human growth hormone, direct anastomoses of corresponding donor and recipient vessels or connection through an interposed segment in a fetus or neonate should not make a significant difference for growth. However, even where the synthetic segment is interposed along one and the same vessel in order to elongate it, or interposed with a tissue engineered or autologous graft, much less across an immune-significant junction bridged by a synthetic segment, the effect on growth in an infant if any is not addressed in the literature.

If necessary, the hormone can be supplemented (see, for example, Chaudhry, S. G., Bentley-Hibbert, S., Stern, J., Lobritto, S., Martinez, M., and 7 others 2018. “Growth of Liver Allografts over Time in Pediatric Transplant Recipients,” Online, Pediatric Transplantation (Copenhagen, Denmark) 22(2): 10.1111/petr.13104; Fine, R. N. and Stablein, D. 2005. “Long-term Use of Recombinant Human Growth Hormone in Pediatric Allograft Recipients: A Report of the NAPRTCS [North American Pediatric Renal Trials and Collaborative Studies] Transplant Registry,” Pediatric Nephrology (Berlin, Germany) 20(3): 404-408).

Tissue engineered grafts made of a woven fabric of poly-1-lactide acid and a 50:50 poly(1-lactic co-e caprolactone) copolymer seeded with autologous bone marrow mononuclear cells similarly grow in children, albeit with an occasional case will require follow-up need for angioplasty (see, for example, Sugiura, T., Matsumura, G., Miyamoto, S., Miyachi, H., Breuer, C. K., and Shinoka, T. 2018. “Tissue-engineered Vascular Grafts in Children with Congenital Heart Disease: Intermediate Term Follow-up,” Online, Seminars in Thoracic and Cardiovascular Surgery (Philadelphia, Pennsylvania) 30(2): 175-179).

### Backup Path to Protect Against Flow Obstruction of Bloodlines Traversing the Waist Following Compound Heterotopic Bypass Transplantation of a Second Heart in the Lower Abdomen

The heterotopic placement of a second heart is addressed below in section 2b(1)(b)(b5), entitled Heterotopic Double Heart Metered Compound Vascular Bypass, or Switched-organ Transplantation [an so on]. The preferred position for a second heart is in the chest to the right of the native or previously transplanted heart. However, numerous conditions can make placement in the chest inadvisable if not impossible. In that case, the next position to consider is in the lower abdomen. Placed there, any inadequacy of protection by the rib cage necessitates the use of a heart cage as described below in section 2b(1)(b)(b6), entitled Heart Cage for the Infrarenal Abdominal Positioning of a Second Heart.

Various schemes for placing a heart in the lower abdomen are addressed below in sections 2b(1)(b)(b16), entitled Direct Aortocaval Connection: Unified Entry Arterial and Exit Venous Confluence Conduits for the Heterotopic Connection of a Graft Heart in the Lower Abdomen, 2b(1)(b)(b17), entitled Doubly Heterotopic Double Heart Compound Vascular Bypass, or Switched-organ Transplantation, and 2b(1)(b)(b18), entitled Hybrid Doubly Heterotopic Double Heart Transplantation.

Confluence conduits are addressed early above in this section as well as in sections 2b(1)(b)(b14), entitled Confluence Conduits: Connection of One or More Bloodlines to Flow through a Common Channel to the Same or a Different Number of Bloodlines with Self-leveling Apportionment of Outflow, and 2b(1)(b)(b15), entitled Switchable Lines and Confluence Conduits: Long when Intercorporeal, to Short when Intracorporeal, Both Affording Connection of One or More Bloodlines to the Same or a Different Number of Bloodlines with Self-leveling Apportionment of Outflow.

From a surgical standpoint, in that only a small segment of the small intestine might need to be excised in order make room for a heart rather than a portion of the right lung, placement in the lower abdomen is less traumatizing. However, from a physiological standpoint, placement in the lower abdomen has certain shortcomings to include the resistance to the return flow of arterial blood to the heart of gravity and the passage of bloodlines past the waist where flexion and shifting over time could result in kinking, effectively synonymous with a myocardial infarction.

Sufficient pressure is readily obtained through the placement of bloodline-intraluminal inline axial pumps seen as part number 167 in the drawing figures. Small axial inline pumps can be positioned along larger gauged bloodlines, confluence conduits (combinatory or compound bloodlines), and druglines to propel, retard, withdraw, or aspirate the fluid passing through the line. Because these impose a power consumption burden, their use is suitable for time-limited procedures, such as in longer reciprocal cross-circulation switchable confluence conduit bloodlines used during a compound vascular bypass heart transplant.

For druglines, or sidelines, this may be necessary when the drug is gravity fed from an implanted drug reservoir usually placed subcutaneously in the pectoral region. When release from the reservoir is through a peristaltic pump-controlled reservoir outlet, an inline pump offers a way to alter the release after the fact. Such is essentially limited to situations where the practitioner chooses to manually override the implanted automatic controller or master control microprocessor prescription program.

In order to avert excessive flexion or kinking of the bloodlines, it is essential to dispel the risk associated with dependency upon single bloodlines. In some cases, allowing some slack will eliminate such risk, and if the routing of the bloodlines is such that such as risk does not exist, then the need for backup lines can be disregarded. Otherwise, redundancy is accomplished by providing a backup second bloodline following a different route to the same destination. Redundancy applies equally to arterial and venous bloodlines.

Obstruction of a bloodline traversing the waist is sensed by a thin film pressure sensor lining the bloodline providing intermittent data input to the master control microprocessor which automatically switches flow through the bypass. FIG. 15A depicts the half-way point in a compound vascular bypass orthotopic heart transplant where the valves will continue to retract until all blood flow to the recipient heart is cut off, the recipient heart then removed with the arterial and venous drainage vessel severed and clamped off. The same figure depicts the end point in vascular bypass heterotopic heart transplant where the recipient heart remains in place, so that the arterial and venous bloodlines to the right side will continue to supply the recipient heart so that blood is shared between the hearts.

When backup bloodlines are desired, sufficient donor outflow and venous inflow stumps are left to position the additional servovalves. The backup or secondary servovalves and bloodlines are positioned closely above the arterial and venous bloodlines. Should a primary bloodline become obstructed, the first command issued by the master control microprocessor is the release of heparin or a thrombolytic into the valve and bloodline to clear the obstruction if it is due to clot. If the obstruction is not cleared, a moment later the next command assumes a mechanical constriction or kinking in the bloodline so that the arterial servovalve on the left and the venous servovalve on the right are fully extended to pass all flow to the backup servovalves just beyond them which then channel the flow of blood through the backup bloodlines.

Never achieving physiological sufficiency and therefore allowing a progressive continuing hypoxia and degeneration throughout the body, provided a replacement heart is available, reconstruction or any other palliative measure should not be considered only to delay transplantation. The proper place for such methods is as a bridging strategy pending the availability of a heart, just as are assist devices and bilateral banding of the pulmonary arteries.

Side-entry diversion jackets, valves, and the lines connected to these are left in place following transplantation for their accessory channels to make possible the continued directly pipe-targeted release of medicinal and line maintenance fluids to the treatment site. Thereafter, unless the patient has a medical condition that recommends retaining the side-entry devices to allowing a longer period for the patient to fully recover, the vessels can be anastomosed. In the unusual circumstance that another heart will become necessary, these will already have been placed to expedite reoperation.

This applies to surgical techniques addressed below, as well as hybrid surgical/interventional methods (see, for example, Gupta, A. and Amin, Z. 2017. “Popular Hybrid Congenital Heart Procedures without Cardiopulmonary Bypass,” Online, Frontiers in Surgery 4:9; Bear, D. W. and Fleming, G, A. 2017. “Utilizing Hybrid Techniques to Maximize Clinical Outcomes in Congenital Heart Disease,” Current Cardiology Reports 19(8): 72; Agrawal. H., Alkashkari. W., and Kenny, D. 2017. “Evolution of Hybrid Interventions for Congenital Heart Disease,” Expert Review of Cardiovascular Therapy 15(4): 257-266; Gobergs. R., Salputra, E., and Lubaua. I. 2016, Op cit.; Greenleaf, C. E., Urencio, J. M., Salazar, J. D., and Dodge-Khatami, A. 2016. “Hypoplastic Left Heart Syndrome: Current Perspectives,” Translational Pediatrics 5(3): 142-147; Taqatqa, A., Diab, K. A., Stuart, C., Fogg, L., Ilbawi, M., and 6 others 2016. “Extended Application of the Hybrid Procedure in Neonates with Left-sided Obstructive Lesions in an Evolving Cardiac Program,” Pediatric Cardiology 37(3): 465-471; Yerebakan C., Valeske, K., Elmontaser, H., Yörüker, U., Mueller, M., and 7 others 2016. “Hybrid Therapy for Hypoplastic Left Heart Syndrome: Myth, Alternative, or Standard?,” Journal of Thoracic and Cardiovascular Surgery 151(4): 1112-1123.e1-e5: Talwar, S., Kamat, N. A., Choudhary, S. K., Ramakrishnan, S., Saxena, A., and 3 others 2016. “Mid-term Outcomes of Patients Undergoing Adjustable Pulmonary Artery Banding,” Indian Heart Journal 68(1): 72-76; Yerebakan, C., Murray, J., Valeske, K., Thul, J., Elmontaser, H., and 7 others 2015. “Long-term Results of Biventricular Repair after Initial Giessen Hybrid Approach for Hypoplastic Left Heart Variants,” Journal of Thoracic and Cardiovascular Surgery 149(4): 1112-1122.e2; Dave, H., Rosser, B., Knirsch, W., Hubler, M., Pretre, R., and Kretschmar, O. 2014. “Hybrid Approach for Hypoplastic Left Heart Syndrome and Its Variants: The Fate of the Pulmonary Arteries,” European Journal of Cardiothoracic Surgery 46(1): 14-19; Holzer, R. J. and Cheatham, J. P. 2013. “Hybrid Procedures in Congenital Heart Disease: Hypoplastic Left Heart Syndrome/Muscular Ventricular Septal Defect/Stenting of Pulmonary Arteries,” Interventional Cardiology Clinics 2(1): 23-38; Guleserian, K. J., Barker, G. M., Sharma, M. S., Macaluso, J., Huang. R., Nugent, A. W., and Forbess, J. M. 2013. Journal of Thoracic and Cardiovascular Surgery 145(1): 206-214; Photiadis, J., Sinzobahamvya, N., Hraska, V., and Asfour, B. 2012. “Does Bilateral Pulmonary Banding in Comparison to Norwood Procedure Improve Outcome in Neonates with Hypoplastic Left Heart Syndrome Beyond Second-stage Palliation? A Review of the Current Literature,” Thoracic and Cardiovascular Surgery 60(3): 181-188; see also below).

More generally, as immunosuppressant measures continue to improve, a technique of heart transplantation simpler, more flexible as to orthotopic positioning, and far more likely to succeed than any conventional technique is of value where the condition of the patient is urgent, especially where the patient is too distant to the closest quaternary medical center and/or where the inordinate expertise and skill required to reconstruct the malformed heart is lacking-such as an infant in a rural area with a single ventricle heart, a hypoplastic left heart demanding exigent repair that includes a mostly or completely closed off, that is, atresic, or atretic, aorta, or failed Norwood procedure, for example (see, for example, Alsoufi, B., Mahle, W. T., Manlhiot, C., Deshpande, S., Kogon, B., McCrindle, B. W., and Kanter, K. 2016. “Outcomes of Heart Transplantation in Children with Hypoplastic Left Heart Syndrome Previously Palliated with the Norwood Procedure,” Journal of Thoracic and Cardiovascular Surgery 151(1): 167-175.e1-e2; Lang, S. M., Frazier, E. A., Collins, R. T. 2nd. 2016, Op cit.; Alsoufi, B., Deshpande, S., McCracken, C., Kogon, B., Vincent. R., Mahle, W., and Kanter, K. 2015. “Results of Heart Transplantation Following Failed Staged Palliation of Hypoplastic Left Heart Syndrome and Related Single Ventricle Anomalies,” European Journal of Cardiothoracic Surgery 48(5): 792-799; Kanter, K. R., Mahle, W. T., Vincent, R. N., and Berg, A. M. 2011, Op cit.; Jacobs, J. P., Quintessenza, J. A., Chai, P. J., Lindberg, H, L., Asante- Korang. A., and 3 others 2006. “Rescue Cardiac Transplantation for Failing Staged Palliation in Patients with Hypoplastic Left Heart Syndrome,” Cardiology in the Young (Cambridge, England) 16(6): 556-562).

In contrast with heart transplantation in adults, most often done in response to end-stage heart failure, in pediatric cardiac surgical practice, transplantation, especially if expedited by new means, can serve in lieu of attempting the repair a heart so malformed that even an optimal correction would not likely prove durable (see, for example, Choi, H. M., Park, M. S., and Youn, J, C. 2019. Op cit. section 2a; Murala, J. and Si. M. S. 2017, Op cit. below under section (b2), entitled Antepartum Compound vascular bypass, or sudden switched-organ Transplantation of the Heart; Kirklin. J. K. 2015. “Advances in Mechanical Assist Devices and Artificial Hearts for Children,” Current Opinion in Pediatrics 27(5): 597-603; Schweiger, M., Stiasny, B., Dave, H., Cavigelli- Brunner, A., Balmer, C., and 4 others 2015. “Pediatric Heart Transplantation,” Journal of Thoracic Disease 7(3): 552-559; Shillingford, M., Ceithaml. E., and Bleiweis, M. 2013. Considerations in the Management of Hypoplastic Left Heart Syndrome,” Seminars in Cardiothoracic and Vascular Anesthesia 17(2): 128-136; Alsoufi, B., Manlhiot, C., Awan, A., Alfadley, F., Al-Ahmadi, M., and 3 others 2012. “Current Outcomes of the Glenn Bidirectional Cavopulmonary Connection for Single Ventricle Palliation,” European Journal of Cardiothoracic Surgery 42(1): 42-49; Kirklin, J. K., Pearce, F. B., Dabal, R. J., Carlo, W. Jr., and McGiffin, D, C. 2012. “Cardiac Transplantation and Mechanical Support for Functional Single Ventricle,” World Journal for Pediatric and Congenital Heart Surgery 3(2): 183-193; Rocha-e-Silva, R. 2012. “A New Technique for the Correction [sic-palliation] of Hypoplastic Left Heart Syndrome,” Clinics (Sao Paulo) 67(5): 521-524; Kulkami, S. and Rao. S. 2012. “Definitive Therapy for Hypoplastic Left Heart Syndrome-Indian Scenario,” Indian Heart Journal 64(4): 338-340; Sian Pincott, E. and Burch, M. 2011. “Indications for Heart Transplantation in Congenital Heart Disease,” Current Cardiology Reviews 7(2): 51-58; Michielon, G., Parisi, F., Squitieri, C., Carotti, A., Gagliardi, G., Pasquini, L., and Di Donato, R. M. 2003. “Orthotopic Heart Transplantation for Congenital Heart Disease: An Alternative for High-risk Fontan Candidates?,” Circulation 108 Supplement 1 1:11140-11149).

When a complex reconstruction fails so that transplantation becomes advisable, a technique for transplantation which is simpler, avoids hypoxia, and involves less dissection and procedural time, is less fraught with risk. A failing Fontan results in pulmonary arterial hypotension with inadequate cardiac output, caval hypertension, progressive heart failure, proinflammatory state, hepatic dysfunction, coagulative aberrations, such as shear-stress induced acquired von Willebrand syndrome, and poor quality of life.

Such applies to the two-stage Fontan repair of a univentricular or double inlet left ventricle heart in a child, which, primarily by altering blood flow, induces progressive heart failure and therewith, increasing damage to the digestive and other organ systems, and until recently, adversely affected remediation by heart transplantation (see, for example, Meinel. K., Korak, F., Dusleag. M., Strini, T., Baumgartner, D., and 5 others 2023. “Mild Acquired von Willebrand Syndrome and Cholestasis in Pediatric and Adult Patents with Fontan Circulation,” Online, MDPI [Multidisciplinary Digital Publishing Institute] Journal of Clinical Medicine (Basel, Switzerland) 12(3): 1240′ Perrin, N. Dore. A., van de Bruaene, A., Mongeon, F. P., Mondesert, B., and 5 others 2022. “The Fontan Circulation: From Ideal to Failing Hemodynamis and Drug Therapies for Optimization,” Canadian Journal of Cardiology 38(7): 1059-1071; Kumar, T. K. S. 2021. “The Failing Fontan,” Online, Indian Journal of Thoracic and Cardiovascular Surgery (New Delhi) 37 (Supplement 1): 82-90; Mazza, G. A., Gribaudo, E., and Agnoletti, G. 2021. “The Pathophysiology and Complications of Fontan Circulation,” Online, Acta Biomedica: Atenei Parmensis (Parma, Italy) 92(5): e2021260; Sharma, V. J., Iyengar, A. J., Zannino, D., Gentles. T., Justo, R., and 7 others 2021. “Protein-losing Enteropathy and Plastic Bronchitis after the Fontan Procedure,” Journal of Thoracic and Cardiovascular Surgery 161(6): 2158-2165; Icheva. V., Nowak-Machen, M., Budde, U., Jaschonek, K., Neunhoeffer. F., and 4 others 2018. “Acquired von Willebrand Syndrome in Congenital Heart Disease Surgery: Results from an Observational Case Series,” Online, Journal of Thrombosis and Haemostasis 16(11): 2150-2158; Peyton, C. 2018. “Protein-losing Enteropathy and Plastic Bronchitis after the Fontan Operation,” Online, Critical Care Nurse 38(6): e5-e12; Simpson, K. E., Pruitt, E., Kirklin, J. K., Naftel, D. C., Singh, R. K., and 3 others 2017. “Fontan Patient Survival after Pediatric Heart Transplantation Has Improved in the Current Era,” Annals of Thoracic Surgery 103(4): 1315-1320; Caneo, L. F., Neirotti, R. A., Turquetto, A, L., and Jatene, M, B. 2016. “The Fontan Operation is Not the End of the Road,” (in English and Portuguese) Arquivos Brasileiros Cardiologia 106(2): 162-165; Gewillig, M. and Goldberg, D. J. 2014. “Failure of the Fontan Circulation,” Heart Failure Clinics 10(1): 105-116; Loeffelbein, F., Funk, D., Nakamura, L., Zieger. B., Grohmann, J., and 3 others 2014. “Shear-stress Induced Acquired von Willebrand Syndrome in Children with Congenital Heart Disease,” Online, Interactive Cardiovascular and Thoracic Surgery 19(6): 926-932; Sanders, D. B., Sowell, S. R., Park, S. S., Derby. C., Willis, B. C., and 5 others 2014. “The Failing Fontan: What's NEXT . . . ?,” Perfusion 29(1): 89-93; Goldberg, D. J., Shaddy, R. E., Ravishankar. C., and Rychik, J. 2011. “The Failing Fontan: Etiology, Diagnosis and Management,” Expert Review of Cardiovascular Therapy 9(6): 785-793; Michielon, G., Parisi. F., Di Carlo, D., Squitieri, C., Carotti, A., Buratta, M., and Di Donato, R. M. 2003. “Orthotopic Heart Transplantation for Failing Single Ventricle Physiology,” European Journal of Cardiothoracic Surgery 24(4): 502-510; Michielon, G., Carotti, A., Pongiglione, G., Cogo, P., and Parisi, F. 2011. “Orthotopic Heart Transplantation in Patients with Univentricular Physiology,” Current Cardiology Reviews 7(2): 85-91; U.S. Pat. No. 11,389,171, entitled Integrated System for the Infixion and Retrieval of Implants, provides additional references pertaining to failing Fontan circulation under Background of the Invention). When primary, the postnatal association of anomalies of the heart with anomalies in other organs is congenital, and if not interdicted, will result in maldevelopment of the brain.

If not corrected, a failing Fontan leads to irreversible multiple organ damage which may necessitate that those other organs be replaced as well (see, for example, Hill, K. D., Sampson, M. R., Li. J. S., Tunks, R. D., Schulman, S. R., and Cohen-Wolkowiez, M. 2016. “Pharmacokinetics of Intravenous Sildenafil in Children with Palliated Single Ventricle Heart Defects: Effect of Elevated Hepatic Pressures,” Cardiology in the Young (Cambridge, England) 26(2): 354-362; Alsoufi, B., McCracken, C., Schlosser, B., Sachdeva, R., Well. A., and 3 others 2016. “Outcomes of Multistage Palliation of Infants with Functional Single Ventricle and Heterotaxy Syndrome,” Journal of Thoracic and Cardiovascular Surgery 151(5): 1369-1377.e2; Peixoto, A. B., Tonni, G., and Araujo Junior, E. 2016. “Changes in Biometry and Cerebroplacental Hemodynamics in Fetuses with Congenital Heart Diseases,” Journal of Thoracic Disease 8(10): E1282-E1286; Como, A, F. 2015. “Editorial: Univentricular Heart,” Online, Frontiers in Pediatrics 3:75; Davies, R, R. and Pizarro, C. 2015. “Decision-making for Surgery in the Management of Patients with Univentricular Heart,” Online, Frontiers in Pediatrics 3:61; Alsoufi, B., Manlhiot, C., Ehrlich, A., Oster, M., Kogon, B., and 4 others 2015. “Results of Palliation with an Initial Pulmonary Artery Band in Patients with Single Ventricle Associated with Unrestricted Pulmonary Blood Flow,” Journal of Thoracic and Cardiovascular Surgery 149(1): 213-220; Gewillig, M. and Goldberg, D. J. 2014, Op cit.; Iyengar, A. J., Sharma, V. J., Weintraub, R. G., Shipp. A., Brizard, C. P., d'Udekem, Y., and Konstantinov, I, E. 2014. “Surgical Strategies to Facilitate Heart Transplantation in Children after Failed Univentricular Palliations: The Role of Advanced Intraoperative Surgical Preparation,” European Journal of Cardiothoracic Surgery 46(3): 480-485; Deshpande, S, R. and Maher, K. O. 2014. “Long-term Negative Pressure Ventilation: Rescue for the Failing Fontan?,” World Journal of Cardiology 6(8): 861-864; Goldberg, D. J., Shaddy, R. E., Ravishankar, C., and Rychik, J. 2011, Op cit.). Hence, where an original repair is survivable but would result in abnormal circulation that would lead to such a consequence, whenever possible, transplantation as the first response is clearly preferable to allowing such degeneration to progress.

The nonpulsatile circulation following a Fontan known to be unhealthy, the use of mechanical assist devices which perpetuate a pulseless circulation to include ventricular assist devices and cavopulmonary pumps are helpful for a time, but less and less adequate over time, making their use as a bridge to organ transplantation useful (see, for example, Rodefeld, M. D., Marsden, A., Figliola, R., Jonas. T., Neary. M., and Giridharan, G, A. 2019. “Cavopulmonary Assist: Long-term Reversal of the Fontan Paradox,” Online, Journal of Thoracic and Cardiovascular Surgery 158(6): 1627-1636). Ventricular assist devices excessively increase the blood pressure in Fontan circulation (Yang. W., Conover, T., Giridharan, G., Figliola, R., Marsden, A., and Rodenfeldf. M. 2023. “Passive Performance Evaluation and Validation of a Viscous Impeller Pump for Subpulmonary Fontan Circulatory Support,” Online, Research Square Preprint).

Much the same may be said for the first of the three-stages to repair a hypoplastic left heart, or Norwood procedure, where a univentricular heart, which experience shows remains functional over an interval limited by growth, is created. Even the most recent hybrid procedures do not yield acceptable development, health, or longevity (Chan, F. T., Bellsham-Revell, H. R., Duggan, H., Simpson, J. M., Hulse, T., and Bell, A. J. 2017. “Interstage Somatic Growth in Children with Hypoplastic Left Heart Syndrome after Initial Palliation with the Hybrid Procedure,” Cardiology in the Young (Cambridge, England) 27(1): 131-138; Ohye, R. G., Schranz, D., and D'Udekem, Y. 2016. “Current Therapy for Hypoplastic Left Heart Syndrome and Related Single Ventricle Lesions,” Circulation 134(17): 1265-1279).

Notwithstanding recent improvements (see, for example, Kenny, L. A., DeRita, F., Nassar, M., Dark, J., Coats, L., and Hasan, A. 2018. “Transplantation in the Single Ventricle Population,” Annals of Cardiothoracic Surgery (Hong Kong, China) 7(1): 152-159; Davies, R, R. and Pizarro, C. 2015. Op cit.), during the ensuing year following surgery, over one-quarter of these patients die or require a heart transplant, the best outcomes forestalling death no more than five years, and exceptionally, two or even three decades (see, for example, Alsoufi, B., Mahle, W. T., Manlhiot, C., Deshpande. S., Kogon. B., McCrindle, B. W., and Kanter, K. 2016, Op cit; Yabrodi, M. and Mastropietro, C. W. 2017. “Hypoplastic Left Heart Syndrome: From Comfort Care to Long-term Survival,” Pediatric Research 81(1-2): 142-149; Alsoufi, B., Deshpande, S., McCracken, C., Kogon, B., Vincent, R., Mahle. W., and Kanter, K. 2015, Op cit.; Gobergs, R., Salputra, E., and Lubaua, I. 2016; Op cit.; Greenleaf, C. E., Urencio, J. M., Salazar, J. D., and Dodge-Khatami, A. 2016. “Hypoplastic Left Heart Syndrome: Current Perspectives,” Translational Pediatrics 5(3): 142-147; Alsoufi, B., McCracken, C., Schlosser, B., Sachdeva, R., Well. A., and 3 others 2016. “Outcomes of Multistage Palliation of Infants with Functional Single Ventricle and Heterotaxy Syndrome,” Journal of Thoracic and Cardiovascular Surgery 151(5): 1369-1377.e2; Alsoufi, B., Slesnick, T., McCracken, C., Ehrlich, A., Kanter, K., and 4 others 2015. “Current outcomes of the Norwood Operation in Patients with Single-ventricle Malformations Other than Hypoplastic Left Heart Syndrome,” World Journal for Pediatric and Congenital Heart Surgery 6(1): 46-52; Alsoufi, B., Deshpande, S., McCracken, C., Kogon, B., Vincent. R., Mahle, W., and Kanter, K. 2015, Op cit.; Ugonabo, N., Hirsch-Romano, J. C., and Uzark, K. 2015. “The Role of Home Monitoring in Interstage Management of Infants following the Norwood Procedure,” World Journal for Pediatric and Congenital Heart Surgery 6(2): 266-273; Corno, A, F. 2015. “Functionally” Univentricular Hearts: Impact of Pre-natal Diagnosis,” Online, Frontiers in Pediatrics 3:15; Karamlou, T., Overman, D., Hill, K. D., Wallace, A., Pasquali, S. K., and 3 others 2015. “Stage 1 Hybrid Palliation for Hypoplastic Left Heart Syndrome-Assessment of Contemporary Patterns of Use: An Analysis of The Society of Thoracic Surgeons Congenital Heart Surgery Database,” Journal of Thoracic and Cardiovascular Surgery 149(1): 195-202.e1; Amold, R. R., Loukanov. T., and Gorenflo, M. 2014. “Hypoplastic Left Heart Syndrome-Unresolved Issues,” Online, Frontiers in Pediatrics 2:125; Knirsch, W., Bertholdt. S., Stoffel, G., Stiasny, B., Weber, R., and 4 others 2014. “Clinical Course and Interstage Monitoring after the Norwood and Hybrid Procedures for Hypoplastic Left Heart Syndrome,” Pediatric Cardiology 35(5): 851-856; Rudd, N. A., Frommelt, M. A., Tweddell, J. S., Hehir, D. A., Mussatto, K. A., and 4 others 2014. “Improving Interstage Survival after Norwood Operation: Outcomes from 10 Years of Home Monitoring,” Journal of Thoracic and Cardiovascular Surgery 148(4): 1540-1547; Lloyd, D. F., Cutler, L., Tibby, S. M., Vimalesvaran, S., Qureshi, S. A., and 5 others 2014. “Analysis of Preoperative Condition and Interstage Mortality in Norwood and Hybrid Procedures for Hypoplastic Left Heart Syndrome Using the Aristotle Scoring System,” Heart 100(10): 775-780; Hansen, J. H., Petko, C., Bauer, G., Voges, I., Kramer, H. H., and Scheewe, J. 2012. “Fifteen-year Single-center Experience with the Norwood Operation for Complex Lesions with Single-ventricle Physiology Compared with Hypoplastic Left Heart Syndrome,” Journal of Thoracic and Cardiovascular Surgery 144(1): 166-172; Ghanayem, N. S., Allen, K. R., Tabbutt, S., Atz, A. M., Clabby, M, L., and 15 others with 147 collaborators 2012. “Interstage Mortality after the Norwood Procedure: Results of the Multicenter Single Ventricle Reconstruction Trial,” Journal of Thoracic and Cardiovascular Surgery 144(4): 896-906; Ohye, R. G., Schonbeck, J. V., Eghtesady, P., Laussen, P. C., Pizarro, C., and 13 others and 147 collaborators 2012. “Cause, Timing, and Location of Death in the Single Ventricle Reconstruction Trial,” Journal of Thoracic and Cardiovascular Surgery 144(4): 907-914; Sistino, J. J. and Bonilha, H. S. 2012. “Improvements in Survival and Neurodevelopmental Outcomes in Surgical Treatment of Hypoplastic Left Heart Syndrome: A Meta-analytic Review,” Journal of Extra-corporeal Technology 44(4): 216-223; Hornik, C. P., He, X., Jacobs, J. P., Li, J. S., Jaquiss, R. D., and 4 others 2011. “Complications after the Norwood Operation: An Analysis of The Society of Thoracic Surgeons Congenital Heart Surgery Database,” Annals of Thoracic Surgery 92(5): 1734-1740; Barron, D. J., Kilby, M. D., Davies, B., Wright, J. G., Jones, T. J., and Brawn, W. J. 2009. Op cit.; Stasik, C. N., Gelehrter, S., Goldberg, C. S., Bove, E, L., Devaney, E. J., and Ohye, R, G. 2006. “Current Outcomes and Risk Factors for the Norwood Procedure,” Journal of Thoracic and Cardiovascular Surgery 131(2): 412-417).

Despite the advancements made in preventing continued damage of congenital malformities which can be repaired transluminally in utero, for the foreseeable future, repair other than a septal defect or valve will have to wait until delivery (see, for example, Tulzer, A., Arzt, W., Gitter, R., Prandstetter, C., Grohmann, E., Mair, R., and Tulzer, G. 2018. “Immediate Effects and Outcome of In-utero Pulmonary Valvuloplasty in Fetuses with Pulmonary Atresia with Intact Ventricular Septum or Critical Pulmonary Stenosis,” Ultrasound in Obstetrics and Gynecology 52(2): 230-237; Wohlmuth, C., Wertaschnigg. D., Wieser, I., Arzt. W., and Tulzer, G. 2016. “Tissue Doppler Imaging in Fetuses with Aortic Stenosis and Evolving Hypoplastic Left Heart Syndrome before and after Fetal Aortic Valvuloplasty,” Ultrasound in Obstetrics and Gynecology 47(5): 608-615; Freud, L, R. and Tworetzky, W. 2016. “Fetal Interventions for Congenital Heart Disease,” Current Opinion in Pediatrics 28(2): 156-162; Yuan, S. M. and Humuruola, G. 2016. “Fetal Cardiac Interventions: Clinical and Experimental Research,” (in English) Postepy Kardiol Interwencyjnej [Advances in Interventional Cardiology] (Poznan) 12(2): 99-107; Beroukhim, R. S., Gauvreau K., Benavidez, O. J., Baird C. W., LaFranchi. T., and Tworetzky. W. 2015. “Perinatal Outcome after Prenatal Diagnosis of Single-ventricle Cardiac Defects,” Ultrasound in Obstetrics and Gynecology 45(6): 657-663; Freud, L. R., McElhinney, D. B., Yuan, S. M. 2014. “Fetal Cardiac Interventions: An Update of Therapeutic Options,” Revista Brasileira de Cirurgia Cardiovascular [Brazilian Journal of Cardiovascular Surgery] 29(3): 388-395; Marshall, A. C., Marx, G. R., Friedman, K. G., and 8 others 2014. “Fetal Aortic Valvuloplasty for Evolving Hypoplastic Left Heart Syndrome: Postnatal Outcomes of the First 100 Patients,” Circulation 130(8): 638-645; Marantz, P. and Grinenco, S. 2015. “Fetal Intervention for Critical Aortic Stenosis: Advances, Research and Postnatal Follow-up,” Current Opinion in Cardiology 30(1): 89-94; McElhinney, D. B., Tworetzky, W., and Lock, J, E. 2010. “Current Status of Fetal Cardiac Intervention,” Circulation 121(10): 1256-1263; Gardiner, H. M. 2008. “In-utero Intervention for Severe Congenital Heart Disease,” Best Practice and Research. Clinical Obstetrics and Gynaecology 22(1): 49-61; McElhinney, D. B., Marshall, A. C., Wilkins-Haug, L. E., Brown, D. W., Benson, C. B., and 5 others 2009. “Predictors Technical Success and Postnatal Biventricular Outcome after in Utero Aortic Valvuloplasty for Aortic Stenosis with Evolving Hypoplastic Left Heart Syndrome,” Circulation 120(15): 1482-1490; Marshall, A. C., Levine, J., Morash, D., Silva, V., Lock, J. E., and 4 others 2008. “Results of in Utero Atrial Septoplasty in Fetuses with Hypoplastic Left Heart Syndrome,” Prenatal Diagnosis 28(11): 1023-1028; Makikallio, K., McElhinney, D., Levine, J. C., Marx, G. R., Colan, S. D., and 4 others 2006. “Fetal Aortic Valve Stenosis and the Evolution of Hypoplastic Left Heart Syndrome: Patient Selection for Fetal Intervention,” Circulation 113(11): 1401-1405; Saiki, Y. and Rebeyka, I. M. 2001. “Fetal Cardiac Intervention and Surgery,” Seminars in Thoracic and Cardiovascular Surgery. Pediatric Cardiac Surgery Annual 4:256-270; Hanley, F. L. 1994. “Fetal Cardiac Surgery,” Advances in Cardiac Surgery 5:47-74).

While the relations between the in utero development of the heart and brain continue to be elucidated, where magnetic resonance imaging of the fetus indicates that abnormal cerebral flow concomitant with focal white matter injury may be due to anomalous development of the heart, either palliative surgery in utero with the intention of replacing the heart promptly following delivery or an in utero heart transplant as soon as a suitable heart can be found is essential to save the brain from further maldevelopment (see, for example, van Nisselrooij, A, E, L., Jansen, F, A. R., van Geloven, N., Linskens, I. H., Pajkrt. E., and 6 others 2020. Op cit., above in section 2a(1), entitled Congenital Defects of the Heart and Other Organs: The Importance of Prompt Intervention; Morton, P. D., Korotcova, L., Lewis, B. K., Bhuvanendran, S., Ramachandra, S. D., and 7 others 2017. “Abnormal Neurogenesis and Cortical Growth in Congenital Heart Disease,” Science Translational Medicine 9(374), pii: eaah 7029; Morton, P. D., Ishibashi, N., and Jonas, R, A. 2017. “Neurodevelopmental Abnormalities and Congenital Heart Disease: Insights into Altered Brain Maturation,” Circulation Research 120(6): 960-977; Wong. A., Chavez, T., O'Neil, S., Votava-Smith, J., Miller, D., and 3 others 2017. “Synchronous Aberrant Cerebellar and Opercular Development in Fetuses and Neonates with Congenital Heart Disease: Correlation with Early Communicative Neurodevelopmental Outcomes, Initial Experience,” American Journal of Perinatology Reports 7(1): e17-e27; Harbison, A, L., Votava-Smith, J. K., Del Castillo, S., Kumar, S. R., Lee. V., and 6 others 2017. “Clinical Factors Associated with Cerebral Metabolism in Term Neonates with Congenital Heart Disease,” Journal of Pediatrics 183:67-73. e1; Scala, C., Familiari, A., Pinas, A., Papageorghiou, A. T., Bhide, A., Thilaganathan, B., and Khalil, A. 2017. “Perinatal and Long-term Outcomes in Fetuses Diagnosed with Isolated Unilateral Ventriculomegaly: Systematic Review and Meta-analysis,” Ultrasound in Obstetrics and Gynecology 49(4): 450-459; Ruiz, A., Cruz-Lemini, M., Masoller, N., Sanz-Cortes, M., Ferrer, Q., and 9 others 2017. “Longitudinal Changes in Fetal Biometry and Cerebroplacental Hemodynamics in Fetuses with Congenital Heart Disease,” Ultrasound in Obstetrics and Gynecology 49(3): 379-386; Khalil. A., Bennet, S., Thilaganathan, B., Paladini, D., Griffiths, P., and Carvalho, J. S. 2016. “Prevalence of Prenatal Brain Abnormalities in Fetuses with Congenital Heart Disease: A Systematic Review,” Ultrasound in Obstetrics and Gynecology 48(3): 296-307; Abu-Rustum, R. S., Ziade, M. F., Abu-Rustum, S. E., and Daou, L. S. 2016. “Are There Head Volume Alterations at 11 to 14 Weeks in Fetuses with Congenital Heart Defects? A First Trimester Case Series,” American Journal of Perinatology Reports 6(2): e232-e238; McKenney, S, L., Masoller, N., Sanz-Cortes, M., Crispi, F., Gomez, O., Bennasar, M., and 4 others 2016. “Mid-gestation Brain Doppler and Head Biometry in Fetuses with Congenital Heart Disease Predict Abnormal Brain Development at Birth,” Ultrasound in Obstetrics and Gynecology 47(1): 65-73; Mansouri, F. F., Everett, A. D., Graham, E. M., Burd, I., and Sekar, P. 2016. “Glial Fibrillary Acidic Protein as a Biomarker for Brain Injury in Neonatal CHD,” Cardiology in the Young (Cambridge, England) 26(7): 1282-1289; Jansen, F. A., van Zwet, E. W., Rijlaarsdam, M. E., Paikrt, E., van Velzen, C, L., and 7 others 2016. “Head Growth in Fetuses with Isolated Congenital Heart Defects: Lack of Influence of Aortic Arch Flow and Ascending Aorta Oxygen Saturation,” Ultrasound in Obstetrics and Gynecology 48(3): 357-364; Li, Y., Yin, S., Fang, J., Hua, Y., Wang, C., Mu, D., and Zhou, K. 2015. “Neurodevelopmental Delay with Critical Congenital Heart Disease is Mainly from Prenatal Injury Not Infant Cardiac Surgery: Current Evidence Based on a Meta-analysis of Functional Magnetic Resonance Imaging,” Ultrasound in Obstetrics and Gynecology 45(6): 639-648; Abdel Raheem, M. M. and Mohamed, W, A. 2012. “Impact of Congenital Heart Disease on Brain Development in Newborn Infants,” Annals of Pediatric Cardiology 5(1): 21-26; McQuillen, P. S. and Miller, S. P. 2010. “Congenital Heart Disease and Brain Development,” Annals of the New York Academy of Sciences 1184:68-86; Limperopoulos, C., Tworetzky, W., McElhinney, D. B., Newburger, J. W., Brown, D. W., and 9 others 2010. “Brain Volume and Metabolism in Fetuses with Congenital Heart Disease: Evaluation with Quantitative Magnetic Resonance Imaging and Spectroscopy,” Circulation 121(1): 26-33).

Procedures other than transplantation, or if pulmonary vascular resistance is present, a heart and lung transplant, result in defective circulation and therewith, progressive multiple organ damage which invariably leads to a sick life and premature death. The superiority over such staged radical procedures of a facilitated method for transplantation following the initial diagnosis is clear, and the preferability of transplantation as the initial response is all the clearer because however good the heart, the degradation in other organs that results when a single ventricle is left to drive both the systemic and the pulmonary circulation leaves transplantation with irreversibly impaired organs incapable of sustaining life much less good health for long.

Variants involving cell therapy are also documented (see, for example, Emani, S. M. and Del Nido, P. J. 2018. “Cell-based Therapy with Cardiosphere-derived Cardiocytes: A New Hope for Pediatric Patients with Single Ventricle Congenital Heart Disease?,” Online, Circulation Research (Baltimore, Maryland) 122(7): 916-917; Sano. T., Ousaka, D., Goto, T., Ishigami, S., Hirai, K., and 4 others 2018. “Impact of Cardiac Progenitor Cells on Heart Failure and Survival in Single Ventricle Congenital Heart Disease,” Online, Circulation Research (Baltimore, Maryland) 122(7): 994-1005; Bittle, G. J., Wehman, B., Karathanasis, S. K., and Kaushal, S. 2017. “Clinical Progress in Cell Therapy for Single Ventricle Congenital Heart Disease,” Circulation Research (Baltimore, Maryland) 120(7): 1060-1062; Mari, M. A., Cascudo, M. M., and Alchieri, J, C. 2016. Congenital Heart Disease and Impacts on Child Development,” Brazilian Journal of Cardiovascular Surgery 31(1): 31-37; Everitt, M. D., Boyle, G. J., Schechtman, K. B., Zheng. J., Bullock, E. A., and 5 others 2012. “Early Survival after Heart Transplant in Young Infants is Lowest after Failed Single-ventricle Palliation: A Multi-institutional Study,” Journal of Heart and Lung Transplantation (New York, New York) 31(5): 509-516; Polat. S., Okuyaz, C., Hallioğlu. O., Mert. E., and Makharoblidze, K. 2011. “Evaluation of Growth and Neurodevelopment in Children with Congenital Heart Disease,” Pediatrics International 53(3): 345-349; Alkhaldi, A., Chin, C., and Bernstein, D. 2006. “Pediatric Cardiac Transplantation,” Seminars in Pediatric Surgery 15(3): 188-198).

The use of cardiac progenitor cells apart from and in conjunction with transplantation in congenital heart disease has also been addressed and while offering some benefits, found to result in reduced cell viability and poor engraftment into the host tissue (Abe, J., Yamada, Y., and Harashima “H. 2018. “The Use of Cardiac Progenitor Cells for Transplantation in Congenital Heart Disease and an Innovative Strategy for Activating Mitochondrial Function in Such Cells,” Online, Journal of Thoracic Disease (Hong Kong, China) 10 (Supplement 18): S2119-S2121).

With the object of regenerating infarcted myocardium, cardiosphere-derived cell therapy, mesenchymal stem cell-derived exosomes, bone marrow aspirate concentrate, exosome-mediated regenerative cell-to-cell communications, dental pulp stem cells, and other agents for myocardial regeneration remain in development with numerous obstacles to practical implementation, primarily the deterioration in protein and cell survival, which an automatic delivery system can overcome by immediately responsive sensors and release of cell and protein biomaterials devised to achieve effective delivery. Protected agents must be administered at the prescribed interval, making automatic release by an implant system decisively more dependable than voluntary compliance, and delivery directly pipeline targeted to the coronary arteries supplying the infarcted portion of the myocardium is critically more efficient than nontargeted infusion into the venous system, and the coronaries end-arterial, the targeting is especially effective.

Provided clinical efficacy is established, these cells, autologous and therefore not arousing an immune reaction, administered by infusion, will be suitable for delivery by an implanted disorder control system placed to competently medicate a patient with comorbid disease who had experienced a myocardial infarction so that the loss of myocytes will not progress to heart failure before reaching end-stage, when only heart transplantation will allow survival. Since few if any hearts would still be needed for patients with end-stage heart failure, the ability to heal the infarcted heart and stop the ensuing degeneration into end-stage heart failure would effectively increase the supply of hearts available for transplant.

Such an approach has the potential to regenerate myocardial tissue and thus cure, not just palliate, the infarcted myocardium (see, for example Madonna, R., Van Laake, L. W., Botker, H. E., Davidson, S. M., De Caterina, R., and 16 others 2019. “ESC [European Society of Cardiology] Working Group on Cellular Biology of the Heart: Position Paper for Cardiovascular Research: Tissue Engineering Strategies Combined with Cell Therapies for Cardiac Repair in Ischaemic Heart Disease and Heart Failure,” Cardiovascular Research 115(3): 488-500; Ciullo, A., Biemmi, V., Milano, G., Bolis, S., Cervio. E., and 6 others 2019. “Exosomal Expression of CXCR4 [fusin, cluster of differentiation 184 alpha-chemokine receptor specific for stromal-derived-factor-1] Targets Cardioprotective Vesicles to Myocardial Infarction and Improves Outcome after Systemic Administration,” Online, MDPI [Multidisciplinary Digital Publishing Institute] International Journal of Molecular Sciences (Basel, Switzerland) 20(3). pii: E468; Gho, Y. S. and Lee, J. 2019. “Special Issue on the Role of Extracellular Vesicles in Human Diseases,” Experimental and Molecular Medicine 51(3): 34; Duan, P., Tan, J., Miao. Y., and Zhang, Q. 2019. “Potential Role of Exosomes in the Pathophysiology, Diagnosis, and Treatment of Hypoxic Diseases,” American Journal of Translational Research 11(3): 1184-1201; Yang, L., Zhu, J., Zhang, C., Wang, J., Yue, F., Jia, X., and Liu, H. 2019. “Stem Cell-derived Extracellular Vesicles for Myocardial Infarction: A Meta- analysis of Controlled Animal Studies,” Aging (Albany, New York) 11(4): 1129-1150; Huang, K., Hu. S., and Cheng, K. 2019. “A New Era of Cardiac Cell Therapy: Opportunities and Challenges,” Advanced Healthcare Materials 8(2): e1801011; Murphy, D. E., de Jong, O. G., Brouwer, M., Wood, M. J., Lavieu, G., Schiffelers, R. M., and Vader, P. 2019. “Extracellular Vesicle-based Therapeutics: Natural versus Engineered Targeting and Trafficking,” Experimental and Molecular Medicine 51(3): 32; Cheng. M., Yang, J., Zhao, X., Zhang. E., Zeng, Q., and 12 others 2019. “Circulating Myocardial MicroRNAs from Infarcted Hearts are Carried in Exosomes and Mobilise Bone Marrow Progenitor Cells,” Nature Communications (London, England) 10(1): 959; Rodriguez, A. M. and Yin, V. P. 2019. “Emerging Roles for Immune Cells and MicroRNAs in Modulating the Response to Cardiac Injury,” Journal of Cardiovascular Development and Disease 6(1). pii: E5; Zhang, Y., Liu, Y., Liu, H., and Tang, W, H. 2019. “Exosomes: Biogenesis, Biologic Function and Clinical Potential,” Cell and Bioscience 9:19; Pleva, L., Kukla, P., Vitkova, K., and Procházka, V. 2019. “Rationale and Design of a Prospective, Randomised Study of Retrograde Application of Bone Marrow Aspirate Concentrate (BMAC) through Coronary Sinus in Patients with Congestive Heart Failure of Ischemic Etiology (the RETRO Study),” Online, BioMed Central Cardiovascular Disorders 19(1): 32; Li, N., Rochette, L., Wu, Y., and Rosenblatt-Velin, N. 2019. “New Insights into the Role of Exosomes in the Heart after Myocardial Infarction,” Journal of Cardiovascular Translational Research 12(1): 18-27; Li, Y., Huo, C., Pan, T., Li, L., Jin, X., and 6 others 2019. “Systematic Review Regulatory Principles of Non-coding RNAs in Cardiovascular Diseases,” Briefings in Bioinformatics 20(1): 66-76; Vrtovec, B. and Bolli, R. 2019. “Potential Strategies for Clinical Translation of Repeated Cell Therapy,” Circulation Research 124(5): 690-692; Psarras, S., Beis, D., Nikouli, S., Tsikitis, M., and Capetanaki, Y. 2019. “Three in a Box: Understanding Cardiomyocyte, Fibroblast, and Innate Immune Cell Interactions to Orchestrate Cardiac Repair Processes,” Online, Frontiers in Cardiovascular Medicine 6:32; Yamada, Y., Nakamura-Yamada, S., Kusano, K., and Baba, S. 2019. “Clinical Potential and Current Progress of Dental Pulp Stem Cells for Various Systemic Diseases in Regenerative Medicine: A Concise Review,” Online, MDPI [Multidisciplinary Digital Publishing Institute] International Journal of Molecular Sciences (Basel, Switzerland) 20(5). pii: E1132; Tu. Y., Qiu, Y., Liu, L., Huang. T., Tang. H., and 5 others 2019. “miR-15a/15b [miR-15 microRNA precursor family] Cluster Modulates Survival of Mesenchymal Stem Cells to Improve Its Therapeutic Efficacy of Myocardial Infarction,” Journal of the American Heart Association (Oxford, England) 8(1): e010157; Cai, H., Wu, F. Y., Wang, Q, L., Xu. P., Mou, F. F., and 6 others 2019. “Self-assembling Peptide Modified with QHREDGS [an integrin-binding angiopoietin-1-derived peptide] as a Novel Delivery System for Mesenchymal Stem Cell Transplantation after Myocardial Infarction,” Federation of American Societies for Experimental Biology Journal April 10: fj201801768R R; Moghaddam, A. S., Afshari, J. T., Esmaeili, S. A., Saburi. E., Joneidi, Z., and Momtazi-Borojeni, A, A. 2019. “Cardioprotective microRNAs: Lessons from Stem Cell-derived Exosomal microRNAs to Treat Cardiovascular Disease,” Atherosclerosis March 23:285:1-9; Hu. C., Zhao, L., Wu. D., and Li, L. 2019. “Modulating Autophagy in Mesenchymal Stem Cells Effectively Protects against Hypoxia- or Ischemia-induced Injury,” Stem Cell Research and Therapy 10(1): 120; Yin, K., Wang, S., and Zhao, R, C. 2019. “Exosomes from Mesenchymal Stem/Stromal Cells: A New Therapeutic Paradigm,” Biomark Research 7:8; Xiao, C., Wang, K., Xu. Y., Hu, H., Zhang, N. and 14 others 2018. “Transplanted Mesenchymal Stem Cells Reduce Autophagic Flux in Infarcted Hearts via the Exosomal Transfer of miR-125b,” Circulation Research (Baltimore, Maryland) 123(5): 564-578; Andriolo, G., Provasi. E., Lo Cicero. V., Brambilla, A., Soncin, S., and 6 others 2018. “Exosomes from Human Cardiac Progenitor Cells for Therapeutic Applications: Development of a GMP-Grade Manufacturing Method” Online, Frontiers in Physiology 9:1169; Adamiak, M. and Sahoo, S. 2018. “Exosomes in Myocardial Repair: Advances and Challenges in the Development of Next-generation Therapeutics,” Molecular Therapy 26(7): 1635-1643; Gerisch, M., Smettan, J., Ebert. S., Athelogou, M., Brand-Saberi. B., and 4 others 2018. “Qualitative and Quantitative Analysis of Cardiac Progenitor Cells in Cases of Myocarditis and Cardiomyopathy,” Online, Frontiers in Genetics 9:72; Merryweather-Clarke, A. T., Cook, D., Lara, B. J., Hua. P., Repapi. E., and 3 others 2018. “Does Osteogenic Potential of Clonal Human Bone Marrow Mesenchymal Stem/Stromal Cells Correlate with Their Vascular Supportive Ability?,” Stem Cell Research and Therapy 9(1): 351; Marban E. 2018. “A Mechanistic Roadmap for the Clinical Application of Cardiac Cell Therapies,” Nature Biomedical Engineering 2(6): 353-361; Shanmuganathan, M., Vughs. J., Noseda, M., and Emanueli, C. 2018. “Exosomes: Basic Biology and Technological Advancements Suggesting their Potential as Ischemic Heart Disease Therapeutics,” Online, Frontiers in Physiology 9:1159; Hashimoto, H., Olson, E. N., and Bassel-Duby, R. 2018. “Therapeutic Approaches for Cardiac Regeneration and Repair,” Nature Reviews. Cardiology 15(10): 585-600; Wang. W. J., Wang, Y. M., Hu. Y., Lin, Q., Chen, R., and 6 others 2018. “HDncRNA [heart disease-related non-coding RNA]: A Comprehensive Database of Non-coding RNAs Associated with Heart Diseases,” Database (Oxford, England) 2018; Ashur, C. and Frishman, W, H. 2018. “Cardiosphere-derived Cells and Ischemic Heart Failure,” Cardiology in Review 26(1): 8-21; Ghiroldi, A., Piccoli, M., Cirillo, F., Monasky, M. M., Ciconte, G., Pappone, C., and Anastasia, L. 2018. “Cell-based Therapies for Cardiac Regeneration: A Comprehensive Review of Past and Ongoing Strategies,” Online, MDPI [Multidisciplinary Digital Publishing Institute] International Journal of Molecular Sciences (Basel, Switzerland) 19(10). pii: E3194; Das, A., Samidurai, A., and Salloum, F. N. 2018. “Deciphering Non-coding RNAs in Cardiovascular Health and Disease,” Online, Frontiers in Cardiovascular Medicine 5:73; Namazi, H., Namazi, I, Ghiasi, P., Ansari, H., Rajabi, S., and 3 others 2018. “Exosomes Secreted by Normoxic and Hypoxic Cardiosphere-derived Cells Have Anti-apoptotic Effect,” Online, Iranian Journal of Pharmaceutical Research 17(1): 377-385; Amini, H., Rezaie, J., Vosoughi, A., Rahbarghazi, R., and Nouri, M. 2017. “Cardiac Progenitor Cells Application in Cardiovascular Disease,” Journal of Cardiovascular and Thoracic Research 9(3): 127-132; Barile, L., Milano, G., and Vassalli, G. 2017. “Beneficial Effects of Exosomes Secreted by Cardiac-derived Progenitor Cells and Other Cell Types in Myocardial Ischemia,” Stem Cell Investigation 4:93; Harvey, E., Zhang, H., Sepulveda, P., Garcia, S. P., Sweeney, D., and 10 others 2017. “Potency of Human Cardiosphere-derived Cells from Patients with Ischemic Heart Disease is Associated with Robust Vascular Supportive Ability,” Stem Cells Translational Medicine 6(5): 1399-1411; Zhang, Y., Hu, Y. W., Zheng, L., and Wang, Q. 2017. “Characteristics and Roles of Exosomes in Cardiovascular Disease,” DNA and Cell Biology 36(3): 202-211; Shafei, A. E., Ali, M. A., Ghanem, H. G., Shehata, A. I., Abdelgawad, A. A., and 4 others 2017. “Mesenchymal Stem Cell Therapy: A Promising Cell-based Therapy for Treatment of Myocardial Infarction,” Journal of Gene Medicine 19(12); Yu, H., Lu, K., Zhu, J., and Wang, J. 2017. “Stem Cell Therapy for Ischemic Heart Diseases,” Online, British Medical Bulletin 121(1): 135-154; Katarzyna, R. 2017. “Adult Stem Cell Therapy for Cardiac Repair in Patients after Acute Myocardial Infarction Leading to Ischemic Heart Failure: An Overview of Evidence from the Recent Clinical Trials,” Current Cardiology Reviews 2017 13(3): 223-231; Safari, S., Malekvandfard, F., Babashah, S., Alizadehasl, A., Sadeghizadeh, M., and Motavaf, M. 2016. “Mesenchymal Stem Cell-derived Exosomes: A Novel Potential Therapeutic Avenue for Cardiac Regeneration,” Cellular and Molecular Biology (Noisy-le-Grand, France) 62(7): 66-73; Kapelios, C. J., Nanas, J. N., and Malliaras, K. 2016. “Allogeneic Cardiosphere-derived Cells for Myocardial Regeneration: Current Progress and Recent Results,” Future Cardiology 12(1): 87-100; Reich, H., Tseliou, E., de Couto, G., Angert, D., Valle, J., and 7 others 2016. “Repeated Transplantation of Allogeneic Cardiosphere-derived Cells Boosts Therapeutic Benefits without Immune Sensitization in a Rat Model of Myocardial Infarction,” Journal of Heart and Lung Transplantation (New York, New York) 35(11): 1348-1357; Sharma, S., Mishra, R., Simpson, D., Wehman, B., Colletti, E. J., and 8 others 2015. “Cardiosphere-derived Cells from Pediatric End-stage Heart Failure Patients Have Enhanced Functional Activity Due to the Heat Shock Response Regulating the Secretome,” Stem Cells (Dayton, Ohio) 33(4): 1213-1229; Afzal, M. R., Samanta, A., Shah, Z. I., Jeevanantham, V., Abdel-Latif, A., Zuba-Surma, E. K., and Dawn, B. 2015. “Adult Bone Marrow Cell Therapy for Ischemic Heart Disease: Evidence and Insights from Randomized Controlled Trials,” Circulation Research 117(6): 558-575; Pascual-Gil, S., Garbayo, E., Diaz-Herráez, P., Prosper, F., and Blanco-Prieto, M. J. 2015. “Heart Regeneration after Myocardial Infarction Using Synthetic Biomaterials,” Journal of Controlled Release 203:23-38).

As proposed, stem cell therapy may improve cardiac output pending the availability of a replacement heart and compensate for a temporary reduction in ventricular absolute stroke volume following transplantation. However, once mature, this technology can be expected to have applications beyond mending of the post-heart attack heart, post-transplantation support of the heart, for example, high on the list.

Adding a ledge or lip at the jacket outlet opening or ostium leading out of the native lumen and into the side connector can be used to change the proportion of flow tapped, but for any one such jacket this proportion will be fixed. By contrast, side-entry diversion valve-jackets are either bistable, that is, adjusted as to be fully open or fully closed with no portion of the flow tapped, or continuously variable to allow adjustment in the relative proportion of flow tapped. The mainlines to be described can either be removed following anastomosis of the transected vessels, or if the bypassed segment remains irreparable or vulnerable, can be left in place indefinitely. The mainlines are removed and the side connector capped off, the jackets with sidelines, or accessory channels, connected to the port at the body surface are left in place to target immunosuppressants and other medication directly to the anastomoses, sparing exposure of these to the rest of the body.

To be described, the three side-entry diversion jackets used as endovascular valves to create the bypass needed to perform a zero-ischemic-time carotid endarterectomy, the four used to correct a transposition of the great arteries, or ventriculoatrial discordance, and the additional jackets needed for the conotruncal (conotruncus, bulbus cordis)-derived vessels and venae cavae in a heart transplant are of the bistable type. That is, the valves are switched from completely closed to completely open or the reverse simultaneously from a switch operated by the operator or an assistant, the valves moved by tiny plunger solenoids.

Where the operator, even without the aid of an assistant, should be able to adjust the jackets in carotid and other endarterectomies, aneurysm repairs or bypasses, and transpositions quickly by hand, in heart transplantation and other more complex operations, the larger number of vascular valves needed are adjusted simultaneously, all the valve-jacket chutes moved at the same instant by damped push-pull solenoids actuated by the operator with a plunger switch. Side-entry diversion jackets of any type can be used to bypass blood during open repair or remain indefinitely as prostheses.

A circumtrepan vacuum jacket, or mantle, comprising an inner tubular layer with perforations separated by a thin space from an outer layer positioned just beneath the jacket shell connected through the mainline to the same aspiration pump as is used to aid cutting by the trepan of the ductus side entry hole acts to distribute the trepan vacuum to the adventitia, preventing gouging injury to the intima opposite the trepan used to remove a tissue plug from the side of the ductus. Thereafter, the vacuum jacket serves to target anti-inflammatory and other drugs directly to the jacket foam lining-adventitial interface through an accessory channel.

The foam jacket lining inside, that is, adluminal to the inner layer of the vacuum mantle is clear of the vacuum perforations, the vacuum therefore drawing the outer wall of the ductus outward to protect the nontrephined circumference of the ductus from gouging injury. For solid organ transplantation, basic side-entry and side-entry valve-jackets address both major obstacles to success, rejection through the direct targeting of immunosuppressants to the treatment site, sparing the exposure of other tissue, and the avoidance of direct-contact native-to allogeneic tissue anastomoses, which are susceptible to failure. Graft organs susceptible to the development of malignant tumors, the jackets are also prepositioned to directly target anticancer drugs to the affected sites.

### Access to Allow the Directly Targeted Treatment and Diagnosis of the Leptomeninges and Cerebrospinal Fluid

Side-entry devices to include vascular valves and servovalves perivascular, that is, positioned to encircle a substrate ductus, and nonjacketing side-entry connectors designed to be attached to a flat or curved surface, means for accomplishing direct access to the meninges and central nervous system remain to be specified.

Because of the risk posed in replacing a patch aortoplasty or a prosthetic thoracic aorta, and the pain of a thoracic reentry wound which follows once the anesthesia has worn off, the importance of avoiding the need for revisions cannot be exaggerated (see, for example, Peng, J., Wang, Z., Ma, L., Ma, W. Liu, G., and 4 others 2022. “Incidence and Influencing Factors of Chronic Postthoracotomy Pain in Lung Tumor Patients,” Journal of Healthcare Engineering 2022:7584481; Kar, P., Sudheshna, K. D., Padmaja, D., Pathy, A. and Gopnath, R. 2019. “Chronic Pain following Thoracotomy for Lung Surgeries: It's Risk Factors, Prevalence, and Impact on Quality of Life-A Retrospective Study,” Indian Journal of Anaesthesia 63(5): 368-374; Bayman, E. O., Parekh, K. R., Keech, J., Selte, A., and Brennan, T. J. 2017. “A Prospective Study of Chronic Pain after Thoracic Surgery,” Anesthesiology 126(5): 938-951; Bayman, E. O. and Brennan, T. J. 2014. “Incidence and Severity of Chronic Pain at 3 and 6 Months after Thoracotomy: Meta-analysis,” Journal of Pain 15(9): 887-897; Kinney, M, A. O, Hooten, W. M., Cassivi, S. D., Allen, M. S., Passe, M. A., and 3 others 2012. “Chronic Postthoracotomy Pain and Health-related Quality of Life,” Annals of Thoracic Surgery 93(4): 1242-1247; Katz, J., Jackson, M. Kavanagh, B. P. and Sandler, A. N. 1996. “Acute Pain after Thoracic Surgery Predicts Long-term Post-thoracotomy Pain,” Clinical Journal of Pain 12(1): 50-55). Such a prosthesis is no less applicable to replacement of an acquired defect such as a severe coarctation, interrupted aortic arch, or aneurysm in an adult.

The need for a prosthetic thoracic aorta, for example, to replace one congenitally malformed with a severe coarctation, interruption, or aneurysm, especially when due to a condition that could lead to the reformation of a severe defect at any time in the subsequent life of a neonate or infant was addressed above in section 2a, entitled Background of the Invention. General. To accommodate both the pulse and growth over a considerable if not life-long range from neonate to adult requires a novel expandable structure able to respond throughout this period not only to the pulse but to the stretching vectors associated with growth until upon reaching adulthood, an absolute limit to further extension where sufficient elasticity is still sufficient to accommodate the pulse is met at which further stretching is prevented with the elastomer not having become so distended that it has been made vulnerable to rupture.

Inelastic and therefore incapable of intrinsic stretching, the solid expandable structure expands diametrically and longitudinally by progressively and mechanically flattening in response to the increased average blood pressure associated with growth. Bursting, or rupturing, along a seam is precluded by molding the entire prosthesis as a unit and seamlessly joining it by inline coupling jackets through-and-through stitched to the native stump at either end. With a tensile strength of around 25,000 psi for polyetheretherketone, once the structure is fully extended, hence, completely flattened, the prosthesis should remain incapable of bursting or rupture, even in response to a direct blow. Moreover, unless the prosthesis is exceptionally short, a frontal impact is unlikely to apply so much tension on the stitches that the outer half-jackets would be pulled off from the substrate native stumps.

### Treatment of Deep Vascular Anomalies Refractory to Embolization or Excision

Vascular anomalies, truncular, or axial, and especially extratruncular, or involving small branches, such as anomalous arteriovenous anastomoses and venous malformities embedded in tissue, may occur at a greater depth within the body alone or with other vascular malformities in polyangiopathies. Those truncal are less often severely symptomatic and painful than those extratruncal (see, for example, Kim, T. H., Choi, J. W., and Jeong, W. S. 2023. “Current Concepts of Vascular Anomalies,” (Online), Archives of Craniofacial Surgery (Seoul, South Korea) 24(4): 145-158; Castillo-Rangel, C., Marin, G., Hernandez-Contreras, K. A., Zarate-Calderon, C., Vichi-Ramirez, M. M, and 11 others 2022. “Atlas of Nervous System Vascular Malformations: A Systematic Review,” Online, MDPI [Multidisciplinary Digital Publishing Institute] Life (Basel, Switzerland) 12(8): 1199; Zyck, S., Davidson, C, L., and Sampath, R. 2022. “Arteriovenous Malformations,” Online, Treasure Island, Florida: StatPearls Publishing Co; Ustaszewski, A., Janowska-Glowacka, J., Wolynska, K., Pierzak, A., and Badura-Stonka, M. 2020. “Genetic Syndromes with Vascular Malformations-Update on Molecular Background and Diagnostics,” Online, Archives of Medical Science (Poznan, Poland) 17(4): 965-991; Loose, D, A. and Mattassi, R, E. 2017. “Hamburg Classification: Vascular Malformation,” (Online), in Kim, Y.-W., Lee, B.-B., Yakes, W. F., and Do, Y.-S. (eds.), Congenital Vascular Malformations, Berlin and Heidelberg, Germany: Springer Verlag; Steiner, J, E. and Drolet, B, A. 2017. “Classification of Vascular Anomalies: An Update,” Online, Seminars in Interventional Radiology (New York, New York) 34(3): 225-232; Behravesh, S., Yakes, W., Gupta, N., Naidu, S., Chong, B. W., Kademhosseini, A., and Oklu, R. 2016. “Venous Malformations: Clinical Diagnosis and Treatment,” Online, Cardiovascular Diagnosis and Therapy (Sheung Wan, Hong Kong, China) 6(6): 557-569; Nosher, J, L., Murillo, P. G., Liszewski, M., Gendel, V., and Gribben, C, E. 2014. “Vascular Anomalies: A Pictorial Review of Nomenclature, Diagnosis, and Treatment,” Online, World Journal of Radiology 6(9): 677-692; Nozaki, T., Nosaka, S., Miyazaki, O., Makidono, A., Yamamoto, A., and 5 others 2013. “Syndromes Associated with Vascular Tumors and Malformations: A Pictorial Review,” Radiographies (Oak Brook, Illinois) 33(1): 175-195).

Where postoperative bleeding is a concern, tiny inline coupling jacket-secured expandable lines can be used along the leptomeninges (but not deep inside the brain) (see, for example, Quintin, S., Figg, J. W., Mehkri, Y., Hanna, C. O., Woolridge, M. G., and Lucke-Wold, B. 2023. “Arteriovenous Malformations: An Update on Models and Therapeutic Targets,”, Online, Journal of Neuroscience and Neurosurgery (London, England) 13(1): 250; Ajiboye, N., Chalouhi, N., Starke, R. M., Zanaty, M., and Bell, R. 2013. “Cerebral Arteriovenous Malformations: Evaluation and Management,” Scientific World Journal (New York, New York) 2014:649036). Otherwise, while alluded to by Zyck et al. 2022 and others, deep extracranial, deep somatic, and leptomeningeal, such as a Sturge-Weber malformations, at the periphery of the brain are those addressed.

The leptomeningeal application of inline coupling jacket-secured lines is considered only when medical management has failed, exceptionally, as when neurological symptoms do not present until adulthood or a rarer infantile weakness or malaise form of the disease is present in infancy, seizures that would warrant a hemispherectomy are not present, and access to the lesion is such that surgical removal through entry into the cranium is unavoidable (see, for example, Ryu, J. Y., Eo, P. S., Lee, J. S., Lee, J. W., Lee, S., and 5 others 2019. “Surgical Approach for Venous Malformation in the Head and Neck,” Online, Archives of Craniofacial Surgery (Seoul, South Korea) 20(5): 304-309; Comi, A. M. 2011. “Presentation, Diagnosis, Pathophysiology, and Treatment of the Neurologic Features of Sturge-Weber Syndrome,” Online, Neurologist 17(4): 179-184).

The application of inline coupling jacket-secured expandable lines along the leptomeninges, following the elimination of a symptomatic and/or painful Sturge-Weber vascular malformation, comprises two steps. The first is the use of a drug delivery isolated simple straight bloodline to repair an excised tangle, substantially dispelling the risk of postoperative bleeding or a recurrence of the lesion (Alakbarzade, V. and Pereira, A, C. 2018. “Cerebral Catheter Angiography and Its Complications,” Practical Neurology 18(5): 393-398).

The second step depends upon whether the site of the malformity warrants the direct pipe-targeting of medication into the entry inline coupling jacket securing the bloodline or bloodlines used to effect the repair. If so, this is accomplished by tunneling the drugline or druglines from the drug reservoir or reservoirs implanted subcutaneously in the pectoral region subcutaneously up through the neck, into the head, and the drugline or druglines inserted in the accessory channel or channels of the entry inline coupling jacket. If the malformation is not obtrusively large and does not cause severe symptoms or pain, consideration may be given to applying a tiny side-entry jacket to an accessible vessel for the direct delivery thereinto of an anti-inflammatory.

Readily observed with an ultrasound handpiece, rather than run more deeply, bloodlines and druglines are best tunneled between the integument and the epimysium fascia where the possibility of strangulation is avoided. The same route is followed to effect communication with the deeper central nervous system than the leptomeninges: to gain access to the subarachnoid space and cerebrospinal fluid, hence, the entire central nervous system, requires an ordinary drugline from the drug reservoir under the control of the implanted disorder response system connected to a conventional Ommaya reservoir.

Channeling of the catheteric drugline from the drug reservoir implanted in the pectoral region to the Ommaya reservoir is through the neck as is that for connection to a prosthetic bloodline used to repair a vascular malformation, with connection to the Ommaya reservoir the same as that of the catheter associated with the reservoir (see, for example, Zubair, A. and De Jesus, O. 2023. “Ommaya Reservoir,” Online, Treasure Island, Florida: StatPearls Publishing Co.; Magill, S. T., Choy, W., Nguyen, M. P., and McDermott, M. W. 2020. “Ommaya Reservoir Insertion: A Technical Note” Online, Cureus (Palo Alto, California) 12(4): e7731). An Ommaya reservoir is placed when repetitive access to the cerebrospinal fluid will be needed and the alternative would be multiple lumbar access, or spinal taps.

The two-way in-out therapeutic-diagnostic injection-aspiration function of the Ommaya reservoir is automated in that not only can diagnose samples of cerebrospinal fluid be scheduled for evaluation by implanted sensors-some enumerated above in section 2a, Background of the Invention. General—but chemotherapeutics, antibiotics, antivirals, anti-inflammatoires, can be programmed to be infused in response thereto. Whether the system can be fully implanted depends upon the number and volume of the drug reservoirs needed; if the full complement cannot be accommodated intracorporeally, a body pack is used.

A need for repetitive access to the cerebrospinal fluid responds to numerous diseases of or involving the central nervous system (see, for example, Batool, A. and Kasi, A. 2023. “Leptomeningeal Carcinomatosis,” Online, Treasure Island, Florida: StatPearls Publishing Co.; Cantu, R. M. and Das, Joe M. 2023. “Viral Meningitis,” Online, Treasure Island, Florida: StatPearls Publishing Co.; Dyckhoff-Shen, S., Bewersdorf, J. P., Teske, N. C., Volk, S., Pfister, H. W., Koedel, U., and Klein, M. 2023. “Characterization and Diagnosis Spectrum of Patients with Cerebrospinal Fluid Pleocytosis,” Online, Infection [in press September 1, 23]; Hersi, K., Gonzalez, F. J., and Kondamudi, N. P. 2023. “Meningitis,” Online, Treasure Island, Florida: StatPearls Publishing Co.; Runde, T. J., Anjum, F. and Hafner J. W. 2023. “Bacterial Meningitis,” Online, Treasure Island, Florida: StatPearls Publishing Co.; Wang, S., Yang, W., Zhu, M., Wang, X., Pan, L, and 5 others 2023. “Cerebrospinal Fluid Protein Levels are Elevated 100 Times in a Leptomeningeal Metastasis Patient: A Case Report and Literature Review,” Online, Frontiers in Neuroscience (Lausanne, Switzerland) 17:1174309; Shahan, B., Choi, E. Y/, and Nieves G. 2021. “Cerebrospinal Fluid Analysis,” Online, American Family Physician (Leawood, Kansas) 103(7): 422-428, erratum 103(12): 713; Benameur, K., Agarwal, A., Auld, S. C., Butters, M. P., Webster, A. S., and 7 others 2020. “Encephalopathy and Encephalitis Associated with Cerebrospinal Fluid Cytokine Alterations and Coronavirus Disease, Atlanta, Georgia, USA, 2020,” Online, Emerging Infectious Disease (Atlanta, Georgia) 26(9): 2016-2021; Zheng, G., Zhang, Y., Zhang, L., Qian, L., Cai, Y., and 8 others 2020. “Evaluation of a Micro/Nanofluidic Chip Platform for Diagnosis of Central Nervous System Infections: A Multi-center Prospective Study,” Online, Nature Research Scientific Reports (London, England) 10(1): 1538; Hrishi, A. P. and Sethuraman, M. 2019. “Cerebrospinal Fluid (CSF) Analysis and Interpretation in Neurocritical Conditions,” Online, Indian Journal of Critical Care Medicine (New Delhi, India) 23 (Supplement 2): S115-S119; Wilson, R., Osborne, C., Halsey, C. 2018. “The Use of Ommaya Reservoirs to Deliver Central Nervous System-directed Chemotherapy in Childhood Acute Lymphoblastic Leukaemia,” Online, Pediatric Drugs 20(4): 293-301; Seehusen, D. A., Reeves, M. M., and Fomin, D, A. 2003. ““Cerebrospinal Fluid Analysis,” Online, American Family Physician (Leawood, Kansas) 68(6): 1103-1108).

When a leptomeningeal vascular malformation is painful and/or symptomatic but excision is not an option (because a large tangle of tiny vessels makes discerning the exact flow of blood undependable), these malformations can often be alleviated through embolization, although even when successful, healing usually takes weeks after the procedure during which the patient is prescribed an oral, seldom fully effective, analgesic. Embolization in lieu of complete excision cannot be predicted to prevent a reformation, or recrudescence, of the malformity.

Deeper inside the body, access to a vascular defect from outside the body-if intrahepatic, for example—will prevent taking advantage of embolization as a means of avoiding surgery. That is, embolization or excision may be used, but access to the site will have necessitated dissection. When surgery is unavoidable and the consequences of embolization or excision are unclear and could interfere with essential high blood flow, bypass expandable structure lines can be considered.

These operations are microsurgical, often complex, and call for an interdisciplinary team of experienced specialists (Chung, T. C., Thang, L. M., Giang, N, L., Trung, N. A., Qui, L. V., and 5 others 2022. “Recurrent Head and Neck Arteriovenous Malformations: A Case Report,” Online, Radiology Case Reports (Amsterdam North Holland) 18(3): 766-770; Walter, A., Calite, E, Müller, A., Kalff, J. C., Meyer, C., and 3 others 2022. “Prenatal Diagnosis and Management of a Giant Intrahepatic Arteriovenous Malformation-Sonographic Findings, Clinical Implications, and Treatment,” Online, Clinical Case Reports(Chichester, England) 10(8): e6175; Eivazi, B. and Werner, J, A. 2014. “Extracranial Vascular Malformations (Hemangiomas and Vascular Malformations) in Children and Adolescents-Diagnosis, Clinic, and Therapy,” Online, German Medical Science Current Topics in Otorhinolaryngology, Head and Neck Surgery (Bonn, Germany) 13: Doc02).

When microsurgery is unavoidable, will be intricate, and prone to complications, an alternative remedy that bypasses—that is, temporarily or permanently bridges around the defect to allow its treatment or excision and replace it with one or more prosthetic lines warrants consideration. While very small, the same circumferentially alternating flats and vaults expansion structure can be applied to the production of vascular prostheses as simple bloodlines to replace anomalous vessels in prenates, neonate, infants, and toddlers.

Discovered in an infant or toddler in proportion to its symptoms and pain, avoiding the need for later revision makes it imperative that any synthetic bloodlines or druglines will expand to accommodate growth and do so without the risk of disconnection, bleeding, or strangulation of intervening anatomy. The implanted control center not usually expected to warrant replacement later in life, to support one or a few such prostheses with directly pipe-targeted symptom-responsive medication requires a response system with drug reservoirs which can provided in a size small enough to place in an infant.

Where excision and anastomosis arouse concerns for a lack of post healing anastomotic strength and the possibility of dehiscence, a tiny inline coupling jacket-secured line would prove more dependable. The propensity for dehiscence and the need to accommodate growth without reentry foremost in treating an infant, the line used is of the kind specified above. Repair using prosthetic components is not recommended where conventional endovascular microsurgery can effectuate a durable repair.

In such tight quarters, side-entry valves and servovalves will be too large. Instead of controlling the release of drugs into the connectors and lines, the control of drug release is at the outlet of the peristaltic pump of each drug reservoir rather than local to the site of treatment. These components include tiny passive side-entry and inline coupling jackets to connect shunts, bypasses, and bridging, and tie-lines of types delineated in section 2b(2)(c)(c3), entitled Types of Inline Coupling jacket-secured Bridging, or Tie-lines, as well as components described in the copending applications specified above in the opening section entitled Cross Reference to Related Applications. The control of an isolated repair demands little in the way of implanted components.

### Recent Findings Adaptable for Administration by a Fully Implanted Automatic Disorder Response System

With respect to both the applications referred to herein and the practice of general medicine, the druglines controlled by an implanted microprocessor in a prosthetic disorder response system can be used to directly pipe-target stem cells such as contained within bone marrow, platelet rich plasma, and any other regenerative substance automatically on a scheduled basis coordinated with sensor feedback as to the immediate condition. The automatic scheduled release of drugs into the bloodstream by a fully implanted disorder response system overcomes numerous impediments such as patient noncompliance due to extremes of age as well as normal adults preoccupied with other matters.

“Scientists have designed special immune cells that protect transplanted pancreatic cells from attack in mice . . . scientists have designed immune cells that protect stem cell transplants from being rejected by the body—and they could someday open the door for a cure for diabetes,” (Ghose, T. 2024. “Scientists Make 1-of-a-Kind Immune Cells to Guard Transplants from Attack,” Online, Live Science (New York, New York) livescience.com/health/medicine-drugs/; Reddy, N. R., Maachi, H., Xiao, Y., Simic, M. S., Yu, W., and 9 others 2024. “Engineering Synthetic Suppressor T Cells that Execute Locally Targeted Immunoprotective Programs,” Science (Washington, D.C.) 386(6726): eadl4793). A caveat is that results obtained with murid subjects often fail to translate to humans.

Researcher Ashley “Makela and a team of multidisciplinary and multi-institutional members used a drug that signals the body to boost or inhibit a particular reaction, called a metabolic modulator. This drug was incorporated into an amorphous polylactide—a biomaterial used to make medical implants and the material was implanted in mice . . . the researchers imaged different kinds of immune cells around the implant site for up to 10 weeks . . . “Our findings have significant implications for improving patient recovery times, reducing postsurgical complications like chronic inflammation and implant rejection and potentially saving costs,” said Makela . . . and they may eventually affect the way medical device manufacturers and pharmaceutical scientists approach medical implants,” (Perideaux, M. 2024. “Researchers Reprogram Immune Cells to Decrease Rejection of Medical Implants,” Online, São Paulo, Brazil: Medical Xpress; Maduka, C. V., Schmitter-Sanchez, A. D., Makela, A. V., Ural, E., Stivers, K. B., and 17 others 2024. “Immunometabolic Cues Recompose and Reprogram the Microenvironment around Implanted Biomaterials,” Nature Biomedical Engineering (London, England) 8(10): 1308-1321).

Recently discovered have been “clonogenic endothelial-macrophage (EndoMac) progenitor cells in the adventitia of the embryonic and postnatal mouse aorta which are independent of Flt3-mediated bone marrow hematopoiesis and derive from an early embryonic CX₃CR1⁺ and CSF1R⁺ source. In evaluating research results based upon other species, it is important to remember that the results obtained with may not translate to humans, especially with results obtained from murids. [Paraphrasing] applying the principles of precision engineering of biological systems to the design of artificial virus-like particles capable of encapsulating genes can be used to deliver genetic material into cells. “Specifically, the virions are formed from (l [levorotatory]-amino acids) assembling alternately with their mirror reflections, known as d [dextrorotatory]-amino acids, which assemble to create virus-like shells which can be designed to enclose nucleic acids of different sizes,” (Science X Staff/National Physical Laboratory 2024. “Precision Engineering Enables Design of Virus-like Particles that Can Deliver Genetic Material into Cells,” Online, São Paulo, Brazil: Medical Xpress; Noble J E, Hsiao Y W, Kepiro I E, De Santis E, Hoose A, and 7 others 2024. “A Nonlinear Peptide Topology for Synthetic Virions,” ACS [American Chemical Society] Nano (Washington, D.C.) 18(43): 29956-29967).

It is plausible that the directly pipe-targeted and contained release of therapeutic substances will minimize if not completely eliminate the misdelivery of agents that cause complications. These bipotent progenitors are proliferative and vasculogenic, contributing to adventitial neovascularization and formation of perfused blood vessels after transfer into ischemic tissue,” (Williamson, A. E., Liyanage, S., Hassanshahi, M., Dona, M. S. I., and Toledo-Flores, D., and 22 others 2024. “Discovery of an Embryonically Derived Bipotent Population of Endothelial-macrophage Progenitor Cells in Postnatal Aorta,” Online, Nature Communications (London, England) 15(1): 7097). Cahill, T. J., Sun, X., Ravaud, C., Villa Del Campo, C., Klaourakis, K., and 11 others 2021. “Tissue-resident Macrophages Regulate Lymphatic Vessel Growth and Patterning in the Developing Heart,” Online, Development (Cambridge, England) 148(3): dev 194563)”

Another recent discovery adaptable for directly pipe-targeted automatic administration by a fully implanted prosthetic disorder response system is that impairment in a link between two key pathways—specifically, the relationship between interleukin ILC3s and interleukin 23—which regulates the immune system in mammals is responsible for conditions such as chronic inflammation in the gut. The treatment of skip lesions along the gut is briefly addressed in U.S. Pat. No. 11,389,171, entitled Integrated System for the Infixion and Retrieval of Implants, and the use of ductus side-entry jackets in support of a fully implanted automatic disorder response system as described in U.S. Pat. No. 11,759,186, entitled Ductus Side-entry Jackets and Prosthetic Disorder Response Systems, materially augments the level of treatment possible.

Yet another recent discovery adaptable for directly pipe-targeted automatic administration by a fully implanted prosthetic disorder response system is that impairment in a link between two key pathways—specifically, the relationship between interleukin ILC3s and interleukin 23—which regulates the immune system in mammals is responsible for conditions such as chronic inflammation in the gut. The treatment of skip lesions along the gut is briefly addressed in U.S. Pat. No. 11,389,171, entitled Integrated System for the Infixion and Retrieval of Implants, and the use of ductus side-entry jackets in support of a fully implanted automatic disorder response system as described in U.S. Pat. No. 11,759,186, entitled Ductus Side-entry Jackets and Prosthetic Disorder Response Systems, materially augments the level of treatment possible.

As shown for a kidney in FIGS. 6, 13A and 13B of U.S. Pat. No. 11,013,858, using a fully implanted automatic disorder response system, remedial agents can be scheduled for direct automatic targeting at or into emergent or premetastatic malignant prostate tumors. “Like other tumors, prostate cancer cells need a lot of nutrients to support their rapid growth. As nutrients are depleted, a protein called GCN2 signals the cells to make more fuel for growth. The team reasoned that a drug that shuts GCN2 down would render the cancer unable to make enough fuel to survive.”

However, “inhibiting GCN2 did slow the growth of the tumor cells, but it didn't kill them. That's when we discovered the cancer has a backup plan., The team went on to show that a protein called p53 was cancer's “Plan B,” The p53, which is functionally retained in most prostate cancers-unlike in other forms of cancer-signals to restrict cell division and gather nutrients. Prostate cancers could be effectively destroyed when the researchers inhibited both GCN2 and p53,” Science X Staff 2024. “Researchers Find New Way to ‘Starve’ Prostate Cancer Tumors at the Cellular Level,” Online, Isle of Man, England: Medical Xpress; Cordova, R. A., Sommers, N. R., Law, A. S., Klunk, A. J., Brady, K. E., and and 6 others 2024. “Coordination between the elF2 Kinase GCN2 and p53 Signaling Supports Purine Metabolism and the Progression of Prostate Cancer,” Science Signaling (Washington, D.C.) 17(864): eadp1375).

“When cells undergo damage or dysfunction, they often enter a state called senescence. This process halts their proliferation and triggers a proinflammatory response known as the senescence-associated secretory phenotype (SASP). SASP facilitates immune system activity by recruiting immune cells to remove these senescent cells, thus preserving tissue integrity . . . tumors, can harbor senescent cells both before and after treatment, these cells, while not proliferating, impact the tumor microenvironment through senescence-associated secretory phenotype.”

“Depending on the cancer's characteristics and the treatment applied, senescence-associated secretory phenotype can have varying effects, ranging from promoting antitumor Immune responses to creating barriers that protect tumors. Immune clearance of senescent cells primarily involves innate immune components like macrophages and natural killer cells. These cells target senescent cells for elimination. Adaptive immunity also plays a role, as seen in studies where senescent hepatocytes expressing oncogenic mutations activate CD4 T cells, which in turn recruit macrophages to remove premalignant cells. However, senescent cells also upregulate inhibitory signals like HLA-E, shielding them from immune attack. This dual role underscores the complexity of senescence in cancer biology. Groundbreaking research by scientists at Institute for Research in Biomedicine Barcelona has explored how inducing senescence in cancer cells can enhance immune system efficacy. Their study, published in Cancer Discovery, reveals promising implications for cancer treatment. (Shavit, J. 2024 “Groundbreaking Vaccine Successfully Treats Skin and Pancreatic Cancer,” Brighterside of News: Los Angeles, California; Marin, I., Boix, O., Garcia-Garijo, A., Sirois, I., Caballe, A., and 13 others 2023. “Cellular Senescence is Immunogenic and Promotes Antitumor Immunity,” Online, Cancer Discovery (Philadelphia, Pennsylvania) 13(2): 410-431; Prieto, L. I., Sturmlechner, I., Goronzy, J. J., and Baker, D. J. 2023. “Senescent Cells as Thermostats of Antitumor Immunity,” Online, Science Translational Medicine (Washington, D.C.) 15(699): eadg 7291).

Another recent bacteria-directed discovery adaptable for directly pipe-targeted automatic administration by a fully implanted prosthetic disorder response system is the delivery of functional microporous materials known to regulate anticancer activity of complex bacterial systems.

Used are antitumor anticancer bacterial strains A-gyo and UN-gyo, bacteria having been prepared along with a simple method for growing AUN [A-gyo (Proteus mirabilis) and UN-gyo (Rhodopseudomonas palustris) combined intratumoral bacterial consortium] using a light-prepared scaffold (Miyahara, M., Doi. Y., Takaya, N., and Miyako, E. 2024. “Photocatalytic Scaffolds Enhance Anticancer Performances of Bacterial Consortium AUN,” Online, Chemical Engineering Journal (Lausanne, Switzerland) 499.156378; Japan Advanced Institute of Science and Technology 2024. “New Bacteria-based Therapy Shows Promise for Fighting Cancer,” ScienceDaily, Online at www.sciencedaily.com/releases/2024/10/241015141621.htm).

“Blocking CTLA-4 on ILC3s may lead to severe gut inflammation which can cause patients to discontinue their cancer treatment . . . [These findings] also provides clues on how to harness this pathway to fight cancer and alleviate a serious side effect of cancer immunotherapy” (Ahmed, A., Joseph, A. M., Zhou, J., Horn, V., Uddin, J., and 8 others 2024. “CTLA-4-expressing ILC3s Restrain Interleukin-23-mediated Inflammation,” Nature (Basingstoke, England) 630(8018): 976-983; Science X Staff 2024. “Putting the Brakes on Chronic Inflammation: Study Discovers Link between Two Key Pathways,” Online, São Paulo, Brazil: Medical Xpress).

“ . . . the structure of how an inhibitory molecule, LAG3 [lymphocyte activation gene 3], interacts with its main ligand and provides a new targeted approach to improving the effectiveness of immunotherapy for certain forms of cancer” has been published in Science Immunology. “The publication is the first to show the crystal structure of a human LAG-3/HLA-II complex and provides a better foundation for development of blocking LAG-3 therapeutics,” (Science X Staff/Monash University 2024. “Molecular Insights Unlock a Targeted Approach to Cancer Immunotherapy,” Online, São Paulo, Brazil: Medical Xpress; Petersen, J., Llerena, C, Golzarroshan, B., Faoro, C., Triebel, F., and Rossjohn, J. 2024. “Crystal Structure of the Human LAG [lymphocyte activation gene]-3-HLA [human leucocyte antigen]-DR1 [D-retro-inverso]—Peptide Complex,” Science Immunology (Washington, D.C.) 9(102): eads5122).

“This work was conceived as a comparative study of the mechanisms and dynamics of resistance acquisition for two drugs and two bugs,” said senior author Andrei L. Osterman, Ph.D., at Sanford Burnham Prebys. “Comparing mutational landscapes triggered by the same drug in two distinct bugs allows us to deduce both shared and unique evolutionary trajectories toward resistance. A comparison of two drugs in the same bug reveals shared and unique mechanistic features of fundamental and translational importance, from drug discovery to rational optimization of treatment regimens . . . both pathogens studied are gram-negative bacteria with shared characteristics but also notable differences,” (Science X Staff 2024. “Novel Mixture of mRNA in Nanoparticles Show Therapeutic Potential against Tumor Progression” Online, Phys. Org (Isle of Man, England); Leyn, S. A., Kent. J. E., Zlamal, J. E., Elane, M. L. Vercruysse, M. and Osterman, A. L. 2023. “Two Classes of DNA Gyrase Inhibitors Elicit Distinct Evolutionary Trajectories toward Resistance in Gram-negative Pathogens,” bioRxiv [in press]. September 21:2023.06.26.546596).

Gene inhibition appears to provide a way to overcome the resistance to cisplatin of cancer cells. This finding could improve the effectiveness of the widely used platinum-based medication and reduce the required dosage, minimizing side effects,” (Cedars-Sinai Medical Center/Science X staff 2024. “Gene Inhibition Provides Potential Way to Overcome Cancer Cells' Resistance to Chemotherapy Drug Cisplatin,” Online, São Paulo, Brazil: Medical Xpress; Feldman, L, E. R., Mohapatra, S., Jones, R. T., Scholtes, M., Tilton, C. B., and 13 others 2024. “Regulation of Volume-regulated Anion Channels Alters Sensitivity to Platinum Chemotherapy,” Science Advances (Washington, D.C.) 10(50): eadr9364).

Recently identified is “ . . . a molecule that can help the intestines to heal after damage while suppressing tumor growth in colorectal cancer,” (Karolinska Institutet Staff 2024. “Scientists Identify Molecule that Promotes Gut Regeneration and Limits Tumor Growth,” Online at https://medicalxpress.com/news/2024-11-scientists-molecule-gut-regeneration-limits.html; Das S. Parigi S M, Luo X, Fransson J. Kern B C, and 26 others 2024. “Liver X Receptor Unlinks Intestinal Regeneration and Tumorigenesis,” Online, Nature (Basingstoke, England) [in press] November 20). The objective is to replace immunosuppression with tissue regeneration in inflammatory diseases of the gut such as inflammatory bowel disease (IBD) without inducing malignancy. “This new therapeutic molecule has the potential to treat not only IBD patients but also cancer patients to prevent chronic bowel disorders after radiotherapy and/or chemotherapy.”

Yet another discovery adaptable for directly pipe-targeted automatic administration by a fully implanted prosthetic disorder response system are three cancer and demyelination counteractive substances comprising “two synthetic thiocarbamates and one plant-derived flavonoid, sulfuretin, that effectively blocked cell migration-inducing and hyaluronan-binding protein (CEMIP) activity in live cells, including a tumorigenic cell line and primary cultures of oligodendrocyte progenitor cells (OPC),” (Peters, A., Banine, F., Yasuhara. K., Shock, C. C., Back, S. A., and 7 others 2024. “Distinct Chemical Structures Inhibit the CEMIP [cell migration inducing and hyaluronan inducing binding protein] Hyaluronidase and Promote Oligodendrocyte Progenitor Cell Maturation,” Online, Journal of Biological Chemistry (New York, New York) in press). The applicable substances are extracted from flowers (Robinson, E. 2024. “Natural Compound Found in Flowers Blocks Activity of an Enzyme Involved in Multiple Sclerosis and Cancer,” Online, Phys. Org (Isle of Man, England) October 2024).

“The discovery of “new molecular mechanisms underlying DNA repair dysregulation in prostate cancer cells” may lead to “the development of new targeted therapies for patients that have become resistant to standard treatment” (Rohman, M. 2024. “Targeting Novel Molecular Mechanisms May Repair Damaged DNA in Cancer Cells,” Online, Sao Paulo, Brazil: Medical Xpress; Meng, Q., Shen, J., Ren. Y., Liu, Q., Wang, R., and 18 others 2024. “EZH2 [enhancer of zeste homolog 2] Directly Methylates PARP1 [poly(adenosine diphosphate-ribose) polymerase-1] and Regulates Its Activity in Cancer,” Online, Science Advances (Washington, D.C.) 10(48): ead12804).

“Pretreatment with EZH2 inhibitor leads to higher recruitment of CAR-T cells (red) into the lymph node tumor compared with control. Pretreatment with EZH2 inhibitor also leads to closer contacts between lymphoma B cells and T cells in culture., The inhibitors target the EZH2 enzyme, whose activity in tumor cells is now recognized as a significant factor in many cancers,” Science X Staff 2024. “Anticancer Drugs Could Make Immunotherapies More Effective,” Online, São Paulo, Brazil: Medical Xpress; Isshiki, Y., Chen, X., Teater, M., Karagiannidis, I., Nam, H., and 20 others 2024. “EZH2 Inhibition Enhances T Cell Immunotherapies by Inducing Lymphoma Immunogenicity and Improving T Cell Function,” Cancer Cell (Cambridge, Massachusetts) November 30: S1535-6108(24) 00441-0).

“The delivery of nanomedicines using gas bubbles has shown itself to be a unique way of transporting cytotoxins to the lungs of cancer patients . . . enabling precise and focused treatments, and the local action of the drugs also prevents a range of side-effects,” (SINTEF [Stiftelsen for industriell og teknisk forskning/Foundation for Industrial and Technical Research in Trondheim, Norway] 2024. Using Gas Bubbles to Precisely Deliver Nanomedicines Shows Promise for Lung Cancer Treatment,” Online, Phys. Org (Isle of Man, England); Snipstad, S., Sulheim, E., Aslund, A. K. O., Hyldbakk, A., Wagbo, A. M., Klinkenberg, G., and Morch, Y. 2024. “Nanoparticle-loaded Microbubbles for Treatment of Lung Cancer,” Online, European Journal of Pharmaceutical Sciences (Amsterdam, North Holland, Netherlands) 199:106804).

“EZH2, an enzyme crucial for regulating gene expression, often becomes overactive in lymphoma cells due to genetic mutations. This hyperactivity suppresses the immune system's ability to recognize and attack tumors. Inhibiting EZH2 can reverse this immunosuppression, making tumor cells more vulnerable to immune attacks” (Shavit, J. 2024. “New Anticancer Drugs Could Significantly Improve Immunotherapy,” Online at www.msn.com/en-us/science/biochemistry/new-anticancer-drugs; Beguelin, W., Popovic, R., Teater, M., Jiang, Y., Bunting, K, L., and 28 others 2013 “EZH2 [enhancer of zeste homolog 2] is Required for Germinal Center Formation and Somatic EZH2 Mutations Promote Lymphoid Transformation,” Online, Cancer Cell (Cambridge, Massachusetts) 23(5): 677-692).

“ . . . blocking growth differentiation factor 15 (GDF-15) [program cell death protein/growth/differentiation factor 15, a protein encoded by the GDF15 gene] can counteract resistance to anti-PD-1 [CD279 cluster of differentiation 279)] and anti-PD-L1 [programmed death-ligand 1 (PD-L1)/cluster of differentiation 274 (CD274)/B7 homolog 1 (B7-H1), a protein encoded by the CD274 gene] therapies in solid tumors.” (Jackson, J. 2024. “GDF-15 Inhibitors Show Promise against Hard-to-treat Tumors in Clinical Trial,” Online, Sao Paulo, Brazil: Medical Xpress; Melero, I., de Miguel Luken, M., de Velasco, G., Garralda, E., Martin-Liberal, J., and 67 others 2024. “Neutralizing GDF-15 Can Overcome Anti-PD-1 and Anti-PD-L1 Resistance in Solid Tumours,” Online, Nature (London, England) [in press]).

Whether a fully implanted disorder response system with vascular valve and/or servovalve end effectors can be adapted to reduce if not eliminate the complications of administering gene editing through CRISPER-Cas 9 and/or CRISPER-Cas 10 during the post embryonic stage would appear feasible but calls for detailed research (“Ideal delivery vectors should have the advantages of non-toxicity, well targeting property, high efficiency, low cost, and biodegradability. At present, three main delivery methods have been employed in delivering CRISPR-Cas components, including physical, viral and non-viral methods,” see, for example, Xu, Y., and Li, Z. 2020. “CRISPR-Cas Systems: Overview, Innovations and Applications in Human Disease Research and Gene Therapy,” Computational and Structural Biotechnology Journal (Amsterdam, North Holland, Netherlands) 18:2401-2415).

Another avenue for research into the potential therapeutic applications of a fully implanted automatic disorder response system using vascular valves as end-effectors pertains to synthetic cell type-specific cis-regulatory elements, or cis-regulatory modules, which control gene expression (Gosai, S J., Castro, R. I., Fuentes, N., Butts, J. C., Mouri, K., and 9 others 2024. “Machine-guided Design of Synthetic Cell Type-specific cis-regulatory Elements,” Online, Nature (Basingstoke, England) 634(8036): 1211-1220; Xu, L. and Liu, Y. 2024. “Identification, Design, and Application of Noncoding Cis-regulatory Elements,” Online, MDPI [Multidisciplinary Digital Publishing Institute] Biomolecules 14 (Basel, Switzerland) (8): 945; Liu, X, Chen, M., Qu, X., Liu, W., Dou, Y., and 4 others 2023. “Cis-regulatory Elements in Mammals,” Online, MDPI [Multidisciplinary Digital Publishing Institute] International Journal of Molecular Sciences (Basel, Switzerland) 25(1): 343).

A related subject pertains to cell-type-directed design of synthetic enhancers (see, for example, Bohrer, C. H., Fursova, N. A., and Larson, D, R. 2024. “Enhancers: A Focus on Synthetic Biology and Correlated Gene Expression,” ACS [American Chemical Society] Synthetic Biology (Washington, D.C.) 13(10): 3093-3108; Pfenning, A, R. 2024. “AI-designed DNA Sequences Regulate Cell-type-specific Gene Expression,” Nature (Basingstoke, England) 634(8036): 1059-1061; Taskiran, I I., Spanier, K. I., Dickmanken, H., Kempynck, N., Pancikova, A., and 8 others 2024. “Cell-type- directed Design of Synthetic Enhancers,” Online, Nature (Basingstoke, England) 626(7997): 212-220).

Yet another area adaptable to allow administration by a fully implanted automatic disorder response system where the inherent toxicity of the agent if not physically pipe-targeted is a source of complications as might otherwise disallow the use thereof are “ . . . chimeric antigen receptor (CAR) T cell T lymphocytes which have been genetically engineered to express a synthetic receptor that recognizes a tumor cell surface antigen and causes the T cell to kill the tumor cell,” (see, for example, Bourlon, C., Roddie, C., Menne, T., Norman, J., O'Reilly, M., and 18 others 2024. “Outcomes after Chimeric Antigen Receptor T-Cell Therapy across Large B-cell Lymphoma Subtypes,” Online, Haematologica (Pavia, Italy) 109(8): 2716-2720; Brudno, J. N., Maus, M. V., and Hinrichs, C S. 2024. “CAR T Cells and T-cell Therapies for Cancer: A Translational Science Review,” Journal of the American Medical Association (Chicago, Illinois) [in press] November 4; Bui, T. A., Mei, H., Sang, R., Ortega, D. G., and Deng, W. 2024. “Advancements and Challenges in Developing in Vivo CAR T Cell Therapies for Cancer Treatment,” Online, EBioMedicine (Amsterdam, North Holland, Netherlands) 106:105266; Ercilla-Rodriguez, P., Sanchez-Diez, M., Alegria-Aravena, N., Quiroz-Troncoso, J., Gavira-O'Neill, C. E., Gonzalez-Martos, R., and Ramirez-Castillejo, C. 2024. “CAR-T Lymphocyte-based Cell Therapies; Mechanistic Substantiation, Applications, and Biosafety Enhancement with Suicide Genes: New Opportunities to Melt Side Effects, Online, Frontiers in Immunology (Lausanne, Switzerland) 15:1333150; Jacobson, C. A., Munoz, J., Sun, F., Kanters, S., Limbrick-Oldfield, E. H., and 8 others 2024. “Real-world Outcomes with Chimeric Antigen Receptor T Cell Therapies in Large B Cell Lymphoma: A Systematic Review and Meta-Analysis,” Online, Transplantation and Cellular Therapy (New York, New York) 30(1): 77.e1-77.e15; Johnson, P. C., Neckermann, I., Sadrzadeh, H., Newcomb, R., El-Jawahri, A. R., and Frigault, M. J. 2024. “Clinical Outcomes and Toxicity in Older Adults Receiving Chimeric Antigen Receptor T Cell Therapy,” Transplantation and Cellular Therapy (New York, New York) 30(5): 490-499; Li, J., Chen, H., Xu, C., Hu, M., Li, J., and Chang, W. 2024. “Systemic Toxicity of CAR-T Therapy and Potential Monitoring Indicators for Toxicity Prevention,” Online, Frontiers in Immunology (Lausanne, Switzerland) 15:1422591; Russo, E., Gambella, M., Raiola, A. M., Beltrametti, E., Zanetti, V., and 4 others 2024. “Acute Kidney Injury in Hematological Patients Treated with CAR-T Cells: Risk Factors, Clinical Presentation and Impact on Outcomes,” Online, Scientific Reports (London, England) 14(1): 26886; Shaikh, S. and Shaikh, H. 2024. “CART Cell Therapy Toxicity,” Online, Treasure Island, Florida: StatPearls Publishing Co.; Schroeder, T., Martens, T., Fransecky, L., Valerius, T., Schub, N., and 3 others 2024. “Management of Chimeric Antigen Receptor T (CAR-T) Cell-associated Toxicities,” Online, Intensive Care Medicine (New York, New York) 50(9): 1459-1469; Gazeau, N., Liang, E. C., Wu, Q. V., Voutsinas, J. M., Barba, P, and 19 others 2023. “Anakinra for Refractory Cytokine Release Syndrome or Immune Effector Cell- associated Neurotoxicity Syndrome after Chimeric Antigen Receptor T Cell Therapy,” Online, Transplantation and Cellular Therapy (New York, New York) 29(7): 430-437; Yi, D., Dong, N., Rubio Lopes-Garcia, L., Viñal, D., Bachmeier, C., Shah, B. D., and 8 others 2023. “Outcomes of CD19 [cluster of differentiation molecule; B-lymphocyte antigen]—directed Chimeric Antigen Receptor T Cell Therapy for Transformed Nonfollicular Lymphoma,” Online, Transplantation and Cellular Therapy (New York, New York) 29(6): 349.e1-349.e8; Gergis, M., Hsu, J., Yang, Y., Bi, X., Aljurf, M., and Gergis, U. 2022. Next-generation Chimeric Antigen Receptor T-cells,” Hematology/Oncology and Stem Cell Therapy (Riyadh, Saudi Arabia) 15(3): 117-121; Hu, Y., Zheng. W., Qiao. J., Cui, Q., Zhu, Y., Chang, A. H., and Huang, H. 2020. “Bullous and Exanthematous Lesions Associated with Chimeric Antigen Receptor T-Cell Therapy in a Patient with Diffuse Large B-cell Lymphoma,” Journal of the American Medical Association Dermatology (Chicago, Illinois) 156(9): 1026-1028).

Also pertaining to overcoming the challenges of applying CAR T-cell therapy to solid tumors as well as blood-borne or hematologic malignancies. “The cells on tumors are much more heterogeneous than those associated with lymphomas or leukemias, and solid tumor cells exist in a hostile, immunosuppressive microenvironment that can shut down any kind of anti-tumor response. Combination strategies, such as CAR T-cell therapy plus immune checkpoint inhibition” appear to hold the prospect of success. The report is limited in terms of specifics but the proponents are identified and can be contacted (Bankhead, C. 2024. “Overcoming the Challenges of Applying CAR T-Cell Therapy to Solid Tumors,” Online, New York, New York: Medpage Today).

And also related to the use of CAR-T cells relative to solid tumors is the ability to provide these with specialized receptors to “ . . . localize the activity of CAR T cells to solid tumors where soluble disease-associated factors are expressed, bypassing the major hurdle of on-target off-tumor toxicity in bystander organs,” (Piraner, D. I., Abedi, M. H., Duran Gonzalez., M J., Chazin-Gray, A., Lin, A., and 10 others 2024. “Engineered Receptors for Soluble Cellular Communication and Disease Sensing,” Nature (Basingstoke, England) [in press] November 14; see also, Haroun, G. and Gordon, E. M. 2024. “DeltaRex-G. Tumor Targeted Retrovector Encoding a CCNG1 [cyclin-G1 protein encoded in humans by the CCNG1 gene] Inhibitor, for CAR-T Cell Therapy-induced Cytokine Release Syndrome,” Online, Frontiers in Molecular Medicine (Lausanne, Switzerland) 4:1461151; Teng. F., Cui. T., Zhou, L., Gao, Q., Zhou, Q., and Li, W. 2024. “Programmable Synthetic Receptors: The Next Generation of Cell and Gene Therapies,” Online, Signal Transduction and Targeted Therapy (London, England) 9(1): 7; Zhu, I., Liu, R., Garcia, J. M., Hyrenius-Wittsten, A., Piraner, D. I., and 5 others 2022. “Modular Design of Synthetic Receptors for Programmed Gene Regulation in Cell Therapies,” Online, Cell (Cambridge, Massachusetts) 185(8): 1431-1443.e16; Javdan, S, B. and Deans, T. L. 2021. “Design and Development of Engineered Receptors for Cell and Tissue Engineering,” Online, Current Opinion in Systems Biology (London, England) 28:10036).

“Researchers at Uppsala University and KTH Royal Institute of Technology have developed a new form of precision medicine, an antibody, with the potential to treat several types of cancer” . . . the aim of the study was to establish a more flexible, faster and safer treatment for cancer than those currently available. The study has already shown that the method has the potential to be customized for each patient, thereby strengthening the immune system against cancer,” (Science X Staff 2024. “Multifunctional Antibody Shows Promise for Treating Several Types of Cancer,” Online, Online, São Paulo, Brazil: Medical Xpress; see also Mebrahtu, A., Lauren, I., Veerman, R., Guklular Akpinar, G., Lord, M., and 10 others 2024. “A Bispecific CD40 [cluster of differentiation 40] Agonistic Antibody Allowing for Antibody-peptide Conjugate Formation to Enable Cancer-specific Peptide Delivery, Resulting in Improved T Proliferation and Anti-tumor Immunity in Mice,” Online, Nature Communications (London, England) 15:9522).

Of considerable potential importance is the advent of means for overcoming resistance to and reinstating the efficacy of antibiotics dissipated by pathogens able to adapt to the original formation thereof (Science X Staff 2024. “Antibacterial Material Restores the Efficacy of Antibiotics against Resistant Bacteria,” Online, São Paulo, Brazil: Medical Xpress; Stepulane, A., Rajasekharan, A. K., and Andersson, M. 2024. “Antibacterial Efficacy of Antimicrobial Peptide-functionalized Hydrogel Particles Combined with Vancomycin and Oxacillin Antibiotics,” Online, International Journal of Pharmaceuties (Amsterdam, North Holland, Netherlands) 664:124630; Lennard, P. R., Hiemstra, P. S., and Nibbering, P, H. 2023. “Complementary Activities of Host Defence Peptides and Antibiotics in Combating Antimicrobial Resistant Bacteria,” Online, MDPI [Multidisciplinary Digital Publishing Institute] Antibiotics (Basel, Switzerland) 12(10): 1518).

In that the ability of a pathogen to form a biofilm can serve as a significant factor in its resistance to antibiotics, a related finding of potential importance are means for disrupting bacterial biofilms (Subrahmanyam, R. 2024. “Scientists Use Cow Gut Enzyme to Disrupt Bacterial Biofilms,” Online, Isle of Man, England: Phys.org; Ramakrishnan, R., Nair, A. V., Parmar, K., Rajmani, R. S., Chakravortty, D., and Das, D. 2024. “Combating Biofilm-associated Klebsiella pneumoniae Infections Using a Bovine Microbial Enzyme,” Online, NPJ [Nanjang Partner Journals] Biofilms and Microbiomes 10(1): 119; de Oliveira Junior, N, G. and Franco, O. L. 2020. “Promising Strategies for Future Treatment of Klebsiella pneumoniae Biofilms,” Future Microbiology (Milton Park, Oxfordshire, England) 15:63-79; Wang, G., Zhao, G, Chao, X., Xie, L., and Wang, H. 2020. “The Characteristic of Virulence, Biofilm and Antibiotic Resistance of Klebsiella pneumoniae,” Online, MDPI [Multidisciplinary Digital Publishing Institute] International Journal of Environmental Research and Public Health (Basel, Switzerland) 17(17): 6278).

“A team of researchers has found a quicker and more efficient way to create nucleoside analogs, a type of small molecule that can be used in treatments for everything from cancer to viral diseases., Nucleoside analogs are among the most important molecules to the advancement of modern medicine,” says the corresponding author of a paper in the journal Nature Communications describing the new process . . . “They're used as antivirals, as cancer therapeutics, in gene therapy,” (MacPherson, A. 2024. “Scientists Streamline Creation of Nucleoside Analogs, a Group of Life-saving Molecules,” Online, Isle of Man, England: Phys.org; Nuligonda, T., Kumar, G., Wang, J. W., Rajapaksha, D., Elayan, I. A., and 6 others 2024. “An Enantioselective and Modular Platform for C4′-modified Nucleoside Analogue Synthesis Enabled by Intramolecular Trans-acetalizations,” Online, Nature Communications (London, England) 15(1): 7080).

A semisynthetic advanced generation lactone ketolide macrolide antibiotic now long under test, nafithromycin, has been established to significantly exceed the therapeutic efficacy and scope of resistant bacteria susceptibility of azithromycin. In addition to the security lent by the automatic release of the drug as scheduled must be added the precise targeting made possible by directly piped delivery to the source.

In terms of research as to structure, safety, and efficacy, nafithromycin has an established legacy (Clarance, D. 2024. “Antibiotic Breakthrough from India Could Counter Drug-resistant Bacteria” Online, Noida, Uttar Pradesh, India: India Today; Butler, M. S., Henderson, I. R., Capon, R. J., and Blaskovich, M, A. T. 2024. “Antibiotics in the Clinical Pipeline as of December 2022,” Online, Journal of Antibiotics (Tokyo, Japan) 76(8): 431-473; erratum; 77(1): 71; Zhou, M., Wu, L., Kang. W., Li, Y., Zhang, G., and 6 others 2022. “In vitro Activity of Lactone Ketolide Nafithromycin (WCK 4873) against Streptococcus pneumoniae Isolates Enriched with Macrolide-resistance Phenotype Collected from Mainland China,” Online, JAC [Journal of Antimicrobial Chemotherapy]-Antimicrobial Resistance (Oxford, England) 4(5): dlac 103; Chavan, R., Zope. V., Chavan, N., Patil, K., Yeole. R., Bhagwat. S., and Patel, M. 2021. “Assessment of the In vitro Cytochrome P450 (CYP) Inhibition Potential of Nafithromycin, a Next Generation Lactone Ketolide Antibiotic,” Xenobiotica (London, England) 51(3): 251-261; Kohlhoff, S. and Hammerschlag, M, R. 2021. “In Vitro Activity of Nafithromycin (WCK 4873) against Chlamydia pneumoniae,” Online, Antimicrobial Agents and Chemotherapy (Washington, D.C.) 65(9): e0058521; Veeraraghavan, B., Varghese, R., Saigal, K., Balasubramanian, S., Bai, P. S. P., and 10 others 2021. “Activity of Novel Lactone Ketolide Nafithromycin against Multicentric Invasive and Non-invasive Pneumococcal Isolates Collected in India,” JAC [Journal of Antimicrobial Chemotherapy]—Antimicrobial Resistance (Oxford, England) 32): dlab066; Mahama, Q., Songuigama, C., and N. G. Deto, Jean-Paul 2020. “Pharmacochemical Aspects of the Evolution from Erythromycin to Neomacrolides, Ketolides and Neoketolides,” Online, Open Journal of Medicinal Chemistry (Glendale, California) 10(3) September 2020; Iwanowski, P., Bhatia, A., Gupta, M., Patel, A., Chavan, R., Yeole, R., and Friedland, H, D. 2019. “Safety, Tolerability and Pharmacokinetics of Oral Nafithromycin (WCK4873) after Single or Multiple Doses and Effects of Food on Single-dose Bioavailability in Healthy Adult Subjects,” Online, Antimicrobial Agents and Chemotherapy (Washington, D.C.) 63(12): e01253-19; Flamm, R. K., Rhomberg, P. R., and Sader, H. S. 2017. “In Vitro Activity of the Novel Lactone Ketolide Nafithromycin (WCK 4873) against Contemporary Clinical Bacteria from a Global Surveillance Program,” Online, Antimicrobial Agents and Chemotherapy (Washington, D.C.) 61(12): e01230-17; Rodvold, K. A., Gotfried, M. H., Chugh, R., Gupta, M., Friedland, H. D., and Bhatia, A. 2017. “Comparison of Plasma and Intrapulmonary Concentrations of Nafithromycin (WCK 4873) in Healthy Adult Subjects,” Online, Antimicrobial Agents and Chemotherapy (Washington, D. C.) 61(9): e01096-17; Waites, K. B., Crabb, D. M., and Duffy, L, B. 2016. In Vitro Activities of Investigational Ketolide WCK 4873 (Nafithromycin) and Other Antimicrobial Agents against Human Mycoplasmas and Ureaplasmas, Online, meeting abstract Sunday-480. Boston, Massachusetts: American Society for Microbiology, Washington D. C.).

An apparently novel strategy for overcoming bacterial resistance is to target [paraphrased] bacterial defenses against external stress for efficient antibiotic treatments; “The bacteria's primary defense against their environment is an intact cell envelope, and changes to the envelope can be crucial for the cell's survival. This makes the cell envelope an ideal target for future treatments of bacterial infections.”) Science X staff 2024. “Targeting Bacterial Defense Mechanisms for Effective Antibiotic Treatment,” Online, Isle of Man, England: Phys.org; Gray, D. A., Wang, B., Sidarta, M., Comejo, F. A., Wijnheijmer, J., and 6 others 2024. “Membrane Depolarization Kills Dormant Bacillus subtilis Cells by Generating a Lethal Dose of ROS [reactive oxygen species],” Online, Nature Communications (London, England) 15(1): 6877).

The eschewment of accretion such as of clot in side-entry devices and substrate tubing along the vascular tree and crystal along the urinary tract imperative for life-long sustainment of the system, numerous means for coating and lining these have been indicated in the specification. Now a new blood vessel-like coating has been developed that mimics the natural behavior of blood vessels in preventing clots. The avoidance of anticoagulants and the need for the periodic release of a heparin, for example, would eliminate the risk of several serious complications (Ji H, Yu K, Abbina, S., Xu, L., Xu, T., and 13 others 2024. “Antithrombotic Coating with Sheltered Positive Charges Prevents Contact Activation by Controlling Factor XII-biointerface Binding,” Online, Nature Materials (London, England) [in press, 12 November]; Beech, S. 2024. “New Blood Vessel-like Coating Could Make Medical Devices Safer,” Online at multiple sites to Health and Wellness|gisentinel.com).

Research in Nottingham England has found that “, peptide molecules that can guide key processes taking place during the natural healing of tissues [can be used] to create living materials that enhance tissue regeneration., The team developed a self-assembling methodology where synthetic peptides are mixed with whole blood taken from the patient to create a material that harnesses key molecules, cells, and mechanisms of the natural healing process. In this way, it was possible to engineer regenerative materials capable of not only mimicking the natural R H, but also enhancing its structural and functional properties.

“These materials can be easily assembled, manipulated, and even 3D printed while maintaining normal functions of the natural R H, such as normal platelet behavior, generation of growth factors, and recruitment of relevant cells important for healing. With this method, the team has shown the capacity to successfully repair bone in animal models using the animal's own blood,” (Padilla-Lopategui, S., Ligorio, C., Bu, W., Yin, C., Laurenza, D., and 6 others 2024. “Biocooperative Regenerative Materials by Hamessing Blood-clotting and Peptide Self-assembly,” Online, Advanced Materials (Weinheim, Germany) [in press] November 14: e2407156; Science X Staff 2024. “Scientists Transform Blood into Regenerative Materials, Paving the Way for Personalized, 3D-printed Implants,” Online, Isle of Man, England: Phys.org). Three dimensional printing is also addressed below in section 2b(1)(b)(b11), entitled Metered Compound Vascular Bypass, or Switched-organ Heterotopic Intact Allograft Thyroid and Parathyroid Transplantation

Access to the brain for the direct pipe-targeted release of drugs is by means of ductus side-entry valves on the internal carotids as shown in FIGS. 25A and 25B. Unlike confinement to the clinic, the master control microprocessor governing the release of medication to be targeted thus can be programmed to release the medication on a scheduled basis responsive to sensors indicating indicia associated with the mental state of the patient at any location remote from the clinic.

Specifically, Swedish researchers have found that protumoral lipid droplet-loaded macrophages, or foam cells, are enriched in human glioblastoma which facilitate growth in the tumor, and that these can be therapeutically targeted using drugs previously developed to treat arteriosclerosis and thus disrupting the ability of the foam cells to accumulate fat. “ . . . we found that TAFs [tumor-associated foam cells] exhibit distinct protumorigenic characteristics related to hypoxia, mesenchymal transition, angiogenesis, and impaired phagocytosis, and their presence correlates with worse outcomes for patients with glioma., Disrupting lipid droplet formation to target TAFs may represent an avenue for future therapeutic development for glioblastoma” (Governa, V., de Oliveira, K. G., Bang-Rudenstam, A., Offer, S., Cerezo-Magaña, M., and 25 others 2024. “Protumoral Lipid Droplet-loaded Macrophages are Enriched in Human Glioblastoma and Can Be Therapeutically Targeted,” Science Translational Medicine (Washington, D.C.) 16(771): eadk1168; Science X Staff 2024. ““Foam Cells' that Accelerate Tumor Growth in Glioblastoma Can Be Inhibited, Researchers Discover,” Online, Isle of Man, England: Medical Xpress).

Researchers at the University of Queensland have also discovered a way to pass through the blood brain barrier. “ . . . an essential nutrient called choline is transported into the brain by a protein called FLVCR2 [feline leukemia virus subgroup C cellular receptor family, member 2 encoded by the FLVCR2 gene also known as MFSD7B or SLC49A1] . . . “We used high-powered cryo-electron microscopes to see exactly how choline binds to FLVCR2., Choline sits in a cavity of FLVCR2 as it travels across the blood-brain barrier and is kept in place by a cage of protein residues (Science X Staff 2024. “Research on How Dietary Choline Travels through the Blood-brain Barrier Reveals Pathway for Treating Brain Disorders,” Online, Isle of Man, England: Medical Xpress; Cater, R. J., Mukherjee, D., Gil-Iturbe, E., Erramilli, S. K., Chen, T., and 18 others 2024. “Structural and Molecular Basis of Choline Uptake into the Brain by FLVCR2,” Nature (Basingstoke, England) 629(8012): 704-709).

Another breakthrough in crossing the blood-brain barrier is the development of a novel “ . . . blood-brain barrier-crossing conjugate which can deliver therapeutic molecules directly into the brain through a simple intravenous injection. The breakthrough comes from a specialized biological process called γ-secretase-mediated transcytosis. By attaching genetic tools to a compound called BCC10 [blood brain barrier crossing conjugate 10], researchers successfully delivered therapeutic molecules into mouse brains. In experiments using mice with amyotrophic lateral sclerosis, they dramatically reduced levels of disease-causing genes, demonstrating the method's potential effectiveness,” (StudyFinds Staff 2024. “Scientists Finally Unlock the Brain's Mighty Barrier to Treat Neurological Diseases,” Baltimore, Maryland: StudyFinds; Wang, C., Wang, S., Xue, Y., Zhong, Y., Li, H., and 15 others 2024. “Intravenous Administration of Blood-brain Barrier-crossing Conjugates Facilitates Biomacromolecule Transport into Central Nervous System,” Nature Biotechnology (New York, New York) In Press 2024 Nov. 25; Thompson, D. 2024. “Scientists Discover Way to Deliver Medicines across Brain's Protective Barrier,” Online at https://www.upi.com/Science News/2024/11/27/scientists-medicines-blood-brain-barrier/2591732726180, Boca Raton, Florida: United Press International). A caveat is that results obtained from work on murids often fails to translate to humans.

Addtionally, “Researchers at the Icahn School of Medicine at Mount Sinai have developed an innovative approach—demonstrated in mouse models and isolated human brain tissue—to safely and effectively deliver therapeutics into the brain, providing new possibilities for treating a wide range of neurological and psychiatric diseases., The blood-brain barrier-crossing conjugate (BCC) system platform takes advantage of a specialized biological process called γ-secretase-mediated transcytosis to deliver large therapeutic molecules, like oligonucleotides and proteins, directly into the brain through a simple intravenous injection. “(Science X Staff 2024. “Study Reveals New Way to Unlock Blood-Brain Barrier, Potentially Opening Doors to Treat Brain and Nerve Diseases,” Online, Isle of Man, England: Medical Xpress; Wang, C., Wang, S., Xue, Y., Zhong, Y., Li, H., and 15 others 2024. “Intravenous Administration of Blood-Brain Barrier-crossing Conjugates Facilitate Biomacromolecule Transport into Central Nervous System,” Nature Biotechnology (New York, New York) [in press] November 25).

“University of Pennsylvania engineers have modified lipid nanoparticles (LNPs)—the revolutionary technology behind the COVID-19 mRNA vaccines—to not only cross the blood-brain barrier but also to target specific types of cells, including neurons., This breakthrough marks a significant step toward potential next-generation treatments for neurological diseases like Alzheimer's and Parkinson's . . . [paraphrasing] The researchers demonstrate how peptides can serve as precise targeting molecules, enabling lipid nanoparticles to deliver mRNA specifically to the endothelial cells that line the blood vessels as well as the neurons of the brain (Scheffler, I. 2024. “Unlocking the Brain: Peptide-guided Nanoparticles Deliver mRNA to Neurons,” Online, Isle of Man, England: Phys.org.; Han, E, L., Tang, S., Kim, D., Murray, A. M., Swingle, K, L., and 6 others 2024. “Peptide-functionalized Lipid Nanoparticles for Targeted Systemic mRNA Delivery to the Brain,” Online, [American Chemical Society] Nano Letters (Washington, D.C.).

“University of California at San Francisco scientists have developed a “molecular GPS” to guide immune cells into the brain and kill tumors without harming healthy tissue. It is the first living cell therapy that can navigate through the body to a specific organ, addressing what has been a major limitation of CAR-T [chimeric antigen receptors/chimeric immunoreceptors, chimeric T cell receptors/artificial T cell receptors]-T cancer therapies until now. The technology worked in mice and the researchers expect it to be tested in a clinical trial next year . . . the scientists showed how the immune cells could eliminate a deadly brain tumor called glioblastoma—and prevent recurrences,” (Gadye, L. 2024. “Molecular ‘ZIP Code’ Draws Killer T Cells to Brain Tumors,” Online, Isle of Man, England: Medical Xpress; Simic, M. S., Watchmaker, P. B., Gupta, S., Wang, Y., Sagan, S. A., and 18 others 2024. “Programming Tissue-sensing T Cells that Deliver Therapies to the Brain,” Science (Washington, D.C.) 386(6726): eadl 4237).

An essential enzyme cofactor for the growth and development of cells, malignant cells grow and proliferate much more rapidly and therefore need more copper than do normal cells. [paraphrased] The researchers attached a component to the copper-binding domains of the chaperone Atox1 peptide that promotes its uptake into tumor cells, as well as an additional component “that ensures the individual peptide molecules aggregate into nanofibers once they are inside the tumor cells.

In this form, the fiber surfaces have many copper-binding sites in the right spatial orientation to be able to grasp copper ions from three sides with thiol groups (chelate complex)” (Jeena, M. T., Link, J, Zhang, J., Harley, I., Turunen, P., and 10 others 2024. “Chaperone-derived Copper (I)-binding Peptide Nanofibers Disrupt Copper Homeostasis in Cancer Cells,” Online, Angewandte Chemie (International edition in English) October 24: e202412477; Science X Staff 2024. “Nanofibers Made of Copper-binding Peptides Disrupt Cancer Cells,” Online, Isle of Man, England: Phys.org).

The use of light to create electrically conductive materials directly in the body should overcome the problem of the need for the tissues surrounding an implanted electrode to adapt to the electrode with obstructive results, possibly advancing the functionality of implanted electrodes for many purposes, as well for use in optogenetics (Ek, F., Abrahamsson, T., Savvakis, M., Bormann, S., Mousa, A. H., and 12 others 2024. “In vivo Photopolymerization: Achieving Detailed Conducting Patterns for Bioelectronics,” Adv Sci (Weinheim, Germany) November 7: e2408628, Online at https://onlinelibrary.wiley.com/doi/10.1002/advs.202408628; Science X Staff 2024. “Using Light to Create Bioelectronics Inside Living Organisms,” Online, Isle of Man, England: Phys.org).

“A biocooperative regenerative gel-like substance which can be 3D-printed uses synthetic peptides to improve the structure and function of the barrier naturally formed by blood when it clots has been prepared to be effective in repairing bone damage in murid subjects” (Nield, D. 2024. “Living Material Made From Blood Can Repair Bones, Study Shows,” Online, Canberra, Austrailia: Science Alert; Padilla-Lopategui, S., Ligorio, C, Bu, W., Yin, C., Laurenza, D, and 6 others 2024. “Biocooperative Regenerative Materials by Hamessing Blood-clotting and Peptide Self-assembly,” Online, Advanced Materials (Weinheim, Germany) 14: e2407156). Again, the translatability of results obtained with murids is unpredictable.

2a(1). Congenital Defects of the Heart and Other Organs: The Importance of Prompt Intervention

If the heart of a fetus or neonate fails to provide normal circulation, the rest of the body, to include the brain, lungs, and all other organs and tissues cannot develop normally. While less serious defects such as an atrial septal defect can be repaired by positioning a button obturator in the opening, to establish normal circulation, serious malformities such as a double outlet ventricle, univentricular heart, hypoplastic left heart syndrome, tetralogy of Fallot, and their numerous variants and associated defects cannot. A heart so congenitally defective that the best surgical repair would not initiate normal circulation will result in global developmental degradation and lifelong impairment.

Too often, repair will only have sustained the severely heart impaired neonate for a sick and progressively deteriorating decade or so before death with no prospect for reaching adulthood, much less having done so with the mental acuity essential to make a significant accomplishment. The interdependency among the organs is reflected throughout life in the global deterioration that ensues when any one organ begins to fail, heart, kidney, and liver failure representing familiar examples. Congenital heart defects are associated with low birth weight, stillbirth, early death, failure to thrive, and reduced life span with life-long morbidity. That all current measures for dealing with severe congenital heart defects whether surgical, mechanical, or both combined fail to allow normal academic performance is dealt with below in this section.

By the same token, as a fundamentally less traumatic operation than that conventional, a compound vascular bypass heart transplant in a perinate to interdict this degradation would be difficult enough to risk the outcome and costly. This is due to the minuteness of the vessels and the cost to provide the parts necessary to perform the operation. To overcome this impasse, the heart transplant is initially reduced in difficulty and expense by grouping the tiny vessels which in an adult would be separately valved, to flow past and be diverted by a few common valves. Significantly reducing the duration of the operation for the already impaired patient, the odds for a favorable outcome are much improved.

This interim measure uses parts with sufficiently internal elasticity to accommodate growth until the patient with a new heart is ready to have the vessel grouping devices removed and the vessels incurrent into and excurrent out of the grouping devices directly anastomosed with the aid of a bypass. Performed through small incisions without cardiopulmonary bypass, cardioplegia, or general anesthesia, this follow-up procedure is even less traumatizing than the initial compound vascular bypass heart transplant of which the entry wound is the only significant cause of trauma.

Grouping is shown in FIGS. 15C thru 15E and addressed below in section 2b(1)(b)(b14), entitled Confluence Conduits: Connection of One or More Bloodlines to Flow through a Common Channel to the Same or a Different Number of Bloodlines with Self-leveling Apportionment of Outflow, in the subsection thereof entitled Tiny Vessel Grouped Inlet/Outlet Expandable Confluence Conduits, and in section 6, entitled Description of the Preferred Embodiments of the Invention, in the subsection thereof entitled Tiny Vessel Grouping Device.

Tiny vessel grouping is an interim expedient devised to avert the need to apply a valve to each of the small vessels in a young pediatric patient. After an interval during which the baby is allowed to grow and gain in strength, the grouping devices are removed without an interruption in blood flow by bypass each vessel as depicted in FIG. 15D. When a larger autologous vessel segment is available, to anastomose the grouped small vessels to a single bypass is also an option but does not gain time. In FIGS. 15A, 15C, and 15D, the appearance of connection to the supraaortic arteries is meant as exemplary any other three or more arteries no less applicable.

Accordingly, here the subject is an improved method of solid organ transplantation. Of particular interest is an improved method for replacing any solid organ before the failure of that organ to support every other organ and tissue in the body results in global degeneration. The interdependence of the parts of the body commences in utero, so that defective organs are best identified and replaced as early as possible. Accordingly, the method should facilitate organ replacement in penates and neonates. Inadequate repair and mechanical devices can usually accomplish little more than to stall death. A severely defective heart not only fails the rest of the body directly through perfusion inadequacy but often indirectly as well, by causing difficulties in feeding and swallowing, sometimes iatrogenic, and therewith, inadequate growth rate, weight gain, and failure to thrive.

Malnutrition in infants with a defective heart has been intensively studied (see, for example, Mumi, I. K., Patmasari, L., Wirawan, M. T., Arafuri, N., Nurani, N., and 3 others 2023. “Outcome and Factors Associated with Undernutrition among Children with Congenital Heart Disease,” Online PLoS (Public Library of Science) One 18(2): e0281753; Onesimo, R., Sforza, E., Giorgio. V., Rigante, D., Kuczynzka, E., and 7 others 2023. “Predicting the Clinical Trajectory of Feeding and Swallowing Abilities in CHARGE [Coloboma, Heart defects, Atresia choanae, Retarded growth, malformed Genitalia, and statoacoustic and olfactory impairment of the Ear nose respectively] Syndrome,” Online, European Journal of Pediatrics Feb. 17, 2023 (in press); Aryafar, M., Mahdavi, M., Shahzadi. H., and Nasrollazadeh, J. 2022. “Effect of Feeding with Standard or Higher-density Formulas on Anthropometric Measures in Children with Congenital Heart Defects after Corrective Surgery: A Randomized Clinical Trial,” European Journal of Clinical Nutrition 76(12): 1713-1718; Kwa, L., Willette. S., and Schroeder, J. W. Jr. 2022. “Evaluating Dysphagia in Infants with Congenital Heart Disease Using Fiberoptic Endoscopic Evaluation of Swallowing,” International Journal of Pediatric Otorhinolaryngology 152:111004; Narawane, A., Rappazzo, C., Hawney, J., Clason, H., D. J., and Ongkasuwan, J. 2022. “Vocal Fold Movement and Silent Aspiration after Congenital Heart Surgery,” Laryngoscope 132(3): 701-705; Norman. V., Zühlke, L., Murray, K., and Morrow, B. 2022. “Prevalence of Feeding and Swallowing Disorders in Congenital Heart Disease: A Scoping Review,” Online, Frontiers in Pediatrics 10:843023; Pettigrew, J., Tzannes, G., Swift, L., Docking, K., Osland, K., and Cheng, A. T. 2022. “Surgically Acquired Vocal Cord Palsy in Infants and Children with Congenital Heart Disease (CHD): Description of Feeding Outcomes,” Dysphagia 37(5): 1288-1304; Singal, A., Sahu, M. K., Trilok Kumar, J., and Kumar, A. 2022. “Effect of Energy and/or Protein-dense Enteral Feeding on Postoperative Outcomes of Infant Surgical Patients with Congenital Heart Disease: A Systematic Review and Meta-analysis,” Nutrition in Clinical Practice 37(3): 555-566; Chinawa, A. T., Chinawa, J. M., Duru, C. O., Chukwu, B. F., and Obumneme-Anyim, I. 2021. “Assessment of Nutritional Status of Children with Congenital Heart Disease: A Comparative Study,” Online, Frontiers in Nutrition 17:644030; Shahi, N., Philliips, R., Meier, M., Shirek. G., Goldsmith, A., and 3 others 2021. “Gastrostomy Button Placement in Infants with Cyanotic versus Acyanotic Congenital Heart Disease,” Journal of Surgical Research 259:407-413: Souza, P. C., Gigoski, V. S., Etges, C, L., and Barbosa, L. D, R. 2018. “Findings of Postoperative Clinical Assessment of Swallowing in Infants with Congenital Heart Defect,” (in English and Portuguese), Online, CoDas 30(1): e20170024; Blazquez, A., Clouzeau, H., Fayon, M., Mouton, J. B., Thambo, J. B., Enaud, R., and Lamireau, T. 2016. “Evaluation of Nutritional Status and Support in Children with Congenital Heart Disease,” European Journal of Clinical Nutrition 70(4): 528-531; da Rosa Pereira, K., Firpo, C., Gasaparin, M., Teixeira, A. R., Dornelles, S., Bacaltchuk. T., and Levy, D. S. 2015. “Evaluation of Swallowing in Infants with Congenital Heart Defect, Online, International Archives of Otolaryngology 19(1): 55-60; Hassan, B. A., Albanna, E. A., Morsy. S. M., Siam, A. G., Al Shafie, M. M., and 4 others 2015. Nutritional Status in Children with Un-operated Congenital Heart Disease: An Egyptian Center Experience,” Online, Frontiers in Pediatrics 3:53: Yi. S.-H., Kim, S.-J., Huh, J., Jun, T.-G., Cheon, H. J., and Kwon, J.-Y. 2013. “Dysphagia in Infants after Open Heart Procedures,” American Journal of Physical Medicine and Rehabilitation 92(6): 496-503; Ratanachu-Ek, S. and Pongdara, A. 2011. “Nutritional Status of Pediatric Patients with Congenital Heart Disease Pre- and Post Cardiac Surgery,” Journal of the Medical Association of Thailand 94 Supplement 3: S133-S137; Jadcherla, S. R., Vijayapal, A. S., and Leuthner, S. 2009. “Feeding Abilities in Neonates with Congenital Heart Disease: A Retrospective Study,” Online, Journal of Perinatology 29(2): 112-118; Thommessen, M., Heiberg. A., and Kase, B, F. 1992. “Feeding Problems in Children with Congenital Heart Disease: The Impact on Energy Intake and Growth Outcome,” European Journal of Clinical Nutrition 46(7): 457-464).

The utility of a disorder control system implanted in a gravida, her fetus, and placenta in the finer diagnosis and treatment of intrauterine growth restriction is also addressed in sections 2a, entitled Background of the Invention and 2b(2)(c), entitled Vascular Segment Prostheses: Inline Coupling Jackets and Bridging, or Tie-lines.

Reduced fetal head growth with neurodevelopmental delay can result due to true, or symmetrical, intrauterine growth restriction, also known as global growth restriction, or asymmetrical, whether due to placental insufficiency, altered intrauterine brain hemodynamics, or due to a congenital heart defect, or to any or all of these combined (see, for example, Calek, E., Binder, J., Palmrich, P., Eibensteiner, F., Thajer, A., and 4 others 2023. “Effects of Intrauterine Growth Restriction (IUGR) on Growth and Body Composition Compared to Constitutionally Small Infants,” Online, MDPI [Multidisciplinary Digital Publishing Institute] Nutrients (Basel, Switzerland) 15(19): 4158; Sharma, D., Sharma, P., and Shastri, S. 2017. “Genetic, Metabolic and Endocrine Aspect of Intrauterine Growth Restriction: An Update,” Journal of Maternal-Fetal and Neonatal Medicine (Boca Raton, Florida) 30(19): 2263-2275; Sharma, D., Shastri, S., and Sharma, P. 2016. “Intrauterine Growth Restriction: Antenatal and Postnatal Aspects,” Online, Clinical Medicine Insights. Pediatrics (Thousand Oaks, California) 14:10:67-83; Murray, E., Fernandes, M., Fazel, M., Kennedy, S. H., Villar, J., and Stein, A. 2015. “Differential Effect of Intrauterine Growth Restriction on Childhood Neurodevelopment: A Systematic Review,” British International Journal of Obstetrics and Gynaecology (Oxford, England) 122(8): 1062-1072). Such a system can signal an inadequacy of fetal oxygen and/or nutrition, and equipped with the responsive means exogenous or endogenous within the umbilical cord/and/or the fetus, as well as maternal sensors and responsive means, can be programmed to counteract the insufficiency.

In either case, the implantation of a prosthetic disorder response system in the gravida with sensors placed in the fetus or fetuses and placenta placentas whether separate, fused, or single, associated or not with feto-fetal transfusion syndrome, or twin oligohydramnios-polyhydramnios sequence, as directed by the perinatologist on the basis of preliminary diagnostics as well can diagnose and treat numerous adverse consequences of the condition. Because intrauterine growth restriction can be due to myriad factors, the sensors and point-of-need counteractant release provided by an implanted disorder response can afford a powerful mechanism for better characterizing the condition in detail to reduce what otherwise could prove bewildering. Shortly after delivery, the neonate or neonates are implanted with an automatic disorder response system and future pregnancies are ruled out, components of the system considered nonessential postpartum can be removed.

The utility of an automatic fully implanted disorder response system in the diagnosis and treatment of intrauterine growth restriction is also addressed below in section 2b(2)(c), entitled Vascular Segment Prostheses: Inline Coupling Jackets and Bridging, or Tie-lines (see, for example, Chew. L, C. and Verma, R. P. 2023. “Fetal Growth Restriction,” Online, Treasure Island, Florida: StatPearls Publishing Co.; Dapkekar, P., Bhalerao, A., Kawathalkar, A., and Vijay, N. 2023. “Risk Factors Associated with Intrauterine Growth Restriction: A Case-control Study,” Cureus (Palo Alto, California) 15(6): e40178; van Uden, L. and Tchirikov. M. A 2023. “Study of the Literature on Intrauterine Treatment Options for Chronic Placental Insufficiency with Intrauterine Growth Restriction Using Intrauterine Intravascular Amino Acid Supplementation,” MDPI [Multidisciplinary Digital Publishing Institute] Life (Basel, Switzerland) 23:13(6): 1232; Aliasi. M., Snoep. M. C., van Geloven, N. and Haak, M, C. 2022. “Birthweight and Isolated Congenital Heart Defects-A Systematic Review and Meta-analysis,” Online, British Journal of Obstetrics and Gynecology 129(11): 1805-1816; Parekh, S. A., Cox, S. M., Barkovich, A. J., Chau, V., Steurer, M. A., and 4 others 2022. “The Effect of Size and Asymmetry at Birth on Brain Injury and Neurodevelopmental Outcomes in Congenital Heart Disease,” Online, Pediatric Cardiology 43(4): 868-877; van Nisselrooij, A, E, L., Jansen, F, A. R., van Geloven, N., Linskens, I. H., Pajkrt, E., and 6 others 2020. “Impact of Extracardiac Pathology on Head Growth in Fetuses with Congenital Heart Defect,” Online, Ultrasound in Obstetrics and Gynecology 55(2): 217-225; Mebius, M. J., Clur, S, A. B., Vink, A. S., Pajkrt, E., Kalteren, W. S., and 4 others 2019. “Growth Pattems and Cerebroplacental Hemodynamics in Fetuses with Congenital Heart Disease,” Online, Ultrasound in Obstetrics and Gynecology 53(6): 769-778; Alsaied, T., Tseng, S., King, E., Hahn, E., Divanovi, A. Habli, M., and Cnota, J. 2018. “Effect of Fetal Hemodynamic on Growth in Fetuses with Single Ventricle or Transposition of the Great Arteries,” Online, Ultrasound in Obstetrics and Gynecology 52(4): 479-487; Jansen, F, A. R., van Zwet, E. W., Rijaarrsdam, M. E., Pajkrt, E., van Velzen, C, L., and 7 others 2016. “Head Growth in Fetuses with Isolated Congenital Heart Defects: Lack of Influence of Aortic Arch Flow and Ascending Aorta Oxygen Saturation,” Online, Ultrasound in Obstetrics and Gynecology 48(3): 357-364; Bamfo, J, E. and Odibo, A. O. 2011. “Diagnosis and Management of Fetal Growth Restriction,” Online, Journal of Pregnancy (Cairo, Egypt) 2011:640715; additional references cited above in section 2a, entitled Background. General, and below in section 2b(2)(c), entitled Vascular Segment Prostheses: Inline Coupling Jackets and Bridging, or Tie-lines).

The suspected better adaptability of the neonate to the direct connections of an accessory heart in the chest, or if necessary, in the abdomen to the defective native heart is mentioned above in section 2a, entitled Background. General. The plasticity of the neonatal circulatory system responsive to the changes it undergoes during normal development is indicative of its ability to adapt in general and to adapt to structural changes introduced in reconstructive surgery to correct congenital cardiac defects, to include those most serious, such as a univentricular heart, or tetralogy of Fallot (see, for example, Vrancken, S, L., van Heijst, A. F., and de Boode, W. P. 2018. “Neonatal Hemodynamics: From Developmental Physiology to Comprehensive Monitoring,” Online, Frontiers in Pediatrics 6:87; Bloomekatz, J., Galvin-Santisteban, M., and Chi, N, C. 2016. “Myocardial Plasticity: Cardiac Development, Regeneration, and Disease,” Online, Current Opinion in Genetics and Development 40:120-130).

Pertinent to the different schemes presented below for positioning and connecting a second, or accessory, heart to assist or replace and assist that native in a neonate with a congenital heart defect, this factor is likely pertinent but to a much lesser degree in older patients with end stage heart failure. Where a compound vascular bypass or switched-organ heart transplant to be described will conclusively eradicate the defective circulation of the infant heart with a severe congenital defect that would have led to global deficits in development, conventional repair involves reconstruction that may necessitate multiple operations. The repair of a hypoplastic left heart, for example, requires three operations—a Norwood, Glenn, and Fontan—over the course of up to the first five formative years of life during which normal circulation that would have precluded maldevelopment remained lacking.

Moreover, once surgically repaired thus, the life of the infant will usually have been saved, but with circulation that remains abnormal and maldevelopment perpetuated. Neither does the use of a left ventricular assist device correct the problem. Univentricular (single ventricle, double inlet left ventricle), double outlet right ventricle, tetralogy of Fallot, and still other complex defects similarly defy satisfactory reconstruction and pose a ruined life and the constant risk of death-without a heart transplant to put an abrupt stop to the predicament, especially if the heart transplant is stripped of its conventional trauma and risk. The decision favoring repair through reconstruction using autologous tissue rather than performing a conventional heart transplant is based upon three factors.

A conventional heart transplant is severely traumatizing to both donor and recipient hearts and the baby; abortion rampant notwithstanding, the pool of available hearts is inadequate; and due to immunocompromise and the strict drug regimen that must be followed, a transplant is misconstrued as exchanging one illness for another. In fact, a fully implanted prosthetic disorder response system not only administers the transfer of the graft organ from the circulatory system of the donor maintained past death on life support into the circulatory system of the recipient, but continues to treat the patient automatically and indefinitely so that the use of drugs is optimized and administered in strict accordance with the system prescription-program.

Normal development in utero is dependent upon competent circulation and respiration (see, for example, Dossche, L. W. J., Kersten, C. M., Zanen-van den Adel, T., Wijnen, R. M. H., Gischler, S., and 3 others 2023. “Long-term Neurodevelopment in Children with Resected Congenital Lung Abnormalities,” Online, European Journal of Pediatrics (Berlin, Germany) 182(9): 3845-3855; Zaidi, A. H., Newburger, J. W., Wypij, D., Stopp, C., Watson, C. G., and 3 others 2018. “Ascending Aorta Size at Birth Predicts White Matter Microstructure in Adolescents Who Underwent Fontan Palliation,” Journal of the American Heart Association (Oxford, England) 7(24): e010395; Gaynor, J. W., Stopp, C., Wypij, D., Andropoulos, D. B., Atalla, J., and 23 others 2015. “Neurodevelopmental Outcomes after Cardiac Surgery in Infancy,” Pediatrics 135(5): 816-825; Sun, L., Macgowan, C. K., Sled, J. G., VII, S., Manlhiot, C., and 8 others 2015. “Reduced Fetal Oxygen Consumption is Associated with Smaller Brain Size in Fetuses with Congenital Heart Disease,” Circulation 131:1313-1323; Beca, J., Gunn, J. K., Coleman, L. Hope, A., Reed, P. W., and 5 others 2013. “New White Matter Brain Injury after Infant Heart Surgery is Associated with Diagnostic Group and the Use of Circulatory Arrest,” Circulation 127(9): 971-979).

Prenatal detection of abnormalities of the liver, gall bladder, and/or bile duct and the replacement or satisfactory repair thereof can ward off the deficit in normal growth that would otherwise ensue (see, for example, Chen, W., Geng, J., Tan, Y, L., Zhao, L., Jia, H., H., and Guo W. L. 2021. “Different Characteristics of Infants Diagnosed with Congenital Choledochal Malformation Prenatally or Postnatally,” Online, Scientific Reports (London, England) 11(1): 20; Shaughnessy, M. P., Spencer-Manzon, M., and Cowles, R, A. 2020. “Antenatally Detected Liver and Biliary Pathology,” Seminars in Pediatric Surgery (Philadelphia, Pennsylvania) 29(4): 150939; Ford, K. E., Cooper, L. R., and Davenport, M. 2015. “Growth in Children with Choledochal Malformations: Effect of the Roux Loop,” Pediatric Surgery International (Berlin, Germany) 31(11): 1015-1019).

Here the object is to initiate normal circulation, respiration, and/or other function as early as possible, thus averting the secondary deficiencies that follow deficient circulation at birth and after repair; even today a severely malformed heart can be repaired only to the extent that the infant does not die—not to the extent that deficiencies secondary to a congenitally defective heart can be averted. Conventional repair of a univentricular heart, for example, allows the developmental deficiencies caused by the faulty circulation that remains postoperatively to continue. For this reason, a heart severely defective at if not prior to birth should be replaced as soon as possible, the recommended method-because it involves far less trauma and no period of anoxia due to the use of the interruption in blood flow being compound vascular bypass transplantation, as will be described.

Current imaging allows the recognition of defects in development that arise in utero; however, adequate means for dealing with these are often wanting. The result following the inadequate repair as the only kind currently possible is that circulation will have been made closer to, but not normal. Repair which should be used only to sustain the patient until a proper end treatment can be performed is allowed to continue despite the progress in pathology long recognized to ensue. For example, serious defects in the development of the aorta may be discerned in the neonate where individualized surgical repair is the only option, one demanding inordinate skill and expertise likely to be available only at a remote medical center.

Compared to the consequences of impaired perfusion following repair, the lifelong need for immunosuppressives is a nuisance, but not consequential for development. A totally implanted automatic drug releasing system will ameliorate the nuisance, and immunosuppressive medication can be expected to advance toward improved efficacy. Such a system will provide the direct pipeline targeting of medication, minimizing exposure to nontargeted tissue and increasing susceptibility to infectious disease.

Except for excision and anastomosis of a small coarctation, if the thoracic aorta in a neonate or infant is malformed, then repair by a custom designed patch aortoplasty and/or the placement of an endostent unable to expand with growth is materially inferior to replacement with a prosthesis of the kind shown in FIGS. 36A thru 36D, 36F, and 37D devised so that precise measurement is unnecessary, to which the corollary is that a long period for growth follows placement.

Endostents can be made self-expandable and sufficiently elastic to support pulsatile flow, but not expandable to the extent that would accommodate rapid growth in a frail neonate and avert the need for early and probably multiple revisions. For a neonate, the prosthesis addressed below in section 2b(2)(d), entitled Anoxia-free Aortic Repair: Aortic Prostheses Thoracic, Thoracoabdominal, and Abdominal, is provided in a single spontaneously adapted-to fit size that eliminates unavailability and forestalls revision over a long period during which the infant can gain in strength.

For a neonate or infant, such a device has not been available, and were a version of the kind available for an adult made available, it would not expand to accommodate the rapid growth of a newborn. Moreover, its performance would depend upon the accuracy of the initial measurements supplied to the supplier, which consideration is no less valid for an adult. Surgical and/or mechanical repair of serious malformity should be undertaken not as an end treatment but rather with the object of sustaining the patient until a replacement heart can be found. With the legalized abortion of normal fetuses, normal replacement organs should be plentiful; however, no provision of law requires that a prospective abortion be denied pending receipt of written consent to organ harvesting.

What is currently referred to as a ‘heart transplant’ is in fact the replacing of the front of the recipient heart, to include the ventricles, with that of a deceased donor where the donor portion is obtained by cutting it out from the back of the donor heart, then stitched onto the back of the recipient heart. Transfer by this severely traumatic method is not direct but usually follows a period of donor heart storage in a cold static or flowing perfusate after which the donor heart has undergone the trauma of death and during which the heart deteriorates. Ex vivo machine transport allows the continuation of perfusion during transit, but the operative technique for replacing the heart is still severely injurious.

Repair suitable as a bridge to replacement with a sound heart, unless a replacement heart for a neonate or infant cannot be found, transplantation, not repair, should be undertaken. Addressed below in sections 2b(1)(b)(b2), entitled Prenatal Compound Vascular Bypass, or Sudden Switched-organ Transplantation of the Heart, and 2b(1)(b)(b3), entitled Neonatal and Infant Compound Vascular Bypass, or Sudden Switched-organ Transplantation of the Heart, the preferred method for transplanting any organ is fundamentally less traumatizing than is conventional heart transplantation; the switch method of solid organ transplantation, described below in section 2b(1), transfers the heart intact without incision from the donor kept on life support to the recipient bloodlessly and without interruption in the flow of blood.

Current methods employed in solid organ transplantation present two major flaws. The first is allowing the graft organ to deteriorate before placement, followed by placement that is severely traumatizing. The second is that the operative techniques are too complex to entrust to any but the most highly trained and experienced personnel limiting such operations to only the largest medical centers. Thus, replacement organs and the skill to use these are too often remote and in short supply, so that absent techniques for transplantation that could be performed by general surgeons and support personnel at local hospitals, even a significant increase in the number of graft organs available could not be exploited.

The switch, or compound vascular bypass, method of solid organ transplantation seeks to remedy both of these shortcomings by making it possible to replace organs in neonates and infants which are too malformed to be restored to normal function without needless trauma to the graft organ or the recipient and to do so by means of surgical techniques less complex as to allow the application thereof by general surgeons at community hospitals. Moreover, the correction is accomplished with minimal loss of blood, relatively little trauma, and quickly. Specifically, significant trauma is limited to the preparation of an open field and much less so to the intracorporeal procedure.

Accordingly, the prompt replacement of an irreversibly malformed organ averts the global secondary deterioration that would otherwise have been imposed upon the rest of the body. One example of the need for quick response—that prompt placement of a graft organ reduces the extent of deterioration to induce immune rejection by the recipient—has been addressed. With the switch technique for transplantation, the graft organ is not preserved ex vivo.

The circulatory system of the recipient is diverted from the diseased native organ of the recipient and redirected through that of the donor, the graft organ remaining intact throughout. When urgent, sudden compound vascular bypass, or sudden switched-organ transplantation accomplishes this transfer abruptly but in a manner that averts many drawbacks to the transplantation of a preserved graft organ, considerably increasing the odds for acceptance by the recipient. However, because the switch is sudden, the opportunity to incorporate immune tolerance induction into the transition process is passed over.

This does not mean that the manual infusion of hematopoietic stem cells and/or blood products, for example, cannot be carried out before or after the switch, or that conventional diagnostics are any the less applicable. Rather, the process is continuously driven by the master control microprocessor of an implanted hierarchical control system executing a prescription-program fed data from implanted sensors, some integrated into the side-entry jackets and connectors. This data directly drives the process of transition with an instantaneity that optimizes the rate of tolerance induction and quickly avoids adverse reactions. That is, based upon this feedback, the master controller instantaneously accelerates, pauses, or truncates the process, continuously optimizing the rate of tolerance induction.

Thus, to the minimal trauma of organ transfer provided by the sudden compound vascular bypass, or sudden switched-organ method, the metered switch method adds allotransplantation with continuously minimized immune suppression. When time allows, the metered switch method extends the transfer over an interval during which the gradual return of recipient blood having passed through the donor allows the blood of recipient and donor to blend, affording a measure of immune tolerance induction. During this process, less severe adverse immunological reactions are automatically responded to by the implanted automatic response system, which can stop the process if the reaction is serious as to presage rejection.

However, since preoperative tests are conducted to determine the degree of donor-recipient compatibility, the likelihood of this is considerably reduced. Most congenital heart defects, such as openings in the interatrial septum, are less severe and repairable to good effect as to dispel consideration of a transplant. Some defects, however, such as univentricular, cannot be repaired to the extent that circulation would then have been made normal. Fetal diagnostics is addressed below in section 2a(2), entitled Inventive Content-pertinent Fetal Diagnostics.

Because extracardiac maldevelopment due to a defective heart can be detected by the second trimester, the fetus with significant maldevelopment of the heart should be withdrawn long enough for a heart transplant, ideally using the compound vascular bypass switch method to implant a heart that had not been subjected to ischemia and preservation, and then returned for the resumption of an otherwise normal gestation (see, for example, Patkee, P. A., Baburamani, A. A., Kyriakopoulou, V., Davidson, A., Avini, E., and 6 others 2019. “Early Alterations in Cortical and Cerebellar Regional Brain Growth in Down Syndrome: An in Vivo Fetal and Neonatal MRI Assessment,” Neuroimage. Clinical (Amsterdam, North Holland, Netherlands) 25:102139; Peyvandi, S. and Donofrio, M. T. 2018. “Circulatory Changes and Cerebral Blood Flow and Oxygenation during Transition in Newborns with Congenital Heart Disease,” Seminars in Pediatric Neurology 28:38-47). The latter are better served by replacing the defective heart.

In pediatric cardiac transplantation, for example, advantages of the switch method over the conventional bicaval orthotopic technique is relative simplicity that eliminates intricate dissection and anastomosis, which inflicts considerable trauma on both donor and recipient hearts, when possible, implants a heart never having gone through ischemia or preservation, and limits the procedure to relatively few specialists. Replacement of the organ in a prenate even by a cardiac surgeon unaccustomed to microsurgical work is likely to achieve a less satisfactory outcome. That the graft organ remains intact thus expands the cohort of operators able to undertake such an operation. In neonatal heart repair and transplantation, ‘getting it right’ at the outset is crucial: imperfection at this time results in maldevelopment likely to affect the entire body and brain to the end of life—to deny the potential neurodevelopmental outcome and attainment that the infant would otherwise have realized.

A defective heart results in defective circulation which prevents the normal development of the entire body to include the brain. It is therefore of the utmost importance to stop the faulty circulation as soon as possible, (see, for example, Peyvandi, S., Latal, B., Miller, S. P., and McQuillen, P. S. 2019. “The Neonatal Brain in Critical Congenital Heart Disease: Insights and Future Directions,” NeuroImage 185:776-782; Karl, T. R., Cochrane, A., and Brizard, C. P. 1997. “Arterial Switch Operation. Surgical Solutions to Complex Problems,” Texas Heart Institute Journal 24(4): 322-333; Davis, A. M., Wilkinson, J, L., Karl, T. R., and Mee, R, B. 1993. “Transposition of the Great Arteries with Intact Ventricular Septum. Arterial Switch Repair in Patients 21 Days of Age or Older,” Journal of Thoracic and Cardiovascular Surgery 106(1): 111-115; additional references pertaining to maldevelopment in organs cited below in this section). The interdiction of body-wide maldevelopment at the outset of life is crucial.

However, the developmental period preceding delivery is susceptible to maldevelopment, and while transluminal procedures can be accomplished prior to birth, in severely defective fetal hearts these may allow repair of an otherwise fatal defect but fail to initiate normal development. While the heart continues to develop in utero, the fetus is first entirely, then largely, dependent upon the circulation of the mother. Self-sufficiency not essential, the heart is free to continue maldeveloping. In this circumstance, the only satisfactory solution is to replace a severely defective heart prior to birth, and because it eliminates the need to enter the heart, the switch approach facilitates heart transplantation at the microsurgical level.

Whether the plasticity of nerve can overcome congenital malformity of the heart with consequently impaired circulation depends upon the severity of the defect and the degree to which surgical repair can instate normal circulation. While spontaneous adaptation made possible by plasticity can effect improvement in extracardiac development, there is a limit to the degree of malformity that can be accommodated.

In cases of severe malformity such as hypoplastic left heart syndrome, a univentricular heart, or tetralogy of Fallot, for example, the patient often survives but is neuropsychologically impaired. This is so even if the development of the heart and the brain are not both under pleiotropic genetic control so that a perfect repair of the heart would still not allow normal development of the brain. The relevance of this to the subject matter of concern is that the combination of a pre- or neonatal compound vascular bypass heart transplantation and a prosthetic disorder response system to administer the procedure and provide follow-up treatment automatically is proposed as the optimal remedy for minimizing if not eliminating neurodevelopmental deficiencies secondary to a congenitally defective heart.

Severe congenital deformity of the heart in any genetic context exceeds the ability of neural plasticity and surgical skill to overcome (see, for example, Feng, Z., Chen, X., Li, T., Gao, H., Chen, W., and 7 others 2022. “An X-linked PLXNB3 Mutation Identified in Patients with Congenital Heart Disease with Neurodevelopmental Disabilities,” Online, Translational Pediatrics 11(11): 1852-1863; Bonthrone, A. F., Chew, A., Kelly, C. J., Almedom, L., Simpson, J., and 5 others 2021. “Cognitive Function in Toddlers with Congenital Heart Disease: The Impact of a Stimulating Home Environment,” Online, Infancy 26(1): 184-199; Kanazawa, H. and Fukuda, K. 2022. “The Plasticity of Cardiac Sympathetic Nerves and Its Clinical Implication in Cardiovascular Disease,” Online, Frontiers in Synaptic Science 14:960606; Leon, R, L., Mir, I. M., Herrera, C. I., Sharma, K., Spong, C. Y., Twickler, D. M., and Chalak, L, F. 2022. “Neuroplacentology in Congenital Heart Disease: Placental Connections to Neurodevelopmental Outcomes,” Online, Pediatric Research 91(4): 787-794; Dowden, L., Tucker, D., Morgan, S., Uzun, O., and Syed, Y, A. 2021. “Contribution of Congenital Heart Disorders Associated with Copy Number Variants in Mediating Risk for Brain Developmental Disorders: Evidence from 20-year Retrospective Cohort Study,” Online, Frontiers in Cardiovascular Medicine 8:655463; Asschenfeldt, B., Evald, L., Heiberg, J., Salvig, C., Ostergaard, L., and 3 others 2020. “Neuropsychological Status and Structural Brain Imaging in Adults with Simple Congenital Heart Defects Closed in Childhood,” Online, Journal of the American Heart Association (Oxford, England) 9(11): e015843; Ortinau, C. M. and Shimony, J. S. 2020. “The Congenital Heart Disease Brain: Prenatal Considerations for Perioperative Neurocritical Care,” Online, Pediatric Neurology 108:23-30; Klouda, L., Franklin, W. J., Saraf, A., Parekh, D. R., and Schwartz, D, D. 2017. “Neurocognitive and Executive Functioning in Adult Survivors of Congenital Heart Disease,” Congenital Heart Disease (Henderson, Nevada) 12(1): 91-98; Morton, P. D., Ishibashi, N., and Jonas, R, A. 2017. “Neurodevelopmental Abnormalities and Congenital Heart Disease: Insights into Altered Brain Maturation,” Online, Circulation Research 120(6): 960-977; Morton, P. D., Korotcova, L., Lewis, B. K., Bhuvanendran, S., Ramachandra, S. D., and 7 others 2017. “Abnormal Neurogenesis and Cortical Growth in Congenital Heart Disease,” Online, Science Translational Medicine 9(374): eaah7029; Cassidy, A. R., White, M. T., DeMaso, D. R., Newburger, J. W., and Bellinger, D, C. 2015. “Executive Function in Children and Adolescents with Critical Cyanotic Congenital Heart Disease,” Online, Journal of the International Neuropsychological Society 21(1): 34-49).

Compliance with medical guidance and adherence to a prescription if any represents a significant additional risk (see, for example, Fox, K. R., Vannatta, K., and Jackson J, L., 2023. “Difficulties with Executive Function are Associated with Risk Health Behaviors among Young Adult Congenital Heart Defect Survivors,” Journal of Cardiovascular Nursing 38(1): 60-69).

Above a threshold level of prenatal brain damage due to a sustained lack of normal heart function, hence, protracted hypoxia during postnatal development, neural stem or progenitor cells die or become too impaired to move out of the sellar region to build a normal cerebral cortex with normal dendritic arborization or to execute the repair of neural tissue as a factor that supports neural plasticity. Neural plasticity is critical for the recovery of cognitive function that allows a considerable measure of if not complete recovery after a stroke or undergoing a hemispherectomy to eradicate an intractable epilepsy, for example.

Circulation inadequacy during early development results in global maldevelopment, even such seemingly remote sequelae as maxillofacial malformities. Conventional surgical methods can achieve survival but not adequately repair a congenitally defective heart. Only replacement with a normal heart without the adverse consequences of surgical trauma, extended cardiopulmonary bypass, and general anesthesia can achieve normal development. Metered compound vascular heart transplantation is intended to accomplish this.

Furthermore, the trauma to both donor organ and the recipient in a conventional heart transplant substantially if not completely eliminated, the institution of circulatory normalcy even in infancy is insufficient to result in commendable performance in school. Constant apprehension over the possibility of rejection and the consequences of forgetting to adhere to the immunosuppressants prescribed are distractions that continue to contribute to poor performance in school. Responsive to these residual concerns, metered compound vascular heart transplantation eliminates trauma other than the painful entry wound and cooperates with the automatic disorder response system which administered the transplant to assure that patient adheres to the prescription regimen, eliminating this as a cause for anxiety.

The adverse neurocognitive, physiological, and structural implications of defective blood flow during early development continue to be elucidated (see for example, Chiperi, L. E., Tecar, C., and Hutanu, 2023. “A. Serum Tau Protein and Myelin Basic Protein in Pediatric Patients with Congenital Heart Defects Undergoing Cardiac Surgery: Preliminary Assessment as Novel Neuromarkers of Brain Injury,” Irish Journal of Medical Science (Dublin, Ireland) in print with abstract at Pubmed; Cromb, D., Bonthrone, A. F., Maggioni, A., Cawley, P., Dimitrova. R., and 12 others 2023. “Individual Assessment of Perioperative Brain Growth Trajectories in Infants with Congenital Heart Disease: Correlation with Clinical and Surgical Risk Factors,” Online, Journal of the American Heart Association (Oxford, England) 12(14): e028565; De Silvestro, A. A., Kellenberger, C. J., Gosteli. M., O'Gorman, R., and Knirsch, W. 2023. “Postnatal Cerebral Hemodynamics in Infants with Severe Congenital Heart Disease: A Scoping Review,” Pediatric Research (Basel, Switzerland) 94(3): 931-943; Easson, K., Khairy, M., Rohlicek, C. V., Gilbert, G., Majnemer, A., and 7 others 2023. “White Matter Microstructure is Differently Associated with Executive Functioning in Youth Bom with Congenital Heart Disease and Youth Bom Preterm,” Online, Brain and Behavior (Hoboken, New Jersey) 13(12): e3308; Naef, N., Hottinger, S., Schlosser, L., Greutmann, M., Latal, B., and O'Gorman, R. T. 2023. “Association of Cerebellar Volume with Cognitive and Motor Function in Adults with Congenital Heart Disease,” Online, Neurological Sciences (Milan, Italy) 44(11): 3979-3987; Bonthrone, A. F., Dimitrova, R., Chew. A., Kell.y C. J., Cordero-Grande, L., and 13 others 2021. “Individualized Brain Development and Cognitive Outcome in Infants with Congenital Heart Disease,” Online, Brain Communications (Oxford, England) 3(2): fcab046; Gatto, F., Perez-Rivas, L. G., Olarescu, N. C., Khandeva, P., Chachlaki, K., and 3 others 2020. “Diagnosis and Treatment of Parasellar Lesions,” Online, Neuroendocrinology (Basel, Switzerland) 110(9-10): 728-739; Simao, G. N. 2018. “Sellar and Parasellar Abnormalities,” Online, Radiologia Brasileira (Rio de Janeiro, Brazil) 51(1): I X; Pironkova, R. P., Giamelli, J., Seiden, H., Parnell, V. A., Gruber, D., and 3 others 2017. “Brain Injury with Systemic Inflammation in Newborns with Congenital Heart Disease Undergoing Heart Surgery,” Online, Experimental and Therapeutic Medicine (Athens, Greece) 14(1): 228-238; Dwyer, N. D., Chen, B. Chou, S., Hippenmeyer, S., Nguyen, L, and Ghashghaei, H. T. 2016. “Neural Stem Cells to Cerebral Cortex: Emerging Mechanisms Regulating Progenitor Behavior and Productivity,” Online, Journal of Neuroscience 36(45): 11394-11401; Latal, B. 2016. “Neurodevelopmental Outcomes of the Child with Congenital Heart Disease, Clinics in Perinatology (Philadelphia, Pennsylvania) 43(1): 173-85; Sathyanarayana, H. P., Kallasam, V., and. Chitharanjan, A, B. 2013. “Sella Turcica—Its Importance in Orthodontics and Craniofacial Morphology,” Online, Dental Research Journal (Mumbai [Bombay], India) 10(5): 571-575; Barbey, A. K., Colom, R., Solomon, J., Krueger, F., Forbes, C., and Grafman, J. 2012. “An Integrative Architecture for General Intelligence and Executive Function Revealed by Lesion Mapping,” Online, Brain (London, England) 135 (Pt 4): 1154-1164; Marino, B. S., Lipkin, P. H., Newburger, J. W., Peacock, G., Gerdes, M., and 9 others 2012. “Neurodevelopmental Outcomes in Children with Congenital Heart Disease: Evaluation and Management: A Scientific Statement from the American Heart Association,” Online, Circulation (Hagerstown, Maryland) 126(9): 1143-1172; Corbin, J. G., Gaiano, N., Juliano, S, L., Poluch, S., Stancik, E., and Haydar, T, F. 2008. “Regulation of Neural Progenitor Cell Development in the Nervous System,” Online, Journal of Neurochemistry 106(6): 2272-2287).

Normal neural plasticity requires normal neural stem cells—“Because NSCs are regulated by both intrinsic genetic and epigenetic programs and extrinsic stimuli transduced through the stem cell niche, dysregulation of NSCs due to either genetic causes or environmental impacts may lead to disease” (Zhao, X. and Moore, D. 2018. “Neural Stem Cells: Developmental Mechanisms and Disease Modeling,” Cell and Tissue Research 371(1): 1-6)(see also, for example, Kelly, C. J., Christiaens, D., Batalle, D., Makropoulos, A., Cordero-Grande, L., and 12 others 2019. “Abnormal Microstructural Development of the Cerebral Cortex in Neonates with Congenital Heart Disease is Associated with Impaired Cerebral Oxygen Delivery,” Journal of the American Heart Association (Oxford, England) 8(5): e009893; Leonetti, C., Back, S. A., Gallo, V., and Ishibashi, N. 2019. “Cortical Dysmaturation in Congenital Heart Disease,” Online, Trends in Neurosciences (Barking, East London, England) 42(3): 192-204; Kelly, C. J., Makropoulos, A., Cordero-Grande, L., Hutter, J., Price, A., and 15 others 2017. “Impaired Development of the Cerebral Cortex in Infants with Congenital Heart Disease is Correlated to Reduced Cerebral Oxygen Delivery,” (Nature Research) Scientific Reports 7(1): 15088; Morton, P. D., Korotcova, L., Lewis, B. K., Bhuvanendran, S., Ramachandra, S. D., and 7 others 2017, Op cit. above under (1). Congenital Defects of the Heart and Other Organs: The Importance of Prompt Correction; additional references below in this section).

Whether initiated postnatally by chronic hypoxia due to a congenitally maldeveloped heart or during the first trimester in gestation by a genetic defect in metabolism, neurocognitive deficiencies are probably the result of impairment in the ability to migrate or constitutional defects in neural stem cells (see, for example, Zhao, X. and Moore, D. 2018, Op cit.) or damage done to neural tissue after having been formed. “Although studies have suggested that irreversible nerve damage due to a high concentration of phenylalanine in the blood may be related to brain-derived neurotrophic factor, no studies have thoroughly examined the mechanism whereby hyperphenylalaninemia results in mental retardation” (Qi, Z., Cui, Y., Shi, L., Luan, J., Zhou, X., and Han, J. 2018. “Generation of Urine-derived Induced Pluripotent Stem Cells from a Patient with Phenylketonuria,” Intractable and Rare Diseases Research 7(2): 87-93).

Based upon the discontinuation of dietary therapy in phenylketonuria resulting in intellectual decline and the maternal phenylketonuria syndrome as including a high risk of heart malformity, phenylketonuria may cause mental retardation in two ways-damage to neural stem cells during embryonic development and damage to neurons due to the chronic hypoxia of abnormal circulation caused by a congenitally malformed heart (see, for example, Al Hafid, N. and Christodoulou, J. 2015. “Phenylketonuria: A Review of Current and Future Treatments,” Translational Pediatrics 4(4): 304-317).

Currently, heart transplantation can be accomplished following delivery, but not earlier when the normal development of the other organs, to include the brain, are most dependent upon a normal heart to provide the oxygen and nutrients needed to establish the other organs with the anatomy necessary for normal function. However, with the concurrent use of timing circuitry, positive or negative inotropes, and other medication as necessary, a central benefit in switch transplantation is that it makes possible heart transplantation in the fetus, and does so, moreover, through a method much less traumatizing and likely to provoke myocardial stunning, for example, than any conventional method of heart transplantation.

Iatrogenic injury to the brain in the effort to repair the defect or defects in the heart through conventional surgical means whereby the heart is opened can add to the impairment (see, for example, Claessens, N, H. P., Algra, S. O., Ouwehand, T, L., Jansen, N. J. G., Schappin, R., and 5 others 2018. “Perioperative Neonatal Brain Injury is Associated with Worse School-age Neurodevelopment in Children with Critical Congenital Heart Disease,” Developmental Medicine and Child Neurology 60(10): 1052-1058; Li, Y., Yin, S., Fang, J., Hua, Y., Wang, C., Mu, D., and Zhou, K. 2015. “Neurodevelopmental Delay with Critical Congenital Heart Disease is Mainly from Prenatal Injury Not Infant Cardiac Surgery: Current Evidence Based on a Meta-analysis of Functional Magnetic Resonance Imaging,” Ultrasound in Obstetrics and Gynecology 45(6): 639-648).

Because more serious cardiac defects in utero result postpartum in defective perfusion of the entire body, the intact or inadequately repaired body following conventional surgery is left to adapt to the abnormal circulation as best it can. The organism an integrated system, mechanical function is dependent upon metabolic performance and the reverse, so that health or the avoidance of repercussions in remote structures and tissues requires that every organ, gland, and tissue be healthy. The method for repairing d-transposition of the great arteries described herein is likewise extracardiac and minimally traumatizing, and therefore less likely to result in adverse consequences. Unless it is removed and replaced with a healthy heart, to be bom with a sick of substandard heart is to be born with a sick body—to be sick altogether, and the irreversibility of this sickness from the outset will remain with the patient to the end of life.

Everything organ dependent upon every other, any organ that fails to perform normally and resists correcton through less extreme measures must simply be cut out and replaced-promptly, without inadequate measures and without delay. This is true in a patient of any age; when later in life the heart, liver, kidneys, lungs, or a gland become diseased or otherwise impaired, the interruption in normal physiology will soon become evident in the malfunction of the other organs. The practicability of such an approach materialized only with the appearance of immunosuppressants and somatic support systems.

Currently, repair of a prenatal heart consists of ultrasound-guided transcatheterie or transluminal repair of aortic stenosis and valvuloplasty, atrial septoplasty; pulmonary atresia, and an emerging hypoplastic left heart syndrome with a restrictive interatrial septum (Freud L, R. and Tworetzky, W. 2016. Op cit. in the preceding section). Other congenital defects can also be repaired in utero through transuterine or cesarean/extrauterine repair and minimally invasive fetoscopic surgery. Corrective surgery is less complex and risky if performed following delivery. However, mechanical support in the form of ventricular assist devices or a total artificial heart continues to be eluded by the tiny size of the patient, and would only serve as a bridging strategy pending availability of a replacement heart in any event, a requirement the pediatric cardiac surgeon can usually satisfy.

Completely extracardiac, switch technique eliminates the need for cardiopulmonary machine support, aortic cross-clamping, cardioplegia, and general rather than regional anesthesia, considerably reducing the risks involved and making the procedure practicable for the larger number of thoracic surgeons. The level of skill required to perform a heart transplant involving incision into the heart is greater. The shortage of replacement organs the most pressing challenge to organ transplantation, this problem is considerably greater in the procurement of hearts for placement in neonates where the benefit would be greatest. A method for replacing the defective heart, which unlike the bicaval and biatrial techniques that require considerable incision into the heart, leaves the heart completely intact, should not result in a decline in left ventricular perfusion or contractile function following transplant (references cited above in this section).

While endocrine regulation over heart rate and contractility mediated through the bloodstream should be little affected, heart transplantation using a switch technique involves cutting the autonomic nerve fibers that convey the extrinsic cardiac innervation no differently than in a biatrial or bicaval transplantation. The extrinsic innervation of the heart is complex and understood (see, for example, Adjei. T., von Rosenberg, W., Nakamura. T., Chanwimalueang. T., and Mandic, D. P. 2019. “The ClassA [Classification Angle] Framework: HRV [heart rate variability] Based Assessment of SNS [sympathetic nervous system] and PNS [parasympathetic nervous system] Dynamics without LF-HF [low frequency and high frequency] Controversies,” Online, Frontiers in Physiology 10:505: Gurel, N. Z., Carek, A. M., Inan, O. T., Levantsevych, O., Abdelhadi. N., and 15 others 2019. “Comparison of Autonomic Stress Reactivity in Young Healthy versus Aging Subjects with Heart Disease,” Online, PLoS [Public Library of Science] One 14(5): e0216278; Giuliano, R. J., Gatzke-Kopp. L. M., Roos. L. E., and Skowron, E, A. 2017. “Resting Sympathetic Arousal Moderates the Association between Parasympathetic Reactivity and Working Memory Performance in Adults Reporting High Levels of Life Stress,” Psychophysiology 54(8): 1195-1208; Beauchaine. T. P., Bell, Z., Knapton, E., McDonough-Caplan, H., Shader, T., and Zisner, A. 2019. “Respiratory Sinus Arrhythmia Reactivity across Empirically Based Structural Dimensions of Psychopathology: A Meta-analysis,” Psychophysiology 56(5): e13329; Gordan, R., Gwathmey, J. K., and Xie. L, H. 2015. “Autonomie and Endocrine Control of Cardiovascular Function,” World Journal of Cardiology 7(4): 204-214). Nervous disconnection does not protect the transplanted heart from the adverse effects of great stress, which are mediated by hormones.

Unpredictable, the consequences of early postoperative denervation are well doumented based upon the empirical evidence and do not rise to a level of adversity or severity that would have dissuaded transplantation. The postprocedural loss of extrinsic cardiac innervation spontaneously recovers, but not completely (see, for example, Awad, M., Czer, L. S., Hou, M., Golshani. S. S., Goltche, M., and 8 others 2016. “Early Denervation and Later Reinnervation of the Heart following Cardiac Transplantation: A Review,” Journal of the American Heart Association (Oxford, England) 5(11). pii: e004070; Lee, S. R., Kang, D. Y., Cho, Y., Cho, H. J., Lee, H. Y., Choi, E. K., and Oh, S. 2016. “Early Parasympathetic Reinnervation is Not Related to Reconnection of Major Branches of the Vagus Nerve after Heart Transplantation,” Korean Circulation Journal 46(2): 197-206; Gallego-Page, J. C., Segovia, J., Alonso-Pulpon, L., Alonso-Rodriguez, M., Salas. C., and Ortiz-Berrocal, J. 2004. “Re-innervation after Heart Transplantation: A Multidisciplinary Study,” Journal of Heart and Lung Transplantation (New York, New York) 23(6): 674-682).

Given spontaneous recovery to at least a partial extent, nerve allografts supported by an immunosuppressant with or without pluripotent stem cells directly targeted to the blood supply of the neural anastomosis through a side-entry jacket by the implant disorder diagnostic and response system to support a more thorough recovery of autonomic control should seldom prove necessary (see, for example, Elkwood, A. I., Holland, N. R., Arbes, S. M., Rose, M. I., Kaufman, M. R., and 3 others 2011. “Nerve Allograft Transplantation for Functional Restoration of the Upper Extremity: Case Series,” Journal of Spinal Cord Medicine 34(2): 241-247; Pendleton, C., Ahmed, I., and Quinones-Hinojosa, A. 2011. “Neurotransplantation: Lux et Veritas [light and truth]. Fiction or Reality?,” Journal of Neurosurgical Sciences 55(4): 297-304).

A central advantage in the means to be described is the allowance of different strategies to allow an expansion in the zone and therefore the pool of acceptable replacement hearts for the very young. It having been found that a replacement heart over- or undersized within limits for a tiny patient will gradually adapt to the reduction in load (see, for example, Shin, H. J., Jhang, W. K., Park, J. J., Yun, T. J., Kim, Y. H., and 3 others 2011. “Heart Transplantation in Pediatric Patients: Twelve-year Experience of the Asan Medical Center,” Journal of Korean Medical Science 26(5): 593-598; Kertesz, N. J., Gajarski, R. J., Towbin, J. A., and Geva. T. 1995. “Effect of Donor-recipient Size Mismatch on Left Ventricular Remodeling after Pediatric Orthotopic Heart Transplantation,” American Journal of Cardiology 76(16): 1167-1172; see also section 2b(1)(b)(b2), entitled Prenatal Compound Vascular Bypass, or Sudden Switched-organ Transplantation of the Heart), this in itself makes more hearts available.

Intentional denervation has been proposed as a treatment modality for alleviating arrhythmia (Witt, C. M., Bolona, L., Kinney, M. O., Moir. C., Ackerman, M. J., and 3 others 2017. “Denervation of the Extrinsic Cardiac Sympathetic Nervous System as a Treatment Modality for Arrhythmia,” Europace 19(7): 1075-1083; Irie, T., Yamakawa, K., Hamon, D., Nakamura, K., Shivkumar, K., and Vaseghi. M. 2017. “Cardiac Sympathetic Innervation via Middle Cervical and Stellate Ganglia and Antiarrhythmic Mechanism of Bilateral Stellectomy,” American Journal of Physiology. Heart and Circulatory Physiology 312(3): H392-H405; Kosiuk, J., Hilbert, S., Pokushalov, E., Hindricks, G., Steinberg, J. S., and Bollmann, A. 2015. “Renal Denervation for Treatment of Cardiac Arrhythmias: State of the Art and Future Directions,” Journal of Cardiovascular Electrophysiology 26(2): 233-238).

The problem with an oversized heart is the initial hypertension, which can be life-threatening. With the patient constantly, equipped with sensors to detect hypertension and instantly release counteractants, averting further consequences, a fully implanted prosthetic disorder control system best serves to allow extension in the supply of replacement hearts to those of somewhat older children. The system shepherds the process of continuously transitioning blood flow through the graft organ and donor of recipient blood, signaling the need to slow down the process.

It then remains implanted postprocedurally to automatically release the immunosuppressant, respond to noncritical detection of inflammation through the automatic targeting of medication to the treatment site, and if equipped with telemetry, can immediately alert the clinic to a crisis condition. Increase in the availability of replacement hearts is the first factor to advance the cause of heart repair in the very young. The other factor is avoidance of the need to enter the heart, made possible by use of the sudden or the metered switch technique. A heart transplant is still unavoidably open-chest and full of risk, but these two measures materially reduce the risk.

Adaptation to a malformed heart typically includes the development of collateral circulation, almost always inadequate, in some instances, posing the risk of rupture, and vascular remodeling, which is limited (see, for example, Dipchand, A. I. 2018, Op cit. above in section 2a; Kenny, L. A., DeRita, F., Nassar, M., Dark, J., Coats, L., and Hasan, A. 2018. “Transplantation in the Single Ventricle Population,” Annals of Cardiothoracic Surgery (Hong Kong, China) 7(1): 152-159; Das, B. B. 2018. Op cit. above under (a). Sudden compound vascular bypass, or sudden switched- organ transplantation; van der Ven, J. P. G., van den Bosch, E., Bogers, A. J, C. C., and Helbing, W, A. 2018. “State of the Art of the Fontan Strategy for Treatment of Univentricular Heart Disease,” Faculty of 1000 Research 7. pii: F1000 Faculty Rev-935; Kverneland, L. S., Kramer, P., and Ovroutski, S. 2018. “Five Decades of the Fontan operation: A Systematic Review of International Reports on Outcomes after Univentricular Palliation,” Congenital Heart Disease 13(2): 181-193; Hauck, A., Porta, N., Lestrud. S., and Berger, S. 2017. “The Pulmonary Circulation in the Single Ventricle Patient,” Children (Basel, Switzerland) 4(8). pii: E71; Hinton, R, B. and Ware, S. M. 2017. “Heart Failure in Pediatric Patients with Congenital Heart Disease,” Circulation Research 120(6): 978-994; Ohuchi, H. 2017. “Where is the “Optimal” Fontan Hemodynamics?.” Korean Circulation Journal 47(6): 842-857; François, K., Vandekerckhove, K., De Groote, K., Panzer, J., De Wolf. D., De Wilde, H., and Bove. T. 2016. “Current Outcomes of the Bi-directional Cavopulmonary Anastomosis in Single Ventricle Patients: Analysis of Risk Factors for Morbidity and Mortality, and Suitability for Fontan Completion,” Cardiology in the Young (Cambridge, England) 26(2): 288-297; Li, F., Cai, J., Sun, Y. F., Liu, J. P., and Dong, N, G. 2015. “Pediatric Heart Transplantation: Report from a Single Center in China,” Chinese Medical Journal (English edition) 128(17): 2290-2294; Lluri. G., Levi, D. S., and Aboulhosn. J. 2015. “Systemic to Pulmonary Venous Collaterals in Adults with Single Ventricle Physiology after Cavopulmonary Palliation,” International Journal of Cardiology 189:159-163; Huebler, M., Schubert, S., Lehmkuhl, H. B., Weng. Y., Miera, O., and 3 others 2011. “Pediatric Heart Transplantation: 23-year Single-center Experience,” European Journal of Cardiothoracic Surgery 39(5): e83-e89; Heinemann, M., Breuer, J., Steger. V., Steil, E., Sieverding, L., and Ziemer, G. 2001. “Incidence and Impact of Systemic Venous Collateral Development after Glenn and Fontan Procedures,” Thoracic and Cardiovascular Surgery 49(3): 172-178). Weber, H. S. 2001. “Incidence and Predictors for the Development of Significant Supradiaphragmatic Decompressing Venous Collateral Channels following Creation of Fontan Physiology,” Cardiology in the Young (Cambridge, England) 11(3): 289-294; additional references cited in relevant sections to follow).

In this, the advantage of prompt replacement in a fetus or neonate of a heart transplant before secondary consequences of faulty circulation arise, preferably with the sudden or metered switch methods set forth herein, is clear: the source of multiorgan sequelae of abnormal circulation will have been preempted. Sudden and metered switch heart transplantation in neonates and adults is distinguished from the foregoing in immunological rather than mechanical terms.

So long as the abnormal circulation continues, the chronic hypoxia leads to global secondary deficits in all organs and tissues, can affect the kidneys, leading to the disrupted production of blood electrolytes, and maldevelopment of the endocrine glands, leading to hormonal shortcomings, for example, most secondary defects arising along the digestive tract and in the brain. Since all the organs affect the blood, multiorgan degeneration ensues when a functionally defective liver or kidneys are operated on rather than replaced, the impulse favoring preservation rather than “trading one disease for another” due to the lifelong need for immunosuppressants even though these will continue to improve. What is true of the heart is no less true of the other organs; the body functions as an integral unit in which every organ, gland, and tissue contributes an essential part, so that the dysfunction of any adversely affects the rest.

The body an integral unit, while impaired circulation from before birth leads to degradation in all organs, glands, and tissues, making the prompt replacement of a heart so malformed that it cannot be restored to nomal circulation critical, the relation of the heart to the rest of the body one of reciprocal dependency. To stop progressive multiple organ damage, which includes maldevelopment of the brain, lungs, kidneys, and liver, transplantation should be undertaken before birth, and preferably, as soon as the heart can be discerned to deviate from normal development-most often at three to four months postconception, currently, primarily by means of nuchal translucency and Doppler echocardiography screening.

At this stage in development, the vessels and the valves used to accomplish a switch method heart transplant, which best march the vessels in caliber, are tiny so that to operate with these necessitates high magnification (see, for example, Qasim, A., Johnson, C. B., Aly, M. A., and Aly, A. M. 2019. “Prenatal Diagnosis and Successful Palliation of Absent Aortic Valve with Hypoplastic Left Heart Syndrome: A Case Report and Review of Literature,” American Journal of Perinatology Reports 9(2): e121-e126; Yeo, L., Markush, D., and Romero, R. 2019. “Prenatal Diagnosis of Tetralogy of Fallot with Pulmonary Atresia Using: Fetal Intelligent Navigation Echocardiography (FINE),” Journal of Maternal-Fetal and Neonatal Medicine 32(21): 3699-3702; Bravo-Valenzuela, N. J., Peixoto, A. B., and Araujo Junior, E. 2018. “Prenatal Diagnosis of Congenital Heart Disease: A Review of Current Knowledge,” Indian Heart Journal 70(1): 150-164; Yeo, L., Luewan, S., and Romero, R. 2018. “Fetal Intelligent Navigation Echocardiography (FINE) Detects 98% of Congenital Heart Disease,” Journal of Ultrasound in Medicine 37(11): 2577-2593; Bronshtein, M., Blumenfeld, Z., Khoury, A., and Gover, A. 2017. “Diverse Outcome following Early Prenatal Diagnosis of Pulmonary Stenosis,” Ultrasound in Obstetrics and Gynecology 49(2): 213-218; Chitra, N. and Vijayalakshmi. I, B. 2017. “Fetal Echocardiography for Early Detection of Congenital Heart Diseases,” Journal of Echocardiography 15(1): 13-17; Yeo. L. and Romero, R. 2017. “Prenatal Diagnosis of Hypoplastic Left Heart and Coarctation of the Aorta with color Doppler FINE [Fetal Intelligent Navigation Echocardiography],” Ultrasound in Obstetrics and Gynecology 50(4): 543-544; Yu. R., Li, S, L., Yeo. L. and Romero, R. 2017. “Color and Power Doppler Combined with Fetal Intelligent Navigation Echocardiography (FINE) to Evaluate the Fetal Heart,” Ultrasound in Obstetrics and Gynecology 50(4): 476-491; Luo, G. Y., Wen, H. X., Ouyang, S. Y., Chen, C. Y., and Yuan, Y. 2016. “First-trimester Echocardiographic Features and Perinatal Outcomes in Fetuses with Congenital Absence of the Aortic Valve,” Journal of Ultrasound in Medicine 35(4): 739-745; Yeo. L. and Romero, R. 2016. “Intelligent Navigation to Improve Obstetrical Sonography,” Ultrasound in Obstetrics and Gynecology 47(4): 403-409; Liu, H., Zhou, J., Feng, Q, L., Gu, H. T., Wan, G., and 3 others 2015. “Fetal Echocardiography for Congenital Heart Disease Diagnosis: A Meta-analysis, Power Analysis and Missing Data Analysis,” European Journal of Preventive Cardiology 22(12): 1531-1547; Turan, S., Turan, O. M., Desai, A., Harman, C. R., and Baschat, A. A. 2014. “First-trimester Fetal Cardiac Examination Using Spatiotemporal Image Correlation, Tomographic Ultrasound and Color Doppler Imaging for the Diagnosis of Complex Congenital Heart Disease in High-risk Patients,” Ultrasound in Obstetrics and Gynecology 44(5): 562-567; Eleftheriades, M., Tsapakis. E., Sotiriadis, A., Manolakos, E., Hassiakos, D., and Botsis, D. 2012. “Detection of Congenital Heart Defects throughout Pregnancy; Impact of First Trimester Ultrasound Screening for Cardiac Abnormalities,” Journal of Maternal-Fetal and Neonatal Medicine 25(12): 2546-2550; additional references cited below in section 2a(2), entitled Inventive Content-pertinent Fetal Diagnostics.

Genetic and biochemical diagnostics afford another means for detecting whether cardiac abnormalities are present and offers the prospect of therapeutic intervention in adult atherosclerosis and heart failure, as well as to avert continued cardiac abnormalities in utero and so eliminate the need for postpartum surgery either corrective or by means of heart transplantation (see, for example, Duan, H, L., Zhu, X. Y., Zhu, Y. J., Wu. X., Zhao, G. F., Wang, W. J., and Li, J. 2019. “The Application of Chromosomal Microarray Analysis to the Prenatal Diagnosis Isolated Mild Ventriculomegaly,” Taiwan Journal of Obstetrics and Gynecology 58(2): 251-254; Li, M., Duan, L., Li, Y., and Liu, B. 2019. “Long Noncoding RNA/Circular Noncoding RNA-miRNA-mRNA Axes in Cardiovascular Diseases,” Life Sciences April 29 pii: S0024-3205(19) 30334-0; Wang. W., Wang. Y., Piao. H., Li, B., Huang. M., and 5 others 2019. “Circular RNAs as Potential Biomarkers and Therapeutics for Cardiovascular Disease,” PeerJ 7: e6831; Bayoumi, A. S., Aonuma, T., Teoh, J. P., Tang, Y, L., and Kim, I. M. 2018. “Circular Noncoding RNAs as Potential Therapies and Circulating Biomarkers for Cardiovascular Diseases,” Acta Pharmacologica Sinica 39(7): 1100-1109; Cai. M., Huang, H., Su, L., Lin, N., Wu X., and 5 others 2018. “Fetal Congenital Heart Disease: Associated Anomalies, Identification of Genetic Anomalies by Single-nucleotide Polymorphism Array Analysis, and Postnatal Outcome,” Medicine (Baltimore, Maryland) 97(50): e13617; E. S., Costa, M. C., Kurc. S., Drożdż, A., Cortez-Dias, N., and Enguita, F. J. 2018. “The Circulating Non-coding RNA Landscape for Biomarker Research: Lessons and Prospects from Cardiovascular Diseases,” Acta Pharmacolica Sinica 39(7): 1085-1099; He, L., Chen, Y., Hao. S., and Qian, J. 2018. “Uncovering Novel Landscape of Cardiovascular Diseases and Therapeutic Targets for Cardioprotection via Long Noncoding RNA-miRNA-mRNA Axes,” Epigenomics 10(5): 661-671; Huang, Y. 2018. “The Novel Regulatory Role of lncRNA-miRNA-mRNA Axis in Cardiovascular Diseases,” Journal of Cellular and Molecular Medicine 22(12): 5768-5775; Saxena, S., Gupta, A., Shukla, V., and Rani, V. 2018. “Functional Annotation of Differentially Expressed Fetal Cardiac microRNA Targets: Implication for microRNA-based Cardiovascular Therapeutics,” 3 Biotech 8(12): 494; Wang. Y., Hu. P., and Xu. Z. 2018. “Copy Number Variations and Fetal Ventriculomegaly,” Current Opinion in Obstetrics and Gynecology 30(2): 104-110; Xia, Y., Yang, Y., Huang, S., Wu. Y., Li, P., and Zhuang, J. 2018. “Clinical Application of Chromosomal Microarray Analysis for the Prenatal Diagnosis of Chromosomal Abnormalities and Copy Number Variations in Fetuses with Congenital Heart Disease,” Prenatal Diagnosis 38(6): 406-413; Gao. J., Xu. W., Wang. J., Wang, K., and Li, P. 2017. “The Role and Molecular Mechanism of Non-coding RNAs in Pathological Cardiac Remodeling,” Online, MDPI [Multidisciplinary Digital Publishing Institute] International Journal of Molecular Sciences (Basel, Switzerland) 18(3). pii: E608; Gu. H., Liu, Z., and Zhou, L. 2017. “Roles of miR-17-92 Cluster in Cardiovascular Development and Common Diseases,” Online, BioMed Research International 2017:9102909; Liu, Y., Xie, J., Geng, Q., Xu, Z., Wu. W., and 6 others 2017. “Combined G-banded Karyotyping and Multiplex Ligation-dependent Probe Amplification for the Detection of Chromosomal Abnormalities in Fetuses with Congenital Heart Defects,” (in Chinese with English abstract at Pubmed), Zhonghua Yi Xue Yi Chuan Xue Za Zhi [Chinese Journal of Medical Genetics] 34(1): 1-5; Oneda, B. and Rauch, A. 2017. “Microarrays in Prenatal Diagnosis,” Best Practice and Research. Clinical Obstetrics and Gynaecology 42:53-63; Wojciechowska, A., Braniewska, A., and Kozar-Kaminska, K. 2017. “MicroRNA in Cardiovascular Biology and Disease,” Advances in Clinical and Experimental Medicine 26(5): 865-874; Ai. F., Zhang, Y., and Peng, B. 2016. “miR-20a Regulates Proliferation, Differentiation and Apoptosis in P19 Cell Model of Cardiac Differentiation by Targeting Smoothened” Biology Open 5(9): 1260-1265; Edwards, J. J. and Gelb, B, D. 2016. “Genetics of Congenital Heart Disease,” Current Opinion in Cardiology 31(3): 235-241; additional references cited below under 2. Background of the Invention, a. General, (2). Fetal Diagnostics).

Attempts at repair rather than replacement of a severely malformed heart even by the best pediatric cardiac surgeons, perfusionists, and anesthesiologists are currently instituted after developmental deficits have already formed and the procedure can itself cause injury to the brain, can do so directly, indirectly, or both, making prenatal transplantation preferable when a replacement heart is available. Availability is a major object in the use of an implanted automatic response system and double heart metered switch heart transplantation to justify the use of marginal hearts that would otherwise be rejected for transplantation.

Brain injury can even occur as a sequela to less intricate surgery related or unrelated to the heart and can be due to the surgery itself, the support equipment, its use, or a combination of these (see, for example, Greaves, D., Psaltis, P. J., Ross, T. J., Davis, D., Smith, A. E., Boord, M. S., and Keage, H, A, D. 2019. “Cognitive Outcomes following Coronary Artery Bypass Grafting: A Systematic Review and Meta-analysis of 91.829 Patients,” International Journal of Cardiology 289:43-49; Safavynia, S, A. and Goldstein, P, A. 2019. “The Role of Neuroinflammation in Postoperative Cognitive Dysfunction: Moving from Hypothesis to Treatment,” Online, Frontiers in Psychiatry 9:752; Thudium, M., Ellerkmann, R. K., Heinze, I., and Hilbert, T. 2019. “Relative Cerebral Hyperperfusion during Cardiopulmonary Bypass is Associated with Risk for Postoperative Delirium: A Cross-sectional Cohort Study,” Bio Med Central Anesthesiology 19(1): 35; Nagre, A. S. 2018. “Perioperative Stroke-Prediction, Prevention, and Protection,” Indian Journal of Anaesthesia 62(10): 738-742; Nijs, K., Vandenbrande, J., Vaqueriza, F., Ory, J. P., Yilmaz, A., and 5 others 2017. “Neurological Outcome after Minimal Invasive Coronary Artery Surgery (NOMICS): Protocol for an Observational Prospective Cohort Study,” Online, British Medical Journal Open 7(10): e017823; Zou, R., Shi, W., Tao, J., Li, H., Lin, X., Yang, S., and Hua, P. 2017. “Neurocardiology: Cardiovascular Changes and Specific Brain Region Infarcts,” Online, BioMed Research International 2017:5646348; Scolletta, S., Taccone, F. S., and Donadello, K. 2015. “Brain Injury after Cardiac Surgery,” Minerva Anestesiologica 81(6): 662-677; Goto, T. and Maekawa, K. 2014. “Cerebral Dysfunction after Coronary Artery Bypass Surgery,” Journal of Anesthesia 28(2): 242-248; Nemeth, A., Hejjel, L., Ajtay, Z., Kellenyi, L., Solymos. A., and 5 others 2013. “The Assessment of Neural Injury following Open Heart Surgery by Physiological Tremor Analysis,” Archives of Medical Science 9(1): 40-46; Mehta. Y. and Singh, R. 2010. “Cognitive Dysfunction after Cardiac Surgery,” Journal of Alzheimer's Disease 22 Supplement 3:115-120; Gottesman, R. F., Mckhann, G. M., and Hogue, C. W. 2008. “Neurological Complications of Cardiac Surgery,” Seminars in Neurology 28(5): 703-715; Grogan, K., Stearns, J., and Hogue, C. W. 2008. “Brain Protection in Cardiac Surgery,” Anesthesiology Clinics 26(3): 521-538; Sulejczak, D., Grieb. P., Walski. M., and Frontczak-Baniewicz, M. 2008. “Apoptotic Death of Cortical Neurons following Surgical Brain Injury,” Folia Neuropathologiea 46(3): 213-219; Lund, C., Sundet, K., Tennoe, B., Hol, P. K., Rein, K. A., Fosse, E., and Russell, D. 2005. “Cerebral Ischemic Injury and Cognitive Impairment after Off-pump and On-pump Coronary Artery Bypass Grafting Surgery,” Annals of Thoracic Surgery 80(6): 2126-2131; Teixeira-Sousa, V., Costa, C., Costa, A., Grangeia, R., Reis. C., and Coelho, R. 2008. “Neurocognitive Dysfunction after Valve Surgery,” (in Portuguese with English abstract at Pubmed), Acta Medica Portuguesa 21(5): 475-482; Engelman, R. 2001. “The Neurologic Complications of Cardiac Surgery: Introduction,” Seminars in Thoracic and Cardiovascular Surgery 13(2): 147-148; Barbut, D. and Caplan, L, R. 1997. “Brain Complications of Cardiac Surgery,” Current Problems in Cardiology 22(9): 449-480; Furlan, A. J., Sila, C. A., Chimowitz, M. I., and Jones, S, C. 1992. “Neurologic Complications Related to Cardiac Surgery,” Neurological Clinics 10(1): 145-166; Sotaniemi, K, A. 1980. “Brain Damage and Neurological Outcome after Open-heart Surgery,” Journal of Neurology. Neurosurgery, and Psychiatry 43(2): 127-135).

Switch heart transplantation is thus safer than attempts at a difficult repair, and the results likely to be better. The immediate and targeted support of a transplant heart by an implanted response system as described in U.S. Pat. No. 11,759,186, Ductus Side-entry Jackets and Prosthetic Disorder Response Systems, makes the placement of a marginal heart or one considered oversized safer expanding the zone of available hearts even for a conventional orthotopic transplantation. Eliminating ischemia, cardiopulmonary bypass, cardioplegia, cross-clamping, and general anesthesia, switch transplantation further expands the zone by eliminating almost all of the trauma imposed on the donor heart. The trauma is reduced to that associated with removal from the donor.

The body as a whole and each organ are interdependent so that the impairment of any one organ or gland leads to impairment of the others, this interdependency (Anderson, W. D., DeCicco, D., Schwaber, J. S., and Vadigepalli, R. 2017. “A Data-driven Modeling Approach to Identify Disease-specific Multi-organ Networks Driving Physiological Dysregulation” Online, PLoS [Public Library of Science] Computational Biology 13(7): e1005627) progressively making the baby sicker and sicker. Accordingly, conventional transplantation of the lungs, one or both kidneys, or the liver, for example, with the recipient placed on life support, can reciprocally lead to multiorgan and gland impairment, and Fontan repair with its nonpulsatile passive flow following univentricular palliation affects the development of:

a. The brain, previously addressed in the preceding section, wherein impairment ensues with, much less without, surgical intervention if the infant survives. Genetic mutations that affect both the heart and brain remaining beyond the current capability to correct, here the concern is heart malformity before or after the attempt at repair as the causative factor for abnormal circulation and neurocognitive impairmament (see, for example, Claessens, N, H. P., Breur, J. M. P. J., Groenendaal, F., Wosten-van Asperen, R. M., Stegeman, R., and 5 others 2019. “Brain Microstructural Development in Neonates with Critical Congenital Heart Disease: An Atlas-based Diffusion Tensor Imaging Study,” Neuroimage. Clinical 21: 101672; Kelly, C. J., Christiaens. D., Batalle, D., Makropoulos, A., Cordero-Grande, L., and 12 others 2019. Op cit. above in this section; Leonetti, C., Back, S. A., Gallo, V., and Ishibashi, N. 2019 Op cit. above under (1). Congenital Defects of the Heart and Other Organs: the Importance of Prompt Correction; Melazzini, L., Codari, M., Vitali, P., and Sardanelli, F. 2019. “Brain Vascular Changes in Adults with Congenital Heart Disease: A Systematic Review,” NeuroImage. Clinical 23:101873; Peyvandi, S., Latal, B., Miller, S. P., and McQuillen, P. S. 2019. Op cit., this section; Guo, T., Chau. V., Peyvandi, S., Latal. B., McQuillen. P. S., and 9 others 2019. “White Matter Injury in Term Neonates with Congenital Heart Diseases: Topology and Comparison with Preterm Newborns,” Neuroimage 185:742-749; Robson, V. K., Stopp, C., Wypij, D., Dunbar-Masterson, C., Bellinger, D. C., and 3 others 2019. “Longitudinal Associations between Neurodevelopment and Psychosocial Health Status in Patients with Repaired D-transposition of the Great Arteries,” Journal of Pediatrics 204:38-45.e1; Chatzis, A, C. and Grisbolaki, E. 2018. “Preoperative Detrimental Effect on Cerebral Function of Severe Congenital Heart Disease,” Journal of Thoracic Disease 10 (Supplement 33): S3973-S3975; Codari, M., Papini, G, D. E., Melazzini, L., Pluchinotta, F. R., Secchi, F., and 4 others 2018. “Does Tetralogy of Fallot Affect Brain Aging? A Proof-of-concept Study,” Online, PLoS [Public Library of Science] One 13(8): e0202496; Zaidi, A. H., Newburger, J. W., Wypij. D., Stopp. C., Watson, C. G., and 3 others 2018. “Ascending Aorta Size at Birth Predicts White Matter Microstructure in Adolescents who Underwent Fontan Palliation,” Journal of the American Heart Association (Oxford, England) 7(24): e010395; Schmithorst, V. J., Votava-Smith, J. K., Tran, N., Kim, R., Lee. V., and 7 others 2018. “Structural Network Topology Correlates of Microstructural Brain Dysmaturation in Term Infants with Congenital Heart Disease,” Human Brain Mapping 39(11): 4593-4610; Claessens, N, H. P., Algra, S. O., Ouwehand, T, L., Jansen, N. J. G., Schappin, R., and 5 others 2018; Op cit. above in this section; Karmacharya, S., Gagoski, B., Ning, L., Vyas. R., Cheng, H. H., and 5 others 2018. “Advanced Diffusion Imaging for Assessing Normal White Matter Development in Neonates and Characterizing Aberrant Development in Congenital Heart Disease,” Neuroimage. Clinical 19:360-373; Lynch, J. M., Ko. T., Busch, D. R., Newland, J. J., Winters, M. E., and 10 others 2018. “Preoperative Cerebral Hemodynamics from Birth to Surgery in Neonates with Critical Congenital Heart Disease,” Journal of Thoracic and Cardiovascular Surgery 156(4): 1657-1664; van der Ven, J. P. G., van den Bosch, E., Bogers, A. J, C. C., and Helbing, W, A. 2018. “State of the Art of the Fontan Strategy for Treatment of Univentricular Heart Disease,” F1000 Research 27:7. pii: F1000 Faculty Rev-935; Mebius, M. J., Kooi, E. M. W., Bilardo, C. M., and Bos, A, F. 2017. “Brain Injury and Neurodevelopmental Outcome in Congenital Heart Disease: A Systematic Review,” Pediatrics 140(1). pii: e20164055; Morton, P. D., Korotcova, L., Lewis, B. K., Bhuvanendran, S., Ramachandra, S. D., and 7 others 2017, Op cit. above under (1). Congenital Defects of the Heart and Other Organs: The Importance of Prompt Correction; Calderon, J., Willaime, M., Lelong, N., Bonnet, D., Houyel, L., and 3 others 2017. “Population-based Study of Cognitive Outcomes in Congenital Heart Defects,” Archives of Disease in Childhood August 5. pii: archdischild-2016-310830; DeMaso, D. R., Calderon, J., Taylor, G. A., Holland, J. E., Stopp. C., and 5 others 2017. “Psychiatric Disorders in Adolescents with Single Ventricle Congenital Heart Disease,” Pediatrics 139(3). pii: e20162241; Kelly, C. J., Makropoulos. A., Cordero-Grande, L., Hutter, J., Price, A., and 15 others 2017, Op cit., this section; Pironkova, R. P., Giamelli, J., Seiden, H., Parnell, V. A., Gruber, D., and 3 others 2017. “Brain Injury with Systemic Inflammation in Newborns with Congenital Heart Disease Undergoing Heart Surgery,” Experimental and Therapeutic Medicine 14(1): 228-238; Man, T., He, Y., Zhao, Y., Sun, L., Liu, X., and Ge. S. 2017. “Cerebrovascular Hemodynamics in Fetuses with Congenital Heart Disease,” Echocardiography 34(12): 1867-1871; Lim, J. M., Kingdom, T., Saini, B., Chau, V., Post. M., and 4 others 2016. “Cerebral Oxygen Delivery is Reduced in Newborns with Congenital Heart Disease,” Journal of Thoracic and Cardiovascular Surgery 152(4): 1095-1103; Jansen, F. A., van Zwet, E. W., Rijlaarsdam, M. E., Pajkrt E., van Velzen, C, L., and 7 others 2016, Op cit. above under 1. Background of the Invention. a. General; Marelli, A., Miller, S. P., Marino, B. S., Jefferson, A, L., and Newburger, J. W. 2016. “Brain in Congenital Heart Disease across the Lifespan: The Cumulative Burden of Injury,” Circulation 133(20): 1951-1962; Khalil. A., Bennet, S., Thilaganathan, B., Paladini, D., Griffiths, P., and Carvalho, J. S. 2016. “Prevalence of Prenatal Brain Abnormalities in Fetuses with Congenital Heart Disease: A Systematic Review,” Ultrasound in Obstetrics and Gynecology 48(3): 296-307; Calderon, J., Stopp. C., Wypij. D., DeMaso, D. R., Rivkin, M., Newburger, J. W., and Bellinger, D, C. 2016. “Early-term Birth in Single-ventricle Congenital Heart Disease after the Fontan Procedure: Neurodevelopmental and Psychiatric Outcomes,” Journal of Pediatrics 179:96-103; Hovels-Gurich, H, H. 2016. “Factors Influencing Neurodevelopment after Cardiac Surgery during Infancy,” Online, Frontiers in Pediatrics 4:137; Knirsch, W., Liamlahi, R., Dave, H., Kretschmar, O., and Latal, B. 2016. “Neurodevelopmental Outcome of Children with Hypoplastic Left Heart Syndrome at One and Four Years of Age Comparing Hybrid and Norwood Procedure,” Annals of Thoracic and Cardiovascular Surgery 22(6): 375-377; Nagaraj. U. D., Evangelou, I. E., Donofrio, M. T., Vezina, L. G., McCarter, R., du Plessis, A. J., and Limperopoulos, C. 2015. “Impaired Global and Regional Cerebral Perfusion in Newborns with Complex Congenital Heart Disease,” Journal of Pediatrics 167(5): 1018-1024; Sun, L., Macgowan, C. K., Sled, J. G., Yoo, S., Manlhiot, C., and 8 others 2015. “Reduced Fetal Cerebral Oxygen Consumption is Associated with Smaller Brain Size in Fetuses with Congenital Heart Disease,” Circulation 131(15): 1313-1323; Panigrahy, A., Schmithorst, V. J., Wisnowski, J, L., Watson, C. G., Bellinger, D. C., Newburger, J. W., and Rivkin, M. J. 2015. “Relationship of White Matter Network Topology and Cognitive Outcome in Adolescents with d-transposition of the Great Arteries,” Neuroimage. Clinical 7:438-448; Bellinger, D. C., Watson, C. G., Rivkin, M. J., Robertson, R, L., Roberts, A. E., and 6 others 2015. “Neuropsychological Status and Structural Brain Imaging in Adolescents with Single Ventricle who Underwent the Fontan Procedure,” Journal of the American Heart Association (Oxford, England) 4(12). pii: e002302; Gaynor, J. W., Ittenbach, R. F., Gerdes, M., Bernbaum, J., Clancy, R. R., and 5 others 2014. “Neurodevelopmental Outcomes in Preschool Survivors of the Fontan Procedure,” Journal of Thoracic and Cardiovascular Surgery 147(4): 1276-1283; Li, X., Robertson, C. M., Yu, X., Cheypesh. A., Dinu, I. A., and Li, J. 2014. “Early Postoperative Systemic Inflammatory Response is an Important Determinant for Adverse 2-year Neurodevelopment-associated Outcomes after the Norwood Procedure,” Journal of Thoracic and Cardiovascular Surgery (St. Louis, Missouri) 148(1): 202-206; Sugimoto, A., Ota, N., Ibuki, K., Miyakoshi, C., Murata, M., Tosaka, Y., Yamazaki, T., and Sakamoto, K. 2013. “Risk Factors for Adverse Neurocognitive Outcomes in School-aged Patients after the Fontan Operation,” European Journal of Cardiothoracic Surgery 44(3): 454-461; Yamamoto, Y., Khoo, N. S., Brooks, P. A., Savard, W., Hirose, A., and Homberger, L. K. 2013. “Severe Left Heart Obstruction with Retrograde Arch Flow Influences Fetal Cerebral and Placental Blood Flow,” Ultrasound in Obstetrics and Gynecology 42(3): 294-299; Herberg, U. and Hovels-Gurich, H. 2012. “Neurological and Psychomotor Development of Foetuses and Children with Congenital Heart Disease-Causes and Prevalence of Disorders and Long-term Prognosis,” (in German with English abstract at Pubmed) Zeitschrift für Geburtshilfe und Neonatologie [Journal of Obstetrics and Neonatology] 216(3): 132-140; Sherlock, R, L., McQuillen, P. S., and Miller, S. P., with 18 collaborators 2009. “Preventing Brain Injury in Newborns with Congenital Heart Disease: Brain Imaging and Innovative Trial Designs,” Stroke 40(1): 327-332).

Because it eliminates the need for cardiopulmonary bypass surgery, sudden and metered compound vascular bypass, or sudden switched-organ transplantation may eliminate this other suspected culprit responsible for later cognitive deficits (see, for example, Knirsch, W., Liamlahi, R., Dave, H., Kretschmar, O., and Latal, B. 2016. “Neurodevelopmental Outcome of Children with Hypoplastic Left Heart Syndrome at One and Four Years of Age Comparing Hybrid and Norwood Procedure,” Annals of Thoracic and Cardiovascular Surgery (Tokyo, Japan) 22(6): 375-377). A preparatory hybrid procedure is followed by surgery that uses cardiopulmonary support.

Seen in this light, the avoidance of the Fontan procedure through heart transplantation that initiates normal circulation can result in critically improved cognitive ability to refine the later humanity of a baby born with a univentricular heart, and while transplantation has often been described as exchanging one disease for another due to the risk of rejection and the lifelong need to remain on immunosuppressants, these problems are being overcome with the development of relatively complication-free immunosuppressants which an implant prosthetic control system can release automatically, eliminating the problem of noncompliance (Morrissey, P. E., Flynn, M, L., and Lin, S. 2007. “Medication Noncompliance and Its Implications in Transplant Recipients,” Drugs 67(10): 1463-1481).

However, in other cases, cognitive and other organ deficits following surgical repair of the heart may be unavoidable or, as cited in the following paragraph, unavoidable from a practical standpoint, so that avoiding the complications which follow a Fontan procedure, adding postnatal maldevelopment to the intrinsic defect, is all the more important: for as suggested by the trisomy syndromes, where defects of the brain and heart, as well as other tissues and organs, often coexist, congenital heart and brain defects may be genetically and/or developmentally pleiotropic.

Pleiotropically based developmental defects typically affect different organ systems (see, for example, Izzo, A., Mollo, N., Nitti, M., Paladino, S., Cali, G., and 7 others 2018. “Mitochondrial Dysfunction in Down Syndrome: Molecular Mechanisms and Therapeutic Targets,” Molecular Medicine (Cambridge, Massachusetts) 24(1): 2; Singh, S., Kumar, R., Roy, B., Woo, M. A., Lewis, A., Halnon, N. J., and Pike, N. A 2018. “Regional Brain Gray Matter Changes in Adolescents with Single Ventricle Heart Disease,” Neuroscience Letters 665:156-162; Pike, N. A., Roy, B., Gupta, R., Singh, S., Woo, M. A., Halnon, N. J., Lewis, A. B., and Kumar, R. 2018. “Brain Abnormalities in Cognition, Anxiety, and Depression Regulatory Regions in Adolescents with Single Ventricle Heart Disease,” Journal of Neuroscience Research 96(6): 1104-1118; Edie, S., Zaghloul, N. A., Leitch, C. C., Klinedinst, D. K., Lebron, J., and 4 others 2018. “Survey of Human Chromosome 21 Gene Expression Effects on Early Development in Danio rerio,” Genes Genomes Genetics (Bethesda, Maryland) 8(7): 2215-2223; Benhaourech, S., Drighil, A., and Hammiri, A, E. 2016. “Congenital Heart Disease and Down Syndrome: Various Aspects of a Confirmed Association,” Cardiovascular Journal of Africa 27(5): 287-290; Ding, B. 2015. “How does a 1.5-Fold Increase in Gene Dosage in Chromosome 21 Cause the Pleiotropic Phenotypes in Down Syndrome?,” available Online at https://pdfs.semanticscholar.org/4266/9c536246bfffb12c5d089fe4c657c0e2e219.pdf or https://www.omicsOnline.org/open-access/how-does-a-15fold-increase-in-gene-dosage-in-chromosome-21-causesthe-pleiotropic-phenotypes-in-down-syndrome-2472-1115-1000e101.pdf Journal of Down Syndrome and Chromosome Abnormalities 1:1; Paaby, A, B. and Rockman, M. V. 2013. “The Many Faces of Pleiotropy,” Trends in Genetics 29(2): 66-73; Letourneau, A. and Antonarakis, S, E. 2012. “Genomic Determinants in the Phenotypic Variability of Down Syndrome,” Progress in Brain Research 197:15-28; Goldberg, C. S., Mussatto, K., Licht, D., and Wemnovsky, G. 2011. “Neurodevelopment and Quality of Life for Children with Hypoplastic Left Heart Syndrome: Current Knowns and Unknowns,” Cardiology in the Young (Cambridge, England) 21 Supplement 1 2:88-92; Deitz, S. L. and Roper, R. J. 2011. “Trisomic and Allelic Differences Influence Phenotypic Variability during Development of Down Syndrome Mice,” Genetics 189(4): 1487-1495; Antonarakis, S. E., Lyle, R., Dermitzakis, E. T., Reymond, A., and Deutsch, S. 2004. “Chromosome 21 and Down Syndrome: From Genomics to Pathophysiology,” Nature Reviews. Genetics 5(10): 725-738).

Methods already exist to reduce the aneuploid representation in in vitro fertilized ova (Amano, T., Jeffries, E., Amano, M., Ko, A. C., Yu, H., and Ko, M. S. 2015. “Correction of Down syndrome and Edwards Syndrome Aneuploidies in Human Cell Cultures,” DNA Research 22(5): 331-342). However, that gametes would become available for examination and modification in the laboratory, then fertilized only if confirmed normal, especially where no cause for suspicion is present, is doubtful as well as impractical.

Other approaches to remediation have also been proposed which have therapeutic potential, and practical application of such remedial measures are cited here because the placement of a prosthetic disorder response system can play a key role in achieving strict compliance with prescriptive guidelines (Izzo, A., Mollo, N., Nitti, M., Paladino, S., Cali, G., and 7 others 2018. “Mitochondrial Dysfunction in Down Syndrome: Molecular Mechanisms and Therapeutic Targets,” Molecular Medicine (Cambridge, Massachussets) 24(1): 2; Izzo, A., Manco, R., de Cristofaro, T., Bonfiglio, F., Cicatiello, R., and 6 others 2017. “Overexpression of Chromosome 21 miRNAs May Affect Mitochondrial Function in the Hearts of Down Syndrome Fetuses,” International Journal of Genomics 2017:8737649; Izzo, A., Nitti, M., Mollo, N., Paladino, S., Procaccini, C., and 11 others 2017. “Metformin Restores the Mitochondrial Network and Reverses Mitochondrial Dysfunction in Down Syndrome Cells,” Human Molecular Genetics 26(6): 1056-1069; Izzo, A., Manco, R., Bonfiglio, F., Cali, G., De Cristofaro, T., and 7 others 2014. “NRIP1/RIP140 [nuclear receptor interacting protein 1] siRNA [small interfering ribonucleic acid]-mediated Attenuation Counteracts Mitochondrial Dysfunction in Down Syndrome,” Human Molecular Genetics 23(16): 4406-4419; Piccoli, C., Izzo, A., Scrima, R., Bonfiglio, F., Manco, R. and 11 others 2013. “Chronic Pro-oxidative State and Mitochondrial Dysfunctions are more Pronounced in Fibroblasts from Down Syndrome Foeti with Congenital Heart Defects,” Human Molecular Genetics 22(6): 1218-1132).

Whereas pleiotropic congenital defects which affect organs other than the brain in combination with the heart can be dealt with through surgical repair or transplantation, the brain is the one organ likely to remain irreparable and irreplaceable. Pleiotropy is capable of differentially affecting numerous tissues and organs (see, for example, Potter, H. 2016. “Beyond Trisomy 21: Phenotypic Variability in People with Down Syndrome Explained by Further Chromosome Mis-segregation and Mosaic Aneuploidy,” Journal of Down Syndrome and Chromosome Abnormalities 2(1). pii: 109; Papavassiliou, P., Charalsawad, C., Rafferty, K., and Jackson-Cook, C. 2015. “Mosaicism for Trisomy 21: A Review,” American Journal of Medical Genetics. Part A 167A (1): 26- 39). Avoiding the abnormal perfusion following surgical repair has the potential to eliminate the addition of postnatal maldevelopment to the maldevelopment resulting from a genetic defect, which can pertain to any number of bodily organs and tissues.

b. The lungs (see, for example). Saul, D., Degenhardt, K., Iyoob, S. D., Surrey, L. F., Johnson, A. M., Johnson, M. P., Rychik, J., and Victoria, T. 2016. “Hypoplastic Left Heart Syndrome and the Nutmeg Lung Pattern in Utero: A Cause and Effect Relationship or Prognostic Indicator?,” Pediatric Radiology 46(4): 483-489; Gobergs, R., Salputra, E., and Lubaua, I. 2016. “Hypoplastic Left Heart Syndrome: A Review,” Acta Medica Lituanica 23(2): 86-98; Davies, R. R., Radtke, W., Bhat, M. A., Baffa, J. M., Woodford, E., and Pizarro, C. 2015. “Hybrid Palliation for Critical Systemic Outflow Obstruction: Neither Rapid Stage 1 Norwood nor Comprehensive Stage 2 Mitigate Consequences of Early Risk Factors,” Journal of Thoracic and Cardiovascular Surgery 149(1): 182-191; Davies, R. R., Radtke, W. A., Klenk, D., and Pizarro, C. 2014. “Bilateral Pulmonary Arterial Banding Results in an Increased Need for Subsequent Pulmonary Artery Interventions,” Journal of Thoracic and Cardiovascular Surgery 147(2): 706-712; Victoria, T. and Andronikou, S. 2014. “The Fetal MR Appearance of ‘Nutmeg Lung’: Findings in 8 Cases Linked to Pulmonary Lymphangiectasia,” Pediatric Radiology 44(10): 1237-1242; Amold, R. R., Loukanov, T., and Gorenflo, M. 2014. “Hypoplastic Left Heart Syndrome-Unresolved Issues,” Online, Frontiers in Pediatrics 2:125). Recent research reveals a genetic basis for at least certain instances of hypoplastic left heart syndrome (Durbin, M. D., Cadar, A. G., Williams, C. H., Guo, Y., Bichell, D. P., Su, Y. R., and Hong, C, C. 2017. “Hypoplastic Left Heart Syndrome Sequencing Reveals a Novel NOTCH1 Mutation in a Family with Single Ventricle Defects,” Pediatric Cardiology 38(6): 1232-1240), and if pleiotropic, the same genetic basis may also induce other malformities directly.

Arguing for a fetal heart transplant, the effect on the lungs of a defective heart in a neonate over no more than days after delivery may be so severe that the neonate can no longer be saved, at least not without an immediate heart-lung transplant, although injury to the remaining organ, glands, and tissues may deny much quality of life unless these too are soon replaced (Weidenbach, M., Caffier, P., Harisch, T., and Daehnert, I. 2006. “Hypoplastic Left Heart Syndrome with Intact Atrial Septum-Attempt of an Interventional Palliation by Ductal and Interatrial Stent Implantation,” Clinical Research in Cardiology 95(2): 110-114; Vlahos, A. P., Lock, J. E., McElhinney, D. B., and van der Velde, M, E. 2004. “Hypoplastic Left Heart Syndrome with Intact or Highly Restrictive Atrial Septum: Outcome after Neonatal Transcatheter Atrial Septostomy,” Circulation 109(19): 2326-2330; Reichart, B., Gulbins, H., Meiser, B. M., Kur, F., Briegel, J., and Reichenspurner, H. 2003. “Improved Results after Heart-Lung Transplantation: A 17-year Experience,” Transplantation 75(1): 127-132; Rychik, J., Rome, J. J., Collins, M. H., DeCampli, W. M., and Spray, T. L. 1999. “The Hypoplastic Left Heart Syndrome with Intact Atrial Septum: Atrial Morphology, Pulmonary Vascular Histopathology and Outcome,” Journal of the American College of Cardiology 34(2): 554-560).

While potentially valuable as a bridge to a heart transplant, where severe heart defects are addressed using relatively conventional techniques prenatally or immediately postnatally, the life of the baby is usually saved; however, without a heart transplant, that life may be short and distressed (see, for example, Generali, T., Hermuzi, A., Sajnach-Menke, M., Johnson, A., Crossland, D. S., and 4 others 2022. “Evolving Approach in Hypoplastic Left Heart Syndrome with Restrictive and Intact Septum,” World Journal for Pediatric and Congenital Heart Surgery, 13(3): 311-320; Hermuzi, A., McBrien, A., De Rita, F., McCheyne, A., Griselli, M., O'Sullivan, J. J., Hasan, A., and Crossland, D. S. 2016. “Hybrid Transatrial Stent Insertion for Left Atrial Decompression in Hypoplastic Left Heart Syndrome with Intact Atrial Septum” Catheterization and Cardiovascular Interventions 87(1): 109-116; Arai. S., Fujii. Y., Kotani, Y., Kuroko, Y., Kasahara, S., and Sano, S. 2015. “Surgical Outcome of Hypoplastic Left Heart Syndrome with Intact Atrial Septum,” Asian Cardiovascular and Thoracic Annals 23(9): 1034-1038; Como, A, F. 2015. “Functionally” Univentricular Hearts: Impact of Pre-natal Diagnosis,” Online, Frontiers in Pediatrics 3:15; Como, A, F. 2015. “Editorial: Univentricular Heart,” Online, Frontiers in Pediatrics 3:75; Suzuki, S., Kise, H., Kaga, S., Hoshiai, M., Koizumi, K., Hasebe, Y., Motohashi, S., and Matsumoto, M. 2015. “Hybrid Procedures for an Infant with Hypoplastic Left Heart Syndrome with Intact Atrial Septum,” General Thoracic and Cardiovascular Surgery 63(8): 472-475; Dave, H., Rosser, B., Knirsch, W., Hubler, M., Pretre, R., and Kretschmar, O. 2014. “Hybrid Approach for Hypoplastic Left Heart Syndrome and Its Variants: The Fate of the Pulmonary Arteries,” European Journal of Cardiothoracic Surgery 46(1): 14-19. Op cit. above in this section).

Thus, although recent developments reduce mortality, severe illness and an early death remain problems (see, for example, Jantzen, D. W., Moon-Grady, A. J., Morris, S. A., Armstrong, A. K., Berg, C., and 17 others 2017. “Hypoplastic Left Heart Syndrome with Intact or Restrictive Atrial Septum: A Report from the International Fetal Cardiac Intervention Registry,” Circulation September 1. pii: CirculationAHA. 116.025873; Hermuzi, A., McBrien, A., De Rita, F., McCheyne, A., Griselli, M., O'Sullivan, J. J., Hasan, A., and Crossland, D. S. 2016. “Hybrid Transatrial Stent Insertion for Left Atrial Decompression in Hypoplastic Left Heart Syndrome with Intact Atrial Septum,” Catheterization and Cardiovascular Interventions 87(1): 109-116; Sathanandam, S. K., Philip. R., Gamboa, D., Van Bergen, A., Ilbawi, M. N., and 4 others 2016. “Management of Hypoplastic Left Heart Syndrome with Intact Atrial Septum: A Two-centre Experience,” Cardiology in the Young (Cambridge, England) 26(6): 1072-1081; Weidenbach, M., Caffier, P., Harnisch, T., and Daehnert, I. 2006, Op cit.; Vlahos, A. P., Lock, J. E., McElhinney, D. B., and van der Velde, M, E. 2004; Rychik. J., Rome, J. J., Collins, M. H., DeCampli, W. M., and Spray, T. L. 1999. Op cit.).

c. The kidneys (see, for example, Opotowsky, A. R., Baraona, F. R., Me Causland, F. R., Loukas, B., Landzberg, E., and 3 others 2017. “Estimated Glomerular Filtration Rate and Urine Biomarkers in Patients with Single-ventricle Fontan Circulation,” Heart 103(6): 434-442; Athwani, V., Bhargava, M., Chanchlani. R., and Mehta, A. J. 2017. “Incidence and Outcome of Acute Cardiorenal Syndrome in Hospitalized Children,” Indian Journal of Pediatrics 84(6): 420-424; Sharma, S., Ruebner, R, L., Furth, S, L., Dodds, K. M., Rychik, J., and Goldberg, D. J. 2016. “Assessment of Kidney Function in Survivors following Fontan Palliation,” Congenital Heart Disease (Henderson, Nevada) 11(6): 630-636; Wong, J. H., Selewski, D. T., Yu. S., Leopold, K. E., Roberts, K. H., and 5 others 2016. “Severe Acute Kidney Injury following Stage 1 Norwood Palliation: Effect on Outcomes and Risk of Severe Acute Kidney Injury at Subsequent Surgical Stages,” Pediatric Critical Care Medicine 17(7): 615-623; Cooper, D. S., Kwiatkowski, D. M., Goldstein, S, L., and Krawczeski, C, D. 2016. “Acute Kidney Injury and Cardiorenal Syndromes in Pediatric Cardiac Intensive Care,” Pediatric Critical Care Medicine 17(8 Suppl 1): S250-S256; Savla, J., Lin, K. Y., Pradhan, M., Ruebner, R, L., Rogers, R. S., and 6 others 2015, Op cit.; Morgan, C., Al- Aklabi, M., and Garcia Guerra, G. 2015. “Chronic Kidney Disease in Congenital Heart Disease Patients: A Narrative Review of Evidence,” Canadian Journal of Kidney Health and Disease 2:27; Tromp. T. R., de Jonge, N., and Joles, J, A. 2015. “Left Ventricular Assist Devices: A Kidney's Perspective,” Heart Failure Reviews 20(4): 519-532; Patel, A. M., Adeseun, G. A., Ahmed, I., Mitter, N., Rame, J. E., and Rudnick, M, R. 2013. “Renal Failure in Patients with Left Ventricular Assist Devices,” Clinical Journal of the American Society of Nephrology 8(3): 484-496; Merz, W. M., Kübler, K., Fimmers, R., Willruth, A., Stoffel-Wagner, B., and Gembruch, U. 2013. “Cardiorenal Syndrome is Present in Human Fetuses with Severe, Isolated Urinary Tract Malformations,” Online, PLoS [Public Library of Science] One 8(5): e63664; Rosner, M. H., Rastogi. A., and Ronco, C. 2011. “The Cardiorenal Syndrome,” International Journal of Nephrology 2011:982092; Ronco, C., Mccullough, P., Anker, S. D., Anand, I., Aspromonte, N., and 23 others 2010. “Cardio-renal Syndromes: Report from the Consensus Conference of the Acute Dialysis Quality Initiative,” European Heart Journal 31(6): 703-711: Cruz, D. N. and Bagshaw, S. M. 2010. “Heart-kidney Interaction: Epidemiology of Cardiorenal Syndromes,” International Journal of Nephrology 2011: 351291; Anne, P., Du. W., Mattoo, T. K., and Zilberman, M. V. 2009. “Nephropathy in Patients after Fontan Palliation,” International Journal of Cardiology 132(2): 244-247).

d. The liver (see, for example, Goldberg, D. J., Surrey, L. F., Glatz, A. C., Dodds, K., O'Byrne, M, L., and 6 others 2017. “Hepatic Fibrosis is Universal Following Fontan Operation, and Severity is Associated with Time from Surgery: A Liver Biopsy and Hemodynamic Study,” Journal of the American Heart Association (Oxford, England) 6(5). pii: e004809; Dijkstra, H., Wolff, D., van Melle, J. P., Bartelds. B., Willems. T. P., and 6 others 2017. “Diminished Liver Microperfusion in Fontan patients: A Biexponential DWI [diffusion-weighted imaging] Study,” Online, PLoS [Public Library of Science] One 12(3): e0173149; Hilscher, M. B., Johnson, J. N., Cetta, F., Driscoll, D. J., Poterucha, J. J., and 3 others 2017. “Surveillance for Liver Complications after the Fontan Procedure,” Congenital Heart Disease 12(2): 124-132; Kotb. M. A., Abd, E. Satar, I., Badr, A. M., Anis, N. H., Abd, El Rahman Ismail, H., Hamza, A. F., and Abdelkader, H. M. 2017. “Pulmonary Hypertension and Cardiac Hypertrophy in Children Recipients of Orthotopic Living Related Liver Transplantation,” Journal of Advanced Research 8(6): 663-668; Chen, B., Schreiber, R. A., Human, D. G., Potts, J. E., and Guttman, O, R. 2016. “Assessment of Liver Stiffness in Pediatric Fontan Patients Using Transient Elastography,” Canadian Journal of Gastroenterology and Hepatology 2016:7125193; Greenway, S. C., Crossland, D. S., Hudson, M., Martin, S. R., Myers, R. P., and 3 others 2016. “Fontan-associated Liver Disease: Implications for Heart Transplantation,” Journal of Heart and Lung Transplantation (New York, New York) 35(1): 26-33; Wolff. D., van Melle, J. P., Dijkstra, H., Bartelds, B., Willems, T. P., and 5 others 2016. “The Fontan Circulation and the Liver: A Magnetic Resonance Diffusion-weighted Imaging Study,” International Journal of Cardiology 202:595-600; Josephus Jitta, D., Wagenaar, L. J., Mulder, B. J., Guichelaar, M., Bouman, D., and van Melle, J. P. 2016. “Three Cases of Hepatocellular Carcinoma in Fontan Patients: Review of the Literature and Suggestions for Hepatic Screening,” International Journal of Cardiology 206:21-26: Ford, R. M., Book, W., and Spivey, J, R. 2015. “Liver Disease Related to the Heart,” Transplantation Reviews (Orlando, Florida) 29(1): 33-37; Fouad, Y. M. and Yehia, R. 2014. “Hepato-cardiac Disorders,” World Journal of Hepatology 6(1): 41-54; Assenza, G. E., Graham, D. A., Landzberg, M. J., Valente, A. M., Singh, M. N., and 6 others 2013. “MELD-XI [Model for End-stage Liver Disease excluding INR (International Normalized Ratio of Prothrombin Time) score (function of creatinine and total bilirubin] Score and Cardiac Mortality or Transplantation in Patients after Fontan Surgery,” Heart (London) 99(7): 491-496; Moller, S. and Bernardi, M. 2013. “Interactions of the Heart and the Liver,” European Heart Journal 34(36): 2804-2811; Kavoliuniene, A., Vaitiekiene, A., and Cesnaite, G. 2013. “Congestive Hepatopathy and Hypoxic Hepatitis in Heart Failure: A Cardiologist's Point of View,” International Journal of Cardiology 166(3): 554-558; Asrani, S. K., Asrani, N. S., Freese, D. K., Phillips, S. D., Warnes, C. A., Heimbach, J., and Kamath, P. S. 2012. “Congenital Heart Disease and the Liver,” Hepatology (Baltimore) 56(3): 1160-1169; Moller, S., Dumcke, C. W., and Krag, A. 2009. “The Heart and the Liver,” Expert Reviews of Gastroenterology and Hepatology 3(1): 51-64).

A list of affected organs glands, and tissues can only be abridged: when circulation is abnormal, every organ and tissue in the body as an integral unit is directly affected, and because every organ is dependent upon the function of every other organ, the insult is compounded in every direction, the entire body becoming sicker and sicker. In preparation for a heart transplant, mother-fetus incompatibility may require preliminary cross-chimerization between the two, the process extended to the donor once a heart is found.

For this reason, it is preferable to receive the heart under machine support in situ within the donor. Conventional procedures include solid organ transplants, endarterectomies, and, with the understanding that such malformities originate inside the developing heart, the extracardiac components of discordances of the great arteries, where, to the extent that the discordance persists once the outflow arteries rise up from within the heart, in some instances, extracardiac reverse cross shunting may ameliorate transposed, or discordant, flow.

### Developmental Problems in Children Following Heart Transplantation

Much literature attests to developmental and psychiatric problems of children who had undergone a conventional heart transplant. However, no clear demarcation is or could have been made to distinguish such consequences as attributable to the procedure or to the injury that had been done to the child by a defective native heart prior to transplantation. Neither is the inevitable apprehension and distraction due to the precariousness of the postprocedural condition, the effect of continued therapy on school attendance, or adherence to the prescription regimen considered. Another factor that goes unaddressed is the genetically dictated comorbidity often associated with a severely malformed heart at birth that replacing the heart would not reverse, and it is reasonable to suspect that having delayed the procedure would only have allowed that collateral injury to continue.

Because it eliminates surgical trauma to both native and donor hearts, ischemia, the need for cardiopulmonary bypass, general anesthesia, and cardioplegia, a change to compound vascular bypass, or switched-organ heart transplantation should materially reduce if not eliminate any postoperative deficits resulting from the operation as such. Furthermore, the implanted prosthetic disorder response system that administered the switched-organ transplant also administers the automatic execution of the prescription, eliminating noncompliance as a detraction from a favorable outcome as well as a cause for concern and stress.

Significantly, that immunosuppressants are directly pipe-targeted to the blood supply of the graft organ or organs should reduce concerns as to heightened susceptibility to postoperative infection and malignancy as addressed above in section 2a, entitled Background. General. Heart failure is not directly related to congenital heart disease; however, genetically related measures to ameliorate the condition may be telling as to factors involved in congenital heart disease. With some 400 to 600 genes involved, a genetically malformed heart is seldom an isolated defect but rather a major expression of one or more pleiotropic genes which affect the development of more than the one organ or the structure of the body.

Numerous conditions such as trisomy 21, or Down syndrome, addressed above in this section, include congenital malformity of the heart (see, for example, Weaver, K. N., Chen, J., Shikany, A., White, P. S., Prada, C. E., Gelb, B. D., and Cnota, J, F. 2022. “Prevalence of Genetic Diagnoses in a Cohort with Valvar Pulmonary Stenosis,” Circulation: Genomic and Precision Medicine 15(4): e003635; Ko, J. M. 2015. “Genetic Syndromes Associated with Congenital Heart Disease,” Online, Korean Circulation Journal 45(5): 357-361; Oliveira, P, H. A., Souza, B. S., Pacheco, E. N., Menegazzo, M. S., Correa, I. S. and 5 others 2018. “Genetic Syndromes Associated with Congenital Cardiac Defects and Ophthalmologic Changes-Systematization for Diagnosis in the Clinical Practice,” (in English and Portuguese) Online, Arquivos Brasileiros de Cardiologia 110(1): 84-90′ Formigari, R., Michielon, G., Digilio, M. C., Piacentini, G., Carotti, A. and 3 others 2009. “Genetic Syndromes and Congenital Heart Defects: How is Surgical Management Affected?,” Online, European Journal of Cardio-Thoracic Surgery 35(4): 606-614; Michielon, G., Marino, B., Oricchio. G., Digilio, M. C., Iorio, F., and 3 others 2009. “Impact of DEL22q11, Trisomy 21, and Other Genetic Syndromes on Surgical Outcome of Conotruncal Heart Defects,” Online, Journal of Thoracic and Cardiovascular Surgery. Congenital Heart Disease 138(3): 565-570). Whether primarily defective and/or secondarily defective due to dependency upon the heart during development, an unsurvivably malformed heart is frequently accompanied by one or more other major organs such as the liver, kidneys, or lungs which also require to be replaced.

Continued research should lead to prenatal gene therapeutic and cellular methods for amelioration if not the prevention of both the congenital syndromes and the heart malformities these cause (see, for example, Truong, M. A., Cane-Gasull, P., and Lens, S. M, A. 2023. “Modeling Specific Aneuploidies: From Karyotype Manipulations to Biological Insights,” Online, Chromosome Research (Dordrecht, South Holland, Netherlands) 31(3): 25; Akutsu, S. N., Miyamoto, T., Oba, D., Tomioka, K., Ochiai. H., Ohashi, H., and Matsuura, S. 2022. “iPSC [induced pluripotent stem cells] Reprogramming-mediated Aneuploidy Correction in Autosomal Trisomy Syndromes,” PLoS [Public Library of Science] One (San Francisco, California) 17(3): e0264965; Chunduri, N. K., Barthel, K., and Storchova, Z. 2022. “Consequences of Chromosome Loss: Why Do Cells Need Each Chromosome Twice?,” Online, MDPI [Multidisciplinary Digital Publishing Institute] Cells (Basel, Switzerland) 11(9): 1530; Gou, Z., Zhou, Y., Jia. H., Yang, Z., Zhang, Q., and Yan, X. 2022. “Prenatal Diagnosis and mRNA Profiles of Fetal Tetralogy of Fallot,” Online, BioMed Central Pregnancy and Childbirth 22(1): 853; Kan, Z. Yan, W., Wang, N., Fang. Y., Gao. H., and Song, Y. 2022. “Identification of circ[ular] RNA-mi[cro] RNA-m[essinger] RNA Regulatory Network and Crucial Signaling Pathway Axis Involved in Tetralogy of Fallot” Online, Frontiers in Genetics 13:917454; Liu. S., Wei, W., Wang, P., Liu, C., Jiang. X., and 6 others 2022. “LOF [loss-of-function] Variants Identifying Candidate Genes of Laterality Defects Patients with Congenital Heart Disease,” Online, PLoS [Public Library of Science] Genetics 18(12): e1010530; Luo, F., Liu, W., and Bu, H. 2022. “MicroRNAs in Hypertrophic Cardiomyopathy: Pathogenesis, Diagnosis, Treatment Potential, and Roles as Clinical Biomarkers,” Heart Failure Reviews 27(6): 2211-2221; Meerschaut, I., Steyaert, W., Bove. T., Francois, K., Martens. T., and 11 others 2022. “Exploring the Mutational Landscape of Isolated Congenital Heart Defects: An Exome Sequencing Study Using Cardiac DNA,” Online, MDPI [Multidisciplinary Digital Publishing Institute] Genes (Basel, Switzerland) 13(7): 1214; Morton, S. U., Quiat, D., Seidman, J. G., and Seidman, C, E. 2022. “Genomic Frontiers in Congenital Heart Disease,” Online, Nature Reviews. Cardiology 19(1): 26-42; Ramachandran, V., Bhagavatheeswaran, S., Shanmugam, S., Vasudevan, M., Ragunathan, M., and 4 others 2022. “Deep Sequencing Unveils Altered Cardiac miR Nome in Congenital Heart Disease,” Molecular Genetics and Genomics 297(4): 1123-1139; Tabib, A., Talebi. T., Ghasemi, S., Pourirahim M., Naderi, N., Makeki, M., and Kalayinia. S. 2022. “A Novel Stop-gain Pathogenic Variant in FLT4 [Fms-related tyrosine kinase 4 protein encoded by the FLT4 gene] and a Nonsynonymous Pathogenic Variant in PTPN11 [Tyrosine-protein phosphatase non-receptor type 11 encoded in man by the PTPN11 gene] Associated with Congenital Heart Defects,” Online, BioMed Central European Journal of Medical Research (London, England) 27(1): 286; Tan, M., Wang, X., Liu, H., Peng. X., Yang. Y., and 4 others 2022. “Genetic Diagnostic Yield and Novel Causal Genes of Congenital Heart Disease,” Online, Frontiers in Genetics 13:941364; Wong, P. K., Cheah, F. C., Syafruddin, S. E., Mohtar, M. A., Azmi, N., Ng. P. Y., and Chua, E. W. 2021. “CRISPR [clustered regularly interspaced short palindromic repeats] Gene-editing Models Geared Toward Therapy for Hereditary and Developmental Neurological Disorders,” Frontiers in Pediatrics (Lausanne, Switzerland) 9:592571; Zhang, X., Gao, Y., Zhang, X. Zhang, X, Xiang. Y., and 3 others 2021. “FGDS-ASI is a Hub Inc [long noncoding] RNA ce [competing endogenous] RNA in Hearts with Tetralogy of Fallot which Regulates Congenital Heart Disease Genes Transcriptionally and Epigenetically,” Online, Frontiers in Cell and Developmental Biology 9:630634; Zhou, H., Tang, W., Yang, J., Peng, J., Guo, J., and Fan, C. 2021. “MicroRNA-related Strategies to Improve Cardiac Function in Heart Failure,” Online, Frontiers in Cardiovascular Medicine 8:773083; Akutsu, S. N., Fujita, K., Tomioka, K., Miyamoto. T., and Matsuura, S. 2020. “Applications of Genome Editing Technology in Research on Chromosome Aneuploidy Disorders,” Online, MDPI [Multidisciplinary Digital Publishing Institute] Cells (Basel, Switzerland) 9(1): 239; Das. S., Shah, R., Dimmeler, S., Freedman, J. E., Holley, C. and 6 others 2020. “Noncoding RNAs in Cardiovascular Disease: Current Knowledge, Toos and Technologies for Investigation and Future Directions: A Scientific Statement from the American Heart Association,” Online, Circulation: Cardiovascular Genetics/Genomic and Precision Medicine 13(4): e000062; Hoelscher, S. C., Stich, T., Diehm, A. Lahm., H., Dresen, M., and 13 others 2020. “miR-128a Acts as a Regulator in Cardiac Development by Modulating Differentiation of Cardiac Progenitor Cell Populations,” Online, MDPI [Multidisciplinary Digital Publishing Institute] International Journal of Molecular Sciences (Basel, Switzerland) 21(3): 1158; Ma. J., Chen, S., Hao, L., Sheng. W., Chen, W., and 4 others 2020. “Hypermethylation-mediated Down-regulation of Inc [long noncoding] TBX5-[T-box transcription factor 5-] AS1:2 [antisense 1:2] in Tetralogy of Fallot Inhibits Cell Proliferation by Reducing TBX5 Expression,” Online, Journal of Cellular and Molecular Medicine 24(11): 6472-6484; Nees, S. N. and Chung. W. K., 2020. “Genetic Basis of Human Congenital Heart Disease,” Online, Cold Spring Harbor Perspectives in Biology 12(9): a036749; Wilhelm, T., Said, M., and Naim, V. 2020. “DNA Replication Stress and Chromosomal Instability: Dangerous Liaisons,” Online, MDPI [Multidisciplinary Digital Publishing Institute] Genes (Basel, Switzerland) 11(6): 642; Colpaert, R. M. W. and Calore, M. 2019. “MicroRNAs in Cardiac Diseases,” Online, MDPI [Multidisciplinary Digital Publishing Institute] Cells (Basel, Switzerland) 8(7): 737; Duenas. A., Exposito, A., Aranega, A, and Franco, D. 2019. “The Role of Non-coding RNA in Congenital Heart Diseases,” Online, Journal of Cardiovascular Development and Disease 6(2): 15: Grunert. M., Appelt. S., Dunkel, I., Berger, F., and Sperling, S, R. 2019. Altered microRNA and Target Gene Expression Related to Tetralogy of Fallot,” Online, Scientific Reports 9(1): 19063; Li, B., Meng. X. and Zhang, L. 2019. “microRNAs and Cardiac Stem Cells in Heart Development and Disease,” Online, Drug Discovery Today 24(1): 233-240; Nagy, O., Barath, S., and Ujfalusi, A. 2019. “The Role of microRNAs in Congenital Heart Disease,” Online, eJIFCC [(Online) Journal of the International Federation of Clinical Chemistry and Laboratory Medicine] 30(2): 165-178; Williams, K., Carson, J., and Lo, C. 2019. “Genetics of Congenital Heart Disease,” Online, MDPI [Multidisciplinary Digital Publishing Institute] Biomolecules (Basel, Switzerland) 9(12): 879; Wilson, R., Ross, O., and Griksaitis, M. J. 2019. “Tetralogy of Fallot,” Online, British Journal of Anaesthesia Education 19(11): 362-369; Dill. T. L. and Naya, F. J. 2018. “A Hearty Dose of Noncoding RNAs: The Imprinted DLK1-DIO3 Locus in Cardiac Development and Disease,” Online, Journal of Cardiovascular Development and Disease 5(3): 37; Pierpont. M. E., Brueckner, M., Chung. W. K., Garg. V., Lacro, R. V., and 8 others 2018. “Genetic Basis for Congenital Heart Disease: Revisited: A Scientific Statement from the American Heart Association,” Online, Circulation 138(21): e653-e711. erratum 138(21): e713; Sallam, T., Sandhu, J., and Tontonoz, P. 2018. “Long Noncoding RNA Discovery in Cardiovascular Disease: Decoding Form to Function,” Online, Circulation Research 122(1): 155-166; Santoro M., Coi, A., Spadoni, I., Bianchi, F. and Pierini, A 2018. “Sex Differences for Major Congenital Heart Defects in Down Syndrome: A Population Based Study,” European Journal of Medical Genetics 61(9): 546-550; Wang. B., Shi, G., Zhu, Z., Chen, H., and Fu, Q. 2018. “Sexual Difference in Small RNA Expression in Tetralogy of Fallot” Online, Scientific Reports 8(1): 12847; Zhang, Y., Sastre, D., and Wang, F. 2018. “CRISPR/Cas9 Genome Editing: A Promising Tool for Therapeutic Applications of Induced Pluripotent Stem Cells,” Current Stem Cell Research and Therapy 13(4): 243-251; Doppler, S. A., Lange, R., Laugwitz, K. L, and Krane, M. 2017. “Cardiac Development from Current Understanding to New Regenerative Concepts,” Online, Journal of Thoracic Disease 9 (Supplement 1): S1-S4; Hoelscher, S. C., Doppler, S. A., Dresen, M., Lahm, H., Lange, R., and Krane, M. 2017. “MicroRNAs: Pleiotropic Players in Congenital Heart Disease and Regeneration,” Online, Journal of Thoracic Disease 9 (Supplement 1): S64-S81; Tian, J., An, X., and Niu, L. 2017. “Role of microRNAs in Cardiac Development and Disease,” Online, Experimental and Therapeutic Medicine 13(1): 3-8; Xie. D., Wang, H., Liu, Z., Fang, J., Yang. T., and 4 others 2017. “Perinatal Outcomes and Congenital Heart Defect Prognosis in 53313 Non-selected Perinatal Infants,” Online, PLoS [Public Library of Science] One 12(6): e0177229; Edwards, J. J. and Gelb, B, D. 2016. “Genetics of Congenital Heart Disease,” Online, Current Opinion in Cardiology 31(3): 235-241; Gelb, B, D. 2016. “Genetic Discovery for Congenital Heart Defects,” Chapter 51 in Nakanishi, T., Markwald, R. R., Baldwin, H. S., Keller, B. B., Srivastava, D., and Yamagishi, H. (eds.), Etiology and Morphogenesis of Congenital Heart Disease: From Gene Function and Cellular Interaction to Morphology. Tokyo, Japan: Springer Verlag: Grunert. M., Dom, C., Cui. H., Dunkel, I., Schullz, K., and 5 others 2016. “Comparative DNA Methylation and Gene Expression Analysis Identifies Novel Genes for Structural Congenital Heart Diseases,” Online, Cardiovascular Research 112(1): 464-477; Cowan, J, R. and Ware, S. M. 2015. “Genetics and Genetic Testing in Congenital Heart Disease,” Online, Indiana University Clinics in Perinatology 42(2): 373-393; Smith, T., Rajakaruna, C., Caputo, M., and Emanueli, C. 2015. “MicroRNAs in Congenital Heart Disease,” Online, Annals of Translational Medicine 3(21): 333; Bittel, D. C., Kibiryeva, N., Marshall, J. A., and O'Brien, J, E. Jr., 2014. “MicroRNA-421 Dysregulation is Associated with Tetralogy of Fallot,” Online, MDPI [Multidisciplinary Digital Publishing Institute] Cells (Basel, Switzerland) 3(3): 713-723; Gelb, B, D. and Chung. W. K. 2014. “Complex Genetics and the Etiology of Human Congenital Heart Disease,” Online, Cold Spring Harbor Perspectives in Medicine 4(7): a013953; Zhou, J., Dong, X., Zhou, Q., Wang. H., Qian, Y., and 3 others 2014. “microRNA Expression Profiling of Heart Tissue during Fetal Development,” Online, International Journal of Molecular Medicine (Athens, Greece) 33(5): 1250-1260; Gelb, B, D. 2013. “Recent Advances in Understanding the Genetics of Congenital Heart Defects,” Online, Current Opinion in Pediatrics 25(5): 561-566; French, V. M., van de Laar, I. M., Wessels, M. W., Rohe, C., Roos-Hesselink, J. W., and 24 others 2012. “NPHP4 [nephrocystin-4 encoding gene] Variants are Associated with Pleiotropic Heart Malformations,” Online, Circulation Research 110(412): 1564-1574; O'Brien, J, E. Jr., Kibiryeva, N., Zhou, X. G., Marshall, J. A., Lofland, G. K., and 3 others 2012. “Noncoding RNA Expression in Myocardium from Infants with Tetralogy of Fallot,” Online, Circulation: Cardiovascular Genetics/Genomic and Precision Medicine 5(3): 279-286; Bittel, D. C., Butler, M. G., Kibiryeva, N., Marshall, J. A., Chen, J., Lofland, G. K., and O'Brien, J, E. Jr., 2011. “Gene Expression in Cardiac Tissues from Infants with Idiopathic Conotruncal Defects,” Online, BioMed Central Medical Genomics 4:1; Bruneau, B. G., 2008. “The Developmental Genetics of Congenital Heart Disease,” Nature 451(7181): 943-948).

The effect on the psychosocial functioning of children affected by a congenitally defective heart is similar when the malformity is surgically ameliorated using conventional methods (see, for example, Kordopati-Zilou, K., Sergentanis, T., Pervanidou, P., Sofianou-Petraki, D., Panoulis, K., Vlahos, N., and Eleftheriades, M. 2022. “Neurodevelopmental Outcome in Tetralogy of Fallot: A Systematic Review,” Online, Children (Basel, Switzerland) 9(2): 264; Miatton, M., De Wolf, D., Francois, K., Thiery, E., and Vingerhoets, G. 2007. “Neuropsychological Performance in School- aged Children with Surgically Corrected Congenital Heart Disease,” Journal of Pediatrics 151(1): 73-78; Miatton, M., De Wolf, D., Francois, K., Thiery, E., and Vingerhoets, G. 2007. “Intellectual. Neuropsychological, and Behavioral Functioning in Children with Tetralogy of Fallot,” Online, Journal of Thoracic and Cardiovascular Surgery 133(2): 449-455).

The younger the patient at the time of the transplant, the less severe should be such collateral injury and the less severe the problems; however, the literature does not elaborate upon a breakdown in later performance based upon age. For this reason, it is reasonable to recommend that a severely malformed heart should be replaced by means of switched-organ transplantation as soon as possible (see, for example, Nassetta, K., Hussain, T., Gambetta, K., Le, K., O'Dwyer, L. C., and Badawy, S. M. 2022. “A Systematic Review of Adherence to Immunosuppression among Pediatric Heart Transplant Patients,” Online, Journal of Cardiovascular Development and Disease 9(5): 165; Anton, C. M., Drake, M. B., Butts, R. J., Cao. T., Rezaeizadeh, A., Trived, M. H., and Triplett, K. N. 2021. “Electronic Mental Health Screening in a Pediatric Heart Failure and Transplant Clinic,” Journal of Clinical Psychology in Medical Setting 28(4): 815-825; Balakrishnan, K. R., Rao, K, G. S. Subramaniam, G. K., Tanguturu, M. K., Arvind, A., and 9 others 2021. “Clinical Profiles and Risk Factors for Early and Medium-term Mortality following Heart Transplantation in a Pediatric Population: A Single-center Experience,” Online, Annals of Pediatric Cardiology 14(1): 42-52; Cushman, G. K., Stolz, M. G., Blount, R, L., and Reed, B. 2020. “Executive Functioning in Pediatric Solid Organ Transplant Recipients: A Meta-analytic Review,” Online, Transplantation 104(2): 357-366; Dipchand, A. I. and Laks, J, A. 2020. Op cit., above in section 2a, entitled Background. General: Sepke, M., Ferentzi, H., Disselhoff, V. S. U., and Albert, W. 2018. “Exploring the Developmental Tasks of Emerging Adults after Paediatric Heart Transplantation: A Cross-sectional Case Control Study” Online, British Medical Journal Open 8(11): e022461; Krishnamurthy, V., Freier Randall. C., and Chinnock, R. 2011. “Psychosocial Implications during Adolescence for Infant Heart Transplant Recipients,” Online, Current Cardiology Reviews 7(2): 123-134; Uzark. K., Spicer. R., and Beebe, D. W. 2009. “Neurodevelopmental Outcomes in Pediatric Heart Transplant Recipients,” Journal of Heart and Lung Transplantation (New York, New York) 28(12): 1306-1311; Chinnock, R, E. Freier. M. C., Ashwal. S., Prvonka-Jones, J., Shankel. T., Cutler, D., and Bailey, L. 2008. “Developmental Outcomes after Pediatric Heart Transplantation,” Journal of Heart and Lung Transplantation. Pediatric Transplantation (New York, New York) 27(10): 1079-1084; Wray J. and Radley-Smith, R. 2006. “Longitudinal Assessment of Psychological Functioning in Children after Heart of Heart-Lung Transplantation,” Journal of Heart and Lung Transplantation. Quality of Life (New York, New York) 25(3): 345-352; Wray J. and Radley-Smith, R. 2005. “Beyond the First Year after Pediatric Heart of Heart-Lung Transplantation: Changes in Cognitive Function and Behaviour,” Pediatric Transplantation 9(2): 170-177; Wray J. Long. T., Radley-Smith, R., and Yacoub, M. 2001. “Returning to School after Heart of Heart-Lung Transplantation: How Well Do Children Adjust?,” Online, Transplantation. Clinical Transplantation 72(1): 100-106).

2a(2). Inventive Content-Pertinent Fetal Diagnostics

Most applications of prosthetic disorder response systems relate to the automatic ambulatory diagnosis and treatment of serious chronic disorders addressed by internal medicine such as inborn errors of metabolism in a targeted and silent manner so that the patient is unaware of it. While direct pipeline targeting of medication considerably affects dosing and/or the character of electrostimulatory neuromodulation at the sites of disease, the essential information implemented through these otherwise conventional means remains strictly in accordance with conventional evidence-based medicine.

Inventive content-pertinent fetal diagnostics primarily relates to the detection of conditions that warrant solid organ transplantation, transposition of the great vessels, the replacement of anomalous vasculature, and conditions that warrant the implantation of an ambulatory automatic disorder response system. The prosthesis described herein for replacement of the lower urinary tract is incapable of growing in step with a tiny patient so that urinary tract diagnostics in utero and early life is not considered content-pertinent. Fetal diagnostics for the development of the liver, spleen, and gastrointestinal tract are relevant to the extent that these may require to be replaced using compound vascular bypass transplantation.

Implemented primarily as end-effectors in an automatic disorder response system, vascular valves and servovalves have to do with the semiautomated replacement through allotransplantation of solid organs, glands, major vessels, and rarely, vascularized composite tissue structures so congenitally malformed, missing, or impaired due to disease or injury as to have been rendered irreparable or not reparable to approximate normal function. In this capacity, compound vascular bypass, or sudden switched-organ transplantation eliminates much trauma and ischemia-reperfusion injury, and metered compound vascular bypass, or sudden switched-organ transplantation additionally avoids the shock of an abrupt confrontation with allogeneic tissue through pre- and midprocedural immune tolerance induction. As shown in FIG. 28, vascular valves also have utility in prostheses to replace the lower urinary tract

Unless expert repair is able to institute normal function essential for normal development, the repair will grow with a young patient to eliminate the need for revisions, and will minimize if not eliminate disfigurement, the replacement of native structures or tissues by these means will remain less likely to be rejected, and the implanted system will target immunosuppressives according to the schedule set forth in the prescription-program.

Unlike the substantial adherence to the principles of internal medicine involved in the automatic targeted release of medication, the automatic or manually supplemented administration of solid organ transplantation and the replacement of materially defective ductus such as major vessels are unconventional. Even less conventional is the application of such procedures prior to delivery. For this reason, some aspects of fetal diagnostics warrant mention.

Preferred methods for imaging different conditions appear among the references cited in the sections respective of each condition. Fetal diagnostics permits the detection of numerous metabolic and structural anomalies in utero, signaling the desirability, if at all possible, of temporarily removing, operating on, and returning a fetus with a serious congenital defect to the uterus thus interdicting the stillbirth or the serious secondary developmental consequences of having failed to intervene.

Critically for the life of the baby to be, when intervention is successful, this makes it possible to interdict a continued maldevelopment that left to continue even prior to birth would continue and could adversely affect every other organ and tissue in the body. However, using conventional methods, a fetus will often be too frail to withstand a traumatic procedure such as an organ transplant. Then the ability to stop further maldevelopment must be deferred until the patient attains the robustness necessary, which the defect itself might detain.

As will be described, compound vascular bypass, or switched-organ solid organ transplantation allows the replacement of a severely malformed heart, for example, substantially less traumatic than a conventional procedure making transplantation in so frail a patient not only feasible but timely in a way that will profoundly improve and extend the life of the patient. Furthermore, the main drawback to organ transplantation, that a failure to adhere to the prescribed regimen will result in rejection, is overcome with the aid of a fully implanted automatic disorder control system implanted in the infant once fully recovered.

At the same time, the vascular prostheses devised to expand with growth described herein allow the replacement of any segment along a defective major vessel which if not corrected to a flawless standard would result in global maldevelopment, and if left untreated, would result in death. Organ transplantation is fundamentally rendered more durable when the patient had been placed on life support before dying, so that the graft organ is spared the trauma associated with death; instead, it will continue to live in another individual. Also described are means for the extracardiac reversal of transposed great arteries and ischemia-free carotid endarterectomy.

Cardiac and pulmonary maldevelopment can be detected in the second trimester (see, for example, Heino, A., Morris, J. K., Garne, E., Baldacci, S., Barisic, I., and 13 others 2024. “The Association of Prenatal Diagnoses with Mortality and Long-term Morbidity in Children with Specific Isolated Congenital Anomalies: A European Register-based Cohort Study,” Online, Maternal and Child Health Journal (New York, New York) 28(6): 1020-1030; McEvoy, C., Leeds, H., Muralidaran, A., Johnson, A., Schilling, D., Parkhotyuk, K., and Shen, I. 2024. “Presurgical Pulmonary Function Tests in the First Few Days of Life in Neonates with Congenital Heart Disease, A Pilot Study,” [Preprint], Online, Research Square (Durham, North Carolina). February 13: rs. 3.rs-3938413; Agarwal, S., Mehollin-Ray, A., Sutton, V. R., and lacobas, I. 2023. “Prenatal Diagnosis of Vascular Anomalies” Prenatal Diagnosis(Chichester Sussex, England) 43(3): 318-327; Biko, D. M. and Fogel, M, A. 2023. “Fetal Cardiac MRI: Doppler US [ultrasound]-gated Cine Imaging in Complex Congenital Heart Disease,” Online, Radiology. Cardiothoracic Imaging (Oak Brook, Illinois) 5(1): e220314; Caro-Dominguez, P., Secinaro, A., Valverde, I., and Fouilloux, V. 2023. “Imaging and Surgical Management of Congenital Heart Diseases,” Pediatric Radiology (Berlin, Germany) 53(4): 677-694; Ju. J., Su. F., Chen, C., Sun, J., and Gao, Y. 2023. “Haplotype-assisted Noninvasive Prenatal Diagnosis of Genetic Diseases by Massively Parallel Sequencing of Maternal Plasma Cell-free DNA,” Methods in Molecular Biology (Clifton, New Jersey) 2590:287-294; Kuhle, H., Cho, S. K. S., Barber, N., Goolaub, D. S., Darby, J, R. T., and 4 others 2023. “Advanced Imaging of Fetal Cardiac Function,” Online, Frontiers in Cardiovascular Medicine (Lausanne, Switzerland) 10:1206138; Pederiva, F., Rothenberg, S. S., Hall, N., Ijsselstijn, H., Wong, K. K. Y. and 5 others 2023. “Congenital Lung Malformations,” Nature Reviews. Disease Primers (London, England) 9(1): 60; Sadlecki, P. and Walentowicz-Sadlecka, M. 2023. “Prenatal Diagnosis of Fetal Defects and Its Implications on the Delivery Mode,” Online, Open Medicine (Warsaw, Poland). 18(1): 20230704; Knapp. J., Tavares de Sousa, M., and Schonnagel, B. P. 2022. “Fetal Cardiovascular MRI—A Systemic Review of the Literature: Challenges, New Technical Developments, and Perspectives,” Rofo [Rontgenstrahlen Fortschritte] (Stuttgart, Germany) 194(8): 841-851; Kumar, P. and Bhatia, M. 2022. “Role of CT in the Pre- and Postoperative Assessment of Conotruncal Anomalies,” Online, Radiology. Cardiothoracic Imaging 4(3): e210089; Malho, A. S. Bravo-Valenzuela, N. J., Ximenes, R., Peixoto, A. B., and Araujo Junior, E. 2022. “Antenatal Diagnosis, of Congenital Heart Disease by 3D Ultrasonography Using Spatiotemporal Image Correlation with HDlive [high definition live rendering technique available in General Electric Voluson Model E10 ultrasound system] Flow an HDlive Flow Silhouette Rendering Modes,” Online, Ultrasonography 41(3): 578-596; Adair, J. D., Kelly, B., Schilling. D., Parkhotyuk, K., Gievers, L., and 3 others 2021. “Pulmonary Function Tests in Very Low Birth Weight Infants Screened for Pulmonary Hypertension: A Pilot Study,” Online, Journal of Pediatrics (St. Louis, Missouri) 237:221-226.e1; Sun, H. Y. 2021. “Prenatal Diagnosis of Congenital Heart Defects: Echocardiography,” Online, Translational Pediatrics (Hong Kong, China) 10(8): 2210-2224; Sun, L., van Amerom, J, F. P., Marini. D., Portnoy, S., Lee, F. T., and 9 others 2021. “MRI Characterization of Hemodynamic Patterns of Human Fetuses with Cyanotic Congenital Heart Disease,” Online, Ultrasound in Obstetrics and Gynecology 58(6): 824-836; Varunashree, N. D., Shankar, R., Navaneethan, P., Kumar, M., Benjamin S., and 3 others 2021. “Efficacy of Antenatal Ultrasound Examination in Diagnosis of Congenital Cardiac Anomalies in an Unselected Population: Retrospective Study from a Tertiary Centre,” Online, Journal of Obstetrics and Gynaecology of India 71(3): 277-284; Wang, F., Wang, C., Shi, J., Shan, Y., Miao. H., and 3 others 2021. “Lung Ultrasound Score Assessing the Pulmonary Edema in Pediatric Acute Respiratory Distress Syndrome Received Continuous Hemofiltration Therapy: A Prospective Observational Study,” Online, BioMed Central Pulmonary Medicine (London, England) 21(1): 40; Zhou, Y., Song, Z., Sun, L., Wang, Y., Lin, X., and Zhang, D. 2021. “Prenatal Diagnosis Nomograms: A Novel Tool to Predict Fetal Chromosomal Abnormalities in High-risk Patients,” Online, Risk Management and Healthcare Policy 14:4523-4535; Aguet, J., Seed. M., and Marini, D. 2020. “Fetal Cardiovascular Magnetic Resonance Imaging, Pediatric Radiology (Berlin, Germany) 50(13): 1881-1894; Bravo-Valenzuela, N. J., Peixoto, A. B., Junior, E, A. 2020. “Prenatal Diagnosis of Transposition of the Great Arteries: An Updated Review,” Online, Ultrasonography (Seoul, South Korea) 39(4): 331-339; Lin, T. Y., Sung, C. A., and Shaw, S. W. 2020. “The Application of Clinical Ultrasound in Fetal Therapy,” Online, Journal of Medical Ultrasound (Mumbai [Bombay], India) December 15:29(1): 1-2; Scott, H. D., Buchan, M., Chadwick, C., Field, C. J., Letourneau, N., and 3 others 2020. “Metabolic Dysfunction in Pregnancy: Fingerprinting the Maternal Metabolome Using Proton Nuclear Magnetic Resonance Spectroscopy,” Online, Endocrinology, Diabetes and Metabolism (Oxford, England) 4(1): e00201; van Nisselrooij, A, E, L., Teunissen, A. K. K., Clur, S. A., Rozendaal, L, Pajkrt. E., and 5 others 2020. “Why are Congenital Heart Defects being Missed?,” Online, Ultrasound in Obstetrics and Gynecology 55(6): 747-757; Patkee. P. A., Baburamani, A. A., Kyriakopoulou, V., Davidson, A., Avini, E., and 6 others 2019, Op cit., section 2a(1); Schrauben, E. M., Saini, B. S., Darby, J, R. T., Soo. J. Y., Lock, M. C., and 6 others 2019. “Fetal Hemodynamics and Cardiac Streaming Assessed by 4D Flow Cardiovascular Magnetic Resonance in Fetal Sheep,” Journal of Cardiovascular Magnetic Resonance 21(1): 8; Schlatterer, S. D., Mumick, J., Jacobs, M., White, L., Donofrio, M. T., and Limperopoulos, C. 2019. “Placental Pathology and Neuroimaging Correlates in Neonates with Congenital Heart Disease,” Science Reports 9(1): 4137; Alsaied. T., Tseng. S., King, E., Hahn, E., Divanovic, A., Habli, M., and Cnota, J. 2018. “Effect of Fetal Hemodynamics on Growth in Fetuses with Single Ventricle or Transposition of the Great Arteries,” Ultrasound in Obstetrics and Gynecology 52(4): 479-487; Bollini, S., Silini, A. R., Banerjee, A., Wolbank, S., Balbi, C., and Parolini, O. 2018. “Cardiac Restoration Stemming from the Placenta Tree: Insights from Fetal and Perinatal Cell Biology,” Online, Frontiers in Physiology 9:385; Bravo-Valenzuela, N. J., Peixoto, A. B., and Araujo Junior, E. 2018. “Prenatal Diagnosis of Congenital Heart Disease: A Review of Current Knowledge,” Online, Indian Heart Journal 70(1): 150-164; Courtney, J. A., Cnota, J. F., and Jones, H. N. 2018. “The Role of Abnormal Placentation in Congenital Heart Disease; Cause, Correlate, or Consequence?,” Online, Frontiers in Physiology 9:1045; Hunter, L, E. and Seale, A. N. 2018. “Prenatal Diagnosis of Congenital Heart Disease,” Echo Research and Practice 5(3): R81-R100; Karmacharya, S., Gagoski, B., Ning, L., Vyas. R., Cheng, H. H., and 5 others 2018, Op cit. above under section 2a(1), entitled Congenital Defects of the Heart and Other Organs: the Importance of Prompt Intervention; Levy, B. and Wapner, R. 2018. “Prenatal Diagnosis by Chromosomal Microarray Analysis,” Online, Fertility and Sterility (New York, New York) 109(2): 201-212; Rychik, J., Goff, D., Mckay, E., Mott. A., Tian, Z., Licht, D. J., and Gaynor, J. W. 2018. “Characterization of the Placenta in the Newborn with Congenital Heart Disease: Distinctions Based on Type of Cardiac Malformation,” Pediatric Cardiology 39(6): 1165-1171; Slodki, M., Axt- Fliedner. R., Zych-Krekora. K., Wolter, A., Kawecki, A., and 3 others 2018. “New Method to Predict Need for Rashkind Procedure in Fetuses with Dextro-transposition of the Great Arteries,” Ultrasound in Obstetrics and Gynecology 51(4): 531-536; Chou, C. Y. 2017. “Visualization of Ventricular Septal Defects on High-resolution Fetal Ultrasonography: Real or Artifact?,” Journal of Medical Ultrasound 25(1): 1-3; Godfrey, M. E., Friedman, K. G., Drogosz, M., Rudolph, A. M., and Tworetzky, W. 2017. “Cardiac Output and Blood Flow Redistribution in Fetuses with D-loop Transposition of the Great Arteries and Intact Ventricular Septum: Insights into Pathophysiology,” Ultrasound in Obstetrics and Gynecology 50(5): 612-617; Li, A., Peng, Z., and Zhang, C. 2017. “Comparison of Echocardiography and 64-multislice Spiral Computed Tomography for the Diagnosis of Pediatric Congenital Heart Disease,” Medical Science Monitor 23:2258-2266; Mackesy, M. M., Kalish, B. T., Tworetzky, W., Sanders, S., Al-Ibraheemi. A., and 4 others 2017. “Sonographic Pulmonary Abnormalities in Fetuses with Hypoplastic Left Heart Syndrome and Intact Atrial Septum Undergoing Attempted Atrial Septostomy in Utero,” Ultrasound Quarterly 33(1): 82-85; Tuo, G., Paladini, D., Montobbio, G., Volpe, P., Cheli. M., Calevo, M. G., and Marasini, M. 2017. “Prenatal Echocardiographic Assessment of Foramen Ovale Appearance in Fetuses with D-transposition of the Great Arteries and Impact on Neonatal Outcome,” Fetal Diagnosis and Therapy 42(1): 48-56; Parikh, L. I., Grantz, K, L., Iqbal, S. N., Huang, C. C., Landy, H. J., Fries, M. H., and Reddy, U. M. 2017. “Neonatal Outcomes in Fetuses with Cardiac Anomalies and the Impact of Delivery Route,” Online, American Journal of Obstetrics and Gynecology (New York, New York) 217(4): 469.e1-469.e12; Vigneswaran, T. V., Zidere. V., Miller, O. I., Simpson, J. M., and Sharland, G. K. 2017. “Usefulness of the Prenatal Echocardiogram in Fetuses with Isolated Transposition of the Great Arteries to Predict the Need for Balloon Atrial Septostomy,” American Journal of Cardiology 119(9): 1463-1467; Blanc, J., Fouron, J. C., Sonesson, S. E., Raboisson, M. J., Huggon, I., and 3 others 2016. “Ventricular Outputs, Central Blood Flow Distribution and Flow Pattern through the Aortic Isthmus of Fetuses with Simple Transposition of the Great Arteries,” Acta Obstetrica et Gynecologica Scandinavica 95(6): 629-634; Lai, C. W., Chau, A. K., and Lee, C. P. 2016. “Comparing the Accuracy of Obstetric Sonography and Fetal Echocardiography during Pediatric Cardiology Consultation in the Prenatal Diagnosis of Congenital Heart Disease,” Journal of Obstetrics and Gynaecology Research 42(2): 166-171; Peterson-Carmichael. S., Seddon, P. C., Cheifetz, I. M., Frerichs, I., Hall, G, L., and 10 others 2016. “Evaluation of Respiratory Mechanics and Function in the Pediatric and Neonatal Intensive Care Units,” Online, Annals of the American Thoracic Society (New York, New York) 13(2): S1-11; Tercanli, S. and Prüfer, F. 2016. “Fetal Neurosonogaphy: Ultrasound and Magnetic Resonance Imaging in Competition,” [in English] Ultraschall in der Medizin (Stuttgart, Germany) 37(6): 555-557; Wohlmuth, C., Wertaschnigg. D., Wieser, I., Arzt, W., and Tulzer, G. 2016. “Tissue Doppler Imaging in Fetuses with Aortic Stenosis and Evolving Hypoplastic Left Heart Syndrome before and after Fetal Aortic Valvuloplasty,” Ultrasound in Obstetrics and Gynecology 47(5): 608-615 (also cited above under 1. General); Hunter, L, E. and Simpson, J. M. 2014. “Prenatal Screening for Structural Congenital Heart Disease,” Nature Reviews Cardiology 11(6): 323-334; Wieacker, P. and Steinhard, J. 2010. “The Prenatal Diagnosis of Genetic Diseases, Deutsches Ärzteblatt International (Cologne, Germany) 107(48): 857-62; Davenport, M., Warne, S. A., Cacciaguerra, S., Patel, S., Greenough, A., and Nicolaides, K. 2004. “Current Outcome of Antenatally Diagnosed Cystic Lung Disease,” Journal of Pediatric Surgery (Philadelphia, Pennsylvania) 39(4): 549-556; Yagel. S., Weissman, A., Rotstein, Z., Manor, M., Hegesh. J., and 3 others 1997. “Congenital Heart Defects: Natural Course and in Utero Development,” Circulation 96(2): 550-555; Kugelman, A., Keens, T. G., deLemos. R., and Durand, M. 1995. “Comparison of Dynamic and Passive Measurements of Respiratory Mechanics in Ventilated Newborn Infants,” Pediatric Pulmonology (Hoboken, New Jersey) 20(4): 258-164; King, S., Pilling, D. W., and Walkinshaw, S. 1995. “Fetal Echogenic Lung Lesions: Prenatal Ultrasound Diagnosis and Outcome,” Pediatric Radiology (Berlin, Germany) 25(3): 208-210; Diukman, R. and Goldberg, J, D. 1993. “Prenatal Diagnosis of Inherited Metabolic Diseases,” Online, Western Journal of Medicine 159(3): 374-381; additional references cited above in section 2a. Background of the Invention. 1. General).

Later emergent or acquired defects in metabolism or hormonal function, for example, can be remedied through the implantation of a fully implanted automatic disorder response system to monitor and directly pipe-target medication to the originating and affected site or sites and/or by replacement of critically anomalous organs by using the compound vascular bypass method of solid organ or gland transplantation. Coordinated gravida, placenta, and fetus-implanted automatic disorder response system control is briefly addressed below in section 2b(2)(c), entitled Vascular Segment Prostheses: Inline Coupling Jackets and Bridging, or Tie-lines.

Severe maldevelopment of solid organs that would best be replaced before or after delivery using the compound vascular bypass technique is inventive content pertinent, and the detection of genetic disorders metabolic and structural such as an anomalous liver, spleen, and/or one or both kidneys, for example, in utero makes it possible to arrange for the defective organ or organs to be replaced as soon as possible, thus averting the secondary deficits in development that the defect or defects would otherwise cause. This is a significant factor in establishing deceased donor sustainment centers as addressed above in section 2a, entitled Background of the Invention. General, in the subsection thereof entitled Deceased Donor Sustainment Centers.

In utero genetic testing is now advanced (see, for example, D'Gama, A. M., Hills, S., Douglas, J., Young. V., Genetti, C. A., and 5 others 2024. “Implementation of Rapid Genomic Sequencing in Safety-net Neonatal Intensive Care Units,” Online, British Medical Journal Open (London, England) 14(2): e080529; D'Gama, A. M. and Agrawal, P, B. 2023. “Genomic Medicine in Neonatal Care: Progress and Challenges,” European Journal of Human Genetics (London, England) 31(12): 1357-1363; Findley, T. O., Parchem, J. G., Ramdaney, A., and Morton, S. U. 2023. “Challenges in the Clinical Understanding of Genetic Testing in Birth Defects and Pediatric Diseases,” Online, Translational Pediatrics (Hong Kong, China) 12(5): 1028-1040; Gordon, S., Umandap. C., and Langaker, M, D. 2023. “Prenatal Genetic Screening,” Online, Treasure Island, Florida: StatPearls Publishing Co; Wojcik, M. H., D'Gama, A. M., and Agrawal, P, B. 2023. “A Model to Implement Genomic Medicine in the Neonatal Intensive Care Unit,” Online, Journal of Perinatology (New York, New York) 43(2): 248-252; Kiani A K, Paolacci S. Scanzano P. Michelini S. Capodicasa N. and 16 others 2020. “Prenatal Genetic Diagnosis: Fetal Therapy as a Possible Solution to a Positive Test,” Online, Acta Biomedica (Fidenza, Parma, Italy) 91(13-S): e2020021; Wojcik, M. H., Reimers. R., Poorvu, T., and Agrawal, P, B. 2020. “Genetic Diagnosis in the Fetus,” Online, Journal of Perinatology (New York, New York) 40(7): 997-1006; Phadke, S. R., Puri, R. D., and Ranganath, P. 2017. “Prenatal Screening for Genetic Disorders: Suggested Guidelines for the Indian Scenario,” Online, Indian Journal of Medical Research (Mumbai [Bombay], India) 146(6): 689-699; Wilson, R. D., Gagnon, A., Audibert, F., Campagnolo, C., and Carroll, J. 2015. “Prenatal Diagnosis Procedures and Techniques to Obtain a Diagnostic Fetal Specimen or Tissue: Maternal and Fetal Risks and Benefits,” (in English and French) Journal of Obstetrics and Gynaecology Canada (Amsterdam, North Holland, Netherlands) 37(7): 656-668; Wieacker, P. and Steinhard, J. 2010. “The Prenatal Diagnosis of Genetic Diseases,” (in English and German) Online, Deutsches Arzteblatt International (Cologne, Germany) 107(48): 857-862).

As to the diagnosis of fetal liver disorders in utero, as of 2005. “Almost nothing is [was] known about fetal onset liver failure, and there is no literature addressing the subject,” (Whitington, P. F., Kelly. S., and Ekong, U, D. 2005. “Neonatal Hemochromatosis: Fetal Liver Disease Leading to Liver Failure in the Fetus and Newborn,” Pediatric Transplantation (Copenhagen, Denmark) 9(5): 640-645).

Directed to liver failure, the statement does not encompass all prenatal diagnosis of other problems other than liver failure which can be diagnosed during development (see, for example, Zermano. S., Novak. A., Vogrig, E., Parisi, N., and Driul. L. 2023. “GALD [Gestational Alloimmune Liver Disease]: New Diagnostic Tip for Early Diagnosis-A Case Report and Literature Review,” Frontiers in Reproductive Health (Lausanne, Switzerland) 5:1077304; Recio Rodriguez, M., Andreu-Vazquez, C., Thuissard-Vasallo, I. J., Cano Alonso, R., Bermejo Lopez, C., Tamarit Degenhardt, I., and Martinez Ten, P. 2020. “Real-life Diagnostic Accuracy of MRI in Prenatal Diagnosis,” Online, Radiology Research abd Practice (Cairo, Egypt) 2020:4085349; Reid. C., Arthurs, O. J., Calder, A. D., Sebire, N. J., and Shelmerdine, S, C. 2020. “The Significance of Internal Calcifications on Perinatal Post-mortem Radiographs,” Online, Clinical Radiology (Oxford, England) 75(7): 561.e25-561.e34; Shaughnessy, M. P., Spencer-Manzon, M., and Cowles, R, A. 2020. “Antenatally Detected Liver and Biliary Pathology,” Seminars in Pediatric Surgery (Philadelphia, Pennsylvania) 29(4): 150939; Pathak, S. and Grosu, L. 2019. “Case Series of Prenatal Diagnosis of Fetal Intrahepatic Lesions and Postnatal Outcome,” Online, Ultrasound (London, England) 27(2): 127-130; Davenport, M. and Hadzic, N. 2003. “Prenatal Diagnosis of Liver and Biliary Tract Disease,” Seminars in Neonatology (Amsterdam, North Holland, Netherlands) 8(5): 347-355; Simchen, M. J., Toi. A., Bona, M., Alkazaleh, F., Ryan, G., and Chitayat, D. 2002. “Fetal Hepatic Calcifications: Prenatal Diagnosis and Outcome,” American Journal of Obstetrics and Gynecology (New York, New York) 187(6): 1617-1622).

Inseparable from maldevelopment of the fetal liver is liver disease in the gravida (see, for example, Mei, J. Y. and Afshar, Y. 2023. “Hypertensive Complications of Pregnancy: Hepatic Consequences of Preeclampsia through HELLP [hemolysis, elevated liver enzymes, and low platelets] Syndrome,” Online, Clinical Liver Disease (Philadelphia, Pennsylvania) 22(6): 195-199; Sharma, A. V. and John, S. 2023. “Liver Disease in Pregnancy,” Online, Treasure Island, Florida: StatPearls Publishing Co.; Garcia-Romero, C. S., Guzman, C., Cervantes, A., and Cerbon, M. 2019. “Liver Disease in Pregnancy: Medical Aspects and Their Implications for Mother and Child,” Online, Annals of Hepatology (Tlapan, Mexico City, Mexico) 18(4): 553-562; Mikolasevic I. Filipec-Kanizaj T. Jakopcic I. Majurec I. Brncic-Fischer A, and 5 others 2018. “Liver Disease during Pregnancy: A Challenging Clinical Issue,” Online, Medical Science Monitor (Smithtown, New York) 15:24:4080-4090; Guntupalli, S, R. and Steingrub, J. S. 2005. “Hepatic Disease and Pregnancy: An Overview of Diagnosis and Management,” Critical Care Medicine (Philadelphia, Pennsylvania) 33(10 Supplement): S332-S339; Steingrub, J. S. 2004. “Pregnancy-associated Severe Liver Dysfunction,” Critical Care Clinics ((Philadelphia, Pennsylvania)) 20(4): 763-76, xi).

A collateral development is the use of fetal liver cells to treat liver disease (see, for example, Giancotti, A., D'Ambrosio, V., Como, S., Paino. C., Carpino, G., and 10 others 2022. “Current Protocols and Clinical Efficacy of Human Fetal Liver Cell Therapy in Patients with Liver Disease: A Literature Review,” Cytotherapy (London, England) 24(4): 376-384; Giancotti, A., Monti, M., Nevi, L., Safarikia, S., D'Ambrosio, V., and 10 others 2019. “Functions and the Emerging Role of the Foetal Liver into Regenerative Medicine,” Online, MDPI [Multidisciplinary Digital Publishing Institute] Cells (Basel, Switzerland) 8(8): 914; Semeraro R. Cardinale V. Carpino G, Gentile R. Napoli C. and 5 others 2013. “The Fetal Liver as Cell Source for the Regenerative Medicine of Liver and Pancreas,” Online, Annals of Translational Medicine (Hong Kong, China) 1(2): 13); Schmelzer, E., Zhang, L., Bruce, A., Wauthier, E., Ludlow, J., and 11 others 2007. “Human Hepatic Stem Cells from Fetal and Postnatal Donors,” Online, Journal of Experimental Medicine (New York, New York) 204(8): 1973-1987).

Kidney maldevelopment in utero is routinely detected with the aid of ultrasound (see, for example, Buijtendijk, M. F., Bet, B. B., Leeflang, M. M., Shah, H., Reuvekamp. T., and 10 others 2024. “Diagnostic Accuracy of Ultrasound Screening for Fetal Structural Abnormalities during the First and Second Trimester of Pregnancy in Low-risk and Unselected Populations,” Cochrane Database of Systematic Reviews (Chichester, West Sussex, England) 5(5): CD014715; Andelija, S and Tafti, D. 2023. “Sonography Fetal Assessment, Protocols, and Interpretation,” Online, Treasure Island, Florida: StatPearls Publishing Co.; Walawender, L., Becknell, B., and Matsell, D, G. 2023. Congenital Anomalies of the Kidney and Urinary Tract: Defining Risk Factors of Disease Progression and Determinants of Outcomes,” Pediatric Nephrology (Berlin, Germany) 38(12): 3963-3973; Syngelaki, A., Hammami, A., Bower, S., Zidere. V., Akolekar, R., and Nicolaides, K, H. 2019. “Diagnosis of Fetal Non-chromosomal Abnormalities on Routine Ultrasound Examination at 11-13 Weeks' Gestation,” Online, Ultrasound in Obstetrics and Gynecology (Chichester, West Sussex, England) 54(4): 468-476; Karim, J. N., Roberts, N. W., Salomon, L. J., and Papageorghiou, A. T. 2017. “Systematic Review of First-trimester Ultrasound Screening for Detection of Fetal Structural Anomalies and Factors that Affect Screening Performance,” Online, Ultrasound in Obstetrics and Gynecology (Chichester, West Sussex, England) 50(4): 429-441; Rosenblum, S., Pal. A., and Reidy. K. 2017. “Renal Development in the Fetus and Premature Infant,” Online, Seminars in Fetal and Neonatal Medicine (Amsterdam, North Holland, Netherlands) 22(2): 58-66; Nef. S., Neuhaus. T. J., Sparta, G., Weitz, M., Buder, K., and 4 others 2016. “Outcome after Prenatal Diagnosis of Congenital Anomalies of the Kidney and Urinary Tract,” European Journal of Pediatrics (Berlin, Germany) 175(5): 667-676, Tudorache, S., Cara, M., Iliescu, D. G., Stoica, A., Simionescu, C., Novac, L. V., and Cernea, D. 2016. Fetal Kidneys Ultrasound Appearance in the First Trimester-Clinical Significance and Limits of Counseling,” Online, Current Health Sciences Journal (Craiova, Romania) 42(1): 19-30; Yagel. S., Cohen, S. M., Porat, S., Daum, H., Lipschuetz, M., and 3 others 2015. “Detailed Transabdominal Fetal Anatomic Scanning in the Late First Trimester versus the Early Second Trimester of Pregnancy,” Journal of Ultrasound in Medicine (Oxford, England) 34(1): 143-149).

In children under five years of age thru adults, the spleen is transplanted in the context of multiorgan transplantation where the native spleen had to be removed leaving the patient susceptible to infection as well as in order to gain the advantage of the immune tolerance induction associated with multiple organ and tissue transplantation (see, for example, Kato, T., Tzakis, A. G., Selvaggi. G. Gaynor, J. J., Takahashi. H., and 10 others 2007. “Transplantation of the Spleen: Effect of Splenic Allograft in Human Multivisceral Transplantation,” Online, Annals of Surgery (Philadelphia, Pennsylvania) 246(3): 436-446; Kato, T., Kleiner, G., David, A., Selvaggi, G., Nishida, S., and 4 others 2006. “Inclusion of Spleen in Pediatric Multivisceral Transplantation,” Transplantation Proceedings (New York, New York) 38(6): 1709-1710) 246(3): 436-446)(see also Ashorobi, D. and Fernandz, R. 2024. “Asplenia,” Online, Treasure Island, Florida: StatPearls Publishing Co; Elchaninov. A., Vishnyakova, P., Lokhonina A., Kiseleva. V., Menyailo, E., and 8 others 2023. “Spleen Regeneration after Subcutaneous Heterotopic Autotransplantation in a Mouse Mmodel,” Online, BioMed Central Biological Research (London, England) 56(1): 15).

Reciprocally, in certain circumstances, a simultaneous splenectomy may result in a better outcome in relation to the transplantation of other organs (see, for example, Toshima, T., Harada, N., Itoh. S., Tomiyama, T., Toshida, K., and 7 others 2024. “What are Risk Factors for Graft Loss in Patients who Underwent Simultaneous Splenectomy during Living-donor Liver Transplantation?,” Transplantation (Hagerstown, Maryland) February 27; Yoshizumi. T., Itoh, S., Shimokawa, M., Inokuchi, S., Harada, N., and 8 others 2021. “Simultaneous Splenectomy Improves Outcomes after Adult Living Donor Liver Transplantation,” Journal of Hepatology (Amsterdam, North Holland, Netherlands) 74(2): 372-379). The contrary opinion is also represented (Ito. K., Akamatsu, N., Ichida, A., Ito. D., Kaneko, J., and 4 others 2016. “Splenectomy is Not Indicated in Living Donor Liver Transplantation,” Online, Liver Transplantation (Philadelphia, Pennsylvania) 22(11): 1526-1535).

In the treatment of congenital or acquired asplenia, for example, where a solitary spleen as opposed to a multiple allograft transplant is inadvisable, the implanted automatic disorder response system can function to monitor and dispense medicinal, mostly antibiotic prophylaxis, and as they develop, cellular, and genetic agents to simulate normal spleen function (see, for example, Lan, Y., Zhou, J., Qiu, T., Gong, X., and Ji. Y. 2023. “Refractory Kaposiform Lymphangiomatosis Relieved by Splenectomy,” Online, Frontiers in Pediatrics (Lausanne, Switzerland) 11:1203336; Butel-Simoes, G. I., Jones, P., Wood, E. M., Spelman. D., Woolley, I. J., and Ojaimi, S. 2022. Congenital Asplenia Study: Clinical and Laboratory Characterisation of Adults with Congenital Asplenia,” Annals of Hematology (Berlin, Germany) 101(7): 1421-1434; Lenti, M. V., Luu, S., Carsetti, R., Osier, F., Ogwang, R., and 6 others 2022. “Asplenia and Spleen Hypofunction,” Online, Nature Reviews. Disease Primers (London, England) 8(1): 71; Oszer, A., Babol-Pokora, K. Koltan, S., Pastorczak, A., and Mlynarski, W. 2021. “Germline 3p22.1 Microdeletion Encompassing RPSA [40S ribosomal protein SA encoding gene] Gene is an Ultra-rare Cause of Isolated Asplenia,” Online, BioMed Central Molecular Cytogenetics (London, England) 15:14(1): 51: Dinis- Ferreira, S., Lemos, C., Caldeira, M., Homem-Costa, M., da Luz Brazão, M., and Aveiro, F. 2020. “Isolated Congenital Asplenia in an Asymptomatic Patient: A Very Rare Diagnosis,” Online, European Journal of Case Reports in Internal Medicine (Milan, Italy) 7(4): 001429; Lee, G. M. 2020. “Preventing Infections in Children and Adults with Asplenia,” Online, Hematology (Washington, D C) 2020(1): 328-335; Queffelec. C., Billet, L., Duffau, P., Lazaro, E., Machelart, I., and 4 others 2020. “Prevention of Infection in Asplenic Adult Patients by General Practitioners in France between 2013 and 2016: Care for the Asplenic Patient in General Practice,” Online, BioMed Central Family Practice (London, England) 21(1): 163; Albrecht. T., Poss. K., Issaranggoon, Na. Ayuthaya. S., Triden, L., Schleiss, K, L., and Schleiss, M, R. 2019. “Case Report of Congenital Asplenia Presenting with Haemophilus Influenzae Type a (Hia) Sepsis: An Emerging Pediatric Infection in Minnesota,” Online, BioMed Central Infectious Diseases (London, England) 19(1): 947; Bolze, A., Mahlaoui, N., Byun, M., Turner, B., Trede. N., and 27 others 2013. “Ribosomal Protein SA Haploinsufficiency in Humans with Isolated Congenital Asplenia,” Online, Science (Washington, DC) 340(6135): 976-978; Mahlaoui, N., Minard-Colin, V., Picard, C., Bolze, A., Ku C, L., and 15 others 2011. “Isolated Congenital Asplenia: A French Nationwide Retrospective Survey of 20 Cases,” Journal of Pediatrics (St. Louis, Missouri) 158(1): 142-8, 148.e1).

By the same token, where one or more organs in addition to a spleen are transplanted so that taken together these provide a significant measure of immune tolerance induction, the implanted disorder response system can function to not only administer the transplantations but to provide follow-up monitoring and treatment tailored to each of the graft organs.

Most causes for asplenia or splenic inadequacy, or hyposplenism, are acquired such as caused by accidental trauma or disease, not congenital. In utero diagnosis of either condition once past the eighteenth week of gestation is by means of ultrasonography. Related techniques are under study (see, for example, Hall, M., Hutter, J., Suff, N., Avena Zampieri, C., Tribe, R. M., and 3 others 2022. “Antenatal Diagnosis of Chorioamnionitis: A Review of the Potential Role of Fetal and Placental Imaging,” Online, Prenatal Diagnosis(Chichester, Sussex, England) 42(8): 1049-1058; Roberto, M., Pasqualone, G., Di Donato, N, G. Malik, Z., Bhaiyat. S., and 5 others 2021. “A Novel Ultrasound-based Approach to Investigate Extramedullary Haematopoiesis in foetal Spleen,” Online, American Journal of Blood Research (Madison, Wisconsin) 11(1): 84-92; Shelmerdine, S. C., Hutchinson, J. C., Ward, L., Sekar, T., Ashworth, M. T., and 3 others 2020. “Feasibility of INTACT (INcisionless TArgeted Core Tissue) Biopsy Procedure for Perinatal Autopsy,” Online, Ultrasound in Obstetrics and Gynecology (Chichester, West Sussex, England) 55(5): 667-675; Diemert, A. and Tutschek, B. 2018. “Fetal Spleen,” in Copel, J, A. D'Alton, M. E., Feltovich, H., Gratacos, E., Krakow, D., and 3 others, Obstetric Imaging: Fetal Diagnosis and Care, Amsterdam, North Holland, Netherland: Elsevier Science Direct; Moreira, M., Bras. R., Gonçalves, D., Alencoao, I., Inocencio, G., Rodrigues, M., and Braga, J. 2018. “Fetal Splenomegaly: A Review,” Ultrasound Quarterly (Hagerstown, Maryland) 34(1): 32-33; Sepulveda, W., Ochoa, J. H., Cafici, D., Wong, A. E., Badano, F., Andreeva, E., and Andreeva, E. Y. 2018. “Splenic Cyst as a Rare Cause of Fetal Abdominal Cystic Mass: A Multicenter Series of Nine Cases and Review of the Literature,” Online, Ultrasound (London, England) 26(1): 22-31; Dankovcik, R., Urdzik, P., Lazar, I., Gresova, A., Radonak, J., and 4 others 2009. “Conservative Management in Three Cases of Prenatally Recognized Splenic Cyst Using 2D, 3D, Multi-slice and Doppler Ultrasonography,” Online, Fetal Diagnosis and Therapy (Basel, Switzerland) 26(3): 177-180; Aoki, S., Hata. T., and Kitao. M. 1992. “Ultrasonographic Assessment of Fetal and Neonatal Spleen,” American Journal of Perinatology (New York, New York) 9(5-6): 361-367; Hata. T., Aoki, S., Takamori, H., Hata, K., Murao. F., and Kitao. M. 1987. “Ultrasonographic in Utero Identification and Measurement of the Normal Fetal Spleen,” Gynecologie and Obstetric Investigation (Basel, Switzerland) 23(2): 124-128).

The study of splenic maldevelopment with the aid of monoclonal antibodies and the incisionless targeted core tissue biopsy technique are not limited as to trimester; however, based upon autopsies, these are valuable in terms of elucidating the embryology but not for the detection and the need for intervention in a clinical setting (see, for example, Timens, W., Rozeboom, T., and Poppema S. 1987. “Fetal and Neonatal Development of Human Spleen: An Immunohistological Study,” Online, Immunology (Oxford, England) 60(4): 603-609).

Where maldevelopment is slight, advancements in gene and cell therapy may eventually supplant the need for a heart transplant in order to achieve circulatory sufficiency. While likely, it is too early to specify the relations among remedial gene, cell, and medicinal agents and the method, time, and timing of administration to know in specific terms whether and how an implanted prosthetic disorder response system might facilitate treatment. An offhand view is that gene therapy would make use of targeting hyponeedles and cell therapy would involve the pipe-targeted delivery of the cells in liquid dispersion stored in drug reservoirs implanted subcutaneously in the pectoral region (see, for example, Shi, H. Y., Xie, M. S., Yang, C. X., Huang, R. T., Xue, S., and 3 others 2022. “Identification of SOXIS as a New Gene Predisposing to Congenital Heart Disease,” Online, MDPI [Multidisciplinary Digital Publishing Institute] Diagnostics (Basel, Switzerland) 12(8): 1917; Sabour, D., Machado, R. S. R., Pinto, J. P., Rohani, S., Sahito, R, G. A., and 3 others 2018. “Parallel Genome-wide Profiling of Coding and Non-coding RNAs to Identify Novel Regulatory Elements in Embryonic and Maturated Heart,” Molecular Therapy. Nucleic Acids 12:158-173; Cantu, C., Felker, A., Zimmerli, D., Prummel, K. D., Cabello, E. M., and 13 others 2018. “Mutations in Bcl9 and Pygo Genes Cause Congenital Heart Defects by Tissue-specific Perturbation of Wnt ([Wingless and Int-1] transduction pathways that gain entry into the cell through its surface receptors)/β-catenin Signaling,” Genes and Development 32(21-22): 1443-1458; Govindsamy, A., Naidoo, S., and Cerf. M, E. 2018. “Cardiac Development and Transcription Factors: Insulin Signalling, Insulin Resistance, and Intrauterine Nutritional Programming of Cardiovascular Disease,” Journal of Nutrition and Metabolism 2018:8547976; Zhu, H. 2016. “Forkhead Box Transcription Factors in Embryonic Heart Development and Congenital Heart Disease,” Life Sciences 144:194-201; Brade, T., Pane, L. S., Moretti, A., Chien, K. R., and Laugwitz, K. L. 2013. “Embryonic Heart Progenitors and Cardiogenesis,” Cold Spring Harbor Perspectives in Medicine 3(10): a013847).

However, the reversal of congenital heart defects through gene therapy lies well into the future, and the need for intensive diagnostics before birth and follow-up will then remain no less essential to alert the need for intervention; once bom, dependency upon a defective heart or any other malformed organ initiates a process of global bodily decline.

For this reason, the heart, the first organ to emerge as distinct, now discernible ab initio with the aid of fetal cardiac four chamber view magnetic resonance imaging using external cardiac gating as well as with ultrasound, for example, should be tracked vigilantly throughout fetal development, with the findings used to devise a contingency plan for repair, or if not satisfactorily repairable, then a heart transplant (see, for example, Goolaub, D. S., Roy, C. W., Schrauben, E., Sussman, D., Marini. D., Seed, M., and Macgowan, C. K. 2018. “Multidimensional Fetal Flow Imaging with Cardiovascular Magnetic Resonance: A Feasibility Study,” Journal of Cardiovascular Magnetic Resonance 20(1): 77; Kording, F., Schoennagel, B. P., de Sousa, M. T., Fehrs. K., Adam, G., Yamamura, J., and Ruprecht, C. 2018. “Evaluation of a Portable Doppler Ultrasound Gating Device for Fetal Cardiac M R Imaging: Initial Results at 1.5 T and 3 T,” Magnetic Resonance in Medical Sciences 17(4): 308-317; Kording, F., Yamamura, J., de Sousa, M. T., Ruprecht, C., Hedstrom, E. and 7 others 2018. “Dynamic Fetal Cardiovascular Magnetic Resonance Imaging Using Doppler Ultrasound Gating,” Journal of Cardiovascular Magnetic Resonance 20(1): 17; Kording, F., Ruprecht, C., Schoennagel, B., Fehrs, K., Yamamura, J., and 6 others 2018. “Doppler Ultrasound Triggering for Cardiac MRI at T T,” Magnetic Resonance in Medicine 80(1): 239-247; Steinhard. J., Entenmann, A., van der Valk, E., Schmitz, R., Heinig, J., Laser, K. T., and Michel, M. 2018. “The Transverse Four-chamber View for the Assessment of Atrial Tissue Deformation in the Fetus,” Online, PLoS [Public Library of Science] One 13(7): e0199581; Tavares de Sousa, M., Hecher, K., Yamamura, J., Kording, F., Ruprecht, C., and 4 others 2018. “Dynamic Fetal Cardiac Magnetic Resonance Four Chamber View Imaging Using Doppler Ultrasound Gating in the Normal Fetal Heart and in Congenital Heart Disease: Comparison to Fetal Echocardiography,” Ultrasound in Obstetrics and Gynecology October 31: Chaptinel, J., Yerly, J., Mivelaz, Y., Prsa, M., Alamo, L., and 5 others 2017. “Fetal Cardiac Cine Magnetic Resonance Imaging in Utero,” (Nature Research) Scientific Reports 7(1): 15540; Hutchinson, D., McBrien. A., Howley, L., Yamamoto, Y., Sekar, P., and 4 others 2017. “First-trimester Fetal Echocardiography: Identification of Cardiac Structures for Screening from 6 to 13 Weeks' Gestational Age,” Journal of the American Society of Echocardiography 30(8): 763-772; Roy, C. W., Seed, M., and Macgowan, C. K. 2017. “Accelerated MRI of the Fetal Heart Using Compressed Sensing and Metric Optimized Gating,” Magnetic Resonance in Medicine 77(6): 2125-2135; Sinkovskaya, E. 2017. “Advances in Fetal Cardiac Imaging,” Clinical Obstetrics and Gynecology 60(3): 636-655; Haris, K., Hedstrom, E., Bidhult, S., Testud. F., Maglaveras, N., and 4 others 2017. “Self-gated Fetal Cardiac MRI with Tiny Golden Angle iGRASP [iterative Golden-angle RAdial Sparse Parallel]: A Feasibility Study,” Journal of Magnetic Resonance Imaging 46(1): 207-217; Kording, F., Yamamura, J., Lund, G., Ueberle, F., Jung. C., Adam, G., and Schoennagel, B. P. 2017. “Doppler Ultrasound Triggering for Cardiovascular MRI at 3 T in a Healthy Volunteer Study,” Magnetic Resonance in Medical Sciences 16(2): 98-108; Huhta, J, C. 2016. “First-trimester Screening for Congenital Heart Disease,” Current Opinion in Cardiology 31(1): 72-77; Olsen, F. J., Bertelsen, L., de Knegt. M. C., Christensen, T. E., Veilstrup, N., and 3 others 2016. “Multimodality Cardiac Imaging for the Assessment of Left Atrial Function and the Association with Atrial Arrhythmias,” Circulation. Cardiovascular Imaging 9(10). pii: e004947; Junqueira, F. P., Gasparetto, T. D., and Werner, H. 2016. “MRI of the Fetal Heart: Comparison of the Obstetric Sonography and Fetal Magnetic Resonance Imaging Findings,” Journal of Cardiovascular Magnetic Resonance 18 (Supplement 1): 0117; Dong, S. Z. and Zhu, M. 2016. “MR Imaging of Fetal Cardiac Malposition and Congenital Cardiovascular Anomalies on the Four-chamber View,” Online, SpringerPlus 5(1): 1214; additional references are provided above under 1. Background of the Invention, a. General. (1). Congenital Defects of the Heart and Other Organs: The Importance of Prompt Correction).

In addition to these improvements in imaging, the detection of aberrant hemodynamic function and heart structure at 11 weeks gestation is now well developed, key elements in this capability being the advent of the nuchal translucency (NT) scan and ductus venosus (DV) blood color Doppler flow measurement (see, for example, Alldred, S. K., Takwoingi, Y., Guo, B., Pennant, M., Deeks. J. J., Neilson, J. P., and Alfirevic, Z. 2017. “First Trimester Ultrasound Tests Alone or in Combination with First Trimester Serum Tests for Down's Syndrome Screening,” Cochrane Database of Systematic Reviews 3: CD012600; Huhta, J, C. 2016. First-trimester Screening for Congenital Heart Disease,” Current Opinion in Cardiology 31(1): 72-77; Dmitrovic, A., Jeremic, K., Babic, U. M., Perovic, M., Mihailovic, T., Opric, D., Zecevic, N., and Gojnic-Dugalic, M. 2016. “Early Fetal Heart Ultrasonography as Additional Indicator for Chromosomopathies [chromosomal anomalies],” Clinical and Experimental Obstetrics and Gynecology 43(2): 245-249; Burger, N. B., Matias, A., Kok, E., de Groot, C. J., Christoffels, V. M., Bekker, M. N., and Haak, M, C. 2016. “Absence of an Anatomical Origin for Altered Ductus Venosus Flow Velocity Waveforms in First-trimester Human Fetuses with Increased Nuchal Translucency,” Prenatal Diagnosis 36(6): 537-544; Daniilidis, A., Balaouras, D., Chitzios, D., Balaouras, G., Capilna, M., and Asimakopoulos, E. 2015. “Variation of Ultrasound Findings in the First Trimester Examination of Recurrent Cases with Trisomy 21,” Online, Journal of Clinical Medicine Research (Brossard, Quebec, Canada) 7(6): 495-498; Schenone, A, L., Giugni, G., Schenone, M. H., Diaz, L., Bermudez, A., Majdalany, D., and Sosa- Olavarria, A. 2015. “Case Series: Fetal Pulmonary Vein A—wave Reversal: An Early Marker of Left-sided Cardiac Anomalies?,” American Journal of Perinatology Reports 5(1): e60-e66; Turan, S., Turan, O. M., Desai, A., Harman, C. R., and Baschat, A, A. 2014. “First-trimester Fetal Cardiac Examination Using Spatiotemporal Image Correlation, Tomographic Ultrasound and Color Doppler Imaging for the Diagnosis of Complex Congenital Heart Disease in High-risk Patients,” Ultrasound in Obstetrics and Gynecology 44(5): 562-567; Saleem, S. N. 2014. “Fetal MRI: An Approach to Practice: A Review,” Journal of Advanced Research 5(5): 507-523; Lipa, M., Samaha, R. B., Borowski, D., Wielgos, M., and Wegrzyn, P. 2014. “Fetal Hepatic Artery Flow Assessment in Prenatal Diagnostics—A Review of the Literature,” [in Polish with English abstract at Pubmed]. Ginekologia Polska [Gynecology in Poland] (85(7): 532-535; Borrell. A., Grande, M., Bennasar, M., Borobio, V., Jimenez, J. M., Stergiotou, I., Martinez, J. M., and Cuckle, H. 2013. “First-trimester Detection of Major Cardiac Defects with the Use of Ductus Venosus Blood Flow,” Ultrasound in Obstetrics and Gynecology 42(1): 51-57; Florjanski, J., Fuchs, T., Zimmer, M., Homola, W., Pomorski, M., and Blok, D. 2013. “The Role of Ductus Venosus Doppler Flow in the Diagnosis of Chromosomal Abnormalities During the First Trimester of Pregnancy,” Advances in Clinical and Experimental Medicine 22(3): 395-401; Chen, X., Chang, Y., Cui, H. Y., Ren, C. C., and Yu, B. Y. 2013. “Study on Several Ultrasound Markers Combined Maternal Serum Biochemical Markers to Screen Fetal Chromosomal Aneuploidy at 11 to 13 (+) 6 Weeks of Gestation,” [in Chinese with English abstract at Pubmed], Zhonghua Fu Chan Ke Za Zhi [Chinese Journal of Obstetrics and Gynecology] 48(11): 815-818; Karadzov-Orlic, N., Egic, A., Milovanovic, Z., Marinkovic, M., Damnjanovic-Pazin, B., and 4 others 2012. “Improved Diagnostic Accuracy by Using Secondary Ultrasound Markers in the First-trimester Screening for Trisomies 21, 18 and 13 and Turner Syndrome,” Prenatal Diagnosis 32(7): 638-643; Ghaffari, S. R., Tahmasebpour, A. R., Jamal, A., Hantoushzadeh, S., Eslamian, L., and 18 others 2012. “First-trimester Screening for Chromosomal Abnormalities by Integrated Application of Nuchal Translucency, Nasal Bone, Tricuspid Regurgitation and Ductus Venosus Flow Combined with Maternal Serum Free β-hCG [β-human chorionic gonadotropin] and PAPP-A [pregnancy-associated plasma protein-A]: A 5-year Prospective Study,” Ultrasound in Obstetrics and Gynecology 39(5): 528-534; Valenti, O., Di Prima, F. A., Renda, E., Faraci, M., Hyseni. E., De Domenico, R., Monte, S., and Giorgio, E. 2011. “Fetal Cardiac Function During the First Trimester of Pregnancy,” Journal of Prenatal Medicine 5(3): 59-62; Papatheodorou. S. I., Evangelou. E., Makrydimas, G., and Ioannidis, J. P. 2011. “First-trimester Ductus Venosus Screening for Cardiac Defects: A Meta-analysis,” British Journal of Obstetrics and Gynecology 118(12): 1438-1145; Timmerman, E., Oude Rengerink, K., Paikrt. E., Opmeer, B. C., van der Post. J. A., and Bilardo, C. M. 2010. “Ductus Venosus Pulsatility Index Measurement Reduces the False-positive Rate in First-trimester Screening,” Ultrasound in Obstetrics and Gynecology 36(6): 661-667; Timmerman, E., Clur, S. A., Pajkrt. E., and Bilardo, C. M. 2010. “First-trimester Measurement of the Ductus Venosus Pulsatility Index and the Prediction of Congenital Heart Defects,” Ultrasound in Obstetrics and Gynecology 36(6): 668-675; Martinez, J. M., Comas, M., Borrell. A., Bennasar, M., Gomez, O., Puerto, B., and Gratacos, E. 2010. “Abnormal First-trimester Ductus Venosus Blood Flow: A Marker of Cardiac Defects in Fetuses with Normal Karyotype and Nuchal Translucency,” Ultrasound in Obstetrics and Gynecology 35(3): 267-272; Perrone. A., Savelli, S., Maggi, C., Di Pietro, L., Di Maurizio, M., and 6 others 2008. “Magnetic Resonance Imaging Versus Ultrasonography in Fetal Pathology,” Radiologia Medica 113(2): 225-241).

With access to the fetus by hysterotomy, the effect of fetal surgery on the gravida tends to be somewhat more severe (Zamora, I. J., Ethun, C. G., Evans, L. M., Olutoye, O. O., Ivey, R. T., and 4 others 2013. “Maternal Morbidity and Reproductive Outcomes Related to Fetal Surgery,” Journal of Pediatric Surgery 48(5): 951-955; Golombeck, K., Ball, R. H., Lee, H., Farrell, J. A., Farmer, D, L., and 4 others 2006. “Maternal Morbidity after Maternal-fetal Surgery,” American Journal of Obstetrics and Gynecology 194(3): 834-819; Longaker, M. T., Golbus, M. S., Filly, R. A., Rosen, M. A., Chang, S. W., and Harrison, M, R. 1991. “Maternal Outcome after Open Fetal Surgery. A Review of the First 17 Human Cases,” Journal of the American Medical Association 265(6): 737-741).

Ventricular function and systolic dyssynchrony following heart transplantation can be assessed with three-dimensional echocardiography or tissue Doppler echocardiography (see, for example, Xing. B., Cao. A., Sun, W., and Hou, X. 2018. “Relationship between Left Ventricular Diastolic Dyssynchrony and Systolic Dyssynchrony in Hypertrophic Cardiomyopathy by Single-cardiac Real-time Three-dimensional Ultrasonography,” Experimental and Therapeutic Medicine 15(4): 3769-3774; Cai, Q. and Ahmad, M. 2015. “Left Ventricular Dyssynchrony by Three-dimensional Echocardiography: Current Understanding and Potential Future Clinical Applications,” Echocardiography 32(8): 1299-1306; Tani, T., Sumida, T., Tanabe, K., Ehara, N., Yamaguchi, K., and 7 others 2012. “Left Ventricular Systolic Dyssynchrony Index by Three-dimensional Echocardiography in Patients with Decreased Left Ventricular Function: Comparison with Tissue Doppler Echocardiography,” Echocardiography 29(3): 346-352).

Color Doppler ultrasonography, computed tomography, magnetic resonance imaging, and arterial or direct portography (radiography of the hepatic portal system and circulation with the aid of radiopaque contrast material), make it possible to diagnose anomalous development in progress of the portal venous system (Gallego, C., Velasco, M., Marcuello, P., Tejedor, D., De Campo, L., and Friera, A. 2002. “Congenital and Acquired Anomalies of the Portal Venous System,” RadioGraphies 22(1): 141-159).

2a(3). Remotely Procured Graft Organ Preservation

While exceptions can be made when the graft organ is properly transported, whenever possible, compound vascular switch organ transplantation requires the compresence of both donor and recipient. To facilitate this proximity, the establishment by large medical centers of deceased donor sustainment centers is recommended above in section 2a, entitled Background of the Invention. General, in the subsection thereof entitled Deceased Donor Sustainment Centers. This calls for creating a special unit where all suitable donors in the greater area having consented to organ donation are collected together for life support begun prior to death along with other support such as endocrinal, medicinal, and normothermal continued until their usable organs have been harvested.

Using either the sudden or metered switch technique when the donor and recipient can be positioned side by side, the graft organ is excised, or harvested, only after the circulation, to include each blood supply and venous drainage vessel of the recipient, has been switched from his own diseased organ to the corresponding vessels of the graft organ, in the donor—a capability made possible by vascular valves or servovalves under the unified control of a microcontroller or master control microprocessor. In circulatory system transfer thus, the graft organ is continuously perfused and never undergoes ischemia. When the graft organ is switched into the circulatory system of the recipient before the diseased recipient and donor organs are harvested, zero ischemic time, reperfusion, or reperfusion injury can occur.

In metered compound vascular bypass, or sudden switched-organ transplantation, transfer of the graft organ from the donor to the recipient is made to progress gradually. Transfer of a gradually increasing proportion of blood through the graft organ and the donor from the recipient to the donor is used to achieve a measure of increasing reciprocal cross-chimerization between the donor and the recipient, which process progresses in step with the degree to which the graft organ has been transferred to the recipient.

The blood of the recipient circulating throughout his own body and to a lesser extent throughout the circulatory system of the donor, reciprocal cross-chimerization and immune tolerance induction are accomplished at the same rate as the graft organ is transferred from the donor to the recipient. This cross transfusion, optimized when somatic support uses blood of the recipient, can be supplemented with the addition of exogenous agents as the transfer in blood flow proceeds. When the donor expires in the center, machine somatic support allows the organs of a deceased donor to effectively remain physiologically active for up to three months without the direct stimulatory measures delineated above to preserve respiration and regular circulation, and perhaps body temperature and endocrine function past death.

The terms ‘perfusion machine’ and ‘machine perfusion’ denote an apparatus that maintains the flow of blood through a graft organ after it has been harvested. Unless valve-jackets are used, perfusion during excision from the donor or anastomosis is interrupted, with the degree of reperfusion injury proportional to the time elapsed from connection to reconnection. Cardiopulmonary, or heart and lung bypass machines, and extracorporeal membrane oxygenators are also perfusion machines, but in the fundamentally different sense of providing intact somatic support, or supporting the body as a whole.

With either, once the recipient is identified, the blood used to perfuse the graft organ or body of the donor should be partially if not entirely that of the recipient by means of sudden compound vascular bypass. In many cases, the donor is discovered having already died, making the optimization of the organs for transplantation of central importance. In this case, information on organ preservation through ex vivo perfusion and storage in a static or flowing chilled perfusate pending transplantation is essential, the loss of many graft organs the alternative.

Such notwithstanding, the main object in furnishing information on ex vivo preservation is to document the essential inferiority of allowing the donor to finally die prior to transfer of the graft organ or organs to the recipient. Compresence of the recipient with the donor to allow reciprocal cross-circulation is decidedly preferable to the use of an ex vivo perfusion machine, which in ordinary use cuts off the entire body of the donor and donor circulatory system from confluence with the blood of the recipient, likely reducing the degree of donor-recipient cross microchimerization in proportion to the duration of reciprocal cross-circulation.

Nevertheless, when time allows, rather than to allow the use of a static or flowing chilled perfusate other than the circulating blood of the donor on life support with ancillary such as endocrinal and temperature support by medical attendants (see, for example, Shetty, V, L., Mali, S. S., Shetty, S. V., and Shinde, P, D. 2017, Op cit., above in section 2a, entitled Background. General), the machine can be primed with a portion of the circulating blood of the donor on life support.

Such should allow a degree of cross-chimerization and acclimation intermediate between connection of the recipient to the donor as compresent over as long an interval judged optimal, as is preferred, and the removal of the graft organ from the donor for placement in the ex vivo perfusion machine with a nonmicrochimerized, or neutral perfusate. Ischemia-reperfusion injury ensues whether oxygen deprivation had arisen during organ transplantation or following the obstruction of blood flow to an organ due to a thromboembolus, for example, such instances including cerebral and myocardial infarctions, as well as an embolic strokes and peripheral vascular disease, for example.

Essentially, the key to eliminating ischemia-reperfusion injury as well as encouraging immune tolerance induction is the elimination of graft organ preservation through the application of a compound vascular bypass, or switched organ technique, in which the circulation of the recipient is switched from his own native organ to the graft in a life support sustained donor through the seamless transfer of the graft organ from the circulatory system of the donor directly into the circulatory system recipient, immune tolerance attained by extending the transfer process during metered reciprocal cross-circulation with the addition of immune tolerance-inducing drugs if necessary.

Even when the donor has been identified and is not remote, conventional preservation is markedly inferior to the donor preservation centers described above in section 2a, entitled Background of the Invention. General, in the subsection thereof entitled Deceased Donor Sustainment Centers, and metered compound vascular bypass method described below in section 2b(1)(b), entitled Metered Compound Vascular Bypass, or Metered Switched-organ, Transplantation, (see, for example, Casanova, D., Castillo, F., and Minambres, E. 2022. “Multiorgan Retrieval and Preservation of the Thoracic and Abdominal Organs in Maastricht III Donors,” Online, World Journal of Transplantation (Pleasanton, California) 12(5): 83-87; Lepoittevin, M., Giraud, S., Kerforne, T., Barrou, B., Badet, L., and 10 others 2022. “Preservation of Organs to be Transplanted: An Essential Step in the Transplant Process,” Online, MDPI [Multidisciplinary Digital Publishing Institute] International Journal of Molecular Sciences (Basel, Switzerland) 23(9): 4989; Mico-Camero, M., Zaouali, M. A., Rojani-Alfonso, C., Maroto-Serrat, C., Ben Abdennebi, H., and Peralta, C. 2022. Op cit. above in section 2a, entitled Background. General; Rosen, R. D., Singh, A., and Burns, B. 2022. “Trauma Organ Procurement,” Online, Treasure Island, Florida: StatPearls Publishing Co.; Schenk, A. D., Logan, A. J., Sneddon, J. M., Faulkner, D., Han, J, L., Brock, G. N., and Washburn, W. K. 2022. “Textbook Outcome as a Quality Metric in Living and Deceased Donor Kidney Transplantation,” Online, Journal of the American College of Surgeons 235(4): 624-642; Zhang, M. Y., Dugbartey, G. J., Juriasingani, S., Akbari, M., Liu, W. and 3 others 2022. “Sodium Thiosulfate-supplemented UW [University of Wisconsin] Solution Protects against Prolonged Cold Ischemia-Reperfusion Injury in a Murine Model of Syngeneic Kidney Transplantation,” Online, Biomedicine and Pharmacotherapy 145:112435; Howard, M. C., Nauser, C, L., Farrar, C. A., and Sacks, S, H. 2021. “Complement in Ischaemia-Reperfusion Injury and Transplantation,” Online, Seminars in Immunopathology 43(6): 789-797. erratum 2022 44(3) 391; Zheng. H., Su. Y., Zhu, C., Quan, D. Skaro, A. I., and 6 others 2021. “An Addition of U0126 [a mitogen- activated protein MEK1 and MEK2 kinase inhibitor] Protecting Heart Grafts from Prolonged Cold Ischemia-Reperfusion Injury in Heart Transplantation: A New Preservation Strategy,” Online, Transplantation 105(2): 308-317; Rijkse, E., IJzermans, J. N. M, and Minnee, R, C. 2020. “Machine Perfusion in Abdominal Organ Transplantation: Current Use in the Netherlands,” Online, World Journal of Transplantation (Pleasanton, California) 10(1): 15-28; Soares, R. O. S., Losada, D. M., Jordani, M. C., Evora, P., and Castro-E-Silva, O. 2019. “Ischemia/Reperfusion Injury Revisited: An Overview of the Latest Pharmacological Strategies,” Online, MDPI [Multidisciplinary Digital Publishing Institute] International Journal of Molecular Sciences (Basel, Switzerland) 20(20): 5034; Tingle, S., Figueiredo, R. S., Moir, J. A., Goodfellow, M., Talbot, D., and Wilson, C, H. 2019. “Machine Perfusion Preservation versus Static Cold Storage for Deceased Donor Kidney Transplantation,” Online, Cochrane Database of Systematic Reviews 3: CD011671; Hentia, C., Rizzato, A., Camporesi, E., Yang, Z., Muntean, D. M., Sandesc, D. and Bosco, G. 2018. “An Overview of Protective Strategies against Ischemia/Reperfusion Injury: The Role of Hyperbaric Oxygen Preconditioning,” Online, Brain and Behavior 8(5): e00959; Kalogeris. T., Baines, C. P., Krenz, M., and Korthuis, R. J. 2016. “Cell Biology of Ischemia/Reperfusion Injury,” Online, International Review of Cell and Molecular Biology 298:229-317; Kalogeris, T., Baines, C. P., Krenz, M., and Korthuis, R. J. 2016. “Ischemia/Reperfusion,” Online, Comprehensive Physiology (Wiley, Hoboken, New Jersey) 7(1): 113-170; Goto. R., Issa, F., Heidt, S., Taggart, D. and Wood, K. J. 2013. “Ischemia- Reperfusion Injury Accelerates Human Antibody-mediated Transplant Vasculopathy,” Online, Transplantation 96(2): 139-145; So. M., Lee. T. D., and Hancock Friesen, C. L. 2013. “Neutrophils are Responsible for Impaired Medial Smooth Muscle Cell Recovery and Exaggerated Allograft Vasculopathy in Aortic Allografts Exposed to Prolonged Cold Ischemia,” Journal of Heart and Lung Transplantation (New York, New York) 32(3): 360-367; Gareau, A. J., Nashan, B., Hirsch, G. M., and Lee, T, D. 2012. “Cyclosporine Immunosuppression Does Not Prevent the Production of Donor-specific Antibody Capable of Mediating Allograft Vasculopathy,” Journal of Heart and Lung Transplantation (New York, New York) 31(8): 874-880; Devitt, J. J., King, C, L., Lee. T. D., and Hancock Friesen, C. L. 2011. “Early Innate Immune Events Induced by Prolonged Cold Ischemia Exacerbate Allograft Vasculopathy,” Online, BioMed Central Journal of Cardiothoracic Surgery 6:2: Eltzschig, H. K. and Eckle, T. 2011. “Ischemia and Reperfusion—From Mechanism to Translation,” Online, Nature Medicine 17(11): 1391-1401; additional references cited below).

That more recent guidance on preservation of heart grafts recommends the use of a continuously chilled static or flowing perfusate such as a cardioplegia solution in an ex vivo sustainment transport container over the immersion technique gains nothing compared to a direct organ transfer technique as described herein carried out in a center prepared to perform such a procedure (see, for example, Das, I., Pham, S. M., Perry, D. K., and Croome, K. P. 2024. “The Use of Ex situ Normothermic Machine Perfusion in Combined Cardiac and Liver Transplantation Procedures,” Online, Transplantation Direct (Philadelphia, Pennsylvania) 10(2): e1574; Alomari, M., Garg. P., Yazji, J. H., Wadiwala, I. J., Alamouti-Fard, E., and 3 others 2022. “Is the Organ Care System (OCS) Sill the First Choice with Emerging New Strategies for Donation after Circulatory Death (DCD) in Heart Transplant?,” Online, Cureus (Palo Alto, California) 14(6): e26281; Joshi, Y. Villaneuva, J., Gao, L., Hwang. B., Zhao. C., and 4 others 2022. “Donation after Circulatory Death: A New Frontier,” Online, Current Cardiology Reports 24(12): 1973-1981; Kaliyev R., Lesbekov, T., Bekbossynova, M., Nurmykhametova, Z., Medressova, A., and 4 others 2022. “Successful Heart Transplantation after 7 Hours of Cold Storage and Paracorporeal Donor Heart Resuscitation,” Online, Transplant International 35:10740; Qin, G., Jernryd. V., Sjoberg. T., Steen, S. and Nilsson, J. 2022. “Machine Perfusion for Human Heart Preservation: A Systematic Review,” Online, Transplant International 35:10258; Kaliyev R., Lesbekov, T., Bekbossynov, S., Bekbossynova, M., Nurmykhametova, Z., and 8 others 2019. “Comparison of Custodiol vs Warm Blood Cardioplegia and Conditioning of Donor Hearts during Transportation with the Organ Care System,” Online, Journal of Cardiac Surgery 34(10): 969-975; Monteagudo Vela, M., Garcia Saez, D., and Simon. A, R. 2018. “Current Approaches in Retrieval and Heart Preservation,” Online, Annals of Cardiothoracic Surgery (Hong Kong, China) 7(1): 67-74; Smulowitz, P. B., Serna, D, L., Beckham, G. E., and Milliken, J, C. 2000, Op cit. above in section 2a, entitled Background. General; Gott, J. P., Brown, W. M. 3rd, Pan-Chih, Dorsey, L. M., Walker, B., and Guyton, R, A. 1992. “Cardioplegia for Heart Transplantation: Unmodified UW [University of Wisconsin] Solution Compared with Stanford Solution,” Journal of Heart and Lung Transplantation (New York, New York) 11(2 Part1): 353-361).

When a prospective donor is in a deep coma from which recovery is impossible and is already present at or close to a medical center allowing a switched-organ technique to be used, ischemia and organ preservation are eliminated and the multifarious measures put forward to contend with the damage done do not apply, nor do any of the numerous topics pertaining to the best means for maintaining organs harvested from a corpse. Even when the graft organ is excised, sustained normothermic perfusion initiated prior to brain death will have substantially ischemia, and therewith, the prospect of reperfusion injury. Once a recipient is identified as a good match, a portion of the preprocedural period is available to accomplish a measure of donor-recipient cross microchimerization.

Currently, however, even when the donor is in the same center as the prospective recipient, organ harvesting may not be initiated until after the donor has died. This works against the effort to maintain the graft organs in an optimal condition for transplantation and leaves instead as a key factor the best way to preserve graft organs taken from a corpse; however, the continuation of life support past death with the aid of an extracorporeal membrane oxygenator and diligent attendance by clinicians expert in preservation can sustain the organs in nearly living condition (see, for example, Kaliyev, R., Lesbekov, T., Bekbossynov, S., Bekbossynova, M., Nurmykhametova, Z., and 8 others 2019. “Comparison of Custodial vs Warm Blood Cardioplegia and Conditioning of Donor Hearts during Transportation with the Organ Care System,” Journal of Cardiac Surgery 34(10): 969-975; Oldani, G., Peloso, A., Slits, F., Gex, Q., Delaune. V., and 8 others 2019. “The Impact of Short-term Machine Perfusion on the Risk of Cancer Recurrence after Rat Liver Transplantation with Donors after Circulatory Death,” Online, PLoS [Public Library of Science] One 14(11): e0224890; Passov. A., Schramko, A., Makisalo, H., Nordin, A., Andersson, S., Pesonen, E., and Ilmakunnas, M. 2019. “Graft Glycocalyx Degradation in Human Liver Transplantation,” Online, PLoS [Public Library of Science] One 14(8): e0221010; Petrenko, A., Carnevale, M., Somov, A., Osorio, J., Rodriguez, J., and 3 others 2019. “Organ Preservation into the 2020s: The Era of Dynamic Intervention,” Transfusion Medicine and Hemotherapy 46(3): 151-172; Roll, G, R. 2019. “An Update on Machine Preservation of the Liver,” Clinical Liver Disease (Hoboken, New Jersey) 14(5): 180-182; Schlegel, A., Muller, X., Kalisvaart, M., Muellhaupt, B., Perera, M. T. P. R., and 4 others 2019. “Outcomes of DCD [donation after circulatory death] Liver Transplantation Using Organs Treated by Hypothermic Oxygenated Perfusion before Implantation,” Journal of Hepatology 70(1): 50-57; Tingle, S., Figueiredo, R. S., Moir, J. A., Goodfellow, M., Talbot, D., and Wilson, C, H. 2019; Op cit., second paragraph above; Boteon, Y, L., Afford, S. C., and Mergental, H. 2018. “Pushing the Limits: Machine Preservation of the Liver as a Tool to Recondition High-Risk Grafts,” Current Transplantation Reports 5(2): 113-120; Burlage, L. C., Tessier, S. N., Etra, J. W., Uygun, K., and Brandacher, G. 2018. “Advances in Machine Perfusion, Organ Preservation, and Cryobiology: Potential Impact on Vascularized Composite Allotransplantation,” Current Opinion in Organ Transplantation 23(5): 561-567, also cited below in section 2b(1)(b11); Fuller, B., Froghi, F., and Davidson, B. 2018. “Organ Preservation Solutions: Linking Pharmacology to Survival for the Donor Organ Pathway,” Current Opinion in Organ Transplantation 23(3): 361-368; Gomez-Dos Santos. V., Ruiz Hernandez, M., and Burgos-Revilla, F. J. 2018. “Hypothermic Machine Perfusion Results in a Marginal Kidney Transplant Programme,” European Urology Focus 4(2): 163-168; Hoste, P., Hoste, E., Ferdinande. P., Vandewoude, K., Vogelaers. D., and 4 others with 21 collaborators 2018. “Development of Key Interventions and Quality Indicators for the Management of An Adult Potential Donor after Brain Death: A RAND [Research and Development Corporation] Modified Delphi Approach,” Online, BioMed Central Health Services Research 18(1): 580; Iskender, I., Cypel, M., Martinu, T., Chen, M., Sakamoto, J., and 8 others 2018. “Effects of Warm versus Cold Ischemic Donor Lung Preservation on the Underlying Mechanisms of Injuries during Ischemia and Reperfusion,” Transplantation 102(5): 760-768; Peng, P., Ding, Z., He, Y., Zhang, J., Wang, X., and Yang, Z. 2018. “Hypothermic Machine Perfusion Versus Static Cold Storage in Deceased Donor Kidney Transplantation: A Systematic Review and Meta-analysis of Randomized Controlled Trials,” Artificial Organs October 3; Salehi, S., Tran, K., and Grayson, W. L. 2018. “Advances in Perfusion Systems for Solid Organ Preservation,” Yale Journal of Biology and Medicine (New Haven, Connecticut) 91(3): 301-312; Steinmeyer, J., Becker, S., Avsar. M., Salman, J., Hoffler, K., and 5 others 2018. Op cit., section 2a; Seetapun, D. and Ross, J. J. 2017. “Eliminating the Organ Transplant Waiting List: The Future with Perfusion Decellularized Organs,” Surgery 161(6): 1474-1478; Tsui, S. S. L. and Oniscu, G, C. 2017. “Extending Normothermic Regional Perfusion to the Thorax in Donors after Circulatory Death,” Current Opinion in Organ Transplantation 22(3): 245-250; Westphal, G. A., Garcia, V. D., Souza, R, L., Franke, C. A., Vieira, K. D., and 29 others 2016. “Guidelines for the Assessment and Acceptance of Potential Brain-dead Organ Donors,” Revista Brasileira de Terapia Intensiva 28(3): 220-255; Kumar, L. 2016, Op cit.; George, S., Thomas, M., Ibrahim, W. H., Abdussalam, A., Chandra, P., Ali, H. S., and Raza. T. 2016. “Somatic Survival and Organ Donation among Brain-dead Patients in the State of Qatar,” Online, BioMed Central Neurology 16(1): 207; Ali, F., Dua, A., and Cronin, D, C. 2015. “Changing Paradigms in Organ Preservation and Resuscitation,” Current Opinion in Organ Transplantation 20(2): 152-158; Rady. M. Y. and Verheijde, J. L. 2013. Brain-dead Patients are Not Cadavers: The Need to Revise the Definition of Death in Muslim Communities,” Hospital Ethics Committee Forum 25(1): 25-45; Rezaee, F., Peppelenbosch, M., and Dashty, M. 2013. “Donor Chimera Model for Tolerance Induction in Transplantation,” Human Immunology 74(5): 550-556; Balfoussia, D., Yerrakalva, D., Hamaoui, K., Papalois, V. 2012. “Advances in Machine Perfusion Graft Viability Assessment in Kidney, Liver, Pancreas, Lung, and Heart Transplant,” Experimental and Clinical Transplantation (Ankara, Turkey) 10(2): 87-100; Westphal, G. A., Caldeira Filho, M., Fiorelli, A., Vieira, K. D., Zaclikevis. V., and 19 others 2012. “Guidelines for Maintenance of Adult Patients with Brain Death and Potential for Multiple Organ Donations: The Task Force of the Brazilian Association of Intensive Medicine the Brazilian Association of Organs Transplantation, and the Transplantation Center of Santa Catarina,” Transplantation Proceedings 44(8): 2260-2267; Guibert, E. E., Petrenko, A. Y., Balaban, C, L., Somov, A. Y., Rodriguez, J. V., and Fuller, B. J. 2011. “Organ Preservation: Current Concepts and New Strategies for the Next Decade,” Transfusion Medicine and Hemotherapy (Basel, Switzerland) 38(2): 125-142; Watson, C. J., Wells, A. C., Roberts, R. J., Akoh, J. A., Friend, P. J., and 5 others 2010. Op cit., section 2a(3); Zaouali, M. A., Ben Abdennebi, H., Padrissa-Altes, S., Mahfoudh-Boussaid, A., and Rosello-Catafau, J. 2010. “Pharmacological Strategies against Cold Ischemia Reperfusion Injury,” Expert Opinion in Pharmacotherapy 2010 11(4): 537-555; Vergoulas, G., Boura. P., and Efstathiadis, G. 2009. “Brain dead Donor Kidneys are Immunologically Active: Is Intervention Justified?,” Hippokratia 13(4): 205-210; Todd, P. M., Jerome, R. N., and Jarquin-Valdivia, A, A. 2007, Op cit. under Background of the Invention. a. General; Gore, S. M., Taylor, R. M., and Wallwork, J. 1991. “Availability of Transplantable Organs from Brain Stem Dead Donors in Intensive Care Units,” Online, British Medical Journal (Clinical Research Edition) 302(6769): 149-153).

“With mechanical ventilation, “brain dead” individuals maintain a wide array of biological functions, including circulation, respiration, wound healing, infection fighting, temperature regulation, secretion of neurohormones, and even gestation of a fetus for up to three months” (Miller, F, G. and Sade, R. M. 2014. “Consequences of the Dead Donor Rule,” Annals of Thoracic Surgery 97(4): 1131-1132; additional references cited below in this section).

Requiring many more years to realize, recent progress in on the production of human embryos through self-organization of stem cells and more conventional tissue engineering may allow the generation of replacement organs locally as needed (see, for example, Amadei, G., Handford, C, E. Qiu. C., de Jonghe, J., Greenfield, H., and 10 others 2022. “Synthetic Embryos Complete Gastrulation to Neurulation and Organogenesis,” Nature Aug. 25, 2022; Collins, S., and Garget. J. 2022. “Synthetic Embryo with Brain and Beating Heart Grown from Muliple Stem Cells,” Online, Science Daily Aug. 25, 2022; Willyard, C. 2022. “Mouse Embryos Grown without Eggs or Sperm: Why and What's Next,” Nature Aug. 25, 2022; Sozen, B., Comwall-Scoones, J., and Zernicka-Goetz, M. 2021. “The Dynamics of Morphogenesis in Stem Cell-based Embryology: Novel Insights for Symmetry Breaking,” Online, Developmental Biology 474:82-90; Camacho-Aguilar, E. and Warmflash, A. 2020. “Insights into Mammalian Morphogen Dynamics from Embryonic Stem Cell Systems,” Online, Current Topics in Developmental Biology 137:279-305; Shahbazi, M. N., Siggia, E. D., and Zernicka-Goetz, M. 2019. “Self-organization of Stem Cells into Embryos: A Window on Early Mammalian Development,” Online, Science 364(6444): 948-951).

As with any donor organ, a good outcome following transplantation is more likely with an ab initio and continuously—not just normothermically perfused after static hypothermic storage-normothermally perfused, nonischemic liver taken from a deceased donor maintained on life support (see, for example, Jadlowiec, C, C. and Taner, T. 2016. “Liver Transplantation: Current Status and Challenges,” World Journal of Gastroenterology 22(18): 4438-4445; Goldaracena, N., Barbas, A. S., and Selzner, M. 2016. “Normothermic and Subnormothermic Ex-vivo Liver Perfusion in Liver Transplantation,” Current Opinion in Organ Transplantation 21(3): 315-321; Hoyer, D. P. and Minor, T. 2016. “Graft Reconditioning before Liver Transplantation,” Visceral Medicine 32(4): 250-256; Ravikumar, R., Leuvenink, H., and Friend, P. J. 2015. “Normothermic Liver Preservation: A New Paradigm?,” Transplant International 28(6): 690-699; Dutkowski, P., Polak, W. G., Muiesan, P., Schlegel, A., Verhoeven, C. J., and 4 others 2015. “First Comparison of Hypothermic Oxygenated PErfusion versus Static Cold Storage of Human Donation after Cardiac Death Liver Transplants: An International-matched Case Analysis,” Annals of Surgery (Philadelphia, Pennsylvania) 262(5): 764-771; Liu, Q., Nassar, A., Farias, K., Buccini, L., Baldwin, W., and 9 others 2014. “Sanguineous Normothermic Machine Perfusion Improves Hemodynamics and Biliary Epithelial Regeneration in Donation after Cardiac Death Porcine Livers,” Liver Transplantation 20(8): 987-999; Oniscu, G. C., Randle, L. V., Muiesan, P., Bourutler, A. J., Currie, I. S., Perera, M. T., Forsythe, J, L., and Watson, C. J. 2014. In Situ Normothermic Regional Perfusion for Controlled Donation after Circulatory Death—The United Kingdom Experience,” American Journal of Transplantation 14(12): 2846-2854; Berendsen, T. A., Izamis, M, L., Xu. H., Liu, Q., Hertl. M., Berthiaume, F., Yarmush, M, L., and Uygun, K. 2011. “Hepatocyte Viability and Adenosine Triphosphate Content Decrease Linearly over Time during Conventional Cold Storage of Rat Liver Grafts,” Transplantation Proceedings 43(5): 1484-1488).

Used apart from the Paragonix, Asporto, or a similar device, special hypothermic solutions such as Somah, Celsior, and University of Wisconsin graft organ preservation hypothermic solution, are less desirable still (Barrou, B., Chatauret, N., Hauet. T., Thuret. R., Kleinclauss. F., and 4 others 2016. “Ischemia-reperfusion. Preservation Solution and Hypothermic Machine Perfusion,” (in French with English abstract at Pubmed), Progres en Urologie 26(15): 964-976; Minasian, S. M., Galagudza, M. M., Dmitriev, Y. V., Karpov, A. A., and Vlasov, T, D. 2015. “Preservation of the Donor Heart: From Basic Science to Clinical Studies,” Interactive Cardiovascular and Thoracic Surgery 20(4): 510-519; Lowalekar, S. K., Cao, H., Lu, X. G., Treanor, P. R., and Thatte, H. S. 2014. “Sub-normothermic Preservation of Donor Hearts for Transplantation Using a Novel Solution, Somah: A Comparative Pre-clinical Study,” Journal of Heart and Lung Transplantation (New York, New York) 33(9): 963-970).

No less true of a liver as compared with the transplantation of any organ, ischemia time, which sudden and metered compound vascular bypass, or sudden switched-organ transplantation eliminate, is inimical to obtaining a favorable outcome (see, for example, Mahboub, P., Bozorgzadeh, A., and Martins, P. N. 2016. “Potential Approaches to Improve the Outcomes of Donation after Cardiac Death Liver Grafts,” Online, World Journal of Transplantation (Pleasanton, California) 6(2): 314-320; Sibulesky, L., Li, M., Hansen, R. N., Dick, A. A., Montenovo, M. I., Rayhill, S. C., Bakthavatsalam, R., and Reyes, J, D. 2016. “Impact of Cold Ischemia Time on Outcomes of Liver Transplantation: A Single Center Experience,” Annals of Transplantation 21:145-151; Matsuno, N., Uchida, K., and Furukawa, H. 2014. “Impact of Machine Perfusion Preservation of Liver Grafts from Donation after Cardiac Death,” Transplantation Proceedings 46(4): 1099-1103; Shapey, I. M. and Muiesan, P. 2013. “Regional Perfusion by Extracorporeal Membrane Oxygenation of Abdominal Organs from Donors after Circulatory Death: A Systematic Review,” Liver Transplantation 19(12): 1292-1303).

Often unexpected, death away from medical facilities where ischemia time is unavoidable demands measures to restore the prospective graft organ such as a liver to a transplantable condition, supporting the discouragement of static cold storage (see, for example, Boteon, Y. L. and Afford, S, C. 2019. “Machine Perfusion of the Liver: Which is the Best Technique to Mitigate Ischaemia-reperfusion Injury?,” Online, World Journal of Transplantation (Pleasanton, California) 9(1): 14-20; Monteagudo Vela. M., Garcia Saez, D., and Simon, A, R. 2018. “Current Approaches in Retrieval and Heart Preservation,” Annals of Cardiothoracic Surgery (Hong Kong, China) 7(1): 67-74; Jayant. K., Reccia, I., Virdis, F., and Shapiro, A. M. J. 2018. “The Role of Normothermic Perfusion in Liver Transplantation (TRANSIT Study): A Systematic Review of Preliminary Studies,” HPB (Hepatic, Pancreatic, and Biliary) Surgery 2018:6360423; Boteon, Y, L., Boteon, A. P., Attard, J., Wallace, L., Bhogal, R. H., Afford, S, C. 2018. “Impact of Machine Perfusion of the Liver on Post-transplant Biliary Complications: A Systematic Review,” Online, World Journal of Transplantation (Pleasanton, California) 8(6): 220-231; Jayant, K., Reccia, I., and Shapiro, A. M. J. 2018. “Normothermic Ex-vivo Liver Perfusion: Where Do We Stand and Where to Reach?,” Expert Reviews of Gastroenterology and Hepatology 12(10): 1045-1058; Laing, R. W., Mergental. H., and Mirza, D, F. 2017. “Normothermic Ex-situ Liver Preservation: The New Gold Standard,” Current Opinion in Organ Transplantation 22(3): 274-280; De Carlis. R., Di Sandro, S., Lauterio, A., Ferla, F., Dell′Acqua, A., Zanierato, M., and De Carlis. L. 2017. “Successful Donation after Cardiac Death Liver Transplants with Prolonged Warm Ischemia Time Using Normothermic Regional Perfusion,” Liver Transplantation 23(2): 166-173; Laing, R. W., Mergental. H., Yap. C., Kirkham, A., Whilku, M., and 12 others 2017. “Viability Testing and Transplantation of Marginal Livers (VITTAL) Using Normothermic Machine Perfusion: Study Protocol for an Open-label. Non-randomised, Prospective, Single-arm Trial,” Online, British Medical Journal Open 7(11): e017733; Vogel. T., Brockmann, J. G., Quaglia, A., Morovat, A., Jassem, W., and 3 others 2017. “The 24-hour Normothermic Machine Perfusion of Discarded Human Liver Grafts,” Liver Transplantation 23(2): 207-220; Mergental. H., Perera, M. T., Laing, R. W., Muiesan, P., Isaac, J. R., and 7 others 2016. “Transplantation of Declined Liver Allografts following Normothermic Ex-situ Evaluation,” American Journal of Transplantation 16(11): 3235-3245; Angelico, R., Perera, M. T., Ravikumar, R., Holroyd, D., Coussios, C., and 7 others 2016. “Normothermic Machine Perfusion of Deceased Donor Liver Grafts is Associated with Improved Postreperfusion Hemodynamics,” Transplantation Direct 2(9): e97; Watson, C. J., Kosmoliaptsis. V., Randle, L. V., Russell, N. K., Griffiths, W. J., Davies, S., Mergental. H., and Butler, A. J. 2016. “Preimplant Normothermic Liver Perfusion of a Suboptimal Liver Donated after Circulatory Death,” American Journal of Transplantation 16(1): 353-357; Matsuno, N., Uchida, K., and Furukawa, H. 2014, Op cit. second paragraph above; Morrissey, P, E. and Monaco, A. P. 2014. “Donation after Circulatory Death: Current Practices, Ongoing Challenges, and Potential Improvements,” Transplantation 97(3): 258-264; additional references cited below in this section).

When the donor has died away from a medical facility so that no opportunity to sustain circulation and body warmth past death was possible and the period since expiration had been relatively brief, a ‘non-heart-beating donor’ heart delivered under the support of a portable warming and pulsatile perfusion device—the Organ Care System®, TransMedics; Andover, Massachusetts)—can be used. A ‘beating heart transplant’ still follows the unavoidable preceding period following circulatory death during which the graft organ did not beat and was subjected to anoxia when the donor is remote, which is the reason for advocating the creation of local deceased donor sustainment centers above in this section.

These centers are intended to support deceased donors in order to maintain all of their transplantable organs commencing prior to death by initiating life support and providing endocrine function, normothermal maintenance, antimicrobial, and so on before circulatory death, that is, before they die, thus completely eliminating pretransplantation anoxia from the outset, not after a period of anoxia, as well as the loss of other functions which degrades until it precludes the usability of a corpse as an organ donor

Commencing in anticipation of impending circulatory death by initiating life support if not already having been begun, hence, sustaining regular pulsatile blood flow, and providing endocrine function, normothermal maintenance, antimicrobial, and so on before death, the usability of the donor organs is not allowed to degrade to the condition found in a corpse. When any of the organs are transplanted, its blood supply and drainage vessels are directly connected to those corresponding in the recipient and the compound vascular organ transfer procedure initiated until the transfer of the organ from the circulatory system of the donor into that of the recipient.

Using the sudden or metered compound vascular bypass, or switched organ, method as shown in FIGS. 15 and 16 to transplant a heart or any other solid organ, the total ischemic time from beginning to end is zero. As schematically depicted in FIGS. 25A and 25B, using vascular servovalves to perform a carotid endarterectomy, or as shown in FIGS. 11 thru 13B to perform an extracardiac transposition of the great vessels, the total ischemic time from beginning to end is zero.

Accordingly, compound vascular bypass solid organ transplantation completely, and not to some lesser degree, eliminates pretransplantation anoxia from the outset to provide graft organs in a critically better condition for transplantation (see Krishnan, A., Kasinpila, P., Wang, H., Ruaengsri. C., Shudo, Y., Jackson, E., and Woo, Y. J. 2023. “First-in-Human Beating-heart Transplant,” Online, Journal of Thoracic and Cardiovascular Surgery (New York, New York) 19:80-85)(see also, Chan, J, L., Kobashigawa, J. A., Reich, H. J., Ramzy, D., Thottam, M. M., and 8 others 2017. “Intermediate Outcomes with Ex-vivo Allograft Perfusion for Heart Transplantation,” Journal of Heart and Lung Transplantation (New York, New York) 36(3): 258-263; Shudo, Y., Hiesinger, W., Oyer, P. E., and Woo, Y. J. 2017. “Operative Technique and Pitfalls in Donor Heart Procurement,” Asian Cardiovascular and Thoracic Annals (Chen Yuan, Singapore) 25(1): 80-82; Fatullayev. J., Samak. M., Sabashnikov, A., Weymann, A., Mohite, P. N., and 6 others 2015. Op cit., above in section 2a, Garcia Saez, D., Zych, B., Sabashnikov, A., Bowles, C. T., De Robertis, F., and 13 others 2014. “Evaluation of the Organ Care System in Heart Transplantation with an Adverse Donor/Recipient Profile,” Annals of Thoracic Surgery 98(6): 2099-2106; additional references cited below in section 2b(1)(b)(b5), entitled Heterotopic Double Heart Metered Compound Vascular Bypass, or Switched-organ Transplantation [and so on]).

It is significant that with a current beating heart transplant, once the donor has been reached, other than the fact that after an interval during which a regular heartbeat had been reinstated to lessen ischemic damage, the surgical technique employed remains still quite traumatic. This factor is probably as, if not more, significant for the outcome than is reducing, albeit, without eliminating, ischemia/reperfusion injury. In a compound vascular bypass heart transplant, both the donor and recipient hearts remain intact throughout. Neither heart is ever incised or otherwise traumatized. Rather, the donor and failed native hearts are eventually removed because orthotopic placement in the middle mediastinal compartment makes this unavoidable.

Continuous transfer from the circulatory system of the donor into the circulatory system of the recipient makes this possible, and this requires vascular valves for a sudden switch emergency operation or vascular servovalves for the much preferred metered switch method which affords time, and if necessary, with the aid of immune tolerance induing drugs for the gradual and mutual acclimation through microchimerization between the immune systems of the recipient and donor.

In a compound vascular bypass heart transplant, there is no interruption in pulsatile arterial flow either before or after transfer of the graft organ directly into the circulatory system of the recipient. When the patient enters into circulatory death, a synchronization circuitry-controlled automatic transfer switch smoothly transfers control of beating from the innate conduction system (cardionector, cardionecteur) to the heart lung or cardiopulmonary bypass machine, which if to be sustained for a term longer than such support remains sufficient, is switched in turn to a membrane oxygenator. When the graft organ is placed orthotopically in the central compartment of the mediastinum, the reverse transfer in control is effectuated. Never stopped from beating, the need for cardioplegia plays no part in the procedure.

Accordingly, what would appear to be an unqualified assertion of uninterrupted coronary perfusion in a beating-heart transplant is valid only for the period following recovery of the body to placement on the support machine, with the transferring the control of circulation from the support machine to the graft organ when positioning it in the recipient. In fact, the authors do make this qualification understood. Otherwise, the operation involves much surgery to create the anastomoses between the graft organ and the circulatory system of the recipient. Essentially, the ‘beating heart transplant’ eliminates the second cardioplegic arrest toward the end of the procedure.

Furthermore, in a compound vascular double heart transplant as addressed below in section 2b(1)(b)(b5), entitled Heterotopic Double Heart Metered Compound Vascular Bypass, or Switched-organ Transplantation [and so on], provided the failing heart of the recipient retains an ejection fraction or absolute stroke volume to a degree that when provided with a second less impaired backup or assistance heart, the two working together in tandem can generate the thrust in pulsatile flow required to sustain normal health, and in a child, development.

FIG. 18B depicts the connections between the corresponding arterial, and FIG. 19B, the venous vessels of the donor and recipient and the solenoid-driven valves and bloodlines used to connect these just before the defective recipient heart is excised and the donor heart placed in the middle compartment of the recipient mediastinum in a sudden switch heart transplant. FIGS. 20C and 21C depict the same in a metered switch heart transplant using vascular servovalves, which in gradually transferring the graft organ from the donor to the recipient, afford an intervening period of reciprocal cross-circulation during which spontaneous cross microchimerization and the administration of immune tolerance-inducing drugs if necessary, prime the donor and recipient to more acclimate to and less prone to reject one another when confronted.

FIG. 22A shows that with the defective native organ, heart or lung, makes no difference, excised, the graft, or donor, organ remains attached to its arterial and venous stumps, vascular servovalves, and the bloodlines which had connected it to the since removed native organ as well as the arterial and venous bloodlines, vascular servovalves and stumps of the since removed native organ. In FIG. 22B, the operator has made certain to leave sufficiently long stumps so that the donor organ can be connected to the native arterial and venous vessels of the recipient circulatory system.

The graft or donor organ is now positioned in place of the since removed native organ so that arterial flow is through the native artery of the recipient, the anastomosis with the donor artery, and the donor vascular servovalve. The bloodline 9 which had connected the corresponding arteries in the donor and recipient is now an arcuate bypass around the anastomosis. If the bloodlines had been too long to implant, that would have been evident to the operator when the procedure was begun to prompt the use of inlet and outlet line switches as shown in FIGS. 32A thru 32F, which allow switching from a long to a short line with no interruption in the flow of blood.

### Creation of Anastomosis Bypasses

Two methods for creating an anastomosis bypass are shown here, the first in compound vascular solid organ transplantation as shown in FIGS. 22A and 22B and the second in using line switches. A third method, applicable to vascular anastomoses in general surgery, is to place solenoid-driven bistable vascular valves to allow switching all flow through either the native or bypass lumen, or if control over the relative proportion into which the flow is to be divided between the two lumina is desired, then vascular servovalves, up- and downstream, that is, in straddling relation to the anastomosis. Automatic control overflow as an isolated function is with a minimal prosthetic disorder control system comprising the valves, bypass, microcontroller chip, druglines to the valves, drug reservoir or reservoirs, and the prescription-program.

Virtually all side-entry devices and confluence conduits incorporate at least one accessory channel into which a drugline connected to an implanted drug reservoir can be inserted to target any fluid drug, solution, or maintenance agent into either the native or bypass lumen. Symptom-relevant sensors signal the controller of a condition or event calling for the transfer of flow to the one pathway or the other in coordination with the release of a drug or another supportive substance. If to serve as one of multiple arms of control in the treatment of multimorbid disease with the object of instating optimal homeostasis across the sum of morbidities, control is by a master control microprocessor.

Switching between lines using paired line switches is evocative of the gradual transfer of flow from the native to the prosthetic segment in placing an inline coupling jacket-secured tie-line type vascular prosthesis; however, a valve allows adjustment at any time after having been placed, and a one time adjustment intrinsic in the procedure for placement cannot function as and does not constitute a valve. While the bypass around the arterial donor-recipient anastomosis could be removed with flow entrusted exclusively to the anastomosis, as made explicit above in this section, barring exceptional circumstances such as an insuperable lack of space, the bypass should be retained as highly advantageous for the continued support of the transplant.

However, system components are sized for prospective patients so that those for a neonate or infant are small enough as not to pose a placement problem. Except to reach around an intervening structure that prevents anastomosis, to not anastomose the stumps and leave them connected through the bloodline would not appear to have much application. The sequence of steps specified above for the arterial vessels at the top of FIG. 22B is the same for those venous at the bottom of the figure.

While urine flow in the upper urinary tract, consisting of the kidneys and ureters, is involuntary with peristalsis continual, in the lower tract, consisting of the bladder, prostate in men, and the urethra, unless kept back beyond a threshold at which a voiding reflex is provoked, it is voluntary and intermittent (see, for example, Hickling, D. R., Sun, T. T., and Wu, X, R. 2015. “Anatomy and Physiology of the Urinary Tract: Relation to Host Defense and Microbial Infection,” Online, Microbiology Spectrum (Washington, D.C.) 3(4): 10.1128/microbiolspec. UTI-0016-2012).

Two methods for placing a side-entry device along a constant flow ductus such as a ureter or blood vessel with little if any extravasation are provided, that using a trepan and water jacket integral to the device described and illustrated in U.S. Pat. No. 11,759,186, entitled Ductus Side-entry Jackets and Prosthetic Disorder Response Systems, to direct a water jet against the hole placed in the side of the ductus thereby restraining flow to the lumen. A second method is essential because as shown in FIGS. 3 and 6, an integral trepan is incompatible with the diversion chute raceway, or channel.

The second method, described above in section 2a, entitled Background of the Invention. General, and below in Section 6, entitled Description of the Preferred Embodiments of the Invention, calls for the operator to use a catheter with a razor sharp trepan ferrule at the leading edge connected to a suction pump to draw out the ostium plug and immediately shift his thumb over the hole. In contrast to a conventional ductus side-entry jacket where the water jacket is fully circumferential, in a vascular side-entry diversion jacket, the water jacket omits the sector occupied by the chute and its drive.

By contrast, even with the Organ Care Systeme®, (TransMedics; Andover, Massachusetts) applied after the first two hours or so after death away from a clinic and harvesting, the donor organ will still have been subjected to a medically significant period of cold storage anoxia (Kounatidis, D., Brozou, V., Anagnostopoulos, D., Pantos, C., Lourbopoulos, A., and Mourouzis, I. 2023. “Donor Heart Preservation: Current Knowledge and the New Era of Machine Perfusion” Online, MDPI [Multidisciplinary Digital Publishing Institute] International Journal of Molecular Sciences (Basel, Switzerland) 24(23): 16693; Bryner, B. S., Schroder, J. N., and Milano, C, A. 2021. “Heart Transplant Advances: Ex vivo Organ-preservation Systems,” Online, JTCVS [Journal of Thoracic and Cardiovascular Surgery] Open (Amsterdam, North Holland Netherlands) 8: 123-127; Schaink. A., Gajic-Veljanoski, O., Wells. D., and Holubowich, C. 2020. “Portable Normothermic Cardiac Perfusion System in Donation after Cardiocirculatory Death: A Health Technology Assessment,” Online, Ontario Health Technology Assessment Series (Toronto Ontario, Canada) 20(3): 1-90; Tolboom, H., Olejničková, V., Reser, D., Rosser, B., Wilhelm, M. J., and 3 others 2016, Op cit.; Steen, S., Paskevicius, A., Liao, Q., and Sjöberg. T. 2016. “Safe Orthotopic Transplantation of Hearts Harvested 24 Hours after Brain Death and Preserved for 24 Hours,” Scandinavian Cardiovascular Journal 50(3): 193-120; Sabashnikov, A., Zeriouh, M., Mohite. P. N., Patil, N. P., Garcia-Saez, D., and 7 others 2016. “Moving Back to the Future: Use of Organ Care System Lung for Lobectomy before Lobar Lung Transplantation,” Medical Science Monitor Basic Research 22:70-74; Zeriouh, M., Sabashnikov, A., Mohite, P. N., Zych, B., Patil, N. P. and 9 others 2016. “Utilization of the Organ Care System for Bilateral Lung Transplantation: Preliminary Results of a Comparative Study,” Interactive Cardiovascular and Thoracic Surgery 23(3): 351-357).

The Department of Cardiothoracic Surgery, Skáne University Hospital, Scania, Sweden and Lund University have developed a portable ventilator and cardiopulmonary support machine (Steen, S., Paskevicius, A., Liao, Q., and Sjöberg. T. 2016, Op. cit.). Another system, capable of operating in the different basic perfusion modes (Langendorff Mode, Pump-Supported Working-Mode, and Passive Afterload Working-Mode) and applying each differentialy has been developed by the University of Toronto (Xin, L., Gellner, B., Ribeiro, R. V. P., Ruggeri, G. M., Banner, D., and 4 others 2017. “A New Multi-mode Perfusion System for Ex Vivo Heart Perfusion Study,” Journal of Medical Systems 42(2): 25). A biventricular perfusion system has been developed by the Ludwig Maximilian University, Munich, Germany (Abicht, J. M., Mayr. T, A. J., Jauch, J., Guethoff, S., Buchholz, S., Reichart, B., and Bauer, A. 2018. “Large-animal Biventricular Working Heart Perfusion System with Low Priming Volume-Comparison between In Vivo and Ex Vivo Cardiac Function,” Thoracic and Cardiovascular Surgeon 66(1): 71-82).

For ex-vivo preservation, normothermic beating with whole blood-based perfusate is superior to hypothermic nonbeating perfusion, much less static cold storage (see, for example, Chew, H. C., Macdonald, P. S., and Dhital, K. K. 2019. “The Donor Heart and Organ Perfusion Technology,” Journal of Thoracic Disease 11 (Supplement 6): S938-S945; Kaliyev. R., Lesbekov, T., Bekbossynov, S., Bekbossynova, M., Nurmykhametova, Z., and 8 others 2019. “Comparison of Custodiol vs Warm Blood Cardioplegia and Conditioning of Donor Hearts during Transportation with the Organ Care System,” Journal of Cardiac Surgery (Hoboken, New Jersey) 34(10): 969-975; White, C. W., Messer, S., Large, S. R., Conway, J., Kim, D. H., and 3 others 2018. “Transplantation of Hearts Donated after Circulatory Death,” Online, Frontiers in Cardiovascular Medicine (Lausanne, Switzerland) 5:8; Van Raemdonck, D., Rega, F., Rex. S., and Neyrinck, A. 2018. “Machine Perfusion of Thoracic Organs,” Journal of Thoracic Disease 10 (Supplement 8): S910-S923; Jing, L., Yao, L., Zhao, M., Peng, L. P., and Liu, M. 2018. “Organ Preservation: From the Past to the Future,” Acta Pharmacologica Sinica 39(5): 845-857; Steinmeyer, J., Becker, S., Avsar, M., Salman, J., Hoffler, K., and 5 others 2018, Op cit., sections 2a and 2a(3); Tsui, S. S. L. and Oniscu, G, C. 2017. “Extending Normothermic Regional Perfusion to the Thorax in Donors after Circulatory Death,” Current Opinion in Organ Transplantation 22(3): 245-250; Church, J. T., Alghanem, F., Deatrick, K. B., Trahanas, J. M., Phillips, J. P., and 6 others 2017. “Normothermic Ex Vivo Heart Perfusion: Effects of Live Animal Blood and Plasma Cross-circulation,” American Society for Artificial Internal Organs Journal 63(6): 766-773; Loor, G., Howard, B. T., Spratt, J. R., Mattison, L. M., Panoskaltsis-Mortari. A., and 6 others 2017. “Prolonged EVLP [ex vivo lung perfusion] Using OCS [Organ Care System] Lung: Cellular and Acellular Perfusates,” Transplantation 101(10): 2303-2311; Leprince, P., Popov, A. F., Simon, A. R., Benk, C., and Siepe, M. 2016. “Ex Vivo Perfusion of the Heart with the Use of the Organ Care System,” European Journal of Cardiothoracic Surgery 49(5): 1318-1320; Macdonald, P. S., Chew, H. C., Connellan, M., and Dhital, K. 2016. “Extracorporeal Heart Perfusion Before Heart Transplantation: The Heart in a Box,” Current Opinion in Organ Transplantation 21(3): 336-342; Dhital, K., Simon, A., and Macdonald, P. 2015. “Ex-vivo Donor Heart Perfusion: Testing the Limits of Cardiac Resilience,” Heart, Lung, and Circulation 24(12): 1191-1192; Iyer. A., Gao, L., Doyle, A., Rao. P., Cropper, J. R., and 11 others 2015. “Normothermic Ex Vivo Perfusion Provides Superior Organ Preservation and Enables Viability Assessment of Hearts from DCD [donation after circulatory death] Donors,” American Journal of Transplantation 15(2): 371-380; Messer, S., Ardehali. A., and Tsui, S. 2015. “Normothermic Donor Heart Perfusion: Current Clinical Experience and the Future,” Transplant International 28(6): 634-642; Stamp, N, L., Shah, A., Vincent. V., Wright, B., Wood, C., and 5 others 2015. “Successful Heart Transplant after Ten Hours Out-of-body Time Using the Trans Medics Organ Care System,” Heart, Lung, and Circulation 24(6): 611-613; Tolboom, H., Makhro, A., Rosser, B. A., Wilhelm, M. J., Bogdanova. A., and Falk, V. 2015. “Recovery of Donor Hearts after Circulatory Death with Normothermic Extracorporeal Machine Perfusion,” European Journal of Cardiothoracic Surgery 47(1): 173-179; White, C. W., Hasanally, D., Mundt. P., Li, Y., Xiang. B., and 1 other 2015. “A Whole Blood-based Perfusate Provides Superior Preservation of Myocardial Function during Ex Vivo Heart Perfusion,” Journal of Heart and Lung Transplantation (New York, New York) 34(1): 113-121; Yang. Y., Lin, H., Wen, Z., Huang, A., Huang, G., Hu, Y., Zhong. Y., and Li, B. 2013. “Keeping Donor Hearts in Completely Beating Status with Normothermic Blood Perfusion for Transplants,” Annals of Thoracic Surgery 95(6): 2028-2034; Kuang, J., Sun, Y., Wang, W., Ke. H., and Ye, H. 2015. “Myocardial Apoptosis and Injury of Donor Hearts Kept in Completely Beating Status with Normothermic Blood Perfusion for Transplants,” International Journal of Clinical and Experimental Medicine 8(4): 5767-5773)(see also, Kuzmiak-Glancy, S., Covian. R., Femnou, A. N., Glancy, B., Jaimes, R. 3rd, and 5 others 2018. “Cardiac Performance is Limited by Oxygen Delivery to the Mitochondria in the Crystalloid-perfused Working Heart,” American Journal of Physiology. Heart and Circulatory Physiology 314(4): H704-H715; Farine, E., Egle, M. U., Boone, A. C., Christensen, S., Carrel, T. P., Tevaearai Stahel, H. T., and Longnus, S. L. 2018. “Development of a Cardiac Loading Device to Monitor Cardiac Function During Ex Vivo Graft Perfusion,” Online, PLoS [Public Library of Science] One 13(4): e0195721; Xin, L., Gellner, B., Ribeiro, R. V. P., Ruggeri, G. M., Banner, D., and 4 others 2017, Op cit. above in this section; Messer, S., Axell, R. G., Colah, S., White, P. A., Ryan, M., and 14 others 2016. “Functional Assessment and Transplantation of the Donor Heart after Circulatory Death,” Journal of Heart and Lung Transplantation (New York, New York) 35(12): 1443-1452; Kuzmiak-Glancy, S., Jaimes. R. 3rd. Wengrowski, A. M., and Kay, M. W. 2015. “Oxygen Demand of Perfused Heart Preparations: How Electromechanical Function and Inadequate Oxygenation Affect Physiology and Optical Measurements,” Experimental Physiology 100(6): 603-616; White, C. W., Dhital, K. K., Iyer, A., Connellan, M., Chew, H. C., Gao, L., and 16 others 2015. “Adult Heart Transplantation with Distant Procurement and Ex-vivo Preservation of Donor Hearts after Circulatory Death: A Case Series,” Lancet 385(9987): 2585-2591).

Where, as preferred, a deceased donor having been kept on life support past death provides a heart maintained with normothermic and heartbeat-circulated blood, and the TransMedics device provides a donor heart taken under this circumstance maintained with normothermic nonheartbeat-circulated pulsatile perfusion, another device, the Paragonix Sherpa Perfusion™ Cardiac Transport System, provides hypothermic pulsatile perfusion (see, for example, Hameed, A. M., Lu, D. B., Patrick, E., Xu. B., Hu, M., and 17 others 2019. “Brief Normothermic Machine Perfusion Rejuvenates Discarded Human Kidneys,” Transplantation Direct 5(11): e502; Michel, S. G., LaMuraglia Ii, G. M., Madariaga, M, L., and Anderson, L. M. 2015. Innovative Cold Storage of Donor Organs Using the Paragonix Sherpa Pak I M Devices,” Heart Lung Vessels 7(3): 246-255; Michel, S. G., La Muraglia, G. M. 2nd, Madariaga, M, L., Titus, J. S., Selig. M. K., and 4 ohers 2015. “Twelve-hour Hypothermic Machine Perfusion for Donor Heart Preservation Leads to Improved Ultrastructural Characteristics Compared to Conventional Cold Storage,” Annals of Transplantation 20:461-468; Michel, S. G., La Muraglia, G. M. 2nd, Madariaga, M, L., Titus, J. S., Selig, M. Ks., and 4 others 2014. “Preservation of Donor Hearts Using Hypothermic Oxygenated Perfusion,” Annals of Transplantation 19:409-416), allowing cellular cold injury (see, for example, Messer, S., Ardehali, A., and Tsui, S. 2015, Op cit.). A functionally similar device, the Asporto Heart Preservation Device, is in development (Rivard, A. L. 2015. “Design and Evolution of the Asporto Heart Preservation Device,” Journal of Extra Corporeal Technology 47(2): 119-124).

When the donor heart must be transported while continuously machine perfused normothermically, the heart is harvested or recovered first. While in a patient expected to recover, extended tracheal intubation often results in tracheal collapse, here the donor is dead, so that intubation can continue over a long interval, no aspect of the insult carried over to the recipient. This includes machine damaged blood, transplantation performed with the blood of the recipient in the organ having displaced that of the donor. Damage to the blood of the donor by the pump of the cardiopulmonary machine to the extent that it would affect the condition of the donor heart prior to removal is averted by periodically replacing the blood; however, due to its hematological and cognitive complications, cardiopulmonary support is unsuitable for extended use.

Under cardiopulmonary support, the blood is damaged through hemolysis; however, with the sudden and metered switch techniques, the blood of the donor having been subjected to support transferred along with the transplant organ is limited to that inside the organ at the instant of switching into the circulatory system of the recipient. Sudden and metered switching are intended to instantly switch the transplanted organ from the circulatory system of the donor to that of the recipient, and in so doing, accomplish continuous, uninterrupted perfusion through the transplant organ throughout the transplantation procedure.

Along with immunological factors, reperfusion injury is a key source of complications in organ transplantation. When as most often occurs, the donor has died and an interval transpires before machine support can be initiated, the transplant organ must be rejuvenated to avert irreversible damage and avoid reperfusion injury. Eliminating ischemia in transplantation renders inconsequential the shortcomings in reperfusion injury therapy, such as with remote preconditioning, which is thwarted by anesthetic state, sex, and age. Reperfusion injury research also pertains to ischemia other than that encountered in solid organ transplantation where ischemia is unavoidable, to include coronary artery bypass grafting, stroke, and heart attack.

Often unexpected death precludes initiating somatic support to keep the deceased donor continuously perfused, and distance prevents the donor and recipient from being brought together promptly. Because many organs must be preserved where the donor had died unexpectedly or away from a clinic, the means of restoring a graft organ after the cessation of perfusion will remain important. However, even when these factors are uninvolved, rather than to seek a method to maintain perfusion and eliminate reperfusion, life support is shut off stopping perfusion, organs harvested, the body left to deteriorate so that irreversible autolysis destroys cells that might have been used for immune tolerance induction had the body only been provided with somatic support, and the graft organ kept in cold storage.

Thereafter, preliminary measures such as hypoxic preconditioning are instituted to decrease ischemic injury, and means for treating the presumably unavoidable injury pursued after such injury has been incurred (see, for example, Boteon, Y. L. and Afford, S, C. 2019. Op cit.; Billah, M., Ridiandries, A., Allahwala, U., Mudaliar, H.1, Dona, A., and 3 others 2019. “Circulating Mediators of Remote Ischemic Preconditioning: Search for the Missing Link between Non-lethal Ischemia and Cardioprotection,” Oncotarget 10(2): 216-244; Bunte, S., Behmenburg, F., Eckelskemper. F., Mohr, F., Stroethoff, M., and 4 others 2019. “Cardioprotection by Humoral Factors Released after Remote Ischemic Preconditioning Depends on Anesthetic Regimen,” Critical Care Medicine 47(3): e250-e255; Cho, Y. J., Nam, K., Kim, T. K., Choi, S. W., Kim, S., Hausenloy, D., and Jeon, Y. 2019. “Sevoflurane, Propofol and Carvedilol Block Myocardial Protection by Limb Remote Ischemic Preconditioning,” Online, MDPI [Multidisciplinary Digital Publishing Institute] International Journal of Molecular Sciences (Basel, Switzerland) 20(2). pii: E269; Alakoski, T., Ulvila, J., Yrjola, R., Vainio, L., Magga. J., and 3 others 2019. “Inhibition of Cardiomyocyte Sprouty 1 Protects from Cardiac Ischemia-reperfusion Injury,” Basic Research in Cardiology 114(2): 7; Wetzel, M, D. and Wenke, J, C. 2019. “Mechanisms by which Hydrogen Sulfide Attenuates Muscle Function following Ischemia-reperfusion Injury: Effects on Akt Signaling, Mitochondrial Function, and Apoptosis,” Journal of Translational Medicine 17(1): 33; Guo, L., Wu, X., Zhang, Y., Wang, F., Li, J., and Zhu, J. 2019. “Protective Effects of Gastrin-releasing Peptide Receptor Antagonist RC- 3095 in an Animal Model of Hepatic Ischemia/Reperfusion Injury,” Hepatology Research January 17; Hu, C., Li, L., Ding. P., Li, L., Ge. X., and 11 others 2018. “Complement Inhibitor CRIg/FH Ameliorates Renal Ischemia Reperfusion Injury via Activation of PI3K/AKT Signaling,” Journal of Immunology 201(12): 3717-3730; Steichen, C., Giraud, S., Bon, D., Barrou, B., Badet, L., and 8 others 2018. “Barriers and Advances in Kidney Preservation,” Online, BioMed Research International 2018:9206257; Minghua, W., Zhijian, G., Chahua, H., Qiang, L., Minxuan, X., and 10 others 2018. “Plasma Exosomes Induced by Remote Ischaemic Preconditioning Attenuate Myocardial Ischaemia/Reperfusion Injury by Transferring miR-24 [microRNA precursor non-coding RNA],” Cell Death and Disease 9(3): 320; Zagrean, A. M., Hermann, D. M., Opris, I., Zagrean, L., and Popa-Wagner, A. 2018. “Multicellular Crosstalk between Exosomes and the Neurovascular Unit After Cerebral Ischemia. Therapeutic Implications,” Online, Frontiers in Neuroscience 12:811; Ravaioli, M., Baldassare, M., Vasuri. F., Pasquinelli, G., Laggetta, M., and 10 others 2018. “Strategies to Restore Adenosine Triphosphate (ATP) Level after More than 20 Hours of Cold Ischemia Time in Human Marginal Kidney Grafts,” Annals of Transplantation 23:34-44; Kaths, J. M., Paul. A., Robinson, L. A., and Selzner, M. 2018. “Ex Vivo Machine Perfusion for Renal Graft Preservation,” Transplantation Reviews (Orlando, Florida) 32(1): 1-9; Chen, J. and Chopp. M. 2018. “Exosome Therapy for Stroke,” Stroke 49(5): 1083-1090; Hamar, M. and Selzner, M. 2018. “Ex-vivo Machine Perfusion for Kidney Preservation,” Current Opinion in Organ Transplantation 23(3): 369-374; Rosenberg, J. H., Werner, J. H., Moulton, M. J., and Agrawal, D. K. 2018. “Current Modalities and Mechanisms Underlying Cardioprotection by Ischemic Conditioning,” Journal of Cardiovascular Translational Research 11(4): 292-307; Maier, H. T., Ashraf, M. I., Denecke, C., Weiss, S., Augustin, F., and 8 others 2018. “Prediction of Delayed Graft Function and Long- term Graft Survival by Serum and Urinary Neutrophil Gelatinase-associated Lipocalin during the Early Postoperative Phase after Kidney Transplantation,” Online, PLoS [Public Library of Science] One 13(1): e0189932; Xu. M., Wang, X., Banan, B., Chirumbole, D, L., Garcia-Aroz, S., and 11 others 2018. “Anti-CD47 Monoclonal Antibody Therapy Reduces Ischemia-reperfusion Injury of Renal Allografts in a Porcine Model of Donation after Cardiac Death,” American Journal of Transplantation 18(4): 855-867; Stiegler, P., Bausys, A., Leber, B., Strupas, K., and Schemmer, P. 2018. “Impact of Melatonin in Solid Organ Transplantation—Is it Time for Clinical Trials? A Comprehensive Review,” Online, MDPI [Multidisciplinary Digital Publishing Institute] International Journal of Molecular Sciences (Basel, Switzerland) 19(11). pii: E3509; Thorenz, A., Derlin, K., Schroder, C., Dressler, L., Vijayan, V., and 16 others 2018. “Enhanced Activation of Interleukin- 10. Heme Oxygenase-1, and AKT in C5aR2-deficient Mice is Associated with Protection from Ischemia Reperfusion Injury-induced Inflammation and Fibrosis,” Kidney International 94(4): 741-755: Wang, X., Xu, M., Jia, J., Zhang, Z., Gaut, J. P., and 4 others 2018. “CD47 Blockade Reduces Ischemia/Reperfusion Injury in Donation after Cardiac Death Rat Kidney Transplantation,” American Journal of Transplantation 18(4): 843-854; Xie, W., Zhou, P., Sun, Y., Meng, X., Dai, Z., Sun, G., and Sun, X. 2018. “Protective Effects and Target Network Analysis of Ginsenoside Rg1 [protopanaxadiol] in Cerebral Ischemia and Reperfusion Injury: A Comprehensive Overview of Experimental Studies,” Cells 7(12). pii: E270; Deng, J., Feng, J., Liu, T., Lu, X., Wang. W., and 5 others 2018. “Beraprost Sodium Preconditioning Prevents Inflammation, Apoptosis, and Autophagy during Hepatic Ischemia-reperfusion Injury in Mice via the P38 [mitogen-activated protein kinases] and JNK [c-Jun N-terminal kinase] Pathways,” Drug Design, Development, and Therapy (Auckland, New Zealand) 12:4067-4082; Zuluaga, M., Gueguen, V., Letourneur, D., and Pavon-Djavid, G. 2018. “Astaxanthin-antioxidant Impact on Excessive Reactive Oxygen Species Generation Induced by Ischemia and Reperfusion Injury,” Chemico-Biological Interactions 279:145-158; Cappuccilli, M., Capelli, I., Comai. G., Cianciolo, G., and La Manna, G. 2018. “Neutrophil Gelatinase-associated Lipocalin as a Biomarker of Allograft Function after Renal Transplantation: Evaluation of the Current Status and Future Insights,” Artificial Organs 42(1): 8-14; Ansari, J., Kaur. G., and Gavins, F. N, E. 2018. “Therapeutic Potential of Annexin Al in Ischemia Reperfusion Injury,” International Journal of Molecular Science 19(4). pii: E1211; Zhao, H., Alam, A., Soo, A. P., George, A. J. T., and Ma, D. 2018. “Ischemia-reperfusion Injury Reduces Long-term Renal Graft Survival: Mechanism and Beyond” EBioMedicine 28:31-42; Szilágyi, A, L., Matrai. P., Hegyi, P., Tuboly, E., Pecz, D., and 10 others 2018. “Compared Efficacy of Preservation Solutions on the Outcome of Liver Transplantation: Meta-analysis,” World Journal of Gastroenterology 24(16): 1812-1824; Thuillier, R. and Hauet. T. 2018. “Impact of Hypothermia and Oxygen Deprivation on the Cytoskeleton in Organ Preservation Models,” Online, BioMed Research International 2018:8926724; Heinen, A., Behmenburg, F., Aytulun, A., Dierkes, M., Zerbin, L., and 7 others 2018. “The Release of Cardioprotective Humoral Factors after Remote Ischemic Preconditioning in Humans is Age- and Sex-dependent,” Journal of Translational Medicine 16(1): 112; Nordling, S., Brannstrom, J., Carlsson, F., Lu, B., Salvaris. E., and 7 others 2018. “Enhanced Protection of the Renal Vascular Endothelium Improves Early Outcome in Kidney Transplantation: Preclinical Investigations in Pig and Mouse,” (Nature Research) Scientific Reports 8(1): 5220; Sun, Q., Zhou, H., Cao, R., Lin, M., Hua, X., and 7 others 2018. “Donation after Brain Death followed by Circulatory Death, a Novel Donation Pattern, Confers Comparable Renal Allograft Outcomes with Donation after Brain Death,” BioMed Central Nephrology 19(1): 164; Giwa. S., Lewis, J. K., Alvarez, L., Langer, R., Roth, A. E., and 37 others 2017. “The Promise of Organ and Tissue Preservation to Transform Medicine,” Nature Biotechnology 35(6): 530-542; Xue. W., Tian, P., Xiang, H., Ding, X., Pan, X., and 8 others 2017. “Outcomes for Primary Kidney Transplantation from Donation after Citizens' Death in China: A Single Center Experience of 367 Cases,” Online, BioMed Central Health Services Research 17(1): 250; Chen, G., Wang. C., Ko, D. S., Qiu, J., Yuan, X., and 4 others 2017. “Comparison of Outcomes of Kidney Transplantation from Donation after Brain Death, Donation after Circulatory Death, and Donation after Brain Death followed by Circulatory Death Donors,” Clinical Transplantation 31(11); Scalea. J. R., Redfield. R. R., Arpali, E., Leverson, G. E., Bennett, R. J., and 6 others 2017. “Does DCD [donation after circulatory death] Donor Time-to-death Affect Recipient Outcomes? Implications of Time-to-death at a High-volume Center in the United States,” American Journal of Transplantation 17(1): 191-200; Na, N., Li, K., Huang, Z., Miao, B., Hu. C., and 3 others 2017. “Posttransplant Outcomes of Kidneys Donated after Brain Death followed by Circulatory Death: A Cohort Study of 128 Chinese Patients,” Transplantation Direct 3(8): e189; Gilbo, N. and Monbaliu, D. 2017. “Temperature and Oxygenation during Organ Preservation: Friends of Foes?,” Current Opinion in Organ Transplantation 22(3): 290-299; von Horn, C. and Minor. T. 2018. “Improved Approach for Normothermic Machine Perfusion of Cold Stored Kidney Grafts,” American Journal of Translational Research 10(6): 1921-1929; Castellano, G., Franzin, R., Stasi, A., Divella, C., Sallustio, F., and 10 others 2018. “Complement Activation during Ischemia/Reperfusion Injury Induces Pericyte-to-Myofibroblast Transdifferentiation Regulating Peritubular Capillary Lumen Reduction through pERK [protein kinase RNA-like endoplasmic reticulum kinase] Signaling,” Online, Frontiers in Immunology (Lausanne, Switzerland) 9:1002; Van Raemdonck, D., Rega, F., Rex. S., and Neyrinck, A. 2018. Op cit. above in this section; Karimian, N. and Yeh, H. 2017, Op cit.; Liao, W. I., Wu. S. Y., Wu, G. C., Pao, H. P., Tang, S. E., Huang, K, L., and Chu, S. J. 2017. “Correction: Wen-I Liao, et al. Ac2-26, an Annex in Al Peptide, Attenuates Ischemia-reperfusion-induced Acute Lung Injury,” International Journal of Molecular Science 18, 1771,” [paper cited follows] Online, MDPI [Multidisciplinary Digital Publishing Institute] International Journal of Molecular Sciences (Basel, Switzerland) 2018 19(2). pii: E526; Veighey, K. and MacAllister, R. 2017. “Ischemic Conditioning in Kidney Transplantation,” Journal of Cardiovascular Pharmacology and Therapeutics 22(4): 330-336; Hu, H. and Zou, C. 2017. “Mesenchymal Stem Cells in Renal Ischemia-reperfusion Injury: Biological and Therapeutic Perspectives,” Current Stem Cell Research and Therapy 12(3): 183-187; Liao, W. I., Wu. S. Y., Wu, G. C., Pao, H. P., Tang. S. E., Huang, K, L., and Chu, S. J. 2017. “Ac2-26, an Annexin Al Peptide, Attenuates Ischemia-reperfusion-induced Acute Lung Injury,” Online, MDPI [Multidisciplinary Digital Publishing Institute] International Journal of Molecular Sciences (Basel, Switzerland) 18(8). pii: E1771; Richards, J. A., Wigmore, S., Anderton, S. M., and Howie, S, E. M. 2017. “NKT [natural killer T] Cells are Important Mediators of Hepatic Ischemia-reperfusion Injury,” Transplant Immunology 45:15-21; Yuan, X., Li, D., Chen, X., Han. C., Xu, L., Huang, T., Dong, Z., and Zhang, M. 2017. “Extracellular Vesicles from Human-induced Pluripotent Stem Cell-derived Mesenchymal Stromal Cells (hiPSC-MSCs) Protect against Renal Ischemia/Reperfusion Injury via Delivering Specificity Protein (SP1) and Transcriptional Activating of Sphingosine Kinase 1 and Inhibiting Necroptosis,” Cell Death and Disease 8(12): 3200; Zhang. Y., Xia, X., Yan, J., Yan, L., Lu, C., and 6 others 2017. “Mesenchymal Stem Cell-derived Angiogenin Promotes Primodial Follicle Survival and Angiogenesis in Transplanted Human Ovarian Tissue,” Reproductive Biology and Endocrinology 15(1): 18; Zimmerman, M. A., Martin, A., Yee, J., Schiller, J., and Hong, J, C. 2017. “Natural Killer T Cells in Liver Ischemia-reperfusion Injury,” Online, MDPI [Multidisciplinary Digital Publishing Institute] Journal of Clinical Medicine (Basel, Switzerland) 6(4). pii: E41; Lobb, I., Jiang, J., Lian, D., Liu, W., Haig. A., and 5 others 2017, Op cit., section 2a; Giannopoulos, G., Vrachatis, D. A., Panagopoulou, V., Vavuranakis, M., Cleman, M. W., and Deftereos, S. 2017. “Remote Ischemic Conditioning and Renal Protection,” Journal of Cardiovascular Pharmacology and Therapeutics 22(4): 321-329; Nilsson, K. F., Sandin, J., Gustafsson, L. E., and Frithiof, R. 2017. “The Novel Nitric Oxide Donor PDNO [1,2-propanediol nitric oxide] Attenuates Ovine Ischemia-Reperfusion Induced Renal Failure,” Intensive Care Medicine Experimental 5(1): 29; Behmenburg, F., Heinen, A., Bruch, L. V., Hollmann, M. W., and Huhn, R. 2017. “Cardioprotection by Remote Ischemic Preconditioning is Blocked in the Aged Rat Heart in Vivo,” Journal of Cardiothoracic and Vascular Anesthesia 31(4): 1223-1226; White, C., Ambrose, E., Müller, A., Hatami, S., Li, Y., and 9 others 2017. “Impact of Reperfusion Calcium and pH on the Resuscitation of Hearts Donated after Circulatory Death,” Annals of Thoracic Surgery 2017 103(1): 122-130; Grenda, R. 2017. “Delayed Graft Function and Its Management in Children,” Pediatric Nephrology (Berlin, Germany) 32(7): 1157-1167; Huang, X. P., Tan, H., Chen, B. Y., and Deng, C. Q. 2017. “Combination of Total Astragalus Extract and Total Panax Notoginseng [Chinese ginseng] Saponins Strengthened the Protective Effects on Brain Damage through Improving Energy Metabolism and Inhibiting Apoptosis after Cerebral Ischemia-reperfusion in Mice,” Chinese Journal of Integrative Medicine 23(6): 445-452; Harrington, S., Williams, S. Otte, V., Barchman, S., Jones, C.4, Ramachandran, K., and Stehno-Bittel. L. 2017. “Improved Yield of Canine Islet Isolation from Deceased Donors,” Online, BioMed Central Veterinary Research 13(1): 264; Hamaoui, K., Gowers. S., Boutelle, M., Cook, T. H., Hanna, G., and 4 others 2016. “Organ Pretreatment with Cytotopic Endothelial Localizing Peptides to Ameliorate Microvascular Thrombosis and Perfusion Deficits in Ex Vivo Renal Hemoreperfusion Models,” Transplantation 100(12): e128-e139; Hamaoui, K., Gowers. S., Damji, S., Rogers, M., Leong, C, L., and 4 others 2016. “Rapid Sampling Microdialysis as a Novel Tool for Parenchyma Assessment During Static Cold Storage and Hypothermic Machine Perfusion in a Translational Ex Vivo Porcine Kidney Model,” Journal of Surgical Research 200(1): 332-345: Xiao, Z., Banan, B., Xu. M., Jia. J., Manning, P. T., and 7 others 2016. “Attenuation of Ischemia-reperfusion Injury and Improvement of Survival in Recipients of Steatotic Rat Livers Using CD47 Monoclonal Antibody,” Transplantation 100(7): 1480-1489; Tolboom, H., Olejnickova, V., Reser, D., Rosser, B., Wilhelm, M. J., and 3 others 2016. “Moderate Hypothermia During Ex Vivo Machine Perfusion Promotes Recovery of Hearts Donated after Cardiocirculatory Death,” European Journal of Cardiothoracic Surgery 49(1): 25-31; Saat. T. C., van den Akker, E. K., IJzermans, J. N., Dor, F. J., and de Bruin, R. W. 2016. “Improving the Outcome of Kidney Transplantation by Ameliorating Renal Ischemia Reperfusion Injury: Lost in Translation?,” Journal of Translational Medicine 14:20; Hoyer, D. P. and Minor, T. 2016, Op cit.; White, C. W., Ambrose, E., Müller, A., Li, Y., Le, H., and 8 others 2016. “Avoidance of Profound Hypothermia During Initial Reperfusion Improves the Functional Recovery of Hearts Donated after Circulatory Death,” American Journal of Transplantation 16(3): 773-782; Pezeshgi, A., Abedi Azar, S., Ghasemi, H., Kamali, K., Esmaeilzadeh, A., and 4 others 2016. “Role of Plasma Neutrophil Gelatinase-associated Lipocalin as an Emerging Biomarker of Acute Renal Failure following Kidney Transplantation and Its Correlation with Plasma Creatinine,” Journal of Renal Injury Prevention 5(2): 98-103; Heusch, G. and Rassaf. T. 2016. “Time to Give Up on Cardioprotection? A Critical Appraisal of Clinical Studies on Ischemic Pre-, Post-, and Remote Conditioning,” Circulation Research 119(5): 676-695; Farine, E., Niederberge, P., Wyss, R. K., Mendez-Carmona, N., Gahl. B., and 4 others 2016. “Controlled Reperfusion Strategies Improve Cardiac Hemodynamic Recovery after Warm Global Ischemia in an Isolated, Working Rat Heart Model of Donation after Circulatory Death (DCD),” Online, Frontiers in Physiology 7:543 eCollection 2016; Ravingerova, T., Farkasova, V., Griecsova, L., Carnicka, S., Murarikova, M., and 6 others 2016. “Remote Preconditioning as a Novel “Conditioning” Approach to Repair the Broken Heart: Potential Mechanisms and Clinical Applications,” Physiological Research 65 Supplement 1: S55-S64; Lobb. I., Davison, M., Carter, D., Liu. W., Haig. A., Gunaratnam, L., and Sener, A. 2015, Op cit., section 2a; Huang, X. P., Ding, H., Lu, J. D., Tang, Y. H., Deng, B. X., and Deng, C. Q. 2015. “Effects of the Combination of the Main Active Components of Astragalus and Panax Notoginseng [Chinese ginseng] on Inflammation and Apoptosis of Nerve Cell after Cerebral Ischemia-Reperfusion,” American Journal of Chinese Medicine 43(7): 1419-1438; Chen, F. and Date, H. 2015. “Update on Ischemia-reperfusion Injury in Lung Transplantation,” Current Opinion in Organ Transplantation 20(5): 515-520; Iliodromitis, E. K., Cohen, M. V., Dagres, N., Andreadou, I., Kremastinos, D. T., and Downey, J. M. 2015. “What is Wrong with Cardiac Conditioning? We May be Shooting at Moving Targets,” Journal of Cardiovascular Pharmacology and Therapeutics 20(4): 357-369; Li, J., Wang, F., Xia. Y., Dai, W., Chen, K., and 10 others 2015. “Astaxanthin Pretreatment Attenuates Hepatic Ischemia Reperfusion-induced Apoptosis and Autophagy via the ROS/MAPK [reactive oxygen species/mitogen-activated protein kinase] Pathway in Mice,” Marine Drugs 2015 13(6): 3368-3387; Ambrose, E., Müller, A., Li, Y., Le, H., Hiebert, B., and 7 others 2015. “Assessment of Donor Heart Viability During Ex Vivo Heart Perfusion,” Canadian Journal of Physiology and Pharmacology 93(10): 893-901; Salvadori, M., Rosso, G., and Bertoni, E. 2015. “Update on Ischemia-reperfusion Injury in Kidney Transplantation: Pathogenesis and Treatment,” Online, World Journal of Transplantation (Pleasanton, California) 5(2): 52-67; O'Neill, S., Gallagher, K., Hughes, J., Wigmore, S., Ross, J. A., and Harrison, E. M. 2015. “Challenges in Early Clinical Drug Development for Ischemia-reperfusion Injury in Kidney Transplantation,” Expert Opinion on Drug Discovery 10(7): 753-762; Hsu, H. H., Wu. S. Y., Tang, S. E., Wu, G. C., Li, M. H., Huang, K, L., and Chu, S. J. 2015. “Protection against Reperfusion Lung Injury via Aborgating [sic abrogating] Multiple Signaling Cascades by Trichostatin A,” International Immunopharmacology 25(2): 267-275; Rowart. P., Erpicum, P., Detry. O., Weekers, L., Gregoire. C., and 5 others 2015. “Mesenchymal Stromal Cell Therapy in Ischemia/Reperfusion Injury,” Journal of Immunology Research 2015: 602597; Tolboom, H., Makhro, A., Rosser, B. A., Wilhelm, M. J., Bogdanova, A., and Falk, V. 2015. “Recovery of Donor Hearts after Circulatory Death with Normothermic Extracorporeal Machine Perfusion,” European Journal of Cardiothoracic Surgery 47(1): 173-179; Dhital, K. K., White, C. W., Iyer. A., Connellan. M., Chew, H. C., Gao, L., and 16 others 2015. Op cit. above in this section; Fatullayev, J., Samak. M., Sabashnikov, A., Weymann, A., Mohite, P. N., and 6 others 2015. Op cit., above in section 2a: Requião-Moura, L. R., Durão Junior Mde. S., Matos, A. C., and Pacheco-Silva, A. 2015. “Ischemia and Reperfusion injury in Renal Transplantation: Hemodynamic and Immunological Paradigms,” Einstein (Sao Paulo, Brazil) 13(1): 129-135; Menke, J., Sollinger, D., Schamberger, B., Heemann, U., and Lutz, J. 2014. “The Effect of Ischemia/Reperfusion on the Kidney Graft,” Current Opinion in Organ Transplantation 19(4): 395-400; Danobeitia, J. S., Djamali. A., and Fernandez, L, A. 2014. “The Role of Complement in the Pathogenesis of Renal Ischemia-reperfusion injury and Fibrosis,” Fibrogenesis and Tissue Repair 7:16; Branchereau, J. and Barrou, B. 2014. “Pharmacological Strategy Designed to Limit Ischemia-Reperfusion Injury in Brain dead Donor Kidneys,” Progrès en Urologie (Paris, France) 2014 24 Supplement 1: S26-S30; Lin, Y., Manning, P. T., Jia. J., Gaut, J. P., Xiao, Z., and 7 others 2014. “CD47 Blockade Reduces Ischemia-reperfusion Injury and Improves Outcomes in a Rat Kidney Transplant Model,” Transplantation 98(4): 394-401; Zhang, C., Zheng, L., Li, L., Wang, L., Li, L., and 6 others 2014. “Rapamycin Protects Kidney against Ischemia Reperfusion Injury through Recruitment of NKT [natural killer T thymus-derived lymphocyte] Cells,” Journal of Translational Medicine 12:224; Asif. S., Sedigh, A., Nordstrom, J., Brandhorst, H., Jorns. C., and 9 others 2012. “Oxygen-charged HTK-F6H8 [histidine-tryptophan-ketoglutarate perfluorohexyloctane] Emulsion Reduces Ischemia-Reperfusion Injury in Kidneys from Brain-dead Pigs,” Journal of Surgical Research 178(2): 959-967; Ozaki. K. S., Kimura, S., Nalesnik, M. A., Sico, R. M., Zhang. M., and 4 others 2012. “The Loss of Renal Dendritic Cells and Activation of Host Adaptive Immunity are Long-term Effects of Ischemia/Reperfusion Injury following Syngeneic Kidney Transplantation,” Kidney International 81(10): 1015-1025; Niclauss, N., Wojtusciszyn, A., Morel, P., Demuylder-Mischler, S., Brault C., and 6 others 2012. “Comparative Impact on Islet Isolation and Transplant Outcome of the Preservation Solutions Institut Georges Lopez-1. University of Wisconsin, and Celsior,” Transplantation 93(7): 703-708; O'Callaghan, J. M., Knight, S. R., Morgan, R. D., and Morris, P. J. 2012. “Preservation Solutions for Static Cold Storage of Kidney Allografts: A Systematic Review and Meta-analysis,” American Journal of Transplantation 12(4): 896-906; Yates, P. J., Hosgood, S. A., and Nicholson, M. L. 2012. “A Biphasic Response to Nitric Oxide Donation in an Ex Vivo Model of Donation after Cardiac Death Renal Transplantation,” Journal of Surgical Research 175(2): 316-321; Dornbierer, M., Stadelmann, M., Sourdon, J., Gahl, B., Cook, S., and 3 others 2012. “Early Reperfusion Hemodynamics Predict Recovery in Rat Hearts: A Potential Approach towards Evaluating Cardiac Grafts from Non-heart-beating Donors,” Online, PLoS [Public Library of Science] One 7(8): e43642; Siedlecki, A., Irish, W., and Brennan, D, C. 2011. “Delayed Graft Function in the Kidney Transplant,” American Journal of Transplantation 11(11): 2279-2296; Gdynia, H. J., Kühnlein, P., Ludolph, A. C., and Huber, R. 2008. “Connective Tissue Disorders in Dissections of the Carotid or Vertebral Arteries,” Journal of Clinical Neuroscience 15(5): 489-494; Schoen, M., Rotter, R., Gierer, P., Gradl, G., Strauss, U., and 3 others 2007. “Ischemic Preconditioning Prevents Skeletal Muscle Tissue Injury, but Not Nerve Lesion upon Tourniquet-induced Ischemia,” Journal of Trauma 63(4): 788-797; Fahy, G. M., Wowk. B., and Wu, J. 2006. “Cryopreservation of Complex Systems: The Missing Link in the Regenerative Medicine Supply Chain,” Rejuvenation Research 9(2): 279-291; Hausenloy, D. J. and Yellon, D. M. 2006. “Survival Kinases in Ischemic Preconditioning and Postconditioning,” Cardiovascular Research 70(2): 240- 253; Hausenloy, D. J., Tsang. A., and Yellon, D. M. 2005. “The Reperfusion Injury Salvage Kinase Pathway: A Common Target for Both Ischemic Preconditioning and Postconditioning,” Trends in Cardiovascular Medicine 15(2): 69-75).

Following brain death, the heart, activated internally by the cardionector, or conduction system of the heart, continues to beat until circulatory death ensues, typically, about a week later. However, that the pulse is not stable following brain death usually results in little difference in outcome between a renal graft whether harvested after brain or circulatory death (Sun, Q., Zhou, H., Cao. R., Lin, M., Hua, X., and 7 others 2018. Op cit.), and in one exceptional instance, a woman who had been brain dead for 78 days gave birth to a healthy baby girl (Kinoshita, Y., Kamohara, H., Kotera, A., Sagishima, K., Tashiro, T., and Niimori, D. 2014, Op cit. above under 1. Background of the Invention. a. General).

Some post-transplantation chemical monitoring and therapy will remain valid regardless of the method used to preserve the organ prior to transplantation (see, for example, Catic-Dordević. A., Cvetković. T., Stefanović, N., and Veličković-Radovanović, R. 2017. “Current Biochemical Monitoring and Risk Management of Immunosuppressant Therapy after Transplantation,” Journal of Medical Biochemistry 36(1): 1-7; Webster, A. C., Wu. S., Tallapragada, K., Park, M. Y., Chapman, J. R., and Carr, S. J. 2017. “Polyclonal and Monoclonal Antibodies for Treating Acute Rejection Episodes in Kidney Transplant Recipients,” Cochrane Database of Systematic Reviews 7: CD004756; Staatz, C, E. and Tett, S, E. 2014. “Pharmacology and Toxicology of Mycophenolate in Organ Transplant Recipients: An Update,” Archives of Toxicology 88(7): 1351-1389; Penninga, L., Moller, C. H., Gustafsson, F., Gluud, C., and Steinbruchel, D, A. 2013. “Immunosuppressant T-cell Antibody Induction for Heart Transplant Recipients,” Cochrane Database of Systematic Reviews(12): CD008842).

In any transplantation situation, but especially with sudden and metered switching, both recipient and donor are ideally located in the same tertiary or quaternary center, whenever possible, with the donor sustained on extracorporeal membrane oxygenation life support. Ideally, a deceased donor sustainment center as addressed above in section 2a, entitled Background of the Invention. General, specialized to preserve deceased donors in a living a condition as possible will be available. If the centers are distant, the replacement heart is transported at body temperature without interruption in perfusion by means specified below.

The donor without brainstem as well as cerebral activity is then kept on cardiopulmonary support over a relatively brief interval less likely to result in significant hemolysis. Longer intervals are covered using extracorporeal membrane oxygenation, which using larger cannulae, is less damaging to blood cells. Using wider bore cannulae, extracorporeal membrane oxygenation may be continued over a longer period than can cardiopulmonary support. However, extracorporeal membrane oxygenation also carries risks, to include problem bleeding, thrombocytopenia, subarachnoid hemorrhage, and others.

For the purpose of sustaining the transplantability of a deceased donor's organs, extracorporeal membrane oxygenation is superior, both because it can remain in use over a longer period, and because in such a donor the complications it causes are relatively little material for the purpose of harvesting most of his organs (see, for example, McRae, K. and de Perrot, M. 2018. “Principles and Indications of Extracorporeal Life Support in General Thoracic Surgery,” Journal of Thoracic Disease 10 (Supplement 8): S931-S946; Kłosiewicz, T., Puslecki, M., Zieliński, M., Mandecki, M., Ligowski, M., and 9 others 2017. “The Role of Extracorporeal Membrane Oxygenation in Patients after Irreversible Cardiac Arrest as Potential Organ Donors,” Kardiochirurgia i Torakochirurgia Polska [Polish Journal of Cardiothoracic Surgery] 14(4): 253-257; Baran, D, A. 2017. “Extracorporeal Membrane Oxygenation (ECMO) and the Critical Cardiac Patient,” Current Transplantation Reports 4(3): 218-225; Kiefer, J. and Freundlich, R, E. 2017. “Confounding Variables Impacting the Association between Duration of Veno-arterial Extracorporeal Life Support and Mortality,” Critical Care (London, England) 21(1): 203; Smith, M., Vukomanovic, A., Brodie, D., Thiagarajan, R., Rycus, P., and Buscher, H. 2017. “Duration of Veno-arterial Extracorporeal Life Support (VA ECMO) and Outcome: An Analysis of the Extracorporeal Life Support Organization (ELSO) Registry,” Critical Care (London, England) 21(1): 45; Xie. A., Lo. P., Yan, T. D., and Forrest, P. 2017. “Neurologic Complications of Extracorporeal Membrane Oxygenation: A Review,” Journal of Cardiothoracic and Vascular Anesthesia 31(5): 1836-1846; Lorusso, R., Gelsomino, S., Parise, O., Di Mauro, M., Barili, F., and 13 others 2017. “Neurologic Injury in Adults Supported with Veno-Venous Extracorporeal Membrane Oxygenation for Respiratory Failure: Findings from the Extracorporeal Life Support Organization Database,” Critical Care Medicine 45(8): 1389-1397; Li, P., Dong, N., Zhao, Y., and Gao. S. 2016. “Successful Extracorporeal Membrane Oxygenation (ECMO) Support in Two Pediatric Heart Transplant Patients with Extreme Donor/Recipient Size Mismatch,” Journal of Thoracic Disease 8(6): 1329-1332; Trudzinski, F. C., Minko, P., Rapp. D., Fahndrich, S., Haake, H., and 12 others 2016. “Runtime and aPTT [activated partial thromboplastin time] Predict Venous Thrombosis and Thromboembolism in Patients on Extracorporeal Membrane Oxygenation: A Retrospective Analysis,” Annals of Intensive Care 6(1): 66; Balsorano, P., Ciapetti, M., Cianchi, G., Bonizzoli, M., Migliaccio, M, L., and 3 others 2015. “Extracorporeal Life Support and Multiorgan Donation in a Severe Polytrauma Patient: A Case Report,” International Journal of Surgery Case Reports 9:109-111; Lee, H., Cho, Y. H., Sung, K., Yang, J. H., Chung, C. R., Jeon, K., and Suh, G. Y. 2015. “The Use of Extracorporeal Circulation in Suspected Brain dead Organ Donors with Cardiopulmonary Collapse,” Journal of Korean Medical Science 30(12): 1911-1914; Carter, T., Bodzin, A. S., Hirose, H., West. S., Hasz, R., Maley, W. R., and Cavarocchi, N, C. 2014. “Outcome of Organs Procured from Donors on Extracorporeal Membrane Oxygenation Support: An Analysis of Kidney and Liver Allograft Data,” Clinical Transplantation 28(7): 816-820; Hsieh, C. E., Lin, H. C., Tsui, Y. C., Lin, P. Y., Lin, K. H., Chang, Y. Y., and Chen, Y. L. 2011. “Extracorporeal Membrane Oxygenation Support in Potential Organ Donors for Brain Death Determination,” Transplantation Proceedings 43(7): 2495-2498).

If the donor is not in the same center but distant and cannot be expeditiously transported while maintained on machine support, the use of cardioplegia under chilled conditions may be avoided through the use instead of continuous perfusion of the excised heart with warmed blood during transport. Such machines do provide the ability to rehabilitate impaired graft organs during ex vivo perfusion. This is accomplished with the aid of a machine such as the Organ Care System® (TransMedics, Incorporated, Andover, Massachusetts) (see for example, Stamp, N, L., Shah, A., Vincent, V., Wright. B., Wood, C., and 5 others 2015. “Successful Heart Transplant after Ten Hours Out-of-body Time Using the TransMedics Organ Care System,” Heart, Lung, and Circulation 24(6): 611-613; Dhital, K. K., Iyer, A., Connellan, M., Chew, H. C., Gao, L., and 16 others 2015, Op cit., section 2. Background, A. General, and in this section; Dhital, K., Simon, A., and Macdonald, P. 2015. Op cit., this section; Popov, A.,F., Garcia Saez, D., Sabashnikov, A., Patil, N. P., Zeriouh, M., and 9 others 2015. “Utilization of the Organ Care System—A Game-changer in Combating Donor Organ Shortage,” Medical Science Monitor Basic Research 21:29-32; Fatullayev, J., Samak, M., Sabashnikov, A., Weymann, A., Mohite, P. N., and 6 others 2015, Op cit.; above in section 2a: Iyer, A., Gao, L., Doyle, A., Rao, P., Cropper, J. R., and 11 others 2015. “Normothermic Ex Vivo Perfusion Provides Superior Organ Preservation and Enables Viability Assessment of Hearts from DCD [donation after circulatory death] Donors,” American Journal of Transplantation 15(2): 371-380; Ardehali, A., Esmailian, F., Deng, M., Soltesz, E., Hsich E., with 7 others and 6 collaborators 2015. “Ex-vivo Perfusion of Donor Hearts for Human Heart Transplantation (PROCEED II): A Prospective, Open-label, Multicentre, Randomised Non-inferiority Trial,” Lancet 385(9987): 2577-2584; Koerner, M. M., Ghodsizad, A., Schulz, U., El Banayosy, A., Koerfer, R., and Tenderich, G. 2014. “Normothermic Ex Vivo Allograft Blood Perfusion in Clinical Heart Transplantation,” Heart Surgery Forum 17(3): E141-E145; Ghodsizad, A., Bordel. V., Ungerer, M., Karck, M., Bekeredjian, R., and Ruhparwar, A. 2012. “Ex Vivo Coronary Angiography of a Donor Heart in the Organ Care System” Heart Surgery Forum 15(3): E161-E163).

However, using the means described herein for a solid organ transplant avoids ischemia entirely, which circumstance in itself eliminates ischemia reperfusion injury as one of the two main causes for rejection, leaving the induction of immune tolerance to receive greater attention, improves critically the prospects for a favorable outcome, while affording a factor of liberalization as to donor age, and additionally affords a measure of improved matching periprocedurally (see, for example, Kim. I. C., Youn, J. C., and Kobashigawa, J, A. 2018, Op cit. above in section 2a; Kittleson, M. M. and Kobashigawa, J, A. 2017. “Cardiac Transplantation: Current Outcomes and Contemporary Controversies,” Journal of the American College of Cardiology. Heart Failure 5(12): 857-868; Khalifian, S., Brazio. P. S., Mohan, R., Shaffer, C., Brandacher, G., Barth, R. N., and Rodriguez, E, D. 2014. “Facial Transplantation: The First 9 Years,” Lancet (London, England) 384(9960): 2153-2163; Russo, M. J., Iribarne, A., Hong, K. N., Ramlawi. B., Chen, J. M., Takayama, H., Mancini, D. M., and Naka, Y. 2010. “Factors Associated with Primary Graft Failure after Heart Transplantation,” Transplantation 90(4): 444-450; Russo, M. J., Chen, J. M., Sorabella, R. A., Martens, T. P., Garrido, M., and 10 others 2007. “The Effect of Ischemic Time on Survival after Heart Transplantation Varies by Donor Age: An Analysis of the United Network for Organ Sharing Database,” Journal of Thoracic and Cardiovascular Surgery 133(2): 554-559; Parolari, A., Rubini, P., Cannata, A., Bonati, L., Alamanni, F., Tremoli, E., and Biglioli, P. 2002. “Endothelial Damage during Myocardial Preservation and Storage,” Annals of Thoracic Surgery 73(2): 682-690). The greater length of the lines vis a vis those innate is compensated for by narrowing the caliber of these to maintain pressure consistency.

While placement of an implanted automatic disorder response system and transplantation using either the sudden or metered switch technique upon primary or initial transplantation of a solid organ or gland should significantly improve outcomes, conventional therapeutic support generally allows sustainment for about twelve years. The literature inconsistent on the sustainability of a retransplanted organ, in general, a younger patient will require retransplantation of the heart. Performed conventionally rather by use of a switch technique, and without the support of an implanted automatic response system, a retransplant should last for at most an equal, but more likely shorter period, with increased transplant-related morbidities and shorter late-term survival.

Retransplantation within a year of the primary operation has a prognosis poor enough to indicate that a second transplantation would be futile; however, where previous transplants have remained competent for years, retransplantation is usually successful. Broadly, survival after the first month bodes favorably for primary transplantation and retransplantation, the relative durability of the latter not definitively established by the literature. While allograft support therapy is undergoing improvement, historically, primary graft failure is taken as a negative indication for retransplantation. Avoiding endothelial degradation and reperfusion injury, and achieving the best match within the restricted time available, switch transplantation should extend the viable life of both primary, and if necessary, retransplants.

More recently, pediatric cardiac outcomes have achieved improved survival; however, neurodevelopmental consequences of delays in establishing normal circulation remain a problem. Given that younger patients will require to be retransplanted, the immediate support provided by the means to be described should have maintained the body in a more robust condition to receive another replacement organ, likely extending the term over which the original transplant will remain competent, and in an adult, more likely to eliminate the need for a retransplantation.

High mutual dependency of one organ upon another such that disease in either will lead to disease in the other-usually all the organs and tissues of the body-prompts the simultaneous or successive replacement of both. That either or both have been impaired and the one then transplanted creates further impairment. Because it can monitor and directly target drugs to exclusively treat either organ within the context of attaining the optimal homeostasis for the patient as a whole, that transplantation and therapeutic support of one solid organ vitally interdependent with another as to bode the degradation of both unless both are transplanted together as seen in the cardiorenal, hepatorenal, cardiohepatic, and other multiple cardiopulmonary syndromes, is effectively if not really, a condition of comorbidity.

Continuously vigilant and prepared to react immediately, a fully implanted, hence, ambulatory automatic prosthetic disorder response system is uniquely situated and constituted to treat complex multimorbidity. (see, for example, Ayyad, A. and Al-Horani, R, A. 2023. “Terlipressin for the Prevention and Treatment of Renal Decline in Hepatorenal Syndrome: A Drug Profile,” Online, Gastroenterology Insights (Pavia, Italy) 14(4): 420-430; Erstad, B. L. 2023. “Hepatorenal Syndrome with Acute Kidney Injury: Diagnosis and Medical Management,” Annals of Pharmacotherapy (Thousand Oaks, California) June 4:10600280231177698; Roy, P., Minhaz, N., Shah-Riar, P., Simona, S. Y., Tasha. T., and 8 others 2023. “A Comprehensive Systematic Review of the Latest Management Strategies for Hepatorenal Syndrome: A Complicated Syndrome to Tackle,” Online, Cureus (Palo Alto, California) 15(8): e43073; Khemichian, S., Francoz, C., and Nadim, M. K. 2021. “Advances in Management of Hepatorenal Syndrome,” Current Opinion in Nephrology and Hypertension (London, England) 30(5): 501-506; Subedi, A., Suresh Kumar, V, C. Sharma Subedi, A., and Sapkota, B. 2021. “A Review of Hepatorenal Syndrome,” Online, Cureus (Palo Alto, California) 13(7): e16084; Deep. A., Saxena, R., and Jose, B. 2019. “Acute Kidney Injury in Children with Chronic Liver Disease,” Pediatric Nephrology 34(1): 45-59; Francoz, C., Durand, F., Kahn, J. A., Genyk, Y. S., and Nadim, M. K. 2019. “Hepatorenal Syndrome,” Online, Clinical Journal of the American Society of Nephrology (Hagerstown, Maryland) 14(5): 774-781; Rucker, A. J., Anderson, K, L., Mulvihill, M. S., Yerokun, B. A., Barbas, A. S., and Hartwig, M, G. 2018. “Simultaneous versus Sequential Heart-Liver Transplantation: Ideal Strategies for Organ Allocation,” Transplantation Direct 5(1): e415; Hussain, S. M. and Sureshkumar, K. K. 2018. “Refining the Role of Simultaneous Liver Kidney Transplantation,” Journal of Clinical and Translational Hepatology 6(3): 289-295; Taiwo, A. A., Khush, K. K., Stedman, M. R., Zheng. Y., and Tan, J, C. 2018. “Longitudinal Changes in Kidney Function following Heart Transplantation: Stanford Experience,” Clinical Transplantation 32(11): e13414; Celik, N., Mazariegos, G. V., Soltys, K., Rudolph, J. A., Shi, Y., Bond, G. J., Sindhi, R., and Ganoza, A. 2018. “Pediatric Intestinal Transplantation,” Gastroenterology Clinics of North America 47(2): 355-368; Freischlag, K. W., Messina, J., Ezekian, B., Mulvihill, M. S., Barbas, A., and 5 others 2018. “Single-center Long-term Analysis of Combined Liver-Lung Transplant Outcomes,” Transplantation Direct 4(5): e349; Yadav, K., Serrano, O. K., Peterson, K. J., Pruett, T, L., Kandaswamy, R., and 5 others 2018. “The Liver Recipient with Acute Renal Dysfunction: A Single Institution Evaluation of the Simultaneous Liver-Kidney Transplant Candidate,” Clinical Transplantation 32(1); Modi, R. M., Tumin, D., Kruger, A. J., Beal, E. W., Hayes, D. Jr., and 6 others 2018. “Effect of Transplant Center Volume on Post-transplant Survival in Patients Listed for Simultaneous Liver and Kidney Transplantation,” World Journal of Hepatology 10(1): 134-141; Nagai, S., Safwan, M., Collins, K., Schilke, R. E., Rizzari. M., and 5 others 2018. “Liver Alone or Simultaneous Liver-Kidney Transplant? Pretransplant Chronic Kidney Disease and Post-transplant Outcome—A Retrospective Study,” Transplant International 2018 May 2; Miles, C. D., Westphal, S., Liapakis, A., and Formica, R. 2018. “Simultaneous Liver-Kidney Transplantation: Impact on Liver Transplant Patients and the Kidney Transplant Waiting List,” Current Transplantation Reports 5(1): 1-6; Singal, A. K., Ong. S., Satpathy, S., Kamath, P. S., and Wiesner, R, H. 2018. “Simultaneous Liver Kidney Transplantation” Transplant International December 10; Grant, L., Tujios, S., and Singal, A, G. 2018. “Outcomes of Simultaneous Liver-Kidney Transplantation: Implications for Patient Selection,” Current Opinion in Organ Transplantation 23(2): 264-270; Thieme, F. and Fronek, J. 2018. “Sharp Liver Excision under Hepatic Vascular Exclusion in Case of Liver Transplant for Large Polycystic Disease. Case Report of a New Surgical Technique,” International Journal of Surgery Case Reports 44:143-147; Liou, P. and Kato, T. 2018. “Ex Vivo Resection and Autotransplantation for Pancreatic Neoplasms,” Surgical Clinics of North America 98(1): 189-200; Lee, E., Hodgkinson, N., Fawaz, R., Vakili, K., and Kim, H, B. 2017. “Multivisceral Transplantation for Abdominal Tumors in Children: A Single Center Experience and Review of the Literature,” Pediatric Transplantation 21(5); Bharadwaj. S., Tandon, P., Gohel, T. D., Brown, J., Steiger, E., and 3 others 2017. “Current Status of Intestinal and Multivisceral Transplantation,” Gastroenterology Report (Oxford) January 26. pii: gow045; Choudhury, R. A., Reese, P. P., Goldberg, D. S., Bloom, R. D., Sawinski, D, L., and Abt. P. L. 2017. “A Paired Kidney Analysis of Multiorgan Transplantation: Implications for Allograft Survival,” Transplantation 101(2): 368-376); Argyrou, C., Moris, D., and Vernadakis, S. 2017. “Tailoring the ‘Perfect Fit’ for Renal Transplant Recipients with End-stage Polycystic Kidney Disease: Indications and Timing of Native Nephrectomy,” In Vivo (Athens, Greece) 31(3): 307-312; Caicedo, L. A., Villegas, J. I., Serrano, O., Millan, M., Sepulveda, M., and 11 others 2017. “En-bloc Transplant of the Liver, Kidney and Pancreas: Experience from a Latin American Transplant Center,” American Journal of Case Reports 18:114-118; Li, J., Guo, Q. J., Cai, J. Z., Pan, C., Shen, Z. Y., and Jiang, W. T. 2017. “Simultaneous Liver, Pancreas-duodenum and Kidney transplantation in a Patient with Hepatitis B Cirrhosis, Uremia and Insulin Dependent Diabetes Mellitus,” World Journal of Gastroenterology 23(45): 8104-8108; Choudhry, S., Dhamidharka, V. R., Castleberry, C. D., Goss, C. W., Simpson, K. E., Schechtman, K. B., and Canter, C, E. 2017. “End-stage Renal Disease after Pediatric Heart Transplantation: A 25-year National Cohort Study,” Journal of Heart and Lung Transplantation (New York, New York) pii: S1053-S2498(17) 32042-9; Grupper, A., Grupper, A., Daly, R. C., Pereira, N, L., Hathcock, M. A., and 4 others 2017. “Kidney Transplantation as a Therapeutic Option for End-stage Renal Disease Developing after Heart Transplantation,” Journal of Heart and Lung Transplantation (New York, New York) 36(3): 297-304; Samuk, I., Tekin, A., Tryphonopoulos. P., Pinto, I. G., Garcia, J., and 7 others 2016. “Abdominal Transplantation for Unresectable Tumors in Children: The Zooming Out Principle,” Pediatric Surgery International 32(4): 337-346; Modi, R. M., Patel, N., Metwally, S. N., and Mumtaz, K. 2016. “Outcomes of Liver Transplantation in Patients with Hepatorenal Syndrome,” World Journal of Hepatology 8(24): 999-1011; Habib, P. J., Patel, P. C., Hodge, D., Chimato, N., Yip, D. S., Hosenpud, J. D., and Wadei, H. M. 2016. “Pre-orthotopic Heart Transplant Estimated Glomerular Filtration Rate Predicts Post-transplant Mortality and Renal Outcomes: An Analysis of the UNOS [United Network of Organ Sharing] Database,” Journal of Heart and Lung Transplantation (New York, New York) 35(12): 1471-1479; Formica, R. N. Jr. 2016. “Simultaneous Liver Kidney Transplantation,” Current Opinion on Nephrology and Hypertension 25(6): 577-582; Yun, J. K., Choi. S. H., and Park, S. I. 2016. “Clinical Outcomes of Heart-Lung Transplantation: Review of 10 Single-center Consecutive Patients,” Korean Journal of Thoracic and Cardiovascular Surgery 49(3): 157-164; Beal, E. W., Mumtaz, K., Hayes, D. Jr., Whitson, B. A., and Black, S. M. 2016. “Combined Heart-Liver Transplantation: Indications, Outcomes and Current Experience,” Transplantation Reviews (Orlando, Florida) 30(4): 261-268; Pham. P. T., Lunsford, K. E., Bunnapradist. S., and Danovitch, G. M. 2016. “Simultaneous Liver-Kidney Transplantation or Liver Transplantation Alone for Patients in Need of Liver Transplantation with Renal Dysfunction” Current Opinion in Organ Transplantation 21(2): 194-200; Ceulemans, L. J., Strypstein, S., Neyrinck, A., Verleden, S., Ruttens. D., and 8 others 2016. “Combined Liver-thoracic Transplantation: Single-center Experience with Introduction of the ‘Liver-first’ Principle,” Transplant International 29(6): 715-726; Sharkey, L. M., Russell, N. K., Rutter, C. S., Middleton, S., Bradley, J. A., Jamieson, N. V., and Butler, A. J. 2016. “Urgent Multivisceral Transplantation for Widespread Splanchnic Ischemia,” Journal of the American College of Surgeons 222(5): 760-765: Redfield, R. R., Rickels, M. R., Naji. A., and Odorico, J. S. 2016. “Pancreas Transplantation in the Modern Era,” Gastroenterology Clinics of North America 45(1): 145-166; Rutter, C. S., Amin, I., Russell, N. K., Sharkey, L. M., Butler, A. J., and Middleton, S. J. 2016. “Adult Intestinal and Multivisceral Transplantation: Experience from a Single Center in the United Kingdom,” Transplantation Proceedings 48(2): 468-472; Sharma, P., Shu, X., Schaubel, D. E., Sung, R. S., and Magee, J, C. 2016. “Propensity Score-based Survival Benefit of Simultaneous Liver-Kidney Transplant over Liver Transplant Alone for Recipients with Pretransplant Renal Dysfunction,” Liver Transplantation 22(1): 71-79; Savla, J., Lin, K. Y., Pradhan, M., Ruebner, R, L., Rogers, R. S., and 6 others 2015, Op cit.; Sung, R. S. and Wiseman, A, C. 2015. “Simultaneous Liver-Kidney Transplant: Too Many or Just Enough?,” Advances in Chronic Kidney Disease 22(5): 399-403; Kubal. C. A., Mangus, R. S., and Tector, A. J. 2015. “Intestine and Multivisceral Transplantation: Current Status and Future Directions,” Current Gastroenterology Reports 17(1): 427; Brennan, T. V., Lunsford, K. E., Vagefi, P. A., Bostrom, A., Ma, M., and Feng, S. 2015. “Renal Outcomes of Simultaneous Liver-Kidney Transplantation Compared to Liver Transplant Alone for Candidates with Renal Dysfunction,” Clinical Transplantation 29(1): 34-43; Redfield, R. R., Scalea, J. R., and Odorico, J. S. 2015. “Simultaneous Pancreas and Kidney Transplantation: Current Trends and Future Directions,” Current Opinion in Organ Transplantation 20(1): 94-102; Kilic, A., Grimm, J. C., Whitman, G. J., Shah, A. S., Mandal, K., Conte, J. V., and Sciortino, C. M. 2015. “The Survival Benefit of Simultaneous Heart-kidney Transplantation Extends beyond Dialysis-dependent Patients,” Annals of Thoracic Surgery 99(4): 1321-1327; Puri. V. and Eason, J. 2015. “Simultaneous Liver- Kidney Transplantation,” Current Transplantation Reports 2(4): 297-302; Reich, H. J., Chan, J, L., Czer. L. S., Mirocha, J., Annamalai, A. A., and 4 others 2015. “Combined Lung-Kidney Transplantation: An Analysis of the UNOS/OPTN [United Network for Organ Sharing/Organ Procurement and Transplantation Network] Database,” American Surgeon 81(10): 1047-1052; Parajuli, S., Foley, D., Djamali. A., and Mandelbrot, D. 2015. “Renal Function and Transplantation in Liver Disease,” Transplantation 99(9): 1756-1764; Sung, R. S. and Wiseman, A, C. 2015. “Simultaneous Liver-Kidney Transplant: Too Many or Just Enough?,” Advances in Chronic Kidney Disease 22(5): 399-403; Reese, P. P., Veatch, R. M., Abt, P, L., and Amaral. S. 2014. “Revisiting Multi- organ Transplantation in the Setting of Scarcity,” American Journal of Transplantation 14(1): 21-26; Huddleston, C, B. and Richey, S, R. 2014. “Heart-Lung Transplantation,” Journal of Thoracic Disease 6(8): 1150-1158; Lachance, K., White, M., Carrier, M., Mansour, A., Racine, N., and 3 others 2014. “Long-term Evolution, Secular Trends, and Risk Factors of Renal Dysfunction following Cardiac Transplantation,” Transplant International 27(8): 824-837; Yi. S. G., Burroughs, S. G., Loebe. M., Scheinin, S. Seethamraju, H., and 9 others 2014. “Combined Lung and Liver Transplantation: Analysis of a Single-center Experience,” Liver Transplantation 20(1): 46-53; Zivkovic, S, A. 2014. “Neurologic Aspects of Multiple Organ Transplantation,” Handbook of Clinical Neurology 121:1305-1317; Jacewicz, M. and Marino, C, R. 2014. “Neurologic Complications of Pancreas and Small Bowel Transplantation,” Handbook of Clinical Neurology 121:1277-1293; Karamlou, T., Welke, K. F., McMullan, D. M., Cohen, G. A., Gelow, J., and 4 others 2014. “Combined Heart-kidney Transplant Improves Post-transplant Survival Compared with Isolated Heart Transplant in Recipients with Reduced Glomerular Filtration Rate: Analysis of 593 Combined Heart-kidney Transplants from the United Network Organ Sharing Database,” Journal of Thoracic and Cardiovascular Surgery 147(1): 456-461.e1; Mangus, R. S., Tector, A. J., Kubal, C. A., Fridell, J. A., and Vianna, R. M. 2013. “Multivisceral Transplantation: Expanding Indications and Improving Outcomes,” Journal of Gastrointestinal Surgery 17(1): 179-187; Aguilera, V., Ferrer, I., Berenguer, M., Rivera, J., Rubin, A., and 5 others 2013. “Comparison of Results of Combined Liver-Kidney Transplantation vs. Isolated Liver Transplantation,” Annals of Hepatology 12(2): 274-281; Ruzza, A., Czer, L. S., Trento, A., and Esmailian, F. 2013. “Combined Heart and Kidney Transplantation: What is the Appropriate Surgical Sequence?,” Interactive Cardiovascular and Thoracic Surgery 17(2): 416-418; Nadim, M. K., Sung, R. S., Davis, C, L., Andreoni, K. A., Biggins, S. W. and 11 others 2012. “Simultaneous Liver-Kidney Transplantation Summit: Current State and Future Directions,” American Journal of Transplantation 12(11): 2901-2908; Czer, L. S., Ruzza, A., Vespignani. R., Jordan, S., De Robertis, M. A., and 6 others 2011. “Survival and Allograft Rejection Rates after Combined Heart and Kidney Transplantation in Comparison with Heart Transplantation Alone,” Transplantation Proceedings 43(10): 3869-3876; Rana, R. K., Ghandehari. S., Falk, J. A., Simsir, S. A., Ghaly, A. S., and 6 others 2011. “Successful Combined Heart-bilateral Lung-Kidney Transplantation from a Same Donor to Treat Severe Hypertrophic Cardiomyopathy with Secondary Pulmonary Hypertension and Renal Failure: Case Report and Review of the Literature,” Transplantation Proceedings 43(7): 2820-2826).

An adult heart affected by acute decompensated systolic function (heart failure, congestive heart failure) (Steenman, M. and Lande, G. 2017. “Cardiac Aging and Heart Disease in Humans,” Biophysical Reviews 9(2): 131-137). required to pump blood to the donor heart before the recipient heart is removed, the recipient heart is prepared for transplantation for no longer than the period leading up to the removal of his heart by increasing its myocardial contractility and hemodynamic sufficiency using, as necessary, inhalation of nitric oxide, nitroglycerin oral or infused, mechanical support, such as extracorporeal membrane oxygenation, intraaortic balloon pump, Impella® (Abiomed Corporation, Danvers, Massachusetts) or similar percutaneous transluminal assist pump, or nonpercutaneous left ventricular assist device, a combination of percutaneous transluminal assist device and extracorporeal membrane oxygenation, and both L- and T-type calcium channel positive inotropes and vasodilators selected and balanced to minimize unwanted side effects. Some additional patient-to-donor line pressure can be obtained through endothelial drumming, as addressed below in the section of like title.

For the sustainment of a deceased patient sustained on life support for organ donation, the cannulae used for such prolonged extracorporeal membrane oxygenation should be replaced with conventional ductus side-entry jackets as described in U.S. Pat. No. 11,759,186, entitled Ductus Side-entry Jacket and Prosthetic Disorder Response Systems, having an internal diameter, preferably matching that of the substrate ductus and a side connector or stem angled for minimum sheer stress as described in U.S. Pat. No. 11,759,186 (see, for example, Lindholm, J, A. 2018. “Cannulation for Veno-venous Extracorporeal Membrane Oxygenation,” Journal of Thoracic Disease 10 (Supplement 5): S606-S612; Crivellari, M. and Pappalardo, F. 2018. “Femoro-jugular Cannulation in Veno-venous Extracorporeal Membrane Oxygenation PRO/CON,” Journal of Thoracic Disease 10 (Supplement 5): S613-S615; Burrell, A. J. C., Ihle, J. F., Pellegrino, V. A., Sheldrake, J., and Nixon, P. T. 2018. “Cannulation Technique: Femoro-femoral,” Journal of Thoracic Disease 10 (Supplement 5): S616-S623; Ngai, C. W., Ng. P. Y., and Sin, W, C. 2018. “Bicaval Dual Lumen Cannula in Adult Veno-venous Extracorporeal Membrane Oxygenation-Clinical Pearls for Safe Cannulation,” Journal of Thoracic Disease 10 (Supplement 5): S624-S628; Jayaraman, A, L., Cormican, D., Shah. P., and Ramakrishna, H. 2017. “Cannulation Strategies in Adult Veno-arterial and Veno-venous Extracorporeal Membrane Oxygenation: Techniques, Limitations, and Special Considerations,” Annals of Cardiac Anaesthesia 20 (Supplement): S11-S18; Reeb. J., Olland, A., Renaud, S., Lejay, A., Santelmo, N., Massard, G., and Falcoz, P, E. 2016. “Vascular Access for Extracorporeal Life Support: Tips and Tricks,” Journal of Thoracic Disease 8 (Supplement 4): S353-S363; Banfi, C., Pozzi, M., Siegenthaler, N., Brunner, M. E., Tassaux, D., and 3 others 2016. “Veno-venous Extracorporeal Membrane Oxygenation: Cannulation Techniques,” Journal of Thoracic Disease 8(12): 3762-3773).

If the medulla oblongata centers controlling respiration, or ventilation, cardiac, and vasomotor function are dead as may result from a cardiovascular accident or injury, extracorporeal membrane oxygenation or cardiopulmonary support is needed. When life support by a cardiopulmonary machine is needed, to preclude high pressure damage to the blood of the donor over a longer period as might affect the organs to be transplanted, cannulae larger in bore or diameter than the conventional are used (see, for example, von Segesser, L. K. 1999. “Cardiopulmonary Support and Extracorporeal Membrane Oxygenation for Cardiac Assist,” Annals of Thoracic Surgery 68(2): 672-677). With both sudden and metered switch techniques, machine support is always or nearly so limited to the donor, who just having died, can remain machine supported over a longer interval with little if any effect upon the recipient who is spared machine support.

The period on life support over which organs remain suitable for transplantation is pertinent only for donors not precluded for other reasons, such as age or an organ-risking condition such as metastatic cancer, Creutzfeldt-Jakob disease, atherosclerosis, chronic kidney disease, proteinurea, hepatitis, thrombophilia, polycystic kidney disease, substance abuse, hypertension, diabetes, pronounced obesity, and donor organ impairment due to systemic lupus erythematosus or sarcoidosis, for example (see, for example, Westphal, G. A., Garcia, V. D., Souza, R, L., Franke, C. A., Vieira, K. D., and 29 others 2016. “Guidelines for the Assessment and Acceptance of Potential Brain-dead Organ Donors,” Revista Brasileira de Terapia Intensiva [Brazilian Journal of Intensive Care] 28(3): 220-255; Kesimci, E., Erkilic, E., Kilinc. I., and Kilic, M. 2016. “Is an 87-year-old Grandfather Too Marginal for Being a Kidney Donor? The View of Anesthesiologists,” Journal of Clinical Medicine Research 8(9): 680-682; Lue. A., Solanas, E., Baptista, P., Lorente, S., Araiz, J. J., Garcia-Gil. A., and Serrano, M. T. 2016. “How Important is Donor Age in Liver Transplantation?,” World Journal of Gastroenterology 22(21): 4966-4976; Laging, M., Kal-van Gestel, J. A., van de Wetering J. Ijzermans, J. N., Weimar, W., and Roodnat, J. I. 2012. “The Relative Importance of Donor Age in Deceased and Living Donor Kidney Transplantation,” Transplant International 25(11): 1150-1157; Hubbard, W. J. and Dashti, N. 2011. “Aging and Transplantation-A Topic for Biomedicine or Bioethics?,” Aging and Disease 2(2): 181-185).

Moreover, “Brain death is associated with complex pathophysiological changes in cardiopulmonary function as well as fluid, electrolyte and metabolic homeostasis. [so that] . . . a precise organ-protective therapy is initiated, essentially focusing on optimal organ perfusion and oxygenation,” (Goroll, T., Gerresheim, G., Schaffartzik, W., and Schwemmer, U. 2015. “Post-mortem Organ Donation,” (in German with English abstract at Pubmed), Anaesthesist (Berlin, Germany) 64(7): 543-558).

Therapy of the prospective donor organ in the life supported donor and with the organ having been harvested with machine perfusion initiated is a science in development (see for example, Karimian, N. and Yeh, H. 2017. “Opportunities for Therapeutic Intervention During Machine Perfusion,” Current Transplantation Reports 4(2): 141-148; Sierra-Parraga, J. M., Eijken, M., Hunter, J., Moers. C., Leuvenink, H., and 5 others 2017. “Mesenchymal Stromal Cells as Anti-inflammatory and Regenerative Mediators for Donor Kidneys during Normothermic Machine Perfusion,” Stem Cells and Development 26(16): 1162-1170; Marecki, H., Bozorgzadeh, A., Porte, R. J., Leuvenink, H. G., Uygun, K., and Martins, P. N. 2017. “Liver Ex Situ Machine Perfusion Preservation: A Review of the Methodology and Results of Large Animal Studies and Clinical Trials,” Liver Transplantation 23(5): 679-695; Hamaoui, K., Gowers. S., Boutelle, M., Cook, T. H., Hanna, G., and 4 others 2016, Op cit.; Hamaoui, K., Gowers. S., Damji, S., Rogers, M., Leong, C, L., and 4 others 2016. Op cit, further references are provided below).

Critically, the direct mechanical targeting of drugs or stem cells to diseased tissue and frank lesions by means of piping alleviates the need to devise and depend upon agents with intrinsic affinity for target sites, which consideration is currently the central object of research efforts (see, for example, Fakoya, A. O. 2017. “New Delivery Systems of Stem Cells for Vascular Regeneration in Ischemia,” Online, Frontiers in Cardiovascular Medicine 4:7; Ezquer, F. E., Ezquer, M. E., Vicencio, J. M., and Calligaris, S, D. 2017. “Two Complementary Strategies to Improve Cell Engraftment in Mesenchymal Stem Cell-based Therapy: Increasing Transplanted Cell Resistance and Increasing Tissue Receptivity,” Cell Adhesion and Migration 11(1): 110-119; Naderi-Meshkin, H., Matin, M. M., Heirani-Tabasi. A., Mirahmadi, M., Irfan-Maqsood, M., and 6 others 2016. “Injectable Hydrogel Delivery Plus Preconditioning of Mesenchymal Stem Cells: Exploitation of SDF-1/CXCR4 [stromal cell-derived factor 1/N-terminal cysteine-amino acid-N-terminal cysteine chemokine receptor type 4] Axis Toward Enhancing the Efficacy of Stem Cells' Homing,” Cell Biology International 40(7): 730-741; Bidkhori, H. R., Ahmadiankia, N., Matin, M. M., Heirani-Tabasi, A., Farshchian, M., and 4 others 2016. “Chemically Primed Bone-marrow Derived Mesenchymal Stem Cells Show Enhanced Expression of Chemokine Receptors Contributed to Their Migration Capability,” Iran Journal of Basic Medical Sciences19(1): 14-19; Kallmeyer, K. and Pepper, M. S. 2015. “Homing Properties of Mesenchymal Stromal Cells,” Expert Opinion on Biological Therapy 15(4): 477-479; De Becker, A. and Riet, I. V. 2016. “Homing and Migration of Mesenchymal Stromal Cells: How to Improve the Efficacy of Cell Therapy?,” World Journal of Stem Cells 8(3): 73-87; Cornelissen, A. S., Maijenburg, M. W., Nolte. M. A., and Voermans, C. 2015. “Organ-specific Migration of Mesenchymal Stromal Cells: Who, When, Where and Why?,” Immunology Letters 168(2): 159-169; Vicencio, J. M., Yellon, D. M., Sivaraman, V., Das, D., Boi-Doku, C., and 8 others 2015. “Plasma Exosomes Protect the Myocardium from Ischemia-reperfusion Injury,” Journal of the American College of Cardiology 65(15): 1525-1536; Chen, L., Wang. Y., Pan, Y., Zhang, L., Shen, C., and 5 others 2013. “Cardiac Progenitor-derived Exosomes Protect Ischemic Myocardium from Acute Ischemia/Reperfusion Injury,” Biochemical and Biophysical Research Communications 431(3): 566-571).

Essentially, machine support can continue with periodic replacement of the blood up to the point where donor organs become adversely affected, newer apparatus extending this period (see, for example. Rubio, O., Amau, A., Cano, S., Subira. C., Balerdi, B., and 39 others 2018. “Limitation of Life Support Techniques at Admission to the Intensive Care Unit: A Multicenter Prospective Cohort Study,” Journal of Intensive Care 6:24; Bronchard, R., Durand, L., Legeai. C., Cohen, J., Guerrini, P., and Bastien, O. 2017, Op cit., above in section 2a, entitled Background. General: Eichhom, S., Spindler, J., Polski, M., Mendoza, A., Schreiber, U., and 5 others 2017. “Development and Validation of an Improved Mechanical Thorax for Simulating Cardiopulmonary Resuscitation with Adjustable Chest Stiffness and Simulated Blood Flow,” Medical Engineering and Physics 43:64-70; Butt. W. and MacLaren, G. 2016. “Extracorporeal Membrane Oxygenation 2016: An Update,” F1000Research 5. pii: F1000 Faculty Rev-750; Butt, W. and Maclaren, G. 2016. “Concepts from Paediatric Extracorporeal Membrane Oxygenation for Adult Intensivists,” Annals of Intensive Care 6(1): 20; Singh, G., Tsukashita, M., Biscotti, M., Costa, J., Lambert, D., Bacchetta, M., and Takayama, H. 2016. “Heart Procurement from a Donor on Venovenous ECMO [Extracorporeal Membrane Oxygenation] Support,” American Society for Artificial Internal Organs Journal 62(3): e24-e26; Liu, Y., Sanchez, P. G., Nam, S. J. and Cho, Y. J. 2016. “The Successful Treatment of Refractory Respiratory Failure Due to Miliary Tuberculosis: Survival after Prolonged Extracorporeal Membrane Oxygenation Support,” Clinical Respiratory Journal 10(3): 393-399; Lee. H., Cho, Y. H., Sung, K., Yang, J. H., Chung, C. R., Jeon, K., and Suh, G. Y. 2015. “The Use of Extracorporeal Circulation in Suspected Brain dead Organ Donors with Cardiopulmonary Collapse,” Journal of Korean Medical Science 30(12): 1911-1914; Arroyo, D., Gasche, Y., Banfi. C., Stiasny, B., Bendjelid, K., and Giraud, R. 2015. “Successful Heart Transplantation after Prolonged Cardiac Arrest and Extracorporeal Life Support in Organ Donor—A Case Report,” Journal of Cardiothoracic Surgery 10:186; Hernandez-Tejedor. A., Martin Delgado, M. C., Cabre Pericas, L., and Algora Weber A., with 95 collaborators 2015. “Limitation of Life-sustaining Treatment in Patients with Prolonged Admission to the ICU. Current Situation in Spain as Seen from the EPIPUSE [Evolución y pronostico de los pacientes con ingreso prolongado en UCI que sufren un empeoramiento/Evolution and prognosis of long intensive care unit stay patients suffering a deterioration] Study,” Medicina Intensiva (Madrid, Spain) 39(7): 395-404; Balsorano, P., Ciapetti, M., Cianchi, G., Bonizzoli, M., Migliaccio, M, L., and 3 others 2015. “Extracorporeal Life Support and Multiorgan Donation in a Severe Polytrauma Patient: A Case Report,” International Journal of Surgery Case Reports 9:109-111; Wei, X., Watkins, A. C., Niu, S., Wu, Z. J., and Griffith, B. P. 2015. “Effects of Cardiopulmonary Support with a Novel Pediatric Pump-lung in a 30-Day Ovine Animal Model,” Artificial Organs 39(12): 989-997; Tramm, R., Ilic. D., Davies, A. R., Pellegrino, V. A., Romero, L., and Hodgson, C. 2015. “Extracorporeal Membrane Oxygenation for Critically Ill Adults,” Cochrane Database of Systematic Reviews 1: CD010381; Kon, Z. N., Dahi, S., Evans, C. F., Bymes, K. A., Bittle, G. J., and 7 others 2015. “Long-term Venovenous Extracorporeal Membrane Oxygenation Support for Acute Respiratory Distress Syndrome,” Annals of Thoracic Surgery 100(6): 2059-2063; Carter. T., Bodzin, A. S., Hirose, H., West, S., Hasz, R., Maley, W. R., and Cavarocchi, N, C. 2014. “Outcome of Organs Procured from Donors on Extracorporeal Membrane Oxygenation Support: An Analysis of Kidney and Liver Allograft Data,” Clinical Transplantation 28(7): 816-820; Yang, H. Y., Lin, C. Y., Tsai, Y. T., Lee, C. Y., and Tsai, C. S. 2012. “Experience of Heart Transplantation from Hemodynamically Unstable Brain-dead Donors with Extracorporeal Support,” Clinical Transplantation 26(5): 792-796; Hannan, R, L., Ojito, J. W., Ybarra, M. A., O'Brien, M. C., Rossi, A. F., and Burke, R. P. 2006. “Rapid Cardiopulmonary Support in Children with Heart Disease: A Nine-year Experience,” Annals of Thoracic Surgery 82(5): 1637-1641; von Segesser, L. K. 1999, Op cit.).

The recipient is also isolated from serious mechanical and medical mishaps during machine support of the donor (see, for example, Lee, S. H., Jung, J. S., Chung, J. H., Lee, K. H., Kim, H. J., Son. H. S., and Sun, K. 2015. “Right Heart Failure during Veno-venous Extracorporeal Membrane Oxygenation for HINI [hemagglutinin 1 neuraminidase 1] Induced Acute Respiratory Distress Syndrome: Case Report and Literature Review,” Korean Journal of Thoracic and Cardiovascular Surgery 48(4): 289-293). With respect to both heart and other solid organ transplantation moreover, there is no reperfusion, thus instantly eliminating a significant source of complications (see, for example, White, C. W., Ambrose, E., Müller, A., Li, Y., Le, H. and 8 others 2016, Op cit.; Farine, E., Niederberger, P., Wyss, R. K., Mendez-Carmona, N., Gahl. B., and 4 others 2016 Op cit.).

The preferred order in which to harvest and the best means for maintaining the different organs is less clear for kidneys (see, for example, Weissenbacher, A. and Hunter, J. 2017. “Normothermic Machine Perfusion of the Kidney,” Current Opinion in Organ Transplantation 22(6): 571-576; Ravaioli, M., De Pace, V., Comai, G., Busutti, M., Del Gaudio, M., and 6 others 2017. “Successful Dual Kidney Transplantation after Hypothermic Oxygenated Perfusion of Discarded Human Kidneys,” American Journal of Case Reports 18:1009-1013; Blum, M. F., Liu, Q., Soliman, B., Dreher. P., Okamoto. T., and 4 others 2017. “Comparison of Normothermic and Hypothermic Perfusion in Porcine Kidneys Donated after Cardiac Death,” Journal of Surgical Research 216:35-45; Kaths. J. M., Echeverri, J., Chun, Y. M., Cen, J. Y., Goldaracena, N., and 10 others 2017. “Continuous Normothermic Ex Vivo Kidney Perfusion Improves Graft Function in Donation after Circulatory Death Pig Kidney Transplantation,” Transplantation 101(4): 754-763; Paloyo, S., Sageshima, J., Gaynor, J. J., Chen, L., Ciancio, G., and Burke, G. W. 2016. “Negative Impact of Prolonged Cold Storage Time before Machine Perfusion Preservation in Donation after Circulatory Death Kidney Transplantation” Transplant International 29(10): 1117-1125; Yong. C., Hosgood, S. A., and Nicholson, M. L. 2016. “Ex-vivo Normothermic Perfusion in Renal Transplantation: Past, Present, and Future,” Current Opinion in Organ Transplantation 21(3): 301-307; Jiao. B., Liu, S., Liu, H., Cheng. D., Cheng. Y., and Liu, Y. 2013. “Hypothermic Machine Perfusion Reduces Delayed Graft Function and Improves One-year Graft Survival of Kidneys from Expanded Criteria Donors: A Meta-analysis,” Online, PLoS [Public Library of Science] One 8(12): e81826).

Sustainment in the intact donor of cardiopulmonary function over a longer period with zero ischemic time under normothermic conditions is preferable for the heart and lungs—where pulmonary hypertension due to inflammation in the arteries of the lungs in the recipient would induce graft failure were the heart alone replaced, the lungs are preferably transplanted together with the heart—and the liver, over either chilled machine perfusion, and more still than static storage in a cardioplegic solution (see, for example, Ceresa, C, D, L., Nasralla, D., and Jassem, W. 2018. “Normothermic Machine Preservation of the Liver: State of the Art,” Current Transplantation Reports 5(1): 104-110; Schlegel, A., Muller, X., and Dutkowski, P. 2018. “Hypothermic Machine Preservation of the Liver: State of the Art,” Current Transplantation Reports 5(1): 93-102; Nostedt, J. J., Skubleny, D. T., Shapiro, A. M. J., Campbell, S., Freed, D. H., and Bigam, D. L. 2018. “Normothermic Ex Vivo Machine Perfusion for Liver Grafts Recovered from Donors after Circulatory Death: A Systematic Review and Meta-Analysis,” Hepatic, Pancreatic, and Biliary Surgery 2018:6867986; Delsuc. C., Faure, A., Berthiller, J., Dorez, D., Matillon, X., and 12 others 2018. “Uncontrolled Donation after Circulatory Death: Comparison of Two Kidney Preservation Protocols on Graft Outcomes,” Online, BioMed Central Nephrology 19(1): 3; Boteon, Y, L., Afford, S. C., and Mergental, H. 2018. “Pushing the Limits: Machine Preservation of the Liver as a Tool to Recondition High-risk Grafts,” Current Transplantation Reports 5(2): 113-120; Jayant, K., Reccia, I., Virdis, F., and Shapiro, A. M. J. 2018, Op cit.; Watson, C. J. E., Kosmoliaptsis. V., Randle, L. V., Gimson, A. E., Brais, R., and 4 others 2017. “Normothermic Perfusion in the Assessment and Preservation of Declined Livers Before Transplantation: Hyperoxia and Vasoplegia-Important Lessons from the First 12 Cases,” Transplantation 101(5): 1084-1098; Slama, A., Schillab, L., Barta, M., Benedek, A., Mitterbaue, A., and 9 others 2017. “Standard Donor Lung Procurement with Normothermic Ex Vivo Lung Perfusion: A Prospective Randomized Clinical Trial,” Journal of Heart and Lung Transplantation (New York, New York) 36(7): 744-753; Gill, J., Rose, C., Lesage, J., Joffres, Y., Gill, J., and O'Connor, K. 2017. “Use and Outcomes of Kidneys from Donation after Circulatory Death Donors in the United States,” Journal of the American Society of Nephrology 28(12): 3647-3657; Hameed, A. M., Hawthorne, W. J., and Pleass, H, C. 2017. “Advances in Organ Preservation for Transplantation,” Australian and New Zealand Journal of Surgery 87(12): 976-980; Zhong, Z., Lan, J., Ye. S., Liu, Z., Fan, L., and 6 others 2017. “Outcome Improvement for Hypothermic Machine Perfusion Versus Cold Storage for Kidneys from Cardiac Death Donors,” Artificial Organs 41(7): 647-653; Wang. W., Xie. D., Hu. X., Yin, H., Liu, H., and Zhang, X. 2017. “Effect of Hypothermic Machine Perfusion on the Preservation of Kidneys Donated after Cardiac Death: A Single-center, Randomized, Controlled Trial,” Artificial Organs 41(8): 753-758; Scalea, J. R., Redfield, R. R., Arpali, E., Leverson, G. E., Bennett, R. J., and 6 others 2017. “Does DCD [donation after circulatory death] Donor Time-to-death Affect Recipient Outcomes? Implications of Time-to-death at a High-volume Center in the United States,” American Journal of Transplantation 17(1): 191-200; Reeb, J. and Cypel, M. 2016, Op cit. above under section 2a(3); Hameed, A. M., Pleass, H. C., Wong, G., and Hawthorne, W. J. 2016. “Maximizing Kidneys for Transplantation Using Machine Perfusion: From the Past to the Future: A Comprehensive Systematic Review and Meta-analysis,” Medicine (Baltimore, Maryland) 95(40): e5083; Demiselle, J., Augusto, J. F., Videcoq, M., Legeard, E., Dube, L., and 6 others 2016. “Transplantation of Kidneys from Uncontrolled Donation after Circulatory Determination of Death: Comparison with Brain Death Donors with or without Extended Criteria and Impact of Normothermic Regional Perfusion,” Transplant International 29(4): 432-442; Paloyo, S., Sageshima, J., Gaynor, J. J., Chen, L., Ciancio, G., and Burke, G. W. 2016. “Negative Impact of Prolonged Cold Storage Time before Machine Perfusion Preservation in Donation after Circulatory Death Kidney Transplantation,” Transplant International 29(10): 1117-1125; Summers, D. M., Watson, C. J., Pettigrew, G. J., Johnson, R. J., Collett, D., Neuberger, J. M., and Bradley, J, A. 2015. “Kidney Donation after Circulatory Death (DCD): State of the Art,” Kidney International 88(2): 241-249; Hosgood, S. A., van Heum, E., and Nicholson, M. L. 2015. “Normothermic Machine Perfusion of the Kidney: Better Conditioning and Repair?,” Transplant International 28(6): 657-664; Nagaraja, P., Roberts, G. W., Stephens, M., Horvath, S., Kaposztas, Z., Chavez, R., and Asderakis, A. 2015. “Impact of Expanded Criteria Variables on Outcomes of Kidney Transplantation from Donors after Cardiac Death,” Transplantation 99(1): 226-231; Nicholson, M. L. and Hosgood, S, A. 2013. “Renal Transplantation after Ex Vivo Normothermic Perfusion: The First Clinical Study,” American Journal of Transplantation 13(5): 1246-1252; Bathini, V., McGregor, T., McAlister, V. C., Luke, P. P., and Sener, A. 2013. “Renal Perfusion Pump vs Cold Storage for Donation after Cardiac Death Kidneys: A Systematic Review,” Journal of Urology (Philadelphia, Pennsylvania) 189(6): 2214-2220; Deng, R., Gu, G., Wang, D., Tai, Q., Wu, L., and 4 others 2013. “Machine Perfusion Versus Cold Storage of Kidneys Derived from Donation after Cardiac Death: A Meta-analysis,” Online, PLoS [Public Library of Science] One 8(3): e56368).

For this reason, the heart is best kept in the donor on life support as long as possible, sustainment in the donor of the heart with the aid of inotropic drugs addressed below. That other than residua blood in the transplant organ the blood of the donor is not passed to the recipient but rather the reverse and that the patient is not subjected to extended machine support evades numerous potential complications. More generally, operative time is a significant factor for adverse sequelae, and sudden compound vascular bypass, or sudden switched-organ transplantation takes less time than does conventional transplantation.

To the trauma inflicted on both donor and recipient hearts using a conventional technique must be added the fact of extended time. Furthermore, long duration machine support during conventional heart transplantation can have grave hematological and neurological consequences; however, these are not transferred but rather kept separate from as not to affect the donor. At the same time, preservation intact of the donor is significant, in that other organs for transplantation are better protected.

Whereas the ability to introduce anticlotting and antibiofilm agents into synthetic tubing through the jacket drugline can extend complication-free service to the end of life, the thrombolytic sol-gel coatings on the internal surface of artificial vessels (Chapurina, Y., Vinogradov, V. V., Vinogradov, A. V., Sobolev, V. E., Dudanov, I. P., and Vinogradov, V. V. 2015. “Synthesis of Thrombolytic Sol-Gel Coatings: Toward Drug-Entrapped Vascular Grafts,” Journal of Medicinal Chemistry 58(15): 6313-6317) have a finite life so that the drug may be depleted well before the life of the tubing itself. Moreover, the internal coatings are not replenishable without introducing a thrombolytic such as tissue plasminogen activator, urokinase, or streptokinase which will be dispersed throughout the circulatory system and not targeted to the artificial tubing.

2a(4). Central Lines

Long-term if not permanent connection by a side-entry jacket or nonjacketing side-entry connector of the distal end of a central line to allow monitoring or drug delivery over a long period securely fastens the line to the substrate ductus, eliminating concerns about a puncture or perforation at the distal end. The line can therefore be positioned without concern for a dissection or perforation as can occur with an active pediatric patient. A central line to diagnose and/or treat a chronic disease must remain dependable completely and permanently to allow bathing, swimming, and participation in non-impact sports with little concern.

Placement of a conventional central or peripherally inserted central line is less invasive but subject to more complications in placement and ensuing use than a side-entry jacket or side-entry diversion jacket. With side-entry and diversion side-entry jackets, access to the target vessel is by direct viewing through a small incision, substantially reducing if not eliminating misidentification or failure in correctly approaching the vessel intended to cause injury elsewhere. Placement is less injurious to the target vessel, reducing if not eliminating complications such as thrombosis, and following placement, the jacket, which fully complies with intrinsic movement of the pulse, remains fixed in position, so that the patient can move freely.

Catheter fracture and migration, a rare complication with implanted central lines and pleural catheters, is avoided through the use of catheters made of materials both strong and highly pliant. Drugs for automatic release by an implanted disorder response system as described in U.S. Pat. No. 11,759,186, entitled Ductus Side-entry Jackets and Prosthetic Disorder Response Systems must be formulated as fluids. Freely flowing fluids to be released while the patient is upright can be released by gravity, higher viscosity fluids driven out of the drug reservoir and forward by the reservoir outlet pump.

Secondary lines, (druglines, accessory channels) allow flow in the same or the opposite direction as that through the mainline and isolate medication delivered from the mainline. Access to the central line can be through a subdermally implanted port such as shown in FIGS. 26A and 26C, or the cutaneous port shown in FIG. 45 of U.S. Pat. No. 11,759,186, entitled Ductus Side-entry Jackets and Prosthetic Disorder Response Systems, which can lead directly to the substrate vessel or to replenish a drug storage reservoir implanted subdermally in the pectoral region for automatically controlled release by a fully implanted response system.

For excurrent as opposed to incurrent lines, an above-skin, or cutaneous, opening in the port allows a bodily fluid to be drained off into a collection bag. A single port can incorporate both above-skin and subdermal openings. The port requires at least two openings, one for the main drugline, the other for supportive substances such as an anticoagulant. Connection to the substrate vessel with a basic side-entry jacket or vascular servovalve rather than a hollow needle or hypotube allows delivery into the line, jacket, and vessel of antimicrobials, anticoagulants, and crystal deposition counteractants through the accessory channel or channels of each such connector.

Central lines connected by side-entry jackets or vascular valves are minimally double or double lumen-one lumen or line to serve as the mainline to pass drugs, blood, or parenteral nutrition. The other lumen or lumina to serve as sidelines, or druglines, which feed into the accessory channel or channels—to supply water, antimicrobials, anti-inflammatoires, anticoagulants, and so on through the jacket and into the vessel to which the side-entry jacket is connected. Prevention of parenteral nutrition line occlusion is by occasional flushing of the line with water. In an application administered by an automatic fully implanted disorder response system, the preservation of line patency thus can be made automatic.

As will be addressed, unlike a conventional central line or central venous catheter, peripherally inserted central catheters, non-tunneled, tunneled, and totally implanted venous access catheters, the internal end of the catheter connected to the vessel by a side-entry or side-entry diversion jacket is not suspended as a foreign body within the native lumen where it presents a cross-sectional profile that continuously changes longitudinally, causing turbulent flow, promoting thrombosis, but rather holds the distal tip of the catheter in flush alignment with the endothelium, thus little if at all disrupting streamline or laminar flow passing the tiny hole in the side of the vessel. Using conventional means, thrombosis is due to turbulence, immune response to the injury of puncturing and entering into, and the presence of a foreign body within, the lumen.

This applies regardless of the reason for placing the central or peripheral such as femoral line, whether to infuse chemotherapy, provide parenteral nutrition, or to connect an extracorporeal therapy machine, such as a dialyzer. The advantages in using a central or peripheral line that connects a special port at the body surface with entry into the native lumen through a ductus or diversion side-entry jacket generally relate to shortcomings historically associated with the use of conventional lines rather than the process supported.

For example, the use of a central line as delineated herein for total parenteral nutrition avoids much if not all thrombosis and the greater risk of infection with a conventional line but not the numerous problems intrinsic in long-term total parenteral nutrition, such as constant hunger, fatty liver, intrahepatic cholestasis, cholecystitis, cholelithiasis, and intestinal atrophy. A side-entry jacket or side-entry diversion jacket can be used to establish a long-term or permanent central line to deliver an infusate for dispersion throughout the body or to directly target a particular level along a vessel of the gut, for example, and a side-entry diversion jacket includes a means for adjusting the rate of flow through the line.

The line is entered for injection through a small subdermal port at the body surface, or if a higher flow rate is needed, through an above-skin opening in a port of which only the infusion opening is above skin. Systemic use is for any purpose served by conventional central lines, to include hemodialysis, apheresis, parenteral nutrition, and chemotherapy, for example. Connection to the target vessel is through the jacket or diversion jacket. Whether side-entry diversion jackets should replace central lines with body surface port access depends upon the length of time the line will remain in place and the potential for complications in the specific patient.

Suspension of the distal end of the line within the vessel so that it is left hanging unconnected in the lumen is responsible for numerous serious complications to which side-entry jackets and side-entry diversion jackets are not, to include not only thrombosis, but stenosis, tissue ingrowth, vascular erosion, fracture, fragmentation, and migration. When a central line will be needed indefinitely and access and eventual removal through a small incision would allow securing the otherwise free end, the potential complications in using either approach should be considered.

Otherwise, central lines can cause many problems (see, for example, Liu, W., Wei, W., Wang. Y., Liu, B., Pang. Y., and Sun, W. 2023. “Spontaneous Migration of a Peripherally Inserted Central Catheter into the Azygos Vein during Postoperative Gastrointestinal Dysmotility: A Case Report,” Online, Medicine (Baltimore, Maryland) 102(22): e33921; Wang. Y. and Zhao, L. 2022. “Diagnosis and Management of Secondary Malposition of PICC into the Azygos Vein.,” Journal of Vascular Access (Thousand Oaks, California) 23(3): 450-454; Talari, G., Talari, P., Parasramka, S., and Mirrakhimov, A, E. 2018. “Recurrent Migration of Peripherally Inserted Central Catheter into the Azygos Vein” Online, BMJ [British Medical Journal] Case Reports (London, England) 2018: bcr2017221184; Grau, D., Clarivet, B., Lotthe, A., Bommart, S., and Parer, S. 2017. “Complications with Peripherally Inserted Central Catheters (PICCs) Used in Hospitalized Patients and Outpatients: A Prospective Cohort Study,” Antimicrobial Resistance and Infection Control 6:18; Bertoglio, S., Faccini, B., Lalli, L., Cafiero, F., and Bruzzi, P. 2016. “Peripherally Inserted Central Catheters (PICCs) in Cancer Patients Under Chemotherapy: A Prospective Study on the Incidence of Complications and Overall Failures,” Journal of Surgical Oncology 113(6): 708-714; Blackwood, B. P., Farrow, K. N., Kim, S., and Hunter, C. J. 2016. “Peripherally Inserted Central Catheters Complicated by Vascular Erosion in Neonates,” JPEN Journal of Parenteral and Enteral Nutrition (Hoboken, New Jersey) 40(6): 890-895; Kovacich, A., Tamma, P. D., Advani. S., Popoola, V. O., Colantuoni, E., Gosey, L., and Milstone, A. M. 2016. “Peripherally Inserted Central Venous Catheter Complications in Children Receiving Outpatient Parenteral Antibiotic Therapy (OPAT),” Infection Control and Hospital Epidemiology 37(4): 420-424; Leung, A., Heal. C., Banks. J., Abraham, B., Capati. G., and Pretorius, C. 2016. “The Incidence of Peripheral Catheter-related Thrombosis in Surgical Patients,” Thrombosis 2016: 6043427; Nolan, M. E., Yadav, H., Cawcutt. K. A., and Cartin-Ceba, R. 2016. “Complication Rates Among Peripherally Inserted Central Venous Catheters and Centrally Inserted Central Catheters in the Medical Intensive Care Unit,” Journal of Critical Care 31(1): 238-242; Peng, J., Zhang, X. M., Yang, L., Xu, H., Miao, N. D., Ren, Y. J. Liu, K., and 4 others 2016. “A Novel Two-Step Technique for Retrieving Fractured Peripherally Inserted Central Catheter Segments Migrating into the Heart of the Pulmonary Artery,” Online, Biomed Research International 2016:7814529; Terry, C. M., He. Y., and Cheung, A. K. 2016. “Rivaroxaban Improves Patency and Decreases Inflammation in a Mouse Model of Catheter Thrombosis,” Thrombosis Research 144:106-112; Beccaria, P., Silvetti, S., Mucci, M., Battini, I., Brambilla. P., and Zangrillo, A. 2015. “Contributing Factors for a Late Spontaneous Peripherally Inserted Central Catheter Migration: A Case Report and Review of [the] Literature,” Journal of Vascular Access (Thousand Oaks, California) 16(3): 178-182; Kombau, C., Lee, K. C., Hughes, G. D., and Firstenberg, M. S. 2015. “Central Line Complications,” International Journal of Critical Illness and Injury Science 5(3): 170-178; Mitsopoulos, G., Hanna, R. F., Brejt. S. Z., Goldstein, G. E., Sheynzon, V., Weintraub, J, L., and Gray, W, A. 2015. “Retrieval of a Dislodged Catheter Using Combined Fluoroscopy and Intracardiac Echocardiography,” Case Reports in Radiology 2015:610362; Wang, Q., Xiong, B., Zheng, C., Feng, G., Liang. M., and Liang, H. 2015. “Percutaneous Retrieval of PICC [peripherally inserted central catheter] Fractures via the Femoral Vein in Six Cancer Patients,” Journal of Vascular Access (Thousand Oaks, California) 16(1): 47-51; Zhang, H., Jiang. B., Zhou, H., Qin, Y., and Wang, G. 2015. “Spontaneous Migration of a Peripherally Inserted Central Catheter into the Azygos Vein in Esophageal Cancer Patients during Surgery,” Journal of Vascular Access (Thousand Oaks, California) 16(6): 537-539; Chopra. V., Ratz, D., Kuhn, L., Lopus. T., Lee. A., and Krein, S. 2014. “Peripherally Inserted Central Catheter-related Deep Vein Thrombosis: Contemporary Patterns and Predictors,” Journal of Thrombosis and Haemostasis 12(6): 847-854; Zochios, V., Umar. I., Simpson, N., and Jones, N. 2014. “Peripherally Inserted Central Catheter (PICC)-related Thrombosis in Critically Ill Patients,” Journal of Vascular Access (Thousand Oaks, California) 15(5): 329-337; Cahill, A. M., Jumani, K., Advani. S., Reich, N. G., Gosey, L., and Milstone, A. M. 2013. “Risk Factors for Peripherally Inserted Central Venous Catheter Complications in Children,” JAMA [Journal of the American Medical Association] Pediatrics 167(5): 429-435; Wrightson, D, D. 2013. “Peripherally Inserted Central Catheter Complications in Neonates with Upper Versus Lower Extremity Insertion Sites,” Advances in Neonatal Care 13(3): 198-204; Alkindi, S., Matwani, S., Al-Maawali, A., Al-Maskari, B., and Pathare, A. 2012. “Complications of Port-A-Cathe in Patients with Sickle Cell Disease,” Journal of Infection and Public Health 5(1): 57-62; Cahill, A. M., Ballah, D., Hernandez, P., and Fontalvo, L. 2012. “Percutaneous Retrieval of Intravascular Venous Foreign Bodies in Children,” Pediatric Radiology 42(1): 24-31; Lee, A. Y., and Kamphuisen, P. W. 2012. “Epidemiology and Prevention of Catheter-related Thrombosis in Patients with Cancer,” Journal of Thrombosis and Haemostasis 10(8): 1491-1499; Turcotte, S., Dube, S., and Beauchamp, G. 2006. “Peripherally Inserted Central Venous Catheters are not Superior to Central Venous Catheters in the Acute Care of Surgical Patients on the Ward” World Journal of Surgery 30(8): 1605-1619; Fazeny-Dorner, B., Wenzel. C., Berzlanovich, A., Sunder-Plassmann, G., Greinix, H., Marosi, C., and Muhm, M. 2003. “Central Venous Catheter Pinch-off and Fracture: Recognition, Prevention and Management,” Bone Marrow Transplantation 31(10): 927-930).

In placing central lines, the wire used to guide the catheter or line into the vessel is as much a source of complications as is the line itself (see, for example, Padiyath, A., Fontenot, E. E., and Abraham, B. P. 2017. “Removal of a Retained Intracardiac Radiolucent Guidewire Fragment Using an Atrieve™ Vascular Snare Using Combined Fluoroscopy and Transesophageal Echocardiography Guidance in an Infant,” Annals of Pediatric Cardiology 10(1): 65-68; Peng, J., Zhang, X. M., Yang, L., Xu. H., Miao. N. D., Ren, Y. J. Liu, K., and 4 others 2016. Op cit.; Woodhouse, J, B. and Uberoi, R. 2013. “Techniques for Intravascular Foreign Body Retrieval,” Cardiovascular and Interventional Radiology 6(4): 888-897; Cahill, A. M., Bandllah, D., Hernandez, P., Fontalvo L. 2012, Op cit. this section).

When the central line is or is also used to draw blood samples, the delivery of an anticoagulant through the accessory channel prevents clot, which the placement of the line itself promotes, from clogging the line. Double jacketing thus also means that the segment of the ductus beginning at the level of the upper jacket can be infused at a drug concentration intended to treat the affected segment of the ductus, which if harmful to other tissue so that it should not be allowed to continue through the circulatory system, can be neutralized by a reversal agent delivered through the distal jacket. By the same token, if the drug or drugs are needed systemically but at a lower dose, the degree to which these are neutralized can be reduced as necessary.

With an anticoagulant delivered through a central line, or central venous catheter, from the body surface port and into a vessel through a side-entry or diversion jacket, provided a reversal agent is available, a second jacket positioned at the distal margin of the central line to be kept free of thrombus can deliver a reversal agent, substantially constraining the anticoagulant to the central line-vessel junction, thus minimizing continued flow of the anticoagulant through the circulatory system—vitamin K1 (phylloquinone) or vitamin K2 (menaquinone) to reverse warfarin (Coumadin), or protamine sulfate to reverse heparin.

Otherwise, a systemically circulated higher dose of an anticoagulant such as heparin has the potential to induce serious side effects, among which are heparin-induced thrombocytopenia, hemorrhage, and an elevated blood potassium level, or hyperkalemia, resulting in cardiac arrhythmia, weakness, and malaise. Bypass about an impaired segment of a major vessel such as the abdominal aorta or inferior vena cava allows that segment to be spared further exposure to injurious or infectious substances or drugs in the blood given to treat other tissue where apheresis or chelation therapy is neither appropriate nor warranted. Blood-borne pathogens, bacteremia, viremia, fungemia, and parasitemia can all further injure an impaired vessel (see, for example, Kozarov, E. 2012. “Bacterial Invasion of Vascular Cell Types: Vascular Infectology and Atherogenesis” Future Cardiology 8(1): 123-138).

Side-entry and side-entry valve-jackets can be used to create long-term or permanent externally controllable central lines capable of any longer than temporary function served by conventional lines and can do so with greater versatility and safety. While a central line connected by a side-entry jacket is not inserted by direct puncture but rather through a ‘keyhole’ incision, the avoidance of leakage into neighboring tissue on placement and thereafter can represent a significant benefit. In transplantation, provided donor and recipient are positioned side by side, placement of side-entry diversion jackets on the incurrent and excurrent vessels of both donor and recipient organs allow the immediate switching of perfusion through the donor organ from the bloodstream of the donor to that of the parecipient.

Placement of a central line aside, to the extent that a patient, as when suffering from cancer, is more prone to clotting, an anticoagulant must be systemically circulated. However, to the extent that the central line aggravates the tendency to clot as a consequence of the trauma caused to the vessel in placing the line (Polderman, K, H. and Girbes, A. J. 2002. “Central Venous Catheter Use. Part 1: Mechanical Complications,” Intensive Care Medicine 28(1): 1-17) and due to the turbulent flow of the blood caused when passing a foreign object suspended in the lumen, this tendency can be reduced if not eliminated by constraining the delivery drugs in more concentrated dose directly to the line insertion site and the affected segment of the ductus moving away from the incision, puncture, and/or lesion caused by disease, treatment of the injury of insertion with an anti-inflammatory and cytokine cascade proliferative-counteractive drugs and of clotting with an anticoagulant.

Currently, conventional methods for gaining immediate access for connection to a hemodialysis or hemapheresis machine are insertion of a central line, or central venous catheter connected to the jugular or subclavian vein or a (noncentral) line connected to a femoral vein. Of the three methods available for gaining access, this approach is also that most invasive and susceptible to complications. The other two methods are to surgically create an arteriovenous fistula or an arteriovenous graft. Of the two, the first cannot be used for about five weeks and the graft for about two weeks.

The patient must therefore undergo not one but two surgical procedures, and once a compatible transplant kidney is found, a third procedure. Until the fistula or graft is created, the patient will have a tube extending out of his body and require another venipuncture three to five if not seven times a week. A peripherally inserted central catheter (PICC line) or totally implantable venous access device with subdermally implanted mediport or portacath entry is able to pass hypodermic needles through the skin and the device front membrane but not a catheter, so that the port is a barrier to the passage therethrough of the catheter, which is totally implanted and not retrievable through the port. This makes it suitable for chemotherapy, for example, but not passing through lines for hemodialysis or hemapheresis, for example.

Moreover, with any central venous or femoral catheter, the distal end of the line, as documented below, is neither secured nor stable, disallowing participation in energetic physical movement of exercise. A central line does, however, eliminate the need for repeated venipuncture and can preserve connection to vessels large in cross-sectional area. Side-entry diversion jackets with entry port implanted subdermally combine a means for immediate connection to the extracorporeal machine and used with the use of a high flux rate dialyzer, for example, considerably reduces the time needed for each treatment. Moreover, unlike a conventional central line, the initial access need never be replaced. If a transplant kidney is found, the surface port and jacket are used to target drugs directly to and in support of the transplant.

Conventionally, initial placement of a central line, whether to administer chemotherapy, connect the patient to a hemodialysis/hemapheresis machine, or provide parenteral nutrition, for example, requires 1. Placing the patient on a tilting table or other angleable surface, 2. Adjusting the table to a partial Trendelenburg position, 3. Sterilizing the site, usually in the cervical or femoral region, with a topical antiseptic, 4. Injecting an anesthetic at the prospective insertion site, 5. Locating the vein for line insertion with the aid of an ultrasound scanner, 6. Inserting a guide wire needle to enter the vein, 7. Passing a guidewire through the guidewire needle and into the vein, 8. Passing the line, or catheter, over the guidewire through the integument and into the lumen of the vein, 9. Withdrawing the guidewire, 10. Fixing the catheter in position with suture or tape, and 11. Due to the proximity of neighboring structures, jugular or subclavian insertion is examined radiologically to confirm proper placement and the absence of malpositioning such as in an inadequate vessel or having caused injury.

Side-entry jackets and diversion jackets require postioning through a small incision; however, there is then no question about a need for means of confirmation to rule out mispositioning or injury (see, for example, Ablordeppey, E. A., Drewry, A. M., Beyer, A. B., Theodoro, D, L., Fowler, S. A., Fuller, B. M., and Carpenter, C, R. 2017. “Diagnostic Accuracy of Central Venous Catheter Confirmation by Bedside Ultrasound Versus Chest Radiography in Critically Ill Patients: A Systematic Review and Meta-Analysis,” Critical Care Medicine 45(4): 715-724; Wilson, S. P., Assaf, S., Lahham, S., Subeh, M., Chiem, A., and 4 others 2017. “Simplified Point-of-care Ultrasound Protocol to Confirm Central Venous Catheter Placement: A Prospective Study,” World Journal of Emergency Medicine 8(1): 25-28; Tomaszewski, K. J., Ferko, N., Hollmann, S. S., Eng, S. C., Richard, H. M., Rowe, L., and Sproule, S. 2017. “Time and Resources of Peripherally Inserted Central Catheter Insertion Procedures: A Comparison Between Blind Insertion/Chest X-ray and a Real Time Tip Navigation and Confirmation System,” Clinicoeconomics and Outcomes Research 9:115-125; Bidgood, C. 2016. “Improving the Patient Experience with Real-time PICC Placement Confirmation,” British Journal of Nursing 25(10): 539-543; Kamalipour, H., Ahmadi, S., Kamali, K., Moaref, A., Shafa, M., and Kamalipour, P. 2016. “Ultrasound for Localization of Central Venous Catheter: A Good Alternative to Chest X-Ray?,” Anesthesiology and Pain Medicine 6(5): e38834; Dale, M., Higgins, A., and Carolan-Rees, G. 2016. “Sherlock 3CG(®) Tip Confirmation System for Placement of Peripherally Inserted Central Catheters: A NICE [National Institute for Health and Care Excellence of Britain] Medical Technology Guidance,” Applied Health Economics and Health Policy 14(1): 41-49; Roldan, C. J. and Paniagua, L. 2015. “Central Venous Catheter Intravascular Malpositioning: Causes, Prevention, Diagnosis, and Correction,” Western Journal of Emergency Medicine 16(5): 658-664; Duran-Gehring, P. E., Guirgis, F. W., McKee, K. C., Goggans, S., Tran, H., Kalynych, C. J., and Wears R. L. 2015. “The Bubble Study: Ultrasound Confirmation of Central Venous Catheter Placement,” American Journal of Emergency Medicine 33(3): 315-319; Gekle, R., Dubensky, L., Haddad, S., Bramante, R., Cirilli, A., and 7 others 2015. “Saline Flush Test: Can Bedside Sonography Replace Conventional Radiography for Confirmation of Above-the-diaphragm Central Venous Catheter Placement?,” Journal of Ultrasound in Medicine 34(7): 1295-1299; Brass, P., Hellmich, M., Kolodziej, L., Schick, G., and Smith, A, F. 2015. Ultrasound Guidance versus Anatomical Landmarks for Internal Jugular Vein Catheterization,” Cochrane Database of Systematic Reviews 2015 1: CD006962; Brass, P., Hellmich, M. Kolodziej, L., Schick, G., and Smith, A, F. 2015. “Ultrasound Guidance versus Anatomical Landmarks for Subclavian or Femoral Vein Catheterization,” Cochrane Database of Systematic Reviews 1: CD011447; Cortellaro, F., Mellace, L., Paglia, S., Costantino, G., Sher, S., and Coen, D. 2014. “Contrast Enhanced Ultrasound vs Chest X-ray to Determine Correct Central Venous Catheter Position,” American Journal of Emergency Medicine 32(1): 78-81; Vezzani, A., Brusasco, C., and Corradi, F. 2014. “Contrast-enhanced Ultrasound to Determine Correct Central Venous Catheter Position,” American Journal of Emergency Medicine 32(7): 809-810; Heran, M. K. and Burrill, J. 2012. “Vascular Pediatric Interventional Radiology,” Canadian Association of Radiologists Journal 63(3 Supplement): S59-S73; Krishnamurthy, G. and Keller, M. S. 2011. “Vascular access in Children,” Cardiovascular and Interventional Radiology 34(1): 14-24; Vezzani, A., Brusasco, C., Palerm, S., Launo, C., Mergoni, M., and Corradi, F. 2010. “Ultrasound Localization of Central Vein Catheter and Detection of Postprocedural Pneumothorax: An Alternative to Chest Radiography,” Critical Care Medicine 38(2): 533-538; Matsushima, K. and Frankel, H. L. 2010. “Bedside Ultrasound Can Safely Eliminate the Need for Chest Radiographs after Central Venous Catheter Placement: CVC Sono in the Surgical ICU (SICU),” Journal of Surgical Research 163(1): 155-161). Clotting may be more pronounced during plasmapheresis, or plasma exchange.

With medication delivered through a singular line, doses intended for local and systemic application cannot be suitably apportioned. Either a dose intended exclusively for local application that would best be constrained to the target segment continues, albeit progressively more and more diluted as it passes through the systemic circulation, to reach other tissue which it can adversely affect, or a strong initial dose intended for greater concentration within a restricted zone and lower concentration throughout the balance of the systemic circulation becomes too dilute. When the medication should be circulated, the systemic concentration should not be dictated by that of the local dose, and the local dose should not be constrained by the dose intended to pass into the systemic circulation.

Provided reversal agents are available, positioning a second line and side-entry jacket downstream from the first allows the intervening segment to be substantially isolated for proper dosing with the appropriate drug or drugs. In fact, three zones are created, that preceding the first line, that between the first and second lines, and that following the second line. This is because both lines can feed both drugs and reversal agents for drugs other than each delivers. Thus, a drug targeting the cutdown or puncture site in higher concentration and intended for uptake only there and over the segment between the two lines can be completely or partially neutralized at the second line.

Drugs for delivery at higher concentration at and over a short segment following the cutdown or puncture site include an anticoagulant and an anti-inflammatory. In a patient prone to clotting (see, for example, Jaffray, J., Bauman, M., and Massicotte, P. 2017. “The Impact of Central Venous Catheters on Pediatric Venous Thromboembolism,” Online, Frontiers in Pediatrics 5:5; Schoot, R. A., van de Wetering, M. D., Stijnen, T., Tissing, W. J., Michiels, E., Abbink, F. C., Raphael, M. F., and 5 others 2016. “Prevalence of Symptomatic and Asymptomatic Thrombosis in Pediatric Oncology Patients with Tunneled Central Venous Catheters,” Pediatric Blood and Cancer 63(8): 1438-1444; Sapienza, S. P. and Ciaschini, D, R. 2015. “Intraluminal Volume Dose Alteplase for the Clearance of Occluded Peripherally Inserted Central Catheter Lines at a Long-Term Acute Care Hospital: Efficacy and Economic Impact,” Hospital Pharmacy 50(3): 202-207; Anderson, D. M., Pesaturo, K. A., Casavant, J., and Ramsey, E. Z. 2013. “Alteplase for the Treatment of Catheter Occlusion in Pediatric Patients,” Annals of Pharmacotherapy 47(3): 405-409) or one made susceptible to clotting as the direct result of placing the line, a lesser concentration of the anticoagulant should continue through the circulation.

In a patient with normal clotting factors, placement of the lines with a ductus side-entry jacket or diversion side-entry jacket is less promoting of clotting, because there is no object suspended in the native lumen as would cause turbulent flow, and placement is less traumatizing. That reversal agents must not themselves introduce significant problems is superfluous. The realization that an anticoagulant can be infused along with chemotherapeutics, for example, so as to preclude the formation of thrombus has been implemented, but not as to allow automatic delivery of both anticoagulant and reversal agent in an ambulatory patient with an implanted central line, such as a central venous catheter, running from a body surface port, reservoir, catheter, and through a side-entry jacket (see, for example, Brandão, L. R., Shah, N., and Shah, P. S. 2014. “Low Molecular Weight Heparin for Prevention of Central Venous Catheterization-related Thrombosis in Children,” Cochrane Database of Systematic Reviews(3): CD005982).

While a conventional portacath is adequate for the infusion of one or more drugs directly into the superior vena cava or the jugular or subclavian vein, for example, an automatic disorder response system that requires the targeted injection or infusion of different drugs into different ductus in order to treat comorbid conditions in a coordinated manner would require multiple portacaths, creating the need for a compact subdermal port with multiple injection and/or infusion entry points. Deviations from normal renal and micturative function, to include diagnostic monitoring and response, can be incorporated into such an automatic system.

When used to connect a central line for a purpose other than temporary such as to release a drug or parenteral nutrition systemically, a simple flow diversion side-entry jacket such as those shown in U.S. Pat. No. 11,759,186, entitled Ductus Side-entry Jackets and Prosthetic Disorder Response Systems, can be placed at any level along the vessel where the jacket will be unobtrusive and not cause the patient discomfort. This will usually mean the inferior rather than the superior vena cava or the abdominal rather than the thoracic aorta. Such a jacket is internally configured to provide a fixed chute similar to that retractable and extendable in a vascular valve for redirecting flow out the jacket side stem.

The jacket terminus to a central line or peripherally inserted central catheter used to tale blood-draw, blood transfusion entry, drugline most often for antibiotics, intravenous fluids, and chemotherapy, for example, can be positioned at any level along the ductus to which the drug or nutritional substance is to be targeted and/or a sensor is to be positioned. When the device is not part of an implanted prosthetic disorder response system under the control of an implanted microcontroller or microprocessor, the ‘dumb’ entry can be a conventional subcutaneously implanted Mediport® or Portacath®. Compared to a peripherally inserted central catheter, the latter are superior for long-term use at the entry point, and while it requires placement thorough a small incision, for use over an indeterminate length of time, the use of a side-entry diversion jacket at the terminus makes this end equally safe and secure.

2a(5). Vascular Access

Unlike an indwelling catheter, using a body surface port and connector of the kind described herein, a central line with a subcutaneous port much smaller than a conventional port at the proximal end connected by a ductus side-entry diversion jacket to the target ductus, typically a major vein in the upper chest, at the distal end, is stabilized and secured as not to limit the activities in which the patient can participate.

Placement of the line avoids the more intricate surgery necessary to create an arteriovenous fistula for the attachment of a hemodialysis machine, for example. Any invasive procedure risks complications such as persistent irritation and infection. Conventional means for placing such a line are more susceptible to complications, such as the development of large bulging varicose veins on the inner surface of the lower arm, needless injury, and infection, than are the means shown herein.

Other than through a central line, ‘vascular access’ refers to surgically created arteriovenous fistulas and grafts. For simple applications, such as vascular access for hemodialysis or apheresis which can be used immediately, or a central line which conventionally may be removed and replaced repeatedly as might an indwelling catheter, ductus side-entry jackets eliminate the surgery required to create an arteriovenous fistula or complications associated with the connection of synthetic lines to native ductus and allow freedom of movement without risk of injury even when the device is to remain indwelling for an extended period if not permanently. Moreover, once placed, if then removed and reinserted, the line and its connection at either end are prone to irritation and infection, and the terminus to clot.

Vascular access is addressed below in this section, and an implanted magnetic aphereter/dialyzer described in copending application Ser. No. 18/358,252, entitled Fully Implanted Aphereter/Dialyzers for Intracorporal Blood Purification, referred to below in section 2b(2)(h)(h2), entitled The ‘Tuned’ Shunt in the Relief of Portal Venous Hypertension, is described in U.S. Pat. No. 11,759,186, entitled Ductus Side-entry Jackets and Prosthetic Disorder Response Systems, and shown in FIGS. 39A and 39B of that application. This device releases the extracted analyte or analytes into a line filled with dialysate, thence, directly into the urinary bladder, if desired, for examination through urinalysis. Since extraction is selective at the molecular or particular level, no other components of the blood are removed or need to be returned.

With this magnetic aphereter/dialyzer, no surface port, central line, or vascular access in the form of a venous catheter, arteriovenous fistula, or arteriovenous graft is needed; however, a small port such as shown in FIG. 26A or FIG. 45B in the alternative reference cited, positioned subcutaneously anywhere on the body surface allows direct access to any artery or vein. Referring to FIG. 39B in copending application Ser. No. 15/998,002, replacement of the dialysate is thru a small dedicated port, part number 120 with lines 97 and 99 with incurrent and excurrent openings positioned subcutaneously in the mons pubis.

Here the double-cartridge-loading manual devices for replenishing the dialysate shown in FIGS. 27A and 27B can be used to inject two different solutions, the existing fill if any allowed to passively spill out of either hole in the surface port, or the larger of the two compartments can be used to collect the used dialysate as that smaller is used for replenishment. The same means can be used to add medication or a solvent added to the dialysate or separately into the dialysate line 79.

In FIG. 39B of copending application Ser. No. 15/998,002, access to the dialysate line 79 can also be gravity fed or pumped automatically from a subcutaneously positioned body surface port in the pectoral region thru a drugline connected to the line under the control of an implanted microcontroller, or in comorbid disease, a microprocessor. Depending upon the analyte or analytes, meaning the solutes, toxins, or blood components to be extracted, the device can be used alone and without modification, in another embodiment to incorporate a more conventional dialysate and flush-cleanable filtration membrane in the same unit, or can be used concurrently with conventional dialysis in a clinic.

Despite using an extraction interface with a much smaller cross-section than those found in nonelectrolytic laboratory dialysis or apheresis machines, because 1. Rather than through passive dialysis, the magnetic device works by active attraction, is 2. Supplied with power through transcutaneous energy transfer, and therefore, 3. Can function as long as necessary, the rate of extraction is still functional, certainly sufficient to extend the period between vistis to the clinic significantly. Control of the process can be automatic, sensors for evaluating the need for blood purification signaling the need to the implanted prosthetic disorder response system addressed above in section 2a, entitled Background of the Invention. General.

Using conventional hemodialysis with the port shown in FIG. 26A, the other difference is that distal connection to the target vessel or vessels is through small ductus side-entry jackets likewise shown in the alternative reference cited. With more than one injection opening, the subcutaneous surface access port can be used for another or an allied purpose, it can be satisfied with a small body surface port with a distributor such as shown in FIG. 26B can be used to directly pipe-target drugs, solutions, or solvents, for example, to any point in the body.

Unrelated to the implanted magnetic device, a small subsutaneously implanted port can connect to any vein and artery, not those small in the forearm, to access a larger flow, hence, and quicker analyte extraction rate. To emplace the magnetic device requires an invasive procedure, after which no further invasive measures are needed. Eliminating is the irritation and risk of infection that a surgically created arteriovenous fistula, for example, poses with every visit to the clinic. Vascular access is addressed both in its own right and as a factor in immune tolerance induction in preparation for an organ transplant by reducing donor-recipient incompatibility to reduce the odds for rejection.

Vascular accesses and central lines are uninvolved in the reciprocal cross-circulation used to effect organ transfer from a donor to a recipient. Connections between corresponding vessels such as depicted in FIG. 16 and in the direct aortocaval connection through a confluence conduit shown in FIG. 17B is through bloodlines connected at either end by vascular valves or servovalves. In FIG. 17B (and FIGS. 22C thru 22F), the drugline to the inlet side-entry vascular servovalve is shown as part number 8, while the drugline to the outlet servovalve is shown as part number 8′, and flow that continues through the substrate vessel is shown as part number 41, while that diverted is shown as part number 41′.

When the immune profile of the prospective donor and recipient prompt, and time to prepare for a metered solid organ transplant allows, a series of visits to the clinic may justify a vascular access or central line to facilitate preparatory immune tolerance induction using drugs and/or gene therapy, for example, addressed above in section 2a, entitled Background of the Invention. General. The objects in use of an implantable dialysis device are to extend the period of time between visits to the clinic or in simple cases, eliminate that need.

Such treatment as incorporated into the operation itself is through the accessory channels of the valves. With the implanted magnetic aphereter/dialyzer, extraction is from the inferior vena cava with its higher flow rate for release directly into the urinary bladder. Depending upon the selective extractability of the analyte as bonded to its superparamagnetic iron oxide nanoparticulate carrier, this may limit use to treatment pertaining to only one or a few analytes at a time.

The incorporation into the magnetic aphereter/dialyzer of a solvent flush cleanable filtration membrane for an analyte or analytes to be extracted for a specific patient allows combined ambulatory use, but the need for another type membrane will require treatment in a clinic. This is in contrast to a clinic which has apparatus that allows blood purification processes to be performed concurrently. Where successive use with this ambulatory device eliminates the need for the patient to appear at the clinic for a 2-4 hour visit every two weeks, if more than one process is needed, the clinic can reduce the overall time for processing, but at the inconvenience of the patient.

In the clinic, hemodialysis and hemapheresis can be performed in tandem, that is in series or parallel. “Simultaneous use of multiple modalities with dedicated machines combined in series or in parallel is defined as tandem therapies,” (Pekkucuksen, N. Sigler, K. E., AkcanArikan, A., and Srivaths, P. 2021. “Tandem Plasmapheresis and Continuous Kidney Replacement Treatment in Pediatric Patients,” Online, Pediatric Nephrology (Berlin, Germany) 36(5): 1273-1278)(see also, Foglia, M. J., Pelletier, J. H., Bayir, H., Fleck, A., Konyk, L., and 3 others 2022. “Tandem Therapeutic Plasma Exchange Reduces Continuous Renal Replacement Therapy Downtime,” Online, Blood Purification 51(6): 523-530; Kaushik, M., Liew, Z. H., Sewa, W., Phua, G. C., Cao, L., and 7 others 2021. “Description of Parallel and Sequential Configurations for Concurrent Therapeutic Plasma Exchange and Continuous Kidney Replacement Therapy in Adults,” Journal of Clinical Apheresis 36(1): 211-218; Kaplan, A, A. and Balogun, R, A. 2020. “Therapeutic Plasma Exchange (Plasmapheresis with Hemodialysis Equipment,” Online, uptodate.com; Sirignano R. M., Meyer, E. K., Fasano. R., and Paden, M. L. 2018. “Pediatric Tandem Therapeutic Apheresis: A Multidisciplinary Approach,” American Society for Artificial Internal Organs Journal 64(3): 382-388; Eding, D. M., Jelsma, L. R., Metz, C. J., Steen, V. S., and Wincek, J. M. 2011. “Innovative Techniques to Decrease Blood Exposure and Minimize Interruptions in Pediatric Continuous Renal Replacement Therapy,” Critical Care Nurse 31(1): 64-71; Bhowmik, D., Jain, P. K., Masih, J. A., Saha, D., Gupta, S., and 3 others 2001. “Tandem Plasmapheresis and Hemodialysis,” Therapeutic Apheresis and Dialysis 5(5): 439-441). Simultaneous connection in parallel is necessary when the procedural duration does not afford the time for use in succession or to establish and test two separate vascular accesses.

Hypothetically, at least, since extracorporeal hemapheresis, and sometimes requiring additional equipment, photopheresis accomplish functions which depending upon the condition of the patient may be complementary, both types of apheresis, plasmapheresis and cytapheresis, for example, can be connected successively or in parallel through separate venous access anastomoses or bilateral Power Flow ports, or in series through the same venous access anastomosis or Power Flow port, then through standardized connectors in series to more thoroughly extract immunologically conflicting components in the blood of the donor and recipient (see, for example, Knobler, R., Berlin, G., Calzavara-Pinton, P., Greinix, H., Jaksch, P., and 35 others 2014. “Guidelines on the Use of Extracorporeal Photopheresis,” Online, Journal of the European Academy of Dermatology and Venereology 28 (Supplement 1): 1-37).

Guidance on the use of photopheresis to reduce if not eliminate the risk of acute and chronic graft-versus-host disease, chronic lung allograft dysfunction, bronchiolitis obliterans syndrome, restrictive allograft syndrome, and impairment in breathing due to gastroesophageal reflux disease and elucidation as to its function is readily available (see, for example, Bedair, B. and Hachem, R, R. 2021. Op cit., third paragraph above, section on extracorporeal photopheresis; Braun, L. M. and Zeiser, R. 2021. “Immunomodulatory Therapies for the Treatment of Graft-versus-host Disease,” Online, HemaSphere (Philadelphia, Pennsylvania) 5(6);e581, section on extracorporeal photopheresis; Crocchiolo, R., Cesana, C., Girgenti, D., Bertani, G., Barba, C., and 6 others 2021. “Tregs and GvHD Prevention by Extracorporeal Photopheresis: Observations from a Clinical Trail,” Experimental Hematology and Oncology 10(1): 14, erratum 10(1): 22; Dieterlen M. T., Klaeske, K., Bernardt, A. A., Borger, M. A., Klein, S., and 5 others 2021. “Immune Monitoring Assay for Extracorporeal Photopheresis Treatment Optimization after Heart Transplantation,” Online, Frontiers in Immunology (Lausanne, Switzerland) 12:676175; Benazzo, A., Worel, N., Schwarz S., Just. U., Nechay, A., and 9 others 2020. “Outcome of Extracorporeal Photopheresis as an Add-on Therapy for Antibody-mediated Rejection in Lung Transplant Recipients,” Transfusion Medicine and Hemotherapy 47(3): 205-213; Drexler. B., Buser, A., Stehle, G., Halter, J., and Holbro, A. 2020. “Extracorporeal Photopheresis in Graft-versus-Host Disease,” Transfusion Medicine and Hemotherapy 47(3): 214-225; Padmanabhan, A., Connelly-Smith, L. Aqui, N., Balogun, R. A., Klingel, R., and 8 others 2019. “Guidelines on the Use of Therapeutic Apheresis in Clinical Practice-Evidence-based Approach from the Writing Committee of the American Society for Apheresis: The Eighth Special Issue,” Journal of Clinical Apheresis 34(3): 171-354; Worel. N. Taleghani, B. M., and Strasser E. 2019. Op cit., third paragraph above; Cho, A., Jantschitsch, C., and Knobler, R. 2018. “Extracorporeal Photopheresis—An Overview,” Online, Frontiers in Medicine (Lausanne, Switzerland) 5:236, sections on graft versus host disease and solid organ transplantation; Moniodis, A., Townsend, K., Rabin, A., Aloum, O., Stempel, J. and 9 others 2018. “Comparison of Extracorporeal Photopheresis and Alemtuzumab for the Treatment of Chronic Lung Allograft Dysfunction” Journal of Heart and Lung Transplantation (New York, New York) 37(3): 340-348; Robinson, C. A., Huber, L., Murer, C., Schuurmans, M., Kohler, M., Hofbauer, G., and Benden, C. 2017. “Cessation of Extracorporeal Photopheresis in Chronic Lung Allograft Dysfunction: Effects on Clinical Outcome in Adults,” Swiss Medical Weekly 147: w14429; Mortzell, H. M., Newman, E., Witt. V., Derfler. K., Leitner, G., and 34 others 2016. “Adverse Events in Apheresis: An Update of the WAA [World Apheresis Association] Registry Data,” Transfusion and Apheresis Science (Oxford, England) 54(1): 2-15; Greinix, H. T., Worel, Nand., Just. U., and Knobler, R. 2014. “Extracorporeal Photopheresis in Acute and Chronic Graft-versus-host Disease,” Transfusion and Apheresis Science (Oxford, England) 50(3): 349-357; Knobler, R. Berlin, G., Cazavara-Pinton, P. Greinix, H., and Jaksch, P., and 34 others 2014. “Guidelines on the Use of Extracorporeal Photopheresis,” Journal of the European Academy of Dermatology and Venereology 28 (Supplement 1): 1-37; Greinix, H. and Knobler, R. 2012. Extracorporeal Photopheresis, Berlin, Germany and Boston, Massachusetts: Walter de Gruyter GmBH: Jaksch, P. Scheed. A., Keplinger, M. Emst. M. B., Dani, T., and 4 others 2012. “A Prospective Interventional Study on the Use of Extracorporeal Photopheresis in Patients with Bronchiolitis Obliterans Syndrome after Lung Transplantation,” Journal of Heart and Lung Transplantation (New York, New York) 31(9): 950-957; Wong. E, C. C/and Jacobsohn, D. 2012. “ECP [extracorporeal photopheresis] in Children and Adolescents,” in Greinix, H. and Knobler, R. 2012. Op cit.; Knippen, M, A. 2011, Op. cit., fourth paragraph above; Hannani. D., Gabert, F., Laurin, D., Sall. M., Molens J. P., and 3 others 2010. “Photochemotherapy Induces the Apoptosis of Monocytes without Impairing Their Function,” Transplantation 89(5): 492-499: Garza, E., Rogers, C. E., Clouther, S. G., Lowler, K. P. Tawara, I., and 3 others 2008. “Extracorporeal Photopheresis Reverses Experimental Graft-versus- host Disease through Regulatory T Cells,” Blood 112(4): 1515-1521; Ibrahim, R. B., Liu. C., Cronin, S. M., Murphy, B. C., Cha, R., Swerdlow. P., and Edwards, D. J. 2007. “Drug Removal by Plasmapheresis: An Evidence-based Review,” Pharmacotherapy 27(11): 1529-1549; Legitimo, A., Consolini, R., Failli, A., Fabiano, S., Bencivelli, W., Scatena, F. and Mosca, F. 2007. “In Vitro Treatment of Monocytes with 8-methoxypsolaren and Ultraviolet A Light Induces Dendritic Cells with a Tolerogenic Phenotype,” Clinical and Experimental Immunology 148(3): 564-572; Spisek, R., Gasova, Z., and Bartunkova, J. 2006. “Maturation State of Dendritic Cells during the Extracorporeal Photopheresis and Its Relevance for the Treatment of Chronic Graft-versus-host Disease,” Transfusion 46(1): 55-65).

Extracorporeal hemapheresis and/or photopheresis can be used as necessary both pre- and post- as well as midprocedurally to dispel acute and chronic graft-versus-host disease (see, for example, Yerrabothala, S., Talebian, L., Klinker, K., Hickman, J., Hill, H. M., and 4 others 2018. “Extracorporeal Photopheresis for Graft versus Host Disease: Identifying a Clinical Pathway and Associated Resource Utilization,” Journal of Clinical Apheresis (Hoboken, New Jersey) 33(3): 310-315; Azar, N., Leblond, V., Ouzegdouh, M and Button, P, A. 2017. “A Transition from Using Multi- step Procedures to a Fully Integrated System for Performing Extracorporeal Photopheresis: Comparison of Costs and Efficiencies,” Journal of Clinical Apheresis 32(6): 474-478; Greinix, H. T., Worel. N., Just. U., and Knobler, R. M. 2014. “Extracorporeal Photopheresis in Acute and Chronic Graft-versus-Host Disease,” Transfusion and Apheresis Science 50(3): 349-357; Marques. M. B., Singh, H., and Reddy, V. V. 2014. “Out with the Bad and in with the Good: Red Cell Exchange, White Cell Reduction, and Platelet Reduction,” Journal of Clinical Apheresis 29(4): 220-227; Weitz, M., Strahm, B., Meerpohl, J. J., and Bassler, D. 2014. “Extracorporeal Photopheresis versus Alternative Treatment for Chronic Graft-versus-host Disease after Haematopoietic Stem Cell Transplantation in Paediatric Patients,” Online, Cochrane Database of Systematic Reviews (Chichester, West Sussex, England) (2): CD009898; Chih. S., Tinckam, K. J., and Ross, H. J. 2013; “A Survey of Current Practice for Antibody-mediated Rejection in Heart Transplantation,” American Journal of Transplantation 13(4): 1069-1074; Klassen, J. 2010. “The Role of Photopheresis in the Treatment of Graft-versus-Host Disease,” Current Oncology 17(2): 55-58; Baird, K. and Wayne, A. S. 2009. “Extracorporeal Photo-apheresis for the Treatment of Steroid-resistant Graft versus Host Disease,” Transfusion and Apheresis Science 41(3): 209-218; Greinix, H. T., Knobler, R. M., Worel. N., Schneider, B., Schneeberger, A., and 5 others 2006. “The Effect of Intensified Extracorporeal Photochemotherapy on Long-term Survival in Patients with Severe Acute Graft-versus-Host Disease,” Haematologia 91(3): 405-408).

Hygienic measures to prevent infection during insertion are no different for any other type of vascular access (see, for example, Al Mokali, K., Al Sannaa, Z., Al Mutairi, F., and Ahmed, A. E. 2020. “Factors Influencing Occurrence of Peritonitis in Saudi Children on Peritoneal Dialysis,” Online, BioMed Central Pediatrics (London, England) 2020 20(1): 42; Fisher, M., Golestaneh, L., Allon, M., Abreo. K., and Mokrzycki, M, H. 2020. “Prevention of Bloodstream Infections in Patients Undergoing Hemodialysis,” Clinical Journal of the American Society of Nephrology 15(1): 132-151; Marsenic, O., Rodean, J., Richardson, T., Swartz, S., Claes, D., and 3 others 2020. “Tunneled Hemodialysis Catheter Care Practices and Bloodstream Infection Rate in Children: Results from the SCOPE [Standardizing Care to Improve Outcomes in Pediatric End Stage Renal Disease] Collaborative,” Pediatric Nephrology (Berlin, Germany) 35(1): 135-143; Alhazmi, S. M., Noor, S. O., Alshamrani, M. M., and Farahat, F. M. 2019. “Bloodstream Infection at Hemodialysis Facilities in Jeddah: A Medical Record Review,” Annals of Saudi Medicine 39(4): 258-264; Bademler, S., Üçüncü, M., Yιldιrιm, i., and Karanlιk, H. 2019. “Risk Factors for Complications in Cancer Patients with Totally Implantable Access Ports: A Retrospective Study and Review of the Literature,” Journal of International Medical Research 47(2): 702-709; He. Y., Zhao, H., Wei. Y., Gan, X., Ling. Y., and Ying. Y. 2019. “Retrospective Analysis of Microbial Colonization Patterns in Central Venous Catheters, 2013-2017,” Journal of Healthcare Engineering 2019:8632701; Lukito, A. A., Pranata, R., Huang. I., Thengker, A., and Wirawan, M. 2019. “Fracture of the Port Catheter and Migration into the Coronary Sinus: Case Report and Brief Review of the Literature,” Clinical Medicine Insights. Case Reports (Auckland, New Zealand) 12:1179547619832282; Machat. S., Eisenhuber, E., Pfarl, G., Stubler, J., Koelblinger. C., Zacherl. J., and Schima W. 2019. “Complications of Central Venous Port Systems: A Pictorial Review,” Insights into Imaging 10(1): 86; Poinen, K., Quinn, R. R., Clarke, A., Ravani. P., Hiremath, S., and 3 others 2019. “Complications from Tunneled Hemodialysis Catheters: A Canadian Observational Cohort Study,” American Journal of Kidney Diseases 73(4): 467-475; Chalhoub. M., Saqib, A., and Castellano, M. 2018. “Indwelling Pleural Catheters: Complications and Management Strategies,” Journal of Thoracic Disease (Hong Kong, China) 10(7): 4659-4666; Nelveg-Kristensen, K. E., Laier, G. H., and Heaf, J, G. 2018. “Risk of Death after First-time Bloodstream Infection in Incident Dialysis Patients with Specific Consideration on Vascular Access and Comorbidity,” Online, BioMed Central Infectious Diseases (London, England) 18(1): 688; Pinelli, F., Cecero, E., Degl′Innocenti, D., Selmi. V., Giua. R., and 4 others 2018. “Infection of Totally Implantable Venous Access Devices: A Review of the Literature,” Journal of Vascular Access (Thousand Oaks, California) 19(3): 230-242: Yagnik, L. and Shrestha, R. 2018. “Spontaneous Fracture of Indwelling Pleural Catheter,” Respirology Case Reports 6(8): e00365; Zadeh, M. K., Esfahani, A. D., and Azadeh, A. 2018. “Dislocation of the End of the Catheter Due to Pressure of the Mass of Soft Tissue of the Patient's Breast,” Journal of Vascular Access (Thousand Oaks, California) 19(1): 106-107; Assis, L. V., Pereira, E. R., Sa, C. B., Guimarães-Neto, I., Masson-Silva, J. B., and 4 others 2017. “Right Ventricular Perforation by Tunneled Hemodialysis Catheter,” Kidney International 91(1): 255; Nguyen, D. B., Shugart, A., Lines, C., Shah, A. B., Edwards, J., and 3 others 2017. “National Healthcare Safety Network (NHSN) Dialysis Event Surveillance Report for 2014,” Clinical Journal of the American Society of Nephrology 12(7): 1139-1146; Shenep. M. A., Tanner, M. R., Sun, Y., Culley, T., Hayden, R. T., and 3 others 2017. “Catheter-related Complications in Children with Cancer Receiving Parenteral Nutrition: Change in Risk Is Moderated by Catheter Type,” Journal of Parenteral and Enteral Nutrition 41(6): 1063-1071; Wasse, H. 2017. “Cerebral Hyperperfusion and Other Consequences of Hemodialysis Central Vein Catheters,” Journal of Vascular Access (Thousand Oaks, California) 18 (Supplement 1): 82-83; Houston, B. L. and Yan, M. 2016. “Spontaneous Migration of an Implanted Central Venous Access Device into the Ipsilateral Jugular Vein,” Canadian Medical Association Journal 188(10): 752; Ko. S. Y., Park, S. C., Hwang, J. K., and Kim, S, D. 2016. “Spontaneous Fracture and Migration of Catheter of a Totally Implantable Venous Access Port via Internal Jugular Vein-A Case Report,” Journal of Cardiothoracic Surgery 11:50; Ma, L. I., Liu, Y., Wang, J., Chang. Y., Yu, L., and Geng, C. 2016. “Totally Implantable Venous Access Port Systems and Associated Complications: A Single-institution Retrospective Analysis of 2.996 Breast Cancer Patients,” Molecular and Clinical Oncology 4(3): 456-460; Sethna, C. B., Bryant, K., Munshi, R., Warady, B. A., Richardson, T., and 3 others 2016. “Risk Factors for and Outcomes of Catheter-associated Peritonitis in Children: The SCOPE [Standardizing Care to Improve Outcomes in Pediatric End Stage Renal Disease] Collaborative,” Clinical Journal of the American Society of Nephrology 11(9): 1590-1596; An, H., Ryu, C. G., Jung, E. J., Kang, H. J., Paik, J. H., Yang. J. H., and Hwang, D. Y. 2015. “Insertion of Totally Implantable Central Venous Access Devices by Surgeons,” Annals of Coloproctology (Seoul, South Korea) 31(2): 63-67; Bohlke, M., Uliano, G., and Barcellos, F, C. 2015. “Hemodialysis Catheter-related Infection: Prophylaxis, Diagnosis, and Treatment,” Journal of Vascular Access (Thousand Oaks, California) 16(5): 347-355; Granziera, E., Scarpa, M., Ciccarese, A., Filip. B., Cagol. M., and 6 others 2014. “Totally Implantable Venous Access Devices: Retrospective Analysis of Different Insertion Techniques and Predictors of Complications in 796 Devices Implanted in a Single Institution,” Online, BioMed Central Surgery (London, England) 14:27; Kulkami, S., Wu, O., Kasthuri, R., and Moss, J, G. 2014. “Centrally Inserted External Catheters and Totally Implantable Ports for the Delivery of Chemotherapy: A Systematic Review and Meta-analysis of Device-related Complications,” Cardiovascular and Interventional Radiology 37(4): 990-1008; Seok, J. P., Kim, Y. J., Cho, H. M., Ryu, H. Y., Hwang. W. J., and Sung, T. Y. 2014. “A Retrospective Clinical Study: Complications of Totally Implanted Central Venous Access Ports,” Korean Journal of Thoracic and Cardiovascular Surgery 47(1): 26-31; Capitanini, A., Ricci, E., Frosini, P., and Cupisti, A. 2013. “Massively Calcified Intravascular Cast after Removal of a Tunneled Central Vein Catheter for Hemodialysis,” Journal of Vascular Access (Thousand Oaks, California) 4(2): 196-198; Keum, D. Y., Kim, J. B., and Chae. M, C. 2013. “Safety of a Totally Implantable Central Venous Port System with Percutaneous Subclavian Vein Access,” Korean Journal of Thoracic and Cardiovascular Surgery 46(3): 202-207).

Unlike conventional subcutaneously positioned ports, the distal end of the much smaller multiple opening ports addressed here are securely connected to the target ductus by means of a side-entry jacket or valve jacket. In contrast, the distal end of conventional ports having been known to migrate causing serious complications (see, for example, Intagliata. E., Basile, F., and Vecchio, R. 2017. “Totally Implantable Catheter Migration and Its Percutaneous Retrieval: Case Report and Review of the Literature,” Giornali de Chirurgia 37(5): 211-215; Pikija, S., Pilz, G., Gschwandtner, G., Rösler, C., Schlick, K., Greil, R., and Sellner, J. 2016. “Panitumumab-associated Encephalopathy after Accidental Intra-arterial Application Through Dislocated Central Venous Access Device,” Online, Frontiers in Neurology 7:196; Struck, M. F., Junemann, T., Reinhart, K., and Schummer, W. 2016. “Central Venous Catheter Tip Migration Due to Tracheal Extubation: A Prospective Randomized Study,” Journal of Clinical Monitoring and Computing July 28 (in press); Prabaharan, B. and Thomas, S. 2014. “Spontaneous Migration of Central Venous Catheter Tip Following Extubation,” Saudi Journal of Anaesthesia 8(1): 131-133; Shah, B. K., Srijan Tandukar, S., Shrestha, S., and Sanchirico, P. 2014. “A Rare Case of Port-a-Cath Migration into the Mediastinum,” West Indian Medical Journal 63(6): 676-677; Wu. P. Y., Yeh, Y. C., Huang, C. H., Lau, H. P., and Yeh, H. M. 2005. “Spontaneous Migration of a Port-a-Cath Catheter into Ipsilateral Jugular Vein in Two Patients with Severe Cough” Annals of Vascular Surgery 19(5): 734-736), a side-entry jacket at the distal end of a conventional portacath would overcome this problem. Because the foam lining complies with dynamic changes in diameter of the substrate ductus, so that the ductus side-entry jackets to which each subport of the small multiport at the body surface is piped remains stationary in encircling relation to the peristaltic ureter or pulsatile blood vessel.

The stable connection of the catheter to the vessel by the jacket ends the risks of dislodgement, fracture, and fragmentation experienced with conventional methods of insertion, to include the sticking of long-term hemodialysis catheters following fibrous entrapment (see, for example, Kalińczuk, Ł., Chmielak, Z., Dębski, A., Kępka, C., Rudzinski, P. N., and 5 others 2016. “Percutaneous Retrieval of Centrally Embolized Fragments of Central Venous Access Devices or Knotted Swan-Ganz Catheters. Clinical Report of 14 Retrievals with Detailed Angiographic Analysis and Review of Procedural Aspects,” Postepy Kardiol Interwencyjnej [Advances in Interventional Cardiology] 12(2): 140-155; Lodi, M., Ebrahimi, R. F., Pezzotti, P., and Carbonari, L. 2016. “The Removal of a Stuck Catheter: An Alternative to Hong's Technique,” Journal of Vascular Access (Thousand Oaks, California) 17(6): 548-551; Adeel Ebad, C., Davitt, S., Gnanasekaran, R., Khan, A., and Moran, A. M. 2016, Op cit.; Vellanki, V. S., Watson, D., Rajan, D. K., Bhola, C. B., and Lok, C, E. 2015, Op cit.” Hong, J, H. 2011. “A Breakthrough Technique for the Removal of a Hemodialysis Catheter Stuck in the Central Vein: Endoluminal Balloon Dilatation of the Stuck Catheter,” Journal of Vascular Access (Thousand Oaks, California) 12(4): 381-384; Hong, J, H. 2010. “An Easy Technique for the Removal of a Hemodialysis Catheter Stuck in Central Veins,” Journal of Vascular Access (Thousand Oaks, California) 11(1): 59-62; Chuang, M. T., Wu, D. K., Chang, C. A., Shih, M. C., Ou- Yang, F., and 3 others 2011. “Concurrent Use of Pigtail and Loop Snare Catheters for Percutaneous Retrieval of Dislodged Central Venous Port Catheter,” Kaohsiung Journal of Medical Sciences 27(11): 514-519; Al-Hadi, H. and Sallam, M. 2009. “Retrieval of Dislodged Central Venous Pressure Catheters: Early Experience with Three Case Presentations,” Sultan Qaboos University Medical Journal 9(2): 175-179).

As shown in FIGS. 10, 11, and 12, with donor or recipient inverted, either one, or two-way (bidirectional) valves can be connected to divert flow to or from the same direction in either the donor or recipient vessel into either direction in the donor or recipient vessel, and with donor and recipient uninverted, bidirectional valves can be used to divert flow to or from the opposite direction in either the donor or recipient vessel into either direction in the donor or recipient vessel.

However, combinations of same and opposite diversion so that the two substreams are caused to converge above the upper diversion chute or below the lower diversion chute are discouraged as causing increased pressure and hemolytic injury (Vercaemst, L. 2008. “Hemolysis in Cardiac Surgery Patients Undergoing Cardiopulmonary Bypass: A Review in Search of a Treatment Algorithm,” Journal of Extracorporeal Technology (Les Ulis Cedex, France) 40(4): 257-267).

Diversion thus is from one artery to another or from one vein to another, not from one to the other; crossover from the one to other type vessel effectively reproduces the problems of mixing oxygenated with unoxygenated blood seen in congenital defects of the cardiovascular system, and following surgical arteriovenous fistulation to create vascular access for the attachment of a hemodialysis machine, which results in large bulging varicose veins on the inner surface of the lower arm. Placement of a small manual bidirectional valve to create an arteriovenous fistula for hemodialysis vascular access connects the vessels with reversal in the direction of flow with less dissection and less risk of infection than does an anastomosis.

Synthetic tubing not susceptible to “ . . . inflammation, uremia, hypoxia, sheer stress, and increased thrombogenicity’ leading to intimal hyperplasia, another advantage of takeoff at the ureters using synthetic materials is avoidance of the procedural risks and the 40 percent obstruction due to intimal hyperplasia experienced at one year following arteriovenous fistulation, (or U.S. fistulization, British fistulisation) (see, for example, Brahmbhatt, A., Remuzzi, A., Franzoni, M., and Misra, S. 2016. “The Molecular Mechanisms of Hemodialysis Vascular Access Failure,” Kidney International 89(2): 303-316; Lee, T. and Misra, S. 2016. “New Insights into Dialysis Vascular Access: Molecular Targets in Arteriovenous Fistula and Arteriovenous Graft Failure and Their Potential to Improve Vascular Access Outcomes,” Clinical Journal of the American Society of Nephrology 11(8): 1504-1512).

2b. Vascular Valves and Servovalves in the Diversion of Blood Flow

Blood diversion valves make possible the seamless, uninterrupted, and hypoxia-free direct transfer of a solid organ from the circulatory system of the donor into that of the recipient by anoxia-free bypass, or sudden switched-organ, transplantation, and the mid- and/or postprocedural bypass of defects in the vascular tree. Medical conditions spectral in requiring treatment comprehending numberless variables, the means described are intended to serve as alternative or adjuvant in relation to established measures for the treatment of disease which is serious and chronic, not acute and responsive to conventional treatment.

All side-entry devices are provided with multiple means for avoiding irritation to the substrate vessel as might cause these to swell and interfere with flow. These include a lining designed to minimize compression and irritation, apertures to avoid completely enclosing the adventitia, now known to be atherogenic, and a drugline to allow anti-inflammatory, antithrombotic, antimicrobial, and other medication to be directly targeted into the jacket or into the jacket-ductus interface.

Side-entry diversion jackets which can be internally configured to divert a fixed fraction of the flow through the substrate ductus into a branch or alternative passageway are addressed in U.S. Pat. No. 11,759,186, entitled Ductus Side-entry Jackets and Prosthetic Disorder Response Systems. In contrast, solenoid-driven side-entry vascular valves are bistably limited to diverting all or none of the flow into a shunt or bypass, making these suitable for sudden compound vascular bypass, or switched-organ transplantation, and side-entry vascular servovalves are continuously variable in the fraction of flow-allowed to pass without diversion and that diverted.

An endoluminal diverter is limited to diverting all flow through it into a synthetic shunt or bypass. To assure servovalve displacements are as commanded and incapable of deviating under higher pressures, the armature is notched at intervals along its length and a detent consisting of a prominence along its bore, or channel, provided to hold the armature in place at the position commanded, a relatively stronger power surge commanded by the implanted controller, signaled the deviation from the instant set point, then used to controllably dislodge and move it. Servochokes intended for positioning along lines subject to higher pressures are likewise provided this means for assuring positional stabilization and preventing the possibility of a complete closure causing ischemia.

Without certain materials and the use of anticoagulants to prevent the accumulation of clot in valves and passageways, crystal solvents to prevent the accretion of crystal and antimicrobials respectively along the internal walls of the passageways and valves, a system consisting of relatively small caliber tubes and valves would soon become impassable. FIGS. 22B and 32F show how in the event of an occlusion, anastomosis bypass pathways can provide an alternative path around such a stoppage.

Aberrant inputs originating at the system sensors make the need for diversion immediately evident to the system master control microprocessor, which causes the bypass inlet and outlet valves to institute diversion. To assure passageways will be free of obstruction, newer parts coatings and embedded substances are employed, and thrombophobic, antimicrobial, and crystal formation-counteractive solutions are made to slowly drip through valve accessory channels. Obstruction counteractive measures are addressed below.

### One-, Two-, and Three-Way Passive Side-Entry Flow Diversion Jackets, or Flow Directional Guides

Defined as a device for controlling the passage of a fluid through a pipe, a passive side-entry flow diversion jacket as a nonadjustable flow directional guide which not a valve complements an adjustable valve in continuing to guide flow through a prescribed passageway. Lacking an adjustability or valving function, flow diversion side-entry jackets with one, two, and three flow diversion passages as shown in FIGS. 10F, 10G, and 10H respectively are simple and relatively inexpensive less but versatile than adjustable manual and solenoid vascular valves and servovalves.

That shown in FIG. 10F is a one-way simple flow diversion side-entry jacket used to direct a singular influent or entering fluid column, while the two-way simple flow diversion jacket shown in FIG. 10G accepts a separate inflow column through either side. Inverted, the latter can be used to divide or split a single column into two outlet channels. Lacking a protrusive sidestem and more costly, where only the direction of flow is required, by eliminating the time to find the best level and angle to avoid encroachment on neighboring tissue, a side-entry flow diversion jacket facilitates placement in compact tissue. In FIG. 28, the prosthetic urinary collection system shown provides for valves 143 and 143′ as one-way directional flow guides.

Because in this prosthesis is meant for a patient without the function normally provided by the parts of the urinary tract replaced, no valving function is necessary. The use of valves here is to facilitate implantation of the prosthesis by allowing the operator to use manually operated valves as shown in FIG. 2 to switch between the flow-open and flow-closed position during placement. Placement and removal of either device is by insertion or withdrawal through the side entry hole produced with the aid of a catheter with razor sharp ferrule tip attached to a suction pump to draw out a plug of tissue, using the thumb to stop any bleeding.

The opening is then taped over with an absorbable surgical tape having a dissolution time exceeding that for the opening to close in secondary intention. These passive devices are often adequate for use at the effluent end of a bloodline entered into and a more costly servovalve at the opposite entry end as a volume of flow-through controlling as well as directing guide into the receiving vessel. A third type simple flow diversion side-entry jacket shown in FIG. 10H accepts three input flow streams.

These passive devices are often adequate for use at the effluent end of a bloodline entered into through a more costly servovalve as a volume of flow-through controlling as well as directing guide into the receiving vessel. A third type simple flow diversion side-entry jacket shown in FIG. 10H accepts three input flow streams. The two-way passive side-entry flow diversion jacket shown in FIG. 10G is used in the application depicted in FIG. 15B to seamlessly transfer an allograft heart which having been transplanted using the compound vascular bypass method has been added to the defective native heart where both hearts have been kept separate so that each is intact.

With the continuous and immediate medicinal, and if necessary, synchronization support provided by the implanted automatic response system, the possibility for the restoration to adequate function of the native heart-even following a conventional anastomotic transplant-exists. However, now a chimera comprised of allogeneic hearts which have been anastomosed, are not disengageable, and upon whose continued function the life of the recipient depends, the possibility for transfer of the portion of the added heart from the first to a second recipient does not exist.

However, if and when the separate and intact native heart following a compound vascular bypass transplant recovers to explant the intact added intact heart for transfer to a second recipient becomes a realistic possibility to further increase the pool of available hearts. This practical eventuality imparted by the support of a fully implanted prosthetic disorder response system and compound vascular bypass heart transplantation is addressed below in section 2b(1)(b)(b5), entitled Heterotopic Double Heart Metered Compound Vascular Bypass, or Switched-organ Transplantation [and so on], in the subsection thereof entitled Seamless and Ischemia-free Explantation and Transfer of an Allograft Second Heart from a First to a Second Recipient.

Used thus, the two-way arterial and venous passive side-entry flow diversion jackets at the center to supply and drain the allograft reduce the cost and complexity of the overall blood flow circuit. This is because flow through the center is passively guided by the servovalves at either end. When the procedure commences, the valves on the right are halfway extended to pass flow through the center into the allograft while the valves on the left are retracted to cut off flow to and from the allograft. The right and left half-circuits can therefore be used independently without the need for adjustability at the center.

FIG. 15B is schematic in showing the hearts, each about the same size as the closed first of the recipient, reduced to postage stamp size in relation to the upper arterial and lower venous two-way flow diversion jackets, actually about the same size as small sewing thimbles, used to allow the gradual transfer of flow from an allograft second heart to assist that which had been failing but then recovered ejection fraction sufficiency or other solid organ implanted in a first recipient to a second recipient, thus switching the organ from the circulatory system of the first recipient into that of the second. In this application where flow through the allograft is shared in turn with either native heart, the sum of both inflows is equal to the full volume of blood to pass through either pair.

Like vascular valves and servovalves, these simple flow diversion side-entry jackets comprise 1. A plano-hinged jacket made of an implantable material such as polyetheretherketone of polypropylene consisting of two antipodal half cylinders with a closing snap opposite the hinge, 2. A lining of viscoelastic polyurethane foam perforated to the extent that the continuous portions of the jacket represent a smaller total area of the lattice-like jacket with through-and-through round or diamond-shaped holes which continue in depth from the exterior surface of the jacket down to the adventitia, and a central hole to admit a diversion chute entered through a sidestem, and 3. A diversion chute with entry side-stem and accessory channel to allow the insertion of a drugline led from a drug reservoir implanted subcutaneously, usually in the pectoral region.

Alternative methods for diverting flow from one ductus into another requiring transecting the vessel necessitating bypass, cardioplegia, and interruption in normal flow, the use of a simple side-entry jacket avoids the need for the interruption in circulation through the affected part and the need for cardiopulmonary bypass as the major source of mid- and postoperative complications. The central object in side-entry devices is to eliminate all of these complications. In a simpler implanted automatic disorder response system, control over the release of medication or a maintenance solution such as a calcium oxalate solvent through a side-entry jacket is administered by an implanted microcontroller through a drugline 8 inserted into an accessory channel in the jacket.

In FIG. 15B, the direct gradual transfer of the donor heart at the center of the figure from the first recipient on the right, to the second recipient, on the left, with no interruption in blood flow requires that blood through the donor heart be gradually and continuously switched from the first to the second recipient. To do this, all connections between the donor organ and those of the recipient to either side must be established prior to initiating the switching operation. Accordingly, the nonadjustable, or simple, double side-entry flow diversion jacket shown in FIG. 10G channels the continuously transferred increase in flow volume received from the first to the second recipient.

In FIG. 15B, the vascular valves at the four corners of the figure gradually transfer the perfusion of the donor organ between them. At the commencement of the operation, the servovalves in the right-hand corners of the drawing figure are at the setting that had been used to perfuse the donor heart while it was implanted in and shared perfusion with that of the recipient. If the hearts were the same in size, this would have been with both the arterial flow control valve at the top right and the venous flow control valve at the bottom right with their flow diversion chutes at half-way extension.

As the operation progresses, the servovalves to the right are gradually closed in synchrony with the complementary opening of those to the left, so that as the valves on the right gradually close, those on the left gradually open, thus causing the sharing of perfusion with the graft organ to transfer from the first to the second recipient. The valves on the right will completely close at the same time that those on the left are fully opened. The passive two-way diversion jackets exerting no control function, flow up through the central bloodline from the venous jacket at the bottom and the arterial jacket at the bifurcation at the top pass through a constant volume of blood through the graft organ at the center. Its composition is such that the relative proportion of the volume contributed by the first recipient to the right is initially exclusive and gradually reduced as the volume contributed by the second recipient to the left is synchronously increased.

In a metered, that is, nonemergency, switched organ operation as would normally to be expected, the donor, or graft organ and both recipients must be immunologically evaluated prior to the operation, potential complications accounted for in advance in a contingency plan by loading the system drug reservoirs with the counteractants indicated. Three-way crossmatching is essential whenever a recipient must be implanted with an organ from one donor and another organ from another donor and is determined by crossmatching each in turn. Transfer of the donor heart from the first to the second recipient is under the control of the implanted automatic disorder system, which controls the servovalves to include appropriate adjustments thereto based upon signs of rejection signaled by the implanted sensors.

If detected, the control system directly pipe-targets reversal medication to either or both the donor and recipient heart as detected from the sensors. Allograft organ rejection sensors are addressed above in section 2a, entitled Background of the Invention. General, in the subsection thereof entitled Sensors, part. e, entitled Graft Organ Rejection Analytes and Immunosensors. Such dosing can be gradually adjusted from the initial to the second recipient-donor pairing as the action progresses. Once perfusion of the donor heart has been transferred from the first to the second recipient, the bloodlines connecting the donor heart to that of the first recipient are removed and the insertion openings in the passive three-way valves plugged shut, any seepage of blood readily cleared with the aid of an aspirator.

In all three types of passive, or simple, flow diversion jackets, the need for a more costly mechanism to adjust the degree of extension of the diversion chute 18, which is fused to the external jacket 3 is obtained by making the closing snap incremental and the foam lining 4 thick enough so that the operator can use an ultrasound probe to view the degree of penetration of the tip of the diversion chute into the intimal lining of the substrate vessel through the apertures 40 in the jacket 3. To obturate the lumen of the ductus with its feathered surround edge when unfolded, the diversion chute, slightly larger in diameter than the ostium, is made of a durable thrombophobically treated rubbery material such as a medical-grade silicone elastomer easily pinched narrower by the operator to allow its insertion through the ostium and into the lumen of the substrate vessel.

As indicated, such a nonadjustable diversion jacket is often suitable at the outlet of a bloodline entered through a vascular valve or servovalve for the purpose of continuing flow at the volume set by the vascular servovalve at the entry end through the effluent ductus, usually a vessel, in the direction required. Such a nonadjustable diversion jacket is positioned along a vessel, for example, by snapping it shut at the snap closing device increment and level along the vessel desired. The distal tip of the catheter with a razor-sharp ferrule annulus connected to an aspirator is inserted into the hole through the sidestem 19 against the outer layer of the vessel wall 2, or adventitia.

Turning on the aspirator then draws the sharp end of the catheter through the wall of the ductus and extracts a plug from the vessel wall. As soon as this ostium, or perforation into the lumen of the vessel 1 is complete, the operator places his thumb over the opening to prevent extravasation or leakage. Diversion chute 18 is unitized with the sidestem behind it which latter has a lumen through it to allow the insertion of a mainline, usually a bloodline, to exit at the upper surface of the entry end of the diversion chute. The diversion chute with sidestem is passed through the opening, or ostium, into the substrate vessel lumen.

For exceptional temporary placement, the surfaces of both the mainline insertion opening and the distal end of the mainline are scored to securely fasten the line in place in a friction fit (pressed, interference) joint. The diversion chute is permanently fixed in position to pass through the maximum volume of flow admitted by the valve at the entry into the mainline, thus accommodating any lesser volume. Fed through bloodlines connected to it on either side, the bilateral inflow separator embodiment shown in FIG. 10G is unable to accommodate the full volume of both; however, the use of this device involves the exchange in passage of the donor heart half-volume between the recipients.

If and when either is the one or two-sided device to be removed, the sidestem with unitized diversion chute is pulled out of the ostium, the operator again using his thumb to stop the bleeding or loss of other contents such as a hormone, for example. The bilateral double outflow separator embodiment shown in FIG. 10G and the triple out flow separator embodiment shown in FIG. 10H are placed as is the single inflow embodiment shown in FIG. 10F, the operator then using the suction catheter to draw out a plug on the opposite side of the substrate vessel. A turn of a surgical tape having an absorption time keyed to the natural filling in of the ostium by second intention is wrapped around the ostium.

For other than the application indicated but rather an exceptional permanent placement, the surfaces of both the mainline insertion opening and the distal end of the mainline are scored to securely fasten the line in place in a friction fit joint made permanent by wetting the scored surfaces with a suitable cyanoacrylate cement. As indicated, the bilateral double and triple inflow diversion jackets shown in FIGS. 10G and 10H are inserted in the same way as that unilateral, the second sidestem on the opposite side requiring the use of the aspiration catheter to extract it, the operator again using a finger to prevent bleeding.

2b(1). The Intercorporeal Diversion of Blood Flow

By extracorporeal vascular diversion is meant the directly piped intercorporeal exchange of blood or blood products between patients, almost always the donor and recipient during the transplantation of one or more solid organs. The term extracorporeal also applies to an exchange of blood between a patient and a machine, whether an artificial heart, cardiopulmonary bypass machine, ex vivo perfusion machine, or extracorporeal membrane oxygenator used as a bridge pending the availability of a suitable transplant organ. In the harvesting of an unpaired organ, the donor will be deceased having been placed on life support prior to having died.

This seamless and anoxia-free transfer from matched donor to recipient and the continuous mid- and postprocedural support by a totally implanted disorder response system that had administered the transfer and thereafter serves to detect the need for and automatically dispense medication to include immunosuppressive immediately is likely to ward off posttransplant complications such as non-melanoma basal or squamous cell or melanoma skin cancer, and the development of late-term cardiac allograft vasculopathy, which latter is the most frequent cause for late-term failure of a heart transplant.

The increased risk for cancer after an organ transplant has long been known (see, for example, Gogna, S., Ramakrishna, K., and John, S. 2023. “Posttransplantation Cancer,” Online, Treasure Island, Florida: StatPearls Publishing Co; Spetsotaki, K., Koch., A, Taube, C., Theegarten, D., Kamler, M., and Pizanis, N. 2023. “Incidence of Malignancies after Lung Transplantation and Their Effect on the Outcome. 26 Years' Experience,” Online, Heliyon (London, England) 9(10): e20592; Oerlemans, M. I, F. J., Groenewegen, G., Vink, A., van Laake, L. W., van der Kaaij, N. P., and de Jonge, N. 2021. “Donor-derived Testicular Germ Cell Cancer in a Heart Transplant Recipient,” Online, Journal of the American College of Cardiology CardioOncology (New York, New York) 3(2): 322-325; Ge F, Li C, Xu X, Huo Z, Wang R, and 14 others 2020. “Cancer Risk in Heart or Lung Transplant Recipients: A Comprehensive Analysis of 21 Prospective Cohorts,” Online, Cancer Medicine (Malden, Massachusetts) 9(24): 9595-9610; Huo Z, Li C, Xu X, Ge F, Wang R, and 14 others 2020. “Cancer Risks in Solid Organ Transplant Recipients: Results from a Comprehensive Analysis of 72 Cohort Studies, Online, Oncoimmunology (Philadelphia, Pennsylvania) 9(1): 1848068; Krynitz, B., Edgren, G., Lindelof, B., Baecklund, E., Brattstrom, C., Wilczek, H., and Smedby, K, E. 2013. “Risk of Skin Cancer and Oher Malignancies in Kidney, Liver, Heart and Lung Transplant Recipients 1970 to 2008—A Swedish Population-based Study,” Online, International Journal of Cancer (New York, New York) 132(6): 1429-1438).

The compound vascular bypass method improves solid organ transplantation in two principal respects: the anoxic pumpless transfer of the organ directly from the donor kept on life support after having died into the recipient, and the targeted delivery of medication such as chemotherapeutic, antimicrobial, anti-inflammatory, and immunosuppressive not only during but following the transplant. These make an implanted automatic disorder response system no less significant for its administration of the transplant than for the postoperative follow-up protection it provides, likely extending the life of an organ recipient for many years.

As to terminology, in switching from the longer to the shorter bloodline in an individual long-to-short bloodline device as described below in section 2b(1)(b)(b13), entitled Bloodline and Drugline Switch Devices: Long when Intercorporeal to Short when Intracorporeal to Accommodate Growth, or from a longer to a shorter bloodline confluence conduit in a multiple bloodline switch device as described below in section 2b(1)(b)(b15), entitled Switchable Lines and Confluence Conduits: Long when Intercorporeal, to Short when Intracorporeal, Both Affording Connection of One of More Bloodlines to the Same or a Different Number of Bloodlines with Self-leveling Apportionment of Outflow, and shown schematically in FIG. 35B, the process of a switch, or compound vascular bypass, transplantation transitions from intercorporeal between the donor and recipient to intracorporeal in the recipient.

In a compound vascular bypass switch transplant, described and illustrated in sections to follow, the donor and recipient are positioned side by side, their blood exchanged by directly interconnecting piping. There is no hard, or “bright line,” distinction between the use of different type tie-lines as between those ‘anatomical’ prostheses which emulate the conformation of the structure to be replaced such as the aortic arch with supraaortic branches and those catheteric or tubular.

For example, longer bloodlines and longer switchable confluence conduits are usually used intercorporeally during a compound vascular bypass, or sudden switched-organ organ transplant, but can also be used to connect extracorporeal equipment and be reinserted after a previously implanted line had become outgrown. Both individual, long-to-short or the reverse switchable lines and switchable confluence conduits cross over the distinction between either inter- or intra-corporeal; rather, these begin as intercorporeal, usually during the performance of a compound vascular bypass, or sudden switched-organ solid organ transplantation and end as intracorporeal when the procedure culminates in the placement of the graft organ in the recipient using only the shorter of the two switch lines or confluence conduits.

If later due to growth the longer line must be reconnected, that is accomplished endoscopically without any involvement of another party and remains intracorporeal. Both the native and the replacement organ remain completely intact with all arteries and veins unaffected until the replacement organ must be harvested. Due to the inadequacy of available graft organs, the compresence of the donor and recipient will often be unfeasible. Rather than continued on life support past death with donor and recipient brought to the same center, the donor will already have died with graft organs harvested and stored in a static or flowing chilled perfusate or maintained with the aid of an ex vivo perfusion machine.

Processing thus not preferred but as a practical matter, often unavoidable, it is important that to expedite diversion valve placement, the arteries and veins of the graft organ when harvested be transected to leave the stumps as long as possible. As used herein, intracorporeal means inside the body; paracorporeal means worn on the body, such as the power pack for a ventricular assist device or a urine collection bag; and extracorporeal means apparatus separate from but connected to the body. In switch transplantation, blood flow between the recipient and donor is extracorporeal until the donor organ is harvested and placed in the recipient, whereupon flow is intracorporeal in the recipient.

In a sudden compound vascular bypass, or sudden switched-organ, transplant, a vascular solenoid, or in a metered switch transplant, a vascular servovalve is actually needed only for the blood supply to both the donor and recipient organs, additional valves used when helpful in adjusting the local blood pressure or to gain immediate proximity to the substrate ductus for the release of drugs through the valve accessory channel or channels. If both recipient a donor organs are to remain as in a double kidney or double heart transplant, then because it is continuously variable as to the volume of blood which can be diverted and apportioned between the two, a servovalve must be used. Blood supply and venous drainage can be mediated by basic side-entry jackets incorporating a permanently fully open diversion chute.

Diversion in the present context means redirecting a portion of the flow into a larger vessel, usually through a vascular diversion servovalve controlled by an implanted microcontroller—or in multimorbid disease, a control microprocessor—and should not be confused with flow diverters or flow diversion stents used to treat intracranial saccular aneurysms (see, for example, Chiu, A, H. Y. and Phillips, T. J. 2020. “Future Directions of Flow Diverter Therapy,” Neurosurgery 86 (Supplement 1): S106-S116; Wu, X., Tian, Z., Liu, J., Zhang, Y., Li, W., and 5 others 2019. “Patency of Posterior Circulation Branches Covered by Flow Diverter Device: A Hemodynamic Study,” Online, Frontiers in Neurology 10:658; Clarencon, F., Di Maria, F., Gabrieli, J., Shotar, E., Zeghal, C., and 3 others 2017. “Flow Diverter Stents for the Treatment of Anterior Cerebral Artery Aneurysms: Safety and Effectiveness,” Clinical Neuroradiology 27(1): 51-56).

Also functionally distinct is high flow bypass to treat large aneurysms of the internal carotid, for example (see, for example, Mohri, M., Ichinose, T., Uchiyama, N., Misaki, K., Nambu, I., Takabatake, Y., and Nakada, M. 2018. “Hyperperfusion Syndrome after Trapping with High-flow Bypass for a Giant Paraclinoid [location opposite the clinoid process] Internal Carotid Artery Aneurysm,” World Neurosurgery 115:143-146; Nakajima, N., Nagahiro, S., Satomi, J., Tada, Y., Nakajima, K., and 5 others 2015. “Prevention of Retrograde Blood Flow into Large or Giant Internal Carotid Artery Aneurysms by Endovascular Coil Embolization with High-flow Bypass: Surgical Technique and Long-term Results,” World Neurosurgery 2015 83(6): 1127-1134).

A compound vascular bypass, or sudden switched-organ, transplant is exceptional in requiring 18-21 vascular servovalves. Other solid organ transplants require relatively few valves for a sudden or a metered compound vascular bypass, or sudden switched-organ transplant. Exactitude in the positioning of vascular valves and servovalves along the arterial supply and venous drainage and the angle thereof is noncritical. Therefore, when the arteries and veins of the organ are degraded as well, and/or the valves when placed in the recipient would encroach upon neighboring tissue, the organ of the donor is removed with a sufficient length of its arteries and veins to afford latitude in the positioning of the valves. Such pertains to the transplantation of any solid organ using either the sudden or metered switch method addressed below, wherein the heart is used as exemplary.

To avoid abrasive contact, the valves and servovalves have rounded corners, edges, and provided with a soft polymeric covering to minimize any negative consequence of contact with neighboring tissue. If not compressive, contact with neighboring tissue can usually be disregarded in a sudden compound vascular bypass, or sudden switched-organ transplant which absent any unanticipated complications, is completed in relatively little time. In a metered switch transplant which might extend over a period of days, potential damage to donor vessels warrants carefully positioning of the servovalves much as in the recipient to avoid encroachment.

A diversion jacket for placement about a ureter does not pose the problem of forcible expulsion when the effluent opening is cut in the side of the ductus and does not require an accessory channel with a water jacket-configured distal terminus at the ductus end. A diversion jacket trepan end-piece must incorporate both a diversion chute and a water jacket. To accomplish this, the diversion chute is placed inside the water jacket, thus creating a side-entry diversion valve jacket.

This reduces the diameter of the opening through which the chute must retract, causing its sides to become frayed if frequently retracted past the trepan. Measures to eliminate side fraying of the chute include dulling the trepan edge at the sides and metalizing, such as aluminizing, without changing the elasticity of the chute side edges. Since the trepan can be rotated during placement, and the circumference of the chute affected is insignificant, side dulling is harmless. If the sides of a chute with conventional trepan end-piece should fray, the remedy is the same. Because a vascular side-entry diversion jacket is placed only about larger vessels, the limitation on miniaturization imposed should never emerge as a problem. Due to the intrinsic motility of a ureter and traveling wave of the pulse through an artery, a diversion jacket for placement along such ductus must incorporate effective means for preventing distal migration as specified below.

Unlike subcutaneously or subdermally placed body surface ports, where first the surrounding fat and palpation projections hold the port in position, and depending upon the location, the development by the body of a fibrous capsule to contain the foreign object would reinforce the stabilization, diversion jackets are positioned about ductus, where neither may be present to a sufficient degree. It is therefore essential to provide means for preventing migration. When the likelihood of migration is slight, as when the jacket is held in place by surrounding fat, ancillary means for fixing the jacket at the level placed are omitted. Two options for resisting migration can be used for wearers who are refractory or acutely sensitive to alternative measures.

Outlined below, for example, will be a description of the sudden compound vascular bypass, or sudden switched-organ technique as applied to a heart transplant from a fetal donor recently lost due to factors other than a malformed heart to another fetus that would be lost due to a heart so malformed that its optimal repair would not likely yield normal circulation much less a life span beyond the first decade. This circumstance is addressed for several reasons:

1. The intravascular side-entry diversion valves to be described make possible solid organ, to include heart transplantation by sudden switching of the recipient's circulatory system from his innate to the donor organ.

2. Gradual or metered switching transfers the graft organ from the donor to the recipient through a continuous process of replacement by monitored degrees to allow more extended, perhaps even optimized immune tolerance induction.

3. This capability extends to fetuses with extensively malformed hearts for whom the alternative to transplantation would be lengthy and traumatic complex intracardiac microsurgery incapable of providing a successful outcome beyond mere survival.

4. The younger the donor and recipient, the less developed are the immune systems, lessening the risks of organ rejection and graft versus host disease and the need for chimerizing measures at the outset, greater amenability to, these measures facilitating post transplantation maintenance.

5. Transplantation not suggested for genuinely repairable defects, no repair of more complex congenital heart defects however skilled achieves normal circulatory function, surgical remediation often properly relegated to a bridging procedure until a replacement heart can be found.

6. So long as development continues with a badly defective heart, the lack of normal circulation is propagated throughout the body as a chronic hypoxia that results in progressive multiple, indeed global, organ damage, severely affecting the brain, liver, and kidneys, and a life limited by the continuous and inexorable increase in left ventricular load that comes with growth and adds to the antecedent accumulation of damage until the dysfunction becomes greater than the impaired heart can withstand.

7. Immediate replacement of a badly defective heart in utero or shortly postpartum averts operative and postoperative complications which can result from preceding attempts at repair, to include obscuring scarring, adhesions, and the delay in a good correction while damage caused by faulty circulation continues to accumulate, significantly improving the prospects for successful transplantation (see, for example, Alsoufi, B., Mahle, W. T., Manlhiot, C., Deshpande, S., Kogon, B., McCrindle, B. W., and Kanter, K. 2016. “Outcomes of Heart Transplantation in Children with Hypoplastic Left Heart Syndrome Previously Palliated with the Norwood Procedure,” Journal of Thoracic and Cardiovascular Surgery 151(1): 167-175.e1-e2; Schumacher, K, R. and Goldberg, D. J. 2016. “Biomarkers and the Fontan Circulation,” Journal of the American Heart Association (Oxford, England) 5(1). pii: e002926; Lang. S. M., Frazier, E. A., and Collins, R. T. 2nd 2016. “Aortic Complications following Pediatric Heart Transplantation: A Case Series and Review,” Annals of Pediatric Cardiology 9(1): 42-45; Alsoufi, B., Deshpande, S., McCracken, C., Kogon, B., Vincent. R., Mahle, W., and Kanter, K. 2015. “Results of Heart Transplantation following Failed Staged Palliation of Hypoplastic Left Heart Syndrome and Related Single Ventricle Anomalies,” European Journal of Cardiothoracic Surgery 48(5): 792-799, also cited below; Kanter, K. R., Mahle, W. T., Vincent, R. N., and Berg, A. M. 2011. “Aortic Complications after Pediatric Cardiac Transplantation in Patients with a Previous Norwood Reconstruction,” Seminars in Thoracic and Cardiovascular Surgery. Pediatric Cardiac Surgery Annual 14(1): 24-28).

8. Transplantation yields the greatest lifelong benefit, critically superior to an imperfect repair that does not initiates normal circulation. Automatic dispensing of anti-rejection medication does away with the need for the patient to remember when the next dose is due and limits dose size to minimize the risk of susceptibility to infection. That using the means described herein, immunosuppressant, antimicrobial, and anti-inflammatory drugs can be automatically and directly targeted to the transplant substantially diminishing the quality of life reducing need for the patient to administer these to himself.

9. Fetal and neonatal transplantation of the heart complex, the application of parallel procedural steps for the transplantation of other solid organs will be evident without elaboration. Much of the complexity is, however, immunological, and a procedure in some respects even more complex, a liver transplant from a donor on life support, is addressed below.

10. The critical reduction in procedural complexity, eliminating the demand for the broad experience, physiological acumen, and skill required for optimal diagnosis and reconstruction where the deviations from normal are spectral allows a local surgeon to operate as a ‘parts changer’ without the astuteness essential to perform a complex internal organ repair inside a tiny heart, and without the need to anastomose the tiny vessels immediately, the ability required to deal with a complex surgical problem is lessened to a degree that such procedures can be performed at local hospitals without the risk of critical time loss involved in transfer to a quaternary center. Unlike confronting a tiny, uniquely malformed heart that requires the utmost astuteness to repair to an optimal extent that must fall short of providing normal circulation anyway, the procedures described herein are substantially invariant and consistent with the need for standardization. This at a time when immunosuppressant understanding is expanding to minimize the burden of transplant drug maintenance and avoid its numerous side effects as a key factor in the avoidance of transplantation in the first place.

11. Another factor that allows more local application is that the prescription-program in support of the procedure, which demands much experience, much less expertise to optimize, can be prepared by highly experienced subspecialized internists and hematologists in large centers for dissemination to practitioners at lower patient volume institutions, detailed tailoring to a specific patient supported with guidance available from the same or another group of centralized experts.

Objections asserting the irrecoverability of transvenous or coronary sinus leads with the aid of a standard stylet after more than a year and the disallowance of magnetic resonance imaging are negated, first by awareness as to techniques for making possible the safe extraction of retained transvenous lead fragments of hearts since removed for transplantation (Leung, L. W. M., Gomes, J., Domenichini, G., and Gallagher, M. M. 2019. “Oesophageal Perforation: An Unexpected Complication during Extraction of a Pacing Lead. A Case Report,” European Heart Journal. Case Reports 3(1): ytz008; Younis, A., Glikson, M., Meitus, A., Arwas, N., Natanzon, S. S., and 4 others 2019. “Transvenous Lead Extraction with Laser Reduces Need for Femoral Approach during the Procedure,” Online, PLoS [Public Library of Science] One 14(4): e0215589; Azarrafiy, R. and Carrillo, R, G. 2018. “Surgical and Hybrid Lead Extraction,” Cardiac Electrophysiology Clinics 10(4): 659-665; Elshazly, M, B. and Tarakji, K, G. 2018. “Reimplantation after Lead Removal,” Cardiac Electrophysiology Clinics 10(4): 667-674; Hasumi. E., Fujiu, K., Kojima, T., Kinoshita, O., Nawata. K., and 3 others 2018. “Novel Extraction Technique of Retained Pacemaker and Defibrillator Lead during Heart Transplantation,” Online, PLoS [Public Library of Science] One 13(9): e0203172; Krainski, F., Pretorius, V., and Birgersdotter-Green, U. 2018. “A Practical Approach to Lead Removal: Transvenous Tools and Techniques,” Cardiovascular Electrophysiology Clinics 10(4): 637-650; Menezes Junior, A, D. S., Magalhães, T. R., and Morais, A. O, A. 2018. “Percutaneous Lead Extraction in Infection of Cardiac Implantable Electronic Devices: A Systematic Review,” Brazilian Journal of Cardiovascular Surgery 33(2): 194-202; Migliore, F., Cavalli, G., Bottio. T., Testolina, M., De Lazzari, M., and 3 others 2018. “Hybrid Minimally Invasive Technique with the Bidirectional Rotational Evolution® Mechanical Sheath for Transvenous Lead Extraction: A Collaboration between Electrophysiologists and Cardiac Surgeons,” Journal of Arrhythmia 34(3): 329-332; Love, C. J. 2018. “Lead Management and Lead Extraction,” Cardiac Electrophysiology Clinics 10(1): 127-136; Okada, A., Shoda, M., Tabata, H., Shoin, W., Kobayashi, H., and 6 others 2018. “Single-center Experience with Percutaneous Lead Extraction of Cardiac Implantable Electric Devices,” Journal of Cardiology 71(2): 192-196; Perez, A. A., Woo, F. W., Tsang, D. C., and Carrillo, R, G. 2018. “Transvenous Lead Extractions: Current Approaches and Future Trends,” Arrhythmia and Electrophysiology Review 7(3): 210-217; Bontempi, L., Vassanelli, F., Cerini, M., Bisleri, G., Repossini, A., and 8 others 2017. “Hybrid Minimally Invasive Approach for Transvenous Lead Extraction: A Feasible Technique in High-risk Patients,” Journal of Cardiovascular Electrophysiology 28(4): 466-473; Cronin, E. M. and Wilkoff, B. L. 2017. “Coronary Sinus Lead Extraction,” Heart Failure Clinics 13(1): 105-115; Domenichini, G., Gonna, H., Sharma, R., Conti, S., Fiorista, L., and 6 others 2017. “Non-laser Percutaneous Extraction of Pacemaker and Defibrillation Leads: A Decade of Progress,” Europace 19(9): 1521-1526; Pecha. S., Linder, M., Gosau, N., Castro, L., Vogler, J., and 3 others 2017. “Lead Extraction with High Frequency Laser Sheaths: A Single-centre Experience,” European Journal of Cardiothoracic Surgery 51(5): 902-905: Pecha. S., Castro, L., Vogler, J., Linder, M., Gosau, N., and 3 others 2018. “Differences in Laser Lead Extraction of Infected vs. Non-infected Leads,” Heart and Vessels 33(10): 1245-1250; Pecha, S., Yildirim, Y., Gosau, N., Aydin, M. A., Willems. S., and 3 others 2017. “Laser Lead Extraction Allows for Safe and Effective Removal of Single- and Dual-coil Implantable Cardioverter Defibrillator Leads: A Single-centre Experience over 12 Years,” Interactive Cardiovascular and Thoracic Surgery 24(1): 77-81; Goya, M., Nagashima, M., Hiroshima, K., Hayashi, K., Makihara, Y., and 12 others 2016. “Lead Extractions in Patients with Cardiac Implantable Electronic Device Infections: Single Center Experience,” Journal of Arrhythmia 32(4): 308-312; Jo. U., Kim, J., Hwang. Y. M., Lee, J. H., Kim, M. S., and 8 others 2016. “Transvenous Lead Extraction via the Inferior Approach Using a Gooseneck Snare versus Simple Manual Traction,” Korean Circulation Journal 46(2): 186-196; Wazni, O. and Wilkoff, B. L. 2016. “Considerations for Cardiac Device Lead Extraction,” Nature Reviews. Cardiology 13(4): 221-229; Maciąg. A., Syska, P., Sterlinski, M., Przybylski, A., Sitkowska, E., and 4 others 2015. “Lead extraction: The Road to Successful Cardiac Resynchronization Therapy,” Cardiology Journal 22(2): 188-193; Okamura, H. 2015. “Editorial: What Makes Transvenous Extraction More Difficult?,” Journal of Cardiology Cases 13(1): 31-32; Ribeiro, S., Leite, L., Oliveira, J., Pereira, M. J., Pinheiro, C., and 6 others 2015. “Transvenous Removal of Cardiac Implantable Electronic Device Leads,” Revista Portuguesa de Cardiologia [Portuguese Journal of Cardiology] 34(12): 739-744; Goyal, S. K., Ellis, C. R., Ball, S. K., Ahmad, R., Hoff, S., Whalen, S. P., and Rottman, J. 2014. “High-risk Lead Removal by Planned Sequential Transvenous Laser Extraction and Minimally Invasive Right Thoracotomy,” Journal of Cardiovascular Electrophysiology 25(6): 617-621; Paraskevaidis, S., Konstantinou, D., Vassilikos, V., Theofilogiannakos, E., Mantziari, L., and 3 others 2014. “Percutaneous Extraction of Transvenous Permanent Pacemaker/Defibrillator Leads,” Online, BioMed Research International 2014:949785; Tanawuttiwat, T., Gallego. D., and Carrillo, R, G. 2014. “Lead Extraction Experience with High Frequency Excimer Laser,” Pacing and Clinical Electrophysiology 37(9): 1120-1128; Okamura, H., Yasuda, S., Sato, S., Ogawa, K., Nakajima, I., and 8 others 2013. “Initial Experience Using Excimer Laser for the Extraction of Chronically Implanted Pacemaker and Implantable Cardioverter Defibrillator Leads in Japanese Patients,” Journal of Cardiology 62(3): 195-200; Zhou, X., Jiang, H., Ma, J., Bakhai, A., Li, J., and 7 others 2013. “Comparison of Standard and Modified Transvenous Techniques for Complex Pacemaker Lead Extractions in the Context of Cardiac Implantable Electronic Device-related Infections: A 10-year Experience,” Europace 15(11): 1629-1635; Madhavan, M., Swale, M., Gard, J. J., Hayes, D, L., and Asirvatham, S. J. 2012. “Contemporary Pacemaker and ICD Lead Management: Techniques for Lead Extraction,” Expert Review of Cardiovascular Therapy 10(7): 875-887; Maytin, M., Carrillo, R. G., Baltodano, P., Schaerf, R. H., Bongiorni, M. G., and 5 others 2012. “Multicenter Experience with Transvenous Lead Extraction of Active Fixation Coronary Sinus Leads,” Pacing and Clinical Electrophysiology 35(6): 641-647).

Furthermore, the assertion that adherent lead fragments disallow the use of magnetic resonance imaging is misconceived (see, for example, Han, D., Kang. S. H., Cho. Y., and Oh, I. Y. 2019. “Experiences of Magnetic Resonance Imaging Scanning in Patients with Pacemakers or Implantable Cardioverter-Defibrillators,” Korean Journal of Internal Medicine 34(1): 99-107; Maass, A. H., Hemels, M, E. W., and Allaart, C. P. 2018. “Magnetic Resonance Imaging in Patients with Cardiac Implantable Electronic Devices,” Netherlands Heart Journal 26(12): 584-590; Austin, C. O., Landolfo, K., Parikh, P. P., Patel, P. C., Venkatachalam, K, L., and Kusumoto, F. M. 2017. “Retained Cardiac Implantable Electronic Device Fragments are Not Associated with Magnetic Resonance Imaging Safety Issues. Morbidity, or Mortality after Orthotopic Heart Transplant,” American Heart Journal 190:46-53; Nazarian, S., Hansford, R., Rahsepar, A. A., Weltin, V., MeVeigh, D., and 9 others 2017. “Safety of Magnetic Resonance Imaging in Patients with Cardiac Devices,” New England Journal of Medicine 377(26): 2555-2564; Russo, R. J., Costa, H. S., Silva, P. D., Anderson, J, L., Arshad, A., and 18 others 2017. “Assessing the Risks Associated with MRI in Patients with a Pacemaker or Defibrillator,” New England Journal of Medicine 376(8): 755-764).

The procedure, described below, serves to add an intact if imperfect but reasonably competent second heart, preferably orthotopically in the chest but infrarenally in the abdomen, or in the pelvis if necessary, to that native without removing or surgically wounding either heart, both of which remain intact. The procedure is the same as in an orthotopic switch transplant, except that the native heart, because it can contribute ventricular function, allows the use of a less than ideal transplant heart, positioned beside the native heart in the chest when space allows or otherwise heterotopically, such as abdominally. If the native heart is so impaired that it cannot contribute any meaningful ventricular function, or absolute stroke volume, then two less than ideal hearts, either of which would have been discounted as suitable for transplantation on its own but which working together can be made to approximate the function of one dependable heart are placed.

The object in these procedures is to considerably expand the zone of hearts available for transplantation. In contrast to convention, using the switch technique, given a native heart nearing complete failure such that the native heart is considerably more impaired than either of two less than ideal donor hearts, either the native heart is first replaced with another less than ideal intact heart taken from a deceased donor on somatic support before a second less than ideal intact heart is added from a like donor, the major vessels of the two then joined, or two less than ideal intact replacement hearts from deceased donors on somatic support already taken from a first donor and placed in a second donor will have been joined in an earler procedure, the two then transplanted as paired into the recipient whose native heart is then removed.

Always true of allogeneic transplantation, it is important to obtain as good a match between donor and recipient as possible from the start. Sudden and metered switch organ transplantation differ in the transit time of the diversion valves until the graft organ has been completely switched from the circulatory system of the donor and into that of the recipient whose native organ has been bypassed. In metered compound vascular bypass, or sudden switched-organ transplantation, the extended time allows the shared circulation between recipient and donor of mutually chimerizing materials progressively increased over time until the process is considered complete.

The reciprocal cross-circulation increasingly cross-transfuses blood from throughout the bodies of donor and recipient as the process of transfer from the circulatory system of the donor into that of the recipient continues. The blood has therefore perfused and swept over the endothelia of all the glands and organs of both. In sudden compound vascular bypass, or sudden switched-organ transplantation, the procurement of chimerizing materials and monitoring of the results is periprocedural, which substantially excludes the interval during which the diversion chutes make the full traverse which is sudden.

While pre- and postprocedural treatment is not discounted, a primary object in metered compound vascular bypass, or sudden switched-organ transplantation is setting the duration of the transit interval of the chutes to allow as much chimerizing material to enter and circulate through the shared bloodstream as possible, that not possible administered periprocedurally. To accomplish a reasonable approximation requires expert evaluation of the likelihood that the implant prescription-program will slow down, reverse, or fully retract the chutes to truncate the process or will release a reversal agent which would increase the duration. The longer the transit time, the more is the blood of either represented in the shared circulation until a 50/50 mix is reached. Continued thereafter, the mix can continue to microchimerize both participants.

The significance of chimerization during shared circulation is that the process is continuously monitored by the implant sensors and the extracorporeal controller for both donor and recipient based upon the instant effect exerted by the shared bloodstream, with adjustments in the extent of chute traversal and appropriate reversal agents and/or other drugs released in the correct doses applied instantly when an adverse reaction is detected. The process is therefore safer than were blood draws manually exchanged between the participants on an intermittent basis. Once the valve-jackets have been placed and confirmed to function as required, the access incision can be closed as soon as the graft organ is removed from the donor and placed in the recipient.

Closure can be with the valves left in place indefinitely, and where the diseased organ was supported by diseased vessels, this is done to use the valve-jackets and lines as a prosthesis made entirely of synthetic materials and therefore not susceptible to whatever condition caused the deterioration of the native organ and its supply and drainage vessels. The duration of a metered switch transplant is limited on the donor side by the period over which the donor can be sustained in a competent condition. This time will continue to increase with added insight and working experience.

When general anesthesia is unavoidable, which circumstance switch transplantation should overcome, the duration of the procedure for the recipient, who is not on cardiopulmonary support or intubated, is determined by the time the patient can be kept anesthetized (see, for example, Graylee, G. P., Shaw, A. D., and Bartels, K. 2018. Hensley's Practical Approach to Cardiothoracic Anesthesia, Philadelphia, Pennsylvania: Lippincott, Williams and Wilkins/Wolters Kluwer; Gebhard Wagener (ed.) 2018. Liver Anesthesiology and Critical Care Medicine, New York, New York: Springer; Subramaniam, K. and Sakai, T. (eds.) 2017. Anesthesia and Perioperative Care for Organ Transplantation, New York, New York: Springer); Hadzic, A. 2017. Hadzic's Textbook of Regional Anesthesia and Acute Pain Management, New York, New York: McGraw-Hill Medical Publishing Division; Davis, P. J. and Cladis, F. P. (eds.) 2016. Smith's Anesthesia for Infants and Children, Amsterdam, North Holland, Netherlands: Elsevier; Kaplan, J. A., Augoustides, J, G. T., Reich, D, L., and Manecke, G, R. (eds.) 2016. Farag, E. and Mounir-Soliman, L. 2016. Brown's Adas of Regional Anesthesia, Amsterdam, North Holland, Netherlands: Elsevier; Kaplan's Cardiac Anesthesia: In Cardiac and Noncardiac Surgery, Amsterdam, North Holland, Netherlands: Elsevier; Sood, J. and Vohra, V. 2014. Anesthesia for Transplant Surgery, New Delhi, India: Jaypee Brothers Medical Publishers).

The avoidance of general anesthesia offering several advantages in any case, switch transplantation may allow the use of local and regional anesthesia without cardiopulmonary bypass or intubation in organ transplantation, and therewith, overcome the limitation in procedural duration imposed by the use of general anesthesia. The avoidance of general anesthesia in conventional organ transplantation is essentially limited to the kidneys (see, for example, Chen, W., Zhang, C., Wang, G., Li, Z., Wang, H., and Liu, H. 2017. “The Feasibility and Safety of Thoracoscopic Surgery under Epidural and/or Local Anesthesia for Spontaneous Pneumothorax: a Meta-Analysis,” Wideochirurgia i Inne Techniki Maloinwazyjne [Videosurgery and Other Miniinvasive Techniques] 12(3): 216-224; Paliwal, B., Kamal, M., Chauhan, D. S., and Purohit, A. 2016. “Off-pump Awake Coronary Artery Bypass Grafting under High Thoracic Epidural Anesthesia,” Journal of Anaesthesiology, Clinical Pharmacology 32(2): 261-262; El-Boghdadly, K., Madidpour, C., and Chin, K. J. 2016. “Thoracic Paravertebral Blocks in Abdominal Surgery—A Systematic Review of Randomized Controlled Trials,” British Journal of Anaesthesia 117(3): 297-308; Srivastava, V. K., Agrawal, S., Das, P. K., and Ahmed, M. 2014. “Low Dose Spinal with Epidural Volume Extension for Renal Transplantation in a Patient with Uremic Cardiomyopathy,” Indian Journal of Anaesthesia 58(1): 93-94; Ricaurte, L., Vargas, J., Lozano, E., and Diaz, L. 2013. “Anesthesia and Kidney Transplantation,” Transplantation Proceedings 45(4): 1386-1391; Watanabe, G., Tomita, S., Yamaguchi, S., and Yashiki, N. 2011. “Awake Coronary Artery Bypass Grafting under Thoracic Epidural Anesthesia: Great Impact on Off-pump Coronary Revascularization and Fast-track Recovery,” European Journal of Cardiothoracic Surgery 40(4): 788-793; Nicholls, A. J., Tucker, V., and Gibbs, P. 2010. “Awake Renal Transplantation; A Realistic Alternative to General Anesthesia,” Transplantation Proceedings 42(5): 1677-1678; Pompeo, E. and Mineo, T, C. 2008. “Awake Operative Videothoracoscopic Pulmonary Resections,” Thoracic Surgery Clinics 18(3): 311-320).

Sudden and metered compound vascular bypass, or sudden switched-organ transplantation transfer the burden of more detailed expertise from the surgeon to the immunologist, and unlike surgical expertise, immunological expertise can be conveyed from one distant expert to numerous sites. Other transplantation procedures—in adults with congestive heart failure, or to replace a kidney or segment of gut, for example, are less complex or delicate, the details readily inferred on the basis of a review of the more complex operation. However, the competent completion of the remedial measure is paramount, a simpler procedure done well is preferable to a complex one done poorly, and the warding off of hyperacute rejection, thrombus, infection, and malignancy is already advanced and becoming more so.

This may encourage the use of transplantation where it might have been avoided with a repairable heart discarded, denying it to a patient whose condition is more exigent, and even if the first patient cannot be brought to an optimal status, temporary repair will usually sustain him, albeit with progressive organ degradation, until a replacement heart can be found while a needier patient can be saved. However, any patient in need of a replacement organ must be listed with a central registriy which weighs these considerations and dispenses each organ to the best recipient. Palliative procedures are then applied only to bridge over the interval until a replacement heart or other organ can be found and chimereizing measures applied to both donor if on life support and recipient.

The highly developed immune system in adult patients makes automatic prosthetic disorder response system-targeted donor-recipient cross-chimerizing substances among multiple organs in the donor and recipient, either of whom may be keyed to one or plural partners, more complicated than it is early in the treatment of fetuses and neonates. In such a system, the use of ductus side-entry jackets allows the infusion of fluid chimerizing agents into any ductus, nonjacketing connectors with a hollow needle or hypotube, for example, allow the targeted injection of fluid agents into parenchymas, and the latter can apply adjunct therapy through an electrode or laser, for example, alongside the injection device or separate from it on another such connector.

Factors that must be considered are transplant to overall tissue mass ratio of the recipient, the functional diversity of the tissue, the diversity and number of enzymes and hormones involved, whether, contingent upon the condition of the recipient as well as immune interactions among the organs transplanted, to effect transplantation at the same time or sequentially, and so on. Thus, both due to the functional diversity and the mass of tissue as a proportion of all tissue in the body, to transplant the digestive structures immediately associated with it along with the liver should effect a more potent and diverse chimerization to promote tolerance.

Such evidence as is available indicates that the prospects for success rise with increased mass and functional diversity of transplanted material, so that a liver and multiple organ transplants tend to induce greater chimerization, hence, accommodation, than does transplantation of a single organ such as a kidney with less tissue mass and extensive physiology. This combined with improved excretory function improve the odds for success in a patient with cardiorenal impairment when same donor kidney accompanies heart transplantation (see, for example, Savla, J., Lin, K. Y., Pradhan, M., Ruebner, R, L., Rogers, R. S., and 6 others 2015, Op cit.; Kilic, A., Grimm, J. C., Whitman, G. J., Shah, A. S., Mandal, K., Conte, J. V., and Sciortino, C. M. 2015. “The Survival Benefit of Simultaneous Heart-kidney Transplantation Extends Beyond Dialysis-dependent Patients,” Annals of Thoracic Surgery 99(4): 1321-1327; Karamlou, T., Welke, K. F., McMullan, D. M., Cohen, G. A., Gelow, J., and 4 others 2014, Op cit., section 2a(3), entitled Remotely Procured Graft Organ Preservation; Gill, J., Shah, T., Hristea, I., Chavalitdhamrong, D., Anastasi, B., Takemoto, S. K., and Bunnapradist, S. 2009. “Outcomes of Simultaneous Heart-kidney Transplant in the US: A Retrospective Analysis Using OPTN/UNOS [Organ Procurement and Transplantation Network/United Network for Organ Sharing] Data,” American Journal of Transplantation 9(4): 844-852; Pinderski, L. J., Kirklin, J. K., McGiffin, D., Brown, R., Naftel, D. C., and 11 others 2005, “Multi-organ Transplantation: Is There a Protective Effect Against Acute and Chronic Rejection?,” Journal of Heart and Lung Transplantation (New York, New York) 24(11): 1828-1833).

For example, the 10 year graft survival rate for transplantation of the intestine alone is 26 percent, but increases to 40 percent when combined with transplantation of the liver with or without the pancreas, and while including the liver improves outcomes, it is more susceptible to infection, which, however, is more amenable to resolution than is rejection (Wu, G., Cruz, R. J. Jr. 2018. “Liver-inclusive Intestinal Transplantation Results in Decreased Alloimmune-mediated Rejection but Increased Infection,” Gastroenterology Report (Oxford, England) 2018 6(1): 29-37; Nagai, S., Safwan, M., Collins, K., Schilke, R. E., Rizzari, M., and 5 others 2018, Op cit.; Hussain, S. M. and Sureshkumar, K. K. 2018, Op cit.; Bharadwaj, S., Tandon, P., Gohel, T. D., Brown, J., Steiger, E., and 3 others 2017, Op cit.; Huard, G., Schiano, T., Moon, J., and Iyer, K. 2017. “Choice of Allograft in Patients Requiring Intestinal Transplantation: A Critical Review,” Canadian Journal of Gastroenterology and Hepatology 2017:1069726; Taner, T., Heimbach, J. K., Rosen, C. B., Nyberg, S, L., Park, W. D., and Stegall, M, D. 2016. “Decreased Chronic Cellular and Antibody-mediated Injury in the Kidney following Simultaneous Liver-kidney Transplantation,” Kidney International 89(4): 909-917; Ramisch, D., Rumbo, C., Echevarria, C., Moulin, L., Niveyro, S., and 10 others 2016. “Long-term Outcomes of Intestinal and Multivisceral Transplantation at a Single Center in Argentina,” Transplantation Proceedings 48(2): 457-462).

Less discrepant figures for shorter term graft survival are likely attributable to the greater complexity and the need for greater accumulated experience in the medical management of the more encompassing physiology involved in multiple organ transplantation and the basing of the choice of organs to be transplanted more if not entirely upon symptoms more, or rather than, chimerizing strategy. Currently the choice of isolated jejunoileal, jejunoileal-liver, and multivisceral transplantation that includes stomach, duodenum, with or without the liver and pancreas, is based upon symptoms more or rather than chimerizing strategy (Huard, G., Schiano, T., Moon, J., and Iyer, K. 2017, Op cit.).

In addition to the bloodline switch scheme for lengthening or shortening a mainline for passing blood or urine shown in FIGS. 32A thru 32G whereby a long is switched to another shorter line of the reverse, mainlines for passing blood or urine can be made with the alternating flats and vaults configuration expandable fabric described below in section 2b(2)(f), entitled Expandable Structure to Accommodate Growth and Self-adjust in Size in Vascular Segment Prostheses, Lines, and Confluence Conduits Placed in Children. Spontaneously enlarging in response to growth, this has the advantage of eliminating the need for reentry.

Yet another way to lengthen a mainline, considered an emergency expedient when an alternative means is unavailable as might arise in an exigent sudden switch transplantation, consists of spanning, that is, bridging or bypassing over a prospective transection of the line and connecting the ends of the bridge to the line to either side of the prospective transection with a flow diversion valve at the entry end and a simple flow diversion jacket at the exit end of the bridge before actually transecting the line. The reverse action of shortening a line having been lengthened thus consists of bonding the cut ends of the line and closing the entry valve so that flow is straight through the line.

The bridge past the entry valve is then snipped off. As to the means for bonding the free ends previously bridged over, these can be joined with several turns of a suitable implantable tape. An implantable tape must not be susceptible to enzymatic or hydrophilic breakdown, must not provoke a more problematic adverse foreign body immune reaction, and must persist as strongly adherent to the substrate whether tissue or a synthetic. Thread seal tape having been determined to provoke little if any adverse reaction when implanted, the single-sided addition of an adhesive coating similarly unproblematic would be appropriate to create the joint indicated (Veterbo, F. Batalha, P. K., and Bacchi, C, E. 2006. “Thread Seal Tape (Polytetrafluoroethylene) Implanted in the Subcutaneous Tissue of Rats,” Anesthetic Plastic Surgery 30(1): 77-80).

That adhesion can be wet for application to living tissue rather than commercial or tissue engineered tubing of stretchahle rubbery character and a surface essential for the formation of the covalent bonding essential for adhesion of the tape to tissue as such is uninvolved, and the ability to adhere in water attests to a lack of susceptibility to enzymatic or hydrophilic breakdown (see, for example, Zou, C. Y., Lei, X. X., Hu, J. J., Jiang, Y, L., Li, Q. J., and 4 others 2022. “Multi-crosslinking Hydrogels with Robust Bioadhesion and Pro-coagulant Activity for First-aid Hemostasis and Infected Wound Healing,” Online, Bioactive Materials 16:388-402; Xue, B., Gu, J., Li, L., Yu, W., Yin, S., and 4 others 2021. “Hydrogel Tapes for Fault-tolerant Strong Wet Adhesion,” Online, Nature Communications (London, England) 12:7156; Fu, Y., Ren, P., Wang, F., Liang, M., Hu, W. and 3 others 2020. “Mussel-inspired Hybrid Network Hydrogel for Continuous Adhesion in Water,” Journal of Materials Chemistry B. Biology and Medicine 8(10): 2148-2154).

If the need therefor had been anticipated, then connection with an end-inserted connector such as that shown in FIG. 31C (simplified in omitting accessory channels), into tubing that had been chosen beforehand for retention of the hooks would be satisfactory. Ordinarily meant to be inserted into the cut end of a ductus such as a vessel so that its retentive hooks engage to prevent extraction, the tubing employed should allow the hooks to be forcibly engaged into the internal surface surrounding the lumen without being so soft as to risk kinking or collapse.

As long as the diversion chutes in the subsidiary diversion jackets remain fully extended, blood is diverted from just short of the free end of the downstream jacket into the length-contributing diversionary bypass to just short of the free end of the upstream jacket to afford the extra length needed for the surgical team to function without the hindrance due to a lack of sufficient space in which to maneuver. Once the transplant organ is placed in the recipient, the subsidiary diversion jacket chutes are fully retracted to allow blood to flow directly through the mainline with the extension removed.

The subsidiary jackets and bypass are then removed and the openings for the jackets in the mainline sealed shut. Otherwise, the jackets can be left in place so that the accessory channels will remain available to target medication into the mainlines. In most instances, the bypass is removed and the jacket side connectors, but not the accessory channels, sealed shut. Other such arrangements are possible, but diversion chutes are preferred as gently redirecting blood into the bypass, minimizing hemolysis, affording flexibility in positioning along the mainlines, if appropriate, allowing flow through a larger than native lumen to be adjusted, and furnishing accessory channels for continued targeted drug therapy from a port at the body surface after the recipient has been closed.

As described below, in fetuses, neonates, and small infants, to accommodate growth over as long a period as possible without the need for reentry, the mainlines consist of a strong film used to line plastic low force helical tension/extension springs that gently adjust to the shortest distance between the spring ends. The prosthetic blood-conducting tubing, or mainlines, provide somatotropin, or growth hormone, no less than native arteries. In a sudden or metered switch organ procedure, the jackets on the donor are oriented for incurrent arterial flow and excurrent venous flow, while those on the recipient are oriented for excurrent arterial flow and incurrent venous flow, those on the donor therefore inverted.

In excurrent use, adjustable side-entry diversion jackets allow controlling the cross-sectional area at the diversion to continuously vary the apportionment of flow between the native route and the shunt or bypass by rotating a knob in a port at the body surface, the port itself a modified nonjacketing side-entry connector as described in U.S. Pat. No. 11,013,858. In incurrent use, side-entry diversion jackets allow an infusate to be adjusted in delivery rate in the same manner. In open surgery, such as a carotid endarterectomy, one diversion jacket on the common carotid bypasses to side-entry jackets on the internal and external carotids.

The diversion jacket or valve chute can be manually shifted from the extended position, that is, fully extended into the native lumen or the substrate vessel encircled, whereby the native lumen is fully closed off, or obturated, allowing bypass outflow or diversion from beneath it to the internal and external carotids to the retracted or diversion chute bypassed position by the operator, whereupon normal flow is restored. The upper surface of the chute is configured to least disrupt laminar flow.

If following injury or resection as malignant, for example, the condition of the carotid does not permit removal of the device using vascular servovalves of the type shown in shown in FIG. 25A or 25B and described below in section 2b(2)(g), entitled Ischemia and Hypoperfusion-free Carotid Endarterectomy Bypass and Prosthesis, the device is left in place as a permanent prosthesis. Following an endarterectomy or repair of an aneurysm, whether used for the procedure or to stay as a prosthesis, the three jackets remain in place so that the accessory lines of each can be used to directly target medication to their respective supply territories.

A diversion or chute valve is unlike a choke valve without diversion; however, in diverting a portion of the flow, the pressure in the diverted portion increases and extends upstream in proportion to the extent that the angle of incidence of the oncoming blood and surface of the chute are perpendicular, the volume of blood diverted proportional to the degree of extension of the chute into the lumen, the portion of flow beyond the chute continuing through the native lumen. Such a valve is used to divert a portion of the flow to a neighboring artery through a conotruncal outflow vessel in order to ameliorate a discordance between the two to the extent that correction can be accomplished outside the heart. In most instances, it is best to set the level and rotational angle of the chute for the least shear stress.

2b(1)(a). Sudden Compound Vascular Bypass, or Sudden Switched-Organ Transplantation

Sudden compound vascular bypass, or sudden switched-organ transplantation, achieves zero ischemic time, hence, zero reperfusion injury, in organ transplantation by connecting the corresponding vessels of recipient and donor organs with coiled elastic lines. The lines, free of luminal projections or sharp turns, are connected to the recipient and donor vessels, or if the donor is unavailable, then to the perfusion machine system lines at either end by means of intravascular diversion valve-jackets, which allow flow through the substrate vessel to be redirected for continued flow through the other circulatory system or lines of the organ transport support system.

The method is suitable for prospective recipients with an immature or impaired immune system not likely to react with intensity when suddenly confronted with an alien organ. In adults, its use should be limited to patients in imminent danger of death. In both a sudden and a metered switch solid organ transplantation as described below in the section to follow, neither the native organ to be replaced nor the donor organ is ever left short of the full complement of blood; rather, the blood supply and drainage of each are transferred from the donor to the recipient.

In sudden switch compound vascular bypass transplantation, the continued sustainment of the deceased donor to allow the harvesting of additional organs is no less important than in metered bypass transplantation. For this reason, arterial and venous circulation is sustained continuously in the donor following the procedure as it is in the recipient, so that following harvesting of the heart, circulation in the donor is sustained by mechanical means.

In a sudden compound vascular bypass, or sudden switched-organ transplant, the transfer is abrupt so that the donor organ will have a negligible proportion of recipient blood, so that preprocedural immune tolerance induction is important. In contrast, in a metered compound vascular bypass, or sudden switched-organ transplant, the exchange of cross-delivery, that is, the reciprocal cross-circulation, of recipient circulation to the donor organ is carried out gradually, so that the composition of the blood in either will include the blood of the other in proportion to the interval over which their blood is allowed to mix.

Precisely because the introduction of an alien organ into the donor in a sudden switch is abrupt, it is important that the donor well match the recipient, and that the immune response be as dulled as possible without rendering the graft or the recipient excessively immunocompromised. Accordingly, the periprocedural application of immunosuppressants must be expert. While rarely would the patient not have been admitted well before a terminal event, in such an eventuality, immune tolerance-inducing substances and immunosuppressant therapy should be initiated upon arrival and the patient closely monitored for an adverse reaction.

Because the solenoid diversion chutes can be reversed by a current surge high enough in amplitude to pull the plungers out of the detents, and when fully retracted, all flow passes through the native organ, the transplant can be effectively removed from the recipient circulatory system in an instant. This resolution can be incorporated into the prescription-program or the same action achieved by manual override. Such a circumstance argues in favor of metered compound vascular bypass, or sudden switched-organ transplantation whereby the transfer between donor and recipient circulatory systems is carried out no more quickly than the implanted rejection analyte sensors indicate the recipient is tolerating. If appropriate, the diversion chutes can be fully retracted for an interval and thereafter the process continued or ended, such action, repeated as often as necessary, programmed into the control, or prescription-program.

Two hearts are parts of two circulatory systems throughout. With neither method for single or orthotopic heart transplantation is the graft organ left hypovolemic as to necessitate a transfusion. In a solid organ transplant, such as an off-pump circulation disruption-minimized heart transplant, where a diversion jacket must be placed on each vessel of both the donor and recipient organs, then switched from the chute-extended to the retracted position simultaneously to redirect recipient circulation from the innate to the transplant heart, switching of the diversion chutes is accomplished with a switch that actuates the solenoid, damped to prevent abrupt, jerking motion, on each vessel, the jackets functioning as vascular electrovalves.

The utility of side-entry jackets is not, however, limited to return jackets for delivery through a bypass from a side-entry diversion jacket: if anastomosed rather than allowed flow to proceed through the mainline, the suture lines and tissues in conventional orthotopic and heterotopic transplants-potentially sources of ectopic electrical discharge—are beneficially served through the direct targeting from a small subdermalliy placed port of adverse tissue reaction immunosuppressants, anti-inflammatoires, and should it become necessary, antimicrobials.

In a compound vascular bypass or switched-organ heterotopic double heart transplant, addressed below in section 2b1(b)(b5), the donor heart arterial and venous bloodlines are finally disconnected, so that the donor heart is removed with only the blood within it. Cut off from the balance of blood in the circulatory system of the donor, the two hearts must now function with the volume of blood in not two but rather one circulatory system. Rather than to allow both hearts to undergo sustained hypovolemia, the recipient is transfused with sufficient blood to support both hearts. While venous retum must be still divided between the two hearts, delivery into each is at the someone higher pressure resulting from the increase in blood volume, and this increase in pressure is not communicated to either the pulmonary or systemic circulation. Additional means consisting of adjusting the diversion servovalves are described in section 2b(1)(b)(b5), entitled Heterotopic Double Heart Metered Compound Vascular Bypass, or Switched-organ Transplantation [and so on].

More suitable for sudden compound vascular bypass, or sudden switched-organ transplantation are the very young with an immature immune system, adults in imminent danger of death such as those sustained with the aid of an artificial heart or ventricular assist device, and those for whom collateral cellular materials from the donor are no longer usable for the purpose of immune tolerance induction as would permit the application of metered compound vascular bypass, or sudden switched-organ transplantation. Due to the abruptness of confrontation between immunologically distinct tissue.

As indicated, sudden compound vascular bypass, or sudden switched-organ transplantation is intended to be reserved for emergency replacement of an end-stage organ where death is imminent. It uses highly damped nonsparking solenoid-driven diversion valves that either fully extended or fully retracted, are bistable. Solenoid-driven valves can be reversed but not adjusted to intervening degrees of extension, making these inflexible compared to servovalves, which can be adjusted at any rate to any succession of extensions.

In any organ transplantation using servovalves, the microcontroller, based upon inputs from sensors integral in the valves, is able to coordinate, or tune, the complement of individual valves so that organ in- and outflow is continuously optimized. When transplantation is sudden using solenoid valves, this continuing capability is precluded from the outset. During transplantation, before the patient is closed, this allows use of the microcontroller operating under the prescription-program to determine the optimal combination of valve settings. When the implanted automatic disorder response system is provided with sensors and programmed to monitor and treat comorbid disease, the servovalves and drug releasing reservoir outlet pumps or drip stopcocks can be continuously adjusted to accomplish optimal homeostasis for the patient.

The desirability of preserving the entire body and not just the graft organ argues for the creation of centralized facilities for this express purpose with immediate access by air. The deceased donor should have been maintained past death on life support at the same center as the recipient. Generally, it is better to transport the prospective recipient to the center maintaining the deceased donor than the reverse. If at a remote location where transport of the recipient or the deceased intact is problematic, the organ can be directly bypass, or sudden switched-organ transferred before removal from the deceased donor while remaining on life support to an organ transport support system that provides normothermic or mildly chilled pulsatile perfusion (references cited above in section 2. Background of the Invention a. General (3). Remote Graft Organ Preservation. The process of reciprocal cross transfusion is accelerated when blood of the recipient is used during somatic support.

Heart failure in a child usually differs in kind from that in an adult, in the former it is usually due to one or more congenital defects, while in the latter it is usually due to end stage heart failure. If and only if the condition is determined to be terminal, children below the age of 18 years with end-stage heart disease are heart transplanted, a mechanical assist device, sometimes referred to as a “heart pump” used as a bridge until a replacement heart is obtained (Metra, M., Dinatolo, E., and Dasseni, N. 2019. “The New Heart Failure Association Definition of Advanced Heart Failure,” Cardiac Failue Review 5(1): 5-8; Taylor, C. J., Ordonez-Mena, J. M., Roalfe, A. K., Lay-Flurrie, S., Jones, N. R., Marshall, T., and Hobbs, F. D, R. 2019. “Trends in Survival after a Diagnosis of Heart Failure in the United Kingdom 2000-2017: Population Based Cohort Study,” Online, British Medical Journal (Clinical Research) 364:1223; Taylor, C. J., Ryan, R., Nichols, L., Gale, N., Hobbs, F. R., and Marshall, T. 2017. “Survival following a Diagnosis of Heart Failure in Primary Care,” Family Practice 34(2): 161-168; Combes, A. 2017. “Mechanical Circulatory Support for End-stage Heart Failure,” Metabolism 69S: S30-S35; Jones, N. R., Hobbs, F. R., and Taylor, C. J. 2017. “Prognosis following a Diagnosis of Heart Failure and the Role of Primary Care: A Review of the Literature,” British Journal of General Practitioners Open 1(3): bigpopen17X101013; Stokes, M. B., Bergin, P., and MeGiffin, D. 2016. “Role of Long-term Mechanical Circulatory Support in Patients with Advanced Heart Failure,” Internal Medicine Journal 46(5): 530-540; Pocock, S., Ariti, C. A., McMurray, J. J., Maggioni, A., Kober, L., and 6 others 2013. “Predicting Survival in Heart Failure: A Risk Score Based on 39, 372 Patients from 30 Studies,” European Heart Journal 34(19): 1404-1413; Korewicki, J., Leszek, P., Zielinski, T., Rywik, T., Piotrowski, W., and 4 others 2012. “Severe Chronic Heart Failure in Patients Considered for Heart Transplantation in Poland,” Cardiology Journal 19(1): 36-44; Korewicki, J. 2009. “Cardiac Transplantation is Still the Method of Choice in the Treatment of Patients with Severe Heart Failure,” Cardiology Journal 16(6): 493-499) as would an adult under nonexigent circumstances, using the metered switch technique.

The placement of an intact second heart in the lower abdominal cavity, infrarenally in the abdomen, or within the pelvis, as addressed below in section 2b(1)(b)(b5), entitled Heterotopic Double Heart Metered Compound Vascular Bypass, or Switched-organ Transplantation [and so on], may avert the need for an interim assist device in parts of the world where none is available or affordable. Cardiomyopathy and/or congenital defects causes of renal malfunction, the diagnosis, diagnostics-based tracking, and risk stratification of heart failure in a child or an adult awaiting a heart transplant can be obtained from the blood pressure and through examination of the blood, urine, and saliva (see, for example, Ahmed, F. Z., Sammut-Powell, C., Kwok, C. S. Tay, T., Motwani, M., Martin, G. P., and Taylor, J. K. 2022. “Remote Monitoring Data from Cardiac Implantable Electronic Devices Predics All-cause Mortality,” Online, Europace 24(2): 245-255; Fountoulaki, K., Ventoulis, Drokou, A., Georgarakou, K., Parissis, J., and Polyzogopoulou, E. 2022. “Emergency Department Risk Assessment and Disposition of Acute Heart Failure Patients: Existing Evidence and Ongoing Challenges,” Online, Heart Failure Review 20:1-3; Sammut-Powell, C., Taylor, J. K., Motwani, M., Leonard, C. M., Martin, G. P., and Ahmed, F. Z. 2022. “Remotely Monitored Cardiac Implantable Electronic Device Data Predict All-cause and Cardiovascular Unplanned Hospitalization,” Journal of the American Heart Association (Oxford, England) 11(16): e024526; Zile, M. R., Koehler, J., Sarkar, S., and Butler, J. 2020. “Prediction of Worsening Heart Failure Event and All-cause Mortality Using an Individualized Risk Stratification Strategy,” Online, ECS [European Society of Cardiologists] Heart Failure (Oxford, England) 7(6): 4277-4289; Collins, S. P. am Pang. P. S. 2019. “ACUTE Heart Failure Risk Stratification,” Online, Circulation 139(9): 1157-1161; Forte, M., Madonna, M., Schiavon, S., Valenti, V., Versaci, F., and 3 others 2019; Op cit. section 2a; Gangnus, T. and Burckhardt, B, B. 2019. “Potential and Limitations of Atrial Natriuretic Peptide as Biomarker in Pediatric Heart Failure-A Comparative Review,” Online, Frontiers in Pediatrics 6:420; Rubattu, S. and Volpe, M. 2019. “Natriuretic Peptides in the Cardiovascular System: Multifaceted Roles in Physiology, Pathology, and Therapeutics,” Online, MDPI [Multidisciplinary Digital Publishing Institute] International Journal of Molecular Sciences (Basel, Switzerland) 20(16). pii: E3991; Bie, P. 2018. “Natriuretic Peptides and Normal Body Fluid Regulation,” Comprehensive Physiology 8(3): 1211-1249; Das, B. B. 2018. “Current State of Pediatric Heart Failure,” Children (Basel, Switzerland) 5(7). pii: E88; Joharimoghadam, A., Tajdini, M., and Bozorgi, A. 2017. “Salivary B-type Natriuretic Peptide: A New Method for Heart Failure Diagnosis and Follow-up,” Kardiologia Polska 75(1): 71-77; Hinton, R, B. and Ware, S. M. 2017, Op cit. above under 1. Background of the Invention. a. General; Jayaprasad, N. 2016. “Heart Failure in Children” Heart Views 17(3): 92-99; Bajcetic. M., Uzelac. T. V., and Jovanovic, I. 2014. “Heart Failure Pharmacotherapy: Differences between Adult and Paediatric Patients,” Current Medicinal Chemistry 21(27): 3108-3120; Zielinski, T., Browarek, A., Zembala, M., Sadowski, J., Zakliczynski, M., and 4 others with 24 collaborators 2009. “Risk Stratification of Patients with Severe Heart Failure Awaiting Heart Transplantation-Prospective National Registry POLKARD HF [Polish national study of heart failure],” Transplantation Proceedings 41(8): 3161-3165; Gauquelin, G., Maillet. A., Allevard, A. M., Vorobiev, D., Grigoriev, A. I., and Gharib, C. 1992. Presence of Atrial Natriuretic Factor and Cyclic Guanosine Monophosphate in Saliva. Comparison of Plasma and Salivary Concentrations during a Head-down Tilt,” European Journal of Applied Physiology and Occupational Physiology 65(1): 25-29).

In a sudden compound vascular bypass, or sudden switched-organ transplantation, the circulation of the recipient through his own diseased organ is suddenly, and in a metered compound vascular bypass, or sudden switched-organ transplantation gradually, switched to and through the corresponding healthy organ of the donor. The deceased donor is somatically supported by hematological, immunological, and cardiological intensivists. The sudden and metered switch techniques apply to prenates, neonates, infants, older children, and adults regardless of the organ to be transplanted, and includes the heart. Ductus side-entry diversion jackets are used as vascular valves to redirect circulation or perfusion of a solid organ to be transplanted from the recipient's diseased to the donor's healthy organ by switching between the counterpart supply and drainage vessels of the two.

A donor cerebrally dead with intact brain stem (brainstem) and preservation of autonomic function with the possibility of regaining consciousness is considered to remain alive, and is required by law to be supported with the expectation of recovery. While the diversion chute in each valve remains retracted, flow through its substrate vessel is unaffected. When the diversion valve chutes are advanced each into its respective lumen, flow through the recipient organ is diverted to pass through the donor organ. This compound vessel bypass from the innate recipient organ to and through the donor organ serves to switch the donor organ from the donor circulatory or support system to the circulatory system of the recipient with no interruption in perfusion.

While to harvest the organs of a patient in this condition is unlawful, support thus also maintains the usable organs in a better condition should death ensue. The patient is then, kept in condition for either recovery or transplant with life support by a ventilator (“respirator”) and/or extracorporeal membrane oxygenator, warmth, hormones and enzymes as necessary, and parenteral fluids and nutrition (see, for example, Shetty, V, L., Mali, S. S., Shetty, S. V., and Shinde, P, D. 2017, Op cit.; Kumar 2016. Op cit.). Since ideally there is no ischemic or chilled storage time, the conventional requirement to take the heart first, the liver second, and the kidneys third does not apply. Conventionally, once a prospective organ donor has expired, the life support system is shut off, destroying and precluding donor cells from use to achieve preprocedural immune tolerance induction, organs for transplantation only then removed, after perfusion has been terminated.

This introduces a major complication at the outset, since once reinstated, perfusion can further and significantly impair the condition of the graft organ. Reperfusion injury primarily affects the endothelium, and as cited earlier in this section, the endothelium, a primary source of the rejection response; the damage is both physiological and immunological, affecting both sources of transplant failure, and provoking an injurious inflammatory response. Once ‘brain dead,’ without the aid of life support, irregular pulsation may continue pending cardiac arrest within a week (Kinoshita, Y., Kamohara, H., Kotera, A., Sagishima, K., Tashiro, T., and Niimori, D. 2014, Op cit. above under 1. Background of the Invention. a. General).

The immune response results in degradation, vasculopathy, of the endothelia of both host and graft, which it would do were less vascular allogeneic tissue sutured together, and with a graft inclusive of relatively large caliber vessels, reperfusion and shear stress combine with the inflammatory response to exact greater injury (see, for example, Aljabri, A., Vijayan, V., Stankov, M., Nikolin. C., Figueiredo, C., and 4 others 2019. “HLA Class II Antibodies Induce Necrotic Cell Death in Human Endothelial Cells via a Lysosomal Membrane Permeabilization-mediated Pathway,” Cell Death and Disease 10(3): 235; Cross, A. R., Glotz, D., and Mooney, N. 2018. Op cit., above in section 2a, entitled Background of the Invention. General; Penack, O. and Luft T. 2018. “Editorial: Endothelial Dysfunction during Inflammation and Alloimmunity,” Online, Frontiers in Immunology 9:2886; Morsing, K. S. H., Peters, A, L., van Buul. J. D., and Vlaar, A. P. J. 2018. “The Role of Endothelium in the Onset of Antibody-mediated TRALI [Transfusion Related Acute Lung Injury],” Blood Reviews 32(1): 1-7; Jin, Y. P., Valenzuela, N. M., Zhang, X., Rozengurt, E., and Reed, E. F. 2018. “HLA Class II-triggered Signaling Cascades Cause Endothelial Cell Proliferation and Migration: Relevance to Antibody-mediated Transplant Rejection,” Journal of Immunology 200(7): 2372-2390; Guo, X., Hu. J., Luo, W., Luo, Q., Guo, J., and 3 others 2017. “Analysis of Sera of Recipients with Allograft Rejection Indicates that Keratin 1 is the Target of Anti-endothelial Antibodies,” Journal of Immunology Research 2017:8679841; Wang. S., Zhang, C., Wang, J., Yang, C., Xu, M., and 3 others 2017. “Endothelial Cells in Antibody-mediated Rejection of Kidney Transplantation: Pathogenesis Mechanisms and Therapeutic Implications,” Journal of Immunology Research 2017:8746303; Tietjen, G. T., Hosgood, S. A., DiRito, J., Cui, J., Deep. D., and 12 others 2017. “Nanoparticle Targeting to the Endothelium during Normothermic Machine Perfusion of Human Kidneys,” Science Translational Medicine 9(418). pii: eaam6764; Boteon, Y, L., Laing. R., Mergental, H., Reynolds, G. M., Mirza, D. F., Afford, S. C., and Bhogal, R, H. 2017. “Mechanisms of Autophagy Activation in Endothelial Cell and Their Targeting during Normothermic Machine Liver Perfusion,” World Journal of Gastroenterology 23(48): 8443-8451; Westall, G. P., Snell, G. I., Loskot, M., Levvey, B., O'Hehir, R., Hedger, M. P., and de Kretser, D. M. 2017. “Activin Biology after Lung Transplantation,” Transplantation Direct 3(6): e159; Sanchez-Zapardiel, E., Mancebo, E., Diaz-Ordonez, M., de Jorge-Huerta, L., Ruiz-Martinez, L., and 7 others 2016. “Isolated De Novo Antiendothelial Cell Antibodies and Kidney Transplant Rejection,” American Journal of Kidney Diseases 68(6):933-943; de Kretser, D. M., Bensley, J. G., Phillips, D. J., Levvey, B. J., Snell, G. I., and 3 others 2016. “Substantial Increases Occur in Serum Activins and Follistatin during Lung Transplantation,” Online, PLoS [Public Library of Science] One 11(1): e0140948; Dragun, D., Catar. R., and Philippe, A. 2016. “Non-HLA Antibodies against Endothelial Targets Bridging Allo- and Autoimmunity,” Kidney International 90(2): 280-288; Rajalingam, R. 2016. “The Impact of HLA Class I-specific Killer Cell Immunoglobulin-like Receptors on Antibody-dependent Natural Killer Cell-mediated Cytotoxicity and Organ Allograft Rejection,” Online, Frontiers in Immunology (Lausanne, Switzerland) 7:585; Zilian, E., Saragih, H., Vijayan, V., Hiller, O., Figueiredo. C., and 6 others 2015. “Heme Oxygenase-1 Inhibits HLA Class I Antibody-dependent Endothelial Cell Activation,” Online, PLoS [Public Library of Science] One 10(12): e0145306; Jackson, A. M., Sigdel, T. K., Delville, M., Hsieh, S. C., Dai, H., and 3 others 2015. “Endothelial Cell Antibodies Associated with Novel Targets and Increased Rejection,” Journal of the American Society of Nephrology 26(5): 1161-1171; Snell, J. N., Westall, G. P., and Snell, G. I. 2015. “The Potential Role of Activin and Follistatin in Lung Transplant Dysfunction,” Expert Review of Respiratory Medicine 9(6): 697-701; Thaunat. O. 2015. “Soothing Touch of CD31 Protects Endothelium during Cellular Immune Responses,” Online, Proceedings of the National Academy of Sciences of the United States of America 112(43): 13133-13134; Piotti, G., Palmisano, A., Maggiore, U., and Buzio, C. 2014. “Vascular Endothelium as a Target of Immune Response in Renal Transplant Rejection,” Online, Frontiers in Immunology (Lausanne, Switzerland) 5:505; Hedger, M. P. and de Kretser, D. M. 2013. “The Activins and Their Binding Protein, Follistatin-Diagnostic and Therapeutic Targets in Inflammatory Disease and Fibrosis,” Cytokine and Growth Factor Reviews 24(3): 285-295; Ziegler, M. E., Souda, P., Jin. Y. P., Whitelegge, J. P., and Reed, E, F. 2012. “Characterization of the Endothelial Cell Cytoskeleton following HLA Class I Ligation,” Online, PLoS [Public Library of Science] One 7(1): e29472; de Kretser, D. M., O' Hehir, R. E., Hardy, C, L., and Hedger, M. P. 2012. “The Roles of Activin A and Its Binding Protein, Follistatin, in Inflammation and Tissue Repair,” Molecular and Cellular Endocrinology 359(1-2): 101-106; Taflin, C., Charron, D., Glotz, D., and Mooney, N. 2011. “Immunological Function of the Endothelial Cell within the Setting of Organ Transplantation,” Immunology Letters 139(1-2): 1-6; Vos, I, H. and Briscoe, D. M. 2002. “Endothelial Injury: Cause and Effect of Alloimmune Inflammation,” Transplant Infectious Disease 4(3): 152-159; Wu, G. D., Jin, Y. S., Salazar, R., Dai. W. D., Barteneva, N., and 4 others 2002. “Vascular Endothelial Cell Apoptosis Induced by Anti-donor Non-MHC Antibodies: A Possible Injury Pathway Contributing to Chronic Allograft Rejection,” Journal of Heart and Lung Transplantation (New York, New York) 21(11): 1174-1187).

Once achieved, lumen endothelialized bioengineered vascular grafts for incorporation into artificial organs should allow a reduction in endothelial injury and clot (see, for example, Peng. G., Yao. D., Niu. Y., Liu, H., and Fan, Y. 2019. “Surface Modification of Multiple Bioactive Peptides to Improve Endothelialization of Vascular Grafts,” Macromolecular Bioscience e1800368; Kutikhin, A. G., Sinitsky, M. Y., Yuzhalin, A. E., and Velikanova, E, A. 2018. “Whole-transcriptome Sequencing: A Powerful Tool for Vascular Tissue Engineering and Endothelial Mechanobiology,” High-throughput 7(1). pii: E5; Gurjarpadhye, A. A., DeWitt, M. R., Xu. Y., Wang, G., Rylander, M. N., and Rylander, C, G. 2015. “Dynamic Assessment of the Endothelialization of Tissue-engineered Blood Vessels Using an Optical Coherence Tomography Catheter-based Fluorescence Imaging System,” Tissue Engineering. Part C. Methods 21(7): 758-766; Ren, X., Feng. Y., Guo, J., Wang, H., Li, Q., and 5 others 2015. “Surface Modification and Endothelialization of Biomaterials as Potential Scaffolds for Vascular Tissue Engineering Applications,” Chemical Society Reviews 44(15): 5680-5742; Whited, B. M., Hofmann, M. C., Lu, P., Xu. Y., Rylander, C. G., and 6 others 2013. “Dynamic, Nondestructive Imaging of a Bioengineered Vascular Graft Endothelium,” Online, PLoS [Public Library of Science] One 8(4): e61275).

This interruption in normothermic pulsatile perfusion, often lengthy and involving refrigeration in a static or flowing chilled perfusate, reflects the lack of means to allow continuous perfusion throughout organ harvesting and repositioning into the circulatory system of the recipient. Thus, interruption, much less the complete cessation of normothermic pulsatile perfusion with transection and dissection, predispose to post-transplantation complications and the risk of failure, not just on its own, but as detracting from the ability to achieve and sustain immunological compatibility.

Previously mentioned above under 1. Background of the Invention. a. General. (3). Remote Graft Organ Preservation, rather than seeking to eliminate ischemic time and reperfusion injury, preliminary measures such as hypoxic (hypoxemic) preconditioning have been instituted to ameliorate ischemic and reperfusion injury, and means of treating the presumably unavoidable injury pursued only after such injury has been incurred (see, for example, Tong, F., Tang. X., and Liu, D. 2019. “Phycocyanin/PEG-b-(PG-g-PEI) [poly(ethylene glycol)-b-(poly(l-glutamic acid)-g-polyethylenimine] Attenuated Hepatic Ischemia/Reperfusion-induced Pancreatic Islet Injury and Enlarged Islet Functionality,” International Journal of Nanomedicine 14:339-351; Li, D. Y., Liu, W. T., Wang, G. Y., and Shi, X. J. 2018. “Impact of Combined Ischemic Preconditioning and Remote Ischemic Perconditioning on Ischemia-reperfusion Injury after Liver Transplantation,” (Nature Research) Scientific Reports 8(1): 17979; Masola, V., Bellin, G., Vischini, G., Dall'Olmo, L., Granata. S., and 4 others 2018. “Inhibition of Heparanase Protects against Chronic Kidney Dysfunction following Ischemia/Reperfusion Injury,” Oncotarget 2018 9(90): 36185-36201; Masola, V., Zaza, G., Bellin, G., Dall'Olmo, L., Granata. S., and 5 others 2018. “Heparanase Regulates the M1 Polarization of Renal Macrophages and Their Crosstalk with Renal Epithelial Tubular Cells after Ischemia/Reperfusion Injury,” Federation of American Societies for Experimental Biology Journal 32(2): 742-756; Alva, N., Panisello-Roselló, A., Flores, M., Roselló-Catafau, J., and Carbonell. T. 2018. “Ubiquitin-proteasome System and Oxidative Stress in Liver Transplantation,” World Journal of Gastroenterology 24(31): 3521-3530; Ziegler, A, L., Pridgen, T. A., Mills, J. K., Gonzalez, L. M., Van Landeghem, L., Odle, J., and Blikslager, A. T. 2018. “Epithelial Restitution Defect in Neonatal Jejunum is Rescued by Juvenile Mucosal Homogenate in a Pig Model of Intestinal Ischemic Injury and Repair,” Online, PLoS [Public Library of Science] One 13(8): e0200674; Zhou, H., Xie. G., Mao, Y., Zhou, K., Ren, R., and 3 others 2018. “Enhanced Regeneration and Hepatoprotective Effects of Interleukin 22 Fusion Protein on a Predamaged Liver Undergoing Partial Hepatectomy,” Journal of Immunology Research 2018:5241526; Choudhury, R. 2018. “Hypoxia and Hyperbaric Oxygen Therapy: A Review,” International Journal of General Medicine 11:431-442; Francis, A. and Baynosa, R. 2017. “Ischaemia-reperfusion Injury and Hyperbaric Oxygen Pathways: A Review of Cellular Mechanisms,” Diving and Hyperbaric Medicine 47(2): 110-117; Eskandani, M., Vandghanooni, S., Barar. J., Nazemniyeh. H., and Omidi, Y. 2017. “Cell Physiology Regulation by Hypoxia Inducible Factor-1: Targeting Oxygen-related Nanomachineries of Hypoxic Cells,” International Journal of Biological Macromolecules 99:46-62; Pasut, G., Panisello, A., Folch-Puy, E., Lopez, A., Castro-Benitez, C., and 5 others 2016. “Polyethylene Glycols: An Effective Strategy for Limiting Liver Ischemia Reperfusion Injury,” World Journal of Gastroenterology 22(28): 6501-6508; Sanchez-Niño, M. D., Sanz, A. B., and Ortiz, A. 2016. “Chronicity following Ischemia-reperfusion Injury Depends on Tubular-macrophage Crosstalk Involving Two Tubular Cell-derived CSF-1R [colony stimulating factor 1 receptor] Activators: CSF-1 and IL-34 [interleukin 34],” Nephrology, Dialysis, Transplantation 31(9): 1409-1416).

For this reason, choice of the best surgical technique has always taken into consideration the minimization rather than the elimination of ischemie time and reperfusion injury: sustainment of organs in a deceased donor on life support over a longer period with zero ischemic time under normothermic conditions is preferable to ex vivo chilled machine perfusion, and more still than is static or flowing cold storage in a preservative perfusate. As will become apparent, among other benefits, sudden compound vascular bypass, or sudden switched-organ solid organ transplantation also avoids problem bleeding in liver transplantation and the need for cardiopulmonary support of the recipient during heart transplantation, leaving postoperative cognitive impairment if any to be left to anesthesia and/or steroid therapy.

As indicated, in sudden compound vascular bypass, or sudden switched-organ transplantation, solenoid driven vascular valves are used to suddenly switch the donor organ from the circulatory system of the donor to that of the recipient without interruption in the flow of blood. Choice of the heart as the transplant organ is exemplary: the sudden switch technique applies no less to the heart together with the lungs, one or more lungs, kidneys (with or without the pancreas), liver, thymus gland, and pancreas. Unlike the larger solid organs, the tight space for orthotopic may necessitate heterotopic placement.

Citation of the heart serves to emphasize that the blood driver itself can be transplanted thus, and recognizes that heart transplantation has become a standard treatment for end-stage heart failure in adults. As to terminology, ‘open’ and ‘closed’ pertain to the vessel, not to the jacket. Fully ‘open’ denotes that the substrate vessel, or vessel encircled by the jacket is unobstructed, that the diversion chute in the valve is fully retracted, so that blood can flow freely through the substrate ductus with none diverted. Fully ‘closed’ then, means that the vessel is completely blocked off, because the chute is fully extended, diverting all of the blood arriving from upstream to it to another vessel.

No less important, heart transplantation is the only means for establishing normal circulation in neonates and infants bom with a heart so severely malformed that even optimal repair would not establish normal circulation before Fontan circulation, for example, would inevitably lead to multiple organ impairment (see, for example, Egb, A. C., Reddy, Y. N. V., Khan, A. R., Al-Otaibi, M., Akintoye, E., Obokata. M., and Borlaug, B, A. 2018. “Venous Congestion and Pulmonary Vascular Function in Fontan Circulation: Implications for Prognosis and Treatment,” International Journal of Cardiology 271:312-316; Kim, T. H., Yang, H. K., Jang, H. J., Yoo, S., Khalili, K., and Kim, T. K. 2018. “Abdominal Imaging Findings in Adult Patients with Fontan Circulation,” Insights into Imaging 9(3): 357-367; Ohuchi, H., Miyazaki, A., Negishi, J., Hayama, Y., Nakai, M., and 4 others 2017. “Hemodynamic Determinants of Mortality after Fontan Operation,” American Heart Journal 189:9-18; Goldberg, D. J., Surrey, L. F., Glatz, A. C., Dodds, K., O'Byrne, M, L., and 6 others 2017. “Hepatic Fibrosis is Universal following Fontan Operation, and Severity is Associated with Time from Surgery: A Liver Biopsy and Hemodynamic Study,” Journal of the American Heart Association (Oxford, England) 6(5). pii: e004809; Puelz, C., Acosta, S., Rivière, B., Penny, D. J., Brady, K. M., and Rusin, C, G. 2017. “A Computational Study of the Fontan Circulation with Fenestration or Hepatic Vein Exclusion,” Computers in Biology and Medicine 89:405-418; Ozawa, H., Ueno. T., Iwai, S., Kawata, H., Nishigaki, K., Kishimoto, H., and Sawa, Y. 2014. “Contractility-afterload Mismatch in Patients with Protein-losing Enteropathy after the Fontan Operation,” Pediatric Cardiology 35(7): 1225-1231; Yoo, S., Prsa, M., Schantz, D., Grosse-Wortmann, L., Seed, M., Kim, T. K., Wald, R., and Chaturvedi, R. 2014. “M R Assessment of Abdominal Circulation in Fontan Physiology,” International Journal of Cardiovascular Imaging 30(6): 1065-1072; Ohuchi, H., Yasuda, K., Miyazaki, A., Kitano, M., Sakaguchi, H., Yazaki, S., Tsuda, E., and Yamada, O. 2013. “Haemodynamic Characteristics before and after the Onset of Protein Losing Enteropathy in Patients after the Fontan Operation,” European Journal of Cardiothoracic Surgery 43(3): e49-57; Rychik, J., Veldtman, G., Rand, E., Russo, P., Rome, J. J., and 4 others 2012. “The Precarious State of the Liver after a Fontan Operation: Summary of a Multidisciplinary Symposium,” Pediatric Cardiology 33(7): 1001-1012).

Where the organ blood supply and drainage are diffuse, highly branched, as in omenta and mesentery, or plunge deep into the parenchyma, as in the pancreas, the diversion valves are positioned retrograde (upstream, prior to) the arterial branching or entry into the organ or gland, and upstream to the venous branching or penetration, the included blood supply and drainage transplanted as well. For example, along the small intestine, the arterial jacket is placed along the celiac axis or the superior mesenteric artery, and the venous jacket along the superior mesenteric or hepatic portal vein. Essentially, surgical repair of severe heart malformation saves the life of the patient, but that life will be one of poor health, and affecting the development of the brain, most likely deprivation of the potential for significant accomplishment.

While not imperative, not undertaken in conventional heart transplantation, and even beneficial in circumstances where chest pain does not signal the need for immediate medical attention, denervation, the loss of higher center, or extracardiac neural control when the native heart is removed is preferably reinstated. In both conventional merger-type or ‘piggyback’ and second intact heart heterotopic transplantation, the native heart left in place, if carefully avoided, the vagus (previously referred to as the pneumogastric) and sympathetic cardiac nerves are substantially unaffected.

In a retransplant, these must be transected but can be surgically reconnected or anastomosed, this neurorrhaphy accomplished with the aid of fibrin, laser-activated chitosan, or photocrosslinkable gelatin/tropoelastin hydrogel adhesive, 10-0 monofilament microsurgical sutures with platelet rich plasma, melatonin, and/or dexamethasone, by reconstruction using harvested nerve fibers, or anastomosis combined with regenerative promotive measures (see, for example, Boecker, A., Daeschler, S. C., Kneser, U., and Harhaus, L. 2019. “Relevance and Recent Developments of Chitosan in Peripheral Nerve Surgery,” Online, Frontiers in Cellular Neuroscience 13:104; Duraikannu. A., Krishnan, A., Chandrasekhar, A., and Zochodne, D. W. 2019. “Beyond Trophic Factors: Exploiting the Intrinsic Regenerative Properties of Adult Neurons,” Online, Frontiers in Cellidar Neuroscience 13:128; Neumann, B., Linton, C., Giordano-Santini. R., and Hilliard, M, A. 2019. “Axonal Fusion: An Alternative and Efficient Mechanism of Nerve Repair,” Progress in Neurobiology 173:88-101; Rui, J., Zhou, Y. J., Zhao, X., Li, J. F., Gu. Y. D., and Lao, J. 2019. “Endogenous Automatic Nerve Discharge Promotes Nerve Repair: An Optimized Animal Model,” Neural Regeneration Research 14(2): 306-312; Sahar, M. S. U., Barton, M., and Tansley, G, D. 2019. “Bridging Larger Gaps in Peripheral Nerves Using Neural Prosthetics and Physical Therapeutic Agents,” Neural Regeneration Research 14(7): 1109-1115: Tang, B. L. 2019. “Why is NMNAT [nicotinamide mononucleotide adenylyltransferase] Protective against Neuronal Cell Death and Axon Degeneration, but Inhibitory of Axon Regeneration?,” Cells 8(3). pii: E267; Tuna Edizer, D., Dönmez, Z., Gül. M., Yiğit, O., Yiğitcan, B., Adatepe, T., and Uzun, N. 2019. “Effects of Melatonin and Dexamethasone on Facial Nerve Neurorrhaphy,” Journal of International Advanced Otology 15(1): 43-50; Zhang, P. X., Han, N., Kou, Y. H., Zhu, Q. T., Liu, X, L., and 3 others 2019. “Tissue Engineering for the Repair of Peripheral Nerve Injury,” Neural Regeneration Research 14(1): 51- 58; Vijayavenkataraman, S., Thaharah, S., Zhang, S., Lu, W. F., and Fuh, J. Y, H. 2019. “3D-Printed PCL/rGO [polycaprolactone/reduced graphene oxide] Conductive Scaffolds for Peripheral Nerve Injury Repair,” Artificial Organs 43(5): 515-523; Wang. D., Wang. Y., Tian, W., and Pan, J. 2019. “Advances of Tooth-derived Stem Cells in Neural Diseases Treatments and Nerve Tissue Regeneration,” Cell Proliferation 52(3): e12572; Alsmadi, N. Z., Bendale, G. S., Kanneganti, A., Shihabeddin. T., Nguyen, A. H., and 5 others 2018. “Glial-derived Growth Factor and Pleiotrophin Synergistically Promote Axonal Regeneration in Critical Nerve Injuries,” Acta Biomaterialia 78:165-177; An, S., Zhou, M., Li, Z., Feng. M., Cao, G., Lu, S., and Liu, L. 2018. “Administration of CoCl2 [cobalt chloride] Improves Functional Recovery in a Rat Model of Sciatic Nerve Transection Injury,” International Journal of Medical Sciences 15(13): 1423-1432; Bendella, H., Rink, S., Grosheva, M., Sarikcioglu, L., Gordon, T., and Angelov, D. N. 2018. “Putative Roles of Soluble Trophic Factors in Facial Nerve Regeneration, Target Reinnervation, and Recovery of Vibrissal Whisking,” Experimental Neurology 300:100-110; Huang, L., Zhu, L., Shi, X., Xia, B., Liu, Z., and 7 others 2018. “A Compound Scaffold with Uniform Longitudinally Oriented Guidance Cues and a Porous Sheath Promotes Peripheral Nerve Regeneration in Vivo,” Acta Biomaterialia 68:223-236; Rosso, M. P. O., Buchaim, D. V., Kawano, N., Furlanette, G., Pomini, K. T., and Buchaim, R. L. 2018. “Photobiomodulation Therapy (PBMT) in Peripheral Nerve Regeneration: A Systematic Review,” Bioengineering (Basel, Switzerland) 5(2). pii: E44; Sanchez-Gonzalez, S., Diban, N., Bianchi, F., Ye. H., and Urtiaga, A. 2018. “Evidences of the Effect of GO [graphene oxide] and rGO [reduced graphene oxide] in PCL [poly(ε-caprolactone] Membranes on the Differentiation and Maturation of Human Neural Progenitor Cells,” Macromolecular Bioscience 18(11): e1800195; Sarker, M. D., Naghieh, S., McInnes, A. D., Schreyer, D. J., and Chen, X. 2018. “Regeneration of Peripheral Nerves by Nerve Guidance Conduits: Influence of Design, Biopolymers, Cells, Growth Factors, and Physical Stimuli,” Progress in Neurobiology 171:125-150; Sarker, M., Naghieh, S., McInnes, A. D., Schreyer, D. J., and Chen, X. 2018. “Strategic Design and Fabrication of Nerve Guidance Conduits for Peripheral Nerve Regeneration,” Biotechnology Journal 13(7): e1700635; Sohn, E. J. and Park, H. T. 2018. “MicroRNA Mediated Regulation of Schwann Cell Migration and Proliferation in Peripheral Nerve Injury,” Online, BioMed Research International 2018:8198365; Soucy, J. R., Shirzaei Sani, E., Portillo Lara, R., Diaz, D., Dias. F., and 4 others 2018. “Photocrosslinkable Gelatin/Tropoelastin Hydrogel Adhesives for Peripheral Nerve Repair,” Tissue engineering. Part A 24(17-18): 1393-1405; Teoh, J. S., Wong, M. Y., Vijayaraghavan. T., and Neumann, B. 2018. “Bridging the Gap: Axonal Fusion Drives Rapid Functional Recovery of the Nervous System,” Neural Regeneration Research 13(4): 591-594; Wang. Y., Pan, J., Wang, D., and Liu, J. 2018. “The Use of Stem Cells in Neural Regeneration: A Review of Current Opinion,” Current Stem Cell Research and Therapy 13(7): 608-617; Zhang, J. Liu. Y., and Lu, L. 2018. “Emerging Role of MicroRNAs in Peripheral Nerve System,” Life Sciences 207:227-233; Barton M. J., John, J. S., Clarke, M., Wright, A., and Ekberg, J. 2017. “The Glia Response after Peripheral Nerve Injury: A Comparison between Schwann Cells and Olfactory Ensheathing Cells and Their Uses for Neural Regenerative Therapies,” Online, MDPI [Multidisciplinary Digital Publishing Institute] International Journal of Molecular Sciences (Basel, Switzerland) 18(2). pii: E287; Du. J., Liu, J., Yao. S., Mao. H., Peng. J., and 7 others 2017. “Prompt Peripheral Nerve Regeneration Induced by a Hierarchically Aligned Fibrin Nanofiber Hydrogel,” Acta Biomaterialia 55:296-309; Rochkind, S. 2017. “Photobiomodulation in Neuroscience: A Summary of Personal Experience,” Photomedicine and Laser Surgery 35(11): 604-615; Yu, Q., Zhang, S. H., Wang. T., Peng, F., Han, D., and Gu. Y, D. 2017. “End-to-side Neurorrhaphy Repairs Peripheral Nerve Injury: Sensory Nerve Induces Motor Nerve Regeneration,” Neural Regeneration Research 12(10): 1703-1707; Liu, F., Lin. H., and Zhang, C. 2016. “Construction of Tissue-engineered Nerve Conduits Seeded with Neurons Derived from Hair-follicle Neural Crest Stem Cells,” Methods in Molecular Biology (Clifton, New Jersey) 1453:33-38; Mandelbaum-Livnat, M. M., Almog, M., Nissan, M., Loeb, E., Shapira. Y., and Rochkind, S. 2016. “Photobiomodulation Triple Treatment in Peripheral Nerve Injury: Nerve and Muscle Response,” Photomedicine and Laser Surgery 34(12): 638-645; Qin, J., Wang, L., Sun, Y., Sun, X., Wen, C., Shahmoradi, M., and Zhou, Y. 2016. “Concentrated Growth Factor Increases Schwann Cell Proliferation and Neurotrophic Factor Secretion and Promotes Functional Nerve Recovery in Vivo,” International Journal of Molecular Medicine 37(2): 493-500; Schuh, C. M., Hausner, T., and Redl. H, R. 2016. “A Therapeutic Shock Propels Schwann cells to Proliferate in Peripheral Nerve Injury,” Brain Circulation 2(3): 138-140; Buchaim, R, L., Andreo. J. C., Barraviera, B., Ferreira Junior, R. S., Buchaim, D. V., and 3 others 2015. “Effect of Low-level Laser Therapy (LLLT) on Peripheral Nerve Regeneration Using Fibrin Glue Derived from Snake Venom,” Injury 46(4): 655-660; Barton, M. J., Morley, J. W., Stoodley, M. A., Lauto, A., and Mahns. D, A. 2014. “Nerve Repair: Toward a Sutureless Approach,” Neurosurgical Review 37(4): 585-595: Casiraghi, F., Perico, N., and Remuzzi, G. 2013. “Mesenchymal Stromal Cells to Promote Solid Organ Transplantation Tolerance,” Current Opinion in Organ Transplantation 18(1): 51-58; Liu, J., Wang, J., Luo, F., Wang, Z., and Wang, Y. 2013. “Reconstruction of the Abdominal Vagus Nerve Using Sural Nerve Grafts in Canine Models,” Online, PLoS [Public Library of Science] One 8(3): e58903; Samara, C., Poirot, O., Domenech-Estevez, E., and Chrast, R. 2013. “Neuronal Activity in the Hub of Extrasynaptic Schwann Cell-Axon Interactions,” Online, Frontiers in Cellular Neuroscience 7:228; Li, W., Chen, Z., Wu. R., Zhang. W., and Lu, C. 2012. “Protection and Functional Repair of Vagus Nerve during the Operation of Cervical Vagal Paraganglioma,” (in Chinese with English abstract at Pubmed), Lin Chuang er Bi Yan Hou Tou Jing Wai Ke Za Zhi [Journal of Clinical Otorhinolaryngology, Head, and Neck Surgery] 26(16): 721-724; Turgut, M. and Kaplan, S. 2011. “Effects of Melatonin on Peripheral Nerve Regeneration,” Recent Patents on Endocrine, Metabolic, and Immune Drug Discovery 5(2): 100-108; Deumens. R., Bozkurt, A., Meek, M. F., Marcus, M. A., Joosten, E. A., Weis, J., and Brook, G, A. 2010. “Repairing Injured Peripheral Nerves: Bridging the Gap,” Progress in Neurobiology 92(3): 245-276; Gordon, T. 2010. “The Physiology of Neural Injury and Regeneration: The Role of Neurotrophic Factors,” Journal of Communication Disorders 43(4): 265-273; Siemionow, M., Bozkurt, M., and Zor, F. 2010. “Regeneration and Repair of Peripheral Nerves with Different Biomaterials: Review,” Microsurgery 30(7): 574-588; Gordon, T. 2009. “The Role of Neurotrophic Factors in Nerve Regeneration,” Neurosurgical Focus 26(2): E3; Lauto, A., Foster, L., Avolio, A., Sampson, D., Raston, C., and 3 others 2008. Sutureless Nerve Repair with Laser-activated Chitosan Adhesive: A Pilot in Vivo Study,” Photomedicine and Laser Surgery 26(3): 227-234; Bregeon, F., Alliez, J. R., Hery, G., Marqueste, T., Ravailhe, S., and Jammes, Y. 2007. “Motor and Sensory Re-innervation of the Lung and Heart after Re-anastomosis of the Cervical Vagus Nerve in Rats,” Journal of Physiology 581 (Part 3): 1333-1140; Dezawa, M. 2002. “Central and Peripheral Nerve Regeneration by Transplantation of Schwann Cells and Transdifferentiated Bone Marrow Stromal Cells,” Anatomical Science International 77(1): 12-25; Farrag, T. Y., Lehar, M., Verhaegen, P., Carson, K. A., and Byme, P. J. 2007. “Effect of Platelet Rich Plasma and Fibrin Sealant on Facial Nerve Regeneration in a Rat Model,” Laryngoscope 117(1): 157-165; Gordon, T., Brushart, T. M., Amirjani, N., and Chan, K. M. 2007. “The Potential of Electrical Stimulation to Promote Functional Recovery after Peripheral nerve Injury-Comparisons between Rats and Humans,” Acta Neurochirurgica. Supplement 100:3-11; Suri, A., Mehta, V. S., and Sarkar, C. 2002. “Microneural Anastomosis with Fibrin Glue: An Experimental Study,” Neurology India 50(1): 23-26; Dezawa, M. 2000. “The Interaction and Adhesive Mechanisms between Axon and Schwann Cell during Central and Peripheral Nerve Regeneration,” Kaibogaku Zasshi [Journal of Anatomy] 75(3): 255-265; Dezawa, M., Mutoh, T., Dezawa, A., and Adachi-Usami, E. 1998. “Putative Gap Junctional Communication between Axon and Regenerating Schwann Cells during Mammalian Peripheral Nerve Regeneration,” Neuroscience 85(3): 663-667; Kodama, M., Arakawa, A., Ito, M., and Koyama, K. 1997. “The Effects of Convenient Vagorrhaphy on the Early Recovery of Gastric Secretion and Emptying: An Experimental Study on Function-preserving Gastric Cancer Surgery,” Surgery Today 27(8): 741-744; Wikholm, R. P., Swett, J. E., Torigoe, Y., and Blanks, R, H. 1988. “Repair of Severed Peripheral Nerve: A Superior Anatomic and Functional Recovery with a New “Reconnection” Technique,” Otolaryngology-Head and Neck Surgery 99(4): 353-361; Speidel, C, C. 1946. “Prolonged Histories of Vagus Nerve Regeneration Patterns, Sterile Distal Stumps, and Sheath Cell Outgrowths,” Anatomical Record 94:499; Tuckett, I. L. 1900. “Note on the Regeneration of the Vagus Nerve,” Journal of Physiology 25(4): 303-305).

The central barriers to the restoration of sight through transplantation of the eyes are 1. The elusiveness of optic nerve regeneration even when autologous, 2. The risk of rejection, and 3. Indecisiveness as to the best surgical technique to use. These draw much research activity; however, much of this work is devoted to autologous regeneration of the optic nerve and the recovery of vision following traumatic injury to either or both optic nerves rather than to allogeneic regeneration to allow transplantation, or whole-eye transplantation, where the optic nerves are not damaged but completely transected.

### Compound Vascular Bypass as Promoting Whole Eye Allotransplantation Through Uninterrupted Perfusion of the Retina

The problem of whole-eye transplantation is also distinct from replacement of a portion of the eye, such as in corneal transplantation. As in organ transplantation generally, allowing the donor to die rather than harvesting the organ or organs and performing the transplant only once it is clear that the donor is certain to die militates against the odds for success. Because it harvests the graft eye or eyes while the donor is effectively still alive, metered compound vascular bypass, or sudden switched-organ transplantation should materially reduce the hindrances that obstruct this capability.

The use of basic side-entry or servovalve-jackets to deliver one or more immunosuppressants and a minimal dose of a potentially neurotoxic but at the same time immunosuppressant neuroregenerative calcineurin inhibitor such as tacrolimus (fujimycin, FK506) by direct release into the internal carotids would eliminate much of the adverse side effects and resistance to coaptation of nervous tissue, and in so doing, overcome two of the three major hindrances to eye transplantation (Bourne, D., Li, Y., Komatsu, C., Miller, M. R., Davidson, E. H., and 7 others 2017. “Whole-eye Transplantation: A Look into the Past and Vision for the Future,” Eve (London, England) 31(2): 179-184).

Whether tacrolimus has the potential to impair the blood brain barrier indirectly by elevating the blood pressure, or directly, or both, the addition of an antihypertensive, mannitol, and an anti-inflammatory, such as methylprednisolone, for direct release into the internal carotids should alleviate the intracranial hypertension beyond that achieved through administration of a systemic antihypertensive alone and so expedite noninjurious passage through the blood brain barrier. The unwanted side effects of the immunosuppressive drugs commonly used in and following transplantation are delineated above in section 2b(a), entitled Sudden Compound Vascular Bypass, or Sudden Switched-organ Transplantation.

Additionally, should the need arise, vascular servovalves make it possible to reduce the intracranial blood pressure by continuously diverting a portion of flow through the internal carotids to another larger vessel such as back down into the descending aorta, and this diversion can be automatically adjusted dynamically in response to pressure sensor data fed back to the fully implanted prosthetic disorder control system microcontroller or microprocessor.

Resolution of the encephalopathy automatically stops the diversion. The reversible encephalopathy which can result from tacrolimus and related matters are well documented (Viñuela-Berni, V., Gómez-González, B., and Quintanar-Stephano, A. 2020. “Blockade of Arginine Vasopressin Receptors Prevents Blood-brain Barrier Breakdown in Experimental Autoimmune Encephalomyelitis,” (Nature Research) Scientific Reports 10(1): 467; Largeau, B., Le Tilly, O., Sautenet, B., Salmon Gandonnière, C., Barin-Le Guellec, C. and Ehrmann, S. 2019. “Arginine Vasopressin and Posterior Reversible Encephalopathy Syndrome Pathophysiology: The Missing Link?,” Molecular Neurobiology 56(10): 6792-6806; Meyer, M., Niemoller, U., Stein, T., Schmetsdorf, S., Arnold, A., and 5 others 2019. “Positive Effect of Steroids in Posterior Reversible Encephalopathy Syndrome,” Case Reports in Neurology 11(2): 173-177; Starcea, M., Gavrilovici, C., Munteanu, M., and Miron, I. 2018. “Focal Segmental Glomerulosclerosis in Children Complicated by Posterior Reversible Encephalopathy Syndrome,” Journal of International Medical Research 46(3): 1172-1177; Fischer, M. and Schmutzhard, E. 2017. “Posterior Reversible Encephalopathy Syndrome,” Journal of Neurology 264(8): 1608-1616; Gavrilovici, C., Miron, I., Voroneanu, L., Badarau, S., and Starcea, M. 2017. “Posterior Reversible Encephalopathy Syndrome in Children with Kidney Disease,” International Urology and Nephrology 49(10): 1793-1800; Song, T., Rao, Z., Tan, Q., Qiu, Y., Liu, J., and 3 others 2016. “Calcineurin Inhibitors Associated Posterior Reversible Encephalopathy Syndrome in Solid Organ Transplantation: Report of 2 Cases and Literature Review,” Medicine (Baltimore, Maryland) 95(14): e3173; Granata, G., Greco, A., lannella, G., Granata, M., Manno, A., Savastano, E., and Magliulo, G. 2015. “Posterior Reversible Encephalopathy Syndrome-Insight into Pathogenesis, Clinical Variants and Treatment Approaches,” Autoimmunity Reviews 14(9): 830-836; Zeynalov, E., Jones, S. M., Seo, J. W., Snell, L. D., and Elliott, J. P. 2015. “Arginine-Vasopressin Receptor Blocker Conivaptan Reduces Brain Edema and Blood-Brain Barrier Disruption after Experimental Stroke in Mice,” Online, PLoS [Public Library of Science] One 10(8): e0136121; Ishikura, K., Hamasaki, Y., Sakai, T., Hataya, H., Mak, R. H., and Honda, M. 2012. “Posterior Reversible Encephalopathy Syndrome in Children with Kidney Diseases,” Pediatric Nephrology (Berlin, Germany) 27(3): 375-384; Kanekiyo, T., Hara, J., Matsuda-Hashii, Y., Fujisaki, H., Tokimasa, S., and 5 others 2005. “Tacrolimus-related Encephalopathy following Allogeneic Stem Cell Transplantation in Children,” International Journal of Hematology 81(3): 264-268; Kaczmarek, I., Groetzner, J., Meiser, B., Mueller, M., Landwehr, P., and 3 others 2003. “Impairment of the Blood-brain Barrier Can Result in Tacrolimus-induced Reversible Leucoencephalopathy following Heart Transplantation,” Clinical Transplantation (Copenhagen, Denmark) 17(5): 469-472; Tyagi, M, G. and Thomas, M. 1999. “Enhanced Cardiovascular Reactivity to Desmopressin in Water-restricted Rats: Facilitatory Role of Immunosuppression,” Methods and Findings in Experimental and Clinical Pharmacology 21(9): 619-624).

The key obstacle to whole eye transplantation is that the optic nerves are extensions or projections of the brain itself, integral to the sight-supporting tissue that continues caudal to the visual center in the occipital lobe so that transection at any intermediate level might preclude the restoration of sight (see, for example, Huff, T., Mahabadi, N., and Tadi, P. 2024. “Neuroanatomy, Visual Cortex,” Treasure Island, Florida: StatPearls Publishing Co; Sanchez Lopez de Nava, A., Somani, A. N., and Salini, B. 2023. “Physiology, Vision,” Online, Treasure Island, Florida: StatPearls Publishing Co.: Gupta, M., Ireland, A. C., and Bordoni, B. 2022. “Neuroanatomy, Visual Pathway,” Online, Treasure Island, Florida: StatPearls Publishing Co.; Harris, J. M. and Scott, J. (eds.) 2012. Visual Cortex: Anatomy, Functions and Injuries. Hauppauge, New York: Nova Science Publishing, Inc.).

As to the third impediment, surgical technique, rather than to transect the optic nerves, now known to preclude the restoration of vision using existing means, transplantation that preserves the retina and optic nerve intact by extending the graft back to or partially into the thalamus with stem cell and immune tolerogenic medication would allow it to be determined whether the optic nerves themselves are exceptionally refractory to anastomosis as all neural tissue is not. Such would appear to be distinctly against the odds; however, if surprisingly true or treatable to make it work, this would reduce the difficulty in achieving donor-recipient transplanted nerve fusion or coaptation, possibly making it possible for the globes to elude rejection.

For the time being, the reality is that a couple centuries of attempts notwithstanding, whole-eye transplantation in mammals remains elusive. Whole eye allotransplantation still conjectural, applications of vascularized composite tissue allotransplantation which have actually been implemented are addressed below in section 2b(1)(b)(b12), entitled Metered Compound Vascular Bypass. or Switched-organ, Vascularized Composite Tissue Allotransplantation. If realized, it is probable that because the retina is thoroughly perfused, the vessels of the choroidal and retinal networks, extremely fine as to deteriorate quickly in a stoppage of blood flow, use of the compound vascular bypass transfer method would maintain perfusion throughout the replacement procedure to eliminate even a moment of nonperfusion. If so, this technique will have participated as central in the realization of whole eye allotransplantation.

That to allow the retina to go unperfused even for seconds would prove ruinous is to be expected (Brydges, H. T., Onuh, O. C., Chaya, B. F., Tran, D, L., Cassidy, M. F., and 3 others 2023. “Combined Face and Whole Eye Transplantation: Cadaveric Rehearsals and Feasibility Assessment,” Online, Plastic and Reconstructive Surgery. Global Open (Philadelphia, Pennsylvania) 16:11(11): e5409; Laspro. M., Chaya, B. F., Brydges, H. T., Dave. N., Thys. E., and 5 others 2023. “Technical Feasibility of Whole-eye Vascular Composite Allotransplantation: A Systematic Review,” Online, Plastic and Reconstructive Surgery. Global Open (Philadelphia, Pennsylvania) 11(4): e4946; Hare, E. 2016. “An Audacious Vision: Researchers Aim for First Human Eye Transplant with the Decade,” Stat (Boston, Massachusetts), Online, at statnews.com/2016/11/23).

Beginning one or two years after a heart transplant, the vagus, as is the optic also a cranial nerve, appears to fuse and begin to function, stirring hope that the same will apply to the optic nerve (see, for example, Zor, F., Karagoz, H., Kapucu, H., Kulahci, Y., Janjic. J. M., and Gorantla. V. S. 2019. “Immunological Considerations and Concerns as Pertinent to Whole Eye Transplantation,” Current Opinion in Organ Transplantation (Hagerstown, Maryland) 24(6): 726-732; Zor, F., Polat, M., Kulahci, Y., Sahin, H., Aral, A. M., and 5 others 2019. “Demonstration of Technical Feasibility and Viability of Whole Eye Transplantation in a Rodent Model,” Journal of Plastic, Reconstructive and Aesthetic Surgery (Amsterdam, North Holland, Netherlands) 72(10): 1640-1650; Chen, W., Noori, J., He, L., Komatsu, C., Miller, M. and 9 others 2018. “Abstract 37: Whole Eye Allograft Transplantation and Immunosuppression: Survival of The Transplanted Rodent Eye Using Tacrolimus Monotherapy,” Plastic and Reconstructive Surgery-Global Open 6(4S): 29-30; Siemionow, M., Bozkurt, M., Zor, F., Kulahci, Y., Uygur. S., and 3 others 2018. “A New Composite Eyeball-Periorbital Transplantation Model in Humans: An Anatomical Study in Preparation for Eyeball Transplantation,” Plastic and Reconstructive Surgery 141(4): 1011-1018; Stern, J. H., Tian, Y., Funderburgh, J., Pellegrini, G., Zhang, K., and 5 others 2018. “Regenerating Eye Tissues to Preserve and Restore Vision,” Cell Stem Cell 22(6): 834-849; Benowitz, L. I., He, Z., and Goldberg. J. L. 2017. “Reaching the Brain: Advances in Optic Nerve Regeneration,” Experimental Neurology 287 (Part 3): 365-373; Bourne, D., Li, Y., Komatsu, C., Miller, M. R., Davidson, E. H., and 7 others 2017. Op cit. preceding paragraph; Chun, B. Y. and Cestari, D. M. 2017. “Advances in Experimental Optic Nerve Regeneration,” Current Opinion in Ophthalmology 28(6): 558-563; Li, H. J., Sun, Z, L., Yang, X. T., Zhu, L., and Feng, D, F. 2017. “Exploring Optic Nerve Axon Regeneration,” Current Neuropharmacology 15(6): 861-873; Yungher, B. J., Ribeiro, M., and Park, K. K. 2017. “Regenerative Responses and Axon Pathfinding of Retinal Ganglion Cells in Chronically Injured Mice,” Investigative Ophthalmology and Visual Science 58(3): 1743-1750; Davidson, E. H., Wang, E. W., Yu, J. Y., Fernandez-Miranda, J. C., Wang, D. J., and 8 others 2016. “Clinical Considerations for Vascularized Composite Allotransplantation of the Eye,” Journal of Craniofacial Surgery 27(7): 1622-1628; Davidson, E. H., Wang, E. W., Yu, J. Y., Fernandez-Miranda, J. C., Wang, D. J., and 4 others 2016. “Total Human Eye Allotransplantation: Developing Surgical Protocols for Donor and Recipient Procedures,” Plastic and Reconstructive Surgery 138(6): 1297-1308; Dhamodaran, K., Subramani, M., Ponnalagu, M., Shetty, R., and Das, D. 2014. “Ocular Stem Cells: A Status Update!,” Stem Cell Research and Therapy 5(2): 56; Benowitz, L. I. and Yin, Y. 2010. “Optic Nerve Regeneration,” Archives of Ophthalmology (Chicago, Illinois) 128(8): 1059-1064; Ellenberg, D., Shi, J., Jain, S., Chang, J. H., Ripps, H., and 9 others 2009. “Impediments to Eye Transplantation: Ocular Viability following Optic-nerve Transection or Enucleation,” British Journal of Ophthalmology 93(9): 1134-1140). Until whole-eye transplantation becomes a reality, patients with retinitis pigmentosa or age-related macular degeneration might obtain a measure of vision restoration through a retinal implant.

Unlike some remedies effective in lower forms which prove to be ineffective in man, nerve regenerative phenomena in murids appears applicable to man (Teoh, J. S., Wong, M. Y., Vijayaraghavan, T., and Neumann, B. 2018. Op cit. above in this section; Teoh, J. S., Wong, M. Y., Vijayaraghavan, T., and Neumann, B. 2018, Op cit. above in this section; Gordon, T. and Borschel, G, H. 2017. “The Use of the Rat as a Model for Studying Peripheral Nerve Regeneration and Sprouting after Complete and Partial Nerve Injuries,” Experimental Neurology 287 (Part 3): 331-347; Gordon, T. Brushart. T. M., Amirjani, N. and Chan, K. M. 2007, Op cit., preceding paragraph). Detailed applications of electrical stimulation may emerge as misconceived (Srinivasan, S., Vyas, K., McAvoy, M., Calvaresi, P., Khan, O. F., and 3 others 2019. “Polyimide Electrode-based Electrical Stimulation Impedes Early Stage Muscle Graft Regeneration,” Online, Frontiers in Neurology 10:252).

Healing of a neurorrhaphy may be expedited through the application of electrical stimulation (Hu. M., Hong, L., Liu, C., Hong, S., He. S., and 3 others 2019. “Electrical Stimulation Enhances Neuronal Cell Activity Mediated by Schwann Cell Derived Exosomes,” Science Reports 9(1): 4206; Zheng, Z., Huang, L., Yan, L., Yuan, F., Wang, L., and 4 others 2019. “Polyaniline Functionalized Graphene Nanoelectrodes for the Regeneration of PC12 Cells [neural crest-derived mixture of neuroblastic and eosinophilic cells] via Electrical Stimulation,” Online, MDPI [Multidisciplinary Digital Publishing Institute] International Journal of Molecular Sciences (Basel, Switzerland) 20(8). pii: E2013; McGregor, C, E. and English, A. W. 2019. “The Role of BDNF [brain-derived neurotrophic factor] in Peripheral Nerve Regeneration: Activity-dependent Treatments and Val66 Met [a single nucleotide polymorphism in the BDNF gene],” Online, Frontiers in Cellular Neuroscience 12:522; Wang, J., Cheng. Y., Chen, L., Zhu, T., Ye, K., and 5 others 2019. “In Vitro and in Vivo Studies of Electroactive Reduced Graphene Oxide-modified Nanofiber Scaffolds for Peripheral Nerve Regeneration,” Acta Biomaterialia 84:98-113; Sliow. A., Ma, Z., Gargiulo, G., Mahns. D., Mawad, D., and 10 others 2019. “Stimulation and Repair of Peripheral Nerves Using Bioadhesive Graft-antenna,” Advanced Science (Weinheim, Baden-Württemberg, Germany) 6(11): 1801212; Bendella, H., Rink, S., Grosheva, M., Sarikcioglu, L., Gordon, T., and Angelov, D. N. 2018, Op cit., above in this section; Mendez, A., Hopkins, A., Biron, V, L., Seikaly, H., Zhu, L. F., and Cote, D. W. J. 2018. “Brief Electrical Stimulation and Synkinesis after Facial Nerve Crush Injury: A Randomized Prospective Animal Study,” Journal of Otolaryngology-Head and Neck Surgery 47(1): 20; Zhang, Z., Klausen, L. H., Chen, M., and Dong. M. 2018. “Electroactive Scaffolds for Neurogenesis and Myogenesis: Graphene-based Nanomaterials,” Small (Weinheim an der Bergstrasse, Germany) 14(48): e1801983; Chan, K. M., Curran, M. W., and Gordon, T. 2016. “The Use of Brief Post-surgical Low Frequency Electrical Stimulation to Enhance Nerve Regeneration in Clinical Practice,” Journal of Physiology 594(13): 3553-3559; Gordon, T. 2016. “Electrical Stimulation to Enhance Axon Regeneration after Peripheral Nerve Injuries in Animal Models and Humans,” Neurotherapeutics 13(2): 295-310; Gordon, T. 2016. Nerve Regeneration: Understanding Biology and its Influence on Return of Function after Nerve Transfers,” Hand Clinics 32(2): 103-117; Gordon, T. 2016. “Nerve Regeneration in the Peripheral and Central Nervous Systems,” Journal of Physiology 594(13): 3517-3520; Mendez, A., Seikaly, H., Biron, V, L., Zhu, L. F., and Cote, D. W. 2016. “Brief Electrical Stimulation after Facial Nerve Transection and Neurorrhaphy: A Randomized Prospective Animal Study,” Journal of Otolaryngology-Head and Neck Surgery 45:7; Lin, Y. C., Kao, C. H., Chen, C. C., Ke, C. J., Yao, C. H., and Chen, Y. S. 2015. “Time-course Effect of Electrical Stimulation on Nerve Regeneration of Diabetic Rats,” Online, PLoS [Public Library of Science] One 10(2): e0116711; Anderson, M., Shelke, N. B., Manoukian, O. S., Yu, X., Mccullough, L. D., and Kumbar, S, G. 2015. “Peripheral Nerve Regeneration Strategies: Electrically Stimulating Polymer Based Nerve Growth Conduits,” Critical Reviews in Biomedical Engineering 43(2-3): 131-159; Elzinga, K., Tyreman, N., Ladak, A., Savaryn, B., Olson, J., and Gordon, T. 2015. “Brief Electrical Stimulation Improves Nerve Regeneration after Delayed Repair in Sprague Dawley Rats,” Experimental Neurology 269:142-153; Willand, M. P. 2015. “Electrical Stimulation Enhances Reinnervation after Nerve Injury,” European Journal of Translational Myology 25(4): 243-248; Calvey, C., Zhou, W., Stakleff, K. S., Sendelbach-Sloan, P., Harkins, A. B., Lanzinger, W., and Willits, R. K. 2015. “Short-term Electrical Stimulation to Promote Nerve Repair and Functional Recovery in a Rat Model,” Journal of Hand Surgery 40(2): 314-322; Lin, Y. C., Kao, C. H., Cheng, Y. K., Chen, J. J., Yao, C. H., and Chen, Y. S. 2014. “Current-modulated Electrical Stimulation as a Treatment for Peripheral Nerve Regeneration in Diabetic Rats,” Restorative Neurology and Neuroscience 32(3): 437-446; Gordon, T., Amirjani, N., Edwards, D. C., and Chan, K. M. 2010. “Brief Post-surgical Electrical Stimulation Accelerates Axon Regeneration and Muscle Reinnervation Without Affecting the Functional Measures in Carpal Tunnel Syndrome Patients,” Experimental Neurology 223(1): 192-202; Hadlock, T., Lindsay, R., Edwards, C., Smitson, C., Weinberg, J., Knox, C., and Heaton, J. T. 2010. “The Effect of Electrical and Mechanical Stimulation on the Regenerating Rodent Facial Nerve,” Laryngoscope 120(6): 1094-102; Gordon, T., Udina, E., Verge, V. M., and de Chaves, E. I. 2009. “Brief Electrical Stimulation Accelerates Axon Regeneration in the Peripheral Nervous System and Promotes Sensory Axon Regeneration in the Central Nervous System,” Motor Control 13(4): 412-441; Gordon, T., Sulaiman, O. A., and Ladak, A. 2009. Chapter 24: Electrical Stimulation for Improving Nerve Regeneration: Where Do We Stand?,” International Review of Neurobiology 87:433-444; Gordon, T., Brushart, T. M., and Chan, K. M. 2008. “Augmenting Nerve Regeneration with Electrical Stimulation,” Neurological Research 30(10): 1012-1022; Gordon, T, Brushart, T. M., Amirjani, N. and Chan, K. M. 2007. “The Potential of Electrical Stimulation to Promote Functional Recovery after Peripheral Nerve Injury-Comparisons between Rats and Humans,” Acta Neurochirurgica Supplement 100:3-11; Brushart, T. M., Jari, R., Verge, V., Rohde, C., and Gordon, T. 2005. “Electrical Stimulation Restores the Specificity of Sensory Axon Regeneration,” Experimental Neurology 194(1): 221-229; Nix, W, A. and Hopf, H, C. 1983. “Electrical Stimulation of Regenerating Nerve and Its Effect on Motor Recovery,” Brain Research 272(1): 21-25).

Interrelated medical topics such as tolerogenic induction therapy, donor-recipient cross-chimerization, transplant organ medicinal support, and the treatment of post transplantation adverse alloreactions the same in essence as with conventional transplantation, the matters addressed here are confined to the technique and means for accomplishing a sudden compound vascular bypass, or sudden switched-organ transplantation. In metered, as opposed to sudden compound vascular bypass, or sudden switched-organ transplantation, switching is not sudden but rather extended over an interval, during which recipient/donor reciprocal cross-circulation is allowed to initiate the induction of immune tolerance.

### Compound Vascular Bypass Heart Transplantation

Using switch transplantation, total orthotopic heart transplantation replaces the heart with its arterial and venous vessel stumps without entry into the heart as non-open heart surgery. More specifically, total orthotopic transplantation as here contemplated involves removal of both recipient and donor hearts with vessels, entirely-without leaving portions of the recipient atria through which the pulmonary arteries are connected as in a conventional ‘total’ orthotopic heart transplantation, whereby the donor, but not the recipient heart, is removed in its entirety (see, for example, Dreyfus, G., Jebara, V., Mihaileanu, S., and Carpentier, A, F. 1991. “Total Orthotopic Heart Transplantation: An Alternative to the Standard Technique,” Annals of Thoracic Surgery 52(5): 1181-1184).

Total removal of both donor and recipient hearts, for example, each intact, allows greater stump length of the pulmonary veins, allowing readier access to these, and the incisions away from the heart itself, facilitating the placement of the valve-jackets, reducing the risk of postprocedural dysrhythmias as well as facilitating any later need for heart surgery or retransplantation (see, for example, Rivinius, R., Helmschrott, M., Ruhparwar, A., Erbel, C., Gleissner, C. A., and 5 others 2017. “The Influence of Surgical Technique on Early Posttransplant Atrial Fibrillation-Comparison of Biatrial, Bicaval, and Total Orthotopic Heart Transplantation,” Therapeutics and Clinical Risk Management 13:287-297; Kwon, M, H. and Shemin, R. J. 2017. “Tricuspid Valve Regurgitation after Heart Transplantation,” Armals of Cardiothoracic Surgery (Hong Kong, China) 6(3): 270-274; Goekler, J., Zuckermann, A., Osorio, E., Brkic, F. F., Uyanik-Uenal, K., Laufer, G., and Aliabadi-Zuckermann, A. 2017. “Cardiac Surgery after Heart Transplantation: Elective Operation or Last Exit Strategy?,” Transplantation Direct 3(10): e209; Jacob, S. and Sellke, F. 2009. “Is Bicaval Orthotopic Heart Transplantation Superior to the Biatrial Technique?,” Interactive Cardiovascular and Thoracic Surgery 9(2): 333-342; Wong, R. C., Abrahams, Z., Hanna, M., Pangrace, J., Gonzalez-Stawinski, G., Starling, R., and Taylor, D. 2008. “Tricuspid Regurgitation after Cardiac Transplantation: An Old Problem Revisited,” Journal of Heart and Lung Transplantation (New York, New York) 27(3): 247-252).

Transplantation thus-without entry into either heart, zero ischemia, continuous, uninterrupted flow of normothermal pulsatile blood, without cardiopulmonary support and the risk of postperfusion syndrome with its cytokine induced inflammation, or a postoperative loss in cognitive acuity, and no cross-clamping-cannot be accomplished without vascular valves. The recipient heart pending excision, a residual ill effect of obtaining increased propulsive force through the use of a high dose of inotropic drugs such as dopamine or adrenaline may be discounted.

Unless the positioning of donor and recipient prompt the use of connecting lines longer than usual, mainlines with build in in-line booster pumps should not be necessary. Whereas here the hearts of both donor and recipient supply propulsive force, the need therefor by Lillehei and coworkers responded to the heart of an adult called upon to supply blood for the small patient as well as himself. Such a jumper can incorporate an additional length of line for removal once the graft organ has been transplanted or a booster pump whether the additional line length made such necessary, or both.

Sudden and metered compound vascular bypass, or sudden switched-organ transplantation using the unconventional total orthotopic technique described herein rather than the bicaval technique omits incision into and anastomosis of the atria a deterrent to solid organ transplantation in the fetus due to the minuteness of the stitches required. Moreover, use of the expansion fabric offers greater latitude in the exact positioning of the new heart within the chest, and eliminates the need for lengthy microsurgery, with the valve jacket accessory channels allowing drugs to be targeted directly into the jackets and lines.

Additional adaptability in size can also be obtained with tubing made of a shrinkable polymer with a wall thickness and heat transmissibility that will protect the blood coursing through the tubing from damage. To minimize stiffness, the material must be highly plasticized with any plasticizer residue completely removed. If necessary to protect underlying tissue, a heat shield is placed behind the tubing during shrinking. Biocompatible shrinkable tubing materials include polytetrafluoroethylene; fluorinated ethylene propylene (hexafluoropropylene and tetrafluoroethylene copolymer); perfluoroalkoxy alkane (tetrafluoroethylene and perfluoroether copolymer); ethylene tetrafluoroethylene (poly(ethene-co-tetrafluoroethene); and clear transparent polypropylene or polyethylene terephthalate (poly(ethylene terephthalate).

Newer materials shrinkable polyether ether ketone; and polytetrafluoroethylene/fluorinated ethylene propylene bilayer heat shrink developed by Zeus Industrial Products Corporation, Orangeburg, South Carolina. The ratio of unstretched-to-stretched length is used to cut each line to length so that the line lumen will be substantially cylindrical at the final length determined by the positioning of the graft in the recipient. The resultant even and anticoagulant internal surface of the tubing minimize if not eliminate the need for a slow heparin such as enoxaparin sodium drip to prevent a buildup of clot.

Suture lines often initiate discharges that trigger a dysrhythmia. Here there are no suture lines, and an implantable cardioverter-defibrillator can be placed to counter a timing problem of alternative origin. In double heart arrangements as will be addressed, implantable cardioverter-defibrillators or pacemaker/cardioverter-defibrillators, while likely to prove removable after the hearts have adapted to their new milieu are best placed in both hearts. Nontraumatizing methods for removing transvenous leads are addressed below in section 2b(1)(b)(b5), entitled Heterotopic Double Heart Metered Compound Vascular Bypass, or Sudden Switched-organ Transplantation [and so on].

This is because:
1. The foam lining of the jackets is sufficiently thick to allow much growth.
2. The spring hinges expand with increased diameter in the substrate ductus to hold the jackets snugly in place without locking.
3. The mainlines conveying blood and accessory channels or sidelines targeting drugs directly to the jacket and lines comprise extendable coiled small-caliber tubes or catheters with restorative force sufficient to find the least length without exerting tension at the attachment at either end, such tubing assuring consistency rather than a reduction in the diameter of the tubing when elongated, and
4. To facilitate the reassignment of lines to valves at different locations, those electrical can be eliminated with ‘lines’ radio controlled, such as Bluetooth and power delivered through transcutaneous energy transfer. If used, wire is coiled for extension and routed to avoid strangulation of intervening tissue.

If necessary, resistance to extension is reported by implant sensors (see, for example, Chiriacò, M. S., Bianco, M., Nigro, A., Primiceri, E., Ferrara, F., and 5 others 2018. “Lab-on-Chip for Exosomes and Microvesicles Detection and Characterization,” Sensors (Basel, Switzerland) 18(10). pii: E3175; Amjadi, M., Pichitpajongkit, A., Lee, S., Ryu, S., and Park, I. 2014. “Highly Stretchable and Sensitive Strain Sensor Based on Silver Nanowire-Elastomer Nanocomposite,” American Chemical Society Nano 8(5): 5154-5163. Depending upon their detailed chemistry and freedom from residual plasticizer, biocompatible elastic convoluted and accordion-configured tubing are likewise applicable, and 4. Intracorporeal electrical connections are not hard wired but rather control commands delivered by wireless means through Bluetooth radio communication to the master node control microprocessor, with power delivered through transcutaneous energy transfer.

To eliminate pressures that would result in hemolysis, relative consistency rather than a reduction in the diameter of the tubing when elongated is essential (see, for example, von Segesser, L. K. 1999, Op cit.). In an adult sudden or metered switch procedure where no need exists to accommodate growth, the mainlines connecting the diversion jackets encircling the corresponding vessels of donor and recipient are shortened by cutting out the excess length of tubing.

To provide the excess length of tubing, that is, the additional length needed to reach from the recipient to the donor and not interfere with the surgical team, mainlines for sudden or metered compound vascular bypass, or sudden switched-organ transplantation are made with a diversion bypass path longer than the distance between the opposing free ends of the centrally transected mainline to either side were these placed in flush continuous relation.

The diversion jackets, both on the mainline, one just short of the retrograde end of the bypass to redirect blood from the mainline into the bypass and one just short of the upstream or entry end of the bypass to redirect blood back into the mainline, have chutes fully extended to pass blood moving through the mainlines without obstruction through the bypass from one jacket to the other.

Until the transplant organ is positioned in the recipient, the chutes remain fully extended, with flow through the bypass. Once the transplant organ is positioned in the recipient, the free ends of the mainline are brought into flush relation for continuous flow, the junction wrapped about by a thin plastic band bonded about the mainlines with surgical cyanoacrylate cement, in turn wrapped about with a thin nonallergenic plastic surgical tape. The chutes are then fully retracted to pass blood directly through the mainline past the closed off bypass.

The jackets with connecting bypass line can then be removed, or if a direct path to them through the surface port is desired, or if follow-up control over the flow volume through the mainline is desired, either or both jacket mainlines are left in place with accessory channels to the surface port left intact. Since exceptionally, the diversion jackets are temporarily positioned along a synthetic rather than implanted along a native conduit, intravenous therapy Y-sets, referred to as ‘piggyback’ catheters, can be adapted for the purpose; however, this requires two at both inflowing and outflowing ends of the mainline where the bypass is connected.

While donor and recipient are beside one another, the piggybacks along the mainline are closed and those at the ends of the bypass opened. When the transplant organ is placed in the recipient, these settings are reversed, so that flow is directly through the mainline past the bypass, which can then be removed. Such an arrangement does not afford accessory channels for the direct delivery of drugs into the mainlines from the surface port or, where mainlines larger in luminal diameter than those native have been used intentionally, control over the degree of extension into the lumen of the chutes to modulate blood flow when appropriate once the recipient is closed.

Other such segment removal arrangement likewise do not afford these capabilities. Thus, alternatively, where the retention of jackets for their accessory channels is not sought and the added expense of bypass jackets is to be avoided, the supplier furnishes a jacketless full-length mainline transected at the center with a bypass arching over from one to the other free end of the mainline, typical fixed lengths for the arching bypass section generally ranging up to 2 feet. Toward either end of the mainline section to either side is a tiny drop gate that in the blocking position causes blood to flow through the bypass. A second pair of drop gates or sliding covers surround the junction of the bypass at either end where it is continuous with the mainline, each to its respective side.

When the transplant organ has been positioned in the recipient, the facing ends of the mainline to either side are bonded together, the drop gates at the junction of the bypass with the mainline to either side closed, and the drop gates along the mainline opened, thus allowing blood to flow through the shortened mainline. With a young patient, the coiled plastic extension spring urges the lines to their least freely reached length but is able to lengthen considerably when its attachments recede from one another (see Reum, D. J. 1978. Method for Producing a Spirally Wound Plastic Article, U.S. Pat. No. 4,120,929).

This arrangement allows growth for a period of years, allowing the patient to fully stabilize and adapt to the new heart without surgical trauma. Eventual removal and replacement of the lines in fetuses and neonates is not required to accommodate growth. A plastic rather than metal coil spring is used as much less thrombophilic, and if and only if necessary, clotting can be prevented by a slow low molecular weight or enoxaparin sodium heparin drip initiated at the surface port targeted into the lines through the accessory channels.

The stock of the helical plastic spring is round, not flat. Such coiled lines serve not only to accommodate considerable growth but considerably reduce the length of the lines during the sudden or metered compound vascular bypass, or sudden switched-organ transplantation procedure described in the next section when donor and recipient must be separated by a distance sufficient to allow members of the surgical team to move between the two. Provided the outer surface of the plastic film coil springs is given a lubricious coating, the risk of a line strangulating an intervening organ or tissue or catching onto or abrading neighboring tissue when contracting following organ placement is virtually nil.

All surfaces of the mainline spring-plastic tube are coated with the lubricious coating—the round plastic spring stock to further avert the formation of clot, and both inner and outer surfaces of the plastic sheath, allowing free movement past the outer edges of the spring turns within and neighboring tissue without. The moderate restorative force of the spring urges it to its shortest length without placing undue tension on its attachments, and the slippery surfaces of the spring turns and internal and external surfaces of its sheath keep it from failing to achieve this smallest length by slipping past neighboring tissue.

The hermetic, such as chemical vapor deposited (Parylene® (Diamond-MT [owner initials]. Johnstown, Pennsylvania) C or N), coating or a similar coating, which completely biocompatible, is used to coat numerous implants such as stents (see, for example, Schwarz, J. A., Contescu, C. I., and Putyera, K. 2004. Dekker Encyclopedia of Nanoscience and Nanotechnology, Volume 1. page 263. Boca Raton, Florida: Chemical Rubber Company Press), inhibits the deposition of clot, and prevents the catching onto or abrasion of neighboring tissue, as well as prevents fluid infiltration. In the mainlines, this prevents extravasation, and in the sidelines, fluid infiltration or leakage, assuring that dosing is accurate. Such a physical vapor deposited coating can be provided by several companies to include Acree Technologies Incorporated, Concord, California, Para-Coat Technologies, Johnstown, Pennsylvania, and KDF Electronic and Vacuum Services Incorporated, Rockleigh, New Jersey.

In contrast, the jackets and bypass lines used in solid organ transplants using the means described herein in an adult can remain in place indefinitely, eliminating the need to reenter. These factors simplify and expedite replacement rather than inadequate palliative surgery after birth and encourage the transition from transluminal valvuloplasty and the balloon dilatation of great vessels to organ replacement in the fetus. Side-entry diversion jackets make possible the in-utero transplantation of solid organs to include the heart, thereby rectifying multiple cognitive, digestive, and other deficits that would result with any other treatment, pre- or post-partum.

Prior to its excision, the native heart unable to generate sufficient propulsive force to move blood through the connecting lines to the donor is provided with percutaneous, or transcutaneous, left or biventricular assistance (see, for example, Garzon-Rodriguez, J. D., Obando-Lopez, C., Giraldo-Grueso, M., Sandoval-Reyes, N., Camacho, J., and Umana, J. P. 2018. “Mechanical Circulatory Support as Bridge Therapy for Heart Transplant: Case Series Report,” Online, BioMed Central Research Notes 11(1): 430; den Uil, C. A., Akin, S., Jewbali, L. S., Dos Reis Miranda, D., Brugts. J. J., and 3 others 2017. “Short-term Mechanical Circulatory Support as a Bridge to Durable Left Ventricular Assist Device Implantation in Refractory Cardiogenic Shock: A Systematic Review and Meta-analysis,” European Journal of Cardiothoracic Surgery 52(1): 14-25; Takeda, K., Garan, A. R., Ando. M., Han, J., Topkara, V. K., and 6 others 2017. “Minimally Invasive CentriMag Ventricular Assist Device Support Integrated with Extracorporeal Membrane Oxygenation in Cardiogenic Shock Patients: A Comparison with Conventional CentriMag biventricular Support Configuration,” European Journal of Cardiothoracic Surgery 52(6): 1055-1061; Mandawat, A. and Rao, S. V. 2017. “Percutaneous Mechanical Circulatory Support Devices in Cardiogenic Shock,” Circulation. Cardiovascular Interventions 10(5). pii: e004337; Miller, P. E., Solomon, M. A., and McAreavey, D. 2017. “Advanced Percutaneous Mechanical Circulatory Support Devices for Cardiogenic Shock,” Critical Care Medicine 45(11): 1922-1929; Kuchibhotla, S., Esposito, M, L., Breton, C., Pedicini. R., Mullin, A., and 8 others 2017. “Acute Biventricular Mechanical Circulatory Support for Cardiogenic Shock,” Journal of the American Heart Association (Oxford, England) 6(10). pii: e006670; Borisenko, O., Wylie, G., Payne, J., Bjessmo, S., Smith, J., Yonan, N., and Firmin, R. 2014. “Thoratec CentriMag for Temporary Treatment of Refractory Cardiogenic Shock or Severe Cardiopulmonary Insufficiency: A Systematic Literature Review and Meta-analysis of Observational Studies” American Society for Artificial Internal Organs Journal 60(5): 487-497; Vollroth, M., Barten, M. J., Mohr, F. W., and Garbade, J. 2012. “Biventricular Levitronix CentriMag Assist Device: A “Bridge to Recovery” Solution in Patients with Acute Fulminant Myocarditis,” Case Reports in Surgery 2012:907490; Loforte, A., Montalto, A., Ranocchi, F., Della Monica, P, L., Casali, G., Lappa, A., Contento, C., Musumeci, F. 2011. “Levitronix CentriMag Third-generation Magnetically Levitated Continuous Flow Pump as Bridge to Solution,” American Society for Artificial Internal Organs Journal 57(4): 247-253; Shuhaiber, J. H., Jenkins, D., Berman, M., Parameshwar, J., Dhital, K., Tsui. S., and Large, S, R. 2008. “The Papworth Experience with the Levitronix CentriMag Ventricular Assist Device,” Journal of Heart and Lung Transplantation (New York, New York) 27(2): 158-164.

While continuous flow impeller or rotor driven assist devices cause problem bleeding due to injury of the von Willebrand factor and possibly damage to thrombocytes, as well as cognitive impairment and breathing difficulty due to capillary damage, supportive use during a switch transplantation is too short in duration to result in such sequelae. Damage to blood components and thrombophylic synthetic materials that cause the accumulation of clot are characteristic of machine means for circulatory support when continued over a longer period.

While in the clinic, blood replacement and the closely monitored administration of an anticoagulant decrease if not eliminate such problems, long-term use by patients who cannot be relied upon for compliance often leads to problems that an organ transplant supported by an implanted response system with sensors and automatic drug release avoids. These and other adverse consequences of continuous flow ventricular assist devices is addressed below in section 2b(1)(b)(b5) entitled Heterotopic Double Heart Compound Vascular Bypass, or sudden switched-organ Transplantation [and so on], wherein the preferability of the measures described herein over alternative approaches is explained.

When the valves are switched to bypass the native heart, pressurized blood neither flows into or out of it, although the cardiac vessels must be clamped to prevent the blood within the heart from running out. When removed, the blood in the donor heart replaces that in the native heart when excised. As addressed below, once the donor heart has been removed, other organs to be harvested are sustained by an artificial heart or sealing off the cardiac vascular stumps and continuing life support by extracorporeal membrane oxygenation, administering replacement hormones, keeping the body warm, and so on. If applicable, donor sustainment in response to a catecholamine storm, vasoplegia, and hemodynamic impairment is conventional (see, for example, Shetty, V, L., Mali, S. S., Shetty, S. V., and Shinde, P, D. 2017, Op cit.).

In conventional total orthotopic heart transplantation, the heart is removed from the donor in full with relatively short vascular stumps; here, total orthotopic transplantation pertains no less to the recipient, who does not retain a portion of the posterior wall of the heart. Additionally, the vascular stumps are at least long enough to accommodate the valving. The evidence shows that the more the surgery recedes from the heart proper so that donor heart remains intact, the less plagued by complications will be the outcome. The pattern of reduced sequelae with increasing withdrawal is seen moving from biatrial to bicaval to conventional donor-only total orthotopic removal, and is anticipated to be further borne out with recipient—also total removal. That is, further receding from its center of mass in addition to not entering the heart results in progressively fewer adverse sequelae.

Conventional total orthotopic heart transplantation where the recipient atria are left and only the donor heart anterior thereto removed in its entirety tends to produce outcomes superior to either biatrial or bicaval transplantation (see, for example, Jungschleger, J, G. M., Boldyrev. S. Y., Kaleda, V. I., and Dark, J, H. 2018. “Standard Orthotopic Heart Transplantation,” Annals of Cardiothoracic Surgery (Hong Kong, China) 7(1): 169-171; Rivinius, R., Helmschrott, M., Ruhparwar, A., Erbel, C., Gleissner, C. A., and 5 others 2017, Op cit.; Toscano, G., Bottio, T., Gambino, A., Bagozzi, L., Guariento, A., and 4 others 2015. “Orthotopic Heart Transplantation: The Bicaval Technique,” Multimedia Manual of Cardiothoracic Surgery 2015. pii: mmv035; Cheng, A. and Slaughter, M. S. 2014. “Heart Transplantation,” Journal of Thoracic Disease 6(8): 1105-1109; Kara, I., Ay, Y., Yanartas, M., Koksal, C., Toker, M. E., Yildirim, T., and Balkanay, M. 2012. “Does the Surgical Technique Used in the Orthotopic Heart Transplant Affect the Results Regarding the Rhythm?,” Anadolu Kardiyoloji Dergisi (Anatolian Journal of Cardiology) 12(3): 255-260; Locali, R. F., Matsuoka, P. K., Cherbo, T., Gabriel, E. A., and Buffolo, E. 2010. “Should Biatrial Heart Transplantation Still be Performed?: A Meta-Analysis,” Arquivos Brasileiros de Cardiologia (Brazilian Archives of Cardiology, Sao Paulo, Brazil) 94(6): 829-840; Schnoor, M., Schafer, T., Luhmann, D., and Sievers, H, H. 2007. “Bicaval Versus Standard Technique in Orthotopic Heart Transplantation: A Systematic Review and Meta-analysis,” Journal of Thoracic and Cardiovascular Surgery 134(5): 1322-1331; Morgan, J, A. and Edwards, N. M. 2005. “Orthotopic Cardiac Transplantation: Comparison of Outcome Using Biatrial, Bicaval, and Total Techniques,” Journal of Cardiac Surgery 20(1): 102-106).

The bicaval technique, does however, avoid right atrial anastomosis—a deterrent to transplantation in a prenate, neonate, or infant due to the minuteness of the stitches required; reduces the incidence of tricuspid valve insufficiency, in eliminating the left as well the right atrial anastomosis, is associated with the lowest rate of post-transplantation arrhythmia; dysrhythmias controllable by implanting a pacemaker, offers somewhat greater latitude in the exact positioning of the new heart within the chest of the recipient; reduces procedural and anesthetic time; reduces trauma, extending applicability to debilitated patients; and eliminates the need for lengthy biatrial microsurgery anastomosis not essential to assure normal growth.

With minimal trauma to both recipient and graft organ, the ability of both patient and heart to tolerate post-transplantation heart surgery is improved (see, for example, Goekler, J., Zuckermann, A., Osorio, E., Brkic, F. F., Uyanik-Uenal, K., Laufer, G., and Aliabadi-Zuckermann, A. 2017, Op cit. above in this section; Holmes, T. R., Jansz, P. C., Spratt, P., Macdonald, P. S., Dhital, K., and 5 others 2014. “Cardiac Surgery is Successful in Heart Transplant Recipients,” Heart, Lung. and Circulation 23(8): 703-710; Coskun, K. O., Coskun, S. T., El Arousy, M., Amin Parsa, M., and 5 others 2007. “Cardiac Surgery after Heart Transplantation: Coronary Artery Bypass Grafting and Heart Valve Replacement,” Heart Surgery Forum 10(2): E110-E114; additional references cited above in section 2a(3), entitled Remotely Procured Graft Organ Preservation).

Because in a sudden compound vascular bypass, or sudden switched-organ transplantation the intravascular diversion valves needed to connect donor and recipient vessels must encircle tubular structures, what begins as the conventional total orthotopic technique must be modified. Specifically, connection of the donor to the recipient organ cannot be withdrawn to just the boundary of the transplant organ but must be shifted further cranially and caudally to include the inflow and outflow vessels. Essentially, the transition from biatrial to bicaval technique demonstrates that the more intact, and the more distant are the incisions from, the donor organ, the more likely will the outcome prove favorable. As a consequence, more tissue is harvested with less substantive dissection.

A solid organ transplant in which the circulation of the recipient is switched from his own diseased organ to that of the donor through a compound vascular bypass between the same supply and drainage vessels is therefore by attachment to the vessels of the organ and entirely outside of and noninvasive of the organ itself. A heart transplant, for example, is extracardiac, hence, closed-heart, eliminating the risks and complications of intracardiac surgery. Provided rejection can be suppressed, that heterotopic transplantation can allow two substandard hearts to approach the function of one good heart eases the shortage of good replacement hearts, especially for neonates.

The ability of the control system to directly target supportive medication to a transplant organ makes possible an expansion in the zone of extended criteria donor, or less than ideal replacement organs available. In an orthotopic heart transplant using the switch technique, the native heart, to include the atria, is completely removed and replaced with an intact transplant heart which must be competent to stand on its own. In a conventional heterotopic, or ‘piggyback,’ heart transplant, the left atria of the native heart are left in the recipient, then joined, or anastomosed to those of the transplanted “backup” heart, which then replaces the incompetent ventricles of the native heart.

The assist heart is entered at four points, inflicting much trauma on both hearts. Double heart switch transplantation to add a second, competent heart to that native, or to replace the native heart with two less than ideal hearts which working together can approximate the function of a competent heart is addressed below in section 2b(1)(b)(b5), entitled Heterotopic Double Heart Compound Vascular Bypass, or Sudden Switched-organ Transplantation [and so on]. Sufficiency in heart and lung function reciprocal, unless the product of short-term pulmonary disease, transplantable hearts are associated with transplantable lungs.

However, severe and/or long-term pulmonary disease damages the heart, so that in a patient with heart and lung disease able to withstand the surgery, a heart and lung transplant should be performed. Where the death of a healthy donor resulted from accidental trauma that left the heart and lungs intact, both will be transplantable. The connections required for sudden or metered compound vascular bypass, or sudden switched-organ transplantation the same using either, in a heart transplant, pulmonary and systemic arterial and venous flow is diverted from the diseased donor heart to and through the heart of the donor.

To transplant the lungs, only the pulmonary arteries and veins of the recipient are diverted to and through the lungs of the donor, who if to be preserved for further organ donation, is sustained on extracorporeal membrane oxygenation. Harvesting the heart along with the lungs then, is by specifically omitting the pulmonary arteries and veins, effectively replacing the systemic circulatory system of the recipient with that of the donor. With sudden and metered compound vascular bypass, or sudden switched-organ transplantation, removal of the heart and lungs from the recipient is accomplished only after blood flow has been switched to the donor. The supply and drainage vasculature clamped prior to removal, bleeding due to causes other than the dissection of pleural adhesions is substantially reduced.

These procedures also simplify and reduce the procedural time of a heart-lung transplant (see, for example, Blitz A. 2017. “Surgical Techniques of Heart Transplantation and Heart-Lung Transplantation, in Subramaniam, K., Sakai T., eds., Anesthesia and Perioperative Care for Organ Transplantation, Springer, New York, New York; Yun, J. K., Choi, S. H., and Park, S. I. 2016. “Clinical Outcomes of Heart-Lung Transplantation: Review of 10 Single-center Consecutive Patients,” Korean Journal of Thoracic and Cardiovascular Surgery 49(3): 157-164; Huddleston, C. B. and Richey, S, R. 2014, Op cit.; Hirche, T. O., Knoop, C., Hebestreit, H., Shimmin, D., Sole, A., and 5 others 2014. “Practical Guidelines: Lung Transplantation in Patients with Cystic Fibrosis,” Pulmonary Medicine 2014:621342; Panchabhai, T. S., Chaddha, U., McCurry, K. R., Bremner, R. M., and Mehta, A, C. 2018. “Historical Perspectives of Lung Transplantation: Connecting the Dots,” Journal of Thoracic Disease 10(7): 4516-4531).

Moreover, where disease has affected the airway of the recipient, the donor trachea can be transected closer to the cricoid cartilage than at the mainstem bronchus level, as it is in the conventional Metras-Pasque procedure, thus transferring more of a competent airway with the lungs (Panchabhai, T. S., Chaddha, U., McCurry, K. R., Bremner, R. M., and Mehta, A, C. 2018, Op cit.; Gust, L., D'Journo, X. B., Brioude, G., Trousse, D., Dizier, S., and 3 others 2018. “Single-lung and Double-lung Transplantation: Technique and Tips,” Journal of Thoracic Disease 10(4): 2508-2518; Venuta, F. and Van Raemdonck, D. 2017. “History of Lung Transplantation,” Journal of Thoracic Disease 9(12): 5458-5471; Dabak, G. and Şenbaklavaci, O. 2016. “History of Lung Transplantation,” Turkish Thoracic Journal 17(2): 71-75; Yeung, J, C. and Keshavjee, S. 2014. “Overview of Clinical Lung Transplantation,” Cold Spring Harbor Perspectives in Medicine 4(1): a015628; Bolman, R. M. 3rd, Shumway, S., Estrin, J. A., and Hertz, M. I. 1991. “Lung and Heart-Lung Transplantation. Evolution and New Applications,” Annals of Surgery (Philadelphia, Pennsylvania) 214(4): 456-470; Kaiser, L. R., Pasque, M. K., Trulock, E. P., Low, D. E., Dresler, C. M., Cooper, J, D. 1991. “Bilateral Sequential Lung Transplantation: The Procedure of Choice for Double-lung Replacement,” Annals of Thoracic Surgery 52(3): 438-446; Pasque, M. K., Cooper, J. D., Kaiser, L. R., Haydock, D. A., Triantafillou, A., and Trulock, E. P. 1990. “Improved Technique for Bilateral Lung Transplantation: Rationale and Initial Clinical Experience,” Annals of Thoracic Surgery 49(5): 785-791: Cooper, J, D. 1990. “The Evolution of Techniques and Indications for Lung Transplantation,” Annals of Surgery (Philadelphia, Pennsylvania) 212(3): 249-256). Lung transplantation is addressed in passing below in section 2b(1)(b)(b2), entitled Prenatal Compound Vascular Bypass, or Sudden Switched-organ Transplantation of the Heart.

By the same token, if the upper trachea is affected, transection and anastomosis can be conventional, namely, at the mainstem bronchus level. In these and other solid organ transplantation procedures, the diseased organ is isolated from the circulation, but at no time are the graft organ or organs disconnected from a normal blood supply and drainage. Rather, these are switched from the recipient to that of the donor-suddenly with the sudden, and gradually with the metered switch technique. A significant reduction in bleeding is also a central advantage of switch transplantation in in liver transplantation, where problem bleeding has been problematic. Additionally, where vascular anastomoses are anticipated to be a source of failure, the procedure allows anastomoses and the discharges from the suture lines that induce dysrhythmias to be avoided, either or both by avoiding direct end-to-end anastomoses or releasing an anesthetic on the anastomosis.

In heart transplantation, transection of the atria as performed with the biatrial technique opens the way for atrial enlargement with dysrhythmias, arrhythmias, atrioventricular block, and left bundle branch block, frequency conduction problems, valve incompetence, and impaired filling of the left ventricle, hence, the need at least temporarily for a pacemaker (see, for example, Wdowczyk, J., Makowiec, D., Gruchała. M., Wejer, D., and Struzik, Z, R. 2018. “Dynamical Landscape of Heart Rhythm in Long-term Heart Transplant Recipients: A Way to Discern Erratic Rhythms,” Online, Frontiers in Physiology 9:274. Op cit. above in section 2. Background of the Invention. a. General: Bulut, M., Evlice, M., Celik, M., Eren, H., Savluk, O. F., and 7 others 2017. “Atrial Electromechanical Delay in Patients Undergoing Heart Transplantation,” Journal of Arrhythmia 33(2): 122-126; Mallidi. H, R. and Bates, M. 2017. “Pacemaker Use Following Heart Transplantation,” Ochsner Journal 17(1): 20-24; AlTurki, A., Proietti, R., Bimie, D. H., and Essebag. V. 2016. “Management of Antithrombotic Therapy during Cardiac Implantable Device Surgery,” Journal of Arrhythmia 32(3): 163-169; Cheng, A. and Slaughter, M. S. 2014, Op cit.; Thajudeen, A., Stecker, E. C., Shehata, M., Patel, J., Wang, X., and 3 others 2012. “Arrhythmias after Heart Transplantation: Mechanisms and Management,” Journal of the American Heart Association (Oxford, England) 1(2): e001461, also cited above in section 2. Background of the Invention. a. General: Mehrzad, R., Rajab, M., and Spodick, D, H. 2014. “The Three Integrated Phases of Left Atrial Macrophysiology and Their Interactions,” Online, MDPI [Multidisciplinary Digital Publishing Institute] International Journal of Molecular Sciences (Basel, Switzerland) 15(9): 15146-15160; Boudoulas, K. D., Paraskevaidis, I. A., Boudoulas, H., and Triposkiadis, F. K. 2014. “The Left Atrium: From the Research Laboratory to the Clinic,” Cardiology 129(1): 1-17; Dahu, M. I. and Hutchinson, M, D. 2012. “What is the Mechanism of the Atrial Arrhythmia in a Patient after Orthotopic Heart Transplantation?,” Journal of Cardiovascular Electrophysiology 23(2): 225-227; Markowicz Pawlus, E., Duszańska, A., Przybylski, R., Szulik, M., Streb. W., and 3 others 2012. “Does the Method of Heart Transplantation Affect Left Ventricular Filling?,” Kardiologia Polska 70(8): 769-773; Kara, I., Ay, Y., Yanartaş, M., Koksal. C., Toker, M. E., Yildirim, T., and Balkanay, M. 2012. “Does the Surgical Technique Used in the Orthotopic Heart Transplant Affect the Results Regarding the Rhythm?,” Anadolu Kardiyoloji Dergisi (Anatolian Journal of Cardiology) 12(3): 255-260; Gutierrez, C. and Blanchard, D, G. 2011. “Atrial Fibrillation: Diagnosis and Treatment,” American Family Physician 83(1): 61-68; Meyer, S. R., Modry, D, L., Bainey, K., Koshal, A., Mullen, J. C., and 4 others 2005. “Declining Need for Permanent Pacemaker Insertion with the Bicaval Technique of Orthotopic Heart Transplantation,” Canadian Journal of Cardiology 21(2): 159-163; Traversi, E., Pozzoli, M., Grande, A., Forni, G., Assandri, J., Viganò, M., and Tavazzi, L. 1998. “The Bicaval Anastomosis Technique for Orthotopic Heart Transplantation Yields Better Atrial Function than the Standard Technique: An Echocardiographic Automatic Boundary Detection Study,” Journal of Heart and Lung Transplantation (New York, New York) 17(11): 1065-1074).

Rather, the mainlines need not be disconnected for a time if ever but allowed to remain connected to the implant, with the vascular stumps end-to-end anastomosed, as addressed below in the section entitled 1. Background of the Invention. a. General. Once the graft organ has been harvested, it is also possible to cross-clamp the stumps or directly anastomose the stumps of supply arteries to downstream arteries. If there is concern that small caliber vessel anastomoses will hold or emit discharges, connection can be through valve-jackets or the small vessels can be clamped upstream at the larger supply artery. Another pattern of improvement obtained moving from bicaval to donor total heart transplantation, thence to donor and recipient total transplantation is gaining latitude in the exact positioning of the heart in the chest of the recipient.

Treatment of the stumps in the donor is different depending upon whether the donor is to continue as a source for other organs. When no longer needed, the stumps of the donor can be clamped shut and life support removed. When the donor having been continued on life support past death is to continue as a source of organs, the donor jackets are straddled, or bridged over, by upstream and downstream stump jackets that will bypass the graft organ at the same instant it is fully transferred.

In metered compound vascular bypass, or sudden switched-organ transplantation, unlike the linear servovalve-controlled valves used to gradually transfer the graft organ from the circulatory system of the donor into the circulatory system of the recipient or to do so intermittently in increments, the straddling jackets can be solenoid controlled (see, for example, Chong Li, C., Yang, H., Jenkins, L. L, and Dean, R. N., Flowers, G. T., Hung, J. Y. 2016. “Enhanced-Performance Control of an Electromagnetic Solenoid System Using a Digital Controller,” IEEE [Institute of Electrical and Electronics Engineers] Transactions on Control Systems Technology (New York, New York) 24(5): 1805-1811). Such eliminates both the need for clamping, and taking effect at the same time as the graft organ has been transferred, the loss of blood. Where the loss of blood while the stumps are manually clamped is not a concern, straddling or outrigger jackets can be omitted.

While rare, additional flexibility in positioning is especially advantageous in replacing the heart of a neonate with dextrocardia and severe cardiac malformity. Ischemic time eliminated in sudden compound vascular bypass, or sudden switched-organ transplantation, that use of the Shumway-Lower-Stofer, or biatrial, technique reduces ischemic time compared to alternative methods is immaterial. Any atherosclerotic, vasculitic, or connective tissue disorder of these vessels in the recipient is replaced along with the diseased or malformed heart, and there is no duplication of the sinoatrial nodes. In an adult with diseased vasculature, the supply and drainage vessels can be taken with the organ proper, or the synthetic mainlines with accessory channels can be left as a prosthesis, the stumps if not anastomosed tied off.

Where the immunological milieu had caused the injury to the native organ, as the gut in inflammatory bowel syndrome, transplantation of the graft organ with vessels or the replacement of these with genetically true tissue engineered vessels will expose these along with the organ proper to the same conditions, promoting recrudescence in the transplants or engineered grafts. Where this is the case, the use of synthetic materials, oblivious to aberrations of biology, are to be preferred. Furthermore, synthetic lines as vessels are connected with side-entry jackets, valve-jackets, or nonjacketing side-entry connectors, all of which incorporate one or more accessory channels piped to a small port at the body surface which allow immunosuppressant drugs to be directly pipeline targeted into the catheteric lines used as vessels.

Tissue engineered organs, glands, and tissues autologously, or autogeneically, true to the patient will not be oblivious to the autoimmunological milieu. Such grafts will replace segments of small bowel removed to alleviate refractory Crohn's disease in adults and necrotizing enterocolitis in the young, for example, where such removal had resulted in short bowel syndrome (see, for example, Martin, L. Y., Ladd, M. R., Werts, A., Sodhi, C. P., March, J. C., and Hackam, D. J. 2018. “Tissue Engineering for the Treatment of Short Bowel Syndrome in Children,” Pediatric Research 83(1-2): 249-257; Cromeens, B. P., Wang, Y., Liu, Y., Johnson, J., and Besner, G, E. 2018. “Critical Intestinal Cells Originate from the Host in Enteroid-derived Tissue-engineered Intestine,” Journal of Surgical Research 223:155-164; Ladd, M. R., Niño, D. F., March, J. C., Sodhi, C. P., and Hackam, D. J. 2016. “Generation of an Artificial Intestine for the Management of Short Bowel Syndrome,” Current Opinion in Organ Transplantation 21(2): 178-185; Belchior, G. G., Sogayar, M. C., and Grikscheit, T, C. 2014. “Stem Cells and Biopharmaceuticals: Vital Roles in the Growth of Tissue- engineered Small Intestine,” Seminars in Pediatric Surgery 23(3): 141-149; Spurrier, R. G., Speer, A, L., Grant, C. N., Levin, D. E., and Grikscheit, T, C. 2014. “Vitrification Preserves Murine and Human Donor Cells for Generation of Tissue-engineered Intestine,” Journal of Surgical Research 190(2): 399-406; Spurrier, R, G. and Grikscheit, T, C. 2013. “Tissue Engineering the Small Intestine,” Clinical Gastroenterology and Hepatology 11(4): 354-358; Levin, D. E., Barthel, E. R., Speer, A, L., Sala, F. G., Hou, X., Torashima, Y., and Grikscheit, T, C. 2013. “Human Tissue-engineered Small Intestine Forms from Postnatal Progenitor Cells,” Journal of Pediatric Surgery 48(1): 129-137).

Like allografts, such tissue will require to be isolated for directly pipeline-targeted immunosuppressant treatment. Broadly, where the immunological context is not a problem, a tissue engineered replacement organ, gland, or tissue genetically true to the patient is best as least likely to require inordinate vigilance and support. Where this is not the case, artificial internal organs in development will eventually overcome the problems of immune system destruction and incompatibility. However, these will still induce some adverse tissue response to warrant support by an implanted automatic directly targeted drug release response system.

Accessory channels allow the direct pipeline-targeting of an anticoagulant such as a heparin to prevent the accumulation of clot and an antimicrobial, typically an antibiotic to prevent the formation of biofilm. Such lines can also directly support transplant organs with directly targeted immunosuppressants, sparing the rest of the body from exposure. The lack of directly targeted drug support in small caliber catheteric lines that would readily clog explains the limitation to large caliber vessels such as the aortic arch and femorofemoral shunts of polyethylene terephthalate prostheses (Dacron®, Terylene®, Lavsan®) (see, for example, Grus, T., Lambert, L., Mlcek, M., Chlup, H., Honsova, E., and 3 others 2018. “In Vivo Evaluation of Short-term Performance of New Three-layer Collagen-based Vascular Graft Designed for Low-flow Peripheral Vascular Reconstructions,” Online, Biomed Research International 2018:3519596; De Paulis, R., Scaffa, R., Maselli, D., Salica, A., Bellisario, A., and Weltert, L. 2008, Op cit. above in section 2b(1)(a), entitled Sudden Compound Vascular Bypass, or Sudden Switched-organ Transplantation.

While side-entry devices incorporating accessory channels can be fitted to tubing made of any material, the history of failure associated with smaller gauge or catheteric lines will continue with continued attempts to achieve sustainability in vivo of these without the benefit of such directly targeted support (see, for example, Mi, H. Y., Jing, X., Li, Z. T., Lin, Y. J., Thomson, J. A., and Turng, L. S. 2019. “Fabrication and Modification of Wavy Multicomponent Vascular Grafts with Biomimetic Mechanical Properties, Antithrombogenicity, and Enhanced Endothelial Cell Affinity,” Journal of Biomedical Materials Research. Part B. Applied Biomaterials 107(7): 2397-2408; Yu, E., Mi, H. Y., Zhang, J., Thomson, J. A., and Turng, L. S. 2018. “Development of Biomimetic Thermoplastic Polyurethane/Fibroin Small-diameter Vascular Grafts via a Novel Electrospinning Approach,” Journal of Biomedical Materials Research. Part A. 106(4): 985-996; Yu, E., Zhang, J., Thomson, J. A., and Turng, L. S. 2016. “Fabrication and Characterization of Electrospun Thermoplastic Polyurethane/Fibroin Small-diameter Vascular Grafts for Vascular Tissue Engineering,” International Polymer Processing 1(5): 638-646).

Even if successful, the fact that engineered vessels based upon biological sources are placed to repair a result of disease or defect means that these too should be fitted with the appropriate side-entry devices to allow the targeting of medication to the site (see, for example, Colunga, T. and Dalton, S. 2018. “Building Blood Vessels with Vascular Progenitor Cells,” Trends in Molecular Medicine 24(7): 630-641; Gong, Z. and Niklason, L, E. 2008. “Small-diameter Human Vessel Wall Engineered from Bone Marrow-derived Mesenchymal Stem Cells (hMSCs),” Federation of American Societies for Experimental Biology Journal 22(6): 1635-1648).

If the mainlines are so long as would interfere with orthotopic placement of the new heart, then while the mainlines continue to perfuse the graft organ, the stumps are anastomosed, the valves fully opened to allow free flow of blood through the graft organ, and the mainlines removed. Whereas the vessel transections, hence, the anastomoses, used to remove the diseased native organ are at levels proximal to the organ and inside the valve-jackets, the transections in the donor are outside the jackets.

Removal of the mainlines allows the sidelines, or accessory channel lines, connected to a surface port to remain, giving direct therapeutic and diagnostic access to the treatment site; that is, to allow the direct targeting of drugs to the anastomoses from a small port at the surface of the body at least the arterial jackets with accessory channels or sidelines are left. In a baby, the vessels are anastomosed with a surgical grade cyanoacrylate cement, great care exerted to assure leak-free connection. Where anastomosis is deferred or discounted, the synthetic lines and diversion valve-jackets left in place as a prosthesis, any generation of arrhythmogenic discharges from the cut ends of the stumps is treated by targeting an anesthetic to the cut ends through a port at the body surface as entry into the accessory channel or channels leading to the anastomoses.

While less problematic with the total orthotopic than alternative techniques, this should alleviate stray discharges as the source, albeit infrequently, of post-transplantation atrial fibrillation and stroke, sometimes cited as deterrents to the otherwise preferable total orthotopic technique (see, for example, Chokesuwattanaskul, R., Bathini, T., Thongprayoon, C., Preechawat, S., O'Corragain, O. A., and 3 others 2018. “Atrial Fibrillation following Heart Transplantation: A Systematic Review and Meta-analysis of Observational Studies,” Journal of Evidence-based Medicine 11(4): 261-271; Badin, A., Tyler, J., and Kalbfleisch, S. J. 2017. “Atrial Fibrillation Originating from Recipient Left Atrium after an Orthotopic Heart Transplantation,” HeartRhythm Case Reports 3(12): 604-607; Mallidi, H, R. and Bates, M. 2017, Op cit., this section; Acampa, M., Lazzerini, P. E., Guideri, F., Tassi, R., and Martini, G. 2016. “Ischemic Stroke after Heart Transplantation,” Journal of Stroke 18(2): 157-168; Hamon, D., Taleski, J., Vaseghi, M., Shivkumar, K., and Boyle, N, G. 2014. “Arrhythmias in the Heart Transplant Patient,” Arrhythmia and Electrophysiology Review 3(3): 149-155; Peretto, G., Durante, A., Limite, L. R., and Cianflone, D. 2014. “Postoperative Arrhythmias after Cardiac Surgery: Incidence, Risk Factors, and Therapeutic Management,” Cardiology Research and Practice 2014:615987; Sattiraju, S., Vats, S., Krishnan, B., K., Kim, S., Austin, E., and 4 others 2012. “Operative Technique and Atrial Tachyarrhythmias after Orthotopic Heart Transplantation,” Journal of Atrial Fibrillation 5(4): 690; Dasari, T. W., Pavlovic-Surjancev, B., Patel, N., Williams, A. A., Ezidinma, P., and 3 others 2010. “Incidence, Risk Factors, and Clinical Outcomes of Atrial Fibrillation and Atrial Flutter after Heart Transplantation,” American Journal of Cardiology 106(5): 737-741; Ahmari, S. A., Bunch, T. J., Chandra, A., Chandra, V., Ujino, K., and 7 others 2006. “Prevalence, Pathophysiology, and Clinical Significance of Post-heart Transplant Atrial Fibrillation and Atrial Flutter,” Journal of Heart and Lung Transplantation (New York, New York) 25(1): 53-60).

The same approach can be used when anastomosis is immediate. As shown in FIG. 4A, diversion valve-jackets are placed inside the suture lines on the stumps so that the accessory channels, which course through the underside center of the diversion chute are directed to target the graft organ. The anastomoses are therefore outside the jackets making these inaccessible in the direct sense to medication delivered through the accessory channels. The suture lines are therefore directly targeted not through a diversion valve-jacket but rather through a basic side-entry jacket such as that shown in FIG. 3, used to form a permanent junction with a vessel or other ductus placed upstream where diversion in uninvolved. The donor organ incorporated into the circulatory system of the recipient, the defective innate organ is removed and the donor organ positioned in the chest of the recipient.

The positioning adjacent to an anastomosis of a basic side-entry jacket feeding into a flat reservoir positioned subcutaneously in the pectoral region through a miniature peristaltic pump at the outlet controlled by a microcontroller implant where the reservoir is connected by a catheter to a port at the body surface for replenishment allows the automatically released direct targeting of drugs such as immunosuppressant to the anastomosis. Uptake substantially along the local segment, this spares exposure of the rest of the body to the drug or drugs, thereby avoiding the increased susceptibility to disease that results with a reduction in immune function.

Nevertheless, in most instances, a much reduced background systemic dose is prudent. For maximum effect, jackets are positioned both upstream and downstream to the anastomosis. In a neonate or small child, especially one frail, anastomoses are best direct end-to-end, procedural time and the risk of complications deferred if not eliminated by using a surgical grade cyanoacrylate cement rather than suture. In an adult, the tubing used to connect the vessels if not removed when the graft organ is placed in the recipient provides a gap in lieu of direct contact between donor and recipient endothelia, affording an interval for the immunosuppressants delivered upstream to take effect.

For a heart transplant, the chests are opened and diversion valves placed on the corresponding vessels in recipient and donor, each pair then connected by highly elastic coiled tubing. The length of the connecting tubes is made as short as possible without interfering with free movement of the surgical team. While unlikely, if this results in tubes long enough to create a condition of hypovolemia and pressure loss, then before reciprocal cross-circulation is initiated, saline solution of blood is used to fill the line, and if necessary, the diseased heart provided inotropic support until it is removed and the new heart placed. Once the donor organ has been positioned in the recipient, the tubing assumes its shortest non-pulling length. Taking the cardiac vessels along with the heart allows replacing these along with the heart.

If the cardiac vessels of the recipient are whether due to anatomy of pathology, those of the donor not usable, the coiled lines, which considerably shorten when not stretched, are left connected to the stumps as a prosthetic bypass with accessory channels capable of directly targeting medication to the anastomoses, ductus, lines, and jackets. The diversion valves serving to bypass and give access to medicate each prospective anastomosis, the corresponding vessels can then be anastomosed and the mainline, or blood carrying, tubing removed. Direct pipeline targeting to each vessel and anastomosis is through the sidelines, or accessory channels, by injection into the respective opening in the small port at the body surface.

Thus, if disease free, the vessels are anastomosed, the coiled mainlines removed, and the valve-jackets and accessory channels left to directly target medication from the surface port to the anastomoses, the suture lines in a heart transplant implicated as a source of dysrhythmia-inducing discharges (see, for example, Rivinius, R., Helmschrott, M., Ruhparwar, A., Erbel, C., Gleissner, C. A., and 5 others 2017, Op cit.; Locali, R. F., Matsuoka, P. K., Cherbo, T., Gabriel, E. A., and Buffolo, E. 2010. “Should Biatrial Heart Transplantation Still be Performed?: A Meta-Analysis,” Arquivos Brasileiros de Cardiologia (Brazilian Archives of Cardiology, Sao Paulo, Brazil) 94(6): 829-840).

As addressed below, the nonsparking valves are controlled from the same switch, the wires removed before closing. In FIG. 1, for visual clarity, arterial flow, and in FIG. 2, venous flow, through donor and recipient hearts is summarized in the form of unit conduits. FIG. 1A shows the donor organ on the left and recipient organ on the right before switching of the arterial system of the recipient to the donor organ, while FIG. 1B shows the same after the switch has been made. FIGS. 2A and 2B show the same for the venous system.

The figures indicate the orientation of the vascular valve blood flow diversion chutes while retracted before diversion and the position of each following the advancement of each chute into its respective lumen to divert flow to the donor organ. Passage through the valves gradually curved with no protrusions into the passageway and a lumen consistent with that of the substrate native lumen, redirection of flow through the diversion chutes is free of shearing forces. Also, the views are planar, whereas the valves are rotated about the substrate vessel to obtain the most accommodating position in relation to the surrounding tissue.

Some background on ductus side-entry jackets and diversion valves best precedes resumption of a description of the use of these devices in transplantation. A basic ductus side-entry jacket as shown in FIG. 3 makes possible the substantially leak-free and permanent connection of a synthetic tube, usually a conventional catheter, to a native ductus, that is, a tubular anatomical structure, or ductus, such as an artery or a ureter, so that the native and synthetic or tissue engineered lumina are made continuous. In contrast, a vascular valve is a ductus side-entry jacket modified to incorporate a diversion chute.

An intravascular diversion valve such as that shown in FIG. 4, is a ductus side-entry jacket modified to incorporate a diversion chute, which riding along a track or raceway, allows the chute to be advanced from outside into the substrate native lumen, thereby redirecting the flow of blood or urine out through the valve side-stem, or mainline. Retracted, the substrate lumen is closed off from leaking through the mainline by an ostium obturator, so that flow through the original lumen is unaffected. Additional structural details are provided in FIG. 4B.

When driven by a lightweight microminiature damped and nonsparking plunger solenoid, such a diversion valve is limited to binary, or bistable, action, that is, its chute is slidable entirely into or out of the substrate native lumen. Quick solenoid plunger (thrust rod, slider), or full throw end-to-end action thus is ideal for sudden compound vascular bypass, or sudden switched-organ transplantation, but not where to allow immune tolerance-inducing substances to be released through the accessory channels under the control of physiological sensors input-driven microprocessor as the chute advances into the lumen, the same scheme is applied, but control over chute movement must be continuously variable and precise.

Where movement of the chute is to proceed at a tightly controlled gradual rate tightly coordinated with the delivery of tolerance-inducing substances, the chute-driver must be a servomotor. A chute moved by this means can be interim changed in speed of advancement, can be made to reverse direction and recover a distance already traversed, in short, can change speed and reverse direction at any moment as well as emulate the action of a damped nonsparking plunger solenoid, so that the chute is suddenly ejected to fully obturate the substrate lumen.

This effectively interposes an intervening stage between the bistable settings where the chutes are entirely withdrawn or deployed. When used for permanent diversion, as of urine directly from the ureters in a patient following exenteration or pelvic evisceration, the bistable chute is manually advanced into the ureter at the time of placement and remains there permanently as a prosthesis. When an intervening portion of the lower urinary tract is diseased or missing, diversion can be used to bypass the missing portion. While the lumen of a side-entry diversion jacket or valve is the same or nearly so in internal diameter as the substrate native vessel or ureter the flow through which it is used to divert, the same lumen has applications other than along blood or urine lines. That is, a mainline can be used as would a sideline, or drugline sfor the direct targeting of drugs unrelated to the passage of blood or urine.

### Complication Avoidance in Side-Entry Device Design

The potential complications of encircling a bodily ductus include the provocation of swelling, stenosis, injury to the adventitia, and an atherosclerotic response due to complete enclosure of the adventitia or fibrosa with compression of the fine vasa vasorum and nervorum, the propensity of smaller synthetic lines in the vascular tree to accumulate clot, those in the urinary tract to accumulate crystal, necessitating the frequent replacement of ureteral stents, for example, and the risks associated with an unsecured distal end, to include perforation or incision, considerably abridging freedom of movement, much less participation in heavy labor or sports.

A basic ductus side-entry jacket—that is, one, which unlike a vascular diversion valve—lacks a diversion chute or magnetic layer to draw superparamagnetic micro or nanoparticle carrier bound drugs or radiation shielding sufficient to protect against moderate dose rate radionuclides, for example. To prevent degradation of the substrate ductus, all side-entry jackets are enclosed within spring-hinged hemicylindrical shells heavily perforated as not to completely enclose the substrate ductus. These perforations continue down through the moisture barrier-encapsulated viscoelastic polyurethane foam lining, which temperature responsive, gently conforms to, or invests, the fine nerves and vessels of the adventitia or fibrosa rather than compressing these.

The foam lining accommodates the pulse, changes or irregularities in the external diameter of the substrate vessel, and allows a jacket of given size to be used with vessels of different diameter, and the use of jacket spring hinges, elastic fluid lines, coiled and accordioned as determined by their length, and Bluetooth and transcutaneous energy transferr to avoid hard wiring additionally accommodate growth. So far, pediatric heart transplants tend to remain viable for just under 14 years (see, for example, Dipchand, A. I., Kirk, R., Edwards, L. B., Kucheryavaya, A. Y., Benden, C., and 6 others 2013. “The Registry of the International Society for Heart and Lung Transplantation: Sixteenth Official Pediatric Heart Transplantation Report, 2013, Focus Theme: Age,” Journal of Heart and Lung Transplantation (New York, New York) 32(10): 979-988), when a retransplantation becomes necessary. Given this finite term and the lesser further growth after retransplantation, the incorporation of these features should accommodate the patient over the term of the initial transplant.

To prevent clogging and allow the direct targeting to the jacket and its substrate ductus of medication, such as antimicrobial, antistenotic, antispasmodic, immunosuppressant, opioid, anti-inflammatory, anticoagulant, stem cells, or other therapeutic or line maintenance solutions, for example, every ductus side-entry jacket includes at least one subsidiary or accessory channel, or sideline, connected to a small port at the surface of the body. Tightly targeted direct pipe delivery from a surface port or from the port into a flat pectoral reservoir, then automatically from the reservoir through a line to the jacket at the treatment site without exposing other tissue avoids side effects and interactions-especially beneficial with chemotherapeutics, immunosuppressants, and steroids.

Release from the reservoir is governed by the implant microcontroller or microprocessor, which controls the small reservoir outlet pump or drip stopcock in accordance with a prescription-program, provided data from implanted sensors. The subdermal port includes openings for the mainline and accessory channel or channels. Lines connecting the surface port to a jacket are routed to avoid strangulation of intervening or abrasion of neighboring tissue. Whenever possible, the lines are tunneled subcutaneously. These counter-degradative features and the use of tissue compatible materials mean that ductus side-entry jackets and vascular valves can be used periprocedurally or left in place permanently to continue targeting medication to the treatment site.

Except when an outflow line is needed, as in the diversion of urine to a collection bag, or a direct path to the jacket is needed to insert a miniature cabled device such as an angioscope, laser, intravascular ultrasound probe, or atherectomizer, up to and into the jacket or its ductus, the port is placed subdermally, much as a conventional Mediport® or Portacath,® but smaller and providing multiple openings.

The parts of basic side-entry jackets are shown in FIG. 3 and those of a vascular valve in FIG. 4. Ductus side-entry jackets have a side-connector, or side-stem, with a trepan at the free distal end. To place the jacket about the ductus, the jacket with side-stem or side-connector lock nut loosened is placed about the ductus, and the side-connector attached to a suction pump. When the trepan is abutted against the side of the ductus, the vacuum draws the ductus wall through the trepan, cutting a plug of tissue out of the ductus wall, thus creating an ostium through which the jacket mainline coursing through the side-stem will communicate with the lumen of the ductus. If necessary, a guidewire with hooked free end is used to extract the plug. The foam-lined encircling jacket closes off any path a leak might pursue.

As soon as plug excision is accomplished, leakage of ductus contents through the ostium is minimized if not eliminated by a water jacket which effectively directs a pressurized jet of water against the ostium. With a vascular valve, a thin elastic rubbery valve ostium obturator, or stopper, co-molded with and integral with the chute at its adluminal tip is kept folded as long as the chute, as shown in FIGS. 1A and 2A, remains retracted. When the tip clears the ostium, however, the valve ostium obturator is freed to open, closing off, or obturating, the ostium and thus preventing any, much less clinically significant, leakage of luminal contents.

Placement of an intravascular diversion valve is substantially the same as the placement of a basic jacket with the advantage that, as indicated, the diversion chute has an integral ostium valve ostium obturator at its distal tip. The chute with integral obturator are made of an elastic synthetic medical grade rubbery material such as made by Saint Gobain (Compagnie de Saint-Gobain S. A., La Defense, Courbevoie, France) such that the thin obturator can be folded with the chute retracted into the side-stem to expand under its intrinsic shape memory restorative force when the ostium is cleared, thus occluding the ostium. For sufficient apposition, the edges of diversion chutes and obturators are pliant and feathered. When the chute is fully advanced into the native lumen as shown in the accompanying drawing figures of vascular valves, the compliant obturator with periphery feathered to a thin edge conforms to the curvature of the opposing luminal wall with no intraluminal protrusion as would accumulate clot, or in a ureter, crystal, and the chute seals off the upstream lumen, diverting flow out through the side-stem or side-connector.

Not necessary with a vascular valve, reverse flow of luminal contents out through the ostium and side-stem is prevented by filling the line leading from the port at the body surface to the jacket with water or a more viscid hydrogel, generally containing a ‘cocktail’ of the medication essential to maintain the ductus, the tissue it supplies, or the line itself. Advancement into the lumen of medication is then by injection of an additional bolus. Additionally, with a basic side-entry jacket, following plug removal, the water jacket is available as an accessory channel, or sideline, little susceptible to reverse flow of luminal contents available to bypass the mainline for direct infusion from the surface port into the side-stem and substrate lumen.

Flow through the accessory channel independent of that through the native lumen and mainline, medication flows antegrade, while biopsy test samples are drawn in retrograde flow and out the surface port. Where blocking reverse flow through the side-stem of a basic jacket by filling the mainline with a drug-releasing hydrogel is objectionable, a diversion jacket can be used even though diversion as such is not involved. As an accessory channel, the water jacket can be used bidirectionally to introduce or draw luminal contents, a small preceding bolus of side-stem contents of no therapeutic or diagnostic significance discarded.

Name this

In a one-time vascular valve, full advancement of the chute is signaled to the operator by a clearly audible click when small prominences, or detents, along the underside of the chute base snap into complementary depressions along the raceway. In diversion valves, the accessory channel courses axially beneath the chute to end just short of the downward extension of the valve ostium obturator at the distal end of the chute. As in a basic jacket, additional accessory channels can be added to enter the side-stem.

For a patient with enuresis or nocturia or one who is a public performer beset with frequent urgent onset urination so that diversion is needed on an intermittent basis, the body surface port includes not only a urine outlet seen in the port shown in FIG. 26C through the center of the port to insert a drainage tube to a collection bag but a switch to control a miniature push-pull solenoid with detent at either end of the throw to retain the solenoid plunger in the last push or pull position. The valves on the right and left are not continuously adjustable choke or diversion servovalves but rather solenoid and bistable, that is, entirely extended or retracted, choke solenoid valves with not a curved diversion chute but a planar obstructor or obturator.

Switching the solenoid applies sufficient force causes the plunger to overcome the retaining strength of the detents and move to the opposite position, thereby shifting the diversion chutes from diversion during the night, for example, to non-diversion when facilities are convenient. The solenoids draw current only when actuated, thus conserving battery power. An alternative arrangement, which eliminates the need for electrical operation consists of control knobs on the port to move right and left Bowden cables.

To allow halving the volume of urine collected, hence, the accumulation of weight in the collection bag, a benefit for an athlete, for example, ureteral takeoff can be used unilaterally. To selectively allow halving the volume of urine collected, the diversion chute on one side only is advanced regardless of whether the emplacement is bilateral. In a prosthesis for a wearer with at least one ureter but without a lower urinary tract or portion thereof, hence without urgency to signal the need to void, switching is not incorporated; urine instead allowed to flow into the collection bag at any time.

To allow the wearer to clearly see the port for use the control switches or knobs if present, emptying the collection bag into a bathroom receptacle, and to sterilize the port with soap and a sanitizer, the small port is positioned to one side of the electrolysis-prepared mons pubis. A system with a takeoff from both ureters can outflow through ports at either side of the mons pubis. Generally, the diversion of blood is between different arteries or different veins, not from an artery to a vein or the reverse other as would deliver higher pressure blood into a vein or oxygenated into unoxygenated blood or the reverse.

However, from a surgical standpoint, provided the relative volume of diverted blood results in no significant hypoxia, a more secure course for the connecting mainline may outweigh this generalization. Diversion valves for the precise metered partial diversion of blood flow from the pulmonary artery to one or more pulmonary veins to control pulmonary hypertension in lieu of banding are vascular servovalves, wherein partial advancement and retraction of the chute is by a miniature linear motor or motorized leadscrew. When not part of a more comprehensive prosthetic disorder control system wherein an implanted microprocessor has been prescription-programmed to achieve the optimal homeostatic condition across a set of comorbidities, the degree of adjustment is controlled by an implanted microcontroller on the basis of output from strain gauge and other implanted sensors.

### Implanted Simple and Hierarchical Control Systems

In a more comprehensive system, each morbidity is treated as an arm or channel of an adaptive hierarchical control system (see, for example, Aguilar, J., Cerrada, M., Mousalli, G., Rivas, F., and Hidrobo, F. 2005. ““A Multiagent Model for Intelligent Distributed Control Systems, “pages 191-197; Albus, J., Bostelman, R., Hong, T., Chang, T., Shackleford, W., and Shneier, M. 2006. “The LAGR [Learning Applied to Ground Robots] Project: Integrating Learning into the 4D/RCS [4 Dimensional Remote Control System] Control Hierarchy,” International Conference in Control, Automation and Robotics—ICINCO 06, Setubal, Portugal, available at http://www.nist.gov/customcf/get_pdf.cfm?pub_id=822702; Albus, J. S. 1995. “RCS: A Reference Model Architecture for Intelligent Systems, Association for the Advancement of Artificial Intelligence Technical Report SS-95-02, available at http://aaaipress.org/Papers/Symposia/Spring/1995/SS-95-02/SS95-02- 001.pdf; Albus, J. S. 1993. “A Reference Model Architecture for Intelligent Systems Design,” Chapter 2, pages 27-56 in Antsaklis, P. J. and Passino, K. M., eds., An Introduction to Intelligent and Autonomous Control, Baltimore, Maryland: Wolters Kluwer Academic Publishers, W. 1984. “The Essentials of Hierarchical Control,” in Thoft-Christensen, P. (ed.), System Modelling and Optimization. Lecture Notes in Control and Information Sciences 59:38-61; Findeisen, W.; Bailey, F. N., Brdys, M., Malinowski, K., Tatjewoki, P., and Wozniak, A. 1980. Control and Coordination in Hierarchical Systems, New York, New York: John Wiley and Sons, Issue 9 of the International Series on Applied Systems Analysis, Wiley-Interscience; Meystel, A. M. and Albus, J. S. 2002. Intelligent Systems, New York, New York: John Wiley and Sons) with sensors reporting through subordinate node microcontrollers which coordinate the data each at its respective hierarchical level and pass it up through superior microcontrollers to the microprocessor as master node. The microprocessor controls the outlet pumps or drip stopcocks of small flat drug reservoirs placed subdermally in the pectoral region, replenished through the surface port.

To allow radio remote control, the solenoids or linear motors are adjusted automatically by the microcontroller, of which the power source is usually a battery in the surface port recharged manually or transcutaneously (transdermally) or an implanted capacitive device (see, for example, Bocan, K. N. and Sejdic, E. 2016. Adaptive Transcutaneous Power Transfer to Implantable Devices: A State of the Art Review,” Sensors (Basel) 16(3). pii: E393; Vihvelin, H., Leadbetter, J., Bance, M., Brown, J. A., and Adamson, R, B. 2016. “Compensating for Tissue Changes in an Ultrasonic Power Link for Implanted Medical Devices,” Institute of Electrical and Electronics Engineers Transactions on Biomedical Circuits and Systems 10(2): 404-411; Ben Amar, A., Kouki, A. B., and Cao, H., 2015. “Power Approaches for Implantable Medical Devices,” Sensors (Basel) 15(11): 28889-28914; Ho, J. S., Yeh, A. J., Neofytou, E., Kim, S., Tanabe, Y., Patlolla, B., Beygui, R. E., and Poon, A. S. 2014. “Wireless Power Transfer to Deep-tissue Microimplants,” Online, Proceedings of the National Academy of Sciences of the United States of America 111(22): 7974-7979; Meng, C., Gall, O. Z., and Irazoqui, P. P. 2013. “A Flexible Super-capacitive Solid-state Power Supply for Miniature Implantable Medical Devices,” Biomedical Microdevices 15(6): 973-983; Artan, N., Vanjani, H., Vashist, G., Fu, Z., Bhakthavatsala, S., Ludvig, N., Medveczky, G., and Chao, H. 2010. “A High-performance Transcutaneous Battery Charger for Medical Implants,” Conference Proceedings, Institute of Electrical and Electronic Engineering in Medicine and Biology Society 2010:1581-1584).

When used for sudden compound vascular bypass, or sudden switched-organ, transplantation, multiple diversion valves, some obscured by intervening tissue or close to structures such as the recurrent laryngeal nerve or other readily injured nervous structures, must be switched at the same instant. For this reason, simultaneous switching of the diversion valves, another one-time, or prosthetic application, is by means of tiny damped nonsparking plunger, bistable linear, highly damped nonsparking plunger, or ‘punching’ type solenoids such as those shown in FIGS. 7 and 8 controlled from the same switch by the operator or an assistant. Whether the coiled mainlines are left in or removed, the electrical lines to the solenoids are removed from the recipient before closing.

When the deceased donor continued on life support must be preserved to allow other organs to be used, continuity of perfusion and the prevention of blood loss upon excision of the graft organ is accomplished by anastomosing the vascular stumps. For more extended preservation, the deceased donor can be fitted with an intracorporeal reusable artificial heart, with hormone replacement therapy, warming, and electrolyte surveillance conventional (see, for example, Shetty, V, L., Mali, S. S., Shetty, S. V., and Shinde, P, D. 2017. Op cit.).

With damped nonsparking solenoid-driven valves as applicable in organ transplantation, power consumption is negligible as essential to keep the chutes retracted, not advanced; removing the lines disallows postoperative retraction, and detents lock the bistable valves in the advanced position. In contrast, continuously variable vascular servovalves as used to effect local adjustment in blood pressure require continuous implanted battery recharging by means of transcutaneous, or transdermal, energy transfer (see, for example, Ibrahim, A., Meng. M., and Kiani, M. 2018. “A Comprehensive Comparative Study on Inductive and Ultrasonic Wireless Power Transmission to Biomedical Implants,” Institute of Electrical and Electronics Engineers Sensors Journal 18(9): 3813-3826; Chang, T. C., Weber, M. J., Charthad, J., Baltsavias, S., and Arbabian, A. 2018. “End-to-end Design of Efficient Ultrasonic Power Links for Scaling towards Submillimeter Implantable Receivers,” Institute of Electrical and Electronics Engineers Transactions on Biomedical Circuits and Systems 12(5): 1100-1111; Dinis, H., Colmiais, I., and Mendes, P. M. 2017. “Extending the Limits of Wireless Power Transfer to Miniaturized Implantable Electronic Devices” Micromachines (Basel, Switzerland) 8(12). pii: E359; Agarwal, K., Jegadeesan, R., Guo, Y. X., and Thakor, N. V. 2017. “Wireless Power Transfer Strategies for Implantable Bioelectronics,” Institute of Electrical and Electronics Engineers Reviews in Biomedical Engineering 10:136-161; Yeon, P., Mirbozorgi, S. A., Lim, J., and Ghovanloo, M. 2017. “Feasibility Study on Active Back Telemetry and Power Transmission through an Inductive Link for Millimeter-sized Biomedical Implants,” Institute of Electrical and Electronics Engineers Transactions on Biomedical Circuits and Systems 11(6): 1366-1376; Mirbozorgi. S. A., Yeon, P., and Ghovanloo, M. 2017. “Robust Wireless Power Transmission to mm-sized Free-floating Distributed Implants,” Institute of Electrical and Electronics Engineers Transactions on Biomedical Circuits and Systems 11(3): 692-702; Meng. M. and Kiani. M. 2017. “Design and Optimization of Ultrasonic Wireless Power Transmission Links for Millimeter-sized Biomedical Implants,” Institute of Electrical and Electronics Engineers Transactions on Biomedical Circuits and Systems 11(1): 98-107; Gong, C., Liu, D., Miao, Z., Wang. W., and Li, M. 2017. “An NFC [near-field communication] on Two-Coil WPT [wireless power transfer] Link for Implantable Biomedical Sensors under Ultra-weak Coupling,” Sensors (Basel, Switzerland) 17(6). pii: E1358; Gong, C., Liu, D., Miao, Z., and Li, M. 2017. “A Magnetic-balanced Inductive Link for the Simultaneous Uplink Data and Power Telemetry,” Sensors (Basel, Switzerland) 17(8). pii: E1768; Mao. S., Wang, H., Zhu, C., Mao, Z. H., and Sun, M. 2017. “Simultaneous Wireless Power Transfer and Data Communication Using Synchronous Pulse-controlled Load Modulation,” Measurement (London, England) 109:316-325; Bocan, K. N. and Sejdic, E. 2016. “Adaptive Transcutaneous Power Transfer to Implantable Devices: A State of the Art Review,” Sensors (Basel, Switzerland) 16(3). pii: E393; Ibrahim, A. and Kiani, M. 2016. “A Figure-of-merit for Design and Optimization of Inductive Power Transmission Links for Millimeter-sized Biomedical Implants,” Institute of Electrical and Electronics Engineers Transactions on Biomedical Circuits and Systems10(6): 1100-1111; Meng. M. and Kiani, M. 2016. “Optimal Resonance Configuration for Ultrasonic Wireless Power Transmission to Millimeter-sized Biomedical Implants,” Conference proceedings. Annual International Conference of the IEEE Engineering in Medicine and Biology Society 2016:1934-1937; Ahn, D. and Ghovanloo, M. 2016. “Optimal Design of Wireless Power Transmission Links for Millimeter-sized Biomedical Implants,” Institute of Electrical and Electronics Engineers Transactions on Biomedical Circuits and Systems 10(1): 125-137; Swain, B., Nayak, P. P., Kar, D. P., Bhuyan, S., and Mishra, L. P. 2016. “Wireless Energizing System for an Automated Implantable Sensor,” Review of Scientific Instruments 87(7): 074708).

Only sudden compound vascular bypass, or sudden switched-organ transplantation is applicable to replacement of the native with an artificial heart. Unlike a native heart in the circulatory system of a recipient pending a heart transplant, a sterile artificial heart has no immune significance, so that tolerogenic induction and cross-microchimerization do not, and therewith, metered switch technique does not apply. Implanted in the recipient, blood flow is through the artificial heart and all the tissues of the body, to include the spleen, thymus gland, bone marrow, and lymph nodes. Accordingly, when the artificial heart is exchanged for a real heart, metered transplantation does apply. Prior to reciprocal cross-circulation with a donor, the induction of immune tolerance is by infusion or injection of blood products and drugs.

2b(1)(b). Metered Compound Vascular Bypass, or Metered Switched-Organ Transplantation

Sustaining perfusion and allowing the supplementation or replenishment of essential substances, life support sustained past death mitigates the severity of inflammation and degradation in graft organ quality which death would otherwise cause. However, absent the ability to harvest the graft organ without interruption in normal perfusion, the potential benefit in the ability of life support by any means to sustain graft quality is lost.

Instead, the convention is precisely the reverse of what it should be—life support is shut off and the graft organ excised, causing not only injury due to the loss of perfusion upon death with its inflammatory integrated stress response and ‘cytokine storm,’ but the trauma of excision and reperfusion injury when transplanted, increasing the susceptibility of the graft to failure due to immune intolerance. However, where there is no interruption in normal perfusion, there can be no reperfusion injury. In switch transplantation, the use of life support through the means elaborated above in section 1a, entitled Introduction, General is limited to a deceased donor, not a prospective recipient.

Metered compound vascular bypass, or sudden switched-organ transplantation allows a measure of reciprocal cross-microchimerization between recipient and donor to continue as the graft organ whether a heart, liver, lung, or kidney, for example, is gradually transferred from the circulatory system of the donor into that of the recipient, a factor in the interval over which the transfer is accomplished. The further integration into this process of gene and stem cell therapy to restore a marginal replacement heart as well as assist in the reciprocal cross-chimerization to augment immune tolerance induction is addressed above in section 2a. These measures considerably increase the pool of acceptable hearts for the elderly, who frequently die with a marginal heart that would not be accepted even for placement in another elderly patient.

Currently, an elderly patient will lose a competent heart to an otherwise healthier young patient. Placement of a second heart will allow an infant with an unsurvivably malformed heart that no amount of surgical reconstruction could restore to normal function to survive with closer to, if not normal function, at least as a bridging strategy until a healthier heart can be transplanted orthotopically, preferably, using the metered swtich method, which offers numerous advantages over conventional orthotopic transplantation. Ventricular assist devices available for infants are likely to provide nonpulsatile flow with adverse consequences as would a Fontan reconstruction. The devastating and irreversible sequelae to a heart that malfunctions at the outset of life is reviewed above in section 2a(1), entitled Congenital Defects of the Heart and Other Organs—The Importance of Prompt Intervention.

In both sudden and metered switch orthotopic or single heart transplantation, even though switched between two circulatory systems, both donor and recipient hearts receive and work on a volume of blood associated with an intact circulation system throughout. When placed in the recipient, the donor heart contains a full complement of blood and will support all of the blood in the circulatory system of the recipient, not half that volume as in a double heart transplant, addressed in the section that follows. Accordingly, no intracardiac hypovolemia occurs and no blood transfusion should be necessary. In sudden swtich transplantation, all of the diversion valves are controlled by the operator or an assistant from the same switch and remain open or are closed at the same instant.

By contrast, in metered compound vascular bypass, or sudden switched-organ transplantation, the servovalves are controlled by the implanted microcontroller or microprocessor, and while usually advancing at the same rate rate, are separately controllable. Such discriminatory control allows certain if not all of the servovalves to be retracted if the continued delivery of blood or drugs through these valves has been detected as provoking an adverse reaction so that microcontroller suspends the action while releasing remedial medication to the arterial supply, for example.

Depending upon the resolution, the process is continued until the servovalves are completely open whereupon the innate organ of the recipient is bypassed and all blood flow passed instead to and through the graft organ. The relative proportion of mixture between donor and recipient blood depends upon the amount of time the process continues with the end point for blending being that blood flow through both the recipient and the donor is half innate and half that of the other. Presumably capable of having induced microchimeric alteration in the condition of both donor and recipient, once the procedure has been completed and the donor, continued to be sustained in a life-like condition to preserve the other organs as explained above in section 2a. Background. General, the use of remaining organs or other tissues should be preceded by postprocedural immune testing.

In a metered switch double heart, or heterotopic, transplant, described below in section 2b(1)(b)(b5), entitled Double Heart Metered Compound Vascular Bypass, or Sudden Switched-organ Transplantation [and so on], whereby the recipient retains the native heart to which is then added the heart of the donor (now containing an equal or greater measure of recipient blood obtained during metered immune tolerance induction), the two hearts will share a volume of blood associated with a single circulatory system.

The intracardiac hypovolemia this produces is overcome through a blood transfusion and the execution by the master node microprocessor of a fibrillation-preventive subroutine that includes adjustment of the diversion servovalves connecting the recipient to the donor heart to alternately apportion a full volume of donor/recipient mixed blood to either heart. Should fibrillation eventuate and not be reversed by these measures, the heart or hearts are shocked.

In a single or orthotopic heart transplant, it should be adequate to connect the control microprocessor to the fibrillation sensor components of an implantable cardioverter defibrillator to initiate appropriate valve adjustments and the direct targeting of drugs without the need for the shock generator. The shocking feature of implantable cardioverter defibrillators is, however, potentially injurious and to be avoided. An object in switch transplantation is to preserve the integrity of the graft organ by transplanting it intact, without surgical wounding, which would increase the need for postoperative drugs that might result in complications. When the ventricular absolute stroke volume of the left and/or right ventricle of the native heart is inadequate to drive the output through the connecting intravascular diversion servovalve jacket mainlines to the corresponding vessels of the donor heart, a uni- or biventricular assist device and/or a positive inotrope administered in a dose cognizant of the imminent discarding of the native heart.

In this context, the short term use of an inotrope (see, for example, Machaj, F., Dembowska, E., Rosik, J., Szostak, B., Mazurek-Mochol, M., and Pawlik, A. 2019. “New Therapies for the Treatment of Heart Failure: A Summary of Recent Accomplishments,” Therapeutics and Clinical Risk Management 15:147-155; Mitrovic, V., Forssmann, W. G., Schnitker, J., and Felix, S, B. 2018. “Randomized Double-blind Clinical Studies of Ularitide and Other Vasoactive Substances in Acute Decompensated Heart Failure: A Systematic Review and Meta-analysis,” European Society of Cardiology Heart Failure 5(6): 1023-1034; Psotka, M, A. and Teerlink, J, R. 2017. “Direct Myosin Activation by Omecamtiv Mecarbil for Heart Failure with Reduced Ejection Fraction,” Handbook of Experimental Pharmacology 243:465-490; Tariq, S. and Aronow, W. S. 2015. “Use of Inotropic Agents in Treatment of Systolic Heart Failure,” Online, MDPI [Multidisciplinary Digital Publishing Institute] International Journal of Molecular Sciences (Basel, Switzerland) 16(12): 29060-29068; Amin, A. and Maleki, M. 2012. “Positive Inotropes in Heart Failure: A Review Article,” Heart Asia 4(1): 16-22) can do little damage.

The same pertains to short term use of a left ventricular assist device, which were it used on a long-term basis, would eventually pose complications of problem bleeding, infection, right ventricular failure, capillary injury, device malfunction, and cognitive impairment (see, for example, Rauch, A., Susen, S., and Zieger, B. 2019. “Acquired von Willebrand Syndrome in Patients with Ventricular Assist Device,” Online, Frontiers in Medicine (Lausanne, Switzerland) 6:7; Han, J. and Trumble, D, R. 2019. “Cardiac Assist Devices: Early Concepts, Current Technologies, and Future Innovations,” Bioengineering (Basel, Switzerland) 6(1). pii: E18; Wadowski, P. P., Steinlechner, B., Zimpfer, D., Schloglhofer, T., Schima, H., and 8 others 2019. “Functional Capillary Impairment in Patients with Ventricular Assist Devices,” (Nature Research) Scientific Reports 9(1): 5909; Peng, D. M., Koehl, D. A., Cantor, R. S., McMillan, K. N., Barnes, A. P., and 7 others 2019. “Outcomes of Children with Congenital Heart Disease Implanted with Ventricular Assist Devices: An Analysis of the Pediatric Interagency Registry for Mechanical Circulatory Support (Pedimacs),” Journal of Heart and antistenotic (New York, New York) 38(4): 420-430; Adamski, M. G., Sternak, M., Mohaissen, T., Kaczor, D., Wieronska, J. M., and 8 others 2018. “Vascular Cognitive Impairment Linked to Brain Endothelium Inflammation in Early Stages of Heart Failure in Mice,” Journal of the American Heart Association (Oxford, England) 7(7). pii: e007694; Proudfoot, A. G., Davidson, S. J., and Strueber, M. 2017. “von Willebrand Factor Disruption and Continuous-flow Circulatory Devices,” Journal of Heart and antistenotic (New York, New York) 36(11): 1155-1163; Prinzing, A., Herold, U., Berkefeld, A., Krane, M., Lange, R., and Voss, B. 2016. “Left Ventricular Assist Devices-Current State and Perspectives,” Journal of Thoracic Disease 8(8): E660-E666; Rosenthal, D. N., Almond, C. S., Jaquiss, R. D., Peyton, C. E., Auerbach, S. R., and 9 others 2016. “Adverse Events in Children Implanted with Ventricular Assist Devices in the United States: Data from the Pediatric Interagency Registry for Mechanical Circulatory Support (PediMACS),” Journal of Heart and Lung Transplantation (New York, New York) 35(5): 569-577; Nascimbene, A., Neelamegham, S., Frazier, O. H., Moake, J, L., and Dong, J, F. 2016. “Acquired von Willebrand Syndrome Associated with Left Ventricular Assist Device,” Blood 127(25): 3133-3141; Fendler, T. J., Spertus, J. A., Gosch, K, L., Jones, P. G., Bruce, J. M., and 5 others 2015. “Incidence and Predictors of Cognitive Decline in Patients with Left Ventricular Assist Devices,” Circulation. Cardiovascular Quality and Outcomes 8(3): 285-291; Bhat, G., Yost, G., and Mahoney, E. 2015. “Cognitive Function and Left Ventricular Assist Device Implantation,” Journal of Heart and Lung Transplantation (New York, New York) 34(11): 1398-1405).

Where the long-term use of a biventricular assist device or fully artificial heart is pertinent, an implanted automatic disorder response system can dispel these complications by direct release into the coronaries of an antimicrobial, anti-inflammatory, or anticoagulant, for example, as necessary. Solenoid-driven sudden switch vascular valves allow the graft organ to be perfused by the recipient without interruption in perfusion before it is harvested, eluding the fundamental problem in organ transplantation of ischemia-reperfusion injury.

To this primary source of transplant failure, metered switch technique adds suppression of immune intolerance as the other primary source. Gradually allowing more blood of the recipient to pass through the graft organ and body of the donor, the immune systems of recipient and donor are acclimated to one another or reveal an adverse reaction that must be suppressed before the graft organ is entirely transferred into the circulatory system of the recipient.

In effecting the diversion of blood flow from recipient to the donor and donor organ suddenly, without manual means for immune tolerance induction administered preprocedurally, such valves preclude the ability to bolster recipient immune tolerance by gradually increasing the relative proportion of recipient flow through the donor and graft organ while still in the donor. Metered compound vascular bypass, or sudden switched-organ transplantation differs from sudden compound vascular bypass, or sudden switched-organ transplantation in the extended interval over which blood is diverted to and from the donor.

As more and more native recipient heart outflow is passed to the donor heart to be transplanted, the reciprocal complement of blood is passed from the donor to the recipient heart, so that there is no progressive increase in cardiac hypovolemia, or unloading, in the recipient native heart to be discarded and cardiac hypervolemia, or overloading, in the donor heart as the process continues.

It is essential that both recipient and donor be well ventilated, with potential causes for an arrhythmia such as hypokalemia or hypoxia eliminated. Since in a one-for-one replacement the recipient heart will be discarded, it may be presupposed that the consequences of a slight oxygen undersaturation in the recipient should not cause much concern; however, even a mild hypoxia, whether caused by and heightened in susceptibility to cardiac incompetency, can trigger an arrhythmia (see, for example, Marinheiro, R., Parreira, L., Amador, P., Mesquita, D., Farinha, J., and 5 others 2019. “Ventricular Arrhythmias in Patients with Obstructive Sleep Apnea,” Current Cardiology Reviews 15(1): 64-74; Salama, A., Abdullah, A., Wahab, A., Eigbire, G., Hoefen, R., and 4 others 2018. “Is Obstructive Sleep Apnea Associated with Ventricular Tachycardia? A Retrospective Study from the National Inpatient Sample and a Literature Review on the Pathogenesis of Obstructive Sleep Apnea,” Clinical Cardiology 41(12): 1543-1547; Geovanini, G. and Lorenzi-Filho, G. 2018. “Cardiac Rhythm Disorders in Obstructive Sleep Apnea,” Journal of Thoracic Disease 10 (Supplement 34): S4221-S4230; Fox, H., Bitter, T., Horstkotte, D., and Oldenburg, O. 2017. “Sleep-disordered Breathing and Arrhythmia in Heart Failure Patients,” Sleep Medicine Clinics 12(2): 229-241; Raghuram, A., Clay, R., Kumbam, A., Tereshchenko, L. G., and Khan, A. 2014. “A Systematic Review of the Association Between Obstructive Sleep Apnea and Ventricular Arrhythmias,” Journal of Clinical Sleep Medicine 10(10): 1155-1160; Antzelevitch, C. and Burashnikov, A. 2011. “Overview of Basic Mechanisms of Cardiac Arrhythmia,” Cardiac Electrophysiology Clinics 3(1): 23-45).

If mechanical ventilation has been or is to be provided for the recipient, measures should be taken to dispel any mental dysfunction that often follows, such as a disindustrializing degree of apprehension, depression, or delirium, whether by avoiding the prolonged use thereof, limiting it to intraprocedural, or if this is unavoidable as in a lung or heart-lung transplantation, then providing counteractive medication (see, for example, Turon, M., Fernandez-Gonzalo, S., de Haro, C., Magrans, R., Lopez-Aguilar, J., and Blanch, L. 2018. “Mechanisms Involved in Brain Dysfunction in Mechanically Ventilated Critically Ill Patients: Implications and Therapeutics,” Annals of Translational Medicine 6(2): 30; de Azevedo, J. R., Montenegro, W. S., Rodrigues, D. P., de C Souza, S. C., Araujo, V. F., and 4 others 2017. “Long-term Cognitive Outcomes among Unselected Ventilated and Non-ventilated ICU Patients,” Journal of Intensive Care 5:18; González-López, A., López-Alonso, I., Aguirre, A., Amado-Rodriguez, L., Batalla-Solis, E., and 6 others 2013. “Mechanical Ventilation Triggers Hippocampal Apoptosis by Vagal and Dopaminergic Pathways,” American Journal of Respiratory and Critical Care Medicine 188(6): 693-702).

Another potential cause of tachycardia and hypotension can be hypovolemia secondary to obstruction in the valves or lines (see, for example, Taghavi, S. and Askari, R. 2019. “Hypovolemic Shock,” Treasure Island, Florida: StatPearls Publishing Co., Online at https://www.ncbi.nlm.nih.gov/books/NBK513297/; Sparacino, N., Geninatti, M., and Moore, G. 2011. “Pacemaker Limitation of Tachycardia in Hypovolemic Shock,” Western Journal of Emergency Medicine 12(4): 565-566). A change in the volume of heart inflow or outflow can be prevented by the release into the valve-jackets of a slow heparin or heparinoid drip to avoid the buildup of clot.

Yet another deterrent is to revert to full complement blood flow by fully retracting, or opening, the valves frequently enough to dispel any deviation therefrom as might arise from or during the reciprocal cross-circulation process. While the process continues, a menacing dysrhythmia in both hearts may be prevented or reversed by using pacemaker/cardioverter defibrillators to signal the microcontroller or microprocessor to initiate prevention of recovery measures. In most cases, these costly devices should not be needed once the hearts adapt to their new situation and the overall homeostatic condition of the patient stabilizes.

If so, the additional risk involved in replacing a current technology device or devices not compatible with transdermal or transcutaneous power, or wireless energy transfer to recharge the inmate battery after a half decade or so as necessary (see, for example Han, J I, and Trumble, D, R. 2019. “Cardiac Assist Devices: Early Concepts, Current Technologies, and Future Innovations,” Bioengineering (Basel, Switzerland) 6(1). pii: E18; Bocan, K N. and Sejdic, E. 2016. “Adaptive Transcutaneous Power Transfer to Implantable Devices: A State of the Art Review,” Sensors (Basel, Switzerland) 16(3). pii: E393; Joung, Y, H. 2013. “Development of Implantable Medical Devices: From an Engineering Perspective,” International Neurourology Journal 17(3): 98-106) when the evidence shows that the counteractant provisions of the implant automatic response system itself would have sufficed will be avoided.

Such risk is well documented in the literature (see, for example, Looi, K, L., Gavin, A., Cooper, L., Dawson, L., Slipper, D., and Lever, N. 2019. “Outcomes of Patients with Heart Failure after Primary Prevention ICD Unit Generator Replacement,” Heart Asia 11(1): e011162; Padala, S. K. and Ellenbogen, K, A. 2019. “Primary Prevention ICD Generator at End of Life: to Replace or Not to Replace?,” Heart Asia 11(1): e011167; Wuest, S., Twerenbold, R., Kuhne, M., Reichlin, T., Sticherling, C., Osswald, S., and Schaer, B. 2018. “Reassessment of Cardiovascular Parameters and Comorbidities in Implantable Cardioverter-defibrillator Patients at the Time of First Replacement,” Clinical Cardiology 41(1): 57-62; Barra, S., Goonewardene, M., Heck, P., Begley, D., Virdee, M., and 3 others 2016. “Implantable Cardioverter-defibrillator Elective Generator Replacement: A Procedure for Aall?,” Journal of Interventional Cardiac Electrophysiology 45(2): 209-218; Lewis, K. B., Stacey, D., Carroll, S, L., Boland, L., Sikora, L., and Birnie, D. 2016. “Estimating the Risks and Benefits of Implantable Cardioverter Defibrillator Generator Replacement: A Systematic Review,” Pacing and Clinical Electrophysiology 39(7): 709-722; Madhavan, M., Waks, J. W., Friedman, P. A., Kramer, D. B., Buxton, A. E., and 8 others 2016. “Outcomes after Implantable Cardioverter-defibrillator Generator Replacement for Primary Prevention of Sudden Cardiac Death,” Circulation. Arrhythmia and Electrophysiology 9(3): e003283; Kramer, D. B., Kennedy, K. F., Spertus, J. A., Normand, S, L., Noseworthy, P. A., and 5 others 2014. “Mortality Risk following Replacement Implantable Cardioverter-defibrillator Implantation at End of Battery Life: Results from the NCDR [National Cardiovascular Data Registry],” Heart Rhythm 11(2): 216-221).

When signaled, the microcontroller in a monomorbidly affected patient undergoing a heart transplant or the microprocessor master node controller in a hierarchical control system in a patient with comorbidities, responds to the source of the problem. Any atheromatous lesions of the coronary arteries in both hearts-any in the donor heart not having caused a myocardial infarction and an infarction in the native heart ameliorated to the extent possible before consideration of a transplant-having already been angioplastied, any in the donor heart just before the incision to implant it, and therefore not necessarily eliminated, but reduced in probability as to a source of angioplasty-induced or post-angioplasty arrhythmia (see, for example, Gorenek, B. 2005. “Management of Cardiac Arrhythmias in Post-PCI Patients,” in Gulizia, M. M. (ed.), Emerging Pathologies in Cardiology Conference, Milan, Italy, New York, New York: Springer, pages 231-239; Sigal, S, L., Gellman, J., Anderson, G. M., True, L. D., Chen, Q., and 3 others 1993. “Potentiation of the Vasospastic Response to Angioplasty by Pretreatment with Fluoxetine. A Study in the Atherosclerotic Rabbit,” Arteriosclerosis and Thrombosis 13(6): 907-914; Sigal, S, L., Gellman, J., Sarembock, I. J., La Veau, P. J., Chen, Q. S., Cabin, H. S., and Ezekowitz, M, D. 1991. “Effects of Serotonin-receptor Blockade on Angioplasty-induced Vasospasm in an Atherosclerotic Rabbit Model,” Arteriosclerosis and Thrombosis 11(3): 770-783) can be readily identified and treated once both hearts are in place, identifying a different source of the problem will take the form of using the vascular servovalves to adjust the rate of flow through the heart in- and outflow vessels.

If diagnostic, meaning the source of a well defined arrhythmia is identified, the prescription-program executed by the controller delivers the appropriate corrective measure or measures. Depending upon the source, this will consist of adjusting the diversion servovalves, and/or targeting drugs, and/or electrostimulation to the in- and outflow vessels of either or both hearts. Otherwise, the implanted microcontroller or microprocessor prescription-program initiates an algorithm that executes a combinatorial optimization-based routine to systematically seek out and correct the problem in order of diminishing probability as to cause and source.

This consists of sequentially and simultaneously adjusting the diversion servovalves on the in- and outflow vessels of either and both hearts in various combinations to either side of the setpoints established on the basis of their relative ventricular absolute stroke volumes, which should allow identification of the source. If this furnishes only an indication as to the source but does not afford a complete reversal, then having been reduced to the servovalves on the vessels responsible for the problem, the routine proceeds to systematically adjust the settings of those valves in various combinations.

While under surgical conditions where only the azygos vein is left to return blood to the heart, the azygos factor, or azygos flow principle, shows that a heart continues to function normally with only one tenth the normal venous return for a short time, this pertains to the time needed to perform an intracardiac procedure under hypothermic conditions, and must not be thought of as satisfying the need for sufficient circulation over more than a brief interval (references at section 2b(1)(b)(b5), entitled Heterotopic Double Heart Metered Compound Vascular Bypass, or Switched-organ, Transplantation [and so on]. At even half the normal venous return, normal function should continue over a considerably longer interval.

When applied, the toggles intervene in the independent beating of the hearts to chose alternate systoles from either heart and queue these in one stream as responsive to a higher virtual ‘superheart,’ that is, as responsive to the demand that the two hearts cooperate to discharge a unified performance worthy of one healthy heart. If during nontoggled or free-running of the two hearts a problem arises due to an insufficiency of powerful consecutive ejections in the consolidated outflow train, the implant microcontroller—or in a patient with comorbid disease, the implant master microprocessor overseeing the hierarchical control system-adjusts or toggles the servovalves to alternatively tap off not the setpoint but rather almost the entire in- and outflow of either heart. This need not involve switching after one or a few systoles but must occur frequently enough to preclude intracardiac hypovolemia.

If one heart is stronger than the other, then in addition to the steady state setpoint offset, a bias is applied that favors the systoles of the stronger heart. In this way, alternative systoles of either heart provided with nearly a full volume of blood are queued into the single outflow stream. That either heart has to work half as hard bodes favorably for recovery. The weaker heart does not undergo disuse atrophy and is kept working enough to encourage recouperation. If either heart shows signs of difficulty, the bulk of moving the load is shifted to the unaffected heart. Toggling allows adjustment in the net ejection fraction responsive to the improved health of either or both hearts.

The rate and flow of this movement to either side of the setpoints-essentially the midway point of diversion chute extension and retraction adjusted for a difference in ventricular absolute stroke volumes between the hearts-here referred to as toggling, is initiated. Toggling and its application is addressed below in section 2b(1)(b)(b5), entitled Heterotopic Double Heart Metered Compound Vascular Bypass, or Switched-organ, Transplantation [and so on]. Even though the volume of venous return is limited to that of the one circulatory system plus and additional third or so transferred with the second heart, toggling, switching the greater part of successive systoles to either heart, can allow both hearts to receive and eject a substantially normal complement.

The frequency and duration of toggling is determined by the implanted control microprocessor on the basis of sensor feedback and trial and error until the optimal result is obtained. If different static settings do not end the problem, the microcontroller or microprocessor initiates a test subroutine in the prescription-program whereby the diversion valves are reciprocated, or toggled, to either side of their setpoints at sample rates and displacements until the combination of settings is found that minimizes if not eradicates the ejection deficiency, intracardiac hypovolemia-based arrhythmia, or other dysrhythmia. In less predictable and potentially dangerous cases, a standby cardioverter-defibrillator can shock the disabled heart of hearts back into eurhythmic function.

Post-transplantation therapy for a double heart transplant consists of complication-preventive measures established on the basis of experience gained with conventional heart transplantation, plus novel measures responsive to the physiological peculiarities appurtenant of the end condition. While the implanted automatic disorder response system-which along with the donor controller and extracorporeal master controller participated in the administration of the transplant—can be used to administer conventional support automatically, the administration of novel postoperative support is dependent upon automatic administration by the system.

Conventional support includes the use of immunosuppressants and vasculopathy-preventive medication (statins, mycophenolate mofetil, proliferation signal inhibitors such as everolimus without mycophenolate mofetil, or if combined with a calcineurin inhibitor immunosuppressant, then combined with mycophenolate mofetil, or if tolerated, rapamycin, diltiazem and aspirin optional) (Clerkin, K. J., Ali, Z. A., and Mancini, D. M. 2017. “New Developments for the Detection and Treatment of Cardiac Vasculopathy,” Current Opinion in Cardiology, Online at https://www.ncbi.nlm.nih.gov/pubmed/28212149).

Entrusting the delivery of medication to the implant system allows targeted release through the accessory channels of the servovalves connecting the hearts, and possibly nonjacketing side-entry connectors attached to the visceral pericardium (epicardium), which prevents exposure of the drugs to nontargeted tissue. Nonjacketing side-entry connectors, described and illustrated in U.S. Pat. No. 11,013,858 can also incorporate additional therapeutic capabilities such as electrostimulatory cardioversion by passing current through the anchoring needles, which hollow, can also be used to inject drugs into the myocardium.

Because the hollow anchoring needles can be hollow for the injection of drugs as well as electrified, the administration of shocks can be automatically anesthetized prior to discharge. Effective defibrillation using optogenetics (reference below), which assumes the preoperative optogenetic engineering of myocytes and stabilization of the light source for which nonjacketing side-entry connectors are ideal. Able to stably aim a light source, these connectors should prove indispensable for the practical implementation of optogenetics to avert fibrillation and instate cardioversion in any heart so affected.

As with conventional heart transplantation, should coronary vasculopathy develop, revascularization by means of angioplasty and/or the placement of drug-eluting stents, or coronary artery bypass grafting is unlikely to result in a good outcome (Clerkin, K. J., Ali, Z. A., and Mancini, D. M. 2017, Op cit.; Heublein, B., Haverich, A., and Borst, H, G. 1990. “Long-term Follow-up after Orthotopic Heart Transplantation,” Thoracic and Cardiovascular Surgeon 38(5): 285-290), leaving preferable the orthotopic retransplantation with a good heart, preferably, using the metered switch method.

Targeting immunosuppressants thus minimizes the susceptibility to infection of the rest of the body. In regimen adherence-undependable patients, automatic administration by the implant system also assures correct timing and dosing. However administered, the conventional measures are intended to dispel most if not all allograft infection, coronary vasculopathy, and acute rejection as the most frequent causes for postoperative mortality. In contrast to conventional follow-up, novel follow-up support following a double heart transplant must be executed by the implant system.

Assisted medicinally with antidysrhythmic medication and/or with electrostimulation, and because the novel situation created will induce the hearts separately and together to adapt to the altered physiological conditions, the frequency and extent of diversion chute toggling to increase the load on either heart often enough should subside, reducing power consumption. That combination of frequency and extent of toggling which dispels fibrillation while least retarding adaptation to the condition in the absence of such support is determined empirically through trial and error. Essentially, the automatic system progressively locates the lowest level of toggling that satisfies the need for sufficiency of joined ejection and prevents fibrillation.

Thus, to conserve energy, the prescription-program, using implantable heart beat sensor circuits, or pacemaker/cardioverter defibrillator detection and control timing circuitry, continuously registers whether there is a need for, and if so, executes an algorithm to determine, the most efficient and effective pattern for toggling so that the two hearts work best together. Sought is that combination of antidysrhythmic drugs and toggling that would dispel a dysrhythmia (Tse, G. 2016. “Mechanisms of Cardiac Arrhythmias,” Journal of Arrhythmia 32(2): 75-81).

This favorable expectation is not depended upon when discharging the patient, provided with a belt-worn battery pack containing rechargeable batteries. Until improved batteries become available, once it has been determined safe to do so, this is later replaced by a small body surface port containing lithium-ion coin, or button, cells. If transcutaneously rechargeable implanted batteries are desired, these are placed when the implant disorder response system is, thus avoiding the need for another invasive procedure.

During adaptation, a need for toggling should decrease until toggling has been minimized or found no longer necessary to be continued except when simultaneity of beating by the hearts indicates the need to increase the ejection fraction by reinstituting toggling. The circulatory system as a whole and as an integrated organ system, having been alleviated of an urgent need to adapt to heart failure, is freed to adapt to the optimal combination of operational parameters at which system performance is optimized for the frequency of toggling.

This mutual adaptation of organic and assist mechanisms to find the best steady state condition, with surveillance to detect and adjust for a deviation thereof affords optimal protection from a sudden arrest. The ultimate object sought is that mechanical and medicinal support will eventually become minimal and precautionary. While determined empirically for each patient, more general information concerning the frequency and extent of diversion chute toggling needed to adequately load either heart and the medicinal regimen needed to prevent dysrhythmias and coronary vasculopathy without interfering with innate adaptation warrants laboratory testing.

Postoperatively, the precautionary administration of antidysrhythmic medication applies. Equipped with ductus side-entry jackets and nonjacketing side-entry connectors, the implant system is able to release fluid medication directly into the vascular tree, so that drugs ordinarily designated for intravenous administration such as ibutilide and lidocaine are not excluded. Other antidysrhythmics, such as dofetilide and ranolazine are taken orally unless the patient is regimen adherence-undependable, in which case the drugs are administered in fluid form by the automatic implanted system.

The patient is monitored for a few days to be certain that a dysrhythmia such as torsades does not arise. If the risk of a dysrhythmia is low, oral administration is prescribed. Fecainide carries serious side effects and is best avoided (see, for example, Roka, A., Stead, T. G., Ramlatchan, S. R., Rubero, J. A., and Ganti, L. 2019. “Electrical Cardioversion for Wide Complex Tachycardia,” Online, Cureus (Palo Alto, California) 11(7): e5174; Suffredini, J. M., Rutland, J., Akpunonu, P., Baum, R., Catanzaro, J., and Elayi, C. S. 2019. “Flecainide Toxicity Resulting in Pacemaker Latency and Intermittent Failure to Capture,” American Journal of Case Reports 20:1279-1283). However, this view has been questioned (see, for example, Chauveau, S., Le Vavasseur. O., Morel, E., Dulac. A., and Chevalier, P. 2019. “Flecainide is a Safe and Effective Treatment for Pre-excited Atrial Fibrillation Rapidly Conducted to the Ventricle in Pregnant Women: A Case Series,” European Heart Journal. Case Reports 3(2). pii: ytz066; Aliot, E., Capucci. A., Crijns, H. J., Goette, A., and Tamargo, J. 2011. “Twenty-five Years in the Making: Flecainide is Safe and Effective for the Management of Atrial Fibrillation,” Europace 13(2): 161-173).

Based upon the pharmacokinetics of the drug, the automatic implanted system periodically releases the drug automatically through a ductus side-entry jacket into the portal vein, vena cava, or if topically effective by direct application, then directly into the affected tissue through a nonjacketing side-entry connector. With input from a heart beat sensor circuit, upon detection of a dysrhythmia, the control microprocessor automatically releases medication, such as Vaughan Williams Class I sodium and/or Class III potassium channel blockers to prompt recovery and can deliver cardioverting or defibrillating electrostimulation through the electrified anchoring needles of nonjacketing side-entry connectors directly into the heart.

The patient is not discharged until it has been determined that the probability of a dysrhythmia to too low to justify continued hospitalization, or that were a dysrhythmia to arise, based upon the data received from implanted heart beat sensor circuits, the implanted system would automatically initiate diversion chute toggling first, if unsuccessful in achieving resynchronization, then release antidysrhythmic medication not previously prescribed, and if resynchronization is still not achieved, direct cardioversion stimulation through the electrified anchoring needles of the implanted nonjacketing side-entry connectors and/or a conventional pacemaker/cardioverter-defibrillator.

The process executed subdermally, there exists no risk of skin burns. Shocks can be injurious to the heart, and the system is able to instantly adjust the current level, so that unless the risk of a sudden arrest is imminent, current discharge is not initiated with excessively powerful shocks but rather defibrillation and repolarization of either or both hearts are accomplished using the least electrical power necessary, timed to avoid discharge on the t-waves. Not transthoracic, the current is adjusted accordingly. The direct and automatic application of electrical discharges into the heart averts transthoracic impedance, the need for higher energy shocks resulting in skin burns, and eliminates dependency upon the fortuitous availability of an automated external defibrillator.

Reduction in cardioversion and defibrillation energy consumption both protects the hearts and reduces the energy storage requirement (Quinonez Uribe, R. A., Luther, S., Diaz-Maue, L., and Richter, C. 2018. “Energy-reduced Arrhythmia Termination Using Global Photostimulation in Optogenetic Murine Hearts,” Online, Frontiers in Physiology 9:1651; Burri, H., Moretti, F., and Meyer, P. 2017. “Low-energy Cardioversion of Ventricular Tachycardia: When Less is More,” Indian Pacing and Electrophysiology Journal 17(2): 44-47; Morgan, S. W., Plank, G., Biktasheva, I. V., and Biktashev. V. N. 2009. “Low Energy Defibrillation in Human Cardiac Tissue: A Simulation Study,” Biophysical Journal 96(4): 1364-1373; Ripplinger, C. M., Krinsky, V. I., Nikolski, V. P., and Efimov. I, R. 2006. “Mechanisms of Unpinning and Termination of Ventricular Tachycardia,” American Journal of Physiology. Heart and Circulatory Physiology 291(1): H184-H192; Fotuhi, P. C., Epstein, A. E., and Ideker, R, E. 1999. “Energy Levels for Defibrillation: What is of Real Clinical Importance?,” American Journal of Cardiology 83(5B): 24D-33D; Hsu, W., Lin. Y., Lang, D. J., and Jones, J. L. 1998. “Shock Timing Lowers Transvenous Defibrillation Energy Requirement,” Journal of Electrocardiology 31 Supplement: 35-40).

Once approved for clinical use, optogenetics will offer less injurious resynchronization than that electrostimulatory (see, for example, Back, S, H. and Kowey, P, R. 2019. “Strategies to Reduce Recurrent Shocks Due to Ventricular Arrhythmias in Patients with an Implanted Cardioverter-Defibrillator,” Arrhythmia and Electrophysiology Review 8(2): 99-104; Black, N., D'Souza, A., Wang, Y., Piggins, H., Dobrzynski, H., Morris, G., and Boyett, M, R. 2019. “Circadian Rhythm of Cardiac Electrophysiology, Arrhythmogenesis, and the Underlying Mechanisms,” Heart Rhythm 16(2): 298-307; Bragard, J., Sankarankutty, A. C., and Sachse. F, B. 2019. “Extended Bidomain Modeling of Defibrillation: Quantifying Virtual Electrode Strengths in Fibrotic Myocardium,” Online, Frontiers in Physiology 10:337; Chung, R., Wazni, O., Dresing, T., Chung, M., Saliba, W., Lindsay, B., and Tchou, P. 2019. “Clinical Presentation of Ventricular-Hisian and Ventricular-nodal Accessory Pathways,” Heart Rhythm 16(3): 369-377; Ferenczi, E. A., Tan, X., Huang, C. L. 2019. “Principles of Optogenetic Methods and Their Application to Cardiac Experimental Systems,” Online, Frontiers in Physiology 10:1096; Funken, M., Malan, D., Sasse, P., and Bruegmann, T. 2019. “Optogenetic Hyperpolarization of Cardiomyocytes Terminates Ventricular Arrhythmia,” Online, Frontiers in Physiology 10:498; Goyal. A., Hill, J., and Singhal. M. 2019. “Cardioversion, Pharmacological” Treasure Island, Florida: StatPearls Publishing Company, Online at https://www.ncbi.nlm.nih.gov/books/NBK470536/; Goyal, A., Sciammarella, J. C., Chhabra, L., and Singhal, M. 2019. “Synchronized Electrical Cardioversion,” Treasure Island, Florida: StatPearls Publishing Company, Online at https://www.ncbi.nlm.nih.gov/books/NBK482173/; Mahtani, A. U. and Nair, D, G. 2019. “Supraventricular Tachycardia,” Medical Clinics of North America (Philadelphia, Pennsylvania) 103(5): 863-879; O'Brien, T., Park, M. S., Youn, J. C., and Chung, E. S. 2019. “The Past, Present, and Future of Cardiac Resynchronization Therapy,” Korean Circulation Journal 49(5): 384-399; O'Shea, C., Holmes, A. P., Winter, J., Correia, J., Ou, X., and 6 others 2019. “Cardiac Optogenetics and Optical Mapping-Overcoming Spectral Congestion in All- optical Cardiac Electrophysiology,” Online, Frontiers in Physiology 10:182; Patti, L. and Ashurst, V. 2019. “Rhythm, Tachycardia, Supraventricular (SVT),” Treasure Island, Florida: StatPearls Publishing Co., Online at https://www.ncbi.nlm.nih.gov>books>NBK441972; Sasse, P., Funken, M., Beiert. T., and Bruegmann, T. 2019. “Optogenetic Termination of Cardiac Arrhythmia: Mechanistic Enlightenment and Therapeutic Application?,” Online, Frontiers in Physiology 10:675; Schmidt, A. S., Lauridsen, K. G., Torp. P., Bach, L. F., Rickers, H., and Lofgren, B. 2019. “Maximum-fixed Energy Shocks for Cardioverting Atrial Fibrillation,” European Heart Journal pii: ehz585; Tanaka, H. 2019. “Fluorescence Imaging of the Living Heart for Understanding the Basis of Arrhythmias,” (in Japanese with abstract at Pubmed) Nihon Yakurigaku Zasshi. Folia Pharmacologica Japonica 154(4): 171-177; Thomas, G., Kim, J., and Lerman, B, B. 2019. “Improving Cardiac Resynchronisation Therapy,” Arrhythmia and Electrophysiology Review (London, England) 8(3): 220-227; Upadhyay GA1, Vijayaraman P2, Nayak HM1, Verma N3, Dandamudi G4, and 14 others 2019. “On-treatment Comparison between Corrective His Bundle Pacing and Biventricular Pacing for Cardiac Resynchronization: A Secondary Analysis of the His-SYNC Pilot Trial,” Heart Rhythm 16(12): 1797-1807; Zaglia. T., Di Bona, A., and Mongillo, M. 2019. “A Light Wand to Untangle the Myocardial Cell Network,” Methods and Protocols 2(2). pii: E34; Arnold, A. D., Shun-Shin, M. J., Keene. D., Howard, J. P., Sohaib, S. M. A., and 15 others 2018. “His Resynchronization versus Biventricular Pacing in Patients with Heart Failure and Left Bundle Branch Block,” Journal of the American College of Cardiology 72(24): 3112-3122; Boyle, P. M., Karathanos, T. V., and Trayanova, N, A. 2018. “Cardiac Optogenetics: 2018,” Journal of American College of Cardiology. Clinical Electrophysiology 4(2): 155-167; Boyle, P. M., Murphy, M. J., Karathanos, T. V., Zahid, S., Blake, R, C. 3rd, and Trayanova, N, A. 2018. “Termination of Re-entrant Atrial Tachycardia via Optogenetic Stimulation with Optimized Spatial Targeting: Insights from Computational Models,” Journal of Physiology 596(2): 181-196; Brubaker. S., Long. B., and Koyfman, A. 2018. “Alternative Treatment Options for Atrioventricular-nodal-reentry Tachycardia: An Emergency Medicine Review,” Journal of Emergency Medicine 54(2): 198-206; Cao, Z. Z., Tian, Y. J., Hao. J., Zhang, P. H., Liu, Z. P., and 4 others 2018. “Barbaloin Inhibits Ventricular Arrhythmias in Rabbits by Modulating Voltage-gated Ion Channels,” Acta Pharmacologica Sinica 39(3): 357-370; Chatterjee, N, A. and Heist, E. K. 2018. “Cardiac Resynchronization Therapy-Emerging Therapeutic Approaches,” Current Treatment Options in Cardiovascular Medicine 20(3): 20; Gruber, A., Edri. O., and Gepstein, L. 2018. “Cardiac Optogenetics: The Next Frontier,” Europace 20(12): 1910-1918; Kaufmann, M. R., Mckillop, M. S., Burkart, T. A., Panna, M., Conti, J. B., and Miles, W. M. 2018. “Iatrogenic Ventricular Fibrillation after Direct-current Cardioversion of Preexcited Atrial Fibrillation Caused by Inadvertent T-wave Synchronization,” Texas Heart Institute Journal 45(1): 39-41; Majumder. R., Feola, I., Teplenin, A. S., de Vries, A. A., Panfilov, A. V., and Pijnappels. D, A. 2018. “Optogenetics Enables Real-time Spatiotemporal Control over Spiral Wave Dynamics in an Excitable Cardiac System,” Elife 7. pii: e41076; Schneider-Warme, F. 2018. “The Power of Optogenetics: Potential in Cardiac Experimental and Clinical Electrophysiology,” Herzschrittmachertherapie und Elektrophysiologie (Heidelberg, Germany) 29(1): 24-29; Semenov, I., Grigoryev, S., Neuber, J. U., Zemlin, C. W., Pakhomova, O. N., Casciola, M., and Pakhomov. A, G. 2018. “Excitation and Injury of Adult Ventricular Cardiomyocytes by Nano- to Millisecond Electric Shocks,” (Nature Research) Scientific Reports 8(1): 8233; Anantharaman, V., Wan, P. W., Tay. S. Y., Manning, P. G., Lim, S. H., and 5 others 2017. “Role of Peak Current in Conversion of Patients with Ventricular Fibrillation,” Singapore Medical Journal 58(7): 432-437; Connolly, A. J., Vigmond, E., and Bishop, M. J. 2017. “Bidomain Predictions of Virtual Electrode-induced Make and Break Excitations around Blood Vessels,” Online, Frontiers in Bioengineering and Biotechnology 5:18: Connolly, A., Vigmond, E., and Bishop, M. 2017. “Virtual Electrodes around Anatomical Structures and Their Roles in Defibrillation,” Online, PLoS [Public Library of Science] One 12(3): e0173324; Ellenbogen, K, A. Wilkoff, B, L., Neal, K. G., Lau, C.-P. and Angelo, A. (eds.) 2017. Clinical Cardiac Pacing, Defibrillation, and Resynchronization Therapy, Philadelphia, Pennsylvania: Elsevier; Han, F. T., Riles, E. M., Badhwar, N., and Scheinman, M. M. 2017. “Clinical Features and Sites of Ablation for Patients with Incessant Supraventricular Tachycardia from Concealed Nodofascicular and Nodoventricular Tachycardias,” Journal of the American College of Cardiology. Clinical Electrophysiology 3(13): 1547-1556; Jones, S., Lumens. J., Sohaib, S. M. A., Finegold, J. A., Kanagaratnam, P., and 9 others with 32 collaborators 2017. “Cardiac Resynchronization Therapy: Mechanisms of Action and Scope for Further Improvement in Cardiac Function,” Europace 19(7): 1178-1186; Lundqvist, C. B., Potpara. T. S., and Malmborg, H. 2017. “Supraventricular Arrhythmias in Patients with Adult Congenital Heart Disease,” Arrhythmia and Electrophysiology Review 6(2): 42-49; Monteiro, L. M., Vasques-Novoa, F., Ferreira, L., Pinto-do-O. P., and Nascimento, D. S. 2017. “Restoring Heart Function and Electrical Integrity: Closing the Circuit,” Nature Partner Journals Regenerative Medicine 2:9; Nyns, E, C. A., Kip. A., Bart, C. I., Plomp. J. J., Zeppenfeld, K., and 3 others 2017. “Optogenetic Termination of Ventricular Arrhythmias in the Whole Heart: Towards Biological Cardiac Rhythm Management,” European Heart Journal 38(27): 2132-2136; Pianca, N., Zaglia. T., and Mongillo, M. 2017. “Will Cardiac Optogenetics Find the Way through the Obscure Angles of Heart Physiology?,” Biochemical and Biophysical Research Communications 482(4): 515-523; Schmidt, A. S., Lauridsen, K. G., Adelborg. K., Torp. P., Bach, L. F., and 5 others 2017. “Cardioversion Efficacy Using Pulsed Biphasic or Biphasic Truncated Exponential Waveforms: A Randomized Clinical Trial,” Journal of the American Heart Association (Oxford, England) 6(3). pii: e004853; Tanaka, H., Matsuyama, T. A., and Takamatsu, T. 2017. “Towards an Integrated Understanding of Cardiac Arrhythmogenesis—Growing Roles of Experimental Pathology,” Pathology International 67(1): 8-16; Varghese, F., Neuber, J. U., Xie, F., Philpott, J. M., Pakhomov, A. G., and Zemlin, C. W. 2017. “Low-energy Defibrillation with Nanosecond Electric Shocks,” Cardiovascular Research 113(14): 1789-1797; Abboud, J. and Ehrlich, J, R. 2016. “Antiarrhythmic Drug Therapy to Avoid Implantable Cardioverter Defibrillator Shocks,” Arrhythmia and Electrophysiology Review 5(2): 117-121; Bibas, L., Levi, M., and Essebag, V. 2016. “Diagnosis and Management of Supraventricular Tachycardias,” Canadian Medical Association Journal 188(17-18): E466-E473; Bruegmann, T., Boyle, P. M., Vogt, C. C., Entcheva, E. and Bub, G. 2016. “All-optical Control of Cardiac Excitation: Combined High-resolution Optogenetic Actuation and Optical Mapping,” Journal of Physiology 594(9): 2503-2510; Guevara, M. R., Shrier, A., Orlowski, J., and Glass. L. 2016. “George Ralph Mines (1886-1914): The Dawn of Cardiac NOnlinear Dynamics,” Journal of Physiology 594(9): 2361-2371; Karathanos, T. V., Arevalo, H. J., and 3 others 2016. “Optogenetic Defibrillation Terminates Ventricular Arrhythmia in Mouse Hearts and Human Simulations,” Journal of Clinical Investigation 126(10): 3894-3904; Karathanos, T. V., Bayer, J. D., Wang, D., Boyle, P. M., and Trayanova, N, A. 2016. “Opsin Spectral Sensitivity Determines the Effectiveness of Optogenetic Termination of Ventricular Fibrillation in the Human Heart: A Simulation Study,” Journal of Physiology 594(23): 6879-6891; Padeletti, L., Pieragnoli, P., Ricciardi, G., Innocenti, L., Checchi, L., and 4 others 2016. “Simultaneous His Bundle and Left Ventricular Pacing for Optimal Cardiac Resynchronization Therapy Delivery: Acute Hemodynamic Assessment by Pressure-Volume Loops,” Circulation. Arrhythmia and Electrophysiology 9(5). pii: e003793; Richter, C., Christoph, J., Lehnart, S. E., and Luther, S. 2016. “Optogenetic Light Crafting Tools for the Control of Cardiac Arrhythmias,” Methods in Molecular Biology 1408:293-302; Tse. G., Lai, E. T., Lee, A. P., Yan, B. P., and Wong. S, H. 2016. “Electrophysiological Mechanisms of Gastrointestinal Arrhythmogenesis: Lessons from the Heart” Online, Frontiers in Physiology 7:230; Brown, T. R., Krogh-Madsen, T., and Christini, D. J. 2015. “Computational Approaches to Understanding the Role of Fibroblast-Myocyte Interactions in Cardiac Arrhythmogenesis,” Online, BioMed Research International 2015:465714; Lustgarten, D, L., Crespo, E. M., Arkhipova-Jenkins, I., Lobel. R., Winget. J., and 3 others 2015. “His-bundle Pacing versus Biventricular Pacing in Cardiac Resynchronization Therapy Patients: A Crossover Design Comparison,” Heart Rhythm 12(7): 1548-1557; Zaglia. T., Pianca, N., Borile, G., Da Broi. F., Richter, C., and 6 others 2015. “Optogenetic Determination of the Myocardial Requirements for Extrasystoles by Cell Type-specific Targeting of ChannelRhodopsin-2,” Online, Proceedings of the National Academy of Sciences of the United States of America 112(32): E4495-E4504; Ambrosi, C. M., Klimas. A., Yu, J., and Entcheva, E. 2014. “Cardiac Applications of Optogenetics,” Progress in Biophysics and Molecular Biology 115(2-3): 294-304; Bingen, B. O., Engels, M. C., Schalij. M. J., Jangsangthong, W., Neshati, Z., and 6 others 2014. “Light-induced Termination of Spiral Wave Arrhythmias by Optogenetic Engineering of Atrial Cardiomyocytes,” Cardiovascular Research 104(1): 194-205; Jaiswal, A. and Goldbarg, S. 2014. “Dofetilide Induced Torsade de Pointes: Mechanism, Risk Factors, and Management Strategies,” Indian Heart Journal 66(6): 640-648; Nijjer, S. S., Sohaib. S. M., Whinnett, Z. I., and Lefroy, D, C. 2014. “Management of Supraventricular Tachycardias,” British Journal of Hospital Medicine (London, England) 75(2): C26-C28; Sohinki, D. and Obel, O, A. 2014. “Current Trends in Supraventricular Tachycardia Management,” Ochsner Journal 14(4): 586-595; Trayanova, N. A. and Rantner, L. J. 2014. “New Insights into Defibrillation of the Heart from Realistic Simulation Studies,” Europace 16(5): 705-713; Abilez, O. J. 2013. “Optogenetic LED Array for Perturbing Cardiac Electrophysiology,” Proceedings of the Annual International Conference of the Institute of Electrical and Electronics Engineers Engineering in Medicine and Biology Society 2013:1619-1622; Entcheva, E. 2013. “Cardiac Optogenetics,” American Journal of Physiology. Heart and Circulatory Physiology 304(9): H1179-H1 191; Williams, E. S. and Viswanathan, M. N. 2013. “Current and Emerging Antiarrhythmic Drug Therapy for Ventricular Tachycardia,” Cardiology and Therapy 2(1): 27-46; Baquero, G. A., Banchs, J. E., Depalma, S., Young, S. K., Penny-Peterson, E. D., and 4 others 2012. “Dofetilide Reduces the Frequency of Ventricular Arrhythmias and Implantable Cardioverter Defibrillator Therapies,” Journal of Cardiovascular Electrophysiology 23(3): 296-301; Darragh, K. M., Manoharan, G., Navarro, C., Walsh, S., Allen, J. D., Anderson, J. M., and Adgey, A. J. 2012. “Synchronized Defibrillation for Ventricular Fibrillation,” European Heart Journal. Acute Cardiovascular Care 1(4): 285-290; Niles, D. E., Nishisaki. A., Sutton, R. M., Brunner, S., Stavland. M., and 5 others 2010. “Analysis of Transthoracic impedance during Real Cardiac Arrest Defibrillation Attempts in Older Children and Adolescents: Are Stacked-shocks Appropriate?,” Resuscitation 81(11): 1540-1543; Sucu, M., Davutoglu, V., and Ozer, O. 2009. “Electrical Cardioversion,” Annals of Saudi Medicine 29(3): 201-206; Walker, R. G., Koster, R. W., Sun, C., Moffat. G., Barger, J., Dodson, P. P., and Chapman, F. W. 2009. “Defibrillation Probability and Impedance Change between Shocks during Resuscitation from Out-of-hospital Cardiac Arrest,” Resuscitation 80(7): 773-777; Fotuhi, P. C., Epstein, A. E., and Ideker, R, E. 1999. “Energy Levels for Defibrillation: What is of Real Clinical Importance?,” American Journal of Cardiology 83(5B): 24D-33D; Zivin, A., Souza, J., Pelosi, F., Flemming, M., Knight, B. P., and 3 others 1999. “Relationship between Shock Energy and Postdefibrillation Ventricular Arrhythmias in patients with Implantable Defibrillators,” Journal of Cardiovascular Electrophysiology 10(3): 370-377; Heavens, J. P., Cleland, M. J., Maloney, J. P., and Rowe, B, H. 1998. “Effects of Transthoracic Impedance and Peak Current Flow on Defibrillation Success in a Prehospital Setting,” Annals of Emergency Medicine 32(2): 191-199; Hsu. W., Lin, Y., Heil, J. E., Jones, J., and Lang, D. J. 1997. “Effect of Shock Timing on Defibrillation Success,” Pacing and Clinical Electrophysiology 20(1 Part 2): 153-157; Dillon, S. M. 1992. “Synchronized Repolarization after Defibrillation Shocks. A Possible Component of the Defibrillation Process Demonstrated by Optical Recordings in Rabbit Heart,” Circulation 85(5): 1865-1878; Kerber, R. E., Kienzle, M. G., Olshansky, B., Waldo, A, L., Wilber, D., and 5 others 1992. “Ventricular Tachycardia Rate and Morphology Determine Energy and Current Requirements for Transthoracic Cardioversion,” Circulation 85(1): 158-163).

If post-transplantation dysrhythmia is persistent, intractable, the less competent of the two hearts is removed and that remaining examined to determine whether it had recovered sufficiently to serve as the sole heart. If not, the patient is entered onto the waiting list for a healthy replacement heart. The implant system already in place, the new heart is transplanted using the metered switch method by reciprocal cross-circulation with the stronger of the two hearts.

In a metered switch orthotopic heart transplant with native and replacement hearts similar in size where the donor heart would almost always exhibit a higher absolute stroke volume, the point of equal blood volume exchange in reciprocal cross-circulation before the servovalve diversion chutes are almost fully extended to considerably reduce the volume provided to the native and considerably increase the volume provided to the replacement heart is offset from the half-way point according to the degree of disparity in the ventricular absolute stroke volumes.

At this point, the blood entering and exiting each heart is half that of the donor and half that of the recipient. At the start of blood diversion from the recipient to the donor, the relative proportion of blood volume through the graft organ contributed by the recipient is least and that contributed by the donor greatest, and this relative proportion is gradually changed, but both hearts receive the normal complement of blood. Half the normal volumetric flow at the midway point in a one-for-one orthotopic heart transplant is transient and fundamentally distinct from the half-way point when a second heart is transplanted into the recipient. In the latter case, blood delivery to both the native or orthotopic and heterotopic grafts would be half as well, but absent measures to be described, this half blood volume would not be transient but fixed. This extent is adjusted for a significant degree of disparity in the ventricular absolute stroke volumes of the hearts.

Cardioverter/defibrillators are placed, at least in some cases, recoverable following an initial period during which the hearts adapt to the changed situation. The same applies where a good replacement heart is not available when two extended criteria donors, or less than ideal hearts are transplanted intact to replace a native heart more severely impaired than the two in cooperation In this case, it will usually be necessary to position one of the hearts heterotopically in the abdomen or subrenally; however, unlike heterotopic positioning of a heart in animal testing of the immune system, supply and drainage through the heterotopically positioned heart is by vessel-to-vessel connection with the orthotopically positioned heart so that the pattern of flow through the heterotopic heart is normal.

When the prospective recipient is not in extremis and time allows, the pair of replacement hearts can be transplanted in successive operations, the second delayed to allow recovery. When the need for a new heart is urgent and two hearts able to achieve competent ejection only when paired or working in tandem are available, the native heart of a donor on somatic support can be paired by switch-transplanting a second heart into the donor to work with the native heart. The pre-paired donor hearts are then switch transplanted into the recipient in a single operation.

As addressed in section 2b(1)(b)(b1), entitled Endothelial Drumming, to follow, modulation of volume delivery in coordination with the pulse can be put to additional uses. As an additional precaution, or ‘bailout,’ should it become necessary, the sustainment of regularity is additionally supported by the temporary placement of wearable cardioverter/defibrillators on both recipient and donor. The object in a gradual transition from the circulatory system of the donor into that of the recipient is to promote immune tolerance induction as the organ is transferred between the two.

The duration of the process is contingent upon the amenability of the organ transplanted as immune tolerance induction prone, or pro-tolerogenic (protolerogenic), such as the liver and kidneys, or tolerance resistant, such as the heart and lungs (see, for example, Hull. T. D., Benichou, G., and Madsen, J, C. 2019. Op cit. above in section 2a; Madariaga, M, L., Kreisel, D., and Madsen, J. C. 2015. “Organ-specific Differences in Achieving Tolerance,” Current Opinion in Organ Transplantation 20(4): 392-399).

In addition to the combined transplantation of organs simultaneously or sequentially because of an inherent interdependency such that the well-being of the one is contingent upon that of the other so that both have become impaired, or unless both are replaced, the graft organ will become sick as well, protolerogenic organs may be combined with tolerance resistant organs in the expectation that induction elsewhere in the body will bolster tolerance in that resistant. An advantage in metered over sudden compound vascular bypass, or sudden switched-organ transplantation is that the exposure of donor and recipient to the allogeneic tissue of one another increases in relative proportion as the blood of each increases in the mix.

Except in an emergency that necessitates sudden compound vascular bypass, or sudden switched-organ transplantation, the different organs are transplanted using the metered switch technique. A more intense immunological confrontation in sudden compound vascular bypass, or sudden switched-organ transplantation is preprocedurally avoided through the administration of immune tolerance induction medication and can be additionally ameliorated by intentionally introducing a further chimerizing factor, such as a donor-recipient exchange of glandular tissue or an additional organ amenable to tolerance induction, such as a third heterotopically positioned kidney from the same donor.

At least when the different organs are taken from the same donor, the adjuvant effect may be due to the increase in common antigen load. “Rejection rates for allografts co-transplanted with donor-specific primary liver, kidney, and heart allografts are significantly lower than rejection rates for allografts transplanted alone,” (Rana, A., Robles, S., Russo, M. J., Halazun, K. J., Woodland, D. C., and 3 others 2008. “The Combined Organ Effect: Protection against Rejection?,” Annals of Surgery (Philadelphia, Pennsylvania) 248(5): 871-879). In a simple replacement, or one- for-one, heart transplant, the procedure ends with all inflow into and outflow out of the replacement heart which is neither over- or underloaded.

The diversion chutes fully retracted, the jackets are left in place whether the lines and jackets are allowed to remain as prostheses or the stumps are anastomosed. By contrast, in a one-for-two procedure, where a good replacement heart cannot be obtained, if not transplanted the patient will die, and the cost of an assist device and its subsequent upkeep is much greater than that for a viable transplant, the diseased heart is either supplemented by the addition of a second imperfect but stronger heart or replaced with two less impaired hearts which together are able to achieve adequate ejection and pumping power. With such an arrangement, normovolemic cardiac flow through both hearts is obtained by fully retracting all four diversion chutes and anastomosing the stumps.

Whereas in a two-for-one procedure this is done in an adult to achieve intracardiac normovolemia with the expectation that a follow-up transplantation will not prove necessary, in a neonate or infant, replacement of an unsurvivably malformed heart with two somewhat impaired, size-mismatched, or ventricular absolute stroke volume discrepant hearts is done as a bridging strategy pending the availability of a good heart. However, as the longevity of a good replacement heart is limited, this bridge is retained unless and until a need for retransplantation arises. Two imperfect transplant hearts which together approximate the function of one oversized transplant heart to generate pulmonary hypertension and vascular resistance should spontaneously adapt to the lesser load over a short term during which serious complications such as convulsions and coma can be controlled.

Especially in the very young, postoperative adaptation of an oversized heart or two hearts with combined ventricular absolute stroke volume higher should attenuate less significant pulmonary vascular hypertension (‘big heart or hyperperfusion syndrome”), especially since growth will also call for increased pressure in that the patient will ‘grow into’ the heart at the same time that the heart will lessen the volume and pressure of its output (see, for example, John, M. and Bailey, L. L. 2018, Op cit. in section 2a, entitled Background of the Invention. General; Shin, H. J., Jhang, W. K., Park, J. J., Yun, T. J., Kim, Y. H., and 3 others 2011. “Heart Transplantation in Pediatric Patients: Twelve-year Experience of the Asan Medical Center,” Journal of Korean Medical Science 26(5): 593-598; Razzouk, A. J., Johnston, J. K., Larsen, R, L., Chinnock, R. E., Fitts. J. A., and Bailey L. L. 2005. “Effect of Oversizing Cardiac Allografts on Survival in Pediatric Patients with Congenital Heart Disease,” Journal of Heart and Lung Transplantation (New York, New York) 24(2): 195-199; Kertesz, N. J., Gajarski, R. J., Towbin, J. A., and Geva, T. 1995. “Effect of Donor-recipient Size Mismatch on Left Ventricular Remodeling after Pediatric Orthotopic Heart Transplantation,” American Journal of Cardiology 76(16): 1167-1172, also cited above in section 2. Background of the Invention. a. General, and below in section (b2), entitled Antepartum Compound vascular bypass, or sudden switched-organ Transplantation of the Heart; Reichart, B. 1992. “Size Matching in Heart Transplantation,” Journal of Heart and Lung Transplantation (New York, New York) 11(4 Part 2): S199-S202; see also sections 2b(1)(b)(b2) below entitled Prenatal Compound Vascular Bypass, or Switched-organ Transplantation of the Heart, and 2b(1)(b)(b3). Neonatal and Infant Compound Vascular Bypass, or Switched-organ Transplantation of the Heart).

In conventional orthotopic heart transplantation, an oversized heart poses a less challenging problem of being fit into the central compartment of the mediastinum and more challenging problem of generating main pulmonary artery backup pressure, or excessive volume delivery-based pulmonary hypertension. The early adverse sequelae of coma or convulsions is, however, medically controllable until the heart adapts to its new situation by relaxing and reducing its ventricular absolute stroke volume. As with an oversized heart, a double heart transplant for which mediastinal space is lacking can still be orthotopically positioned provided the sternum is not closed. If insufficient space in the chest is found as an unexpected contingency, sternal closure can be postponed to allow time for a partial right pneumonectomy (right pneumectomy, right pulmonectomy) to provide sufficient space.

Unlike a conventional heterotopic heart transplant where smaller size with reduced ventricular absolute stroke volume bodes a poor outcome, in double heart transplantation, where the best location to place the second intact heart, small size is serviceable by the direct pipe-targeting capability of the implanted disorder response system and minimizes the extent of right pneumonectomy needed to place the second heart. Tangentially, in the hospital, were it necessary, sternal closure can be deferred for a period of months (Shin, H. J., Jhang, W. K., Park, J. J., Yun, T. J., Kim, Y. H., and 3 others 2011. Op cit. preceding paragraph).

Provided the recipient can tolerate the partial pneumonectomy, placement in the chest should always be chosen. The donor heart will never be larger than this resolution will allow. If placement in the chest is not feasible, then because native and donor hearts remain intact, not unified as in a conventional heterotopic transplant, the double heart transplant offers the option of separately positioning the donor heart infrarenally with a heart cage described in section 2b(1)(b)(b6), entitled Heart Cage for the Infrarenal Abdominal Positioning of a Second Heart, or within the abdomen, pelvis, or least preferred, in the upper abdomen. The implications of a heart cage for provoking a foreign body reaction or the formation of adhesions are addressed above in section 2a to include the subsection entitled Prevention of Adhesions.

As indicated, the addition of a heart with a higher ventricular absolute stroke volume, to be expected in an oversized heart, tends to result in blood pressure-related complications over a brief interval, after which the heart adapts to its new milieu with less exertion. For all practical purposes, an oversized heart will have what is at least initially, too high a ventricular absolute stroke volume, while an undersized heart will have what is at least initially, too low a ventricular absolute stroke volume.

An undersized heart similarly has the capacity to adapt to increased demand placed on it, so that double heart transplantation expands the pool of both over- and undersized hearts beyond that intrinsic in the ability of the heart to adapt, in that the concurrent placement of a normal along with a smaller heart makes it possible for the latter to be even smaller, two small hearts can approximate the output of a normally sized heart, and a smaller heart should complement an oversized heart (see, for example, Delmo Walter, E. M., Huebler, M., Schubert, S., Lehmkuhl, H., Weng. Y., Berger, F., and Hetzer, R. 2012. “Influence of Size Disparity of Transplanted Hearts on Cardiac Growth in Infants and Children,” Journal of Thoracic and Cardiovascular Surgery 143(1): 168-177; Delmo Walter, E. M., Huebler, M., Stamm. C., Alexi-Meskishvili, V., Weng. Y., Berger, F., and Hetzer, R. 2011. “Adaptive Growth and Remodeling of Transplanted Hearts in Children,” European Journal of Cardiothoracic Surgery 40(6): 1374-1383; Mather, P. J., Jeevanandam. V., Eisen, H., Pina, I, L., Margulies, K. B., and 3 others 1995. “Functional and Morphologie Adaptation of Undersized Donor Hearts after Heart Transplantation,” Journal of the American College of Cardiology 26(3): 737-742; Jeevanandam, V., Mather, P., Furukawa, S., Todd, B., Regillo. T., and 3 others 1994. “Adult Orthotopic Heart Transplantation Using Undersized Pediatric Donor Hearts. Technique and Postoperative Management,” Circulation 90(5 Part 2): 1174-1177).

While usable apart from an implanted automatic response system, the inclusion thereof allows constant monitoring for signs of rejection or secondary complications and can be programmed to automatially pipeline target remedial agents directly to the site or sites requiring treatment. Substituting sudden switch valves for continuously variable vascular servovalves in the same positions confers this capability. As the relative proportion of donor blood is increased, the automated system used to control the process detects and responds to any adverse alloreactions. The metered compound vascular bypass, or sudden switched-organ technique differs from the sudden compound vascular bypass, or sudden switched-organ technique in using continuously variable vascular servovalves rather than solenoids actuated jointly from a single plunger switch. This allows the transfer of the graft organ to be carried out in a controlled gradual period during which the blending of the blood of donor and recipient is used to achieve a measure of immune tolerance.

To allow the gradual release of supplementary immunosuppressant immune tolerance-inducing agents such as anti-thymocyte globulin during the intervening stage, during which the bloodstreams of the recipient and donor as shown at the center of FIGS. 1B and 2B are blended, metered compound vascular bypass, or sudden switched-organ transplantation deploys the diversion chutes over an intervening interval, effectively interposing a stage between the starting and end points in sudden compound vascular bypass, or sudden switched-organ transplantation. During this intervening or central stage, the valves pass the circulation of both donor and recipient, which is shared between the graft organ and the rest of the circulatory systems of both donor and recipient, to include the supply and drainage to the spleens and thymus glands.

Metered compound vascular bypass, or sudden switched-organ transplantation thus interposes an intervening stage between the starting and end points of sudden compound vascular bypass, or sudden switched-organ transplantation. Metered compound vascular bypass, or sudden switched-organ transplantation applies to solid organ transplantation in prenates, neonates, infants, and adults. The duration of metered switching and the donor-recipient immune tolerance induction it can achieve depends upon that sustainable with the deceased donor. Intensive vigilance by hematologists and internists will extend the interval over which a deceased donor can provide suitable organs. The interposed interval is used to gradually transfer circulation from the native organ of the recipient to and through the donor graft organ in a continuous manner so that the relative proportion of blood flowing through the graft organ is increasingly that of the recipient.

To intentionally increase the shear stress against the endothelia of both donor and recipient to achieve increased assimilation of one another's cells and donor-specific transfusion-induced suppression of allograft rejection if not reciprocal cross transfusion microchimerization of the graft organ and by the donor of recipient cells, the blood pressures of recipient and donor can be artificially elevated somewhat during transfer of the graft (Ueta, H., Kitazawa, Y., Sawanobori, Y., Ueno, T., Ueha. S., Matsushima, K., and Matsuno, K. 2018, Op cit. above in section 2a: Asari, S., Itakura, S., Rawson, J., Ito. T., Todorov. I., and 5 others 2011, Op cit.; Racine, J., Wang, M., Zhang, C., Lin, C, L., Liu, H., and 3 others 2011. “Induction of Mixed Chimerism with MHC-mismatched but not Matched Bone Marrow Transplants Results in Thymic Deletion of Host-type Autoreactive T-cells in NOD [nonobese diabetic] mice,” Diabetes 60(2): 555-564).

Means to measure shear stress in the vascular system already exist (see, for example, Zhang, B., Ma. Y., and Ding, F. 2018. “Evaluation of Spatial Distribution and Characterization of Wall Shear Stress in Carotid Sinus Based on Two-dimensional Color Doppler Imaging,” Biomedical Engineering Online 17(1): 141; van der Palen, R. L. F., Roest, A, A. W., van den Boogaard, P. J., de Roos. A., Blom, N. A., and Westenberg, J. J. M. 2018. “Scan-rescan Reproducibility of Segmental Aortic Wall Shear Stress as Assessed by Phase-specific Segmentation with 4D flow MRI in Healthy Volunteers,” MAGMA [Magnetic Resonance Materials in Physics, Biology, and Medicine] 31(5): 653-663; van Ooij. P., Powell, A, L., Potters, W. V., Carr, J. C., Markl, M., and Barker, A. J. 2016. “Reproducibility and Interobserver Variability of Systolic Blood Flow Velocity and 3D Wall Shear Stress Derived from 4D Flow MRI in the Healthy Aorta,” Journal of Magnetic Resonance Imaging 43(1): 236-248; Soulis, J., Fytanidis, D., Seralidou, K., and Giannoglou, G. 2014. “Wall Shear Stress Oscillation and Its Gradient in the Normal Left Coronary Artery Tree Bifurcations,” Hippokratia 18(1): 12-16; Wang. C., Chen, M., Liu, S, L., Liu, Y., Jin, J. M., and Zhang, Y, H. 2013. “Spatial Distribution of Wall Shear Stress in Common Carotid Artery by Color Doppler Flow Imaging,” Journal of Digital Imaging 26(3): 466-471; Yap, C. H., Saikrishnan, N., and Yoganathan, A. P. 2012. “Experimental Measurement of Dynamic Fluid Shear Stress on the Ventricular Surface of the Aortic Valve Leaflet,” Biomechanics and Modeling in Mechanobiology 11(1-2): 231-244; Efstathopoulos, E. P., Patatoukas, G., Pantos, I., Benekos, O., Katritsis, D., and Kelekis, N. L. 2008. “Measurement of Systolic and Diastolic Arterial Wall Shear Stress in the Ascending Aorta,” Physica Medica (Lugano, Italy) 24(4): 196-203).

FIGS. 12, 13A, and 13B show bidirectional valves. That bidirectional valves do not allow diversion of flow in the one direction to differ from the other is not a drawback, since these do not divert from and to a unidirectional vessel, and the cross-circulatory functions these serve do not call for such differential control. Were differential directional control necessary, unidirectional valves can be made small enough so that two can be used.

Conventionally, once the prospective donor expires, the life support system is shut down and organs for transplantation only then removed. This creates a major complication at the outset, since the loss of perfusion at death initiates significant inflammatory damage, and once reinstated following this ischemia and injury, the normal perfusion of the recipient will further damage the organ, making reperfusion injury a major obstacle to transplantation success. Affecting the transplanted heart in some half of cases, the eventual failure of a heart transplant due to cardiac allograft vasculopathy, even when medically manageable for up to ten years, is believed to be the result of the trauma of death prior to the harvesting of the graft organ.

Reperfusion injury primarily affecting the endothelium, and the endothelium a primary source of inflammation and rejection mediators, the damage is not just pathophysiological but immunological (see, for example, Durigutto, P., Sblattero, D., Biffi, S., De Maso, L., Garrovo, C., and 6 others 2017. “Targeted Delivery of Neutralizing Anti-C5 Antibody to Renal Endothelium Prevents Complement-dependent Tissue Damage,” Online, Frontiers in Immunology (Lausanne, Switzerland) 8:1093; Piotti, G., Palmisano, A., Maggiore, U., Buzio, C. 2014. “Vascular Endothelium as a Target of Immune Response in Renal Transplant Rejection,” Online, Frontiers in Immunology (Lausanne, Switzerland) 5:505:2. Banz, Y. and Rieben, R. 2012. “Role of Complement and Perspectives for Intervention in Ischemia-reperfusion Damage,” Annals of Medicine 44(3): 205-217).

This interruption in perfusion, often lengthy, even involving suffusion in a chilled static or flowing perfusate, results from a lack of the means to allow continuous normal perfusion throughout organ harvesting and repositioning in the circulatory system of the recipient. The problems associated with organ transplantation are immunological—the responses to death, to the loss of perfusion, as well as to confrontation with allogeneic matter. The placement of an automatic disorder response system in the donor, continuation of life support following death, and the use of metered compound vascular bypass, or switched-organ transplantation can considerably reduce if not eliminate these.

In ordinary use where an organ transplant is uninvolved, the object of implanting an automatic adaptive disorder response system is to maintain optimal homeostasis across a potentially fatal condition or a number of independent or interdependent comorbidities. Especially with the advent of this capability, a prospective organ donor should have implanted an automatic adaptive disorder response system controlled by an implanted microprocessor executing a prescription-program which uses data from implanted sensors.

The sensors include various chemical, immune mediator detecting (see, for example, Christiansen, M. P., Klaff, L. J., Brazg. R., Chang, A. R., Levy, C. J., and 7 others 2018. “A PRospective Multicenter Evaluation of the Accuracy of a Novel Implanted Continuous Glucose SEnsor: PRECISE II,” Diabetes Technology and Therapeutics 20(3): 197-206; Koydemir, H, C. and Ozcan, A. 2018. “Wearable and Implantable Sensors for Biomedical Applications,” Annual Review of Analytical Chemistry (Palo Alto California). 11(1): 127-146; Klosterhoff, B. S., Tsang, M., She, D., Ong, K. G., Allen, M. G., Willett, N. J., and Guldberg, R, E. 2017. “Implantable Sensors for Regenerative Medicine,” Journal of Biomechanical Engineering 139(2); Heo. J. C., Kim, B., Kim, Y. N., Kim, D. K., and Lee, J, H. 2017. “Induction of Inflammation In Vivo by Electrocardiogram Sensor Operation Using Wireless Power Transmission,” Sensors (Basel, Switzerland) 17(12). pii: E2905; Ma. Y., Pandzic. E., Nicovich, P. R., Yamamoto, Y., Kwiatek, J., and 4 others 2017. “An Intermolecular FRET [Forster, or florescence, resonance energy transfer] Sensor Detects the Dynamics of T Cell Receptor Clustering,” Nature Communications 8:15100; Yandrapalli, S., Raza, A., Tariq. S., and Aronow, W. S. 2017. “Ambulatory Pulmonary Artery Pressure Monitoring in Advanced Heart Failure Patients,” World Journal of Cardiology 9(1): 21-26; Chassin, H., Geering, B., Schukur, L., Auslander, D., Lang. B., and Fussenegger, M. 2017. “Sensing and Responding to Allergic Response Cytokines through a Genetically Encoded Circuit,” Nature Communications 8(1): 1101; Hewitt, J. 2013. “World's Smallest Blood Monitoring Implant Tells Your Smartphone When You're about to Have a Heart Attack,” ExtremeTech extremetech.com/computing/151134-worlds-smallest-blood-monitoring-implant-talks-to-a-smartphone-but-whose; Cannata, J. M., Chilipka, T., Yang, H. C., Han, S., Ham, S. W., and 4 others 2012. “Development of a Flexible Implantable Sensor for Postoperative Monitoring of Blood Flow,” Journal of Ultrasound in Medicine 31(11): 1795-1802; Lucarelli, F., Ricci, F., Caprio, F., Valgimigli, F., Scuffi, C., Moscone, D., and Palleschi, G. 2012. “GlucoMen Day Continuous Glucose Monitoring System: A Screening for Enzymatic and Electrochemical Interferents,” Journal of Diabetes Science and Technology 6(5): 1172-1181; Merchant, F. M., Dec, G. W., and Singh, J. P. 2010. “Implantable Sensors for Heart Failure,” Circulation. Arrhythmia and Electrophysiology 3(6): 657-667), strain gauge (Kim, J., Lee, H., Cha, S., and Choi, B. 2012. “Fabrication and Evaluation of Implantable Pressure Sensor Using Strain Gauge,” Institute of Electrical and Electronics Engineers Proceedings of the Biomedical Engineering International Conference (BMEiCON), Ubon Ratchathani, Thailand, pages 1-4)-based sensors, and others to include a pulse oximeter, blood gas analyzer, and a cardioverter defibrillator which continuously provide information up through a hierarchy of progressively more comprehensive subordinate node microcontrollers to the overall master node microprocessor.

These signal key events up through the control hierarchy to the master node microprocessor to automatically respond with the release of appropriate agents from implanted reservoirs. Prepared thus both to optimize his own chances for survival but also to have best preserved his organs for the benefit of organ recipients should he die, the prospective donor is kept on life support. Sensors with inmate data generation capability too large to be implanted in situ have only the detection probe implanted and the signal transmitted to its microcontroller, which can be remote

The process is best administered by a master node microprocessor fed alloreactive, immune sufficiency, and blood pressure data through subordinate microcontrollers, the servovalves responding to these indicia as to optimize the immunological conditioning of the recipient and donor organ (see, for example, Beshish, A. G., Bradley, J. D., McDonough, K, L., Halligan, N. L. N., McHugh, W. M., and 4 others 2019, Op cit. under 1. Background of the Invention. a. General; Leentjens, J., Kox, M., Koch, R. M., Preijers, F., Joosten, L. A., and 3 others 2012. “Reversal of Immunoparalysis in Humans in Vivo: A Double-blind, Placebo-controlled, Randomized Pilot Study,” American Journal of Respiratory and Critical Care Medicine 186(9): 838-845). Manual, solenoid, and servomotor valves are shown in FIG. 3. Connection should be from an artery to an artery or a vein into a vein, never by crossing over from an artery to a vein or the reverse, as this would deliver oxygenated blood into unoxygenated blood or the reverse, compromising normal oxygenation and the removal of waste products. Such mischanneling can be thought of as equivalent to birth defects which divert oxygenated blood to the pulmonary artery, for example.

The key difference between repeated draws, infusions, and waiting for laboratory results before proceeding is that the implanted disorder response system accomplishes diagnostics and the response thereto seamlessly, in a continuous stream. Sensor feedback to the prescription-program is instantly translated into the need, rate, and direction of adjustment to the diversion chutes to be directed by the microprocessor. The microprocessor thus has two diagnostic and response tracks—the detection of symptoms with automatic drug response thereto, and control over chute movement. Response in this manner exhibits diagnostic thoroughness and immediacy of response that manual means-taking blood draws and laboratory testing-cannot achieve.

Should the prospective donor die, blood pressure maintenance medication, thyroid hormone, vasopressin, digestive hormones, antimicrobials, and electrolytes, for example, are administered manually or automatically in accordance with the microprocessor prescription-program, and the donor is kept warm. However, using directly pipeline-targeted drug delivery and dynamic sensor input response capability to optimize the administration of therapeutic substances, the implanted automatic response system reacts immediately to best sustain organ viability with a promptness which manual means cannot approach. This capability allows an extension in the time that organs can be kept perfused and transplantable (see, for example, Hoste, P., Hoste, E., Ferdinande. P., Vandewoude, K., Vogelaers, D., and 4 others with 21 collaborators 2018. “Development of Key Interventions and Quality Indicators for the Management of an Adult Potential Donor after Brain Death: A RAND Modified Delphi Approach,” Online, BioMed Central Health Services Research 18(1): 580; Courtwright, A. and Cantu, E. 2017. “Evaluation and Management of the Potential Lung Donor,” Clinics in Chest Medicine 38(4): 751-759; Zens. T. J., Danobeitial. J. S., Chlebeck, P. J., Zitur. L. J., Odorico, S., and 8 others 2017, Op cit.; Pandit, R. A., Zirpe, K. G., Gurav, S. K., Kulkami, A. P., Karnath, S., and 10 others 2017. “Management of Potential Organ Donor: Indian Society of Critical Care Medicine: Position Statement,” Indian Journal of Critical Care Medicine 21(5): 303-316; Mundt, H. M., Yard, B. A., Krämer, B. K., Benck, U., and Schnulle, P. 2016. “Optimized Donor Management and Organ Preservation before Kidney Transplantation,” Transplantation International 29(9): 974-984; Kumar, L. 2016, Op cit.; Kotloff, R. M., Blosser, S., Fulda, G., Malinoski, D., Ahya, V. N., and 37 others 2015. “Management of the Potential Organ Donor in the ICU: Society of Critical Care Medicine/American College of Chest Physicians/Association of Organ Procurement Organizations Consensus Statement,” Critical Care Medicine (6): 1291-1325; Danobeitia, J. S., Hanson, M. S., Chlebeck, P., Park, E., Sperger, J. M., Schwarznau, A., and Fernandez, L, A. 2015. “Donor Pretreatment with IL-1 Receptor Antagonist Attenuates Inflammation and Improves Functional Potency in Islets from Brain-dead Nonhuman Primates,” Cell Transplantation 24(9): 1863-1877; Dhanani, S. and Shemie, S, D. 2014. “Advancing the Science of Organ Donor Management,” Critical Care 18(6): 612; Valenza, F., Coppola, S., Froio, S., Ruggeri, G. M., Fumagalli, J., and 10 others 2014. “A Standardized Model of Brain Death, Donor Treatment, and Lung Transplantation for Studies on Organ Preservation and Reconditioning,” Intensive Care Medicine Experimental 2(1): 12; Murthy, T. 2009. “Organ Donation: Intensive Care Issues in Managing Brain dead,” Medical Journal, Armed Forces India 65(2): 155-160). Unpreventable using conventional means, the considerable injury added to that of death by stopping life support and excising the graft organ is limited to amelioration after the fact.

However, by redirecting the circulation of the recipient from his own diseased organ to and through the corresponding organ of the donor before either organ is removed, vascular servovalves driven on the basis of data input by strategically positioned sensors (see, for example. Christiansen, M. P., Klaff, L. J., Brazg, R., Chang, A. R., Levy, C. J., and 7 others 2018. “A PRospective Multicenter Evaluation of the Accuracy of a Novel Implanted Continuous Glucose SEnsor: PRECISE II,” Diabetes Technology and Therapeutics 20(3): 197-206; Koydemir, H, C. and Ozcan, A. 2018. “Wearable and Implantable Sensors for Biomedical Applications,” Annual Review of Analytical Chemistry (Palo Alto California). 11(1): 127-146; Lorenz, C., Sandoval, W., and Mortellaro, M. 2018. “Interference Assessment of Various Endogenous and Exogenous Substances on the Performance of the Eversense Long-Term Implantable Continuous Glucose Monitoring System,” Diabetes Technology and Therapeutics 20(5): 344-352; Afari, M. E., Syed. W., and Tsao. L. 2018. “Implantable Devices for Heart Failure Monitoring and Therapy,” Heart Failure Reviews [in press]; De Rosa, R., Piscione, F., Schranz, D., Citro, R., lesu S., and Galasso, G. 2018. “Transcatheter Implantable Devices to Monitoring of Elevated Left Atrial Pressures in Patients with Chronic Heart Failure,” Translational Medicine at UniSa [University of Salerno] 17:19-21; Drazan, J. F., Abdoun, O. T., Wassick, M. T., Dahle, R., Beardslee, L., Marcus, G. A., Cady, N. C., and Ledet, E. H. 2018. “Simple Implantable Wireless Sensor Platform to Measure Pressure and Force,” Medical Engineering and Physics 59:81-87; Kamišalić, A., Fister, I. Jr., Turkanović, M., and Karakatič, S. 2018. “Sensors and Functionalities of Non-invasive Wrist-wearable Devices: A Review,” Sensors (Basel, Switzerland) 18(6). pii: E1714; Tschope. C., Alogna, A., Spillmann, F., Faragli, A., Schmidt, G., and 9 others 2018. “The CardioMEMS System in the Clinical Management of End-stage Heart Failure Patients: Three Case Reports,” Online, BioMed Central Cardiovasc Disorders 18(1): 155; Klosterhoff, B. S., Tsang, M., She, D., Ong, K. G., Allen, M. G., Willett, N. J., and Guldberg, R, E. 2017. “Implantable Sensors for Regenerative Medicine,” Journal of Biomechanical Engineering 139(2): Wang, J. T. and Frishman, W, H. 2017. “Pulmonary Pressure Monitoring for Patients with Heart Failure,” Cardiology in Review 25(2): 53-58; Givertz, M. M., Stevenson, L. W., Costanzo, M. R., Bourge, R. C., Bauman, J. G., Ginn, G., and Abraham W. T. 2017. “Pulmonary Artery Pressure-guided Management of Patients with Heart Failure and Reduced Ejection Fraction,” Journal of the American College of Cardiology 70(15): 1875-1886; Heywood, J. T., Jermyn, R., Shavelle, D., Abraham, W. T., Bhimaraj. A., and 8 others 2017. “Impact of Practice-based Management of Pulmonary Artery Pressures in 2000 Patients Implanted with the CardioMEMS Sensor,” Circulation 135(16): 1509-1517; Heo. J. C., Kim, B., Kim, Y. N., Kim, D. K., and Lee, J. H. 2017. “Induction of Inflammation In Vivo by Electrocardiogram Sensor Operation Using Wireless Power Transmission,” Sensors (Basel, Switzerland) 17(12). pii: E2905; Ma, Y., Pandzic, E., Nicovich, P. R., Yamamoto, Y., Kwiatek, J., and 4 others 2017. Op cit. above under (b). Metered compound vascular bypass, or sudden switched-organ transplantation; Yandrapalli, S., Raza, A., Tariq, S., and Aronow, W. S. 2017. “Ambulatory Pulmonary Artery Pressure Monitoring in Advanced Heart Failure Patients,” World Journal of Cardiology 9(1): 21-26; Chassin, H., Geering, B., Schukur, L., Auslander, D., Lang. B., and Fussenegger, M. 2017. “Sensing and Responding to Allergic Response Cytokines through a Genetically Encoded Circuit,” Nature Communications 8(1): 1101; Yu, L., Kim, B. J., and Meng, E. 2014. “Chronically Implanted Pressure Sensors: Challenges and State of the field,” Sensors (Basel, Switzerland) 14(11): 20620-20644; Hewitt, J. 2013. Op cit., above in this section; Inmann A. and and Hodgins, D. (eds.) 2013. Implantable Sensor Systems for Medical Applications, Woodland Publishing: Lucarelli, F., Ricci, F., Caprio, F., Valgimigli, F., Scuffi, C., Moscone, D., and Palleschi, G. 2012. “GlucoMen Day Continuous Glucose Monitoring System: A Screening for Enzymatic and Electrochemical Interferents,” Journal of Diabetes Science and Technology 6(5): 1172-1181; Cannata, J. M., Chilipka, T., Yang, H. C., Han, S., Ham, S. W., and 4 others 2012. “Development of a Flexible Implantable Sensor for Postoperative Monitoring of Blood Flow,” Journal of Ultrasound in Medicine 31(11): 1795-1802; Kim, J., Lee, H., Cha, S., and Choi, B. 2012. “Fabrication and Evaluation of Implantable Pressure Sensor Using Strain Gauge,” Institute of Electrical and Electronics Engineers Proceedings of the Biomedical Engineering International Conference (BMEiCON), Ubon Ratchathani, Thailand pages 1-4; Merchant, F. M., Dec, G. W., and Singh, J. P. 2010. “Implantable Sensors for Heart Failure,” Circulation. Arrhythmia and Electrophysiology 3(6): 657-667) feeding data to an automatic response system make it possible to harvest and transplant any solid organ without interruption in perfusion (FIG. 1). Vascular servovalves can be extended or retracted incrementally or continuously at any rate in direct response to the pathophysiological indicia reported to and processed by the microprocessor in accordance with the prescription-program.

Sudden compound vascular bypass, or sudden switched-organ transplantation is suitable when the prospective recipient is nearing death due to end-stage organ failure and/or a deceased organ donor, notwithstanding having been maintained on life support, has deteriorated to a degree that allows only so much time for transplantation. The sudden technique is therefore responsive to an urgency that demands immediate action, but does so at the cost of precluding measured sensor-responsive advancement of the diversion chutes, and therewith, the suppressive immunotherapeutic blending of donor and recipient blood at a controlled rate as best achieves the induction of immune tolerance; however, such medication can be administered to the patient manually both mid- and postprocedurally (see, for example, van Zanden, J. E., Jager, N. M., Daha, M. R., Erasmus, M. E., Leuvenink, H, G. D., and Seelen, M, A. 2019. Op cit. above under 1. Background of the Invention. a. General: Ueta, H., Kitazawa, Y., Sawanobori, Y., Ueno, T., Ueha, S., Matsushima, K., and Matsuno, K. 2018. Op cit.; Moncharmont. P. 2018. “Platelet Component Transfusion and Alloimmunization: Where Do We Stand?,” Transfusion Clinique et Biologique 25(3): 172-178; Durigutto, P., Sblattero. D., Biffi. S., De Maso, L., Garrovo. C., and 6 others 2017. “Targeted Delivery of Neutralizing Anti-C5 Antibody to Renal Endothelium Prevents Complement-dependent Tissue Damage,” Online, Frontiers in Immunology (Lausanne, Switzerland) 8:1093; Youssef. L, A. and Spitalnik, S. L. 2017. “Transfusion-related Immunomodulation: A Reappraisal,” Current Opinion in Hematology 24(6): 551-557; Jovičić, S., Ležaić, V., Simonović, R., and Djukanović, L. 2011. “Beneficial Effects of Donor-specific Transfusion on Renal Allograft Outcome,” Clinical Transplantation 25(2): 317-324).

By contrast, unless weakly anti-type A or anti-type B, an adult recipient is relatively ABO mismatch-intolerant (Tyden, G.; Hagerman, I.; Grinnemo, K. H.; Svenarud, P.; van der Linden, J. Kumlien, G; and Wernerson, A. 2012. “Intentional ABO-incompatible Heart Transplantation: A Case Report of 2 Adult Patients,” Journal of Heart and Lung Transplantation (New York, New York) 31 (12): 1307-1310). In sudden compound vascular bypass, or sudden switched-organ transplantation, tolerance induction is achieved by preprocedural manual reciprocal cross infusion between donor and recipient.

In metered compound vascular bypass, or sudden switched-organ transplantation, tolerance induction can proceed during the transit of the vascular valve diversion chutes which gradually transfer the graft organ from the donor to the recipient. The paper cited below by Wang, et al. 2020 discusses a means for the induction of immune tolerance which does not render the patient more susceptible to infection (Lin, G., Wang, J., Yang, Y. G., Zhang. Y., and Sun, T. 2023. “Advances in Dendritic Cell Targeting Nano-delivery Systems for Induction of Immune Tolerance,” Online, Frontiers in Bioengineering and Biotechnology (Lausanne, Switzerland) 11:1242126; Rosenberg, A. S., Tourdot, S., and Markusic, D. 2023. “Editorial: Induction of Immune Tolerance: Addressing Unmet Medical Need in Immune Mediated diseases and Immune Responses to Biologics,” Online, Frontiers in Immunology (Lausanne, Switzerland) 14:1219854; Li, H., Yang, Y. G., and Sun, T. 2022. “Nanoparticle-based Drug Delivery Systems for Induction of Tolerance and Treatment of Autoimmune Diseases,” Online, Frontiers in Bioengineering and Biotechnology (Lausanne, Switzerland) 10:889291; Nepom, G. T. 2022. “Synergistic Targeting of Immunologie Pathways to Empower Durable Tolerance Therapies,” Online, Frontiers in Immunology (Lausanne, Switzerland) 13:962177; Castenmiller. C., Keumatio-Doungtsop, B. C., van Ree. R., de Jong, E. C., and van Kooyk, Y. 2021. “Tolerogenic Immunotherapy: Targeting DC [dendritic cells] Surface Receptors to Induce Antigen-specific Tolerance,” Online, Frontiers in Immunology (Lausanne, Switzerland) 12:643240; Moorman, C. D., Sohn, S. J., and Phee, H. 2021. “Emerging Therapeutics for Immune Tolerance: Tolerogenic Vaccines. T Cell Therapy, and IL-2 Therapy,” Online, Frontiers in Immunology (Lausanne, Switzerland) 12:657768; Bluestone, J, A. and Anderson, M. 2020. “Tolerance in the Age of Immunotherapy,” Online, New England Journal of Medicine (Boston, Massachusetts) 383(12): 1156-1166; Svaiger, U. and Rozman, P. J. 2020. “Recent Discoveries in Dendritic Cell Tolerance-inducing Pharmacological Molecules,” International Immunopharmacology (Amsterdam, North Holland, Netherlands) 81:106275; Wang, Z., Liu, X., Cao, F., Bellanti, J. A., Zhou, J., and Zheng. S, G. 2020. “Prospects of the Use of Cell Therapy to Induce Immune Tolerance,” Online, Frontiers in Immunology (Lausanne, Switzerland) 11:792; Parks, R. J. and Gussoni, E. 2018. “Building Immune Tolerance through DNA Vaccination,” Online, Proceedings of the National Academy of Sciences of the United States of America (Washington, D.C.) 115(39): 9652-9654; Ferrera, F., La Cava, A., Rizzi, M., Hahn, B. H., Indiveri, F., and Filaci, G. 2007. “Gene Vaccination for the Induction of Immune Tolerance,” Annals of the New York Academy of Sciences (New York, New York) 1110:99-111; Rotrosen, D., Matthews, J. B., and Bluestone, J, A. 2002. “The Immune Tolerance Network: A New Paradigm for Developing Tolerance-inducing Therapies,” Online, Journal of Allergy and Clinical Immunology (St. Louis, Missouri) 110(1): 17-23).

The period during which a donor can receive an ABO incompatible transplant can be extended by intentional exposure to non-self A and B blood type antigens (Fan, X., Ang, A., Pollock-Barziv. S. M., Dipchand, A. I., Ruiz, P., and 3 others 2004. “Donor-specific B-cell Tolerance after ABO-incompatible Infant Heart Transplantation,” Nature Medicine 10(11): 1227-1233). Moreover, in young children with less developed immune systems, having been effectively tolerance induced by previous exposure to a type AB graft, the B type recipient will then be tolerant to type AB graft organs should the same organ have to be retransplanted or another organ transplanted.

Rituximab-targeted B-cell depletion combined with antibody removal by immunoadsorption or plasmapheresis appear to diminish blood type intolerance to graft kidneys, lungs, and livers in adults more than to graft hearts (see, for example, Lee, J., Kim, D. G., Kim, B. S., Kim, M. S., Il Kim, S., Kim, Y. S., and Huh, K, H. 2019. “Early Hospital Readmissions after ABO- or HLA-incompatible Living Donor Kidney Transplantation,” (Nature Research) Scientific Reports 9(1): 3246; de Weerd, A, E. and Betjes, M, G, H. 2018. “ABO-incompatible Kidney Transplant Outcomes: A Meta- Analysis,” Clinical Journal of the American Society of Nephrology 13(8): 1234-1243; Nishimura, H., Yamada, Y., Hisano, S., Mitsuke, A., Tatarano, S., and 4 others 2018, Op cit. above under 1. Background of the Invention a. General, Locke, J, E. 2018. “Expand the Pool of Living Donors for Kidney Transplantation,” Clinical Journal of the American Society of Nephrology 13(8): 1142-1143; Ko, E. J., Yu, J. H., Yang, C. W., and Chung, B. H., with 14 collaborators 2017. “Clinical Outcomes of ABO- and HLA-incompatible Kidney Transplantation: A Nationwide Cohort Study,” Transplant International 30(12): 1215-1225; Chen, G., Sun, J., Wei. S., Chen, Y., Tang. G., and 9 others 2017. “Simultaneous ABO-incompatible Living-donor Liver Transplantation and Splenectomy without Plasma Exchange in China: Two case Reports,” Journal of International Medical Research 45(6): 2146-2152; Urschel. S. and West. L. J. 2016. “ABO-incompatible Heart Transplantation,” Current Opinion in Pediatrics 28(5): 613-619; Ikegami, T., Yoshizumi, T., Soejima, Y., Uchiyama, H., Shirabe, K., and Maehara, Y. 2016. “Feasible Usage of ABO Incompatible Grafts in Living Donor Liver Transplantation,” Hepatobiliary Surgery and Nutrition 5(2): 91-97; Opelz, G., Morath, C., Susal, C., Tran, T. H., Zeier, M., and Dohler, B. 2015. “Three-year Outcomes following 1420 ABO-incompatible Living-donor Kidney Transplants Performed after ABO Antibody Reduction: Results from 101 Centers,” Transplantation 99(2): 400-404; Lee, S. D., Kim, S. H., Kong, S. Y., Kim, Y. K., Lee, S. A., and Park, S. J. 2014. “ABO-incompatible Living Donor Liver Transplantation without Graft Local Infusion and Splenectomy,” Hepato-Pancreato-Biliary (Oxford, England) 16(9): 807-813; Urschel, S.; Larsen, I/M/; Kirk, R.; Flett, J/; Burch, M.; and 9 others 2013. “ABO-incompatible Heart Transplantation in Early Childhood: An International Multicenter Study of Clinical Experiences and Limits,” The Journal of Heart and Lung Transplantation (New York, New York) 32(3): 285-292; Soejima, Y., Muto, J., Matono, R., Ninomiya, M., Ikeda, T., and 5 others 2013. “Strategic Breakthrough in Adult ABO-incompatible Living Donor Liver Transplantation: Preliminary Results of Consecutive Seven Cases,” Clinical Transplantation 27(2): 227-231; West, J. L. 2011, Op cit. under 1. Background of the invention. a. General; Saczkowski, R., Dacey, C., and Bernier, P. L. 2010. Does ABO-incompatible and ABO-compatible Neonatal Heart Transplant Have Equivalent Survival?,” Interactive Cardiovascular and Thoracic Surgery 10(6): 1026-1033).

Where sudden compound vascular bypass, or sudden switched-organ transplantation abruptly resituates the graft organ from the circulatory system of the donor into that of the recipient, necessitating manual blood draws and extracorporeal donor-recipient cross transfusion for tolerance induction and injection or infusion of immunosuppressants and other drugs to control if not avoid adverse alloreactions, metered rather than sudden advancement of the vascular valve diversion chutes allows the gradual inversely proportional, or complementary, intermixture at the same rate of exposure of each to the blood of the other.

Such includes reciprocal tolerance-inducing agents between donor and recipient, merging physical or anatomical transplantation and tolerance induction into a single operation. That is, where sudden compound vascular bypass, or sudden switched-organ transplantation uses bistable solenoid-based valves to accomplish physical transplantation, the use of servovalves both optimizes and merges the reconciliation of the donor and recipient using medical support in a single or merged operation.

This gradually, rather than abruptly, transfers the donor organ to the circulatory system of the recipient in a manner that affords gradual rather than abrupt exposure to the allogeneic antigens of one another, the degree of acclimatization or adverse reaction proportional to the interval and volume of blood exchange over which the process continues until the donor is closed off from with the donor organ fully incorporated into the recipient. When automatically monitored, immunological resistance is detected instantly, as is the application of remedial measures by the prescription-program to include adjustment or sudden full extension of the diversion chutes and the targeted release of drugs through the valve accessory channels.

Moreover, by allowing controlled advancement rather than a sudden switching from the fully closed (chute retracted, non-diverting) to the fully open (chute extended, diverting) position of the valve blood diversion chutes, the system can so govern the apportionment of blood volume between recipient and donor as to optimize the endogenous, or drugless, induction of graft organ-recipient immune tolerance while the graft organ is gradually switched from the circulatory system of the donor to that of the recipient allowing any adverse alloreaction which drugs can reverse to be automatically released instantly.

Otherwise, the sudden loss of blood with its anoxia, loss of endothelium-derived vasoactive factors, hormones, blood electrolytes, enzymes, and immune factors serves as the pathophysiological trigger for the cascade of inflammation which seriously impairs the graft organ for transplantation. Allowing the delivery of drugs to the donor prior to brain death without subsequent interruption in cardiopulmonary function following brain death, both a sudden and metered compound vascular bypass, or sudden switched-organ transplantation avert much of the inflammatory degradation and immune arousal which ensue upon brain death. Left to proceed without intervention prior to transplantation, these jeopardize the success of transplantation due not only to reperfusion injury but an increase in immunological discord beyond that ordinarily encountered in addition to reperfusion injury.

Whether sudden or metered, switch transplantation is accomplished in much less time and with less dissection than is conventional transplantation, reducing time under anesthesia and trauma, and expanding the scope of patients able to undergo surgery. In end-stage heart disease, this last factor is significant. The procedural duration of metered compound vascular bypass, or sudden switched-organ transplantation depends upon the immunological disparity between recipient and donor.

Where this is more pronounced and exogenous agents and blood products are not used, procedural time may exceed that conventional. However, this time is not used for dissection but rather for inducing immune tolerance between recipient and donor. With either sudden or metered transplantation, surgery consists of entry, placing the diversion valves, and closing, after which local anesthesia can be directly targeted through the accessory channels and if placed, electrostimulators.

Trauma is less, and the range of surveillance and immediacy of automatic disorder system response can serve to suppress numerous complications. Under conditions of uninterrupted perfusion, the increased immune response of the donor attendant upon brain death may be ameliorated or require the infusion of immunosuppressants in excess compared to that needed with a living donor. In metered compound vascular bypass, or sudden switched-organ transplantation, unless countermeasures are applied to remove objectionable substances such as an anesthetic, drugs, and immune mediators associated with brain injury and death from the blood of the donor and/or recipient, these will flow from the one into the other.

Such counteractive measures, which should be instituted before the procedure is initiated, include reversal, dissolution, or neutralization through bonding with a disabling substituent, for example, include the use of drugs, plasmapheresis, plasma exchange, the use of natural, or intrinsic, counteractives, and bonding with superparamagnetic micro- or nanoparticles for magnetic extraction. A ‘two-edged sword,’ the exchange of allogeneic material between donor and recipient either arouses adverse alloreactions or succeeds in inducing some measure of endogenous tolerance for the transplant to follow. For some reactions, the alien material will ‘ignore,’ and not provoke a reaction until a certain burden accrues.

A gradual metering rather than an abrupt exchange or reciprocal infusion of blood between recipient and donor, especially when precisely monitored and able to automatically detect the need for and deliver anti-alloreactive agents, is, however, less likely to provoke an adverse reaction. A coordinated ability to gradually meter the delivery of immunosuppressives, tolerance-inducing, cross-chimerizing, and vascular maintenance substances as the graft organ is gradually transferred from the deceased’ donor to the recipient continuously optimizes the assimilation of and tolerance for the transplant.

As defined above in section 2a, the term ‘transfusion’ denotes the administration of blood of blood products taken from another delivered into a vein through a hypodermic needle. ‘Reciprocal of cross transfusion’ then, denotes this interrupted manual means for exchanging blood of blood products between donor and recipient. The term ‘infusion’ pertains to drugs rather than blood or blood products administered in the same way, ruling out use of the terms ‘reciprocal or cross infusion. ‘Cross-circulation’ denotes a direct exchange of blood through a continuous circuit connecting a donor and a recipient.

Organs from deceased donors, especially when preceded by severe brain injury (see, for example, Watts, R. P., Thom, O., and Fraser, J, F. 2013. “Inflammatory Signalling Associated with Brain dead Organ Donation: from Brain Injury to Brain Stem Death and Posttransplant Ischaemia Reperfusion Injury,” Journal of Transplantation 2013:521369), have proven markedly inferior to those from living donors-important, since a patient with end-stage congestive heart failure needs a replacement heart, which unpaired, cannot be provided by a living donor.

The substantial difference between a deceased and living donor organ the fact of having been preceded by death, it is to this event which may be ascribed the greatest injury. Preferably, prior to the terminal event, the donor will have been provided with an automatic disorder response system to assist in his own survival, but also to protect the graft organ or organs by preventing inflammation or vasospasm which could obstruct the flow of blood should he die. Sudden and metered switch valves both make possible the harvesting of the graft organ before removal and without interruption in perfusion.

Not involving extended reciprocal cross-circulation, the sudden compound vascular bypass, or sudden switched-organ technique suddenly isolates the graft organ for the flowing through of recipient blood with no recipient blood flow through the rest of the donor circulatory system or the reverse. If reciprocal cross-circulation is not sustained, the only blood from the donor which is passed to the recipient is that flushed out of the graft organ, making the load of objectionable material of nugatory pertinence, especially since the presence thereof would have been identified and disregarded or neutralized by preprocedural infusion if not rejection of the donor.

In contrast, using the metered compound vascular bypass, or sudden switched-organ technique, reciprocal cross-circulation continues so long as the vascular servovalve diversion chutes are at any position other than fully retracted, closing off the circulation of either participant from that of the other. The blood of the recipient is therefore not limited to the graft organ, but in order to sweep over the endothelium of both donor and recipient and exchange immune mediators is intentionally circulated throughout the circulatory systems of both for the purpose of reciprocal immune tolerance induction.

This in itself can provoke adverse alloreactions in the recipient. Moreover, anything in the blood of a live donor, to include an anesthetic, drugs, and possibly pathogens, or anything in the blood of a deceased donor, to include the innate and adaptive immune system mediators associated with death as well, will enter the circulation of the recipient or recipients and their merged circulation. Any residual drugs and the immune mediators attendant upon death will continue to flow into the recipient at the instant rate and volume of flow set by the valves from the donor to the recipient. It is therefore important to identify such substances and preferably, neutralize these manually in advance, or if the potential for these to prove problematic is indeterminable, furnish the automatic system with broad spectrum cytokine reversal or moderating agents.

While the mediators attendant upon death will have been diminished by the preservation of oxygenation and perfusion afforded by the extracorporeal membrane oxygenator past the terminal event, the inducement of adverse sequelae in the graft and distant potential graft organs must be identified and ameliorative measures, such as the administration to the donor of type 2 cytokines/interleukin instituted (see, for example, Watts, R. P., Thom, O., and Fraser, J, F. 2013. “Inflammatory Signalling Associated with Brain dead Organ Donation: from Brain Injury to Brain Stem Death and Posttransplant Ischaemia Reperfusion Injury,” Journal of Transplantation 2013:521369; Pandit, R. A., Zirpe, K. G., Gurav, S. K., Kulkami, A. P., Kamath, S., and 10 others 2017. “Management of Potential Organ Donor: Indian Society of Critical Care Medicine: Position Statement,” Indian Journal of Critical Care Medicine 21(5): 303-316).

In metered compound vascular bypass, or sudden switched-organ transplantation, vascular valves not only make possible transplantation without interruption in perfusion of the graft organ, but position the graft organ in both the donor and recipient circulatory systems to initiate immune tolerance where the interval over which this mutual exposure continues is tightly controllable. In mass casualty circumstances, reciprocal cross-circulation is not limited to two patients in need of partial liver or lung transplants, for example.

In metered compound vascular bypass solid organ transplantation, a progressively increased fraction of the blood supplied to the graft organ is replaced with that of the recipient or recipients, gradually introducing immune mediating factors distinctive of the recipient or recipients. The disorder response system that administered the transplant sustains it through the detection of the need for and dispensing of counteractive agents responsive to the appearance of rejection, infection, malignancy, metabolic disorders, and chronic graft organ disease, (see, for example, Bhat, M., Pasini, E., Patel, P., Yu, J., Baciu, C., Kurian, S. M., and Levitsky, J. 2023. “Achieving Tolerance Modifies Cancer Susceptibility Profiles in Liver Transplant Recipients,” Online, Cancer Medicine (Malden, Massachusetts) 12(4): 5150-5157; Gille, I., Claas, F, H. J., Haasnoot, G. W., Heemskerk, M, H. M., and Heidt, S. 2022. “Chimeric Antigen Receptor (CAR) Regulatory T-cells in Solid Organ Transplantation,” Online, Frontiers in Immunology (Lausanne, Switzerland) 13:874157; Cvetkovski, F., Hexham, J. M., and Berglund, E. 2021. “Strategies for Liver Transplantation Tolerance,” Online, MDPI [Multidisciplinary Digital Publishing Institute] International Journal of Molecular Sciences (Basel, Switzerland) 22(5): 2253; Levitsky, J. and Feng, S. 2018. “Tolerance in Clinical Liver Transplantation,” Human Immunology (New York, New York) 79(5): 283-287; Ueta, H., Kitazawa, Y., Sawanobori, Y., Ueno, T., Ueha, S., Matsushima, K., and Matsuno, K. 2018, Op cit., section 2a; Utter, Dangi, A., Sumpter, T, L., Kimura, S., Stolz, D. B., Murase, N., and 5 others 2012. “Selective Expansion of Allogeneic Regulatory T Cells by Hepatic Stellate Cells: Role of Endotoxin and Implications for Allograft Tolerance,” Online, Journal of Immunology (Baltimore, Maryland) 188(8): 3667-3677; G. H., Owings, J. T., Lee, T. H., Paglieroni, T. G., Reed, W. F., Gosselin, R. C., Holland, P. V., and Busch, M. P. 2004. “Blood Transfusion is Associated with Donor Leukocyte Microchimerism in Trauma Patients,” Journal of Trauma 57(4): 702-708; Wood, K. J. 2003. “Passenger Leukocytes and Microchimerism: What Role in Tolerance Induction?,” Transplantation 75(9 Supplement): 17S-20S; Weber, S., Salguero, R., Allers, C., Blaheta, R. A., and Markus, B, H. 2003. “Amount of Co-transplanted Donor-derived Leukocytes Determines In-vivo Microchimerism and Mixed Lymphocyte Culture Changes Post-liver Transplantation,” Zentralblatt für Chirurgie (Stuttgart, Germany) 128(4): 278-282.

The disorder response system can be programmed to switch to another remedial agent when necessary (see, for example, Giral, M., Grimbert, P., Morin, B., Bouvier, N., Buchler, M., and 8 others 2024. “Impact of Switching from Immediate- or Prolonged-release to Once-daily Extended-release Tacrolimus (LCPT) [LifeCycle Pharma Tacrolimus] on Tremor in Stable Kidney Transplant Recipients: The Observational ELIT [Evolution a Long-terme des tremblements latrogenes de Tacrolimus, or Long-term Outcomes of Tacrolimus-induced Tremor] Study,” Online, Transplant International (Lausanne, Switzerland) 37:11571; Tholking, G., Tosun-Koc, F., Jehn, U., Koch, R., Pavenstadt, H., Suwelack, B., and Reuter, S. 2022. “Improved Kidney Allograft Function after Early Conversion of Fast IR-Tac [immediate release tacrolimus] Metabolizers to LCP-Tac [LifeCycle Pharma Tacrolimus],” Online, MDPI [Multidisciplinary Digital Publishing Institute] International Journal of Clinical Medicine (Basel, Switzerland) 11(5): 1290; Tholking, G., Gillhaus, N. H., Schutte-Nutgen, K., Pavenstadt, H., Koch, R., Suwelack, B., and Reuter, S. 2020. “Conversion to Everolimus was Beneficial and Safe for Fast and Slow Tacrolimus Metabolizers after Renal Transplantation,” Online, MDPI [Multidisciplinary Digital Publishing Institute] International Journal of Clinical Medicine (Basel, Switzerland) 9(2): 328; Husing, A., Schmidt, M., Beckebaum, S., Cicinnati, V. R., Koch, R., and 5 others 2015. “Long-term Renal Function in Liver Transplant Recipients after Conversion from Calcineurin Inhibitors to mTOR [mammalian target of rapamycin] Inhibitors,” Online, Annals of Transplantation (Smithtown, New York) 20:707-713).

When more than one recipient is connected to a single donor, the bloodstreams blend to perfuse all their organs. When the valves are first advanced, recipient and donor exchange the fraction of blood corresponding to the extent of the advancement, which progressively increased at the rate established, becomes dispersed throughout the body of the other or others, and pointedly, through both of their spleens, lymph nodes, regional lymphatics to include Waldeyer's ring (tonsils, adenoids), Peyer's patches, and the lymph nodes, thymus glands, and bone marrow. Shared circulation peaks in the central of the views shown in FIGS. X and X, where the blood of both donor and recipient pass through the valve. At the same time, the accessory channels, as well as conventional infusion, can be used to supplement the blood products exchanged, and if exceptionally, small sections of various organs transplanted manually.

Cross-circulation in the sense intended should not be misconstrued as the unilateral support of a smaller patient by a larger volunteer whose heart is used to support the patient as well as himself with the aid of a pump positioned along the line connecting the two. The term ‘reciprocal cross-circulation’ is used to distinguish the unilateral form of cross-circulation used by C. Walton Lillehei to allow open heart surgery from the bilateral form meant here. Thus, where the term ‘cross-circulation’ denotes unilateral supply with substantially passive return, ‘reciprocal cross-circulation’ as used here denotes two-way active exchange with both hearts pumping.

Whereas vascular servovalves can meter the extent of redirection in blood flow by guarded degrees during which induction tolerance through gradually increased donor blood flow and anti-inflammatory drugs proceeds with any adverse alloreactions detected and reversal measures instituted instantly, sudden switch valves redirect the circulation of the recipient from his own to the replacement organ and donor quickly and therefore preempt a period for reciprocal cross-circulation to allow endogenous immune induction. To assure the synchronous action of all valves, sudden compound vascular bypass, or sudden switched-organ transplantation necessitates at least damped non-sparking plunger solenoid-based valves or motor-driven servovalves, while metered compound vascular bypass, or sudden switched-organ transplantation necessitates continuously adjustable servovalves.

If for any reason such as exigency the applicability of the sudden switch technique cannot be resolved in advance, the use of motor-driven servovalves allows the choice of method midprocedurally. That is, whereas solenoid-driven valves are incapable of gradual action, servomotor-driven valves are. The process is initiated by implanting a prosthetic disorder response system in a donor on life support to bolster his own chances for recovery, the system then having been prepositioned to detect and react to the signs of death immediately. With a living donor, only the recipient has such a system implanted. Outflow and inflow valve pairs on corresponding vessels of both donor and recipient moving in synchrony allows control over the relative proportion of cross inflow and outflow through the supply and drainage vessels of the graft organ, donor, and recipient.

Valve opening, hence, the volume of reciprocal cross-circulation, responds to sensor data supplied to an implanted control microprocessor able not only to continuously adjust the valves but command the release of drugs through accessory channels in the valves in accordance with its prescription-program. This gradual creep in increased relative proportion of allogeneic material is intended to allow time for immune system assimilation and stabilization. The system is therefore able to respond to the effects of immunological incompatibility and any other complications the instant such is detected, thus allowing the acclimatization to proceed without significant adverse events.

The deceased donor having been started on life support before dying, the overall consequence is that the use of vascular servovalves confers the capability to merge, in concurrence and equivalent proportion, tolerance induction and transplantation as do bistable valves but with the transition from circulatory isolation to full exchange extended rather than abrupt. Because the system mollifies the immediate consequences of death while on life support with an efficiency that manual blood draws, laboratory testing, and finally administration of the required reversal agents or replacement of essential substances cannot approach, it is most valuable with a deceased donor whose organs would otherwise be inferior to those of a living donor.

With a kidney or lung from a living donor, the value of the system lies in providing continuous perfusion throughout transplantation with control over the rate and interval over which the blood of the participants is blended. Such averts much of the injury associated with graft organ excision, the temporary loss of perfusion, and an adverse immune reaction to sudden rather than a concurrently and gradually mitigated confrontation with allogeneic matter. The first insult is death, the second is the shutting off of life support with the loss of perfusion, nutrients, and other essential substances, the third is the shock of excision, the fourth when applicable, storage, the fifth reperfusion injury, and the sixth immune intolerance. The use of vascular servovalves as the effectors responsive to an implanted automatic response system efficiently mollifies the inflammation of death and eliminates the stopping of perfusion that would otherwise provoke a secondary ‘cytokine storm.’

When excised, the graft organ has already been switched to the circulatory system of the recipient, has never ceased to be perfused, and with an immunologically significant percent of the recipient's blood. An invasive procedure such as heart transplantation imposing considerable trauma just by virtue of its invasiveness, a material reduction in other sources of transplantation injury improves the odds for a successful outcome, especially in light of the debilitated condition of patients with end-stage heart failure. Organ transplantation using the method described is without incision into the graft organ. Such a heart transplant, for example, is extracardiac, eliminating much of the trauma associated with conventional heart transplantation.

The delivery of drugs is through the valve-jacket accessory channel or channels (FIG. 2). Such release is executed and monitored by the control microprocessor on the basis of feedback from pathophysiological sensors in accordance with its prescription-program. At the outset, the implanted sensors provide initial and then start of transfer status of donor, recipient, and donor organ to be transplanted, establishing the term over which metered transplantation may continue to achieve optimized tolerance induction. One deceased donor can provide organs to more than one recipient. Due to donor deterioration, this is best done simultaneously rather than successively. Either way, using metered compound vascular bypass, or sudden switched-organ transplantation, all three will be tolerance induced or cross-microchimerize for the other two.

Both sudden and metered compound vascular bypass, or sudden switched-organ transplantation not only accommodate the use of immunosuppressants, plasmapheresis, plasma exchange, antimicrobials, and collateral surgical measures such as splenectomy, for example, but implement the optimized use thereof, detecting the need therefor and immediately dispensing required substances. Valve pairs consist of outflow and inflow valves in either party respectively connected to an inflow and outflow valve in the other. Each arm of the reciprocal cross-circulation circuit thus comprises an outflow and an inflow valve connected by a mainline, flow from the outflow to the inflow valve of either party connecting his own circulatory system to that of the other constituting the arms which complete the circuit.

Due to the immediacy of diagnosis and response to the gradual increase in allogeneic material under the control of a closed-loop feedback control system able to deliver medication as needed, the interlocked continuous processes of transplantation and allogeneic exchange are not over or undershot. That is, the system detects the incipient signs associated with rejection and is therefore able to ‘feel’ its way up to the rate of delivery to both recipient and donor beyond which the allogeneic material of the other would no longer be tolerated. Gradual advancement of the diversion chutes requires vascular servovalves or closed-loop stepper motor-driven valves positioned no differently than would bistable valves to perform the equivalent sudden compound vascular bypass, or sudden switched-organ transplantation.

Continuously monitored as the actuators or effectors in a feedback control system, vascular servovalves can continuously or incrementally increase the volume of reciprocal cross-circulation from 0 to 100 percent during continuous reciprocal cross-circulation, thus minimizing the abruptness of confrontation. In practice, a slight opening or diversion of the valves over time will bring the blend to 50/50, with higher volume exchange rates appropriate where time is a factor, as when recipients are to receive transplants in sequence. Closed loop stepper motor and servomotor-driven valves are reversible and can be moved to any position at any speed. However, stepper motors must reinitialize, or be returned to the reference starting position when turned on, which, depending upon the complexity of the application, can be a serious drawback.

The substitution of less costly but still stiffly responsive closed-loop stepper motors for damped and tuned servomotors responsive to proportional-integral-derivative controllers allows the procedure to be applied in less developed regions. Closed-loop controlled valves allow blood exchange to be adjusted from zero to full and the reverse instantly and at any rate, whether suddenly or in precise increments. That is, unlike solenoid-driven valves which suddenly move the diversion chutes from fully retracted to fully extended, servovalves and closed-loop steppers are continuously variable in speed and direction and as indicated, respond to the commands of the implanted microprocessor fed information from implanted pathophysiological sensors continuously and instantly to drive the procedure.

In addition to the exchange of blood through the valve mainlines, within the intracorporeal space available for the number and capacity of reservoirs which can be placed, the accessory channels can be used to automatically release drugs, leukocytes, erythrocytes, platelets, immunosuppressants, and stem cells into the blood circulated through either or both subjects. Drugs which will not be needed postprocedurally can be suppled through the body surface port and into accessory channels from extracorporeal reservoirs. Since over time, even a slight exchange of blood between recipient and donor will eventually yield a 50/50 mix, to increase flow over the fully open or full diversion setting, blood can also be exchanged through connections between additional corresponding vessels.

Valve-jackets placed for transplantation are those primary for nondrug or endogenous tolerance induction by means of reciprocal cross-circulation as well. Additional reciprocal cross-circulation jackets placed to treat one or more comorbidities can be used to increase the rate of reciprocal cross-circulation as well. While overall inflow and outflow between donor and any one recipient must he equal, the rate of diversion chute extension at a given pair of jackets can allow inflow and outflow complementary to but independent of that of any others, as well as the targeting of medication through the accessory channels, and the collateral actuation of associated devices, such as electrostimulatory, otherwise neuromodulatory, or sphincteric.

Conventional side-entry jackets cannot be used for this purpose, since these are unable to adjust the volume of flow along with the primary vascular servovalves. Additional valves can be used just to increase the volume of reciprocal cross flow between donor and recipient even when only one organ is to be transplanted. However, these are most efficiently employed when the recipient will require the treatment of one or more comorbidities following transplantation.

Increased volume of reciprocal cross-circulation to induce tolerance is obtained by increasing either the number or sum cross-sectional, or flow-through, area of the valves. The volume of blood diverted necessarily equal to that returned, this will ordinarily comprise connections between equivalent of corresponding arterial and venous vessels of donor and recipient through valves with mainlines. However, as indicated, even in a transplantation, reciprocal infusion is not limited to the graft organ. The volume of blood exchanged between paired outlet and inlet valves must be equal; however, the source and destination valve-jackets and vessels need not be the same.

While the volume of blood exchanged between donor and recipient must be equal, where transplantation is not contemplated, valveless jackets can be used to channel blood from one location in the one party to another in the other party. Not ordinarily contemplated, a branched mainline allows blood diverted from a larger vessel to be conveyed into two smaller vessels. Where other organs of the recipient warrant direct medicinal support, these are attended to before connecting unimpaired vessels. Where such connections do not afford the volume of flow required, then large vessels are connected. Once transplantation is completed or after an interval to allow stabilization and recovery, these lines and valves are removed endoscopically.

Once the sensors, valves, lines, and data handling components have been placed, the process of repositioning the graft organ in the circulatory system of the recipient with the automatic release of adjuvant medication such as immunosuppressant, anti-inflammatory, and antimicrobial is carried out automatically according the prescription-program executed by the implanted microprocessor. Excision of the organs, positioning of the graft organ, and closing the recipient are done surgically. That is, setup and closing are surgical, while transplantation and immune tolerance induction are automatic.

Conventional, or valveless, ductus side-entry jackets can be placed to target the delivery of medication through their accessory channels, or sidelines, with or without mainlines. In the event of an unforeseeable complication or error in the prescription-program, the automatic process can be overridden at any time; the automated process does not restrict the collateral manual injection or infusion of drugs or blood products, whether from the donor to the recipient or the reverse, or from third-parties having been banked. Following the transplantation procedure, the feedback control system remains with the recipient to automatically administer the targeted release of drugs.

The inability of blood draws, analysis, and clinical response to attain continuity and immediacy of response is not limited to the organ to be transplanted: end-stage disease of any organ will have resulted in secondary degradation of all the other organs and tissues of the recipient. If not necessitating the transplantation of organs secondarily affected, the placement of valveless ductus side-entry jackets can serve to directly pipeline target medication to these other organs and tissues before, during, and after transplantation of the graft organ. Other organs to be transplanted should be replaced in the same procedure using the same method. Seen from this more encompassing perspective, the ability of manual means to approach the instantaneous response to sensor feedback of an automatic response system is plainly uncompetitive.

Arousal of an adverse alloreaction is recognized and remedial measures applied immediately. The results of a correction are evaluated, and if necessary, an alternative medicine resorted to, a change in electrostimulation applied, or adjustment in an electromechanical actuator such as an artificial sphincter made, and the process continued. The overall response comprises both the medical response and control over the diversion chutes as to whether, which way, when, how far, and how fast to move. When donor and recipient are connected and reciprocal cross-circulation initiated, the master node microprocessors of donor and recipient become penultimate nodes reporting to an extracorporeal overall master control processor, or transplantation master node which oversees and administers the process across both.

Constant maintenance of the recipient in an optimal condition across multiple impairments substantially improves overall health and therefore the odds for a successful outcome. Exceptionally, if either party is markedly hypertensive and the other markedly hypotensive, the difference is controlled by means of drugs or a slight increase in the outflow volume of the hypertensive to the hypotensive. Depending upon dosing times, volumes, and whether the drugs are best kept isolated, these can be delivered through the accessory channels of valveless side-entry jackets. Diseased supply and drainage vessels of organs to be transplanted are transplanted along with the organs. The valves are positioned farther upstream along the arteries and downstream along the veins, the diseased vessels then replaced with their respective organs.

The overall volume of flow between recipient and donor is equal with medically insignificant local deviations in pressure and flow rate due to localized plaque, an aneurysm, or congenital malformation, for example. In both recipient and donor, each organ designated for transplantation represents a channel or arm of control in an implanted adaptive hierarchical control system comprising one or more sensors, effectors, and a dedicated node microcontroller. The local microcontroller will usually be subordinate to higher level node coordinative microcontrollers in the control hierarchy which feed data up to the master node microprocessor responsible for executing the prescription-program.

Sudden compound vascular bypass, or sudden switched-organ transplantation does not preclude, and where the donor-recipient match is less than ideal, is dependent upon, conventional drug and third party or donor-recipient blood and blood products in preparation for, during, and as follow-up to transplantation using reciprocal cross-circulation. Because bistable intravascular diversion valves suddenly move from fully retracted, or closed, with no blood exchanged, to fully extended, or open, so that the blood of the graft organ is suddenly replaced, the immune confrontation, at least without previous tolerogenic induction accomplished manually, is abrupt.

Control over the rate of blood exchange through the reciprocal cross-circulation circuit is precluded, the graft organ abruptly repositioned from the circulatory system of the donor into that of the recipient. With the blood of the recipient passed through the donor organ suddenly with no opportunity for assimilation, tolerance induction using manual means must either precede or succeed transplantation. To accomplish this midprocedurally by calculated and guarded steps which simultaneously transfer the organ between circulatory systems and condition the organ and recipient to one another requires an interval intermediate between full valve closure and open. Moving from fully closed to fully open at a rate controlled by the prescription-program as executed by the master node, vascular servovalves impart this capability.

Because it cannot integrate continuous immunological surveillance and response into the transplantation, sudden compound vascular bypass, or sudden switched-organ transplantation with the supplementation of blood products and drugs before or after a sudden switch transplant cannot duplicate the measured approach of automatic feedback control in guiding the rate at which the incompatibles are blended, adjusting or pausing the process, and if necessary, also releasing moderating or reversal agents to minimize alloreactions. Instantaneous response, minimizing the risk of provoking, and the ability to immediately reverse or lessen the effect of an adverse alloreaction an adverse reaction should it occur necessitate a continuity of diagnosis and response which manual means cannot provide.

Without frequent interruption for blood draws, laboratory testing, and providing instantaneous adjustment in valve settings and/or the release of drugs in response, the process of transplantation proceeds as efficiently as the avoidance of adverse reactions will allow. The prosthetic disorder response system can be loosely thought of as a collection of prosthetic sensors and effectors under the control of a prescription-program executed by an implanted microprocessor acting as a prosthetic or backup automatic response nerve center. Metered compound vascular bypass, or sudden switched-organ transplantation uses the same connections as does sudden compound vascular bypass, or sudden switched-organ transplantation, except that miniature leadscrew or linear motor vascular servovalves rather than bistable plunger solenoid operated valves are used.

Whether the linear actuator is leadscrew or linear motor-based, vascular servovalves are no less capable of supporting a sudden compound vascular bypass, or sudden switched-organ transplantation than are bistable valves; however, even closed-loop stepper motors are more costly. Valves used in a compound vascular bypass, or sudden switched-organ transplant are usually left in place with the cut ends of the vessels anastomosed as shown in FIG. 22B to retain the accessory channel or channels, which are then available to wet with medication and provide a bypass about the anastomoses for treatment or to remove flow when to do so will expedite healing.

Where the risk of immunological provocation is difficult to assess, vascular servovalves can execute a complete transplantation, provide diagnostics, and if the suddenness of the action proves immunologically provocative, the valves are automatically retracted partially, reducing reciprocal cross-circulation flow, or returned to the fully retracted position closing off the circulatory system of the recipient from that of the donor, with resumption of metered compound vascular bypass, or sudden switched-organ transplantation once the reaction is detected to have been reversed. This action proceeds automatically in accordance with the prescription-program as executed by the master node microprocessor.

Sudden switch bistable valves can be returned to the starting position as well, but intermediate settings are not possible, and proceeding blindly without continuous laboratory feedback, the process must be frequently interrupted to allow the taking of blood draws, then waiting for the results from the laboratory, applying the reversal or moderating agents manually, and once again waiting for the laboratory results before the process can be resumed. In contrast, at any moment, upon detection of an adverse reaction, the automatic system can immediately retract the chutes respective of one or more organs for transplant in donor and recipient fully or partially, thus cutting off or reducing the volume of further reciprocal cross-circulation by isolating the circulatory system of each.

Such reversal can be supported by the direct targeting of immunosuppressant or anti-inflammatory medication, for example, through the valve accessory channels, and optionally, through the mainlines. The elimination of interference with implanted sensor function by certain drugs is currently making rapid progress. To implanted must be added worn, such as wrist-worn, sensors (see, for example, Gielen, W. and Gielen, M. 2019. “Two Causes of Palpitations, Detected by Photoplethysmography on a Mobile Phone,” Netherlands Heart Journal 27(1): 52-53; Gielen, W. and Gielen, M. 2019. “Two Causes of Palpitations, Detected by Photoplethysmography on a Mobile Phone,” Netherlands Heart Journal 27(1): 56-57; Kamišalić, A., Fister, I. Jr., Turkanović, M., and Karakatič, S. 2018. “Sensors and Functionalities of Non-invasive Wrist-wearable Devices: A Review,” Sensors (Basel, Switzerland) 18(6). pii: E1714; Georgiou, K., Larentzakis, A. V., Khamis, N. N., Alsuhaibani, G. I., Alaska, Y. A., and Giallafos, E. J. 2018. “Can Wearable Devices Accurately Measure Heart Rate Variability? A Systematic Review,” Folia Medica (University of Plovdiv, Bulgaria) 60(1): 7-20; Al-Eidan, R. M., Al-Khalifa, H., and Al-Salman, A. M. 2018. “A Review of Wrist-worn Wearable: Sensors, Models, and Challenges,” Article 5853917, available Online at https://doi.org/10.1155/2018/5853917; Qureshi, F and Krishnan, S. 2018. “Wearable Hardware Design for the Internet of Medical Things (IoMT),” Sensors (Basel, Switzerland) 18(11). pii: E3812; Talboom, J. S. and Huentelman, M. J. 2018. “Big Data Collision: The Internet of Things, Wearable Devices and Genomics in the Study of Neurological Traits and Disease,” Human Molecular Genetics 27 (R1): R35-R39; Dias, D. and Paulo Silva Cunha, J. 2018. “Wearable Health Devices-Vital Sign Monitoring, Systems and Technologies,” Sensors (Basel, Switerland) 18(8). pii: E2414; Cheung, C. C., Krahn, A. D., and Andrade, J, G. 2018. “The Emerging Role of Wearable Technologies in Detection of Arrhythmia,” Canadian Journal of Cardiology 34(8): 1083-1087; Haghi, M., Thurow, K., and Stoll, R. 2017. “Wearable Devices in Medical Internet of Things: Scientific Research and Commercially Available Devices,” Healthcare Informatics Research 23(1): 4-15; Majumder, S., Mondal, T., and Deen, M. J. 2017. “Wearable Sensors for Remote Health Monitoring,” Sensors (Basel, Switzerland) 17(1). pi: E130).

So long as the diversion chutes in donor and recipient are not fully retracted or extended, the graft organ is situated in the circulatory systems of both donor and recipient, the degree of either dependent upon the degree to which the diversion chutes are extended, that is, to which the valves are open. The reapportionment of circulation on a controlled basis then both physically repositions the organ from the donor to the recipient, effectively transplanting it, and inducing tolerance, possibly to include a measure of cross-chimerization, in one and the same operation. A surface port and jackets with accessory channels placed preprocedurally remain to support the graft organ and the overall health of the recipient.

### Continue Cpr

Life support for the purpose of sustaining a brain dead donor is by means of extracorporeal membrane oxygenation (ECMO) initiated prior to death and maintained until the organs have been harvested. ECMO can preserve the donor over a longer period than can cardiopulmonary bypass, which is more destructive to blood cells. With the gradual, rate controlled, or metered, diversion made possible with vascular servovalves, the blood of donor and recipient mix at a rate determined by the physician in charge and entered into the prescription-program to most quickly and effectively accomplish donor-recipient organ-specific preliminary induction tolerance and possibly cross-microchimerization, hence, provide these with more effective penetrance than would the pre- and midprocedural reciprocal cross transfusion of exogenous or even endogenous blood and blood products alone.

Not drawing primarily upon third party blood products from a blood bank, in extreme circumstances, some mismatching of blood types and not just the size or difference in ventricular absolute stroke volumes between donor and recipient using the metered switch technique may fall within a zone of tolerance (see, for example, Akria, L., Chezar, J., Zisman-Rozen, S., Scheinman, E. J., Zonis, Z., and 9 others 2017. “A Puzzling “Switch” in Blood Type Following Blood Transfusion,” Annals of Laboratory Medicine 37(3): 293-295; Hourani, L., Weingart. C., and Kohn, B. 2017 “Alloimmunisation in Transfused Patients: Serial Cross-matching in a Population of Hospitalised Cats,” Journal of Feline Medicine and Surgery 19(12): 1231-1237; Niggemeier, A., Haberstroh, H. F., Nelson, V. E., and Giger, U. 2000. “An Accidental Transfusion of a Type A Kitten with Type B Blood Causes a Transient Switch from Blood Type A to B,” Journal of Veterinary Internal Medicine 14(2): 214-216; 45) or prove ameliorable through conventional plasma exchange and/or drug-induced desensitization (see, for example, Makroo, R. N., Nayak, S., Chowdhry, M., Jasuja, S., Sagar, G., Rosamma, N, L., and Thakur, U. K. 2017. “ABO Incompatible Renal Transplant: Transfusion Medicine Perspective,” Asian Journal of Transfusion Science 11(1): 45-49; Stojanovic, J., Adamusiak, A., Kessaris, N., Chandak, P., Ahmed, Z., and 5 others 2017. “Immune Desensitization Allows Pediatric Blood Group Incompatible Kidney Transplantation,” Transplantation 101(6): 1242-1246; Urschel. S. and West. L. J. 2016, Op cit. under (b). Metered compound vascular bypass, or sudden switched-organ transplantation above; Song, G. W., Lee, S. G., Hwang. S., Ahn, C. S., Kim, K. H., and 8 others 2014. “A Desensitizing Protocol without Local Graft Infusion Therapy and Splenectomy is a Safe and Effective Method in ABO-incompatible Adult LDLT [living donor liver transplant],” Transplantation 97 Supplement 8: S59-S66, Section 5; Zachary, A, A. and Leffell. M. S. 2014. “Desensitization for Solid Organ and Hematopoietic Stem Cell Transplantation,” Immunological Reviews 258(1): 183-207; Sarkar, R. S., Philip, J., and Yadav, P. 2013. “Transfusion Medicine and Solid Organ Transplant-Update and Review of Some Current Issues,” Medical Journal, Armed Forces India 69(2): 162-167; Magee, C., Clarkson, M., and Rennke, H. 2008. “A Case of Desensitization, Transplantation, and Allograft Dysfunction,” Clinical Journal of the American Society of Nephrology 3(5): 1573-1581; Jordan, S. C., Vo. A., Tyan, D., and Toyota, M. 2006. “Desensitization Therapy with Intravenous Gammaglobulin (IVIG): Applications in Solid Organ Transplantation,” Transactions of the American Clinical and Climatological Association 117:199-211).

The primary object being to maintain perfusion through the graft organ continuously while gradually transition flow thorough the organ from the blood and circulatory system of the donor to that of the recipient, the opposite effect of provoking an adverse alloreaction in either or both recipient and donor may require pre- and/or mid-procedural plasma exchange and platelet and leukoreduction (Sharma, R, R. and Marwaha, N. 2010. “Leukoreduced Blood Components: Advantages and Strategies for Its Implementation in Developing Countries,” Asian Journal of Transfusion Science 4(1): 3-8), for example. Patients on extracorporeal life support are subject to inconsistency in level of immunocompetence (Beshish, A. G., Bradley, J. D., McDonough, K, L., Halligan, N. L. N., McHugh, W. M., and 4 others 2019 Op cit. under 1. Background of the Invention. a. General).

In determining the need for antimicrobials and possibly leukocytes to be administered before and during transplantation, immune function in a donor on extracorporeal life support while still alive, following expiration, and the superimposed effect thereupon of antimicrobials and the transfusion of blood products must be taken into consideration. Where benefit will accrue by intentionally suspending the process to allow time for assimilation in the allogeneic matter and quiescent, the valves are fully retracted, leaving donor and recipient with the mix up to that point, the process then resumed with the valves moved from fully retracted to the positions that had been reached prior to suspension.

Given that rather than to risk complete organ failure, patients nearing irreversible end-stage organ disease should report for a transplant earlier, and that with prompt and diligent mechanical and pharmaceutical support, a ‘brain-dead’ donor can be well preserved for weeks, time will allow immunological preconditioning or acclimatization of donor and recipient to one another, improving the odds for compatibility and a successful outcome. Organs to be harvested for transplantation should not only be kept continuously perfused within the life supported donor but provided with any ancillary medication, nutritional substances, and support such as inotropes, vasodilators, antimicrobials, and electrostimulatory neuromodulation for example, as necessary to best preserve their organs for transplantation (Todd, P. M., Jerome, R. N., and Jarquin-Valdivia, A, A. 2007, Op cit. under section 1a. Background of the Invention. General).

By increasing the diversion of blood flow between or among the recipient or recipients and donor gradually rather than through an abrupt repositioning of the graft organ into the circulatory system of the recipient or recipients as in sudden compound vascular bypass, or sudden switched-organ transplantation, metered compound vascular bypass, or sudden switched-organ transplantation allows the blood of each to slowly increase in relative proportion through the spleen, thymus, lymph nodes, marrow and all other organs and tissues of each.

The time reciprocal cross-circulation continues and the extension of the diversion chutes determines the rate at which the blood of the donor and one or more recipients blend, so that continued long enough, even slight diversion will result in a 50/50 blend with participants equal in blood volume. Full extension of the chutes will cause the blood of donor and recipient to quickly blend and is most likely to provoke an adverse allogeneic reaction. To avoid hyper- or hypovolemia of any, the rate of diversion from donor to one or more recipients and the reverse must be equal.

The rate of chute advancement is dynamically controlled by a microcontroller driven by the data received from sensors to include immunosensors sending data on the blood pressure and state of antibody production or mobilization in either body. The program calculated to proceed in small increments until an immune response is detected or rises to a certain intensity, this should reduce the odds for immunological recoiling and rejection and potentially reduce the need for mid- or postprocedural medication.

Were automated monitoring and response unavailable, exposure to the blood of the donor would have to be interrupted by taking blood draws, sending these to the laboratory and waiting for the results, infusing reversal agents and accomplishing transfusion from the recipient to the donor and the reverse as necessary, making intermittent manual means lacking in the continuity needed to identify the limit of admixture, that is, the exact level at which the blood of donor and recipient might continue to be mixed while avoiding an immune response.

The rate at which the diversion chutes advance is controlled by a microprocessor implanted in the recipient that coordinates numerous immunological and physiological indicia sent to it by sensor data-supplied microcontrollers positioned in both donor and recipient. These local microcontrollers are subordinate in the control hierarchy in accordance with the prescription-program keyed to act on the diagnostics to maintain optimal recipient homeostasis throughout the procedure as well as control the rate and extent of chute advancement. As indicated, when the donor and one or more recipients are connected, an extracorporeal master processor oversees the process, which must consider the continued use of the deceased donor.

The administration of gene therapy or cross-chimerizing and/or mutually exogenous desensitizing agents through the exchange of marrow, stem cells, or eventually gene therapy with “naked,” or plasmid (see, for example, Sabbieti M G I, Lacava, G., Amaroli, A., Marchetti, L., Censi, R., Di Martino, P., and Agas, D. 2018. “Molecular Adjuvants Based on Plasmids Encoding Protein Aggregation Domains Affect Bone Marrow Niche Homeostasis,” Current Gene Therapy 17(5): 391-397; Wolff, J, A. and Budker, V. 2005. “The Mechanism of Naked DNA Uptake and Expression,” Advances in Genetics 54:3-20; Liu, F. and Tyagi, P. 2005. “Naked DNA for Liver Gene Transfer,” Advances in Genetics 54:43-64).

DNA, for example, is administed with a fine needle or hypotube tip-equipped fiber endoscope injection through an opening in a small port at the body surface which leads to the accessory channel of the valve or valveless jacket at the treatment site or sites wanted. Substances to be kept separate are passed released from implanted reservoirs automatically or injected through different accessory channels of the jacket at the target site. Transfection is by direct injection into the portal vein. Drugs to the donor and the recipient and recipients each flow to the other or others in proportion to the continuously varied reapportionment in blood.

Any drug or other substance which should be kept from either or both participants should be neutralized or removed prior to the initiation of reciprocal cross-circulation. The method depends upon the substance. Various methods for eliminating or reducing the objectionable substance to an innocuous level include the use of drugs, plasmapheresis, and bonding to a superparamagnetic carrier micro- or nanoparticle for magnetic detention or uptake. Within the limited difference in size between donor and recipient, the excess volume and ventricular absolute stroke volume of that larger can either be minimized in the mix by fully retracting the chutes to close off reciprocal cross-circulation, or the diversion chutes can be intentionally kept from complete extension, so that the excess is shared by the recipient; that is, the mix between them is then disproportionately that of the larger party.

Contrariwise, the overall interval for chute movement can intentionally be made less than that necessary for full blending-probably will be due to an adverse immune response before this level is reached—and the controlling microprocessor can be programmed to suddenly advance the chutes from any position to full retraction, thereby isolating the circulatory systems. Generally, chute advancement is completely flexible in rate and direction of movement, and can be made complete at any moment of advanced at a slower rate when either donor or recipient is larger, as when one is a male and the other a female.

If a preclusive reaction has not been provoked, continuation of flow with the chutes fully extended beyond a 50/50 mixture allows a smaller subject to share the additional blood of the larger. The target or end-point for tolerance induction of the recipient can be set as a level of resistance to further induction or with remedial measures, a point beyond this level. When either is reached, the diversion chutes are suddenly advanced to the fully retracted position, isolating the circulation of the recipient from that of the donor. Otherwise, sudden full extension leaves the donor with the proportional mix attained at the time it is accomplished. Thus, any blend can be obtained up to 50/50, and with a larger donor, 50 plus/50.

The system monitors the overall status of the donor and transplant organ prior to expiration, and responds to the pathophysiological changes in these which ensue upon brain death and as donor-recipient reciprocal cross-circulation and transplantation proceed (see, for example, Carpenter, D. J., Granot, T., Matsuoka, N., Senda, T., Kumar, B. V., and 10 others 2018. “Human Immunology Studies Using Organ Donors: Impact of Clinical Variations on Immune Parameters in Tissues and Circulation,” American Journal of Transplantation 18(1): 74-88; Belhaj, A., Dewachter, L., Rorive, S., Remmelink, M., Weynand, B., and 7 others 2017. “Mechanical Versus Humoral Determinants of Brain Death-induced Lung Injury,” Online, PLoS [Public Library of Science] 12(7): e0181899; Yip, H. K.5, Lee, M. S., Sun, C. K., Chen, K. H., Chai, H. T., and 7 others 2017. “Therapeutic Effects of Adipose-derived Mesenchymal Stem Cells against Brain Death-induced Remote Organ Damage and Post-heart Transplant Acute Rejection,” Oncotarget 8(65): 108692-108711; Li, Y., Xiong, Y., Zhang, H., Li, J., Wang, D., and 8 others 2017. “Ginkgo Biloba Extract EGb761 Attenuates Brain Death-induced Renal Injury by Inhibiting Pro-inflammatory Cytokines and the SAPK and JAK-STAT Signalings,” (Nature Research) Scientific Reports 7:45192; Stone, J. P., Mohamud, M., Amin, K., Critchley, W. R., Edge, R. J., Clancy, M. J, Ball, A, L., and Fildes, J, E. 2017. “Characterizing the Early Inflammatory Contribution of the Donor Kidney following Reperfusion,” Nephrology, Dialysis, Transplantation 32(9): 1487-1492; Sung, P. H., Lee, F. Y., Lin, L. C., Chen, K. H., Lin, H. S., and 8 others 2017. “Melatonin Attenuated Brain Death Tissue Extract-induced Cardiac Damage by Suppressing DAMP [damage-associated molecular pattern] Signaling,” Oncotarget 9(3): 3531-3548; Stone, J. P., Ball, A, L., Critchley, W. R., Major, T., Edge, R. J., Amin, K., Clancy, M. J., and Fildes, J, E. 2016. “Ex Vivo Normothermic Perfusion Induces Donor-derived Leukocyte Mobilization and Removal Prior to Renal Transplantation,” Kidney International Reports 1(4): 230-239; Fung, A., Zhao, H., Yang, B., Lian, Q., and Ma, D. 2016. “Ischaemic and Inflammatory Injury in Renal Graft from Brain Death Donation: An Update Review,” Journal of Anesthesia 30(2): 307-316; Ploeg, R. J. and Friend, P. J. 2015. “New Strategies in Organ Preservation: Current and Future Role of Machine Perfusion in Organ Transplantation,” Transplant International 2015 28(6): 633; Ponticelli, C. 2014. “Ischaemia-reperfusion Injury: A Major Protagonist in Kidney Transplantation,” Nephrology, Dialysis, Transplantation 29(6): 1134-1140; Watts, R. P., Thom, O., and Fraser, J, F. 2013. “Inflammatory Signalling Associated with Brain dead Organ Donation: from Brain Injury to Brain Stem Death and Posttransplant Ischaemia Reperfusion Injury,” Journal of Transplantation 2013:521369; van der Hoeven, J. A., Molema, G., Ter Horst, G., Freund, R, L., Wiersema, J., van Schilfgaarde, R., Leuvenink, H. G., and Ploeg, R. J. 2003. “Relationship Between Duration of Brain Death and Hemodynamic (In) stability on Progressive Dysfunction and Increased Immunologic Activation of Donor Kidneys,” Kidney International 64(5): 1874-1882; van Der Hoeven, J. A., Ter Horst, G. J., Molema, G., de Vos, P., Girbes, A. R., and 5 others 2000. “Effects of Brain Death and Hemodynamic Status on Function and Immunologic Activation of the Potential Donor Liver in the Rat,” Annals of Surgery (Philadelphia, Pennsylvania) 232(6): 804-813).

Under sudden and metered compound vascular bypass, or sudden switched-organ transplantation where perfusion is continuous and life support is not shut down until after transplantation has been completed with sufficient tolerance having been induced, the risk of degradation from these sources and infection should be much less than when life support is shut down. Given the impact of death on the donor, the administration of anti-inflammatoires, immunosuppressants, and the continuation of circulation through the terminal event should avert much of the risk of immune rejection mediators aroused by death even before the further provocation of an adverse immune reaction following transplantation.

For the recipient, the system monitors overall status prior to, during, and after reciprocal cross-circulation and transplantation, and thereafter, overall status and the condition of the recipient and graft organ. For the recipient, the implant system for monitoring and automatically supporting the transplant remain in use to the end of life. Ideally, in the effort to have kept the prospective donor alive, homeostasis had been optimized by such means through the implantation of an automatic adaptive disorder response system while the donor remained alive.

The need to implant an automatic adaptive prosthetic disorder response system in both recipient and donor is explained by the number of diagnostic variables to be monitored and the number of therapeutic variables to be adjusted in a coordinated manner in order to achieve optimal homeostasis throughout this form of merged reciprocal cross-circulation and transplantation. An implant system is implemented in recipient and donor before the two are connected, each system optimized to deal with the condition of its respective host. Either system operates under the overall control of a master node microprocessor executing a prescription-program devised to optimize the homeostatic condition of its host across all variables.

Such a control system is hierarchical, in that physiological and chemical parameter sensors feed data to subordinate level or first-line node microcontrollers which supply their data to superior nodes in the hierarchy to coordinate the data at the local level, the integrated data from the regional nodes passed up the control hierarchy through intervening nodes or directly to the master node. Once the donor and recipient are connected, the implanted master controller of each is placed in subordinate hierarchical relation to an extracorporeal processor that becomes the master node.

In the effort to preserve the deceased in a donor-ready state, the need of the prospective recipient or recipients must override any competing consideration: as to the order of organ harvesting in relation to continued preservation of other organs during aggressive donor support (see, for example, Pandit, R. A., Zirpe, K. G., Gurav, S. K., Kulkami, A. P., Karnath, S., and 10 others 2017. “Management of Potential Organ Donor: Indian Society of Critical Care Medicine: Position Statement,” Indian Journal of Critical Care Medicine 21(5): 303-316; Shetty, V, L., Mali, S. S., Shetty, S. V., and Shinde, P, D. 2017, Op cit.; Kumar, L. 2016, Op cit.; Dhanani, S. and Shemie, S, D. 2014. “Advancing the Science of Organ Donor Management,” Critical Care 18(6): 612; Todd, P. M., Jerome, R. N., and Jarquin-Valdivia, A, A. 2007, Op cit.), cardiopulmonary function is effectively simulated with the aid of an extracorporeal membrane oxygenator and if appropriate, a cardioverter-defibrillator.

Kidney function can be substantially sustained through hemodialysis and adjustment in the blood levels of minerals normally processed by the kidneys. It is the liver, directly or indirectly involved in every pathway of which the function is not simulable as is that of a pump or filter, that once removed most severely impacts the usability of the remaining organs. For this reason, the heart, lung or lungs, and kidneys are best removed earlier, with the liver harvested last, or if exigency makes it necessary, performed simultaneously with the harvesting of the other organ or organs. Liver dialysis?

A benefit of sudden and metered compound vascular bypass, or sudden switched-organ transplantation is minimization in procedural duration, dissection, and the avoidance of general anesthesia, the more beneficial when multiple organs are transplanted, such as the heart, kidneys, and pancreas to treat end-stage cardiorenal syndrome, for example, where the graft organ such as the heart donor is dead and a kidney is taken from a living donor or both organs are taken from one or two different donors. Other combination organ syndromes where another organ is secondarily impaired due to impairment in that primary are numerous. In such multiple organ transplantation, the volume of blood incurrent into the recipient must exactly match that excurrent to the donor or donors, which volume is dictated by the volume flow through of the organ or organs transplanted.

Even sustained on extracorporeal membrane oxygenation with aggressive maintenance, the organs and tissues of a deceased subject inevitably deteriorate, encouraging the identification of matched donors and recipients as early as possible. The establishment of deceased donor sustainment centers as addressed above in section 2a, entitled Background of the Invention. General, in the subsection thereof of like title will facilitate preliminary matching. As the end stage in a prospective deceased donor for organ utilization is approached, the transition to improved tolerance attainable through the microchimerism imparted by reciprocal cross-circulation alone will require to be supplemented with proportionally higher doses of immune tolerance inducing medication. This does not, however, warrant dispensing with metered rather than use of sudden compound vascular bypass, or sudden switched-organ transplantation.

Donors to provide more than one organ to the same recipient can be prepared at the outset with jackets positioned accordingly; the extent to which the valves are opened varying inversely as their number. With two simultaneous recipients, the valves in the donor are set to the sum of the recipient valve openings. Only after the vascular servovalves for transplantation have been placed and their sufficiency to support tolerance induction having been confirmed, are any supplementary components such as conventional side-entry jackets, sensors, and electrostimulatory devices placed. This assures that sudden compound vascular bypass, or sudden switched-organ transplantation, of which only intravascular, or switched-organ valves, are capable, will be possible should the need therefor arise before all the components can be placed.

When urgency is not pressing, having preplaced vascular servovalves rather than plunger solenoid or sudden switch valves will additionally allow transplantation preliminary tolerance induction with automatic surveillance and control. In exigent circumstances, more than one recipient can be connected to provide blood to and through different organs of the donor to accomplish tolerance induction and cross-microchimerization as well as switch the graft organ from the donor to the recipients in a more than two-way reciprocal cross-circulation arrangement.

Otherwise, it is of benefit to schedule transplantations from the same donor in relatively close succession. When the heart is urgently needed and taken first, sustainment of the other organs and tissues is by continuation on life support with adjustment of the extracorporeal membrane oxygenator settings if necessary to sustain arterial and venous flow and oxygenation of the blood in the absence of a heart. With the lungs remaining in the donor, the need for a ventilator, breathing tube, and adjunct medical support will usually persist.

All diversion valves remaining connected to the stumps of the graft organ when excised and placed in the recipient, to minimize donor blood loss following excision of the graft organ and continue perfusion without interruption of remaining organs needed for transplantation, supply and drainage vessels of the excised organ are anastomosed with preservation to allow use of the other organs sustained by life support or an artificial heart with endocrine, temperature, and medicinal support as necessary. Alternatively, the jackets used have raised facing end rims which can be clamped together to anastomose the stumps ends as described above in section 2. Background of the Invention. a. General.

Less conventionally, the deceased donor can be sudden switched from his native to an orthotopically positioned artificial heart. Such represents a use for these devices other than to bridge the period pending the availability of a replacement heart. Transfer from the native heart and/or lungs to a membrane oxygenator or implantable artificial heart is without the need for switched-organ diversion of the blood supply and drainage to the heart and lungs. However, reconnection of the native vessels to the replacement graft organ, which otherwise poses reperfusion injury as a serious consequence does. The bloodstreams of the participants gradually blended, the rate of deterioration in the different organs intended for the recipients must be taken into consideration. Where time is limited, the donor may be connected to multiple recipients.

With the hearts of all the participants beating, utilization of several solid organs of the brain-dead donor can usually be accomplished without the need for supplementary pumping. A pump may be required when, for example, a recipient with end-stage heart failure lacks the blood pressure and ventricular absolute stroke volume necessary to contribute flow through the arrangement and the administration of an inotrope, cardiac myosin activator or other drug causes a sudden arrest before the heart and/or other organs could be replaced (see, for example, Kaplinsky, E. and Mallarkey, G. 2018. “Cardiac Myosin Activators for Heart Failure Therapy: Focus on Omecamtiv Mecarbil,” Drugs in Context 7:212518; Kajimoto, K., Sato, N., Sakata, Y., Takano, T., and 186 collaborators 2013. “Relationship between Systolic Blood Pressure and Preserved or Reduced Ejection Fraction at Admission in Patients Hospitalized for Acute Heart Failure Syndromes,” International Journal of Cardiology 168(5): 4790-4795).

The total volume of blood outflow and inflow for any one participant must be equal, which—absent counteracting measure—would require an outflow from a singular recipient to two donors, double the output. However, different organs in different patients can be independently metered, which can further support the prospective donor organ with medication and nutrients through the accessory channel coursing along the underside of the diversion chute and/or a nonjacketing side-entry connector.

Within this total, flow through any one or more valve-jackets can vary, thus accommodating. Coordination of otherwise autonomous circuits to overall homeostasis a concern, the circuits are positioned as subordinate nodes under the control of a microprocessor as master node, in turn subordinated to the extracorporeal master processor during the transplantation. The graft organ removed, the valve-jackets are left on the stumps to allow the continued direct pipeline targeting of medication to the transplant. The prosthetic disorder response system acts on physiological and pathophysiological data obtained from the implanted sensors.

The raw data is processed first by subordinate node microcontrollers, which pass their outputs up through the control hierarchy to more comprehensive data coordinative nodes, thence, to the master node microprocessor responsible for executing the prescription-program. Following transplantation, the sudden switch or servovalves are left in the recipient to-allow the automatic targeting of medication directly to the treatment site and to reduce the need for frequent follow-up visits to the clinic. The cost of later procedures can be materially reduced by recovering the more expensive components such as the valves and master control microprocessor before the donor and recipient are cremated or buried.

The use of either sudden or metered compound vascular bypass, or sudden switched-organ transplantation outside the context of oversight by an automatic adaptive disorder response system in no way nullifies the use of conventional means of diagnosis and the exchange of blood products and other means for accomplishing tolerance induction and cross-microchimerization, which in fact are facilitated by the availability of accessory channels which directly pipeline target the materials used to the treatment sites. When the mainlines are several feet in length, the drop in blood pressure when the valves open is prevented by filling the mainlines with saline solution and/or blood before initiating reciprocal cross-circulation.

Simultaneous opening of valves to divert all flow between adjacent vessels is suitable in an extracardiac complete or dextro-transposition of the great arteries where all or nearly all flow is diverted from the one vessel to the other. For this, the two valves are accessible for synchronous switching, allowing the use of manually (FIG. 3a) or solenoid-controlled valves (FIG. 3b). Sudden compound vascular bypass, or sudden switched-organ transplantation where all flow is diverted from the native to the donor organ requires a number of less accessible valves that must be switched simultaneously, necessitating the use of solenoid valves controlled from a common switch.

Metered compound vascular bypass, or sudden switched-organ transplantation, where the transfer of the graft organ from the circulatory system of the donor into that of the recipient, is gradual, responsive to sensors that detect the mediators of tolerance induction, and the release of remedial medication if necessary is automatic. Blood is diverted between the parties on a measured basis for transit of compensatory blood in equal volume from another vessel, usually the vessel in the donor corresponding to the source vessel in the recipient.

The object is to connect a prospective deceased adult, prenate, neonate, or child donor to a similar recipient to join their circulatory systems upon loss of brainstem function so that the two can be reciprocally cross-circulated continuously. While generally less problematic prior to maturity of the immune system, cross tolerance is best initiated before the donor under aggressive donor management degrades to any extent. Ideally, rather than to transport excised organs, even under normothermic pulsatile perfusion, prospective organ recipients are transported to the donor, not the reverse.

This also comports with the use of one donor to supply transplant organs, whether successively or simultaneously, to more than one recipient. The master node adjusts the servovalve or servovalves, the release, if any, of inotropes and vasoactive substances in donor and recipient to achieve proper blood pressure during and after organ transplantation, extracardiac correction of complete or nearly complete or dextro-transposition of the great arteries (FIG. 4), and transplantation of the heart to a patient with transposition of the great arteries, for example. Control thus is across both recipient and donor in relation to one another and internally with respect of each, before, during, and after reciprocal cross-circulation.

In addition to the automatic release of anti-inflammatoires, diversion valves can alleviate localized, such as pulmonary arterial or venous hypertension by moving blood from the pulmonary to other vessels. Extraordinarily, more complicated chimerization where organs are to be obtained from more than one donor or donated to more than one recipient can prompt a three or four-way connection, for example. In a three-way connection, the donor on life support is positioned at the center with either of two prospective recipients positioned to either side.

Such an arrangement allows the cross-microchimerization of the three so that, for example, the heart and lungs of the deceased donor can be transplanted into one or both of the others, and the others can exchange organs. By contrast, living donors and recipients of a kidney, kidney and pancreas, portion of the liver, lung or portion of a lung are not connected. If remote, the harvested organ is kept continuously perfused with the aid of an organ transport support system.

Since tolerance induction and cross-chimerization best precede transplantation, perfusion of the graft organ is exposed to the blood and blood products of the prospective recipient upon delivery of a neonate donor and diversion valve-jackets placed endoscopically as soon as the condition of both donor and recipient allow. Anencephalic prenates and neonates usually exhibit cardiopulmonary function upon delivery, however, rarely capable of surviving past birth and then at most, for only a few months, they are considered alive, making the harvesting of their organs unlawful.

Presenting the most severe of neural tube defects, these patients ordinarily die on or a day or so after birth. Poorly understood, the condition appears to involve a complex interplay of multiple genetic defects and environmental factors, ischemic injury during prenatal development suspected in this regard. Stories claiming the survival of an anencephalus who developed a brain over a period of years after birth appear to relate to less severe neural tube defects.

Congenital defects often pleiotropic, as is, for example, heart and other solid organ defects in a trisomy 21, the organs from donors with severe genetic defects are suspect for other genetic defects and therefore unsuitable to serve as organ donors (see, for example, Watts, R. P., Thom, O., and Fraser, J, F. 2013. “Inflammatory Signalling Associated with Brain Dead Organ Donation: from Brain Injury to Brain Stem Death and Posttransplant Ischaemia Reperfusion Injury,” Journal of Transplantation 2013:521369; Canadian Paediatric Society 2005. “Use of Anencephalic Newborns as Organ Donors,” Paediatric and Child Health 10(6): 335-337). Organs taken from anoxic brain-injured donors are also injured and often rejected (Kramer, A. H., Baht, R., and Doig, C. J. 2017. “Time Trends in Organ Donation after Neurologic Determination of Death: A Cohort Study,” Canadian Medical Association Journal Open 5(1): E19-E27).

Such literature as exits is largely ill-advised and alludes to procedures that would fail and sources for organs that would likely result in rejection if not hyperacute T cell-mediated allograft rejection. Neonates such as anencephalics usually have not one but plural genetic defects, and still alive or having died, are properly excluded as donors. For example, any component of the urinary tract in neonates with severe neural tube defects may also be defective (see, for example, Diniz, A. L, L., Sampaio, F. J. B., and Favorito, L, A. 2020. “Anencephaly Alters Renal Parenchyma Volume in Human Fetuses?,” International Brazilian Journal of Urology 46(6): 1075-1081)(see also Steinberg, A., Katz, E., and Sprung, C. L. 1993. “Use of Anencephalic Infants as Organ Donors,” Critical Care Medicine 21(11): 1787-1790; Truog, R, D. and Fletcher, J, C. 1990. “Brain Death and the Anencephalic Newborn,” Bioethics 4(3): 199-215; Drugan, A., Evans, W. J., and Evans, M. I. 1989. “Fetal Organ and Xenograft Transplantation,” American Journal of Obstetrics and Gynecology 160(2): 289-293; Shewmon, D. A., Capron, A. M., Peacock, W. J., and Schulman, B. L. 1989. “The Use of Anencephalic Infants as Organ Soures. A Critique,” Journal of the American Medical Association 261(12 (: 1773-1781; Fletcher, J. C., Robertson, J. A., and Harrison, M, R. 1986. “Primates and Anencephalics as Sources for Pediatric Organ Transplants. Medical, Legal, and Ethical Issues,” Fetal Therapy 1(3-3): 150-164; Harrison, M, R. 1986. “Organ Procurement for Children: The Anencephalic Fetus as Donor,” Lancet (London, England) 2(8520): 1383-1386). The preoccupation with ‘ethical’ issues concerning such propositions, unworkable, uniformed, and doomed, has conspicuously disappeared from the literature.

Provided they have been maintained on life support since expiring, deceased patients without disqualifying conditions such as old age or any of a number of genetic defects having been medically managed prior to and following a sudden cardiac arrest are suitable organ donors (Hawkes, M, A. and Rabinstein, A, A. 2019. “Neurological Prognostication after Cardiac Arrest in the Era of Target Temperature Management,” Current Neurology and Neuroscience Reports 19(2): 10; Tsetsou, S., Novy, J., Pfeiffer, C., Oddo, M., Rossetti A, O. 2018. “Multimodal Outcome Prognostication after Cardiac Arrest and Targeted Temperature Management: Analysis at 36° C.,” Neurocritical Care 28(1): 104-109; Tsetsou, S., Novy, J., Pfeiffer, C., Oddo, M., Rossetti A, O. 2018. “Multimodal Outcome Prognostication after Cardiac Arrest and Targeted Temperature Management: Analysis at 36° C.,” Neurocritical Care 28(1): 104-109; Sandroni, C., D'Arrigo, S., and Nolan, J. P. 2018. “Prognostication after Cardiac Arrest,” Critical Care 22(1): 150; Nguyen, P, L., Alreshaid, L., Poblete, R. A., Konye, G., Marehbian, J., and Sung, G. 2018. “Targeted Temperature Management and Multimodality Monitoring of Comatose Patients after Cardiac Arrest,” Online, Frontiers in Neurology 9:768; Sandroni, C., D'Arrigo, S., Callaway, C. W., Cariou, A., Dragancea, I., Taccone, F. S., and Antonelli, M. 2016. “The Rate of Brain Death and Organ Donation in Patients Resuscitated from Cardiac Arrest: A Systematic Review and Meta-analysis,” Intensive Care Medicine 42(11): 1661-1671; West, S., Soar, J., and Callaway, C. W. 2016. “The Viability of Transplanting Organs from Donors who Underwent Cardiopulmonary Resuscitation: A Systematic Review,” Resuscitation 108:27-33; Sandroni, C., Adrie, C., Cavallaro, F., Marano, C., Monchi, M., Sanna, T., and Antonelli, M. 2010. “Are Patients Brain-dead after Successful Resuscitation from Cardiac Arrest Suitable as Organ Donors? A Systematic Review,” Resuscitation 81(12): 1609-1614).

Ischemic injury pertains to any intrauterine or intrapartum death in which anoxia had played a part. Otherwise normal prenates and neonates with congenital heart defects too severe to be repaired to normal circulatory function are, however, ideal candidates for sudden switch heart transplantation, first, because until a heart transplant, the abnormal circulation due to the defective heart would result in secondary maldevelopment of all organs and tissues, and second, because the undeveloped immune system poses less risk of rejection. Where late-term, or post-viability, abortion is legal, the procurement of a replacement heart should pose little difficulty.

Differential organ cross-chimerization is best applied both to potential donors and recipients, reduced reliance upon immunosuppressants lessening the risk of infection for both. Differential organ chimerization with the aid of direct piping from a port at the body surface to each organ should prove more effective for transplantation involving individual donors to multiple recipients, multiple donors to a single recipient, and multiple donors to multiple recipients as well as multiple organ transplantation from one individual to another. More significantly, the emplacement of an automatic prosthetic disorder response system in patients should forestall expiration, so that organs are not called for as soon as these become available, precluding measures that would improve transplantation tolerance.

Rather than suddenly switching the donor organ from the circulatory system of the donor into the circulatory system of the recipient using solenoid-driven side-entry vascular valves, using side-entry vascular servovalves, the rate of diversion chute advancement can be precisely controlled over a gradual period. The process of reciprocal cross-circulation allows the optimization of reciprocal, or donor-recipient mutual, immune tolerance induction through cross-micro-chimerization and if necessary, the addition of immune tolerance-inducing drugs.

The prescription-program is devised to actively seek the optimal condition of homeostasis for the recipient given the conditions of the graft organ and recipient. This gradual rather than sudden action allows the reciprocal endogenous immunotolerance induction of both recipient and donor by reciprocal cross-circulation either initiated before or immediately after death. Otherwise, to the inflammation of the ‘cytokine storm’ initiated by death is appended the added injury of stopping life support and excision.

This gradual introduction of cross allogeneic constituents minimizes stark confrontation by introducing the allogeneic material on a tightly controlled and measured basis without the interruption or reduction in flow between donor and recipient with a concurrent delivery of immunosuppressives (immunosuppressants, anti-rejection drugs) such as mycophenolate mofetil, tacrolimus, azathioprine, and cyclosporin, and anti-inflammatoires that would otherwise allow the ‘cytokine storm’ initiated upon death to proceed unimpeded while blood is drawn, tested, and adjustment made only after the results of testing return from the laboratory. The merger of cross acclimation as the graft organ is transferred from the donor to the recipient is thus significant for graft survival.

As addressed herein, donor chimerization does not pertain to secondary chimerization of the engrafted organ following transplantation (Starzl, T. E., Demetris, A. J., Murase, N., Trucco, M., Thomson, A. W., and Rao, A. S. 1995. “The Changing Immunology of Organ Transplantation,” Hospital Practice 30(10): 31-4, 37-42; Slavin, S. 2002. “Maternal-Fetal Relationship, Natural Chimerism and Bilateral Transplantation Tolerance as the Basis for Non-myeloablative Stem Cell Transplantation,” International Journal of Hematology 76 Supplement 1:172-175), but rather intentional treatment of the donor prior to organ retrieval to better match the recipient. The longevity of deceased, ideally banked, patients for organ recovery can be much increased through the placement before or upon cerebral death of an automatic adaptive hierarchical ambulatory prosthetic disorder response control system as described in U.S. Pat. No. 11,759,186.

Following death, potential organ donors fitted with a system while living should seldom require review of the prescription-program to assure optimal organ preservation. Then, time permitting, once identified, the prescription-program of the donor is reviewed for any adjustment that would better serve the specific recipient, who likewise should be fitted with such a system to best accommodate transplantation; with hematopoietic and endocrine function continued intact under machine support (Miller, F, G. and Sade, R. M. 2014, Op cit.), chimerization of the donor remains possible to complement chimerization and immunosuppression of the recipient (Pandit, R. A., Zirpe, K. G., Gurav, S. K., Kulkarni, A. P., Karnath, S., and 10 others 2017. “Management of Potential Organ Donor: Indian Society of Critical Care Medicine: Position Statement,” Indian Journal of Critical Care Medicine 21(5): 303-316; Girlanda, R. 2016. “Deceased Organ Donation for Transplantation: Challenges and Opportunities,” Online, World Journal of Transplantation (Pleasanton, California) 6(3): 451-459; Nelson, H. M., Glazier, A. K., and Delmonico, F. L. 2016. “Changing Patterns of Organ Donation: Brain Dead Donors are Not Being Lost by Donation after Circulatory Death,” Transplantation 100(2): 446-450; Kotloff, R. M., Blosser, S., Fulda, G., Malinoski, D., Ahya, V. N., and 37 others 2015. “Management of the Potential Organ Donor in the ICU: Society of Critical Care Medicine/American College of Chest Physicians/Association of Organ Procurement Organizations Consensus Statement,” Critical Care Medicine (6): 1291-1325).

Identifiying donors and recipients as early as possible, promptly listing these by the organ sharing organization, and emplacement of an adaptive automatic prosthetic disorder response system in both while the donor remains alive would afford an interval to improve the immunological match between the two. It is possible to place the implanted sensor readings and a wireless body area sensor network (see, for example, Cai, Z., Seyedi, M., Zhang, W., Rivet, F., and Lai, D. T. H. 2017. “Characterization of Impulse Radio Intrabody Communication System for Wireless Body Area Networks,” Journal of Medical and Biological Engineering 37(1): 74-84; Kim, B. S., Kim, K. H., and Kim, K. I. 2017. “A Survey on Mobility Support in Wireless Body Area Networks,” Sensors (Basel) 17(4). pii: E797; Akbar, M. S., Yu, H., and Cang. S. 2017. “IEEE [Institute of Electrical and Electronics Engineers technical standard number] 802.15.4 Frame Aggregation Enhancement to Provide High Performance in Life-critical Patient Monitoring Systems,” Sensors (Basel) 17(2). pii: E241; Li, R., Lai, D. T. H., and Lee, W. 2017. “A Survey on Biofeedback and Actuation in Wireless Body Area Networks (WBAN),” IEEE Reviews in Biomedical Engineering August 10; Zhang, Y., Zhang, B., and Zhang, S. 2017. “A Lifetime Maximization Relay Selection Scheme in Wireless Body Area Networks,” Sensors (Basel) 17(6). pii: E1267; Xu, Z., Hu, T., and Song, Q. 2017. “Bulk Data Dissemination in Low Power Sensor Networks: Present and Future Directions,” Sensors (Basel) 17(1). pii: E156; Nepal, S., Pudasani, A., and Shin, S. 2017. “A Fast Channel Assignment Scheme for Emergency Handling in Wireless Body Area Networks,” Sensors (Basel) 17(3). pii: E477; Ghamari, M., Janko, B., Sherratt, R. S., Harwin, W., Piechockic. R.5., and Soltanpur, C. 2016. “A Survey on Wireless Body Area Networks for eHealthcare Systems in Residential Environments,” Sensors (Basel) 16(6). pii: E831) in either in intermittent communication with the microcontroller of the other, an advanced prescription-program then able to effect adjustments in therapy to fine tune organ matching between the two, with the status of this interaction transmitted to the medical staff.

Wireless body area sensor networks and sensors are addressed in U.S. Pat. No. 11,759,186, entitled Ductus Side-entry Jackets and Prosthetic Disorder Response Systems and U.S. Pat. No. 11,013,858. Compared to chimerization of the prospective recipient alone, previously chimerizing the donor to the recipient as well further reduces the shock to the donor organ of extirpation followed by grafting into a new, immunologically alien milieu, and therewith, the risk of graft versus host disease.

By allowing some protraction in the lives of donors and recipients, these measures facilitate cross-chimerization in preparation for transplantation. Currently chimerization of a donor to a prospective recipient is hindered by the press of time; donors are identified after death, recipients when already dying, so that the need for organs is exigent and unremitting. In this circumstance, effective means for preserving and better preparing deceased, ideally banked, donors is beneficial. When a donor becomes available, there is already a long list of patients with an urgent need for an organ.

This lack of time for medical professionals to respond with greater efficiency accounts for the lack of articles in the literature pertaining to reciprocal cross-chimerization between a specific donor and a specific recipient and the time required for chimerization to take effect. In many, but not all instances, the procurement of a continuously perfused, normothermic transplant organ is not possible; however, the prospective donor can often be recognized before death as harboring a potentially fatal condition, so that the emplacement of such an automatic system might well serve to extend the life of the donor as well as initiate chimerization when the prospective recipient is identified.

The same may be said of the recipient, who likewise will present signs and symptoms indicative of an imminently fatal condition, justifying the emplacement of such a system to treat the existing pathology as well having been prepositioned to initiate chimerization when a match is found. Such an approach both lessens urgency should the recipient suddenly decompensate and improves the prospects for both donor and recipient, who ideally will both recover, but if necessary, buys time and the opportunity for more closely harmonizing the immunological milieu for the transplant organ.

Donor chimerization in a living or deceased donor may be directed to multiple organ donation to the same donor or donation of different organs to different recipients. In the latter circumstance, the adaptive automatic prosthetic disorder response system used to effect chimerization in a living donor and recipient serves to treat the morbidity and sustain homeostasis. Upon death of the donor and identification of the recipient, the system is used to effect chimerization.

When a single recipient is identified, the openings in the surface port empty into the systemie and pulmonary circulation as well as by directly targeted piping to each donor organ, while in organ preparation for transplantation into different recipients, either the port openings are distributed to the different organs, or each organ is provided with its own surface port. The same applies to a body area network emplaced within both donors and recipients, in that intermittently telemetered cross comparison sensor and therapeutic response data is not recipient-coded for a single recipient, but is for multiple recipients, the microcontroller extracting or shredding out only data coded for that recipient, monitors in the center allowing the medical staff to view the data and effect any changes in the existing prescription-programs or regimens.

A direct-to-organ piping means for differentially targeting drugs to different transplant organs allows differential chimerization of different organs in a single donor for distribution to different recipients and differential chimerization of different organs in a single recipient to better match each organ to a different donor. Once the biological materials from the other party have been delivered, the problem for both is selection of the most efficient and effective materials to accommodate the transplant or transplants in as little time as possible.

The means for determining the degree of discrepancy in genome and major histocompatibility complex reasonably tolerable in quantitative abstract terms and not just patch testing, which materials best to use for each organ, the highest rate of safe and effective delivery, the rate of chimerization following targeted delivery of different vehicles for effecting chimerization, such as direct arterial hematopoietic infusion or hepatocytes into the liver, and means for monitoring the rate of chimerization must all be determined. Where targeting seeks to differentially chimerize the different organs for different recipients, even though tightly targeted, chimerizing materials will pass over the venous drainage endothelium and into the systemic and pulmonary circulation.

While it is doubtful that plural or differential organ chimerization in a single donor or recipient would impair overall immune function, the consequences for the overall immunological status of differential organ chimerization in a single donor for whom different recipients must be served or in different recipients for whom donor organs in only different donors can be found must be determined. It is likely that over time, donors and recipients with differentially chimerized organs will settle at some mean immunological status. Then, should one transplanted organ show signs of irremediable rejection, the effect on overall immunological status of the recipient must be ascertained.

Also, retransplantation, where the recipient has been exposed to allogeneic tissue in the past shows less of a propensity for rejection (Savla, J., Lin, K. Y., Pradhan, M., Ruebner, R, L., Rogers, R. S., and 6 others 2015. Op cit.). A reasonable inference is that to chimerize the donor with matter taken from the recipient as well, whether approaching expiration or sustained on life support, prior to removal of the transplant organs would further enhance tolerance in the recipient. The view that multiple organ transplantation is preferable for reasons both of competent organ cooperation and chimerization continues to be refined (see, for example, Lunsford, K. E., Bodzin, A. S., Markovic, D., Zarrinpar, A., Kaldas, F. M., and 7 others 2017. “Avoiding Futility in Simultaneous Liver-kidney Transplantation: Analysis of 331 Consecutive Patients Listed for Dual Organ Replacement,” Annals of Surgery (Philadelphia, Pennsylvania) 265(5): 1016-1024; Choudhury, R. A., Reese, P. P., Goldberg, D. S., Bloom, R. D., Sawinski, D, L., and Abt, P. L. 2017, Op cit.; Cheng, X. S., Stedman, M. R., Chertow, G. M., Kim, W. R., and Tan, J, C. 2017. “Utility in Treating Kidney Failure in End-stage Liver Disease with Simultaneous Liver-kidney Transplantation,” Transplantation 101(5): 1111-1119; Rich, N., Tanriover, B., Singal, A. G., and Marrero, J, A. 2017. “Outcomes of Simultaneous Liver Kidney Transplantation in Patients with Hepatocellular Carcinoma,” Transplantation 101(1): e12-e19; Doyle, M. B., Subramanian, V., Vachharajani, N., Maynard, E., Shenoy, S., Wellen, J. R., Lin, Y., and Chapman, W, C. 2016. “Results of Simultaneous Liver and Kidney Transplantation: A Single-Center Review,” Journal of the American College of Surgeons 223(1): 193- 201; Taner, T., Heimbach, J. K., Rosen, C. B., Nyberg, S, L., Park, W. D., and Stegall, M, D. 2016. “Decreased Chronic Cellular and Antibody-mediated Injury in the Kidney following Simultaneous Liver-kidney Transplantation,” Kidney International 89(4): 909-917; Paterno, F., Girnita, A., Brailey, P., Witte. D., Wang. J., and 9 others 2016. “Successful Simultaneous Liver- kidney Transplantation in the Presence of Multiple High-titered Class I and II Antidonor HLA [human leukocyte antigen] Antibodies,” Transplantation Direct 2(12): e121).

In this circumstance, the defective heart would best be replaced before it can initiate this global cascade of secondary defects. In utero organ transplantation has the advantage of the unique tolerance of the fetus to the allogeneic tissue and ABO incompatibility of its mother (see, for example, Strong, B. S., Newkold, T. J., Lee, A. E., Turner, L. E., Alhajiat, A. M., Heusel, J. W., and Shaaban, A, F. 2016. “Extrinsic Allospecific Signals of Hematopoietic Origin Dictate INKT [invariant natural killer T] Cell Lineage-fate Decisions during Development,” (Nature Research) Scientific Reports 6:28837; Kinder, J. M., Jiang. T. T., Ertelt, J. M., Xin, L., Strong, B. S., Shaaban, A. F., and Way, S. S. 2015. “Tolerance to Noninherited Maternal Antigens, Reproductive Microchimerism and Regulatory T Cell Memory: 60 Years after ‘Evidence for Actively Acquired Tolerance to Rh Antigens’,” Chimerism 6(1-2): 8-20; Alhajiat, A. M., Strong, B. S., Lee, A. E., Tumer. L. E., Wadhwani, R. K., and 3 others 2015. “Prenatal Allospecific N K Cell Tolerance Hinges on Instructive Allorecognition through the Activating Receptor during Development,” Journal of Immunology (Baltimore) 195(4): 1506-1516; Gantt. S., Gervassi, A., Jaspan, H., Horton, H. 2014. “The Role of Myeloid-derived Suppressor Cells in Immune Ontogeny,” Online, Frontiers in Immunology (Lausanne, Switzerland) A5:387; Schminkey, D. L. and Groer, M. 2014. “Imitating a Stress Response: A New Hypothesis about the Innate Immune System's Role in Pregnancy,” Medical Hypotheses 82(6): 721-729; Gammill, H. S., Aydelotte, T. M., Guthrie, K. A., Nkwopara, E. C., and Nelson, J. L. 2013. “Cellular Fetal Microchimerism in Preeclampsia,” Hypertension (Dallas, Texas) 62(6): 1062-1067; Rezaee, F., Peppelenbosch, M., and Dashty, M. 2013, Op cit.; Warning, J. C., McCracken, S. A., and Morris, J. M. 2011. “A Balancing Act: Mechanisms by Which the Fetus Avoids Rejection by the Maternal Immune System,” Reproduction (Cambridge, England) 141(6): 715-724; Gammill, H. S. and Nelson, J. L. 2010. “Naturally Acquired Microchimerism,” International Journal of Developmental Biology 54(2-3): 531-543; Kanellopoulos-Langevin, C., Caucheteux, S. M., Verbeke, P., and Ojcius, D. M. 2003. “Tolerance of the Fetus by the Maternal Immune System: Role of Inflammatory Mediators at the Feto-maternal Interface,” Reproductive Biology and Endocrinology 1:121).

2b(1)(b)(b1). Endothelial Drumming

The extracorporeal master controller is able to synchronize the pulses of donor and recipient with the gradual, incremental, or total advancement, reversal, or total retraction of the diversion chute or chutes. The chutes can be opened, for example, during diastole to have been reset in time for the following systole. To do so adds the higher amplitude onset pulse of the systole to the increased flow rate of the opened diversion chute.

If the blood flow diversion chute is opened from fully closed to the extension specified, added momentum to the synchronous systole, increasing sheer stress against the endothelia of the valve jacket or servochoke substrate vessels and therewith, the probability of somewhat increased rate of uptake of nanoparticles, drugs, and reciprocal cross microchimerizing cellular material between donor and recipient should obtain (see, for example, Jerez, H. E., Simioni, Y. R., Ghosal, K., Morilla, M. J., and Romero, E. L. 2024. “Cholesterol Nanoarchaeosomes for Alendronate Targeted Delivery as an Anti-endothelial Dysfunction Agent,” Online, Beilstein Journal of Nanotechnology (Frankfurt am Main, Germany) 15:517-534; Balakrishnan, B., Kulkami, U. P., Pai, A. A., Illangeswaran, R. S. S., Mohanan, E., and 3 others 2023. “Biomarkers for Early Complications Post Hematopoietic Cell Transplantation: Insights and Challenges,” Online, Frontiers in Immunology (Lausanne, Switzerland) 14:1100306; Deng, Z., Gao, W., Kohram, F., Li, E., Kalin, T. V., Shi, D., and Kalinichenko, V. V. 2023. “Fluorinated Amphiphilic Poly(β-Amino Ester) Nanoparticle for Highly Efficient and Specific Delivery of Nucleic Acids to the Lung Capillary Endothelium,” Online, Bioactive Materials (Beijing, China) 31:1-17; Greenwood, J. C., Talebi, F. M., Jang, D. H., Spelde, A. E., Gordon, E. K., and 9 others 2023. “Low Postoperative Perfused Vessel Density is Associated with Increased Soluble Endothelial Cell Adhesion Molecules during Circulatory Shock After Cardiac Surgery,” Microvascular Research (New York, New York) 150:104595; Hou, L., Zhang, M., Liu, L., Zhong, Q., Xie. M., and Zhao, G. 2023. “Therapeutic Applications of Nanomedicine in Metabolic Diseases by Targeting the Endothelium,” QJM [Quarterly Journal of Medicine] (Oxford, England) 116(7): 493-501; Palomo, M., Moreno-Castaño, A. B., Salas, M. Q., Escribano-Serrat, S., Rovira, M., and 9 others 2023. “Endothelial Activation and Damage as a Common Pathological Substrate in Different Pathologies and Cell Therapy Complications,” Online, Frontiers in Medicine (Lausanne, Switzerland) 10:1285898; Xu. S., Zhang, G., Zhang, J., Liu, W., Wang, Y., and Fu. X. 2023. “Advances in Brain Tumor Therapy Based on the Magnetic Nanoparticles,” Online, International Journal of Nanomedicine (Auckland, New Zealand) 18:7803-7823; Yang Z Y. Yan X C. Zhang J Y, Liang L, Gao C C. and 10 others 2023. “Repression of IRNA [ribosomal rRNA] Gene Transcription by Endothelial SPEN [split end transcription repressor] Deficiency Normalizes Tumor Vasculature via Nucleolar Stress,” Online, Journal of Clinical Investigation (Ann Arbor, Michigan) 133(20): e159860; Yang, Y., Zoulikha, M., Xiao, Q., Huang. F., Jiang, Q., and 3 others 2023. “Pulmonary Endothelium-targeted Nanoassembly of Indomethacin and Superoxide Dismutase Relieves Lung Inflammation,” Online, Acta Pharmaceutica Sinica. B (Amsterdam, North Holland, Netherlands) 13(11): 4607-4620; Zulkapli, R., Muid. S. A., Wang, S. M., and Nawawi, H. 2023. “PCSK9 Inhibitors Reduce PCSK9 [proprotein convertase subtilisin-kexin type 9] and Early Atherogenic Biomarkers in Stimulated Human Coronary Artery Endothelial Cells,” Online, MDPI [Multidisciplinary Digital Publishing Institute] International Journal of Molecular Sciences (Basel, Switzerland) 24(6): 5098; Charo, N., Jerez, H., Tatti, S., Romero, E, L., and Schattner, M. 2022. “The Anti-inflammatory Effect of Nanoarchaeosomes on Human Endothelial Cells,” Online, MDPI [Multidisciplinary Digital Publishing Institute] Pharmaceutics (Basel, Switzerland) 14(4): 736; Moreno-Castaño, A. B., Salas, M. Q., Palomo, M., Martinez-Sanchez, J., Rovira, M., and 7 others 2022. “Early Vascular Endothelial Complications after Hematopoietic Cell Transplantation: Role of the Endotheliopathy in Biomarkers and Target Therapies Development,” Online, Frontiers in Immunology (Lausanne, Switzerland) 13:1050994; Nong. J., Glassman, P. M., and Muzykantov, V, R. 2022. “Targeting Vascular Inflammation through Emerging Methods and Drug Carriers,” Online, Advanced Drug Delivery Reviews (Amsterdam, North Holland, Netherlands) 184:114180; Reschke, M., Piotrowski-Daspit, A. S. Pober, J. S., and Saltzman, W. M. 2022. “Nucleic Acid Delivery to the Vascular Endothelium,” Online, [American Chemical Society] Molecular Pharmaceuties (Washington, DC) 19(12): 4466-4486; Lia, G., Giaccone, L., Leone, S., and Bruno, B. 2021. “Biomarkers for Early Complications of Endothelial Origin after Allogeneic Hematopoietic Stem Cell Transplantation: Do They Have a Potential Clinical Role?,” Online, Frontiers in Immunology (Lausanne, Switzerland) 12:641427; Glassman, P. M., Myerson, J. W., Ferguson, L. T., Kiseleva, R. Y., Shuvaev, V. V., Brenner, J. S., and Muzykantov, V, R. 2020. “Targeting Drug Delivery in the Vascular System: Focus on Endothelium,” Online, Advanced Drug Delivery Reviews (Amsterdam, North Holland, Netherlands) 157:96-117; Lysyy. T., Bracaglia, L. G., Qin, L., Albert, C., Pober, J. S., and 3 others 2020. “Ex vivo Isolated Human Vessel Perfusion System for the Design and Assessment of Nanomedicines Targeted to the Endothelium,” Online, Bioengineering and Translational Medicine (Hoboken, New Jersey) 5(2): e10154; Mohd Ariff, A., Abu Bakar, N. A., Abd Muid, S., Omar, E., Ismail, N. H., and 3 others 2020. “Ficus deltoidea Suppresses Endothelial Activation, Inflammation, Monocytes Adhesion and Oxidative Stress via NF-κB [nuclear factor-κB] and eNOS [endothelial nitric oxide synthase] Pathways in Stimulated Human Coronary Artery Endothelial Cells,” BioMed Central Complementary Medicine and Therapies (London, England) 20(1): 56; Schnitzler, J. G., Dzobo, K. E., Nurmohamed, N. S., Stroes, E. S. G., and Kroon, J. 2020. “Surmounting the Endothelial Barrier for Delivery of Drugs and Imaging Tracers,” Online, Atherosclerosis (Limerick, Ireland) 315:93-101; Kiseleva, R. Y., Glassman, P. M., Greineder, C. F., Hood, E. D., Shuvaev, V. V., and Muzykantov, V, R. 2018. “Targeting Therapeutics to Endothelium: Are We There Yet?,” Drug Delivery and Translational Research (New York, New York) 8(4): 883-902; Myerson, J. W., Braender, B., Mcpherson, O., Glassman, P. M., Kiseleva, R. Y., and 9 others 2018. “Flexible Nanoparticles Reach Sterically Obscured Endothelial Targets Inaccessible to Rigid Nanoparticles,” Online, Advanced Materials (Baden-Württemberg, Germany) 30(32): e1802373; Reijers, J, A. A., Dane, M. J. C., van Zonneveld, A. J., Burggraaf. J., and Moerland, M. 2018. “Potential Influence of Endothelial Adsorption on the Delayed Time to Maximum Concentration of Biopharmaceuticals,” Online, European Journal of Drug Metabolism and Pharmacokinetics (Paris, France) 43(1): 103-113; Qiu Y., Tong, S., Zhang, L., Sakurai, Y., Myers, D. R., and 3 others 2017. “Magnetic Forces Enable Controlled Drug Delivery by Disrupting Endothelial Cell-Cell Junctions,” Nature Communications (London, England) 8:15594; Shuvaev, V. V., Brenner, J. S., and Muzykantov, V, R. 2015. “Targeted Endothelial Nanomedicine for Common Acute Pathological Conditions,” Online, Journal of Controlled Release 219:576-595; Su, J, B. 2015. “Vascular Endothelial Dysfunction and Pharmacological Treatment,” Online, World Journal of Cardiology (Pleasanton, California) 7(11): 719-741; Voigt, J., Christensen, J., and Shastri, V. P. 2014. “Differential Uptake of Nanoparticles by Endothelial Cells through Polyelectrolytes with Affinity for Caveolae,” Online, Proceedings of the National Academy of Sciences of the United States of America (Washington, D C) 111(8): 2942-2947; Muzykantov, V. R., Radhakrishnan, R., and Eckmann, D. M. 2012. “Dynamic Factors Controlling Targeting Nanocarriers to Vascular Endothelium,” Online, Current Drug Metabolism (Boca Raton, Florida) 13(1): 70-81; Michell, D, L., Andrews, K, L., Woollard, K. J., and Chin- Dusting, J. P. 2011. “Imaging Leukocyte Adhesion to the Vascular Endothelium at High Intraluminal Pressure,” Online, Journal of Visualized Experiments (Boston, Massachusetts) (54): 3221; Simone, E., Ding, B. S., and Muzykantov, V. 2009. “Targeted Delivery of Therapeutics to Endothelium,” Online, Cell and Tissue Research (Berlin, Germany) 335(1): 283-300; Aird, W, C. 2005. “Spatial and Temporal Dynamics of the Endothelium,” Journal of Thrombosis and Haemostasis (New York, New York) 37): 1392-1406; Melzig, M. and Teuscher, E. 1987. “Interaction of Drugs and the Adenosine-uptake System of Endothelial Cells May Influence the Regulation of the Heart by Adenosine,” Biomedica Biochimica Acta (Berlin, Germany) 46(8-9): S650-S652).

Localized increase in sheer stress and/or pressure can also be used to increase the targeted uptake of drugs, which can be further facilitated through the application of a magnetized periductal jacket and a drug formulated as a magnetically susceptible nanoparticulate, as well as through endothelial drumming, or thumping. Magnetized jackets to target uptake along a circumscribed segment along a ductus are described and illustrated in U.S. Pat. No. 11,389,171, entitled Integrated System for the Infixion and Retrieval of Implants. By contracting on diastole and opening on systole, a choke servovalve such as that diagrammatically depicted in FIG. 11 of U.S. Pat. No. 11,759,186, entitled Ductus Side-entry Jackets and Prosthetic Disorder Response Systems, can be used to locally increase pressure and shear stress upon release and the reverse in the same way.

Magnetized jackets positioned downstream near to the diversion or choke servovalve can be used for the purpose of increasing segment-targeted drug uptake over and above dependency upon endothelial drumming. An additional method, combinable with the others, places a choke servovalve jacket, or electrical sphincter, ahead of (downstream) a conventional or diversion chute side-entry jacket so that contraction thereof increases the force of flow over the endothelium, or in a urinary application, the urothelium. Whether used apart from or in synchrony with diversion of choke servovalves that open on systoles and close on diastoles to reinforce the uptake of drugs when prepared in the form of superparamagnetic nanoparticles, magnet jackets are generally permanent and magnetized radially at right angles to their longitudinal central axis.

Such magnets are not variable as are electromagnet jackets to vary the field strength as yet another variable to apply in synchrony to further boost uptake over and above the static field strength. To accomplish omnidirectional periductal attractive force using electromagnets, which are unidirectional, thereby gaining the further advantage of variability in field strength is practicable where a lesion to be treated is asymmetrical, but results in the need for multiple magnets for positioning in a very small space, resulting in excessive size and weight for application along the arterial tree.

In metered compound vascular bypass, or sudden switched-organ transplantation, if the immune response necessitates, the master control microprocessor can fully retract the diversion chutes in the servovalves to the fully retracted, closed position from any degree of extension, thereby isolating the bloodstreams of donor and recipient making these independent with no blending of bloodstreams. Immunosuppressives released as appropriate, the controller can wait out the adverse reaction and then resume the process. If necessary, a slow heparin or heparinoid drip prevents the accumulation of clot through the valves. While apportioned between the two, the total volume of blood exchanged between donor and recipient is constant. Hypothetically, should an adverse immune reaction necessitate, valves connecting different like vessels can be adjusted differently.

Analogous to the opening and closing of a floodgate, to which the bloodstream adds pulsation, the transient increase in blood pressure translates into increased shear stress and flow rate to assist the transit of blood between donor and recipient. Accordingly, the frequency, pattern, and duration of the switching of each valve pair, one per vessel in the donor and recipient, are automatically controlled by the extracorporeal master control unit to which the microprocessor or master controller implanted within and responsible for either participant is subordinated so long as the two circulatory systems are connected. The servovalves can be moved from fully or partially open to fully closed repeatedly, for example.

There is therefore an alteration in direct control between extracorporeal master controller during reciprocal cross-circulation or cross-infusion, and relegation to the implanted microprocessors respective of donor and recipient when the valves are closed to stop reciprocal cross-circulation. Increased pressure in the forward or antegrade direction is obtained by opening the valves just after the diastolic wave passes, while increased pressure in the backward or retrograde direction is obtained by stopping the diastolic wave on arrival. When the valves close, the abrupt stoppage of antegrade flow through the valve to the opposite party results in increased pressure backward rebound within the respective circulatory systems which likewise increases the shear stress against the endothelium, again serving to facilitate uptake.

The weaker pulse in veins makes these effects weaker. In this way, intermittent or alternating switching between connection to a variable extent and disconnection of the two circulatory systems to cause transient increases in the blood forward and back pressure can be used to achieve increased cross-microchimerization and line-transmitting propulsive force. Switching between donor and recipient independent and shared circulation to facilitate immune tolerance induction is unknown, hence, untested; the relative effectiveness of intermittent blood exchange and the rate and pattern of switching as opposed to a continuous gradual transfer of circulation from the recipient to the donor warrant investigation. Heart, lung, and heart-lung transplantation are addressed below.

### Supraparamagnetic Iron Oxide Nanoparticle Traps

Closely related to the concept of endothelial drumming is that the supraparamagnetic (or superparamagnetic) iron oxide nanoparticle traps, which use stent- or impasse-jackets as described in U.S. Pat. No. 11,389,171 to increase endothelial uptake with or in the absence of drumming.

2b(1)(b)(b2). Prenatal Compound Vascular Bypass, or Switched-Organ Transplantation of the Heart

The sooner a heart so congenitally defective that surgery could never repair it to the extent that it would function normally the better notwithstanding, prenatal compound vascular bypass, or switched-organ transplantation of the heart should take place only later in gestation with a donor heart of the same or no more than slightly greater age. This is because to place a more highly developed heart with an ejection fraction disproportionately high in relation to the rest of the vascular tree would soon prove injurious. Moreover, “Preterm hearts differ significantly from a term neonate's heart, and there is a gradual transition to a mature neonate heart,” (see, for example, Abushaban, L., Mariappa, T. V., Rathinasamy, J, and Sharma, P. N. 2018. “Normal Reference Ranges for Aortic Diameters in Preterm Infants,” Online, Journal of the Saudi Heart Association (Riyadh, Saudi Arabia) 30(2): 86-94).

In prospective late prenatal, neonatal, infant, and toddler compound bypass heart transplantation, grouped vessel inlet/outlet confluence conduits as shown in FIGS. 15C thru 15E addressed below in section 2b(1)(b)(b14), entitled Confluence Conduits: Connection of One or More Bloodlines to Flow through a Common Channel to the Same or a Different Number of Bloodlines with Self-leveling Apportionment of Outflow, in the subsection thereof entitled Tiny Vessel Grouped Inlet/Outlet Expandable Confluence Conduits, and in section 6, entitled Description of the Preferred Embodiments of the Invention, to significantly reduce the duration of the operation for the already impaired patient thus improving the likelihood of a favorable outcome.

Provided the disproportion is less pronounced and the alternative is to allow the prospective neonate to die upon or shortly following birth, negative inotropes to include beta-blockers, calcium channel blockers, and antiarrhythmics such as atenolol, cibenzoline, clonidine, disopyramide, flecainide, itraconazole, metoprolol, nifedipine, sunitinib, and verapamil should be used to bring down the increase in blood pressure. Using currently conventional methods, the youngest patient able to undergo a heart transplant is a neonate (see, for example, John, M. and Bailey, L. L. 2018 Op cit. in section 2a, entitled Background of the Invention. General); (see also Schumacher, K. R., Almond, C., Singh, T. P., Kirk, R., Spicer, R., and 7 others 2015. “Predicting Graft Loss by 1 Year in Pediatric Heart Transplantation Candidates: An Analysis of the Pediatric Heart Transplant Study Database,” Online, Circulation (Hagerstown, Maryland) 131(10): 890-898); Thrush, P. T. and Hoffman, T. M. 2014. “Pediatric Heart Transplantation. Indications and Outcomes in the Current Era,” Online, Journal of Thoracic Disease (Hong Kong, China) 6(8): 1080-1096).

The trauma inflicted by a conventional heart transplant, which is considerably greater in extent than that imposed by the compound vascular bypass method, would be prohibited in a fetus, especially one congenitally impaired and frail, leaving a compound vascular bypass transplant as the only permissible method for achieving a favorable outcome. If so, then compound bypass heart transplantation will have been the difference that makes such a heart transplant possible, likely interdicting all or almost all of the global degradation that poor circulation would otherwise have caused.

Unfortunately, however, not all of the trauma in a heart transplant can be eliminated. The incision to gain entry into the chest cannot be avoided, and the minimal automatic disorder monitoring and response system needed in the fetus to administer the transplant even though placed laparoscopically while little traumatic or causing pain that lasts once healed compared to a chest incision which can cause post-healing pain for years, is likewise unavoidable. Nonessential automatic system components are not placed in the patient until well after delivery.

The fully implanted disorder response system is implanted in the gravida and placenta as soon as the condition of defective development is detected, addressed in sections 2a, entitled Background of the Invention. General, 2a(2), entitled Inventive Content-pertinent Fetal Diagnostics. Later in gestation, only those automatic disorder response system components necessary to administer the transplant such as indispensable sensors, bloodlines, druglines, servovalves, and master control microprocessor are implanted in the fetus. Placement is least intrusive using laparoscopic techniques to the extent possible.

During this initial period, to implant the monitoring and response functions that in an adult would have been provided at the outset are deferred to allow the baby to gain in strength. Until then, functions not implanted are provided exogenously in the clinic. Following the newborn stage, additional components to monitor and respond to the condition of the graft organ such as additional sensors and drug reservoirs are entered laparoscopically with the prescription-program updated accordingly.

Only components that will not require replacement, such as the master control microprocessor, essential sensors, and drug delivery components are implanted. The minimization if not the elimination of a need for follow-up operations, even a few years later, is a central consideration in the measures taken early in the process. To accommodate growth, druglines and bloodlines are made in accordance with 2b(2)(f), entitled Expandable Structure to Accommodate Growth and Self-adjust in Size in Vascular Segment Prostheses, Lines, and Confluence Conduits Placed in Children.

Due to the minute size of the vessels in a neonate much less a prenate, the expense of the numerous micromachined valves, each with microminiature actuators needed to perform a compound bypass heart transplant, the time needed to separately connect each of the numerous vessels to a valve, and the operational duration and stress imposed on the patient are much reduced (see, for example, Akturk, Y. and Ozbal Gunes, S. 2018. “Normal Abdominal Aorta Diameter in Infants, Children, and Adolescents,” Pediatrics International (Carleton South, Victoria, Australia) 60(5): 455-460; Machii, M. and Becker, A, E. 1997. “Morphologic Features of the Normal Aortic Arch in Neonates, Infants, and Children Pertinent to Growth,” Online, Annals of Thoracic Surgery (Amsterdam, North Holland, Netherlands) 64(2): 511-515).

Addressed in the subsection to follow and shown in FIGS. 15C and 15D, this is made possible with the aid of combined vessel diversion chute-pass confluence conduits, or shared diversion chute multiple vessel inlet and outlet confluence conduits. Using these, flow through the vessel inlets into and outlet from the confluence conduit is converged upon entering and diverged exiting the conduit. The vessels placed in adjacent or bunched relation with the outflow from the inlet vessels are grouped to flow together through or past a common diversion chute which splits the column between one fraction that is diverted into the confluence conduit and one which continues undiverted.

When the inlet and outlet diversion chutes are driven by a bistable plunger solenoid, all flow is switched between complete diversion and nondiversion. When driven by a servomotor as is essential to perform a metered compound vascular bypass transplant, control over the relative proportion of the fractions is continuously variable and responsive in rate and direction to rejection sensor feedback to the master control microprocessor. The quicker and less stressful method advocated for prenates and neonates being the sudden switch, the shared input vascular valve would be driven by a linear plunger solenoid.

Especially in pediatric compound vascular bypass solid organ transplantation, because it obstructs the continuation of pulsatile flow much as does nonpulsatile Fontan circulation resulting in impaired development, an inline axial pump in a switchable bloodline as described below in section 2b(1)(b)(b15), entitled Switchable Lines and Confluence Conduits: Long when Intercorporeal, to Short when Intracorporeal, Both Affording Connection of One or More Bloodlines, and shown in FIGS. 32A thru 32C must be limited to the longer of the two bloodlines, that longer discarded leaving that shorter to be implanted.

Such a pump can assist in propelling the blood over the longer course while reducing or eliminating the need for both the deceased donor and recipient to be placed on a positive inotrope, thus eliminating the potential for serious adverse complications. “While inotropes successfully increase cardiac output, their use has been plagued by excessive mortality due to increased tachycardia and myocardial oxygen consumption leading to arrhythmia and myocardial ischemia,” (Francis, G. S., Bartos, J. A., and Adatya, S. 2014, Op cit., above in section 2a, entitled Background of the Invention. General).

The same in mechanism as switchable bloodlines, the longer of the two conduits must abide by the same proviso. Whereas the longer conduit can be a simple tube into which the pump is easily positioned, the shorter of the two omits an inline pump and should be made as described below in section 2b(2)(f), entitled Expandable Structure to Accommodate Growth and Self-adjust in Size in Vascular Segment Prostheses, Lines, and Confluence Conduits Placed in Children. With both switchable bloodlines and switchable confluence conduits, both the longer and shorter lines are provided with at least one drugline 8 and 8′, available for releasing immune tolerance inducing or immunosuppressant drugs, for example. All lines or conduits best incorporate a thin film rejection sensor cemented along the internal surface.

If clinical judgment is that the metered method is indicated, the diversion chute would be driven by a servomotor. While the need for the metered switch method in a prenate or neonate would likely be justified only in a limited number of cases, using the metered switch method, the outflow of the bunched vessels is divided between that the fraction diverted and the fraction which continues to flow past the diversion chute. Switchable combined vessel diversion chute-pass confluence conduits relate to that singular shown in FIG. 35A as do combined vessel diversion chute-pass confluence conduits to that singular shown in FIG. 35B.

In FIGS. 15C and 15D, when driven by a plunger solenoid, the diversion chute seen on the left-hand side at the entry into the conduit makes possible a sudden switch organ transplant. When driven by a servovalve, the organ transfer can be metered. In an older patient with a mature immune system, this is beneficial in providing an interval for microchimerization during the donor-recipient reciprocal cross-circulation used to transfer the graft organ from the circulatory system of the donor and into that of the recipient depicted in FIG. 15 and in avoiding the shock of stark confrontation with alien tissue.

The late prenate or neonate patient having been matched to the recipient, and if necessary, immune tolerance medication having been administered, the immature and relatively weak immune system of the patient and the desideratum to minimize the duration of the procedure and stress on the patient recommend taking advantage of the sudden switch technique. However, if the patient is considered robust enough, use of the metered switch technique is always preferrable. In a sudden switch transplant, the implanted valves on each bloodline connecting corresponding vessels of the donor and recipient can be manually actuated by the operator from the same switch, an implanted microprocessor used for follow-up monitoring and medicinal support of the graft organ.

In a metered transplant, the implanted master control microprocessor will control the vascular valves or servovalves to administer the transfer of the graft organ from the circulatory system of the donor into that of the recipient by pacing this action on the basis of rejection reaction sensor inputs to modulate the rate of transfer until it is completed. Thereafter, it will continue to monitor the progress of fetal development as well as automatically site-target essential medication to include immune tolerance agents if necessary. The fetus returned to the uterus, the mode of delivery is entrusted to the obstetric staff.

Yet to become a reality, prenatal heart transplantation is not addressed in the literature. Related subjects such as childbearing after a heart transplant are well covered. If the heart transplant had been performed using the metered compound bypass technique and the recommendation to retain the monitoring and treatment components of the implanted automatic disorder response system implemented in order to allow the automatic therapeutic direct pipe-targeting of pharmaceuticals responsive to signals from the sensors to continue postoperatively, the improved health of the gravida would be better prepared to cope with a pregnancy and delivery. The same applies when the gravida had been implanted with an automatic disorder response system to monitor and treat any chronic disease such as diabetes or an inborn error of metabolism.

This factor should reduce the risks and improve the outcomes compared to delivery following a conventional heart transplant which is well documented in the literature to indicate the serious complications which would otherwise have gone without the amelioration if not complete suppression prior to the pregnancy that the automatic response system provided. Accordant with the objects of the invention specified below in section 4, the measures delineated herein could well render a patient otherwise incapable of pregnancy and delivery capable of childbearing. The system can not only respond to the medical needs of the patient but administer the transplant.

Prior to pregnancy, the implanted automatic disorder response system whatever disorder the prospective gravida has. “The post-cardiac transplant patient is at a higher risk of complications both during pregnancy and in the postpartum period. Risks include hypertension, pre-eclampsia, eclampsia, infection, gestational diabetes, and thromboembolic disease, all of which can lead to adverse maternal, fetal, and neonatal outcomes,” (Abdalla, M. and Mancini, D. M. 2014. “Management of Pregnancy in the Post-cardiac Transplant Patient, Online, Seminars in Perinatology (Philadelphia, Pennsylvania) 38(5): 318-325).

Here limited to heart transplantation, comparable references could be provided for the earlier transplant of any other major organ or combination thereof (see, for example, AlRaffa., A, AlJubairy, S., and Alwatban, S. J. 2023. “A Successful Pregnancy and Delivery after Heart Transplantation: The First Case Report in Saudi Arabia,” Online, Saudi Journal of Anaesthesia, (Mumbai [Bombay], India) 17(1): 97-100; Craig, A. M., Campbell, A., Snow, S. C., Spates, T. N., Goldstein S A, and 7 others 2023. “Maternal and Pregnancy Outcomes following Heart Transplantation in the United States,” Journal of the American College of Cardiology. Heart Failure (New York, New York) 11(12): 1666-1674; DeFilippis, E. M. and Kittleson, M. M. 2023. “Pregnancy as a Sentinel Event: Preventing Severe Maternal Morbidity in Heart Transplant Recipients,” Journal of the American College of Cardiology. Heart Failure (New York, New York) 11(12): 1675-1677; Irani, R. A., Coscia, L. A., Chang, E., and Lappen, J. R; 2023. “Prepregnancy Evaluation and Pregnancy Management of Patients with Solid Organ Transplants,” Online, American Journal of Obstetrics and Gynecology (New York, New York) 229(2): B10-B32; Boyle, S., Sung-Him Mew, T., Lust, K., Mckenzie, S., Javorsky, G., and Parsonage, W. 2021. “Pregnancy following Heart Transplantation: A Single Centre Case Series and Review of the Literature,” Online, Heart, Lung, and Circulation (Chatswood, New South Wales, Australia) 30(1): 144-153; Defilippis, E. M. and Kittleson, M. M. 2021. “Pregnancy after Heart Transplantation,” Journal of Cardiac Failure (Philadelphia, Pennsylvania) 27(2): 176-184; Acuna, S. Zaffar, N., Dong, S., Ross, H., and D'Souza, R. 2020. “Pregnancy Outcomes in Women with Cardiothoracic Transplants: A Systematic Review and Meta-analysis,” Journal of Heart and Lung Transplantation (New York, New York) 39(2): 93-102; Punnoose, L. R., Coscia, L. A., Armenti, D. P., Constantinescu, S., and Moritz, M. J. 2020. “Pregnancy Outcomes in Heart Transplant Recipients,” Online, Journal of Heart and Lung Transplantation (New York, New York) 39(5): 473-480; Pais, A. S., Guerra, N., Couto, D., Sousa, A. P., and Almeida-Santos, T. 2019. “Fertility Preservation with Successful Pregnancy Outcome in a Patient with Transplanted Heart and Non-Hodgkin's Lymphoma—A Case Report,” Online, BioMed Central Pregnancy and Childbirth (London, England) 19(1): 421).

Enumerated above in Section 2a are numerous means for avoiding the protrusion of vascular valve and servovalve sidestems into neighboring tissue. The process will be simplified through the application of confluence conduits as addressed in sections 2b(1)(b)(b14), entitled Confluence Conduits: Connection of One or More Bloodlines to Flow through a Common Channel to the Same or a Different Number of Bloodlines with Self-leveling Apportionment of Outflow, shown in FIG. 35A, and 2b(1)(b)(b15), entitled Switchable Lines and Confluence Conduits: Long when Intercorporeal, to Short when Intracorporeal, Both Affording Connection of One or More Bloodlines to the Same or a Different Number of Bloodlines with Self-leveling Apportionment of outflow, shown in FIG. 35B.

While surgery on a human fetus was first reported in 1963, neonatal heart transplantation has become a reality while fetal (prenatal, antenatal, in utero) heart transplantation, addressed below in this section, remains unreported (O'Connor, K., and O'Neil, E. 2012. “Fetal Surgery, in The Embryo Project Encyclopedia, Tempe, Arizona: Arizona State University), then in veterinary subjects in 1965 (Koehler, S. M., Knezevich, M., and Wagner, A. 2017. “The Evolution of Fetal Surgery,” Online, Journal of Fetal Surgery 1(1): 07-23; (see also Evans, L. L. and Harrison, M, R. 2021. “Modern Fetal Surgery—An Historical Review of the Happenings that Shaped Modern Fetal Surgery and Its Practices,” Online, Translational Pediatrics 10(5): 1401-1417; Sala, P., Prefumo, F., Pastorino, D., Buffi, D. Gaggero, C. R., Foppiano, M., and De Biasio, P. 2014. “Fetal Surgery: An Overview,” Obstetrical and Gynecological Survey (Baltimore, Maryland). (69(4): 218-228; Wenstrom, K, D. and Carr, S, R. 2014 “Fetal Surgery: Principles, Indications, and Evidence,” Obstetrics and Gynecology 124(4): 817-835; Adamsons, K. Jr., Freda, V. J., James, L. S., and Towell, M, E. 1965. “Prenatal Treatment of Erythroblastosis Fetalis following Hysterotomy,” Pediatrics 35:848-855), and later in 1993 (Harrison, M. R., Adzick, N. S., Flake, A. W., Jennings, R. W. Estes. J. M., and 9 others 1993. “Correction of Congenital Diaphragmatic Hernia in Utero: VI. Hard-earned Lessons,” Journal of Pediatric Surgery 28(10): 1411-1418).

That a prenatal heart transplant would be completely extracardiac, meaning without incision into either the donor or the recipient heart, makes the procedure less complex as well as fundamentally less traumatic for the graft organ and the fetus. Simpler procedures that will be pertinent to a fetal heart transplant such as a blood transfusion to ameliorate erythroblastosis fetalis have been possible during gestation for over 60 years. (see, for example, Nassar, G. N. and Wehbe, C. 2022. “Erythroblastosis Fetalis,” Online, Treasure Island, Florida: StatPearls Publishing Co.; Adamsons, K. Jr., 1966. “The Role of Hysterotomy and Prenatal Exchange Transfusion in the Management of Severe Erythroblastosis Fetalis,” Bibliography of Gynaecology 38:160-168; Adamsons, K. Jr., Freda, V. J., James, L. S., and Towell. M, E. 1965. “Prenatal Treatment of Erythroblastosis Fetalis following Hysterotomy,” Pediatrics 35:848-855; Dawes, G. S. 1965. “Prospects in Prenatal Physiology, Diagnosis, and Treatment,” Pediatrics 35:731-732; Corston, J. M., Morton, B S., and Robinson, S, C. 1964. “Intrauterine Blood Transfusion of the Fetus,” Online, Canadian Medical Association Journal 91(15): 815-817; Liley, A. W. 1963. “Intrauterine Transfusion of Foetus in Haemolytic Disease,” Online, British Medical Journal 2(5365): 1107-1109).

Because it materially reduces trauma and eliminates ischemia-reperfusion injury, provided such a procedure is genetically justified, the advent of compound vascular bypass, or switched-organ transplantation should not only make heart transplantation in a prenate possible but avoid the factors that lead to less than desired outcomes in neonates. The earlier in gestation is the need for a heart transplant indicated, the less developed is the native heart so that were the replacement heart harvested at the same stage in development as that native, the longer would be the period for development of the replacement heart in its new body. This suggests that the graft organ would best be farther along in development than was the native heart.

Vascular valves and servovalves configured as shown in FIGS. 2, 5, 7, 8, and 10A can be positioned anywhere along the length of a ductus-usually a blood vessel or a ureter—and rotated to any angle at that level. In microminiature sizes, this should allow accommodation without protrusion of the side-stem into neighboring tissue in very small patients such as prenates, neonates, and small laboratory animals, where these degrees of freedom should allow an irritation-free fit along almost any ductus. Numerous means for avoiding protrusion into neighboring tissue are delineated above in section 2a, entitled Background of the Invention. General. Until further development in hydraulically amplified self-healing electrostatic, or HASEL actuators when the function of a linear actuator can be duplicated, the protrusive part of valves and servovalves for use in small patient will require effective padding.

The need for normal circulation begins five sixths of the time the fetus has yet to remain in utero—this to support the internal development of the fetus concurrent with the umbilical blood supply and drainage which sustains the fetus as a whole. A central concern is whether the malformity of the native heart had been due to pleiotropic genes that made not only the heart but its supporting physiology and anatomy defective as well leading to collateral defects, so that normal graft would soon develop abnormally as well.

For this reason, the medical geneticist should assist in ascertaining the most likely outcome for a transplant. Genetic defects of the heart are addressed above in sections 2a, entitled Background. General, and 2a(1), entitled Background. Congenital Defects of the Heart and Other Organs—the Importance of Prompt Intervention, and neonatal heart transplantation is dealt with in the following section, 2b(1)(b)(b3), entitled Neonatal and Infant Compound Vascular Bypass, or Switched-organ Transplantation of the Heart.

If the malformity of the heart is secondary to primary congenital or acquired defects in the rest of the body, it must be determined whether more than just the heart must be replaced, and/or exogenous endocrinological and/or enzymatic support must also be provided, or whether instead the malformed heart would best be treated conventionally by allogeneic tissue repair as well as medicinal support. Otherwise, prenatal heart transplantation would be of benefit because the extracardiac development of the embryo contingent upon normal circulation begins well before delivery. The main hindrances to compound vascular bypass heart transplantation in prenates is the tiny size of the vessels, hence the servovalves, and the specialized training in microsurgery needed to perform such operations.

Due to the immaturity of the immune system during the early stage of life, the sudden switch technique would likely be used more often; however, the immaturity of the prenatal immune system and the system of the mother effective notwithstanding, maternal and paternal chimerism and various genetic aberrations might well justify use of the gradual, or metered, technique. As it stands, once a baby is bom with a malformed heart, the outcome will be adversely affected. That is, there will be no completely good way out-if the surgery is flawless, the child will have survived but incurred neurological and/or psychological deficits that will present as inadequate performance in school and the use of systemic immunosuppressives for life will likely shorten that life.

To the extent that the considerable trauma of the surgery, the use of life support, general anesthesia, and cardioplegia are responsible for such neurological deficits, it is posited that to eliminate all of these risk laden measures through the use instead of metered compound vascular bypass, or switched-organ transplantation might well eliminate these adverse outcomes as well. Beginning with the earliest fluttering of the embryo heart around the sixth week into gestation, normal circulation is essential for the continuation of normal development, and required to and throughout adulthood to the end of life, normal pulsatile circulation is essential for the sustainment of normal bodily and immunological function. Also sought is the ability where generically appropriate to transplant the heart as soon as possible-if clinical judgment acquiesces, in utero.

Reciprocally, a breakdown in the normal structure of the heart and vessels in the adult—usually due to a combination of inadequate exercise, a poor diet, and often the excessive consumption of alcohol, inappropriate drugs, and/or inhaling cigarette smoke, together with a genetic component—is accompanied by a gradual breakdown in normal cardiovascular and pulmonary structure, which—unless the mother had been exposed to teratogenic substances in the environment or engaged in alcohol consumption, for example—would have allowed normal development in the first place.

With the advent of echocardiography, or ultrasonography, and cardiotocography, as well as other means for imaging the fetus to detect and counteract more severe deviations from normal development of the heart, potential gravidae should have their fetus examined in detail. Such defects range from the long QT syndrome through the administration of potassium and a beta blocker if not placement of an implantable pacemaker/cardioverter defibrillator to Brugada syndrome, to a univentricular heart with which only a heart transplant would allow the instatement of normal pulsatile circulation (see, for example, El Sayed, M., Goyal. A., and Callahan, A. L. 2023. “Brugada Syndrome,” Online, Treasure Island, Florida: StatPearls Publishing Co.; Li, K, H. C., Lee. S., Yin. C., Liu, T., Ngarmukos, T., and 5 others 2020. “Brugada Syndrome: A Comprehensive Review of Pathophysiological Mechanisms and Risk Stratification Strategies,” Online, International Journal of Cardiology, Heart, and Vasculature (Shannon, Ireland) 26:100468).

While prenatal heart transplantation remains to become a practical reality, prenatal surgery, even cardiac to reconstruct malformities using autogenous tissue, has made headway, as have the correction of other serious malformities such as spina bifida, meningomyelocele (or myelomeningocele), congenital aortic stenosis, and obstructive uropathy (see, for example, Bergh, E., Buskmiller, C., and Johson, A. 2021. “The Future of Fetal Surgery,” Obstetrics and Gynecology Clinics of North America 48(4): 745-758; Evans, L. L. and Harrison, M, R. 2021. Op cit. above in this section; Sampat. K. and Losty, P, D. 2021. “Fetal Surgery,” Online, British Journal of Surgery 108(6): 632-637; Sharma, S. 2019. “Progress in Fetal Surgery: A Buoyant Start of Watchful Reluctance? Perspective for India,” Online, Journal of the Indian Association of Pediatric Surgeons 24(1): 1-3 Maselli, K. M. and Badillo, A. 2016. “Advances in Fetal Surgery,” Online, Annals of Translational Medicine 4(20): 394; Kahn, L., Mbabuike, N., Valle-Giler, E. P., Garces, J., Moore, R.iC., St. Hilaire, H., and Bui, C. J. 2014. “Fetal Surgery: The Ochsner Experience with in Utero Spina Bifida Repair,” Online, Ochsner Journal 14(1): 112-118; Sala, P., Prefumo, F., Pastorino, D., Buffi, D., Gaggero, C, R. Foppiano, M., and De Biasio, P. 2014. “Fetal Surgery: An Overview,” Online, Obstetrical and Gynecological Survey 69(4): 218-228; Wenstrom, K, D. and Carr, S, R. 2014, Op cit., above in this section; Adzick, N. S. 2013. “Prospects for Fetal Surgery,” Online, Early Human Development 89(11): 881-886; Diemert, A., Diehl, W., Glosemeyer, P., Deprest. J., and Hecher, K. 2012. Intrauterine Surgery—Choices and Limitations,” Online, Deutsches.Ärzteblatt International (Cologne, Germany) 109(38): 603-638; Wojcicki, P. and Drozdowski, P, H. 2011. “In Utero Surgery-Current State of the Art,”-Part I, Online, Medical Science Monitor 16(11): RA237-RA244; Part II. Wojcicki, P., Drozdowski, P. H., and Wojcicki, K., Online, Medical Science Monitor 17(12): RA262-RA270; Farmer, D. 2003. “Fetal Surgery,” Online, British Medical Journal 326(7387): 461-462; Harrison, M, R. 1993. “Fetal Surgery,” Online, Western Journal of Medicine 159(3): 341-349).

The advancement of obstetrics may be expected to include ex utero intrapartum treatment, or ‘EXIT’ procedures, beyond the interdiction of airway obstructions which was its initial inducement, so that the detection of a significantly defective solid organ will be responded to by its replacement ex utero. And because its normal function is essential for all other normal development, the heart will certainly mandate ex utero (intrapartum, antepartum, prenatal, fetal) heart transplantation. Because it is critically lower in trauma at this critical stage in development, the compound vascular bypass method should prove essential to even make possible a heart transplant at this frail stage, and should supplant any alternative technique for transplantation of the heart in any patient for any reason.

While posing the additional problem of having to temporarily remove the fetus from and then return it to the uterus, the fetal heart poses offsetting reduction in complexity in that relatively undifferentiated, the number of reciprocal cross-circulatory connections is less and the immune system is less highly developed and therefore less likely to pose resistance to the acceptance of the new heart. Yet another constraint, a severe limitation in the pool of available graft organs can be avoided. The legalization of abortion in several states has made available graft organs that previously would have been discarded. With viable fetuses otherwise to be destroyed in any event, organs needed can be sustained in utero until transplanted.

In the United States, well over six hundred thousand viable fetuses are intentionally destroyed every year. Where allowed, the disposition of these fetuses as a source of replacement organs and tissues should be made a matter of law, with the maintenance of such fetuses substantially the same as previously specified for brain dead adults (see, for example, Kortsmit, K., Mandel, M. G., Reeves, J, A. Clark, E., Pagano, H. P., and 3 others 2021, “Abortion Surveillance—United States, 2019,” United States Centers for Disease Control and Prevention Morbidity and Mortality Weekly Report—Surveillance Summaries 70(9): 1-29; Kortsmit, K., Jatlaoui. T. C., Mandel, M. G., Reeves, J. A., Titilope, O., Petersen, E. and Whiteman, M. K. 2020. “Abortion Surveillance—United States, 2018,” United States Centers for Disease Control and Prevention Morbidity and Mortality Weekly Report—Surveillance Summaries 69(7): 1-29 with correction 70(49): 1717).

With respect to grouped vessel inlet/outlet confluence conduits (combined vessel diversion chute-pass confluence conduits, shared diversion chute multiple vessel inlet and outlet confluence conduits), shown in FIGS. 15C and 15D and addressed below in section 2b(1)(b)(b14), entitled Confluence Conduits: Connection of One or More Bloodlines to Flow through a Common Channel to the Same or a Different Number of Bloodlines with Self-leveling Apportionment of Outflow, in the subsection thereof entitled Tiny Vessel Grouped Inlet/Outlet Expandable Confluence Conduits. and in section 6, entitled Description of the Preferred Embodiments of the Invention, in the subsection thereof entitled Tiny Vessel Grouping Device, the early development of the immune system and the changes that occur in the maternal immune system during gestation suggest ways to facilitate the replacement of the heart or other organs in terms of practical methods and apparatus. Specifically, the immature condition of the fetal, and thereafter, the neonatal immune system and the impaired condition of the patient recommend the use of the sudden rather than the metered method for compound vascular bypass transplantation to reduce the operational duration

Tiny vessel grouping is an interim expedient devised to avert the need to apply a valve to each of the small vessels in a young pediatric patient. After an interval during which the baby is allowed to grow and gain in strength, the grouping devices are removed without an interruption in blood flow by bypass each vessel as depicted in FIG. 15D. When a larger autologous vessel segment is available, to anastomose the grouped small vessels to a single bypass is also an option but does not gain time. In FIGS. 15A, 15C, and 15D, the appearance of connection to the supraaortic arteries is meant as exemplary any other three or more arteries no less applicable.

Use of the sudden switch method does not discount the perioperative use of immune tolerance induction and rejection-counteractive medication, it does mean that the operation can be accomplished in less time and with less stress on the fetus. Favoring early replacement of a hopelessly malformed heart, the innate immune tolerance of the prenate develops at about two to three months into gestation, before which the antibodies of the gravida provided through the placenta and lactation serve to protect the baby (see, for example, Foessleitner, P., Pjevac, P., Granser, S., Wisgrill, L., Pummer L., and 5 others 2024. “The Maternal Microbiome in Pregnancy, Delivery, and Early-stage Development of Neonatal Microbiome after Cesarean Section: A Prospective Longitudinal Study,” Acta Obstetricia et Gynecologica Scandinavica (Malden, Massachusetts) 103(5): 832-841; Jiang, H. and Zhang, Q. 2024. “Gut Microbiota Influences the Efficiency of Immune Checkpoint Inhibitors by Modulating the Immune System (Review),” Online, Oncology Letters (Athens, Greece) 27(2): 87; Gao, C., Chen, Q., Hao, X., and Wang, Q. 2023. “Immunomodulation of Antibody Glycosylation through the Placental Transfer,” Online, MDPI [Multidisciplinary Digital Publishing Institute] International Journal of Molecular Sciences (Basel, Switzerland) 24(23): 16772; Jardine, L., Schim van der Loeff, I., Haq, I. J., and Sproat. T. D, R. 2023. “Gestational Development of the Human Immune System,” Immunology and Allergy Clinics of North America (Philadelphia, Pennsylvania) 43(1): 1-15: Xu. S., Zhang, G., Zhang, J., Liu, W., Wang. Y., and Fu. X. 2023. “Advances in Brain Tumor Therapy Based on the Magnetic Nanoparticles,” Online, International Journal of Nanomedicine (Auckland, New Zealand) 18:7803-7823; Feyaerts, D., Urbschat, C., Gaudilliere, B., and Stelzer, I. A. 2022. “Establishment of Tissue-resident Immune Populations in the Fetus, Online, Seminars in Immunopathology (Berlin, Germany) 44(6): 747-766; Hossain, Z., Reza, A. H. M. M., Qasem, W. A., Friel, J. K., and Omri, A. 2022. “Development of the Immune System in the Human Embryo,” Online, Pediatric Research (New York, New York) 92(4): 951-955; Mahanani, M. R., Abderbwih, E., Wendt, A. S., Deckert, A., Antia, K., and 4 others 2021. “Long-term Outcomes of in Utero Ramadan Exposure: A Systematic Literature Review,” Online, MDPI [Multidisciplinary Digital Publishing Institute] Nutrients (Basel, Switzerland) 13(12): 4511; Zhuang, B., Shang, J., and Yao, Y. 2021. “HLA-G: An Important Mediator of Maternal-Fetal Immune Tolerance,” Online, Frontiers in Immunology (Lausanne, Switzerland) 12:744324; Abu-Raya, B., Michalski, C., Sadarangani, M., and Lavoie, P. M. 2020. “Maternal Immunological Adaptation during Normal Pregnancy,” Online, Frontiers in Immunology (Lausanne, Switzerland) 11:575197; Apostol, A. C., Jensen, K, D. C., and Beaudin, A, E. 2020. “Training the Fetal Immune System through Maternal Inflammation-A Layered Hygiene Hypothesis,” Online, Frontiers in Immunology (Lausanne, Switzerland) 11:123; Jain, N. 2020. “The Early Life Education of the Immune System: Moms, Microbes, and (Missed) Opportunities,” Online, Gut Microbes (Philadelphia, Pennsylvania) 12(1): 1824564; Park, J.-E., Jardine, L., Gottgens, B., Teichmann, S. A., and Haniffa, M. 2020. “Prenatal Development of Human Immunity,” Science (Washington, D.C.) 368(6491): 600-603; Mimoun, A., Delignat. S., Peyron, I., Daventure, V., Lecerf. M., and 4 others 2020. “Relevance of the Materno-Fetal Interface for the Induction of Antigen-specific Immune Tolerance,” Online, Frontiers in Immunology (Lausanne, Switzerland) 11:810; Xu, X., Zhou, Y., and Wei, H. 2020. “Roles of HLA-G in the Maternal-Fetal Immune Microenvironment,” Online, Frontiers in Immunology (Lausanne, Switzerland) 11:592010; Guo, Y. L. 2019. “The Underdeveloped Innate Immunity in Embryonic Stem Cells: The Molecular Basis and Biological Perspectives from Early Embryogenesis,” Online, American Journal of Reproductive Immunology (New York, New York: 1989) 81(2): e13089 Hocevar, K., Maver, A., Vidmar Šimic, M., Hodžić, A., Haslberger, A., Premru Sersen, T., and Peterlin, B. 2019. “Vaginal Microbiome Signature is Associated with Spontaneous Preterm Delivery,” Online, Frontiers in Medicine (Lausanne, Switzerland) 10:6:201; Kiskova, T., Mytsko, Y., Schepelmann, M., Helmer, H., Fuchs, R., and 3 others 2019. “Expression of the Neonatal Fc [immune protein]-receptor in Placental-Fetal Endothelium and in Cells of the Placental Immune System,” Placenta (Amsterdam, North Holland, Netherlands) 78:36-43; Li, N., van Unen, V., Guo, N., Abdelaal. T., Somarakis, A., and 5 others 2019. “Early-life Compartmentalization of Immune Cells in Human Fetal Tissues Revealed by High-dimensional Mass Cytometry,” Online, Frontiers in Immunology (Lausanne, Switzerland) 14:10:1932; Stinson, L. F., Boyce, M. C., Payne, M. S., and Keelan, J, A. 2019. “The Not-so-sterile Womb: Evidence that the Human Fetus is Exposed to Bacteria Prior to Birth,” Online, Frontiers in Microbiology (Lausanne, Switzerland) 10:1124; Chougnet, C, A. 2018. “Human Fetal Immune Cells Fight Back,” Online, Science Translational Medicine 10(438): eaat3910; Jennewein, M. F., Abu-Raya, B., Jiang, Y., Alter, G., and Marchant, A. 2017. Transfer of Maternal Immunity and Programming of the Newborn Immune System,” Seminars in Immunopathology (Berlin, Germany) 39(6): 605-613; Ledford, H. 2017. “Fetal Immune System Revs Up in the Womb,” Nature volume 546, at https://www.nature.com; Marques, A. H., O'Connor, T. G., Roth, C., Susser, E., and Bjorke-Monsen, A. L. 2013. “The Influence of Maternal Prenatal and Early Childhood Nutrition and Maternal Prenatal Stress on Offspring Immune System Development and Neurodevelopmental Disorders,” Online, Frontiers in Neuroscience (Lausanne, Switzerland) 31:7:120; Mor, G. and Cardenas, I. 2010. “The Immune System in Pregnancy: A Unique Complexity,” Online, American Journal of Reproductive Immunology (New York, New York: 1989) 63(6): 425-433; Szekeres-Bartho, J. 2002. “Immunological Relationship between the Mother and the Fetus,” International Reviews of Immunology (London, England) 21(6): 471-495; Blaschitz, A., Hutter, H., and Dohr, G. 2001. “HLA [human leukocyte antigen] Class I Protein Expression in the Human Placenta,” Early Pregnancy (Cherry Hill, New Jersey). 5(1): 67-69; Sargent. I. L. 1993. “Maternal and Fetal Immune Responses during Pregnancy,” Experimental and Clinical Immunogenetics (Basel, Switzerland) 10(2): 85-102; Billington, W, D. 1992. “The Normal Fetomateimal Immune Relationship,” Bailliere's Clinical Obstetrics and Gynaecology (London, England) 6(3): 417-438; Hayward, A, R. 1983. “The Human Fetus and Newborn: Development of the Immune Response,” [March of Dimes] Birth Defects Original Article Series (White Plains, New York) 19(3): 289-294). In contrast to the innate immune system, the adaptive system will continue to develop until about the age of ten years old.

Both because the immune system in the prenate is immature and the duration of a metered transplant can take much more time imposing stress on the fetus, barring exceptional circumstances, use of the sudden switch method is indicated. The common (shared, collective) diversion chute-entered confluence conduit specially devised for use in prenates and neonates shown in FIGS. 15C and 15D and described to materially expedite the connection of vessels to the conduit through eliminating the need to separately connect each, the need to produce vascular valves and servovalves tiny enough to position along the tiny vessels of the patient calling for inordinate time and expense.

The use of grouped vessel inlet/outlet confluence conduits means that the advent of prenatal compound vascular heart and other solid organ transplantation need not await the production of minute vascular valves and servovalves. Such operations demand operators skilled in microsurgery, which is another reason to provide deceased donor sustainment centers likewise addressed above in section 2a in the subsection of like title. Fetal organ transplantation is an exception to the reduction in complexity that would allow more readily accessible general surgeons to perform such operations.

For use in late-term prenates and neonates, common input and output confluence conduits eliminate the need to produce and apply plunger solenoid-driven valves and servovalves in extremely small sizes at considerable expense each of which must be connected separately to a vessel. Moreover, each such connection must take into account level rotational angle, and the other means for ameliorating sidestem projection enumerated above in section 2a, Background of the Invention. General.

Use of grouped vessel inlet/outlet confluence conduits addressed in the sections specified shortly above and shown in FIGS. 15C and 15D to perform a compound vascular bypass heart transplant eliminates the need for costly micromachined valves and servovalves which to apply to each of the tiny vessels of the donor and recipient would demand inordinate microsurgical skill and operational duration stressful for the already impaired patient and exceed the skill of a readily accessible general surgeon. Placement of a grouped inlet/outlet confluence conduit is still a microsurgical procedure but one much reduced in the steps required compared to the need to separately place the valve for each inlet and outlet bloodline separately (see, for example, Papavasiliou, T., Ubong, S., Khajuria, A., Chazimichail, S., and Chan, J, C. Y. 2021. “3D Printed Chest Wall: A Tool for Advanced Microsurgical Training Simulating Depth and Limited View,” Plastic and Reconstructive Surgery Global Open 9(9): e3817; Kania, K., Chang, D. K., Abu-Ghname, A., Reece, E. M., Chu, C. K., and 3 others 2020. “Microsurgery Training in Plastic Surgery,” Plastic and Reconstructive Surgery Global Open 8(7): e2898; Abi-Rafeh, J., Zammit, D., Mojtahed, J. M., Al-Halabi, B. and Thibaudeau, S. 2019. “Nonbiological Microsurgery Simulators in Plastic Surgery Training: A Systematic Review,” Plastic and Reconstructive Surgery 144(3): 396e-507e; Javid, P., Aydin, A., Mohanna, P. N., Dasgupta, P. and Ahmed, K. 2019. “Current Status of Simulation and Training Models in Microsurgery: A Systematic Review,” Microsurgery 39(7): 655-668; Patel, E. A., Aydin, A., Desai, A., Dasgupta. P., and Ahmed, K. 2019. “Current Status of Simulation- based Training in Pediatric Surgery: Systematic Review,” Journal of Pediatric Surgery 54(9): 1884-1893; Gunasagaran, J., Rasid R. J., Mappiare, S. Devarajooh, C., and Ahmad, T. S. 2018. “Microgrids: A Model for Basic Microsurgery Skills Training,” Online, Malaysian Orthopedic Journal 12(2): 37-41).

With donors and recipients too young to have mature immunological function, whether the immunological conditions posed by the gravidae, and in some cases a potentially relevant paternal component, warrant metered rather than sudden switch compound vascular bypass heart transplantation as an enhancement to immune tolerance induction must be determined on the basis of genetic, or immunological diagnostics. The organ transfer is performed with the donor and recipient positioned side by side.

To simplify the surgical field not just visually, but to allow selected combinations of vessels to be medicated alike, their heart vessels are connected to one another through long-to-short switchable line confluence conduits, individual confluence conduits described in section 2b(1)(b)(b14), entitled Confluence Conduits: Connection of One or More Bloodlines to Flow through a Common Channel to the Same or a Different Number of Bloodlines with Self-leveling Apportionment of Outflow, as shown in FIG. 35A and those switchable in section 2b(1)(b)(b15), entitled Switchable Lines and Confluence Conduits: Long when Intercorporeal, to Short when Intracorporeal, Both Affording Connection of One or More Bloodlines to the Same or a Different Number of Bloodlines with Self-leveling Apportionment of Outflow, and shown schematically in FIGS. 35B thru 35F.

The line switchable confluence conduits are equipped with inline, or endoluminal, pumps, shown as part number 167 in FIG. 35B, to impart sufficient pressure as necessary to traverse the distance of the lines connecting the patients. As pictured in FIGS. 35C thru 35F, the long lines are switched to short lines before closing, and the anastomoses can be bypassed to switch flow between the organic and bypass routes to facilitate diagnostics and therapy. In FIG. 35B, entry 197 and exit 197′ bloodlines are matched in gauge to the vessels each connects and the confluence chamber would ordinarily have a volumetric flow-through rate in milliliters per second is equal to the sum of the volume delivery rates of the bloodlines connected to it.

Even in a late-term fetus, if separately valved, the tiny vessels would be costly and require much more time to connect by highly skilled operators, and that with the aid of binocular microscopes or robotic access. FIGS. 15C and 15D show a grouped entry confluence conduit devised for use in prenates, neonates, and toddlers to materially expedite the connection of vessels to the conduit by eliminating the need to separately valve each. Instead of connecting each vessel separately, the vessels entering are bunched together and made to pass a common, or shared, diversion chute controlled by a bistable linear plunger solenoid in a sudden switch or a servomotor in a metered switch transplant.

Hypothetically, merging the flow of multiple vessels and passing these through a common, or shared, confluence conduit with a singular inlet diversion chute, it is possible to provide a hybrid confluence conduit as a tuned shunt, a tuned shunt for a single input and output vessel shown in FIG. 22F. Use thus may prove advantageous in sudden switch compound vascular bypass organ transplantation where all flow is either diverted or not with solenoid-driven diversion chutes, but when the diversion chute drivers are servomotors, such control is inherently subsumed eliminating the need for an additional means of control.

A compound vascular bypass transplantation of the heart too early in development—well past the six weeks where a protobeat flutter can be detected—would never and need not be considered, because the heart and the other organs have not even fully formed with differentiated vasculature and the instatement of physiological function that might be measured to ascertain the normalcy of its function, which occurs well after the mere initiation of the pulse. While the fetus is primarily dependent upon its placental blood supply and drainage, the mere facts that the heart has initiated pulsatile circulation at about six weeks and that delivery sees the neonate having survived indicate that such function if not is essential to normal development.

Accordingly, the time to accomplish a diagnosis in order to determine the need for a replacement heart is properly deferred until the heart in development would exhibit distinct vasculature to allow a vessel-to-vessel connection and defects in performance that would severely impair the normal development of the fetus. When the embryo transitions to a fetus at about eight weeks, the circulatory system in development is too amorphous to be operated upon. Moreover, to justify consideration of a heart transplant, any aberrant performance detected must not be successfully treatable using conventional drugs, implantable pacemaker/cardioverter-defibrillators, or inadequate methods of surgical repair, such as a Norwood procedure.

If and only if these conditions are met, the transplant should be performed later in gestation when the vessels and conduction system have developed and the heartbeat is not the initial flutter generated by the anlage of the sinoatrial node but rather structured as in an adult and can be evaluated with the aid of echocardiography, or ultrasonography, and a cardiotocograph. While the fetal circulation is functionally distinct from the postnatal circulation, (see, for example, Finnemore, A. and Groves, A. 2015. “Physiology of the Fetal and Transitional Circulation,” Seminars in Fetal and Neonata Medicine 20(4): 210-216), this time is about 11 weeks into gestation, or just after the heart has reached its full development to six months (see, for example, Tan, C. M. J. and Lewandowski, A. J. 2020. “The Transitional Heart: From Early Embryonic and Fetal Development to Neonatal Life,” Fetal Diagnosis and Therapy 47(5): 373-386).

The transplant is administered by a disorder response system, which unlike that in the transplantation of a decompensated or failing heart in an adult where the system has been implanted—is consigned to an external console pending growth sufficient to allow such an ambulatory fully implanted support sytem to be implanted. When the patient is supported with such a system, the usual ‘mantra’ to discourage transplantation—that one has merely replaced one disease for another—carries no weight whatever. Unless the gravida faces imminent death, placental/umbilical connection to switch a fetus from her to another woman is discounted as posing needless risks. The immunological challenges in fetal transplantation should include evaluations of all four parents.

While the later fetus can be removed for open surgical repair with the umbilical cord remaining attached or the operation performed through a hysterotomy—placing the mother at risk and requiring that any following deliveries be cesarean—its heart so small that to accomplish a good surgical repair is difficult and not likely to provide more than a means for bridging to a transplant, preferably accomplished through metered switch, or bypass, transplantation. As the fetus develops, the solid organs and their blood vessels gradually emerge to become more and more distinct from the adjoining tissues. During this stage of less resolution, it is more productive and less traumatizing to replace rather than attempt to repair a severely malformed heart.

Where a placental transplant of an intact fetus would not, the instatement of normal perfusion through a heart transplant not only protects the development of the brain as well as all other tissues and organs but preserves the individuality of the fetus and its direct relation to the parents. The passive blood supply and drainage through the umbilical cord is adequate to support continued development of the fetus as a whole.

Once the fetus becomes dependent upon its own developing heart at about six weeks, any secondary malformity that resulted from deviant circulation will not spontaneously disappear, although replacement with a good heart will remove the cause and the damage will probably be surgically correctible. Otherwise, irreversible and left untreated, the defect will have been locked in as the base upon which continued development must build, a defect no more than adapted to. Surgical repair or transplantation of the heart or other solid organ can be performed only once the embryo has progressed to a later stage in fetal morphogenesis at which the organ and the defect to be addressed have become distinct and sufficiently solidified to allow cautious manipulation. For the major vessels, this is at about the tenth week in gestation.

Intervention to interrupt the continued development of a severe heart malformity in utero is currently limited to transcatheteric or transluminal techniques such as transcatheter septostomy, valvuloplasty, and the widening of constricted passageways, which often succeeds in lessening the severity of what otherwise would have become a devastating defect, but given that even open surgical repair cannot achieve normal function, such measures are incapable of providing a cure (see, for example, the references cited above in section 2. Background of the Invention. a. General). Before the stage in prenatal development when the heart and its vessels emerge as distinct, a fetus with an unsurvivable or severely debilitating defect can be replaced by umbilical switch fetal transplantation as addressed below in the section of like title.

Primarily because it is extracardiac and completely avoids incision into or manipulation of the tiny anatomy within the heart, but also because it can omit machine support, cardioplegia, cross-clamping, and the need for general rather than regional anesthesia for both prenate and mother, switch heart transplantation affords a method for replacing a severely defective heart in development with a good heart and for doing so with relatively little dissection. Even when metered, matching of both mothers and their fetuses must be thorough, with immune tolerance induction initiated prior to the time for onset of the tolerance induction agents used.

A sufficiency of replacement hearts notwithstanding, the heart placed may often be taken from an older infant as an expedient, and while to some extent oversized for the recipient, should adapt to the lesser load within a relatively short interval (see, for example, Shin, H. J., Jhang, W. K., Park, J. J., Yun, T. J., Kim, Y. H., and 3 others 2011. “Heart Transplantation in Pediatric Patients: Twelve-year Experience of the Asan Medical Center,” Journal of Korean Medical Science 26(5): 593-598; Kertesz, N. J., Gajarski, R. J., Towbin, J. A., and Geva. T. 1995. “Effect of Donor-recipient Size Mismatch on Left Ventricular Remodeling after Pediatric Orthotopic Heart Transplantation,” American Journal of Cardiology 76(16): 1167-1172).

A relatively simple implanted automatic response system to detect and directly deliver an immunosuppressant, antimicrobials, and anti-inflammatoires is placed, and the prescription-program of the microcontroller used controls not only this postoperative maintenance function but the redirection of the recipient's circulation from the native to the donor heart. During the ensuing week or so, the automatic maintenance system releases antihypertensive medication to avert the seizures and coma that otherwise tend to follow the placement of an oversized heart (see, for example, Reichart, B. 1992, below under section 2b(1)(b)(b3), entitled Neonatal and Infant Compound Vascular Bypass, or Sudden Switched-organ Transplantation of the Heart. Since ordinarily, the problem of immune rejection between two immunologically immature subjects is minimal, the procedure is performed using the sudden switch technique with solenoid rather than servo valve-jackets.

Initially, the surgical repair of congenital heart defects could no more than prevent the imminent death of the child so affected. In the face of this urgency, and unable to predict the outcome of early procedures, means for improving longevity and normal development remained unpredictable and had to be relegated to future experience. Eventually, it became clear that while relatively minor defects such as an aperture in the interatrial septum once closed would cease to be a problem, more serious malformities such as a univentricular heart could not be repaired to the degree that nonpulsatile passive blood flow would be avoided to allow normal development. Often, children bom with a severely malformed heart lived eleven or so years after surgery in poor health and with impaired development. Even today, severe defects continue to elude repair that achieves normal circulation, leaving a heart transplant as the only remaining option.

A heart so mal formed that it cannot be sufficiently repaired to allow a normal life is best removed and replaced with a normal heart. However, at the inception of intracardiac heart surgery in 1952, the prospect of heart transplantation, not begun until 15 years later, was no more than dimly foreseen, and it would not be until the mid-1990s that immunosuppressants would allow survival of 20 or more years following the operation (see, for example, John, M. and Bailey, L. L. 2018. Op cit. in section 2a, entitled Background of the Invention. General; Mastrobuoni, S., Dell'Aquila, A. M., Azcarate, P. M., Rabago. G., and Herreros, J. 2012. “Long-term Survival (>20 Years) following Heart Transplantation,” Journal of Cardiovascular Surgery (Turin, Italy) 53(5): 677-684).

The advent of immunosuppressant drugs, cyclosporine in particular, considerably reduced the incidence of rejection. However, immunosuppressants do not provide long-term freedom from adverse graft sequelae (see, for example, Kaundal. U., Bagai, U., and Rakha, A. 2018, Op cit., above in section 2a, entitled Background of the Invention. General). Thereafter, it was realized that preliminary immune tolerance could be increased with a reduction in long-term complications by exposing the recipient to cells taken from the donor and introduced into the recipient in advance of the transplant. The benefits gained by allowing a reduction in the dosing of immunosuppressives and their side effects as made possible by compound vascular bypass transplantation are reviewed above in section 2a, Background. General.

Anything that will reduce the need for immunosuppressants to include cyclosporine, such as the immune tolerance induction and associated degree of acclimation accomplished during metered compound vascular bypass solid organ transplantation will reduce the risk of these side effects. The metered technique accomplishes this through a degree of microchimerism and acclimation accomplished during extended reciprocal donor-recipient cross-circulation which is the process by which the graft organ is gradually transferred from the circulatory system of the donor into that of the recipient. This process should reduce but not completely eliminate the need for immunosuppressives. Adverse side effects associated with cyclosporine include lymphoma, other malignancies, hepatotoxicity, nephrotoxicity, hypertension, cephalalgia, and the risk of infection.

Still other complications of cyclosporine include paresthesias, hyperlipidemia, dyspnea, convulsions, pancreatitis, elevated blood pressure, fever, diarrhea, emesis, pruritis, and still others, making steroids worth consideration (see, for example, Tapia. C., Nessel. T. A., and Zito, P. M. 2021. Cyclosporine, Online, Treasure Island, Florida: StatPearls Publishing Co.; Teimouri. A., Ahmadi, S. R., Ardakani, S, A. and Foroughian, M. 2020. “Cyclosporine A-based Immunosuppressive Therapy-induced Neurotoxicity: A Case Report,” Open Access Emergency Medicine 12:93-97; Lidicoat. A. M. and Lavelle, E, C. 2019. “Modulation of Innate Immunity by Cyclosporine A,” Biochemical Pharmacology 163:472-480; Panettieri, R. A., Schaafsma, D., Amrani, Y., Koziol-White, Ostrom, R., and Tliba, O. 2019. “Non-genomic Effects of Glucocorticoids: An Updated View,” Trends in Pharmacological Sciences 40(1): 38-49; Ponticelli, C. and Glasscock, R. J. 2019. “Prevention of Complications from Use of Conventional Immunosuppressants: A Critical Review,” Journal of Nephrology 32(6): 851-870; Shin, H. S., Grgic, I., and Chandraker, A. 2019. Novel Targets of Immunosuppression in Transplantation,” Cinics in Laboratory Medicine 39(1): 157-169; Tedesco, D. and Haragsim, L. 2012. “Cyclosporine: A Review,” Journal of Transplantation 2012:230386).

Other immunosuppressives likewise pose problems. Posttransplantation complications are “usually related to surgical procedure of transplantation, primary disorders causing failure of transplanted organ, opportunistic infections, and neurotoxicity of immunosuppressive medications” (Zivcovic, S, A. and Abdel-Hamid, H. 2010. “Neurologic Manifestations of Transplant Complications,” Neurologic Clinics 28(1): 235-251)—all factors reduced if not eliminated in metered compound vascular bypass transplantation (see also Puckett. Y., Gabbar, A., and Bokhari, A, A. 2022. “Prednisone”, Online, Treasure Island, Florida: StatPearls Publishing Co.; Yasir, M., Goyal, A. and Sonthalia, S. 2021. “Corticosteroid Adverse Effects,” Online, Treasure Island, Florida: StatPearls Publishing Co.; Benvenuto, L. J., Anderson, M. R., and Arcasoy, S. M. 2018. “New Frontiers in Immunosuppression,” Journal of Thoracic Disease 10(5): 3141-3155; Anghel. D., Tanasescu. R., Campeanu, A. Lupescu, I. Podda, G. and Bajenaru, O. 2013. “Neurotoxicity of Immunosuppressive Therapies in Organ Transplantation,” Maedica (Bucharest, Romania) 8(2): 170-175; Sklar, E. M. 2006. “Post-transplant Neurotoxicity: What Role do Calineurin Inhibitors, Actually Play?,” American Journal of Neuroradiology 27(8): 1602-1603).

Necessarily but indiscriminately impairing immune responsiveness, the essential drawbacks of immunosuppression maintenance drugs cannot have been ameliorated in their latest incarnations, which may account for the virtual cessation in the search for newer drugs of this kind for almost three decades. All drugs pose the risk of adverse side effects, and immunosuppressives severe side effects. The first maintenance immunosuppressive, sirolimus, or rapamycin, was discovered in 1975, cyclosporine, discovered in 1971, did not enter use until 1983, tacrolimus in 1987, mycophenolate mofetil in 1995, immunosuppression-allied anti-inflammatory glucocorticoids discovered in the 1940s were not used for immunosuppression until around 1995, and everolimus entered use in 2009.

Immunosuppressives used in organ transplantation are usually combined, but this does relatively little to reduce their side effects. Because immunosuppressive drugs cannot be reformulated to overcome their systemically indiscriminate action and side effects, alternative means are needed to effectuate immune tolerance induction, allow the doses used to be reduced, and target their action. The copending applications and patents specified above respond to these objectives. Metered compound vascular bypass solid organ transplantation in particular effectuates a measure of immune tolerance induction inherently in the donor-recipient reciprocal cross-circulation used to transfer the graft organ from the donor to the recipient.

For use in children, dosing if miscalculated can exert adverse effects on growth and development (see, for example, Tonshoff, B. 2020. “Immunosuppressants in Organ Transplantation,” in Handbook of Experimental Pharmacology, New York, New York: Springer Nature, volume 261, pages 441-469; Zhang-Velten, E. R., Gerber, D. E., and Westover, K, D. 2020. “Thirteen-year Survival in a Patient with Diffuse Bilateral Lepidic-predominant Adenocarcinoma: A Case Report of Lung Transplantation and Local Salvage,” JTO [Journal of Thoracic Oncology] Clinical Research Reports 1(4): 100094; Wijesinha, M., Hirshon, J. M., Terrin, M., Magder, L., Brown, C. Stafford, K., and lacono, A. 2019. “Survival Associated with Sirolimus Plus Tacrolimus Maintenance without Induction Therapy Compared with Standard Immunosuppression after Lung Transplantation,” Journal of the American Medical Association Open Network 2(8): e1910297; Coutinho, A, E. and Chapman, K, E. 2011. “The Anti-inflammatory and Immunosuppressive Effects of Glucocorticoids, Recent Developments and Mechanistic Insights,” Molecular and Cellular Endocrinology 335(1): 2-13).

Reciprocal donor-recipient cross-microchimerization during extended reciprocal donor-recipient cross-circulatory transfusion reduces the shock presented by in a sudden confrontation of native with alien tissue and therefore the amount of immunosuppressants required (see, for example, Fan, Z., Deng. J., Li, P. Y., Chery, D. R., Su. Y., and 5 others 2019. “A New Class of Biological Materials: Cell Membrane-derived Hydrogel Scaffolds,” Biomaterials 197:244-254; Li, P. Y., Fan, Z., and Cheng, H. 2018. “Cell Membrane Bioconjugation and Membrane-derived Nanomaterials for Immunotherapy,” Bioconjugate Chemistry 29(3): 624-634; Li, J., Li, C., Zhuang, Q., Peng. B., Zhu, Y., Ye, Q., and Ming, Y. 2019. “The Evolving Roles of Macrophages in Organ Transplantation,” Journal of Immunology Research 2019:5763430; Samy, K. P. and Brennan, T. V. 2018. “Dendritic Cell Therapy in Transplantation, Phenotype Governs Destination and Function,” Transplantation 102(10): 1593-1594; Meng. X., Chen, M., Su. W., Tao, X., Sun, M., and 4 others 2018. “The Differentiation of Mesenchymal Stem Cells to Vascular Cells Regulated by the HMGB1 [high mobility group box 1]/RAGE [receptor for advanced glycation end-product] Axis: Its Application in Cell Therapy for Transplant Arteriosclerosis,” Stem Cell Research and Therapy 9(1): 85; Scalea. J. R., Tomita, Y., Lindholm, C. R., and Burlingham, W. 2016. “Transplantation Tolerance Induction: Cell Therapies and Their Mechanisms,” Online, Frontiers in Immunology (Lausanne, Switzerland) 7:87; Hivelin, M., Klimczak, A., Cwykiel, J., Sonmez, E., Nasir, S., Gatherwright, J., and Siemionow, M. 2016. “Immunomodulatory Effects of Different Cellular Therapies of Bone Marrow Origin on Chimerism Induction and Maintenance across MHC Barriers in a Face Allotransplantation Model,” Archivum Immunologiae et Therapiae Experimentalis (Warsaw, Poland) 64(4): 299-310; Hock, K., Mahr. B., Schwarz, C., and Wekerle, T. 2015. “Deletional and Regulatory Mechanisms Coalesce to Drive Transplantation Tolerance through Mixed Chimerism,” European Journal of Immunology 45(9): 2470-2479; Im, K. I., Park, M. J., Kim, N., Lim, J. Y., Park, H. S., and 6 others 2014. “Induction of Mixed Chimerism Using Combinatory Cell-based Immune Modulation with Mesenchymal Stem Cells and Regulatory T Cells for Solid-organ Transplant Tolerance,” Stem Cells and Development 23(19): 2364-2376; Pilat, N., Hock, K., and Wekerle, T. 2012. Op cit., section 2a; additional references cited in section 2a and in sections to follow).

Open fetal surgery whereby the prenate is removed by cesarean delivery without disconnection of the umbilical cord and returned to the uterus upon completion, and percutaneous fetoscopic surgery through the abdominal wall and uterus, have been developed for over a decade. That this approach is limited to noncardiac defects such as spina bifida and twin-to-twin transfusion syndrome is mistaken; however, fetal heart transplantation yet to be developed, it is expected that the relatively trauma and ischemia-free advantages in compound vascular bypass, or sudden switched-organ, heart transplantation will have a role in making this advancement possible. In contrast, neonatal heart transplantation has been possible for over 40 years; however, by then, the secondary consequences for prenatal development dependent upon the normal functioning of the heart may have already transpired. Neonatal heart transplantation is address in the section to follow.

To interdict the emergence of hypoplastic left heart syndrome and univentricular malformity, ultrasound-guided transcatheteric treatment has proven effective and safer than incisional surgery (see, for example, Tulzer, A., Arzt. W., Gitter, R., Prandstetter, C., Grohmann, E., Mair, R., and Tulzer, G. 2018. “Immediate Effects and Outcome of In-utero Pulmonary Valvuloplasty in Fetuses with Pulmonary Atresia with Intact Ventricular Septum or Critical Pulmonary Stenosis,” Ultrasound in Obstetrics and Gynecology 52(2): 230-237; Schidlow, D. N., Freud, L., Friedman, K., and Tworetzky, W. 2017. “Fetal Interventions for Structural Heart Disease,” Echocardiography 34(12): 1834-1841; Gellis, L. and Tworetzky, W. 2017. “The Boundaries of Fetal Cardiac Intervention: Expand or Tighten?,” Seminars in Fetal and Neonatal Medicine August 31. pii: S1744-165X (17) 30095-1; Maselli, K. M. and Badillo, A. 2016. “Advances in Fetal Surgery,” Annals of Translational Medicine 4(20): 394; Lapa Pedreira, D, A. 2016. “Advances in Fetal Surgery,” Einstein (Sao Paulo, Brazil) 14(1): 110-112; Freud, L, R. and Tworetzky, W. 2016. “Fetal Interventions for Congenital Heart Disease,” Current Opinion in Pediatrics 28(2): 156-162; Yuan, S. M. and Humuruola, G. 2016. “Fetal Cardiac Interventions: Clinical and Experimental Research,” Postępy w Kardiologii Interwencyjnej [Advances in Interventional Cardiology] 12(2): 99-107; Matte, G. S., Connor, K. R., Toutenel, N. A., Gottlieb, D., and Fynn-Thompson, F. 2016. “A Modified EXIT [ex utero intrapartum treatment]-to-ECMO [extracorporeal membrane oxygenation] with Optional Reservoir Circuit for Use during an EXIT [ex utero intrapartum treatment] Procedure Requiring Thoracic Surgery,” Journal of Extracorporeal Technology 48(1): 35-38; Kulkami, M., Gokulakrishnan, G., Price, J., Fernandes, C. J., Leeflang, M., and Pammi, M. 2015. “Diagnosing Significant PDA [patent ductus arteriosus] Using Natriuretic Peptides in Preterm Neonates: A Systematic Review,” Pediatrics 135(2): e510-e525; Pratt. R., Deprest. J., Vercauteren. T., Ourselin, S., and David, A. L. 2015. “Computer-assisted Surgical Planning and Intraoperative Guidance in Fetal Surgery: A Systematic Review,” Prenatal Diagnosis 35(12): 1159-1166; Sala, P., Prefumo, F., Pastorino, D., Buffi. D., Gaggero, C. R., Foppiano, M., De Biasio, P. 2014. “Fetal Surgery: An Overview,” Obstetrical and Gynecological Survey 69(4): 218-228; Yuan, S. M. 2014. “Fetal Cardiac Interventions: An Update of Therapeutic Options,” Revista Brasileira de Cirurgia Cardiovascular [Brazilian Journal of Cardiovascular Surgery] 29(3): 388-395; Merz, W. M., Leufgen, C., Fimmers. R., Stoffel- Wagner. B., and Gembruch, U. 2014. “Reference Intervals for N-terminal Pro-B-type Natriuretic Peptide in Amniotic Fluid Between 10 and 34 Weeks of Gestation,” Online, PLoS [Public Library of Science] One 9(12): e114416; Chauhan, S. P., Blackwell, S. B., and Ananth, C. V. 2014. “Neonatal Brachial Plexus Palsy: Incidence, Prevalence, and Temporal Trends,” Seminars in Perinatology 38(4): 210-218; Adzick, N. S. 2013, Op. cit., above in this section; Taghavi, K. and Beasley, S. 2013. “The Ex Utero Intrapartum Treatment (EXIT) Procedure: Application of a New Therapeutic Paradigm,” Journal of Paediatrics and Child Health 49(9): E420-E427; Diemert, A., Diehl, W., Glosemeyer, P., Deprest. J., and Hecher, K. 2012. “Intrauterine Surgery—Choices and Limitations,” Deutsches Ārzteblatt [German Medical Journal] International 109(38): 603-638; Partridge, E, A. and Flake, A. W. 2012. “Maternal-fetal Surgery for Structural Malformations,” Best Practice and Research, Clinical Obstetrics and Gynaecology 26(5): 669-682; Artz, T. and Tulzer, G. 2011. “Fetal Surgery for Cardiac Lesions,” Prenatal Diagnosis(Chichester, Sussex, England) 31(7): 695-698; Hartmann, K. E., McPheeters, M, L., Chescheir, N. C., Gillam-Krakauer, M., McKoy, J. N., and 4 others 2011. “Evidence to Inform Decisions about Maternal-fetal Surgery: Technical Brief,” Obstetrics and Gynecology 117(5): 1191-1204; Abraham, R. J., Sau, A., and Maxwell, D. 2010. “A Review of the EXIT (Ex utero Intrapartum Treatment) Procedure,” Journal of Obstetrics and Gynaecology 30(1): 1-5; Kunisaki, S. M. and Jennings, R. W. 2008. “Fetal Surgery,” Journal of Intensive Care Medicine 23(1): 33-51; Ange. M., Peiry. B., and Pfister, R, E. 2007. “Ex Utero Intrapartum Treatment (EXIT), a Resuscitation Option for Intra-thoracic Foetal Pathologies,” Swiss Medical Weekly 137(19-20): 279-285; Hankins, G. D., Clark, S. M., and Munn, M, B. 2006. “Cesarean Section on Request at 39 weeks: Impact on Shoulder Dystocia, Fetal Trauma, Neonatal Encephalopathy, and Intrauterine Fetal Demise,” Seminars in Perinatology 30(5): 276-287; Hirose, S. and Harrison, M, R. 2003. “The Ex Utero Intrapartum Treatment (EXIT) Procedure,” Seminars in Neonatology 8(3): 207-214; Hanley, F. L. 1994. “Advances in Cardiac Surgery Europe Pubmed Central 5:47-74).

Percutaneous, that is, transcatheteric or transluminal, valvuloplasty haa been applied to prenates and neonates (see, for example, Yu, J. J., Pan, W., Zhang, Z. W., Zhou, C. B., Pang, C. C., and 3 others 2018. “Intrauterine Cardiac Intervention for Fetal Pulmonary Valve Obstruction Lesion,” Chinese Medical Journal (English edition) 131(1): 103-104; Tulzer, A., Arzt. W., Gitter, R., Prandstetter, C., Grohmann, E., Mair, R., and Tulzer, G. 2018. “Immediate Effects and Outcome of In-utero Pulmonary Valvuloplasty in Fetuses with Pulmonary Atresia with Intact Ventricular Septum or Critical Pulmonary Stenosis,” Ultrasound in Obstetrics and Gynecology 52(2): 230-237; Edwards, A., Veldman, A., Nitsos, I., Chan, Y., Brew, N., and 4 others 2015. “A Percutaneous Fetal Cardiac Catheterization Technique for Pulmonary Valvuloplasty and Valvulotomy in a Mid-gestation Lamb Model,” Prenatal Diagnosis 35(1): 74-80).

Repairable by cardiac surgeons, septal defects are pertinent to transplantation only when accompanied by more extensive congenital malformities (see, for example, Dewan, S., Krishnamurthy, A., Kole, D., Conca, G., Kerckhoffs, R., and 5 others 2017. “Model of Human Fetal Growth in Hypoplastic Left Heart Syndrome: Reduced Ventricular Growth Due to Decreased Ventricular Filling and Altered Shape,” Online, Frontiers in Pediatrics 5:25; Kloesel, B., DiNardo, J. A., and Body, S. C. 2016. “Cardiac Embryology and Molecular Mechanisms of Congenital Heart Disease: A Primer for Anesthesiologists,” Anesthesia and Analgesia 123(3):551-569; Burns. T., Yang, Y., Hiriart, E., and Wessels A. 2016. “The Dorsal Mesenchymal Protrusion and the Pathogenesis of Atrioventricular Septal Defects,” Journal of Cardiovascular Development and Disease 3(4). pii: 29: Zhu, H. 2016. “Forkhead Box Transcription Factors in Embryonic Heart Development and Congenital Heart Disease,” Life Sciences 144:194-201; Briggs. L. E., Kafa, J., and Wessels, A. 2012. “The Pathogenesis of Atrial and Atrioventricular Septal Defects with Special Emphasis on the Role of the Dorsal Mesenchymal Protrusion,” Differentiation 84(1):117-130; Barron, D. J., Kilby, M. D., Davies, B., Wright, J. G., Jones, T. J., and Brawn, W. J. 2009. “Hypoplastic Left Heart Syndrome,” Lancet (London, England) 374(9689):551-564; Shinebourne, E. A., Babu-Narayan, S. V., and Carvalho, J. S. 2006. “Tetralogy of Fallot: From Fetus to Adult,” Heart 92(9):1353-1359; Sakabe, M., Matsui, H., Sakata, H., Ando, K., Yamagishi, T., and Nakajima, Y. 2005. “Understanding Heart Development and Congenital Heart Defects through Developmental Biology: A Segmental Approach,” Congenital Anomalies (Kyoto) 45(4):107-118; Homberger, L. K., Sanders, S. P., Sahn, D. J., Rice, M. J., Spevak, P. J., Benacerraf. B. R., McDonald, R. W., and Colan, S. D. 1995. “In Utero Pulmonary Artery and Aortic Growth and Potential for Progression of Pulmonary Outflow Tract Obstruction in Tetralogy of Fallot,” Journal of the American College of Cardiology 25(3):739-745; Goor, D. A., Dische, R., and Lillehei, W. 1972. “The Conotruncus. I. Its Normal Inversion and Conus Absorption,” Circulation 46(2):375-384; Goor, D. A. and Edwards, J. E. 1972. “The Conotruncus. II. Report of a Case Showing Persistent Aortic Conus and Lack of Inversion of the Truncus (a Bulboventricular Heart),” Circulation 46(2):385-389; additional citations below).

The presence of alloantigens in the bloodstream of the infant a determinant as to the choice of sudden or metered compound vascular bypass, or sudden switched-organ transplantation, on balance, breastfeeding is distinctly beneficial for an infant from an immunological as well as a nutritional standpoint (see, for example, Kelishadi. R., Hasanghaliaei, N., Poursafa, P., Keikha, M., Ghannadi. A., Yazdi, M., and Rahimi, E. 2016. “A Randomized Controlled Trial on the Effects of Jujube Fruit on the Concentrations of Some Toxic Trace Elements in Human Milk,” Journal of Research in Medical Sciences 21:108; Mead, M. N. 2008. “Contaminants in Human Milk: Weighing the Risks Against the Benefits of Breastfeeding,” Environmental Health Perspectives 116(10):A427-A434).

Barring unforeseen circumstances, such as parental chimerization during gestation to a degree considered significant or blood transfusions required by trauma before delivery, switch transplantation in a fetus or neonate as immunologically premature, is ordinarily with the sudden rather than the metered switch technique. After delivery, exposure to pathogens and exogenous matter such as tainted baby formula educe the development of responsive antigens which increase daily.

Alloantigens should exert little if any effect on the fetus itself whose immune system is undeveloped, but these may conflict with a donor organ taken from an older infant with a more developed immune system; however, metered compound vascular bypass, or sudden switched-organ transplantation much less preparatory apheresis should rarely if ever be necessary (see, for example, Losio, M. N., Pavoni, E., Finazzi, G., Agostoni, C., Daminelli, P. and 3 others 2018. “Preparation of Powdered Infant Formula: Could Product's Safety Be Improved?,” Journal of Pediatric Gastroenterology and Nutrition 67(4):543-546; Ojo-Okunola, A., Nicol, M.3, and du Toit, E. 2018. “Human Breast Milk Bacteriome in Health and Disease,” Nutrients 10(11). pii: E1643; Miklavcic, J. J., Badger, T. M., Bowlin, A. K., Matazel, K. S., Cleves, M. A., and 6 others 2018. “Human Breast-milk Feeding Enhances the Humoral and Cell-mediated Immune Response in Neonatal Piglets,” Online, Journal of Nutrition (Springfield, Illinois) 148(11):1860-1870; Yeruva, L., Spencer, N. E., Saraf. M. K., Hennings. L., Bowlin, A. K., and 7 others 2016. “Formula Diet Alters Small Intestine Morphology, Microbial Abundance and Reduces VE [vascular endothelial]-cadherin and IL [interleukin]-10 Expression in Neonatal Porcine Model,” Online, BioMed Central Gastroenterology 16:40; Parigi, S. M., Eldh, M., Larssen, P., Gabrielsson, S., and Villablanca, E. J. 2015. “Breast Milk and Solid Food Shaping Intestinal Immunity,” Online, Frontiers in Immunology (Lausanne, Switzerland) 6:415).

A fetus with cardiorenal syndrome boding significant heart and kidney impairment offers an example of the intricacy encountered with chimerization in preparation for multiple organ transplantation (see, for example, Leufgen, C., Gembruch, U., Stoffel-Wagner. B., Fimmers. R., and Merz, W. M. 2017. “N-terminal Pro-B-type Natriuretic Peptide in Amniotic Fluid of Fetuses with Known or Suspected Cardiac Load,” Online, PLoS [Public Library of Science] One 12(5):e0177253 (Op cit.); Carvajal, J. A., Ferrer, F. A., Araya, F. I., Delpiano, A. M. 2017. “Normal Amino- terminal Pro-brain Natriuretic Peptide (NT-proBNP) Values in Amniotic Fluid” Clinical Biochemistry 50(1-2):23-26 (Op cit.); Savla, J., Lin, K. Y., Pradhan, M., Ruebner, R. L., Rogers, R. S., and 6 others 2015. Op cit.; Merz, W. M., Kübler, K., Fimmers, R., Willruth, A., Stoffel-Wagner, B., and Gembruch, U. 2013. Op cit.). Severity to both heart and kidneys greater than can be satisfactorily repaired calls for replacement.

Accomplished by means of a sudden or metered switch procedure and depending upon the conditions of the donor, mother, and fetus, at least from a single donor, the three organs can be replaced, preferably, at the same time, or if necessary, sequentially. A fetus or neonate with intact heart and kidneys pending unavoidable miscarriage must be found and tissue components from the recipient administered to produce immune tolerance. Even if the heart and kidneys appear normal, a fetus presenting with severe genetic defects such as anencephaly or iniencephaly clausus or apertus should be suspected to harbor other genetic and pleiotropic defects and avoided. Before bringing donor and recipient together, the match is evaluated through standard cytotoxic-crossmatch assays and age notwithstanding, noninvasive gene expression profiling tests and if necessary, minimal tissue biopsies taken from both.

Such testing continues over the period of manipulative cross-chimerization and in the recipient, following transplantation (Pham, M. X., Teuteberg, J. J., Kfoury, A. G., Starling. R. C., Deng. M. C., and 11 others with 39 collaborators 2010. “Gene-expression Profiling for Rejection Surveillance after Cardiac Transplantation,” New England Journal of Medicine 362(20):1890-1900; Pham, M. X., Deng. M. C., Kfoury, A. G., Teuteberg, J. J., Starling, R. C., and Valantine, H. 2007. “Molecular Testing for Long-term Rejection Surveillance in Heart Transplant Recipients: Design of the Invasive Monitoring Attenuation through Gene Expression (IMAGE) Trial,” Journal of Heart and Lung Transplantation (New York, New York) 26(8):808-814).

That maternal-to-fetus cellular cross-microchimerism as seen in peripheral blood increases as gestation progresses, and that open fetal surgery increases maternal-to-fetus cellular cross-microchimerism suggests application to donor-recipient reciprocal cross circulatory microchimerism in organ transplantation, whether through infusion periprocedural to a sudden compound vascular bypass, or sudden switched-organ transplantation, or automated during a metered compound vascular bypass, or sudden switched-organ transplantation (Shree, R., Harrington, W. E., Kanaan, S. B., Forsyth, A., Cousin, E., Lopez, A., Nelson, J. L., and Gammill, H. S. 2019. “Fetal Microchimerism by Mode of Delivery: A Prospective Cohort Study,” British Journal of Obstetrics and Gynaecology 126(1):24-31; Chougnet, C. A. 2018. “Human Fetal Immune Cells Fight Back,” Science Translational Medicine 10(438). pii: eaat3910; Kinder, J. M., Stelzer, I. A., Arck, P. C., and Way, S. S. 2017. “Immunological Implications of Pregnancy-induced Microchimerism,” Nature Reviews. Immunology 17(8):483-494; Lunsford, K. E., Bodzin, A. S., Markovic, D., Zarrinpar, A., Kaldas, F. M., and 7 others 2017, Op cit. above under section 2b(1)(b), entitled Metered Compound Vascular Bypass, or Metered Switched-organ, Transplantation; Hansen, K. P. and Kamper-Jørgensen, M. 2017. “Microchimerism and Cancer in Women,” (in Danish with abstract at Pubmed), Ugeskrift for Laeger (Weekly Journal for Physicians/Journal of the Danish Medical Association) 179(9); Kinder, J. M., Jiang, T. T., Ertelt, J. M., Xin, L., Strong, B. S., Shaaban, A. F., and Way, S. S. 2015. “Cross-generational Reproductive Fitness Enforced by Microchimeric Maternal Cells,” Cell 162(3):505-515; Jeanty, C., Derderian, S. C., and Mackenzie, T. C. 2014. “Maternal-fetal Cellular Trafficking: Clinical Implications and Consequences,” Current Opinion in Pediatrics 26(3):377-382; Nijagal. A. and Mackenzie, T. C. 2013. “Clinical Implications of Maternal-fetal Cellular Trafficking,” Seminars in Pediatric Surgery 22(1):62-65; Saadai. P., Lee. T. H., Bautista, G., Gonzales, K. D., Nijagal, A., and 9 others 2012. “Alterations in Maternal-fetal Cellular Trafficking after Fetal Surgery,” Journal of Pediatric Surgery 47(6):1089-1094; Saadai. P. and Mackenzie, T. C. 2012. “Increased Maternal Microchimerism after Open Fetal Surgery,” Chimerism 3(3):1-3; Adams Waldorf, K. M., Gammill, H. S., Lucas, J., Aydelotte, T. M., Leisenring. W. M., Lambert, N. C., and Nelson, J. L. 2010. “Dynamic Changes in Fetal Microchimerism in Maternal Peripheral Blood Mononuclear Cells, CD4+ and CD8+ Cells in Normal Pregnancy,” Placenta 31(7):589-594; Adams Waldorf, K. M. and Nelson, J. L. 2008. “Autoimmune Disease during Pregnancy and the Microchimerism Legacy of Pregnancy,” Immunological Investigations 37(5):631-644).

Today, based upon the medical merits and prompt availability, only repair likely to result in sustained life without heart-related problems justifies repair rather than transplantation. A heart transplanted into a small child usually requires replacement in about eleven years, but critically, compared to an inadequate repair at birth, averts the maldevelopment that follows an inadequate repair, the constant health problems, and the severely degraded potential for accomplishment. Means for increasing organ availability—by developing treatments that allow less than ideal organs to be restored and used with success, for example, and by developing and optimizing drugs and combinations of drugs for sustaining organs once placed are being aggressively pursued throughout the developed world.

In this regard, no method for supporting a transplant, certainly none limited to a clinic or dependent upon regimen compliance, can exceed the immediacy and precision of an implanted automatic response system. The discomfort upon placement is eclipsed by that of the procedure itself, and with healing and time, the sensation of small implants with rounded corners and edges, cushioned where necessary, recede more and more into unconsciousness.

Following a program of mismatch prevention testing (cytotoxic-crossmatch assay, AlloMap Molecular Expression Testing, gene expression testing, and so on) and preliminary tolerance induction, such an implanted system, with power restoration by extracorporeal transcutaneous power transfer, can instantly detect signs, or biomarkers, of rejection whether hyperacute, acute, or chronic (see, for example, Agodi, A., Barchitta, M., Maugeri, A., Basile. G., Zamboni, M., and 3 others 2019. “Unveiling the Role of DNA Methylation in Kidney Transplantation: Novel Perspectives toward Biomarker Identification,” Online, BioMed Research International 2019:1602539; Wu, Q., Turturice, B., Wagner, S., Huang. Y., Gupta, P. K., and 9 others 2019. “Gut Microbiota Can Impact Chronic Murine Lung Allograft Rejection,” American Journal of Respiratory Cell and Molecular Biology 60(1):131-134; Weiner, D. E., Park, M., Tighiouart. H., Joseph, A. A., Carpenter, M. A., and 14 others 2019. “Albuminuria and Allograft Failure, Cardiovascular Disease Events, and All-Cause Death in Stable Kidney Transplant Recipients: A Cohort Analysis of the FAVORIT [Folic Acid for Vascular Outcome Reduction in Transplantation] Trial,” American Journal of Kidney Diseases 73(1):51-61; Eikmans, M., Gielis, E. M., Ledeganck, K. J., Yang, J., Abramowicz, D., and Claas, F. F. J. 2019. “Non-invasive Biomarkers of Acute Rejection in Kidney Transplantation: Novel Targets and Strategies,” Online, Frontiers in Medicine (Lausanne, Switzerland) 5:358; Muiru. A. N., Shlipak, M. G., Scherzer, R., Zhang, W. R., Ascher, S. B., and 12 others 2019. “Kidney Disease Risk Factors Associate with Urine Biomarkers Concentrations in HIV-positive Persons; a Cross-sectional Study,” Online, BioMed Central Nephrology 20(1):4; Jia, L., Jia. L., Fu, W., Jia. R., Wu, L., Li, X., Jia, Q., and Zhang, H. 2018. “Identification of Potential Key Protein Interaction Networks of B K Virus Nephropathy in Patients Receiving Kidney Transplantation,” (Nature Research) Scientific Reports 8(1):5017; Naesens, M. and Anglicheau, D. 2018. “Precision Transplant Medicine: Biomarkers to the Rescue,” Journal of the American Society of Nephrology 29(1):24-34; Wee, Y. M., Lee, H. W., Choi, M. Y., Jung, H. R., Choi, J. Y., and 5 others 2018. “A Composite of Urinary Biomarkers for Differentiating between Tubulointerstitial Inflammation and Interstitial Fibrosis/Tubular Atrophy in Kidney Allografts,” Annals of Hepato-Biliary-Pancreatic Surgery (Seoul, South Korea) 22(4):310-320; Honda, M., Sugawara, Y., Kadohisa, M., Shimata, K., Sakisaka, M., and 8 others 2018. “Long-term Outcomes of ABO-incompatible Pediatric Living Donor Liver Transplantation,” Transplantation 2018 102(10):1702-1709; Mysore, K. R., Himes, R. W., Rana. A., Teruya, J., Desai, M. S., and 13 others 2018. “ABO-incompatible Deceased Donor Pediatric Liver Transplantation: Novel Titer-based Management Protocol and Outcomes,” Pediatric Transplantation 22(7):e13263; Jia. R., Li, Y., Li, X., Hunt, E. B., Sullivan, A., Galvin, J., MacSharry, J., and Murphy, D. M. 2018. “Gastric Aspiration and Its Role in Airway Inflammation,” Open Respiratory Medicine Journal 12:1-10; Jia, Q., and Zhang, H. 2018. “LCK [lymphocyte kinase] as a Potential Therapeutic Target for Acute Rejection after Kidney Transplantation: A Bioinformatics Clue,” Journal of Immunology Research 2018:6451298; Xu, C. X., Shi, B. Y., Wang, C., Li, Q., and Li, J. 2018. “Gut Microbiota and Its Implications in Small Bowel Transplantation,” Frontiers of Medicine 12(3):239-248; Jin, Z. K., Hao. J. J., Duan, W. L., and 7 others 2018. “Multiple-biomarkers Provide Powerful Prediction of Early Acute Renal Allograft Rejection by Combination of Serum Fractalkine, IFN-γ [inerferon gamma] and IP-10 [interferon gamma-induced protein 10],” Transplant Immunology 50:68-74; Feng. S., Bucuvalas, J. C., Demetris, A. J., Burrell, B. E., Spain, K. M., and 21 others 2018. “Evidence of Chronic Allograft Injury in Liver Biopsies from Long-term Pediatric Recipients of Liver Transplants,” Gastroenterology 155(6):1838.e7-1851.e7; McIntosh, C. M., Chen, L., Shaiber, A., Eren, A. M., and Alegre, M. L. 2018. “Gut Microbes Contribute to Variation in Solid Organ Transplant Outcomes in Mice,” Microbiome 6(1):96; Guerra, M. R., Naini, B. V., Scapa, J. V., Reed, E. F., Busuttil, R. W., and 6 others 2018. “Obliterative Portal Venopathy: A Histopathologic Finding Associated with Chronic Antibody-mediated Rejection in Pediatric Liver Allografts,” Pediatric Transplantation 22(2); He. Y., Wen, Q., Yao. F., Xu. D., Huang. Y., and Wang, J. 2017. “Gut-Lung Axis: The Microbial Contributions and Clinical Implications,” Critical Reviews in Microbiology 43(1):81-95; Budden, K. F., Gellatly, S. L., Wood, D. L., Cooper, M. A., Morrison, M., Hugenholtz, P., and Hansbro, P. M. 2017. “Emerging Pathogenic Links between Microbiota and the Gut-Lung Axis,” Nature Reviews. Microbiology 15(1):55-63; Angeletti, A. and Cravedi. P. 2017. “Looking into the Graft without a Biopsy: Biomarkers of Acute Rejection in Renal Transplantation,” Contributions to Nephrology 190:181-193; Ix, J. H., Katz, R., Bansal, N., Foster, M., Weiner, D. E., and 9 others 2017. “Urine Fibrosis Markers and Risk of Allograft Failure in Kidney Transplant Recipients: A Case-cohort Ancillary Study of the FAVORIT [Folic Acid for Vascular Outcome Reduction in Transplantation] Trial,” American Journal of Kidney Diseases 69(3):410-419; Ma, Y., Pandzic, E., Nicovich, P. R., Yamamoto, Y., Kwiatek, J., and 4 others 2017. “An Intermolecular FRET [Forster resonance energy transfer] Sensor Detects the Dynamics of T Cell Receptor Clustering,” Nature Communications 8:15100; Clerkin, K. J., Ali, Z. A., and Mancini, D. M. 2017. “New Developments for the Detection and Treatment of Cardiac Vasculopathy,” Current Opinion in Cardiology February 15; Wei, J., Raynor, J., Nguyen, T. L., and Chi, H. 2017. “Nutrient and Metabolic Sensing in T Cell Responses,” Online, Frontiers in Immunology (Lausanne, Switzerland) 8:247; Chen, X., Zheng. J., Cai, J., Li, H., Li, S., and 9 others 2017. “The Cytoskeleton Protein β-actin May Mediate T Cell Apoptosis during Acute Rejection Reaction after Liver Transplantation in a Rat Model,” American Journal of Translational Research 9(11):4888-4901; Wozniak, L. J., Naini, B. V., Hickey, M. J., Bhattacharyya, S., Reed, E. F., and 5 others 2017. “Acute Antibody-mediated Rejection in ABO-compatible Pediatric Liver Transplant Recipients: Case Series and Review of the Literature,” Pediatric Transplantation 21(1); Kransdorf, E. P. and Kobashigawa, J. A. 2017. “Novel Molecular Approaches to the Detection of Heart Transplant Rejection,” Personalized Medicine 14(4):293-297; Shiu, K. Y., Mclaughlin, L., Rebollo-Mesa, I., Zhao, J., Burton, H., and 9 others 2017. “Graft Dysfunction in Chronic Antibody-mediated Rejection Correlates with B-cell-dependent Indirect Antidonor Alloresponses and Autocrine Regulation of Interferon-γ Production by Th1 Cells,” Kidney International 91(2):477-492; Salvadori, M. and Tsalouchos, A. 2017. “Biomarkers in Renal Transplantation: An Updated Review,” Online, World Journal of Transplantation (Pleasanton, California) 7(3):161-178; Salvadori, M. and Tsalouchos, A. 2017. “Biomarkers in Renal Transplantation: An updated review,” Online, World Journal of Transplantation (Pleasanton, California) 7(3):161-178; Jacquemont, L., Soulillou, J. P., and Degauque, N. 2017. “Blood Biomarkers of Kidney Transplant Rejection, an Endless Search?,” Expert Review of Molecular Diagnostics 17(7):687-697; Bestard, O. and Cravedi, P. 2017. “Monitoring Alloimmune Response in Kidney Transplantation,” Journal of Nephrology 30(2):187-200; Benzimra, M., Calligaro, G. L., and Glanville, A. R. 2017. “Acute Rejection,” Journal of Thoracic Disease 9(12):5440-5457; Zou, J., Duffy, B., Slade, M., Young, A. L., Steward, N.5., Hachem, R., and Mohanakumar, T. 2017. “Rapid Detection of Donor Cell Free DNA in Lung Transplant Recipients with Rejections Using Donor-recipient HLA Mismatch,” Human Immunology 78(4):342-349; Lei, Y. M., Chen, L., Wang. Y., Stefka, A. T., Molinero, L. L., and 8 others 2016. “The Composition of the Microbiota Modulates Allograft Rejection,” Journal of Clinical Investigation 126(7):2736-2744; Liu, Y., Blanchfield, L., Ma. V. P., Andargachew. R., Galior, K., and 3 others 2016. “DNA-based Nanoparticle Tension Sensors Reveal that T-cell Receptors Transmit Defined pN [piconewton] Forces to their Antigens for Enhanced Fidelity,” Online, Proceedings of the National Academy of Sciences of the United States of America 113(20):5610-5615; Perez, J. D., Sakata, M. M., Colucci, J. A., Spinelli, G. A., Felipe, C. R., and 6 others 2016. “Plasma Proteomics for the Assessment of Acute Renal Transplant Rejection,” Life Sciences 158:111-120; Christians, U., Klawitter, J., and Klawitter, J. 2016. “Biomarkers in Transplantation —Proteomics and Metabolomics,” Therapeutic Drug Monitoring 38 Supplement 1: S70-S74; Baroja-Mazo, A., Revilla-Nuin, B., Parrilla, P., Martinez-Alarcon, L., Ramirez, P., and Pons, J. A. 2016. “Tolerance in Liver Transplantation: Biomarkers and Clinical Relevance,” World Journal of Gastroenterology 22(34):7676-7691; Okubo, K., Wada, H., Tanaka, A., Eguchi, H., Hamaguchi, M., and 13 others 2016. “Identification of Novel and Noninvasive Biomarkers of Acute Cellular Rejection after Liver Transplantation by Protein Microarray,” Transplantation Direct 2(12):e118; Kim, N., Yoon, Y. I., Yoo. H. J., Tak, E., Ahn, C. S., and 3 others 2016. “Combined Detection of Serum IL-10, IL-17, and CXCL10 Predicts Acute Rejection following Adult Liver Transplantation,” Molecules and Cells 39(8):639-644; Starling, R. C., Stehlik, J., Baran, D. A., Armstrong, B., Stone, J. R., and 12 others 2016. “Multicenter Analysis of Immune Biomarkers and Heart Transplant Outcomes: Results of the Clinical Trials in Organ Transplantation-05 Study,” American Journal of Transplantation 16(1):121-136; Walls, J., Sinclair, L., and Finlay, D. 2016. “Nutrient Sensing, Signal Transduction and Immune Responses,” Seminars in Immunology 28(5):396-407; Nasr. M., Sigdel. T., and Sarwal, M. 2016. “Advances in Diagnostics for Transplant Rejection,” Expert Review of Molecular Diagnostics 16(10):1121-1132; Germani, G., Rodriguez-Castro, K., Russo, F. P., Senzolo, M., Zanetto, A., Ferrarese, A., and Burra, P. 2015. “Markers of Acute Rejection and Graft Acceptance in Liver Transplantation,” World Journal of Gastroenterology 21(4):1061-1068; Dieterlen, M. T., John, K., Bittner, H. B., Mende, M., Tarnok, A., Mohr, F. W., and Barten, M. J. 2015. “Assessment of Immunological Biomarkers in the First Year after Heart Transplantation,” Disease Markers 2015:678061; Raschzok, N., Reutzel-Selke, A., Schmuck, R. B., Morgul. M. H., Gauger, U., and 14 others 2015. “CD44 and CXCL9 Serum Protein Levels Predict the Risk of Clinically Significant Allograft Rejection after Liver Transplantation,” Liver Transplantation 21(9):1195-1207; Ramsay, G. and Cantrell, D. 2015. “Environmental and Metabolic Sensors that Control T Cell Biology,” Online, Frontiers in Immunology (Lausanne, Switzerland) 6:99; Huang, H., Xu. X., Yao. C., Cai, M., Qian, Y., Wang, X., and Shi, B. 2014. “Serum Levels of CXCR3 [chemokine receptor] Ligands Predict T Cell-mediated Acute Rejection after Kidney Transplantation,” Molecular Medicine Reports 9(1):45-50; Zhang, Q., Liu, Y. F., Su, Z. X., Shi, L. P., and Chen, Y. H. 2014. “Serum Fractalkine and Interferon-gamma Inducible Protein-10 Concentrations are Early Detection Markers for Acute Renal Allograft Rejection,” Transplantation Proceedings 46(5):1420-1425; McMinn, J. F., Lang, N. N., McPhadden, A., Payne, J. R., Petrie, M. C., and Gardner, R. S. 2014. “Biomarkers of Acute Rejection following Cardiac Transplantation,” Biomarkers in Medicine 8(6):815-832; Choudhuri, K., Llodra, J., Roth, E. W., Tsai, J., Gordo, S., and 4 others 2014. “Polarized Release of T-cell-receptor-enriched Microvesicles at the Immunological Synapse,” Nature 507(7490):118-123; Lo. D. J., Kaplan, B., and Kirk, A, D. 2014. “Biomarkers for Kidney Transplant Rejection,” Nature Reviews. Nephrology 10(4):215-225; Moreau, A., Varey, E., Anegon, I., and Cuturi, M. C. 2013. “Effector Mechanisms of Rejection,” Cold Spring Harbor Perspectives in Medicine 3(11). pii: a015461; Crescioli, C. 2013. “The Role of Immunological Biomarkers in Cardiac Rejection,” Current Opinion in Organ Transplantation 18(5):595-600; Verhelst, X. P., Troisi, R. I., Colle, I., Geerts, A., and van Vlierberghe, H. 2013. “Biomarkers for the Diagnosis of Acute Cellular Rejection in Liver Transplant Recipients: A Review,” Hepatology Research 43(2):165-178; Berastegui, C., Roman, J., Monforte, V., Bravo, C., Lopez-Meseguer, M., and 4 others 2013. “Biomarkers of Pulmonary Rejection,” Transplantation Proceedings 45(9):3163-3169; Cobbold, S. P. 2013. “The mTOR [mammalian target of rapamycin] Pathway and Integrating Immune Regulation,” Immunology 140(4):391-398; Labarrere, C. A. and Jaeger, B. R. 2012. “Biomarkers of Heart Transplant Rejection: The Good, the Bad, and the Ugly!,” Translational Research 159(4):238-251; Dengler, T. J., Gleissner, C. A., Klingenberg, R., Sack, F. U., Schnabel, P. A., and Katus, H. A. 2007. “Biomarkers after Heart Transplantation: Nongenomic,” Heart Failure Clinics 3(1):69-81).

When the condition of the patient allows, metered is preferable to sudden compound vascular bypass, or sudden switched-organ transplantation. To best take advantage of the immaturity of the immune system, maternal immune tolerance, and thwart the multiorgan impairment associated with abnormal, such as Fontan circulation, transplantation in a neonate (see, for example, van Well, G. T. J., Daalderop, L. A., Wolfs. T., and Kramer, B. W. 2017. “Human Perinatal Immunity in Physiological Conditions and During Infection,” Molecular and Cellular Pediatrics 4(1):4; Georgountzou. A. and Papadopoulos, N. G. 2017. “Postnatal Innate Immune Development: From Birth to Adulthood,” Online, Frontiers in Immunology (Lausanne, Switzerland) 8:957; Zachary, A. A. and Leffell. M. S. 2016. “HLA Mismatching Strategies for Solid Organ Transplantation-A Balancing Act,” Online, Frontiers in Immunology (Lausanne, Switzerland) 7:575; Fragiadakis, G. K., Baca, Q. J., Gherardini, P. F., Ganio, E. A., Gaudilliere, D. K., and 16 others 2016. “Mapping the Fetomaternal Peripheral Immune System at Term Pregnancy,” Journal of Immunology (Baltimore) 197(11):4482-4492; Kumar, S. K. and Bhat, B. V. 2016. “Distinct Mechanisms of the Newborn Innate Immunity,” Immunology Letters 173:42-54; Simon, A. K., Hollander, G. A., and McMichael, A. 2015. “Evolution of the Immune System in Humans from Infancy to Old Age,” Proceedings of the Royal Society of London. Biological Sciences 282(1821):20143085; PrabhuDas, M., Bonney, E., Caron, K., Dey. S., Erlebacher, A., and 12 others 2015. “Immune Mechanisms at the Maternal-fetal Interface: Perspectives and Challenges,” Nature Immunology 16(4):328-334; Schweiger, M., Stiasny, B., Dave, H., Cavigelli-Brunner, A., Balmer, C., and 4 others 2015. “Pediatric Heart Transplantation,” Journal of Thoracic Disease 7(3):552-559; Marchant, A. and Kollmann, T. R. 2015. “Understanding the Ontogeny of the Immune System to Promote Immune-mediated Health for Life,” Online, Frontiers in Immunology (Lausanne, Switzerland) 6:77; Basha, S., Surendran, N., and Pichichero, M. 2014. “Immune Responses in Neonates,” Expert Review of Clinical Immunology 10(9):1171-1184; Alijotas-Reig. J., Llurba, E., and Gris, J. M. 2014. “Potentiating Maternal Immune Tolerance in Pregnancy: A New Challenging Role for Regulatory T Cells,” Placenta 35(4):241-248; Gervassi, A., Lejarcegui, N., Dross, S., Jacobson, A., Itaya, G., and 4 others 2014. Myeloid Derived Suppressor Cells are Present at High Frequency in Neonates and Suppress in Vitro T Cell Responses,” Online, PLoS [Public Library of Science] One 9(9):e107816: Thrush, P. T. and Hoffman, T. M. 2014. “Pediatric Heart Transplantation-indications and Outcomes in the Current Era,” Journal of Thoracic Disease 6(8):1080-1096; Butts, R. J., Savage, A. J., Nietert, P. J., Kavarana, M., Moussa, O., Burette, A. L., and Atz, A. M. 2014. “Effect of Human Leukocyte Antigen-Cd -DQ Matching on Pediatric Heart Transplant Graft Survival,” Journal of Heart and Lung Transplantation (New York, New York) 33(12):1282-2387; Burt, T. D. 2013. “Fetal Regulatory T Cells and Peripheral Immune Tolerance in Utero: Implications for Development and Disease,” American Journal of Reproductive Immunology (New York) 69(4):346-358; Conway, J., Chin, C., Kemna, M., Burch, M., Barnes, A., and 6 others 2013. “Donors' Characteristics and Impact on Outcomes in Pediatric Heart Transplant Recipients,” Pediatric Transplantation 17(8):774-781; Maruyama, M. 2013. “Age-associated Decline in the Immune System,” (in Japanese with English abstract at Pubmed), Nihon Rinsho [Japanese Journal of Clinical Medicine] 71(6):993-998; Ygberg. S. and Nilsson, A. 2012. “The Developing Immune System—from Foetus to Toddler,” Acta Paediatrica (Oslo) 101(2):120-127; Ntrivalas, E., Levine, R., Kwong, C., Gilman-Sachs, A., and Beaman, K. 2010. “The a2 Isoform of Vacuolar ATPase is a Modulator of Implantation and Feto-maternal Immune Tolerance in Early Pregnancy,” Journal of Reproductive Immunology 85(1):106-111; Makrigiannakis, A., Karamouti, M., Drakakis, P., Loutradis, D., and Antsaklis, A. 2008. “Fetomaternal Immunotolerance,” American Journal of Reproductive Immunology 60(6):482-496; Kanellopoulos-Langevin, C., Caucheteux, S. M., Verbeke, P., and Ojcius, D. M. 2003. “Tolerance of the Fetus by the Maternal Immune System: Role of Inflammatory Mediators at the Feto-maternal Interface,” Reproductive Biology and Endocrinology 1:121) with an inadequately repairable heart is undertaken as early as possible. This may precede of succeed maternal haploidentical microchimerization tolerance.

Unless parentally chimerized to a degree considered significantly adverse during gestation, switch transplantation in a neonate is by the sudden rather than metered technique. Provided the replacement heart is a good match, other basic problems in pediatric heart transplantation-recipient pulmonary vascular resistance and/or vasculopathy before transplantation of the heart alone in lieu of a heart and lung transplant—is likely less problematic when not complicated by a significant antecedent interruption in perfusion (see, for example, Goldraich, L. A., Stehlik, J., Kucheryavaya, A. Y., Edwards. L. B., and Ross, H. J. 2016. “Retransplant and Medical Therapy for Cardiac Allograft Vasculopathy: International Society for Heart and Lung Transplantation Registry Analysis,” American Journal of Transplantation 16(1):301-309; Maxwell, B. G., Sheikh, A. Y., Ajuba-Iwuji, C. C., Heitmiller, E. S., and Vricella, L. A. 2015. “Pulmonary Vascular Resistance Index and Mortality after Paediatric Heart Transplant,” Cardiology in the Young (Cambridge, England) 25(6):1141-1147; Schweiger, M., Stiasny, B., Dave, H., Cavigelli-Brunner, A., Balmer, C., and 4 others 2015. “Pediatric Heart Transplantation,” Journal of Thoracic Disease 7(3):552-559; Moreira, N., Baptista, R., Costa, S., Franco, F., Pego, M., and Antunes. M. 2015. “Lowering Pulmonary Wedge Pressure after Heart Transplant: Pulmonary Compliance and Resistance Effect,” Arquivos Brasileiros de Cardiologia 105(3):292-300; Sobieszczańska-Małek, M., Zielinski, T., Piotrowski, W., Korewicki, J., and Of Polkard-Hf, O. B. 2014. “Prognostic Value of Pulmonary Hemodynamic Parameters in Cardiac Transplant Candidates,” Cardiology Journal 21(5):532-538; Guihaire, J., Mercier. O., Flecher, E., Aymami, M., Fattal. S., and 13 others 2014. “Comparison of Cardiac Allograft Vasculopathy in Heart and Heart-lung Transplantations: A 15-year Retrospective Study,” Journal of Heart and Lung Transplantation (New York, New York) 33(6):636-643; Conway, J., Manlhiot, C., Kirk, R., Edwards, L. B., McCrindle, B. W., and Dipchand. A. I. 2014, Op cit. above under section 2a(3), entitled Remotely Procured Graft Organ Preservation; Chiu, P., Russo, M. J., Davies. R. R., Addonizio, L. J., Richmond, M. E., and Chen, J. M. 2012. “What is High Risk? Redefining Elevated Pulmonary Vascular Resistance Index in Pediatric Heart Transplantation,” Journal of Heart and Lung Transplantation (New York, New York) 31(1):61-66; Gazit. A. Z. and Fehr. J. 2011. “Perioperative Management of the Pediatric Cardiac Transplantation Patient,” Current Treatment Options in Cardiovascular Medicine 13(5):425-443; Shaffer, K. M., Denfield, S. W., Schowengerdt, K. O., Towbin, J. A., Radovancevic, B., and 3 others 1998. “Cardiac Transplantation for Pediatric Patients. With Inoperable Congenital Heart Disease,” Texas Heart Institute Journal 25(1):57-63; Gajarski, R. J., Towbin, J. A., Bricker, J. T., Radovancevic, B., Frazier, O. H., and 3 others 1994. “Intermediate Follow-up of Pediatric Heart Transplant Recipients with Elevated Pulmonary Vascular Resistance Index,” Journal of the American College of Cardiology 23(7):1682-1627; Addonizio, L. J., Gersony, W. M., Robbins. R. C., Drusin, R. E., Smith, C. R., and 3 others 1987. “Elevated Pulmonary Vascular Resistance and Cardiac Transplantation,” Circulation 76(5 Part 2):V52-V55).

In addition to routine ultrasound and Doppler imaging, aberrant development of the heart and left ventricular dysfunction in utero is detectable through, n-terminal pro-B-type natriuretic peptide assay (Leufgen, C., Gembruch, U., Stoffel-Wagner, B., Fimmers. R., Merz, W. M. 2017 Op cit.; Carvajal, J. A., Ferrer, F. A., Araya, F. I., Delpiano, A. M. 2017, Op cit.; Merz, W. M., Leufgen, C., Fimmers, R., Stoffel-Wagner, B., and Gembruch, U. 2014 Op cit.; Merz, W. M., Kübler, K., Albers, E., Stoffel-Wagner, B., and Gembruch, U. 2012. “N-terminal Pro-B-type Natriuretic Peptide in the Circulation of Fetuses with Cardiac Malformations,” Clinical Research in Cardiology 101(2):73-79).

Initiated as soon as a replacement heart is found, preferably shortly following birth, both recipient and donor are immunologically primed, that is, cross-chimerized, in preparation for transfer of the replacement heart through blood and hematopoietic transfusions, irradiation as necessary, and if likely to prove further beneficial, a partial spleen and/or thymus allograft; the methods for averting immunological rejection, by now highly evolved, are no different in preparing for a solid organ allograft (homograft) by sudden switching than by conventional means.

Tolerance induction through cell therapy can be initiated prior to and continue during and following transplantation (see, for example, Deo, D., Marchioni, M., and Rao, P. 2022. “Mesenchymal Stem/Stromal Cells in Organ Transplantation, Online, MDPI [Multidisciplinary Digital Publishing Institute] Pharmaceuties (Basel, Switzerland) 14(4):791; Fortunato, M., Morali, K., Passeri, L., and Gregori, S. 2021. “Regulatory Cell Therapy in Organ Transplantation: Achievements and Open Questions,” Online, Frontiers in Immunology (Lausanne, Switzerland) 12:641596; Hoogduijn. M. J. Issa, F., Casiraghi, F., and Reinders, M. E. J. 2021. “Cellular Therapies in Organ Transplantation,” Online, Transplant International 34(2):233-244; Pilat, N., Lefsihane, K., Brouard, S., Kotsch, K., Falk. C., and 6 others 2021. “T- and B-cell Therapy in Solid Organ Transplantation: Current Evidence and Future Expectations,” Online, Transplant International 34(9):1594-1606; Shaw, B. I., Ord. J. R., Nobuhara. C., and Luo, X. 2021. “Cellular Therapies in Solid Organ Allotransplantation: Promise and Pitfalls,” Online, Frontiers in Immunology (Lausanne, Switzerland) 12:714723; Zheng, Q., Zhang, S., Guo, W. Z., and Xiao-Kang. L. 2021. “The Unique Immunomodulatory Properties of MSC [mesenchymal stem cell]-derived Exosomes in Organ Transplantation,” Online, Frontiers in Immunology (Lausanne, Switzerland) 12:659621; Almeida-Porada, G., Atala, A. J., and Porada, C. D. 2020. “Therapeutic Mesenchymal Stromal Cells for Immunotherapy and for Gene and Drug Delivery,” Online, Molecular Therapy Methods and Clinical Development 16:203-224; Podesta, M. A., Remuzzi, G., and Casiraghi, F. 2020. “Mesenchymal Stromal Cell Therapy in Solid Organ Transplantation,” Online, Frontiers in Immunology (Lausanne, Switzerland) 11:618243; Cai, S. and Chandraker, A. 2019. “Cell Therapy in Solid Organ Transplantation,” Current Gene Therapy 19(2):71-80; Nguyen, M. N., Nayemama, A., Jones, S. C., Kasamon, Y. L., and Waldron, P. E. 2019. “Solid Organ Transplant Rejection Associated with the Use of the Immunomodulatory Drugs (IMIDs),” Online, Blood 134 (Supplement 1):2189; Pool. M., Leuvenink, H., and Moers, C. 2019. “Reparative and Regenerative Effects of Mesenchymal Stromal Cells—Promising Potential for Kidney Transplantation?,” Online, International Journal of Molecular Science 20(18):4614; Kaundal. U., Bagai, U., and Rakha, A. 2018. “Immunomodulatory Plasticity of Mesenchymal Stem Cells: A Potential Key to Sucessful Solid Organ Transplantation,” Online, Journal of Translational Medicine 16:31; Safinia, N., Grageda, N., Scotta, C., Thirkell, S. Fry, L. J., and 4 others 2018. “Cell Therapy in Organ Transplantation: Our Experience on the Clinical Translation of Regulatory T Cells,” Online, Frontiers in Immunology (Lausanne, Switzerland) 9:354; Garakani, R. and Saidi, R. F. 2017. “Recent Progress in Cell Therapy in Solid Organ Transplantation,” Online, International Journal of Organ Transplantation Medicine 8(3):125-131; Hartleif. S., Schumm, M., Doring, M., Mezger, M., Lang. P., and 6 others 2017. “Safety and Tolerance of Donor-derived Mesenchymal Stem Cells in Pediatric Living-donor Liver Transplantation: The MYSTEP1 [Mesenchymal Stem Cells in Pediatric Living-donor Liver Transplantation 1] Study,” Online, Stem Cells International 2017:2352954; Argani, H. 2016. “Cell Therapy in Solid Organ Transplant,” Experimental and Clinical Transplantation 14 (Supplement 3):6-13; McDonald-Hyman, C., Turka, L. A., and Blazar, B. R. 2015. “Advances and Challenges in Immunotherapy for Solid Organ and Bone Marrow Transplantation,” Online, Science Translational Medicine 7(280):280rv2; Soeder, Y., Loss, M., Johnson, C. L., Hutchinson, J. A., Haarer, J., and 7 others 2015. “First-in-human Case Study: Multipotent Adult Progenitor Cells for Immunomodulation after Liver Transplantation,” Online, Stem Cells Translational Medicine 4(8):899-904; English, K., and Wood, K. J. 2013. “Mesenchymal Stromal Cells in Transplantation Rejection and Tolerance,” Online, Cold Spring Harbor Perspectives in Medicine 3(5):a015560; Franquesa, M. Hoogdujin, M. J., Reinders. M. E., Eggenhofer, E., Engela, A U., and 36 others 2013, Mesenchymal Stem Cells in Solid Organ Transplantation (MiSOT) Fourth Meeting: Lessons Learned from First Clinical Trials,” Online, Transplantation 96(3):234-238; Moreau, A., Varey, E., Anegon, I., and Cuturi, M.-C. 2013. “Effector Mechanisms of Rejection,” Online, Cold Spring Harbor Perspectives in Medicine 3(11):a015461; Pileggi, A., Xu X., Tan, J., and Ricodi, C. 2013. “Mesenchymal Stromal (Stem) Cells to Improve Solid Organ Transplant Outcome: Lessons from the Initial Clinical Trials,” Online, Current Opinion in Organ Transplantation 18(6):672-681; Dahlke, M. H. 2012. “Immunomodulative Cell Therapy after Solid Organ Transplantation: Which Cell Type When?,” Current Opinion in Organ Transplantation (Hagerstown, Maryland) 17(1):33).

The effect of attaining postnatal immunity once normal tolerance or suppression for the mother subsides not predictable with confidence, no reliance is placed upon mother-fetus microchimerism to protect the fetus from infection. The procedure is best performed in utero through a hysterotomy (uterotomy). With or without the aid of a robot, to facilitate surgical precision and minimize operating time midgestation should not require an open procedure whereby the fetus is temporarily removed through the hysterotomy with placenta and with tocolytics to suppress premature labor before return to the uterus to resume normal gestation (see, for example, Hoagland, M. A. and Chatterjee, D. 2017. “Anesthesia for Fetal Surgery,” Paediatric Anaesthesia 27(4):346-357; Saxena, K. N. 2009. “Anaesthesia for Fetal Surgeries,” Indian Journal of Anaesthesia 53(5):554-559; Estes, J. M., MacGillivray, T. E., Hedrick, M. H., Adzick, N. S., and Harrison, M. R. 1992. “Fetoscopic Surgery for the Treatment of Congenital Anomalies,” Journal of Pediatric Surgery 27(8):950-954). Provided gestation is far enough along, labor induction may be indicated; however, unless a replacement heart could not be found earlier, a heart transplant this late is avoided.

Solenoid-driven ductus side-entry diversion valve-jackets implement not only sudden compound vascular bypass, or sudden switched-organ transplantation, and vascular servovalves implement not only metered transplantation, but provide accessory channels to directly target drugs to the treatment site or sites thus more effectively warding off rejection or deterioration (see, for example, da Silva, M. B., da Cunha, F. F., Terra, F. F., and Camara, N. O. 2017. “Old Game, New Players: Linking Classical Theories to New Trends in Transplant Immunology,” Online, World Journal of Transplantation (Pleasanton, California) 7(1):1-25; Ravindranath, M. H. 2017, Op cit. above under 1. Background of the Invention. a. General; Richmond, M. E., Easterwood, R., Singh, R. K., Gilmore, L., Beddows, K., and 4 others 2016. “Low-dose Donor Dopamine is Associated with a Decreased Risk of Right Heart Failure in Pediatric Heart Transplant Recipients,” Transplantation 100(12):2729-2734; Schumacher, K. R., Almond, C., Singh, T. P., Kirk, R., Spicer. R., and 7 others 2015. “Predicting Graft Loss by 1 year in Pediatric Heart Transplantation Candidates: An Analysis of the Pediatric Heart Transplant Study Database,” Circulation 131(10):890-898).

Turning now to FIG. 15A in which the vessels of the recipient, schematically shown as summarized on the right-hand side and those of the donor on the left-hand side, are depicted as singular for clarity, with all jacket-mainline-jacket connections between counterpart vessels, so that all inflowing vessels are represented above (to include the inferior vena cava) and all outflowing vessels are shown below (to include the pulmonary artery), the donor also having mainline connected jackets positioned at a small interval outside these to allow direct connection for continued machine support to preserve unused organs, the procedure for transplanting a solid organ, here exemplified in a beating heart transplantation in a fetus, may be summarized as follows:

In Step I, the chests of donor and recipient are opened and damped push-pull solenoid-actuated endovascular valve type side-entry diversion switch jackets are positioned on the vessels of both donor and recipient hearts, the chutes fully retracted so that blood flow continues uninterruptedly through the hearts and circulatory systems of donor and recipient as mutually exclusive, that is, with no blood passed from the recipient to the donor.

So that the donor can be continued on life support to preserve other organs for transplantation, the heart inflow and outflow vessels are additionally side-entry intravascular valve diversion switch jacketed above (craniad to) and below (caudad to) the transplantation circuit jackets, likewise to allow donor-recipient exclusive blood flow, the pair respective of each vessel connected together by a common mainline. Once the heart is removed, these will allow returning the diversion chutes in the jackets to the retracted position so that blood will flow directly from the inflow to the outflow vessels past the previously interposed heart.

1

The donor jackets are inverted relative to those on the recipient. In a total orthotopic transplant, the atria are not transected, so that there is no leakage directly from the atria. In a fetus or neonate, to allow larger jackets eventually to be placed during growth, the vessels of the donor heart are trimmed back and transected to take the heart with stumps of maximum length. To minimize the length of the lines connecting the two, donor and recipient should be as close beside one another as does not interfere with free movement of the surgical team. Both mainlines connecting the respective vessels of donor and recipient and accessory channels used to target medication into the lines and jackets consist of highly elastic coiled plastic tubes which spontaneously adopt the smallest length their attachments will allow without change in diameter.

This serves to reduce the length and space taken up by the lines when the new heart is placed in the chest of the recipient. In a fetus or a neonate, these self-contracting lines cooperate with the foam lining and spring hinges of the jackets to accommodate considerable growth, allowing the patient to fully adapt to the transplant and generally stabilize without the need for the vessels to be anastomosed as not essential for normal growth. Unlike the spring hinges used in the outlet half of inline coupling jackets, described below in section 2b(2)(c)(c1), entitled Inline Coupling Jackets Proper, these are not damped.

In an adult, anastomosis can likewise be dispensed with indefinitely, which is recommended in the elderly and frail patient, at the same time eliminating the need for a second open procedure. Placement of the diversion switch jackets along the inflow and outflow vessels of the donor and recipient, each with at least one accessory channel to be led as next addressed, and in a fetus of neonate, deferred until shortly before discharge, to the surface port, is by means of the technique described in U.S. Pat. No. 11,759,186, entitled Ductus Side-entry Jackets and Prosthetic Disorder Response Systems.

When the sudden circulation diversion switch controlling all of the diversion jackets in synchrony is depressed, the return of blood from the donor heart-now incorporated into the circulatory system of the recipient-flow with the chutes reversed and inverted compared to those of the stumps of the recipient following excision of his diseased heart. If delivery is to be through the birth canal, the mainlines through which the fetal recipient blood will be switched to the donor heart and accessory channel lines of the fetus and mother emerge through the integument as would a conventional central line, the differences consisting of passage through the amniotic sac and the fact that several lines involved, each must be clearly tagged for positive identification.

In this, transcutaneous (transdermal) line passage has the advantage of requiring shorter lines. If delivery will be by Cesarian section using the ex utero intrapartum treatment (citations above) as necessary so that the birth canal need not be kept clear, the lines are led out through the vagina, care taken to assure that nowhere do these kink. In a fetus, the surface port is not placed until shortly before discharge, the interval gauged to allow the patient to fully stabilize and confirm that the home caregiver and preferably at least one backup, understands the use of the accessory channel openings in the port. After about two months, any components not placed with the port are placed. Caregivers are provided with a drawing of the port with each opening labeled, with the schedule for its use and measures to be taken should each any more likely exigent eventuality arise annotated. Placement of the surface port is accomplished under local anesthesia.

The circulation of the fetal recipient is driven by his own heart with medicinal and support other than extracorporeal membrane oxygenation as necessary. So that damaged blood does not enter into the circulation of the recipient, the circulatory system of the donor is driven thus, his blood immediately replaced upon switching by the blood of the recipient. The mainlines, unused at this point, are connected between the respective counterpart heart vessel pairs of the two. If necessary, jackets had been placed on the heart vessels of the donor to target medication in a discriminatory manner to each vessel through one or more accessory channels. This should rarely prove the case, support of the donor heart other than by machine accomplished medically by venous infusion of intramuscular injection. When the plunger switch is depressed to fully advance all diversion chutes into the lumina, the circulation of the recipient is redirected from his defective heart to and through the heart of the donor.

Using the procedure delineated, the cardionector (cardionecteur, conduction system of the heart) is never subjected to ischemia and never manipulated as might induce a block. For these reasons, the risk of a synchronization problem during or following transplantation is much reduced, and therewith a significant cause of mortality (see, for example, Marzoa-Rivas, R., Perez-Alvarez, L., Paniagua-Martin, M. J., Ricoy-Martinez, E., Flores-Rios, X., and 9 others 2009. “Sudden Cardiac Death of Two Heart Transplant Patients with Correctly Functioning Implantable Cardioverter Defibrillators,” Journal of Heart and Lung Transplantation (New York, New York) 28(4):412-414; Calzolari, V., Angelini, A., Basso, C., Livi, U., Rossi, L., and Thiene, G. 1999. “Histologic Findings in the Conduction System after Cardiac Transplantation and Correlation with Electrocardiogramdings,” American Journal of Cardiology 84(6):756-759, A9). Nevertheless, if the risk of a post-transplantation dysrhythmia is judged significant, a cardioverter-defibrillator is implanted in the healthy donor heart before it is removed.

In Step II, the operator or an assistant depresses the switch that simultaneously advances the solenoid-driven diversion chutes from fully retracted to fully inside the lumina of both hearts. This fully diverts the continued flow of blood through the great vessels (heart, aorta, main pulmonary artery and pulmonary veins, and inferior venae cavae) from the defective fetal heart, and redirects this flow to and through the heart of the donor, effectively incorporating the donor heart into the circulatory system of the recipient. As the blood of the recipient arrives into the atria of the donor heart, the blood of the donor has been substantially emptied therefrom. Precise synchronization with the next systole is not essential, however, as the absolute volume of machine-damaged donor blood admixed into the following systole is far too slight to cause harm.

If believed advantageous switching is synchronized to an end-systole. Using a synchronization circuit, switching can be automated; however, the exact moment of switching is not critical. If the donor had been on prolonged machine support, switching to the new heart should be with the inflow momentarily closed just following a systole with the outlets open, at which moment most of the donor's blood will have been expelled into his own body. Immediately thereafter, the valves are switched, closing off the donor's heart from his own circulation and placing it in the circulatory system of the recipient.

The circulation of the recipient now suddenly redirected from his own diseased heart to that of the donor, the diversion jacket chutes are retracted and the jackets connected together so that blood flows directly from the inflowing to the outflowing vessels, maintaining the donor under continued machine support to preserve other organs for transplantation. The healthy heart of the donor now drives the circulation of the recipient with the blood of the recipient, which has never been subjected to the hemolytic trauma and/or dispersion of microemboli produced by a cardiopulmonary pump.

In Step III, the blood of the recipient having fully supplanted the donor and heart vessels, the defective heart and heart vessels of the recipient are removed. The donor body is not removed from machine support but kept perfused to preserve other organs for transplantation, periodic blood transfusions administered to replace blood cells damaged and detritus produced by the roller or peristaltic pump.

In Step IV, the heart of the donor with the jackets left in position on the stumps are orthotopically positioned in the chest of the recipient. Anastomosis of the great vessels is not essential for normal growth. Stabilization against the abrupt and forceful pumping action not provided by anastomosis to the donor atria and vessel stumps is provided by encasement within the central compartment of the mediastinum and compliant surrounding tissues. If and only if necessary, instability is countered by the deliberate creation of adhesions and/or by cardiac fixation amid the surrounding tissue, primarily to the posterior wall of the chest, without the removal of any lines.

If clinical judgment is to effect anastomosis immediately—of which the connection will contribute stabilization (see, for example, Goss, C. M. 1966 (ed.). Gray's Anatomy of the Human Body, Philadelphia, Pennsylvania: Lea and Febiger, page 543) but afford less flexibility in positioning of the heart—the mainlines connecting the jackets on both donor and recipient stumps left in place during stitching to bypass blood around the suture lines, the side connectors to which the mainlines were connected capped off, but the jackets with accessory channels left in place to directly target immunosuppressant, antimicrobial, and anti-inflammatory drugs directly to the treatment sites. As always, the suture lines are carefully observed for extravasation.

All drugs have the potential to induce harmful side effects, direct targeting having the fundamental advantages of allowing the direct dose to be more concentrated without affecting other tissue and the systemic dose to be considerably reduced. This is no less true for any junction created in cardiac or any other surgery, whether of stretched woven polyethylene terephthalate (Dacron®, Terylene®, woven Dacron® or Terylene®, bovid, or porcine sheet or tissue-tissue anastomoses. While the neonate remains in the clinic, all drug administration and monitoring is accomplished by conventional means, a port at the body surface, reservoirs, pumps, controller, or sensors, and so on are not placed until an interval preceding discharge sufficient to confirm that home caretakers fully understand their use.

In Step V, mother and fetus are closed and transferred to the intensive care unit.

Postprocedural steps VI nd VII consist of follow-up and placement of the body surface port.

In Step VI, the patient is closely monitored for the need for total lymphoid irradiation and antithymocyte globulin to avert a hyperacute rejection (see, for example, Vendramin, A., Gimondi, S., Bermema, A., Longoni, P., Rizzitano, S., Corradini, P., and Carniti, C. 2014. “Graft Monocytic Myeloid-derived Suppressor Cell Content Predicts the Risk of Acute Graft-versus-host Disease after Allogeneic Transplantation of Granulocyte Colony-stimulating Factor-mobilized Peripheral Blood Stem Cells,” Biology of Blood and Marrow Transplantation 20(12):2049-2055; Lan, F., Zeng, D., Higuchi, M., Higgins, J. P., and Strober, S. 2003, Op cit.; additional reference above in section 2, entitled Background of the Invention).

Step VII consists of placing a surface port, microcontroller, automatic drug delivery components such as drug reservoirs, outlet pumps or drip stopcocks, and sensors as necessary and attaching the accessory channels their respective openings in the surface port. In a neonate or infant, a prosthetic disorder response system as addressed in U.S. Pat. No. 11,759,186, entitled Ductus Side-entry Jackets and Prosthetic Disorder Response Systems is used to target immunosuppressant medication and chimerizing agents to the transplant organ, here the heart. Since the heart is replaced before the hypoxia of the defective congenital heart might have affected other organs, the treatment of comorbid disease is not involved.

However, targeted immunosuppressants administered with a relatively small background systemic dose, systemic chimerizing agents, and where the potential for other organs to require treatment, the targeted delivery of drugs pertinent thereto, justify the placement of a multichannel automatic adaptive hierarchical ambulatory prosthetic disorder response control system as described in U.S. Pat. No. 11,759,186. This differs from such use in an adult, who will have sustained degeneration of other organs and requires the coordinated delivery of immunosuppressant to the heart, lungs, kidneys, liver, and other organs or tissue as necessary to affect the component organs for the reinstatement of homeostasis.

While chimerizing agents are generally released into the systemic and pulmonary circulation, immunosuppressants are tightly targeted to avert the familiar and severe side effects such as rapid cell reproduction death seen in the loss of hair and nausea, among several others. Targeting is from the implanted reservoirs to the side-entry jackets bounding the transplant organ. The jacket on the supply artery or arteries deliver the locally effective immunosuppressant or immunosuppressants, and those on the veins deliver immunosuppressant reversal agents as necessary, systemic immunosuppressants released into the general circulation.

For example, methotrexate, an immunosuppressant and anti-cancer drug, and fluorouracil an anti-cancer drug are reversed by calcium leucovorin (folinic acid), doxorubicin and daunorubicin are anti-cancer drugs reversed by dexrazoxane, cyclophosphamide, an immunosuppressive, and ifosfamide, an anti-cancer drug used primarily for cancers of the lower urinary tract, are reversed by mesna. These two types of drugs are related in various ways, but primarily in that immunosuppression can expedite the development of cancer.

Reversal agents for immunosuppressants are several (see, for example, Chan, B. S., Dawson, A. H., and Buckley, N. A. 2017. “What Can Clinicians Learn from Therapeutic Studies about the Treatment of Acute Oral Methotrexate Poisoning?,” Clinical Toxicology (Philadelphia) 55(2):88-96; Bidaki, R., Kian, M., Owliaey, H., Babaei Zarch, M., and Feysal, M. 2017. “Accidental Chronic Poisoning with Methotrexate; Report of Two Cases,” Emergency (Tehran) 5(1):e67; Liew, D., Keith, C., Booth, J., and Perera, D. 2017. “Fatal Azathioprine Toxicity,” Australian Prescriber 40(3):109; Tafazoli, A. 2015. “Accidental Overdose of Oral Cyclosporine in Haematopoietic Stem Cell Transplantation: A Case Report and Literature Review,” Drug Safety-Case Reports 2(1):20; Weidmann, A., Foulkes, A. C., Kirkham, N., and Reynolds, N. J. 2014. “Methotrexate Toxicity during Treatment of Chronic Plaque Psoriasis: A Case Report and Review of the Literature,” Dermatology and Therapy (Heidelberg) 4(2):145-156; Rattu, M. A., Shah, N., Lee, J. M., Pham, A. Q., and Marzella, N. 2013. “Glucarpidase (Voraxaze), a Carboxypeptidase Enzyme for Methotrexate Toxicity,” Pharmacy and Therapeutics 2013 38(12):732-744; Griffith, B. P., Hardesty, R. L., Lee, A., Trento, A., Thompson, M. E., Shapiro, A. P., and Bahnson, H. T. 1985. “Management of Cyclosporine Toxicity by Reduced Dosage and Azathioprine,” Journal of Heart Transplantation 4(4):410-413).

Regardless of the drug overdosed, the most usual remedy for large systemic overdoses is withdrawal, making it clear that a minute amount not be taken up within the intended treatment site would rarely if ever induce injury, even without an outlet jacket to release a reversal agent. The jackets have a sufficient thickness of foam lining and spring hinges that open to allow for growth over a significant interval, allowing for many years of growth when placed in a neonate or infant, perhaps without the need for replacement even into adulthood.

The lines can be given as much slack as does not present a risk of ensnaring an organ or tissue. However, even though the components allow for some growth, the rate thereof during the early period will necessitate the replacement of jackets and lines endoscopically under local anesthesia, although at progressively longer intervals. About a half year following the transplantation, the original jackets and lines must be replaced.

Jacket replacement to accommodate growth is by removing the existing jacket, taking up the small amount of extravasated blood with an aspiration hose, placing a drop of low molecular weight heparin or heparinoid over the trepan opening, wrapping around the ductus at the level of the opening with a paper-thin strip of polyglycolic acid-based absorbable sheet stock containing the hole for the new larger diameter trepan, positioning the hole off to a side or distance about the circumference of the ductus as results in the new jacket being positioned at an angle that does not encroach upon surrounding tissue, and bonding the overlapped edges on the side opposite the opening with a drop of surgical cyanoacrylate cement.

Placement of the new jacket is accomplished with the aid of the water jacket to restrain blood from leaking as described in U.S. Pat. No. 11,759,186. Just as the original jacket, the replacement jacket incorporates a water jacket which remains after placement for use to target drugs to the treatment site through an accessory channel. Thus, such a jacket is used even if the loss of blood is small enough that the water jacket need not be used to restrain blood from leaking out through the trepan hole. The amount of blood that will leak without restraint is directly proportional to the size of the substrate vessel.

Responsive to exigent need, compound vascular bypass double heart transplantation in a prenate is addressed below in section 2b(1)(b)(b5), entitled Heterotopic Double Heart Metered Compound Vascular Bypass, or Switched-organ Transplantation [and so on], and the Use of Unified Entry Arterial and Venous Exit Confluence Conduits to Avoid Disuse Atrophy when the Graft Organ is Positioned in the Lower Abdomen by Direct Aortocaval Connection, in the subsection at the end thereof entitled Heterotopic Double Heart Transplantation in a Prenate, Neonate, or Infant. In developed countries which permit abortion, the pool of available replacement hearts can be made substantial, eliminating such need.

2b(1)(b)(b3). Neonatal and Infant Compound Vascular Bypass, or Switched-Organ Transplantation of the Heart

As with patients at all ages, the primary problem with heart transplantation in the very young is the lack of replacement hearts, with the secondary problem, the increased complexity of the tiny anatomy, which microsurgical, must be performed under a binocular microscope using tiny vascular servovalves. The tertiary problem, immune intolerance is usually alleviated due to the immaturity of both donor and recipient. For a procedure that involves the implantation of nonbiological components, another potential problem is growth. The immune system in the neonate and infant remains immature to allow the application of the compound vascular bypass sudden switch technique with the use of the device shown in FIGS. 15C and 15D as elaborated upon in the preceding section and referenced shortly below.

In prospective late prenatal, neonatal, infant, and toddler compound bypass heart transplantation, grouped vessel inlet/outlet confluence conduits as shown in FIGS. 15C and 15D addressed below in section 2b(1)(b)(b14), entitled Confluence Conduits: Connection of One or More Bloodlines to Flow through a Common Channel to the Same or a Different Number of Bloodlines with Self-leveling Apportionment of Outflow, in the subsection thereof entitled Tiny Vessel Grouped Inlet/Outlet Expandable Confluence Conduits, and in section 6, entitled Description of the Preferred Embodiments of the Invention, significantly reduce the duration of the operation for the already impaired patient thus improving the likelihood for a favorable outcome.

Relief from the first limitation would be accomplished were state laws changed to require that where abortion is permitted, the fetus must be provided to an organ transplant medical authority. The pool can also be expanded by reasonably liberalizing the zone of acceptability for transplantation to include organs with curable or suppressible disease, and those slightly larger in size, hence, in ejection fraction or left ventricular absolute stroke volume. Only a graft organ with a donor-recipient immune discord which cannot be satisfactorily suppressed with the aid of the continual monitoring and release of medication when detected by an implanted disorder response system should be disqualified.

Treatment of a neonate or infant with complete or dextro-transposition or discordance of the great arteries and associated cardiac defects severe as to justify a heart transplant but too frail to undergo such a procedure is addressed below in sections 2b(2)(a), entitled Transectionless and Ischemia-free Correction of Complete, or Dextro-transposition, of the Great Arteries, and 2b(2)(b), entitled Bidirectional Valves for Perinatal Extracardiac Correction of Complete or Dextro-transposition of the Great Arteries and Reciprocal Cross-circulation.

The need for a heart or a heart-liver transplant secondary to sustained Fontan circulation is addressed below in section 2b(1)(b)(b7), entitled Metered Compound Vascular Bypass, or Switched-organ Liver or Heart-Liver Transplantation. The measures delineated in section 2b(1)(b)(b5), entitled Heterotopic Double Heart Metered Compound Vascular Bypass, or Sudden Switched-organ, Transplantation [and so on], considerably expand the zone for acceptance of replacement hearts.

In a neonate with complete or dextro-transposition or discordance of the great arteries and associated cardiac defects severe as to justify a heart transplant but too frail to undergo such a procedure, vascular valves, along with transcatheteric or transluminal methods, offer a bridging strategy pending a heart transplant if still necessary once the baby has been allowed to gain in strength, or were pulmonary hypertension likely to result, a heart and lung transplant. Incision confined to gaining access, the opening required is much smaller and the procedure most often laparoscopic.

In any organ transplantation, the follow-up release of medication automatically by the totally implanted ambulatory prosthetic disorder response system is a significant factor predisposing to a successful outcome. The tiny size of the heart in a neonate and infant makes onerous the development of mechanical means other than extracorporeal membrane oxygenation, to bridge to a new heart. In a prenate or neonate with a heart so malformed that it cannot be repaired to the extent that it would provide normal circulation, replacement with a living heart transplanted orthotopically, that is, in the chest, is distinctly superior to any alternative resolution.

Instituted over four decades ago, neonatal heart transplantation . . . “for complex-uncorrectable heart disease . . . ” has allowed “ . . . unparalleled long-term survival, better than for any other form of solid-organ transplantation,” (John, M. and Bailey, L. L. 2018. Op cit. in section 2a, entitled Background of the Invention. General)(see also, Licata, J., Roberts, S. M., Janicki, P., and Bezinover, D. 2022. “Adult Renal Transplantation in a Patient 28 Years after Heart Transplantation as a Neonate for Hypoplastic Left Heart Syndrome,” Online, Case Reports in Transplantation 2022:7532199; Lin. Y., Davis, T. J., Zorrilla-Vaca, A. Wojcik, B. M., Miyamoto, S. D., and 4 others 2021. “Neonatal Heart Transplant Outcomes: A Single Institutional Experience,” Journal of Thoracic and Cardiovascular Surgery 162(5):1361-1368; John, M. M., Razzouk, A. J., Chinnock, R. E., Bock, M. J., Kuhn, M. A., Marterns. T. P., and Bailey, L. L. 2019. “Primary Transplantation for Congenial Heart Disease in the Neonatal Period: Long-term Outcomes,” Annals of Thoracic Surgery 108(6):1857-1864; Chinnock, R. E. and Bailey, L. L. 2011. “Heart Transplantation for Congenital Heart Disease in the First Year of Life,” Online, Current Cardiology Reviews 7(2):72-84).

Neonatal heart transplantation differs in three primary respects from prenatal heart transplantation:
1. The baby is no longer parasitically dependent upon and contained within the mother, eliminating removal from and return to the uterus while still a fetus as impediments.
2. The state in development of the heart is more highly differentiated, allowing connections to be made between corresponding vessels of the donor and recipient which if placed earlier would interfere with the ensuing differentiation in the structures where these had been jacketed (see, for example, Hikspoors, J. P. J. M., Kruepunga, N., Mommen, G. M. C., Kohler, S. E., Anderson, R. H., and Lamers. W. H. 2022. “A Pictorial Account of the Human Embryonic Heart between 3.5 and 8 Weeks of Development,” Online, Communications Biology 5:226; Mathew; P. and Bordoni, B. 2022. “Embryology, Heart,” Online, Treasure Island, Florida: StatPearls Publishing Co.; Faber, J. W., Eymann, S. 2021. “The Tube that Becomes the Human Heart,” Online, Transonic Systems Inc., Ithaca, New York, United States; Farraj; K. L. and Zeltser, R. 2021. “Embryology, Heart Tube,” Online, Treasure Island, Florida: StatPearls Publishing Co.; Hagoort, J., Moorman, A. F. M., Christoffels. V. M., and Jensen, B. 2021. “Quantified Growth of the Human Embryonic Heart,” Online, Biology Open 10(2):bio0570591; Houyel. L., and Meilhac, S. M. 2021. “Heart Development and Congenital Structural Heart Defects,” Online, Annual Review of Genomics and Human Genetics 22:257-284; Mathew, P. and Bordon, B. 2021. “Embryology, Heart,” Online, Treasure Island, Florida: StatPearls Publishing Co.; Buijtendijk, M. F. J., Barnett, P., and van den Hoff, M. J. B. 2020. “Development of the Human Heart,” Online, American Journal of Medical Genetics. Part C. Seminars in Medical Genetics 184(1):7-22; Ozawa, K., Davey, M. G., Tian, Z., Homick, M. A., Mejaddam, A. Y., and 3 others 2020. “Fetal Echocardiographic Assessment of Cardiovascular Impact of Prolonged Support on EXTrauterine Environment for Neonatal Development (EXTEND) System,” Online, Ultrasound in Obstetrics and Gynecology 55(4):516-522; Tan. C. M. J., and Lewandowski, A. J. 2020. “The Transitional Heart: From Early Embryonic and Fetal Development to Neonatal Life,” Online, Fetal Diagnosis and Therapy 47(5):373-386; Kidokoro, H., Yonei- Tamura, S., Tamura, K., Schoenwolf, G. C., and Saijoh, Y. 2018. Online, “The Heart Tube Forms and Elongates through Dynamic Cell Rearrangement Coordinated with Foregut Extension,” Development 145(7):dev152488; Sun, C. and Kontandis, M. I. 2018. “Physiology of Cardiac Development: From Genetics to Signaling to Therapeutic Strategies,” Online, Current Opinion in Physiology 1:123-139; Kloesel, B., DiNardo, J. A., and Body, S. C. 2016. “Cardiac Embryology and Molecular Mechanisms of Congenital Heart Disease—A Primer for Anesthesiologists,” Online, Anesthesia and Analgesia 123(3):551-569; Sylva, M., van den Hoff, M. J. B., and Moorman, A. F. M. 2014. “Development of the Human Heart,” American Journal of Medical Genetics Part A Human Genetic Disorders 164(6):1347-1371; Schleich, J.-M. 2002. “Images in Cardiology. Development of the Human Heart: Days 15-21,” Online, Heart 87(5):487; Friedman, A. H. and Fahey, J. T. 1993. “The Transition from Fetal to Neonatal Circulation: Normal Responses and Implications for Infants with Heart Disease,” Seminars in Perinatology (Philadelphia, Pennsylvania) 17(2):106-121), and
3. The immune system at birth is less highly developed than in toddler (see, for example, Adamczak, A. M., Werblinska, A., Jamka, M., and Walkowiak, J. 2024. “Maternal-Foetal/Infant Interactions—Gut Microbiota and Immune Health,” Online, MDPI [Multidisciplinary Digital Publishing Institute] Biomedicines (Basel, Switzerland) 12(3):490; Adeniyi-Ipadeola, G. O., Hankins, J. D., Kambal, A., Zeng. X. L., Patil, K., and 11 others 2024. “Infant and Adult Human Intestinal Enteroids are Morphologically and Functionally Distinct,” bioRxiv: The Preprint Server for Biology (Cold Spring Harbor, New York) 2023.05.19.541350; Shafique, M. A., Abbas, S. Q., Habiba, U., Mughal. A., Fadlalla Ahmad, T. K., and Munir Chaudhary, A. 2024. “Transfusion Strategies for [preterm] Neonates: Current Perspectives,” Online, Annals of Medicine and Surgery (London, England) 86(3):1550-1562; Kalbermatter, C., Fernandez Trigo, N., Christensen, S., and Ganal-Vonarburg, S. C. 2021. “Maternal Microbiota, Early Life Colonization, and Breast Milk Drive Immune Development in the Newborn,” Online, Frontiers in Immunology (Lausanne, Switzerland) 12:683022; Lele, M. 2021. “What to Know about Your Baby's Immune System Development Timeline,” at lilmixins.com/blogs/; National Health Service (U.K.) 2021. “How Long Do Babies Carry Their Mother's Immunity?,” at https://www.nhs.uk/childrens-health; Hillion, S., Arleevskaya, M. I., Blanco, P., Bordron. A., Brooks, W. H., and 4 others 2020. “The Innate Part of the Adaptive Immune System,” Clinical Reviews in Allergy and Immunology (Totowa, New Jersey) 58(2):151-154; Jain, N. 2020. “The Early Life Education of the Immune System: Moms, Microbes, and (Missed) Opportunities,” Online, Gut Microbes 12(1):1824564; Tsafaras, G. P., Ntontsi, P., and Xanthou, G. 2020. “Advantages and Limitations of the Neonatal Immune System,” Online, Frontiers in Pediatrics (Lausanne, Switzerland) 8:5; Children's Hospital of Philadelphia 2019. “Development of the Immune System,” at https://www.chop.edu; Yu. J. C., Khodadadi, H., Malik, A., Davidson, B., Salles, E. D. S. L., and 3 others 2018. “Innate Immunity of Neonates and Infants,” Online, Frontiers in Immunology (Lausanne, Switzerland) 9:1759; Yu, H. R., Huang, L. H., and Li, S. C. 2018. “Roles of MicroRNA in the Immature Immune System of Neonates,” Cancer Letters (Limerick, Ireland): 433:99-106; Georgountzou, A. and Papadopoulos, N. G. 2017. “Postnatal Innate Immune Development: From Birth to Adulthood,” Online, Frontiers in Immunology (Lausanne, Switzerland) 8:957; Kumar, S. K. and Bhat, B. V. 2016. “Distinct Mechanisms of the Newborn Innate Immunity,” Immunology Letters (Amsterdam, North Holland, Netherlands) 173:42-54; Simon, A. K., Hollander, G. A., and McMichael, A. 2015. “Evolution of the Immune System in Humans from Infancy to Old Age,” Online, Proceedings of the Royal Society B: Biological Sciences 282(1821):20143085; Swanson, J. R. and Sinkin, R. A. 2015. “Transition from Fetus to Newborn,” Pediatric Clinics of North. America (Philadelphia, Pennsylvania) 62(2):329-343; Basha, S., Surendran, N., and Pichichero, M. 2014. “Immune Responses in Neonates,” Online, Expert Review of Clinical Immunology (Oxford, England) 10(9):1171-1184; Futata, E. A., Fusaro, A. E., de Brito, C. A., and Sato, M. N. 2012. “The Neonatal Immune System: Immunomodulation of Infections in Early Life,” Expert Review of Anti-infective Therapy (Oxford, England) 10(3):289-298; Hillman, N. H., Kallapur, S. G., and Jobe, A. H. 2012. “Physiology of Transition from Intrauterine to Extrauterine Life,” Online, Clinics in Perinatology (Philadelphia, Pennsylvania) 39(4):769-783; Willems, F., Vollstedt, S., and Suter, M. 2009. “Phenotype and Function of Neonatal DC [Dendritic Cells],” Online, European Journal of Immunology Weinheim, Germany) 39(1):26-35; Levy, O. 2007. “Innate Immunity of the Newborn: Basic Mechanisms and Clinical Correlates,” Nature Reviews. Immunology (London, England) 7(5):379-390; Adkins, B., Leclerc. C., and Marshall-Clarke, S. 2004. “Neonatal Adaptive Immunity Comes of Age,” Nature Reviews. Immunology (London, England) 4(7):553-564; Crockett. M. 1995. “Physiology of the Neonatal Immune System,” Journal of Obstetric, Gynecologie, and Neonatal Nursing (New York, New York) 24(7):627-634; Durandy, A. 1992. “Development of the Immune System,” (in French with English abstract at Pubmed), Pathologie-biologie (Paris) 40(7):685-689).

Moreover, because the method of compound vascular bypass, or switched-organ, transplantation delineated herein leaves the graft heart intact, without the use cardioplegia, eliminates any interruption in perfusion, hence, ischemia-reperfusion injury, and can usually be performed under regional anesthesia, the procedure causes the least trauma, significantly increasing the odds for a favorable outcome.

That an implant system to automatically monitor and instantly respond to any out of range indicator of normal heart function plainly surpasses any alternative approach, and-the more so when a drug that should be isolated from untargeted tissue is, and when necessary, can be delivered in a higher dose. A fully five-fold if not greater increase in every dimension from neonate to adult cannot be complied with by using conventional tubing. This limitation is overcome through use of the highly expandable fabric addressed below in section 2b(2)(f), entitled Expandable Structure to Accommodate Growth and Self-adjust in Size in Vascular Segment Prostheses, Lines, and Confluence Conduits Placed in Children, which should accommodate growth for many years, and even in a larger adult, to the end of life.

This problem eliminated, the factor that limits the durability of a Rastelli procedure in a child, for example—the need to accommodate growth—pertains to electronic or mechanical automatic disorder response system components only after many years if ever. Currently, a heart transplanted into a prenate, neonate, or infant often requires replacement in 10 to 15 years during which the rate of growth is considerable, so that relatively little growth remains to adulthood. Accordingly, while the diversion jackets and lines can be left in place indefinitely, in a fetus or neonate, it is best to not allow these to remain in place for more than six months or well past the transplant procedure when the patient has fully recovered and is in better health.

Then the free transected ends of the arteries and veins closed off at the time of the transplant must be anastomosed. However, the design attributes enumerated above in section 2b(1)(a), entitled Sudden Compound Vascular Bypass, or Sudden Switched-organ Transplantation, should allow the lines and jackets placed at the outset of life to increase in step with growth for years. If not, to replace these can be accomplished after years endoscopically. Given that, in most instances, a replacement heart will itself eventually have to replaced, undersized components can be replaced at the same time, or if the original transplant continues to function well, the components can be changed in what is a relatively safe and nontraumatic procedure.

Intracorporeal ventricular assist devices to serve as a bridge until a new heart can be found are now made in the size required; however, replacement with a living heart, especially when implanted using the procedure delineated herein, especially when supported with implanted automatic disorder response system, is fundamentally superior to any alternative. The need for a total artificial heart for neonates has been recognized for more than three decades with inadequate or incomplete results to date (Koppert, E., Holfert, J. W., Dew. P. A., Juretich, J. T., Diegel, P. D., and Burns, G. L. 1991. “Preliminary in Vitro Evaluation of the First Neonatal Total Artificial Heart” American Society for Artificial Internal Organ Transactions 37(3):M127-M128; Koppert. E., Holfert, J. W., Dew. P. A., Pauley, J., Pantalos, G. M., Crump, K. R., and Burns, G. L. 1990. “A Total Artificial Heart for Neonates Allowing Bridging to Transplantation,” American Society for Artificial Internal Organ Transactions 36(3):M226-M230).

As is the case with adults, total artificial hearts for use in neonates and infants serve to sustain life as a bridge pending transplantation, but incapable of growth, are limited to short term support, and adaptation of an adult artificial heart to support a child is limited to use in the hospital. In adults, no total artificial heart to date has been approved for destination therapy but only as a bridge to a heart transplant (see, for example, Dipchand, A. I. 2018. “Current State of Pediatric Cardiac Transplantation,” Op cit., in section 2. Background of the Invention, a. General, (1). Congenital Defects of the Heart and Other Organs: The Importance of Prompt Correction; Fox. C., Chopski, S., Murad, N., Allaire, P., Mentzer, R. Jr., and 3 others 2018. “Hybrid Continuous-flow Total Artificial Heart,” Artificial Organs 42(5):500-509; Fukamachi, K., Karimov, J. H., Horvath, D. J., Sunagawa, G., Byram, N. A., Kuban, B. D., and Moazami, N. 2018. “Initial in Vitro Testing of a Paediatric Continuous-flow Total Artificial Heart,” Interactive Cardiovascular and Thoracic Surgery 26(6):897-901; Karimov, J. H., Horvath, D. J., Byram, N., Sunagawa, G., Kuban, B. D., and 3 others 2018. “Early in Vivo Experience with the Pediatric Continuous-flow Total Artificial Heart” Journal of Heart and Lung Transplantation (New York, New York) 37(8):1029-1034; Ziegler, L. A., Sainathan, S., Morell, V. O., and Sharma, M. S. 2018. “Total Artificial Heart as Rescue Therapy for Primary Graft Failure in an Infant” Annals of Thoracic Surgery 105(4):e151-e153; Chopski, S. G., Moskowitz, W. B., Stevens, R. M., and Throckmorton, A. L. 2017. “Mechanical Circulatory Support Devices for Pediatric Patients with Congenital Heart Disease,” Artificial Organs 41(1):E1-E14; Hetzer, R., Kaufmann, F., and Delmo Walter, E. M. 2016. “Paediatric Mechanical Circulatory Support with Berlin Heart EXCOR [pulsatile ventricular assist device]: Development and Outcome of a 23-year Experience,” European Journal of Cardiothoracic Surgery 50(2):203-210; Cook, J. A., Shah, K. B., Quader, M. A., Cooke, R. H., Kasirajan, V., and 4 others 2015. “The Total Artificial Heart” Journal of Thoracic Disease 7(12):2172-2180, also cited in sections to follow; Kirklin, J. K. 2015. “Advances in Mechanical Assist Devices and Artificial Hearts for Children,” Current Opinion in Pediatrics 27(5):597-603; Khan, S. and Jehangir, W. 2014. “Evolution of Artificial Hearts: An Overview and History,” Cardiology Research 5(5):121-125; Park, S. S., Sanders, D. B., Smith, B. P., Ryan, J., Plasencia, J., and 10 others 2014. “Total Artificial Heart in the Pediatric Patient with Biventricular Heart Failure,” Perfusion 29(1):82-88; Slepian, M. J., Alemu, Y., Girdhar, G., Soares, J. S., Smith, R. G., Einav, S., and Bluestein, D. 2013. “The Syncardia™ Total Artificial Heart: In Vivo, in Vitro, and Computational Modeling Studies,” Journal of Biomechanics 46(2):266-275; Maat. A. P., van Thiel, R. J., Dalinghaus, M., and Bogers, A. J. 2008. “Connecting the Centrimag Levitronix Pump to Berlin Heart Excor Cannulae; A New Approach to Bridge to Bridge,” Journal of Heart and Lung Transplantation (New York, New York) 27(1):112-115; Koppert, E., Pantalos, G. M., Tieleman, R., Swier, P., and Burns, G. L. 1991. “Cardiac Output Requirements and Maximum Dimensions for a Neonate Total Artificial Heart,” International Journal of Artificial Organs 14(11):707-715; Koppert, E., Holfert, J. W., Dew. P. A., Juretich, J. T., Diegel, P. D., and Burns, G. L. 1991. “Preliminary in Vitro Evaluation of the First Neonatal Total Artificial Heart,” American Society for Artificial Internal Organs Transactions 37(3):M127-M128; Koppert. E., Holfert, J. W., Dew. P. A., Pauley, J., Pantalos, G. M., Crump, K. R., and Burns, G. L. 1990. “A Total Artificial Heart for Neonates Allowing Bridging to Transplantation,” American Society for Artificial Internal Organs Transactions 36(3):M226-M230).

Today, the repair as opposed to the replacement of a serious heart malformity or its treatment by placement of an assist device should be considered an interim or bridging strategy in the same category as the placement of an assist device, or an interim replacement heart known to be impaired, or an artificial heart to keep the patient from dying before a replacement heart can be found (see, for example, references cited above under section 2a(3), entitled Remotely Procured Graft Organ Preservation; Miana, L. A., Silva, G. V. R. D., Caneo. L. F., Turquetto, A. L., Tanamati, C., and 7 others 2018. “Rational Use of Mechanical Circulatory Support as a Bridge to Pediatric and Congenital Heart Transplantation,” Brazilian Journal of Cardiovascular Surgery 33(3):242-249; Miller, J. R., Lancaster, T. S., Callahan, C., Abarbanell, A. M., and Eghtesady, P. 2018. “An Overview of Mechanical Circulatory Support in Single-ventricle Patients,” Translational Pediatrics 7(2):151-161; Theochari, C. A1., Michalopoulos, G., Oikonomou, E. K., Giannopoulos, S., Doulamis, I. P., Villela, M. A., and Kokkinidis, D. G. 2018. “Heart Transplantation Versus Left Ventricular Assist Devices as Destination Therapy or Bridge to Transplantation for 1-year Mortality: A Systematic Review and Meta-analysis,” Annals of Cardiothoracic Surgery (Hong Kong, China) 7(1):3-11; Griselli, M., Sinha. R., Jang, S., Perri, G., and Adachi, I. 2018. “Mechanical Circulatory Support for Single Ventricle Failure,” Online, Frontiers in Cardiovascular Medicine 5:115; Serfas. J. D., Patel, P. A., and Krasuski, R. A. 2018, Op cit., above under section 2a(3), entitled Remote Graft Organ Preservation; Steiner, J. M., Krieger, E. V., Stout, K. K., Stempien-Otero, A., Mahr. C., Mokadam, N. A., and Hermsen, J. L. 2017. “Durable Mechanical Circulatory Support in Teenagers and Adults with Congenital Heart Disease: A Systematic Review,” International Journal of Cardiology 245:135-140; Wehman, B., Stafford, K. A., Bittle, G. J., Kon, Z. N., Evans, C. F., and 4 others 2016. “Modern Outcomes of Mechanical Circulatory Support as a Bridge to Pediatric Heart Transplantation,” Annals of Thoracic Surgery 101(6):2321-2327; Clerkin, K. J., Thomas, S. S., Haythe, J., Schulze, P. C., Farr. M., and 5 others 2015. Op cit. above under section 2a(3), entitled Remotely Procured Graft Organ Preservation); Maxwell, B. G., Wong, J. K., Sheikh, A. Y., Lee, P. H., and Lobato. R. L. 2014. Heart Transplantation with or without Prior Mechanical Circulatory Support in Adults with Congenital Heart Disease,” European Journal of Cardiothoracic Surgery 45(5):842-846; De Rita, F., Hasan, A., Haynes, S., Crossland, D., Kirk, R., and 3 others 2014. “Mechanical Cardiac Support in Children with Congenital Heart Disease with Intention to Bridge to Heart Transplantation,” European Journal of Cardiothoracic Surgery 46(4):656-662; Botha, P., Solana, R., Cassidy, J., Parry, G., Kirk, R., Hasan, A., and Griselli. M. 2013. “The Impact of Mechanical Circulatory Support on Outcomes in Paediatric Heart Transplantation,” European Journal of Cardiothoracic Surgery 44(5):836-840; Rossano, J. W., Woods, R. K., Berger, S., Gaynor, J. W., Ghanayem, N., and 12 others 2013. “Mechanical Support as Failure Intervention in Patients with Cavopulmonary Shunts (MFICS): Rationale and Aims of a New Registry of Mechanical Circulatory Support in Single Ventricle Patients,” Congenital Heart Disease 8(3):182-186; Khan, M. S., Mery, C. M., Zafar, F., Adachi, I., Heinle, J. S., and 3 others 2012. Op cit. above under section 2a(3), entitled Remotely Procured Graft Organ Preservation).

Primarily because heart transplantation using switch technique is extracardiac, involving no incision into the heart into both native and donor hearts, it is better tolerated and suited to prenatal and neonatal heart transplantation. Surgical trauma to both native and donor hearts is true of both conventional orthotopic and conventional heterotopic heart transplantation, and this imposition of injury at the outset reduces the odds for a favorable outcome, which switch transplantation eliminates. The implantation of an automatic monitoring and response system is of much advantage in conjunction with any surgical procedure, but is especially so with respect to the heart and transplantation.

The umbilical cord connected throughout, cardiopulmonary bypass, aortic cross-clamping, and general anesthesia are unused as in using switch technique in postpartum and adult heart transplantation. These complexities and the risk they carry omitted, the operators are freed to deal with the tiny size of the vessels, hence, the valve-jackets used. The hearts and other solid organs of miscarried and stillborn fetuses may have been dead well before detection, and the cause may have been cardiomyopathy. Anencephalic fetuses and those with other unsurvivable genetic defects usually have a functioning brainstem making them legally alive and the taking of their organs unlawful. Moreover, upon expiration, genetic defects often pleiotropic, hence, potentially multiple in different organ systems, the suitability for transplantation of organs were these taken from such babies having been somatically supported beginning in anticipation of death must be considered suspect.

The recipient in switch transplantation not placed on cardiopulmonary support, dissection for cannulation is eliminated. Exceptionally, the recipient of a heart transplant can survive postoperatively for decades (Yousefzai. R., Trivedi, S., Jain, R., Cheema, O. M., Crouch, J. D., Thohan, V., and Khandheria, B. K. 2015. “Expecting the Unexpected: Right Atrial Mass in a Transplant Patient,” European Society of Cardiology Heart Failure 2(4):164-167), but generally an additional dozen years is a more reasonable expectation (see, for example, Alraies, M. C. and Eckman, P. 2014. “Adult Heart Transplant: Indications and Outcomes,” Journal of Thoracic Disease 6(8):1120-1128; Kinkhabwala, M. P. and Mancini, D. 2013. “Patient Selection for Cardiac Transplant in 2012,” Expert Review of Cardiovascular Therapy 11(2):179-191; Chan, W. X. and Ross, H. 2012. “Who Needs a Transplant and When?,” Current Opinion in Organ Transplantation 17(5):531-539), in this case, recipient off-pump, or beating-heart transplantation, have numerous applications in creating shunts and bypasses which involve relatively little trauma to place, and allow urinary diversion from the ureter to the bladder or a paracorporeal collection bag, for example.

For cardiovascular procedures on fetuses, neonates, and infants, the tissue expansion offered by the intrinsic mechanotransduction pathways of autologous patch tissue, which serves to generate the development of new contiguous tissue, accommodates growth, averting the need for revision (see, for example, Talwar, S., Choudhary, S. K., Anita Saxena, Kothari, S. S., Juneja, R., and Airan, B. 2008. “Unidirectional Valved Patches for Closure of Septal Defects in Patients with Severe Pulmonary Hypertension,” Annals of Pediatric Cardiology 1(2):114-119; Konstantinov, I. E., Alexi-Meskishvili, V. V., Williams, W. G., Freedom, R. M., and Van Praagh, R. 2004. “Atrial Switch Operation: Past, Present, and Future,” Annals of Thoracic Surgery 77(6):2250-2258) is compensated for by two factors provided by the jackets and one factor contributed by the lines described herein.

The first is the foam jacket lining, which compresses as the substrate vessel expands in diameter. The other is that the restorative force of the jacket spring hinges is set to allow the jacket to progressively open once the compressive limit of the foam lining has been exceeded. The third is that the lines, both mainlines and accessory channels, or sidelines, consist of coiled plastic springs loosely wrapped about by thin but strong plastic film capable of great change in length with no collateral alteration in diameter, which factor also allows the lines to be long enough during either the sudden or metered compound vascular bypass, or sudden switched-organ transplantation procedure without presenting undue bulk once the new organ is implanted.

Instrumental in increasing the supply of available replacement hearts for use in the very young, tissue expansion may precede the placement of an oversized heart with a higher ventricular absolute stroke volume and the expectation that the new heart, continuously monitored and supported by the implanted automatic disorder control system will adjust to the lighter load. It is seen most clearly in the balloon stretching of the skin practiced by plastic surgeons to obtain additional tissue to extend over a wound or where tissue has been lost or resected (see, for example, Zöllner, A. M., Buganza Tepole, A., Gosain, A. K., and Kuhl, E. 2012. “Growing Skin: Tissue Expansion in Pediatric Forehead Reconstruction,” Biomechanics and Modeling in Mechanobiology 11(6):855-867; Zollner, A. M., Buganza Tepole, A., and Kuhl, E. 2012. “On the Biomechanics and Mechanobiology of Growing Skin,” Journal of Theoretical Biology 297:166-175; Neumann, C. G. 1946, republished 1957. “The Expansion of an Area of Skin by Progressive Distention of a Subcutaneous Balloon: Use of the Method for Securing Skin for Subtotal Reconstruction of the Ear,” Plastic and Reconstructive Surgery 19(2):124-130).

The type procedure to be described should reduce the need for retransplantation and extend survival times. The same procedure can be used to place an artificial heart, usually done to serve as a “bridge” or intermediate measure until a suitable donor heart becomes available. The same will apply once tissue engineered replacement hearts become available, although that will take a long time. Side-entry diversion jackets are side-entry jackets modified to divert excurrent, or when inverted, incurrent flow through a bodily ductus or conduit. A conventional side-entry jacket will redirect a portion of the flow through it according to its internal diameter, but this proportion is fixed.

Today, the repair of a serious heart malformity should be considered an interim or bridging strategy in the same category as the placement of an assist device, a replacement heart known to be impaired, or an artificial heart to keep the patient from dying before a replacement heart can be found (see, for example, references cited above under section 2a(3), entitled Remotely Procured Graft Organ Preservation; Miana, L. A., Silva, G. V. R. D., Caneo, L. F., Turquetto, A. L., Tanamati, C., and 7 others 2018. “Rational Use of Mechanical Circulatory Support as a Bridge to Pediatric and Congenital Heart Transplantation,” Brazilian Journal of Cardiovascular Surgery 33(3):242-249; Miller, J. R., Lancaster, T. S., Callahan, C., Abarbanell, A. M., and Eghtesady, P. 2018. “An Overview of Mechanical Circulatory Support in Single-ventricle Patients,” Translational Pediatrics 7(2):151-161; Theochari. C. A1., Michalopoulos, G., Oikonomou, E. K., Giannopoulos. S., Doulamis, I. P., Villela, M. A., and Kokkinidis, D. G. 2018. “Heart Transplantation Versus Left Ventricular Assist Devices as Destination Therapy or Bridge to Transplantation for 1-year Mortality: A Systematic Review and Meta-analysis,” Annals of Cardiothoracic Surgery (Hong Kong, China) 7(1):3-11; Griselli, M., Sinha, R., Jang. S., Perri, G., and Adachi, I. 2018. “Mechanical Circulatory Support for Single Ventricle Failure,” Online, Frontiers in Cardiovascular Medicine 5:115; Serfas, J. D., Patel, P. A., and Krasuski, R. A. 2018, Op cit., above under section 2a(3), entitled Remotely Procured Graft Organ Preservation; Carvajal, J. A., Ferrer, F. A., Araya, F. I., Delpiano, A. M. 2017. “Normal Amino-terminal Pro-brain Natriuretic Peptide (NT-proBNP) Values in Amniotic Fluid,” Clinical Biochemistry 50(1-2):23-26; Leufgen, C., Gembruch, U., Stoffel-Wagner, B., Fimmers. R., and Merz, W. M. 2017. “N-terminal Pro-B-type Natriuretic Peptide in Amniotic Fluid of Fetuses with Known or Suspected Cardiac Load,” Online, PLoS [Public Library of Science] One 12(5):e0177253; Steiner, J. M., Krieger, E. V., Stout, K. K., Stempien-Otero, A., Mahr. C., Mokadam, N. A., and Hermsen, J. L. 2017. “Durable Mechanical Circulatory Support in Teenagers and Adults with Congenital Heart Disease: A Systematic Review,” International Journal of Cardiology 245:135-140; Wehman, B., Stafford, K. A., Bittle, G. J., Kon, Z. N., Evans, C. F., and 4 others 2016. “Modern Outcomes of Mechanical Circulatory Support as a Bridge to Pediatric Heart Transplantation,” Annals of Thoracic Surgery 101(6):2321-2327; Clerkin, K. J., Thomas, S. S., Haythe, J., Schulze, P. C., Farr. M., and 5 others 2015, Op cit. above under section 2a(3), entitled Remotely Procured Graft Organ Preservation); Maxwell, B. G., Wong, J. K., Sheikh, A. Y., Lee, P. H., and Lobato, R. L. 2014. Heart Transplantation with or without Prior Mechanical Circulatory Support in Adults with Congenital Heart Disease,” European Journal of Cardiothoracic Surgery 45(5):842-846; De Rita, F., Hasan, A., Haynes, S., Crossland, D., Kirk, R., and 3 others 2014. “Mechanical Cardiac Support in Children with Congenital Heart Disease with Intention to Bridge to Heart Transplantation,” European Journal of Cardiothoracic Surgery 46(4):656-662; Botha. P., Solana. R., Cassidy, J., Parry, G., Kirk, R., Hasan, A., and Griselli, M. 2013. “The Impact of Mechanical Circulatory Support on Outcomes in Paediatric Heart Transplantation,” European Journal of Cardiothoracic Surgery 44(5):836-840; Rossano, J. W., Woods, R. K., Berger, S., Gaynor, J. W., Ghanayem, N., and 12 others 2013. “Mechanical Support as Failure Intervention in Patients with Cavopulmonary Shunts (MFICS): Rationale and Aims of a New Registry of Mechanical Circulatory Support in Single Ventricle Patients,” Congenital Heart Disease 8(3):182-186; Khan, M. S., Mery, C. M., Zafar, F., Adachi, I., Heinle, J. S., and 3 others 2012, Op cit. above under section 2a(3), entitled Remotely Procured Graft Organ Preservation).

Considerably simplified, a heart, for example, may be intrinsically affected by a genetic rather than a developmental structural disorder, that is, primarily diseased so that transplanting it could transfer the disease along with the heart, or primarily diseased where the direct pipeline targeting of medication to the coronary arteries would control the problem, expanding the zone of acceptability for substandard organs additionally supported medically by the implanted automatic response system.

Or the heart of another organ may be secondarily diseased where, for example, a metabolic disorder substantially originating in the liver or a systemic infection when treated would cease to exert any effect once the heart was placed in another body (Sahlin, E., Green, A., Gustavsson, P., Lieden, A., Nordenskjold, M., and 5 others 2019. “Identification of Putative Pathogenic Single Nucleotide Variants (SNVs) in Genes Associated with Heart Disease in 290 Cases of Stillbirth,” Online, PLoS [Public Library of Science] One 14(1):e0210017; Stone, W. L. and Adil, A. 2019. “Glycogen Storage Disease,” Online, Treasure Island, Florida: StatPearls Publishing Co.; Kishnani, P. S., Goldstein, J., Austin, S. L., Am, P., Bachrach, B., and 10 others 2019. “Diagnosis and Management of Glycogen Storage Diseases Type VI and IX: A Clinical Practice Resource of the American College of Medical Genetics and Genomics (ACMG),” Genetics in Medicine 21(4):772-789; Munroe, P. B., Addison, S., Abrams, D. J., Sebire, N. J., Cartwright, J., and 15 others 2018. “Postmortem Genetic Testing for Cardiac Ion Channelopathies in Stillbirths,” Circulation. Genomic and Precision Medicine 11(1):e001817; Li, G. L., Saguner, A. M., and Fontaine, G. H. 2018. “Naxos Disease: From the Origin to Today,” Orphanet Journal of Rare Diseases 13(1):74; Kanungo, S., Wells, K., Tribett, T., and El-Gharbawy, A. 2018. “Glycogen Metabolism and Glycogen Storage Disorders,” Annals of Translational Medicine 6(24):474; Connell, P. S., Jeewa, A., Kearney, D. L., Tunuguntla, H., Denfield, S. W., Allen, H. D., and Landstrom, A. P. 2018. “A 14-year-old in Heart Failure with Multiple Cardiomyopathy Variants Illustrates a Role for Signal-to-noise Analysis in Gene Test Re-interpretation,” Clinical Case Reports 7(1):211-217; Lee, T. M., Hsu, D. T., Kantor, P., Towbin, J. A., Ware, S. M., and 14 others 2017. “Pediatric Cardiomyopathies,” Circulation Research 121(7):855-873; Towbin, J. A. and Jefferies, J. L. 2017. “Cardiomyopathies Due to Left Ventricular Noncompaction, Mitochondrial and Storage Diseases, and Inborn Errors of Metabolism,” Circulation Research 121(7):838-854; Mckenna, W. J., Maron, B. J., and Thiene, G. 2017. “Classification, Epidemiology, and Global Burden of Cardiomyopathies,” Circulation Research 121(7):722-730; Braunwald, E. 2017. “Cardiomyopathies: An Overview,” Circulation Research 121(7):711-721; Sisakian, H. 2014. “Cardiomyopathies: Evolution of Pathogenesis Concepts and Potential for New Therapies,” World Journal of Cardiology 6(6):478-494; Byers, S. L. and Ficicioglu, C. 2014. “Infant with Cardiomyopathy: When to Suspect Inborn Errors of Metabolism?,” World Journal of Cardiology 6(11):1149-1155; Boers, S., Visser, G., Smit, P. G., and Fuchs, S. A. 2014. “Liver Transplantation in Glycogen Storage Disease Type I,” Orphanet Journal of Rare Diseases 9:47; Lipshultz, S. E., Cochran, T. R., Briston, D. A., Brown, S. R., Sambatakos, P. J., and 12 others 2013. “Pediatric Cardiomyopathies: Causes, Epidemiology, Clinical Course, Preventive Strategies and Therapies,” Future Cardiology 9(6):817-848; Wicks, E. C. and Elliott, P. M. 2012. “Genetics and Metabolic Cardiomyopathies,” Her: 37(6):598-610; Hayes, W. L., Dimond, E. G., and Crockett, J. E. 1959. “Idiopathic Cardiomegaly with Extreme Cardiac Enlargement: Hypoplastic Aorta as Primary of Secondary Factor?,” American Journal of Cardiology 4:528-534), or primarily diseased where the direct pipeline targeting of medication to the coronary arteries would control the problem, expanding the zone of acceptability for substandard organs, or secondarily diseased where, for example, a metabolic disorder substantially originating in the liver or a systemic infection when treated would cease to exert any effect once the heart was placed in another body.

In this circumstance, hearts must be procured from later term fetuses and neonates identified as irreversibly and fatally ill without significant heart involvement or malformity placed on somatic support prior to death (see, for example, Bale, J. R., Stoll, B. J., Lucas, A. O. (eds.) 2003. Improving Birth Outcomes: Meeting the Challenge in the Developing World. Washington, D.C.: National Academies Press) and those healthy but slated for intentional abortion, where death having been scheduled notwithstanding, these babies are alive and can be placed on somatic support prior to being killed, where, however, these will become fewer as more states institute ‘beating heart’ laws.

Also alive are babies with fatal diseases which result in death over a longer term, such as the GM2 gangliosidoses Sandhoff disease, infantile onset thereof not resulting in organomegaly (see Tavasoli, et al. cited below), Tay-Sachs disease, AB variant GM2-gangliosidosis, and severe glycogen storage disease type II or Pompe disease, albeit often (Kanungo, S., Wells, K., Tribett, T., and El-Gharbawy, A. 2018. “Glycogen Metabolism and Glycogen Storage Disorders,” Annals of Translational Medicine 6(24):474) prone to cardiomegaly, or hypertrophic cardiomyopathy, however, in such cases, the institution of life support prior to brain death is possible. Placed in an infant without the glycogen storage disease, a cardiomegalic heart respond to treatment to the extent that it attain normal or acceptable performance.

Glycogen storage disease results in cardiomyopathy, so that a heart taken from such a donor would require surgical repair while still in the donor under somatic support first, then intensive medical support over and above that needed to counteract rejection once transplanted. The use of such a doubly impaired heart should be discounted except in the dire circumstances where mechanical aids are decline in favor of a real heart for use as a bridge pending receipt of a better one.

Children with infantile onset glycogen storage disease tend to die at 4.7 to 18.7 months of age, so that unless death occurred early in this range, there is the additional drawback in that the graft organ will be too large. Both contingencies—the availability of a heart too large or too small bode complications, one too large within limits eventually adapting to the lesser load but only after an initial critical period of hypertension with the possibility seizures and coma, and one too small incapable of producing an adequate blood pressure.

By allowing the detection and immediate delivery of drugs to a heterotopic heart piggybacked on a congenitally inadequate heart or piggybacked to the donor heart before the circulation of the recipient is switched to the new double heart, ductus side-entry jackets may allow not only overcoming a defective native heart but maintaining it under constant vigilance and treatment, and by making possible the diversion of flow from a heart taken from an older child and too large for an infant, then continuously monitoring the blood pressure and adjusting it as necessary through servovalve-jackets, the way is opened to consider a replacement heart that would previously have to be discounted.

Clinical experience too slight to draw any hard conclusions, complications following the implantation of a heart taken from a donor weighing almost four times as much as the recipient have been treated through the initial hypertensive period with acceptable results, indicating that an even greater disparity in body mass would be necessary to induce a ‘big-heart syndrome’ that failed to subside within a week and remained refractory to remediation (Shin, H. J., Jhang, W. K., Park, J. J., Yun, T. J., Kim, Y. H., and 3 others 2011, Op cit. above under I. Background of the Invention. (a). General; Chou, N. K., Chang, C. H., Chi, N. H., Chang, C. I., Chen, Y. S., and 9 others 2006. “Single-center Experience of Pediatric Heart transplantation in Taiwan,” Transplantation Proceedings 38(7):2130-2131).

Within an undetermined upper limit for oversize, a cardiac allograft will initially overdrive the circulatory system to hypertension, risking coma and convulsions. At least to an undetermined upper limit of oversize, during which antihypertensives are used to reduce the blood pressure (Shin, H. J., Jhang, W. K., Park, J. J., Yun, T. J., Kim, Y. H., and 3 others 2011, Op cit. above under section 2a, entitled Background of the Invention. General; Schumacher, K. R. and Gajarski, R. J. 2011. “Postoperative Care of the Transplanted Patient,” Current Cardiology Reviews 7(2):110-122; Vega, E., Schroder, J., and Nicoara, A. 2017. “Postoperative Management of Heart Transplantation Patients,” Best Practice and Research. Clinical Anesthesiology 31(2):201-213), the size factor appears tractable.

Larger still, a heart taken from a young adult, for example, might persist in producing adverse sequelae indefinitely, which may or may not be suppressed with medication. If neither a better heart nor mechanical support is available, and the adverse consequences do not subside and/or are not controlled by antihypertensives then the use of continuously controlled flow diversion valves to reduce the pressure of ejection through the ascending aorta by apportioned dispersal of a portion of the outflow to different larger vessels may allow the placement of what would otherwise be too large heart, preferably without the need for nifedipine to avert the convulsions and coma associated with “big-heart syndrome.”

Further to expand the zone of hearts acceptable for transplantation, the lessons learned from placing a previously more exercised heart in an infant and the means for suppressing the consequences of hypertension pending self-adjustment to the less resistive load can be augmented through the implantion of blood pressure sensors and an automatically responding system to directly pipeline target anti-inflammatoiremmunosuppressants, and antimicrobials through drug conveying accessory channels of the pressure diversion servovalve to the blood supply of the graft organ with other diversion servovalves on the renal, inferior phrenic, and middle suprarenal arteries.

These other entry points are preferably accessed with the aid of contrast through minimal incisions, or if through the use of a trocar, then an optical trocar (see, for example, Ahmad, G., Baker, J., Finnerty, J., Phillips, K., and Watson, A. 2019. “Laparoscopic Entry Techniques,” Cochrane Database of Systematic Reviews 1: CD006583; Kaistha, S., Kumar, A., Gangavatiker, R., Br, S., and Sisodiya, N. 2019. “Laparoscopic Access: Direct Trocar Insertion Versus Open Technique,” Journal of Laparoendoscopic and Advanced Surgical Techniques. Part.A. 29(4):489-494; Chaturvedi, A., Shukair, S. A., Le Rolland, P., Vijayvergia, M., Gunn, J. W., and Subramanian, H. 2018. “Blood Vessel Detection, Localization and Estimation Using a Smart Laparoscopic Grasper: A Monte Carlo Study,” Biomedical Optics Express 9(5):2027-2040; Chaturvedi, A., Shukair, S. A., Vijayvergia, M., Le Rolland, P., Gunn, J. W., and Subramanian, H. 2018. “Intraoperative Blood Vessel Detection and Quantification: A Monte Carlo Study,” Journal of Biomedical Optics 23(6):1-9; Alkatout, I. 2017. “Complications of Laparoscopy in Connection with Entry Techniques,” Journal of Gynecologic Surgery 33(3):81-91; Alkatout, I., Mettler, L., Maass, N., Noe, G. K., and Elessawy, M. 2015. “Abdominal Anatomy in the Context of Port Placement and Trocars,” Journal of the Turkish German Gynecological Association 16(4):241-251; Vilos, G. A., Temamian, A., Dempster, J., and Laberge, P. Y. with 11 collaborators 2007. “Laparoscopic Entry: A Review of Techniques, Technologies, and Complications,” Journal of Obstetrics and Gynaecology Canada 29(5):433-447; String, A., Berber, E., Foroutani, A., Macho, J. R., Pearl, J. M., and Siperstein, A. E. 2001. “Use of the Optical Access Trocar for Safe and Rapid Entry in Various Laparoscopic Procedures,” Surgical Endoscopy 15(6):570-573).

Finding a heart of suitable size and ventricular absolute stroke volume is precisely the problem faced in finding a replacement heart for a neonate or an infant. Tissue expansion and other plastic surgical techniques allowing the emplacement of an overly large heart, the problem is primarily medical. A means for allowing the use of a significantly larger heart that much reduced the risks involved would significantly expand the zone of acceptable donor hearts to include both those which pose a temporary problem until spontaneously adapted to the smaller body and those large enough as to pose a chronic risk of recurrent coma or convulsions.

Such eventualities are addressed in the literature (see, for example, Cho, H. J., Seo, D. M., Jhang, W. K., Park, C. S., and Kim, Y. H. 2009. “Transplantation of an Extremely Oversized Heart after Prolonged Extracorporeal Membrane Oxygenation Assistance in a 3-month-old Infant with Congenital Heart Disease,” Korean Journal of Thoracic and Cardiovascular Surgery 42(5) 630-634; Razzouk, A. J., Johnston, J. K., Larsen, R. L., Chinnock, R. E., Fitts, J. A., and Bailey, L. L. 2005. “Effect of Oversizing Cardiac Allografts on Survival in Pediatric Patients with Congenital Heart Disease,” Journal of Heart and Lung Transplantation (New York, New York) 24(2):195-199; McGiffin, D. C., Kirklin, J. K., and Pearce, F. B. 1997. “Pediatric Cardiac Transplantation,” Advances in Cardiac Surgery 9:149-176; Bernstein, D. 1993. “Update on Cardiac Transplantation in Infants and Children,” Critical Care Medicine 21(9 Supplement): S354-S355; Reichart, B. 1992. “Size Matching in Heart Transplantation,” Journal of Heart and Lung Transplantation (New York, New York) 11(4 Part 2):S199-S202).

Otherwise, the adequacy of a heart too small, one taken from the opposite sex, or placed in an obese donor is largely established (see, for example, Bergenfeldt, H., Stehlik, J., Hoglund, P., Andersson, B., and Nilsson, J. 2017. “Donor-recipient Size Matching and Mortality in Heart Transplantation: Influence of Body Mass Index and Gender,” Journal of Heart and Lung Transplantation (New York, New York) 36(9):940-947; Jalowiec, A., Grady, K. L., and White-Williams, C. 2017. “Mortality, Rehospitalization, and Post-transplant Complications in Gender-mismatched Heart Transplant Recipients,” Heart and Lung: The Journal of Critical Care 46(4):265-272; Martinez-Selles, M., Almenar, L., Paniagua-Martin, M. J., Segovia, J., Delgado, J. F., and 12 others 2015. “Donor/Recipient Sex Mismatch and Survival after Heart Transplantation: Only an Issue in Male Recipients? An Analysis of the Spanish Heart Transplantation Registry,” Transplant International 28(3):305-313; Reed, R. M., Netzer, G., Hunsicker, L., Mitchell, B. D., Rajagopal, K., Scharf, S., and Eberlein, M. 2014. “Cardiac Size and Sex-matching in Heart Transplantation: Size Matters in Matters of Sex and the Heart,” Journal of the American College of Cardiology. Heart Failure 2(1):73-83; Jalowiec, A., Grady, K. L., and White-Williams, C. 2012. “First-year Clinical Outcomes in Gender-mismatched Heart Transplant Recipients,” Journal of Cardiovascular Nursing 27(6):519-527; Fiorelli, A. I., Coelho, G. H., Lima, J. L., Lourenço, D. D., Gutierres, P., and 4 others 2009. “Massive Degeneration and Atrophy of the Native Heart after Heterotopic Transplantation: A Case Report,” Transplantation Proceedings 41(3):965-966, also cited below in section 2b(1)(b)(b5), entitled Heterotopic Double Heart Compound Vascular Bypass, or Sudden Switched-organ Transplantation [and so on]; Smith, R. D. Jr., Radovancevic, B., Gregoric, I. D., Cohn, W. E., and Frazier, O. H. 2006. “Orthotopic Retransplantation in Heterotopic Transplant Recipients: 3 Case Reports,” Journal of Heart and Lung Transplantation (New York, New York) 25(2):245-247; Boffini, M., Ragni, T., Pellegrini, C., Goggi, C., D'Armini, A. M., Rinaldi, M., and Viganò, M. 2004. “Heterotopic Heart Transplantation: A Single-centre Experience,” Transplantation Proceedings 6(3):638-640; Kadner, A., Chen, R. H., and Adams, D. H. 2000. “Heterotopic Heart Transplantation: Experimental Development and Clinical Experience,” European Journal of Cardiothoracic Surgery 17(4):474-481; Kawaguchi, A. T., Gandjbakhch, I., Desruennes, M., Pavie, A., Bors, V., and 5 others named 1995. “Orthotopic vs Heterotopic Heart Transplantation in Donor/Recipient Size Mismatch,” Transplantation Proceedings 27(1):1277-1281; additional references cited above under section 2b(1) entitled Intercorporeal Diversion of Blood Flow. (a). Sudden Compound vascular bypass, or sudden switched-organ Transplantation).

Lipid storage disease does affect the heart but secondarily, by affecting the nervous system, but does not affect it to the extent that if kept functioning under somatic support, then removed from the diseased milieu, placed in a metabolically normal recipient, and aggressively treated, the heart could not be used (Cachon-Gonzalez, M. B., Wang, S. Z., Ziegler, R., Cheng, S. H., and Cox, T. M. 2014. “Reversibility of Neuropathology in Tay-Sachs-related Diseases,” Human Molecular Genetics 23(3):730-748; Rodriguez-Torres, R., Schneck, L., and Kleinberg, W. 1971. “Electrocardiogramd Biochemical Abnormalities in Tay-Sachs Disease,” Bulletin of the New York Academy of Medicine 47(7):717-730).

Metabolic conditions can seriously affect the heart, which may, however, be salvageable once removed from the diseased donor and placed in the nondiseased recipient (see, for example, Kanungo, S., Wells, K., Tribett, T., and El-Gharbawy, A. 2018, Op cit. above in this section; Tavasoli, A. R., Parvaneh, N., Ashrafi, M. R., Rezaei, Z.1, Zschocke, J., and Rostami, P. 2018. “Clinical Presentation and Outcome in Infantile Sandhoff Disease: A Case Series of 25 Patients from Iranian Neurometabolic Bioregistry with Five Novel Mutations,” Orphanet Journal of Rare Diseases 13(1):130; Martínez, M., Romero, M. G., Guereta, L. G., Cabrera, M., Regojo, R. M., and 3 others 2017. “Infantile-onset Pompe Disease with Neonatal Debut: A Case Report and Literature Review,” Medicine (Baltimore, Maryland) 96(51):e9186; Sheth, J., Datar, C., Mistri. M., Bhavsar, R., Sheth, F., and Shah, K. 2016. “GM2 Gangliosidosis AB Variant: Novel Mutation from India—A Case Report with a Review,” Online, BioMed Central Pediatrics 16:88; Chien, Y. H., Hwu, W. L., and Lee, N. C. 2013. “Pompe Disease: Early Diagnosis and Early Treatment Make a Difference,” Pediatrics and Neonatology 54(4):219-227; Sakpichaisakul. K., Taeranawich, P., Nitiapinyasakul. A., and Sirisopikun, T. 2010. “Identification of Sandhoff Disease in a Thai Family: Clinical and Biochemical Characterization,” Journal of the Medical Association of Thailand 93(9):1088-1092; Dixon, C. A., Anderson, J. B., Ruddy, R. M., and Cripe. L. H. 2010. “Infantile-onset Pompe disease: A Diagnosis Not to Miss,” Pediatric Emergency Care 26(4):293-295; Hamdan, M. A., Almalik, M. H., and Mirghani, H. M. 2008. “Early Administration of Enzyme Replacement Therapy for Pompe Disease: Short-term Follow-up Results,” Journal of Inherited Metabolic Disease 31 Supplement 2: S431-S436; Venugopalan, P. and Joshi. S. N. 2002. “Cardiac Involvement in Infantile Sandhoff Disease,” Journal of Paediatrics and Child Health 38(1):98-100; Rodriguez-Torres, R., Schneck, L., and Kleinberg. W. 1971, Op cit. above in this section).

It having been determined that a somewhat oversized heart will adapt to the lower ejection pressure required by a smaller child, the baby pending death with prosthetic disorder response system implanted may be life extended by means such as a bone marrow transplantation. These diseases affecting the central nervous system, primarily the brain, and other organs secondarily if at all, sustainment thus must be balanced with the extension in time allowed for the disease to impair the organs desired when applicable.

As to therapeutic measures based upon animal models, many things work in murids which do not in humans (see, for example, Woodley, E., Osmon, K. J. L., Thompson, P., Richmond, C., Chen, Z., Gray, S. J., and Walia, J. S. 2019. “Efficacy of a Bicistronic Vector for Correction of Sandhoff Disease in a Mouse Model,” Molecular Therapy. Methods and Clinical Development 12:47-57; Stepien, K. M., Lum, S. H., Wraith, J. E., Hendriksz, C. J., Church, H. J., and 5 others 2018. “Haematopoietic Stem Cell Transplantation Arrests the Progression of Neurodegenerative Disease in Late-onset Tay-Sachs Disease,” Journal of Inherited Metabolic Disorders Reports 41:17-23; Solovyeva. V. V., Shaimardanova, A. A., Chulpanova, D. S., Kitaeva, K. V., Chakrabarti. L., and Rizvanov, A. A. 2018. “New Approaches to Tay-Sachs Disease Therapy,” Online, Frontiers in Physiology 9:1663; Nestrasil, I., Ahmed, A., Utz, J. M., Rudser, K., Whitley, C. B., and James-Utz, J. R. 2018. “Distinct Progression Patterns of Brain Disease in Infantile and Juvenile Gangliosidoses: Volumetric Quantitative MRI Study,” Molecular Genetics and Metabolism 123(2):97-104; Cachon-Gonzalez, M. B., Zaccariotto, E., and Cox, T. M. 2018. “Genetics and Therapies for GM2 Gangliosidosis,” Current Gene Therapy 18(2):68-89; Lawson, C. A. and Martin, D. R. 2016. “Animal Models of GM2 Gangliosidosis: Utility and Limitations,” Application of Clinical Genetics 9:111-120; Walia. J. S., Altaleb. N., Bello, A., Kruck, C., LaFave, M. C., and 7 others 2015. “Long-term Correction of Sandhoff Disease following Intravenous Delivery of rAAV9 to Mouse Neonates,” Molecular Therapy 23(3):414-422; Tsuji, D. 2013. “Molecular Pathogenesis and Therapeutic Approach of GM2 Gangliosidosis,” (in Japanese with English abstract at Pubmed), Yakugaku Zasshi [pharmaceutical science journal] (Journal of the Pharmaceutical Society of Japan) 133(2):269-274; Shihabuddin, L. S. and Cheng, S. H. 2011. “Neural Stem Cell Transplantation as a Therapeutic Approach for Treating Lysosomal Storage Diseases,” Neurotherapeutics 8(4):659-667; Maegawa, G. H., Stockley, T., Tropak, M., Banwell, B., Blaser, S., and 4 others 2006. “The Natural History of Juvenile or Subacute GM2 Gangliosidosis: 21 New Cases and Literature Review of 134 Previously Reported,” Pediatrics 118(5):e1550-e1562).

Where a prenate may have been parentally chimerized, neonates and infants such as those with perinatal hemorrhagic shock, a need for recurrent correction of anemia of prematurity, and/or with a functional single ventricle heart, for example, not repairable to provide normal circulation and therefore requiring a transplant, may additionally present high levels of preexisting panel reactive alloantibodies due to previous blood transfusions and/or the implantation of allogenic stem cells or other tissues that might render more difficult the effort to induce greater immune tolerance with respect to the prospective donor heart.

The prenate or neonate with undeveloped immune system and therefore a greater susceptibility to microchimerization through transfusion can be compared to fetomaternal microchimerism and patients severely immunocompromised due to trauma (see, for example, Shree, R., Harrington, W. E., Kanaan, S. B., Forsyth, A., Cousin, E., and 3 others 2019. “Fetal Microchimerism by Mode of Delivery: A Prospective Cohort Study,” Online, British Journal of Gynecology [BJOG] (Oxford, England) 126(1):24-31; Ueta, H., Kitazawa, Y., Sawanobori, Y., Ueno, T., Ueha, S., Matsushima, K., and Matsuno, K. 2018. Op cit. above in section 2a, Background of the Invention, General; Kruchen, A., Stahl. T., Gieseke, F. Binder, T. M. C., Oezcan, Z., and 10 others 2014. “Fetomaternal Microchimerism is Associated with Better Outcome in Haploidentical Hematopoietic Stem Cell Transplantation,” Blood 124(21):1242; Jackman, R. P. Z. Lee, T. H., and Busch, M. P. 2013. “Transfusion-associated Microchimerism: The Hybrid Within,” Transfusion Medicine Reviews 27(1):10-20; Reed, W. Lee. T. H. Norris, P. J., Utter, G. H., and Busch, M. P. 2007. “Transfusion-associated Microchimerism: A New Complication of Blood Transfusions in Severely Injured Patients,” Seminars in Hematology 44(1):24-31)

Guidance concerning circulatory support should pertain to both the status of the patient and the mechanical implementation of the process (see, for example, Murala, J. and Si, M. S. 2017. “Mechanical Circulatory Support for the Failing Functional Single Ventricle,” Translational Pediatrics 6(1):59-61; Bloch, E. M., Whyte, R. K. and Jefferies, A. L. 2014. “Red Blood Cell Transfusion in Newborn Infants,” Paediatric and Child Health 19(4):213-222; Pappas, A., Shankaran, S., Hansen, N., Bell, E. F., Stoll, B. J., and 12 others 2012. “Outcome of Extremely Preterm Infants (<1,000 g) with Congenital Heart Defects from the National Institute of Child Health and Human Development Neonatal Research Network,” Pediatric Cardiology 33(8):1415-1426; Kanji, H. D., Schulze, C. J., Oreopoulos, A., Lehr, E. J., Wang, W., and MacArthur, R. M. 2010. “Peripheral versus Central Cannulation for Extracorporeal Membrane Oxygenation: A Comparison of Limb Ischemia and Transfusion Requirements,” Thoracic and Cardiovascular Surgeon 58(8):459-462).

The preferability of metered to sudden compound vascular bypass, or sudden switched-organ transplantation, affording a gradually increased blending of recipient and donor blood therefore increases with each day after delivery. Donor-recipient immune harmonization through metered compound vascular bypass, or sudden switched-organ transplantation does not preclude the periprocedural application of manual means for the exchange of blood products and cells. Supplementation thus is appropriate when metered compound vascular bypass, or sudden switched-organ transplantation alone would require continuation over a duration considered excessive.

Another set of insufficiently correctable conditions notwithstanding improvements such as cavopulmonary anastomosis in Fontan, bidirectional Glenn, or Kawashima operations is presented by univentricular defects, where passive nonpulsatile Fontan flow to the lungs results in chronic hypoxia, sooner or later leading to globally impaired organ development if not failure, dysrhythmias, stroke, myocardial infarction, and reduced life expectancy (Kenny, L. A., DeRita, F., Nassar, M., Dark, J., Coats. L., and Hasan, A. 2018. “Transplantation in the Single Ventricle Population,” Annals of Cardiothoracic Surgery (Hong Kong, China) 7(1):152-159; Kverneland, L. S., Kramer, P., and Ovroutski, S. 2018. “Five Decades of the Fontan Operation: A Systematic Review of International Reports on Outcomes after Univentricular Palliation,” Congenital Heart Disease 13(2):181-193; Jeon, B. B., Park, C. S., and Yun, T. J. 2018. “Heart Transplantation in Patients with Superior Vena Cava to Pulmonary Artery Anastomosis: A Single-institution Experience,” Korean Journal of Thoracic and Cardiovascular Surgery 51(3):167-171; van der Ven, J. P. G., van den Bosch. E., Bogers, A. J. C. C., and Helbing, W. A. 2018. “State of the Art of the Fontan strategy for Treatment of Univentricular Heart Disease” F1000[Faculty of 1000] Research 7. pii: F1000 Faculty Rev-935: Hauck, A., Porta, N., Lestrud, S., and Berger, S. 2017. “The Pulmonary Circulation in the Single Ventricle Patient,” Children (Basel, Switzerland) 4(8). pii: E71; Cześniewicz, P. J. and Kusa. J. 2017. “Approaching the 50th Anniversary of the First Fontan Procedure. What is the Current State of Treatment Provided to Patients with Functional Single Ventricles?,” (in English) Polskie Towarzystwo Kardio-Torakochirurgów (Polish Journal of Cardiothoracic Surgery) 14(3):186-191; Caneo. L. F., Neirotti, R. A., Turquetto, A. L., and Jatene, M. B. 2016. “The Fontan Operation is Not the End of the Road” (in English and Portuguese). Arquivos Brasileiros de Cardiologia (Brazilian Archives of Cardiology, Sao Paulo, Brazil) 106(2):162-165; Mavroudis, C. 2012. “Fontan Conversion/Arrhythmia Surgery Long-term Database,” at http://childrensheartfoundation.org/node/314; Huebler, M., Schubert, S., Lehmkuhl, H. B., Weng, Y., Miera, O., and 3 others 2011. Op cit. above under 1. Background of the Invention. a. General; Michielon, G., Carotti, A., Pongiglione, G., Cogo, P., and Parisi, F. 2011. “Orthotopic Heart Transplantation in Patients with Univentricular Physiology,” Current Cardiology Reviews 7(2):85-91; Griffiths, E. R., Kaza, A. K., Wyler von Ballmoos, M. C., Loyola, H., Valente, A. M., Blume, E. D., and del Nido. P. 2009. “Evaluating Failing Fontans for Heart Transplantation: Predictors of Death,” Annals of Thoracic Surgery 88(2):558-564; Muller, J., Christov. F., Schreiber, C., Hess, J., and Hager, A. 2009. “Exercise Capacity, Quality of Life, and Daily Activity in the Long-term Follow-up of Patients with Univentricular Heart and Total Cavopulmonary Connection,” European Heart Journal 30(23):2915-2920; Michielon, G., Parisi. F., Di Carlo, D., Squitieri, C., Carotti, A., Buratta, M., and Di Donato, R. M. 2003. “Orthotopic Heart Transplantation for Failing Single Ventricle Physiology,” European Journal of Cardiothoracic Surgery 24(4):502-510; Michielon, G., Parisi. F., Squitieri, C., Carotti, A., Gagliardi, G., Pasquini. L., and Di Donato, R. M. 2003. “Orthotopic Heart Transplantation for Congenital Heart Disease: An Alternative for High-risk Fontan Candidates?,” Circulation 108 Supplement 1:11140-11149).

Clearly, means for facilitating and rendering less traumatic heart transplantation as soon following birth as possible is of great advantage. Where the need for lifelong immunosuppression-which can be directly pipeline targeted as not to render the body susceptible to disease—is less of a threat than is Fontan circulation, Fontan operations should be viewed as short-term life-saving measures pending replacement with a healthy heart.

Modified Fontan procedures—the Fontan Bjork and total cavopulmonary connection procedures-offer some pulsatile systolic pulmonary flow and succeed in reducing adverse sequelae, but insufficiently (Ono. M., Vogt. M., Cleuziou, J., Kasnar-Samprec. J., Burri. M., and 5 others 2016. “Improved Exercise Performance in Patients with Tricuspid Atresia after the Fontan-Björk Modification with Pulsatile Systolic Pulmonary Flow,” Annals of Thoracic Surgery 101(3):1012-1019). The ultimate fact is that no attempt at repair can even approximate the circulatory function of two normal ventricles (Erikssen, G., Aboulhosn, J., Lin, J., Liestol, K., Estensen, M. E., and 4 others 2018. “Survival in Patients with Univentricular Hearts: The Impact of Right Versus Left Ventricular Morphology,” Open Heart 5(2):e000902; Pessotti, C. F. X., Costa, P. R. S. M., Baranauskas, N. F. J., Correa, T. M., and Jatene, I. B. 2018. “Late Follow-up of Patients Submitted to Total Cavopulmonary Derivation: Clinical Aspects, Reinterventions, and Complications Interfering in Morbidity and Mortality,” Brazilian Journal of Cardiovascular Surgery 33(3):271-276).

Fontan procedures arose in a context of lacking the means for preventing the rejection of a replacement heart. With current immune capability, especially using direct pipeline targeting to spare other tissue exposure to the drugs, and directly switched transplantation, Fontan procedures yield passive, low-pressure, and nonpulsatile flow that results in chronic hypoxemia, and given more recent approaches, should be used only as a bridging strategy when the condition of the patient is grave and a replacement heart is unavailable.

Other causes for congenital heart malformation, sometimes severe as to recommend transplantation, arise with or secondarily to dysfunction during development elsewhere in the body, such as cardiorenal syndrome (Merz, W. M., Kübler, K., Fimmers. R., Willruth, A., Stoffel-Wagner, B., and Gembruch, U. 2013, Op cit. above at a. General. (1). Congenital Heart and Other Organ Defects: The Importance of Prompt Correction; Rosner, M. H., Rastogi. A., and Ronco, C. 2011 Op cit. above at a. General. (1). Congenital Heart and Other Organ Defects: the Importance of Prompt Correction).

Ordinarily, a prenate, neonate, or infant without a mature immune system and therefore without immunological complications such as maternal chimerization can have an organ replaced by sudden, without the need for metered, switch technique. When this is not the case, then prior to transplantation, the immune systems of mother, recipient, and donor may be preconditioned or partially harmonized by cross- or inter-chimerization beyond that spontaneously induced for mother-fetus tolerance (see, for example, Pan, H., Gazarian, A., Dubernard, J. M., Belot. A., Michallet, M. C., and Michallet, M. 2016. “Transplant Tolerance Induction in Newborn Infants: Mechanisms, Advantages, and Potential Strategies,” Online, Frontiers in Immunology (Lausanne, Switzerland) 7:116; Kitchens, W. H. and Adams, A. B. 2016. “Nonhuman Primate Models of Transplant Tolerance: Closer to the Holy Grail,” Current Opinion in Organ Transplantation 21(1):59-65; Avsar. M., Jansson, K., Sommer, W., Kruse. B., Thissen, S., and 16 others 2016. “Augmentation of Transient Donor Cell Chimerism and Alloantigen-Specific Regulation of Lung Transplants in Miniature Swine,” American Journal of Transplantation 16(5):1371-1382; Rezaee, F., Peppelenbosch, M., and Dashty, M. 2013, Op cit.; Pilat, N. and Wekerle, T. 2010. “Mechanistic and Therapeutic Role of Regulatory T Cells in Tolerance through Mixed Chimerism,” Current Opinion in Organ Transplantation 15(6):725-730; Wekerle, T. and Sykes, M. 1999. “Mixed Chimerism as an Approach for the Induction of Transplantation Tolerance,” Transplantation 68(4):459-467; Wood, K. J. 1996. “Induction of Tolerance to Cardiac Allografts,” Current Opinion in Cardiology 11(2):208-213). Favorable experimental results obtained with murids are not necessarily transferable to humans or are not transferable without additional measures.

The immune tolerance that suppresses mutual rejection between mother and child should not be depended upon to avoid this process. Among the vulnerabilities present are hemolytic disease (citations below) and increased susceptibility to infection. During the natal and early postnatal period, the immune tolerance of the fetus can prove devastating, making the need for cross-chimerization and sterility in the production and packaging of side-entry diversion jackets and asepsis during delivery and in the maternity ward imperative.

Thus, the need to cross-chimerize the fetus or neonate with both its mother and the donor must be acertained (see, for example, Guzman-Genuino, R. M. and Diener, K. R. 2017. “Regulatory B Cells in Pregnancy: Lessons from Autoimmunity, Graft Tolerance, and Cancer,” Online, Frontiers in Immunology (Lausanne, Switzerland) 8:172; Koehn, B. H. and Blazar, B. R. 2017. “Role of Myeloid-derived Suppressor Cells in Allogeneic Hematopoietic Cell Transplantation,” Online, Journal of Leukocyte Biology (Oxford, England) February 1. pii: ilb.5MR1116-464R; Wortel, C. M. and Heidt S. 2017. “Regulatory B cells: Phenotype, Function and Role in Transplantation,” Transplant Immunology 41:1-9; Schuetz, C., Lee, K. M., Scott. R., Kojima, L., Washburn, L., and 7 others 2017. “Regulatory B Cell-dependent Islet Transplant Tolerance is also Natural Killer Cell Dependent,” American Journal of Transplantation 17(6):1656-1662; Fragiadakis, G. K., Baca, Q. J., Gherardini, P. F., Ganio, E. A., Gaudilliere, D. K., and 16 others 2016. “Mapping the Fetomaternal Peripheral Immune System at Term Pregnancy,” Journal of Immunology (Baltimore) 197(11):4482-4492; Bézie, S., Picarda, E., Ossart. J., Martinet. B., Anegon, I., and Guillonneau, C. 2015. “Compensatory Regulatory Networks between CD8 T, B, and Myeloid Cells in Organ Transplantation Tolerance,” Joumal of Immunology (Baltimore) 195(12):5805-5815; La Rocca, C., Carbone, F., Longobardi, S., and Matarese, G. 2014. “The Immunology of Pregnancy: Regulatory T Cells Control Maternal Immune Tolerance toward the Fetus,” Immunology Letters 162(1 Part A): 41-48; Gervassi, A., Lejarcegui, N., Dross, S., Jacobson, A., Itaya, G., and 4 others 2014. “Myeloid Derived Suppressor Cells are Present at High Frequency in Neonates and Suppress In Vitro T Cell Responses,” Online, PLoS [Public Library of Science] One 9(9):e107816; Alijotas-Reig. J., Llurba, E., and Gris, J. M. 2014. “Potentiating Maternal Immune Tolerance in Pregnancy: A New Challenging Role for Regulatory T Cells,” Placenta 35(4):241-248; Zenclussen, A. C. 2013. “Adaptive Immune Responses during Pregnancy,” American Journal of Reproductive Immunology (New York, New York) 69(4):291-303; Lan, F., Zeng. D., Higuchi, M., Higgins, J. P., and Strober, S. 2003. “Host Conditioning with Total Lymphoid Irradiation and Antithymocyte Globulin Prevents Graft-versus-host Disease: The Role of CDI-reactive Natural Killer T Cells,” Biology of Blood and Marrow Transplantation 9(6):355-363).

Apart from the donor, any genetic disorders of the recipient detectable in utero that might compromise success are treated prenatally by then proven methods of stem cell transplantation and/or gene therapy (see, for example, Almeida-Porada, G., Atala, A., and Porada. C. D. 2016. “In Utero Stem Cell Transplantation and Gene Therapy: Rationale, History, and Recent Advances Toward Clinical Application,” Molecular Therapy. Methods and Clinical Development 5: 16020; McClain, L. E. and Flake, A. W. 2016, Op cit.; Ramachandra, D. L., Shaw, S. S., Shangaris, P., Loukogeorgakis, S., Guillot, P. V., Coppi, P. D., and David, A. L. 2014. “In Utero Therapy for Congenital Disorders Using Amniotic Fluid Stem Cells,” Online, Frontiers in Pharmacology 5:270; Loukogeorgakis, S. P. and Flake, A. W. 2014, Op cit.; Vrecenak, J. D. and Flake, A. W. 2013. “In Utero Hematopoietic Cell Transplantation-Recent Progress and the Potential for Clinical Application,” Cryotherapy 15(5):525-535; Pearson, E. G. and Flake. A. W. 2013. “Stem Cell and Genetic Therapies for the Fetus,” Seminars in Pediatric Surgery 22(1):56-61; Strober, S. 2008. “Protective Conditioning against GVHD and Graft Rejection after Combined Organ and Hematopoietic Cell Transplantation,” Blood Cells, Molecules, and Diseases 40(1):48-54).

There are, however, notable exceptions to mother-fetus immune accommodation which untreated are deadly but can be controlled, so that whether to proceed with transplantation in this circumstance is necessarily a clinical judgment (see, for example, Hendrickson, J. E. and Delaney, M. 2016. “Hemolytic Disease of the Fetus and Newborn: Modern Practice and Future Investigations,” Transfusion Medicine Reviews 30(4):159-164; de Haas, M., Thurik, F. F., van der Ploeg. C. P., Veldhuisen, B., Hirschberg. H., and 6 others 2016. “Sensitivity of Fetal RHD [Rhesus factor gene D] Screening for Safe Guidance of Targeted Anti-D Immunoglobulin Prophylaxis: Prospective Cohort Study of a Nationwide Programme in the Netherlands,” Online, British Medical Journal 355:15789; Lukacevic Krstic, J., Dajak, S., Bingulac-Popovic, J., Dogic, V., and Mratinovic-Mikulandra, J. 2016. “Anti-D Antibodies in Pregnant D Variant Antigen Carriers Initially Typed as RhD,” Transfusion Medicine and Hemotherapy 43(6):419-424; Hendrickson, J. E., Tormey, C. A., and Shaz, B. H. 2014. “Red Blood Cell Alloimmunization Mitigation Strategies,” Transfusion Medicine Reviews 28(3):137-144; Basu, S., Kaur. R., and Kaur, G. 2011. “Hemolytic Disease of the Fetus and Newborn: Current Trends and Perspectives,” Asian Journal of Transfusion Science 5(1):3-7; Urbaniak, S. J. and Greiss, M. A. 2000. “RhD [Rhesus blood group. D antigen] Haemolytic Disease of the Fetus and the Newborn,” Blood Reviews 14(1):44-61). The essential factor appurtenant of delivery is separation from the mother.

Because ductus side-entry jackets and valve-jackets allow the direct delivery of drugs into the substrate vessel or ureter, for example, and nonjacketing side-entry connectors can release drugs into any subjacent or substrate tissue, any pathophysiological change which the sensors can detect will be responded to immediately. Mediated by an automatic disorder response system and servovalve-jackets, the attempt to transplant the heart of a chimpanzee or a baboon into a child, for example, would be terminated as soon as the buildup in M-, G-, and A-class xenoreactive immunoglobulin natural antibodies-cooriginal with colonization of the neonate intestine by bacteria with cell walls surmounted by galactose moieties, signaling impending hyperacute rejection, was detected. Chimerization and immunosuppression notwithstanding, such rejection, compounded with ischemia-reperfusion injury, the short lifespan of other mammals, and the risk of zoonotic transmission, give xenogeneic transplantation, or xenotransplantation, little prospect for success for a long time.

However, if these problems can be overcome, the application of metered compound vascular bypass, or sudden switched-organ transplantation thereto, because it: 1. Eliminates ischemia-reperfusion injury. 2. Is able to gradualize the concurrent transfer of the organ and blending of blood between recipient and donor, and 3. Will be administered by a totally implanted prosthetic disorder response system able to detect and immediately react to any reversal, will facilitate if not enable such success (see, for example Kim, I. C., Youn, J. C., and Kobashigawa, J. A. 2018. “The Past. Present and Future of Heart Transplantation,” Korean Circulation Journal 48(7):565-590, Op cit., sections 2a and 2a(3); Meier, R. P. H., Muller, Y. D., Balaphas, A., Morel, P., Pascual, M., Seebach, J. D., and Buhler, L. H. 2018. “Xenotransplantation: Back to the Future?,” Transplant International 31(5):465-477; De Los Angeles, A., Pho, N., and Redmond, D. E. Jr. 2018. “Generating Human Organs via Interspecies Chimera Formation: Advances and Barriers,” Yale Journal of Biology and Medicine 91(3):333-342; Wu, J., Platero-Luengo, A., Sakurai, M., Sugawara, A., Gil, M. A., and 32 others 2017. “Interspecies Chimerism with Mammalian Pluripotent Stem Cells,” Cell 168(3):473-486.e15; Oldani, G., Peloso, A., Lacotte, S., Meier, R., and Toso, C. 2017. “Xenogeneic Chimera-Generated by Blastocyst Complementation—as a Potential Unlimited Source of Recipient-tailored Organs,” Xenotransplantation 24(4); Puga Yung, G. L., Rieben, R., Bühler. L., Schuurman, H. J., and Seebach, J. 2017. “Xenotransplantation: Where Do We stand in 2016?,” Swiss Medical Weekly 147: w14403; Hryhorowicz, M., Zeyland, J., Słomski, R., and Lipiński, D. 2017. “Genetically Modified Pigs as Organ Donors for Xenotransplantation,” Molecular Biotechnology 59(9-10):435-444; Ekser. B., Li, P., and Cooper, D. K. C. 2017. “Xenotransplantation: Past. Present, and Future,” Current Opinion in Organ Transplantation 22(6):513-521; Cooper, D. K. C. 2017. “Early Clinical Xenotransplantation Experiences—An Interview with Thomas E. Starzl, MD. PhD,” Xenotransplantation 24(2); Wu. J. and Izpisua Belmonte, J. C. 2016. “Interspecies Chimeric Complementation for the Generation of Functional Human Tissues and Organs in Large Animal Hosts,” Transgenic Research 25(3):375-384; Boksa. M., Zeyland, J., Słomski, R., and Lipinski, D. 2015. “Immune Modulation in Xenotransplantation,” Archivum Immunologiae et Therapiae Experimentalis (Warsaw, Poland) 63(3):181-192; Zeyland, J., Lipinski, D., and Słomski, R. 2015. “The Current State of Xenotransplantation,” Journal of Applied Genetics 56(2):211-218; Cooper, D. K. C., Ekser, B., and Tector, A. J. 2015. “A Brief History of Clinical Xenotransplantation,” International Journal of Surgery 23 (Part B): 205-210; Cooper, D. K. C. 2012. “A Brief History of Cross-species Organ Transplantation,” Proceedings of the Baylor University Medical Center 25(1):49-57).

Where the primary cause of disease is elsewhere in the body with a secondary effect exerted at the site of the jacket, the prescription-program is written to target the original root-site or site of the origin of disease. This immediacy of detection and medicinal response can render transplantable organs that would otherwise be considered too risky. For this reason, the use of an implanted automatic disorder response system can expand the zone of acceptability for donor organs that would otherwise be discarded as posing an inordinate likelihood of being rejected.

With the organs and tissues of fetuses sustained intact by somatic support initiated following induced delivery or Caesarean section immediately after death and periodic blood replacement to flush through pump-damaged blood cells and detritus in regional organ banks at or adjacent to tertiary or quaternary centers with transplantation specialists, not by cryopreservation or freezing which imparts irreversible damage. (see, for example, Bruinsma, B. G. and Uygun, K. 2017. “Subzero Organ Preservation: The Dawn of a New Ice Age?,” Current Opinion in Organ Transplantation 22(3):281-286; Manuchehrabadi, N., Gao, Z., Zhang, J., Ring, H. L., Shao, Q., and 8 others 2017. “Improved Tissue Cryopreservation Using Inductive Heating of Magnetic Nanoparticles,” Science Translational Medicine 9(379) pii: eaah4586; Solanki, P. K., Bischof, J. C., and Rabin, Y. 2017. “Thermo-mechanical Stress Analysis of Cryopreservation in Cryobags and the Potential Benefit of Nanowarming,” Cryobiology 76:129-139; Lewis, J. K., Bischof, J. C., Braslavsky, I., Brockbank, K. G., Fahy, G. M., and 7 others 2016. “The Grand Challenges of Organ Banking: Proceedings from the First Global Summit on Complex Tissue Cryopreservation,” Cryobiology 72(2):169-182; Eisenberg, D. P., Bischof. J. C., and Rabin, Y. 2016. “Thermomechanical Stress in Cryopreservation Via Vitrification with Nanoparticle Heating as a Stress-Moderating Effect” Journal of Biomechanical Engineering 138(1); Feig, J. S., Eisenberg, D. P., and Rabin, Y. 2016. “Polarized Light Scanning Cryomacroscopy, Part I: Experimental Apparatus and Observations of Vitrification, Crystallization, and Photoelasticity Effects,” Cryobiology 73(2):261-271; Feig, J. S., Solanki, P. K., Eisenberg, D. P., and Rabin, Y. 2016. “Polarized Light Scanning Cryomacroscopy, Part II: Thermal Modeling and Analysis of Experimental Observations,” Cryobiology 73(2):272-281; Wiwanitkit V. 2014. “Organ Retrieval and Banking in Brain Dead Trauma Patients,” Indian Journal of Anaesthesia 58(1):102; Sawhney, C., Kaur. M., Lalwani, S., Gupta, B., Balakrishnan, I., and Vij. A. 2013. “Organ Retrieval and Banking in Brain Dead Trauma Patients: Our Experience at level-1 Trauma Centre and Current Views,” Indian Journal of Anaesthesia 57(3):241-247; Baust, J. G., Gao. D., and Baust, J. M. 2009. “Cryopreservation: An Emerging Paradigm Change,” Organogenesis 5(3):90-96), and retrieving the hearts and lungs of those lost due to causes other than cardiac or pulmonary, the perpetual shortage of replacement hearts and lungs can be reduced if not eliminated.

Other than relatively simple repairs such as those of septal defects, for example, allowing a fetus with a severe heart malformity to come to delivery might allow irreversible multiple organ damage to already have occurred and forces complex attempts at repairing the heart, which can improve but not correct abnormal flow, and surgery to repair the other major organs. By contrast, in utero transplantation preempts secondary maldevelopment of the brain and other organs throughout the body, eliminating the need for high risk attempts at correction which can only palliate, not repair the heart too late to protect other vital organs. Moreover, because some three million stillbirths occur annually worldwide, the stringency of matching based upon anatomical considerations is reduced. These factors materially increase the number of replacement hearts available at the time when replacement is accomplished to the most beneficial and long-lasting effect.

Responsive to exigent need, compound vascular bypass double heart transplantation in a neonate is addressed below in section 2b(1)(b)(b5), entitled Heterotopic Double Heart Metered Compound Vascular Bypass, or Switched-organ Transplantation [and so on], and the Use of Unified Entry Arterial and Venous Exit Confluence Conduits to Avoid Disuse Atrophy when the Graft Organ is Positioned in the Lower Abdomen by Direct Aortocaval Connection, in the subsection at the end thereof entitled Heterotopic Double Heart Transplantation in a Prenate, Neonate, or Infant. In developed countries which permit abortion, the pool of available replacement hearts can be made substantial, eliminating such need.

2b(1)(b)(b4). Metered Compound Vascular Bypass, or Switched-Organ Transplantation in an Adult

The gradual and seamless transfer of a graft organ from the circulatory system of the donor into the circulatory system of the recipient makes possible not only a reduction in the abrupt confrontational nature of immediate organ replacement and therewith a factor of conditioning, but provides a period for the administration of collateral measures to include substances concurrent with cross-microchimerization during reciprocal cross-circulation such as those addressed above in section 2a, entitled Background of the Invention. General. Accessory or adjuvant substances can be added manually but supplementation thus would best be undertaken by the implanted automatic disorder response system with its rejection sensors to not only signal the intensification in the rejection response but automatically regulate the rate of substance addition in accordance with the system prescription-program.

The heart is unique in representing the central driver of a circuit wherein all venous blood must directly return to and all arterial blood be directly ejected from it. This applies no less to conventional heterotopic, or ‘piggyback,’ heart transplantation and to a double heart metered switch transplant, where both hearts must be situated at the origin and destination, at the center, of the circulatory system. Just as the growth-accommodating elastic and nonrupturable fabric patch aortoplasty and other major vasculature replacement fabric described below under Section 6, entitled Description of the Preferred Embodiments of the Invention, the subsection entitled Artificial Aorta and Its Automatic Placement, confluence conduits for placement in pediatric patients are made of the same fabric.

In neonates, the most common reason for the need of a new heart is a heart congenitally malformed to such a degree that even the most exceptional skill in its repair would not initiate normal pulsatile circulation. The immature immune system in neonates and infants with one or more malformed organs incapable of adequate repair-such as is commonly encountered in the heart with trisomy 21, for example—is advantageous in allowing organ replacement using the sudden switch method where compared to an older child or adult, the native organ can be replaced suddenly by the graft organ with a reduced risk of rejection. In adults, use of the sudden rather than the metered technique with its emphasis on immune tolerance induction is justified only in end stage heart failure with the risk of death imminent.

With a mature immune system, application of the sudden as opposed to the metered switch technique in adults is justified only in exigent circumstances such as when the native organ of the prospective recipient has been selectively and irremediably damaged by a disease as may be imposed on the heart by rheumatic fever or heart failure, or its function irremediably diminished as the result of accidental trauma where the remediation of any intercurrent disease would not result in improvement. That is, a replacement organ is justified when the disease or disorder was squarely contained within the native organ so that to replace that organ would likely bring not a cure but the approximation of a cure.

By contrast, uncured systemic disease and disease with a predilection for a particular organ, such as tuberculosis, which infectious, and Churg-Straus disease, which noninfectious, favors the lungs as to similarly affect a graft lung were it provided, contraindicate a lung transplant. Broadly, transplantation is indicated for end-stage failure due to disease whether benign or malignant when specific to the organ and contraindicated when secondary to systemic disease whether benign or malignant. Disseminated benign and metastasized conditions are counterindications to transplantation. Hence, mesothelioma and granulomatous disease but not advanced cystic fibrosis, pulmonary fibrosis, interstitial lung disease, pulmonary hypertension, or chronic occlusive pulmonary disease, for example, contraindicate lung transplantation, just as metastasized pancreatic cancer contraindicates pancreatic transplantation.

In addition to metastatic cancer, hematogenously disseminated and thus systemic, granulomatous disease whether infectious, noninfectious, benign, or malignant, deposits infiltrates in tissues making such conditions contraindicative for transplantation. Not classified as a granulomatous disease as such, systemic amyloidosis is also contraindicative for solid organ transplantation. Infectious granulomatous diseases include tuberculosis; Hansen's disease, or leprosy, progressive disseminated, chronic fibrocavitary pneumonia, or fibrosing mediastinum histoplasmosis; cryptococcosis, also called torulosis or European blastomycosis; coccidioidomycosis also called valley of San Joaquin, fever; blastomycosis, also called Gilchrist's disease or North American blastomycosis; and cat-scratch disease, or cat-fever.

With some conditions, such as tuberculosis, provided vaccination and booster doses precede immunosuppression, and the follow-up use of immunosuppressives rigorously adheres to the prescribed regimen, solid organ transplantation is not necessarily proscribed (see, for example Finberg, R. and Fingeroth, J. 2005. “Infections in Transplant Recipients,” in Kasper, D. L, Braunwald, E., Fauci, A. S., Hauser, S. L., Longo, D. L., and Jameson, J. L. Harrison's Principles of Internal Medicine, Chapter 117, pages 788 and 789, New York, New York: McGraw-Hill Medical Publishing Division).

Non-infectious granulomatous diseases include chronic lymphocytic leukemias such as mycosis fungoides and Sezary syndrome; sarcoidosis; Wegener's granulomatosis; Churg-Strauss granulomatosis, or syndrome, or allergic angiitis; berylliosis as chronic beryllium disease; Crohn's disease; and the development of rheumatoid nodules in the lungs (see, for example, The Merck Manual, 2006, Whitehouse Station, New Jersey: Merck Research Laboratories Division, Merck and Company, Inc.).

Unless death had been cardiogenic such as due to a sudden arrest, the heart continues to beat for a time past death so that the organs and tissues continue to be perfused, albeit without normal sinus rhythm or stability so that placing the deceased on somatic support with regular pulsation may continue the organs in a more favorable condition to be transplanted than if the heart had undergone final arrest. A standardized procedure would have required the procurement of the permissions necessary and the preservation of the entire body in a usable condition.

Intensive somatic support prior to brain death and preferably, well before the terminal event would then have been initiated with the placement of an implant system originally intended to optimize the life saving treatment of the patient and possibly avoid the need for an organ transplant. By placing the patient on life support before death, the organs and tissues are spared much trauma to include the forestalling of autophagy. Should the patient die, the system continues under the control of a prescription-program that had been written at the outset to sustain the optimal overall condition of the remains in the event of death.

With sudden compound vascular bypass, or sudden switched-organ transplantation, switching the replacement organ of the deceased donor into circulatory system of the recipient is abrupt rather than gradual, precluding the midprocedural administration of cellular materials from the donor when available. This is done manually with blood draws and laboratory analysis, for example, before and after the switch is made. End-stage disease of any major organ will have exerted a degenerative effect throughout the body. Given the interdependence of organs, organ systems, and tissues, for a condition of one organ to not have affected the others would be found only in the earliest stages and then infrequently, as in cases of accidental death.

As a result, adults, with a mature immune system, almost all of whom present with comorbidities, hence, sensitized antibodies, should be treated using metered switch technique. In an emergency, the primary deterrents to promptness in organ replacement are organ acquisition and the need to attend to any other injuries or disease of the prospective recipient when received in the emergency room. When the patient can be supported for two hours before initiating the transplant, any substance in the recipient that could detract from acceptance or impair the condition of the new heart which can should be removed through apheresis, the administration of a reversal agent, and/or drugs. Regardless of which organ must be replaced, heart failure and associated comorbidities can influence pharmacokinetics, for example.

The improved prospects for a successful transplant are much increased when an implanted response system automatically and directly pipeline targets anti-inflammatoires and other supportive medication to the new organ, here the heart, and the value of such a system is heightened with the addition of one or more comorbid conditions best treated in an immediate and coordinated manner which periodic visits to the clinic cannot approximate. Dependency upon patient compliance eliminated, support thus enhances the prospect for a good outcome and removes a central deterrent to transplantation as a treatment for heart failure, for example.

For end-stage heart failure with dilated rather than ischemic cardiomyopathy, the greater unpredictability of volume reduction surgery by Batista partial left ventriculectomy or aneurysmal repair through a Dor endoventricular circular patch plasty, should be significantly lessened with improved outcomes when overseen by an implanted automatic monitoring and response system capable of direct-to-root root, or site, of disease pipeline targeting of medication immediately upon detection. Such a more recent development can secondarily and retroactively allow the realization of inherent potential in therapeutic measures that had been judged insufficiently effective and therefore overlooked if not abandoned. Such does not discount the added benefit of transcatheteric or transluminal annuloplasty to eliminate mitral regurgitation and/or angioplasty if ischemic cardiomyopathy is overlapping.

And if unsuccessful, then having used the prosthetic disorder response system as a medical management bridging strategy, the same system is used to accomplish switch transplantation of the diseased heart, here improving the odds for a more favorable outcome as well (see, for example, Sui, Y., Teng. S., Qian, J., Zhao, Z., Zhang, Q., and Wu, Y. 2019. “Treatment Outcomes and Therapeutic Evaluations of Patients with Left Ventricular Aneurysm,” Journal of International Medical Research 47(1):244-251; Chancellor, W. Z., Schubert, S. A., and Ailawadi, G. 2018. “Transcatheter Interventions for Functional Mitral Regurgitation,” Annals of Cardiothoracic Surgery (Hong Kong, China) 7(6):764-770; Kaya, U., çolak, A., Becit. N., Ceviz, M., and Kocak, H. 2018. “Application of Circular Patch Plasty (Dor Procedure) or Linear Repair Techniques in the Treatment of Left Ventricular Aneurysms,” Brazilian Journal of Cardiovascular Surgery (São Paulo, Brazil) 33(2):135-142; Wyler von Ballmoos, M. C., Kalra, A., and Reardon, M. J. 2018. “Complexities of Transcatheter Mitral Valve Replacement (TMVR) and Why It is Not Transcatheter Aortic Valve Replacement (TAVR),” Annals of Cardiothoracic Surgery (Hong Kong, China) 7(6):724-730; Domingues, J. S., Vale Mde, P., and Barbosa, M. P. 2015. “Partial Left Ventriculectomy: Have Well-succeeded Cases and Innovations in the Procedure Been Observed in the Last 12 Years?,” Brazilian Journal of Cardiovascular Surgery 30(5):579-585; Nishimura, M. 2012. “Surgical Treatment for Severe Heart Failure: Recent Progress in Treatment Strategy and Its Present Status in Japan,” Yonago Acta Medica 55(3):49-56; Letsou, G. V., Forrester, M., and Frazier, O. H. 2011. “Long-term Results of Complex Left Ventricular Reconstruction Surgery: Case Report,” Texas Heart Institute Journal 38(4):418-420; Suma, H. 2009. “Partial Left Ventriculectomy,” Circulation Journal 73 Supplement A: A19-A22; Matsui, Y. 2009. “Overlapping Left Ventricular Restoration,” Circulation Journal 73 Supplement A: A13-A18; Isomura, T. 2009. “Surgical Treatment for Heart Failure: Left Ventricular Restoration for Cardiomyopathy,” Circulation Journal 73 Supplement A: A6-A12; Di Donato, M., Castelvecchio, S., and Menicanti, L. 2009. “Surgical Treatment of Ischemic Heart Failure: The Dor Procedure,” Circulation Journal 73 Supplement A: A1-A5; Sartipy, U., Kjellman. U., Samuelsson, S., Hagerman, I., Wikström, G., and 3 others 2006. “Left Ventricular Reconstruction as an Alternative to Heart Transplantation: A Case Report,” Heart Surgery Forum 9(3):E638-E640; Hsu, R. B., Sartipy, U., Albage, A., and Lindblom, D. 2005. “The Dor Procedure for Left Ventricular Reconstruction. Ten-year Clinical Experience,” European Journal of Cardiothoracic Surgery 27(6):1005-1010; Chien, C. Y., Wang, S. S., and Chu, S. H. 2002. “Nontransplant Cardiac Surgery as a Bridge to Heart Transplantation in Pediatric Dilated Cardiomyopathy,” Texas Heart Institute Journal 29(3):213-215; Franco-Cereceda, A., Mccarthy, P. M., Blackstone, E. H., Hoercher, K. J., White, J. A., Young, J. B., and Starling. R. C. 2001. “Partial Left Ventriculectomy for Dilated Cardiomyopathy: Is this an Alternative to Transplantation?,” Journal of Thoracic and Cardiovascular Surgery 121(5):879-893; Dreyfus, G. and Mihealainu, S. 2001. “The Batista Procedure,” Heart 85(1):1-2; Chang, B. C., Lim, C. Y., Park, P. W., Park, K. Y., Lee, Y. T., and Kim, Y. J. 2001. “Volume Reduction Surgery for End-stage Heart Failure: Experience in Korea,” Journal of Cardiac Surgery 16(2):159-164; Suma, H. 2001. “Left Ventriculoplasty for Nonischemic Dilated Cardiomyopathy,” Seminars in Thoracic and Cardiovascular Surgery 13(4):514-521; Etoch, S. W., Koenig, S. C., Laureano, M. A., Cerrito, P., Gray, L. A., and Dowling, R. D. 1999. “Results after Partial Left Ventriculectomy versus Heart Transplantation for Idiopathic Cardiomyopathy,” Journal of Thoracic and Cardiovascular Surgery 117(5):952-959; Bolling, S. F., Pagani, F. D., Deeb, G. M., and Bach, D. S. 1998. “Intermediate-term Outcome of Mitral Reconstruction in Cardiomyopathy,” Journal of Thoracic and Cardiovascular Surgery 115(2):381-388).

The system can support other procedures such as a Konno or a Ross-Konno operation and the more so should these eventuate to have served as bridges to transplantation (see, for example, Yoon, D. W., Yang, J. H., Jun, T. G., and Park, P. W. 2017. “The Ross Procedure in Pediatric Patients: A 20-Year Experience of Ross Procedure in a Single Institution,” Korean Journal of Thoracic and Cardiovascular Surgery 50(4):235-241; Schneider, A. W., Bokenkamp, R., Bruggemans. E. F., and Hazekamp, M. G. 2016. “Twenty-year Experience with the Ross-Konno Procedure,” European Journal of Cardiothoracic Surgery 49(6):1564-1570; Mavroudis, C., Mavroudis, C. D., and Jacobs, J. P. 2014. “The Ross, Konno, and Ross-Konno Operations for Congenital Left Ventricular Outflow Tract Abnormalities,” Cardiology in the Young (Cambridge, England) 24(6):1121-1133; Aeba. R., Okamoto, K., and Yozu, R. 2010. “Ross-Konno Operation for Patients with Shone Complex,” Texas Heart Institute Journal 37(2):240-241; Reddy. V. M., Rajasinghe, H. A., Teitel, D. F., Haas, G. S., and Hanley, F. L. 1996. “Aortoventriculoplasty with the Pulmonary Autograft: The “Ross-Konno” Procedure,” Journal of Thoracic and Cardiovascular Surgery 111(1):158-167).

Especially Since VENTRICULAR TACHYARRHYTHMIA is a Sequela that the System WITH DEFIBRILLATOR

The placement of such a system prior to heart transplantation administers the transplantation and unless the other organs are transplanted as well, remains to treat the residual comorbid conditions with the object of seeking the optimal overall homeostatic result as can best be managed. In such a complex challenge to an internist, the continuous diagnostics fed by the implanted sensors to the control microprocessor and automatic release of drugs to each site (nidus, root) of disease in accordance with a prescription-program meticulously prepared in advance eliminates the need for exigent human response.

When a replacement organ is about to be introduced into a body with residually impaired organs, the interdependence of these argues for intensive support not limited to the immediate trauma and if possible, the preferability for metered compound vascular bypass, or sudden switched-organ transplantation in an adult. While necessitating the placement of additional sensors to monitor multiple locations for the detection of out of range values, the patient with plural interactive conditions of disease is better served by an automatic system that preserves optimal overall homeostasis through the immediate release of stabilizing agents.

Due to its prevalence, preexisting congestive heart disease can serve as a case in point (see, for example, Mangoni, A. A. and Jarmuzewska, E. A. 2019. “The Influence of Heart Failure on the Pharmacokinetics of Cardiovascular and Non-cardiovascular Drugs: A Critical Appraisal of the Evidence,” British Journal of Clinical Pharmacology 85(1):20-36; Serfas. J. D., Patel, P. A., and Krasuski, R. A. 2018, Op cit., above under section 2a(3), entitled Remotely Procured Graft Organ Preservation; Hörer, J. 2018. “Current Spectrum, Challenges and New Developments in the Surgical Care of Adults with Congenital Heart Disease,” Cardiovascular Diagnosis and Therapy 8(6):754-764; Krzysztofik, J. and Ponikowski, P. 2017. “Current and Emerging Pharmacologic Options for the Management of Patients with Chronic and Acute Decompensated Heart Failure,” Expert Review of Clinical Pharmacology 10(5): 517-534; van der Wal, H. H., Grote Beverborg, N., van Veldhuisen, D. J., Voors. A. A., and van der Meer, P. 2016. “Pharmacotherapy for Comorbidities in Chronic Heart Failure: A Focus on Hematinic Deficiencies, Diabetes Mellitus and Hyperkalemia,” Expert Opinion on Pharmacotherapy 17(11):1527-1538; Patel, P. A., Liang, L., Khazanie, P., Hammill, B. G., Fonarow, G. C. and 4 others 2016. “Antihyperglycemic Medication Use among Medicare Beneficiaries with Heart Failure, Diabetes Mellitus, and Chronic Kidney Disease,” Circulation. Heart Failure 9(7). pii: e002638; Lainscak, M., Vitale, C., Seferovic, P., Spoletini, I., Cvan Trobec, K., and Rosano, G. M. 2016. “Pharmacokinetics and Pharmacodynamics of Cardiovascular Drugs in Chronic Heart Failure,” International Journal of Cardiology 224:191-198; Lother, A. and Hein, L. 2016. “Pharmacology of Heart Failure: From Basic Science to Novel Therapies,” Pharmacology and Therapeutics 166:136-149).

End-stage heart failure patients will already have been taking some combination of a beta blocker, calcium channel blocker, a nitrate, typically, nitroglycerine, aldosterone antagonist, other diuretic, angiotensin converting enzyme inhibitor or angiotensin II receptor blocker, other vasodilator, digitalis or digoxin, metformin or insulin, and often, an anticoagulant. Drugs that could adversely affect the acceptance of a replacement heart should be reviewed for elimination (see, for example, van Middelaar, T. and Moll van Charante, E. P. 2018. “Deprescribing Preventive Medication in Older Patients,” British Journal of General Practice 68(675):456-457; Rankin, A., Cadogan, C. A., Patterson, S. M., Kerse, N., Cardwell, C. R., Bradley, M. C., Ryan, C., and Hughes, C. 2018. “Interventions to Improve the Appropriate Use of Polypharmacy for Older People,” Cochrane Database of Systematic Reviews 9: CD008165; Jordan, S., Logan, P. A., Panes, G., Vaismoradi, M., and Hughes, D. 2018. “Adverse Drug Reactions, Power, Harm Reduction, Regulation and the ADRe [adverse drug reaction] Profiles,” Pharmacy (Basel, Switzerland) 6(3). pii: E102: Triantafylidis, L. K., Hawley, C. E., Perry, L. P., and Paik, J. M. 2018. “The Role of Deprescribing in Older Adults with Chronic Kidney Disease,” Drugs and Aging 35(11):973-984; Levy, H. B. 2017. “Polypharmacy Reduction Strategies: Tips on Incorporating American Geriatrics Society Beers and Screening Tool of Older People's Prescriptions Criteria,” Clinics in Geriatric Medicine 33(2):177-187; Masnoon, N., Shakib, S., Kalisch-Ellett, L., and Caughey, G. E. 2017. “What is Polypharmacy? A Systematic Review of Definitions,” Online, BioMed Central Geriatrics 17(1):230; Shantsila, E. and Lip. G. Y. 2016. “Antiplatelet versus Anticoagulation Treatment for Patients with Heart Failure in Sinus Rhythm,” Cochrane Database of Systematic Reviews 9: CD003333; Alldred, D. P., Kennedy, M. C., Hughes, C., Chen, T. F., and Miller, P. 2016. “Interventions to Optimise Prescribing for Older People in Care Homes,” Cochrane Database of Systematic Reviews 2: CD009095; Cooper, J. A., Cadogan, C. A., Patterson, S. M., Kerse, N., Bradley, M. C., Ryan, C.1, and Hughes, C. M. 2015. “Interventions to Improve the Appropriate Use of Polypharmacy in Older People: A Cochrane Systematic Review,” Online, British Medical Journal Open 5(12):e009235; Zeitler, E. P. and Eapen, Z. J. 2015. “Anticoagulation in Heart Failure: A Review,” Journal of Atrial Fibrillation 8(1):1250; Gurbel, P. A. and Tantry, U. S. 2014. “Antiplatelet and Anticoagulant Agents in Heart Failure: Current Status and Future Perspectives,” Journal of the American College of Cardiology. Heart Failure 2(1):1-14).

Typically, renal, hepatic, pulmonary, and other organ injury will also be present as secondary consequences of the protracted cardiac impairment (see, for example, Forman, D. E., Maurer, M. S., Boyd. C., Brindis, R., Salive, M. E., and 6 others 2018. “Multimorbidity in Older Adults with Cardiovascular Disease,” Journal of the American College of Cardiology 71(19):2149-2161; Maack, C., Lehrke, M., Backs, J., Heinzel, F. R., Hulot, J. S., and 30 others 2018. “Heart Failure and Diabetes: Metabolic Alterations and Therapeutic Interventions: A State-of-the-art Review from the Translational Research Committee of the Heart Failure Association-European Society of Cardiology,” European Heart Journal 39(48):4243-4254; Kaur. P., Saxena, N., You, A. X., Wong, R. C. C., Lim, C. P., Loh, S. Y., and George, P. P. 2018. “Effect of Multimorbidity on Survival of Patients Diagnosed with Heart Failure: A Retrospective Cohort Study in Singapore,” Online, British Medical Journal Open 8(5):e021291; Tromp. J., Tay, W. T., Ouwerkerk, W., Teng. T. K., Yap. J., and [per erratum] 7 others 2018. “Multimorbidity in Patients with Heart Failure from 11 Asian Regions: A Prospective Cohort Study Using the ASIAN-HF [Asian Sudden Cardiac Death in Heart Failure] Registry,” Online, PLoS [Public Library of Science] Medicine 15(3):e1002541; Ferrucci. L. and Fabbri, E. 2018. “Inflammageing: Chronic Inflammation in Ageing, Cardiovascular Disease, and Frailty,” Nature Reviews Cardiology 15(9):505-522; Del Pinto, R. and Ferri, C. 2018. “Inflammation-accelerated Senescence and the Cardiovascular System: Mechanisms and Perspectives,” Online, MDPI [Multidisciplinary Digital Publishing Institute] International Journal of Molecular Sciences (Basel, Switzerland) 19(12). pii: E3701; Donato, A. J., Machin, D. R., and Lesniewski, L. A. 2018. “Mechanisms of Dysfunction in the Aging Vasculature and Role in Age-related Disease,” Circulation Research 123(7):825-848; lyngkaran, P., Liew. D., Neil, C., Driscoll, A., Marwick, T. H., and Hare, D. L. 2018. “Moving from Heart Failure Guidelines to Clinical Practice: Gaps Contributing to Readmissions in Patients with Multiple Comorbidities and Older Age,” Clinical Medicine Insights. Cardiology 12:1179546818809358; van der Wal, H. H., van Deursen, V. M., van der Meer. P., and Voors. A. A. 2017. “Comorbidities in Heart Failure,” Handbook of Experimental Pharmacology 243:35-66; Lesniewski, L. A., Seals, D. R., Walker, A. E., Henson, G. D., Blimline, M. W., and 6 others 2017. “Dietary Rapamycin Supplementation Reverses Age-related Vascular Dysfunction and Oxidative Stress, while Modulating Nutrient-sensing. Cell Cycle, and Senescence Pathways,” Aging Cell 16(1):17-26; Stewart, S., Riegel. B., Boyd, C., Ahamed. Y., Thompson, D. R., and 11 others 2016. “Establishing a Pragmatic Framework to Optimise Health Outcomes in Heart Failure and Multimorbidity (ARISE-HF) [five key steps proposed to improve the treatment of heart failure: Acknowledge multimorbidity, routine profiling thereof, identify individualised priorities and person-centered goals based on the extent and nature of multimorbidity, support individualized, home-based, multidisciplinary case management to supplement standard HF management, and evaluate health outcomes well beyond acute hospitalization and encompass all-cause events and a person-centered perspective in affected individuals]: A Multidisciplinary Position Statement,” International Journal of Cardiology 212:1-10; Willadsen, T. G., Bebe, A., Koster-Rasmussen, R., Jarbol, D. E., Guassora, A. D., and 3 others 2016. “The Role of Diseases, Risk Factors and Symptoms in the Definition of Multimorbidity—A Systematic Review,” Scandinavian Journal of Primary Health Care 34(2):112-121; RuDusky, B. M. 2015. “Heart Failure and Comorbidities,” Journal of the American College of Cardiology. Heart Failure 3(12):1003; Chong, V. H., Singh, J., Parry, H., Saunders, J., Chowdhury, F., Mancini, D. M.4, and Lang. C. C. 2015. “Management of Noncardiac Comorbidities in Chronic Heart Failure,” Cardiovascular Therapeutics 33(5):300-315; Luscher, T. F. 2015. “Heart Failure and Comorbidities: Renal Failure, Diabetes, Atrial Fibrillation, and Inflammation,” European Heart Journal 36(23):1415-1417; Mentz, R. J., Kelly, J. P., von Lueder, T. G., Voors. A. A., Lam, C. S., and 9 others 2014. “Noncardiac Comorbidities in Heart Failure with Reduced versus Preserved Ventricular Ejection Fraction,” Journal of the American College of Cardiology 64(21):2281-2293; Mentz, R. J., Kelly, J. P., von Lueder, T. G., Voors. A. A., Lam, C. S. and 9 others 2014. “Noncardiac Comorbidities in Heart Failure with Reduced versus Preserved Ventricular Ejection Fraction,” Journal of the American College of Cardiology 64(21):2281-2293; Widmer, F. 2011. “Comorbidity in Heart Failure,” (in German with abstract at Pubmed) Therapeutische Umschau (Therapeutic Review) 68(2):103-106; Lang, C. C. and Mancini, D. M. 2007. “Non-cardiac Comorbidities in Chronic Heart Failure,” Heart (London, England) 93(6):665-671; Dahlström, U. 2005. “Frequent Non-cardiac Comorbidities in Patients with Chronic Heart Failure,” European Journal of Heart Failure 7(3):309-316).

Immunological tolerogenic induction conditioning of both donor and recipient through exchanged engraftment of the cellular materials of either and tolerance-inducing drug onset of action always a consideration—before and/or after sudden switch, and before, during, and after metered compound vascular bypass, or sudden switched-organ transplantation will usually include the following no one of which is exclusive of the others and/or the adjunctive use of drugs. Especially for sudden compound vascular bypass, or sudden switched-organ, transplantation, unless the donor and recipient are not too genetically disparate, the time of onset for immune tolerance induction to include medication to take effect becomes a central consideration. Such measures include:

a. Preoperative low dose radiation (see, for example, Jansson, K., Dreckmann, K., Sommer, W., Avsar, M., Salman, J., and 10 others 2017. “Splenocyte Infusion and Whole-body Irradiation for Induction of Peripheral Tolerance in Porcine Lung Transplantation: Modifications of the Preconditioning Regime for Improved Clinical Feasibility,” Transplantation Direct 3(7):e170; Nagasaki, K., Obara. H., Xiong. A., Kambham, N., Strober. S., Esquivel, C. O., and Millan, M. T. 2007. “Liver Allografts are Tolerogenic in Rats Conditioned with Posttransplant Total Lymphoid Irradiation,” Transplantation 84(5):619-628; Warnecke, G., Avsar, M., Morancho, M., Peters, C., Thissen, S., and 8 others 2006. “Preoperative Low-dose Irradiation Promotes Long-term Allograft Acceptance and Induces Regulatory T Cells in a Porcine Model of Pulmonary Transplantation,” Transplantation 82(1):93-101; Jin, T., Toki, J., Inaba, M., Sugiura, K., Fan, T., Yu. C., Lian, Z., and 6 others 2001. “A Novel Strategy for Organ Allografts Using Sublethal (7 Gy) Irradiation followed by Injection of Donor Bone Marrow Cells via Portal Vein,” Transplantation 71(12):1725-1731). Results obtained using porcine and murid models is not necessarily transferable to humans or is not transferable without modification.

b. Donor-recipient exchanged gene transfer, cell therapy, blood and/or hematopoietic stem cell transfusions, and engraftment (see, for example, Cai, S. and Chandraker, A. 2019. “Cell Therapy in Solid Organ Transplantation,” Current Gene Therapy, Online, 19(2):71-80; Garakani. R. and Saidi. R. F. 2017. “Recent Progress in Cell Therapy in Solid Organ Transplantation,” Online, International Journal of Organ Transplantation Medicine 8(3):125-131; Yolcu, E. S., Shirwan, H., and Askenasy, N. 2017. “Mechanisms of Tolerance Induction by Hematopoietic Chimerism: The Immune Perspective,” Online, Stem Cells Translational Medicine 6(3):700-712; Purroy, C., Fairchild, R. L., Tanaka, T., Baldwin, W. M. 3rd, Manrique, J., and 9 others 2017. “Erythropoietin Receptor-mediated Molecular Crosstalk Promotes T Cell Immunoregulation and Transplant Survival,” Journal of the American Society of Nephrology 28(8):2377-2392; Mousavi, S. A., Javadimoghadam, M., Ghavamzadeh, A., Alimoghaddam, K., Sayarifard, A., Ghaffari S H I, Chahardouli. B., and Basi, A. 2017. “The Relationship between STR-PCR [short tandem repeat-polymerase chain reaction] Chimerism Analysis and Chronic GvHD Following Hematopoietic Stem Cell Transplantation,” International Journal of Hematology-Oncology Stem Cell Research 11(1):24-29; Davey, M. G., Riley, J. S., Andrews, A., Tyminski, A., Limberis, M., and 6 others 2017. “Induction of Immune Tolerance to Foreign Protein via Adeno-associated Viral Vector Gene Transfer in Mid-gestation Fetal Sheep,” Online, PLoS [Public Library of Science] One 12(1):e0171132; McClain, L. E. and Flake, A. W. 2016. In Utero Stem Cell Transplantation and Gene Therapy: Recent Progress and the Potential for Clinical Application, “Best Practice and Research. Clinical Obstetrics and Gynaecology 31:88-98; Urschel, S. and West. L. J. 2016. Op cit. under (b). Metered compound vascular bypass, or switched-organ, transplantation above; Reis, M., Ogonek, J., Qesari, M., Borges, N. M., Nicholson, L., and 5 others 2016. “Recent Developments in Cellular Immunotherapy for HSCT [hematopoietic stem cell therapy]-associated Complications,” Online, Frontiers in Immunology (Lausanne, Switzerland) 2016 7:500; Almeida-Porada, G., Atala, A., and Porada. C. D. 2016. “In Utero Stem Cell Transplantation and Gene Therapy: Rationale, History, and Recent Advances toward Clinical Application,” Molecular Therapy. Methods and Clinical Development 5:16020; Jaime-Perez, J. C., Villarreal-Villarreal, C. D., Mendez-Ramirez, N., Vazquez-Garza, E., Salazar-Riojas, R., and Gomez-Almaguer, D. 2016. “Assessment of Immune Reconstitution Status in Recipients of a Successful Hematopoietic Stem Cell Transplant from Peripheral Blood after Reduced Intensity Conditioning,” Blood Cells, Molecides, and Diseases 58:52-56: Tonsho, M., Lee, S., Aoyama, A., Boskovic, S., Nadazdin, O., and 9 others 2015. “Tolerance of Lung Allografts Achieved in Nonhuman Primates via Mixed Hematopoietic Chimerism,” American Journal of Transplantation 15(8):2231-2239; Simancas-Racines, D., Osorio, D., Marti-Carvajal, A. J., and Arevalo-Rodriguez, I. 2015. “Leukoreduction for the Prevention of Adverse Reactions from Allogeneic Blood Transfusion,” Cochrane Database of Systematic Reviews(12):CD009745; Schweiger, M., Stiasny, B., Dave, H., Cavigelli-Brunner, A., Balmer, C., and 4 others 2015, Op cit.; Sachs, D. H., Kawai. T., and Sykes, M. 2014. “Induction of Tolerance through Mixed Chimerism,” Cold Spring Harbor Perspectives in Medicine 4(1):a015529; Hirani. R., Balogh, Z. J., Loukogeorgakis, S. P. and Flake, A. W. 2014. “In Utero Stem Cell and Gene Therapy: Current Status and Future Perspectives,” European Journal of Pediatric Surgery 24(3):237-245; Lott, N. J., Hsu. J. M., and Irving, D. O. 2014. “Leukodepleted Blood Components Do Not Remove the Potential for Long-term Transfusion-associated Microchimerism in Australian Major Trauma Patients,” Chimerism 5(3-4):86-93; Henderson, H. T., Canter, C. E., Mahle, W. T., Dipchand. A. I., LaPorte, K., and 5 others 2012. “ABO-incompatible Heart Transplantation: Analysis of the Pediatric Heart Transplant Study (PHTS) Database,” Journal of Heart and Lung Transplantation (New York, New York) 31(2):173-279; Pasquet, L., Joffre, O., Santolaria, T., and van Meerwijk, J. P. 2011. “Hematopoietic Chimerism and Transplantation Tolerance: A Role for Regulatory T Cells,” Online, Frontiers in Immunology (Lausanne, Switzerland) 2:80; Bloch, E. M., Jackman, R. P., Lee, T. H., and Busch, M. P. 2013. “Transfusion-associated Microchimerism: The Hybrid Within,” Transfusion Medicine Reviews 27(1):10-20; Saczkowski, R., Dacey, C., and Bernier, P. L. 2010, Op cit. above under (b4). Metered compound vascular bypass, or sudden switched-organ transplantation in an Adult; Devi, A. S. 2009. “Transfusion Practice in Orthotopic Liver Transplantation,” Indian Journal of Critical Care Medicine 13(3):120-128; West. L. J. 2005. “Targeting Antibody-mediated Rejection in the Setting of ABO-incompatible Infant Heart Transplantation: Graft Accommodation vs. B Cell Tolerance,” Current Drug Targets. Cardiovascular and Haematological Disorders 5(3):223-232). Citations for gravida to fetus microchimerism and the effect upon this of open fetal surgery are provided below.

c. Donor-derived regulatory dendritic cells restrained by nuclear receptor co-repressor 1 or thyroid-hormone- and retinoic-acid-receptor-associated co-repressor 1 protein. “Owing to their ability to regulate both innate and adaptive immune responses, DC are considered to play crucial roles in directing the alloimmune response towards transplant tolerance or rejection,” (Ezzelarab, M. and Thomson, A. W. 2011. “Tolerogenic Dendritic Cells and Their Role in Transplantation,” Seminars in Immunology 23(4):252-263)(see also, for example, Pilat, N., Steiner, R., and Sprent, J. 2023. “Treg Therapy for the Induction of Immune Tolerance in Transplantation—Not Lost in Translation?,” Online, MDPI [Multidisciplinary Digital Publishing Institute] International Journal of Molecular Sciences (Basel, Switzerland) 24(2):1752; Sen, K., Pati, R., Jha. A., Mishra, G. P., Prusty, S., and 7 others 2023. “NCoR1 [nuclear receptor co-repressor 1] Controls Immune Tolerance in Conventional Dendritic Cells by Fine-tuning Glycolysis and Fatty Acid Oxidation,” Online, Redox Biology 59:102575; Bourque, J. and Hawiger, D. 2022. “Variegate Outcomes of T Cell Activation by Dendritic Cells in the Steady State,” Online, Journal of Immunology (Baltimore, Maryland) 208(3):539-547; Ghobadinezhad, F., Ebrahimi, N., Mozaffari, F., Moradi, N., Beinanvand, S., and 7 others 2022. “The Emerging Role of Regulatory Cell-based Therapy in Autoimmune Disease,” Online, Frontiers in Immunology (Lausanne, Switzerland) 13:1075813; Huang X. Y., Jin, Z. K., Dou, M., Zheng, B. X., Zhao, X. R., and 5 others 2022. “Sinomenine Promotes Differentiation of Induced Pluripotent Stem Cells into Immature Dendritic Cells with High Induction of Immune Tolerance,” Online, World Journal of Stem Cells (Pleasanton, California) 14(8):599-615; Kouyoumdjian, A., Tchervenkov J., and Paraskeas, S., 2022. “TFNR2 [tumor necrosis factor receptor 2] in Ischemia-Reperfusion Injury, Rejection, and Tolerance in Transplantation,” Online, Frontiers in Immunology (Lausanne, Switzerland) 13:903913; Moatti, A. Debesset, A., Pilon, C., Beldi-Ferchiou, A. Leclerc, M., and 11 others 2022. “TFNR2 Blockade of Regulatory T Cells Unleashes an Antitumor Immune Response after Hematopoietic Stem-cell Transplantation,” Online, Journal for Immunotherapy of Cancer 10(4):e003508; Fortunato, M., Morali, K., Passeri. L., and Gregori, S. 2021. “Regulatory Cell Therapy in Organ Transplantation: Achievements and Open Questions,” Online, Frontiers in Immunology (Lausanne, Switzerland) 12:641596; Macedo, C., Tran, L. M., Zahorchak, A. F., Dai, H., Gu, K., and 9 others 2021. “Donor-derived Regulatory Dendritic Cell Infusion Results in Host Cell Cross-dressing and T Cell Subset Changes in Prospective Living Donor Liver Transplant Recipients,” Online, American Journal of Transplantation 21(7):2372-2386; Nakano, R., Tran, L. M., Geller, D. A., Macedo, C., Metes, D. M., and Thomson, A. W. 2021. “Dendritic Cell-mediated Regulation of Liver Ischemia-Reperfusion Injury and Liver Transplant Rejection” Online, Frontiers in Immunology (Lausanne, Switzerland) 12:705465; Que, W. and Li, X. K. 2021. “Regulatory T Cells for the Induction of Transplantation Tolerance,” Advances in Experimental Medicine and Biology 1278:289-302; Robertson, H., Li, J., Kim, H. J., Rhodes, J. W., Harman, A. N., Patrick, E., and Rogers, N. M., 2021. “Transcriptomic Analysis Identifies a Tolerogenic Dendritic Cell Signature,” Online, Frontiers in Immunology (Lausanne, Switzerland) 12:733231; Comi. M., Amodio, G., Passeri, L., Fortunato, M., Santoni de Sio, F. R., and 5 others 2020. “Generation of Powerful Human Tolerogenic Dendritic Cells by Lentiviral-mediated IL-10 Gene Transfer,” Online, Frontiers in Immunology (Lausanne, Switzerland) 12:672701; Iberg, C. A. and Hawiger, D. 2020. “Natural and Induced Tolerogenic Dendritic Cells,” Online, Journal of Immunology (Baltimore, Maryland) 204(4):733-744; Ochando, J., Ordikhani. F., Jordan, S., Boros, P., and Thomson, A. W. 2020. “Tolerogenic Dendritic Cells in Organ Transplantation,” Transplant International 33(2):113-127; Que, W., Guo, W. Z., and Li, X. K. 2020. “Manipulation of Regulatory Dendritic Cells for Induction Transplantation Tolerance,” Online, Frontiers in Immunology (Lausanne, Switzerland) 11:582658; Zhuang, Q., Cai, H., Cao, O., Li, Z., Liu. S., and Ming, Y. 2020. “Tolerogenic Dendritic Cells: The Pearl of Immunotherapy in Organ Transplantation,” Online, Frontiers in Immunology (Lausanne, Switzerland) 11:552988; Ahad. A., Stevanin, M., Smita. S., Mishra, G. P., Gupta, D., and 6 others 2019. “NCoRI [nuclear receptor co-repressor 1]: Putting the Brakes on the Dendritic Cell Immune Tolerance,” iScience 19:996-1011; Fucikova, J., Palova-Jelinkova, L., Bartunkova, J., and Spisek, R. 2019. “Induction of Tolerance and Immunity by Dendritic Cells: Mechanisms and Clinical Applications,” Online, Frontiers in Immunology (Lausanne, Switzerland) 10:2393; Thomson, A. W., Metes, D. M., Ezzelarab, M. B., and Raich-Regue, D. 2019. Op cit., section 2a, entitled Background. General; Ezzelarab, M. B., Lu, L., Shufesky, W. F., Morelli. A. E., and Thomson, A. W. 2018. “Donor-derived Regulatory Dendritic Cell Infusion Maintains Donor-reactive CD4+ CTLA4hi T Cells in Non-human Primate Renal Allograft Recipients Treated with CD28 Co-stimulation Blockade,” Online, Frontiers in Immunology (Lausanne, Switzerland) 9:250; Hasegawa, H. and Matsumoto, T. 2018. “Mechanisms of Tolerance Induction by Dendritic Cells in Vivo,” Online, Frontiers in Immunology (Lausanne, Switzerland) 9:350; Marin, E., Cuturi, M. C., and Moreau, A. 2018. “Tolerogenic Dendritic Cells in Solid Organ Transplantation: Where Do We Stand?,” Online, Frontiers in Immunology (Lausanne, Switzerland) 9:274; Rosen, S., Harris, P. E., and Hardy, M. A. 2018. “State of the Art: Role of the Dendritic Cell in Induction of Allograft Tolerance,” Online, Transplantation 102(10):1603-1613; Domogalla, M. P., Rostan, P. V., Raker, V. K., and Steinbrink, K. 2017. “Tolerance through Education: How Tolerogenic Dendritic Cells Shape Immunity,” Online, Frontiers in Immunology (Lausanne, Switzerland) 8:1764; Moreau, A., Alliot-Licht, B., Cuturi, M. C., and Blancho, G. 2017. “Tolerogenic Dendritic Cell Therapy in Organ Transplantation,” Online, Transplant International 30(8):754-764; Obregon, C., Kumar, R., Pascual, M. A., Vassalli, G., and Golshayan, D. 2017. “Update on Dendritic Cell-induced Immunological and Clinical Tolerance,” Online, Frontiers in Immunology (Lausanne, Switzerland) 8:1514; Scalea, J. R., Tomita, Y., Lindholm, C. R., and Burlingham, W. 2016. “Transplantation Tolerance Induction: Cell Therapies and Their Mechanisms,” Online, Frontiers in Immunology (Lausanne, Switzerland) 7:87; Ezzelarab, M. and Thomson, A. W. 2011. Tolerogenic Dendritic Cells and Their Role in Transplantation,” Online, Seminars in Immunology 23(4):252-263; additional reference cited above in section 2a, entitled Background. General).

d. Coinjection or coinfusion of bone marrow cells or bone-marrow, umbilical cord mesenchymal stromal, or liver-derived stem cells (see, for example, ten Brinke, A., Martinez-Llordella, M., Cools, N., Hilkens, C. M. U., van Ham, S. M., and 5 others 2019. “Ways Forward for Tolerance-inducing Cellular Therapies-A FACTT [action to Focus and Accelerate Cell Based Tolerance-inducing Therapies] Perspective,” Online, Frontiers in Immunology (Lausanne, Switzerland) 10:181; Reinders. M. E. J., van Kooten, C., Rabelink, T. J., and de Fijter, J. W. 2018. “Mesenchymal Stromal Cell Therapy for Solid Organ Transplantation,” Transplantation 102(1):35-43; Locatelli, F., Algeri, M., Trevisan, V., and Bertaina, A. 2017. “Remestemcel-L for the Treatment of Graft Versus Host Disease,” Expert Review of Clinical Immunology 13(1):43-56; Tang, Q. and Vincenti, F. 2017. “Transplant Trials with Tregs [regulatory suppressor T cells]: Perils and Promises,” Journal of Clinical Investigation 127(7):2505-2512; Plock, J. A., Schnider, J. T., Schweizer, R., Zhang. W., Tsuji, W., and 5 others 2017. “The Influence of Timing and Frequency of Adipose-derived Mesenchymal Stem Cell Therapy on Immunomodulation Outcomes after Vascularized Composite Allotransplantation,” Transplantation 101(1):e1-e11; Lee, C. W., Huang, W. C., Huang, H. D., Huang, Y. H., Ho, J. H., Yang, M. H., Yang, V. W., and Lee, O. K. 2017. “DNA Methyltransferases Modulate Hepatogenic Lineage Plasticity of Mesenchymal Stromal Cells,” Stem Cell Reports 2017 9(1):247-263; Heyes. R., Iarocci, A., Tchoukalova, Y., and Lott, D. G. 2016. “Immunomodulatory Role of Mesenchymal Stem Cell Therapy in Vascularized Composite Allotransplantation,” Journal of Transplantation 2016:6951693; Wang. L., Gu, Z., Zhao, X., Yang, N., Wang. F., and 10 others 2016. “Extracellular Vesicles Released from Human Umbilical Cord-derived Mesenchymal Stromal Cells Prevent Life-Threatening Acute Graft-Versus-Host Disease in a Mouse Model of Allogeneic Hematopoietic Stem Cell Transplantation,” Stem Cells and Development 2016 25(24):1874-1883; Urschel, S., Ryan, L. A., M Larsen, I., Biffis, K., Dijke, I. E., and West. L. J. 2016. “Development of B-cell Memory in Early Childhood and the Impact on Antigen-specific Tolerance after Heart Transplantation,” Journal of Heart and Lung Transplantation (New York, New York) 35(4):491-499; Kobayashi, E. 2016, Op cit.; Haarer, J., Johnson, C. L., Soeder, Y., and Dahlke, M. H. 2015. “Caveats of Mesenchymal Stem Cell Therapy in Solid Organ Transplantation,” Transplantation International 28(1):1-9; Takebe, T., Enomura, M., Yoshizawa, E., Kimura, M., Koike, H., and 8 others 2015. “Vascularized and Complex Organ Buds from Diverse Tissues via Mesenchymal Cell-driven Condensation,” Cell Stem Cell 16(5):556-565; Zhao, K., Lou, R., Huang, F., Peng. Y., Jiang, Z., and 11 others 2015. “Immunomodulation Effects of Mesenchymal Stromal Cells on Acute Graft-versus-host Disease after Hematopoietic Stem Cell Transplantation,” Biology of Blood and Marrow Transplantation 21(1):97-104; Im, K. I., Park, M. J., Kim, N., Lim, J. Y., Park, H. S., and 6 others 2014. Op cit. above in section 2b(1)(b)(b2), entitled Prenatal Compound Vascular Bypass, or Sudden Switched-organ Transplantation of the Heart; Holtick, U., Albrecht, M., Chemnitz, J. M., Theurich, S., Skoetz, N., Scheid, C., and von Bergwelt-Baildon, M. 2014. “Bone Marrow versus Peripheral Blood Allogeneic Haematopoietic Stem Cell Transplantation for Haematological Malignancies in Adults,” Cochrane Database of Systematic Reviews(4):CD010189; Iwai, S., Sakonju, I., Okano. S., Teratani, T., Kasahara, N., and 3 others 2014. “Impact of Ex Vivo Administration of Mesenchymal Stem Cells on the Function of Kidney Grafts from Cardiac Death Donors in Rat” Transplantation Proceedings 46(5):1578-1584; Fox, I. J. 2014. “Hepatocyte Transplantation,” Gastroenterology and Hepatology (New York) 10(9):594-596; Michael, M., Shimoni, A., and Nagler, A. 2013. “Regulatory T Cells in Allogeneic Stem Cell Transplantation,” Clinical and Developmental Immunology 2013:608951; Jindra, P. T., Tripathi, S., Tian, C., Iacomini, J., and Bagley, J. 2013. “Tolerance to MHC [major histocompatibility complex] Class II Disparate Allografts through Genetic Modification of Bone Marrow,” Gene Therapy 20(5):478-486; Pilat, N., Hock, K., and Wekerle. T. 2012. Op cit. above in section 2b(1)(b)(b2), entitled Prenatal Compound Vascular Bypass, or Sudden Switched-organ Transplantation of the Heart; Zhang, H., Chen, J., and Que. W. 2012. “Allogeneic Peripheral Blood Stem Cell and Bone Marrow Transplantation for Hematologic Malignancies: Meta-analysis of Randomized Controlled Trials,” Leukemia Research 36(4):431-437; Edinger, M. and Hoffmann, P. 2011. “Regulatory T Cells in Stem Cell Transplantation: Strategies and First Clinical Experiences,” Current Opinion in Immunology 23(5):679-684; Itakura, S., Asari, S., Rawson, J., Ito, T., Todorov, I., and 4 others 2007. “Mesenchymal Stem Cells Facilitate the Induction of Mixed Hematopoietic Chimerism and Islet Allograft Tolerance without GVHD [graft versus host disease] in the Rat,” American Journal of Transplantation 7(2):336-346; Slavin, S. 2002, Op cit.). Murid models such as in the paper by Itakura et al. 2007 just above are not necessarily applicable to humans but are likely to be so in veterinary practice.

In this connection, the use of ductus side-entry jackets with a magnetic layer can be instrumental in the use of magnetically susceptible nanoparticles (Silva, L. H., Cruz, F. F., Morales, M. M., Weiss. D. J., and Rocco, P. R. 2017. “Magnetic Targeting as a Strategy to Enhance Therapeutic Effects of Mesenchymal Stromal Cells,” Stem Cell Research and Therapy 8(1):58; Strehl. C., Maurizi, L., Gaber. T., Hoff. P., Broschard, T., Poole, A. R., Hofmann, H., and Buttgereit, F. 2016. “Modification of the Surface of Superparamagnetic Iron Oxide Nanoparticles to Enable Their Safe Application in Humans,” International Journal of Nanomedicine 11:5883-5896; Ma, J., Mercer, R. R., Barger, M., Schwegler-Berry, D., Cohen, J. M., Demokritou, P., and Castranova, V. 2015. “Effects of Amorphous Silica Coating on Cerium Oxide Nanoparticles Induced Pulmonary Responses,” Toxicology and Applied Pharmacology 288(1):63-73; Schulze, F., Dienelt, A., Geissler, S., Zaslansky, P., Schoon, J., and 9 others 2014. “Amino-polyvinyl Alcohol Coated Superparamagnetic Iron Oxide Nanoparticles are Suitable for Monitoring of Human Mesenchymal Stromal Cells in Vivo,” Small (Weinheim an der Bergstrasse, Germany) 10(21):4340-4351).

Moreover, amino-functionalized amino-polyvinyl alcohol coated superparamagnetic iron oxide nanoparticles not only impart greater safety (Strehl. C., Maurizi, L., Gaber, T., Hoff, P., et al. 2016. Op cit.;

Ma. J., Mercer. R. R., et al. 2015, Op cit.; Schulze, F., Dienelt. A., Geissler, S., Zaslansky, P., et al. 2014. Op cit.) as compared with uncoated nanoparticles (which can be extracted from the bloodstream by means of one or more extraction impasse-jackets as described and illustrated in U.S. Pat. No. 11,389,171, said to induce thrombotic and toxic consequences (Yarjanli, Z., Ghaedi, K., Esmaeili. A., Rahgozar, S., and Zarrabi. A. 2017. “Iron Oxide Nanoparticles May [Cause] Damage to the Neural Tissue Through Iron Accumulation, Oxidative Stress, and Protein Aggregation,” Online, BioMed Central Neuroscience 18(1):51; Nemmar, A., Al-Salam, S., Beegam, S., Yuvaraju, P., and Ali, B. H. 2017. “The Acute Pulmonary and Thrombotic Effects of Cerium Oxide Nanoparticles after Intratracheal Instillation in Mice,” International Journal of Nanomedicine 12:2913-2922; Engin, A. B., Nikitovic, D., Neagu, M., Henrich-Noack, P., Docea. A. O., Shtilman, M. I., Golokhvast, K., and Tsatsakis, A. M. 2017. “Mechanistic Understanding of Nanoparticles' Interactions with Extracellular Matrix: The Cell and Immune System,” Particle and Fibre Toxicology 14(1):22; Nemmar, A., Beegam, S., Yuvaraju, P., Yasin, J., Tariq, S., Attoub. S., and Ali, B. H. 2016. “Ultrasmall Superparamagnetic Iron Oxide Nanoparticles Acutely Promote Thrombosis and Cardiac Oxidative Stress and DNA Damage in Mice,” Particle and Fibre Toxicology 13(1):22) but offer a new capability for evaluating the rejection status of transplant tissue (see, for example, Strehl. C., Schellmann, S., Maurizi, L., Hofmann-Amtenbrink, M., Haupl. T., Hofmann, H., Buttgereit, F., and Gaber. T. 2016. “Effects of PVA-coated Nanoparticles on Human T Helper Cell Activity,” Toxicology Letters 245:52-58; Strehl. C., Gaber, T., Maurizi, L., Hahne. M., Rauch, R., and 6 others 2015. “Effects of PVA Coated Nanoparticles on Human Immune Cells,” International Journal of Nanomedicine 10:3429-3445; Vermeij. E. A., Koenders, M. I., Bennink, M. B., Crowe, L. A., Maurizi. L., and 5 others 2015. “The In-vivo Use of Superparamagnetic Iron Oxide Nanoparticles to Detect Inflammation Elicits a Cytokine Response but Does Not Aggravate Experimental Arthritis,” Online, PLoS [Public Library of Science] One 10(5):e0126687).

Bone marrow heme oxygenase-1 modified mesenchymal stem cells have also been used following liver transplantation and found to improve hepatic sinusoid microcirculation (Yang, L., Shen, Z. Y., Wang. R. R., Yin, M. L., Zheng. W. P., Wu. B., Liu. T., and Song, H. L. 2017. “Effects of Heme Oxygenase-1-modified Bone Marrow Mesenchymal Stem Cells on Microcirculation and Energy Metabolism following Liver Transplantation,” World Journal of Gastroenterology 23(19):3449-3467; Shen, Z. Y., Wu. B., Liu, T., Yang, Y., Yin, M. L., Zheng. W. P., Zhang, B. Y., and Song. H. L. 2017. “Immunomodulatory Effects of Bone Marrow Mesenchymal Stem Cells Overexpressing Heme Oxygenase-1: Protective Effects on Acute Rejection Following Reduced-size Liver Transplantation in a Rat Model,” Cellular Immunology 313:10-24; Yang, Y., Song, H. L., Zhang, W., Wu, B. J., Fu, N. N., Dong. C., and Shen, Z. Y. 2016. “Here Oxygenase-1-transduced Bone Marrow Mesenchymal Stem Cells in Reducing Acute Rejection and Improving Small Bowel Transplantation Outcomes in Rats,” Stem Cell Research and Therapy 7(1):164). However, findings based upon murid models do not necessarily translate to effective therapeutic measures in man or do not without modification.

e. Mesenchymal and hematopoietic stem cells (see, for example, Wang. L., Zhang, H., Guan, L., Zhao, S., Gu, Z., and 9 others 2016. “Mesenchymal Stem Cells Provide Prophylaxis against Acute Graft-versus-host Disease following Allogeneic Hematopoietic Stem Cell Transplantation: A Meta-analysis of Animal Models,” Oncotarget 7(38):61764-61774; Kallekleiv. M., Larun, L., Bruserud, O., and Hatfield, K. J. 2016. “Co-transplantation of Multipotent Mesenchymal Stromal Cells in Allogeneic Hematopoietic Stem Cell Transplantation: A Systematic Review and Meta-analysis,” Cytotherapy 18(2):172-185; Chen, X., Wang, C., Yin, J., Xu. J., Wei. J., and Zhang, Y. 2015. “Efficacy of Mesenchymal Stem Cell Therapy for Steroid-Refractory Acute Graft-Versus-Host Disease following Allogeneic Hematopoietic Stem Cell Transplantation: A Systematic Review and Meta-Analysis,” Online, PLoS [Public Library of Science] One 10(8):e0136991; Im, K. I., Park, M. J., Kim, N., Lim, J. Y., Park, H. S., and 6 others 2014, Op cit. above in this section; Asari, S., Itakura, S., Rawson, J., Ito. T., Todorov, I., and 5 others 2011. “Mesenchymal Stem Cells Facilitate Mixed Hematopoietic Chimerism Induction and Prevent Onset of Diabetes in Nonobese Diabetic Mice,” Pancreas 2011 40(6):846-854; Serreze, D. V., Osborne, M. A., Chen, Y. G., Chapman, H. D., Pearson, T., Brehm, M. A., and Greiner, D. L. 2006. “Partial Versus Full Allogeneic Hemopoietic Chimerization is a Preferential Means to Inhibit Type 1 Diabetes as the Latter Induces Generalized Immunosuppression,” Journal of Immunology (Baltimore, Maryland) 177(10):6675-6684; Wekerle. T. and Sykes, M. 1999. “Mixed Chimerism as an Approach for the Induction of Transplantation Tolerance,” Transplantation 68(4):459-467).

f. Thymus gland grafts (usually by implanting allogeneic thymus tissue into the thigh of the baby undergone to correct congenital athymia in DiGeorge syndrome neonates), thymocyte, or thymus derived cell surface molecule leukapheresis and infusion (see, for example, Howley, E., Davies, E. G., and Kreins, A. Y. 2023. “Congenital Athymia: Unmet Needs and Practical Guidance,” Online, Therapeutics and Clinical Risk Management (Auckland, New Zealand) 19:239-254; Collins, C. Sharpe, E., Silber, A., Kulke, S., and Hsieh, E. W. Y. 2021. “Congenital Athymia: Genetic Etiologies, Clinical Manifestations, Diagnosis, and Treatment,” Online, Journal of Clinical Immunology (Amsterdam, North Holland, Netherlands) 41(5):881-895; Klocperk, A., Paračkova, Z., Bloomfield, M., Rataj. M., Pokomy, J., and 3 others 2018. “Follicular Helper T Cells in DiGeorge Syndrome,” Online, Frontiers in Immunology (Lausanne, Switzerland) 9:1730; Hall, B. M. 2016. “CD4+CD25 [cluster of differentiation]+T Regulatory Cells in Transplantation Tolerance: 25 Years On,” Transplantation 100(12):2533-2547; Gołąb. K., Grose. R., Trzonkowski, P., Wickrema, A., Tibudan, M., and 6 others 2016. “Utilization of Leukapheresis and CD4 Positive Selection in Treg Isolation and the Ex-vivo Expansion for a Clinical Application in Transplantation and Autoimmune Disorders,” Oncotarget 7(48):79474-79484; Hall. B. M., Robinson, C. M., Plain, K. M., Verma, N. D., Tran, G. T., and 4 others 2017. “Changes in Reactivity in Vitro of CD4+CD25+ and CD4+CD25-T Cell Subsets in Transplant Tolerance,” Online, Frontiers in Immunology (Lausanne, Switzerland) 8:994; Hall, B. M., Tran, G. T., Robinson, C. M., and Hodgkinson, S. J. 2015. “Induction of Antigen Specific CD4(+) CD25(+) Foxp3(+) T Regulatory Cells from Naive Natural Thymic Derived T Regulatory Cells,” International Immunopharmacology 28(2):875-886; Theil, A., Tuve. S., Oelschlägel. U., Maiwald, A., Döhler, D., and 11 others 2015. “Adoptive Transfer of Allogeneic Regulatory T Cells into Patients with Chronic Graft-versus-host Disease,” Cytotherapy 17(4):473-486; Hori. T., Kuribayashi, K., Saito, K., Wang, L., Torii, M., Uemoto, S., Iida, T., Yagi, S., and Kato, T. 2015. “Ultraviolet-induced Alloantigen-specific Immunosuppression in Transplant Immunity,” Online, World Journal of Transplantation (Pleasanton, California) 5(1):11-18; Hall, B. M. 2015. “T Cells: Soldiers and Spies—The Surveillance and Control of Effector T Cells by Regulatory T Cells,” Clinical Journal of the American Society of Nephrology 10(11):2050-2064; Glowacki, A. J., Gottardi, R., Yoshizawa, S., Cavalla, F., Garlet, G. P., Sfeir, C., and Little, S. R. 2015. “Strategies to Direct the Enrichment, Expansion, and Recruitment of Regulatory Cells for the Treatment of Disease,” Annals of Biomedical Engineering 43(3):593-602; Plain, K. M., Verma, N. D., Tran, G. T., Nomura, M., Boyd, R., Robinson, C. M., Hodgkinson, S. J., and Hall, B. M. 2013. “Cytokines Affecting CD4(+) T Regulatory Cells in Transplant Tolerance. Interleukin-4 Does Not Maintain Alloantigen Specific CD4(+) CD25(+) Treg,” Transplant Immunology 29(1-4):51-59; Davies, E. G. 2013. “Immunodeficiency in DiGeorge Syndrome and Options for Treating Cases with Complete Athymia,” Online, Frontiers in Immunology (Lausanne, Switzerland) 4:322; Markert, M. L., Devlin, B. H., and Mccarthy, E. A. 2010. “Thymus Transplantation,” Clinical Immunology (Orlando, Florida) 135(2):236-246; Hall. B. M., Tran, G., and Hodgkinson, S. J. 2009. “Alloantigen Specific T Regulatory Cells in Transplant Tolerance,” International Immunopharmacology 9(5):570-574; Verma, N. D., Plain, K. M., Nomura, M., Tran, G. T., Robinson, C., Boyd, R., Hodgkinson, S. J., and Hall, B. M. 2009. “CD4+CD25+ T Cells Alloactivated Ex Vivo by IL-2 or IL-4 Become Potent Alloantigen-specific Inhibitors of Rejection with Different Phenotypes, Suggesting Separate Pathways of Activation by Th1 and Th2 Responses,” Blood 113(2): 479-487; Nagahara, K., Nishimura, E., and Sakaguchi, S. 2007. “Induction of Tolerance by Adoptive Transfer of Treg Cells,” Methods in Molecular Biology 380: 431-442; Serreze, D. V., Osborne, M. A., Chen, Y. G., Chapman, H. D., Pearson, T., Brehm, M. A., and Greiner, D. L. 2006, Op cit.; Biggar, W. D., Park, B. Y., Stutman, O., Gail-Peczalska, K., and Good, R. A. 1975. “Fetal Thymus Transplantation: Experimental and Clinical Observations,” Birth Defects Original Article Series 11(1):361-366).

g. Liver grafts or hepatocytes (see, for example, Lee, C. W., Huang, W. C., Huang, H. D., Huang, Y. H., Ho, J. H., Yang, M. H., Yang, V. W., and Lee, O. K. 2017, Op cit.; Tarlow, B. D., Pelz, C., Naugler, W. E., Wakefield, L., Wilson, E. M., Finegold, M. J., and Grompe. M. 2014. “Bipotential Adult Liver Progenitors are Derived from Chronically Injured Mature Hepatocytes,” Cell Stem Cell 15(5):605-618; Starzl. T. E., Demetris, A. J., Murase, N., Trucco, M., Thomson, A. W., Rao, A. S., and Fung, J. J. 1997. “Chimerism after Organ Transplantation,” Current Opinion in Nephrology and Hypertension 6(3):292-298; Starzl. T. E. 1997. “Clinical and Basic Scientific Implications of Cell Migration and Microchimerism after Organ Transplantation,” Artificial Organs 21(11):1154-1155; Starzl, T. E., Demetris, A. J., Murase, N., Trucco, M., Thomson, A. W., and Rao, A. S. 1995. “The Changing Immunology of Organ Transplantation,” Hospital Practice 30(10):31-34, 37-42; Rao, A. S., Thomson, A. W., Shapiro. R., and Starzl. T. E. 1994. “Chimerism after Whole Organ Transplantation: Its Relationship to Graft Rejection and Tolerance Induction,” Current Opinion in Nephrology and Hypertension 3(6):589-595) or

h. Spleen transplantation in the context of multiple organ transplantation is addressed above in section 2a(2), entitled Inventive Content-pertinent Fetal Diagnostics. Spleen allografts or splenocyte infusions (see, for example, Jansson, K., Dreckmann, K., Sommer, W., Avsar, M., Salman, J., and 10 others 2017. Op cit.; Sommer, W., Buechler, G., Jansson, K., Avsar, M., Knofel, A. K., and 10 others 2017. “Irradiation Before and Donor Splenocyte Infusion Immediately after Transplantation Induce Tolerance to Lung, but Not Heart Allografts in Miniature Swine,” Transplantation International 30(4):420-431; Avsar. M., Jansson, K., Sommer, W., Kruse. B., Thissen, S., and 16 others 2016. “Augmentation of Transient Donor Cell Chimerism and Alloantigen-specific Regulation of Lung Transplants in Miniature Swine,” American Journal of Transplantation 16(5):1371-1382; Hara, H., Lin, Y. J., Tai, H. C., Ezzelarab, M., Quader, M. A., Houser, S. L., Nakao, A., and Cooper, D. K. C. 2014. “Hematopoietic Chimerism following Allotransplantation of the Spleen, Splenocytes or Kidney in Pigs,” Transplantation Immunology 31(3):125-133; Yamazaki, S., Kanamoto. A., and Takayama, T. 2013. “Donor-specific Tolerance Induction in Organ Transplantation via Mixed Splenocytes Chimerism,” Clinical and Experimental Immunology 173(2):173-178; Kato, T., Tzakis, A. G., Selvaggi, G., Gaynor, J. J., Takahashi, H., and 10 others 2007. “Transplantation of the Spleen: Effect of Splenic Allograft in Human Multivisceral Transplantation,” Annals of Surgery (Philadelphia, Pennsylvania) 246(3):436-446, Op cit. above in section 2a(2), entitled Inventive Content-pertinent Fetal Diagnostics; Kato, T., Kleiner, G., David, A., Selvaggi, G., Nishida, S., Madariaga, J., Thompson, J., Ruiz, P., and Tzakis, A. 2006. “Inclusion of Spleen in Pediatric Multivisceral Transplantation,” Transplantation Proceedings 38(6):1709-1710. Op cit. above in section 2a(2), entitled Inventive Content-pertinent Fetal Diagnostics; Dor, F. J. and Cooper, D. K. C. 2006. “Immunologic Benefits of Spleen Transplantation in the Absence of Graft-versus- host Disease,” Annals of Surgery (Philadelphia, Pennsylvania) 243(5):710-711; Dor, F. J., Tseng, Y. L., Kuwaki, K., Gollackner, B., Ramirez, M. L., and 8 others 2005. “Immunological Unresponsiveness in Chimeric Miniature Swine following MHC-mismatched Spleen Transplantation,” Transplantation 80(12):1791-1804; Nakafusa, Y., Goss, J. A., Mohanakumar. T., and Flye, M. W. 1993. “Induction of Donor-specific Tolerance to Cardiac but Not Skin or Renal Allografts by Intrathymic Injection of Splenocyte Alloantigen,” Transplantation 55(4):877-882).

i. Thymic grafting is often included to promote tolerance induction in the xenotransplantation of other solid organs (see, for example, Yamada, K., Ariyoshi, Y., Pomposelli. T., and Sekijima, M. 2020. “Co-transplantation of Vascularized Thymic Graft with Kidney in Pig-to-Nonhuman Primates for the Induction of Tolerance across Xenogeneic Barriers,” Online, Methods in Molecular Biology (Totowa, New Jersey) 2110:151-171; Yamada, K. and Scalea. J. 2012. “Thymic Transplantation in Pig-to-Nonhuman Primates for the Induction of Tolerance across Xenogeneic Barriers,” Methods in Molecular Biology (Totowa, New Jersey) 885:191-212; Barth R N. Yamamoto S, LaMattina J C, Kumagai N. Kitamura H, and 7 others 2003. “Xenogeneic Thymokidney and Thymic Tissue Transplantation in a Pig-to-Baboon Model: I. Evidence for Pig-specific T-cell Unresponsiveness,” Online, Transplantation (Hagerstown, Maryland) 75(10):1615-1624).

Additional drugs posing the possibility of numerous drug-drug and drug-food interactions and side effects, the release of all drugs whether or not directed to the native organ demands that the transplant be taken into account. Broadly, any drugs for which a clear purpose and benefit cannot be specified but which would introduce additional contingency, complexity, and confusion, should be eliminated, or deprescribed. The automatic system is therefore freed to concentrate on the targeted release of graft organ supportive antimicrobials and immunosuppressants.

The potential complications of standard procedural components when used in adults such as extracorporeal life support can be more harmful in a neonate than an adult. The postprocedural sequelae associated with cardiopulmonary bypass or extracorporeal life support, for example, include cognitive dysfunction and kidney damage (see, for example, Adams, L. E., Mukherjee. R., and Hollinger, L. E. 2022. “Complication Accrual Impacts Pediatric Extracorporeal Life Support Mortality,” Pediatric Surgery International 38(7):985-991; Zhang, P., Tong, Y., Liu, J., Guo, S., Jin, Y., and 4 others 2022. “The Lower Threshold of Hypothermic Oxygen Delivery to Prevent Neonatal Acute Kidney Injury,” Pediatric Research 91(7):1741-1747; Said, A. S., Guilliams, K. P., and Bembea, M. M. 2020. “Neurological Monitoring and Complications of Pediatric Extracorporeal Membrane Oxygenation Support,” Online, Pediatric Neurology 108:31-39; other reference above in section 2a, entitled Background. General, and below in this section).

To accommodate interdependent comorbid consequences may require that certain drugs be not simply withdrawn, and if not spontaneously voided quickly enough, recovered or neutralized, in which case the patient is treated by apheresis and reversal agents as necessary, evaluated for new drugs, or some combination of withdrawal and replacement, and fitted with a fully implanted ambulatory prosthetic disorder response system prior to discharge as addressed in U.S. Pat. No. 11,759,186, entitled Ductus Side-entry Jackets and Prosthetic Disorder Response Systems.

Once implanted, drugs are released by the automatic system, those with a more complicated schedule of administration receiving higher priority. With prescription regimen-adherent patients, leaving drugs following the simplest scheduling to be taken orally or by intramuscular self injection reduces the cost and complexity of the prescription-program and the need for nonessential implanted drug reservoirs.

During compound vascular bypass solid organ transplantation, either or both the donor and recipient may require an inotrope or a transluminally delivered assist device to impart sufficient pressure to the blood in the lines connecting the recipient to the donor. The availability of a suitable graft organ confirmed, drugs administered prior to transplantation are reevaluated to eliminate those not needed by or potentially harmful to the new heart. Drugs directed at comorbidities are generally continued, especially where these were substantially independent of the impaired heart and not likely to subside with the new heart. In an adult, especially geriatric, where typically, comorbidities both dependent and substantially independent of the heart failure will be present, heart transplantation will usually be necessitated by congestive heart failure which had progressed over a number of years.

Since different treatments have an onset of action, that is, take effect over a wide range of lead times, preparation varies according to the time available (see, for example, Lim, H. S. 2017. “The Effect of Impella CP on Cardiopulmonary Physiology during Venoarterial Extracorporeal Membrane Oxygenation Support,” Artificial Organs June 7 [in press]: Moazzami, K., Dolmatova, E. V., Cocke. T. P., Elmann, E., Vaidya. P., and 3 others 2017. “Left Ventricular Mechanical Support with the Impella [emporary ventricular assist device manufactured by Abiomed, Inc., Danvers, Massachusetts] during Extracorporeal Membrane Oxygenation,” Journal of Tehran Heart Center 12(1):11-14; Curley, M., Liebers. J., and Maynard, R. 2017. “Continuous Intravenous Milrinone Therapy in Pediatric Outpatients,” Journal of Infusion Nursing 40(2):92-96; Wen, S., Ning. J., Collins, S., and Berry, D. 2017. “A Response-adaptive Design of Initial Therapy for Emergency Department Patients with Heart Failure,” Contemporary Clinical Trials 52:46-53; Ishihara, S., Gayat, E., Sato, N., Arrigo, M., Laribi, S., and 7 others 2016. “Similar Hemodynamic Decongestion with Vasodilators and Inotropes: Systematic Review, Meta-analysis, and Meta-regression of 35 Studies on Acute Heart Failure,” Clinical Research in Cardiology 105(12):971-980; Jaiswal. A., Nguyen, V. Q., Le Jemtel. T. H., and Ferdinand, K. C. 2016. “Novel Role of Phosphodiesterase Inhibitors in the Management of End-stage Heart Failure,” World Journal of Cardiology 8(7):401-412; Krishnamurthy, Y., Cooper, L. B., Parikh, K. S., Felker, G. M., Milano, C. A., and 3 others 2016. “Pulmonary Hypertension in the Era of Mechanical Circulatory Support,” American Society for Artificial Internal Organs J 62(5):505-512; Rosenkranz, S., Gibbs, J. S., Wachter, R., De Marco. T., Vonk-Noordegraaf. A., and Vachiery, J. L. 2016. “Left Ventricular Heart Failure and Pulmonary Hypertension,” European Heart Journal 37(12):942-954; Liu, L. C., Dorhout, B., van der Meer, P., Teerlink, J. R., and Voors. A. A. 2016. “Omecamtiv Mecarbil: A New Cardiac Myosin Activator for the Treatment of Heart Failure,” Expert Opinion on Investigational Drugs 25(1):117-127; Pozzi, M., Giraud, R., Tozzi, P., Bendjelid, K., Robin, J., Meyer, P., Obadia, J. F., and Banfi. C. 2015. “Long-term Continuous-flow Left Ventricular Assist Devices (LVAD) as Bridge to Heart Transplantation,” Journal of Thoracic Disease 7(3):532-542; Kapur, N. K. and Esposito, M. 2015. “Hemodynamic Support with Percutaneous Devices in Patients with Heart Failure,” Heart Failure Clinics 11(2):215-230; Tariq. S. and Aronow, W. S. 2015. “Use of Inotropic Agents in Treatment of Systolic Heart Failure,” Online, MDPI [Multidisciplinary Digital Publishing Institute] International Journal of Molecular Sciences (Basel, Switzerland) 16(12):29060-29068; Birnbaum, B. F., Simpson, K. E., Boschert, T. A., Zheng, J., Wallendorf. M. J., Schechtman, K., and Canter, C. E. 2015. “Intravenous Home Inotropic Use is Safe in Pediatric Patients Awaiting Transplantation,” Circulation. Heart Failure 8(1):64-70; Hashim, T., Sanam, K., Revilla-Martinez, M., Morgan, C. J., Tallaj. J. A., and 4 others 2015. “Clinical Characteristics and Outcomes of Intravenous Inotropic Therapy in Advanced Heart Failure,” Circulation. Heart Failure 8(5):880-886; den Uil, C. A. and Brugts, J. J. 2015. “Impact of Intravenous Nitroglycerin in the Management of Acute Decompensated Heart Failure,” Current Heart Failure Reports 12(1):87-93; Hill, N. S., Preston, I. R., and Roberts, K. E. 2015. “Inhaled Therapies for Pulmonary Hypertension,” Respiratory Care 60(6):794-805; Gerges, M., Gerges, C., Pistritto, A. M., Lang, M. B., Trip, P., Jakowitsch, J., Binder, T., and Lang, I. M. 2015. “Pulmonary Hypertension in Heart Failure. Epidemiology, Right Ventricular Function, and Survival,” American Journal of Respiratory and Critical Care Medicine 192(10):1234-1246; Holley, C. T., Harvey, L., and John, R. 2014. “Left Ventricular Assist Devices as a Bridge to Cardiac Transplantation,” Journal of Thoracic Disease 6(8):1110-1119; Mortara, A., Oliva, F., Metra, M., Carbonieri, E., Di Lenarda, A., 7 others, and 138 collaborators 2014. Treatment with Inotropes and Related Prognosis in Acute Heart Failure: Contemporary Data from the Italian Network on Heart Failure (IN-HF) Outcome Registry,” Journal of Heart and Lung Transplantation (New York, New York) 33(10):1056-1065; Nagy, L., Pollesello, P., and Papp, Z. 2014. “Inotropes and Inodilators for Acute Heart Failure: Sarcomere Active Drugs in Focus,” Journal of Cardiovascular Pharmacology 64(3):199-208; Kalogeropoulos, A. P., Marti, C. N., Georgiopoulou, V. V., and Butler, J. 2014. “Inotrope Use and Outcomes among Patients Hospitalized for Heart Failure: Impact of Systolic Blood Pressure, Cardiac Index, and Etiology,” Journal of Cardiac Failure 20(8):593-601; Guglin, M. and Kaufman, M. 2014. “Inotropes Do Not Increase Mortality in Advanced Heart Failure,” International Journal of General Medicine 7:237-251; Garg, V. and Frishman, W. H. 2013. “A New Approach to Inotropic Therapy in the Treatment of Heart Failure: Cardiac Myosin Activators in Treatment of HF,” Cardiology in Review 21(3):155-159; Georgiopoulou, V. V., Kalogeropoulos, A. P., Borlaug, B. A., Gheorghiade, M., and Butler, J. 2013. “Left Ventricular Dysfunction with Pulmonary Hypertension: Part 1: Epidemiology, Pathophysiology, and Definitions,” Circulation. Heart Failure 6(2):344-354; Kalogeropoulos, A. P., Georgiopoulou, V. V., Borlaug, B. A., Gheorghiade, M., and Butler, J. 2013. “Left Ventricular Dysfunction with Pulmonary Hypertension: Part 2: Prognosis, Noninvasive Evaluation, Treatment, and Future Research,” Circulation. Heart Failure 6(3):584-593; Vachiery, J. L., Adir, Y., Barbera, J. A., Champion, H., Coghlan, J. G., and 10 others 2013. “Pulmonary Hypertension Due to Left Heart Diseases,” Journal of the American College of Cardiology 62(25 Supplement): D100-D108; Amin, A. and Maleki, M. 2012. “Positive Inotropes in Heart Failure: A Review Article,” Heart Asia 4(1):16-22; Schwartzenberg, S., Redfield, M. M., From, A. M., Sorajja, P., Nishimura, R. A., and Borlaug, B. A. 2012. “Effects of Vasodilation in Heart Failure with Preserved or Reduced Ejection Fraction Implications of Distinct Pathophysiologies on Response to Therapy,” Journal of the American College of Cardiology 59(5):442-451; Kawashima, D., Gojo, S., Nishimura, T., Itoda, Y., Kitahori, K., Motomura, N., and 5 others 2011. “Left Ventricular Mechanical Support with Impella Provides More Ventricular Unloading in Heart Failure than Extracorporeal Membrane Oxygenation,” American Society for Artificial Internal Organs Journal 57(3):169-176; Toma, M. and Starling, R. C. 2010. “Inotropic Therapy for End-stage Heart Failure Patients,” Current Treatment Options in Cardiovascular Medicine 12(5):409-419; Berg, A. M., Snell, L., and Mahle, W. T. 2007. “Home Inotropic Therapy in Children,” Journal of Heart and Lung Transplantation (New York, New York) 26(5):453-457; Elkayam, U., Bitar, F., Akhter, M. W., Khan, S., Patrus, S., and Derakhshani, M. 2004. “Intravenous Nitroglycerin in the Treatment of Decompensated Heart Failure: Potential Benefits and Limitations,” Journal of Cardiovascular Pharmacology and Therapy 9(4):227-241; Upadya, S., Lee, F. A., Saldarriaga, C., Verma, S., Sedrakyan, A., Nystrom, K, and Katz, S. D. 2004. “Home Continuous Positive Inotropic Infusion as a Bridge to Cardiac Transplantation in Patients with End-stage Heart Failure,” Journal of Heart and Lung Transplantation (New York, New York) 23(4):466-472; Semigran, M. J., Cockrill, B. A., Kacmarek, R., Thompson, B. T., Zapol, W. M., Dec, G. W., and Fifer, M. A. 1994. “Hemodynamic Effects of Inhaled Nitric Oxide in Heart Failure,” Journal of the American College of Cardiology 24(4):982-988).

For patients on extracorporeal membrane oxygenator support with or without ancillary aids for spontaneous breathing before a sudden switch heart transplant, once circulatory and pulmonary sufficiency following the switch have been confirmed, mechanical support is turned off. In contrast to sudden switch machine support, in metered compound vascular bypass, or sudden switched-organ transplantation, since the interval over which the graft organ is switched from the donor to the recipient is extended, perhaps protracted over a period of days, life support will often be continued well into if not entirely through the switch procedure.

Ductus side-entry and the diversion and choke jackets to be described can be used to target medication to treatment sites in support of conventional procedures and allow new techniques for performing procedures capable of supplanting the conventional. Moreover, because the mainlines joining jackets are provided with accessory channels to allow the direct targeting into the conjoined lumina of medication, ductus side-entry and diversion jackets allow the creation of small caliber shunts and bypasses not susceptible to occlusion due to a buildup of thrombus, crystal, or intractable infection due to the buildup of biofilm.

Using the switch technique, the recipient experiences only slight transient drops in mean arterial blood pressure—while the diversion jackets are placed and when blood flows through the lines from the recipient to the donor heart; otherwise, the recipient is perfused continuously with his own machine damage-spared blood throughout. This procedure thus avoids the pitfalls of the conventional procedure to include nonperfusion while unless transported with the aid of an Organ Care System® (TransMedics, Incorporated, Andover, Massachusetts) console (see above), a disembodied donor heart is kept chilled for hours in ice water, so that the recipient circulates blood damaged as the result of pump support which generates microemboli.

Accordingly, given a good donor-recipient match, immunosuppressant preparation, and the elimination of cross-clamping, cardioplegia, ischemia, and reperfusion injury, sudden circulatory system switching significantly reduces if not eliminates the major causes of adverse sequelae—cardiac allograft vasculopathy, reperfusion injury, postperfusion syndrome, or “pumpheadedness,” and the risk of stroke or other potentially fatal events.

The risk of embolization and inflammation, hence postprocedural ischemic stroke, associated with manipulation of the ascending aorta during placement of the diversion jacket is reduced through the directly targeted delivery through the jacket into the recipient ascending aorta of suitable medication, such as low molecular weight heparin, streptokinase, and triamcinolone, dexamethasone, acetylcholine or similar steroid anti-inflammatory (see, for example, Chivasso, P., Guida, G. A., Fudulu, D., Bruno, V. D., and Marsico, R. 2016, Op cit.; Kowalewski, M., Pawliszak, W., Malvindi, P. G., Bokszanski, M. P., Perlinski, D., and 9 others 2016, Op cit.; Pawliszak, W., Kowalewski, M., Raffa, G. M., Malvindi, P. G., Kowalkowska, M. E., and 4 others 2016. “Cerebrovascular Events after No-touch Off-pump Coronary Artery Bypass Grafting, Conventional Side-clamp Off-pump Coronary Artery Bypass, and Proximal Anastomotic Devices: A Meta-Analysis,” Journal of the American Heart Association (Oxford, England) 5(2) pii: e002802; Moss, E., Puskas, J. D., Thourani, V. H., Kilgo, P., Chen, E. P., and 5 others 2015. “Avoiding Aortic Clamping During Coronary Artery Bypass Grafting Reduces Postoperative Stroke,” Journal of Thoracic and Cardiovascular Surgery 149(1):175-180; Emmert, M. Y., Seifert, B., Wilhelm, M., Grünenfelder, J., Falk, V., and Salzberg, S. P. 2011, Op cit.),

Cross-clamping which completely shuts off the aorta is associated with a risk of stroke and is considerably more disruptive than is the placement of a side-entry jacket on the aorta with pulsatile flow sustained throughout. This a transplant, calcification of the thoracic aorta (“Porcelain aorta”) is not a factor. Neither sudden nor metered switching of the transplant from the circulatory system of the donor to that of the recipient in the same sequence cannot be accomplished with an arrangement of balloons; diversion jackets not only obturate but divert flow, and serve no less to allow the mid and postprocedural direct targeting of medication to the treatment site on a permanent postoperative basis. Balloons cannot do this. No such procedure using balloons is contemplated in the literature in any event.

### Heart Transplantation Options

Switched-organ heart transplantation is potentially more versatile and fundamentally less traumatizing surgically than is conventional heart transplantation, which includes orthotopic replacement of a severely impaired native heart and ‘heterotopic’ transplantation where the ventricles of a second heart are added to that native. Adding ventricles to a previously transplanted, hence denervated replacement heart, is not reported.

In comparison, switched heart transplantation-which eliminates the need to bisect recipient and donor hearts, ischemia, hence, ischemic time and ischemia-reperfusion injury, as well as the need in a mentally balanced adult for general anesthesia, and cardiopulmonary bypass with which the brain might receive only 30 percent of the oxygen it should, possibly along with microscopic air bubbles and debris resulting in postperfusion, or ‘pumphead’ and vasoplegic syndromes (see, for example, Mukaida, H., Matsushita, S., Minami, Y., Sato, G., Usuba, M., and 3 others 2022. “Risk Factors for Postoperative Delirium on Oxygen Delivery-guided Perfusion,” Online, BioMed Central Journal of Cardiothoracic Surgery 17(1):193; Busse, L. W., Barker, N., and Petersen, C. 2020. “Vasoplegic Syndrome following Cardiothoracic Surgery-Review of Pathophysiology and Update of Treatment Options,” Online, Critical Care (London, England) 24(1):36; Mukaida, H., Matsushita, S., Kuwaki, K., Inotani, T., Minami, Y., Saigusa, A., and Amano, A. 2019. “Time-dose Response of Oxygen Delivery during Cardiopulmonary Bypass Predicts Acute Kidney Injury,” Online, Journal of Thoracic and Cardiovascular Surgery 158(2):492-499; Leenders, J., Overdevest, E., van Straten, B., and Golab, H. 2018. “The Influence of Oxygen Deliver during Cardiopulmonary Bypass on Incidence of Delirium in CABG Patients; a Retrospective Study,” Online, Perfusion 33(8):656-662), includes the orthotopic replacement of the native heart, the addition of a second, less than perfect heart to the first without incising either, or the replacement of an end-stage heart with two less than perfect hearts.

In switched-organ double heart transplants, each heart remains separately and automatically targetable with medication such as inotropic, anti-inflammatory, immunosuppressive or antimicrobial, and/or resynchronization therapy. Each heart separate from the other, either could at least for a time support the patient temporarily were the other to cease functioning due to ventricular fibrillation or a sudden arrest, for example. As described below in section 2b(1)(b)(b16), entitled Direct Aortocaval Connection: Unified Entry Arterial and Exit Venous Confluence Conduits for the Heterotopic Connection of a Graft Heart in the Lower Abdomen, the separate serviceability of a second heart is even greater when it is unconnected to that native or its orthotopic replacement but rather independent.

a. The Native Heart is Unsalvageable, and a Good Replacement Heart is Available

Type 1. Orthotopic heart transplantation A sound replacement heart available, an irreparably impaired native heart—one at stage 3 or 4 failure according to the New York Heart Association functional classification—is replaced by switched-organ transplantation. If the need for replacement is urgent, then sudden-switch transplantation with vascular solenoid valves is used. If not, then metered compound vascular bypass, or gradually switched-organ transplantation using vascular servovalves is used. Both sudden and metered switched-organ transplantation transfer the graft directly from the circulatory system of the donor maintained on life support into the circulatory system of the recipient.

While the periprocedural administration of immune tolerance induction medication is recommended with either switched-organ technique, metered transfer affords an additional measure of acclimation to the alien tissue of the donor by the recipient through the conditioning if not actual microchimerization associated with a gradual blending of the blood of the recipient with that of the donor. That is, rather than allowing a stark sudden confrontation and acute recoiling to the alien tissue, the period of blood reciprocal cross-circulation is extended and the relative proportion of donor blood slowly increased. In this way, a measure of tolerance induction is incorporated into the process of transplantation.

A sudden switched transplant is generally controlled manually by the operator or an assistant. In contrast, a metered switch is administered by a processing unit on the basis of sensor inputs and its prescription-program, the diversion chutes gradually moved from fully closed, that is, fully retracted, so that the circulatory systems of donor and recipient are completely isolated from one another, to fully open so that recipient circulation passes entirely through the donor heart. If left open long enough, the blood of donor and recipient-carefully matched before proceeding-would eventually become a 50/50 blend. If sudden-switched, the blood continuing to flow through the donor heart without interruption is that of the recipient. If meter-switched, the blood is that of the recipient blended with that of the donor.

Once the valves are completely open, that is, fully extended, so that the native heart of the recipient has been completely bypassed, the heart of the donor is clamped off and removed with the blood conveying lines, or mainlines, attached to it, and the recipient heart clamped off and removed, and the donor heart positioned in the chest of the recipient. To allow the postoperative use of the druglines (accessory channels) to directly pipe-target medication into the valves or bloodlines, these are allowed to remain in place. This is likewise the case when confluence conduits as described below in section 2b(1)(b)(b14), entitled Confluence Conduits: Connection of One or More Bloodlines to Flow through a Common Channel to the Same or a Different Number of Bloodlines with Self-leveling Apportionment of Outflow, are used.

While the bloodlines can serve as prosthetic and anastomosis avoided, this is generally reserved for a double heart transplant as addressed below in the section to follow. Ordinarily, as shown in FIGS. 22B thru 22E, 32D, and 32F, the stumps of the donor heart vessels are included in the graft, anastomosis accomplished, and the retained valves with accessory channels used as anastomosis bypasses to support the anastomoses with medication automatically released by the implanted prosthetic disorder response system on the basis of sensor inputs as directed by its prescription-program.

A critical advantage for the suppression of postoperative complications is not only the direct targeting of medication to the site without the need for reentry, but the ability of the control microprocessor to use the servovalves to bypass an anastomosis should it become adversely affected. Such an alternative route can be used indefinitely. Anastomosis is especially preferable in an infant or young child. Automatic ‘bailout’ thus is likewise administered by the fully implanted prosthetic disorder response system.

b. Double Heart Transplants. The Unsalvageable Native Heart Retains an Inadequate Ejection Fraction, or Ventricular Absolute Stroke Volume, and a Good Replacement Heart is not Available.

Type 2. Heterotopic heart transplant: When a good replacement heart is not available, this procedure, corresponding to a more conventional heterotopic, or ‘piggyback,’ heart transplant, is performed to supplement a marginal native heart with a second, stronger if less than ideal heart. The second heart is essentially used as an organic ventricular assist device that preserves pulsatile flow essential for normal development in a pediatric patient and is readily available in populous third world countries where ventricular assist devices are unaffordable.

The procedure is same as the Type 1 orthotopic transplant, except the intravascular diversion servovalves are stopped at about the half-way point depending upon the relative size of the added to the native heart, and although a position in the chest to the right side of the native heart is preferred, where surgical trauma, for example, makes positioning thus impossible, much consideration is given to the alternate site to be used. Heterotopic double heart metered compound vascular bypass, or switched-organ transplantation is addressed in the section that follows.

Type 3. Orthotopic then heterotopic heart transplant. When a good heart is not available, replace an irreparably defective native heart with two functional if less than ideal hearts.
3a. In the initial step, the native heart is replaced with the more robust of two replacement hearts in an ordinary metered compound vascular bypass, or switched-organ transplant.
3b. In the following step, a second replacement heart is added to the denervated first replacement heart in a procedure no different than the initial replacement of the native heart by metered compound vascular bypass. That is, the first replacement heart is treated as if it were that native. The substantial reduction in surgical trauma allows this second step to follow the first in a single procedure to further reduce trauma by eliminating the need for later reentry, which most often would be into the chest, leaving a second painful entry wound. Exceptionally, because a direct aortocaval connection in the lower abdomen does not require reentry into the chest, a double replacement operation can be accomplished in two separate steps, affording the patient an interval for recovery.

### Compound Vascular Bypass Transplantation of Humanized Porcine Hearts

Currently xenotransplantation of a humanized porcine heart is reserved for patients so ill that to provide them with a sound human heart would almost certainly risk its loss. Moreover, these patients are more susceptible to the serious side effects of mechanical assist devices to include “brain damage and infection” (Wolf, E., Reichart. B., Moretti, A., and Laugwitz, K. L. 2023. “Designer Pigs for Xenogeneic Heart Transplantation and Beyond, Online, Disease Models and Mechanisms (Cambridge, England) 16(5):dmm050177.

Given their precarious condition and the considerable trauma of a conventional heart transplant, the fundamental reduction in trauma and the elimination of the extensive dissection, cardioplegia, and cardiopulmonary bypass for the recipient and the avoidance of cold storage and ischemia-reperfusion injury for the graft organ involved in conventional heart transplant should materially improve the odds for a better outcome. Essentially, using the compound vascular bypass method leaves only the unavoidable midsternal thoracotomy or clamshell entry wound, for example, as the source of severe injury and lasting pain.

That whether for an allograft or a xenograft, a compound vascular bypass heart transplant does not involve the physical joining by direct anastomosis of the donor and recipient hearts means that at least anatomically and aside from physiology, the hearts need not match in size or in shape is another significant advantage. Yet another fundamental advantage is the monitoring of the implanted disorder response system for signs of rejection or infection to which the system is able to respond by pipe-targeting counteractants directly to the affected site or sites as well as systemically. The combination of a surgical method that eliminates much of the trauma and organ ischemia-reperfusion injury and the implanted automatic disorder response system to guard against hyperacute rejection should materially facilitate progress in this field.

The adverse surgical, life support, and anesthetic factors involved in a conventional heart xenograft are the same as in a conventional heart allotransplant, and with recipients necessarily impaired severely, certainly detract from the potential success of xenotransplantation as the one last chance for survival despite the chronically insufficient pool of sound human hearts. Another method for increasing the pool of available hearts is delineated just below in section 2b(1)(b)(b5), entitled Heterotopic Double Heart Metered Compound Vascular Bypass, or Switched-organ Transplantation [and so on].

Except that the donor need not have died while on life support, sustained in as life like a condition as possible, and humane treatment demands that the donor be anesthetized, the procedure for a compound vascular bypass of a humanized porcine heart would be much the same as that for an allograft where to allow for compresence of donor and recipient, access to a donor can be made proximate enough to avoid the need for perfusion system transport with its inherent deterrents as addressed above in section 2a(3, entitled Remotely Procured Graft Organ Preservation.

Albeit slowly, the transplantation of humanized xenogeneic graft organs, primarily porcine, is making headway (see, for example, Chrysakis, N., Magouliotis, D. E., Spiliopoulos, K., Athanasiou, T., Briasoulis, A., and 3 others 2024. “Heart Transplantation,” Online, MDPI [Multidisciplinary Digital Publishing Institute] Journal of Clinical Medicine (Basel, Switzerland) 13(2):558; Cooper, D. K. C. and Cozzi, E. 2024. “Clinical Pig Heart Xenotransplantation—Where Do We Go from Here?,” Online, Transplant International (Lausanne, Switzerland) 37:12592; Guo, Z. Lv. L., Liu, D., Ma. H., Wang. L., Fu. B., and Wang, F. 2024. “Network Meta-analysis: Effect of Cold Stress on the Gene Expression of Swine Adipocytes ATGL, CIDE4, UCP 2, and UCP 3,” Online, Current Issues in Molecular Biology (Basel, Switzerland) 46(5):3866-3876; Hussain, S. T., Kumar, A., Chan, J., James, L., Smith, D., and Moazami, N. 2024. “Anatomical Considerations and Surgical Technique of Porcine Cardiac Xenotransplantation,” Online, Journal of Thoracic and Cardiovascular Surgery Techniques (New York, New York) 25: 104-110; Jou, S., Mendez, S. R., Feinman, J., Mitrani, L. R., Fuster. V., and 3 others 2024. “Heart Transplantation: Advances in Expanding the Donor Pool and Xenotransplantation,” Nature Reviews. Cardiology (London, England) 21(1):25-36; Kozlov, M. 2024. “Pig-organ Transplants: What Human Recipients Have Taught Scientists,” Nature (Basingstoke, Hampshire, England) 629(8014):980-981; Lee, C.-H., Lee, H. J., Park, S.-W., Shin, J., Kang. S.-J. and 3 others 2024. “Mutational Analysis of Pig Tissue Factor Pathway Inhibitor a to Increase Anti-coagulation Activity in Pig-to-Human Xenotransplantation,” Biotechnology Letters (Dordrecht, South Holland, Netherlands) 46(4):521-530; Peterson, L., Yacoub, M. H., Ayares, D., Yamada, K., Eisenson, D. L, and 6 others 2024. “Physiological Basis for Xenotransplantation from Genetically Modified Pigs to Humans,” Online, Physiological Reviews (Bethesda, Maryland) 104(3):1409-1459; Raza, S. S. Hara, H., Eyestone, W., Ayares, D., Cleveland, D. C., and Cooper, D. K. C. 2024. “Pigs in Transplantation Research and Their Potential as Sources of Organs in Clinical Xenotransplantation,” Comparative Medicine (Memphis, Tennessee) 74(2):33-48; Schmauch, E., Piening, B., Mohebnasab. M., Xia, B., Zhu, C., and 51 others 2024. “Integrative Multi-omics Profiling in Human Decedents Receiving Pig Heart Xenografts,” Nature Medicine (New York, New York) 30(5):1448-1460; Schmoeckel, M., Langin, M., Reichart. B., Abicht, J. M., Bender, M., and 9 others 2024. “Current Status of Cardiac Xenotransplantation,” Online, Thoracic and Cardiovascular Surgeon (Stuttgart, Germany) 72(4):273-284; Sun, Q., Song. S. Y., Ma. J., Li, D., Wang. Y., Yang, Z., and Wang, Y. 2024. “Cutting Edge of Genetically Modified Pigs Targeting Complement Activation for Xenotransplantation,” Online, Frontiers in Immunology (Lausanne, Switzerland) 15:1383936; Vadori. M. and Cozzi. E. 2024. “Current Challenges in Xenotransplantation,” Online, Current Opinion in Organ Transplantation (Hagerstown, Maryland) 29(3):205-211; Yuan, Y., Cui. Y., Zhao, D., Yuan, Y., Zhao, Y., and 3 others 2024. “Complement Networks in Gene-edited Pig Xenotransplantation: Enhancing Transplant Success and Addressing Organ Shortage,” Online, [BioMed Central] Journal of Translational Medicine (London, England) 22(1):324; Zhou, Y., Zhou, S., Wang, Q., and Zhang, B. 2024. “Mitigating Cross-species Viral Infections in Xenotransplantation: Progress, Strategies, and Clinical Outlook,” Online, Cell Transplantation (Thousand Oaks, California) 33:9636897241226849; Anand, R. P., Layer, J. V. Heja. D., Hirose, T., Lassiter, G., and 56 others 2023. “Design and Testing of a Humanized Porcine Donor for Xenotransplantation,” Online, Nature (Basingstoke, Hampshire, England) 622(7982):393-401; Cooper, D. K. C. 2022. “The Long and Winding Road to Clinical Xenotransplantation: A Personal Journey,” Online, European Surgical Research (Basel, Switzerland) 63(4):165-172; Cooper, D. K. C. and Pierson, R. N. 3rd. 2023. “Milestones on the Path to Clinical Pig Organ Xenotransplantation,” Online, American Journal of Transplantation (New York, New York) 23(3):326-335; Denner, J. 2023. “Systemic Inflammation in Xenograft Recipients (SIXR) or Undetected PCMV/PRV [porcine cytomegalovirus/porcine roseolovirus] Transmission?,” Online, Xenotransplantation (Copenhagen, Denmark) 30(1):e12788; Ezzelarab, M. B., Hara, H., Ayares. D., and Cooper, D. K. C. 2023. “Systemic Inflammation in the Xenotransplant Recipient (SIXR) Can Occur in the Absence of pCMV Infection,” Online, Xenotransplantation (Copenhagen, Denmark) 30(2):e12796; Goerlich, C. E., Griffith, B., Hanna, P., Hong, S. N., Ayares, D., Singh, A. K., and Mohiuddin, M. M. 2023. “The Growth of Xenotransplanted Hearts Can Be Reduced with Growth Hormone Receptor Knockout Pig Donors,” Online, Journal of Thoracic and Cardiovascular Surgery (St. Louis, Missouri) 165(2):e69-e81; Habibabady, Z., McGrath, G., Kinoshita, K., Maenaka, A., Ikechukwu, I., and 6 others 2023. “Antibody-mediated Rejection in Xenotransplantation: Can It Be Prevented or Reversed?,” Online, Xenotransplantation (Copenhagen, Denmark) 30(4):e12816; Harris, A. G., Iacobazzi, D., Caputo, M., and Bartoli-Leonard, F. 2023. “Graft Rejection in Paediatric Congenital Heart Disease,” Online, Translational Pediatrics (Hong Kong, China) 12(8):1572-1591; Hoang. T. X. and Kim, J. Y. 2023. “Regulatory Macrophages in Solid Organ Xenotransplantation,” Online, Korean Journal of Transplantation (Seoul, Korea) 37(4):229-240; Koh, J., Chee, H. K., Kim, K. H., Jeong. I. S., Kim, J. S., Lee, C. H., and Seo, J. W. 2023. “Historical Review and Future of Cardiac Xenotransplantation, Online, Korean Circulation Journal (Seoul, Korea) 53(6):351-366; Konstantinov I E, Cooper D K C, Adachi I, Bacha E, Bleiweis M S, and 13 others 2023. “Consensus Statement Heart Xenotransplantation in Children: Toward Clinical Translation,” Journal of Thoracic and Cardiovascular Surgery (St. Louis, Missouri) 166(3):960-967; Moazami, N., Stern, J. M., Khalil, K., Kim, J. I., Narula, N., and 38 others 2023. “Pig-to-Human Heart Xenotransplantation in Two Recently Deceased Human Recipients,” Nature Medicine 29(8):1989-1997; Mohiuddin, M. M., Singh, A. K., Scobie, L., Goerlich, C. E, Grazioli, A., and 22 others 2023. “Graft Dysfunction in Compassionate Use of Genetically Engineered Pig-to-Human Cardiac Xenotransplantation: A Case Report,” Lancet (London, England) 402(10399):397- 410; Montgomery, R. A. and Tang. W. H. W. 2023. “Cardiac Xenotransplantation: A New Frontier for Advanced Heart Failure,” Online, Current Treatment Options in Cardiovascular Medicine (New York, New York) 25(3):65-78; Reichart, B., Cooper, D. K. C., Langin, M., Tonjes, R. R., Pierson, R. N., and Wolf, E. 2023. “Cardiac Xenotransplantation: From Concept to Clinic,” Online, Cardiovascular Research (London, England) 118(18):3499-3516; Schizas, N., Nazou, G., Angouras, D. C., Iliopoulos, D. C. Dedeilias, P., and Argiriou M. 2023. Heart Xenotransplantation: Current Issues and Perspectives,” Online, Journal of Tehran Heart Center (Tehran, Iran) 18(2):79-81; Boulet, J., Cunningham, J. W., and Mehra. M. R. 2022. “Cardiac Xenotransplantation: Challenges. Evolution, and Advances,” Online, Journal of the American College of Cardiology. Basic to Translational Science (New York, New York) 7(7):716-729; Chaban, R., Cooper, D. K. C., and Pierson, R. N. 3rd. 2022. “Pig Heart and Lung Xenotransplantation: Present Status,” Online, Journal of Heart and Lung Transplantation (New York, New York) 41(8):1014-1022; Eisenson, D. L., Hisadome. Y., and Yamada, K. 2022. “Progress in Xenotransplantation: Immunologic Barriers, Advances in Gene Editing, and Successful Tolerance Induction Strategies in Pig-to-Primate Transplantation, Online, Frontiers in Immunology (Lausanne, Switzerland) 13:899657; Goerlich, C. E. Singh, A. K., Griffith, B. P., and Mohiuddin, M M. 2022. “The Immunobiology and Clinical Use of Genetically Engineered Porcine Hearts for Cardiac Xenotransplantation,” Online, Nature Cardiovascular Research (London, England) 1(8):715-726; Griffith, B. P., Goerlich, C. E., Singh, A. K., Rothblatt, M., Lau, C. L., and 8 others 2022. “Genetically Modified Porcine-to-Human Cardiac Xenotransplantation, Online, New England Journal of Medicine (Boston, Massachusetts) 387(1):35-44; Lei, T. Chen, L., Wang, K., Du. S., Gonelle-Gispert, C., Wang. Y., and Buhler, L. H. 2022. “Genetic Engineering of Pigs for Xenotransplantation to Overcome Immune Rejection and Physiological Incompatibilities: The First Clinical Steps,” Online, Frontiers in Immunology (Lausanne, Switzerland) 13:1031185; Raza, S. S., Hara, H., Cleveland, D. C., and Cooper, D. K. C. 2022. “The Potential of Genetically Engineered Pig Heart Transplantation in Infants with Complex Congenital Heart Disease,” Online, Pediatric Transplantation (Copenhagen, Denmark) 26(5):e14260; Wang, W., He, W., Ruan, Y., and Geng, Q. 2022. “First Pig-to-Human Heart Transplantation,” Online, Innovation (Cambridge, Massachusetts) 3(2):100223; Zhou, Q., Li, T., Wang, K., Zhang, Q., Geng, Z., and 3 others 2022. “Current Status of Xenotransplantation Research and the Strategies for Preventing Xenograft Rejection,” Online, Frontiers in Immunology (Lausanne, Switzerland) 13:928173; Hara, H., Iwase. H., Nguyen, H., Miyagawa, Y., Kuravi, K., and 5 others 2021. “Stable Expression of the Human Thrombomodulin Transgene in Pig Endothelial Cells is Associated with a Reduction in the Inflammatory Response,” Online, Cytokine (Oxford, England) 148:155580; Patel, P. M., Connolly, M. R., Coe. T. M., Calhoun, A., Pollok, F., and 5 others 2021. “Minimizing Ischemia Reperfusion Injury in Xenotransplantation, Online, Frontiers in Immunology (Luasanne, Switzerland) 12:681504; Reichart. B., Langin, M., Denner, J., Schwinzer, R., Cowan, P. J., and Wolf. E. 2021. “Pathways to Clinical Cardiac Xenotransplantation,” Online, Transplantation (Hagerstown, Maryland) 105(9):1930-1943; Pierson, R. N. 3rd, Burdorf, L., Madsen, J. C., Lewis, G. D., and D′Alessandro, D. A. 2020. “Pig-to-Human Heart Transplantation: Who Goes First?,” Online, American Journal of Transplantation (New York, New York) 20(10): 2669-2674; Yamamoto, T., Cui. Y., Patel, D., Jagdale, A., Iwase, H., Ayares, D., Cooper, D. K. C., and Hara, H. 2020. “Effect of Intravenous Immunoglobulin (IVIg) on Primate Complement-dependent Cytotoxicity of Genetically Engineered Pig Cells: Relevance to Clinical Xenotransplantation,” Online, [Nature] Scientific Reports (London, England) 10(1):11747; Cleveland, D., Banks, C. A., Hara. H., Carlo, W. F., Mauchley, D. C., and Cooper, D. K. C 2019. “The Case for Cardiac Xenotransplantation in Neonates: Is Now the Time to Reconsider Xenotransplantation for Hypoplastic Left Heart Syndrome?,” Pediatric Cardiology (New York, New York) 40(2):437-444; Iwase. H., Liu, H., Li, T., Zhang, Z., Gao, B., and 7 others 2017. “Therapeutic Regulation of Systemic Inflammation in Xenograft Recipients,” Online, Xenotransplantation (Copenhagen, Denmark) 24(2):10.1111/xen.12296; Ezzelarab. M. B., Ekser, B., Azimzadeh, A., Lin, C. C., Zhao. Y., and 9 others 2015. “Systemic Inflammation in Xenograft Recipients Precedes Activation of Coagulation,” Xenotransplantation (Copenhagen, Denmark) 22(1):32-47; Bulato, C., Radu, C., and Simioni, P. 2012. “Studies on Coagulation Incompatibilities for Xenotransplantation,” Methods in Molecular Biology (Totowa, New Jersey) 2012:885:71-89).

Additional references pertaining to pig-to-baboon intraabdominal and otherwise conventional heterotopic transplantation are provided next below at the end of section 2b(1)(b)(b5), entitled Heterotopic Double Heart Metered Compound Vascular Bypass, or Switched-organ Transplantation [and so on].

2b(1)(b)(b5). Heterotopic double heart metered compound vascular bypass, or switched-organ transplantation, and the use of unified entry arterial and venous confluence conduits to avoid disuse atrophy when the graft organ is positioned in the lower abdomen whether by direct aortocaval connection.

In contrast to a conventional heterotopic transplant where the allogeneic added ventricles are sutured to the native atria, in a compound vascular bypass heterotopic transplant, the hearts remain physically separate, without incision, and distinct as to allow substantially separate diagnosis and treatment. While improbable, the elimination of trauma and ischemia from the original operation plus the continuous monitoring and support of the implanted disorder response system medicinally and with respect to synchronization make it more likely for either heart to recover. Then, at least in a potential sense, the other heart would become recoverable for reimplantation in another recipient.

Although supported by the added ventricles, the trauma and ischemia of the original conventional heterotopic heart transplant and the lack of constant monitoring and support militate against the recovery of the recipient heart, making its recovery to competent function next to impossible, precluding recovery of the added ventricles for reimplantation in another recipient. Accordingly, the insults imposed on the hearts during the original procedure as well as that which would be necessary to separate the two make the prospect of recovery so improbable as to contemplate such an eventuality as a later reversal without justification.

Confluence conduits are addressed below in section 2b(1)(b)(b14), entitled Confluence Conduits: Connection of One or More Bloodlines to Flow through a Common Channel to the Same or a Different Number of Bloodlines with Self-leveling Apportionment of Outflow. Metered compound vascular bypass, or switched-organ transplantation in an adult with the use of unified entry arterial and venous exit confluence conduits provides 1. Self-leveling among outlet bloodlines when different in number than those inlet. 2. Allows common medication of the confluent flow, 3. Makes possible the incorporation of an inline axial flow pump in the conduit lumen to pump the flow forward or backward, and 4. Clarifies the surgical field by reducing the number of bloodlines needed to connect corresponding vessels of the donor and recipient.

As described below in this section, when the added donor heart in a double heart heterotopic compound vascular bypass transplant is positioned in the lower abdomen with direct aortocaval connection, the connections afforded by a confluence conduit avoid the disuse atrophy which had long been observed in solid organ transplants prepared for immunological studies.

Confronted with a patient suffering from end stage heart failure, the practitioner has four alternatives: a conventional orthotopic, heart transplant, a conventional heterotopic, heart transplant, the placement of a biventricular assist device, or a total artificial heart. In that the native ventricles are replaced with mechanical rather than allograft ventricles, a total artificial heart is essentially similar to a conventional heart transplant.

Both have drawbacks, but the mechanical device has more, such as the need for an extracorporeal source of power, the fact that it is not consistent with the function of living tissue, weighs three times as much as an equivalent heart, is subject to wear out, and may generate heat. Detractions pertinent to the conventional as compared to a metered compound vascular bypass heterotopic heart transplant are addressed above in section 2a, entitled Background of the Invention. General. Placed in a child with an unsurvivably malformed heart, a mechanical circulatory assist device will not grow, making certain the need for further painful operations.

Here it is asserted that compared to all of the foregoing, a metered compound vascular bypass orthotopic and probably a heterotopic heart transplant are fundamentally superior. This is based upon the complete elimination of surgical trauma and ischemia to both the graft and innate hearts, as well as the elimination of extended cardiopulmonary bypass and general anesthesia. The gradual cross-microchimerization inherent in the reciprocal cross-circulation used to seamlessly transfer the graft organ from the circulatory system of the donor into that of the recipient incorporates tolerance induction as intrinsic in the process.

Moreover, follow-up monitoring and immediate therapeutic response by the fully implanted automatic disorder response system that administered the transplant alleviates almost all of the anxiety experienced by a patient who has cheated death. Devised to evade the major causes for complications and poor outcomes encountered with conventional procedures and apparatus, some problems that will eventuate with the proposed procedure will undoubtedly eventuate over time; however, except for infection and inflammation which are universal, those problems are unlikely to duplicate the serious problems encountered with any conventional procedure or apparatus.

End stage heart failure exigent, a sound replacement heart will often be unavailable in time to save the patient, making the need for a heart transplant in less time critical, this especially in third world countries where mechanical circulatory devices are not likely to be available. However, a metered compound vascular bypass heterotopic, that is, the addition of aa second, heart transplant can be the best of these choices even in the developed world. If a sound replacement heart is available, then the trauma of resecting, that is, excising, the native ventricles of both the recipient heart and the donor hearts to replace these with the ventricles of the donor heart in a conventional heart transplant, or with an artificial heart, or the surgery to implant a biventricular assist device, ischemia-reperfusion injury, and extended operational duration while under cardiopulmonary bypass life support would cooperate to materially reduce the likelihood of a favorable outcome.

There is growing evidence that the surgical and ischemic trauma incurred before and during a conventional heart transplant is a significant factor in the cardiac allograft vasculopathy if not cancer likely to later necessitate another traumatizing heart transplant. A means for seamlessly transferring the heart from the circulatory system of a donor into the circulatory system of a recipient without these insults allows a more encouraging outcome with a less than perfect replacement or second heart, and reduce the standard for the condition of a replacement heart from sound to less than perfect but functional thus increasing the pool of available hearts. In contrast, if performed in accordance with the metered compound bypass method, orthotopic replacement of the defective with a sound heart or the heterotopic addition of a second heart to assist that native in generating an adequate ejection fraction inflicts zero trauma on either the native or the added heart.

The trauma which cannot be eliminated is due to factors unrelated to the donor-to-recipient transfer procedure but rather the unavoidable need to enter through a potentially long-term painful access incision as well as the side effects of the drugs used which would pertain in any operation. By comparison, once healed, the preparation of a space to house the second heart is not likely to result in continued pain. The metered compound vascular heterotopic double heart method is distinct from and superior to a conventional heterotopic heart transplant whereby the donor and native hearts are merged into a single organ rather than left separate to allow their separate automatic monitoring and treatment by the disorder response system.

The individualized directly pipe-targeted delivery of medication to either heart allows optimal treatment, and should the native heart recover to provide an adequate ejection fraction, the potential removal of the heart that was added contributes an additional heart to the pool. The direct conjunction of the two hearts in a conventional heart transplant necessitates a substantial sameness in size, and sutures along the very lines of greatest mechanical and immunological stress in the circulatory system. The semi-independence of native and graft hearts means not only that either can be treated apart from the other but that the two need not be reasonably matched in size, which factor increases the pool of available hearts. In most cases, placement of the added heart in the chest is preferred, and the fact that the added heart can be smaller reduces the dissection needed to make room for it in the chest as preferred.

As indicated, if later removed as no longer needed, the allograft can be considered for placement in another recipient. In a metered double heart compound vascular bypass operation, the only factors that impose surgical trauma are those ineradicable-incision into the chest, and the dissection needed to create a space to place the second heart. However, factors other than the surgical procedure and anesthesia as such are also ineradicable with any heart transplant-impairment of the immune system and the risk of kidney damage. But with a conventional heart transplant, these combine with the surgical trauma to jeopardize the outcome, still leaving the compound bypass method preferable.

Artificial hearts developed to date have significant shortcomings. “The life of a total artificial heart is about 5 years,” (https://www.bannerhealth.com/services/transplant/heart-transplants/artificial-heart- transplant), and “Less than 60% of people with a total artificial heart survive for one year after surgery,” (https://www.nhlbi.nih.gov/health/total-artificial-heart/what-to-expect #). Current immunosuppressants not only render the patient susceptible to infection, but “Immunosuppressants, which you will have to take for the rest of your life, can cause major kidney damage,” (https://www.medicinenet.com//how_long_you can_you_live_after_a_heart_transplant/article.htm). Yet another adverse factor is that a patient with end stage heart failure will as a result have multiple organ damage. But the significance of the trauma these results assume cannot be withdrawn from the end data.

In most of the world, a biventricular assist device is unavailable and unaffordable, but more importantly from a medical standpoint, these impart continuous rather than pulsatile blood flow, which is associated with a higher rate of postoperative stroke than is a total artificial heart, which does provide pulsatile flow. Similarly, Fontan circulation following a three-stage repair of a severely congenitally malformed heart in a neonate, also continuous rather than pulsatile, is also known to lead to numerous deficiencies in development. However, to provide pulsatile flow with a “total” artificial heart requires a high capacity power source with a need for frequent recharging. In contrast, an organic heart imposes no need for an external source of power.

Too large to implant, an artificial heart makes it necessary for the patient to wear a power pack—a major impediment and detraction from the quality of life (see, for example, Arabia, F. A. and Murray, C. F. 2023. “The Total Artificial Heart: Where Have We Been, Where are We Now, Where are We Going?,” Indian Journal of Thoracic and Cardiovascular Surgery (New Delhi, India) 2023 39 (Supplement 1):198-205; Henn, M. C. and Mokadam, N. A. 2022. “Total Artificial Heart as a Bridge to Transplantation,” Current Opinion in Organ Transplantation (Hagerstown, Maryland) 27(3):222-228; Chung, J. S., Emerson, D., Megna, D., and Arabia, F. A. 2020. “Total Artificial Heart: Surgical Technique in the Patient with Normal Cardiac Anatomy,” Online, Annals of Cardiothoracic Surgery (Hong Kong, China)(2):81-88; Melton, N., Soleimani, B., and Dowling, R. 2019. “Current Role of the Total Artificial Heart in the Management of Advanced Heart Failure,” Current Cardiology Reports (Philadelphia, Pennsylvania) 21(11):142; Cheng, A., Trivedi, J. R., Van Berkel, V. H., Massey, H. T., and Slaughter, M. S. 2016. “Comparison of Total Artificial Heart and Biventricular Assist Device Support as Bridge-to-transplantation,” Journal of Cardiac Surgery (Hoboken, New Jersey) 31(10):648-653. Cook, J. A., Shah, K. B., Quader, M. A., Cooke, R. H., Kasirajan, V., and 4 others 2015. “The Total Artificial Heart,” Online, Journal of Thoracic Disease (Hong Kong, China) 7(12):2172-2180).

Were the process described above in section 2b(1)(b) and depicted in FIGS. 15 and 16 for orthotopic heart transplantation using the metered compound vascular bypass, or switched-organ method stopped at the half-way point depending upon the relative sizes and absolute stroke volumes, or ejection fractions of the donor and recipient hearts after the donor heart had been removed and its vascular stumps replaced with an artificial heart of anastomosed and sustained on extracorporeal membrane oxygenation, then each heart would be left to contribute the sum heart blood volume and drive half the normal volume of blood in the double heart circulatory system.

Addressed below in section 2b(1)(b)(b14), entitled Confluence Conduits: Connection of One or More Bloodlines to Flow through a Common Channel to the Same or a Different Number of Bloodlines with Self-leveling Apportionment of Outflow, the use of confluence conduits can considerably reduce the intricacies and if pertinent, the targeted differential treatment in common of the set of confluent vessels. This is uniquely valuable in dealing with the numerous vessels in a heart transplant.

If the azygos factor is any indication, here where the venous return to either heart is reduced to about half less the blood the added heart contributed—not just ten percent when both venae cavae are clamped—this condition of reduced volume should not result in injury to organs or tissues nor should it induce a dysrhythmia due to intracardiac hypovolemia, at least on a short-term basis. This should even out, and if a real factor, can be controlled with synchronization electronics. By virtue of continued perfusion, a solid organ transplant inherently perpetuates both microchimerization and the risk of rejection.

Given that donor and recipient hearts exchange equal volumes of blood and upon harvesting, the donor heart contributes its fill of blood to the recipient, a condition of hypovolemia in the former is unlikely even early in the process of transfer into the circulatory system of the recipient; toggling, addressed above in section 2a, entitled Background. General, in the subsection entitled Servovalve Toggling in Switched Organ Heterotopic Double Heart Transplantation, and resynchronization are mentioned in an abundance of caution as resources to counteract such eventualities. However improbable, to deal with such an eventuality, the concept of toggling is provided.

A central object in both orthotopic and heterotopic compound vascular bypass, or switched-organ transplantation undertaken to overcome end stage failure and/or pulmonary vascular resistance is that the graft organ be subjected to zero ischemia time, and that it be left intact, that is, not internally breached and reconstructed thereby inflicting much trauma upon and weakening it. Only ten of the numerous previous efforts to heterotopically transplant an accessory heart in the abdomen are referenced below in section 2b(1)(b)(b16), entitled Direct Aortocaval Connection: Unified Entry Arterial and Exit Venous Confluence Conduits for the Heterotopic Connection of a Graft Heart in the Lower Abdomen. None conform to these basic measures for considerably reducing trauma.

In addition to the recipient thoracotomy and laparotomy (celiotomy, abdominotomy) which already sapping, often result in weight loss and muscle wasting but are unavoidable, most conventional heart transplants add to this burden of trauma. Recovery following trauma whether due to surgery is often marked by some wasting (see, for example, Zhang, B. H., Ghazi Faisal, S., Ruo, L., Simunovic, M., Pinto-Sanchez, M. I., and Serrano, P. E. 2022. “Weight Loss following Hepatopancreatobiliary Surgery. How Much is Too Much? A Retrospective Cohort Study,” Online,” Surgical Innovation 29(2):195-202; Hirsch, K. R., Wolfe, R. R., and Ferrando, A. A. 2021. “Pre- and Post-surgical Nutrition for Preservation of Muscle Mass, Strength, and Functionality following Orthopedic Surgery,” Online, MDPI [Multidisciplinary Digital Publishing Institute] Nutrients (Basel, Switzerland) 13(5):1675; Cusack, B. and Buggy, D. J. 2020. “Anaesthesia, Analgesia, and the Surgical Stress Response,” Online, British Journal of Anaesthesia Education 20(9):321-328; Hew, J. J., Parungao, R. J., Shi, H., Tsai, K. H., Kim, S., and 5 others 2020. “Mouse Models in Burns Research: Characterisation of the Hypermetabolic Response to Burn Injury,” Burns 46(3):663-674; Finnerty, C. C., Mabvuure, N. T., Ali, A., Kozar, R. A., and Herdon, D. N. 2013. “The Surgically Induced Stress Response,” Online, Journal of Parenteral and Enteral Nutrition, 37(50):21S-29S).

In many cases, the graft organ will have undergone an ischemic time of over an hour, cold blood cardioplegia, storage of the graft organ in ice-cold saline, and excision of the graft interatrial septum and tricuspid valve. Both donor and recipient hearts are more or less bisected so that the resulting transplant actually comprises parts of the recipient as well as the donor heart in a complementary fitting together as a single heart.

Both hearts traumatized, these are now sutured together in immediate contact, so that the native portion having been made integral with the donor portion will be immediately affected by rejection or infection, for example, of the donor portion. Given a poor outcome, the parts respective of the donor and recipient cannot be separated; rather, such requires orthotopic replacement through retransplantation with a competent heart. By the same token, if the recipient heart had been in less than end stage failure, regained normal function to a degree as to recommend its reinstatement as a single unit, or the donor portion were to become adversely affected, to disengage the recipient portion from the combination is ill advised if even possible.

While the original heterotopic technique has been improved, much of these detractions remain valid, so that patients who could have recovered from heart failure should not have been irreversibly modified as to necessitate a need for immunosuppressants for life (see, for example, Vaijyanath, P. 2020. “Heterotopic Heart Transplant: Relevance as Bio-VAD in Emerging Economies,” Online, Indian Journal of Thoracic and Cardiovascular Surgery (New Delhi, India) 36 (Suppl 2):279-286; Novitzky, D., Cooper, D. K., and Barnard, C. N. 1983. “The Surgical Technique of Heterotopic Heart Transplantation,” Online, Annals of Thoracic Surgery (Amsterdam, North Holland) 36(4):476-482; Barnard, C. N. and Losman, J. G. 1975. “Left Ventricular Bypass,” South African Medical Journal (Cape Town, South Africa) 49(9):303-12).

Discomfort due to the enlarged size of the merged structure is likely temporary. Where a compound vascular heterotopic double heart transplant leaves the hearts untraumatized and distinct from one another, conventional heterotopic heart transplant adds competent ventricles to the recipient heart to create a compound unit. In both conventional orthotopic and heterotopic heart transplantation, the native and graft portions are inseparable for diagnosis or treatment. In contrast to this, should the native heart recover and the other symptoms of impaired function subside or be manageable with medication, and the patient is able to undergo the surgery, a compound vascular bypass transplanted second heart can be removed, eliminating a continued need for immunosuppressants.

Heart failure is no longer considered completely irreversible; when adverse symptoms other than the reduced ejection fraction persist, whether a second assistance heart can be explanted depends upon whether these symptoms are too severe for the patient to be considered a candidate for another open procedure or can be managed medically, something an implanted automatic disorder response system can provide apart from or with a heart transplant. Heart failure with preserved ejection fraction despite high ventricular filling pressure due to obesity and/or diabetes can be reversed through weight reduction to avoid the need for a heart transplant (Chockalingam 2022 cited below).

The need for a heart transplant is associated with heart failure with reduced ejection fraction. Accordingly, patients who had received a double heart much less an orthotopic compound vascular bypass heart transplant would be those whose heart failure had been without preserved ejection fraction and could at least to some extent, recover by losing weight and adopting a healthier lifestyle. However, comorbidity in heart failure can be sufficiently complex as to justify the implantation of a hierarchical control-based automatic disorder response system even when the need for a heart transplant is not indicated.

If not a complete cure, recovery from heart failure is possible (see also, for example, Salyer L G, Salhi H E, Brundage E A, Shettigar V. Sturgill S L, and 12 others 2024. “Troponin I Tyrosine Phosphorylation Beneficially Accelerates Diastolic Function,” Online, Circulation Research (Baltimore, Maryland) 134(1):33-45; Stoicescu, L., Crişan, D., Morgovan, C., Avram, L., and Ghibu, S. 2024. “Heart Failure with Preserved Ejection Fraction: The Pathophysiological Mechanisms behind the Clinical Phenotypes and the Therapeutic Approach,” Online, MDPI [Multidisciplinary Digital Publishing Institute] International Journal of Molecular Sciences (Basel, Switzerland) 25(2):794; Cameli, M. Birks, E. J., and Landra, F. 2023. “Editorial: Insights in Heart Failure and Transplantation: 2022,” Online, Frontiers in Cardiovascular Medicine (Lausanne, Switzerland) 10:1290011; Chrysakis, N., and Xanthopoulos. A., Magouliotis, D., Starling, R. C., Drakos, S. G., Triposkiadis, F., Skoularigis, J. 2023. “Myocardial Recovery,” Online, MDPI [Multidisciplinary Digital Publishing Institute] Diagnostics (Basel, Switzerland) 13(8):1504; Kasiakogias, A., Ragavan, A., and Halliday, B. P. 2023. “Your Heart Function Has Normalized—What Next after TRED-HF [treatment for recovered dilated heart failure (dilated cardiomyopathy)]?,” Online, Current Heart Failure Reports (Philadelphia, Pennsylvania) 20(6):542-554; Malik, A., Brito, D., Vagar, S., and Chhabra, L. 2023. “Congestive Heart Failure,” Online, Treasure Island, Florida: StatPearls Publishing Co.; McElderry, B., O'Neill. T., Griffin, B. P., Kalahasti, V., Barzilai, B., and Brateanu, 2023. “A. Factors Associated With Maintenance of an Improved Ejection Fraction: An Echocardiogram-based Registry Study,” Online, Journal of the American Heart Association (Oxford, England) 12(21):e031093; Prana Jagannatha, G. N., Suastika, L. O. S., Kosasih, A. M., de Liyis, B. G., Yusrika, M. U., and 8 others 2023. “Prognostic Value of Baseline Echocardiographic Parameters in Heart Failure with Improved vs Nonrecovered Ejection Fraction,” Online, Canadian Journal of Cardiology Open (New York, New York) 5(12):859-869; Sawalha, K., Asad, O., Tadisina, S., Alalawi, L., Mahmood, M., Alkhatib, D., and Alexander, T. 2023. “Obesity-induced Hyperglycemia and Heart Failure Preserved Ejection Fraction: Uncharted Territories to Remission, Online, Cureus (Palo Alto, California) 15(11):e49178; Cameli. M., Birks, E. J., and Landra, F. 2022. “Editorial: Highlights in Heart Failure and Transplantation in 2021,” Online, Frontiers in Cardiovascular Medicine (Lausanne, Switzerland) 9:877515; Chockalingam. A. 2022. “Obesity- Years” Burden May Predict Reversibility in Heart Failure with Preserved Ejection Fraction,” Online, Frontiers in Cardiovascular Medicine (Lausanne, Switzerland) 9:821829; Gevaert, A. B., Kataria, R., Zannad, F., Sauer, A. J., Damman, K., and 3 others 2022. “Heart Failure with Preserved Ejection Fraction: Recent Concepts in Diagnosis, Mechanisms, and Management,” Online, Heart (British Cardiac Society)(London, England) 108(17):1342-1350; Hajouli, S. and Ludhwani. D. 2022. “Heart Failure and Ejection Fraction,” Online, Treasure Island, Florida: StatPearls Publishing Co; Hullin, R., Meyer, P., Yerly, P., and Kirsch, M. 2022. “Cardiac Surgery in Advanced Heart Failure,” Online, MDPI [Multidisciplinary Digital Publishing Institute] Journal of Clinical Medicine (Basel, Switzerland) 11(3):773; Vejpongsa, P., Torre-Amione, G., Marcos-Abdala, H. G., Kumar, S., Youker, K., Bhimaraj. A., and Nagueh, S. F. 2022. “Long-term Development of Diastolic Dysfunction and Heart Failure with Preserved Left Ventricular Ejection Fraction in Heart Transplant Recipients,” Online, Scientific Reports 12(1):3834; Yoshihisa, A., Sato Y., Kanno, Y., Takiguchi, M., Yokokawa. T., and 8 others 2020. “Prognostic Impacts of Changes in Left Ventricular Ejection Fraction in Heart Failure Patients with Preserved Left Ventricular Ejection Fraction,” Online, Open Heart (London, England) 7(1):e001112; Harper, A. R., Patel, H. C., and Lyon, A. R. 2018. “Heart Failure with Preserved Ejection Fraction,” Online, Clinical Medicine (London, England) 18 (Supplement 2):s24-s29; Wolsk, E., Claggett. B., Kober, L., Pocock, S., Yusuf, S., and 5 others 2018. “Contribution of Cardiac and Extra-cardiac Disease Burden to Risk of Cardiovascular Outcomes Varies by Ejection Fraction in Heart Failure,” Online, European Journal of Heart Failure (Chichester, England) 20(3):504-510; Katsi, V., Georgiopoulos, G., Laina, A., Koutli, E., Parissis, J., and 3 others 2017. “Left Ventricular Ejection Fraction as Therapeutic Target: Is It the Ideal Marker?,” Heart Failure Reviews (Norwell, Massachusetts) 22(6):641-655; Nadruz, W. Jr., West. E., Santos, M., Skali, H., Groarke, J. D., Forman, D. E., and Shah, A. M. 2016. “Heart Failure and Midrange Ejection Fraction: Implications of Recovered Ejection Fraction for Exercise Tolerance and Outcomes,” Online, Circulation. Heart Failure (Hagerstown, Maryland) 9(4):e002826; Basuray, A., French, B., Ky, B., Vorovich, E., Olt, C., and 3 others 2015. “Response to Letter Regarding Article, “Heart Failure with Recovered Ejection Fraction: Clinical Description, Biomarkers, and Outcomes,” Online, Circulation (Hagerstown, Maryland) 131(6):e344; Psaty, B. M., Shah, S. J., and Gottdiener, J. 2015. “Letter by Psaty et al. Regarding Article. “Heart Failure with Recovered Ejection Fraction: Clinical Description, Biomarkers, and Outcomes,” Online, Circulation (Hagerstown, Maryland) 131(6):e343; Basuray, A., French, B., Ky, B., Vorovich, E., Olt. C., and 3 others 2014. “Heart Failure with Recovered Ejection Fraction: Clinical Description, Biomarkers, and Outcomes,” Online, Circulation (Hagerstown, Maryland) 129(23):2380-2387).

Due to the trauma and ischemia-free treatment of the graft organ during the compound vascular bypass double heart heterotopic transplant operation and the constant vigilance and support provided by the implanted disorder response system which had administered the transplantation as well as a prescribed regimen of moderate exercise and proper diet, the odds for the native heart of the recipient to recover will have been considerably improved over the foregoing conventionally favorable outcomes. Hence, as a realistic prospect, a method for transferring the added heart to another patient while preserving the fundamental benefits of trauma and ischemia-free transplantation is warranted.

Following a compound vascular bypass heterotopic double heart transplant, removal and seamless transfer of the allograft heart to a second recipient is by transferring flow through the allograft from the native heart of the initial recipient, which can be sudden, to that of the successor, which is best gradual, or metered to effectuate an initial measure of microchimerization in order to reduce the intensity of an otherwise stark immune confrontation. During a metered transplant, in addition to those entrusted to the control system, the administration of immune tolerance inducing agents periprocedurally on an as-needed basis is understood.

Seamless and Ischemia-Free Explantation and Transfer of an Allograft Second Heart from a First to a Second Recipient

While placement of the second booster or assist heart in the chest is preferred, sections 2b(1)(b)(b16), entitled Direct Aortocaval Connection: Unified Entry Arterial and Exit Venous Confluence Conduits for the Heterotopic Connection of a Graft Heart in the Lower Abdomen; 2b(1)(b)(b17), entitled Doubly Heterotopic Double Heart Compound Vascular Bypass, or Switched-organ Transplantation; and 2b(1)(b)(b18), entitled Hybrid Doubly Heterotopic Double Heart Transplantation below describe alternative positions depending upon the anatomical and medical context of the patient. Matching and microchimerization in this operation is among three, not just two individuals. The graft organ crossmatched to each of the recipients, two things sufficiently compatible with a third would reasonably be sufficiently compatible with one another as well.

For positioning in the lower abdomen, a flexible confluence conduit such one made with the highly flexible as well as expandable structure described in section 2b(2)(f), entitled Expandable Structure to Accommodate Growth and Self-adjust in Size in Vascular Segment Prostheses, Lines, and Confluence Conduits Placed in Children, below but reinforced to traverse the waist without kinking when the patient bends over can be provided. However, to overcome gravity, the heterotopic double heart should be strong enough to drive the blood up to the chest, preferably without the need for the support of a positive inotrope.

If such robust graft is not available, the next best heart that is must be assisted by an inline axial pump such as indicated in the drawing figures as part number 167 positioned in the conduit passing up to the native heart in the chest. Due to the energy demand involved, this should remain only until a suitable second heart becomes available. The process for replacement of the temporary graft organ with the second is the same as that delineated herein for switching an allograft heart from a first to a second recipient. In these switches of a donor heart from one recipient to another, the arrangement shown in FIG. 15B applies, the distinctions pertaining only to the length and type of bloodlines or conduits based upon the positioning of the second heart.

In FIG. 15B, reciprocal cross circulation between the allograft and the native heart of the first recipient is through the loop to the right controlled by the vascular servovalves in the upper and lower right-hand corners. Upon connection of the native heart of the second recipient is connected, flow is gradually transferred from the right to the left-hand loop under the control of all four servovalves. Figures such as 15B are not to be understood as including all of the corresponding vessels of the subjects but rather to the connections between a subset thereof.

If only a single vessel is involved the conduit is a bloodline 196. If plural vessels are included, the conduit is a confluence conduit such as the upper conduit in FIG. 15C. In a heart transplant, the arterial vessels will be plural while those venous will be bloodlines conveying the flow of the venae cavae. The representation in FIG. 15C as showing the conduits with flats and vaults denotes not a distinction between a bloodline and a confluence conduit but rather that either is extendable for use in a small child.

Unlike a conventional heterotopic heart transplant wherein only the ventricles of the donor are anastomosed to the openings left after resecting away the ventricles of the native heart of the recipient, in a compound vascular bypass heart transplant, the added heart is implanted intact. This fundamentally liberalizes the operation in allowing the separate positioning, separate directly pipe-targeted medicinal and electrostimulatory therapy, and even a significant difference in the size of the allograft compared to the native organ. Such differences will mean an extension in durability from ten or so years to a far longer period.

By the same token, for positioning in the chest to the right of the native heart, this allows the use of a smaller robust graft, whereas for positioning in the lower abdomen, a larger heart with a stronger ejection fraction able to perfuse the native heart and the lungs in the chest would be adequate. In an exigent circumstance, if a more robust heart is unavailable, an inline axial pump can be used to assist the flow of blood craniad against the force of gravity. While possible as an extrapolation based upon the examples provided herein, at least in one operation using two surgical teams, the implantation of both a second and a third less than perfect heart is not contemplated as necessitating an unacceptable extent of dissection and susceptibility to complications in a patient who is by definition already debilitated.

Placement in the lower abdomen is with 1: The protection of a surrounding cage as described in section 2b(1)(b)(b6) following, entitled Heart Cage for the Infrarenal Abdominal Positioning of a Second Heart, 2. A flexible and for use in a child, an expandable conduit described below in section 2b(2)(f), entitled Expandable Structure to Accommodate Growth and Self-adjust in Size in Vascular Segment Prostheses, Lines, and Confluence Conduits Placed in Children, and if necessary, an inline axial booster pump such as indicated by part number 167 in the drawing figures.

Discouraged is the use of a confluence conduit or expansion structured tube with an inline axial pump such as represented by part number 167 in the drawing figures. As opposed to a real heart, a mechanical assist device imposes an onerous impediment in the need for an exogenous source of energy, both transcutaneous energy transfer or the need to wear an energy pack exacting material impairment in the quality of life.

Regardless of the original position of the implant, reentry should avoid retracing the original entry wound incision (see, for example, Valente, T., Bocchini, G., Rossi, G., Sica, G., Davison, H., and Scaglione, M. 2019. “MDCT [multidetector computed tomography] Prior to Median Re-stemnotomy in Cardiovascular Surgery: Our Experiences, Infrequent Findings, and the Crucial Role of Radiological Report,” Online, British Journal of Radiology (London, England) 92(1101):20170980; Imran Hamid, U., Digney, R., Soo, L., Leung, S., and Graham, A. N. 2015. “Incidence and Outcome of Re-entry Injury in Redo Cardiac Surgery: Benefits of Preoperative Planning,” Online, European Journal of Cardiothoracic Surgery (Oxford, England) 47(5):819-823). Among the complications encountered upon reentry are adhesions, addressed above in section 2a. Background of the Invention. General, to include their prevention and detection during formation.

The means and method for transferring perfusion of the donor organ from the first to the second recipient are addressed above section 2b, entitled Vascular Valves and Servovalves in the Diversion of Blood Flow, in the subsection thereof entitled One-, Two-, and Three-way passive side-entry Flow Diversion Jackets, the two-way passive flow diversion jacket shown in FIG. 10G used for the purpose. Control of the transfer between circulatory systems is by the arterial servovalves in the upper corners of FIG. 15B and the venous servovalves in the lower corners, the three-way side-entry arterial and venous side-entry Flow Diversion Jackets serving to define the three subcircuits involved but otherwise passive during this process.

FIG. 15B provides a schematized depiction of the circuit used during the transfer of an allograft organ here a heart, from the circulatory system of a first to a second recipient. Switching of the vascular servovalves at the four corners of the figure is by the master control microprocessor is always simultaneous and antipodally complementary, that is, the two-way arterial flow diversion jacket at the top center, and the venous flow diversion jacket at the bottom center passive, the valves on the right initially provide the entire flow to the graft and then gradually reduce and finally shut off the flow towards the graft, while the valves on the left have synchronously opened to accept the full flow to support the graft.

As in any organ transplant, recipients and donors are matched prior to commencing the procedure and tolerance inducing methods are applied pre- and perioperatively. Passive one-, two-, and three-way side-entry flow diversion jackets can be made with passages large enough to avoid damage to red blood cells as occurs in the use of a cardiopulmonary bypass machine which can result in damage to the kidneys and intestines. As shown in FIGS. 10F thru 10H, all passive side-entry flow diversion jackets are provided with at least one drugline to allow the direct pipe-targeting into it of an essential drug such as a heparin or steroid.

By eliminating the need for cardiopulmonary bypass and blood transfusion during surgery, the compound vascular bypass method eliminates its serious complications regardless of the specific causes (see, for example, Bhirowo, Y. P., Raksawardana, Y. K., Setianto, B. Y., Sudadi, S., Tandean, T. N., and 4 others 2023. “Hemolysis and Cardiopulmonary Bypass: Meta-analysis and Systematic Review of Contributing Factors,” Online, BioMed Central Journal of Cardiothoracic Surgery (London, England) 18(1):291; Vercaemst, L. 2008. “Hemolysis in Cardiac Surgery Patients Undergoing Cardiopulmonary Bypass: A Review in Search of a Treatment Algorithm,” Online, Journal of ExtraCorporeal Technology (Les Ulis Cedex, ile-de-France, France) 40(4):257-267).

Since factors related to hemolysis during cardiopulmonary bypass may be unrelated to direct physical injury to red blood cells, and factors in addition to cardiopulmonary bypass are involved, the ascription to hemolysis by life support apparatus of kidney injury, which often follows a conventional heart transplant, may at least in part, be misattributed (see, for example, Gale, D., Al-Soufi, S., MacDonald, P., and Nair, P. 2024. “Severe Acute Kidney Injury Postheart Transplantation: Analysis of Risk Factors,” Online, Transplantation Direct (Philadelphia, Pennsylvania) 10(3):e1585; Han, L., L,i L., Linghu, H., Zheng, L., and Gou, D. 2023. “Cardiopulmonary Bypass in a Rat Model May Shorten the Lifespan of Stored Red Blood Cells by Activating Caspase-3,” Online, PLoS [Public Library of Science] One (San Franisco, California) 18(9):e0290295; Kant, S., Banerjee, D., Sabe, S. A., Sellke, F., and Feng, J. 2023. “Microvascular Dysfunction following Cardiopulmonary Bypass Plays a Central Role in Postoperative Organ Dysfunction,” Online, Frontiers in Medicine (Lausanne, Switzerland) 10:1110532; Jocher, B. M., Schilling, J. D., Fischer, I., Nakajima, T., Wan, F., and 5 others 2021. “Acute Kidney Injury Post-heart Transplant: An Analysis of Peri-operative Risk Factors,” Online, Clinical Transplantation (Copenhagen, Denmark) 35(6):e14296; Leballo, G., Moutlana, H. J., Muteba, M. K., and Chakane, P. M. 2021. “Factors Associated with Acute Kidney Injury and Mortality during Cardiac Surgery,” Online, Cardiovascular Journal of Africa (Durbanville, South Africa) 32(6):308-313; Zakkar, M., Guida, G., Suleiman, M. S., and Angelini, G. D. 2015. “Cardiopulmonary Bypass and Oxidative Stress,” Online, Oxidative Medicine and Cellular Longevity (New York, New York) 2015:189863; Salaria, O. N., Barodka, V. M., Hogue, C. W., Berkowitz, D. E., Ness, P. M., Wasey, J. O., Frank, S. M. 2014. “Impaired Red Blood Cell Deformability after Transfusion of Stored Allogeneic Blood but Not Autologous Salvaged Blood in Cardiac Surgery Patients,” Online, Anesthesia and Analgesia (Baltimore, Maryland) 118(6):1179-1187; Vermeulen Windsant, I. C., de Wit, N. C., Sertorio, J. T., van Bijnen, A. A., Ganushchak, Y. M., and 5 others 2014. “Hemolysis during Cardiac Surgery is Associated with Increased Intravascular Nitric Oxide Consumption and Perioperative Kidney and Intestinal Tissue Damage,” Online, Frontiers in Physiology (Lausanne, Switzerland) 5:340; Morariu, A. M., Gu, Y. J., Huet, R. C., Siemons, W. A., Rakhorst, G., and Oeveren, W. V. 2004. “Red Blood Cell Aggregation during Cardiopulmonary Bypass: A Pathogenic Cofactor in Endothelial Cell Activation?,” Online, European Journal of Cardiothoracic Surgery (Oxford, England) 26(5):939-946).

As indicated, the two-way flow diversion jackets shown in FIGS. 10G and 15B maintain two subcircuits which are kept separate until merged in confluent craniad flow at the center. Entry into the arterial and venous circuits of the first recipient is controlled by the servovalves in the corners to the right, while entry into the arterial and venous circuits of the second recipient is controlled by the servovalves in the corners to the left, the shift in flow gradually transferred synchronously from the first to the second recipient. As in FIG. 15A, the flow of blood through the arterial and venous vessels in FIG. 15B have been summarized into a single vessel which the use of confluence conduits would approximate.

At the start of the process, the valves on the right, depending upon the relative strength, that is, ejection fraction, of the two hearts, are extended to approximately the half-way position where these valves had been set as long as the second heart had remained implanted in the first recipient. Accordingly, the flow of blood through the native heart is evenly shared with the graft heart to the exclusion of the rest of the circuit and the second recipient.

Also at the outset, the servovalves on the left are fully retracted so that blood flow is kept separate and apart from the joint (common, wider) circuit. As the operation proceeds, the valves on the right gradually extend to draw, or tap, off a small volume of flow for entry into the common circuit. The gradual retraction of the valves of the right to tap or draw off a lesser volume of blood is synchronous with the gradual extension of the valves on the left to pass through a greater volume of blood.

This process gradually closes off the valves on the right from the common circuit to shift the equal sharing of blood flow with the donor heart with the native heart of the second recipient. At the completion of the action, the diversion chutes 18 of the valves on the right have been fully retracted constraining all flow through the portion of the common circuit of the first recipient to the right whose circulatory system is restored to its original independence of an accessory heart. That is, the diversion chutes of the valves on the right, now fully retracted constrain the entire volume of flow to the native heart of the first recipient, perfusion of the graft heart having been transferred from the native heart of the initial to the second recipient.

In the improbable eventuality that the donor heart were to improve while that native of the first recipient continued to degrade as to risk an irreversible arrhythmia and sudden cardiac arrest, the system would fully retract the diversion chutes to close off flow to the native heart leaving that added to support the recipient. Following the recovery procedure, whether to remove the native heart and resituate that added in the mediastinum would depend upon whether the patient had experienced discomfort.

In some experiments, despite genetic modification of the donor to reduce the extreme disparity between and better align the genetics of the different species used, adding xenotransplantation into the mix only complicated finding a solution that would advance medical practice by helping patients with congenital heart defects or end-stage heart failure. In a compound vascular bypass heterotopic or double heart transplant, when the graft organ is removed from the donor, it is cut off from its circulatory system, likely resulting in a brief period of hypovolemia after it has been placed in the recipient.

The plasticity of the neonatal circulatory system is addressed above in section 2a(1), entitled Congenital Defects of the Heart and Other Organs—The Importance of Prompt Intervention. This is, however, tempered by the fact that the graft brings with it about 280 milliliters, or 9.5 ounces, of blood, and just as the loss of blood following a blood donation, the hematopoietic system, immediately prompted by the loss in pressure, begins to replenish the shortage. Moreover, unless the donor had been on life support long enough to damage the red blood cells, additional blood can be transfused, while at the same time accomplishing an additional if negligible measure of microchimerization.

Otherwise, the deficit is readily remedied by means of an ordinary blood transfusion and special measures in the delivery room to reverse respiratory complications (see, for example, Sankaran, D., Lane, E. C. A., Valdez, R., Lesneski, A. L. and Lakshminrusimha, S. 2022. “Role of Volume Replacement during Neonatal Resuscitation in the Delivery Room,” Online, MDPI [Multidisciplinary Digital Publishing Institute] Children (Basel, Switzerland) 9(10):1484; Sankaran, D., Vali, P., Chandrasekharan, P., Chen, P., Gugino, S. F., and 15 others 2021. “Effect of a Larger Flush Volume on Bioavailability and Efficacy of Umbilical Venous Epinephrine during Neonatal Resuscitation in Ovine Asphyxial Arrest,” Online, MDPI [Multidisciplinary Digital Publishing Institute] Children (Basel, Switzerland) 8(6):464). In exceptional cases, toggling, a method for alternating the volume of blood delivered into the native and accessory hearts, can be incorporated into the implanted prosthetic disorder response system master control microprocessor prescription-program.

A compound bypass, or switched-organ, double heart transplant most often comprises the addition of a second heart to one significantly impaired by means of a switched-organ heart transplant as delineated in the preceding section where the second heart remains in place. Otherwise, placement of a second heart can be positioned as the condition of the patient will allow, to include in the lower abdomen. This can be by doubly heterotopic compound vascular metered heart transplantation whereby the added heart is positioned in the lower abdomen without local connection, or through direct aortocaval connection as delineated below in section 2b(1)(b)(b16), entitled Direct Aortocaval Connection: Unified Entry Arterial and Exit Venous Confluence Conduits for the Heterotopic Connection of a Graft Heart in the Lower Abdomen, rather than by a vessel-to-vessel connection between native and donor organs.

Confluence conduits and unified confluence conduits are described below in section 2b(1)(b)(b14), entitled Confluence Conduits: Connection of One or More Bloodlines to Flow through a Common Channel to the Same or a Different Number of Bloodlines with Self-leveling Apportionment of Outflow. When a severely implored native heart is to be replaced by two functional but less than perfect hearts, the procedure is accomplished in two steps whereby the native heart is replaced by an orthotopic metered switched-organ transplant and the second heart is added through a heterotopic metered switched-organ transplant.

To implant two hearts with no more than one in the chest is probably feasible but discouraged as inviging needless complications such as an insufficiency of blood pressure requiring the use of inline pump equipped confluence conduits of which the energy requirement would limit it to temporary use. Larger in luminal diameter than individual bloodlines, an inline pump, part number 167 in the drawing figures is readily accommodated in confluence conduit where it can propel to increase or retard to lower the blood pressure. While placement of the accessory heart in the abdomen using the heterotopic double heart metered compound vascular bypass, or switched-organ transplant scheme does not involve direct aortocaval connection, the distance from the chest to the abdomen is still be

For greater dependability in an adult and to accommodate growth and avoid the need for revision in a child, the confluence conduit is made of the fabric described below in section 2b(2)(f), entitled Expandable Structure to Accommodate Growth and Self-adjust in Size in Vascular Segment Prostheses, Lines, and Confluence Conduits Placed in Children. Placement thus without complete protection by the ribs requires the use of a protective metal gridwork as described in the following section, 2b(1)(b)(b6), entitled Heart Cage for the Infrarenal Abdominal Positioning of a Second Heart.

A temporary and nugatory condition of hypovolemia following harvesting of the donor heart can be prevented by leaving the venous, or donor/graft second heart return valves open briefly just prior to harvesting, or it can be left for the hematopoietic system of the recipient, possibly microchimerized, to bring the sum volume of blood up to the normative value. A need for transfusion should never arise. The pain of thoracic entry wounds notwithstanding, switched-organ transplantation should often eliminate the need for general anesthesia, and therewith, its complications. Assiduously matched and tolerance induced immunologically before, during, and after transplantation, the arousal of an alarming transfusion reaction should prove rare or nonexistent.

As ensues spontaneously following blood donation or subcritical hemorrhage allowed to resolve spontaneously, the renin-angiotensin-aldosterone hormone system restores to and maintains the sum volume of blood at the normotensive level (see, for example, Avital, G., Snider, E. J., Berard, D., Vega, S. K., Hernandez, Torres, S. I., and 3 others 2022. “Closed-loop Controlled Fluid Administration Systems: A Comprehensive Scoping Review,” Online, Journal of Personalized Medicine 12(7):1188; Fountain, J. H. and Lappin, S. L. 2022. “Physiology, Renin Angiotensin System,” Online, Treasure Island, Florida: StatPearls Publishing Co.; Sharma, R. and Sharma, S. 2022. “Physiology, Blood Volume,” Online, Treasure Island, Florida: StatPearls Publishing Co.; Suddock, J. T. and Crookston, K. P. 2022. “Transfusion Reactions,” Online, Treasure Island, Florida: StatPearls Publishing Co.; Ohara, S., Nishimura, A., Tachikawa, A. and Iijima, T. 2020. Effect of Remifentanil on Intraoperative Fluid Balance: A Retrospective Statistical Examination of Factors Contributing to Fluid Balance,” Online, Dental Anesthesia and Pain Medicine 20(3):129-135; Aubron, C., Aries, P., Le Niger, C., Sparrow, R. L., and Ozier, Y. 2018. “How Clinicians Can Minimize Transfusion-related Adverse Events?,” (English and French), Transfusion Clinique et Biologique 25(4):257-261; Munoz-Durango, N., Fuentes, C. A., Castillo, A. E., Gonzalez-Gomez, L. M., Vecchiloa, A. Fardella, C. E., and Kalergis, A. M. 2016. “Role of the Renin-Angiotensin- Aldosterone System beyond Blood Pressure Regulation: Molecular and Cellular Mechanisms Involved in End-organ Damage during Arterial Hypertension,” Online, MDPI [Multidisciplinary Digital Publishing Institute] International Journal of Molecular Sciences (Basel, Switzerland) 17(7):797; Joosten, A., Alexander, B., Delaporte A. Lilot, M., Rinehart, J., and Cannesson, M. 2015. “Perioperative Goal Directed Therapy Using Automated Closed-loop Fluid Management: The Future?,” Online, Anesthesiology Intensive Therapy (Lublin, Poland) 47(5):517-523; Iijima, T., Brandstrup, B., Rodhe, P., Andrijauskas, A., and Svensen, C. H. 2013. “The Maintenance and Monitoring of Perioperative Blood Volume,” Online, BioMed Central. Perioperative Medicine (London, England) 2(1):9).

In this way, a switch type heterotopic heart transplant, which leaves both donor and recipient hearts intact, not only eliminates the trauma of a conventional heterotopic heart transplant but accomplishes a measure of immune tolerance-inducing microchimerism intrinsic in the reciprocal cross-transfusion used to transfer the organ directly from the circulatory system of the donor into the circulatory system of the recipient. Essentially, the trauma is limited to the need to gain access through an entry wound in the chest, which absent adequate anesthesia, is notoriously painful. The placement of a second heart to support that native both ameliorates the shortage of graft organs and is a relatively inexpensive alternative to the use of mechanical assist devices or artificial hearts in third world countries where a single mechanical device can be reserved for short term use in successive patients as a bridge to a double heart solution.

During evolution of the higher mammals (see, for example, Genain, M. A., Morlet, A., Herrtage, M. Muresian M., Anselme, F., and 4 others 2018. “Comparative Anatomy and Angiography of the Cardiac Coronary Venous System in Four Species: Human, Ovine, Porcine, and Canine,” Journal of Veterinary Cardiology 20(1):33-44; Picciucchi, S., Barone, D., Sanna, S., Dubini, A., Goodman, L. R., and 6 others 2014. “The Azygos Vein Pathway: An Overview from Anatomical Variations to Pathological Changes,” Insights into Imaging 5(5):619-628), were either the superior or the inferior vena cava to have been congenitally anomalous (see, for example, Shakerian, B., Razavi, N., and Mandegar, M. H. 2021. “Inferior Vena Cava Agenesis in an Adult Organs Donor,” International Journal of Organ Transplantation Medicine 12(1):52-53; Spentzouris, G., Zandia, A. Cesmebasi, A., Kinsella, C. R., Muhleeman, M. and 5 others 2014. “The Clinical Anatomy of the Inferior Vena Cava: A Review of Common Congenital Anomalies and Consideration for Clinicians,” Clinical Anatomy (New York, New York) 27(8):1234-1243; Luo, X.-L., Qian, G.-N., Xiao, H., Zhao, C.-L., and Zhou, X.-D. 2012. “Posterior Nutcracker Syndrome Associated with Left Inferior Vena Cava with Azygos Continuation and Retroaortic Right Renal Vein,” Korean Journal of Radiology 13(3):345-348; Malaki, M., Willis, A. P., and Jones, R. G. 2012. “Congenital Anomalies of the Inferior Vena Cava,” Clinical Radiology 67(2):165-171; Bass, J. E., Redwine, M. D., Kramer, L. A., Huynh, P. T., and Harris, J. H. Jr. 2000. “Spectrum of Congenital Anomalies of the Inferior Vena Cava: Cross-sectional Imaging Findings,” Radiographies 20(3):639-652) or to have become obstructed, individuals peculiar in having an alternative passage back into the superior vena cava would nevertheless survive.

The azygos vein would add about ten percent to the volume of the venous return arriving through the intact vena cava, so that something greater than half the normal venous return would persist with adequate volume to sustain life. In this, the azygos was likely selected for and fixed by heredity as collateral circulation that allowed individuals whose vena cava had become at least partially obstructed to survive-essentially, the same type of collateral circulation that arises to circumvent a coronary artery which has become obstructed by plaque, for example.

It was this finding, the ‘azygos factor’ and ‘azygos flow principle,’ as an alternative path for the return of blood should the venae cavae become obstructed, originally reported in 1952 by Andreasen and Watson in the British Journal of Surgery (Andreasen, A. T. and Watson, F. 1952. “Experimental Cardiovascular Surgery,” British Journal of Surgery (Oxford, England) 39(158):548-551), that C. Walton Lillehei and his coworkers depended upon to initiate the era of open heart surgery.

This suggests that the provocation of a dysrhythmia can be avoided by frequently diverting more than half the normal volume of blood to either of the two hearts, and by providing the implanted master control microprocessor with dysrhythmia sensors and resynchronization circuitry. The previous sections on metered switch organ transplantation have been based upon established precedent as reported in the literature, with the result that these are substantially free of conjecture. For compound vascular bypass solid organ transplantation, much less double heart transplantation, no precedent and no literature exists.

Many experiments have involved the implantation into a muridae, leporidae (lagomorpha), suidae, or canidae of a second heart, but never with the arterial and venous interconnections between the two hearts delineated above, never with the operation performed as described above, or with the immediate support of an automatic response system able to adjust the venous retum volume, target drugs or electrostimulation to, and if necessary, resynchronize either or both hearts. This lack of experience and the large number of physiological and therapeutic variables involved preclude addressing the subject with much confidence. In this circumstance, the complications more likely to be encountered, the measures needed to counteract and overcome these, and the technical means to effectuate these measures are best set forth, hesitancy notwithstanding.

Arranging for two impaired hearts to each drive half the normal volume of blood compared to either driving a full complement of blood alone might be conceived of as making little if any difference. However, this is not a proper characterization of the action of each heart or of the two working together. The diversion chutes have setpoints at the halfway point of each, adjusted for the relative ejection fraction of either heart, but are controlled to move to either aorta and main pulmonary artery, or pulmonary trunk, just short enough of the peaks in systolic volume to maintain sufficient carryover venous return volume and continuity of function in the other heart.

The action can be viewed as the alternate discharge of either heart in close enough succession for the volume and pressure of the successive ejections from either heart to present an additive effect sufficient to duplicate those of a normal ejection fraction. Adjustment in the interval separating the systoles passed into the aorta from either heart allows adjustment in the combined ejection fraction, and the rate of adjustment can be used to encourage reverse remodeling and recovery, as well as to avert hypertension and remodeling, that is, degenerative change in the heart overall.

Alternation between the hearts at the rate set by the implanted control microcontroller or microprocessor means that when the diversion chutes are positioned to favor either heart, that heart is ejecting most of the blood it would alone but at a lower frequency. Such represents a reduction in load through time, and since this frequency is controllable, the opportunity for reverse remodeling and restoration without the risk of atrophy or a loss in contractile function were either heart subject to disuse (Brinks, H., Tevaearai, H., Muhlfeld, C., Bertschi. D., Gahl, B., Carrel. T., and Giraud, M. N. 2009. “Contractile Function is Preserved in Unloaded Hearts Despite Atrophic Remodeling,” Journal of Thoracic and Cardiovascular Surgery 137(3):742-746).

As the frequency of switching between the hearts is a determinant in the optimization of battery power, to preserve a major advantage of double heart transplantation over a ventricular assist device in being fully implanted rather than requiring the wearing of paracorporeal apparatus, selection of the second heart should consider the number of systoles between the hearts that should occur to effectively reconstitute a normal ejection fraction. The action can be visualized as analogous to that of a side-by-side inline twin combustion engine in which the stroke of either piston alternates, where a second heart added rather than both transplanted, the one is given and the other selected to achieve the combined power output sought.

Two hearts driving half the normal volume of blood would essentially drive 2×½ or the same volume of blood as either alone, and shifting the setpoints of the servovalves for a difference in size or ejection fraction would only result in a relatively larger proportion of the unit performance contributed by the more adequate of the two. If equal hearts beat in synchrony with half the volume of blood or out of synchrony while the valves remain adjusted to half open makes little difference. However, if the valves are left fully open and are beating in synchrony, the first heart will eject most if not all of the load, which is little different than were it solitary, with any blood passed to the second heart adding a negligible fraction if any to ejection.

If however, the hearts with valves open beat asynchronously, depending upon the heartrates, the second heart will receive a larger volume of blood, often, a full load. Toggling then, seeks to queue the driving of successive full loads by either heart. If this is excessive as might result in pulmonary hypertension, for example, the valves can be adjusted to produce the desired joint ejection fraction. The intentional imposition of asynchrony by implanting an artificial pacemaker to optimize the additive effect of each heart driving the full volume of blood warrants study alone as well as in combination with toggling. In some cases, this might allow the servovalves to remain idle with their accessory channels used to pipeline target drugs to either heart as the implanted sensors indicate the need therefor. However, having mediated the transplantation, the servovalves are already in position, so that to remove or replace these in a follow-up endoscopic procedure would pose a gratuitous risk and imposition on the patient, often elderly and frail.

Significantly, since if allowed to beat spontaneously, that is, left free-running with all valves ordinarily retracted, or fully open, the two hearts would seldom beat in synchrony anyway, so that a need for intervention if any would increase in probability the more poorly matched were the hearts to produce a normal ejection fraction together. That is, the better matched on the basis of competency is the second or both transplant hearts at the outset, the less needed will be such alternate switching or “toggling,” and the expenditure of energy required to sustain such function. By the same token, the presence of such function liberalizes the usability of hearts over a range of sizes and levels of competency that would otherwise have to be rejected at the relatively small expense of more frequent replacement or recharging of a battery in a surface port or transcutaneous recharging of the battery in a fully implanted system.

In an infant or toddler, the imposition upon freedom of movement due to a need to wear paracorporeal apparatus likely retards, and hindering if not blocking continuity of development over a developmental window, will likely interfere with if not preclude, normal motor development. This consequence is especially onerous, since the impediment to free movement is not neurobiological but mechanical and avoidable (see, for example, Flensborg-Madsen, T., Gronkjær, M., and Mortensen, E. L. 2019. “Predictors of Early Life Milestones: Results from the Copenhagen Perinatal Cohort,” Online, BioMed Central Pediatrics 19(1):420; Neelon, B., Shoaibi. A., and Benjamin-Neelon, S. E. 2019. “A Multivariate Discrete Failure Time Model for the Analysis of Infant Motor Development,” Statistics in Medicine 38(9):1543-1557; Flensborg-Madsen, T., Gronkjær, M., and Mortensen, E. L. 2019. “Predictors of Early Life Milestones: Results from the Copenhagen Perinatal Cohort,” Online, BioMed Central Pediatrics 19(1):420; Gajewska, E., Sobieska, M., Kaczmarek, E., Suwalska, A., and Steinbom, B. 2013. “Achieving Motor Development Milestones at the Age of Three Months May Determine, but does Not Guarantee, Proper Further Development,” Scientific World Journal 2013:354218).

In contrast to a conventional heterotopic transplant, in a switch-organ heart transplant, the hearts are connected in series-parallel, the diversion servovalves positively directing the blood through all the vessels of the accessory as well as the native heart, dispelling disuse atrophy as a cause for potential graft failure. Moreover, the blood is immediately controllable as to the relative apportionment of blood as between either heart, and such correction can be applied automatically on the basis of sensor data feedback provided to the implanted control microprocessor.

Thus, in a switch heterotopic transplant, it is only when the hearts-which in a conventional heterotopic as in a switched-organ double heart heterotopic transplant, beat independently (see, for example, Chu, S. H., Wang, S. S., Tsai, C. H., Wu, C. C., Lee, C. M., and 4 others 1995. “Monitoring of Two Hearts in One Chest,” Journal of Cardiovascular Surgery (Turin, Italy) 36(5):449-454; Yusuf. S., Mitchell, A., Yacoub, M. H. 1985. “Interrelation between Donor and Recipient Heart Rates during Exercise after Heterotopic Cardiac Transplantation,” British Heart Journal 54(2):173-178; (see also for example, Cowell, et al., Frank et al., and Morris-Thurgood et al., references below)—beat in synchrony, that their combined ejection fraction is least, so that were the two to have the same ejection fraction, their combined ejection fraction would equal roughly the full (reduced) ejection fraction of either. Viewed thus, pacing, if used at all, would be necessary to assure separate timing between the two if the hearts beat simultaneously, which except intermittently, is improbable over any significant interval.

This is because when the hearts beat simultaneously, there is no opportunity for the diversion chutes to be switched from the one to the other in order to divert most of every few successive systoles of each heart. The switching need not—or from the standpoint of conserving battery power-should not catch every systole of either heart, but visit each heart with sufficient frequency to approximate a normal ejection fraction. Followed by the same consequence in the opposite heart, the net or overall ejection fraction for both should fall within the normal range. Given that in metered switch double heart transplantation the connections between the hearts cause the blood to flow through both hearts with equal volume over the course of a few beats, it is doubtful that evidence from conventional heterotopic heart transplantation, where the hearts are not so interconnected but rather semi-independent, and best synchronized with the aid of a pacemaker, applies.

The consequences of beat independence in conventional heterotopic transplantation, with respect to a need for synchronization, where one of the pair of added ventricles might malfunction with no internal means for effecting correct proportioning, relative disuse atrophy, retention of contractile function, reverse remodeling, and other aspects of the conventional procedure are addressed in the literature (Kem, M. J., Lim, M. J., and Goldstein, J. A. 2018. Hemodynamic Rounds: Interpretation of Cardiac Pathophysiology from Pressure Waveform Analysis, Hoboken, New Jersey: John Wiley and Sons; Laredo, M., Varnous, S., Balagny, P., Leprince, P., Hidden-Lucet, F., and Duthoit, G. 2017. “Successful Catheter Ablation of Recipient Left Ventricular Tachycardia 26 Years after Heterotopic Heart Transplantation,” HeartRhythm Case Reports 3(3):159-163; Laredo, M., Varnous, S., Balagny, P., Leprince, P., Hidden-Lucet, F., and Duthoit, G. 2017. “Reply to the Editor-Rhythmologic Challenges following Heart Transplantation,” [refers to Laredo, M., Vamnous, S., Balagny, P., Leprince, P., Hidden-Lucet, F., and Duthoit, G. 2017, Op cit., just preceding] HeartRhythm Case Reports 3(12):608-609; Sindermann, J. R., Welp. H. A., Pott, C., and Monnig, G. 2017. “To the Editor-Rhythmologic Challenges following Heart Transplantation,” HeartRhythm Case Reports 3(12):608; Engelen, M. A., Stypmann, J., Bittner, A., Scheld, H. H., Sindermann, J. R., Breithardt, G., and Eckardt, L. 2011. “Complex Ventricular Arrhythmias in Patients after Heterotopic Heart Transplantation,” International Journal of Cardiology 152(1):e9-e11; Fiorelli, A. I., Coelho, G. H., Lima, J. L., Lourenço, D. D., Gutierres, P., and 4 others 2009. “Massive Degeneration and Atrophy of the Native Heart after Heterotopic Transplantation: A Case Report,” Transplantation Proceedings 41(3):965-966; Frank, C. M., Razavi, M., Saeed, M., Bogaev, R. C., and Massumi. A. 2009. “Recognition and Management of Complex Rhythm Disorders in Heterotopic Heart Transplantation,” Journal of Heart and Lung Transplantation (New York, New York) 28(3):294-296; Lai. H. Y., Chen, J. H., Chiu, K. M., Wang, K. L., Cheung, W. K., Li, A. H., and Chu, S. H. 2008. “C T of Two Hearts Beating in One Chest,” American Journal of Roentgenology 191(6):1711-1716; Smith, R. D. Jr., Radovancevic, B., Gregoric. I. D., Cohn, W. E., and Frazier, O. H. 2006. “Orthotopic Retransplantation in Heterotopic Transplant Recipients: 3 Case Reports,” Journal of Heart and Lung Transplantation (New York, New York) 25(2):245-247; Aris. A. 1999. “Images in Cardiothoracic Surgery. Biphasic Pulse Wave Due to Asynchronous Heart Contractions in Heterotopic Cardiac Transplantation,” Annals of Thoracic Surgery 68(5):1866; Beyer, E., Vatcharasiritham, C., Sweeney, M., Przybylowski, P., Delgado, R. 3rd, Radovancevic, B., and Frazier, O. H. 1998. “Linked Pacing after Heterotopic Heart Transplantation with Concurrent Left Ventricular Reduction of the Native Heart,” Texas Heart Institute Journal 25(4):299-302; Wang. S. S., Chu, S. H., Ko. W. J., Chen, Y. S., and Chou, N. K. 1998. “Heterotopic Heart Transplantation for Severe Pulmonary Hypertension,” Transplantation Proceedings 30(7):3408-3409; Gatewood, L. B., Larson, D. F., Bowers, M. C., Bond, S., Cardy, A., Sethi, G. K., and Copeland, J. G. 1996. “A Novel Mechanism for Cyclosporine: Inhibition of Myocardial Ischemia and Reperfusion Injury in a Heterotopic Rabbit Heart Transplant Model,” Journal of Heart and Lung Transplantation (New York, New York) 15(9):936-947; Cowell. R. P., Morris-Thurgood, J., Coghlan, J. G., Ilsley, C. D., Mitchell, A. G., and 3 others 1995. “Effects of Paced Counterpulsation on Exercise Capacity and Hemodynamics after Heterotopic Heart Transplantation,” American Journal of Cardiology 75(5):415-417; Cowell, R., Morris-Thurgood, J., Coghlan, J., Paul. V., Mitchell, A., and 3 others 1994. “Postoperative Hemodynamic Improvement with Paced Linkage of the Donor and Recipient Hearts following Heterotopic Cardiac Transplantation,” Clinical Cardiology 17(10):542-546; Morris-Thurgood, J., Cowell, R., Paul. V., Kalsi, K., Seymour, A. M., and 4 others 1994. “Hemodynamic and Metabolic Effects of Paced Linkage following Heterotopic Cardiac Transplantation,” Circulation 90(5):2342-2347; Rigaud, M., Bourdarias, J. P., e1 Khoury, E., Beauchet. A., Labedan, F., and 3 others 1992. “Hemodynamic Evaluation of Heterotopic Heart Transplantation,” Journal of Thoracic and Cardiovascular Surgery 104(2):248-255; Kern, M. J., Deligonul. U., and Miller, L. 1991. “Interpretation of Cardiac Pathophysiology from Pressure Waveform Analysis: Extra Hearts: Part I.,” Catheterization and Cardiovascular Diagnosis 22(3):197-204; Dor. V., Jourdan, J., Bourlon, F., Elbeze, J. P., Grinneiser, D., Isetta, C., and Montiglio, F. 1986. “Performance of Recipient Hearts after Heterotopic Transplantation,” Thoracic and Cardiovascular Surgeon (Stuttgart, Germany) 34(5):295-299; additional references cited above in sections 2a and 2b(1)(b)(b3), entitled Neonatal and Infant Compound Vascular Bypass, or Switched-organ, Transplantation of the Heart, below in this section, and incidentally in other sections). Furthermore, while pacemakers are used, following a conventional heterotopic transplant, neither is there anything like the implanted automatic disorder response system to continue postprocedural support by directly targeting drugs to either or both hearts.

Seen in this light, to attempt to synchronize the hearts, through the use of local electrical or optogenetic stimulator probes or a cardioverter defibrillator emitting sub-shock energy level discharges, would be precisely counterproductive. This factor results in a considerable reduction in power consumption, making it possible to use transcutaneous recharging of an implanted battery, and eliminate the impedimenta such as the need to wear a power pack with ventricular assistance devices. To achieve a sufficient combined ejection fraction, the added heart must be more competent the less competent is that native, which is a central consideration is second heart acceptance.

As in conventional heterotopic heart transplant, the hearts beat separately, that is, absent control by an artificial cardiac pacemaker, are not locked in phase; however, the load on both is reduced, allowing recuperation with reverse remodeling, and by switching a sufficient proportion of the systoles of either heart in alternation by diversion chute toggling, ejection by either should should alternate frequently enough in the unified outflow stream to effectively increase the net ejection fraction, or absolute stroke volume.

A sufficient load is maintained on both hearts to minimize the susceptibility of either to fibrillation, but were either heart to defibrillate, almost all flow would be directed through the unaffected heart, and this apportionment continued until help was reached. In this connection, it is important to understand that the servovalves at the venous entry portals, that is, the superior and inferior venae cavae, can be adjusted from passing all blood through the first, usually the native heart in the succession, thus bypassing the added heart, or the reverse, that is, completely bypassing the first heart by directing all blood into the added heart.

There is, then, no need for an additional valve to switch between the two. To pass all blood through the first heart, no blood is diverted from that heart into the second; the servovalves are completely opened, that is, the diversion chutes are completely retracted, while to bypass the first heart, the valves are completely closed, that is, fully extended. This full range is, however, virtual: the valves are never fully extended or retracted, at least more than momentarily, since either heart allowed to beat with no load for more than a short time is injurious.

In many cases, replacement hearts will have been secondarily impaired as the result of a primary kidney, liver, pulmonary, or gastrointestinal disorder. The same pertains to any type organ available for transplantation. In many cases, replacement hearts will have been secondarily impaired as the result of a primary disorder of the donor, often metabolic, most often involving the kidneys, liver, lungs, or gut The same pertains to any type organ available for transplantation. Unless the recipient suffers from the same disorder, extricating the graft organ, here, a replacement heart, from this toxic setting in itself relieves the graft heart of this chronic insult.

As delineated here, the donor maintained on life support is treated to ameliorate to the extent possible, the condition or conditions that militate against graft organ sufficiency, and treatment of the donor assumes the suppression of any such condition as a matter of principle. Also favorable to both hearts is the reduction in load, roughly to half, and critically, that each is supported by implanted control system, which can target medication to or adjust the load on either. Releasing drugs to include inotropes, and mild electrostimulation elevated to shock level automatically only when an arrest would appear imminent, the prospect for at least the partial recovery of both would seem reasonable.

Toxic environments comprehend numerous metabolic and biochemical disorders that affect the entire body or differentially affect the graft organ or a combination of organs, glands, and/or tissues. Where the heart has been affected secondarily due to a primary disorder elsewhere in the body—the liver, kidney, or lungs, for example—removing the heart from this environment and placing it in another not so affected with the application of suitable therapy as indicated would reasonably allow both hearts to recover.

Because most recipients of a replacement or additional heart due to heart failure are also likely to present comorbidities, the probability is that an alternative or overlapping set of disorders would be encountered along with the stress of the procedure and the immune response; however, these are likely to differently affect the graft organ, affording a measure of relief from the previous setting, and the immediacy and direct targeting of drugs by the automatic response system to suppress adverse symptoms offers an opportunity for systemic recovery. Whether either the superior or inferior vena cava needs to be toggled or both or these states alternated warrants study in either of both hearts. Toggling is highly speculative and may not be necessary.

Based upon the azygos factor, a reduction by half the volume of venous return with the valves set at the halfway point, that is, reduction by about forty percent relative to a midpoint setting, through the venae cavae of one of the hearts should sustain normal function in the one heart during which the other heart is allowed ninety percent of the normal volume, or an increase in forty percent relative to the midpoint setting. Here the situation is complicated by the fact that venous return before the reduction in flow had already been reduced to about half with no opportunity for the heart to have adapted, but assuming these factors are limiting is speculative. On the ejection side, the switching between the hearts of systoles in close succession effectively achieves an ejection fraction within the normal range.

Primary disorders that secondarily involve the heart constitute numerous syndromes to include renocardiac, hepatocardiac, hypertensive, diabetic, hepatopulmonary, portopulmonary hypertensive, and so on (see, for example, Tourki, B., Kain, V., Pullen, A. B., Norris, P. C., Patel, N., and 4 others 2020. “Lack of Resolution Sensor Drives Age-related Cardiometabolic and Cardiorenal Defects and Impedes Inflammation-resolution in Heart Failure,” Molecular Metabolism 31:138-149; Chen, Y. C., Hsu, P. Y., Hsiao, C. C., and Lin, M. C. 2019. “Epigenetics: A Potential Mechanism Involved in the Pathogenesis of Various Adverse Consequences of Obstructive Sleep Apnea,” Online, MDPI [Multidisciplinary Digital Publishing Institute] International Journal of Molecular Sciences (Basel, Switzerland) 20(12). pii: E2937; Kelly, L., Matsumoto, C. L., Schreiber, Y., Gordon, J., Willms, H., and 4 others 2019. “Prevalence of Chronic Kidney Disease and Cardiovascular Comorbidities in Adults in First Nations Communities in Northwest Ontario: A Retrospective Observational Study,” Canadian Medical Association Open 7(3):E568-E572; Kumar, U., Wettersten, N., and Garimella, P. S. 2019. “Cardiorenal Syndrome: Pathophysiology,” Cardiology Clinics 37(3):251-265; Portincasa, P. 2019. “The Two Congested Failing Giants: Heart and Liver,” Internal and Emergency Medicine 14(6):907-910; Correale, M., Tarantino, N., Petrucci, R., Tricarico, L., Laonigro, I., Di Biase, M., and Brunetti, N. D. 2018. “Liver Disease and Heart Failure: Back and Forth,” European Journal of Internal Medicine 48:25-34; Halade, G. V., Kain, V., and Ingle, K. A. 2018. “Heart Functional and Structural Compendium of Cardiosplenic and Cardiorenal Networks in Acute and Chronic Heart Failure Pathology,” American Journal of Physiology. Heart and Circulatory Physiology 314(2):H255-H267; Kieswich, J. E., Chen, J., Alliouachene, S., Caton, P. W., McCafferty, K., Thiemermann, C., and Yaqoob, M. M. 2018. “A Novel Model of Reno-cardiac Syndrome in the C57BL/6 Mouse Strain,” Online, BioMed Central Nephrology 19(1):346; see also, jax.org/news-and-insights/jax-blog/2016/june/there-is-no-such-thing-as-a-b6-mouse]; Virzì, G. M., Clementi, A3., Brocca, A., de Cal, M., and Ronco, C. 2018. “Epigenetics: A Potential Key Mechanism Involved in the Pathogenesis of Cardiorenal Syndromes,” Journal of Nephrology 31(3):333-341; Kingma, J. G., Simard, D., Rouleau, J. R., Drolet, B., and Simard, C. 2017. “The Physiopathology of Cardiorenal Syndrome: A Review of the Potential Contributions of Inflammation,” Journal of Cardiovascular Development and Disease (Basel, Switzerland) 4(4). pii: E21; Horvatits, T., Drolz, A., Rutter, K., Roedl, K., Kluge, S., and Fuhrmann, V. 2016. “Hepatocardiac Disorders: Interactions between Two Organ Systems,” (in German with abstract at Pubmed), Medizinische Klinik, Intensivmedizin, und Notfallmedizin 111(5):447-452; Kingma, J. G. Jr., Simard, D., and Rouleau, J. R. 2015. “Renocardiac Syndromes: Physiopathology and Treatment Stratagems,” Canadian Journal of Kidney Health and Disease 2:41; Fouad, Y. M. and Yehia, R. 2014. “Hepato-Cardiac Disorders,” World Journal of Hepatology 6(1):41-54; Liu, S., Licata, A., Mazzola, A., Ingrassia, D., Calvaruso, V., Camma, C., and Craxi, A. 2014. “Clinical Implications of the Hyperdynamic Syndrome in Cirrhosis,” European Journal of Internal Medicine 25(9):795-802; Kompa, A. R., Kumfu, S., Nishijima, F., Kelly, D. J., Krum, H., and Wang, B. H. 2013. “Subtotal Nephrectomy Accelerates Pathological Cardiac Remodeling Post-myocardial Infarction: Implications for Cardiorenal Syndrome,” International Journal of Cardiology 168(3):1866-1880; Rubattu, S., Mennuni, S., Testa, M., Mennuni, M., Pierelli, G., and 5 others 2013. “Pathogenesis of Chronic Cardiorenal Syndrome: Is There a Role for Oxidative Stress?,” Online, MDPI [Multidisciplinary Digital Publishing Institute] International Journal of Molecular Sciences (Basel, Switzerland) 14(11):23011-23032; Napoli, C., Casamassimi, A., Crudele, V., Infante, T., and Abbondanza, C. 2011. “Kidney and Heart Interactions during Cardiorenal Syndrome: A Molecular and Clinical Pathogenic Framework,” Future Cardiology 7(4):485-497; Naschitz, J. E., Slobodin, G., Lewis, R. J., Zuckerman, E., and Yeshurun, D. 2000. “Heart Diseases Affecting the Liver and Liver Diseases Affecting the Heart,” American Heart Journal 140(1):111-120; additional references to combination syndromes is provided above in section 2a).

The subject is therefore broached as conceivably boding useful potential: for example, when a second heart is added temporarily so that the workload on each is reduced and both supported medically, both can be expected to recover so that either will become available for transplantation or retransplantation. Requiring a second operation—open chest if the heart to be harvested has been placed in the chest—such a contingency is reserved for only the most exigent circumstances, the point made to stress that in vivo restoration to the point of independent sufficiency is possible.

However, as long as both hearts remain in place, the extent of restoration is not likely to exceed that needed to adapt to and optimize circulatory function for the existing configuration, improvement then continued in the recipient. This factor may limit the population of prospective recipients or impose therapeutic measures to sustain the new heart until such support is no longer needed. One eventuality is that a second heart is placed to forestall removal of that native.

That the second if not both hearts were likely harvested from donors who had suffered disorders in which the heard had been affected secondarily or in parallel with one or more other organs means that removal from that toxic milieu should afford it the opportunity to recover, the complications associated with transplantation notwithstanding. Not only do mechanical assist devices lack the potential to provide a second heart which if imperative, remains available to save another patient, but these pose complications.

Both the earlier pulsatile and more recent continuous flow devices impose drawbacks even when used to support one heart (see, for example, Figueiredo, C., Eicke, D., Yuzefovych, Y., Avsar, M., Hanke, J. S., and 5 others 2019. “Low Immunogenic Endothelial Cells Endothelialize the Left Ventricular Assist Device,” (Nature Research) Scientific Reports 9(1):11318; Patel, S., Rizvi, S. S. A., Choi, J. H., Horan, D. P., Weber, M. P., and 7 others 2019. “Management and Outcomes of Left Ventricular Assist Device-associated Endocarditis: A Systematic Review,” Annals of Cardiothoracic Surgery (Hong Kong, China) 8(6):600-609; Cheng, A., Williamitis, C. A., and Slaughter, M. S. 2014. “Comparison of Continuous-flow and Pulsatile-flow Left Ventricular Assist Devices: Is There an Advantage to Pulsatility?,” Annals of Cardiothoracic Surgery (Hong Kong, China) 3(6):573-581).

While extended implantation of an assist device may foster recovery of a failing heart at which time the assist can be removed (Phan, K., Huo, Y. R., Zhao, D. F., Yan, T. D., and Tchantchaleishvili, V. 2016. “Ventricular Recovery and Pump Explantation in Patients Supported by Left Ventricular Assist Devices: A Systematic Review,” American Society for Artificial Internal Organs Journal 62(3):219-231), often the improvement proves temporary (Marinescu, K. K., Uriel, N., Mann, D. L., and Burkhoff, D. 2017. “Left Ventricular Assist Device-induced Reverse Remodeling: It's Not Just About Myocardial Recovery,” Expert Review of Medical Devices 14(1):15-26) the device in retrospect having then served as a bridge to transplantation (Ono, M., Nishimura, T., Kinoshita, O., Shiga, T., Kinugawa, K., Nagai, R., and Kyo, S. 2013. “Improved Survival in Patients with Continuous-flow Ventricular Assist Device for Bridge to Heart Transplantation,” Transplantation Proceedings 45(5):2017-2018). No less important, when permanent, the novel arrangement should allow the use of marginal hearts and thus reduce the critical shortage of hearts available for transplantation.

Thus, an advantage of double heart transplantation is that while the ejection fraction of each heart affected by failure with reduced ejection fraction alone is less than normal, the combined and time-coordinated output of the hearts, each driving about half the normal volume of blood, adjustable to favor either, can be continuously varied automatically in response to data sent to the controller from implanted sensors. This reduction in workload, supported by the automatic targeting of drugs, should allow the hearts the opportunity to recover.

Addressed above in section 2b(b)(b), entitled Metered compound vascular bypass, or sudden switched-organ transplantation, vascular servovalves allow extracardiac heart transplantation so that perfusion is never interrupted and the gradual blending of donor and recipient blood afford a measure of immune tolerance induction integral in the process of organ transfer. Sudden compound vascular bypass, or sudden switched-organ transplantation cannot be accomplished without the use of vascular valves, nor can metered compound vascular bypass, or sudden switched-organ transplantation be accomplished without vascular servovalves.

Sensors placed in the recipient anticipate complications with the specific patient based on the medical records. Using vascular servovalves, the diversion chutes can be adjusted in extension of retraction, so that the process of organ transfer from the donor to the recipient is continuously monitored and automatically controlled to adjust the rate of reciprocal cross-circulation, hence the rate of organ transfer making it possible to avoid serious complications secondary to known medical conditions such as pulmonary hypertension, for example (Klotz, S., Wenzelburger, F., Stypmann, J., Welp, H., Drees, G., Schmid, C., and Scheld, H. H. 2006. “Reversible Pulmonary hHypertension in Heart Transplant Candidates: To Transplant or Not to Transplant,” Online, Annals of Thoracic Surgery (Amsterdam, North Holland, Netherlands) 82(5):1770-1773).

The control system can reduce, maintain, or increase the rate, suspend the action, or if necessary, end it. Metered Compound vascular bypass, or sudden switched-organ Transplantation allows the integration into the process of organ transfer of gene as well as stem cell transfer both for restorative and immune tolerance induction to augment that inherently provided by the reciprocal cross-microchimerization that results from reciprocal cross-circulation. Also addressed there is the considerable expansion in the pool of organs to include ordinarily considered marginal or unqualified, such as subcritically failing hearts, that will have become acceptable for transplantation.

Since the mainlines, or bloodlines, and servovalves remain in place once the recipient has been closed, the valves can be automatically adjusted by the implanted master controller microprocessor or microcontroller that administered the transplantation to alter the rate of venous return and directly pipeline target medication, whether immunosuppressant, anti-inflammatory, or antimicrobial, for example, through the accessory channel or channels of each valve into the bloodstream. Such automatic response to immediate conditions as reported to the controller by implanted sensors is executed by an automatic disorder response system described in U.S. Pat. Nos. 11,013,858 and 11,759,186.

The implanted microcontroller can also actuate in situ electrical discharge or warming implants according to its response prescription-program. These features allow the placement of a heart that would ordinarily be rejected as marginal. Further expansion in the zone of hearts made usable through the intercession of such an implanted automatic response system is provided by double heart transplantation. Conventional heterotopic heart transplantation essentially replaces the native ventricles of a heart which decompensated, has become incapable of providing adequate absolute stroke volume, and/or is too weakened to offset the pulmonary vascular resistance it confronts.

In contrast, double heart heterotopic transplantation allows the addition of two marginal ventricles to those native, to achieve adequacy of absolute stroke volume of the two hearts working as a pair. By allowing the use of many hearts that would otherwise be rejected for transplantation, waiting times can be considerably reduced. Moreover, the implanted automatic disorder response system continuously monitors and coordinates the action of each heart as well as that of the two acting as a unit. The automatic therapeutic support provided to each heart and the mutual support each provides to the other afford both the opportunity to regain lost sufficiency. However, any hypertension the recovery of either or both might produce is readily offset by the system, which is provided with multiple means, mechanical and medicinal, for diverting or clutching blood flow, and thus defraying or suppressing local hypertension.

Of the deterrents respective of heart assist devices and a heart transplant, that most prominent of mechanical devices-ventricular assist devices and artificial hearts—is the need to supply adequate power, while that of transplantation is the prospect of rejection and the need to undergo a second operation to explant the transplant for replacement with an artificial heart or by retransplantation. Whether due to defects in the heart of a neonate too severe to impart normal circulation through surgical repair without assistance or to advanced stage heart failure, the addition of a second heart by double heart metered compound vascular bypass, or sudden switched-organ transplantation as described herein is able to compensate for the inadequacy of the native heart and impart satisfactory perfusion. In a transplantation context, the terms ‘explant’ and ‘explantation’ are preferably limited to the removal of implants, not to the harvesting of native anatomy.

In both a neonate with an unsurvivably malformed heart and an adult with end stage heart failure, a double heart transplant wherein each heart need not be perfect substantially expands the pool of hearts available for transplant. In so doing, patients who would die while waiting for a strong replacement heart became available would survive. Each heart intact and positioned separately from that native, a double heart transplant is superior to either mechanical devices or a single less than perfect heart placed orthotopically. Whether used to replace a severely defective heart in the very young or very old, double heart switch transplantation contemplates that since the absolute stroke volume of the native heart is inadequate, the addition of a second heart with marginal ventricles is not likely to result in a joint stroke volume much greater than normal.

The larger number of hearts which the loosened criteria make available increase the likelihood that one of these will complement the native heart. If none is available, then based upon sensor feedback, the implanted system can directly target either or both hearts to intensify or relax ejection through electrostimulation of either conduction system or targeting positive or negative inotropes to either or both hearts. The system can therefore balance the action of the hearts to function optimally as a unit and return the hearts to the proper setpoints should either or both deviate from these values. That the support of either heart by another might allow both to regain in strength is briefly addressed above under 1. Background of the Invention, a. General. To the improbability that the absolute stroke volume would then burgeon may be added that even if it did, the end condition would be kept within limits throughout.

The probability is that any implant to assist or replace the native heart will eventually require to be replaced; however, with current immunosuppressant measures, two organic hearts working in coordination and continuously supported by an automatic system that monitors and instantly responds to the need for pharmaceutical or electrostimulatory assistance can remain sufficiently competent for many years longer and with less likelihood of malfunctioning than might any solitary compromised heart of mechanical device. That the second heart need not be robust considerably expands the zone of usable hearts, and the cost of the procedure is less than that for any mechanical device.

Ordinarily, each heart is functionally optimized itself and as functionally unified with the other. While an orthotopic transplant is solitary without backup, in a double heart transplant, should either heart show signs of imminent or established failure evidenced by a lack of response to treatment, the implanted control system concentrates the therapeutic pipeline targeting of remedial drugs to that heart, and if no improvement results, cuts off venous return to that heart, and moves all support, pharmaceutical and electrostimulatory as appropriate, to the other heart. That is, the automatic system will either bypass the native heart or close off the bypass through the transplant. Moreover, if during the interval since transplantation, the remaining heart has recovered sufficiently, the failed heart is removed and the patient closed.

If not the remaining heart should sustain the patient long enough to undergo the placement of a second heart, an orthotopic retransplantation, or the placement of a mechanical device as a bridge to retransplantation. Unless severely impaired, living hearts have no need for exogenous power or assistance, and with the pipeline targeted administration of immunosuppressant medication as addressed and illustrated in U.S. Pat. No. 11,759,186, a second heart separate from that native, can be immunologically supported with negligible if any further exposure of nontargeted tissue to the medication and the inducement of adverse side effects. The automatic administration of medication assures compliance in the very young, the very old, those with dementia, or presenting mental or learning disorders.

Separate positioning of the two hearts facilitates the ability to target chemotherapeutic drugs and radionuclides to each or to a circumscribed location surrounding each, or to focus external beam radiation to only one of two hearts, and in so doing, avoid harmful side effects. It also facilitates the resection or removal of either heart should malignancy require such treatment. Isolated removal is not practical with a conventional heterotopic or ‘piggyback’ transplant, and no other starting condition leaves behind a functioning heart to sustain the patient after the cancerous or otherwise diseased heart has been disabled or removed.

While primary benign tumors and malignancies of the myocardium are rare, this is not true of secondary malignancy due to metastasis to the native heart from the pericardium, the tissues surrounding the heart, or ‘daughter cells’ shed by a separately located primary malignancy (see, for example, Bonou, M., Kapelios, C. J., Marinakos, A., Adamopoulos, S., Diamantopoulos, P., and 5 others 2019. “Diagnosis and Treatment Complications of Primary Cardiac Lymphoma in an Immunocompetent 28-year Old Man: A Case Report,” Online, BioMed Central Cancer 19(1): 191; Gioia, M., Alawieh. H., Amoun, T., Bangash. A., Horgan, S., Uretsky, S., and Safirstein, J. 2019. “A Rare Case of Primary Intracardiac Diffuse Large B-cell Lymphoma: Multimodality Imaging to the Rescue,” Cardiovascular Imaging Case Reports (Philadelphia, Pennsylvania) 3(3):133-136; Martinez-Giron, R., Pantanowitz, L., Martinez-Torre, S., and Pantanowitz, J. 2019. “Sudden Cardiac Death Due to Primary Malignant Pericardial Mesothelioma: Brief Report and Literature Review,” Respiratory Medicine Case Reports 26:185-188; Sun, W. Z., Wu, Q. J., Wang, Y., Chen, M., Zhang, J., Zhang, G., and Zhou, Y. F. 2019. “A Rare Case of Primary Well-differentiated Angiosarcoma of the Right Atrium,” Journal of Geriatric Cardiology 16(2):164-167; Yu, J. F., Cui, H., Ji. G. M., Li, S. Q., Huang, Y., Wang, R. N., and Xiao, W. F. 2019. “Clinical and Imaging Manifestations of Primary Cardiac Angiosarcoma,” Online, BioMed Central Medical Imaging 19(1):16; Guazzaroni, M., Lacchè, A., Nardone, V., Garipoli, A., Pizzicannella, G., and 3 others 2018. “Evaluation of a Cardiac Sarcoma with CT Multislice Contrast-enhanced and 18FDG-PET/TC [positron emission tomography with fluorodeoxyglucose, or 2-deoxy-2-[fluorine-18] fluoro-D-glucose integrated with computed tomography],” Radiology Case Reports 14(3): 368-371; Liu, C., Zhao, Y., Yin, Z., Hu. T., Ren. J., and 6 others 2018. “Right Atrial Epithelioid Angiosarcoma with Multiple Pulmonary Metastasis Confirmed by Multimodality Imaging-guided Pulmonary Biopsy: A Case Report and Literature Review,” Medicine (Baltimore, Maryland) 97(30):e11588; Wang, S., Li, M., Zhang, L., and Xie, M. 2018. Multimodal Imaging Evaluation of a Primary Cardiac Lymphoma in an Immunocompetent Patient,” Echocardiography 35(12):2121-2123; Ciancarella, P., Fusco, A., Citraro, D., Sperandio, M., and Floris. R. 2017. “Multimodality Imaging Evaluation of a Primary Cardiac Lymphoma,” Journal of the Saudi Heart Association 29(2):128-135; Katinaite, J. and Petrauskienė, B. 2017. “Recurrent Pericarditis: A Case Report and Literature Review,” Acta Medica Lituanica (Vilnius, Lithuania) 24(3):159-166; Martinez, C., Rana, J. S., and Solomon, M. D. 2017. “Cardiac Metastasis of Nonvisceral Soft-tissue Leiomyosarcoma,” Reviews in Cardiovascular Medicine 18(2):78-81; Rao, J. N., Gowda, G. D., Anand. R., and Desai, N. B. 2017. “Angiosarcoma of the Right Atrium and Right Ventricle,” Journal of Cardiac Surgery 32(12):807-808; Zhang, R., Li, L., Li, X., Chu, Y., Wu. J., and Gao, Y. 2017. “Primary Cardiac Angiosarcoma: A Case Report,” Medicine (Baltimore, Maryland) 96(42):e 7352; Mauricio, R., Mgbako, O., Buntaine, A., Moreira. A., and Jung, A. 2016. “Complete Resolution of Tumor Burden of Primary Cardiac Non-Hodgkin's Lymphoma,” Case Report in Cardiology 2016:2124975; Saisho, C., Ishii, H., Edakuni, N., Imamura, Y., Tokito. T., and 4 others 2016. “Sarcomatoid Type Primary Pericardial Mesothelioma with a Long-term Survival after the Onset of Cardiac Tamponade,” Internal Medicine (Tokyo, Japan) 55(21):3161-3164; Jandali, A., Kabach, A., Al Halabi, S., and Alraies, M. C. 2015. “Rare Cause of a Common Symptom: Primary Cardiac Lymphoma,” American Journal of Emergency Medicine 33(12):1849.e5-e6; Montanaro, C., Loiacono, F., Fragasso, G., De Cobelli, F., Foppoli, M., and Margonato, A. 2015. “Primary Cardiac Lymphoma in an Immunocompetent 71-year-old Man,” Texas Heart Institute Journal 42(6):561-564; Pistritto, A. M., Pavo, N., Maurer, G., Binder, T., and Goliasch, G. 2015. “Multimodality Imaging of a Primary Cardiac Diffuse Large B-cell Lymphoma,” European Heart Journal Cardiovascular Imaging 16(8):909; Burazor, I., Aviel-Ronen, S., Imazio, M., Markel, G., Grossman, Y., Yosepovich, A., and Adler, Y. 2014. “Primary Malignancies of the Heart and Pericardium,” Clinical Cardiology 37(9):582-588; Nijjar, P. S., Masri, S. C., Tamene, A., Kassahun, H., Liao, K., and Valeti, U. 2014. “Benefits and Limitations of Multimodality Imaging in the Diagnosis of a Primary Cardiac Lymphoma,” Texas Heart Institute Journal 41(6):657-659; Sardar, M. R., Kuntz, C., Patel, T., Saeed, W., Gnall, E., Imaizumi, S., and Lande, L. 2012. “Primary Pericardial Mesothelioma Unique Case and Literature Review,” Texas Heart Institute Journal 39(2):261-264; Leja, M. J., Shah, D. J., and Reardon, M. J. 2011. “Primary Cardiac Tumors,” Texas Heart Institute Journal 38(3):261-262; Masroor, S. and Marla, R. 2011. “Left Atrial Angiosarcoma: An Unusual Presentation and Location,” Journal of Heart Valve Disease 20(2):229-231; Brinckman, S. L. and van der Wouw, P. 2005. “Images in Cardiovascular Medicine. Angiosarcoma of the Pericardium: A Fatal Disease,” Circulation 111(23):e388-e389; Lee, C. H., Chan, G. S. W., and Chan, W. M. 2003. “Unexplained Recurrent Pericardial Effusion: A Lethal Warning?,” Heart 89(3):e11).

The development of cancer following a heart transplant is common but does not appear to apply to the transplant but rather other organs and tissues. That without direct pipeline targeting of immunosuppressants, these other organs and tissues may be made more susceptible to malignancy is likely involved. Where an automatic transplant monitoring and support system is fully implanted, relegating the results of the operation to the out of sight, out of mind, an assist device or artificial heart imposes the need to wear power and control packs constantly in sight and in mind or hidden beneath apparel. In a child, a conventional orthotopic or an orthotopic double heart transplant as described herein will grow with the child and last many years. With further research, it will last to the end of life with minimal upkeep and minimal distraction from artificial accoutrements requiring maintenance.

While in most cases a second heart affords that native heart the opportunity to recover to an extent, in all cases, it can sustain the patient should the native heart become further impaired. The native heart can then be removed, and if necessary, replaced. If the added heart becomes impaired for any reason, should it become infected due to immunosuppression, for example, the same remedy is available. While the impact of cytomegalovirus infection appears less with antiviral prophylaxis, this speaks to only one of numerous infectious pathogens to which the recipient of a heart transplant might be exposed. With the zero ischemic time method of switch transplantation, the incidence of post-transplantation rejection and late-term vasculopathy should be reduced.

Double heart transplantation, metered switch with implanted automatic disorder support system in particular, is distinguished from conventional heterotopic or ‘piggyback’ heart transplantation in two primary regards: The first is that the two hearts, that orthotopic, meaning that native or orthotopically or heterotopically replaced earlier, and the second, added, heart, remain intact. Absent the need for endoscopic or open field entry to accomplish an internal repair, neither heart is incised into. The second distinction is that the two hearts remain separated for targeted diagnostic and/or therapeutic access by a clinician and the implanted automatic disorder monitoring and response system as described in U.S. Pat. No. 11,759,186, entitled Ductus Side-entry Jackets and Prosthetic Disorder Response System.

That the hearts remain separated, one in the chest and the other positioned infrarenally within the abdomen, or within the pelvis, means that either can be directly pipeline targeted for the delivery of medication independently of the other or—entry into the systemic circulation medically insignificant if any and provided a reversal agent is available, reversible if necessary—any other tissue and that if necessary, either can be removed and replaced without the extensive dissection necessary to disengage the two hearts in a conventional heterotopic transplant, of which the surgical trauma involved equals if not exceeds that of having ‘piggyback’-merged the two in the first place.

When a switch heart transplant is initiated, the hearts of both donor and recipient each drive all the blood in their respective circulatory systems. Throughout the transfer of the donor heart from the circulatory system of the donor to that of the recipient, the volume handled by each heart remains the same but the relative proportion of blood in either circulatory system from the other party is gradually increased. Upon completion of the transfer, the heart of the donor is closed off from its respective circulatory system but contains the normal volume of blood which is added to the total volume in the recipient. This increment in total blood volume, about one third of the total, reduces the potential hypovolemia that might otherwise arise.

Normally, the intracardiac, or stressed volume, represents about one third of the total system volume. This addition of about one third more blood into the recipient circulatory system serves to reduce a hypovolemia as experienced by two hearts driving one and one third, rather than twice, the normal volume of blood. This condition of two hearts driving one circulatory system is known from the experimental transplantation into the abdomens of rats of a second heart for unrelated experimentation, usually immunological, where the volumetric discrepancy is even greater, the added heart not having brought a heartful of blood with it. Positioning a second heart at a remote location such as in the abdomen without the support of blood tap servovalves and servochokes to alleviate out of range blood pressure valves is as futile as ignoring proper matching and the use of immunosuppressives.

Side-entry vascular connection devices described in related applications specified above in section 2a make possible the heterotopic positioning of accessory organs at a greater than usual distance from that orthotopic; however, to do so is discouraged as engaging excessive risk and complexity. While the hemopoietic system adapts to changes in volume such as following a loss of blood, the need for half again the volume of blood during pregnancy, as seen in the azygos factor, which can sustain the heart after venous return has been severely impaired; and in that heart adapts to changes in vascular resistance, for example, the question still remains whether following an interval to allow adaptation to the postoperative condition, intracardiac hypovolemia will have been dispelled or continue to pose a risk of intracardiac dysrhythmia.

Here a scheme for toggling between half and full venous retum and stroke volume of either heart at a rate determined by a test algorithm is provided to support the transplantation postoperatively during adaptation and thereafter dispel the eventuality that chronic hypovolemia will pose a problem. If the native and added hearts are the same in size absolute stroke volume, the setpoint about which the diversion valves are toggled is at the halfway point of overall diversion chute throw or stroke, and this is adjusted for inequality between the hearts. Earlier schemes for double heart transplantation usually connected the second heart in series with the native heart while empty of blood so that each heart moved the volume of blood in the recipient circulatory system in sequence.

In a common method used more recently to create a rat model for studying unloading-induced myocardial remodeling, but historically, for immunological and pharmacological experimentation, the aorta and pulmonary artery of the donor heart are anastomosed directly to the abdominal aorta and inferior vena cava of the recipient, since modified by Didie (Didie, M., Biermann, D., Buchert, R., Hess. A., Wittkopper, K., and 7 others 2013. “Preservation of Left Ventricular Function and Morphology in Volume-loaded versus Volume-unloaded Heterotopic Heart Transplants,” American Journal of Physiology. Heart and Circulatory Physiology 305(4):H533-H541).

These relatively short-term experiments, until recently not directed to cardiac physiology or the development of a sound means for long-term transplantation of the heart, but rather to resolving wider questions of immunology and pharmacology, are of no value for predicting the consequences of metered switch double heart transplantation, where working together, both hearts must remain competent for years. In these experiments, the added heart contributed nothing to the circulatory sufficiency of the recipient, which was normal to begin with.

Even though some blood entered the second from the inferior vena cava to be ejected by the left ventricle into the abdominal aorta, slight effort was involved, so that a nonfunctional transplant, the second heart underwent disuse atrophy. Such a nonfunctional transplant is fundamentally unlike a double heart transplant, in which the hearts are intimately connected in series and work in a cooperative manner so that neither is subject to disuse. Conventional heterotopic transplantation and the use of a ventricular assist device both pose the ‘double-edged sword’ benefit of reverse remodeling along with the risk of disuse atrophy.

“Following heterotopic transplantation, the rat heart undergoes atrophy and exhibits delayed cardiac relaxation without any changes in contraction and systolic Ca 2+ transients. Furthermore, the sarcoplasmic reticular Ca 2+ uptake and release activities were reduced and Ca 2+ influx through L-type Ca 2+ channels was increased in the atrophied heart,” (Makino, N., Ganguly, P., Elimban, V., and Dhalla, N. S. 2018. “Sarcolemmal Alterations in Unloaded Rat Heart after Heterotopic Transplantation,” International Journal of Angiology 27(4):196-201)(see also Eisen, H. J. 2019. “Left Ventricular Assist Devices (LVADS): History, Clinical Application, and Complications,” Korean Circulation Journal 49(7):568-585; Mccullough, M., Caraballo, C., Ravindra, N. G., Miller, P. E., Mezzacappa. C., and 8 others 2019. “Neurohormonal Blockade and Clinical Outcomes in Patients with Heart Failure Supported by Left Ventricular Assist Devices,” Journal of the American Medical Association Cardiology November 18; Shugh, S. B., Riggs, K. W., and Morales, D. L. S. 2019. “Mechanical Circulatory Support in Children: Past, Present, and Future,” Translational Pediatrics 8(4):269-277; Westhofen, S., Jelinek, M., Dreher, L., Biermann, D., Martin, J., and 4 others 2019. “The Heterotopic Heart Transplantation in Mice as a Small Animal Model to Study Mechanical Unloading-Establishment of the Procedure, Perioperative Management and Postoperative Scoring,” Online, PLoS [Public Library of Science] One 14(4):e0214513; Pokomy M., Mrazova, I., Šochman, J., Melenovsky. V., Maly, J., and 9 others 2018. “Isovolumic Loading of the Failing Heart by Intraventricular Placement of a Spring Expander Attenuates Cardiac Atrophy after Heterotopic Heart Transplantation,” Bioscience Reports 38(3). pii: BSR20180371; Wright, P. T., Sanchez-Alonso, J. L., Lucarelli, C., Alvarez-Laviada, A., Poulet, C. E., and 4 others 2018. “Partial Mechanical Unloading of the Heart Disrupts L-type Calcium Channel and Beta-Adrenoceptor Signaling Microdomains,” Online, Frontiers in Physiology 9:1302; Benke, K., Sayour, A. A., Mátyás. C., Agg. B., Németh, B. T., and 7 others 2017. “Heterotopic Abdominal Rat Heart Transplantation as a Model to Investigate Volume Dependency of Myocardial Remodeling,” Transplantation 101(3):498-505: Vaijyanath, P. 2017. “A New Technique of Heterotopic Heart Transplantation with Direct Pulmonary Artery Anastomosis,” Journal of the Tehran Heart Center 12(4):194-195; Drakos, S. G. and Mehra. M. R. 2016. “Clinical Myocardial Recovery during Long-term Mechanical Support in Advanced Heart Failure: Insights into Moving the Field Forward” Journal of Heart and Lung Transplantation (New York, New York) 35(4):413-420; Fu, X., Segiser, A., Carrel, T. P., Tevaearai Stahel, H. T., and Most. H. 2016. “Rat Heterotopic Heart Transplantation Model to Investigate Unloading-induced Myocardial Remodeling,” Online, Frontiers in Cardiovascular Medicine 3:34; Książek, A. A., Mitchell, K. J., Morax, L., Schwarzwald, C. C., Hoerstrup, S. P., and Weber, B. 2016. “Hemodynamic Assessment of a Murine Heterotopic Biventricularly Loaded Cardiac Transplant in Vivo Model,” European Surgical Research 57(3-4):171-185; Liu, Y., Maureira, P., Gauchotte. G., Falanga, A., Marie. V., and 5 others 2015. “Effect of Chronic Left Ventricular Unloading on Myocardial Remodeling: Multimodal Assessment of Two Heterotopic Heart Transplantation Techniques,” Journal of Heart and Lung Transplantation (New York, New York) 34(4):594-603; Lu, W., Zheng. J., Pan, X. D., Li, B., Zhang, J. W., Wang, L. F., and Sun, L. Z. 2015. “A New Simplified Volume-loaded Heterotopic Rabbit Heart Transplant Model with Improved Techniques and a Standard Operating Procedure,” Journal of Thoracic Disease 7(4):653-661; Oriyanhan, W., D'Alessandro, D., Bello, R., Xia, Y., Follis, M., Moreno, G., and Michler, R. 2015. “A Modified Biventricular Working Heterotopic Rat Heart Transplant Model with Pressure Volume Loops Cardiac Function Analysis,” Journal of Heart and Lung Transplantation (New York, New York) 34(4):Supplement S267; Jahanyar, J., Koerner, M. M., Ghodsizad, A., Loebe, M., and Noon, G. P. 2014. “Heterotopic Heart Transplantation: The United States Experience,” Heart Surgery Foran 17(3):E132-E140; Ibrahim, M., Navaratnarajah, M., Kukadia, P., Rao. C., Siedlecka, U., and 5 others 2013. “Heterotopic Abdominal Heart Transplantation in Rats for Functional Studies of Ventricular Unloading,” Journal of Surgical Research 179(1):e31-e39; Wen, P., Wang. X., Li, T., Zhang, B., Sun, X., and 6 others 2013. “Establishment of a Novel Volume-loaded Heterotopic Heart Transplantation Model in Rats,” Journal of Surgical Research 183(1):435- 441: Wen, P., Wang, X., Wang, J., Zhang, B., Qiu, G., and 5 others 2013. “A Simple Technique for a New Working Heterotopic Heart Transplantation Model in Rats,” Transplantation Proceedings 45(6):2522-2526; Tang-Quan, K. R., Bartos, J., Deuse. T., Churchill, E., Schafer, H., and 4 others 2010. “Non-volume-loaded Heart Provides a More Relevant Heterotopic Transplantation Model,” Transplant Immunology 23(1-2):65-70; Malliaras, K. G., Terrovitis, J. V., Drakos, S. G., and Nanas, J. N. 2009. “Reverse Cardiac Remodeling Enabled by Mechanical Unloading of the Left Ventricle,” Journal of Cardiovascular Translational Research 2(1):114-125; Gordon, C. R., Matthews, M. S., Lefebvre, D. R., Strande, L. F., Marra, S. W., and 3 others 2007. “A New Modified Technique for Heterotopic Femoral Heart Transplantation in Rats,” Journal of Surgical Research 139(2):157-163; Kadner, A., Chen, R. H., and Adams, D. H. 2000, Op cit. section 2b(1)(b)(b3); Asfour, B., Hare, J. M., Kohl, T., Baba, H. A., Kass. D. A., and 7 others 1999. “A Simple New Model of Physiologically Working Heterotopic Rat Heart Transplantation Provides Hemodynamic Performance Equivalent to that of an Orthotopic Heart,” Journal of Heart and Lung Transplantation (New York, New York) 18(10):927-936; Yokoyama, H., Ohmi, M., Murata. S., Nakame. T., Tabayashi, K., and Mohri. H. 1995. “Proposal of a Working Left Heart Model with a Heterotopic Transplantation Technique in Rats,” Journal of Heart and Lung Transplantation (New York, New York) 14(4):706-712; additional references provided below in this section).

Already provided with a competent heart, the addition of a second, unloaded, heart is merely gratuitous and obtrusive, in addition to demanding the immunocompromising of the recipient as a whole. The positioning of the added heart along the great vessels in the abdomen only interposes a gratuitous perturbation in normal flow. In both conventional heterotopic heart and double heart switch transplantation, venous retum and ejection cannot be diverted into an ‘off-line’ gratuitous heart, so that this condition is not present. Were the native heart impaired, a different reaction might be forthcoming. No factor of unloading pertans to either conventional heterotopic or double heart switch transplantation.

Whether recovery of the native heart as results from the use of a left ventricular assist device (Fu, X., Segiser, A., Carrel. T. P., Tevaearai Stahel, H. T., and Most. H. 2016. Op cit., this section) would reinstate atrophy of the gratuitous backup seems likely but requires testing. With either heart able to support the recipient, whether this atrophy is due entirely or mostly to reliance upon only the native heart and disuse of the other or the presence of two separate time-independent and conflicting points of ejection and venous return warrants study.

Where the experimental placement of a second competent heart in the abdomen results in either heart functioning independently of the other, in both conventional heterotopic and double heart switch transplantation, the hearts are essentially locked together in function. With respect to conventional heterotopic transplantation, where ejection and venous return are unitary due to merger of the atria, atrophy of either heart is not mentioned. Also unmentioned is the addition of a competent heart where that native is failing or whether given time, the modified circulatory system overall would adapt to accommodate both hearts. The latter would appear more likely where neither heart was adequate and depended upon the other to achieve circulatory sufficiency.

The central object in metered switch double heart transplantation is to increase the number of hearts available for transplantation by allowing the addition of a second less than robust heart to promote a measure of interdependence between the two. If necessary, with the aid of occasional electrostimulatory synchronization, this should educe adaptation of the system overall. The gradual recovery of either or both hearts over time is addressed below; this eventuality is accommodated by automatic adjustments of the diversion chutes by the implanted disorder response system. Considerable improvement in the native heart would reasonably prompt explantation of the added hear.

Each great vessel in either heart connected to its counterpart by a synthetic mainline, or bloodline, no aorta connecting the two is present to intrinsic timing conflict the hearts eject and receive blood at unitary points of outflow and inflow with respect to the extracardiac circulatory system. Placed in the chest, the added heart is proximate that native in adjacent relation. Moreover, the bloodlines not flexible as are elastic arteries, the sensation of pulsation at the venous return of the upstream heart is negligible even when the second heart is placed within the chest.

Based upon conventional, or ‘piggyback’ heterotopic heart transplantation, the effect of asynchronous systoles between the two left ventricles and segments of the aorta, and based upon intrinsic aorta-heart synchronous contraction in experimental inguinal implantation of a second heart are likely spontaneously adapted to no ill effect (see, for example, Caulk, A. W., Humphrey, J. D., and Murtada, S. I. 2019. “Fundamental Roles of Axial Stretch in Isometric and Isobaric Evaluations of Vascular Contractility,” Journal of Biomechanical Engineering 141(3); Leloup, A. J. A., Van Hove, C. E., De Moudt. S., De Meyer, G. R. Y., De Keulenaer, G. W., and Fransen, P. 2019. “Vascular Smooth Muscle Cell Contraction and Relaxation in the Isolated Aorta: A Critical Regulator of Large Artery Compliance,” Physiological Reports 7(4):e13934; De Moudt. S., Leloup, A., Van Hove, C., De Meyer, G., and Fransen, P. 2017. “Isometric Stretch Alters Vascular Reactivity of Mouse Aortic Segments,” Online, Frontiers in Physiology 8:157; Leloup. A., De Moudt. S., Van Hove. C., and Fransen, P. 2017. “Cyclic Stretch Alters Vascular Reactivity of Mouse Aortic Segments,” Online, Frontiers in Physiology 8:858; Fransen, P., Van Hove, C. E., Leloup, A. J., Martinet, W., De Meyer, G. R., and 3 others 2015. “Dissecting Out the Complex Ca2+-mediated Phenylephrine-induced Contractions of Mouse Aortic Segments,” Online, PLoS [Public Library of Science] One 10(3):e0121634; Leloup, A. J., Van Hove, C. E., Heykers, A., Schrijvers, D. M., De Meyer, G. R., and Fransen, P. 2015. “Elastic and Muscular Arteries Differ in Structure, Basal N O Production, and Voltage-gated Ca(2+)-Channels,” Online, Frontiers in Physiology 6:375; Sahibzada, N., Mangel, A. W., Tatge, J. E., Dretchen, K. L., Franz, M. R., Virmani, R., and Gillis, R. A. 2015. “Rhythmic Aortic Contractions Induced by Electrical Stimulation In Vivo in the Rat,” Online, PLoS [Public Library of Science] One 10(7):e0130255; Marion, S. B. and Mangel. A. W. 2014. “From Depolarization-dependent Contractions in Gastrointestinal Smooth Muscle to Aortic Pulse-synchronized Contractions,” Clinical and Experimental Gastroenterology 7:61-66; Fransen, P., Van Hove, C. E., van Langen, J., Schrijvers, D. M., Martinet, W., De Meyer, G. R., and Bult, H. 2012. “Contribution of Transient and Sustained Calcium Influx, and Sensitization to Depolarization-induced Contractions of the Intact Mouse Aorta,” Online, BioMed Central Physiology 12:9; Mangel. A., Fahim, M., and van Breemen, C. 1981. “Rhythmic Contractile Activity of the in Vivo Rabbit Aorta,” Nature 289(5799):692-694). As does conventional heterotopic heart transplantation, switch transplantation of the heart does not result in the creation of separate segments along one and the same aorta.

Immunosuppressants were lacking, and placement of the second heart in the groin (inguinum) or in the abdomen left it unprotected. Experience with survivors of pentalogy of Cantrell or congenital cardiac exposure syndrome-type ectopia cordis with a heart below the thorax, are essentially bedridden, usually unable to engage even in nonstrenuous walking, much less exertional activities.

The lack of sufficient space in the abdomen causes almost any bodily movement to compress the heart and major vessels, immediately inducing cyanosis and dyspnea, and positioned superficially without bony protection, the heart is immediately vulnerable to injury.

Not the result of a procedure to situate a second heart in the chest or deeply in a protective surround within the abdomen, these congenital malformities present with a form that cannot be allowed to remain (see, for example, Reilly, N. 2015. “This Man Was Born with His Heart Outside His Ribcage,” Metro 25 Oct. 2015, Online at metro.co.uk/2015/10/25/this-man-was-born-with-his-heart- outside-his-ribcage-5461200/; Rivas, A. 2013. “Huang Rongming, Man Born with Heart in Abdomen, Undergoes Corrective Surgery,” Medical Daily, 22 Aug. 2013, Online at medicaldaily.com/huang-rongming-man-born-heart-abdomen-undergoes-corrective-surgery-253579; McQueeney, K. 2013. “Ho Ziliang: Man Lives with His Heart in his Abdomen for 24 Years,” Metro 28 Nov. 2013, Online at metro.co.uk/2013/11/28/ho-zhiliang-heart-beats-below-mans-stomach-for-24-years-4206126/Pito=cbshare).

In contrast, to achieve zero ischemia time, eliminate reperfusion injury, allow hematological immune tolerance induction concurrent with transplantation, and maintain direct connection of each major vessel in either heart to its counterparts in the other heart-all important for graft survival-switch transplantation essentially connects the hearts in series-parallel, so that each heart simultaneously moves half the volume of blood of the recipient plus half the blood that had been in the donor heart when the switch was completed. Reference to “continuous normothermic blood perfusion” in the early heart transplants performed by Zerbini in Sao Paulo Brazil are misleading: perfusion was reinstated only after the donor heart had stopped and the intense immunological reaction thereto allowed to transpire (Stolf, N. A. G. 2017. “History of Heart Transplantation: A Hard and Glorious Journey,” Brazilian Journal of Cardiovascular Surgery 32(5):423-427).

At least intra- and postoperatively, the blending of blood between recipient and donor in metered compound vascular bypass, or sudden switched-organ transplantation comprehends aspects of parabiotic perfusion and tissue exchange investigated seven decades ago (see, for example, Serreze, D. V., Osborne, M. A., Chen, Y. G., Chapman, H. D., Pearson, T., Brehm, M. A., and Greiner, D. L. 2006. “Partial versus Full Allogeneic Hemopoietic Chimerization is a Preferential Means to Inhibit Type 1 Diabetes as the Latter Induces Generalized Immunosuppression,” Op cit. preceding section; Jaroslow, B. N, Baffes, T. G, and Ketola, F. H. 1963. “Attempts to Establish Immune Tolerance by Massive Organ Transplantation,” Transactions of the American Society for Artificial Internal Organs 9:135-1388. Marcus, E., Wong, S. N. & Luisada, A. A. 1953. “Homologous Heart Grafts. I. Technique of Interim Parabiotic Perfusion. II. Transplantation of the Heart in Dogs,” American Medical Association Archives of Surgery 66, 179-191). In point of fact, both sudden and metered switch, no less than conventional solid organ transplantation, can include the preoperative exchange between recipient and donor of blood products and glandular tissue.

The answers are complex and remain in need of clarification (see, for example, Elgenaidi, I. S. and Spiers, J. P. 2019. “Hypoxia Modulates Protein Phosphatase 2A through HIF-1α Dependent and Independent Mechanisms in Human Aortic Smooth Muscle Cells and Ventricular Cardiomyocytes,” British Journal of Pharmacology 176(11):1745-1763; Jacobs, R., Lochy, S., and Malbrain, M. L. N. G. 2019. “Phenylephrine-induced Recruitable Preload from the Venous Side,” Journal of Clinical Monitoring and Computing 33(3):373-376; Sharma, R. and Sharma, S. 2019. “Physiology, Blood Volume,” Treasure Island, Florida: StatPearls Publishing Co., Online at www.ncbi.nlm.nih.gov/books/NBK526077/; Jones, D. G., Nantais, J., Rezende-Neto, J. B., Yazdani, S., Vegas, P., and Rizoli, S. 2018. “Crystalloid Resuscitation in Trauma Patients: Deleterious Effect of 5 L or More in the First 24 h,” Online, BioMed Central Surgery 18(1):93; Magder, S. 2016. “Volume and Its Relationship to Cardiac Output and Venous Retum,” Critical Care 20:271; Wolff, C. B., Collier, D. J., Shah, M., Saxena, M., Brier, T. J., and 3 others 2016. “A Discussion on the Regulation of Blood Flow and Pressure,” Advances in Experimental Medicine and Biology 876:129-135; Lee, S., Willoughby, W. F., Smallwood, C. J., Dawson, A., and Orloff, M. J. 1970, Op cit. above in section 1a; Cooper, D. K. C. 1968. “Experimental Development of Cardiac Transplantation,” Online, British Medical Journal 4(5624):174-181).

Of the sites available for placement of a second intact heart—in the chest, the abdominal cavity, and within the pelvis, that natural, the chest, is preferred (see, for example, Flécher, E., Fouquet, O., Ruggieri, V. G., Chabanne, C., Lelong, B., and Leguerrier, A. 2013. “Heterotopic Heart Transplantation: Where Do We Stand?,” European Journal of Cardiothoracic Surgery 44(2):201-206). The abdomen and infrarenal positions lack the protection afforded by a surrounding bony cage, necessitating the use of an implanted stainless steel heart cage as described below in section 2b(1)(b)(b6), entitled Heart Cage for the Infrarenal Abdominal Positioning of a Second Heart, or an externally worn medieval armor type plackart (planckart, placcard, placcate). Much like the mediastinum, the space available in the pelvis is already accounted for, leaving limited space to fit a second complete beating heart. As in the chest where a portion of the right lung—which regenerates—is removed to create the space needed, a patient not having been exenterated, or eviscerated, will usually require some surgical preparation to create the space needed.

When positioning of the donor heart necessitates the resection of local anatomy to create sufficient room, the chest should be chosen as likely to provide the best position from an overall physiological standpoint. This is because inferior positions impose the need to pump against, rather than with, the force of gravity and to do so over a greater distance, detracting from the total force of ejection of the transplant heart, possibly resulting in the need for inotropes. Also, to pass through the diaphragm requires the surgical preparation of an annulus through which the blood mainlines will pass. Blood mainlines should kept as short as possible and arranged to work with rather than against gravity.

In an inferior position, harder walled mainlines more likely to irritate the surrounding tissue are essential to prevent kinking when the upper body is flexed. Unless some tissue is removed to create sufficient space, encroachment upon neighboring tissue may result in a constant pulsatile sensation which in more irritable patients or when more irritating, may result in the patient insisting that the second heart be removed and an orthotopic transplant performed, or that the added heart be resituated where it is better accommodated. Ideally, a donor heart small enough to allow placement in the chest without the need to remove tissue will be found.

In some cases, such as due to the unavailability of any but a heart larger in size than only the removal of an inadmissible volume of tissue in the chest would allow to be fitted, the use of a position inferior both anatomically and physiologically will be unavoidable. Such is the lower abdomen, addressed below. Meant here for placement in the chests of adults are hearts from smaller adults and older children, not those of infants. This illustrates an advantage in metered switch implanted double heart over conventional heterotopic heart transplantation, in that the implanted automatic response system is able to control the venous return servovalves of the main, that is, the native or earlier transplanted heart, so that each heart drives a volume of blood proportional to its capacity.

In conventional orthotopic heart transplantation, a replacement heart moderately oversized for the recipient gradually adapts to its new milieu by relaxing while one undersized gradually strengthens. But in conventional heterotopic transplantation, a significant size mismatch precipitates failure. Since an advantage of heterotopic transplantation is precisely to allow two less robust hearts to sustain the patient, thus increasing the number of hearts available, the increase in the degree of size mismatch allowed by double heart switch transplantation expands the zone of available hearts all the more.

More specifically, that double heart transplantation makes it possible to compensate for wide discrepancies in the size of a second heart considerably expands the number of hearts that will be available for a specific patient, and most favorably, smaller hearts. These ease the problem of positioning the additional heart in any location, but especially in the chest, where “ . . . right-sided pleuro-pericardial reflection [can be] divided to create space for the donor heart in the right pleural cavity” (Vaijyanath, P. 2017. “A New Technique of Heterotopic Heart Transplantation with Direct Pulmonary Artery Anastomosis,” Journal of the Tehran Heart Center 12(4):194-195).

If necessary and the condition of the patient allows, a partial pneumonectomy with only so much of the right lung as is essential to create sufficient space is removed. The mainlines connecting the native to the donor heart, which need not be hard-walled and more likely to irritate the surrounding tissue, should be routed as close to horizontally as practicable, thus avoiding the greater force of ejection imposed of the donor heart when positioned in an inferior position. Nevertheless, if resection of the lung is contraindicated, then the next best option is to resect tissue in the pelvis to make room.

There is no good location to place a second intact heart; true to the field of medicine in general, almost every remedy other than proper nutrition, exercise, and adequate sleep involves aspects of artificiality that trigger adverse reactions; so that any measure taken must balance the benefits against the known risks. Unlike kidneys and lungs, having provided proper food and circumstances of life necessitating daily exertion, nature provided one heart. One sound heart should be sufficient. Unfortunately, however, dependency upon the mother for oxygenation and nutrition during development liberates the heart to develop unchecked, so that a wide spectrum of anomalies is seen and only postnatally does circulatory insufficiency materialize. This situation is now changing.

The prenatal replacement of the heart too malformed to repair so that normal circulation will result—to include the procedures delineated herein-will avert birth with an unsurvivably malformed heart or any other organ. When not the result of disease such as rheumatic fever, metastatic malignancy, radiation and chemotherapy, uncontrolled hypertension, infection, congenital, or type 1 diabetes, connective tissue disorders, heart disease in those having been born with a normal heart is self-imposed through inactivity, poor nutrition, smoking, and obesity resulting in metabolic syndrome and diabetes. Using measures delineated herein, irreversible heart damage in an adult can be averted through replacement with a sound heart or the addition of a second heart to assist that native with fundamentally more favorable outcomes.

Understanding that the lack of a good location to place a second intact heart is simply given by nature, the order of preference for positioning a second heart from most to least is:
1. In the chest, with the removal of the least volume taken from the bottom of the right lung to provide sufficient space. Currently, lung ‘regeneration’ is limited to the increase in alveoli seen in the remaining lung following a partial or total pneumonectomy, not the redevelopment or reemergence of the lung tissue that had been removed as with removal of a portion of the liver or a bursa. Unless the lungs are also to be replaced, lung disease demands the choice of another location. Upon positioning the donor heart beside that native, direct contact between native and donor hearts is avoided by spacing off the donor from that native, any mobility or ‘wobbling’ of the donor heart which would annoy the recipient by its constant pulsation prevented by filling any objectional voids with a suitable space maintainer such as a balloon. Reference to space fillers such as used in facial plastic surgery contemplate improvements in such products.

If used, suture to tether the donor heart farther from the native heart must hold fast and not cause injury indefinitely. Filler materials carry potential complications better avoided through the use of special purpose means of flap-based stabilization, which might consist of permanent air or water-filled balloons, biocompatible nondegrading sponges, or synthetic mesh degradable or nondegradable slings, or connecting straps. Cosmetic fillers are usually suitable for filling small voids; here where regrowth into the vacated hemithorax is not wanted, bulk materials such as small biocompatible sponges should be tucked about the added heart to stabilize it in position (Brown, L. M., Rannels, S. R., and Rannels, D. E. 2001. “Implications of Post-pneumonectomy Compensatory Lung Growth in Pulmonary Physiology and Disease,” Respiratory Research 2(6):340-347).

Intragastric balloons used in bariatric treatment occasionally migrate; however, used as indicated to fill voids in the chest, migration, as well as several other potential complications, are not possible (see, for example, Lee, P., Tan, W. J., Brown, K. G. M., and Solomon, M. J. 2019. “Addressing the Empty Pelvic Syndrome following Total Pelvic Exenteration: Does Mesh Reconstruction Help?,” Colorectal Disease 21(3):365-369; Mukherjee, S., Darzi, S., Paul, K., Werkmeister, J. A., and Gargett, C. E. 2019. “Mesenchymal Stem Cell-based Bioengineered Constructs: Foreign Body Response, Cross-talk with Macrophages and Impact of Biomaterial Design Strategies for Pelvic Floor Disorders,” Interface Focus 9(4):20180089; Turkeltaub, J. A. and Edmundowicz, S. A. 2019. “Endoscopic Bariatric Therapies: Intragastric Balloons, Tissue Apposition, and Aspiration Therapy,” Current Treatment Options in Gastroenterology 17(2):187-201; Choi, S. J. and Choi, H. S. 2018. “Various Intragastric Balloons under Clinical Investigation,” Clinical Endoscopy 51(5):407-415; Laing, P., Pham, T., Taylor, L. J., and Fang, J. 2017. “Filling the Void: A Review of Intragastric Balloons for Obesity,” Digestive Diseases and Sciences 62(6):1399-1408; Schiltz, B., Buchs, N. C., Penna, M., Scarpa, C. R., Liot, E., Morel, P., and Ris, F. 2017. “Biological Mesh Reconstruction of the Pelvic Floor following Abdominoperineal Excision for Cancer: A Review,” World Journal of Clinical Oncology 8(3):249-254; Lo, T. S., Darzi, S., Urbankova, I., Su, K., White, J., Lo, C., and 4 others 2016. “Tissue Response to Collagen Containing Polypropylene Meshes in an Ovine Vaginal Repair Model,” Acta Biomaterialia 2016 39:114-123; Lin, Y. H., Yusoff, F. M., Chu, H. C., Hsieh, W. C., and Uy-Patrimonio, M. C. 2016. “The Immunohistochemical and Urodynamic Evaluation towards the Collagen-coated and Non-coated Polypropylene Meshes Implanted in the Pelvic Wall of the Rats,” (Nature Research) Scientific Reports 6:38960; Zhang, Q., Lin, S. R., He, F., Kang, D. H., Chen, G. Z., and Luo, W. 2011. “Water-filled Balloon in the Postoperative Resection Cavity Improves Dose Distribution to Target Volumes in Radiotherapy of Maxillary Sinus Carcinoma,” Chinese Journal of Cancer 30(11):786-793).

A filler or space retainer of any kind should be used if and only if it is clear that without such use, the patient would experience discomfort to which the patient would not become acclimated (see, for example, Herold, J. and Kalucka, J. 2021. “Angiogenesis in Adipose Tissue: The Interplay between Adipose and Endothelial Cells,” Online, Frontiers in Physiology (Lausanne, Switzerland) 11:624903; Abu-Ghname, A., Perdanasari, A. T., and Reece, E. M. 2019. “Principles and Applications of Fat Grafting in Plastic Surgery,” Seminars in Plastic Surgery 33(3):147-154; Doornaert, M., Colle, J., De Maere, E., Declercq, H., and Blondeel, P. 2019. “Autologous Fat Grafting: Latest Insights,” Annals of Medicine and Surgery (London, England) 37:47-53; Fontes, T., Brandão, I., Negrão, R., Martins, M. J., and Monteiro, R. 2018. “Autologous Fat Grafting: Harvesting Techniques,” Annals of Medicine and Surgery (London, England) 36:212-218; Bellini, E., Grieco, M. P., and Raposio, E. 2017. “The Science behind Autologous Fat Grafting,” Annals of Medicine and Surgery (London, England) 24:65-73; Simonacci, F., Bertozzi, N., Grieco, M. P., Grignaffini, E., and Raposio, E. 2017. “Procedure, Applications, and Outcomes of Autologous Fat Grafting,” Annals of Medicine and Surgery (London, England) 20:49-60; Banyard, D. A., Borad, V., Amezcua, E., Wirth, G. A., Evans, G. R., and Widgerow, A. D. 2016. “Preparation, Characterization, and Clinical Implications of Human Decellularized Adipose Tissue Extracellular Matrix (hDAM): A Comprehensive Review,” Aesthetic Surgery Journal 36(3):349-357; Zielins, E. R., Brett, E. A., Longaker, M. T., and Wan, D. C. 2016. “Autologous Fat Grafting: The Science behind the Surgery,” Aesthetic Surgery Journal 36(4):488-496; Suszynski, T. M., Sieber, D. A., Van Beek, A. L., and Cunningham, B. L. 2015. “Characterization of Adipose Tissue for Autologous Fat Grafting,” Aesthetic Surgery Journal 35(2):194-203; Dolderer, J. H., Medved, F., Haas, R. M., Siegel-Axel, D. I., Schiller, S. M., and Schaller, H. E. 2013. “Angiogenesis and Vascularisation in Adipose Tissue Engineering,” (in German with English abstract at Pubmed), Handchirurgie, Mikrochirurgie, Plastische Chirurgie 45(2):99-107).

Fat implantation with platelet rich plasma has been found to also promote healing (Smith, O. J., Jell, G., and Mosahebi, A. 2019. “The Use of Fat Grafting and Platelet-rich Plasma for Wound Healing: A Review of the Current Evidence,” International Wound Journal 16(1):275-285; Yamakawa, S. and Hayashida, K. 2019. “Advances in Surgical Applications of Growth Factors for Wound Healing,” Burns and Trauma 7:10; Smith, O. J., Kanapathy, M., Khajuria, A., Prokopenko, M., Hachach-Haram, N., Mann, H., and Mosahebi, A. 2018. “Systematic Review of the Efficacy of Fat Grafting and Platelet-rich Plasma for Wound Healing,” International Wound Journal 15(4):519-526).

The introduction of a filler about the donor heart must be performed in an open field with a clear view of the vasculature. While associated with facial cosmetic procedures where the vasculature underlying the point of injection was not visible and migration from the point of injection appears not to have been a factor, the accidental introduction of a filler into a vessel can lead to vascular problems, pain due to anoxia, and at least in the face, even embolism-induced blindness (Chatrath, V I., Banerjee, P. S., Goodman, G. J., and Rahman, E. 2019. “Soft-tissue Filler-associated Blindness: A Systematic Review of Case Reports and Case Series,” Plastic and Reconstructive Surgery. Global Open 7(4):e2173; Rullan, P. P. and Lee, K. C. 2019. “Successful Management of Extreme Pain from Delayed Embolization after Hyaluronic Acid Filler Injection,” Journal of the American Academy of Dermatology Case Reports 5(7):569-571; Sito, G., Manzoni, V., and Sommariva, R. 2019. “Vascular Complications after Facial Filler Injection: A Literature Review and Meta-analysis,” Journal of Clinical and Aesthetic Dermatology 12(6):E65-E72; de Lacerda, D. 2018. “Prevention and Management of latrogenic Blindness Associated with Aesthetical Filler Injections,” Dermatologic Therapy 31(6):e12722). Nevertheless, such consequences in the chest under clear view exist as remote possibilities such that only staff with experience in the use of fillers and reversal agents such as hyaluronidase to reverse complications as might arise from their use should work with these materials.

Autologous or autogenic fat avoids complicating immunological reactions best avoided at transplantation but tends to shrink, a property restrainable through use of a one-to-one mix of fat and hyaluronan hydrogel (Alghoul, M., Mendiola, A., Seth, R., Rubin, B. P., Zins, J. E., and 3 others 2012. “The Effect of Hyaluronan Hydrogel on Fat Graft Survival,” Aesthetic Surgery Journal 32(5):622-633) and the percutaneous administration of dimethylsulfoxide (Sari, E., Bakar, B., Sarkarati, B., Bozdogan, O., and Cavusoglu, T. 2016. “Effectiveness of Dimethyl sulfoxide on the Survival and Volume Preservation of Autologous Fat Graft Tissue: A Preliminary Study,” Aesthetic Surgery Journal 36(2):NP58-67) and/or fructose 1,6-Bisphosphate (Lv, T., Gu, Y., Bi, J., Kang, N., Yang, Z., and 6 others 2019. “Fructose 1,6-bisphosphate as a Protective Agent for Experimental Fat Grafting,” Stem Cells Translational Medicine 8(6):606-616).

If necessary, once the surgery has healed, the filler can be replaced with a hydrogel/fat graft with little difficulty (see, for example, Bhat, D., Kachniarz, B., Seu, M., Li, X, Martin, R., and 4 others 2019. “Abstract 71: Improved Fat Graft Retention with Nanofiber-Hydrogel Tissue Scaffold in an in-Vivo Murine Model,” Plastic and Reconstructive Surgery. Global Open April 29 7(4 Supplement): 50-51; Bhat, D., Kachniarz, B., Seu, M., Li, X., Martin, R., and 4 others 2018. “Abstract: A Novel Nanofiber-Hydrogel Tissue Scaffold for Improved Fat Graft Survival,” Plastic and Reconstructive Surgery. Global Open 69 Supplement): 150-152; Lui, Y. F. and Ip, W. Y. 2016. “Application of Hydrogel in Reconstruction Surgery: Hydrogel/Fat Graft Complex Filler for Volume Reconstruction in Critical Sized Muscle Defects,” Online, BioMed Research International 2016:3459431).

Many other fillers, notably, silicon, are to be avoided (see, for example, Haneke, E. 2019. “Adverse Effects of Fillers,” Dermatologic Therapy 32(2):e12676; Facial Plastic Surgery 35(3):224-229; Liu, M. H., Beynet, D. P., and Gharavi, N. M. 2019. “Overview of Deep Dermal Fillers,” Facial Plastic Surgery 35(3):224-229; Haneke, E. 2015. “Managing Complications of Fillers: Rare and Not-So-Rare,” Journal of Cutaneous and Aesthetic Surgery 8(4):198-210; Ahn, C. S. and Rao, B. K. 2014. “The Life Cycles and Biological End Pathways of Dermal Fillers,” Journal of Cosmetic Dermatology 13(3):212-223).

Unless the second heart is atypically small, the chest will bulge somewhat to result in a gap in the ribs. The bulge will seldom pose a significant cosmetic problem, but the transected ends of the ribs separated, will expose the heart to the risk of injury. It is therefore imperative that the ribcage be returned to its former rigidity. Protection of the second heart by reinstatement of a solid cage can be achieved with intramedullary rods, or ‘nails,’ bone autografts, tissue engineered bone, bone substitutes, or a combination of these. Unlike a fracture of a long bone such as the tibia where the facing ends of the broken bones return to a flush or substantially flush fit, when a short solid stainless steel or titanium rod is used to reconnect the ribs, the rod will span across a gap.

It being imperative that the new heart not be placed under compression, or tamponade, even if the transected opposing ends of the ribs approximate, the operator should not attempt to reduce the breach into juxtaposed relation, which would afford no protection for two months and weeks longer for adequate protection in any event. A preferred method for allowing the ribs to knit affords protection immediately after the transplant. This consists of percutaneous entry through a small incision aligned to the rib at the flank front to allow insertion of first, a guidewire, then a flexible reamer to bore a channel through the medullary cavity.

A stainless steel or titanium rod of like curvature as the rib, having a small proximal eyelet to allow its later retraction if prospective as in a child, is inserted into the medullary cavity to bridge across the gap. In the elderly, the rods are allowed to remain. Due to the curvature of the ribs, it is not necessary to insert orthopedic angle markers into each rib to be transected prior to thoracotomy as would be needed in lengthening a long bone in a limb, for example. The length of time the rods are allowed to remain depends upon the length of the gaps. Due to the critical need to protect the heart, when the gap or cleft created is small, the rods should be left in for six months per centimeter of the gap to allow bone fill-in.

For larger gaps, once the rods have been placed, small incisions are made overlying the rods exposed in the gaps. Numerous methods and materials are available to fill in the gaps, those acceptable permitting vascularization preclusive of osteonecrosis, or avascular necrosis, which would weaken the rib once the rod was withdrawn; however, consideration should be given to withdrawal of the rods only in a small child, where growth would proportionally reduce the size of the rods, and therewith, the protection these provide. Due to the irregular shape of the bone to be bridged, harvesting autogenous bone from the iliac crest, for example, poses difficulty to reshape into the segment of a rib.

However, if preferred, bone harvested thus or tissue engineered in situ can be sectioned for insertion into the gaps as allogeneic bone chips, for example, and bound into the off-annular shape required with bone cement before insertion of the guidewire and flexible reamer (see, for example, Diaz-Rodriguez, P., Lopez-Alvarez, M., Serra, J., González, P., and Landin, M. 2019. “Current Stage of Marine Ceramic Grafts for 3D Bone Tissue Regeneration,” Marine Drugs 17(8). pii: E471; Du, Y., Guo, J. L., Wang, J., Mikos, A. G., and Zhang, S. 2019. “Hierarchically Designed Bone Scaffolds: From Internal Cues to External Stimuli,” Biomaterials 218:119334; Freitas, J., Santos, S. G., Gonçalves, R. M., Teixeira, J. H., Barbosa, M. A., and Almeida, M. I. 2019. “Genetically Engineered-MSC [mesenchymal stem/stromal cell] Therapies for Non-unions, Delayed Unions, and Critical-size Bone Defects,” Online, MDPI [Multidisciplinary Digital Publishing Institute] International Journal of Molecular Sciences (Basel, Switzerland) 20(14). pii: E3430; Gomez-Barrena, E., Rosset, P., Gebhard, F., Hernigou, P., Baldini, N., and 27 others 2019. “Feasibility and Safety of Treating Non-unions in Tibia, Femur and Humerus with Autologous, Expanded, Bone Marrow-derived Mesenchymal Stromal Cells Associated with Biphasic Calcium Phosphate Biomaterials in a Multicentric, Non-comparative Trial,” Biomaterials 196:100-108; Gupta, S., Malhotra, A., Jindal, R., Garg, S. K., Kansay, R., and Mittal, N. 2019. “Role of Beta Tri-calcium Phosphate-based Composite Ceramic as Bone-graft Expander in Masquelet's-induced Membrane Technique,” Indian Journal of Orthopaedics 53(1):63-69; Karadjian, M., Essers, C., Tsitlakidis, S., Reible, B., Moghaddam, A., Boccaccini, A. R., and Westhauser, F. 2019. “Biological Properties of Calcium Phosphate Bioactive Glass Composite Bone Substitutes: Current Experimental Evidence,” Online, MDPI [Multidisciplinary Digital Publishing Institute] International Journal of Molecular Sciences (Basel, Switzerland) 20(2). pii: E305; Lu, J., Wang, Q. Y., and Sheng, J. G. 2019. “Exosomes in the Repair of Bone Defects: Next-generation Therapeutic Tools for the Treatment of Nonunion,” Online, BioMed Research International 2019:1983131; Fernandez de Grado, G., Keller, L., Idoux-Gillet. Y., Wagner, Q., Musset, A. M., and 3 others 2018. “Bone Substitutes: A Review of Their Characteristics, Clinical Use, and Perspectives for Large Bone Defects Management,” Journal of Tissue Engineering 9:2041731418776819; Sassoli, C., Vallone, L., Tani, A., Chellini, F., Nosi. D., and Zecchi-Orlandini, S. 2018. “Combined Use of Bone Marrow-derived Mesenchymal Stromal Cells (BM-MSCs) and Platelet Rich Plasma (PRP) Stimulates Proliferation and Differentiation of Myoblasts in Vitro: New Therapeutic Perspectives for Skeletal Muscle Repair/Regeneration,” Cell and Tissue Research 372(3):549-570; Offner, D.3. Wagner, Q., Idoux-Gillet. Y., Gegout, H., Ferrandon, A., and 4 others 2017. “Hybrid Collagen Sponge and Stem Cells as a New Combined Scaffold Able to Induce the Re-organization of Endothelial Cells into Clustered Networks,” Biomedical Materials and Engineering 28 (s1):S185-S192; Shanbhag, S., Pandis, N., Mustafa, K., Nyengaard, J. R., and Stavropoulos, A. 2017. “Cell Cotransplantation Strategies for Vascularized Craniofacial Bone Tissue Engineering: A Systematic Review and Meta-analysis of Preclinical in Vivo Studies,” Tissue Engineering. Part B. Reviews 23(2):101-117; Shanbhag. S., Pandis, N., Mustafa, K., Nyengaard, J. R., and Stavropoulos, A. 2017. “Alveolar Bone Tissue Engineering in Critical-size Defects of Experimental Animal Models: A Systematic Review and Meta-analysis,” Journal of Tissue Engineering and Regenerative Medicine 11(10):2935-2949; Malhotra, A. and Habibovic. P. 2016. “Calcium Phosphates and Angiogenesis: Implications and Advances for Bone Regeneration,” Trends in Biotechnology 34(12):983-992; Wagner, Q., Offner, D., Idoux-Gillet, Y., Saleem, I., Somavarapu, S., and 3 others 2016. “Advanced Nanostructured Medical Device Combining Mesenchymal Cells and VEGF [vascular endothelial growth factor] Nanoparticles for Enhanced Engineered Tissue Vascularization,” Nanomedicine (London, England) 11(18):2419-2430; Geiger, F., Beverungen, M., Lorenz, H., Wieland, J., Fehr, M., and Kasten, P. 2012. “Bone Substitute Effect on Vascularization and Bone Remodeling after Application of ph VEGF165 [vascular endothelial growth factor] Transfected BMSC [bone marrow stromal cells],” Journal of Functional Biomaterials 3(2):313-326; Schmidt-Bleek, K., Schell, H., Schulz, N., Hoff, P., Perka, C., and 4 others 2012. “Inflammatory Phase of Bone Healing Initiates the Regenerative Healing Cascade,” Cell and Tissue Research 347(3):567-573; Giannoudis, P. V., Einhom, T. A., Schmidmaier, G., and Marsh, D. 2008. “The Diamond Concept-Open Questions,” Injury 39 Supplement 2: S5-S8; Saikia, K. C., Bhattacharya, T. D., Bhuyan, S. K., Talukdar, D. J., Saikia. S. P., and Jitesh, P. 2008. “Calcium Phosphate Ceramics as Bone Graft Substitutes in Filling Bone Tumor Defects,” Indian Journal of Orthopaedics 42(2):169-172; Galois, L. and Mainard, D. 2004. “Bone Ingrowth into two Porous Ceramics with Different Pore Sizes: An Experimental Study,” Acta Orthopaedica Belgica 70(6):598-603; Głowacki, J. 1998. “Angiogenesis in Fracture Repair,” Clinical Orthopedics and Related Research(355 Supplement): S82-S89).

In a small child, unless to harvest a skin allograft poses no special problem, a second heart should be selected for minimal forward protrusion and stretching of the skin without extended tissue expander application to span the surgical defect in the rib or ribs of greatest anterior displacement. In the elderly, the skin will have lost much resistance to stretching and the cosmetic factor will usually be less important to the patient, affording greater latitude in the size of the second heart.

The process of alveolar regeneration following partial pneumonectomy is expedited when the remaining lung undergoes stress as from exercise, but will be sufficiently progressed to achieve full or nearly full compensation gradually over a period of years following the procedure (Butler, J. P., Loring, S. H., Patz, S., Tsuda, A., Yablonskiy, D. A., and Mentzer, S. J. 2012. “Evidence for Adult Lung Growth in Humans,” New England Journal of Medicine 367(3):244-247; additional references provided below in this section). Provided both lungs are intact, a partial right pulmonectomy has far less potential to provoke adverse sequelae than does a partial hepatectomy as addressed below in this section.

More specifically, the amount of the lower right lung removed ordinarily includes the inferior lobe and about half of the lower middle lobe. Given that following a partial pulmonectomy, the remaining lung internally regenerates as delineated below in this section, the ‘tradeoff’ involved in losing this volume of lung tissue in order to overcome the circulatory insufficiency of a congenitally mal formed or severely decompensated heart in failure and the prospects these conditions presage is especially favorable. Once the process of regeneration has progressed, the exercise or exertion capacity of the patient will considerably exceed that preoperative and will continue to increase as the lung regenerative capacity for recovery continues.

Indeed, few patients will have had much if any preoperative capacity for exercise, which undertaken, then fundamentally improves health. That in the chest, the hearts are adjacent rather than widely separated necessitates the use of implantable rather than wearable cardioverter defibrillators when the need for these is evident. Nevertheless, on balance, this is the best position for the second heart. The costly implantable devices are recoverable with little if any trauma while the transplantation should last for many years.

In marked contrast to a conventional heterotopic heart transplant where atrial merger of the native with a smaller second heart presages a poor outcome if not failure, in a double heart transplant, use of a smaller second intact heart is actually preferred as facilitating relatively uncomplicated placement in the chest with the least removal of lower right lung tissue during the lower right pulmonectomy needed to fit it, and once placed, will be subjected to a lesser load than were it positioned in one of the anatomically as well as physiologically inferior locations delineated below. Placement in the lower abdomen, for example, is best limited to the eventuality that only a larger heart is available.

Where the extent to which the donor heart can be smaller is limited due to the difference in dimensions of the atria to be merged in the conventional procedure, in a double heart transplant, no merger and therefore no such limitation applies. Such a heart is better equipped to deal with the increase in load posed by longer blood-conveying mainlines to reach upwards (cephalad, craniad, superiorly) to the native or earlier-transplanted heart in the chest. To an extent, the increased force of ejection requiring a larger ventricular absolute stroke volume and stronger ventricular myocardial walls, which positioned in the chest would induce pulmonary hypertension resulting in coma and convulsions pending adaptation of the heart to its new circumstances is avoided.

Furthermore, unlike the conventional method, in a double heart transplant, the native and donor hearts remain physically separate and therefore segregably addressable for diagnostic and therapeutic treatment by the implant disorder response system, using means described in U.S. Pat. No. 11,759,186. The release of supportive medication by direct pipeline targeting executed by the implant disorder control system microcontroller serves to avert any adverse postimplantation sequelae, such as pulmonary hypertension due to the additive increase in the pressure of the combined ventricular ejection fractions, or absolute stroke volumes. Here the recipient and donor hearts are in a state of failure, making such an eventuality improbable.

Dispelled thus are the risks for coma and convulsion that follow the conventional orthotopic implantation of an oversized heart until it has had time to adapt to the reduced burden placed upon it. While not pertinent to the volume and shape of lung tissue removed, ‘true,’ that is, endogenous, spontaneous, regeneration of pulmonectomized tissue does occur, the regeneration thereof consisting rather of an increase in the density of alveoli. Provided the volume of tissue removed is kept to a minimum, this allows the eventual recovery to relatively unimpaired gas exchange.

As have attempts at devising implantable artificial lungs, to date, the application of exogenous stem cells to prompt lung regeneration has met with little success (see, for example, Basil, M. C. and Morrisey, E. E 2019. “BASC-ing [bronchioalveolar stem cells] in the Glow: Bronchioalveolar Stem Cells Get Their Place in the Lung,” European Molecular Biology Organization Journal 38(12). pii: e102344; Finn, J., Sottoriva, K., Pajcini, K. V., Kitajewski, J. K., Chen, C., and 3 others 2019. “DIk1-mediated Temporal Regulation of Notch Signaling is Required for Differentiation of Alveolar Type II to Type I Cells during Repair,” Cell Reports (Cambridge, Massachusetts) 26(11):2942-2954.e5; Hu. C., Sun, J., Du, J.1, Wen, D., Lu, H., and 6 others 2019. “The Hippo-YAP [protein kinase Hpo YES (proto-oncogene tyrosine-protein kinase Yes)-associated protein] Pathway Regulates the Proliferation of Alveolar Epithelial Progenitors after Acute Lung Injury,” Cell Biology International January 11; Katsura, H., Kobayashi, Y., Tata, P. R., and Hogan, B. L. M. 2019. “IL-1 and TNFα Contribute to the Inflammatory Niche to Enhance Alveolar Regeneration,” Stem Cell Reports 12(4):657-666; LaCanna. R., Liccardo, D., Zhang, P., Tragesser, L., Wang. Y., and 8 others 2019. “Yap/Taz [transcriptional regulators/effectors of the Hippo pathway/Yes- associated protein/transcriptional co-activator with PDZ-binding motif] Regulate Alveolar Regeneration and Resolution of Lung Inflammation,” Journal of Clinical Investigation 129(5):2107-2122; Li, C., Lee, M. K., Gao, F., Webster, S., Di. H., and 9 others 2019. “PDGFRalpha [platelet-derived growth factor receptor alpha] Controls the Elastogenesis Pathway via a Secondary Tier of Signaling Networks during Alveologenesis,” Online, Development 146(15):dev176354; Liu, Q., Liu, K., Cui, G., Huang, X., Yao. S., and 22 others 2019. “Lung Regeneration by Multipotent Stem Cells Residing at the Bronchioalveolar-Duct Junction,” Nature Genetics 51(4):728-738; Mammoto, T., Muyleart. M., and Mammoto, A. 2019. “Endothelial YAPI in Regenerative Lung Growth through the Angiopoietin-Tie2 Pathway,” American Journal of Respiratory Cell and Molecular Biology 60(1):117-127; McQualter, J. L. 2019. “Endogenous Lung Stem Cells for Lung Regeneration,” Expert Opinion on Biological Therapy 19(6):539-546; Moya, I. M. and Halder, G. 2019. “Hippo-YAP/TAZ Signalling in Organ Regeneration and Regenerative Medicine,” Nature Reviews. Molecular Cell Biology 20(4):211-226; Olajuyin, A. M., Zhang, X., and Ji. H. L. 2019. “Alveolar Type 2 Progenitor Cells for Lung Injury Repair,” Cell Death Discovery 5:63; Salwig. I., Spitznagel, B., Vazquez-Armendariz, A. I., Khalooghi, K., Guenther, S., and 3 others 2019. “Bronchioalveolar Stem Cells are a Main Source for Regeneration of Distal Lung Epithelia in Vivo,” European Molecular Biology Organization Journal 38(12). pii: e102099; Sun, T., Huang, Z., Zhang, H., Posner, C., Jia, G., and 6 others 2019. “TAZ [WW, rsp5-domain. WWP repeating motif domain- containing transcription regulator protein 1] is Required for Lung Alveolar Epithelial Cell Differentiation after Injury,” Journal of Clinical Investigation Insight 5. pii: 128674; Swarr, D. T., Herriges, M., Li, S., Morley, M., Fernandes, S., and 5 others 2019. “The Long Noncoding RNA Falcor Regulates Foxa2 Expression to Maintain Lung Epithelial Homeostasis and Promote Regeneration,” Genes and Development 33(11-12):656-668; Whitsett, J. A., Kalin, T. V., Xu. Y., and Kalinichenko, V. V. 2019. “Building and Regenerating the Lung Cell by Cell,” Online, Physiological Reviews 99(1):513-554; Zepp, J. A. and Morrisey, E. E. 2019. Cellular Crosstalk in the Development and Regeneration of the Respiratory System, Online, Nature Reviews. Molecular Cell Biology 20(9):551-566; Cao. X. Y. and Xiao, S. Y. 2018. “Chronic Lung Disease, Lung Regeneration and Future Therapeutic Strategies,” Chronic Disease and Translational Medicine 4(2):103-108; Chung, M. I., Bujnis, M., Barkauskas, C. E., Kobayashi, Y., and Hogan, B. L. M. 2018. “Niche-mediated BMP/SMAD [bone morphogenetic protein/small worm and Drosophila gene] Signaling Regulates Lung Alveolar Stem Cell Proliferation and Differentiation,” Development (Cambridge, England) 145(9). pii: dev163014; Gouveia, L., Betsholtz, C., and Andrae, J. 2018. “PDGF-A [platelet-derived growth factor A] Signaling is Required for Secondary Alveolar Septation and Controls Epithelial Proliferation in the Developing Lung,” Online, Development 145(7):ev161976; Xie. H., Wu, L., Deng. Z., Huo, Y., and Cheng, Y. 2018. “Emerging Roles of YAP/TAZ in Lung Physiology and Diseases,” Life Sciences 214:176-183; Zhu, Y., Chen, X., Yang, X., and El-Hashash, A. 2018. “Stem Cells in Lung Repair and Regeneration: Current Applications and Future Promise,” Journal of Cellidar Physiology 233(10):6414-6424; Bolte, C., Flood, H. M., Ren, X., Jagannathan, S., Barski, A., Kalin, T. V., and Kalinichenko, V. V. 2017. “FOXF1 Transcription Factor Promotes Lung Regeneration after Partial Pneumonectomy,” (Nature Research) Scientific Reports 7(1):10690; Fu. S. L., Zhao, W. Y., Zhang, W. J.3. Song. H., Ji. H. B., and Tang, N. 2017. “Hippo Signaling Pathway in Lung Development, Regeneration, and Diseases,” (in Chinese with English abstract at Pubmed), Yi Chuan [Hereditas] 39(7):597-606; Xi. Y., Kim. T., Brumwell, A. N., Driver, I. H., Wei. Y., and 9 others 2017. “Local Lung Hypoxia Determines Epithelial Fate Decisions during Alveolar Regeneration,” Nature Cell Biology 19(8):904-914; Akram, K. M., Patel, N., Spiteri, M. A., and Forsyth, N. R. 2016. “Lung Regeneration: Endogenous and Exogenous Stem Cell Mediated Therapeutic Approaches,” Online, MDPI [Multidisciplinary Digital Publishing Institute] International Journal of Molecular Sciences (Basel, Switzerland) 17(1). pii: E128; Chao, C.-M., Moiseenko, A., Zimmer, K.-P., and Bellusci, S. 2016. “Alveologenesis: Key Cellular Players and Fibroblast Growth Factor 10 Signaling,” Online, Molecular and Cellular Pediatrics 3(1):17; Gandhirajan, R. K., Jain, M., Walla, B., Johnsen, M., Bartram, M. P., and 4 others 2016. “Cysteine S-Glutathionylation Promotes Stability and Activation of the Hippo Downstream Effector Transcriptional Co-activator with PDZ-binding Motif (TAZ),” Journal of Biological Chemistry 291(22):11596-11607; Juan, W. C. and Hong. W. 2016. “Targeting the Hippo Signaling Pathway for Tissue Regeneration and Cancer Therapy,” Genes (Basel, Switzerland) 7(9). pii: E55; Schilders, K. A., Eenjes, E., van Riet, S., Poot, A. A., Stamatialis, D., and 3 others 2016. “Regeneration of the Lung: Lung Stem Cells and the Development of Lung Mimicking Devices,” Respiratory Research 17:44; Lange, A. W., Sridharan, A., Xu. Y., Stripp. B. R., Perl, A. K., and Whitsett, J. A. 2015. “Hippo/Yap [cell proliferation, organ size, regeneration, cell differentiation, stemness, survival, apoptosis, and tumorigenesis component of the hippo signaling pathway] Signaling Controls Epithelial Progenitor Cell Proliferation and Differentiation in the Embryonic and Adult Lung,” Online, Journal of Molecular Cell Biology 7(1):35-47; Herriges, M. and Morrisey, E. E. 2014. “Lung Development: Orchestrating the Generation and Regeneration of a Complex Organ,” Development (Cambridge, England) 141(3):502-513; Hogan, B. L., Barkauskas, C. E., Chapman, H. A., Epstein, J. A., Jain, R., and 13 others 2014. “Repair and Regeneration of the Respiratory System: Complexity, Plasticity, and Mechanisms of Lung Stem Cell Function,” Cell Stem Cell 15(2):123-138; Kotton, D. N. and Morrisey, E. E. 2014. “Lung Regeneration: Mechanisms, Applications and Emerging Stem Cell Populations,” Nature Medicine 20(8):822-832. (also Op cit. above in section 2a; entitled Background. General: Thane. K., Ingenito, E. P., and Hoffman, A. M. 2014. “Lung Regeneration and Translational Implications of the Postpneumonectomy Model,” Translational Research 163(4):363-376; Weiss, D. J. 2014. “Concise Review: Current Status of Stem Cells and Regenerative Medicine in Lung Biology and Diseases,” Stem Cell 32(1):16-25; Wansleeben, C., Barkauskas, C. E., Rock, J. R., and Hogan, B. L. 2013. “Stem Cells of the Adult Lung: Their Development and Role in Homeostasis, Regeneration, and Disease,” Wiley Interdisciplinary Reviews Developmental Biology 2(1):131-148; Becker, P. M., Tran, T. S., Delannoy, M. J., He, C., Shannon, J. M., and McGrath-Morrow, S. 2011. “Semaphorin 3A [neuronal developmental agent involved in cellular proliferation, migration, and apoptosis] Contributes to Distal Pulmonary Epithelial Cell Differentiation and Lung Morphogenesis,” Online, PLoS [public library of science] One 6(11):e27449; Kubo, H. 2011. “Molecular Basis of Lung Tissue Regeneration,” General Thoracic and Cardiovascular Surgery 59(4):231-244; Bertoncello, I. and McQaulter, J. L. 2010. Endogenous Lung Stem Cells: What is Their Potential for Use in Regenerative Medicine?,” Expert Review of Respiratory Medicine 4(3):349-362; Weiss, D. J., Kolls, J. K., Ortiz, L. A., Panoskaltsis-Mortari. A., and Prockop, D. J. 2008. “Stem Cells and Cell Therapies in Lung Biology and Lung Diseases,” Online, Proceedings of the American Thoracic Society 5(5):637-667; Zhang. Y., Goss, A. M., Cohen, E. D., Kadzik, R., Lepore, J. J., and 6 others 2008. “A Gata6-Wnt ([Wingless and Int-1] transduction pathways that gain entry into the cell through its surface receptors) Pathway Required for Epithelial Stem Cell Development and Airway Regeneration,” Nature Genetics 40(7):862-870; Kannan, S. and Wu, M. 2006. “Respiratory Stem Cells and Progenitors: Overview, Derivation, Differentiation, Carcinogenesis, Regeneration, and Therapeutic Application,” Current Stem Cell Research and Therapy 1(1):37-46; Neuringer, I. P. and Randell. S. H. 2004. “Stem Cells and Repair of Lung Injuries,” Online, BioMed Central Respiratory Research 5(1):6).

2. Infrarenally in the lower right or left quadrant of the abdomen. To create sufficient space to place a second heart within a protective cage described below in this section, a length of gut much too short to result in short bowel syndrome is excised. The wearing of an armor stomach-plankart while engaged in an activity such as driving a motor vehicle is a poor substitute for an implanted heart cage as described below in section 2b(1)(b)(b6), entitled Heart Cage for the Infrarenal Abdominal Positioning of a Second Heart, which will afford protection against injury that could not have been anticipated. Due to the resistance posed by the longer blood-conveying mainlines and the force of gravity, position thus is inferior to and suitable only when placement in the chest is contraindicated. Foreign body reactions and the formation of adhesions are addressed above in section 2a, entitled Background. General.

3. In the pelvis. In a patient who had undergone a pelvic evisceration, or exenteration, but has since been confirmed to be malignancy free, the evacuated pelvis affords bony protection and eliminates the need for a midsternal or a clamshell thoracotomy. Otherwise, the removal of gastrointestinal or urogenital structure for the purpose of securing a functionally less advantageous position to place a second heart is discouraged. Placed thus, the left ventricle, likely impaired in a marginal donor heart, must drive the oxygenated blood cephalad against gravity through a longer aortic mainline. Exceptionally, a larger heart with stronger absolute stroke volume can be placed thus with the excessive force of ejection restrained. Otherwise, positioning thus is inferior even to placement infrarenally within the abdomen.

4. In the upper abdomen. Positioning thus is acceptable only when a younger patient is morbidly, Class II (body mass index over 35) or Class III (BMI over 40) obese, so that stomach reduction surgery would be beneficial in any event. The lower ribs afford a measure of protection; however, compared to placement in the chest, where the hearts are adjacent with blood-conveying mainlines, or bloodlines, substantially horizontal and relatively short, the bloodlines with placement in the upper abdomen are relatively long and the left ventricle sees the added load of gravity. If it must be resected, the liver will regenerate to recreate the normal mass within the available space but not necessarily its former shape.

In a pediatric patient, regeneration to the original mass if not shape following a ⅔ partial hepatectomy takes at least an entire year. In a less developed part of the world, a position thus can serve as an organic rather than a costly mechanical assist device which also allows avoiding a midsternal or clamshell thoracotomy. Otherwise, placement thus is warranted when severe thoracic injury or infection interferes with placement in the chest. To sustain the heart in the upper abdomen may necessitate periodic endoscopic debulking or partial hepatectomy of the liver tissue regenerated over the preceding year or year and a half. Repetitive reduction of the liver so that the patient is repeatedly left hepatically impaired would likely result in hepatocardiac syndrome and the need for liver dialysis.

If blocked from growth, regeneration without periodic endoscopic reduction could result in protrusion of the liver into the body wall, creating discomfort and increased susceptibility to injury, as well as a cosmetic problem (Fausto, N. 2000. “Liver Regeneration,” Journal of Hepatology 32(1 Supplement): 19-31)(see also, for example, Abu Rmilah, A., Zhou, W., Nelson, E., Lin, L., Amiot, B., and Nyberg, S. L. 2019. Understanding the Marvels behind Liver Regeneration,” Wiley Interdisciplinary Reviews. Developmental Biology 8(3):e340; Chen, Y., Hata. T., Rehman, F., Kang, L., Yang. L., Kim, B. Y. S., and Nguyen, J. H. 2019. “Visualization of Hepatocellular Regeneration in Mice after Partial Hepatectomy,” Journal of Surgical Research 235:494-500; Gilgenkrantz, H. and Collin de l'Hortet, A. 2018. “Understanding Liver Regeneration: From Mechanisms to Regenerative Medicine,” American Journal of Pathology 188(6):1316-1327; Zhang, J., Zhao, X., Liang, L., Li, J., Demirci, U., and Wang, S. 2018. “A Decade of Progress in Liver Regenerative Medicine,” Biomaterials 157:161-176; Schwabe, R. F. and Luedde. T. 2018. “Apoptosis and necroptosis in the Liver: A Matter of Life and Death,” Nature Reviews. Gastroenterology and Hepatology 15(12):738-752; Zhou, H., Xie. G., Mao, Y., Zhou, K., Ren, R., and 3 others 2018. “Enhanced Regeneration and Hepatoprotective Effects of Interleukin 22 Fusion Protein on a Predamaged Liver Undergoing Partial Hepatectomy,” Journal of Immunology Research 2018:5241526; Kholodenko, I. V. and Yarygin, K. N. 2017. “Cellular Mechanisms of Liver Regeneration and Cell-based Therapies of Liver Diseases,” Online, BioMed Research International 2017:8910821; Nicolas, C. T., Hickey, R. D., Chen, H. S., Mao, S. A., Lopera Higuita, M., Wang. Y., and Nyberg, S. L. 2017. “Concise Review: Liver Regenerative Medicine: From Hepatocyte Transplantation to Bioartificial Livers and Bioengineered Grafts” Stem Cells 35(1):42-50; Hu. M., Li, S., Menon, S., Liu, B., Hu, M. S., Longaker, M. T., and Lorenz, H. P. 2016. “Expansion and Hepatic Differentiation of Adult Blood-derived CD34+ Progenitor Cells and Promotion of Liver Regeneration after Acute Injury,” Stem Cells Translational Medicine 5(6):723-732; Moris, D., Vemnadakis. S., Papalampros. A., Vailas, M., Dimitrokallis, N., Petrou, A., and Dimitroulis, D. 2016. “Mechanistic Insights of Rapid Liver Regeneration after Associating Liver Partition and Portal Vein Ligation for Stage Hepatectomy,” World Journal of Gastroenterology 22(33):7613-7624; Kwon, Y. J., Lee, K. G., and Choi, D. 2015. “Clinical Implications of Advances in Liver Regeneration,” Clinical and Molecular Hepatology 21(1):7-13: Mao, S. A., Glorioso, J. M., and Nyberg, S. L. 2014. “Liver Regeneration,” Translational Research 163(4):352-362; Duncan, A. W. and Soto-Gutierrez, A. 2013. “Liver Repopulation and Regeneration: New Approaches to Old Questions,” Current Opinion in Organ Transplantation 18(2):197-202; Chistiakov, D. A. 2012. “Liver Regenerative Medicine: Advances and Challenges,” Cells, Tissues, Organs 196(4):291-312; Michalopoulos, G. K. 2011. “Liver Regeneration: Alternative Epithelial Pathways,” International Journal of Biochemistry and Cell Biology 43(2):173-179; Tanimizu, N. and Miyajima, A. 2007. “Molecular Mechanism of Liver Development and Regeneration,” International Review of Cytology 259:1-48; Fausto, N., Campbell, J. S., and Riehle. K. J. 2006. “Liver Regeneration,” Hepatology 43(2 Supplement 1):S45-553; Kountouras, J., Boura. P., and Lygidakis, N. J. 2001. “Liver Regeneration after Hepatectomy,” Hepatogastroenterology 48(38):556-562; Fausto, N. and Webber, E. M. 1993. “Control of Liver Growth,” Critical Reviews in Eukaryotic Gene Expression 3(2):117-135).

Where the chest is not an option, were regeneration not a problem, the upper abdomen would be the next best position. Aging tends to reduce the rate of regeneration following a partial hepatectomy but not the eventual volume to which the liver is restored. Provided the rate of regeneration in an elderly patient is decreased enough, what favorable factors the upper abdomen offers would be realized for a cohort in whom heart failure is prevalent (Hu, S., Jiang, S. S., Zhang, J., Luo. D., Yu, B., and 6 others 2019. “Effects of Apoptosis on Liver Aging,” World Journal of Clinical Cases 7(6):691-704; Lam, C. J., Rankin, M. M., King, K. B., Wang, M. C., Shook, B. C., and Kushner, J. A. 2019. “Glucagon Receptor Antagonist-stimulated α-Cell Proliferation is Severely Restricted with Advanced Age,” Diabetes 68(5):963-974; Li, M., Chu, Z., Tan, Z., Jin, Y., Xu. M., and Ji, Q. 2019. “Impact of Donor Age on Liver Regeneration and Function following Adult Living Donor Liver Transplantation,” Experimental and Therapeutic Medicine 17(5):3965-3970; Chen, G., Zhang, J., Sun, J., Wei. S., Chen, J., Ren, H., and Zhou, S. 2018. “Revisiting Partial Hepatectomy of Large Hepatocellular Carcinoma in Older Patients,” (Nature Research) Scientific Reports 8(1):14505; Erichsen, L., Beermann, A., Arauzo-Bravo, M. J.3. Hassan, M., Dkhil, M. A., and 4 others 2018. “Genome-wide Hypomethylation of LINE-1 and Alu Retroelements in Cell-free DNA of Blood is an Epigenetic Biomarker of Human Aging,” Saudi Journal of Biological Sciences 2018 25(6):1220-1226; Pibiri, M. 2018. “Liver Regeneration in Aged Mice: New Insights,” Aging (Albany, New York) 10(8):1801-1824; Saito, Y., Morine, Y., and Shimada, M. 2017. “Mechanism of Impairment on Liver Regeneration in Elderly Patients: Role of Hepatic Stellate Cell Function,” Hepatology Research 47(6):505-513; Zhong, H. H., Hu, S., Yu. B., Jiang, S. S., Zhang, J., and 7 others 2017. “Apoptosis in the Aging Liver,” Oncotarget 8(60):102640-102652; Pibiri, M., Sulas, P., Leoni. V. P., Perra, A., Kowalik. M. A., and 4 others 2015. “Global Gene Expression Profile of Normal and Regenerating Liver in Young and Old Mice,” Age (Dordrecht, Holland) 37(3):9796; Fan, H. L., Hsieh, C. B., Chang, W. C., Huang, S. H., Chan, D. C. 2014. “Advanced Age is Not a Contraindication for Liver Resection in Cases of Large Hepatocellular Carcinoma,” European Journal of Surgical Oncology 40(2):214-219; Schmucker, D. L. and Sanchez, H. 2011. “Liver Regeneration and Aging: A Current Perspective,” Current Gerontology and Geriatrics Research 2011:526379; Timchenko, N. A. 2009. “Aging and Liver Regeneration,” Trends in Endocrinology and Metabolism 20(4):171-176; Krupczak-Hollis, K., Wang, X., Dennewitz, M. B., and Costa, R. H. 2003. “Growth Hormone Stimulates Proliferation of Old-aged Regenerating Liver through Forkhead Box m1b,” Hepatology 38(6):1552-1562; Wang, X., Krupczak-Hollis, K., Tan, Y., Dennewitz, M. B., Adami, G. R., and Costa, R. H. 2002. “Increased Hepatic Forkhead Box MIB (FoxM1B) Levels in Old-aged Mice Stimulated Liver Regeneration through Diminished p27Kip1 Protein Levels and Increased Cde25B Expression,” Journal of Biological Chemistry 277(46):44310-44316; Wang, X., Quail. E., Hung, N. J., Tan, Y., Ye, H., and Costa, R. H. 2001. “Increased Levels of Forkhead Box MIB Transcription Factor in Transgenic Mouse Hepatocytes Prevent Age-related Proliferation Defects in Regenerating Liver,” Online Proceedings of the National Academy of Sciences of the United States of America 98(20):11468-11473).

By the same token, a partial hepatectomy in an elderly patient, unfortunately often associated in the literature, hence, conflated with, the removal of hepatocellular carcinoma or intrahepatic cholangiocarcinoma, for example, might result in complications that would not arise in a young patient. Additionally, advanced age does not equate to frailty, and involving every organ system, frailty is itself ill-defined. Both advanced age and frailty are too multifactorial to extricate the relationship between the rate of liver regeneration and the overall condition of the patient in a specific sense and these to the morbidity and mortality that might follow a partial hepatectomy (see also, for example, Dili, A., Lebrun. V., Bertrand, C., and Leclercq, I. A. 2019. “Associating Liver Partition and Portal Vein Ligation for Staged Hepatectomy: Establishment of an Animal Model with Insufficient Liver Remnant,” Laboratory Investigation 99(5):698-707; Inoue, Y., Fujii, K., Ishii, M., Kagota. S., Tomioka, A., and 14 others 2019. “Volumetric and Functional Regeneration of Remnant Liver after Hepatectomy,” Journal of Gastrointestinal Surgery 23(5):914-921; Panayi, A. C., Orkaby, A. R., Sakthivel. D., Endo, Y., Varon, D., and 6 others 2019. “Impact of Frailty on Outcomes in Surgical Patients: A Systematic Review and Meta-analysis,” American Journal of Surgery 218(2):393-400; Peng. W., Li, J. W., Zhang, X. Y., Li, C., Wen, T. F., Yan, L. N., and Yang, J. Y. 2019. “A Novel Model for Predicting Posthepatectomy Liver Failure in Patients with Hepatocellular Carcinoma,” Online, PLoS [Public Library of Science] One 14(7):e0219219; Schwarz, C., Plass, I., Fitschek, F., Punzengruber, A., Mittlbock, M., and 6 others 2019. “The Value of Indocyanine Green Clearance Assessment to Predict Postoperative Liver Dysfunction in Patients Undergoing Liver Resection,” (Nature Research) Scientific Reports 9(1):8421; Takemura, N. and Kokudo, N. 2019. “Do We Need to Shift from Dye Injection to Fluorescence in Respective Liver Surgery?,” Surgical Oncology July 19. pii: S0960-7404(19) 30316-0; Xia. Y., Zhang, L., Zhao. Y., Liu X., Cai. L., and 3 others 2019. “Gallium-68-labelled Indocyanine Green as a Potential Liver Reserve Imaging Agent” Contrast Media and Molecular Imaging 2019:4201353; Asenbaum, U., Kaczirek. K., Ba-Ssalamah, A., Ringl. H., Schwarz, C., and 4 others 2018. “Post-hepatectomy Liver Failure after Major Hepatic Surgery: Not Only Size Matters,” European Radiology 28(11):4748-4756; Chen, G., Zhang, J., Sun, J., Wei, S., Chen, J., Ren, H., and Zhou, S. 2018. “Revisiting Partial Hepatectomy of Large Hepatocellular Carcinoma in Older Patients,” (Nature Research) Scientific Reports 8(1):14505; Chuang, Y. H., Ou, H. Y., Lazo, M. Z., Chen, C. L., Chen, M. H., Weng. C. C., and Cheng, Y. F. 2018. “Predicting Post-hepatectomy Liver Failure by Combined Volumetric, Functional MR Image and Laboratory Analysis,” Liver International 38(5):868-874; Costa, A. F., Tremblay St-Germain, A., Abdolell. M., Smoot, R. L., Cleary, S., and Jhaveri, K. S. 2018. “How Do Different Indices of Hepatic Enhancement with Gadoxetic Acid Compare in Predicting Liver Failure and Other Major Complications after Hepatectomy?,” Journal of Computer Assisted Tomography 42(3):380-386; Gupta, M., Choudhury, P. S., Singh, S., and Hazarika, D. 2018. “Liver Functional Volumetry by Tc-99m Mebrofenin Hepatobiliary Scintigraphy before Major Liver Resection: A Game Changer,” Indian Journal of Nuclear Medicine 33(4):277-283; Kim, D. K., Choi, J. I., Choi, M. H., Park, M. Y., Lee, Y. J., Rha. S. E., and Jung, S. E. 2018. “Prediction of Posthepatectomy Liver Failure: MRI with Hepatocyte-specific Contrast Agent versus Indocyanine Green Clearance Test,” American Journal of Roentgenology 211(3):580-587; Rahnemai-Azar, A. A., Cloyd, J. M., Weber, S. M., Dillhoff, M., Schmidt, C., Winslow, E. R., and Pawlik, T. M. 2018. “Update on Liver Failure following Hepatic Resection: Strategies for Prediction and Avoidance of Post-operative Liver Insufficiency,” Journal of Clinical and Translational Hepatology 6(1):97-104; Ray, S., Mehta, N. N., Golhar, A., and Nundy, S. 2018. “Post Hepatectomy Liver Failure-A Comprehensive Review of Current Concepts and Controversies,” Annals of Medicine and Surgery (London, England) 34:4-10; Shen, Y. N., Zheng, M. L., Guo, C. X., Bai, X. L., Pan. Y., Yao. W. Y., and Liang, T. B. 2018. “The Role of Imaging in Prediction of Post-hepatectomy Liver Failure,” Clinical Imaging 52:137-145; Verma, B. K., Subramaniam, P., and Vadigepalli, R. 2018. “Modeling the Dynamics of Human Liver Failure Post Liver Resection,” Processes (Basel, Switzerland) 6(8). pii: 115; Andreatos, N., Amini, N., Gani. F., Margonis, G. A., Sasaki, K., and 6 others 2017. “Albumin-Bilirubin Score: Predicting Short-term Outcomes Including Bile Leak and Post-hepatectomy Liver Failure following Hepatic Resection,” Journal of Gastrointestinal Surgery 21(2):238-248; Costa, A. F., Tremblay, St-Germain, A., Abdolell, M., Smoot, R. L., Cleary, S., and Jhaveri, K. S. 2017. “Can Contrast-enhanced MRI with Gadoxetic Acid Predict Liver Failure and Other Complications after Major Hepatic Resection?,” Clinical Radiology 72(7):598-605; Gani, F., Cerullo, M., Amini, N., Buettner, S., Margonis, G. A., and 3 others, 2017. “Frailty as a Risk Predictor of Morbidity and Mortality following Liver Surgery,” Journal of Gastrointestinal Surgery 21(5):822-830; Ibis, C., Albayrak, D., Sahiner, T., Soytas, Y., Gurtekin, B., and Sivrikoz, N. 2017. “Value of Preoperative Indocyanine Green Clearance Test for Predicting Post-hepatectomy Liver Failure in Noncirrhotic Patients,” Medical Science Monitor 23:4973-4980; Kim, B. J., Tzeng. C. D., Cooper, A. B., Vauthey, J. N., and Aloia, T. A. 2017. “Borderline Operability in Hepatectomy Patients is Associated with Higher Rates of Failure to Rescue after Severe Complications,” Journal of Surgical Oncology 115(3):337-343; Lodewick, T. M., Alizai, P. H., van Dam, R. M., Roeth, A. A. J., Schmeding, M., and 5 others 2017. “Effect of Age on Liver Function in Patients Undergoing Partial Hepatectomy,” Digestive Surgery 34(3):233-240; Rassam, F., Olthof, P. B., Bennink, R. J., and van Gulik, T. M. 2017. “Current Modalities for the Assessment of Future Remnant Liver Function,” Visceral Medicine 33(6):442-448; Shi, J.ndH., Hammarstrom, C., Grzyb. K., and Line, P. D. 2017. “Experimental Evaluation of Liver Regeneration Patterns and Liver Function following ALPPS [Associating Liver Partition and Portal vein ligation (PVL) for Staged hepatectomy],” British Journal of Surgery Open 1(3):84-96; Lin, H. S., Watts, J. N., Peel. N. M., and Hubbard, R. E. 2016. “Frailty and Post-operative Outcomes in Older Surgical Patients: A Systematic Review,” Online, BioMed Central Geriatrics 16(1):157; Lafaro, K., Buettner, S., Maqsood, H., Wagner, D., Bagante, F., and 4 others 2015. “Defining Post Hepatectomy Liver Insufficiency: Where Do We Stand?,” Journal of Gastrointestinal Surgery (New York, New York) 19(11):2079-2092; Eshkenazy, R., Dreznik, Y., Lahat, E., Zakai, B. B., Zendel, A., and Ariche, A. 2014. “Small for Size Liver Remnant following Resection: Prevention and Management,” Hepatobiliary Surgery and Nutrition 3(5):303-312; Fan, H. L., Hsieh, C. B., Chang, W. C., Huang, S. H., Chan, D. C., and 3 others 2014. “Advanced Age is Not a Contraindication for Liver Resection in Cases of Large Hepatocellular Carcinoma,” European Journal of Surgical Oncology 40(2):214-219; Tzeng. C. W., Cooper, A. B., Vauthey, J. N., Curley, S. A., and Aloia, T. A. 2014. “Predictors of Morbidity and Mortality after Hepatectomy in Elderly Patients: Analysis of 7621 NSQIP Patients,” Hepato Pancreato Biliary Association Journal 16(5):459-468; Wibmer, A., Prusa, A. M., Nolz, R., Gruenberger, T., Schindl, M., Ba-Ssalamah, A. 2013. “Liver Failure after Major Liver Resection: Risk Assessment by Using Preoperative Gadoxetic Acid-enhanced 3-TMR [3 Tesla magnetic resonance imaging] Imaging,” Radiology 269(3):777-786; Guglielmi, A., Ruzzenente, A., Conci, S., Valdegamberi, A., and lacono, C. 2012. “How Much Remnant is Enough in Liver Resection?,” Digestive Surgery 29(1):6-17; Schneider, P. D. 2004. “Preoperative Assessment of Liver Function,” Surgical Clinics of North America 84(2):355-373).

In the elderly, even elective partial hepatectomy to remove cysts or benign tumors does not afford a clear picture as to morbidity and mortality directly attributable to the surgery (Cady, B., Bonneval. M., and Fender, H. R. Jr. 1979. “Elective Hepatic Resection,” American Journal of Surgery 137(4):514-521).

The active suppression of liver regeneration responsive to a partial hepatectomy undertaken to create space for a second heart, even when the alternative positions delineated above must be ruled out, must be rejected. In such an extreme and improbable circumstance, the patient should be sustained on liver dialysis. The ability even with directly pipelined drug targeting and impasse-jackets as described in U.S. Pat. No. 11,389,171, entitled Integrated System for the Infixion and Retrieval of Implants, to interfere with its regenerative resources without impairing the wider normal physiology of the remaining liver may be impossible (see, for example, Alison, M. R. and Lin, W. R. 2016. “Regenerating the Liver: Not So Simple After All?,” Faculty 1000 Research 5. pii: F1000 Faculty Rev-1818; Liu, B., Bell. A. W., Paranjpe. S., Bowen, W. C., Khillan, J. S., and 3 others 2010. “Suppression of Liver Regeneration and Hepatocyte Proliferation in Hepatocyte-targeted Glypican 3 Transgenic Mice,” Hepatology 52(3):1060-1067).

Regardless of the site selected, the trauma of incision is deferred until after the donor heart has been fully connected with lines short as possible to the native or the orthotopic or conventional heterotopic duo heart transplanted earlier, stabilized in position adjacent the prospective entry incision, and the components of the control system, to include the servovalves and execution of the prescription-program, fully tested. Angioplasty, conventional or as delineated in U.S. Pat. No. 11,389,171, entitled Integrated System for the Infixion and Retrieval of Implants, is employed to remove any atheromatous blockages in the coronary arteries of both hearts which could otherwise induce an arrhythmia following transplantation to complicate a differential diagnosis as to a source attributable instead to an improper apportionment of output or venous return in either or both hearts.

If the second heart is to be placed infrarenally, or in the lower abdomen, the protective heart cage described below in section 2b(1)(b)(b6), entitled Heart Cage for the Infrarenal Abdominal Positioning of a Second Heart, is applied while the heart remains extracorporeal. Solenoid-driven valves lacking the continuous adjustability required to perform metered transplantation and maintain optimal performance of the two hearts in coordination following transplantation, such a double or added heart transplant requires vascular servovalves to connect the corresponding vessels of the orthotopic and donor hearts.

The implanted microcontroller or microprocessor dynamically adjusts the servovalves of either heart to achieve the optimal performance of each and the two as a functional unit. In pediatric heart failure not yet having resulted in secondary sequelae typically renal or hepatic, a relatively simple microcontroller monitors, that is, receives sensor inputs, and responds by adjusting as appropriate, the implant drug reservoir outlet pump or drip stopcock and/or the servovalve on each vessel. More specifically, the microcontroller also controls the dispensing of the drug or drugs specified in the prescription-program through the one or more accessory channels of each servovalve. Having thus interdicted the development of secondary impairments, the need for control by a microprocessor suitable for dealing with comorbid disease has been precluded.

The major organs—indeed, all the tissues of the body—functionally interdependent, the patient with acute decompensated heart failure will usually present with associated renal, hepatic, and other impairments (see, for example, Al-Jarallah, M., Rajan, R., Al-Zakwani, I., Dashti, R., Bulbanat, B., and 13 others 2019. “Incidence and Impact of Cardiorenal Anaemia Syndrome on All-cause Mortality in Acute Heart Failure Patients Stratified by Left Ventricular Ejection Fraction in the Middle East,” European Society of Cardiology Heart Failure 6(1):103-110; Borovac, J. A., Novak, K., Bozic, J., and Glavas, D. 2019. “The Midrange Left Ventricular Ejection Fraction (LVEF) is Associated with Higher All-cause Mortality during the 1-year Follow-up Compared to Preserved LVEF among Real-world Patients with Acute Heart Failure: A Single-center Propensity Score-matched Analysis,” Džubur, A., Ejubović, M., Fajkić, A., Dervišević, A., Durak Nalbantić. A., and 3 others 2019. “The Serum Triglyceride to High-density Lipoprotein (HDL) Ratio in Patients with Acute Coronary Syndrome with and without Renal Dysfunction,” Medicinski Glasnik [Medical Herald] (Zenica, Bosnia-Herzegovina) 16(1):28-34; Peyracchia, M., Scacciatella, P., Conrotto, F., Meynet, I., Biava, L. M., and 4 others 2018. “Impact of Chronic Kidney Disease on Mortality in Patients with ST-segment Elevation Myocardial Infarction Treated with Primary Percutaneous Coronary Intervention. A Long-term Single-center Mortality Study,” Minerva Cardioangiologica 66(1):6-15; Wakabayashi, K., Ikeda, N., Kajimoto, K., Minami, Y., Keida, T., and 7 others 2018. “Trends and Predictors of Non-cardiovascular Death in Patients Hospitalized for Acute Heart Failure,” International Journal of Cardiology 250:164-170; Hsu, J. J., Ziaeian, B., and Fonarow, G. C. 2017. “Heart Failure with Mid-range (Borderline) Ventricular Ejection Fraction: Clinical Implications and Future Directions,” Journal of the American College of Cardiology. Heart Failure 5(11):763-771; Li, G., Qi. G., Zhang. B., Zhou, B., Ma, B., and 6 others 2017. “The Dose-response Association between Estimated Glomerular Filtration Rate and Prognosis of Patients with ST-segment Elevation Myocardial Infarction from Rural Areas of China's Liaoning Province,” Medicine (Baltimore, Maryland) 96(52):e9508; Martinez, F., Martinez-Ibanez, L., Pichler, G., Ruiz, A., and Redon, J. 2017. “Multimorbidity and Acute Heart Failure in Internal Medicine,” International Journal of Cardiology 232:208-215; Wakabayashi, K., Sato, N., Kajimoto, K., Minami, Y., Mizuno, M., and 7 others 2017. “Incidence and Predictors of In-hospital Non-cardiac Death in Patients with Acute Heart Failure,” European Heart Journal. Acute Cardiovascular Care 6(5):441-449; Moreira-Silva, S., Urbano, J., Moura, M. C., Ferreira-Coimbra, J., Bettencourt, P., and Pimenta, J. 2016. “Liver Cytolysis in Acute Heart Failure: What Does It Mean? Clinical Profile and Outcomes of a Prospective Hospital Cohort,” International Journal of Cardiology 221:422-427; Hu, L. H., Zhang, L. J., Jin, Z. T., Yang, W., Zhang, L. N., and Lu, C. Y. 2015. “Analysis of the Clinical Characteristics of Patients with Acute Coronary Syndrome in Different States of Renal Function,” West Indian Medical Journal 64(4):357-361; Laribi, S. and Mebazaa, A. 2014. “Cardiohepatic Syndrome: Liver Injury in Decompensated Heart Failure,” Current Heart Failure Reports 11(3):236-240; Widimsky, P. and Rychlik, I. 2010. “Renal Disease and Acute Coronary Syndrome,” Heart 96(1):86-92; additional references cited in sections 2a. General. (1). Congenital Defects of the Heart and Other Organs: The Importance of Prompt Correction; a. General. (3). Remote Graft Organ Preservation; b. Vascular Valves and Servovalves in Vascular Diversion, (1). Intercorporeal Diversion of Blood Flow; b. Vascular Valves and Servovalves in Vascular Diversion; and (b). Metered compound vascular bypass, or sudden switched-organ transplantation, (b2), entitled Antepartum Compound vascular bypass, or sudden switched-organ Transplantation of the Heart.

In this circumstance, the implanted control device is not a simpler microcontroller suitable for governing the diagnosis and treatment of a morbidity as if it were isolated, but rather a microprocessor executing a hierarchical prescription-program. In such a program, each comorbidity is assigned to an arm or channel of control, each arm responding to in situ sensor inputs and—in accordance with the prescription-programmed-administering therapeutic treatment responsive to a progressively expanded zone of physiological inclusion. That is, coordination among the arms is achieved by referral upwards through intervening cross-arm control nodes to the overall or comprehensive-level processing of the master control microprocessor executing a prescription-program.

The sufficiency of the response will then depend upon the sufficiency of the sensors implanted to provide diagnostic data and the sufficiency of the therapeutic means implanted, of which both are prescribed to include any known or likely to be needed. The treatment of comorbid disease will typically include the physiological sensors essential to detect the manifestations, or signs and symptoms, of each morbidity, and the drug delivery and electrostimulatory actuators essential to respond to these. The master controller will typically coordinate its action with one—or following a double heart transplant-two cardioverter defibrillators as well as other neuromodulators, such as nondefibrillation electrostimulators-.

At the higher, or master control level in the hierarchy, treatment of the comorbidities is coordinated to achieve homeostatic optimization of the patient as a whole. While no level of acumen in the design of an automatic disorder response system can overcome impairment beyond a certain degree, the realization from the local to the comprehensive level of automatic immediate response to deviations from the normal, should sustain a large number of patients who would otherwise die, and the stabilization afforded will often maintain the patient in a condition that allows the end stage organ or organs to be replaced. As the preceding sections delineate, the automatic response system can not only administer this replacement, but can continuously adjust to the overall condition of the patient at each stage. That is, optimize health prior to, during, and following transplantation, a transcatheteric or transluminal, or any other procedure.

In conventional heterotopic transplantation, the misapportionment of blood between the native and an undersized added heart is a frequent cause for graft failure. The continuous dynamic adjustment of each vessel valve within the context of all the valves functioning as a unit eliminates this problem, significantly enhancing the usability of undersized hearts and therewith, expanding the zone of hearts usable for transplantation. Separate, the transplant heart is separately targetable for the administration of immunosuppressant and anti-inflammatory medication. In an orthotopic heart transplant using the method described above in section 2b(1)(b) entitled Metered compound vascular bypass, or sudden switched-organ transplantation, which uses vascular servovalves to create a compound vascular bypass around the native to the donor heart, the native heart is removed and the donor heart placed in the chest of the recipient with the valves having been fully extended to divert all blood through the new heart.

Sufficient space in the chest usually wanting, when the native heart is to be preserved and a second heart situated heterotopically, such as in the abdominal cavity, infrarenally, or within pelvis, the valves of the native heart are brought to half extension thus dividing the supply into and drainage out of the native heart to one half, the other half passed through the added heart. The valves in the added heart remain fully extended. The half-extended positioning of the valves in the native heart is nominal, contingent upon its size and ventricular absolute stroke volume compared to the donor heart. Whether transcatheteric or transluminally or in an open surgical field, a postprocedural need for surgical access to either heart is uncomplicated by fusion of the native or earlier orthotopically placed heart to that added as in conventional heterotopic transplantation.

That is, the microcontroller dynamically adjusts each valve as necessary to obtain the optimum coordination for each and for both of the two hearts functioning as a unit, thus minimizing if not eliminating deviations in the volumes or pressures in the chambers as might precipitate a dysrhythmia. If when performing optimally, there exist sites of hypertension or hypervolemia, then these can be ameliorated through localized diversion of excess blood to a nearby large vessel of the same kind as addressed below in section 2b(2)(h)(h4), entitled The ‘Tuned’ Shunt in the Relief of Vascular Arterial Hypertension.

The microcontroller program, fed inputs from sensors along the major vessels of both hearts then seeks, whether through sensor reading programming or through trial and error, to optimize the function of both hearts by adjusting the valves as necessary. The major vessels of both hearts valved, the degree to which flow through any one can be diverted can be varied from 0-100 percent; however, the valves are designed to be fail-safe in that only the relative proportion of flow diverted is variable-even in the improbable event of a malfunction, the valves cannot obstruct flow.

The advantages of continuous dynamic adjustability of each valve should the need arise are sufficient to restrict the use of solenoidal valves for heart transplantation to emergency use, especially in undeveloped countries where the higher cost of servovalves is a significant consideration. Blood volumes and pressures considerable, the liver excepted, this factor is particularly important in heart transplantation, but less so in the transplantation of most other organs and glands, for example. Solenoidal valves do, however, incorporate accessory channels, or sidelines, which allow the direct pipeline targeting of drugs to each vessel, making these adequate for transplanting other organs and glands.

Not recommended and only to clarify the extent of control overflow made possible, it would even be possible, for example, to combine the right heart of one heart with the left heart of the other (Magder, S. 2016. “Volume and Its Relationship to Cardiac Output and Venous Retum,” Critical Care (London, England) 20:271; Elefteriades, J., Lovoulos, C., Edwards, R., Tittle, S., Riley, T., and 3 others 2003. “Novel Technique for Isolated Accessory Right Heart Transplantation for Congenital Heart Disease,” Journal of Thoracic and Cardiovascular Surgery 125(6):1283-1290, (Op cit. above under 1. Background of the Invention, a. General); Mosca, S. M., Escudero, E., Gelpi, R. J., Kosoglov, A. T., Rinaldi, G. J., and Cingolani, H. E. 1981. “Myocardial Flow Distribution. II: Empty Beating Heart, Ventricular Fibrillation and Cardiac Arrest,” Archives Internationales de Physiologie et de Biochimie 89(4):357-364).

The condition of one side of the heart beating while the other is beating without blood is novel. Even though the overall movement of blood between the two hearts would achieve substantially normal perfusion, because the conduction system in each heart is substantially autonomous, the result would likely be for both to fibrillate or even go into arrest, necessitating the prepositioning of a cardioverter defibrillator on each. For one side of the heart to remain bloodless would change the balance of forces between the sides, and while the difference would not accede to wrenching, continuation beyond a certain duration would likely result in injury to the empty side.

However, over a short term, a bloodless field beating in the beating heart which eliminated the need for general anesthesia and the risk of postperfusion syndrome would allow open or transcatheteric or transluminal surgery or visual examination of the inside of the chambers. The drug delivery catheter or viewing device is connected to the outer surface of the beating heart can be kept at the correct radial angle by connection with a nonjacketing side-entry connector as described in U.S. Pat. No. 11,013,858.

The addition to the native of a second intact heart where the apportionment of the total volume of recipient blood can be continuously adjusted between the two on a vessel by vessel basis to optimize the performance of the hearts as cooperative, or combined, overcomes a major drawback in conventional heterotopic transplantation in that the addition of a second heart too small frequently results in failure (references follow in this section). Furthermore, the accessory channel or channels in each side-entry diversion servovalve allows the direct targeting to each vessel of drugs such as immunosuppressant or antihypertensive when the need therefore is indicated by sensors incorporated into each servovalve.

By averting this eventuality, the zone of suitable transplant hearts is materially expanded, eliminating many deaths of patients who die while waiting for a transplant. The addition of a second heart in the manner indicated is fundamentally different from conventional heterotopic transplantation in that each heart, while cooperating with the other each under its own power to attain as close to the normal function of one good heart as possible, remains completely intact and separately addressable for the targeting of drugs. Indeed, either side of either heart, and every inflow and outflow vessel of either heart can be targeted thus.

Servovalves such as those described herein having been unavailable to combine two hearts as indicated, the subject of two separate intact hearts connected in series is addressed in the literature only when a failed heterotopic transplant forced an orthotopic transplant where the graft was kept in place (see, for example, Smith, R. D. Jr., Radovancevic, B., Gregoric, I. D., Cohn, W. E., and Frazier, O. H. 2006. “Orthotopic Retransplantation in Heterotopic Transplant Recipients: 3 Case Reports,” Journal of Heart and Lung Transplantation (New York, New York) 25(2):245-247; Fiorelli, A. I., Coelho, G. H., Lima, J. L., Lourenço, D. D., Gutierres, P., and 4 others 2009, Op cit. above in section 2b(1)(b)(b3), entitled Neonatal and Infant Compound Vascular Bypass, or Sudden Switched-organ Transplantation of the Heart).

Primarily because of the surgical trauma involved in conventional heart transplantation, the few instances where double heartedness is found in the literature does not apply to nonfusion heterotopic, or double heart transplantation as described herein. Such an arrangement warrants study both without and with implanted means for synchronization. The conduction systems substantially autonomous within each, should one become dysrhythmic, it has not been established whether the other would likewise or would instead assist the other back to recover to a normal rhythm. Except where size mismatch is extreme, for the donor heart not to assist and even allow the native heart to recover is exceptional.

In a conventional heterotopic heart transplant, evidence exists to suggest that the latter is true (Housmans, P. R., Rietman, G. W., Wellens, F., Verbeke, J., and Nollet, G. 1996. “Ventricular Dysrhythmia in the Native Heart after Heterotopic Heart Transplantation: Diagnosis with Selective Electrocardiography,” Anesthesiology 85(4):936-939; Neerukonda, S. K., Schoonmaker, F. W., Nampalli, V. K., and Narrod, J. A. 1992. “Ventricular Dysrhythmia and Heterotopic Heart Transplantation,” Journal of Heart and Lung Transplantation (New York, New York) 11(4 Part 1):793-796; Kotliar, C., Smart, F. W., Sekela, M. E., Pacifico, A., Pratt, C. M., and 3 others 1991. “Heterotopic Heart Transplantation and Native Heart Ventricular Arrhythmias,” Annals of Thoracic Surgery 51(6):987-991). Whether one heart beating normally when the other is not would sustain the recipient would likely depend upon the severity of the arrhythmia, one ventricular, for example, more menacing than one atrial.

If the cause is internal to one of the hearts, such as a previously undetected block, then the other heart is exposed to the desynchronizing consequence but not to the source of interference. Double heart heterotopic heart transplantation as described herein eliminates the numerous insults to both native and transplant hearts sustained in conventional heart transplantation whether orthotopic or heterotopic with the surgical merging of the native with a donor heart. These insults include the shock of death, bisection, ischemia-reperfusion injury, the need for cardiopulmonary support with cross-clamping, cardioplegia, the use of thoracic regional rather than general anesthesia, and therewith, the risk of postperfusion syndrome. A key advantage of double heart transplantation using the switch technique over the conventional heterotopic or Barnard-Losman conjoined configuration is that the native, or intact in-line, and assist, or intact off-line, hearts remain separate.

Once the hearts in a conventional heterotopic transplant have been surgically joined, to reverse the procedure in order to recover and repair either heart would repeat the considerable surgical trauma undertaken to create the merger in the first place, making the prospect misconceived. That is, even if the temporary ventricular assistance provided by the second heart allowed the native heart to recover, to undo the extensive anastomoses and dissection that injured both hearts in order to remove it would likely preclude any benefit in the recovery.

Thus, mention in the literature of reversal is rare and dictated by necessity where a considerably undersized graft in a heterotopic transplant was retained alongside a second orthotopically placed heart (Smith, R. D. Jr., Radovancevic, B., Gregoric, I. D., Cohn, W. E., and Frazier, O. H. 2006, Op cit. Op cit. above in section 2b(1)(b)(b3), entitled Neonatal and Infant Compound Vascular Bypass, or Sudden Switched-organ Transplantation of the Heart, and in this section; Fiorelli, A. I., Coelho, G. H., Lima, J. L., Lourenço, D. D., Gutierres, P., and 4 others 2009, Op cit. above also in section 2b(1)(b)(b3), and in this section) and the absence from the literature of a recommended procedure to reverse the combination is clear. Moreover, the extended use of a left ventricular assist device associated with left ventricular atrophy and right heart failure, if the heterotopic transplant as a whole or in either of its constituents malfunctions, the best option is to replace it with an orthotopic heart transplant.

If for any reason, either the native or added heart must be treated separately from the other or removed, having been established using a switch technique, this is readily accomplished. Surgically, functionally, and medically, the hearts are separately addressable. If the native heart must be removed, the venous return and great arterial valves are switched to pass all blood directly through the added or donor heart; if that heart must be removed, then the valves are switched to direct all blood through the native or earlier transplanted heart.

Since flow is unidirectional from venous return to the native heart then passed through the donor heart and great artery flow from the donor to the native heart, there is never the need to reverse this direction. The relaive proportion of blood passed to either heart adjustable from 0 to 100 percent, the setpoints for the servovalves passes 50 percent to each, which is adjustable to accommodate a size mismatch. However, with a significant difference in size and/or ventricular absolute stroke volume, the hearts adapt to their altered context, primarily by progressive relaxation by the larger left ventricle. As the time until this adaptation is complete can be marked by convulsion and coma, the selective targetability of the hearts for drugs is important.

The accessory channels in basic ductus side-entry jackets as described in U.S. Pat. No. 11,759,186 allow targeting drugs to the coronary arteries of either heart, the servovalve-jackets allow targeting any in- or outflow vessel with medication, and the rate flow through either or both right and left hearts can be adjusted. This would then force a retransplantation or the placement of mechanical devices of which none could sustain a younger patient to the end of life. Nevertheless, the addition even to the conventional heterotopic model of an implanted system of the kind that with respect to the double heart model is presupposed from the outset, would improve outcomes with the merged configuration.

But while the diagnostic sensors and drug release capabilities provided by the accessory channels of the system could be applied, the function served by the diversion valves in a double heart transplant will have been preempted by the conjoined configuration. For the double heart configuration, not only do the valve-jackets on the major blood vessels incorporate accessory channels to allow the direct targeting of drugs, but continuously variable blood diversion valves can be used separately or in coordination with diversion to allow the direct, selective and strategic, targeting to any supply region of immunosuppressant, anti-inflammatory, and/or antimicrobial drugs, avoiding systemic dispersion with its undesirable side effects, and considerably reducing the risk of mid- and postprocedural thromboembolism as well as cerebral or myocardial infarction.

The valves allow flow and blood pressure through any vessel to be continuously varied from outside the body. In fundamental contrast to conventional procedures which inflict extensive incisions and dissection on both the native and donor hearts provoking mid- and postprocedural arrhythmias, neither heart is entered. Lengthy incisions that come to underlie other structures once the conjoined hearts are placed in the chest become difficult to access for repair should the anastomoses leak, for example.

In both a conventional heterotopic and a double heart transplant using the switch technique, an inability of both hearts combined to generate sufficient power of ejection can be ameliorated by the direct targeting to the coronary arteries of either or both hearts of a positive inotrope in a relatively high dose without the numerous adverse side effects associated with its systemic dispersion and without affecting either heart if not targeted.

If uptake of a drug targeted to one of the hearts is not complete and a residue should be prevented from continued flow to the other heart, then as described in U.S. Pat. No. 11,389,171, entitled Integrated System for the Infixion and Retrieval of Implants, a magnetized impasse- or trap-jacket is used to draw it with bound superparamagnetic carrier out from the venous outflow of the target organ. Mechanically extracted thus where a buildup of the carrier would accumulate threatening blockage, a water soluble carrier is used. Whether the mechanical impasse- or trap-jacket or a reversal agent delivered from the reservoir through a ductus side-entry jacket is used, the jackets of either or both types are applied to the stump or stumps of the native or graft organ adjacent to the diversion servovalves.

If a reversal agent is available, then a ductus side-entry jacket as described in U.S. Pat. No. 11,759,186, entitled Ductus Side-entry Jackets and Prosthetic Disorder Response Systems is used to release the reversal agent into the organ or graft organ venous outflow. For use with radionuclides, the small implanted flat drug reservoirs positioned in the pectoral region and impasse- or ductus side-entry jackets are radiation shielded. Where the agent is radioactive as well as otherwise injurious to nontargeted tissue such as a radionuclide chemotherapeutic, tightly defined targeting to include the complete eradication of the agent may warrant the use of these measures in combination.

Water soluble superparamagnetic carrier technology is highly developed (see, for example, Ansari, S. A. M. K., Ficiara, E., Ruffinatti, F. A., Stura, I., Argenziano, M., and 4 others 2019. “Magnetic Iron Oxide Nanoparticles: Synthesis, Characterization and Functionalization for Biomedical Applications in the Central Nervous System,” Materials (Basel, Switzerland) 12(3). pii: E465; Arias, L. S., Pessan, J. P., Vieira, A. P. M., Lima, T. M. T., Delbem, A. C. B., and Monteiro, D. R. 2018. “Iron Oxide Nanoparticles for Biomedical Applications: A Perspective on Synthesis, Drugs, Antimicrobial Activity, and Toxicity,” Antibiotics (Basel, Switzerland) 7(2). pii: E46; Davis, K., Vidmar, M., Khasanov, A., Cole, B., Ghelardini, M., and 5 others 2018. “The Effect of Post-synthesis Aging on the Ligand Exchange Activity of Iron Oxide Nanoparticles,” Journal of Colloid and Interface Science 511:374-382; Shen, L., Li, B., and Qiao, Y. 2018. “Fe₃O₄ Nanoparticles in Targeted Drug/Gene Delivery Systems,” Materials (Basel, Switzerland) 11(2). pii: E324; Zhu, N., Ji, H., Yu, P., Niu, J., Farooq, M. U., and 4 others 2018. “Surface Modification of Magnetic Iron Oxide Nanoparticles,” Nanomaterials (Basel, Switzerland) 8(10). pii: E810; Hu, L., Yang, Z., Wang, Y., Li, Y., Fan, D., and 3 others 2017. “Facile Preparation of Water-soluble Hyperbranched Polyamine Functionalized Multiwalled Carbon Nanotubes for High-efficiency Organic Dye Removal from Aqueous Solution,” (Nature Research) Scientific Reports 7(1):3611; Hu, L., Li, Y., Zhang, X., Wang, Y., Cui, L., and 4 others 2016. “Fabrication of Magnetic Water-soluble Hyperbranched Polyol Functionalized Graphene Oxide for High-efficiency Water Remediation,” (Nature Research) Scientific Reports 6:28924; Wu, W., Wu, Z., Yu, T., Jiang, C., and Kim, W. S. 2015. “Recent Progress on Magnetic Iron Oxide Nanoparticles: Synthesis, Surface Functional Strategies, and Biomedical Applications,” Science and Technology of Advanced Materials 16(2):023501; Majeed, I. M., Lu, Q, Yan, W., Li, Z., Hussain, I., and 4 others 2013. “Highly Water-soluble Magnetic Iron Oxide (Fe₃O₄) Nanoparticles for Drug Delivery: Enhanced in Vitro Therapeutic Efficacy of Doxorubicin and MION [monocrystalline iron oxide nanoparticles] Conjugates,” Royal Society of Chemistry Journal of Materials Chemistry Part B 22(1):2874-2884; Guardia, P., Di Corato, R., Lartigue, L., Wilhelm, C., Espinosa, A., and 4 others 2012. “Water-soluble Iron Oxide Nanocubes with High Values of Specific Absorption Rate for Cancer Cell Hyperthermia Treatment,” American Chemical Society Nano 6(4):3080-3091; Wang, L., Li, J., Jiang, Q., and Zhao, L. 2012. “Water- soluble Fe₃O₄ Nanoparticles with High Solubility for Removal of Heavy-metal Ions from Waste Water,” Royal Society of Chemistry Dalton Transactions (Cambridge, England) 41(15):4544-4551; Lee, J. E., Lee, N., Kim, T., Kim, J., and Hyeon, T. 2011. “Multifunctional Mesoporous Silica Nanocomposite Nanoparticles for Theranostic Applications,” (American Chemical Society)Accounts of Chemical Research 44(10):893-902; Xiao, L., Li, J., Brougham, D. F., Fox, E. K., Feliu, N., and 7 others 2011. “Water-soluble Superparamagnetic Magnetite Nanoparticles with Biocompatible Coating for Enhanced Magnetic Resonance Imaging,” American Chemical Society Nano 5(8):6315-6324; Xu, Y., Qin, Y., Palchoudhury, S., and Bao, Y. 2011. “Water-soluble Iron Oxide Nanoparticles with High Stability and Selective Surface Functionality,” Langmuir 27(14):8990-8997; De Angelis, F., Pujia, A., Falcone, C., laccino, E., Palmieri, C., and 8 others 2010. “Water Soluble Nanoporous Nanoparticle for in Vivo Targeted Drug Delivery and Controlled Release in B Cells Tumor Context,” Nanoscale 2(10):2230-2236; Ling, M. M., Wang, K. Y., and Chung, T.-S. 2010. “Highly Water-soluble Magnetic Nanoparticles as Novel Draw Solutes in Forward Osmosis for Water Reuse,” American Chemical Society. Industrial and Engineering Chemistry Research 49(12):5869-5876; Hu, F., MacRenaris, K. W., Waters, E. A., Liang, T., Schultz-Sikma, E. A., Eckermann, A. L., and Meade, T. J. 2009. “Ultrasmall, Water-soluble Magnetite Nanoparticles with High Relaxivity for Magnetic Resonance Imaging,” Journal of Physical Chemistry. C. Nanomaterials and Interfaces 113(49):20855-20860; Zablotskaya, A., Segal, I., Lukevics, E., Maiorov, M., Zablotsky, D., and 3 others 2009. “Water-soluble Magnetic Nanoparticles with Biologically Active Stabilizers,” Journal of Magnetism and Magnetic Materials 321(10):1428-1432 Wu, W., He, Q., and Jiang, C. 2008. “Magnetic Iron Oxide Nanoparticles: Synthesis and Surface Functionalization Strategies,” Nanoscale Research Letters 3(11):397-415; Sun, S. and Zeng, H. 2002. “Size-controlled Synthesis of Magnetite Nanoparticles,” Journal of the American Chemical Society 124(28):8204-8205).

In some cases, the ability to target one heart without affecting the other could stands in contrast to the conventional configuration where targeting thus is not possible. Because with the aid of an implanted automatic disorder response system a second smaller heart placed in the chest can provide a favorable outcome, the poor outcomes attributable to intolerance to the addition of a significantly undersized heart in a conventional heterotopic transplant can be avoided (see, for example, Bleasdale, R. A., Banner, N. R., Anyanwu, A. C., Mitchell, A. G., Khaghani, A., and Yacoub, M. H. 2002. “Determinants of Outcome after Heterotopic Heart Transplantation,” Journal of Heart and Lung Transplantation (New York, New York) 21(8):867-873; Kawaguchi, A. T., Gandjbakhch, I., Desruennes, M., Pavie, A., Bors, V., and 5 others named 1995, Op cit. above in section 2b(1)(b)(b3), entitled Neonatal and Infant Compound Vascular Bypass, or Sudden Switched- organ Transplantation of the Heart). The intravascular diversion servovalves allow the continuous adjustability in the proportional contribution to in- and outflow at any ostium or any combination of ostia provided by either heart. In a significant size mismatch, it is the apportionment of the venous return servovalves that adjust for the difference in sizes of the hearts.

Postprocedurally, the conventional heterotopic transplant is inaccessible to all but transcatheteric or transluminal treatments without open surgical reoperation or the discriminatory application of drugs. Nothing comparable to the degree of postprocedural diagnosis and responsive action, automated and acting instantly away from the clinic in a crisis offered by the switch technique in combination with an implanted automatic response system, pertains to the variants of conventional heterotopic technique, of which postoperative measures consist of systemic drugs and/or surgical reentry, invariably indiscriminate as regards all structures and tissues other than that intended. Indeed, no drug can be used or used in the most effective dose without the constraints imposed by the need to avoid injury elsewhere in the body. Indeed, conventional procedures are comparatively crude.

What must technically be allowed as the first heterotopic heart transplant was performed in October 1905 by Alexis Carrel and Charles Guthrie who removed the heart from a small dog and anastomosed it to vessels in the neck of a larger dog. Shortly after the operation, the graft heart was felt to pulsate. However, given that the beat generating conduction system is internal to the heart and semiautonomous, and the fact that no means to prevent rejection existed, the operation confirmed the sufficiency of the anastomoses and fact of graft perfusion but only as a ‘non-working model.’

Such did not represent functioning organ transplantation (Stolf, N. A. G. 2017. “History of Heart Transplantation: A Hard and Glorious Journey,” Brazilian Journal of Cardiovascular Surgery 32(5):423-427; Flecher, E., Fouquet, O., Ruggieri, V. G., Chabanne, C., Lelong, B., and Leguerrier, A. 2013, Op cit. above in this section; Sade, R. M. 2005. “Transplantation at 100 Years: Alexis Carrel, Pioneer Surgeon,” Annals of Thoracic Surgery 80(6):2415-2418; Novitzky, D., Cooper, D. K. C., and Barnard, C. N. 1983. “The Surgical Technique of Heterotopic Heart Transplantation,” Annals of Thoracic Surgery 36(4):476-482).

As with conventional heterotopic heart transplantation, double heart transplantation as described herein seeks to alleviate the shortage of replacement hearts by expanding the zone of marginal hearts made acceptable. It does this, however, not only with the superior periprocedural support of an implanted response system that instantly detects and responds to a change in status, but by sparing the ischemia and surgical trauma of conventional heterotopic transplantation, which to join the two, severely wounds both the native and donor hearts. Mechanical devices used to bridge to transplantation so far incapable of lifelong service and posing serious risks, a method to enable proceeding directly to transplantation without the need to wait for a good replacement heart would eliminate the need for a mechanical device.

Heterotopic heart transplantation responded to the realization that rather than to remove and replace the native heart, adding a second heart to assist that native in ventricular function would not result in total dependency upon an orthotopic transplant (which at the time, was almost certain to be rejected). Rather than an alternative model which anastomosed the added to the native heart to function as a left ventricular assist device, the configuration settled upon, the conventional configuration, or Barnard-Losman ‘biventricular model,’ was devised to pass the outflow from two separate right and left ventricles into aortae and pulmonary arteries merged by anastomosis.

While the native heart could continue to keep the recipient alive, undoing the surgery thereby inflicting additional trauma on an already impaired heart to extricate the added heart would not appear a Before the advent of cyclosporine, retaining the native heart served to protect the patient in the event that the graft heart was rejected (Newcomb, A. E., Esmore, D. S., Rosenfeldt, F. L., Richardson, M., and Marasco, S. F. 2004. “Heterotopic Heart Transplantation: An Expanding Role in the Twenty-first Century?,” Annals of Thoracic Surgery 78(4):1345-1351).

That given immunosuppressant drugs the odds for rejection are diminished, and the fact that the assistance lent by an added heart appears not to allow the native heart to recover to normal function, so that the practical impossibility of undoing the conventional, or Barnard-Losman configuration in order to remove the added heart and thereby alleviate the lifelong need for immunosuppressants is not a relevant for performing the procedure (Desruennes, M., Muneretto, C., Gandjbakhch, I., Kawaguchi, A., Pavie, A., 5 others named, et al. 1989. “Heterotopic Heart transplantation: Current Status in 1988,” Journal of Heart Transplantation 8(6):479-485).

One respect in which such conventional heterotopic transplantation appeared to expand the zone of usable hearts to include those extended criteria donors (marginal, less than ideal) was to allow the use of significantly smaller hearts contraindicated as orthotopic replacements for larger hearts, and while in some cases this is true, it became clear that overall, the configuration was size mismatch-sensitive in reducing survival over orthotopic transplantation of a properly sized heart (Flecher, E., Fouquet, O., Ruggieri, V. G., Chabanne, C., Lelong, B., and Leguerrier, A. 2013, Op cit.; Bleasdale, R. A., Banner, N. R., Anyanwu, A. C., Mitchell, A. G., Khaghani, A., and Yacoub, M. H. 2002, Op cit. above in this section; Kawaguchi, A. T., Gandjbakhch, I., Desruennes, M., Pavie, A., Bors, V., and 5 others, et al. 1995, Op cit. above in this section).

Another respect is where the prospective donor heart is leukocyte antigen mismatched (Godfrey, E. L., Kueht, M. L., Rana, A., and Frazier, O. H. 2019. “The Man with 2 Hearts: 25 Years from Heterotopic to Orthotopic Heart Transplantation,” Texas Heart Institute Journal 46(3):199-202). When both hearts working together cannot generate sufficient power of ejection even when assisted medically and with electrostimulation, the only long-term solution is an orthotopic transplantation of a competent heart, which can be accomplished using switch technique.

Interim bridging to transplantation can be accomplished with a ventricular or biventricular assist device or a total artificial heart. In retrospect, even with this conventional configuration, an implanted automatic disorder detection and response system as described in U.S. Pat. No. 11,759,186 could target a positive inotrope directly to the coronary arteries of either or both hearts, avoiding the numerous noncardiac side effects associated with these drugs, with a pacemaker placed to irregular heartbeat, the dilated heart could not be targeted for anticoagulation; instead warfarin or a heparinoid was dispersed systemically, leading to bleeding problems.

Here the warfarin (coumarin) or its derivative phenprocoumon, or 4-hydroxycoumarin derivative acenocoumarin (acenocoumarol), or a newer direct oral anticoagulant (fibrinolytic, thrombolytic, direct thrombin inhibitor) such as the clotting protein factor Xa inhibitors, or xabans, apixaban, betrixaban, dabigatran, edoxaban, rivaroxaban, and the injected or infused forms enoxaparin sodium and fondaparinux—or thrombolytic, or ‘clot buster,’ such as streptokinase, urokinase, or tissue plasminogen activator (alteplase, anistreplase, reteplse, or tenecteplase)—could be directly targeted to the coronary arteries of the native heart in a higher than system dose. With vitamin K as a reversal agent for warfarin, aminocaproic acid and tranexamic acid as fibrinolysis inhibitors, and andexanet alfa for the Xa inhibitors, the problem bleeding these agents may cause can be ameliorated. However, all of the above anticoagulants, thrombolytics, and reversal agents are disseminated systemically, which cannot compete with directly pipe-targeted administration for avoiding complications. Because administered thus anticoagulants and thrombolytics can be applied in the lowest dose effective, the more localized side effects that might arise can be suppressed by the reversal agents specified above where both the medication and its counteractant are kept from the rest of the body. The directly pipe-targeted delivery of any drug in liquid form into a ductus, usually a blood vessel is shown in FIGS. 16 and 21 of U.S. Pat. No. 11,759,186, entitled Ductus Side-entry Jackets and Prosthetic Disorder Response Systems. At the same time, drug targeting into a volume of tissue is through a nonjacketing side-entry connector such as shown in U.S. Pat. No. 11,013,858, FIG. 12A. These related documents specify several means for capturing any residue that would best be kept from entering the general circulation.

While the conventional, or Barnard-Losman configuration would still make possible the use of marginal hearts of like size as that native, the problem with undersized hearts would ordinarily disqualify the procedure, requiring instead the orthotopic transplantation of a like-sized competent heart, which if unavailable, would necessitate the use, if only to bridge to a transplant, of a ventricular or biventricular assist device or a total artificial heart.

Accordingly, an object here is to devise methods for rendering acceptable marginal hearts routinely rejected for transplantation (see, for example, Mancini, D., Goldstein, D., Taylor, S., Chen, L., Gass. A., DeLair, S., and Pinney, S. 2017. “Maximizing Donor Allocation: A Review of UNOS [United Network for Organ Sharing] Region 9 Donor Heart Turn-downs,” American Journal of Transplantation 17(12):3193-3198; Khush, K. K., Zaroff, J. G., Nguyen, J., Menza, R., and Goldstein, B. A. 2015. “National Decline in Donor Heart Utilization with Regional Variability: 1995- 2010,” American Journal of Transplantation 15(3):642-649; White, T. 2015. “Growing Number of Donor Hearts Rejected while Need for Transplants Rises, Study Finds,” Stanford Medicine News Center, Online at https://medstanford.edu/news/all-news/2015/02/growing-number-of-donor-hearts-rejected.html). Rejection and reperfusion injury looming in the best of circumstances, unaided, such hearts either lack the competency to be entrusted with the life of a patient or are rejected for defensive reasons.

Here methods are described that expand the zone of acceptability for transplantation of hearts that would otherwise be considered unacceptable. This comprises a fully implanted automatic disorder response system as described in U.S. Pat. No. 11,759,186, which able to target medication to any intracorporeal treatment site, can instantly sense the need for and provide medical support to an impaired native heart, administer the replacement of the native heart using the switch methods addressed herein and then support the new heart, or can transplant a second heart to assist an impaired native heart (but differently than in a conventional heterotopic transplant), or can replace the native heart with two hearts which together can dependably provide the left and right cardiac output required.

The immediate diagnostic and targeted drug delivery made possible by the same implanted automatic disorder response system used to execute the transplant should also improve outcomes where either or both donor and recipient are older, and do so even in single switch heart transplantation (see, for example, Chew. H. C., Macdonald, P. S., and Dhital, K. K. 2019. “The Donor Heart and Organ Perfusion Technology,” Journal of Thoracic Disease 11 (Supplement 6):S938-S945; Khush, K. K. 2018. “Donor Selection in the Modern Era,” Annals of Cardiothoracic Surgery (Hong Kong, China) 7(1):126-134; Dayoub, J. C., Cortese, F., Anžič, A., Grum, T., and de Magalhães, J. P. 2018. “The Effects of Donor Age on Organ Transplants: A Review and Implications for Aging Research,” Experimental Gerontology 110:230-240; Kittleson, M. M. 2018. “Recent Advances in Heart Transplantation,” F1000Research 7. pii: F1000 Faculty Rev-1008; Reich, H. J., Kobashigawa, J. A., Aintablian, T., Ramzy, D., Kittleson, M. M., and Esmailian, F. 2018. “Effects of Older Donor Age and Cold Ischemic Time on Long-term Outcomes of Heart Transplantation,” Texas Heart Institute Journal 45(1):17-22; Tryon, D., Hasaniya, N. W., Jabo, B., Razzouk, A. J., Bailey, L. L., and Rabkin, D. G. 2018. “Effect of Left Ventricular Dysfunction on Utilization of Donor Hearts,” Journal of Heart and Lung Transplantation (New York, New York) 37(3):349-357; Shudo, Y., Cohen, J. E., Lingala, B., He, H., and Woo, Y. J. 2018. “Impact of Donor Obesity on Outcomes after Orthotopic Heart Transplantation,” Journal of the American Heart Association (Oxford, England) 7(23):e010253; Tahir, H., Sachdev, S., Nguyen, L., Bardia, N., Omar, B., and Cohen, M. V. 2018. “Myocardial Stunning after Electrocution with Complete Reversibility within 24 Hours: Role of Repeat Transthoracic Echocardiograms in Potential Cardiac Transplant Donors,” Cardiology Research 9(4):268-272; White, C. W., Messer, S., Large, S. R., Conway, J., Kim, D. H., and 3 others 2018. “Transplantation of Hearts Donated after Circulatory Death,” Online, Frontiers in Cardiovascular Medicine 5:8; Hassanain, M., Simoneau, E., Doi, S. A., Aljiffry, M., Aloraini, A., Madkhali, A., and Metrakos, P. 2016. “Trends in Brain-dead Organ Donor Characteristics: A 13-year Analysis,” Canadian Journal of Surgery 59(3):154-160; Caplan, A. L. 2016. “Finding a Solution to the Organ Shortage,” Canadian Medical Association Journal pii: emaj. 151260; Girlanda, R. 2016. “Deceased Organ Donation for Transplantation: Challenges and Opportunities,” Online, World Journal of Transplantation (Pleasanton, California) 6(3):451-459; Guglin, M. 2015. “How to Increase the Utilization of Donor Hearts?,” Heart Failure Reviews 20(1):95-105; Sorabella, R. A., Guglielmetti, L., Kantor, A., Castillero, E., Takayama, H., and 4 others 2015. “Cardiac Donor Risk Factors Predictive of Short-term Heart Transplant Recipient Mortality: An Analysis of the United Network for Organ Sharing Database,” Transplantation Proceedings 47(10):2944-2951; Saidi. R. F. and Hejazii Kenari, S. K. 2014. “Challenges of Organ Shortage for Transplantation: Solutions and Opportunities,” International Journal of Organ Transplantation Medicine 5(3):87-96; Kilic, A., Emani, S., Sai-Sudhakar, C. B., Higgins, R. S., and Whitson, B. A. 2014. “Donor Selection in Heart Transplantation,” Journal of Thoracic Disease 6(8):1097-1104; Paul, J. J., Weber, D. J., Wang. I. W., Gracon, A. S., Hellman, Y. M., Hormuth, D. A., Wozniak, T. C., and Hashmi, Z. A. 2014. “Impact of Donor Age on Survival after Heart Transplantation: An Analysis of the United Network for Organ Sharing (UNOS) Registry,” Journal of Cardiac Surgery 29(5):723-728; Tani, L. Y., Shaddy, R. E., and Minich, L. L. 2003. “Spectrum of Left Ventricular Dysfunction in Potential Pediatric Heart Transplant Donors,” Journal of Heart and Lung Transplantation (New York, New York) 22(5):548-552; Jeevanandam, V., Furukawa, S., Prendergast, T. W., Todd, B. A., Eisen, H. J., and McClurken, J. B. 1996. “Standard Criteria for an Acceptable Donor Heart are Restricting Heart Transplantation,” Annals of Thoracic Surgery 62(5):1268-1275; Alexander, J. W. and Zola, J. C. 1996. “Expanding the Donor Pool: Use of Marginal Donors for Solid Organ Transplantation,” Clinical Transplantation 10(1 Part 1):1-19).

Exacerbating the shortage is the increasing incidence of obesity and the metabolic syndrome in the general population (Hassanain, M., Simoneau, E., Doi, S. A., Aljiffry, M., Aloraini, A., Madkhali, A., and Metrakos, P. 2016. “Trends in Brain-dead Organ Donor Characteristics: A 13-year Analysis,” Canadian Journal of Surgery 59(3):154-160), affecting a quarter of the population in the United States (Falkner, B. and Cossrow, N. D. 2014. “Prevalence of Metabolic Syndrome and Obesity-associated Hypertension in the Racial Ethnic Minorities of the United States,” Current Hypertension Reports 16(7):449; Beltran-Sanchez, H., Harhay, M. O., Harhay, M. M., and McElligott, S. 2013. “Prevalence and Trends of Metabolic Syndrome in the Adult U.S. Population, 1999-2010,” Journal of the American College of Cardiology 62(8):697-703).

When a sound replacement heart is not available, second auxiliary heart metered compound vascular bypass, or sudden switched-organ transplantation can be applied to a patient with end-stage congestive heart failure to obtain greater left ventricular ejection capability than either the native or added heart could supply on its own. The procedure is also applied to adding an accessory, of auxiliary, heart to that native in a ‘brain dead’ subject in preparation to transplant the paired hearts into a recipient with pronounced heart failure, for example, who may be close to death, whether an infant due to irreparable congenital malformation or an adult with end-stage heart failure. If the combined stroke volume of the paired hearts produces pulmonary hypertension, the excess pressure is relieved by tapping off a portion of flow into another large nearby vessel.

The routine implantation of an electronic synchronization device and the use of arrhythmia countering drugs in a conventional heart transplant remains unsettled (see, for example, Wdowczyk, J., Makowiec, D., Gruchala, M., Wejer, D., and Struzik, Z. R. 2018. “Dynamical Landscape of Heart Rhythm in Long-term Heart Transplant Recipients: A Way to Discern Erratic Rhythms,” Online, Frontiers in Physiology 9:274; Thajudeen, A., Stecker, E. C., Shehata, M., Patel, J., Wang, X., and 3 others 2012. “Arrhythmias after Heart Transplantation: Mechanisms and Management,” Journal of the American Heart Association (Oxford, England) 1(2):e001461 also cited above; McDowell, D. L. and Hauptman, P. J. 2009. “Implantable Defibrillators and Cardiac Resynchronization Therapy in Heart Transplant Recipients: Results of a National Survey,” Journal of Heart and Lung Transplantation (New York, New York) 28(8):847-850).

In the absence of practical experience, the number of variables is large enough that the subject can be no more than speculative; if only one continues to function normally without being significantly affected by a malfunction in the other, the net effect on the circulation is difficult to predict. In this circumstance, at least wearable devices for both the orthotopically and heterotopically placed hearts should be worn until it appears that the continued use thereof is not essential.

Upon detection of a ventricular fibrillation, for example, the accessory channels incorporated into the valve-jackets allow the implanted disorder response system to directly target each valve jacket and the vessel to which it is connected, selectively and differentially as sensed. Detection of a ventricular fibrillation is prescription-programmed to signal the need for cardiopulmonary resuscitation, release epinephrine immediately, and at about 4 minute intervals to either or both ventricles as appropriate, which if unsatisfactory or exacerbating by producing nausea, dizziness, or apprehension, is followed by the targeted release of amiodarone.

The question as to whether to place electronic resynchronization devices at the time of transplantation due to eventual difficulties should the need for retransplantation, infection, malfunction, or device manufacturer recall demand explantation so that the extraction of lead remnants becomes necessary can be answered in the affirmative. Wearable devices can be used until the need therefor is confirmed, and if it is, then implantable devices placed postoperatively after an interval long enough to allow the patient to recover, or if the need therefor is understood, placement can be at the time of transplantation. Using switch technique, no portion of the native heart itself remains, but the superior vena cava stump does, and may retain lead fragments which endothelialized and adhesion-bound, would cause injury if pulled out with force. At the same time, current guidelines are to remove electronic cardiac device leads implanted in the native heart prior to transplantation.

While not specifically addressed, to remove retained lead fragments in either a conventional ‘piggyback’ or switched-organed heterotopic transplantation of a second intact heart would certainly be recommended were these situations addressed (see, for example, Pettit, S., Orzalkiewicz, M., Nawaz, M. A., Lewis, C., Parameshwar, J., and Tsui, S. 2018. “Retained Pacemaker and Implantable Cardioverter-Defibrillator Components after Heart Transplantation are Common and May Lead to Adverse Events,” Europace 20(8):1312-1317; Holzhauser, L., Imamura, T., Nayak, H. M., Sarswat, N., Kim, G., and 9 others 2018. “Consequences of Retained Defibrillator and Pacemaker Leads after Heart Transplantation—An Underrecognized Problem,” Journal of Cardiac Failure 24(2):101-108).

Using the switch technique, if the native heart is to remain, the second or assist heart is transplanted intact using the metered switch technique described in the next section, then the vessels of the two are respectively joined by merging flow through the assist heart into that of the native heart through the mainlines of side-entry jackets with accessory channels to directly pipeline target drugs as necessary to the blood supply. If the native heart is so impaired that it would best be replaced and only two less than ideal replacement hearts are available, the native heart is replaced with the two less than ideal hearts. In a Level 1 procedure, the two donor hearts are transplanted in succession, the one heart removed from one deceased on somatic support and placed in the recipient, then the second replacement heart placed in the recipient.

This affords a preliminary induction of immune tolerance between the donor hearts. In a Level 2 procedure, the two have been placed in one of the deceased donors and then transplanted together into the recipient. This is the less desirable of the options, undertaken to reduce the time to transplantation where the recipient faces imminent death. While the donor hearts will have undergone a measure of tolerance induction, that accomplished will have been less than when the steps are less gradual, so that the process is three-way during transplantation of the two donor hearts into the recipient.

That is, if the native heart is far more impaired than either of two less than perfect hearts available, then the native heart is replaced using the metered switch technique, and the second heart is as well, the pulmonary arterial outflow merged along with the other like major vessels respectively as indicated. The second heart is treated as was the first, except that rather than having the expansion fabric mainlines and druglines disconnected and the native organ excised, the diversion servovalve connections between the first and second hearts are left in place.

The major vessels connected, there is no need to separately connect subsidiary vessels such as the coronary arteries and veins. Ideally, the recipient with end-stage heart disease is positioned with both donors parallel to one another and to the left side of the recipient. The two donor hearts can be transplanted in succession, but the procedural duration is reduced by having the two joined donor hearts joined to each other before the two are joined to and placed in the recipient.

The space needed to accommodate a second intact heart is more than that needed for a conventional heterotopic transplant, but in some cases, may not prove prohibitive as to necessitate an abdominal or infrarenal placement. However, if one heart is to be positioned orthotopically and the other heterotopically in the abdomen, then the expansion fabric lines and conduits are set o the correct length at the outset. A buildup of clot in the lines is prevented through the use of intrinsically clot repellent materials in their manufacture or a clot repellent internal surface coating and by an automatically released slow low molecular weight heparin or possibly the platelet blocker ticagrelor as a drip administered by the implant microcontroller.

The heparin is directly pipe-targeted and sensor monitored to maintain a total volume well below that essential to induce thrombocytopenia (see, for example, Alsalman, M. 2023. “A Case-report of Ticagrelor-induced Thrombocytopenia,” Online, International Medical Case Reports Journal (Auckland, New Zealand) 16:401-405: Nicolas, D. 2023. “Heparin-induced Thrombocytopenia,” Online, Treasure Island, Florida: StatPearls Publishing Co.; Sruthi, J. and Khanhar, P. B. 2022. “Thrombocytopenia,” Online, Treasure Island, Florida: StatPearls Publishing Co.; Acouthmany, S., Siano, A., and Bustillo, M. 2020. “HIT [Heparin Induced Thrombocytopenia]-Heparin Induced Thrombocytopenia Simulation Case, Online, Journal of Education and Teaching in Emergency Medicine (Orange, California) 6(1):S24-S45; Ahmed, I., Majeed. A., and Powell, R. 2007. “Heparin Induced Thrombocytopenia: Diagnosis and Management Update, Online, Postgraduate Medical Journal (London, England) 83(983):575-582).

While both the chest and abdomen are incised, the procedure is critically less traumatic than a conventional heart transplant on the hearts themselves, and the procedure has the numerous and fundamental advantages enumerated above in section 2 entitled Background of the Invention. a. General. Remote placement of a second heart is routine in clinical research (see, for example, Benke, K., Sayour, A. A., Mátyás, C., Agg. B., Nemeth, B. T., and 7 others 2017. Op cit., above in this section; additional references follow); however, the flow of blood through the remotely positioned heart is not by simple connection to the inferior vena cava and abdominal aorta as in an animal model but rather through the different vessels and chambers as is normal.

Heterotopic heart transplant models performed on animals solely for conducting immunological research are usually unsuitable both anatomically and physiologically for application in human patients (references pertaining to loading are cited in the following paragraph). In an abdominal positioning, the blood supply and drainage are not directly connected to the aorta and inferior vena cava respectively, but the vessels of the abdominally placed heart are confluent with the vessels of the orthotopically positioned heart.

More conventional methods, to include post-Barnard-Losman human modifications and experimental animal models are well documented, and unlike the compound vascular bypass metered switch technique, to join the two, all inflict considerable trauma on both the native and graft organs (see, for example, Westhofen, S., Jelinek, M., Dreher, L., Biermann, D., Martin, J., and 4 others 2019. “The Heterotopic Heart Transplantation in Mice as a Small Animal Model to Study Mechanical Unloading-Establishment of the Procedure, Perioperative Management and Postoperative Scoring,” Online, PLoS [Public Library of Science] One 14(4):e0214513; Schaefer, A., Schneeberger, Y., Schulz, S., Krasemann, S., Werner. T., and 12 others 2019. “Analysis of Fibrosis in Control or Pressure Overloaded Rat Hearts after Mechanical Unloading by Heterotopic Heart Transplantation,” (Nature Research) Scientific Reports 9(1):5710; Pokorný, M., Mrazova. I., Sochman, J., Melenovsky. V., Maly, J., and 9 others 2018. “Isovolumic Loading of the Failing Heart by Intraventricular Placement of a Spring Expander Attenuates Cardiac Atrophy after Heterotopic Heart Transplantation,” Bioscience Reports 38(3). pii: BSR20180371; Vaijyanath, P. 2017, Op cit. above in this section; Stolf. N. A. G. 2017. “History of Heart Transplantation: A Hard and Glorious Journey,” Braziian Journal of Cardiovascular Surgery 32(5):423-427; Holinski, S., Hausdorf, G., and Konertz, W. 2016, Op cit. above in section 2. Background of the Invention a. General; Lu. W., Zheng. J., Pan, X-D., Li, B., Zhang, J. W., Wang. L. F., and Sun, L. Z. 2015. “A New Simplified Volume-loaded Heterotopic Rabbit Heart Transplant Model with Improved Techniques and a Standard Operating Procedure,” Journal of Thoracic Disease 7(4):653-661; Jahanyar, J., Koerner, M. M., Ghodsizad, A., Loebe, M., and Noon, G. P. 2014. “Heterotopic Heart Transplantation: The United States Experience,” Heart Surgery Forum 17(3):E132-E140; Flecher, E., Fouquet, O., Ruggieri, V. G., Chabanne, C., Lelong. B., and Leguerrier, A. 2013, Op cit., this section; Didie, M., Biermann, D., Buchert, R., Hess, A., Wittkopper, K., and 7 others 2013. “Preservation of Left Ventricular Function and Morphology in Volume-loaded versus Volume-unloaded Heterotopic Heart Transplants,” American Journal of Physiology. Heart and Circulatory Physiology 305(4):H533-H541; Wen, P., Wang, X., Li, T., Zhang, B., Sun, X., and 6 others 2013. “Establishment of a Novel Volume-loaded Heterotopic Heart Transplantation Model in Rats,” Journal of Surgical Research 183(1):435-441; Plenter, R. J., Zamora, M. R., and Grazia, T. J. 2013. “Four Decades of Vascularized Heterotopic Cardiac Transplantation in the Mouse,” Journal of Investigative Surgery 26(4):223-228; Copeland, H., Kalra, N., Gustafson, M., Coehlo-Anderson, R., Friedman, M., and Copeland, J. G. 2011. “A Case of Heterotopic Heart Transplant as a “Biologic Left Ventricular Assist” in Restrictive Cardiomyopathy,” World Journal for Pediatric and Congenital Heart Surgery 2(4):637-640; Ma. Y. and Wang, G. 2011. “Comparison of 2 Heterotopic Heart Transplant Techniques in Rats: Cervical and Abdominal Heart,” Experimental and Clinical Transplantation 9(2):128-133; Shan, J., Huang, Y., Feng. L., Luo, L., Li, C., Ke, N., Zhang, C., and Li, Y. 2010. “A Modified Technique for Heterotopic Heart Transplantation in Rats,” Journal of Surgical Research 164(1):155- 161; Fiorelli, A. I., Coelho, G. H., Lima, J. L., Lourenço, D. D., Gutierres, P., and 4 others 2009, Op cit. above in section 2b(1)(b)(b3), entitled Neonatal and Infant Compound Vascular Bypass, or Sudden Switched-organ Transplantation of the Heart; Gordon, C. R., Matthews, M. S., Lefebvre, D. R., Strande, L. F., Marra, S. W., and 3 others 2007. “A New Modified Technique for Heterotopic Femoral Heart Transplantation in Rats,” Journal of Surgical Research 139(2):157-163; Smith, R. D. Jr., Radovancevic. B., Gregoric. I. D., Cohn. W. E., and Frazier, O. H. 2006. “Orthotopic Retransplantation in Heterotopic Transplant Recipients: 3 Case Reports,” Journal of Heart and Lung Transplantation (New York, New York) 25(2):245-247; Wang. D., Opelz, G., and Terness. P. 2006. “A Simplified Technique for Heart Transplantation in Rats: Abdominal Vessel Branch-sparing and Modified Venotomy,” Microsurgery 26(6):470-472; Newcomb. A. E., Esmore, D. S., Rosenfeldt, F. L., Richardson, M., and Marasco, S. F. 2004, Op cit.; Elefteriades, J., Lovoulos, C., Edwards, R., Tittle, S., Riley. T., and 3 others 2003. “Novel Technique for Isolated Accessory Right Heart Transplantation for Congenital Heart Disease,” Journal of Thoracic and Cardiovascular Surgery 125(6):1283-1290; Doenst, T., Schlensak, C., Kobba, J. L., and Beyersdorf, F. 2001. “A Technique of Heterotopic, Infrarenal Heart Transplantation with Double Anastomosis in Mice,” Journal of Heart and Lung Transplantation (New York, New York) 20(7):762-765; Asfour, B., Hare, J. M., Kohl, T., Baba, H. A., Kass. D. A., and 7 others 1999. “A Simple New Model of Physiologically Working Heterotopic Rat Heart Transplantation Provides Hemodynamic Performance Equivalent to that of an Orthotopic Heart,” Journal of Heart and Lung Transplantation (New York, New York) 18(10):927-936; Da Silva, J. P., Cascudo, M. M., Baumgratz, J. F., Vila, J. H., Wafae Filho, M., de Carvalho Neto, D. O., and Da Fonseca, L. 1994, Op cit., above in section 2a, entitled Background. General; Ohmi, M., Yokoyama, H., Nakame. T., Murata. S., Akimoto, H., Tabayashi, K., and Mohri. H. 1992. “Hemodynamic Performance in a Heterotopically Transplanted Dog Heart: Proposal of Techniques for Working Left Heart Model of Heterotopic (Abdominal) Heart Transplantation,” Journal of Heart and Lung Transplantation (New York, New York) 11(6):1147-1150; Bolman. R. M. 3rd. 1990. “Cardiac Transplantation: The Operative Technique,” Cardiovascular Clinics 20(2):133-145; Livi, U., Faggian, G., Chiominto, B., Mazzucco, A., Bortolotti, U., and Gallucci, V. 1990. “Heterotopic Heart Transplantation—A Means to Increase Donor Availability,” European Journal of Cardiothoracic Surgery 4(4):202-206; Reichenspumer. H., Hildebrandt, A., Boehm, D., Kaulbach, H. G., Willems, S., and 3 others 1989. “Heterotopic Heart Transplantation in 1988-Recent Selective Indications and Outcome,” Journal of Heart Transplantation 8(5):381-386; Konertz, W., Sheikhzadeh, A., Weyand, M., Friedl. A., and Bernhard, A. 1988, Op cit., above in section 2a, entitled Background of the Invention. General; Griffith, B. P., Kormos. R. L., and Hardesty, R. L. 1987. “Heterotopic Cardiac Transplantation: Current Status,” Journal of Cardiac Surgery 2(2):283-289; Cooper, D. K. C., Novitzky, D., Becerra, E., and Reichart, B. 1986. “Are There Indications for Heterotopic Heart Transplantation in 1986? A 2- to 11-year Follow-up of 49 Consecutive Patients Undergoing Heterotopic Heart Transplantation,” Thoracic and Cardiovascular Surgeon 34(5):300-304; Cooper, D. K. C3., 1984. “Orthotopic and Heterotopic Transplantation of the Heart: The Cape Town Experience,” Annals of the Royal College of Surgeons of England 66(4):228-234; Barnard, C. N., Barnard, M. S., Cooper, D. K. C. Curchio, C. A., Hassoulas, J., Novitsky, D., and Wolpowitz, A. 1981. “The Present Status of Heterotopic Cardiac Transplantation,” Journal of Thoracic and Cardiovascular Surgery 81(3):433-439; Barnard, C. N. and Losman, J. G. 1975. “Left Ventricular Bypass,” South African Medical Journal 49(9):303-312). Using switch transplantation, severe pulmonary vascular hypertension or the prospect thereof is best avoided by combined heart and lung transplantation.

It has been established that with any solid organ transplant, accompanying the graft organ with another, protolerogenic organ taken from the same donor increases tolerance induction for the primary graft organ. This is true whether an organ more resistant to immune tolerance induction, such as the heart, a lung, or both lungs, or more readily susceptible to tolerance induction, such as the kidneys and liver. Accordingly, when switch-transplanting a heart, lung, or lungs, additionally switch transplanting a kidney and/or part of the liver taken from the same donor on somatic support should boost tolerance induction. Other than to buttress immunological acceptance of the primary graft organ, the transplantation of a supernumerary organ is unique to the heart as a pump which must eject blood with sufficient force to perfuse the entire body.

A kidney not needed to replace a diseased native kidney is positioned heterotopically, abdominally or infrarenally. If due to the lack of a fully competent replacement heart, one severely impaired native heart is replaced with two less than ideal hearts, then a fourth kidney, one each taken from either heart donor, is switch transplanted heterotopically. When both hearts will not fit orthotopically, one of the hearts and two graft kidneys are switch transplanted heterotopically. Once the primary graft organ or organs are immunologically stable, if causing discomfort, either or both heterotopically positioned kidneys are removed.

Solid organ transplantation by means of the sudden or metered switch technique consists of diverting the blood supply and drainage between the native and the graft organ. A heart transplant, for example, switches the blood supply and drainage from the native to the graft heart, thus removing that native from the circulatory system of the recipient and replacing it with the graft. In a sudden compound vascular bypass, or sudden switched-organ transplantation, supply and drainage to each heart remains at the full complement of blood respective of the recipient and donor until the valves are fully extended or closed. Then the recipient heart is abruptly cut off, supply and drainage switched from the native to the graft organ, which then receives its fully complement of blood from the recipient.

Now part of the circulatory system of the recipient, the graft heart is harvested and positioned in the central compartment of the mediastinum of the recipient whose native heart has been removed. This eliminates many potential sources of problems encountered in conventional heart transplantation such as ischemia-reperfusion injury, however, it does so in an abrupt manner that precludes the exchange of blood between recipient and donor. It therefore eliminates the key advantage in the metered technique, which is to accomplish reciprocal cross-microchimerization concurrent with transfer of the donor organ into the recipient.

In a metered compound vascular bypass, or sudden switched-organ transplantation, both recipient and donor hearts receive a full complement of blood, but the relative proportion contributed by the recipient is gradually increased until the valves are fully extended, closing off reciprocal cross-circulation and flow through the donor heart. Both donor and recipient organs receive a full complement of blood, except the sum of recipient and donor blood have been blended so that a greater proportion in the total volume is that of the recipient. Switch technique affords significant expansion in the zone of hearts acceptable for.

When the heart to be replaced or supplemented is unpredictable, graft hearts should be graded as to residual competency with any preparatory treatment such as angioplasty for assisting native hearts of different sizes and competencies by replacement or supplementation, alone or as paired with a second replacement heart. Except in exigent circumstances, to protect against the provocation of a vasospasm, dysrhythmia, cerebral or myocardial infarction, or sudden cardiac arrest when graft hearts are handled, for example, the hearts should always be pretreated to eliminate plaques, and any other defects such as a post infarction left ventricular aneurysm repaired through an endoventricular circular patch plasty, or Dor procedure, for example (Dor, V., Donato, M., Sabatier, M., Montiglio, F. and Civaia, F. 2001. “Left Ventricular Reconstruction by Endoventricular Circular Patch Plasty Repair: A 17-year Experience,” Seminars in Thoracic and Cardiovascular Surgery 13(4):435-447). Additional references are provided above in section 2b(1)(b)(b4), entitled Metered Compound Vascular Bypass, or Switched-organ Transplantation in an Adult.

When time permits, such pretreatment is allowed to heal prior to transplantation; when not, and the efficacy of treatment is reasonably assured, the heart is transplanted, given an opportunity to stabilize, and then treated. When the native heart retains some competency, and especially when a sound replacement heart cannot be found, a second heart, usually positioned heterotopically in the abdomen or infrarenally, is added to that native. This is accomplished as is a one-for-one transplant as just described, but rather than to fully extend the diversion chutes closing the valves of both native and donor hearts to divert all blood to the donor heart and cutting off the supply of blood to the native heart, the valves are moved to the half-way point. The native heart is therefore retained in at the same time that the graft heart has been added into the circulatory system of the recipient, with the volume of blood to each halved.

In May of 1952, Andreasen and Watson discovered that if the venae cavae were tied off, the azygos vein—a collateral that continues to deliver blood into the heart should the venae cavae become obstructed—would still sustain a sufficient venous return volume to maintain normal circulation without the inducement of a dysrhythmia or postoperative adverse cognitive consequence for at least a half hour (Andreasen, A. T. and Watson, F. 1952. “Experimental Cardiac Surgery,” British Journal of Surgery 391(158):548-551, available online at https://doi.org/10.1002/bjs.18003915819 and https://Onlinelibrary.wiley.com/doi/abs/10.1002/bis.18003915819). Cohen and Lillehei then engaged in a series of experiments to determine whether this volume of return—about ten percent of that normal-would allow them to perform intracardiac surgery.

They found that while a ninety percent reduction in venous return did not provide a completely bloodless surgical field, the chambers were sufficiently visible to allow interventricular septal defects, mitral annuloplasty, and even the more complex malformations involving, for example, the trilogy or the tetralogy of Fallot or the Eisenmenger complex to be repaired (see, for example, Gould, S. E. 1953. Pathology of the Heart, Springfield, Illinois: Charles C. Thomas; Lev, M. 1953. Diagnosis of Congenitally Malformed Hearts, Springfield, Illinois: Charles C. Thomas). With two hearts, venous return to each heart at half that normal is five times greater than venous return through the azygos vein. This indicates that with size-matched hearts, hence, the venae cavae servovalves of the native heart static at the halfway point, the risk of inducing a ventricular fibrillation which could lead to a sudden arrest is low over the initial period.

Following clarification as to the sufficiency of azygos flow to sustain the heart during intracardiac surgery, cross-circulation continued in use until cardiopulmonary machines-which were not limited to small patients, mostly children, due to the blood supply capacity of the donor, and posed no risk of brain damage to the donor were air bubbles to enter the lines, became sufficiently dependable (see for example, Department of Surgery, University of Minnesota, Cardiothoracic Staff 2019. “The Cross-Circulated Heart: Azygos Flow Principle” Online at http://gallery.lib.umn.edu/exhibits/show/openheart/closed; van Vuuren, T. M., Doganci, S., Toonder, I. M., Graaf, R., and Wittens, C. H. 2019. “Venous Stent Patency May Be Affected by Collateral Vein Lumen Size,” Phlebology 34(1):32-39; Mandato, Y., Pecoraro, C., Gagliardi, G., and Tecame, M. 2019. “Azygos and Hemiazygos Continuation: An Occasional Finding in Emergency Department,” Radiology Case Reports 14(9):1063-1068; Allen, C. J., Freeman, T., Perera, W., Padley, S. P., and Hind, M. 2018. “Thoracic Park: Cardiac MRI Reveals Massive Thoracic Varices as Consequence of Inferior Vena Cava Ligation,” European Heart Journal Cardiovascular Imaging 19(3):359-360; Cheung, C. K. M., Law, M. F., Wong, K. T., Tam, M. T. K., and Chow, K. M. 2018. “Massive Pulmonary Embolism in a Patient with Polysplenia Syndrome and Interrupted Inferior Vena Cava with Azygos Continuation,” Archives of Medical Science 14(1):251-253; Pantin, E. J., Naftalovich, R., and Denny, J. 2016. “Echocardiographic Identification of an Interrupted Inferior Vena Cava with Dilated Azygos Vein during Coronary Artery Bypass Graft Surgery,” Anesthesia and Analgesia 2016 122(2):358-360; Piciucchi, S., Barone, D., Sanna, S., Dubini, A., Goodman, L. R., and 6 others 2014. “The Azygos Vein Pathway: An Overview from Anatomical Variations to Pathological Changes,” Insights into Imaging 5(5):619-628; Paoletti, F., Pellegrino, V., Antonelli, M., Ripani, U., Mosca, S., Duri, D., and Galzerano, A. 2009. “Compensatory Dilatation of the Azygos Venous System Secondary To Superior Vena Cava Occlusion” Journal of Radiology Case Reports 3(12):49-55; Cohen M. and Lillehei, C. W. 1954. “A Quantitative Study of the Azygos Factor during Vena Caval Occlusion in the Dog,” Surgery, Gynecology, and Obstetrics 98(2):225-232; Fleischner, F. G. and Udis, S. W. 1952. “Dilatation of the Azygos Vein; a Roentgen Sign of Venous Engorgement,” American Journal of Roentgenology, Radium Therapy, and Nuclear Medicine 67(4):569-575).

In a double heart transplant, only the native heart retains the azygos for venous return. However, the caval occlusive contingencies the azygos stands by to compensate for are eliminated because the synthetic lines used to move blood are not susceptible to and therefore eliminate potential causes for caval obstruction. The need for the azygos as an emergency venous collateral is therefore eliminated for the native and well as for the added heart.

The ‘azygos factor’ thus diminishes concern that double intracardiac hypovolemia would likely trigger a dysrhythmia in both hearts and hypotension in the recipient. While the azygos factor had to be relied upon for only so long as the intracardiac operation continued—for most about ninety minutes—it may be that venous return reduced to half, not just one tenth, continued over the long-term might eventually impair either or both hearts. It warrants emphasis that transplant hearts often require adjuvant support over the period needed to adapt to the new milieu. In a double heart transplant, both hearts gradually adapt to the novel condition, so that supportive measures are likely to prove essential for a short period, after which these can be terminated or removed.

This is significant, since the cost of pacemaker/cardioverter defibrillators is high; the temporary need therefore means that these can be retrieved for use in another patient. Techniques have been devised to substantially eliminate the trauma previously experienced when leads were removed (references cited above in this section). When the hearts are alike in size, the movement would typically be from complete extension of the heart in the chest to the heterotopically positioned heart, thus intermittently interrupting the reduction in flow to the latter of only half the normal volume of blood. The volume of blood is greater than that delivered through the azygos vein when compensation-dilated during caval obstruction

If concern over such a complication persists, measures to avert such a contingency are straightforward. Although not likely to be necessary, an artificial ‘azygos’ for the donor heart can be created by shunting a greater volume of venous blood than would be provided by a natural azygos from a neighboring larger vein, by means shown in U.S. Pat. No. 11,759,186. Another measure is the temporary placement of pacemaker/cardioverter defibrillators on each heart, which can not only maintain and reinstate normal rhythm, if necessary, but serve as sensors to the microcontroller or microprocessor to complement the action of the cardioverter defibrillators by initiating diversion valve adjustments or more frequent toggling and/or target medication to either or both hearts until sufficient time has passed for both hearts to adapt to the new conditions.

While placement of the second heart in the chest is preferred, an advantage in an anatomically inferior location for the second heart is that it allows the use of wearable cardioverter defibrillators on each heart. Nevertheless, methods addressed above in section 2b(1) have been developed to remove transvenous leads without significant injury, so that the temporary use and retrieval of implantable cardioverter defibrillators once confirmed to no longer be needed makes positioning the second heart beside that native or earlier transplanted in the chest to be preferred. Unless there is reason to retain these, the costly devices are then explanted for reuse using current best practice guidelines as cited above in this section to remove the leads.

That is, the activation of either or both cardioverter defibrillators signals the microcontroller or processor to initiate toggling, whereby both hearts receive the normal volume of venous return blood in alteration quickly enough to approximate normal return. For like sized hearts, this is accomplished by setting the venous return valves of the native heart to the midpoint, proportionally adjusted for hearts of different size. To provide a volume of blood to both hearts close enough to that normal, the diversion valves are set to reciprocate about this middle or half-way point in diversion chute extension adjusted as necessary, thus alternately sending the full volume of blood to both hearts frequently enough to avoid complications associated with cardiac hypovolemia.

At least temporarily, pacemaker/cardioverter-defibrillators can be placed to monitor both hearts. If necessary, these can be left in place until the hearts have adapted, first, sufficiently to perform well under conditions of reciprocal valving, then well enough to dispense with valve reciprocation. While metered switch double heart transplantation continues, a menacing dysrhythmia in either or both hearts can be prevented by gradual adjustment among the valves or by toggling that is, by initiating reciprocating extension and retraction of the native venous return valves at an adjustable lower rate and displacement to either side of the setpoints established on the basis of the relative sizes and condition of each of the hearts involved.

Toggling thus makes it possible to switch the valves of the primary heart from partial to full flow quickly enough that an intracardiac hypovolemia sufficient to induce a dysrhythmia should be avoidable. In this way, a volume and flow approximating that normal can, without meaningful interruption, be divided between two hearts. Should it become necessary, later removal or intracardiac surgery of the added heart is without the need for cross-clamping, the diversion chutes of the primary heart venae cavae, that heart positioned in the chest, fully retracted, thus cutting off caval venous retum flow to the secondary heart, effectively isolating the two.

If a repair rather than explantation is necessary, sufficient blood to sustain heart function is provided through a shunt created with a basic side-entry jacket from the closest suitable vein to deliver this amount, as shown in U.S. Pat. No. 11,759,186, entitled Ductus Side-entry Jackets and Prosthetic Disorder Response Systems. The likelihood is that only two servovalves-those of the aorta and main pulmonary artery, or trunk, of the primary, or lead heart, meaning that native or that orthotopically transplanted earlier, will need to be toggled. If the right and left pulmonary arteries are toggled separately, the others passive with diversion chutes fully extended, hence, drawing no power and allowing dispensing with the need for one or more paracorporeal batteries or battery packs, then three servovalves may be toggled.

These set the volume of blood that continues to flow through all of the other valves, which remain fully open, that is, with diversion chutes fully extended. Synchronized to the pulse, multiple, or a subdivision thereof, toggling, unlike implantable, or ambulatory, ventricular assist devices, supports pulsatile rather than continuous flow, and does so without right-left ventricular bias or the need for systemic anticoagulation.

Where some mechanical assist devices and total artificial hearts remain too large for application in small children, this does not apply to single or double heart transplantation as herein described, which can be applied to neonates. The implanted automatic disorder response system can be used in conjunction with conventional cardiological treatment, assist devices, and total artificial hearts; however, these cannot approximate the freedom of movement of the means described. Nonambulatory devices such as intra-aortic balloon pumps, assist devices, and artificial hearts for use in a clinic where the device can be plugged into an extracorporeal power source and connection to a control console prohibit freedom of movement. The addition to a failing heart of a second assist heart with automatic support by an implanted response system promises advantages over mechanical devices such as ventricular assist devices and total artificial hearts.

Design improvements in these devices notwithstanding, the advantages of the means described herein, especially a compound vascular bypass heterotopic double heart transplant over alternative approaches include 1. Negligible power consumption, 2. Almost entirely organic rather than synthetic, fewer components not recognized as integral to the body and protected by the innate immune system, 3. Little susceptibility to thrombosis, 4. Smaller size and weight, hence making possible a less impeded life 5. Automatically and directly pipe-targeted medicinal support to the innate immune system rather than the need for example, for exogenously obtained systemic anticoagulation, 6. Healing seals joints that would otherwise risk bleeding problems necessitating follow-up surgery and blood transfusions, and. 7. Increased bodily integrity means more comprehensive coverage by the innate immune system to reduce concomitant risks such as viral infections.

Less clear advantages probably include 8. An inherent capability of the graft organ to participate in the generation of a normal body temperature, and 9. A reduction in the number of critical moving parts which would not be automatically corrected if obstructed, for example (see, for example, Vaidya, Y., and Dhamoon, S. 2020. Op cit., section 2a: Wadowski, P. P., Steinlechner, B., Zimpfer. D., Schloglhofer, T., Schima. H., and 8 others 2019. “Functional Capillary Impairment in Patients with Ventricular Assist Devices,” (Nature Research) Scientific Reports 9(1):5909: Sidhu, K., Lam, P. H., and Mehra. M. R. 2019. “Evolving Trends in Mechanical Circulatory Support: Clinical Development of a Fully Magnetically Levitated Durable Ventricular Assist Device,” Trends in Cardiovascular Medicine pii: S1050-1738(19) 30077-5; Bansal, A., Uriel, N., Colombo, P. C., Narisetty, K., Long. J. W., and 13 others 2019. “Effects of a Fully Magnetically Levitated Centrifugal-flow or Axial-flow Left Ventricular Assist Device on von Willebrand Factor: A Prospective Multicenter Clinical Trial,” Journal of Heart and Lung Transplantation (New York, New York) pii: S1053-2498(19) 31508-6; Klaeske, K., Dieterlen, M. T., Scholz, U., Lehmann, S., Garbade, J, and 4 others 2019. “Acquired von Willebrand Factor Deficiency is Reduced in HeartMate 3 Patients,” European Journal of Cardiothoracic Surgery pii: ezz045; Peng, D. M., Koehl, D. A., Cantor, R. S., McMillan, K. N., Barnes, A. P., and 7 others 2019. “Outcomes of Children with Congenital Heart Disease Implanted with Ventricular Assist Devices: An Analysis of the Pediatric Interagency Registry for Mechanical Circulatory Support (Pedimacs),” Journal of Heart and Lung Transplantation (New York, New York) 38(4):420-430; Schweiger, M., Lorts, A., and Conway, J. 2018. “Mechanical Circulatory Support Challenges in Pediatric and (Adult) Congenital Heart Disease,” Current Opinion in Organ Transplantation 23(3):301-307; Gallo, M., Trivedi, J. R., Sobieski, M. A., Massey, T. H., Cheng, A., and Slaughter, M. S. 2017. “Surgical Technique for Ventricular Device Exchange: From HeartMate II to HVAD,” American Society for Artificial Internal Organs Journal 63(3):364-366; Burki. S. and Adachi, I. 2017. “Pediatric Ventricular Assist Devices: Current Challenges and Future Prospects,” Vascular Health and Risk Management 13:177-185; Chopski, S. G., Moskowitz, W. B., Stevens, R. M., and Throckmorton, A. L. 2017. “Mechanical Circulatory Support Devices for Pediatric Patients with Congenital Heart Disease,” Artificial Organs 41(1):E1-E14; Capoccia, M. 2016. “Mechanical Circulatory Support for Advanced Heart Failure: Are We about to Witness a New “Gold Standard”?,” Journal of Cardiovascular Development and Disease 3(4). pii: E35: Yost. G., Joseph, C. R., Royston, T., Tatooles. A., and Bhat, G. 2016. “Heat Generation in Axial and Centrifugal Flow Left Ventricular Assist Devices,” American Society for Artificial Internal Organs Journal 62(6):684-689; Rosenthal, D. N., Almond, C. S., Jaquiss. R. D., Peyton, C. E., Auerbach, S. R., and 9 others 2016. “Adverse Events in Children Implanted with Ventricular Assist Devices in the United States: Data from the Pediatric Interagency Registry for Mechanical Circulatory Support (PediMACS),” Journal of Heart and Lung Transplantation (New York, New York) 35(5):569-577; Pagani, F. D., Long, J. W., Dembitsky, W. P., Joyce. L. D., and Miller, L. W. 2006. “Improved Mechanical Reliability of the HeartMate XVE Left Ventricular Assist System,” Annals of Thoracic Surgery 82(4):1413-1418). Directly targeted immunosuppressant and anticoagulant agents are relatively minute in dose. Other advantages are greater durability, freedom from complications, and he elimination of externally worn battery packs and belt-worn control units, for example.

Providing reduced dimensions and improved mechanical dependability, continuous flow ventricular assist devices supplanted pulsatile models; however, these come with problems related to the damage done to several blood constituents associated with continuous flow (see, for example, Healy, A. H., Mckellar, S. H., Drakos, S. G., Koliopoulou, A., Stehlik, J., and Selzman, C. H. 2016. Physiologic Effects of Continuous-flow Left Ventricular Assist Devices,” Journal of Surgical Research 202(2):363-371; Garcia, S., Kandar, F., Boyle, A., Colvin-Adams, M., Lliao, K., Joyce, L., and John, R. 2008. “Effects of Pulsatile- and Continuous-flow Left Ventricular Assist Devices on Left Ventricular Unloading,” Journal of Heart and Lung Transplantation (New York, New York) 27(3):261-267). Left ventricular devices become more injurious over time; however, that time may be considerable, making the value of these as bridging devices to transplantation irrefutable.

Whether pulsatile or continuous flow, left ventricular assist devices induce right ventricular failure, over time ventricular atrophy (see, for example, Baxter, R. D., Tecson, K. M., Still, S., Collier, J. D. G., Felius, J., and 3 others 2019. “Predictors and Impact of Right Heart Failure Severity following Left Ventricular Assist Device Implantation,” Journal of Thoracic Disease 11 (Supplement 6):S864-S870; Pokomý, M., Mrázová, I., Kubátová, H., Pitha, J., Malý, J., and 9 others 2019. “Intraventricular Placement of a Spring Expander Does Not Attenuate Cardiac Atrophy of the Healthy Heart Induced by Unloading via Heterotopic Heart Transplantation,” Physiological Research [in press]; Pokorný, M., Mrazova, I., Sochman, J., Melenovsky, V., Maly, J., and 9 others 2018. “Isovolumic Loading of the Failing Heart by Intraventricular Placement of a Spring Expander Attenuates Cardiac Atrophy after Heterotopic Heart Transplantation,” Bioscience Reports 38(3). pii: BSR20180371: Puhlman, M. 2012. “Continuous-flow Left Ventricular Assist Device and the Right Ventricle,” American Association of Critical Care Nurses Advanced Critical Care 23(1):86-90; Kormos. R. L., Teuteberg, J. J., Pagani, F. D., Russell, S. D., John, R., 6 others 2010. “Right Ventricular Failure in Patients with the HeartMate II Continuous-flow Left Ventricular Assist Device: Incidence, Risk Factors, and Effect on Outcomes,” Journal of Thoracic and Cardiovascular Surgery 139(5):1316-1324; Patel, N. D., Weiss, E. S., Schaffer, J., Ullrich, S. L., Rivard, D. C., and 3 others 2008. “Right Heart Dysfunction after Left Ventricular Assist Device Implantation: A Comparison of the Pulsatile HeartMate I and Axial-flow HeartMate II Devices,” Annals o Thoracic Surgery 86(3):832-840).

Patients with cardiorenal syndrome secondary to heart failure with chronic renal disease do not experience renal recovery over the long-term and sometimes even lose kidney function (see, for example, Walther, C. P., Winkelmayer, W. C., Niu, J., Cheemna, F. H., Nair, A. P., and 4 others 2019. “Acute Kidney Injury with Ventricular Assist Device Placement: National Estimates of Trends and Outcomes,” American Journal of Kidney Diseases pii: S0272-6386(19) 30704-30708; Inampudi. C., Alvarez, P., Asleh, R., and Briasoulis, A. 2018. “Therapeutic Approach to Patients with Heart Failure with Reduced Ejection Fraction and End-stage Renal Disease,” Current Cardiology Reviews 14(1):60-66; Ross, D. W., Stevens, G. R., Wanchoo. R., Majure, D. T., Jauhar, S., and 3 others 2018. “Left Ventricular Assist Devices and the Kidney,” Clinical Journal of the American Society of Nephrology 13(2):348-355; Walther. C. P., Niu, J., Winkelmayer, W. C., Cheema, F. H., Nair, A. P., and 4 others 2018. “Implantable Ventricular Assist Device Use and Outcomes in People with End-stage Renal Disease,” Journal of the American Heart Association (Oxford, England) 7(14). pii: e008664; Bansal, N., Hailpern, S. M., Katz, R., Hall, Y. N., Kurella Tamura, M., Kreuter, W., and O'Hare, A. M. 2018. “Outcomes Associated with Left Ventricular Assist Devices among Recipients with and without End-stage Renal Disease,” Journal of the American Medical Association Internal Medicine 178(2):204-209).

Continuous flow is considered a drawback in Fontan circulation, and produced by ventricular assist devices and artificial hearts, perhaps due to the amount of anticoagulant required, is implicated in greater nonsurgical gastrointestinal bleeding than pulsatile machines, is considered inimical to cardiovascular physiology, and has been found to result in an increased incidence of stroke (see, for example. Cornwell, W. K. 3rd, Ambardekar, A. V., Tran, T., Pal. J. D., Cava. L., and 4 others 2019. “Stroke Incidence and Impact of Continuous-flow Left Ventricular Assist Devices on Cerebrovascular Physiology,” Stroke 50(2):542-548; Camicelli, A. P., Thakkar, A., Deicicchi, D. J., Storm, A. C., Rimsans, J., and 4 others 2019. “Resource Utilization and Hospital Readmission Associated with Gastrointestinal Bleeding in Patients with Continuous-flow Left Ventricular Assist Devices,” Journal of Thrombosis and Thrombolysis 47(3):375-383; Kataria. R. and Jorde, U. P. 2019. “Gastrointestinal Bleeding during Continuous-flow Left Ventricular Assist Device Support: State of the Field,” Cardiology in Review 27(1):8-13; Lampert, B. C. 2018. “Perioperative Management of the Right and Left Ventricles,” Cardiology Clinics 36(4):495-506; Welden, C. V., Truss, W., McGwin, G., Weber, F., and Peter. S. 2018. “Clinical Predictors for Repeat Hospitalizations in Left Ventricular Assist Device (LVAD) Patients with Gastrointestinal Bleeding,” Gastroenterology Research 11(2):100-105; Axelrad, J. E., Pinsino. A., Trinh, P. N., Thanataveerat. A., Brooks, C., and 15 others 2018. “Limited Usefulness of Endoscopic Evaluation in Patients with Continuous-flow Left Ventricular Assist Devices and Gastrointestinal Bleeding,” Journal of Heart and Lung Transplantation (New York, New York) 37(6):723-732; Al-Bawardy. B., Schettle, S. D., Gorospe, E., Kee Song, L. M. W., Pereira, N. L., and 8 others 2018. “Small Bowel Bleeding in Patients with Left Ventricular Assist Device: Outcomes of Conservative Therapy versus Balloon-assisted Enteroscopy,” Annals of Gastroenterology 31(6): 692-697; Kim, J. H., Brophy, D. F., and Shah, K. B. 2018. “Continuous-flow Left Ventricular Assist Device-related Gastrointestinal Bleeding,” Cardiology Clinics 36(4):519-529; Flint, K. M., Spertus, J. A., Tang, F., Jones, P., Fendler, T. J., and Allen, L. A. 2017. “Association of Global and Disease-specific Health Status with Outcomes following Continuous-flow Left Ventricular Assist Device Implantation,” Online, BioMed Central Cardiovascular Disorders 17(1):78; Li, F., Hinton, A., Chen, A., Mehta, N. K., Eldika, S., and 4 others 2017. “Left Ventricular Assist Devices Impact Hospital Resource Utilization without Affecting Patient Mortality in Gastrointestinal Bleeding,” Digestive Diseases and Sciences 62(1):150-160; Wever-Pinzon, O., Selzman, C. H., Drakos, S. G., Saidi, A., Stoddard, G. J., and 8 others 2013. “Pulsatility and the Risk of Nonsurgical Bleeding in Patients Supported with the Continuous-flow Left Ventricular Assist Device HeartMate II,” Circulation. Heart Failure 6(3):517-526).

Eliminating the need for body-worn, or paracorporeal, components, such as a power pack, system controller, and driver, alleviates the patient of impedimenta of a ventricular assist device or total artificial heart, which must be worn at all times. The use of mechanical cardiac devices often imperative, each of these comprises numerous parts upon each of which the life of the patient depends. Depending upon the design, a total artificial heart either replaces the native heart or requires the replacement of both ventricles as is also the case with a conventional orthotopic heart transplant, and both impose considerable trauma on the retained portions of the native heart.

Advancement in 2001 to a total artificial heart allowed the controller and battery to be implanted, leaving only the transdermal recharging components extracorporeal (see, for example, Ryan, T. D., Jefferies, J. L., Zafar, F., Lorts, A., and Morales, D. L. 2015. “The Evolving Role of the Total Artificial Heart in the Management of End-stage Congenital Heart Disease and Adolescents,” American Society for Artificial Internal Organs Journal 61(1):8-14; Gerosa, G., Scuri, S., lop, L., and Torregrossa, G. 2014. “Present and Future Perspectives on Total Artificial Hearts,” Annals of Cardiothoracic Surgery (Hong Kong, China) 36):595-602; Emergency Care Research Institute 2002. “AbioCor Totally Implantable Artificial Heart. How Will It Impact Hospitals?,” Health Devices 31(9):332-341).

However, the considerably greater power requirement than that required to power implanted vascular servovalves means that the battery must be larger and the recharging interval is far longer. Moreover, the risks of problem bleeding and clotting both mid- and postprocedurally remain considerable. In contrast, a single or double compound vascular bypass heart transplant requires no power source and leaves both native and donor hearts completely intact without incision.

The need to constantly wear a power pack or remain in the vicinity of a transdermal energy transfer source imposed by mechanical assist devices and total artificial hearts much degrades the freedom of movement and therewith, the quality of life, of these patients. A double heart transplant as addressed above in section 2b(1)(b)(b5), entitled Heterotopic Double Heart Metered Compound Vascular Bypass, or Switched-organ Transplantation [and so on], can serve as an organic ventricular assist device without the hardware and impediment of wearing a power pack and having to keep it charged at all times. Equally important, the complication encountered with continuous flow devices are avoided. Moreover, even though their life is at stake, most of the people in the world cannot afford such costly apparatus.

The power consumption demands of left ventricular and biventricular assist devices and artificial hearts are considerable enough to represent a constant nuisance to encourage avoiding these in favor of a replacement or assist allograft heart which imposes no such demand. However, throughout the developed world, this is thwarted by the shortage of available allografts. Two other problems with mechanical devices are “ . . . infections caused by percutaneous drivelines and thrombotic events associated with the use of blood-contacting surfaces,” (Han and Trumble 2019, cited below). A key inducement in favor of a metered compound vascular bypass double heart heterotopic transplant is that a less than perfect assist allograft heart is less scarce and can prove adequate to eliminate the need for an exogenous source of power.

Total implantability of mechanical devices primarily depends upon developing means for preventing 1. Percutaneous driveline infection, 2. Materials that thwart the accretion of clot, 3. The need for an extracorporeal source of power, such as through transcutaneous energy transfer, and 4. The radiation of heat to neighboring tissue which causes the patient discomfort. That these deficiencies pertain to assist devices and totally artificial hearts but to real hearts not in the least shows the preferability of heart transplantation whenever a suitable replacement heart is available.

On a worldwide basis, these devices are often unaffordable and without the local technical expertise their successful implantation demands. These factors notwithstanding, a balanced appraisal demands that an allotransplant poses the risk of foreign body adverse tissue reactions and rejection in addition to the complications cited by Pasha, et al. 2022 below, and infection is a risk in every surgical procedure.

Moreover, the means specified above in section 2a, entitled Background of the Invention. General would substantially increase the pool of usable hearts (see also, for example, Tu, J., Xu, L., Li, F., and Dong, N. 2024. “Developments and Challenges in Durable Ventricular Assist Device Technology: A Comprehensive Review with a Focus on Advancements in China,” Online, MDPI [Multidisciplinary Digital Publishing Institute] Journal of Cardiovascular Development and Disease (Basel, Switzerland) 11(1):29; Andriollo, M, Fanton, E., and Tortella, A. 2023. “A Review of Innovative Electromagnetic Technologies for a Totally Artificial Heart,” Online, MDPI [Multidisciplinary Digital Publishing Institute] Applied Sciences Basel, Switzerland) 13(3):1870; Karim, M. L., Bosnjak, A. M., Mclaughlin, J., Crawford, P., McEneaney, D., and Escalona, O. J. 2022. “Transcutaneous Pulsed R F Energy Transfer Mitigates Tissue Heating in High Power Demand Implanted Device Applications: In Vivo and In Silico Models Results,” Online, MDPI [Multidisciplinary Digital Publishing Institute] Sensors (Basel, Switzerland) 22(20):7775; Pasha, A. K., Lee, J. Z., Desai, H., Hashemzadeh, M., and Movahed, M. R. 2022. “In-hospital Complications Associated with Total Artificial Heart Implantation in the United States between 2004 to 2011,” Online, American Journal of Cardiovascular Disease (Madison, Wisconsin) 12(5):278-282; Joyce, L. D. and Joyce, D. L. 2020. “Total Artificial Heart: Neurological Complications,” Online, Annals of Cardiothoracic Surgery (Hong Kong, China) 9(2):121-123; Senage, T., David, C. H., Nanjaiah, P., and Roussel, J. C. 2020. “Total Artificial Heart: Patient Selection and Risk Factors,” Online, Annals of Cardiothoracic Surgery (Hong Kong, China) 9(2):118-120; Han, J. and Trumble, D. R. 2019. “Cardiac Assist Devices: Early Concepts, Current Technologies, and Future Innovations,” Online, MDPI [Multidisciplinary Digital Publishing Institute] Bioengineering (Basel, Switzerland) 6(1):18; Melton, N., Soleimani, B., and Dowling, R. 2019. “Current Role of the Total Artificial Heart in the Management of Advanced Heart Failure,” Current Cardiology Reports (Philadelphia, Pennsylvania) 21(11):142; Pathak, V., Donovan, C., and Malhotra, R. 2017. “Airway Complications of Total Artificial Heart,” Online, Indian Journal of Critical Care Medicine (New Delhi) 21(2):94-95; Goerlich, C. E., Frazier, O. H., and Cohn, W. E. 2016. “Previous Challenges and Current Progress—The Use of Total Artificial Hearts in Patients with End-stage Heart Failure,” Online, Expert Review in Cardiovascular Therapy (Abingdon, Oxford, England) 14(10):1095-1098; Cohn, W. E., Timms, D. L., and Frazier, O. H. 2015. “Total Artificial Hearts: Past, Present, and Future,” Nature Reviews. Cardiology (London, England) 12(10):609-617; Cook, J. A., Shah, K. B., Quader, M. A., Cooke, R. H., Kasirajan, V., and 4 others 2015. “The Total Artificial Heart,” Online, Journal of Thoracic Disease (Hong Kong, China) 7(12):21 72-80; Spiliopoulos, S., Dimitriou, A. M., Serrano, M. R., Guersoy, D., Autschbach, R., and 3 others 2015. “Percutaneous Dilatational Tracheostomy following Total Artificial Heart Implantation,” Online, Interactive Cardiovascular and Thoracic Surgery (Oxford, England) 21(1):117-118; Gerosa, G., Scuri, S., Iop, L., and Torregrossa, G. 2014. “Present and Future Perspectives on Total Artificial Hearts,” Online, Annals of Cardiothoracic Surgery (Hong Kong, China) 3(6):595-602; Lund, L. H., Gabrielsen, A., Tiren, L., Hallberg A., El Karlsson, K, and Eriksson, M. J. 2012. “Derived and Displayed Power Consumption, Flow, and Pulsatility over a Range of HeartMate II Left Ventricular Assist Device Settings,” American Society for Artificial Internal Organs Journal 58(3):183-190).

The implantation and use of mechanical circulatory support devices and a conventional heart transplant for that matter, also carry serious risks which the compound vascular bypass method eliminates (see, for example, Chau, V. Q., Oliveros, E., Mahmood, K., Surach, C., Roldan, J., and 5 others 2021. “Troubleshooting Total Artificial Heart: Novel Use of Implantable Hemodynamic Monitor,” Online, Journal of the American College of Cardiology. Case reports (Amsterdam, North Holland, Netherlands) 3(7):1024-1028; Potapov, E. V., Antonides, C., Crespo-Leiro, M. G., Combes. A., Farber, G., and 15 others 2019. “2019 EACTS [European Association for Cardio-thoracic Surgery] Expert Consensus on long-term Mechanical Circulatory Support,” European Journal of Cardio thoracic Surgery 56(2): 230-270; Kremer, J., Farag, M., Arif. R., Brcic, A., Sabashnikov, A., and 6 others 2016. “Total Artificial Heart Implantation after Undifferentiated High-grade Sarcoma Excision,” Medical Science Monitor Basic Research 22:128-131; Ramirez, A., Riley, J. B., and Joyce, L. D. 2016. “Multi-targeted Antithrombotic Therapy for Total Artificial Heart Device Patients,” The Journal of Extra-corporeal Technology 48(1):27-34; Cook, J. A., Shah, K. B., Quader, M. A., Cooke, R. H., Kasirajan, V., and 4 others 2015. “The Total Artificial Heart,” Journal of Thoracic Disease 7(12):2172-2180-erratum, 2017, Journal of Thoracic Disease 2017 9(3):E342; Kilic, A., Acker, M. A., and Atluri, P. 2015. “Dealing with Surgical Left Ventricular Assist Device Complications,” Journal of Thoracic Disease 7(12):2158-2164; additional references shortly below).

In contrast to the power requirements of assist devices and total artificial hearts, toggling vascular servovalves, sometimes even unnecessary or infrequent, requires a small fraction of the power. The power source is smaller, can consist of batteries such as rechargeable lithium-ion button cells housed in a body surface port, the interval for recharging much longer, and there is no incision into either the donor or recipient heart.

Ventricular assist devices have been reduced in size for use in children under the age of 10. Although progress toward this end is being made, this is not so for total artificial hearts (see, for example, Fukamachi, K., Karimov, J. H., Byram, N. A., Sunagawa, G., Dessoffy, R., Miyamoto, T., and Horvath, D. J. 2018. “Anatomical Study of the Cleveland Clinic Continuous-flow Total Artificial Heart in Adult and Pediatric Configurations,” Journal of Artificial Organs 21(3):383-386; Kirklin, J. K. 2015. “Advances in Mechanical Assist Devices and Artificial Hearts for Children,” Current Opinion in Pediatrics 27(5):597-603; Park, S. S., Sanders, D., Smith, B. P. Ryan, J., Plasencia, J., and 10 others 2014. “Total Artificial Heart in the Pediatric Patient with Biventricular Heart Failure,” Perfusion 29(1):82-88; Slepian, M. J., Alemu, Y., Girdhar, G., Soares, J. S., Smith, R. G., Einav. S., and Bluestein, D. 2013. Op cit. above in this section).

Moreover, due to the larger number and complexity of parts and the surgical preparation of the native heart to place a mechanical assist device, these are susceptible to more diverse and numerous complications than are the relatively simple vascular servovalves used in a double heart transplant (see, for example, Vaidya, Y., Riaz, S. and Dhamoon, A. S. 2020, Op cit., section 2a; Han, J. and Trumble. D. R. 2019. “Cardiac Assist Devices: Early Concepts, Current Technologies, and Future Innovations,” Bioengineering (Basel, Switzerland) 6(1). pii: E18; Reiss, N., Schmidt, T., Boeckelmann, M., Schulte-Eistrup, S., Hoffmann, J. D., Feldmann, C., and Schmitto. J. D. 2018. “Telemonitoring of Left-ventricular Assist Device Patients-Current Status and Future Challenges,” Journal of Thoracic Disease 10 (Supplement 15):S1794-S1801; Prinzing, A., Herold, U., Berkefeld, A., Krane, M., Lange, R., and Voss, B. 2016. “Left Ventricular Assist Devices-Current State and Perspectives,” Journal of Thoracic Disease 8(8):E660-E666; Rodriguez, L. E., Suarez, E. E., Loebe. M., and Bruckner, B. A. 2013. “Ventricular Assist Devices (VAD) Therapy: New Technology, New Hope?,” Methodist Debakey Cardiovascular Journal 9(1):32-37; Lund, L. H., Gabrielsen, A., Tiren, L., Hallberg. A., El Karlsson, K., and Eriksson, M. J. 2012. “Derived and Displayed Power Consumption, Flow, and Pulsatility over a Range of HeartMate II Left Ventricular Assist Device Settings,” American Society for Artificial Internal Organs Journal 58(3):183-190).

The ultimate bane for mechanical circulatory devices-which does not pertain to a transplant—is the need to supply the device with sufficient power; a total artificial heart with fully implanted rechargeable power source not requiring that the patient remain within range of a transcutaneous energy transfer source providing a recharging interval of a week is not in the offing. While various schemes to include transcutaneous energy transfer show promise (Waters, B. H., Park, J., Bouwmeester, J. C., Valdovinos, J., Geirsson, A., and 3 others 2018. “Electrical Power to Run Ventricular Assist Devices Using the Free-range Resonant Electrical Energy Delivery (FREE-D) System,” Online, Journal of Heart and Lung Transplantation (New York, New York) 37(12):1467-1474), attempts to provide nuclear powered artificial hearts appearing in the literature more than forty years ago and more recently ventricular assist devices are no longer mentioned:

“ . . . the critical problems of heat dissipation and safety concerns regarding nuclear element leakage eventually led to termination of the project” (Han, J. and Trumble, D. R. 2019. “Cardiac Assist Devices: Early Concepts, Current Technologies, and Future Innovations,” Bioengineering (Basel, Switzerland) 6(1). pii: E18)(see also; for example, Tchantchaleishvili. V., Bush, B. S., Swartz, M. F., Day, S. W., and Massey, H. T. 2012. “Plutonium-238: An Ideal Power Source for Intracorporeal Ventricular Assist Devices?,” American Society for Artificial Internal Organs Journal 58(6):550-553; Magovem, G. J., Park, S. B., Magovern, G. J. Jr., Christlieb, I. Y., and Kao, R. L. 1987. “Mechanical Circulatory Assist Devices,” Texas Heart Institute Journal 14(3):276-283; Kiselev. I. M., Dubrovskil, G. P., Mosidze, T. G., and Bazhanov, A.Iu. 1983. “Prospects for Using Implanted Systems of Assisted Circulation and Artificial Heart with a Radioisotope Power Source (Biomedical, Thermal, and Radiation Aspects),” Artificial Organs (Cambridge, Massachusetts) 7(1):117-121; Shumakov, V. I., Griaznov, G. M., Zhemchuzhnikov, G. N., Kiselev. I. M., and Osipov, A. P. 1983. “Implanted Artificial Heart with Radioisotope Power Source,” Artificial Organs 7(1):101-106; Kiselev, I. M., Kremnev, V. A., Sadov, V. V., and Spiridonov, V. A. 1976. “Power Units of Implanted Artificial Heart and Assisted Circulation System,” in Russian with English abstract at Pubmed, Meditsinskaia Tekhnika [Biomedical Engineering] (4):50-56; Huffman, F. N. and Norman, J. C. 1974. “Nuclear-Fueled Circulatory Support Systems IV: Radiologic Perspectives,” Cardiovascular Diseases (Houston, Texas) 1(5):463-476; Huffman, F. N., Hagen, K. G., Whalen, R. L., Fuqua, J. M., and Norman, J. C. 1974. “Intracorporeal Heat Dissipation from A Radioisotope-Powered Artificial Heart,” Cardiovascular Diseases (Houston, Texas) 1(4):343-368; Norman J. C., Molokhia, F. A., Harmison, L. T., Whalen, R. L., and Huffman, F. N. 1972. “An Implantable Nuclear-fueled Circulatory Support System. I. Systems Analysis of Conception, Design, Fabrication and Initial in Vivo Testing,” Annals of Surgery (Philadelphia, Pennsylvania) 176(4):492-502: Mott. W. E. and Cole, D. W. Jr. 1972. “Development of a Nuclear-powered Artificial Heart,” Transactions of the American Society for Artificial Internal Organs 18(0):152-158; Sandberg, G. W. Jr., Lee. R. C., Pegg. C. A., Huffman, F. N., and Norman, J. C. 1969. “Simulated Radiation Fields from Plutonium-238-fueled Artificial Hearts: Long-term Effects of Intracorporeal Strontium 90-americium 241-beryllium Sources,” Surgical Forum 20:194-196).

Because a pump device propels rather than gates the blood as does toggling with the occasional release of a drop of a heparin, for example, to prevent, and/or a thrombolytic such as streptokinase, urokinase, or tissue plasminogen activator to break up, clot and/or a nitrate to prevent and relax a vasospasm, flow resistant events such as obstructions, due to thrombus or vasospasm, for example, can unexpectedly change the power requirement—“When flow is obstructed unrelated to contact with the rotor, power is reduced, whereas power is increased (and flows decreased) when thrombus forms on the rotor” (Sen, A., Larson, J. S., Kashani, K. B., Libricz, S. L., Patel, B. M., and 4 others 2016. “Mechanical Circulatory Assist Devices: A Primer for Critical Care and Emergency Physicians,” Online, Critical Care (London, England) 20(1):153)(see also, for example, (Feih, J., Weierstahl, K., Juul, J., Brazauskas. R., Held-Godgluck, B., and Rinka, J. 2024. “Retrospective Evaluation of Inpatient Warfarin Management Practices in Patients Immediately following Left Ventricular Assist Device Implantation,” Journal of Pharmacy Technology (Thousand Oaks, California) 40(5):215-222; Kuczaj, A., Skrzypek, M., Hudzik, B., Kaczmarski, J., Pawlak, S., Hrapkowicz, T., and Przybyłowski, P. 2024. “Fibrin Clot Permeability (Ks) in Patients on Left Ventricular Assist Device,” Online, [Nature] Scientific Reports (London, England) 14(1):20193; Lombardi, M., Bonora, M., Baldetti, L., Pieri. M., Scandroglio, A. M., and 4 others 2023. “Left Ventricular Assist Devices Promote Changes in the Expression Levels of Platelet MicroRNAs,” Online, Frontiers in Cardiovascular Medicine (Lausanne, Switzerland) 10:1178556; Saeed, D., Feldman, D., Banayosy, A. E., Birks. E., Blume, E., and 81 others 2023. “The 2023 International Society for Heart and Lung Transplantation Guidelines for Mechanical Circulatory Support: A 10-year Update,” Online, Journal off Heart and Lung Transplantation (New York, New York) 42(7):e1-e222; Zaloznik Djordjevic, J., Özkan, T., Goncz, E., Ksela, J., Mockel, M., and Stmad, M. 2023. “Common Complications and Cardiopulmonary Resuscitation in Patients with Left Ventricular Assist Devices: A Narrative Review,” Online, Medicina (Kaunas, Lithuania/Basel, Switzerland) 59(11):1981; Agnieszka, K., Bartosz, H., Jacek, K., and Piotr, P. 2022. “Hemostasis Disturbances in Continuous-flow Left Ventricular Assist Device (CF-LVAD) Patients-Rationale and Study Design,” Online, 11(13): 3712; MDPI [Multidisciplinary Digital Publishing Institute] Journal of Clinical Medicine (Basel, Switzerland) 11(13): 3712; Jefferson, H. L., Kent, W. D. T., MacQueen, K. T., Miller, R. J. H., Holloway, D. D., and Fatehi Hassanabad, A. 2021. “Left Ventricular Assist Devices: A Comprehensive Review of Major Clinical Trials, Devices, and Future Directions,” Journal of Cardiac Surgery (Hoboken, New Jersey) 36(4):1480-1491; Schmack, B., Grossekettler, L., Zeriouh, M., Weymann, A, Mohite. P., and 5 others 2019. “It Keeps on Turning: Effects of Prolonged Long-term Left Ventricular Assist Device Support as a Bridge to Heart Transplantation,” International Journal of Artificial Organs (Thousand Oaks, California) 42(2):65-71; Bartoli, C. R., Zhang, D. M., Hennessy-Strahs, S., Kang. J., Restle, D. J., and 3 others 2018. “Clinical and in Vitro Evidence that Left Ventricular Assist Device-induced von Willebrand Factor Degradation Alters Angiogenesis,” Online, Circulation. Heart Failure (Hagerstown, Maryland) 11(9):e004638; Muslem, R., Caliskan, K., and Leebeek, F. W. G. 2018. “Acquired Coagulopathy in Patients with Left Ventricular Assist Devices,” Online, Journal of Thrombosis and Haemostasis (New York, New York) 16(3):429-440; Trinquero. P., Pirotte, A., Gallagher, L. P., Iwaki, K. M., Beach, C., and Wilcox, J. E. 2018, Op cit., section 2a; Shah, P., Tantry, U. S., Bliden, K. P., and Gurbel, P. A. 2017. “Bleeding and Thrombosis Associated with Ventricular Assist Device Therapy,” Journal of Heart and Lung Transplantation (New York, New York) 36(11):1164-1173; Salerno, C. T., Sundareswaran, K. S., Schleeter, T. P., Moanie, S. L., Farrar, D. J., and Walsh, M. N. 2014. “Early Elevations in Pump Power with the HeartMate II Left Ventricular Assist Device Do Not Predict Late Adverse Events,” Journal of Heart and Lung Transplantation (New York, New York) 33(8):809-815; Stulak, J. M., Lee, D., Haft, J. W., Romano, M. A., Cowger, J. A., and 3 others 2014. “Gastrointestinal Bleeding and Subsequent Risk of Thromboembolic Events During Support with a Left Ventricular Assist Device,” Online, Journal of Heart and Lung Transplantation (New York, New York) 33(1):60-64; Rothenburger, M., Wilhelm, M. J., Hammel, D., Schmidt, C., Tjan, T. D., and 3 others 2002. “Treatment of Thrombus Formation Associated with the MicroMed DeBakey VAD Using Recombinant Tissue Plasminogen Activator,” Circulation 106(12 Supplement 1):1189-1192).

Biventricular assist devices and artificial hearts eliminate right heart failure but do so at the expense of compelling the patient to wear obtrusive batteries and a system control unit, and undergo exposure to considerable risk during implantation and throughout the life of the device (see, for example, Lund, L. H., Gabrielsen, A., Tiren, L., Hallberg. A., El Karlsson, K., and Eriksson, M. J. 2012. “Derived and Displayed Power Consumption, Flow, and Pulsatility over a Range of HeartMate II Left Ventricular Assist Device Settings,” American Society for Artificial Internal Organs Journal 58(3):183-190).

The practical result of the increase in power consumption needed to toggle valves is that a rechargeable battery as power source can be relegated to a small port at the body surface or paracorporeally, or positioned subcutaneously for transcutaneous, or transdermal recharge. The AbioCor experimental total artificial heart with transcutaneously recharged battery has been studied but not entered into clinical use. Unless relegated to a paracorporeal battery pack, the size, weight, and recharging interval for an implanted battery or batteries to power such a device would likely cause discomfort as well as inconvenience even before medical complications became apparent.

Total artificial hearts have the potential to avert the relatively high risk of stroke, arrhythmias, and valve dysfunction experienced with left and biventricular assist devices (see, for example, Melton, N., Soleimani, B., and Dowling, R. 2019. “Current Role of the Total Artificial Heart in the Management of Advanced Heart Failure,” Current Cardiology Reports 21(11):142; Beaupré, R. A., Frazier, O. H., and Morgan, J. A. 2018. “Total Artificial Heart Implantation as a Bridge to Transplantation: A Viable Model for the Future?,” Expert Review of Medical Devices 15(10):701-706; Cook, J. A., Shah, K. B., Quader, M. A., Cooke, R. H., Kasirajan, V., and 4 others 2015. “The Total Artificial Heart,” Journal of Thoracic Diseases 7(12):2172-2180).

The considerably greater shear stress of turbine continuous flow devices in a patient with von Willebrand disease as in Heyde's syndrome which causes bleeding from the gut due to the high shear stress of aortic valvular stenosis which cleaves the von Willebrand platelet aggregation factor would not apply to toggling. Any such tendency would be counteracted through the directly targeted release of an anticoagulant drip via a drugline inserted in a vascular valve accessory channel to dispose of clotting as a problem. These factors and the need for systemie anticoagulation make such devices useful as bridges to transplantation but nonessential when a method for reducing the shortage of hearts acceptable for transplantation is available.

Such includes the use of mechanical assist devices (see, for example, Han, J. and Trumble, D. R. 2019. “Cardiac Assist Devices: Early Concepts, Current Technologies, and Future Innovations,” Bioengineering (Basel) 6(1). pii: E18; Mancini, D. and Colombo, P. C. 2015. “Left Ventricular Assist Devices: A Rapidly Evolving Alternative to Transplant,” Journal of the American College of Cardiology 65(23):2542-2555). Until the sufficiency of chute toggling is confirmed, the temporary use of wearable or implantable cardioverter-defibrillators for each of two hearts positioned in a single circulatory system is a precaution against a cardiac hypovolemia-induced sudden arrest. The assist heart is chosen to complement the primary heart so as to obtain an acceptable ventricular absolute stroke volume, and the setpoint about which the diversion chutes are toggled is adjusted to further normalize this value.

The addition of a second heart differs from a heterotopic, or ‘piggyback’ transplant, in that a closed-heart procedure, neither heart is entered much less traumatized. Almost none of the conventional causes for adverse sequelae—the shock of death, recipient and donor heart bisection to retain the native atria, general anesthesia, cardioplegia, cross-clamping, cardiopulmonary support, open-heart surgery, the use of general rather than regional anesthesia, and in particular, ischemia, known to result in the risk of dysrhythmia and reperfusion injury proportional to the time thereof is used.

Another difference is that in most instances, it will be difficult or impossible to position two intact hearts in the inferior mediastinal space as permanent except on an interim basis. Positioned abdominally or infrarenally, the added heart must have had the blood conveying lines, or mainlines, sized to allow such a nonadjacent positioning. Connection is not locally to the abdominal aorta and inferior vena cava as in some animal models used to study immunization and remodeling and atrophy of the myocardium due to volume unloading following placement of a left ventricular assist device, but follows the normal pattern of flow through the assist heart.

These studies delineate sequelae that might be used to distinguish the results of conventional and metered switch double heart heterotopic heart transplantation (see, for example, Schaefer, A., Schneeberger, Y., Schulz, S., Krasemann, S., Werner, T., and 12 others 2019. “Analysis of Fibrosis in Control or Pressure Overloaded Rat Hearts after Mechanical Unloading by Heterotopic Heart Transplantation,” (Nature Research) Scientific Reports 9(1):5710; Westhofen, S., Jelinek, M., Dreher. L., Biermann, D., Martin, J., and 4 others 2019. “The Heterotopic Heart Transplantation in Mice as a Small Animal Model to Study Mechanical Unloading-Establishment of the Procedure, Perioperative Management and Postoperative Scoring,” Online, PLAS [Public Library of Science] One 14(4):e0214513; Pokomý, M., Mrázová, I., Kubátová, H., Pitha, J., Malý, J., and 9 others 2019, Op cit. above in this section; Pokomý, M., Mrázová, I., Šochman, J., Melenovský, V., Malý, J., and 9 others 2018, Op cit. above in this section; Benke, K., Sayour, A. A., Matyas, C., Agg. B., Nemeth, B. T., and 7 others 2017. Op cit. above in section 2b(1)(b)(b5); entitled Heterotopic Double Heart Metered Compound Vascular Bypass, or Switched-organ Transplantation [and so on]. Kleinert. E., Langenmayer, M. C., Reichart. B., Kindermann, J., Griemert. B., and 11 others 2016. “Ribonuclease (RNase) Prolongs Survival of Grafts in Experimental Heart Transplantation,” Journal of the American Heart Association (Oxford, England) 5(5). pii: e003429; Fu. X., Segiser, A., Carrel. T. P., Tevaearai Stahel. H. T., and Most. H. 2016. “Rat Heterotopic Heart Transplantation Model to Investigate Unloading-induced Myocardial Remodeling,” Online, Frontiers in Cardiovascular Medicine 3:34. eCollection 2016; Schaefer, A., Schneeberger, Y., Stenzig, J., Biermann, D., Jelinek, M., and 4 others 2016. “A New Animal Model for Investigation of Mechanical Unloading in Hypertrophic and Failing Hearts: Combination of Transverse Aortic Constriction and Heterotopic Heart Transplantation,” Online, PLoS [Public Library of Science] One 11(2):e0148259; Liu, Y., Maureira, P., Gauchotte, G., Falanga, A., Marie, V., and 5 others 2015. “Effect of Chronic Left Ventricular Unloading on Myocardial Remodeling: Multimodal Assessment of Two Heterotopic Heart Transplantation Techniques,” Journal of Heart and Lung Transplantation (New York, New York) 34(4):594-603; Carr, C. A., Ball, D., Tyler, D. J., Bushell. A., Sykes. A., Clarke, K., and Evans, R. D. 2014. “VaryingDegrees of Ventricular Unloading in the Heterotopic Rat Heart Transplant Model Demonstrated by Magnetic Resonance Imaging,” International Journal of Biomedical Science 10(4):223-228; Ibrahim, M., Navaratnarajah, M., Kukadia, P., Rao, C., Siedlecka, U., and 5 others 2013. “Heterotopic Abdominal Heart Transplantation in Rats for Functional Studies of Ventricular Unloading,” Journal of Surgical Research 179(1):e31-e39; Didie, M., Biermann, D., Buchert, R., Hess, A., Wittkopper, K., and 7 others 2013. “Preservation of Left Ventricular Function and Morphology in Volume-loaded versus Volume-unloaded Heterotopic Heart Transplants,” American Journal of Physiology. Heart and Circulatory Physiology 305(4):H533-H541: Wen, P., Wang, X., Li, T., Zhang, B., Sun, X., and 6 others 2013. “Establishment of a Novel Volume-loaded Heterotopic Heart Transplantation Model in Rats,” Journal of Surgical Research 183(1):435-441; Oriyanhan, W., Tsuneyoshi, H., Nishina, T., Matsuoka, S., Ikeda. T., and Komeda, M. 2007. “Determination of Optimal Duration of Mechanical Unloading for Failing Hearts to Achieve Bridge to Recovery in a Rat Heterotopic Heart Transplantation Model,” Journal of Heart and Lung Transplantation (New York, New York) 26(1):16-23; Tsuneyoshi, H., Oriyanhan, W., Kanemitsu, H., Shiina, R., Nishina, T., and 3 others 2005. “Heterotopic Transplantation of the Failing Rat Heart as a Model of Left Ventricular Mechanical Unloading toward Recovery,” American Society for Artificial Internal Organs Journal 51(1):116-120). Positioning thus least preferred and resorted to only when any more favorable location is contraindicated, in the lower abdomen, or infrarenally, where no protective bony cage-surround is present, an externally worn medieval armor plackart (planckart, placcard, placcate) should be used before engaging in any activity that might result in injury. If such activity is frequent due to employment as a driver, for example, then as described below, a stainless steel heart cage implanted with the donor heart is used.

Should the native or previously transplanted heart, that is, the primary heart, already having undergone acute decompensated failure then further deteriorate to American College of Cardiology/American Heart Association Stage D or New York Heart Association Class IV, and no sound replacement heart is available, it is replaced with a less than ideal heart by using the vascular servovalves to divert all blood flow to the heterotopic added or assist heart which then drives the blood so that the native heart can be removed. If the assist heart cannot maintain normal blood pressure, then left and/or right ventricular assist devices are placed as necessary. Toggling applied only to the aortic and pulmonary arteries of the primary heart, it is now, with all blood having been diverted to the heterotopic assist heart, stopped, and the valve-jackets disconnected from the orthotopic primary heart which is removed.

Diversion to the primary heart having been stopped, blood does not spill out of the mainlines when disconnected. The new heart is then connected to the mainlines and the diversion chutes retracted to the toggling setpoint before toggling is reinitiated. Such replacement is best sequential, the heterotopic procedure described above performed before the native heart is removed and a second transplant placed, allowing recovery and the attainment of stability and the patient allowed to fully recover before proceeding with the second transplant. The first transplant is by the sudden switch technique, the second, if the condition of the patient permits, is by the metered switch technique. If it eventuates that the assist heart requires replacement, the procedure is the same.

When it appears that the patient is declining so that death is imminent, the two less than ideal hearts are transplanted to replace the native heart in a single sudden switch operation. To transplant a pair of hearts requires that the two graft hearts have been previously paired by meter switch transplanting the heart of one deceased donor on somatic support into a second donor as indicated above. In this circumstance, where the paired hearts will reside in the second donor on a short term basis, positioning can be in the chest with the sternum left open. That positioning in the living recipient will likely require a heterotopic positioning must be kept in mind when gauging the length of the mainlines.

Even with toggling, the volume of blood in a recipient, resynchronizing implants notwithstanding, will not be sufficient to supply three hearts, so that all three could suddenly arrest. For this reason, the sudden switch technique is used to abruptly transfer the full volume of blood from the native or earlier transplanted heart of the living recipient to the hearts previously paired in one of the deceased donors; blood is never shared between the recipient and donor hearts. The full complement of recipient blood suddenly switched to the pair then requires to be shared. Before or after transplantation, paired hearts must have the blood supply toggled to prevent cardiac hypovolemia. This had been accomplished by initiating to venae cavae toggling of the arterial valves in the earlier deceased recipient native or primary heart when the second heart was added. Now when the paired hearts are to be transplanted into the living recipient, toggling in the second deceased donor must be switched to the recipient.

To accomplish the replacement of the recipient heart with the duo having earier been meter switched with immune tolerance induction in the earlier deceased recipient, now the deceased donor, the switch is made with the duo treated as a unit, as if it were a single heart. Accordingly, servovalve diversion jackets are placed just outside the in- and outflow jackets of the duo and the heart of the recipient and the circulation of the recipient suddenly switched to the duo. Even though such a double heart transplant begins with a sudden switch from the native to the paired hearts, this action is controlled by a microcontroller Bluetooth signal transmitted to the microcontrollers controlling the servovalves in both the donor double heart and the diseased native heart of the recipient. If necessary, power is replenished by transcutaneous energy transfer.

Accomplishment of the initial switch of recipient blood flow from the native to the donor hearts sudden notwithstanding, servo-rather than solenoid-driven valves are needed to simultaneously stop the toggling action of the duo needed to sufficiently perfuse more than one heart at a time while in the donor and resume toggling of the diversion chutes in the arterial valves when the duo is switched to the recipient. Accordingly, such a simultaneous two-for-one transplant is accomplished by placing servovalve diversion jackets on the corresponding inflow and outflow vessels of the native and paired donor hearts. At the instant blood flow is switched from the recipient to the donor pair, the snap-action plunger switch used by the operator generates Bluetooth signals received by the donor microcontroller to stop, and by the recipient microcontroller to start toggling.

Whereas in metered compound vascular bypass, or sudden switched-organ transplantation where an external controller coordinates the transfer across both recipient and donor, here where the transfer is sudden, this is not the case. The switch is therefore between controllers as well as circulatory systems. That is, at the moment of switching, control over the process is handed off from the donor to the recipient microcontroller. Thus, while the sinking condition of the patient necessitates transplantation of the pair into the recipient using the sudden switch technique, the graft pair had been joined using the metered switch technique, at least partially reconciling the one to the other from an immunological standpoint.

### Heart or Other Solid Organ Transplants

Schematically depicted in FIG. 15, in a metered compound vascular bypass heart or other solid organ transplant, the diversion chutes 18 of the servovalves are initially completely retracted with the graft organs and circulatory systems of the donor and recipient isolated from one another. The control system then commences to gradually extend all of the diversion chutes to initiate reciprocal cross-circulation. The gradual increase in donor-recipient reciprocal blood flow is monitored for signs of rejection, remediating and immune tolerance inducing, and if necessary, anticoagulant such as a heparin, medication released into the affected servovalves through the drugline each incorporates, hence their respective bloodlines, or mainlines.

The control system prescription-program includes instructions to adjust the rate or if necessary, halt the gradual merging of the donor and recipient circulatory systems. Rejection analyte sensors are addressed above in section 2a, entitled Background of the Invention. General, under subsection e, entitled Graft Organ Rejection Analytes and Immunosensors. Synchronization electronics, typically a conventional pacemaker/cardioverter-defibrillator, is always in use to counteract an arrhythmia much less a sudden cardiac arrest.

If suspension is responsive to a rejection reaction, the system will execute the prescribed regimen for counteracting the obstruction until it has been overcome, but will not end the process by selecting the preferable or stronger of the organs, here by definition, the heart of the donor, for implantation. Given that the donor and recipient have been matched before the process was allowed to commence, the odds that a significant interruption would arise are few. Accordingly, for the process to not proceed to completion due to an interruption is highly improbable.

1. When a competent replacement heart is available, a failing adult or severely mal formed pediatric heart is replaced orthotopically in the chest. Except in circumstances sufficiently exigent to justify use of the sudden switch technique, transplantation is accomplished using the metered switch technique described in section 2b(1)(b), entitled Metered Compound Vascular Bypass, of Metered Switched-organ Transplantation. This allows immune tolerance induction both intrinsic as microchimerization as well as by medicinal supplementation, programmed and automatic, or manually, during transfer of the graft from the donor to the recipient. The native heart is removed by transection of its inflow and outflow vessels inside, or to the heart side of the valve-jackets, that is, proximal to the superior and inferior margins of the heart, while the graft heart is removed by transection of its inflow and outflow vessels outside, that is, superior and inferior to its valve-jackets.

Thus, when the graft heart is positioned in the recipient, each inflow and outflow vessel passes blood through two consecutive valve-jackets, that remaining on each such native vessel, to which is added that on each such corresponding inflow and outflow vascular stump of the graft heart. While the blood-conducting mainlines and both valve-jackets can be left as prostheses without anastomosing the graft stumps to the native vessel stumps when these are diseased, it is more usual to:
1. Anastomose the graft stumps to the native stumps.
2. Fully retract the diversion chutes so that blood will flow through the anastomoses, eliminating the need for the mainlines, which are
3. Removed along with
4. One of the two valve-jackets on the inflow and one on the outflow vessels, the other inflow and outflow valve jacket left in place. The remaining inflow (here vena caval) valve jacket is left in place so that its accessory channel or channels can be used to directly pipeline target medication to the graft organ and if anastomosed, the suture lines, and the remaining outflow (the aorta and pulmonary artery) jacket to deliver a reversal agent if necessary to prevent the drug from provoking adverse side effects or tissue reactions in nontargeted tissue.

While convergent or Y-configured anastomoses would allow the vascular stumps of both the native and a second heart to be anastomosed, from a practical standpoint, this tedious process, requiring the addition of homograft or tissue engineered vessels even when the second heart could be positioned in the chest would extend procedural time greatly. For this reason, only replacement of the native heart, that is, a single orthotopic (with or without a second, heterotopic) heart transplant, reasonably allows the native heart inflow and outflow stumps to be simply end-to-end anastomosed to the graft stumps.

Adding an assist heart to that transplanted first requires returning the diversion valves to the half-way position to divert blood to the additional heart, whether positioned orthotopically or heterotopically, connection between the two hearts then continued indefinitely through the valve-jackets and mainlines. The reason blood inflow and outflow are not limited to half the normal volume with predictable adverse consequences is that toggling of the chutes sends the full complement of blood to each heart frequently enough to approximate normal flow and the two hearts should adapt to a slight deficit thereof, as addressed below.

However, as human growth hormone, or somatotropin, circulates in the blood, anastomosis is not essential for a pediatric transplant to grow. That switch transplantation completely avoids incisional trauma or ischemia to the transplant organ or organ, here, a heart of hearts, is a factor in allowing the use of less than ideal hearts to be placed. Another factor granting latitude is that within limits, both over- and undersized hearts have been established to adapt to a change in load as not to produce pulmonary vascular hypertension beyond an initial interval during which the hypertension can be controlled medically.

2. When the left and/or right ventricular absolute stroke volumes of the native heart are reduced and a sound replacement heart is unavailable, then rather than to replace the native heart, a second marginal heart is added to the native marginal heart where the added heart is positioned in the chest if possible; if not, then heterotopically, in the abdomen or infrarenally. In either event, the native heart is not entered and only has valve-jackets placed about its major vessels, avoiding any further degradation in its competency due to surgical trauma. If not positioned in the chest, the added heart is positioned heterotopically, but in either case, the added heart remains vessel-to-vessel connected to the native heart so that there is no change in its inflow and outflow pattern.

The result might be characterized as equivalent to a more conventional ‘piggyback’-type heterotopic transplant, with ventricular function doubled but without ischemia, cardioplegia, cardiopulmonary bypass, cross-clamping, general anesthesia, entry into, or wounding of either heart. Should handling the hearts following removal with inflow and outflow vessels from their native milieu provoke vasospasm, the immediate access to these through the accessory channels makes the targeted delivery of fast-acting nitrates and/or botulinum toxin to the site or sites a relatively simple matter.

When time allows, a presumptive predisposition toward a disruptive midprocedural vasospasm may justify the preprocedural administration of a calcium channel blocker antihypertensive such as nifedipine, nicardipine, isradipine, diltiazem, or verapamil, and/or a renin-angiotensin system inhibitor (See, for example, Park, T., Park, J. Y., Rha, S. W., Seo, H. S., Choi, B. G., and 15 others 2017. “Impact of Diltiazem Alone versus Diltiazem with Nitrate on Five-year Clinical Outcomes in Patients with Significant Coronary Artery Spasm,” Yonsei Medical Journal 58(1):90-98; Choi, B. G., Jeon, S. Y., Rha, S. W., Park, S. H., Shim, M. S., and 16 others 2016. “Impact of Renin-Angiotensin System Inhibitors on Long-term Clinical Outcomes of Patients with Coronary Artery Spasm,” Journal of the American Heart Association (Oxford, England) 5(7). pii: e003217).

Especially for placement in the chest, a second smaller heart to attain an adequate combined ejection fraction is more readily accommodated. Using the compound vascular bypass method, a less than ideal heart can be used to assist an end-stage failing native heart, which is advantageous in parts of the world where a ventricular assist device or artificial heart is unaffordable. Placed separately and intact as to be mostly but not entirely separate for diagnostics and therapeutics, the second heart will remain donor-to-recipient vessel-to-vessel connected to that native, so that the flow pattern through both hearts will be normal.

Accordingly, with the continuous medicinal and if necessary electronic support provided by the fully implanted automatic response system, the possibility for rehabilitation of the native heart should emerge as improved even in a conventional context. However, whether conventional or conventional heterotopic, the unitary heart is not recoverable for retransplantation into another patient. The procedure for recovery of the added heart following a compound vascular bypass heart transplant is described above in section 2b(1)(b)(b5), entitled Heterotopic Double Heart Metered Compound Vascular Bypass, or Switched-organ Transplantation [and so on], in the subsection entitled Seamless and Ischemia-free Explantation and Transfer of an Allograft Second Heart from a First to a Second recipient, and is implemented using the circuit shown in FIG. 15B.

When a graft organ is recovered, leaving the side-entry vascular valves or servovalves in place preserves their druglines for continued use by the implanted disorder response system to target mediation to the site of the repair. The results of conventional heterotopic heart transplantation indicate that two hearts adapt not just individually but as a pair. In a single organ replacement using the sudden switch technique, the native organ is suddenly cut off from, and the replacement organ inserted into, the circulatory system of the recipient. Except in exigent circumstances which justify the sudden switch technique, transplantation is always by the metered switch technique, which to allow immune tolerance induction concurrent with switching between recipient and donor organs, is made to progress gradually rather than abruptly.

As will be addressed shortly, in order to pass blood to and from two hearts, any double heart configuration requires that the valves be movable to the midpoint, whence these can be fully advanced and fully retracted. Bistable, solenoid-driven valves move the diversion chute between fully extended or closed to divert all flow-through to fully retracted or fully open, to allow all flow to pass without diversion-perfect for sudden compound vascular bypass, or sudden switched-organ transplantation, but not capable of supporting a double heart configuration, which to provide blood to both hearts, requires that the diversion valves be continuously variable in the degree of extension into the lumen.

This is so whether a second heart is added to that native or the native heart is replaced with two hearts. For this reason, when applied to two hearts, sudden compound vascular bypass, or sudden switched-organ transplantation also requires the use of vascular servovalves. When a second heart is added to that native, the lines are connected as in a one-for-one replacement of the native heart, except that rather than to replace it, the native heart is left in place and only the added heart removed for placement in the recipient.

Then, if the diversion chutes were left at the midway positions to pass half the normal complement of blood to either of the two hearts, the cardiac hypovolemic condition would likely induce dysrhythmias in both. To substantially if not entirely overcome the delivery to either heart of only half the normal complement of blood, which if not immediately reversed would risk cardiac hypovolemia and arrhythmia and even sudden arrest, the diversion chutes are frequently toggled first, between the extent to which they have advanced to fully retracted, or open, then fully closed in synchrony with a regular number of systoles, pausing momentarily at the half-way point, cardioverter-defibrillators placed in both hearts.

The use of artificial cardiac pacemakers, implantable cardioverter-defibrillators, and automated external defibrillators in conjunction with conventional heterotopically transplanted hearts is not addressed in the literature. A two in one artificial cardiac pacemaker or implantable cardioverter-defibrillator with leads to either of two hearts to protect both or to maintain synchrony between hearts where one is in the chest and the other is positioned infrarenally is also not addressed. The paddles placed in contact with the skin and current passed from one to the other, use of an automated external defibrillator to selectively treat one of two hearts placed in close proximity should pose no problem but is not addressed in the literature.

Unless the over- or undersizing of the added heart is considerable, the clinically established adaptability of over- and undersized heart transplants indicates that adjustment to past or short of the halfway point as set point or reference datum for chute extension to accommodate such an added heart may be unnecessary and inadvisable. This because were the toggling action to succeed in accomplishing the volumetric load normal for the added heart despite the fact that it is off-sized, this would likely avert problems of hyper or hypotension and dysrhythmia, but prove counterproductive in removing the inducement for the added heart to adapt. Since it is desirable from the standpoint of power conservation to achieve adaptation so that the toggling action need not continue indefinitely, it is best to encourage adaptation by intermittently stopping toggling to rest at the halfway rather than the size offset point for progressively longer intervals.

Even when anastomosed, retention of the servovalves and bloodlines should be retained to allow the direct pipe-targeting of drugs to the anastomoses and create an alternate bypass path for blood flow should the native path become diseased or would best respond to therapy by remaining dry. Such anastomotic bypasses are shown in FIGS. 22B thru 22E, 32D, and 32F.

3. Following the addition of a better than marginal heart to a marginal native heart since further deteriorated as described above, replace the native heart with a competent allograft. That is, replace the native heart with two marginal hearts where the first heart is added to the native heart as per 2 in a first metered switch operation, and the native heart replaced with the second marginal heart in a second metered switch operation. The second heart is positioned in the chest if possible; if not, then heterotopically, in the abdomen or infrarenally.

4. Replace the native heart of the recipient with two marginal hearts, where the marginal heart of the first donor had earlier been metered switch transplanted into the second donor. These previously cross-microchimerized marginal hearts are then entered into a compound vascular bypass circuit with the end stage failing native heart of the new recipient and explanted together to replace the failing native heart of the recipient. Such replacement can be thoracic or heterotopic such as in the abdomen, in a single operation. Except in exigent circumstances which justify the sudden switch technique, transplantation is always by the metered switch technique.

5. Replace the end stage failing native heart of the recipient facing imminent death with two marginal hearts where both replacement hearts are taken from the donors and transplanted into the recipient orthotopically or heterotopically using the sudden switch technique in a single operation using intravascular diversion servovalves to allow follow-up cross-microchimerization.

Unconcerned with transplant survivability, positioning of a heterotopic second heart in the lower abdomen as practiced in immunological research, for example, connects the graft organ directly to the abdominal aorta and the inferior vena cava in a simple way that leaves the vasculature of the graft organ insufficiently perfused. However, circumstances might dictate that placement of a second heart in the lower abdomen is preferable. Trauma might have rendered other potential positions no longer suitable, and for flow to avoid the distance and resistance of gravity to the venous return back up to the chest may be apprehended as best avoided.

In response to these considerations, confluence conduits are addressed below in section 2b(1)(b)(b14), entitled Confluence Conduits: Connection of One or More Bloodlines to Flow through a Common Channel to the Same or a Different Number of Bloodlines with Self-leveling Apportionment of Outflow, and a type confluence conduit that maintains flow throughout the vasculature of the graft organ avoiding disuse atrophy while reducing the need for servovalves to one while allowing connection to the blood supply and drainage locally is addressed below in sections 2b(1)(b)(b16), entitled Unified Arterial Entry Confluence Conduit for the Heterotopic Connection of a Graft Heart in the Lower Abdomen, as well as in section 6, entitled Description of the Preferred Embodiments of the Invention. Positioning a second heart in the abdomen demands that it be enclosed within a protective casing, or cage such as that shown in FIG. 24, to prevent the graft organ from injury even as the result of a direct blow.

a. Confluence conduits are addressed below in section 2b(1)(b)(b14), entitled Confluence Conduits: Connection of One or More Bloodlines to Flow through a Common Channel to the Same or a Different Number of Bloodlines with Self-leveling Apportionment of Outflow.

b. Switchable confluence conduits when necessary to eliminate the slack of long bloodlines needed to extend between donor and recipient before placement in the body are addressed below in section 2b(1)(b)(b15), entitled Switchable Lines and Confluence Conduits: Long When Intercorporeal, to Short When Intracorporeal, Both Affording Connection of One or More Bloodlines to the Same or a Different Number of Bloodlines with Self-leveling Apportionment Outflow.

c. A general introduction to aortocaval connection is presented below in section 2b(1)(b)(b16), entitled Direct Aortocaval Connection: Unified Entry Arterial and Exit Venous Confluence Conduits for the Heterotopic Connection of a Graft Heart in the Lower Abdomen.

d. A structural description of unified entry aortocaval confluence conduits is provided below in section 6, entitled Description of the Preferred Embodiments of the Invention.

### Heterotopic Double Heart Transplantation in a Prenate, Neonate, or Infant

The point has been made that unless replaced, a severely malformed heart is unlikely to be surgically repaired so that it will provides normal circulation without multiple revisions over the course of a normal lifetime. In a neonate or infant with a severely malformed, such as a univentricular or Fallot heart, a double heart heterotopic compound vascular bypass, or switched-organ transplant is considered only when the patient is critical and a good replacement heart is unavailable. Such an operation seeks to compensate for the lack of one good heart by implanting two less than perfect hearts that working together will provide an adequate absolute stroke volume, or ejection fraction. The process is microsurgical, undertaken with the aid of binocular microscopes.

This is accomplished in three steps. The first is to orthotopically replace the malformed heart as delineated below in section 2b(1)(b)(b3), entitled Neonatal and Infant Compound Vascular Bypass, or Switched-organ, Transplantation of the Heart, which is facilitated through the use of arterial and venous confluence conduits as described below in sections 2b(1)(b)(b14), entitled Confluence Conduits: Connection of One or More Bloodlines to Flow through a Common Channel to the Same or a Different Number of Bloodlines with Self-leveling Apportionment of Outflow and 2b(1)(b)(b15), entitled Switchable Lines and Confluence Conduits: Long when Intercorporeal, to Short when Intracorporeal, Both Affording Connection of One or More Bloodlines to the Same of a Different Number of Bloodlines with Self-leveling Apportionment of Outflow, whereupon the circulation will have been essentially normalized if not with the force of aortic outflow intended.

If the first graft heart proves sufficient, the plan to proceed with a second heart is abandoned. If necessary, the second step is to allow the baby to stabilize, the process suspended with the aid of a mechanical circulatory assist device and use of the interval to induce immune tolerance through the administration of tolerance-inducing agents, and the prospective donor having been kept on life support past death and blood matched to the recipient, infusions of blood or blood products from the donor.

The need for reincision is avoided by leaving the entry wound patent while assiduously protected. The third step is to transplant the second heart into the recipient to work together with the first heart as described above in this section and shown in FIGS. 15, 16, and 17. In a tiny patient, for a second heart, even if smaller than the first, which the process does allow, to be fitted into the chest will likely require more trauma than the frail patient may be unable to tolerate.

Alternative positionings for the second heart and the connections these require are detailed below in sections 2b(1)(b)(b16), entitled Direct Aortocaval Connection: Unified Entry Arterial and Exit Venous Confluence Conduits for the Heterotopic Connection of a Graft Heart in the Lower Abdomen, 2b(1)(b)(b17), entitled Doubly Heterotopic Double Heart Compound Vascular Bypass, or Switched-organ Transplantation, and 2b(1)(b)(b18), entitled Hybrid Doubly Heterotopic Double Heart Transplantation.

Positioning in the abdomen through direct aortocaval connection is also described below in section 2b(1)(b)(b16)., entitled Direct Aortocaval Connection: Unified Entry Arterial and Exit Venous Confluence Conduits for the Heterotopic Connection of a Graft Heart in the Lower Abdomen. In this or any other position where the second, or auxiliary, heart does not have the protection of a bony enclosure, a heart cage as described below in the section that follows, 2b(1)(b)(b6), entitled Heart Cage for the Infrarenal Abdominal Positioning of a Second Heart, is used.

Familiar to heart transplantation researchers is intra-abdominal positioning of a pig-to-baboon cardiac xenograft (Goerlich, C. E., DiChiacchio, L., Zhang, T., Singh, A. K., Lewis, B., and 7 others 2020. “Heterotopic Porcine Cardiac Xenotransplantation in the Intra-abdominal Position in a Non-human Primate Model,” Online, [Nature] Scientific Reports (London, England) 10(1):10709). Also used has been an otherwise conventional pig-to-baboon Barnard-type heterotopic heart transplant of the kind wherein the allograft ventricles are anastomosed to the heart of the recipient (Bender, M., Panelli, A., Reichart, B., Radan, J., Mokelke, M., and 17 others 2024. “Hemodynamics in Pig-to-Baboon Heterotopic Thoracic Cardiac Xenotransplantation: Recovery from Perioperative Cardiac Xenograft Dysfunction and Impairment by Cardiac Overgrowth,” Online, Xenotransplantation (Copenhagen, Denmark) 31(1):e12841; Langin, M., Panelli, A., Reichart, B., Kind, A., Brenner, P., MayT, T., and Abicht, J. M. 2018. “Perioperative Telemetric Monitoring in Pig-to-Baboon Heterotopic Thoracic Cardiac Xenotransplantation,” Online, Annals of Transplantation (Smithtown, New York) 23:491-499).

2b(1)(b)(b6). Heart Cage for the Infrarenal Abdominal Positioning of a Second Heart

There is no good location for a second heart—the condition results from the application of extraordinary measures which nature could not anticipate to restore the patient to circulatory competence. After the chest, typically to the right-hand side of the native or earlier transplanted heart, the best location for positioning a second, donor, heart is in the lower, or infrarenal, abdomen. Even though a length of gut will likely need to be removed to provide sufficient space, this length will be far too short to induce short bowel syndrome and should prove relatively complication-free compared to creating space within the upper abdomen or the pelvis, where the organs are less amenable to resection, spatial restriction, and/or encroachment.

Implanting the second heart enclosed within a hard cage alleviates the patient of the need to wear a plackart when engaging in potentially injurious activity. A heart cage must provide adequate strength, light weight, compactness, and unobtrusiveness. The cage comprises a gridwork of hollow stainless steel or titanium tubing 3/16 inch or ½ centimeter in outer diameter, with a tubing wall thickness of 1/16 inch and openings of 1 inch of 2-½ centimeters, following the same outer shape (profile, contour) as the heart itself. The grid stands off at a distance of about ½ inch of 1-¼ centimeters from the heart per se, with additional clearance at the aortic arch to accommodate the descending aorta affording clearance for the interpositioning of a protective layer of perforated viscoelastic polyurethane foam bonded along the interior surface of the cage tubing.

As shown in FIG. 24, the cage, fabricated by electrical resistance welding stainless steel or titanium, consists of two complementary sides, each conformant to the shape (profile, contour) of the heart on its respective side, both sides providing more than adequate apertures and associated sectional grid hinges for the pulmonary and other vessels to pass through, each side hinged by means of an electrically welded stainless steel or titanium spring-hinge along one edge to allow opening and closing. Opposite the hinge is an electrically welded stainless steel or titanium case or chest latch locking hasp with catch plate latch that in cooperation with the hinge on the opposite side, locks the halves in flush position.

Accordingly, sections of the grid which must allow the pulmonary arteries and veins, which enter and depart the heart at the sides, must have grid pieces which in the improbable eventuality that the heart must be removed from the cage, facilitate this. At the apical and abapical, or antapical, poles of the heart cage are higher density protective foam-lined ellipsoidal, or oval, apertures, divided in half between the halves of the cage so that these open. The apertures are wide enough to accommodate the off-axis north-south symmetry of the anatomy, and sufficient in area to pass through the blood mainlines attached to the stumps of the cardiac vessels past the diversion servovalves when the cage is opened.

The use of a heart cage as described below in the next section 2b(1)(b)(b6), entitled Heart Cage for the Infrarenal Abdominal Positioning of a Second Heart, and shown in FIG. 24, may itself become bridged by adhesion to the parietal peritoneum, warranting the aggressive use of chemical and material adhesion barriers. The extensive literature describing chemical and film coating means for preventing the formation of adhesions is partially cited above in section 2a, entitled Background. General, the subsection entitled Prevention of Adhesions. The same means will generally also serve as barriers to prevent adverse foreign body reactions.

2b(1)(b)(b7). Metered Compound Vascular Bypass, or Switched-Organ Liver or Heart-Liver Transplantation

Liver-related disease is briefly addressed above under sections 2a. Background of the Invention. General: 2a(3), entitled Remotely Procured Graft Organ Preservation; and below in section 2b(2)(h)(h2), entitled The ‘Tuned’ Shunt in the Relief of Portal Venous Hypertension. Liver transplantation is made necessary by end-stage liver disease, typically cirrhosis or Budd-Chiari syndrome, or acute liver failure, which posing resistance to the free entry of blood into the liver, increases the portal hypertension. To this extent, pulmonary hypertension is often analogous, in that the condition is due to resistance to inflow into the lungs. Heart-liver transplantation is addressed above in section 2a, entitled Background of the Invention. General.

Unless an impaired heart is replaced, defective circulation such as consequent to a Fontan reconstruction or heart failure will inevitably result in damage not only to the liver, but to the kidneys, the lungs, the spleen, indeed, all the tissues of the body (see, for example, Misaka, T., Yoshihisa., A, Ichijo, Y., Ishibashi, S., Matsuda, M., and 10 others 2023. “Prognostic Significance of Spleen Shear Wave Elastography and Dispersion in Patients with Heart Failure: The Crucial Role of Cardio-splenic Axis,” Clinical Research in Cardiology (Darmstadt, Germany) 112(7):942-953; Ohara, H., Yoshihisa, A., Ishibashi, S., Matsuda, M., Yamadera, Y., and 8 others 2023. “Hepatic Venous Stasis Index Reflects Hepatic Congestion and Predicts Adverse Outcomes in Patients with Heart Failure,” Online, Journal of the American Heart Association (Oxford, England)) 20:12(12):e029857; Ziccardi, M. R., Pendela. V. S., and Singhal, M. 2023. “Cardiac Cirrhosis,” Online, Treasure Island, Florida: StatPearls Publishing Co.; El Hadi, H, Di Vincenzo, A., Vettor, R., and Rossato, M. M. 2020. “Relationship between Heart Disease and Liver Disease: A Two-way Street,” Online, MDPI [Multidisciplinary Digital Publishing Institute] Cells (Basel, Switzerland) 9(3):567).

With patients indulgent toward the overuse of alcohol, acetaminophen, or other drug injurious to the liver such abuse sustained over a significant period will precipitate end stage liver failure (see, for example, Sharma, A. and Nagalli, S. 2023. “Chronic Liver Disease,” Online, Treasure Island, Florida: StatPearls Publishing Co.; Philips, C. A. and Kedarisetty, C. K. 2023. “Palliative Care for Patients with End-stage Liver Disease,” Online, Journal of Clinical and Experimental Hepatology (Gurgaon, India): 13(2):319-328). The resilience and regenerative capacity of the liver also mean that no part of it should be resected to make space for the heterotopic positioning of a graft organ: the liver regenerates to its original size and shape and does so rather quickly. To create the room for positioning of a graft organ in the lower abdomen, no more a removal from the small intestine of a foot or foot and a half should be necessary.

The enjoinder to exercise unqualified attention to hepatic artery reconstruction to include an anomalous supernumerary or accessory hepatic artery upon completing a liver transplant is no less applicable to a liver transplant performed using the compound metered vascular bypass method. “Hepatic artery reconstruction is the most valuable step in correcting graft and recipient survival after liver transplantation,” Akbulut. S., Kutluturk, K., and Yilmaz, S. 2021. “Hepatic Artery Reconstruction Technique in Liver Transplantation: Experience with 3,000 Cases,” Online, Hepatobiliary Surgery and Nutrition (Hong Kong, China) 10(2):281-283)(see also Zhang, R., Zhang, H. Z., Han, T., Wei, Z. G., Shi, Z. Y., and Xu, J. 2023. “Effect of Accessory Hepatic Artery Reconstruction on Prognosis in Orthotopic Liver Transplantation: A Single Center Experience,” Online, BioMed Central Surgery (London, England) 19:23(1):138; Otan, E., Akbulut, S., and Yilmaz, S. 2021. “How to Reduce and Manage Hepatic Arterial Complications in Living and Deceased Donor Liver Transplantations,” Online, Hepatobiliary Surgery and Nutrition (Hong Kong. China) 10(5):731-733).

Defective circulation over an extended period is injurious to the liver, so that the need for heart-liver transplantation is often indicated. When applicable, if not correctible through optimization of the Fontan pathway and physiology, liver disease associated with Fontan circulation or any form of pronounced cardiac impairment is likely to necessitate a heart or a combined heart-liver if not a heart-liver-kidney transplant (Das, I., Pham, S. M., Perry, D. K., and Croome, K. P. 2024, Op cit., above in section 2a(3), entitled Remotely Procured Graft Organ Preservation; Cavaco, J. L., Ferreira, A. R., Jalles, C. A., Lopes, I. M., Braz, A., Pedro, A. O., and de Lacerda A P. 2023. “Hepatic Coma Secondary to Fontan Conduit Thrombosis,” European Journal of Case Reports in Internal Medicine (Milan, Italy) 10(4):003817; de Lange, C. Möller T. Hebelka H. 2023. “Fontan-associated Liver Disease: Diagnosis, Surveillance, and Management,” Online, Frontiers in Pediatrics (Lausanne, Switzerland) 11:1100514; Inuzuka R. Nii M, Inai K, Shimada E, Shinohara T. and 15 others 2023. “Predictors of Liver Cirrhosis and Hepatocellular Carcinoma among Perioperative Survivors of the Fontan Operation,” British Cardiac Society Heart (London, England) 109(4):276-282; Lee, J. T. C., Sarode, D., and Emamaullee, J. A. 2023. “Review of Pediatric Combined Heart-Liver Transplantation: A Roadmap to Success,” Online, Pediatric Transplantation (Copenhagen, Denmark) 27(8):e14633; Lewis M J. Reardon L C, Aboulhosn J. Haeffele C. Chen S. and 27 others 2023. “Clinical Outcomes of Adult Fontan-associated Liver Disease and Combined Heart-Liver Transplantation, Journal of the American College of Cardiology 81(22):2149-2160; Meyer, Z., Haas, N., Mühlberg. R., Braun, A., Fischer, M., and Mandilaras, G. 2023. “Transient Liver Elastography in the Follow-up of Fontan Patients: Results of a Nation Wide Survey in Germany,” Online, Frontiers in Pediatrics (Lausanne, Switzerland) 30:11:1194641; Tracy, K. M., Matsuoka, L. K., and Alexopoulos, S. P. 2023. “Update on Combined Heart and Liver Transplantation: Evolving Patient Selection, Improving Outcomes, and Outstanding Questions,” Current Opinion in Organ Transplantation (Hagerstown, Maryland) 28(2):104-109; Fernandes, E., da Silva, F. R., Segalote, R., Rocha P T. Mello F. and 14 others 2022. “Combined Heart-Liver-Kidney Transplantation: The First Experience in Latin America,” Online, Arquivos Brasileiros de Cirurgia Digestiva (São Paulo, Brasil) 35: e1668; Frountzas, M., Karampetsou, N., Nikolaou, C., Schizas, D., Tsapralis, D., Avgerinos, D., and Toutouzas, K. 2022. “Combined Heart and Liver Transplantation: An Updated Systematic Review,” Online, Annals of the Royal College of Surgeons of England (London, England) 104(2):88-94; Perez-Gutierrez, A., Siddiqi, U., Kim, G., Rangrass, G., Kacha. A., and 5 others 2022.

“Combined Heart-Liver-Kidney Transplant: The University of Chicago Medicine Experience,” Clinical Transplantation (Copenhagen, Denmark) 36(4):e14586; Zhu, Y. K., Zhou, Y. F., Zhang, T. X., and Yao, Y. X. 2022. “Anesthesia Management of Combined Sequential Heart-Liver Transplantation Using a Caval Clamp without Venovenous Bypass: A Case Report,” Online, Heliyon (London, England) 8(9):e10730; Dillman, J. R., Trout. A. T., Alsaied, T., Gupta, A., and Lubert, A. M. 2020. “Imaging of Fontan-associated Liver Disease,” Pediatric Radiology (New York, New York) 50(11):1528-1541; Gong. T. and Hall, S. 2020. “Considerations and Experience Driving Expansion of Combined Heart-Liver Transplantation,” Current Opinion in Organ Transplantation (Hagerstown, Maryland) 25:496-500; Kogiso, T. and Tokushige, K. 2020. “Fontan-associated Liver Disease and Hepatocellular Carcinoma in Adults,” Online, Nature Scientific Reports (London, England) 10(1):21742; Rizvi, S. S. A., Challapalli, J., Maynes, E. J., Weber, M. P., Choi, J. H., and 6 others 2020. “Indications and Outcomes of Combined Heart-Liver Transplant: A Systematic Review and Met-analysis,” Transplantation Reviews (Orlando, Florida/Philadelphia, Pennsylvania) 34(2):100517; Tellez, L., Rodriguez de Santiago, E., Minguez, B., Payance. A., Clemente, A., and 19 others 2020. “Prevalence, Features, and Predictive Factors of Liver Nodules in Fontan Surgery Patients,” Journal of Hepatology (Copenhagen, Denmark) 72(4):702-710; Ebong, I. A., Sayer, G., Kim, G., Jeevanandam, V., Baker, T., and 6 others 2019. “Simultaneous Heart, Liver and Kidney Transplantation: A Viable Option for Heart Failure Patients with Multiorgan Failure,” Online, Journal of Heart and Lung Transplantation (New York, New York) 38(9):997-999; Gordon-Walker, T. T., Bove, K., and Veldtman, G. 2019. “Fontan-associated Liver Disease: A Review,” Journal of Cardiology. (Nihon Shinzōbyō Gakkai)(Tokyo, Japan) 74(3):223-232; Lebray, P. and Varnous, S. 2019. “Combined Heart and Liver Transplantation: State of Knowledge and Outlooks,” Clinics and Research in Hepatology and Gastroenterology (Paris, France) 43(2):123-130; Bryant, R. 3rd, Rizwan, R., Zafar, F., Shah, S. A., Chin, C., Tweddell, J. S., and Morales, D. L. 2018. Contemporary Outcomes of Combined Heart-Liver Transplant in Patients with Congenital Heart Disease,” Online, Transplantation (Hagerstown, Maryland): 102(2):e67-e73; D Souza B A, Fuller S. Gleason L P. Hornsby N. Wald J. and 6 others 2017. “Single-center Outcomes of Combined Heart and Liver Transplantation in the Failing Fontan,” Clinical Transplantation (Copenhagen, Denmark) 31(3); Beal, E. W., Mumtaz, K., Hayes, D. Jr., Whitson, B. A., and Black, S. M. 2016. “Combined Heart-Liver Transplantation: Indications, Outcomes and Current Experience,” Online, Transplantation Reviews (Orlando, Florida/Philadelphia, Pennsylvania) 30(4):261-268; Greenway, S. C., Crossland, D. S., Hudson, M., Martin, S. R., Myers R P, and 3 others 2016. “Fontan-associated Liver Disease: Implications for Heart Transplantation,” Journal of Heart and Lung Transplantation (New York, New York) 35(1):26-33; Careddu, L., Zanfi, C., Pantaleo, A., Loforte, A., Ercolani, G., and 5 others 2015. “Combined Heart-Liver Transplantation: A Single-center Experience,” Transplant International (Lausanne, Switzerland) 28(7):828-834).

Liver transplantation using the metered compound vascular bypass, or the switched-organ method eliminates blood loss and the need for blood replenishment as major concerns and obstacles to a successful outcome, and no less important, allows an interruption in circulation with ischemia-reperfusion injury to be eliminated when the donor can be started on life support before dying. Liver transplantation using side-entry diversion jacket vascular valves for sudden or vascular servovalves for metered switching from the diseased liver of the recipient to that of the donor reduces if not eliminates the massive loss of blood due to hemorrhage and high transfusion volumes for which deceased donor orthotopic liver transplantation is known and eliminates ischemic time and cold preservation and reperfusion injury.

Due to its involvement in metabolism, transplantation of the liver in an adult is by means of the metered switch technique as immune tolerance-inducing even without drugs or cellular therapy and therefore less likely to provoke a troublesome rejection reaction and the need for intensive immunosuppression. Application of the sudden switch technique is generally reserved for neonates with an undeveloped immune system.

These impediments are not the exception but rather common (see, for example, Bezinover, D., Dirkmann, D., Findlay, J., Guta. C., Hartmann, M., and 9 others 2018. “Perioperative Coagulation Management in Liver Transplant Recipients,” Transplantation 102(4):578-592; Cleland. S., Corredor, C., Ye, J. J., Srinivas. C., and Mccluskey, S. A. 2016. “Massive Haemorrhage in Liver Transplantation: Consequences, Prediction and Management,” Online, World Journal of Transplantation (Pleasanton, California) 6(2):291-305; Hartmann, M., Szalai, C., and Saner, F. H. 2016. “Hemostasis in Liver Transplantation: Pathophysiology, Monitoring, and Treatment,” Online, World Journal of Gastroenterology (Pleasanton, California) 22(4):1541-1550; Nacoti, M., Corbella, D., Fazzi, F., Rapido, F., and Bonanomi, E. 2016. “Coagulopathy and Transfusion Therapy in Pediatric Liver Transplantation,” World Journal of Gastroenterology 22(6):2005-2023; Tanaka, K. A., Key, N. S., and Levy, J. H. 2009. “Blood Coagulation: Hemostasis and Thrombin Regulation,” Anesthesia and Analgesia 108(5):1433-1446; Sauer, I. M., Bechstein, W. O. and Neuhaus, P. 2001. “Liver Transplantation,” in Holzheimer, R. G. and Mannick, J. A., Surgical Treatment: Evidence-based and Problem-oriented, Munich, Germany: W. Zuckschwerdt Verlag: Williams, R., Neuberger, J., and Calne. R. Y. 1985. “Current Status of Liver Transplantation,” Indian Journal of Gastroenterology 4(4):265-269; Calne, R. Y. 1978. “Practical Aspects of Liver Transplantation,” Annales Chirurgiae et Gynaecologie (Helsinki, Finland) 67(2):39-48).

Conventional liver transplantation with an intact liver implanted is hampered primarily by problems of bleeding and hepatic artery and portal vein thrombosis. Because it depends upon diversion servovalve-jackets devised to allow continuous blood flow throughout the operation, prevent bleeding, and provide direct paths into the vessels for the release of an anticoagulant or thrombolytic and antimicrobial, both metered and sudden switch transplantation should alleviate these problems (see, for example, Azoulay, D., Feray, C., Lim, C., Salloum, C., Conticchio, M., and 6 other 2022. “A Systematic Review of Auxiliary Liver Transplantation of Small-for-size Grafts in Patients with Chronic Liver Disease,” Online, JHEP [Journal of Hepatology Online] Reports 12(4):100447; Debabneh, Y. and Mousa, O. Y. 2022. “Liver Transplantation,” Online, Treasure Island, Florida: StatPearls Publishing Co.; Li, J., Ren, J., Zhang, J., and Meng. X. 2022. “A Modified Kidney-sparing Portal Vein Arterialization Model of Heterotopic Auxiliary Liver Transplantation Increases Liver IL-6, TNF-alpha, and HGF [hepatocyte growth factor] Levels and Enhances Liver Regeneration: An Animal Model,” Online, BioMed Central Surgery 22:281; Kim, J. S., Kim, K. W., Choi, S. H., Jeong, S. Y., Kwon, J. H., Song, G. W., and Lee, S. G. 2019. “Hepatic Artery Occlusion after Liver Transplantation in Patients with Doppler Ultrasound Abnormality: Increasing Sensitivity of Contrast-enhanced Ultrasound Diagnosis,” Korean Journal of Radiology 20(3):459-468; Alobaidi, R., Anton, N., Cave, D., Moez, E. K., and Joffe, A. R. 2018. “Predicting Early Outcomes of Liver Transplantation in Young Children: The EARLY [short term sequelae] Study,” World Journal of Hepatology 10(1):62-72; Borst, A. J., Sudan, D. L., Wang, L. A., Neuss, M. J., Rothman, J. A., and Ortel. T. L. 2018. “Bleeding and Thrombotic Complications of Pediatric Liver Transplant,” Pediatric Blood and Cancer 65(5):e26955; De Pietri, L., Montalti. R., Nicolini, D., Troisi. R. I., Moccheggiani, F., and Vivarelli, M. 2018. “Perioperative Thromboprophylaxis in Liver Transplant Patients,” World Journal of Gastroenterology 24(27):2931-2948; Echeverri, G. J., Caicedo, L. A., Delgado, A., Thomas, L. S., Garcia, V. H., and 4 others 2018. “Aspergillus Hepatic Artery Thrombosis in Liver Transplantation,” Medical Mycology Case Reports 22:27-29; Jung, D. H., Park, C. S., Ha, T. Y., Song, G. W., Park, G. C., Cho, Y. P., and Lee, S. G. 2018. “Placement of an Aortohepatic Conduit as an Alternative to Standard Arterial Anastomosis in Liver Transplantation,” Annals of Transplantation 23: 61-65; Mehta, N. N., Mangla, V.2. Varma. V., Lalwani, S., Mehrotra. S., Chawla. D., and Nundy, S. 2018. “Minimizing Hepatic Artery Thrombosis and Establishing Safety of Grafts with Dual Arteries in Living Donor Liver Transplantation,” Transplantation Proceedings 50(5):1378-1385; Nacoti, M., Ruggeri, G. M., Colombo, G., Bonanomi, E., and Lussana, F. 2018. “Thrombosis Prophylaxis in Pediatric Liver Transplantation: A Systematic Review,” World Journal of Hepatology 10(10):752-760; Olivo, R., Guarrera, J. V., and Pyrsopoulos, N. T. 2018. “Liver Transplantation for Acute Liver Failure” Clinics in Liver Disease 22(2):409-417; Samoylova, M. L., Barbas, A. S., Ravindra, K. V., Sudan, D. L., and Vikraman, D. S. 2018. “Graft Loss Due to Thrombosis of an Aortic Conduit in a Pediatric Multivisceral Transplant Recipient,” Transplantation Direct 4(12):e407; Schwabe, R. F. and Luedde. T. 2018, Op cit. above in section 2b(1)(b7); Herrero, A., Souche, R., Joly, E., Boisset, G., Habibeh, H., and 7 others 2017. “Early Hepatic Artery Thrombosis after Liver Transplantation: What is the Impact of the Arterial Reconstruction Type?,” World Journal of Surgery 41(8):2101-2110; Sugi, M. D., Albadawi, H., Knuttinen, G., Naidu, S. G., Mathur, A. K., Moss, A. A., and Oklu. R. 2017. “Transplant Artery Thrombosis and Outcomes,” Cardiovascular Diagnosis and Therapy 7 (Supplement 3):S219-S227; Meek, J. C., McDougal, J. S., Borja-Cacho, D., and Meek, M. E. 2017. “Use of a Mechanical Thrombectomy Device to Treat Early Hepatic Artery Thrombosis after Orthotopic Liver Transplant,” Radiology Case Reports 13(2):522-526; Jadlowiek, C. C. and Taner, T. 2016. “Liver Transplantation: Current Status and Challenges,” Online, World Journal of Gastroenterology 22(18):4438-4445; Piardi. T., Lhuaire, M., Bruno, O., Memeo. R., Pessaux, P., Kianmanesh, R., and Sommacale, D. 2016. “Vascular Complications following Liver Transplantation: A Literature Review of Advances in 2015,” World Journal of Hepatology 8(1):36-57; Abdelaziz, O. and Attia, H. 2016. “Doppler Ultrasonography in Living Donor Liver Transplantation Recipients: Intra- and Post-operative Vascular Complications,” World Journal of Gastroenterology 22(27):6145-6172; Ma. L., Chen, K., Lu, Q., Ling. W., and Luo, Y. 2016. “Case Report of Hepatic Artery Dissection Secondary to Hepatic Artery Pseudoaneurysm after Living Donor Liver Transplantation,” Online, BioMed Central Gastroenterology 16:44; Ma. L., Lu, Q., and Luo Y. 2016. “Vascular Complications after Adult Living Donor Liver Transplantation: Evaluation with Ultrasonography,” World Journal of Gastroenterology 22(4):1617-1626; Ren, X., Guan, J., Gao, N., Niu. H., and Tang J. 2016. “Evaluation of Pediatric Liver Transplantation-Related Artery Complications Using Intra-Operative Multi-Parameter Ultrasonography,” Medical Science Monitor 22:4495-4502; Yousefzai. R., Trivedi, S., Jain, R., Cheema, O. M., Crouch, J. D., Thohan, V., and Khandheria, B. K. 2015, Op cit.; Chen, J., Weinstein, J., Black, S., Spain, J., Brady, P. S., and Dowell, J. D. 2014. “Surgical and Endovascular Treatment of Hepatic Arterial Complications Following Liver Transplant,” Clinical Transplantation 28(12):1305-1312; Khan, A., Park, P., Oberholzer, J. 1, Tzvetanov. I., Garcia Roca, R., and 3 others 2014. “Concurrent Hepatic Artery and Portal Vein Thrombosis after Orthotopic Liver Transplantation with Preserved Allografts,” Case Reports in Transplantation 2014:384295; Bootun, R. 2013. “Effects of Immunosuppressant Therapy on Wound Healing,” International Wound Journal 10(1):98-104; Akun, E., Yaprak, O., Killi, R., Balci, N. C., Tokat, Y., and Yuzer, Y. 2012. “Vascular Complications in Hepatic Transplantation: Single-center Experience in 14 Years,” Transplantation Proceedings 44(5):1368-1372; Sanchez, S. E., Javid, P. J., Lao. O. B., Dick, A. A., Perkins, J. D., and 4 others 2012. “Hepatic Artery Thrombosis and Liver Malignancy in Pediatric Liver Transplantation,” Journal of Pediatric Surgery 47(6):1255-1260; Willems. M. C., van der Vliet, J. A., de Man, B. M., van der Laak, J. A., Lomme, R. M., and Hendriks, T. 2010. “Persistent Effects of Everolimus on Strength of Experimental Wounds in Intestine and Fascia,” Wound Repair and Regeneration 18(1):98-104; Haque, M., Schumacher, P. A., Harris, A., Scudamore, C. H., Steinbrecher, U. P., and 4 others 2009. “Late Acute Celiac and Hepatic Artery Thrombosis with Portal Vein Thrombosis Resulting in Hepatic Infarction 12 Years Post Orthotopic Liver Transplantation,” Annals of Hepatology 8(4):396-399; Testa, G., Malago', M., Porubsky, M., Marinov, M., Sankary, H., and 3 others 2006. “Hilar Early Division of the Hepatic Duct in Living Donor Right Hepatectomy: The Probe-and-Clamp Technique,” Liver Transplantation 12(9):1337-1341, Rajan, D. K., Stavropoulos, S. W., and Shlansky-Goldberg, R. D. 2004. “Management of Transplant Renal Artery Stenosis,” Seminars in Interventional Radiology 21(4):259-269; Pinna, A. D., Smith, C. V., Furukawa, H., Starzl. T. E., and Fung, J. J. 1996. “Urgent Revascularization of Liver Allografts after Early Hepatic Artery Thrombosis,” Transplantation 62(11):1584-1587; Northover, J. M., Hickman, R., Watson, R. G., and Terblanche, J. 1985. “Healing of the Bile Duct Anastomosis after Transverse Choledochotomy or Transplantation of the Liver in the Pig,” Surgery, Gynecology, and Obstetrics 160(1):33-36; Tzakis, A. G., Gordon, R. D., Shaw, B. W. Jr., Iwatsuki, S., and Starzl. T. E. 1985. “Clinical Presentation of Hepatic Artery Thrombosis after Liver Transplantation in the Cyclosporine Era,” Transplantation 40(6):667-671; Axthelm, S. C., Porter, J. M., Strickland, S., and Baur, G. M. 1979. “Antigenicity of Venous Allografts,” Annals of Surgery (Philadelphia, Pennsylvania) 189(3):290-293; Perloff. L. J., Rowlands, D. T., and Barker, C. F. 1977. “Studies of the Modified Venous Allograft,” Annals of Surgery (Philadelphia, Pennsylvania) 186(2):227-232; Perloff, L. J., Rowlands, D. T. Jr., and Barker, C. F. 1976. “The Antigenicity of Venous Allografts,” Transactions of the American Society for Artificial Internal Organs 22:234-241).

When possible, the nonanomalous hepatic artery and if pertinent, the portal vein of the donor are taken with the liver. Provided source and destination stumps are present, a relatively frequent occurrence—the need to reconstruct an anomalous or stenosed hepatic artery intraoperatively, extending operative and anesthetic duration (see, for example, Kim, J. S., Kim, K. W., Choi, S. H., Jeong. S. Y., Kwon, J. H., Song, G. W., and Lee, S. G. 2019. Op cit. above in this section; Mourad, M. M., Liossis, C., Gunson, B. K., Mergental. H., Isaac, J., and 4 others 2014. “Etiology and Management of Hepatic Artery Thrombosis after Adult Liver Transplantation,” Liver Transplantation 20(6):713-723; Sugi, M. D., Albadawi, H., Knuttinen, G., Naidu, S. G., Mathur, A. K., Moss. A. A., and Oklu, R. 2017. “Transplant Artery Thrombosis and Outcomes,” Cardiovascular Diagnosis and Therapy 7 (Supplement 3):S219-S227; Fouzas, I., Sklavos, A., Bismpa, K., Paxiadakis, I., Antoniadis. N., and 12 others 2012. “Hepatic Artery Thrombosis after Orthotopic Liver Transplantation: 3 Patients with Collateral Formation and Conservative Treatment,” Transplantation Proceedings 44(9):2741-2744; Park, Y. S., Kim, K. W., Lee, S., Lee, J., Jung, D. H., and 7 others 2011. “Hepatic Arterial Stenosis Assessed with Doppler US after Liver Transplantation: Frequent False-positive Diagnoses with Tardus Parvus Waveform and Value of Adding Optimal Peak Systolic Velocity Cutoff,” Radiology 260(3):884-891; Sidhu, P. S., Ellis, S. M., Karani, J. B., and Ryan, S. M. 2002. “Hepatic Artery Stenosis following Liver Transplantation: Significance of the Tardus Parvus Waveform and the Role Mmicrobubble Contrast Media in the Detection of a Focal Stenosis,” Clinical Radiology 57(9):789-799; Merion, R. M., Burtch, G. D., Ham, J. M., Turcotte, J. G., and Campbell, D. A. 1989. “The Hepatic Artery in Liver Transplantation,” Transplantation 48(3):438-443)—can be averted by bypassing these with synthetic tubing, usually a catheter, attached to the stumps by basic ductus side-entry jackets with accessory channels to allow drugs such as an anticoagulant or antimicrobial to be directly released into the bypass.

Such bypasses are described and illustrated in U.S. Pat. No. 11,759,186, entitled Ductus Side-entry Jackets and Prosthetic Disorder Response Systems. Such a jacket with shunt on the aorta can prevent or replace a stenosed celiac artery (Sun, M., Jeffrey, R. B., DiMaio, M. A., and Olcott, E. W. 2016. “Delayed Diagnosis of Celiac Stenosis Causing Hepatic Transplant Ischaemic Necrosis: Diagnosis by Spectral Doppler Findings,” British Journal of Radiology Case Reports 3(1):20150210). The occlusion of finer fluid lines by the accumulation of clot in the circulatory system and crystal in the urinary tract precluded the use of these as bypasses.

Whether in an adult the supra and infrahepatic (subhepatic) vena cavae, hepatic artery, portal vein, and bile duct are anastomosed, the accessory channels supporting all side-entry diversion jacket vascular valves straddle the transections or suture lines, and able to deliver immunosuppressants, anti-inflammatoires, anticoagulants, and antimicrobials, is able to prevent, or if anastomosed, suppress leaks or strictures of the biliary tract, ward off hyperacute rejection, vascular thrombosis, and infection, which abide as permanent and later stenosis of the biliary tree, all threats following liver transplantation.

On this score, the placement of a surface port with side-entry diversion jacket vascular valves, each with at least one accessory channel led from the surface port to either side of each anastomosis, is advisable in any conventional organ transplant. Unless required due to a circumscribed intrahepatic tumor or nonmetastasized carcinoma such as hepatocellular carcinoma restricted to the liver itself, liver disease so progressed as to require transplantation is not limited to the liver itself: invariably, disease is distributed to the hepatic portal vein and structures related to it as secondary extrahepatic portal vein obstruction with thrombosis and cavernomatous collaterals or as primary portal vein obstruction.

In this circumstance, the donor liver is not removed to be situated amid diseased associated structures; rather the healthy liver-related structures corresponding to those diseased in the recipient are taken with the healthy liver. As with any organ disease, transplantation carries lifelong risks, the need for constant medical vigilance, and should not be considered until more conservative medical treatment has failed. Inclusion of associated structures is by recession in placement of the side-entry diversion jacket vascular valves to the widest caliber of the foregoing ductus, without affecting the anatomy superior to these connections.

Because liver transplantation by sudden or metered switching eliminates ischemia and reperfusion, the need for treatment with hyperbaric oxygen is likewise eliminated. Ischemia and reperfusion damage reduce the odds for a favorable outcome (Panackel. C., Fawaz, M., Jacob, M., and Raja, K. 2023. “Pulmonary Assessment of the Liver Transplant Recipient,” Online, Clinical and Experimental Hepatology 13(5):895-911; Xiao, B., Xia, W., Zhang, J., Liu, B., and Guo, S. 2010. “Prolonged Cold Ischemic Time Results in Increased Acute Rejection in a Rat Allotransplantation Model,” Journal of Surgical Research 164(2):e299-304) and ischemia and reperfusion injury, along with problem bleeding, hyperacute rejection, and infection have been the major challenges to success in liver transplantation. The case for hyperbaric oxygen for both donors and recipients in liver transplantation remains under investigation (Lv. H., Han, C. H., Sun, X. J., and Liu, W. W. 2016. “Application of Hyperbaric Oxygen in Liver Transplantation,” Medical Gas Research 6(4):212-218).

Bleeding at hepatectomy is problematic largely because of the numerous portosystemic collaterals which unlike a surgically constructed portacaval shunt, spontaneously develop to reduce, or decompress, the portal hypertension. While sometimes significantly if not completely remediable (see, for example, Thirapattaraphan, C., Treepongkaruna, S. Ruangwattanapaisam, N. and Sae-Giay, S. 2020. “Congenital Extrahepatic Portosystemic Shunt (Abernethy Malformation) Treated with Surgical Shunt Ligation: A Case Report and Literature Review,” Online, International Journal of Surgery Case Reports 66:4-7; Morikawa, N., Honna, T., Kuroda, T., Kitano, Y., Fuchimoto, Y., Kawashima, N., and Kawasaki, K. 2008. “Resolution of Hepatopulmonary Syndrome after Ligation of a Portosystemic Shunt in a Pediatric Patient with an Abernethy Malformation,” Journal of Pediatric Surgery 43(2):e35-e38), the detection of congenital portosystemic shunts, or the Abernethy malformation, in a neonate likely to suffer from hypoxemia should signal the advisability of dispensing with half-way measures as soon as possible and proceeding directly to a liver transplant before the primary defect has been allowed the opportunity to cause systemic, that is, bodily, global, maldevelopment (see, for example, Bahadori, A., Kuhlmann, B., Debray, D., Franchi-Abella. S., Wacker, J., and 3 others 2022. “Presentation of Congenital Portosystemic Shunts in Children,” Online, Children (Basel, Switzerland) 9(2):243; Bansal, K., Gore, M., and Mittal, S. 2022. “Hepatopulmonary Syndrome,” Online, Treasure Island, Florida: StatPearls Publishing Co.; Goncalves, I., Barros, D., Araujo, M., Machado, A. I., Oliverra, C., and Pinto, L. 2022. “Type II Abernethy Malformation: A Rare Cause of Hepatic Encephalopathy in Adulthood,” Online, European Journal of Case Reports in Internal Medicine 9(1):003145; Kakiuchi, T., Baba, Y., Minematsu, N., Doi. H., and Kumamoto, T. 2022. “Progress of Pulmonary Hypertension and High Manganese Levels through Congenital Portosystemic Shunt Closure,” Online, Clinical Case Reports 10(9):e6318; Laleman, W., Pratiknjo, M., Lauridsen, M. M., Bonne, L., Vanderschueren, E., and 4 others 2022. “Closing Spontaneous Portosystemic Shunts in Cirrhosis: Does It Make Sense? Does It Work? What Does It Take?,” Metabolic Brain Disease November 19 [in press]; Ji. L., Ji. Z., Xiang. D., Qin. Y., and Yang. S. 2022. “Case Report: Rare Abernethy Malformation with Hepatopulmonary Syndrome in a Pediatric Patient” Online, Frontiers in Pediatrics 10:856611; Mukund, A., Choudhury, S. P., Tripathy, T. P., Ananthashayana, V. H., Jagdish, R. K., and 4 others 2022. “Influence of Shunt Occlusion on Liver Volume and Functions in Hyperammonemic Cirrhosis Patients Having Large Porto-systemic Shunts: A Randomized Control Trial,” Hepatology International 17(1):150-158; erratum October 10 [in press]; Ngu. N. L. Y., Saxby, E., Farmer, C. C., Lyon, S. and Le, S. 2022. “Functional Recovery from Refractory Hepatic Encephalopathy following Angiographic Obliteration of a Large, Spontaneous Portal-Umbilical Portosystemic Shunt: A Case Report,” Online, CWIR [cardiovascular and interventional radiology] Endovascular 5(1):45; Singhai. A., Mallik, M., and Jain, P. 2022. “Unmasking Hypoxia in Cirrhosis Patients: Six-minute Walk Test as a Screening Tool for Hepatopulmonary Syndrome,” Online, Advanced Biomedical Research 11:50; Aragon Pinto, C., Iyer, V. N., Albitar, H. A. H., Anderson, A., Caigas, H., and 4 others 2021. “Outcomes of Liver Transplantation in Patients with Hepatopulmonary Syndrome in the Pre and Post-MELD [Model for Endstage Liver Disease] Eras: A Systematic Review,” Respiratory Medicine and Research 80:100852; Kadry Z., Schaefer, E., Krok, K., Faust, A., Stine, J. G., and 3 others 2021. “Excellent Outcomes with Liver Transplantation in Hepatopulmonary Syndrome across Pre-transplant PaO: Spectrum,” Online, JHEP [Journal of Hepatology] Reports 3(5):100351; Kumar, P., Bhatia, M., Garg. A., Jain, S., and Kumar, K. 2021. “Abernethy Malformation: A Comprehensive Review,” Online, Diagnostic and Interventional Radiology 28(1):21-28; Oliver, T. I., Sharma, B., and John, S. 2021. “Portal Hypertension,” Online, Treasure Island, Florida: StatPearls Publishing Co.; Jose, A., Shah, S. A., Anwar, N., Jones, C. R., McCormack, F. X., Sherman, K. E., and Elwing, J. M. 2021. “Predictors of Outcomes following Liver Transplant in Hepatopulmonary Syndrome: An OPTN [Organ Procurement and Transplantation Network] Database Analysis,” Respiratory Medicine 190:106683; Xu. S., Zhang, P., Hu. L., Zhou, W. and Cheng, G. 2021. “Case Report: Clinical Features of Congenital Portosystemic Shunts in the Neonatal Period,” Online, Frontiers in Pediatrics 9:778791; DiPaola, F., Trout. A. T., Walther, A. E., Gupta. A. Sheridan. R., and 6 others 2020. “Congenital Portosystemic Shunts in Children: Associations, Complications, and Outcomes,” Online, Digestive Diseases and Sciences 65(4):1239-1251; Lin, Y., Li, X., Li, S., Ba. H., Wang, H. and Zhu, L. 2020. “Treatment Option for Abernethy Malformation—Two Cases Report and Review of the Literature, Online, Frontiers in Pediatrics 8:497447; Nardelli, S., Riggio, O., Giola, S., Puzzono, M., Pelie. G., and Ridola, L. 2020. “Spontaneous Porto-systemic Shunts in Liver Cirrhosis: Clinical and Therapeutic Aspects,” Online, World Journal of Gastroenterology 26(15): 1726-1732; Praktiknjo. M., Simon-Talero. M., Römer, J., Roccarina, D., Martinez, J., and 39 others 2020. “Total Area of Spontaneous Portosystemic Shunts Independently Predicts Hepatic Encephalopathy and Mortality in Liver Cirrhosis,” Online, Journal of Hepatology 72(6):1140-1150; Baiges, A., Turon, F., Simon-Talero, M., Tasayco. S., Bueno, J. and 43 others 2019. “Congenital Extrahepatic Portosystemic Shunts (Abernethy Malformation): An International Observational Study,” Hepatology 71(2):658-669; Phillips, C. A., Rajesh, S., Augustine. P., Padsalgi. G. and Ahamed, R. 2019. “Portosystemic Shunts and Refractory Hepatic Encephalopathy: Patient Selection and Current Options,” Online, Hepatic Medicine: Evidence and Research 11:23-34; Bandali, M. F., Mirakhur, A., Lee, E. W., Ferris, M. C., Sadler. D. J., Gray, R. R., and Wong, J. K. 2017. “Portal Hypertension: Imaging of Portosystemic Collateral Pathways and Associated Image-guided Therapy,” World Journal of Gastroenterology 23(10):1735-1746; Grilo-Bensusan, I. and Pascasio-Acevedo, J. M. 2016. “Hepatopulmonary Syndrome: What We Know and What We Would Like to Know,” Online, World Journal of Gastroenterology 22(25):5728-5741; Leite, A. F., Mota, A. Jr., Chagas-Neto, F. A., Teixeira, S. R., Elias Junior, J., and Muglia, V. F. 2016. “Acquired Portosystemic Collaterals: Anatomy and Imaging,” Radiologia Brasileira J49(4):251-256; Pillai, A. K., Philips, C. A., Arora, A., Shetty, R., and Kasana, V. 2016. “A Comprehensive Review of Portosystemic Collaterals in Cirrhosis: Historical Aspects, Anatomy, and Classifications,” International Journal of Hepatology 2016:6170243; Pargewar, S. S., Desai, S. N., Rajesh, S., Singh, V. P., Arora, A., and Mukund, A. 2016. “Imaging and Radiological Interventions in Extra-hepatic Portal Vein Obstruction,” World Journal of Radiology 8(6):556-570; Andring, B., Patel, A., Trimmer, C., and Kalva, S. P. 2015. “Portal Hypertension: A Review of Portosystemic Collateral Pathways and Endovascular Interventions,” Clinical Radiology 70(10):1047-1059; Sahu, M. K., Bisol, A. K., Chandler, N. C., Agawala, S. and Chauhan, S. 2015. “Abernethy Syndrome: A Rare Cause of Hypoxemia: A Case Report,” Online, Annals of Pediatric Cardiology 8(1):64-66; Sharma, M. and Rameshbabu, C. S. 2012. “Collateral Pathways in Portal Hypertension,” Journal of Clinical and Experimental Hepatology 2(4):338-352; Vishwakarma, P. 2002. “Collateral Pathways in Portal Hypertension,” at https://www.slideshare.net/mishambbs/collateral-pathways-in-portal-hypertension; “the inability of the liver to metabolize certain vasodilators leads to hyperdynamic splanchnic circulation resulting in increased portal blood flow” (Pillai, A. K., Andring, B., Patel, A., Trimmer, C., and Kalva, S. P. 2015, Op cit.); and the hyperdynamic, dilated, thin walled splanchnic circulation likely present with hypertension much less a cirrhotic liver (Cleland. S., Corredor, C., Ye, J. J., Srinivas. C., and Mccluskey, S. A. 2016, et al. above in this section), and the large size of the in- and outflow vessels, to include the hepatic and hepatic portal veins, inferior vena cava, the hepatic artery, and numerous collaterals, all of which in a conventional transplantation are transected.

Short of liver transplantation, the only effective treatment for the hepatopulmonary syndrome, which likewise causes chronic hypoxemia and dyspnea that adversely affect all of the organs and tissues of the body in advanced liver disease-probably due to a failure of the liver to adequately filter out vasodilators from the blood resulting in intrapulmonary arteriovenous dilatations so that blood passes through the pulmonary vessels too quickly to allow adequate oxygenation (references cited above in this section).

Yet another condition for which the only adequate remedy is a liver transplant is the hepatorenal syndrome (see, for example, Ranasinghe, I. R., Sharma, B., and Bashir, K. 2022. “Hepatorenal Syndrome,” Online, Treasure Island, Florida: StatPearls Publishing Co.; Aparici, C. M., Bains, S. N., Carlson, D., Qian, J., Liou, D., and 7 others 2016. “Recovery of Native Renal Function in Patients with Hepatorenal Syndrome following Combined Liver and Kidney Transplant with Mercaptoacetyltriglycine-3 Renogram: Developing a Methodology,” World Journal of Nuclear Medicine 15(1):44-49; Modi, R. M., Patel, N., Metwally, S. N., and Mumtaz, K. 2016, Op cit., above in section 2a(3), entitled Remotely Procured Graft Organ Preservation).

To avoid much bleeding, when the diseased liver of the recipient is excised, the collaterals are ligated near their distal terminus just short of the incisions to remove the liver. A need for liver transplantation usually arises where portal hypertension, addressed below, has become considerable and damaging to the venous connections as well as what is usually the primary cause for the portal hypertension, the cirrhosed liver. This elevated pressure aggravates the bleeding problem. Using side-entry diversion jackets, the major vessels need not be transected at all or can be clamped and anastomosed while flow is bypassed about the suture line, high pressure extravasation into the side connector during placement due to portal hypertension counteracted by increasing the pressure of water delivery through the water jacket.

The portal vein may also become obstructed due to extrahepatic causes such as thrombosis or cavernoma, which are usually correctible without the need for a liver transplant (see, for example, Achar, S., Dutta, H. K., and Gogoi. R. K. 2017. “Extrahepatic Portal Vein Obstruction in Children: Role of Preoperative Imaging,” Journal of the Indian Association of Pediatric Surgeons 22(3):144-149; Pargewar, S. S., Desai, S. N., Rajesh, S., Singh, V. P., Arora, A., and Mukund, A. 2016. “Imaging and Radiological Interventions in Extra-hepatic Portal Vein Obstruction,” World Journal of Radiology 8(6):556-570; Wani, Z. A., Bhat, R. A., Bhadoria, A. S., and Maiwall, R. 2015. “Extrahepatic Portal Vein Obstruction and Portal Vein Thrombosis in Special Situations: Need for a New Classification,” Saudi Journal of Gastroenterology 21(3):129-138). A direct line from a small port at the body surface can b used to target an anticoagulant and/or other medication directly into the portal vein. If not medically manageable resection is indicated.

Broadly, transplantation of a complete liver from a somatically supported neonate or infant to another infant without the introduction of exogenous alloantibodies or a significant load of microbial antigens inadvertently introduced from another source is by the sudden switch technique. Where this is not so or thrombolysis may be necessary, the use of the metered switch technique allows treatment of this factor throughout the circulatory systems of both donor and recipient as the graft organ is gradually switched from the donor to the recipient.

A basic side-entry jacket on the hepatic artery allows the direct targeting of an anticoagulant or thrombolytic (Nacoti, M., Ruggeri, G. M., Colombo, G., Bonanomi, E., and Lussana, F. 2018. “Thrombosis Prophylaxis in Pediatric Liver Transplantation: A Systematic Review,” World Journal of Hepatology 10(10): 752-760; Patel, Y. A., Henson, J. B., Wilder, J. M., Zheng. J., Chow, S. C., and 3 others 2018. “The Impact of Human Leukocyte Antigen Donor and Recipient Serotyping and Matching on Liver Transplant Graft Failure in Primary Sclerosing Cholangitis, Autoimmune Hepatitis, and Primary Biliary Cholangitis,” Clinical Transplantation 32(10):e13388; Martin, E. F. and Levy, C. 2017. “Timing, Management, and Outcomes of Liver Transplantation in Primary Sclerosing Cholangitis,” Seminars in Liver Disease 37(4):305-313; Shetty, N. S. and Shah, I. 2016. “Neonatal Cholestasis Due to Primary Sclerosing Cholangitis,” Journal of Family Medicine and Primary Care 5(4):863-864; Fosby, B., Næss, S., Hov, J. R., Traherne, J., Boberg, K. M., and 7 others 2014. “HLA [human leukocyte antigen] Variants Related to Primary Sclerosing Cholangitis Influence Rejection after Liver Transplantation,” World Journal of Gastroenterology 20(14):3986-4000; Venkat. V. L., Ranganathan, S., Mazariegos, G. V., Sun, Q., and Sindhi, R. 2014. “Recurrence of Primary Sclerosing Cholangitis in Pediatric Liver Transplant Recipients,” Liver Transplantation 20(6):679-686; Spada, M., Riva, S., Maggiore, G., Cintorino, D., and Gridelli, B. 2009. “Pediatric Liver Transplantation,” World Journal of Gastroenterology 15(6):648-674).

A previously implanted automatic response disorder control system is better able to prevent the rejection or infection of a (partial or lobar) liver graft from a living or somatically maintained older child or adult to an infant (see, for example, Lee, H., Park, K. H., Park, H. S., Ryu, J. H., Lim, J., and 4 others 2017. Human Leukocyte Antigen-C Genotype and Killer Immunoglobulin-like Receptor-Ligand Matching in Korean Living Donor Liver Transplantation. Annals of Laboratory Medicine 37(1):45-52; Seda-Neto, J., Antunes da Fonseca, E., Pugliese, R., Candido, H. L., Benavides, M. R., and 10 others 2016. “Twenty Years of Experience in Pediatric Living Donor Liver Transplantation: Focus on Hepatic Artery Reconstruction, Complications, and Outcomes,” Transplantation 100(5):1066-1072).

Both immunologically immature, sudden switch orthotopic transplantation of a liver from an infant on life support to another infant minimizes the risks of both immune responses, and because blood flow is continuous throughout the transfer, eliminates ischemia-reperfusion injury and minimizes the risk of thrombosis. Exogenous alloantigens if present are dealt with pre- and postprocedurally and/or metered compound vascular bypass, or sudden switched-organ transplantation used. Compared to living donor partial transplantation, the drawbacks in switch technique are the shortage of infant livers, the waiting time until one is found while the baby continues to deteriorate, and the fact that a failing liver results in damage to the heart, kidneys, and all the organs and tissues of the body, as well as the intricacy posed and precision required by the tiny sizes of the ligaments, vessels, and nerves.

Switch technique is primarily intended to connect distinct and accessible blood supply and drainage vessels on intact like organs of the donor and recipient without transection through the parenchyma so that vessels are left open and draining. However, in a living donor liver transplantation, the open surface will become the new outer surface of the liver, so that at the surface, these vessels will no longer conduct blood in any event, making the need to avoid a condition of no-flow as it were through the parenchyma inapplicable. Living donor liver transplantation using switch technique essentially treats the portion harvested as if it were the entire liver: the control of bleeding conventional, with a normal recipient, switch technique is by connection of the left hepatic veins, left bile ducts, portal veins, and hepatic arteries of donated portion and these vessels in the recipient.

These connections made before any incision, total orthotopic liver transplantation using a switch technique is essentially bloodless, with living liver transplantation involving far less bleeding (see, for example, Choi, J. U., Hwang, S., Ahn, C. S., Moon, D. B., Ha. T. Y., and 5 others 2019. “Prolonged Occlusion of the Hepatoduodenal Ligament to Reduce Risk of Bleeding and Tumor Spread during Recipient Hepatectomy for Living Donor Liver Transplantation,” Annals of Hepato-Biliary-Pancreatic Surgery 31):61-64; Yoon, J. U., Byeon, G. J., Park, J. Y., Yoon, S. H., Ryu, J. H., and Ri. H. S. 2018. “Bloodless Living Donor Liver Transplantation: Risk Factors, Outcomes, and Diagnostic Predictors,” Medicine (Baltimore, Maryland) 97(50):e13581; Kayaalp. C., Tolan, K., and Yilmaz, S. 2016. “Hepatoduodenal Ligament Dissection Technique during Recipient Hepatectomy for Liver Transplantation: How I Do It?,” Online, World Journal of Transplantation (Pleasanton, California) 6(2):272-277; Li, W. F., Lin, T. L., Tan, T. J., Alikhanov, R. B., Yong, C. C., and 5 others 2016. “Short-term Total Hepatic Vascular Exclusion in Difficult Caudate Lobe Dissection in Living-donor Liver Transplantation Recipients,” Transplantation Proceedings 48(4):1059-1062; Li, W. F., Lin, T. L., Tan, T. J., Alikhanov, R. B., Yong. C. C., and 5 others 2016. “Short-term Total Hepatic Vascular Exclusion in Difficult Caudate Lobe Dissection in Living-donor Liver Transplantation Recipients,” Transplantation Proceedings 48(4):1059-1062; Gad, E. H., Abdelsamee, M. A., and Kamel, Y. 2016. “Hepatic Arterial and Portal Venous Complications after Adult and Pediatric Living Donor Liver Transplantation, Risk Factors, Management and Outcome (A Retrospective Cohort Study),” Annals of Medicine and Surgery (London, England) 8:28-39; Tannuri, U., Tannuri, A. C., Santos, M. M., and Miyatani, H. T. 2015. “Technique Advance to Avoid Hepatic Venous Outflow Obstruction in Pediatric Living-donor Liver Transplantation,” Pediatric Transplantation 19(3):261-266; Tannuri, A. C., Monteiro, R. F., Santos, M. M., Miyatani, H. T., and Tannuri, U. 2014. “A New Simplified Technique of Arterial Reconstruction in Pediatric Living-donor Liver Transplantation: A Comparison with the Classical Technique,” Journal of Pediatric Surgery 49(10): 1518-1521; Utsunomiya, T. and Shimada, M. 2012. “Modified Hanging Method for Liver Resection,” Journal of Hepato-Biliary-Pancreatic Sciences 19(1): 19-24; Li, P. C., Jeng, L. B., Yang, H. R., Lee. C. C., Poon, K. S., and 7 others 2012. “Hepatic Artery Reconstruction in Living Donor Liver Transplantation: Running Suture under Surgical Loupes by Cardiovascular Surgeons in 180 Recipients,” Transplantation Proceedings 44(2):448-450; Tannuri, U., Santos, M. M., Tannuri, A. C., Gibelli, N. E., Moreira, A., and 9 others 2011. “Which is the Best Technique for Hepatic Venous Reconstruction in Pediatric Living-donor Liver Transplantation? Experience from a Single Center,” Journal of Pediatric Surgery 46(7):1379-1384; Nadalin, S., Bockhom, M., Malagó. M., Valentin-Gamazo, C., Frilling, A., and Broelsch, C. E. 2006. “Living Donor Liver Transplantation,” Hepato Pancreato Biliary (Oxford, England) 8(1):10-21).

With a donor and/or in a recipient with situs inversus totalis, for example, the connections in complete orthotopic transplantation from a deceased donor or in a subtotal (portional) transplant from a living donor are adapted to the anomalous anatomy using kink-resistant bloodlines (see, for example, Edgerton, C. A., Gross, M., Kasi. N., Hewitt, W., Edmondson, S., Rohan, V. S., and Nadig, S. N. 2018. “Mirror, Mirror on the Wall” . . . . Pediatric Liver Transplantation in the Case of Situs Inversus Totalis with a Disrupted Inferior Vena Cava,” Pediatric Transplantation 22(5):e13218; Tabrizian, P., Joseph, T. T., Radkani. P., Cohen, E., and Facciuto, M. 2016. “Liver Transplantation in an Adult Recipient with Situs Inversus Totalis: Case Report and Review of the Literature,” Transplantation Proceedings 48(9):3163-3166; Selvakumar, N., Goyal, N., Nayeem, M., Vohra, S., and Gupta, S. 2016. “Does Situs Inversus Totalis Preclude Liver Donation in Living Donor Liver Transplantation? A Series of 3 Cases from Single Institution,” International Journal of Surgery Case Reports 21:23-25; Sankarankutty, A. K., Cagnolati, D., Kemp. R., Souza, F. F., Teixeira, A. C., and 3 others 2015. “How To Do Liver Transplantation in Situs Inversus Totalis: A Simple Technique,” Australian and New Zealand Journal of Surgery 85(3):187-188; Fernandez Soria, N., Garcia Novoa. M. A., Rivas Polo, J. I., Fernandez Selles, C., de Los Angeles Vazquez, M., Marini Milagros, M., and Gomez Gutierrez, M. 2015. “Orthotopic Liver Transplantation in an Adult with Biliary Atresia, Situs Inversus, and Inferior Cava Vein Absence: A Case Report,” Transplantation Proceedings 47(8):2407-2409; Manzia, T. M., Tariciotti, L., Angelico, R., Toti, L., Anselmo, A., and Tisone, G. 2015. “Side-to-side Caval Anastomosis in a Patient Receiving a Liver Graft from a Marginal Donor with Situs Inversus Totalis,” International Journal of Surgery Case Reports 6C: 222-225; Yankol, Y., Mecit. N., Kanmaz, T., Acarlι, K., and Kalayoglu, M. 2015. “Living Donor Liver Transplantation in an Adult Patient with Situs Inversus Totalis,” Ulusal Cerrahi Dergisi [Turkish Journal of Surgery] 31(4):232-234; Yu. S., Guo, H., Zhang, W., Yu, J., Yan, S., and 3 others 2014. “Orthotopic Liver Transplantation in Situs Inversus Adult from an ABO- incompatible Donor with Situs Inversus,” Online, BioMed Central Gastroenterology 14:46; Deuse. T. and Reitz, B. A. 2009. “Heart-Lung Transplantation in Situs Inversus Totalis,” Annals of Thoracic Surgery 88(3):1002-1003; Pomposelli, J. J., DaCosta, M. A., McPartland, K., and Jenkins, R. L. 2007. “Retroversus Implantation of a Liver Graft: A Novel Approach to the Deceased Donor with Situs Inversus Totalis,” American Journal of Transplantation 7(7):1869-1871).

Where conventional technique results in the stoppage of blood flow through the parenchyma, use of sudden or metered switch technique provides continuity of blood flow and therefore elimination of ischemia-reperfusion injury. When transfer of the donor portion to the recipient can be accomplished so quickly that a condition of no-flow is of negligible duration, the conventional technique is used. The comparative results warrant a clinical trial. As conventional, the diseased native organ discarded, the portion donated becomes the new liver; that is, there is no apposition of anastomosis to unite the donor with the native portion.

The success of a living, partial or lobar, liver or lung transplant is, however, fundamentally promoted when supported with the immediacy of response to any adverse eventuality which only an automatic totally implanted system can provide. It is best accompanied by periprocedural immune tolerance induction, immunosuppressants, anti-inflammatoires, and antimicrobials with the procedure performed laparoscopically. Donor and recipient should be positioned side by side, the piece removed from the donor stitched in place in the recipient immediately, thereby achieving continuity of circulation and minimizing ischemia as best might be accomplished.

A healthy donor usually has no need for a prosthetic disorder response system (see, for example, Weng. L. C., Huang, H. L., Lee, W. C., Tsai, Y. H., Wang. W. S., and Chen, K. H. 2019. “Health-related Quality of Life of Living Liver Donors 1 Year after Donation,” Hepatobiliary Surgery and Nutrition 8(1):1-9; Janik, M. K., Księżopolska, A., Kostrzewa, K., Kobryn, K., Moskwa, M., and 6 others 2019. “Long-term Health-related Quality of Life in Living Liver Donors,” Annals of Transplantation 24:45-51; Butt, Z., DiMartini, A. F., Liu, Q., Simpson, M. A., and Smith, A. R., and 11 others 2018. “Fatigue, Pain, and Other Physical Symptoms of Living Liver Donors in the Adult-to-adult Living Donor Liver Transplantation Cohort Study,” Liver Transplantation 24(9):1221- 1232; Dew. M. A., Butt, Z., Liu, Q., Simpson, M. A., Zee, J., and 13 others 2018. “Prevalence and Predictors of Patient-reported Long-term Mental and Physical Health after Donation in the Adult-to-Adult Living-donor Liver Transplantation Cohort Study,” Transplantation 102(1):105-118; Dew. M. A., Butt, Z., Humar, A., and DiMartini, A. F. 2017. “Long-term Medical and Psychosocial Outcomes in Living Liver Donors,” American Journal of Transplantation 17(4):880-892; Humphreville, V. R., Radosevich, D. M., Humar, A., Payne, W. D., Kandaswamy. R., and 4 others 2016. “Longterm Health-related Quality of Life after Living Liver Donation,” Liver Transplantation 22(1):53-62; Xu, D. W., Long, X. D., and Xia, Q. 2015. “A Review of Life Quality in Living Donors after Liver Transplantation,” International Journal of Clinical and Experimental Medicine 8(1):20-26).

However, where living donor transplantation avoids organ shortage and the maldevelopment which results from a failing liver, it poses the risk of both immune rejection and reperfusion injury, whereas switch technique minimizes if not eliminates the risk of immune rejection and eliminates the risk of ischemia-reperfusion injury (see, for example, Kehar, M., Parekh, R. S., Stunguris, J., De Angelis. M., Van Roestel. K., and 9 others 2019. “Superior Outcomes and Reduced Wait Times in Pediatric Recipients of Living Donor Liver Transplantation,” Transplantation Direct 5(3):e430; Nacoti, M., Ruggeri, G. M., Colombo, G., Bonanomi, E., and Lussana, F. 2018. “Thrombosis Prophylaxis in Pediatric Liver Transplantation: A Systematic Review,” World Journal of Hepatology 10(10):752-760; Sethi, P., Thillai. M., Thankamonyamma, B. S., Mallick, S., Gopalakrishnan, U., and 5 others 2018. “Living Donor Liver Transplantation Using Small-for-Size Grafts: Does Size Really Matter?,” Journal of Clinical and Experimental Hepatology 8(2):125-131; Kasahara, M., Sakamoto, S., and Fukuda, A. 2017. “Pediatric Living-donor Liver transplantation,” Seminars in Pediatric Surgery 26(4):224-232; Cuenca, A. G., Kim, H. B., and Vakili, K. 2017. “Pediatric Liver Transplantation,” Seminars in Pediatric Surgery 26(4):217-223; Salvalaggio, P. R., Seda Neto, J., Alves, J. A., Fonseca, E. A., Cameiro de Albuquerque, L., and 11 others 2016. “Consensus, Dilemmas, and Challenges in Living Donor Liver Transplantation in Latin America,” Transplantation 2016 100(6):1161-1164; Elgendy, H., El Moghazy, W. M., Nafady-Hego. H., and Uemoto, S. 2015. “Outcome of Critically-Ill Children after Living-donor Liver Transplant,” Experimental and Clinical Transplantation 13 Supplement 1:100-107; Terrault, N. A., Stravitz, R. T., Lok, A. S., Everson, G. T., Brown, R. S. Jr., and 7 others 2014. “Hepatitis C Disease Severity in Living Versus Deceased Donor Liver Transplant Recipients: An Extended Observation Study,” Hepatology 59(4):1311-1319; Khan, I., Al-Shaqrani, M. A., Arain, Z. B., Al-Hebbi. H. A., Wali, S. H., and Bassas, A. F. 2009. “One Hundred and Thirty-seven Living Donor Pediatric Liver Transplants at Riyadh Military Hospital. Results and Outlook for Future,” Saudi Medical Journal 30(3):403-408; Broering, D. C., Kim, J. S., Mueller, T., Fischer, L., Ganschow, R., and 9 others 2004. “One Hundred Thirty-two Consecutive Pediatric Liver Transplants without Hospital Mortality: Lessons Learned and Outlook for the Future,” Annals of Surgery (Philadelphia, Pennsylvania) 240(6):1002-1012; Borst, A. J., Sudan, D. L., Wang, L. A., Neuss, M. J., Rothman, J. A., and Ortel, T. L. 2018. “Bleeding and Thrombotic Complications of Pediatric Liver Transplant,” Pediatric Blood and Cancer 65(5):e26955; Seda-Neto, J., Antunes da Fonseca, E., Pugliese. R., Candido, H. L., Benavides, M. R., and 10 others 2016. Op cit. above in this section).

2b(1)(b)(b8). Metered Compound Vascular Bypass, or Switched-Organ Single and Double Kidney Transplantation

A difference in the number of donor and recipient vessels, usually arteries, is relatively common, most often encountered in kidneys, and negotiated with the aid of a confluence conduit as addressed above in section 2a and below in sections 2b(1)(b)(b14), entitled Confluence Conduits: Connection of One or More Bloodlines to Flow through a Common Channel to the Same or a Different Number of Bloodlines with Self-leveling Apportionment of Outflow, shown schematically in FIG. 35A, and 2b(1)(b)(b15), entitled Switchable Lines and Confluence Conduits: Long when Intercorporeal, to Short when Intracorporeal, Both Affording Connection of One or More Bloodlines to the Same or a Different Number of Bloodlines with Self-leveling Apportionment of Outflow. and shown schematically in FIG. 35B.

A heart surgically added other than at the center of junction between in- and outflow of all the blood circulated undergoes disuse atrophy due to chronic underload. The same does not apply to kidneys, often implanted heterotopically as supernumerary in the inguinofemoral region or iliac fossae. Except for patients with defective renal arteries or veins, such as a renal artery with an aneurysm resistant to repair, the growing trend of first removing the affected kidney and replacing the same kidney not as added, but heterotopically by autotransplantation in the inguinofemoral region would best be avoided.

Rather than to remove the kidney interrupting its perfusion, the application of the sudden switch technique to move the kidney directly to the inguinofemoral region without interruption in the flow of blood is to be preferred. If the graft organ has accessory vessels, a confluence conduit as described below in section 2b(1)(b)(b14), entitled Confluence Conduits: Connection of One or More Bloodlines to Flow through a Common Channel to the Same or a Different Number of Bloodlines with Self-leveling Apportionment of Outflow, as shown in FIG. 35A is used.

While autotransplantation eliminates rejection, depending upon how long the kidney remains outside or pending connection to an ex vivo perfusion machine—with which to eliminate a stoppage in perfusion with ischemia requires vascular valves to directly switch the flow of blood to and from the machine in any event-ischemia-reperfusion injury may still be a problem adversely affecting graft durability. Favorable outcome self-appraisals may be premature (see, for example, Laurin, T., Borghese, O., Branchereau, J. Karam, G., Brisard, L., and 5 others 2022. “Single Centre Experience in Open and Endovascular Treatment of Renal Artery Aneurysms,” Annals of Vascular Surgery 79:17-24; Ramouz, A. Hafezi, M., Ali-Hasan-Ali-Saegh, S., Shafiei, S., Dezfouli, S. A., and 4 others 2022. “Renal Artery Repair with Kidney Autotransplantation for Renal Artery Aneurysms,” Online, European Journal of Vascular and Endovascular Surgery 63(5):732-742; Contarini, E., Takagi, K., Kimeni, H. J. A. N., Ijzrmans, J. N. M., and 3 others 2021. “Kidney Autotransplantation [heterotopic resituation] for Renal Artery Aneurysm: Case Series and a Systematic Review,” Online, Annals of Vascular Surgery 77:349.e5-349.e18; Smith, M., Lazar, A., Morrissey, N., and Ratner, L. 2020. “Laparoscopic Nephrectomy with ex Vivo Repair of Aneurysm and Autotransplantation,” Online, Journal of Vascular Surgery. Cases, Innovation, and Techniques (New York, New York) 6(1):24-26; Menegolo, M., Colacchio, E. C., Furian, L, Rigotti, P., Taglialavoro, J., and Piazza, M. 2019. “Ex Vivo Open Reconstruction of the Hilar Renal Artery Aneurysms: A Single-center Experience,” in Tshomba, Y. Baccellieri, D and Chiesa, R. (eds.), Visceral Vessels and Aortic Repair, pages 127-134, Cham, Switzerland: Springer International; Gwon, J. G., Han, D. J., Cho, Y.-P., Kim, Y. H., and Kwon, T.-W. 2018. “Role of Heterotopic Kidney Auto-transplantation for Renal Artery Aneurysms,” Online, Medicine (Baltimore, Maryland) 97(23):e10856; Rana, A A., Dias, B. H., Olakkengil, S., Russell, C., and Bhatachariya, S. 2018. “Laparoscopic-assisted ex Vivo Reconstruction of Renal Artery Aneurysm with Internal Iliac Artery and Auto-transplantation,” Online, Cureus (Palo Alto, California) 10) 11):e3611; Silski, L. S., Henry, M. L., Rajab, A., and Pelletier, R. P. 2017. “Case Report: Treatment of Renal Artery Aneurysms by ex Vivo Renal Artery Aneurysm Repair and [Auto and Allo-] Transplantation,” Transplantation Proceedings (New York, New York) 49(10):2374-2377; Morin, J., Chavent, B. Duprey, A. Albertini, J. N., Favre, J. P., and Barral, X. 2012. “Early and Late Results of Ex Vivo Repair and Autotransplantation in Solitary Kidneys,” European Journal of Vascular and Endovascular Surgery (London, England) 43(6):716-720; Richstone, L. and Kavoussi, L. R. 2007. “Re: The Impact of Ischemia Time during Open Nephron Sparing Surgery on Solitary Kidneys: A Multi-institutional Study: R. H. Thompson, I. Frank, C. M. Lohse, I. R. et al.,” Journal of Urology (Philadelphia, Pennsylvania) 177:471-476,” Journal of Urology (Philadelphia, Pennsylvania). 178(3 Part 1):1119; author reply 1119-1120; Thompson, R. J., Frank, I., Lohse, C. M., Saad, I. R., Fergany, A., and 4 others 2007. “The Impact of Ischemia Time during Open Nephron Sparing Surgery on Solitary Kidneys: A Multi-institutional Study,” Journal of Urology (Philadelphia, Pennsylvania) 177(2):471-476).

Provided it can be accomplished without interrupting perfusion, in situ repair of repair-averse aneurysms of a renal artery is also a better method than removal of the graft organ. However, in situ repair that involves clamping also subjects the kidney to ischemia (see, for example, Bilman, V, Mascia, D., Carta, N., Santoro, A. Saracino, C., Chiesa, R. and Melissano, G. 2022. “Contemporary Outcomes of in Situ Open Surgical Repair of Mid-portion and Distal Renal Artery Aneurysms,” Annals of Vascular Surgery 78:9-18; Robinson, W. P. 3, Bafford, R., Belkin, M., and Menard, M. T. 2011. “Favorable Outcomes with in Situ Techniques for Surgical Repair of Complex Renal Artery Aneurysms,” Journal of Vascular Surgery (New York, New York) 53(3):684-691).

Kidneys with accessory (supernumerary, anomalous; and when polar, aberrant) are usually acceptable for donation, the transplantation of kidneys whether orthotopically or heterotopically differing in the number of like vessels expedited through the use of a confluence conduit as addressed below in sections 2b(1)(b)(b14), entitled Confluence Conduits: Connection of One or More Bloodlines to Flow through a Common Channel to the Same or a Different Number of Bloodlines with Self-leveling Apportionment of Outflow, and shown in FIG. 35A, and section 2b(1)(b)(b15), entitled Switchable Lines and Confluence Conduits: Long when Intercorporeal, to Short when Intracorporeal, Both Affording Connection of One or More Bloodlines to the Same or a Different Number of Bloodlines with Self-leveling Apportionment of Outflow, and shown schematically in FIG. 35B.

These are especially pertinent to kidney transplantation involving accessory arteries and/or placed heterotopically, serving two purposes-facilitating the connection of organs differing in the number of vessels between donor and recipient, and enabling the heterotopic placement of graft organs, the heart in particular, where even though like-vessel connections are adhered to, significant deviations in intracardiac pressures would ensue. If heterotopically placed without the removal of either native kidney, a functioning kidney which presents some common vascular anomaly will usually be acceptable.

The problem is remedied by discounting complete correspondence on vascular connections and instead instituting local connections to one or more nearby large arteries or veins using valving, choking, and the tapping off of excess pressures to allow local connection unaffected by vertical distance and gravity. Kidneys are routinely implanted heterotopically without removal of a native kidney and without problematic intrarenal pressures. The value in a confluence conduit is then facilitating the connection of accessory vessels. Flow into a duplicated vessel of which one is stenosed can only apportion the same total flow volume with the open vessel allotted a larger volume of flow according to the degree of restriction imposed by the other.

However, if transplantation seeks to replace the anomalous with normal structure, it is the reverse situation—that unobstructed duplicated vessels should outflow in the correct volume into the one normal vessel—that has practical application. This leveling factor contributed by an interposed confluence conduit is applicable to the replacement of an anomalous organ such as a univentricular by a heart formed normally, where the vessels uninvolved in the defect are normal, so that flow is properly apportioned between the defective sending and one normal receiving vessel.

Moreover, heterotopic placement using the compound vascular bypass, or switched-organ technique, because it eliminates circulatory interruption, or arrest, eliminates ischemia/reperfusion injury, and the other insults associated with graft organ removal and storage prior to implantation, making the expectation of a favorable outcome all the more likely. More broadly, compound vascular bypass transplantation should substantially improve the outcome of any solid organ transplant, orthotopic heterotopic or heterotopic and liberalize positioning when heterotopic.

If positioned anywhere but in the chest without the benefit of confluence conduits, a transplanted heart would have to be provided with lines to duplicate the connections of its in- and outflow vessels, and to compensate for the increase in distances may necessitate such artificial means as mechanical assistance devices, neuromodulation, inotropes, possibly an artificial pacemaker/cardioverter-defibrillator, and support servovalves and/or servochokes to normalize the internal pressures. Accordingly, a position adjacent to the native heart is best for the addition of a second heart.

In contrast, as the norm of heterotopic placement attests, even though the kidneys also process all the blood in the circulatory system, these may be thought of as ‘offline’ rather than pressure central as is the heart. Kidneys tend to afford latitude in positioning without fine adjustments in pressures and glands are not even transplanted intact but rather minced for injection into skeletal muscle. A less than perfect kidney can be transplanted not to replace but rather to assist an impaired native kidney.

The object in such a doublet is that when a good kidney is unavailable, the ability is created to enlist one less than perfect kidney to assist that native to one side, or if necessary, one more to either side to assist that native on both sides. The blood supply is then split between the two. In an ipsilateral native-donor pair, or doublet, regardless of the relative apportionment of blood between the two, both kidneys working together must present sufficient functionality to process the same volume of blood as would one normal kidney. That is, the more impaired is the native kidney, the more competent must be that added. Hence, bilateral pairs must satisfactorily process the same volume of blood as would two normal kidneys.

Even if one or two impaired kidneys added to the native kidney achieve less than complete sufficiency, the interval between visits to the clinic for dialysis can be extended. Such a less than satisfactory resolution can serve as a bridging strategy until a good kidney becomes available, with the quality of life until then much improved. When meter switch transplanted, a kidney, for example, is spared the insults, strongly consequential for success, of ischemia, cold storage, and reperfusion injury of the conventional procedure. This means that the donor kidney need not be as robust as one able to withstand such significant mistreatment.

Another basic advantage is that with metered compound vascular bypass, or sudden switched-organ transplantation, immune tolerance induction is incorporated into the process whereby the graft organ is continuously transferred from the circulatory system of the donor and into that of the recipient. Absent a specific patient with observable signs, symptoms, and measurable diagnostic indicia, to respond for the veritable infinitude of medical possibilities, responsive adaptations, and patient specific anomalies as to a detailed method in the application of any treatment, to include switch transplantation, is not practically feasible (Anderson, W. D., DeCicco, D., Schwaber, J. S., and Vadigepalli, R. 2017, Op cit. section 2b(1)(b) entitled Metered Compound vascular bypass, or sudden switched-organ Transplantation, and the means for its implementation shown, its detailed application in any practical situation must ultimately and always be left to clinical judgment.

As in any supernumerary transplant, there is no good location to place a graft organ, only gradations in the likelihood and nature of what complications are most probable in a given objectionable position. While not imperative, positioning donor kidneys adjacent to the native kidneys just outside the peritoneal lining among folds of the descending colon, for example, facilitates use of the vascular valves to apportion blood flow to and from either in a continuously variable manner, which function can proceed automatically according to the condition of each as reported to the controller by the sensors.

The valves also allow bypassing either kidney in a connected pair to allow either to be removed or resituated. Conventionally, a graft kidney is situated heterotopically by connection to larger vessels in the lower abdomen, or in the iliac fossa rather than orthotopically, even when either or both native kidneys require removal. Kidneys and glands are more amenable to heterotopy than the other organs. Ordinarily, metered compound vascular bypass, or sudden switched-organ transplantation is initiated with living or life supported donor and recipient positioned side by side. To pass the mainlines directly, an orthotopic like-side to like-side kidney replacement is with the two positioned alongside one another with the kidneys centered on the same counteraxial (perpendicular) line.

Differences in distance from the one kidney to the other due to chirality relatively small, to transplant one good or two somewhat impaired kidneys heterotopically, placing the subjects either head to head or head to toe can be used, with consideration as to whether a replacement or added kidney is a right or left disregarded. Because in switch transplantation anastomosis of the donor to the recipient vessels is optional, the left kidney is not preferred due to the greater length of the renal vein. Unless either of both have a body mass index in excess of nine pounds per square foot, or one table is up against a wall or control console, or lines cross the space so that the pair cannot be walked around, no space intervening between the tables should be necessary for the operator and assistants to have access.

For the heterotopic transplantation of a single kidney, the recipient and a donor on life support will have had the implanted disorder response system placed. A living donor is managed without such a system under local or regional anesthesia. The graft organ remaining in the donor, the renal vein and artery of the donor are each connected to the corresponding vessels in the recipient. The highly elastic bloodlines pass from the graft kidney of the donor to cross over the bodies of both and to the kidney of the recipient to be added or replaced. The process begins with the opening of the vascular valves connecting corresponding recipient and donor vessels.

The valves gradually open so that the volume of blood exchanged between donor and recipient is gradually increased. In this way, the system progressively transfers perfusion of the graft organ from the circulatory system of the donor to that of the recipient. Once the circulatory transfer is completed, the donor kidney is harvested and positioned in the recipient iliac fossa. If the donor kidney is to replace a native kidney, the servovalves will have moved all blood flow from the native to the donor kidney, allowing the native kidney to be removed.

If the donor kidney is to share its perfusion with the recipient kidney as in a double heart transplant, the servovalves are set at about the halfway point to evenly split flow through both, for example, the condition of either kidney determining the setpoint for the apportionment of flow between the two. Shortening the procedure reducing the degree of immune tolerance induction achieved, the needs of a living donor are attended to so that departure even over a period of days will be unnecessary. The blood sweeping over and exchanged between the intrarenal endothelia of donor and recipient kidneys during reciprocal cross-circulation should achieve tolerance induction more efficiently and with higher concentration than blood exchanged between the donor renal and recipient external iliac vessels.

Having been meter switch transplanted, the jackets with accessory channels already in place allow the direct pipeline targeting of drugs to the added kidney or kidneys. Regardless of the location chosen for the transplant, conventional kidney transplantation omits the advantages of switch transplantation in the trauma-free gradual transfer of the graft organ from the donor to the recipient without ischemia-reperfusion injury, and immune tolerance induction integrated into the process of transitioning the graft organ for the transfer, which factors encompass the causes for rejection.

This fundamental advantage applies whether the switch is between donor and recipient renal vessels or those of the donor and the recipient iliac vessels. Kidney-kidney reciprocal cross-circulation is prohibited by a condition of the native kidneys that necessitates their removal, switch transplantation then directly to the recipient external iliac vessels. In any situation where the graft kidney or kidneys would not benefit from or would best avoid connection to the native kidneys, to directly meter switch transplant one good or two somewhat impaired kidneys heterotopically to the iliac vessels without connection to the native kidneys is preferred.

Otherwise, unless druglines (sidelines, accessory channels) are run to each kidney as shown in FIG. 16, U.S. Pat. No. 11,759,186, entitled Ductus Side-entry Jackets and Prosthetic Disorder Response Systems which still necessitates access to both kidneys, the native kidneys remain inaccessible for the automatic direct targeting of drugs by the implant disorder response system. The resolution is to provide jackets in order to allow the direct pipeline targeting of drugs to the native kidneys but not connect these to an added kidney. The metered switch method also allows the direct heterotopic placement of a graft kidney in the lower abdomen or inside the iliac fossa of the pelvic girdle, for example; and depending where the connections to the circulatory system are made, eliminates the need to clamp the iliac vessels or the abdominal aorta and inferior vena cava as a cause of complications.

Connection thus does not, however, allow the pairing of a native with a donor kidney in a doublet arrangement, but if the recipient kidneys are accessed, these can be valved for the direct targeting of drugs or to reduce flow-through vis a vis the graft kidney. With living and/or deceased donors on life support, the physiologically fundamental advantage of avoiding ischemia and reperfusion injury are had. Moreover, the retroperitoneal kidneys less accessible, to connect an additional kidney to larger abdominal or iliac vessels is less intricate and reduces the procedural duration.

Metered compound vascular bypass, or sudden switched-organ transplantation thus is little different from connection to the renal artery and vein of the native kidney to be replaced or paired. The same process is used to connect the iliac artery and vein or the abdominal aorta and inferior vena cava of the recipient to the renal artery and vein of the donor kidney to be transferred to the recipient for positioning in the iliac fossa. Because these vessels in a living donor cannot be removed and would cause needless complications with a donor on life support, these connections cannot be reversed. As always, the process is begun by affixing the diversion valves in donor and recipient, the latter also implanted with the automatic response system.

Connection to the donor renal artery and vein are by interposition of servovalve-connected bloodlines, or mainlines, with associated druglines (sidelines, accessory channels) to allow direct drug targeting. Only once these connections have been made and verified as secure are the valves gradually opened to pass blood flow through the donor kidney or kidneys in accordance with the metered switch method. Once perfusion through the graft kidney has progressed over a duration judged adequate to have achieved a sufficient initial immune tolerance induction, removal, or harvesting of the graft kidney is the same as when the graft organ is taken as a replacement or a backup. The free ends of the graft renal vessels are best end-to-end anastomosed.

Pairing either kidney, or exceptionally when only impaired donor kidneys are available, both kidneys, with a potential replacement provides advantages such as the ability to automatically vary the apportionment of blood between the two in response to the instant condition of either, blood cut off entirely only when the affected kidney must be removed. It warrants emphasis that the object in a double kidney transplant using less than ideal donor kidneys and the support of a fully implanted sensor-driven automatic drug pipeline targeting system is to expand the pool of usable kidneys by providing long-term ambulatory renal sufficiency without the need for dialysis at all or the need therefor much less often.

That is, if the native kidneys are impaired, a good kidney unavailable, and visits to the clinic or other causes make dialysis prohibitive, then another kidney can be added to either, or if both native and donor kidneys are impaired, then each native kidney can be paired with a donor. The added kidneys are positioned within the peritoneum. The ureters of the pair at either side are joined by ureteroureteral anastomosis. In a manner analogous to a double heart transplant, unless connection was to the recipient iliac vessels or the abdominal aorta and inferior vena cava, the native and donor, or supernumerary, kidney or kidneys share the blood supply and drainage of the native kidney or kidneys.

In either case, the vascular valve diversion chutes, with apertures smaller if connection is to the larger more central vessels indicated, adjusted to about a midway setpoint. The mainlines are therefore much smaller in caliber than are the valve-jackets connected to the larger vessels in the recipient. Essential for survival, the benefit of renal or renal-liver transplantation for end stage kidney disease critically outweighs the relatively negligible impact upon renal function of denervation following the operation.

Transplanted kidneys generally reinnervate within a year (see, for example, Kopp. U. C. 2018. Neural Control of Renal Function, San Rafael, California: Morgan and Claypool Life Sciences; Mauriello, A., Rovella, V., Borri, F., Anemona, L., Giannini, E., and 10 others 2017. “Hypertension in Kidney Transplantation is Associated with an Early Renal Nerve Sprouting,” Nephrology. Dialysis, Transplantation 32(6):1053-1060; Booth, L. C., Nishi, E. E., Yao. S. T., Ramchandra, R., Lambert, G. W., Schlaich, M. P., and May. C. N. 2015. “Reinnervation following Catheter-based Radio-frequency Renal Denervation,” Experimental Physiology 100(5):485-490; Booth, L. C., Nishi, E. E., Yao, S. T., Ramchandra. R., Lambert, G. W., Schlaich, M. P., and May, C. N. 2015. “Reinnervation of Renal Afferent and Efferent Nerves at 5.5 and 11 Months after Catheter-based Radiofrequency Renal Denervation in Sheep,” Hypertension (Dallas, Texas) 65(2):393-400; Krum H., Schlaich, M., Whitbourn, R., Sobotka, P. A., Sadowski, J., and 7 others 2009. “Catheter-based Renal Sympathetic Denervation for Resistant Hypertension: A Multicentre Safety and Proof-of-Principle Cohort Study,” Lancet (London, England) 373(9671):1275-1281; DiBona, G. F. 1987. “Renal Innervation and Denervation: Lessons from Renal Transplantation Reconsidered,” Artificial Organs 11(6):457-462).

At least one group maintained that microscopic proof of reinnervation following kidney transplantation in man should not be accepted as indicative of a recovery in nervous function (Hansen, J. M., Abildgaard, U., Fogh-Andersen, N., Kanstrup, I. L., Bratholm, P., Plum, I., and Strandgaard, S. 1994. “The Transplanted Human Kidney Does Not Achieve Functional Reinnervation,” Clinical Science (London, England) 87(1):13-20). However, as attested to by the effectiveness of past transplants, the excretion of sodium and other kidney functions is not disabled following surgical denervation of the graft kidney. Rather, alterations in kidney function following transplantation are attributed to the fact that the native kidney or kidneys of the recipient are remain innervated.

In conventional heterotopic kidney transplantation, innervation of the unremoved native kidneys exert control over the release of kidney function-modulating hormones such as natriuretic peptides transmitted through the blood. However, this results in hypertension that can jeopardize graft kidney survival (see, for example, Schneider, S., Promny, D., Sinnecker, D., Byme, R. A., Müller, A., and 6 others 2015. “Impact of Sympathetic Renal Denervation: A Randomized Study in Patients after Renal Transplantation (ISAR-denerve),” Nephrology, Dialysis, Transplantation (Oxford, England) 30(11):1928-1936)(see also Akumwami, S., Morishita, A., Iradukunda, A., Kobara, H., and Nishiyama, A. 2023. “Possible Organ-protective Effects of Renal Denervation: Insights from Basic Studies,” Hypertension Research (London, England) 46(12):2661-2669; Wilson, A. L., Gandhi, J., Suh, Y., Joshi, G., Smith, N. L., and Khan, S. A. 2020. “Renal Innervation in Resistant Hypertension: A Review of Pathophysiology and Renal Denervation as Potential Treatment,” Current Hypertension Reviews (Saif Zone, Sharjah, United Arab Emirates/San Francisco, California) 16(2):115-127; Agasthi, P., Shipman, J., Arsanjani, R., Ashukem, M., Girardo, M. E., and 4 others 2019. “Renal Denervation for Resistant Hypertension in the Contemporary Era: v A Systematic Review and Meta-analysis,” Online, Nature Research Scientific Reports (London, England) 9(1):6200; Shaw, J. A. and Warren, J. L. 2015. “Resistant Hypertension and Renal Denervation Where to Now?,” Online, Cardiovascular Therapy (London, England) 33(1):9-14; Johns, E. J., Kopp. U. C., and DiBona, G. F. 2011. “Neural Control of Renal Function,” Comprehensive Physiology (Bethesda, Maryland) 1(2):731-767; DiBona, G. F. and Kopp, U. C. 1997. “Neural Control of Renal Function,” Physiological Reviews (Bethesda, Maryland) 77(1):75-197).

With kidneys in a doublet, the inlet valve can be adjusted to direct all blood through either kidney, however, some blood is always allowed a kidney sought to be preserved. Should either or both native or donor kidneys become severely enlarged, repeatedly infected, or cause intractable hypertension as to necessitate removal, a doublet arrangement simplifies the orthotopic replacement of the one by the other as an endoscopic procedure. This arrangement also complements direct pipeline targeting to the kidneys of immunosuppressants and anti-inflammatoires keeping these potentially harmful drugs from the rest of the body.

The release into the venous drainage of a reversal agent and/or magnetic means for removing any residue from continued flow are specified in U.S. Pat. No. 11,013,858, entitled Nonjacketing Side-entry Connectors and Prosthetic Disorder Response Systems, U.S. Pat. No. 11,389,171, entitled Integrated System for the Infixion and Retrieval of Implants, and U.S. Pat. No. 11,759,186, entitled Ductus Side-entry Jackets and Prosthetic Disorder Response Systems. Other drugs of which the direct targeting following a kidney transplant is likely to prove advantageous include antihypertensives and sometimes inevitably, antimicrobials.

Should, or one or both native or transplant kidneys in a pair recover due to the reduction in the volume of blood to be processed and/or to the reversal of a precipitative cause elsewhere in the body, the removal of its partner if a cause for discomfort, for example, will have been facilitated in advance (see, for example, Hryniewiecka, E., Hryniewiecki, T., Różański, J., Pilecki, T., Zagożdżon, R., and 5 others 2020. “Reversal of Stage 5 Chronic Kidney Disease by Aortic Valve Replacement in Kidney Transplant Recipient: A Case Report,” Online, BioMed Central Cardiovascular Disorders (London, England) 2020 20(1):20; Kim, H. and Lee, J. H. 2018. “Emergency Transcatheter Aortic Valve Replacement for a Patient with Decompensated Severe Aortic Stenosis Accompanied by Cardiorenal Syndrome: A Case Report,” Online, BioMed Central Cardiovascular Disorders (London, England) 18(1):55; Aparici, C. M., Bains, S. N., Carlson, D., Qian, J., Liou, D., and 7 others 2016, Op cit., above in section 2b(1)(b)(b7), entitled Metered Compound Vascular Bypass, or Switched-organ Liver or Heart-Liver Transplantation; Francis, J. M., Palmer, M. R., Donohoe, K., Curry, M., Johnson, S. R., and 5 others 2012. “Evaluation of Native Kidney Recovery after Simultaneous Liver-Kidney Transplantation” Transplantation 93(5):530-535; Levitsky, J., Baker, T., Ahya, S. N., Levin, M. L., Friedewald, J., and 8 others 2012. “Outcomes and Native Renal Recovery following Simultaneous Liver-kidney Transplantation,” American Journal of Transplantation 12(11):2949-2957; Hidalgo, P., Jimenez, T., Blanca, L., Cobelo, C., Burgos, D., Garcia, I., and Hernandez, D. 2010. “Recovery of Native Renal Function after Kidney Transplantation,” Transplantation Proceedings 42(8):3137-3140; Descoeudres, B., Giannini, O., Aschwanden, M., Eugster, T., Hopfer, H., and 3 others 2009. “Silent Recovery of Native Kidney Function after Transplantation in a Patient with Membranous Nephropathy,” Nephrology, Dialysis, Transplantation 24(4):1345-1349).

Otherwise, this can continue over the 15-20 years living donor graft kidneys are likely to do well with both kidneys on the one or either side receiving and discharging half the normal volume of blood, or until the ipsilateral native kidney fails, whereupon the valves are adjusted to bypass the native kidney and pass all or nearly all blood flow to the donor kidney, allowing the native kidney to be removed. If the donor kidney fails, the valves are adjusted to cut off the donor kidney and pass all blood flow to the native kidney, allowing the donor kidney to be removed, such revisions usually possible endoscopically.

Following a metered switch kidney transplant, if the time for a retransplantation is approaching and the native kidneys, having had this assistance and immediate medical support by the automatic response system show signs of recovery, so that it is determined that the restored native kidneys would best process the full normal volume of blood, then rather than to proceed with a retransplantation, the donor kidney or kidneys are disconnected from the native kidneys and removed.

This can consist of reconstituting the anatomical supply and drainage of the native kidneys by removal of the bloodlines, or mainlines; however, if the conditions that resulted in deterioration of the native kidneys in the first place remains, then the valve-jackets with accessory channels or druglines are best left in place to allow continued automatic drug targeting by the implant disorder response system. This also facilitates a switch retransplantation should such need eventuate.

The implantation of kidneys with accessory, that is multiple, or supernumerary, renal arteries-which occur in almost one third of the population-usually do little to interfere with favorable outcomes. The implantation of or the replacement with a kidney with accessory arteries is considerably facilitated using a confluence conduit such as that shown in FIG. 35B and addressed below in section 2b(1)(b)(b14), entitled Confluence Conduits: Connection of One or More Bloodlines to Flow through a Common Channel to the Same or a Different Number of Bloodlines with Self-leveling Apportionment of Outflow.

Supernumerary vessels as a factor in kidney transplantation is addressed above in sections 2a, entitled, Background of the Invention General; as well as in the present section, entitled 2b(1)(b)(b8) Metered Compound Vascular Bypass, or Switched-organ, Single and Double Kidney Transplantation; and below in sections 2b(1)(b)(b13), entitled Bloodline and Drugline Switch Devices: Long when Intercorporeal to Short when Intracorporeal to Accommodate Growth; 2b(1)(b)(b15), entitled Switchable Lines and Confluence Conduits: Long when Intercorporeal, to Short when Intracorporeal, Both Affording Connection of One or More Bloodlines to the Same or a Different Number of Bloodlines with Self-leveling Apportionment of Outflow; 2b(1)(b)(b16), entitled Direct Aortocaval Connection: Unified Entry Arterial and Exit Venous Confluence Conduits for the Heterotopic Connection of a Graft Heart in the Lower Abdomen; and section 6, entitled Description of the Preferred Embodiments of the Invention,

Reports of complications following the implantation of a kidney with anomalous such as accessory or supernumerary vessels are relatively uncommon (Choudhary, D., Vijayvergiya, R., Kishore, K., Subramani, V. N., Banoth, M., and 11 others 2024. “Vascular Reconstruction of Multiple Renal Arteries—A Risk Factor for Transplant Renal Artery Stenosis: Insight from a Matched Case-control Study,” Transplant International (Lausanne, Switzerland) 37:13298; Cherchi V, Baccarani U, Ventin M, Pravisani R, Puggioni A, and 6 others 2022. “Current Practice with Grafts with Multiple Renal Arteries in Kidney Transplantation: Role of the Methylene Blue in the Lower Pole,” Online, Acta Biomedica (Fidenza Parma, Italy) 93(1):e2022006; Giron-Luque, F., Baez-Suarez, Y., Garcia-Lopez, A., and Patino-Jaramillo, N. 2022. “Safety and Intraoperative Results in Live Kidney Donors with Vascular Multiplicity after Hand-assisted Laparoscopy Living Donor Nephrectomy,” Online, Research and Reports in Urology (Macclesfield, England) 14:23-31; Tabbara, M. M., Guerra, G., Riella, J., Abreu, P., Alvarez. A., and 6 others 2022. “Creating a Single Inflow Orifice from Living Donor Kidney Allografts with Multiple Renal Arteries, Online, Transplant International (Lausanne, Switzerland) 35:10212; Zorgdrager, M., Krikke, C., Hofker, S. H., Leuvenink, H. G., and Pol, R. A. 2016. “Multiple Renal Arteries in Kidney Transplantation: A Systematic Review and Meta-analysis,” Annals of Transplantation (Smithtown, New York) 21:469-478; Paragi, P. R., Klaassen, Z., Fletcher, H. S., Tichauer, M., Chamberlain, R. S., and 3 others 2011. “Vascular Constraints in Laparoscopic Renal Allograft: Comparative Analysis of Multiple and Single Renal Arteries in 976 Laparoscopic Donor Nephrectomies,” World Journal of Surgery (Hoboken, New Jersey) 35(9):2159-2166; Carter, J. T., Freise, C. E., McTaggart, R. A., Mahanty, H. D., Kang, S. M., and 4 others 2005. “Laparoscopic Procurement of Kidneys with Multiple Renal Arteries is Associated with Increased Ureteral Complications in the Recipient,” Online, American Journal of Transplantation (New York, New York) 5(6):1312-1318).

The literature mostly favorable to the use of kidneys with multiple vessels is large. Ureteral complications have been cited as a potential problem but anomalous vessels otherwise seldom result in adverse outcomes. Supernumerary vessels do, however, increase the procedural duration and tend to prompt allograft renal artery stenosis (see, for example, Schmidt, J., Peters, R., Mang, J., Ralla, B., Moldovan, D. E., and 5 others 2024. “Retrospective Analysis of the Perioperative Outcome in Living Donor Kidney Transplantation with Multiple Renal Arteries: Does Accessory Vessel Ligation Affect the Outcome?,” Online, World Journal of Urology (New York, New York) 42(1):161; Eren, E., Tokaç, M., Uslu, B., Şahin, T., Vartanoğlu Aktokmakyan, T., and Dinçkan, A. 2023.

Is the Presence of 2 Renal Allograft Arteries Associated with Adverse Outcomes in Live Donor Kidney Transplantation? Online, Eurasian Journal of Medicine (Istanbul, Turkey) 55(1):74-77; Şahin, S., Özdemir, O., Ekşi, M., Evren, i., Karadağ, S., Arikan, Y., and Taşçι, A.i. 2023. “The Effect of Single or Multiple Arteries in the Donor Kidney on Renal Transplant Surgical Outcomes,” Irish Journal of Medical Science (Dublin, Ireland) 192(2):929-934; Farahzadi. A., Mahmoodzadeh, H., and Gopal, N. A., 2022. “A Solitary Precaval Right Renal Artery: A Case Report,” Online, Clinical Case Reports 10(6):e05866; Giron-Luque, F. Baez-Suarez, Y., Garcia-Lopez, A. and Patino-Jamiillo, N. 2022. “Safety and Intraoperative Results in Live Kidney Donors with Vascular Multiplicity after Hand-assisted Laparoscopy Living Donor Nephrectomy,” Online, Research and Reports in Urology 14:23-31; Kim, J., Lee, J. M., Cho. S. G., and Hong, J. U. 2022. “Ectopic Vascularization of the Right Kidney Arising from Contralateral Common Iliac Artery,” Surgical and Radiologic Anatomy 44(7): 979-981; Riella, J., Tabbara, M. M., Alvarez, A., DeFreitas, M. J., Chandar, J., and 3 others 2022. “Pediatric Kidney Transplants with Multiple Renal Arteries Show No Increased Risk of Complications Compared to Single Renal Artery Grafts,” Online, Frontiers in Pediatrics (Lausanne, Switzerland) 10:1058823; Tabbara, M. M., Guerra, G., Riella, J., Abreu, P., Alvarez, A., and 6 others 2022. “Creating a Single Inflow Orifice from Living Donor Kidney Allografts with Multiple Renal Arteries,” Online, Transplant International 35:10212; Karakaya, E., Akdur, A., Soy, E. A., Moray, G., and Haberal, M. 2021. “Success Rate of Grafts with Multiple Renal Vessels in 3136 Kidney Transplants,” Experimental and Clinical Transplantation 19(1):14-19; OKelly, F., Lorenzo, A. J., Zubi, F., De Cotiis, K., Farhat, W. A., and Koyle, M. A. 2021. “The Impact of Multiple Donor Renal Arteries on Perioperative Complications and Allograft Survival in Paediatric Renal Transplantation,” Journal of Pediatric Urology (Kidlington, Oxford, England) 17(4):541.e1-541. e11; Leighton, P., Hoff, M., Nicholson M. L., and Russell, N. K. 2020. “Dealing with Multiple Renal Arteries in Live Donor Kidney Transplants,” Online, Annals of the Royal College of Surgeons of England 102(9):749-750; Tomizawa, M., Hori. S., Nishimura, N., Omori, C., Nakai, Y., and 3 others 2020. “Arterial Reconstruction Using the Donor's Gonadal Vein in Living Renal Transplantation with Multiple Renal Aarteries: A Case Report and a Literature Review,” BMC [BioMed Central] Nephrology (London, England) 21(1): 190; Al-katib S. Shetty, M., Jafri, S. M., and Jafri, S. Z. 2017. Radiologic Assessment of Native Renal Vasculature: A Multimodality Review,” Online, RadioGraphies 37(1):136-156; O'Kelly, F., Lorenzo, A. J., Zubi, F. De Cotiis, K., Farhat. W. A., and Koyle, M. A. 2011. “The Impact of Multiple Donor Renal Arteries on Perioperative Complications and Allograft Survival in Paediatric Renal Transplantation,” Journal of Pediatric Urology 17(4):541.e1-541.e11; Dogan, S. M., Dogan, G., Simsek, C., Okut, G., Berktas, B., and 7 others 2020. “Transplantation Using Renal Grafts with Multiple Renal Arteries: A Putative Study on the Impact of Arterial Reconstruction Technique and Site of Implantation on Outcomes,” Transplantation Proceedings S0041-1345(20) 30570-30574; Panwar, P., Bansal, D., Maheshwari. R., Chaturvedi, S., Desai, P., and Kumar, A. 2020. “Management of Donor Kidneys with Double Renal Arteries with Significant Luminal Discrepancy: A Retrospective Cohort Study,” Indian Journal of Urology 36(3):200-204; Afriansyah, A., Rasyid, N., Rodjai, A., Wahyudi, I., Mochtar, C. A., Susalit, E., and Hamid, A. R. A. H. 2019. “Laparoscopic Procurement of Single versus Multiple Artery Kidney Allografts: Meta-analysis of Comparative Studies,” Online, Asian Journal of Surgery 42(1):61-70; Gulas. E., Wysiadecki, G., Szymanski, J., Major, A., Stefanczyk, L., Topol, M. and Polguj, M. 2018. “Morphological and Clinical Aspects of the Occurrence of Accessory (Multiple) Renal Arteries,” Online, Archives of Medical Science 14(2):442-453; Al-Katib, S., Shetty, M., Jafri, S. Y., and Jafri, S. 2017. “Radiologic Assessment of Native Renal Vasculature: A Multimodality Review,” RadioGraphies 37(1):136-156; Ashraf, H. S., Hussain, I., Siddiqui, A. A., Ibrahim, M. N., and Khan, M. U. 2013. “The Outcome of Living Related Kidney Transplantation with Multiple Renal Arteries,” Saudi Journal of Kidney Diseases and Transplantation (Riyadh, Saudi Arabia) 24(3):615-619; Ali Mohammed, A. M., Elseed Abdalrasol, R. G., Alamin Abdalhai, K., and Gommaa Hamad, M., 2012. “Accessory Renal Vessels,” Online, Acta Informatica Medica 20(3):196-197; Chabchoub, K., Mhiri, M. N., Bahtoul, A., Fakhfakh, S., Ben Hmida, I., and 5 others 2011. “Does Kidney Transplantation with Multiple Arteries Affect Graft Survival?,” Transplanation Proceedings 43(9):3423-3425; Kapoor, A., Lambe S., Kling, A. L., Piercey, K. R., and Whelan, P. J. 2011. “Outcomes of Laparoscopic Donor Nephrectomy in the Presence of Multiple Renal Arteries,” Online, Urology Annals 3(2):62-65; Hwang, J. K. Kim, S. D., Park, S. C., Choi, B. S., Kim, J. I., and 3 others 2010. “The Long-term Outcomes of Transplantation of Kidneys with Multiple Renal Arteries,” Transplantation Proceedings 42(10):4053-4057; Paramesh, A., Zhang. R., Florman, S., Yau, C. L., McGee, J., and 4 others 2009. “Laparoscopic Procurement of Single versus Multiple Artery Kidney Allografts: Is Long-term Graft Survival Affected?,” Online, Transplantation (Hagerstown, Maryland) 88(10):1203-1207; Mir, N. S., ul Hassan, A., Rangrez, R., Hamid, S., Sabia, Tabish, S. A. Iqbal, and 3 others 2008. “Bilateral Duplication of Renal Vessels: Anatomical, Medical, and Surgical Perspective,” International Journal of Health Sciences (Qassim University, (Riyadh, Saudi Arabia) 2(2):179-185; Başaran, O., Moray, G., Emiroğlu, R., Alevli, F., and Haberal, M. 2004. “Graft and Patient Outcomes among Recipients of Renal Grafts with Multiple Arteries,” Transplantation Proceedings 36(1):102-104; Gurkan, A., Kacar, S. Basak, K., Varilsuha, C., and Karaca, C. 2004. “Do Multiple Renal Arteries Restrict Laparoscopic Donor Nephrectomy?,” Transplantation Proceedings 36(1):105-107; Benedetti, E., Troppmann, C., Gillingham, K., Sutherland. D. E., Payne, W. D., and 4 others 1995. “Short- and Long-term Outcomes of Kidney Transplants with Multiple Renal Arteries,” Online, Annals of Surgery (Philadelphia, Pennsylvania) 221(4):406-414).

Despite the presence of accessory vessels, kidney transplantation in a neonate, infant, or adult with donor and recipient kidneys normal or with accessory blood supply is expedited through the use of a confluence conduit such as that shown in FIG. 35A, the use thereof described below in section 2b(1)(b)(b14), entitled Confluence Conduits: Connection of One or More Bloodlines to Flow through a Common Channel to the Same or a Different Number of Bloodlines with Self-leveling Apportionment of Outflow, shown in nonexpandable form in FIG. 35A.

For use in an infant this can be an expandable conduit as addressed in section 2b(2)(f), entitled Expandable Structure to Accommodate Growth and Self-adjust in Size in Vascular Segment Prostheses, Lines, and Confluence Conduits Placed in Children, as shown in FIG. 36F of a prosthetic aortic neoarch. If the expandable conduit must be too large for eventual implantation when sought to span the distance separating the donor and recipient, a switchable confluence conduit as addressed below in section Long when intercorporeal, to short when intracorporeal with one or multiple confluence conduits, each affording connection of one or more bloodlines to the same or a different number of bloodlines with self-leveling flow apportionment is used wherein the upper of the two lines can be a length or ordinary tubing and the lower an expandable confluence conduit.

Side-to-side ‘pantaloon’ anastomosis of a double donor renal artery to make a single lumen for implantation is conventionally preferred but not always feasible and demands a longer period of hypoxia and patient rewarming (Kim, Y., Kim, M. H., Hwang, J. K., Park, S. C., Kim, J. I., and Jun, K. W. 2021. “Endovascular Treatment for Transplant Renal Artery Stenosis,” Online, Medicine (Baltimore, Maryland) 100(32):e226935; Nataraj, S. A., Zafar, F. A., Ghosh, P. and Ahlawat, R. 2020. “Feasibility and Functional Outcome of Robotic-assisted Kidney Transplantation Using Grafts with Multiple Vessels: Comparison to Propensity Matched Contemporary Open Kidney Transplants Cohort,” Online, Frontiers in Surgery 7:51; Panwar, P., Bansal. D., Maheshwari, R., Chaturvedi, S., Desai, P., and Kumar, A. 2020, Op cit., second preceding paragraph).

Compound vascular bypass is applicable to virtually any anomaly of the kidneys to eliminate ischemia time as could affect graft durability, to include horseshoe, crossed ectopic, and pelvic kidneys, as well as anomalies in other solid organs. Commonly presenting anomalous vasculature, kidneys in particular are likely to be remediated by removal and bench vascular reconstruction (see, for example. Tabbara. M. M., Guerra, G., Riella, J., Abreu, P., Alvarez, A., and 6 others 2022. Op cit. in this section; Hevia, V., Gomez, V., Hevia, M., Lorca, J., Santiago, M. and 5 others 2020. “Troubleshooting Complex Vascular Cases in the Kidney Transplant Recipient: Vascular Anomalies, Challenging Vessel Diseases and Procedural Disasters,” Current Urology Reports 21(1):7; Hevia. V., Gomez, V., Hevia, M., Lorca, J., Santiago, M. and 5 others 2020. “Troubleshooting Complex Vascular Cases in the Kidney Graft: Multiple Vessels, Aneurysms, and Injuries during Harvesting Procedures,” Current Urology Reports 21(1):5; Rossetto, A., Comai, G., Cuna. V., Siniscalchi, A., Corradetti. V., and 10 others 2020. “Double Single-side Kidney Transplants with Bench Vascular Reconstruction: A Further Challenge beyond the Marginality without Future Preclusions,” Transplantation Proceedings 52(5):1544-1546; Watson, C. J. E. and Harper, S. J. F. 2015. “Anatomical Variation and Its Management in Transplantation,” Online, American Journal of Transplantation 15(6):1459-1471).

In that pantaloon anastomosis combines the flow of two vessels, it shares a measure of functional similarity with a confluence conduit, which however, can combine the flow of more than two input vessels into a common channel which also unlike the organic anastomosis, can selectively medicate or increase or reduce the blood pressure by means of an inline through-flow pump in the conduit. The use of a switchable confluence conduit allows the operation to proceed without the relative complexity and potential degradation to the graft organ due to storage by any means and whether of a right or left donor kidney with or without accessory arteries to replace a native right or left recipient kidney with or without accessory arteries. Whether heterotopic positioning would add to durability and/or reduce the incidence of tubercular lung reinfection, for example, warrants study.

2b(1)(b)(b9). Single Living and Single or Double Deceased Donor Metered Compound Vascular Bypass, or Switched-Organ, Lung Transplantation

Where a proper donor sustainment program with regional hub deceased maintenance specialty centers and shuttle air support to deliver patients would overcome the shortcoming, a graft organ usually becomes available only after the donor has died. If in the hospital, terminal, and available for post-expiration sustainment, rather than prepared thus, the donor is allowed to die first and the cadaveric organs preserved with an artificial chilled static or flowing perfusate. This gross mishandling at the outset allows the organ to have undergone considerable trauma associated not only with the termination of oxygenation and perfusion but the chemical upheaval associated with dying and death.

Genetics accepted, for the variables which might be controlled, continuity is everything: for any transplant, the prospect of a successful outcome is increased when the graft organ has never experienced an interruption, much less extended, in normal function. So due to mismanagement, these factors are allowed to join with immune intolerance to severely reduce the odds for a successful result. While proper management would have instituted closer to uninterrupted continuity than is currently possible without the diversion valving required, a key factor responsible for this situation is that uninterrupted continuity of circulatory and respiratory function assumes the availability of intravascular diversion solenoid and servovalves.

In compound vascular bypass, or switch-organ transplantation, the object is to remove the graft organ from the respiratory and circulatory systems of the donor and transfer it into the respiratory and circulatory systems of the recipient with no interruption in the continuity of graft perfusion. Sudden compound vascular bypass, or sudden switched-organ transplantation responds to urgency not ordinarily encountered in lung transplantation so that lung transplantation is usually undertaken using the metered switch or gradual transfer technique, and in so doing, making it possible for the graft organ and recipient to gain a measure of immune tolerance induction at the outset.

When the prospective graft lung is not clearly acceptable for transplant, treatment can either precede transplantation by means of ex vivo lung perfusion, or if the sensors to detect adverse eventualities are available, the transplantation can be performed using the metered switch technique with follow-up therapy accomplished by the implant automatic response system. Accordingly, the operation can be preceded by testing a portion of the prospective graft by ex vivo lung perfusion. Whether reconditioning is ex vivo, is to follow transplantation, or a combination thereof will depend upon whether the lung can be implanted first, and if not, then the degree of therapy required to bring the lung up to an implantable standard. Ex vivo lung perfusion as a remote preservation means when the lung is plainly suitable for transplantation is gratuitous and inferior to immediate switch transplantation. It should be applied only when necessary.

The proper uses of this technique, justified on the basis of a clear need, are to 1. Preserve the graft organ when transportation to or by the prospective recipient is not feasible, 2. Pretest to disqualify a marginal organ when therapy does not impart sufficient improvement, and 3. Eliminate the need to guess whether the organ would recover once implanted. For the latter purpose, an additional consideration to qualify an organ that would ordinarily be rejected is that an implanted automatic response system, because it directly targets and properly times the release of medication to the graft, can do so in increased doses while avoiding the rest of the body and the adverse side effects of conventional systemic drugging.

As a preliminary measure applied to a small portion of a clearly marginal prospective graft lung to allow diagnosis and treatment prior to transplantation and optimization of the post-transplantation prescription-program, ex vivo lung perfusion is of demonstrable value (see, for example, Ghaidan, H., Fakhro, M., Andreasson, J., Pierre, L., Ingemansson, R., and Lindstedt, S. 2019. “Ten Year Follow-up of Lung Transplantations Using Initially Rejected Donor Lungs after Reconditioning Using ex Vivo Lung Perfusion,” Online, BioMed Central Journal of Cardiothoracic Surgery (London, England) 14(1):125).

Ex vivo lung perfusion is also of value for confirming the inadequacy and averting the futile transplantation of a lung which treatment in advance establishes would fail to respond to therapy were it implanted (see, for example, Possoz, J., Neyrinck, A., and Van Raemdonck, D. 2019. “Ex Vivo Lung Perfusion Prior to Transplantation: An Overview of Current Clinical Practice Worldwide,” Journal of Thoracic Disease 11(4):1635-1650; Abdalla, L. G., Braga, K. A., Nepomuceno, N. A., Fernandes, L. M., Samano, M. N. Pego-Fernandes P. M. 2016. “Ex Vivo Lung Perfusion in Brazil,” Online, Jornal Brasileiro de Pneumologia [Brazilian Journal of Pneumonology] (Brasilia, Brazil) 42(2):95-98). That is, when confronted with a questionable graft organ, ex vivo lung perfusion allows greater confidence in accepting or rejecting the lung for transplantation.

Lung transplantation is an option in severe pulmonary arterial Hypertension (see, for example, Hu, X., Ding, N., Songchen, W., Wang, R., Chen, J., and 5 others 2023. “Lung Transplantation for Pulmonary Arterial Hypertension: Optimized Referral and Listing Based on an Evolving Disease Concept,” Online, MDPI [Multidisciplinary Digital Publishing Institute] Journal of Cardiovascular Development and Disease (Basel, Switzerland) 10(8):350; (see also Miller, C. L., O, J. M., Allan, J. S, and Madsen, J. C. 2022. “Novel Approaches for Long-term Lung Transplant Survival,” Online, Frontiers in Immunology (Lausanne, Switzerland) 13:931251; also cited herein in section 2a above, entitled Background of the Invention. General, and section 2b(2)(h)(h3) below, entitled The ‘Tuned’ Shunt in the Relief of Pulmonary Arterial Hypertension).

If applicable, the findings are incorporated into the prescription-program that will administer the transfer of the lung to the recipient from the living or deceased donor sustained on extracorporeal membrane oxygenation and/or direct stimulation of the circulatory and respiratory centers in the brainstem, as addressed above in section 2a. This preliminary removal of a portion of the graft lung is performed endoscopically under local anesthesia using a chest dome as described above in section 2a and below in sections 2b(2)(g), entitled Ischemia and Hypoperfusion-free Carotid Endarterectomy Bypass and Prosthesis and 6, entitled Description of the Preferred Embodiments of the Invention. Once ex vivo lung perfusion has been accomplished, the portion of lung removed is returned to the lung in the same manner it was removed.

In this way, advancements in conventional lung transplantation which allow diagnosis and treatment of a questionable graft can be incorporated into the metered switch method, which 1. Using the information provided through ex vivo lung perfusion, 2. Blunting the immune reaction in a targeted rather than systemic manner from the outset, 3. Allowing uninterrupted perfusion and oxygenation, and 4. Fighting off infection, averts much of the causes for graft failure (see, for example, Abdalla, L. G., de Oliveira-Braga, K. A., Fernandes, L. M., Samano, M. N., Camerini, P. R., and Pego-Fernandes, P. M. 2019. “Evaluation and Reconditioning of Donor Organs for Transplantation through ex Vivo Lung Perfusion,” Einstein (Sao Paulo, Brazil) 17(4):eAO4288; Aslam, S., Courtwright, A. M., Koval, C., Lehman, S. M., Morales, S., and 18 others 2019. “Early Clinical Experience of Bacteriophage Therapy in 3 Lung Transplant Recipients,” American Journal of Transplantation 19(9):2631-2639; Guenthart, B. A., O'Neill, J. D., Kim, J., Queen, D., Chicotka, S., and 12 others 2019. “Regeneration of Severely Damaged Lungs Using an Interventional Cross-circulation Platform,” Nature Communications 10(1):1985; Slama, A., Raber, C., Hedderich, C., Stockhammer, P., Hegedus, B., and 4 others 2019. “Implementation of an Experimental Isolated Lung Perfusion Model on Surgically Resected Human Lobes,” (Nature Research) Scientific Reports 9(1):12193; DerHovanessian, A., Wallace, W. D., Lynch, J. P. 3rd, Belperio, J. A., and Weigt, S. S. 2018. “Chronic Lung Allograft Dysfunction: Evolving Concepts and Therapies,” Seminars in Respiratory and Critical Care Medicine 39(2):155-171; Hsin, M. and Au. T. 2018. “Ex Vivo Lung Perfusion: A Potential Platform for Molecular Diagnosis and ex Vivo Organ Repair,” Journal of Thoracic Disease 10 (Supplement 16):S1871-S1883; Ivulich, S., Westall, G., Dooley, M., and Snell. G. 2018. “The Evolution of Lung Transplant Immunosuppression,” Drugs (Auckland, New Zealand) 78(10):965-982; Meyer, K. C. 2018. “Recent Advances in Lung Transplantation,” Faculty of 1000 Research 7. pii: F1000 Faculty Rev-1684; Nakajima, D., Liu, M., Ohsumi, A., Kalaf. R., Iskender, I., and 15 others 2017. “Lung Lavage and Surfactant Replacement during ex Vivo Lung Perfusion for Treatment of Gastric Acid Aspiration-induced Donor Lung Injury,” Journal of Heart and Lung Transplantation (New York, New York) 36(5):577-585; Pan, X., Yang, J., Fu. S., and Zhao, H. 2018. “Application of Ex Vivo Lung Perfusion (EVLP) in Lung Transplantation,” Journal of Thoracic Disease 10(7):4637-4642; Yeung, J. C., Cypel. M., and Keshavjee, S. 2017. “Ex-vivo Lung Perfusion: The Model for the Organ Reconditioning Hub,” Current Opinion in Organ Transplantation 22(3):287-289; Cypel, M. and Keshavjee, S. 2016. “Extracorporeal Lung Perfusion (ex-Vivo Lung Perfusion),” Current Opinion in Organ Transplantation 21(3):329-335; Reeb, J. and Cypel, M. 2016. “Ex Vivo Lung Perfusion,” Clinical Transplantation 30(3):183-194 also cited under section 2a(3), entitled Remotely Procured Graft Organ Preservation; Cypel, M. and Keshavjee, S. 2015. “Extending the Donor Pool: Rehabilitation of Poor Organs,” Thoracic Surgery Clinics 25(1):27-33; Cervera, C., van Delden, C., Gavalda, J., Welte. T., Akova, M., and Carratala, J. 2014. “Multidrug-resistant Bacteria in Solid Organ Transplant Recipients,” Clinical Microbiology and Infection 20 Supplement 7:49-73; Machuca. T. N. and Cypel, M. 2014. “Ex Vivo Lung Perfusion,” Journal of Thoracic Disease 6(8):1054-1062; Cypel, M. and Keshavjee, S. 2011. “Extracorporeal Lung Perfusion,” Current Opinion in Organ Transplantation 16(5):469-475).

Moreover, the automatic system which administers the operation thereafter remains implanted in the recipient where it detects and responds to any sign of degradation (see, for example, Costa, J., Benvenuto, L. J., and Sonett, J. R. 2017. “Long-term Outcomes and Management of Lung Transplant Recipients,” Best Practice and Research. Clinical Anesthesiology 31(2):285-297; Potestio. C., Jordan, D., and Kachulis, B. 2017. “Acute Postoperative Management after Lung Transplantation,” Best Practice and Research. Clinical Anesthesiology 31(2): 273-284; Shino, M. Y., Weigt. S. S., Li, N., Derhovanessian, A., Sayah, D. M., and 8 others 2017. “Impact of Allograft Injury Time of Onset on the Development of Chronic Lung Allograft Dysfunction after Lung Transplantation,” American Journal of Transplantation 17(5):1294-1303; Verleden, S. E., Gottlieb, J., Dubbeldam, A., Verleden, G. M., Suhling, H., and 3 others 2017. “White-Out” after Lung Transplantation: A Multicenter Cohort Description of Late Acute Graft Failure,” American Journal of Transplantation 17(7):1905-1911; Robson, K. S. and West, A. J. 2014. “Improving Survival Outcomes in Lung Transplant Recipients through Early Detection of Bronchiolitis Obliterans: Daily Home Spirometry versus Standard Pulmonary Function Testing,” Canadian Journal of Respiratory Therapy 50(1):17-22).

For example, allogeneic hematopoietic stem cell transplantation is usually administered to treat a hematological or malignant disorder such as a leukemia, lymphoma, myeloma, polycythemia vera, thalassemia, or sickle cell disease, rather than to support a lung or other solid organ transplant. Bronchiolitis obliterans then appears due to rejection of and by the new lung or lungs, or because the new lung or lungs are less able to withstand the aspiration of refluxed gastric contents.

The development of bronchiolitis obliterans following lung transplantation can arise for reasons other than lung or hematopoietic stem cell transplant rejection-“Bile acid aspiration is associated with impaired lung allograft innate immunity manifest by reduced surfactant collectins [innate immune system collagen-containing C-type lectins] and altered phospholipids” (D'Ovidio, F., Mura, M., Ridsdale, R., Takahashi, H., Waddell, T. K., and 11 others 2006. “The Effect of Reflux and Bile Acid Aspiration on the Lung Allograft and Its Surfactant and Innate Immunity Molecules SP-A and SP-D,” American Journal of Transplantation 6(8):1930-1938):(see also Guenthart. B. A., O'Neill, J. D., Kim, J., Queen, D., Chicotka, S., and 12 others 2019. Op cit., this section; Biswas. M., Kumar, S. R. P., Terhorst. C., and Herzog, R. W. 2018. “Gene Therapy with Regulatory T Cells: A Beneficial Alliance,” Online, Frontiers in Immunology (Lausanne, Switzerland) 9:554; Schott, C., Weigt. S. S., Turturice, B. A., Metwally, A., Belperio, J., Finn, P. W., and Perkins, D. L. 2018. “Bronchiolitis Obliterans Syndrome Susceptibility and the Pulmonary Microbiome,” Journal of Heart and Lung Transplantation (New York, New York) 37(9):1131-1140; Hathom, K. E., Chan, W. W., and Lo. W. K. 2017. “Role of Gastroesophageal Reflux Disease in Lung Transplantation,” Online, World Journal of Transplantation (Pleasanton, California) 7(2):103-116; Nakajima, D., Liu, M., Ohsumi, A., Kalaf. R., Iskender, I., and 15 others 2017, Op cit. this section; Todd, J. L. and Palmer, S. M. 2011. “Bronchiolitis Obliterans Syndrome: The Final Frontier for Lung Transplantation,” Chest 140(2):502-508; D'Ovidio, F., Mura, M., Tsang, M., Waddell, T. K., Hutcheon, M. A. and 8 others 2005. “Bile Acid Aspiration and the Development of Bronchiolitis Obliterans after Lung Transplantation,” Journal of Thoracic and Cardiovascular Surgery 129(5):1144-1152).

The addition of a reflux sensor and nonjacketing side-entry connector near to the lower esophageal sphincter can be used by the same system to directly pipeline target sodium bicarbonate a proton pump inhibitor, or histamine H2 receptor antagonist, for example, prepared in fluid form to the lower esophageal sphincter and gastric cardia as well as immunosuppressants to the blood supply of the lung transplant. The use of a microprocessor in lieu of a simpler microcontroller allows the system to respond to comorbid conditions, here the risk of rejection in the respiratory system and acid reflux in the gastrointestinal tract. The system can therefore reduce the incidence of induced bronchiolitis obliterans or reduce its severity, whether due to impaired immunity of the transplant or host versus graft rejection. Hence, the automatic neutralization of acid that would otherwise reflux to be aspirated and the automatic release of immunosuppressants should significantly retard if not preclude the development of bronchiolitis obliterans.

If the spirometry performed as a routine check for the development of bronchiolitis obliterans following lung transplantation cannot be accomplished with adequate frequency, the system can be made to signal and respond directly to the appearance of bronchiolitis obliterans (see, for example, Hakim, A., Cooke, K. R., Pavletic, S. Z., Khalid, M., Williams, K. M., and Hashmi, S. K. 2019. “Diagnosis and Treatment of Bronchiolitis Obliterans Syndrome Accessible Universally,” Bone Marrow Transplantation 54(3):383-392; Ruttens. D., Verleden, S. E., Demeyer, H., Van Raemdonck, D. E., Yserbyt. J., and 4 others 2018. “Montelukast for Bronchiolitis Obliterans Syndrome after Lung Transplantation: A Randomized Controlled Trial,” Online, PLoS [Public Library of Science] One 13(4):e0193564; Benden, C., Haughton, M., Leonard, S., and Huber, L. C. 2017. “Therapy Options for Chronic Lung Allograft Dysfunction-Bronchiolitis Obliterans Syndrome following First-line Immunosuppressant Strategies: A Systematic Review,” Journal of Heart and Lung Transplantation (New York, New York) 36(9): 921-933; Koutsokera, A., Royer, P. J., Antonietti, J. P., Fritz. A., Benden, C., and 20 others 2017. “Development of a Multivariate Prediction Model for Early-onset Bronchiolitis Obliterans Syndrome and Restrictive Allograft Syndrome in Lung Transplantation,” Online, Frontiers in Medicine (Lausanne, Switzerland) 4:109; Kim, S. W., Rhee, C. K., Kim, Y. J., Lee, S., Kim, H. J., and Lee, J. W. 2016, Op cit.; Borthwick, L. A. 2016. “The IL-1 Cytokine Family and Its Role in Inflammation and Fibrosis in the Lung,” Seminars in Immunopathology 38(4):517-534; Belperio, J. A., DiGiovine, B., Keane, M. P., Burdick, M. D., Ying Xue, Y., and 4 others 2002. “Interleukin-1 Receptor Antagonist as a Biomarker for Bronchiolitis Obliterans Syndrome in Lung Transplant Recipients,” Transplantation 73(4):591-599; Nord. M., Schubert, K., Cassel. T. N., Andersson, O., and Riise, G. C. 2002. “Decreased Serum and Bronchoalveolar Lavage Levels of Clara Cell Secretory Protein (CC16) is Associated with Bronchiolitis Obliterans Syndrome and Airway Neutrophilia in Lung Transplant Recipients,” Transplantation 73(8):1264-1269) and automatically suppress its progression through the directly lung-targeted release of azithromycin, montelukast, and fluticasone propionate prepared in fluid form. Hematopoietic stem cells are a ‘two-edged sword’ which can promote healing or provoke an immune reaction and is associated with viral infection that results in death (Sheshadri, A., Chemaly, R. F., Alousi, A. M., Shah, P. K., Rondon, G., and 8 others 2019. Op cit.).

Methods for the prevention of bronchiolitis obliterans are under study (see, for example Smirnova, N. F., Conlon, T. M., Morrone, C., Dorfmuller, P., Humbert. M., and 5 others 2019. “Inhibition of B Cell-dependent Lymphoid Follicle Formation Prevents Lymphocytic Bronchiolitis after Lung Transplantation,” Journal of Clinical Investigation Insight 4(3). pii: 123971; Meyer, K. C. 2018. “Recent Advances in Lung Transplantation,” Faculty of 1000 Research 7. pii: F1000 Faculty Rev-1684; Lynch, T. J., Ahlers. B. A., and Parekh, K. R. 2018. “Lung Transplantation: Chronic Rejection and Stem Cell Depletion,” Journal of Thoracic Disease 10(8):E666-E668; Pierog, J., Tamo, L., Fakin, R., Kocher, G., Gugger, M., and 4 others 2018. “Bone Marrow Stem Cells Modified with Human Interleukin 10 Attenuate Acute Rejection in Rat Lung Allotransplantation,” European Journal of Cardiothoracic Surgery 53(1):194-200; Verghese, D. A., Chun, N., Paz, K., Fribourg, M., Woodruff, T. M., and 8 others 2018. “C5aR1 [complement component C5a anaphylatoxin chemotactic receptor 1/cluster of differentiation 88] Regulates T Follicular Helper Differentiation and Chronic Graft-versus-host Disease Bronchiolitis Obliterans,” Journal of Clinical Investigation Insight 3(24). pii: 124646; Watanabe, S., Kasahara, K., Waseda, Y., Takato, H., Nishikawa, S., and 6 others 2017. “Imatinib Ameliorates Bronchiolitis Obliterans via Inhibition of Fibrocyte Migration and Differentiation,” Journal of Heart and Lung Transplantation (New York, New York) 36(2):138-147; Sato, S., Shinohara, S., Hayashi, S., Morizumi, S., Abe, S., and 11 others 2017. “Anti-fibrotic Efficacy of Nintedanib in Pulmonary Fibrosis via the Inhibition of Fibrocyte Activity,” Respiratory Research 18(1):172; Walton, D. C., Hiho, S., Cantwell, L. S., Diviney. M. B., Wright, S. T., and 3 others 2016. “HLA Matching at the Eplet Level Protects against Chronic Lung Allograft Dysfunction,” American Journal of Transplantation 16(9):2695-2703; Watanabe, S., Waseda, Y., Kimura, H., Takato, H., Ohata. K., and 3 others 2015. “Imatinib for Bronchiolitis Obliterans after Allogeneic Hematopoietic Stem Cell Transplantation,” Bone Marrow Transplantation 50(9):1250-1252; Ye. F., Lecourt, S., Vernochet, A., and Durrbach, A. 2015. “Utilisation des Cellules Souches Mesenchymateuses en transplantation d'Organe,” [Use of Mesenchymal Stem Cells in Organ Transplantation], (in French with abstract at Pubmed), Bulletin de l'Académie Nationale de Médecine [Bulletin of the National Academy of Medicine] 199(4-5):515-523; Dubovsky, J. A., Flynn, R., Du, J., Harrington, B. K., Zhong. Y., and 25 others 2014. “Ibrutinib Treatment Ameliorates Murine Chronic Graft-versus-host Disease,” Journal of Clinical Investigation 124(11):4867-4876; Kim, Y. R., Haam, S., Park, Y. G., Lim, B. J., Park, Y. M., and Paik, H. C. 2012. “Lung Transplantation for Bronchiolitis Obliterans after Allogeneic Hematopoietic Stem Cell Transplantation,” Yonsei Medical Journal 53(5):1054-1057; Forslow, U., Mattsson, J., Gustafsson, T., and Remberger, M. 2011. “Donor Lymphocyte Infusion May Reduce the Incidence of Bronchiolitis Obliterans after Allogeneic Stem Cell Transplantation” Biology of Blood and Marrow Transplantation 17(8):1214-1221; Chien, J. W. 2011, Op cit.), and the implanted automatic disorder response system can not only release stem cells, preventive agents, immunosuppressants, and reversal agents but telemetrically alert the clinic to the condition.

The condition can also be triggered by graft/host mutual rejection following lung transplantation, stem cell therapy, and/or antimicrobial administration (see, for example, Chambers, D. C. 2019. “Bronchiolitis Obliterans Syndrome ‘Endotypes’ in Haematopoietic Stem Cell Transplantation,” Respirology 24(5):408-409; Yomota, M., Yanagawa, N., Sakai, F., Yamada, Y., Sekiya, N., Ohashi, K., and Okamura, T. 2019. “Association between Chronic Bacterial Airway Infection and Prognosis of Bronchiolitis Obliterans Syndrome after Hematopoietic Cell Transplantation,” Medicine (Baltimore, Maryland) 98(1):e13951; Sheshadri, A., Chemaly, R. F., Alousi, A. M., Shah, P. K., Rondon, G., and 8 others 2019. “Pulmonary Impairment after Respiratory Viral Infections is Associated with High Mortality in Allogeneic Hematopoietic Cell Transplant Recipients,” Biology of Blood and Marrow Transplantation 25(4):800-809; Pochon, C. and Voigt. S. 2019. “Respiratory Virus Infections in Hematopoietic Cell Transplant Recipients,” Online, Frontiers in Microbiology 9:3294; Pochon, C. and Voigt S. 2019. “Respiratory Virus Infections in Hematopoietic Cell Transplant Recipients,” Online, Frontiers in Microbiology 9:3294; Chen-Yoshikawa, T. F., Sugimoto, S., Shiraishi, T., Minami, M., Matsuda, Y., and 9 others 2018. “Prognostic Factors in Lung Transplantation after Hematopoietic Stem Cell Transplantation,” Transplantation 102(1):154-161; Ogimi, C., Krantz, E. M., Golob, J. L., Waghmare, A., Liu, C., and 10 others 2018. “Antibiotic Exposure Prior to Respiratory Viral Infection is Associated with Progression to Lower Respiratory Tract Disease in Allogeneic Hematopoietic Cell Transplant Recipients,” Biology of Blood and Marrow Transplantation 24(11):2293-2301; Williams, K. M. 2017. “How I Treat Bronchiolitis Obliterans Syndrome after Hematopoietic Stem Cell Transplantation,” Blood 129(4):448-455; Rhee, C. K., Ha, J. H., Yoon, J. H., Cho, B. S., Min, W. S., Yoon, H. K., and Lee, J. W. 2016. “Risk Factor and Clinical Outcome of Bronchiolitis Obliterans Syndrome after Allogeneic Hematopoietic Stem Cell Transplantation,” Yonsei Medical Journal 57(2):365-372; Jung. H. S., Lee, J. G., Yu, W. S., Lee, C. Y., Haam, S. J., and Paik, H. C. 2016. “Early Outcomes of Lung Transplantation for Bronchiolitis Obliterans Syndrome after Allogeneic Haematopoietic Stem Cell Transplantation: A Single-centre Experience,” Interactive Cardiovascular and Thoracic Surgery 23(6):914-918; Kim, S. W., Rhee, C. K., Kim, Y. J., Lee, S., Kim, H. J., and Lee, J. W. 2016. “Therapeutic Effect of Budesonide/Formoterol, Montelukast and N-acetylcysteine for Bronchiolitis Obliterans Syndrome after Hematopoietic Stem Cell Transplantation,” Respiratory Research 17(1):63; Williams, K. M., Cheng. G. S., Pusic. I., Jagasia, M., Burns. L.5., and 14 others 2016. “Fluticasone, Azithromycin, and Montelukast Treatment for New-onset Bronchiolitis Obliterans Syndrome after Hematopoietic Cell Transplantation,” Biology of Blood and Marrow Transplantation 22(4):710-716; Sengsayadeth, S. M., Srivastava, S., Jagasia, M., and Savani, B. N. 2012. “Time to Explore Preventive and Novel Therapies for Bronchiolitis Obliterans Syndrome after Allogeneic Hematopoietic Stem Cell Transplantation,” Biology of Blood and Marrow Transplantation 18(10):1479-1487; Au, B. K., Au, M. A., and Chien, J. W. 2011. “Bronchiolitis Obliterans Syndrome Epidemiology after Allogeneic Hematopoietic Cell Transplantation,” Biology of Blood and Marrow Transplantation 17(7):1072-1078; Chien, J. W. 2011. “Preventing and Managing Bronchiolitis Obliterans Syndrome after Allogeneic Hematopoietic Cell Transplantation,” Expert Review of Respiratory Medicine 5(1):127-135).

At the same time that bronchiolitis obliterans is indicative of transplant rejection, the impaired immunity of the graft organ or organs due to the administration of immunosuppressants leaves the lungs less able to withstand the injury imposed by gastroesophageal-induced aspiration of bile acid. Numerous means, mechanical, surgical, and pharmaceutical, exist to suppress gastroesophageal reflux. Those mechanical or electromechanical including artificial sphincters to supplement the lower esophageal, such as that shown in FIG. 11 of U.S. Pat. No. 11,759,186 and the passive LINX® Reflux Management System (Torax® Medical, Incorporated, Saint Paul, Minnesota), a segmented compression magnetized bead-based ring type sphincter.

Bronchiolitis obliterans and certain other conditions of the lung such as chronic obstructive pulmonary disease and pulmonary fibrosis are irreversible, and lungs affected thus should rejected for transplantation, even by the metered switch technique. By the same token, lung transplantation, especially bilateral, makes survival possible for patients affected thus (see, for example, Fakhro, M., Broberg. E., Algotsson, L., Hansson, L., Koul. B., and 4 others 2017. “Double Lung, Unlike Single Lung Transplantation Might Provide a Protective Effect on Mortality and Bronchiolitis Obliterans Syndrome,” Journal of Cardiothoracic Surgery 12(1):100).

Lungs determined to be marginal due to curable disease may be considered for transplantation. In lieu of biopsy histological examination, mediators of the immune response, such as T cells, B cells, and the biomarkers specified above are detected with the aid of small implanted sensors available or in development. Section 2b(1)(b), entitled Metered Compound Vascular Bypass, or Metered Switched-organ Transplantation, addresses sensors more generally than those intended to detect signs or biomarkers of organ rejection.

Detected, the system can respond by directly pipeline targeting medication to the graft organ and/or the site or sites, typically, the liver and/or the gut, of mediator generation. The direct targeting of an immunosuppressant and antimicrobial to the lung itself, liver, and gut, for example, can not only be administered in higher than systemic dose but avoids the risk serious side effects such as increased susceptibility to infection, thus allowing more aggressive suppression of spirometry and experiential signs of impending rejection.

At the same time, a background systemic dose, also deliverable by the implanted system, is released to treat any root root, or site, of disease of rejection that may have gone undetected (see, for example, Kaundal. U., Bagai, U., and Rakha, A. 2018, Op cit. above in sections 2a, entitled Background of the Invention, General, and 2b(1)(b)(b2), entitled Prenatal Compound Vascular Bypass. or Sudden Switched-organ Transplantation of the Heart; Gao, F., Chiu, S. M., Motan, D. A., Zhang, Z., Chen, L., and 4 others 2016. “Mesenchymal Stem Cells and Immunomodulation: Current Status and Future Prospects,” Cell Death and Disease 7: e2062). The transplantation of lungs marginal due to reversible disease is best bilateral, limiting living donors to one lung each, and necessitating a full clamshell thoracotomy under a chest dome as described above in section 2a, entitled Background of the Invention. General, and below in sections 2b(2)(g), entitled Ischemia and Hypoperfusion-free Carotid Endarterectomy Bypass and Prosthesis, and 6, entitled Description of the Preferred Embodiments of the Invention.

While more complex, it has been established that double lung transplantation is usually preferable, as it replaces any diseased or disease-susceptible tissue in the contralateral lung capable of a recrudescence (see, for example, Santambrogio, L., Tarsia, P., Mendogni, P., and Tosi, D. 2018. “Transplant Options for End Stage Chronic Obstructive Pulmonary Disease in the Context of Multidisciplinary Treatments,” Journal of Thoracic Disease 10 (Supplement 27):S3356-S3365; Subramanian, M. P. and Meyers, B. F. 2018. “Bilateral versus Single Lung Transplantation: Are Two Lungs Better than One?,” Journal of Thoracic Disease 10(7):4588-4601; Fakhro, M., Broberg, E., Algotsson, L., Hansson, L., Koul, B., and 4 others 2017, Op cit., this section; Hartwig, M. G., Ganapathi, A. M., Osho, A. A., Hirji, S. A., Englum, B. R., and 4 others 2016. “Staging of Bilateral Lung Transplantation for High-risk Patients with Interstitial Lung Disease: One Lung at a Time,” American Journal of Transplantation 16(11):3270-3277; Puri, V., Patterson, G. A., and Meyers, B. F. 2015. “Single versus Bilateral Lung Transplantation: Do Guidelines Exist?,” Thoracic Surgery Clinics 25(1):47-54; Schaffer, J. M., Singh, S. K., Reitz, B. A., Zamanian, R. T., and Mallidi, H. R. 2015. “Single-vs Double-lung Transplantation in Patients with Chronic Obstructive Pulmonary Disease and Idiopathic Pulmonary Fibrosis Since the Implementation of Lung Allocation Based on Medical Need,” Journal of the American Medical Association 313(9):936-948; Hayanga, J. W. and D'Cunha, J. 2014. “The Surgical Technique of Bilateral Sequential Lung Transplantation,” Journal of Thoracic Disease 6(8):1063-1069; Yeung, J. C. and Keshavjee S. 2014. “Overview of Clinical Lung Transplantation,” Cold Spring Harbor Perspectives in Medicine 4(1):a015628; Force, S. D., Kilgo, P., Neujahr, D. C., Pelaez, A., Pickens, A., and 3 others 2011. “Bilateral Lung Transplantation Offers Better Long-term Survival, Compared with Single-lung Transplantation, for Younger Patients with Idiopathic Pulmonary Fibrosis,” Annals of Thoracic Surgery 91(1):244-249; Wang, Q., Rogers, C. A., Bonser, R. S., Banner, N. R., Demiris, N., and Sharples, L. D. with 13 collaborators 2011. “Assessing the Benefit of Accepting a Single Lung Offer Now Compared with Waiting for a Subsequent Double Lung Offer,” Transplantation 91(8):921-926; Neurohr, C., Huppmann, P., Thum, D., Leuschner, W., von Wulffen, W., and 10 others 2010. “Potential Functional and Survival Benefit of Double over Single Lung Transplantation for Selected Patients with Idiopathic Pulmonary Fibrosis,” Transplant International 23(9):887-896; Weiss, E. S., Allen, J. G., Merlo, C. A., Conte, J. V., and Shah, A. S. 2009. “Survival after Single versus Bilateral Lung Transplantation for High-risk Patients with Pulmonary Fibrosis,” Annals of Thoracic Surgery 88(5):1616-1626; Thabut, G., Christie, J. D., Ravaud, P., Castier, Y., Brugiere, O., and 4 others 2008. “Survival after Bilateral versus Single Lung Transplantation for Patients with Chronic Obstructive Pulmonary Disease: A Retrospective Analysis of Registry Data,” Lancet (London, England) 371(9614):744-751; Nwakanma, L. U., Simpkins, C. E., Williams, J. A., Chang, D. C., Borja, M. C., Conte, J. V., and Shah, A. S. 2007. “Impact of Bilateral versus Single Lung Transplantation on Survival in Recipients 60 Years of Age and Older: Analysis of United Network for Organ Sharing Database,” Journal of Thoracic and Cardiovascular Surgery 133(2):541-547; Hadjiliadis, D. and Angel, L. F. 2006. “Controversies in Lung Transplantation: Are Two Lungs Better than One?,” Seminars in Respiratory and Critical Care Medicine 27(5):561-566).

While metered compound vascular bypass, or sudden switched-organ transplantation should significantly reduce post-transplantation drug-responsive immune complications, except in a metered double heart transplant, an adverse size mismatch must still be dealt with by conventional means. The literature inconsistent in this regard, significant oversize of an intact graft compared to the native lung usually results in additional complications which can be avoided through lobar transplantation, while significant undersize can result in the need for tracheostomy and extended post-transplantation ventilation.

Rather than size, in bilateral and likely unilateral lung transplantation, increased total lung capacity and not just larger lung size is likely to yield a more favorable outcome (see, for example, Xu, Y., Qing, Q., Liang, M., Liang, W., Lin, Z., and 6 others 2018. “Diaphragm Electromyography Guidance for a Lung Transplant Recipient with Difficult Weaning from Mechanical Ventilation: A Case Report,” Medicine (Baltimore, Maryland) 97(23):e10989; Hwang, S. H., Lee, J. G., Kim, T. H., Paik, H. C., Park, C. H., and Haam, S. 2016. “Comparison of Predicted Total Lung Capacity and Total Lung Capacity by Computed Tomography in Lung Transplantation Candidates,” Yonsei Medical Journal (Seoul, South Korea) 57(4):963-967; Eberlein, M., Amaoutakis, G. J., Yarmus, L., Feller-Kopman, D., Dezube, R., and 9 others 2012. “The Effect of Lung Size Mismatch on Complications and Resource Utilization after Bilateral Lung Transplantation,” Journal of Heart Lung Transplantation 31(5):492-500).

The risk of primary graft dysfunction in bilateral transplantation has been reported to be reduced in recipients transplanted for a cause other than chronic occlusive pulmonary disease, or pulmonary arterial hypertension (Eberlein, M., Reed, R. M., Bolukbas, S., Diamond, J. M., Wille, K. M., and 3 others 2015. “Lung Size Mismatch and Primary Graft Dysfunction after Bilateral Lung Transplantation,” Journal of Heart and Lung Transplantation (New York, New York) 34(2):233-240; Eberlein, M., Diehl, E., Bolukbas, S., Merlo, C. A., and Reed, R. M. 2013. “An Oversized Allograft is Associated with Improved Survival after Lung Transplantation for Idiopathic Pulmonary Arterial Hypertension,” Journal of Heart and Lung Transplantation (New York, New York) 32(12):1172-1178).

The question as to whether transplanting a lung or lobe into the contralateral position is to be avoided has been answered in the negative (see, for example, Goda, Y., Motoyama, H., Aoyama, A., Hamaji, M., Hijiya, K., Chen-Yoshikawa, T. F., and Date, H. 2017. “Right-to-Left Inverted Living-donor Lobar Lung Transplantation Combined with Sparing of Native Right Upper Lobe,” Journal of Heart and Lung Transplantation (New York, New York) 36(5):483-485; Couetil, J. P., Argyriadis, P. G., Tolan, M. J., Achkar, A., and Carpentier, A. F. 2001. “Contralateral Lung Transplantation: A Left Lung Implanted in the Right Thorax,” Annals of Thoracic Surgery 72(3):933-955).

Another question pertained to whether the right lower lobe, a fifth again larger than that on the left, can be transplanted into the left thorax, and this too has been found to yield a good outcome. The finding is significant not only because a living donor can be used but because the size of the recipient can be less than that of the donor, thus materially increasing graft availability (Chen, F., Miyamoto, E., Takemoto, M., Minakata, K., Yamada, T., and 3 others 2015. “Right and Left Inverted Lobar Lung Transplantation,” American Journal of Transplantation 15(6):1716-1721).

Yet another question pertained to a combination of these-whether lobes from two different living donors would give good results in a donor of the same or smaller size, and this too was found acceptable (Kayawake, H., Chen-Yoshikawa, T. F., Motoyama, H., Hamaji, M., Hijiya, K., and 5 others 2018. “Inverted Lobes Have Satisfactory Functions Compared with Noninverted Lobes in Lung Transplantation,” Annals of Thoracic Surgery 105(4):1044-1049; Date, H., Aoyama, A., Hijiya, K., Motoyama, H., Handa, T., and 5 others 2017. “Outcomes of Various Transplant Procedures (Single, Sparing, Inverted) in Living-donor Lobar Lung Transplantation,” Journal of Thoracic and Cardiovascular Surgery 153(2):479-486; Chen, F., Kubo, T., Yamada, T., Sato, M., Aoyama, A., Bando, T., and Date, H. 2013. “Adaptation over a Wide Range of Donor Graft Lung Size Discrepancies in Living-donor Lobar Lung Transplantation,” American Journal of Transplantation 13(5):1336-1342).

Addressed above in section 2a, single lung transplantation is preferably from a living or a deceased donor on life support by extracorporeal membrane oxygenation and ventilator or direct stimulation of the respiratory centers in the brainstem through electrical or optogenetic stimulation and the circulatory center through the direct targeting of epinephrine or norepinephrine. To preclude numerous potential complications, a living donor is preferably anesthetized so as to minimize if not avoid the need for mechanical ventilation or circulatory life support during, not just as analgesia following surgery. A preferred method should allow a significant reduction if not the elimination of pain following as well as during the operation.

Methods for avoiding the use of general anesthesia and thus allow diaphragmatic contraction, hence, spontaneous breathing, or continuous spontaneous ventilation, have been developed in association with minimally invasive techniques such as endoscopic and robotic, which materially reduce the pain felt following such a procedure. In contrast, a thoracotomy requires not only pain suppression midprocedurally but postprocedurally when pain can be considerable. With additional local anesthesia, these methods should prove adaptable to the relatively short duration procedures described herein, as well as reduce the intensity of pain that follows a thoracotomy. Only in deceased donors is anesthesia noncritical.

General anesthesia, hence, diaphragmatic paralysis avoided, the chest dome can nevertheless pass through an endotracheal tube to deliver oxygen and an inhaled anesthetic if desired (see for example Allain, P. A., Carella, M., Agrafiotis, A. C., Burey, J., Assouad, J., and 5 others 2019. “Comparison of Several Methods for Pain Management after Video-assisted Thoracic Surgery for Pneumothorax: An Observational Study,” Online, BioMed Central Anesthesiology 19(1):120; Bang, S., Chung, K., Chung, J., Yoo. S., Baek, S., and Lee, S. M. 2019. “The Erector Spinae Plane Block for Effective Analgesia after Lung Lobectomy: Three Cases Report,” Medicine (Baltimore, Maryland) 98(29):e16262; Bang, S., Chung, J., Kwon, W., Yoo. S., Soh, H., and Lee, S. M. 2019 “Erector Spinae Plane Block for Multimodal Analgesia after Wide Midline Laparotomy: A Case Report,” Medicine (Baltimore, Maryland) 98(20):e15654; Chin, K. J. 2019. “Thoracic Wall Blocks: From Paravertebral to Retrolaminar to Serratus to Erector Spinae and Back Again-A Review of Evidence,” Best Practice and Research. Clinical Anesthesiology 33(1):67-77; Hong. B., Bang. S., Chung. W., Yoo. S., Chung, J., and Kim, S. 2019. “Multimodal Analgesia with Multiple Intermittent Doses of Erector Spinae Plane Block through a Catheter after Total Mastectomy: A Retrospective Observational Study,” Korean Journal of Pain 32(3):206-214; Kaushal. B., Chauhan, S., Saini, K., Bhoi. D., Bisoi, A. K., Sangdup. T., and Khan, M. A. 2019. “Comparison of the Efficacy of Ultrasound-guided Serratus Anterior Plane Block, Pectoral Nerves I I Block, and Intercostal Nerve Block for the Management of Postoperative Thoracotomy Pain after Pediatric Cardiac Surgery,” Journal of Cardiothoracic and Vascular Anesthesia 33(2):418-425; Kelsheimer, B., Williams, C., and Kelsheimer, C. 2019. “New Emerging Modalities to Treat Post-thoracotomy Pain Syndrome: A Review,” Missouri Medicine 116(1):41-44; Lan, L., Li, J., Xu X., and Cen, Y. 2019. “Lung Volume Reduction under Spontaneous Ventilation in a Patient with Severe Emphysema,” American Journal of Case Reports 20:125-130; Onishi, E., Toda, N., Kameyama, Y., and Yamauchi, M. 2019. “Comparison of Clinical Efficacy and Anatomical Investigation between Retrolaminar Block and Erector Spinae Plane Block,” Online, BioMed Research International 2019:2578396; Reyad, R. M., Shaker, E. H., Ghobrial, H. Z., Abbas, D. N., Reyad, E. M., and 3 others 2019. “The Impact of Ultrasound-guided Continuous Serratus Anterior Plane Block versus Intravenous Patient-controlled Analgesia on the Incidence and Severity of Post-thoracotomy Pain Syndrome: A Randomized, Controlled Study,” European Journal of Pain (London, England) August 21; Tsui, B. C. H., Fonseca, A., Munshey, F., McFadyen, G., and Caruso, T. J. 2019. “The Erector Spinae Plane (ESP) Block: A Pooled Review of 242 Cases,” Journal of Clinical Anesthesia 53:29-34; Urits, I., Charipova, K., Gress, K., Laughlin, P., Orhurhu, V., Kaye, A. D., and Viswanath, O. 2019. “Expanding Role of the Erector Spinae Plane Block for Postoperative and Chronic Pain Management,” Current Pain and Headache Reports 23(10):71; Yang, H.2. Dong, Q., Liang, L., Liu, J., Jiang. L., Liang. H., and Xu. S. 2019. “The Comparison of Ultrasound-guided thoracic Paravertebral Blockade and Internal Intercostal Nerve Block for Non-intubated Video-assisted Thoracic Surgery,” Journal of Thoracic Disease 11(8):3476-3481; Yeom, J. H. and Park, S. 2019. “Alternative Anesthetic Management of Video-assisted Thoracic Surgery for Spontaneous Breathing,” Korean Journal of Anesthesiology 72(2):196-197; Chakravarthy, M. 2018, Op cit. under section 2a; Goto, T. 2018. “What is the Best Pain Control after Thoracic Surgery?,” Journal of Thoracic Disease 10(3):1335-1338; Khoronenko, V., Baskakov, D., Leone, M., Malanova, A., Ryabov. A., Pikin, O., and Golovashchenko M. 2018. “Influence of Regional Anesthesia on the Rate of Chronic Postthoracotomy Pain Syndrome in Lung Cancer Patients,” Annals of Thoracic and Cardiovascular Surgery (Tokyo, Japan) 24(4):180-186; Saad, F. S., El Baradie, S. Y., Abdel Aliem, M. A. W., Ali, M. M., Kotb. T. A. M. 2018. Ultrasound-guided Serratus Anterior Plane Block versus Thoracic Paravertebral Block for Perioperative Analgesia in Thoracotomy,” Saudi Journal of Anaesthesia 12(4):565-570; Swisher, M. W., Gabriel, R. A., and Khatibi. B. 2018. “Two-Level Continuous Thoracic Paravertebral Nerve Blocks Providing Opioid-Free Postoperative Analgesia after Latissimus Dorsi Flap Breast Reconstruction: A Case Report,” Anesthesia and Analgesia Practice 11(5):118-120; Weinstein, E. J., Levene, J. L., Cohen, M. S., Andreae, D. A., Chao, J. Y., and 3 others 2018. “Local Anaesthetics and Regional Anaesthesia versus Conventional Analgesia for Preventing Persistent Postoperative Pain in adults and Children,” Cochrane Database of Systematic Reviews 4: CD007105; Yang, H. M., Choi, Y. J., Kwon, H. J., O. J., Cho. T. H., and Kim, S. H. 2018. “Comparison of Injectate Spread and Nerve Involvement between Retrolaminar and Erector Spinae Plane Blocks in the Thoracic Region: A Cadaveric Study,” Anaesthesia 73(10):1244-1250; Kachulis. B., Mitrev. L., and Jordan, D. 2017. “Intraoperative Anesthetic Management of Lung Transplantation Patients,” Best Practice and Research. Clinical Anesthesiology 31(2):261-272; Lenz, N., Hirschburger, M., Roehrig R., Menges, T., Mueller, M., Padberg, W., and Mann V. 2017. “Application of Continuous Wound-infusion Catheters in Lung Transplantation: A Retrospective Data Analysis,” Thoracic and Cardiovascular Surgeon (Stuttgart, Germany) 65(5):403-409; Park, S. Y. 2017. “Non-intubated Thoracic Surgery under Thoracic Epidural Anesthesia,” Korean Journal of Anesthesiology 70(3):235-236; Sunaga. H., Blasberg, J. D., and Heerdt. P. M. 2017. “Anesthesia for Nonintubated Video-assisted Thoracic Surgery,” Current Opinion in Anesthesiology 30(1):1-6; Tahara, S., Inoue, A., Sakamoto, H., Tatara, Y., Masuda, K., and 4 others 2017. “A Case Series of Continuous Paravertebral Block in Minimally Invasive Cardiac Surgery,” Journal of Anesthesia Clinical Reports 3(1):45; Zheng, H., Hu, X. F., Jiang, G. N., Ding, J. A., and Zhu, Y. M. 2017. “Nonintubated-awake Anesthesia for Uniportal Video-assisted Thoracic Surgery Procedures,” Thoracic Surgery Clinics 27(4):399-406; Nicoara. A. and Anderson-Dam, J. 2017. “Anesthesia for Lung Transplantation,” Anesthesiology Clinics 35(3):473-489; Unkart, J. T., Padwal, J. A., Ilfeld, B. M., and Wallace, A. M. 2016. “Treatment of Post-Latissimus Dorsi Flap Breast Reconstruction Pain with Continuous Paravertebral Nerve Blocks: bA Retrospective Review,” Anesthesiology and Pain Medicine 6(5):e39476; Yeung, J. H., Gates, S., Naidu, B. V., Wilson, M. J., and Gao Smith, F. 2016. “Paravertebral Block versus Thoracic Epidural for Patients Undergoing Thoracotomy,” Cochrane Database of Systematic Reviews 2: CD009121; Duggappa, D. R., Rao, G. V., and Kannan, S. 2015. “Anaesthesia for Patient with Chronic Obstructive Pulmonary Disease,” Indian Journal of Anaesthesia 59(9):574-583; Kiss, G. and Castillo, M. 2015. “Non-intubated Anesthesia in Thoracic Surgery: Technical Issues,” Annals of Translational Medicine 3(8):109; Kiss, G. and Castillo, M. 2015. “Nonintubated Anesthesia in Thoracic Surgery: General Issues,” Annals of Translational Medicine 3(8):110; Carmona, P., Llagunes, J., Casanova, I., Mateo, E., Canovas, S., and 4 others 2012. “Continuous Paravertebral Analgesia versus Intravenous Analgesia in Minimally Invasive Cardiac Surgery by Mini-thoracotomy,” (in Spanish with English abstract at Pubmed), Revista Española de Anestesiologia y Reanimación [Spanish Journal of Anesthesiology and Resuscitation] (Barcelona, Spain) 59(9):476-482; Warner, D. O. 2002. “Diaphragm Function during Anesthesia: Still Crazy after All These Years,” Anesthesiology 97(2):295-297).

The donor is prepared with solenoid-valved bronchus-to bronchus, or bronchial air-line and servovalve bloodlines so that the distal ends of the lines come off in the direction of the donor lung to be replaced. As specified in section 2b(1)(a), entitled Sudden Compound Vascular Bypass, or Sudden Switched-organ, Transplantation, the lines are made of the expandable clot and biofilm fabric to be removed when donor and recipient ipsilateral hilar ductus-main bronchus, pulmonary artery, pulmonary veins, and azygos vein—are anastomosed. The living or deceased donor or donors, having been supported as delineated in section 2a should be prepared in advance. In a same-side lung transplant, the recipient is placed beside the donor side-by-side, head-to-toe, the organ then rotated to the upright position when transferred.

Lung transplantation with as little circulatory or respiratory interruption as possible a central object, to minimize lung collapse, lung transplants should be performed with both donor and recipient under chest domes, as addressed above in section 2a, entitled Background of the Invention, and below in sections 2b(2)(g), entitled Ischemia and Hypoperfusion-free Carotid Endarterectomy Bypass and Prosthesis, and 5, entitled Description of the Drawing Figures.

Brief lung collapse during lung surgery, or surgical pneumothorax, is an accepted practice in single lung ventilation, and collapse during the much briefer interval required to transfer the harvested lung from the chest dome of the donor to that of the recipient is incapable of producing medically significant hypoxia, hypercapnia, or hypoxic pulmonary vasoconstriction (see, for example, David, P., Pompeo, E., Fabbi, E., and Dauri, M. 2015, Op cit., section 2a).

Positioning thus allows the use of shorter air and bloodlines. Preparation of both donor or donors and recipient is performed with the aid of a thorough checklist. The risk of errors due to the reversal in direction are averted by color coding like vessels in the donor or donors and recipient. Opposite side or contralateral lung transplantation is with donor and recipient side-by-side and head-to-head. When one same-side lung each is to be transplanted from two living or deceased, or one living and the other deceased donors, the head-to-toe arrangement applies to the donors to either side of the recipient. When both lungs are taken from the same deceased donor, the table is rotated; that is, the arrangement is side-by-side, head-to-toe.

Replacement of both lungs can be simultaneous, with one donor to either side of the recipient. With two living, two deceased, or one living and one dead donor, bilateral transplantation can be simultaneous or sequential. From a deceased donor, both lungs are harvested with the trachea, but not, as in a conventional double lung transplant, separated. Uninterrupted spontaneous breathing desired throughout the procedure, to prevent a midprocedural pneumothorax, both donor and recipient are hemiclamshell thoracotomized with retractors under chest domes addressed in sections 2a, 2b(2)(g), and 6.

Single lung respiration under artificial ventilation is performed on the donor, with the recipient breathing spontaneously, or if necessary, with the aid of a ventilator (see, for example, Kremer, R., Aboud, W., Haberfeld, O., Armali, M., and Barak, M. 2019. “Differential Lung Ventilation for Increased Oxygenation during One Lung Ventilation for Video Assisted Lung Surgery,” Journal of Cardiothoracic Surgery 14(1):89).

In addition to the advantages of regional rather than general anesthesia and uninterrupted nonfluctuating normal rate and pressure perfusion and aeration throughout the operation, serious sequelae associated with changes in blood pressure, also seen in cardiac transplantation, are avoided (see, for example, Mailhot, T., Cossette, S., Lambert, J., Beaubien-Souligny, W., Cournoyer, A., and 8 others 2019. “Delirium after Cardiac Surgery and Cumulative Fluid Balance: A Case-control Cohort Study,” Journal of Cardiothoracic and Vascular Anesthesia 33(1):93-101; Nguyen, D. N., Huyghens, L., Parra, J., Schiettecatte, J., Smitz, J., and Vincent, J. L. 2018. “Hypotension and a Positive Fluid Balance are Associated with Delirium in Patients with Shock,” Online, PLoS [Public Library of Science] One 13(8):e0200495; Anderson, B. J. and Diamond, J. M. 2016. “Under Pressure: Reduced Cerebral Perfusion as a Risk Factor for Postoperative Delirium in Lung Transplant Recipients,” Annals of the American Thoracic Society 13(2):156-157; Smith, P. J., Blumenthal, J. A., Hoffman, B. M., Rivelli, S. K., Palmer, S. M., Davis, R. D., and Mathew, J. P. 2016. “Reduced Cerebral Perfusion Pressure during Lung Transplant Surgery is Associated with Risk, Duration, and Severity of Postoperative Delirium,” Annals of the American Thoracic Society 13(2):180-187).

Accordingly, an effort is made to stabilize cerebral pressure throughout. In the absence of innervation, the implant response system would still monitor and adjust the component physiological, or nonpsychiatric, factors or indicia associated with postoperative delirium, such as pressure, rate, and composition of fluid delivery. Subtle changes in breathing rate such as due to intense emotion or exercise would still benefit from reinnervation. Given that to do so adds time to the procedure, whether to anastomose the pulmonary plexus, comprising branches of the parasympathetic vagus and sympathetic chain, in an attempt to reinnervate it must remain a clinical judgment.

While spontaneous local function is interrupted if not lost, various strategies exist to reinstate such function. In the living, this includes nerve allografts (see, for example, Csobonyeiova, M., Polak, S., Zamborsky, R., and Danisovic, L. 2019. “Recent Progress in the Regeneration of Spinal Cord Injuries by Induced Pluripotent Stem Cells,” Online, MDPI [Multidisciplinary Digital Publishing Institute] International Journal of Molecular Sciences (Basel, Switzerland) 20(15). pii: E3838; Gong, Z., Xia, K., Xu, A., Yu, C., Wang, C., and 5 others 2019. “Stem Cell Transplantation: A Promising Therapy for Spinal Cord Injury,” Current Stem Cell Research and Therapy August 23; Kou, Y. H., Yu, Y. L., Zhang, Y. J., Han, N., Yin, X. F., and 5 others 2019. “Repair of Peripheral Nerve Defects by Nerve Transposition Using Small Gap Bio-sleeve Suture with Different Inner Diameters at Both Ends,” Neural Regeneration Research 14(4):706-712; Yu, F., Yu, Y. L., Niu, S. P., Zhang, P. X., Yin, X. F., and 5 others 2019. “Corrigendum: Repair of Long Segmental Ulnar Nerve Defects in Rats by Several Different Kinds of Nerve Transposition,” Neural Regeneration Research 2019 14(7):1137; Yu, C., Xia, K., Gong, Z., Ying, L., Shu, J., and 4 others 2019. “The Application of Neural Stem/Progenitor Cells for Regenerative Therapy of Spinal Cord Injury,” Current Stem Cell Research and Therapy 14(6):495-503; Yu, F., Yu, Y. L., Niu, S. P., Zhang, P. X., Yin, X. F., and 5 others 2019. “Repair of Long Segmental Ulnar Nerve Defects in Rats by Several Different Kinds of Nerve Transposition,” Neural Regeneration Research 14(4):692-698; Yuan, Y. S., Niu, S. P., Yu, Y. L., Zhang, P. X., Yin, X. F., and 6 others 2019. “Reinnervation of Spinal Cord Anterior Horn Cells after Median Nerve Repair Using Transposition with Other Nerves,” Neural Regeneration Research 14(4):699-705; Gong, L., Jiang, C., Liu, L., Wan, S., Tan, W., and 9 others 2018. “Transfection of Neurotrophin-3 into Neural Stem Cells Using Ultrasound with Microbubbles to Treat Denervated Muscle Atrophy,” Experimental and Therapeutic Medicine 15(1):620-626; Nagoshi, N. and Okano, H. 2018. “iPSC [induced pluripotent stem cells]-derived Neural Precursor Cells: Potential for Cell Transplantation Therapy in Spinal Cord Injury,” Cellular and Molecular Life Sciences 75(6):989-1000; Khazaei, M., Siddiqui, A. M., and Fehlings, M. G. 2014. “The Potential for iPS [induced pluripotent]-derived Stem Cells as a Therapeutic Strategy for Spinal Cord Injury: Opportunities and Challenges,” Online, MDPI [Multidisciplinary Digital Publishing Institute] Journal of Clinical Medicine (Basel, Switzerland) 4(1):37-65; Chao, T., Frump, D., Lin, M., Caiozzo, V. J., Mozaffar, T., Steward, O., and Gupta R. 2013. “Matrix Metalloproteinase 3 Deletion Preserves Denervated Motor Endplates after Traumatic Nerve Injury,” Annals of Neurology 73(2):210-223; Nogradi, A., Pajer, K., and Marton, G. 2011. “The Role of Embryonic Motoneuron Transplants to Restore the Lost Motor Function of the Injured Spinal Cord,” Annals of Anatomy 193(4):362-370).

Since one or more basic side-entry jackets with inmate or associated sensors can signal signs of rejection to the control microprocessor which responds by directly targeting immunosuppressants to the anastomoses, outcomes should prove more favorable (see, for example, Chong, T. 2011. “Discussion. Pathways of Sensory Recovery after Face Transplantation,” Plastic and Reconstructive Surgery 127(5):1890-1891; Siemionow, M., Gharb, B. B., and Rampazzo, A. 2011. “Pathways of Sensory Recovery after Face Transplantation,” Plastic and Reconstructive Surgery 127(5):1875-1889; Bregeon, F., Alliez, J. R., Hery, G., Marqueste, T., Ravailhe, S., and Jammes, Y. 2007. “Motor and Sensory Re-innervation of the Lung and Heart after Re-anastomosis of the Cervical Vagus Nerve in Rats,” Journal of Physiology 2007 581 (Part 3):1333-1340; Dubernard, J. M., Lengele, B., Morelon, E., Testelin, S., Badet, L., and 22 others 2007. “Outcomes 18 Months after the First Human Partial Face Transplantation,” New England Journal of Medicine 357(24):2451-2460; Guntinas-Lichius, O. 2008. “Outcomes 18 Months after the First Human Partial Face Transplantation,” New England Journal of Medicine 358(20):2179-2180).

Both donor and recipient hemithoracotomized, the lung of each for transplant is pulled laterally to expose the hilar ipsilateral pulmonary arteries, pulmonary veins, and bronchus. Regardless of the method used, in almost one half of lung transplant recipients, denervation bodes some adverse consequences (see, for example, (see, for example, Anderson, B. J., Chesley, C. F., Theodore, M., Christie, C., Tino, R., and 10 others 2018. “Incidence, Risk Factors, and Clinical Implications of Post-operative Delirium in Lung Transplant Recipients,” Journal of Heart and Lung Transplantation (New York, New York) 37(6):755-762; Smith, P. J., Stonerock, G. L., Ingle, K. K., Saulino, C. K., Hoffman, B., and 7 others 2018. “Neurological Sequelae and Clinical Outcomes after Lung Transplantation,” Transplantation Direct 4(4):e353; Gamez, J., Salvado, M., Martinez-de La Ossa, A., Deu, M., Romero, L., and 10 others 2017. “Influence of Early Neurological Complications on Clinical Outcome following Lung Transplant,” Online, PLoS [Public Library of Science] One 12(3):e0174092; Mateen, F. J., Dierkhising, R. A., Rabinstein, A. A., van de Beek, D., and Wijdicks. E. F. 2010. “Neurological Complications following Adult Lung Transplantation,” American Journal of Transplantation 10(4):908-914; Ferdinande. P., Bruyninckx, F., Van Raemdonck, D., Daenen, W., and Verleden, G. 2004. “Phrenic Nerve Dysfunction after Heart-Lung and Lung Transplantation,” Journal of Heart and Lung Transplantation (New York, New York) 23(1):105-109).

Any significant indication of rejection detected by the implant sensor and responded to by the direct targeting to the anastomoses by the automatic response system implanted in the recipient, unless the donor lung presents signs or symptoms of impairment, ex vivo lung perfusion is dispensed with. To assure sufficient stump length for a secure switch with adequate room to achieve secure anastomoses, the donor ductus, that is, pulmonary vessels and bronchus, have been servovalve-jacketed as close to the parenchyma as avoids dissection into and trauma to the replacement organ. While the length of the stumps in either lung might appear not to furnish the length required, combining both with added length gained by cut down exposure of those from the native lung will provide stumps of sufficient length.

Depending upon the relative size of the organs, the length of the recipient ductus will usually be greater. An automatic disorder detection and response system is not implanted in a living donor, who can receive unimposing preprocedural tolerance induction treatment and report any pertinent symptoms. In contrast, as well as conventionally, such as by exchange of blood products, the deceased donor has been monitored and treated with the aid of such a system. When harvested, the sensors, hence the accessory channels, associated with the donor, removed from their physiological milieu, no longer provide relevant data and responses, so that these electrical and medicinal lines, or sidelines, are disconnected, the servovalve jacket with only the mainlines, or bloodlines connected, to the corresponding ductus in the recipient.

The servovalve-jackets in the recipient, which will administer the metered tolerance induction consistent with the gradual transfer of the lung from the circulatory system of the donor into that of the recipient, will remain with accessory channels and other system componentry indefinitely to monitor and release immunosuppressant, anti-inflammatory, and antimicrobial medication by directly targeting the anastomoses through the jackets. Both donor and recipient lungs still integral in their respective positions, the mainline connected to each ductus in the donor is connected to the corresponding or counterpart ductus in the recipient, and the process of gradually replacing the blood flowing through the lung with that of the recipient initiated.

Once the blood is entirely that of the recipient, plus an additional interval if prescribed by the immunology staff, recipient and donor lungs are harvested, or pneumonectomized, by transection of their ductus, and the donor organ placed in the position previously occupied by the native organ of the recipient. Because the pleural cavities, or pleural spaces, afford little unaccounted for space, and the intentional transplantation of an undersized or lobar transplant invites less propitious outcomes than does an intact lung, rather than to retain the mainline connections without anastomosis, the anastomoses are completed.

Each mainline now bypasses, that is, circles around its respective anastomosis from the recipient to the donor jacket. The anastomoses confirmed to be free of leaks, the mainlines and donor jackets are removed, while the recipient jackets are retained for their accessory channels. Ancillary medical considerations and minor alternatives as to pre- or postbifurcation jacketing are clearly described and illustrated by Hayanga and Cunha, Op cit., this section. To avert the need for surgical measures, the implanted system can directly and automatically treat gastric hyperacidity by releasing antacids, and should a surgical procedure done earlier remain intact or failed, the system can support the surgical site with anti-inflammatoires and antimicrobials, for example.

As to the capability of the implanted automatic disorder response system, following its administration of a lung transplant, it can be used to automatically release an antiviral, for example, in adherence to its prescription program. The relations of lung transplantation to gastroesophageal reflux disease and gastroparesis—that it interferes with breathing-predisposes to chronic lung allograft dysfunction and bronchiolitis obliterans. [perhaps in combination with cytomegalovirus infection (Piloni, D., Gabanti, E., Morosini, M., Cassinelli, G., Frangipane. V., and 7 others 2022. “Fifteen-Year Surveillance of LTR [lung transplant recipient] Receiving Pre-Emptive Therapy for CMV [cytomegalovirus] Infection: Prevention of CMV Disease and Incidence of CLAD [chronic lung allograft dysfunction],” MDPI [Multidisciplinary Digital Publishing Institute] Microorganisms (Basel, Switzerland) 10(12):2339; Hodson, E. M., Ladhani, M., Webster, A. C., Strippoli, G. F., and Craig, J. C. 2013. “Antiviral Medications for Preventing Cytomegalovirus Disease in Solid Organ Transplant Recipients,” Online, Cochrane Database of Systematic Reviews (Chichester, West Sussex, England)(2):CD003774; Manuel O, Kralidis G, Mueller N J, Hirsch H H, Garzoni C. and 8 others 2013. “Swiss Transplant Cohort Study. Impact of Antiviral Preventive Strategies on the Incidence and Outcomes of Cytomegalovirus Disease in Solid Organ Transplant Recipients,” Online, American Journal of Transplantation (New York, New York) 13(9):2402-2410; Hussain, S. and Singh, 2002. “Bronchiolitis Obliterans and Lung Transplantation: Evidence for an Infectious Etiology,” Seminars in Respiratory Infection 17(4):310-314), or that microaspiration of an acidic aerosol due to gastroesophageal reflux causes bronchiolitis (see, for example, Khoma, O., Park, J. S., Lee, F. M., Van der Wall, H., and Falk, G. L. 2022. “Different Clinical Symptom Patterns in Patients with Reflux Micro-aspiration,” Online, European Respiratory Journal Open Research (Sheffield, England) 8(1):00508-2021; or some other cause related or not related ((see, for example Jadcherla, A. V., Litzenberg, A. V., and Balasubramanian, G. 2022. “Esophageal Dysfunction in Post-lung Transplant: An Enigma,” Dysphagia (New York, New York)(38(3):731-743; or immune injury (Al-Githmi, I., Batawi, N., Shigemura, N. Hsin, M., Lee, T. W. He. G. W., and Yim, A. “Bronchiolitis Obliterans following Lung Transplantation,” Online, European Journal of Cardiothoracic Surgery 30(6):846-851)] has been alluded to in preceding sections, and can be treated with the aid of an implanted automatic disorder control system.

The relation of acid reflux to graft failure following lung transplantation has been under study since the turn of the century (see, for example, Ichkhanian Y., Hwang, J. H., Ofoso, A. Li, A. A., Szvarca, D., and 7 others 2022. “Role of Gastric Per-oral Endoscopic Myotomy (G-POEM) in Post-lung Transplant Patients: A Multicenter Experience,” Online, Endoscopy International Open 10(6):E832-E839; Varma, S., Yun, J. H., Kim, J. S., Podolanczuk, A. J., Patel, N. M., and Bernstein, E. J. 2022. “Clinical Characteristics Associated with Small Airways Diease in Systemic Sclerosis,” Journal of Scleroderma and Related Disorders 7(2):128-134; Bedair, B. and Hachem, R. R. 2021. “Management of Chronic Rejection after Lung Transplantation,” Online, Journal of Thoracic Disease 13(116645-6653; Wong, D. and Chan, W. W. 2021. “Foregut Dysmotility in the Lung Transplant Patient,” Current Gastroenterology Reports 23(12):23; Hathorn, K. E., Chan, W. W., and Lo, W. K. 2017. “Role of Gastroesophageal Reflux Disease in Lung Transplantation,” Online, World Journal of Transplantation (Pleasanton, California) 7(2):103-116; Davis et al. 2010. “Gastroesophageal Reflux Disease after Lung Transplantation: “Pathophysiology and Implications for Treatment,” Online, Surgery 148(4):737-745. Parada, M. T., Alba, A. and Sepulveda, C. 2010. “Bronchiolitis Obliterans Syndrome Development in Lung Transplantation Patients,” Transplantation Proceedings 42(1):331-332; King. B. J., Iyer. H., Leidl. A. A., and Carby, M. R. 2009. “Gastroesophageal Reflux in Bronchiolitis Obliterans Syndrome: A New Perspective,” Journal of Heart and Lung Transplantation (New York, New York) 28(8):870-875; Davis, R. D., Jr., Lau C. L., Eubanks, S., Messier, R. H., Hadjiliadis, D., Steele, M. P., and Palmer, S. M. 2003. “Improved Lung Allograft Function after Fundoplication in Patients with Gastroesophageal Reflux Disease Undergoing Lung Transplantation,” Online, Journal of Thoracic and Cardiovascular Surgery 125(3):533-542).

Surgical methods to eliminate gastroesophageal reflux therefore include implantation of an electrostimulator, repair of a hiatal, or hiatus, hernia by means of a Nissen or Toupe esophagogastric fundoplication (Seeras, K., Bittar, K., and Siccardi, M. A. 2022. “Nissen Fundoplication,” Online, Treasure Island, Florida: StatPearls Publishing Co.; Yadlapati, R. Hungness, E. S. and Pandolfino, J. E. 2018. “Complications of Antireflux Surgery,” Online, American Journal of Gastroenterology 113(8):1137-1147), magnetic bead lower esophageal sphincter constraint bracelet, esophagectomy to remove an esophageal obstructon caused by gastric acid reflux, caustic ingestion such as of strongly corrosive alkalis, or a benign tumor (see, for example, Mormando, J., Barbetta, A., and Molena, D. 2018. “Esophagectomy for Benign Disease,” Online, Journal of Thoracic Disease,” 10(3):2026-2033); esophagectomy to reverse a failed earlier fundoplication (Shen, K. R., Harrison-Phipps, K. M., Cassivi, S. D., Wigle, D., and Nichols, F. C. III, and 3 others 2010. “Esophagectomy after Anti-reflux Surgery,” Online, Journal of Thoracic and Cardiovascular Surgery 139(4):969-975), and transoral incisionless fundoplication; the Stretta radiofrequency heat treatment procedure, having proven less successful (see, for example, Stanak, M., Erdos, J., Hawlik, K., and Birsan, T. 2018. “Novel Surgical Treatments for Gastroesophageal Reflux Disease: Systematic Review of Magnetic Sphincter Augmentation and Electric Stimulation Therapy,” Gastroenterology Research 11(3):161-173; Mermelstein, J., Chait Mermelstein, A., and Chait, M. M. 2018. “Proton Pump Inhibitor-refractory Gastroesophageal Reflux Disease: Challenges and Solutions,” Clinical and Experimental Gastroenterology 11:119-134.

Pharmaceutical treatment is usually with an oral antacid such as sodium bicarbonate or calcium carbonate, a proton pump inhibitor, histamine h2 receptor antagonist, or alumina-magnesium suspension. Gamma-aminobutyric acid analog R-baclofen oral functions as a vagal nuclei inhibitory neurotransmitter, reducing the incidence of transient lower esophageal sphincter relaxations as the major cause of reflux (see, for example, Sidhu, A. S. and Triadafilopoulos, G. 2008. “Neuro-regulation of Lower Esophageal Sphincter Function as Treatment for Gastroesophageal Reflux Disease,” World Journal of Gastroenterology 14(7):985-990)(see also Aktar, R., Peiris, M., Fikree, A., Eaton, S., Kritas, S., and 7 others 2019. “A Novel Role for the Extracellular Matrix Glycoprotein-Tenascin-X in Gastric Function,” Journal of Physiology 597(6):1503-1515; Clarke, J. O., Fernandez-Becker, N. Q., Regalia, K. A., and Triadafilopoulos, G. 2018. “Baclofen and Gastroesophageal Reflux Disease: Seeing the Forest through the Trees,” Clinical and Translational Gastroenterology 9(3):137; Mermelstein, J., Chait Mermelstein, A., and Chait, M. M. 2018, Op cit., perceding paragraph; Sharma, N. and Anderson, S. H. C. 2013. “The Relevance of Transient Lower Oesophageal Sphincter relaxations in the Pathophysiology and Treatment of GORD,” Frontline Gastroenterology 4(3):171-174; Koek, G. H., Sifrim, D., Lerut, T., Janssens, J., and Tack, J. 2003. “Effect of the GABA (B) Agonist Baclofen in Patients with Symptoms and Duodeno- gastro-oesophageal Reflux Refractory to Proton Pump Inhibitors,” Gut 52(10):1397-1402; Zhang, Q., Lehmann, A., Rigda, R., Dent, J., and Holloway, R. H. 2002. “Control of Transient Lower Oesophageal Sphincter Relaxations and Reflux by the GABA (B) Agonist Baclofen in Patients with Gastro-oesophageal Reflux Disease,” Gut 50(1):19-24; Holloway, R. H. 2001. “Systemic Pharmacomodulation of Transient Lower Esophageal Sphincter Relaxations,” American Journal of Medicine 111 Supplement 8A: 178S-185S). The anxiolytic buspirone and antiemetic granisetron are administered orally, and granisetron is additionally infusible or absorbed through a transdermal patch (see, for example, Chen, J. H. 2016. “Ineffective Esophageal Motility and the Vagus: Current Challenges and Future Prospects,” Clinical and Experimental Gastroenterology 9:291-299).

While a relatively simple surgical procedure that should eliminate the problem is to be preferred, in a patient less prepared to undergo another procedure in addition to a lung transplant, if the transplant was accomplished using the sudden or metered switch method, then these alternative approaches, involving additional expense if not surgery, can be eliminated. In conjunction with an organ transplant, here one or both lungs, the valve or servovalve-jackets respectively are routinely left connected to the common or right and left pulmonary arteries where the accessory channels can then be used by the implanted automatic response system to detect and respond to signs of rejection.

Rejection as signaled by the appearance of obliterative bronchiolitis (bronchiolitis obliterans, popcorn lung-distinct from bronchiolitis obliterans organizing pneumonia, since redesignated cryptogenic organizing pneumonia) is addressed below in section 2b(1)(b)(b9), entitled Single Living and Single or Double Deceased Donor Metered Compound Vascular Bypass, or Switched-organ, Lung Transplantation.

2b(1)(b)(b10). Metered Compound Vascular Bypass, or Switched-Organ Heart-Lung Transplantation

The advantages in directly transferring transplant organs from living or from donors having been continuously life-supported past death through the sudden, and particularly, through the metered switch technique to recipients include continuity of cardiorespiratory, endocrine, and metabolic function, and the elimination of a nonperfused condition, which upon reperfusion, results in microvascular injury. In some if not all cases, a deceased donor can be neurostimulated to breathe spontaneously, which continuity in gas exchange has preservative value. A heart-lung transplant performed using the metered switch method virtually eliminates problem bleeding in the recipient.

Because in switch transplantation, the heart-lung bloc is harvested only after blood flow has been diverted away from the vascular transections, the major complication of problem bleeding associated with harvesting of the donor bloc encountered in conventional heart-lung transplantation is eliminated. The deceased donor having been maintained on mechanical ventilator support, or as addressed in section 2a, central stimulation of the respiratory center in the brain since death, any significant interruption in ventilation during transfer of the bloc from the donor to the recipient is with continued or initiated ventilator support, the interval minimized by anastomosing the trachea first.

The heart and lungs are removed, close attention paid to avoiding injury to the vagus nerve, its superior and recurrent laryngeal branches, and the phrenic nerves. Removal and transplantation as a block with the heart connected to the lungs allows connection of the lungs through connection of the heart, eliminating the need to separately connect each lung (Mancini, M. C. 2017. “Heart-Lung Transplantation Technique,” Medscape Online at https://emedicine.medscape.com/article/429188-technique #c2; Garcia, J. P. and Shah, R. 2015. “Heart and Lung En Bloc Transplantation,” Part 1, Chapter 4, pages 31-42 in Mathisen, D. J. and Morse, C. R., Master Techniques in Surgery: Thoracic Surgery: Transplantation, Tracheal Resections, Mediastinal Tumors, Extended Thoracic Resections, Philadelphia, Pennsylvania: Wolters Kluwer; Huddleston, C. B. and Richey, S. R. 2014, Op cit. section 2a(3), Remotely Procured Graft Organ Preservation.

The metered switch method eliminates a multiplicity of anastomoses and the risk of leaks. Endocrine and metabolic function monitoring and supplementation is a subspecialty practiced by anesthesiologists and intensivists (Shetty and other references cited in section 2a). As explained in section 2a, the preservation of a graft organ in a virtually living condition, intact and orthotopically located in its own body, essentially reduces the problem of transplantation using the sudden switch technique to one of immune compatibility, and the metered switch method reduces this remaining problem as well, making the introduction of a graft organ into a different body as unprovocative and uneventful as possible.

As always using the switch method, the graft organ or organs are harvested only when all or most of the blood that passes through these is that of the recipient. Such an unconventional eventuality is made possible by vascular valves and servovalves. The object is for each organ to function continuously throughout the transplantation procedure as if ‘nothing happened.’ The organs are therefore provided in optimal condition with the least trauma to the recipient.

With the aid of an artificial ventilator, or preferably electrostimulation of the pontine and medullary dorsal and ventral respiratory groups by means of stereotactically positioned needle electrodes, the deceased donor can be made to inhale and exhale through the lungs. However, whenever the chest is breached, the immediate entry of air at the higher pressure of the atmosphere, forces the lungs to collapse. This is counteracted through the use of a surgical chest dome, addressed in sections 2b(2)(k), entitled Surgical Chest Dome, 2a, Background of the Invention. General, and 2b(2)(g), entitled Ischemia and Hypoperfusion-free Carotid Endarterectomy Bypass and Prosthesis, and 2d, entitled Body Surface Side-entry Ports.

With the metered technique, extending the transfer over an interval makes it possible to accomplish a measure of immune tolerance induction as integral in the process through which the organ is transferred. Awake, or spontaneous breathing, the avoidance of general anesthesia, uninterrupted circulation, and immune tolerance induction concurrent with transfer from the donor to the recipient can reasonably be expected to increase the number of favorable outcomes. Transplantation thus requires, and so is made possible, by vascular valves for sudden, and servovalves for metered, switch transplantation. A heart-lung transplant requires only the aorta and venae cavae of donor and recipient to be diversion valve jacketed and connected.

Without the need to expose, cut back to lengthen, and jacket each vessel stump of the recipient at the hilum, then connect the mainline, or bloodline from each valve to its respective corresponding stump in the donor, a heart-lung transplant using the metered switch method is less complicated, and barring unforeseen eventualities, takes less time than to transplant a single lung. The same is no less true of the equivalent conventional procedures (Huddleston, C. B. and Richey, S. R. 2014, Op cit. under section 2a(3), Remotely Procured Graft Organ Preservation.

2b(1)(b)(b11). Metered Compound Vascular Bypass, or Switched-Organ Heterotopic Intact Allograft Thyroid and Parathyroid Transplantation

Autotransplantation of thyroid following thyroidectomy for benign multinodular goiter with increased risk of cancer is by injection of a minced, or diced, portion of the excised gland into muscle, usually the sternocleidomastoideoch has generally provided good outcomes (Smith, J. J., Chen, X., Schneider, D. F., Broome, J. T., Sippel. R. S., Chen, H., and Solórzano, C. C. 2013. “Cancer after Thyroidectomy: A Multi-institutional Experience with 1,523 Patients,” Journal of the American College of Surgeons 216(4):571-579; Smith, J. J., Chen, X., Schneider, D. F., Nookala, R., Broome, J. T., and 3 others 2013. “Toxic Nodular Goiter and Cancer: A Compelling Case for Thyroidectomy,” Annals of Surgical Oncology 20(4):1336-1340). The small pieces are difficult to extract should the need arise, prompting a method for transplanting the thyroid with or without parathyroids as intact, albeit in a somewhat inferior position.

Exceptionally, when there is concern that some damage may have been done to the recurrent laryngeal nerve, for example, the addition of no greater a number of minced pieces of thyroid than would negate their easy recovery should the need arise along with calcium and vitamin D supplementation or parathyroid hormone-related peptide replacement therapy but be sufficient to overcome the risk of laryngospasm, vocal cord paralysis, respiratory muscle paralysis and asphyxia as serious complications is advisable (see, for example, Gurluler, E. 2024. “The Use of Superior Parathyroid Gland as an Anatomical Landmark in Identifying Recurrent Laryngeal Nerve during Total Thyroidectomy: A Prospective Single-surgeon Study,” Online, Annals of Surgical Treatment and Research (Seoul, South Korea) 106(2):63-67; Tabata, H., Takahoko, K., Luthe, S. K., Makino, H., and Iwasaki, H. 2024. “Laryngospasm Treated with Intramuscular Rocuronium in a Pediatric Patient without Intravenous Access: A Case Report,” Online, Cureus (Palo Alto, California) 16(3):e56620; Gurluler, E. 2023. “The Use of Zuckerkandl's Tubercle as an Anatomical Landmark in Identifying Recurrent Laryngeal Nerve and Superior Parathyroid Gland during Total Thyroidectomy: A Prospective Single-surgeon Study,” Online, Frontiers in Surgery (Lausanne, Switzerland) 10:1289941; Patra, A., Asghar, A., Chaudhary, P., and Ravi, K. S. 2023. “Identification of Valid Anatomical Landmarks to Locate and Protect Recurrent Laryngeal Nerve during Thyroid Surgery: A Cadaveric Study,” Surgical and Radiologic Anatomy (Berlin, Germany) 45(1):73-80; Jin, S. and Sugitani, I. 2021. “Narrative Review of Management of Thyroid Surgery Complications,” Online, Gland Surgery (Hong Kong, China) 10(3):1135-1146; Bai, Y., Jing. X. R., Xia, Y., and Tao, X. N. 2020. “Paroxysmal Laryngospasm: A Rare Condition that Respiratory Physicians Must Distinguish from Other Diseases with a Chief Complaint of Dyspnea,” Online, Canadian Respiratory Journal (Cairo, Egypt) 2451703; Wang. W., Sun, J., Tang. H., Gao, Y., Chen, S., Li, M., and Zheng, H. 2020. “Main Branch of ACN-to-RLN [the ansa cervicalis nerve to the recurrent laryngeal nerve] for Management of Laryngospasm Due to Unilateral Vocal Cord Paralysis,” Laryngoscope (Philadelphia, Pennsylvania) 130(10):2412-2419; Michelet D. Truchot J, Piot M A, Drummond D, Ceccaldi P F, and 3 others 2018. “Perioperative Laryngospasm Management in Paediatrics: A High-fidelity Simulation Study,” Online, BMJ [British Medical Journal] Simulation and Technology Enhanced Learning (London, Englan) 26:5(3):161-166; Uludağ, M., Tanal. M., and işgor. A. 2018. “A Review of Methods for the Preservation of Laryngeal Nerves during Thyroidectomy,” in English, Online, Şişli Etfal Hastanesi Tip Bülteni [Medical Bulletin of Şişli Elfal Children's Hospital] (Istanbul, Turkey) 52(2):79-91; Dedivitis, R. A., Aires, F. T., and Cernea, C. R. 2017. “Hypoparathyroidism after Thyroidectomy: Prevention, Assessment, and Management,” Current Opinion in Otolaryngology and Head and Neck Surgery (Philadelphia, Pennsylvania) 25(2): 142-146; Gavel, G. and Walker, R. W. M. 2014. “Laryngospasm in Anaesthesia,” Online, Continuing Education in Anaesthesia Critical Care and Pain (Oxford, England) 14(2):47-51; Huang. S. M. 2012. “Do We Overtreat Post-thyroidectomy Hypocalcemia?,” World Journal of Surgery (Hoboken, New Jersey) 36(7):1503-1508).

Thyroid hormone, that is, thyroxine, or T4, and triiodothyronine, or T3, are secreted continuously and have a short half-life, so that longer lasting synthetic replacements such as levothyroxine (L-thyroxine) are used. Apart from or following transplantation of the thyroid and/or parathyroid glands, or if necessary to reinstate or initiate normal function in native or transplanted glands, a fully implanted automatic disorder response system can simulate normal endocrine function by continuously monitoring the sufficiency of and administer the release of replacement synthetic or synthetics to satisfy the deficit to the extent needed to maintain levels within the normal ranges.

Distinguished as primary, central, or congenital, hypothyroidism, or hypothyreosis, each form of the disorder produces a variety of symptoms of which the implanted system should treat those amenable to substance monitoring and release. Primary hypothyroidism requires sensor monitoring of and the automated release of supplemental iodine, while the central form involves structural defects that necessitate surgery prior to the fitting of an automatic response system, that congenital often requiring attention to impairments in the special senses. Hypothyroidism can be a devastating disorder affecting multiple organs and tissues. Given this profusion of variables and sequelae, the treatment of hypothyroidism can involve numerous and diverse impairments as to defy neat summarization and appropriately recommend a responsive system under hierarchical control.

Precursory technology to develop implantable sensors to detect the serum levels of thyroid-related hormones already exists (see, for example, Singh, D., Bharti, A., and Mathur, A. 2023. “Thyroxine Biosensors: A Review on Progress and Challenges,” Bioanalysis (London, England) 15(20):1259-1270; Liu, J., Yu. C., Han, L., Shen, Y., Fang. Y., and 7 others 2022. “Upconversion Luminescence-based Aptasensor for the Detection of Thyroid-stimulating Hormone in Serum,” Mikrochimica Acta (Vienna, Austria) 189(5):179; Asav. E. 2021. “Development of a Functional Impedimetric Immunosensor for Accurate Detection of Thyroid-stimulating Hormone,” Online, Turkish Journal of Chemistry (Ankara, Turkey) 45(3):819-834; Özcan, H. M. and Aydin, U. D. 2021. “A Simple Immunosensor for Thyroid Stimulating Hormone,” Online, Artificial Cells, Nanomedicine, and Biotechnology (Oxford, England) 49(1):61-70; Bendo, L., Casanova, M., Figueira, A. C., Polikarpov, I., and Zucolotto, V. 2014. “Nanostructured Sensors Containing Immobilized Nuclear Receptors for Thyroid Hormone Detection,” Online, Journal of Biomedical Nanotechnology (Stevenson Ranch, California) 10(5):744-750):Wagner R L, Huber B R. Shiau A K, Kelly A, Cunha Lima S T. and 5 others 2001. “Hormone Selectivity in Thyroid Hormone Receptors,” Online, Molecular Endocrinology (Chevy Chase, Maryland) 15(3):398-410).

Similarly, precursory technology to develop implantable sensors to detect the serum levels of parathyroid hormone (parathormone or parathyrin) already exists, so that an implanted automatic disorder response system can be enabled to detect and if necessary, ameliorate an insufficiency of parathyroid hormone as needed to bring the level to within the normal range (see, for example, Nie. Y., Li, J., Liu, Y., Zhang, Q., and Ma, Q. 2020. “A Visual FRET [Fluorescence Resonance Energy Transfer] Immunofluorescent Biosensor for Ratiometric [system whereby an output is directly proportional to the input] Parathyroid Hormone (1-84) [see Cusano et al., 2015 following] Antigen Point-of-care Detection,” Online, Journal of Fluorescence (Amsterdam, North Holland, Netherlands) 30(2):329-334; Cusano, N. E., Rubin, M. R., and Bilezikian, J. P. 2015. “PTH (1-84) Replacement Therapy for the Treatment of Hypoparathyroidism,” Online, Expert Review of Endocrinology and Metabolism (London, England) 10(1):5-13; Özcan, H. M and Sezginturk, M. K. 2015. “Detection of Parathyroid Hormone Using an Electrochemical Impedance Biosensor Based on PAMAM [poly amidoamine] Dendrimers,” Biotechnology Progress (Hoboken, New Jersey) 31(3):815-822; Özcan, H. M., Yildiz, K., çakar, C., Aydin. T., Asav. E., Sağiroğlu, A., and Sezgintürk, M. K. “Ultrasensitive Impedimetric Biosensor Fabricated by a New Immobilisation Technique for Parathyroid Hormone,” Applied Biochemistry and Biotechnology (Clifton, New Jersey) 176(5):1251-1262; SayιklιŞimşek, C., Nur Sonuç Karaboğa, M., and Sezgintürk, M. K. 2015. “A New Immobilization Procedure for Development of an Electrochemical Immunosensor for Parathyroid Hormone Detection Based on Gold Electrodes Modified with 6-Mercaptohexanol and Silane,” Talanta (Amsterdam, North Holland, Netherlands) 144:210-218).

Moreover, precursory technology to develop implantable sensors to detect endocrine disrupting chemicals in the blood and urine already exists (see, for example, Shah, M. M., Ahmad, K., Boota, S., Jensen, T., La Frano, M. R., and Irudayaraj. J. 2023. “Sensor Technologies for the Detection and Monitoring of Endocrine-disrupting Chemicals,” Online, Frontiers in Bioengineering and Biotechnology (Lausanne, Switzerland) 11:1141523; Calsolaro. V., Pasqualetti, G., Niccolai, F., Caraccio, N., and Monzani, F. 2017. “Thyroid Disrupting Chemicals,” Online, MDPI [Multidisciplinary Digital Publishing Institute] International Journal of Molecular Sciences (Basel, Switzerland 18(12):2583).

An artificial thyroid gland would evade the risk of immune rejection, ischemia, and infection to leave a predictable immune foreign body reaction, but the need for such a device assumes that, improbably, the innate organ were completely missing or had been completely destroyed. However, given the conventional use of small fragments rather than an intact gland anyway and the advent of immunosuppressants, an artificial thyroid gland would have utility if and only if no alternative source of graft tissue was unavailable (see, for example, Ogundipe, V. M. L., Plukker, J. T. M., Links, T. P., and Coppes. R. P. 2022. “Thyroid Gland Organoids: Current Models and Insights for Application in Tissue Engineering,” Tissue Engineering Part A (New Rochelle, New York) 28(11-12):500-510; Li, L., Sheng, Q., Zeng, H., Li, W., Wang, Q., and 4 others 2022. “Engineering a Functional Thyroid as a Potential Therapeutic Substitute for Hypothyroidism Treatment: A Systematic Review,” Online, Frontiers in Endocrinology (Lausanne, Switzerland) 13:1065410).

Implantation in the form of small pieces rather than an intact thyroid were it to transition to malignancy or if allogeneic, rejection makes recovery of the implant more difficult under exigent circumstances. Allotransplantation following thyroid removal for cancer where a recrudescence and/or rejection is a possibility, however, is best of the gland as continuous tissue to a location that will later permit ready access. Optogenetic methods can be used to facilitate recovery of the bits, but only at the expense of extending procedural time. Here thyroid transplantation is of an intact thyroid gland taken and transferred from the circulatory system of the donor while remaining on life support as would any other organ by metered compound vascular bypass transplantation.

Conventionally, the parathyroids like the thyroid can be resituated in the body intact or in particles, opening the possibility of errors in treatment or considerable difficulty in recovery should the patient later be brought unconscious to the emergency room with a need to locate and remove the resituated glands, especially if fragmented (see, for example, Aysan, E. Altug, B., Ercan, C., Toka, C. K., Idiz, U. O., and Muslumanoglu, M. 2016. “Parathyroid Allotransplant with a New Technique: A Prospective Clinical Trial,” Experimental and Clinical Transplantation 14(4):431-435; Cavallero, G., Iorio, O., Centanni, M., Porta, N., Gargano, L., and 4 others 2015. “Parathyroid Reimplantation in Forearm Subcutaneous Tissue during Thyroidectomy: A Simple and Effective Wau to Avoid Hypoparathyroidism,” World Journal of Surgery 39(8):1936-1942; Tan, C. C., Cheah, W. K., Tan, C. T. K., and Rauff. A. 2010. “Intramuscular Injection of Parathyroid Autografts is a Viable Option after Total Parathyroidectomy,” World Journal of Surgery 34(6):1332-1336).

Usually autotransplanted, if necessary, recovery of a fragmented gland if allotransplanted can be more serious. Disintegrated thus, any condition, such as a malignant transition or rejection which necessitated the recovery of the transplanted cells would necessitate dissection and excision of far wider extent than had the glands been left intact. Parathyroid cell suspensions could pose a more serious complication were the cells to be rejected or become malignant thus affecting the substrate muscle (see, for example, Gadelha, D. D., Filho, W. A., Brandão, M. A. J., and Montenegro, R. M. Jr. 2023. “Is Parathyroid Allotransplantation a Viable Option in the Treatment of Permanent Hypoparathyroidism? A Review of the Literature,” Endocrine (Totowa, New Jersey) 80(2):253-265; Rao, S. S., Rao, H., Moinuddin, Z., Rozario, A. P., and Augustine, T. 2023. “Preservation of Parathyroid Glands during Thyroid and Neck Surgery,” Online, Frontiers in Endocrinology (Lausanne, Switzerland) 14:1173950; Stevenson, A. and Mihai, R. 2018. “Patients' Views about Parathyroid Transplantation for Post-thyroidectomy Hypoparathyroidism,” Langenbeck's Archives of Surgery 403(5):623-629; Barkzynski, M., Golkowski, F., and Nawrot, I. 2017. “Parathyroid Transplantation in Thyroid Surgery,” Online, Gland Surgery (Hong Kong, China) 6(5):530-536; Lorente Poch, L., Sancho, J., Munoz, J. L., Gallego-Otaegui, L., Martines-Ruiz, C. and Sitges-Sierra, A. 2017. “Failure of Fragmented Parathyroid Gland Autotransplantation to Prevent Permanent Hypoparathyroidism after Total Thyroidectomy,” Langenbeck's Archives of Surgery 402(2):281-287; DeLeeuw. F., Breuskin, I., Abbaci, M., Casiraghi. O., Mirghani. H., and 3 others 2016. “Intraoperative Near-infrared Imaging for Parathyroid Gland Identification by Auto-fluorescence: A Feasibility Study,” World Journal of Surgery 40(9): 2131-2138; Lorente-Poch, L., Sancho, J. J., Ruiz, S., and Sitges-Serra, A. 2015. “Importance of in-Situ Preservation of Parathyroid Glands during Total Thyroidectormy,” British Journal of Surgery 102(4):359-367; Barkzynski, M., Cichon. S., Konturek, A., and Cichon, W. 2008. “Applicability of Intraoperative Parathyroid Hormone Assay during Total Thyroidectomy as a Guide for the Surgeon to Selective Parathyroid Tissue Autotransplantation,” World Journal of Surgery 32(5):822-828; Nawrot, I., Wozniewicz, B., Tolloczko, T., Sawiki, A., Gorski, A., and 7 others 2007. “Allotransplantation of Cultured Parathyroid Progenitor Cells without Immunosuppression: Clinical Results,” Transplantation 83(6):734-740; Lo, C. Y., Luk, J. M., and Tam, S. C. 2002. “Applicability of Intraoperative Parathyroid Hormone Assay during Thyroidectomy,” Annals of Surgery (Philadelphia, Pennsylvania) 236(5):564-569; Olson, J. A. Jr., DeBenedetti, M. K., Baumann, D. S., and Wells, S. A. Jr. 1996. “Parathyroid Autotransplantation during Thyroidectomy. Results of Long-term Follow-up,” Annals of Surgery (Philadelphia, Pennsylvania) 223(5):472-480).

Congenital hypoparathyroidism causes chronic hypocalcemia that leads to Nephrocalcinosis and renal failure. Its complications are numerous and severe, to include intractable heart failure and crippling neurological consequences. The tiny size of the parathyroids does not permit the use of vascular servovalves, hence, the implementation of the continuity of blood flow guideline otherwise demanded by the compound vascular bypass transplantation of organs and glands as intact. However, if harvested, the parathyroids would likewise remain intact, unminced, preferably in situ, normally towards the side edges on the front of the thyroid. Conventional parathyroid transplantation has sometimes been consistent with thyroid transplantation in mincing the parathyroids if not preparing these as a cell suspension for injection into muscle.

Fortuitously, however, “Parathyroid tissue can be transplanted without need for vascularization of the gland . . . ” (Garcia-Roca, R., Garcia-Aroz, S., Tzvetanov, I. Giulianotti, P. C., Campara, M., Oberholzer, H., and Benedetti, E. 2016. “Simultaneous Living Donor Kidney and Parathyroid Allotransplantation: First Case Report and Review of Literature,” Transplantation 100(6):1318-1321), and the parathyroids are uniquely manipulable to tolerate immunologically distinct tissue which other organs would promptly reject (Nawrot, I., Woniewicx, B., Tooczko, T., Sawicki, A., Grski. A., and 7 others 2007. “Allotransplantation of Cultured Parathyroid Progenitor Cells without Immunosuppression: Clinical Results,” Transplantation 83(6):734-740; Hasse. C., Klock, G., Schlosser, A. Zimmermann, U., and Rothmund, M. 1997. “Parathyroid Allotransplantation without Immunosuppression,” Lancet (London, England) 350(9087):1296-1297).

Thyroid transplantation must take into account the presence or absence of the normally four small parathyroids on the thyroid. If the parathyroids of both donor and recipient are unaffected and had provided normal calcium function, the donor parathyroids are kept with the donor for another patient in need of one or more normal parathyroids. If fewer are immediately evident on the graft gland, the graft should be closely examined to determine whether one or more parathyroids had anomalously become embedded within the parenchyma of the thyroid during development; if so, and the recipient has adequate native parathyroid function, the embedded parathyroid should be returned to the donor for another patient.

Where allograft had been necessitated by thyroid malignancy, dispersion of the pieces if rejected poses difficulty in later recovery (see, for example, Sakr, M., El-Kerm, Y., Abo-Elwafa, W., Mahmoud, A., and Fathi, I. 2018. “Heterotopic Thyroid Autotransplantation: A Preliminary Clinical Study,” Head and Neck 40(1):34-45; Mohsen, A. A., Nada, A. A., Ibrahim, M. Y., Ghaleb, A. H., Abou-Gabal, M. A., Mohsen, A. A., and Wassef, A. T. 2017. “Technique and Outcome of Autotransplanting Thyroid Tissue after Total Thyroidectomy for Simple Multinodular Goiters,” Asian Journal of Surgery 40(1):17-22; Sakr, M. and Mahmoud, A. 2017. “Review of Heterotopic Thyroid Autotransplantation,” Clinical and Experimental Otorhinolaryngology 10(4):289-295). Moreover, should the tissue become malignant, dispersion thus may increase the risk of metastasis. Autotransplantation in a solid block avoids this problem but does not eliminate the need for supplemental hormone therapy (Roy, P. G., Saund, M. S., Thusoo, T. K., Roy, D., and Sankar, R. 2003. “Fate of Human Thyroid Tissue Autotransplants,” Surgery Today 33(8):571-576).

Significantly, immunosuppressants no less a source of complications, preservation of the gland or glands intact allows the directly pipeline targeted delivery of anti-rejection drugs, thus materially reducing the odds for this factor as the cause of complications (see, for example, Barczyński, M., Golkowski, F., and Nawrot, I. 2017. “Parathyroid Transplantation in Thyroid Surgery,” Gland Surgery 6(5):530-536; Hicks, G., George, R., and Sywak, M. 2017. “Short and Long-term Impact of Parathyroid Autotransplantation on Parathyroid Function after Total Thyroidectomy,” Gland Surgery (Hong Kong, China) 6 (Supplement 1):S75-S85; Agha, A., Scherer, M. N., Moser, C., Karrasch, T., Girlich, C., and 4 others 2016. “Living-donor Parathyroid Allotransplantation for Therapy-Refractory Postsurgical Persistent Hypoparathyroidism in a Nontransplant Recipient-Three Year Results: A Case Report,” Online, BioMed Central Surgery (London, England) 16(1):51; Aysan, E., Altug, B., Ercan, C., Kesgin Toka, C., Idiz, U. O., and Muslumanoglu, M. 2016. “Parathyroid Allotransplant with a New Technique: A Prospective Clinical Trial,” Experimental and Clinical Transplantation 14(4):431-435; Khryshchanovich, V. and Ghoussein, Y. 2016. “Allotransplantation of Macroencapsulated Parathyroid Cells as a Treatment of Severe Postsurgical Hypoparathyroidism: Case Report,” Annals of Saudi Medicine 36(2):143-147; Ingulli, E. 2010. “Mechanism of Cellular Rejection in Transplantation,” Pediatric Nephrology (Berlin, Germany) 25(1):61-74; Tibell, A., Rafael, E., Wennberg, L., Nordenstrom, J., Bergstrom, M., and 6 others 2001. “Survival of Macroencapsulated Allogeneic Parathyroid Tissue One Year after Transplantation in Nonimmunosuppressed Humans,” Cell Transplantation 10(7):591-599; Tolloczko, T., Woźniewicz, B., Sawicki, A., and Gorski, A. 1997. “Allotransplantation of Cultured Human Parathyroid Cells: Present Ztatus and Perspectives,” Transplantation Proceedings 29(1-2):998-1000).

Whereas transplanted intact with blood supply and drainage, endocrine glands present a distinct outline, can be supported and treated with directly pipeline targeted drugs, clearly viewed with current imaging means, and if necessary, operated on or recovered laparoscopically with little difficulty, dispersed or distributed morcellated cells or fragments of endocrine glands or certain portions of glands cannot. Fragments can be differentially treated for contrast, but the glandular tissue disintegrated, are more difficult to retrieve. As opposed to unit integrity and clear demarcation from the surrounding tissue so that a graft organ can be squarely targeted, fragmentation makes revisiting or medicinally supporting a graft difficult, forcing systemic dispersal and side effects of the drug, making it inefficient and undependable.

At the same time, it has been known for at least 65 years that endocrine glands severed from connection to higher control by the hypothalamus remain functional (Wexler, B. C. 1980. “Transplantation of Pituitary and Adrenal glands of Spontaneously Hypertensive Rats into Hypophysectomized or Adrenalectomized, Normotensive Sprague-Dawley Rats,” British Journal of Experimental Pathology 61(4):429-439). Reasons recommending thyroid transplantation include autoimmune disease, accidental trauma, athyreosis, the agenesis or absence of a native thyroid gland or its dysfunctional condition, intractable thyroid deficiency, or hypothyroidism (hypothyrea, hypothyrosis, hypothyreosis hypothyroidea), surgical removal necessitated by carcinoma of sarcoma, and the inability of hormone supplementation to adapt to variations in the need for the level of secretion required in response to circadian and physiological conditions.

Analogous circumstances pertain to other glands such as the adrenals and parathyroids. The chronic problem of replacement gland availability is lessened by the switch method specified above in relation to heart transplantation, whereby the donor is placed on extracorporeal membrane oxygenation prior to death and the gland transplanted using the metered switch technique. This eliminates any interruption in perfusion and inherently accomplishes immune tolerance induction as the gland is gradually transferred from the circulatory system of the donor to that of the recipient using agents and over a period highly controllable, and which, if necessary, can extend over a period of days.

The metered switch technique blends the blood of the donor sustained on somatic support and the recipient, so that transplantation of a whole infant thymus past the normally prefibrosed period in development of the gland, eliminating the need to cut into the thymus and reducing the risk of graft versus host disease at the same time (Markert, M. L., Devlin, B. H., and Mccarthy, E. A. 2010. “Thymus Transplantation,” Clinical Immunology 135(2):236-246). The ability to use uninfected glands already fibrosed expands the zone of usable thymus glands to include those taken from older infants.

In orthotopic placement, transplantation is by using diversion servovalves to connect the internal thoracic arteries and veins of donor and recipient. The recipient with immature immune system and no thymus is then provided with a source of T cells to which the recipient was tolerance induced during transplantation (Puck, J. M. 2019. “Newborn Screening for Severe Combined Immunodeficiency and T-cell Lymphopenia,” Immunological Reviews 287(1):241-252). Treatment of the innervation varies according to whether the recipient is completely athymic or born with a gland anomalous in this regard.

The prosthetic disorder response system implanted in the recipient both administers the transfer, and once transplanted, remains in place in the recipient to directly pipeline target supportive medication such as immunosuppressants, anti-inflammatoires, and antimicrobials to the blood supply of the transplant. Switch transplantation of a complete gland is by the same method as described for the heart. If orthotopic then the same vessels of donor and recipient are used; if heterotopic, suitable vessels at the new site are used.

Where missing or defective function is exclusively adrenomedullary or adrenocortical, an autologous transplant consisting of an entire or only the affected portion of the gland can be transplanted. If the latter, the portion is transplanted not using the switch method but inserted where to spot the graft with imaging with magnetic resonance imaging, computed tomography, or photon-emission tomography, for example, will pose little difficulty without contrast. Placement can be between the renal capsule and into the cortex sufficiently that supply and drainage vessels and a fibrous capsule develop.

Continued improvements in imaging, surgical technique, and immunosuppression have allowed improved outcomes in the homotransplantation of glands (Dubernard, J. M., Cloix, P., Tajra, L. C., Alduglihan, W., Borson, F., Lefrancois, N., and Martin, X. 1995. “Simultaneous Adrenal Gland and Kidney Allotransplantation after Synchronous Bilateral Renal Cell Carcinoma: A Case Report,” Transplantation Proceedings 27(1):1320-1321; Patino, J. F. and Fenn, J. E. 1993. “A Successful Transplant of Embryonic Adrenal [cortical] Tissue in a Patient with Addison's Disease,” Yale Journal of Biology and Medicine 6(1): 3-10; Yu. X. C., Yu T. L., Zhang. S. Z., Liu D. F., and Jia, J. H. 1991. “Homotransplantation of Adrenal Gland,” Chinese Medical Journal (English edition) 104(6):487-490; Katz, F. and Mainzer, F. 1941. “Successful Grafting of Adrenal Gland in a Case of Addison's Disease,” Online, British Medical Journal 1(4190):617-618; Beer. E. and Oppenheimer, B. S. 1934. “Transplantation of the Adrenal Cortex for Addison's Disease,” Annals of Surgery (Philadelphia, Pennsylvania) 100(4):689-703).

While in the ability to spontaneously adapt to the activity level and physiological status of the recipient as to provide a more physiological result, morcellation (morcellement) and implantation into skeletal muscle represent a fundamental improvement in treatment over hormone supplementation (Grodstein, E., Hardy, M. A., and Goldstein, M. J. 2010. “A Case of Human Intramuscular Adrenal Gland Transplantation as a Cure for Chronic Adrenal Insufficiency,” American Journal of Transplantation 10(2):431-433), the superficial positioning of an allogeneic gland in morcellated form into pockets in skeletal muscle makes difficult the locating and thorough recovery of all the cells in the event of a late adverse sequela, affords little protection against accidental injury, and confounds the ability to differentially access the graft for any purpose, forcing the use of untargeted or indiscriminate treatment. In that albeit different, the need for continued treatment continues, characterization of an organ transplant as a cure is deceptive; a cure would mean that the adverse condition had been completely eliminated.

A general principle pertinent to allogeneic graft materials whether whole organs or processed portions thereof is that in the event of intractable rejection, infection, or any other irreversible failure, a means for subsequent recoverability or elimination which is prompt and minimally if at all injurious to the patient is imperative. More generally, anything placed in the patient must be readily accessible to diagnosis and therapy, and preferably as distinct from other tissue—that is, targeted.

While positioned deeper within the body, should the need arise to recover alginate-encapsulated cells injected intraperitoneally under the kidney capsule (Balyura, M., Gelfgat. E., Ehrhart-Bomstein, M., Ludwig. B., Gendler, Z., and 6 others 2015. “Transplantation of Bovine Adrenocortical Cells Encapsulated in Alginate,” Online, Proceedings of the National Academy of Sciences of the United States of America 112(8):2527-2532) likewise poses difficulty of selective accessibility for any purpose, to include thorough recovery of destruction, whether by excision, aspiration, or the application of heat. No such enzyme as alginase exists in mammals, but “ionically cross-linked alginate gels can be dissolved by release of the divalent ions cross-linking the gel into the surrounding media due to exchange reactions with monovalent cations such as sodium ions” (Lee, K. Y. and Mooney, D. J. 2012. “Alginate: Properties and Biomedical Applications,” Progress in Polymer Science 37(1):106-126).

If allogeneic, the result of a breach in the outer capsule or envelope would likely be an acute immune reaction, and if xenogeneic (Balyura, M., Gelfgat, E., Ehrhart-Bomstein, M., Ludwig, B., Gendler, Z., and 6 others 2015, Op cit. in this section), a hyperacute reaction.

Even if intact, alginate encapsulation systems tend not to be stable for more than one week in vivo, and even when dual-crosslinked, alginate microbeads if challenged with an inflammatory reaction by injection in a lipopolysaccharide, remain stable for no more than 3 weeks (Somo, S. I. Langert, K., Yang C. Y., Vaicik, M. K. Ibarra, V., and 4 others 2018. “Synthesis and Evaluation of Dual Crosslinked Alginate microbeads,” Acta Biomaterialia 65:53-65), and if monolayered or thinner, an alginate capsule or envelope about a cell challenged thus would break down in less time.

Not intentionally challenged thus, the mechanical stability of alginate-encapsulated cells or microbeads as applied to the treatment of diabetes would hold up for a longer time before initiating an immune reaction. To date, alginate is adequate for the short-term time-release of drugs, but not for encapsulating allogeneic or xenogeneic contents for the life of the patient. Transplantation of an intact autologous gland or glands so that immune tolerance induction continues through shared circulation through the graft during transfer from the donor to the recipient and so that drugs such as immunosuppressants can be directly pipeline targeted through one or more accessory channels in a vascular servovalve jacket about the gland blood supply is plainly more durable.

Add to this the fact that a glandular graft is no less susceptible to ischemia-reperfusion injury than a solid organ, and the totality of factors establish metered switch technique as superior to any alternative method of transplantation. This is so whether the alternative form of transplantation consists of insertion of the allograft (homograft) as intact, in cubes or cells, even when alginate-encapsulated for insertion into a skeletal muscle such as the sternocleidomastoideoectoralis major, or rectus abdominus, or subperitoneally, or beneath the renal capsule. For the foreseeable future, it is also superior to the implantation of a synthetic thyroid gland produced by means of three dimensional bioprinting.

Given conventional methods for ameliorating such conditions, it is essential to establish the superiority of heterotopic transplantation of an intact thyroid allograft over alternative approaches, all of which entail a period of interrupted perfusion and thus, a critical degradation in the graft organ endothelium, promoting endothelial vasculitis and injury upon reperfusion. While pertinent at a lower level, inserting diced thyroid into muscle still involves an interval of anoxia. A cancerous thyroid which must be completely removed can be replaced with a healthy thyroid from a ‘brain-dead’ donor using the sudden or metered switch technique.

Successful robotic total thyroidectomy and heterotopic autotransplantation in muscle or orthotopic replacement of a primary carcinomatous thyroid gland with one intact taken from a deceased donor on life support is beneficial by reducing, perhaps eventually replacing the need for endocrinological support, usually consisting of levothyroxine replacement therapy on a lifelong basis, with dependency upon compliance and leading to several side effects, to include anxiety, insomnia, tachycardia, tremor, sweating, hyperhidrosis, and heat sensitivity. Absent the application of switch transplantation, all sources of a complete replacement thyroid or the equivalent, to include diced allograft or autograft infixed within muscle, conventional allograft, or bioprinted thyroid are subjected to a period of anoxia, degrading the graft and increasing the odds of adverse sequelae.

Using switch transplantation, once removal of the diseased thyroid gland by means of a more recent scarless procedure demanding experience and skill as to prompt remote robotic accomplishment (see, for example, Tae, K., Ji. Y. B., Song, C. M., and Ryu, J. 2019. “Robotic and Endoscopic Thyroid Surgery: Evolution and Advances,” Clinical and Experimental Otorhinolaryngology 12(1):1-11: Camenzuli. C., Schembri Wismayer, P., and Calleja Agius, J. 2018. “Transoral Endoscopic Thyroidectomy: A Systematic Review of the Practice So Far,” Journal of the Society of Laparoendoscopic Surgeons 22(3). pii: e2018.00026; Dionigi, G., Chai, Y. J., Tufano, R. P., Anuwong, A., and Kim, H. Y. 2018. “Transoral Endoscopic Thyroidectomy via a Vestibular Approach: Why and How?,” Endocrine 59(2):275-279; Razavi, C. R., Khadem, M. G. A., Fondong, A., Clark, J. H., Richmon, J. D., Tufano, R. P., and Russell, J. O. 2018. “Early Outcomes in Transoral Vestibular Thyroidectomy: Robotic versus Endoscopic Techniques,” Head and Neck 40(10):2246-2253; Müller, V., Mogl. M., Seika, P., Jons. T., Sauer, I, and 3 others 2018. “How I Do It: New Dissector Device Allows for Effective Operative Field in Transoral Endoscopic Thyroid Surgery Using Vestibular Approach,” Surgical Innovation 25(5):444-449; Zorron. R., Bures, C., Brandl, A., Seika, P., and Müller, V. 2018. “Tips and Technical Issues for Performing Transoral Endoscopic Thyroidectomy with Vestibular Approach (TOETVA): A Novel Scarless Technique for Neck Surgery,” (in German with English abstract at Pubmed), Chirurg 89(7):529-536; Le, Q. V., Ngo. D. Q., and Ngo, Q. X. 2018. “Transoral Endoscopic Thyroidectomy Vestibular Approach (TOETVA): A Case Report as New Technique in Thyroid Surgery in Vietnam,” International Journal of Surgery Case Reports 50: 60-63; Anuwong, A., Sasanakietkul. T., Jitpratoom, P., Ketwong, K., Kim, H. Y., Dionigi, G., and Richmon, J. D. 2018. “Transoral Endoscopic Thyroidectomy Vestibular Approach (TOETVA): Indications, Techniques and Results,” Surgical Endoscopy 2018 32(1):456-465: Russell, J. O., Razavi, C. R., Al Khadem, M. G., Lopez, M., Saraf. S., and 4 others 2018. “Anterior Cervical Incision-sparing Thyroidectomy: Comparing Retroauricular and Transoral approaches,” Laryngoscope Investigative Otolaryngology 3(5):409-414; Russell, J. O., Razavi, C. R., Garstka, M. E., Chen, L. W., Vasiliou, E., Kang. S. W., Tufano, R. P., and Kandil, E. 2018. “Remote-access Thyroidectomy: A Multi-institutional North American Experience with Transaxillary, Robotic Facelift, and Transoral Endoscopic Vestibular Approaches,” Journal of the American College of Surgeons 228(4):516-522; Shan, L. and Liu, J. 2018. “A Systemic Review of Transoral Thyroidectomy,” Surgical Laparoscopy, Endoscopy, and Percutaneous Techniques 28(3):135-138; Chen, S., Zhao, M., and Qiu, J. 2018. “Transoral Vestibule Approach for Thyroid Disease: A Systematic Review,” European Archives of Otorhinolaryngology November 20; Jimenez Lillo, J and Martin-Perez, B. 2018. “5 Things to Know about Transoral Endoscopic Thyroidectomy Vestibular Approach (TOETVA),” Advances in Surgery, available Online at https://aischannel.com/society/5-things-know-transoral-endoscopic- thyroidectomy-vestibular-approach-toetva/; Anuwong, A., Kim, H. Y., and Dionigi, G. 2017. “Transoral Endoscopic Thyroidectomy Using Vestibular Approach: Updates and Evidences,” Gland Surgery 6(3):277-284; Joliat, G. R., Guarnero, V., Demartines, N., Schweizer. V., and Matter, M. 2017. “Recurrent Laryngeal Nerve Injury after Thyroid and Parathyroid Surgery: Incidence and Postoperative Evolution Assessment,” Medicine (Baltimore, Maryland) 96(17):e6674; Chang, E. H. E., Kim, H. Y., Koh, Y. W., and Chung. W. Y. 2017. “Overview of Robotic Thyroidectomy,” Gland Surgery 6(3):218-228; Akkari, M., Makeieff. M., Jeandel. C., Raingeard. I., Cartier, C., and 4 others 2014. “Thyroid Surgery in Children and Adolescents: A Series of 65 Cases,” European Annals of Otorhinolaryngology, Head and Neck Diseases 131(5):293-297; Byeon, H. K., Holsinger, F. C., Tufano, R. P., Chung, H. J., Kim, W. S., Koh. Y. W., and Choi. E. C. 2014. “Robotic Total Thyroidectomy with Modified Radical Neck Dissection via Unilateral Retroauricular Approach,” Annals of Surgical Oncology 21(12):3872-3875) has been completed, the balance of the operation is relatively uncomplicated, and the thyroid allograft can be directly pipeline-targeted drugs to include immunosuppressants and a steroid automatically from implanted drug reservoirs, thus avoiding the risk of immunosuppressant-induced cancer recurrence.

The thyroid graft is dissected out of the deceased donor having remained on life support with the suspensory or Berry's ligament of the gland, and positioning the replacement thyroid in a less crowded site with more direct access to a blood supply and drainage, depending upon the individual anatomy, along the abdominal aorta, esophagus, or somewhat farther down the trachea (see, for example, Allen, E. and Fingeret, A. 2019. “Anatomy, Head and Neck, Thyroid” Online, Treasure Island, Florida, at https://www.nebi.nlm.nih.gov/books/NBK470452/,” Wijerathne, S., Goh, X., and Parameswaran, R. 2019. “Ipsilateral Nonrecurrent Laryngeal Nerve Palsy and Delayed Palsy of the Contralateral Recurrent Laryngeal Nerve in a Case of Third-time Reoperative Thyroid Surgery,” Annals of the Royal College of Surgeons of England 101(2):e55-e58; Butskiy, O., Chang, B. A., Luu, K., Mckenzie. R. M., and Anderson, D. W. 2018. “A Systematic Approach to the Recurrent Laryngeal Nerve Dissection at the Cricothyroid Junction,” Journal of Otolaryngology—Head and Neck Surgery 47(1):57; Sun, H., Zanghi, G. N., Freni. F., and Dionigi, G. 2018. “Continuous and Intermitted Nerve Monitoring in Thyroid Surgery: Two Complementary Devices,” Gland Surgery 7 (Supplement 1):S80-S81; Hales, N. W., Kamani, D., and Randolph, G. W. 2018. “Recurrent Laryngeal Nerve Preservation in Thyroid Cancer Involving the Ligament of Berry,” Operative Techniques in Otolaryngology 29(1):14-18; Setiawan, I. G. B. and Adiputra, P. A. T. 2018. A Successful Tracheal Resection and Anastomosis in Papillary Thyroid Carcinoma with Tracheal Invasion,” Open Access Macedonian Journal of Medical Sciences 6(11):2161-2164; Kang. K. H., Song. R. Y., Suh, Y. J., and Park, S. J. 2018. “Safety of Medial Dissection of the Thyroid Gland along the Trachea Based on Anatomic Constancy of the Laryngeal Entry Point of the Recurrent Laryngeal Nerve,” Annals of Surgical Treatment and Research 95(1):16-21; Naraynsingh, V., Cawich, S., Hassranah, D., Maharaj. R., Islam, S., and Singh, Y. 2018. “Retrograde Thyroidectomy for Preservation of the External Branch of the Superior Laryngeal Nerve: A Case Series,” International Journal of Surgery Case Reports 53:517-521; Demiryas, S., Donmez, T., and Cekic, E. 2018. “Effect of Nerve Monitoring on Complications of Thyroid Surgery,” Northern Clinics of Istanbul 5(1):14-19; Tanal. M. and Uludag, M. 2018. “The Effects of Neuromonitorization in Thyroidectomies Can Be Safely Evaluated with the Standardized Technique,” Northern Clinics of Istanbul 5(3):274-275; Schneider, R., Machens, A., Randolph, G. W., Kamani, D., Lorenz, K., and Dralle, H. 2017. “Opportunities and Challenges of Intermittent and Continuous Intraoperative Neural Monitoring in Thyroid Surgery,” Gland Surgery 6(5):537-545; Rajabian, A., Walsh, M., and Quraishi, N. A. 2017. “Berry's Ligament and the Inferior Thyroid Artery as Reliable Anatomical Landmarks for the Recurrent Laryngeal Nerve (RLN): A Fresh-cadaveric Study of the Cervical Spine. The RLN Relevant to Spine,” Spine Journal 17(3S): S33-S39; Wojtczak, B., Sutkowski, K., Kaliszewski, K., Głod, M., and Barczyński, M. 2017. “Experience with Intraoperative Neuromonitoring of the Recurrent Laryngeal Nerve Improves Surgical Skills and Outcomes of Non-monitored Thyroidectomy,” Langenbeck's Archives of Surgery 402(4):709-717; Wu, C. W., Hao, M., Tian, M., Dionigi, G., Tufano, R. P., and 7 others 2017. “Recurrent Laryngeal Nerve Injury with Incomplete Loss of Electromyography Signal during Monitored Thyroidectomy-evaluation and Outcome,” Langenbeck's Archives of Surgery 402(4):691-699; Le, Q. V., Ngo, D. Q., and Ngo, Q. X. 2018. “Non-recurrent Laryngeal Nerve in Thyroid Surgery: A Report of Case Series in Vietnam and Literature Review,” International Journal of Surgery Case Reports 50:56-59; Henry, B. M., Sanna, B., Graves, M. J., Sanna, S., Vikse, J., and 3 others 2017. “The Reliability of the Tracheoesophageal Groove and the Ligament of Berry as Landmarks for Identifying the Recurrent Laryngeal Nerve: A Cadaveric Study and Meta-analysis,” Online, Biomed Research International 2017:4357591; Christoforides, C., Papandrikos, I., Polyzois, G., Roukounakis, N., Dionigi, G., and Vamvakidis, K. 2017. “Two-stage Thyroidectomy in the Era of Intraoperative Neuromonitoring,” Gland Surgery 6(5):453-463; Henry, B. M., Sanna, B., Vikse, J., Graves, M. J., Spulber, A., and 4 others 2017. “Zuckerkandl's Tubercle and Its Relationship to the Recurrent Laryngeal Nerve: A Cadaveric Dissection and Meta-analysis,” Auris, Nasus, Larynx 44(6):639-647; Kandil, E., Anwar, M. A., Bamford, J., Aslam, R., and Randolph, G. W. 2017. “Electrophysiological Identification of Nonrecurrent Laryngeal Nerves,” Laryngoscope 127(9):2189-2193; Henry, B. M., Pekala, P. A., Sanna, B., Vikse, J., Sanna, S., and 4 others 2017. “The Anastomoses of the Recurrent Laryngeal Nerve in the Larynx: A Meta-Analysis and Systematic Review,” Journal of Voice 31(4):495-503; Rotolo, N., Cattoni. M., and Imperatori, A. 2017. “Complications from Tracheal Resection for Thyroid Carcinoma,” Gland Surgery 6(5):574-578; Wu. G. and Wang. K. 2017. “A Novel Variation of the Recurrent Laryngeal Nerve,” Online, BioMed Central Surgery 17(1):66; Uludag, M., Aygun, N., Kartal, K., Citgez, B., Besler, E., and 5 others 2017. “Contribution of Intraoperative Neural Monitoring to Preservation of the External Branch of the Superior Laryngeal Nerve: A Randomized Prospective Clinical Trial,” Langenbeck's Archives of Surgery 402(6):965-976; Schneider, R., Randolph, G. W., Barczynski, M., Dionigi, G., Wu. C. W., and 4 others 2016. “Continuous Intraoperative Neural Monitoring of the Recurrent Nerves in Thyroid Surgery: A Quantum Leap in Technology,” Gland Surgery 5(6):607-616; Simon, D., Boucher, M., and Schmidt-Wilcke, P. 2015. “Intraoperative Avoidance and Recognition of Recurrent Laryngeal Nerve Palsy in Thyroid Surgery,” (in German with English abstract at Pubmed), Chirurg 86(1): 6-12; Schneider. R., Sekulla, C., Machens. A., Lorenz, K., Nguyen Thanh, P., and Dralle, H. 2015. “Postoperative Vocal Fold Palsy in Patients Undergoing Thyroid Surgery with Continuous or Intermittent Nerve Monitoring,” British Journal of Surgery 102(11):1380-1387; Lin, S., Huang, H., Liu, X., Li, Q., Yang, A., Zhang, Q., Guo, Z., and Chen, Y. 2014. “Treatments for Complications of Tracheal Sleeve Resection for Papillary Thyroid Carcinoma with Tracheal Invasion,” European Journal of Surgical Oncology 40(2):176-181; Leow, C. K. and Webb, A. J. 1998. “The Lateral Thyroid Ligament of Berry,” International Surgery 83(1):75-78; Sasou, S., Nakamura, S., and Kurihara, H. 1998. “Suspensory Ligament of Berry: Its Relationship to Recurrent Laryngeal Nerve and Anatomic Examination of 24 Autopsies,” Head and Neck 20(8):695-698).

The suspension structure of the ligaments not lending itself to transplantation, positioning of the transplant thyroid at a lower level along the trachea, esophagus, or abdominal aorta, for example, is not with the Berry's ligaments of either the native or transplant glands. Unless implicated in metastasis-less likely if radioiodine is administered to destroy any remnant of the diseased gland—the native ligaments are cut with the attached or tracheal ends left in the original positions, reducing the risk of injury to the recurrent laryngeal nerve. Fixation along the lower trachea, peristaltic esophagus, or pulsatile abdominal aorta is through the deliberate provocation of cohesive adhesions to take the place of native suspension ligaments.

More specifically, the replacement thyroid is stitched to the substrate structure with absorbable suture, the interface between the gland and substrate deliberately scored, or ‘roughed up.’ or chemically treated (poudrage) to encourage adhesion, a process routinely applied in pleurodesis (see, for example, Eldridge. L. 2018. “Pleurodesis Indications, Procedure, and Complications,” Verywell Health, available Online at https://www.verywellhealth.com/what-type-of-procedure-is-a-pleurodesis-2249164) to bind the visceral to the parietal pleura and thus obliterate the pleural space and prevent pleural effusion (see, for example, Lv, L., Zhou, M., Pan, S., and Yang, K. 2015. “Reduced Incidence of Postoperative Symptoms following a Novel Bilateral Supraclavicular Approach to Open Thyroidectomy: A Randomized Clinical Trial in a Chinese Population,” International Journal of Clinical and Experimental Medicine 8(5):7359-7366; Editorial Comment, Canadian Medical Association Journal 1935. “Pleural Poudrage,” Canadian Medical Association Journal, 33(3):312; Bethune, N. 1935. “Pleural Poudrage: New Technique for the Deliberate Production of Pleural Adhesion as Preliminary to Lobectomy,” Journal of Thoracic Surgery 4:251).

Areas surrounding or opposing those scored or chemically treated to cohere can be treated with any of numerous substances such as autologous blood, bleomycin, tetracycline, doxycycline, iodine, povidone-iodine, or tale slurry not containing asbestiform fibres (see below) whether thoracoscopic or insufflated to prevent adhesions from extending into areas that should remain separate (see, for example, Torii. H., Takagi. T., Urabe, M., Tsujimoto, H., Ozamoto, Y., and 3 others 2017. “Anti-adhesive Effects of a Newly Developed Two-layered Gelatin Sheet in Dogs,” Journal of Obstetrics and Gynaecology Research 43(8):1317-1325; Makay, O., Isik, D., Erol. V., Yenisey, C., Kose. T., and 4 others 2017. “Efficacy of Simvastatin in Reducing Postoperative Adhesions after Thyroidectomy: An Experimental Study,” Acta Chirurgica Belgica 117(2):77-83; Naito, M., Ogura, N., Yamanashi, T., Sato, T., Nakamura. T., and 6 others 2017. “Prospective Randomized Controlled Study on the Validity and Safety of an Absorbable Adhesion Barrier (Interceed®) Made of Oxidized Regenerated Cellulose for Laparoscopic Colorectal Surgery,” Asian Journal of Endoscopic Surgery 10(1):7-11; Luo. L., Zhang, C., Zhao, J., Wei, Q., and Li, X. 2015. “Effects of Rapamycin on Reduction of Peridural Fibrosis: An Experimental Study,” Medical Science Monitor 21:482-488; Hu, Y., Yamashita, K., Tabayashi, N., Abe. T., Hayata, Y., and 6 others 2015. “Gelatin Sealing Sheet for Arterial Hemostasis and Anti-adhesion in Vascular Surgery: A Dog Model Study,” Biomedical Materials and Engineering 25(2):157-168; Idiz, O., Aysan, E., Firat, D., Ersoy, Y. E., Cengiz, M. B., and 3 others 2014. “Efficacy of Glycerol and Flax Seed Oil as Anti-adhesive Barriers after Thyroidectomy,” Medical Science Monitor 20:1090-1094; Tsujimoto, H., Takamori, H., Tsuji, M., Hayashi, M., Ikeda, J., and 7 others 2014. “Development of Gelatin Flakes, a New Type of Anti-adhesive Material: A Preliminary Study of in Vivo Rat Adhesion Models,” Surgery Today 44(2):391-394; Park, K. S., Lee, K. E., Ku do, H., Kim, S., Park, W. S., and 3 others 2013. “Antiadhesive Effect and Safety of Oxidized Regenerated Cellulose after Thyroidectomy: A Prospective, Randomized Controlled Study,” Journal of the Korean Surgical Society 84(6):321-329; Bae, D. S., Woo, J. W., Paek, S. H., Kwon, H., Chai, Y., and 4 others 2013. “Antiadhesive Effect and Safety of Sodium Hyaluronate-carboxymethyl Cellulose Membrane in Thyroid Surgery,” Journal of the Korean Surgical Society 85(5):199-204).

While one of the most effective substances available for pleurodesis, concern for the use of tale not containing asbestos fibers is readily avoided by using any of numerous alternative substances for the purpose, several specified above, especially povidone-iodine or autologous blood, which carry no such risk and are low in cost. Neither does surgical pleurodesis proffer such risk, whether performed through a thoracotomy or video-assisted thoracoscopic surgery. The literature betrays a lack of unanimity as to whether tale not containing asbestos fibers is still a carcinogen.

Unlike the long-term daily occupational exposure of workers in the tale industry, the tale used in pleurodesis over the small posterior surface of the thyroid gland is not inhaled over the relatively brief interval of the procedure by the surgical staff wearing masks, nor is the patient exposed to airborne talc (see, for example, Keeratichananont, W., Kaewdech, A., and Keeratichananont, S. 2018. “Efficacy and Safety Profile of Autologous Blood versus Tale Pleurodesis for Malignant Pleural Effusion: A Randomized Controlled Trial,” Therapeutic Advances in Respiratory Disease 12:1753466618816625; Koegelenberg, C. F. N., Shaw, J. A., Irusen, E. M., and Lee, Y. C. G. 2018. “Contemporary Best Practice in the Management of Malignant Pleural Effusion,” Therapeutic Advances in Respiratory Disease 12:1753466618785098; Terra, R. M. and Dela Vega, A. J. M. 2018. “Treatment of Malignant Pleural Effusion,” Journal of Visualized Surgery 4:110; Fitzgerald, D. B., Koegelenberg, C. F. N., Yasufuku, K., and Lee, Y. C. G. 2018. “Surgical and Non-surgical Management of Malignant Pleural Effusions,” Expert Review of Respiratory Medicine 12(1):15-26; Chang, C. J., Tu. Y. K., Chen, P. C., and Yang, H. Y. 2017. “Occupational Exposure to Tale Increases the Risk of Lung Cancer: A Meta-analysis of Occupational Cohort Studies,” Canadian Respiratory Journal 2017:1270608; Xia, H., Wang, X. J., Zhou, Q., Shi, H. Z., and Tong, Z. H. 2014. “Efficacy and Safety of Tale Pleurodesis for Malignant Pleural Effusion: A Meta-analysis,” Online, PLoS [Public Library of Science] One 9(1):e87060; Wild, P. 2006. “Lung Cancer Risk and Tale Not Containing Asbestiform Fibres: A Review of the Epidemiological Evidence,” Occupational and Environmental Medicine 63(1):4-9; Aelony, Y. and Yao, J. F. 2005. “Prolonged Survival after Tale Poudrage for Malignant Pleural Mesothelioma: Case Series,” Respirology (Carlton, Victoria, Australia) 10(5):649-655).

Attachment in the recipient The valve-jackets used are solenoid-driven, switched by the operator using a common switch to assure definitive placement of the graft organ in either the circulatory system of the donor or the recipient. Three dimensional bioprinting of a human thyroid gland remains in an early stage of development, and will probably take at least a decade before approval by the U.S. Food and Drug Administration for implantation of a thyroid gland in a human recipient. To develop normally, the prospective endothelium of a bioprinted thyroid will have to develop to normally respire, pass nutrients, and release waste. To normally develop, the vascular tissue will therefore have to be continuously washed over by blood throughout production. Implantation without ischemia, that is, with continuous perfusion to avoid reperfusion injury with endothelial damage and vasculitis thus pertains equally to a bioprinted or an allograft thyroid.

A central advantage in sudden and metered compound vascular bypass, or sudden switched-organ transplantation is that it eliminates ischemia. To date, such successes in allograft function as have been reported are confined to the short term in rodent models. The biofabrication of functional vasculature deep within an artificial organ or gland is more challenging than the making of artificial vessels. Essentially, once assimilated, a native allograft eliminates the need for lifelong medication, and the successful production of an artificial human thyroid is not likely for a dozen or more years.

All methods for implantation other than the switch techniques involve an unnatural disconnection from a continuous blood supply and a period of ischemia. The sudden and metered compound vascular bypass, or sudden switched-organ transplantation techniques will therefore apply no less to the implantation of artificial, that is, bioengineered or bioprinted organs, glands, and tissues once developed as to those completely biological.

Currently, the biofabrication of vascular tissue able to simulate normal physiology is being pursued but has not been achieved (see, for example, Assad, H., Assad, A., and Kumar, A. 2023. “Recent Developments in 3D Bio-printing and Its Biomedical Applications,” Online, MDPI [Multidisciplinary Digital Publishing Institute] Pharmaceuties (Basel, Switzerland) 15(1):255; Jiang. W., and Mei. H., Zhao, S. 2021. “Applications of 3D Bio-printing in Tissue Engineering and Biomedicine,” Journal of Biomedical Nanotechnology (Stevenson Ranch, California) 17(6):989-1006; Jafarkhani, M., Salehi, Z., Aidun, A., and Shokrgozar, M. A. 2019. “Bioprinting in Vascularization Strategies,” Online, (Tehran, Irran) Iranian Biomedical Journal 23(1):9-20; Pi, Q., Maharjan, S., Yan, X., Liu, X., Singh, B.8., and 12 others 2018. “Digitally Tunable Microfluidic Bioprinting of Multilayered Cannular Tissues,” Advanced Materials (Deerfield Beach, Florida) 30(43):e1706913; Miri, A. K., Khalilpour, A., Cecen, B., Maharjan, S., Shin, S. R., and Khademhosseini, A. 2018. “Multiscale Bioprinting of Vascularized Models,” Biomaterials pii: S0142-9612(18) 30560-X; Kačarević, Ž. P., Rider, P. M., Alkildani, S., Retnasingh, S., Smeets. R., and 3 others 2018. “An Introduction to 3D Bioprinting: Possibilities, Challenges and Future Aspects,” Materials (Basel, Switzerland) 11(11). pii: E2199; Xia, Z., Kang. B., Shin, J., Park, H. J., Rhyou, C., Kang. D., and 4 others 2018. “High-resolution Acoustophoretic 3D Cell Patterning to Construct Functional Collateral Cylindroids for Ischemia Therapy,” Nature Communications 9(1):5402; Xia, Z., Jin, S., and Ye. K. 2018. “Tissue and Organ 3D Bioprinting,” Society for Laboratory Automation and Screening Technology (Thousand Oaks, California) 23(4):301-314; Anwar, S., Singh, G. K., Miller, J., Sharma, M., Manning, P., and 3 others 2018. “3D Printing is a Transformative Technology in Congenital Heart Disease,” Online, Journal of the American College of Cardiology. Basic to Translational Science (New York, New York) 3(2):294-312; Sasmal, P., Datta. P., Wu, Y., and Ozbolat, I. T. 2018. “3D Bioprinting for Modelling Vasculature,” Online, Microphysiological Systems November 2:9, Online at http://mps.amegroups.com/article/view/4811/5584; Xu, Y., Hu. Y., Liu, C., Yao. H., Liu, B., and Mi. S. 2018. “A Novel Strategy for Creating Tissue-engineered Biomimetic Blood Vessels Using 3D Bioprinting Technology,” Materials (Basel, Switzerland) 11(9). pii: E1581; Bulanova, E. A., Koudan, E. V., Degosserie, J., Heymans. C., Pereira, F. D., and 10 others 2017. “Bioprinting of a Functional Vascularized Mouse Thyroid Gland Construct,” Biofabrication 9(3):034105; Datta, P., Ayan, B., and Ozbolat. I. T. 2017. “Bioprinting for Vascular and Vascularized Tissue Biofabrication,” Acta Biomaterialia 51:1-20; Duan, B. 2017. “State-of-the-art Review of 3D Bioprinting for Cardiovascular Tissue Engineering,” Annals of Biomedical Engineering 45(1):195-209; Leberfinger, A. N., Ravnic, D. J., Dhawan, A., and Ozbolat. I. T. 2017. “Concise Review: Bioprinting of Stem Cells for Transplantable Tissue Fabrication,” Online, Stem Cells Translational Medicine (Oxford, England) 6(10): 1940-1948; Richards, D., Jia. J., Yost, M., Markwald, R., and Mei, Y. 2017. “3D Bioprinting for Vascularized Tissue Fabrication,” Annals of Biomedical Engineering (New York, New York) 45(1):132-147; Jang. J. 2017. “3D Bioprinting and In Vitro Cardiovascular Tissue Modeling,” Online, MDPI [Multidisciplinary Digital Publishing Institute] Bioengineering (Basel, Switzerland) 4(3). pii: E71; Massa, S., Sakr, M. A., Seo. J., Bandaru, P., Arneri, A., and 13 others 2017. “Bioprinted 3D Vascularized Tissue Model for Drug Toxicity Analysis,” Biomicrofluidics 11(4):044109; Zhang, Y. S., Pi. Q., and van Genderen, A. M. 2017. “Microfluidic Bioprinting for Engineering Vascularized Tissues and Organoids,” Journal of Visualized Experiments(126); Jia, W., Gungor-Ozkerim, P. S., Zhang, Y. S., Yue, K., Zhu, K., and 6 others 2016. “Direct 3D Bioprinting of Perfusable Vascular Constructs Using a Blend Bioink,” Biomaterials (Amsterdam, North Holland, Netherlands) 106:58-68; Patra, S. and Young. V. 2016. “A Review of 3D Printing Techniques and the Future in Biofabrication of Bioprinted Tissue,” Cell Biochemistry and Biophysics 74(2):93-98; Ji, S. and Guvendiren, M. 2017. “Recent Advances in Bioink Design for 3D Bioprinting of Tissues and Organs,” Online, Frontiers in Bioengineering and Biotechnology (Lausanne, Switzerland) 5:23; Mandrycky, C., Wang, Z., Kim, K., and Kim, D. H. 2016. “3D Bioprinting for Engineering Complex Tissues,” Biotechnology Advances 34(4):422-434; Paulsen, S. J. and Miller, J. S. 2015. “Tissue Vascularization through 3D Printing: Will Technology Bring Us Flow?,” Developmental Dynamics 244(5):629-640; Kuo, K. C., Lin, R. Z., Tien, H. W., Wu, P. Y., Li, Y. C., Melero-Martin, J. M., and Chen, Y. C. 2015. “Bioengineering Vascularized Tissue Constructs Using an Injectable Cell-laden Enzymatically Crosslinked Collagen Hydrogel Derived from Dermal Extracellular Matrix,” Acta Biomaterialia 27:151-166; Skardal, A., Devarasetty, M., Kang, H. W., Mead, I., Bishop, C., and 6 others 2015. “A Hydrogel Bioink Toolkit for Mimicking Native Tissue Biochemical and Mechanical Properties in Bioprinted Tissue Constructs,” Acta Biomaterialia 25:24-34).

Congenital hypothyroidism can denote an underactive thyroid gland as in ordinary hypothyroidism (hypothyrea, hypothyrosis, hypothyreosis hypothyroidea) or the congenital absence of a thyroid gland as an anatomical anomaly (athyroidism, thyroid agenesis, thyroid aplasia). Delivery through the umbilical cord wanting, and untreated by supplementation of thyroxine T4 throughout gestation, the neonate will exhibit congenital iodine, and therefore thyroid hormone, deficiency syndrome. Left untreated, the condition results in significant and irreversible deficits. A lack of iodine in the diet may have been the cause of the maternal hypothyroidism. Treated with oral thyroxine (T4) from birth to death. Accidental loss of the thyroid gland is likewise treated with hormone replacement therapy.

While the systemic circulation of immunosuppressants is generally accepted as increasing the recurrence of cancer after thyroidectomy, a large multicentric study failed to substantiate this (Tisset, H., Kamar, N., Faugeron, I., Roy, P., Pouteil-Noble, C., and 17 others 2013. “Is Thyroid Cancer Recurrence Risk Increased after Transplantation?,” Online, Journal of Clinical Endocrinology and Metabolism (New York, New York) 98(10):3981-3988). That an implanted automatic disorder response system can directly pipeline target immunosuppressives to substantially circumscribed locations may further ameliorate recurrence.

Thyroid carcinoma, at about 80 percent, primarily papillary, but also follicular, medullary, and anaplastic, together account for almost all cases of thyroid cancer, which almost always requires destruction of the native thyroid, whether through a total or lobal thyroidectomy, radioiodine ablation of any vestige, and uncommonly, external beam radiation, or chemotherapy, thus prompting consideration of a thyroid transplant (see, for example, U.S. National Cancer Institute Adult Treatment Editorial Board 2019. “Thyroid Cancer Treatment (Adult)(PDQ®) [Physician Data Query],” Online at https://www.nebi.nlm.nih.gov/books/NBK65719/; U.S. National Cancer Institute Pediatric Treatment Editorial Board 2019. “Childhood Thyroid Cancer Treatment (PDQ®) [Physician Data Query], Online at https://www.ncbi.nlm.nih.gov/books/NBK475809/; Chen, G. X., Li, C., and Zhang, H. 2019. “Drainage during Endoscopic Thyroidectomy,” Journal of the Society of Laparoendoscopic Surgeons 23(1). pii: e2018.00060; Sanyal, P., Mukherjee. T., Barui. S., Das. A., and Gangopadhyay, P. 2018. “Artificial Intelligence in Cytopathology: A Neural Network to Identify Papillary Carcinoma on Thyroid Fine-needle Aspiration Cytology Smears,” Journal of Pathology Informatics 9:43; Saini, S., Tulla, K., Maker, A. V., Burman, K. D., and Prabhakar, B. S. 2018. “Therapeutic Advances in Anaplastic Thyroid Cancer: A Current Perspective,” Molecular Cancer 17(1):154; Iyer, P. C., Dadu, R., Gule-Monroe, M., Busaidy, N. L., Ferrarotto, R., and 9 others 2018. “Salvage Pembrolizumab Added to Kinase Inhibitor Therapy for the Treatment of Anaplastic Thyroid Carcinoma,” Journal for Immunotherapy of Cancer 6(1):68; Qi, X. P., Peng, J. Z., Yang, X. W., Zao, Z. L., Yu, X. H., and 3 others 2018. “The RET [rearranged during transfection] C611Y Mutation Causes MEN 2A [multiple endocrine neoplasia type 2A] and Associated Cutaneous Lichen Amyloidosis,” Endocrine Connections 7(9):998-1005; Grimm, D. 2017. “Current Knowledge in Thyroid Cancer—From Bench to Bedside,” Online, MDPI [Multidisciplinary Digital Publishing Institute] International Journal of Molecular Sciences (Basel, Switzerland) 18(7). pii: E1529; Ferrari, S. M., La Motta, C., Elia, G., Ragusa, F., Ruffilli, I., and 9 others 2018. “Antineoplastic Effect of Lenvatinib and Vandetanib in Primary Anaplastic Thyroid Cancer Cells Obtained from Biopsy or Fine Needle Aspiration,” Online, Frontiers in Endocrinology (Lausanne, Switzerland) 9:764; Savala, R., Dey. P., and Gupta, N. 2018. “Artificial Neural Network Model to Distinguish Follicular Adenoma from Follicular Carcinoma on Fine Needle Aspiration of Thyroid,” Diagnostic Cytopathology 46(3):244-249; Cherian, A. J., Ramakant, P., Pai, R., Manipadam, M. T., Elanthenral, S., and 9 others 2018. “Outcome of Treatment for Medullary Thyroid Carcinoma-A Single Centre Experience,” Indian Journal of Surgical Oncology 9(1):52-58; Carney, J. A., Lyssikatos, C., Seethala, R. R., Lakatos, P., Perez-Atayde, A., Lahner, H., and Stratakis, C. A. 2018. “The Spectrum of Thyroid Gland Pathology in Camey Complex: The Importance of Follicular Carcinoma,” American Journal of Surgical Pathology 42(5):587-594; Cao, L. 2018. “Assessment of Thyroid Cancer Risk in More than 334,000 Patients with Inflammatory Bowel Disease: A Case-control Study and a Meta-analysis,” World Journal of Surgical Oncology 16(1):182; Kitahara, C. M., Yanik, E. L., Ladenson, P. W., Hernandez, B. Y., Lynch, C. F., Pawlish, K. S., and Engels, E. A. 2017. “Risk of Thyroid Cancer among Solid Organ Transplant Recipients,” American Journal of Transplantation 17(11):2911-2921; Kitahara, C. M., Yanik, E. L., Ladenson, P. W., Hernandez, B. Y., Lynch, C. F., Pawlish, K. S., and Engels, E. A. 2017. “Risk of Thyroid Cancer among Solid Organ Transplant Recipients,” American Journal of Transplantation 17(11):2911-2921; Iniguez-Ariza, N. M., Ryder, M. M., Hilger, C. R., and Bible, K. C. 2017. “Salvage Lenvatinib Therapy in Metastatic Anaplastic Thyroid Cancer,” Thyroid 27(7):923-927; Grimm, D. 2017. “Current Knowledge in Thyroid Cancer—From Bench to Bedside,” Online, MDPI [Multidisciplinary Digital Publishing Institute] International Journal of Molecular Sciences (Basel, Switzerland) 18(7). pii: E1529; Cheng, Q., Li, X., Acharya, C. R., Hyslop, T., and Sosa, J. A. 2017. “A Novel Integrative Risk Index of Papillary Thyroid Cancer Progression Combining Genomic Alterations and Clinical Factors,” Oncotarget 8(10):16690-16703; Padur, A. A., Kumar, N., Guru, A., Badagabettu, S. N., Shanthakumar, S. R., Virupakshamurthy, M. B., and Patil, J. 2016. “Safety and Effectiveness of Total Thyroidectomy and Its Comparison with Subtotal Thyroidectomy and Other Thyroid Surgeries: A Systematic Review,” Journal of Thyroid Research 2016:7594615; Opsahl, E. M., Brauckhoff, M., Schlichting, E., Helset, K., Svartberg. J., and 9 others 2016. “A Nationwide Study of Multiple Endocrine Neoplasia Type 2A in Norway: Predictive and Prognostic Factors for the Clinical Course of Medullary Thyroid Carcinoma,” Thyroid 26(9):1225-1238; Calvo, J., Torrealba, G., Saenz, A., Santamaria, C., Morera, E., Alvarado, S., Roa, Y., and González, M. 2016. “Genetic and Clinical Features of Medullary Thyroid Carcinoma: The Experience of a Single Center in Costa Rica,” Journal of Cancer Epidemiology 2016:9637173; Keutgen, X. M., Sadowski, S. M., and Kebebew, E. 2015. “Management of Anaplastic Thyroid Cancer,” Gland Surgery 4(1):44-51; Pelizzo, M. R., Torresan, F., Boschin, I. M., Nacamulli, D., Pennelli, G., and 3 others 2015. “Early, Prophylactic Thyroidectomy in Hereditary Medullary Thyroid Carcinoma: A 26-year Monoinstitutional Experience,” American Journal of Clinical Oncology 38(5):508-513; Nguyen, Q. T., Lee, E. J., Huang, M. G., Park, Y. I., Khullar, A., and Plodkowski, R. A. 2015. “Diagnosis and Treatment of Patients with Thyroid Cancer,” American Health and Drug Benefits 8(1):30-40; Enewold, L., Harlan, L. C., Stevens, J. L., and Sharon, E. 2015. “Thyroid [papillary] Cancer Presentation and Treatment in the United States,” Annals of Surgical Oncology 22(6):1789-1797; Shah, J. P. 2015. “Thyroid Carcinoma: Epidemiology, Histology, and Diagnosis,” Clinical Advances in Hematology and Oncology 13(4 Supplement 4):3-6; Maia, A. L., Siqueira, D. R., Kulcsar, M. A., Tincani, A. J., Mazeto, G. M., and Maciel, L. M. 2014. “Diagnosis, Treatment, and Follow-up of Medullary Thyroid Carcinoma: Recommendations by the Thyroid Department of the Brazilian Society of Endocrinology and Metabolism,” Arquivos Brasileiros de Endocrinologia e Metabologia (Brazilian Archives of Endocrinology and Metabolism) 58(7):667-700; Qi, X. P., Zhao, J. Q., Du Z. F., Yang, R. R., Ma, J. M., and 13 others 2013. “Prophylactic Thyroidectomy for MEN 2 [multiple endocrine neoplasia type 2]-related Medullary Thyroid Carcinoma Based on Predictive Testing for RET [rearranged during transfection] Proto-oncogene Mutation and Basal Serum Calcitonin in China,” European Journal of Surgical Oncology 39(9):1007-1012; Kiratli, P. O., Volkan-Salanci, B., Günay, E. C., Varan, A., Akyüz, C., and Büyükpamukcu, M. 2013. “Thyroid Cancer in Pediatric Age Group: An Institutional Experience and Review of the Literature,” Journal of Pediatric Hematology and Oncology 35(2):93-97; Enomoto, K., Enomoto, Y., Uchino, S., Yamashita, H., and Noguchi, S. 2013. “Follicular Thyroid Cancer in Children and Adolescents: Clinicopathologic Features, Long-term Survival, and Risk factors for Recurrence,” Endocrine Journal 60(5):629-635; Kunavisarut, T. 2013. “Diagnostic Biomarkers of Differentiated Thyroid Cancer,” Endocrine 44(3):616-622; Taccaliti, A., Silvetti, F., Palmonella, G., and Boscaro, M. 2012. “Anaplastic Thyroid Carcinoma,” Online, Frontiers in Endocrinology (Lausanne, Switzerland) 3:84; Adedapo, K. S., Fadiji, I. O., Orunmuyi, A. T., Onimode, Y., and Osifo, B. O. 2012. “Radioactive Iodine Ablation Therapy: A Viable Option in the Management of Graves' Disease in Nigeria,” African Journal of Medicine and Medical Sciences 41 Supplement: 193-196; Ibrahimpasic, T., Nixon, I. J., Palmer, F. L., Whitcher, M. M., Tuttle, R. M., and 4 others 2012. “Undetectable Thyroglobulin after Total Thyroidectomy in Patients with Low- and Intermediate-risk Papillary Thyroid Cancer—Is There a Need for Radioactive Iodine Therapy?,” Surgery 152(6):1096-1105; Perri, F., Lorenzo, G. D., Scarpati, G. D., and Buonerba, C. 2011. “Anaplastic Thyroid Carcinoma: A Comprehensive Review of Current and Future Therapeutic Options,” World Journal of Clinical Oncology 2(3):150-157; Nagaiah, G., Hossain, A., Mooney, C. J., Parmentier, J., and Remick, S. C. 2011. “Anaplastic Thyroid Cancer: A Review of Epidemiology, Pathogenesis, and Treatment,” Journal of Oncology 2011:542358; Burns, W. R. and Zeiger. M. A. 2010. “Differentiated Thyroid Cancer,” Seminars in Oncology 37(6):557-566; Chiacchio. S., Lorenzoni, A., Boni, G., Rubello, D., Elisei, R., and Mariani, G. 2008. “Anaplastic Thyroid Cancer: Prevalence, Diagnosis and Treatment,” Minerva Endocrinologica 33(4):341-357; Caron, N. R. and Clark, O. H. 2006. “Papillary Thyroid Cancer,” Current Treatment Options in Oncology 7(4):309-319).

Relatively low in incidence, the same may be said for a primary sarcomatous thyroid gland, whether affected by angiosarcoma, hemangioendothelioma, fibrous histiocytoma, leiomyosarcoma, fibrosarcoma, and liposarcoma (see, for example, Wei, J., Yang, J., Liang, W., Xu, C., and Wen, Y. 2019. “Clinicopathological Features of Primary Thyroid Leiomyosarcoma without Epstein-Barr Virus Infection: A Case Report,” Oncology Letters 17(1):281-287; Chen, Q., Huang, Q., Yan, J. X., Li, C., and Lang, J. Y. 2018. “Primary Undifferentiated Pleomorphic Sarcoma of the Thyroid: A Case Report and Review of the Literature,” Medicine (Baltimore, Maryland) 97(7):e9927; Palla, A. R., Bollig. C. A., and Jorgensen, J. B. 2018. “Well-differentiated Liposarcoma Localized to the Thyroid Gland,” European Thyroid Journal 7(5):262-266; Guarda, V., Pickhard, A., Boxberg, M., Specht. K., and Buchberger, A. M. S. 2018. “Liposarcoma of the Thyroid: A Case Report with a Review of the Literature,” European Thyroid Journal 7(2):102-108; Liu, J. Liu, X. Q. Yan, B. D. Xue, Y. J., Han, X. X., and 4 others 2018. “Pulmonary Multiple Nodules: Benign or Malignant?,” Chinese Medical Journal (English edition) 131(16):1999-2001; Kabata, P., Kaniuka-Jakubowska, S., Kabata, W., Lakomy, J., Biernat, W., and 3 others 2017. “Primary Ewing Sarcoma of the Thyroid-Eight Cases in a Decade: A Case Report and Literature Review,” Online, Frontiers in Endocrinology (Lausanne, Switzerland) 8:257; Surov. A., Gottschling, S., Wienke, A., Meyer, H. J., Spielmann, R. P., and Dralle, H. 2015. “Primary Thyroid Sarcoma: A Systematic Review,” Anticancer Research 35(10):5185-5191; Wang. T. S., Ocal. I. T., Oxley, K., and Sosa, J. A. 2008. “Primary Leiomyosarcoma of the Thyroid Gland,” Thyroid 18(4):425-428; Marteil-Oudrer, A. M., Friess, P., Vix, M., Gasser, B., Jeandidier, N., and Pinget, M. 2002. “Sarcoma of the Thyroid Gland: A Case Report,” Annales d' Endocrinologie (Paris, France) 63(3):219-222)

Thyroid hormone replacement poses problems with respect to proper formulation for a specific patient, and stem cell therapy should serve the needs of thyroid dysfunction only once the ability to culture functional thyroid cells becomes practicable (Davies, T. F. 2013. “Is Thyroid Transplantation on the Distant Horizon?,” Thyroid 23(2):139-141). In this circumstance, and given that the automatic and direct pipeline targeting to the graft thyroid of immunosuppressant medication by an implanted disorder response system would be a simple matter, heterotopic allotransplantation of a thyroid gland taken from a donor on life support offers a feasible and surgically approachable means for establishing normal thyroid function.

As to orthotopic replacement of the native thyroid, the intimate contact of the structures in the neck means that endoscopic thyroidectomy and replacement of the thyroid gland with parathyroids or with autografts of parathyroid tissue using a switch technique, requires especially compact valve-jackets to accomplish connection to the superior, inferior, and the infrequently encountered and highly variable lowest thyroid artery (thyroid artery of Neubauer, arteria thyroidea ima, thyroidea ima artery). A lowest artery may compensate for absence of any or all of the other thyroid arteries. The superior thyroid artery is more accessible than the inferior, which arises from the thyrocervical trunk, itself too short and overcrowded to provide a good and problem-free connection.

The thyroid and parathyroid glands self-regulatory, servovalves are usually not necessary on the thyroid supply and drainage vessels, a want of sufficient thyroid hormone or failure of hormone uptake compensated for with oral medication. Where the patient is incompetent for regimen compliance, a simple implanted response system with sensors to report thyroid hormone levels to a microcontroller prescription-programmed to command the outlet pump or drip stopcock of one or more small drug and iodine reservoirs positioned subdermally in the pectoral area and replenished through a small subdermally placed port close to it to provide an antithyroid drug or supplementary thyroid hormone as appropriate.

Using radiation-shielded lines as described and illustrated in U.S. Pat. No. 11,013,858, entitled Nonjacketing Side-entry Connectors and Prosthetic Disorder Response Systems, and U.S. Pat. No. 11,759,186, entitled Ductus Side-entry jackets and Prosthetic Disorder Response Systems, which describes and illustrates side-entry valve and servovalve-jackets, radioiodine can be directly pipeline targeted to the thyroid, avoiding exposure of other tissue.

Targeted radioiodine can be used therapeutically and/or diagnostically as contrast (see, for example, Liu, H., Wang, X., Yang, R., Zeng. W., Peng, D., Li, J., and Wang, H. 2018. “Recent Development of Nuclear Molecular Imaging in Thyroid Cancer,” Online, Biomed Research International 2018:2149532; Choudhury, P. S. and Gupta, M. 2018. “Differentiated Thyroid Cancer Theranostics: Radioiodine and Beyond” British Journal of Radiology 91(1091):20180136; Al Eyadeh, A. A., Al-Sarihin, K., Etewi, S., Al-Omari, A., Al-Asa'd, R. A., and Haddad, F. H. 2017. “Thyroid Cancer Post Radioactive Iodine Treatment for Hyperthyroidism-Case Series and Review of the Literature,” Endokrynologia Polska 68(5):561-566; Santhanam, P., Solnes, L. B., and Rowe, S. P. 2017. “Molecular Imaging of Advanced Thyroid Cancer: Iodinated Radiotracers and Beyond,” Medical Oncology (Northwood, London, England) 34(12):189; Castagna, M. G., Cantara, S., and Pacini, F. 2016. “Reappraisal of the Indication for Radioiodine Thyroid Ablation in Differentiated Thyroid Cancer Patients,” Journal of Endocrinolological Investigation 39(10):1087-1094; Volkan-Salancι, B. and Kiratlι, P. O. 2015. “Nuclear Medicine in Thyroid Diseases in Pediatric and Adolescent Patients,” Molecular Imaging and Radionuclide Therapy 24(2):47-59; Schneider, D. F., Sonderman, P. E., Jones, M. F., Ojomo, K. A., Chen, H., and 4 others 2014. “Failure of Radioactive Iodine in the Treatment of Hyperthyroidism,” Annals of Surgical Oncology 21(13):4174-4180; Nwatsock, J. F., Taieb, D., Tessonnier. L., Mancini, J., Dong-A-Zok, F., and Mundler, O. 2012. “Radioiodine Thyroid Ablation in Graves' Hyperthyroidism: Merits and Pitfalls,” World Journal of Nuclear Medicine 11(1):7-11; Adedapo, K. S., Fadiji, I. O., Orunmuyi. A. T., Onimode, Y., and Osifo, B. O. 2012, Op cit.).

Antithyroid drugs pose risks of side effects ranging from migrating polyarthritis to erythema nodosum and are recommended for directly pipeline targeted delivery by an automatic disorder control system as a ‘fine adjustment’ to a substantially well-functioning thyroid allotransplant. Drugs used to treat hyperthyroidism are well established, and automatic administration by an implanted disorder response system will eliminate the risks associated with drug interactions of antithyroid drugs.

The avoidance of such complications, along with the directly targeted release capability of the fully implanted automatic disorder control system, is an important argument for the automatic administration of drugs in general. The automatic release ability of the control system is applicable to the treatment of hyperthyroidism apart from transplantation (see, for example, Allam, M. M., El-Zawawy, H. T., Kader Okda, A. A., Ali Alshaikh, A., and Ghazy, R. M. 2023. “Azathioprine as an Adjuvant Therapy in Severe Graves' Disease: A Randomized Controlled Open-label Clinical Trial,” Online, Frontiers in Endocrinology Lausanne, Switzerland) 14:1168936; Karmisholt, J., Andersen, S. L., Bulow-Pedersen, I., Carle, A., Krejbjerg, A., and Nygaard, B. 2019. “Predictors of Initial and Sustained Remission in Patients Treated with Antithyroid Drugs for Graves' Hyperthyroidism: The RISG [Remission Induction and Sustenance in Graves' Disease] Study,” Online, Journal of Thyroid Research (New York, New York) 2019:5945178; Torring, O., Watt. T., Sjolin, G., Bystrom, K., Abraham-Nordling, M., and 8 others 2019. “Impaired Quality of Life after Radioiodine Therapy Compared with Antithyroid Drugs or Surgical Treatment for Graves' Hyperthyroidism. A Long-term Follow-up with ThyPRO and SF-36,” Thyroid available Online at https://doi.org/10.1089/thy.2018.0315; Gershinsky, M., Saliba, W., Lavi, I., Shapira, C., and Gronich, N. 2019. “Increased Risk of Antithyroid Drugs Agranulocytosis Associated with Amiodarone-induced Thyrotoxicosis: A Population-based Cohort Study,” Thyroid available Online at https://www.nebi.nlm.nih.gov/m/pubmed/30648930/; Cole, M., Hynes, A. M., Howel, D., Hall. L., Abinun, M., and 9 others 2019. “Adjuvant Rituximab, a potential treatment for the Young Patient with Graves' Hyperthyroidism (RiGD): Study Protocol for a Single-arm, Single-stage, Phase II Trial,” Online, British Medical Journal Open 9(1):e024705; Kim. H., Lee, J., and Ha, J. 2018. “A Case of Antithyroid Drug-induced Agranulocytosis from a Second Antithyroid Drugs (ATD) Administration in a Relapsed Graves' Disease Patient Who Was Tolerant to the First ATD Treatment” Clinical Case Reports 6(9):1701-1703; Brammen, L., Riss, P., Lukas, J., Gessl, A., Dunkler, D., and 7 others 2018. “Total Thyroidectomy (Tx) versus Thionamides (Antithyroid Drugs) in Patients with Moderate-to-severe Graves' Ophthalmopathy—A 1-year Follow-up: Study Protocol for a Randomized Controlled Trial,” Online, BioMed Central Trials (London, England) 19(1):495; Andrade Luz, I., Pereira. T., and Catorze, N. 2018. “Thyroid Storm: A Case of Haemodynamic Failure Promptly Reversed by Aggressive Medical Therapy with Antithyroid Agents and Steroid Pulse,” Online, British Medical Journal Case Reports (London, England) 11(1) pi: e226669; Huang, H., Shi, Y., Liang. B., Cai. H., Cai, Q., and Lin, R. 2018. ‘Optimal Iodine Supplementation during Antithyroid Drug Therapy for Graves’ Disease is Associated with Lower Recurrence Rates than Iodine Restriction,” Clinical Endocrinology (Oxford, England) 88(3):473-478; Liu, J., Fu, J., Xu. Y., and Wang, G. 2017. “Antithyroid Drug Therapy for Graves' Disease and Implications for Recurrence,” International Journal of Endocrinology 2017:3813540; Azizi, F. and Malboosbaf. R. 2017. “Long-term Antithyroid Drug Treatment: A Systematic Review and Meta-Analysis,” Thyroid 27(10):1223-1231; Vicente, N., Cardoso, L., Barros, L., and Carrilho, F. 2017. “Antithyroid Drug-induced Agranulocytosis: State of the Art on Diagnosis and Management,” Drugs in Research and Development 17(1):91-96; Hesarghatta Shyamasunder, A. and Abraham, P. 2017. “Measuring TSH [thyroid stimulating hormone] Receptor Antibody to Influence Treatment Choices in Graves' Disease,” Clinical Endocrinology (Oxford, England) 86(5):652-657; Kiriyama, H., Amiya, E., Hatano, M., Hosoya, Y., Maki, H., and 11 others 2017. “Rapid Improvement of Thyroid Storm-related Hemodynamic Collapse by Aggressive Anti-thyroid Therapy Including Steroid Pulse: A Case Report,” Medicine (Baltimore, Maryland) 96(22):e 7053; Takaya, K., Kimura, N., and Hiyoshi, T. 2016. “Antithyroid Arthritis Syndrome: A Case Report and Review of the Literature,” Internal Medicine (Tokyo, Japan) 55(24):3627-3633; Ma, C., Xie, J., Wang, H., Li, J., and Chen, S. 2016. “Radioiodine Therapy versus Antithyroid Medications for Graves' Disease,” Cochrane Database of Systematic Reviews 2: CD010094; Janson, J. A., de Laat. P., and Draaisma, J. M. 2015. Migratory Polyarthritis as an Adverse effect of Thiamazole Use in a 13-year-old Girl with Graves' Disease,” Journal of Pediatric Endocrinology and Metabolism 28(9-10):1169-1171; Balavoine, A. S., Glinoer, D., Dubucquoi, S., Werneau, J. L. 2015. “Antineutrophil Cytoplasmic Antibody-positive Small-vessel Vasculitis Associated with Antithyroid Drug Therapy: How Significant is the Clinical Problem?,” Thyroid 25(12):1273-1281; Jastrzębska, H. 2015. “Antithyroid Drugs,” Thyroid Research 8 (Supplement 1):A12, published Online at https://www.nebi.nlm.nih.gov/pme/articles/PMC4480840/; Laurberg, P., Krejbjerg, A., and Andersen, S. L. 2014. “Relapse following Antithyroid Drug Therapy for Graves' Hyperthyroidism,” Current Opinion in Endocrinology, Diabetes, and Obesity 21(5):415-421; Klubo-Gwiezdzinska, J. and Wartofsky, L. 2012. “Thyroid Emergencies,” Medical Clinics of North America 96(2):385-403; Taylor, P. N. and Vaidya, B. 2012. “Side Effects of Anti-thyroid Drugs and Their Impact on the Choice of Treatment for Thyrotoxicosis in Pregnancy,” European Thyroid Journal 1(3):176-185; Azizi, F. 2006. “The Safety and Efficacy of Antithyroid Drugs,” Expert Opinion on Drug Safety 5(1):107-116; Cooper, D. S. 2005. “Antithyroid Drugs,” New England Journal of Medicine 352(9):905-917).

Supplementary thyroid hormone therapy is used to treat hypothyroidism (see, for example, Soh, S. B. and Aw. T. C. 2019. “Laboratory Testing in Thyroid Conditions-Pitfalls and Clinical Utility,” Annals of Laboratory Medicine 39(1):3-14; Jonklaas, J., Tefera, E., and Shara, N. 2019. “Prescribing Therapy for Hypothyroidism: Influence of Physician Characteristics,” Thyroid 29(1):44-52; Solter, D. and Solter, M. 2018. “Decreased Sensitivity of Thyrotropin-Thyroid Hormone Feedback Control in Hypothyroid and Athyreotic Patients Treated with Levothyroxine. Is Serum Triiodothyronine Involved?,” Annales d' Endocrinologie (Paris, France) 79(2):62-66; Dayan, C. and Panicker, V. 2018. “Management of Hypothyroidism with Combination Thyroxine (T4) and Triiodothyronine (T3) Hormone Replacement in Clinical Practice: A Review of Suggested Guidance,” Thyroid Research 11:1; Hoermann, R., Midgley, J. E. M., Larisch, R., and Dietrich, J. W. 2018. “Lessons from Randomised Clinical Trials for Triiodothyronine Treatment of Hypothyroidism: Have They Achieved Their Objectives?,” Journal of Thyroid Research 2018:3239197; Schneider, C., Feller, M., Bauer, D. C., Collet, T. H., da Costa, B. R., and 10 others 2018. “Initial Evaluation of Thyroid Dysfunction—Are Simultaneous TSH and fT4 Tests Necessary?,” Online, PLoS [Public Library of Science] One 13(4):e0196631; Jonklaas, J., Tefera, E., and Shara, N. 2018. “Physician Choice of Hypothyroidism Therapy: Influence of Patient Characteristics,” Thyroid 28(11):1416-1424; Tariq. A., Wert. Y., Virili, C., Giovanella, L., Fallahi, P., Antonelli, A., Santaguida, M. G., Centanni, M., and Trimboli, P. 2018. “Levothyroxine Therapy: Changes of TSH Levels by Switching Patients from Tablet to Liquid Formulation. A Systematic Review and Meta-Analysis,” Online, Frontiers in Endocrinology (Lausanne, Switzerland) 9:10; Cheriyath, P., and Joshi. R. 2018. “Effects of Long-term Combination LT4 and LT3 [levothyroxine] Therapy for Improving Hypothyroidism and Overall Quality of Life,” Southern Medical Journal 111(6):363-369; Hoermann, R., Midgley, J. E. M., Larisch, R.1, and Dietrich, J. W. 2018. “The Role of Functional Thyroid Capacity in Pituitary Thyroid Feedback Regulation,” European Journal of Clinical Investigation 48(10):e13003; McAninch, E. A., Rajan, K. B., Miller, C. H., and Bianco, A. C. 2018. “Systemic Thyroid Hormone Status during Levothyroxine Therapy in Hypothyroidism: A Systematic Review and Meta-analysis,” Journal of Clinical Endocrinology and Metabolism August 15; Perros, P. 2018. “A Decade of Thyroidology,” Hormones (Athens, Greece) 17(4):491-495; van den Beld. A. W., Kaufman, J. M., Zillikens, M. C., Lamberts, S. W. J., Egan, J. M., and van der Lely, A. J. 2018. “The Physiology of Endocrine Systems with Ageing,” Lancet, Diabetes and Endocrinology (London, England) 6(8):647-658; Hennessey, J. V. and Espaillat, R. 2018. “Current Evidence for the Treatment of Hypothyroidism with Levothyroxine/Levotriiodothyronine Combination Therapy versus Levothyroxine Monotherapy,” International Journal of Clinical Practice 72(2); Michaelsson, L. F., la Cour, J. L., Medici, B. B., Watt. T., Faber, J., and Nygaard. B. 2018. “Levothyroxine/Liothyronine Combination Therapy and Quality of Life: Is It All about Weight Loss?,” European Thyroid Journal 7(5):243-250; Berberich, J., Dietrich, J. W., Hoermann, R., and Müller, M. A. 2018. “Mathematical Modeling of the Pituitary-Thyroid Feedback Loop: Role of a TSH-T3-Shunt and Sensitivity Analysis,” Online, Frontiers in Endocrinology (Lausanne, Switzerland) 9:91; Park, E., Jung, J., Araki, O., Tsunekawa, K., Park, S. Y., and 4 others 2018. “Concurrent TSHR [thyroid stimulating hormone receptor] Mutations and DIO2 T92A Polymorphism Result in Abnormal Thyroid Hormone Metabolism,” (Nature Research) Scientific Reports 8(1):10090; Samuels, M. H., Kolobova, 1.2., Niederhausen, M., Purnell, J. Q., and Schuff, K. G. 2018. “Effects of Altering Levothyroxine Dose on Energy Expenditure and Body Composition in Subjects Treated with LT4 [levothyroxine],” Journal of Clinical Endocrinology and Metabolism 103(11):4163-4175; Hoermann, R., Midgley, J. E. M., Dietrich, J. W., and Larisch, R. 2017. “Dual Control of Pituitary Thyroid Stimulating Hormone Secretion by Thyroxine and Triiodothyronine in Athyreotic Patients,” Therapeutic Advances in Endocrinology and Metabolism 8(6):83-95; Wiersinga, W. M. “Therapy of Endocrine Disease: T4+T3 Combination Therapy: Is There a True Effect?,” European Journal of Endocrinology 2017 177(6):R287-R296; Samuels, M. H., Kolobova, I., Antosik, M., Niederhausen, M., Purnell, J. Q., Schuff, K. G. 2017. “Thyroid Function Variation in the Normal Range, Energy Expenditure, and Body Composition in L-T4-treated Subjects,” Journal of Clinical Endocrinology and Metabolism 102(7):2533-2542; Gullo, D., Latina, A., Frasca, F., Squatrito, S., Belfiore, A., and Vigneri, R. 2017. “Seasonal Variations in TSH Serum Levels in Athyreotic Patients under L-thyroxine Replacement Monotherapy,” Clinical Endocrinology (Oxford, England) 87(2):207-215; Ito. M., Miyauchi, A., Hisakado, M., Yoshioka, W., Ide. A., and 10 others 2017. “Biochemical Markers Reflecting Thyroid Function in Athyreotic Patients on Levothyroxine Monotherapy,” Thyroid 27(4):484-490; Goede, S. L., Latham, K. R., Leow, M. K., and Jonklaas, J. 2017. “High Resolution Free Triiodothyronine-Thyrotropin (FT3-TSH) Responses to a Single Oral Dose of Liothyronine in Humans: Evidence of Distinct Inter-individual Differences Unraveled Using an Electrical Network Model,” Journal of Biological Systems 25(1):119-143; Jonklaas, J. 2017. “Persistent Hypothyroid Symptoms in a Patient with a Normal Thyroid Stimulating Hormone Level,” Current Opinion in Endocrinology, Diabetes, and Obesity 24(5):356-363; Mammen, J. S., McGready, J., Ladenson, P. W., and Simonsick, E. M. 2017. “Unstable Thyroid Function in Older Adults is Caused by Alterations in Both Thyroid and Pituitary Physiology and is Associated with Increased Mortality,” Thyroid 27(11):1370-1377; Hoermann, R., Midgley, J. E. M., Larisch, R., and Dietrich, J. W. 2017. “Recent Advances in Thyroid Hormone Regulation: Toward a New Paradigm for Optimal Diagnosis and Treatment,” Online, Frontiers in Endocrinology (Lausanne, Switzerland) 8:364; Hennessey, J. V. 2017. “The Emergence of Levothyroxine as a Treatment for Hypothyroidism,” Endocrine 55(1):6-18; Jonklaas, J. 2016. “Update on the Treatment of Hypothyroidism,” Current Opinion in Oncology 28(1):18-25; Welsh, K. J. and Soldin, S. J. 2016. “Diagnosis of Endocrine Disease: How Reliable are Free Thyroid and Total T3 Hormone Assays?,” European Journal of Endocrinology 175(6):R255-R263; Hoermann, R., Midgley, J. E., Larisch, R., and Dietrich, J. W. 2016. “Relational Stability in the Expression of Normality, Variation, and Control of Thyroid Function,” Online, Frontiers in Endocrinology (Lausanne, Switzerland) 7:142; Hoermann, R., Midgley, J. E., Larisch, R., and Dietrich, J. W. 2016. “Relational Stability of Thyroid Hormones in Euthyroid Subjects and Patients with Autoimmune Thyroid Disease,” European Thyroid Journal 5(3):171-179; Han, C., Xia, X., Liu. A., Zhang, X., Zhou, M., and 6 others 2016. “Circulating Betatrophin is Increased in Patients with Overt and Subclinical Hypothyroidism,” Biomedical Research International 2016:5090852; Paone, L., Fleisch, A. F., Feldman, H. A., Brown, R. S., and Wassner, A. J. 2016. “Liothyronine Improves Biochemical Control of Congenital Hypothyroidism in Patients with Central Resistance to Thyroid Hormone,” Journal of Pediatrics 175:167-172.e1; Ortiga-Carvalho, T. M., Chiamolera, M. I., Pazos-Moura, C. C., and Wondisford, F. E. 2016. “Hypothalamus-Pituitary-Thyroid Axis,” Comprehensive Physiology 6(3):1387-1428; Guglielmi, R., Frasoldati, A., Zini, M., Grimaldi, F., Gharib, H., Garber, J. R., and Papini, E. 2016. Italian Association of Clinical Endocrinologists Statement-Replacement Therapy for Primary Hypothyroidism: A Brief Guide for Clinical Practice,” Endocrine Practice 22(11):1319-1326; Kaminski, J., Miasaki, F. Y., Paz-Filho, G., Graf. H., and Carvalho, G. A. 2016. “Treatment of Hypothyroidism with Levothyroxine Plus Liothyronine: A Randomized, Double-blind, Crossover Study,” Archives of Endocrinology and Metabolism 60(6):562-572; Jonklaas, J. 2016. “Risks and Safety of Combination Therapy for Hypothyroidism,” Expert Review of Clinical Pharmacology 9(8):1057-1067; Jonklaas, J. and Burman, K. D. 2016. “Daily Administration of Short-acting Liothyronine is Associated with Significant Triiodothyronine Excursions and Fails to Alter Thyroid-responsive Parameters,” Thyroid 26(6):770-778; Livingston, M., Birch, K., Guy, M., Kane, J., and Heald, A. H. 2015. “No Role for Tri-iodothyronine (T3) Testing in the Assessment of Levothyroxine (T4) Over-replacement in Hypothyroid Patients,” British Journal of Biomedical Science 72(4):160-163; Hoermann, R., Midgley, J. E., Larisch, R., and Dietrich, J. W. 2015. “Homeostatic Control of the Thyroid-Pituitary Axis: Perspectives for Diagnosis and Treatment,” Online, Frontiers in Endocrinology (Lausanne, Switzerland) 6:177; Ito. M., Miyauchi. A., Kang. S., Hisakado, M., Yoshioka, W., and 10 others 2015. “Effect of the Presence of Remnant Thyroid Tissue on the Serum Thyroid Hormone Balance in Thyroidectomized Patients,” European Journal of Endocrinology 173(3):333-340; Jonklaas, J., Burman, K. D., Wang. H., and Latham, K. R. 2015. “Single-dose T3 Administration: Kinetics and Effects on Biochemical and Physiological Parameters,” Therapeutic Drug Monitoring 37(1):110-118; Geer, M., Potter, D. M., and Ulrich, H. 2015. “Alternative Schedules of Levothyroxine Administration,” American Journal of Health-System Pharmacy 72(5):373-377; Kraut, E and Farahani, P. 2015. “A Systematic Review of Clinical Practice Guidelines' Recommendations on Levothyroxine Therapy Alone versus Combination Therapy (LT4 plus LT3) for Hypothyroidism,” Clinical and Investigative Medicine 38(6):E305-E313; Ala, S., Akha, O., Kashi, Z., Asgari, H., Bahar, A., and Sasanpour, N. 2015. “Does Administration Time from Before Breakfast to Before Dinner Affect Thyroid Hormone Levels?,” Caspian Journal of Internal Medicine Summer 6(3):134-140; Bagattini. B., Cosmo, C. D., Montanelli, L., Piagg. P., Ciampi. M., and 4 others 2014. “The Different Requirement of L-T4 Therapy in Congenital Athyreosis Compared with Adult-acquired Hypothyroidism Suggests a Persisting Thyroid Hormone Resistance at the Hypothalamic-Pituitary Level,” European Journal of Endocrinology 171(5):615-621; Jonklaas, J., Bianco, A. C., Bauer, A. J., Burman, K. D., Cappola, A. R., and 6 others 2014. “Guidelines for the Treatment of Hypothyroidism: Prepared by the American Thyroid Association Task Force on Thyroid Hormone Replacement,” Thyroid 24(12):1670-1751; Wartofsky, L. 2013. “Combination L-T3 and L-T4 Therapy for Hypothyroidism,” Current Opinion in Endocrinology, Diabetes, and Obesity 20(5):460-466; Colucci, P., Yue, C. S., Ducharme, M., and Benvenga, S. 2013. “A Review of the Pharmacokinetics of Levothyroxine for the Treatment of Hypothyroidism,” European Endocrinology 9(1):40-47; Chakera, A. J., Pearce, S. H., and Vaidya, B. 2012. “Treatment for Primary Hypothyroidism: Current Approaches and Future Possibilities,” Drug Design, Development, and Therapy 6:1-11; Khandelwal. D. and Tandon, N. 2012. “Overt and Subclinical Hypothyroidism: Who to Treat and How,” Drugs 72(1):17-33; Almandoz, J. P. and Gharib. H. 2012. Hypothyroidism: Etiology, Diagnosis, and Management,” Medical Clinics of North America 96(2):203-221; Gullo, D., Latina, A., Frasca, F., Le Moli, R., Pellegriti, G., and Vigneri, R. 2011. “Levothyroxine Monotherapy Cannot Guarantee Euthyroidism in All Athyreotic Patients,” Online, PLoS [Public Library of Science] One 6(8):e22552; Liewendahl, K. 1983. “Assessment of Thyroid Function with Hormone Assays,” (in English) Annales Chirurgiae et Gynaecologie Fenniae [Annals of Surgery and Gynecology of Finland] (Helsinki, Finland) [72(3):84-89; Kabadi, U. M. 1982. “Laboratory Tests for Evaluating Thyroid Therapy,” American Family Physician 26(3):183-188).

Due to the compactness of the structures in the neck and the facts that 1. Provided it is secure and functions normally, the location of the thyroid gland appears unimportant, 2. Surgery close to the recurrent laryngeal nerves makes surgery susceptible to serious injury, 3. The surgery is intricate, in a neonate, microsurgical, demanding of skill limited to large tertiary centers, and 4. Positioning of the graft in a location that offers more space allows the use of larger valve-jackets, eliminating the need for a binocular microscope, allotransplantation of the thyroid gland is best heterotopic. The connections joining the thyroid arteries and veins of the deceased donor having been kept on life support at one end to those of the recipient at the other are made through synthetic lines, usually ordinary catheter tubing, joined to the vessels of donor and recipient by diversion valve-jackets.

Compound vascular bypass transplantation is effectuated by seamlessly transferring the donor graft organ from the circulatory system of the donor into that of the recipient before the graft organ is harvested from the donor or—in an orthotopic heart or lung transplant, for example, the native organ is removed from the recipient. As depicted for an exigent sudden-switch compound vascular bypass orthotopic heart transplant and in FIGS. 18A thru 19B, and in FIGS. 20A thru 21C for a metered compound vascular bypass orthotopic heart transplant, the sequences of steps depicted are the same for a switch transplantation of any organ or gland. If the thyroid arteries and veins of the recipient are diseased, those of the donor can be taken together with the gland proper, or if those of neither donor or recipient are healthy, then, because the accessory channels of the valve-jackets allow the direct delivery of anticlotting and other drugs from a tiny port at the body surface of the recipient, synthetic tubing can replace the unwanted vessels or segments thereof; the same tubing with valve-jackets used to accomplish the transplantation are implanted with the graft gland as a permanent prosthesis.

Small valve-jackets are intended to minimize a more projected shape that would encroach upon neighboring tissue, thus allowing heterotopic positioning of the replacement thyroid in more locations. In confined spaces, except where the operator must have access to the valve surface, which pertains to one-time nonservovalve permanently switched or prosthesis-type valves, the valve-jackets are additionally provided with a cushioned surface. If necessary, a pinch of tissue is removed from a subjacent muscle such as the anterior scalene to make room.

These problems of confined space are eliminated when transplantation of the thyroid gland is heterotopic. Currently the intact thyroid is not taken from a donor on life support but instead either the thyroid is allografted heterotopically, eliminating the problem of insufficient room (Sakr, M., El-Kerm, Y., Abo-Elwafa, W., Mahmoud. A., and Fathi, I. 2018. Op cit., this section; Sakr, M. and Mahmoud. A. 2017, Op cit., this section Mohsen, A. A., Nada, A. A., Ibrahim, M. Y., Ghaleb, A. H., Abou-Gabal, M. A., Mohsen, A. A., and Wassef, A. T. 2017, Op cit., this section; Roy, P. G., Saund, M. S., Thusoo, T. K., Roy, D., and Sankar, R. 2003. “Fate of Human Thyroid Tissue Autotransplants,” Surgery Today 33(8):571-576), or stem cells are introduced to mature into functional thyroid cells over time (Kurmann, A. A., Serra, M., Hawkins, F., Rankin, S. A., Mori, M., and 12 others 2015. “Regeneration of Thyroid Function by Transplantation of Differentiated Pluripotent Stem Cells,” Cell Stem Cell 17(5):527-542; Davies, T. F. 2013. “Is Thyroid Transplantation on the Distant Horizon?,” Thyroid 23(2):139-141). Valve- jackets are essential to accomplish switch transplantation of the thyroid, not to adjust blood flow thereafter.

Were the thyroid not transplanted, basic ductus side-entry jackets would serve the purpose, to include providing one or more accessory channels as necessary (see, for example, Sitges-Serra, A., Lorente-Poch, L., and Sancho, J. 2018. “Parathyroid Autotransplantation in Thyroid Surgery,” Langenbecks Archives of Surgery 403(3):309-315; Perros, P. 2018, Op cit.; Barczyński, M., Golkowski, F., and Nawrot, I. 2017. “Parathyroid Transplantation in Thyroid Surgery,” Gland Surgery 6(5):530-536; Krausz, M. M., Ashkenazi, I., and Alfici, R. 2017. “Parathyroid Autotransplantation in Adults and Children,” (in Hebrew with abstract in English at Pubmed), Harefuah [The Recovery] (Tel Aviv, Israel) 156(3):167-170).

2b(1)(b)(b12). Metered Compound Vascular Bypass, or Switched-Organ Vascularized Composite Tissue Allotransplantation

Ischemia-free compound vascular bypass allotransplantation which eliminates ischemia-reperfusion injury should improve outcomes and do so materially. As with solid organ transplantation using the compound vascular bypass technique, vascularized composite tissue allotransplants are limited to those which have never undergone a period of no blood flow.

The preferred site for performing such a transplant is a deceased donor sustainment center staffed by experienced anesthesiologists and internists as described above in section 2a, entitled Background of the Invention. General. Next best is a smaller regional section in a smaller medical center specialized to carry out sustainment treatment, and least preferred for sustaining a graft of any kind is a mechanical transport system whether normothermic, hypothermic, or hyperthermic.

The applicability of compound vascular bypass transplantation to a vascularized composite allograft, or composite tissue allotransplantation, such as the face depends upon the accessibility of blood supply and drainage vessels which will support the entire graft that can be valved short of the donor and recipient vessel stump ends needed to allow their direct anastomosis, and the micromachinability of valves of the tiny size that may be required. Once optimized, transplantation of the face will still have to confront the fact that severe trauma of the face often includes irreparable damage to the eyes, which will continue to elude successful transplantation. The prospects for total eye transplantation are addressed above in section 2b(1)(b)(b12), entitled Sudden Compound Vascular Bypass, or Sudden Switched-organ, Transplantation.

As addressed at the end of section 2b(2)(1), entitled Compound vascular bypass Transplantation-associated and Unassociated Anastomosis Bypasses, when the graft provides corresponding donor-recipient articulable vessels but these cannot be left with the graft, these are used for metered transfer and when the vessels are anastomosed, the connectors are removed. When such vessels are lacking, reperfusion injury is minimized if not eliminated by positioning the donor and recipient side by side and implanting the graft immediately following harvesting.

Transplantation of the eyes is briefly addressed above in section 2b(1)(a), entitled Sudden Compound Vascular Bypass, or Sudden Switched-organ, Transplantation. Unlike heart transplantation, where a compound vascular bypass procedure eliminates the considerable trauma inflicted upon the graft organ, with a composite tissue allotransplantation of the face or a hand, for example, the dissection involved in harvesting the graft and to suture the graft onto the recipient eliminates this benefit. However, the absence of even a momentary interruption in perfusion still applies as a critical factor to foster long-term graft durability.

To pose negligible weight and not encroach upon neighboring tissue, valves in the face as elsewhere in the body are best made small in size as possible of light plastic metal and provided with an accessory channel, or drug feedline for the fully implanted prosthetic disorder response system to periodically drip through an anticoagulant in accordance with its prescription-program. As described below in this section, where to allow valves and not just a drug feedline to remain in place would produce a cosmetically unacceptable result, the corresponding donor-recipient vascular anastomoses and nerve connections having been established, the sequence of steps depicted in FIGS. 22A and 22B is continued by removing the bypassing bloodlines and switching the valves to cause flow through the anastomoses.

Unless retention of the valves causes discomfort, to leave these in place offers the benefit of allowing the anastomoses to be bypassed to allow healing and the release of drugs through the accessory channels without a constant flow of blood to quickly flush these away. If necessary, once a procedure has been completed, the vascular valves or servovalves can be replaced with lighter, smaller, and less costly simple diversion valves addressed in U.S. Pat. No. 11,759,186, entitled Ductus Side-entry Jackets and Prosthetic Disorder Response Systems.

Vascularized composite tissue switch allotransplantation differs from internal organ switch transplantation in that the graft, especially a replacement face, at the body surface, must be as cosmetically acceptable as can be achieved, without a visible protrusion of valves allowed to continue past the period for healing when swelling and disfiguring pending healing is considerable in any event. Fortunately, the main blood supply and drainage to the face course through the neck where the conformation of the patient may allow valves to be subdued in prominence using fat and/or filler.

As life changing, full facial transplantation “has generated ethical concerns about the exposure of otherwise young and healthy individuals to the sequelae of lifelong, high-dose, multidrug immunosuppression,” (Khalifian, S., Brazio, P. S., Mohan, R, Shaffer, C., Brandacher, G., Barth, R. N., and Rodriguez, E. D., 2014, Op cit., above in section 2a(3), entitled Remotely Procured Graft Organ Preservation). Critically, the function of a fully implanted automatic disorder response system fundamentally changes this eventuality through the automatic directly pipe targeted intermittent delivery to the supply and facial vessels of the face of immunosuppressants, steroids, and antimicrobials, for example, in relatively higher concentrations, thus avoiding the systemic dispersion of these as problematic for adverse side effects.

To expedite the attainment of an esthetically acceptable outcome, the tiny vascular servovalves with accessory channels having druglines used to accomplish the metered compound vascular bypass procedure are positioned subcutaneously behind the submandibular salivary glands. Such valves are applied to the facial artery, the ophthalmic artery once it exits the skull, and the facial, supratrochlear, and vein, and superficial temporal veins (see for example, Onishi, S., Imanishi, N., Yoshimura, Y., Inoue, Y., Sakamoto, Y., Chang, H., and Okumoto, T. 2017. “Venous Drainage of the Face,” Journal of Plastic, Reconstructive, and Aesthetic Surgery (Amsterdam, North Holland, Netherlands) 70(4):433-440; additional references cited just below and in the following subsection entitled Vascularized Composite Tissue Allotransplantation: The Axis-proximate, or Adaxial, Principle for Extremity Replacement).

To avoid synkineses and the need for follow-up selective neurolysis, neurectomy, or myectomy, and phantom phenomena, which cortical, remain to be treatable other than through peripheral nerve block, although the facial nerves must be joined in a discriminatory manner at the proper levels with any lost facial muscles replaced, after which phantom referrals may still pose a problem (see, for example, Anggelia, M. R., Cheng, H. Y., Lai, P. C., Hsieh, Y. H., Lin, C. H., and Lin C. H. 2022. “Cell Therapy in Vascularized Composite Allotransplantation,” Online, Biomedical Journal 45(3):454-464; He, J., Khan, U. Z., Qing, L, Wu, P., and Tang, J. 2022. “Improving the Ischemia-reperfusion Injury in Vascularized Composite Allotransplantation: Clinical Experience and Experimental Implications,” Online, Frontiers in Immunology (Lausanne, Switzerland) 13:998952; Kattner, A A. 2022. “A Finger in Every Pie—The Versatility of Chemokines,” Online, Biomedical Journal 45(3):427-431; Khajuria, A., Bisase, B., Norris, P., Dhanda, J., Koshima, I., Nduka, C., and Kannan, R. Y., 2022. “Facial Nerve Revascularization Strategies in Facial Restoration,” Plastic and Reconstructive Surgery Global Open 10(1):4038; Lellouch, A. G., Andrews, A. R., Saviane. G., Ng, Z. Y., Schol, I. M., and 8 others 2022. “Tolerance of a Vascularized Composite Allograft Achieved in MHC [major histocompatibility complex] Class-1-mismatch Swine via Mixed Chimerism,” Online, Frontiers in Immunology (Lausanne, Switzerland) 13:829406; Lei. B., Sleiman, M. M., Cheng, Q., Tu, Z. Zhu, P., and 6 others 2021. Op cit., above in section 2a, entitled Background. General; Matos Cruz, A. J. and De Jesus, O. 2021. “Facial Nerve Repair,” Online, Treasure Island, Florida: StatPearls Publishing Co.; Savkliyildiz, A., Ozkan, O., Uysal, H., Senol, U., Ozkan, O., and 9 others 2021. “Adaptive Analysis of Cortical Plasticity with fMRI (functional magnetic resonance imaging) in Full-face and Army Transplants,” Brain Imaging and Behavior 15(4):1788-1801; Seneviratne, S. O. and Patel, B. C. 2021. “Facial Nerve Anatomy and Clinical Applications,” Online, Treasure Island, Florida: StatPearls Publishing Co.; Uysal, H., Savkliyildiz, A., Ozkan, O., Colak, O. H., Barcin, E., and 3 others 2021. “Complementary Phenomena: Phantom Hand and Phantom Face,” Cognitive and Behavioral Neurology 34(2):150-159; Yang, J. H., Johnson, A. C., Colakoglu, S., Huang, C. A., and Mathes D. W. 2021. “Clinical and Preclinical Tolerance Protocols for Vascularized Composite Allograft Transplantation,” Online, Archives of Plastic Surgery 48(6):703-713; Colakoglu, S., Tebockhorst. S., and Mathes, D. W. 2020. “Is Vascularized Composite Allograft Transplantation Experimental of an Accepted Surgical Procedure: Results from a National Survey,” Journal of Reconstructive Microsurgery 36(4):276-280; Nakamura, Y., Takanari, K., Ebisawa. K., Kanabe, M., Nakamura, R., and Kamei, Y. 2020. “Repair of Temporal Branch of the Facial Nerve with Novel Polylycolic Acid-collagen Tube: A Case Report of Two Cases,” Nagoya Journal of Medical Science 82(1):123-128; Ng. Z. Y., Cetrulo, C. L, Jr., Lhuaire, M. Lantieri, L. A., and Lellouch, A. G. 2020. “Discussion: Full Facial Allotransplantation Including the Temporomandibular Joints: A Radiologic and Anatomical Cadaveric Study,” Plastic and Reconstructive Surgery 146(3):635-636; Dietrich, C., Nehrdich, S., Zimmer, A., Ritter, A., Hofmann, G. O., Miltner, W. H. R., and Weiss, T. 2018. “Referred Cramping Phantom Hand Pain Elicited in the Face and Eliminated by Peripheral Nerve Block,” Experimental Brain Research 236(6):1815-1824; Jensson, D., Enghag, S., Bylund, N., Jonsson, L., Wikstrom, J., and 3 others 2018. “Cranial Nerve Coactivation and Implication for Nerve Transfers to the Facial Nerve,” Plastic and Reconstructive Surgery 141(4):582e-585e; Tasigiorgos, S., Kollar, B., Krezdorn, N., Bueno, E. M., Tullius, S. G., and Pomahac, B. 2018. “Face Transplantation-Current Status and Future Developments,” Online, Transplant International 31(7):677-688; Topcu, C., Uysal, H., Ozkan, O., Ozkan, O., Polat, O. and 5 others 2018. “Recovery of Facial Expressions Using Functional Electrical Stimulation after Full-face Transplantation,” Online, Journal of NeuroEngineering and Rehabilitation 2018:15:15; De Letter, M., Vanhoutte, S., Aerts, A., Santens, P., Vermeersch, H., and 4 others 2017. “Journal of Plastic, Reconstructive, and Aesthetic Surgery 70(6):729-733; Aycart, M. A., Perry, B., Alhefzi, M., Bueno, E. M., Kueckelhaus. M., Fischer, S., and Pomahac, B. 2016. “Surgical Optimization of Motor Recovery in Face Transplantation,” Journal of Craniofacial Surgery 27(2):286-292; Kueckelhaus, M., Fischer, S., Seyda, M., Bueno, E. M., Aycart, M. A. and 4 others 2016. “Vascularized Composite Allotransplantation: Current Standards and Novel Approaches to Prevent Acute Rejection and Chronic Allograft Deterioration,” Online, Transplant International 29(6):655-662; Uysal. H., Oxkan, O., Barcin, E., Senol, U., Tombak, K., and Ozkan, O. 2016. “Referred Facial Sensation on the Hand after Full-face Transplantation,” Neurology 86(9):836-839; Cetrulo, C. L. Jr., Drijkoningen, T. and Sachs, D. H. 2015. “Tolerance Induction via Mixed Chimerism in Vascularized Composite Allotransplantation: Is It Time for Clinical Application?,” Current Opinion in Organ Transplantation 20(6):602-607; Jindal, R., Unadkat. J., Zhang, W., Zhang, D., Ng. T. W., and 7 others 2015. “Spontaneous Resolution of Acute Rejection and Tolerance Induction with IL-2 Fusion Protein in Vascularized Composite Allotransplantation,” Online, American Journal of Transplantation (New York, New York) 15(5):1231-1240; Audolfsson, T., Rodriguez-Lorenzo, A., Wong. C., Cheng, A. Kildal, M., Nowinski, D., and Rozen, S. 2013. “Nerve Transfers for Facial Transplantation: A Cadaveric Study for Motor and Sensory Restoration,” Plastic and Reconstructive Surgery 131(6):1231-1240; Weissenbacher, A., Hautz, T. Pratschke, J., and Schneeberger, S. 2013. “Vascularized Composite Allografts and Solid Organ Transplants: Similarities and Differences,” Current Opinion in Organ Transplantation (Hagerstown, Maryland) 18(6):640-644; Falcioni, M., Taibah, A., Russo, A. Piccirillo, E., and Sanna, M. 2003. “Facial Nerve Grafting,” Otology and Neurotology 24(3):486-489).

In any compound vascular bypass transplant, corresponding vessels of the donor, having been maintained past death on life support and the recipient, are connected to one another. Long-to-short switchable bloodlines as addressed in section 2b(1)(b)(b13) to follow, entitled Bloodline and Drugline Switch Devices: Long when Intercorporeal to Short when Intracorporeal to Accommodate Growth, to follow pertinent to situations where the shorter bloodline is to be implantable, are not used in a facial transplant where space to accommodate the smaller of the two lines is unavailable. Composite graft pedicles may include sensory and motor nerves or these may require to be separately dissected. For a full-face graft, the main pedicles are bilateral.

Instead, as shown at the top and bottom of FIGS. 22A and 22B, to span the distance between the two, connection is at first by means of interposing a length of synthetic tubing as bloodline between the vascular servovalves attached to the corresponding vessels, which in a face transplant includes, for example, the facial arteries and veins of the donor and recipient (see, for example, Parker, A., Bachar. C., Rodriguez-Colon, R., Hao Y., Kurian, K., and 4 others 2022. “Recipient Selection Criteria for Facial Transplantation—A Systematic Review,” Annals of Plastic Surgery 89(1):105-112; Vyas, K., Bakri, K., Gibreel, W., Cotofana, S., Amer, H., and Mardini, S. 2022. “Facial Transplantation,” Facial Plastic Surgery Clinics of North America 30(2):255-269; Belanger, M W., Borsuk, D. E., Clairoux, A. Fortier, L. P., Nguyen, A, and 5 others 2021. “Face Transplantation: Anesthesia and Other Organizational Consideration,” Canadian Journal of Anaesthesiology 68(6):868-875; Diep. G. K., Berman, Z. P., Alfonso, A. R., Ramly, E. P., and 5 others 2021. “The 2020 Facial Transplantation Update: A 15-year Compendium,” Plastic and Reconstructive Surgery Global Open 9(5):e3586; Kantar, R. S., Alfonso, A. R., Diep. G. K., Berman, Z. P., Rifkin, W. J., and 5 others 2021. “Facial Transplantation: Priciples and Evolving Concepts,” Plastic and Reconstructive Surgery 147(6):1022e-1038e; Bettoni, J., Baledent, O., Petruzzo, P., Duisit, J., Kanitakis, J., and 5 others 2020. “Role of Flow Magnetic Resonance Imaging I the Monitoring of Facial Allotransplantations: Preliminary Results on Graft Vasculopathy,” International Journal of Oral and Maxillofacial Surgery 49(2):169-175; Goel, P., Munabi, N., Nagengast, E. S., Williams, M. S., Fahradyan, A., and Urata, M. M. 2020. “The Monobloc with Facial Bipartition: Outcomes of Simultaneous Multidimensional Facial Movement Compared with Monobloc Distraction of Facial Bipartition Alone,” Annals of Plastic Surgery 84(5S): S288-S294; Kania, K., Chang, D. K., Abu-Ghname, A. Reece, E. M., Chu, C. K., and 3 others 2020. “Microsurgery Training in Plastic Surgery,” Plastic and Reconstructive Surgery Global Open 8(7):e2898; Shokri. T., Saadi. R., Wang, W., Reddy, L., and Ducic, Y. 2020. “Facial Transplantation: Complications, Outcomes, and Long-term Management Strategies,” Seminars in Plastic Surgery 34(4):245-253; Sweeney, N. G., Bernstein, G. L., and Rodriguez, E. D. 2019. “Facial Transplantation for an Irreparable Central and Lower Face Injury: A Modernized Approach to a Classic Challenge,” Plastic and Reconstructive Surgery 144(2):264e-283e; Farber, S., Kantar, R. S., Diaz-Siso, J. R., and Rodrigues, E. D. 2018. “Face Transplantation: An Update for the United States Trauma System,” Journal of Craniofacial Surgery 29(4):832-838; Kollar. B. and Pomahac. B., 2018. “Facial Restoration by Transplantation,” The Surgeon 16(4):245-249; Rifkin, W. J. David, J. A., Plana, N. M., Kantar, R. S., Diaz-Siso, J. R., and 3 others 2018. “Achievements and Challenges in Facial Transplantation Annals of Surgery (Philadelphia, Pennsylvania) 268(2):260-270; Tasigiorgos, S. 2018. “Kollar, B., Krezdorn, N., Bueno, E. M., Tullus, S. G., and Pomahac, B. 2018. “Face Transplantation-Current Status and Future Developments,” Transplant International 31(7):677-688; Russo, J. E. and Genden, E. M. 2016. “Facial Transplantation,” Facial Plastic Surgery Clinics of North America 24(3):367-377; Sosin, M., Ceradini, D. J., Hazen, A., Levine, J. P., Staffenberg, D. A., and 5 others 2016. “Total Face, Eyelids, Ears, Scalp, and Skeletal Subunit Transplant Cadaver Simulation: The Culmination of Aesthetic, Craniofacial, and Microsurgery Principles,” Plastic and Reconstructive Surgery 137(5):1569-1581; Eun, S.-C., 2015. “Facial Transplantation Surgery Introduction,” Online, Journal of Korean Medical Science 30(6):669-672; Alam, D. S. and Chi, J. 2013, “Facial Transplantation for Massive Traumatic Injuries,” Otolaryngology Clinics of North America 46(5):883-901; Pomahac, B., Pribaz, J. J., Bueno, E. M., Sisk, G. C., Diaz-Siso, Jr., and 4 others 2012. “Novel Surgical Technique for Full-face Transplantation,” Journal of the American Society of Plastic Surgeons. Plastic and Reconstructive Surgery 130(3):549-555; Leonard, D. A., Gordon, C. R., Sachs, D. H., and Cetrulo, C. L. Jr. 2012. “Immunobiology of Face Transplantation,” Journal of Craniofacial Surgery 23(1):268-271; Kim, C. W., Do E. R., and Kim, H. T. 2011. “A New Facial Composite Flap Model (Panorama Facial Flap) with Sensory and Motor Nerve from Cadaver Study for Facial Transplantation,” Online, Journal of the Korean Cleft Palate-Craniofacial Association 12:86-92; Lohn, J. W., Penn, J. W., Norton, J., and Butler, P. E. 2011. “The Course and Variation of the Facial Artery and Vein: Implications for Facial Transplantation and Facial Surgery,” Annals of Plastic Surgery 67(2):184-188; Kelly, E. J. Jacoby, C., Terenghi, G., Mennen, U., Ljungberg, C. and Wilberg, 2007. “End-to-side Nerve Coaptation: A Qualitative and Quantitative Assessment in the Primate,” Journal of Plastic, Reconstructive, and Aesthetic Surgery 60(1):1-12).

The valves fully opened so that the circulation of the recipient completely bypasses his own native organ or volume of tissue, instead flowing exclusively to and through the graft, the native supply and drainage vessels of the donor are transected at a level extending beyond the valves long enough to provide the stumps needed to allow secure anastomosis to the corresponding stumps of the recipient. Blood continuing to flow through the synthetic tubes to bypass the level for prospective anastomosis, the native vessels of the recipient are transected, the recipient structure removed, the synthetic arterial and venous tubes bent to allow positioning the graft at the recipient for anastomosis, and the stumps of donor and recipient aligned and anastomosed. Circulation continues around the level of the anastomoses through the synthetic tubes and the graft without interruption.

This sequence of steps is the same whether the graft is a lung as shown in FIGS. 22A and 22B or any other solid organ or gland, or a vascularized composite volume of tissue. Critical to graft survival, durability, and the ability to lessen the dosage of potentially harmful drugs over the ensuing years, is the uninterrupted perfusion of the donor organ, hence, the complete elimination of ischemia, reperfusion injury, and surgical trauma. As shown at the top of FIG. 22B, blood flow is never interrupted but continued through the valves and synthetic tubing so that flow is circulated around and not through the anastomoses along the vessels between the valves.

Were the transplant deep, the anastomoses straddling valves with their accessory channels would be retained to allow continued medicinal support of the anastomoses by the implanted automatic disorder response system through their accessory channels. In the face where the cosmetic effect is paramount, this is permissible if exchanging the valves for simple side-entry diversion valves as shown in U.S. Pat. No. 11,759,186, entitled Ductus Side-entry Jackets and Prosthetic Disorder Response Systems, does not result in noticeable protrusion.

Accordingly, once the anastomosis between graft and recipient have fully healed, the takeoff valve jacket at a higher level along the arterial tree, mainline, or bloodline, and terminal or graft jackets are removed. If positioned deeply, removal is omitted if space permits or accomplished endoscopically. To continue the automatic release of drugs to the graft and its neighboring tissue, the graft jacket is replaced with a basic side-entry jacket. Direct targeting of an immunosuppressant and antimicrobial to the graft and neighboring tissue avoids the immunological compromise of the recipient to pathogens.

Switch transplantation with composite tissue structures such as limbs or the face achieves ischemia-free transfer of the body part. When the prospective recipient and deceased donor continued on life support past death are both present, compound vascular bypass transplantation eliminates a period of nonperfused graft organ preservation. This continuity of perfusion as the organ is transferred between their circulatory systems is as important to graft survival as is the perioperative and postprocedural ability to directly target immunosuppressants and antimicrobials to and through the graft organ.

This capability, imparted by the valve accessory channels, makes it possible to maximize immunosuppression of the graft organ while minimizing immunosuppression of the recipient, and this capability reduces the susceptibility of the patient to pathogens at his weakest. Affording immune tolerance induction integral with the switching of the donor body part, here a full-face transplant, from the circulatory system of the donor to that of the recipient, a major factor in rejection which affects fully eighty percent of these procedures, is blunted ab initio.

That combined with the concurrent administration of an immunosuppressant such as tacrolimus, mycophenolate mofetil, and gradually reduced prednisone as an anti-inflammatory, and antimicrobials such as vancomycin and cefazolin improve the prospects for success and the indicia for evaluating this are fairly well established (see, for example, Borges, T. J., Abarzua. P., Gassen, R. B., Kollar, B., Lima-Filho, M., and 9 others 2022. “T Cell-attracting CCL18 Chemokine is a Dominant Rejection Signal during Limb Transplantation,” Cell Reports Medicine 3(3):100559; Yang, J. H., Johnson A. C., Colakoglu, S., Huang. C. A., and Mathes, D. W. 2021. “Clinical and Preclinical Tolerance Protocols for Vascularized Composite Allograft Transplantation, Online, Archives of Plastic Surgery (Seoul, South Korea) 48(6):703-713; Kauke, Tchiloemba, B., Haug. V., Kollar, B., Safi, A. F., and Pomahac, B. 2020. “Partial Loss of Nasal Tissue in Facial Vascularized Composite Allograft Patient,” Online, Plastic and Reconstructive Surgery Global Open (Philadelphia, Pennsylvania) 14(8):e3038; Kollar, B., Rizzo, N. M., Borges, T. J., Haug. V., Abdulrazzak, O., and 10 others 2020. “Accelerated Chronic Skin Changes without Allograft Vasculopathy: A 10-year Outcome Report after Face Transplantation,” Surgery 167(6):991-998; Bettoni, J., Balrfrny, O., Petruzzo, P., et al 2019. “Role of Flow Magnetic Resonance Imaging in the Monitoring of Facial Allotransplantation: Preliminary Results on Graft Vasculopathy,” International Journal of Oral and Maxillofacial Surgery 49:169-175; Haug, V., Kollar, B., Obed, D., Kiwanuka, H., Turk, M., and 5 others 2019. “The Evolving Clinical Presentation of Acute Rejection in Facial Transplantation,” Journal of the American Medical Association Facial Plastic Surgery 21(4):278-285; Kollar, B., Kamat, P. Klein, H. J., Waldner, M., Schweizer. R. and Plock, J. A. 2019. “The Significance of Vascular Alterations in Acute and Chronic Rejection for Vascularized Composite Allotransplantation,” Journal of Vascular Research 56(4):163-180; Kollar, B., Uffing, A., Borges, T. J., Shubin, A. V., Aoyama, B. T., and 8 others 2019. “MMP3 [matrix metalloproteinase-3] is a Non-invasive Biomarker of Rejection in Skin-bearing Vascularized Composite Allotransplantation: A Multicenter Valication Study,” Online, Frontiers in Immunology (Lausanne, Switzerland) 10:2771; Kollar, B., Shubin, A., Borges, T. J., Tasigiorgos, S., Win. T. S., and 8 others 2018. “Increased Levels of Circulating MMP3 [matrix metalloproteinase-3] Correlate with Severe Rejection in Face Transplantation,” Online, (Nature Research) Scientific Reports 8(1):14915; Krezdom, N., Lian, C. G., Wells, M., Wo, L., Tasigiorgos, S., and 7 others 2019. “Chronic Rejection of Human Face Allografts,” American Journal of Transplantation 19(4):1168-1177; Lee, Z. H., Lopez, C. D., Plana, N. M., Caplan, A. L., and Rodriguez, E. D. 2019. “Are We Prepared for the Inevitable? A Survey on Defining and Managing Failure in Face Transplantation,” Plastic and Reconstructive Surgery. Global Open 7(5):e2055; Kollar, B., Shubin, A., Borges, T. J., Tasigiorgos, S., Win. T. S., and 8 others 2018. “Increased Levels of Circulating MMP3 [matrix metalloproteinase 3] Correlate with Severe Rejection in Face Transplantation,” (Nature Research) Scientific Reports 8(1):14915; Tasigiorgos, S., Kollar, B., Krezdorn, N., Bueno, E. M., Tullius, S. G., and Pomahac, B. 2018. “Face Transplantation-Current Status and Future Developments,” Transplant International 31(7):677-688; Win, T. S., Murakami, N., Borges, T. J., Chandraker, A., Murphy, G., and 10 others 2017. “Longitudinal Immunological Characterization of the First Presensitized Recipient of a Face Transplant,” Journal of Clinical Investigation Insight 2(13). pii: 93894; Chandraker, A., Arscott, R., Murphy, G., Lian, C., Bueno, E., and 5 others 2016. “Face Transplantation in a Highly Sensitized Recipient,” Military Medicine (Washington, D. C.) 181(5 Supplement): 221-226; Heyes. R., Iarocci, A., Tchoukalova, Y., and Lott, D. G. 2016, Op cit. above in section 2b(1)(b)(b11), entitled Metered Compound Vascular Bypass, or Switched-organ, Heterotopic Intact Allograft Thyroid and Parathyroid Transplantation; Kueckelhaus, M., Fischer, S., Seyda, M., Bueno, E. M., Aycart, M. A., and 4 others 2016. “Vascularized Composite Allotransplantation: Current Standards and Novel Approaches to Prevent Acute Rejection and Chronic Allograft Deterioration,” Transplant International 29(6):655-662; Ashvetiya, T., Mundinger, G. S., Kukuruga, D., Bojovic, B., Christy, M. R., Dorafshar, A. H., and Rodriguez, E. D. 2014. “Donor-recipient Human Leukocyte Antigen Matching Practices in Vascularized Composite Tissue Allotransplantation: A Survey of Major Transplantation Centers,” Plastic and Reconstructive Surgery 134(1):121-129; Chandraker, A., Arscott, R., Murphy, G. F., Lian, C. G., Bueno, E. M., and 5 others 2014. “The Management of Antibody-mediated Rejection in the First Presensitized Recipient of a Full-face Allotransplant,” American Journal of Transplantation 14(6):1446-1452; Eun, S. C. 2014. “Facial Transplantation Surgery,” Archives of Plastic Surgery 41(2):174-180; Pomahac, B., Pribaz, J., Eriksson, E., Bueno, E. M., Diaz-Siso, J. R., and 15 others 2012. “Three Patients with Full Facial Transplantation,” New England Journal of Medicine 366(8):715-722).

The metered switch method should allow material improvement in full-face transplantation. Results using the conventional procedure have so far proven disappointing, the more so as the face, as key factor in individual identity and expression as perceived by others, must not be rejected after a few years to require replacement. Unless the robustness of a new face can be improved so that even in a young child, the graft remains to the end of life, a better approach is to provide a high quality prosthesis with sufficient pliancy to avoid an obvious static or plastic appearance and which can be enlarged with no change in facial features other than those associated with normal growth.

Compared to conventional microsurgery where ideally all 24 local, relatively superficial, vessels (17 arteries and 7 veins) supplying and draining the face can each be separately reconnected, switch technique requires gaining access to these angiosomes or surface blood supply territories centrally enough to materially reduce the number of diversion valves required. When the donor has been sustained past death with endocrine support, warmth, nutrition, and with no interruption in circulation or respiration as described in section 2a, reciprocal cross-circulation between donor and recipient without interruption in blood flow requires dissection beneath the donor graft to access the vessels required.

The diversion valve-jackets with mainlines are connected to diversion valve-jackets on the vessels in the recipient of corresponding caliber. Reciprocal cross-circulation is therefore not through pedicled perforator flaps that lead to the common supply and drainage vessels in the donor. For continuity of reciprocal cross-circulation throughout the transplantation procedure, connection to nearby corresponding vessels in the recipient can avert the need for disconnection for reconnection to vessels that should have been used in the first place. Which vessels and how proximate these are depends upon the extent and depth of recipient tissue destroyed due to trauma, such as avulsion or burning, or resected due to disease, such as carcinoma or necrotizing fasciitis, for example.

To allow a measure of immune tolerance induction concomitant to transfer of the face from the donor to the recipient, transplantation is by the metered switch method described above in section 2b(1)(b), entitled Metered Compound Vascular Bypass, or Metered Switched-organ Transplantation. A major benefit of the switch method for facial transplantation, and in cases of deep injury to be repaired by replantation pertains to preservation of the graft prior to replantation or transplantation. Conventionally, a face goes without perfusion from the time it is avulsed or severely injured. The face or severed portion thereof is brought to the clinic in a plastic bag where it is placed in cold storage. Only large centers will have facial reconstructive microsurgeons present and prepared to undertake the extensive anastomoses required by conventional replantation or transplantation upon arrival of the patient.

If not, the face is preserved in cold storage with the numerous and basic anoxia-reperfusion multilevel injury this entails. Compared to the detailed microsurgery prescribed by convention, initiating reciprocal cross-circulation between larger, more central, corresponding vessels in donor and recipient can be undertaken by less highly specialized operators, thus eliminating zero ischemic time with the degradation in the graft that this causes at every level from molecular to gross anatomical even before the issue of reperfusion injury arises. The graft best preserved, highly trained specialists need not be present from the outset.

Because replantation usually involves the reattachment of a face avulsed by machinery or an assault rather than a crushing or burning injury, the graft is relatively superficial and thin with only the terminal portions of the angiosomal vessels (and nerves, and muscle attachments) within it, and providing no more central vessels to allow these to be accessed and perfused together. In this circumstance, anastomosing the vessel termini respective of the angiosomes to create a more central common vessel rather than immediately anastomosing each of these to the corresponding vessels of the patient can be accomplished in less or equal time to allow a prompter initiation of continuous graft perfusion.

Accordingly, using the switch method, transplantation of a graft with higher caliber vessels is less difficult than is replantation. If the original face had been so disfigured as to preclude restoration to an identifiable condition or has been left without perfusion over a significant interval, it is preferable to transplant a more suitable graft sustained as specified in section 2a. Because the muscles of facial expression which impart animation and personality are missing or disfigured, perfection in transplantation will remain the ultimate objective.

Amplification by electrical stimulatory support of the facial muscles following transplantation by the implanted automatic system is readily accomplished (see, for example, Fischer, S., Diehm, Y., Dorante, M. I., Kotsougiani. D., Kueckelhaus, M., and 3 others 2019. “Software-based Video Analysis of Functional Outcomes of Face Transplantation,” Microsurgery 39(1):53-61; Hadιmioğlu N., Cengiz, M., Ramazanoğlu, A., Özkan, O., Ertosun, M. G., Bilal, N., and Özkan, O. 2019. “Anaesthetic Management for Face Transplantations: The Experience of Akdeniz University,” Turkish Journal of Anesthesiology and Reanimation 47(3):228-334; Jowett, N., Kearney, R. E., Knox, C. J., and Hadlock, T. A. 2019. “Toward the Bionic Face: A Novel Neuroprosthetic Device Paradigm for Facial Reanimation Consisting of Neural Blockade and Functional Electrical Stimulation,” Plastic and Reconstructive Surgery 143(1):62e-76e; Bedeloglu, M., Topcu, ç., Akgul, A., Doger, E. N., Sever, R., and 5 others 2018. “Image-based Analysis of Emotional Facial Expressions in Full-face Transplants,” Journal of Medical Systems 42(3):42; Bendella, H., Rink, S., Grosheva, M., Sarikcioglu, L., Gordon, T., and Angelov, D. N. 2018. “Putative Roles of Soluble Trophic Factors in Facial Nerve Regeneration, Target Reinnervation, and Recovery of Vibrissal Whisking,” Experimental Neurology 300:100-110; Mendenhall, S. D., Ginnetti, M. T., Sawyer, J. D., Verhulst, S., West. B. L., Levin, L. S., and Neumeister, M. W. 2018. “Prevalence and Distribution of Potential Vascularized Composite Allotransplant Donors, Implications for Optimizing the Donor-recipient Match,” Plastic and Reconstructive Surgery. Global Open 6(6):e1833; Mendez, A., Hopkins, A., Biron, V. L., Seikaly, H., Zhu, L. F., and Côthe, D. W. J. 2018. Op cit. above in section 2b(a); Rajput, M. S. A. Ahmad, A. N., Arain, A. A., Adeel, M., Akram, S., Awan, M. S., and Bari, M. E. 2018. “Preservation of Hearing and Facial Nerve Function with the Microsurgical Excision of Large Vestibular Schwannomas: Experience with the Retrosigmoid Approach,” Online, Cureus (Palo Alto, California) 10(12):e3684; Topçu, C., Uysal. H., Özkan, O., Özkan, O., Polat, O., and 5 others 2018. “Recovery of Facial Expressions Using Functional Electrical Stimulation after Full-face Transplantation,” Journal of Neuroengineering and Rehabilitation 15(1):15; Bosurgi, L., Hughes, L. D., Rothlin, C. V., and Ghosh, S. 2017. “Death Begets a New Beginning,” Immunological Reviews 280(1):8-25; Daniel. R. T., Tuleasca. C., George, M., Pralong, E., Schiappacasse, L., and 3 others 2017. “Preserving Normal Facial Nerve Function and Improving Hearing Outcome in Large Vestibular Schwannomas with a Combined Approach: Planned Subtotal Resection followed by Gamma Knife Radiosurgery,” Acta Neurochirurgica (Vienna, Austria) 159(7):1197-1211; Siemionow, M. 2017. “The Decade of Face Transplant Outcomes,” Journal of Materials Science. Materials in Medicine 28(5):64; Topçu, C., Uysal. H., Özkan, O., Özkan, O., Polat, O., 7 others 2017. “Assessment of Emotional Expressions after Full-face Transplantation,” Neural Plasticity 2017:8789724; Wegrzyn, M., Vogt. M., Kireclioglu, B., Schneider, J., and Kissler, J. 2017. “Mapping the Emotional Face. How Individual Face Parts Contribute to Successful Emotion Recognition,” Online, PLoS [Public Library of Science] One 12(5):e0177239; Aycart. M. A., Perry, B., Alhefzi, M., Bueno, E. M., Kueckelhaus, M., Fischer, S., and Pomahac, B. 2016. “Surgical Optimization of Motor Recovery in Face Transplantation,” Journal of Craniofacial Surgery 27(2):286-292; Dalal, A. 2016. “Face Transplantation: Anesthetic Challenges,” Online, World Journal of Transplantation (Pleasanton, California) 6(4):646-649; Eun, S. C. 2015. “Facial Transplantation Surgery Introduction,” Journal of Korean Medical Science 30(6):669-672; Janis, J. E., Khansa, I., Lehrman, C. R., Orgill, D. P., and Pomahac. B. 2015. “Reconstructive Management of Devastating Electrical Injuries to the Face,” Plastic and Reconstructive Surgery 136(4):839-847; Eun, S. C. 2014, Op cit. this section; Kleiss, I. J., Knox, C. J., Malo, J. S., Marres, H. A., Hadlock, T. A., and Heaton, J. T. 2014. “Whisking Recovery after Automated Mechanical Stimulation during Facial Nerve Regeneration,” Journal of the American Medical Association Facial Plastic Surgery 16(2):133-139; Miyata, H., Nishimura, R., Okanoya, K., and Kawai, N. 2012. “The Mysterious Noh Mask: Contribution of Multiple Facial Parts to the Recognition of Emotional Expressions,” Pubic Library of Science One 7(11):e50280; Shanmugarajah, K., Hettiaratchy, S., and Butler, P. E. 2012. “Facial Transplantation,” Current Opinion in Otolaryngology and Head and Neck Surgery 20(4):291-297; Bernat, I., Grayeli, A. B., Esquia, G., Zhang, Z., Kalamarides, M., and Sterkers. O. 2010. “Intraoperative Electromyography and Surgical Observations as Predictive Factors of Facial Nerve Outcome in Vestibular Schwannoma Surgery,” Otology and Neurotology 31(2):306-312; Hivelin, M., Siemionow. M., Grimbert. P., and Lantieri, L. 2009. “Extracorporeal Photopheresis: From Solid Organs to Face Transplantation,” Transplant Immunology 21(3):117-128; Yang, I., Sughrue, M. E., Han, S., Fang, S., Aranda, D., and 3 others 2009. “Facial Nerve Preservation after Vestibular Schwannoma Gamma Knife Radiosurgery,” Journal of Neurooncology 93(1):41-48; Nemeth, E. and Cigic. T. 1999. “Recovery of Facial Nerve Function Using Sural Nerve Transplantation after Its Injury in Acoustic Nerve Surgery,” (in Croatian with English abstract at Pubmed), Medicinski Pregled [Medical Review] (Novi Sad, Serbia) 52(6-8):283-286).

Ischemia and infection are no less responsible for graft failure than is immune rejection. Switch transplantation eliminates ischemia, reduces the risk of infection, and metered compound vascular bypass, or sudden switched-organ transplantation also reduces the odds for an adverse immune rejection. Improved methods for accomplishing composite tissue transplants should produce more successful results and achieve better cost effectiveness (see, for example, Lambrinos, A., Xie, X., Higgins, C., and Holubowich, C. 2016. “Composite Tissue Transplant of Hand or Arm: A Health Technology Assessment,” Ontario Health Technology Assessment Series 16(13):1-70. eCollection; Broyles, J. M., Alrakan, M., Ensor, C. R., Khalifian, S., Kotton, C. N., and 4 others 2014. “Characterization, Prophylaxis, and Treatment of Infectious Complications in Craniomaxillofacial and Upper Extremity Allotransplantation: A Multicenter Perspective,” Plastic and Reconstructive Surgery 133(4):543e-551e). The scarcity of donors, especially pediatric, mandates that these transplants succeed and hold for many years (see, for example, Mendenhall, S. D., Sawyer, J. D., West. B. L., Neumeister, M. W., Shaked, A., and Levin, L. S. 2019. “Pediatric Vascularized Composite Allotransplantation: What is the Landscape for Obtaining Appropriate Donors in the United States?,” Pediatric Transplantation 23(5):e13466).

In a prescription compliance-undependable patient, the implanted response system automatically transmits medication directly to the graft. For this reason, an important advantage in the use of an implanted automatic response system is the automated directly pipeline targeted release of immunosuppressant, anti-inflammatory, and antimicrobial medication, especially significant in the very young and very old not likely to adhere to a prescription. This targeting neither conflicts with nor imposes restrictions upon the use of systemic medication oral, injected, or infused.

Because body parts of a vascularized composite tissue allotransplantation are not essential for survival, the replacement of these is a nonemergency procedure, allowing time to transport the prospective recipient to the center where the donor expired and time can be taken to perform the transplant using the metered switch method. As delineated above in section 2b(1)(b), use of the metered technique affords a measure of immune tolerance induction as the graft is gradually transferred from the circulatory system of the donor to that of the recipient. Ischemia-free transfer requires that the recipient be positioned beside the deceased donor on life support or central cardiorespiratory stimulatory sustainment, as addressed above in section 2a. Unless wireless, wires to power the valves are run alongside the sidelines to the accessory channels.

The mainlines are removed before anastomosis; the valves and sidelines remain to target drugs to the graft. The transplantation reciprocal cross-circulation mainline valve-jackets in the donor are positioned within the graft just short of the prospective anastomosis or junction, which must be flush. The vessels there small with space for the valves wanting, this portion of the operation is microsurgical. In contrast, the mainline valve-jackets in the recipient are positioned centrally along the arterial and venous trees where the vessels are large and easily valve-jacketed. For example, in replacing the antebrachium, or forearm, these jackets can be placed along the subclavian artery and vein. In general, to avoid the need to valve jacket multiple branches, valve-jackets are positioned at the most central, or highest, level available.

Donor and recipient are positioned parallel to one another, and for connection of the recipient to the donor valves, are moved as closely together as possible. The valve mainlines, or bloodlines, of like caliber as the input vessels in the graft organ, are brought out and tunneled subcutaneously through the upper arm and down to the level of the anastomosis, where these exit, continue through expansion fabric-based lines through the intervening space to allow placement of the valves in the graft organ. The drug reservoirs, with outlet or outlets facing the adjacent axilla, are positioned subcutaneously beneath the clavicle. The sideline or sidelines of the graft accessory channels are tunneled to subcutaneously pass laterally just inside the bottom of the axilla and down through the upper arm, or brachium.

Superior to the pectoral region where the drug reservoirs and microcontroller are implanted subcutaneously, the central, here subclavian artery, valve-jackets are blood takeoffs not requiring accessory channels or sidelines. Instead, the sidelines originate at the drug reservoir outlet motors, then run alongside the mainline to the graft valves. To avoid the need to valve jacket multiple vascular branches in the antebrachium, the level of graft transection and valve-jackets is set above the elbow, thus allowing, for example, the placement of a valve jacket on the brachial artery above the capitellum before it divides into the radial and ulnar arteries. If the anastomosis is below the elbow, two mainlines are brought down from the subclavian artery valve jacket.

In a full-face transplant, drugs that should be kept from passing the blood-brain barrier are passed through valve jacket accessory channels at a lower, less central, level that allows the branch or branches to the graft and neighboring tissue to be selected. The small branches of the carotids and jugulars can be kept patent through the occasional automatic targeting of an anticoagulant such as a subneurotoxic level of a heparin and anti-inflammatory such as prednisolone through the valve accessory channels positioned at a higher level, typically in the pectoral region, or more centrally, along the arterial tree.

Whether a given immunosuppressant, anti-inflammatory, or antimicrobial drug is best applied topically must be determined on a drug-by-drug basis (see for example, Vendel. E., Rottschäfer, V., anf de Lange. E. C. M. 2019. “The Need for Mathematical Modelling of Spatial Drug Distribution within the Brain,” Fluids and Barriers of the Central Nervous System 16(1):12; Vendel. E., Rottschäfer. V., and de Lange, E. C. M. 2019. “Improving the Prediction of Local Drug Distribution Profiles in the Brain with a New 2D Mathematical Model,” Bulletin of Mathematical Biology 81(9):3477-3507; Zhang, W., Egashira, N., and Masuda, S. 2019. “Recent Topics on The Mechanisms of Immunosuppressant Therapy-related Neurotoxicities,” Online, MDPI [Multidisciplinary Digital Publishing Institute] International Journal of Molecular Sciences (Basel, Switzerland) 20(13). pii: E3210; Bellwon, P., Culot, M., Wilmes, A., Schmidt, T., Zurich, M. G., and 8 others 2015. “Cyclosporine A Kinetics in Brain Cell Cultures and Its Potential of Crossing the Blood-brain Barrier,” Toxicology in Vitro 30(1 Part A): 166-175; Chen, M., Kumar, S., Anselmo, A. C., Gupta, V., Slee, D. H., Muraski, J. A., and Mitragotri, S. 2015. “Topical Delivery of Cyclosporine A into the Skin Using SPACE-peptide [C₄₀H₆₃N₁₅O₁₇S₂] Carriers,” Journal of Controlled Release 199:190-197; Panicker, V. V., Mathew. A., and Dhamramaratnam, A. 2012. “Cosmetically Disfiguring Side Effects of Cyclosporine,” International Journal of Trichology 4(1):50; Banks, W. A. 2009. “Characteristics of Compounds that Cross the Blood-brain Barrier,” Online, BioMed Central Neurology 9 Supplement 1: S3; Gaillard, P. J. and de Boer, A. G. 2000. “Relationship between Permeability Status of the Blood-brain Barrier and in Vitro Permeability Coefficient of a Drug,” European Journal of Pharmaceutical Sciences 12(2):95-102; Leveugle, B., Ding. W., Laurence, F., Dehouck, M. P., Scanameo, A., Cecchelli. R., and Fillit, H. 1998. “Heparin Oligosaccharides that Pass the Blood-brain Barrier Inhibit Beta-amyloid Precursor Protein Secretion and Heparin Binding to Beta-amyloid Peptide,” Journal of Neurochemistry 70(2):736-744).

Conventional methods for the preservation of donor organs and body parts fail to sustain perfusion, and this predisposes the transplant to rejection first, before the immune response and the risk of infection (see, for example, Iske, J., Nian, Y., Maenosono, R., Maurer, M., Sauer, I. M., and Tullius, S. G. 2019. “Composite Tissue Allotransplantation: Opportunities and Challenges,” Cellular and Molecular Immunology 16(4):343-349; Kaufman, C. L., Bhutiani, N., Ramirez, A., Tien, H. Y., Palazzo, M. D., and 4 others 2019. “Current Status of Vascularized Composite Allotransplantation,” American Surgeon 85(6):631-637; Kubiak, C. A., Etra, J. W., Brandacher, G., Kemp, S. W. P., Kung. T. A., Lee, W. P. A., and Cederna, P. S. 2019. “Prosthetic Rehabilitation and Vascularized Composite Allotransplantation following Upper Limb Loss,” Plastic and Reconstructive Surgery 143(6):1688-1701; Taylor, M. J., Weegman, B. P., Baicu, S. C., and Giwa, S. E. 2019.

“New Approaches to Cryopreservation of Cells, Tissues, and Organs,” Transfusion Medicine and Hemotherapy 46(3):197-215; Burlage, L. C., Tessier, S. N., Etra, J. W., Uygun, K., and Brandacher, G. 2018. “Advances in Machine Perfusion, Organ Preservation, and Cryobiology: Potential Impact on Vascularized Composite Allotransplantation,” Current Opinion in Organ Transplantation 23(5):561-567; Kueckelhaus, M., Puscz, F., Dermietzel, A., Dadras. M., Fischer, S., and 3 others 2018. “Extracorporeal Perfusion in Vascularized Composite Allotransplantation: Current Concepts and Future Prospects,” Annals of Plastic Surgery 80(6):669-678; Amin, K. R., Wong, J. K. F., and Fildes, J. E. 2017. “Strategies to Reduce Ischemia Reperfusion Injury in Vascularized Composite Allotransplantation of the Limb,” Journal of Hand Surgery [American] 42(12):1019-1024; Kueckelhaus, M., Fischer, S., Seyda, M., Bueno, E. M., Aycart, M. A., and 4 others 2016, Op cit., this section; Thaunat. O., Badet, L., Dubois, V., Kanitakis, J., Petruzzo, P., and Morelon, E. 2015. “Immunopathology of Rejection: Do the Rules of Solid Organ Apply to Vascularized Composite Allotransplantation?,” Current Opinion in Organ Transplantation 20(6):596-601; Elliott, R. M., Tintle, S. M., and Levin, L. S. 2014. “Upper Extremity Transplantation: Current Concepts and Challenges in an Emerging Field” Current Reviews in Musculoskeletal Medicine 7(1):83-88; Caterson, E. J., Lopez, J., Medina, M., Pomahac, B., and Tullius, S. G. 2013. “Ischemia-reperfusion Injury in Vascularized Composite Allotransplantation,” Journal of Craniofacial Surgery 24(1):51-56; Murphy, B. D., Zuker. R. M., and Borschel. G. H. 2013. “Vascularized Composite Allotransplantation: An Update on Medical and Surgical Progress and Remaining Challenges,” Journal of Plastic, Reconstructive, and Aesthetic Surgery 66(11):1449-1455).

Vascularized composite tissue allotransplantation: The axis-proximate, or adaxial, principle for extremity replacement It is worth repeating that ischemia-free compound vascular bypass allotransplantation, which eliminates ischemia-reperfusion injury, should materially improve vascularized composite tissue allotransplantation outcomes. Unlike a full-face transplantation or a vascularized composite tissue reconstruction where a part of the body must be replaced to fit into the original site, the replacement of a portion of an extremity such as an irreparably damaged hand is not restricted thus and is best moved adaxially to reduce the intricacy and smaller size of the anatomy distad, or abaxial, thereto.

That is, a severed hand is best replaced closer to the axis which is to say higher up on the forearm (see, for example, Cassidy, M. F., Doudican, N. A., Frazzette, N., Rabbani, P. S., Carucci, J. A., and 4 others 2024. “Molecular Signature Associated with Acute Rejection in Vascularized Composite Allotransplantation,” Online, Transplantation Direct (Philadelphia, Pennsylvania) 10(10):e1714; Eldaly, A. S., Avila, F. R., Torres-Guzman, R. A., Maita, K., Garcia, J. P., and 3 others 2024. “Cell-based Therapies Induce Tolerance of Vascularized Composite Allotransplants: A Systematic Review,” Online, Journal of Surgical Research (New York, New York) 300:389-401; Grosu-Bularda, A., Hodea, F. V., Zamfirescu, D., Stoian, A., Teodoreanu, R. N., Lascar, I., and Hariga, C. S. 2024. “Exploring Costimulatory Blockade-based Immunologic Strategies in Transplantation: Are They a Promising Immunomodulatory Approach for Organ and Vascularized Composite Allotransplantation?,” Online, MDPI [Multidisciplinary Digital Publishing Institute] Journal of Personalized Medicine (Basel, Switzerland)(3):322; Ren, D., Chen, J., Yu, M., Yi, C., Hu, X., Deng. J., and Guo, S. 2024. “Emerging Strategies for Tissue Engineering in Vascularized Composite Allotransplantation: A review,” Online, Journal of Tissue Engineering (London, England) 15:20417314241254508; Sun, J. A., Adil, A., Biniazan, F., and Haykal, S. 2024. “Immunogenicity and Tolerance Induction in Vascularized Composite Allotransplantation,” Online, Frontiers in Transplantation (Lausanne, Switzerland) 3:1350546; Huelsboemer, L., Kauke-Navarro, M. Reuter, S. Stoegner, V. A. Feldmann, J., and 3 others 2023. “Tolerance Induction in Vascularized Composite Allotransplantation—A Brief Review of Preclinical Models,” Online, Transplantation International (Lausanne, Switzerland) 36:10955; Kauke-Navarro, M., Noel, O. F., Knoedler, L., Knoedler, S., Panayi, A. C., and 3 others 2023. “Novel Strategies in Transplantation: Genetic Engineering and Vascularized Composite Allotransplantation,” Journal of Surgical Research (New York, New York) 291:176-186; Siemionow, M., Kulahci, Y., and Zor, F. 2023. “Novel Cell-based Strategies for Immunomodulation in Vascularized Composite Allotransplantation,” Current Opinion in Organ Transplantation (Hagerstown, Maryland) 28(6):431-439; Sun, N., Ke. L., Qing, L., Cao, Z., Luo, G., and 5 others 2023. “The Research Landscape on Vascularized Composite Allografts: A Bibliometric Analysis (2002-2021),” Online, American Journal of Translational Research (Madison, Wisconsin) 15(3):1569-1589; Ton, C., Salehi, S., Abasi, S., Aggas, J. R., Liu, R., and 3 others 2023. “Methods of Ex vivo Analysis of Tissue Status in Vascularized Composite Allografts,” Online, [BioMed Central] Journal of Translational Medicine (London, England) 21(1):609; Zhang, L., Hoyos, I. A., Zubler, C., Rieben, R., Constantinescu, M., and Olariu, R. 2023. “Challenges and Opportunities in Vascularized Composite Allotransplantation of Joints: A Systematic Literature Review,” Online, Frontiers in Immunology (Lausanne, Switzerland) 14:1179195; Anggelia, M. R., Cheng, H. Y., Lai, P. C., Hsieh, Y. H., Lin, C. H., and Lin, C. H. 2022. “Cell Therapy in Vascularized Composite Allotransplantation,” Online, Biomedical Journal (New York, New York) 45(3):454-464. Giannis. D., Moris. D. and Cendales, L. C. 2020. “Costimulation Blockade in Vascularized Composite Allotransplantation,” Online, Frontiers in Immunology (Lausanne, Switzerland) 11:544186; Ng. Z. Y. Schweizer, R., Taddeo, A., Waldner, M., Klein, H. J., Fuchs, N., and 7 others 2020. “Adipose-derived Stromal Cell Therapy Combined with a Short Course Nonmyeloablative Conditioning Promotes Long-term Graft Tolerance in Vascularized Composite Allotransplantation,” Online, American Journal of Transplantation (New York, New York) 20(5):1272-1284; Fisher, J. D., Balmert, S. C., Zhang, W., Schweizer, R., Schnider, J. T., and 12 others 2019. “Treg-inducing Microparticles Promote Donor-specific Tolerance in Experimental Vascularized Composite Allotransplantation,” Online, Proceedings of the National Academy of Sciences of the United States of America (Washington, D C) 116(51):25784-25789; Kollar, B., Pomahac, B., and Riella, L. V. 2019. “Novel Immunological and Clinical Insights in Vascularized Composite Allotransplantation,” Current Opinion in Organ Transplantation (Hagerstown, Maryland) 24(1):42-48; Lellouch, A. G., Rosales, I. A., Geoghegan, L., Gama, A. R., and 4 others 2019. “Graft Vasculopathy of Vascularized Composite Allografts in Humans: A Literature Review and Retrospective Study,” Online, Transplantation International (Lausanne, Switzerland) 32(8):831-838; Issa, F. 2016. “Vascularized Composite Allograft-specific Characteristics of Immune Responses,” Transplantation International (Lausanne, Switzerland) 29(6):672-681; Jindal, R., Unadkat, J., Zhang, W., Zhang. D., Ng. T. W. Y., and 7 others 2015. “Spontaneous Resolution of Acute Rejection and Tolerance Induction with IL-2 Fusion Protein in Vascularized Composite Allotransplantation,” American Journal of Transplantation ((New York, New York)) 15(5):1231-1240; Petruzzo, P., Gazarian, A., Kanitakis, J., Parmentier, H., Guigal. V., and 5 others 2015. “Outcomes after Bilateral Hand Allotransplantation: A Risk/Benefit Ratio Analysis,” Annals of Surgery (Philadelphia, Pennsylvania) 261:213-220; Diaz-Siso, J. R., Bueno, E. M., Sisk, G. C., Marty, F. M., Pomahac. B., and Tullius, S. G. 2013. “Vascularized Composite Tissue Allotransplantation-State of the Art,” Clinical Transplantation (Copenhagen, Denmark) 27(3):330-337).

General information pertaining to the vasculature of the face include, Siwetz, M., Widni-Pajank, H., Hammer, N., Bruneder, S., Wree. A., and Antipova, V. 2024. “Course and Relation of the Facial Vessels—An Anatomical Study,” [in English] Online, MDPI [Multidisciplinary Digital Publishing Institute] Medicina (Kaunas, Lithuania)(Basel, Switzerland) 60(5): 805; von Arx, T., Tamura, and K., Yukiya, O., Lozanoff, S. 2018. “The Face—A Vascular Perspective, A Literature Review,” Swiss Dental Journal (Bern, Switzerland) 128(5):382-392; Lee, H.-J., Won, S.- Y., Jehun, O., Hu, K.-S., Mun, S.-Y., Yang, H.-M., and Kim, H.-J. 2018. “The Facial Artery: A Comprehensive Anatomical Review,” Clinical Anatomy (New York, New York) 31(1):99-108; Vignaud, J., Hasso, A. N., Lasjaunias, P. and Clay, C. 1974. “Orbital Vascular Anatomy and Embryology,” Radiology (Easton, Pennsylvania) 111(3):617-626).

Labial arteries. In a full-face transplant, the principle favors the placement of the vascular servovalves upstream as possible so that the linguals would be accessed along the facial if not the external carotids. To present an esthetically favorable impression, if not unobtrusively positioned in deep facial fat compartments, placement of the labial and orbital arterial valves can be subcutaneously beneath the angle of the mandible (see, for example, Nguyen, J. D. and Duong, H. 2023. “Anatomy, Head and Neck: Labial Artery,” Online, Treasure Island, Florida: StatPearls Publishing Co.; Cotofana, S. Alfertshofer, M., Schenck, T. L., Bertucci, V., Beleznay, K., and 5 others 2020. “Anatomy of the Superior and Inferior Labial Arteries Revised: An Ultrasound Investigation and Implication for Lip Volumization,” Aesthetic Surgery Journal (Oxford, England) 19; 40(12):1327-1335; Papadopoulos, T. 2020. “Commentary on: Anatomy of the Superior and Inferior Labial Arteries Revised: An Ultrasound Investigation and Implication for Lip Volumization,” Aesthetic Surgery Journal (Oxford, England) 40(12):1336-1340; Samizadeh, S., Pirayesh, A., and Bertossi, D. 2019. “Anatomical Variations in the Course of Labial Arteries: A Literature Review,” Aesthetic Surgery Journal (Oxford, England) 39(11):1225-1235).

Labial veins. The same principle of most advantageous proximity and positioning of the labial venous valves to present an esthetically acceptable appearance as specified in the preceding paragraph applies to labial veins (see, for example, Siwetz, M., et al., 2024, Op cit. preceding paragraph); Rusu, M. C., Tudose, R. C., Vrapciu, A. D., Toader, C., and Popescu, S. A. 2023. “Anatomical Variations of the External Jugular Vein: A Pictorial and Critical Review,” Online, MDPI [Multidisciplinary Digital Publishing Institute] Medicina (Kaunas, Lithuania)(Basel, Switzerland) 59(3):622; Siwetz, M., Widni-Pajank, H., Hammer, N., Pilsl, U., Bruneder, S., Wree, A., and Antipova, V. 2023. “The Course and Variation of the Facial Vein in the Face-Known and Unknown Facts: An Anatomical Study,” [in English] Online, MDPI [Multidisciplinary Digital Publishing Institute] Medicina (Kaunas, Lithuania)(Basel, Switzerland) 59(8):1479; Cotofana, S., Gotkin, R. H., Frank, K., Koban, K. C, Targosinski, S., and 4 others 2019. “The Functional Anatomy of the Deep Facial Fat Compartments: A Detailed Imaging-based Investigation, Plastic and Reconstructive Surgery (Hagerstown, Maryland) 143(1):53-63; Cotofana, S., Steinke, H., Schlattau, A., Schlager, M., Sykes, J. M., and 5 others 2017. “The Anatomy of the Facial Vein: Implications for Plastic, Reconstructive, and Aesthetic Procedures,” Plastic and Reconstructive Surgery (Hagerstown, Maryland) 139(6):1346-1353).

Orbital arteries. Blood supply to the orbits, mainly derived from the ophthalmic artery with contributions from the external carotid is complex (see, for example, Bird, B. and Stawicki, S. P. 2023. “Anatomy, Head and Neck, Ophthalmic Arteries,” Online, Treasure Island, Florida: StatPearls Publishing Co.; Gupta, Neha, Motlagh, M., and Singh, G. 2023. “Anatomy, Head and Neck, Eye Arteries,” Online, Treasure Island, Florida: StatPearls Publishing Co.; Park, H. J., Lee, J. H., and Jung, W. 2022. “The Superficial Temporal Artery and Zygomatico-Orbital Artery: Superficial Arterial Distribution of the Anterior Temple Area,” Online, BioMed Research International (New York, New York) 2022:3790546; Bhogal, P., Richter, C., Schob, S., AlMatter, M., Hellstem, V., Perez, M. A., and Henkes, H. 2020. Basilar Artery Origin of an Orbital Artery-A Rare Variant and Review of the Embryology of the Orbital Arterial Supply,” Online, Interventional Neuroradiology (Thousand Oaks, California) 26(5):668-674; Bertelli, E., Regoli, M., and Bracco, S. 2017. “An Update on the Variations of the Orbital Blood Supply and Hemodynamic,” Online, Surgical and Radiologic Anatomy (Berlin, Germany) 39(5):485-496; Hayreh, S. S. 2006. “Orbital Vascular Anatomy,” [Nature Portfolio] Eye (London, England) 20(10):1130-1144). If valving at the internal carotid is not feasible, then the extracranial subcutaneous supraorbital, which anastomoses with its contralateral counterpart, the supratrochlear and superficial temporal arteries, is used (see, for example, Siwetz, M., et al., 2024, Op cit. second paragraph above).

Orbital veins. Venous drainage of the orbits is through the infraorbital, supraorbital, inferior and superior, ophthalmic veins which course through the superior and inferior orbital fissures. Anastomoses include the facial vein and pterygoid plexes (see, for example, Siwetz, M., et al., 2024, Op cit. third paragraph above)(see also Massa; R. N., Minutello; K., and Mesfin, F. B. 2023. “Neuroanatomy, Cavemous Sinus,” Online, Treasure Island, Florida: StatPearls Publishing Co.; Patel, A. K., Tripathi, A., Kumar, R., and Patel, R. B. 2021. “Anatomical and Radiological Study of Superior Orbital Fissure,” Online, Maedica (Bucharest, Romania) 16(4):599-602; De Battista, J. C., Zimmer, L. A., Theodosopoulos, P. V., Froelich, S. C., and Keller, J. T. 2012. “Anatomy of the Inferior Orbital Fissure: Implications for Endoscopic Cranial Base Surgery,” Online, Journal of Neurological Surgery. Part B. Skull Base (Stuttgart, Germany) 73(2):132-138; Isolan, G. R., Krayenbuhl, N., de Oliveira, E., and Al-Mefty, O. 2007. “Microsurgical Anatomy of the Cavernous Sinus: Measurements of the Triangles in and Around It,” Online, Skull Base (New York, New York) 17(6):357-367; Spektor, S., Piontek, E., and Umansky, F. 1997. “Orbital Venous Drainage into the Anterior Cavemous Sinus Space: Microanatomic Relationships,” Neurosurgery (Philadelphia, Pennsylvania) 40(3):532-9; discussion 539-540).

2b(1)(b)(b13). Bloodline and Drugline Switch Devices: Long when Intercorporeal to Short when Intracorporeal to Accommodate Growth

Line switches allow switching the same fluid, blood or a drug, between either of two lines. The use of expandable structure based vascular prostheses, bloodlines, druglines to allow spontaneous expansion in caliber and length to accommodate growth is essential in pediatric patients. In adults, it is not, conventional catheteric lines made for lifetime implantation suitable. While the traveling waves produced by the pulse as seen from outside the artery are suppressed while passing through the noncompliant walled catheter, once flow continues into the native artery, this action reappears. For this reason, the compliance of the expansion structure to the pulse in this regard is only advantageous to provide a visual indication of the flow of blood. For use in an adult, the operator can, however, pull expandable structure bloodline stock to the length needed.

FIGS. 32A thru 32C show a single-vessel-in/single-vessel-out switchable bloodline for exchanging a longer for a shorter bloodline, or conceivably, the reverse. Anomalous supernumerary, or accessory, vessels relatively common for kidneys, FIG. 35A shows a confluence conduit for connecting a number of input bloodlines to a different number of outlet bloodlines, of which the utility arises when the donor and recipient differ as to the number of arteries and/or veins of either in a kidney transplant, for example. FIG. 35B shows a switchable confluence conduit which likewise allows a difference in the number of inlet and outlet bloodlines to the vessels. In both drawing figures, part number 167 is a miniature inline reversible pump that allows retarding or accelerating the rate of joint input flow through the conduit.

Individual switchable lines such as shown in FIG. 35A and multiple switchable lines as shown in FIG. 35B are compatible with the use of expandable structure bloodlines as described below in sections 2b(2)(c), entitled Vascular Segment Prostheses: Inline Coupling Jackets and Bridging, or Tie-lines, and 2b(2)(f), entitled Expandable Structure to Accommodate Growth and Self-adjust in Size in Vascular Segment Prostheses, Lines, and Confluence Conduits Placed in Children. Placed in a pediatric patient early in life, expansion structure lines eliminate a progressive tightening as the child grows and the eventual need for reentry to replace these.

Equally important, expandable structure lines are compatible with automatic vessel segment to include aortic arch replacement as addressed below in section 2b(2)(d), entitled Anoxia-free Aortic Repair: Aortic Prostheses Thoracic, Thoracoabdominal, and Abdominal. Anomalous aortae more common, any vessel such as a pulmonary artery or vein can also be replaced as described below in section 2b(2)(c)(c2), entitled Automatic Insertion into a Ductus of an Inline Coupling Jacket-secured Bridging, or Tie-line and Its Replacement if Necessary.

In FIGS. 32A thru 32F, shown are catheter bloodline, or mainline, connectors that allow the initial use of bridging blood connecting mainlines much longer than when the donor and recipient during an organ transplant are positioned adjacent head to toe and allow the removal of the longer of the two lines with the flow of blood having been switched to the much shorter line to be implanted with no interruption in the flow of blood. In end stage heart failure, the impaired heart of the recipient may lack sufficient force of ejection (ejection fraction, absolute stroke volume) so that even with the assistance of medication to include one inotropic if tolerable, the heart is unable to propel blood entirely through the lines over the distance to the corresponding vessels in the donor without the assistance of an inline pump.

In such cases, longer lines or confluence conduits needed during a compound vascular organ transplant can, if necessary, be switched to a shorter version of the line or conduit where the latter incorporates the flats and vaults configuration that will allow it to grow along the patient. In FIGS. 32A thru 32F, the counterpart arterial flow has been omitted as needlessly complicating each view; if included, the venous counterpart would be identical but move blood in the opposite direction.

Also, to lend compliance with the pulse, the arterial bloodline can be encased within the expansion fabric as described below in section 2b(2)(f), entitled Expandable Structure to Accommodate Growth and Self-adjust in Size in Vascular Segment Prostheses, Lines, and Confluence Conduits Placed in Children, and shown in FIGS. 15C, 15D, 36A, and 36F, used in any fluid line or conduit for pediatric use where spontaneous enlargement to accommodate growth following implantation is essential.

To meet this need, longer bridging bloodlines can incorporate an inline pump, thus overcoming a lack of adequate ejective force to propel blood to the donor. Inline pumps can also be useful in blending the blood of donor and recipient during a metered organ transplant. To allow a clearer view of the stream passing through, any type line other than one to be implanted in a pediatric patient can be made of a plasticizer and surfactant residue-free polyethylene, polypropylene, or another polymer having clear transparency. FIG. 32A shows the initial connection between donor and recipient organs with flow passed through the longer of the two lines by the bloodline-switches.

FIG. 32B shows the connection between donor and recipient organs when the bloodline-switches have been switched to pass blood flow through the shorter bloodline, the longer of the two lines having been removed. FIG. 32C shows that blood flow having been diverted to bypass the diseased native organ of the recipient, that organ can be removed and the donor organ moved into position by transfer and rotation into the circulatory system of the recipient in preparation for anastomosis to the venous stump of the recipient following removal of the diseased organ. FIG. 32D shows the status in transplantation when the graft organ of the donor has been repositioned as indicated in FIG. 32C.

FIG. 32E shows the retraction of the diversion chutes in both inlet and outlet venous vascular valves allowing uninterrupted flow through the venous drainage line following anastomosis so that the expansion structured short line and bloodline-switches can be removed. FIG. 32F shows that with uninterrupted flow as per FIG. 32E, a permanent line can replace the expansion structured line and bloodline-switches and the vascular valves extended to pass flow through the permanent line to bypass the anastomosis. FIG. 32G provides a detailed view of a bloodline-switch.

FIGS. 33A and 33B provide a closer view of the flip-flop, or toggle stopper shown in FIGS. 32A thru 32D.

FIG. 34A shows a bloodline switch key and FIG. 34B a detailed view of the detent therein which allows simultaneous switching between long and short bloodlines to avoid any transient that would risk spasm or desynchronization due to interrupted blood flow to or from vessels that should not be clamped, to include the brain through the internal carotids or jugulars and the vessels of the heart.

To allow continued targeting of drugs through the accessory channels as needed, the anastomosis bypass line and vascular valves are not removed. The resultant ability to switch between bypass and throughflow, which the implanted automatic disorder response system with sensors to detect the biomarkers associated with various complications can actuate automatically, allows numerous diagnostic and therapeutic advantages to be activated in the ambulatory patient before the patient even becomes aware of the incident. Various advantages are addressed above in section 2 entitled Background of the Invention.

2b(1)(b)(b14). Confluence Conduits: Connection of One or More Bloodlines to Flow Through a Common Channel to the Same or a Different Number of Bloodlines Through a Shared Channel with Self-Leveling Apportionment of Outflow

Confluence conduits are addressed in the preceding section entitled Background. General. Compound vascular bypass, or switched-organ, transplantation calls for connecting corresponding arteries and veins between the donor and recipient organs. Donor and recipient are positioned in adjacent relation. FIG. 16 shows that in a heart transplant, nine vessels, hence, nine bloodlines to connect the eighteen vascular valves on corresponding donor and recipient vessels are required.

While outflow from a confluence conduit is ordinarily passive with one-way passive side-entry flow as described above in section 2b, entitled Vascular Valves and Servovalves in the Diversion of Blood Flow, in the subsection therein entitled One., Two., and Three-way Passive Side-entry Flow Diversion Jackets or Flow Directional Guides, the conduit can be entered by one-way passive side-entry flow diversion jackets or bistable plunger solenoid-driven valves, or servovalves.

In FIGS. 35A and 35B blood directional flow line 41 represents only the fraction of the blood volume tapped or drawn off through the servovalves to the left from each of the contributing vessels of the donor for continued flow into the confluence conduit, the fraction continuing straight through the donor vessels, usually the arteries in a heart transplant, omitted. This is equivalent to the entire volume of each vessel diverted into the confluence conduit when the servovalve diversion chutes are fully extended. At the outlet, the connectors to the respective outflow vessels 197′ are shown as equipped with diversion chutes 18. This option allows manual adjustments in the proportion of outflow into each of the outlet vessels.

Otherwise, the outlet connectors consist of simple side-entry jackets such as shown in FIG. 10F without valvular function or a diversion chute. In FIGS. 35A and 35B, the confluence conduits are depicted as unidirectional as these are intended to divide up the arterial inputs in a compound vascular bypass heart transplant where the arteries are numerous, the venous return lines generally one each for the superior and inferior venae cavae. This is why the return lines will usually be bloodlines 196′ rather than a confluence conduit 230′ were return from both the superior and inferior venae cavae conveyed.

With paired arterial-venous conduits in the reciprocal cross circulation circuit, the volume of outflow must substantially equal that of the inflow. If passed through a confluence conduit, the pulmonary artery, named as such by virtue of sending blood to rather than from the lungs, is connected to the venous conduit as conveying deoxygenated blood. The portal vein poses a similar ambiguity of nomenclature, but like the venae cavae is large enough as rarely if ever to be connected to a confluence conduit.

The conduit flow-through and outlet volumes should also be substantially equal to the sum volume of that of the vessels entering the confluence conduit when the inlet servovalve diversion chutes are fully extended. In a compound vascular organ transplantation, the volume of blood sent to the graft organ from the recipient to the donor and that returned from donor to the recipient should be continuously equal. Accordingly, FIGS. 35A and 35B show only one confluence conduit when there are always two, one arterial, and the other venous, and while for most applications it will suffice to provide a common drugline to the conduit, it is possible to provide any one or all of the entry valves or servovalves with a dedicated drugline.

Confluence conduits as shown in FIGS. 35A thru 35F for use in a prenatal, neonatal, and early toddler compound vascular heart transplant such as a shown in FIGS. 15C and 15D must be of the flats 233 and vaults 234 conformation. Confluence conduits have a number of bloodline connections at either end corresponding to the number of native vessels to be connected. Exceptionally, those having united entry for direct aortocaval connection to the abdominal aorta or to the inferior vena cava at the origin have sufficient gauge to convey the sum of bloodline volume for connection to each artery or vein of the graft organ as destination.

Whereas for a compound bypass sudden switch transplant, the diversion chutes are driven by a plunger solenoid, for a compound bypass metered switch transplant, the requirement for continuously variable volume of blood flow-through requires the use of vascular servovalves. A type confluence conduit specialized to expedite connection of the vessels of the donor and recipient hearts in prospective compound vascular bypass prenatal, neonatal, and early life heart transplants are shown in FIGS. 15C and 15D. The different types of valves addressed above in section 2a, entitled Background of the Invention. General, and in section. 6, entitled Description of the Preferred Embodiments of the Invention, the valves used in these would almost always be bistable plunger solenoid-driven to implement a sudden switch transplant recommended by the immature immune system of the patient and eliminating the expense of minute micromachined valves, the need for microsurgical experience, and significantly reducing both the duration and expense of the operation.

Of the vessels involved, the upper and lower venae cavae, pulmonary artery, and aorta are larger, which distinction is reflected in the diameters of the bloodlines leading into and out of the confluence conduit. If anomalous in the patient, they are included in the graft. The ischemia-free replacement of these vessels later in life due to congenital or acquired defects are accomplished as delineated below in section 2b(2)(c), entitled Vascular Segment Prostheses: Inline Coupling Jackets and Bridging, or Tie-lines, and its subordinate sections.

In a growing patient, the need for later reentry is eliminated through the use of replacement prostheses such as confluence conduits and bridges or tie-lines are made with an elastic and expandable fabric with high tensile strength for resistance to bursting or rupture, described below in section 2b(2)(f), entitled Expandable Structure to Accommodate Growth and Self-adjust in Size in Vascular Segment Prostheses, Lines, and Confluence Conduits Placed in Children. The advantage of these over conventional surgical repairs in terms of durability and freedom from later adverse sequelae is considerable and explained above in section 2a, entitled, Background of the Invention. General. This is especially important for prenates, neonates, infants, and toddlers who are spared what would otherwise be an inevitable need for at least one painful revision due to growth.

FIGS. 35A through 35F show a confluence conduit for use in an adult, which consists, for example, of a length of medical grade polyetheretherketone polymer tubing internally and externally coated to thwart the accretion of clot and biofilm which has been cut to the length suitable for the patient. To allow for growth in a pediatric patient, the confluence conduit is made with the expandable structure addressed below in section 2b(2)(f), entitled Expandable Structure to Accommodate Growth and Self-adjust in Size in Vascular Segment Prostheses, Lines, and Confluence Conduits Placed in Children, which has been cut to the appropriate length for the extent of growth remaining.

Expandable structure stock is provided in various diameters in a wound coil from which the operator or a technician cuts off a length sized for the immediate patient. The length cut is then fitted with an end-cap of the same diameter or gauge at either end. Confluence conduit end-caps match in diameter and snap on to the flat, not vault, ends of the length of conduit cut. End-caps provide punch-out holes of different sizes proportional to their diameter which are removed by pushing through the bloodlines from the simple side-entry jacket or metering vascular servovalves connected to the source input and output vessels. Retention in the end-caps of the bloodlines is by tight friction fit and cyanoacrylate cement.

Section 1a entitled Background. General, mentions that ordinarily, a confluence conduit has a has a volume delivery rate in milliliters per second equal to the sum of the volume delivery rates of the bloodlines connected to it. More efficient and better routed connections to the confluence conduit can be achieved by altering each as to the position at the entry into or along the length of the conduit. Two dimensional, however, face-on, or full-face, drawing figures as best depict the connections involved lack depth. To compensate for this third dimension for connection of bloodlines, the confluence conduits shown in FIGS. 35A thru 35F have been depicted as at least three times greater in diameter than actual.

Other vessels alike in caliber, the incorrect direct connection between donor and recipient vessels were each connected individually could result in postprocedural problems necessitating reentry, which eventuality is to be avoided. The diseased recipient heart likely associated with degradation in its arteries compared to the healthier vessels of the donor organ, whether due to elasticity, peripheral vascular resistance, or atheromatous disease, for example, different cardiac arteries differing in blood pressure blood pressure that if separately connected could result in impaired end organ function would be moderated if not eliminated first, by taking healthy vessels along with the heart from the donor, and second, interposing a confluence conduit.

Generally, it is preferable to take healthy vessels than to angioplasty the diseased vessels of the recipient. By extension, if the donor heart would likely be subject to the same intractable pulmonary vascular resistance as the native recipient heart, then a heart-lung transplantation is recommended. Portions of the bloodlines connecting corresponding vessels in the donor and recipient may be synthetic or a healthy vessel harvested along with the heart from the donor. Retaining healthy arteries preserves the advantages of the elastic laminae and smooth musculature of the natural vessels.

In a confluence conduit, addressed in this section and shown in FIG. 35A, to selectively affect the combined flow of tributaries 197 and 198 alike as distinct from any other bloodlines, drugline 8 empties into common channel or confluence conduit 230. In a switchable confluence conduit, addressed in the section to follow and shown in FIGS. 32G, 33A, 33G, and 35B, to affect the combined flow of tributaries 197 and 198 alike as distinct from any other bloodlines, drugline 8 empties into blood confluence chamber, part number 178, thus affecting the mix regardless of into which confluence conduit flow is then switched.

Confluence conduits are of special value in dealing with an unequal number of vessels supplying or draining the same part of the parenchyma in the recipient and donor organs. Commonly encountered in kidneys such vessels are referred to as accessory, supernumerary; anomalous; and when polar, aberrant, and are addressed above in section 2b(1)(b)(b8), entitled Metered Compound Vascular Bypass, or Sudden Switched-organ Single and Double Kidney Transplantation. Where the supernumerary vessel or vessels are patent and not just vestigial and neither kidney presents a condition that resists flow to increase the blood pressure, a confluence conduit can spontaneously adjust the apportionment of flow among the vessels to compensate for such disparities.

Where extracorporeal flow, such as between an ex vivo organ perfusion transport machine and the graft organ, or between such a machine and a recipient, or extracorporeal flow as ‘intercorporeal’ between an organ recipient and donor, will usually require that blood flow be regulated in volume and pressure, intracorporeal flow in a normotensive patient will ordinarily reinstate itself to normal volume and pressure. If not, then ancillary valving and medicinal means for instating normotension consistent with the use of such a prosthesis are available. An example of an intracorporeal device which does not require integral valving is an inline aortic arch prosthesis. Spontaneous flow apportionment is advantageous when transplanting kidneys with accessory or supernumerary arteries, for example, of whch the occurrence is relatively common.

### Risks Involved in the Surgical Embolization of Vessels

Renal vessel embolization, or angioembolization, is mostly done to treat renal hemorrhaging, pseudoaneurysms, angiomyolipomas, and other tumors as well as congenital vascular malformations. Both coil and injection embolization carry serious risks (see, for example, Ghouri, R. G., Ali, M. M., Naeem, H., Raza, M. S., and Arshad, W. 2024. “Anterior Spinal Artery Syndrome after Bronchial Embolization for Hemoptysis: A Case Report and Literature Review,” Online, BMJ [British Medical Journal] Neurology Open (London, England) 6(1):e000684; Jaafar, S., Zahoor, T., Bagegni, M., Alzaabi, F., Al Mazrouie, M., Ibraheem, W., and Dhou Nourein, M. 2024. “Rare Case of Massive Middle Cerebral Artery Infarct after Bronchial Artery Embolisation,” Online, European Journal of Case Reports in Internal Medicine (Milan, Italy) 11(6):004594; Ramaswamy, R. S., Akinwande, O., and Tiwari, T. 2018. “Renal Embolization: Current Recommendations and Rationale for Clinical Practice,” Current Urology Reports (Philadelphia, Pennsylvania) 19(3):5; Grenier, N., Petitpierre, F., Le Bras, Y., Lasserre, A. S., Cornelis, F. 2016. “Renal Embolization,” (in English) Néphrologie & Thérapeutique (Paris, France) Supplement 1: S139-143; Muller, A. and Rouvière, O. 2015. “Renal Artery Embolization-Indications, Technical Approaches and Outcomes,” Nature Reviews Nephrology (London, England) 11(5):288-301; Irwine, C., Kay, D., Kirsch, D., and Milbum, J. M. 2013. “Renal Artery Embolization for the Treatment of Renal Artery Pseudoaneurysm following Partial Nephrectomy,” Online, Ochsner Journal (New Orleans, Louisiana) 13(2):259-263; Ashour, R., Aziz-Sultan, M. A., Soltanolkotabi, M., Schoeneman, S. E., Alden, T. D., and 5 others 2012. “Safety and Efficacy of Onyx Embolization for Pediatric Cranial and Spinal Vascular Lesions and Tumors,” Neurosurgery (Philadelphia, Pennsylvania) 71(4):773-784; Sauk, S. and Zuckerman, D. A. 2011. “Renal Artery Embolization,” Online, Seminars in Interventional Radiology (New York, New York) 28(4):396-406; Ginat, D. T., Saad, W. E., and Turba, U. C. 2009. “Transcatheter Renal Artery Embolization: Clinical Applications and Techniques,” Techniques in Vascular and Interventional Radiology (Philadelphia, Pennsylvania) 12(4):224-239; Davis, C., Boyett, T., and Caridi, J. 2007. “Renal Artery Embolization: Application and Success in Patients with Renal Cell Carcinoma and Angiomyolipoma,” Online, Seminars in Interventional Radiology (New York, New York) 24(1):111-116; Schwartz, M. J., Smith, E. B., Trost, D. W., and Vaughan, E. D. Jr. 2007. “Renal Artery Embolization: Clinical Indications and Experience from Over 100 Cases,” BJU [British Journal of Urology] International (Oxford, England) 99(4):881-886; Bilbao, J. I., Martinez-Cuesta, A., Urtasun, F., and Cosin, O. 2006. “Complications of Embolization,” Online, Seminars in Interventional Radiology (New York, New York) 23(2):126-142; Hemingway, A. P. and Allison, D. J. 1988. “Complications of Embolization: Analysis of 410 Procedures,” Radiology (Easton, Pennsylvania) 166(3):669-672).

As addressed in the section to follow and depicted in FIGS. 35C thru 35F, long synthetic bloodlines are used to span the distance separating donor and recipient hearts only to be switched to short lines once the graft has been placed, so that the absolute time these remain in place is that of the procedure and therefore negligible. In a double heart heterotopic heart transplant, synthetic lines may continue in use where these are supported by the automatic release of a clot inhibitor by the prosthetic disorder response system. Where the anatomy allows encirclement by a valve, larger vessels are connected before branching.

Due to the large number of vessels in a heart transplant, passing all arterial flow through one confluence conduit and all venous flow through another considerably simplifies the surgical field. Otherwise, vessels exceptional in caliber, such as the large aortae and venae cavae, can be separately connected rather than included in confluence conduits, and the medium caliber pulmonary vessels can be segregated in their own arterial and venous confluence conduits. Diametric differences among the remaining vessels negligible, only one caliber need be incorporated into a second line of confluence conduits. In this way, only two size versions of confluence conduits differing only in the number of input and output bloodlines and the incorporation of bidirectional inline flow-through pumps need be produced for adults.

In a compound vascular bypass heart transplant, it should be considered that to pass all arterial flow and all venous flow through one confluence conduit each will equate to a sum conduit cross-section into which a bidirectional inline through-flow pump will less likely be needed, but if it is, the pump can be of standard dimensions and therefore of less expense. Also serving to reduce expenses is that in a switchable confluence as addressed next in section 2b(1)(b)(b15) on switchable lines and confluence conduits, the upper longer of the two conduits is removed and only that smaller implanted or not implanted on a case by case basis. To allow for growth in pediatric patients, confluence conduits are made with the highly expandable fabric described above in section 2b(2)(f), entitled Expandable Structure to Accommodate Growth and Self-adjust in Size in Vascular Segment Prostheses, Lines, and Confluence Conduits Placed in Children, as in FIG. 36F showing a prosthetic aortic neoarch.

In a metered compound vascular bypass transplant, transcatheteric or transluminal angioplasty and medication having been applied to donor and recipient, if necessary, the interposition between their corresponding supply and drainage vessels of a confluence conduit will compensate for a distinction in the number or caliber of individual vessels. When time does not permit use of the metered technique, arterial and venous confluence conduits equipped with bidirectional flow-through pumps, part number 167 in FIGS. 35A and 35B can make adjustments in blood pressure collectively responsive to the central control of blood pressure in donor and recipient rather than on a vessel-by-vessel basis. Both arterial and venous pumps can increase the conduit flow-through pressure in the antegrade or forward or reduce it in the reverse direction.

Drugs, regional anesthesia of the recipient, and the transfer procedure itself can also induce distinctions in flow between like vessels which the interposition of the conduits which flow-through pumps can moderate (see, for example, Gopalan, C. 2022. “Blood Pressure, Hypertension, and Exercise,” Chapter 8, pages 141-156 in Gopalan, C. and Kirk, E., Biology of Cardiovascular and Metabolic Diseases, Amsterdam, North Holland, Netherlands and Cambridge, Massachusetts: Elsevier/Academic Press; Persichini, R., Lai, C., Teboul, J.-L., Adda, I., Guerin, L., and Monnet, X. 2022. “Venous Return and Mean Systemic Filling Pressure: Physiology and Clinical Applications,” Critical Care (Online), 26:150; Shahoud, J. S., Sanvictores, T., and Aeddula, N. R. 2021. “Physiology, Arterial Pressure Regulation,” Online, Treasure Island, Florida: StatPearls Publishing Co.; Gelman, S., Warner, D. S., and Warner, M A. 2008. “Venous Function and Central Venous Pressure: A Physiologic Story,” Anesthesiology 108(4):735-748; Printz, M. P. and Jaworski, R. L. 2004. “Hypertension, Overview,” in Encyclopedia of Endocrine Diseases, pages 577-588, Amsterdam, North Holland, Netherlands: Elsevier/Science Direct).

If the ascending aorta curves up over and about the right pulmonary artery too closely and unyieldingly so that to separate the two enough to place the servovalve is impossible, the aorta is not valved about during its ascent. Instead, the innominate, or brachiocephalic, left common carotid, and the left subclavian arteries are connected to their counterparts separately, leaving the superior vena cava as the only large caliber vessel in the upper group. Then donor and recipient aortae are best connected when the aorta has descended below the lower margin of the heart, leaving only the head-bound smaller caliber arteries in the upper group.

Then there are twelve connections to be made so that 24 valves are needed. At a distance from their origin, the vessels of the upper group are the same in caliber, hence, barely distinguishable. An advantage in the interpositioning of a confluence conduit is that a direct donor to recipient misconnection mediated by a confluence conduit would compensate for discrepancies in flow volume and rate that might cause problems were different vessels directly. Small vessels are generally grouped separately than large vessels such as those designated as great which warrant flow through separate dedicated bloodlines.

In this way, a confluence conduit affords a factor of security in simplifying vessel connections, in that different smaller caliber vessels can be mistakenly connected without adverse consequence. FIGS. 32A thru 32F show the steps taken in the use of a long-to-short switchable bloodline used to span the distance separating the donor and recipient hearts midprocedurally, FIGS. 32G, 33A, and 33B show the type of switch used in such a bloodline, and FIG. 35B shows how the concept of the individual long-to-short switchable bloodline can be applied to a long-to-short switchable confluence conduit.

With a confluence conduit, provided larger caliber inlet and outlet bloodlines are incorporated, those larger will be immediately distinguishable from the other smaller lines, precluding their misidentification. The process of switching from a long to a shorter line using a bloodline switch is delineated in somewhat greater detail in the section to follow as a simpler case of the equivalent sequence of steps involved in the use of long-to-short or the reverse switchable confluence conduits. Where the difference in length between the long and short lines is less than considerable, a stretchable, or expandable conduit-provided to allow expansion in a vascular structure such as the thoracic aorta in a pediatric patient so that reentry can be avoided—can be made as described below in section 2b(2)(f), entitled Expandable Structure to Accommodate Growth and Self-adjust in Size in Vascular Segment Prostheses, Lines, and Confluence Conduits Placed in Children, such as the prosthetic aortic neoarch shown in FIG. 36F.

As indicated however, the other vessels substantially alike in caliber, use of a confluence conduit eliminates the adverse potential consequences of misidentifying and directly connecting different vessels. This is because the outflow into each vessel is determined not just by its caliber but also by the rate and volume of flow-through-factors that were likely affected in vessels long associated with a malfunctioning heart and not likely to be diagnosed in detail in an exigent operation to replace and end stage failing heart. The self-correction of a confluence conduit minimizes if not eliminates the adverse consequences of a direct connection between different vessels due to human error.

Accordingly, confluence conduits offer four primary advantages:

1. A confluence conduit simplifies connecting vessels between recipient and donor organs. Since a confluence conduit outputs the same volume of blood as flows into it, its use simplifies compound vascular bypass, or sudden switched-organ, transplantation of kidneys where either or both the recipient native and donor, or graft, organs present accessory, or supernumerary, arteries, for example. Provided anomalous or pathological deviations such as strictures, plaques, or significant inflammation of the recipient vessels are identified and these treated if and only if as necessary, the confluence conduit, because it provides that the overall volume of recipient output and donor input blood will be equal and level out distinctions among the vessels as to caliber. For this reason, mismatching relations of correspondence between donor and recipient vessels of like caliber should have no effect.

That is, where the donor and corresponding recipient vessels differ in number, the confluence conduit spontaneously cross-levels the volume of flow into each receiving vessel. Similarly, when the total volume of outflow of a severely malformed organ, such as a univentricular or tetralogy of Fallot heart, is normal but the apportionment of outflow among its veins which might be anomalous, is abnormal, the summarization of its outflow by interpositioning a confluence conduit between the donor and recipient veins allows, if necessary, a venous reconstruction in the recipient to allow a normal anastomosis to each on a one for one basis as if the recipient vasculature had been normal. FIGS. 15 and 20B show a one vessel into two or the reverse junction using a vascular valve when the diversion chute is fixed at the halfway extended point in its throw.

2. Connecting a combination of vessels to the same confluence conduit isolates these vessels for common therapy; an accessory channel connected to the confluence conduit as shown in FIG. 35A can be used to identically medicate the group of vessels in a targeted manner as distinct from other vessels, and the prosthetic disorder response system can continue the release of medication or change it in response to sensor feedback indefinitely. Unified entry arterial or venous entry confluence conduits are described in sections 2b(1)(b)(b14), entitled Confluence Conduits: Connection of One or More Bloodlines to Flow through a Common Channel to the Same or a Different Number of Bloodlines with Self-leveling Apportionment of Outflow, and 2b(1)(b)(b16), entitled Direct Aortocaval Connection: Unified Entry Arterial and Exit Venous Confluence Conduits for the Heterotopic Connection of a Graft Heart in the Lower Abdomen.

Druglines can be connected to any servovalve vessel leading into a confluence conduit and to treat the blood from the vessels having contributed blood to the conduit entry, druglines can also be attached anywhere along the confluence conduit. Connection thus is by means of a basic side-entry diversion jacket with fixed, or molded in, diversion chute. A typical drug would be a heparin or anticoagulant to prevent clotting, and/or thrombolytic to dissolve clots, and/or an antimicrobial, anti-inflammatory, analgesic, anesthetic, statin, chemotherapeutic, antiangiogenic, immune tolerance inducer, or an immunosuppressive.

Also, druglines can be connected to a confluence conduit anywhere along its length so that a heparin, for example, can be released into the convergence leading into the conduit, for example. Based upon an input from a sensor situated at the site or volume of tissue to which a given conduit outlet branch or line is to terminate, the master control microprocessor can command the release of the responsive medication specified in the prescription-program for delivery through a drugline into that one pertinent that line. At the same time, delivery into the body of the conduit releases medication into all of the outlet lines.

If connected to one or more outlet lines, a medicinal can be commanded released from the implanted drug reservoir. The periodic application of a statin, steroid, immunosuppressive, or anticoagulant, for example, can be administered automatically and apart from the vessels excluded from connection through the confluence conduit. Sensors and effectors such as a heating element, thermoelectric, or Peltier cooling device, and so on, can be positioned alongside the confluence conduit so as not to cause turbulent flow, and a miniature in-line flow-through pump or a servochoke can be used to selectively adjust the blood pressure.

Especially with the tiny blood vessels in an infant much less a prenate, the significant reduction in the number of vascular servovalves to one for direct aortocaval connections to the abdominal aorta and the inferior vena cava in the lower abdomen or abdominopelvic region means that confluent flow eliminates not only the servovalve per vessel that would be needed were connection consolidated but the need for additional druglines. Tiny valves on tiny vessels are more susceptible to becoming obstructed if swollen. Additionally, the need for microsurgical components and instruments when the means exist to avoid this eliminates considerable expense as well as the need for specialized expertise.

In such small vessels, combined direct aortocaval connections makes it possible to position an endovascular inline pump, part number 167 in FIGS. 35A and 35B within both the arterial and venous confluence conduits, to assist in propelling or slowing the flow of blood through the conduits, thus increasing tor lowering the blood pressure. Pumps positioned thus have ribs along their external surface to prevent migration. A vascular servovalve outlet-regulated shunt or ‘tuned’ shunt as described below in section 2b(2)(h)(h1), entitled The ‘Tuned’ Shunt, and its subsections can be used to automatically tap off a volume of blood to normalize or lower the pressure for all the bloodlines entering and exiting the conduit; however, to increase the blood pressure requires a pump.

While interrupting pulsatile flow, a pump can be left in place as a remote assist device as long as needed, and while demanding reentry, can later be removed endoscopically. Although more costly, if sustaining pulsatile streamline flow is judged warranted, propeller blade, or axial impeller type pumps can be made to rotate their propeller blades in axial alignment with the flow while stationary and not in use.

3. Even though a heart transplant demands an open field, with nine to twelve bloodlines to connect the corresponding vessels of the recipient to the donor, hence, 18 valve connections to perform a heart transplant, the odds for misidentifying bloodlines must be considered. The ability to combine vessels simplifies the surgical field in terms of visually tracking the bloodlines midoperatively, and neutralizes human errors consisting of connecting recipient to the wrong donor vessels, for example. A confluence conduit reduces the number of bloodlines where these enter and depart it.

A confluence conduit such as that shown in FIG. 35A allows the outflow of one or more separately originating inlet bloodlines, each connected to a source vessel, to flow together through a common conduit and flow out the distal end thereof into one or more separately destined outlet bloodlines, each connected to a separate destination vessel. Usually, these connections—readily visualized with the aid of FIGS. 16 and 22—will implement a compound vascular bypass, or sudden switched-organ-type organ transplant by connecting the vessels of a donor organ to the corresponding vessels in the recipient.

4. In direct aortocaval connection, that is, connection of the graft organ, especially a heart with its many vessels, directly to the local aorta and inferior vena cava in the lower abdomen thus eliminating the resistance of gravity compared to the need for venous return to rise up to the chest with the need for an inline pump and accommodating the heart when alternative positions had been eliminated due to disease, injury, or resection, the interpositioning of a confluence conduit to allow perfusion through all of the vessels avoids the disuse atrophy that inevitably will lead to allograft vasculopathy and rejection.

However, because direct pipe-targeting of immunosuppressive medication avoids systemic immunocompromise, the susceptibility to infection and malignancy should prove much reduced if not eliminated. That the number of veins to be connected to the inferior vena cava and arteries to the aorta are reduced to one expedites completion of the procedure and eliminates the arteries as a potential source of procedurally disruptive vasospasm, as addressed above in section 2a, entitled Background. General.

As depicted in FIG. 35A, a confluence conduit must be fed and release blood through separate vessel associated servovalves, each connected to an input or an output vessel where all of the valves respond in exact synchrony by compliance with a common command. To apply a single servovalve at the inlet and another at the outlet of the confluence conduit is not feasible. This is because blood flow through any one valve must remain switchable between continued flow through its normal path to being diverted into continued flow through the corresponding vessel of the other party to the transplant.

A single valve at the inlet into a confluence conduit is limited to the latter path for all of the vessels feeding into it. That is, the ability to switch back to the normal inmate paths has been lost. Furthermore, with this loss there is also lost the ability of the command control microprocessor to partially halt or reverse the continued travel of the diversion chutes in response to the detection of an adverse immune reaction. When each vessel is provided with normal inmate and crossover paths, the detection of such a reaction is easily accommodated by ‘idling’ at the setting reached, or even retraction sufficient to allow the immune transient to abate with or without the release of immune tolerance-inducing medication.

Furthermore, in a heterotopic or intentionally supernumerary transplant, such as shown in FIG. 15A where the valves are to remain at the halfway point in order to supply and drain both organs, the use of a confluence conduit eliminates this capability. At the same time, that each vessel is accessible for the release of medication through the drugline, or accessory channel of its respective servovalve, provides a selectivity for differential treatment which a confluence conduit does not.

Because the confluence conduit makes it possible to segregate a preferred set of vessels for joint therapy, retention of the confluence conduit following transplantation eliminates the time to remove it, and is acceptable if unobtrusive as not to encroach upon neighboring structures. An advantage in implanting a confluence conduit is that the common treatment it affords for the bloodlines connected to it to the exclusion of others is preserved and available to the prosthetic disorder response system for continued therapy with or without changes indefinitely. Where its eventual retention as implanted is contemplated, the confluence conduit is designed to increase in its dimension in step with the patient.

The use of a unified entry confluence conduit confluence conduit as delineated above in section 2b(1)(b)(b5), entitled Heterotopic Double Heart Metered Compound Vascular Bypass, or Switched-organ Transplantation and the Use of a Unified Entry Confluence Conduit to Avoid Disuse Atrophy when the Graft Organ is Positioned in the Lower Abdomen by Direct Aortocaval Connection, and below in section 2b(1)(b)(b16), entitled Direct Aortocaval Connection: Unified Entry Arterial and Exit Venous Confluence Conduits for the Heterotopic Connection of a Graft Heart in the Lower Abdomen, makes possible the implantation of a supernumerary organ, usually a kidney or a heart, so that rather than being connected to its counterpart on a vessel by vessel basis, it is physically independent from its native counterpart. This can make its differential treatment such as medicinal or explantation simpler than were it connected to its native counterpart.

With any heterotopic double heart compound vascular bypass, or switched-organ transplant, when clinical judgment anticipates that the support of an added heart would eventually allow the native heart to recover, and the added heart might be urgently needed for another patient, the original operation should connect the added or graft heart using the valves of the type shown in FIG. 10G, shown implemented in FIG. 15B, to allow the gradual transfer of blood flow from the native to the added heart in such manner that neither heart experiences an interruption in blood flow.

If the added heart proceeds to final end-stage or stage 5 heart failure, the valves on the vessels of the native heart are fully opened, that is, the diversion chutes 18 are fully retracted to confine all blood flow through to the native heart cutting off the graft thus allowing the graft heart to be explanted. If the native heart proceeds to end-stage or stage 5 heart failure, the valves on the vessels of the native heart are fully closed off, that is, the diversion chutes 18 are fully extended, to divert all blood flow through to the graft heart thus freeing the native heart to be explanted, and if judged sufficiently beneficial, repositioning the graft in the mediastinum.

In a pediatric patient, especially a prenate, neonate, or infant, the need for a long-to-short or the reverse confluence conduit switching mechanism such as shown in FIG. 35B almost always relates to a midprocedural span separating the donor and recipient so long that to use an expandable tie-line type conduit as described in section 2a and shown in FIGS. 36A, 36F, and 37D would result in an untenable weight of collapsed expandable fabric to implant. Another expedient to facilitate the compound vascular bypass transplantation of the heart consists of a device for grouping the tiny vessels as described below in this section in the subsection entitled Tiny Vessel Grouped Inlet/Outlet Expandable Confluence Conduits, shown in FIG. 15D.

This is overcome by eliminating the conduit as shown in FIG. 35F, which leaves the valves and therefore the accessory channels in place for follow-up maintenance by the implanted disorder response system. Following placement in the recipient, switching the valves to direct flow-through as shown in FIG. 35F allows the longer confluence conduit to be removed, but at the expense of losing its utility in allowing the confluent blood of the vessels connected to it to be treated alike in contradistinction to any treatment provided to other vessels whether made to flow together thus.

Accordingly, when to span the distance between the donor and the recipient would necessitate the use of a confluence conduit of excessive length, the compound blood switch arrangement shown in FIG. 35B allows the longer to be replaced by a. compact confluence conduit so that the longer of the two confluence conduits can be eliminated and only the short conduit—not necessarily longer than the blood chamber 230 if arterial 230′ if venous, can be implanted. If retaining the shorter of the confluence conduits for implantation is not justified on the basis of its ability to treat only a subset of bloodlines alike, then as shown in FIG. 35F, the smaller of confluence conduits is also removed.

FIGS. 22B and 35F show that the removal of an intervening line or conduit allows the anastomoses joining donor and recipient vessels to be straddled as an alternative passage that allows blood flow to be circuited about the anastomoses. This allows the anastomoses to be relieved of constant flow-through that would retard healing as well as medicated through their accessory channels without the drugs being washed away. Ordinarily, the source vessels will be the same in the organ donor as those of the recipient to which the outlets are connected. FIGS. 35A and 35B show the input bloodlines as connected to each source vessel and the outlet bloodlines as connected to each destination vessel by vascular valves, of which the greater expense can be saved by replacing these with ductus side-entry jackets.

Ordinarily, the individual bloodlines correspond in caliber to the calibers of the vessels each represents and the flow-through cross-section of the confluence chamber is the sum of the cross-sections of the vessels feeding into it. Any necessary adjustments in blood pressure can be applied by adjusting the cross-section of the individual feeder vessel or the corresponding outlet vessel, or the blood pressure for the sum of vessels by adjusting the flow-through cross-sectional area of the confluence chamber of the insertion in the conduit of a flow-through inline pump. The latter can be placed in the longer of switchable long-to-short confluence conduits to propel blood over the distance separating donor and recipient, and since the longer of the two conduits is later removed, the conduit with pump is made available for further use.

While the drawing figures show the cross-sections somewhat larger to support clearer part numbering, the confluence conduit serves as a common connecting viaduct with the same cross-sectional throughflow area as the sum of the contributing vessels. Allowing a simple way to apply adjustments in blood pressure by adjustment in the cross-sectional area is a simple approach offered by confluence conduits, differential therapy primarily with drugs of the confluent as opposed to blood flowing through other lines and a reduction in the critical tracking of multiple vessels in a heart transplant, for example, worthy of emphasis.

Used intracorporeally where a gradual, or metered, change in the cross-sectional area for flow-through is uninvolved as is the case in a compound vascular bypass solid organ transplant, a confluence conduit used to isolate certain vessels for common therapy, for example, is joined to the vessels by means of inline coupling jackets. Inline coupling jackets are used to connect bridging, or tie-lines, addressed below in sections 2b(2)(c) thru 2b(2)(c)(c3d) and shown in FIGS. 36A, 36E, 36F, and 36G, used intracorporeally apart from organ transplantation to replace a segment along a single larger vessel and thus having but a single inlet and outlet not requiring switching ability.

In solid organ switch transplantation, the inlet bloodlines deliver blood from the donor and the outlet bloodlines are connected to the corresponding vessels of the recipient or those corresponding thereto as closely as the anatomy will allow. Significant discrepancies in blood pressure should be diagnosed to remedy the cause rather than to secondarily adjust the pressure. Otherwise, adjustments in blood pressure can be made through the use of side-entry diversion jackets, valves, or servovalves used to tap, or bleed off, a portion of the passing blood, or servochokes can be positioned along the inlet and/or outlet bloodlines of an arterial or venous conduit.

Ordinarily, the flow-through cross-section of the confluence chamber is the sum of the cross-section of the vessels feeding into it, with any necessary adjustments in blood pressure applied by adjusting the cross-section of the individual feeder vessel or the corresponding outlet vessel, or the blood pressure for the sum of vessels by adjusting the flow-through cross-sectional area of the confluence chamber. If necessary, a flow-through pump is inserted in the confluence conduit to change the blood pressure or to move the blood over a longer distance. Such a pump also prevents a disproportionate concentration of more viscid drugs released into the conduit, blending these so that the drugs are equally dispersed into all the blood moving through the conduit.

A confluence conduit can be used to provide adequate surface area to attach a number of accessory channels of which each can be used to directly pipe drugs such as immunosuppressive, anti-inflammatory, chemotherapeutic, adjuvant thereto, or antimicrobial, for example, as treatment separately scheduled for automatic release by the prosthetic disorder response system into the set of vessels connected thereto. These would typically supply a particular end organ, or a portion thereof, or a volume of tissue in contradistinction to other vessels and their targets. This avoidance of systemic dispersal virtually eliminates the side effects that systemic dispersal would have produced, and makes it possible to dispense the targeted drugs in higher concentration than systemic dispersal would allow.

For use in a baby, the shorter line or conduit if to be implanted and not removed, can be made of the expandable fabric described below in section 2b(2)(f), entitled Expandable Structure to Accommodate Growth and Self-adjust in Size in Vascular Segment Prostheses, Lines, and Confluence Conduits Placed in Children, to allow expansion with growth, eliminating a later need for reentry. As indicated, placing a servochoke on a common outlet line allows adjustably increasing the common blood pressure, while placing a servovalve allows tapping off an adjustable portion of the common flow into another vessel.

Sudden compound vascular bypass, or switch transplantation requires that both inlet flow into and outlet flow out of the confluence conduit be switchable, and metered switch transplantation that the volume of flow into and out of each vessel be continuously variable. It may be noted from FIGS. 33A, 33B, 34A, and 35B, that the operator or an assistant can accomplish either sudden of gradual switching action simply through appropriate control of the switching mechanism. Arterial and venous confluence conduits are the same but the reverse in direction.

First, regardless of the number of bloodlines entering and exiting the confluence conduit, connecting vessels through an interposed confluence conduit eliminates the need to separately track and anastomose each of the vessels by direct stitching to its counterpart, materially reducing operative time. If the confluence conduit is eventually implanted, that the vessels leading into and out of it are connected without stitching but rather by means of vascular servovalves means that stitching can be eliminated.

Another inherent advantage is that in connecting arteries of like supply or drainage territory within the parenchymas, or parenchymata, of the graft and the native organ, most often, a kidney, to accomplish a switch, or bypass, transplant, the apportionment of blood flow among the supply or receiving vessels connected thus is self-leveling despite the fact that either, or exceptionally, both, include a variable number of accessory arteries. In this regard. FIGS. 35A, 35B, and 35C thru 35F show the inlet and outlet lines as alike in diameter, which not need be the case. Additionally, the internal diameter of the confluence conduit, larger than any one of the vessels connected to it, makes it possible to position biomarker and/or physiological sensors within it.

As indicated, if for any reason, such as the distance between donor and recipient to be traversed, the exit blood pressure is too low or because a contributing vessel to be discarded with the native heart of the recipient outlets a pressure too high, an inline flow-through pump inside the conduit is run forward to increase, or in reverse to decrease, the rate of flow and sum outlet pressure. One or more thin film strain gauges can be mounted along the interior of the conduit. When the vessels of the graft organ to be connected to the vessels of the native organ to be replaced are proximate, as when the graft organ has been excised, there is no longer a distant donor to which the conduit must span.

The length of the shorter of the switched conduits is then suitable for implantation, preserving accessory channels that will continue to allow the direct targeting of medication to any of the vessels and eliminating the need for vessel-to-vessel anastomosis. Removal of the conduit as shown in FIG. 35F allows bridging around the anastomoses so that the accessory channels can be used to medicate these. To facilitate connection of corresponding vessels of a graft organ maintained by an ex vivo organ delivery perfusion machine to those of the organ recipient, especially where a nonjacketing side-entry connector along the conduit is used to deliver medication or a strain gauge is used to indicate the blood pressure, a longer conduit is used.

FIG. 35B shows a confluent long-to-short or the reverse switchable bloodline which rather than pertinent to a single bloodline, or mainline, as shown in FIGS. 32A thru 32F allows the connection of multiple corresponding arteries or veins between the recipient and donor thus eliminating the need to connect each such corresponding pair directly through a separate origin to destination vessel-addressed bloodline. This expedites solid organ, especially heart transplantation using the switch, or compound vascular bypass, technique not only by reducing the number of separate long to short bloodlines connecting each corresponding pair of vessels between recipient and donor, but provided the same combination of vessels are connected to the inlet and outlet of the confluent line, eliminates the need to separately confirm the correspondence for each pair.

Switchable confluence conduits, addressed in the section to follow, serve much the same purpose as switchable individual bloodlines, which is to allow switching from a long conduit which can span the distance between the donor and the recipient to the short conduit which can be implanted. Alternatively, as shown in FIG. 35F the conduit can be discarded with the anastomoses straddled to allow their treatment while flow is detoured around them. For use in a young patient, a confluence conduit made of the expandable fabric with alternating flats 233 and vaults 234 conformation described below in section 2b(2)(f), entitled Expandable Structure to Accommodate Growth and Self-adjust in Size in Vascular Segment Prostheses, Lines, and Confluence Conduits Placed in Children, shown in FIGS. 15C, 15D, 36A, and 36F, might provide sufficient extendability to eliminate the need for a switchable confluence conduit.

To accommodate growth in an infant without the need for reentry for years, the same fabric is used in a prosthetic replacement for a defective segment along a major vessel such as the thoracic aortic neoarch shown in FIG. 36F. When usable, the expandable conduit is stretched to span the distance between donor and recipient, is implanted in its contracted form, after which it expands with the growth of the patient. Such presupposes that contracted, the conduit will not present a weight that would prohibit implantation. While the goal of expandability is to allow expansion up to adulthood and thus eliminate the need for reentry, the degree of expansion in larger individuals will necessitate reentry during adolescence.

As shown in FIGS. 35A and 35B, confluence conduits used in organ transplantation must be securely connected to the vessels at the inlet and the outlet ends by means of vascular valves, or for metered switch transplantation, vascular servovalves. If to be implanted in a growing child, to avoid the need for reentry over a period of years if not for life, both the confluence conduit and the vascular valves or servovalves connecting it at either end must be expandable. Expandable vascular valves and servovalves are addressed below in Section 6, entitled Description of the Preferred Embodiments of the Invention, in the subsection thereof entitled Expandable Vascular Valves and Servovalves.

In contrast, connection of a prosthetic replacement for a segment along a major vessel to its stumps at either end is by means of inline coupling jackets. As shown in FIGS. 36A, 36B, and 37D, fixed in position by through-and-through stitching of the outer or native stump-grasping half-jackets at either end, the accommodation of growth is through a significant increase in internal diameter of the native stumps allowed by a thick and highly compliant internal lining of a breakdown counteractive-coated layer of viscoelastic polyurethane foam. The tie-line type prosthesis itself expands by the unfolding of its flats 233 and vaults 234 as shown in FIGS. 15C, 15D, 36G and 36F.

To avert the need for reentry, all parts of a tie-line for placement in an infant or small child must be capable of expansion with growth. The inline coupling jackets accommodate growth through a thick foam lining into which the substrate vessel can grow and remain invested without being compressed. Nevertheless, the initial fitting can be snug. To preclude migration, the inline coupling jackets are stitched in place as shown in FIG. 37D. Incorporating sufficient perivascular foam to accommodate growth, the inline jacket at either end of the intervening prosthesis or tie-line these secure in position need not be expansion conformed as is the tie-line itself.

The outer jacket halves clutch the substrate vessel, while the inner jacket halves clutch either end of the tie-line, so that the ends of the tie-line expand into the foam lining at either end must as do the ends of the substrate vessel due to growth. FIGS. 32A thru 32C show venous return flow from the donor on the left to the recipient on the right with a long-to-short or the reverse switchable bloodline. FIG. 35B depicts the extension of the individual switchable bloodline concept to switchable confluence conduits. Individual donor venous bloodlines entering at the left are combined to flow together through the longer of the two confluence conduits while these must span the distance separating the donor and recipient.

This allows what would otherwise have been a number of individual bloodlines as shown in FIGS. 32A thru 32C to be reduced, making the open field through which these must be tracked clearer, and allowing the lines to be medicated and if necessarily pump-assisted collectively rather than individually. Were one such line to require different treatment thus, that line would be omitted from the confluence conduit and provided with an individual long-to-short of the reverse switchable bloodline. Assuming normal vasculature in both donor and recipient, a confluence conduit used to reduce a number of bloodlines will spontaneously apportion the volume of blood flow delivered into each receiving vessel correctly.

That is, even though the outflow of the contributing vessels has been combined, provided the same combination of corresponding vessels in donor and recipient are connected and the vessels are unobstructed, outflow will be self-normodistributed. For this reason, the use of a confluence conduit where the vessels of the recipient are atheromatous or constricted, for example, necessitates that the corresponding unaffected vessels be taken from the donor and included as part of the transplant, or be angioplastied, or be replaced with a prosthetic bloodline with accessory channel to allow the automatic release therein of an anticlotting drip.

This factor of correct vessel-by-vessel volume distribution or spontaneous adaptive flow apportionment from any one of the contributing arteries to the corresponding receiving artery of the donor or recipient applies not only when flow is from a normal number of inlets to a normal number of outlet vessels but when either number is anomalous, such as doubled or abnormally branched. This does, however, presuppose that the accessory, or supernumerary vessels supply or drain the same tissue within the parenchyma as if the vessels had not been anomalous.

At least over the period required to complete the transplant, if one or more anomalously duplicated vessels were stenosed or obstructed while the other remained patent, provided the supply of drainage territory of the sending and receiving vessels are the same, the unaffected of the two vessels would pass the greater volume of blood so that the sum outflow or inflow between the accessory and normal vessel should still approximate that normal. Accordingly, if flow is to or from an organ, typically, a kidney, with accessory, or supernumerary, arteries, even if one or more of the sending or receiving vessels is stenosed or obstructed, the volume of blood to or from the one kidney will equal that of the other despite the difference in the number of vessels among which the flow is divided.

Accordingly, in addition to:
1. Reducing the problem of connection and apportionment of flow between functionally corresponding vessels of the donor and the recipient whether the graft organ or that to be replaced presents normal or anomalous vasculature. Eliminating individual bloodlines, a confluence conduit also eliminates the requirement for individual bloodline distinguishability and addressability, meaning the need to separately run each bloodline from a specific source to a specific destination vessel, so that rather than accommodating branches at the destination through the use of two bloodlines of one branching where each is segregated through the conduit, one bloodline is needed at the entry into the confluence conduit, and unless the supply vessel to the branches at the exit can be jacketed, outlet lines can be provided to each destination branch, and
2. Reducing the number of bloodlines that must be shortened when the graft organ is to be implanted, thereby reducing the procedural time,

Because a confluence conduit has a larger internal diameter than that of its individual bloodlines:
3. Allows the use of a miniaturized single inline flow-through conventional ventricular assist device type pump, eliminating the need for multiple specially produced miniature pumps to accomplish the same function, and exceptionally, one or more of these pumps can temporarily supplement, allow to be postponed, or replace transluminal repair to achieve the volume and pressure needed.
4. Where no common vessel of origin for a number of separate accessory renal vessels, for example, are present, a confluence conduit allows common treatment of the blood moving through the conduit with drugs or other agents through a drugline fused to the outside of the conduit or nonjacketing side-entry connector, for example, to target the organ supplied without the need to separately disperse drugs such as anti-inflammatory, immunosuppressive, oncological, or others to each accessory vessel or systemically as would produce adverse side effects;
5. The larger internal diameter of the conduit also allows placement therein of therapeutic and diagnostic means that would not fit or would cost more to produce than if situated within the contributary bloodlines. The therapeutic means can be used to condition the blood as blended through the conduit, through the release of drugs or other agents or the addition of a radioactive tracer, while the diagnostic means might consist of metabolite or biomarker sensors.
6. Provides the internal space to position diagnostic sensors; and
7. In moving blood from one or more vessels to a different number of vessels, the use of a confluence conduit takes advantage of the spontaneous self-leveling apportionment of blood among outlet vessels differing in caliber to less than a considerable extent without the need to consider the flow-through properties of each.

By the same token, unless impeded by plaque requiring removal, flow originating from a single normal vessel for outflow into vessels anomalous as duplicated, or bifid, for example, or the reverse should apportion the same sum flow between the two. Compound vessel bypass, or sudden switched-organ, solid organ transplantation requiring the connection of corresponding organ supply and drainage vessels between the recipient and donor organs, if an anomalous recipient organ does not present one or more equivalent vessels, or due to a malformity within the recipient organ does not generate the proper outflow, the spontaneous apportionment provided by an interposed confluence conduit can reduce if not eliminate inadequacies of perfusion.

While both obstruction and the reduction of two outflowing vessels where one is supernumerary into one normal receiving vessel represent disruptions to flow, even if not angioplastied, flow into an accessory vessel if stenosed can apportion the same total inflow volume with the open vessel allotted a larger volume of flow according to the degree of restriction imposed by the other. Experience has established that transplantation need not avoid the use of graft organs with supernumerary vessels, and the use a confluence conduit can support the correct volume of outflow from duplicated vessels into the one normal vessel of the recipient. By extension, this concept is applicable to the replacement of an anomalous organ such as a univentricular heart by a heart formed normally, so that flow is properly apportioned between the defective arterial sending recipient heart and the normal returning venous donor heart.
8. In direct aortocaval connection as delineated below in section 2b(1)(b)(b16), entitled Direct Aortocaval Connection: Unified Entry Arterial and Exit Venous Confluence Conduits for the Heterotopic Connection of a Graft Heart in the Lower Abdomen, the graft organ is physically independent from the native organ, making its separate treatment or removal uncomplicated by the need to deal with the native organ to which it is attached and upon which it is dependent. This is not true of a hybrid doubly heterotopic transplant where the heart is positioned in the chest to the right of the native or earlier transplanted, that is, primary heart, but connected to the abdominal aorta and the inferior vena cava with the exception of the pulmonary arteries which are connected directly to the corresponding vessels of the native or earlier transplanted heart across the chest.

Because a nonhybrid doubly heterotopic transplanted heart for the most part functions independently of that primary, the prosthetic disorder system can automatically address either should it malfunction. This can consist of bypassing that fibrillating, for example, and make use of a pacemaker/cardioverter-defibrillator with leads to both hearts which can be used to resynchronize either or both. This in contrast to a heterotopic double heart transplant in which the accessory heart receives and outlets flow through the primary heart so that were the primary heart to fibrillate, the accessory could not independently continue to function normally but would depend upon defibrillation of that primary. However, in a heterotopic double heart transplant, if it is the accessory heart that malfunctions, the automatic system can bypass and attempt to resynchronize it, the primary heart considered to discharge an ejection fraction sufficient to sustain normal circulation until normal function is restored.

The use of confluence conduits is more often intercorporeal with a long to short switchable version that allows spanning the distance between donor and recipient during compound vascular bypass, or sudden switched-organ solid organ transplantation, addressed below in section 2b(1)(b)(b15), entitled Switchable Lines and Confluence Conduits: Long when Intercorporeal, to Short when Intracorporeal, Both Affording Connection of One or More Bloodlines to the Same or a Different Number of Bloodlines with Self-leveling Apportionment of Outflow, and shown schematically in FIG. 35B.

These were addressed under section 2b(1) entitled Intercorporeal Diversion of Blood Flow, and the inline coupling jackets used to connect these to native vessels below in section 2b(2) entitled The Intracorporeal Diversion of Blood Flow. Categorization thus is based upon the majority of potential applications, of which those of inline coupling jacket-connected confluence conduits, if not the majority, are still considerable.

FIG. 35A provides a schematic representation of an individual, or nonswitchable, confluence conduit for an adult, hence, lacking the expansion structure made to accommodate growth in a prenate, neonate, or young pediatric patient. One reason for implanting a confluence conduit is to allow a proper routing of blood to an organ or tissue left hypoxic or anoxic due to the dysplasia, anomaly, agenesis, injury, or disease of its blood supply that mandates its replacement. If necessary, a close by larger artery is tapped to provide addition blood. The confluence conduit also segregates arteries supplying a given organ or tissue for separate treatment.

Most often, this will be the direct pipeline targeting of drugs to be substantially best isolated from nontargeted tissue, such as anticarcinogenic. If the drugs are radioactive, the chain of components along the delivery line leading up to the target tissue, to include the body surface port, drug reservoir, drug reservoir outlet, drug delivery line, accessory channel within if present, and the rest of the confluence conduit, are radiation shielded as shown in FIG. 14B with disintegrating, or temporary, or permanent shielding addressed in U.S. Pat. No. 11,759,186, entitled Ductus Side-entry Jackets and Prosthetic Disorder Response Systems, FIGS. 5 and 6, and Ser. No. 14/998,495, entitled Nonjacketing Side-entry Connectors and Prosthetic Disorder Response Systems, FIGS. 10A and 10B.

As with FIGS. 32A thru 32G, 33A and 33B, shown in FIGS. 35A thru 35F is the venous return of blood from the graft organ to the recipient. The corresponding arterial flow from the recipient to the donor organ, which is the reverse of the venous flow, has been omitted from the drawing both for visual clarity and because the arterial flow had no need for passage through a confluence conduit as would be advantageous were the donor and recipient arteries different in number as is common with kidneys.

Other reasons for the use of a confluence conduit are that confluence allows the flow through each of the arteries to be selectively medicated in common through an accessory channel flowing into the conduit and the practical factor that conjunction thus is neater and easier to deal with in the surgical field. Targeting of specific conduit outlet lines or branches is by providing a drugline to these, and a drugline to the middle of the conduit medicates all outlet blood alike, switching between drugs and the middle or outlet branch always available. Vessels are connected to a confluence conduit by means of vascular valves or servovalves, never inline coupling jackets.

Accordingly, in FIG. 35A, the number of input arteries 194 connected to long confluence conduit 230 at its inlet end by means of vascular valves or servovalves 195 shown to the left and input bloodlines, or tubes 197 is dictated by the normal or abnormal vasculature of the individual patient, hence, is variable, as are these same components in the opposite order at the conduit outlet end to the right, consisting of output bloodlines 197′, outlet inline coupling jackets 195′, and outlet, or receiving, arteries 194.′ Shorter confluence conduit 230′ will be that implanted.

As with all implanted bloodlines and conduits, use of the expandable structure is essential in adults only when the length of the prosthetic segment should exhibit compliance with the pulse. In pediatric patients the antecedent need to use the expansion structure to expand in step with growth inherently satisfies the need for pulse compliance not as a separate consideration. Where one of more native input, or sending, arteries or veins are incompetent or lacking, additional blood supply can be provided expeditiously with little trauma, and if beneficial, without the need for clamping, by using side-entry jackets and a shunt to tap a larger nearby artery or vein.

In FIGS. 35A and 35B, inlet bloodlines 197 could be connected to inlet native vessels 194 and outlet bloodlines 197′ could be connected to outlet native vessels 194′ by side-entry jackets, valves, or servovalves rather than inline coupling jackets. However, unless justified on the basis that the junctions between native and prosthetic lumina must serve the additional function of allowing the adjustment of flow-through to include automatic between the confluence conduit and an alternative path responsive to a different purpose, the use of inline coupling jackets offers a slimmer contour less likely to protrude into neighboring tissue. Referring to section 2b(2)(c)(c1) below entitled Inline Coupling Jackets Proper, and FIGS. 36E, 37A, and 37D, inline coupling jackets as a whole—to include both entry inlet half 199 and outlet exit half 200 and corresponding but reversed exit jacket parts 200′ and 199′—are labeled 195 and 195′.

In addition to the direct pipeline targeting into the conduit of drugs through drug delivery line 8, the confluence conduit can adjust the blood pressure actively by positioning a small pump 167 inside the conduit. In metered compound vascular bypass, or sudden switched-organ transplantation, such a pump can also be used to hasten blending of drugs and/or recipient and donor blood. Absent such a reason to use a pump in a monomorbid, or noncomorbid, adult, the additional expense and procedure to implant a pump and delivery system from body port to confluence conduit can be avoided. A conventional adult conduit inelastic, specifying a narrower conduit will elevate, where one broader will lower, the pressure of the blood passing through it passively, without additional expense.

Whether spontaneously or in response to medication the blood pressure varies more than is normal, an adult-sized confluence conduit with the same elastic expandable outer casing as is used in a pediatric embodiment to allow for growth affords the instantaneity of compliance required, without additional componentry and expense. The attached bloodlines segregated for targeted therapy, a small temperature-changing heating or cooling element can be attached to the outside of the conduit.

Drugs can be directly targeted to any level along the train of components related to a confluence conduit through an accessory channel such as that marked 8 connected to the input arteries 194, or to the entry servovalves 195, or into the connecting bloodlines 197, the conduit itself 196, or the corresponding components on the exit side. Connection of the drug delivery line into a native vessel is through a ductus side-entry jacket, into tissue through a nonjacketing side-entry connector, and into a synthetic component through a leak-free mechanical junction, or a joint plastic or electrically welded.

The structure of bloodline switches differs from that shown in FIG. 32G in providing additional input line sockets and the function is the same as depicted in FIGS. 32A thru 32C Accordingly, in FIG. 35B, input bloodline switch confluence chamber 178 is shown as receiving two inputs and its exit counterpart as feeding into four outlet lines. Renal transplantation often poses the need to anastomose or connect accessory (supernumerary, anomalous, or if polar, aberrant) arteries between either or both donor and recipient organs where the numbers of these do not match.

The longer conduit, used midprocedurally during reciprocal cross-circulation, and shorter conduit which is implanted, both pass through the same volume of blood regardless of the source of any fraction from any one contributory or any one outlet vessel. FIG. 35C shows arterial and venous donor/recipient confluence conduits 230 in concurrent use. With three input and three output vessels, the spontaneous apportionment of the outflow to each of the output vessels is applicable but not as noticeable as it would be if the number of input and output vessels were different in number. The arrangement shown is suitable to simplify the set of separate donor-recipient corresponding vessel connections shown in FIG. 16, especially if druglines 8 and 8′ were used to release medication for application to the three vessels together.

All side-entry devices having at least one accessory channel which can accept a drugline, were it desired to separately medicate each of the three receiving vessels 194 arterial and 194′ venous, then this would be done by releasing the medication through the drugline 8′ of each vascular valve, plunger solenoid driven as shown in FIGS. 7 and 8 in an emergency sudden switch organ transfer, or vascular servovalve driven as shown in FIG. 10A in a metered organ transfer.

FIG. 35C shows a donor/recipient vessel by vessel pressure self-adjusting confluence, or summary, conduit suitable for use to implement the scheme illustrated in FIG. 16B and further expedite completion of the steps depicted in FIGS. 32A thru 32D, consisting of collectively conveying flow from the separate arteries of the diseased or defective native solid organ of the recipient through the confluence conduit to the separate arteries of the replacement or graft organ in the recipient, where the apportionment of outflow among the receiving vessels is ordinarily self-adjusting, hence, without the need for a valve on each, a second confluence conduit used to collectively convey all graft organ return, or venous, flow.

A bloodline confluence conduit is normally used as incorporated into a bloodline-switch FIG. 35, which combines the facility, normodistributed output despite the blending of confluent flow, the ability to place a conventional pump in the long line only while in use, and expeditious removal of the long line. The outlet flow into an organ, typically a good replacement kidney, supernumerary anomalous, or accessory arteries of either or both the recipient and donor kidneys notwithstanding, should display normodistributed apportionment of outlet flow among the arteries. Unless one or more arteries of which the supply is essential to oxygenate a portion of the parenchyma is obstructed—in which case, these should be opened in the catheter laboratory, the apportionment of blood between the kidneys should spontaneously self-level.

Described above in section 2b(1)(b)(b15), entitled Switchable Lines and Confluence Conduits: Long when Intercorporeal, to Short when Intracorporeal, Both Affording Connection of One or More Bloodlines to the Same or a Different Number of Bloodlines with Self-leveling Apportionment of Outflow, a switchable confluence conduit allows quicker shortening of multiple reciprocal cross-circulation bloodlines than does individual bloodlines, or mainlines, once the graft organ has been placed in the recipient.

If necessary, a single inline miniature ventricular assistance-type pump such as those schematically shown as part number 167 in FIGS. 35A and 35B can be used to drive the combined flow over a greater distance than would ejection fraction-unassisted flow. The elimination of having to deal with multiple bloodlines plus the spontaneous self-adaptive flow apportionment among the outlet branches materially reduce procedural time where this is a stress factor for an impaired patient.

In FIG. 35D, to show the corresponding arterial flow from recipient to donor organ through an arterial confluence conduit is considered unnecessary, so that each artery is not shown connected to its counterpart in the donor organ by means of a vascular valve but communication between the corresponding vessels is assumed to be understood without the need to show as much and for this reason, has been omitted for clarity. In this, the sum volume of flow through the conduit is distributed among the recipient vessels according to the flow rate through each outlet branch.

FIG. 35D shows a simpler scheme than that depicted in FIG. 16 achieved with the aid of confluence conduits by limiting donor-recipient vascular connections to those supplying the upper body, those to the lower body clamped just before transection and anastomosis. To group the independent interconnections between each corresponding vessel of the donor and recipient through confluent flow through a common channel, or confluence conduit, shown in FIG. 16, visually simplifies the operation where the outflow is self-leveling among the outlet bloodlines and reduces the procedural duration to a degree that can make the operation tolerable to a frail patient. Moreover, as shown in FIG. 35A, arteries and veins separately collected together thus can be differentially medicated thru drugline 8 in relation to arteries and veins not included, and adjusted in pressure and rate of flow by a conduit inline pump 167.

The vascular valves allow a heart transplant, for example, to be performed with no interruption in the flow of blood and without general anesthesia. Should the distance needed midprocedurally between donor and recipient require a confluence conduit much longer than would be implanted, the long conduit can be exchanged for a short implantable confluence conduit when the graft organ is to be placed in the recipient, and the exchange can also be accomplished with no interruption in the flow of blood. Switching between confluence conduits is the same as switching from an individual long to an individual short bloodline-through the interpositioning of inlet and outlet bloodline switches. Switching of an individual bloodline is shown in FIGS. 32A thru 32C, and bloodline switches are shown in FIGS. 33A, 33B, and 35C

A compound vascular bypass, or switched-organ heart transplant is performed by connecting corresponding vessels at the highest level and using these connections to supply the branches. That is, where there appears a choice between connection at the highest and a lower level, the highest is used as simplest. Upon initiating reciprocal cross-circulation, if passing between donor to recipient through a line so long that an interval of donor-recipient nonperfusion would occur, the line is primed with third party blood. The great vessels are generally large enough in diameter that if to traverse the distance across a long line demands greater pressure, the line should large enough to admit an inline assist pump.

The same situation with smaller caliber vessels may require that flow through the arteries be passed through one confluence conduit flow through the veins through another confluence conduit, each large enough to accommodate an inline pump, part 167 in the drawing figures. A confluence conduit also simplifies the procedure in that medication whether immunosuppressive, anti-inflammatory, and/or antimicrobial, for example, can be delivered to the vessels of which the flow is collected through a common drugline, part 8 in the drawing figures.

FIG. 35E shows the penultimate second step in solid organ transplantation for using the bloodlines of a confluence conduit as bypasses around each anastomosis as shown in FIGS. 22B and 35F.

Therefore, the receiving organ is supplied with the correct volume of blood regardless of the apportionment thereof among different vessels. That is, the confluence conduit cross-levels the total volume of blood as between the number of its tributaries and outlets. Hypoxic areas within the parenchyma of the recipient organ are disregarded as pending excision; unless a badly needed donor organ presents such a defect which cannot be durably corrected readily, it will usually be disqualified for transplantation. An organ with a lesser defect such as accessory renal arteries is not normally discounted; however, the surgery to implant is considerably reduced in complexity allowing repair more readily and perhaps more durably through the use of a confluence conduit.

### Use of Confluence Conduits in the Replacement of a Heart Severely Malformed at Birth

FIGS. 15 and 16 are simplified schematic representations of the vascular connections in a switch, or bypass, orthotopic heart transplant midway through the procedure, or in a heterotopic double heart transplant at the end-point, both figures assuming a normal internal structure in the native heart of the recipient. While this will almost always be true of the native heart in an adult with end-stage heart failure, making FIGS. 15 and 16 directly applicable, in a pediatric patient, a heart failing due to congenital defects more serious than a stenotic valve, patent ductus arteriosus, or septal defect, for example, but rather extensive internal malformity such as a univentricular heart would require replacement.

Compound vascular bypass heart transplantation in a tiny patient is with the aid of a grouped inlet/outlet confluence conduit which eliminates the need to separately connect each inlet and outlet vessel with a tiny vascular valve that to produce each involves considerable expense and to use considerable microsurgical adeptness. This device flows all the vessels inlet past a common diversion chute ordinarily driven by a linear plunger solenoid, the use of more costly servovalves as in metered vascular bypass transplantation avoided due to the immaturity of the immune system.

The confluence conduit and associated extension arms and bloodlines are made using the expandable structure described below in section 2b(2)(f), entitled Expandable Structure to Accommodate Growth and Self-adjust in Size in Vascular Segment Prostheses, Lines, and Confluence Conduits Placed in Children, and not using vascular servovalves which eliminate the need for such function, can be controlled in flow rate as a tune shunt described in below section 2b(2)(h)(h1), entitled The ‘Tuned’ Shunt, and its subordinate sections.

The appropriate standard for repair is that the end product of therapy should be normal circulation, something not provided by conventional reconstructions, which are also significantly traumatizing. To replace such a heart using the metered compound vascular technique, the operation is begun as soon and the normal heart implanted in as little time as possible. Confluence conduits such as shown in FIG. 35A are used to connect the vessels of either heart supplying blood separately from those connecting the vessels providing venous drainage. The self-leveling advantage afforded by the confluence conduits dispels any ambiguity in making these connections.

As soon as perfusion has been passed entirely through the graft organ, even before the native heart of the recipient and the donor heart have been removed, the circulation of the patient will have been instituted as normal. The operation is much safer than is conventional reconstruction or replacement, and the implanted disorder response system is left to monitor the patient and apply remedial measures as appropriate and automatically release immunosuppressive medication, much alleviating the apprehension associated with organ transplantation. Due to the legality of abortion, the pool of replacement hearts can be much increased, eliminating the need for a compound vascular heterotopic double heart transplant to allow the use of a less than perfect graft heart in a neonate.

Exceptionally, and only if judged essential, a compound vascular bypass double heart heterotopic bypass transplant is accomplished in three steps, the first to orthotopically replace the malformed heart by bypass transplant as indicated in this figure; the second to afford the recipient an interval to stabilize, if necessary, with the aid of a mechanical circulatory assist device; and the third to transplant a second competent heart into the recipient to work together with the first transplant as depicted in FIGS. 15, 16A, and 17. The competency of the first graft heart confirmed, the placement of a second heart or a mechanical assist device is for the time being, called off.

While following a organ transplant using the compound vascular bypass technique the vessels can be anastomosed and bloodlines 9 removed, the jackets with druglines 8 attached and continuous with the valve accessory channels remain to deliver drugs detected as necessary by implanted sensors so that the master controller is signaled of the need for direct pipeline targeting of a medicinal or electrostimulatory therapy as necessary. FIG. 15A also represents the half-way point in an orthotopic metered switch heart transplant before the valves are adjusted to direct all blood flow through the donor heart. To perform a switch orthotopic heart transplant, the native heart is then removed and the graft organ set in its place.

Leaving the vascular diversion servovalves at the half-extended position or somewhat aside thereof to accommodate a graft organ of different size, allows the native heart to remain and the donor heart to be positioned beside it, for example, some volume of the right lung removed to accomplish this. Whether such a metered switch heterotopic for double heart transplant places the added heart in the abdominal cavity or nestled in the iliac fossa, for example, it is noteworthy that the flow of blood through all the significant vessels of both the native and donor hearts is that assigned by nature.

Downward directed arrowheads denote venous return, upward directed arrowheads ejection, and the arrowheads along the bloodlines indicate the direction of flow. The same connections apply regardless of where the donor heart is positioned in the recipient; this configuration provides a distribution of the blood-moving load proper for the chambers and the pattern of flow normal for the vessels. Preservation thus is best with the donor heart positioned as shown, but where unavoidably, placement must be lower in the body, function will still be much closer to normal than were connections made to local vessels. Direct connection to the inferior vena cava and abdominal aorta, for example, results in much flow past the offline transplant, and therewith, disuse atrophy.

The added heart functioning as an adjunct or reinforcer of the native heart, only the native heart is directly connected to the rest of the circulatory system. With the servovalves connecting the bloodlines set to favor both equally at the halfway point, each heart contributes half to the overall volume of blood ejected. In fact, the valve setpoints are shifted to favor the hearts according to the competency, that is, the ejection fraction, or absolute stroke volume, of each. Thus, when the hearts are equal in this regard, the servovalves connecting the mainlines are set at about the halfway point and can be intermittently shifted between an almost entirely open and closed position to allow either heart to receive the full rather than half the normal volume of blood.

Venous return is from the native to the added heart, while ejection is from the added heart to and through the native heart, so that with the hearts positioned as shown, the overall flow of blood is clockwise. The metered switch pattern of flow shown was initiated after the valve-jackets and communicating lines were connected to the donor heart on the left while still in the donor on life support. This reciprocal cross-circulation was continued long enough to accomplish a measure of immune tolerance induction as integral in the process of gradually transferring the heart from the donor to the recipient.

Because unlike paired organs such as the kidneys or lungs the heart cannot be taken from a living patient, this process can continue over a longer duration. The donor heart is then harvested and placed in the recipient without disconnecting the lines or valves, which are to be preserved for the follow-up direct pipe-targeting of drugs by the implanted automatic disorder response system which can continue for life. Connected thus and alternated as indicated, the ejection and venous return of two hearts add with a time lag smallest when adjacent in the chest as shown and increased as the donor heart is positioned farther down in the body.

In FIGS. 16 and 17A, the native or earlier orthotopically transplanted heart on the right is undisturbed in its connections to the rest of the circulatory system, the diversion servovalves extracardiac, placed on its in- and outflow vessels. The bypass, or metered switch, transplanted heart on the left in the figure communicates with the circulatory system by connection to and through the vessels of the native heart shown on the right. That flow through the graft is ‘natural’ is intended to eliminate disuse atrophy as occurs in the direct connection of a redundant heart to the inferior vena cava and abdominal aorta, for example, as has often been done in immunological research, for example.

The connection of the added to the native heart is accomplished by truncating a metered switch orthotopic heart replacement at about the halfway point, then clamping, harvesting, and implanting the donor heart without disconnecting the blood diverting lines. A double heart transplant is always metered, never sudden switched. In a double heart transplant, the vascular servovalves-those of the recipient at the right end of the connecting bloodlines and those of the donor at the left end-placed while the donor on life support remains intact-mediate the compound vascular bypass of the native organ and thereafter are not removed but rather remain to channel the blood as shown and allow the direct pipeline targeting of drugs through any valve.

For visual clarity, connecting bloodlines in the chest view of FIG. 17 were limited to three—the aortae, venae cavae, and right inferior pulmonary veins. As shown in FIG. 16, there are, however, actually nine major incurrent and excurrent major vessels, hence, nine mainlines, or bloodlines, eighteen servovalves, and 18 druglines, each routed to at least one accessory channel in at least one of the valves. In contrast to the lung transplant depicted in FIGS. 22A and 22B, the stumps are distant and if wished retained can be connected with side-entry-secured communicating catheters. Thus, whereas in a switch double heart transplant the 18 valves are best left in place rather than replaced with simpler side-entry jackets before closing, in an orthotopic switch heart transplant, donor and recipient stumps can be anastomosed allowing 9 of the valves to be removed.

### Tiny Vessel Grouped Inlet/Outlet Expandable Confluence Conduits

If a compound vascular bypass heart transplant in a prenate, neonate, or toddler had to involve connecting each of the corresponding vessels of the donor and recipient, the duration of the operation would be much increased, the cost of the tiny vascular valves needed would be considerable, and the expertise in their use much more demanding than if multiple vessels could be combined to pass through a common larger diversion chute.

The advantages of this approach include sparing the already impaired patient a longer ordeal and the need for valves or servovalves tiny in size, which would be difficult and costly to produce. For the reasons stated above, a solid organ transplant in a small pediatric patient will ordinarily be sudden switched; however, where circumstances recommend, the metered switch method can be used. For this reason, subject to clinical judgment, the diversion chutes shown in FIGS. 15C and 15D could be either solenoid or servovalve driven.

The tiny vessel grouping procedure is an interim measure to expedite a compound vascular bypass heart transplant in a perinate, that is, a late prenate, neonate, or early infant bom with a heart so defective as to be surgically irreparable as to instate normal circulation if survivable at all, and in so doing, interdict the global degradation in development addressed above in section 2a(1), entitled Congenital Defects of the Heart and Other Organs—The Importance of Prompt Intervention.

The grouping devices have the internal elasticity to accommodate growth over a significant interval during which the patient can become sufficiently robust to allow a minimally invasive placement of bypasses from the entering grouping device arteries to the exiting arteries. Because the arteries are grouped, this can be accomplished through small incisions to include the use of a robot and without the risks of cardiopulmonary bypass, cardioplegia, and general anesthesia.

Unlike the research testing method described below in section 2b(2)(i), entitled Vascular Servochokes, in the subsection thereof entitled Means for Determining the Optimal Blood Pressure for the Endothelial Uptake of Disorder-pertinent Drugs and Sufficiency of Responsive Secretory Function, here the procedure must be performed on the patient, not on a deceased surrogate sustained on extracorporeal membrane oxygenation past death, and for that reason, the full armamentarium for dispelling vasospasms and arrhythmias to include inotropes and synchronization circuitry with personnel experienced in their use must be at hand.

In these patients, the immature condition of the immune system allows the use of sudden rather than metered switch transplantation so that the valves are driven by highly damped nonsparking linear plunger solenoids. The number of confluence conduits used in a heart transplant likely three or four, precise synchronization requires that all of these solenoids be actuated in precise synchrony from a common switch. For use in a prenate, neonate, or toddler, the impulse to reduce the duration of the operation and the relative immaturity and weakness of the immune system recommend the sudden switch method whereby the diversion chute drivers are bistable linear plunger solenoids.

Especially intended for performing late prenatal, neonatal, infant, and toddler compound vascular bypass heart transplantation, grouped vessel inlet/outlet confluence conduits allow multiple like vessels, meaning those carrying oxygenated or deoxygenated blood, to be bunched together before their outflow is separated into diverted and nondiverted fractions or passed entirely straight ahead or into the confluence conduit. Shown in FIGS. 15C and 15D, grouped vessel inlet/outlet confluence conduits are meant for use to manually connect the inlet and outlet extension arms of the conduit to their respective source vessels without the need to interrupt the flow of blood in prenate, neonate, and early toddler heart transplantation where as shown in FIG. 16, the number of vessels is nine without separately connecting the supraaortic vessels.

The object in grouped vessel inlet/outlet confluence conduits is to allow the grouped vessels to be connected and passed into and out of the confluence conduit by simply closing a jacket around them. Whenever handling arteries, it is important to be prepared to counteract vasospasm and arrhythmias, and in a heart transplant this is especially so. Automatic resynchronization circuitry should be turned on throughout the operation, arrhythmia-counteracting agents directly pipe-targeted to the artery or arteries being handled as necessary, and if applicable, to other arteries involved in the procedure as may be affected. Antispasmodic, or spasmolytic, agents include nitroglycerin, nifedipine, diltiazem, and verapamil.

An additional small background systemic dose can also be recommended. This because a significant systemic dose will result in an increase in the diameter of the veins with a consequent reduction in the rate of venous return as well as the diameter and exertion of the ventricles. Possible complications include bradycardia and hypotension, both reversible. Grouped vessel inlet/outlet confluence conduits are introduced here and described in greater detail below in section 6, entitled Description of the Preferred Embodiments of the Invention.

Switchable grouped vessel inlet/outlet confluence conduits for use in prenates, neonates, and toddlers should adhere to the guidance discouraging the positioning of an inline axial pump 167 in the shorter of the two conduits provided above in section 2a, entitled Background of the Invention. General. Specifically, the longer of the two conduits is used only during organ transfer and then discarded whereas that shorter is implanted. Nonpulsatile flow results in developmental shortcomings so that an inline pump 167 would interfere with the passage of pulsatile flow past it. FIG. 15C tracks FIG. 15A with respect to the upper of the four valves shown, and FIG. 15D shows these connections in more detail than does FIG. 15C.

The need to connect numerous tiny vessels arises in a prospective fetal, neonatal, or younger toddler heart transplant in whom the nine or more vessels of the recipient are to be connected to the corresponding vessels of the deceased donor kept on life support past death are tiny, so that to directly connect each by side entry would take much time as well as require considerable microsurgical skill and extend the stress imposed on the already impaired patient. The venae cavae large and just two in number (and the azygos veins omitted for simplicity), the need to group vessels pertains only to those vessels carrying oxygenated blood less the pulmonary arteries, which despite designated as arterial, actually carry deoxygenated blood.

These convey the inflow from the cardiac (such as the pulmonary veins) and supraaortic vessels of the recipient into the systemic and pulmonary circulation of the recipient, which consolidated, pass behind the recipient flow diversion chute in the upper right-hand corner. At the same time, having passed through the donor valve at the upper left-hand corner through the upper expanding confluence conduit, the corresponding output from the donor heart is diverted by the recipient diversion chute in the upper right-hand corner, both recipient and donor components then flowing into the systemic and pulmonary circulation of the recipient.

In this way, the tiny vessels of both the late-term prenate, neonate, or infant which tiny would otherwise present a hindrance to valve separately are grouped for flow through common valves. Referring to FIGS. 15C thru 15E, to group multiple vessels in order to pass or flow through a common diversion chute into a confluence conduit without interruption in the flow of blood can be accomplished even though the group must be transected upstream and downstream to the diversion chute. The object being to avoid the need to connect each of numerous vessels separately, the long-to-short line switching scheme shown in FIGS. 32D thru 32C, 33A, 33B, and 35B must be eliminated from consideration as shifting the need for individual connection up- and downstream such as shown in FIG. 35A.

In a compound vascular bypass heart transplant, where three or four confluence conduits and a venous line are needed, the risk of dysrhythmias and vasospasms is significantly reduced due to the fact that once the grouping devices have been placed manually, during which time the risk is greater, the organ transfer from the circulatory system of the donor into that of the recipient is administered by the automatic disorder response system, so that all of the valves are switched simultaneously. This factor notwithstanding, resynchronization means and inotropes are kept at hand. A more detailed description of the mechanisms to which reference has been made will be found in section 6, entitled Description of the Preferred Embodiments of the Invention, in the subsection thereto entitled Tiny Vessel Inlet/Outlet Grouping Device.

2b(1)(b)(b15). Switchable Lines and Confluence Conduits: Long when Intercorporeal, to Short when Intracorporeal, Both Affording Connection of One or More Bloodlines to the Same or a Different Number of Bloodlines with Self-Leveling Apportionment of Outflow

Short-to-long switchable confluence conduits made with the expandable structure described below in section 2b(2)(f), entitled Expandable Structure to Accommodate Growth and Self-adjust in Size in Vascular Segment Prostheses, Lines, and Confluence Conduits Placed in Children, and shown in FIGS. 15C, 15D, 36A, and 36F, have a place in accommodating growth in a young patient. In some cases, the use of expandable fabric can eliminate the need for long-to-short or the reverse switchable confluence conduits.

In a long-to short (or the reverse) switchable confluence conduit such as shown in schematic form in FIG. 35B and addressed in this section, additional pressure to propel the blood from the recipient to the donor through the long line if needed is obtained through an inline flow-through pump, part number 167 in the longer of the two conduits. When the switchable conduit is switched to the shorter conduit, the longer conduit with pump remaining is removed, flushed through to remove any accreted clot or pathogens, and made available for further use. The longer of the switchable confluence conduits can be as long as necessary, incorporate a pump such as a small pump used in a ventricular assist device, and the short to be implanted incorporating the expandable structure, to allow expansion for growth.

FIG. 35B shows a long-to-short or the reverse switchable confluence conduit which allows rapid shortening of the midprocedural reciprocal cross-circulation confluence conduit through its replacement with a shorter confluence conduit suitable for implantation once the graft organ has been placed in the recipient. A switchable confluence conduit allows the input of one or more vessels of an organ donor or recipient to blend and then outlet to the same or a different number of corresponding vessels in the organ recipient or donor.

FIG. 35B shows these features combined in a switchable confluence conduit. FIG. 35B, intended to illustrate the utility of a confluence conduit in supplying a number of outlet vessels different in number than those inlet as in a kidney transplant involving a native and/or graft organ with anomalous, or accessory, vessels, shows only two input vessels, but several can be input or output. The switch to a short confluence conduit is no different than the sequence of steps shown for an individual bloodline in FIGS. 32A thru 32C.

A switchable confluence conduit thus differs from an individual bloodline switched device as described above in section 2b(1)(b)(b15), entitled Switchable Lines and Confluence Conduits: Long when Intercorporeal, to Short when Intracorporeal, Both Affording Connection of One or More Bloodlines to the Same or a Different Number of Bloodlines . . . , only in the receipt into the bloodline switch confluence chamber 178 of more than one input vessel where the output can be one or a number of outlet lines.

Short switchable confluence conduits as shown in FIG. 35B relate to switchable short bloodlines as shown in FIG. 32A and described above in section 2b(1)(b)(b13), entitled Bloodline and Drugline Switch Devices: Long when Intercorporeal to Short when Intracorporeal to Accommodate Growth, as switchable long confluence conduits are to long switchable individual bloodlines as shown in FIG. 35B. The mechanism shown can be used to switch individual lines or confluence conduits of different lengths. The use of a switchable confluence conduit in terms of removing a longer upper conduit is depicted step by step in FIGS. 32A thru 32F. Except for FIG. 35C, the drawing figures have all been reduced in visual complexity by depicting only the venous lines or confluence conduits. The arterial lines and confluence conduits are the same but flow in the opposite direction.

That is, the arterial bloodlines are wrapped around the arterial conduit as are the venous bloodlines but connected to the arteries of the graft organ. Both cross over the inter-intra-corporeal distinction in that the longer of the two switchable lines or confluence conduits is used during reciprocal cross-circulation as described above in section 2a, while the shorter-part number 171 which along with the input 197 and output 197′ bloodlines for use in an infant are made of the expandable fabric described below in section 2b(2)(f), entitled Expandable Structure to Accommodate Growth and Self-adjust in Size in Vascular Segment Prostheses, Lines, and Confluence Conduits Placed in Children, shown in FIGS. 15C, 15D, 36A, and 36F—can be implanted rather than removed with anastomotic segment bypasses created as shown in FIGS. 35C thru 35F when its vessels are anastomosed in the recipient.

In FIG. 35E, the graft organ has been transferred into the circulatory system of the recipient, anastomosed into the recipient, dependency upon the donor having been eliminated. Circulation is now through the anastomotically reconstituted supply and drainage which bypass the confluence conduit. At this point, the confluence conduit can be discarded, implanted, or its outlet bloodlines used to provide bypasses around each of the anastomoses. The implanted endpoint is shown in FIG. 35E and the conduit removal endpoint is shown in FIG. 35F.

The use of a confluence conduit allows combining multiple bloodlines to flow together through the conduit to avoid what would otherwise be a number of separate bloodlines that would have to be individually shortened—or to accommodate growth after having been implanted, individually lengthened as shown in FIGS. 32A thru 32C. When to be implanted in an infant, the expandable smaller lower venous bloodline 196′ along with its input 197 and output 197′ bloodlines is around 5 to 7 centimeters extendable to three if not more times its unexpanded length.

If implanted, the bloodline switches which cannot be removed without interrupting the flow of blood must be wrapped around in an implantable padding material. Preservation of the conduit is to allow the joint treatment of the vessels it includes; otherwise, its implantation is best avoided as risking discomfort due to encroachment upon neighboring tissue, especially since the bloodline switches cannot be removed without interrupting the flow of blood, which depending upon the organ transplanted, could provoke a desynchronization or a stroke.

The sequence of steps in switching between a long conduit to a shorter one is equivalent to the steps in doing so with switchable bloodlines as depicted in FIGS. 32A thru 32F as described in the preceding two sections. As shown in FIGS. 35D thru 35F for a unitary, or individual, confluence conduit equivalent to the shorter of the conduits in a double or switched-organ capable version, the end product of its use can be an anastomotic bypass, which is to say, switchable between flow through the anastomotic segment or around it with drug delivery in either state. If the length of the longer of the confluence conduits exceeds that implantable, switching to a shorter confluence conduit makes this, hence, anastomosis compatible bypass, possible.

FIG. 35C shows switchable longer or stretchable conduits, that arterial from recipient to donor above and that venous from donor to recipient below. FIG. 35E shows the step in bypassing the anastomoses between the steps depicted in FIGS. 35D and 35F. In FIG. 35F, if implanted rather than removed as shown, the confluence conduit allows the continued release in common of medication into the outlet vessels included. If removed and the bypasses created as shown, the master controller can release medication differentially into each of the anastomotic segments, their bypasses, or both. Medication in this example would ordinarily include statins, anti-inflammatoires, and antimicrobials.

In a neonate or infant, the bypass tubing is also made of the expandable fabric described below in section 2b(2)(f), entitled Expandable Structure to Accommodate Growth and Self-adjust in Size in Vascular Segment Prostheses, Lines, and Confluence Conduits Placed in Children, shown in FIGS. 36A and 36F. With the servovalve diversion chute fully extended as shown in FIG. 5 so that all upstream, or oncoming blood flow is diverted out through the valve side-stem 9, drugline, or accessory channel, 8 empties through opening 31 beneath the distal tip of the diversion chute into the diverted anastomotic segment, while drugline 8′, as shown can release medication to wet the substrate ductus adventitia, or were it extended, empty into the anastomosis bypass.

Drugline 8′ can be duplicated to provide drug release against the adventitia, into the flow out through the bypass, or both. When diversion chute 18 is fully retracted so that no blood is diverted out through the side-stem 9 and into the bloodline, or mainline, drugline 8 releases medication or a maintenance solution into the side-stem 9. At any level of extension into the native lumen, diversion chute 18 redirects an equivalent volume of blood out through the side-stem 9, with the release of the medicinal or maintenance solution into the native lumen at a constant volume.

In switch, or compound vessel bypass solid organ transplantation, the recipient and donor may be separated by a distance that requires connections between corresponding vessels which require bloodlines too long to implant. When an expandable confluence conduit cannot be stretched to the length required, a solution is to provide a switching scheme that allows flow to be transferred without interruption from a long conduit to one of implantable length.

A switchable confluence conduit provides a longer confluence conduit to span the distance between donor and recipient which is removed after the flow of blood therethrough has been seamlessly switched by means of a blood switch such as that shown in FIGS. 33A, 33B, and 35B thru 35F to another confluence conduit short enough to be implanted. FIG. 36F shows a flats 233 and vaults 234 expandable fabric anatomical tie-line type prosthesis joined to its arterial vessels by inline coupling jackets-one at the inlet, three at the supraaortic outlets, and one at the outlet. Inside the connecting outer half inline coupling jackets at each end, compressible foam between the outer shell and the outer surface of the substrate native vessel, usually the adventitia of the thoracic or abdominal aorta, compresses to accommodate growth.

When the distance between donor and recipient falls within the extendable length of the expansion configured conduit, a separate longer upper conduit is unnecessary. The envelope, or shell, of both inline coupling jackets and the prostheses or simple cylindrical lines, or tie-lines the jackets secure at either end differ in that the outer half-jackets accommodate growth with their thick internal layer of foam, whereas the inner half-jackets do so in relation to the expandable material. Expandable vascular prostheses, bloodlines, tie-line type prostheses, druglines, and confluence conduits make it possible to avoid the need for reentry to accommodate growth for years if not for life.

When placed in an adult, once set to what would appear the required length, these have the advantage or spontaneously adjust to the proper length. In this case, the operator manually pulls open the prosthesis to somewhat undermatch the dimensions of the innate vessel about to be replaced, and the prosthesis when placed spontaneously expands to dimensions necessary. FIGS. 32A thru 32C show the venous return flow apart from the concurrent arterial flow through long and short lines connected by line switches in use during a compound vascular organ transplant. The longer of the two lines extends from the donor on the left to the recipient on the right.

Line switches permit exchanging a longer line without the expansion configuration of a prosthesis for placement in an infant needed to span between donor and recipient during the operation where the distance exceeds that to which the smaller expandable prosthesis or line cannot extend. The shorter expandable line will be implanted. Flow is switched to the shorter line, whereupon the longer is removed. The removal of the longer of the two confluence conduits between FIGS. 35B and 35D is equivalent to the removal of the longer of the two simple lines in FIG. 32B. The benefit in bloodline and conduit switches is that these allow switching between the long and short versions without interruption in the flow of blood, hence, eliminating an interruption in perfusion of the graft organ.

To the advantages of individual nonswitchable confluence conduits, one switchable adds the ability to position within the longer a small ventricular assist inline flow-through type pump to assist in propelling the blood over the longer distance separating donor and recipient or otherwise adjust its pressure without the need to alter the caliber of the conduit. To increase the pressure thus will require a source of supplementary blood. When time allows, blood donations from the recipient in advance of the procedure reduces the matching variables.

In some cases, an expandable fabric-based confluence conduit can eliminate the need for a switchable long to short conduit. However, where the conduit in unextended form is to be implanted in the recipient, this may result in a mass unsuitable for implantation. Especially in a neonate or infant, when a single conduit such as that shown in FIG. 35A cannot be stretched to span the distance between the donor and recipient and then implanted, if necessary, a long nonexpandable conduit with inline flow-through pump, part number 167 in the upper conduit shown in FIG. 35B can be as long as necessary. The cross-section of a confluence conduit should be the sum of the cross-section of the vessels it supports.

Then when the transplant organ has been harvested for placement in the recipient, the longer conduit is removed much as is shown for an ordinary individual catheteric line in FIG. 32B. The bloodline switch, part number 169 is then used to transfer the flow of blood through the lower conduit, in this case, made of the expandable fabric described below in section 2b(2)(f), entitled Expandable Structure to Accommodate Growth and Self-adjust in Size in Vascular Segment Prostheses, Lines, and Confluence Conduits Placed in Children, and shown in FIGS. 15C, 15D, 36A, and 36F. Shorter and smaller, the expandable conduit will often be implantable and allow growth without the need for revision.

FIGS. 32A thru 32F show how a long-to-short venous switchable bloodline as shown in FIGS. 32G, 33A, and 33B, and addressed in the preceding section can be minimized after having been switched to the short bloodline which will be removed for implantation. In FIG. 32C, the sending venous valve at the lower right is reattached as it is rotated-indicated by the circular arrow top center-into a position above the receiving venous valve-indicated by the sweeping left to right with the inlet and outlet bloodline switch valves remaining attached arrow from its original to its ending position at a distance above the venous receiving valve to allow the venous stumps to be anastomosed.

In FIG. 32D, the valves remain extended with the valve diversion chutes extended so that the venous blood flows through the upper valve, through the bypass. In FIG. 32E, the diversion valves have been retracted so that blood flows through the anastomosed segment, which allows the sending and receiving bloodline switches to be removed. In FIG. 32F, a bypass without bloodline switches is attached and the diversion valves extended so that blood flow around the anastomosis through the bypass. FIGS. 22B and 32E show how both the synthetic bypass and native passageways can exist simultaneously so that switching the valves allows either to serve as a backup for the other should either become impaired.

The need for such action is signaled to the implanted prosthetic disorder response system master control microprocessor by pertinent physiological indicia sensors implanted in the organ drained by the venous line and is automatically responded to by the release of drugs through the druglines, or accessory channels, is executed by master controller in accordance with its prescription-program. Switching thus may also be triggered on the basis of impaired flow through prosthetic or the native passageway.

If to be implanted, the shorter of the two confluence conduits incorporates features that will allow it to remain dependable in the internal environment indefinitely. As indicated, when implanted in a neonate, infant, or small child, the conduit must be expandable to allow growth without the need for reentry over a period of years if not ever. As described below in section 2b(2)(f), entitled Expandable Structure to Accommodate Growth and Self-adjust in Size in Vascular Segment Prostheses, Lines, and Confluence Conduits Placed in Children, shown in FIGS. 36F, expandability in diameter and length is imparted through the material and structure delineated in section 2b(2)(f). The structure described there is also of value in supporting pulsatile flow.

Further in a neonate or infant, as shown in FIGS. 32F and 35F, a shorter conduit to not just allow implantation but serve as an anastomotic bypass that will allow the master control microprocessor to switch the release of medication between the anastomotic segment, the bypass, or both and allow growth over a period of years is also made of the expandable fabric. FIG. 35B shows a switchable long-to-short or short-to-long confluence conduit, where only the one or the other is in use at a given time to provide the same utility as the individual confluence conduit shown in FIG. 35A.

When it is suspected that the shorter conduit with bloodlines will not be able to extend to the adult size, the shorter conduit should not be removed so that a short-to-long conduit can be inserted into the upper position, preferably endoscopically without the need for incision to create an open surgical field. In this circumstance, the upper sockets of the inlet and outlet bloodline switches should be packed with a material that will prevent the intrusion into the sockets of extraneous matter that would impede this process.

FIG. 35B shows a switchable long-to-short or short-to-long confluence conduit suitable for connecting a different number of inflowing and outflowing conduits without the need for anastomoses. If a line made of the expandable fabric described below in section 2b(2)(f), entitled Expandable Structure to Accommodate Growth and Self-adjust in Size in Vascular Segment Prostheses, Lines, and Confluence Conduits Placed in Children, shown in FIGS. 36F, which can be extended over the distance separating the donor from the recipient, then a switchable confluence conduit is not needed. Where this is not the case, the upper longer line is a conventional length of tubing equal in cross-section area to the sum of the input vessels, while the shorter lower line is a conventional length of tubing or if to be implanted, is made of the expandable material jut specified.

While not preferred over direct donor-recipient side-by-side compound vascular bypass solid organ transplantation, when the excised graft organ must be transported, FIGS. 35A and 35B show embodiments that facilitate the connection of a different number of corresponding vessels between the donor and recipient no different than were both parties available during the use of an organ transport ex vivo perfusion machine such as Trans Medics Incorporated, Andover, Massachusetts, Organ Care System® or the portable ventilator and cardiopulmonary support machine developed by the Department of Cardiothoracic Surgery, Skane University Hospital, Scania, Sweden and Lund University, both addressed above in section 2, entitled Background of the Invention, subsection a. General.

As indicated, when a single expandable confluence conduit cannot span the distance separating the donor and recipient, the longer, or as shown, the upper of the two lines to be discarded is made of ordinary tubing. To allow growth when implanted in an infant, the lower, shorter line then comprises an expandable fabric confluence conduit described below in section 2b(2)(f), entitled Expandable Structure to Accommodate Growth and Self-adjust in Size in Vascular Segment Prostheses, Lines, and Confluence Conduits Placed in Children, shown in FIGS. 15C, 15D, 36A, and 36F. Implantation requires switching from a longer to a shorter line, and accommodating growth with an expandable shorter line. Past adolescence, it may be necessary to reinsert and switch back to a longer line, preferably accomplished endoscopically whenever possible. FIG. 35B shows line or confluence conduit long to short switchable confluence conduit.

Both the longer line and the shorter conduit to which the longer line is switched before implantation provide the function of a confluence conduit, such as connection between a different number of like vessels to eliminate the need for time-consuming anastomoses and the release of medication into the confluent line. As shown in FIGS. 32D thru 32F, when implanted, the bloodline switches are removed along with the conduit. Since use of expandable fabric in the confluence conduit and bypasses allows for growth, a need to reoperate in order to switch back to a longer line will not arise.

FIGS. 32A thru 32C show a single bloodline long-to-short or short-to-long switch which allows changing the length of a bloodline without interruption in the flow of blood, and FIG. 35B shows a confluence conduit long-to-short or short-to-long switch which likewise allows changing the length of the confluence conduit connecting several bloodlines without interruption in the flow of blood. The shorter of the lines of the confluence conduit can be implanted but this must be done with the input and output bloodline switches remaining connected.

To allow expansion for growth, all bloodlines and the lower shorter conduit are made of the expansion configured fabric. A long to short or the reverse switchable confluence conduit adds the advantages of line length switchability to the advantages of a confluence conduit such as avoiding the need for intricate and time-consuming anastomoses where the need for differential medication of the anastomotic segments is not a consideration. Since the anastomotic segments can be targeted through the servovalve druglines or accessory channels, removal of the conduit as shown in FIG. 35F is to eliminate its bulk if implanted.

Use of the expandable fabric to produce standardized thoracic aortic arch and other major vessel replacement prostheses is addressed below in section 2b(2)(c)(c2), entitled Automatic Insertion into a Ductus of an Inline Coupling Jacket-secured Bridging, or Tie-line and Its Replacement if Necessary, and shown in FIGS. 37A thru 37F. FIG. 35B shows a confluent long to short or the reverse switchable bloodline which rather than pertinent to a single bloodline, or mainline, as shown in FIGS. 32A thru 32C allows the connection of multiple corresponding arteries or veins between the recipient and donor, eliminating the need to connect each such blood supply and drainage-corresponding pair—especially where anomalous accessory vessels are present—directly through a separate origin to destination vessel-addressed bloodline.

In this way, the use of a confluence conduit expedites solid organ, especially heart transplantation, using the switch, or compound vessel bypass technique, not only by reducing the number of separate long to short bloodlines connecting each corresponding pair of vessels between recipient and donor each of which will then require the separate removal or insertion of the upper long line, but provided the same combination of corresponding vessels are connected to the inlet and outlet of the confluent line, eliminates the need to trace separate bloodlines from origin to destination in order to confirm the correspondence of each pair.

Significantly, when implanted in a frail neonate, the use of expandable switchable bloodlines in switchable confluence conduits to accommodate growth following a heart transplant should reduce the need for open revision to no more than a single instance, and that after a considerable interval. When necessary to cover the distance from donor to recipient, implantation of the short expandable arterial and venous confluent conduits along with their input and output bloodlines correspond to spanning the distance involved with long confluent conduits, if necessary, including inline flow-through ventricular assist type pumps, if necessary, part number 167 as shown in FIGS. 32A and 32B.

2b(1)(b)(b16). Direct Aortocaval Connection: Unified Entry Arterial and Exit Venous Confluence Conduits for the Heterotopic Connection of a Graft Heart in the Lower Abdomen

Aortocaval connection of a graft heart and such connection as synonymous with placement in the lower abdomen are both exemplary: aortocaval connection can be used to perfuse a graft organ positioned elsewhere in the body, and an organ other than a heart can be placed. Placement of a second heart in the abdomen may require the removal of a short segment from the gut, no more than about one foot long, which carefully anastomosed and kept sterile should heal without any effect upon digestion.

An important caveat for placement of a graft organ in the abdomen is to position the organ well clear of the liver. Due to its regenerative function, resection of even one lobe of the liver will result in failure. Bloodlines that traverse the waist and pose the risk for bending to a degree that would impair normal pulsatile blood flow much less kink to cut off blood flow entirely should be placed with backup bloodlines as addressed above in section 2a, entitled Background of the Invention. General, in the subsection entitled Backup Path to Protect against Flow Obstruction of Bloodlines Traversing the Waist following Compound Heterotopic Bypass Transplantation of a Second Heart in the Lower Abdomen

An expedient employed by researchers studying the immunological factors in graft organ rejection rather than physiological and surgical considerations no less critical for graft function and survival is to connect the harvested graft organ, a kidney, in the lower abdomen directly to the abdominal aorta and the inferior vena cava. However, blood flow through some vessels reduced and graft organ vasculature endothelium requiring to be constantly washed over by the passage of blood, this has been found to result in endothelial dysfunction and disuse atrophy, which akin to the ischemia following organ harvesting, can result in reperfusion injury with an increased likelihood of graft failure.

To avoid disuse atrophy when the graft organ is position in the lower abdomen and anastomosed directly to the abdominal aorta and inferior vena cava so that not all the organ vasculature is perfused, a unified entry confluence conduit as described in this section is used. Unified entry confluence conduits have a single connecting bloodline for connection to either the abdominal aorta or to the inferior vena cava at one end, the opposite end providing 9 connecting bloodlines for connection to each artery in a direct aortocaval connection.

Disuse atrophy is avoided when the graft organ, especially when a second heart with its many vessels, is positioned in the lower abdomen as one possibility enunciated above in section 2b(1)(b)(b5), entitled Heterotopic Double Heart Metered Compound Vascular Bypass, or Switched-organ Transplantation [and so on]. Positioned thus with every vessel connected vessel to vessel with the native or earlier transplanted heart in the chest. And provided the added heart is large and robust enough, connection directly to the abdominal aorta and inferior vena cava is probably unnecessary, but is available as an option to assist in overcoming the resistance to gravity that would be the case in a connection to the native heart in the chest alone.

Perfusion of all the arteries is made possible through the use of a unified entry arterial and exit venous confluence conduits as addressed above in section 2b(1)(b)(b5), entitled Heterotopic Double Heart Metered Compound Vascular Bypass, or Switched-organ Transplantation and the Use of Unified Entry Arterial and Venous Confluence Conduits to Avoid Disuse Atrophy when the Graft Organ is Positioned in the Lower Abdomen by Direct Aortocaval Connection, as well as in this section. In a compound vascular bypass, or switched-organ direct aortocaval connection heart transplant, the second heart while remaining in the donor having been placed on life support prior to having expired is never connected directly to the native or earlier replaced heart of the recipient.

Instead, the oxygenated blood-conveying vessels of the donor heart are connected to the abdominal aorta of the recipient, while the unoxygenated blood-conveying vessels are connected to the inferior vena cava of the recipient. While placing the recipient between two deceased donors to either side would allow one accessory heart to be transplanted by heterotopic double-heart transplantation and a second accessory heart by direct aortocaval connection, only the most severe lack of adequate replacement hearts and exigent condition of the patient would warrant such a procedure.

The concept of heterotopically positioning a graft heart in the abdomen in itself anything but novel; however, not having used vascular servovalves but rather conventional surgical anastomoses, connections were not accomplished without interrupting blood flow and ischemia, the rate of flow through the junctions could not be modulated, nor medication directly pipe-targeted to the treatment site much less automatically in response by sensor inputs fed to the implanted disorder response system master control microprocessor. These factors fundamentally change the ‘playing field.’

No less importantly, from the present point of view which seeks means for treating patients with end stage heart disease and neonates with unsurvivable malformations of the heart, the xenotransplantation factor introduced a basic immunological complication prone to result in hyperacute rejection (Dun, H., Ye., L., Zhu, Y., and Wong, B. W. 2020. “Combined Abdominal Heterotopic Heart and Aorta Transplant Model in Mice,” Online, PLoS [Public Library of Science] One 15(6):e0230649; Goerlich, C. E., DiChiacchio, L., Zhang, T., Singh, A. K., Lewis, B., and 7 others 2020. “Heterotopic Porcine Cardiac Xenotransplantation in the Intra-abdominal Position in a Non-human Primate Model,” Online, Nature Research Scientific Reports 10(1):10709; Sliwka, J. E., Tyrpien, M., Wilczek, P. M., Zembala, M., and Przybylowski, P. 2020. “A Modified Heterotopic Heart Transplantation in the Rat—as an Important Model in Experimental Regeneration and Replacement of the Failing Organ,” Online, Kardiochirurgia i Tokakochirurgia Polska (Polish Journal of Cardiothoracic Surgery) 17(3):149-154; DiChiacchio, L., Singh, A. K., Chan, J. L., Shockcor, N. M., Zhang, T., and 5 others 2019. “Intra-abdominal Heterotopic Cardiac Xenotransplantation: Pearls and Pitfalls,” Online, Frontiers in Cardiovascular Medicine 6:95; Narayan, P. 2017. “Soon, Patients May Get a Heart Transplant in Stomach to Help Them Survive Weak Heart,” at Indiatimes.com (Times of India) 10 December 17; Seth, S. 2017. “Heart Transplant in the Abdomen,” Journal of the Practice of Cardiovascular Sciences 3(3):132; Liu, F. and Kang, S. M. 2007. “Heterotopic Heart Transplantation in Mice,” Online, Journal of Visualized Experiments (6):238; Caparrelli, D. J., Shake, J. G., Cattaneo, S. M. 2nd, Chang, B. Y., Baumgartner, W. A., Conte, J. V. Jr., and George, P. 2002. “Heterotopic Abdominal Heart Transplantation: A Pilot Study,” Current Opinion in Cardiology 17(2):145-151; Ohmi, M., Yokoyama, H., Nakame, T., Murata, S. Akimoto, H., Tabayashi, K., and Mohri, H. 1992. “Hemodynamic Performance in a Heterotopically Transplanted Dog Heart: Proposal of Techniques for Working Left Heart Model of Heterotopic (Abdominal) Heart Transplantation,” Journal of Heart and Lung Transplantation (New York, New York) 11(6):1147-1150; Klein, I., Hong, C., and Schreiber, I. S. 1991. “Isovolumic Loading Prevents Atrophy of the Heterotopically Transplanted Rat Heart,” Online, Circulation Research 69(5):1421-1425).

The seamless and ischemia-free transfer of the heart from the circulatory system of the donor sustained on life support past death and into the circulatory system of the recipient with direct aortocaval connection is accomplished through unified entry arterial and exit venous confluence conduits addressed above in section 2b(1)(b)(b5), entitled Heterotopic Double Heart Metered Compound Vascular Bypass, or Switched-organ Transplantation and the Use of Unified Entry Arterial and Venous Confluence Conduits.

Each valve at the ends of either unified entry arterial and venous confluence conduit is equipped with at least one drugline, part number 8 or 8′ in the drawing figures, the ability to program the implanted automatic prosthetic disorder response system to directly pipe-target a sensor-detected need for or scheduled immunosuppressive, anticoagulant, antimicrobial, and/or anti-inflammatory is unlimited in target selectivity. Sensors are addressed above in section 2a, entitled Background. General.

Used to connect the oxygen carrying vessels to include the thoracic aorta and pulmonary veins of the graft to the abdominal aorta and the deoxygenated to include the superior vena cava and the pulmonary artery to the inferior vena cava of the recipient, these generally have only one large bloodline and vascular valve for emergency use or a servovalve for nonemergency connection to the abdominal aorta and inferior vena cava of the recipient.

If the distance between the donor and recipient permits, these conduits are made of the expandable fabric described below in section 2b(2)(f), entitled Expandable Structure to Accommodate Growth and Self-adjust in Size in Vascular Segment Prostheses, Lines, and Confluence Conduits Placed in Children. This both allows avoiding the more complicated use of switchable confluence conduits as described above in section 2b(1)(b)(b15), entitled Switchable Lines and Confluence Conduits . . . and makes it possible to elevate the heart connected thus up into the chest without further disconnections and connections.

The opposite end of the conduits provides a bloodline with valve for each vessel of the donor heart. To induce a measure of immune tolerance induction through microchimerization inherent in the transfer procedure whereby the relative proportion of the blood flowing through the graft is more and more that of the recipient, the transfer from the circulatory system of the donor into that of the recipient is best accomplished gradually through vascular servovalves under the control of the prosthetic disorder response system. Connection thus is unlike that described in the intra-abdominal heart transplants cited just above in this section.

Circumstances such as disease, surgical resection, or injury can make positioning of the graft organ anywhere but in the abdomen inadvisable if not impossible. In such a case, a repositioning of a single replacement heart heterotopically thus might be justified. There are, then, both intrinsic, or physiological, and the possibility of exigent considerations that justify such a departure from the norm. A heterotopic second heart positioned in the peritoneal cavity, in the lower abdomen that is, through a local aortocaval rather than a vessel-to-vessel connection is shown schematically without a heart cage for clarity in FIG. 17B. Arterial and venous confluence conduits are the same device with that arterial facing in the opposite direction as that venous.

The absolute number of arterial or venous branches at the end of the conduit opposite to the entry depends upon whether—as will usually be the case—the organ is a heart or a kidney, and if a kidney, the number of vessels is normal, that is, without accessory or supernumerary vessels. As does FIG. 15A, further to eliminate needless complexity, FIG. 17B combines, or summarizes, arteries and veins. The flow pattern at the upper and lower left in FIG. 17B is the same as it is in FIG. 15A. Notwithstanding the concurrent reduction in pressure in the venous return at the lower left, the increase in pressure due to the convergence of the donor and recipient arterial lines at the upper left is continued only during transfer of the organ and affects vasculature that will be omitted from the transfer.

Nevertheless, as addressed next, the system provides means for relieving the ephemeral increase in pressure should a need arise. As shown in FIG. 17B, aortocaval connection provides perfusion through all the vessels of the graft organ thus dispelling disuse atrophy, however, since return flow from the graft to the aorta of the recipient is lacking, the rate of microchimerization is somewhat less, slightly extending the duration of metered reciprocal cross-circulation.

In these regards, direct aortocaval connection, because it involves no connection of the graft to the native organ, offers some potential advantages over a switched-organ heterotopic double heart transplant in the chest as described above in section 2b(1)(b)(b5), entitled Heterotopic Double Heart Metered Compound Vascular Bypass, or Switched-organ Transplantation and the Use of a Unified Entry Confluence Conduit to Avoid Disuse Atrophy when the Graft Organ is Positioned in the Lower Abdomen by Direct Aortocaval Connection.

Unlike a ventricular assist device, the heterotopic positioning of a second heart, for example, to act as a natural booster to assist an impaired native heart will:
1. As organic rather than mechanical, support the metabolic function and coordination of the body.
2. Provide pulsatile rather than continuous flow, the latter having been established as harmful over time.
3. Essentially halving the number of recipient arterial and donor venous servovalves, thereby reducing both the procedural duration and the cost.
4. Eliminate the resistance of gravity to venous return from the lower abdomen back up to the thorax.
5. Avoid the high cost of mechanical assist devices which makes these unobtainable throughout much of the world.
6. Will sustain perfusion through all of the vasculature of the graft organ, thereby supporting endothelial function and precluding disuse atrophy.
7. If placed in a child, the graft will grow in step with the rest of the body, thus eliminating the need for at least one revision through a painful and risk-ridden midsternal, clamshell, or herniclamshell thoracotomy at adolescence or early adulthood.
8. Rather than for the arterial and venous flow of the second heart to pass through as to depend upon the proper functioning of that native and its servovalves, each heart is:
1. Differently connected and therefore independently serviceable, with either
2. Eventually removable without dealing with multiple connecting bloodlines, where each
3. Can be directly pipe-targeted medication, and without the added heart
4. Posing the contingency of even a brief interval of ventricular hypovolemia as would prompt the need for toggling.

Related sections herein are:
a. Heterotopic double heart transplantation is addressed above in section 2b(1)(b)(b5), entitled Heterotopic Double Heart Metered Compound Vascular Bypass, or Switched-organ, Transplantation [and so on), and unified entry confluence conduits for the heterotopic positioning of a graft organ in the lower abdomen is addressed above in section 2b(1)(b)(b7), entitled Metered compound vascular bypass, or switched-organ liver or heart-liver transplantation b. Confluence conduits are addressed above in section 2b(1)(b)(b14), entitled Confluence Conduits: Connection of One or More Bloodlines to Flow through a Common Channel to the Same or a Different Number of Bloodlines with Self-leveling Apportionment of Outflow.
c. Switchable confluence conduits when necessary to eliminate the slack of long bloodlines needed to extend between donor and recipient are addressed above in section 2b(1)(b)(b15), entitled Switchable Lines and Confluence Conduits: Long When Intercorporeal, to Short When Intracorporeal, Both Affording Connection of One or More Bloodlines to the Same or a Different Number of Bloodlines with Self-leveling Apportionment of Outflow.
d. Direct aortocaval connection as addressed above in section 2b(1)(b)(b5), entitled Heterotopic Double Heart Metered Compound Vascular Bypass, or Switched-organ Transplantation, and the Use of a Unified Entry Confluence Conduit to Avoid Disuse Atrophy when the Graft Organ is Positioned in the Lower Abdomen by Direct Aortocaval Connection.
e. A general introduction to aortocaval connection is presented in this section.
f. A structural description of unified entry aortocaval confluence conduits is provided below in section 6, entitled Description of the Preferred Embodiments of the Invention.

In a metered organ transplant, the relative proportion of recipient to donor arterial blood passed to the graft organ is gradually increased until the graft has effectively been switched from the circulatory system of the donor into the circulatory system of the recipient. Now to clamp and transect the vessels of the donor outside the level of the blood circuit does not result in an interruption in perfusion. The use of adjuvant immune tolerance induction medication is not discounted.

This gradual transfer between circulatory systems is extended long enough to accomplish an initial measure of immune tolerance, essentially to minimize if not eliminate the shock of a stark confrontation with an alien object, or foreign body. Venous retum flow from the graft organ to the donor is accomplished by means of the same arrangement in reverse—that is, the volume of venous return is controlled by a corresponding servovalve about the inferior vena cava of the recipient where the individual connectors on the veins of the graft consist of one servovalve on each. That is, the venous connection is identical to the arterial but in reverse.

Aortocaval connection of the recipient abdominal aorta to the graft organ in the donor, having been initiated on life support prior to death—to make possible a metered heterotopic double heart transplant, for example, consists of a single vascular servovalve positioned along the abdominal aorta of the recipient, which servovalve is bonded as to be integral with the entry end of an aortocaval confluence conduit. This diverts blood from the recipient aorta which is conveyed into the graft organ through the coordinated action of servovalves on each artery of the graft organ, the apportionment of blood flow no different than as depicted in FIG. 15A. Connection with the inferior vena cava is the same but in reverse.

As pertains to a heterotopic switched organ double heart transplant where the addition of a heart is unaccompanied by the addition of a one or two lungs, provided the native lungs are unaffected, to oxygenate the fraction of unoxygenated blood contributed by the accessory heart whether by vessel-to-vessel connection or by input directly into the inferior vena cava should not materially surpass the unoxygenated blood ordinarily ejected by the previously failing heart alone to tax normal gas exchange capability.

If necessary, the patient should be considered for a heart-lung transplant. Oxygenation of the accessory heart is through the abdominal aorta as if it were an abdominal organ. If a lung must be added, whether vessel-by-vessel or aortocavally connected, the accessory heart is positioned in the lower abdomen and the third lung accommodated as would a second heart to the right of that native as described above in section 2b(1)(b)(b5), entitled Heterotopic Double Heart Metered Compound Vascular Bypass, or Switched-organ Transplantation [and so on].

To create the space for an accessory lung, the lower half of the right lung is removed, which will provoke it to regenerate a higher density of the alveoli and microcirculation within its reduced volume. The same approach to increasing the absolute number of alveoli should be considered whether the directly aortocavally connected heart is retained in the lower abdomen or just connected there and positioned in the chest to the right of the native heart as addressed in the following section on doubly heterotopic double heart transplantation.

Depending upon the state of the art following development since 1970 (Fazzalari, F. L., Bartlett, R. H., Bonnell, M. R., and Montoya, J. P. 1994. “An Intrapleural Lung Prosthesis: Rationale, Design, and Testing,” Artificial Organs 18(11):801-805), the placement of an implantable artificial lung, still limited to short-term use, may be considered to complement the increased pumping capacity contributed by the accessory heart. There remains presently “no truly ambulatory artificial lung” (Batcheldor, M. 2022. “Multicenter Team Seeks to Create at-Home Artificial Lung System,” Online, Vanderbilt University Medical Center Reporter).

The term artificial ‘lung’ is often used for nonimplantable wearable breathing assist devices. Once successful, such a device can be integrated into the control regimen of the implanted disorder response system. A review of attempts to devise an ambulatory, or implantable, artificial lung should expedite continued attempts (see, for example, Duy Nguyen, B. T., Nguyen, Thi, H. Y., Nguyen Thi, B. P, Kang, D. K., and Kim, J. F 2021. “The Roles of Membrane Technology in Artificial Organs: Current Challenges and Perspectives, Online, MDPI [Multidisciplinary Digital Publishing Institute] Membranes (Basel, Switzerland) 11(4):239; Syed, A. Kerdi, S., and Qamar, A. 2021. “Bioengineering Progress in Lung Assist Devices,” Online, Bioengineering (Basel, Switzerland) 8(7):89; Arens, J. Grottke, O., Haverich, A., Maier, L. S., Schmitz-Rode, T., and 3 others 2020. “Toward a Long-term Artificial Lung,” Online, American Society for Artificial Internal Organs Journal 66(8):847-854; Hopkins, S. R. 2020. “Ventilation/Perfusion Relationships and Gas Exchange: Measurement Approaches,” Online, Comprehensive Physiology 10(3):1155-1205; Swol, J., Shigemura, N., Ichiba, S., Steinseifer, U., Anraku, M., and Lorusso, R. 2020. “Artificial Lungs—Where are We Going with the Lung Replacement Therapy?,” Artificial Organs 44(11):1135-1149; Madhani, S. P., Frankowski, B. J., Ye. S. H., Burgreen, G. W., Wagner, W. R., and 3 others 2019. “In Vivo 5 Day Animal Studies of a Compact, Wearable Pumping Artificial Lung,” Online, American Society for Artificial Internal Organs Journal 65(1):94-100; Orizondo, R. A., Cardounel, A. J., Kormos. R., and Sanchez, P. G. 2019. “Artificial Lungs: Current Status and Future Directions,” Online, Current Transplantation Reports 6:307-315; Naito, N., Cook, K., Toyoda. Y., and Shigemura, N. 2018. “Artificial Lungs for Lung Failure: JACC Technology Corner,” Online, Journal of the American College of Cardiology 72(14):1640-1652; Madhani, S. P., Frankowski, B. J., Burgreen, G. W., Antaki, J. F., Kormos, R., D'Cunha, J., and Federspiel, W. J. 2017. “In Vitro and in Vivo Evaluation of a Novel Integrated Wearable Artificial Lung,” Online, Journal of Heart and Lung Transplantation (New York, New York) 36(7): 806-811; Alghanem, F., Davis, R. P., Bryner, B. S., Hoffman, H. R., Trahanas, J., and 4 others 2015. “The Implantable Pediatric Artificial Lung: Interim Report on the Development of an End-stage Lung Failure Model,” Online, American Society for Artificial Internal Organs Journal 61(4):453-458; Strueber, M. 2015. “Artificial Lungs: Are We There Yet?,” Thoracic Surgery Clinics 25(1):107-113; Ye, S. H., Arazawa, D. T., Zhu, Y., Shankarraman, V., Malkin, A. D., and 5 others 2015. “Hollow Fiber Membrane Modification with Functional Zwitterionic Macromolecules for Improved Thromboresistance in Artificial Lungs,” Online, American Chemical Society Langmuir 31(8):2463-2471; Martin, J. T. and Zwischenberger, J. B. 2013. “Artificial Lung and Novel Devices for Respiratory Support,” Seminars in Thoracic and Cardiovascular Surgery 25(1):70-75; Schewe, R. E., Khanafer, K. M., Arab, A., Mitchell, J. A., Skoog. D. J., and Cook, K. E., 2012. “Design and in Vitro Assessment of an Improved, Low- resistance Compliant Thoracic Artificial Lung,” Online, American Society for Artificial Internal Organs Journal 58(6):583-589; Nolan, H., Wang, D., and Zwischenberger, J. B. 2011. “Artificial Lung Basics: Fundamental Challenges, Alternative Designs, and Future Innovations,” Online, Organogenesis 7(1):23-27; Ota, K., 2010. “Advances in Artificial Lungs,” Journal of Artificial Organs 13(1):13-16; Kaar, J. L., Oh, H. I., Russell, A. J., and Federspiel. W. J. 2007. “Towards Improved Artificial Lungs through Biocatalysis,” Online, Biomaterials 28(20):3131-3139; Tatsumi, E. 2007. “Artificial Lungs: Current State and Trends of Clinical Use and Research and Development,” Journal of Artificial Organs 10(1) 1-5; Sato, H., Taga, I., Kinoshita, T., Funakubo, A, Ichiba, S., and Shimizu, N. 2006. “In Vitro Evaluation of a Newly Developed Implantable Artificial Lung,” Online, Acta Medica Okayama 60(2):113-119; Zwischenberger, B. A., Clemson, L. A., and Zwischenberger, J. B. 2006. “Artificial Lung: Progress and Prototypes,” Expert Review of Medical Devices 3(4):485-497; Cook, K. E., Perlman, C. E., Seipelt. R., Backer, C. L., Mavroudis. C., and Mockrost, L. F. 2005. “Hemodynamic and Gas Transfer Properties of a Compliant Thoracic Artificial Lung,” Online, American Society for Artificial Internal Organs Journal 51(4):404-411: Taga. I., Funakubo, A., and Fukui, Y. 2005. “Design and Development of an Artificial Implantable Lung Using Multiobjective Genetic Algorithm: Evaluation of Gas Exchange Performance,” Online, American Society for Artificial Internal Organs Journal 51(1):92-102; Lick, S. D. and Zwischenberger, J. B. 2004. Artificial Lung: Bench toward Bedside,” Online, American Society for Artificial Internal Organs Journal 50(1):2-5; Zwischenberger, J. B. 2004. “Future of Artificial Lungs,” Online, American Society for Artificial Internal Organs Journal 50(6):xlix-li; Zwischenberger, J. B. and Alpard, S. K. 2002. “Artificial Lungs: A New Inspiration,” Perfusion, 17(4):253-268; Zwischenberger, J. B., Anderson, C. M., Cook, K. E., Lick, S. D., Mockros, L. F., and Bartlett, R. H. 2001. “Development of an Implantable Artificial Lung: Challenges and Progress,” Online, American Society for Artificial Internal Organs Journal 47(4):316-320; Cook, K. E., Makarewicx, A. J., Backer, C. L., Mockros, L. F., Przybylo, H. J., and 4 others 1996. “Testing of an Intrathoracic Artificial Lung in a Pig Model,” American Society for Artificial Internal Organs Journal 42(5):M604- M609; Fazzalari, F. L., Bartlett, R. H., Bonnell, M. R., and Montoya, J. P. 1994, Op cit., above in this paragraph; Fazzalari, F. L., Montoya, J. P., Bonnell, M. R., Bliss, D. W., Hirschl. R. B., and Bartlett, R. H. 1994. “The Development of an Implantable Artificial Lung,” American Society for Artificial Internal Organs Journal 40(3):M728-M731; Hattler, B. G., Reeder, G. D., Sawzik, P. J., Lund, L. W., Walters, F. R., and 5 others 1994. “Development of an Intravenous Membrane Oxygenator: Enhanced Intravenous Gas Exchange through Convective Mixing of Blood around Hollow Fiber Membranes,” Artificial Organs 18(11):806-812; Hattler, B. G., Reeder, G. D., Sawzik, P. J. Walters. F. R., Pham, S. M. and 5 others, et al. 1994. “Development of an Intravenous Membrane Oxygenator: A New Concept in Mechanical Support for the Failing Lung,” Journal of Heart and Lung Transplantation (New York, New York) 13(6):1003-1008; Vaslef, S. N., Cook, K. E., Leonard, R. J., Mockros, L. F., and Anderson, R. W. 1994. “Design and Evaluation of a New, Low Pressure Loss, Implantable Artificial Lung,” American Society for Artificial Internal Organs Journal 40(3):M522-M526; Vaslef. S. N., Mockros, L. F., Cook, K. E., Leonard, R. J., Sung, J. C., and Anerson, R. W. 1994. “Computer-assisted Design of an Implantable, Intrathoracic Artificial Lung,” Artificial Organs 18(11):813-817; Morin, P. J., Gosselin, C., Picard, R., Vincent. M., Guidon, R., and Nicholl, C. I. 1977. “Implantable Artificial Lung. Preliminary Report,” Journal of Thoracic and Cardiovascular Surgery 74(1):130-136; Palmer, A. S., Collins, J., and Head. L. R. 1973. “Development of an Implantable Artificial Lung,” Journal of Thoracic and Cardiovascular Surgery 66(4):521-525; Palmer, A., Collins, J., Bodell, B., and Head, L. R. 1972. “An Implantable Artificial Lung,” Surgical Forum 23(0):231-233; Shah-Mirany, J., Head, L. R., Ghetzler, R., Formolo, A. J., Palmer, A. S. and Bodell, B. R. 1972. “An Implantable Artificial Lung,” Annals of Thoracic Surgery 13(4):381-387).

Improvements in extracorporeal membrane oxygenation and wearable artificial ‘lungs’ are significant in developing new gas exchange membranes suitable for improvement in extracorporeal membrane oxygenators, but do not offer the reduction in size and implantability needed in an artificial lung (see, for example, Fukuda, M. 2023. “Evolutions of Extracorporeal Membrane Oxygenator (ECMO): Perspectives for Advanced Hollow Fiber Membrane,” Online, Journal of Artificial Organs (Tokyo, Japan) 14:1-6; Wang, Y., Liu, Y., Han, Q., Lin. H., and Liu, F. 2022. “A Novel Poly(4-methyl-1-pentene)/Polypropylene (PMP/PP) Thin Film Composite (TFC) Artificial Lung Membrane for Enhanced Gas Transport and Excellent Hemno-compatibility,” Online, Journal of Membrane Science 5:649120359; Farling, S. 2021. Development of a Hyperbaric Intravascular Membrane Oxygenator Catheter, Dissertation, Department of Civil and Environmental Engineering, Duke University; Orizondo, R. A., Omecinski, K. S., May, A G., Dhamotharan, V., Frankowski, B. J., and 7 others 2021. “Month-long Respiratory Support by a Wearable Pumping Artificial Lung in an Oine Model,” Online, Transplantation 105(5):999-1007; Fukuda, M., Tokumine, A., Noda, K., and Sakai, K. 2020. “Newly Developed Pediatric Membrane Oxygenator that Suppresses Excessive Pressure Drop in Cardiopulmonary Bypass and Extracorporeal Membrane Oxygenation (ECMO),” Online, MDPI [Multidisciplinary Digital Publishing Institute] Membranes (Basel, Switzerland) 10(11):362).

If and only if troublesome, the gradual increase in hypertension in each donor artery as the diversion chute of its servovalve moves adaxially to allow more recipient aortic blood to enter from the aorta of the recipient while obstructing the passage of native arterial blood (at the upper left in FIG. 15A) can be decompressed by tapping off the excess volume of blood and passing it to a large neighboring vessel by means of an ancillary servovalve. Concern for the condition of the vasculature up- and downstream to the graft organ may be justified only when the deceased donor is to be further maintained for the harvesting of other organs or tissues.

The same applies at the venous return where the blood from the recipient is added to that native (at the lower left in FIG. 15A). Such pressure relief, or decompression, vascular servovalves up- and downstream to the primary, or graft transfer, servovalves are placed under the same control as the primary and advanced in the same proportion. Means for accomplishing localized pressure relief are addressed below in section 2b(2)(h)(h4), entitled The ‘Tuned’ Shunt in the Relief of Localized Vascular Hypertension. The procedure concluded, such ancillary servovalves used for midprocedural pressure relief are recovered and sterilized for reuse.

If continued use of the donor as a source of replacement organs is to follow, the arterial and venous stumps are directly anastomosed and dialysis and/or apheresis used to protect the blood against what would otherwise be a rapid buildup in toxicity that would end the usability of the donor as a source of graft organs and tissues. Preservation of the deceased donor is addressed above in section 1a, entitled Introduction. General, and involves training in the use of extracorporeal membrane oxygenation, ventilation and/or the use of a surgical chest dome as described below in section 2b(2)(k), entitled Surgical Chest Dome, and shown in FIGS. 23A thru 23E, the maintenance of temperature, endocrine, and nutritional function, as well as equipment to forestall toxicity and decomposition.

To expand the pool and expedite the availability of replacement organs and tissues without the deterioration and increase in the probability of post-implantation graft failure and rejection due to ischemia and reperfusion injury, large centers should maintain an in-house department entrusted with the preservation of suitable deceased patients who had volunteered to serve as organ donors.

2b(1)(b)(b17). Doubly Heterotopic Double Heart Compound Vascular Bypass, or Switched-Organ Transplantation

The difference between a doubly heterotopic double heart compound vascular bypass, or switched-organ heart transplant and an ordinary compound vascular bypass heterotopic heart transplant as addressed above in section 2b(1)(b)(b5), entitled Heterotopic Double Heart Metered Compound Vascular Bypass, or Switched-organ Transplantation [and so on] is that while the graft organ in the latter can be positioned outside the chest, all the connections between the donor and recipient hearts are directly between the two hearts. Bloodlines that traverse the waist and pose the risk for bending to a degree that would impair normal pulsatile blood flow much less kink to cut off blood flow entirely should be placed with backup bloodlines as addressed above in section 2a, entitled Background of the Invention in the subsection entitled Backup Path to Protect against Flow Obstruction of Bloodlines Traversing the Waist following Compound Heterotopic Bypass Transplantation of a Second Heart in the Lower Abdomen.

Aortocavally connected transplants in the abdomen and connected to the native or earlier transplanted heart or repositioned in the chest and then connected to the native or earlier transplanted heart are connected to each of the vessels of the graft organ but not between the donor and recipient hearts. Accordingly, these chest-to-abdomen transplants are not reciprocal, convergent, of interchangeable. However, once both are in the chest, connections directly between the two hearts are readily possible and as addressed above in section 2a, entitled Background. General, probably advantageous.

In a doubly heterotopic double heart compound vascular bypass heart transplant, the graft heart is not connected directly as in a direct aortocavally connected transplant to the abdominal aorta and inferior vena cava of the recipient but rather through an elongated confluence conduit which allows the graft organ to be positioned in the chest to the right of the native heart as shown in FIG. 17A (Ji, Z., Liu, Y., and Wang, L. 2019. “Abdomen is the Second “Home” of the Heart,” (in Chinese with English abstract at Pubmed) Zhonghua Wei Zhong Bing Ji Jiu Yi Xue [Critical and Intensive Care Medicine] (Beijing, China) 31(4): 390-392). Connected thus, the added heart is dependent upon the native heart to pass through its arterial supply and venous drainage. In a hybrid doubly heterotopic double heart transplant addressed below in section 2b(1)(b)(b18), entitled Hybrid Doubly Heterotopic Double Heart Transplantation, the aorta and pulmonary vessels are connected directly between the two hearts across the chest.

That is, in a direct aortocaval vascular bypass, or switched-organ double heart transplant as described in the preceding section, the connection to the arteries of the unharvested donor, or graft organ, is through a unified entry arterial confluence conduit as described above in the preceding section, 2b(1)(b)(b16), entitled Direct Aortocaval Connection: Unified Entry Arterial and Exit Venous Confluence Conduits for the Heterotopic Connection of a Graft Heart in the Lower Abdomen, that is, connection is directly to the abdominal aorta of the recipient, and connection of recipient veins is through a unified entry venous confluence conduit connected directly to the inferior vena cava of the recipient, the graft organ then positioned in the lower abdomen.

The provision of adequate space to the right of the native or previously compound vascular bypass orthotopically transplanted heart is addressed above in section 2b(1)(b)(b5), entitled Heterotopic Double Heart Metered Compound Vascular Bypass, or Switched-organ Transplantation [and so on]. Positioning the aortocavally connected heart in the chest requires unified entry arterial and venous confluence conduits that are otherwise ordinary but exceptionally equipped with longer aortic and vena caval bloodlines to reach up to the chest. The sections of either that pass, or subtend, the waist and must flex without flattening or kinking are made of a tougher material similar to auto coolant hosing, as described above in section 1a. Background. General and in section 6, entitled Description of the Preferred Embodiments of the Invention.

Unified entry arterial and venous confluence conduits are described above in section 2b(1)(b)(b5), entitled Heterotopic Double Heart Metered Compound Vascular Bypass, or Switched-organ Transplantation and the Use of Unified Entry Arterial and Venous Confluence Conduits to Avoid Disuse Atrophy when the Graft Organ is Positioned in the Lower Abdomen by Direct Aortocaval Connection. By contrast, in a compound vascular bypass, or switched-organ double heart transplant, the diversion of recipient blood is to and through the donor graft organ on a corresponding artery-to-artery and vein-to-vein basis while it remains unharvested in the donor continued on life support after having expired. The advantages in direct aortocaval connection with the graft organ remaining in the lower abdomen are enumerated in the preceding section.

To allow placement thus, the recipient must have lungs sufficiently competent to allow the removal of a segment from the right lung large enough to allow placement of the second heart. Thereafter, the right lung would regenerate in the manner unique to lungs, that is, through an increase in the density of the alveoli within the reduced volume rather than by recovery to its original size and shape as would the liver or the original shape as would a bursa. Unified entry conduits for this purpose are of sufficient length and will usually include an inline pump to facilitate the movement of blood against the resistance of gravity.

In an infant or a small child, the unified entry arterial and venous confluence conduits are made of the seamless expandable fabric devised to allow growth without the need for revision described below in section 2b(2)(f), entitled Expandable Structure to Accommodate Growth and Self-adjust in Size in Vascular Segment Prostheses, Lines, and Confluence Conduits Placed in Children. Even when directly adjacent to the right of the native or earlier orthotopically transplanted heart in the chest, a doubly heterotopic heart offers some advantages in being relatively independent in function. Relatively, because the second heart is no less subject to blood borne endocrine and hormonal indicia comprehensive of a primary heart. The second heart no less supported by servovalves incorporating accessory channels, this greater independence simplifies and expedites treatment both medicinally and surgically, to include the removal and/or replacement of either heart.

2b(1)(b)(b18). Hybrid Doubly Heterotopic Double Heart Transplantation.

In a standard vessel-to-vessel double heart transplant as described above in section 2b(1)(b)(b5), entitled Heterotopic Double Heart Metered Compound Vascular Bypass, or Switched-organ Transplantation [and so on], connection between the vessels of the donor heart is directly to the corresponding vessels of the native or earlier transplanted heart vessel-to vessel across the chest. Placement in the abdomen when all vessels are connected between the donor and recipient vessel-to-vessel is not referred to as doubly heterotopic, which term is reserved for direct aortocaval connection with placement of the graft in the chest without connection to the native or earlier transplanted heart.

Bloodlines that traverse the waist and pose the risk for bending to a degree that would impair normal pulsatile blood flow much less kink to cut off blood flow entirely should be placed with backup bloodlines as addressed above in section 2a, entitled Background of the Invention in the subsection entitled Backup Path to Protect against Flow Obstruction of Bloodlines Traversing the Waist following Compound Heterotopic Bypass Transplantation of a Second Heart in the Lower Abdomen. In a direct aortocaval transplant, connection of the vessels is of those conveying oxygenated blood to the abdominal aorta and those conveying unoxygenated blood to the inferior vena cava. Connection thus with placement of the graft in the chest to the right of the native or earlier transplanted heart is referred to as doubly heterotopic.

In a hybrid doubly heterotopic double heart transplant, the donor heart is connected to the recipient aortocavally but positioned in the chest as in a doubly heterotopic transplant with the exception that the pulmonary vessels are connected directly to those of the native or earlier transplanted heart across the chest for improved efficiency in gas exchange and to preclude the need for inline assist pumps schematically shown as part number 167 in the drawing figures in the thoracic-abdominal bloodlines or confluence conduits.

The term ‘hybrid’ in this connection is unrelated to hybrid surgical procedures as addressed above in section 2a, entitled Background. General or to the use thereof below in section 2b(2)(c), entitled Vascular Segment Prostheses: Inline Coupling Jackets and Bridging, or Tie-lines. To serve both hearts, connection of the pulmonary vessels thus is with the servovalves set to the half open position as schematically depicted in FIG. 15A. Once realized, a long-term implantable artificial lung, addressed above toward the end of section 2b(1)(b)(b16), entitled Direct Aortocaval Connection: Unified Entry Arterial and Exit Venous Confluence Conduits for the Heterotopic Connection of a Graft Heart in the Lower Abdomen, can be positioned near to the accessory second heart in the lower abdomen.

However, note the caveats expressed in section 2b(1)(b)(b16) concerning the placement of a second heart in the lower abdomen given the limited space available in a typical patient to place a heart much less an artificial lung as well. The artificial lungs currently available damage red blood cells and promote clot. Accordingly, such use should be discounted or at most limited to exigent use as a bridge pending recovery of the existing lungs or full functionality of the transplanted lungs (Arens, J. Grottke, O., Haverich, A., Maier, L. S., Schmitz-Rode, T., and 3 others 2020, Op cit. above in section 2b(1)(b)(b16), entitled Direct Aortocaval Connection: Unified Entry Arterial and Exit Venous Confluence Conduits for the Heterotopic Connection of a Graft Heart in the Lower Abdomen

2b(2). The Intracorporeal Diversion of Blood Flow

The term ‘intracorporeal vascular diversion’ refers to applications of vascular valves and servovalves to be implanted within a patient rather than bloodlines passing between an organ recipient and donor during a switch, or compound vascular bypass, organ transplantation, for example. The use of vascular valves, servovalves in particular, adds considerable flexibility to the use of conventional ductus side-entry and nonservovalve-jackets for all types of shunts and bypasses.

By introducing the ability to adjust the cross-sectional area of the passage, hence, the flowrate, through the valve, vascular valves make it possible to vary the flowrate in response to sensor inputs fed to a control microcontroller—or in a more elaborate system, a microprocessor, whereby the system is totally implanted. The potential applications for such an ability are numerous. The term ‘vascular’ as synonymous with ductus to denote urinary diversion by ureteral takeoff is addressed below in section 2c, entitled Vascular Valves and Servovalves in Urinary Diversion.

This dynamic and immediate adjustability, which allows the instant automatic adaptation to the physiological index requiring normalization, is added to that of ordinary ductus side-entry jackets, which provided with one or more accessory channels or sidelines, are able to pipeline target fluid medication directly to the treatment site from small subcutaneously positioned drug reservoirs. Upon receipt by the control processor of an out-of-range input from one or more sensors, the implanted microprocessor open the reservoir outlet and if incorporating a pumped stopcock, to deliver medication to the site and/or another site as the origin of the abnormal value.

An important consideration in specifying and arranging diversion valves in anatomically dense, or tight, areas, such as around the heart, is that bloodlines, or mainlines used to conduct blood, are ideally alike in luminal diameter as the substrate vessel. With large vessels, such as the venae cavae and the aorta, this results in the need for valves which minimized in size notwithstanding, must be obtrusive, that is, too large and too heavy, to assure that the patient will attain comfort once the procedure has been allowed to heal. This problem is alleviated by dividing the flow between two bloodlines leading to the same destination vessel at at an offset, meaning at a separate, usually nearby level.

Bidirectional valves suitable for exchanging flow between two vessels as in an extracardiac repair of a dextro-transposition of the great arteries in a neonate or infant, or diverting flow from one into two different vessels through separate lines, such as do distribute hypertension in the source line by division into two different vessels, another measure for alleviating bulk in this context, where flow is unidirectional, is the use of pull-configured servovalves. Because all ductus side-entry jackets and valves incorporate accessory channels to deliver drugs such as anticoagulants, thrombolytics, and antimicrobials into these devices and the lines these secure, the need for dividing lines as a redundancy measure to protect against obstruction due to clot or crystal, for example, is unnecessary.

To connect donor and recipient venae cavae and aortae for a double heart transplant where the valves used to mediate the transfer from the donor to the recipient will remain implanted rather than replaced or explanted following the operation, the flow can be divided between two lines. To do so not only alleviates the problem of weight and size, but introduces a number of variables which can serve to provide flexibility in the flow overall. Originating from the same source and emptying into the same destination vessel, the second line, similar to a takeoff shunt leading into another vessel placed to relieve hypertension, can be a twin of the first, but can also differ in several respects, to include type ductus side-entry jackets and caliber.

Under the same venous entry pressure or instantaneous arterial entry pressure over the short distances contemplated, the increase in flow rate through a line greater in diameter can usually be discounted. Covering a greater distance, lines that run between the thorax and abdomen or pelvis in an adult move through less congested anatomy than do those within the chest, so that flow through a single wider line with a higher flowrate than a divided line is usually practicable. Such can generally be routed to avoid encroachment upon or by neighboring structures, and should seldom if ever require division.

The bulk of one larger valve is distributed by spacing the divisional valves apart from one another along the substrate vessels. Divisibility thus, into two, exceptionally, even three or more separate smaller valves and bloodlines affords flexibility in accommodating individual peculiarities of patient anatomy, especially in the chest and more still in the chest of a neonate or infant with small and compact anatomy. Smaller valves can be positioned where larger valves cannot, and smaller caliber lines can be passed between structures, such as neighboring vessels and/or nerves or the heart and lungs, for example, where wider lines would encroach upon the surrounding anatomy. All ductus side-entry jackets, valves, and connectors have rounded corners, edges and padding to prevent injury. Changes in diameter along a single line to accommodate anatomy is not preferred.

Ordinarily, flow that would otherwise pass through a single vessel is divided equally between two identical lines, the sum volume of flow for a normotensive source-vessel preserved as divided without a deviation from normotension in the destination vessel. When placed to mediate a metered compound vascular bypass, or sudden switched-organ transplantation, the diversion valves at each end of either line would be servovalves ordinarily adjusted simultaneously and alike. However, in certain situations, such as to avert intracardiac hypovolemia in a double heart transplant, a somewhat increased volume of blood should be provided to the heart not sent the greater volume of blood at the moment during toggling. This can take the form of providing a continuous or intermittent reduction in the volume of flow to either or both of its venae cavae.

2b(2)(a). Transectionless and Ischemia-Free Correction of Complete, or Dextro-Transposition, of the Great Arteries

Much of the problems encountered with transplantation and surgical repair of the defective heart are due to ischemic and general anesthetic injury unavoidable using conventional methods. Transcatheteric or transluminal valvuloplasty and septostomy, for example, avoid cutting into the heart, but not all repairs can be accomplished using such less invasive nonincisional means. Switch transplantation must be open chest, but allows extracardiac, or closed heart, transplantation, with the side effects to unintended tissue of immunosuppressants avoided by directly pipeline targeting these to the site of the graft.

The correction of a transposition is still open chest, but uncomplicated complete d-transposition can be corrected without the need for life support or general anesthesia which reduces if not eliminates the risk of accidental injury to the conduction system and hazardous desynchronization. As in a switched-organ transplant, the numerous critical insults inflicted due to the interruption in perfusion, cardiopulmonary bypass support, cross-clamping, and general anesthesia are eliminated. In many cases, corrective transposition can be repaired by the simultaneous cross-shunting of flow between the vessels.

Other anomalies encountered in transposition taken into account, the operation can be accomplished with or without intracardiac supplementation accomplished transluminally. Shunting has potential applications in other congenital malformities of the heart. Except when due to an intracardiac malformity (see, for example, Alotaibi, F. F., Alotaibi, R. M., Almalki, M. E., Alhassani, M. D., Almuqati, and 3 others 2022. “Patent Foramen Ovale-induced Platypnea-Orthodeoxia Syndrome: A Case Report and Literature Review,” Online, Cureus (Palo Alto, California) 14(12): e32203), some cases allow remediation external to the heart proper.

Extracardiac transposition is ordinarily applicable to congenital malformations external rather than internal to the heart which can result in a failure to isolate unoxygenated, or unsaturated, blood from the right ventricle to the lungs (see, for example, van der Palen, R. L. F., van der Bom, T., Dekker, A., Tsonaka, R., van Geloven, N., and 9 others 2019. “Progression of Aortic Root Dilatation and Aortic Valve Regurgitation after the Arterial Switch Operation,” Online, [British Cardiac Society] Heart (London, England) 105(22): 1732-1740; Joffe, D. C., Shi, M. R., and Welker, C. C. 2018. “Understanding Cardiac Shunts,” Paediatric Anaesthesia 28(4): 316-325; Klein, M. R., Kiefer, T. L., and Velazquez, E. J. 2016. “Platypnea-Orthodeoxia Syndrome: To Shunt or Not to Shunt, that is the Question,” Online, Texas Heart Institute Journal (Houston, Texas) 43(3): 264-266; de Havenon, A., Moore, A., Sultan-Qurraie, A., Majersik, J. J., Stoddard, G., and Tirschwell, D. 2015. “Ischemic Stroke Patients with Active Malignancy or Extracardiac Shunts are More Likely to Have a Right-to-Left Shunt Found by TCD [transcranial Doppler] than Echocardiogram,” Translational Stroke Research 6(5): 361-364; Henkin, S. Negrotto, S., Pollak, P. M., Cullen, M. W., O'Cochlain, D. F., and Wright R. S. 2015. “Platypnea-Orthodeoxia Syndrome: Diagnostic Challenge and the Importance of Heightened Clinical Suspicion,” Online, Texas Heart Institute Journal 42(498-501; Nosib, S., Watt, K., Penz, E., and Fenton, M. 2015. “A 58-year-old Woman with Hypoxia, Hypoxaemia, a Hole in the Heart and a . . . Herring! Intracardiac or Extracardiac Shunt? That is the Question!,” Online, British Medical Journal Case Reports 25 Sep. 2015; Yanna-Schulze, A., Schneider, G., Maßmann, A., Graber, S., and Geisthoff, U. W. 2015. “Carotid Ultrasound for Pulmonary Arteriovenous Malformation Screening,” Open Medicine (Warsaw, Poland) 10(1): 285-293 Stark, R. J. and Shekerdemian, L. S. 2013. “Estimating Intracardiac and Extracardiac Shunting in the Setting of Complex Congenital Heart Disease,” Annals of Pediatric Cardiology 6(2): 145-151; Martinez-Jimenez, S., Heyneman, L. E., McAdams, H. P., Jasinowodolinski, D., Rossi, S. E., Restrepo, C. S., and Washington, L. 2010. “Nonsurgical Extracardiac Vascular Shunts in the Thorax: Clinical and Imaging Characteristics,” Radiographies 30(5): e41; Freeman, J. A. and Woods, T. D. 2008. “Use of Saline Contrast Echo Timing to Distinguish Intracardiac and Extracardiac Shunts: Failure of the 3- to 5-beat Rule,” Echocardiographry 25(10): 1127-1130; Schwartz, M. L., GauvreauL, K., del Nido, P., Mayer, J. E., and Colan, S. D. 2004. “Long-term Predictors Aortic Root Dilation and Aortic Regurgitation after Arterial Switch Operation,” Online, Circulation (Hagerstown, Maryland) 110(11 Supplement 1): 11 128-132; Wang, Z. J., Reddy, G. P., Gotway, M. B., Yeh, B. M., and Higgins, C. B. 2003. “Cardiovascular Shunts: M R Imaging Evaluation,” Radiographies 23(1): S181-S194). In contrast, here will be described intentional extracardiac shunting to reverse transposition of the great arteries as a defect that originates within the heart.

While essentially the same as the alleviation of hypertension from a vessel by drawing off and diverting a portion of its flow to another larger vessel as addressed below in section 2b(2)(h)(h4), entitled The ‘Tuned’ Shunt in the Relief of Localized Vascular Hypertension, the exchange of flow between the aorta and pulmonary arteries doubles this relation and diverts the flow through each vessel in its entirety. All degrees of severity and forms of associated malformations are encountered with congenital heart defects to include complex transposition or discordance of the great arteries, so that, for example, some proportion or all of the blood normally passed through either vessel is delivered into the other.

Unless bidirectional valves to be described are used, ordinary ductus side-entry jackets could provide such crossover shunting, but solenoid-driven valves can be switched to facilitate accomplishment of the end sought. References pertaining to the diagnosis of transposition are provided above in section 2a(2), entitled Inventive Content-pertinent Fetal Diagnostics.

Where the positions of the great arteries, specifically the aorta and the pulmonary trunk, are fully reversed so that each passes the blood normally passed by the other, the flow through each can be crossed over or transferred into the other less traumatically than through a conventional arterial switch operation (see, for example, Szymanski, M. W., Moore, S. M., Kritzmire, S. M., and Goyal, A. 2023. “Transposition of the Great Arteries, Online, Treasure Island, Florida: StatPearls Publishing Co; Kumar, T. K. S. 2020. “Congenitally Corrected Transposition of the Great Arteries,” Online, Journal of Thoracic Disease (Hong Kong, China) 12(3): 1213-1218).

If involved, using side-entry vascular diversion jackets to bypass a stenotic segment, the more traumatic Rastelli procedure can be avoided thus eliminating all transection and interruption in blood flow (see, for example, Alsoufi, B., Awan, A., Al-Omrani, A., Al-Ahmadi, M., Canver, C. C., and 3 others 2009. “The Rastelli Procedure for Transposition of the Great Arteries: Resection of the Infundibular Septum Diminishes Recurrent Left Ventricular Outflow Tract Obstruction Risk,” Online, Annals of Thoracic Surgery (Amsterdam, North Holland, Netherlands) 88(1): 137-143).

As schematically depicted in FIG. 11, crossover, or translocation of flow is obtained with solenoid-driven vascular valves to simultaneously switch the diversion chutes 18 from fully retracted, of withdrawn, as shown in the left-hand view of FIG. 11 to fully extended as shown in the right-hand view, thus redirecting all flow through each into the other. To eliminate any medically significant interruption in blood flow or mixture of blood between the two vessels, the meticulously pretested valves are actuated from the same switch to function at exactly the same instant. This eliminates the need to transect both vessels, and is accomplished without interruption in the flow of blood through either. Furthermore, druglines in FIG. 11 marked part number 8 remain to allow the direct pipe-targeting to either or both crossovers of any medication or valve supportive agent as necessary.

Where some less than complete mixture of the flows is involved, the conventional arterial switch procedure must be used; however, even within the context of complete transposition as rare, incomplete transposition of flow is even rarer (see, for example, Breining, H. 1971. Partielle Transpositionen der grossen Gefasse [partial transposition of the great vessels],” (in German), Zeitschrift für Kreislaufforschung [Journal for Circulatory Research] 60(2): 151-161; Lepore, A. A. and Gomez, R. A. 1962. “Partial Transposition of Great Vessels with Overriding Pulmonary Artery: A Diagnostic Study,” Journal of Cardiovascular Surgery (Turin, Italy) 3:169-181).

Clarification of the terms partial and incomplete in relation to transposition is recommended (see, for example, Roderick, M. E., Maria, N., Crucean, A., Stickley, J., Barron, D. J., and Anderson, R. H. 2021. “Defining Transposition: What Have We Learnt?,” Online, Annals of Pediatric Cardiology (Mumbai [Bombay], India) 14(1): 63-66).

Today, due to advancements in imaging technology and medical diagnostics addressed above in section 2. Background of the Invention. a. General. (2). Inventive Content-pertinent Fetal Diagnostics, for the operator to enter and discover an unforeseen complication or anomaly of anatomy occurs rarely if ever. Where a repair predicted to be satisfactory encounters some insuperable obstruction, a “bail out” strategy is to perform the procedure for immediate conversion to a transplant, or if the repair itself will not do, then a means for the temporarily bridging to a transplant such as the placement of an artificial heart. When possible, endoscopic extracardiac repair with the aid of transcatheteric or transluminal techniques is less traumatizing and precarious than is intracardiac surgery in an open field.

To further minimize trauma and healing time, transposition-associated defects of the heart valves or septum, for example, are best repaired transcatheterically or transluminally or if resistant thereto, then endoscopically, and collateral complications of internal medicine identified and treated periprocedurally (see, for example, Al-Zahrani, R. S., Alharbi, S. H., Tuwaijri, R. M. A., Alzomaili, B. T., Althubaiti, A., and Yelbuz, T. M. 2018. “Transposition of the Great Arteries: A Laterality Defect in the Group of Heterotaxy Syndromes or an Outflow Tract Malformation?,” Annals of Pediatric Cardiology 11(3): 237-249; Horer, J. 2018. “Current Spectrum, Challenges and New Developments in the Surgical Care of Adults with Congenital Heart Disease,” Cardiovascular Diagnosis and Therapy 8(6): 754-764; Kiener, A., Kelleman, M., McCracken, C., Kochilas, L., St Louis, J. D., and Oster, M. E. 2018. “Long-term Survival after Arterial versus Atrial Switch in d-Transposition of the Great Arteries,” Annals of Thoracic Surgery 106(6): 1827-1833; Moe, T. G. and Bardo, D. M. E. 2018. “Long-term Outcomes of the Arterial Switch Operation for d-Transposition of the Great Arteries,” Progress in Cardiovascular Diseases 2018 61(3-4): 360-364; Anderson, R. H. and Stickley, J. 2018. “Is Transposition a Defect of Laterality?,” Annals of Pediatric Cardiology 11(3): 235-236; Bokenkamp, R., Aguilar, E., van der Palen, R. L., Sojak, V., Bruggemans, E. F., and 3 others 2016. “Reoperation for Right Ventricular Outflow Tract Obstruction after Arterial Switch Operation for Transposition of the Great Arteries and Aortic Arch Obstruction,” European Journal of Cardiothoracic Surgery 49(5): e91-e96; Baruteau, A. E., Vergnat, M., Kalfa, D., Delpey, J. G., Ly, M., and 3 others 2016. “Long-term Outcomes of the Arterial Switch Operation for Transposition of the Great Arteries and Ventricular Septal Defect and/or Aortic Arch Obstruction,” Interactive Cardiovascular and Thoracic Surgery 23(2): 240-246; Chang, Y., Li, S., Zhang, H., Hua, Z., Yang, K., and Gao, H. 2016. “The Functional Status of Neoaortic Valve and Left Ventricular Outlet Tract after Arterial Switch Operation for Transposition of Great Arteries with Left Ventricular Outlet Tract Obstruction,” Interactive Cardiovascular and Thoracic Surgery 23(1): 9-13; Matsuba, T., Shigehisa, Y., Imagama, 1., and Imoto, Y. 2016. “A Case of Arterial Switch Operation with Coronary Elongation Technique,” Surgical Case Reports 2(1): 7; Orphanet/U.S. Department of Health and Human Services, National Institutes of Health, National Center for Advancing Translational Sciences, Genetic and Rare Diseases Information Center Undated 2015 “Conotruncal Heart Malformations,” Online at https://rarediseases.info.nih.gov/diseases/8189/conotruncal-heart-malformations; Marathe, S. P. and Talwar, S. 2015. “Surgery for Transposition of Great Arteries: A Historical Perspective,” Annals of Pediatric Cardiology 8(2): 122-128 Hermsen, J. L. and Chen, J. M. 2015. “Surgical Considerations in d-Transposition of the Great Arteries,” Seminars in Cardiothoracic and Vascular Anesthesia 19(3): 223-232; Haeffele, C. and Lui, G. K. 2015. “Dextro-transposition of the Great Arteries: Long-term Sequelae of Atrial and Arterial Switch,” Cardiology Clinics 33(4): 543-558, viii; Mbuagbaw, L., Forlemu-Kamwa, D., Chu, A., Thabane, L., and Dillenberg, R. 2014. “Outcomes after Corrective Surgery for Congenital Dextro-transposition of the Great Arteries Using the Arterial Switch Technique: A Protocol for a Scoping Systematic Review,” Online, British Medical Journal Open 4(6): e005123; Mah, D. Y., Alexander, M. E., Banka, P., Abrams, D. J., Triedman, J. K., and 4 others 2013. “The Role of Cardiac Resynchronization Therapy for Arterial Switch Operations Ccomplicated by Complete Heart Block,” Annals of Thoracic Surgery 96(3): 904-909; Unolt, M., Putotto, C., Silvestri, L. M., Marino, D., Scarabotti, A., Massaccesi, V., Caiaro, A., Versacci, P., and Marino, B. 2013. “Transposition of Great Arteries: New Insights into the Pathogenesis,” Online, Frontiers in Pediatrics 1:11; Gabriel, J., Scheld, H. H., Tjan, T. D., Osada, N., and Krasemann, T. 2011. “Is the Function of All Cardiac Valves after the Arterial Switch Operation Influenced by an Associated Ventricular Septal Defect?,” Cardiology in the Young (Cambridge, England) 21(4): 383-391; Alsoufi, B., Awan, A., Al- Omrani, A., Al-Ahmadi, M., Canver, C. C., and 3 others 2009. “The Rastelli Procedure for Transposition of the Great Arteries: Resection of the Infundibular Septum Diminishes Recurrent Left Ventricular Outflow Tract Obstruction Risk,” Annals of Thoracic Surgery 88(1): 137-143; Martins, P. and Castela, E. 2008. “Transposition of the Great Arteries,” Orphanet Journal of Rare Diseases 3:27; Watanabe, M., Choudhry, A., Berlan, M., Singal, A., Siwik, E., Mohr, S., and Fisher, S. A. 1998. “Developmental Remodeling and Shortening of the Cardiac Outflow Tract Involves Myocyte Programmed Cell Death,” Development (Cambridge, England) 125(19): 3809-3820; Pasquini, L., Sanders, S. P., Parness, I. A., Colan, S. D., Van Praagh, S., Mayer, J. E. Jr., and Van Praagh, R. 1993. “Conal Anatomy in 119 Patients with D-loop Transposition of the Great Arteries and Ventricular Septal Defect: An Echocardiographic and Pathologic Study,” Journal of the American College of Cardiology 21(7): 1712-1721; Goor, D. A. and Edwards, J. E. 1973. “The Spectrum of Transposition of the Great Arteries: With Specific Reference to Developmental Anatomy of the Conus,” Circulation 48(2): 406-415; Goor, D. A., Dische, R., and Lillehei, C. W. 1972. “The Conotruncus. I. Its Normal Inversion and Conus Absorption,” Circulation 46(2): 375- 384; Mitchell, S. C., Korones, S. B., and Berendes, H. W. 1971. “Congenital Heart Disease in 56,109 Births. Incidence and Natural History,” Circulation 43(3): 323-332).

Whether this can be remedied without the need for open heart surgery depends upon the degree of admixture, or the relative proportion of oxygenated blood passed into the pulmonary artery and unoxygenated blood into the aorta. In some cases of less than completely exchanged, or admixed blood, the combination of the extracardiac procedure described here and a Rashkind transcatheteric or transluminal septostomy or septectomy (van der Horst, R. L., Winship, W. S., and Gotsman, M. S. 1970. “Balloon Atrial Septostomy in Complete Transposition of the Great Vessels,” South African Medical Journal 44(32): 916-919; Rashkind, W. J. and Miller, W. W. 1966. “Creation of an Atrial Septal Defect without Thoracotomy. A Palliative Approach to Complete Transposition of the Great Arteries,” Journal of the American Medical Association 196(11): 991-992) may achieve oxygenation sufficient to avoid developmental impairment secondary to chronic hypoxia without open heart surgery.

In this way, the need for an open heart repair might remain unnecessary to the end of life. In an extracardiac repair of a complete or dextro-transposition of the great arteries using valve-jackets, neither the great nor the coronary arteries are transected and switched; instead, all vessels requiring redirection are corrected using the same double reversal shunting, or cross-flow means Whereas tapping off a fraction of the blood moving through the pulmonary or portal vein is unidirectional for pressure relief, vessel switching to exchange unoxygenated for oxygenated blood and the reverse in an extracardiac complete or dextro-transposition is bidirectional.

FIGS. 11 thru 13B show that manually operated unidirectional vascular valves can be used to perform an extracardiac correction of complete or dextro-transposition of the great arteries. The two way arrows indicate that to protect the segments of the vessels between the superior and inferior pair of valves, the implanted control microprocessor is programmed to periodically retract the diversion chutes, allowing blood to flow uninterrupted through the transposed vessels. Where this occasional and short-term reversion to the defective state is ill-advised, bidirectional valves, shown in FIGS. 2-4 eliminate the intervening segments superior and inferior to the valve-jackets. Retractable valves must be remotely controlled and therefore cannot be onetime manually controlled in a prosthesis for diverting urine from a ureter, for example.

Conventional arterial switch is almost always successful; however, its open heart trauma limits it to neonates—Rashkind balloon septostomy and/or prostaglandin E1 (alprostadil) if appropriate notwithstanding—are not able to withstand the procedure or who would achieve a better outcome if allowed to wait until they attain sufficient robustness. Prostaglandin carries potentially fatal side effects, making the combination of extracardiac correction and septostomy preferred, especially if the combination continues to allow sufficient oxygenation over the long-term.

Depending upon its severity, chronic hypoxia may permit survival but not normal development. Exigent measures such as creating or enlarging a ventricular septal defect and/or administering prostaglandin to keep the ductus arteriosus patent so that oxygenated blood will mix with that unoxygenated is an expedient that can sustain a neonate but is not sufficient for the neonate to thrive. The extracardiac correction of complete or dextro-transposition through the use of valves is limited to cases in which the degree of discordance results in a mixing of unoxygenated with oxygenated blood which is not sufficient to induce a state of chronic hypoxia.

When this is not the case, the procedure can be used as a bridging strategy, either alone, where depending upon the degree of mixing, it can be significantly more effective than a balloon septostomy or septectomy and/or prostaglandin, or in combination with these. When due to the degree of discordance, oxygenated blood is not adequately isolated from that unoxygenated, two courses of action are presented. If the mixing can be completely or substantially eliminated, open heart surgery is obligatory. If not, the condition requires that the defective heart be replaced. Non open heart and great and coronary vessel non-transecting repair offered by synthetic components is considerably less traumatic and averts the need for less effective measures that sustain mixing of oxygenated and unoxygenated blood to defer treatment during which hypoxia impairs the normal development of all organs and tissues.

The components devised to allow much growth, even in the very young, the procedures delineated below should last for years. Precisely controllable, as shown in FIGS. 11 and 12, vascular servovalves can be applied to extracardiac proportional correction of partial or complete dextro-transposition or discordance, materially reducing the trauma of repair in a neonate compared to a Jatene arterial, much less the earlier atrial switch operations, with or without a Rashkind balloon atrial septostomy if appropriate (see, for example, Villafane, J., Lantin-Hermoso, M. R., Bhatt, A. B., Tweddell, J. S., Geva, T., Nathan, M., Elliott, M. J., and 7 others 2014. “D-transposition of the Great Arteries: The Current Era of the Arterial Switch Operation,” Journal of the American College of Cardiology 64(5): 498-511; Martins, P. and Castela, E. 2008. “Transposition of the Great Arteries,” Orphanet Journal of Rare Diseases 3:27).

Dynamic blood pressure responsive control over chute displacement is exercised by one or more strain-gauge based sensor output-driven microcontrollers. Constantly changing in proportion to the mean bodily blood pressure, pulmonary arterial and portal vein hypertension and partial or complete dextro-transposition or discordance of the great arteries call for dynamic response.

The procedure is not open heart, and there is no transection of the great vessels, avoiding the risks such as postoperative problem bleeding or stenosis at the anastomoses associated therewith, duration of the procedure is much shorter, little if any transfusion and the adverse reactions this might induce such as problem bleeding, clotting, stroke, cardiac arrest, cognitive impairment, less risk of inducing dyspnea, dysrhythmia, or injury to the recurrent laryngeal nerve with consequent speech impairment.

The means to be described seek to exchange or cross-divert blood between the great arteries so that these will flow properly through the pulmonary and systemic circulations. The object is to allow the avoidance of open heart surgery to correct ventriculoarterial discordance. Because the procedure to place the diverting jackets involves much less trauma than conventional surgery, a secondary object is to make possible application of the procedure in prenates and neonates. Left ventricular outflow tract obstruction is repaired before, and a ventricular septal defect repaired transluminally following the switch, with a patent ductus arteriosus repaired following the switch by conventional extracardiac surgery.

If neither a sufficient septal defect or patent ductus arteriosus is present, immediate ventriculoarterial switching averts the need for prostaglandin E1 to sustain ductus patency or balloon atrial septostomy to initiate sufficient mixing of oxygenated with unoxygenated blood. In 1971 when Fontan and Baudet described their procedure (Fontan, F. and Baudet, E. 1971. “Surgical Repair of Tricuspid Atresia,” Thorax 26(3): 240-248), heart transplantation had not achieved a level of success that would allow it as a reasonable alternative to surgical repair. Time has reversed this relation—today, heart transplantation, especially in the very young, offers a critically superior outcome. Moreover, within 15 years, the 15 or so years until a second heart transplant is likely to be needed should be overcome.

Where maldevelopment due to circulatory insufficiency and/or early death is predicted to ensue, the patient is too frail to undergo more radical surgery, or the condition does not lend itself to a good outcome following a conventional repair, extracardiac ventriculoarterial switching, if necessary, with a Rashkind balloon interatrial septostomy, prostaglandin E1, can serve as a bridging strategy pending heart transplantation as the option of choice. Incision is preferably limited to the gaining endoscopic access, however, if necessary, an open surgical field can be used with neither the great nor the coronary arteries transected.

Eliminating all incisions not essential to expose the heart, the cross-flow piping procedure is critically less traumatic, can be applied immediately upon diagnosis, and significantly reduces the risks of peri- and postoperative complications such as bleeding and the need for a chest tube, as well as stenosis at transection sites along the great and coronary arteries. Furthermore, the use of synthetic components facilitates the incorporation of permanent miniature heart monitoring and telemetering devices, such as a blood pressure monitor, and furnishes a secure mounting for associated devices, such as a cardioverter-defibrillator. Prenatal echocardiography is beneficial in alerting not just the presence but the conformation of defects so that treatment can be ascertained prior to birth.

An atrial septostomy may be used to sustain a neonate or infant with an unsurvivably malformed heart or an adult with an irreparably impaired heart as a preparatory step to the fitting of an artificial heart as a bridging strategy to a heart transplant (Kozik, D. and Alsoufi, B. 2023. “Pediatric Mechanical Circulatory Support—A Review,” Indian Journal of Thoracic and Cardiovascular Surgery,” (New Delhi, India) 39 (Supplement 1): 80-90; Lynch, A., Jeewa, A, Maurich, A., Mazwi, M., Jean-St-Michel, E., and 5 others 2023. “Canada's First Successful Paediatric Total Artificial Heart Implant,” Online, Canadian Journal of Cardiology Pediatric and Congenital Heart Disease 2(6 Part B): 494-497; Villa, C. R., Moore, R. A., Morales, D. L., and Lorts, A. 2020. “The Total Artificial Heart in Pediatrics: Outcomes in an Evolving Field,” Online, Annals of Cardiothoracic Surgery (Hong Kong, China) 9(2): 104-109; Koppert, E., Holfert, J. W., Dew, P. A., Juretich, J. T., Diegel, P. D., and Burns, G. L. 1991. “Preliminary in Vitro Evaluation of the First Neonatal Total Artificial Heart,” American Society for Artificial Internal Organs Transactions 37(3): M127-128; Koppert, E., Holfert, J. W., Dew, P. A., Pauley, J., Pantalos, G. M., Crump, K. R., and Burns, G. L. 1990. “A Total Artificial Heart for Neonates Allowing Bridging to Transplantation,” American Society for Artificial Internal Organs Transactions 36(3): M226-M230)(see also, for example, Cedars. A., Vanderpluym, C., Koehl. D., Cantor, R., Kutty, S., and Kirklin, J. K. 2018. “An Interagency Registry for Mechanically Assisted Circulatory Support (INTERMACS) Analysis of Hospitalization, Functional Status, and Mortality after Mechanical Circulatory Support in Adults with Congenital Heart Disease,” Journal of Heart and Lung Transplantation (New York, New York) 37(5): 619-630; VanderPluym, C. J., Cedars. A., Eghtesady, P., Maxwell, B. G., Gelow. J. M., and 5 others 2018. “Outcomes following Implantation of Mechanical Circulatory Support in Adults with Congenital Heart Disease: An Analysis of the Interagency Registry for Mechanically Assisted Circulatory Support (INTERMACS),” Journal of Heart and Lung Transplantation (New York, New York) 37(1): 89-99; Glynn, J., Song. H., Hull, B., Withers, S., Gelow. J., Mudd, J., Starr, A., and Wampler, R. 2017. “The OregonHeart Total Artificial Heart: Design and Performance on a Mock Circulatory Loop,” Artificial Organs 41(10): 904-910; Cook, J. A., Shah, K. B., Quader, M. A., Cooke, R. H., Kasirajan, V., and 4 others 2015. “The Total Artificial Heart,” Journal of Thoracic Disease 7(12): 2172-2180. Op.cit.; Copeland, J. G., Arabia, F. A., Tsau, P. H., Nolan, P. E., Mcclellan, D., Smith, R. G., and Slepian, M. J. 2003. “Total Artificial Hearts: Bridge to Transplantation,” Cardiology Clinics 21(1): 101-113; Koppert, E., Holfert, J. W., Dew. P. A., Juretich, J. T., Diegel, P. D., and Burns, G. L. 1991. “Preliminary in Vitro Evaluation of the First Neonatal Total Artificial Heart” American Society for Artificial Internal Organs Transactions 37(3): M127-M128.

Whether due to the degree of malformity, trauma, or the acumen of the surgery relative thereto, a well designed artificial heart can achieve pulmonary and systemic circulation superior to that attainable through an inadequate repair, and in a neonate or infant, it is critical that early development not be impaired by a condition of chronic hypoxia. That bypass is through a mechanical device facilitates the incorporation of various diagnostic and therapeutic devices and allows the directly pipeline targeted use of drug delivery channels from a small port at the body surface.

The automatically dispensed direct targeting of an antithrombotic drug through the artificial heart is a significant benefit (see, for example, Volod, O., Lam, L. D., Lin, G., Kam, C., Kolyouthapong, K., Mac. J., Mirocha, J., and 3 others 2017. “Role of Thromboelastography Platelet Mapping and International Normalized Ratio in Defining “Normocoagulability” During Anticoagulation for Mechanical Circulatory Support Devices: A Pilot Retrospective Study,” American Society for Artificial Internal Organs Journal 63(1): 24-31; Ramirez, A., Riley, J. B., and Joyce, L. D. 2016. “Multi-targeted Antithrombotic Therapy for Total Artificial Heart Device Patients,” Journal of Extracorporeal Technology 48(1): 27-34; Jennings. D. L., Horn, E. T., Lyster, H., Panos, A. L., Teuteberg. J. J., Lehmkuhl. H. B., Perez, A., and Shullo. M. A. 2016. “Assessing Anticoagulation Practice Patterns in Patients on Durable Mechanical Circulatory Support Devices: An International Survey,” American Society for Artificial Internal Organs Journal 62(1): 28-32).

One example of a congenital condition requiring a heart transplant is a complete or dextro-transposition of the great arteries wherein, rather than simply reversing the function of each so that either oxygenated or unoxygenated blood substantially if not exclusively is carried in the opposite vessel making adequate repair through reversal possible, both the aorta and main pulmonary artery feed a mixture of oxygenated and unoxygenated blood refractory to satisfactory repair (see, for example, Goor, D. A. and Edwards, J. E. 1973. “The Spectrum of Transposition of the Great Arteries: With Specific Reference to Developmental Anatomy of the Conus,” Circulation 48(2): 406-415).

Where latitude in the rotational orientation of the transplant offered by jacket-to-jacket flexible connection outweighs the risk of a dysrhythmia, an anticoagulant such as warfarin oral, or a clot-bound factor Xa inhibitor, preferably apixaban or rivaroxaban with the reversal agent andexanet alfa available should it be approved, or a drip interval regulated by a timing control module or microprocessor that controls a small flat drug reservoir subdermally positioned in the pectoral region, and/or a pacemaker is planed from the outset. Such malformities include marked ectopias in the form of cyanotic or dextro-(complete), and noncyanotic or levo-(congenitally corrected) transpositions of the great vessels.

When the relative proportion of blood to be diverted from the one vessel to the other can be ascertained before entry, conventional side-entry diversion jackets with side connectors angled to minimize shear stress on the intimae at either end and of the proper internal diameter are used. When the optimal volume and flow rate of diversion cannot be foretold but require to be gradually adjusted with the diversion in position, a ductus side-entry jacket diversion servovalve is used as a trimmer valve to establish optimal flow.

The Taussig-Bing anomaly shares features with dextro-transposition but repair is largely intracardiac; hence, to what if any extent the current technology might participate in repair depends upon the specific condition (see, for example, Mavroudis, C. and Backer, C. L. (eds.) 2015. Atlas of Pediatric Cardiac Surgery, New York, New York: Springer; Mavroudis, C. and Backer, C. L. (eds.) 2013. Pediatric Cardiac Surgery, New York, New York: Wiley-Blackwell; Litwin, S. B 2007. Color Atlas of Congenital Heart Surgery, New York, New York: Springer; Devaney, E. J., Charpie, J. R., Ohye. R. G., and Bove. E. L. 2003. “Combined Arterial Switch and Senning Operation for Congenitally Corrected Transposition of the Great Arteries: Patient Selection and Intermediate and Intermediate Results,” Journal of Thoracic and Cardiovascular Surgery 125(3): 500-507).

Broadly, when intracardiac repair is needed, the means described herein and in U.S. Pat. No. 11,759,186, FIGS. 16 thru 22 can still be applied to the extracardiac components. With any type repair, the means described herein can be used to directly target drugs to the treatment site, organ, or supply territory. Previously, dextro-transposition could be no more than palliated with adverse sequelae through various measures such as enlarging septal defects to increase the mixture of oxygenated, or saturated, and deoxygenated, or unsaturated (oxygen-depleted) blood.

Later procedures to correct a complete or dextro-transposition, to include the Jatene arterial switch, produced better outcomes, but still required incision into the heart to cut free and mobilize the vessels so that these could be shifted in relative position, the detractions of open-heart surgery then not avoidable (see, for example, Da Silva, J. P., Da Silva, L. F., Baumgratz, J. F., Castro, R. M., Bezerra, R. F., and Guilhen, J. C. 2015. “Root Translocation in Congenitally Corrected Transposition of the Great Arteries with Ventricular Septal Defect and Pulmonary Stenosis, and Other Lesions,” Seminars in Thoracic and Cardiovascular Surgery. Pediatric Cardiac Surgery Anual 2015 18(1): 34-39; Konstantinov, I. E., Alexi-Meskishvili, V. V., Williams, W. G., Freedom, R. M., and Van Praagh, R. 2004, Op Cit. above in section 2b(1)(b)(b3), entitled Neonatal and Infant Compound vascular bypass, or sudden switched-organ Transplantation of the Heart). As with the Jatene procedure, to preclude the development of heart failure, infection, and/or pulmonary hypertension which obtain with protracted hypoxia due to desaturated blood in the systemic circulation and oxygenated or saturated blood in the pulmonary circulation, the procedure must be performed within the first month of life.

When simple, or relatively isolated, and unaccompanied by multiple serious structural defects, levo-transposition may not require reconstructive surgery. When complex, or complicated by the compresence of one or more septal defects such as an intracardiac shunt, patent ductus arteriosus, stenotic valve, or strictured vessel, surgical correction is essential, the Senning-Rastelli procedure to attain atrioventricular and ventriculoarterial concordance referred to as a “double switch” (see, for example, Lacour-Gayet. F., Bove, E. L., Hraska, V., Morell, V. O., Spray, T. L. (eds.) 2016. Surgery of Conotruncal Anomalies, New York, New York: Springer; Ilyinas. A. S., Teplov. P. V., Sakovich, V. A., and Ohye. R. G. 2016. “Surgical Technique of Double Switch Procedure: Senning with Arterial Switch Operation for Congenitally Corrected Transposition of the Great Arteries with Ventricular Septal Defect,” Multimedia Manual of Cardiothoracic Surgery 2016 pii: mmw007; Mavroudis, C. and Backer, C. L. (eds.) 2015, Op cit.; Cui, B., Li, S., Yan, J., Shen, X., Wang, X., and 4 others 2014. “The Results of a Two-stage Double Switch Operation for Congenital Corrected Transposition of the Great Arteries with a Deconditioned Morphologically Left Ventricle,” Interactive Cardiovascular and Thoracic Surgery 19(6): 921-925; Mavroudis, C. and Backer, C. L. (eds.) 2013. Op cit., Ly, M., Belli, E., Leobon, B., Kortas, C., Grollmuss, O. E., Piot, D., Planche, C., and Serraf. A. 2009. “Results of the Double Switch Operation for Congenitally Corrected Transposition of the Great Arteries,” European Journal of Cardiothoracic Surgery 35(5): 879-884; Litwin, S. B 2007, Ishibashi, N., Aoki, M., and Fujiwara. T. 2004. “Double Switch Operation in a Young Infant,” Cardiology in the Young (Cambridge, England) 14(6): 661-663; Devaney, E. J., Charpie, J. R., Ohye, R. G., and Bove, E. L. 2003, Op cit; Reddy, V. M., McElhinney, D. B., Silverman, N. H., and Hanley, F. L. 1997. “The Double Switch Procedure for Anatomical Repair of Congenitally Corrected Transposition of the Great Arteries in Infants and Children,” European Heart Journal 18(9): 1470-1477).

The object sought in the application of vascular valves and servovalves to the correction of transpositions of the great arteries where intracardiac anomalies are absent is to avoid the need to enter the heart; both switching flow between the aorta and main pulmonary artery and the creation of a neoaortic arch whereby the coronary arteries are moved from the aorta to the main pulmonary artery—in effect, creating a surrogate aorta—are accomplished without the need to enter the heart, which necessitating cardiopulmonary support and general anesthesia, risks their many serious adverse sequelae.

If one or more coronaries will not reach the pulmonary artery, then using the means described, this is accomplished in either of two ways. To anastomose the coronary to the pulmonary artery, the coronary is transected along its length and a section interposed by attaching a ductus side-entry jacket toward the end of either stump and connecting the jacket mainlines with a length of a suitable bisphenol A and residual plasticizer-free polymeric tubing.

To avert the risk of migration were the free ends of the stumps extending out of the jackets to contract, these are filled with a nonallergenic and nontoxic monomer or residual monomer and antibiotic-releasing bone cement or an acrylate-based dental cement and sutured shut. Further stabilization of the jackets is by ‘-pexying’ to suitable neighboring tissue by passing suture through the jacket suture loops or eyelets, part number 15 in the drawing figures. The jackets are inherently positioned to deliver drug support to these connections from a small port at the body surface, the method for replenishing and automating the release of medication addressed below.

The anastomoses joining the coronary and main pulmonary artery can be drug targeted through the accessory channel of a jacket on the pulmonary artery placed upstream (inferior to) the anastomoses. The other method is to place a jacket on the pulmonary artery having as many side connectors as coronaries to be connected and running the mainlines to connect with the free proximal cut ends of the stumps to which the mainlines are connected by a tiny jacket on each, with the free ends extending out of the jackets shut off as indicated and shown in U.S. Pat. No. 11,759,186, FIGS. 16 thru 22.

In addition to avoiding the need to enter the heart, the use of side-entry diversion jackets as vascular valves to enable extracardiac cross-circulation between ectopic, or reversed, great arteries makes possible the direct targeting to the site of the procedure of drugs to forestall if not eliminate adverse sequelae such as pulmonary stenosis, without or with graft patching or stenting (see, for example, Ghorbnazadeh, A., Zirak, N., Fazlinezhad, A., Moenipour, A., Manshadi, H. H., and Teshnizi, M. A. 2017. “Situs Inversus with Levocardia and Congenitally Corrected Transposition of Great Vessels in a 35 Year Old Male: A Case report,” Electronic Physician 9(1): 3570-3574; El-Zein, C., Subramanian, S., and Ilbawi, M. 2015. “Evolution of the Surgical Approach to Congenitally Corrected Transposition of the Great Arteries,” Seminars in Thoracic and Cardiovascular Surgery. Pediatric Cardiac Surgery Anual 18(1): 25-33; Da Silva, J. P., Da Silva, L. F., Baumgratz, J. F., Castro, R. M., Bezerra, R. F., and Guilhen, J. C. 2015, Op cit, Lee, J. H., Park, E. A., Lee, W., Cho, H. J., Kim, K. B., and Hwang, H. Y. 2014 Op cit.; Delmo Walter, E. M. and Hetzer, R. 2013. “Surgical Treatment Concepts for End-stage Congenital Heart Diseases,” HSR (Health Services Research) Proceedings in Intensive Care and Cardiovascular Anesthesia 5(2): 81-84; Martins, P. and Castela, E. 2008. “Transposition of the Great Arteries,” Orphanet Journal of Rare Diseases 3:27; Sue, S. H., Wei, J., Chuang, Y. C., Chang, C. Y., Lee, W. C., and Lee, S. L. 2008, Op cit.; Marelli, D., Silvestry, S., Zwas, D., Mather, P., Rubin, S., and 5 others 2007. “Modified Inferior Vena Caval Anastomosis to Reduce Tricuspid Valve Regurgitation after Heart Transplantation,” Texas Heart Institute Journal 34(1): 30-35; Devaney, E. J., Charpie, J. R., Ohye, R. G., and Bove, E. L. 2003. “Combined Arterial Switch and Senning Operation for Congenitally Corrected Transposition of the Great Arteries: Patient Selection and Intermediate Results,” Journal of Thoracic and Cardiovascular Surgery 125(3): 500-507; Rabago, G., Martin-Trenor, A., Lopez-Coronado, J. L., Macias, A., Cosin-Sales, J., and Herreros, J. M. 2002. “Bicaval Anastomosis in a Heart Transplant Recipient with Left Superior Vena Cava,” Annals of Thoracic Surgery 74(4): 1242-1244; Carrel, T. and Pfammatter, J. P. 2000. “Complete Transposition of the Great Arteries: Surgical Concepts for Patients with Systemic Right Ventricular Failure Following Intraatrial Repair,” Thoracic and Cardiovascular Surgeon 48(4): 224-227; Bove, E. L. 1995. “Congenitally Corrected Transposition of the Great Arteries: Ventricle to Pulmonary Artery Connection Strategies,” Seminars in Thoracic and Cardiovascular Surgery 7(3): 139-144; Blanche, C., Valenza, M., Czer, L. S., and Trento, A. 1994, Op cit.; Jebara, V. A., Dreyfus, G., Acar, C., Deloche, A., Couetil, J. P., Fabiani, J. N., and Carpentier, A. 1990 Op cit.; Chartrand, C., Guerin, R., Kangah, M., and Stanley, P. 1990. “Pediatric Heart Transplantation: Surgical Considerations for Congenital Heart Diseases,” Journal of Heart Transplantation 9(6): 608-617).

2b(2)(b). Bidirectional Valves for Perinatal Extracardiac Correction of Complete or Dextro-Transposition of the Great Arteries and Reciprocal Cross-Circulation

The small size of the perinate and presentation of space limitations in patients at any age recommend the incorporation of features that allow the minimization in dimensions and weight of vascular valves and servovalves. Shown in FIGS. 11 thru 13B, to allow for growth, side-entry diversion valves should be no smaller than necessary with inline connectors fully expandable. Of the applications for vascular valves and servovalves addressed here, only the need to establish exchanged flow or reciprocal cross-circulation between vessels in a single patient, as in an extracardiac, that is, open chest/closed heart, correction of a transposition of the great arteries, requires placing inlet and outlet shunt valve-jackets on each vessel.

Extracardiac repair of a transposition of the great arteries assumes that the condition is not associated with a double outlet right or left ventricle which would necessitate intracardiac repair in any event, and if postoperatively still impaired, would recommend replacement with a fully competent heart. Unless both recipient and donor are infants or small research animals so that miniature valves are preferred, reciprocal cross-circulation between two subjects is with nonbidirectional valves, one each on each vessel. Where arteries and veins run alongside one another, bidirectional valves facilitate switch transplantation with valves about half the size, an important advantage in small patients such as neonates, infants, and smaller animals.

While small, the valves incorporate coils, armatures, and neodymium magnets which result in a weight likely to cause discomfort in ante- and postpartum application both medical and veterinary, mostly in conducting research (see, for example, Yasui, H., Nakazawa, M., Morishima, M., Miyagawa-Tomita, S., and Momma, K. 1995. “Morphological Observations on the Pathogenetic Process of Transposition of the Great Arteries Induced by Retinoic Acid in Mice,” Circulation 91(9): 2478-2486; Straw, R. C., Aronson, E. F., and McCaw, D. L. 1985. “Transposition of the Great Arteries in a Cat,” Journal of the American Veterinary Medical Association 187(6): 634-636; Koo, S. T., LeBlanc, N. L., Scollan, K. F., and Sisson D. D. 2016. “Complete Transposition of the Great Arteries with Double Outlet Right Ventricle in a Dog,” Journal of Veterinary Cardiology 18(2): 179-186).

The reduction in size and number of valves make bidirectional vascular valves and servovalves suited to the tiny vessels of perinates. The valves shown in FIGS. 1 and 2 can be used to divert flow from the source to the destination vessel in the same (concurrent), or the opposite (countercurrent), direction this pertinent to reciprocal cross-circulation between an organ donor and recipient. For example, in a unilateral orthotopic transplantation of a like-sided kidney, positioning donor and recipient in inverted relation allows the use of somewhat shorter mainlines. Where to do so creates no difficulty for support personnel, this is recommended.

Lined with viscoelastic polyurethane foam to accommodate the pulse and growth, and provided with accessory channels to deliver medication to the endothelium superior and inferior to the diversion chutes, access of the bloodstream to the small segments between the diversion chutes in bidirectional valves, while the need therefor is unlikely, also necessitates the occasional retraction of the diversion chutes, indicated by the two-way arrows.

In FIG. 13B, two pairs of bidirectional valves allow elimination of the need to revert to the defective state in order to allow blood to flow over the intervening segment. Here the double arrows indicate not an occasional reversion to the defective state but rather the switching from the one to the other pair of bidirectional valves. To be occasionally retracted, these valves cannot be one-time manually switched plunger solenoid-driven but are reversible solenoid, closed loop stepper, or servomotor-driven.

Where space is lacking and/or the valve-jackets should be as light in weight as possible, as in extracardiac repair of transposition of the great arteries in a prenate, neonate, or infant, two-way, or bidirectional valves, FIGS. 11 thru 13B, allow the number of valve-jackets to be halved. For frail patients, correction without open heart surgery can be critical. Bidirectional valves also eliminate the skipped segment, avoiding trauma, anesthesia time, and potential injury to the heart or conduction system are reduced to a degree that allows correction to be achieved and maldevelopment avoided where conventional intracardiac repair should not be risked. Provided the condition can be corrected by placing valve-jackets on vessels outside the heart, correction of conditions such as arteriovenous, ventriculoarterial, and/or arteriovenous discordance is possible. Correction in this context means the initiation of normal circulation to the degree that normal development will ensue.

Directional reversal with a bidirectional valve as shown in FIGS. 12 thru 13B is applicable to the extracardiac correction of conotruncal discordances. While procedural and anesthesia time are greater than when correction can be accomplished with valving alone, in some situations, valving can be combined with intracardiac surgery to reduce overall trauma. Where the discordance is not limited to the aorta and pulmonary artery, additional valve-jackets are placed to properly align the vessels to their proper supply fields. Valve-jackets provided with an adequate thickness of foam lining and elastic mainlines, an initial fitting should not only accommodate the pulse, but last no less than the first three or so years, after which growth will necessitate replacement with larger valve-jackets and mainlines or conventional repair.

Bidirectional valves can be manually, solenoid, closed loop stepper, or servomotor-driven. Degradation of the segments between the jackets is prevented either by periodically retracting or opening the diversion chutes to allow blood to pass through these segments, as indicated by the double arrows in FIGS. 13A and 13B, or, where the duration of the open interval is insufficient, a second pair of crossover valves and lines is positioned near to the first pair, the program then alternating open or nondiverting and closed or diverting time between the two, as shown in FIG. 13B. Use of the accessory channels to release medication into the space between the chutes is not adequate to sustain the intervening segment.

Substances delivered to the intima by the passing blood can be replicated; however, the pulsatile variation in forces that maintains tonus and stimulates intimal sustainment by blasts, pericytes (perivascular cells, Rouget cells) and pericyte-like cells will be missing. Function thus requires the use of reversible solenoid or servomotor driven valves. The automatic retraction of the diversion chutes is programmed into the local microcontroller of control microprocessor. Such conditions misdirecting flow without differential variations in blood pressure for a given heart rate, reversible damped nonsparking solenoid or servomotor-switched valve-jackets apply.

Where repair of septal defects, stenotic or leaking valves, complete atrioventricular canal defect, severe Ebstein's anomaly, patent foramen ovale, truncus arteriosus, and so on, appear in combination and/or severity as to defy transluminal repair, the correction of extracardiac defects with the aid of valve-jackets lessens dissection and procedural time. For example, in repairing total anomalous pulmonary venous connection, the atrial septal defect is closed using the transcatheter technique, while oxygenated blood from the pulmonary veins is redirected to the aorta by means of servovalve-switched vascular valve-jackets, if possible, this combination of techniques averting the need for open heart surgery.

Severe single ventricle defects and the tetralogy of Fallot, where multiple procedures would not initiate circulation sufficiently normal to avert secondary degradation in the other organs to include the brain are best responded to with sudden or metered switch heart transplantation. Bidirectional valves are not applicable to sudden or metered compound vascular bypass, or sudden switched-organ transplantation, where each valve-jacket is affixed to a single vessel and outflow is to the corresponding single valve-jacketed single vessel in a different body with flow in the same arterial or venous direction.

FIG. 11 shows the use of manually operated unidirectional vascular valves in an extracardiac correction of transposition of the great arteries, and FIGS. 12 and 13A show the same application of bidirectional vascular valves where a considerable reduction in valve and line bulk and dimensions accommodates the tiny vessels of perinates, that is, prenates, neonates, and young infants. A bidirectional intravascular inlet servovalve can outflow into two unidirectional outlet, or destination valves on different vessels to dynamically adjust the blood pressure among the three vessels Placement of a small manual bidirectional valve to create an arteriovenous fistula for hemodialysis or apheresis vascular access connects the vessels with reversal in the direction of flow with less dissection and less risk of infection than does an anastomosis.

Moreover, should complications arise, a tiny port on the inside of the wrist or inner surface of the thigh, for example, allows direct access to the valve and line through the accessory channel to directly target an antimicrobial and anti-inflammatory to the treatment site. In a two-way diversion chute, the lower surface of each chute is usually closed off, so that the one or two accessory channels exit on the upper surface of the upper and lower, or inverted chute.

### Definition of Shunts Passive and ‘Tuned,’ or Metered, and Bypasses Passive and Metered

1. Shunts move flow from one ductus into another where:
a. Passive, or ‘dumb,’ shunts move flow through at a volume set by the internal cross-sectional area of their inlet, and if used, exit, or outlet, connecting side-entry jackets which structural, is fixed, so that these cannot be adjusted;
b. Tuned, ‘smart,’ or metered shunts are entered through a vascular servovalve and possibly, an exit, or outlet servovalve where these can continuously vary the rate of in- and/or outflow so that depending upon the elasticity of the conduit joining the two, these can pass through flow detained at an elevated or lowered pressure, in response to the commands fed back to the controller in an implanted automatic disorder response system as described in U.S. Pat. No. 11,759,186 and copending application Ser. No. 17/689,880 based upon the pathophysiological sensor inputs;

2. Bypasses move flow around a defect or healing segment along a ductus to continue flow past that segment and back into the same ductus, where:
a. Passive, or ‘dumb,’ bypasses move flow through at a volume set by the internal cross-sectional area of their inlet, and if used, exit, or outlet, connecting side-entry jackets which structural, is fixed, so that these cannot be adjusted;
b. Tuned, ‘smart,’ or metered bypasses are entered through a vascular servovalve and possibly, an exit, or outlet servovalve where these can continuously vary the rate of in- and/or outflow so that depending upon the elasticity of the conduit joining the two, these can pass through flow detained at an elevated or lowered pressure, in response to the commands fed back to an implanted automatic disorder response system controller based upon the pathophysiological sensor inputs;

A bypass entered and exited through vascular servovalves can switch flow between the substrate native ductus and the bypass. This can be of considerable benefit when avoiding the native lumen while diseased, being diagnosed, or under treatment would expedite healing. An episodic or intermittent condition justifies leaving the bypass in place. When incorporated into a prosthetic disorder response system, sensors for biomarkers or analytes indicative of disease or malfunction can signal the implanted microcontroller or master control microprocessor to effect such switching between pathways automatically without the ambulatory patient even conscious of the action.

In this way, a malfunction that would result in serious injury if not death is averted instantly. In accordance with its prescription-program, an implanted automatic disorder response system controller can target medication to the site, and if implanted, electrical stimulation, for example, executed at the same time that it signals the clinic of the emergency and specifies its nature. Such function, especially important for patients such as the very young, the very old, and the mentally disturbed who lack the mental presence to take proper action, contact the clinic, and are prescription nonadherent can be critically supported.

Not only is the affected endothelium (or urothelium, or intestinal epithelium) given relief, but the treatment itself can consist of or include the release of medication through one or more bypass druglines, and if suitably equipped, electrostimulation, as commanded by the implanted system controller in accordance with its prescription-program in response to feedback from sensors stationed within the native ductus. The same pertains to the bypasses shown in FIGS. 22B thru 22E where the bypass druglines are numbered 8 and 8′.

Switching between the native lumen and the bypass does not leave the bypass filled with blood, which continues to flow through the one of the other. An extended period of no blood flow through the endothelium bypassed could leave it having dried; however, this should not injure the endothelium as in reperfusion injury. This is because 1. The bypass will have a drugline to allow its occasional wetting or medicating before and after it is reperfused, 2. A bypassed segment is usually too short to disseminate an ill effect due to the nonparticipation of the bypassed endothelium in regulating the vascular tone, 3. Normal endothelial function should be reinstated upon the resumption of blood flow, 4. At least where the ability to apply medicinal agents to the bypassed segment is concerned, no flow intervals should be brief, and 5. A brief interval of no blood flow does not induce endothelial dysfunction.

Switching between the native and synthetic lumina will usually be intermittent with neither pathway used over an extended period. The existing literature provides many references pertaining to endothelial dysfunction but not to adverse sequelae following brief and intermittent interruptions in blood flow (see, for example, Zhou, H. L., Jiang, X. Z., and Ventikos, Y. 2023. “Role of Blood Flow in Endothelial Functionality: A Review,” Online, Frontiers in Cell and Developmental Biology (Lausanne, Switzerland) 11:1259280; Chiu, J. J. and Chien, S. 2011. “Effects of Disturbed Flow on Vascular Endothelium: Pathophysiological Basis and Clinical Perspectives,” Online, Physiological Reviews (Bethesda, Maryland) 91(1): 327-387).

In compound vascular bypass organ transplantation, the meaning of bypass is distinct in pertaining to bloodlines that connect corresponding vessels of a recipient to those of a donor. Unlike bypasses which move blood to skip a segment along a single vessel and return it to the same vessel, which is intracorporeal, the bypass bloodlines in an organ transplant move blood intercorporeally.

In the repair of vascular defects small in extent and uncomplicated in position, given that druglines, side-entry jacket, valve, servovalves and nonjacketing side-entry connectors which incorporate pathophysiologically pertinent sensors can be applied to native no less than to synthetic structures to directly signal the implanted disorder response system master control microprocessor to monitor and treat the tissue, resection and end-to-end anastomosis is to be preferred to the use of a prosthesis.

Otherwise, flow diversion devices such as shunts and bypasses, as well as inline coupling jacket-secured tie-line type prostheses, consist of entry and/or exit connections which are ‘dumb’ of ‘smart’ where the inlet might be smart and the outlet passive, and a conduit joining the inlet to the outlet which can be tubular or anatomically shaped. While this structure is bipartite, neither type component is separable from the other.

2b(2)(c). Vascular Segment Prostheses: Inline Coupling Jackets and Bridging, or Tie-Lines

With a central object to provide procedures that have always and continue to require interrupting the flow of blood through the structures operated on such as in an extracardiac correction of a transposition of the great arteries addressed above in section 2b(2)(a), entitled Transectionless and ischemia-free Correction of Complete, or Dextro-transposition, of the Great arteries, and a solid organ transplant as addressed above in section 2b(1), entitled The Intercorporeal Diversion of Blood Flow, and its subsidiary sections, the elimination of cardiopulmonary bypass, cardioplegia, and general anesthesia made possible eliminates the dissectional trauma and added risks these measures introduce.

However, unlike the switchability between the bypass and anastomotic path following a bypass extracardiac correction of a transposition of the great arteries as shown in FIG. 11 or a compound vascular bypass organ transplant such as shown in FIG. 22B where the tissue is usually sound, in dealing with vascular defects, often the condition of the tissue is a direct cause for the defect. A thoracic entry wound capable of causing pain for years, and such defects usually corrected early in life, when the future integrity of the surrounding tissue is doubtful, extended replacement is a precaution to preclude the need for reentry that will preclude the need for reentry indefinitely.

described separately, inline coupling jackets and tie-lines are never used that way; a vascular prosthesis comprises entry and exit jackets to secure the tie-line connecting these to the substrate vessel stumps at either end as a unitized appliance. Measures to protect native tissue pertain to the outer half-jackets which encircle the native stumps. The need for apertures does not apply to the ductus inner half-jackets, which encircle the ends of the synthetic bridge or tie-line prosthesis.

All inline coupling jackets comprise an inlet half to grasp about the upstream stump of the substrate native vessel or ureter, and an outlet half which carries and rotates the native vessel replacement segment into position. The inlet and outlet halves of each inline coupling jacket have interposed a mechanism consisting of a miniature serrated reciprocating razor-sharp cutting edged saw to quickly transect the native vessel just before the prosthetic segment is rotated into position to replace that native. As shown in FIGS. 36A, 36C, 36E, 36H, 37A, and 37B, such a prosthesis has one entry jacket comprised of inlet and outlet halves joined and coaxially positioned for rotation of the outlet half before closure by a common spring-hinge which secures the serrated razor between the two, and one exit jacket at the exit ends of each branch and the prosthesis as a whole.

That is, each inline coupling jacket comprises two half-jackets of which the outer half jacket clutches the stumps of the native ductus while the inner half-jackets clutch the ends of the tie-line, or prosthesis, to be interposed within the substrate ductus. In a pediatric embodiment, the inline coupling double jackets are the same, or in an infant, somewhat smaller in diameter compared to those used in an adult. Inline coupling jackets accommodate growth by providing a thick inner foam lining, which compressed in proportion to growth, allows the lumina within each half to increase in diameter.

Improved Sufficiency of Seamless Prostheses Compared to Alternative Measures Such as Patches, Angioplasty, Hybrid Endovascular/Extravascular, and/or Stent Grafting

The objects in providing inline coupling jacket-connected bridging, or tie-line prostheses are first, as structural replacements for diseased or impaired ductus, larger vessels and ureters which defective, diseased, or severely damaged, must be repaired or replaced, and secondly, to much improve upon conventional means for repair such as patch aortoplasties, endovascular stent-grafts, and elephant trunks. Unlike the prostheses described herein, conventional prostheses such as stent-grafts, patch aortoplasties, and elephant and reverse elephant trunks neither expand with growth sufficiently to be placed in a neonate without the need for reentry well before adolescence, even a year later, impair the growth of the native structure beyond the repair, sometimes even the growth of the upper left extremity, and are susceptible to the development of leaks or endoleaks.

No less importantly, conventional forms of repair provide no way to directly target medication to the device. Rather, medicinal therapy which would best be targeted directly to the device instead is limited to the systemic oral routes of administration which results in dispersion throughout the circulatory system, resulting in the indiscriminate exposure of nontargeted and adversely susceptible tissue and adverse drug-drug interactions, inviting additional, essentially extraneous, adverse drug-drug and drug-food interactions and side effects.

Moreover, the use of the type prostheses described will usually be no less, indeed more medically efficacious through the direct delivery into the prosthesis of medication through a drugline, or accessory channel, shown as part number 8 or 8′ in the drawing figures, by a supportive fully implanted automatic prosthetic disorder response system as described in copending application Ser. No. 17/689,880, entitled Prosthetic Disorder Response Systems, which continuously monitors the device as well as pertinent contextual indicia in the development of the pediatric patient or an adult with one or more pertinent comorbidities and coordinates these inputs to dispense remedial medication or electrostimulation, for example, as will best instate and maintain normal homeostasis.

Additionally, the direct targeting of drugs to a localized site of disease is not bound by restrictions in the level of the dose because it would harm other tissues or organs. The initial object and no less the ensuing therapy as well as automated maintenance of the prosthesis against the buildup of clot, crystal, or infection necessarily responsive to the peculiarities of the specific type lesion, attention to the type lesions to be treated and the medical context of these is relevant.

Existing means for the repair of congenital malformities of the aorta, such as a coarctation, interruption, or aneurysm, or acquired defects such as dissections, pseudoaneurysms, and noncongenital aneurysms, consist of adapted or originally designed patient-individualized edge-stitched patch aortoplasties, which may leak; transluminally placed endovascular stent-grafts, which may leak into the space surrounding the endograft, or endoleak, subclavian patch aortoplasty, and/or hybrid repairs such as a frozen or reverse frozen elephant trunk.

Complications following these procedures such as a myocardial infarction can result from the manipulation of the affected vessel and the equipment used, which the method set forth here does not use. Others are due to shortcomings in the repair. For a repair to leak defeats the purpose of the treatment, which in the case of an aneurysm is to curtail an eventual progression in aneurysmal dilation that left untreated, would eventually result in a rupture with death imminent.

Furthermore, since a thoracic aorta neoarch tie-line type prosthesis as described herein replaces the entire segment affected, it can be used to eliminate any defect of the aortic arch regardless of its etiology or position. For this reason, repair can proceed immediately without the need for delay to plan the repair of a complex defect or at least approximate the etiological specificity where this is unclear.

In a patient with a congenital connective tissue disorder, for example, a subclavian patch aortoplasty, which otherwise receives both positive and negative reviews in the literature, is likely to use tissue no less susceptible to structural failure than that of the defect (see, for example, Allen, B. S., Halldorsson, A. O., Barth, M. J., and Ilbawi, M. N. 2000. “Modification of the Subclavian Patch Aortoplasty for repair of Aortic Coarctation in Neonates and Infants,” Online, Annals of Thoracic Surgery (Amsterdam, North Holland), Op cit., above in section 2a; 69(3): 877-881; Asano, M., Mishima, A., Yamamoto, S., Saito, T., and Manabe, T. 1998. “Modified Subclavian Flap Aortoplasty for Coarctation Repair in Patients Less than Three Months of Age,” Online, Annals of Thoracic Surgery (Amsterdam, North Holland) 66(2): 588-589.

Dehiscence, endoleaks, and migration would be dispelled by an adequately designed stitchless, seamless, leak-, and migration-proof vascular replacement conduit which rather than edge-stitched to portions of the affected native vessel at levels outside, or beyond, the defect, was secured at either end by inline coupling jackets. A patch that does not itself adequately freely expand with growth can restrain growth of the surrounding arch and although the precise character of the repair is a factor so that there is some disagreement on this point, of the left arm (see, for example, Dennis, M. R., Cusick, A., Borilovic, J., Nicholson, C., Derwin, T., Puranik, R., and Celermajer, D. S. 2019. “Left Arm Structure and Function Late after Subclavian Flap Repair of Aortic Coarctation in Childhood,” Cardiology in the Young (Cambridge, England) 29(7): 856-861; Kir, M., Ugurlu, B., Unal, N., Metin, K., Yilmaz, N., and Kizilca, O. 2015. “Revisiting Subclavian Flap Repair for Neonates and Small Infants,” Online, Pakistan Journal of Medical Sciences (Karachi, Pakistan) 31(1): 131-135; Nakanishi, K., Yokota, Y., Ando, F., Okamoto, F., Ikeda, T, and 5 others named 1992. “Late Results of the Subclavian Flap Repair for Aortic Coarctation-effects on the Left Upper Limb,” in Japanese with English abstract at Pubmed, Kyobu Geka. [Japanese Journal of Thoracic Surgery] 45(3): 204-207).

Interestingly, reports such as Dennis, et al. above of left arm undergrowth persist despite an article claiming to avoid the problem published some 33 years earlier (Meier, M. A., Lucchese, F. A., Jazbik, W., Nesralla, I. A., and Mendonça, J. T. 1986. A New Technique for Repair of Aortic Coarctation. Subclavian Flap Aortoplasty with Preservation of Arterial Blood Flow to the Left Arm,” Journal of Thoracic and Cardiovascular Surgery (St. Louis, Missouri) 92(6): 1005-1012)(see also, Talwar, S., Chandra, D., Choudhary, S. K, and Airan, B. 2015. “Repair of Coarctation of Aorta with Preservation of Blood Supply to Upper Limb,” Online, Indian Heart Journal (Amsterdam, North Holland) 67(4): 368-370).

Conventional patch aortoplasties, endoluminal stent-grafts, and hybrid forms such as frozen elephant trunks disregard the loss or disablement of the baro- and chemoreceptors in the aortic arch that normally signal the need to adjust the blood pressure in response to the level of exertion, posture, nutritional, or emotional state of the subject. The patient thus deprived of aortic or carotid bodies will adjust to the loss in blood gas partial pressure, specifically, hypoxia, hypercapnia, blood pH, and temperature reception, as well as baroreception over time, which given the higher priority of precluding a rupture, justifies disabling this sensation when unavoidable. A prosthesis of the kind described herein can incorporate pressure sensors to compensate for this loss.

Whereas a patch eliminates much of the endothelium near the origin of the systemic blood supply removing the endothelial function such as this tissue would normally contribute to include growth factors and vasoactive mediators (references above in Section 2. Background of the Invention. a. General), druglines in the inlet inline and outlet coupling jacket securing the tie-line type prosthesis or side-entry inlet and outlet diversion jackets of an aortic arch bypass conduit can release the same or equivalent agents automatically, with no need to depend upon compliance in the youngest of oldest patient, much less to take oral medication. When the loss of endothelium is extensive, prostheses and side-line entry jackets and valves where the loss with the use of a bypass is less, incorporate accessory channels for the insertion of druglines connected to drug reservoirs implanted subcutaneously in the chest which can release substances to compensate for the loss.

Both thoracic aorta and aortic arch prostheses, and aneurysm and/or coarctation bypass conduits can incorporate one or more unobtrusive piezoresistance-based thin strip strain gauge pressure sensors along the internal surface of a native or prosthetic conduit to signal the implanted disorder system microcontroller, or in comorbid disease, microprocessor, to continuously command the associated servovalve or inline coupling jackets to adjust the local pressure through the direct targeting of medication and/or by signaling the controller to divert the commensurate fraction of blood flow to another vessel, usually a large neighboring vein to achieve normotension.

The higher priority of using the lightest, smallest, simplest and least costly type of diversion coupling that will work dictates that this refinement should be reserved only for patients in whom the loss is greater and likely to prove more disturbed at a loss in chemo- or baroreception. As with conventional patches and stent-grafts, only defects too large or unsuitably shaped for repair by excision and end-to-end anastomosis are addressed. Aneurysm and/or coarctation tie-line type replacement prostheses and side-entry jacket-secured bypass conduits serve to eliminate fixation of a patch, for example, to the native aorta with stitches and the containment of the bloodstream within a seamless impermeable shell with no susceptibility to extravasating seepage. These detractions from conventional methods of repair are responsible for most long-term failures.

Risk avoidance in the thoracic aorta encompasses everything from the materials and working methods employed which must not release any debris this close to the internal carotids in particular, to providing a strength, pliancy, and resilience that thwart leakage and reasonably simulate normal function. Due to the compact apposition of the great vessels in the chest, to bypass a dissection, aneurysm, coarctation, or interruption of the thoracic aorta restricts the sizes of the inlet and to a lesser extent the outlet diversion jackets. Inline coupling jackets have no valving function and accordingly lack a sidestem 19 which even though adjustable in rotational angle and level may encroach on neighboring tissue.

In the repair of vascular defects small in extent and uncomplicated in position, given that druglines, side-entry jacket, valve, servovalves and nonjacketing side-entry connectors which incorporate pathophysiologically pertinent sensors can be applied to native no less than to synthetic structures to directly signal the implanted disorder response system master control microprocessor to monitor and treat the tissue, resection and end-to-end anastomosis is to be preferred to the use of a prosthesis.

Whether the patient is a child or an adult, replacement of most of the thoracic aorta with an inline coupling jacket-secured expandable structured prosthesis replaces congenital defects along with the adjoining tissue with a highly durable repair. If side-entry jackets or vascular valves are used to bypass the defect, abrasive encroachment upon neighboring tissue is prevented by using diversion jackets with rounded corners, edges, and external cushioning and the sidestem is optimally angled at an appropriate level.

The severe constraint on available space in the chest which prompts the use of an inline coupling jacket-secured tie-line type expandably structured prosthesis with no significant sideways protrusion would seldom apply to the abdomen where to repair a relatively common aneurysm of the aorta using a side-entry jacket-secured bypass conduit is readily achieved. Where the anatomy is compact, side-entry jackets and valves with a radially projected side stem shown as part number 19 in the drawing figures can be avoided by using an inline coupling jacket-secured aneurysm and/or coarctation replacement conduit instead. A neoarch, or inline thoracic arch replacement conduit fits into the space previously occupied by the native aortic arch.

For the present purpose, the flow characteristics of blood allow disregarding the non-Newtonian flow of whole blood which pertain to smaller vessels such as arterioles and capillaries (see, for example, Arzani, A. 2018. “Accounting for Residence-Time in Blood Rheology Models: Do We Really Need Non-Newtonian Blood Flow Modelling in Large Arteries?,” Journal of the Royal Society, Interface 15(146) 20180486; Sochi, T. 2014. “Non-Newtonian Rheology in Blood Circulation,” Physics. Fluid Dynamics, Online at https://arxiv.org/pdf/1306.2067.pdf; Huyghe, J. M., Arts; T., van Campen; D. H., and Reneman, R. S. 1992. “Porous Medium Finite Element Model of the Beating Left Ventricle,” American Journal of Physiology-Heart and Circulatory Physiology, 262(4): H1256-H1267).

The stenosis of a more pronounced coarctation can bring non-Newtonian factors into play (see, for example, Liu, H. Lan, L. Abirigo, J., Ip, H. L., Soo, Y., and 6 others 2021. “Comparison of Newtonian and Non-Newtonian Fluid Models in Blood Flow Simulation in Patients with Intracranial Arterial Stenosis,” Online, Frontiers in Physiology 12:719540 with correction 12:782647; Sriyab, S. 2020. “The Effect of Stenotic Geometry and Non-Newtonian Property of Blood Flow through Arterial Stenosis,” Cardiovascular and Hematological Disorders-Drug Targets 20(1): 16-30; Hong, H., Song, J. M., and Yeom, E. 2019. “Variations in Pulsatile Flow around Stenosed Microchannel Depending on Viscosity,” Online, PLoS [Public Library of Science] One 14(1): e0210993; Mandal, M. S, Mukhopadhyay, S., and Layek, G. C. 2012. “Pulsatile Flow of Shear Dependent Fluid in a Stenosed Artery,” Theoretical and Applied Mechanics 39(3): 209-231; Mandal, P. K. 2005. “An Unsteady Analysis of Non-Newtonian Blood Flow through Tapered Arteries with a Stenosis,” International Journal of Nonlinear Mechanics 40(1): 151-164; Shibeshi, S. S. and Collins, W. E. 2005. “The Rheology of Blood Flow in a Branched Arterial System,” Applied Rheology (Lappersdorf, Germany), Online 15(6): 398-405; Ishikawa, T., Guimaraes, L. F. R., Oshima, S, and Yamane, R. 1998. “Effect of Non-Newtonian Property of Blood on Flow through a Stenosed Tube,” Fluid Dynamics Research 22(5): 251-264; Stoltz, J. F. 1985. “Hemorheology: Significance,” Acta Medica Portuguesa 6(7-8): S4-S13; Shukla, J. B., Parihar, R. S, and Rao, B. R. P. 1980. “Effects of Stenosis on Non-Newtonian Flow of the Blood in an Artery,” Bulletin of Mathematical Biology 42(3): 283-294).

In the placement of a defect bypass, a stenosed segment is replaced by the non-constricted passageway through the conduit, eliminating non-Newtonian flow. The cross-sectional area of the prosthetic lumen equal to that of a normally formed aorta, there is no compression. The inlet diversion jacket in place and the flow of blood diverted through the conduit and outlet jacket, the defective aorta is excised.

Because conventional patching and even endoluminal repair opens the way for collateral defects outside the repair to arise, unless small enough to be correctable through excision and end-to-end anastomosis of the stumps, a defect of the thoracic arch, whether an aneurysm, coarctation, or interruption, warrants replacement with the prosthesis indicated, that is, for its ‘extension for prevention’ and seamless construction. Regardless of the etiology or whether the patient is a child or an adult, outside the repaired area, the implanted automatic disorder response system can dispense supportive medication directly to the site as necessary. Vascular wall strengthening medication is addressed above in this section.

While in an adult, an aneurysm might or might not have arisen due to one or more congenital defects but rather acquired due to atherosclerosis, hypertension, and/or following weakening of the vessel wall by a bacterial, viral, or fungal infection, the response to the extent of the surgical repair should be the same. Such malformities detectable through auscultation, sphygmomanometry, ultrasound, and/or fluoroscopic imaging, for example, the need for a prosthetic replacement in a less than fully grown patient carries with it the understanding that to avoid later problems or the need to replace it-which introduces numerous risks—the prosthesis must expand in step with growth, recommending a prosthesis made with the expandable structure addressed below in section 2b(2)(f), entitled Expandable Structure to Accommodate Growth and Self-adjust in Size in Vascular Segment Prostheses, Lines, and Confluence Conduits Placed in Children.

Between the entry and exit connecting jackets of a defect bypass used when the adjacent tissue is sound or the inline coupling jacket-secured tie-line type vascular prosthesis where it is not so that the more extensive replacement was necessary, either type of repair is formed and coated as delineated above in section 2a, entitled Background of the Invention. General, and below in section 2b(2)(f), entitled Expandable Structure to Accommodate Growth and Self-adjust in Size in Vascular Segment Prostheses, Lines, and Confluence Conduits Placed in Children, and section 6, entitled Description of the Preferred Embodiments of the Invention. Any tendency of the internal surface of the conduit to become coated with clot from the blood passing through it is countered with an occasional heparin drip automatically released into the lumen through an accessory channel of the inlet diversion jacket or inlet inline coupling jacket, the dose too slight to cause a thrombocytopenia.

A vascular prosthesis to replace a defective segment along a tubular anatomical structure or ductus comprises the synthetic prosthesis proper and the inline coupling jackets used to secure it in place at either end where as shown in FIG. 37D, the outer half-jackets are stitched entirely through the substrate native ductus with clot repellent suture, and if necessary, an occasional drop of a heparin of which the need is signaled to the master control microprocessor by an endoluminal local thin film sensor. Such a prosthesis is for interposition along a native vessel, for example, might be a synthetic tube, or catheter, a tissue engineered vessel, or an artery with or without its branches, such as the aortic arch with its supraaortic branches, the abdominal aorta to include the celiac axis, for example, or a ureter. In a pediatric patient the bridge (tie-line, prosthesis) must grow with the patient as responsive to the growth hormone flowing through it. If not, it is made as described below in section 2b(2)(f), entitled Expandable Structure to Accommodate Growth and Self-adjust in Size in Vascular Segment Prostheses, Lines, and Confluence Conduits Placed in Children.

Information pertaining to inline coupling jackets is provided below in sections 2b(2)(c)(c1), entitled Inline Coupling Jackets Proper, and section 6, entitled Description of the Preferred Embodiments of the Invention. The inline coupling jackets not only fix the bridge or tie-line as interposed along the length of the substrate vessel, but as shown in FIG. 37A, serve to effectuate the interposition. As described below in section 2b(2)(c)(c2), entitled Automatic Insertion into a Ductus of an Inline Coupling Jacket-secured Bridging, or Tie-line and Its Replacement if Necessary, the process is essentially automatic; however, the operator must be monitor the operation and be prepared to nudge it here or there to a successful positioning.

Shown in FIGS. 36A thru 36E and 37F. Inline coupling jackets and tie-lines are unitized into a vascular prosthesis of which the inline coupling half-jacket pair at either end connects the interposed prosthetic vascular replacement portion to the stump of the native ductus. Accordingly, a complete replacement thoracic aorta with supraaortic branches comprises an anatomically shaped bridging, or tie-line prosthesis, secured at either end by inline coupling jackets. Inline coupling jackets and the prostheses they fix in position represent a unification of two concepts, and in a pediatric patient three, to include a prosthesis that will expand in step with the growth of the patient, described below in section 2b(2)(f), entitled Expandable Structure to Accommodate Growth and Self-adjust in Size in Vascular Segment Prostheses, Lines, and Confluence Conduits Placed in Children.

Vascular prostheses for use in children are made using the expandable fabric described below in section 2b(2)(f), just mentioned above. Considered separately or in combination, 1. The type of prosthesis, 2. The means that allow it to expand when applicable. 3. The mechanism provided for its automatic placement, and 4. Its seamless, nonsutured, and nondehiscible fixation in position, separately, much less in combination, are all novel.

For a child with a congenital defect of the vasculature such as a coarctation, interruption, or abnormal branching of the aortic arch, or an underlying connective tissue defect that poses a constant risk of aneurysmal breakdown or dissection, the freedom to participate in physical activities and therewith the prospect of a normal childhood is eliminated. Surgical repair can eliminate the immediate defect; however, both the repair itself the underlying condition surrounding the repair will abide as a constant threat of breakdown, rupture, and death due to extravasation, or bleeding.

Most often arising along the aorta, such defects can occur anywhere along the arterial tree, and often in the legs as a popliteal or femoral aneurysm, for example (see, for example, Curi, M. A., and Rubin, B. G. 2008. “Abdominal Aortic Aneurysm: Open Repair,” pages 731-736; Malas, M. B., “Endovascular Treatment of Abdominal Aortic Aneurysms,” pages 736-742; Black, J. H. III, “Thoracic and Thoracoabdominal Aortic Aneurysms,” pages 753-761; Krupski, W. C., “Popliteal and Femoral Artery Aneurysms,” pages 765-774; in Cameron, J. L. 2008. Current Surgical Therapy, Philadelphia, Pennsylvania: Elsevier/Mosby, Safi, H. J., Estrera, A. L., Miller, C. C., III, Azizzadeh, A., and Porat, E. E. (eds.), 2008. “Thoracic Vasculature with Emphasis on the Thoracic Aorta,” in Townsend, C. M., Beauchamp. R. D., Evers, B. M., and Mattox, K. L. (eds.), Sabiston Textbook of Surgery: The Biological Basis of Modern Surgical Practice, Soutth Asia Edition, Vol. 2. Section XII. Chapter 63, pages 1853-1881; Philadelphia, Pennsylvania: Elsevier/Saunders).

Even in the absence of an underlying connective tissue disorder, rather than to replace the immediate defect with a patch aortoplasty, for example, which leaves the rest of the aorta to fail and presents seams that subject to growth would likely dehisce, and is incapable of growth to the extent that an eventual revision will be inevitable, it is fundamentally more effective to replace the thoracic aorta in its entirety with a prosthesis, which 1. Monolithic, lacks seams, 2. To preclude migration is fixed in position with the aid of through-and-through stitching to the substrate ductus of the outer half-jackets as shown in FIG. 37D, and 3. Is made with the expandable structure described in section 2b(2)(f), entitled Expandable Structure to Accommodate Growth and Self-adjust in Size in Vascular Segment Prostheses, Lines, and Confluence Conduits Placed in Children, so that placed in an infant, the prosthesis will expand in size in step with growth, and will preclude the failure seen with a delimited patch whether the breach arises along or aside from the patch.

With an aneurysm, the tissue to which the patch is stitched is usually if less noticeably defective, may be excessively elastic and/or malacotic, hence, prone to fail beyond the defect, so that the combination of a lack of strength and an inability to adapt to growth predispose to eventual dehiscence. If not periodically imaged, the consequence can be a life-threatening leak if not rupture and death. The replacement thoracic aorta described here is intended to eliminate any defective tissue to a distance beyond what a patch would cover. Viewed thus, the fact that a woven plastic patch offers versatility to the extent of obtaining a good initial fit with geometry to close the gap left following excision of the defect is not sufficient justification for its use.

Complications following patch aortoplasty have been documented (see, for example, Lv, L., Lang, X., Zhang, S., Wang, C., and Wang, Q. 2022. “Assessment of Three Types of Surgical Procedures for Supravalvar Aortic Stenosis: A Systematic Review and Meta-analysis,” Online, Frontiers in Cardiovascular Medicine (Lausanne, Switzerland) 9:987522; Price, J. D. and LaPar, D. J. 2019. “The Challenges of Redo Aortic Coarctation Repair in Adults,” Current Cardiology Reports 21(9): 99; Cramer, J. W., Ginde, S., Bartz, P. J., Tweddell, J. S., Litwin, S. B., and Earing, M. G. 2013, Op cit., above in section 2a; Chiesa, R., Melissano, G., Civilini, E., Bertoglio, L., Setacci, F., and Baccellieri, D. 2008. “Giant Aneurysm 25 Years after Patch Aortoplasty for Aortic Coarctation,” Online, Texas Heart Institute Journal (Houston, Texas) 35(2): 220-221; Santo, K. C., Guest, P., McCafferty, I., Bonser, R. S. 2007. “Aortoesophageal Fistula Secondary to Stent-graft Repair of the Thoracic Aorta after Previous Surgical Coarctation Repair,” Online, Journal of Thoracic and Cardiovascular Surgery (St. Louis, Missouri) 134(6): 1585-1586; Roth, M., Lemke, P., Schönburg, M., Klovekorn, W. P., and Bauer, E. P. 2002. “Aneurysm Formation after Patch Aortoplasty Repair (Vossschulte): Reoperation in Adults with and without Hypothermic Circulatory Arrest,” Online, Annals of Thoracic Surgery (Amsterdam, North Holland, Netherlands) 74(6): 2047-2050).

Except that the potential for expansion in step with growth when placed in a small child is limited wo that later reoperation is a virtual certainty, in terms of the means employed, a hybrid technique that incorporates an endovascular lining is clearly the best repair currently available (Lala, S., Scali, S. T., Feezor, R. J., Chandrekashhar, S., Giles, K. A., and 6 others 2018. “Outcomes of Thoracic Endovascular Aortic Repair in Adult Coarctation Patients,” Online, Journal of Vascular Surgery 67(2): 369-381.e2; Parsa, P., Eidt, J., Rios, A., Gable, D., and Vasquez, J. Jr. 2018. “Case Report: An Innovative Endovascular Technique for Repair of Descending Thoracic Aortic Aneurysm following an Open Coarctation Repair,” Annals of Vascular Surgery 46:205.e4; Baumgarten, H., Squiers, J. J., Brinkman, W. T., and Vasquez, J. Jr. 2017. “Endovascular Technique for Repair of Descending Thoracic Aortic Aneurysm after Coarctation Operation,” Annals of Thoracic Surgery 103(2): e167-e169).

Another problem encountered in reconstructions or balloon angioplasty of the aortic arch to correct a coarctation or hypoplasia is restenosis. A following repair using various organic and frozen organic materials to include allografts introduce the potential for rejection and deterioration during thawing as additional obstacles to successful treatment (see, for example, van Beynum, I. M., Kurul, S., Krasemann, T., Dalinghaus, M., deWoestijne, P. V., Etnel, J. R., and Bogers, A. J. J. C. 2021. “Reconstruction of the Aortic Arch in Neonates and Infants: The Importance of Patch Material,” Online, World Journal for Pediatric and Congenital Heart Surgery (Thousand Oaks, California) 12(4): 487-491; see also Jashari, R., Bouzet, V., Alcaraz Blanco, M. J., Oleffe A., Lecocq, E., and Mastrobuoni, S. 2023. “Vascular Allografts for Clinical Application in Europe: Assessment of 30 Years of Experience with Vascular Tissue Banking in Brussels,” Online, Cell and Tissue Banking (Dordrecht, Netherlands) January 3:1-13; Ashry, A., Harky, A. Tarmahomed, A., Ugwu, C., Mohammed, H. M., and 4 others 2022. “Contemporary Outcomes of Aortic Arch Hypoplasia and Coarctation Repair in a Tertiary Paediatric Cardiac Surgery Centre,” Cardiology in the Young (Cambridge, England) 32(7): 1098-1103; Naegeli, K. M., Kural, M. H., Li, Y., Wang, J., Hugentobler, E. A., and Niklason, L. E. 2022. “Bioengineering Human Tissues and the Future of Vascular Replacement,” Online, Circulation Research (Baltimore, Maryland) 131(1): 109-126; Novotny, R., Ericka, P., Chlupac, J., Matejka, R., Kristek, J., and 8 others 2020. “The Effect of Different Thawing Rates on Cryopreserved Human Iliac Arteries Allograft's Structural Damage and Mechanical Properties,” Online, BioMed Research International (New York, New York) 2020:6545190; Novotny, R., Slizova, D., Hlubocky, J., Krs, O., Spatenka, J., and 7 others 2017. “Cryopreserved Human Aortic Root Allografts Arterial Wall: Structural Changes Occurring during Thawing,” PLoS [Public Library of Science] One (San Francisco, California) 12(4): e0175007).

To replace a significant segment of the vasculature, such as the thoracic aorta past the aortic valve having exited the heart, hence, almost in its entirety to include its supraaortic branches and past the first two inches of the descending aorta all at once eliminates untreated portions of the structure likely to fail at any time. Furthermore, to eliminate the need for painful revision made inevitable by growth, the prosthesis to be described is made to spontaneously and continuously adjust in size in step with the growth of the patient. This capability is imparted through the use of the fabric addressed below in section 2b(2)(f), entitled Expandable Structure to Accommodate Growth and Self-adjust in Size in Vascular Segment Prostheses, Lines, and Confluence Conduits Placed in Children.

Every implant described herein permitting placement without interruption in the flow of blood through the substrate ductus and therefore making possible operations such as solid organ transplantation, carotid endarterectomy, and extracardiac transposition of the great vessels which likewise eliminate the need to interrupt the flow of blood through the substrate ductus its cardinal object, equally important is the ability imparted by the insertion mechanism addressed below in section 2b(2)(c)(c2), entitled Automatic Insertion into a Ductus of an Inline Coupling Jacket-secured Bridging, or Tie-line and Its Replacement if Necessary, to effect this replacement with no interruption in blood flow as would necessitate the use of cardiopulmonary bypass, cardioplegia, and involve a period of anoxia of around 4 hours plus, likely to result in disorientation and a measure of reperfusion injury.

Placement thus is made possible through the mechanism also described below in section 2b(2)(c)(c2) cited just above and shown in FIG. 37A. Referring to FIG. 37A, essentially, a spring-loaded serrated razor at the junction of the halves of either inline coupling jacket transects the substrate native ductus as the native ductus is rotated out of its position at the same time that the tie-line, or prosthesis, is rotated into its position. This action transects the native ductus at either end and replaces it in a continuous action such that the flow of blood through the native lumen is gradually transferred from the naïve to the prosthetic lumen. The ends of the tie-line extend beyond the half-jacket interfaces by a few millimeters, and since the flow of blood is never interrupted, the ends of the native vessel remain circular without infolds.

If and only if necessary, engagement of the ends of the tie-line into the ends of the stumps is facilitated by wetting the tie-line segment ends with liquid albumin and/or 1,2-dipalmitoyl-sn-glycero-3-phosphatidylcholine (see, for example, Su, C. Y., Yeh, L. K., Fan, T. W., Lai, C. C., and Fang, H. W. 2021. “Albumin Acts as a Lubricant on the Surface of Hydrogel and Silicone Hydrogel Contact Lenses,” Online, MDPI [Multidisciplinary Digital Publishing Institute] Polymers (Basel, Switzerland) 13(13): 2051; Yuan, H., Cheng, H. W., Mears, L. L., Huang, R., Su, R., and 3 others 2021. Lipid Anchoring Improves Lubrication and Wear Resistance of the Collagen I Matrix,” Online, [American Chemical Society] Langmuir 37(47): 13810-13815).

At the half-way point in replacement thus half the output of the left ventricle flows through the native aorta, at the same time that half flows through the prosthesis. Here the object of repairing a defective thoracic aorta, usually in a child, to remain intact to the end of life without interruption in the flow of blood is made possible; that is, a prosthesis that will spontaneously expand in step with growth and the means to insert the prosthesis without a need to interrupt the flow of blood clearly represents a significant improvement, wo While this gradual transfer of blood flow between either of two passageways represents a switching function, it is thus only once during segment insertion. For that reason, the device shown in FIG. 37A is not, strictly speaking, a valve.

The levels and landing zones separating the branch arteries originating along the aortic arch variable in length, in most cases, the length of cylindrical aorta to either side, that is, immediately upstream and downstream to the defect will be inadequate to securely fix the bypass inflow (incurrent, inlet, sending) and outflow (excurrent, outlet, receiving) basic or servovalve-jackets.

Therefore, insecure positioning and attachment to weakened tissue is avoided by positioning the bypass inflow jacket sufficiently upstream to the defect and the outflow jacket sufficiently downstream to assure sufficient length and strength for a secure fit. The bypass then carries all the blood incurrent into and excurrent out of the bypass and includes outlet branches to the arteries bypassed. In many cases, this will require positioning the inflow jacket in brachiocephalic-upstream, or landing zone 0, and the outflow jacket in left subclavian artery-downstream, or landing zone 3.

Communication from the bypass inflow to the outflow jacket is through synthetic tubing of resilience matching that of the normal aortic arch, with branches of the same material, each branch ending through a branch outflow jacket into the respective arch-originating artery. The jacket or valve jacket envelops the substrate vessel, provides an opening into the lumen which is fully contained within it, and for pulsatile compliance and to allow growth, is lined with viscoelastic foam with perforations in alignment with those through the outer jacket to preclude the atherosclerotic degeneration of the substrate vessel.

Therefore, in contrast to excision of the constriction and anastomosis, repair thus is not susceptible to recoarctation, leaking, or the formation of an aneurysm. The application of side-entry jackets or valve-jackets depends upon the location of the constriction. Three quarters of aortic coarctations associated with intracardiac defects, it is preferred that other defects be corrected by transluminal means. Because of the relative safety of switch transplantation, the fact that hypoxia leads to degradation in organs and tissues throughout the body, and improvements in immunosuppressant therapy, severe intracardiac defects such as univentricular and complex double-outlet right ventricle should be surgically ameliorated only as a bridge to transplantation.

In an infant, the presentation is usually preductal, often at the isthmus, or landing zone 3, too proximate to the left subclavian artery to place either the bypass entry or exit inline coupling jacket, which must have a bore to allow tubing of the diameter needed to initiate a normal volume and rate of normotensive flow past the defect and relieve any excess pressure preceding the constriction. Absent excision of the constriction, endostenting, or stent-grafting, retains the weakened wall of the coarctation, which should leakage occur, might result in rupture with the likelihood of death.

The constriction causes hypertension in the arteries rising from the arch, namely, the brachiocephalic trunk (innominate artery), left common carotid, and left subclavian arteries, as well as obstructs flow and reduces the pressure of blood to the lower body (see, for example, Law, M. A. and Bhimji, S. S. 2018. “Coarctation of the Aorta,” Online, Treasure Island, Florida: StatPearls Publishing Co., at https://www.ncbi.nlm.nih.gov/books/NBK430913/; Hanneman, K., Newman, B., and Chan, F. 2017. “Congenital Variants and Anomalies of the Aortic Arch,” Radiographies 7(1): 32-51; Keshavarz-Motamed, Z., Garcia, J., and Kadem, L. 2013. “Fluid Dynamics of Coarctation of the Aorta and Effect of Bicuspid Aortic Valve,” Online, PLoS [Public Library of Science] One 8(8): e 72394; Abbruzzese, P. A. and Aidala, E. 2007. “Aortic Coarctation: An Overview,” Journal of Cardiovascular Medicine (Hagerstown, Maryland) 8(2): 123-128).

In an infant with this typically configured defect, the bypass is placed to span along the path least encroaching upon neighboring tissue from the ascending arch just short of the ascending branches to just past the ductus arteriosus on the descending arch, thus alleviating the elevation in blood pressure through the three branches rising from the arch and delivering the correct volume and pressure pulsatile blood to continue down through the abdominal aorta. The caliber or gauge of the bypass connecting line is complementary to that of the constriction, thus instating normotensive flow at the proper volume to the lower body.

The bypass in place, unless the constriction is very small in diameter, the reduction in pressure approaching and passing through it with should have been sufficiently lowered to preclude the formation of an aneurysm. Resection of the constriction and end-to-end anastomosis of the free ends or the insertion of a patch often results in complications such as recoarctation and/or the formation of an aneurysm. A severe defect with small lumen is best excised with the stumps directly sutured end-to end.

Any accumulation of thrombus at the proximal or upstream cecum, or terminus, that would pose the risk of obstruction to the left subclavian and possibly left common carotid-more likely in the preductal or infantile type with less extension of the arch past the left subclavian—is prevented by directly targeting an anticoagulant such as heparin, or dissipated by direct targeting a thrombolytic to the site through an accessory channel of the bypass entry or proximal jacket (Brass, L. F. and Diamond, S. L. 2016. “Transport Physics and Biorheology in the Setting of Hemostasis and Thrombosis,” Journal of Thrombosis and Haemostasis 14(5): 906-917). In an adult, the presentation is more often postductal and sufficiently distal to the left subclavian artery to place the proximal or bypass-entry side-entry jacket along the descending arch upstream of the constriction, probably avoiding the issue of an accumulation of thrombus entering the constriction unless very small in caliber; otherwise, the process is the same as with an infantile or preductal type defect.

As with pulmonary arterial hypertension and the formation of atheromatous lesions in arteries, the body adapts to an obstruction to the normal flow of blood with increased pressure upstream and decreased pressure downstream, not just through vascular remodeling, but with the generation of collateral circulation to bypass the obstruction with a closer to normal volume of blood under less pressure (see, for example, Torok, R. D., Campbell, M. J., Fleming, G. A., and Hill, K. D. 2015. “Coarctation of the Aorta: Management from Infancy to Adulthood,” World Journal of Cardiology 7(11): 765-775; Mohsen, A., Loughran, J., and Ikram, S. 2014. “Coronary to Extra-cardiac Anastomosis,” Heart, Lung, and Vessels 6(3): 208-209; Leschka, S., Alkadhi, H., and Wildermuth, S. 2005. “Images in Cardiology. Collateral Circulation in Aortic Coarctation Shown by 64 Channel Multislice Computed Tomography Angiography,” Heart 91(11): 1422; Holmqvist, C., Ståhlberg, F., Hanseus, K., Hochbergs, P., Sandstrom, S., Larsson, E. M., and Laurin, S. 2002. “Collateral Flow in Coarctation of the Aorta with Magnetic Resonance Velocity Mapping: Correlation to Morphological Imaging of Collateral Vessels,” Journal of Magnetic Resonance Imaging 15(1): 39-46; Steffens, J. C., Bourne, M. W., Sakuma, H., O'Sullivan, M., and Higgins, C. B. 1994. “Quantification of Collateral Blood Flow in Coarctation of the Aorta by Velocity Encoded Cine Magnetic Resonance Imaging,” Circulation 90(2): 937-943).

However, with a severely constricted, or coarcted, aorta in a neonate or young child, the generation of collaterals does not progress quickly enough to assure uninterrupted circulation and therefore, normal development while collateral circulation continues to develop. Further, collaterals tend to be too small in caliber to compensate for the impediment to unrestricted blood flow. In some cases, the spontaneous development of collateral circulation never does achieve circulatory sufficiency, leaving the child with developmental delay if not irreversible deficits to include cognitive (see, for example, Moola, F., McCrindle, B. W., and Longmuir, P. E. 2009. “Physical Activity Participation in Youth with Surgically Corrected Congenital Heart Disease: Devising Guidelines So Johnny Can Participate,” Paediatrics and Child Health 14(3): 167-170).

In some instances, the consequences of a want of adequate collateral circulation, a faulty repair, or subsequent degradation in the collateral circulation that had formed, is so severe as to have resulted in paraplegia (Chiu, F. H., Tsai, S. H., and Ho, C. H. 2017. “Chest Pain and Sudden-onset Paraplegia at the Emergency Department: An Uncommon Presentation,” American Journal of Case Reports 18:728-732; Tsai, Y. D., Hsu, C. W., Hsu, C. C., Liao, W. I., and Chen, S. J. 2016. “Paraplegia Caused by Aortic Coarctation Complicated with Spinal Epidural Hemorrhage,” American Journal of Emergency Medicine 34(3): 680.e1-2; Lam, C. R. and Arciniegas, E. 1973. “Surgical Management of Coarctation of The Aorta with Minimal Collateral Circulation,” Annals of Surgery (Philadelphia, Pennsylvania) 178(6): 693-697; Adams, H. D. and van Geertruyden, H. H. 1956. “Neurologic Complications of Aortic Surgery,” Annals of Surgery (Philadelphia, Pennsylvania) 144(4): 574-610).

Adams and van Geertruyden specifically recommend “ . . . some type of shunting procedure to supply blood to the distal aorta, thereby minimizing the danger of cord damage and cardiac strain,” While appropriate in 1956, previous means of shunting, such as to suture a length of Tygon® at either end, do not provide a direct path to target medication to the treatment site such as does the accessory channel or channels in the side-entry jacket and valve-jacket connectors, are not adaptable to growth and strenuous exercise, and are susceptible to dislodgement, migration, disconnection and leaks, to which a shunt or synthetic collateral as bypass connected by side-entry jackets or valve-jackets is not.

As pertains to all of the procedures addressed herein to include vascular bypass, or switched-organ, transplantation and the extracardiac correction of complete, or dextro-transposition, of the great arteries, the central object is to eliminate the need to interrupt the flow of blood through the affected vessel as a cause of complications both mid- and postoperatively which due to their controlled shunting and bypass capabilities vascular servovalves render needless.

To this end, the mechanism whereby the native ductus is transected at either end and the prosthesis rotated around to fill its position is described below in section 2b(2)(c)(c2), entitled Automatic Insertion into a Ductus of an Inline Coupling Jacket-secured Bridging, or Tie-line and Its Replacement if Necessary, and shown in FIGS. 37A thru 37F. In contrast to servovalves, inline coupling jackets are static flow noncontrollable connectors used to secure a vascular segment prosthesis between the stumps of the removed segment, which unlike any other form of repair, incorporate sensors and accessory channels that allow the insertion of druglines so that the implanted controller or control microprocessor can instantly detect the need for and cause appropriate medication to be directly targeted to the affected site.

Attached at either end of the tie-line, each inline coupling jacket is bipartite comprised of two halves between which is a miniature serrated razor that transects the native vascular segment to be replaced. Section 2b(2)(c)(c1), below entitled Inline Coupling Jackets Proper delineates their parts and surfaces. Briefly, the upstream half of the entry jacket grasps about the substrate native vessel short of the level at which the razor between the jacket halves will transect the native vessel. The downstream half of the entry jacket grasps about the upstream end of the prosthesis. At the downstream end of the prosthesis, the halves of the outlet inline coupling jacket constitute the mirror image antipode of the upstream end.

### Insertion of a Tie-Line Prosthesis to Replace a Segment of a Native Ductus

Referring to FIG. 37A, the mechanism used to initiate the descent of the inner inline coupling half-jackets which secure the ends of the prosthesis is described below in section 2b(2)(c)(c2), entitled Automatic Insertion into a Ductus of an Inline Coupling Jacket-secured Bridging, or Tie-line and Its Replacement if Necessary. Shown as a simple linear replacement segment, the elevated tie-line can also be an anatomically shaped prosthesis. For placement in a child, the structure of the tie-line is in accordance with the description provided below in section 2b(2)(f), entitled Expandable Structure to Accommodate Growth and Self-adjust in Size in Vascular Segment Prostheses, Lines, and Confluence Conduits Placed in Children.

The ends of the tie-line extend beyond the inner half-jackets by a few millimeters. Beginning from the elevated position shown, the inner half-jackets gradually descend so that serrated razors affixed at their outer interfaces with the outer half-jackets begin to reciprocate and transect the ends of the native ductus as the tie-line prosthesis continues to descend into the position of the native segment ends become engaged in the stumps.

Because the flow of blood through the substrate native vessel is uninterrupted and the ends of the tie-line extend beyond the half-jacket interfaces, the continued flow prevents the edges of the stump ends from infolding or collapsing into their lumina. Continued descent of the tie-line gradually transfers the flow of blood from the native vessel to the tie-line, the sum volume of flow constant. If the operator has any reservations concerning the sufficiency of the joint between the ends of the tie-line and the end of the stumps, the former can be lubricated with albumin and/or 1,2-dipalmitoyl-sn-glycero-3-phosphatidylcholine. Along with the circularity imposed by closure of the half-jackets, the stump ends enter despite any minor projections of a vessel stump wall into the lumen, and closure of the jackets firmly engages the stumps.

As these ends descend the ends of the tie-line prosthetic replacement segment push down on the ends of the stumps and sweep over the native lumina so that the flow of blood is gradually transferred from the native stumps into the tie-line. When the tie-line is aligned to the native stumps, the ends of the tie-line are inserted into the ends of the native stumps. When the tie-line is made with the expansion structure, the ends of the tie-line should be flats rather than vaults. This action is always done under the watchful eye of the operator who can manually assure proper engagement of the ends of the tie-line in the ends of the native stumps.

As shown in FIG. 37D, once in position, the tie-line with inline coupling jackets connecting it to the substrate ductus at either end is fixed in position to prevent migration and dehiscence by means of stitching together the outer half-jackets of both the entry and exit, or reentry, jackets. By through-and-through stitching is meant that the clot resistant coated suture is passed entirely through the native stumps in the outer half-jacket at either end of the prosthesis overall—that is, the inlet half-jacket of the entry jacket, part number 199, and the outlet half-jacket of the exit jacket, part number 199′. The few turns of suture that traverse the lumen of the stump at either end are clot resistant, and if necessary as signaled by a local endoluminal thin film sensor, a drop of an anticoagulant, typically a heparin, is released into the lumen through drugline 8 strategically positioned just upstream to the suture 232

The halves of the double jacket are joined along one side where the jacket halves meet by a rotating joint-divided axle which courses across both with a rotatory joint at their junction and a spring that causes the inner halves to descend at the same time that the miniature serrated reciprocating saw transects the substrate native vessel. The rotary joint allows the inner halves to rotate separately, the half of the inner half-jackets incorporating a spring that when released by a vibration prompted trip urges the serrated razor of each inner-jacket to descend.

As shown in FIG. 37A, the inner, or tie-line end-grasping jacket halves, rotate the initially elevated tie-line down into alignment with the native stump, the force of the axle spring causing the serrated razor to cut through the ends of the substrate native ductus, aligning the tie-line with the ductus. The automatic mechanism is described in greater detail below in section 2b(2)(c)(c2), entitled Automatic Insertion into a Ductus of an Inline Coupling Jacket-secured Bridging, or Tie-line and Its Replacement if Necessary.

Tie-line, bridge, and prosthesis synonymous, once the prosthesis has been brought into alignment with the stump ends of the native ductus, the ends of the tie-line engage the ends of the stumps, whereupon through-and-through stitching at right angles through the outer half-jackets at either end of the tie-line as shown in FIG. 37D prevents migration. The stitching enters the inlet half of the entry and outlet half of the exit jackets through the side to emerge through the opposite side, is wound around the body of the half-jacket by 90 degrees to enter at a moderate distance from the first stitch through the top and exit through the bottom of the jacket, and so on, to fix the jackets in place.

As specified above in this section, the ends of the tie-line replacement segment slightly overextend the interfacial surface of the inner half-jackets. FIG. 37C shows that the serrated reciprocating razor saw used to transect the native ductus precedes the tie-line in descent, so that the saw cannot cut into the overextension of the tie-lines. Upon reaching the end of the downward rotation of insertion whereby the tie-line stump ends enter into alignment with the native stumps, these form a sealed joint, with leakage further prevented by the foam lining of the half-jackets and the slight compression of the half-jackets when closed.

Once the descent as indicated in FIG. 37A has concluded, the hinge common to each half-jacket pair a shown in FIG. 36C as part number 228 and the half-jacket snap shut locks maintain the half-jackets in flush relation. The joints between the tie-line and native vessel at either end of the tie-line are seamless. FIG. 37D shows the stitching employed to fix the outer half-jackets, and therewith, the inner half-jackets and tie-line in position so that it will neither dehisce nor migrate. While slight should it occur at all, if midprocedurally some seepage of blood occurs, additional hemostasis, or leakage, prevention is accomplished by wrapping about of the half-jacket interfaces with a highly adherent elastic multi-crosslinking hybrid network hydrogel tape as specified above in section 2b(1), entitled The Intercorporeal Diversion of Blood Flow. The foregoing combination of measures assures that there are no potentially dehiscible seams.

Once placed, the inline coupling jacket-secured tie-line prosthesis, made as delineated below in section 2b(2)(f), entitled Expandable Structure to Accommodate Growth and Self-adjust in Size in Vascular Segment Prostheses, Lines, and Confluence Conduits Placed in Children, is meant to remain in place to the end of life. If necessary, it can, however, be straddled by a longer inline coupling jacket-secured prosthesis placed no differently than the original. The process used in placing the prosthesis irreversible, removal otherwise requires cardiopulmonary support while the prosthesis is excised and replaced with a length of a synthetic, transplanted, or tissue engineered artery, vein, or ureter, for example.

Whether the replacement or prosthetic segment is a branchless tubular tie-line as shown in FIG. 36A with the expandable structure for placement in a small child or branched as shown in FIG. 36F, the outlet half-jacket of the entry jacket, inlet half-jacket of the exit jacket, and the intervening tie-line comprise the synthetic central portion of the prosthesis, that is, the inner half-jackets clutching the tie-line or prosthesis at either end. This and the periductal wrapping about of the inline coupling jackets once closed block any path a seepage of blood might take precluding the development of leaks.

A vascular prosthesis or simple cylindrical tie-line in a pediatric embodiment is made with the expandable structure described below in section 2b(2)(f), entitled Expandable Structure to Accommodate Growth and Self-adjust in Size in Vascular Segment Prostheses, Lines, and Confluence Conduits Placed in Children, which is designed to respond to the increased pressure of growth by expanding in both length and in caliber. The double half cylindrical inline coupling jackets at either end do not expand in length. Rather, the thick viscoelastic polyurethane parylene-coated foam lining the outer half-jackets clutching the native stumps and the inner half jackets at either end clutching the ends of the tie-line yield to the increase in stump diameter.

Accordingly, each component of the prosthesis and the inline coupling jackets securing it in position accommodates growth without potential choke points. Any means for resolving a serious vascular defect in the middle chest necessitates a midsternal, hemiclamshell, or clamshell incision, and incision into the chest causes much peri- and postoperative pain which can last for years and medication can only moderate (Zhang, J., Liu, T. X., Wang. W. X., Zhou, S. Z., Ran, X., and He. P. 2023. “Effects of Ultrasound-guided Erector Spinae Plane Block on Postoperative Acute Pain and Chronic Post-surgical Pain in Patients [Who] Underwent Video-assisted Thoracoscopic Lobectomy: A Prospective Randomized, Controlled Trial,” Online, BioMed Central Anesthesiology (London, England) 23(1): 161; Goto, T. 2018. “What is the Best Pain Control after Thoracic Surgery?,” Online, Journal of Thoracic Disease (Hong Kong, China) 10(3): 1335- 1338).

Here spontaneous expansion of the prosthesis with growth spares the patient the apprehension, additional trauma of revision, susceptibility to complications, and probably the need for opiates. The consequences of losing the aortic bodies are addressed above in section 2a, entitled Background of the Invention. General. Essentially, the deficits which result are more and more compensated for over time. A thin film pressure sensor lining the tie-line used to signal the controller of an increase in activity level can serve as a useful ‘aortic body’ if inferior to the real thing.

Later endovascular, or trans-catheter, repair dispels much pain but not the distress associated with having to undergo another procedure and little able to accommodate growth, is subject to dehiscence and postoperative leaking (see, for example, Wu, W. H., Huang, L. J., Pu, J. Z., Huang, X. Y., Pu, X., Ning. Y., and Wang, X. 2018. “Feasibility and Efficacy of Transcatheter Closure of Anastomotic Leakage after Aortic Surgery Using Amplatzer Vascular Plug III,” (in Chinese with English abstract at Pubmed), Zhonghua Xin Xue Guan Bing Za Zhi [Chinese Journal of Cardiology] (Beijing, China) 46(3): 203-207).

The complications associated with patch aortoplasties due to interfering with normal growth of the thoracic aorta is documented in the literature as cited above in this section. Expandability to accommodate growth serves to defer if not completely avoid the need for open surgical replacement from birth through late adolescence if not adulthood as no patch aortoplasty, elephant trunk, or frozen elephant trunk, for example, can provide.

Seamless, fixed in place with suture 232 passed entirely through the outer half-jackets at right angles as shown in FIG. 37D, and allowing replacement without interruption in blood flow, the resultant durability of repair substantially improves upon existing methods. Instead of an insufficiently durable and growth noncompliant reconstruction such as a patch aortoplasty or an elephant trunk, the defective vessel is suspected to be lacking in tensility aside from the evident defect as to justify its complete replacement.

Even the more recent “branch first” total arch replacement technique still requires clamping, interruption in blood flow, and the stitching of anastomoses (reiterates some references provided above in section 2a, entitled Background of the Invention. General; see, for example, Pu, J., Song, G., Ke. Y., Ma, X., Wu, W., and Zhang, H. 2023. “Techniques and Outcomes of Percutaneous Aortic Anastomosis Leak Closure after Frozen Elephant Trunk Procedure for Aortic Dissection,” Online, Journal of Thoracic Disease (Hong Kong, China) 15(3): 977-984; Acharya, M., Sherzad, H., Bashir, M., and Mariscalco, G. 2022. “The Frozen Elephant Trunk Procedure: Indications, Outcomes, and Future Directions,” Online, Cardiovascular Diagnosis and Therapy (Hangzou, China) 12(5): 708-721 Op cit. above in section 2a: Berger, T., Graap. M., Rylski, B., Fagu, A. Gottardi. R., and 6 others 2022. “Distal Aortic Failure following the Frozen Elephant Trunk Procedure for Aortic Dissection,” Online, Frontiers in Cardiovascular Medicine 9:911548; Geragotellis, A., Surkhi, A. O., Jubouri, M., Alsmadi, A. S., El-Dayeh, Y., and 3 others 2022. “Endovascular Reintervention after Frozen Elephant Trunk: Where is the Evidence?,” Journal of Cardiovascular Surgery (Turin, Italy) 63(4): 425-433; Gombert. A., Ketting, S., Rückbell, M. V., Hundertmark, A. K., Barbati, M. and 6 others 2022. “Perioperative and Long-term Outcome after Ascending Aortic and Arch Repair with Elephant Trunk and Open Thoracoabdominal Aortic Aneurysm Repair,” Journal of Vascular Surgery (New York, New York) 75(3): 824-832; Howard, C., Al-Tawil, M., Geragotellis, A., and Abdelhaliem, A. 2022. “Adverse Complications Frozen Elephant Trunk, Do We Have Enough Quality Data?,” Journal of Cardiac Surgery 37(11): 3863-3864; Jubouri, M., Kayali, F., Saha, P., Ansari, D. M., Rezaei, Y., and 5 others 2022. “Incidence of Distal Stent Graft Induced New Entry vs. Aortic Remodeling Associated with Frozen Elephant Trunk,” Online, Frontiers in Cardiovascular Medicine 9:875078; Kim, M. and Matalanis, G. 2022. “Branch-first Total Arch Replacement: A Valuable Alternative to Frozen Elephant Trunk in Acute Type A Aortic Dissection?,” Online, Indian Journal of Thoracic and Cardiovascular Surgery (New Delhi, India) 38 (Supplement 1): 58-63; Nakhaei, P., Bashir, M., Jubouri, M., Banar, S., Ilkhani. S., and 6 ohers 2022. “Aortic Remodeling, Distal Stent-graft Induced New Entry and Endoleak following Frozen Elephant Trunk: A Systematic Review and Meta-analysis,” Journal of Cardiac Surgery 37(11): 3848-3862; Nomura, Y., Tonoki, S., Kawashima, M., Fujisue, J., Uchino, G., and 5 others 2021. “Distal Stent Graft-induced New Entry after Total Arch Replacement with Frozen Elephant Trunk for Aortic Dissection,” Online, Annals of Vascular Diseases (Tokyo, Japan) 14(4): 362-367; Kreibich, M., Berger, T., Rylski, B., Chen, Z., Beyersdorf, F., Siepe, M., and Czerny, M. 2020. “Aortic Reinterventions after the Frozen Elephant Trunk Procedure,” Online, Journal of Thoracic and Cardiovascular Surgery 159(2): 392-399; Tian, D. H., Ha, H., Joshi, Y., and Yan, T. D. 2020. “Long-term Outcomes of the Frozen Elephant Trunk Procedure: A Systematic Review,” Online, Annals of Cardiothoracic Surgery (Hong Kong, China) 9(3): 144-151; Wu, W., Ke, Y., Zhao, H., Huang, L., and Pu, J. 2020. “Trans-catheter Closure of Aortic Anastomosis Leak after Aortic Replacement: Classifications and Techniques,” Online, Journal of Thoracic Disease (Hong Kong, China) 12(9): 4883-4891).

In a neonate with a congenital connective tissue disorder that bodes continued structural failure along the aorta, or in an otherwise normal infant with a focal symptomatic defect or one asymptomatic discovered incidentally, the tie-line extending between the inline coupling jacket at either end represents a reasonably anatomical facsimile. In contrast, a prosthesis to replace, for example, a smaller, or emergent aneurysm along a limited segment of the abdominal aorta, such as one superior to the celiac axis, or celiac trunk, or one inferior to the celiac axis but superior to the inferior mesenteric may consist of a relatively simple tube made with the expandable structure. While FIG. 37A shows a simple linear tie-line, extension into an additional dimension allows the use of additional outlet coupling jackets to include branches.

When a de facto defect is found in an infant with a connective tissue disorder that is focal, additional defects are likely to emerge and might involve branches (see, for example, Diletta, L., Enrico, R., and Germano, M. 2022. “Thoracoabdominal Aortic Aneurysm in Connective Tissue Disorder Patients,” Online, Indian Journal of Thoracic and Cardiovascular Surgery (New Delhi, India) 38 (Supplement 1): 146-156; Glebova, N. O., Cameron, D. E., and Black, J. H. 3rd. 2018. “Treatment of Thoracoabdominal Aortic Disease in Patients with Connective Tissue Disorders,” Online, Journal of Vascular Surgery (New York, New York) 68(4): 1257-1267).

For “extension for prevention,” most such prostheses will be more like the replacement thoracic aortic arch shown in FIG. 36F. As opposed to the carotids and coronaries, a brief period of ischemia of the smaller vessels in the abdomen will not result in any significant or lasting injury to the supplied organ, volume of tissue, or territory. Accordingly, small native branches can usually be clamped pending connection to a tie-line type prosthesis, for example.

The use of inline coupling jackets requires that sufficient substrate native vessel length up- and downstream be available to place the hinge-unified double half-cylinder entry and exit inline coupling jackets used to secure the tie-line, or bridge type, prosthesis between them. For this reason, lesions too close to the aortic root, for example, are unamenable of treatment thus. Also, while possible, to apply an inline coupling jacket right at a bifurcation such as that of the abdominal aorta into the common iliacs or of the latter in turn into the external and internal iliacs is best avoided as requiring modified inline coupling jackets with halves that meet flush at an angle complementary to the bifurcation as required to allow the interposition of the serrated razor used to transect the native vessel.

But this is not, however, feasible from a practical standpoint as the iliacs pass through muscle that would call for much dissection best avoided by resorting to an endoluminal repair. These reservations aside, it is possible to replace virtually the entire aorta in its entirety. This because inline coupling jackets do not require interruption in blood flow and therefore cardiopulmonary support and cardioplegia, which susceptible to complications even when used briefly, would be needed for many hours to complete such an extensive operation.

Another circumstance that calls for allowing space at either end of the outer inline coupling half-jackets is when the prosthesis is placed in a neonate. The expandability of a prosthesis conformant to the structure delineated above in section 2b(2)(f), entitled Expandable Structure to Accommodate Growth and Self-adjust in Size in Vascular Segment Prostheses, Lines, and Confluence Conduits Placed in Children, is intended to serve the patient indefinitely, even up to the end of life.

The synthetic tie-line or prosthesis is unsusceptible to disease but the upstream and downstream vessel is not, so that numerous conditions justify ‘extension for prevention,’ concern for spread of the disease beyond the bounds of the prosthesis suppressed medicinally with the types of drugs specified below in this section. Such conditions systemic, administration of the counteractants is best systemic, the vasculature downstream of the prosthesis directly targetable through a drugline inserted into an accessory channel of the inlet coupling jacket securing the prosthesis.

The preference for polyetheretherketone or polypropylene as the materials of which the expandable structure of the prosthesis is made is based on their high intrinsic resistance to resorption, and to this is applied a protective vapor-deposited, or sputter, coating of parylene paraxylene polymer to ward off structural degradation due to hydrolytic and enzymatic breakdown. These precautions notwithstanding, should for any reason it eventuates that the prosthesis would best or must be replaced, the method for doing so without interrupting the flow of blood through the vessel, calls for straddling the existing tie-line type prosthesis-essentially treating the original prosthesis no differently than the native vessel originally replaced. This requires that sufficient space up- and downstream of the original prosthesis be left to place the inline coupling jackets.

Whether reduced in length to leave sufficient space to replace an original prosthesis should this become necessary ab initio or extended in length to replace susceptible tissue, this is done on the basis of an assessment as to the structural sufficiency of the tissue outside the prosthesis. Significantly, both original placement and replacement are performed without interrupting the flow of blood through the native vessel. This factor is beneficial in eliminating the need for cardiopulmonary support, cardioplegia, ischemia-reperfusion injury, or general anesthesia, each alone capable of causing serious complications and reducing the quality of the outcome.

More specifically, a replacement thoracic aorta wherewith conventional treatment calls for clamping, suturing, or handling of the supra-aortic branches whether normal or presenting any of the fifteen or so more common anomalies risks the inducement of Q T segment prolongation and ventricular fibrillation. Moreover, the operation completed, the prosthesis itself, such as a patch aortoplasty, is inferior in posing seams, and in a child who is the usual patient, a lack of the ability to expand with growth thus assuring future problems and the need for reoperation.

A tie-line type neoarch will usually include supra-aortic branch terminal exit jackets actuated by the controller in synchrony with the tie-line entry and exit jackets which accomplish the connections needed with little manipulation and the least disruption. That all side-entry and inline coupling jacket-secured devices incorporate sensors to signal a problem to the system controller and accessory channels to allow the automatic targeting to the site of the medication specified in the system prescription-program is critically superior to any existing repair.

The process of replacement is described below in section 2b(2)(c)(c2), entitled Automatic Insertion into a Ductus of an Inline Coupling Jacket-secured Bridging, or Tie-line and Its Replacement if Necessary. Briefly the procedure involves straddling, that is, to bridge or span over, the original prosthesis to include the tie-line and inline coupling jackets to either side, then use the same apparatus and program as was used to automatically place the original prosthesis.

In some cases where disease had spread to branches, the replacement prosthesis is to incorporate these as well Vascular or ureteral prostheses of tie-line type are described with applicable terms below in sections 2b(2)(c)(c1), entitled Inline Coupling Jackets Proper; 2b(2)(c)(e2), entitled Automatic Insertion into a Ductus of an Inline Coupling Jacket-secured Bridging, or Tie-line and Its Replacement if Necessary; and section 2c, entitled Ductus Side-entry Jackets and Vascular Valves in Urinary Diversion.

The prosthetic thoracic arch with branches shown in FIG. 36F is shown with three branches, hence, requiring one inlet and four outlet jackets, hence, five inline coupling jackets in total, all five of which are actuated synchronously or should experience warrant, with a slight time lag for blood flow by the implanted microcontroller if the condition is monomorbid or the master control microprocessor if the condition is a comorbidity. Even in a nearly full replacement aorta in a Type IV Ehlers-Danlos patient, the length of the prosthesis is considerable, the time for blood to transit over the distance from end to end should be small enough to be disregarded.

Birth defects in fetuses result from mutations intrinsic or acquired such as those mutagenic or physiologically deleterious handed down by the mother. Broadly, birth defects are genetic, epigenetic, or a combination of both. Placement of a prosthetic disorder response system in a gravida makes it possible to directly target medication to the sites, niduses, or seedbeds, of disorders or a disease in both her and her fetus, directly or through the placenta. Tiny oximeter sensors placed at strategic sites can signal a lack of adequate oxygenation, for example. Such placement responds to informed diagnostic judgment where the potential consequences justify intervention thus. Genetic, environmental, and toxicological factors, can cause different type defects to include one or more inborn errors of metabolism.

Metabolic defects such as affect the pancreas can result from mispositioning of the fetus during gestation, to result in a disorder that remains with the neonate throughout life (see, for example, Bale, J. R., Stoll. B. J., and Lucas, A. O., [Institute of Medicine (US) Committee on Improving Birth Outcomes:] editors 2003. “Reducing Birth Defects: Meeting the Challenge in the Developing World,” Washington, D. C.: National Academies Press, Online at https://www.nebi.nlm.nih.gov/books/NBK222074/; National Institutes of Health 2017. “What Causes Brith Defects?,” Online at What causes birth defects? |NICHD—Eunice Kennedy Shriver National Institute of Child Health and Human Development (nih.gov)).

Improved Medical Sufficiency of Medication Immediately Pipe-Targeted into the Anatomical Nidus or the Tie-Line Serving it

Inborn errors in metabolism are usually associated with an inherited or a random genetic mutation, or result from improper maternal nutrition or the use of controlled or otherwise harmful substances such as alcohol or tobacco, or as secondary to a malpositioning of the fetus in utero which resulted in endocrine dysfunction due to hypoxia or interference with the normal function of an organ such as the liver or pancreas. Some metabolic consequences can be ameliorated during early development, eliminating these as complications in postpartum intervention.

Where applicable, corrective or reconstructive surgery is the more effective the earlier in development it is accomplished. Some surgical procedures to achieve normal metabolism or body structure can be performed in a neonate, sometimes, even in a late-term fetus. Where correction in utero calls for the use of drugs, once detected, providing the gravida with a fully implanted automatic ambulatory prosthetic disorder response system will automate immediate diagnosis and treatment, often before the subject is even aware of symptoms.

The utility of a disorder control system implanted in a gravida, her fetus, and placenta in the finer diagnosis and treatment of intrauterine growth restriction is addressed above in sections 2a, entitled Background of the Invention. General: 2a(1), entitled Congenital Defects of the Heart and Other Organs—the Importance of Prompt Intervention; as well as in this section.

Intrauterine malnutrition, malposition of the fetus or any of several other possible causes for intrauterine hypoxia or interference with normal organ secretive function, for example, have been documented (see for example, Orzel. A., Unrug-Bielawska, K., Filipecka-Tyczka, D., Berbeka, K., Zeber-Lubecka, N., Zielinska, M., and Kajdy, A. 2023. “Molecular Pathways of Altered Brain Development in Fetuses Exposed to Hypoxia,” Online, MDPI [Multidisciplinary Digital Publishing Institute] International Journal of Molecular Sciences (Basel, Switzerland) 4(12): 10401; Valentini, F., Rocchi, G., Vespasiani-Gentilucci, U., Guarino, M. P. L., Altomare, A., and Carotti, S. 2022. “The Origins of NAFLD [non-alcoholic fatty liver disease]: The Potential Implication of Intrauterine Life and Early Postnatal Period” Online, MDPI [Multidisciplinary Digital Publishing Institute] Cells (Basel, Switzerland) 11(3): 562; Collins, K. A. and Popek, E. 2018. “Birth Injury: Birth Asphyxia and Birth Trauma,” Online, Academic Forensic Pathology (Calgary, Alberta, Canada) 8(4): 788-864; Mohan, R., Baumann, D., and Alejandro, E. U. 2018. “Fetal Undernutrition, Placental Insufficiency, and Pancreatic β-cell Development Programming in Utero,” American Journal of Physiology. Regulatory, Integrative, and Comparative Physiology ((Bethesda, Maryland)) 315(5): R867-R878; Wilcox, G. 2018. “Impact of Pregnancy on Inborn Errors of Metabolism,” Online, Reviews in Endocrine and Metabolic Disorders (Heidelberg, Germany) 19(1): 13-33; Boehmer, B. H., Limesand, S. W., and Rozance, P. J. 2017. “The Impact of IUGR [intrauterine growth restriction] on Pancreatic Islet Development and β-cell Function,” Online, Journal of Endocrinology (Bristol, England) 235(2): R63-R76; Devaskar, S. U. and Chu, A. 2016. “Intrauterine Growth Restriction: Hungry for an Answer,” Online, Physiology (Bethesda, Maryland) 31(2): 131-146; Rainaldi, M. A. and Perlman, J. M. 2016. “Pathophysiology of Birth Asphyxia,” Clinics in Perinatology (Philadelphia, Pennsylvania) 43(3): 409-422; Gatford, K. L. and Simmons, R. A. 2013. “Prenatal Programming of Insulin Secretion in Intrauterine Growth Restriction,” Online, Clinical Obstetrics and Gynecology (Hagerstown, Maryland) 56(3): 520-528).

In an artery having already undergone aneurysmal breakdown, the composition of the arterial wall will have already been altered in such a way that a loss in endothelial function, oxidative stress, smooth muscle cell apoptosis, inflammation, and/or extracellular matrix degeneration, along with any of several genetically identified disorders are known to facilitate its continued breakdown (paraphrased after references)(DeRoo, E., Stranz, A., Yang, H., Hsieh, M. Se. C., and Zhou, T. 2022. “Endothelial Dysfunctions in the Pathogenesis of Abdominal Aortic Aneurysm,” Online, Biomolecules [Multidisciplinary Digital Publishing Institute, Basel, Switzerland] 12(4): 509; Kuivaniemi, H., Ryer, E. J., Elmore, J. R., and Tromp. G. 2015. “Understanding the Pathogenesis of Abdominal Aortic Aneurysms,” Online, Expert Review of Cardiovascular Therapy 13(9): 975-987).

Depending upon the specific condition, when progression of the malacotic condition is uncertain, the segment of the defective vessel already affected can be replaced, with medication administered to retard progression of the condition steroidal, nonsteroidal anti-inflammatoires, antihypertensives, and statins (see for example, Mayo Clinic 2023. “Thoracic Aortic Aneurysm—Diagnosis and Treatment,” Online at Thoracic aortic aneurysm—Diagnosis and treatment; “Claridge, M., Hobbs, S., Quick, C., Day, N., Bradbury, A., and Wilmink, T. 2005. “Nonsteroidal Antiinflammatory Drugs are Associated with Increased Aortic Stiffness,” Online, Vascular Health and Risk Management (Auckland, New Zealand) 1(2): 149-153).

In this, the automatic pipe-targeting through the drugline or druglines leading into the prosthesis accessory channel or channels controlled by an implanted prosthetic disorder response system facilitates prescription-adherent drug administration, especially advantageous in the very young and the very old. A neonate with a connective tissue disorder such as one of the 13 or 14 distinct genetic subtypes included in the Ehlers-Danlos syndrome, each of which affects different vessels, may be born with a serious vascular defect. The Marfan and Loeys-Dietz syndromes can also produce defects such as an aortic aneurysm.

From the standpoint of the need for repair to be extended beyond the borders of the focal lesion, the cause of any such defect is invariably ascribable to weakness in the wall surrounding the lumen, hen affecting major vessels—even the entire aorta—any of these defects can be treated by replacing the vessel with a prosthesis of the kind described in this section with subsections and below in section 2b(2)(f), as well as below in section 6, entitled Description of the Preferred Embodiments of the Invention.

Moreover, compared to the potential complications and trauma of conventional repair, the severity of the operation is fundamentally less, and can be used with infants that could not withstand a conventional repair, this averting any maldevelopment that might have obtained were the surgery deferred with a prosthesis not likely to require later reoperation. Born with or soon thereafter developing an aortic dissection, aneurysm, or pseudoaneurysm along the thoracic aorta where the involvement of a supraaortic branch may complicate a conventional repair, the replacement prosthesis replaces the thoracic aorta to include the supraaortic branches.

Such conditions are of interest from the standpoint of medical genetics, gene therapy, and the need for wider medicinal, ophthalmic, and orthopedic treatment. From the standpoint of the means for repair, the cellular and molecular distinctions between and among connective tissue disorders and aneurysms due to other causes are of scholarly but little practical significance. As to the surgical repair of an emergent aneurysm, unless discovered during the course of an open abdominal procedure undertaken for an unrelated purpose, and especially when detected as an incidentaloma in an infant with a known connective tissue disorder so that for the lesion to spread would be a near certainty, convention is to take no immediate action but rather monitor the condition.

Thay is, aneurysms often expanding at a slow rate if not stopping at the size reached when discovered, standard practice is to monitor, or ‘watch and wait,’ and defer surgical repair until the aneurysm is half again larger in diameter than the rest of the ductus. However, if the underlying condition is associated with continued growth of an aneurysm, a less risky means for effecting a permanent repair should elicit less hesitancy about effecting the correction without delay.

Where a connective tissue disorder is involved, the enjoinder enunciated in copending application Ser. No. 17/689,880, entitled Prosthetic Disorder Response Systems to the effect that where the means to correct the problem in a relatively safe and expeditious manner exists, deferring repair or using half-way measures are to be avoided. Early repair prevents any further expansion of the defect and any maldevelopment secondary to it.

Furthermore, when the lesion would appear not to abide by a less serious prognosis, especially with a congenital connective tissue disorder, early repair with ‘extension for prevention’ should prompt the replacement of as large a segment as the number of branches if any involved and consequent procedural duration will allow. Additionally, when having the potential to reemerge at any level along the arterial tree, the genetic basis for the condition should be addressed, which factor the fully implanted automatic disorder response system will be able to support through the targeted delivery of remedial agents well past the initial repair.

A fully implanted automatic prosthetic disorder response system can automatically administer gene, stem cell, or pharmaceutical therapy perioperatively and as long-term follow-up. Numerous drugs, some specified above in this section, are useful in averting aneurysmal breakdown or spreading whether due to a connective tissue disorder or infection. Replacement with a tie-line type prosthesis which is oblivious to infection and has at least one drugline to directly target antimicrobials into the lumen not only eliminates the need to harvest autologous tissue for reconstruction which operation carries numerous risks in its own right and is best avoided.

Operations to repair aneurysms that commence with the harvesting of other tissue should eventually be superseded (see, for example, Fukunaga N, Yoshida S, Shimoji A, Maeda T, Mori O, and 3 others 2022. “Surgical Strategy for Treating Mycotic Aneurysms of Thoracic and Abdominal Aorta and Iliac Artery: Analysis of Long-term Follow-up Data,” Asian Cardiovascular and Thoracic Annals 30(8): 906-911; Li L, Liu G, Yu B, Niu W, Pei Z, and 4 others 2022. “In Situ Repair or Reconstruction of the Abdominal Aorta-Iliac Artery by Autologous Fascia-peritoneum with Posterior Rectus Sheath for the Treatment of the Infected Abdominal Aortic and Iliac Artery Aneurysms: A Case Series and Literature Review,” Online, Frontiers in Cardiovascular Medicine (Lausanne, Switzerland) 9:976616; Uchida, N., Katayama, A., Tamura, K., Miwa, S., Masatsugum, K., and Sueda, T. 2012. “In Situ Replacement for Mycotic Aneurysms on the Thoracic and Abdominal Aorta Using Rifampicin-bonded Grafting and Omental Pedicle Grafting,” Annals of Thoracic Surgery (Amsterdam, North Holland) 93(2): 438-442; Laohapensang, K., Rutherford, R. B., and Arwom, S. 2010. “Infected Aneurysm” Online, Annals of Vascular Diseases (Tokyo, Japan) 31): 16-23).

Well away from the chest such as in the abdomen where a brief interruption in the blood supply or drainage through a smaller branch does not pose an immediate risk of a cardiac desynchronizing reaction as it would with the carotids or coronaries, a brief clamping to allow the native vessel to be sutured to the tie-line before the latter is placed so that leaks are precluded does not pose the risk of serious complications. Larger branches are integral in a seamless anatomical tie-line type prosthesis.

In a neonate, a malformity along a major vessel—the thoracic aorta in particular—may present as a circumscribed or focal structural defect produced during in utero. An isolated coarctation or an interrupted aortic arch will have been attributable to a genetic defect, one possibly caused by a maternal teratogen, but as genetic, may represent a disorder characteristic of the vasculature throughout the circulatory system, and as an anomaly of bronchial arch mesenchymal development, is commonly associated with the DiGeorge, or 22q11.2 deletion syndrome, and therewith, related defects such as conotruncal structural heart defects to include tetralogy of Fallot, truncus arteriosus, ventricular septal defects, and double-outlet right ventricle, as well as facial deformity, hypocalcemia, and T-cell immunodeficiency (Lewin, M. B., Lindsay, E. A., Jurecic, V., Goytia, V., Towbin, J. A., and Baldini, A. 1997. “A Genetic Etiology for Interruption of the Aortic Arch Type B [disruption between the left carotid and left subclavian arteries, representing 84 percent of cases],” American Journal of Cardiology 80(4): 493-497)(see also Alcantara, J. R., and Mendez, M. D. 2022. “Interrupted Aortic Arch,” Online, Treasure Island, Florida: StatPearls Publishing Co.).

With a genetically based disorder, surgical correction will often be necessary; however, in the future, when gene and cell therapy will have advanced to make interdiction prior to phenotypic emergence possible, the genetic defect will be detected and remediated prior to birth. Then the implantation of a disorder response system in the gravida to watch over her, her fetus, and the effect of each on the other will come into its own (see, for example, Guo, N., Liu, J. B., Li, W., Ma, Y. S., and Fu, D. 2022. “The Power and the Promise of CRISPR/Cas9 [clustered regularly interspaced short palindromic repeats/associated protein Cas9] Genome Editing for Clinical Application with Gene Therapy,” Online, Journal of Advanced Research 40:135-152; Lederer, C. W., Koniali, Buerki-Thumherr, T., Papasavva, P. L., La Grutta, S., and 4 others 2022. “Catching Them Early: Framework Parameters and Progress for Prenatal and Childhood Application of Advanced Therapies,” Online, Pharmaceutics 14(4): 793; Papanikolaou, E. and Bosio, A. 2021. “The Promise and the Hope of Gene Therapy,” Online, Frontiers in Genome Editing 3:618346; Bulaklak, K, and Gersbach, C. A. 2020, Op cit., above in section 2a, entitled Background. General; Doudna, J. A. 2020. “The Promise and Challenge of Therapeutic Genome Editing,” Online, Nature 578(7794): 229-236; Zuccaro, M. V., Xu, J., Mitchell, C., Marin, D., Zimmerman, R., and 11 others 2020. “Allele-specific Chromosome Removal after Cas9 Cleavage in Human Embryos,” Online, Cell 183(6): 1650-1664; Gyles, C. 2018. “The Promise of Gene Therapy,” Online, Canadian Veterinary Journal 59(6): 571-574; Ma, H., Marti-Gutierrez, N., Park, S. W., Wu, J., Lee, Y., and 26 others 2017. “Correction of a Pathogenic Gene Mutation in Human Embryos,” Online, Nature 548(7668): 413-419).

Fetal malpositioning will always necessitate surgical intervention. Until the intervention, an insufficiency of a metabolite or hormone, for example, due to the defect can be ameliorated or counteracted by implanting the gravida with a prosthetic disorder response system, which pending additional pregnancies can be explanted. However, if implantation of the system in the gravida will also allow the continued treatment of any primary or secondary reciprocal repercussions on her due to the defect in her fetus, leaving the system in place will be justified (see, for example, Zhang, H., Wang, Y., Qiu, Y., and Zhang, C. 2022. “Expanded Newborn Screening for Inherited Metabolic Disorders by Tandem Mass Spectrometry in a Northem Chinese Population,” Online, Frontiers in Genetics (Lausanne, Switzerland) 13:801447; Wilcox, G. 2018, Op cit., above in this section; Walter, J. H. 2000. “Inborn Errors of Metabolism and Pregnancy,” Journal of Inherited Metabolic Disease 23(3): 229-236).

In contrast to a defect or deficiency of structure caused by intrauterine molding or intrauterine growth restriction, for example (see, for example Moh, W., Graham, J. M., Wadhawan, I., and Sanchez-Lara, P. A. 2012. “Extrinsic Factors Influencing Fetal Deformation and Intrauterine Growth Restriction,” Online, Journal of Pregnancy 2012:750485; Komotar, R. J., Zacharia, B. E., Ellis, J. A., and Feldstein, N. A., and Anderson, R. C. 2006. “Pitfalls for the Pediatrician: Positional Molding or Craniosynostosis?,” Online, Pediatric Annals 35(5): 365-375), a congenital aortic aneurysm is more often due to a genetic defect of connective tissue responsible for an inadequacy of strength in the structural proteins collagen, elastin, and fibronectin in the walls of larger arteries.

This condition poses a persistent risk for extended and newly emergent aneurysmal breakdown (see, for example, Stepien, K. L., Bajdak-Rusinek, K., Fus-Kujawa, A., Kuczmik, W., and Gawron, K. 2022. “Role of Extracellular Matrix and Inflammation in Abdominal Aortic Aneurysm, Online, MDPI [Multidisciplinary Digital Publishing Institute] International Journal of Molecular Sciences (Basel, Switzerland) 23(19): 11078; Chang, Z., Zhao, G., Zhao, Y., Lu, H., Xiong, W., and 13 others 2020. “BAF60a [one of three subunits of an ATP-dependent chromatin remodeling complex] Deficiency in Vascular Smooth Muscle Cells Prevents Abdominal Aortic Aneurysm by Reducing Inflammation and Extracellular Matrix Degradation,” Online, Arteriosclerosis, Thrombosis, and Vascular Biology 40(10): 2494-2507; Martin-Alonzo, M., Garcia-Redondo, A. B., Guo, D., Camafeita, E., Martinez, F., and 11 others 2015. “Deficiency of MMP17/MT4- MMP [matrix metalloproteinase] Proteolytic Activity Predisposes to Aortic Aneurysm in Mice,” Online, Circulation Research 117(2): e13-e26; Xu, J. and Shi, G. P. 2014. “Vascular Wall Extracellular Matrix Proteins and Vascular Diseases,” Online, Biochimica et Biophysica Acta-Molecular Basis of Disease 1842(11): 2106-2119).

Interference and adverse interaction between the normal elaboration of tissue and intrauterine growth restriction during development can lead to lifelong defects which partially genetic, defy a cure. Its scope of function determined by a perinatologist on the basis of preliminary diagnostic, an implanted prosthetic disorder system with sensors implanted in the gravida, the fetus, and placenta can automatically diagnose and treat both subjects for an anticipatable disorder known to pose a risk of illness for the mother or defect for the fetus. Depending upon the results of a preliminary diagnosis by the perinatologist, intrauterine growth restriction that would lead to metabolical deficiencies which persist throughout life may be amenable to gene, stem cell, metabolite supplementation, or drug therapy.

Any of these in fluid form can be released through druglines leading to side-entry jackets or side-entry valves placed along supply arteries or nonjacketing side-entry connectors fastened to the relevant tissue. Connective tissue strengthening substances can be directly pipeline-targeted to a tie-line prosthesis along a segment of an affected vessel through a drugline leading into the inlet half of the inline coupling entry jacket as defined below in section 2b(2)(c)(c1), entitled Inline Coupling Jackets Proper, used to repair a previous defect of the vessel, for example.

The causes, structural development, and metabolic consequences of intrauterine restriction and other developmentally adverse perinatal conditions which can persist to the end of life have elicited much study. Destructive habits of the gravida such as the consumption of alcohol or the use of numerous drugs unprescribed or unlawful can be suppressed through the release of a substance causing more discomfort than the drug or drugs give comfort to prevent the behavior. If evocative of an equally pleasant experience and agreeable so that the abuse continues, then counter its adverse effects.

With sensors and effectors proper for the diagnosis, a gravida-placenta-fetal Bluetooth-coordinated detection and response implant system will be able to suppress the anticipated in utero adverse condition and respond to it before the gravida becomes aware of it. Unless quickly and effectively interdicted, in utero pathology will lead to developmental deficiencies or defects. Only an implanted ambulatory response system can react instantly and signal the event to the clinic.

Such a gravida-placenta-fetus coordinated control system need comprise little more than condition-pertinent sensors where those implanted in the fetus, placenta, and gravida are administered as separate arms or channels in a hierarchical control system wherein the information generated in these arms is coordinated, data reduced, and translated into the optimal therapy by the master control microprocessor implanted in the gravida.

In utero restriction often resulting in lifelong aberrations in metabolism, to minimize if not eradicate the source or source of restriction, the response system continues to monitor its consequences before, during and after medical intervention. Following delivery, if the intrauterine condition persists as one or more metabolic or endocrine dysfunctions, a master control microprocessor is implanted in the neonate along with the sensors which had been implanted in the fetus.

With the further development of gene and cell therapy, genetic causes for maldevelopment will become more amenable to treatment in general as well as through the monitoring and tartete release of drugs by a fully implanted automatic response system (see, for example, Barua, S. Pereira, E. M., Jobanputra, V., Anyane-Yeboa, K., Levy, B., and Liao, J. 2022. “3q27.1 Microdeletion Causes Prenatal and Postnatal Growth Restriction and Neurodevelopmental Abnormalities,” Online, BioMed Central Molecular Cytogenetics 15(1): 7; Bjarko, L., Fugelseth, D., Harsem, N., Kiserud, T., Haugen, G., and Nestaas, E. 2022. “Cardiac Morphology in Neonates with Fetal Growth Restriction,” Journal of Perinatology October 25 [in press]; Heuchel, K. M., Ebach, F., Alsat, E. A., Reutte, H., Mueller, A., and Hilger 2022. “Blood Pressure and Kidney Function in Neonates and Young Infants with Intrauterine Growth Restriction,” Online, Pediatric Nephrology 2 Sep. 2022 [in press]; Moen, G. H., Nivard, M., Bhatta, L., Warrington, N. M., Willer, C., and 3 others 2022. “Using Genomic Structural Equation Modeling to Partition the Genetic Covariance between Birthweight and Cardiometabolic Risk Factors into Maternal and Offspring Components in the Norwegian HUNT [Health Study Nord-Trondelag] Study,” Behavior Genetics 2022 Nov. 2 [in press]; Pan, Y., Chen, M., and Lash, G. 2022. “Role of Osteopontin (OPN) in Uterine Spiral Artery Remodeling,” Online, Placenta 126:70-75; Salmeri, N., Carbone, I. F., Cavoretto, P. I., Farina, A., and Morano, D. 2022. “Epigenetics beyond Fetal Growth Restriction: A Comprehensive Overview,” Molecular Diagnosis and Therapy 26(6): 607-626; Sanchez-Soriano, C. Pearson, E. R., and Reynolds, R. M. 2022. “The Role of Genetics in Fetal Programming of Adult Cardiometabolic Disease,” Journal of Developmental Origins of Health and Disease 13(3): 292-299; Tadese, K., Ernst, V., Weaver, A. L., Thacher, T. D., Rajio, T., and 4 others 2022. “Association of Perinatal Factors with Severe Obesity and Dyslipidemia in Adulthood,” Online, Journal of Primary Care and Community Health 13:21501327211058982; Hoffman, D. J., Powell, T. L., Barrett, E. S., and Hardy, D. B., 2021. “Developmental Origins of Metabolic Diseases,” Online, Physiological Reviews 101(3): 739-795; Lee. K. and Hardy, D. B. 2021. “Metabolic Consequences of Gestational Cannabinoid Exposure,” Online, MDPI [Multidisciplinary Digital Publishing Institute] International Journal of Molecular Sciences (Basel, Switzerland) 22(17): 9528; Lee. K., Laviolette, S. R., and Hardy, D. B. 2021. “Exposure to Delta9 Tetrahydrocannabinol during Rat Pregnancy Leads to Impaired Cardiac Dysfunction [sic] in Postnatal Life,” Online, Pediatric Research 90(3): 532-539; Rock, C. R., White, T. A., Piscopo, B. R., Sutherland, A. E., Miller, S. L., Camm. E. J., and Allison, B. J. 2021. “Cardiovascular and Cerebrovascular Implications of Growth Restriction: Mechanisms and Potential Treatments,” Online, MDPI [Multidisciplinary Digital Publishing Institute] International Journal of Molecular Sciences (Basel, Switzerland) 22(14): 7555; Zhao. X., Li, N., Jia. R., Chen, S., and Wang, L. 2021. “The Factors Affecting the Physical Development of Neonates in Pregnant Women with or without Gestational Diabetes Mellitus,” Online, PLoS [Public Library of Science] One 16(4): e0251024; Chen, J., Bacelis, J., Sole-Navais, P., Srivastava, A. Juodakis, J., and 13 others 2020. “Dissecting Maternal and Fetal Genetic Effects Underlying the Associations between Maternal Phenotypes, Birth Outcomes, and Adult Phenotypes: A Mendelian-Randomization and Haplotype-based Genetic Score Analysis in 10, 734 Mother-Infant Pairs,” Online, PLoS [Public Library of Science] Medicine 17(8): e1003305; Ghanchi, A., Derridi. N., Bonnet, D., Bertille, N., Salomon, L. J., and Khoshnood, B. 2020. “Children Born with Congenital Heart Defets and Growth Restrition at Birth: A Systematic Review and Meta-analysis,” Online, International Journal of Environmental Research and Public Health 17(9): 3056; Natale, B. V., Gustin, K. N., Lee, K., Holloway, A. C., Laviolette, S. R., Natale, D. R. C. and Hardy, D. B. 2020. “Delta9 Tetrahydrocannabinol Exposure during Rat Pregnancy Lead to Symmetrical Fetal Growth Restriction and Labyrinth-specific Vascular Defects in the Placenta,” Online, (Nature Research) Scientific Reports 10(1): 544; Grandjean, P., Prins, G. S., and Weihe, P. 2019. “Development Priority,” Online, Basic and Clinical Pharmacology and Toxicology 125 Supplement 3 (Supplement 3): 3-4; Heindel, J. J. 2019. “The Developmental Basis of Disease: Update on Environmental Exposures and Animal Models,” Online, Basic and Clinical Pharmacology and Toxicology 125(53): 5-13; Robson, A., Lash, G. E., Innes. B. A., Zhang, J. Y., Robson, S. C., and Bulmer, J. N. 2019. “Uterine Spiral Artery Muscle Dedifferentiation,” Human Reproduction 34(8): 1428-1438; Warrington, N. M. Beaumont, R. N. Horikoshi, M., Day, F. R., Helgeland, O., and 67 others 2019. “Maternal and Fetal Genetic Effects on Birth Weight and Their Relevance to Cardio-metabolic Risk Factors,” Online, Nature Genetics 51(5): 804-814; Kuo, A. H., Li, C., Huber, H. F., Clarke, G. D., and Nathanielsz, P. W. 2018. “Intrauterine Growth Restriction Results in Persistent Vascular Mismatch in Adulthood,” Online, Journal of Physiology 596(23): 5777-590; Sharma, D., Sharma, P., and Shastri, S. 2017. “Genetic, Metabolic, and Endocrine Aspect of Intrauterine Growth Restriction: An Update,” Journal of Maternal-Fetal and Neonatal Medicine 30(19): 2264-2275; Horikoshi, M., Beaumont, R. N., Day. F. R., Warington, N. M., Kooijman, M. N., and 38 others 2016. “Genome-wide Associations for Brist Weight and Correlations with Adult Disease,” Online, Nature 538(7624): 248-52; Sharma, D., Shastri, S., and Sharma, P. 2016. “Intrauterine Growth Restriction: Antenatal and Postnatal Aspects,” Online, Clinical Medicine Insights: Pediatrics 10:67-83; Salam, R. A., Das, J. K., and Bhutta, Z. A. 2014. “Impact of Intrauterine Growth Restriction on Long-term Health,” Clinical Nutrition and Metabolic Care 17(3): 249-254; Bhaia, J. and Gates A. 2013. “Immediate Metabolic Consequences of Intrauterine Growth Restriction and Low Birthweight,” in Bhatia, J., Bhutta, Z. A., and Kalhan, S. C. (eds.), Maternal and Child Nutrition: The First 1000 Days Nestle Nutrition Institute Workshop, (74): 157-164, Basel, Switzerland: Karger; Inadera, H. 2013. “Developmental Origins of Obesity and Type 2 Diabetes: Molecular Aspects and Role of Chemicals,” Online, Environmental Health and Preventive Medicine 18(3): 185-197; Ryckman, K. K., Borowski, K. S., Pankh, N. I., and Saftlas, A. F. 2013. “Pregnancy Complication and the Risk of Metabolic Syndrome for the Offspring,” Online, Current Cardiovascular Risk Resports 7(3): 217-223; Chernausek, S. D. 2012. ‘Update: Consequences of Abnormal Fetal Growth,” Online, Journal of Clinical Endocrinology and Metabolism 97(3): 689-695; Zanardo, V., Fanelli. T., Weiner, G., Fanos, V., Zaninotto, M., and 4 others 2011. “Intrauterine Growth Restriction is Associated with Persistent Aortic Wall Thickening and Glomerular Proteinuria during Infancy,” Online, Kidney International 80(1): 119-123; de Bie, H. M. A., Oostrom, K. J., and Delemarre-van de Waal, H. A., 2010. “Brain Development, Intelligence, and Cognitive Outcome in Children Born Small for Gestational Age,” Hormone Research in Paediatrics 73(1): 6-14; Cosmi, E., Visentin, S., Fanelli, T., Mautone, A. J. and Zanardo, V. 2009. “Aortic Intima Media Thickness in Fetuses and Children with Intrauterine Growth Restriction,” Obstetrics and Gynecology 114(5): 1109-1114; Valsamakis, G., Kanaka-Ganterbein, C., Malamitsi-Puchner, A. and Mastorakos, G. 2006. “Causes of Intrauterine Growth Restriction and the Postnatal Development of the Metabolic Syndrome,” Annals of the New York Academy of Sciences 192(1): 138-147; Gillman, M. W. 2005. “Developmental Origins of Health and Disease,” Online, New England Journal of Medicine 353(17): 1848-1850; Levy-Marchal, C. and Jaquet, D. 2004. “Long-term Metabolic Consequences of Being Born Small for Gestational Age,” Pediatric Diabetes 5(3): 147-153).

Whether the result of intrauterine growth restriction, mispositioning, or a genetic defect which does not impair intrauterine growth as such, pathological conditions identified in utero are treated in utero as well as following delivery. The relations between prenatal and postnatal defects can be, indeed, usually are, both elusive and complex. “Developmental adaptations of skeletal muscle in IUGR [intrauterine growth restriction] lead to increased risk of premature muscle loss and metabolic disease in later life” (Cortes-Araya, Y., Stenhouse, C., Salavati, M., Dan-Jumbo, S. O., Ho, W., and 4 others 2022. “KLB [β-Klotho, a fibroblast growth factor 21 (FGF21) co-receptor] Dysregulation Mediates Disrupted Muscle Development in Intrauterine Growth Restriction,” Online, Journal of Physiology (London, England) 600(7): 1771-1790)(see also, for example, Weatherall, E. L., Avilkina. V., Cortes-Araya, Y., Dan-Jumbo, S. O., Stenhouse, C. Donadeu, F. X., and Esteves, C. L. 2020. “Differentiation Potential of Mesenchymal Stem/Stromal Cells is Altered by Intrauterine Growth Restriction,” Online, Frontiers in Veterinary Science 7:558905).

The placement of an automatic prosthetic disorder response system in the gravida with sensors in the placenta and fetus chosen on the basis of preliminary prenatal diagnostic testing to reduce the scope of possible defects can intermittently or continuously diagnose and the system implanted, immediately treat the condition with medication timed to the symptoms. When the perinatologist identifies a deviation from normal development during pregnancy, the implantation of a disorder response system in the gravida, placenta, and fetus should follow promptly. The prenatal system sensors and effectors implanted in the fetus and placenta responsive to the master controller in the mother are kept to the known pertinent minimum with an independent master controller implanted in the neonate soon after birth.

Given the level of gene, stem cell, and pharmaceutical therapy available, due to its immediacy of response wherever the gravida is situated when symptoms are first detected, the genetic, epigenetic, and other defects such a system can counteract may well serve as a central factor in making possible a live birth as well as improved health of the baby throughout life (see, for example, Kesavan, K. and Devaskar, S. U. 2019. “Intrauterine Growth Restriction: Postnatal Monitoring and Outcomes,” Pediatric Clinics of North America (Philadelphia, Pennsylvania) 66(2): 403-423; Longo, S., Bollani, L., Decembrino, L., Di Comite, A. Angelini, M., and Stronati, M. 2013. “Short-term and Long-term Sequelae in Intrauterine Growth Retardation (IUGR),” Journal of Maternal-Fetal and Neonatal Medicine 26(3): 222-225).

The scope of prenatal defects which result in postnatal defects is very large (see, for example, Doan, T. N. A., Akison, L. K., and Bianco-Miotto T. 2022. “Epigenetic Mechanisms Responsible for the Transgenerational Inheritance of Intrauterine Growth Restriction Phenotypes,” Online Frontiers in Endocrinology (Lausanne, Switzerland) 13:838737; Posont, R. J., Most, M. S., Cadaret, C. N., Marks-Nelson, E. S., Beede, K. A., and 4 others 2022. “Primary Myoblasts from Intrauterine Growth-restricted Fetal Sheep Exhibit Intrinsic Dysfunction of Proliferation and Differentiation that Coincides with Enrichment of Inflammatory Cytokine Signaling Pathways,” Journal of Animal Science 100(8): skac145; Zuccarello, D., Sorrentino, U., Brasson, V., Marin, L, Piccolo, C., and 3 others 2022. “Epigenetics of Pregnancy Looking Beyond the DNA Code,” Online, Journal of Assisted Reproduction and Genetics (Amsterdam North Holland, Netherlands) 39(4): 801-816; Ali, A., Murani, E., Hadlich, F., Liu, X., Wimmers, K., and Ponsuksili, S. 2021. “Prenatal Skeletal Muscle Transcriptome Analysis Reveals Novel MicroRNA-mRNA Networks Associated with Intrauterine Growth Restriction in Pigs,” Online, Cells 10(5): 1007; Felicioni, F. Pereira, A. D., Caldeira-Brant, A. L. Santos, T. G., Paula, T. M. D., and 9 others 2020. “Postnatal Development of Skeletal Muscle in Pigs with Intrauterine Growth Restriction: Morphofunctional Phenotype and Molecular Mechanisms,” Online, Journal of Anatomy 236(5): 840-853; Rozance, P. J., Zastoupil, L. Wesołowski, S. R., Goldstrohm, D. A. Strahan, B., and 6 others 2018. “Skeletal Muscle Protein Accretion Rates and Hindlimb Growth are Reduced in Late Gestation Intrauterine Growth-restricted Fetal Sheep,” Online, Journal of Physiology 596(1): 67-82), relative to which a fully implanted prosthetic disorder response system as described in copending application Ser. No. 17/689,880, entitled Prosthetic Disorder Response Systems, can supplement in utero surgical correction with immediate diagnostics and therapy postnatally and as long thereafter as necessary.

The distinction between growth restrictive as such and other factors genetic or otherwise may be unclear so as to adversely affect the prescription-program assigned to an automatic disorder response system implanted in the gravida with sensors not only in her but in the placenta and fetus as well. The involvement of some congenital defects in a fetus affected by intrauterine growth restriction may call for therapy not necessarily prompted as a response to the growth restriction. Intra- and post-uterine therapy is the closer to optimized the greater is any ambiguity concerning a given phenotypical presentation clarified as to etiology.

If one or more additional components congenital or otherwise are identified, a pre- or neonatal heart transplant for example, here using a switched-organ method, could provide medicinal, gene, and/or stem cell therapeutic support responsive to the causative factors both restrictive and essentially extrinsic thereto (see, for example, Aliasi, M., Snoep, M. C., van Geloven, N. and Haak, M. C. 2022, Op cit. above in section 2a(1), entitled Congenital Defects of the Heart and Other Organs: The Importance of Prompt Intervention; Marks K J, Howards P P, Smarr M M, Flanders W D, Northstone K, and 4 others 2021. ““Prenatal Exposure to Mixtures of Persistent Endocrine-disrupting Chemicals and Birth Size in a Population-based Cohort of British Girls,” Online, Epidemiology (Cambridge, Massachusetts 32(4): 573-582; Ghanchi, A., Derridi, N., Bonnet, D., Bertille, N., Salomon, L. J., and Khoshnood, B. 2020. “Children Born with Congenital Heart Defects and Growth Restriction at Birth: A Systematic Review and Meta-analysis,” Online, International Journal of Environmental Research and Public Health 17(9): 3056; van Nisselrooij, A. E. L., Jansen, F. A. R., van Geloven, N., Linskens, I. H., Pajkrt, E., and 6 others 2020. “Impact of Extracardiac Pathology on Head Growth in Fetuses with Congenital Heart Defect,” Op cit., above in section 2a(1), entitled Congenital Defects of the Heart and Other Organs: The Importance of Prompt Intervention; Heazell, A. E., Hayes, D. J., Whitworth, M., Takwoingi, Y., Bayliss, S. E., and Davenport, C. 2019. “Biochemical Tests of Placental Function versus Ultrasound Assessment of Fetal Size for Stillbirth and Small-for-gestational-age Infants,” Online, Cochrane Database of Systematic Reviews 5(5): CD012245; Puccio. G., Giuffre. M., Piccione, M., Piro, E., Rinaudo, G., and Corsello, G. 2013. “Intrauterine Growth Restriction and Congenital Malformations: A Retrospective Epidemiological Study,” Online, [BioMed Central] Italian Journal of Pediatrics (London, England) 11:39:23).

An abdominal aortic aneurysm in an adult may indicate a genetic proclivity but is usually acquired due to atherosclerosis and/or an epigenetic factor, such as a viral, bacterial, or fungal infection, often aggravated by hypertension and/or inhaling cigarette smoke (see, for example, Faluk, M. and De Jesus, O. 2022. “Saccular Aneurysm,” Online, Treasure Island, Florida: StatPearls Publishing Co; Shaw, P. M., Loree, J., and Gibbons, R. C. 2022. “Abdominal Aortic Aneurysm,” Online, Treasure Island, Florida: StatPearls Publishing Co.). As tiny intracranial saccular, or ‘berry,’ and interlocular aneurysms at the base of the brain attest, aneurysmal breakdown is confined to neither large arteries such as the aorta, pulmonary, femoral, and popliteal, nor is it confined to arteries to the exclusion of veins.

While neighboring tissue in the brain will be exposed to the drugs, the implanted disorder response system can automatically treat an infectious intracranial aneurysm by releasing an antibacterial or antifungal through the internal carotids (see, for example, Krings, T., Lasjaunias, P. L., Geibprasert, S., Pereira, V., and Hans, F. J. 2008. “The Aneurysmal Wall. The Key to a Subclassification of Intracranial Arterial Aneurysm Vasculopathies?” Online, International Neuroradiology 14 (Supplement 1): 39-47); De Blasi, R., Medicamento, N., Chiumarullo, L., Salvati, A., Maghenzani, M., Dicuonzo, F., and Carella, A. 2009. “A Case of Aneurysm on a Persistent Hypoglossal Artery Treated by Endovascular Coiling,” Online, International Neuroradiology 15(2): 175-178).

When, as is usually the case, a focal structural defect in ab adult can be repaired endoluminally without affecting the pulsatile character of blood flow through it, the procedure is advisable. If the patient is a child, then to avert dehiscence and leakage, an expansion with growth prosthesis as addressed above in section 2a, entitled Background of the Invention. General; and below in section 2b(2)(f) entitled Expandable Structure to Accommodate Growth and Self-adjust in Size in Vascular Segment Prostheses, Lines, and Confluence Conduits Placed in Children; and section 6, entitled Description of the Preferred Embodiments of the Invention should be used.

If the cause is genetic, gene therapy should eventually become able to affect a more vascular wide amelioration of the condition than just to assist in treating the focal lesion. Given that a prosthetic disorder response system can administer not only aneurysm-counteractive drugs such as those specified above but gene therapy, it can play a significant part in preventing further arterial degeneration and breakdown.

Under study for over two decades, much further research is needed before gene injection will become an everyday perfunctory means for ameliorating an inborn predisposition toward aneurysmal breakdown (see, for example, Laurent, D., Small, C., Lucke-Wold, B., Dodd, W. S., Chalouhi, N., and 9 others 2022. “Understanding the Genetics of Intracranial Aneurysms: A Primer,” Online, Clinical Neurology and Neurosurgery (Amsterdam North Hollan) 212:107060; Lu T, Liu Z, Guo D, Ma C, Duan L, and 7 others 2022. “Transcriptome-based Dissection of Intracranial Aneurysms Unveils an “Immuno—Thermal” Microenvironment and Defines a Pathological Feature-derived Gene Signature for Risk Estimation,” Frontiers in Immunology (Lausanne, Switzerland) 31;13:878195; Adamou, A., Alexandrou., M, Roth, C., Chatziioannou, A., and Papanagiotou, P. 2021. “Endovascular Treatment of Intracranial Aneurysms,” Online, MDPI [Multidisciplinary Digital Publishing Institute] Life (Basel, Switzerland) 11(4): 335; Theodotou, C. B., Snelling, B. M., Sur, S., Haussen, D. C., Peterson, E. C., and Elhammady, M. S. 2017. “Genetic Associations of Intracranial Aneurysm Formation and Sub-arachnoid Hemorrhage,” Online, Asian Journal of Neurosurgery (Mumbai [Bombay] India) 12(3): 374-381; Ribourtout, E, and Raymond, J. 2004. “Gene Therapy and Endovascular Treatment of Intracranial Aneurysms,” Online, Stroke (Baltimore, Maryland) 35(3): 786-793).

FIG. 36A shows a side view of an inline coupling jacket-placed and secured prosthesis to serve as a guide or means of orientation for the sectional views in FIGS. 36B thru 36D that follow it, as simply cylindrical, and unlike the prosthesis shown in FIG. 36F, lacking branches. The great vessels mostly round but somewhat oblate in bends and flared in cross-section along their length, attaching the outer half-jackets as shown in FIG. 37A to the end of either stump when such a vessel is transected imposes circularity to expedite the insertion of the prosthesis ends into the jackets.

When used to replace a focal defect along a vessel susceptible to breakdown, the prosthesis is sized to replace not just the immediate section of the segment but the wider section within which it is located. That is, because after repair by conventional, that is, focused surgical means such as a patch aortoplasty or elephant trunk, lesions such as aneurysms, dissections, coarctations, and interruptions often develop beyond the borders of the original repair, extension to replace neighboring tissue will preclude any such eventuality.

However, if for any reason it is anticipated that will be a need to replace the device after a long period, the prosthesis should be made shorter to provide segments to either side in order to allow a replacement prosthesis to straddle and replace the original prosthesis. Concern for the exposed portions is then reduced in likelihood with drugs known to strengthen vascular tissue of which several are mentioned above in this section. Because vascular prostheses for patients of any age are made using the expandable structure delineated below in section 2b(2)(f), entitled Expandable Structure to Accommodate Growth and Self-adjust in Size in Vascular Segment Prostheses, Lines, and Confluence Conduits Placed in Children, it should be possible to provide these in any patient from a neonate to an adult. Dispensed as fully contracted, the operator manually pulls the prosthesis open to the length appropriate for the patient.

That the expansion feature also eliminates a need to provide any segment-specific tie line type prosthesis in different sizes also reduces the production cost, important to allow use in the developing world. Thorough coverage conforms to a principle enunciated in copending application Ser. No. 17/689,880, entitled Prosthetic Disorder Response Systems, which states that a superior repair having become available, whenever possible, half-way remedial measures such as the repair of a univentricular heart which fails to establish normal pulsatile flow allowing the continuation of poor development in a pediatric patient should be avoided.

Seamless and compliant in size with growth, the tie-line prostheses described herein will not dehisce, and stitched in place, postoperative migration is eliminated as well. The expandable structure and inline coupling jackets connecting it at either end presenting no projections as would abrade neighboring tissue, an aneurysm coursing amid muscles where the fascia between the separate muscles surrounding the vessel and the vessel can be easily teased apart should be amenable to an open tie-line prosthesis type repair. However, if much dissection is required to access the vessel, the repair is best endoluminal (see, for example, Hammond, E., Nassereddin, A., and Costanza, M. 2023. “Anatomy, Abdomen and Pelvis: External Iliac Arteries,” Online, Treasure Island, Florida: StatPearls Publishing Co.).

In patients with acquired or congenital clotting syndromes, due to the potential for clot to contribute to the spreading of aneurysmal breakdown, extension of an abdominal aortic aneurysm caudally, or inferior to, that is, to either or both branches of the iliac is best repaired with the placement of an inline coupling jacket-secured tie-line prosthesis. This so that a drugline inserted in a prosthesis accessory channel can be used to intermittently, automatically, and directly pipe-target a heparin drip, another anticoagulant, or thrombolytic to the site, if necessary, in a higher concentration than should be allowed to circulate with the risk of inducing a thrombocytopenia or another anticoagulant-induced side effect such as a warfarin-induced “ . . . intracranial hemorrhage, gastrointestinal bleeding, hematemesis, intraocular bleeding, and hemarthrosis, which can occur at virtually any site on the body,” (Patel, S., Singh, R., Preuss, C. V., and Patel, N. 2023. “Warfarin,” Online, Treasure Island, Florida: StatPearls Publishing Co.); (see also, Nicolas, D., Nicolas, S., Hodgens, A., and Reed, M. 2023. “Heparin-induced Thrombocytopenia,” Online, Treasure Island, Florida: StatPearls Publishing Co.; Wang, Y., Zhu, C., Leach, J., Gasper, W., Saloner, D., and Hope, M. 2020. “Growth of Common Iliac Artery Aneurysms Coexisting with Abdominal Aortic Aneurysms: Associated Factors and Potential Role of Intraluminal Thrombus,” Online, Quantitative Imaging in Medicine and Surgery (Hong Kong, China) 10(3): 703-712; Zhu, C., Leach, J. R., Wang, Y., Gasper, W., Saloner, D., and Hope, M. D. 2020. Intraluminal Thrombus Predicts Rapid Growth of Abdominal Aortic Aneurysms,” Radiology (Easton, Pennsylvania) 294(3): 707-713).

To the extent that the vessel is situated amid muscle, the drugline leading into the accessory channel best follows along with the artery, the routing thereof up to the drug reservoir implanted subcutaneously in the pectoral region accomplished with the aid of ultrasound scanning, multi-slice spiral computed tomography angiography, magnetic resonance angiography, or serial cross-sectional imaging (computed tomography and magnetic resonance imaging with multiplanar reconstruction (magnetic resonance imaging). In a neonate, the inline coupling jacket-secured replacement segment, or tie-line, should be made with the expandable structure delineated below in section 2b(2)(f), entitled Expandable Structure to Accommodate Growth and Self-adjust in Size in Vascular Segment Prostheses, Lines, and Confluence Conduits Placed in Children.

The durability of repair without a need for later revision is least with an expandable synthetic prosthesis indifferent to degenerative processes in living tissue. For conventionally repaired aneurysms and coarctations, recurrence and the need for later reentry is ordinary. Based upon the location and size of smaller and midsized vessels, these too can be treated with highly miniaturized inline coupling jackets and tie-lines. In older adults, durability is less a factor, so that conventional repair such as a patch, endoprosthesis, or stent-graft may be preferable. However, for use to repair a coarctation of the thoracic aorta which causes hypertension in a baby, angioplasty balloon expansion and stenting in place of an expandable prosthesis is inferior in that the need for a second procedure at a later date is a virtual certainty.

In a pediatric patient, the accommodation of growth by inline coupling jackets and tie-line type prostheses allows a better repair of smaller and midsized vessels which will grow with the same type defect likely appearing outside the area of repair (see, for example, Selim, M., Nasr, M. A., Alkouzaie, M. T., Al Noaimi, D. A., and Alam, M. H. 2020. “Incidental Unruptured Isolated Left Common Iliac Artery Aneurysm: A Rare Entity,” Online, Cureus (Palo Alto, California) 12(11): e11588; Huang, Y., Gloviczki, P., Dundan, A. A. Kaira, M., Hoskin, T. L., and 3 others 2008. “Common Iliac Artery Aneurysm: Expansion Rate and Results of Open Surgical and Endovascular Repair,” Online, Journal of Vascular Surgery (New York, New York) 47(6): 1203-1210). Dix, F. P., Titi, M., and Al-Khaffaf, H. 2005. “The Isolated Internal Iliac Artery Aneurysm—A Review,” Online, European Journal of Vascular and Endovascular Surgery 30(2): 119-121).

The lower limit to the caliber of the vessel reconstructed thus is set by the degree of miniaturization attainable in inline coupling jackets—a vessel of 3 or so millimeters in outer diameter. However, small vessels are less susceptible to structural failure and are supported by collateral circulation so that not all defects of small vessels need necessarily warrant surgical treatment. The placement of very small tie-line prostheses relatively rare, where repair is needed nevertheless, the procedure may require microsurgical equipment and training.

A factor that fundamentally distinguishes repair using a tie-line type prosthesis compared to any conventional repair is that at least the inlet inline coupling half jacket incorporates one or more accessory channels to admit a drugline. This allows the release into the prosthesis of a supportive drugs and maintenance solutions as necessary, such as a slow heparin drip to dispel the formation of thrombus not eliminated by an antithrombotic coating, or if used to treat an aneurysm, a vessel wall strengthening drug such as those specified above in this section.

Repair of an aneurysm of the popliteal artery requires not only that neighboring tissue not be abraded but that this apply when the knee is bent. Inline coupling inlet jackets for use at such a flexion point should have an accessory channel for insertion of a drugline in coaxial rather than transverse relation to the jacket. The drugline is routed subcutaneously from the drug reservoir in the chest to the level of the repair where the muscles are teased apart to admit the drugline into the jacket. In some cases, the drugline can pass alongside the affected vessel.

In an infant or small child, the expandable structure described below in section 2b(2)(f), entitled Expandable Structure to Accommodate Growth and Self-adjust in Size in Vascular Segment Prostheses, Lines, and Confluence Conduits Placed in Children, should be preferred; in an adult, one should opt for an endoluminal repair (see, for example Xiao, X., Feng, R., Wang, M., Sun, H., Jing, T., and 5 others 2022. “Comparisons of Outcomes of Open Surgery versus Endovascular Intervention for Thrombotic Popliteal Artery Aneurysm with Acute Lower Limb Ischemia: A Systematic Review,” Online, BioMed Central Surgery (London, England) 22(1): 398; Ascher, E., Markevich, N., Schutzer, R. W., Kallakuri, S., Jacob, T., and Hingorani, A. P. 2003. “Small Popliteal Artery Aneurysms: Are They Clinically Significant?,” Online, Journal of Vascular Surgery (New York, New York) 37(4): 755-760).

Endothelial cells are the innermost, or adaxial, single cell layer lining the lumen of a blood vessel. Supporting the area these cover and contributing to the sum of endothelial function, the local loss of endothelium as when a portion of the aorta, for example, must be replaced reduces the internal surface area requiring support to the same extent. Notwithstanding the function of the endothelium throughout the body as a factor in the control of blood viscosity, platelet cohesion, vascular tone, immunology, inflammation, angiogenesis, and hemostasis, in terms of the total circulatory system, such a loss is of negligible consequence, ameliorable with medication, and unlikely to exert much in the way of an adverse effect beyond the margins of the repair. When the thoracic aorta is replaced, endothelial surface area and the aortic body baroreceptor are lost. However, a tie-line type prosthesis can incorporate a pressure sensor controlled by the implanted disorder response system and receive compensatory medication through its drugline or druglines.

Nevertheless, if not compensated for, due to their diversity of function, the loss of endothelial cells and their function when the thoracic aorta with its aortic body or carotid arteries with their carotid bodies are removed to place a prosthesis is somewhat greater than proportional to the extent of the internal surface area lost. These baroreceptors are susceptible to the formation of tumors necessitating their removal. While nitrates can be released into a vascular prosthesis through a drugline and accessory channel, part number 8 in the drawing figures, ordinarily, the need therefor should be rare and then not likely due exclusively to the loss of endothelial cells in the area of the small prosthesis.

The consequences associated with the loss of chemo- and baroreception is addressed briefly in this section and below in section 2b(2)(g), entitled Ischemia and Hypoperfusion-free Carotid Endarterectomy Bypass and Prosthesis, and much the same pertains to the loss of such function following replacement of the innate thoracic aorta or portion thereof (see also Elsaafien, K., Harden, S. W., Johnson, D. N., Kimball, A. K., Sheng, W., and 5 others 2022. “A Novel Organ-specific Approach to Selectively Target Sensory Afferents Innervating the Aortic Arch,” Online, Frontiers in Physiology 13:841078; Reutersberg, B., Pelisek, J., Ouda, A., de Rougemont, O., Rossler, F., and Zimmermann, A. 2022, Op cit. above in the same section; Gu, J. G. and Berkowitz, D. E. 2020. “Tentonin 3 as a Baroreceptor Mechanosensor: Not a Stretch,” Online, Journal of Clinical Investigation 130(7): 3412-3415).

However, in many if not all lesions demanding excision, certainly aneurysms, local endothelial function had already been adversely affected (see, for example, Armstrong; M., Kerndt, C. C., and Moore, R. A. 2023. “Physiology, Baroreceptors,” Online, Treasure Island, Florida: StatPearls Publishing Co.; Feletou, M. 2011. The Endothelium: Part 1: “Multiple Functions of the Endothelial Cells—Focus on Endothelium-derived Vasoactive Mediators,” San Rafael, California: Morgan and Claypool Life Sciences, Online at Multiple Functions of the Endothelial Cells—The Endothelium—NCBI Bookshelf (nih.gov).

An inline coupling jacket-secured prosthesis can include the aorta almost in its entirety, in which case a significant proportion of the endothelium in the body is removed. Then the detection of a need for and automatic release by direct pipe-targeting of numerous supplements to simulate the multiple normal endocrine functions the endothelium provides is made possible by direct delivery through druglines into the inlet inline coupling jacket securing the prosthesis.

The endothelium affects the entire circulatory system and the entire body. The direct release into the circulation, effectively a form of infusion, is superior to oral administration, especially given the number of medicinals that may be needed (see, for example, DeRoo, E., Stranz, A., Yang, H., Hsieh, M, Se, C., and Zhou, T. 2022, Op cit. above in section 2b(2)(c), entitled Vascular Segment Prostheses: Inline Coupling Jackets and Bridging, or Tie-lines; (see also, Bianchi, F., Cappella, A., Gagliano, N., Sfondrini, L., and Stacchiotti, A. 2022. “Polyphenols-Gut-Heart: An Impactful Relationship to Improve Cardiovascular Diseases,” Online, MDPI [Multidisciplinary Digital Publishing Institute] Antioxidants (Basel, Switzerland) 11(9): 1700;) Rajendran, P., Rengarajan, T., Thangavel, J., Nishigaki, Y., Sakthisekaran, D., Sethi, G., and Nishigaki, I. 2013. “The Vascular Endothelium and Human Diseases,” Online, International Journal of Biological Sciences (Lake Haven, New South Wales, Australia) 9(10): 1057-1069. Müller, M. M. and Griesmacher, A. 2000. “Markers of Endothelial Dysfunction,” Clinical Chemistry and Laboratory Medicine (Berlin, Germany) 38(2): 77-85; Boulanger, C. M. 1999. “Secondary Endothelial Dysfunction: Hypertension and Heart Failure,” Journal of Molecular and Cellular Cardiology (London, England) 31(1): 39-49; Shimokawa, H. 1999. “Primary Endothelial Dysfunction: Atherosclerosis,” Journal of Molecular and Cellular Cardiology (London, England) 31(1): 23-37).

The internal surface of the prosthesis is made of an intrinsically anticoagulant and/or antithrombotic material and/or coated to similarly obstruct the accumulation of clot, and a drugline to release supportive medication to include an anticoagulant such as in the form of a slow heparin drip or any other anticoagulant in liquid form directly into the device is provided. Control over the release of medication into the prosthesis is encoded in the prescription-program executed by the microcontroller, or in multimorbid disease, the master control microprocessor.

To the extent that the prosthesis has the elasticity to respond to changes in blood pressure, which in a normotensive patient comprehends a relatively small range of values, endothelial and endocrine-like mediated support is imparted by the passive elasticity of the device attributable to its material and structure. Metabolites, enzymes, hormones, drugs, and combinations of these can be promptly dispensed according to the system prescription-program. FIGS. 26A thru 26C and 27A thru 27C show means which allow the quick replenishment of these substances in the system drug reservoirs.

For use in a neonate, infant, or small child, the prosthesis—such as an aortic neoarch for placement in a neonate, is made as specified below in section 2b(2)(f), entitled Expandable Structure to Accommodate Growth and Self-adjust in Size in Vascular Segment Prostheses, Lines, and Confluence Conduits Placed in Children, is semiautomatically placed as described below in section 2b(2)(c)(c2), entitled Automatic Insertion into a Ductus of an Inline Coupling jacket-Secured Bridging, or Tie-line and Its Replacement if Necessary, and fixed in position as described above in this section and below in the section that follows entitled 2b(2)(c)(c1), entitled Inline Coupling Jackets Proper.

The tie-line or prosthesis structure is configured to expand with growth while at the same time pose a limited resilience essential to accommodate and assist in propelling the pulse through the advancing column of blood without irritating neighboring tissue. Essentially automatic, the mechanism used for tie-line or vascular segment prosthesis placement, shown in the raised position in FIG. 37A, is rotated into the position of the native ductus; while automatic, it is, nevertheless intended to be used under the watchful eye of the surgical operator who can interrupt and correct the action at any moment if necessary. The circumferential elevations, or vaults, which progressively open to flatten, readily compress until fully expanded due to growth and prior to full extension, actually serve as pads against neighboring tissue.

Especially through the abdomen which lacks sternum and full ribcage shielding, an expandable prosthesis must also provide an end-tensile strength (failure strain, bursting force) sufficient to withstand a direct impact. The interior of the prosthesis is surface treated to dispel biofilm and clot, and the drugline 8 to the inlet half of the entry jacket as defined below in section 2b(2)(c)(c1), entitled Inline Coupling Jackets Proper, is available to release supportive agents as specified in the implanted automatic response system prescription-program.

Thus, rather than using a conventional reinforced elastic fabric lacking in the other essential physical properties and incapable of the degree of extension required, needed to securely endure from infancy to at least adolescence and best to adulthood and thereafter, such as the Dacron® polyester (polyethylene terephthalate) used in a patch aortoplasty, for example, the immediate repair allows a frail neonate to develop normally as well as to gain the strength to tolerate a revision in the unlikely event that such becomes necessary.

An additional benefit for neonates and infants with severe and difficult to repair malformities of the thoracic aorta is that compared to surgical repair, which traumatic is unlikely to provide durability much less adapt to growth, an expandable aortic neoarch or thoracoabdominally extended prosthesis of the type described below in section 2b(2)(f), entitled Expandable Structure to Accommodate Growth and Self-adjust in Size in Vascular Segment Prostheses, Lines, and Confluence Conduits Placed in Children is:
1. Less intricate and traumatic to place, increasing the pool of general surgeons, for example, who would be able to perform the operation,
2. Can be accomplished in much less time under regional anesthesia,
3. Does not require interrupting the flow of blood through the segment replaced thus eliminating an interval of anoxia and the need for cardiopulmonary bypass, and is far less likely to require replacement,
4. Less traumatic allowing application to children and adults too frail to tolerate the conventional procedure

These factors not only allow conventionally complex and risk-prone repairs to be performed by general surgeons in community hospitals but operate on end-stage children and adults unable to tolerate the conventional procedure. The capabilities imparted are not only beneficial for the very young with severe defects of the thoracic and/or thoracoabdominal aorta, for example for whom no such long-lived, dependably leak-free, expeditiously and safely placed prosthesis has been available, but for adults as well.

The durability of an expandable tie-line prosthesis as described shortly below in section 2b(2)(f), entitled Expandable Structure to Accommodate Growth and Self-adjust in Size in Vascular Segment Prostheses, Lines, and Confluence Conduits Placed in Children, is due to its:
1. Expansion in step with growth,
2. A seamless end-to-end vessel-prosthesis anastomosis structure that thwarts dehiscence,
3. Through-and-through suture, part number 232 in FIG. 37D, used to fix the outer jackets of the inline coupling jacket secured prosthesis in position,
4. The high tensile strength that precludes bursting or rupture even if directly struck, and
5. Also as shown in FIG. 37D, the incorporation of at least one drugline 8 through an accessory channel in the inlet half-jacket which allows the implanted prosthetic disorder response system to directly pipe-target an anticoagulant, antimicrobial, or any other medication to the prosthesis and the inline coupling jackets securing them at either end in response to inputs provided to the microcontroller chip, or in a hierarchically controlled system, a local endoluminal thin film clot sensor (not shown in the drawing figures).

A tie-line type prosthesis has the same structure whether to replace only the aortic arch or continue around thoracoabdominally in lieu of a conventional elephant trunk or frozen elephant truck. An aortic prosthesis, or neoarch, for example, includes supra-aortic branches for connection with inline coupling jackets to the arteries rising up into the head and brachia (arms), the number of branches, hence, inline coupling outlet jackets, depending upon whether connection on the right side is to the brachiocephalic (innominate) or directly to its separate branches.

Using the mechanism shown in FIG. 37A, as the flow of blood is switched from the native artery to the prosthesis, the relative proportion of blood seen to flow through the native aorta and its branches is reduced in the same volume as it is increased in the neoaortic arch, or prosthesis, and its branches. That is, the sum volume of blood flow is constant, never less as would result in hypoxia. This progressive transfer of blood flow from the innate vessel to the prosthesis is clearly visible through the transparent neoarch, for example, made of transparent polypropylene or amber polyetheretherketone.

These materials are suitable as little if at all susceptible to resorption. The prosthesis is additionally coated to withstand enzymatic as well as hydrolytic breakdown. Because focal repairs tend to be followed by other defects outside the original treatment area, conventional repairs allow no more than limited growth before interfering with the normal growth of tissue aside from the original defect, and because a seamless monolithic structure is most secure, expandable structure prostheses are not made to replace only a portion of a vessel such as the thoracic aorta.

Each of the prosthetic supra-aortic branches is connected to those native by inline coupling jackets positioned automatically as are the entry and exit jackets along the thoracic aorta. Accordingly, looking down on the action as depicted for a simple tie-line in FIG. 37A, there is one inlet double-jacket, one outlet double-jacket, and three more outlet double jackets each overlying its corresponding native branch. That is, the superior aspect is extended into an additional dimension. Upon completion in the transfer of flow to the prosthesis, the relevant segment of the native aorta has been replaced and any branches up to the inline coupling jackets excised.

The placement of basic outlet inline coupling jackets without the need to interrupt circulation through these vessels far less manipulative and traumatizing of diseased arch branches than is end-to-end native-to-synthetic anastomosis by stitching through application of the “trifurcated graft technique” whereby the left common carotid and subclavian are debranched and all branches rising up from the arch instead end-to-end native-to-synthetic anastomosed to the ascending arch while the patient is kept on cardiopulmonary life support would likely offer less benefit as would any other conventional repair of the aortic arch.

Different forms of the conventional technique fall short of a method that eliminates a condition of no-flow, with the possibilities for an inadequacy of oxygen delivery to the brain and kidneys, for example, and if the procedure is long in duration, the possibility for ischemia-reperfusion injury. “Despite advances in cardiac surgery and anesthesia, the rates of brain injury remain high in aortic arch surgery requiring circulatory arrest. The mechanisms of brain injury, including permanent and temporary neurologic dysfunction, are multifactorial, but intraoperative brain ischemia is likely a major contributor,” (Qu, J. Z., Kao, L. W., Smith, J. E., Kuo, A., Xue, A., and 3 others 2021. “Brain Protection in Aortic Arch Surgery: An Evolving Field,” Journal of Cardiothoracic and Vascular Anesthesia (Philadelphia, Pennsylvania) 35(4): 1176-1188; (see also, for example, Cui, Y., Liu, X., Xiong, J., Tan, Z., Du, L., and Lin, J. 2023. “Cardiopulmonary Bypass for Total Aortic Arch Replacement Surgery: A Review of Three Techniques,” Online, Frontiers in Cardiovascular Medicine (Lausanne, Switzerland) 30;10:1109401; Fang, Z., Wang, G., Liu, Q., Zhou, H., Zhou, S., and 9 others 2019. “Moderate and Deep Hypothermic Circulatory Arrest Has a Comparable Effect on Acute Kidney Injury after Total Arch Replacement with Frozen Elephant Trunk Procedure in Type A Aortic Dissection,” Online, Interactive Cardiovascular and Thoracic Surgery 29(1): 130-136; Zhou, H., Wang, G., Yang, L., Shi, S., Li, J., and 7 others 2018. “Acute Kidney Injury after Total Arch Replacement Combined with Frozen Elephant Trunk Implantation: Incidence, Risk Factors, and Outcome,” Journal of Cardiothoracic and Vascular Anesthesia (Philadelphia, Pennsylvania) 32(5): 2210-2217; LeMaire, S. A., Weldon, S. A., and Coselli, J. S. 2013. “Total Aortic Arch replacement: Current Approach Using the Trifurcated Graft Technique,” Online, Annals of Cardiothoracic Surgery (Hong Kong, China) 2(3): 347-352; Coselli, J. S. and Green, S. Y. 2009; Op cit. above in section 2a; Spielvogel, D., Etz, C. D., Silovitz, D., Lansman, S. L., and Griepp, R. B. 2007. “Aortic Arch Replacement with a Trifurcated Graft,” Annals of Thoracic Surgery,” 83(2): S791-S795, discussion S824-S831; Kazui, T. and Bashar, A. H. 2006. “Aortic Arch Replacement Using a Trifurcated Graft,” Annals of Thoracic Surgery 81(4): 1552).

Thus, replacement of the aortic arch by the means preferred offers distinct advantages over conventional surgical, endovascular, and hybrid endovascular approaches. Performed by a practiced operator and assistants, procedural time is less and the durability of the repair superior to alternative methods, the prosthesis is capable of expansion to allow growth, and the conventional means employed to arrest circulation while sustaining perfusion to the rest of the body which the present device eliminates add as many if not more risk factors than do endovascular techniques which demand a degree of manipulation within the aortic arch that can result in a midprocedural stroke (Coselli, J. S. and Green, S. Y. 2009, Op cit. above in section 2a).

Prone to proliferate beyond the focal site before or after conventional treatment such as an endoluminal stent graft, an abdominal aortic aneurysm due to a connective tissue disorder warrants replacement of much of the abdominal aorta with an expandable structure inline coupling jacket-secured tie-line type prosthesis. If not prone to propagation, an aneurysm along the abdominal aorta can be bypassed with inlet and outlet diversion jackets connecting the aorta upstream to the defect to the aorta downstream, the bypassed aneurysmal segment having been bypassed likely to atrophy eliminating a need to excise it.

If the patient has impaired aortic and carotid body function so that preconscious blood acidity and pressure sensation are lacking, a tuned shunt such as addressed below in section 2b(2)(h)(h1), entitled The ‘Tuned’ Shunt, and its subsidiary sections and shown in FIG. 22F responsive to the implanted prosthetic disorder response system controller based on its sensor inputs should be considered. Shown as tubular for use in an adult, bypass conduit, part number 196 in FIG. 22F in a child is always made with the expandable structure. In this case, the expandable structure is joined to side-entry servovalves rather than inline coupling jackets.

Due to the need to avoid encroachment on neighboring tissue by sideways extended side stems, part number 19 in the drawing figures, in crowded anatomy such as in the chest, a defect along the aortic arch such as a delimited coarctation unrelated to a connective tissue disorder that would propagate aneurysms outside the focal area of the primary lesion can be repaired with an inline aortic arch replacement conduit secured bypass connected to the arch outside the margins of the defect. Even though sidestems can be rotated to any angle and the valve adjusted in level along the aortic arch, the immediate proximity among the great vessels makes the use of side-entry devices with without the risk of irritation to neighboring tissue difficult if not impossible.

If placed in a child, the inline coupling jacket-secured bypass is made in the expandable structure form. An inline coupling jacket-secured prosthesis to replace a segment along a vessel with or without branches in an adult is the same device as the prosthesis made for placement in an infant where the operator has a fully or nearly so manually pulled open the device to its maximum dimensions. Standardized embodiments thus differ in extent rather than in separate sizes. This makes it possible to provide a standardized length of the expansion structure that allows the operator to cut it to the length needed.

However, as portions of the aorta as a whole require supraaortic branches in the chest and renal arteries in the lower abdomen, these variants must be made available as well. This complete site coverage approach supplants the skill essential to meet the demand for precision in the original case-by-case patterning and stitching of a patch, and/or the transluminal positioning of an endostent, which can injure the intima beyond the interruption in blood flow required for both.

Eliminating a period of no blood flow, hence, anoxia, and general anesthesia, a tie-line vascular prosthesis secured at either end by inline coupling jackets eliminates much of the mid- and postprocedural factors that result in complications and eventuate in the premature need for revision. Unlike an endograft, bypasses with ductus side-entry jackets, vascular servovalves, or inline coupling jackets at either end such as employed in conduits for use along the thoracic aorta are not susceptible to endoleak.

This one factor in addition to the advantages previously specified eliminates numerous adverse sequelae associated with the conventional placement of an edge-stitched patch or transluminally positioned stent-graft to effect such a repair. Precluding the need for reoperation eliminates the potential for most adverse consequences of chest surgery, however, the need to gain entry, hence, unless a relatively minor procedure accomplished transluminally, the pain of a thoracic incision cannot be avoided.

Another disadvantage of edge-stitched patches as in a patch aortoplasty and other types of vascular repair is that the patch interferes with growth at the patch and stunts the growth around it (references cited above in this section). Because many defects of the thoracic aorta impede normal circulation at birth, repair tends to take place then—at precisely the worst time-when postnatal growth commences. Moreover, the trauma of conventional repair considerable, neonates often affected by more than one congenital defect are in a condition unprepared to withstand the surgery which is deferred. Anoxia and cardiopulmonary bypass support-free, intracorporeally seamless and leak-proof repair of aneurysms and coarctations of the aortic arch under regional or local anesthesia with targetable drug delivery eliminates numerous complications encountered peri- and postnatally with conventional repairs.

That to replace an abdominal aortic aneurysm with an inline coupling jacket-secured prosthesis or bypass it with a side-entry jacket- or valve-secured prosthesis, neither of which require interruption in blood flow, offers advantages over the use of a conventional endovascular stent-graft which is susceptible to leakage, migration, and intimal injury during transluminal placement, should be evident, and the same may be said for the repair of an aneurysm or coarctation of the aortic arch.

In the abdomen, an infrarenal neck inadequate for the placement of an endograft can be negotiated by connection of the bypass either to the aorta or to the iliacs (Diehm, N., Katzen, B. T., Samuels, S., Pena, C., Powell, A., and Dick, F. 2008. “Sixty-four-detector CT Angiography of Infrarenal Aortic Neck Length and Angulation: Prospective Analysis of Interobserver Variability,” Journal of Vascular and Interventional Radiology (Reston, Virginai) 19(9): 1283-1288; Leurs, L. J., Kievit, J., Dagnelie, P. C., Nelemans, P. J., and Buth, J. 2006. “Influence of Infrarenal Neck Length on Outcome of Endovascular Abdominal Aortic Aneurysm Repair,” Journal of Endovascular Therapy (Phoenix, Arizona) 13(5): 640-488).

The advantages of inline coupling jacket-secured expansion structured tie-line type vascular segment prostheses in avoiding an interruption in blood flow, seamless placement that precludes dehiscence and migration, or the need for regional or local anesthesia must compete with the advantages of an endograft in eliminating the need for open surgery at the risk of long-term complications. Unlike an expansion structured prosthesis, placed in a child, an endovascular stent graft has a finite life before its repair or replacement will become necessary at which time the reoperation may require open surgery.

Open repair of endovascular stent grafts is common (see, for example, Geraedts, A. C. M., Mulay, S., van Dieren, S., Koelemay, M. J. W., and Balm, R.; 2021. “Analysis of Outcomes after Endovascular Abdominal Aortic Aneurysm Repair in Patients with Abnormal Findings on the First Postoperative Computed Tomography Angiography,” Online, Journal of Endovascular Therapy (Thousand Oaks, California) 28(6): 878-887; Blackstock, C. D. and Jackson, B. M. 2020. “Open Surgical Repair of Abdominal Aortic Aneurysms Maintains a Pivotal Role in the Endovascular Era,” Online, Seminars in Interventional Radiology (New York, New York) 37(4): 346-355. Perini, P., de Troia, A., Tecchio, T., Azzarone, M., Bianchini Massoni, C., Salcuni, P., Freyrie, A. 2017. “Infrarenal Endograft Clamping in Late Open Conversions after Endovascular Abdominal Aneurysm Repair,” Online, Journal of Vascular Surgery (New York, New York) 66(4): 1048-1055; Brinster, C. J., Fairman, R. M., Woo, E. Y., Wang, G. J., Carpenter, J. P., and Jackson, B. M. 2011. “Late Open Conversion and Explantation of Abdominal Aortic Stent Grafts,” Online, Journal of Vascular Surgery (New York, New York) 54(1): 42-46; Klonaris, C., Lioudaki, S., Katsargyris, A., Psathas, E., Kouvelos, G., and 3 others 2014. “Late Open Conversion after Failed Endovascular Aortic Aneurysm Repair,” Online, Journal of Vascular Surgery (New York, New York) 59(2): 291-297; Nabi, D., Murphy, E. H., Pak, J., and Zarins, C. K. 2009. “Open Surgical Repair after Failed Endovascular Aneurysm Repair: Is Endograft Removal Necessary? Online, Journal of Vascular Surgery (New York, New York) 50(4): 714-721). Provided a younger patient has the stamina for the open procedure to place a likely lifelong prosthesis, the endovascular placement of a stent graft that may necessitate an open procedure to repair or replace it anyway should be considered a halfway measure.

Partial Diversion of Flow Combined with Directly Pipe-Targeted Medication as Means for Reducing Local Hypertension

To protect a hypertensive vessel such as a pulmonary artery or the portal vein and the tissue or organ it perfuses, a serious elevation in blood pressure due to obstruction posed by the downstream organ supplied must be abated before the disease progresses. Untreated these conditions lead to death (see, for example, Kibrit, J., Khan, R., Jung, B. H., and Koppe, S. 2018. “Clinical Assessment and Management of Portal Hypertension,” Online, Seminars in Interventional Radiology (New York, New York) 35(3): 153-159; Lan, N. S. H., Massam, B. D., Kulkarni, S. S., and Lang, C. C. 2018. “Pulmonary Arterial Hypertension: Pathophysiology and Treatment,” Online, MDPI [Multidisciplinary Digital Publishing Institute] Diseases (Basel, Switzerland 16;6(2): 38).

Two means for ameliorating such a condition are to divert a portion of the blood to another larger vessel and directly targeting antihypertensives to the affected vessel. Such is not meant to supplant the concurrent use of oral medication (see, for example, Khalil, H. and Zeltser, R. 2023. “Antihypertensive Medications,” Online, Treasure Island, Florida: StatPearls Publishing Co.). Simple side-entry jackets can implement diversion through a one-way shunt passively, while vascular servovalves can implement a controllable, or meterable, here referred to a ‘tuned’ shunt addressed below in section 2b(2)(h)(h1), entitled The ‘Tuned’ Shunt, its subsidiary sections, and shown in FIG. 22F. Such a shunt is used continuously or intermittently to adjust the volume of blood tapped off for delivery into another larger vessel in response to the feedback received by the implanted master control microprocessor from one or more sensors along the affected vessel.

A servovalve can continuously adjust and preserve the pressure upstream to it by adjusting the fraction of blood to be diverted. The valve closes to increase, and opens to reduce, the upstream pressure. Closing also increases the pressure of the tapped off effluent eliminating much if not all of the excessive pressure in the affected vessel. The extravasatant tapped off thus passes into an outlet catheter or shunt for delivery into a larger of like kind as to artery or vein.

The need to prevent high blood pressure, often associated with as the cause of injury to downstream tissue or an aneurysm, can be accomplished by a tuned shunt as addressed below in section 2b(2)(f), entitled Expandable Structure to Accommodate Growth and Self-adjust in Size in Vascular Segment Prostheses, Lines, and Confluence Conduits Placed in Children, and section 2b(2)(h), entitled Vascular Pressure Relief, and its subsections. In preventing injury to downstream tissue as long as it takes to alleviate the cause for the elevated pressure, the tapping of an amount of blood no greater than necessary to lower the pressure to normotensive is not curative but rather palliative; alleviating the cause may call for treatment ranging from medical to tissue or organ transplantation.

Both the conduit diversion jackets and associated one-way passive or controlled pressure relief valve are provided with accessory channels which allow the direct pipeline targeting of antihypertensive medication such as nitrates locally, or topically, into the aortic arch. The coordinated release into the arterial tree of systemic antihypertensives by the implanted control microprocessor can be through accessory channel-inserted druglines optimally positioned elsewhere in the body, such as along the renal arteries or the portal vein.

A side-entered synthetic bypass can provide stitchless and leakless connection to the substrate stumps while eliminating the need for cardiopulmonary bypass or hypothermic circulatory arrest, for example (Cramer, J. W., Ginde, S., Bartz, P. J., Tweddell, J. S., Litwin, S. B., and Earing, M. G. 2013, Op cit., above in section 2a, entitled Background of the Invention. Geneal; Roth, M., Lemke, P., Schonburg, M., Klovekom, W. P., and Bauer, E. P. 2002. “Aneurysm Formation after Patch Aortoplasty Repair (Vossschulte): Reoperation in Adults with and without Hypothermic Circulatory Arrest,” Annals of Thoracic Surgery (Amsterdam, North Holland) 74(6): 2047-2050).

The means used in conventional repair can in themselves preclude patients in need of arch reconstruction from repair, or at the least, jeopardize their outcomes (“Open total arch replacement is a robust operation that can be performed with excellent results. However, it requires cardiopulmonary bypass and the interruption in circulation through the affected segment, and therefore, may not be tolerated by all patients,” (Faulds, J., Sandhu, H. K., Estrera, A. L., and Safi, H. J. 2016. “Minimally Invasive Techniques for Total Aortic Arch Reconstruction,” Methodist DeBakey Cardiovase Journal 12(1): 41-44). Accordingly, the elimination of cardiopulmonary bypass, ischemia, and general anesthesia expands the pool of patients able to undergo a total open arch replacement.

The diversity of abdominal and aortic arch defects at different stages in life from infancy to adulthood inclusive of hypoplasty, coarctation, and aneurysm, whether the repair is initial or a revision makes generalizations as to treatment difficult and reduction to one basic approach ill-advised. Here there is provided an alternative to conventional measures which should prove superior. Bypass with the aid of side-entry or vascular servovalve diversion jackets is intended to provide an alternative when conventional treatment—the placement of an endovascular stent-graft, excision with end-to-end anastomosis, excision with the placement of a Dacron® patch, with the need for cardiopulmonary bypass and general anesthesia would predispose to complications adscititious relative to the substantive surgical procedure.

End-to-side anastomosis which detaches the descending aorta for attachment beneath the innominate and left carotid leaves a blind end or ‘cul de sac’ downstream of the left subclavian artery, which to avoid the accumulation of clot and possible obstruction of the left subclavian, should be targeted with an anticoagulant (see, for example, Dharmapuram, A. K., Ramadoss, N., Verma, S., Vejendla, G., and Ivatury, R. M. 2018. “Early Outcomes of Modification of End to Side repair of Coarctation of Aorta with Arch Hypoplasia in Neonates and Infants,” Annals of Pediatric Cardiology (Mumbai [Bombay] India) 11(3): 267-274, FIG. 4).

Configurations that provide for a more constant radius of curvature and continuity of the thoracic aorta with no abrupt cutoff past the point of redirection had been shown earlier (see, for example, Lee, M., d'Udekem, Y. and Brizard, C. 2013. “Coarctation of the Aorta,” in Da Cruz, E., Ivy, D., and Jaggers, J. (eds), Pediatric and Congenital Cardiology, Cardiac Surgery and Intensive Care, London, England: Springer, pages 1631-1646)(see also, Donazzan, L., Crepaz, R., Stuefer, J., and Stellin, G. 2014. “Abnormalities of Aortic Arch Shape, Central Aortic Flow dynamics, and distensibility predispose to hypertension after Successful Repair of Aortic Coarctation,” World Journal for Pediatric and Congenital Heart Surgery 5(4): 546-553). Early and midterm follow-up reports pertaining to repairs done in neonates and infants may prove misleading for these patients as adults.

In a patient without a congenital tissue disorder so that aneurysms might develop anywhere along the aorta, an aneurysm susceptible to imminent rupture should be surgically repaired or bypassed. In a patient with a congenital tissue disorder, more extensive coverage is essential. Generally, vascular defects to include a coarctation, aneurysm, or hypoplastic segment that result in the inadequate oxygenation, or chronic hypoxia, and often hypertension, of their respective supply territories and pose the risk of structural failure should be repaired or bypassed upon detection.

Unless the defect can be repaired so that adverse sequelae have been precluded with confidence, a heart or thoracic aorta should be replaced, the heart through transplantation, the aorta through replacement with a prosthesis. The prompt replacement of a heart too malformed to be repaired as to instate normal circulation and the maldevelopment that results in all organs and tissues due to nonpulsatile flow and chronic hypoxia are addressed in sections 2a and 2a(1), entitled Congenital Defects of the Heart and Other Organs—The Importance of Prompt Intervention.

Whether the lesion is primary or de novo or consequent to the earlier repair of the lesion, side-entry jackets and valves make possible a bypass for a coarctation, interruption, aneurysm, or hypoplastic segment along a larger vessel containing the defect. For example, a coarctation of the aorta in an otherwise normal neonate or infant which to repair conventionally would necessitate excising too much of the aorta in order to end-to-end anastomose can be repaired thus. Another factor that prompts the removal and inclusion in a prosthesis of much tissue up- and downstream to the defect is that the weakened condition of the wall surrounding a coarctation, for example, makes the tissue prone to aneurysm and the leaking if not separation of sutures.

Because a patch aortoplasty to bridge the gap cannot expand with growth up to adolescence much less adulthood, such a repair consigns the patient to painful and complication-prone later revision. By contrast, a bypass made with the expanding structure addressed below in section 2b(2)(f), entitled Expandable Structure to Accommodate Growth and Self-adjust in Size in Vascular Segment Prostheses, Lines, and Confluence Conduits Placed in Children, can remain as an effective repair for years, perhaps, even to the end of life. A bypass cannot be entered into upstream to the supraaortic branches as this would bypass blood past these.

For this reason, if the defective condition of the thoracic aorta is too extensive to allow a limited bypass of the defect, a seamless, leak-free, and migration-proof replacement thoracic aorta somewhat more extensive than that depicted in FIG. 36F is placed as described above in section 2b(2)(c), entitled Vascular Segment Prostheses: Inline Coupling Jackets and Bridging, or Tie-lines, and its subordinate sections.

Ductus side-entry jackets and valves used as inflow and outflow connectors for a vascular bypass and intravascular diversion servovalves which to reduce the pressure through the input vessel and bypass by diverting a volume of blood controlled by the implanted microcontroller into another larger vessel make possible a gradual redirection of blood. Redirection can be by a diversion chute with no sharp bend from the inlet to the outlet and with no part that protrudes into the lumina. Fistulas are generally excised with the origins, or points of connection at either end, stitched closed if small, or patched if larger.

Whether along the thoracic aorta, adequate durability in the original repair of a defect can avert the need for revision. Vascular specialists have long sought improved durability (see, for example, Gropler, M. R. F., Marino, B. S., Carr, M. R., Russell, W. W., Gu. H., and 3 others 2019. “Long-term Outcomes of Coarctation Repair through Left Thoracotomy,” Annals of Thoracic Surgery (Amsterdam, North Holland, Netherlands) 107(1): 157-164; Price, J. D. and LaPar, D. J. 2019. “The Challenges of Redo Aortic Coarctation Repair in Adults,” Current Cardiology Reports 21(9): 99; Tsang. V., Haapanen, H., and Neijenhuis, R. 2019. “Aortic Coarctation/Arch Hypoplasia Repair: How Small is Too Small,” Seminars in Thoracic and Cardiovascular Surgery. Pediatric Cardiac Surgery Anual 22:10-13; Bai, J., Liu, Y., Jin, J., Li, J., Ji. X., and Qu. L. 2018. “Single-stage Endovascular Management of Complicated Thoracic Aorta Coarctation Concurrent with Aortic Arch Aneurysm Using a Novel Fenestration Device,” Journal of Thoracic Disease (Hong Kong, China) 10(4): 2474-2480; Beckmann, E. and Jassar, A. S. 2018. “Coarctation Repair-redo Challenges in the Adults: What To Do?,” Journal of Visualized Surgery 4:76; Cui, H. J., Chen, J. M., Zhuang, J., Cen, J. Z., Xu, G., and Wen, S. S. 2018. “Short and Midterm Results of Surgical Treatment of Interrupted Aortic Arch,” (in Chinese with English abstract at Pubmed), Zhonghua Wai Ke Za Zhi [Chinese Journal of Surgery] (Beijing, China) 56(12): 916-921; Dharmapuram, A. K., Ramadoss, N., Verma, S., Vejendla, G., and Ivatury, R. M. 2018, Op cit. above in this section; Poncelet, A. J., Henkens, A., Sluysmans, T., Moniotte, S., de Beco, G., and 3 others 2018. “Distal Aortic Arch Hypoplasia and Coarctation Repair: A Tailored Enlargement Technique,” World Journal for Pediatric and Congenital Heart Surgery 9(5): 496-503; Zhu, J., Zhao, L., Dai, X., Luo, Y., Fan, H., and 3 others 2018. “Fenestrated Thoracic Endovascular Aortic Repair Using Physician Modified Stent-grafts for Acute Type B Aortic Dissection with Unfavourable Landing Zone,” European Journal of Vascular and Endovascular Surgery (London, England) 55(2): 170-176; Zou, M. H., Ma. L., Xia, Y. S., Yang, S. C., Chen, W. D., Cao, F., and Chen, X. X. 2018. “End-to-side Anastomosis for Interrupted Aortic Arch in Neonates and Infants),” (in Chinese with English abstract available at Pubmed), Zhonghua Wai Ke Za Zhi [Chinese Journal of Surgery] (Beijing, China) 56(3): 217-220; Lu, W. H., Fan, C. S., Chaturvedi. R., Lee, K. J., Manlhiot. C., and Benson, L. 2017. “Clinical Impact of Stent Implantation for Coarctation of the Aorta with Associated Hypoplasia of the Transverse Aortic Arch,” Pediatric Cardiology 38(5): 1016-1023; Qin, J., Zhao, Z., Wang. R., Ye. K., Li, W., and 13 others 2017. “In Situ Laser Fenestration is a Feasible Method for Revascularization of Aortic Arch during Thoracic Endovascular Aortic Repair,” Journal of the American Heart Association (Oxford, England) 6(4). pii: e004542; Wang. T., Shu, C., Li, M., Li, Q. M., Li, X., and 4 others 2017. “Thoracic Endovascular Aortic Repair with Single/Double Chimney Technique for Aortic Arch Pathologies,” Journal of Endovascular Therapy 24(3): 383-393; Quennelle, S., Powell, A. J., Geva. T., and Prakash, A. 2015. “Persistent Aortic Arch Hypoplasia after Coarctation Treatment is Associated with Late Systemic Hypertension,” Journal of the American Heart Association (Oxford, England) 4(7). pii: e001978; Liu, J. Y., Jones, B., Cheung, M. M., Galati, J. C., Koleff, J., and 4 others 2014. “Favourable Anatomy after end-to-side Repair of Interrupted Aortic Arch,” Heart, Lung, and Circulation 23(3): 256-264; Lee, M., d'Udekem, Y. and Brizard, C. 2013, Op cit. above in this section; Rakhra, S. S., Lee, M., Iyengar, A. J., Wheaton, G. R., Grigg. L., and 3 others 2013. “Poor Outcomes after Surgery for Coarctation Repair with Hypoplastic Arch Warrants More Extensive Initial Surgery and Close Long-term Follow-up,” Interactive Cardiovascular and Thoracic Surgery (Amsterdam, North Holland, Netherlands) 16(1): 31-36; additional references cited below in this section; Allen, B. S., Haldorsson, A. O. Barth, M. J., and Ibawi, M. N. 2000, Op cit., above in section 2a, entitled Background. General).

Eliminating the need for cardiopulmonary bypass and general anesthesia, use of ductus side-entry jackets, valve-jackets, and servovalve-jackets to bypass a coarctation or aneurysm is not only safer to accomplish but is at least as, and probably more, dependable as critically more accommodating of growth than anastomosis, patching, or stent-grafting. Moreover, the side-entry jacket or valve accessory channels allow the direct release into the bypass of an anticoagulant, antimicrobial, or any other drug. Synthetics unresponsive to growth hormone, to allow for growth in a neonate or infant, the small bypass inflow and outflow jackets have foam linings made proportionally thicker than those for use in an adult to accommodate the pulse as well as growth. In the gastrointestinal tract, peristalsis is unrestrained.

To accommodate growth and comply with the pulse, side-entry jackets, valves, and inline coupling jackets incorporate a thick parylene coated viscoelastic polyurethane foam lining. Made as delineated below in section 2b(2)(f), entitled Expandable Structure to Accommodate Growth and Self-adjust in Size in Vascular Segment Prostheses, Lines, and Confluence Conduits Placed in Children, vascular segment prostheses such as a replacement aortic arch, or neoarch, provides these properties without a special lining.

With such a prosthesis, the spontaneous increase in dimensions of the aortic arch in step with growth plus the seamless structure preclude dehiscence and eliminate the need for a reoperation, so that a repair thus should into adulthood if not for life. This in stark contrast to the maldevelopment of the thoracic aorta as a whole following conventional patch or endoluminal repair. “Infants with coarctation of the aorta frequently require a corrective operation during the first year of life. These patients frequently have a smaller than normal transverse aortic arch. Despite good repairs with different techniques, the proximal transverse aortic arch often remains smaller than normal,” (Myers, J. L., McConnell. B. A., and Waldhausen, J. A. 1992. “Coarctation of the Aorta in Infants: Does the Aortic Arch Grow after Repair?,” Online, Annals of Thoracic Surgery (Amsterdam, North Holland, Netherlands) 54(5): 869-875; see also Eldadah, O. M., Alsalmi, A. A., Diraneyya, O. M., Hrfi, A. A., Mohammed, M. H. A., Valls, M. L., and Alghamdi, A. A. 2023. “Progressive Changes in Residual Gradient after Aortic Coarctation Rrepair and Its Role in the Prediction of Reintervention: A Longitudinal Data Analysis,” Annals of Pediatric Cardiology (Mumbai [Bombay] India) 16(3): 182-188; Panzer, J., Bove, T., Vandekerckhove, K., and De Wolf, D. 2022. “Hypertension after Coarctation Repair—A Systematic Review,” Online, Translational Pediatrics (Hong Kong, China) 11(2): 270-279; Dijkema, E. J., Dik, L., Breur, J. M. P., Sieswerda, G. T., Haas, F., Slieker, M. G., and Schoof, P. H. 2021. “Two Decades of Aortic Coarctation Treatment in Children; Evaluating Techniques,” Online, Netherlands Heart Journal (Leusden, Netherlands) 29(2): 98-104; Sojak, V. and Hazekamp, M. 2021. “Paradigm Shift in Treatment of Coarctation of the Aorta? Online, Netherlands Heart Journal (Leusden, Netherlands) 29(2): 69-70; Lu, W. H., Fan, C. S., Chaturvedi, R., Lee, K. J., Manlhiot, C., and Benson, L. 2017. “Clinical Impact of Stent Implantation for Coarctation of the Aorta with Associated Hypoplasia of the Transverse Aortic Arch,” Pediatric Cardiology (New York, New York) 38(5): 1016-1023; Rakhra, S. S., Lee, M., Iyengar, A. J., Wheaton, G. R., Grigg, L., and 3 others 2013. “Poor Outcomes after Surgery for Coarctation Repair with Hypoplastic Arch Warrants More Extensive Initial Surgery and Close Long-term Follow-up,” Online, Interactive Cardiovascular and Thoracic Surgery 16(1): 31-36; Weber, H. S., Cyran, S. E., Grzeszczak, M., Myers, J. L., Gleason, M. M., and Baylen, B. G. 1993. “Discrepancies in Aortic Growth Explain Aortic Arch Gradients during Exercise,” Online, Journal of the American College of Cardiology (New York, New York) 21(4): 1002-1007; Siewers, R. D., Ettedgui, J., Pahl, E., Tallman, T., and del Nido, P. J. 1991. “Coarctation and Hypoplasia of the Aortic Arch: Will the Arch Grow?, Online, Annals of Thoracic Surgery (Amsterdam, North Holland, Netherlands) 52(3): 608-614).

Averting the need for a painful revision eliminates not only the malformity to ensue following a conventional procedure but also the maldevelopment of the child attendant thereupon, the serious and irreversible maldevelopment secondary to defective circulation affecting the entire body to include the brain, adversely affecting intellectual ability for life. Another advantage in the use of the expandable neoarch or another prosthesis of like structure which expands with growth is the automatic monitoring and maintenance afforded by the sensors and direct drug delivery of the druglines of the implanted ambulatory and automatic disorder response system.

All side-entry jackets, connectors, valves, confluence conduits, and prostheses incorporate the sensors and at least one accessory channel for the insertion of a drugline, part number 8 in the drawing figures, to allow the implanted disorder control system to immediately detect the need for, identify, and directly pipe-target medication into the device and the vessel or vessels to which it is attached. Provided it is small and delimited, if possible, conventional repair of a defect such as a coarctation or interruption is best accomplished by excision and direct end-to-end anastomosis of the stumps with no patch, stent-graft, or flap to restrict growth.

Not only do these prevent normal growth of the thoracic aorta, but they lack the monitoring to instantly detect the need for, and directly target medication to the site as specified in the prescription-program of an implanted automatic disorder response system. None have any of the critical advantages of the replacement thoracic aorta described herein. In a small child, larger defects conventionally repaired through the placement of a woven polyethylene terephthalate (Dacron®, E.I. DuPont de Nemours, Incorporated, Wilmington, Delaware) patch sutured to the cut ends of the aortic arch usually has a finite life, after which a child will have to undergo one or more painful revision operations.

The treatment of vascular tissue susceptible to structural failure includes the treatment of endothelial dysfunction and of collagen congenitally defective as to lack adequate strength. Automatically administered by an implanted disorder control system, drugs for release from implanted drug reservoirs into the systemic and pulmonary circulation must be in liquid form. “Experimental and clinical studies have demonstrated that a variety of currently used and investigational drugs, such as angiotensin-converting enzyme inhibitors, angiotensin ATI receptors blockers, angiotensin-(1-7) [but see, Faugeroux et al. 2013 below as regards vascular, or Type IV rather than hypermobile Ehlers-Danlos syndrome, which is due to a congenital insufficiency of Type III collagen], antioxidants, beta-blockers, calcium channel blockers, endothelial NO synthase enhancers, phosphodiesterase 5 inhibitors, sphingosine-1-phosphate and statins, exert endothelial protective effects,” (Su, J. B. 2015. “Vascular Endothelial Dysfunction and Pharmacological Treatment,” Online, World Journal of Cardiology 7(11): 719-741)(see also Miklovic, T. and Sieg, V. C.2023. “Ehlers-Danlos Syndrome,” Online, Treasure Island, Florida: StatPearls Publishing Co.; Legrand, A., Guery, C., Faugeroux, J., Fontaine, E., Beugnon, C., and 7 others 2022. “Comparative Therapeutic Strategies for Preventing Aortic Rupture in a Mouse Model of Vascular Ehlers-Danlos Syndrome,” Online, PLoS [Public Library of Science] Genetics (San Francisco, California) 18(3): e1010059; Melsom, H. S., Heiestad, C. M., Eftestol, E., Torp, M. K., Gundersen, K., and 4 others 2022. “Reduced Arterial Elasticity after Anabolic-Androgenic Steroid Use in Young Adult Males and Mice,” Online, Scientific Reports (London, England) 12(1): 9707; Evans, C. E., Iruela-Arispe, M. L., and Zhao, Y. Y. 2021. “Mechanisms of Endothelial Regeneration and Vascular Repair and Their Application to Regenerative Medicine,” Online, American Journal of Pathology (Philadelphia, Pennsylvania) 191(1): 52-65; Dubacher N, Munger J, Gorosabel M C, Crabb J, Ksiazek A. A, and 9 others 2020. “Celiprolol but Not Losartan Improves the Biomechanical Integrity of the Aorta in a Mouse Model of Vascular Ehlers-Danlos Syndrome,” Online, Cardiovascular Research [British Medical Association] (London, England) 116(2): 457-465; Faugeroux, J., Nematalla, H., Li, W., Clement, M., Robidel, E., and 10 others 2013. “Angiotensin II Promotes Thoracic Aortic Dissections and Ruptures in Col3a1 Haploinsufficient Mice,” Online, Hypertension (Dallas, Texas) 2013 62(1): 203-208; Ehlers-Danlos syndrome is also addressed above in section 2a, entitled Background of the Invention. General).

Basic, or fixed, side-entry diversion jackets molded in a single piece with a fixed diversion chute provide for the nonadjustable rate and pressure of flow through bypass inflow and outflow jackets. Dynamic control over the volume of blood tapped, or bled, off from a hypertensive vessel for diversion into another larger vessel for the purpose of adjusting the pressure through the vessel to the normotensive in accordance with the activity level of the subject, for example, requires servovalves. The controlled pressure relief afforded by an implanted disorder response system is addressed below in section 2b(2)(h)(h1), entitled The ‘Tuned’ Shunt, and its subsidiary sections, and shown in FIG. 22F which will usually apply not onto to the portal vein but the pulmonary as well.

With vascular servovalves, an implanted microcontroller can continuously or intermittently use the input from a thin film pressure sensor along the endothelium within the affected vessel to dynamically adjust the volume, hence, the pressure, of the blood, flowing through the affected vessel. Application thus to prevent injury to tissue up- and/or downstream to the hypertensive vessel is not a cure but meant as an interim expedient to normalize the pressure until the medical cause for the hypertension is diagnosed and counteracted.

However, a master control microprocessor administering a hierarchical control system can be extended to incorporate sensors not only for legitimate, that is, essentially separate disease, but that monitor etiological and numerous sequelary symptoms of a serious primary disease such as hyperthyroidism or diabetes for which its prescription-program specifies the responsive action. This still does not constitute, but can effectively simulate, a cure.

However, as explained below in section 2b(2)(h)(h1), entitled The ‘Tuned’ Shunt, and its subsidiary sections, while devices such as those depicted in drawing FIGS. 17B and 22F in the context of monomorbid disease can be controlled by a relatively simple and suitably encapsulated implanted microcontroller chip in a more or less isolated manner, the wider context of etiological events and secondary symptoms in mono- or multimodal disease can also be assigned to sensors in a hierarchically organized control system administered by a master control microprocessor executing a prescription-program which does indeed extend to such associated symptoms.

Extended thus, the consequence is still not a cure as such but can nevertheless be highly effective in suppressing the disease to effectively approximate a cure. In the treatment of multiple interrelated comorbidities, the number of sensors is limited to those indicative of events that represent a medical emergency and warrant telemetric reporting to the clinic. The best system is that simplest but adequate. To reduce the system burden in terms of the number of drugs to be controlled, only those which affect the most important health-related symptoms are included. In patients trusted to adhere to the prescription regimen, drugs which rarely pose a problem when a proper dose is dispersed throughout the circulation and tend to be self-targeting such as loperamide hydrochloride or phenazopyridine are reserved for the oral route. That pipe-targeted drugs must be topically effective may necessitate that these be formulated in post-liver pass form.

To emulate bypass through spontaneously formed collaterals as the remedy of nature-which compared to surgery and angioplasty is relatively safe and free of complications—an endoscopically placed bypass as a synthetic collateral or collaterals, can be placed at birth, or if detected in utero, prenatally or as soon thereafter as complications appear, thus averting the maldevelopment that would affect the rest of the body, much less lower body neurological damage, over the time natural collaterals would take to develop. In a very young patient, a pulse and growth-compliant connecting tube as described above in section 2a can be used. To prevent its breakdown, the viscoelastic polyurethane foam lining the jacket is encapsulated within an outer layer of parylene or gold of pliancy approximating that of the substrate foam.

Inline coupling jackets securing either end of an expandable structure formed tie-line type prosthesis through which the flow of blood need not be affected, and side-entry servovalves securing either end where flow is to be controlled, that compliant foam lines these means not only that placed in an infant the line will allow for growth over a period of years. With servovalves, the line will make possible the automatic adjustment in blood pressure responsive to rigorous exertion. To normalize oxygenation and reduce aortic and left heart hypertension, a coarctation of the aorta is conventionally repaired transluminally, with or without stenting, stent-grafting, or surgically with a patch.

The approach here is to preempt the recurrence of a defect along the thoracic aorta-often predictable in a neonate or infant—by assuming that its tissue is defective as genetically predisposed to structural breakdown by replacing it in toto with a seamless prosthesis capable of growing in step with a neonate or infant, which is too great in tensile strength to succumb to the pressure anywhere along the aorta, and if appropriate directly targetable with medication through one or more druglines leading to the inlet inline coupling jacket securing it upstream or to the prosthesis itself.

More specifically, if hypertension is present, the prosthesis can be directly targeted for the release of antihypertensives through one or more druglines leading to the inlet inline coupling jacket securing it upstream such as part number 8 in FIGS. 36A and 36F or to the prosthesis itself. While the direct targeting of medication to the site should afford a significant measure of relief, an additional measure available to dispel hypertension is connection to a tuned shunt such as addressed below in section 2b(2)(h)(h1), entitled The ‘Tuned’ Shunt, and its subsidiary sections and FIG. 22F. The shunt taps off a volume of blood to lower the aortic pressure to the normotensive. With a great vessel such as the aorta, diversion thus is to more than one smaller vessel.

Conventional methods of repair—even when redone to repair an earlier repair—can pose serious complications, to include aneurysm formation and recoarctation (see, for example, Zhao, Z., Pan, Z., Wu. C., Tian, J., Qin. J., Zhang. Y., and Jin. X. 2023. “Risk Factors for Recurrence after Surgical Repair of Coarctation of the Aorta in Children: A Single-center Experience Based on 51 Children,” Online, Frontiers in Cardiovascular Medicine (Lausanne, Switzerland) 10:1144755; Dias, M. Q., Barros, A., Leite-Moreira, A., and Miranda, J. O. 2020. “Risk Factors for Recoarctation and Mortality in Infants Submitted to Aortic Coarctation Repair: A Systematic Review,” Pediatric Cardiology (New York, New York) 41(3): 561-575; Lehnert. A., Villemain, O., Gaudin, R., Meot, M., Raisky, O., and Bonnet, D. 2019. “Risk Factors of Mortality and Recoarctation after Coarctation Repair in Infancy,” Online, Interactive Cardiovascular and Thoracic Surgery (Oxford, England) 29(3): 469-475; Adamson, G., Karamlou T., Moore, P., Natal-Hernandez, L., Tabbutt, S., and Peyvandi, S. 2017. “Coaretation Index Predicts Recurrent Aortic Arch Obstruction following Surgical Repair of Coarctation of the Aorta in Infants,” Pediatric Cardiology (New York, New York) 38(6): 1241-1246; Truong, D. T., Tani, L. Y., Minich, L. L., Burch, P. T., Bardsley, T. R., and Menon, S. C. 2014. “Factors Associated with Recoarctation after Surgical Repair of Coarctation of the Aorta by Way of Thoracotomy in Young Infants,” Pediatric Cardiology (New York, New York) 35(1): 164-170).

Following correction by balloon angioplasty, fully 80 percent of infants experience recoarctation, stents in infants are outgrown, 6 percent of stent-graft-corrected coarctations develop aneurysms, some are left with hypertension, and some with spinal cord ischemia (see, for example. Beckmann, E. and Jassar, A. S. 2018, Op cit.; Parsa, P., Eidt. J., Rios, A., Gable, D., and Vasquez, J. Jr. 2018. “Case Report: An Innovative Endovascular Technique for Repair of Descending Thoracic Aortic Aneurysm following an Open Coarctation Repair,” Annals of Vascular Surgery 46:205.e1-205.e4; Baumgarten, H., Squiers, J. J., Brinkman, W. T., and Vasquez, J. Jr. 2017. “Endovascular Technique for Repair of Descending Thoracic Aortic Aneurysm after Coarctation Operation,” Annals of Thoracic Surgery 103(2): e167-e169; Oliveira, N. F., Castro, J. A., Martins, J. D., Quintas, A., Laranjo, S., and 3 others 2016. “Bridging Thoracic Endovascular Aneurysm Repair for a Late Rupture following Aortic Coarctation Angioplasty,” Cardiology in the Young (Cambridge, England) 26(5): 957-960; Joseph, G., Premkumar, P., Thomson, V., Varghese, M., Selvaraj. D., and Sahajanandan, R. 2016. “Externalized Guidewires to Facilitate Fenestrated Endograft Deployment in the Aortic Arch,” Journal of Endovascular Therapy 23(1): 160-171; Suradi, H. and Hijazi, Z. M. 2015. “Current Management of Coarctation of the Aorta,” Global Cardiology Science and Practice 2015(4): 44; Chiu, H. H., Wang, J. K., Chen, Y. S., Chiu, I. S., Chang, C. I., and 5 others 2013. “Long-term Outcomes of Native Coarctation of the Aorta after Balloon Angioplasty or Surgical Aortoplasty in Newboms and Young Infants Less Than 3 Months of Age,” Acta Cardiologica Sinica 29(2): 168-174; Vergales, J. E., Gangemi, J. J., Rhueban, K. S., and Lim, D. S. 2013. “Coarctation of the Aorta—The Current State of Surgical and Transcatheter Therapies,” Current Cardiology Reviews 9(3): 211-219; Cramer, J. W., Ginde, S., Bartz, P. J., Tweddell, J. S., Litwin, S. B., and Earing, M. G. 2013. Op cit. above in section 2a; Juszkat. R., Perek, B., Zabicki, B., Trojnarska, O., Jemielity, M., and 3 others 2013. “Endovascular Treatment of Late Thoracic Aortic Aneurysms after Surgical Repair of Congenital Aortic Coarctation in Childhood,” Online, PLoS [Public Library of Science] 8(12): e83601; Botta, L., Russo, V., Oppido, G., Rosati, M., Massi, F., and 3 others 2009. “Role of Endovascular Repair in the Management of Late Pseudo-aneurysms following Open Surgery for Aortic Coarctation,” European Journal of Cardiothoracic Surgery 36(4): 670-674; Karamlou. T/, Bernasconi. A., Jaeggi. E., Alhabshan. F., Williams. W. G., and 3 others 2009. “Factors Associated with Arch Reintervention and Growth of the Aortic Arch after Coarctation Repair in Neonates Weighing Less than 2.5 Kg,” Journal of Thoracic and Cardiovascular Surgery 137(5): 1163-1167; Marcheix, B., Lamarche, Y., Perrault, P., Cartier, R., Bouchard, D., and 3 others 2007. “Endovascular Management of Pseudo-aneurysms after Previous Surgical Repair of Congenital Aortic Coarctation,” European Journal of Cardiothoracic Surgery 31(6): 1004-1007; Serfontein, S. J. and Kron, I. L. 2002. “Complications of Coarctation Repair,” Seminars in Thoracic and Cardiovascular Surgery. Pediatric Cardiac Surgery Annual 5:206-211; Dodge-Khatami, A., Backer, C. L., and Mavroudis, C. 2000. “Risk Factors for Recoarctation and Results of Reoperation: A 40-year Review,” Journal of Cardiac Surgery 15(6): 369-377; Kino, K., Sano, S., Sugawara, E., Kohmoto, T., and Kamada, M. 1996. “Late Aneurysm after Subclavian Flap Aortoplasty for Coarctation of the Aorta,” Annals of Thoracic Surgery 61(4): 1262-1264; Hartmann, A. F. Jr., Goldring, D., Hernandez, A., Behrer, M. R., Schad, N., Ferguson, T., and Burfort, T. 1970. “Recurrent Coarctation of the Aorta after Successful Repair in Infancy,” American Journal of Cardiology 25(4): 405-410).

When the conformation of a defect makes it possible, blood flow bypassed directly from upstream to downstream of the defect such as a coarctation or an interruption is avoided nonincisionally, defects situated of the thoracic aorta appear downstream to the left subclavian artery. That is, where the constriction occurs where the left common carotid or left subclavian, for example, originate, blood is passed from the level of the aortic arch preceding to the level along the arch following the defect.

Depending upon which supraaortic branches are upstream to the constriction or interruption, bypass thus can alleviate the elevated pressure through the brachiocephalic, left common carotid, or the left subclavian arteries without at the same time reducing the blood supply. When situated further upsteam, bypasses can be connected to the supraaortic branches as well as downstream to these. Such bypasses can be passive, connected by simple side-entry jackets without the need for continuous or intermittent metering and control with vascular servovalve controlled by the implanted disorder control system.

Following repair of a coarctation using conventional methods, subsequent defects can arise due to the degenerate condition of the aortic wall. “Both endovascular and conventional open repair play important roles in the treatment of late complications after previous coarctation repair.”)(Beckmann, E. and Jassar, A. S. 2016. “Coarctation Repair-redo Challenges in the Adults: What to Do?,” Online, Journal of Visualized Surgery (Hong Kong, China) 4:76, also cited above in section 2a, entitled Background of the Invention. General) and weak points along suture lines, the segment has a propensity to fail and balloon outward as an aneurysm.

Various risks addressed below, an aneurysm accompanied by a coarctation can be treated with a stent endograft plus a bypass to protect against a failure of the endograft, such as restenosis, aortic wall complication, an endoleak, or collapse (Bai, J., Liu, Y., Jin, J., Li, J., Ji, X., and Qu, L. 2018. “Single-stage Endovascular Management of Complicated Thoracic Aorta Coarctation Concurrent with Aortic Arch Aneurysm Using a Novel Fenestration Device,” Journal of Thoracic Disease 10(4): 2474-2480; Nation, D. A. and Wang, G. J. 2015. “TEVAR [Thoracic Endovascular Aortic Repair]: Endovascular Repair of the Thoracic Aorta,” Seminars in Interventional Radiology 32(3): 265-271; Luijendijk, P., Bouma, B. J., Groenink, M., Boekholdt, M., Hazekamp, M. G., and 4 others 2012. “Surgical versus Percutaneous Treatment of Aortic Coarctation: New Standards in An Era of Transcatheter Repair,” Expert Review of Cardiovascular Therapy 10(12): 1517-1531; Tsukamoto, O., Seto, S., Moriya, M., and Yano, K. 2003. “Left Cervical Aortic Arch Associated with Aortic Aneurysm, Aortic Coarctation, and Branch Artery Aneurysm—A Case Report and Review,” Angiology 54(2): 257-260; Jonker, F. H., Schlosser, F., Geirsson, A., Sumpio, B. E., Moll, F. L., and Muhs, B. E. 2010. “Endograft Collapse after Thoracic Endovascular Aortic Repair,” Journal of Endovascular Therapy 17(6): 725-734; additional reference cited in the section below entitled Aneurysm and/or Coarctation Bypass Conduits).

Conventional means of repair carry risks and pose limitations which a dynamically responsive bypass can evade (Daye, D. and Walker, T. G. 2018. “Complications of Endovascular Aneurysm Repair of the Thoracic and Abdominal Aorta: Evaluation and Management,” Cardiovascular Diagnosis and Therapy 8 (Supplement 1): S138-S156; Torok, R. D., Campbell, M. J., Fleming, G. A., and Hill, K. D. 2015, Op cit.; Jonker, F. H., Schlosser, F., Geirsson, A., Sumpio, B. E., Moll, F. L., and Muhs, B. E. 2010, Op cit.). A collapse across a bypassed segment, for example, would seldom if ever have medical significance.

A conventional or diversion side-entry jacket and drugline just upstream to a lesioned or diseased segment along a substrate vascular or gastrointestinal ductus can deliver medication directly to the affected tissue. When a vessel is end-arterial, direct targeting allows the release of topical medication or post liver passthrough formulated medication at a higher dose than would be released into the general circulation. Recovery of a residue past the target is addressed in the antecedent patents specified above at the outset of this specification.

Upstream druglines in side-entry jackets, servovalves, of inline coupling jackets can serve mid- and postprocedurally to ameliorate the adverse tissue reaction foreign body interface between the bisphenol A and residual plasticizer-free polymeric material and tissue as well as any adverse reaction associated with sutures placed to secure an endoluminal stent-graft, for example. The same applies to sutures used to end-to-end or side-to-end anastomose a graft used to replace a segment along the vascular tree such as aneurysmal, along the gut as colitic, or otherwise diseased anywhere else during open surgery.

In reducing the inflammation caused by an interventionally or surgically introduced implement, an upstream jacket releasing a steroid, for example, affects the segment immediately downstream. The need for another jacket downstream to extract any residue or deliver a reversal agent should arise rarely if ever. Recovery of a drug can be accomplished by coupling it with a magnetically susceptible carrier molecule. Extraction and interdiction from continued flow through the circulatory system are addressed in U.S. Pat. No. 11,389,171, entitled Integrated System for the Infixion and Retrieval of Implants

A mesh woven using polyethylene terephthalate (Dacron®, Terylene®, woven Dacron® or Terylene® can be cut to form intricately shaped custom patches to replace aneurysmally outpouched tissue of the thoracic aorta; for example; however, when directly sutured to the aorta, the material fails to replace adjacent tissue no more structurally sound, and exceptionally, may provoke an adverse tissue reaction. Direct contact of native tissue with any foreign material can provoke an adverse reaction, some synthetics more problematic and some patients more sensitive than others.

For less extensive aneurysms of the ascending aorta in an adult without a congenital connective tissue disorder and for whom growth is not a factor, Dacron® mesh often provides a satisfactory repair (Cohen, O., Odim, J., De la Zerda, D., Ukatu, C., Vyas, R., Vyas, N., Palatnik, K., and Laks, H. 2007. “Long-term Experience of Girdling the Ascending Aorta with Dacron Mesh as Definitive Treatment for Aneurysmal Dilation,” Annals of Thoracic Surgery 83(2): S780-S790); however, even these may result in leaks necessitating application of the Cabrol technique for rechanneling of the extravasated blood back into the heart (Vyas, R., Mahmood, S., Syed, M. A., Alhazmi, L., and Grande, R. 2019. “Long-term Postoperative Complication of the Cabrol Procedure Presenting as Recurrent Anterior ST-Segment Elevation Myocardial Infarction,” CASE [Cardiovascular Imaging Case Reports] (Philadelphia, Pennsylvania). 35): 220-226; Chiu, P. and Miller, D. C. 2016. “Evolution of Surgical Therapy for Stanford Acute Type A Aortic Dissection,” Annals of Cardiothoracic Surgery (Hong Kong, China) 5(4): 275-295; Sran, S., Sran, M., Ferguson, N., and Makaryus, A. N. 2014. “Surgical Management of Ascending Aortic Aneurysm and Its Complications,” Case Reports in Vascular Medicine 2014:102605), and more complex extensive patches to cover debranching not covered by mere girdling, or wrapping, may require the addition of an endovascular stent, and the hybrid may still fail (Coselli, J. S., Spiliotopoulos, K., Preventza, O., de la Cruz, K. I., Amarasekara, H.4., and Green, S. Y. 2016. “Open Aortic Surgery after Thoracic Endovascular Aortic Repair,” General Thoracic and Cardiovascular Surgery 64(8): 441-449; Treasure, T. 2016. “A ‘Compare and Contrast Exercise: Wrapping versus Personalised External Aortic Root Support (PEARS),” Journal of Cardiothoracic Surgery 11(1): 104; Vallabhajosyula, P., Szeto, W. Y., Desai, N., Komlo, C., and Bavaria, J. E. 2013. “Type II Arch Hybrid Debranching Procedure,” Annals of Cardiothoracic Surgery (Hong Kong, China) 2(3): 378-386). The advantages posed by a jacket at the treatment site in allowing the direct targeting of medication stand regardless of the procedure employed to repair the defect.

While such authors as Cohen et al. 2007 (Op cit.) Milgalter et al., (Milgalter, E. and Laks, H. 1991. “Dacron Mesh Wrapping to Support the Aneurysmally Dilated or Friable Ascending Aorta,” Annals of Thoracic Surgery 52(4): 874-876), Tagarakis et al. (Tagarakis, G. I., Karangelis, D., Baddour, A. J., Daskalopoulos, M. E., Liouras, V. T., and 4 others 2010. “An Alternate Solution for the Treatment of Ascending Aortic Aneurysms: The Wrapping Technique,” Journal of Cardiothoracic Surgery 5:100), Arsan et al. (Arsan, S., Akgun, S., Kurtoglu, N., Yildirim, T., and Tekinsoy, B. 2004. “Reduction Aortoplasty and External Wrapping for Moderately Sized Tubular Ascending Aortic Aneurysm with Concomitant Operations,” Annals of Thoracic Surgery 78(3): 858-861; Plonek et al. (Plonek, T., Dumanski, A., Nowicki, R., and Kustrzycki, W. 2016. “Computed Tomography Angiography of Aorta Subjected to External Wrapping,” Journal of Cardiothoracic Surgery 11(1): 89; Plonek, T., Rylski, B., Dumanski, A., Siedlaczek, P., and Kustrzycki, W. 2015. “Biomechanical Analysis of Wrapping of the Moderately Dilated Ascending Aorta,” Journal of Cardiothoracic Surgery 10:106; Plonek, T., Dumanski, A., Nowicki, R., and Kustrzycki, W. 2015. “Single Center Experience with Wrapping of the Dilated Ascending Aorta,” Journal of Cardiothoracic Surgery 10:168; Plonek. T. 2014. “A Meta Analysis and Systematic Review of Wrapping of the Ascending Aorta,” Journal of Cardiac Surgery 29(6): 809-815), and others do not specify the use of highly porous Dacron® mesh (see, for example Tanabe. T., Kubo. Y., Hashimoto, M., Takahashi. T., Yasuda, K., and Sugie, S. 1980. “Wall Reinforcement with Highly Porous Dacron Mesh in Aortic Surgery” Annals of Surgery (Philadelphia, Pennsylvania) 191(4): 452-455), it is virtually certain that such material was used.

The inducement of atherosclerotic degeneration from compression of the aortic vasa vasorum and vasa nervorum and the elimination of exposure to the surrounding environment so that moisture can evaporate and gas exchange is cut off would likely have followed from the moment of placement, so that success would not have been forthcoming, even in elderly patients who might have expired from unrelated causes before the degenerative effects took effect (see, for example, De Meyer, G. R., Van Put. D. J., Kockx, M. M., Van Schil, P., Bosmans, R., and 4 others 1997, Op cit.; Tochii, M., Amano, K., Sakurai, Y., Ishikawa, H., Ishida, M., Higuchi, Y., and Takagi, Y. 2016. “Recurrence of Aneurysm of the Ascending Aorta after Patch Repair: The Fate of an Aortic Patch,” Annals of Vascular Disease 9(4): 322-325; Adam, D. J., Berce. M., Hartley, D. E., Robinson, D. A., and Anderson, J. L. 2007. “Repair of Recurrent Visceral Aortic Patch Aneurysm after Thoracoabdominal Aortic Aneurysm Repair with a Branched Endovascular Stent-graft,” Journal of Vascular Surgery (New York, New York) 45(1): 183-185).

An aortic arch or thoracic aorta replacement prosthesis made using the expandable structure and secured at either end with inline coupling jackets will allow a malformed or damaged portion of the arch or almost the entire thoracic aorta to be excised and replaced to include the arch and supraaortic branches. Related conventional methods for the treatment of dissections, aneurysms, pseudoaneurysms, coarctations, and interruptions of lesser extent are well documented in the literature (see, for example, Di Marco, L., Pantaleo, A., Leone, A., Murana, G., Di Bartolomeo, R., and Pacini. D. 2017. “The Frozen Elephant Trunk Technique: European Association for Cardio-Thoracic Surgery Position and Bologna Experience,” Korean Journal of Thoracic and Cardiovascular Surgery 50(1): 1-7; Pan, X. D., Li, B., Ma, W. G., Zheng. J., Liu, Y. M., and 3 others 2017. “Endovascular Repair of Residual Intimal Tear or Distal New Entry after Frozen Elephant Trunk for Type A Aortic Dissection,” Journal of Thoracic Disease 9(3): 529-536; Zhu, X., Li, Y., Li, Q., Luo. S., and Wu, Z. 2016. “A Simplified Arch Debranching Technique for Hybrid Repair of Complicated Type B Aortic Dissection,” Annals of Thoracic Surgery 102(1): e69-e72; Martens, A., Beckmann, E., Kaufeld, T., Umminger, J., Fleissner, F., and 5 others 2016. “Total Aortic Arch Repair: Risk Factor Analysis and Follow-up in 199 Patients,” European Journal of Cardiothoracic Surgery 50(5): 940-948; Faulds, J., Sandhu, H. K., Estrera, A. L., and Safi, H. J. 2016. “Minimally Invasive Techniques for Total Aortic Arch Reconstruction,” Methodist DeBakey Cardiovascular Journal 12(1): 41-44; Takagi, H. and Umemoto, T. 2016. “A Meta-Analysis of Total Arch Replacement with Frozen Elephant Trunk in Acute Type A Aortic Dissection,” Vascular and Endovascular Surgery 50(1): 33-46; Benrashid, E., Wang, H., Keenan, J. E., Andersen, N. D., Meza, J. M., McCann, R. L., and Hughes, G. C. 2016. “Evolving Practice Pattern Changes and Outcomes in the Era of Hybrid Aortic Arch Repair,” Journal of Vascular Surgery (New York, New York) 63(2): 323-331; Liu, L., Qin, C., Hou, J., Zhu, D., Zhang, B., Ma. H., and Guo, Y. 2016. “One-stage Hybrid Surgery for Acute Stanford Type A Aortic Dissection with David Operation, Aortic Arch Debranching, and Endovascular Graft: A Case Report,” Journal of Thoracic Disease 8(12): E1597-E1601; Suzuki, E., Fujita, D., Takahashi, M., Oba. S., and Nishimatsu, H. 2016. “Stem Cell-derived Exosomes as a Therapeutic Tool for Cardiovascular Disease,” World Journal of Stem Cells 8(9): 297-305; Shrestha, M., Krueger, H., Umminger, J., Koigeldiyev. N., Beckmann, E., Haverich, A., and Martens, A. 2015. “Minimally Invasive Valve Sparing Aortic Root Replacement (David Procedure) is Safe,” Annals of Cardiothoracic Surgery (Hong Kong, China) 4(2): 148-153; De Rango, P., Ferrer, C., Coscarella, C., Musumeci, F., Verzini, F., Pogany, G., Montalto, A., and Cao, P. 2015. “Contemporary Comparison of Aortic Arch Repair by Endovascular and Open Surgical Reconstructions,” Journal of Vascular Surgery (New York, New York) 61(2): 339-346; Lindblad, B., Bin Jabr. A., Holst, J., and Malina, M. 2015. “Chimney Grafts in Aortic Stent-grafting: Hazardous or Useful Technique? Systematic Review of Current Data,” European Journal of Vascular and Endovascular Surgery 50(6): 722-731; Esposito, G., Cappabianca, G., Bichi, S., Cricco, A., Albano, G., and Anzuini, A. 2015. “Hybrid Repair of Type A Acute Aortic Dissections with the Lupiae Technique: Ten-year Results,” Journal of Thoracic and Cardiovascular Surgery 149(2 Suppmentl): S99-104; Di Bartolomeo, R., Pantaleo, A., Berretta, P., Murana, G., Castrovinci, S., and 3 others 2015. “Frozen Elephant Trunk Surgery in Acute Aortic Dissection,” Journal of Thoracic and Cardiovascular Surgery 149(2 Supplement): S105-S109; Ma. W. G., Zheng. J., Sun, L. Z., and Elefteriades, J. A. 2015. “Open Stented Grafts for Frozen Elephant Trunk Technique: Technical Aspects and Current Outcomes,” Aorta (Stamford, Connecticut) 3(4): 122-135; Bin Jabr. A., Lindblad, B., Dias, N., Resch, T., and Malina, M. 2015. “Efficacy and Durability of the Chimney Graft Technique in Urgent and Complex Thoracic Endovascular Aortic Repair,” Journal of Vascular Surgery (New York, New York) 61(4): 886-894.e1: Rylski. B., Czemy, M., Südkamp, M., Russe, M., Siep. M., and Beyersdorf, F. 2015. “Fenestrated and Branched Aortic Grafts: Patency, Perioperative Mortality, and Spinal Ischemia” Deutsches Arzteblatt International 112(48): 816-822; Oskowitz, A. Z., Archie, M., Archie, M., and Quinones-Baldrich, W. 2015. “Hybrid Treatment of Aortic Arch Aneurysms,” Journal of Cardiovascular Surgery (Turin) 56(5): 719-728; Zhu, J. M., Qi. R. D., Chen, L., Liu, W., Li, C. N., Fan, Z. M., and Sun, L. Z. 2015. “Stented Elephant Trunk Procedure with Left Subclavian Artery Transposition for Acute Type B Dissection with Distal Arch Involvement,” Journal of Thoracic and Cardiovascular Surgery 150(5): 1160-1165; Maurel, B., Sobocinski, J., Spear, R., Azzaoui, R., Koussa, M., and 4 others 2015. “Current and Future Perspectives in the Repair of Aneurysms Involving the Aortic Arch,” Journal of Cardiovascular Surgery (Turin) 56(2): 197-215; Viswanathan, S., Agrawal. V., Parameshwarappa, S. K., Savlania, A., Kumar, S., and Madathipat, U. 2014. “Hybrid Strategy for Residual Arch and Thoracic Aortic Dissection following Acute Type A Aortic Dissection Repair,” Case Reports in Vascular Medicine 2014:165425; Shi, E., Gu. T., Yu, Y., Wang, C., Yu, L., Fang, Q., and Zhang, Y. 2014. “Simplified Total Arch Repair with a Stented Graft for Acute DeBakey Type I Dissection,” Journal of Thoracic and Cardiovascular Surgery 148(5): 2147-2154; Wu, H. B., Zhang, H., Wang, Z. W., Hu, R., Li, L. C., Zhang, M., and Hu, X. P. 2014. “Surgery for Acute Aortic Dissection Using the Chinese CRONUS [brand name for an open stented graft used in the frozen elephant trunk technique for repairing the thoracic aorta] Stented Elephant Trunk Technique: Experience with 252 Patients,” Journal of Thoracic and Cardiovascular Surgery 148(5): 2132-2138; Gunn, T. M., Amin, P. B., and Stamou S. C. 2014. “Hybrid Arch Debranching and Endograft Repair in a Patient with a Ruptured Thoracic Aortic Aneurysm,” Journal of Cardiac Surgery 29(2): 231-233; Bunger, C. M., Kische, S., Liebold, A., LeiBner, M., Glass, A., and 3 others 2013. “Hybrid Aortic Arch Repair for Complicated Type B Aortic Dissection,” Journal of Vascular Surgery (New York, New York) 58(6): 1490-1496; Cochennec. F., Tresson, P., Cross, J., Desgranges, P., Allaire, E., and Becquemin, J. P. 2013. “Hybrid Repair of Aortic Arch Dissections,” Journal of Vascular Surgery (New York, New York) 57(6): 1560-1567; Ikenaga, S., Mikamo, A., Kudo. T., Kurazumi, H., Suzuki, R., and Hamano, K. 2013. “Arch Translocation and the Intra-arch Elephant-trunk Technique with Collared Graft for Extended Chronic Dissecting Aortic Aneurysm,” Journal of Cardiothoracic Surgery 8:23: Lee, C. W., Beaver, T. M., Klodell, C. T. Jr., Hess, P. J. Jr., Martin, T. D., Feezor, R. J., and Lee, W. A. 2011. “Arch Debranching versus Elephant Trunk Procedures for Hybrid Repair of Thoracic Aortic Pathologies,” Annals of Thoracic Surgery 91(2): 465-471; Ramlawi, B. and Reardon, M. J. 2011. “Aortic Arch Debranching: Advanced and Hybrid Techniques,” Methodist DeBakey Cardiovascular Journal 7(3): 43-47; Johnson, P. T., Corl, F. M., Black, J. H., and Fishman, E. K. 2011. “The Elephant Trunk Procedure for Aortic Aneurysm Repair: An Illustrated Guide to Surgical Technique with CT Correlation,” American Journal of Roentgenology 197(6): W1052-W1059; Antoniou, G. A., El Sakka, K., Hamady, M., and Wolfe, J. H. 2010. “Hybrid Treatment of Complex Aortic Arch Disease with Supra-aortic Debranching and Endovascular Stent-graft Repair,” European Journal of Vascular and Endovascular Surgery 39(6): 683-690; Coselli, J. S. and Green. S. Y. 2009. Op cit above in section 2a: Schumacher, H., Von Tengg-Kobligk, H., Ostovic. M., Henninger. V., Ockert. S., Böckler, D., and Allenberg, J. R. 2006. “Hybrid Aortic Procedures for Endoluminal Arch Replacement in thoracic Aneurysms and Type B Dissections,” Journal of Cardiovascular Surgery (Turin, Italy) 47(5): 509-517; Safi, H. J., Miller, C. C. 3rd. Estrera, A. L., Huynh, T. T., Porat, E. E., Allen, B. S., and Sheinbaum, R. 2004. “Staged Repair of Extensive Aortic Aneurysms: Long-term Experience with the Elephant Trunk Technique,” Annals of Surgery (Philadelphia, Pennsylvania) 240(4): 677-685; Safi, H. J. Miller, C. C., Estrera, A. L., Huynh, T. T. T. Rubenstein, F. S., Huynh, Subramaniam, M. H., and Buja, L. M. 2001. “Staged Repair of Extensive Aortic Aneurysms. Morbidity and Mortality in the Elephant Trunk Technique,” Circulation 104(24): 2938-2942).

Repairs of the thoracic aorta requiring an open surgically placed or hybrid replacement graft made of or including nonreinforced polymer sheeting, usually nitinol reinforced woven polyethylene Dacron®, or Terylene®, to accomplish a repair for which a marketed stent such as the JOTEC E-vita Open Plus®, Vascutek Limited Thoraflex™ Hybrid, MicroPort Scientific Corporation Cronus™, and J Graftis is unavailable are sufficiently flexible to allow compliance with the pulse.

However, repair by such conventional focused means risks leaving behind adjacent tissue which even if strong at the time of the procedure might later become malacotic and predisposed to structural failure. Where a seamless expandable structure type prosthesis will grow with an infant, local repairs in an infant will eventually necessitate reoperation. With a prosthesis of the type indicated, nonsystemic hypertension can be counteracted, possibly alleviated before properly cured, by the direct release of antihypertensive medication into the affected prothesis. If hypertension persists, a tuned shunt as addressed below in section 2b(2)(h)(h1), entitled The ‘Tuned’ Shunt, and its subsidiary sections, and FIG. 22F, especially when added to the medication, will instate normotension.

Aortic arch stent-grafts likely more stably positioned, even these remain subject to migrate, with aortoiliac stent-grafts more decidedly lacking in positional retention and susceptible to the development of endoleaks: “Endovascular healing provides poor fixation of Dacron stent-grafts in humans. At present, fixation relies on the mechanical properties of the stent-grafts,” (Malina, M., Brunkwall, J., Ivancev, K., Jonsson, J., Malina, J., and Lindblad, B. 2000. “Endovascular Healing is Inadequate for Fixation of Dacron Stent-grafts in Human Aortoiliac Vessels,” Online, European Journal of Vascular and Endovascular Surgery (London, England) 19(1): 5-11).

In the succeeding two decades, the mechanical properties have probably been improved to an extent but not to the extent necessary, even in thoracic aortic stent-grafts (see, for example, Ma. Y., Siddiqui M. S., Farhan, S. A., Albuquerque, F. C., Larson, R. A., and 3 others 2023. “A Meta-analysis on the Effect of Proximal Landing Zone Location on Stroke and Mortality in Thoracic Endovascular Aortic Repair,” Journal of Vascular Surgery (New York, New York) S0741-5214(23) 01185-0; Deshpande, A. A., Pandey, N. N. Shaw, M., Kumar, S., Jagia, P., Sharma, S., and Choudhary, S. 2022. “Stent-graft Migration Post-endovascular Repair of Thoracic Aorta: A Retrospective Cohort Study,” Online, Indian Journal of Radiology and Imaging (Mumbai/[Bombay] India) 32(3): 324-331; Asenbaum, U., Schoder, M., Schwartz, E., Langs, G., Baltzer, P., and 4 others 2019. “Stent-graft Surface Movement after Endovascular Aneurysm Repair: Baseline Parameters for Prediction, and Association with Migration and Stent-graft-related Endoleaks,” Online, European Radiology (Berlin, Germany) 29(12): 6385-6395).

Extending the repair over the proximal margin of an open surgically placed woven polyethylene (Dacron®) nitinol reinforced shaped sheet graft rather than a transluminally positioned scaffold-reinforced endograft or stent-graft, can also be used to reduce an excessive outward excursion or bulging of the patch responsive to the pressure of the pulse to within normal limits. However, extension by entirely replacing the relevant segment with a seamless prosthesis is more dependable (see, for example, Marrocco-Trischitta, M. M., de Beaufort, H. W., Piffaretti, G., Bonardelli, S., Gargiulo, M., and 5 others 2020. “The Modified Arch Landing Areas Nomenclature Predicts Proximal Endograft Failure after Thoracic Endovascular Aortic Repair,” Online, European Journal of Cardiothoracic Surgery (Oxford, England) 2020 58(2): 309-318; Spadaccio, C., Nappi. F., Al-Attar, N., Sutherland, F. W., Acar. C., and 4 others 2016. “Old Myths, New Concerns: The Long-Term Effects of Ascending Aorta Replacement with Dacron® Grafts. Not All That Glitters is Gold,” Journal of Cardiovascular Translational Research 9(4): 334-342; Juszkat. R., Perek. B., Zabicki, B., Trojnarska, O., Jemielity, M., Staniszewski, R., Smoczyk, W., and Pukacki, F. 2013. “Endovascular Treatment of Late Thoracic Aortic Aneurysms after Surgical Repair of Congenital Aortic Coarctation in Childhood,” Online, PLoS [Public Library of Science] One 8(12): e83601; Tshomba, Y., Bertoglio, L., Marone, E. M., Melissano, G., and Chiesa, R. 2008. “Visceral Aortic Patch Aneurysm after Thoracoabdominal Aortic Repair: Conventional vs Hybrid Treatment,” Journal of Vascular Surgery (New York, New York) 48(5): 1083-1091; Tshomba, Y., Melissano, G., Civilini. E., Setacci, F., and Chiesa, R. 2005. “Fate of the Visceral Aortic Patch after Thoracoabdominal Aortic Repair,” European Journal of Vascular and Endovascular Surgery 29(4): 383-389). U.S. Pat. No. 11,759,186 shows an example of jacket position FIG. 21 positioned about the ascending aorta.

Made of tough but pliant polyetheretherketone or polypropylene and replacing not a delimited area along the wider structure but rather much if not all of the structure, the prosthesis described herein is unsusceptible to dehiscence or rupture, and is durable enough to withstand a direct impact. In contrast, rupture of a secondary abdominal aortic aneurysm despite earlier placement of a nitinol reinforced Dacron® mesh endovascular stent-graft to repair an earlier aneurysm, for example, is not due to fatigue from the blood pressure but rather a gradual weakening of the aortic wall, endoleak, migration, aortoenteric fistula, and/or extension of the rupture past the stent-graft margin or cuff, often due to an endoleak (see, for example, Okada, M., Handa, N., Onohara, T., Okamoto, M., Yamamoto. T., and 19 others 2016. “Late Sac Behavior after Endovascular Aneurysm Repair for Abdominal Aortic Aneurysm,” Annals of Vascular Diseases 9(2): 102-107; Dingemans, S. A., Jonker, F. H., Moll, F. L., and van Herwaarden, J. A. 2016. “Aneurysm Sac Enlargement after Endovascular Abdominal Aortic Aneurysm Repair,” Annals of Vascular Surgery 31:229-238; Takebayashi, S., Hirota, J., Mori, K., Shuto. T., Okamoto, K., Sato, A., Wada. T., Anai. H., and Miyamoto, S. 2016. “Unique Technique for Open Surgical Repair after Failed Endovascular Aneurysm Repair with Proximal Anastomoses,” Annals of Vascular Diseases (Tokyo, Japan) 9(2): 120-124; Antoniou, G. A., Georgiadis, G. S., Antoniou, S. A., Neequaye, S., Brennan, J. A., Torella, F., and Vallabhaneni, S. R. 2015. “Late Rupture of Abdominal Aortic Aneurysm after Previous Endovascular Repair: A Systematic Review and Meta-analysis,” Journal of Endovascular Therapy 22(5): 734-744; Klonaris, C., Lioudaki, S., Katsargyris, A., Psathas, E., Kouvelos, G., Doulaptsis, M., Verikokos, C., and Kouraklis, G. 2014. “Late Open Conversion after Failed Endovascular Aortic Aneurysm Repair,” Journal of Vascular Surgery (New York, New York) 59(2): 291-297; Cho, J. S. 2014. “Ruptured Abdominal Aortic Aneurysm with Antecedent Endovascular Repair of Abdominal Aortic Aneurysm,” Vascular Specialist International 30(1): 1-4; Szmidt, J., Galazka, Z., Rowinski, O., Nazarewski, S., Jakimowicz, T., Pietrasik, K., Grygiel, K., and Chudzinski, W. 2007. “Late Aneurysm Rupture after Endovascular Abdominal Aneurysm Repair,” Interactive Cardiovascular and Thoracic Surgery 6(4): 490-494; references pertaining to thoracic aneurysms and/or coarctations are provided below the section below entitled Aneurysm and/or Coarctation Bypass Conduits).

Combined open and endovascular repair to include the application of a perivascular wrap of highly porous reinforced woven Dacron® mesh at the same time as the original stent-graft is inserted would likely reduce the incidence of failure due to the foregoing causes, but-almost certainly because limitation to an endoluminal procedure is preferred and the failure rate is relatively low-such a double approach, even as a revision, is unjustified, and while simultaneous abdominal aortic replacement or open reconstruction and thoracic stent-graft placement does appear in the literature, combined transluminal and perivascular wrapping does not.

However, the placement of a side-entry diversion jacket upstream of the stent-graft can justify such a combined procedure. With or without the concurrent placement of a perforated perivascular wrap, the addition of a side-entry jacket allows the direct targeting of suture line maintenance and if necessary, antimicrobial drugs to the site of a stent-graft that becomes infected (see, for example, Eldin, C., Mailhe, M., Lions, C., Carrieri. P., Safi, H., Brouqui, P., and Raoult, D. 2016. “Treatment and Prophylactic Strategy for Coxiella burnetii Infection of Aneurysms and Vascular Grafts: A Retrospective Cohort Study,” Medicine (Baltimore) 95(12): e2810; Setacci, C., Chisci, E., Setacci, F., Ercolini, L., de Donato, G., Troisi, N., Galzerano, G., and Michelagnoli, S. 2014. “How to Diagnose and Manage Infected Endografts after Endovascular Aneurysm Repair,” Aorta (Stamford, Connecticut) 2(6): 255-264; Gonzalez-Del Vecchio, M., Vena, A., Valerio, M., Marin, M., Verde, E., Muñoz, P., and Bouza, E. 2014. “Coxiella burnetii Infection in Hemodialysis and Other Vascular Grafts,” Medicine (Baltimore) 93(24): 364-371; Cinal, C. S., Arena, G. O., Fiture, A. O., Clase, C. M., and Doobay, B. 2001. “Ruptured Mycotic Thoracoabdominal Aortic Aneurysms: A Report of Three Cases and a Systematic Review,” Journal of Vascular Surgery (New York, New York) 33(4): 861-867). A side-entry diversion jacket or vascular servovalve of a ‘tuned’ shunt as described below in section 2b(2)(h)(h1), entitled The ‘Tuned’ Shunt, and its subsidiary section and

FIG. 22F can not only divert a portion of the flow through the aorta into other vessels around an aneurysmally or otherwise affected segment and return the blood through a second side-entry jacket along the lower abdominal aorta restricting the lowering of the blood pressure to the affected area, for example, but its drugline or druglines can directly target medication to the affected area.

Moreover, following open repair of an aneurysm, a stent-graft or an outer wrap protects against the secondary formation of an aortoenteric, aortoduodenal, or aortoesophageal fistula, often associated with infection that a side-entry or side-entry diversion jacket can ameliorate if not eradicate, thus avoiding the need for open revision (see, for example, Nehme, F., Rowe, K., Munguti. C., and Nassif. 1. 2017. “A Rare Cause of Primary Aortoenteric Fistula: Streptococcus parasanguinis Aortitis,” Case Reports in Gastrointestinal Medicine 2017:9087308; Okwara, C. J., Petrasek, J., Gibson, M., and Burstein, E. 2016. Secondary Aortoesophageal Fistula Associated with Aneurysmal Graft Infection by Coxiella bumnetiid, “ACG [American College of Gastroenterology] Case Reports Journal 3(3): 169-171; Karthaus, E. G., Post, I. C., and Akkersdijk, G. J. 2016. “Spontaneous Aortoenteric Fistula Involving the Sigmoid: A Case Report and Review of Literature,” International Journal of Surgery Case Reports 19:97-99; Gulcu, A., Gezer, N. S., Uğurlu, S. B., and Goktay, A. Y. 2016. “An Aortoenteric Fistula Arising after Endovascular Management of a Mycotic Abdominal Aortic Aneurysm Complicated with a Psoas Abscess,” Iranian Journal of Medical Sciences 41(4): 350-353; Ho, S., Liu, B., Loya, R., and Koury, I. 2016. “Primary Aortoenteric Fistula: A Rare Case of a Massive Gastrointestinal Bleed,” Online, Cureus (Palo Alto, California) 8(9): e 766; Malik, M. U., Ucbilek, E., and Sherwal, A. S. 2015. “Critical Gastrointestinal Bleed Due to Secondary Aortoenteric Fistula,” Journal of Community Hospital Internal Medicine Perspectives 5(6): 29677; Tagowski, M., Vieweg, H., Wissgott. C., and Andresen, R. 2014. “Aortoenteric Fistula as a Complication of Open Reconstruction and Endovascular Repair of Abdominal Aorta,” Radiology Research and Practice 2014:383159; Zaki, M., Tawfick, W., Alawy, M., ElKassaby, M., Hynes, N., and Sultan, S. 2014. “Secondary Aortoduodenal Fistula Following Endovascular Repair of Inflammatory Abdominal Aortic Aneurysm Due to Streptococcus anginosus Infection: A Case Report and Literature Review,” International Journal of Surgery Case Reports 5(10): 710-713; Xi. E. P., Zhu, J., Zhu, S. B., and Zhang, Y. 2014. “Secondary Aortoesophageal Fistula after Thoracic Aortic Aneurysm Endovascular Repair: Literature Review and New Insights Regarding the Hypothesized Mechanisms,” International Journal of Clinical and Experimental Medicine 7(10): 3244-3252; Lee, C. W., Chung, S. W., Song. S., Bae. M. J., Huh, U., and Kim, J. H. 2012. “Double Primary Aortoenteric Fistulae: A Case Report of Two Simultaneous Primary Aortoenteric Fistulae in One Patient,” Korean Journal of Thoracic and Cardiovascular Surgery 45(5): 330-333; Gavens, E., Zaidi, Z., Al-Jundi, W., and Kumar, P. 2011. “Aortoesophageal Fistula after Endovascular Aortic Aneurysm Repair of a Mycotic Thoracic Aneurysm,” International Journal of Vascular Medicine 2011:649592; Saratzis, N., Saratzis, A., Melas, N., Ktenidis, K., and Kiskinis. D. 2008. “Aortoduodenal Fistulas after Endovascular Stent-graft Repair of Abdominal Aortic Aneurysms: Single-center Experience and Review of the Literature,” Journal of Endovascular Therapy 15(4): 441-448; Chaudhary, N. A., Saymeh, L., Vongthavaravat. V., and Harty, R. F. 2003. “Primary Aortoenteric Fistula to Jejunum: A Case Report,” Journal of the Oklahoma State Medical Association 96(11): 519-521).

When a side-entry jacket cannot be positioned just upstream to the patch or stent-graft, as with an aortic root repair, Bentall procedure, or placement of a valve graft, the side-entry diversion jacket or servovalve when it is desired to adjust the flow rate of the blood and therewith the uptake gradient of jacket-infused drug delivery—is positioned toward the terminus of the superior or inferior vena cava (see, for example, Dai, X. F., Fang, G. H., Yan, L. L., Dong, Y., Zhang, Gd. C., Xu, Z., and Chen, L. W. 2021. “Patch Neointima Technique in Acute Type A Aortic Dissection: Midterm Results of 147 Cases,” Annals of Thoracic Surgery (Amsterdam, North Holland) 112(1): 75-82; Vricella, L. A. and Cameron, D. E. 2017. “Valve-sparing Aortic Root Replacement in Pediatric Patients: Lessons Learned over Two Decades,” Seminars in Thoracic and Cardiovascular Surgery. Pediatric Cardiac Surgery Annual 20:56-62; Hillebrand, J., Alshakaki, M., Martens, S., and Scherer, M. 2017. “Minimally Invasive Aortic Root Replacement with Valved Conduits through Partial Upper Sternotomy,” [in press] Thoracic and Cardiovascular Surgery February 15; Nguyen, T. C., Thourani, V. H., Pham, J. Q., Zhao, Y., Terwelp, M. D., Balan, P., and 5 others 2017. “Traditional Sternotomy versus Minimally Invasive Aortic Valve Replacement in Patients Stratified by Ejection Fraction,” Innovations (Philadelphia) 12(1): 33-40; David, T. E. 2016. “Aortic Valve Sparing in Different Aortic Valve and Aortic Root Conditions,” Journal of the American College of Cardiology 68(6): 654-664; Di Marco, L., Pacini, D., Pantaleo, A., Leone, A., Barberio, G., Marinelli, G., and Di Bartolomeo, R. 2016. “Composite Valve Graft Implantation for the treatment of Aortic Valve and Root Disease: Results in 1045 Patients,” Journal of Thoracic and Cardiovascular Surgery 152(4): 1041-1048; Bowdish, M. E., Hui, D. S., Cleveland, J. D., Mack, W. J., Sinha. R., and 6 others 2016. “A Comparison of Aortic Valve Replacement Via an Anterior Right Minithoracotomy with Standard Sternotomy: A Propensity Score Analysis of 492 Patients,” European Journal of Cardiothoracic Surgery 49(2): 456-463; Yan, T. D. 2016. “Mini-Bentall Procedure: The “French Cuff” Technique,” Annals of Thoracic Surgeryl01(2): 780-782; Yan, T. D. 2015. “Mini-Bentall Procedure,” Annals of Cardiothoracic Surgery (Hong Kong, China) 4(2): 182-190; Gaudino, M., Lau, C., Munjal, M., Avgerinos, D., and Girardi, L. N. 2015. “Contemporary Outcomes of Surgery for Aortic Root Aneurysms: A Propensity-matched Comparison of Valve-sparing and Composite Valve Graft Replacement,” Journal of Thoracic and Cardiovascular Surgery 150(5): 1120-1129; Malaisrie, S. C., Bamhart, G. R., Farivar, R. S., Mehall, J., Hummel, B., and 17 others 2014. “Current Era Minimally Invasive Aortic Valve Replacement: Techniques and Practice,” Journal of Thoracic and Cardiovascular Surgery 147(1): 6-14; Maddalo, S., Beller, J., and DeAnda, A. 2014. “A Bentall is Not a Bentall is Not a Bentall: The Evolution of Aortic Root Surgery,” Aorta (Stamford, Connecticut) 2(5): 169-178; Tsai, H. C., Chen, I. M., Hsieh, Y. C., Shih, C. C., Chen, P. L., Chen, W. Y., Weng, Z. C., and Chang, H. H. 2013. “Concomitant Bentall Operation Plus Aortic Arch Replacement Surgery,” Journal of the Chinese Medical Association 76(2): 88-94; Heo, W., Min, H. K., Kang. D. K., Jun, H. J., Hwang, Y. H., Chen, L. W., Dai, X. F., Wu, X. J., and Zhang, G. C. 2013. “Valve-sparing Root Replacement with Root Reduction Plasty and Patch Neointima Placement,” Annals of Thoracic Surgery 95(4): 1459-1461; Urbanski, P. P., Hijazi, H., Dinstak, W., and Diegeler, A. 2013. Valve-sparing Aortic Root Repair in Acute Type A Dissection: How Many Sinuses Have to be Repaired for Curative Surgery?. “European Journal of Cardiothoracic Surgery 44(3): 439-444; Choi, J. H., and Wi, J. H. 2013. “A Modified Root Reinforcement Technique for Acute Aortic Dissection with a Weakened Aortic Root: A Modified Florida Sleeve Technique and Two Cases Report,” Journal of Cardiothoracic Surgery 8:203; Wartono, D. A. 2013. “Aortic Valve-sparing Operation in Aortic Root Aneurysms: Remodeling or Reimplantation? Interactive Cardiovascular and Thoracic Surgery available at https://www.slideshare.net/dickyawartono/aortic-valve-sparring-root-replacement; David, T. E. 2012. “Aortic Valve Sparing Operations: A Review,” Korean Journal of Thoracic and Cardiovascular Surgery 45(4): 205-212; Chen, L. W., Wu. X. J., Li, Q. Z., and Dai, X. F. 2012. “A Modified Valve-sparing Aortic Root Replacement Technique for Acute Type A Aortic Dissection: The Patch Neointima Technique,” European Journal of Cardiothoracic Surgery 42(4): 731-733; Rahnavardi, M., Yan, T. D., Bannon, P. G., and Wilson, M. K. 2011. “Aortic Valve-sparing Operations in Aortic Root Aneurysms: Remodeling or Reimplantation?,” Interactive Cardiovascular and Thoracic Surgery 13(2): 189-197; Nezic, D., Cirkovic, M., Knezevic, A., and Jovic, M. 2008. “Modified Bentall Procedure—‘A Collar Technique’ to Control Bleeding from Coronary Ostia Anastomoses,” Interactive Cardiovascular and Thoracic Surgery 7(4): 709-711).

Side-entry diversion jackets can provide directly targeted drug support to a graft whether synthetic, an autograft, or allograft (see, for example, Nappi, F., Nenna, A., Spadaccio. C., and Chello, M. 2017. “Pulmonary Autograft in Aortic Position: Is Everything Known?,” Translational Pediatrics 6(1): 11-17; Spadaccio, C., Nappi, F., Al-Attar, N., Sutherland, F. W., Acar. C., and 4 others 2016, Op cit.: Agaimy, A., Ben-Izhak, O., Lorey, T., Scharpf, M., and Rubin, B. P. 2016. “Angiosarcoma Arising in Association with Vascular Dacron® [Invista, Wichita, Kansas brand of woven polyethylene terephthalate] Grafts and Orthopedic Joint Prostheses: Clinicopathologic, Immunohistochemical, and Molecular Study,” Annals of Diagnostic Pathology 21:21-28; Okada, M., Takeuchi, E., Mori, Y., Ichihara, S., Usui, A., and Ueda, Y. 2004. “An Autopsy Case of Angiosarcoma Arising Around a Woven Dacron® Prosthesis after a Cabrol Operation,” Journal of Thoracic and Cardiovascular Surgery 127(6): 1843-1845; Khaira, H. S. and Vohra, H. 2002. “True Aneurysm in a Femoro-popliteal Dacron® Graft-A Case Report and Literature Review,” Cardiovascular Surgery (London) 10(6): 644-646; Ben-Izhak. O., Vlodavsky, E., Ofer, A., Engel, A., Nitecky, S., and Hoffman, A. 1999. “Epithelioid Angiosarcoma Associated with a Dacron® Vascular Graft,” American Journal of Surgical Pathology 23(11): 1418-1422; Wilson, S. E., Krug. R., Mueller, G., and Wilson, L. 1997. “Late Disruption of Dacron® Aortic Grafts,” Annals of Vascular Surgery 11(4): 383-386; Raso, A. M., Rispoli, P., Castagno, P. L., Muncinelli, M., Maggio, D., and Sandrone, N. 1993. “Malignant Fibrohistiocytoma7b Arising at a Femoral Dacron® Vascular Prosthesis. Case Report and Review of the Literature,” (in Italian with abstract in English at Pubmed), Minerva Chirurgica 48(20): 1227-1232; Weiss, W. M., Riles. T. S., Gouge. T. H., and Mizrachi, H. H. 1991. “Angiosarcoma at the Site of a Dacron® Vascular Prosthesis: A Case Report and Literature Review,” Journal of Vascular Surgery (New York, New York) 14(1): 87-91; Paterson, H. S., Meredith, D. J., and Craddock, D. R. 1989. “Malignant Fibrous Histiocytoma Associated with a Dacron® Vascular Prosthesis,” Annals of Thoracic Surgery 47(5): 772-774; Magee, H. R. and Teague, C. A. 1988. “Late Rupture of Knitted Velour Dacron® Arterial Prostheses,” Australian and New Zealand Journal of Surgery 58(7): 549-553; Osborne, D. R. and Hobbs, K. E. 1981. “Dacron® Graft Erosion of the Duodenum: A Complication of Interposition Mesocaval Shunt Operations,” British Journal of Surgery 68(7): 483-484; Smith, R. B. 3rd, Warren, W. D., Salam, A. A., Millikan, W. J., Ansley, J. D., and 3 others 1980. “Dacron® Interposition Shunts for Portal Hypertension. An analysis of Morbidity Correlates,” Annals of Surgery (Philadelphia, Pennsylvania) 192(1): 9-17).

Alternatively, a fully pulse compliant miniaturized nonjacketing side-entry connector as shown in U.S. Pat. No. 11,013,858, entitled Nonjacketing Side-entry Connectors and Prosthetic Disorder Response Systems, can be affixed to the outside of the pericardium over the left ventricle. The drug or drugs delivered then pass up with the ejection to wash over any sutures or synthetic-cardiac tissue interfaces at the aortic root, ascending aorta, and arch, the descending aorta affording direct placement. With a side-entry diversion jacket fastened to the superior or inferior vena cava or a nonjacketing side-entry connector fastened to the left ventricle, drug delivery is through a catheter entered through a scarcely noticeable subdermally placed button-sized port connected by the side-entry jacket or connector.

A vascular servovalve in a disorder response system feedback loop can adjust the rate of blood flow through and/or downstream to the treatment site accurately with or without shunting (Gelman, S, 1995. “The Pathophysiology of Aortic Cross-clamping and Unclamping,” Anesthesiology 82(4): 1026-1060). In the bloodstream, continuously variable adjustment of the luminal flow-through volume by the diversion chute may impart turbulence that requires the concurrent delivery of an anticoagulant such as a low molecular weight heparin through a valve drugline to counteract.

Where a body surface port to replenish implanted drug reservoirs such as shown in FIGS. 26A and 26B includes entry holes to treat one or more comorbid conditions, the entry holes for these are positioned subdermally in front of the body surface port backplate, part number 100. Since the extent of chute deployment is remotely controlled by the disorder system controller on the basis of sensor feedback, wound closure can proceed without any additional steps. The side-entry diversion jacket and line can be left in place, allowing adjustments to be made at a later date. When continued use will not be necessary, the anticoagulant is reduced if not stopped, and the jacket and line removed endoscopically. The body surface port will often remain in use for collateral treatment; if not, the surface port is removed.

Anticoagulation is stopped before removing the jacket. If the entry perforation made by the trepan during placement is 1.0 millimeter in diameter, withdrawal of the jacket will result in only brief extravasation, and the slight seepage should spontaneously be cut off by clot. If there is concern that the clot might embolize or the anticoagulant prohibits prompt clotting, then a drop of a surgical cement is used to wet the aperture. Chitosan allows withdrawal of the jacket even while heparin continues to pass. An entry hole larger than 1.0 millimeter is wetted with a topical nonallergenic hemostatic agent, or coagulant, such as a zeolite, chitosan, kaolin, styptic pencil (anhydrous aluminum sulfate), resorbable microfibrillar collagen, desmopressin, aminocaproic, or tranexamic acid is applied.

Unlike drug eluting or bioresorbable stents with only a limited amount of the drug available, the jacket can deliver one or more drugs indefinitely (see, for example, Sotomi, Y., Onuma, Y., Collet, C., Tenekecioglu, E., Virmani, R., Kleiman, N. S., and Serruys, P. W. 2017. “Bioresorbable Scaffold: The Emerging Reality and Future Directions,” Circulation Research 120(8): 1341-1352; Rizik, D. G., Hermiller, J., Simonton, C. A., Klassen, K. J., and Kereiakes, D. J. 2017. “Bioresorbable Vascular Scaffolds for the Treatment of Coronary Artery disease: What Have We Learned from Randomized-controlled Clinical Trials?,” Coronary Artery Disease 28(1): 77-89; Spadaccio, C., Nappi, F., Al-Attar, N., Coccia, R., Perluigi, M., and Di Domenico, F. 2017. “Current Developments in Drug Eluting Devices: Introductory Editorial: Drug-Eluting Stents or Drug-Eluting Grafts? Insights from Proteomic Analysis,” Drug Target Insights 10 (Supplement 1): 15-19; Lipinski, M. J., Escarcega, R. O., Baker, N. C., Benn, H. A., Gaglia, M. A. Jr., Torguson, R., and Waksman, R. 2016. “Scaffold Thrombosis after Percutaneous Coronary Intervention with ABSORB [brand name] Bioresorbable Vascular Scaffold: A Systematic Review and Meta-Analysis,” Journal of the American College of Cardiology. Cardiovascular Interventions 9(1): 12-24; Tanaka, A., Ruparelia, N., Kawamoto, H., Sticchi, A., Figini, F, and 5 others 2016. Clinical Outcomes Following Target Lesion Revascularization for Bioresorbable Scaffold Failure,” Catheterization and Cardiovascular Interventions 87(5): 832-836; Tenekecioglu, E., Bourantas, C., Abdelghani, M., Zeng, Y., Silva, R. C, and 5 others 2016. “From Drug Eluting Stents to Bioresorbable Scaffolds; to New Horizons in PCI [percutaneous intervention],” Expert Review of Medical Devices 13(3): 271-286; Wayangankar, S. A. and Ellis, S. G. 2015. “Bioresorbable Stents: Is This Where We are Headed?,” Progress in Cardiovascular Diseases 58(3): 342-355; Franzone, A., Stabile, E., Trimarco, B., and Esposito, G. 2015. “Peripheral Drug-eluting Technology,” Cardiology Clinics 33(1): 151-162; Scalone, G., Brugaletta, S., Gomez, O., Otsuki, S., and Sabate, M. 2015. “Bioresorbable Scaffolds: Focus on Vascular Response and Long-term Safety,” Panminerva Medica 7(1): 1-13).

When in order to expedite healing, the need exists to further reduce bulging of a patch thereby more aligning the arterial wall with normal hemodynamic responsivity, a diversion rather than an ordinary side-entry jacket is used to gradually adjust the cross-sectional area of the aorta, hence the pressure, until the excursion is reduced to normal. If necessary to achieve normal perfusion, a compensatory nitrovasodilator, glycerol trinitrate (nitroglycerin) or diethylene glycol dinitrate, for example, is included among the drugs.

Equivalent placement, that is, upstream to an endoluminal graft, endograft prosthesis, or stent-graft is placed to remedy an abdominal aortic aneurysm, for example. While less likely than with direct synthetic-to-tissue contact, the jacket-foam interface can be medicated to reduce any adverse tissue reaction by feeding a drugline into a flexible fine or capillary tube caliber catheter with side perforations—a tiny “soaker hose”—run just beneath the foam.

### Standardized Inline Aortic Arch Aneurysm, Coarctation, or Interruption Replacement Prosthesis

Existing means for the repair of a congenital or acquired defect of the aortic arch tend to be lacking in dependability in a part of the body where failure without immediate repair can result in death. For a neonate or infant, the inability of the repair to expand with growth means that multiple revisions may be necessary. Despite experience to the contrary, a revision must prove durable, and in a seriously ill baby unprepared to withstand repeated operations, if the underlying defect had not resulted in death, then it is little less likely that a failed operation will. The practical implementation of a type prosthesis to serve as replacements for major vessels which due to infection or genetic defect, for example, are prone to fail in a growing child is addressed below in section 2b(2)(f), entitled Expandable Structure to Accommodate Growth and Self-adjust in Size in Vascular Segment Prostheses, Lines, and Confluence Conduits Placed in Children.

In the repair of an aneurysm, coarctation, or interruption along the aortic arch, connection with inline coupling jackets of a standardized prosthesis as described below in section 2b(2)(f), eliminates the sideways protrusion of side-entry jacket and vascular servovalves sidestem, part number 19 in the drawing figures. This provides a fit nonintrusive to neighboring tissue while retaining the advantages otherwise provided by side-entry in migration and leak-free stitchless connection without the risks of dehiscence and rupture or the risk of migration associated with endostents. As to patch aortoplasty, “After repair of coarctation using Dacron® patch aortoplasty, the risk for aneurysm formation in the area of repair and death from rupture is extremely high,” (Cramer, J. W., Ginde, S. Bartz P. J., Tweddell, J. S., Litwin, S. B., and Earing, M. G. 2013 Op cit. above in section 2a).

And as to stent-grafts or endostents, an inline segment replacing prosthesis as a standardized inline aneurysm, coarctation, or interrupted aortic arch replacement, is inherently unsusceptible not only to migration but to the development of an endoleak, both of which could result in a rupture as the result of “ . . . poor proximal, distal, or junctional fixation of the stent-graft . . . ” (Zarins, C. K., White, R. A., Moll, F. L., Crabtree, T., Bloch, D. A., with 3 others and 19 collaborators 2001. “The AneuRx Stent-graft: Four-year Results and Worldwide Experience 2000,” Journal of Vascular Surgery (New York, New York) 33(2): S135-S145, page S143)(see also, Lee, H. K., Chung, S. Y., Kim, J. K., Yoo, S. H., and Choi, S. J. 2014. “Changes in Suprarenal and Infrarenal Aortic Angles after Endovascular Aneurysm Repair,” Online, Annals of Surgical Treatment and Research (Seoul, South Korea) 87(4): 197-202).

In adult patients, transluminal placement can reduce operative trauma but has its own complications and results in outcomes little if at all better than open surgical repair. The same can be said for hybrid repairs such as a frozen elephant trunk, of which the hazards are prominent in the minds of practitioners (see, for example, Lee, G, Elbatamy, M., Shimamura, J., Dagenais, F., Peterson, M. D., Ouzounian, M., and Chu, M. W. A. 2022. “Hybrid Arch Frozen Elephant Trunk Repair for Acute Type A Aortic Dissection: Extra-anatomic Subclavian Reconstruction,” Multimedia Manual of Cardio-Thoracic Surgery (Windsor, England). Adequate design of an alternative method of repair, here an inline coupling jacket-secured tie-line type prosthesis, begins with avoiding the well-established causes for vulnerability and eventual failure in current methods of repair.

In a neonate, time to develop collateral circulation as occurs in a patient born with a less pronounced congenital coarctation who was able to survive beyond the postnatal period is denied, making early repair of the defect imperative (see, for example, Agasthi, P., Pujari, S. H., Tseng, A., Graziano, J. N., Marcotte, F. and 4 others 2020. “Management of Adults with Coarctation of Aorta,” World Journal of Cardiology 12(5): 167-191). Critically for ensuing development in a neonate, repair thus can be performed much earlier than any conventional procedure used in an adult, and because the prosthesis is expandable, it can last far longer, allowing the neonate time to fully recover before an improbable need for revision arises.

For these reasons, a standardized inline seamless prosthesis such as an aortic neoarch to replace a congenitally defective aortic arch as descried above early in section 2a, entitled Background of the Invention. General, and below in section 2b(2)(f), entitled Expandable Structure to Accommodate Growth and Self-adjust in Size in Vascular Segment Prostheses, Lines, and Confluence Conduits Placed in Children, will allow the initiation of normal circulation and therefore development.

With circumferential flats 233 alternated with vaults 234 as shown specifically in FIG. 36F but also shown in FIGS. 15C, 15D, and 36A, the prosthetic neoarch is expandable to accommodate growth, and therewith, prevent the lack of growth of tissue in neighboring levels of the native aortic arch the eradication of a defect that would otherwise prevent normal development, much less prevent the death of a newborn affected with a defect which left untreated would almost certainly result in this consequence. Such a prosthesis limited to the aortic arch portion of the thoracic aorta assumes that the patient has no congenital connective tissue defect that would allow defects such as an aneurysm to appear aside from the neoarch as documented above in this section.

If so, the drugline 8 of the inlet inline inner half-cylinder coupling half-jacket shown in FIG. 36E is placed to enable the implanted automatic disorder response to cause the release of substances specified above in this section to strengthen the downstream, or post-neoarch, intima with an independent side-entry jacket positioned upstream to the neoarch to cover the upstream segment of the thoracic aorta. If a connective tissue is present, a prosthesis is chosen to replace the thoracic aorta, and exceptionally the aorta in its entirety.

When the loss of endothelium is this extensive, its many functions require that supplements likewise internally infused by the control system be released to compensate for these functions. Provided the number of strengthening and endothelial function replacing substances is not prohibitive and the patient is prescription adherent, some if not all of these substances can be self-administered aside from the control system, which is then freed to deal with comorbidities if present.

### Off Pump Surgery of the Aorta

While off pump surgery related to the heart is limited to aorta-extraluminal (non-aortotomy, aorta non-transecting) procedures such as a rare aneurysm plication but otherwise coronary artery bypass grafting, it cannot be used for the nonmanipulative removal and replacement of a segment along the aorta as intended using the means described this and its subsidiary sections (see, for example, Coronella, G., Pepino, P., Pezzella, E., Contaldo, A., Provenzano, R., and 5 others 2013. “Off Pump Reduction Aortoplasty in Ascending Aortic Aneurysm,” Online, [BioMed Central] Journal of Cardiothoracic Surgery (London, England) 8 (Supplement 1): 014).

Placed off-pump under regional anesthesia without interruption in the flow of blood through the aorta, the additional serious complications risked with cardiopulmonary bypass would exclude patients too frail to risk these (see, for example, section on complications in Sarkar, M. and Prabhu, V. 2017. “Basics of Cardiopulmonary Bypass,” Indian Journal of Anaesthesia (Mumbai [Bombay], India) 61(9): 760-767)(see also section entitled Is Cardiopulmonary Bypass Safe? in Cleveland Clinic 2023. “Cardiopulmonary Bypass,” Online at What is Cardiopulmonary Bypass? (clevelandclinic.org); Roberts, A., Duncan, E. C., Hargrave, P., Kingery, D. R., Bares., J, Horstemeyer, D. L., and Stahl, R/F. 2023. “Complications of Cardiopulmonary Bypass from an Anesthesia Perspective: A Clinical Review,” Online, [Hospital Corporation of America] Healthcare Journal of Medicine 4(1): 13-21; Malil, S. and Haghaninejad, H. 2019. “Pulmonary Complications following Cardiac Surgery,” Online, Archives of Medical Science Atherosclerotic Diseases 4: e280-e285; Xu, S., Liu, J., Li, L., Wu, Z., Li, J., and 6 others 2019. “Cardiopulmonary Bypass Time is an Independent Risk Factor for Acute Kidney Injury in Emergent Thoracic Aortic Surgery: A Retrospective Cohort Study,” Online, [BioMed Central] Journal of Cardiothoracic Surgery (London, England) 14(1): 90; Murphy, G. J. an Angelini, G. D. 2004. “Side Effects of Cardiopulmonary Bypass: What is the Reality?,” Journal of Cardiac Surgery (Hoboken, New Jersey) 19(6): 481-488).

Inline coupling jacket-secured prostheses can be used to repair defects anywhere along the vascular tree in adults. Often these will be abdominal aortic aneurysms, whether congenital or acquired, caused by a weakening of the aortic wall due to atherosclerosis or an infection and secondarily aggravated by hypertension. Using the means described below in section 2b(2)(c)(c2), entitled Automatic Insertion into a Ductus of an Inline Coupling Jacket-secured Bridging, or Tie-line and Its Replacement if Necessary, the defect can be replaced, antibiotics or antifungals can be directly targeted to the prosthesis in greater concentration than would be concurrently administered systemically, and if present, local hypertension can be alleviated mechanically with a passive or ‘tuned’ shunt, as well as through the automatic direct pipe-targeting of antihypertensive and tissue strengthening medication.

Alternative methods of repair such as endovascular stents, stent grafts, patches, and hybrids of these can be displaced by the pulse and can dehisce and leak. Moreover, transluminal placement of an endoluminal stent-graft risks iatrogenic injury such as incisions or dissections which can result in a pseudoaneurysm and/or endoleak and if left untreated or undetected, can rupture. In pediatric patients, these are known not only not to expand with growth themselves but to prevent growth in the adjacent tissue resulting in impaired circulation and development.

These shortcomings of conventional measures for the treatment of aneurysms and/or coarctations for example are documented below with references to the literature in sections 2b(2)(d), entitled Anoxia-free Aortic Repair: Aortic Prostheses Thoracic, Thoracoabdominal, and Abdominal, and 2b(2)(h)(h1), entitled The ‘Tuned’ Shunt, and its subsections. Even though it would provide outcomes superior to conventional solid organ transplantation from every standpoint, the ability to replace an incompetent with a competent heart, for example, through a compound vascular bypass procedure with no interruption in the flow of blood, the need for cardioplegia, or general anesthesia would at first appear impossible; however, vascular servovalves make this possible.

The continuity of blood flow throughout a repair and the elimination of stitched junctions made possible by side-entry vascular valves, servovalves, and inline coupling jacket-secured aneurysm of other defect prostheses and bypass conduits should result in major improvements in the repair of congenital as well as acquired vascular defects. In a neonate, time to develop collateral circulation as in a patient born with a less pronounced congenital coarctation who was able to survive beyond the postnatal period is denied, making early repair of the defect imperative.

The spontaneous response of the body to develop collateral circulation in order to bypass an obstruction is life-sparing but due to a coarctation results in hypertrophic compensatory collateral arterial circulation expressed as [upper extremity] “arterial hypertension with a positive gradient between upper and lower limbs,” (Rossi, U. G., Ierardi, A. M., Carrafiello, G., and Cariati, M. 2020. “Aortic Coarctation,” Online, Aorta Journal (Stamford, Connecticut) 8(2): 46-47)(see also Law; M. A. and Tivakaran, V. S. 2023. “Coarctation of the Aorta,” Online, Treasure Island, Florida: StatPearls Publishing Co.; Panzer, J., Bove, T., Vandekerckhove, K., and De Wolf, D. 2022. “Hypertension after Coarctation Repair—A Systematic Review,” Online, Translational Pediatrics (Hong Kong, China) 11(2): 270-279; Vigneswaran, T. V., Sinha, M. D., Valverde, I., Simpson, J. M., and Charakida, M. 2018. “Hypertension in Coarctation of the Aorta: Challenges in Diagnosis in Children,” Pediatric Cardiology (New York, New York) 39(1): 1-10; Convens, C., Vermeersch, P., Paelinck, B., Van den Heuvel, P., and Van den Branden, F. 1996. “Aortic Coarctation: A Rare and Unexpected Cause of Secondary Arterial Hypertension in the Elderly,” Catheterization and Cardiovascular Diagnosis (New York, New York) 39(1): 71-74; Engvall, J., Karlsson, M., Ask, P., Loyd, D, Nylander E., and Wranne, B. 1994. “Importance of Collateral Vessels in Aortic Coarctation: Computer Simulation at Rest and Exercise Using Transmission Line Elements,” Medical and Biological Engineering and Computing 32(4 Supplement): S115-S122). Unlike collateral circulation that develops to compensate for an obstruction in a coronary artery, here the adaptation does not institute normal circulation.

Provided the coarctation is narrow, the best conventional repair is open surgery to excise the defect and directly end-to-end anastomose the cut edges. Successful repair thus reduces the odds, high with congenital coarctation, of recurrence and will usually eliminate the need for revision surgery (see, for example, Agasthi, P., Pujari, S. H., Tseng, A., Graziano, J. N., Marcotte, F. and 4 others 2020, Op cit. above in Section 2. Background of the Invention. a. General; Wilson, W., Osten, M., Benson, L., and Horlick, E. 2014. “Evolving Trends in Interventional Cardiology: Endovascular Options for Congenital Disease in Adults,” Canadian Journal of Cardiology 30(1): 75-86). With a more extended coarctation, especially if adjacent tissue is malformed or aneurysmal, the thoracic aorta can be replaced with a prosthesis of the kind described herein. In a child, this should be considered essential.

When the aortic wall is tenuous and likely to aneurysm, a seamless inline coupling jacket-secured expandably structured thoracic aorta prosthesis as described below in section 2b(2)(f), entitled Expandable Structure to Accommodate Growth and Self-adjust in Size in Vascular Segment Prostheses, Lines, and Confluence Conduits Placed in Children, is suitable for more extended defects such as a coarctation or interruption. When these conditions do not pertain, smaller defects can either be conventionally excised with the stumps end-to-end anastomosed, or a side-entry diversion jacket-secured bypass placed. The bypassed tissue should atrophy. Coarctation effectively a less extensive expression of the same underlying cause as more extensive complete, not just constricted, interruptions in the aortic arch, balloon angioplasty without follow-up endoluminal stenting can yield adverse sequelae, such as dissections, pseudoaneurysm, or re-coarctation.

Apart from dissection, angioplasty can stretch the vessel wall to result in quick restenosis, dissection, and/or facilitate the development of an aneurysm, resulting in a consequence no less problematic than was that primary, to include recoarctation, prompt stenosis, and aneurysm (see, for example, Law; M. A. and Tivakaran, V. S. 2023, Op cit., above in this section; Suradi, H. and Hijazi, Z. M. 2015. “Current Management of Coarctation of the Aorta,” Online, Global Cardiology Science and Practice (Doha, Qatar) 2015(4): 44; Chiu, H. H., Wang, J. K., Chen, Y. S., Chiu, I. S., Chang, C. I., and 5 others 2013. “Long-term Outcomes of Native Coarctation of the Aorta after Balloon Angioplasty or Surgical Aortoplasty in Newboms and Young Infants Less Than 3 Months of Age,” Online, Acta Cardiologica Sinica [Zhonghua Minguo Xin Zang Xue Hui Za Zhi] 29(2): 168-174).

The need for a perpendicularly protrusive side stem indissociable from valving between either of two outlet passages from the same native lumen, encroachment upon neighboring tissue can almost always he avoided by rotating the valve and/or shifting in level along the inlet line, both adjustments without restriction. Where alternative outlet paths are provided, the ability to continuously meter the relative proportion of flow volume between the two affords significant advantages. For example, a sensor-detectable mishap of any kind affecting either outlet pathway can be automatically responded to by the implanted disorder response system by switching flow into the alternative path. As shown in FIG. 22B, for example, flow can be diverted from the anastomotic through the bypass or the reverse an any relative proportion with no interruption in the functioning of the tissue or organ supplied.

In most cases, the midprocedural bypass will itself become the replacement prosthesis, with the inlet diversion jackets positioned upstream and the outlet jackets positioned downstream. For this reason, in a young patient whose growth makes eventual replacement certain, a segment that will provide sufficient space toward the aortic root should be furnished with the expandable structure delineated below in section 2b(2)(f), entitled Expandable Structure to Accommodate Growth and Self-adjust in Size in Vascular Segment Prostheses, Lines, and Confluence Conduits Placed in Children. If and only if unavoidable, replacement once again consists of connection of the diversion jackets as straddling, or superimposed over those original long since left to heal.

The references cited below in section 2b(2)(f), entitled Expandable Structure to Accommodate Growth and Self-adjust in Size in Vascular Segment Prostheses, Lines, and Confluence Conduits Placed in Children, and generalizing, the increase in the external diameter of the thoracic aorta at the arch due to growth from neonate to adulthood across races and sexes, is roughly from 0.8 or 1.0 to 2.5 or 3.0 centimeters which can continue increase in old age. Exactitude is hampered due to the different imaging and measuring methods used (Elefteriades, J. A., Mukherjee, S. K., and Mojibian, H. 2020. “Discrepancies in Measurement of the Thoracic Aorta,” Online, Journal of the American College of Cardiology 76(2): 201-217). For this reason, the expandable structure addressed below in section 2b(2)(f) is made to cover the range of sizes specified.

### Alternative Method for Placing an Incline Coupling Jacket-Secured Tie-Line Type Vascular Prosthesis

The side-entry and incline coupling devices described herein provide three methods for inserting a prosthetic segment along a great or major vessel without interrupting the flow of blood, hence without anoxia or the need for cardiopulmonary bypass or general anesthesia. One method is addressed above in section 2b(2)(c), entitled Vascular Segment Prostheses: Inline Coupling Jackets and Bridging, or Tie-lines, and its subsections. A second method for use in an adult is the temporary use of a side-entry jacket-secured bypass across the segment to be resected short of the stumps to remain for stitching to a woven poly(ethylene terephthalate) polyester Dacron® patch to bridge between stumps.

Also insertable thus would be an improved tissue engineered neoarch, for example, or one immediately harvested from a deceased donor having been kept on life support prior to and continued after death. The third method is to use a side-entry jacket-secured bypass across the segment to be resected short of the stumps to remain for manual attachment of an inline coupling jacket-secured prosthesis without resorting to the automated insertion capability. In a child the tie-line is made using the expandable structure.

This last method allows the use of an inline jacket-secured tie-line type prosthesis to be placed where for any reason, the affected or substrate vessel is configured as to thwart its substantially linear conformation required for use of the automated placement process described below in section 2b(2)(c)(c2), entitled Automatic Insertion into a Ductus of an Inline Coupling Jacket-secured Bridging, or Tie-line and Its Replacement if Necessary. While the operator or an assistant can manually adjust the device to accommodate a minor deviation from the normal, the automated process is ultimately mindless. A seamless leak and migration-proof inline jacket-secured tie-line type prosthesis with at least one drugline for the direct targeting of medication is to be preferred to alternative forms of repair, all of which have a propensity for requiring painful reentry for repair or replacement at a later date.

This third, or manual method should be performed under the watchful monitoring of a pacemaker-cardioverter/defibrillator which can be removed once the tie-line has been fixed in position. Because manipulation of the aorta can induce complications elsewhere in the arterial tree, whenever some anomaly of the aorta does not require manual placement, manipulation should be kept to the minimum by using the automated process (see, for example, Anomalies of the aortic arch and their repair can provoke seemingly distant complications.

Handling the aorta can provoke serious complications (see, for example, Bordes, S., Vefali, B., Montorfano, L., Bongiomo, P., and Grove, M. 2023. “Evaluation and Management of Complications of Endovascular Aneurysm Repair of the Thoracic Aorta,” Cureus (Palo Alto, California) 15(3): e36930; Faiza, Z. and Sharman, T. 2023. “Thoracic Aorta Aneurysm,” Online, Treasure Island, Florida: StatPearls Publishing Co.; Yun, Y. I., Chang, C. H., Kim, J. H., and Jung, Y. J. 2023. Treatment for Subarachnoid Hemorrhage Due to Ruptured Posterior Cerebral Arterial Dolichoectasia with Aortic Arch Anomaly,” Online, Journal of Cerebrovascular and Endovascular Neurosurgery (Seoul, South Korea) 25(1): 69-74; Mantri, S. S., Raju, B., Jumnah, F., Rallo, M. S., Nagaraj, A., and 5 others 2022. “Aortic Arch Anomalies, Embryology and Their relevance in Neuro-interventional Surgery and Stroke: A Review,” Online, Interventional Neuroradiology (Thousand Oaks, California) 28(4): 489-498; Zachary Chadnick and Kuldeep Singh 2021. “Complications Associated with Aortic Aneurysm Repair,” Online, Intech Open Aortic Aneurysm, in Guillaumon, A. T. and Siquira, D. (eds.), Aortic Aneurysm: Clinical Findings, Diagnostic, Treatment and Special Situations, Intech Open: Rijeka, Croatia; Hanneman, K., Newman, B., and Chan, F. 2017. “Congenital Variants and Anomalies of the Aortic Arch,” Radiographies (Oak Brook, Illinois) 37(1): 32-51; Yamasaki, K., Yokogami, K., Ohta, H., Yamashita, S., Kawasoe, T., and Takeshima, H. 2015. “Agenesis of the Left Internal Carotid Artery in the Right Aortic Arch with Isolation of the Left Innominate Artery Associated with Ruptured Cerebral Aneurysm: Case Report,” Online, NMC [Neurologia Medico-Chirurgica/Japan Neurosurgical Society] Case Report Journal 2(2): 46-48; additional references cited above in section 2a, entitled Background of the Invention. General).

The risks in replacing a growth-unresponsive repair in the thoracic aorta to accommodate the growth having occurred since can be reduced through the use of a midprocedural bypass which continues blood flow throughout the procedure where the use of the expandable structure tie-line type prosthesis described in this and its subsidiary sections would have averted the need for a painful revision in the first place. Also, the defective tissue if bypassed during the initial procedure, there may not be adequate space to the sides to place a bypass. However, incorporating at least one accessory channel for the insertion of a drugline, the expandable prosthesis would have little need for a peri- or postprocedural bypass which would invite the risk of unnecessary complications.

The compact adjacency of the great vessels severely limits conventional side-entry with the outflow diverted normal, or perpendicularly, to the substrate ductus, such as used for the ureteral takeoffs shown in FIGS. 28 and 30. If used, jackets should have a pliant, angled, sidestem 19 and be relatively thin. Especially if to be retained after closure, diverted flow would best parallel to approximate the normal path with minimal outward projection of a side stem near to the aortic root as would protrude into the main pulmonary artery or trunk, for example, to interfere with its streamline flow in proportion to the degree of intrusion.

In these regards, an aneurysm, coarctation, or interrupted aortic arch bypass conduit takes a form different than the diversion jackets and valves used elsewhere in the vascular tree or in the lower urinary tract such as those shown in FIGS. 17B, 22A and 22B, and 22F, for example. For any implant, the minimization of obtrusiveness where there is the likelihood of discomfort for the patient due to the use of components excessive in size and weight is the prime prerequisite. The aortic arch poses the most severe conditions in the circulatory system.

This includes unremitting pulsation of blood close to the aortic valve, hence, proximity to the site of maximum cardiac ejection pressure from the left ventricle under pulsatile peaking pressure, and proximity to the carotid arteries with the risk of embolizing debris causing a stroke. At a site where rupture or obstruction is a medical emergency, the frequent onrush of blood at high pressure could not be better designed to damage or wear out an implant placed in its path. Means to minimize extravasation during placement of the inlet and outlet diversion jackets seek to preclude fouling of the jacket linings by even a minor seepage.

With a prosthetic disorder response system, where switching between either (or in some cases, both) of two pathways of the blood ordinarily passed through a single vessel-one native, the other prosthetic—in response to sensor input can be applied, switching between the native and synthetic lines are made automatic to proceed instantly, possibly averting the risk of sudden death. Bistable, or either/or pathway switching of flow is with a solenoid-driven side-entry vascular valve, while apportionment between the paths requires a side-entry vascular servovalve.

Where fibrous encapsulation of a sensor that would interfere with signaling can be avoided, conventional sensors to detect medically significant changes in blood pressure, pulsation, and oxygenation, for example, can be incorporated inside the flow diversion valve jackets. A tie-line type prosthesis can also incorporate sensors. In either, the sensors signal the implanted microcontroller, or in comorbid disease, the master control microprocessor, to extend or retract the valve diversion chute.

An adjustable sensor-responsive servovalve can dynamically adapt to the instant blood pressure by tapping off and passing a portion of the flow volume to another large vessel such as the superior vena cava. An example of such an arrangement is described above in section 2b(2)(h)(h1), entitled The ‘Tuned’ Shunt, and its subsections and shown in FIG. 22F. In an aneurysm or coarctation bypass conduit for a patient with essential or secondary hypertension but otherwise normal vascular tissue, a fixed flow-rate side-entry diversion servovalve may precede or be integrated into the bypass inlet diversion jacket.

Upstream elevation of the pressure in the pulmonary arteries is generally due to an upstream impedance posed by pulmonary vascular resistance or an obstruction such as a stenosis, whereas a downstream elevation that affects the great arteries is due to a primary and/or secondary systemic hypertension, which affects all the arteries. Not curative, these electromechanical means for achieving pressure relief are expedients to prevent injury to tissue where the primary object should be to effect a cure whether surgically through a lung or liver transplant, for example, or medicinally.

Vascular defect bypass conduits are intended to supplant the use of woven edge-stitched nitinol-reinforced polyethylene terephthalate (Dacron®, Terylene®) or reinforced Gore-Tex® patches and stent-grafts to repair aortic arch aneurysms and coarctations, for example. This is especially true with respect to the repair of such defects in infants, who often cyanotic, are identified promptly following birth, hence, those most often treated thus.

The touted high tensile strength of these patches may be appropriate in an adult but fundamentally harmful in a pediatric patient. In a child, not just the patch itself but the structure surrounding it and often the left arm is restricted from normal growth. Aneurysm or coarctation bypass conduits do not include a pressure relief shunt or ‘tuned’ shunt as shown in FIG. 22F to tap off blood from the thoracic aorta to the pulmonary trunk, or main pulmonary artery, for example, as would mix oxygenated and unoxygenated blood adulterating both.

Continuous adjustment to elevated blood pressure in the pulmonary trunk can be achieved with a servovalved shunt to tap off a sufficient volume of blood flow to reduce the pressure. A nonjacketing side-entry connector as described in U.S. Pat. No. 11,013,858, entitled Nonjacketing Side-entry Connectors and Prosthetic Disorder Response Systems, connected to the pulmonary trunk and/or aorta can be used to separately pipeline target such medication to either or both sites.

An inlet inline coupling jacket-secured neoarch or a servovalve inlet-secured replacement aortic arch incorporates at least one accessory channel for the insertion of a drugline to release medication into either type device. Comparably, in pulmonary or portal hypertension where the vessel is to remain intact or be provided with a servovalve-secured tap-off or ‘tuned’ shunt, a side-entry flow diversion jacket can be used to release a vasodilatory topical antihypertensive such as nitric oxide-releasing nitroglycerin or nitroprusside.

That systemic antihypertensives such as thiazide diuretics, angiotensin converting enzyme inhibitors, angiotensin II receptor antagonists, and calcium channel blockers can be centrally targeted directly into the aortic arch and/or pulmonary trunk for systemic and pulmonary dispersal through accessory channels in the associated side-entry jacket or through a proximate nonjacketing side-entry connector and if requiring liver passage is processed in post-liver form for topical use is considered obvious.

If intercurrent, systemic hypertension can be treated conventionally with an oral antihypertensive as secondary or adjunctive to that topical as primary. Otherwise, tuned shunts are addressed below in 2b(2)(h)(h1), entitled The ‘tuned’ shunt, and its subsection, while portal hypertension is addressed below in section 2b(2)(h)(h2), entitled The ‘Tuned’ Shunt in the Relief of Portal Venous Hypertension, and pulmonary hypertension below in section 2b(2)(h)(h3), entitled The ‘Tuned’ Shunt in the Relief of Pulmonary Arterial Hypertension, an exemplary tuned shunt shown in FIG. 22F.

2b(2)(c)(c1). Inline Coupling Jackets Proper

A structural description of inline coupling jackets is provided below in section 6, entitled Description of the Preferred Embodiments of the Invention. Referring to FIG. 37A, the outer half-jackets clutch about the stumps of the native vessel and must incorporate features to dispel atherogenic degeneration induced through compression of the vasa vasorum and nervorum as addressed with references to the literature in U.S. Pat. No. 11,389,171, entitled Integrated System for the Infixion and Retrieval of Implants. Such features include numerous apertures and the use of parylene-coated viscoelastic polyurethane foam which thermoplastic, invests rather than compresses the vasa vasorum and vasa nervorum.

Additional antiatherogenic measures consist of the automatic release of protective substances by an implanted disorder response system described in U.S. Pat. No. 11,759,186, entitled Ductus Side-entry Jackets and Prosthetic Disorder Response Systems, and copending application Ser. No. 17/689,880, entitled Prosthetic Disorder Response Systems. For use in neonates and infants likely to grow to a large size, to provide sufficient expansion to adulthood, eight axially oriented expansion joints are positioned equidistantly about the circumference of the outer half-jacket outer housings. Only the outer half-jackets which encircle the native stumps require through-and-through apertures to dispel a

Inline coupling jackets are not only entry and exit end-connectors used to form durable junctions between native stumps and interpose tie-lines or tie-line type prostheses to replace a segment along a native ductus, but are also the means by which the replacement segment is fixed in position. Unlike end-insertion line connectors such as those shown in FIGS. 31A thru 31C, inline coupling jackets allow for growth and can be used in children of any age to eliminate the need for later reentry. Usually applicable to ductus with little surrounding clearance, inline coupling jackets can be made smaller in diameter than as appears in the drawing figures; however, to allow for growth, the difference must be less than possible otherwise.

As shown in FIGS. 22C thru 22E, vascular servovalves can be used to bypass or ‘jump’ around and apportion a continuously variable fraction of the total flow through a parallel segment along a ductus, usually a vessel, into another vessel. This requires an adjustable bypass entry diversion chute, which extending laterally, even when angled, disallows a more in-line shape. An alternative method for obtaining continuous adjustability in flow, a motorized iris valve—is bulky, poorly adapted for implantation, and does not tap off a fraction of the flow for passage into another ductus as does a vascular servovalve as is often necessary in surgical procedures as well as in end-constructions such as a localized vascular hypertension ameliorated by a continuously variable pressure sensor responsive shunt.

Rather, like the servochoke type valve shown in FIG. 14A, it variably reduces and enlarges the flow through cross-sectional area. Inline coupling jackets and end-insertion line connectors are applicable where no continuously variable portion of flow for diversion into another vessel is involved and the object is rather to provide an in-line connector between successive segments with as little lateral protrusion into neighboring tissue as possible. Minor alterations in conformation such as angling notwithstanding, where continuously variable control over the apportionment of flow between two outlets is required, a diversion chute extending and retracting through a laterally extended side-stem is needed.

A jointed diversion chute that would deploy from a sleeve parallel to the ductus lumen through a guideway would still require a protrusive servomotor. Accordingly, the appearance of diversion chutes, part number 18 as in FIGS. 17B and 22C thru 22F, for example, denote an application wherein the controllable division of flow between either of two outlet passages is present. In applications involving unidirectional flow, it may be possible and a cost saving measure to use a servovalve at the entry into a mainline, or bloodline, and a ‘dumb’ or simple flow diversion side-entry jackets as described below in section 2b, entitled Vascular Valves and Servovalves in the Diversion of Blood Flow, in the subsection thereof entitled Simple Flow Diversion Side-entry Jackets, shown in FIGS. 10F and 10G at the outlet. Both the servovalve and the simple diversion jacket incorporate and would be indicated in the drawing figures as a diversion chute, the description in the text making it clear which type side-entry device shown in the drawing figure is meant.

While conventional means of repair along the thoracic aorta, for example, too often fail with regard to dislodgement or migration, these prostheses must not accommodate the growth of a pediatric patient, but must remain in place with zero migration over a period of many years, or even for life. The terms used to specify the different surfaces of inline coupling jackets used to secure either end of the prosthesis to the stumps of the native ductus are defined below in this section.

The first part through which blood enters is the inlet half of the entry jacket, which encircles to grasp about the cut end of the entry vessel stump, while and outlet half of the entry jacket encircles the upstream end of the tie-line. The exit jacket encircles to grasp about the other end of the tie-line, the outlet half of the exit jacket encircling or connecting the prosthesis to the upstream stump of the substrate vessel or ureter. The stitched method for fixing the jackets in place shown in FIG. 37D requires that the outer half-jackets be solid and securely connected to the native stump at either end. Solid, the outer half-jackets to not grow in length, Instead, the jackets accommodate growth through the gradual increase in compression of their foam lining when the stumps expand in diameter.

This allows the stumps, and in the inner half-jackets, the lumina of the prosthesis, to expand. As shown in FIG. 37D, fixation of the outer inline coupling half jackets used to securely connect the prosthesis to the stumps of the substrate vessel at either end is with through-and-through sutures 232. Radially oriented, the sutures do not interfere with the longitudinal expansion of the outer half jackets in step with the growth of the stumps. The increase in growth ordinarily less, a need to cover the difference in caliber all the way from birth to adulthood in a large male is seldom necessary. For a neonate whose parents are above average in size, inline coupling jackets for placement along any given vessel may additionally incorporate stretchable expansion joints. These should be spaced equidistantly about the circumference of each paired inline coupling half-jacket.

Calibers of the thoracic and abdominal aorta where congenital defects more often appear have been widely studied (see, for example, Oluwole, O. O. 2023. “Reference Values for Paediatric Abdominal Aorta on B-mode Ultrasound—An Outer-to-outer Methodology,” Pediatric Radiology (Berlin, Germany) 53(5): 875-884; Kale, S. V. and Alam, S. 2020. “Determining the Normal Effective Diameter of Thoracic Aorta in Pediatric Population of India,” Online, Indian Journal of Radiology and Imaging (Stuttgart, Germany) 30(2): 170-176; Akturk, Y. and Ozbal Gunes, S. 2018. “Normal Abdominal Aorta Diameter in Infants, Children and Adolescents,” Pediatrics International (Carlton Melbourne Victoria Australia) 60(5): 455-460; Hegde, S. V., Lensing, S. Y., and Greenberg, S. B. 2015. “Determining the Normal Aorta Size in Children,” Radiology (Easton, Pennsylvania) 274(3): 859-865).

Size ranges for other vessels from neonate to adult is a subject too voluminous to be covered here. Suffice it to say, inline coupling jackets can be made with a foam lining thick enough to be compressed to accommodate a large increase in the diameter of the encircled vessel. Exceptionally, it may be desired to stabilize the inner half-jackets clutching the stumps of the substrate aorta. In that case, referring to FIG. 37D, drilling discouraged as generating potentially embolizing debris, to pass suture through the hard polymeric outer casing of either inner half-jacket, pass-through holes can be created with a needle heated over the melding point of the polymer. Moreover, the use of a hot needle produces a hole somewhat larger than needed to provide clearance for the suture to pass freely through the holes without abrasive resistance.

Suture passed entirely through the half-jacket restrains the native vessel from elongating, so that the pressure associated with growth is accommodated by expanding the vessel diametrically, and since the suture passes through diametrically, the suture does not restrain enlargement in diameter. Depending upon the size range to be covered, inline coupling jackets for placement in a fetus or neonate may need to incorporate stretchable expansion joints. The inner half-jackets encircle the stump-ward ends of the expansion structure within the foam lining and outer hard polymeric casing.

As shown in FIG. 37D, atherosclerotic-counteractive and suture holes 40 in the outer half-jackets which cinch about the stumps of the substrate vessel are through the outer casing with expansion to accommodate growth restrained in the long central axis but not diametrically. Moreover, in a growing child, as the pressure within the half-jackets increases due to growth, the native stumps in the outer half-jackets accommodate the need for an increase in internal diameter by compressing the atherosclerosis protective and dissolution counteractant-coated compliant viscoelastic polyurethane foam lining with through-and-through apertures 40.

These measures serve to protect the fine nervelets and vessels at the adventitial surface from compressive injury and the provocation of an atherogenic (atherogeneic) reaction that otherwise would lead to atheromatous degradation. In contrast, the inner half jackets clinching the ends of the prosthesis accommodate growth due to their expansion structure as delineated shortly above in section 2b(2)(f), entitled Expandable Structure to Accommodate Growth and Self-adjust in Size in Vascular Segment Prostheses, Lines, and Confluence Conduits Placed in Children. Only where the extent of growth is anticipated to be considerable are axially oriented stretchable expansion joints added.

The expansion structure in the inner half-jackets which encircle the ends of the inanimate polymeric prosthesis need no such apertures. The walls of the prosthesis likewise pass over the suture through the suture holes freely, allowing both outer and inner half-jackets to expand to much the same diameter together as their inner to outer half jacket junctions depicted in FIGS. 36C and 37C. The outer inline coupling half-jackets encircle the substrate vessel up to the distance to the miniature serrated reciprocating saw part number 226 in FIGS. 37E and 37F that will transect the vessel. As shown in FIG. 37A, for example, the downstream inline coupling half-jackets the same but positioned in reverse.

For repairs of vascular vessels which cannot be accomplished by short resection and end-to-end anastomosis, that the use of a synthetic prosthesis is unsusceptible to disease and does not require the harvesting of autologous or allogeneic tissue as a separate operation with many possible complications is a distinct advantage. A good patch properly used offers the same advantages but not in a small pediatric patient.

A bloodline connected at either end with side-entry diversion jackets, valves, servovalves, or a servovalve at the entry end and a passive flow diversion jacket at the outflow end can be used to span across a defect and thus bypass an aneurysmal, stenosed, or otherwise defective or diseased segment along a major vessel. The benefit in using these devices is that they eliminate the need to interrupt the flow of blood through the substrate vessel.

If the laterally extended sidestems of controllable side-entry devices, part number 19 in the drawing figures, would protrude into neighboring tissue and gaining controllability over the rate of flow through the substrate vessel is not a factor, an inline coupling jacket-secured tie-line prosthesis can be inserted to replace the affected segment, again, without interrupting the flow of blood through the substrate ductus. Also, as shown in FIGS. 22A, 22B, and 32D thru 32F, switchable, valves and servovalves allow switching between the native and synthetic routes, of which the potential advantages include diversion to allow healing, for example.

Side-entry servovalves allow continuously variable control over the volume of blood flow through which inline coupling jackets, not valves, cannot. If the patient is a child, the tie-line of anatomically simulating prosthesis secured at either end is made as described below in section 2b(2)(f), entitled Expandable Structure to Accommodate Growth and Self-adjust in Size in Vascular Segment Prostheses, Lines, and Confluence Conduits Placed in Children, which will expand both in length and diameter.

Otherwise, these are used without the interposition of a confluence conduit to connect a tie-line, or segment-bridging line connected by an inline coupling jacket at either end. The bridging, tie-line, or prosthesis joining the jackets is meant to replace a diseased, malformed, or injured native segment, usually along a vessel or ureter. Such a tie- or bridging line elaborated to incorporate branches constitutes a prosthesis for replacing a larger vessel.

FIG. 36E assigns part numbers to the entry and exit surfaces of each half-jacket, Inline coupling jackets consist of two side-by-side half-cylindrical sections, each comprising an outer soft, highly elastic, and compliant, long chain covalent cross-linked vulcanized viscoelastomeric casing with a core mesh of biaxially-oriented stretched polyethylene terephthalate polyester film to impart an end-tensile strength and elastic or tensile limit, that is, the rupture or bursting force, sufficient to withstand a strong direct blow, underlain by a viscoelastic foam at the center of which is the cylindrical hollow, or vacuity in which the native ductus is held, that is, into which the native segment will remain until the rotation of the other jacket halves, rotated about to emplace the tie-line or prosthetic replacement segment, are closed to transect and displace the native ductus.

Provided the edges of the stumps and the prosthesis are well apposite, the complete enclosure within the jackets will form a continuous passage to avert bleeding. An aortic neoarch is one example, an abdominal aorta segment replacing prosthesis another. A type connector which maintains strong adhesion to its substrate even while expanding and contracting in response to changes in the pressure of fluid passing through is without precedent. Delineated in this section and in Section 6 below, entitled Description of the Preferred Embodiments of the Invention, under the subsection thereof entitled Artificial Aorta and Its Automatic Placement is the construction of inline coupling jackets to allow their expansion and contraction in step with changes in pressure passing through the ductus.

Prostheses for vessels, or bloodlines, segments or uniform diameter along the abdominal aorta, for example, and along druglines are symmetrical while those for an entire thoracic aorta take into account its gradual reduction in diameter in the plunge down into the abdomen. Unlike the outlet half of the entry jacket and the inlet half of the exit jacket, which clutch about either end of the tie-line (bridge, anatomy replacing, or central) portion of the prosthesis, the inlet half of the entry jacket and outlet half of the exit jacket clutch about the stumps of the substrate native vessel to either side of the prosthesis and therefore incorporate the protective components essential to avert the atherosclerotic degeneration that would otherwise affect a vessel if fully enclosed. Delineated in U.S. Pat. No. 11,759,186, entitled Ductus Side-entry Jackets and Prosthetic Disorder Response Systems, vascular valves are side-entry jackets modified to incorporate a diversion chute.

Likewise enwrapping a native ductus, vascular valves incorporate apertures entirely through the jacket giving exposure of the adventitia to the surrounding space, a highly compliant investiture which does not compress the vasa vasorum and vasa nervorum as a jacket lining of viscoelastic polyurethane foam barrier-coated with vapor deposited elastic parylene, for example, to prevent the release of 2,4-toluenediamine, for example, and means for directly pipe-targeting medication such as anti-inflammatory to the adventitia. Enclosing the ends of the synthetic tie-line, these measures are unnecessary in the outlet half of the entry jacket and the inlet half of the exit jacket or jackets. Section 2a above, entitled Background of the Invention. General, dealt with this in more detail.

The outer shell of the inlet half of the entry jacket and outlet half of the exit jacket can made of any implantable polymer such as polyether ether ketone, nylon, or a fluoropolymer with apertures 40 to minimize compression of vasa vasorum and nervorum which also serve as the holes to pass through suture at 90-degree intervals about the circumference in order to fix the prosthesis in position. Necessarily constrained in length, the growth of the stumps is accommodated by expansion in diameter, by the investing but highly compliant parylene vapor deposition coated viscoelastic foam.

Whether for use in an adult or a child, elasticity sufficient to comply with the pulse is essential in any embodiment. Securing the ends of the synthetic tie-line, the outlet half of the entry inline coupling jacket and inlet half of the exit jacket in a pediatric embodiment require taking no additional measures to protect enclosed native vessel stumps which must still comply with the pulse and in a child, accommodate growth.

As with the outer native vessel stump-clasping half-jackets, expandability in the inner prosthesis or tie-line clasping half-jackets is not by incorporating the flats 233 and vaults 234 structure depicted in the drawing figures but passively by the increase in internal diameter response to the increase in pressure associated with growth. As to the prosthesis or tie-line proper, the expandable structure described below in section 2b(2)(f), entitled Expandable Structure to Accommodate Growth and Self-adjust in Size in Vascular Segment Prostheses, Lines, and Confluence Conduits Placed in Children, accommodates growth as well as the pulse and spontaneous adjustments in vessel caliber such as due to posture and intense exertion by the autonomic system.

The terms used to specify the relative positions of inline coupling jackets along the prosthesis as a whole are defined above in section 2b(2)(c)(c1), entitled Inline Coupling Jackets Proper. FIG. 36A shows a schematic side view of tie-line with inline coupling jackets to secure it in position. Delimited defects such as less extensive aneurysms, dissections, coarctations, and interrupted aortic arch replacement prostheses are generally tubular, while more extensive prostheses such as an aortic neoarch with supraaortic branches as shown in FIG. 36F are anatomical, and expandable to allow for growth in an infant, need not be made in different sizes. The entry jacket to the left and exit jacket to the right of the connecting tie-line at either end are shown in transverse longitudinal axis section. The exit jacket is identical to the entry jacket but reversed in position as the antipode, or mirror image, of the entry jacket.

The exit jacket likewise comprises two half cylinder parts separated by the transection mechanism shown in FIG. 36C, of which the half on the inflow side encircles and retains the aortic stumps as shown in FIG. 37D secured in position by through-and-through sutures placed through the atherogenesis counteractive apertures of the outer half jackets 40, made easy due to the translucence if not transparency of the polypropylene or polyetheretherketone jacket while that on the outflow side encircles and is inseparably cinched about the polymeric aortic neoarch.

The outlet jacket has the same structure but is reversed along the axis of the conduit so that as shown, the polymeric aortic arch replacement conduit is inseparably fused to its inlet, as shown, left half and the aorta resumes as grasped within its right half. The halves of the jackets are hinged at the circumference to rotate separately during placement and together with both closed to fix the jacket in position. The inlet half of the jacket is closed and locked about the aorta with a diametrically positioned catch at the jacket circumference opposite the hinges.

That the fabric, the internal construction of inline coupling jackets, and the mechanism which makes it possible for an aortic prosthesis using these components to self-install (under the watchful eyes of the operator and an assistant) are subjects intimately interconnected and overlapping. For these reasons, reference to automatic insertion appears in several sections, to include the opening section entitled Background of the Invention and that entitled Artificial Aorta and Its Automatic Placement, as well as this section.

The construction of inline coupling jackets which allows these to expand and contract in step with changes in pressure passing through the ductus while holding the substrate in position tenaciously until growth necessitates replacement if is delineated above in this section and below in the section 6, entitled Description of the Preferred Embodiments of the Invention, in the subsection thereof entitled Artificial Aorta and Its Automatic Placement. The resultant structure can be tested by passing through pulsating water for an expansion-contraction profile simulating that of a large artery with elastic laminae. If necessary, the polymers are reformulated by adjustment in the incorporation of plasticizers or copolymers until this action is satisfactory.

To avert migration or disengagement, the ends of the replacement segment, or tie-line type prosthesis, are attached to the retaining inlet half of the prosthesis entry and outlet half of the exit jackets, that is, the inner half-jackets indirectly, by means of through-and-through stitching of the outer half jackets to the stumps of the substrate vessel at right angles. If felt necessary, the inner half jackets can be sutured likewise by passing a hot needle through the polymeric jacket.

As shown in FIG. 37D, a needle with clot repellent high tensile strength and clot-repellent suture is passed entirely through outer half-jackets, is allowed some slack, then reintroduced and passed entirely through these half-jackets at an angle of 90 degrees in relation to the initial pass, and so on. Protected by the thoracic outlet and sternum, the odds that a replacement thoracic aorta would be punctured or otherwise breached are no more likely than for such an injury with a native aorta. Conventional surgical repairs do not cover the aorta with a steel plate, for example.

The automated process of vascular prosthesis insertion immediately succeeding removal of the native segment in a single action is described above in section 2a. Where neither contact and pressure compliance nor the ability to expand with growth are essential, a requirement for native ductus-to-synthetic tubing connection or the reverse can be satisfied by end-inserted connectors such as those shown in FIGS. 31A thru 31C. These accomplish end-to-end connection without an increase in external diameter. Because inline coupling jackets will yield to external pressure, the use of these to encircle a native vessel coursing through a skeletal muscle would not, as would ordinary side-entry connectors, result in protrusive, abrasive, erosive, and painful sideways encroachment without the need to excise some of the adjacent muscle tissue.

However, unless negligible, the compliance of most (but not all) inline coupling jackets could result in compression of the substrate vessel, making end-inserted connectors the best choice where brief clamping is permissible. Clamping along the supra-aortic branches or coronaries is impermissible. Functionally the same, inline coupling jackets differ only in diameter to fit ductus different in caliber and in the incorporation or not of materials that lend a high degree of compressibility and expansion to accommodate growth, a layer of foam surrounding the native ductus still complying with the pulse and changes in blood pressure.

Inline coupling jackets only exhibit a switching function during replacement of a native with a prosthetic vessel, during which they avoid the sideways protrusion of side-entry devices which-unless rotatable or shiftable in level along the substrate vessel to a compatible disposition—can disqualify their use in compact anatomy where a lack of clearance would result in irritating contact with neighboring tissue. In contrast, the substantially linear conformation of inline coupling jackets allows placement to replace arterial or venous aneurysms in confined quarters, especially in the chest.

Thus, in tight anatomy such as in the chest, encroachment upon the great vessels is avoided. This allows the use of inline coupling jackets where the placement of a vascular prothesis with the aid of side-entry valves amid most viscera could not be tolerated. Both ductus side-entry and inline coupling jackets can incorporate one or more accessory channels to allow the direct pipeline targeting of drugs into the ductus connected and maintenance agents for the jacket itself through druglines. Always positioned along a single ductus, inline coupling jackets allow the replacement of extensive vascular segments in the tightest of quarters but never used in a switched organ, or compound vascular bypass organ transplant, need no ability to switch between either of two outlet passages.

A need for side-entry is associated with inter-rather than intracorporeal flow with a need for interorgan switching-ability. Critically, for numerous procedures in vascular surgery, bridging, or tie-line positioning along a vessel to replace the native segment is automated and without interruption in the flow of blood, which factor, otherwise unavoidable, is largely responsible for numerous midprocedural complications and postoperative adverse sequelae. Placement without clamping is the same consideration that prompted the design of the other ductus and tissue connectors specified above in the section entitled Cross Reference to Related Applications.

The blood or urine bridging tie-line, or conduit, connecting the entry to the exit jacket, can have the same conformation as the original ductus had before it had been affected, to include any branches it gave off. As shown in FIG. 36G, a bridging or tie-line can also be used as a mounting for a fixed diversion jacket or side-entry valve or servovalve, for example. Eventually, tissue engineered vessels with endothelial function and ureters with urothelial function, perhaps even peristalsis, should replace synthetic tie-lines.

Until then, one or more accessory channels leading into the tie-line entry jacket can be used to deliver drugs that simulate function of normal secretions produced by an epithelial lining. Inline coupling jackets per se are singular in inlet and outlet flow and therefore incapable of switching flow between either of two outlet lines as can solenoid-driven valves directly connected to the ductus, much less apportioning flow between either line as can servomotor-driven valves directly connected to the ductus. However, these switch-capable devices can be mounted along a bridging, or tie-line, connected at either end to the cut ends, or stumps, of the substrate native ductus by inline coupling jackets.

A ductus side-entry jacket mounted to a substrate ductus can allow undiverted flow through the ductus, or by projection of a diversion chute into the luminal stream, divert flow from the ductus, such as a vessel or a ureter, out through a side stem and into a second outlet passageway. Driven by a solenoid, this affords switchability of all flow into either of two outlet lines, that native, of another leading into a shunt or bypass. Replacing the solenoid with a servomotor allows continuously variable adjustment in the relative proportion of flow through either outlet responsive to physiological data fed to the totally implanted system control module executing a patient-specific prescription-program.

The side stem projects perpendicularly, or if angled, then off-perpendicularly from the long axis of the ductus and the jacket encircling it which in compact anatomy, may protrude into neighboring tissue. Usually, this problem can be dispelled with cautious positioning and the placement of padding at the interfaces with the adjoining tissue. Switchable capability has the advantage, for example, of allowing flow to be directed around a segment of a ductus while healing, facilitated by the direct release of medication into the ductus through a jacket accessory channel.

Bypass of a diseased segment can also be followed with excision of the diseased native segment so that the bypass is prosthetic. While incapable of switching, and therefore of supporting a switch type organ transplant, inline coupling jackets are another means for replacing a diseased, malformed, or missing segment of a ductus without interruption in the flow of blood and without sideways protrusion that could prove problematic.

To insert a synthetic segment along a vessel where the volume of flow therethrough need not be regulated does not require diversion which may serve as a bar to positioning in compact anatomy. An inline coupling jacket allows the interpositioning of a length of synthetic tubing along a vessel but in itself lacks means for adjusting the volume of flow-through. An end-inserted device such as that shown in FIG. 31B can be inline without sideways projection but not only lacks means for adjust the volume of flow through but requires that the flow of blood be stopped during placement, thus requiring means of which each introduces risk.

Described in the section to follow, a segment-bridging tie-line prosthesis, elaborated to incorporate a conformation true to the normal anatomy, is used to replace a congenitally malformed aortic arch affected by an aneurysm, coarctation, or interrupted arch in an infant. Because aneurysms may be, and coarctations and interrupted arches are congenital, a fundamental consideration in designing such a prosthesis is not only bloodless placement without the need for cardiopulmonary support but the incorporation of features that allow the prosthesis to expand with growth to an extent that even in a neonate, the need for revision should not arise for an interval more than sufficient for the patient to acquire the robustness that will materially reduce the risk in revision.

In this case, revision consists of replacing the original prosthesis with one larger and able to expand to adult size. Here again, the prosthesis is placed bloodlessly under regional anesthesia by bypassing, or bridging over, the original. The repair of defects of the thoracic and/or thoracoabdominal aorta through the placement of a commercial prosthesis, whether one used to perform a patch aortoplasty, endovascular stent, or a combination of these is more complicated and involves greater risk than does the uninterrupted and sudden transfer of blood flow from the native to the prosthetic lumen of the type thoracic aortic prosthesis described herein.

Apart from surgical and anesthesiologic technique, stopping the flow of blood, much less this close to the brain, is associated with considerable risk. Here the object is to achieve the quick and bloodless interposition of a prosthesis along the thoracic aorta without interruption in the flow of blood or encroachment upon neighboring structures which will remain intact and leak-free indefinitely, and unlike previous commercial or improvised prostheses, accommodate considerable growth allowing placement in a neonate or infant who would otherwise face remediation using a more dangerous method.

To this end, inline ductus coupling jackets allow the interposition of a prosthetic segment along a native vessel. More specifically, inline coupling jackets are nonadjustable upstream native-to-catheteric and downstream catheteric-to-native lumen connectors placed without the need to suspend blood flow and without medically significant bleeding. Also unlike side-entry valve-jackets, inline jackets do not require the removal of a plug of tissue from the side of the substrate ductus, and omit a side stem which even when inclined, presents some sideways protrusion that can disallow placement in compact anatomy. Thus, even though lacking a valving capability and therefore incapable of supporting an intercorporeal procedure such as a metered switch organ transplant, inline coupling jackets offer considerable utility.

Specific information as to details of structure deferred to section 6 below entitled Description of the Preferred Embodiments, shown in FIG. 36A, inline coupling jackets are paired, an entry jacket joining the native vessel to the synthetic conduit of the prosthesis and an exit jacket rejoining the conduit to the native vessel, here the thoracic aorta. For the purpose of assigning the nomenclatural designations that follow, the native vessel transection mechanism shown in FIGS. 36C and 37C are part of the inner half-jackets.

Referring now to FIGS. 36E 37A, and 37B, the inline jackets as a whole—to include both entry inlet half 199 and outlet exit half 200 and corresponding but reversed exit jacket parts 200′ and 199′—are labeled 195 and 195′. The entry and exit surfaces of each half-jacket as specified just below are shown in FIG. 36E. FIG. 36E also shows the part numbers used to denote the eight successive upstream and downstream surfaces of the inline coupling jackets used to secure an aortic neoarch or a tubular tie-line through which blood passes in the direction of arterial flow from left to right as shown. These surfaces are the:
1. Inlet surface of the inlet, or aortic, half of the entry jacket (ISIHEn.J/ISAHEnJ), part number 207.
2. Outlet surface of the inlet, or aortic, half of the entry jacket (OSIHEnJ/OSAHEnJ), part number 208.
3. Inlet surface of the outlet, or conduit, half of the entry jacket (ISOHEnJ/ISCHEnJ), part number 209.
4. Outlet surface of the outlet, or conduit, half of the entry jacket (OSOHEnJ/OSCHEnJ), part number 210.
5. Inlet surface of the inlet, or conduit, half of the exit jacket (ISTHExJ/ISCHExJ), part number 211.
6. Outlet surface of the inlet, or conduit, half of the exit jacket (OSTHExJ)/OSCHExJ), part number 212.
7. Inlet surface of the outlet, or aortic, half of the exit jacket (ISOHEX.J/ISAHEx.J), part number 213, and the
8. Outlet surface of the outlet, or aortic, half of the exit jacket (OSOHEx.J/OSAHExJ), part number 214.

Within the paired jacket pairs, the cutting blade is affixed to the half-jacket that grasps the tie-line, or synthetic conduit, that is, the inlet surface of the conduit entry jacket and the outlet surface of the inlet, or conduit, half of the exit jacket. To simplify the action through which the prosthesis is made to automatically replace the native segment of the substrate vessel without interrupting the flow of blood or bleeding-here a defective aortic arch to be replaced by a prosthetic neoarch—the transection and rotation use the cutting and rotating mechanism affixed to the half-jacket surfaces at the opposite ends of the prosthesis, or tie-line, that is, the outer end-faces of the half-jackets attached to the prosthesis, not those attached to the native vessel, or ductus.

The same action pertains to all of the inline coupling jackets. In an aortic neoarch such as shown in FIG. 36F, this includes one paired prosthesis entry jacket, three paired supra-aortic branch exit jackets, and one paired prosthesis exit jacket. For a replacement aortic arch such as that shown in FIG. 36F, not just situated where the output from the left ventricle represents the maximum pressure in the circulatory system and including branches to replace the proximal segments of the supra-aortic arteries, it is crucial that antegrade migration of the prosthesis responsive to contact with the passing systoles against the inlet surfaces of the inlet halves of the entry and exit jackets not progressively shift these away from the native stumps.

To this end, FIG. 37D shows the through-and-through cross-stitching used to fix all prosthesis, meaning tie-line entry and exit outer half-jackets in place. The number of stitches must not exceed that needed to prevent dislodgement. Migration counteractive measures do not, therefore, depend upon the fact that the foam lining both entry and the exit coupling valve-jackets from end to end complies with, that is, yields to, the passing pulse, and the entry jacket grasps the aorta at or along the entry into the curve while the exit jacket grasps it at or along the exit from the curve, migration is substantially ruled out. Anticlot measures consist of using clot-repellent suture and the automatic release of a drop of heparin. In a patient prone to clotting, the timing of the latter can be made to respond to a signal from a thin film clot detecting sensor positioned along the inner wall of the tie-line, or prosthesis, lumen

Moreover, in this application meant to eschew a need for revision from infancy to adulthood, if based upon laboratory experimentation, it becomes apparent that the pulse is eventually able to shift the inline coupling jackets downstream through the gradual cutting of slots by the cord into the vessel wall, then an additional migration-counteractive measure consists of emulating the tines of an inferior vena cava filter through the use of small thin stainless spring steel wire migration-preventing ‘fishhooks’ to extend from the inlet surface of the inlet half of the entry jacket down to in order to grapple the surface of the substrate adventitia upstream to the jacket. This last measure is never to be the only means for preventing migration, the precedent of inferior vena cava filters cited documented to migrate more than rarely, and to distant landings, which eventuality would be dispelled by using the cross-stitching through the inner half-jackets shown in FIG. 37D.

Furthermore, inline coupling jackets, as do all side-entry jackets and valves, can incorporate suture loops 15 through which suture can be passed to fasten the jacket to neighboring tissue thereby clearing the way for neighboring anatomy. This is likely to cause some discomfort, but only until the connections become encased within a fibrous capsule. More specifically, the tips hooked into the surface of the vessel or neighboring tissue initially provoke a foreign body reaction that ends with their isolation within a fibrous capsule.

2b(2)(c)(c2). Automatic Insertion into a Ductus of an Inline Coupling Jacket-Secured Bridging, or Tie-Line and its Replacement if Necessary

The procedure for replacing a segment along a ductus begins as shown in FIG. 37A with the outer halves of inline coupling jackets 207 and 214 having been snapped shut about what will become the terminal segments of the stump at either end. The mechanism used to effectuate the automated insertion of a vascular prosthesis, which may include branches, is delineated below in section 6, entitled Description of the Preferred Embodiments of the Invention, and is shown in FIGS. 37A thru 37F. The tie-line (bridge, or if anatomically formed, prosthesis) is suspended in the elevated position by the inner inline coupling half jackets at either of its ends. FIG. 37A shows an inline jacket-secured tie-line prosthesis at the start of prosthesis insertion with the outer native ductus-clutching half-jackets having been attached, that is, noncompressibly clamped onto, the native ductus.

At the same time, the inner half-jackets are raised in the elevated position in preparation to descend upon the native ductus, whereupon the serrated razor at the outward-looking end surface of each half-jacket as shown in FIG. 37C will transect the native ductus at either end. The driving hinge-spring at the rotatory joint connecting the axle halves will continue to force the inner jacket halves downward to rotate the tie-line into the position of the native segment. Just as alignment is reached, the ends of the tie-line enter into the ends of the native stumps and this engagement stops along with a detent in the rotatory joint prevents further downward rotation.

Referring to FIG. 37A, for brevity, the elevated half-jackets are the inner half-jackets, or simply, the inner jackets, and the clamping jackets the outer half-jackets, or simply the outer jackets. Entry and exit paired inline coupling jackets are antipodal—that is, identical, but oriented in opposing directions along their common longitudinal axis which passes through the conduit connecting the two; entry and exit jackets are identical where the outlet jacket or jackets are oriented in reverse to the entry jacket. A tie-line is secured at either end by a pair of double half-jacket comprised inline coupling jackets—the vessel-grasping inlet half of the entry jacket and the tie-line grasping outlet half of the entry jacket, then at the outlet end, the inlet and outlet halves of the exit jacket, which is the same but reversed.

There is always a single-entry jacket, and one jacket for each outlet, or branch. In the tie-line, or portion of the prosthesis connected at either end by coupling jackets such as that shown in FIG. 36F, there is one entry and four exit double jackets, one at the inline exit of the prosthesis, and three at the exits of the supraaortic branches. The need to interrupt the flow of blood is eliminated through precise synchronization of action among the jackets. The mechanism always tested before use, the spring-hinges that cause the serrated transection razor to descend upon the substrate ductus are released by vibration motor insertion keys actuated by the same switch.

Turning now to FIG. 37A, rotation of the entry and exit jackets cuts through the substrate ductus as the prosthetic portion or tie-line is rotated into its position. In this way, the flow of blood through the ductus is gradually transferred from the native to the prosthetic lumen. This continued gradual transfer of the blood column in cross-section between the native and prosthetic lumina exerts no significant effect on the blood pressure. Because the sum of the fractional volumes divided between the substrate ductus and the prosthesis are directed alike and constant, that one could interrupt or stop this rotation to allow a controllable and continuously variable fraction of the blood flowing through the substrate vessel to pass through the prosthesis does not justify designation of inline coupling jackets as valves.

The components and action used to insert the prosthetic segment previously occupied by the corresponding segment of a native ductus into the same position were introduced above in section 2, entitled Background of the Invention. To allow each inline coupling jacket half to be opened, each is horizontally transected, the upper and lower halves connected at the back to an axle that spans across and mounts hinges for both, that on the outlet half of the entry jacket is spring-loaded, that on the inlet half of the entry jacket not, this order reversed in the exit jacket.

While a bar connecting the inner half-cylinders of the inline coupling jackets would allow the operator to simultaneously transect both ends of the substrate native vessel manually without the need for an automatic mechanism, without the high-speed reciprocation of the serrated razor, rather than to obtain clean cuts without deformation, the upper edges of the native stumps would be pushed downward and deformed.

A detailed description of this mechanism is delineated under section 6 below entitled Description of the Preferred Embodiments of the Invention, in the subsection entitled Artificial Aorta and Its Automatic Placement and shown in FIGS. 37A, 37C, FIGS. 37A, 37E and 37F show the mechanism that severs the substrate diseased segment while the spring-hinge rotates the prosthetic segment into its place continuously with no interruption in the flow of blood. In this action blood flows gradually transferred from the native diseased to the prosthetic segment such that the sum of blood flow volume between the two is constant.

Referring to FIG. 36A, the prosthetic lumen is lined by a surrounding layer of dissolution-prevention coated viscoelastic polyurethane or similar foam between the outer casing and the stumps of the native ductus encircled within the outer halves of the jackets. As shown in FIGS. 36C, 36E, 37A, and 37C, positioned above the substrate native vessel such as a segment along the thoracic aorta, miniature reciprocating bandsaw-edged razors 226 mounted ahead of the replacement segment S on the inlet surface of the outlet half of the entry jacket and the outlet surface of the inlet half of the exit jacket transect the substrate native vessel at either end.

Unlike a compound vascular bypass, or sudden switched-organ transplantation, transplantation of a solid organ where the valves regulating the reciprocal cross-circulation between the donor and recipient are controlled by a microcontroller- or in comorbid disease, requires a high level of coordinated treatment recommending the assignment of each comorbidity to a dedicated arm in a hierarchical control system, a microprocessor—the insertion of an inline coupling jacket-connected tie-line or a prosthesis to replace a defective or diseased segment along a ductus proceeds to a significant extent automatically by spring-driven clockwork-like mechanical means which eliminate the need for electronically based automatic control.

The outlet half encircles the upstream, or retrograde, end of the tie-line or prosthetic replacement segment which will be rotated into alignment with the native ductus. The outlet half accomplishes this with a serrated razor reciprocated to cut through the native ductus while rotating the synthetic segment into alignment with the cut end of the native ductus held in the inlet jacket half in one continuous operation. The razor is urged downward by the spring hinge on the outlet half which causes that half to close and lock the outlet half in the same type lock automatically as was locked manually on the inlet half.

Branches such as the supraaortic are replaced with a unitary prosthesis that supplicates the anatomy of the aortic arch with its branches. The termini of the branches are attached in the same action that replaces the arch, each branch with its own double half cylinder inline coupling jacket. Precise synchronization is obtained by switching the five double half cylinder inline coupling jacket from the same switch.

The reciprocating action used to transect the native ductus at both entry and exit ends is produced by small cellular telephone-type vibratory motors to the back of the pin jacks, powered by button cell batteries in the operator hand control. The pin jacks fit in sockets with openings at the surface of each jacket so that when fully inserted, come into contact with the rear of the razor blades. Synchronization between the entry and exit jackets—in an aortic neoarch, the entry and exit jackets defining the segment along the arch and the normally three supra-aortic branches—is achieved by controlling all the vibratory motors as one with the one switch on the hand control.

As the alternating flats 233 and vaults 234 structure to allow expansion for growth denote, FIG. 36F shows a replacement aortic arch for use in an infant or toddler. Replacement of a segment along a great vessel such as the thoracic aorta can supplant more than one defect, whether an aneurysm, pseudoaneurysm, incision, coarctation, or interruption, and can replace an anomaly in the same procedure. No less important, replacement of the affected segment precludes a recurrence. A prosthesis to replace an aneurysm-prone aortic arch, for example, can at the same time correct an anomalous left common carotid that originates from the pulmonary artery, for example, in one operation.

Provided the distal course of the carotid is normal and only the origin—usually from the pulmonary artery—is anomalous, the carotid is jacketed distal to the anomaly in the common carotid reentry half of the exit jacket and the defective origin closed off (see, for example, Sachdeva, S., Gupta, S. K., Rajashekar, P., Jagia, P., and Devarajan Sebastian, L. J. 2022. “Isolated Left Common Carotid Artery: Multidisciplinary Management is the Key to Success,” Cardiology in the Young (Cambridge, England) 32(9): 1-8; Cohen, J. L., Sranford, N., and Torres, A. 2019. “Isolated Left Common Carotid Artery in an Infant with Pulmonary Atresia and Intact Ventricular Septum,” Online, Annals of Pediatric Cardiology 12(2): 156-158; Hanneman, K., Newman, B., and Chan, F. 2017. “Congenital Variants and Anomalies of the Aortic Arch,” Radiographies 37(1): 32-51; Osakwe, O., Jones, B., and Hirsch, R. 2016. “Anomalous Origin of the Left Common Carotid Artery from the Main Pulmonary Artery: A Rare Association in an Infant with CHARGE [Coloboma, Heart defects, Atresia choanae, Retarded growth, malformed Genitalia, and statoacoustic and ol factory impairment of the Ear nose respectively] Syndrome,” Online, Case Reports in Pediatrics 2016:2064937; Setacci, F., Singnano, P., de Donato, G., Chisci, E., Galzerano, G., and Setacci, C. 2009. “Anomalies and Variant Anatomy of the Aorta and the Supra-aortic Vessels: Additional Challenges Met by Hybrid Procedures,” Online, HSR [Health Services Research] Proceedings in Intensive Care and Cardiovascular Anesthesia 1(1): 37-44; Kau, T., Sinzig, M., Gasser, J., Lesnik, G., Rabitsch, E., and 4 others 2007. “Aortic Development and Anomalies,” Online, Seminars in Interventional Radiology 24(2): 141-152).

Not presenting one or more defects along a singular vessel, a double aortic arch, whereby the aorta develops as two branches which encircle the trachea and/or esophagus to pose a risk of suffocating the neonate or baby, is not amenable to Automatic repair through replacement of the segment by an inline coupling jacket secured prosthesis. Variable and intricate, such a ‘vascular ring’ demands repair by an experienced operator (see, for example, Tarmahomed, A. and Umapathi, K. K. 2022. “Double Aortic Arch,” Online, Treasure Island, Florida: StatPearls Publishing Company).

However, while limited to the replacement of a unitary substrate ductus, as the replacement aortic arch shown in FIG. 36F indicates, other deviations from cylindrical such as branches can be accommodated. To apply an inline coupling jacket secured prosthesis in the replacement of malformed or diseased vasculature, the prosthesis such as that for an aortic arch shown in FIG. 36F is positioned above as superimposed with respect to the section to be replaced.

The inlet half of the entry jacket is connected at the entry into the vascular section to be replaced, and the outlet half of the exit jacket for each of the branches and the section as a whole are connected to the section outlets. The automated process whereby the native section is transected and the prosthesis rotated into its position and secured is described above in section 2b(2)(c)(c2), entitled Automatic Insertion into a Ductus of an Inline Coupling Jacket-secured Bridging, or Tie-line, and Its Replacement if Necessary, and shown in FIGS. 37A thru 37F.

Each reciprocating bandsaw razor is connected to a small compression spring to its rear separate from but next to the spring hinge so that the vibration applied to the razor which moves it forward is countered by the compression spring causing the razor to reciprocate at high speed in the manner of a reciprocating saw or jigsaw. Under the half-jacket closing force of the spring hinges, the outlet half of the entry jacket and the inlet half of the exit jacket, grasping the opposite ends of the synthetic replacement segment, will rotate the reciprocating serrated razors to transect the native ductus at either end and situate its synthetic replacement in line with the stumps of the native ductus.

It might seem necessary to compensate for the time blood would take to transit the segment between the jacket halves clutching the native ductus at either end as well for the minute interruption in the transfer of blood flow from the native to the synthetic ductus caused by the distance separating the razor cutting and noncutting edges for the inlet half of the exit jacket to be energized at a slight interval after the outlet half of the entry jacket. However, the flow of blood never interrupted and continuously transferred from the native to the prosthetic ductus, these intervals and those for the blood to pass into and through the supra-aortic branches of the prosthesis, for example, are far too small to induce a stroke or other adverse complication. Accordingly, the same action proceeds simultaneously at both the entry and exit jackets.

The jacket is positioned along the native ductus by opening both inlet and outlet halves, the inlet half manually closed to encircle the ductus and manually locked in place with a small briefcase-type lock. Referring to FIG. 37A, the inlet half of the entry jacket and outlet half of the exit jacket which clutch the native ductus at either end are not rotated but stationary, it is the outlet half of the entry jacket and the inlet half of the exit jacket that will rotate the razor ahead of the replacement ductus to position it where the native ductus was before having been transected. At the outset of segment replacing procedure, the ductus encircled in the outlet half of the entry jacket and the inlet half of the exit jacket is the native segment while that at the completion of the procedure is the prosthetic segment.

Fully inserting the pin jacks while still stationary into the pin jack sockets so that these come into contact with the back of the spring retum-mounted serrated razors and only once this has been done is switching on the vibratory motors retracts the trips that had prevented premature contact between the razor and initiates the cutting action. A deviation from simultaneity in the initiation of the cutting action at either end of the segment to be replaced would likely result in problem bleeding. With a vessel that supplies or drains the brain or the heart, this could prove dire. Accordingly, this sequence in the steps taken assures that the cutting action will be exactly synchronous at both ends of the segment to be replaced.

As delineated above in the section entitled Background to the Invention, two mechanisms protect the native ductus from premature incision by the serrated razor. The first are trips in each jacket which are extended when the outlet half of the entry jacket and inlet half of the exit jacket are opened. These limit closure of the jackets, hence the razors, to a safe distance above the native ductus. These trips are not released until the pin jacks that will drive the razors have been fully inserted into the pin jack sockets and current switched on to initiate the cutting action. The other protective mechanism is that the spring hinges used in the outlet half of the entry jacket and inlet half of the exit jacket are sufficiently damped to bring the blade against the aorta gently so that the other measures taken to prevent a premature incision must be understood as provided out of an abundance of caution.

In use, the upstream entry and downstream exit jackets are manually locked in position at the levels that define the entry and exit of the segment to be replaced. The jacket halves are retained in position by small locks of the briefcase type when the intromitting pawl from one half of the lock is entered into its receiving half and fixed therein by a spring-loaded pawl undercutting arm. Imperative to thwart a stroke, in an aortic neoarch, continuity of blood flow through the aorta and the supra-aortic branches is sustained throughout, because the flow of blood is transferred from the native arch to its prosthesis. For this reason, while highly improbable, were the process for any reason interrupted, flow would continue according to the apportionment between the two passageways established at the time of the interruption.

Positioned with its backside against the front, or inlet surface of the outlet, or conduit, half of the neoaortic entry jacket, for example, the serrated razor precedes the proximal end of the one-millimeter extended conduit. The outlet half with serrated razor is set in motion when the control switch is depressed to release the trip and initiate the reciprocating cutting action of the native ductus by the serrated razor. Inlet and outlet halves of each inline coupling jacket are united by a common hinge along one side. The stationary jacket halves manually closed and locked shut before the cutting action is begun, highly damped spring hinges are provided only for the rotating native to synthetic ductus exchanging outlet half of the entry jacket and the inlet half of the exit jacket.

When the cutting action is initiated, the damped spring-hinged outlet half of the entry jacket and its exit counterpart are simultaneously freed by withdrawal of the trips to continue their nonabrupt descent upon to cut through the native ductus. Upon alignment of the spring-loaded pawl in the receiving half of each jacket outlet half lock with the undercut in the male intromitting member the outlet halves have been automatically locked shut under the spring directed action of the jacket half spring hinges and the springs in the locks.

Even in an infant, the expandability of the prosthesis to accommodate growth through mechanical and inherent elasticity should be sufficient to avoid the need for later revision. Nevertheless, if revision does become necessary, the existing prosthesis is bypassed by positioning the inline coupling entry jacket of the larger replacement prosthesis upstream and the exit jacket downstream to those of the existing prosthesis, the process little different than that used to place the original prosthesis.

The same positioning of the entry jacket upstream, and the exit jacket downstream along a unitary aorta which tortuous, compresses the esophagus (Orringer, M. B. 1997. “Tumors, Injuries, and Miscellaneous Conditions of the Esophagus,” in Greenfield, L. J., Mulholland, M., Oldham, K. T., Zelenock, G. B., and Lillemoe, K. D., (eds.) Surgery: Scientific Principles and Practice, Philadelphia, Pennsylvania: Lippincott Raven, chapter 19, page 730), or before and after a double aortic arch divides to enclose the trachea and/or esophagus, trapping and often constricting these within a ‘vascular ring’ is used to eliminate a double aortic arch.

The prosthesis includes the supra-aortic branches, so that a doubling of the common carotid and subclavian arteries, a missing brachiocephalic, or a complete ring to compress the esophagus formed “by the ascending aorta and pulmonary artery anteriorly, with the aortic arch on the right and either the ductus arteriosus or the ligamentum arteriosum and the left subclavian artery on the left,” (Coran, A. G. and Oldham, K T. 1997, “in Greenfield, L. J., Mulholland, M., Oldham, K. T., Zelenock, G. B., and Lillemoe, K. D., (eds.) Surgery: Scientific Principles and Practice, Philadelphia, Pennsylvania: Lippincott Raven, Chapter 102, pages 2016-2017) are corrected at the same time.

Defects of the thoracic aorta highly variable, depending upon the specific circumstances, the use of a prosthesis to allow repair of almost any variant, some adjunctive surgery may be necessary (see also, for example, Backer, C. L., Bharadwaj, S. N., Eltayeb, O. M., Forbess. J. M., Popescu, A. R., and Monge, M. C. 2019. “Double Aortic Arch with Kommerell Diverticulum,” Annals of Thoracic Surgery 108(1): 161-166; Campanale, C. M., Pasquini, L., Santangelo, T. P., Iorio, F. S., Bagolan, P., Sanders, S. P., and Toscano, A. 2019. “Prenatal Echocardiographic Assessment of Right Aortic Arch,” Ultrasound in Obstetrics and Gynecology 54(1): 96-102; Young, A. A., Hornberger, L. K., Haberer, K., Fruitman. D., Mills, L., and 3 others 2019. “Prenatal Detection, Comorbidities, and Management of Vascular Rings,” American Journal of Cardiology 123(10): 1703-1708; Priya, S., Thomas, R., Nagpal. P., Sharma, A., and Steigner, M. 2018. “Congenital Anomalies of the Aortic Arch,” Cardiovascular Diagnosis and Therapy 8 (Supplement 1): S26-S44; Wang, Y., Fan, M., Siddiqui, F. A., Wang. M., Sun, W., and 3 others 2018. “Strategies for Accurate Diagnosis of Fetal Aortic Arch Anomalies: Benefits of Three-dimensional Sonography with Spatiotemporal Image Correlation and a Novel Algorithm for Volume Analysis,” Journal of the American Society of Echocardiography 31(11): 1238-1251; François, K., Panzer, J., De Groote, K., Vandekerckhove, K., De Wolf, D., and 5 others 2017. “Early and Late Outcomes after Surgical Management of Congenital Vascular Rings,” European Journal of Pediatrics 176(3): 371-377; Naimo, P. S. Fricke, T. A., Donald, J. S., Sawan, E., d'Udekem, Y., Brizard, C. P., and Konstantinov. I. E. 2017. “Long-term Outcomes of Complete Vascular Ring Division in Children: A 36-year Experience from a Single Institution,” Interactive Cardiovascular and Thoracic Surgery 24(2): 234-239; Riggle, K. M., Rice-Townsend, S E., and Waldhausen, J. H. T. 2017. “Thoracoscopic Division of Vascular Rings,” Journal of Pediatric Surgery 52(7): 1113-1116; Backer, C. L., Monge, M. C., Popescu, A. R., Eltayeb, O. M., Rastatter, J. C., and Rigsby, C. K. 2016. “Vascular Rings,” Seminars in Pediatric Surgery 25(3): 165-175; Garcia-Guereta, L., Garcia-Cerro, E., and Bret-Zurita, M. 2016. “Multidetector Computed Tomography for Congenital Anomalies of the Aortic Arch: Vascular Rings,” Revista Española de Cardiologia (English edition) 69(7): 681- 693; Trobo, M. D., Bravo, C., Lancharro, A., Gamez Alderete, F., Marin, C., and de Leon-Luis, J. 2016. “Neonatal Magnetic Resonance Imaging in Double Aortic Arch Diagnosed Prenatally by Ultrasound,” Journal of Obstetrics and Gynaecology 36(4): 526-528; Lee, J. H., Yang, J. H., and Jun, T. G. 2015. “Video-assisted Thoracoscopic Division of Vascular Rings,” Korean Journal of Thoracic and Cardiovascular Surgery 48(1): 78-81; Nezafati. M. H. and Nezafati. P. 2015. “Video Assisted Thoracoscopic Surgery Cases with Right-sided Aortic Arch Aneurysm and Complete Vascular Ring: Case Report,” International Journal of Surgery Case Reports 6C: 188-190; Trobo, D., Bravo, C., Alvarez, T., Perez, R., Gamez, F., and De Leon-Luis, J. 2015. “Prenatal Sonographic Features of a Double Aortic Arch: Literature Review and Perinatal Management,” Journal of Ultrasound in Medicine 34(11): 1921-1927; Backer, C. L., Monge. M. C., Russell, H. M., Popescu, A. R., Rastatter, J. C., and Costello, J. M. 2014. “Reoperation after Vascular Ring Repair,” Seminars in Thoracic and Cardiovascular Surgery Pediatric Cardiac Surgery. Pediatric Cardiac Surgery Annual 17(1): 48-55; Bai, S., Li, X. F., Liu, C. X., Peng. Y., Yuan, F., and 4 others 2012. “Surgical Treatment for Vascular Anomalies and Tracheoesophageal Compression,” Chinese Medical Journal (English edition) 125(8): 1504-1507; Tehrai, M., Saidi, B., and Goudarzi, M. 2012. “Multi-detector Computed Tomography Demonstration of Double-lumen Aortic Arch—Persistent Fifth Arch—as an Isolated Anomaly in an Adult,” Cardiology in the Young (Cambridge, England) 22(3): 353-355; Axt-Fliedner, R., Kawecki, A., Enzensberger, C., Wienhard, J., Degenhardt, J., Schranz, D., and Vogel, M. 2011. “Fetal and Neonatal Diagnosis of Interrupted Aortic Arch: Associations and Outcomes,” Fetal Diagnosis and Therapy 30(4): 299-305; Dillman, J. R., Attili, A. K., Agarwal, P. P., Dorfman, A. L., Hernandez, R. J., and Strouse, P. J. 2011. “Common and Uncommon Vascular Rings and Slings: A Multi-modality Review,” Pediatric Radiology 41(11): 1440-1454, 1489-1490; Yildirim, A., Karabulut, N., Doğan, S., and Herek, D. 2011. “Congenital Thoracic Arterial Anomalies in Adults: ACT Overview,” Diagnostic and Interventional Radiology (Ankara, Turkey) 17(4): 352-362; Tuo, G., Volpe, P., Bava, G. L., Bondanza, S., De Robertis, V., Pongiglione, G., and Marasini, M. 2009. “Prenatal Diagnosis and Outcome of Isolated Vascular Rings,” American Journal of Cardiology 103(3): 416-419; Humphrey, C., Duncan, K., and Fletcher, S. 2006. “Decade of Experience with Vascular Rings at a Single Institution,” Pediatrics 117(5): e903-e908; Shah, R., Mora, B., Bacha, E., Sena, L., Buonomo, C., Del Nido, P., Rahbar, R. 2006. “The Presentation and Management of Vascular Rings: An Otolaryngology Perspective,” International Journal of Pediatric Otorhinolaryngology 71(1): 57-62; Soares, G., Alvares, S., Rocha, C., Teixeira, M. F., Mota, M. C. R, and 4 others 2005. “Congenital Heart Defects and Chromosomal Anomalies Including 22q11 Microdeletion (CATCH 22),” Revista Portuguesa Cardiologia [Portuguese Journal of Cardiology] (Lisbon, Portugal) 24(3): 349-371; Bonnard, A., Auber, F., Fourcade, L., Marchac, V., Emond, S., and Revillon, Y. 2003. “Vascular Ring Abnormalities: A Retrospective Study of 62 Cases,” Journal of Pediatric Surgery 38(4): 539-543; Greiner, A., Perkmann, R., Rieger, M., Neuhauser, B., and Fraedrich, G. 2003. “Vascular Ring Causing Tracheal Compression in an Adult Patient,” Annals of Thoracic Surgery 75(6): 1959-1960; McElhinney, D. B., Clark, B. J. 3rd, Weinberg, P. M., Kenton, M. L., McDonald-McGinn, D., and 3 others 2001. “Association of Chromosome 22q11 Deletion with Isolated Anomalies of Aortic Arch Laterality and Branching,” Journal of the American College of Cardiology 37(8): 2114-2119; Momma, K., Matsuoka, R., and Takao, A. 1999. “Aortic Arch Anomalies Associated with Chromosome 22q11 Deletion (CATCH 22),” Pediatric Cardiology 20(2): 97-102; Beekman, R. P., Hazekamp, M. G., Sobotka, M. A., Meijboom, E. J., de Roos, A., and 3 others 1998. “A New Diagnostic Approach to Vascular Rings and Pulmonary Slings: The Role of MRI,” Magnetic Resonance Imaging 16(2): 137-145; Singh, G. K., Greenberg, S. B., and Balsara, R. K. 1997. “Diagnostic Dilemma: Left Aortic Arch with Right Descending Aorta—A Rare Vascular Ring,” Pediatric Cardiology 18(1): 45-48; Burke, R. P., Rosenfeld, H. M., Wernovsky, G., and Jonas, R. A. 1995. “Video-assisted Thoracoscopic Vascular Ring Division in Infants and Children,” Journal of the American College of Cardiology 25(4): 943-947; Hartenberg, M. A.; Salzberg, A. M.; Krummel, T. M.; and Bush, J. J. 1989. “Double Aortic Arch Associated with Esophageal Atresia and Tracheoesophageal Fistula,” Journal of Pediatric Surgery 24(5): 488-490; Gross, R. E. 1945. “Surgical Relief for Tracheal Obstruction from a Vascular Ring,” New England Journal of Medicine 233:586). In this procedure, once the exit jacket has transected the aorta a moment after the entry jacket had done so, the divided segment now cut away is discarded leaving the singular segment replacing prosthesis having joined the upstream to the downstream aorta.

Its proximal, or entry jacket-bound end rounded and slippery to ride over the surface of the foam without catching, the synthetic central conduit of the native aortic arch, situated just behind the razor, extends retrograde, or upstream, by a millimeter past the inlet surface of the outlet half of the entry jacket, hence, through the outlet surface of the inlet half of the entry jacket, to cut the aorta flush to the plane of the inlet half of the outlet side positioned to the right as shown. Thus, when the five or six cutting halves of the inline coupling jackets close upon to cut through the aortic arch and supra-aortic branches, the aorta and its synthetic continuation have been placed in flush end to end relation.

A more detailed description of the action deferred to the section below entitled Description of the Preferred Embodiments, when the control switch is depressed, the defective segment of the aorta—from the interface separating the inlet from the outlet halves of the upstream, or retrograde entry jacket to the interface separating the inlet from the outlet half of the downstream exit jacket—is excised for retrieval with tweezers or a pliers. Proximal to the carotids, the serrated razors, or razor saws, made of stainless steel or titanium, must cut through the aorta cleanly without generating debris, and the blade must be thin as not to create a significant kerf. If the prosthesis allows for separately jacketing each branch of the brachiocephalic, then the action must proceed in synchrony to include these locations as well.

2b(2)(c)(c3). Types of Inline Coupling Jacket-Secured Bridging, or Tie-Lines

To the extent that branches are included, thoracic, thoracoabdominal, abdominal, and entire aorta prostheses, for example, are anatomically approximate configured tie-lines, or bridges, for replacing a segment which is congenitally or otherwise irreparably defective due to disease or injury. However, most ductus substantially tubular, tie-line prostheses follow suit without tapering or flaring. Accordingly, use of the term “anatomical;” means only that the prosthesis is shaped to replace a branched segment along a vessel such as the thoracic arch with supraaortic branches shown in FIG. 36F or the abdominal aorta at the celiac axis or the renal arteries.

However, as depicted here, it is still tubular in any cross-section without any flaring or tapering as seen in the native structure (Abdullateef, S., Mariscal-Harana, J., and Khir, A. W. 2021. “Impact of Tapering of Arterial Vessels on Blood Pressure, Pulse Wave Velocity, and Wave Intensity Analysis Using One-dimensional Computational Model,” Online, International Journal for Numerical Methods in Biomedical Engineering (Oxford, England) 37(11): e3312; Walburn, F. J. and Stein, P. D. 1981. “Effect of Vessel Tapering on the Transition to Turbulent Flow: Implications in the Cardiovascular System,” Journal of Biomechanical Engineering 103(2): 116-120).

‘Anatomical’ prostheses can include branches smaller in caliber than their trunk, or vessel of origin, to replace a specific segment along a major vessel requiring that these be produced separately from a continuous reel or coil of tubing or a coil or the expandable structure form from which the length needed is cut off. That is, the distinction between unbranched and branched pertains not just to simple nonexpandable tubular tie-line type prostheses for placement in adults but also the expandably structured “tubing” described below in section 2b(2)(f), entitled Expandable Structure to Accommodate Growth and Self-adjust in Size in Vascular Segment Prostheses, Lines, and Confluence Conduits Placed in Children, which can be used in either pediatric or adult patients.

Defects of the thoracic and abdominal aorta most prevalent, ready-made vascular segment replacement prostheses include embodiments that include the supraaortic branches and branched section along the abdominal aorta to include the celiac axis and renal arteries, for example. Other tie-line type prostheses whether tubular or expandable can be provided as coiled for the operator to cut off a long a segment as is warranted by the medical condition.

Whether tubular or growth-accommodable, the inline coupling jackets used to fix the prosthesis in position are the same as shown in FIG. 37A. Focus on the aorta is because other than the heart, it is the structure most often affected by vascular congenital defects. A prosthesis to replace a large vessel is anatomically elaborated if considered hemodynamically significant; otherwise, a simple tube-shaped prosthesis should be used. Branches incorporated into a prosthesis are cylindrical. One congenital cause of an aneurysm is a connective tissue disorder, wherewith an acquired increase in blood pressure will cause the emergent aneurysm to enlarge, warranting the making of an abdominal aortic prosthesis expandable or not which is extended cranially and caudally to include any additional branches traversed.

The qualification specified above understood, an aortic arch prosthesis such as that shown in FIG. 36F is ‘anatomically’ elaborated to the extent of incorporating the supra-aortic branches which supply blood to the head and arms without the slight flaring or tapering of the up- and downstream ends of the prosthesis to facilitate engagement into the stumps when automatically placed in alignment as described below in section 2b(2)(c)(c2), entitled Automatic Insertion into a Ductus of an Inline Coupling Jacket-secured Bridging, or Tie-line and Its Replacement if Necessary.

The methods and apparatus described herein were devised to eliminate the need to interrupt the flow of blood through the affected segment, organ, or volume of tissue, and in so doing, eliminate the numerous and serious risks associated with cardiopulmonary bypass, cardioplegia, deep or moderate circulatory arrest, and general anesthesia, and when pertinent, the additional procedural time required to induce hypothermia and rewarm the patient. This applies no less to vascular prostheses.

While made in one size for manual adaptation to fit the patient, aneurysms, dissections, and/or coarctations or interrupted aortic arches not due to a genetic defect, hence, not prone to spread, justify embodiments shaped to fit in a smaller area; hence, not all aortic prostheses are made to replace the entire aorta. Ischemia the major cause for midprocedural complications and postprocedural adverse sequelae, the primary advantage provided by all side-entry and inline coupling devices is that all are placed without the need to interrupt the flow of blood. This eliminates the complications that can result from the need for cardiopulmonary life support, cardioplegia, and general anesthesia.

Coupling jacket-connected bridging, or tie-lines consist of a vascular prosthesis interposed along a native vessel where said prosthesis can be a simple tube or a major portion of the vascular shaped anatomically. Provided the primary purpose of the tie-line is to replace a diseased or damaged segment along a native ductus, a secondary use for such a tie-line can also serve to mount another device such as electrostimulatory or neuromodulatory. Mounting thus avoids any adverse reaction that would likely result were contact with the substrate direct.

Where a segment along a nonbranched length of a native vessel is replaced, the native and replacement structures are simple tubes, the latter secured in position by an inline coupling jacket at either end. In contrast, replacement of the thoracic aorta, for example, includes the supra-aortic branches. Such an anatomy-impressed prosthesis is secured in place by paired inline coupling jackets at each of its ends. Inline coupling jackets are described above in section 2b(2)(c)(c1), entitled Inline coupling jackets Proper, with the nomenclature for designating the parts of the prosthesis overall shown in FIG. 36E. With respect to the aorta and its branches, the entry into the aortic arch is the only sending paired inline coupling jacket, the others being receiving paired inline coupling jackets.

The distinction between these is simply one of inlet-outlet orientation as up- or downstream. Unlike ductus side-entry jackets, which capable of flow diversion and apportionment between either of two outlets may be independent, or as in an organ transplant, paired, inline coupling jackets are paired as the end-connectors of a replacement segment for a substrate ductus. More specifically, entry and exit jackets fused to and inseparable from the line intervening between the two serve as the terminal connectors to the up- and downstream ends, or stumps, of the substrate ductus, usually a vessel.

### Simple Lesioned Segment-Bridging, or Tie-Lines

A simple lesioned segment bridging tie-line is a prosthetic replacement for a segment along an artery which does not give off branches and not compensated for by collateral circulation. As are all prosthetic replacements for blood-conducting structures in a pediatric patient, to include tie-lines and confluence conduits, the material and valves comprising these must afford a degree of expandability, flexibility, and tensile strength to allow considerable expansion due to growth while posing no risk of dehiscence or rupture.

The structure used is described below in section 2b(2)(f), entitled Expandable Structure to Accommodate Growth and Self-adjust in Size in Vascular Segment Prostheses, Lines, and Confluence Conduits Placed in Children, and shown in FIGS. 36A and 36F expandable inline coupling jackets described above in sections 2b(2)(c), entitled Vascular Segment Prostheses: Inline Coupling Jackets and Bridging, or Tie-lines, thru 2b(2)(e), entitled Tie-line Mounted Side-entry Flow diversion Jackets, Vascular Valves, and Servovalves. In referring to tie-line type vascular prostheses, the terms ‘shape-impressed’ and ‘anatomical’ do not mean that these are literal facsimiles to include any tapering or bends, for example.

For replacing specific segments along major vessels, these are cylindrical with cylindrical branches incorporated and therefore cannot be dispensed on a continuous roll as would branchless tubing or the expandable conduit described shortly below in section 2b(2)(f), entitled Expandable Structure to Accommodate Growth and Self-adjust in Size in Vascular Segment Prostheses, Lines, and Confluence Conduits Placed in Children, for the operator to cut off whatever length is needed. However, because the expandable structure can be manually lengthened and increased in diameter, only one size need be produced.

Standardized thus, an inline thoracic aorta aneurysm and/or coarctation, or interruption replacement prosthesis for use in a prenate or neonate with a severe defect of the aortic arch, for example, would use the same sized prosthesis. Depending upon the size range involved, this same sized prosthesis is likely to fit smaller adults. A larger version of the same prosthesis would differ only in size. An intermediate size is probably unnecessary but readily accommodable. Accordingly, ‘anatomical’ in this context means that the bridge or tie-line is true to the form of the native vessel to be replaced only to the extent that it is cylindrical rather than tapered or flared or bent, and to replace a particular segment includes its branches which are smaller in diameter than the trunk or vessel of origin.

In the chest, this comprises the supraaortic arteries of the aortic arch leading into the head and arms, while segments along the abdominal aorta which may require replacement include that at or near the celiac axis or the renal vessels, for example. There is no celiac vein. The supraaortic veins are more consolidated and fewer than the arteries but overall conformant in distribution with them. Tie-line type vascular prostheses are no less applicable to the replacement of malformed veins than arteries (see, for example, Bala, V. P., Barathi, S. D., and Govindarajalou, R. M. S. 2023. “Multidetector Computed Tomography (MDCT) Angiography Evaluation of Total[ly] Anomalous Pulmonary Venous Connection,” Online, Cureus (Palo Alto, California) 15(10): e46852; Idries, I. Y., Sur, A., Yadav, R., Yadav, V., Jaswani, V., and Zaman, M. A 2023. “Rare Presentation of Duplicate Superior Vena Cava Precipitating Premature Atrial Contractions: A Case Report and a Review of the Literature,” Cureus (Palo Alto, California) 15(4): e37791; Masino, F., Muscatella, G., Montatore, M., Gifuni, R., and Guglielmi, G. 2023. “A Remarkable Case Report of an Interrupted Inferior Vena Cava with Hemiazygos and Transhepatic Continuation,” Online, Acta Biomedica (Fidenza, Parma, Italy) 94(5): e2023238; Bae, S. B., Kang, E. J., Choo, K. S., Lee, J., Kim, S. H., Lim, K. J., and Kwon, H. 2022. “Aortic Arch Variants and Anomalies: Embryology, Imaging Findings, and Clinical Considerations,” Online, Journal of Cardiovascular Imaging (Seoul, South Korea) 30(4): 231-262). Orozco, V. M., Ricardo, U., Carlos, H., Paula, M., and Katherine F. 2022. Thoracic Vascular Variants and Anomalies: Imaging Findings, Review of the Embryology, and Clinical Features,” Online, The Indian Journal of Radiology and Imaging (Stuttgart, Germany) 32(4): 568-575; Kwak, J. G., Kim, S. J., and Lee, C. H. 2011. “A Rare Case of Partial Anomalous Pulmonary Venous Return to Azygos Vein Without Direct Connection to Great Cardiac Vessels,” Online, Annals of Thoracic Surgery (Amsterdam, North Holland) 92(3): e51-e52; Demos, T. C, Posniak, H. V., Pierce, K. L., Olson, M. C., and Muscato, M. 2004. “Venous Anomalies of the Thorax,” Online, American Journal of Roentgenology (Leesburg, Virginia) 182(5): 1139-1150).

Exceptionally, both the heart and great vessels may present congenital defects, making it best to transplant the graft organ together with its great vessels. Otherwise, congenital defects of the thoracic and thoracoabdominal aorta alone are more common and may be repaired with a prosthesis of the kind described and illustrated in the section 6 below, entitled Description of the Preferred Embodiments of the Invention.

### Anatomical Prostheses

Use of the term ‘anatomical’ to describe a tie-line type vascular prosthesis as approximately so having been qualified above in this section, FIG. 36F shows an aortic neoarch as an example of an anatomically elaborated, or shaped, prosthetic bridging tie-line, where the incorporation of supra-aortic arterial branches calls for the addition, albeit tiny, of another three to five passive inline coupling jackets. For use in the very young, any type of inline coupling jacket-secured bridging, or tie-line can be made to expand with growth. This factor makes possible an initial correction that spontaneously adapts to the growing size of the patient, is durable; and supported by one or more accessory channels that allow the direct targeting to the device and its connecting jackets of anticoagulative, antimicrobial, vasodilatory or any other kind of medication.

Moreover, made of synthetic materials, a prosthesis is unsusceptible to infection, requires no blood supply or drainage, and suffers nothing due to loss of innervation and the sensation of pain that normal tissue generates. Provided with secretants such as vasodilatory by the endothelium lining the entire vascular tree and the urothelium lining the urinary tract, the blood normally includes these in sufficient concentration to render the absence of such secretion across the brief extent of the prosthesis of nugatory if any significance. The prosthesis itself has no need for a vasodilator or any other ductus lining-secreted agent.

However, if the patient is deficient in any, the prosthesis can be used as a sensor and drug-release platform itself invulnerable to any adverse effect from a volume of the agent at least slightly greater in concentration than should be put into immediate contact with the target tissue. In this way, the implanted control microprocessor or microcontroller can be programmed to release the necessary agent in sufficient volume to supply the entire vascular or urinary system at a quicker rate than might a drip. Some applications could be served by side-entry devices but at the expense of longer preparatory and procedural time, and likely, within limited space.

While inline coupling jackets may be viewed as serving a valving function in the gradual transfer of flow from the lumen of a native ductus, usually a blood vessel, to the lumen of the tie-line or base-tube that is rotated around to take the place of the native segment after it had been transected at either end, this action is limited to it placement which unlike a valve is a one-time event. The mechanism used to accomplish this transection and replacement is described in the preceding section 2b(2)(c)(c2), entitled Automatic Insertion into a Ductus of an Inline Coupling Jacket-secured Bridging, or Tie-line and Its Replacement if Necessary. Incapable of providing a switching function between either of two outlet channels, inline coupling jackets might be thought of as less versatile than side-entry devices, but such function is unintended and nonessential.

These connectors are relatively simple, and except in size, invariable; variability pertains rather to the tie-lines fixed in position by inline jackets. Inline coupling jackets are relatively simple in mechanism, quicker and easier to place without much experience, widely adaptable in diameter of the ductus to be affected, and expandable as necessary to cover the entire range of sizes from infant to adult. Were ductus side-entry valves and servovalves, as are inline coupling jackets, all alike except for size, to make these available as pre-fused jacket-outlet line combinations would require a large inventory. For this reason, vascular valves and servovalves are, at least ordinarily, separate components.

2b(2)(d). Anoxia-Free Aortic Repair: Aortic Prostheses Thoracic, Thoracoabdominal, and Abdominal

Severe dissections, aneurysms, and congenital coarctations and interruptions all pose potentially fatal progression unless repaired- or better still, replaced-if not immediately, then upon confirmation that the lesion is expanding. The automatic replacement of a defective segment along a vessel, described in terms of the aorta, under the watchful eye of the operator and an assistant will be found above in section 2b(2)(c)(c2), entitled Automatic Insertion into a Ductus of an Inline Coupling Jacket-secured Bridging, or Tie-line, and Its Replacement if Necessary, and shown in FIGS. 37A thru 37F.

The object to replace congenitally malformed or defective vascular tissue in an infant once and for all, and thus do away with conventional prostheses made of textiles having limited expandability that while usable in an adult, will certainly require to be replaced through a procedure likely to require sectioning the sternum and thus causing severe pain. To accomplish a once and for all repair demands seamless connections not susceptible to dehiscence, and more extension that eliminates adjoining tissue that if allowed to remain would likely fail.

Existing methods for repairing a defective major vessel necessitate the interruption of perfusion while on heart-lung life support, which is indissociable from ischemia and the risk of reperfusion injury. Using the method and apparatus delineated above in section 2b(2)(c)(c2), entitled Automatic Insertion into a Ductus of an Inline Coupling Jacket-secured Bridging, or Tie-line and Its Replacement if Necessary, interruption in circulation, ischemia, reperfusion injury, the need for general anesthesia, and cardioplegia, which pose the potential for severe mid- and postprocedural complications in proportion to the duration of application, are eliminated.

The avoidance of these conventional measures through the use of the means described here eliminates as many if not more risks of complications than does the intrinsic procedure itself, and does so in proportion to the relative complexity and duration of the operation; where there is no cessation in perfusion, ischemia-reperfusion injury, as well as the other complications associated with the conventional means, are eliminated by definition.

By the same token, a complex defect of the aorta can originate at the aortic root, leaving no gap into which the end-connecting inline coupling jackets can be positioned, much less with an additional gap to accommodate a bridging over larger replacement once the original prosthesis has expanded with growth of the patient to its maximum.

2b(2)(e). Tie-Line Mounted Side-Entry Flow Diversion Jackets, Vascular Valves, and Servovalves

### Purposes for the Use of a Tie-Line Mounted Side-Entry Device

A tie-line mounted side-entry device is shown in FIG. 36G where the mounting is shown as in direct contact with its mounting inline coupling jackets to either side, but which could actually be farther apart from these than as shown in the drawing figure. Purposes for using a tie-line mounted side-entry device include:
1. To replace malacotic or otherwise unacceptable substrate vessel segment in order to mount a side-entry device.
2. To make possible the simultaneous mounting of multiple side-entry devices of the same or different kinds.
3. To avoid tissue-hyperreactive substrate vessel segment in lieu of a direct mounting.
4. To allows avoiding a structure or another disorder system component which would be interposed between the expected side-entry device mounting site by relocating the mounting site to a level along the substrate ductus up- or downstream to that usual.

### Inline Coupling Jacket-Secured Tie-Line Mounted Servovalves

A shown in FIG. 37A, a tie-line mounted side-entry device such as a vascular servovalve is mounted no differently than is an inline vascular prosthesis. Where multiple vascular servovalves are required as in a compound vascular bypass heart transplant, multiple tie-line mounted solenoid-driven valves or servomotor-driven servovalves can be fixed in position simultaneously to feed into and outlet the confluence conduits, which combination of resources significantly reduced the duration of the procedure.

Ordinarily, the structural features incorporated into valves such as a compliant, nonirritating lining, openings to the surrounding space, and druglines to deliver remedial medication, if necessary, should be sufficient to dispel valve-reactive complications. If not, then mounting the valve to a tie-line will eliminate direct contact between the valve and a substrate ductus. Use thus is reserved only when direct contact causes an intractable valve-reactive response such as swelling or stenosis that prevents proper valve function. FIG. 36G shows an inline coupling jacket-secured tie-line mounted servovalve.

Such a tie-line can be flexible, if used in a neonate, made of the expanding fabric described shortly below in section 2b(2)(f), entitled Expandable Structure to Accommodate Growth and Self-adjust in Size in Vascular Segment Prostheses, Lines, and Confluence Conduits Placed in Children, or if used in an adult can be a solid base-tube made of poly(aryl-etherethereketone), stainless steel, or titanium, for example. Even with the inline coupling jackets at either end, a tie-line with valve mounted to avoid contact with the vessel in order to avoid valve-reactive complications is relatively short and little extends beyond the upper and lower edges of the valve.

Introduced and secured at either end by inline coupling jackets, insertion of the tie-line or the base-tube in the ductus is without clamping or the need to interrupt the flow of blood. For this reason, insertion of a tie-line or base tube can be midoperative without ischemia and little risk of vasospasm. The mechanism that makes this possible is described in the section preceding this, 2b(2)(c)(c2), entitled Automatic Insertion into a Ductus of an Inline Coupling Jacket-secured Bridging, or Tie-line and Its Replacement if Necessary.

Mounting a vascular valve along a tie-line or base-tube which little extends beyond the up- and downstream edges of the valve itself can be used to overcome any valve-reactive degenerative process in the substrate ductus that mounted in direct perivascular relation to the substrate ductus would affect the internal diameter of the ductus to interfere with the proper functioning of the valve or induce a degenerative process that would impair the vessel.

Greater extension of the tie-line up- and/or downstream allows positioning the valve without encroachment on neighboring tissue or another component of the implanted disorder response system. This makes it possible to provide adequate clearance for a stent-jacket or extraction jacket, for example, described in U.S. Pat. No. 11,389,171, entitled Integrated System for the Infixion and Retrieval of Implants, and to keep valved vessels from relations of adjacency that would result in pinching, kinking, or abrasion by neighboring tissue or another component of the implanted disorder response system.

2b(2)(f). Expandable Structure to Accommodate Growth and Self-Adjust in Size in Vascular Segment Prostheses, Lines, and Confluence Conduits Placed in Children

Circulatory inadequacy is a major factor in maldevelopment. Placed in pediatric patients to replace defective vascular tissue, inflexible and nonexpandable bloodlines, druglines, tubular and anatomical tie-line type prostheses and confluence conduits would begin to induce constricted and aberrant circulation which becomes more anomalous in proportion to the growth which ensues since these were placed. This condition continues until the time-limited repair must be replaced, whereupon the patient will most often have to undergo another open chest procedure after which the process of losing circulatory sufficiency over time will resume, and the pain following a thoracic incision can persist for years.

Shown in FIGS. 15C, 15D, 36A, and 36F, the flats 233 and vaults 234 expandable structure made of translucent if not clear transparent polypropylene or polyetheretherketone to accommodate growth in a prenate, neonate, infant, or young toddler apply to all bloodlines, druglines, and confluence conduits. Resistant to deformation from a side, to optimize laminar or streamline flow and minimize any tendency to accumulate clot as would a saccular aneurysm, the vaults are kept relatively shallow with rounded edges, and if necessary, drugline 8 can be used to release a slow heparin drip.

FIGS. 15C and 15D show at the top an expandable confluence conduit 230 and at the bottom, a venous bloodline 196′ secured at either end by vascular valves, while FIG. 37A shows a nonexpandable tie-line type prosthetic bloodline for use in an adult secured at either end by inline coupling jackets as addressed above in section 2b(2)(c), entitled Vascular Segment Prostheses: Inline Coupling Jackets and Bridging, or Tie-lines, and its subordinate sections.

Beginning with intrauterine growth restriction as a cause of inadequate oxygenation and nutrition, circulation inadequacy is a key factor in maldevelopment. Much as expedient measures to repair a defective heart such as one univentricular rather than to replace the heart even though a competent graft organ is available, the conventional three stage surgical response usually prevents the baby from dying, but is inadequate for producing normal rather than nonpulsatile blood flow referred to as Fontan circulation addressed above in section 2b(1)(b)(b7), entitled Metered Compound Vascular Bypass, or Switched-organ Liver or Heart-Liver Transplantation, and therewith, maldevelopment that unless the defective circulation is remedied, will continue throughout life.

Maldevelopment due to inadequate circulation is also neurological, and the plasticity of nerve and the adaptability of organs and tissues that pertain following other causes for impaired development themselves inadequate, these children do poorly in school and have been rendered incapable of significant achievement. Furthermore, usually frail and cyanotic at the outset, these patients are neither prepared to undergo revisions following inadequate initial attempts at repair. The object following a period of halfway measures to fix a condition that jeopardizes the balance of life is to provide an initial repair that will fix the problem once and for all and exhibit unquestioned durability.

The harm initiated at the outset of life through the application of such halfway measures is the subject of section 2a(1) above, entitled Congenital Defects of the Heart and Other Organs—The Importance of Prompt Intervention, and is addressed in copending application Ser. No. 17/689,880, entitled Prosthetic Disorder Response Systems. In a neonate or infant, bloodlines, druglines, tubular and anatomical tie-line type prostheses and confluence conduits made to replace defective tissue along the central arterial or venous tree which does not expand in step with growth represents a critical impediment to normal development.

Not only does a growth-noncompliant line or conduit interfere with normal growth at the site of the patch or prosthesis itself, but as documented above in section 2b(2)(c), entitled Vascular Segment Prostheses: Inline Coupling Jackets and Bridging, or Tie-lines, in the subsection thereof entitled Improved Sufficiency of Seamless Prostheses Compared to Alternative Measures Such as Patches, Angioplasty, Hybrid Endovascular/Extravascular, and/or Stent Grafting, it prevents normal growth in neighboring tissue, often resulting, for example, in upper body hypertension and impaired development of the left arm.

In fact, this can interfere with the normal development of all the tissues and organs of the body. Neonates grow quickly, and a patch touted as high in tensile strength exhibits a mechanical property that is beneficial in an adult but injurious in proportion to the youthfulness of the patient-early in if not from the outset when growth and development proceed at the highest rate with the consequences for the rest of life most pronounced, locked in, and important. High tensile strength in a patch aortoplasty placed early in life only means that the restriction in growth and development it will cause commences at a time when these proceed most quickly to persist as irreversible.

All parts of expandable bloodlines, druglines, and tubular and anatomical tie-line type prostheses and confluence conduits must also comply with the increase in internal dimensions required to accommodate growth. Expandable components must be capable of durability for years if not to the end of life. Exceptionally, those addressed above in section 2b(1)(b)(b14), entitled Confluence Conduits: Connection of One or More Bloodlines to Flow through a Common Channel to the Same or a Different Number of Bloodlines with Self-leveling Apportionment of Outflow, in the closing subsection thereof entitled Tiny Vessel Grouped Inlet/Outlet Expandable Confluence Conduits and below in section 6, entitled Description of the Preferred Embodiments of the Invention, in the subsection thereof entitled Tiny Vessel Grouping Device, and shown in FIGS. 15C and 15D are intended for temporary use long enough to allow the patient to gain in strength. Expandable components other than lines or conduits are addressed below in section 6, entitled Description of the Preferred Embodiments of the Invention, in the opening subsection thereof entitled Vascular Valves and Servovalves in General.

Due to the myriad factors that affect a fetus, except for a specified cohort or population, consistency in normal growth data is so elusive as not to exist (see, for example, Zhonggui, X., Ping, Z., Jian, K., Feimin, S., and Zeyuan, X. 2022. “The Growth Rates and Influencing Factors of Preterm and Full-term Infants: A Birth Cohort Study,” Online, Medicine (Baltimore, Maryland) 101(34): e30262; Huang, L., Yang, S., Yang, F., and Xiong, F. 2019. “A Prospective Study about Physical Growth of Children from Birth to 2 Years Old Born Full-term Small-for-gestational-age,” Journal of Paediatrics and Child Health (Melbourne, Australia) 55(2): 199-204; Villar, J., Papageorghiou, A. T., Pang, R., Ohuma, E. O., Cheikh Ismail, L., and 13 others 2014. “The Likeness of Fetal Growth and Newborn Size across Non-isolated Populations in the INTERGROWTH-21st [International Fetal and Newbom Growth Consortium for the 21st Century] Project: The Fetal Growth Longitudinal Study and Newborn Cross-sectional Study,” Online, Lancet, Diabetes and Endocrinology (London, England) 2(10): 781-792; Ehsanpour, S., Hemmati, E., and Abdeyazdan, Z. 2012. “Comparison of Neonatal Growth in Normal, Low and Very Low Birth Weights until 18 Months,” Online, Iranian Journal of Nursing and Midwifery Research (Mumbai [Bombay], India) 17(2 Supplement 1): S131-S136; Johnson, W., Choh, A. C., Soloway, L. E., Czerwinski, S. A., Towne, B., and Demerath, E. W. 2012. “Eighty-year Trends in Infant Weight and Length Growth: The Fels Longitudinal Study,” Online, Journal of Pediatrics (St. Louis, Missouri) 160(5): 762-768; de Onis, M., Garza, C., Onyango, A. W., Rolland-Cachera, M. F., 2009. “WHO [world health organization] Growth Standards for Infants and Young Children,” (in French with English abstract at Pubmed) Archives de pediatrie (Paris, France) 16(1): 47-53; Fenn, B. and Penny, M. E. 2008. “Using the New World Health Organisation Growth Standards: Differences from 3 Countries [India, Peru, and Vietnam],” Journal of Pediatric Gastroenterology and Nutrition (Philadelphia, Pennsylvania) 46(3): 316-321; Pandove, S. P., Singh, K., and Sandhu, A. S. 1994. “Growth of Term Infants during Neonatal Period,” Indian Pediatrics (New Delhi, India) 31(6): 675-678).

Slightly larger in diameter than the substrate ductus, an inline coupling jacket-secured expandably structured prosthesis allows a defective segment along a ductus to be replaced without injurious protrusion into neighboring structures despite the compactness of the anatomy. Provided it has adequate transparency, a vascular segment replacement prosthesis made of polypropylene or polyetheretherketone, for example, expandable in an infant and nonexpandable in an adult, will allow visual confirmation of the relative proportion of blood flowing through the native and prosthesis segments during the replacement process described above in section 2b(2)(c)(c2), entitled Automatic Insertion into a Ductus of an Inline Coupling Jacket-secured Bridging, or Tie-line and Its Replacement if Necessary, and illustrated in FIG. 37A.

However, once placed, inline coupling jackets no longer provide this flow transfer valving function. Used where the native tissue is removed, function as flow diversion switches such as those solenoid-driven which allow flow to be switched between either of two lumina, or vascular servovalves which allow flow to be differentially apportioned over a selectable interval is lost. This notwithstanding, an ability to modulate the rate of flow through a segment along an artery such as the thoracic aorta would appear to offer little medical benefit.

In any practical situation, the greater importance of either a significant reduction in size, weight, projectedness, and the ability to accommodate growth or the capability to switch between either of two pathways will be clear. In choosing between an inline coupling jacket secured prosthesis or a side-entry flow diversion jacket-secured bypass, that flow rate control is not a factor means that the less obtrusive prosthesis should be preferred. While in the chest, the greater good goes to unobtrusiveness elsewhere, provided the native tissue is sound, meterability might confer an advantage.

Using the expandable structure delineated here, a vascular prosthesis such as a replacement thoracic aorta for placement in a child with a connective tissue disorder, a congenital aneurysm, and the probability of future sites of structural failure beyond the border of a patch aortoplasty, for example, prosthesis bridging, or tie-lines are meant for lifelong implantation. Nevertheless, if for any reason, the original prosthesis would require to be removed, a means for its replacement-again, without interruption in the flow of blood through the native vessel—is included in section 2b(2)(c)(c2) above entitled Automatic Insertion into a Ductus of an Inline Coupling Jacket-secured Bridging, or Tie-line and Its Replacement if Necessary.

Whether due to growth or self-adjustment when used as a bypass or shunt, the structure spontaneously expands in response to an increase in blood pressure. If a hypertensive episode occurs, a pair of springs returns the device to the size proper for normotension. Uses for the device include to self-adjust in both caliber and length when placed as 1. A shunt or bypass which because it spontaneously adjusts to the blood pressure can serve in lieu of a tuned shunt as addressed below in section 2b(2)(h)(h1), entitled The ‘Tuned’ Shunt, and its subsections and shown in FIG. 22F; 2. A vascular prosthesis; and 3. In response to the increase in blood pressure with growth.

When used as a prosthesis, the expandable structure is placed in the elevated position of the mechanism described above in section 2b(2)(c)(c2), entitled Automatic Insertion into a Ductus of an Inline Coupling Jacket-secured Bridging, or Tie-line and Its Replacement if Necessary, and depicted in FIGS. 37A and 37C thru 37E. The mechanism the transects the native segment as it rotates the prosthesis in position so that blood flow is gradually transferred from the native segment into the prosthesis.

If appropriate in a patient susceptible to deviations in blood pressure, the prosthesis is chosen to be slightly longer once fully expanded to accommodate an increase in blood pressure. This section will explain how once the patient commences taking blood pressure medicine or the condition spontaneously resolves, resumption of the prosthesis to the normotensive dimensions is spontaneously assumed. The value in vascular prostheses that would accommodate growth through adulthood is irrefutable. For example, a prosthetic thoracic aorta to replace one congenitally defective in a neonate or infant that would eliminate the need to reenter the chest for life is of such advantage that it must supplant every alternative means of repair.

With respect to vascular prostheses, the object is to provide a tubing material suitable for use in coupling jacket-secured bridging or tie-line vascular prostheses, usually to replace major arteries, as delineated above in sections 2b(2)(c), 2b(2)(c)(c1), 2b(2)(c)(c2), and 2b(2)(c)(c3), to include prostheses anatomically shaped such as a replacement thoracic aorta with supraaortic branches, which expandable up to adult size will spontaneously adjust in size to the existing dimensions of the patient whether adult or pediatric and thereafter continue to adapt to fit the patient regardless of an increase in size due to growth.

In such use, both the material and its configuration must be suitable, and measures must be provided to allow use in vessels less than 6 millimeters in diameter where clot has always been a problem. With the understanding that the operator adjusts the prosthesis, which is packaged at the its smallest, unexpanded, size for a neonate-such as a replacement aortic arch with supraaortic branches or the abdominal aorta to include the celiac trunk-so that it fits the segment to be removed, it is fair to say that one starting size of each distinct type prosthesis before the operator manually opens it to the size for the patient fits all.

The different prostheses comprise the thoracic aorta to just short of its ascent and descent to allow the space needed to apply the inline coupling jackets, and subsegments thereof to include the ascending aorta to just short of its ascent, the aortic arch with supraaortic branches, and the descending aorta, the abdominal aorta in its entirety, or its suprarenal abdominal or paravisceral segment, or its infrarenal segment, and straight vessel replacement line provided in a coil so the operator can cut the line to the length required. In infants, such line is also used for druglines that are to remain in place indefinitely.

### Consequences of Endothelial and Baroreceptor Deficits Following Removal for Placement of a Prosthesis

The potential consequence of endothelial removal to place a prosthesis depends upon the extent thereof, while vasopressor or baroreceptor removal results in deficits over a period during which compensatory sensation following placement of a prosthesis tends to gradually recover due to collateral stimulation, attributable to stimulus generalization and equivalence, primarily to vision and kinesthetic sensation. Immediate or localized endothelial function coextensive with the endothelium, and a synthetic prosthesis as such has no need for endothelial support, so that unless the tie-line type prosthesis covers a large area, little in the way of complications should ensue for the circulatory system as a whole due to the removal of that proportion of intima.

However, if the prosthesis is extensive, the wider circulatory system will likely be affected, so that additional medicinal support will be necessary. More specifically, when numerous endothelial cells and more particularly the aortic body have been lost, the promptness of response in adjustment of the blood pressure may be inadequate or missing of which one possible consequence may be that following an initial period of occasional syncope, the patient will adjust to the deficit. Generally, a loss of baroreception is gradually compensated for.

The literature offers relatively little as to the consequences for sustained sufficiency in the rest of the circulatory system due to a significant defect following its replacement with a prosthesis that required the removal of the innate baro- and chemoreceptors and a larger if localized amount of the endothelium, and conventional repairs offer no more than elasticity to accommodate an elevation in blood pressure, for example, if that (see, for example, DeRoo, E., Stranz, A., Yang, H., Hsieh, M, Se, C., and Zhou, T. 2022, Op cit., above in this section; Wu, W., Zhang, J. Shao, L., Huang, H., Meng, Q., Shen, Z., and Teng, X. 2022. “Evaluation of Circulating Endothelial Progenitor Cells in Abdominal Aortic Aneurysms after Endovascular Aneurysm Repair,” Online, International Journal of Stem Cells [Seoul, South Korea] 15(2): 136-143; Sun, J., Deng, H., Zhou, Z., Xiong, X., and Gao, L. 2018. “Endothelium as a Potential Target for Treatment of Abdominal Aortic Aneurysm” Online, Oxidative Medicine and Cellular Longevity (New York, New York) 2018:6306542). Any of these substances in fluid form can be released by the implanted disorder system.

If refractory or combined with an impaired sense of balance in an elderly patient, for example, a measure of blood pressure control can be simulated through the placement of thin film piezoelectric pressure sensors and the automatic release of nitrates, fludrocortisone, midodrine, pyridostigmine, fludrocortisone, caffeine, epinephrine, or norepinephrine bitartrate, for example, to elevate the pressure. Through the immediate detection of a need for adjustment in an autonomic function, a fully implanted prosthetic disorder response system can directly pipe-target a potent drug to counteract the malfunction to the affected organ, organs, or the systemic circulation, and such response can support or compensate for an inadequacy in innate function of any kind whether a blood, immune, or digestive dysfunction or disease.

Despite the facts that the aorta is the principal artery which delivers blood to the rest of the circulatory system and that an aortic aneurysm can develop from upstream to the descending aorta to downstream behind the thoracic diaphragm through the aortic hiatus between the left and right crus and abdomen thence into the iliac bifurcation, thence at the knee in the popliteal vessels, an innate mechanism that would spontaneously compensate for a major loss in secretory endothelial surface area would simply be lacking or appear or limited to an overreaction in endocrine response.

Aneurysmal counteractive drugs specified above in this section, the direct pipe-targeting of such medication into an incipient or emergent aneurysm once detected can be programmed to initiate stem cell-based therapy to retard if not stop the continued development of the defect Jung, H., Youn, D. H., Park, J. J., and Jeon, J. P. 2023. “Bone-Marrow-derived Mesenchymal Stem Cells Attenuate Behavioral and Cognitive Dysfunction after Subarachnoid Hemorrhage via HMGB1-RAGE [high-mobility-group box protein 1-receptor for advanced glycation end products] Axis Mediation,” Online, MDPI [Multidisciplinary Digital Publishing Institute] Life (Basel, Switzerland) 13(4): 881; (DeRoo, E., Stranz, A., Yang. H., Hhsieh, M, Se, C., and Zhou T. 2022, Op cit. above in this section; Khan, A., Bellio, M. A., Schulman, I. H., Levi, A. D., Longsomboon, B., and 17 others 2021. “The Interdisciplinary Stem Cell Institute's Use of Food and Drug Administration-expanded Access Guidelines to Provide Experimental Cell Therapy to Patients with Rare Serious Diseases,” Online, Frontiers in Cell and Developmental Biology (Lausanne, Switzerland) 9:675738; Brunet, M. C., Chen, S. H., Khandelwal, P., Hare J. M., Starke, R. M., Peterson, E. C., and Yavagal, D. R. 2019. “Intravenous Stem Cell Therapy for High-grade Aneurysmal Subarachnoid Hemorrhage: Case Report and Literature Review,” World Neurosurgery (New York, New York) 128:573-575; Ghonim, H. T., Shah, S. S., Thompson J. W., Ambekar, S., Peterson, E. C., and Elhammady, M. S. 2016. “Stem Cells as a Potential Adjunctive Therapy in Aneurysmal Subarachnoid Hemorrhage,” Online, Journal of Vascular and Interventional Neurology (Minneapolis, Minnesota) 8(5): 30-37; Siasos, G., Mourouzis, K. Oikonomou, E. Tsalamandris, S. Tsigkou, V., and 9 others 2015. “The Role of Endothelial Dysfunction in Aortic Aneurysms,” Current Pharmaceutical Design [Bentham, Dubai, United Arab Emirates] 21(28): 4016-4034). This view warrants reconciliation with another which would appear inconsistent with it (Perissiou, M., Bailey, T. G., Saynor, Z. L., Shepherd, Al., Harwood, A. E., and Askew, C. D. 2022. “The Physiological and Clinical Importance of Cardiorespiratory Fitness in People with Abdominal Aortic Aneurysm,” Online, Experimental Physiology 107(4): 283-298).

If the extent of an aortic prosthesis is sufficient to warrant the replacement of endothelial function and autonomic function, or the length of an arterial prosthesis is sufficient to warrant the replacement of endothelial function, this is provided through the direct pipe-targeting to the prosthesis of the appropriate agents by the implanted prosthetic disorder response system. To fit any patient, prostheses other than plain line are made to expand up to five times the starting dimensions.

The circumferential vaults, monolithic with the rest of the prosthesis as seamless, are inelastic and do not stretch to allow reaching the adult caliber. These are therefore provided in the adult size ab initio. This in contrast to the flats which are unrestricted in length or number. To prevent the vaults from projecting outward and irritating neighboring tissue in a child, the prostheses are dispensed with the vaults half-way inverted so that their silicone rubber coated top presents a furrow between parabolic or ogive elevations running along the sides that present a nonprotrusive surface, or if not fully half-inverted, a running double row of elevation to either side of the center depression which silicone rubber coated, are well cushioned.

Half-way inversion is to prevent the apices of the vaults protruding into the lumen. The disruption in laminar, or streamline, blood flow caused by passage of the vaults until fully extended should not be more pronounced than that produced by the passage of blood flow past the ostia of branches, and if problematic is easily resolved by having the automatic implanted disorder response system initiate an intermittent heparin drip to dispel any accumulation of clot.

As a generalization, with conventional prostheses—in particular when the innate sensors, the baro- and chemoreceptors in the carotids and aorta had been removed along with the defective tissue and replaced with a synthetic prosthesis—the clinician can only reinstate spontaneous endothelial and endocrine-mediated function through the use of medication. If the condition is intermittent or episodic, there is no immediacy in synchronization between the altered level of the physiological function and the need for the medication. Tie-line type vascular prostheses are superior to conventional patches, stent-grafts, and other types of vascular repairs in allowing the direct targeting to the area of repair of substances to compensate for and offset a loss in innate baro- and chemoreceptive sensation and support healing at the site.

Providing one or more druglines, or accessory channels, also makes it possible to directly pipe-target medication to the prosthesis to counteract an adverse foreign body reaction, infection, or almost any other complication. Targeting thus is possible because the implant incorporates a drugline, or accessory channel attached to one or more small drug reservoirs usually implanted subcutaneously in the pectoral region. The prosthesis itself having no need for drugs, the release of these into the prosthesis through its inlet inline coupling jacket accessory channel-engaged drugline, part number 8 in FIG. 36A, can become desirable when the prosthesis reaches the threshold extent of coverage associated with an amount of endothelium and/or aortic chemo- and baroreceptor removal as could affect the circulatory system as a whole.

A relatively simple microcontroller-administered prosthetic disorder response system can be programmed to release normotensive-deviant blood pressure-counteractive medication, for example, into the prosthesis. Responsive to a sensor-reported increase in activity level which would generate baroreceptor and endothelial output had these remained intact rather than replaced by a prosthesis, the system releases medication to simulate the lost function. Such inputs fed to the implanted prosthetic disorder response system causes it to actuate a drug reservoir outlet pump to release vasodilatory or vasoconstrictive medication as appropriate.

Fully extended for placement in an adult, the vaults 234 are level with the flats 233. The structure of the prosthesis is designed to accommodate the endoluminal blood pressure to which it expands, so that absent any supplementation, it is intrinsically irreversible. For this reason, to impart the device with contractility so that it will recede to the proper size upon the resolution of an increase in blood pressure, small gold plated or parylene coated titanium expansion springs which are completely compressed at the proper blood pressure can be easily hooked to small projections at the top and bottom of the prosthesis.

The object to provide prostheses that can be implanted once and left in place indefinitely precludes the use of springs in other than adults. As specified above in section 2a, entitled Background of the Invention. General, in the subsection entitled Adaptation of the Expandable Structure for Elevated Blood Pressure, the use of springs thus is suitable only in a patient who is fully grown and whose hypertension is temporary or intermittent. Otherwise, the undersized springs will prohibit the expansion of the prosthesis to the proper dimensions. In cases of both neonatal hypertension and a serious defect such as a larger coarctation or an interrupted aortic arch where the cause is a congenital connective tissue disorder, the thoracic aorta should be replaced with a prosthesis of the kind described.

Should the preferred structure be prevented from production due to a problem of supply, for example, another material which can satisfy the requirements for an expandable prosthesis is a suitably plasticized parylene sputter coated polyurethane laminate sheet embedding longitudinally oriented 50-pound interwoven multistrand para-aramid (poly-paraphenylene terephthalamide K119) or polyamide-imide Kevlar® (E.I. DuPont de Nemours and Company, Inc., Wilmington, Delaware) cord. The nonelastic sheet is circumferentially pleated under heat in an hydraulic press.

Allowed to return to room temperature, the pleated polyurethane laminate sheet with embedded cords is tubulated, wrapped around a mandrel core with pleated surface, and placed within an injection mold having an internal surface pleated to match that of the tubulated sheet. The sheet is then invested within an adherent surrounding seamless nonelastic plasticized synthetic rubber mold-added tube of a parylene coated artificial polyisoprene or a single backed closed-cell foam neoprene (polychloroprene), or nonallergenic nitrile rubber, for example. For use in an adult the device is manually expanded to the length needed and small springs used to accommodate hypertension as explained below for the preferred expansion structure.

A constrictive defect being the probable cause, its removal is likely to prove sufficient treatment to dispel the hypertension. If not, then treatment of the hypertension is conventional such as the administration of propranolol or hydralazine, or in severe cases, intravenous nicardipine, enalaprilat, or hydralazine, for example (Hassan, R. and Verma, R. P. 2022. “Neonatal Hypertension,” Online, Treasure Island, Florida: StatPearls Publishing Co; Flynn, J. T. 2020. The Hypertensive Neonate,” Seminars in Fetal and Neonatal Medicine 25(5): 101138; Starr, M. C. and Flynn, J. T. 2019. “Neonatal Hypertension: Cases, Causes, and Clinical Approach,” Online, Pediatric Nephrology (Berlin, Germany) 34(5): 787-799, erratum 34(9): 1637; Deliu, A. G., Saneerappa, P. B. J., Franklin, O., and Letshwiti, J. 2018. “Sodium Nitroprusside, a Lifesaving Treatment for Neonatal Hypertension: An Irish Experience,” Online, British Medical Journal Case Reports (London, England) 2018: bcr2017221856; Nickavar, A. and Assadi, F. 2014. “Managing Hypertension in the Newborn Infants,” Online, International Journal of Preventive Medicine (Isfahan, Iran) 5 (Supplement 1): S39-S43).

The diameter of the normal thoracic aorta decreases by tapering in the downstream direction, but in later preterm neonates it is about 0.6 centimeters at the upstream, or larger end, while in larger neonates, it is about 1.0 centimeter (see, for example, Abushaban, L., Mariappa, T. V., Rathinasamy, J., and Sharma, P. N. 2018. “Normal Reference Ranges for Aortic Diameters in Preterm Infants,” Online, Journal of the Saudi Heart Association (Riyadh, Saudi Arabia) 30(2): 86-94; Walther, F. J., Siassi, B., King, J., and Wu, P. Y. 1985. “Normal Values of Aortic Root Measurements in Neonates,” Pediatric Cardiology 6(2): 61-63).

In a larger adult, the diameter of the ascending aorta toward its origin can be as large as 4.5 centimeters without arising concern (see, for example, Patel H N, Miyoshi T, Addetia K, Citro R, Daimon M, and 22 others 2022. “Normal Values of Aortic Root Size According to Age, Sex, and Race: Results of the World Alliance of Societies of Echocardiography Study,” Online, Journal of the American Society of Echocardiography (St. Louis, Missouri) 35(3): 267-274; Saliba, E. and Sia, Y; 2015. “The Ascending Aortic Aneurysm: When to Intervene?,” Online, International Journal of Cardiology, Heart and Vasculature (Shannon, Ireland) 6:91-100; Mao, S. S., Ahmadi, N., Shah, B., Beckmann, D., Chen, A., and 4 others 2008. “Normal Thoracic Aorta Diameter on Cardiac Computed Tomography in Healthy Asymptomatic Adults: Impact of Age and Gender,” Online, Academic Radiology (Reston, Virginia) 15(7): 827-834).

Congenital defects may be associated with an anomalous diameter of the aorta both before and following repair (see, for example, Putotto, C., Pugnaloni, F., Unolt, M., Maiolo, S., Trezzi, M., and 6 others 2022. “22q11.2 Deletion Syndrome: Impact of Genetics in the Treatment of Conotruncal Heart Defects,” Online, MDPI [Multidisciplinary Digital Publishing Institute] Children (Basel, Switzerland) 9(6): 772; Putotto, C., Pulvirenti, F., Pugnaloni, F., Isufi, I., Unolt, M., and 8 others 2022. “Clinical Risk Factors for Aortic Root Dilation in Patients with 22q11.2 Deletion Syndrome: A Longitudinal Single-center Study, Online, MDPI [Multidisciplinary Digital Publishing Institute] Genes (Basel, Switzerland) 13(12): 2334;

Chang H W, Kim S H, Hakim A R, Chung S, Kim D J, and 4 others 2020. “Diameter and Growth Rate of the Thoracic Aorta-Analysis Based on Serial Computed Tomography Scans,” Online, Journal of Thoracic Disease (Hong Kong, China) 12(8): 4002-4013; Cruz, C., Pinho. T., Sousa, C., Dias, C. C., Silva Cardoso, J., and Maciel, M. J. 2018. “Ascending Aorta in Tetralogy of Fallot: Beyond Echocardiographic Dimensions,” Echocardiography (Hoboken, New Jersey) 35(9): 1362-1369; Cruz, C., Pinho, T., Ribeiro, V., Dias, C. C., Silva Cardoso, J., and Maciel, M. J. 2018. “Aortic Dilatation after Tetralogy of Fallot Repair: A Ghost from the Past or a Problem in the Future?, (in English and Portuguese) Revista Portuguesa de Cardiologia (English edition) 37(7): 549-557; Niwa, K. 2018. “Aortic Dilatation in Complex Congenital Heart Disease,” Online, Cardiovascular Diagnosis and Therapy (Sheung Wan, Hong Kong, China) 8(6): 725-738; Niwa, K. 2005. “Aortic Root Dilatation in Tetralogy of Fallot Long-term after Repair-Histology of the Aorta in Tetralogy of Fallot: Evidence of Intrinsic Aortopathy,” International Journal of Cardiology (Amsterdam, North Holland) 103(2): 117-119).

To cover the growth of the thoracic aorta over the course of life calls for a generally fivefold increase in internal diameter, or caliber, and length. Use in conjunction with a fully implanted prosthetic disorder response system makes the use of an intermittent heparin drip effective for overcoming this obstacle, and making the conduit of resorption-unsusceptible polyetheretherketone of polypropylene rather than a polyester-family polymer.

Shown in FIGS. 15C, 15D, 36A, and 36F so that the circumferential expansion vaults will not irritate adjoining tissue or interfere with the adjacent positioning in the automatic insertion mechanism shown in FIG. 37A, the material is highly plasticized, allowing the circumferential expansion vaults to partially flex into the lumen, that is, partially invert, upon insertion in a pediatric patient in whom the device is smallest in size. Potentially nocuous, any residue of plasticizer is completely washed away prior to dispensing. Due to the high variability in origins and branches of the supraaortic arteries, a standardized embodiment can be supplemented with one that allows snap together segments.

Alternatively, made as a laminate, the polypropylene or polyetheretherketone outer and thicker articulated layer is lined with a biaxially oriented thin polyethylene terephthalate film which pliant but not stretchable projects through openings to form the vaults which fill outward through the outer layer, and to ensure durability against the harsh internal environment as well as to take advantage of its suppression of thrombus and intimal hyperplasia, made either way, both external and internal surfaces are chemical vapor deposition coated with parylene (Hao, D., Lin, J., Liu, R., Pivetti, C., Yamashiro, K., and 9 others 2023, Op cit., above early in section 2a, entitled Background of the Invention. General). Protheses made with the expandable structure described above in section 2b(2)(f), entitled Expandable Structure to Accommodate Growth and Self-adjust in Size in Vascular Segment Prostheses, Lines, and Confluence Conduits Placed in Children, are intended to make possible the replacement of great vessels, to include almost all of the thoracic aorta with the supraaortic branches without the need to interrupt circulation.

A congenital aneurysm, dissection, coarctation, or interruption in the thoracic aorta, for example, demands repair at birth, and to eliminate the need for later revision, an expandability that significantly exceeds that of Dacron®. In replacing an extensive portion of a vascular structure such as the thoracic aorta without the need to interrupt the flow of blood, cardiopulmonary support, or general anesthesia, the numerous risks associated with these also eliminated. As shown in FIG. 37D, stitching the inline coupling jackets to the stumps of the substrate vessel precludes migration, and dehiscible seams, responsible for many if not most adverse sequelae with conventional patches, have been eliminated.

The injection molded polyetheretherketone or polypropylene polymers specified for making anatomically and simple cylindrical tie-lines with the alternating flats and circumferential expansion vault pattern for placement in pediatric patients is recommended on the basis of their nonresorbability compared to polyester-family polymers. However, over a period of years, these polymers are known to be degraded by soil-borne bacteria, and the internal microbiome is sufficiently diverse for concern. Polyetheretherketone demands high temperatures for injection molding; however, unless their reference is precisely to the need for high temperatures, it does not, as claimed by Mbogori, et al., lack thermoformability (Mbogori, M., Vaish, A., Vaishaya, R., Haleem, A., and Javaid, M. 2022. “Poly-Ether-Ether-Ketone (PEEK) in Orthopaedic Practice—A Current Concept Review,” Online, Journal of Orthopaedic Reports 1(1): 3-7).

Biofilm formation and bacterial breakdown of polypropylene may not be averted, but the unrestrained use of antibiotics is to be avoided, and bacterial degradation is slow making its durability a problem in a landfill, but it is not so slow that if implanted in an infant, the adverse effect would not transpire by age 80, for example (Ali, S., Bukhari, D. A, and Rehman, A. 2023. “Call for Biotechnological Approach to Degrade Plastic in the Era of COVID-19 Pandemic,” Online, Saudi Journal of Biological Sciences (Riyadh, Saudi Arabia) 3093): 103583; Ru, J., Huo, Y., and Yang, Y. 2020. “Microbial Degradation and Valorization of Plastic Wastes,” Online, Frontiers in Microbiology (Lausanne, Switzerland) 21(11): 442; Vestby, L. K., Gronseth, T., Simm, R., and Nesse, L. L. 2020. “Bacterial Biofilm and Its Role in the Pathogenesis of Disease,” Online, MDPI [Multidisciplinary Digital Publishing Institute] Antibiotics (Basel, Switzerland) 9(2): 59).

A more immediate problem is susceptibility to clot (Kratz, A., Stanganelli, N., and Van Cott, E. M. 2006. “A Comparison of Glass and Plastic Blood Collection Tubes for Routine and Specialized Coagulation Assays: A Comprehensive Study,” Online, Archives of Pathology Laboratory Medicine 130(1): 39-44; Krag, C., Larsen, J., and Reumert, T. 1981. “Handling Properties and Thrombogenicity of Microsutures,” Journal of Microsurgery 2(3): 202-205; Dahlke, H., Dociu, N. and Thurau, K. 1980. “Thrombogenicity of Different Suture Materials as Revealed by Scanning Electron Microscopy,” Journal of Biomedical Materials Research 14(3): 251-268). Polypropylene is also the least thrombogenic polymer (Dart, A. J. and Dart, C. M.2011. “Biomaterials and Clinical Use,” volume 6, section 6.636.3.2.5, Nonabsorbable Suture Material, in Ducheyne, P. (ed.), Comprehensive Biomaterials Amsterdam, North Holland, Netherlands: Elsevier, Science Direct).

To the extent that polypropylene and polyetheretherketone lack intrinsic antimicrobial and/or anticlot repulsion, coating to satisfy these needs can be applied to the outer surface as a protective coating of suitable counteractants and occasionally released into the lumen from implanted drug reservoirs of the implanted prosthetic disorder response system. To this end, the flats and circumferential expansion vault configuration that increases in dimensions not by stretching but rather by opening provides a continuous surface suitable for sputter coating, for example, so that when extended, there is no splitting apart or fissuring of the otherwise continuous coating.

To be made in the 2.0 millimeter thick nonwoven single-ply polyetheretherketone or polypropylene conformation shown in FIGS. 15C, 15D, 36A, and 36F, the prostheses and tie-lines with circumferential flats and vaults are not extruded but injection molded with inserts as necessary to incorporate branches. Sufficient pliancy may necessitate the addition of a plasticizer which must be completely washed away prior to use. The apices of the interrupted circumferential expansion vaults are rounded to avoid irritating neighboring tissue. Used in moderation, the accumulated systemic dose of an implanted disorder response system instituted heparin drip to prevent clot in small caliber bloodlines should never rise so as to induce thrombocytopenia.

Nevertheless, to avert this contingency, the system can be enabled to sense a lack of platelet sufficiency and reverse this outcome through the release of folate, lithium carbonate, and/or corticosteroids, for example. A monolithic prosthesis of this kind presents no seams to its sides and includes this tissue eliminating it from a propagation of aneurysmal breakdown, where this tissue would otherwise be no less subject to failure whether as a secondary result of the repair or due to an underlying congenital connective tissue disorder which caused the original defect.

To reduce if not eliminate the need for revision due to growth, a novel extremely expandable fabric, high in tensile strength, with the compliance needed to accommodate the pulse, and unfoldable to increase both in circumference and length in the largest adult is provided. And to avoid the need for painful revision or reentry for any purpose, the fabric eliminates seams that could dehisce, and unlike existing materials is not woven, cannot leak, and is oblivious to hydrolytic or enzymatic breakdown.

Expansion compliant with growth is critically beneficial for the young with congenitally defective vessels which would otherwise bode considerable misery and the constant risk of rupture and death. Due in part to its monolithic structure and automated placement as delineated above in section 2b(2)(c)(c2), entitled Automatic Insertion into a Ductus of an Inline Coupling Jacket-secured Bridging, or Tie-line and Its Replacement if Necessary, placement is without interruption in the flow of blood, without the need for cardiopulmonary support or general anesthesia, and significantly less demanding in terms of procedural time to place.

That a major application for this type material is in major arterial prostheses for use in children where it eliminates the need for later revision, does not mean that this material is not less equally valuable for use in mainlines (bloodlines) and sidelines (druglines) which similarly must expand to the existing size of the patient in order to eliminate the need for replacement. As special measures would be required to dispel the susceptibility of conventionally used materials such as polyester-based patch materials to clot for use in conduits smaller than 6 millimeters in diameter (see, for example, Ravi, S. and Chaikof, E. L. 2010, Op cit., above in this section), these materials have been discounted.

For lifetime implantation, the material used must retain its chemical and structural integrity, its mechanical properties, not be resorbed even over decades, not leak or attract clot, and must be minimally if at all provocative of adverse tissue reactions. Some materials are excellent in the short term but degrade to release nocuous breakdown products. Implant materials that are subject to enzymatic and/or hydrolytic breakdown or dissolution cannot be used unless reformulated or somehow coated or encapsulated to prevent exposure to the internal milieu (see, for example, Darie-Nita, R. N, Rapa, M., and Frackowiak, S. 2022. “Special Features of Polyester-based Materials for Medical Applications,” Online, MDPI [Multidisciplinary Digital Publishing Institute] Polymers (Basel, Switzerland) 14(5): 951; Bizuidenhout, D. and Zilla, P. 2001. “Vascular Surgery: Biomaterials,” in Jürgen Buschow, K. H., Cahn R. W., Flemings, M. C., Ilschner, B., Kramer, E. J., Mahajan, S. and Veyssiere, P., Encyclopedia of Materials: Science and Technology, pages 9513-9518; Uretsky, G., Appelbaum, Y., Younes H., Udassin, R., Nataf, P., and 4 others 1990. “Long-term Evaluation of a New Selectively Biodegradable Vascular Graft Coated with Polyethylene Oxide-Polylactic Acid for Right Ventricular conduit. An Experimental Study,” Journal of Thoracic and Cardiovascular Surgery 10095): 769-776).

Subject to resorption and degradation, implantation for lifelong as opposed to an expedient, half-way measure, polyester family-based materials such as high-density polyethylene and polyethylene terephthalate, for example, are unsuitable (see, for example, Wang, W., Zhou, Z., Liu, N., Zhang, X., Zhou, H., and 3 others 2022. “Improving Biocompatibility of Polyester Fabrics through Polyurethane/Gelatin Complex Coating for Potential Vascular Application,” Online, MDPI [Multidisciplinary Digital Publishing Institute] Polymers (Basel, Switzerland) 14(5): 989; Ravi, S. and Chaikof, E. L. 2010. “Biomaterials for Vascular Tissue Engineering, Online, Regenerative Medicine (London, England) 5(1): 107-120).

More specifically, for lifelong use, these materials would have to be externally and as an especially costly factor, internally sputter or vapor deposit coated with parylene or gold. To apply such treatment to the interior wall of a line 6 or fewer millimeters in diameter would pose a significant problem. Polyetheretherketone (PEEK) and polypropylene do not pose a problem of resorption, making these clearly preferable. As to its configuration, the flats-alternated circumferential expansion vault formation shown in FIGS. 15C, 15D, 36A, and 36F, provides expansion in step with the increase in internal pressure associated with growth not by stretching with increasing pull back but rather by unfolding.

That is, expansion responsive to growth results from the increase in pressure against the flats which drives these outwards, opening the circumferential expansion vaults, and in so doing, expanding the lumen both in diameter and length. Citation of large vessel prostheses is not meant to discount the use of this fabric in bloodlines, druglines, confluence conduits, tie-lines, shunts, and bypasses in neonates and infants where the object is to avoid replacement to the end of life. The fabric is continuous from end to end and molded in a single piece, preferably of a single polymer such as thin polyetheretherketone. Repair, or here, replacement, is most often needed for the thoracic and/or abdominal aorta, which large and subject to relatively high pressure are more prone to failure. The range of expansion is meant to accommodate any patient from an infant to an adult. Placed in an adult, the prosthesis spontaneously expands to the size needed.

Compliance with the pulse responds to the blood pressure against the flats. Elasticity such as exhibited by spandex (Elastane®, Lycra®) polyester-polyurethane, or polyether-polyurea, copolymer fabric that increases in resistance to continued stretching as to progressively pose greater resistance to the pulse as the blood pressure increases until if continued, it will eventually reach its elastic limit as to least comply with the pulse.

The expansion structure described eliminates the kinking and laminar separation associated with the material currently in use (see, for example, Baldwin, C., Pandey, J., and Olarewaju, O. 2022. “Hemolytic Anemia,” Online, Treasure Island, Florida: StatPearls Publishing Co.; Tamura, K., Tatsuishi, W., Konishi, Y., Konno, N., Kato, Y., and Abe, T. 2022. “Hemolytic Anemia Caused by Kinked Graft 6 Months after Aortic Dissection Repair,” (BioMed Central) Journal of Cardiothoracic Surgery (London, England) 7(1): 308; Sabzi, F., Ghasemi, F., and Asadmobini, A. 2016. “Hemolytic Anemia Caused by Kinking of Dacron Grafts Implanted in Repair of after Aortic Dissection,” Online, Ethiopian Journal of Health Sciences (Jimma, Ethiopia) 26(5): 497-502).

The material conventionally used in vascular prostheses is woven polyethylene terephthalate sold as Dacron®, which even crimped or pleated will provide limited expandability with increasing constraint, but not from infancy to adulthood, and poses problems related to dehiscence and leaks even in adults, much less infants where growth without the need for reentry and revision is sought (see, for example, Nazer, R. I., Alhothali, A., and Albarrati, A. 2023. “In Vitro Modelling of Crimped Dacron Vascular Grafts for Aortic Root Replacement,” Online, American Journal of Cardiovascular Disease (Madison, Wisconsin) 13(2): 59-67; Hori, D., Kusadokoro, S., Shimizu, T., Kimura, N., and Yamaguchi, A. 2020. “Prosthetic Graft Dilation at the Aortic Arch in the Era of Hybrid Aortic Surgery,” Annals of Vascular Diseases (Tokyo, Japan) 13(2): 163-169; Aurigemma, D., Borquez. A., Lee, J., Newbury, R., Moore. J. W., and 3 others 2018. “Non- anastomotic Failure of Woven Dacron Tube Grafts in the Thoracic Aorta in Young Adults,” Journal of Cardiac Surgery (Hoboken, New Jersey) 33(10): 653-657; Singh, C., Wong, C. S., and Wang, X. 2015. ‘Medical Textiles as Vascular Implants and Their Success to Mimic Natural Arteries,” Online, MDPI [Multidisciplinary Digital Publishing Institute] International Journal of Functional Biomaterials (Basel, Switzerland) 6(3): 500-525 (previously cited above in section 2a. Background of the Invention. General); Etz, C. D., Homann, T., Silovitz, D., Bodian, C. A., Luehr, M., and 3 others 2007. “Vascular Graft Replacement of the Ascending and Descending Aorta: Do Dacron Grafts Grow?,” Annals of Thoracic Surgery (Amsterdam, North Holland, Netherlands) 84(4): 1206-1213; Robicsek, F. 2000. “The Growth of Crimped Dacron Aortic Bypass Graft: A Case Report,” International Journal of Angiology (New York, New York) 9(1): 18-19).

Moreover, multiply unfoldable to increase both in circumference and length, the fabric devised to accommodate growth and avert the need for replacement that would necessitate another painful open chest operation at the same time reduces the cost of production in that two if not one embodiment can accommodate patients of almost any size. Further to accommodate a wide range of patient sizes as well as growth, not only a prosthesis to replace a defective thoracic aorta for example, but synthetic vessels, confluence conduits, druglines, shunts, and bypasses can be made of the same fabric.

Complications avoided by using a prosthesis, or tie-line, made of the expandable fabric to include perigraft seroma and adhesions are addressed above in section 2a, entitled, Background of the Invention. General. Self-adapting in dimensions in response to the internal pressure, vascular prostheses and shunts described herein, to include replacement vessels, confluence conduits, tie-lines, and aneurysm, anastomosis, and carotid endarterectomy bypasses, are best made of this fabric for use in patients of any age.

While eliminating the need for painful revision in the chest, the same structure allows the use of one size prosthesis to fit an adult. To eliminate the need for recovery, the device must be invulnerable to dissolution, biofilm, and thrombus. Pure gold and rhodium impractical for use in intracorporeal prostheses, no one material remains that would remain indifferent to the hydrolytic and enzymatic factors that would degrade and foul it. A practical solution consists of the choice in materials, surface treatment, and the automatic dispensing of counteractants.

To eliminate sears or anastomoses such as those seen in patch aortoplasties and elephant trunk and frozen elephant trunk repairs, and therewith, the opportunity for dehiscence and leaks to develop, the replacement segment is extended beyond the levels within which a postoperative defect such as another aneurysm or dissection would be more likely to arise (see, for example, Di Marco, L., Pantaleo, A, Leone, A., Murana, G., Di Bartolomeo, R., and Pacini, D. 2017. “The Frozen Elephant Trunk Technique: European Association for Cardio-thoracic Surgery Position and Bologna Experience,” Online, Korean Journal of Thoracic and Cardiovascular Surgery 50(1): 1-7). With connective tissue disorders, this can justify replacement of almost all of the aorta.

Unaffected, the thoracic aorta is continuous, without anastomoses or abrupt changes in elastic modulus or coefficient of elasticity from one section to another along borders separating distinct areas such as the native tissue and a polyethylene terephthalate patch. Distinct in reacting to stretching forces such as posed by the pulse and growth in the surrounding tissue, especially growth exceeding the ability of the synthetic fabric to expand, where such forces are strongest, the seams along the outer edges of such a patch are susceptible to dehisce where if not repaired promptly, a leak can be fatal.

Defects of the thoracic aorta congenital, a special fabric to replace a segment along a major vessel of a neonate with a genetic defect such as a connective tissue disorder which could lead to another aneurysm along the aorta at any time is addressed above in sections 2a, Background, General, and 2b(2)(f), entitled Expandable Structure to Accommodate Growth and Self-adjust in Size in Vascular Segment Prostheses, Lines, and Confluence Conduits Placed in Children.

No current method for the repair of such a defect adequate, this expansion structure can be incorporated into a prosthesis to replace the entire thoracic, thoracoabdominal, and abdominal aorta. To do so in a single procedure would require several assistants to manage the numerous branches, but the relatively long procedure, free of life support, and more generally, with the cessation of circulation through the affected segment, organ, or volume of tissue, general anesthesia, or cardioplegia would be safer and with a sedative such as ketamine used, relatively free from complications.

As shown in FIG. 36F, such a prosthesis can be anatomically shaped, or to facilitate placement, adjusted in caliber along its length to closely approximate the normal rate of flow-through the successive segments. Because a patch aortoplasty will not proportionally expand to accommodate growth with the rest of the thoracic aorta, a severe congenital malformity along the ascending aorta, for example, is viewed as justifying replacement of the entire thoracic aorta. From a preventive standpoint, such may presuppose a congenital susceptibility to aneurysmal failure elsewhere along the thoracic aorta justifying its replacement in whole for that reason as well as because a unit replacement will expand over the years without necessitating reentry.

Such a prosthesis overcomes the shortcomings of current methods of repair, is elastic to allow considerable expansion for growth and so avoid the need for revisions in a baby already impaired, and significantly, is emplaced automatically by the implanted prosthetic disorder response system, which accomplishes the replacement in a single action that rotates the tie-line into the position occupied by the native vessel in one continuous action that avoids the need to interrupt the flow of blood through the affected segment of which the consequence is likely to be inferior and less durable outcome. The special fabric described here and in section 6 below entitled Description of the Preferred Embodiments of the Invention, should expand sufficiently to allow the need for reentry following placement in an infant not until adolescence in a large patient and never in one not especially large.

### Alternating Circumferential Flats 233 and Vaults 234 Structure

1. The outer casing of expandable coupling valve-jacket-connected bridging, or tie-lines, described below in section 2b(2)(c)(c3), entitled Types of Inline Coupling jacket-Secured Bridging, of Tie-lines;
2. The casing of any jacket which must accommodate growth and/or the pulse; and
3. The shorter of the two confluence conduits to be implanted in a prenate, neonate, or small child, addressed below in section 2b(1)(b)(b15), entitled Switchable Lines and Confluence Conduits: Long when Intercorporeal, to Short when Intracorporeal, Both Affording Connection of One or More Bloodlines to the Same or a Different Number of Bloodlines with Self-leveling Apportionment of Outflow, and shown schematically in FIG. 35B.

The expandable structure is injection blow molded to include branches if applicable into the circumferential alternating pattern of flats and vaults described above in this section as well as in section 2a, entitled Background of the Invention. General, as well as in section 6, entitled Description of the Preferred Embodiments of the Invention. In a switch heterotopic double heart transplant wherein the second heart is positioned in the abdominal cavity, a shorter line is not used, and that longer which is implanted is made in the flats and vaults structure that will expand with growth and flex at the waist, eliminating the need for an alternative means for providing flexibility at the waist.

The expansion structure can be formed to replace any larger vessel and expand to accommodate growth. Made in two sizes, the larger is for the replacement of major vessels and made in different configurations to accommodate flaring and integral branches for use in the chest and abdomen. Flaring is not so pronounced that a pair of inline coupling jackets cannot be selected so that the thick foam lining will not accommodate both the narrower and broader ends of the native segment. That smaller—too small for replacing the thoracic arch in an infant, for example, is intended to replace vessels smaller in size to include most arteries. The latter are also suitable for use as druglines of the disorder response system. The thickness of the foam lining inline coupling and side-entry jackets and valves also supports increased growth.

Because upon expanding the circumferential vaults present entries inside the lumen, the optimization of laminar or streamline flow and the avoidance of an accumulation of clot analogous to that which collects in a saccular aneurysm acting as a trap upon initial placement is dispelled by dispensing the expandable conduit with the vaults 234 directed inward, or adaxially as close to coplanar with the flats as possible. Then as expansion with growth ensues, if not planar but rather directed inward, the vaults gradually become planar with the flats 234 before reversing protrusion outwardly, or abaxially. Moreover, the vaults are made relatively wide and shallow with gradually rounded entries and exits. Given the greater pliancy required of the vaults, manufacture is expedited by making the expandable material in two layers, an outer relatively stiff and an inner more pliant for the vaults.

Once the vaults become abaxial, other measures to avoid the accretion of clot include avoiding the use of thrombophilic materials, instead incorporating or coating the lumen walls with a thrombophobic, and if necessary, an anticoagulant released into the neoarch as an automatically released slow drip through a drugline 8 administered by the implanted disorder response system. In a neonate, the volume of a heparin, for example, is kept to the minimum to avoid bleeding and other complications. Using the expandable structure in a tiny patient, analogous means are used to dispel the obstruction of druglines and confluence conduits that would likewise become subject to obstruction due to coagulation. The structure described inherently provides proportional expansion in both the internal diameter, or caliber, and the length of the conduit.

The expansion structure also imparts pliancy which allows the prosthesis to be:
1. Bent as needed so that a prosthetic aortic arch, or neoarch, for example, addressed above in sections 2b(2)(c)(c1), entitled Inline Coupling Jackets Proper, and 2b(2)(c)(c2), entitled Automatic Insertion into a Ductus of an Inline Coupling Jacket-secured Bridging, or Tie-line and Its Replacement if Necessary, for example, conform to the curvature of the thoracic aorta.
2. Comply with propagation of the pulse without significant change in compliance during expansion.
3. Yield to, rather than protrude into or abrade against, adjacent tissue without significant loss in internal cross-section or increase in the pressure of the blood flowing through the prosthesis.
4. Allow some flattening of the prosthesis in a curve without significantly affecting the volume and pressure of flow through the prosthesis or persistence of the transition from streamline, or laminar, to turbulent flow past the prosthesis outlet.
5. Fit vessels over a significant range of sizes; and critically,
6. Afford a range of expansion in size such that provided placement in a child had been performed correctly, the need to reenter later due to growth should seldom if ever arise.

Thus, especially in an embodiment for a frail neonate or infant, addressed above in section 2b(2)(c)(c2), cited just above a prosthetic replacement aortic arch, or aortic neoarch, this pliancy not only facilitates conformity to the compound curve of the thoracic arch, the one convex craniad and sideways to the right and the other backwards to continue downwards as the thoracoabdominal, then abdominal aorta, but critically, allows at least a long initial term for the baby to gain in strength before having to undergo a follow-up operation to replace the device.

Where the material of the casing is coated to avert hydrolytic breakdown, it is likely that the need for revision will never arise; with sufficient expandability provided that all but a very large patient will reach adulthood without the need for revision or with revision many years after the initial operation. Information pertaining to tie-line type prostheses is presented above in section 2b(2)(c), entitled Vascular Segment Prostheses: Inline Coupling Jackets and Bridging, or Tie-Lines, and the sections subsidiary thereto.

2b(2)(g). Ischemia and Hypoperfusion-Free Carotid Endarterectomy Bypass and Prosthesis

Described here is the carotid bifurcation bypass device shown in FIGS. 25A and 25B, incorporating adjustable diversion servovalves of the type shown in FIGS. 10A thru 10C, which uni- or bilaterally as the patient requires, can be used:
a. During endarterectomy to eliminate the risk of ischemia or stroke, flow diversion total during the procedure, and the bypass thereafter removed. Diversion valve adjustability not required, bistable solenoid-controlled valves are used for full diversion during the procedure.
b. As a prosthesis to replace the native bifurcation, allowing excision thereof, eliminating the need for an endarterectomy or transluminal treatment of plaque, an aneurysm, or tumor for example. The prosthesis is then remotely controllable for drug delivery automatically. Here again, valve adjustability is not required, thus allowing the use of less costly bistable solenoid-controlled valves.
c. As both of the above—the bypass is used during endarterectomy and then left in place not only to allow drug delivery automatically but also to allow flow to be divided, that is, apportioned, between the bypass and the native bifurcation to dispel hyperperfusion and hypertension on the basis of pressure data fed to the implanted microcontroller from implanted sensors. Continuous dynamic adjustability thus requires the use of servovalves.
d. To monitor, diagnose, and promote healing of the endarterectomized carotids by automatically switching between the prosthesis and the endarterectomized carotids now anastomosed, delineated below are four levels of use.
e. With the bypass lines withdrawn postendarterectomy but the valves left in place to allow the direct release of drugs through the accessory channel or channels of that on the common carotid to medicate the bifurcation and those on the internal and external carotids to send medication to the ipsilateral brain or face respectively. Since the effective doses of drugs is based upon dilution and frequency of release, the valves can be either solenoid or servomotor driven, the latter also allowing adjustment in the relative proportion of flow through the internal or external carotid.
f. With the bypass lines withdrawn postendarterectomy but the servovalve on the common carotid left in place to allow dynamic adjustment in the volume of flow past it, the balance diverted through a basic side-entry jacket on the common carotid and through a line connected to a nonjacketing side-entry connector on a larger vessel such as the descending aorta or superior vena cava, wherein the added volume is proportionally inconsequential, the action driven by data from implanted pressure sensors fed to the implant controller.
g. With the bypass lines withdrawn postendarterectomy but the servovalve on the internal carotid left in place to allow dynamic adjustment in the volume of flow past it, the balance diverted through a basic side-entry jacket on the common carotid and through a line connected to a nonjacketing side-entry connector on a larger vessel such as the descending aorta or superior vena cava, wherein the added volume is proportionally inconsequential, the action driven by data from implanted pressure sensors fed to the implant controller.

The benefit of leaving the prosthesis in place includes more than automatic emergency responsive switching to it were the native structure to become impaired. If the carotids have remained intact, spontaneous and quickly responsive baro- and chemoreceptive function will remain functional when passage is through the native structure. Following endarterectomy, flow can be divided between the native and prosthetic lines to reduce shear stress and facilitate healing. By the same token, when passage is through the prosthesis, which incorporates blood pressure sensors and oximeters furnishing feedback to the implanted automatic disorder control system microcontroller or microprocessor, normal function is simulated.

FIG. 25A shows an ischemia and hypoperfusion-free carotid endarterectomy bypass implementation device devised to allow a common, internal, and external carotid artery operation without interruption in the flow of blood whether the substrate vessel will be repaired or replaced, while FIG. 25B the same type device for a patient whose external carotid is uninvolved. The bypass of circulation means that any debris generated during repair cannot continue to flow into the brain. This should eliminate the incidence of cardiovascular accidents such as intra- or postoperative stroke, or a cerebral or even a myocardial infarction. While consisting of separate valves, the device is meant to be sold as a unit apparatus with the small cables and other lines necessary.

That is, depending upon the state of degenerative disease or surgical or accidental trauma, the device can be used as an alternative, or bypass, pathway switched to during the procedure and/or during postprocedural healing, or if necessary, as a permanent prosthesis. Mid- and postoperatively, druglines 8 allow the targeting of medication directly into any or all of the three segments, which in itself can justify allowing the device to remain in place. These devices allow the endarterectomy to be performed not only without an interruption in blood flow, hence, without ischemia, but also without fluctuations in blood pressure.

The tight quarters for placement necessitate that the size and weight of the components be kept to a minimum with sharp corners and edges blunted, and if necessary, cushioned. Cushioning is with a parylene encapsulated viscoelastic polyurethane foam. FIGS. 25A and 25B show the valves somewhat enlarged for visual clarity. Also, the rotational angle of each valve is adjusted to least encroach upon neighboring tissue. The moat familiar cause for carotid obstruction and the need for endarterectomy is a buildup of atherosclerotic plaque on the interior surface of the lumen wall. This is not, however, the only cause of ischemic stroke, which includes cerebral aneurysms, hemorrhage, and arterial malformities to include carotid artery webs.

In some cases, the stenting of webs may be considered (see, for example, Mathew, S., Davidson, D. D., Tejada, J., Martinez, M., and Kovoor, J. 2021. “Safety and Feasibility of Carotid Revascularization in Patients with Cerebral Embolic Strokes Associated with Carotid Webs and Histopathology Revisited,” Interventional Neuroradiology 27(2): 235-240; Haynes, J. Raz, E., Tanweer, O., Shapiro, M. Esparza, R. and 11 others 2020. “Endarterectomy for Symptomatic Internal Carotid Artery Web,” Journal of Neurosurgery 28:1-8; Wojcik, K., Milburn, J., Vidal, G., Tarsia, J., and Steven, A. 2019. “Survey of Management Practices for Carotid Webs,” Ochsner Journal 19(4): 296-302; Coutinho, J. M., Derkatch, S., Polvin, A. R. J., Tomlinson, G., Casaubon, L. K., Silver, F. L, and Mandell, D. M. 2017. “Carotid Artery Web and Ischemic Stroke,” Neurology 88(1): 65-69 with correction 89(5): 521; Elmokadem, A. H., Ansari, S. A., Sangha, R., Prabhakaran, S. Shaibani, A., and Hurely, M. C. 2016. “Neurointerventional Management of Carotid Webs Associated with Recurrent and Acute Cerebral Ischemic Syndromes,” Interventional Neuroradiology 22(4): 432-437; Touze, E. Oppenheim, C. Trystram, D. Nokam, G. Pasquini, M., and 5 others 2010. “Fibromuscular Dysplasia of Cervical and Intracranial Arteries,” International Journal of Stroke 5(4): 296-305).

In the absence of carotid obstruction, smaller and less costly, basic side-entry jackets would serve well as end-connectors for the pipeline targeted delivery into the carotids of medication. Following an endarterectomy where valves such as those shown in FIGS. 25A and 25B had already been placed, rather than to remove and replace the valves with basic side-entry jackets, consideration should be given to the postprocedural advantages of switchability and shareability between native and prosthetic routes available to promote healing and assist in responding to a later emergency, such as a dissection or aneurysm, more likely in a patient with a connective tissue disorder.

The advantage of such follow-up support and the utility of the accessory channels in allowing the direct release into the internal carotids is likely to prove of value in elderly patients with both carotid obstruction and psychiatric problems that would have both the backup protection of the alternative routing and the direct delivery of medication through the valve accessory channels. Periodic switching from the native carotids to the prosthesis allows immediate diagnosis as to the state of healing and recovery of the native structure. Such intermittent switching between native and prosthetic carotids can be automatic in response to sensor inputs or manual.

Testing typically includes checking the response time to changes in blood pressure and glucose uptake by the brain responsive to shifts in posture, exertion, or intense emotion, for example. Upon alerting by these sensors, the controller commands the release of medication to restore function to within the normal range. The controller does this by signaling the outlet pump or drip stopcock of a small flat drug reservoir subcutaneously implanted in the pectoral region to release the medication in the dose specified in its prescription-program.

The drug or drugs released then pass through catheteric lines to be directly pipeline targeted through the servovalve accessory channel or channels to the common carotid, and if necessary, the valves on either or both its branches. Remedial measures might include reapportioning the volume of flow between the external and internal carotids, which too can be written into the prescription-program, which capability is lost if the mainlines, or bloodlines are removed to leave the accessory channels in place. Continued diversion or periodic diversion through the prosthesis postendarterectomy makes it possible to prevent shear stress while the intima and anastomoses heal. Reciprocally, periodic wetting of the intimae with blood can be beneficial.

There are, then, four levels of use: 1. As a bypass during carotid endarterectomy, 2. As a prosthesis left in place during post endarterectomy healing to allow switching between the native arteries and the prosthetic bypass for diagnostic and therapeutic purposes and to automatically activate in the event of an emergency, 3. As a permanent prosthesis left in place to allow switching between the two for diagnostic and therapeutic purposes and to automatically activate in the event of an emergency, and 4. As a permanent prosthesis where the native carotids had to be extirpated during tumor removal, aneurysm repair, irreversible disease, or accidental trauma. Positioning the Carotid endarterectomy is the most frequently performed surgical procedure with the loss or need for removal of the carotid baro- and chemoreceptors exceptional. Up to 1.6 percent of elderly patients with comorbidities undergoing a carotid endarterectomy experience a myocardial infarction, a preoperative evaluation of significant coronary artery occlusion most predictive (Clouse, W. D. and Brewster, D. C. 2004. “Cardiopulmonary Complications Related to Vascular Surgery,” in Towne, J. B. and Hollier, L. H. (eds.), Complication in Vascular Surgery, Second Edition, New York, New York: Marcel Dekker, pages 15-48).

Accordingly, should duplex ultrasound and/or magnetic resonance imaging or computer tomography angiography reveal a carotid having undergone an accumulation of plaque as to have become remodeled and severely dilated, tenuously stretched, malacotic, or otherwise impaired due to a vasculitis or some other vascular or connective tissue disease as would pose a risk of aneurysm, for example, a decision is made as to whether to retain and repair the native structure or to leave the bypass device with direct drug delivery accessory channels in place as a permanent prosthesis and excise the diseased tissue.

That the device shown in FIGS. 25A and 25B eliminates the deprivation of oxygen or the passage of microemboli to the brain should reduce if not eliminate the incidence of stroke, myocardial infarction, and postprocedural deficits in cognitive function, especially since the procedure is performed endoscopically under local or regional rather than general anesthesia, usually with the patient breathing spontaneously inside the oxygenated chest dome, with or without mechanical assistance. Using the side-entry vascular servovalve-based device shown in FIGS. 25A and 25B any debris released midprocedurally is trapped in the native carotid bulb and therefore bypassed.

A carotid bypass device such as that shown in FIGS. 10D and 25 makes possible an ischemia-free carotid endarterectomy under local anesthesia with spontaneous ventilation and freedom from the complications often encountered in a fraction of the time conventionally required. Where the native vessels are heavily diseased or have been irreparably damaged as the result of trauma or tumor resection, the device can be applied as a permanent prosthesis, the native tissue extirpated.

Usually, the device can be used midprocedurally with the postendarterectomized carotids anastomosed, in which case the vascular servovalves can be left in place with bloodlines, or mainlines, removed to allow continued use of the accessory channels by the automatic disorder response system. Alternatively, valves preserved for their accessory channels are replaced with upstream basic ductus side-entry jacket such as those shown in U.S. Pat. No. 11,759,186, entitled Ductus Side-entry Jackets and Prosthetic Disorder Response Systems. Measures to allow the valves to be minimized in size and weight and the considerations that allow this reduction addressed below.

The precise control over the servovalves essential to avert a temporary ischemic attack if not a stoke or myocardial infarction makes necessary the addition to a conventional side-entry vascular servovalve such as used to perform a metered compound vascular bypass, or sudden switched-organ transplantation of a mechanical fitting which allows the operator to directly observe and finely adjust the position of the diversion chute manually during the initial period of no current flow when the valves are applied. Such a side-entry vascular servovalve with direct manual assist is shown in FIGS. 10A thru 10C.

The fine control gained with a servovalve incorporating this feature may make such valves preferable for use in a metered switch transplant where a high degree of precision is desired. In a case where the results of imaging indicate that the best conventional technique would not achieve a cure, a carotid endarterectomy is never performed-instead, the prosthesis is applied as a permanent prosthesis ab initio. Due to the likelihood for troublesome swings in blood pressure upon the loss of carotid baro- and chemoreceptor function, provided one of the sides is more severely affected, an attempt is made to limit extirpation to the one side.

Experience with the consequences on blood pressure, blood gas homeostasis, and glucose regulation of a unilateral or bilateral loss of the carotid body, or glomus caroticum, is known from patients who had undergone a carotid body paraganglioma, glomus tumor, or malignant metastasis, which infiltrative, compelled resection. Specifically, baroreceptors are mechanosensory proteins located in the outer layer of the carotid sinus and the inner curvature of the aortic arch (Reutersberg, B., Pelisek, J., Ouda, A., de Rougemont, O., Rössler, F., and Zimmermann, A. 2022. “Baroreceptors in the Aortic Arch and Their Potential Role in Aortic Dissection and Aneurysms,” Online, MDPI [Multidisciplinary Digital Publishing Institute] Journal of Clinical Medicine (Basel, Switzerland) 11(5): 1161).

Resection of radiation necessitating resection of a segment along a major vessel such as the aorta will usually have been used to treat a cancer which would best be replaced with an inline coupling jacket-secured prosthesis as described above in section 2b(2)(c), entitled Vascular Segment Prostheses: Inline Coupling Jackets and Bridging, or Tie-lines and its subsidiary sections. Following a traumatic or surgical loss of baroreception and chemoreception where the native pressoreceptors could not be salvaged, the incorporation of sensors into an inline coupling jacket-secured prosthesis to simulate their contribution to normal baroreception in the stabilization of blood pressure and the heart rate by indicating changes in the total peripheral resistance and the cardiac output and chemoreception by indicating the blood saturation to control the rate and depth of respiration, will be important.

This especially when the patient is unable to compensate for the loss spontaneously with the aid of vision and proprioception (see, for example, Armstrong, M., Kemdt, C. C., and Moore, R. A. 2023. “Physiology, Baroreceptors,” Online, Treasure Island, Florida: StatPearls Publishing Co.; Brinkman, J. E., Toro, F., and Sharma, S. 2023. “Physiology, Respiratory Drive,” Online, Treasure Island, Florida: StatPearls Publishing Co.; Brognara, F., Castania, J. A., Kanashiro, A., Dias, D. P. M., and Salgado, H. C. 2021. “Physiological Sympathetic Activation Reduces Systemic Inflammation: Role of Baroreflex and Chemoreflex,” Online, Frontiers in Immunology (Lausanne, Switzerland) 12:637845; Castania, J. A., Katayama, P. L., Brognara, F., Moraes, D. J. A., Sabino, J. P. J., and Salgado, H. C. 2019. “Selective Denervation of the Aortic and Carotid Baroreceptors in Rats,” Online, Experimental Physiology (Cambridge, England) 104(9): 1335-1342; Van Vliet, B. N., Chafe, L. L., and Montani, J. P. 1999. “Contribution of Baroreceptors and Chemoreceptors to Ventricular Hypertrophy Produced by Sino-aortic Denervation in Rats,” Online, Journal of Physiology (Oxford, England) 516 (Part 3)(Part 3): 885-895).

Adaptation may prove insufficient, and radiation and/or surgery can directly damage baroreception and chemoreception. Each cause is rare but their sum prevalence is not (see, for example, Clarke, A. J, Swart, G., Clifford, A. R., Milross, C., Halmagyi, G. M., and Spies, J. 2023. “Baroreflex Failure as a Long-term Sequela of Head and Neck Irradiation,” Journal of Neurology (New York, New York) 270(5): 2784-2788; Lamotte, G., Coon, E. A., Suarez, M. D., Sandroni, P., Benarroch, E. E., and 8 others 2021. “Natural History of Afferent Baroreflex Failure in Adults,” Online, Neurology (Hagerstown, Maryland) 97(2): e136-e144; Kaufmann, H., Norcliffe-Kaufmann, L., and Palma, J. A. 2020. “Baroreflex Dysfunction,” New England Journal of Medicine (Boston, Massachusetts) 382(2): 163-178; Piani, F., Landolfo, M., Fiorini, G., D'Addato, S., Mancia, G., and Borghi, C. 2020. “Severe Impaired Blood Pressure Control Caused by Baroreflex Failure as a Late Sequela of Neck Irradiation,” Journal of Hypertension (Alphen aan den Rijn, Netherlands) 38(3): 553-556; Shimada, M., Singh, A., and Ejaz, A A. 2020. “Baroreflex Dysfunction,” New England Journal of Medicine (Boston, Massachusetts) 382(16): 1577-1578; Vacirca, A., Faggioli, G., Pini, R., Freyrie, A., Indelicato, G., and 6 others 2020. “Predictors of Survival in Malignant Aortic Tumors,” Online, Journal of Vascular Surgery ((New York, New York) 71(5): 1771-1780; Toner, A., Jenkins, N., and Ackland, G. L,” 2016. “Baroreflex Impairment and Morbidity after Major Surgery,” Online, British Journal of Anaesthesia (London, England) 117(3): 324-31).

Radiation, especially if excessive as might occur in the attempt to eradicate head or neck cancer, often results in deficits of baroreception which can lead to a cardiac arrest, recommending the administration of dysrhythmia counteractive medication and/or the placement of an implantable pacemaker/cardioverter-defibrillator when radiation used to treat a head or neck tumor has been confirmed to have resulted in a radiation-induced afferent baroreflex failure (Biaggioni, I., Shibao, C. A., and Jordan, J. 2022. “Evaluation and Diagnosis of Afferent Baroreflex Failure,” Hypertension 79(1): 57-59; Lamotte, G H., Coon, E. A., Suarez, M. D., Sandroni, P., Benarroch, E. E., and 7 others 2022. “Standardized Autonomic Testing in Patients with Probable Radiation-induced Afferent Baroreflex Failure,” Hypertension 79(1): 50-56; Lamotte, G H., Coon, E. A., Suarez, M. D., Sandroni, P., Benarroch, E. E., and 8 others 2021. “Natural History of Afferent Baroreflex Failure in Adults,” Neurology 97(2): e136-e144; Yaxley, J. and Scott, T. 2019. “Recurrent Pulseless Electrical Activity and Cardiac Arrest Caused by Baroreceptor Failure following Neck Irradiation,” Turkish Journal of Emergency Medicine 19(4): 152-153; Xu, W. X., Yu, J. L., Feng, Y., Yan, Q. X., Li, X. Y., and 8 others 2018. “Spontaneous Activities in Baroreflex Afferent Pathway Contribute Dominant Role in Parasympathetic Neurocontrol of Blood Pressure Regulation,” Online, CNS Neuroscience and Therapeutics (Oxford, England) 24(12): 1219-1230; Janus, S. E., Hirai, T., Mathew. V., and Rabbat, M. 2017. “Syncope from Radiation-induced Baroreceptor Failure,” Online, British Medical Journal Case Reports 2017: ber2017221925; Sharabi, U., Dendi. R., Holmes. C. and Goldstein, D. 2003. “Baroreflex Failure as a Late Sequela of Neck Irradiation,” Hypertension 42(1): 110-116; Cheng, S. W., Wu. L. L., Ting. A. C., Lau. H/, Lam. L/K/, and Wei. W. I. 1999. “Irradiation-induced Extracranial Carotid Stenosis in Patients with Head and Neck Malignancies,” American Journal of Surgery 178(4): 323-328; Timmers, H. J., Karemaker, J. M., Lenders, J. W., and Wieling, W. 1999. “Baroreflex Failure following Radiation Therapy for Nasopharyngeal Carcinoma,” Clinical Autonomic Research 9(6): 317-324; Zidar, N., Ferluga, D., Hvala, A., Popović. M., and Soba, E. 1997. “Contribution to the Pathogenesis of Radiation-induced Injury to Large Arteries,” Journal of Laryngology and Otology 111(10): 988-990; Cheng. V. S. and Schultz, M. D 1975. “Unilateral Hypoglossal Nerve Atrophy as a Late Complication of Radiation Therapy of Head and Neck Carcinoma: A Report of Four Cases and a Review of the Literature on Peripheral and Cranial Nerve Damages after Radiation Therapy,” Cancer 35(6): 1537-1544).

While the need for removal, especially bilateral, is rare, the consequences of a loss in carotid baroreception, chemoreception, coordinated function of the solitary nucleus (nucleus tractus solitarii) in the medulla oblongata, a segment of the carotid sinus nerve, and the impairment in cerebral autoregulatory function of the blood pressure secondary to the disabling or extirpation of the carotid bulb or bulbs, much less further related physiology cannot be more than incompletely addressed below.

The consequences of even a bilateral resection tend to subside over time and are medically manageable even without the immediate support of an implanted automatic response system that instantly detects and is able to respond to excessive deviations in blood pressure, heartrate (tachycardia), imbalances in blood gases, and/or glucose levels with medication and electrostimulation, or neuromodulation, as appropriate. This instancy of diagnosis and therapeutic response, which could not have been anticipated in the existing literature, is critically different than the current situation, where medical assistance usually cannot be obtained for many hours if not days.

When a bilateral resection is unavoidable, removal is staged so that only the side more severely affected is operated on for as long a time as possible. Malignant tumors force resection of the contralateral tumor more promptly than do tumors that infiltrate the adjacent neurology as would prove too traumatizing to allow separation and removal (see, for example, Demir. T., Uyar, I., Bolgi Demir, H., Sahin, M., and Gundogdu, G. 2014. “Five-year Follow-up of a Patient with Bilateral Carotid Body Tumors after Unilateral Surgical Resection,” American Journal of Case Reports 15:426-430). Persistent fluctuations in blood pressure, typically hypertensive, and glucose metabolism appear not to result (see, for example, Timmers, H. J. L. M., Wieling, W., Karemaker, J. M., and Lenders, J. W. M. 2003. “Denervation of Carotid Baro- and Chemoreceptors in Humans,” Journal of Physiology 553 (Part 1): 3-11).

The need for a follow-up patch angioplasty procedure and potential complications as could significantly extend the duration of the procedure or necessitate reentry or a transluminal intervention, primarily stroke, and the possibility of injury to one or more cranial nerves—the hypoglossal, vagus, recurrent laryngeal, marginal mandibular, superior laryngeal, glossopharyngeal, spinal accessory, transverse cervical, and greater auricular (Rockman, C. and Riles. T. S. 2004. “Nonstroke Complications of Carotid Endarterectomy,” in Towne. J. B. and Hollier, L. H. (eds), Complication in Vascular Surgery, Second Edition, New York, New York: Marcel Dekker, pages 475-482)—should be evaluated, as should the risk for the rupture of a vein patch if created.

Unlike a strictly organic surgical treatment, the prosthesis includes means for detecting and instantly responding to a need for medication to be directly targeted to the treatment site. Although a need for bilateral removal of the carotid bulbs is rare and replacement with prostheses best avoided, if bilateral resection, hence, denervation, and therewith, the disabling of carotid baroreceptive function cannot be avoided, the prostheses are equipped with pressure and chemical sensors to signal the implanted controller to instantly release quick onset of action blood pressure adjusting medication through the servovalve accessory channels as appropriate. Drugs for directly pipeline targeted application bypass the liver and must be converted into the post-liver metabolized form.

For directly pipeline targeted application, antihypertensives such as calcium channel blockers, angiotensin converting enzyme inhibitors, angiotensin receptor blockers, thiazide-diuretics, and thiazide-diuretics should not require post hepatic and postrenal first pass conversion. Calcium channel blockers, for example, directly affect the nephrons, and exposure of the liver to these drugs can produce adverse hepatic and secondary side effects. Direct pipeline-targeting eschews the toxic side effects of which such drugs are capable and by bypassing nontargeted tissue, allows targeted drugs to be administered at dosage levels that could not be dispersed throughout the systemic circulation.

The reinstatement of automated means to replace innate baro- and chemoreception when exceptionally the innate receptors must be removed with the defective tissue is addressed just below in this section and inter alia above in section 2b(2)(c), entitled Vascular Segment Prostheses: Inline Coupling Jackets and Bridging, or Tie-lines. Responding within seconds regardless of patient location or mental state is critically superior to current therapy.

Essentially, a bilateral removal of the innate sensors results primarily in fluctuations in blood pressure unresponded to within the normally brief interval following the operation, and extremely rare, excision of carotid body tumors results in some deficit in chemoreception (see, for example, Armstrong, M., Kerndt. C. C., and Ross A. M. 2022. “Physiology, Baroreceptors,” Online, Treasure Island, Florida: StatPearls Publishing Co.; Gold, O. M. S., Bardsley, E. N., Ponnampalam, A. P., Pauza, A. G., and Paton, J. F. R. 2022.” Online, Frontiers in Synaptic Neuroscience 14:902319; MacMillan, S., Holmes, A. P., Dallas, M. L., Mahmoud, A. D., Shipston, M. J. and 4 others 2022. “LKB1 [liver kinase B1] is the Gatekeeper of Carotid Body Chemosensing and the Hypoxic Ventilatory Response,” Online, Communications Biology 5(1): 642 erratum 5(1): 765; Brognara, F., Felippe. I. S. A., Salgado, H. C., and Paton, J. F. R. 2021. “Autonomic Innervation of the Carotid Body as a Determinant of Its Sensitivity for Cardiovascular Physiology and Pathology,” Online, Cardiovascular Research 117(4): 1015-1032; Iturriaga. R., Alcayaga, J. Chapleau, M. W., and Somers, V. K. 2021. “Carotid Body Chemoreceptors: Physiology, Pathology, and Implications for Health and Disease,” Online, Physiological Reviews 101(3): 1177-1235; Pirahanchi, Y., and Bordon, B. 2021. “Anatomy, Head and Neck, Carotid Baroreceptors,” Online, Treasure Island, Florida: StatPearls Publishing Co.; Kotsis. T., Christoforou P., and Nastos. K. 2020. “Carotid Body Baroreceptor Preservation and Control of Arterial Pressure in Eversion Carotid Endarterectomy,” Online, International Journal of Angiology 29(1): 33-38; Stocco, E., Barbon, S., Totorella, C., Macchi, V. DeCaro, R., and Porsionato, A. 2020. “Growth Factors in the Carotid Body—An Update,” Online, MDPI [Multidisciplinary Digital Publishing Institute] International Journal of Molecular Sciences (Basel, Switzerland) 21(19): 7267; Iturriaga, R. 2018. “Carotid Body Ablation: A New Target to Address Central Autonomic Dysfunction,” Current Hypertension Reports 20(6): 53; Porzionato, A., Macchi, V., Stecco, C., and De Caro, R. 2018. “The Carotid Sinus Nerve-Structure, Function, and Clinial Implications,” Online, Anatomical Record 302(4): 575-587; Celi de la Torre, J. A., Skrypnik, D. A., Vinogradov. R. A., Böckler, D., and Demirel, S. 2018. “Postoperative Alterations in Blood Pressure after Carotid Endarterectomy: Implications of Different Reconstruction Methods,” [in German with English abstract at Pubmed] Chirurgie 89(2): 123-130; Demirel. S., Goossen, K., Bruijnen, H. Probst, P., and Böckler, D. 2017. “Systematic Review and Meta-analysis of Postcarotid Endarterectomy Hypertension after Eversion versus Conventional Carotid Endarterectomy,” Online, Journal of Vascular Surgery (New York, New York) 65(3): 868-882; Ben Ahmed, S. Daniel, G., Benezit, M., Bailly, P., Aublet-Cuvelier, B., and 3 others 2015. “Does the Technique of Carotid Endarterectomy Determine Postoperative Hypertension?. Annals of Vascular Surgery 29(6): 1272-1280; Del Rio. R., Andrade, D. C., Marcus, J. J., and Schultz, H. D. 2015. “Selective Carotid Body Ablation in Experimental Heart Failure: A New Therapeutic Tool to Improve Cardiorespiratory Control,” Online, Experimental Physiology 100(2): 136-142; Niizuma, K., Shimizu, H., Inoue. T. Watanabe, M. and Tominaga, T. 2014. “Maximum Preservation of the Media in Carotid Endarterectomy,” Online, Neurologia Medico-chirurgica (Tokyo, Japan) 54(10): 812-818; Jagtap, S. R., Bakhshi, R. G., Khatavkar, S. S., Phadtare, S. J., and Mohite, S. N. 2013. “Carotid Body Tumor Excision: Anaesthetic Challenges a Review of Literature,” Online, Indian Journal of Anaesthesia 57(1): 76-78; Hanley, D. F., Wilson, D. A., Feldman, M. A., and Traystman, R. J. 1988. “Peripheral Chemoreceptor Control of Neurohypophysial Blood Flow,” American Journal of Physiology. Heart and Circulatory Physiology (Bethesda, Maryland) 254(4 Part 2): H742-H750; Traystman, R. J. and Fitzgerald, R. S. 1981. “Cerebrovascular Response to Hypoxia in Baroreceptor and Chemoreceptor-denervated Dogs,” American Journal of Physiology. Heart and Circulatory Physiology 241(5): H724-H731; Hickey, R. F., Ehrenfeld, W. K., Hamilton, F. N., and Larson, C. P. Jr. 1972. “Bilateral Carotid Endarterectomy with Attempted Preservation of Carotid Body Function,” Online, Annals of Surgery (Philadelphia, Pennsylvania) 175(2): 268-273; Wade, J. G., Larson, C. P Jr., Hickey, R. F., Ehrenfeld, W. K., and Severinghaus, J. W. 1970. “Effect of Carotid Endarterectomy on Carotid Chemoreceptor and Baroreceptor Function in Man,” New England Journal of Medicine 282(15): 823-829).

Comparable to the loss of baro- and chemoreceptive function originating within the aortic arch, where resection necessitated by aneurysmal repair, for example, is followed by gradual recovery, a reinstatement of response time or performance level of adaptation can be approximated using electronic means, but left to itself, will never fully recover to that level afforded by intact receptors or a capable automated system.

With the implanted automatic response system, the level of therapy subsides as does the havoc, thus materially improving the quality of life. Preferably, postoperative retention of the native carotid structures can be accomplished bilaterally with the accessory channels coursing through the servovalves used to directly target a statin, known to have topical efficacy, and an anti-inflammatory into the common carotid to pass through both the internal and external carotids.

Alternatively, the internal carotid is directly targeted. With either, a background dose of a statin and any other drug judged essential can be dispersed throughout the circulatory system by any conventional route as well as directly pipeline targeted to other loci or nidi. Most of the literature pertaining to carotid baro- and chemoreception concern pathological conditions affecting these when the anatomy remains intact rather than following resection. Normal function and the consequences of removal pertinent here, references concerned with the effect of various pathological conditions have been omitted.

The control of blood pressure is through a network of baroreceptive, hormonal, and renal components, each having wider control in that order. Deviations from the normal range in blood pressure following baroreceptive dysfunction or resection are still returned to within the normal range within minutes by the hormonal component, joined by the renal fluid pressure control system within hours, showing that baroreception originating within the carotid bulb exerts its control over the blood pressure more quickly but hardly exclusively (Guyton, A. C. 1991. “Blood Pressure Control-Special Role of the Kidneys and Body Fluids,” Science 252(5014): 1813-1816).

The resection of paraganglionic tumors may cause damage to the carotid body, and sometimes necessitates its removal (Ghali, M. G. Z., Srinivasan, V. M., Hanna, E., and DeMonte, F. 2017. “Overt and Subclinical Baroreflex Dysfunction after Bilateral Carotid Body Tumor Resection: Pathophysiology, Diagnosis, and Implications for Management,” World Neurosurgery 101:559-567)(see also Biaggioni, I., Shibao, C. A., Diedrich, A., Muldowney, J. A. S. 3rd, Laffer, C. L., and Jordan, J. 2019. “Blood Pressure Management in Afferent Baroreflex Failure: JACC Review Topic of the Week,” Journal of the American College of Cardiology 74(23): 2939-2947; Limberg, J. K, Taylor, J. L., Mozer, M. T., Dube, S., Basu, A., and 5 others 2015. “Effect of Bilateral Carotid Body Resection on Cardiac Baroreflex Control of Blood Pressure during Hypoglycemia,” Hypertension 65(6): 1365-1371; Demir, T., Uyar, I., Bolgi Demir, H., Sahin, M., and Gundogdu, G. 2014, Op cit. in this section; Robertson, D., Diedrich, A., and Chapleau, M. 2012. “Afferent Baroreflex Failure,” Autonomic Neuroscience: Basic and Clinical 172(1-2): 1-3; Timmers, H. J. L. M., Wieling, W., Karemaker, J. M., and Lenders, J. W. M. 2003, Op cit. in this section; Gur, I. and Katz, S. 2010. “Baroreceptor Failure Syndrome after Bilateral Carotid Body Tumor Surgery,” Annals of Vascular Surgery 24(8): 1138.e1-3; Kougias, P., Weakley, S. M., Yao, Q. Lin, P. H. and Chen, C. 2010. “Arterial Baroreceptors in the Management of Systemic Hypertension,” Medical Sciences Monitor 16(1): RAI-RA8; De Toma, G., Nicolanti, V., Plocco, M., Cavallaro, G., Letizia, C., Piccirillo, G., and Cavallaro, A. 2000. “Baroreflex Failure Syndrome after Bilateral Excision of Carotid Body Tumors: An Underestimated Problem,” Journal of Vascular Surgery (New York, New York) 31(4): 806-810; Guyton, A. C. 1991, Op cit. in this section).

FIGS. 25A and 25B show a ductus side-entry vascular servovalve-based bypass devices, or appliances, for performing a carotid endarterectomy without ischemia, hyperperfusion syndrome, extravasation, increased obstruction to blood flow, the risk of releasing debris or a thromboembolus that mid- or postprocedurally could cause what is effectively a temporary ischemic attack if not a stroke or even a myocardial infarction. To eliminate needless detail, the anatomy has been schematized in omitting several vascular ‘twigs’ that supply neighboring structures such as muscles and the thyroid gland, which if bypassed for a brief period should not result in persistent sequelae attributable to the temporary ischemia.

In fact, each common carotid and its branches is protected through contralateral collateral communication-” . . . by the free communication between the carotid arteries of opposite sides” (Gray, H. Anatomy of the Human Body, Goss, C. M. (ed.), 1966. Philadelphia, Pennsylvania: Lea and Febiger, page 582). Philadelphia, Pennsylvania: Lea and Febiger, page 582). However, where the placement of a bypass cuts off a contralateral branch completely, collateral circulation to branches of the contralateral external carotid may prove inadequate, and collateral circulation from the contralateral common and internal carotid to the ipsilateral carotid cannot provide sufficient oxygen to the brain to prevent a number of serious consequences. Over the time needed to perform a carotid endarterectomy, cutting off the small branches of the external carotid should leave the patient without any lasting adverse effects.

A temporary loss of sensation in the tongue, for example, should quickly dissipate. Where, however, carotid disease, a vasculitis, connective tissue disorder, tumor resection, or trauma makes retention of the bypass device as a permanent prosthesis imperative, alternative measures must be instituted to make certain that unless spontaneously accommodated by collateral circulation, bypassed branches of the external carotid are not completely cut off. The best solution is to create anastomoses between these and neighboring arteries not of carotid origin. Of the branches of the external carotid artery included in FIGS. 25A and 25B, ST is the superior thyroid artery, L is the lingual artery, F the facial artery, M the maxillary artery, and SupT the superior temporal artery.

Jacketing the carotids poses a problem of encroachment of the diversion valves upon neighboring tissues. The common carotid reaches downward to just short of the root of the neck, where the anatomy is compact toward the central axis of the body but the integument is loose enough that a valve with side stem directed away from the exterior can be placed to neither irritate adjacent tissue nor pose a cosmetic problem. The same cannot be said for the external and internal carotids which arise from the common carotid at about the jawline, “opposite the superior border of the thyroid cartilage” (Goss, C. M. (ed.) 1966, Op cit. in this section).

As in many other loci where the anatomy is compact, it is important to emphasize that a local constriction as allows the diversion jacket or valve to be significantly reduced in size does not in itself provoke medical problems. Mere constriction is readily adapted to; it is the release of microemboli from atherosclerotic plaques in conjunction with arterial narrowing that result in cerebral and coronary infarction. Once the narrowing and diseased tissue have been eliminated, here, through a carotid endarterectomy, the patient should not experience an ischemic event, or accident, any more than before the stroke or heart attack when the artery had adapted while no release of debris had occurred.

Where practicable, the lumen of mainlines, or bloodlines, consisting of the trepan tube in the diversion jacket or valve side stem and the tubing to which the trepan tube is connected, is made as close in caliber to that of the substrate vessel as possible. However, where the perivascular tissue (less functional perivascular fat which if thick can be trimmed) can be jacketed along with the vessel itself) is tightly fitted about the vessel, once the diseased condition is eliminated, can be jacketed with a much smaller jacket or valve, the body equipped with means for adjusting to a reasonable reduction in the flow-through cross-sectional area for the blood to pass.

Following plaque removal, narrowing the carotids to allow a significant reduction in the size and weight of the diversion servovalves eliminates the risk of thromboembolism release from within the common or internal carotid. However, the patient having presented the condition will have atherosclerotic disease, leaving the constricted segment more vulnerable to the passage through the segment or its occlusion by a thromboembolism of remote origin. Hence, if the bypass device is used only during the endarterectomy so that the native tissue remains, the carotids may become restenosed, making the regular release of a statin imperative and ezetimibe appropriate.

In a patient with familial hypercholesterolemia, the development of significant carotid blockages indicates that the proprotein convertase subtilisin/kexin-like 9 (PCSK9) RNA interference inhibitor such as evolucumab with ezetimibe had not been used and should now be considered imperative. If the carotids had been so diseased due to pathology other than dyslipidemic or have been left irreparable due to tumor removal or trauma that the bypass must be left in place as a permanent prosthesis, then the narrowing of the carotids by the prosthesis should not require treatment.

As exemplary for similar situations elsewhere in the body, these considerations mean that the diversion servovalves on the internal and external carotids used to create a bypass in order to allow an ischemia-free carotid endarterectomy can be made small enough as not to create postoperative problems that cannot be corrected by endoscopically rotating these and/or placing a cushioning filler. It warrants emphasis that the extracranial direct pipeline targeting of drugs into the supply territory of either internal carotid is not tied to a carotid bypass and prosthesis.

Direct pipeline targeting to the supply territory of the basilar artery, that is, the posterior or caudal brain, brainstem, upper spinal cord, and cerebellum, is into the basilar artery, access thereto and placement of a small side-entry jacket an intricate microsurgical procedure (see, for example, Piccinin, M. A. and Munakomi, S. 2019. “Neuroanatomy, Vertebrobasilar System,” Treasure Island, Florida: StatPearls Publishing, Online at ncbi.nlm.nih.gov/books/NBK540995/; Reinemeyer, N. E., Tadi, P., and Lui, F. 2019. “Basilar Artery Thrombosis,” Treasure Island, Florida: StatPearls Publishing, Online, at nebi.nlm.nih.gov/books/NBK532241/; Satapathy, B. C. and Mohapatra, C. 2018. “Morphometric Study of Basilar Artery in the Eastern Indian Population,” Asian Journal of Neurosurgery 13(3): 689-692; Sultana, N., Khalil, M., Khan, M. K., Kabir, A., Farjan, S., and 5 others 2018. “Variation in the Position and Diameter of Basilar Artery in Different Ages of Bangladeshi People,” Mymensingh Medical Journal (Mymensingh, Bangladesh) 27(3): 504-507; Mamatha, H., D'Souza, A. S., Pallavi, and Suhani, S. 2012. “Human Cadaveric Study of the Morphology of the Basilar Artery,” Singapore Medical Journal 53(11): 760-763; Saugstad, L. F. 2008. “What is a Psychosis and Where is it Located?,” European Archives of Psychiatry and Clinical Neuroscience 258 Supplement 2:111-117; Honeybul, S., Neil-Dwyer, G., Evans, B. T., and Lang, D. A. 1997. “The Transzygomatic Approach: An Anatomical Study,” British Journal of Oral and Maxillofacial Surgery 35(5): 334-340).

To avoid the side effects elsewhere in the body of drugs intended to treat the brain, connection of the same kind as shown in FIG. 16, U.S. Pat. No. 11,759,186, entitled Ductus Side-entry Jackets and Prosthetic Disorder Response Systems, where it is the heart that is directly targeted through the left anterior descending coronary, except that to directly target the brain, the line is connected to the internal carotids and/or the basilar artery. The drug or drugs are commanded to be released according to the schedule specified in a prescription-program written to allow adaptability for a range of similar patients.

The program is executed by an implanted microcontroller which actuates the outlet pump or drip stopcock of a small flat drug reservoir implanted subdermally in the pectoral region. Elderly patients will often require several such drug reservoirs and targeting lines, necessitating command by a microprocessor programmed to treat multiple comorbidities through a hierarchical or nested complex control system, as addressed in the foregoing patent. Drugs are replenished transcutaneously by injection into small flat subdermally positioned drug reservoirs through a small subdermally implanted port tube socket, tiny tattoo marks indicating the points for injection of the different drugs.

To minimize human errors, when multiple drugs are required, injection can be automated, in that a multiple injection needle base-plug similar to a vacuum tube socket plug base having the injection needles positioned according to a prescribed arrangement complementary to, the openings in the subdermal port thought of as the matching socket, allows replenishing the reservoirs simultaneously. When the operator inserts the plug through the skin of the patient and into the port and pushes a switch, each of the pumps in the bank thereof contained in a common console pumps the proper amount of the drug into each reservoir.

The microprocessor then administers each drug according to its respective schedule. That timely delivery of the prescribed dose proceeds automatically means that even if the patient, often elderly and requiring numerous drugs, is regimen noncompliant due to aversity, dementia, or psychosis, adherence to the regimen will be assured. An implanted automatic disorder response system is described and illustrated in FIGS. 12A and 12C, in U.S. Pat. No. 11,013,858, entitled Nonjacketing Side-entry Connectors and Prosthetic Disorder Response Systems.

As a prosthesis with retention of the endarterectomized carotid, when the diversion chute of the bypass inflow and outflow servovalves are retracted, drugs released through the inflow valve accessory channel or channels allow targeting medication such as a statin or steroid for uptake in the preserved common carotid. If not stopped from further travel into the internal and external carotids through the release of a reversal agent and/or by magnetic extraction when administered in the form of a superparamagnetic carrier-bound nanoparticulate drug, flow continues delivery of the drug to treat both the brain and face.

Drugs released through the accessory channel or channels of either bypass outflow servovalve continue on to either the brain or the face. Magnetic uptake requires the placement of a perivascular impasse jacket about the segment to be medicated. Magnetic perivascular jackets are described in U.S. Pat. No. 11,389,171, entitled Integrated System for the Infixion and Retrieval of Implants. This release of drugs is automatic according to the implant microcontroller prescription-program, and can be scheduled or responsive to data returned to the implant microcontroller or microprocessor by implanted sensors.

Excision of an aneurysm or tumor so that the native bifurcation is missing still allows the passage of drugs directly into either the internal or external carotid and so does nothing to detract from the ability of the prosthesis to selectively target drugs to the brain, the face, or both. Drugs to the brain should work topically, that is, without passage through and alteration by the liver, and can be supplemented with mannitol, for example, as one strategy to pass through the blood brain barrier. Alternatively, the preparation of drugs for direct targeting thus should include passage through a liver or the synthetic equivalent thereof. With the endarterectomized bifurcation intact, flow through the native bifurcation allows it to be medicated, while flow past the junction can directly target the brain or the face.

As indicated, apportioning flow between the bypass and the native bifurcation by partially retracting the servovalve diversion chutes allows adjustment in the blood pressure, which can proceed automatically on the basis of implanted pressure sensors. The importance of the ability to selectively target drugs to the brain or to the face cannot be overstated. Nonsteroidal anti-inflammatory analgesics can be targeted directly to the brain without the risk of gastrointestinal irritation or ulceration. Reciprocally, magnetic perivascular ductus side-entry jackets positioned at target segments along the gut allow the direct targeting of lesions to include Crohn's with superparamagnetic carrier-bound prednisone, methylprednisolone, or any other magnetically susceptible-carried drug in fluid form, and other type lesions with any other carried drug in fluid form.

Ductus Side-entry Jackets and Prosthetic Disorder Response Systems are described and illustrated in U.S. Pat. No. 11,759,186 of like title, which shows a magnetized ductus side-entry jacket in FIG. 4, the same with a radiation shield for use with low to moderate dose-rate radioisotopes in FIG. 5, and the same with a disintegrating shield for temporary use in FIG. 6. Targeting thus allows substantially eliminating side effects elsewhere in the body, such as ‘moon facies’ due to the use of steroids to target Crohn's lesions.

That any drug or radioisotope delivered in fluid form can be targeted directly to the brain through the internal carotids automatically on time has important neuropsychiatric implications, the more so given that administration is automatic and not dependent upon compliance or having to deal with a demented or psychotic agitated, averse, excitable, or unruly patient. While the unwanted effects of some neuroleptic, or antipsychotic, drugs affect the brain itself to cause tardive dyskinesia, tremor, drowsiness, oculogyric crisis, and blurred vision among others, the drugs can also affect other parts of the body.

For the former, which necessitates discrimination and targeting of functionally distinct tissues within the brain, oculogenetics should provide some answers. For the latter, direct piping to the target site holds forth the hope of relief. As do all drugs, those used to alleviate the side effects of antipsychotic drugs, such as valbenazine and deutetrabenazine can cause their own side effect-drowsiness, or somnolence, and valbenazine can cause QT segment prolongation, although the risk of triggering tachycardia is slight. Antipsychotic drugs are numerous and all carry the risk of adverse side effects which to counteract requires the use of other drugs which may cause side effects, but less serious or severe than those produced by the primary drug.

To allow infusion or injection, most if not all antipsychotics are already available in liquid form. Direct targeting that eliminates passage through the liver to obtain efficacy of potency were these applied directly and prodrugs must be formulated to duplicate the liver-processed end product (see, for example, Sajatovic, M., Alexopoulos, G. S., Burke, J., Farahmand, K., and Siegert, S. 2020. “The Effects of Valbenazine on Tardive Dyskinesia in Older and Younger Patients,” International Journal of Geriatric Psychiatry 35(1): 69-79; Arya, D., Khan, T., Margolius, A. J., and Fernandez, H. H. 2019. “Tardive Dyskinesia: Treatment Update,” Current Neurology and Neuroscience Reports 19(9): 69; Beauchemin, M., Geguchadze, R., Guntur, A. R., Nevola, K., Le, P. T., and 6 others 2019. “Exploring Mechanisms of Increased Cardiovascular Disease Risk with Antipsychotic Medications: Risperidone Alters the Cardiac Proteomic Signature in Mice,” Pharmacological Research 152:104589; Dumna, S. R. and Fung. V. S. 2019. “Drug-induced Movement Disorders,” Australian Prescriber 42(2): 56-61; Ferreira, T. F., Godinho, F. D. G., Dehanov, S. D., Trancas, B., and Maia, T. 2019. “Olanzapine-induced Fever,” Primary Care Companion for CNS Disorders (Memphis, Tennessee) 21(1). pii: 18102332; May, M., Beauchemin, M., Vary, C., Barlow, D., and Houseknecht, K. L. 2019. “The Antipsychotic Medication, Risperidone, Causes Global Immunosuppression in Healthy Mice,” Online, PLoS [Public Library of Science] One 14(6): e0218937; Musco, S., Ruekert, L., Myers, J., Anderson, D., Welling, M., and Cunningham, E. A. 2019. “Characteristics of Patients Experiencing Extrapyramidal Symptoms or Other Movement Disorders Related to Dopamine Receptor Blocking Agent Therapy,” Journal of Clinical Psychopharmacology 39(4): 336-343; Nasr Esfahani. P., Nasiri, J., Badihian, S., and Yaghini, O. 2019. “Short-term Side Effects of Low Dose Valproate Monotherapy in Epileptic Children: A Prospective Study,” Iranian Journal of Child Neurology 13(2): 37-46; Kakunje, A., Prabhu, A., Sindhu Priya, E. S., Karkal, R., Kumar, P., Gupta, N., and Rahyanath, P. K. 2018. “Valproate: It's Effects on Hair,” International Journal of Trichology 10(4): 150-153; Pieters, L. E., Bakker, P. R., and van Harten, P. N. 2018. “Asymmetric Drug-induced Parkinsonism and Psychopathology: A Prospective Naturalistic Study in Long-stay Psychiatric Patients,” Online, Frontiers in Psychiatry 9:18; Ricciardi, L., Pringsheim, T., Barnes, T. R. E., Martino, D., Gardner, D., and 6 others 2019. “Treatment Recommendations for Tardive Dyskinesia,” Canadian Journal of Psychiatry 64(6): 388-399; Vicens. C., Leiva. A., Bejarano, F., Sempere, E., Rodriguez-Rincón, R. M. and 10 others 2019. “Intervention to Reduce Benzodiazepine Prescriptions in Primary Care, Study Protocol of a Hybrid Type 1 Cluster Randomised Controlled Trial: The BENZORED [benzodiazepine reduction] Study,” Online, British Medical Journal Open 9(1): e022046; Zareifopoulos. N. and Panayiotakopoulos, G. 2019. “Treatment Options for Acute Agitation in Psychiatric Patients: Theoretical and Empirical Evidence,” Online, Cureus (Palo Alto, California) 11(11): e6152; Caroff, S. N., Aggarwal. S., and Yonan, C. 2018. “Treatment of Tardive Dyskinesia with Tetrabenazine or Valbenazine: A Systematic Review,” Journal of Comparative Effectiveness Research 7(2): 135-148; Carulla-Roig. M., Isomura, K., Perez-Vigil. A., Larsson, H., Hellner, C., Mataix-Cols, D., and Fernandez de la Cruz, L. 2018. “Pharmacoepidemiology of Tourette and Chronic Tic Disorders in Sweden 2005-2013,” Journal of Child and Adolescent Psychopharmacology 28(9): 637-645; Cummings, M. A., Proctor, G. J., and Stahl, S. M. 2018. “Deuterium Tetrabenazine for Tardive Dyskinesia,” Clinical Schizophrenia and Related Psychoses 11(4): 214-220; Niemann, N. and Jankovic, J. 2018. “Treatment of Tardive Dyskinesia: A General Overview with Focus on the Vesicular Monoamine Transporter 2 Inhibitors,” Drugs (Auckland, New Zealand) 78(5): 525-541; Sarva, H. and Henchcliffe, C. 2018. “Valbenazine as the First and Only Approved Treatment for Adults with Tardive Dyskinesia,” Expert Review of Clinical Pharmacology 11(3): 209-217; Solmi, M., Pigato, G., Kane, J. M., and Correll, C. U. 2018. “Treatment of Tardive Dyskinesia with VMAT-2 [vesicular monoamine transporter-2] Inhibitors: A Systematic Review and Meta-analysis of Randomized Controlled Trials,” Drug Design, Development, and Therapy 12:1215-1238; Stroup. T. S. and Gray, N. 2018. “Management of Common Adverse Effects of Antipsychotic Medications,” World Psychiatry 17(3): 341-356; Tourna, K. T. B. and Scarff, J. R. 2018. “Valbenazine and Deutetrabenazine for Tardive Dyskinesia,” Innovations in Clinical Neuroscience 15(5-6): 13-16; Ward, K. M. and Citrome, L. 2018. “Antipsychotic-related Movement Disorders: Drug-induced Parkinsonism vs. Tardive Dyskinesia-Key Differences in Pathophysiology and Clinical Management,” Neurology and Therapy 7(2): 233-248; Rasmussen, S. A., Rosebush, P., and Mazurek, M. F. 2017. “The Relationship between Early Haloperidol Response and Associated Extrapyramidal Side Effects,” Journal of Clinical Psychopharmacology 37(1): 8-12; Salem, H., Nagpal, C., Pigott, T., and Teixeira, A. L. 2017. “Revisiting Antipsychotic-induced Akathisia: Current Issues and Prospective Challenges,” Current Neuropharmacology 15(5): 789-798; Osman, M. and Devadas, V. 2016. “Clozapine-induced Dysphagia with Secondary Substantial Weight Loss,” Online, British Medical Journal Case Reports 2016. pii: ber2016216445; Manfredi, G., Solfanelli, A., Dimitri, G., Cuomo, I., Sani, G., Kotzalidis, G. D., and Girardi, P. 2013. “Risperidone-induced Leukopenia: A Case Report and Brief Review of Literature,” General Hospital Psychiatry 35(1): 102.e3-e6; Kane, J. M., Fleischhacker, W. W., Hansen, L., Perlis, R., Pikalov, A. 3rd, and Assunção-Talbott, S. 2009. “Akathisia: An Updated Review Focusing on Second-generation Antipsychotics,” Journal of Clinical Psychiatry 70(5): 627-643).

For direct targeting, drugs which undergo a significant posthepatic and postrenal first pass effect must be processed into the final metabolized form prior to administration. The direct delivery into the internal carotids of antidementia, antipsychotic, and anticonvulsive drugs, for example, not only allows preventing nontargeted tissue from exposure to the drug and its side effects, but increased dosage to the brain of drugs already in use, such as statins, may reveal improved function in yielding more favorable outcomes than does dispersion through the circulation. Direct delivery to the brain in less restricted doses with timely administration allow the potential of drugs already in use to be tested (Kern, D. M., Cepeda, M. S., Lovestone, S., and Seabrook, G. R. 2019. Op cit. section 2a).

The hypotension and gastrointestinal side effects of levodopa for the treatment of Parkinson's disease likely originate in other parts of the brain, so that the direct delivery into the internal carotids of levodopa and dopamine agonists might or might not lessen the vomiting, nausea, and loss of appetite as side effects of levodopa. Since delivery is direct to the brain, there is no need for a DOPA decarboxylase inhibitor to increase the bioavailability and half-life of levodopa inside the brain, and prevent the conversion of L-DOPA into dopamine outside the brain in the systemic circulation where it can cause adverse side effects (Salat, D. and Tolosa, E. 2013. “Levodopa in the Treatment of Parkinson's Disease: Current Status and New Developments,” Journal of Parkinson's Disease (Amsterdam, North Holland, Netherlands) 3(3): 255-269).

More highly resolved targeting of levodopa and dopamine agonists into the pars compacta of the substantia nigra as the origin of the disorder with the object of eradicating symptoms that arise outside these, is through the bilateral insertion of fine catheters into the pars compacta to release levodopa and dopamine agonists. Fixation of the fine drug-feeding catheters in position is microsurgical, the use of tiny nonjacketing side-entry connectors contingent upon the avoidance from anchoring needle insertion of any consequences more serious than the condition to be ameliorated and not spontaneously, according to the tolerance of the patient, resolving over a relatively brief interval. To expedite healing of the wounds caused by the needles, the direct release into the pars compacta of levodopa and dopamine agonists is supplemented with neural stem cells.

The eventual place of vascular servovalves in this is to adjust the dose of dopamine in accordance with sensor reported data to the implanted microcontroller or microprocessor concerning its level, much of the underlying technology remaining under development (Vasudevan, S., Kajtez, J., Bunea, A. I., Gonzalez-Ramos, A., Ramos-Moreno, T., and 6 others 2019. “Leaky Optoelectrical Fiber for Optogenetic Stimulation and Electrochemical Detection of Dopamine Exocytosis from Human Dopaminergic Neurons,” Advanced science (Weinheim, Baden-Württemberg, Germany) 6(24): 1902011; Patriarchi, T., Cho, J. R., Merten, K., Howe, M. W., Marley, A., and 12 others 2018. “Ultrafast Neuronal Imaging of Dopamine Dynamics with Designed Genetically Encoded Sensors,” Science 360(6396). pii: eaat4422; Sun, F., Zeng, J., Jing, M., Zhou, J., Feng, J., and 16 others 2018. “A Genetically Encoded Fluorescent Sensor Enables Rapid and Specific Detection of Dopamine in Flies, Fish, and Mice,” Cell 174(2): 481-496.e19).

For prescription regimen compliance-undependable patients, an implanted automatic disorder response system assures the direct and timely delivery of remedial drugs. Currently, Alzheimer's dementia can be no more than retarded in its progress; however, other forms of dementia appear to be reversible. An implanted automatic disorder response system requires sensors to recognize analytes associated with the type dementia, briefly addressed above in section 2a.

Depression, metabolic and endocrinal conditions, alcohol or drug abuse, and brain lesions can mimic dementia, which reversed may then lead to the misconconception that a dementia had been cured (Tripathi, M. and Vibha, D. 2009. “Reversible Dementias,” Indian Journal of Psychiatry 51 Supplement 1: S52-S55)(see also Santiago-Bravo, G., Sudo, F. K., Assuncao, N., Drummond, C1., and Mattos, P. 2019. “Dementia Screening in Brazil: A Systematic Review of Normative Data for the Mini-mental State Examination,” Clinics (Sao Paulo, Brazil) 74: e971; Sakata, N. and Okumura, Y. 2018. “Thyroid Function Tests before Prescribing Anti-dementia Drugs: A Retrospective Observational Study,” Clinical Interventions in Aging (Auckland, New Zealand) 13:1219-1223; Rodrigues Neto, E., Fonseca, M. K., Guedes, A. C. B., Oliveira, F. H., Hilbig, A., and Fernandez, L. L. 2017. “Neuropathological Findings in Entorhinal Cortex of Subjects Aged 50 Years of Older and Their Correlation with Dementia in a Sample from Southern Brazil,” Dementia and Neuropsychologia (Sao Paulo, Brazil) 11(1): 24-31; Kang, H., Zhao, F., You, L., Giorgetta, C., D. V., Sarkhel, S., and Prakash, R. 2014. “Pseudo-dementia: A Neuropsychological Review,” Annals of the Indian Academy of Neurology (Mumbai [Bombay], India) 17(2): 147-154; Stella, F. 2013. “Assessment of Neuropsychiatric Symptoms in Dementia: Toward Improving Accuracy,” Dementia and Neuropsychologia (Sao Paulo, Brazil) 7(3): 244-251; Piovesana, M. C. F. S., Garcia, F. R., Carrasco, K. G., and Tognola, W. A. 2012. “Reassessment of the Dementia Diagnosis of Alzheimer's Disease in Patients Enrolled on the Cholinesterase Inhibitors Dispensation Program,” Dementia and Neuropsychologia (São Paulo, Brazil) 6(4): 270-275; Bottino, C. M. C., de Padua, A. C., Smid, J., Areza-Fegyveres, R., Novaretti, T., and Bahia, V. S. 2011. “Differential Diagnosis between Dementia and Psychiatric Disorders: Diagnostic Criteria and Supplementary Exams. Recommendations of the Scientific Department of Cognitive Neurology and Aging of the Brazilian Academy of Neurology,” Dementia and Neuropsychologia (São Paulo, Brazil) 5(4): 288-296; Frota, N. A. F., Nitrini, R., Damasceno, B. P., Forlenza, O. V., Dias-Tosta, E., and 3 others 2011. “Criteria for the Diagnosis of Alzheimer's Disease: Recommendations of the Scientific Department of Cognitive Neurology and Aging of the Brazilian Academy of Neurology,” Dementia and Neuropsychologia (São Paulo, Brazil) 5(3): 146-152).

Albeit infrequently, some dementias do appear to be at least partially reversible (see, for example, Day, G. S. 2019. “Reversible Dementias,” Contimaum (Minneapolis, Minnesota) 25(1): 234- 253; Little, M. O. 2018. “Reversible Dementias,” Clinics in Geriatric Medicine 34(4): 537-562; Djukic, M., Wedekind, D., Franz, A., Gremke, M., and Nau, R. 2015. “Frequency of Dementia Syndromes with a Potentially Treatable Cause in Geriatric In-patients: Analysis of a 1-year Interval,” European Archives of Psychiatry and Clinical Neuroscience 265(5): 429-438; Bello, V. M. E. and Schultz, R. R. 2011. “Prevalence of Treatable and Reversible Dementias: A Study in a Dementia Outpatient Clinic,” Dementia and Neuropsychologia (São Paulo, Brazil) 5(1): 44-47).

The cause of ischemic stroke, the ischemia-free elimination of obstruction from the internal carotids is itself addressed in section 2b(2)(d), entitled Anoxia-free aortic repair: aortic prostheses thoracic, thoracoabdominal, and abdominal, and may be essential before the direct delivery into the internal carotids of neuropsychiatric medication can be initiated. Current approaches to the treatment of carotid obstruction consist primarily of removing the plaque through a carotid endarterectomy or crushing it up against the intima through balloon angioplasty, then stenting it to prevent debris from thromboembolizing and interfering with or completely cutting off patency of the internal carotid as would cause a cerebral infarction with consequent neurocognitive and motor impairment and a high risk of death. A more recent transluminal technique that avoids the passage of debris toward the brain by temporarily reversing the direction of blood flow is also addressed below.

Here will be presented a bypass appliance for carotid endarterectomy that provides a dry field with little if any risk of ischemia. Using the means to be described, not only is the open endarterectomy ischemia-free but the bypass can be left in place indefinitely as a prosthesis. The diseased native bifurcation can then be excised, or if left in place, the servovalves can be adjusted to divert a proportion of flow through the native bifurcation and the balance through the bypass. This can proceed automatically, the implanted microcontroller effecting dynamic adjustment of the relative proportion diverted through the bypass on the basis of sensor inputs. This arrangement affords immediate adjustment to counteract hyperperfusion or hypertension.

Both midprocedural hypoperfusion and hyperperfusion just before closure produce postprocedural complications. Midprocedural stroke is not essential to result in the impairment of cognitive function. Accordingly, an important advantage in the use of the bypass shown is that perfusion past the carotid bifurcation is unaffected. Ischemia and hypoperfusion virtually equates to a postoperative decline in cognitive function for three months. Several researchers have noted that post-endarterectomy decline is associated with asymmetric blood flow to the right and left cerebral hemispheres during the procedure.

Use of the device described in this section and section 6 eliminates side to side asymmetrical flow. Whether bypassing the incision in the carotid or passing flow into the external carotid, conventional shunting and clamping is fundamentally different than the bypass described below in section 6, entitled Description of the Preferred Embodiments of the Invention. Also contributing to the prevention of postoperative cognitive impairment are the avoidance of general anesthesia and surgical pneumotaxis through use of short-term mechanical ventilation or a surgical chest dome as described in sections 2a, 2b(2)(k), and 6.

In some cases, flow from the common into the external carotid can be allowed to continue throughout the procedure, only the internal carotid then bypass-fed from the common carotid (see, for example, Robison, T. R., Heyer, E. J., Wang, S., Caccappolo, E., Mergeche, J. L., Shah, S. S., and Connolly, E. S. Jr. 2019. “Easily Screenable Characteristics Associated with Cognitive Improvement and Dysfunction after Carotid Endarterectomy,” World Neurosurgery 121: e200-e206; Traenka, C., Engelter, S. T., Brown, M. M., Dobson, J., Frost. C., and Bonati, L. H. 2019. “Silent Brain Infarcts on Diffusion-weighted Imaging after Carotid Revascularisation: A Surrogate Outcome Measure for Procedural Stroke? A Systematic Review and Meta-analysis,” European Stroke Journal (London, England) 4(2): 127-143; Do. W., Cho, A. R., Kim, E. J., Kim, H. J., Kim, E., and Lee, H. J. 2018. “Ultrasound-guided Superficial Cervical Plexus Block under Dexmedetomidine Sedation versus General Anesthesia for Carotid Endarterectomy: A Retrospective Pilot Study,” Yeungnam University Journal of Medicine 35(1): 45-53; Lin, T., Lai, Z., Lv. Y., Qu, J., Zuo. Z., and 5 others 2018. “Effective Collateral Circulation May Indicate Improved Perfusion Territory Restoration after Carotid Endarterectomy,” European Radiology 28(2): 727-735; Patelis, N., Diakomi, M., Maskanakis, A., Maltezos, K., Schizas, D., and Papaioannou, M. 2018. “General versus Local Anesthesia for Carotid Endarterectomy: Special Considerations,” Online, Saudi Journal of Anaesthesia (Mumbai [Bombay], India) 12(4): 612-617; Zhang, H. P., Ma, X. D., Chen, L. F., Yang, Y., Xu, B. N., and Zhou, D. B. 2016. “Cognitive Function after Carotid Endarterectomy: Early Decline and Later Recovery,” Turkish Neurosurgery 26(6): 833-839; Bennett, K. M., Scarborough, J. E., Cox, M. W., and Shortell. C. K. 2015. “The Impact of Intraoperative Shunting on Early Neurologic Outcomes after Carotid Endarterectomy,” Journal of Vascular Surgery (New York, New York) 61(1): 96-102; Heyer, E. J., Mergeche, J. L., Wang, S., Gaudet, J. G., and Connolly, E. S. 2015. “Impact of Cognitive Dysfunction on Survival in Patients with and without Statin Use following Carotid Endarterectomy,” Neurosurgery 77(6): 880-887; AbuRahma, A. F., Mousa, A. Y., Stone, P. A., Hass. S. M., Dean, L. S., and Keiffer, T. 2011. “Correlation of Intraoperative Collateral Perfusion Pressure during Carotid Endarterectomy and Status of the Contralateral Carotid Artery and Collateral Cerebral Blood Flow,” Annals of Vascular Surgery 25(6): 830-836; Wilson, D. A., Mocco, J., D'Ambrosio, A. L., Komotar, R. J., Zurica, J., and 6 others 2008. “Post-carotid Endarterectomy Neurocognitive Decline is Associated with Cerebral Blood Flow Asymmetry on Post-operative Magnetic Resonance Perfusion Brain Scans,” Neurological Research 30(3): 302-306).

Arterial bifurcations are especially susceptible to atherosclerotic degeneration, and the carotid bifurcation most of all, so that specification of the carotid bifurcation and the prosthesis set forth for its treatment should be understood as exemplary. Addressed above in section 2b(2)(c), entitled Vascular Segment Prostheses: Inline Coupling Jackets and Bridging, or Tie-lines, similar means apply to bypassing straddleable aneurysms, dissections, fistulae, coarctations, and interruptions of the aorta, for example, where, however, there is no branching as necessitates a tubing Y-joint or successive jackets on the common carotid to divert flow to its branches.

Conventional shunting during carotid surgery is old but of questionable benefit (see, for example, Lee, J., Lee, S., Kim, S. W., and Chang, J. W. 2018. “Selective Shunting Based on Dual Monitoring with Electroencephalography and Stump Pressure for Carotid Endarterectomy,” Vascular Specialist International 34(3): 72-76; Cho, J. W., Jeon, Y. H., and Bae, C. H. 2016. “Selective Carotid Shunting Based on Intraoperative Transcranial Doppler Imaging during Carotid Endarterectomy: A Retrospective Single-center Review,” Korean Journal of Thoracic and Cardiovascular Surgery 49(1): 22-28; Bennett, K. M., Scarborough, J. E., Cox, M. W., and Shortell, C. K. 2015. “The Impact of Intraoperative Shunting on Early Neurologic Outcomes after Carotid Endarterectomy,” Journal of Vascular Surgery (New York, New York) 61(1): 96-102; Hans, S. S. and Catanescu, 1. 2015. “Selective Shunting for Carotid Endarterectomy in Patients with Recent Stroke,” Journal of Vascular Surgery (New York, New York) 61(4): 915-919; Cho, J., Lee, K. K., Yun, W. S., Kim, H. K., Hwang, Y. H., and Huh, S. 2013. “Selective Shunt during Carotid Endarterectomy Using Routine Awake Test with Respect to a Lower Shunt Rate,” Journal of the Korean Surgical Society 84(4): 238-244; Kim, T. Y., Choi, J. B., Kim, K. H., Kim, M. H., Shin, B. S., and Park, H. K. 2012. “Routine Shunting is Safe and Reliable for Cerebral Perfusion during Carotid Endarterectomy in Symptomatic Carotid Stenosis,” Korean Journal of Thoracic and Cardiovascular Surgery 45(2): 95-100; Aburahma, A. F., Mousa, A. Y., and Stone, P. A. 2011. “Shunting during Carotid Endarterectomy,” Journal of Vascular Surgery (New York, New York) 54(5): 1502-1510).

However, here the object is to provide a prosthesis used not only to prevent ischemia or stroke during the procedure, but if necessary or beneficial, left in place as a prosthesis to the end of life with the capability for automatic pipeline targeted drug delivery directly to the treatment site. Hardly limited to the carotid bifurcation, bypass with built in means for drug delivery applies to all arterial bifurcations affected by atherosclerosis. To assure that blood flow through the carotids is unaffected during the endarterectomy, the diversion valves on the common carotid and its branches must actuate simultaneously from a common switch as in a sudden compound vascular bypass, or sudden switched-organ transplantation.

When the Y-bypass will remain as a prosthesis, unidirectional or set-once solenoid valves are used. When the bypass is to be removed, reversible solenoid valves are used. Even when the bypass is removed, the valve on the common carotid is best left in place to allow continued use of its accessory channel or channels. The accessory channels in the diversion valves used to connect the bypass allow the direct pipeline targeting of drugs through and downstream to the treated segment.

The conventional procedure risks hypoxia and does so most likely in an elderly patient least likely to tolerate it. Bypass using the means to be described not only allows the direct targeting drugs to the treatment site periprocedurally and long thereafter automatically, but the side-entry bypass diversion servovalves can adjust the volume of flow to avert hyperperfusion or hypertension. Allowing the native bifurcation to remain rather than be excised following endarterectomy offers advantages in allowing the adjustment of flow.

Complete closure of the one servovalve on the common carotid completely closes off inflow and one each on the internal and external carotids outflow through the native bifurcation. As addressed in section 2b(2)(b), in exceptional cases, a proportion of the flow through the bypass can be diverted into another vessel. Drugs delivered with the valves retracted so that the substrate lumen is completely open flow through the affected segment. The release of drugs thus is controlled automatically by the implanted microprocessor or microcontroller, assuring adherence in an incompetent patient. Closing either the valve on the internal or external carotid allows blood to flow through the other.

The schemes depicted herein in FIGS. 25A and 25, allow targeting drugs to the brain through jackets applied to the right and/or left internal carotid. With the diversion valves closed to bypass a lesioned or extirpated section, the bypass can remain in place indefinitely as a prosthesis. Prepositioned so that it is directly accessible for drug-targeting without entry from outside the body, virtually any medicinal, hence, any postoperative complication, such as restenosis, in-stent restenosis, or an accumulation of clot can be prevented or reversed. Restricted flow through the bypass, for example, is readily detected by thin film sensors along the tube lining which signal the microcontroller or master control microprocessor to initiate the release of a thrombolytic.

Sensors can also be provided to continuously detect hypocarbia presaging vasoconstriction, increased cerebrovascular resistance with cerebral ischemia, and measure the blood pressure during and following the procedure. Complications that might arise mid- and/or postprocedurally include thromboembolic cerebral and myocardial infarction, arterial spasm. Postprocedural complications include cervical hematoma and airway compromise (see, for example, Paciaroni, M., Eliasziw. M., Kappelle, L. J., Finan, J. W., Ferguson, G. G., and Barnett, H. J. 1999. “Medical Complications Associated with Carotid Endarterectomy. North American Symptomatic Carotid Endarterectomy Trial (NASCET),” Stroke 30(9): 1759-1763).

Accordingly, such bypasses can be used in conjunction with conventional surgical correction, or if the native tissue was not salvaged due, for example, to Ehlers-Danlos or Loeys-Dietz syndrome, or after having been resected as cancerous, for example, left in place as prostheses (see, for example, Kim, S. T., Brinjikji, W., Lanzino, G., and Kallmes, D. F. 2016. “Neurovascular Manifestations of Connective-tissue Diseases: A Review,” Interventional Neuroradiology 22(6): 624-637; Chassin-Trubert Contreras, A. M. 2013. “Carotid Dissection Associated with Ehlers-Danlos Syndrome. Report of One Case,” (in Spanish with abstract at Pubmed), Revista Medica de Chile [Chilean Medical Journal] 141(3): 392-395; Dohle, C. and Baehring, J. M. 2012. “Multiple Strokes and Bilateral Carotid Dissections: A Fulminant Case of Newly Diagnosed Ehlers-Danlos Syndrome Type IV,” Journal of the Neurological Sciences 318(1-2): 168-170).

Other means such as linear and rotary atherectomizers are available but also present complications and are used less frequently. Here, depending upon the condition of the patient and a large number of medical considerations, the bypass is:
1. Applied during the endarterectomy and possibly stenting, after which the bypass is removed.
2. Applied during the carotid endarterectomy, after which the bypass is not removed but allowed to remain attached by the connecting diversion valves which can be closed to bypass or opened to not bypass the carotid bifurcation as necessary.
3. Not removed, the diversion valves closed to bypass the defective segment whether left or removed.

The decision to remove or leave such a bypass in place permanently as a prosthetic replacement for an irreversibly diseased or damaged native vessel can be made during or upon completion of the endarterectomy. Pathogen and thrombus-resistant coatings too short-lived for permanent implants, jackets and valve-jackets used to connect the bypass to the substrate vessel for permanent implantation are provided with accessory channels. The valves or tiny pumps at the outlets of the small flat fluid drug reservoirs of the disorder response system positioned subcutaneously in the pectoral region can be actuated by the implanted microcontroller, or in a hierarchical control system to treat comorbidities, the microprocessor to release doses of drugs on schedule in accordance with the control prescription-program automatically.

Complications during and following a conventional endarterectomy are primarily due to ischemia and thromboembolism. The bifurcation is at once the most common site of obstruction, and the internal carotid supplying the brain, an embolism will likely cause a cerebral infarction. The synthetic material of the bypass is little susceptible to the accumulation of clot, and the valves incorporate accessory channels through which an anticoagulant can be directly targeted to the site.

When the native carotids are severely damaged due to disease or accidental trauma as would necessitate reconstruction, the Y-bypass is left in place as a prosthesis and the native vessels removed, thus eliminating the need to harvest other vessels with the risk of complications this poses when time will not allow generating autologous tissue engineered vessels. Since the Y-bypass diversion valve on the common carotid incorporates at least one accessory channel, or drugline, to deliver drugs through the bifurcation, the prosthesis makes possible directly pipeline targeted follow-up care. U.S. Pat. No. 11,389,171, entitled Integrated System for the Infixion and Retrieval of Implants, provides two methods with means for restricting a drug to the target-through the release of a reversal agent through a side-entry jacket into the venous drainage, or the extraction of the substance by a magnetized jacket from the venous drainage by attraction of the superparamagnetic carrier to which the neutralizing agent is bonded. Where the drug, such as one too highly radioactive to be released must be completely eradicated from continued passage through the circulatory system, both can be applied, with radiation shielded lines and jackets used.

Both carotid occlusion and Alzheimer's disease, for example, often concurrent in the elderly, an anticoagulant can be chosen to not pass the blood brain barrier for concerns of causing a brain hemorrhage or released through a valve jacket accessory channel in one or both internal carotids, to cross the barrier in the expectation that this will either deter the development of reduce the severity of Alzheimer's disease, for example (see, for example, Benseny-Cases, N., Karamanos, T. K., Hoop, C. L., Baum, J., and Radford, S. E. 2019. “Extracellular Matrix Components Modulate Different Stages in β2-microglobulin Amyloid Formation,” Journal of Biological Chemistry 294(24): 9392-9401; Mycroft-West, C. J., Cooper, L. C., Devlin, A. J., Procter, P., Guimond, S. E., and 5 others 2019. “A Glycosaminoglycan Extract from Portunas pelagicus Inhibits BACE1 [beta-secretase enzyme 1], the β Secretase Implicated in Alzheimer's Disease,” Online, Marine Drugs MDPI [Multidisciplinary Digital Publishing Institute] International Journal of Molecular Sciences (Basel, Switzerland) 17(5). pii: E293; Yan, Y., Bejoy, J., Marzano, M., and Li, Y. 2019. “The Use of Pluripotent Stem Cell-derived Organoids to Study Extracellular Matrix Development during Neural Degeneration,” Cells 8(3). pii: E242; Bejoy, J., Song, L., Wang, Z., Sang, Q. X., Zhou, Y., and Li, Y. 2018. “Neuroprotective Activities of Heparin, Heparinase III, and Hyaluronic Acid on the Aβ42-treated Forebrain Spheroids Derived from Human Stem Cells,” American Chemical Society Biomaterials Science and Engineering 4(8): 2922-2933; Bejoy, J., Wang, Z., Bijonowski, B., Yang, M., Ma, T., Sang, Q. X., and Li, Y. 2018. “Differential Effects of Heparin and Hyaluronic Acid on Neural Patterning of Human Induced Pluripotent Stem Cells,” American Chemical Society Biomaterials Science and Engineering (12): 4354-4366; Stewart, K. L., Hughes, E., Yates, E. A., Middleton, D. A., and Radford, S. E. 2017. “Molecular Origins of the Compatibility between Glycosaminoglycans and Aβ40 Amyloid Fibrils,” Journal of Molecular Biology 429(16): 2449-2462; Stewart, K. L., Hughes, E., Yates, E. A., Akien, G. R., Huang, T. Y., and 6 others 2016. “Atomic Details of the Interactions of Glycosaminoglycans with Amyloid-β Fibrils,” Journal of the American Chemical Society 138(27): 8328-8331; Bergamaschini, L., Rossi, E., Vergani, C., and De Simoni, M. G. 2009. “Alzheimer's Disease: Another Target for Heparin Therapy,” Scientific World Journal 9:891-908). Patey, S., Edwards, E. A., Yates, E. A., and Tumbull, J. E. 2008. “Engineered Heparins: Novel Beta-secretase Inhibitors as Potential Alzheimer's Disease Therapeutics,” Neurodegenerative Diseases (Basel, Switzerland) 5(3-4): 197-199; Ma, Q., Cornelli, U., Hanin, I., Jeske, W. P., Linhardt, R. J., and 3 others 2007. “Heparin Oligosaccharides as Potential Therapeutic Agents in Senile Dementia,” Current Pharmaceutical Design 13(15): 1607-1616).

Carotid endarterectomy usually responds to a buildup of plaque at the carotid bifurcation. Stroke prevention is safest using the bypass shown in FIG. 25A and possibly a surgical chest dome described below in section 2b(2)(k), entitled Surgical Chest Dome, and section 6, entitled Description of the Preferred Embodiments of the Invention. Upon completion of the endarterectomy, the druglines and sensors are best left in position to allow the continued automatic targeting of medication. Tumors that emerge at the base of the heart (heart-base tumors, chemodectomas) may also occlude the carotids.

Conditions affecting carotid patency are addressed above in this section. Also appearing in the carotids may be carotid body tumors, rarely, glomus, or glomus cell tumors, and nonchromaffin tumors, or paragangliomas, which are, however, akin to chromaffin-positive pheochromocytomas. Other conditions that may affect the carotids necessitating use of the bypasses shown in FIG. 25A or 25B include a carotid sinus that is dilated as well as atheromatous, stenosis, or a reduction in luminal cross-section, contralateral stenosis, and impaired cerebral autoregulation as predisposes to postsurgical and post-stroke hyperperfusion (Kirchoff-Torres and Bakradze cited below).

As is true of coronary artery bypass grafting versus angioplasty and stenting, severe cases are usually better treated surgically. While infrequent, post-carotid endarterectomy patients should be closely monitored during the month following surgery to determine the need for further treatment to prevent a contralateral stroke. Carotid tumors are well covered in the literature (see, for example, Ikram, A. and Rehman, A. 2024. “Paraganglioma,” Online, Treasure Island, Florida: StatPearls Publishing Co.; Forbes, J. and Menezes, R. G. 2023. “““Anatomy, Head and Neck: Carotid Bodies,” Online, Treasure Island, Florida: StatPearls Publishing Co.; Liu, J., Mu, H., and Zhang, W. 2021. “Diagnosis and Treatment of Carotid Body Tumors,” Online, American Journal of Translational Research (Madison, Wisconsin) 13(12): 14121-14132; Ifeoluwa, A., Lazar, I., Szovordi, E., and Karosi, T. 2017. “Management of Carotid Body Tumor in Pediatric Patients: A Case Report and Review of the Literature,” International Journal of Pediatric Otorhinolaryngology (Limerick, Ireland) 93:47-52).

As does surgery in general, conventional carotid endarterectomy poses a risk of cerebral ischemia, which if brief, is experienced as temporary ischemic attacks, and if not, as a stroke leading to a cerebral infarction, the incidence highest for cardiac and major vascular surgery, and the trigger often cardioembolism upon cessation of medication (see, for example, Kummer B. R., Hazan, R., Merkler, A. E., Kamel, H., Willey, J. Z., and 5 others 2020. A Multilevel “Analysis of Surgical Category and Individual Patient-level Risk Factors for Postoperative Stroke,” Neurohospitalist (Thousand Oaks, California) 10(1): 22-28; Manning, M. W., Kumar, P. A., Maheshwari, K., and Arora, H. 2020. “Post-reperfusion Syndrome in Liver Transplantation—An Overview,” Journal of Cardiothoracic and Vascular Anesthesia 34(2): 501-511; Fisher, O., Benson, R. A., Tian, F., Dale, N. E., and Imray, C. H. 2019. “Purine Nucleoside Use as Surrogate Markers of Cerebral Ischaemia during Local and General Anaesthetic Carotid Endarterectomy,” SAGE Open Medicine 7:2050312119865120; Imai. T., Izumi, T., Isoda, H., Ishiguro, K., Mizuno, T., and 7 others 2019. “Factors Influencing Blood Flow Resistance from a Large Internal Carotid Artery Aneurysm Revealed by a Computational Fluid Dynamics Model,” Nagoya Journal of Medical Science 81(4): 629-636; Andereggen, L., Amin-Hanjani, S., El-Koussy, M., Verma, R. K., Yuki, K., and 9 others 2018. “Quantitative Magnetic Resonance Angiography as a Potential Predictor for Cerebral Hyperperfusion Syndrome: A Preliminary Study,” Journal of Neurosurgery 128(4): 1006-1014; Erdoes, G., Rummel, C., Basciani, R. M., Verma, R., Carrel, T., and 3 others 2018. “Limitations of Current Near-infrared Spectroscopy Configuration in Detecting Focal Cerebral Ischemia during Cardiac Surgery: An Observational Case-series Study,” Artificial Organs 42(10): 1001-1009; Ghebremichael, S., Sridhar, S., Guzman-Reyes, S., and Pivalizza, E. G. 2018. “Dramatic Change in Cerebral Oximetry during Liver Transplantation,” Proceedings Baylor University Medical Center 31(2): 185-186; Hsieh, C. Y., Huang, C. W., Wu, D. P., and Sung, S. F. 2018. “Risk of Ischemic Stroke after Discharge from Inpatient Surgery: Does the Type of Surgery Matter?,” Public ibrary of Science One 13(11): e0206990; Kirchoff-Torres, K. F. and Bakradze, E. 2018. “Cerebral Hyperperfusion Syndrome after Carotid Revascularization and Acute Ischemic Stroke,” Current Pain and Headache Reports 22(4): 24; Ko. S. B. 2018. “Perioperative Stroke: Pathophysiology and Management,” Korean Journal of Anesthesiology 71(1): 3-11; Müller, M. D., von Felten, S., Algra, A., Becquemin. J. P., Brown, M.5., and 13 others 2018. “Immediate and Delayed Procedural Stroke or Death in Stenting versus Endarterectomy for Symptomatic Carotid Stenosis,” Stroke 49(11): 2715-2722; Thudium, M., Heinze, I., Ellerkmann, R. K., and Hilbert, T. 2018. “Cerebral Function and Perfusion during Cardiopulmonary Bypass: A Plea for a Multimodal Monitoring Approach,” Heart Surgery Forum 21(1): E028-E035; Clouse, W. D., Ergul. E. A., Patel, V. I., Lancaster, R. T., LaMuraglia, G. M., Cambria, R. P., and Conrad, M. F. 2017. “Characterization of Perioperative Contralateral Stroke after Carotid Endarterectomy,” Journal of Vascular Surgery (New York, New York) 66(5): 1450-1456; Grau, A. J., Eicke, M., Burmeister, C., Hardt, R., Schmitt, E., and Dienlin, S. 2017. “Risk of Ischemic Stroke and Transient Ischemic Attack is Increased up to 90 Days after Non-carotid and Non-cardiac Surgery,” Cerebrovascular Disease 43(5-6): 242-249; Hinojosa, C. A., Anaya-Ayala, J. E., Laparra-Escareno, H., Torres-Machorro, A., Lizola, R., and Gamboa-Dominguez, A. 2017. “Concomitant Surgical Treatment of Symptomatic Carotid Artery Disease with a Coexisting Shamblin I Carotid Body Tumor,” Vascular and Endovascular Surgery 51(2): 103-107; Wang, G. J., Beck, A. W., DeMartino, R. R., Goodney, P. P., Rockman, C. B., and Fairman, R. M. 2017. “Insight into the Cerebral Hyperperfusion Syndrome following Carotid Endarterectomy from the National Vascular Quality Initiative,” Journal of Vascular Surgery (New York, New York) 65(2): 381-389.e2; Budincevic, H., Piršic, A., Bohm, T., Trajbar. T., Ivkosic. A., and 3 others 2016. “Carotid Body Tumor as a Cause of Stroke,” Internal Medicine (Tokyo, Japan) 55(3): 295-298; Farooq, M. U., Goshgarian, C., Min, J., and Gorelick, P. B. 2016. “Pathophysiology and Management of Reperfusion Injury and Hyperperfusion Syndrome after Carotid Endarterectomy and Carotid Artery Stenting,” Online, BioMed Central Experimental and Translational Stroke Medicine (London, England) 8(1): 7; Li, J., Shalabi, A., Ji, F., and Meng, L. 2016. “Monitoring Cerebral Ischemia during Carotid Endarterectomy and Stenting,” Journal of Biomedical Research 31(1): 11-16; Sorensen, H., Grocott, H. P., and Secher, N. H. 2016. “Near Infrared Spectroscopy for Frontal Lobe Oxygenation during Non-vascular Abdominal Surgery,” Clinical Physiology and Functional Imaging 36(6): 427-435; Sorensen, H., Nielsen, H. B., and Secher, N. H. 2016. “Near-infrared Spectroscopy Assessed Cerebral Oxygenation during Open Abdominal Aortic Aneurysm Repair: Relation to End-tidal CO2 Tension,” Journal of Clinical Monitoring and Computing 30(4): 409-415; Vlisides, P. and Mashour, G. A. 2016. “Perioperative Stroke,” Canadian Journal of Anaesthesia 63(2): 193-204; Sorensen, H., Grocott, H. P., Niemann, M., Rasmussen, A., Hillingso, J. G., Frederiksen, H. J., and Secher, N. H. 2014. “Ventilatory Strategy during Liver Transplantation: Implications for Near-infrared Spectroscopy-determined Frontal Lobe Oxygenation,” Online, Frontiers in Physiology 5:321; Kim, K. H., Lee, C. H., Son, Y. J., Yang, H. J., Chung, Y. S., and Lee, S. H. 2013. “Post-carotid Endarterectomy Cerebral Hyperperfusion Syndrome: Is It Preventable by Strict Blood Pressure Control?,” Journal of the Korean Neurosurgical Society 54(3): 159-163; Lieb, M., Shah, U., and Hines, G. L. 2012. “Cerebral Hyperperfusion Syndrome after Carotid Intervention: A Review,” Cardiology in Review 20(2): 84-89; Şanlι, A, Öz, K., Ayduran, E, Aydιn S. Altιn, G., and Eken, M. 2012. “Carotid Body Tumors and Our Surgical Approaches,” Indian Journal of Otolaryngology and Head and Neck Surgery 64(2): 158-161).

In an undisturbed and rational adult patient, use of the vascular valve-based carotid bifurcation bypass device addressed in sections 2b(2)(d) and 6 allows surgical endarterectomy under regional if not local anesthesia. This leaves the patient conscious, allowing immediate awareness of a mid- or postprocedural stroke when it occurs, so that a thrombolytic such as a recombinant tissue plasminogen activator, streptokinase, or anistreplase) can be administered immediately rather than after an hour or so when the patient regains consciousness (Ko, S. B. 2018, Op cit., preceding paragraph).

Nevertheless, general anesthesia is often preferred (see, for example, Harky, A., Chan, J. S. K., Kot, T. K. M., Sanli, D., Rahimli, R., and 7 others 2020. “General Anesthesia versus Local Anesthesia in Carotid Endarterectomy: A Systematic Review and Meta-Analysis,” Journal of Cardiothoracic and Vascular Anesthesia 34(1): 219-234; Kim, J. W., Huh, U., Song, S., Sung, S. M., Hong, J. M., and Cho, A. 2019. “Outcomes of Carotid Endarterectomy According to the Anesthetic Method: General versus Regional Anesthesia,” Korean Journal of Thoracic and Cardiovascular Surgery 52(6): 392-399; Dakour Aridi, H., Paracha, N., Nejim, B., Locham, S., and Malas, M. B. 2018. “Anesthetic Type and Hospital Outcomes after Carotid Endarterectomy from the Vascular Quality Initiative Database,” Journal of Vascular Surgery (New York, New York) 67(5): 1419-1428; Hye, R. J., Voeks, J. H., Malas, M. B., Tom, M., Longson, S., Blackshear, J. L., and Brott, T. G. 2016. “Anesthetic Type and Risk of Myocardial Infarction after Carotid Endarterectomy in the Carotid Revascularization Endarterectomy versus Stenting Trial (CREST),” Journal of Vascular Surgery (New York, New York) 64(1): 3-8.e1; Kavakli, A. S. and Ozturk, N. K. 2016. “Anesthetic Approaches in Carotid Body Tumor Surgery,” Northern Clinics of Istanbul 3(2): 97-103; Lee, J., Huh, U., Song. S., Chung, S. W., Sung, S. M., and Cho, H. J. 2016. “Regional Anesthesia with Dexmedetomidine Infusion: A Feasible Method for the Awake Test during Carotid Endarterectomy,” Annals of Vascular Diseases (Tokyo, Japan) 9(4): 295-299; Anwari, J. S. 2015. “Dexmedetomidine during Carotid Endarterectomy: A Review of a Commentary?,” Saudi Journal of Anaesthesia 9(1): 104; Nair, A. S. 2014. “Benefits of Using Dexmedetomidine during Carotid Endarterectomy: A Review,” Saudi Journal of Anaesthesia 8(2): 264-267; Vaniyapong, T., Chongruksut, W., and Rerkasem, K. 2013. “Local versus General Anaesthesia for Carotid Endarterectomy,” Cochrane Database of Systematic Reviews(12): CD000126; Calligaro, K. D. and Dougherty, M. J. 2005. “Correlation of Carotid Artery Stump Pressure and Neurologie Changes during 474 Carotid Endarterectomies Performed in Awake Patients,” Journal of Vascular Surgery (New York, New York) 42(4): 684-689).

Setting aside postprocedural headache and confusion as additional deterrents to the use of general anesthesia, whether the analysis of blood intermediate purine nucleosides adenosine, inosine and hypoxanthine, which rise dramatically upon carotid cross-clamping and the need for shunting, as a midprocedural endarterectomy ischemia or hypoxia biomarker can be depended upon remains inconclusive (Dale, N., Tian, F., Sagoo, R., Phillips, N., Imray, C., and Roffe, C. 2019. “Point-of-care Measurements Reveal Release of Purines into Venous Blood of Stroke Patients,” Purinergic Signalling (Dordrecht, Holland) 15(2): 237-246; Fisher, O., Benson, R. A., and Imray, C. H. 2019. “The Clinical Application of Purine Nucleosides as Biomarkers of Tissue Ischemia and Hypoxia in Humans in Vivo,” Biomarkers in Medicine 13(11): 953-965; Fisher, O., Benson, R. A., Tian, F., Dale, N. E., and Imray, C. H. 2019. “Purine Nucleoside Use as Surrogate Markers of Cerebral Ischaemia during Local and General Anaesthetic Carotid Endarterectomy,” SAGE Open Medicine (London, England) 7:2050312119865120; Martin, A. J., Dale, N., Imray, C. H. E., Roffe, C., Smith, C. J., Tian, F., and Price, C. I. 2019. “The Association between Early Neurological Deterioration and Whole Blood Purine Concentration during Acute Stroke,” Biomarker Research 7:7; Tian, F., Bibi, F., Dale, N., and Imray, C. H. E. 2017. “Blood Purine Measurements as a Rapid Real-time Indicator of Reversible Brain Ischaemia,” Purinergic Signalling (Dordrecht, Holland) 13(4): 521- 528; Szabo, P., Lantos, J., Nagy, L., Keki, S., Volgyi, E., and 3 others 2016. “l-Arginine Pathway Metabolites Predict Need for Intra-operative Shunt during Carotid Endarterectomy,” European Journal of Vascular and Endovascular Surgery 52(6): 721-728; Weigand, M. A., Michel, A., Eckstein, H. H., Martin, E., and Bardenheuer, H. J. 1999. “Adenosine: A Sensitive Indicator of Cerebral Ischemia during Carotid Endarterectomy,” Anesthesiology 91(2): 414-421).

While carotid endarterectomy poses the least risk of midprocedural stroke, transcarotid artery revascularization reduces the risk compared to conventional transluminal or transcatheteric treatment and stenting. Transluminal techniques with the use of run-ahead filters as may be used in conventional angioplasty remains to appear in the literature. Whether transcarotid artery revascularization allows the thoroughness of plaque removal when abundant on a par with open surgery remains to be documented.

Placement of a stent is not tied to angioplasty but can follow surgery as well, where stent positioning is directly and more tightly controlled. Most surgeons are likely to prefer the direct view of the field and manual control provided by open surgery to the indirect view of imaging and remote manipulation. Anatomic or medical high-risk factors for carotid endarterectomy sometimes prompt a preference for transcarotid artery revascularization.

The technique does, however, achieve entry through a small incision that offers less opportunity for microbial intrusion, mitigates cranial nerve injury, and compared to transfemoral carotid artery stenting, is associated with a lower risk of stroke or death (see, for example. Lackey, A. R., Erben, Y., Franco, J. A. D. R., Meschia, J. F., and Lal. B. K. 2020. “Transcarotid Artery Revascularization Results in Low Rates of Periprocedural Neurologic Events, Myocardial Infarction, and Death,” Current Cardiology Reports 22(1): 3; Schermerhom, M. L., Liang, P., Dakour-Aridi, H., Kashyap, V. S., Wang, G. J., and 4 others 2020. “In-hospital Outcomes of Transcarotid Artery Revascularization and Carotid Endarterectomy in the Society for Vascular Surgery Vascular Quality Initiative,” Journal of Vascular Surgery (New York, New York) 71(1): 87-95; Kashyap. V. S., King. A. H., Foteh, M. I., Janko, M., Jim, J., and 4 others 2019. “A Multi-institutional Analysis of Transcarotid Artery Revascularization Compared to Carotid Endarterectomy,” Journal of Vascular Surgery (New York, New York) 70(1): 123-129; Luk, Y., Chan, Y. C., and Cheng, S. W. 2019. “Transcarotid Artery Revascularization as a New Modality of Treatment for Carotid Stenosis,” Annals of Vascular Surgery November 6. pii: S0890-5096(19) 30949-5; Malas, M. B., Dakour-Aridi, H., Wang, G. J., Kashyap. V. S., and Motaganahalli, R. L. 2019. “Transcarotid Artery Revascularization versus Transfemoral Carotid Artery Stenting in the Society for Vascular Surgery Vascular Quality Initiative,” Journal of Vascular Surgery (New York, New York) 69(1): 92-103.e2; Schermerhom, M. L., Liang. P., Eldrup-Jorgensen, J., Cronenwett, J. L., Nolan, B. W., and 4 others 2019. “Association of Transcarotid Artery Revascularization vs Transfemoral Carotid Artery Stenting with Stroke or Death among Patients with Carotid Artery Stenosis,” Journal of the American Medical Association 322(23): 2313-2322; Williams, Z. F., Olivere, L. A., Schroder, J., Cox, M. W., Long. C. A., and Southerland, K. W. 2019. “Simultaneous Transcarotid Artery Revascularization with Flow Reversal and Coronary Artery Bypass Grafting: A Novel Hybrid Technique,” Journal of Vascular Surgery Cases and Innovative Techniques (New York, New York) 5(4): 572-575).

While use of the bypass appliance described herein eliminates clamping, selective patch angioplasty and intraoperative shunting in conventional carotid endarterectomy still require cross-clamping. The elimination of cross-clamping and general anesthesia at endarterectomy avoids the risk of cerebral ischemia, general anesthesia-associated changes, and to the extent that it is attributable to these factors, the risk of postoperative stroke (see, for example, Domenick Sridharan, N., Thirumala, P., Chaer, R., Balzer, J., Long. B., and 3 others 2018. “Predictors of Cross-clamp-induced Intraoperative Monitoring Changes during Carotid Endarterectomy Using Both Electroencephalography and Somatosensory Evoked Potentials,” Journal of Vascular Surgery (New York, New York) 67(1): 191-198; Naylor, A. R., Ricco, J. B., de Borst, G. J., Debus, S., de Haro. J., and 25 others 2018. “Editor's Choice-Management of Atherosclerotic Carotid and Vertebral Artery Disease: 2017 Clinical Practice Guidelines of the European Society for Vascular Surgery (ESVS),” European Journal of Vascular and Endovascular Surgery 55(1): 3-81; Cheng, I., Vyas, K. S., Velaga, S., Davenport, D. L., and Saha, S. P. 2017. “Outcomes of Carotid Endarterectomy with Primary Closure,” International Journal of Angiology 26(2): 83-88; Maertens, V., Maertens, H., Kint. M., Coucke, C., and Blomme, Y. 2016. “Complication Rate after Carotid Endarterectomy Comparing Patch Angioplasty and Primary Closure,” Annals of Vascular Surgery 30:248-252; Kapoor, R., Evins. A. I., Marcus. J., Rigante, L., Kubota, M., and Stieg, P. E. 2015. “Selective Patch Angioplasty and Intraoperative Shunting in Carotid Endarterectomy: A Single-center Review of 141 Procedures,” Online, Cureus (Palo Alto, California) 7(10): e367; Simon, M. V., Chiappa, K. H., Kilbride, R. D., Rordorf, G. A., Cambria. R. P., and 5 others 2012. “Predictors of Clamp-induced Electroencephalographic Changes during Carotid Endarterectomies,” Journal of Clinical Neurophysiology 29(5): 462-467; Rerkasem, K. and Rothwell, P. M. 2009. “Patch Angioplasty versus Primary Closure for Carotid Endarterectomy,” Cochrane Database of Systematic Reviews(4): CD000160; Aleksic, M., Gawenda, M., Heckenkamp, J., Matoussevitch, V., Coburger, S., and Brunkwall. J. 2004. “Prediction of Cerebral Ischemic Tolerance during Carotid Cross-clamping by Angiographic Criteria,” European Journal of Vascular and Endovascular Surgery 27(6): 640-645: Stoneham, M. D. and Wamer, O. 2001. “Blood Pressure Manipulation during Awake Carotid Surgery to Reverse Neurological Deficit after Carotid Cross-clamping,” British Journal of Anaesthesia 87(4): 641-644; Blume, W. T., Ferguson, G. G., and McNeill, D. K. 1986. “Significance of EEG Changes at Carotid Endarterectomy,” Stroke 17(5): 891-897).

Post-endarterectomy closure or permanent bypass using solenoid or servovalve-jackets is unique. Broadly, bypasses using solenoid or servovalve-jackets may be used to allow ischemia-free surgery of the native vessels, or if the vessels are diseased, left in place to bypass these permanently with, moreover, channels that enable the postoperative targeted release of medication from a small port at the body surface directly to the treatment site. In a perinate or infant, depending upon the specific structures involved, the parts of the bypass allow for sufficient growth to avoid replacement for years if not for life. In open vascular surgery of the kinds to which the procedure to be described applies, such as a carotid endarterectomy or aneurysmal repair, side-entry and side-entry diversion jackets make possible bypassing the treatment area with little if any change in the mean arterial pressure for more than the brief time it takes to place, and if not to remain as a permanent prosthesis, remove the jacket device.

Since the field is open, the operator can manually switch one or two diversion jackets from the chute extended diversion to the chute retracted normal flow position. Also, in open surgery, only the port at the body surface is remote. In a solid organ transplant where multiple diversion jackets must be switched, jacket control is by a switch that actuates the chute solenoids simultaneously. The pressure, sheer stress, inducement of turbulence, and other mechanical factors effective through the device should approximate normal values for the patient.

That is, but without susceptibility to stenosis, remodeling, atheroma or other lesion, or if an aneurysm, the dilatation that is the circumstance requiring treatment (see, for example, Abas, A., Mokhtar, N. H., Ishak, M. H., Abdullah, M. Z., and Ho Tian, A. 2016. “Lattice Boltzmann Model of 3D Multiphase Flow in Artery Bifurcation Aneurysm Problem,” Computational and Mathematical Methods in Medicine 2016:6143126; Yuan, C. and Parker, D. L. 2016. “Three-dimensional Carotid Plaque M R Imaging,” Neuroimaging Clinics of North America 26(1): 1-12; Ha, H., Kim, G. B., Kweon, J., Lee, S., Kim, Y. H., Lee, D. H., Yang, D. H., and Kim, N. 2016. “Hemodynamic Measurement Using Four-Dimensional Phase-Contrast MRI: Quantification of Hemodynamic Parameters and Clinical Applications,” Korean Journal of Radiology 17(4): 445-462; Chen, H., Zhang, Q., and Kerwin, W. 2016. “Analysis of Multicontrast Carotid Plaque MR Imaging,” Neuroimaging Clinics of North America 26(1): 13-28; Vesey, A. T., Dweck, M. R., and Fayad, Z. A. 2016. “Utility of Combining PET and MR Imaging of Carotid Plaque,” Neuroimaging Clinics of North America 26(1): 55-68; Pineda Zapata, J. A., Delgado de Bedout, J. A., Rascovsky Ramirez, S., Bustamante, C., Mesa, S., and Calvo Betancur, V. D. 2014. “A Practical Introduction to the Hemodynamic Analysis of the Cardiovascular System with 4D Flow MRI,” Radiologia 56(6): 485-495; van Wijk, D. F., Strang, A. C., Duivenvoorden, R., Enklaar, D. J., van der Geest, R. J., and 4 others 2014. “Increasing Spatial Resolution of 3 T MRI Scanning Improves Reproducibility of Carotid Arterial Wall Dimension Measurements,” MAGMA 27(3): 219-226; Negahdar, M. 2014. 4D Reduced TE [echo time] (RTE) Spiral Phase Contrast MRI for Rapid Quantification and Visualization of Blood, Flow and Hemodynamics, dissertation, Speed School of Engineering, University of Louisville, available at http://ir.library.louisville.edu/cgi/viewcontent.cgi?article=3359&context=etd; Koktzoglou, I. 2013. “4D Dark Blood Arterial Wall Magnetic Resonance Imaging: Methodology and Demonstration in the Carotid Arteries,” Magnetic Resonanace in Medicine 69(4): 956-965; Kadbi. M., Negahdar, M., Traughber, M., Martin, P., and Amini, A. A. 2013. “Assessment of Flow and Hemodynamics in the Carotid Artery Using a Reduced TE [echo time] 4D Flow Spiral Phase-contrast MRI,” Conference Proceedings Institute of Electrical and Electronics Engineers Engineering in Medicine and Biology Society 2013:1100-1103; Mark, M., Frydrychowicz, A., Kozerke, S., Hope, M., and Wieben, O. 2012. “4D Flow MRI,” Journal of Magnetic Resonance Imaging 36(5): 1015-1036; Roche-Nagle, G. and Oreopolous, G. 2009. “Extracranial Carotid Artery Aneurysms,” American Journal of Surgery 197(6): e64-e65; Zhao, S. Z., Xu. X. Y., Hughes, A. D., Thom, S. A., Stanton, A. V., Ariff, B., and Long, Q. 2000. “Blood Flow and Vessel Mechanics in a Physiologically Realistic Model of a Human Carotid Arterial Bifurcation,” Journal of Biomechanics 33(8): 975-984; Milner, J. S., Moore, J. A., Rutt, B. K., and Steinman, D. A. 1998. “Hemodynamics of Human Carotid Artery Bifurcations: computational Studies with Models Reconstructed from Magnetic Resonance Imaging of Normal Subjects,” Journal of Vascular Surgery (New York, New York) 28(1): 143-156).

Avoiding the conventional equipment means that there is little if any loss in continuity or disruption in mean arterial pressure and the adverse sequelae this can precede. While addressed as unilateral, the same information applies on the contralateral side. Disadvantageously elevated while awaiting surgery, blood pressure is advantageously reduced following nonendovascular repair of a thoracic aortic aneurysm or a carotid endarterectomy, for example, (see, for example, Varon, J. and Marik, P. E. 2008. “Perioperative Hypertension Management,” Vascular Health and Risk Management 4(3): 615-627). Development of the patch graft has reduced the incidence of adverse sequelae but is almost certainly not competitive for the purpose with the bypass described herein.

By slightly elevating the mean arterial pressure over the affected segment by elevating collateral cerebral blood flow through the middle cerebral artery (and/or reducing the cerebral metabolic rate through the use of general anesthesia which can be avoided, the risk of mid or postprocedural ischemic stroke or postoperative cognitive dysfunction as the result of endarterectomy is reduced, while healing after the procedure is expedited when the blood pressure across the affected segment is slightly reduced (see, for example, Heyer, E. J., Mergeche, J. L., Anastasian, Z. H., Kim, M., Mallon, K. A., and Connolly, E. S. 2014. “Arterial Blood Pressure Management During Carotid Endarterectomy and Early Cognitive Dysfunction,” Neurosurgery 74(3): 245-253; Mergeche, J. L., Bruce, S. S., Sander Connolly, E., and Heyer, E. J. 2014. “Reduced Middle Cerebral Artery Velocity During Cross-clamp Predicts Cognitive Dysfunction after Carotid Endarterectomy,” Journal of Clinical Neuroscience 21(3): 406-411; Wilson D. A., Mocco, J., D'Ambrosio, A. L., Komotar, R. J., Zurica, J., and 6 others 2008. “Post-carotid Endarterectomy Neurocognitive Decline is Associated with Cerebral Blood Flow Asymmetry on Post-operative Magnetic Resonance Perfusion Brain Scans,” Neurological Research 30(3): 302-306)(see also Domenick Sridharan, N., Chaer, R. A., Thirumal, P. D., Balzer, J., Long. B., Tzeng. E., Makaroun, M. S., and Avgerinos, E. D. 2017. “Somatosensory Evoked Potentials and Electroencephalography during Carotid Endarterectomy Predict Late Stroke but not Death,” Annals of Vascular Surgery 38:105-112; Jaffer, U., Normahani, P., Harrop-Griffiths, W., and Standfield, N. J. 2015. “Pre-operative Methods to Predict Need for Shunting During Carotid Endarterectomy,” International Journal of Surgery 23 (Part A): 5-11; Sussman, E. S., Kellner, C. P., Mergeche, J. L., Bruce, S. S., McDowell, M. M., Heyer, E. J., and Connolly, E. S. 2014. “Radiographic Absence of the Posterior Communicating Arteries and the Prediction of Cognitive Dysfunction after Carotid Endarterectomy,” Journal of Neurosurgery 121(3): 593-598; Pennekamp, C. W., van Laar, P. J., Hendrikse, J., den Ruijter, H. M., Bots. M. L., and 6 others 2013. “Incompleteness of the Circle of Willis is Related to EEG-based Shunting during Carotid Endarterectomy,” European Journal of Vascular and Endovascular Surgery 46(6): 631-637).

Complications associated with conventional carotid endarterectomy are both:

Midprocedural, to include intraoperative diagnostic indicators of probable sequelae (see, for example, Domenick Sridharan, N., Thirumala, P., Chaer, R., Balzer, J., Long. B., and 3 others 2018. “Predictors of Cross-clamp-induced Intraoperative Monitoring Changes during Carotid Endarterectomy Using Both Electroencephalography and Somatosensory Evoked Potentials,” Journal of Vascular Surgery (New York, New York) 67(1): 191-198; Norton, J. A., Peeling, L., Meguro, K., and Kelly, M. 2018. “Phenomenology of Neurophysiologie Changes during Surgical Treatment of Carotid Stenosis Using Signal Analysis,” Clinical Neurophysiology Practice 3:28-32; Reddy, R. P., Brahme, I. S., Karnati. T., Balzer, J. R., Crammond, D. J., Anetakis, K. M., and Thirumala, P. D. 2018. “Diagnostic Value of Somatosensory Evoked Potential Changes during Carotid Endarterectomy for 30-day Perioperative Stroke,” Clinical Neurophysiology 129(9): 1819-1831; Nwachuku, E. L., Balzer, J. R., Yabes, J. G., Habeych, M. E., Crammond, D. J., and Thirumala, P. D. 2015. “Diagnostic Value of Somatosensory Evoked Potential Changes during Carotid Endarterectomy: A Systematic Review and Meta-analysis,” Journal of the American Medical Association Neurology 72(1): 73-80; Inoue. T., Tsutsumi, K., Ohwaki, K., Tamura, A., Ono, H., Saito, I., and Saito, N. 2013. “Stratification of Intraoperative Ischemic Impact by Somatosensory Evoked Potential Monitoring, Diffusion-weighted Imaging and Magnetic Resonance Angiography in Carotid Endarterectomy with Routine Shunt Use,” Acta Neurochirurgica (Vienna, Austria) 155(11): 2085-2096; Mommertz, G., Das. M., Langer, S., Koeppel. T. A., Krings. T., and 3 others 2010. “Early Control of Distal Internal Carotid Artery during Carotid Endarterectomy: Does It Reduce Cerebral Microemboli?,” Journal of Cardiovascular Surgery (Turin, Italy) 51(3): 369-375; Ishigaki, D., Ogasawara, K., Suga, Y., Saito, H., Chida, K., and 4 others 2008. “Concentration of Matrix Metalloproteinase-9 in the Jugular Bulb during Carotid Endarterectomy Correlates with Severity of Intraoperative Cerebral Ischemia,” Cerebrovascular Disease 25(6): 587-592; Saito, H., Ogasawara, K., Komoribayashi, N., Kobayashi, M., Inoue. T., Otawara. Y., and Ogawa, A. 2007. “Concentration of Malondialdehyde-modified Low-density Lipoprotein in the Jugular Bulb during Carotid Endarterectomy Correlates with Development of Postoperative Cognitive Impairment,” Neurosurgery 60(6): 1067-1074; Pratesi, C., Dorigo, W., Innocenti, A. A., Azas, L., Barbanti. E., and 3 others 2004. “Reducing the Risk of Intraoperative Neurological Complications during Carotid Endarterectomy with Early Distal Control of the Internal Carotid Artery,” European Journal of Vascular and Endovascular Surgery 28(6): 670-673) and postprocedural (see, for example, AbuRahma, A. F., AbuRahma, Z. T., Scott, G., Adams, E., Mata, A., and 3 others 2019. “The Incidence of Carotid In-stent Stenosis is Underestimated >50% or >80% and Its Clinical Implications,” Journal of Vascular Surgery (New York, New York) 69(6): 1807-1814; Feng. Y., Li, L., Bai, X., Wang. T., Chen, Y., and 3 others 2019. “Risk Factors for New Ischaemic Cerebral Lesions after Carotid Artery Stenting: Protocol for a Systematic Review and Meta-analysis,” Online, British Medical Journal Open 9(8): e030025; Jeong, M. J., Kwon, S. U., Kim, M. J., Han, Y., Kwon, T. W., and Cho, Y. P. 2019. “Effects of Patient Age on Outcomes after Carotid Endarterectomy: A Retrospective, Single-center Study in Korea,” Medicine (Baltimore, Maryland) 98(32): e16781; Okazaki, T., Kanematsu, Y., Shimada, K., Korai, M., Satomi, J., and 3 others 2019. “A Single-center Retrospective Study with 5- and 10-year Follow-up of Carotid Endarterectomy with Patch Graft,” Neurologia Medico-chirurgica (Tokyo, Japan) 59(6): 231-237; Rizwan, M., Aridi, H. D., Dang. T., Alshwaily, W., Nejim. B., and Malas, M. B. 2019. “Long-term Outcomes of Carotid Endarterectomy and Carotid Artery Stenting when Performed by a Single Vascular Surgeon,” Vascular and Endovascular Surgery 53(3): 216-223; Rots. M. L., Meershoek, A. J. A., Bonati, L. H., den Ruijter, H. M., and de Borst, G. J. 2019. “Editor's Choice-Predictors of New Ischaemic Brain Lesions on Diffusion Weighted Imaging after Carotid Stenting and Endarterectomy: A Systematic Review,” European Journal of Vascular and Endovascular Surgery 58(2): 163-174; Traenka, C., Engelter, S. T., Brown, M. M., Dobson, J., Frost. C., and Bonati, L. H. 2019. “Silent Brain Infarcts on Diffusion-weighted Imaging after Carotid Revascularisation: A Surrogate Outcome Measure for Procedural Stroke? A Systematic Review and Meta-analysis,” European Stroke Journal 4(2): 127-143; Xin. W. Q., Li, M. Q., Li, K., Li, Q. F., Zhao. Y., and 4 others 2019. “Systematic and Comprehensive Comparison of Incidence of Restenosis between Carotid Endarterectomy and Carotid Artery Stenting in Patients with Atherosclerotic Carotid Stenosis,” World Neurosurgery 125:74-86; Sridharan, N. D., Chaer, R. A., Wu, B. B., Eslami, M. H., Makaroun, M. S., and Avgerinos. E. D. 2018. “An Accumulated Deficits Model Predicts Perioperative and Long-term Adverse Events after Carotid Endarterectomy,” Annals of Vascular Surgery 2018 46:97-103; Kumar, R., Batchelder, A., Saratzis, A., AbuRahma, A. F., Ringleb, P., and 4 others 2017. “Restenosis after Carotid Interventions and Its Relationship with Recurrent Ipsilateral Stroke: A Systematic Review and Meta-analysis,” European Journal of Vascular and Endovascular Surgery 53(6): 766-775; Orrapin, S. and Rerkasem, K. 2017. “Carotid Endarterectomy for Symptomatic Carotid Stenosis,” Cochrane Database of Systematic Reviews 6: CD001081; Garzon-Muvdi. T., Yang, W., Rong. X., Caplan, J. M., Ye, X., and 4 others 2016. “Restenosis after Carotid Endarterectomy: Insight into Risk Factors and Modification of Postoperative Management,” World Neurosurgery 89:159-167; Avgerinos, E. D., Go. C., Ling, J., Makaroun, M. S., and Chaer, R. A. 2015. “Survival and Long-term Cardiovascular Outcomes after Carotid Endarterectomy in Patients with chronic Renal Insufficiency,” Annals of Vascular Surgery 29(1): 15-21; Lye. C. R., Morrow, I. M., and Downs, A. R. 1982. “Carotid Artery Spasm. A Cause of Postendarterectomy Thrombosis?,” Archives of Surgery 117(12): 1531-1536).

Not only is the incidence of post carotid open endarterectomy myocardial infarction lower than when treated by angioplasty and stenting, but assuming the reason is spasm of the coronary arteries, use of the means described herein should drive the incidence even lower if preceded by treatment of the coronaries (see, for example, Hye, R. J., Voeks, J. H., Malas, M. B., Tom, M., Longson, S., Blackshear, J. L., and Brott. T. G. 2016. “Anesthetic Type and Risk of Myocardial Infarction after Carotid Endarterectomy in the Carotid Revascularization Endarterectomy versus Stenting Trial (CREST),” Journal of Vascular Surgery (New York, New York) 64(1): 3-8; Boulanger, M., Cameliere. L., Felgueiras. R., Berger, L., Rerkasem, K., Rothwell, P. M., and Touze, E. 2015. “Periprocedural Myocardial Infarction after Carotid Endarterectomy and Stenting: Systematic Review and Meta-Analysis,” Stroke 46(10): 2843-2848; Blackshear, J. L., Cutlip, D. E., Roubin, G. S., Hill. M. D., Leimgruber, P. P. and 8 others 2011. “Myocardial Infarction after Carotid Stenting and Endarterectomy: Results from the Carotid Revascularization Endarterectomy Versus Stenting Trial,” Circulation 123(22): 2571-2578).

The endarterectomy open, if vulnerable, plaque in the coronaries is best eliminated or the coronaries replaced by bypass grafts or by using the means and methods described in U.S. Pat. No. 11,389,171, entitled Integrated System for the Infixion and Retrieval of Implants. If the condition of the coronaries is not considered to pose a low threat of mid or postprocedural myocardial infarction, an antispasmodic such as phenoxybenzamine hydrochloride topical, or less desirably, oral (10 milligrams capsules) is administered.

If the coronaries are treated and left, these as well as the common carotid or carotids are jacketed by a ductus side-entry jacket as shown in U.S. Pat. No. 11,759,186 with surface port as shown herein to deliver the antispasmodic such as phenoxybenzamine hydrochloride as necessary by direct targeting, thus eliminating its adverse side effects such as tachycardia (see also Rinkinen, J. and Halvorson, E. G. 2017. “Topical Vasodilators in Microsurgery: What Is the Evidence?,” Journal of Reconstructive Microsurgery 33(1): 1-7; Choi, S., Kwon, H. J., Song. H. J., Choi, S. W., Nagar, H., and 5 others 2016. “Nafamostat Mesilate Promotes Endothelium-dependent Vasorelaxation via the Akt [serine/threonine kinase B]-eNOS [endothelial nitric oxide synthase] Dependent Pathway,” Korean Journal of Physiology and Pharmacology 20(5): 539-545; Vargas, C. R. Iorio, M. L., and Lee. B. T. 2015. “A Systematic Review of Topical Vasodilators for the Treatment of Intraoperative Vasospasm in Reconstructive Microsurgery,” Plastic and Reconstructive Surgery 136(2): 411-422; Yu, J. T., Hwang, S., Lee, H.-J., Kim, G., Won, K.-J., Par, Y. S., and Jo, I. 2015. “CCN1 Acutely Increases Nitric Oxide Production via Integrin αvβ3-Akt-S6K-phosphorylation of Endothelial Nitric Oxide Synthase at the Serine 1177 Signaling Axis,” Free Radical Biology and Medicine 89:229-240; Kang. M W, Song. H. J., Kang, S. K., Kim, Y., Jung, S. B., and 6 others 2015. “Nafamostat Mesilate Inhibits TNF [tumor necrosis factor]-α-Induced Vascular Endothelial Cell Dysfunction by Inhibiting Reactive Oxygen Species Production,” Korean Journal of Physiology and Pharmacology 19(3): 229-234; Yoon, Y. S., Oh, H., Kim, Y., Lim, S. P., Kim, C. S., and Kang, M. W. 2013. “Nafamostat Mesilate: Can It be Used as a Conduit Preserving Agent in Coronary Artery Bypass Surgery?,” Korean Journal of Thoracic and Cardiovascular Surgery 46(6): 413-425; Patel, A. J., and Malata, C. M. 2011. “The Use of Topical Vasodilators in Microvascular Surgery,” Journal of Plastic, Reconstructive, and Aesthetic Surgery 64(2): 226-228; Loh, C. Y., Lim, P. C., and Lam, D. G. 2011. “Re: The Use of Topical Vasodilators in Microvascular Surgery,” Journal of Plastic, Reconstructive, and Aesthetic Surgery 64(12): 1726; Mussa. S., Guzik, T. J., Black, E., Dipp, M. A., Channon, K. M., and Taggart. D. P. 2003. “Comparative Efficacies and Durations of Action of Phenoxybenzamine, Verapamil/Nitroglycerin Solution, and Papaverine as Topical Antispasmodics for Radial Artery Coronary Bypass Grafting,” Journal of Thoracic and Cardiovascular Surgery 126(6): 1798-1805).

To bypass all flow through the common carotid and diverge this stream in the proper proportion between the ipsilateral internal and external carotids, a nonadjustable or prosthesis-type jacket as shown in FIGS. 2 and 3 is positioned along the common carotid inferior to the proposed treatment site. If the plaque is highly calcified, the jacket is best situated just outside this area. The surgery open, once the jacket is placed, the operator controls chute deployment manually, without the need for body surface port knob or radio remote control as is necessary with full implantation. An accessory channel of the jacket conveys the blood to a stream-splitting (divergence, fork) Y-joint, which is a mid-diversion line or off-line surrogate heterotopic or ectopic synthetic replica of a native carotid bifurcation.

Eliminating protracted occlusion or clamping, almost certainly the cause of mid or postprocedural ischemic stroke, and significant changes in blood pressure, eliminates much of the complications and adverse sequelae associated with an open endarterectomy (see, for example, Morris. D. R., Ayabe, K., Inoue. T., Sakai, N., Bulbulia. R., Halliday, A., and Goto, S. 2017. “Evidence-Based Carotid Interventions for Stroke Prevention: State-of-the-art Review,” Journal of Atherosclerosis and Thrombosis 24(4): 373-387; Featherstone, R. L., Dobson, J., Ederle, J., Doig. D., Bonati, L. H., Morris, S. Patel, N. V., and Brown, M. M. 2016. “Carotid Artery Stenting Compared with Endarterectomy in Patients with Symptomatic Carotid Stenosis (International Carotid Stenting Study): A Randomised Controlled Trial with Cost-effectiveness Analysis,” Health Technology Assessment 20(20): 1-94; Paraskevas, K. I., Kalmykov, E. L., and Naylor, A. R. 2016. “Stroke/Death Rates Following Carotid Artery Stenting and Carotid Endarterectomy in Contemporary Administrative Dataset Registries: A Systematic Review,” European Journal of Vascular and Endovascular Surgery 51(1): 3-12; Joviliano, E. E., Yoshida, W. B., Sobreira, M. L., Moura, R., Guillaumon, A. T., and 4 others 2016. “An Observational Registry of Carotid Endarterectomy and Carotid Artery Stenting in Brazil: Study Protocol,” JMIR [Journal of Medical Internet Research], Research Protocols 5(4): e226; Bonati, L. H., Dobson, J., Featherstone, R. L., Ederle, J., van der Worp. H. B., with 9 others and 298 collaborators 2015. “Long-term Outcomes after Stenting Versus Endarterectomy for Treatment of Symptomatic Carotid Stenosis: The International Carotid Stenting Study (ICSS) Randomised Trial,” Lancet (London, England) 385(9967): 529-538; Nanba. T., Ogasawara, K., Nishimoto, H., Fujiwara, S., Kuroda, H., Sasaki, M., Kudo, K., and 4 others 2012. “Postoperative Cerebral White Matter Damage Associated with Cerebral Hyperperfusion and Cognitive Impairment after Carotid Endarterectomy: A Diffusion Tensor Magnetic Resonance Imaging Study,” Cerebrovascular Diseases (Basel) 34(5-6): 358-367). The bypass return jackets are positioned along the internal and external carotids above the level of the treatment segment.

Based upon patient age, race, sex, and medical condition, the absolute dimensions of the common carotid jacket accessory channel carrying the full flow, the lines within the stream-splitting Y-joint, and the lines connecting these to the receiving side-entry jackets along the internal and external carotids are subject to variation. Best results are obtained when the dimensions of the prosthetic bypass match those of the native vessels (see, for example, Rashid, S. A. and Mahmud, S. A. 2015. “Correlation between Carotid Artery Intima-Media Thickness and Luminal Diameter with Body Mass Index and Other Cardiovascular Risk Factors in Adults,” Sultan Qaboos University Medical Journal 15(3): e344-e350; Vaidya, D., Bennett, W. L., Sibley, C. T., Polak, J. F., Herrington, D. M., and Ouyang, P. 2014. “Association of Parity with Carotid Diameter and Distensibility: Multi-ethnic Study of Atherosclerosis,” Hypertension 64(2): 253-258; Markert. M. S., Della-Morte, D., Cabral. D., Roberts, E. L. Jr, Gardener, H., and 5 others 2011. “Ethnic Differences in Carotid Artery Diameter and Stiffness: The Northern Manhattan Study,” Atherosclerosis 219(2): 827-832).

The luminal diameter of the common carotid varies considerably, from 4.3 to 7.7 millimeters, with the internal and external carotids varying between 5.78+/−0.57 and 5.86+/−0.66 millimeters in the indigenous people of Nepal with an inconsequentially small increase in diameter among young adult men (Limbu, Y. R., Gurung, G., Malla. R., Rajbhandari. R., and Regmi, S. R. 2006. “Assessment of Carotid Artery Dimensions by Ultrasound in Non-smoker Healthy Adults of Both Sexes,” Nepal Medical College Journal 8(3): 200-203); (see also Arat. Y. O., Arat. A., and Aydin, K. 2015. “Angiographic Morphometry of Internal Carotid Artery Circulation in Turkish Children,” Turkish Neurosurgery 25(4): 608-616; Dillon, W. P., Barkovich, A. J., Norman, D., Dowd, C. F., and Kane. T. T. 1996. “Reduced Caliber of the Internal Carotid Artery: A Normal finding with Ipsilateral Absence or hypoplasia of the A1 segment,” American Journal of Neuroradiology 17(7): 1295-1301).

In instances of extracranial carotid agenesis (aplasia) or hypoplasia, or where intracranial extension of the defect still allows connection, use of the device described as a permanent implant prosthesis can be used to improve circulation to the brain (see, for example, Li, S., Hooda, K., Gupta, N., and Kumar, Y. 2017. “Internal Carotid Artery Agenesis: A Case Report and Review of Literature,” Neuroradiology Journal 30(2): 186-191; Wali, A. R., Santiago-Dieppa, D. R., Steinberg, J. A., Alattar, A., Cheung, V. J., Modir, R., Khalessi, A. A., and Pannell, J. S. 2016. “Hypoplastic Internal Carotid Artery Co-presenting with Neurofibromatosis and Intracranial Masses,” Online, Cureus (Palo Alto, California) 8(8): e750; Khan, A., Baheerathan, A., Hussain, N., and Gosalakkal, J. 2012. “Agenesis of the Internal Carotid Artery Associated with Ipsilateral Horner Syndrome in a Child,” Pediatric Neurology 47(1): 68-70; Kiritsi, O., Noussios, G., Tsitas, K., and Lappas, D. 2012. “Unilateral agenesis of the Internal Carotid Artery Presented as Transient Ischaemic Attack: A Case Report,” Surgical and Radiologic Anatomy (Berline, Germany) 34(5): 475-477; Zink, W. E., Komotar, R. J., and Meyers, P. M. 2007. “Internal Carotid Aplasia/Hypoplasia and Intracranial Saccular Aneurysms: Series of Three New Cases and Systematic Review of the Literature,” Journal of Neuroimaging 17(2): 141-147; Kane, A. G., Lee, J. H., Oh, C. W., Lee, S. H., and Han, D. H. 2003. “Aplasia of the Internal Carotid Artery,” Acta Neurochirurgica (Vienna, Austria) 145(2): 117-125).

To preserve laminar, or streamline flow, the passageway through the joint curves gradually and the internal walls are seamless and smooth throughout. The tubing used is nonthrombogenic, such as plasticizer-free polyvinyl chloride (see, for example, Posnack, N. G. 2014. “The Adverse Cardiac Effects of Di(2-ethylhexyl)phthalate and Bisphenol A,” Cardiovascular Toxicology 14(4): 339-357; Genay, S., Luciani, C., Decaudin, B., Kambia, N., Dine, T., and 4 others 2011. “Experimental Study on Infusion Devices Containing Polyvinyl Chloride: To What Extent are They Di(2-ethylhexyl)phthalate-free?,” International Journal of Pharmaceutics 412(1-2): 47-51; Hill, S. S., Shaw, B. R., and Wu, A. H. 2001. “The Clinical Effects of Plasticizers, Antioxidants, and Other Contaminants in Medical Polyvinylchloride Tubing during Respiratory and Non-respiratory Exposure,” Clinica Chimica Acta 304(1-2): 1-8), and if necessary, a suitable anticoagulant, such as low molecular weight heparin is delivered through the accessory channel of the common carotid jacket.

Rather than cutting off blood flow through the carotids during incision—with some transient disruption in blood pressure, loss of blood, and probable ipsilateral hemisphere hypoxia—and extraction of the atheroma, preparation and stitching a patch in place if necessary, and stitching the incision closed, thus entrusting perfusion and postprocedural clear-headedness to collateral circulation as in the conventional procedure (see, for example, Messina, L. M. and Zelenock, G. B. 1997. “Cerebrovascular Occlusive Diseasel ,” Chapter 80 in Greenfield, L. J., Mulholland, M., Oldham, K. T., Zelenock, G. B., and Lillemoe, K. D., (eds.) Surgery: Scientific Principles and Practice, Philadelphia, Pennsylvania: Lippincott Raven, pages 1753-1757), retraction of the jacket diversion chute instantly switches flow from the native common carotid to the bypass device with no more than a slight transient effect upon the blood pressure.

Fully extending the diversion chute into the common carotid thus suddenly shifts continued flow to the branches and brain, diverting all flow into the bypass leading to a side-entry jackets positioned superior to the proposed treatment site on the internal and external carotid. By eliminating the discontinuities in arterial pressure of the conventional procedure, the carotid endarterectomy described below considerably reduces if not eliminates the risk of mid or postprocedural stroke as well as other complications. Instant flow switching minimizes hypoxia, and the direct targeting of an anticoagulant or platelet blockade to the treatment site without exposure of the rest of the body much reduces and probably eliminates the risk of thrombogenic ischemia.

Provided the jackets and the common mainline connecting these have been properly placed, the usual one to five percent of patients who experience significant hemorrhage (Messina, L. M. and Zelenock, G. B. 1997, Op cit., page1755-1756). If the patient has shown a propensity to symptomatically restenose due to intimal hyperplasia or recurrent atherosclerosis following a first carotid endarterectomy, the device is left in place as a prosthesis. Otherwise, in a first carotid endarterectomy, unless there is a continued need for the device to correct a malformity or to replace damaged tissue, it is removed.

When, as arises frequently, the endarterectomy or less frequently, aneurysmal repair, is at the bifurcation, or bulb, of the carotid artery, the regularity, and if appropriate, precise localized mechanical or medicinal control over the blood pressure protects the patient both during and after the operation. Using a bypass of the kind described, unless intentionally manipulated by adjusting any of the chutes or through the use of drugs, any change in blood pressure is very brief, when the operator switched-organes flow from the common carotid into the bypass, and when the device is removed returning flow to the internal and external carotids.

Where the vasculature has been irreparably damaged as the result of trauma, the device is left in place as a prosthesis with an accessory channel line to a surface port for anticoagulant drip replenishment. However, midprocedural use and placement as a prosthesis represent extremes: when the vessels are impaired but not lost, these can be anastomosed. Whether to alleviate the healing native vessels from continuous shear stress or avert an impending rupture, for example, the device can then be triggered by implanted sensors to automatically switch from the prosthesis to the native vessels or the reverse.

Blood flow continuous throughout the procedures, the hemispheres experience little if any change in mean arterial pressure, significant cerebral blood flow asymmetry, reperfusion, or hyperperfusion syndrome, so that unless the patient is an infant or demented and agitated, the procedure is performed under local anesthesia. This combined with flow and pressure continuity further dispel concerns that the patient will experience postprocedural cognitive impairment (Wang, T., Wang, X., He, Y., Zhang, T., Song, J., Bai, X., and Han, C. 2015. “Decreased ¹³N-labeled Ammonia Uptake in the Ipsilateral and Contralateral Hemispheres following Carotid Endarterectomy,” Molecular Medicine Reports 12(5): 6598-6604; Feinkohl, 1., Winterer, G., Spies, C. D., and Pischon, T. 2017. Cognitive Reserve and the Risk of Postoperative Cognitive Dysfunction,” Deutsches Arzteblatt International 114(7): 110-117; Farooq, M. U., Goshgarian, C., Min, J., and Gorelick, P. B. 2016. “Pathophysiology and Management of Reperfusion Injury and Hyperperfusion Syndrome after Carotid Endarterectomy and Carotid Artery Stenting,” Experimental and Translational Stroke Medicine 8(1): 7; Oshida, S., Ogasawara, K., Saura, H., Yoshida, K., Fujiwara, S., and 5 others 2015. “Does Preoperative Measurement of Cerebral Blood Flow with Acetazolamide Challenge in Addition to Preoperative Measurement of Cerebral Blood Flow at the Resting State Increase the Predictive Accuracy of Development of Cerebral Hyperperfusion after Carotid Endarterectomy? Results from 500 Cases with Brain Perfusion Single-photon Emission Computed Tomography Study,” Neurologia Medico-Chirurgica (Tokyo, Japan) 55(2): 141-148; Uchida, K., Yoshimura, S., Shirakawa, M., Shindo, S., Egashira, Y., Iwamna, T., and Yamada, K. 2015. “Experience of Staged Angioplasty to Avoid Hyperperfusion Syndrome for Carotid Artery Stenosis,” Neurologia Medico-Chirurgica (Tokyo, Japan) 55(11): 824-829; Yoshida, K., Ogasawara, K., Saura, H., Saito, H., Kobayashi, M., and 4 others 2015. “Post-carotid Endarterectomy Changes in Cerebral Glucose Metabolism on (18)F-fluorodeoxyglucose Positron Emission Tomography Associated with Postoperative Improvement or Impairment in Cognitive Function,” Journal of Neurosurgery 123(6): 1546-1554; Rundshagen, I. 2014. “Postoperative Cognitive Dysfunction,” Deutsches Arzteblatt International 111(8): 119-125; Evered, L., Scott, D. A., Silbert, B., and Maruff, P. 2011. “Postoperative Cognitive Dysfunction is Independent of Type of Surgery and Anesthetic,” Anesthesia and Analgesia 112(5): 1179-1185; Sauer, A. M., Kalkman, C., and van Dijk, D. 2009. Postoperative Cognitive Decline,” Journal of Anesthesia 23(2): 256-259; Wilson D. A., Mocco, J., D'Ambrosio, A. L., Komotar, R. J., Zurica, J., and 6 others 2008, Op cit.; Mocco, J., Wilson, D. A., Komotar, R. J., Zurica, J., Mack, W. J., and 5 others 2006. “Predictors of Neurocognitive Decline after Carotid Endarterectomy,” Neurosurgery 58(5): 844-850).

Turning now to FIGS. 25A and 25B, before initiating a carotid endarterectomy, a blood flow diversion jacket with at least two accessory channels is positioned along the common carotid at a level below or inferior to the proposed endarterectomy, with the jacket inverted so that blood rising up through the common carotid is diverted craniad (cephalad, upwards). One accessory channel is dedicated as a bypass catheter to a surrogate carotid bifurcation prosthesis, thence to side-entry jackets along the internal and external carotids at levels above the proposed treatment site. With the chute fully retracted, blood flows unimpeded and without damage up into the internal and external carotids. The chute fully extended, all flow through the common carotid is diverted through the jacket mainline to bypass the treatment site with little if any alteration in flow up into the head.

In an open endarterectomy using the method and apparatus described herein, all flow through the common carotid is diverted midprocedurally and not diverted at all postprocedurally. Diverted or not, one or more accessory channels of the jacket on the common carotid can be used to directly target drugs into the stream flowing craniad (cephalad, upwards) through it, infusion at that level delivered to both branches. And diverted or not, the accessory channels of the jackets on the internal and external branches cranial to or above the defect can likewise be used to directly infuse medication, but differently to each branch and the supply territory of each branch. For example, an accessory channel of the jacket on the internal carotid can target drugs to the brain without medicating the tongue, face, or thyroid gland, which are supplied by the external carotid.

The creation of a bypass aside, side-entry jackets and side-entry diversion jackets allow direct infusion into any vessel or its branches that can be jacketed. Blood pressure increase through the internal carotid to avert cognitive impairment is by the release of topically active vasoconstrictors such as phenoxybenzamine, oxymetazoline hydrochloride, or desglymidodrine, additional such agents specified above in section 2a, entitled Background of the Invention. General, through an accessory channel of the side-entry jacket on the internal carotid, the absolute volume of the drug far too small to induce a reflex tachycardia.

Here where diversion closely approximates, essentially duplicates, normal flow, the need to variously apportion flow rather than drugs between the branches does not apply. However, in other contexts, side-entry diversion jackets would allow any relative portion of blood or urine passing through the valve jacket to be diverted. In open surgery, the adjustment is performed manually by the operator, whereas when fully implanted, adjustment is by telemetric signaling to the clinic and remote radio control, or by intracorporeal control by the implanted microcontroller.

During diverted flow, drugs topically active in the brainstem, or more specifically, the locus ceruleus, to afford a centrally mediated sympatholytic effect to lower the mean systemic and pulmonary arterial pressures without the need for angiotensin converting enzyme inhibition, which produces a postprocedural rebound increase in the systemic blood pressure and added protection against postprocedural cognitive impairment such as dexmedetomidine (Ge, Y. L., Li, X., Gao, J. U., Zhang, X., Fang, X., Zhou, L., Ji, W., and Lin, S. 2016. “Beneficial Effects of Intravenous Dexmedetomidine on Cognitive Function and Cerebral Injury Following a Carotid Endarterectomy,” Experimental and Therapeutic Medicine 11(3): 1128-1134; Zhang, H. P., Ma, X. D., Chen, L. F., Yang, Y., Xu, B. N., and Zhou, D. B. 2016. “Cognitive Function after Carotid Endarterectomy: Early Decline and Later Recovery,” Turkish Neurosurgery26(6): 833-839; Nair, A. S. 2014. “Benefits of Using Dexmedetomidine during Carotid Endarterectomy: A review,” Saudi Journal of Anaesthesia 8(2): 264-267) is released either by the side-entry diversion jacket in the doublet assigned to the internal carotid through its accessory channel into the return jacket on the internal carotid or directly by the jacket on the internal carotid.

2b(2)(h). Local Vascular Pressure Relief

Relief in local hypertension such as portal or pulmonary by means of tapping off a volume of blood for delivery into a large vessel of the same kind is a palliative, not a curative measure, intended to protect downstream tissue from injury until the underlying medical problem is alleviated. The medical object is to alleviate the cause, usually a downstream cause of obstruction. Different devices for the relief of a localized elevation in blood pressure are addressed, bidirectional servovalves having already been addressed above in section 2b(2)(b), entitled Bidirectional Valves for Perinatal Extracardiac Correction of Complete or Dextro-transposition of the Great Arteries and Reciprocal Cross-circulation, with valved shunts and the applications for these covered below in sections 2b(2)(h)(h1), entitled The ‘Tuned’ Shunt, thru section 2b(2)(h)(h4), entitled The ‘Tuned’ Shunt in the Relief of Localized Arterial Hypertension.

The different devices are negative feedback-controlled actuators in closed loop-driven servomechanisms responsive to sensor feedback. The distinctions between anastomosed bypasses and tuned shunts as addressed in the section to follow, 2b(2)(h)(h1), entitled The ‘Tuned’ Shunt, is that the tuned shunt is continuously variable in adjusting the pressure responsive to the immediate activity level of the patient and is equipped with at least one accessory channel which can accept a drugline to release medication of any kind into the connectors and shunt. In this regard, a shunt made using the expandable structure delineated above in section 2b(2)(f), entitled Expandable Structure to Accommodate Growth and Self-adjust in Size in Vascular Segment Prostheses, Lines, and Confluence Conduits Placed in Children, has an inherent ability to spontaneously adjust in dimension responsive to the instant blood pressure.

High blood pressure can precipitate an arterial or venous aneurysm alone or in combination with structural weakening of the vessel by infection, a connective tissue disorder, or a vasculitis, for example. An aneurysmal, pseudoaneurysmal, dissected, fistulized, or otherwise damaged or diseased-such as a severely atherosclerosed or healably infected segment along a vessel—can be simultaneously and automatically excised and replaced by an inline coupling jacket bridge insert. Elevated pressure is quantified by sensors, usually thin-film within, or girthing sphincteric-type encircling, the affected vessel. The term ‘tuned’ is intended to denote that the servovalve used to continuously or intermittently adjust the partial diversion of blood from the hypertensive vessel to relieve the excessive pressure is controlled on the basis of sensor data and negative feedback.

Alternatively, where the native vessel is anticipated to heal spontaneously or following an intervention, a segment bypass line connected at the upstream and downstream ends by side-entry solenoid-driven valves allows periodic switching between the native and bypass lines, and using servovalves allows the apportionment of the total volume of flow between the native and synthetic bypass lines. One or more accessory channels in inline coupling jackets allow the directly delivery into the synthetic replacement line or maintenance agents such as anti-thrombotic in the vascular tree or crystal dissolving in the urinary tract.

Bypass line jackets can incorporate accessory channels that allow the direct delivery into the native segment of medication such as an anti-inflammatory and into the connectors of maintenance agents such as anti-thrombotic in the vascular tree or crystal dissolving in the urinary tract. Valving to switch between the native and synthetic lines is automatically coordinated with the delivery of medicinal or maintenance agents by a totally implanted microcontroller-administered system- or in the case of comorbid disease—by a totally implanted control microprocessor-administered system executing a hierarchical prescription-program.

This coordination can additionally be supplemented automatically with control of electromechanical means for locally adjusting the blood pressure other than medicinal such as directly targeted nitroglycerin. Included are servochokes, electromagnetic sphincters, and nonchoke-type blood pressure relief servovalves that tap off a volume of flow and pass it into a nearby large vessel. By the same token, if the pressure is too low, some flow can be diverted from a large nearby artery. To control high blood pressure that fluctuates, these can be connected directly to the native artery downstream to the exit coupling valve-jacket or along the synthetic bridge-mounted vessel replacement segment or both.

Where the pressure is too high but constant, the tap need consist of no more than a basic ductus side-entry diversion jacket connected directly to the native artery or connected indirectly along the synthetic replacement bridge with a catheter to pass the blood drawn off into a larger vessel nearby. Means for dispelling local hypertension such as pulmonary, portal, and renal are addressed in sections to follow, to include section (e4). The ‘Tuned’ Shunt in the Relief of Localized Arterial Hypertension: The backup jacket or servovalve always incorporates at least one accessory channel to allow the direct pipeline targeting of blood pressure reducing medication to the site; however, mechanical reduction should resolve all but refractory cases of high blood pressure.

Directly applied somewhat short of the aortic root to tap off blood to other large arteries by apportioning it throughout the body, such a servovalve or servovalves should ameliorate hypertensive pressure that causes damage to the lungs. Related sections include 2b(2)(c)(c2), entitled Automatic Insertion into a Ductus of an Inline Coupling jacket-Secured Bridging, or Tie-line and Its Replacement if Necessary, 2b(2)(d), entitled Anoxia-free Aortic Repair: Aortic Prostheses Thoracic, Thoracoabdominal, and Abdominal, and 2b(2)(h)(h1), entitled The ‘Tuned’ Shunt, and its subsections. By such means, the relief of elevated blood pressure is accomplished as a causative or contributing factor to aneurysmal failure of the arterial wall.

Vascular servovalves applied thus are continuously and dynamically adjusted in the proportion of blood diverted to another large vessel by an implanted microcontroller or—in a comorbidity multiple channel hierarchical control system, microprocessor-executing a prescription-program to accommodate a heightened level of exertion and changes in posture (see, for example, Ishii, W., Fujita, Y., Kawaguchi, T., Kimura, K., Fukuda, A., Fuchigami, T., and Morioka, 1. 2020. “Cerebral Autoregulation during Active Standing Test in Juvenile Patients with Instantaneous Orthostatic Hypotension,” Advances in Experimental Medicine and Biology 1232:77-83. Mol, A., Maier, A. B., van Wezel, R. J. A., and Meskers, C. G. M. 2020. “Multimodal Monitoring of Cardiovascular Responses to Postural Changes,” Online, Frontiers in Physiology 11:168; Lazaro, J., Gil, E., Orini, M., Laguna, P., and Bailon, R. 2019. “Baroreflex Sensitivity Measured by Pulse Photoplethysmography,” Online, Frontiers in Neuroscience 13:339; Mol, A., Woltering, J. H. H., Colier, W. N. J. M., Maier, A. B., Meskers, C. G. M., and van Wezel, R. J. A. 2019. “Sensitivity and Reliability of Cerebral Oxygenation Responses to Postural Changes Measured with Near-infrared Spectroscopy,” European Journal of Applied Physiology 119(5): 1117-1125) and emotional state. Sensor function thus mimics and can report the functioning of the carotid and aortic arch bodies.

A local elevation in blood pressure with a reduction in blood pressure downstream to the causative obstruction is dealt with endoluminally (endovascularly, transluminally, perhaps transcatheterically), by stenting, or if due to tamponade, then by tying away the encroaching tissue to a retracted position. Severe blockages, such as a congenitally interrupted aortic arch are dealt with below in section 2b(2)(d), entitled Anoxia-free Aortic Repair: Aortic Prostheses Thoracic, Thoracoabdominal, and Abdominal. Less severe obstructions due to congenital malformities, kinks, and buckles can be dealt with or deferred for later repair expeditiously, less traumatically, and without the risk of later anastomosis dehiscence through the use of ductus side-entry jackets and shunts. However, the risk of thromboembolism posed by thrombus or vulnerable plaque must be cleared at once.

Lesions or clot that occlude or protrude into the lumen include deep vein thromboses (see, for example, Packer, C. F., Bickel, S., and Dattilo. J. B. 2020. “Vein Obstruction,” Treasure Island, Florida: StatPearls Publishing, Online at https://www.ncbi.nlm.nih.gov/books/NBK537255/, vessel tortuosity, buckling, or collapse (see, for example, Garcia, J. R., Sanyal, A., Fatemifar, F., Mottahedi, M., and Han, H.-C. 2017. “Twist Buckling of Veins under Torsional Loading,” Journal of Biomechanics 58:123-130; Fatemifar, F. and Han, H.-C. 2016. “Effect of Axial Stretch on Lumen Collapse of Arteries,” Journal of Biomechanical Engineering 138(12); Garcia, J. R., Lamm, S. D., and Han, H.-C. 2013. “Twist Buckling Behavior of Arteries,” Biomechanics and Modeling in Mechanobiology 12(5): 915-927; Han, H.-C., Chesnutt, J. K., Garcia, J. R., Liu, Q., and Wen, Q. 2013. “Artery Buckling: New Phenotypes, Models, and Applications,” Annals of Biomedical Engineering 41(7): 1399-1410; Liu, Q. and Han, H.-C. 2012. “Mechanical Buckling of Artery under Pulsatile Pressure,” Journal of Biomechanics 45(7): 1192-1198; Han, H.-C. 2012. “Twisted Blood Vessels: Symptoms, Etiology and Biomechanical Mechanisms,” Journal of Vascular Research (Basel, Switzerland) 49(3): 185-197; Martinez, R., Fierro, C. A., Shireman, P. K., and Han, H.-C. 2010. “Mechanical Buckling of Veins under Internal Pressure,” Annals of Biomedical Engineering 38(4): 1345-1353), encroachment by neighboring tissue (see, for example,) or atherosclerotic plaque, for example, demands removal of the cause.

An important consideration in system design is the avoidance of needless complexity. For example, following heart transplantation in an older patient unable or unlikely to engage in strenuous exercise, the denervated heart can be expected to perform at a substantially consistent level, making extraordinary measures to recover innervated performance gratuitous. The same applies to a younger patient who nonathletic, is amenable to limiting health-maintaining exercise to less stressful levels.

Advantages in a synthetic shunt as opposed to one created surgically are primarily that 1. Using servovalves, the flow-through rate can be automatically and instantly adjusted continuously in response to the activity level of the patient or another condition that increases demand, one or more pressure sensors along the internal wall of the shunt taken by the implanted microprocessor to adjust the degree of diversion chute extension, hence, the relative proportion of flow diverted, usually to a large neighboring vein, 2. Eliminating the need to harvest a vessel to create the shunt avoids numerous complications that might arise in a nonpediculed, hence, unperfused tissue and/or at the harvest site, and 3. The diversion servovalve at the inlet and if present, that at the outlet provide accessory channels, or druglines, that allow fouling due to the accumulation of clot, crystal, or biofilm to be prevented or removed without reentry.

Depending upon the specific condition of the patient, all three devices have potential application. In the abstract, however, a listing in increasing order of preference is:
1. The extravascular choke servovalve, or servochoke, an electrical sphincter, as affording dynamically adaptive banding superior to conventional adjustable banding.
2. The intravascular servochoke, which advances and retracts an obturator into the native lumen without constriction of the vessel and is therefore preferred to the extravascular choke.
3. The diversion valve, which taps off a portion of blood for transfer to a larger vessel or from a large vessel to one or more smaller vessels. Not shifting the hypertension upstream with possible adverse consequences, the diversion servovalve is preferred to either type of choke.

As described above in section 2b(1)(b)(b5), in double heart metered compound vascular bypass, or sudden switched-organ transplantation [and so on], two hearts, either or both of which exhibit reduced left ventricular ejection fraction, or absolute stroke volume, but when coupled together are able to generate adequate output volume through increased pulse rate, inotropes if necessary used to raise the pressure, are connected in a novel parallel manner whereby ejection and venous return are exchanged between the two, the relative proportion of output biased in favor of that heart more competent. Such a procedure is intended as a fallback or bailout strategy to be resorted to only when the condition of the patient is exigent, and a fully competent replacement heart is unavailable, a ventricular assist device is unavailable or the use thereof contraindicated, or an orthotopically replaced heart falls short of the performance that had been expected of it.

By allowing the use of less than ideal hearts, the procedure considerably increases the pool of hearts acceptable for transplantation. Following a double heart metered compound vascular bypass, or sudden switched-organ transplantation, two left and two right ventricles eject blood into the aorta and the main or right and left pulmonary arteries. If the two hearts generate the same output power, the vascular servovalve diversion chutes are set at the halfway point and toggled at the sensor-fed microcontroller algorithm-determined optimal empirical rate and extent for recovery around that combination of setpoints.

In this circumstance, normal blood pressure in these great vessels is maintained by the continuous detection of the blood pressure in each by the automatic diversion to smaller vessels of the volume of blood necessary to return the pressure to within the normal range. To accomplish this action, concentric circumferential strain gauge pressure sensors internal to the vascular servovalve-jackets immediately pass the abnormal value to the implanted system microcontroller. The microcontroller then and responded to by adjustment of vascular valves is automatically and continuously controlled by the implanted automatic disorder response system using these means, primarily that specified at 3.

Constrictive banding to reduce pressure downstream, beyond the band, is done to prevent injury to the organ supplied, typically the lungs or liver. Intravascular choke servovalves are not bands as such but accomplish the same result without constricting the substrate vessel. Conventional bands and extravascular choke servovalves are constrictive, but only the latter is spontaneously adaptive as an electrical sphincter. Extravascular and intravascular choke servovalves serve the same function; however, an intravascular choke is less deleterious. The effect of constantly constricting a vessel by the sphincteric device can eventually injure, and has been known even to erode the vascular adventitia, induce atheromatous degeneration in the intima.

However, having been used mostly as a bridging method to allow the infant to stabilize prior surgical repair, the immediate benefits outweighed any consequences which would not become evident before the bands would be removed. Banding is still functional on a temporary basis (see, for example, Durandy, Y. 2013. “Pulmonary Artery Banding is Still a Valuable Option,” Online, Frontiers in Pediatrics 2013 1:17). When compliance in appearing for surgical repair is lacking so that the adverse consequences of long-term use ensue, banding results in numerous needless deaths (see, for example, Brooks, A., Geldenhuys, A., Zuhlke, L., Human, P., and Zilla, P. 2012. “Pulmonary Artery Banding: Still a Valuable Option in Developing Countries?,” European Journal of Cardiothoracic Surgery 41(2): 272-276).

Use of even an adaptive artificial sphincter much less the other devices specified as preferred thereto as addressed herein, especially the use of a diversion valve, all with the potential to serve on a permanent basis would prevent these deaths. This makes the situation of a child with pulmonary hypertension following surgical repair safer (see, for example, Latus, H., Wagner, I., Ostermayer, S., Kerst, G., Kreuder, J., Schranz, D., and Apitz, C. 2017. “Hemodynamic Evaluation of Children with Persistent or Recurrent Pulmonary Arterial Hypertension following Complete Repair of Congenital Heart Disease,” Pediatric Cardiology 38(7): 1342-1349).

If left permanently, however, the elasticity of an arterial wall notwithstanding, should the pressure increase so that the degree of constriction becomes excessive, the buildup in pressure upstream to the band or valve will eventually cause stretching injury and high ventricular pressure load, if not subannular ventricular hypertrophy (see, for example, Bhattacharya, P. T. and Ellison, M. B. 2022. “Right Ventricular Hypertrophy,” Online, Treasure Island, Florida: StatPearls Publishing Co.; Yalcin, F., Kucukler, N., Cingolani, O., Mbiyangandu, B., Sorensen, L., and 3 others 2019. “Evolution of Ventricular Hypertrophy and Myocardial Mechanics in Physiological and Pathological Hypertrophy,” Online, Journal of Applied Physiology (Bethesda, Maryland) 126(2): 354-362; Kameny R. J., Colglazier, E., Nawaytou, H., Moore, P., Reddy, V. M., Teitel, D., and Fineman, J. R. 2017. “Pushing the Envelope: A Treat and Repair Strategy for Patients with Advanced Pulmonary Hypertension Associated with Congenital Heart Disease,” Pulmonary Circulation 7(3): 747-751; Lopes A. A., Barst, R. J., Haworth, S. G., Rabinovitch, M., Al Dabbagh, M., and 26 others 2014. “Repair of Congenital Heart Disease with Associated Pulmonary Hypertension in Children: What are the Minimal Investigative Procedures? Consensus Statement from the Congenital Heart Disease and Pediatric Task Forces, Pulmonary Vascular Research Institute (PVRI),” Pulmonary Circulation 4(2): 330-341). The introduction of adjustable pulmonary arterial banding made possible the gradual tightening of the band and thus ameliorated the buildup in back pressure that had resulted in subannular injury.

Nevertheless, compared to the instantaneous adaptation of an electrical sphincter with integral means for the release of medication, improved banding remains comparatively crude (see, for example, Changizi, A., Yaghoubi, A., Azarasa, M., Ghaffari, S., and Montazerghaem, H. 2014. “A Study on the Mortality and Complication Rates following Percutaneously Adjustable Pulmonary Artery Banding” Journal of Cardiovascular and Thoracic Research 6(4): 253-255; Sharma, R. 2012. “Pulmonary Artery Banding: Rationale and Possible Indications in the Current Era,” Annals of Pediatric Cardiology 5(1): 40-43). Bands or chokes are placed close enough to the end organ that the velocity and pressure of the blood are unable to recover to the hypertensive values.

By contrast, the intravascular choke acts inside the lumen and is therefore preferred. Similar degeneration of the urothelium has been experienced with artificial sphincters placed to control urinary incontinence. Absent the incorporation of certain protective features, constriction from without further accelerates the degeneration which any cinch would eventually impose. However, as enclosure of the adventitia, even without active constriction, has deleterious consequences, contact is dampened in all three valve types by lining each with a layer, relatively thicker in the sphincteric type, of viscoelastic polyurethane foam, sputter-coated to prevent dissolution.

The foam lining additionally affords some protection against migration, which if anticipated, can be more decisively prevented by providing suture loops 15 at points about the jacket to allow tethering of the jacket to neighboring tissue. If initial discomfort is not spontaneously dispelled due to the fibrous encapsulation that ensues, a topical anesthetic usable internally is directly pipe-targeted to the sites. A drug reservoir implanted subcutaneously in the pectoral region is reserved for the automatic scheduled dispensing by the disorder response system microcontroller or master control microprocessor.

All end actuators in a prosthetic disorder response system incorporate at least one accessory channel (sideline, drugline) to release drugs into their substrate ductus. This action, referred to as direct pipeline targeting, distinguishes such devices and the ductus to which they are attached critically in making it possible to eradicate fouling whether due to the accumulation of clot, crystal, of biofilm that had previously invalidated the use thus of small caliber synthetic materials and valves. Choke servovalves can also be used to both to release therapeutic agents such as stem cells and drugs and synchronize a localized increase in blood pressure to facilitate uptake of the agents or drugs.

While applicable to vessels and more versatile in such application than conventional bands at that, extravascular choke servovalves are presented in U.S. Pat. No. 11,759,186, entitled Ductus Side-entry Jackets and Prosthetic Disorder Response Systems as artificial sphincters for use along the gastrointestinal tract. FIG. 11 in that earlier application shows an electromagnetically operated periductal sphincter, and alternative embodiments such as one that adjusts the sphincter through the turning of a screw by a small motor as in an auto hose clamp are considered obvious.

Lined with foam to comply with peristalsis or the pulse, such valves incorporate at least one accessory channel to allow the automatic, sensor-responsive direct pipeline targeted delivery of drugs to the jacket and substrate native ductus and are dynamically variable in constrictive degree according to the instant need. Not involving other vessels, local pressure reduction by a choke is simplest, not requiring general anesthesia, and more quickly accomplished than alternative methods. The two choke types are therefore useful for shorter term application, such as pressure reduction pending surgical repair or transplantation.

Otherwise, the relief of locally elevated blood pressure is best accomplished by intravascular diversion valves which produce no increase in pressure upstream. Instead, these tap off a small volume of blood for transfer to a larger vessel—from the pulmonary artery to the aorta, for example—for which the added volume and pressure are proportionally negligible. Conventional banding as widely practiced may be adjustable without surgical reentry, but still injures the substrate vessel, inducing atherosclerotic degeneration and calcification, even erosion entirely through the substrate vessel and thrombosis.

Examples of prior art adjustable banding whether absorbable, incorporating an integral inflation tube, catheter balloon adjustable, telemetrically adjustable banding, or ameroid or cellophane banding as used in veterinary practice are not dynamically responsive to the instantaneous level of exertion or posture of the patient (see, for example, Corno, A. F., Kandakure, P. R., Dhannapuneni, R. R., Gladman, G., Venugopal, P., and Alphonso, N. 2013. “Multiple Ventricular Septal Defects: A New Strategy,” Online, Frontiers in Pediatrics 1:16; El-Said, H., Hamzeh, R., Lamberti, J., and Moore, J. 2011. “Catheter Balloon Adjustment of the Pulmonary Artery Band: Feasibility and Safety,” Pediatric Cardiology 32(1): 8-16; (Reyal, J. and Uemoto, S. 2009. “Percutaneously Adjustable Portal Vein Banding Device Could Prevent Post-operative Liver Failure-Artificial Control of Portal Venous Flow is the Key to a New Therapeutic World,” Medical Hypotheses 73(5): 640-650; Davis, M. C. 2008. “Use of a New Implantable Adjustable Pulmonary Artery Banding Device: A Report of Two Patients,” Journal of Extra-corporeal Technology 40(1): 65-67; Bonnet, D., Como, A. F., Sidi, D., Sekarski, N., Beghetti, M., and 6 others 2004. “Early Clinical Results of the Telemetric Adjustable Pulmonary Artery Banding FloWatch-PAB,” Circulation 110(11 Supplement 1): 11158-63). Pneumatic banding is also used in artificial sphincter devices made to counteract urinary incontinence, similar to that of Frederic Foley.

Palliative rather than curative, banding of the pulmonary artery, introduced in 1952 after having been implemented a year or so before publication (Muller, W. H., Jr. and Dammann, J. E. 1952. “Treatment of Certain Congenital Malformations of the Heart by the Creation of Pulmonic Stenosis to Reduce Pulmonary Hypertension and Excessive Pulmonary Blood Flow: A Preliminary Report,” Surgery, Gynecology, and Obstetrics 95:213-219) still accommodates patients unsuited to alternative procedures and continues in widespread use (Dehaki, M. G., Tabaee, A. S., Ahmadabadi, C. A., Ghavidel, A. A., and Omra, G. 2012. “Pulmonary Artery Banding in the Current Era: Is It Still Useful?,” Annals of Pediatric Cardiology 5(1): 36-39), so that eliminating the drawbacks of nonadaptive or ‘dumb’ banding would be of significant benefit. A choke servovalve is a ‘smart’ band.

That banding does afford benefit for selected patients is attested to by efforts to improve bands, such as percutaneous adjustability (Reyal, J. and Uemoto, S. 2009, Op cit.) and absorbable bands (Daley, M., Brizard, C. P., Konstantinov, I. E., Brink, J., Jones, B., and d'Udekem, Y. 2017. “Absorbable Pulmonary Artery Banding: A Strategy for Reducing Reoperations,” European Journal of Cardiothoracic Surgery 51(4): 735-739; Oka, N., Brizard, C. P., Liava'a, M., and d'Udekem, Y. 2011. “Absorbable Pulmonary Arterial Banding: An Optimal Strategy for Muscular or Residual Ventricular Septal Defects,” Journal of Thoracic and Cardiovascular Surgery 141(4): 1081-1082; Bonnet, D., Patkai, J., Tamisier, D., Kachaner, J., Vouhe, P., and Sidi, D. 1999. “A New Strategy for the Surgical Treatment of Aortic Coarctation Associated with Ventricular Septal Defect in infants using an Absorbable Pulmonary Artery Band,” Journal of the American College of Cardiology 34(3): 866-870).

2b(2)(h)(h1). The ‘Tuned’ Shunt

A tuned shunt such as that shown in FIG. 22F is meant to reduce the pressure in a hypertensive vessel by tapping off and moving a volume of blood from the hypertensive to a larger vessel in order to reduce the pressure and thus reduce the injury caused to other organs and tissues. In FIGS. 22C thru 22F (and FIG. 17B), the drugline to the inlet side-entry vascular servovalve is shown as part number 8, while the drugline to the outlet servovalve is shown as part number 8′, and flow that continues through the substrate vessel is shown as part number 41, while that diverted is shown as part number 41′.

In FIG. 22F, diversion chute 18 on the portal vein to the right is that of a pressure sensor-responsive vascular servovalve under the control of a master control microprocessor in which this shunt represents one arm of control in a hierarchical control system to treat portal hypertension, other arms treating etiologically and pathophysiologically related conditions to include that prehepatic, usually consisting of portal or splenic vein thrombosis; that intrahepatic, a form of cirrhosis, and that posthepatic, usually Budd-Chiari syndrome, not to be confused with the three similarly designated types of jaundice. (Miñano, C. and Garcia-Tsao, G. 2010. “Clinical Pharmacology of Portal Hypertension,” Online, Gastroenterology Clinics of North America (Philadelphia, Pennsylvania) 39(3): 681-695; additional references are provided in the section that follows).

A hierarchical control system can treat the different conditions appurtenant of a central disorder such as portal hypertension or can include the treatment of portal hypertension in combination with its causative and secondary complications, or as little if at all related to other comorbidities. In FIG. 22F, the diversion chute on the right apportions flow between the two outlet passages while that on the left is used only to channel the outflow from the portal vein into the inferior vena cava. When a simple flow diversion side-entry jacket as described above in section 2b, entitled Vascular Valves and Servovalves in the Diversion of Blood Flow, in the subsection thereof entitled Simple Flow Diversion Side-entry Jackets, and shown in FIGS. 10F thru 10H, the simple side-entry diversion jacket on the left need not exercise a control or adjustability function but only set a maximum volume for diversion into the inferior vena cava which will be negligible in terms of the total volume of flow through that major vessel.

Unlike a surgical shunt constructed with tissue, which lacking control over the volume flowing through it is passive, immutable, and might be referred to as ‘dumb,’ a feedback-controlled shunt dynamically controls the pressure through the source vessel by adjusting the volume of blood that must be tapped off and diverted to a larger vessel to reduce the elevated pressure. A more comprehensive, such as a hierarchical, fully implanted automatic disorder control system is able not only to counteract the hypertension as the end-symptom, but can effect the collateral treatment of the causative, or etiological, pathophysiological, and sequelary, or consequential, factors as well. That is, in the context of such a system, sufficient treatment comprehends not just constraint of the pressure to within the normal range ameliorative and protective as that is, but to the extent possible, the etiological basis and collateral damage, sequelae, and pathophysiology associated with the disorder.

The ability of a hierarchical control system to treat comorbidities does not adequately express the significance of this ability. If not immediately detected, serious disease will always cause multiple sequelae that strictly speaking, do not represent independent or if later emergent, authentically intercurrent conditions. Also addressed in section 6, entitled Description of the Preferred Embodiments of the Invention, a tuned shunt could be controlled by a fully implanted microcontroller chip as a singular or monomorbid device, but in the more comprehensive treatment of a disease in which a localized hypertensive vessel is a component, can be represented by an arm or branch in a hierarchical control system used to treat multimorbid disease.

In the simpler application, the reporting to the microcontroller of the degree of hypertension by the pressure sensors assigned to the vessel causes the controller to increase the fraction of blood to be tapped off to another vessel. This process of interlocked sensing of hypertension and its counteraction through simultaneous literal pressure relief by tapping off a fraction of blood sufficient to reduce the pressure to within the normal range is continuous.

However, in a hierarchical system, this monomorbid arm is complemented by other arms that target the etiological and pathophysiological concomitants of the disease as a whole to optimize homeostasis across the sum thereof for the specific patient. Such a wider spectrum system not only modulates the volume of blood tapped off but initiates the release of antihypertensive and other medication proportional to the elevation to the associated etiological and pathophysiological concomitants of the disease. To do this, the hierarchical system is provided with drug reservoirs ordinarily implanted subcutaneously in the pectoral region.

In all such treatment, the design consideration of the control system must contemplate the interdependence of the organs and tissues being such that if not aggressively suppressed from the outset, any serious disease will induce collateral injury which will have to be treated as well. The realization that certain disease processes represent a continuum in the development of one continuous disease process has led to combination designations such as the cardiorenal and cardiohepatic syndromes addressed above in section 2a, entitled Background of the Invention. General. An implanted automatic response system can treat each symptom whether part of a causal train, or continuum, or independent and coincidental. In old age when the immune system weakens, both types of disorders are likely to arise.

Provided the etiological cause can be treated, the system will have the potential to provide an authentic cure of it and its sequelary or secondary, and often tertiary disease processes, and advancements in gene and stem cell therapy will bring this objective to realization. A caveat is that good system design seeking to minimize if not eliminate unnecessary complexity, symptoms readily treatable by the patient through adherence to an oral prescription, for example, should be omitted from automated drug delivery. However, the hierarchical control system must still monitor the self-treated symptom as an integral component in each causal chain and separate disease-groups and optimize the treatment of each across the sum thereof for the specific patient.

Insulin over- or underproduction and hyper- or hypothyroidism for example, adversely affect other organs and tissues far removed from the pancreas and thyroid glands, which properly speaking, are part of the same disease process, not “comorbidities” as such, and left untreated, inevitably lead to death (see, for example, Anderson, Z. J., Avula, S., Kumar, A., Sarkar, D., and LaFave, L. 2024. “Graves' Disease-induced Psychosis Refractory to Intensive Medical Management Requiring Non-voluntary Thyroidectomy for Psychosis Resolution: A Case Report,” Cureus (Palo Alto, California) 16(1): e51547; Tomic, D., Shaw, J. E., and Magliano, D. J. 2022. “The Burden and Risks of Emerging Complications of Diabetes Mellitus,” Online, Nature Reviews. Endocrinology (London, England) 18(9): 525-539; Hussain, S. and Chowdhury, T. A. 2019. “The Impact of Comorbidities on the Pharmacological Management of Type 2 Diabetes Mellitus,” Online, Drugs (Auckland, New Zealand) 79(3): 231-242; De Leo, S., Lee, S. Y., and Braverman, L. E. 2016. “Hyperthyroidism,” The Lancet (London, England) 388(10047): 906-918).

A tuned shunt as described herein and shown in FIG. 22F can be fully self-contained and sold as a distinct product, to include a direct drugline from a reservoir implanted subcutaneously in the pectoral region, integral pressure sensor and control microprocessor which can actively vary flow-through in response to the level of pressure elevation to be alleviated. However, a means for reducing hypertension in the portal vein or pulmonary artery is not a cure; rather, the tuned shunt should be used as a component control arm governed by a hierarchical control system that concurrently treats the adverse sequelae caused by the hypertension. As the constellation of symptom-directed control arms effect a reduction in the hypertension, the feedback-driven shunt reduces the volume of blood tapped off.

The suppression of thrombus made possible by the direct targeting of a thrombolytic into the portal vein through a drugline inserted in an accessory channel in the vascular servovalve of the tuned shunt can suppress the formation of thrombus in Budd-Chiari syndrome affected gravidae to reduce if not alleviate the impact upon the fetus in terms of intrauterine growth restriction. Where the condition is not as pronounced, a shunt as a component in a multisymptom-responsive hierarchically controlled disorder response system can be dispensed with and just a simple side-entry diversion jacket drugline under the control of an implanted microcontroller chip used to deliver a thrombolytic.

Fetal or intrauterine growth restriction is addressed above in section 2a, entitled Background of the Invention. General (see also, for example, Gupta, J., Chaudhari, K., and Dave A., and Monica, B. 2023. “Successful Pregnancy Outcome in an Operated Case of Budd-Chiari Syndrome Having Fetal Growth Restriction: A Twisted Tale of Gravid,” Cureus (Palo Alto, California) 15(7): e42745; Wiegers, H., Hamulyak, E. N., Damhuis, S. E., van Duuren, J. R., Darwish Murad, S., and 8 others 2022. “Pregnancy Outcomes in Women with Budd-Chiari Syndrome or Portal Vein Thrombosis—A Multicentre Retrospective Cohort Study, British Journal of Obstetrics and Gynaecology (Oxford, England) 129(4): 608-617).

Not a cure in itself, the tuned shunt is intended to protect tissue affected by the elevated pressure while measures have been instituted to reduce the pressure whereupon the feedback-response shunt will spontaneously reduce the volume of flood tapped off from the hypertensive vessel. It is this combination of components that can improve the overall condition to approximate a cure. Because portal hypertension means that the volume of blood to be processed in the liver, and pulmonary hypertension means that the volume of blood to be oxygenated in the lungs is restricted, merely to reduce the pressure by diverting a volume of blood into a larger vessel does not negate these deficiencies in the normal delivery rates. For this reason, the eradication of the restriction should be treated aggressively to eradicate the causative factor.

End stage cirrhosis warrants a liver transplant, and Budd-Chiari syndrome warrants a thrombectomy, for example. An implanted automatic disorder response system has a central role to play in any such circumstance. Pulmonary hypertension, for example, usually means a need to monitor and treat connective tissue disorders such as” . . . systemic sclerosis, systemic lupus erythematosus, rheumatoid arthritis, Raynaud's disease, and mixed connective tissue disease, numerous drugs, congenital heart disease to include that which causes Eisenmenger's syndrome and systemic-to-pulmonary shunts, hematological disorders to include chronic hemolytic anemia and myeloproliferative disorders, and the systemic diseases that can cause PH include pulmonary Langerhans cell histiocytosis, neurofibromatosis type 1, and sarcoidosis.

Glycogen storage diseases and Gaucher disease are metabolic disorders that can cause pulmonary hypertension.” (Oldroyd, S. H., Manek, G., Sankari, A., and Bhardwaj, A. 2023. “Pulmonary Hypertension,” Online, Treasure Island, Florida: StatPearls Publishing Co.). Posing a need to impose order upon this diversity of causes and associations prompted the decision to propose a hierarchical control system as described in U.S. Pat. No. 11,759,186, entitled Ductus Side-entry Jackets and Prosthetic Disorder Response Systems and in copending application Ser. No. 17/689,880, entitled Prosthetic Disorder Response Systems to deal with the diagnosis and treatment of multimorbid disease.

The medical history and preliminary diagnosis used to reduce the information needed to treat the specific patient, the prescription-program and locations and types of implanted disorder control system sensors needed are determined. Sensors are addressed above in section 2a, entitled Background of the Invention. General. In multimorbid disease where an inordinate number of sensors would be needed to detect an adverse turn to the worse, the number thereof is limited to those that would report a medical emergency were the symptom or symptoms to arise away from the clinic. These too would be the event detectors to prompt telemetric signaling to the clinic.

Portal venous and pulmonary arterial hypertension involve fibrotic degeneration (see, for example, Ghafouri-Fard, S., Askari, A., Shoorei, H., Seify, M., Koohestanidehaghi, Y., and 3 others 2024. “Antioxidant Therapy against TGF-β/SMAD [transforming growth factor beta cytokine signaling protein in the small mother against decapentaplegic tumor suppressor pathway] Involved in Organ Fibrosis,” Online, Journal of Cellular and Molecular Medicine (Oxford, England) 28(2): e18052; Antar, S. A., Ashour, N. A., Marawan, M. E., and Al-Karmalawy, A. A. 2023. “Fibrosis: Types, Effects, Markers, Mechanisms for Disease Progression, and Its Relation with Oxidative Stress, Immunity, and Inflammation,” Online, MDPI [Multidisciplinary Digital Publishing Institute] International Journal of Molecular Sciences (Basel, Switzerland) 24(4): 4004; Kristensen, H., Kimer, N., and Moller S. 2022. “Indications and Methods for Measuring Portal Hypertension in Cirrhosis, Scandinavian Journal of Gastroenterology (London, England)(10): 1149-1157; Ezhilarasan, D. and Lakshmi, T. A 2022. “Molecular Insight into the Role of Antioxidants in Nonalcoholic Fatty Liver Diseases,” Online, Oxidative Medicine and Cellular Longevity (New York, New York) 2022:9233650; Hu, H. H., Chen, D. Q., Wang, Y. N., Feng, Y. L., Cao, G., Vaziri, N. D., and Zhao, Y. Y. 2018. “New Insights into TGF-β/Smad [transforming growth factor beta cytokine signaling protein in the small mother against decapentaplegic tumor suppressor pathway] Signaling in Tissue Fibrosis,” Chemico-biological Interactions (Limerick, Ireland) 292:76-83; Valla, D. C. 2018. “Budd-Chiari Syndrome/Hepatic Venous Outflow Tract Obstruction,” Hepatology International (New York, New York) 12 (Supplement 1): 168-180; Wynn, T. A. and Ramalingam, T. R. 2012. “Mechanisms of Fibrosis: Therapeutic Translation for Fibrotic Disease,” Online, Nature Medicine (New York, New York) 18(7): 1028-1040; Aydinli, M. and Bayraktar, Y. 2007. “Budd-Chiari Syndrome: Etiology, Pathogenesis, and Diagnosis,” Online, World Journal of Gastroenterology (Pleasanton, California) 13(19): 2693-2696).

Not all responses to portal hypertension are medicinal. An implanted automatic response system can be placed before or after a surgical procedure to reduce the elevated pressure. The more recent supplanting of the portacaval with the transjugular intrahepatic portosystemic shunt improved over the prior art. Surgical shunts uncontrollable and without collateral treatment of associated symptoms, an automated feedback-controlled shunt supported by an immediate response system is plainly superior.

With the advent of the implanted automatic response system, surgical shunt repairs should be supplanted leaving other operations to be considered on a case by case basic (see, for example, Qi R Z, Li Z W, Chang Z Y, Chang W H, Zhao W L, and 4 others 2023. “Clinical Efficacy of Total Laparoscopic Splenectomy for Portal Hypertension and Its Influence on Hepatic Hemodynamics and Liver Function,” Online, World Journal of Gastrointestinal Surgery (Pleasanton, California) 15(8): 1684-1692; Henderson, J M, Anderson, C. D. 2020. “The Surgical Treatment of Portal Hypertension,” Online, Clinical Liver Disease (Philadelphia, Pennsylvania) 15 (Supplement 1): S52-S63; Li, J. C. and Henderson, J. M. 2001. “Portal Hypertension,” in Holzheimer, R. G. and Mannick, J. A. Surgical Treatment, Online at https://www.ncbi.nlm.nih.gov/books/NBK6973/#).

Similarly, surgical and interventional treatment for pulmonary hypertension can be applied before or after the placement of an implanted automatic response control system; however, the expectation that a fixed shunt could support the patient with anything approaching a comparable degree of pertinent therapy extended to the etiological and sequelary factors associated with the disorder would appear incontestable. This understood, most surgical and interventional procedures to ameliorate pulmonary hypertension are considerably potentiated when combined with an implanted automatic response system.

A pulmonary endarterectomy would appear less actively curative than the ischemia free placement of an inline coupling jacket-secured prosthesis as described above in section 2b(2)(c), entitled Vascular Segment Prostheses: Inline Coupling Jackets and Bridging, or Tie-lines and its subsidiary sections, where the prosthesis in an adult is tubular but for a growing child, made with the expandable structure delineated above in section 2b(2)(f), entitled Expandable Structure to Accommodate Growth and Self-adjust in Size in Vascular Segment Prostheses, Lines, and Confluence Conduits Placed in Children (see, for example, Oldroyd, S. H., Manek, G., Sankari, A., and Bhardwaj, A. 2023, Op cit. above in this section; Stącel, T., Latos, M., Urlik, M., Necki, M., Antończyk, R., and 3 others 2021. “Interventional and Surgical Treatments for Pulmonary Arterial Hypertension,” Online, MDPI [Multidisciplinary Digital Publishing Institute] Journal of Clinical Medicine (Basel, Switzerland) 10(15): 3326).

Both the portal venous and pulmonary arterial pressures vary in health before the complications and variables added by disease are considered. Portal hypertension is considered to commence at above 9 millimeters mercury (9 mmHg); however, following a meal, the portal blood pressure and liver stiffness increase into the range associated with impairment.

Liver stiffness is indictive of fibrosis and cirrhosis which due to chronic liver disease can induce as well as be induced by portal hypertension Oliver, T. I., Sharma, B., and John, S. 2023. “Portal Hypertension,” Online, Treasure Island, Florida: StatPearls [Internet]. StatPearls Publishing Co.; Ortega-Ribera, M., Gibert-Ramos, A., Abad-Jorda, L., Magaz, M., Telle,z L., and 13 others 2023. “Increased Sinusoidal Pressure Impairs Liver Endothelial Mechanosensing, Uncovering Novel Biomarkers of Portal Hypertension,” (Online) JHEP [Journal of Hepatology] Reports: Innovation in Hepatology (Amsterdam, North Holland, Netherlands) 5(6): 100722; Iwakiri, Y. and Trebicka, J. 2021. “Portal Hypertension in Cirrhosis: Pathophysiological Mechanisms and Therapy,” (Online) JHEP [Journal of Hepatology] Reports: Innovation in Hepatology (Amsterdam, North Holland, Netherlands) 34): 100316; Lunova, M., Frankova, S., Gottfriedova, H., Senkerikova, R., Neroldova, M., and 7 others 2021. “Portal Hypertension is the Main Driver of Liver Stiffness in Advanced Liver Cirrhosis,” Online, Physiological Research (Praha, Czech Republic) 70(4): 563-577; Alvarez, D., Orozco, F., Mella, J. M., Anders, M., Antinucci, F., and Mastai, R. 2015. “Meal Ingestion Markedly Increases Liver Stiffness Suggesting the Need for Liver Stiffness Determination in Fasting Conditions,” [Spanish and English] Online, Gastroenterologia y Hepatologia. (Madrid, Spain) 38(7): 431-435; Iwakiri, Y. 2014. “Pathophysiology of Portal Hypertension,” Online, Clinics in Liver Disease (Philadelphia, Pennsylvania) 18(2): 281-291; Berzigotti, A., De Gottardi, A., Vukotic, R., Siramolpiwat, S., Abraldes, J. G., Garcia-Pagan, J. C., and Bosch, J. 2013. “Effect of Meal Ingestion on Liver Stiffness in Patients with Cirrhosis and Portal Hypertension,” Online, PLoS [Public Library of Science] One (San Francisco, California) 8(3): e58742; Gaiani, S., Bolondi, L., Li Bassi, S., Santi, V., Zironi, G., and Barbara, L. 1989. “Effect of Meal on Portal Hemodynamics in Healthy Humans and in Patients with Chronic Liver Disease,” Online, Hepatology (Baltimore, Maryland) 9(6): 815-819).

Due to the number of variables that pertain to most patients, the relationship of kidney transplantation to these findings is unclear (see, for example, Dodge, J. L., Lee, B. T., Kassem, A. C. Z., Biggins S W, Rana P A, and 3 others 2023. “The Conundrum of Patients with Compensated Cirrhosis Requiring Kidney Transplantation: Kidney Alone or Simultaneous Liver Kidney Transplantation,” Transplantation (Hagerstown, Maryland) 107(2): 429-437; Machado, P., Gupta, I., Fenkel, J. M., Gummadi, S., Stanczak, M., and 7 others 2022. “Ultrasound Pressure Estimation for Diagnosing Portal Hypertension in Patients Undergoing Dialysis for Chronic Kidney Disease,” Online, Journal of Ultrasound in Medicine (Oxford, England) 41(9): 2181-2189; Chmielewski, J., Lewandowski, R. J., and Maddur, H. 2018. “Hepatorenal Syndrome: Physiology, Diagnosis, and Management,” Online, Seminars in Interventional Radiology (New York, New York) 35(3): 194-197; Shah, N. J. and Russo, M. W. 2017. “Is It Time to Rethink Combined Liver-Kidney Transplant in Hepatitis C Patients with Advanced Fibrosis?,” Online, World Journal of Hepatology (Beijing, China) 9(5): 288-292; Parsikia, A., Campos, S., Khanmoradi, K., Pang, J., Balasubramanian, M., Zaki, R., and Ortiz, J. 2015. “Equal 3-Year Outcomes for Kidney Transplantation Alone in HCV [hepatitis C virus]-positive Patients with Cirrhosis, Online, International Surgery (Chicago, Illinois) 100(1): 142-154; Dickerman, R. M., Niederhuber, J. E., Eigenbrodt, E., and Fry, W. J. 1978. “Portal Hypertension Following Renal Transplantation,” Surgery (St. Louis, Missouri) 84(3): 322-328).

Pulmonary hypertension responds to exertion but not without posing ambiguities as to the degree of elevation that falls within the normal range (see, for example, Kovacs, G., Berghold, A., Scheidl, S., and Olschewski, H. 2009. “Pulmonary Arterial Pressure during Rest and Exercise in Healthy Subjects: A Systematic Review,” European Respiratory Journal (Sheffield, England) 34(4): 888-894).

In FIG. 22F, the outflow conduit shown as part number 196 in a mostly grown or an adult is a simple tube; in and infant, it is an expandable structured conduit. This is done by tapping off a volume of blood from the hypertensive vessel to bring its pressure to within the normal range.

The blood diverted is passed into a larger vessel where the increase in volume would less if not at all exert an increase in pressure sufficient to cause equivalent problems. In pulmonary hypertension, a portion of the deoxygenated blood flowing through it is diverted from the pulmonary trunk, or main pulmonary artery, and added to the deoxygenated blood flowing through the somewhat larger superior vena cava.

Such a shunt is referred to as ‘tuned’ because rather than fixed, or ‘dumb,’ as is a surgical shunt, the tuned shunt is dynamic in continuous or intermittent adjustment in pressure relief effected by the vascular servovalve at its inlet under sensor-driven negative feedback. In portal hypertension, the excess pressure is relieved by diverting the volume of blood flowing through the portal vein to reduce to pressure to within the normal range. Here one is confronted with the complication that the blood diverted contains the nutrient and neurotoxic products of digestion.

In monomorbid disease, a tuned shunt is controlled by a microcontroller implanted in the pectoral area. In comorbid or multimorbid disease, control is by a similarly positioned master control microprocessor. In either case, the controller responds to one or more blood pressure sensors positioned at the site of excessive blood pressure. Tune shunts can be used to correct the lack of appropriate heart action following exercise, for example, where the innate sensors in the aorta and carotids have become dysfunctional

Anastomosis bypasses such as shown in FIGS. 22B thru 22E, 32D, and 32F and tuned shunts such as shown in FIG. 22F differ functionally in that the former allow switching between flow through an anastomosis and flow that bypasses the anastomosis along the same vessel, while the latter allows the control of flow between two different vessels. When structurally the same with servovalve-controlled diversion chutes, the flow-through cross-section of the bypass or tuned shunt respectively is made continuously variable. However, when an anastomosis bypass uses a plunger solenoid-controlled diversion chute to suddenly open or close the bypass, the two differ structurally.

Untreated, all of the toxins, to include ammonia as the suspect cause of portosystemic encephalopathy, the level thereof reduced notwithstanding, its buildup in the systemic circulation as the probable cause for hepatic encephalopathy remains in the circulation. However, means already exist to deal with this probem, and drugs that can ameliorate the problem or can do so when delivered in their post-liver metabolized form can be directly pipe-targeted to the shunt servovalve and/or the gut.

Currently, efforts to develop a liver-on-a-chip remain experimental (see, for example, Liu, M., Xiang, Y., Yang, Y., Long, X., Xiao, Z., and 5 others 2022. “State-of-the-art Advancements in Liver-on-a-chip ‘(LOC): Integrated Biosensors for LOC,” Biosensors and Bioelectronics 218:114758; Yang, Z., Liu, X., Cribbin, E. M., Kim, A. M., Li, J. J., and Yong, K. T., 2022. “Liver-on-a-chip: Considerations, Advances, and Beyond,” Biomicrofluidics 16(6): 061502). As do almost all side-entry jackets and vascular servovalves, those incorporated into tuned shunts are equipped with at least one drugline for the direct pipe-targeting of drugs to the treatment site.

The section that follows, 2b(2)(h)(h2), entitled The ‘Tuned’ Shunt in the Relief of Portal Venous Hypertension, addresses the combination of a tuned shunt to decompress the hepatic portal vein and an implanted magnetically based implanted aphereter/dialyzer described in U.S. Pat. No. 11,759,186, entitled Ductus Side-entry Jackets and Prosthetic Disorder Response Systems, to extract the nocuous constituents from the bloodstream which lead to portopulmonary hypertension, bleeding varices, and several other serious complications.

Hepatic encephalopathy is also a common occurrence following the placement of a transjugular intrahepatic portosystemic shunt. If applied early enough, pressure relief in portal hypertension should reduce the rate of liver damage and to this extent reduce the degree of hepatic encephalopathy which is its cause. Diverting a portion of the blood flowing through the portal vein and passing it directly into the lower vena cava is clearly a partial, supplementary treatment to the use of drugs or agents such as lactulose, and if there is a bacterial infection, then from a long list of possible drugs, a penicillin, cephalosporin, tetracycline, or aminoglycoside, likely having already been prescribed to treat the problem.

The very cautious use of a propofol pump-which except for electroencephalographic electrodes applied to the scalp in preliminary diagnostic examination can be made subject to control by a prosthetic disorder response system—as might almost any other diagnostic or therapeutic apparatus used in the clinic using conventional electronic means—has been found to control nonconvulsive status epilepticus (see, for example, Ayele, T., Ezeh E., Al-Qawasmi, L., Ugonabo, O. S., Saylor, J., and Dial, L. 2022. “Diagnosed by Reversibility: Unmasking Propofol-related Infusion Syndrome in a Critically Ill Elderly Male,” Online, Cureus (Palo Alto, California) 14(3): e23505; Folino, T. B., Muco, E., Safadi, A. O., and Parks, L. J. 2022. “Propofol,” Online, Treasure Island, Florida: StatPearls Publishing Co.; Secor, T., Safadi, A. O., and Gunderson, S. 2022. “Propofol Toxicity,” Online, Treasure Island, Florida: StatPearls Publishing Co.; Shi, F., Chen, Y., Li, H., Zhang, Y., and Zhao, T. 2022. “Efficacy and Safety of Remimazolam Tosilate versus Propofol for General Anesthesia in Cirrhotic Patients Undergoing Endoscopic Variceal Ligation,” Online, International Journal of General Medicine 15:583-591; Singh, A. and Anjankar, A. P. 2022. “Propofol-related Infusion Syndrome: A Clinical Review,” Online, Cureus (Palo Alto, California) 14(10): e30383; Tang, Y., Yang, X, Yu, Y., Shu, H., Xu, J., and 4 others 2022. “Remimazolam versus Traditional Sedatives for Procedural Sedation: A Systematic Review and Meta-analysis of Efficacy and Safety Outcomes,” Minerva Anestesiologica 88(11): 939-949; Jeong, J., Jung, K., Seo, K. I., Yun, B. C., Han, B. H., Lee, S. U., and Moon, W. 2020. “Propofol Alone Prevents Worsening Hepatic Encephalopathy Rather than Midazolam Alone or Combined Sedation after Esophagogastroduodenoscopy in Compensated or Decompensated Cirrhotic Patients,” Online, European Journal of Gastroenterology and Hepatology 32(8): 1054-1061; Hor, S. Chen, C. Y., and Tsai, S. T. 2019. “Propofol Pump Controls Nonconvulsive Status Epilepticus in a Hepatic Encephalopathy Patient: A Case Report,” Online, World Journal of Clinical Cases 7(18): 2831-2837; Lin, C., Darling, C., and Tsui, B. C. H. 2019. “Practical Regional Anesthesia Guide for Elderly Patients,” Drugs and Aging 36(3): 213-234; Sprinks, J., Worcester, F., Breedon, P., Watts, P., Hewson, D., and Bedforth, N. 2019. “A Prototype Patient-maintained Propofol Sedation System Using Target Controlled Infusion for Primary Lower-limb Arthroplasty,” Online, Journal of Medical Systems 43(8): 247; Watanabe, K., Hikichi, T., Takagi, T., Suzuki, R., Nakamura, J., and 9 others 2018. “Propofol is a More Effective and Safer Sedative Agent than Midazolam in Endoscopic Injection Sclerotherapy for Esophageal Varices in Patients with Liver Cirrhosis: A Randomized Controlled Trial,” Online, Fukushima Journal of Medical Science 64(3): 133-141; Vidaurre, J., Gedela, S., and Yarosz, S. 2017. “Drugs and Liver Disease,” Online, Pediatric Neurology 77:23-36; Gotoda, T., Okada, H., Hori, K., Kawahara, Y., Iwamuro, M. and 7 others 2016. “Propofol Sedation with a Target-controlled Infusion Pump and Bispectral Index Monitoring System in Elderly Patients during a Complex Upper Endoscopy Procedure,” Gastrointestinal Endoscopy 83(4): 756-764; Vasile, B., Rasulo. F., Candiani. A., and Latronico, N. 2003. “The Pathophysiology of Propofol Infusion Syndrome: A Simple Name for a Complex Syndrome,” Intensive Care Medicine 29(9): 1417-1425).

“If needed, the effects of remimazolam can be reversed by flumazenil, which allows prompt termination of sedation,” (Hu, Q., Liu, X., Wen, C., Li, D., and Lei, X. 2022. “Remimazolam: An Updated Review of a New Sedative and Anesthetic,” Online, Drug Design, Development, and Therapy 16:3957-3974; Absalom, A. R., Glen, J. I., Zwart. G. J., Schnider, T. W., and Struys, M. M. 2016. “Target-controlled Infusion: A Mature Technology,” Online, Anesthesia and Analgesia 122(1): 70-78; Kim, K. M/2022. “Remimazolam: Pharmacological Characteristics and Clinical Applications in Anesthesiology,” Online, Anesthesia and Pain Medicine (Seoul, South Korea0 17(1): 1-11).

Conventional means are able to reverse the syndrome, thus overcoming the harm in the perpetuation of toxins in the systematic circulation as the essential shortcoming inherent in pressure relief with the aid of a tuned shunt with or without a simple side-entry jacket to directly target drugs into the gut (see, for example, Ahmed, S., Premkumar, M. Dhiman, R. K., Kulkarni, A. V., Imram, R., and 7 others 2022. “Combined PEG3350 Plus Lactulose Results in Early Resolution of Hepatic Encephalopathy and Improved 28-day Survival in Acute-on-chronic Liver Failure,” Journal of Clinical Gastroenterology 56(1): e11-e19; Feng, X., Li, X., Zhang, X., Chen, W., Tian, Y., and 4 others 2022. “Hepatic Encephalopathy in Cirrhotic Patients and Risk of Small Intestinal Bacterial Overgrowth,” Online, BioMed Research International 2022:2469513; Hollat, G. J., Suhail, F. K., Adhami. T., and John, S. 2022. “Evidence-based Approach to Management of Hepatic Encephalopathy in Adults,” Online, World Journal of Hepatology 14(4): 670-681; Lee, P. C., Lee, K. C., Yang, T. C., Lu, H. S., Cheng. T. Y., and 11 others 2022. “Sarcopenia-relatied Gut Microbial Changes are Associated with the Risk of Complications in People with Cirrhosis,” Online, Journal of Hepatology 5(1): 100619; Mandiga. P., Foris, L. A., and Bolu, P. C. 2022. “Hepatic Encephalopathy,” Online, Treasure Island, Florida: StatPearls Publishing Co.; Menon, V., Ransing, R., and Praharaj, S. K., 2022. “Management of Psychiatric Disorders in Patients with Hepatic and Gastrointestinal Diseases,” Online, Indian Journal of Psychiatry 64 (Supplement 2): S379-S393; Miwa, T., Hanai. T., Imai, K., Takai, K. Shiraki, M., and 5 others 2022. “Effects of Rifaximin on Circulating Albumin Structures and Serum Ammonia Levels in Patients with Liver Cirrhosis: A Preliminary Study,” Online, MDPI [Multidisciplinary Digital Publishing Institute] Journal of Clinical Medicine (Basel, Switzerland) 11(24): 7318; Mukherjee. S. and John S. 2022. “Lactulose,” Online, Treasure Island, Florida: StatPearls Publishing Co.; Nguyen, A., Butt. M. A., Upadhyay, S., Sheikh, A. B., and Shekhar, R. 2022. “Non-convulsive Status Epilepticus in Hepatic Encephalopathy: A Case Series and Review of the Literature,” Online, European Journal of Case Reports in Internal Medicine 9(2): 003179; Olivero, M., Gagliardi, D., Costamagna, G., Velardo, D., Magri, F., and 5 others 2022. “Newly Diagnosed Hepatic Encephalopathy Presenting as Non-convulsive Status Epilepticus: A Case Report and Literature Review,” Online, Frontiers in Neurology 13:880068; Premkumar, M. and Anand, A. C. 2022. “Overview of Complications in Cirrhosis,” Journal of Clinical and Experimental Hepatology 12(4): 1150-1174; Sharma, K., Akre, S., Chakole, S., and Wanjari, M. B. 2022. “Hepatic Encephalopathy and Treatment Modalities: A Review Article,” Online, Cureus (Palo Alto, California) 14(8) e28016; Veirup, N. and Kyriakopoulos, C. 2022. “Neomycin,” Online, Treasure Island, Florida: StatPearls Publishing Co.; Won, S.-M., Oh, K. K., Gupta, H., Ganesan, R., and 13 others 2022. “The Link between Gut Microbiota and Hepatic Encephalopathy,” Online, MDPI [Multidisciplinary Digital Publishing Institute] International Journal of Molecular Sciences (Basel, Switzerland) 23(16): 8999; Bajaj. J. S., Sikaroodi, M., Shamsaddini, A., Henseler, Z., Santiago-Rodriguez, T., and 8 others 2021. “Interaction of Bacterial Metagenome and Virome in Patients with Cirrhosis and Hepatic Encephalopathy,” British Journal of Medicine Gut 70(6): 1162-1173; Bloom, P. P., Tapper, E. B., Young. V. B., and Lok, A. S. 2021. “Microbiome Therapeutics for Hepatic Encephalopathy,” Online, Journal of Hepatology 75(6): 1452-1464; Saboo. K., Shamsaddini, A., Iyer, M. V., Hu, C., Fagan, A., and 8 others 2021. “Sex is Associated with Differences in Gut Microbial Composition and Function in Hepatic Encephalopathy, Online, Journal of Hepatology 74(1): 80-88; Shamsaddini. A., Gillevet, P. M., Acharya, C., Fagan A., Gavis, E., and 7 others 2021. “Impact of Antibiotic Resistance Genes in Gut Microbiome of Patients with Cirrhosis,” Online, Gastroenterology 161(2): 508-521: Trebicka, J., Macnaughtan, J., Schnabl, B., Shawcross, D. L., and Bajaj. J. S. 2021. “The Microbiota in Cirrhosis and Its Role in Hepatic Decompensation,” Online, Journal of Hepatology 75 (Supplement 1): S67-S81; Rose, C. F., Amodio, P., Bajaj. J. S., Dhiman, R. K., Montagnese, S., and 3 others 2020. “Hepatic Encephalopathy: Novel Insights into Classification, Pathophysiology, and Therapy,” Journal of Hepatology 73(6): 1526-1547; Peters, J., Vijiaratnam, N., Wong, J. Z. W., Jitpiriyaroj. S., Chandra, R. V., and Kempster, P. A. 2019. “Propensity for Seizure-related Cortical Laminar Necrosis in Hepatic Encephalopathy,” Online, Epilepsy and Behavior Reports 12:100348; Newey, C. R. George, P., Sarwal. A., So, N., and Hantus, S. 2018. “Electro-radiological Observations of Grade III/IV Hepatic Encephalopathy Patients with Seizures,” Neurocritical Care 28(1): 97-103; Elwir, S. and Rahimi. R. S. 2017. “Hepatic Encephalopathy: An Update on the Pathophysiology and Therapeutic Options,” Online, Journal of Clinical and Translational Hepatology 5(2): 142-151; Falavigna, M., Kieling. C., Wolff, F. H., Medeiros, L. R. F., and Cheinquer, H. 2017, “Antibiotics for Hepatic Encephalopathy,” Online, Cochrane Database of Systematic Reviews 2017(7): CD006314; Ferenci. P. 2017. “Hepatic Encephalopathy,” Online, Gastroenterology Report (Oxford, England) 5(2): 138-147; Rudler, M., Marois. C., Weiss, N., Thabut, D., and Navarro, V. 2017. “Status Epilepticus in Patients with Cirrhosis: How to Avoid Misdiagnosis in Patient with Hepatic Encephalopathy,” Online, Seizure-European Journal of Epilepsy 45:192-197; Rahimi. R. S., Singal, A. G., Cuthbert, J. A., and Rockey, D. C. 2014. “Lactulose vs Polyethylene Glycol 3350-electrolyte Solution for Treatment of Overt Hepatic Encephalopathy: The HELP [Hepatic Encephalopathy: Lactulose vs Polyethylene Glycol 3350-electrolyte] Randomized Clinical Trial,” Journal of the American Medical Society Internal Medicine 174(11): 1727-1733; Patidar, K. R. and Bajaj. J. S. 2013. “Antibiotics for the Treatment of Hepatic Encephalopathy,” Online, Metabolic Brain Disease 28(2): 307-312′ Bajaj. J. S., Ridion, J. M., Hylemon, P. B., Thacker, L. R., Heuman, D. M., and 3 others 2012. “Linkage of Gut Microbiome with Cognition in Hepatic Encephalopathy,” Online, American Journal of Physiology. Gastrointestinal and Liver Physiology 302(1): G168-G175; Ryan, J. M. and Shawcross, D. L. 2011. “Hepatic Encephalopathy,” Medicine, Management Problems in Liver Disease 30(10): 617-620).

Where appropriate, such drugs can be delivered directly into the shunt through its servovalve drugline, or accessory channel, part number 8 in the drawing figures. Drugs that function not as prodrugs requiring first pass through the liver but having been converted into their post-liver form can also be released directly into the portal vein, for example, through a simple side-entry flow diversion valve jacket. While pressure relief in vessels should ameliorate if not eliminate the damage caused by hypertension, an actual cure for portal and pulmonary hypertension would reduce if not end the hypertension by successfully treating the often extrahepatic primary cause or causes for the problem.

A more etiological approach is made difficult by the numerous causes, congenital and/or acquired, which often pose difficulty even to obtaining a clear diagnosis. In those with severe disease, a liver transplant, addressed above in section 2b(1)(b)(b7), entitled Metered Compound Vascular Bypass, or Switched-organ, Liver or Heart-Liver Transplantation, often achieves survival for about five years.

A tuned shunt comprises:
1. A vascular servovalve with one or more thin-film pressure sensors, for example, along its internal surface positioned in encircling relation to a hypertensive artery.
2. A length of tubing to conduct a volume of blood diverted by the vascular servovalve surrounding the hypertensive artery and lead it into a larger vessel.
3. A simple side-entry diversion valve encircling the larger receiving vessel at the other end to connect the tube at the other end and serve as inlet for the blood led through it.
4. A microcontroller chip suitably encased and implanted in the patient to accept the pressure data from the servovalve used to continuously or intermittently control the volume of blood the servovalve is to divert, from a hypertensive vessel in order to bring its pressure to within the normal range through negative feedback.
5. The electrical conductors to connect the sensors to the microcontroller and the microcontroller to the vascular servovalve.

In such a limited application, the microcontroller can be positioned close enough to the servovalve to eliminate the need for radio such as Bluetooth connection. In a more comprehensive system, the microcontroller would represent a node in a hierarchical control system administered by a master control microprocessor.

Placement is as depicted in the drawing figures, the servovalve placed in encircling relation to the substrate native ductus to divert flow out through a synthetic tube. As shown in FIG. 15A, the receiving end is a servovalve no different than that at the entry but positioned in reverse. The chip is programmed to execute the control program, which consists of adjusting the shunt flow-through volume as the need therefor indicated by local sensors dictates and of delivering medication directly into the shunt as indicated by other sensors implanted locally and in the locations of sequelary consequences.

By ‘tuned’ shunt is meant that flow rate through the shunt is automatically controlled in direct response to the instant condition. The tuned shunt is intended to ameliorate the adverse sequelae associated with ventriculoperitoneal, portacaval, or any other type conventional shunt. In a majority of cases, such sequelae consist primarily of infection and thrombosis, and since every ‘tuned’ shunt has at least one drugline, or accessory channel, the direct pipe-targeted delivery of antimicrobials and antithrombotics, for example, can be administered by a fully implanted prosthetic disorder response system.

As addressed here, shunts are used to move blood from one vessel to another, sometimes even to satisfy an inadequacy of volume and pressure at the receiving end, but also to relieve an excess of volume and pressure at the end drained. Always responsive to a primary cause, best practice calls for correction of that cause whenever possible, not merely treating the symptoms; however, certain congenital defects of anatomy and/or physiology, accidental trauma, and damage caused by disease may make this impossible. If irreparably or irremediably defective, such as a cirrhotic liver which blocks the passage of blood so that pressure builds up in the portal vein to dilate the esophageal veins, risking variceal rupture, hypovolemic shock, hepatic encephalopathy, and death, for example, the liver is best replaced.

Sensors to detect bacteria, viruses, neoplasms, thrombus, urinary crystal, inflammation, infection, and rejection, for example, are addressed above in section 2a, entitled Background. General, in the subsection thereof entitled Sensors. Drawing a volume of blood from the hypertensive vessel for transport to another vessel, the shunt is positioned downstream to the hypertensive vessel. The direct pipe-targeting of medication to the hypertensive vessel itself or to the destination vessel for receipt of the tapped volume is through a drugline connected to that vessel by a simple side-entry valve. That is, drug volume controlled by the implanted automatic prosthetic disorder response system at the drug reservoir, not at the target ductus as to necessitate a servovalve, drug delivery into the source or destination hypertensive artery is through an ordinary side-entry valve.

The servovalve at the entry to the tuned shunt incorporates a drugline inserted into its accessory channel, which allows the automatic control system upon detecting the need to directly pipe-target an anticoagulant or crystal counteractant to clear the shunt itself, or antimicrobial, antithrombotic, immunosuppressive, or a local antihypertensive in the form of a topical vasodilator, for example, to treat the destination vessel if necessary (see, for example, Kong, E., Shu, L., Yuan, C., Wang, J., Wu, F., Yuan, H., and Feng, X. 2022. “Efficacy Assessment of Phentolamine Accompanied by Lidocaine Subcutaneously under Ultrasound Guidance on Radial Artery Catheterization in Pediatric Patients,” Online, BioMed Research International 2022:6554993; Duman, I., Tanyeli, O., Dereli, Y., Oltulu, P., Toy, H., and Sahin, A. S. 2021. “The Effects of Botulinum Toxin A and Papaverine on Human Saphenous Vein and Internal Mammary Artery Grafts: An in Vitro Study,” Online, Archives of Medical Science 18(6): 1607-1616; Seth, R., Badran, K. W., Cedrars, E., Plonowska, K., Benjamin, T., and 4 others 2021. “Vasodilation by Verapamil-Nitroglycerin Solution in Microvascular Surgery,” Online, Otolaryngology-Head and Neck Surgery 164(1): 104-109; Jang, Y. E., Ji, S. H., Kim, E. H., Lee, J. H., Kim, H. S., Mossad, E. B., and Kim, J. T. 2020. “Subcutaneous Nitroglycerin for Radial Arterial Catheterization in Pediatric Patients: A Randomized Controlled Trial,” Online, Anesthesiology 133(1): 53-63; Ricci, J. A., Singhal, D., Fukudome, E. Y., Tobias, A. M., Lin, S. J., and Lee, B. T. 2018. “Topical Nitroglycerin for the Treatment of Intraoperative Microsurgical Vasospasm,” Microsurgery 38(5): 524-529; Rinkinen, J. and Halvorson, E. G. 2017. “Topical Vasodilators in Microsurgery: What is the Evidence?” Journal of Reconstructive Microsurgery 33(1): 1-7; Vargas, C. R., Iorio, M. L, and Lee, B. T. 2015. “A Systematic Review of Topical Vasodilators for the Treatment of Intraoperative Vasospasm in Reconstructive Microsurgery,” Plastic and Reconstructive Surgery 136(2): 411-422; Mussa, S., Guzik, T. J., Black, E., Dipp, M. A., Channon, K. M., and Taggart, D. P. 2003, “Comparative Efficacies and Durations of Action of Phenoxybenzamine, Verapamil/Nitroglycerin Solution, and Papaverine as Topical Antispasmodics for Radial Artery Coronary Bypass Grafting,” Online, Journal of Thoracic and Cardiovascular Surgery 126(6): 1798-1805).

The ability to target medication to the source vessel or the shunt makes such a shunt critically more capable than any valved shunts already available. When the site is upstream to as to precede passage through the liver, an agent ordinarily administered as a prodrug is formulated in its post-liver metabolized form, any residue passing into the systemic circulation in little more than a trace amount. Analogous to cerebral ventricle drainage shunts, marketed shunts do not incorporate a drugline, or accessory channel, to directly pipe-target antimicrobial, anti-inflammatory, immunosuppressive, and/or antineoplastic medication, for example, into the shunt.

This concept is compatible with existing shunts to include transjugular intrahepatic portosystemic shunts where the added support of an implanted prosthetic disorder response system equipped with sensors and prepositioned druglines deployed to different sites of secondary and teriatary adverse sequelae can ameliorate common problems encountered with conventional shunts. Obstruction to drainage no less results in a buildup of upstream, or retrograde, pressure.

A fully implanted automatic drug delivery system as described in U.S. Pat. No. 11,759,186, entitled Ductus Side-entry Jackets and Prosthetic Disorder Response Systems, can directly target drugs to the site of obstruction to palliate and to this extent ameliorate the condition. Unlike a semipermanent if not permanent central line, such a system, replenished percutaneously by injection through a small subcutaneous port leading from a subcutaneous drug reservoir to the target vessel through a side-entry jacket with supportive drug delivery accessory channels, is less susceptible to contamination and clot.

When not life-threatening, or until a replacement organ becomes available, the combination of directly targeted medication such as Remodulin® (United Therapeutics, Silver Spring, Maryland) treprostinil injection/infusion and relief of pressure by diversion of a portion of the blood to one or more large arteries through a controllable shunt can ameliorate if not correct the secondary but no less dangerous condition. Along with numerous other causes, portal and pulmonary hypertension can result from downstream resistance to flow where the resistance is primary and the hypertension secondary.

Collateral circulation is an example of spontaneous native or endogenous shunting, a spontaneous adaptive response that compensates for an insufficiency or a misdirection of perfusion by a normally formed and situated blood supply. In an athlete with hyperlipidemia, collateral circulation can achieve sufficient perfusion so that angina is not experienced, there is negligible risk of a myocardial infarction, and provided dyslipidemia-suppressing medication is taken to prevent the collaterals from becoming occluded, coronary artery bypass grafting may be unnecessary.

However, spontaneous collaterals often prove inadequate; they can anastomose in locations that create other health problems or develop too slowly to prevent end-stage disease. Collaterals develop to compensate for pulmonary and portal hypertension, but do not negate the need for the addition of a surgical collateral, that is, a bypass. In contrast to spontaneous shunts or collaterals, a femorofemoral crossover graft to provide better perfusion of a leg and ventriculoperitoneal, ventriculoatrial, or lumboperitoneal shunts to drain excess cerebrospinal fluid in a hydrocephalic infant rarely develops spontaneously and most often requires to be created surgically.

Where the spontaneous formation of a fistula or defensive rupture of a ventricle to create an internal drainage pathway to compensate for the buildup in pressure (which can be viewed as another type of spontaneously formed shunt) would prove beneficial by serving to drain the excess fluid and eliminate the need for the surgical placement of a shunt, a natural fistula is rarely formed, or if it is, the fistula is likely to open into a nonphysiological space for the containment of cerebrospinal fluid (see, for example, Fowler, J. B., de Jesus. O., and Mesfin, F. B. 2022. “Ventriculoperitoneal Shunt” Online, Treasure Island, Florida: StatPearls Publishing Co.; Desai, V. R., Sadrameli, S. S., Jenson, A. V., Asante, S. K., Daniels B., Trask, T. W. and Britz, G. H. 2020. “Ventriculoperitoneal Shunt Complications in an Adult Population: A Comparison of Various Shunt Designs to Prevent Overdrainage,” Online, Surgical Neurology International 11:269; Krishna Pillai. M., Kariyattil, R., Rubene. L., Kumar, R., Govindaraju, V., and Kochummen, K. 2018. “Spontaneous Ventriculostomy into the Subdural Space in a Neonate with Congenital Hydrocephalus,” Pediatric Neurosurgery 53(3): 188-192; Ogrenci, A., Ekşi, M. S., and Koban, O. 2016. “Spontaneous Third Ventriculostomy 8 Years after Diagnosis of Obstructive Hydrocephalus,” Child's Nervous System 32(9): 1727-1730; Gallia, G. L. and Teo. C. 2008. “Spontaneous Third Ventriculocisternostomy in an Infant with Obstructive Hydrocephalus,” Journal of Neurosurgery. Pediatrics 1(6): 477-480; Deniz, F. E., Ece, K., Celik, O., Akalan, N., and Firat, M. M. 2008. “Spontaneous Third Ventriculostomy in Chronic Obstructive Hydrocephalus,” Child's Nervous System 24(5): 633-634; Kim, L. J., Feiz-Erfan, I., Clatterbuck, R. E., and Spetzler, R. F. 2005. “Spontaneous Ventriculostomy in a Patient with Obstructive Hydrocephalus,” Acta Neurochirurgica (Vienna, Austria) 147(2): 219-220; Rovira, A., Capellades, J., Grive, E., Poca, M. A., Pedraza, S., Sahuquillo, J., and Rodriguez-Baeza, A. 1999. “Spontaneous Ventriculostomy: Report of Three Cases Revealed by Flow-sensitive Phase-contrast Cine MR [magnetic resonance] Imaging,” American Journal of Neuroradiology 20(9): 1647-1652).

Contrariwise, the formation of a fistula after a surgical shunt has been placed to complicate the correction is not rare (see, for example, Vankipuram, S., Jaiswal, S., Jaiswal, M., Bajaj. A., Chandra, A., and Ojha, B. K. 2017. “Spontaneous Umbilical CSF Fistula Due to Migration of the Peritoneal End of VP [ventriculoperitoneal] Shunt: A Case Report and Review of Pathogenesis,” Journal of Pediatric Neurosciences 12(3): 285-287; Ricci, C., Velimirovic, B. M., and Fitzgerald, T. N. 2016. “Case Report of Migration of 2 Ventriculoperitoneal Shunt Catheters to the Scrotum: Use of an Inguinal Incision for Retrieval, Diagnostic Laparoscopy and Hernia Repair,” International Journal of Surgery Case Reports 29:219-222; Dolas, I., Apaydin, H. O., Yucetas, S. C., Ucar, M. D., Kilinc. S., and Ucler, N. 2016. “Cerebrospinal Fluid Leakage from the Umbilicus: Case Report and Literature Review,” International Journal of Surgery Case Reports 20:60-62).

The loss through trauma, agenesis, or resection of a lower portion of a ureter can be remedied by ureteroureteral anastomosis, or connecting the intact upper portion of the damaged ureter to its contralateral counterpart. For many shunts, such as those to compensate for an insufficiency in perfusion of muscle, or where a ureter is missing to provide a ureteroureteral crossover for access to the bladder, the intrinsic variability in volume flow-through of a shunt of the kind addressed herein is spontaneously accommodated by the expansion structure delineated above in section 2b(2)(f), entitled Expandable Structure to Accommodate Growth and Self-adjust in Size in Vascular Segment Prostheses, Lines, and Confluence Conduits Placed in Children. Attaching the tube at both ends with basic side-entry jackets should prove fully satisfactory.

This is not true, however, for pulmonary and hepatic portal shunts which must be precise to avoid the decidedly adverse consequences of undershunting, which, in the case of a transjugular extrahepatic portosystemic shunt, can lead to liver hypoxia, even hepatic infarction as well as pulmonary, biliary, and/or renal complications—an eventuality that a vascular servovalve-operated tuned shunt under the control of a fully implanted automatic disorder response system such as that addressed in this and its subsidiary sections and shown in FIG. 22F would overcome.

The need for shunt accuracy, its evaluation, and consequences continues to be elucidated (see, for example. Abdelwahed. A. H., Aboeldahb. M., and Wu, G. Y. 2024. “Effects of Transjugular Intrahepatic Portosystemic Shunt on Renal and Pulmonary Function in Hepatic Decompensation with and without Hepatorenal and Hepatopulmonary Syndromes: A Review,” Online, Journal of Clinical and Translational Hepatology (Sugar Land, Texas) 12(9): 780-791; Ferraro, A. M., Harrild, D. M., Powell, A. J., Levy, P. T., and Marx, G. R. 2024. “Evolving Role of Three-Dimensional Echocardiography for Right Ventricular Volume Analysis in Pediatric Heart Disease: Literature Review and Clinical Applications,” Journal of the American Society of Echocardiography (St. Louis, Missouri) 37(6): 634-640; Lopera Valle, J. S., Muñoz-Caicedo, B, Muñoz Duran, J. A., and Hidalgo Oviedo, J. M. 2024. “Biliary Complications Post Transjugular Intrahepatic Portosystemic Shunt in a Child with Portal Vein Cavernous Transformation,” Online, Cureus (Palo Alto, California) 16(4): e58525; Onorato, A. C. Fleishman, C., Nadorlik, H., Brown, D., Alvarado, C., and 3 others 2024. “Update of How Pediatric Cardiologists Noninvasively Evaluate Patients with Hypoplastic Left Heart Syndrome: 2013 vs. 2023,” Pediatric Cardiology (New York, New York) [in press] August 6; Yanagi, Y., Amano, M., Tamai, Y., Mizumoto, A., Nakagawa, S., and 12 others 2024. “Accuracy of Shunt Volume Measured by Three-dimensional Echocardiography and Cardiac Magnetic Resonance in Patients with an Atrial Septal Defect and a Dilated Right Ventricle,” Journal of the American Society of Echocardiography (St. Louis, Missouri) 37(8): 797-805; Mah, K. and Mertens, L. 2022. “Echocardiographic Assessment of Right Ventricular Function in Paediatric Heart Disease: A Practical Clinical Approach,” Online, CJC [Canadian Journal of Cardiology] Pediatric and Congenital Heart Disease (New York, New York) 1(3): 136-157; Kemal, H. S., Kayιkcioğlu, M., Nalbantgil, S., Can, L. H., Mogulkoc. N., and Kultursay, H. 2020. “Assessment of Right Ventricular Function in Patients with Pulmonary Arterial Hypertension-congenital Heart Disease and Repaired and Unrepaired Defects: Correlation among Speckle Tracking, Conventional Echocardiography, and Clinical Parameters,” Online, Anatolian Journal of Cardiology (Istanbul, Turkey) 23(5): 277-287; Testino, G. 2017. “Hepatorenal Syndrome: Role of the Transjugular Intrahepatic Stent Shunt in Real Life Practice,” Clujul [a county in Transylvania, Romania] Medical/Medicine and Pharmacy Reports (Cluj-Napoca, Romania) 90(4): 464-465; Knight, D. S., Grasso, A. E., Quail, M. A., Muthurangu, V., Taylor, A. M., and 4 others 2015. “Accuracy and Reproducibility of Right Ventricular Quantification in Patients with Pressure and Volume Overload Using Single-beat Three-dimensional Echocardiography,” Journal of the American Society of Echocardiography 28(3): 363-374; Medvedofsky, D., Addetia, K., Patel, A. R., Sedlmeier, A., Baumann, R., Mor-Avi. V., and Lang. R. M. 2015. “Novel Approach to Three-dimensional Echocardiographic Quantification of Right Ventricular Volumes and Function from Focused Views,” Journal of the American Society of Echocardiography (St. Louis, Missouri) 28(10): 1222-1231; Suhocki, P. V., Lungren, M. P., Kapoor, B., and Kim, C. Y. 2015. “Transjugular Intrahepatic Portosystemic Shunt Complications: Prevention and Management,” Seminars in Interventional Radiology 32(2): 123-132; He. F. L., Wang. L., Yue, Z. D., Zhao, H. W., and Liu, F. Q. 2014. “Parallel Transjugular Intrahepatic Portosystemic Shunt for Controlling Portal Hypertension Complications in Cirrhotic Patients,” Online, World Journal of Gastroenterology (Pleasanton, California) 20(33): 11835-11839; Patidar, K. R., Sydnor, M., and Sanyal, A. J. 2014. “Transjugular Intrahepatic Portosystemic Shunt,” Clinics in Liver Disease 18(4): 853-876; Renella, P., Marx, G. R., Zhou, J., Gauvreau, K., and Geva, T. 2014. “Feasibility and Reproducibility of Three-dimensional Echocardiographic Assessment of Right Ventricular Size and Function in Pediatric Patients,” Journal of the American Society of Echocardiography (St. Louis, Missouri) 27(8): 903-910; Parvinian, A., Omene, B. O., Bui, J. T., Knuttinen, M. G., Minocha, J., and Gaba, R. C. 2013. “Angiographic Patterns of Transjugular Intrahepatic Portosystemic Shunt Dysfunction and Interventional Approaches to Shunt Revision, Online, Journal of Clinical Imaging Science (Pittsford, New York) 3:19), or overshunting, which in the case of a transjugular extrahepatic portosystemic shunt can lead to esophageal varices. Such a shunt spontaneously adapts in dimensions to assure the volume flow-through responsive to the instant posture and level of activity without the need for servocontrol.

Such a shunt is ‘tuned,’ in the sense that it is response-optimized, or attuned, as controlled by an implant microcontroller programmed to take pertinent sensor inputs to continuously seek the flow-through setpoint responsive to changes in physical status, proteinaceous dietary intake, posture, level of exertion, and any other load disturbances, in which circumstance, the setpoint is moved (see, for example, National Instruments Corporation 2016. “Understanding Servo Tune,” Online at http://www.ni.com/product-documentation/2923/en/; Feng, B., Zhang, D., Mei, X., Mu, E., and Huang, X. 2015. “Investigation of the Controller Parameter Optimization for a Servomechanism,” Proceedings of the Institution of Mechanical Engineers Part B: Journal of Engineering Manufacture 229 (S1) 98-110).

It does this primarily by mechanical adjustment in through-flow, but if necessary, also commands the direct targeting of drugs to the shunt-local or remote tissue or tissues responsible for resisting return to the setpoint in accordance with a coordinated prescription-program. In most cases, these conditions and the drugs to treat them will be established and not to numerous that small drug reservoirs implanted in the pectoral region cannot respond to the various contingencies. A drug is released when the microcontroller signals the appropriate reservoir outlet pump or drip stopcock to release the prescribed dose.

2b(2)(h)(h2). The ‘Tuned’ Shunt in the Relief of Portal Venous Hypertension

A tuned shunt is unsusceptible to intimal hyperplasia, and thrombus is immediately dispelled through release of a thrombolytic through the drugline. Unlike pulmonary hypertension, wherewith the tuned shunt can divert the volume of blood directly into the superior vena cava necessary to bring the pressure within the normal range without additional processing, portal hypertension poses the problem of toxic constituents in the blood which normal liver function would metabolize to render harmless. Portal hypertension is also addressed in the section preceding this.

Transjugular intrahepatic portosystemic shunting affords the expedient of percutaneous transcatheteric placement, and alleviates the more alarming symptoms of liver impairment such as variceal bleeding, ascites, hepatic hydrothorax, testicular atrophy, gynecomastia, and thrombocytopenia, to name a few, but is subject to numerous complications both during as well as following placement, and fails to remove toxic products of digestion, drugs, and pathogens from the blood as would a normal liver. For example, in some cases, a transjugular intrahepatic portosystemic shunt will often ameliorate but not eliminate hepatic encephalopathy.

Moreover, unlike a tuned shunt, a transjugular intrahepatic portosystemic shunt is balloon adjustable in diameter upon placement but thereafter ‘dumb’ in that it is incapable of adjusting the volume of blood it allows to pass through the liver. An abnormal increase in the pressure gradient between the hepatic portal vein, its branches and the liver, portal hypertension is caused by resistance of the liver to perfusion, extrahepatic portal vein thrombosis, hemochromatosis, or primary biliary cirrhosis. Cirrhosis can both lead to or result from portal hypertension. As shown in FIG. 22F, a tuned shunt with integral pressure sensor can signal the microcontroller also integral to it in a monomorbid application or the separate master control microprocessor in a comorbid application to command the shunt servovalve to adjust the shunt flow-through cross-section.

Allowing a reduced volume of prehepatic mesenteric drainage blood to pass unobstructed without metabolic clearance through the liver as does a transjugular intrahepatic portosystemic shunt relieves the excess pressure and reduces symptoms, but does so at the expense of preserving much if not all of the injurious products of digestion and drugs in the blood arriving through the portal vein. For the treatment of liver obstructive disease, the tuned shunt is combined with an implantable hemapheresis/dialysis device referenced below in this section. The relief of elevated pressure in the portal vein with toxins allowed to bypass the liver with the expectation that the residual concentration of these will prove relatively harmless usually provides an acceptable outcome but is inherently risky.

The critical distinction between a transjugular intrahepatic portosystemic shunt and a tuned shunt combined with an implanted hemapheresis device, or implanted carrier-bound magnetically based aphereter/dialyzer as described and illustrated in FIGS. 39A and 39B of U.S. Pat. No. 11,759,186, entitled Ductus Side-entry Jackets and Prosthetic Disorder Response Systems, is that the latter provides not only hepatic portal vein decompression but makes possible the elimination of toxins, pathogens, and the risk of septicemia (see, for example, Igna, R., Girleanu, I., Cojocariu, C., Muzica, C., Huiban, L., and 13 others 2022. “The Role of Presepsin in Diagnosing Infection in Patients with Liver Cirrhosis and Overt Hepatic Encephalopathy,” Online, MDPI [Multidisciplinary Digital Publishing Institute] Diagnostics (Basel, Switzerland) 12(9): 2077; Philips, C. A. and Augustine, P. 2022. “Gut Barrier and Microbiota in Cirrhosis,” Online, Journal of Clinical and Experimental Hepatology 12(2): 625-638; Premkumar, M. and Anand, A. C. 2022. “Overview of Complications in Cirrhosis,” Online, Journal of Clinical and Experimental Hepatology 12(4): 1150-1174; Albillos, A., de Gottardi, A., and Rescigno, M. 2020. “The Gut-Liver Axis in Liver Disease: Pathophysiological Basis for Therapy,” Online, Journal of Hepatology 72(3): 558-577; Philips, C. A., Ahamed, R., Rajesh, S., George, T., Mohanan, M., and Augustine, P. 2020. “Update on Diagnosis and Management of Sepsis in Cirrhosis: Current Advances,” Online, World Journal of Hepatology 12(8): 451-474; Plano, S., Bartoletti, M., Tonon, M., Baldassarre, M., Chies, G., and 11 others 2018. “Assessment of Sepsis-3 Criteria and Quick SOFA [sequential organ failure assessment] in Patients with Cirrhosis and Bacterial Infections,” British Medical Journal. Gut 67(10): 1892-1899).

Whether the avoidance of metabolism by the liver follows placement of a transjugular intrahepatic portosystemic shunt or is the result of congenital anomalies of the portal and/or hepatic veins, the implanted aphereter/dialyzer is able to alleviate the adverse consequences of tainted mesenteric blood. As seen when the portal vein is congenitally absent, the systemic toxicity associated with the avoidance of liver clearance by outflow through congenital portosystemic shunts is a problem where secondary pulmonary hypertension, for example, is still a factor.

In some cases, closure of one or more anomalous portosystemic shunts can effect a cure for secondary pulmonary arterial hypertension (see, for example, Hlavata, T., Kaldararova, M., Klauco, F., Drangova, E., Reptova, A., and Simkova, I. 2022. “Congenital Absence of the Portal Vein as a Rare Cause of Portopulmonary Hypertension-A Case Study Series,” Online, MDPI [Multidisciplinary Digital Publishing Institute] Medicina (Kaunas, Lithuania) 58(10): 1484; (see also, Bahadori, A, Kuhlmann, B., Debray, D., Franchi-Abella, S, Wacker, J., and 3 others 2022. “Presentation of Congenital Portosystemic Shunts in Children, Online, MDPI [Multidisciplinary Digital Publishing Institute] Children (Basel, Switzerland) 9(2): 243; Xu, S., Zhang, P., Hu, L., Zhou, W., and Cheng, G. 2021. “Case Report: Clinical Features of Congenital Portosystemic Shunts in the Neonatal Period,” Online, Frontiers in Pediatrics 9:778791; Zhang, J. S. and Li, L. 2021. “Surgical Ligation of a Portosystemic Shunt for the Treatment of Type II Abernethy Malformation in 12 Children,” Journal of Vascular Surgery Venous and Lymphatic Disorders, “9(2): 444-451; Takama, Y., Nakamura, T., Santo, K., and Yoneda, A. 2020. “Liver Resection for a Congenital Intrahepatic Portosystemic Shunt in a Child with Hyperammonemia and Hypermanganesemia: A Case Report,” Online, Surgical Case Reports 6(1): 73; Franchi-Abella, S., Gonzales, E., Ackermann, O., Branchereau S., Pariente, D., and Guerin, F. 2018. “Congenital Portosystemic Shunts: Diagnosis and Treatment,” Abdominal Radiology 43(8): 2023-2036).

Following congenital portosystemic shunt vascular ligation, should an elevated level of a mesenteric blood constituent such as manganese continue to enter the systemic circulation, the objectionable substance is bonded to a superparamagnetic carrier for extraction as desired by the implanted carrier-bound magnetic aphereter (see, for example, Kakiuchi. T., Baba, Y., Minematsu, N., Doi. H., and Kumamoto, T. 2022. “Progress of Pulmonary Hypertension and High Manganese Levels through Congenital Portosystemic Shunt Closure,” Online, Clinical Case Reports 10(9): e6318). The apparatus and process are described in U.S. Pat. No. 11,759,186, entitled Ductus Side-entry Jackets and Prosthetic Disorder Response Systems. Unlike an extracorporeal apheresis machine which the patient cannot leave for up to four hours while in use, the implanted magnetic apheresis device is ambulatory, allowing the patient not only to go about his usual business but get a drink of water or go to the bathroom

Left untreated, the secondary consequences of liver impairment, specifically portal hypertension, and the serious tertiary consequences to which portal hypertension in turn gives rise can prove no less life-threatening than is the primary liver disease. When connected to the implanted aphereter/dialyzer, the tuned shunt for effecting the decompression of the portal vein shown in FIG. 22F will provide the other essential factor, to extract toxins from the bloodstream. In the peritoneal cavity, the tuned shunt conveys blood from the portal vein into the inferior vena cava.

The tertiary consequences of obstruction to passage through the liver or cirrhotic impairment include esophageal, gastric, portal vein, portopulmonary hypertension, and ectopic varices, sometimes aneurysm susceptible to rupture, thrombosis of the inferior vena cava, ascites, splenomegaly, and hepatic encephalopathy (see, for example, Amesur, N. B. and Novelli, P. 2022. “Transjugular Intrahepatic Portosystemic Shunt,” Online, Treasure Island, Florida: StatPearls Publishing Co.; Kanamalla, K. and Alwakkaa, H. 2022. “Thrombosis of the Inferior Vena Cava Secondary to Incidental Portal Vein Aneurysm,” Online, Radiology Case Reports 17(5): 1532-1535; Oliver, T. I., Sharma, B., and John, S. 2022. “Portal Hypertension,” Online, Treasure Island, Florida: StatPearls Publishing Co.; Yao, X., He., S., Wei. M., and Qin, J. P. 2022. “Influence of Different Portal Vein Branches on Hepatic Encephalopathy during Intrahepatic Portal Shunt via Jugular Vein,” Online, World Journal of Gastroenterology 28(31): 4467-4470; Thomas, C., Glinskii. V., de Jesus Perez, V., and Sahay, S. 2020. “Portopulmonary Hypertension: From Bench to Bedside,” Online, Frontiers in Medicine (Lausanne, Switzerland) 7:569413; Luo, S. H., Chu, J. G., Huang, H., Zhao, G. R., and Yao. K. C. 2019. “Targeted Puncture of Left Branch of Intrahepatic Portal Vein in Transjugular Intrahepatic Portosystemic Shunt to Reduce Hepatic Encephalopathy,” Online, World Journal of Gastroenterology 25(9): 1088-1099; Copelan, A., Kapoor, B., and Sandis, M. 2014. “Transjugular Intrahepatic Portosystemic Shunt: Indications, Contraindications, and Patient Work-up,” Online, Seminars in Interventional Radiology 31(3): 235-242; Rafiq, S. A. and Sitrin, M. D. 2007. “Portal Vein Aneurysm: Case Report and Review of the Literature,” Online, Gastroenterology and Hepatology (New York) 3(4): 296-298; Lau, H. Chew, D. K., and Belkin, M. 2002. “Extrahepatic Portal Vein Aneurysm: A Case Report and Review of the Literature Cardiovascular Surgery 10(1): 58-61).

In the chest, the tuned shunt conveys blood from the pulmonary artery into the superior vena cava. Since the nocuous substances which promote pulmonary hypertension in portal hypertension, for example, have already been extracted for expulsion from the urinary bladder by the portal tuned shunt with implanted aphereter, the need for a pulmonary tuned shunt as well to preclude the development of portopulmonary hypertension, for example, has been eliminated, as have all of the adverse sequelae associated with portal hypertension.

Barring short term extrahepatic, or nonliver, causes for a blockage in the portal vein prior to having caused significant liver impairment, the only means for dispelling the serious problems caused by liver disease currently available is to replace the liver. This can be accomplished less injuriously and with increased expectation of a successful outcome through compound vascular bypass as described above in section 2b(1)(b)(b7), entitled Metered Compound Vascular Bypass, or Switched-organ, Liver or Heart-Liver Transplantation.

The procedure is administered by a prosthetic disorder response system which also monitors the patient and directly pipe-targets immunosuppressives, antimicrobials, and anti-inflammator automatically. There is, however, no way to avoid an abdominal incision which can cause pain under humid conditions, and this makes an alternative remedy attractive. As described in section 2b(2)(h)(h1), entitled The ‘Tuned’ Shunt, as well as in this section, a tuned shunt with implanted carrier-bound magnetic hemapheresis mechanism described in U.S. Pat. No. 11,759,186, entitled Ductus Side-entry Jackets and Prosthetic Disorder Response Systems, and shown in FIGS. 39A and 39B therein, cannot be placed transcatheterically but can be placed endoscopically to provide critically superior, noxious substance-free, results.

The same device suitable for use as an implanted ambulatory kidney dialysis mechanism, FIGS. 39A and 39B in U.S. Pat. No. 11,759,186 show that through a direct connection of the inferior vena cava through a tuned shunt such as schematically shown herein in FIG. 22F positioned inferior to the bottom loop of the magnetic separation apheresis or dialysis circuit depicted in FIG. 39A, the ferrofluid and other agents would pass from the port directly into the inferior vena cava through the mainline or an accessory channel, or sideline, at a level above that of the circuit. In FIG. 39A of U.S. Pat. No. 11,759,186, the dialysate-filled flush-line, part number 79, is pressurized by the peristaltic, or roller, pump, part number 56 through the magnetic separation circuit. A more detailed description of the device appears in copending application Ser. No. 18/358,252, entitled Fully Implanted Aphereter/Dialyzers for Intracorporeal Blood Purification.

For a patient with liver disease, a drug or family of drugs clearly indicated as optimal for treating a comorbid condition but contraindicate for such a patient can promote progression of the disease to end stage and the risk of death. A transjugular intrahepatic portosystemic shunt alone would provide decompression but do nothing to remove the unmetabolized fraction of the drug from the circulation and thus make its use possible.

However, the addition to a transjugular intrahepatic portosystemic shunt of the implanted hernapheresis/hemodialysis device shown in FIGS. 39A and 39B of U.S. Pat. No. 11,759,186, entitled Ductus Side-entry Jackets and Prosthetic Disorder Response Systems, would eliminate the unmetabolized fraction of the drug to allow its use. However, the use of the device described in U.S. Pat. No. 11,759,186 in combination with a tuned shunt rather than a transjugular intrahepatic portosystemic shunt affords basic advantages in that unlike the TIPS, the tuned shunt is not limited to adjustment only upon placement, and at least one drugline makes possible the direct pipe-targeting to the shunt of an antimicrobial or anti-inflammatory, for example.

Accordingly, the combination of a tuned shunt as shown in FIG. 22F connected to the portal vein as shown in the U.S. Pat. No. 11,759,186 would dispel a liver transplant as the only option currently available to allow use of the drug or drugs indicated, to include many for patients with advanced liver failure, such as acetaminophen (paracetamol) of which the hepatotoxic potential is increased by alcohol abuse, allopurinol, amiodarone, aminoglycosides, amoxicillin, azathioprine, beta-lactam antibiotics (antibacterials), diclofenac, furosemide, isoniazid (isonicotinic hydrazide, isonicotinylhydrazide), ibuprofen, methotrexate, naproxen, phenytoin, and risperidone to name just a few.

While some drugs contraindicated for use by a patient with liver disease can be substituted, dose-adjusted, reformulated in post-liver pass form, or the problem alleviated surgically, this is not always the case (see, for example, (see, for example, Canillas, L, Pelegrina, A., Alvarez, J., Colominas-Gonzalez, E., Salar, A., and 6 others 2023. “Clinical Guideline on Perioperative Management of Patients with Advanced Chronic Liver Disease,” Online, MDPI [Multidisciplinary Digital Publishing Institute] Life (Basel, Switzerland) 13(1): 132; Bera. C. and Wong. F. 2022. “Management of Hepatorenal Syndrome in Liver Cirrhosis: A Recent Update,” Online, Therapeutic Advances in Gastroenterology 15:17562848221102679; Francis, P. and Navarro, V. J. 2022. “Drug Induced Hepatotoxicity,” Online, Treasure Island, Florida: StatPearls Publishing Co.; Jadaun, S. S. and Saigal, S. 2022. “Surgical Risk Assessment in Patients with Chronic Liver Diseases,” Online, Journal of Clinical and Experimental Hepatology 12(4): 1175-1183; Liwag. T., Wong, K., Martinez, E., and Nguyen, S. 2022. “Acute Multi-organ Toxicity during 24-hour Dosing o Intravenous Amiodarone: A Case Report,” Online, Cureus (Palo Alto, California) 14(5): e25028; Girish, C. and Sanjay, S. 2021. “Role of Immune Dysfunction in Drug Induced Liver Injury,” Online, World Journal of Hepatology 13(11): 1677-1687; Bessone, F., Hernandez, N., Mendizabal. M., Ridruejo, E., Gualano, G., and 19 others 2021. “Serious Liver Injury Induced by Nimesulide: An International Collaborative Stud,” Archives of Toxicology 95(4): 1475-1487; Zoratti. C., Moretti, R., Rebuzzi, L., Albergati, I. V., Di Somma, A., and 4 others 2021. “Antibiotics and Liver Cirrhosis: What the Physicians Need t Know,” Online, MDPI [Multidisciplinary Digital Publishing Institute] Antibiotics (Basel, Switzerland) 11(1): 31: Bassell. T., Aminlari, A. Hayden, S., Del Rosso, J., and Ly, B. T. 2020. “Phenytoin Toxicity after Transjugular Intrahepatic Portosystemic Shunt (TIPS),” Journal of Emergency Medicine 60(1): 54-57; Komolafe, O., Roberts, D., Freeman, S. C., Wilson, P., Sutton, A. J., and 9 others 2020. “Antibiotic Prophylaxis to Prevent Spontaneous Bacterial Peritonitis in People with Liver Cirrhosis: A Network Meta-analysis,” Online, Cochrane Database of Systematic Reviews 1(1): CD013125; Cheema, E., Al-Aryan, A., and Al-Hamid, A. 2019. “Medicine Use and Medicine-related Problems in Patients with Liver Cirrhosis: A Systematic Review of Quantitative and Qualitative Studies,” Online, European Journal of Clinical Pharmacology 75(8): 1047-1058; Tujios, S. R. and Lee, W. M. 2018. “Acute Liver Failure Indue by Idiosyncratic Reaction to Drugs: Challenges in Diagnosis and Therapy,” Online, Liver International 38(1): 6-14; Donati, M., Conforti, A., Lenti, M. C., Capuano, A., Bortolami, O., and 9 others 2016. “Risk of Acute and Serious Liver Injury Associated to Nimesulide and Other NSAIDs: Data from Drug-induced Liver Injury Case-control Study in Italy,” Online, British Journal of Clinical Pharmacology 82(1): 238-248; Soleimanpour, H., Safari, S., Shahsavari Nia, K., Sanaie, S. and Alavian, S. M. 2016. “Opioid Drugs in Patients with Liver Disease: A Systematic Review,” Online, Hepatitis Monthly 16(4): e32636; Fisher, K., Vuppalanchi, R., and Saxena, R. 2015. “Drug-induced Liver Injury,” Online, Archives of Pathology and Laboratory Medicine 139(7): 876-887; Bechmann, L. P., Manka, P., Best, J., Saner, F. H., Paul, A., Canbay, A., and Gerken, G. 2014. “Drug-induced Liver Injury as Predominant Cause of Acute Liver Failure in a Monocenter Study,” (in German with English abstract at Pubmed), Deutsche Medizinische Wochenschrift (Stuttgart, Germany) 139(17): 878-882; Sistanizad, M. and Peterson, G. M. 2013. “Use of Contraindicated Drugs in Patients with Chronic Liver Disease: A Therapeutic Dilemma,” International Journal of Clinical Pharmacology and Therapeutics 51(1): 1-4; Gupta, N. K. and Lewis, J. H. 2008. “Review Article: The Use of Potentially Hepatotoxic Drugs in Patients with Liver Disease,” Online, Alimentary Pharmacology and Therapeutics 28(9): 1021-1041; Westphal, J.-F. and Brogard, J.-M. 1997. “Drug Administration in Chronic Liver Disease,” Drug Safety 17(1): 47-73)

With this device, decompression is obtained by diverting sufficient blood directly from the portal vein through a servovalve inferior to the aphereter positioned along the inferior vena cava, but then unlike the resultant circumstance with a transjugular intrahepatic portosystemic shunt, objectionable constituents in the blood are extracted and delivered directly into the urinary bladder for expulsion during normal voiding, when desired, into a urinalysis sample collection cup. The implanted hemmapheresis/hemodialysis device, or aphereter/dialyzer, is compatible with the use of a transjugular intrahepatic portosystemic shunt. Connected directly to the inferior vena cava, the combination of a tuned shunt and implantable hemapheresis device completely avoid the liver.

Connection of the tuned shunt to an hepatic vein rather than directly to the inferior vena cava to create a patent path through the liver as with a transjugular intrahepatic portosystemic shunt using a tuned shunt involves intrahepatic dissection to connect the shunt to the hepatic vein. A transjugular intrahepatic portosystemic shunt avoids this but lacks the ability to monitor and respond to any change in pressure by adjustment in its flow-through cross-section or use a drugline to allow a fully implanted prosthetic disorder response system to directly pipe-target medication such as an antimicrobial to the shunt were an integral shunt sensor to signal the controller of the presence of a pathogen. The drugline must be provided with a route of exit from within the liver.

Without such a device, the toxins are passed into the pulmonary and systemic circulation where these can induce hepatorenal syndrome, and hepatopulmonary syndrome, for example, and bypassing the liver allows drugs to remain in the bloodstream that a normal liver would have eliminated (see, for example, Bassell, T., Aminlari, A. Hayden, S., Del Rosso, J., and Ly, B. T. 2020. Op cit., above in this section).

Liver and heart-liver transplantation using metered compound vascular bypass, or switched-organ transplantation addressed above in section 2b(1)(b)(b7) entitled Metered Compound Vascular Bypass, or Switched-organ, Liver or Heart-Liver Transplantation. In a simpler, or nonhierarchical context, pressure relief by a tuned shunt can be effectuated by an implanted tuned shunt with integral microcontroller and portal pressure sensor encircling or lining the portal vein, for example. If treated early, noncirrhotic portal hypertension can remain asymptomatic.

However, left untreated, extrahepatic portal vein thrombosis causes progressive liver failure with the potential to be fatal (see, for example, Samant. H., Asafo-Agyei, K. O., and Garfield, K. 2022. “Portal Vein Thrombosis,” Online, Treasure Island, Florida: StatPearls Publishing Co.; Sarquis, L. M., Trintinalha, P. O., Michaelis, W., Santos, A. L., Yokoyama, R. A., and 3 others 2022. “Non-cirrhotic Portal Vein Thrombosis—Therapeutic Challenge,” Online, Jornal Vascular Brasileiro (Salvador Bahia, Brazil) 21: e20210013; Gioia, S., Nardlelli, S., Riggio, O., Faccioli, J., and Ridola, L. 2020. “Cognitive Impairment in Non-cirrhotic Portal Hypertension: Highlights on Physiopathology, Diagnosis, and Management,” Online, MDPI [Multidisciplinary Digital Publishing Institute] Journal of Clinical Medicine (Basel, Switzerland) 11(1): 101; Li, Z., Zhang, W., Jiao, D. C., Zhou, X., Zhou, P., Si. G., and Han, X. 2021. “Clinical Study of Transjugular Intrahepatic Portosystemic Shunt Combined with AngioJet Thrombectomy for Acute Portal Vein Thrombosis in Non-cirrhosis, Online, Medicine (Baltimore, Maryland) 100(6): e24465; Gioia, S., Nardlelli, S., Ridola, L., and Riggio, O. 2020. “Causes and Management of Non-cirrhotic Portal Hypertension,” Online, Current Gastroenterology Reports 22(12): 56; Salembier, A., Verhamme, M., Verhamme, P., and Van Moerkercke, W. 2018. “Acute Non-cirrhotic Portal Vein Thrombosis: Review,” Online, Acta Gastroenterologica Belgica 81(2): 318-322; Etzion, O., Koh, C., and Heller, T. 2015. “Noncirrhotic Portal Hypertension,” Online, Clinical Liver Disease 6(3): 72-74; Sarin, S. K., Philips, C. A., and Khanna R. 2015. “Noncirrhotic Portal Hypertension: Medical and Endoscopic Management,” Online, Clinical Liver Disease 6(5): 107-111; Rajekar, H., Vasishta, R. K., Chawla, Y. K., and Dhiman, R. K. 2011. “Noncirrhotic Portal Hypertension,” Online, Journal of Clinical and Experimental Hepatology 1(2): 94-108).

Incorporating a directly pipe-targeting drugline, or accessory channel, the tuned shunt allows an optimized use of medication, medication requiring preliminary passage through the liver prepared in the post-pass form (see, for example, Sakiani, S., Heller, T., and Koh, C. 2022. “Current and Investigational Drugs in Early Clinical Development for Portal Hypertension,” Online, Frontiers in Medicine (Lausanne, Switzerland) 9:974182; Gunarathne, L. S., Rajapaksha, H., Shackel, N., Angus, P. W., and Herath, C. B. 2020. “Cirrhotic Portal Hypertension: From Pathophysiology to Novel Therapeutics,” Online World Journal of Gastroenterology 26(40): 6111-6140; Ortiz, G. A. and Garcia-Tsao, G. 2019. “Future Pharmacological Therapies of Portal Hypertension,” Online, Current Hepatology Reports 18(1): 36-48; Baiges, A., Hernandez-Gea, V., and Bosch, J. 2017. “Pharmacologic Prevention of Variceal Bleeding and Rebleeding,” Hepatology International 12 (Supplement 1): 68-80; Balducci, G., Sterpetti, A. V., and Ventura, M. 2016. “A Short History of Portal Hypertension and Its Management,” Online, Journal of Gastroenterology and Hepatology 3(3): 541-545).

Commonly seen are ascites, splenomegaly, hepatic encephalopathy, gastrointestinal bleeding and melena, kidney failure in hepatorenal syndrome, venous coagulopathy, jaundice, edema in the lower legs, caput medusa, changes in the liver, systemic, and portosystemic circulation, internal anorectal varices or hemorrhoids with bloody stool, a false sense of fullness leading to malnutrition and often sarcopenia, hypovolemic shock secondary to the rupture of varices with the risk of death, and aneurysm with or without an accumulation of clot in the portal vein.

Long accepted surgical practice, constructing a portacaval shunt-less often since the advent of the transjugular intrahepatic portosystemic shunt—to draw off enough blood from the portal vein and lead it directly into the inferior vena cava to reduce the pressure or in another procedure to draw off excess cerebrospinal fluid from the cerebral ventricles and lead it directly to the left atrium with a ventriculoatrial shunt or to the abdominal cavity by means of a lumboperitoneal shunt is anything but novel, cerebral and portacaval shunts, several valved available but far from capable of the extension to treatment of tertiary sequelae, or wide-spectrum therapy of a tuned shunt as shown schematically in FIG. 22F under hierarchical control. However, compared to earlier shunts, limited spectrum self-adjusting feedback responsive shunts provide superior medical support.

A temporary portacaval or an intraoperative portosystemic shunt may be used during conventional liver transplantation (see, for example, Crossan, K. and Jones, M. W. 2022. “Portacaval Shunt,” Online, Treasure Island, Florida: StatPearls Publishing Co.; Pietersen, L. C., Sarton, E. Alwayn, I., Lam, H. D., Putter, H., van Hoek, B., and Braat. A. E. 2019. “Impact of Temporary Portocaval Shunting and Initial Arterial Reperfusion in Orthotopic Liver Transplantation,” Liver Transplantation 25(11): 1690-1699; Nacif. L. S., Zanini, L. Y., Sartori, V. F., Kim, V. Rocha-Santos, V., Andraus, W., and Carneiro d' Albuquerque, L. 2018. “Intraoperative Surgical Portosystemic Sunt in Liver Transplantation: Systemic Review and Meta-analysis,” Online, Annals of Transplantation 23:7211-732: Rayar, M., Levi Sandri, G. B., Cusumano, C., Locher, C., Houssel-Debry, P., and 7 others 2017. “Benefits of Temporary Portocaval Shunt during Orthotopic Liver Transplantation with Vena Cava Preservation: A Propensity Score Analysis,” Online, Liver Transplantation 23(2): 174-183; Davila, D., Bartlett, A., and Heaton, N. 2008. “Temporary Portocaval Shunt in Orthotopic Liver Transplantation: Need for a Standardized Approach?,” Online, Liver Transplantation 14(10): 1414-1419; Rosemurgy, A. S., Bloomston, M., Clark, W. C., Thometz, D. P., and Zervos, E. E. 2005. “H-graft [novel vascular anastomotic scheme] Portacaval Shunts versus TIPS [transjugular intrahepatic portosystemic shunt],” Online, Annals of Surgery (Philadelphia, Pennsylvania) 241(2): 238-246).

Although the transjugular intrahepatic portosystemic shunt has to a significant extent supplanted the portocaval shunt, it is evident that a wider symptom detection and response system with sensors disseminated to relevant sites that ‘lies in wait’ to detect and respond to secondary and tertiary adverse sequelae is essential to improve its dependability, especially following liver transplantation (see, for example, Karagiannakis, D. S. and Stefanaki, K. 2023. “Spleen Stiffness: A Predictive Factor of Dismal Prognosis in Liver Cirrhosis,” Online, Clinical Journal of Gastroenterology (Tokyo, Japan) 16(2): 121-129; Yao, Y., Satapathy, S. K., Fenandes, E. S. M., Ramirez-Fernandez, O., Vitale, A., and Chen, Z. 2022. “Hepatic Venous Pressure Gradient (HVPG) Predicts Liver Failure after Transjugular Intrahepatic Portosystemic Shunt: A Retrospective Cohort Study,” Online, Annals of Translational Medicine 10(20): 1122; Rajesh, S., George, T., Phillips, C. A. Ahamed. R., Kumbar, S., and 3 others 2020. “Transjugular Intrahepatic Portosystemic Shunt in Cirrhosis: An Exhaustive Critical Update,” Online, World Journal of Gastroenterology 26(37): 5561-5596; Schindler, P., Heinzow, H., Trebicka, J., and Wildgruber, M. 2020. “Shunt-induced Hepatic Encephalopathy in TIPS: Current Approaches and Clinical Challenges,” Online, MDPI [Multidisciplinary Digital Publishing Institute] Journal of Clinical Medicine (Basel, Switzerland) 9(11): 3784; Zhu, H., Guo, H., Yin, X., Yang, J., Yin, Q., and 5 others 2020. “Spleen Stiffness Predicts Survival after Transjugular Intrahepatic Portosystemic Shunt in Cirrhotic Patients,” Online, BioMed Research International (New York, New York) 2020:3860390; Li, Y. and Pang, H. 2019. “A Model to Predict Early Hepatic Encephalopathy in Patients Undergoing Transjugular Intrahepatic Portosystemic Shunt,” Online, Turkish Journal of Gastroenterology 30(8): 702-707; De Santis, A., Nardelli, S., Bassanelli, C., Lupo, M., Iegri, C., and 4 others 2018. “Modification of Splenic Stiffness on Acoustic Radiation Force Impulse Parallels the Variation of Portal Pressure Induced by Transjugular Intrahepatic Portosystemic Shunt,” Journal of Gastroenterology and Hepatology 33(3): 704-709; Li, H. B., Yue, Z. D., Zhao, H. W., Wang, L, Fan, Z. H., and 3 others 2018. “Pathological Features of Mitochondrial Ultrastructure Predict Susceptibility to Post-TIPS Hepatic Encephalopathy,” Online, Canadian Journal of Gastroenterology and Hepatology 2018:4671590; Nardelli, S., Giola, S., Ridola, L., and Riggio, O. 2018. “Radiological Intervention for Shunt Related Encephalopathy,” Journal of Clinical and Experimental Hepatology 8(4): 452-459).

Such a basic shunt is usually placed to bypass the obstruction to flow. If the native blood supply is obstructed entirely into the parenchyma or deeper tissue, the distal side-entry jacket is replaced with a nonjacketing side-entry connector as described in U.S. Pat. No. 11,013,858, entitled Nonjacketing Side-entry Connectors and Prosthetic Disorder Response Systems, wherein FIGS. 17-19 show attachment to reach deep tissue through an adaptation of the Vineberg procedure. However, where quantifiable precision is essential, an extrahepatic servovalve shunt in place of an intrahepatic transjugular portosystemic shunt, for example, can ‘walk a fine line,’ that is, continuously adjust to the ideal flow-through so that neither undershunting with inadequate perfusion, or overshunting, with variceal hemorrhaging, primarily esophageal, ascites, and likely hepatic encephalopathy.

Unlike the type shunt set forth herein, a transjugular intrahepatic portosystemic shunt is an inflexible metal, silicone, or polypropylene stent, and despite the lack of direct drug delivery pipeline, may be thrombophilic, prompting the use of a systemically dispersed antithrombotic. Such a stent shunt does not incorporate a one-way pressure-relief valve with ancillary shunt leading into a large neighboring vessel. It is incapable of adapting to changes in the flow-rate or allowing the direct delivery of medication. To isolate the extrahepatic shunt from injury and reduce the risk of biliary obstruction (Meng. J., Yang, S., Ding. W., He. C., and Wu, X. 2016. “Biliary Obstruction following Transjugular Intrahepatic Portosystemic Shunt Creation in Patients with Variceal Bleeding,” Journal of Vascular and Interventional Radiology 27(4): 558-561), it is better positioned away from the anterior body wall.

In fundamental contrast, a ‘tuned’ shunt is more capable than those on the market by virtue of its compatibility with control by an arm of a hierarchical support system to not just automatically adjust the volume of blood flowing through the portal vein but by automatically delivering medication into it as needed. The prescription-program of such a system can be expanded to detect and treat the secondary and tertiary sequelae of the primary liver disease. Following replacement of the diseased liver, the prosthetic backup system is reprogrammed accordingly. Tuned shunts to serve as improved replacements for surgically constructed shunts such as a portacaval to move blood from the portal vein directly into the inferior vena cava or another large neighboring vessel are addressed in the preceding section, 2b(2)(h)(h1), entitled The ‘Tuned’ Shunt.

Some currently marketed portacaval, splenorenal, and mesocaval-applicable hepatic as well as cerebral ventricle applicable shunts are controllable to self-adjust to changes in through-pass volume or pressure; however, these employ components which cannot be implanted in an ambulatory automatic prosthetic disorder response system. The mechanisms involved are incompatible with control and disseminated support by an implanted system, and directed to the control of drainage and primary hypertension, these may significantly reduce but cannot detect and treat any secondary or tertiary sequelae as might eventually arise (see, for example, Garg. K., Gupta, D., Singh, M., Chandra, P. S., and Kale, S. S. 2022. “Comparison of a Bolt-connected External Ventricular Drain with a Tunneled External Ventricular Drain-A Narrative Review and Meta-analysis,” Neurosurgical Review 45(2): 937-949; Pandit, A. S., Palasz, J., Nachev. P., and Toma, A. K., 2022. “Mechanical Complications of External Ventricular and Lumbar Drains,” World Neurosurgery 166: e140-e154; Arts. S., van Bilsen, M., van Lindert, E. J., Bartels, R. H., Aquarius, R., and Boogaarts, H. D. 2021. “Implementation of an Automated Cerebrospinal Fluid Drainage System for Early Mobilization in Neurosurgical Patients,” Online, Brain Sciences 11(6): 683; Scheffler, P., Oertel, M. F., and Stieglitz, L. N. 2021. “Comparison between Flow-regulated and Gravitational Shunt Valves in the Treatment of Normal Pressure Hydrocephalus: Flow-Grav Study,” Neurosurgery 89(3): 413-419; Desai, V. R., Sadrameli, S. S., Jenson, A. V., Asante, S. K., Daniels B., Trask. T. W. and Britz, G. H. 2020. Op cit. above in section 2b(2)(h)(h1), entitled The ‘Tuned’ Shunt; Assad, S. K. and Bjarkam, C. R. 2019. “The Aalborg Bolt-connected Drain (AFCD) Study: A Prospective Comparison of Tunneled and Bolt-connected External Ventricular Drains,” Acta Neurochirurgica (Vienna, Austria) 161(1): 33-39; Roach, J., Gaastra, B., Bulters, D., and Shtaya, A. 2019. “Safety, Accuracy, and Cost Effectiveness of Bedside Bolt External Ventricular Drains (EVDs) in Comparison with Tunneled EVDs Inserted in Theaters,” World Neurosurgery 125: e473-e478; Linsler, S., Schmidtke, M., Steudel, W. I., Kiefer, M., and Oertel, J. 2013. “Automated Intracranial Pressure-controlled Cerebrospinal Fluid External Drainage with LiquoGuard,” Acta Neurochirurgica (Vienna, Austria) 155(8): 1589-1595). If and when such mechanisms will completely eliminate the primary hypertension, a need to deal with disseminated secondary and tertiary consequences would cease to exist.

As it is, to achieve such a wider if not comprehensive control than a shunt can provide demands a hierarchical control system as described in U.S. Pat. No. 11,759,186 entitled Ductus Side-entry Jackets and Prosthetic Disorder Response Systems and Ser. No. 17/689,880, entitled Prosthetic Disorder Control Systems. What is novel is the ability to provide an hepatic or cerebral shunt through which not only can an implanted prosthetic disorder response system continuously and automatically respond to sensors positioned strategically to adjust the rate of blood to be drawn off to maintain a pressure so that it falls within the normal range but which also monitor the sites prone to the tertiary sequelae associated with the primary condition.

Unlike liver dialysis, which remains unable to provide the entire function of a healthy liver and serves as a temporary expedient to retard the progression of tertiary disease pending a liver transplant, if shunt control is integrated as one arm in a hierarchical control system to continuously monitor and respond to deviations at the sites of tertiary injury, the shunt can indeed achieve the status of a cure. This assumes that the sites of secondary and tertiary disease enumerated above are each provided with the sensors and directly pipe-targeting jackets or connectors essential to detect the need for and delivery the drugs appropriate for each site.

Such assumes that notwithstanding the ability of the automatic system to correct deviations in pressure over time, an accumulation of deviations in the context of refractory disease could eventually succeed in producing the disseminated tertiary injury to which such intermittent hypertension would give rise. A cure properly connotes a relieving of the pathology to the end of life, and to approximate such a result in lieu of a liver transplant would supplant a procedure that could easily result in the loss of that life prematurely. With a transjugular intrahepatic portosystemic shunt combined with liver dialysis to eliminate residual waste products and a beta blocker, the progression of these consequences can be forestalled.

Until a tuned shunt under perfected hierarchical control is developed, the definitive treatment where such symptoms exist will remain a liver transplant. A ‘tuned’ portacaval shunt treats not only the primary and secondary symptoms but the data obtained from its performance is used by the hierarchical control system to disseminate therapy to these remote sites in order to achieve a homeostasis optimized under the circumstances.

Using a drugline inserted into tan accessory channel of the inlet side-entry jacket, valve, or servovalve, the most common causes cited for eventual failure of surgically constructed shunts, obstruction and infection, can be counteracted through the automatic detection and directly pipe-targeted delivery of a solvent or antimicrobial, and an accumulation of clot can be counteracted through the automatic detection and directly targeted pipe-delivery of an antithrombotic. The rate and volume of flow through a surgically created shunt is usually determined by the upstream flow, so that the causative factor should always be medically addressed. The ability to directly target medicinals into the shunt can, however, represent a significant advantage. Local reduction of portal hypertension is addressed above in section 2b(2)(h)(h2), entitled The ‘Tuned’ Shunt in the Relief of Portal Venous Hypertension.

Postoperative complications of conventional shunts are common, with revision surgery posing much risk (see, for example, Schindler, P., Heinzow, H., Trebicka, J., and Wildgruber, M. 2020. “Shunt-induced Hepatic Encephalopathy in TIPS [transjugular intrahepatic portosystemic shunt]: Current Approaches and Clinical Challenges,” Online, MDPI [Multidisciplinary Digital Publishing Institute] Journal of Clinical Medicine (Basel, Switzerland) 9(11): 3784; Jeng. E. I., Voskresensky, I., Beck, A W., and Lee, T. C. 2017. “Femorofemoral Shunt during Surgery for Acute DeBakey Type 1 Dissection to Treat Peripheral Extremity Malperfusion,” Online, Annals of Thoracic Surgery (Amsterdam, North Holland) 103(1): e17-e20; Kobayashi, L. M., Costantini, T. W., Hamel, M. G., Dierksheide, J. E., and Coimbra, R. 2016. “Abdominal Vascular Trauma,” Online, (London, England) 1(1): e000015; Perry, B. C. and Kwan, S. W. 2015. “Portosystemic Shunts: Stable Utilization and Improved Outcomes, Two Decades after the Transjugular Intrahepatic Portosystemic Shunt,” Online, Journal of the American College of Radiology (New York, New York) 12(12 Pt B): 1427-33.).

Secondary to end-stage liver disease or toxicity, portal hypertension and its consequences are briefly addressed above in section 2b(1)(b)(b7), entitled Metered Compound Vascular Bypass, or Switched-organ Liver or Heart-Liver Transplantation. There it is stressed that to shunt off the correct amount of blood to bring the pressure within the normal range in the portal vein, which if not reduced would lead to numerous disseminated sequelae, is anything but original; it is the instituting an implanted automatic response system to continuously or intermittently gauge the pressure and control outflow through the shunt that is novel.

Extrahepatic, such an hepatic portal vein shunt is not limited to outlet into the right hepatic vein, but can drain directly into the inferior vena cava or another larger vein. Then, if the right hepatic vein is free of disease, having been bypassed, it continues to drain normally without any dissection or stenotic response, which is, however, medically reversed. If, however, the right hepatic vein is diseased or constricted, some dissection notwithstanding, the shunt outlet servovalve is connected to it in order to allow use of the valve accessory channel or channels to directly target remedial drugs and a thrombolytic into the vein.

Just as all forms of vascular hypertension, portal hypertension, or resistance to free flow through the hepatic portal vein, is caused by a lack of portal venous blood uptake within the end organ, that is, intrahepatic, usually due to cirrhosis; and/or obstruction within the hepatic portal venous system, usually by clot secondary to gastrointestinal malignancy, primarily, of the liver and/or pancreas, or prehepatic; and/or between the hepatic vein and inferior vena cava, or posthepatic. The relief of portal pressure in end-stage liver disease is usually an interim means of palliation pending the availability of a transplant liver, the application thereto of the switch approach addressed herein.

Addressed above in section 2b(1)(b)(b7), entitled Metered Compound Vascular Bypass, or Switched-organ Liver or Heart-Liver Transplantation as well, when the liver is diseased, usually and especially when cirrhotic, the adverse sequelae of portal hypertension and liver dysfunction are far-reaching, to include further injury even to the heart (Huang, W. A., Dunipace, E. A., Sorg, J. M., and Vaseghi, M. 2018. “Liver Disease as a Predictor of New-onset Atrial Fibrillation,” Journal of the American Heart Association (Oxford, England) 7(15): e008703; Melchio, R., Margaria, F., Bracco, C., Pomero, F., and Fenoglio, L. M. 2016. “Respiratory Failure in Portal Hypertension: At the Heart of the Matter,” Internal and Emergency Medicine 11(5): 721-722; Wroński, J., Fiedor, P., Kwolczak, M., and Gomnicka, B. 2015. “Retrospective Analysis of Liver Cirrhosis Influence on Heart Walls Thickness,” Pathology, Research, and Practice 211(2): 145-149; Moller, S., Mortensen, C., Bendtsen, F., Jensen, L. T., Gotze, J. P., and Madsen, J. L. 2012. “Cardiac Sympathetic Imaging with mIBG [quantified (123) 1-metaiodobenzylguanidine (mIBG) scintigraphy] in Cirrhosis and Portal Hypertension: Relation to Autonomic and Cardiac Function,” American Journal of Physiology. Gastrointestinal and Liver Physiology 303(11): G1228-G1235) and considerably disrupting digestion (Kalaitzakis E. 2014. “Gastrointestinal Dysfunction in Liver Cirrhosis,” World Journal of Gastroenterology 20(40): 14686-14695; Kalaitzakis, E., Sadik, R., Holst, J. J., Ohman, L., and Bjornsson, E. 2009. “Gut Transit is Associated with Gastrointestinal Symptoms and Gut Hormone Profile in Patients with Cirrhosis,” Clinical Gastroenterology and Hepatology 7(3): 346-352).

Nonadjustable hepatic portal venous pressure relief through transjugular intrahepatic portosystemic shunting has been in use for three decades, and while having demonstrated considerable relief from the complications of portal hypertension, such as esophageal variceal bleeding and ascites, it is still susceptible to numerous adverse sequelae, to include hemorrhage, liver failure, accumulation of clot, and hepatic encephalopathy (see, for example, Rabei, R., Mathevosian, S., Tasse, J., Madassery, S., Arslan, B., Turba, U., and Ahmed, O. 2018, Op cit.; Zhao, H., Liu, F., Yue, Z., Wang, L., Fan, Z., and He, F. 2017. “Clinical Efficacy of Transjugular Intrahepatic Portosystemic Shunt in the Treatment of Hepatopulmonary Syndrome,” Medicine (Baltimore, Maryland) 96(49): e9080; Suhocki, P. V., Pereira, K., Carrion, A. F., Salsamendi, J., Doshi, M., Baker, R., and Kably, I. 2016. “Endovascular Management of Refractory Hepatic Encephalopathy Complication of Trans jugular Intrahepatic Portosystemic Shunt (TIPS): Comprehensive Review and Clinical Practice Algorithm,” Cardiovascular and Interventional Radiology 39(2): 170-182; He, F., Dai, S., Xiao, Z., Wang, L., Yue, Z., and 5 others 2016. “Pathological Predictors of Shunt Stenosis and Hepatic Encephalopathy after Transjugular Intrahepatic Portosystemic Shunt,” Online, BioMed Research International 2016:3681731; Lungren, M. P., Kapoor, B., and Kim, C. Y. 2015. “Transjugular Intrahepatic Portosystemic Shunt Complications: Prevention and Management,” Seminars in Interventional Radiology 32(2): 123-132; Parvinian, A., Omene, B. O., Bui, J. T., Knuttinen, M. G., Minocha, J., and Gaba, R. C. 2013. “Angiographic Patterns of Transjugular Intrahepatic Portosystemic Shunt Dysfunction and Interventional Approaches to Shunt Revision,” Journal of Clinical Imaging Science 3:19; Leong, S., Kok, H. K., Govender, P., and Torreggiani, W. 2013. “Reducing Risk of Transjugular Intrahepatic Portosystemic Shunt Using Ultrasound Guided Single Needle Pass,” World J Gastroenterology 19(22): 3528-3530; Liu, K., Fan, X. X., Wang, X. L., He, C. S., and Wu, X. J. 2012. “Delayed Liver Laceration following Transjugular Intrahepatic Portosystemic Shunt for Portal Hypertension,” World J Gastroenterology 18(48): 7405-7408; additional references pertinent to the hepatopulmonary syndrome are cited above in section 2b(1)(b)(b7), entitled Metered Compound Vascular Bypass, or Switched-organ Liver or Heart-Liver Transplantation).

Another strategy for relieving pressure in the portal vein is to surgically divert nonintestinal inflow into the portal vein to another vein for return to the systemic circulation by shunting, thus circumventing and reducing flow through the portal vein (see, for example, Jain, S., Kalla, M., Suleman, A., and Verma, A. 2017. “Proximal Spleno-renal Shunt with Retro-aortic Left Renal Vein in a Patient with Extra-hepatic Portal Vein Obstruction: First Case Report,” Online, BioMed Central Surgery 17(1): 65; Mishra, P. K., Patil, N. S., Saluja, S., Narang, P., Solanki, N., Varshney, V. 2016. “High Patency of Proximal Splenorenal Shunt: A Myth of Reality?—A Prospective Cohort Study,” International Journal of Surgery 27:82-87; Rezende-Neto, J. B., Petroianu, A., and Santana, S. K. 2008. “Subtotal Splenectomy and Central Splenorenal Shunt for Treatment of Bleeding from Roux en Y Jejunal Loop Varices Secondary to Portal Hypertension,” Digestive Diseases and Sciences 53(2): 539-543; Shah, O. J. and Robbani, 1. 2005. “A Simplified Technique of Performing Splenorenal Shunt (Omar's Technique),” Texas Heart Institute Journal 32(4): 549-554; Petroianu, A. 1988. “Treatment of Portal Hypertension by Subtotal Splenectomy and Central Splenorenal Shunt,” Postgraduate Medical Journal 64(747): 38-41; Linton R. R., Jones, C. M., and Volwiler W. 1947. “Portal Hypertension; the Treatment by Splenectomy and Splenorenal Anastomosis with Preservation of the Kidney,” Surgical Clinics of North America 27:1162-1170).

Other approaches seek to bypass the portal vein by redirecting drainage from viscera normally draining into the portal vein. These include the splenorenal, or Warren, shunt, which involves surgical anastomosis of the distal splenic vein to the left renal vein, thus preventing drainage from the stomach, spleen, and pancreas into the portal vein (see, for example, Gu, S., Chang, S., Chu, J., Xu, M., Yan, Z., Liu, D. C., and Chen, Q. 2012. “Spleno-adrenal Shunt: A Novel Alternative for Portosystemic Decompression in Children with Portal Vein Cavernous Transformation,” Journal of Pediatric Surgery 47(12): 2189-2193).

Much the same criticism can be made of surgical shunting in general to include transjugular intrahepatic portosystemic shunting, even when furnished with a kind of shunt compressing choke, referred to as ‘constrained,’ wherewith the shunt is encircled by a balloon that can be used to compress the shunt in a follow-up interventional procedure (Perry, B. C., Monroe, E. J., and Shivaram, G. 2018. “Adjustable Diameter TIPS in the Pediatric Patient: The Constrained Technique,” Diagnostic and Interventional Radiology 24(3): 166-168; Rabei, R., Mathevosian, S., Tasse, J., Madassery, S., Arslan, B., Turba, U., and Ahmed, O. 2018. “Primary Constrained TIPS for Treating Refractory Ascites or Variceal Bleeding Secondary to Hepatic Cirrhosis,” British Journal of Radiology 91(1083): 20170409; Farsad, K., Kolbeck, K. J., Keller, F. S., Barton, R. E., and Kaufman, J. A. 2015. “Primary Creation of an Externally Constrained TIPS: A Technique to Control Reduction of the Portosystemic Gradient,” American Journal of Roentgenology 204(4): 868-871).

In contrast to a shunt using continuously variable servovalves controllable automatically by the implanted control system inside the body and before implantation from outside the body, placing a transjugular intrahepatic portosystemic shunt is especially demanding of surgical skill. Even simplified (Shah, O. J. and Robbani, I. 2005, Op cit.), such techniques, especially in a neonate may involve intricate dissection, risk problem bleeding, and produce a shunt inaccessible once the patient is closed. When placed in a small child, even with a constrained shunt, an eventual need for reentry to open the shunt to accommodate growth is a certainty.

As indicated, a self-expanding stent-graft used as a transjugular intrahepatic portosystemic shunt may change in gauge out of step with growth of an infant; within the first six weeks following placement, such a stent expands to far too large a diameter (Pieper, C. C., Jansen, C., Meyer, C., Nadal, J., Lehmann, J., and 3 others 2017. “Prospective Evaluation of Passive Expansion of Partially Dilated Transjugular Intrahepatic Portosystemic Shunt Stent-grafts—A Three-dimensional Sonography Study,” Journal of Vascular and Interventional Radiology 28(1): 117-125; Pieper, C. C., Sprinkart, A. M., Nadal, J., Hippe, V., Meyer, C., Schild, H. H., and Thomas, D. 2015. “Postinterventional Passive Expansion of Partially Dilated Transjugular Intrahepatic Portosystemic Shunt Stents,” Journal of Vascular and Interventional Radiology 26(3): 388-394).

In contrast, shunting with inlet and outlet vascular servovalve-jackets provides continuously variable volume of flow through the shunt as well as the direct release of drugs through either jacket, and where the condition is variable, an implant sensor-fed microcontroller can instantly adjust the continuously variable servovalve aperture as well as directly target drugs from implant drug reservoirs in accordance with the controller prescription-program.

Such shunting affords an alternative to endoscopic third ventriculostomy for patients in whom the risks for basilar artery rupture, injury to the hypothalamus-pituitary complex, or the fomix, for example, or for postoperative occlusion or leaks are high. In this connection, the usual deterrent to the introduction of placing an implant—the risks of infection or obstruction—are eliminated by the availability of the accessory channel or channels in the inlet servovalve to release antimicrobials or obstruction breakdown agents directly into the cerebrospinal fluid shunt.

Differential fixed pressure shunting has also been applied to the concurrent drainage of an extracerebral collection of fluid while managing the hydrocephalus (see, for example, Craig, D., Winston, K. R., Folzenlogen, Z., and Beauchamp, K. A. 2018. “Differential Pressure Shunt for Simultaneous Diversion of Ventricular Fluid and Extracerebral Fluid,” Pediatric Neurosurgery 53(1): 13-17). Blood substantially incompressible and the heart as a pulsatile incremental driver remote from the shunt, reduction in the intensity of flow through such a constrained shunt may be attributed to the reduction in amplitude, or damping, of the driving impulses by interference with continued flow about the circumference of each diastolic pulse as it enters the shunt (Applied Flow Technology Corporation 2010. Modeling Choked Flow Through an Orifice, available Online at https://docplayer.net/40297743-Modeling-choked-flow-through-an-orifice.html and https://www.aft.com/documents/AFT-Modeling-Choked-Flow-Through-Orifice.pdf; International Organization for Standardization 2013. “Measurement of Fluid Flow by Means of Pressure Differential Devices Inserted in Circular Cross-section Conduits Running Full,” [Standard ISO 5167] available Online at https://www.iso.org/standard/28064.html, https://www.iso.org/standard/30190.html; Engineering Department, Crane Plumbing Company 1988, Flow of Fluids Through Valves, Fittings and Pipe, New York, New York: Crane Plumbing Company, available Online at https://engkarrar.files.wordpress.com/2013/01/flow-of-fluids-through-valve-fittings-and-pipes.pdf).

While the mechanism is different, the type valves addressed below in section 2b(2)(i), entitled Vascular Servochokes, and the subsections thereto, are also capable of reducing the pressure and increasing the velocity antegrade or downstream.to its position. For this application, either extravascular servo chokevalve or intravascular side-entry diversion servovalve-jackets can be placed at either end of the shunt made of conventional shunt tubing, and the choke servovalve can also be elongated to constitute the entire shunt line.

In any of these extrahepatic servotuned shunts, the choke servovalves or side-entry diversion jacket servovalves are able to compensate for pulse rate and damping upon shunt entry by moving to the new setpoints commanded by the implanted microcontroller, or in a hierarchical control system of which the liver microcontroller represents but one arm of control, the implanted master microprocessor. Where the servovalves are positioned at either end of conventional shunt tubing, the three elements of the shunt—the valves at either end and tubing are treated as a unit based upon sensor hemodynamic input to the controller.

Adaptable to the instant physiological, that is exertional, emotional, postural, and chemical to include metabolic state, the automatically and continuously adjusted choke servovalve is critically superior to a conventional constrained shunt: where the latter requires reentry to adjust to a new fixed setting, the servochoke responds immediately to the need to adjust the pass-through for the instant load. Moreover, the choke servovalve incorporates one or more accessory channels that allow the direct targeting to the shunt of drugs. The scheme for controlling an end-actuator, or end-effector, such as a choke or diversion servovalve, is described and illustrated in U.S. Pat. No. 11,389,171, entitled Integrated System for the Infixion and Retrieval of Implants, and in U.S. Pat. No. 11,759,186, entitled Ductus Side-entry Jackets and Prosthetic Disorder Response Systems. The same concept had been applied earlier in the latter application to prosthetic sphincters, esophageal assist devices, and prosthetic esophagi.

Associated with liver failure and portal hypertension, in a patient with hepatic encephalopathy relatively mild as not to require hospitalization but undependable for prescription compliance is well served through the automatic and immediate targeting of a lactitol, rifaximin, L-omithine, and L-aspartate ‘cocktail’ directly into the gut (see, for example, Wang, P., Zhang, Y. J., Li, Y. R., Xia, X. Y., and Lv, S. Y. 2018. “STORE-gastrointestinal Functions and Gastrointestinal Hormones in Patients with Liver Failure,” Medicine (Baltimore, Maryland) 97(48): e13167; Kalaitzakis E. 2014, Op cit.; Gunnarsdottir, S. A., Olsson, R., Olafsson, S., Cariglia, N., Westin, J., Thiodleifsson, B., and Bjornsson, E. 2009. “Liver Cirrhosis in Iceland and Sweden: Incidence, Aetiology and Outcomes,” Scandinavian Journal of Gastroenterology 44(8): 984-993).

Controlled by a suitable prescription-program responsive to implanted sensors that continuously indicate the indicia appurtenant of portal venous flow, a side-entry servovalve jacket positioned about the portal vein as the entry into a shunt can allow the dynamic and immediate adjustment in the volume of flow through the shunt automatically and make it possible for the pressure to be adjusted from outside the body. Given the way the servovalve-jacket is placed along the substrate vessel, less dependency upon operator skill is involved, so that hemorrhage should be eliminated.

External constraint reduces the malperfusion of the liver from overshunting, with the loss to the liver of nutrients, oxygen, and leukocyte protection, is able to decrease hepatic encephalopathy, and can be adjusted at a later date. However, adjusting such a shunt requires a follow-up transluminal procedure, which is anything but immediately responsive to the instant need. Such a shunt does not respond to means that continuously sense, initiate, and confirm the optimal shunt flow-through to balance the maximum reduction in hypertension consistent with minimizing the adverse effects of the hypertension, much less automatically from outside the body, or with the automatic cooperative release of medication.

The ability to adjust the volume of blood channeled through a ductus side-entry servovalve-jacketed shunt to no more than compensate for the pressure relief shortfall obtained with drugs and to direct the targeting of drugs into the ductus side-entry servovalve-jackets at the proximal, or portal venous and hepatic attachments should minimize the complications of conventional shunting, which incapable of immediate response to the instant need automatically, may be described as ‘dumb.’

A means of shunting that allows continuous variability in the volume of blood that passes through the shunt, and more specifically, dynamically adjusts the volume of flow-through to the minimum based upon implant sensor input, is able to minimize these negative factors. A shunt using an inlet or both inlet and outlet servovalves can take the place of any existing shunt and critically adds to the utility of these the ability to dynamically adjust flow-through to the least volume in response to the immediate need automatically, as well as target medication directly into the shunt. Significantly, such a shunt allows the automatic reduction to the least injurious volume of blood bypassed to reduce hypertension while minimizing life threatening side effects.

While extrahepatic and requiring access through small incisions to place endoscopically, like the distal splenorenal and similar shunts, rather than transluminally, like the transjugular intrahepatic portosystemic shunt, the use of a ductus side-entry servovalve as the entry jacket of a portal hypertension relief shunt offers advantages over transjugular intrahepatic portosystemic shunting from the left branch of the portal vein to the hepatic vein or any other position. These include the direct piping to the shunt of antimicrobials, anticoagulants, thromobolytics (fibrinolytics, ‘clot-busting,’ or any other drug administered as a fluid, and free adjustability in shunting volume from outside the body, such as to ameliorate the particularly troublesome problem of hepatic encephalopathy, for example.

The addition to transjugular intrahepatic portosystemic shunt of directly targeted release of medication (specify drugs used) coordinated with pressure relief through diversion of that least fraction of the portal venous flow-through essential to compensate for the shortfall in pressure relief afforded by medication alone reduces the adverse sequelae encountered following the use of conventional components. Diverting excess pressure has precedence in transjugular intrahepatic portosystemic shunting, where the volume of diverted blood is not instantly and continuously variable from outside the body through radio remote control of the diversion servovalve.

Portal hypertension with splanchnic vasodilatation, varices, and variceal bleeding secondary to cirrhosis resulting in morbidity and mortality is not necessarily dispelled following a conventional liver transplant, so that the elimination of the interruption in the flow of blood, the associated ischemia/reperfusion injury, clamping, dissection trauma, and general anesthesia provided by the compound vascular bypass technique should almost certainly yield better and relatively complication-free outcomes.

Another consideration is that—as is true of any solid organ transplant—the considerable reduction in trauma and stress of the bypass technique should qualify patients who otherwise would not qualify for a liver (or any other solid organ) transplant as well as yield much improved outcomes for every category of patient (see, for example, Al Alawi, A. M., Al Kaabi, H., Al Falahi, Z., Al-Naamani, Z., and Al Busafi, S. 2024. “Machine Learning-powered 28-day Mortality Prediction Model for Hospitalized Patients with Acute Decompensation of Liver Cirrhosis,” Online, Oman Medical Journal (Muscat, Oman) 39(3): e632; Zhai, Y., Hai, D., Zeng, L., Lin, C., Tan, X., and 7 others 2024. “Artificial Intelligence-based Evaluation of Prognosis in Cirrhosis,” Online, [BioMed Central] Journal of Translational Medicine (London, England) 22(1): 933; Al Kaabi, H., Al Alawi, A. M., Al Falahi, Z., Al-Naamani, Z., and Al Busafi, S. A. 2023. “Clinical Characteristics, Etiology, and Prognostic Scores in Patients with Acute Decompensated Liver Cirrhosis,” Online, MDPI [Multidisciplinary Digital Publishing Institute] Journal of Clinical Medicine (Basel, Switzerland) 12(17): 5756; Daglilar, E., Connolly, S. E., Tahan, V., Cohen, A., and Therapondos, G. 2020. “The Utilization and Diagnostic Yield of Upper Endoscopy for Evaluation of Gastrointestinal Symptoms within the First Year After Liver Transplantation,” Online, Cureus (Palo Alto, California) 12(11): e11323; Gunarathne, L. S., Rajapaksha, H., Shackel, N., Angus, P. W., and Herath, C. B. 2020. “Cirrhotic Portal Hypertension: From Pathophysiology to Novel Therapeutics,” Online, World Journal of Gastroenterology (Pleasanton, California) 26(40): 6111-6140; Mahmud, N. 2020. “Selection for Liver Transplantation: Indications and Evaluation,” Online, Current Hepatology Reports (New York, New York) 19(3): 203-212; Unger, L., Mandorfer, M., and Reiberger, T. 2019. “Portal Hypertension after Liver Transplantation-Causes and Management, Online, Current Hepatology Reports (New York, New York) 18(3): 1-8; Korda, D., Deák, P. A., Kiss, G., Gerlei, Z., Kobori, L., and 5 others 2017. “Management of Portal Hypertension after Liver Transplantation,” Transplantation Proceedings 49(7): 1530-1534).

Intermittent subcomplete relief of portal hypertension by an adjustable extrahepatic rather than nonadjustable intrahepatic and unremovable shunt can significantly reduce the risk of acute liver failure due to hepatic ischemia, hypertrophy of and over-dependency upon the hepatic artery. Direct piping of antimicrobials and anticoagulants into the shunt prevents blockage. To lessen control system complexity, these can be established as automatic slow drips with guidance for replenishment of the reservoirs.

2b(2)(h)(h3). The ‘Tuned’ Shunt in the Relief of Pulmonary Arterial Hypertension

In advanced if not end-stage heart disease, where the need for transplantation is clearly indicated, methods for the remediation of pulmonary hypertension are addressed in sections 2b(1)(b)(b5), entitled Double Heart Metered Compound vascular bypass, or Switched-organ Transplantation [and so on], as well as in the sections above addressing heart-lung transplantation, pulmonary resistance, and tuned shunts. Here the remediation of pulmonary hypertension is dealt with through tuned shunt mediated pressure relief outside the context of a need for organ replacement. Gene therapy in the context of an implanted automatic disorder response system is addressed in the preceding section pertaining to portal hypertension, and in more general medical terms in section 2a, entitled Background of the Invention. General, as well as in other sections to follow.

Unless the condition of the patient precludes treatment which addresses the primary cause of the pathology, may even conceal the cause, such secondary measures alone are meant only to ameliorate the injury caused by the increased pressure, absent treatment that goes if discernible goes to the etiological basis for the hypertension, the relief of excessive pressure in itself is not a cure. Pulmonary hypertension is also addressed above in section 2a, entitled Background of the Invention. General.

Pulmonary hypertension can represent back pressure in the main pulmonary artery due to pulmonary vascular resistance treatable by the direct pipeline targeting of a locally or topically effective antihypertensive drug such as nitroglycerin through the accessory channels of a diversion jacket at the pulmonary trunk, or a one-way passive pressure relief valve, or a negative feedback-driven servochoke. In the very old, very young, and others not dependable for adherence to a prescription regimen, systemic hypertension is treatable with conventional drugs by direct delivery into the systemic circulation. The drug with the quickest onset of action for the treatment of pulmonary hypertension is the vasodilator nitroglycerin when administered intravenously (Physicians' Desk Reference 2022, Op cit., above in section 2a, entitled Background. General).

Pulmonary hypertension can also result from vascular disease or increased resistance to the blood supply to the lungs due to intrapulmonary vasoconstriction, a condition such as pulmonary fibrosis, a congenital heart defect (see, for example, Zhou, Z., Gu, Y., Zheng, H., Yan, C., Liu, Q., and 6 others 2023. “Interventional Occlusion of Large Patent Ductus Arteriosus in Adults with Severe Pulmonary Hypertension,” Online, MDPI [Multidisciplinary Digital Publishing Institute] Journal of Clinical Medicine (Basel, Switzerland) 12(1): 354) or inflammation of the intrapulmonary arteries. A tuned shunt placed to decompress a hypertensive pulmonary artery by passing a volume of blood from that vessel into the superior vena cava such as shown in FIG. 22F can incorporate a pressure sensor.

Options in severe pulmonary hypertension are lung or lung and heart transplantation to include either or both lungs, these operations performed using the metered compound vascular method addressed above in sections 2b(1)(b)(b9), entitled Single Living and Single or Double Deceased Donor Metered Compound Vascular Bypass, or Switched-organ, Lung Transplantation, and 2b(1)(b)(b10), entitled Metered Compound Vascular Bypass, or Switched-organ, Heart-Lung Transplantation (Hu, X., Ding, N., Songchen, W., Wang, R., Chen, J., and 5 others 2023. “Lung Transplantation for Pulmonary Arterial Hypertension: Optimized Referral and Listing Based on an Evolving Disease Concept,” Online, MDPI [Multidisciplinary Digital Publishing Institute] Journal of Cardiovascular Development and Disease (Basel, Switzerland) 10(8): 350; Kolaitis, N. A. 2023. “Lung Transplantation for Pulmonary Arterial Hypertension,” Chest (New York, New York) S00123692(23): 00649-00699; Deshwal, H., Weinstein, T., Sulica, R. 2021. “Advances in the Management of Pulmonary Arterial Hypertension,” Online, Journal of Investigative Medicine (Thousand Oaks, California) 69(7): 1270-1280; Hascoët, S., Pontailler, M., Le Pavec, J., Savale, L., Mercier, O., and 11 others 2021. “Transplantation for Pulmonary Arterial Hypertension with Congenital Heart Disease: Impact on Outcomes of the Current Therapeutic Approach Including a High-priority Allocation Program,” Online, American Journal of Transplantation (New York, New York) 21(10): 3388-3400; Hwalek, A., Rosenheck, J. P., and Whitson, B. A. 2021. “Lung Transplantation for Pulmonary Hypertension,” Online, Journal of Thoracic Disease (Hong Kong, China) 13(11): 6708-6716; Le Pavec, J., 1, Feuillet, S., Mercier, O., Pauline, P., Dauriat, G., and 14 others 2021. “Lung and Heart-lung Transplantation for Children with PAH [pulmonary arterial hypertension]: Dramatic Benefits from the Implementation of a High-priority Allocation Program in France,” Journal of Heart and Lung Transplantation (New York, New York) 40(7): 652-661; Sommer, W. and Warnecke, G. 2021. “Lung Transplantation for Pediatric Pulmonary Arterial Hypertension-Quo Vadis?,” Online, Cardiovascular Diagnosis and Therapy (Hangzhou, China) 11(4): 1178-1189; Antończyk, R., Stącel, T., Urlik, M., Latos, M., Kret, M., and 8 others 2020. “Single Lung Transplant vs Double Lung Transplant: A Single-center Experience with Particular Consideration for Idiopathic Pulmonary Arterial Hypertension,” Online, Transplantation Proceedings (New York, New York) 52(7): 2138-2142; Eadington, T., Santhanakrishnan, K., and Venkateswaran, R. 2020. “Heart-Lung Transplantation for Idiopathic Pulmonary Arterial Hypertension and Giant Artery Aneurysm-Case Report,” Online, Journal of Cardiothoracic Surgery (London, England) 15(1): 169; Villavicencio, M. A., Axtell, A. L., Osho, A., Astor, T., Roy, N., and 6 others 2018. “Single-versus Double-lung Transplantation in Pulmonary Fibrosis: Impact of Age and Pulmonary Hypertension,” Online, Annals of Thoracic Surgery (Amsterdam, North Holland) 106(3): 856-863; Lopez-Meseguer, M., Quezada, C. A., Ramon, M. A., Lázaro, M., Dos, L., and 5 others 2017. “Lung and Heart-Lung Transplantation in Pulmonary Arterial Hypertension,” Online, PLoS [Public Library of Science] One 12(11): e0187811, correction 13(1): e0192100), and interventional cardiology (Hirakawa, K., Yamamoto, E., Takashio, S., Hanatani, S., Araki, S., and 5 others 2022. “Balloon Pulmonary Angioplasty in Chronic Thromboembolic Pulmonary Hyperextension,” Cardiovascular Intervention and Therapeutics 37(1): 60-65; Gurevich, S. and Prins, K. W. 2021. “The Evolving Role of Interventional Cardiology in the Treatment of Pulmonary Hypertension,” Online, Catheterization and Cardiovascular Interventions (New York, New York) 97(4): e446-e453).

A remote sensor along the bloodstream can indicate the type of pulmonary hypertension ((Tomaszewski, M., Malkowska, P., Sierawska, O., Hrynkiewicz, R., Mroczek, E., and 5 others 2022. “CTLA-4 [T cell carried immune suppressor protein] Expression is a Promising Biomarker of Idiopathic Pulmonary Arterial Hypertension and Allows Differentiation of the Type of Pulmonary Hypertension,” Online, MDPI [Multidisciplinary Digital Publishing Institute] International Journal of Molecular Sciences (Basel, Switzerland) 23(24): 15910).

The ‘opposite’ cause is excessive force of ejection from the right ventricle, and numerous other conditions can be responsible or involved. Usually, the etiology is not simple or singular, and an ultimate cause indiscernible or inconclusive. Impairment of the right ventricle and/or mitral valve may be primary but almost always is secondary to the condition, which presents as a progressive stenosis of the intrapulmonary arteries due to impaired mitosis and contractile capability of the smooth muscle cells in the walls of the arteries. More detailed diagnostics follow.

The potential cause or causes for pulmonary hypertension multifactorial where no one factor is primary, and the ultimate cause or causes in the specific patient might never be determined to satisfaction, albeit only partial but immediate amelioration, pressure relief should be accomplished without delay, the effort to accomplish a proper diagnosis and therapy continued. Means for adjusting local blood pressure described herein include servochokes, servosphincters, servodampers, and diversion servovalves. These can be controlled on an intermittent or continuous basis by the implanted master controller in accordance with the prescription-program. Such means are no less applicable to the fine tuning of any type transplant once accomplished.

While pressure reduction is accomplished through pulmonary vasodilators or reduction or elimination of innate shunting associated with congenital heart malformity (see, for example, Arshad, H. B. and Duarte, V. E. 2021. “Evaluation and Management of Pulmonary Arterial Hypertension in Congenital Heart Disease,” Online, Methodist DeBakey Cardiovascular Journal 17(2): 145-151; Brida, M., Nashal, H., and Gatzoulis, M. A. 2020. “Pulmonary Arterial Hypertension: Closing the Gap in Congenital Heart Disease,” Current Opinion in Pulmonary Medicine 26(5): 422-428; Chida-Nagai, A., Sagawa, K., Tsujioka, T., Fujimoto, T., Taniguchi, K., and 6 others 2020. “Pulmonary Vasodilators Can Lead to Various Complications in Pulmonary “Arterial” Hypertension Associated with Congenital Heart Disease,” Online, Heart and Vessels 35(9): 1307-1315; Pascall, E. and Tulloh, R. M. 2018. “Pulmonary Hypertension in Congenital Heart Disease,” Online, Future Cardiology 14(4): 343-353; Couperus, L. E., Henkens, I. R., Jongbloed, M. R. M., Hazekamp, M. G., Schalij, M. J., and Vliegen, H. W. 2016. “Tailored Circulatory Intervention in Adults with Pulmonary Hypertension Due to Congenital Heart Disease,” Netherlands Heart Journal 24(6): 400-409) or septostomy (see, for example, Allcock, R. J., O'Sullivan, J. J., and Corris, P. A. 2003. “Atrial Septostomy for Pulmonary Arterial Hypertension,” Heart 89(11): 1344-1347) rather than by diversion of some blood from the main pulmonary artery to another vessel, the reduction in pulmonary arterial pressure by reducing blood volume is not new.

That the implanted automatic prosthetic disorder response system can directly pipe-target medication to any point in the body, and if necessary, extract any residue before it can pass on through the circulation considerably reduces the risk of encouraging the development of antimicrobial resistant pathogens (see, for example, Li, J. W., Guo, L. W., Tao, S. Y., Deng, Y. H., Yang, C., and Qiao, F. 2022. “Analysis of Healthcare-associated Infection in Patients with Pulmonary Arterial Hypertension Associated with Congenital Heart Disease in PICU [pediatric intensive care unit]: Evidence from a Tertiary Hospital in Western China,” Online, Frontiers in Pediatrics 10:1076618)/Secondary effects of pulmonary hypertension include edema of the lower legs, syncope, dyspnea, tachycardia, malaise, anorexia, abdominal and thoracic pain, and given a marked reduction in the delivery of blood to be oxygenated, a chronic hypoxemia that imparts a bluish tinge to the skin, or cyanosis. The mechanical relief of pulmonary hypertension is intended to ameliorate these sequelae in coordination with the automatic release of medication directly pipeline targeted to the treatment site through the valve-jacket accessory channels.

Long-term survival is poor as to require a lung transplant, or if the right ventricle and/or mitral valve are impaired, a heart-lung transplant. Relieving the pressure in the pulmonary arteries is not a cure but rather a palliative measure which combined with oxygen therapy and drugs, forestalls or ameliorates the foregoing secondary sequelae of the disease, which left untreated, is fatal. If inadequate to eliminate the eventual need for a transplant indefinitely, the approach is nonetheless an effective bridging strategy until replacement lungs, or lungs and a heart, are found. The partial diversion of blood from the main pulmonary artery to the proximal descending aorta is described below in Section 6.

Provided the volume of blood to be diverted is expected to remain substantially constant or if not, then compensable by adjusting the coordinated dose of medication, conventional valves should prove satisfactory. If the condition is significantly variable, the placement of vascular servovalves will eliminate the need for reentry. Drug delivery to nontubular target tissue is through a nonjacketing side-entry connector, described in U.S. Pat. No. 11,013,858, entitled Nonjacketing Side-entry Connectors and Prosthetic Disorder Response Systems, publication number 20160051806.

Implanted prosthetic disorder response systems are described in U.S. Pat. No. 11,759,186, entitled Ductus Side-entry Jackets and Prosthetic Disorder Response Systems, filed on 8 Jun. 2018 and its parent application Ser. No. 14/121,365, filed on 25 Aug. 2014 or the continuation-in-6part thereto, Ser. No. 15/998,002. Vascular valves are fixed in aperture cross-section and divert a fixed volume of blood or urine, depending upon the type of valve, by diverting or choking flow. In contrast, vascular servovalves are continuously variable in aperture cross-section.

Placed under the control of an implanted microcontroller- or in a more comprehensive system to deal with comorbidities, a microprocessor taking its inputs from physiological sensors positioned to provide data indicative of the instant condition of the affected function of functions, vascular valves divert a fraction of the blood to another vessel. Reduction in blood pressure through diversion is a more complex example of the use of vascular valves to correct a complete transposition of the great arteries without entry into the heart, where diversion is constant and invariable.

Dynamically sensor data controller commanded responsive servovalves are preferably supported by a fully or closed-skin implanted control system to immediately target medication from implanted reservoirs to the treatment site or sites automatically in coordination with alteration in the pertinent physiological indicia according to the prescription-program. While to treat less complex disease, fixed vascular valves are usable without such support, servovalves require an automatic control system to adjust instantly in rate of flow-through as well as have the coordinated support of medication. In figs. X and X showing a servovalve, the diversion of blood or urine is through the mainline, and the channeling of medication to and through the valve is along one or more accessory channels to each valve.

NAME THE DISEASE PHENOMENA physiological parameters TO BE continuously MONITORED BY SENSORS TO ADJUST THE VOLUME OF BLOOD TO BE DIVERTED AND to detect the SYMPTOMS TO BE TREATED THROUGH THE COORDINATED USE OF SPECIFIED DRUGS.

newpatinsert.docx follows.

It is instructive that the spontaneous adaptation to pulmonary hypertension is the development of collateral circulation to relieve the pressure; (see, for example; Kepez, A., Mutlu, B., Paudel, A., Ileri, C., Atas, H., and Yildizeli, B. 2018. “Prevalence of Coronary Artery to Pulmonary Artery Collaterals in Patients with Chronic Thromboembolic Pulmonary Hypertension: Retrospective Analysis from a Single Center,” Thoracic and Cardiovascular Surgeon 66(2): 180-186; Lee, N. S., Auger, W. R., Pretorius, V., Blanchard, D. G., and Daniels, L. B. 2014. “Coronary Artery-to- pulmonary Artery Collaterals in Chronic Thromboembolic Pulmonary Hypertension,” Circulation. Cardiovascular Imaging 7(6): 962-966).

However, the collaterals can encroach upon coronary arteries, creating an equally ominous condition (see, for example, Sugiyama, K., Koizumi, N., and Ogino, H. 2018. “Severe Compression of the Left Main Coronary Artery in a Patient with Chronic Thromboembolic Pulmonary Hypertension,” Annals of Thoracic and Cardiovascular Surgery 19;24(5): 251-254). The use of a vascular servovalve to pass off some blood to another vessel is effectively a synthetic collateral, but one that will not encroach upon a coronary artery. The treatment of pulmonary hypertension follows from the subtype, defined and organized by the World Health Organization into five groupings.

The first group consists of pulmonary arterial hypertension whether hereditary, drug-induced, associated with a connective tissue disorder, congenital heart disease, hepatic hypertension, hematological infection or disorder, or malformation such as intimal hyperplasia of the veins and capillaries of the lungs (see, for example, Mertens, T. J., Hanmandlu, A., Tu, L., Phan, C., Collum, S. D., and 13 others 2018. “Switching-off Adora2b in Vascular Smooth Muscle Cells Halts the Development of Pulmonary Hypertension,” Online, Frontiers in Physiology 9:555; Prins, K. W. and Thenappan, T. 2016. “World Health Organization Group I Pulmonary Hypertension: Epidemiology and Pathophysiology,” Cardiology Clinics 34(3): 363-374; Karmouty-Quintana, H., Philip, K., Acero, L. F., Chen, N. Y., Weng. T., and 11 others 2015. “Deletion of ADORA2B from Myeloid Cells Dampens Lung Fibrosis and Pulmonary Hypertension,” Federation of American Societies for Experimental Biology Journal 29(1): 50-60; Ventetuolo, C. E. and Klinger, J. R. 2012. “WHO Group|Pulmonary Arterial Hypertension: Current and Investigative Therapies,” Progress in Cardiovascular Diseases 55(2): 89-103).

The second group present pulmonary hypertension as secondary to left ventricular systolic or diastolic dysfunction or left valvular defects, or congenital heart malformity (see, for example, Koulova, A., Gass, A. L., Patibandla, S., Gupta, C. A., Aronow, W. S., and Lanier, G. M. 2017. Management of Pulmonary Hypertension from Left Heart Disease in Candidates for Orthotopic Heart Transplantation,” Journal of Thoracic Disease 9(8): 2640-2649; Clark, C. B. and Horn, E. M. 2016. “Group 2 Pulmonary Hypertension: Pulmonary Venous Hypertension: Epidemiology and Pathophysiology,” Cardiology Clinics 34(3): 401-411; Rosenkranz, S., Gibbs, J. S., Wachter, R., De Marco, T., Vonk-Noordegraaf, A., and Vachiery, J. L. 2016. “Left Ventricular Heart Failure and Pulmonary Hypertension,” European Heart Journal 37(12): 942-954).

The third group exhibits the condition due to intrinsic lung disease, disturbed sleep, or chronic hypoxia (see, for example, Cano-Jiménez, E., Hernández González, F., and Peloche, G. B. 2018. “Comorbidities and Complications in Idiopathic Pulmonary Fibrosis,” Medical Sciences (Basel, Switzerland) 6(3). pii: E71; Collum, S. D., Amione-Guerra, J., Cruz-Solbes, A. S., DiFrancesco, A., Hemandez, A. M., and 4 others 2017. “Pulmonary Hypertension Associated with Idiopathic Pulmonary Fibrosis: Current and Future Perspectives,” Canadian Respiratory Journal 2017:1430350; Klinger, J. R. 2016. “Group III Pulmonary Hypertension: Pulmonary Hypertension Associated with Lung Disease: Epidemiology, Pathophysiology, and Treatments,” Cardiology Clinics 34(3): 413-433; Adir, Y. and Harari, S. 2014. “Pulmonary Hypertension Associated with Chronic Obstructive Lung Disease and Idiopathic Pulmonary Fibrosis,” Current Opinion in Pulmonary Medicine 20(5): 414-420; Seeger, W., Adir, Y., Barbera, J. A., Champion, H., Coghlan, J. G., and 10 others 2013. “Pulmonary Hypertension in Chronic Lung Diseases,” Journal of the American College of Cardiology 62(25 Supplement): D109-116).

The fourth as the result of chronic pulmonary thromboembolism (see, for example, Kim, N. H. 2016. “Group 4 Pulmonary Hypertension: Chronic Thromboembolic Pulmonary Hypertension: Epidemiology, Pathophysiology, and Treatml Sent,” Cardiology Clinics 34(3): 435-441; Tousoulis, D. 2018. “Chronic Thromboembolic Pulmonary Hypertension. What We Are Missing?,” Hellenic Journal of Cardiology (Amsterdam, North Holland, Netherlands) 59(1): 1-2; Kepez, A., Mutlu, B., Paudel, A., Ileri, C., Atas, H., and Yildizeli, B. 2018, Op cit.; Robbins, I. M., Pugh, M. E., and Hemnes, A. R. 2017. “Update on Chronic Thromboembolic Pulmonary Hypertension,” Trends in Cardiovascular Medicine 27(1): 29-37; Stricker, H. 2016. “Chronic Thromboembolic Pulmonary Hypertension—A Diagnostic and Therapeutic Update,” VASA. Zeitschrift für Gefasskrankheiten 45(3): 195-199; Hoeper, M. M. 2015. “Pharmacological Therapy for Patients with Chronic Thromboembolic Pulmonary Hypertension,” European Respiratory Review 24(136): 272-282; Mackenzie Ross, R. V., Toshner, M. R., Soon, E., Naeije, R., and Pepke-Zaba, J. 2013. “Decreased Time Constant of the Pulmonary Circulation in Chronic Thromboembolic Pulmonary Hypertension,” American Journal of Physiology. Heart and Circulatory Physiology 305(2): H259-H26).

Conventional treatments such as pulmonary endarterectomy (see, for example, D'Armini, A. M., Morsolini, M., Mattiucci, G., Grazioli, V., Pin, M., and 4 others 2016. “Chronic Thromboembolic Pulmonary Hypertension: From Transplantation to Distal Pulmonary Endarterectomy,” Journal of Heart and Lung Transplantation (New York, New York) 35(6): 827-831; Madani, M. M. 2016. “Surgical Treatment of Chronic Thromboembolic Pulmonary Hypertension: Pulmonary Thromboendarterectomy,” Methodist DeBakey Cardiovascular Journal 12(4): 213-218) and angioplasty (see, for example, Menon, K., Sutphin, P. D., Bartolome, S., Kalva, S. P., and Ogo, T. 2018. “Chronic Thromboembolic Pulmonary Hypertension: Emerging Endovascular Therapy,” Cardiovascular Diagnosis and Therapy 8(3): 272-278; Karyofyllis, P., Papadopoulou, V., Voudris, V., Matsubara, H. 2018. “Balloon Pulmonary Angioplasty in Patients with Thromboembolic Pulmonary Hypertension,” Current Treatment Options in Cardiovascular Medicine 20(2): 13; Rivers-Bowerman, M. D., Zener, R., Jaberi, A., de Perrot, M., Granton, J., Moriarty, J. M., and Tan, K. T. 2017. “Balloon Pulmonary Angioplasty in Chronic Thromboembolic Pulmonary Hypertension: New Horizons in the Interventional Management of Pulmonary Embolism,” Techniques in Vascular and Interventional Radiology 20(3): 206-215) pose risks less likely with synthetic collaterals.

The fifth (see, for example, Kalantari, S. and Gomberg-Maitland, M. 2016. “Group 5 Pulmonary Hypertension: The Orphan's Orphan Disease,” Cardiology Clinics 34(3): 443-449) consists of blood disorders not included in the first group, such as an:
a. Anemia (see, for example, Ramakrishnan, L., Pedersen, S. L., Toe, Q. K., Quinlan, G. J., and Wort, S. J. 2018. “Pulmonary Arterial Hypertension: Iron Matters,” Online, Frontiers in Physiology 9:641; Kurdyukov, S., Eccles, C. A., Desai, A. A., Gonzalez-Garay, M., Yuan, J. X., and 3 others 2018. “New Cases of Glucose-6-phosphate dehydrogenase Deficiency in Pulmonary Arterial Hypertension,” Online, PLoS [Public Library of Science] One 13(8): e0203493; Mathew, R., Huang, J., Wu, J. M., Fallon, J. T., and Gewitz, M. H. 2016. “Hematological Disorders and Pulmonary Hypertension,” World Journal of Cardiology 8(12): 703-718; Machado, R. F. and Farber, H. W. 2013. “Pulmonary Hypertension Associated with Chronic Hemolytic Anemia and Other Blood Disorders,” Clinics in Chest Medicine 34(4): 739-752),
b. Systemic disorders which impair the organs, such as sarcoidosis (see, for example, Duong, H. and Bonham, C. A. 2018. “Sarcoidosis-associated Pulmonary Hypertension: Pathophysiology, Diagnosis, and Treatment,” Clinical Pulmonary Medicine 25(2): 52-60; Ramachandraiah. V., Aronow, W., and Chandy, D. 2017. “Pulmonary Sarcoidosis: An Update,” Postgraduate Medicine 129(1): 149-158; Huitema, M. P., Grutters, J. C., Rensing, B. J. W. M., Reesink, H. J., and Post. M. C. 2016. “Pulmonary Hypertension Complicating Pulmonary Sarcoidosis,” Netherlands Heart Journal 24(6): 390-399; Israel-Biet, D. and Valeyre, D. 2013. “Diagnosis of Pulmonary Sarcoidosis,” Current Opinion in Pulmonary Medicine 19(5): 510-515; Cordova, F. C. and D'Alonzo, G. 2013. “Sarcoidosis-associated Pulmonary Hypertension,” Current Opinion in Pulmonary Medicine 19(5): 531-537),
c. Metabolic disorders which cause or affect pulmonary hypertension include:
1. Glycogen storage (see, for example, Li, H. P., Xie, W. M., Huang, X., Lu, X., Zhai, Z. G., Zhan, Q. Y., and Wang, C. 2018. “Pulmonary Hypertension in Glycogen Storage Disease Type II,” Chinese Medical Journal (English edition) 131(11): 1375-1376; Lee, T. M., Berman-Rosenzweig, E. S., Slonim, A. E., and Chung, W. K. 2011. “Two Cases of Pulmonary Hypertension Associated with Type III Glycogen Storage Disease,” Journal of Inherited Metabolic Disorders Reports 1:79-82; Humbert, M., Labrune, P., and Simonneau, G. 2002. “Severe Pulmonary Arterial Hypertension in Type 1 Glycogen Storage Disease,” European Journal of Pediatrics 161 Supplement 1: S93-596).
2. Thyroid function (see, for example, Richter, M. J., Sommer, N., Schermuly, R., Grimminger, B., Seeger, W., and 3 others 2016. “The Prognostic Impact of Thyroid Function in Pulmonary Hypertension,” Journal of Heart and Lung Transplantation (New York, New York) 35(12): 1427-1434; Scicchitano, P., Dentamaro, I., Tunzi, F., Ricci, G., Carbonara, S., and 4 others 2016. “Pulmonary Hypertension in Thyroid Diseases,” Endocrine 54(3): 578-587; Vakilian, F., Attaran, D., Shegofte, M., Lari, S., and Ghare, S. 2016. “Assessment of Thyroid Function in Idiopathic Pulmonary Hypertension,” Research in Cardiovascular Medicine 5(2): e29361; Marvisi, M., Balzarini, L., Mancini, C., and Mouzakiti, P. 2013. “Thyroid Gland and Pulmonary Hypertension. What's the Link?,” Panminerva Medica 55(1): 93-97; Silva, D. R.1, Gazzana, M. B., John, A. B., Siqueira, D. R., Maia, A. L., and Barreto, S. S. 2009. “Pulmonary Arterial Hypertension and Thyroid Disease,” Jornal Brasileiro de Pneumologia (Brasilia Mexico Distrito Federal, Brazil) 35(2): 179-185).
d. Chronic kidney failure (see, for example, Peter, I. D., Asani, M. O., Aliyu, I., and Obiagwu, P. N. 2018. “Elevated Mean Pulmonary Artery Pressure and Right Ventricular Dysfunction in Children with Chronic Kidney Disease,” Journal of Cardiovascular Echography 28(2): 109-113; Zhang, Q., Wang, L., Zeng, H., Lv, Y., and Huang, Y. 2018. “Epidemiology and Risk Factors in CKD [chronic kidney disease] Patients with Pulmonary Hypertension: A Retrospective Study,” Online, BioMed Central Nephrology 19(1): 70; Suresh, H., Arun, B. S., Moger, V., Vijayalaxmi, P. B., and Murali Mohan, K. T. K. 2018. “A Prospective Study of Pulmonary Hypertension in Patients with Chronic Kidney Disease: A New and Pernicious Complication,” Indian Journal of Nephrology 28(2): 127-134; O'Leary, J. M., Assad, T. R., Xu, M., Birdwell, K. A., Farber-Eger, E., and 3 others 2017. “Pulmonary Hypertension in Patients with Chronic Kidney Disease: Invasive Hemodynamic Etiology and Outcomes,” Pulmonary Circulation 7(3): 674-683; Thenappan, T. 2017. “Pulmonary Hypertension in Chronic Kidney Disease: A Hemodynamic Characterization,” Pulmonary Circulation 7(3): 567-568; Li, Z., Liang, X., Liu, S., Ye, Z., Chen, Y., and 5 others 2014. “Pulmonary Hypertension: Epidemiology in Different CKD [chronic kidney disease] Stages and Its Association with Cardiovascular Morbidity,” Online, PLoS [Public Library of Science] One 9(12): e114392; Sise, M. E., Courtwright, A. M., and Channick, R. N. 2013. “Pulmonary Hypertension in Patients with Chronic and End-stage Kidney Disease,” Kidney International 84(4): 682-692),
e. Physical obstruction of the pulmonary arteries by tumors (see, for example, Morin-Thibault, L. V., Wiseman, D., Fortin, M., Couture, C., and Provencher, S. 2018. “Pulmonary Micro-tumor Emboli Resulting in Paradoxical Emboli: A Case Report,” Pulmonary Circulation 8(2): 2045893218754853; Okazaki, S., Abe, T., Takayanagi, N., Yasuda, M., Sakai, F., Kobayashi, K., and Kato, S. 2018. “Pulmonary Tumor Embolism Due to Squamous Cell Carcinoma of the Uterine Cervix: A Case Report,” In Vivo (Athens, Greece) 32(2): 337-343; Tsujimoto, N., Miyoshi, A., Mimura, M., Naka, T., Yamasaki, M., and Yokoi, T. 2018. “Case Report: Pulmonary Tumor Thrombotic Microangiopathy in a Cervical Cancer Patient,” Gynecologie and Obstetric Investigation 83(1): 99-104; Latchana, N., Daniel, V. C., Gould, R. W., and Pollock, R. E. 2017. “Pulmonary Tumor Embolism Secondary to Soft Tissue and Bone Sarcomas: A Case Report and Literature Review,” World Journal of Surgical Oncology 15(1): 168; Mury, C., Schneider, A. G., Nobile, A., Rotman, S., and Liaudet, L. 2015. “Acute Pulmonary Hypertension Caused by Tumor Embolism: A Report of Two Cases,” Pulmonary Circulation 5(3): 577-579; Brock, S., Fuller, C., and Iveson, T. 2000. “Pulmonary Tumour Emboli: A Difficult Ante-mortem Diagnosis,” Clinical Oncology (Royal College of Radiologists) 12(1): 56-57).

The number of causes, overlap among these, distinguishing consequences from the causes for pulmonary hypertension, and complex interrelations with sequelae make diagnosis difficult, leaving the pulmonary arterial hypertension itself as the central target of therapy (see, for example, Oldroyd, S. H., Manek, G., Saankari, A., and Bhardwaj, A. 2023. “Pulmonary Hypertension,” Online, Treasure Island, Florida: StatPearls Publishing Co.; Rubin, L. J. and Hopkins, W. 2018. “Classification and Prognosis of Pulmonary Hypertension in Adults,” Wolters Kluwer UpToDate Online at https://www.uptodate.com/contents/classification-and-prognosis-of-pulmonary-hypertension-in-adults; Pulmonary Hypertension News 2018. “Pulmonary Hypertension WHO Classification” Bionews Feeds Online at https://pulmonary hypertensionnews.com/pulmonary-hypertension-who-classification/; Mak, S. M., Strickland, N., and Gopalan, D. 2017. “Complications of Pulmonary Hypertension: A Pictorial Review,” British Journal of Radiology 90(1070): 20160745; Jutant, E. M. and Humbert, M. 2016. “Pulmonary Hypertension: Definition, Classification and Treatments,” (in French with abstract at Pubmed) Biologie Aujourd'hui [Journal of the French Society of Biology] 210(2): 53-64; Rose-Jones, L. J. and Mclaughlin, V. V. 2015. “Pulmonary Hypertension: Types and Treatments” Current Cardiology Reviews 11(1): 73-79; Montani D, Gunther S, Dorfmuller P, Perros F, Girerd B, and 8 others 2013. “Pulmonary Arterial Hypertension,” Orphanet Journal of Rare Diseases 8:97).

The cause of causes of pulmonary arterial hypertension may be intrinsic in the lungs, as the result of pulmonary maldevelopment; thus primary, or secondary, in that a process or processes external to the lungs upon which the lungs depend, such as a connective tissue or metabolic disorder, is or are absent, insufficient, or defective. The causes numerous and interrelated, in some cases, the cause will be equivocal, multiple, or undecipherable (see, for example, Bazmpani, M. A., Arsos, G., Zarogoulidis, P., Doumas, A., Dimitroulas, T., and 6 others 2018. “A Case of Sarcoidosis-associated Pulmonary Hypertension Masquerading as Chronic Thromboembolic Pulmonary Hypertension,” Pulmonary Circulation 8(3): 2045894018768289; Huitema, M. P., Grutters, J. C., Rensing, B. J. W. M., Reesink, H. J., and Post, M. C. 2016, Op cit.; Post, M. C. and van der Wall, E. E. 2016. “Pulmonary Hypertension: The Importance of a Multidisciplinary Approach,” Netherlands Heart Journal 2016 24(6): 369-371; Bazan, I. S. and Fares, W. H. 2015. “Pulmonary Hypertension: Diagnostic and Therapeutic Challenges,” Therapeutics and Clinical Risk Management 11:1221-1233). If the cause is not intrinsic in the lungs, the primary cause may or may not be treatable.

As indicated above, given the difficulty or the lack of a satisfactory diagnosis and complexity of a solution devised to achieve comprehensive therapy that goes to the etiological cause or causes, treatment is best concentrated on the pulmonary hypertension, established causes treated to the extent that the fully implanted system will accommodate, with the balance of treatment relegated to the clinic.

For simplicity and economy, a practical prosthetic disorder response system is favorably limited to the treatment of the pulmonary hypertension itself and any cause or causes that precipitate episodes fulminant and injurious as to demand immediate suppression. Both before and after transplantation, the incorporation of complexity based upon an uncertain diagnosis will not only result in a system which is obtrusive but one likely compromised in the effectiveness of a more concerted response. Concentrating on the immediate and essential, the system is devised to provide a forceful and immediate response to reversals of sudden onset, more comprehensive treatment relegated to the clinic where diagnostic and therapeutic tools are unlimited. Ultimate etiology often undecipherable, obscure, uncertain, or complex, treatment prior to the advent of an implantable response system was usually limited to treating the symptoms anyway.

When it is known that an implant system will respond to symptoms instantly and forcefully-before these are allowed to progress to the point where the patient experiences the symptoms and so that consequent injury will have been minimized, the prospects for success, hence, the inducement to proceed with transplantation is supported. When the system can be devised to present an effective response to sickle cell anemia, for example, there will be greater incentive to replace lungs that had been damaged by that disease; awareness that the transplant will have immediate intracorporeal support serves as a positive inducement to proceed with a transplant.

In general, pulmonary hypertension can be ameliorated by electrical neuromodulation applied to or the release of drugs into the right heart and by drawing or tapping off just enough blood from the main pulmonary artery to reduce the pressure (see, for example, Wang, J., Feng. W., Li, F., Shi, W., Zhai, C., and 7 others 2019. “SphK1/SIP [sphingosine kinase 1/sphingosine-1-phosphate] Mediates TGF-β1 [transforming growth factor beta 1 cytokine]-induced Proliferation of Pulmonary Artery Smooth Muscle Cells and Its Potential Mechanisms,” Pulmonary Circulation 9(1): 2045894018816977; Oldroyd, S. H. and Bhardwaj. A. 2018, Op cit.; Goldberg, A. B., Mazur, W., and Kalra, D. K. 2017. “Pulmonary Hypertension: Diagnosis, Imaging Techniques, and Novel Therapies,” Cardiovascular Diagnosis and Therapy 7(4): 405-417).

Due to the forcefulness of cardiac action, to accomplish this by taking blood from the right heart is discouraged. By contrast, a side-entry servovalve-jacket completely encircles the main pulmonary artery, is lined to accommodate its pulsation, which is less intense than the contraction of the heart, without injuring the adventitia, and under the tight control of an implant microcontroller, vents the blood through the small mainline, which fully enclosed, cannot result in problem bleeding, especially problematic when anticoagulants are used.

That this approach is fully implanted, self-sufficient, and ambulatory means that the system dynamically responds to the pulmonary pressure by dispensing drugs, electrostimulatory neuromodulation, and/or venting blood immediately to restore the pressure to as close to normal as possible. This immediacy is significant, because the pressure can vary with a change in body position, much less during exercise. While the disease is progressive, the consequence of immediate restoration to the set point should forestall the need for a lung transplant.

In drug regimen-competent patients, symptoms readily treated with oral medication, such as analgesics to reduce the pain of vaso-occlusive crises in sickle cell disease (a special type of anemia-see, for example, Alsaied, T., Niss. O., Powell, A. W., Fleck, R. J., Cnota, J. F., and 4 others 2018. “Diastolic Dysfunction is Associated with Exercise Impairment in Patients with Sickle Cell Anemia,” Pediatric Blood and Cancer 65(8): e27113; Ahmed, M. and Miller, E. 2018. “Macrophage Migration Inhibitory Factor (MIF) in the Development and Progression of Pulmonary Arterial Hypertension,” Global Cardiology Science and Practice 2018(2): 14; Shilo, N. R. and Morris, C. R. 2017. “Pathways to Pulmonary Hypertension in Sickle Cell Disease: The Search for Prevention and Early Intervention,” Expert Review of Hematology 10(10): 875-890; Galie. N., Humbert. M., Vachiery, J. L., Gibbs, S., Lang. I., and 16 others 2016. “ESC/ERS [European Society of Cardiology/European Respiratory Society] 2015 Guidelines for the Diagnosis and Treatment of Pulmonary Hypertension,” Online, European Heart Journal 37(1): 67-119; Gordeuk, V. R., Castro. O. L., and Machado, R. F. 2016. “Pathophysiology and Treatment of Pulmonary Hypertension in Sickle Cell Disease,” Blood 127(7): 820-828; Hayes, M. M., Vedamurthy, A., George, G., Dweik, R., Klings. E. S., and 4 others 2014. “Pulmonary Hypertension in Sickle Cell Disease,” Annals of the American Thoracic Society 11(9): 1488-1489; Ataga, K. I. and Klings, E. S. 2014. “Pulmonary Hypertension in Sickle Cell Disease: Diagnosis and Management,” Hematology. American Society of Hematology. Education Program. 2014(1): 425-431), are favorably eliminated from the implant system, reducing unnecessary complexity and expense.

Before transplantation, the main consideration is the suppression of the pulmonary hypertension and contributary factors dealt with simply. After transplantation, the circumstances that would cause the transplant to fail may become important enough to affect the decision to perform the transplant from the outset. In either case, more challenging etiology is relegated to the clinic. That is, the decision to proceed with a transplant takes into consideration the extent to which the implant system can be configured and programmed to suppress causative factors treated more simply with drugs or by activating an implanted neuromodulator. Etiological factors differing greatly in the challenge presented to an implant system, whether additional capability to deal with etiology is incorporated into the implant system depends upon the burden imposed by having to visit the clinic and the cost to provide the additional capability, as well as the availability of the technology. Where the automatic sensing and release of a drug is required, the causative factor can be dealt with readily.

For example where the hypertension is due to chronic pulmonary thromboembolism, the system can be configured and programmed to coordinate the pressure-alleviating diversion of blood with the simultaneous and direct release of a thrombolytic such as tissue plasminogen activator (serine protease), streptokinase, or urokinase and a diuretic to ameliorate the fluid retention and its secondary effects as the result of right heart failure (see, for example, Fischer, L., Benjamin, N.2. Blank, N., Egenlauf, B., Fischer, C., and 8 others 2018. “Right Heart Size and Function Significantly Correlate in Patients with Pulmonary Arterial Hypertension—A Cross-sectional Study,” Respiratory Research 19(1): 216; van der Bruggen, C. E. E., Tedford, R. J., Handoko, M. L., van der Velden, J., and de Man, F. S. 2017. “RV [right ventricle] Pressure Overload: From Hypertrophy to Failure,” Cardiovascular Research 113(12): 1423-1432; Naeije. R.and Badagliacca, R. 2017. “The Overloaded Right Heart and Ventricular Interdependence,” Cardiovascular Research 113(12): 1474-1485; Klaassen, S. H. C., Liu, L. C. Y., Hummel, Y. M., Damman, K., van der Meer, P., and 3 others 2017. “Clinical and Hemodynamic Correlates and Prognostic Value of VE/VCO2 [ventilator equivalent for carbon dioxide] Slope in Patients with Heart Failure with Preserved Ejection Fraction and Pulmonary Hypertension,” Journal of Cardiac Failure 23(11): 777-782; Vonk-Noordegraaf, A., Haddad, F., Chin, K. M., Forfia, P. R., Kawut, S. M. and 8 others 2013. “Right Heart Adaptation to Pulmonary Arterial Hypertension: Physiology and Pathobiology,” Journal of the American College of Cardiology 62(25 Supplement): D22-33), and drugs targeted through an accessory channel of the main pulmonary valve to support the nitric oxide (NO) pathway, endothelin-1 pathway and prostacyclin pathway, and confirm recovery to the set point (see, for example, Vachiery, J. L., Galie. N., Barbera, J. A., Frost. A. E., Ghofrani. H. A., and 8 others 2018. “Initial Combination Therapy with Ambrisentan+Tadalafil on Pulmonary Arterial Hypertension-related Hospitalization in the AMBITION [Ambrisentan and Tadalafil in Patients with Pulmonary Arterial Hypertension] Trial,” Journal of Heart and Lung Transplantation (New York, New York) pii: S1053-2498(18) 31742-X; Galie. N., Barbera, J. A., Frost, A. E., Ghofrani, H. A., Hoeper, M. M., and 14 others 2015. “Initial Use of Ambrisentan Plus Tadalafil in Pulmonary Arterial Hypertension,” New England Journal of Medicine 373(9): 834-844; Velayati. A., Valerio, M. G., Shen, M., Tariq, S., Lanier, G. M., and Aronow. W. S. 2016. “Update on Pulmonary Arterial Hypertension Pharmacotherapy,” Postgraduate Medicine 128(5): 460-473; Anderson, J. R. and Nawarskas, J. J. 2010. “Pharmacotherapeutic Management of Pulmonary Arterial Hypertension,” Cardiology in Review 18(3): 148-162). The system can also record the parameters of and signal crises to the clinic.

A glycogen storage disease (glycogenosis, dextrinosis) can be treated through the automatic release of allopurinol and granulocyte-colony stimulating factor, as well as a diet devised to elevate blood glucose. Automatic response is initiated by the implanted microcontroller upon receipt of an out-of-normal-range input signal from an implanted blood glucose sensor, whereupon the controller actuates the implanted drug reservoir outlet pump or drip stopcock to release the dose of the drug needed to recover to a normal reading. Radioactive substances such as the direct delivery of radioiodine to the thyroid gland is a through radiation shielded line as described in U.S. Pat. No. 11,759,186, entitled Ductus Side-entry Jackets and Prosthetic Disorder Response Systems.

Where the causative factor for an alteration in pulmonary blood pressure is established and singular or few, the implant response system can respond by pipeline targeting the required drug or drugs to the responsible or etiological tissue, structure, or root, nidus, or site, of disease with or without reducing the pulmonary pressure mechanically by diverting the indicated volume of blood to be passed to another vessel, typically the aorta, which large in caliber little effects circulation with the introduction of a relatively slight volume of unoxygenated blood.

Nonpharmacological treatments include pulmonary artery denervation (see, for example, Chen, S. L., Zhang, H., Xie. D. J., Zhang, J., Zhou, L., Rothman, A. M., and Stone, G. W. 2015. “Hemodynamic, Functional, and Clinical Responses to Pulmonary Artery Denervation in Patients with Pulmonary Arterial Hypertension of Different Causes: Phase II Results from the Pulmonary Artery Denervation-1 Study,” Circulation. Cardiovascular Interventions 8(11): e002837), vagal nerve stimulation, of which the implant system is capable (see, for example, Yoshida, K., Saku, K., Kamada, K., Abe, K., Tanaka-Ishikawa, M., and 5 others 2018. “Electrical Vagal Nerve Stimulation Ameliorates Pulmonary Vascular Remodeling and Improves Survival in Rats with Severe Pulmonary Arterial Hypertension,” Journal of the American College of Cardiology. Basic to Translational Science 3(5): 657-671; Ntokou, A. and Greif, D. M. 2018. “Vagal Nerve Stimulation for Pulmonary Hypertension: Some Promise, Some Skepticism,” Journal of the American College of Cardiology. Basic to Translational Science 3(5): 672-674).

However, given that patients can present with causative conditions which combine subtypes of more than one type, for example, for an implant system to automatically respond with any of a large number of drugs targeted to any number of sites challenges the available intracorporeal space for implanting even small drug reservoirs and imposes excessive complexity. In this circumstance, the implant system should be able to adapt to changes in the pulmonary blood pressure as this occurs, with the patient signaled to seek medical help to diagnose and treat the specific cause or causes. Prescriptive regimens that call for the use of non-implantable apparatus, foods, or which call for drugs at a frequency and/or in a volume that even when concentrated and taken with water do not allow implantation are prescribed as adjunctive to that accomplished by the implant system.

When the etiology is elusive, the response will be limited to prescribing medication in conjunction with mechanical diversion of only so much blood from the main pulmonary artery as is necessary to further alleviate the excessive pressure beyond what the medication alone provides, and therewith, attenuate right heart damage, and reduce such symptoms as are apparent. The determination of this shortfall involves delaying the shunting away of any blood from the pulmonary artery until the decline in pressure achieved by the medication is stable, so that if the pressure is reduced to an acceptable level, the valve is not employed. Similarly, where the etiology is recognized but a cure does not yet exist, such as with conditions of genetic origin, combining analgesics and other palliative drugs with mechanical diversion can produce a considerable reduction in the severity of tertiary symptoms. Where a process such as gene therapy is applicable, that treatment or that component in the overall treatment would be administered in the clinic.

An example of the latter are mixed and other connective tissue diseases (see, for example, Correale, M., Ferraretti, A., Monaco, I., Grazioli, D., Di Biase, M., and Brunetti, N. D. 2018. “Endothelin-receptor Antagonists in the Management of Pulmonary Arterial Hypertension: Where Do We Stand?,” Vascular Health and Risk Management 14:253-264; Niklas, K., Niklas, A., Mularek-Kubzdela, T., and Puszczewicz, M. 2018. “Prevalence of Pulmonary Hypertension in Patients with Systemic Sclerosis and Mixed Connective Tissue Disease,” Medicine (Baltimore, Maryland) 97(28): e11437; Cucuruzac, R., Muntean, I., Benedek, I., Mester, A., Rat, N., Mitre, A., Chitu, M., and Benedek, T. 2018. “Right Ventricle Remodeling and Function in Scleroderma Patients,” Online, BioMed Research International 2018:4528148; Argula, R. G., Karwa, A., Lauer, A., Gregg, D., Silver, R. M., and 7 others 2017. “Differences in Right Ventricular Functional Changes during Treatment between Systemic Sclerosis-associated Pulmonary Arterial Hypertension and Idiopathic Pulmonary Arterial Hypertension,” Annals of the American Thoracic Society 14(5): 682-689; Coghlan, J. G., Galiè, N., Barbera, J. A., Frost, A. E., Ghofrani, H. A., and 12 others 2017. “Initial Combination Therapy with Ambrisentan [endothelin hormone blocking drug] and Tadalafil in Connective Tissue Disease-associated Pulmonary Arterial Hypertension (CTD-PAH): Subgroup Analysis from the AMBITION [Ambrisentan and Tadalafil in Patients with Pulmonary Arterial Hypertension] Trial,” Annals of the Rheumatic Diseases 76(7): 1219-1227; de Raaf, M. A., Beekhuijzen, M., Guignabert, C., Vonk Noordegraaf, A., and Bogaard, H. J. 2015. “Endothelin-1 Receptor Antagonists in Fetal Development and Pulmonary Arterial Hypertension,” Reproductive Toxicology (Elmsford, New York) 56:45-51; Mathai, S. C. and Hassoun, P. M. 2012. “Pulmonary Arterial Hypertension in Connective Tissue Diseases,” Heart Failure Clinics 8(3): 413-425; Goldberg, A. 2010. “Pulmonary Arterial Hypertension in Connective Tissue Diseases,” Cardiology in Review 18(2): 85-88; Raeside, D. A., Chalmers, G., Clelland, J., Madhok, R., and Peacock, A. J. 1998. “Pulmonary Artery Pressure Variation in Patients with Connective Tissue Disease: 24 hour Ambulatory Pulmonary Artery Pressure Monitoring,” Thorax 53(10): 857-862), the system satisfies such a palliative level of therapy through combining the targeted release of drugs into the main pulmonary artery with the diversion of the main pulmonary artery blood in step with the pressure variance. Only an implant system is able to instantly detect and respond to changes in pulmonary arterial pressure, whether due to an underlying disease, such as mixed connective tissue disease, exercise, or changes in body position.

The implant system relieves the excess pressure within and releases drugs into the main pulmonary artery through the same valve or valves, diverting blood through the mainline and instantly releasing the prescribed drugs through the accessory channel or channels, or sidelines of that same pulmonary valve. At the same time, medication to treat the other symptoms is instantly pipeline targeted to ductus side-entry jackets or nonjacketing side-entry connectors at those other sites. The microcontroller, or in a more elaborate system, the microprocessor, controls the volume of blood to be diverted, generally into the aorta, in coordination with the release of corresponding doses of the drug or drugs needed.

Where the cause is singular or few and identified in a prenate or neonate, the immediacy with which an implanted system can respond to the symptoms may be sufficient to avert the development of pulmonary hypertension with consequent heart injury. In a patient of any age, to require continuous monitoring of the pulmonary arterial blood pressure alone and respond with diversion and coordinated medication into the pulmonary artery falls well within the capabilities of an implant system.

Continuous or dynamic tracking of variation in the pressure within the main pulmonary artery-seen in connective tissue disorders as cited above, as well as in response to exercise or changes in body position—is made possible by tiny sensors attached to the artery by nonjacketing side-entry connectors as well as incorporated into the valve body (Mizumi, S., Goda, A., Takeuchi, K., Kikuchi, H., Inami, T., Soejima, K., and Satoh, T. 2018. “Effects of Body Position during Cardiopulmonary Exercise Testing with Right Heart Catheterization,” Physiological Reports 6(23): e13945; Correale, M., Tricarico, L., Ferraretti, A., Monaco, I., Concilio, M., and 4 others 2017. “Cardiopulmonary Exercise Test Predicts Right Heart Catheterization,” European Journal of Clinical Investigation 47(12); Rhinehart, J. D., Schober, K. E., Scansen, B. A., Yildiz, V., and Bonagura, J. D. 2017. Effect of Body “Position, Exercise, and Sedation on Estimation of Pulmonary Artery Pressure in Dogs with Degenerative Atrioventricular Valve Disease,” Journal of Veterinary Internal Medicine 31(6): 1611-1621; Chen, L. Y., Tee, B. C., Chortos, A. L., Schwartz, G., Tse, V., and 4 others 2014. “Continuous Wireless Pressure Monitoring and Mapping with Ultra-small Passive Sensors for Health Monitoring and Critical Care,” Nature Communications 5:5028; Tolle, J. J., Waxman, A. B., Van Horn, T. L., Pappagianopoulos, P. P., and Systrom, D. M. 2008. “Exercise-induced Pulmonary Arterial Hypertension,” Circulation 118(21): 2183-2189; Raeside, D. A., Chalmers, G., Clelland, J., Madhok, R., and Peacock, A. J. 1998, Op. cit.).

2b(2)(h)(h4). The ‘Tuned’ Shunt in the Relief of Localized Vascular Hypertension

The relief of a localized hypertension is not a cure, which usually relates to obstruction to flow posed by the supplied organ, and is intended to lessen the injury the condition causes, usually pending transplantation of the obstructive organ. Removal of the cause is the preferred response; however, when intractable, a means of amelioration can reduce the pathology that would otherwise result. A “runed” shunt such as shown in FIG. 22F makes it possible to tap off a sufficient volume of flow through the hypertensive vessel and move it into one or more other vessels to dissipate the hypertension and prevent its consequences.

One such problem is valvular disease due to excessive output force of the right ventricle which to alleviate with the aid of a tuned shunt should considerably reduce the extent of surgical trauma (see, for example, Bhattacharya, P. T., Shams, P., and Ellison, M. B. 2024. “Right Ventricular Hypertrophy,” Online, Treasure Island, Florida: StatPearls Publishing Co.; Monteagudo-Vela, M., Tindale, A, Monguió-Santin, E., Reyes-Copa, G., and Panoulas, V. 2023. “Right Ventricular Failure: Current Strategies and Future Development,” Online, Frontiers in Cardiovascular Medicine (Lausanne, Switzerland) 10:998382; Borgdorff, M. A., Dickinson, M. G., Berger, R. M., and Bartelds, B. 2015. “Right Ventricular Failure Due to Chronic Pressure Load: What Have We Learned in Animal Models Since the NIH Working Group Statement?, Online, Heart Fail Reviews (Norwell, Massachusetts) 20(4): 475-491; Naeije, R., Brimioulle, S., and Dewachter, L. 2014. “Biomechanics of the Right Ventricle in Health and Disease (2013 Grover Conference Series),” Online, Pulmonary Circulation (Hoboken, New Jersey) 4(3): 395-406; Serraf, A., Nakamura, T., Lacour-Gayet, F., Piot, D., Bruniaux, J., and 4 others 1996. “Surgical Approaches for Double-outlet Rght ventricle or Transposition of the Great Arteries Associated with Straddling Atrioventricular Valves,” Online, Journal of Thoracic and Cardiovascular Surgery (St. Louis, Missouri) 111(3): 527-535).

As in autoimmune chronic pancreatitis, which can lead to endocrine and exocrine insufficiency, localized arterial hypertension is secondary to resistance posed by the supplied end-organ or tissue (see, for example, Hart, P. A., Zen, Y., and Chari, S. T. 2015. “Recent Advances in Autoimmune Pancreatitis,” Gastroenterology 149(1): 39-51; Chintanaboina, J., Yang, Z., and Mathew, A. 2015. “Autoimmune Pancreatitis: A Diagnostic Challenge for the Clinician,” Southern Medical Journal 108(9): 579-589; Sah, R. P. and Chari, S. T. 2012. “Autoimmune Pancreatitis: An Update on Classification, Diagnosis, Natural History and Management,” Current Gastroenterology Reports 14(2): 95-105; Korot'ko, G. G., Faustov, L. A., Sychova, N. L., Sychov, V. A., and Popova, E. V. 2005. “Local Arterial Hypertension in the Pathogenesis of Chronic Pancreatitis,” (in Russian with English abstract at Pubmed), Eksperimental'naia i Klinicheskaia Gastroenterologiia [Experimental and Clinical Gastroenterology (2): 49-53; Korot'ko, G. G., Faustov, L. A., Sycheva, N. L., and Sychev, V. A. 2002. “Local Arterial Hypertension in the Pathogenesis of Ulcers and Chronic Pancreatitis,” (in Russian with English abstract at Pubmed), Eksperimental'naia i Klinicheskaia Gastroenterologiia (Experimental and Clinical Gastroenterology)(6): 48, 113), or resistance to flow imposed by an obstructed or constricted arterial wall.

Where the primary cause is refractory to correction or the treatment best withheld from the patient but endothelial dysfunction would not preclude the release of nitric oxide (see, for example, Gee, L. C. and Ahluwalia, A. 2016. “Dietary Nitrate Lowers Blood Pressure: Epidemiological, Pre-clinical Experimental and Clinical Trial Evidence,” Current Hypertension Reports 18(2): 17; Shiekh, G. A., Ayub, T., Khan, S. N., Dar, R., and Andrabi, K. I. 2011. “Reduced Nitrate Level in Individuals with Hypertension and Diabetes,” Journal of Cardiovascular Disease Research 2(3): 172-176), reduction in localized arterial hypertension is achieved through the coordinated release of pipeline targeted medication leading to the release of nitric oxide, such as isosorbide dinitrate, glyceryl trinitrate, or nitroglycerin, and if necessary, further reduction in pressure through diversion of a fraction, limited to that essential, of the flow to a large artery.

2b(2)(i). Aneurysm Bypass Conduit and Local Blood Pressure Relief-Passive One-Way and Pressure Feedback-Controlled Diversion

Aneurysm bypass is an exigent expedient intended to avoid the possibility of a rupture pending replacement of the defective artery such as described above in section 2b(2)(c)(c2), entitled Automatic Insertion into a Ductus of an Inline Coupling Jacket-secured Bridging, or Tie-line and Its Replacement if Necessary. Replacement prostheses such as confluence conduits, aneurysm bypass conduits, and bridges or tie-lines are made with an elastic and expandable fabric with high tensile strength for resistance to bursting or rupture described below in section 2b(2)(f), entitled Expandable Structure to Accommodate Growth and Self-adjust in Size in Vascular Segment Prostheses, Lines, and Confluence Conduits Placed in Children. This is especially important for infants, who are spared the need for painful revision following growth.

Blood pressure relief with the aid of diversion jacket and servovalves is addressed above in sections (c). Ischemia-free Aortic Repair, to Include Coarctation and Aneurysm, and Targeted Drug Delivery, (e). Vascular Pressure Relief, as well as in this section. With an aneurysm of the aortic arch due to or aggravated by elevated blood pressure, a blood diverting servovalve can be used to divert a proportion of blood sufficient to bring the pressure down to within the safe range.

Such valves always incorporate at least one accessory channel through which hypertension counteractive medication such as angiotensin converting enzyme inhibitors, angiotensin II receptor blockers, alpha blockers, beta-blockers, various combinations of alpha and beta blockers, calcium channel blockers, alpha-2 receptor agonists, diuretics, and vasodilators can be directly pipeline targeted to little short of the aortic root, thus ameliorating the elevated pressure systemically and not just locally while averting the common adverse side effects of dizziness, bradycardia, presyncope lightheadedness, syncope, vertigo, and cephalalgia.

2b(2)(i). Vascular Servochokes

Sphincteric extraluminal, or perivascular, as opposed to endoluminal stopper plate-type flow-adjusting and stopping valves overlap in function but without endoluminal (intraluminal) of endovascular (intravascular) parts. Useful when the lumen would best be avoided, these are addressed below in section 2b(2)(j)(j1), entitled Perivascular Sphincter-type Flow Adjusting and Stopping Valves. That is, from a functional standpoint, these are substantially interchangeable, the perivascular, or external sphincteric type allowing a presence within the lumen to be avoided. Endoluminal servo chokevalves, are addressed below in sections 2b(2)(j)(j2), entitled Intraluminal Flow-adjusting Servo Chokevalves, or Servochokes, and a mechanical stopper in section 2b(2)(j)(j3), entitled Rectal Stopper.

A servochoke such as that shown in FIG. 14A is a type of servovalve driven by a servomotor such as that shown in FIG. 9 used to affect the local up- and downstream blood pressure along a vessel for a purpose such as increasing the uptake of a drug or nanoparticulate in the stream as in endothelial drumming and to introduce a supraparamagnetic (or superparamagnetic) iron oxide nanoparticle trap. That the driver could also be lead screw driven is considered obvious. It differs from a servovalve of the kind shown in FIG. 10A in lacking a diversion chute 18 to controllably tap off a fraction of the flow for redirection into another, usually larger, vessel of the same kind, being instead provided with an obturator 44.

Any of these can be used with ductus side-entry diversion type vascular valves and servovalves to adjust local blood pressure. Extraluminal, or sphincteric-type valves, arranged and actuated in linear sequence to simulate peristalsis, are relevant to vascular valves and servovalves as applicable to conditions involving the digestive tract, frequently encountered in comorbid disease, suited to treatment by an implanted automatic hierarchical control system. Chokevalves must be continuously variable in controlling the flow through area, and are therefore servo chokevalves, or servochokes, which are servomotor, not solenoid driven.

Unlike servovalves, which tap off a continuously fraction of flow into either of two passageways, servochokes have only one outlet. For this reason, while highly unlikely, servochokes pose a potential risk for complete closure following a malfunction, and therewith, ischemia in the supply territory. An ability for complete closure with the arrest and backup of flow, or blood stoppage, is exclusively a danger and never advantageous, so that servochokes are made incapable of failing thus. In contrast, servovalves almost must always be able to redirect flow from one fluid line in to another entirely as well as portionally, and a failure to cut off all flow from the original path would redirect flow into the unintended outlet, never likely to pose a medical emergency.

To assure servochoke displacements are as commanded and incapable of deviating under higher pressures, the armature is notched at intervals along its length and a detent consisting of a prominence along its bore, or channel, provided to hold the armature in place at the position commanded, a relatively stronger power surge commanded by the implanted controller, having been signaled the deviation from the instant set point, then used to move it. Servochokes intended for positioning along lines subject to higher pressures are likewise provided with this means for assuring positional stabilization and preventing the possibility of a complete closure causing ischemia. Servovalves are similarly designed; however, closure a necessary setting for the complete redirection of flow when intended, these do not, as do servochokes, incorporate a notch to eliminate the possibility of complete closure.

Independently of motile function, in treating the gut, that the accessory channels (sidelines, druglines) incorporated into all of these as well as basic side-entry valves can directly target drugs to any section along the digestive tract is of central benefit in the treatment of comorbid disease, the gut seldom uninvolved. Blood pressure relief servovalves usually accompany choke valves used to stabilize the blood pressure by diverting, or ‘bleeding off’ just so much blood as reduces the pressure to within the normal range, thus eliminating the elevated pressure that alone or in combination with an infection, connective tissue disorder, or vasculitis, for example, caused the elastic limit of the arterial wall to be exceeded, thus inducing the structural failure.

Reduction in local blood pressure through the use of diversion, and the increase in local blood pressure through the use of obstructive devices are expedients to be used only when the primary cause for the deviation from normal pressure is inoperable or the condition of the patient recommends against operative intervention. Diversion servovalves can be continuously adjusted to accomplish the function of alternative type valves used to alter the local blood pressure without at the same time raising the upstream or retrograde pressure. Using these, the reduction in pressure is accomplished by diverting a proportion of the flow to another vessel, usually one larger where the increase in volume has little if any effect. When the largest vessels, pulmonary artery, portal vein, are affected, diversion is to a plurality of neighboring vessels.

However, for certain purposes, such as to increase drug uptake retrograde to the valve, it is desired that the pressure ahead of the valve be increased. FIGS. 2, 5, 7, 8, and 10A show side-entry jacket-enclosed or periductal type valves with an intravascular flow diversion chute, and FIG. 11 in U.S. Pat. No. 11,759,186 shows a periductal or sphincter type valve. Almost all valves and servovalves incorporate at least one accessory channel (secondary channel, sideline) to allow the direct pipeline targeting of drugs to and through the jacket.

Used to adjust the pressure by constriction rather than diversion, servochokes intravascular and periductal are more likely to omit a mainline. While a conventional ductus side-entry jacket with a lip, effectively a small chute, at the opening leading into the side connector can be used to divert blood, and the lip can be lengthened into a kind of nonadjustable chute, a controllable chute can be used as an in-situ trimmer that can be extended and retracted to adjust diversion to the pressure and velocity of the blood optimal for the specific application.

Whether to relieve a diseased stretch along a vessel, an aneurysm, or to expedite the healing of a lesion or a segment operated upon, diversion valve and adaptive servovalve-jackets can relieve, and adaptive choke servovalve-jackets, or electrically adjustable sphincters, increase blood pressure through the substrate vessel. The entire flow or a fraction thereof can be bypassed around a diseased segment or aneurysm in the substrate vessel, and local deviations from normal blood pressure leveled by tapping off a portion of flow and passing it into a larger like vessel.

In such use, the reduction in blood pressure is the end purpose in itself; however, reducing the blood pressure increases its velocity reducing uptake of the drug through the affected segment, while increasing the pressure reduces the velocity increases uptake, allowing a vessel to be differentially medicated along its length. Drugs may be directly pipe-targeted into the substrate native lumen through a separate upstream side-entry valve jacket or through a drugline, 8′ inserted into a valve accessory channel such as that shown in FIGS. 2, 5, 7, 8, and 10A or through the integral drugline 8 coursing through the flow diversion chute 18 along the bottom of the jacket.

That increasing and decreasing the extension into the native lumen of the obstruction plate (choke plate, flat obstruction plate, obstructor, obturator) obstructor, part number 43 in FIG. 14A or the flow diversion chute 18 in a vascular valve or servovalve adjusts the pressure in the obstructed or the undiverted fraction allows the more controlled infusion of a drug through an integral drugline 8 or 8′ to be coordinated with its uptake along the vessel. Consistent with Bernoulli's principle, positioning a separate servochoke, or choke servovalve upstream from a diversion valve to partially obstruct and thus increase the velocity of flow can be used to augment the reduction in blood pressure due to diversion at the vascular valve or servovalve.

Whether to relieve pressure to expedite healing or increase it to increase drug uptake, to adjust diversion or choke servovalve manually is tied to the clinic. The value in pressurizing and thus decelerating the flow at levels for increased uptake is increased when the drug is carried by magnetically susceptible nanoparticles and the diversion or servovalve jacket used incorporates a surrounding layer of neodymium iron boron or tiny electromagnets of which the fields are oriented perpendicular the central axis of the sending artery. Counterintuitively, such coordination is more reliably accomplished not in the clinic but rather by an implanted microcontroller of which the prescription-program coordinates the release of drugs with adjustments in the extension of the obstructor in each jacket along the vessel.

The drugs are injected into subdermal reservoirs implanted in the pectoral region, and the microcontroller effects release by control of the small pump at the outlet of the reservoir. Accordingly, this more refined control is better accomplished in an ambulatory automatic disorder response system wherein feedback form implanted sensors deliver information to the microcontroller which responds by coordinating the release of drugs with the variations in blood pressure along the vessel.

The concept is also applicable to the gut. Manually controlled as endoluminal valves or motor-driven as vascular servovalves, or servochokes, chute jackets can be fully or partially extended into the native lumen to effectuate the partial or complete diversion of flow. When partially extended, diversion jackets release a controlled fraction of the flow through the ductus, reducing the pressure, the diverted portion returned downstream to the same or to another artery or vein. Intravascular diversion servovalves and sphincteric or perivascular servo chokevalves can be automatically adjusted by the implant microprocessor of a prosthetic disorder response system in accordance with the prescription-program.

Adjustment thus is generally coordinated with the release into the ductus of medication through an accessory channel, which if magnetically carried, can be momentarily detained the diversion servovalve or servo chokevalve when including a permanent or electromagnet layer. The increase in pressure and reduction in velocity of the blood or urine upstream to the valve or choke allows more of the drug to be drawn against or through the endothelium or urothelium. As stated above in section (b1). Endothelial Drumming, control thus is continuously variable. Where proper repair or transplantation must be deferred, a servo diversion jacket or servochoke is used in lieu of pulmonary artery banding, the latter adjustable from outside the body but not readily or away from the clinic.

A side-entry vascular valve or servovalve with a diversion chute 18 but not a diversion outflow line through a sidestem 19 is effectively an inappropriate choke-valve which obstructs rather than diverts flow-through. However, if fully retracted, diversion chute 18 would cut off flow-through completely to eliminate any diversion. Such would represent a medical emergency anywhere in the vascular tree or along a ureter. In contrast, obstructors in other shapes such as domes or cones are incapable of completely obstructing flow in the event of a malfunction, and these affect the flow lines differently than a chute as may favor an eccentric or asymmetrical lesion, for example. Where diversion is uninvolved, these choke valve-jackets, which may be used in various combinations at intervals along the ductus, are used to adjust the local blood pressure.

Intravascular and periductal servochokes can adjust the local blood pressure, intentionally increasing or reducing the forces applied along a vulnerable segment, and a choke jacket with a magnetic component can take advantage of the reduction in velocity or flowrate of a passing ferrofluid to extract drug-carrying nanoparticles, and a choke jacket with radiation shielding can do so where the nanoparticles are of low to moderate radioactivity. Unshown, a servochoke with a cone rather than a flat plate obturator (obstruction plate, obturator) as shown in FIG. 14A could be used to partially choke flow by measured extension and retraction into and out of the native lumen either under manual control with a knob on the surface port to control a Bowden push/pull cable, or if automated, by a linear servomotor-driven obturator.

Whereas a choke jacket can use a chute, dome, or cone, a diversion jacket requires a chute. A push/pull solenoid is unsuitable for fractional diversion or fractional obstruction but suitable in an all or nothing flow diversion jacket such as is needed in an organ transplant or carotid endarterectomy. In the repair of a great vessel discordance, diversion of blood must be in certain proportion, requiring manual or servomotor adjustment. When both choke and diversion functions are required, a vascular valve with a flow diversion chute and connection to a bloodline, or mainline, is needed. Where diversion is unnecessary, a choke valve without connection to a mainline but with at least one accessory channel to insert a drugline 8′ in FIG. 14A allows the release of an intermittent drop of heparin, for example

The entry of blood so that clot accumulates in the space above the choke is less pertinent in choke valves with chokes of different shape than a chute but can be dealt with the same manner as with a chute type choke. Where the number of jackets must be kept to a minimum and a choke valve with a potential need for an in- and/or diversion outflow, hence, a mainline exists, the choke of obstructor in the one jacket must be a flow diversion chute. Blood is prevented from clotting in the space above the chute when not fully retracted by capping off the side connector and using the water jacket as an accessory channel to deliver an anticoagulant in a slow drip.

Should the need to use the chute type choke valve to divert blood, if not attached from the outset, a mainline from the surface port is attached to the side connector inserted. Where the urine contains a pathogen, the drip contains an antimicrobial; if other nocuous matter, then a suitable counteractant. With respect to a choke valve-jacket used in the bloodstream (but not along a ureter where flow past the outlet is not continual) when an unused mainline is provided from the outset, preventing the entry of blood into the line can be accomplished by filling the line with water or a liquid such as Ringer's, or Ringer's lactate solution (Hartmann's solution, sodium lactate solution). If appropriate, control of paired valves is coordinated by the microcontroller implant, which can also control the direct delivery to the valves of drugs through their accessory channels.

Such valves provide the ability to lower the blood pressure through a circumscribed segment of a vessel which has been injured, repaired and healing, contains a stent, inferior vena cava filter, aneurysm, and so on, mechanically, without the need to expose the entire circulatory system to an antihypertensive. The chute has feathered, or progressively thinner edges which elastic, unfold or fan out as its sides move toward the center of the lumen and fold as the center is passed. Ductus side-entry blood pressure adjustment jackets may omit the mainline, providing a side connector for attachment of one or more accessory channels for the direct targeting to the treatment site of drugs and passage of cabled devices from a port at the body surface.

However, the free end of the side connector should include a cap to allow for the insertion of a mainline to the surface port should the need arise. In those using a side-entry diversion chute, the accessory channels feed as do side-entry diversion jackets generally, through a channel running along the bottom center of the chute. Of the many classes of antihypertensive drugs, none is free of potentially serious side effects. For this reason, the direct targeting of drugs to the segment intended averts complications. When uptake moving through the target segment, which may seek to medicate the intima or the supply territory might prove harmful to healthy tissue, a drug for which there is a reversal agent is chosen and a second jacket positioned at the terminus of the segment to be treated for delivery of the reversal agent. With or without a mainlines, choke valves may also be referred to as pressure adjustment valves.

FIG. 14A shows a side-entry servochoke valve jacket which can be used to increase, and FIGS. 7 and 8 side-entry vascular flow diversion valves which can be used to decrease the downstream blood pressure (Tijsseling, A. S. and Anderson, A. 2008. “Thomas Young's Research on Fluid Transients: 200 Years On,” in Hunt, S., (ed.), Proceedings of the 10th International Conference on Pressure Surges, Edinburgh, Scotland, available online at http://www.win.tue.nl/˜atijssel/pdf_files/CASA-08-16.pdf; Giaquinta, A. R. 1971. “The Historical Development of the Engineering Analysis of Blood Flow,” La Houille Blanche [hydroelectric power] 26(4), 327-341; Young, T. 1808. “Hydraulic Investigations, Subservient to an Intended Croonian Lecture on the Motion of the Blood,” Paper XIII in Philosophical Transactions of the Royal Society of London 98:164-186; available at https://archive.org/details/philtrans06120460); c. thru f. shows the use of paired choke with excurrent, and g. thru j., the use of paired choke with incurrent diversion jackets.

Direct diversion from the inferior vena cava to the abdominal aorta reduces the blood pressure beneath the level of the aortic jacket. In each case, the jackets are remotely controllable at the surface port or by radio remote control, and each is provided with at least one accessory channel to target drugs through the jacket and into the bloodstream. Each jacket can incorporate a physiological sensor, and an above-skin body surface port opening allows the passing of cabled devices, such as a fine fiberscope, laser, or intravascular ultrasound probe, directly to the site of treatment.

### Means for Determining the Optimal Blood Pressure for the Endothelial Uptake of Disorder-Pertinent Drugs and Sufficiency of Responsive Secretory Function

A means for continuously varying the local blood pressure within a vessel would allow a detailed quantitative determination of the degree of endothelial uptake and secretory function responsive to the blood pressure and therefore the optimal pressure for the release of diagnostic and therapeutic substances. FIG. 10A shows a vascular servovalve which combined with a vascular servochoke, can be positioned to modulate the local blood pressure above and below it.

With the sidestem 19 closed off to outflow by a servochoke such as that shown in FIG. 14A with fully extended obturator 43 positioned immediately behind the sidestem 19, blood flow through the valve or servovalve lumen caudad, and the curved tip of the chute directed upward, the combination acts much as the servochoke shown in FIG. 14A, the blood pressure increased above the chute and lowered beneath it in proportion to the extension into the lumen of the diversion chute. With the sidestem patent to tap off flow into a larger vessel, such adjustments in pressure are much reduced.

Such a coordinated servovalve/servochoke pair makes possible adjustment in blood pressure from zero to a maximum with or without the use of positive or negative inotropes. Coordinated control of such a pair to allow continuous variability in the intraluminal blood pressure is programmed for automatic execution by the implanted disorder response system master microcontroller. That is, the disorder response system executes algorithms that control servovalve-servochoke coordinated adjustments in blood pressure and quantifies and records the resultant values for responsive uptake of analytes such as drugs, cholesterol, or calcium, for example, or the release by the endothelium of substances responsive to the appropriate stimulants.

In therapeutic use, the controller maintains the local blood pressure at the level for optimal uptake and releases the drug or sustains the level of endothelial substance secretion responsive to the detection of the adverse symptom by deployed sensors. If to remain as fully implanted, the release of analytes is through druglines into the servovalve, and measurements are taken just downstream to it. Placement of such a pair necessarily invasive, clinical use is limited to applications where open surgery had been essential anyway or where the long-term treatment of a chronic disorder is sought.

Provided the analytes involved in the disorder presented by the patient are few, full implantation is possible. Where disease is multiply morbid, transmission to a blood gas console analyzer while the patient remains open is possible. Such blood gas analyzers can measure the levels of numerous substances to include carboxyhemoglobin, methemoglobin hemoglobin, lactate, electrolytes, and oxyhemoglobin. Otherwise, use is limited to physiological studies.

The continuous monitoring of blood pressure is by a thin film pressure gauge inside the servovalve. Analysis of blood concentration of the analyte under study is by extraluminal perivascular pickups which combine features of oximetry and colorimetry with the use of contrast agents as necessary to quantify the uptake of substances or the secretion of others by the endothelium. The concentration quantifying sensors are intraluminal and at a regular level downstream to the servovalve and servochoke.

The prescription-program can apply conversion factors to adjust readings for various deviations from the actual point of pickup or other physical circumstances. A definite challenge to the implementation of this method is that some vessels most consequential for the acquisition of such data, such as the coronaries and the carotids, must not be manipulated thus in a living patient, and endothelia in different parts of the body, such as endocardial, coronary, aortic, arterial, and venous differ, so that data gathered at any one site is unlikely to prove applicable to any other.

Relevant here is that the endothelia of a subject who expired while on extended extracorporeal membrane oxygenation life support to be sustained past brain death with associated sustainment support such endocrine and nutritional as would be available in a deceased donor sustainment center can remain well preserved for a period of several weeks. Accordingly, the deceased donor sustainment centers addressed in section 2a, entitled Background of the Invention. General, in the subsection thereof of like title can also serve as a source of test subjects.

Research on endothelial diversity and related subjects is advanced (see, for example, Abaci, H. E. and Esch, M. B. 2024. “Organ-specific Vasculature-on-a-chip Systems,” Online, [American Institute of Physics] Biomicrofluidics (Melville, New York) 18(3): 030401; Iovino, L., Krenning, G., and Hadland, B. 2024. “Editorial: Unconventional Roles of Endothelial Cells,” Online, Frontiers in Cell and Developmental Biology (Lausanne, Switzerland) 12:1439419; Jerka, D., Bonowicz, K., Piekarska, K., Gokyer, S., Derici, U. S., and 7 others 2024. “Unraveling Endothelial Cell Migration: Insights into Fundamental Forces, Inflammation, Biomaterial Applications, and Tissue Regeneration Strategies,” Online, American Chemical Society Applied Bio Materials (Washington, DC) 7(4): 2054-2069; Lim, X. R. and Harraz, O. F. 2024. “Mechanosensing by Vascular Endothelium,” Online, Annual Review of Physiology (Palo Alto, California) 86:71-97; Meijer, E. M., van Dijk, C. G. M., Giles, R., Gijsen, K., Chrifi, I., Verhaar, M. C., and Cheng, C. 2024. “Induction of Fenestrae in Human Induced Pluripotent Stem Cell-derived Endothelial Cells for Disease Modeling,” Tissue Engineering. Part A (New Rochelle, New York) 30(3-4): 168-180; Yang, D. R., Wang, M. Y., Zhang, C. L., and Wang. Y. 2024. “Endothelial Dysfunction in Vascular Ccomplications of Diabetes: A Comprehensive Review of Mechanisms and Implications,” Online, Frontiers in Endocrinology (Lausanne, Switzerland) 15:1359255; Becker, L. M., Chen, S. H., Rodor. J., de Rooij, L. P. M. H., Baker, A. H., and Carmeliet, P. 2023. “Deciphering Endothelial Heterogeneity in Health and Disease at Single-cell Resolution: Progress and Perspectives,” Online, Cardiovascular Research (Oxford, England) 119(1): 6-27; Palomo, M., Moreno-Castaño, A. B., Salas, M. Q., Escribano-Serrat, S., Rovira. M., and 9 others 2023. “Endothelial Activation and Damage as a Common Pathological Substrate in Different Pathologies and Cell Therapy Complications,” Online, Frontiers in Medicine (Lausanne, Switzerland) 10:1285898; Trimm, E. and Red-Horse, K. 2023. “Vascular Endothelial Cell Development and Diversity,” Online, Nature Reviews. Cardiology (London, England) 20(3): 197-210; Wakabayashi, T. and Naito, H. 2023. “Cellular Heterogeneity and Stem Cells of Vascular Endothelial Cells in Blood Vessel Formation and Homeostasis: Insights from Single-cell RNA Sequencing,” Online, Frontiers in Cell and Developmental Biology (Lausanne, Switzerland) 11:1146399; Hennigs, J. K., Matuszcak, C., Trepel, M., and Korbelin, J. 2021. “Vascular Endothelial Cells: Heterogeneity and Targeting Approaches,” Online, MDPI [Multidisciplinary Digital Publishing Institute] Cells (Basel, Switzerland) 10(10): 2712; Paik, D. T., Tian, L., Williams, I. M., Rhee, S., Zhang, H., and 5 others 2020. “Single- cell RNA Sequencing Unveils Unique Transcriptomic Signatures of Organ-specific Endothelial Cells,” Circulation (Hagerstown, Maryland) 142(19): 1848-1862; Przysinda, A., Feng. W., and Li, G. 2020. “Diversity of Organism-wide and Organ-specific Endothelial Cells,” Online, Current Cardiology Reports (Philadelphia, Pennsylvania) 22(4): 19; Marcu, R., Choi, Y. J., Xue. J., Fortin, C. L., Wang, Y., and 10 others 2018. “Human Organ-specific Endothelial Cell Heterogeneity,” Online, iScience (Cambridge, Massachusetts) 4:20-35; Potente, M. and Makinen. T. 2017. “Vascular Heterogeneity and Specialization in Development and Disease,” Nat Reviews. Molecular Cell Biology (London, England) 18(8): 477-494).

Both a diversion servovalve with and on without an outflow line or mainline attached can serve as a servochoke. Local adjustment in blood pressure is by adjusting the extension of the chute into the native lumen thus modulating flow past the chute, either to a lower blood pressure by placing the flow-through side where the less pressurized, hence, higher velocity flow passes to face the lesion, reducing the stress on a diseased or operated upon segment, such as following the repair of an aneurysm, thus presenting less pressure, expediting healing, or by facing away from the affected side of the vessel, thus presenting greater pressure to the vessel in order to strengthen the tissue.

In a choke valve with no outflow mainline attached, blood is prevented from outflow through the mainline space above the chute by capping off the side connector. To prevent blood that enters the trepan end-piece in the side connector, an anti-coagulant, such as a low molecular weight heparin, is sent as a slow drip through the water jacket now following its use to place the jacket, used as an accessory channel. The heparin is injected into a flat reservoir placed in the pectoral region and is released to flow through the water jacket by actuating the pump at the reservoir outlet by the timing module or microcontroller implant. Servo chokevalves can be used, for example, to adjust flow through the main pulmonary or portal artery remotely. Banding has been improved to the extent of design to allow adjustment percutaneously, however, reentry thus is required to move from one fixed setting to the next, so that the response remains independent of changes in posture, exertion, and the pulmonary blood pressure, hence, incapable of dynamic response.

With respect to choke valves regardless of the type of obstructor, a reduction in pressure can also be used to facilitate the uptake of superparamagnetic iron oxide nanoparticles carrying a drug or drugs, and/or to allow magnetic hyperthermia or radiotherapeutic internal radiation with the aid of an external radiofrequency alternating magnetic field generator. Noncritical reductions in blood pressure can be accomplished with nonadjustable ductus side-entry diversion or choke jackets. Precise adjustments require a ductus side-entry diversion or choke jacket with linear motor-driven chute, dome, or cone-shaped obstructor.

With a chute type choke or obstructor, the blood pressure is continuously variable in three ways, whereas using chokes of different conformation, there are two. The first, pertaining to the chute choke, which viewed along its long central axis is not radially symmetrical but rather everted upward at its distal or adluminal tip, is by setting the range, or gross adjustment. With a chute, that most often applicable, rather than a dome or cone configured choke, this is accomplished by positioning the jacket either upright, so that the raised tip of the chute faces upward against the oncoming flow, or inverted, so that the curvature matches the flow in direction to present less resistance to the downflow.

That by providing a slot along which to rotate the chute accessory channel connector a jacket with a rotatable chute can be made is considered obvious but would entail disproportionately greater expense to manufacture than the benefit to be gained. The second way applies to choke jackets regardless of the type choke as to chute, dome, or cone used. Fine adjustment of the choke jacket is by setting the degree of choke extension into the lumen. In a manual system, this done by the clinician with a calibrated knob on the surface port which moves the chute or otherwise configured choke by means of the push-pull control cable arrangement shown in FIG. 30. Where more than one choke jacket is used, each may incorporate a differently configured choke, but each requires a control knob on the surface port.

The setting or settings are maintained against accidental or deliberate change by a keyed cap separate from the dome cap over the entire face of the port, which must remain accessible should the need for a drug or drugs eventuate. Manual adjustment is, however, static, limited to the clinic, and requires knobs on the port. In contrast, automatic control is by an implant microcontroller, which to adjust the extension into the lumen of a choke, varies the current to the continuously variable linear motor that drives the choke or chokes. Such control can respond to feedback from sensors within the jackets. The controller response can consist not only of adjustment in extension of the chokes but the delivery of drugs to each jacket through the jacket sideline or sidelines (druglines). Side-entry diversion and choke valve-jackets can incorporate any or all of the features specified for ductus side-entry jackets.

U.S. Pat. No. 11,759,186 shows ductus side-entry jackets magnetized to draw magnetically susceptible spherules or tiny elliptical bands, with or without drug coating infixed within the ductus adventitia radially outward to prevent the ductus from stenosing. The spherules, referred to as miniballs, are placed from within the lumen by a transluminal aeroballistic discharge barrel assembly. Stays are elongated ellipses with a sharp leading edge and tiny compared to the corset stiffeners.

Stays are placed with ordinary surgical pliers or a stapler-like insertion device passed through a small or ‘keyhole’ incision for endoscopic extravascular access into the adventitia. These implants and the means used to implant them are explained and illustrated in U.S. Pat. No. 11,389,171, entitled Integrated System for the Infixion and Retrieval of Implants. Miniballs are inserted aeroballistically from within the lumen, so that when a jacket must be placed and no other transluminal procedure is required, stays, which are infixed from outside the ductus, are used. When susceptibility to a magnetic field as in a perivascular stent is not need, polymeric stays can be used.

As shown in FIG. 1, provided the vacuum and drug-to-adventitia or drug-to-fibrosa vacuum and drug distribution mantle is made of a nonmagnetic material such as a low resorption polypropylene (PP) or polyetheretherketone (PEEK) or a substantially nonmagnetic stainless steel, that is, an austenitic higher nickel content stainless steel having a maximum magnetic permeability of 1.004, a single ductus side-entry diversion jacket can incorporate both the vacuum and drug mantle and a permanent or electromagnetic layer to draw magnetized particles infused through a side-entry jacket into the organ or tissue blood supply and dispersed therein. Generally, a permanent magnet layer is suitable for drawing the magnetized particulate or particulates up against the endothelial or urothelial lining, for example, and holding these there, whereas an electromagnet layer is needed to draw the particulate or particulates into the intima or comparable tissue.

The use of magnetized infusates, usually superparamagnetic iron oxide nanoparticles in a ferrofluid delivered through the body surface port and detention of the particulate along the level along the ductus of the jacket or side-entry connector is addressed in U.S. Pat. No. 11,389,171, entitled Integrated System for the Infixion and Retrieval of Implants and 11,759,186, entitled Ductus Side-entry Jackets and Prosthetic Disorder Response Systems. The capability added to that of nonvalvular ductus side-entry jackets and nonjacketing side-entry connectors with such a magnetic layer by ductus side-entry diversion jacket vascular valves and servochokes is that the chute or choke obstructor can be extended into the native lumen thereby impeding blood or urine flow, the reduction in the velocity of flow supporting better uptake of the magnetized particulate.

Another application for ductus side-entry and diversion jackets with layer magnetized to project the field adluminally is as an extraluminal stent: magnetically susceptible spherules referred to as miniballs are endoluminally infixed subintimally or tiny bands referred to as stays placed extravascularly by means described and illustrated in U.S. Pat. No. 11,389,171. Release into the target organ can be directly into the parenchyma through a nonjacketing side-entry connector, or from the vascular bed through a ductus side-entry or diversion side-entry jacket on the blood supply.

In addition to allowing the modulation of flow past the jacket or adjustment in the force profile and velocity of blood or urine as it passes the jacket, extending the diversion chute into the lumen detains the particles for more thorough uptake into the luminal lining on the chute entry side. Ductus side entry diversion jackets offer capabilities for controlling the uptake of a magnetized dispersant from the blood or urine circulating past the jacket. If the jacket is a vascular servovalve with motorized chute, the degree and timing of extension can be timed to the moment of infusion.

If the infusate is to be kept from continued passage through the circulation, a second jacket downstream from the first is used to release a reversal agent (counteractant, neutralizer). Neodymium permanent magnet-incorporating diversion jackets can also draw superparamagnetic iron oxide nanoparticles or magnetically susceptible drug carrier bound drugs against, or if electromagnetic, into the intima. These processes, alone and in combination, subsume significant palliative, anti-infective, and curative value. Lesions along the urinary tract or the vascular tree can be treated thus. The uses of these in medicine, alone or in combination, are many, and include the direct targeting of drugs such as anti-inflammatoires, immunosuppressants, antimicrobials, drugs that present more than one of these attributes, and radiofrequency warming of the tissue into which the ferrous material has been infixed or drawn.

Also shown in application Ser. Nos. 14/121,365 and 15/998,002 are ductus side-entry jackets which incorporate a wraparound tungsten ‘heavy’ alloy radiation shield for use with low to moderate dose-rate short half-life radionuclides, and jackets which incorporate both magnetic and radiation shielding layers. These various capabilities can be used alone or in combination to attract magnetically susceptible and/or radioactive matter, against or into the intima. The use of radioactive substances requires that not just the jacket but the line the line or lines used to deliver the substance be radiation shielded. These substances can be infused or delivered into the ductus from a port at the body surface through a jacket accessory channel.

The special ability of magnetized diversion and choke jackets is that in addition to the ability to precisely affect sensor feedback reported adjustments in local blood pressure, the associated reduction in velocity on the pressurized side of the chute, dome, or cone, detains these, effectively serving as adjustable nanoparticle traps. Where a radioactive ferrofluid not completely taken up within the desired segment should be prevented from continued travel through the circulation, one or more takeoff's, or traps, are created. A trap or SPION (superparamagnetic iron oxide nanoparticle) trap consists of positioning a choke jacket, which can be a dome, cone, or chute jacket with or without mainline to allow the direct targeting of drugs to the target or visually examine the segment through a fine fiberoptic scope.

If the segment is short, the jacket is positioned just below the segment through which reduced blood flow velocity is desired to increase the blood pressure above it. As with any application of side-entry valves, control can be servomotor driven and thus continuously or intermittently variable responsive to sensor feedback to the automatic disorder response system master control microprocessor, and simpler standalone rather than hierarchical control systems can make use of a tiny biomedically encapsulated microcontroller.

If the segment.is long, a jacket is positioned just above the upstream margin of the segment and one or more additional jackets placed at intervals downstream to the first, thereby sustaining the pressure increase and detention of the drug over the segment. In an automated system, an implanted microcontroller capable of adaptive control takes the signals from sensors in the jacket or jackets to modulate the degree of choke and thus uptake of the drug or drugs along the target segment, regardless of whether these are magnetically susceptible, and actuates the subdermally positioned flat drug storage reservoir pumps to deliver medication to each jacket in a coordinated manner in accordance with the prescription-program.

Using the three means for establishing pressure change—the use of a chute choke upright or inverted, or differently shaped choke, such as a dome or cone, and the degree of extension into the lumen, the pressure can be dynamically modulated with relative precision at intervals along a longer segment, and can do so in response to feedback from sensor probes in the jackets. In such use, the jackets serve as vascular servovalves acting as end-effectors in closed negative feedback loops. In a prosthetic disorder response system, these may represent one of several subchannels or arms within an encompassing prescription-program used to treat comorbidities where the overall action comprises a master feedback loop.

Other than higher dose rate drugs can be administered orally, or by infusion through the jacket sideline or sidelines. Exposure of the upstream endothelium or urothelium to the drug or drugs is by infusion through the jacket. If the ductus lining downstream to the segment terminus should likewise be spared exposure to the drug or drugs, a second jacket or that last in a sequence of choke jackets is used to deliver a counteractant. To allow manual control, the domes and chutes are driven by push-pull control cables adjusted with knobs in the surface port.

Ductus side-entry jacket intravascular choke servovalves can be used to accomplish local differences in blood pressure that aid in targeting drug uptake. In contrast to conventional banding, placed with fixed length to allow progressively greater constriction over time to build strength or retracted to allow increased flow, choke servovalves, or servochokes, such as that shown in FIG. 14A where radiation is sot used and FIG. 14B where it is, and FIG. 14C where that shown in FIG. 14A can have a flow obstructor or choke of any conformation, and can be adjusted in the degree of extension into the lumen either manually from the body surface port.

The latter is almost always accomplished automatically by the implanted disorder response system using a microcontroller- or in comorbid disease, a microprocessor-executing a prescription-program which acts on the basis of inputs from sensor implants in a negative feedback loop to adjust the degree of constriction. Dynamically responsive to the instantaneous action of the patient, servochoke valves should prove superior to ameroid ring and cellophane banding as used in veterinary (and probably human) practice (see, for example, Traverson, M., Lussier, B., Huneault, L., and Gatineau, M. 2018. “Comparative Outcomes between Ameroid Ring Constrictor and Cellophane Banding for Treatment of Single Congenital Extrahepatic Portosystemic Shunts in 49 Dogs (1998-2012),” Veterinary Surgery (Malden, Massachusetts) 47(2): 179-187; Konstantinidis, A. O., Adamama-Moraitou, K. K., Patsikas, M. N., and Papazoglou, L. G. 2023. “Congenital Portosystemic Shunts in Dogs and Cats: Treatment, Complications, and Prognosis,” Online, MDPI [Multidisciplinary Digital Publishing Institute] Veterinary Sciences (Basel, Switzerland) 10(5): 346).

2b(2)(j)(j1). Perivascular Sphincter-Type Flow Adjusting and Stopping Valves

One object in perivascular means for constricting a ductus is the avoidance of a presence within the lumen, where complications are common. This was a guiding factor in the extraluminal means for stenting described in U.S. Pat. No. 11,013,858, entitled Nonjacketing Side-entry Connectors and Prosthetic Disorder Response Systems. Perivascular sphincteric-type, choke-valves and servovalves are miniature versions of the electromagnetically actuated artificial sphincters shown in U.S. Pat. No. 11,759,186, entitled Ductus Side-entry Jackets and Prosthetic Disorder Response Systems, FIGS. 11 and 12.

Larger embodiments, otherwise no different than those applicable to use along smaller vascular and urinary ductus, for example, can be used to:
1. Assist weakened, paralyzed, diseased, or
2. Replace native sphincters missing due to congenital maldevelopment or injury such as one or more long the digestive tract, to include the lower esophageal (cardiac), pyloric, and ileocecal and others that allow of encirclement without extensive dissection.

Unlike sphincteric devices, which are perivascular, or cinching, choke valves are endoluminal stoppers. Unencircleable, and opening to the exterior, instituting anal blockage in fecal incontinence using mechanical means requires an endoluminal, or endorectal type, device under the immediate control of the patient. Such a device, endoluminal for rectal insertion, is addressed below in general terms in section 2b(2)(j)(j3), entitled Rectal Stopper. Sphincteric devices, to include those sequential for application along the esophagus and gut, are both side-entry jackets and valves, which may explain the coverage accorded these under both subject headings.

Fecal incontinence due to obstetric anal sphincter injuries may prove intractable to repair (see, for example, Barbosa, M., Glavind-Kristensen, M., Moller Soerensen, M., and Christensen, P. 2019. “Secondary Sphincter Repair for Anal Incontinence following Obstetric Sphincter Injury: Functional Outcome and Quality of Life at 18 Years of Follow-up,” Colorectal Disease July 26; Jango, H., Langhoff-Roos, J., Rosthoj, S., and Saske, A. 2018. “Long-term Anal Incontinence after Obstetric Anal Sphincter Injury. Does Grade of Tear Matter?,” American Journal of Obstetrics and Gynecology 218(2): 232.e1-232. e10; Şimsek, A., Ateş, M., Dirican, A., and Özgör, D. 2018. “A Surgical Technique for Secondary Repair of Obstetric Anal Sphincter Injuries; Sphinctero-vagino-perineoplasty,” Turkish Journal of Obstetrics and Gynecology 15(4): 249-253).

In many cases, the different type valves addressed here can be used interchangeably. However, the location may disallow extraluminal valves. Extraluminal, or sphincter-type flow adjusting valves servo chokevalves are described in U.S. Pat. No. 11,759,186. In contrast to tissue adapted to forcible closure, as thromboembolisms and occlusive plaque make clear-vessels expand and contract, but must not be fully occluded.

As with tapping off or diverting part of the flow to another vessel, partial constriction of a vessel, however, whether continuous, intermittent, or responsive to an automatic therapeutic command, may effect a change in local blood pressure toward a therapeutic end. Partial constriction can be applied to support an increase in the rate and completeness in upstream or retrograde uptake of a drug. This is especially so when the drug would best be tightly targeted as injurious to untargeted tissue, as with chemotherapeutics and radionuclides, for example.

However, unless prostatectomied (prostatectomized), or part of the female urinary tract, and then as to require much dissection to access, the internal urethral sphincter, or lissosphincter, does not lend itself to encirclement. Instead, an endoluminal device is needed. Accordingly, the prevention of soiling due to urinary or fecal incontinence demands an intraluminal solution. For the latter, the device must provide means for dealing with constipation or an impaction. To restrain an uncontrollable efflux of liquid stool due to a severe malabsorption syndrome, protracted viral gastroenteritis, carcinoid syndrome, or chronic diarrhea-predominant irritable bowel syndrome, for example, encircling the rectum unfeasible, only an endoluminal device can be considered.

When not an end-effector in one of a plurality of control channels each directed to a comorbid condition that demands treatment be coordinated across the set of morbid entities, command is exercised by an implanted microcontroller. Bistable control, such as entirely open or entirely closed, is by means of a tiny rotary or linear solenoid or an embedded thermal expansion wire. Continuously variable control is with tiny rotary or linear stepper or servomotors. Sphincteric-type servochokes of all types compensate for an absence or loss of contractive strength in surrounding relation to a ductus, usually within the context of more encompassing pathology, to which a different arm or channel of control in a hierarchical control system is subordinated to a master microprocessor for coordinated execution.

The smaller valves addressed are intended for use along the vascular tree, secretory ducts, ureters, and gamete-transporting ductus. In the vascular tree, use may be in coordination with flow-diverting servovalves to adjust the local blood pressure. Perivascular sphincter-type flow adjusting and stopping valves are never used to completely stop flow in the vascular tree but do function thus when used to replace a native sphincter along the gastrointestinal tract, which can be encircled with little relatively cutting. Briefly, sphincteric choke valves encircle the substrate ductus and are closed by energizing a magnet that attracts a draw plate according to the applied current.

The sensor data-fed implanted microcontroller adjusting the current, hence, the field force to the valve electromagnet in accordance with the prescription-program executed by microcontroller, sphincteric choke valves can partially or completely close off the lumen. Electromagnets portative rather than tractive, a silver wire-wound magnet with increased field force and silicon-iron crystal draw plate for significantly increased magnetic susceptibility are used. Extraluminal flow-adjusting and stopping, or choke, valves can also partially or completely close off the lumen but do so by intromission of a tongue into the lumen; that is, not by constricting the substrate ductus, but rather by controllably extending a stopper plate across the lumen.

Any type constrictive, or choke valve or servovalve, extra- or intraluminal, can be provided with one or more accessory channels to allow the direct automatic release through the valve of drugs as directed by an implanted sensor-fed microcontroller based upon a prescription-program. Also described in application Ser. No. 15/998,002 FIG. 13, sphincteric-type choke-valves are shown arranged in linear or longitudinal axial sequence to simulate peristaltic movement under the timed control of an implanted microcontroller responsive to inputs from implanted sensors. Intestinal transplantation may not make a graft available within the time necessary, and once accomplished, a gut transplant remains acutely susceptible to rejection and infection. By contrast, once the technology is able to provide functional gut within the time needed, autologous tissue engineered gut, will pose no risk of immune rejection.

Tissue engineering is not yet able to recreate human gut that encompasses the essential functions of absorption, secretion, excretion, immune function, and motility. When gut is congenitally absent, paralytic (ileus), otherwise dysfunctional, destroyed, or has been resected, normal propulsive motility and secretory function can be simulated through the addition of a sequentially coordinated train of sphincteric end actuators with accessory channels to release enzymes, for example, into the prosthetic gut. The eventual attainment of full functionality in human tissue engineered intestinal tissue will supplant other expedients, to include those electromechanical. However, until then, a train of sphincteric jackets can provide motile function. Suitability for reducing if not alleviating the symptoms of Hirschsprung's disease is probable.

Motile inadequacy one continuing shortcoming of tissue engineered gut, the combination of engineered gut which provides physiological function other than normal motility and a train of actuators to provide motility would appear to overcome the problems of graft shortage and engineered tissue motile inadequacy. That such would serve only until tissue engineered gut is perfected is obvious. Peristalsis in gamete transport ductus is primarily longitudinal rather than circumferential, but a sequential formation of miniature perivascular sphincteric valves can nevertheless be used to simulate a functional peristalsis.

Aperistaltic ureters and gamete transport tubes can also be assisted thus. When the length of gut removed is extensive, no gut extension technique such as the Bianchi procedure or serial transverse enteroplasty is applied, and no transplant attempted, the consequence may be short bowel syndrome. By contrast, prosthetic sphincters are not transplanted or engineered, and need only provide motor function. Their accessory channel or channels can be used to deliver drug therapy, and the incorporation of electrostimulatory or optogenetic end-effectors into these, for example, is obvious.

The provision of motility also applies to a linear series of sphincters under the sequential timing control of an implanted microcontroller able to simulate peristalsis in an impaired or missing esophagus. Like sphincters, the esophagus does not face the more rigorous requirements of a prosthesis that would replace an extended segment of gut, where the segment-selective absorption of different nutrients, for example, demands a substrate tube that while motility impaired, still provides absorptive, secretory, immunocytic, and excretory function.

Where such function is incomplete or lacking, oral enzymes and probiotics, for example, can compensate for the shortcomings, the patient periodically examined for side effects, such as fibrosing colonopathy. Only a length of missing gut too large for spontaneous adaptation through enlargement of the villi, increase in the diameter of the gut, and compensatory reduction in the rate of propulsion warrant consideration of replacement. Currently, allografts, while more functional than tissue engineered gut, often prove unavailable or disappoint due to infection and/or rejection.

Motility imparted by electromechanical means, the instatement or reinstatement of other function, primarily absorption, depends upon the functionality of the substrate tube, whether:
1. Transplanted as intact gut or stem cells (see, for example, Yaguchi, J. and Yaguchi, S. 2019. “Evolution of Nitric Oxide Regulation of Gut Function,” Online, Proceedings of the National Academy of Sciences of the United States of America 116(12): 5607-5612; Clouse, J. W., Kubal, C. A., Fridell, J. A., and Mangus, R. S. 2018. “Posttransplant Complications in Adult Recipients of Intestine Grafts without Bowel Decontamination,” Journal of Surgical Research 225:125-130; Hakim, B., Myers, D. T., Williams, T. R., Nagai, S., and Bonnett, J. 2018. “Intestinal Transplants: Review of Normal Imaging Appearance and Complications,” British Journal of Radiology 91(1090): 20180173; Liu, W., Zhang, L., and Wu, R. 2018. “Enteric Neural Stem Cells Expressing Insulin-like Growth Factor 1: A Novel Cellular Therapy for Hirschsprung's Disease in Mouse Model,” DNA and Cell Biology 2018 37(7): 642-648; Cooper, J. E., Natarajan, D., McCann, C. J., Choudhury, S., Godwin, H., Burns, A. J., and Thapar, N. 2017. “In Vivo Transplantation of Fetal Human Gut-derived Enteric Neural Crest Cells,” Neurogastroenterology and Motility 29(1); McCann, C. J., Cooper, J. E., Natarajan, D., Jevans, B., Burnett, L. E., Burns, A. J., and Thapar, N. 2017. “Transplantation of Enteric Nervous System Stem Cells Rescues Nitric Oxide Synthase Deficient Mouse Colon,” Nature Communications 8:15937) or
   SEE pat3, docx
2. Tissue engineered (see, for example, Barber, K., Studer, L., and Fattahi, F. 2019. “Derivation of Enteric Neuron Lineages from Human Pluripotent Stem Cells,” Nature Protocols 14(4): 1261-1279; Costa, J. and Ahluwalia, A. 2019. “Advances and Current Challenges in Intestinal in Vitro Model Engineering: A Digest,” Online, Frontiers in Bioengineering and Biotechnology 7:144; Simkin, J. E., Zhang, D., Stamp, L. A., and Newgreen, D. F. 2019. “Fine Scale Differences within the Vagal Neural Crest for Enteric Nervous System Formation,” Developmental Biology 446(1): 22-33; Srinivasan, A. and Toh, Y. C. 2019. “Human Pluripotent Stem Cell-derived Neural Crest Cells for Tissue Regeneration and Disease Modeling,” Online, Frontiers in Molecular Neuroscience 12:39; Zhang, D., Rollo, B. N., Nagy, N., Stamp, L., and Newgreen, D. F. 2019. “The Enteric Neural Crest Progressively Loses Capacity to Form Enteric Nervous System,” Developmental Biology 446(1): 34-42; Zhou, W., Chen, Y., Roh, T., Lin, Y., Ling, S., and 7 others 2018. “Multifunctional Bioreactor System for Human Intestine Tissues,” American Chemical Society Biomaterials Science and Engineering 4(1): 231-239; Chen, Y., Zhou, W., Roh, T., Estes, M. K., and Kaplan, D. L. 2017. “In Vitro Enteroid-derived Three-dimensional Tissue Model of Human Small Intestinal Epithelium with Innate Immune Responses,” Online, PLoS [Public Library of Science] One 12(11): e0187880; Schlieve, C. R., Fowler, K. L., Thornton, M., Huang, S., Hajjali, 1., and 4 others 2017. “Neural Crest Cell Implantation Restores Enteric Nervous System Function and Alters the Gastrointestinal Transcriptome in Human Tissue-Engineered Small Intestine,” Stem Cell Reports 9(3): 883-896; Trecartin, A. and Grikscheit, T. 2017. “Tissue Engineering Functional Gastrointestinal Regions: The Importance of Stem and Progenitor Cells,” Cold Spring Harbor Perspectives in Medicine 7(10). pii: a025700; Workman, M. J., Mahe, M. M., Trisno, S., Poling, H. M., Watson, C. L., and 13 others 2017. “Engineered Human Pluripotent-stem-cell-derived Intestinal Tissues with a Functional Enteric Nervous System,” Nature Medicine 23(1): 49-59; Bitar, K. N., Raghavan, S., and Zakhem, E. 2014. “Tissue Engineering in the Gut: Developments in Neuromusculature,” Gastroenterology 146(7): 1614-1624; Watson, C. L., Mahe, M. M., Munera, J., Howell, J. C., Sundaram, N., and 11 others 2014. “An in Vivo Model of Human Small Intestine Using Pluripotent Stem Cells,” Nature Medicine 20(11): 1310-1314; Bitar, K. N. and Raghavan, S. 2012. “Intestinal Tissue Engineering: Current Concepts and Future Vision of Regenerative Medicine in the Gut,” Neurogastroenterology and Motility 24(1): 7-19).

Unlike stopper type choke valves (choke or check) servovalves, which adjust the cross-sectional area for flow-through from within the lumen, sphincteric or perivascular (girth, belt, sash) type servo chokevalves about the substrate ductus in order to compress it in diameter from the about its outer surface. Application thus is adequate in most gastrointestinal, urogenital, and vascular ductus; however, not all ductus can be grappled about, and injury or disease of the tissue at the surface may necessitate an intraluminal device.

2b(2)(j)(j2). Intraluminal Flow-Adjusting Servo Chokevalves, or Servochokes

Shown in FIG. 14A is a lumen-intromissive rate flow-adjusting servo chokevalve, or servochoke, a servomotor-driven, continuously variable luminal obturator device to adjust the pressure and velocity of blood flow without the need for a sidestem outlet, or fluid takeoff (mainline or bloodline). As do inline coupling jackets, the absence of a protrusive sidestem liberalizes its positionability for use with great to medium size vessels anywhere in circulatory system. Where inline coupling jackets serve to secure a vascular prosthesis to the stump at either end, a servochoke is used to adjust the blood pressure without the need to tap off a volume of blood for diversion to another vessel, and where a shunt can only continuously variably lower the pressure, a servochoke can both increase or lower it.

True to the Bernoulli Effect, for a given pump outlet pressure and velocity, constricting the cross-sectional area of a hose connected to a pump to a measured extent increases the pre-obstruction (pre-servochoke, upstream) pressure and lowers its velocity, while the effect on the post-obstruction (downstream, post-servochoke) pressure is the opposite, that is, the pressure has been lowered and the velocity increased in proportion to the degree of obstruction (see, for example, Brown, M. L, Liffman, K., Semmens, J. and Sutalo, I. 2011. “Vascular Arterial Haemodynamics,” in Fitridge, R. and Thompson, M. Chapter 8. Mechanisms of Vascular Disease: A Reference Book for Vascular Specialists, Adelaide, Australia: Barr Smith Adelaide University Press, pages 153-176).

In vascular terms, such a device allows adjusting the pressure and velocity of blood flow in a controllable manner to increase or reduce the blood pressure without the need to tap off a portion of the flow for diversion into another larger vessel. Local blood pressure reduction without the need for a protrusive sidestem and shunt expedites and allows placement in tight locations where a shunt would encroach upon neighboring tissue.

Another type of continuously variable servochoke which effects much the same action as an obturator type by acting as a sphincter to reduce or increase the luminal cross-section is described above in section 2b(2)(j)(j1), entitled Perivascular Sphincter-type Flow Adjusting and Stopping Valves, one such shown in FIGS. 11 and 12 of. S. U.S. Pat. No. 11,759,186, entitled Ductus Side-entry Jackets and Prosthetic Disorder Response Systems.

Servochokes have two major applications—to allow a less intense magnetic field, hence, a less powerful magnet, extract a magnetically susceptible carrier-bound drug, metabolite, or analyte from blood with its pressure lowered, and to temporarily reduce the perfusion, hence, lower the oxygen saturation, of the malignancy-unaffected tissue in an organ or volume of tissue to reduce its radiosensitivity while delivering oxygenating, hence radiosensitizing agents into the carcinoma or carcinosarcoma (sarcocarcinoma) through a side-entry valve jacket or vascular servovalve feeding a radioactive drip (see, for example, Wei, S. T., Chiang, J. Y., Wang, H. L, Lei, F. J., Huang, Y. C., and 3 others 2023. “Hypoxia-induced CXC [two N-terminal cysteines of CXC chemokines separated by one amino acid] Chemokine Ligand 14 Expression Drives Protumorigenic [pro tumorigenic] Effects through Activation of Insulin-like Growth Factor-1 Receptor Signaling in Glioblastoma,” Online, Cancer Science (Oxford, England) 114(1): 174-186; El-Benhawy, S. A., Sakr, O. A., Fahmy, E. I., Ali, R. A., Hussein, M. S., and 4 others 2022. “Assessment of Serum Hypoxia Biomarkers Pre- and Post-radiotherapy in Patients with Brain Tumors,” Online, Journal of Molecular Neuroscience (Totowa, New Jersey) 72(11): 2303-2312; Horsman, M. R., Sorensen, B. S., Busk, M., and Siemann, D. W. 2021. “Therapeutic Modification of Hypoxia,” Clinical Oncology (Royal College of Radiologists (Great Britain)(London, England) 33(11): e492-e509; Graham, K. and Unger, E. 2018. “Overcoming Tumor Hypoxia as a Barrier to Radiotherapy, Chemotherapy, and Immunotherapy in Cancer Treatment,” Online, International Journal of Nanomedicine (Auckland, New Zealand) 13:6049-6058; Jahanban-Esfahlan, R., de la Guardia, M., Ahmadi, D., and Yousefi, B. 2018. “Modulating Tumor Hypoxia by Nanomedicine for Effective Cancer Therapy,” Journal of Cellular Physiology (New York, New York) 233(3): 2019-2031; Horsman, M. R. and Overgaard, J. 2016. “The Impact of Hypoxia and Its Modification of the Outcome of Radiotherapy,” Online, Journal of Radiation Research (Oxford, England) 57 Supplement 1:190-198; Rockwell, S., Dobrucki, I. T., Kim, E. Y., Marrison, S. T., and Vu, V. T. 2009. “Hypoxia and Radiation Therapy: Past History, Ongoing Research, and Future Promise,” Online, Current Molecular Medicine (Boca Raton, Florida) 9(4): 442-458; Hoogsteen, I. J., Marres, H. A., van der Kogel, A. J., and Kaanders, J. H. 2007. “The Hypoxic Tumour Microenvironment, Patient Selection and Hypoxia-modifying Treatments,” Clinical Oncology (Royal College of Radiologists (Great Britain) (London, England) 19(6): 385-396; Weinmann, M., Welz, S., and Bamberg, M. 2003. “Hypoxic Radiosensitizers and Hypoxic Cytotoxins in Radiation Oncology,” Online, Current Medical Chemistry Anticancer Agents (San Francisco, California) 3(5): 364-374).

In FIG. 14C, part number 100 is a radiation-shielded body surface port backplate, 102 a shielded drug reservoir, 103 a shielded drug outlet pump or drip stopcock, 8 and 8′ shielded druglines, 165 a shielded nonjacketing side-entry connector, and 166 a vascular servochoke as shown longitudinally sectioned in FIG. 14A. The servochoke lowers the oxygen level of the cancer-unaffected tissue surrounding the tumor to reduce its radiosensitivity while delivering oxygenating, hence radiosensitizing agents into the carcinoma or carcinosarcoma (sarcocarcinoma) by alternating between two extents of its intraluminal obturator intromission into the lumen in synchrony with the application of radiation.

The range of alteration at the extremes is zero and 100 percent, with practical settings more conservative. Thus, the reduction in oxygenation of the cancer-free tissue is accomplished mechanically, by literally interrupting its blood supply at intervals too brief to cause harm. Lacking a protrusive sidestem, a servochoke is more expeditiously positioned and in locations that a sidestem would prohibit. This can also serve to increase the upstream pressure to intentionally dilate the vessel, increase the rate of take-off upstream at an adjustable rate and pressure, and/or increase the uptake of medication intended to be absorbed into the endothelium or urothelium.

The same as vascular servovalves in terms of motor control, an intraluminal servo chokevalve, or servochoke, incorporates an obturator, or flat stopper plate, 43 in FIG. 14A rather than a curved flow diversion chute, or tongue, part number 18 in a vascular valve or servovalve which allows a portion of the flow through the substrate ductus to be diverted, or redirected, into another ductus, such as required in switch transplantation. Under the control of an implanted microcontroller responsive to implanted sensors, the valve extends or intromits its stopper plate 43 into the substrate lumen a luminal flow-through cross-section reducing plate, or tongue.

Such contemplates that an intraluminal servo chokevalve of this kind can be applied to the blood supply of the gut, along the celiac axis, superior, and/or the inferior mesenteric arteries—to reduce the local blood pressure in proportion to the degree of increase velocity of flow. Used in moderation this may reduce hypermotility in different disease states. Another device intended to prevent soiling due to projectile fecal incontinence such as caused by cryptosporidiosis, for example, is a “dumb” rectal channel stopper addressed in the section that follows, 2b(2)(j)(j3), entitled Rectal Stopper.

As addressed in U.S. Pat. No. 11,389,171, entitled Integrated System for the Infixion and Retrieval of Implants, the potential application of basic side-entry jackets to automatically release medication at lesions along the bowel has palliative if not curative potential (see, for example, Gajendran, M., Loganathan, P., Catinella, A. P., and Hashash, J. G. 2018. “A Comprehensive Review and Update on Crohn's Disease,” Disease-A-Month 64(2): 20-57; Grubelic Ravić, K., Paić, F., Vucelić, B., Brinar, M., Čuković-Čavka, S., and 5 others 2018. “Association of Polymorphic Variants in Serotonin Re-uptake Transporter Gene with Crohn's Disease: A Retrospective Case-control Study,” Croatian Medical Journal 59(5): 232-243; Burisch, J. 2014. “Crohn's Disease and Ulcerative Colitis. Occurrence, Course and Prognosis during the First Year of Disease in a European Population-based Inception Cohort,” Danish Medical Journal 61(1): B4778).

Essentially limited to application along the arterial tree rather than the gut or secretory ducts then, the degree of extension into the lumen determines the resistance to flow and pressure buildup which can be used to adjust the local blood pressure. Increasing the extension into the lumen proportionally increases the back-pressure, or pressure upstream to the tongue and reduces the pressure while increasing the velocity of the outflow. This reduction in pressure then can be used to increase the uptake of a magnetically susceptible carried drug or drugs released through an accessory channel (sideline, drugline) in the valve.

Such capability is useful for targeting and preventing higher dose rate radionuclides, potentially harmful chemotherapeutics, and potent steroids, for example, from continued flow. If necessary, uptake can be further enforced by binding the drug or drugs to superparamagnetic carriers. That any such valves can incorporate a magnetized layer is focused upon in U.S. Pat. No. 11,759,186, entitled Ductus Side-entry Jackets and Prosthetic Disorder Response Systems.

2b(2)(j)(j3). Rectal Stopper

In addition to infection of the distal small intestine, fecal incontinence can be caused by hemorrhoids, fecal overflow, pelvic floor weakness, rectum emptying disorders, loss of rectal storage capacity, pregnancy, after pregnancy for up to about three months, rectal prolapse, rectocele, and physical inactivity. An improved fecal incontinence plug (rectal stopper, anal stopper, anal plug, anal insert) which is disposed of by dropping directly into the toilet immediately before voiding is fusiform (spindle) or allantoidal (torpedo) in shape when inserted (see, for example, Shah, R. and Villanueva Herrero. J. A. 2022. “Fecal Incontinence,” Online, Treasure Island, Florida: StatPearls Publishing Co.).

Bowel evacuation disorders include motility-related diarrheic irritable bowel syndrome associated with severe pain, for example, where the use of opioids to reduce the pain slow peristalsis causing constipation as well as the hypermotility-induced diarrhea would best be much reduced if not eliminated. Acutely distressing is the sudden and unanticipated loss of diarrheic stool into an undergarment while urinating in a public urinal, a circumstance which a rectal stopper would prevent.

As with any implanted system electromechanical end effector device such as the ‘tuned’ shunt shown in FIG. 22F or an electrostimulatory neuromodulator, for example, the device itself is directed to the immediate and local mechanical counteraction of a dysfunction such as portal or pulmonary hypertension, or as here addressed, chronic diarrhea, which symptomatic of an underlying causative agent such as an excess of magnesium in the diet or disease, does not directly target the causative factor and does not constitute a cure. However, as explained above in section 2b(2)(h)(h1), entitled The ‘tuned’ shunt, such an electromechanical end-effector is not limited to sensor feedback controlled by a suitably encapsulated but otherwise relatively simple implanted microcontroller chip.

Here where the device addressed is manually rather than electrically operated without a direct connection to a control system, the opportunity to emphasize the potential of a prosthetic disorder response system as described in U.S. Pat. No. 11,759,186, entitled Ductus Side-entry Jackets and Prosthetic Disorder Response Systems and in copending application Ser. No. 17/689,880, entitled Prosthetic Disorder Response Systems to support such a device by counteracting the causative factor or factors to effectively approximate if not secure a cure is even more pronounced than when the device is electromechanical and controlled by the automated system.

While the manually operated device is related to the wider disorder response system only in that sensor in it signals the controller as to its use, the addition of the control system can minimize if not eliminate further need for its use. More specifically, with the addition of sensors appropriately positioned in the digestive tissues and organs to monitor peristalsis and the secretion of gastric acid, digestive enzymes, and hormones, and if necessary, electrostimulatory neuromodulators to affect the rate of movement, a control microprocessor can control the release of malabsorption-remediating drugs and agents from small drug reservoirs implanted subcutaneously in the pectoral region.

The directly pipe-targeted delivery of these means that their concentration, hence, each dose, can be greater than if directly introduced into the circulatory system, this factor allowing the use of small reservoirs. Digestive function is complex to the extent that to monitor and treat it, a master control microprocessor and not just a simple microcontroller chip is essential to support it.

### Gastroesophageal Reflux Disease

Gastroesophageal reflux and constipation are related. Gastroesophageal reflux in relation to bronchiolitis obliterans in lung transplantation is addressed above in sections 2b, entitled Vascular Valves and Servovalves in the Diversion of Blood Flow, and 2b(1)(b)(b9), entitled Single Living and Single or Double Deceased Donor Metered Compound Vascular Bypass, or Switched-organ, Lung Transplantation. In the very young, the very old, and the unmanageable, a simple automatic disorder response system consisting of a suitably encapsulated microcontroller chip, drug reservoir, drugline, hyperacidity sensor, and side-entry jacket anywhere along the arterial tree can be implanted to detect the need for and automatically release a hyperacidity counteractant such as esomeprazole or famotidine.

A relation between gastroesophageal reflux and constipation is well documented (see, for example, Al Jalal, B. A., Alnawah, A. K., Alturkie, F. M., Alqadhib, K. J., Alabbadi, M. S., Ali, S. I., and Al Hussaini, H. A. 2024. “Overlap between Gastroesophageal Reflux Disease and Functional Constipation and Associated Risk Factors among the General Population in Saudi Arabia,” International Journal of General Medicine (Auckland, New Zealand) 17:669-683; Ogasawara, N., Kasugai, K., Funaki, Y., Ebi. M., Izawa, S., and 6 others 2023. “Relationships between Body Mass Index and Constipation, Gastroesophageal Reflux Disease, Stool Forms Based on the Bristol Stool Form Scale, and Education Level: Results from an Internet Survey in Japan,” Online, Journal of Clinical Biochemistry and Nutrition (Mitake, Gifu, Japan) 73(1): 84-90; Ahmadipour, S., Salami-Khaneshan, A., Farahmand, F., and Baharvand, P. 2022. “Co-occurrence of Childhood Functional Constipation and Gastroesophageal Reflux Disease (GERD),” Online, Annals of Medicine and Surgery (London, England) 74:103302; Ogasawara, N., Funaki, Y., Kasugai, K. Ebi, M., Tamura, Y., Izawa. S., and Sasaki, M. 2022. “Overlap between Constipation and Gastroesophageal Reflux Disease in Japan: Results from an Internet Survey, Online, Journal of Neurogastroenterology and Motility (Seoul, South Korea) 28(2): 291-302); Dehghani, S. M., Poorghaiomi. R., and Javaherizadeh, H. 2020. “Clinical Manifestation of Gastroesophageal Reflux among Children with Chronic Constipation,” Online, Middle East Journal of Digestive Diseases (Tehran, Iran) 12(3): 178-181; Baran, M., Cagan Appak, Y., Karakoyun, M., Yalcinkaya, S., Eliacik, K., and Dundar, B. N. 2017. “The Overlap of Gastroesophageal Reflux Disease and Functional Constipation in Children: The Efficacy of Constipation Treatment,” European Journal of Gastroenterology and Hepatology (London, England) 29(11): 1264-1268).

Some disorders such as intractable gastroesophageal reflux disease may necessitate measures beyond the capability of the implanted system, such as a hiatal herniorrhaphy or laparoscopic fundoplication, (see, for example, Bonavina, L., Horbach, T., Schoppmann, S. F., and DeMarchi, J. 2021. “Three-year Clinical Experience with Magnetic Sphincter Augmentation and Laparoscopic Fundoplication” Online, Surgical Endoscopy ((New York, New York)) 35(7): 3449-3458; Mckinley, S. K., Dirks. R. C., Walsh, D., Hollands. C., Arthur, L. E., and 6 others 2021. “Surgical Treatment of GERD: Systematic Review and Meta-analysis,” Surgical Endoscopy (New York, New York) 35(8): 4095-4123; Stanak, M., Erdos, J., Hawlik, K., and Birsan, T. 2018. “Novel Surgical Treatments for Gastroesophageal Reflux Disease: Systematic Review of Magnetic Sphincter Augmentation and Electric Stimulation Therapy,” Online, Gastroenterology Research (Québec. Canada) 11(3): 161-173).

However, the system can manage most other disorders related to malabsorption to include those involving an inborn error of metabolism, through sensor-triggered medicinal responses, and where appropriate, electrostimulation. Proper digestion essential for normal growth and development, such encompasses the administration of insulin, for example, and the automatic direct pipe-targeting to the appropriate site of virtually any hormone, enzyme, or drug used to treat a disorder of absorption, metabolism, and/or the gastrointestinal tract.

Seen thus, the relevance of such treatment extends to almost every disorder addressed by internal medicine, to include numerous conditions that result in intrauterine or fetal growth restriction to prompt the placement of appropriate sensors in the gravida, umbilical cord, and fetus as addressed above in section 2a, entitled Background of the Invention. General, some conditions potentially remediable using gene therapy likewise addressed in section 2a above, and errors in metabolism that impair normal development as well as other disorders (see, for example, (Adam, M. P., Feldman, J., Mirzaa, G. M., Pagon, R. A., Wallace, S. E., and 4 others (eds.) 2024. GeneReviews. Seattle, Washington: University of Washington Press; Chen, S., Heendeniya, S. N., Le, B. T., Rahimizadeh, K., Rabiee. N., Zahra, Q. U. A., and Veedu, R. N. 2024. “Splice-modulating Antisense Oligonucleotides as Therapeutics for Inherited Metabolic Diseases,” Online, BioDrugs: Clinical Immunotherapeutics, Biopharmaceuticals and Gene Therapy (Auckland, New Zealand) 38(2): 177-203; Redondo-Florez, L., Rubio-Zarapuz, A., Martin-Rodriguez, A., and Tornero-Aguilera, J. F. 2024. “Microbiota Implications in Endocrine-related Diseases: From Development to Novel Therapeutic Approaches, Online, MDPI [Multidisciplinary Digital Publishing Institute] Biomedicines (Basel, Switzerland). 12(1): 221; Cossu, M., Pintus, R., Zaffanello, M., Mussap. M., Serra, F., Marcialis, M. A., and Fanos, V. 2023. “Metabolomic Studies in Inborn Errors of Metabolism: Last Years and Future Perspectives,” Online, MDPI [Multidisciplinary Digital Publishing Institute] Metabolites (Basel, Switzerland) 13(3): 447; van de Burgt, N., van Doesum W. Grevink M, van Niele S. de Koning T. and 4 others 2023. “Psychiatric Manifestations of Inborn Errors of Metabolism: A Systematic Review,” Online, Neuroscience and Biobehavioral Reviews 144:104970; Isenmann, M., Stoddart, M. J., Schmelzeisen, R., Gross. C., Della Bella, E., and Rothweiler, R. M. 2023. “Basic Principles of RNA Interference: Nucleic Acid Types and in Vitro Intracellular Delivery Methods,” Online, MDPI [Multidisciplinary Digital Publishing Institute] Micromachines (Basel, Switzerland) 14(7): 1321; Jakubek-Kipa. K., Galiniak, S., Lagowska, K., Krupa, I., Ludwin, A., Tabarkiewicz, J., and Mazur. A. 2023. “Bioleptin as a Useful Marker of Metabolic Status in Children with Diabetes Mellitus Type 1,” Online, Frontiers in Endocrinology (Lausanne, Switzerland) 14:1235409; Jeanmonod, R., Asuka, E., and Jeanmonod, D. 2023. “Inborn Errors of Metabolism,” Online, Treasure Island, Florida: StatPearls Publishing Co.; Liu, L., Zhang, L., Li, C., Qiu, Z., Kuang. T., Wu Z., and Deng. W. 2023. “Effects of Hormones on Intestinal Stem Cells,” [BioMed Central] Stem Cell Research and Therapy (London, England) 14(1): 105; Senarathne, U. D., Indika, N. R., Jezela-Stanek, A., Ciara, E., Frye. R. E., Chen. C., and Stepien, K. M. 2023. “Biochemical, Genetic, and Clinical Diagnostic Approaches to Autism-associated Inherited Metabolic Disorders,” Online, MDPI [Multidisciplinary Digital Publishing Institute] Genes (Basel, Switzerland) 14(4): 803; Tafuri, S. M. and Lui, F. 2023. “Embryology, Anencephaly,” Online, Treasure Island, Florida: StatPearls Publishing Co.; Zhou, P., Wang, X., Sun, M., and Yan S. 2023. “Effects of Natural Products on Functional Constipation: Analysis of Active Ingredient and Mechanism,” Naunyn-Schmiedeberg's Archives of Pharmacology October 2023; Zuvarox, T. and Belletieri. C. 2023. “Malabsorption Syndromes,” Online, Treasure Island, Florida: StatPearls Publishing Co; Barresi, V., Musmeci, C., Rinaldi, A., and Condorelli, D. F. 2022. “Transcript-targeted Therapy Based on RNA Interference and Antisense Oligonucleotides: Current Applications and Novel Molecular Targets,” Online, MDPI [Multidisciplinary Digital Publishing Institute] International Journal of Molecular Sciences (Basel, Switzerland) 23(16): 8875; Liang, Z. and Yun, C. C. 2022. “Compensatory Upregulation of LPA: [lysophosphatidic acid] and Activation of the PI3K [phosphoinositide, or phosphatidylinositol, 3-kinase]-Akt [protein kinase B] Pathway Prevent LPA [lysophosphatidic acid]s-dependent Loss of Intestinal Epithelial Cells in Intestinal Organoids,” Online, MDPI [Multidisciplinary Digital Publishing Institute] Cells (Basel, Switzerland) 11(14): 2243; Mori, H., Verbeure, W., Schol, J., Carbone, F., and Tack, J. 2022. “Gastrointestinal Hormones and Regulation of Gastric Emptying,” Current Opinion in Endocrinology, Diabetes. and Obesity (London, England) 29(2): 191-199; Marquez-Caraveo. M. E., Ibarra-Gonzalez, I., Rodriguez-Valentin, R., Ramirez-Garcia, M. A., Perez-Barron, V., Lazcano-Ponce, E., and Vela-Amieva, M. 2021. “Brief Report: Delayed Diagnosis of Treatable Inborn Errors of Metabolism in Children with Autism and Other Neurodevelopmental Disorders, Journal of Autism and Developmental Disorders (New York, New York) 51(6): 2124-2131; Zigman, T., Petković Ramadza, D., Simic, G., and Barić, I. 2021. “Inborn Errors of Metabolism Associated with Autism Spectrum Disorders: Approaches to Intervention,” Online, Frontiers in Neuroscience (Lausanne, Switzerland) 15:673600; Sun, L. J., Li, J. N., and Nie, Y. Z. 2020. “Gut Hormones in Microbiota-Gut-Brain Cross-talk,” Online, Chinese Medical Journal (English edition of Zhonghua vi Xue Za Zhi)(Beijing, China) 133(7): 826-833; Ahmed, M. and Ahmed, S. 2019. “Functional, Diagnostic, and Therapeutic Aspects of Gastrointestinal Hormones,” Online, Gastroenterology Research (Québec, Canada) 12(5): 233-244; Camilleri, M. 2019. “Gastrointestinal Hormones and Regulation of Gastric Emptying, Online, Current Opinion in Endocrinology, Diabetes, and Obesity (London, England) 26(1): 3-10; Dubrovsky, G., Lo, Y. K., Wang, P. M., Wu, M. D., Huynh, N., Liu, W., and Dunn J. C. Y. 2019. “Intestinal Electrical Stimulation to Increase the Rate of Peristalsis,” Journal of Surgical Research (New York, New York) 236:153-158; de Camp, N. V., Heimann, A., Kempski, O., and Bergeler J. 2018. “Accelerometer-based Assessment of Intestinal Peristalsis: Toward Miniaturized Low-power Solutions for Intestinal Implants,” Online IEEE [Institute of Electrical and Electronics Engineers] Journal of Translational Engineering in Health and Medicine 6:2700507; Li, D., Mastaglia, F. L., Fletcher, S., and Wilton, S. D. 2018. “Precision Medicine through Antisense Oligonucleotide-mediated Exon Skipping,” Trends in Pharmacological Sciences (Barking, East London, England) 39(11): 982-994; Saudubray, J. M. and Garcia-Cazorla, A. 2018. “An Overview of Inborn Errors of Metabolism Affecting the Brain: From Neurodevelopment to Neurodegenerative Disorders,” Online, Dialogues in Clinical Neuroscience (Abingdon-on-Thames, Oxfordshire, England) 20(4): 301-325; Pfeiffer, R. F. 2014. “Neurologic Manifestations of Malabsorption Syndromes,” Handbook of Clinical Neurology 120:621-632; Thomas, R. P., Hellmich, M. R., Townsend, C. M. Jr., and Evers, B. M. 2003. “Role of Gastrointestinal Hormones in the Proliferation of Normal and Neoplastic Tissues,” Endocrine Reviews (New York, New York) 24(5): 571-599).

Hypomotility and impaction induced by an opioid prescribed to alleviate pain is familiar in the emergency room. Hypermotility and diarrhea alone are usually painless and can be reduced with the aid of an antidiarrheal, loperamide and bismuth subsalicylate, those most common sold over the counter; however, an antidiarrheal can itself cause pain, and the pain in irritable bowel syndrome can persist even if the hypermotility is controlled. With the reduction in inflow less than causes pain, the object is to reduce if not eliminate both the hypermotility and the pain, and therewith, reduce if not eliminate the need for medication.

Based upon the severe symptoms of intestinal pseudo-obstruction, often associated with systemic lupus erythematosus, for example, the problem arises secondary to opioid-induced primary motile interference that slows peristalsis and can cause constipation (see, for example, Barros, L. L., Farias, A. Q., and Rezaie, A. 2019. “Gastrointestinal Motility and Absorptive Disorders in Patients with Inflammatory Bowel Diseases: Prevalence, Diagnosis and Treatment,” World Journal of Gastroenterology 25(31): 4414-4426; Kellow, J. E., Phillips, S. F., Miller, L. J., and Zinsmeister, A. R. 1988. “Dysmotility of the Small Intestine in Irritable Bowel Syndrome,” Gut 29(9): 1236-1243) is contraindicated (see, for example, Adler, B. L., Timlin, H., and Birnbaum, J. 2019. “Lupus Intestinal Pseudo-obstruction and Hydronephrosis: Case Report,” Medicine (Baltimore, Maryland) 98(28): e16178; Pironi, L. and Sasdelli, A. S. 2019. “Management of the Patient with Chronic Intestinal Pseudo-obstruction and Intestinal Failure,” Gastroenterology Clinics of North America 48(4): 513-524; Downes, T. J.1, Cheruvu, M. S., Karunaratne, T. B., De Giorgio, R., and Farmer, A. D. 2018. “Pathophysiology, Diagnosis, and Management of Chronic Intestinal Pseudo-obstruction,” Journal of Clinical Gastroenterology 52(6): 477-489; El-Chammas, K. and Sood, M. R. 2018. “Chronic Intestinal Pseudo-obstruction,” Clinics in Colon and Rectal Surgery 31(2): 99-107; Magen, E. and Chikovani, T. 2016. “Possible Therapeutic Role of IgE Blockade in Irritable Bowel Syndrome,” World Journal of Gastroenterology 22(43): 9451-9456; Xu, N., Zhao, J., Liu, J., Wu, D., Zhao, L., and 12 others 2015. “Clinical Analysis of 61 Systemic Lupus Erythematosus Patients with Intestinal Pseudo-obstruction and/or Ureterohydronephrosis: A Retrospective Observational Study,” Medicine (Baltimore, Maryland) 94(4): e419). The placement of basic side-entry jackets to target histamine receptors in irritable bowel syndrome, however, is not contraindicated (see, for example, Fabisiak, A., Włodarczyk, J., Fabisiak, N., Storr, M., and Fichna, J. 2017. “Targeting Histamine Receptors in Irritable Bowel Syndrome: A Critical Appraisal,” Journal of Neurogastroenterology and Motility (Seoul, South Korea) 23(3): 341-348).

An improved fecal incontinence plug (rectal stopper, anal stopper, anal plug, anal insert) which is disposed of by dropping directly into the toilet immediately before voiding is fusiform (spindle) or allantoidal (torpedo) in shape when inserted.

The insert is inflated by squeezing a dependent rubbery bulb which admits air through a one-way valve and deflates through a slit which opens when the bulb is pulled to remove the device. The incontinence plug consists of a body of elastic material with a frame of pliant longitudinal ribs, or spines, running the length of the device from one end to the other to maintain its shape during insertion. Neither solid nor fixed in shape, there is no discomfort due to protrusion along the anal canal or migration, no difficult insertion, removal, or retention even though sizing or fitting to the individual patient is unnecessary.

Placement in the anal canal of a temporary device to prevent involuntary soiling and contamination associated with diarrheic disease, especially when contagious as gastroenteritis allows a patient with uncontrollable diarrheic incontinence the freedom to pursue daily activities without interruption or the risk of spreading infection to others. Pending resolution, an uncontrollable loss of stool prevents participation in normal activities, limits the patient to nearby facilities, and poses a health threat to others, especially those with weakened immune systems, and in the clinic, making a device that provides control significant.

Diarrheic conditions include those infectious, or gastroenteritic, such as cryptosporidiosis, cyclosporiasis, Clostridium difficile, cholera, and dysentery, and those neuroendocrine, such as carcinoid syndrome; functional gastrointestinal disorder, such as irritable bowel syndrome; and inflammatory bowel disease, such as Crohn's disease and ulcerative colitis. Profuse watery stool is often a debilitating effect with such infections. Of these, an insertable rectal stopper is intended for temporary or episodic use.

For infectious diarrhea, the device will most often be needed for no more than two weeks and can be made as disposable. Quick removal is by deflation by squeezing the bulb which extends out through the anus. Resilient axially longitudinal pines running along the length of the device maintain its shape during insertion. Squeezing the bulb inflates, while pulling on it deflates, the device. After defecation and removal, the device is discarded. A different embodiment devised to avoid the need to carry several where voiding is frequent can allow the device to be washed, disinfected, and reinserted.

The device contains a biocompatible, nonirritating antiseptic within the viscid gel, and once inserted, squeezing the dependent bulb causes it to expand such as by means of a one-way air valve, with some of the antiseptic oozing out of small pores about the surface of the device as it expands. The medicated gel is insoluble in water. Accordingly, successive uses will eventually deplete the antiseptic as to reduce the snugness of fit within the anal canal, whereupon whether designed for single or multiple use, the device is discarded. In cryptosporidiosis, for example, where the stool consists almost entirely of water which the native internal and external sphincters are unable to stop from passing, the expulsion of stool is without an aural, or preliminary warning sensation, sudden, large in volume, projectile, and completely uncontrollable, preventing the pursuit of normal activities.

Such an anal stopper avoids medication that is mostly inadequate to control the problem and carries risks. The use of an over-the-counter antidiarrheal, typically loperamide hydrochloride (trade name Imodium) or bismuth subsalicylate (trade name Kaopectate) scarcely affects a watery infectious diarrhea such as that caused by cryptosporidiosis and some cases of dysentery. Moreover, the use of loperamide and other opioids to slow intestinal motility are never likely to dispel a watery diarrhea to any significant extent, but are likely to provoke adverse side effects. Loperamide is an opioid that risks nausea, sedation, and inadequate ventilation, while the excessive use of bismuth subsalicylate risks salicylism with neurotoxicity, gastritis, constipation, and encephalopathy.

Whereas, sphincteric-type choke servovalves, or servochokes such as that shown in FIG. 14 and in FIGS. 11 and 12 of U.S. Pat. No. 11,759,186, entitled Ductus Side-entry Jackets and Prosthetic Disorder Response Systems, are perivascular, or extraluminal, for use to partially constrict the substrate ductus from without, positioned within the anal canal where the ductus is not encircleable, a rectal stopper is endoluminal, intended to prevent leakage much less an uncontrollable loss of stool. Unlike the endothelia of vessels, the mucosal lining of the digestive and urinary tract, especially at the levels of native sphincters, is adapted for and tolerant to circumferential compression to effect secure closure.

Where a neoplasm, for example, necessitated a partial native sphincterectomy, once fully healed, this toughness is largely restored. Requiring placement in surrounding relation to the substrate ductus limits sphincteric-type choke servovalves to ductus which can be grasped by encirclement. By contrast, transluminal delivery to an intraluminal location offers minimal invasiveness and trauma with transluminal follow-up treatment if necessary. However, in relatively high force locations such as the rectum, the risk of migration for any device positioned within the lumen is considerable. Extraluminal stitching defeating the transluminal advantage, preservation of the transluminal benefit promotes antimigratory means such as barbs and endoscopically placed stitches.

The selection of intra- or extraluminal placement is dictated by the anatomy. Adaptation for complete closure also applies to the urothelium at the internal, or bladder neck, and external urethral sphincters but not to the urothelium lining the rest of the lower urinary tract, to include the ureters and urethra. Temporary placement during cryptosporidiosis or cyclosporiasis, self-limiting parasitic infections of the gut, for example, where urge sensation precedes forcible ejection immediately, and stool consists primarily of water, so that the internal and external sphincters cannot stop the peristaltically forced outflow, allows the patient to prevent the uncontrollable ejection long enough to reach a bathroom.

Absent such an aid, the condition restricts the patient to remain within immediate reach of a bathroom, disallowing normal activities. Expulsion in cryptosporidiosis would otherwise result in the release of highly contagious cyst- and in cyclosporiasis, oocyst-infected waste, quickly spread due to its watery consistency, demanding immediate removal and disinfection to avert the risk to public health. Where due to a war injury, for example, the native segment that would contain the sphincter is missing, an intraluminal stopper or an extraluminal electromagnetic sphincter can be used.

To prevent the passage of watery stool, an extraluminal valve sphincter is placed about a length of autologous tissue, likely gut best elongated through serial transverse enteroplasty or Bianchi longitudinal intestinal lengthening and tapering, or tissue engineered, tubulated to the correct diameter, used as a prosthesis. However, an extraluminal sphincter will probably prove no more able to stop the passage of highly watery stool than are normal native sphincters.

Other conditions that cause fecal incontinence are rectal hyposensitivity, where such a device can supplement electrical neuromodulation in the treatment of fecal incontinence associated with chronic constipation, involuntary encopresis, where young children involuntarily soil their underwear, and fecal leakage in an adult (see, for example, Mundet, L., Cabib, C., Ortega, O., Rofes, L., Tomsen, N., and 3 others 2019. “Defective Conduction of Anorectal Afferents is a Very Prevalent Pathophysiological Factor Associated to Fecal Incontinence in Women,” Journal of Neurogastroenterology and Motility 25(3): 423-435; Widmann, B., Galata, C., Warschkow, R., Beutner, U., Ogredici, O., and 4 others 2019. “Success and Complication Rates after Sacral Neuromodulation for Fecal Incontinence and Constipation: A Single-center Follow-up Study,” Journal of Neurogastroenterology and Motility 25(1): 159-170; Heywood, N. A., Pearson, J. S., Nicholson, J. E., Molyneux, C., Sharma, A., and 3 others 2018. “The Short-term Effects of Posterior Tibial Nerve Stimulation on Anorectal Physiology in Patients with Faecal Incontinence: A Single Centre Experience,” Therapeutic Advances in Gastroenterology 11:1756284818786111; Simillis, C., Lal, N., Qiu, S., Kontovounisios, C., Rasheed, S., Tan, E., and Tekkis, P. P. 2018. “Sacral Nerve Stimulation versus Percutaneous Tibial Nerve Stimulation for Faecal Incontinence: A Systematic Review and Meta-analysis,” International Journal of Colorectal Disease 33(5): 645-648; Saldana Ruiz, N. and Kaiser, A. M. 2017. “Fecal Incontinence-Challenges and Solutions,” World Journal of Gastroenterology 23(1): 11-24; Burgell, R. E. and Scott, S. M. 2012. “Rectal Hyposensitivity,” Journal of Neurogastroenterology and Motility 18(4): 373-384).

An anal stopper can comprise an inflatable insert made of a medical grade balloon material high in tensile strength, such as biaxially oriented stretched polyethylene terephthalate polyester. Once inserted, the device is inflated by squeezing a small bulb at the end of an air line connected to a unidirectional or check air valve with release by pulling on the bulb, causing it to deflate. If necessary, the deflated insert is coated with a lubricant, such as petrolatum, which to reduce if not dispel urge sensation due to pressure against rectal stretch sensors, may be wetted with a topical anesthetic. The bulb squeezed to inflate and pulled to remove the device is preferably continuous with the rest of the device.

In a reusable embodiment, absent a bidet, a separately carried bulb irrigator (anal douche, rectal bulb syringe) filled with a liquid antiseptic is used to wash away debris after the device is removed. Toward its lower surface, an anal canal stopper according to an alternative embodiment can incorporate a torsion spring-loaded hinged stainless steel damper in a stainless steel surround with damper-lid pull spring latch, which until pulled open by the patient, closes off the anus. In some cases, the device can be positioned to close off the rectum from the inferior or caudal end of the sigmoid colon.

### Lymphoid Neogenesis in Allografts

Pending the development of lymphoid neogenesis and tertiary lymphoid organ formation-suppressive drugs, the contribution to rejection of solid organ allografts with the participation of such drugs can also be targeted along the same accessory channels (see, for example, Hsiao, H. M., Li, W., Gelman, A. E., Krupnick, A. S., and Kreisel, D. 2016. “The Role of Lymphoid Neogenesis in Allografts,” American Journal of Transplantation 16(4): 1079-1085; Koenig, A. and Thaunat, O. 2016. “Lymphoid Neogenesis and Tertiary Lymphoid Organs in Transplanted Organs,” Online, Frontiers in Immunology (Lausanne, Switzerland) 7:646; Jing, F. and Choi, E. Y. 2016. “Potential of Cells and Cytokines/Chemokines to Regulate Tertiary Lymphoid Structures in Human Diseases,” Immune Network 16(5): 271-280; Jones, G. W., Hill, D. G., and Jones, S. A. 2016. “Understanding Immune Cells in Tertiary Lymphoid Organ Development: It Is All Starting to Come Together,” Online, Frontiers in Immunology (Lausanne, Switzerland) 7:401; Jones, G. W. and Jones, S. A. 2016. “Ectopic Lymphoid Follicles: Inducible Centres for Generating Antigen-specific Immune Responses Within Tissues,” Immunology 147(2): 141-151; Hughes, C. E., Benson, R. A., Bedaj, M., and Maffia, P. 2016. “Antigen-presenting Cells and Antigen Presentation in Tertiary Lymphoid Organs,” Online, Frontiers in Immunology (Lausanne, Switzerland) 7:481).

Infection by drug resistant pathogens following a compatible solid organ allograft in a patient necessarily immunosuppressed a common cause of failure, the direct targeting to the graft of antimicrobials (antivirals and antibiotics), anti-inflammatoires, and an anesthetic in higher than systemic concentration if necessary, with a surface port as common infusion point to side-entry jackets and/or side-entry connectors, which are less infection-prone than are conventional means, and the concurrent infusion through the same surface port of donor allogeneic chimerizing multipotent hematopoietic marrow-derived stem cells is likely to reduce the rejection rate.

Two essential methods are provided for targeting immunosuppressant, chemotherapeutic, or any other drug prepared in the form of a ferrofluid containing superparamagnetic iron oxide nanoparticles, or SPIONs, into a target organ. The first of these methods is to systemically circulate a lower dose of the drug or drugs than were it directly pipeline targeted to the target organ or tissue for the magnetically susceptible drug or drugs to be drawn from the blood into and thus concentrated within the targeted transplant or diseased organ or tissue by small electromagnets, patch or clasp electromagnets fastened thereto as described in U.S. Pat. No. 11,759,186. Using a jacket itself magnetized at a distance preceding the organ allows the main artery supplying the transplant or diseased organ to be likewise medicated, and if necessary, increased in the extent of drug extraction into the endothelium or urothelium prior to reaching the target.

Such is accomplished by increasing the distance from the target of the jacket feeding the drug or drugs and adding nonpiped electromagnetic jackets, adjusted to successively apply greater field strength normally to the lumen at intervals along the blood supply, thereby obtaining extension with uniformity of uptake. That is, using the first method, a ductus side-entry jacket is used not in lieu of a central line to directly pipe magnetically susceptible medication to the target but rather to eliminate the need for a conventional needle and line and allow the automatic infusion at controller programed intervals from the implant reservoir rather than by recalling a discharged patient for repeated insertion of a needle and line in the clinic with the risk of infection increased nosocomially and because the body surface port used is sealed from the environment.

The other method is to directly target the allograft or diseased organ by mechanical piping of the drug or drugs through the surface port or a reservoir into which it feeds and a side-entry jacket positioned proximally to the target along its primary blood supply, with no dose allowed into the systemnic circulation. Here too, a magnetized piped jacket and unpiped extension jackets allow the artery supplying the transplant or diseased organ to be likewise medicated.

These are the opposite poles in such treatment, and in practice, some intervening method whereby some less than or minimally injurious dose is circulated in both donor and recipient prior to harvesting or the recipient alone is treated thus as tailored to the individual patient. The foregoing should not be interpreted in a limiting sense as applying only to transplant or malignant organs and tissues but rather as applicable whenever a drug would best be targeted to the root, or site, of disease or lesions of disease.

### Transportation

Transplantation with direct recipient-to-donor organ bloodstream switching whereby recipient blood flow is continuous throughout the procedure during which his diseased organ is replaced with a healthy one with little meaningful deviation in blood pressure, much less a protracted state of no-flow, is a significant advantage made possible by ductus side-entry jackets modified to serve as vascular in-line valves. However, until direct side-by-side switching becomes standardized, the donor organ will continue to remain in hypothermic storage at an unnatural temperature commonly not sustained by a perfusion machine for up to six hours after incurring the shock of having been severed, resulting in considerable injury when normothermal perfusion is resumed.

Accordingly, where the prospect of switch technique is a reality, the importance of not removing the donor organ preoperatively but rather sustaining the donor as intact through intensive somatic support is of fundamental importance. For sudden compound vascular bypass, or sudden switched-organ transplantation where urgency dictates that procedural technicalities be kept to a minimum, the exchange of cells between donor and recipient to achieve an initial measure of mutual immune accommodation is manual. For metered compound vascular bypass, or sudden switched-organ transplantation where urgency is less intense and the optimization of matching is paramount, consideration should be given to the use of magnetized blood products and the positioning of patch electromagnets as described in U.S. Pat. No. 11,759,186 at points about the allograft to increase uptake prior to and during the transplant.

Thereafter, even when the administration of transplant maintenance medication is systemic, and when pipeline targeted, that fraction that is circulated, these continue to assist in the uptake of immunosuppressants, chemotherapeutics, and antimicrobials. The recipient should have been fitted with a surface port and subdermally positioned small flat pectoral reservoirs with outlet pumps or drip stopcocks controlled by an implanted microcontroller to follow a prescription-program.

Also assured is that a side-entry jacket has been positioned proximal to the proposed level along the blood supply of the dysfunctional or diseased target organ for placement of the allograft or lesioned native organ to draw the immunosuppressants and/or chemotherapeutics into the allograft or native organ to be targeted. As addressed in U.S. Pat. No. 11,389,171, entitled Integrated System for the Infixion and Retrieval of Implants, when an immunosuppressant, chemotherapeutic, or other drug would be injurious to other tissue as to best be restricted to the lesion or transplant, and magnetic uptake is not complete, a second side-entry jacket is positioned along the excurrent vessel or vessels to allow the direct delivery thereto of an innocuous reversal agent.

2b(2)(k). Surgical Chest Dome

Switch transplantation ordinarily eliminates the need for cardiopulmonary support under general anesthesia with the need for a mechanical ventilation mask or endotracheal intubation, the need to enter the chests of donor and recipient unavoidable. However, whenever the chest is entered, the thoracic viscera are exposed to the somewhat higher pressure of the surrounding air, causing the lungs to deflate and collapse. To allow ‘spontaneous’ ventilation by a deceased or genuinely spontaneous ventilation by a living donor as well as the recipient as preferred, it is necessary to eliminate a surgical pneumothorax.

The inexpert use of a ventilator or any other means of mechanical ventilatory support for that matter can result in serious damage to the lungs, and the elimination of mechanical ventilation eliminates a major source of serious complications. In most cases, the compound vascular bypass technique for organ transplantation, even of the heart and/or lungs, can be performed under regional or even local anesthesia, if necessary, with the aid of an anxiolytic, to allow spontaneous breathing.

FIG. 23A provides an overhead perspectival view of a surgical chest dome, FIG. 23B, a side-view with surrounding cast iron weight and foam base hidden behind the arm of the patient, FIG. 23C a closer side view, FIG. 23D, an upper surface (superior, cephalad, headward) view of the top side, and FIG. 23E, an upper surface (inferior, caudad, footward) view of the bottom surface of a surgical chest dome.

While oxygen and an aerosolized antimicrobial or steroid, for example, can be administered through the dome, and the air inside can be warmed, it is not intended as a kind of mechanical ventilator and not meant to supplant the use of conventional means in emergency situations, but rather a means to avoid both mechanical ventilation and surgical pneumothorax when the conscious patient under regional anesthesia is able to breathe normally or the unconscious patient or deceased organ donor can do so when assisted. Emergent, or emergency, intubation, for example, is a quite different and more aggressive way to maintain ventilation in a critically ill patient; for long-term maintenance, it should cause few if any complications.

When the patient is incapable of adequate spontaneous ventilation, its reinstatement is best by precisely selective stimulation of the respiratory center in the brainstem (which optogenetics will facilitate). The chest dome, addressed above in section 2a and below in section 5, entitled Description of the Drawing Figures, can also be used to facilitate spontaneous ventilation in a deceased donor upon removal of the heart or thyroid, for example. Ordinarily, to revive the cardiorespiratory centers in the brainstem following the loss of perfusion would be impossible; however, because as described in section 2a a deceased donor is placed on life support in advance of dying, perfusion is uninterrupted.

After direct respiratory center stimulation, next best is mechanical ventilation using high-flow nasal cannula oxygen therapy, which leaves the mouth of the patient free, thus allowing the patient to speak, eat, drink, and if an infant, eliminating the restraint on free movement imposed by apparatus on the face or in the mouth (see, for example, Entriken, C. and Pritts. T. A. 2021. “Perioperative Pulmonary Support of the Elderly,” Online, Current Geriatric Reports 10(4): 167-174; Beng Leong. L., Wei Ming, N., and Wei Feng. L. 2019. “High Flow Nasal Cannula Oxygen versus Noninvasive Ventilation in Adult Acute Respiratory Failure: A Systematic Review of Randomized-Controlled Trials,” European Journal of Emergency Medicine 26(1): 9-18; Bräunlich, J., Dellweg, D., Bastian, A., Budweiser, S., Randerath, W., and 9 others 2019. “Nasal High-flow versus Noninvasive Ventilation in Patients with Chronic Hypercapnic COPD,” International Journal of Chronic Obstructive Pulmonary Disease 14:1411-1421; Cheng. L. C., Chang, S. P., Wang, J. J., Hsiao, S. Y., Lai, C. C., and Chao, C. M. 2019. “The Impact of High-flow Nasal Cannula on the Outcome of Immunocompromised Patients with Acute Respiratory Failure: A Systematic Review and Meta-Analysis,” Medicina (Basel, Switzerland) 55(10). pii: E693; Kang. H., Zhao, Z., and Tong, Z. 2019. “Effect of High-flow Nasal Cannula Oxygen Therapy in Immunocompromised Subjects with Acute Respiratory Failure,” Respiratory Care November 19. pii: respcare.07205; Norkienė, I., d'Espiney, R., and Martin-Lazaro, J. F. 2019. “Effectiveness of High-flow Nasal Oxygen Therapy in Management of Acute Hypoxemic and Hypercapnic Respiratory Failure,” Acta Medica Lituanica (Vilnius, Lithuania) 26(1): 46-50; Lodeserto, F. J., Lettich, T. M., and Rezaie, S. R. 2018. “High-flow Nasal Cannula: Mechanisms of Action and Adult and Pediatric Indications,” Online, Cureus (Palo Alto, California) 10(11): e3639; Leeies, M., Flynn, E., Turgeon, A. F., Paunovic, B., Loewen, H., and 4 others 2017. “High-flow Oxygen via Nasal Cannulae in Patients with Acute Hypoxemic Respiratory Failure: A Systematic Review and Meta-analysis,” Online, BioMed Central Systematic Reviews (London, England) 6(1): 202; Parke. R. L., McGuinness, S. P., and Eccleston, M. L. 2011. “A Preliminary Randomized Controlled Trial to Assess Effectiveness of Nasal High-flow Oxygen in Intensive Care Patients,” Respiratory Care 56(3): 265-270).

Next best is mechanical ventilation using a mask (see, for example, Bishopp, A., Oakes. A., Antoine-Pitterson, P., Chakraborty, B., Comer, D., and Mukherjee, R. 2019. “The Preventive Effect of Hydrocolloid Dressings on Nasal Bridge Pressure Ulceration in Acute Non-invasive Ventilation,” Ulster Medical Journal 88(1): 17-20; Cai, J. Y., Zha, M. L., and Chen, H. L. 2019. “Use of a Hydrocolloid Dressing in the Prevention of Device-related Pressure Ulcers during Noninvasive Ventilation: A Meta-analysis of Randomized Controlled Trials,” Wound Management and Prevention (Malvern, Pennsylvania) 65(2): 30-38; Coudroy, R., Pham, T., Boissier, F., Robert, R., Frat. J. P., and Thille, A. W. 2019. “Is Immunosuppression Status a Risk Factor for Noninvasive Ventilation Failure in Patients with Acute Hypoxemnic Respiratory Failure? A Post Hoc Matched Analysis,” Annals of Intensive Care 9(1): 90; Karim, H. M. R. and Esquinas, A. M. 2019. “Autocycling during Noninvasive Positive Pressure Ventilation as a Factor for Severe Syncope: Exciting Observation, but Needs More Scrutiny,” Journal of Clinical Sleep Medicine (Darien, Illinois) 15(9): 1377; Mu. S., Howard, M. E., and Hannan, L. 2019. “Autocycling during Noninvasive Positive Pressure Ventilation Producing a Prolonged Severe Apnea and Syncope, Further Insights,” Journal of Clinical Sleep Medicine (Darien, Illinois) 15(9): 1379; Alqahtani, J. S. and AlAhmari, M. D. 2018. “Evidence Based Synthesis for Prevention of Noninvasive Ventilation Related Facial Pressure Ulcers,” Saudi Medical Journal 39(5): 443-452; Brill, A. K., Pickersgill, R., Moghal, M., Morrell, M. J., and Simonds, A. K. 2018. “Mask Pressure Effects on the Nasal Bridge during Short-term Noninvasive Ventilation,” European Respiratory Journal Open Research 4(2). pii: 00168-2017; de Picciotto, C., Dumenil. C., Auzel, O., Giraud, V., and Bonay, M. 2017. “Paradoxical Worsening of Hypoxemia in a Patient Treated by Noninvasive Positive Pressure Ventilation for Obesity Hypoventilation Syndrome with Concomitant Obstructive Sleep Apnea: A Case Report,” Online, BioMed Central Journal of Medical Case Reports (London, England) 11(1): 234.; Lemyze, M., Mallat. J., Nigeon, O., Barrailler, S., Pepy, F., and 5 others 2013. “Rescue Therapy by Switching to Total Face Mask after Failure of Face Mask-delivered Noninvasive Ventilation in Do-not-intubate Patients in Acute Respiratory Failure,” Critical Care Medicine 41(2): 481-488; Gay, P. C. 2009. “Complications of Noninvasive Ventilation in Acute Care,” Respiratory Care 54(2): 246-258, Online pdf at http://rc.rejournal.com/content/54/2/246/tab-pdf; Honrubia. T., Garcia Lopez, F. J., Franco, N., Mas, M., Guevara, M., and 4 others 2005. “Noninvasive vs Conventional Mechanical Ventilation in Acute Respiratory Failure: A Multicenter, Randomized Controlled Trial,” Chest 128(6): 3916-3924).

Most susceptible to complications is endotracheal intubation, which for present purposes, risks tracheal and intrapulmonary injury to the lungs of a deceased prospective organ donor no less than to a living patient. The risk is less with high-frequency jet ventilation, however, this still requires the insertion of a tube down the trachea (see, for example, Fong. K. M., Au, S. Y., and Ng. G. W. Y. 2019. “Preoxygenation before Intubation in Adult Patients with Acute Hypoxemic Respiratory Failure: A Network Meta-analysis of Randomized Trials,” Critical Care (London, England) 23(1): 319; Matettore, A., Ramnarayan, P., Jones. A., Randle, E., Lutman, D., O'Connor, M., and Chigaru, L. 2019. “Adverse Tracheal Intubation-associated Events in Pediatric Patients at Nonspecialist Centers: A Multicenter Prospective Observational Study,” Pediatric Critical Care Medicine 20(6): 518-526; Park, C. 2019. “Risk Factors Associated with Inpatient Cardiac Arrest during Emergency Endotracheal Intubation at General Wards,” Acute and Critical Care (Seoul, South Korea) 34(3): 212-218; Sanders. R. Jr., Edwards, L., and Nishisaki, A. 2019. “Tracheal Intubations for Critically Ill Children Outside Specialized Centers in the United Kingdom-Patient, Provider, Practice Factors, and Adverse Events,” Pediatric Critical Care Medicine 20(6): 572-573; Santos, A. K., Silveira, J., Neves, V. C., Zotz, T. G. G., Motter, A. A., and Andreazza, M. G. 2019. “Atelectasis and Lung Changes in Preterm Neonates in the Neonatal Period: A Blind Radiological Report and Clinical Findings,” Revista Brasileira de Terapia Intensiva (Brazilian Journal of Intensive Care, Rio de Janeiro, Brazil) 31(3): 347-353; Tikka, T. and Hilmi, O. J. 2019. “Upper Airway Tract Complications of Endotracheal Intubation,” British Journal of Hospital Medicine (London, England 80(8): 441-447; Cohn, E. C., Robertson, T. S., Scott, S. A., Finley, A. M., Huang. R., and Miles, D. K. 2018. “Extubation Failure and Tracheostomy Placement in Children with Acute Neurocritical Illness,” Neurocritical Care 28(1): 83-92; Dominguez, M. C. and Alvares, B. R. 2018. “Pulmonary Atelectasis in Newboms with Clinically Treatable Diseases Who are on Mechanical Ventilation: Clinical and Radiological Aspects,” Radiologia Brasileira 51(1): 20-25; Li, S., Hsieh, T. C., Rehder, K. J., Nett, S., Kamat, P., and 35 others 2018. “Frequency of Desaturation and Association with Hemodynamic Adverse Events during Tracheal Intubations in PICUs,” Pediatric Critical Care Medicine 19(1): e41-e50; Silva-Cruz, A. L., Velarde-Jacay, K., Carreazo, N. Y., Escalante-Kanashiro, R. 2018. “Risk Factors for Extubation Failure in the Intensive Care Unit,” Revista Brasileira de Terapia Intensiva (Brazilian Journal of Intensive Care, Rio de Janeiro, Brazil) 30(3): 294-300; Aykac, K., Ozsurekci, Y., and Tanir Basaranoglu, S. 2017. “Future Directions and Molecular Basis of Ventilator Associated Pneumonia,” Canadian Respiratory Journal 2017:2614602; Parker, M. M., Nuthall, G., Brown, C. 3rd, Biagas, K., Napolitano, N., and 34 others with 9 collaborators 2017. “Relationship between Adverse Tracheal Intubation Associated Events and PICU Outcomes,” Pediatric Critical Care Medicine 18(4): 310-318; Ricard, J. D. 2016. “Hazards of Intubation in the ICU: Role of Nasal High Flow Oxygen Therapy for Preoxygenation and Apneic Oxygenation to Prevent Desaturation,” Minerva Anestesiologica 82(10): 1098-1106; Shiima, Y., Berg. R. A., Bogner, H. R., Morales, K. H., Nadkami, V. M., and Nishisaki, A, with 28 collaborators 2016. “Cardiac Arrests Associated with Tracheal Intubations in PICUs: A Multicenter Cohort Study,” Critical Care Medicine 44(9): 1675-1682; Siddiqui, S. S., Janarthanan, S., Harish, M. M., Chaudhari, H., Prabu, R. N., Divatia, J. V., and Kulkami, A. P. 2016. “Complications of Tracheal Intubation in Critically Ill Pediatric Cancer Patients,” Indian Journal of Critical Care Medicine 20(7): 409-411; Weinberg, J. A., Stevens, L. R., Goslar, P. W., Thompson, T. M., Sanford, J. L., and Petersen, S. R. 2016. “Risk Factors for Extubation Failure at a Level I Trauma Center: Does the Specialty of the Intensivist Matter?,” Trauma Surgery and Acute Care Open 1(1): e000052; Lapinsky, S. E. 2015. “Endotracheal Intubation in the ICU,” Online, BioMed Central Critical Care (London, England) 19:258; Law, J. A., Morris, I. R., Brousseau, P. A., de la Ronde, S., and Milne, A. D. 2015. “The Incidence, Success Rate, and Complications of Awake Tracheal Intubation in 1,554 Patients over 12 years: An Historical Cohort Study,” Canadian Journal of Anaesthesia 62(7): 736-744; Rehder, K. J., Giuliano, J. S. Jr., Napolitano, N., Tumer, D. A., Nuthall, G., Nadkarni, V. M., and Nishisaki, A., with 27 collaborators 2015. “Increased Occurrence of Tracheal Intubation-associated Events during Nights and Weekends in the PICU,” Critical Care Medicine 43(12): 2668-2674; Tarquinio, K. M., Howell, J. D., Montgomery, V., Turner, D. A., Hsing. D. D., and 5 others with 11 collaborators 2015. “Current Medication Practice and Tracheal Intubation Safety Outcomes from a Prospective Multicenter Observational Cohort Study,” Pediatric Critical Care Medicine 16(3): 210-218; Bassi, G. L., Ferrer, M., Marti, J. D., Comaru, T., and Torres, A. 2014. “Ventilator-associated Pneumonia,” Seminars in Respiratory and Critical Care Medicine 35(4): 469-481; Hsu, C. W. and Sun, S. F. 2014. “Iatrogenic Pneumothorax Related to Mechanical Ventilation,” World Journal of Critical Care Medicine 3(1): 8-14; Rashid, A. M., Williams, C., Noble. J., Rashid, O. M., Takabe, K., and Anand, R. J. 2012. “Pneumothorax, an Underappreciated Complication with an Airway Exchange Catheter,” Journal of Thoracic Disease 4(6): 659-662; Hatch, L. D., Grubb, P. H., Lea, A. S., Walsh, W. F., Markham, M. H., and 4 others 2016. “Endotracheal Intubation in Neonates: A Prospective Study of Adverse Safety Events in 162 Infants,” Journal of Pediatrics 168:62-6.e6; Kulkarni, A. P. and Agarwal. V. 2008. “Extubation Failure in Intensive Care Unit: Predictors and Management,” Indian Journal of Critical Care Medicine 12(1): 1-9; Rivera, R. and Tibballs, J. 1992. “Complications of Endotracheal Intubation and Mechanical Ventilation in Infants and Children,” Critical Care Medicine 20(2): 193-199; additional references cited below in this section).

Following a generally trouble-free initial period, all produce adverse sequelae in proportion to the duration of use. Less desirable, especially when prolonged, is extrapulmonary direct oxygenation of the blood by a cardiopulmonary bypass machine, of which the blood pump imposes much damage on red blood cells. All are associated with numerous complications, some serious and lasting, so that, when possible, spontaneous breathing should be preferred.

A surgical chest dome is an airtight positive pressure glovebox designed for open chest surgery of any kind, but in particular for a switch heart or thyroid transplant, or carotid endarterectomy performed as described in section 1b(2)(d), for example, which complements the elimination of cardiopulmonary support or extracorporeal membrane oxygenation, cardioplegia, cross-clamping, general anesthesia, and in many cases, the need for mechanical ventilation, by maintaining equal pressures outside and inside the chest, thus allowing spontaneous ventilation even when the lungs are exposed.

Elimination of the foregoing leaves only mechanical rather than spontaneous ventilation as a source of complications, and the surgical chest dome is intended to eliminate this last source. The chest dome stands clear of while enveloping the thorax with a clear transparent plastic shell having flexible entry points which allow the operator free access to the operating field from any angle and extends downward into flush contact with the operating table all around providing openings on either side to pass through the arms. The chest dome is intended to overcome this last impediment of compelling the use mechanical ventilation or oxygenation during cardiothoracic surgery where the single lung technique cannot be used.

Use of a chest dome requires that the airway be intact—it is not applicable to a tracheostomy, for example. By the same token, by leaving the trachea empty, its use during and following an esophagectomy, especially in a neonate, infant, or young child reduces the risk for injury to the trachea as compared to the use of endotracheal intubation. The chest dome is intended to allow the patient capable of spontaneous ventilation, that is, without respiratory failure of a serious disorder of the lungs per se as affects the patient in a lung or heart-lung transplantation, for example, to avoid the need for mechanical ventilation, which even with the use of a mask rather than intubation, which presents its own risks, can pose other complications, the more so as the length of time on the apparatus is increased.

The prolonged mechanical ventilation to which a deceased prospective organ donor, or a neonate, or an infant is subjected for correction or replacement of a congenitally defective heart is favorably eliminated through the use of a chest dome, which also affords greater freedom of movement than does intubation (see, for example, Alrddadi, S. M., Morsy, M. M., Albakri, J. K., Mohammed, M. A., Alnajjar, G. A., and 15 others 2019. “Risk Factors for Prolonged Mechanical Ventilation after Surgical Repair of Congenital Heart Disease. Experience from a Single Cardiac Center,” Saudi Medical Journal 40(4): 367-371; Nasser, B. A., Mesned, A. R., Mohamad, T., and Kabbani. M. S. 2018. “Incidence and Causes of Prolonged Mechanical Ventilation in Children with Down Syndrome Undergoing Cardiac Surgery,” Journal of the Saudi Heart Association 30(3): 247-253; Parmar, D., Lakhia. K., Garg. P., Patel, K., Shah, R., and 3 others 2017. “Risk Factors for Delayed Extubation after Ventricular Septal Defect Closure: A Prospective Observational Study,” Brazilian Journal of Cardiovascular Surgery 32(4): 276-282; Chin, T., Kushner, B., Dersch-Mills, D., and Zuege, D. J. 2016. “Antibiotic Utilization Patterns in Patients with Ventilator-associated Pneumonia: A Canadian Context,” Canadian Journal of Infectious Diseases and Medical Microbiology 2016:3702625; Tabib. A., Abrishami, S. E., Mahdavi, M., Mortezaeian, H., and Totonchi, Z. 2016. “Predictors of Prolonged Mechanical Ventilation in Pediatric Patients after Cardiac Surgery for Congenital Heart Disease,” Research in Cardiovascular Medicine 5(3): e30391; Hsu, C. W. and Sun, S. F. 2014. “Iatrogenic Pneumothorax Related to Mechanical Ventilation,” World Journal of Critical Care Medicine 3(1): 8-14; Eachempati, S. R., Hydo, L. J., Shou, J., and Barie, P. S. 2009. “Does De-escalation of Antibiotic Therapy for Ventilator-associated Pneumonia Affect the Likelihood of Recurrent Pneumonia or Mortality in Critically Ill Surgical Patients?,” Journal of Trauma 66(5): 1343-1348; Eagye, K. J., Nicolau, D. P., and Kuti, J. L. 2009. “Impact of Superinfection on Hospital Length of Stay and Costs in Patients with Ventilator-associated Pneumonia,” Seminars in Respiratory and Critical Care Medicine 30(1): 116-123; Traiber. C., Piva, J. P., Fritsher, C. C., Garcia, P. C., Lago, P. M., and 5 others 2009. “Profile and Consequences of Children Requiring Prolonged Mechanical Ventilation in Three Brazilian Pediatric Intensive Care Units,” Pediatric Critical Care Medicine 10(3): 375-380; Trouillet, J. L., Combes, A., Vaissier, E., Luyt. C. E., Ouattara, A., Pavie. A., and Chastre, J. 2009. “Prolonged Mechanical Ventilation after Cardiac Surgery: Outcome and Predictors,” Journal of Thoracic and Cardiovascular Surgery 138(4): 948-953; Kendirli, T., Kavaz, A., Yalaki, Z., Ozturk Hişmi. B., Derelli, E., and Ince, E. 2006. “Mechanical Ventilation in Children” Turkish Journal of Pediatrics 48(4): 323-327; Macintyre, N. R. 2005. “Ventilator-associated Pneumonia: The Role of Ventilator Management Strategies,” Respiratory Care 50(6): 766-773; Cheifetz, I. M. 2003. “Invasive and Noninvasive Pediatric Mechanical Ventilation,” Respiratory Care 48(4): 442-458; Pierson, D. J. 1990. “Complications Associated with Mechanical Ventilation,” Critical Care Clinics 6(3): 711-724; Strieter, R. M. and Lynch, J. P. 3rd. 1988. “Complications in the Ventilated Patient,” Clinics in Chest Medicine 9(1): 127-139; Wang, G. C., Kao, H. A., Hwang, F. Y., Ho, M. Y., Hsu, C. H., and Hung, H. Y. 1991. “Complications in the Use of Mechanical Ventilator in Newborns: One Year's Experience,” (in Chinese with English abstract at Pubmed), Zhonghua Min Guo Xiao Er Ke Yi Xue Hui Za Zhi (Acta Paediatrica Sinica) 32(4): 227-232; additional references cited above in this section).

In any patient, but especially in neonates and infants, a chest dome eliminates the risk of lung damaging excessive pressure with mechanical ventilation, and leaving the airway empty, facilitates and reduces the risk of surgical trauma during esophageal surgery (Toyama, H., Saito, K., Takei. Y., Saito, K., Fujimine, T., and 5 others 2016. “Perioperative Management of Esophagectomy in a Patient who Previously Underwent Bilateral Lung Transplantation,” Journal of Anesthesia Clinical Reports (Heidelberg, Germany) 2(1): 15). Nasal, mask, and endotracheal tube delivery all harass the conscious infant.

Addressed above in section 2a, entitled Background, General, in comatose patients with intact brainstem, improvement in spontaneous respiration when irregular or too rapid, for example, is best achieved by direct stimulation of the respiratory center. This issue pertains not only to the treatment of patients undergoing a surgical procedure for whom an acceptable method will not exchange neurological for ventilator-associated complications, but also to the sustainment of a deceased prospective organ donor for whom such complication will usually not be as serious but with whom the preservation of ‘normal’ cardiorespiratory and other bodily functions is central for maintaining all the organs in a condition acceptable for transplantation.

The ability to reinstate and sustain spontaneous respiration at a normal rate with normal tidal volume averts complications associated with mechanical ventilation, to include those related to intubation and extubation of the endotracheal tube (which applies whether the problem is primarily pulmonary), infection, increase in alveolar/capillary permeability by overdistension of the lungs, or volutrauma, recruitment/derecruitment of collapsed alveoli, or atelectrauma, activation of inflammatory processes, or biotrauma, can have significant effects on other organs and organic systems, and can be a significant factor contributing to the increase of morbidity and mortality in the critically ill (loosely quoted from Drašković, B. and Rakic, G. 2011. “Complications of Mechanical Ventilation,” (in Serbian with English abstract at Pubmed), Srpski Arhiv za Celokupno Lekarstvo (Serbian Archives of Medicine) 139(9-10): 685-692; (see also, Bates, J. H. T., Gaver, D. P., Habashi, N. M., and Nieman, G. F., 2020. “Atelectrauma versus Volutrauma: A Tale of Two Time-constants,” Online, Critical Care Explorations (Philadelphia, Pennsylvania) 2(12): e0299; Gualdi, L. P., Cabral. E. E., and 4 others 2019. “Chest Wall Volume and Asynchrony in Stroke and Parkinson's Disease Subjects: A Case-control Study,” Online, PLoS [Public Library of Science] One 14(5): e0216641; Gupta, E., Hossen, S., Grigsby, M. R., Herrera, P., Roldan, R., and 7 others with 20 collaborators 2019. “Risk factors for the Development of Acute Respiratory Distress Syndrome in Mechanically Ventilated Adults in Peru: A Multicenter Observational Study,” Critical Care 23(1): 398; Silva, P. L., Ball, L., Rocco, P. R. M., and Pelosi, P. 2019. “Power to Mechanical Power to Minimize Ventilator-induced Lung Injury?,” Intensive Care Medicine Experimental (Heidelberg, Germany) 7 (Supplement 1): 38; Cipulli. F., Vasques. F., Duscio, E., Romitti, F., Quintel, M. and Gattinoni, M. 2018. “Atelectrauma or Volutrauma: The Dilemma,” Online, Journal of Thoracic Disease (Hong Kong, China) 10(3): 1258-1264; Gattinoni. L., Quintel. M., and Marini, J. J. 2018. “Volutrauma and Atelectrauma: Which is Worse?,” Online, Critical Care (London, England) 22(1): 264; Lima, I. N. D. F., Cruz, F. F., Ball, L., Rocco, P. R. M., and Pelosi, P. 2018. “Ventilator-induced Lung Injury during Controlled Ventilation in Patients with Acute Respiratory Distress Syndrome: Less is Probably Better,” Expert Review of Respiratory Medicine 12(5): 403-414; Florêncio. R. B., da Nobrega, A. J. S., Gattinoni, L., Marini, J. J., Collino, F., Maiolo, G., Rapetti, F., and 3 others 2017. “The Future of Mechanical Ventilation: Lessons from the Present and the Past,” Critical Care 21(1): 183; Gattinoni, L., Tonetti, T., Cressoni, M., Cadringher, P., Herrmann, P., and 7 others 2016. “Ventilator-related Causes of Lung Injury: The Mechanical Power,” Intensive Care Medicine 42(10): 1567-1575; Guldner, A., Braune, A., Ball. L., Silva, P. L., Samary, C., and 12 others 2016. “Comparative Effects of Volutrauma and Atelectrauma on Lung Inflammation in Experimental Acute Respiratory Distress Syndrome,” Online, Critical Care Medicine (Philadelphia, Pennsylvania) 44(9): e854-e865; Parreira, V. F., Vieira, D. S., Myrrha. M. A., Pessoa, I. M., Lage, S. M., and Britto. R. R. 2012. “Optoelectronic Plethysmography: A Review of the Literature,” Revista Brasileira de Fisioterapia (Brazilian Journal of Physical Therapy, São Carlos, São Paulo, Brazil) 16(6): 439-453; Miller, J. D. and Carlo, W. A. 2008. “Pulmonary Complications of Mechanical Ventilation in Neonates,” Clinical Perinatology 35(1): 273-281. x-xi; Namendys-Silva, S. A. and Posadas-Calleja, J. G. 2005. “Ventilator Associated Acute Lung Injury,” (Spanish with English abstract at Pubmed), Revista de Investigación Clinica (Journal of Clinical Investigation) 57(3): 473-480); Howard, R. S., Rudd, A. G., Wolfe, C. D., and Williams, A. J. 2001. “Pathophysiological and Clinical Aspects of Breathing after Stroke,” Postgraduate Medical Journal 77(913): 700-702: Leigh, R. J. and Shaw, D. A. 1976. “Rapid Regular Respiration in Unconscious Patients,” Archives of Neurology 33(5): 356-361).

Expanding the zone of application for spontaneous ventilation with the aid of a surgical chest dome as addressed above in section 2b(2)(k) and if necessary, brainstem stimulation, mechanical ventilation is usually tied to general anesthesia, which the switch method for transplantation and use of a surgical chest dome should eliminate often. Mechanical ventilation can cause complications when used to support surgery other than in the chest, and can both cause or exacerbate adverse neurocognitive function (see, for example, Fernandes, A., Rodrigues, J., Lages, P., Lança, S., Mendes, P.5., and 7 others 2019. “Root Causes and Outcomes of Postoperative Pulmonary Complications after Abdominal Surgery: A Retrospective Observational Cohort Study,” Patient Safety in Surgery 13:40; Yu, J., Park, J. Y., Kim, D. H., Kim, S., Hwang, J. H., Seo. H., and Kim, Y. K. 2019. “Incidence and Risk Factors of Pulmonary Complications after Robot-assisted Laparoscopic Prostatectomy: A Retrospective Observational Analysis of 2208 Patients at a Large Single Center,” Online, MDPI [Multidisciplinary Digital Publishing Institute] Journal of Clinical Medicine (Basel, Switzerland) 8(10). pii: E1509; Turon, M., Fernandez-Gonzalo, S., de Haro. C., Magrans, R., Lopez-Aguilar, J., and Blanch L. 2018. “Mechanisms Involved in Brain Dysfunction in Mechanically Ventilated Critically Ill Patients: Implications and Therapeutics,” Annals of Translational Medicine 6(2): 30; Bruni, A., Garofalo, E., Pelaia. C., Longhini. F., and Navalesi, P. 2017. “Mechanical Ventilation in Brain Injured Patients: Seeing the Forest for the Trees,” Journal of Thoracic Disease 9(10): 3483-3487; Bulic. D., Bennett, M., Rodgers, H., Nourse, M., Rubie, P., Looi, J. C., and Van Haren, F. 2017. “Delirium after Mechanical Ventilation in Intensive Care Units: The Cognitive and Psychosocial Assessment (CAPA) Study Protocol,” Journal of Medical Internet Research, Research Protocols 6(2): e31; Fernandez-Bustamante, A., Frendl. G., Sprung, J., Kor, D. J., Subramaniam, B., and 10 others 2017. “Postoperative Pulmonary Complications, Early Mortality, and Hospital Stay following Noncardiothoracic Surgery: A Multicenter Study by the Perioperative Research Network Investigators,” Journal of the American Medical Association Surgery 152(2): 157-166; LAS VEGAS Investigators with 492 collaborators 2017. “Epidemiology, Practice of Ventilation and Outcome for Patients at Increased Risk of Postoperative Pulmonary Complications: LAS VEGAS—An Observational Study in 29 Countries,” European Journal of Anaesthesiology 34(8): 492-507; Na. B., Zhang, H., Wang, G., Dai. L., and Xia, G. 2017. “The Effect of Mechanical Ventilation on TASK-1 [TWIK (K(2P), or two pore domain potassium channel, subfamily)-related acid-sensitive potassium channel 1] Expression in the Brain in a Rat Model,” Canadian Respiratory Journal 2017:8530352; Koutsoukou, A., Katsiari, M., Orfanos, S. E., Kotanidou, A., Daganou, M., and 3 others 2016. “Respiratory Mechanics in Brain Injury: A Review,” World Journal of Critical Care Medicine 5(1): 65-73; Coppola, S., Froio, S., and Chiumello, D. 2014. “Protective Lung Ventilation during General Anesthesia: Is There Any Evidence?,” Critical Care 18(2): 210).

In addition to minimizing injury to the donor and recipient hearts, this approach reduces the complexity of heart transplantation accomplished by conventional means. “Postoperative pulmonary complications are a leading cause of morbidity and mortality after cardiac surgery” (Nguyen, L. S., Merzoug, M., Estagnasie, P., Brusset. A., Law Koune, J. D., and 5 others 2017. “Low Tidal Volume Mechanical Ventilation against No Ventilation during Cardiopulmonary Bypass Heart Surgery (MECANO) [undefined/MECHANical Oxygenation?]: Study Protocol for a Randomized Controlled Trial,” Online, BioMed Central Trials (London, England) 18(1): 582)(see also Lagier, D., Fischer, F., Fornier, W., Huynh, T. M., Cholley, B., and 20 others 2019. “Effect of Open-lung vs Conventional Perioperative Ventilation Strategies on Postoperative Pulmonary Complications after On-pump Cardiac Surgery: The PROVECS [perioperative open-lung protective ventilation in cardiac surgery] Randomized Clinical Trial,” Intensive Care Medicine 45(10): 1401-1412; Lagier, D., Fischer, F., Formier, W., Fellahi, J. L., Colson, P., and 4 others with 32 collaborators 2018. “A Perioperative Surgeon-controlled Open-lung Approach versus Conventional Protective Ventilation with Low Positive End-expiratory Pressure in Cardiac Surgery with Cardiopulmonary Bypass (PROVECS): Study Protocol for a Randomized Controlled Trial,” Online, BioMed Central Trials (London, England) 19(1): 624; Wang, Y. C., Huang, C. H., and Tu. Y. K. 2018. “Effects of Positive Airway Pressure and Mechanical Ventilation of the Lungs during Cardiopulmonary Bypass on Pulmonary Adverse Events after Cardiac Surgery: A Systematic Review and Meta-analysis,” Journal of Cardiothoracic and Vascular Anesthesia 32(2): 748-759).

Accordingly, the chest dome is intended to allow spontaneous ventilation during entry into the chest. As such, it enables the use of regional if not local rather than general anesthesia, the avoidance of cardiopulmonary bypass, cardioplegia, and vascular clamping during heart, lung, or thyroid surgery, for example. In so doing, it complements the sudden and metered switch methods for transplantation of the heart and/or a second heart. In many cases, it can eliminate the need for mechanical ventilation.

This when the adverse consequences using conventional methods fail to eliminate ischemia, blood cell damage, lung injury, and the risk of cognitive impairment. The elimination of ischemia may reduce the likelihood of cardiac allograft vasculopathy that will result in graft failure, and the elimination of general rather than regional anesthesia during cardiac surgery, to include transplantation can be a significant determinant in postoperative survival.

Concerning these complexities, numerous issues remain to be resolved (see, for example, Douville, N. J., Jewell, E. S., Duggal, N., Blank, R., Kheterpal. S., Engoren, M. C., and Mathis, M. R. 2020. “Association of Intraoperative Ventilator Management with Postoperative Oxygenation, Pulmonary Complications, and Mortality,” Anesthesia and Analgesia 2020 130(1): 165-175; Mathis, M. R., Duggal, N. M., Likosky, D. S., Haft, J. W., Douville, N. J., and 9 others 2019. “Intraoperative Mechanical Ventilation and Postoperative Pulmonary Complications after Cardiac Surgery,” Anesthesiology 131(5): 1046-1062; Bignami, E., Di Lullo, A., Saglietti, F., Guarnieri, M., Pota. V., and 5 others 2019. “Routine Practice in Mechanical Ventilation in Cardiac Surgery in Italy,” Journal of Thoracic Disease 11(4): 1571-1579; Zhang, M. Q., Liao, Y. Q., Yu, H., Li, X. F., Feng. L., Yang, X. Y., and Yu, H. 2019. “Ventilation Strategies with Different Inhaled Oxygen Concentration during Cardiopulmonary Bypass in Cardiac Surgery (VONTCPB) [Ventilation Oxygen conceNTration CardioPulmonary Bypass]: Study Protocol for a Randomized Controlled Trial,” Online, BioMed Central Trials (London, England) 20(1): 254; Bignami, E., Saglietti, F., and Di Lullo, A. 2018. “Mechanical Ventilation Management during Cardiothoracic Surgery: An Open Challenge,” Annals of Translational Medicine 6(19): 380; Lellouche, F., Delorme, M., Bussières, J., and Ouattara, A. 2015. “Perioperative Ventilatory Strategies in Cardiac Surgery,” Best Practice and Research. Clinical Anesthesiology 29(3): 381-395; Serpa Neto, A., Schultz, M. J., and Gama de Abreu, M. 2015. “Intraoperative Ventilation Strategies to Prevent Postoperative Pulmonary Complications: Systematic Review, Meta-analysis, and Trial Sequential Analysis,” Best Practice and Research. Clinical Anesthesiology 29(3): 331-340).

2b(2)(1). Compound Vascular Bypass Transplantation-Associated and Unassociated Anastomosis Bypasses

Representing the close to ending stage in a compound vascular bypass solid organ transplant, the anastomotic bypasses shown in FIGS. 22B thru 22E, 32F, and 35F incorporate vascular servovalves. Once the graft organ is implanted, the upper of both arterial and venous valves can be replaced with a solenoid driven valve in the upstream, or higher position as shown, and the lower valves can be replaced by simple, or fixed, side-entry diversion jackets. Only the upstream valves are furnished with druglines, or accessory channels, through which the fully implanted prosthetic disorder response system can deliver drugs in accordance with the system prescription-program.

Such simple valve and tubing assemblies can be provided independently as distinct products in an assortment of sizes unrelated to analogous uses addressed herein. When the upper valve closes off flow through the anastomotic segment to flow around the anastomosis through the bypass tube, drug delivery into the anastomotic segment is through the lower accessory channel, part number 8, at the bottom of the distal tip of the diversion chute in FIG. 2. Blood diverted about the anastomosis, the drug is released into the anastomotic segment without being immediately washed away. Occasional temporary relief from the flowing through of blood can sometimes afford a brief period to medicate and facilitate healing, such as were an anti-inflammatory or immunosuppressive released therein.

At the same time, the upper accessory channel 8′ can be used to release medication into the blood flowing through the bypass. Used in the vascular tree, one likely agent will be an anticoagulant, whereas in the urinary tract, it will be a crystal solvent. With numerous valves and implanted drug reservoir outlet motors, the simultaneous coordinated timing of drug release with the operation of the servovalves is assured by the master control microprocessor of the prosthetic disorder response system.

With one or a smaller number of prosthetic bypasses, for example, the controller can be a microcontroller rather than a master control microprocessor as is required for a more complex procedure such as an organ transplant. Bypasses such as those shown in FIGS. 22B thru 22E, 32D, and 32F also have intracorporeal, or transplantation-unrelated applications in general surgery. For example, a segment of an artery avulsed in combat or a traffic collision requires to be restored as soon after tourniqueted as possible. Conventionally, this is accomplished by taking a length of a vessel from elsewhere in the body.

FIG. 22F shows a tuned shunt, which is structurally the same as an anastomosis bypass of which the chute is controlled by a servovalve to allow the flow-thru cross-section to be continuously variable. It can then be adjusted by the implanted disorder response system in response to sensor inputs indicating adjustment as specified in the prescription-program. This is, however, exceptional, sudden repositioning of the chute from full open to fully closed or the reverse usual. Tuned shunts generally serve to control the tapped off output of a hypertensive vessel to another larger vessel and therefore always connect different vessels, whereas anastomosis bypasses are usually positioned between the stumps of one and the same vessel or ductus of the same kind as in a ureteroureteral or femorofemoral shunt where one of a paired ductus is lacking.

However, to do so not only constitutes a preliminary operation subject to the usual complications but can lead to atrophy. Harvesting an autologous internal mammary artery to replace an obstructed coronary artery where collateral circulation would not continue perfusion, for example, can lead to atrophy of the ipsilateral breast. A prosthesis also offers advantages. It need not be provided with a blood supply and drainage possibly necessitating a pedicle, does not provide but then also does not require endothelial function, and is oblivious to infection, rejection, and malignancy.

Moreover, if metal or coated or impregnated with metal, the prosthesis can be warmed by placement in a radiofrequency alternating magnetic field for the purpose of sterilizing or using warmth to accelerate healing in the adjacent tissue. Anastomosis can be vascular, intestinal, or nervous, the latter limited to transection without the loss of a segment unless the crushed or avulsed segment can be obtained from a donor maintained on life support. Conventional means include nerve conduits and nerve allografts, usually taken from elsewhere in the body where function is subcritical.

For use to protect an autologous anastomosis along a vessel, a nerve, or a peristaltic ductus, such as the gut, which also exhibits segmentation, or a ureter, or a gamete-moving duct, which had required surgical transection or had been divided by trauma, the arrangement shown in FIG. 22B can be modified to incorporate less costly parts than in a transplant and can be made available as a separate and distinct product consisting of a solenoid-driven entry or inlet valve, a length of tubing, and a side-entry diversion jacket. The tubing is fused to the valve side stem outlet and cut to the proper length to circle about the anastomosis for input into the diversion jacket.

Attachment of the free end of the tubing to the side stem outlet of the diversion jacket for placement upstream to the anastomosis is by a tight scored surface to scored surface friction fit and a strong adhesive such as one of those cited below. Also needed are lengths of catheteric tubing to serve as druglines leading from a microcontroller and drug reservoirs placed in the pectoral region which are led to the drug release points at the top of the side stem and below the diversion chute of the entry valve. The microcontroller is programmed by medical specialists and a prescription-programmer employed by the maker to switch between anastomosis flow-through and bypass with the release of drugs as will best treat the specific patient.

As opposed to FIG. 22C wherein the ends of the ductus stumps ere long enough to have been directly apposed and anastomosed, FIG. 22D shows the interpositioning of a synthetic replacement segment for a length of a vessel or glandular ductus, for example, which had been missing anomalously, resected as diseased, or avulsed or crushed as the result of trauma. In FIGS. 22C thru 22F, the continuously variable apportionment of flow between either of the two outlet passages responsive to pressure sensor feedback to the implanted response system controller necessitates regulation by a servovalve.

Lacking valve function, the segments interposed along the ductus in FIGS. 22D and 22E cannot be secured in position with either stump end connectors as shown in FIGS. 31A thru 31C, or inline coupling jackets as shown in FIGS. 36A 36E, and 37B or for that matter with any of several historical vessel end-connectors of little practical significance, for example. Rather, for bistable control, that is, the ability to switch all flow through either outlet passage, a solenoid-driven upper vascular valve with drugline, or accessory channel, a length of tubing which can be cut to size, and a lower side-entry diversion jacket are required.

However, most practical applications necessitating the continuously variable apportionment of flow between either of two outlet passages, the inlet valve will almost always be a servovalve. Along a peristaltic ductus such as the gut or a ureter, provided the interposed segment is short enough and positioned close enough to the outlet so that the contents will be propelled out through the end, the interposed segment can be a length of synthetic tubing. In FIGS. 22C thru 22E, the implanted automatic disorder control system can use drugline 8 to directly target medication into the bypass and/or drugline 8′ to target medication into the undiverted line.

The disorder response system coordinates the apportionment of flow and the release of medication into either or both lines in accordance with its prescription-program. Turning now to FIG. 22E, capable of healing, a vascular, intestinal, ureteral, or endocrine duct autograft, allograft, or successfully tissue engineered replacement segment would be considerably facilitated in this regard through the continuously coordinated control of flow-through and medicinal release through either of the two routes. Any such replacement segment could benefit from the application of antimicrobial, anti-inflammatory, and possibly angiogenesis-promotive drugs.

### Means for Promoting the Healing of Anastomoses

Drugs and nondrug materials such as stem cells, vascular epithelial growth factor signal protein, and apparently platelet rich plasma, have been identified as promoting the healing of intestinal anastomoses. The healing of mucosal anastomoses is spontaneously implemented by platelet-derived growth factor and transforming growth factor-β as well as vascular endothelial growth factor (see, for example, Morgan, R. B. and Shogan, B. D. 2022. “The Science of Anastomotic Healing,” Online, Seminars in Colon and Rectal Surgery (Philadelphia, Pennsylvania) 33(2): 100879; (see, also, for example, Geropoulos, G., Psarras, K., Papaioannou, M., Geropoulos. V., Niti, A., and 7 others 2024. “The Effectiveness of Adipose Tissue-derived Mesenchymal Stem Cells Mixed with Platelet-rich Plasma in the Healing of Inflammatory Bowel Anastomoses: A Pre-clinical Study in Rats, Online, MDPI [Multidisciplinary Digital Publishing Institute] Journal of Personalized Medicine (Basel, Switzerland) 14(1): 121; Shin, H. H., Park, J., Kim, Y. J., Kim, D., Jin, E. J., and Ryu, J. H. 2024. “Hydrophilic/Hydrophobic Janus Nanofibers Containing Compound K for Cartilage Regeneration,” Online, International Journal of Nanomedicine (Auckland, New Zealand) 1:19:1683-1697; Shin, H. H. and Ryu, J. H. 2023. “Bio-inspired Self-healing, Shear-thinning, and Adhesive Gallic Acid-conjugated Chitosan/Carbon Black Composite Hydrogels as Suture Support Materials,” Online, MDPI [Multidisciplinary Digital Publishing Institute] Biomimetics (Basel, Switzerland) 8(7): 542; Xiao, F., Zhu, C., Wei, X., Chen, G., and Xu X. 2023. “Shenhuang Plaster Enhances Intestinal Anastomotic Healing in Rabbits through Activation of the TGF-β [transforming growth factor beta] and Hippo/YAP [Salvador-Warts-Hippo pathway/yes-associated protein] Signaling Pathways,” Journal of Applied Biomedicine (Wroclaw, Poland) 21(4): 208-217; Yu, W., Zhou, H., Feng. X., Liang, X., Wei D, and 5 others 2023. “Mesenchymal Stem Cell Secretome-loaded Fibrin Glue Improves the Healing of Intestinal Anastomosis,” Online, Frontiers in Bioengineering and Biotechnology (Lausanne, Switzerland) 11:1103709; Trebol, J., Georgiev-Hristov, T., Pascual-Miguelanez, I., Guadalajara, H., Garcia-Arranz, M., and Garcia-Olmo, D. 2022. “Stem Cell Therapy Applied for Digestive Anastomosis: Current State and Future Perspectives,” Online, World Journal of Stem Cells (Pleasanton, California) 14(1): 117-141; Geropoulos, G., Psarras, K., Giannis, D., Martzivanou, E. C., Papaioannou, M., and 5 others 2021. “Platelet Rich Plasma Effectiveness in Bowel Anastomoses: A Systematic Review,” Online, World Journal of Gastrointestinal Surgery (Pleasanton, California) 13(12): 1736-1753; Buk, O. F., Ocak, S., Genc. B., Avcι, B., and Uzuner, H. O. 2020. “Is [sic Does] Platelet-rich Plasma Improves [sic Improve] the Anastomotic Healing in Hyperthermic Intraperitoneal Chemotherapy with Oxaliplatin: An Experimental Rat Study,” Online, Annals of Surgical Treatment and Research (Seoul, South Korea) 98(2): 89-95; Ruiz-Luque, V., Parra-Membrives, P., Escudero-Severin, C., and Aguilar-Luque, J. 2019. “Effect of Alprostadil on Colorectal Anastomoses under Relative Ischemia,” Journal of Surgical Research (New York, New York) 236:230-237; Oines, M. N., Krarup, P. M., Jorgensen, L. N., and Agren. M. S. 2014. “Pharmacological Interventions for Improved Colonic Anastomotic Healing: A Meta-analysis,” Online, World Journal of Gastroenterology (Pleasanton, California) 20(35): 12637-12648).

Some methods such as the use of a laser for promoting the healing of vascular anastomoses require direct access (see, for example, Moritz, W. R., Raman, S., Pessin, S., Martin, C., Li, X., Westman, A., and Sacks, J. M. 2022. “The History and Innovations of Blood Vessel Anastomosis,” Online, MDPI [Multidisciplinary Digital Publishing Institute] Bioengineering (Basel, Switzerland) 9(2): 75; Pabittei. D. R., de Boon, W., Heger, M., van Golen, R. F., Balm, R., Legemate, D. A., and de Mol. B. A. 2015. “Laser-assisted Vessel Welding: State of the Art and Future Outlook,” Online, Journal of Clinical and Translational Research (Singapore) 1(2): 1-18; Burger, R. A., Gerharz, C. D., Rothe, H., Engelmann, U. H., and Hohenfellner, R. 1991. “CO₂ and Nd:YAG [neodymium-doped yttrium aluminum gamet;] Laser Systems in Microsurgical Venous Anastomoses,” Urological Research (Berlin, Germany) 19(4): 253-237; Mccarthy, W. J., Hartz, R. S., Yao. J. S., Sottiurai. V. S., Kwaan, H. C., and Michaelis, L. L. 1986. “Vascular Anastomoses with Laser Energy,” Online, Journal of Vascular Surgery (New York, New York) 3(1): 32-41).

However, research has also identified drugs and nondrug agents which are also beneficial in this regard (see, for example, Seipelt, R. G., Backer, C. L., Mavroudis, C., Stellmach, V., Seipelt. I. M., and 3 others 2003. “Topical VEGF [vascular epithelial growth factor] Enhances Healing of Thoracic Aortic Anastomosis for Coarctation in a Rabbit Model,” Online, Circulation (Hagerstown. Maryland) 108 Supplement 1:11150-11154; shortly above in this section).

### Placing a DC Resting Potential Across the Opposing Edges of the Anastomosis

Another long-established means for accelerating the healing of a wound is to place a DC resting potential across the severed edges of the stumps, something an implanted automatic disorder response system can easily provide in coordination with the direct targeting of medication to the site. Briefly, the free ends of the stumps are encircled one by an annular anode and the other by a cathode attached to a source of current such as a transcutaneously rechargeable button cell. These components for promoting healing in vascular, neural, intestinal, or glandular duct anastomoses, especially when refractory to prompt healing or risking a leak from the gut if not well mended, can be used by the implanted disorder response system. Anastomosis and wound healing by first intention is not limited to cylindrical structures which have been transected but pertains to the apposition and suturing together of any kind of tissue along a common line.

Pertinent to solid organ transplantation generally, nerve regeneration and the recovery of nervous control is also addressed above in relation to heart transplantation, the vagus nerve, and composite tissue transplantation to include the face and eyes. These are addressed above in sections 2b(1)(a), entitled Sudden Compound Vascular Bypass, or Sudden Switched-organ, Transplantation, and 2b(1)(b)(b12), entitled Metered Compound Vascular Bypass, or Switched-organ, Vascularized Composite Tissue Allotransplantation as well as in other sections. Anastomosis and wound healing by first intention is not limited to cylindrical structures but pertains to the apposition and suturing together of tissues along a common line.

Made sufficiently lightweight and small in relation to the substrate structure and recharged by means of transcutaneous energy transfer, the need for retrieval requiring reentry after healing can be eliminated (see, for example, Kong, J., Teng. C., Liu, F., Wang. X., Zhou, Y., and 6 others 2024. “Enhancing Regeneration and Repair of long-distance Peripheral Nerve Defect Injuries with Continuous Microcurrent Electrical Nerve Stimulation,” Online, Frontiers in Neuroscience (Lausanne, Switzerland) 18:1361590; Narayanan, R. P., Khaleghi, A., Veletic. M., and Balasingham, I. 2024. “Multiphysics Simulation of Magnetoelectric Micro Core-shells for Wireless Cellular Stimulation Therapy via Magnetic Temporal Interference,” Online, PLoS [Public Library of Science] One (San Francisco, California) 19(1): e0297114; Rabbani, M., Rahman, E., Powner, M. B., and Triantis, I. F. 2024. “Making Sense of Electrical Stimulation: A Meta-analysis for Wound Healing,” Online, Annals of Biomedical Engineering (New York, New York) 52(2): 153-177; Zou, X., Dong, Y., Alhaskawi, A, Zhou, H., Ezzi, S. H. A., and 5 others 2024. “Techniques and Graft Materials for Repairing Peripheral Nerve Defects,” Online, Frontiers in Neurology (Lausanne, Switzerland) 14:1307883; Juckett. L., Saffari, T. M., Ormseth, B., Senger, J. L., and Moore, A. M. 2022. “The Effect of Electrical Stimulation on Nerve Regeneration following Peripheral Nerve Injury,” Online, MDPI [Multidisciplinary Digital Publishing Institute] Biomolecules (Basel, Switzerland) 12(12): 1856; Patel, S., Ershad, F., Zhao, M., Isseroff, R. R., Duan. B/, and 3 others 2022. “Wearable Electronics for Skin Wound Monitoring and Healing,” Online, Soft Science (Alhambra, California) 2022:2:9; Verdes, M., Mace, K., Margetts. L., and Cartmell, S. 2022. “Status and Challenges of Electrical Stimulation Use in Chronic Wound Healing,” Current Opinion in Biotechnology (London, England) 75:102710; Rajendran, S. B., Challen, K., Wright, K. L., and Hardy, J. G. 2021. “Electrical Stimulation to Enhance Wound Healing,” Online, MDPI [Multidisciplinary Digital Publishing Institute] Journal of Functional Biomaterials (Basel, Switzerland) 12(2): 40; Thakral, G., Lafontaine, J., Najafi. B., Talal. T. K., Kim, P., and Lavery, L. A. 2013. “Electrical Stimulation to Accelerate Wound healing,” Online, Diabetic Foot and Ankle (Philadelphia, Pennsylvania) September 16:4; Kennelly, R., Conneely, J. B., Bouchier-Hayes, D. J., and Winter, D. C. 2011. “Electrical Field Stimulation Promotes Anastomotic Healing in Poorly Perfused Rat Colon,” International Journal of Colorectal Disease (New York, New York) 26(3): 339-44; Balakatounis, K. C. and Angoules, A. G. 2008. “Low-intensity Electrical Stimulation in Wound Healing: Review of the Efficacy of Externally Applied Currents Resembling the Current of Injury,” Online, Eplasty (Malvern, Pennsylvania) 8: e28).

Of potential importance in transplantation, electrical means, along with immunomodulatory and conductive hydrogels, can also promote healing (see, for example, Hu, Y. W., Wang, Y. H., Yang, F., Liu, D. X., Lu, G. H., and 16 others 2024. “Flexible Organic Photovoltaic-powered Hydrogel Bioelectronic Dressing with Biomimetic Electrical Stimulation for Healing Infected Diabetic Wounds,” Online, Advanced Science (Weinheim, Baden-Württemberg, Germany) 11(10): e2307746; Yang, P., Lu, Y., Gou, W., Qin, Y., Tan, J., Luo. G., and Zhang, Q. 2024. “Glycosaminoglycans' Ability to Promote Wound Healing: From Native Living Macromolecules to Artificial Biomaterials,” Online, Advanced Science (Weinheim, Baden-Württemberg, Germany) 11(9): e2305918; Roy Barman, S. and Jhunjhunwala, S. 2023. “Electrical Stimulation for Immunomodulation,” Online, ACS [American Chemical Society] Omega (Washington, D. C.) 9(1): 52-66; Cao, J., Wu, B., Yuan, P., Liu. Y., and Hu. C. 2023. “Rational Design of Multifunctional Hydrogels for Wound Repair,” Online, MDPI [Multidisciplinary Digital Publishing Institute] Journal of Functional Biomaterials (Basel, Switzerland) 14(11): 553; Tan, W., Long. T., Wan, Y., Li, B., Xu. Z., and 4 others 2023. “Dual-drug Loaded Polysaccharide-based Self-healing Hydrogels with Multifunctionality for Promoting Diabetic Wound Healing,” Carbohydrate Polymers (Barking, East London, England) 312:120824; Asadi, N., Pazoki-Toroudi, H., Del Bakhshayesh, A. R., Akbarzadeh, A., Davaran, S., and Annabi, N. 2021. “Multifunctional Hydrogels for Wound Healing: Special Focus on Biomacromolecular Based Hydrogels,” International Journal of Biological Macromolecules (Amsterdam, North Holland, Netherlands) 170:728-750; Cheah, Y. J., Buyong, M. R., and Mohd Yunus, M. H. 2021. “Wound Healing with Electrical Stimulation Technologies: A Review,” Online, MDPI [Multidisciplinary Digital Publishing Institute] Polymers (Basel, Switzerland) 13(21): 3790; Kharaziha, M., Baidya, A., and Annabi, N. 2021. “Rational Design of Immunomodulatory Hydrogels for Chronic Wound Healing,” Advanced Materials (Deerfield Beach, Florida/Weinheim an der Bergstrasse, Germany) 33(39): e2100176; Lu, Y., Wang. Y., Zhang, J., Hu. X., Yang, Z., Guo, Y., and Wang, Y. 2019. “In-situ Doping of a Conductive Hydrogel with Low Protein Absorption and Bacterial Adhesion for Electrical Stimulation of Chronic Wounds,” Acta Biomaterialia (Kidington, Oxford, England) 89:217-226ann).

Allografts and allograft anastomoses would additionally benefit from the ability of an implanted automatic disorder response system, usually the same which had administered the transplant, to directly pipe-target medication to include that anti-rejection to the affected site or sites thereby precluding any significant exposure to nontargeted tissue to minimize if not eliminate adverse side effects. Anti-rejection immunosuppressants can be prescribed for automatic release, while medication to treat less predictable sequelae can be released on the basis of sensor inputs.

Sensors are addressed above in section 2a, entitled Background of the Invention. General. U.S. Pat. No. 11,389,171, entitled Integrated System for the Infixion and Retrieval of Implants, and U.S. Pat. No. 11,759,186, entitled Ductus Side-entry Jackets and Prosthetic Disorder Response Systems describe means for preventing the continued passage of such medication into the systemic or pulmonary circulation and nonaffected tissue.

“The ideal immunosuppressive drug has four features: it is capable of selectivity, synergy, and specificity, and it can overcome sensitization of the recipient to the transplant,” (Institute of Medicine (US) Committee on Xenograft Transplantation: Ethical Issues and Public Policy. Xenotransplantation: Science, Ethics, and Public Policy. Washington (DC): National Academies Press (US): 1996. Part C. Immunosuppression in Allotransplantation)(see also, for example, Szumilas, K., Wilk, A., Wiśniewski, P., Gimpel, A., Dziedziejko, V., Kipp, and M., Pawlik, A. 2023. “Current Status Regarding Immunosuppressive Treatment in Patients after Renal Transplantation, Online, MDPI [Multidisciplinary Digital Publishing Institute] International Journal of Molecular Sciences (Basel, Switzerland) 24(12): 10301; Callemeyn, J., Lamarthee. B., Koenig, A., Koshy, P., Thaunat. O., and Naesens. M. 2022. “Allorecognition and the Spectrum of Kidney Transplant Rejection,” Online, Kidney International (New York, New York) 101(4): 692-710; Duneton, C., Winterberg, P. D., and Ford, M. L. 2022. “Activation and Regulation of Alloreactive T Cell Immunity in Solid Organ Transplantation,” Online, Nature Reviews. Nephrology (London, England) 18(10): 663-676; Hussain, Y. and Khan, H. 2022. “Immunosuppressive Drugs,” Online, Encyclopedia of Infection and Immunity (Amsterdam, North Holland, Netherlands) 2022:726-740; Parlakpinar, H. and Gunata, M. 2021. “Transplantation and Immunosuppression: A Review of Novel Transplant-related Immunosuppressant Drugs,” Immunopharmacology and Immunotoxicology (London, England) 43(6): 651-665; Viklicky, O., Slatinska, J., Novotny, M., and Hruba, P. 2021. “Developments in Immunosuppression,” Current Opinion in Organ Transplantation (Hagerstown, Maryland) 26(1): 91-96).

Direct pipe-targeting makes possible not only the focusing of immunosuppressants on the allograft and its anastomoses, but the interception of its dendritic leukocytes en route to the host spleen and lymph nodes through their respective blood, not lymph, supplies. Sensors to detect signs of rejection are addressed above in section 2a, entitled Background of the Invention. General, in the subsection entitled Sensors, paragraph e. Graft organ rejection analytes and immunosensors.

Before 1990, it was becoming clear that the methods and routes used by donor alloantigens are neither few nor simple, making the ability to literally, that is, mechanically, target rather than systemically disperse drugs highly advantageous both in minimizing side effects while at the same time allowing the use of higher doses (Larsen, C. P., Morris, P. J., and Austyn, J. M. 1990. “Migration of Dendritic Leukocytes from Cardiac Allografts into Host Spleens,” A Novel Pathway Initiation of Rejection,” Online, Journal of Experimental Medicine (New York, New York) 171(1): 307-314)(see also, Balint, M. T., Lemajić, N., Jurišić, V., Pantelić, S., Stanisavljević, D., Kurtović, N. K., and Balint, B. 2024. “An Evidence-based and Risk-adapted GSF [granulocyte stimulating factor] versus GSF Plus Plerixafor Mobilization Strategy to Obtain a Sufficient CD34+ Cell Yield in the Harvest for Autologous Stem Cell Transplants,” Online, Translational Oncology (Ann Arbor, Michigan) 39:101811; Lonez, C. and Breman, E. 2024. “Allogeneic CAR-T [chimeric antigen receptor T-cell] Therapy Technologies: Has the Promise Been Met?,” Online, MDPI [Multidisciplinary Digital Publishing Institute] Cells (Basel, Switzerland) 13(2): 146; Camel, N., Lancia, H. H., Guinier, C., and Benichou, G. 2023. “Pathways of Antigen Recognition by T Cells in Allograft Rejection,” Online, Transplantation (Hagerstown, Maryland) 107(4): 827-837; Siu, J. H. Y., Surendrakumar, V., Richards, J. A., and Pettigrew, G. J. 2018. “T Cell Allorecognition Pathways in Solid Organ Transplantation,” Online, Frontiers in Immunology (Lausanne, Switzerland) 9:2548; Ali, J. M., Bolton, E. M., Bradley, J. A., and Pettigrew, G. J. 2013. “Allorecognition Pathways in Transplant Rejection and Tolerance, Online, Transplantation (Hagerstown, Maryland) 96(8): 681-688; Game, D. S. and Lechler, R. I. 2002. “Pathways of Allorecognition: Implications for Transplantation Tolerance,” Transplantation Immunology (Amsterdam, North Holland, Netherlands) 10(2-3): 101-108; Harris, P. E., Cortesini. R., and Suciu-Foca, N. 1999. “Indirect Allorecognition in Solid Organ Transplantation,” Reviews in Immunogenetics (Copenhagen, Denmark) 1(3): 297-308).

Once an anastomosis bypass is created toward the end of a compound vascular bypass organ transplant, a lower passive flow director not requiring control ability can often be used. This is explained above in section 2b, entitled Vascular Valves and Servovalves in the Diversion of Blood Flow, in the subsection thereof entitled Simple Flow Diversion Side-entry Jackets, of which one type each is shown in FIGS. 10F and 10G. Replacement without interruption in blood flow is by local bypass of the operative site using an upstream solenoid valve, tube cut to length, and a downstream diversion jacket as specified above. Bypasses for autologous repairs are made in different sizes and calibers of connectors and tubing to match the structure requiring repair.

The tubing is cut to size and inseparably connected from the outlet of the upper sending valve, part number 9 in FIG. 1, to the inlet of the lower receiving diversion jacket by scoring the tube-connector interface surfaces, assuring a tight friction fit, and the application of a strong adhesive. Susceptible to hydrolytic and enzymatic breakdown, adhesives can be ideal for bonding incisions and anastomoses until healed. For permanent use, however, adhesives require to be isolated from the internal milieu. Inorganic interfaces joining lengths of metal and polymeric tubing where a ferrule isolates the interface can remain bonded by an adhesive indefinitely.

Hemostasis as important as the tenacity of adhesion, the anastomosis itself can be coated with a hemostatic adhesive to assure leak-free flow-through while healing (see, for example, Du, J., Liu, X., Yarema, K. J., and Jia, X. 2022. “Glycoengineering Human Neural Stem Cells (hNSCs) for Adhesion Improvement Using a Novel Thiol-modified N-acetylmannosamine (ManNAc) Analog,” Online, Materials Science and Engineering: C. Materials for Biological Applications 134:112675; Li, K., Zan, X., Tang, C., Liu, Z., Fan, J., and 5 others 2022. “Tough, Instant, and Repeatable Adhesion of Self-healable Elastomers to Diverse Soft an Hard Surfaces,” Online, Advanced Science (Wiley Verlag Chemie, Weinheim, Germany) 9(12): 2105712; Li, Q., Song, W., Li, J., Ma, C., Zhao, X., and 5 others 2022. “Bioinspired Super-strong Aqueous Synthetic Tissue Adhesives,” Matter (Cambridge, Massachusetts) 5(3): 933-956; Lutz, T. M., Kimna, C., Casini, A. and Lieleg, O. 2022. “Bio-based and Bio-inspired Adhesives from Animals and Plants for Biomedical Applications,” Online, Materials Today Bio 13:100203; Yin, P., Liu Y., Huang, D., and Zhang, C. 2022. “Highly Tough, Stretchable, and Self-healing Polyampholyte Elastomers with Dual Adhesiveness,” Online, MDPI [Multidisciplinary Digital Publishing Institute] International Journal of Molecular Sciences (Basel, Switzerland) 23(9): 4548; Zou, C.-Y., Lei, X. X., Hu, J.-J., Jiang, Y.-L., Li, Q.-J., and 4 others 2022. “Multi-crosslinking Hydrogels with Robust Bio-adhesion and Pro- coagulant Activity for fort-aid Hemostasis and Infected Wound Healing,” Online, Bioactive Materials 16:388-402; Bal-Ozturk, A., Cecen, B., Avci-Adali, M., Topkaya, S. N., Alarcin, E., and 8 others 2021. “Tissue Adhesives: From Research to Clinical Translation,” Online, Nano Today 36:101049; Ma, C., Sun, J., Li, B., Feng, Y., Sun, Y., and 19 others 2021. “Ultra-strong Bio-glue from Genetically Engineered Polypeptides,” Online, Nature Communications (London, England) 12(1): 3613; Nam, S. and Mooney, D. 2021. “Polymeric Tissue Adhesives,” [American Chemical Society] Chemical Reviews 121(18): 11336-11384; Park, J., Kim, Y., Chun, B., and Seo, J. 2021. “Rational Engineering and Applications of Functional Bioadhesives in Biomedical Engineering,” Biotechnology Journal 16(12): e2100231; Bao, Z., Gao, M., Sun, Y., Nian, R., and Xian, M. 2020. “The Recent Progress of Tissue Adhesives in Design Strategies, Adhesive Mechanism, and Applications,” Online, Materials Science and Engineering: C. Materials for Biological Applications 111:110796; Ge, L. and Chen, S. 2020. “Recent Advances in Tissue Adhesives for Clinical Medicine,” Online, Polymers (Basel, Switzerland) 12(4): 939; Burks, S. and Spotnitz, W. 2014. “Safety and Usability of Hemostats, Sealants, and Adhesives,” AORN [Association of Operative Registered Nurses] Journal 100(2): 160-176; Sanders, L. and Nagatomi, J. 2014. “Clinical Applications of Surgical Adhesives and Sealants,” Online, Critical Reviews in Biomedical Engineering 42(3-4): 271-292; Gabay, M. and Boucher, B. A. 2013. “An Essential Primer for Understanding the Role of Topical Hemostats, Surgical Sealants, and Adhesives for Maintaining Hemostasis,” Pharmacotherapy 33(9): 935-955; Duarte, A. P., Coelho, J. F., Bordado, J. C., Cidade, M. T., and Gil, M. H., 2012. “Surgical Adhesives: Systematic Review of the Main Types and Development Forecast,” Online, Progress in Polymer Science 37(8): 1031-1050).

FIG. 22C shows the application of a bypass in autologous anastomosis, where a segment along a vessel or nerve was accidently or surgically transected or a segment thereof was irreparably crushed and/or had to be surgically removed. In FIG. 22C, a vascular, neural, gamete delivery, gland duct, ureter, or the gut has been transected and anastomosed, where the ability to temporarily divert flow through a bypass in order to allow the directly targeted application of an antimicrobial or anti-inflammatory, for example, to the anastomosis would afford protection and expedite healing.

In FIGS. 7 and 8, is a plunger solenoid—part number 32—driven flow diversion valve with a drug delivery line, (drugline, accessory channel) that with its diversion chute completely extended to divert flow as shown in FIG. 2, for example, allows the release medication to flow down over the anastomosis while bypassed through drugline 8′, while at the same time allowing the release of medication through drugline, or accessory channel 8 into the bypass flow. The upper valve is provided with a length of tubing securely bonded at its underside and can be cut to length. In contrast to FIG. 22C wherein direct conjunction and anastomosis of the stump ends is possible, FIG. 22D depicts the situation where an intervening segment of a vessel, nerve, duct, or the gut, whether having been crushed, avulsed, or surgically resected, has been lost.

Except for peristaltic ductus such as an oviduct or the gut or the colon more so where the gap will only temporarily delay recovery of nervous conduction along the motility-driving plexuses, hence, of peristalsis, with inflammation and impaired permeability, stumps such as vascular can be connected by side-entry diversion jackets and a length of tubing (see, for example, Clatterbuck, B and Moore, L. 2023. “Small Bowel Resection,” Online, Treasure Island, Florida: StatPearls Publishing Co.; Fujimoto T, Manabe T, Yukimoto K, Tsuru Y, Kitagawa H, and 4 others 2023. “Risk factors for Postoperative Paralytic Ileus in Advanced-age Patients after Laparoscopic Colorectal Surgery: A Retrospective Study of 124 Consecutive Patients,” Online, Journal of the Amis, Rechon and Colon (Tokyo, Japan) 7(1): 30-37; Khawaja, Z. H., Gendia, A., Adnan, N., and Ahmed, J. 2022. “Prevention and Management of Postoperative Ileus: A Review of Current Practice,” Online, Cureus (Palo Alto, California) 14(2): e22652; Lin, Z., Li, Y., Wu, J., Zheng, H., and Yang, C. 2022. “Nomogram for Prediction of Prolonged Postoperative Ileus after Colorectal Resection,” BioMed Central Cancer (London, England) 22(1): 1273; Kim, Y. M., Hussain, Z., Lee, Y. J., and Park, H. 2021. “Altered Intestinal Permeability and Drug Repositioning in a Post-operative Ileus Guinea Pig Model,” Online, Journal of Neurogastroenterology and Motility 27(4): 639-649; Lubawski, J. and Saclarides, T. 2008. “Postoperative Ileus: Strategies for Reduction, Online, Therapeutics and Clinical Risk Management 4(5): 913-917).

This situation, pertinent to a compound vascular bypass double heart transplant, for example, would best be ameliorated through the interpositioning of a synthetic segment consisting of a length of plastic tubing, to fill the gap, thus both reinstating continuity of flow through, preventing the stumps from atrophy, while at the same time allowing the placement of a bypass with flow switching. In an infant, to considerably defer if not eliminate the need for reentry following growth, this length of tubing as any other is made of the expandable fabric described above in section 2b(2)(f), entitled Expandable Structure to Accommodate Growth and Self-adjust in Size in Vascular Segment Prostheses, Lines, and Confluence Conduits Placed in Children, as shown in FIGS. 15C, 15D, 36A, and 36F.

FIG. 22D shows the interpositioning of a length of synthetic prosthetic tubing in a nonperistaltic ductus where the resumption in flow is essential but due to surgical resection or avulsion as the result of a traumatic injury, the stump ends cannot be brought together. The expectation with such an interpositioning is that the upstream propulsive action will push the luminal contents through the duct to the level where peristalsis is resumed, for which purpose the length of the synthetic tube must be limited.

Except in a patient at the end of life, such a repair would be a temporary emergency expedient pending proper repair with the interpositioning of an allograft or tissue engineered tissue. The interposed segment can be fixed in place as shown in FIG. 31C. Such a repair is unsuited to a peristaltic ductus where the interpositioning of a length of synthetic tubing would interrupt the nervous plexuses that propagate the propulsive waves.

With reference to FIG. 22E, given that the gut in an adult is usually over 20 feet in length, except as the result of a catastrophic injury, direct end-to-end anastomosis will rarely prove impossible. Accordingly, the need for an interposed section of a harvested autologous or allogeneic peristaltic ductus or one tissue engineered is with the risk of a loss in motility arises with smaller peristaltic ductus such as a bile duct, fallopian tube, or vas deferens when irreversibly stenosed or obstructed.

Unlike the continued flow of blood through a vessel or urine through a ureter, where repair thus without the direct targeting of drugs would work at least on a temporary emergency basis, and differing from volvulus, intussusception, or paralytic ileus, where peristalsis is physically obstructed but the plexes unsevered, the condition shown in FIG. 22D is strictly surgical, and could never appear in nature. Accordingly, the likelihood that a spontaneous recovery mode would obtain is contrary to expectations.

Crohn's disease produces a significant reduction, sometimes even an interruption in peristalsis; however, the gut plexuses have not been physically transected, or divided (Guglielmo, F. F., Mitchell, D. G., O'Kane, P. L., Deshmukh, S. P., Roth, C. G., and 4 others 2015. “Identifying Decreased Peristalsis of Abnormal Small Bowel Segments in Crohn's Disease Using Cine MR [magnetic resonance] Enterography: The Frozen Bowel Sign,” Abdominal Imaging 40(5): 1150-1156).

Given the nonoccurrence of the condition in nature, a resumption in peristalsis past a plastic tube filled gap in the severed lower distal portion of the gut attributable to collateral innervation of some alternative mechanism for communication through which the severed segment might be mobilized remains unresolved despite what appears from a speculative standpoint to represent potential evidence to the contrary (see, for example, Shikaya, Y., Takase, Y., Tadokoro, R., Nakamura, R., Inaba, M., and Takahashi, Y. 2022. “Distribution Map of Peristaltic Waves in the Chicken Embryonic Gut Reveals Importance of Enteric Nervous System and Inter-region Cross Talks along the Gut Axis,” Online, Frontiers in Cell and Developmental Biology 10:827079; Layunta, E., Buey, B., Mesonero, J. E., and Latorre, E., 2021. “Crosstalk between Intestinal Serotonergic System and Pattern Recognition Receptors on the Microbiota-Gut-Brain Axis,” Online, Frontiers in Endocrinology 12:748254; Sun, L. J., Li, J. N., and Nie, Y. Z., 2020. “Gut Hormones in Microbiota-gut-brain Cross-talk,” Online, Chinese Medical Journal (English Edition) 13(7): 826-83).

Whereas the scheme depicted in FIG. 22C is universally encountered and applicable, that shown in FIG. 22D with synthetic gap fill is of infrequent and then usually temporary application. As shown in FIG. 31C, the junctions in FIG. 22D are prevented from disengagement by hooks. Compared to FIG. 22D, where a synthetic segment could not support either missing nerve other than on an untenably long-term basis or a peristaltic ductus, the situation depicted in FIG. 22E, which can support nerve and gut, shows the replacement of a missing gap in a vessel, nerve, or peristaltic duct with a like allogeneic segment connected at either end by anastomosis to the native structure.

In FIG. 22E, the anastomoses at both the upstream and downstream ends of the graft segment can be supported through the switchable flow scheme and release of medication through the upstream valve alone, or as common, or as specific, to either anastomosis, if necessary, to protect and expedite the healing of both. To avoid the need for a preliminary operation to harvest the replacement segment, such grafts are best harvested as are solid organs in compound vascular bypass transplantation from a deceased donor who had been placed on life support prior to death and maintained thereafter for use as a source of grafts.

Here the graft is not transplanted as would a solid organ using the compound vascular bypass technique because there is not the blood supply and drainage vessels that would allow those of the donor and recipient to be connected. Insertion into the gap of the recipient can, however, be accomplished quickly enough following harvesting that reperfusion injury can be reduced to the absolute minimum. In metered compound vascular bypass, or sudden switched-organ, vascularized composite tissue allotransplantation as addressed above in section 2b(1)(b)(b12) of like title, this same approach applies to composite tissue grafts where vessels to connect between donor and recipient must be anastomosed with the connectors removed or articulable vessels are lacking.

2c. Ductus Side-Entry Jackets and Vascular Valves in Urinary Diversion

A means for assuring the hygienic voiding of urine despite the lack of a lower urinary tract, intractable frequency, incontinence, excessive retention, or any other cause, or where pathology is not a factor but urination must be made deferable despite urge sensation as needed by those appearing before the public, without the risk of lower tract infection associated with Foley, male condom type, and catheters generally, constitutes a fundamental improvement in the quality of life. The prosthetic embodiment shown in FIG. 28 should make it possible to avoid numerous procedures involving much dissection and trauma performed for reasons other than oncological, several specified below in this section.

In many cases, the need for urinary diversion using autologous tissue will be avoided along with the additional complications risked due to the need for the preliminary harvesting as in bladder replacement, bladder reconstruction, detrusor myectomy myotomy, augmentation cystoplasty, and rectus abdominis detrusor myoplasty. Similarly avoidable is likely to be the need to implant an electrostimulatory neuromodulator.

Inappropriate or uncontrolled voiding associated with dementia or cognitive impairment will add the problem of drug regimen noncompliance, making the automatic release of medication by a fully implanted disorder control system directed to one or more comorbidities, combined with sanitary discharge, of benefit to those entrusted with care of the very young, the elderly, or the deranged affected thus. While the lower urinary tract prosthesis shown in FIG. 28 functions in the absence of urge sensation, when switched by the user to nonautomatic function, the lower urinary tract assist device shown in FIG. 30 makes it possible for the wearer with retained urge sensation and intractable frequency, for example, to void without detection while seated at a dinner table with others.

FIG. 28 shows a lower urinary tract prosthesis for a patient in whom the native tract or a portion thereof is lacking such as following surgical resection. FIG. 30 shows a urinary assist embodiment of the invention for a patient with an intractable disorder which is a constant hindrance, such as incontinence or an overactive bladder, or who must be able to defer voiding until a proper receptacle is available. By bypassing the lower tract, hence, urge sensation, virtually every disorder pertaining to voiding that impairs the ability to function in society is eliminated. The synthetic materials of which the embodiment shown in FIGS. 28 and 30 cannot become infected, hypoxic, or otherwise diseased, cannot generate pain, and can be sterilized with solutions of a potency that would rule out contact with urothelium.

In a stone former, the accretion of crystal or the formation of stones is prevented by flushing the lines through with a crystal solvent for the type of stone. An implanted control system can automatically dispense support solutions through druglines 8 and 8′ in the drawing figures. Usually associated with immaturity or dementia, for example, inappropriate voiding accompanied by drug regimen noncompliance will make the automatic release of medication, ameliorative or directed to one or more comorbidities, combined with sanitary discharge, of benefit to those entrusted with care of such patients.

The automatic administration of medication, antiseptics, and crystal solvents is entrusted to a fully implanted disorder response system as described in U.S. Pat. No. 11,759,186, entitled Ductus Side-entry Jackets and Prosthetic Disorder Response Systems, and in copending application Ser. No. 17/689,880, entitled Prosthetic Disorder Response Systems. Such a system can directly pipe-target medication to the specific site where it is needed through a drugline inserted into an accessory channel of the side-entry device whether an adjustable blood, urine, or drug flow diversion jacket, connector, valve, or servovalve at the site. Side-entry devices are described in U.S. Pat. No. 11,759,186 as well as in this specification.

Bypassing the lower urinary tract, the universally prevalent problems of urinary retention in men due most often to benign prostate hyperplasia, bladder post void residual volume due to laxity of the detrusor with age, refractory male or female urinary retention intrinsic or induced by certain drugs such as trospium chloride, urinary incontinence, urge sensation too intense and/or the need to void in public without adequate time to access a proper receptacle, and adult nocturnal enuresis, or combined urinary incontinence and bed wetting, are all overcome. In contrast, the urinary prosthesis shown in FIG. 28 is intended to replace the need for surgical reconstructions using intestinal tissue susceptible to a gradual metaplastic transition to carcinoma and susceptible to infection where the lower tract is missing, irreparably damaged, or otherwise dysfunctional.

Bypassing the lower urinary tract also eliminates the need for drugs that are of negligible effectiveness and carry numerous potentially serious complications. Botulinum toxin can be effective for up to nine months when a repeat injection is needed, but it too has numerous serious side effects. In contrast, a urinary assist device such as shown in FIG. 30 calls for surgical placement but completely eliminates the need for drugs. For this reason, the problems encountered with drugs have been enumerated to clarify the value in a device that eliminates their need.

The lower urinary tract bypass device, or urinary assistance drainage system shown in FIG. 30 is not a prosthesis used to replace native tissue, although the size of the system neobladder part number 145 in FIG. 30 will be limited to accommodate the native bladder. Rather it can serve the need of anyone for any reason as a voluntarily activated assist or catch mechanism when the situation of the patient is inopportune for access to a bathroom. Such needs include nocturia wherewith intense urge arises during sleep or otherwise during the course of a public performance (see, for example, Chestnut, G. T. Rentea, R. M., and Leslie, S. W. 2023. “Urinary Diversions and Neobladders,” Online, Treasure Island, Florida: StatPearls Publishing Company; Leslie; S. W., Rawla, P., and Dougherty, J. M. 2023. “Female Urinary Retention,” Online, Treasure Island, Florida: StatPearls Publishing Co.; Leslie; S. W., Sajjad, H., and Singh, S. 2023. “Nocturia,” Online, Treasure Island, Florida: StatPearls Publishing Co.; Ballstaedt; L and Woodbury, B. 2022. “Bladder Post Void Residual Volume,” Online, Treasure Island, Florida: StatPearls Publishing Co.; Dougherty, J. M. and Aeddula, N. R. 2022. “Male Urinary Retention,” Online, Treasure Island, Florida: StatPearls Publishing Co.; Song, Q. X., Li, J., Gu, Y., Xu. L., Abrams, P., and Xue, W. 2022. “The Clinical Features and Predictive Factors of Nocturnal Enuresis in Adult Women,” Online, Frontiers in Medicine (Lausanne, Switzerland). 8:744214; Song, Q. X., Wang, L., Cheng, X., Hao, Y., Liu Z., and Abrams, P. 2020. “The Clinical Features and Predictive Factors of Nocturnal Enuresis in Adult Men,” British Journal of Urology International (Oxford, England) 126(4): 472-480; Akhavizadegan, H., Locke, J. A., Stothers, L., and Kavanagh, A. 2019. “A Comprehensive Review of Adult Enuresis,” Online, Canadian Urological Association Journal (Dorval, Quebec City, Canada) 13(8): 282-287).

In otherwise healthy, usually elderly, patients, urinary retention, or an inability to urinate—which is a medical emergency—or high post voiding residual volume remaining in the bladder, often with urge sensation unremitting and painful are common complaints. The collection systems shown in FIGS. 28 and 30 eliminate this problem by diverting urine directly from the ureters into a collection bag cinched about the thigh or directly into a bathroom urinal, thus bypassing the bladder floor as the seat of urge sensation centered on the trigone, or bladder outlet. These conditions if chronic may justify the prosthesis, or if episodic may warrant the embodiment of a patient-controllable assistance system shown in FIG. 30.

Takeoff at the ureters in a prosthesis such as shown in FIG. 28 is appropriate in response to agenesis, or aplasia, of the bladder, or irreparable trauma such as following a radical cystectomy, or an exenteration, or evisceration, of the lower, or distal, urinary tract. The use of a synthetic prosthesis eliminates the need for reconstructive surgery that poses numerous risks of complications.

This means for bladder replacement enables the resection of an otherwise intractably dysfunctional and/or painful bladder as may arise not only with malignancy but interstitial cystitis, for example (see, for example, Barone, B., Napolitano, L., Reccia, P., Calace, F. P., De Luca, L., and 23 others 2024. “Advances in Urinary Diversion: From Cutaneous Ureterostomy to Orthotopic Neobladder Reconstruction—A Comprehensive Review,” Online, MDPI [Multidisciplinary Digital Publishing Institute] Journal of Personalized Medicine (Basel, Switzerland) 14(4): 392; El-Helaly, H. A., Saifelnasr, M. K., Mohamed, K. M., Abdelaziz, A. S., and Youssof, H. A. 2019. “Outcome of Orthotopic Sigmoid versus Ileal Neobladder Reconstruction,” Online, Urology Annals (Mumbai (Bombay) India) 11(2): 204-210; Hu, S. W., Wu, C. C., Chen, K. C., and Ho, C. H. 2019. “Modified U-shaped Ileal Neobladder Designed for Facilitating Neobladder-urethral Anastomosis in Extracorporeal Reconstruction after Robotic-assisted Radical Cystectomy,” Online, Journal of Cancer Research and Therapeutics ((Mumbai (Bombay) India)) 15 (Supplement): S51-S55; Martini, A., Villari, D., and Nicita, G. 2017. “Editorial Comment to: Ileal versus Sigmoid Neobladder as Bladder Substitute after Radical Cystectomy for Bladder Cancer: A Meta-analysis,” Online, International Journal of Surgery (Philadelphia, Pennsylvania) 37:13-14; Tao, S., Long, Z., Zhang, S., Chen, S. S., Zhu, D., Shi, X. J., and Tan, W. L. 2016. “Ileal versus Sigmoid Neobladder as Bladder Substitute after Radical Cystectomy for Bladder Cancer: A Meta-analysis,” Online, International Journal of Surgery (Philadelphia, Pennsylvania) 27:39-45).

In some patients, a neobladder reconstructed with digestive tissue following an exenteration, or evisceration, bodes an inordinate susceptibility to the development of malignancy (see, for example, Bacalbaşa. N., Balescu, I., and Brasoveanu, V. 2016. “Total Pelvic Supralevator Exenteration with Ileo-Colic Orthotopic Neobladder for Locoregional Recurrence after Cervical Cancer—A Case Report,” Online, Chirurgia (Bucharest, Romania). 111(1): 80-86; De Sutter. T., Akand. M., Albersen, M., Everaerts. W., Van Cleynenbreugel. B., and 7 others 2016. “The N-shaped Orthotopic Ileal Neobladder: Functional Outcomes and Complication Rates in 119 Patients,” SpringerPlus (Cham, Switzerland) 5:646). In such cases, the placement of a prosthetic bladder may prove futile.

Not adjustable, the lower tract prosthesis shown in FIG. 28 is appropriate for placement in patients who have undergone a pelvic exenteration or evisceration as a malignancy eradication measure, or have a dysfunctionally injured lower tract due to an accident, those bom with agenesis of the lower urinary tract or a certain component thereof, and those in whom a dysfunction such as one of those just cited is not a nuisance or intermittent, but rather unremitting and incurable. If intractable to medication, urinary problems that are intermittent with no loss of urinary structures is more suited to the patient-adjustable embodiment shown in FIG. 30.

Hybrid systems that combine intact native structure where other parts of the system are malformed or missing with functionally equivalent components of the prosthetic system are not addressed as endlessly variable and rare. Combination repairs where there is a unilateral defect should be clear-cut without the need for comment. Moreover, if almost all of the native system is defective, it is better to remove the parts that are not before placing the prosthesis. When supported by an implanted disorder response system, druglines leading from one or more drug reservoirs positioned subcutaneously in the pectoral region can be used to automatically, periodically, and directly pipe-target a sanitizer or crystal solvent directly into the device.

Otherwise, the device can be flushed through by connection to its outlet tube, or neourethra, part number 110 in the drawing figures. These systems eliminate the need for a sacral electrostimulatory neuromodulator to treat urinary problems. However, such devices are beneficial for the treatment of gastrointestinal motility problems, for example, automatically by an implanted disorder response system (Jiang, Y. and Soffer, E. 2023. “Electroceuticals for Neurogastroenterology and Motility Disorders,” Online, Current Gastroenterology Reports (Philadelphia, Pennsylvania) 25(4): 91-97; Wang, X. and Chen, J. D. 2023. “Therapeutic Potential and Mechanisms of Sacral Nerve Stimulation for Gastrointestinal Diseases,” Online, Journal of Translational Internal Medicine (Warsaw, Poland) 11(2): 115-127).

In FIG. 1, which first appeared as FIG. 12A in U.S. Pat. No. 11,013,858, entitled Nonjacketing Side-entry Connectors and Prosthetic Disorder Response System, a ductus side-entry jacket placed high on the ureter beneath the ureteropelvic, or pelviureteric, junction makes it possible to pass a ureteroscope, angioscope, or intravascular ultrasound probe, for example, from a small port at the body surface down through the ureter and into the bladder, where this downward antegrade, rather than retrograde (upward), passage avoids moving through and spreading infection from the lower to the upper tract with the risk of causing urosepsis (see, for example, Kaczmarek, K., Jankowska, M., Kalembkiewicz, J., Kienitz, J., Chukwu, O., Leminski, A., and Słojewski, M. 2024. “Assessment of the Incidence and Risk Factors of Postoperative Urosepsis in Patients Undergoing Ureteroscopic Lithotripsy,” Online, Central European Journal of Urology (Warsaw, Poland) 77(1): 122-128. Wang, S., Zhao, S., Jin, S., Ye, T., and Xinling, P. 2024. “Sepsis Risk in Diabetic Patients with Urinary Tract Infection,” Online, PLoS [Public Library of Science] One (San Francisco, California) 19(5): e0303557; Bhojani, N., Miller, L. E., Bhattacharyya, S., Cutone, B., and Chew, B. H. 2021. “Risk Factors for Urosepsis after Ureteroscopy for Stone Disease: A Systematic Review with Meta-analysis,” Online, Journal of Endourology (New York, New York) 35(7): 991-1000; Scotland, K. B. and Lange, D. 2018. “Prevention and Management of Urosepsis Triggered by Ureteroscopy,” Research and Reports in Urology 10:43-49; Sohn, D. W., Kim, S. W., Hong C. G., Yoon, B. I., Ha, U. S., and Cho, Y. H. 2013. “Risk Factors of Infectious Complication after Ureteroscopic Procedures of the Upper Urinary Tract,” Journal of Infection and Chemotherapy 19(6): 1102-1108).

The decision as to whether the condition is sufficiently threatening to place the line is a clinical judgment that a more serious condition underlies a usually less severe problem than an infection of the lower urinary tract. The creation of access in reverse thus makes it possible to visually examine the urinary tract before an infection in the lower tract is eradicated, which delays diagnosis and therapy of the bladder significantly earlier than if visual examination did not have to be postponed for weeks during which an antimicrobial, usually an antibiotic, eradicated the infection. Often the first and even a second course of one or more antibiotics fails to eradicate the bacteria, extending diagnosis and treatment by another two or so weeks.

However, approach from above through this path allows clear visual examination, and if necessary, antegrade rather than more complication-prone transurethral resection or fulguration of malignant transitional urothelium (see, for example, Swanson, D. A. 2006. “Genitourinary Cancer,” in The Merck Manual, 18th edition, page 2048) immediately and thus well before the antimicrobial would eradicate the infection sometimes multibacterial and taking weeks to eradicate.

### Control of Infection

The accessory channels, or sidelines, of these side-entry devices can then be used to deliver medicinal agents directly from a body surface port to the blood supply and parenchyma of the transplant—or as a function of a prosthetic disorder response systeim—a native organ (see, for example, Klein, J., Kuperman, M., Haley, C., Barri, Y., Chandrakantan, A., Fischbach, B., Melton, L., and 5 others 2015. “Late Presentation of Adenovirus-induced Hemorrhagic Cystitis and Ureteral Obstruction in a Kidney-pancreas Transplant Recipient,” Proceedings [of the Baylor University Medical Center] 28(4): 488-491; Ersan, S., Yorukoglu, K., Sert, M., Atila, K., Celik, A., and 6 others 2012. “Unusual Case of Severe Late-onset Cytomegalovirus-induced Hemorrhagic Cystitis and Ureteritis in a Renal Transplant Patient,” Renal Failure 34(2): 247-250; Ardalan, M. 2012. “Rare Presentations of Cytomegalovirus Infection in Renal Allograft Recipients,” Nephrourology Monthly 4(2): 431-436; Thomas, M. C., Russ, G. R., Mathew, T. H., Rao Mohan, M., Cooper, J., and Walker, R. J. 2001. “Four Cases of CMV [cytomegalovirus] Ureteritis: Emergence of a New Pattern of Disease?,” Clinical Transplantation 15(5): 354-358).

A relatively common viral infection following renal or bone marrow transplantation to promote graft acceptance by chimeric means (see, for example, Cetrulo, C. L. Jr., Drijkoningen, T., and Sachs, D. H. 2015, Op cit.; Graves, S. S., Mathes, D. W., Georges, G. E., Kuhr, C. S., Chang, J., Butts, T. M., and Storb, R. 2012, Op cit.), where the need for immunosuppressant drugs increases susceptibility to infection in the urinary tract, to include inducing ureteral stenosis, for example, making the highly localized direct piping of medication, usually in higher than systemic concentration through nonjacketing side-entry connectors and jackets to levels along the urinary tract advantageous, is that caused by BK [initials of the index patient] polyomavirus as BK Virus Associated Nephropathy (BKVAN).

An effective antiviral to eradicate polyomavirus wanting, and those currently available fraught with serious risks, immunosuppressant-overriding, counteracting, or resistant agents such as hyperimmune globulins and cytomegalovirus neutralizing antibodies (see, for example, Ambalathingal, G. R., Francis, R. S., Smyth, M. J., Smith, C., and Khanna, R. 2017. “BK Polyomavirus: Clinical Aspects, Immune Regulation, and Emerging Therapies,” Clinical Microbiology Reviews 30(2): 503-528; Vigil, D., Konstantinov, N. K., Barry, M., Harford, A. M., Servilla, K. S., and 4 others 2016. “BK Nephropathy in the Native Kidneys of Patients with Organ Transplants: Clinical Spectrum of BK Infection,” Online World Journal of Transplantation (Pleasanton, California) 6(3): 472-504; Schwarz, A., Linnenweber-Held, S., Heim, A., Framke, T., Haller, H., and Schmitt, C. 2016. “Viral Origin, Clinical Course, and Renal Outcomes in Patients with BK Virus Infection after Living-Donor Renal Transplantation,” Transplantation 100(4): 844-853; Rowntree, L. C., Nguyen, T. H., Gras, S., Kotsimbos, T. C., and Mifsud, N. A. 2016. “Deciphering the Clinical Relevance of Allo-human Leukocyte Antigen Cross-reactivity in Mediating Alloimmunity Following Transplantation,” Current Opinion in Organ Transplantation 21(1): 29-39; Jeffers-Francis, L. K., Burger- Calderon, R., and Webster-Cyriaque, J. 2015. “Effect of Leflunomide, Cidofovir and Ciprofloxacin on Replication of BKPyV [nephropathy and hemorrhagic cystitis-associated BK Polyomavirus] in a Salivary Gland in Vitro Culture System,” Antiviral Research 118:46-55; Satyanarayana, G., Marty, F. M., and Tan, C. S. 2014. “The Polyomavirus Puzzle: Is Host Immune Response Beneficial in Controlling BK Virus after Adult Hematopoietic Cell Transplantation?,” Transplant Infectious Disease 16(4): 521-531; Lanzavecchia, A. and Macagno, A. 2013. Human Cytomegalovirus Neutralising Antibodies and Use Thereof, U.S. Pat. No. 8,545,848; D'Orsogna, L. J., van Besouw, N. M., van der Meer-Prins, E. M., van der Pol, P., Franke-van Dijk, M., and 8 others 2011. “Vaccine-induced Allo-HLA-reactive Memory T Cells in a Kidney Transplantation Candidate,” Transplantation 91(6): 645-651; Evers, D. L., Wang, X., Huong, S. M., Andreoni, K. A., and Huang, E. S. 2005. “Inhibition of Human Cytomegalovirus Signaling and Replication by the Immunosuppressant FK778,” Antiviral Research 65(1): 1-12; Andreoni, K. A., Wang, X., Huong, S. M., and Huang, E. S. 2002. “Human Cytomegalovirus Hyperimmune Globulin Not Only Neutralizes HCMV [human cytomegalovirus] Infectivity, But Also Inhibits HCMV-induced Intracellular NF-kappaB, Sp1, and PI3-K Signaling Pathways,” Journal of Medical Virology 67(1): 33-40.; Andreoni, K. A., Wang, X., Huong, S. M., and Huang, E. S. 2001. “Human CMV-IGIV (CytoGam) Neutralizes Human Cytomegalovirus (HCMV) Infectivity and Prevents Intracellular Signal Transduction after HCMV exposure,” Transplant Infectious Disease 3 Supplement 2:25-30) can be used to tightly localize immunosuppression or the restoration of immune function to the transplant.

Rather than forced to reduce the dose of the systemically distributed immunosuppressant drug or drugs, this allows the systemic level to be applied (see, for example, Ambalathingal, G. R., Francis, R. S., Smyth, M. J., Smith, C., and Khanna, R. 2017. “BK Polyomavirus: Clinical Aspects, Immune Regulation, and Emerging Therapies,” Clinical Microbiology Reviews 30(2): 503-528; Wunderink, H. F., van der Meijden, E., van der Blij-de Brouwer, C. S., Mallat, M. J., Haasnoot, G. W., van Zwet, E. W., Claas, E. C., and 7 others 2017. “Pretransplantation Donor-Recipient Pair Seroreactivity Against BK Polyomavirus Predicts Viremia and Nephropathy after Kidney Transplantation,” American Journal of Transplantation 17(1): 161-172; Toyoda, M., Shin, B. H., Ge, S., Mirocha, J., Thomas, D., Chu, M., Rodriguez, E., and 8 others 2017. “Impact of Desensitization on Antiviral Immunity in HLA [human leukocyte antigen]-Sensitized Kidney Transplant Recipients,” Journal of Immunology Research 2017:5672523; Pietrobon, S., Bononi, I., Mazzoni, E., Lotito, F., Manfrini, M., Puozzo, A., and 5 others 2017. “Specific IgG [immunoglobulin] Antibodies React to Mimotopes of BK Polyomavirus, a Small DNA Tumor Virus, in Healthy Adult Sera,” Online, Frontiers in Immunology (Lausanne, Switzerland) 8:236; Lo, P., Sharma, A., Craig, J. C., Wyburn, K., Lim, W., and 4 others 2016. “Preconditioning Therapy in ABO-incompatible Living Kidney Transplantation: A Systematic Review and Meta-analysis,” Transplantation 100(4): 933-942; van Aalderen, M. C., Remmerswaal, E. B., Heutinck, K. M., Ten Brinke, A., Feltkamp, M. C., van der Weerd, N. C., and 4 others 2016. “Clinically Relevant Reactivation of Polyomavirus BK (BKPyV) in HLA-A02-Positive Renal Transplant Recipients is Associated with Impaired Effector-Memory Differentiation of BKPyV-Specific CD8+ T Cells,” Online PLoS [Public Library of Science] Pathogens 12(10): e1005903; Tonnerre, P., Gerard, N., Gavlovsky P. J., Mazalrey, S., Hourmant, M., Cheneau, M. L., and 4 others 2016. “MICA [major histocompatibility complex class I-related chain A] Mutant A5.1 Influences B K Polyomavirus Reactivation and Associated Nephropathy after Kidney Transplantation,” Journal of Infectious Diseases 214(5): 807-816; Helantera, I., Hirsch, H. H., Wernli, M., Ortiz, F., Lempinen, M., and 4 others 2016. “Simultaneous BK Polyomavirus (BKPyV)-associated Nephropathy and Hemorrhagic Cystitis after Living Donor Kidney Transplantation,” Journal of Clinical Virology 76:4-7; Dieplinger, G., Everly, M. J., Briley, K. P., Haisch, C. E., Bolin, P., Maldonado, A. Q., Kendrick, W. T., and 4 others 2015. “Onset and Progression of De Novo Donor-specific Anti-human Leukocyte Antigen Antibodies after BK Polyomavirus and Preemptive Immunosuppression Reduction,” Transplant Infectious Disease 17(6): 848-858; van den Heuvel, H., Heutinck, K. M., van der Meer-Prins, E. P., Yong, S. L., Claas, F. H., and Ten Berge, I. J. 2015. “Detection of Virus-Specific CD8+ T Cells With Cross-Reactivity Against Alloantigens: Potency and Flaws of Present Experimental Methods,” Transplantation Direct 1(10): e40; van Aalderen, M. C., Heutinck, K. M., Huisman, C., and ten Berge, I. J. 2012. “BK Virus Infection in Transplant Recipients: Clinical Manifestations, Treatment Options and the Immune Response,” Netherlands Journal of Medicine 70(4): 172-183).

Readily incorporated into a prosthetic disorder control system which includes vascular and urinary valves and can coordinate the targeted delivery of medication to the site of immune reaction-associated irritation as a therapeutic adjunct, optogenetics has applications diverse as indirect use to detect pathogens and the amelioration of dysfunction in pelvic visceral control (see, for example, Mickle, A. D., Won, S. M., Noh, K. N., Yoon, J., Meachham, K. W., and 20 others 2019. “A Wireless Closed-loop System for Optogenetic Peripheral Neuromodulation,” Online, Nature 565(7739): 361-365; Park, E. and Lee, K. S. 2019. “A New Approach to Urinary Bladder Control with Optogenetics,” Investigative and Clinical Urology 60(2): 61-63; Roche, E. T. 2019. Implanted Device Enables Responsive Bladder Control,” Nature 565(7739): 298-300).

Drugs suitable for localized control or elimination of viral infection other than polyomavirus by means of delivery through side-entry jackets include those dispersed systemically but in higher concentration, such as ganciclovir 9-[1,3-dihydroxy-2-2 propoxymethyl] guanine, which presents diverse serious side effects (see, for example, Mayoral, J. L., Loeffler, C. M., Fasola, C. G., Kramer, M. A., and 4 others 1991. “Diagnosis and Treatment of Cytomegalovirus Disease in Transplant Patients Based on Gastrointestinal Tract Manifestations,” Archives of Surgery 126(2): 202-206);

Also expedited is endoscopic dextranomer and hyaluronic acid gel injection to counteract vesicoureteric, or vesicoureteral, reflux, or if due to a congenitally anomalous junction causing a dysplastic intramural ureteral tunnel and thus flap valve at the junction, endoscopic repair (see, for example, Miyakita, H., Hayashi, Y., Mitsui, T., Okawada, M., Yoshiaki, K., and 6 others 2020. “Guidelines for the Medical Management of Pediatric Vesicoureteral Reflux,” International Journal of Urology 27(6): 480-490; Brakeman, P. 2008. “Vesicoureteral Reflux, Reflux Nephropathy, and End- stage Renal Disease,” Advances in Urology 2008:508949; Williams. G., Fletcher, J. T., Alexander, S.1, and Craig, J. C. 2008. “Vesicoureteral Reflux,” Journal of the American Society of Nephrology 19(5): 8477-862; Rabinowitz, R. 2006. “Congenital Renal and Genitourinary Anomalies,” The Merck Manual, 18th edition, page 1768; page 2434), which left untreated could lead to kidney failure, and if already formed, remove renal scarring.

Vesicoureteral reflux associated with high pressure storage in the urine is conventionally treated medicinally and/or with perineal electromyography (see, for example, Rabinowitz, R. 2006, Op cit., page 2435). Thus, in this way, even nephrotic syndrome and reflux nephropathy can be effectively treated without a delay of several weeks during which inadequate treatment allowed the condition to worsen. Severe vesicoureteric reflux can be corrected laparoscopically, possibly robotic, or an open surgical Cohen, Leadbbetter-Politano, or Lich-Gregoir Technique (see, for example, Miyakita, H., Hayashi, Y., Mitsui, T., Okawada, M., Kinoshita, Y., and 6 others 2020, Op cit., preceding paragraph; Williams, G., Fletcher, J. T., Alexander, S. I., and Craig, J. 2008. “Vesicoureteral Reflux,” Journal of the American Society of Nephrology 19(5): 847-862). A serious condition could take advantage of the delay to advance upward and into the kidneys.

To allow examination of both ureters, the port has two entry openings followed by tubes leading to the ductus side-entry jacket on either ureter. Providing separate lines eliminates the nuisance of steering the cabled device from one ureter to the other between branches of a single tube. The arrangement depicted in FIG. 30 allows the patient with an intact if defective urinary tract to turn urinary diversion on or off at any time—a capability useful in many circumstances for reasons both medical and nonmedical and while diversion is possible on a temporary basis in the clinic, voluntary diversion at any moment is impossible to provide by any other means.

Whether to place such a system is a clinical judgment based upon the medical condition as much if not more so than the practical situation and choice of the patient. In contrast, the arrangement depicted in FIG. 28 is not adjustable but rather a prosthesis to be placed following a cystectomy, trauma that destroyed the bladder, or malformity of the lower tract due to a congenital defect. This system allows the patient to divert outflow into a collection bag or urinal usually cinched about a thigh for any of a large number of reasons, such as urge sensation when indisposed, a bathroom is inaccessible, or the patient is on view by the public.

Its advantages over the construction of an ileal conduit with stoma are basic and numerous, to include the elimination of a segment of small intestine as a prerequisite procedure susceptible to all the risks of a separate operation, the use of gut to pass through urine which will eventually erode and inflame the lining, and if protected by a plastic liner type stent, will do so at the inevitable leaks at its ends, and will necessitate constant maintenance of the outlet, or stoma, to prevent irritation and infection.

The prosthesis is, moreover, constantly monitored for the need of medication, which the automatic prosthetic disorder response system targets to the trouble spot, so that sources of discomfort are interdicted before these generate an experiential correlate. By comparison, surgical diversion is not practicably reversible but essentially permanent, and cannot be turned off at will. The controllable embodiment shown in FIG. 30 could be withdrawn with much less trauma but a desire to remove it much less remove it for cause, would be unusual.

Various diagnostic and surgical procedures would be more easily and effectively accomplished with the bladder empty of urine for as long as necessary. This arrangement can be combined with the creation of an anterograde path from beneath the ureteropelvic junction as addressed above to allow the passage of a miniature cabled device such as a laser or scope to be passed down the ureter and into the bladder to allow the bladder to heal without being continuously washed over by caustic urine and perform diagnostic or therapeutic procedures in a dry field. Ulceration of the bladder urothelium due to interstitial cystitis (since redesignated chronic bladder pain syndrome) is an example of a condition more effectively and efficiently treated without the constant flow of urine.

The wider application of synthetic connectors makes possible the avoidance of ileal conduits and abdominal stoma, for example. The benefits in using synthetic materials are several and important: Whether harvested, reassigned in function, or reconfigured, native tissue need not be used, eliminating the risks of complications that result from the creation of a preliminary wound to procure the tissue, such as metaplastic degeneration, infection, the removal of the blood supply and drainage at the harvesting site so that taking the internal thoracic arteries results in atrophy of the breasts. Unless pedicled, the site to which the harvested tissue is transferred must provide profusion. Often the impact on the harvesting site poses more postoperative problems than does the reconstruction.

Demanding normal percipience and voluntary control over movement, a dementia such as in α-synucleinopathic parkinsonism, or multiple system atrophy, which impairs movement, cognition, and autonomic function, invariably causes urinary incontinence for any, but usually all of these reasons (see, for example, Griebling, T. L. 2020. “Aging and Geriatric Urology,” Chapter 88 in Partin, A. W., Peters, C. A., Kavoussi, L. R., and Dmochowski, R. R. (eds.), Campbell Walsh Wein Urology, Philadelphia, Pennsylvania/St. Louis, Missouri: Elsevier Saunders; Low, P. 2006. “Autonomic Nervous System,” in The Merck Manual, 18th edition, page 1768; Resnick, N. M. and Dosa, D. 2005. “Geriatric Medicine,” in Kasper, D. L, Braunwald, E., Fauci, A. S., Hauser, S. L., Longo, D. L., and Jameson, J. L. Harrison's Principles of Internal Medicine, Chapter 8, pages 48-49, and 2413, New York, New York: McGraw-Hill Medical Publishing Division).

For such a globally destructive disease, a co-implanted automatic disorder response system able to directly pipeline target drugs to the source of any of all of these multiple incapacities in a patient incapable of adherence to a prescription regimen is decidedly beneficial. When the urinary tract is normal but voiding must be deferred to allow public performance, for example, the draining and storage apparatus to be described provides voluntary controls. Such a system with control knobs connected to Bowden push/pull cables, for example, for opening and closing the mechanical ureteral tap valves is shown in FIG. 30.

In the accompanying drawing figures, the native bladder is omitted as switched to where preserved or absent. For users capable of normal voiding but in need of a means for deferring it, the implanted system is provided with controls to allow its use on a voluntary basis. Whether the result of a congenital defect, injury, or the need for surgical removal of the lower tract, for patients with a defective lower tract and incapable of normal voiding, the system constitutes a prosthesis for which an ability to switch it off in order to allow normal voiding is impertinent.

Since the placement of an embodiment without controls in a user capable of normal voiding would specifically eliminate that capability, this is never done. Especially important for those entrusted with care of the very young, the demented, or the deranged is that an automatic collection system be easily and quickly sterilized. The systems shown in FIGS. 28 and 30 are easily sterilized by injecting an antiseptic such as an alcohol, conveniently in the form of a hand sanitizer, upstream through body surface port opening 121 and into either or both accessory channels 8 or retrograde through drainage pipe 110 to flush out pathogens or a biofilm, or an accumulation of crystal removed with a solvent.

That mechanical controls could be replaced with electrical valves is obvious, but this is less desirable as it would create the need for batteries larger than needed merely to power the implanted microcontroller. Polyuria due to diabetes is unembarrassing but a significant nuisance best dispelled with paracorporeal collection and the automatic detection of the blood glucose level and release of insulin directly into the portal vein. An intermittent need for voluntary control over the collection system pertains not only to musicians, athletes, and public speakers but to certain medical conditions as well.

Intermittent delirium, symptomatic urinary tract infection, the consumption of alcohol, or the use of any one or more of a large number of drugs more likely to be needed by the elderly, to include anxiolytics, antipsychotics, anticholinergics (known to promote dementia), potent diuretics such as furosemide (ototoxic as well, especially in veterinary use), thiazolidinediones, calcium channel blockers such as nifedipine, and Cox 2 nonsteroidal anti-inflammatory drugs such as celecoxib among others (Resnick, N. M. and Dosa, D. 2005, Op cit. above in this section).

However, urinary incontinence usually not transient (acute, intermittent, episodic) but rather chronic, involuntary discharge where control by the patient would be gratuitous, so that, as shown in FIG. 28, a system placed to counteract the constant lack of control over voiding omits controls. It is, in fact, a prosthesis suitable, for example, for placement in a female having undergone pelvic evisceration for metastatic cancer. The adverse side effects associated with the systemic dispersal of antimuscarinics and anticholinergics to dispel lower urinary tract symptoms such as nocturia, or nycturia, whether due to an overactive bladder, to include dementia as the blood brain barrier becomes degraded and more permeable with age is a classic example of the fundamental advantages to be gained from the direct pipeline targeting of drugs to the source of a disorder.

Having been dispersed in the systemic circulation with the intention of ameliorating symptoms at the bladder, the increasing permeability of the blood brain barrier with age renders it less and less effective at isolating the brain and central nervous system from potentially harmful substances in the circulation such as antimuscarinics, and especially from a lipophilic tertiary amine antimuscarinic such as oxybutynin. Concomitant causes for cognitive decline include a “significant age-related decrease in cholinergic neurons/receptors in the brain, reduction in hepatic and renal clearance of medications, and increase in blood-brain barrier (BBB) permeability . . . make older people particularly sensitive to anticholinergics In addition, the older patient population is likely to be receiving polypharmacy including other drugs with anticholinergic activity (e.g., tricyclic antidepressants, bronchodilators, ACE inhibitors, and antipsychotics), potentially resulting in a problematic anticholinergic burden, with negative effects on cognitive performance” (Griebling, T. L., Campbell, N. L., Mangel, J., Staskin, D., Herschom, S., Elsouda, D., and Schermer, C. R. 2020. “Effect of Mirabegron on Cognitive Function in Elderly Patients with Overactive Bladder: moCA [Montreal Cognitive Assessment] Results from a Phase 4 Randomized, Placebo-controlled Study (PILLAR) [arbitrary term], Online, BioMed Central Geriatrics 20(1): 1).

Numerous papers corroborate this view (see, for example, Suh, Y., Ah, Y.-M., Han E., Jun, Kwanghee, Hwang, S., Choi, K. H., and Lee, J.-Y 2020. “Dose Response Relationship of Cumulative Anticholinergic Exposure with Incident Dementia: Validation Study of Korean Anticholinergic Burden Scale,” Online, BioMed Central Geriatrics 20(1): 265; Wang, Y.-C., Chen, Y.-L., Huang, C.-C., Ho, C.-H., Huang, Y.-T., and 4 others 2019. “Cumulative Use of Therapeutic Bladder Anticholinergics and the Risk of Dementia in Patients with Lower Urinary Tract Symptoms: A Nationwide 12-year Cohort Study,” Online, BioMed Central Geriatrics 19(1): 380; American Geriatrics Society Beers Criteria Update Expert Panel 2015. “American Geriatrics Society 2015 Updated Beers Criteria for Potentially Inappropriate Medication Use in Older Adults,” Journal of the American Geriatrics Society 63(11): 2227-2246; Gray, S. L., Anderson, M. L., Dublin, S., Hanlon, J. T., Hubbard, R., and 5 others 2015. “Cumulative Use of Strong Anticholinergics and Incident Dementia: A Prospective Cohort Study,” Journal of the American Medical Association Internal Medicine 175(3): 401-407 [see also comments in Wein, S. L., 2015. “Re: Cumulative Use of Strong Anticholinergics and Incident Dementia: A Prospective Cohort Study,” Journal of Urology (Philadelphia, Pennsylvania) 193(6): 2035; Campbell, N., Boustani, M., Limbil. T., Ott. C., Fox, C., and 6 others 2009. “The Cognitive Impact of Anticholinergics: A Clinical Review,” Clinical Interventions in Aging Online 4:225-233). Griebling. Campbell, et al. 2020 (Op cit. above in the preceding paragraph), point out that antimuscarinics can differ significantly in the extent to which each may impair higher mental function.

Limited to the oral route, the hydrophilic muscarinic antagonist quaternary amine trospium chloride, because it is less likely to cross the barrier, is preferable to oxybutynin (Geller, E. J., Dumond, J. B., Bowling, J. M., Khandelwal C. M., Wu, J. M., Busby-Whitehead, J., and Kaufer, D. I. 2017. “Effect of Trospium Chloride on Cognitive Function in Women Aged 50 and Older: A Randomized Trial,” Female Pelvic Medicine and Reconstructive Surgery 23(2): 118-123; Chancellor, M. and Boone, T. 2012. “Anticholinergics for Overactive Bladder Therapy: Central Nervous System Effects,” CNS Neuroscience and Therapeutics 18(2): 167-174; Staskin, D., Kay, G., Tannenbaum, C., Goldman, H. B., Bhashi, K., Ling. J., and Oefelein, M. G. 2010. “Trospium Chloride Has No Effect on Memory Testing and is Assay Undetectable in the Central Nervous System of Older Patients with Overactive Bladder,” International Journal of Clinical Practice 64(9): 1294-1300). As the blood brain barrier continues to increase in permeability, the ligation of acetylcholine to muscarinic neuronal transmembrane receptors M1 thru M5 in the brain and central nervous system likewise increases until the side effects rise to the clinical.

Much research has gone into the degradation in cognitive function caused by specific and that comparative as between different antimuscarinics (see, for example, Dantas, L. P., Forte, A. R. C. C., Lima, B. C., Sousa, C. N. S., Vasconcelos, E. C., and 4 others 2022. “Treatment of Bladder Dysfunction with Solifenacin: Is there a Risk of Dementia or Cognitive Impairment?,” Brazilian Journal of Medical and Biological Research Online 55: e11721; Welk, B., Richardson, K., and Panicker, J. N. 2021. “The Cognitive Effect of Anticholinergics for Patients with Overactive Bladder,” Nature Reviews Urology 18(11): 686-700; Araklitis, G., Robinson, D., and Cardozo, L. 2020. “Cognitive Effects of Anticholinergic Load in Women with Overactive Bladder,” Online Clinical Interventions in Aging 15:1493-1503; Kim, Y. J., Tae. B. S., and Bae, J. H. 2020. “Cognitive Function and Urologic Medications for Lower Urinary Tract Symptoms,” International Neurourology Journal 24(3): 231-240; Wagg. A., Arumi. D., Herschom, S., Angolo Cuesta, J., Haab, F., and 3 others 2017. “A Pooled Analysis of the Efficacy of Fesoterodine for the Treatment of Overactive Bladder, and the Relationship between Safety, Co-morbidity, and Polypharmacy in Patients Aged 65 Years or Older,” Age and Ageing 46(4): 620-626; Oelke, M., Murgas, S., Schneider, T., and HeBdörfer, E. 2013. “Influence of Propiverine ER [extended release] 30 mg Once Daily on Cognitive Function in Elderly Female and Male Patients with Overactive Bladder: A Non-interventional Study to Assess Real Life Data,” Neurourology and Urodynamics 32(6): 800-802; Wagg. A., Dale, M., Tretter, R., Stow. B., and Compion, G. 2013. “Randomised, Multicentre, Placebo-controlled, Double- blind Crossover Study Investigating the Effect of Solifenacin and Oxybutynin in Elderly People with Mild Cognitive Impairment: the SENIOR [meaning in kind] Study,” European Urology 64(1): 74-81; Oelke, M., Murgas, S., Baumann, I., Chnabel, F., and Michel, M. C. 2011. “Efficacy of Propiverine ER [extended release] with or without Alpha-blockers Related to Maximum Urinary Flow Rate in Adult Men with OAB [overactive bladder]: Results of a 12-week, Multicenter, Noninterventional Study,” World Journal of Urology 29(2): 217-223; Wesnes, K. A., Edgar, C., Tretter, R. N., and Bolodeoku, J. 2009. “Exploratory Pilot Study Assessing the Risk of Cognitive Impairment or Sedation in the Elderly following Single Doses of Solifenacin 10 mg,” Expert Opinion on Drug Safety 8(6): 615-626; Kay, G., Crook, T., Rekeda, L., Lima, R., Ebinger, U., and Arguinzoniz, M. 2006. “Differential Effects of the Antimuscarinic Agents Darifenacin and Oxybutynin ER [extended release] on Memory in Older Subjects,” European Urology (Amsterdam, North Holland, Netherlands) 50(2): 317-326 Kay, G., Kardiasmenos, K., and Crook, T. 2006. Differential Effects of the Antimuscarinic Agents Tolterodine Tartrate ER [extended release] and Oxybutynin Chloride ER [extended release] on Recent Memory in Older Subjects,” in Proceedings of the 36th Meeting of the International Continence Society 25(6): P087).

In the elderly, the side effects caused by anticholinergics can cause constipation; particularly in men with benign prostatic hyperplasia, urinary retention; especially in those with dementia, confusion and somnolence; blurred vision; orthostatic hypotension, and dry mouth (The Merck Manual, 18th edition, 2006, page 2536; Roher, A. E., Kuo, Y. M., Potter, P. E., Emmerling, M. R., Durham, and 4 others 2000. “Cortical Cholinergic Denervation Elicits Vascular Aβ [amyloid beta] Deposition,” Annals of the New York Academy of Sciences 903(1): 366-373).

Muscarinic receptors M1 and M2 associated with higher cognitive processes, antimuscarinies and anticholinergics in the elderly can also cause delirium, cognitive impairment to include short term memory loss, dementia, and increased risk of death (Araklitis, G., Robinson, D., and Cardozo, L. 2020. “Cognitive Effects of Anticholinergic Load in Women with Overactive Bladder,” Clinical Interventions in Aging 15:1493-1503; Kay, G. G. and Granville, L. J. 2005. “Antimuscarinic Agents: Implications and Concerns in the Management of Overactive Bladder in the Elderly,” Clinical Therapeutics 27(1): 127-138).

Newer drugs often prohibitively expensive, direct mechanically constrained, or pipeline targeted, delivery allows the use of doses tiny compared to those needed for dispersal throughout the circulatory system where only a small fraction will actually act as intended. At the same time, the need to limit the dose level or to preclude the combination of one drug with another may well invalidate the intrinsic efficacy of numerous drugs were this restriction removed. Thus, direct targeting supports both smaller and larger dosing. Overall, delivery by means of direct pipeline targeting is likely to liberate the dosing of a vast number of drugs used for different purposes previously enjoined from release into the general circulation at the heightened levels needed to be effective at the target.

Moreover, direct release into the target substantially reduces if not eliminates drug-drug and drug-food interactions. A means for considerably reducing if eliminating adverse side effects reduces not only the hesitancy with which drugs may be prescribed but also the vigilance with which patients must be monitored as a serious drain on the time available to medical professionals. Pipelined, the concurrent release of different drugs isolates each along a separate track, allowing the control microprocessor to administer each according to its respective prescription.

Many drugs released into the circulation produce adverse side effects, but those used to treat lower urinary tract symptoms stand out in this regard as well as in a lack of efficacy. Here the likely cause is that not targeted directly to the bladder and prohibited from release into the circulation in an adequate amount, dose sufficiency is never attained.

Accordingly, that drugs used in the treatment of urological disorders such as overactive bladder and incontinence often produce severe adverse side effects recommends the use of means to isolate such drugs from the central nervous system, or better, providing means that allow dispensing with drugs to deal with such problems altogether: “There is growing evidence that antimuscarinics may increase the risk of cognitive impairment, dementia, and even death,” (Harnod, T., Yang, Y.-C., Chiu, L.-T., Wang, J.-H., Lin, S.-Z., and Ding. D.-C. 2021. “Use of Bladder Antimuscarinics is Associated with an Increased Risk of Dementia: A Retrospective Population-based Case-control Study,” Scence Reports Online 11:4827; Araklitis, G. and Robinson, D. 2020. “The Cognitive Safety of Antimuscarinics in the Treatment of Overactive Bladder,” Expert Opinion on Drug Safety 19(10): 1303-1313; Araklitis, G., Robinson, D. and Cardozo, L. 2020. “Cognitive Effects of Anticholinergic Load in Women with Overactive Blader,” Online, Clinical Interventions in Aging 15:1493-1503); (see also; Lozano-Ortega, G., Walker, D. R., Johnston, K., Mickle, A., Harrigan, S., and 4 others 2020. “Comparative Safety and Efficacy of Treatments for Overactive Bladder among Older Adults: A Network Meta-analysis,” Drugs and Aging 37(11): 801-816; Makhani, A., Thake. M., and Gibson, W. 2020. “Mirabegron in the Treatment of Overactive Bladder: Safety and Efficacy in the Very Elderly Patient,” Clinical Interventions in Aging 15: 575-581; Ali, S., Peterson, G. M., Bereznicki, L. R., and Salahudeen, M. S. 2019. “Association between Anticholinergic Drug Burden and Mortality in Older People: A Systematic Review,” European Journal of Clinical Pharmacology December: 1-7; Coupland, C. A. C., Hill, T., Dening. T., Morriss. R. Moore, R. and Hippisley-Cox, J. 2019. “Anticholinergic Drug Exposure and the Risk of Dementia: A Nested Case-control Study Journal of the American Medical Association Internal Medicine 179(8): 1084-1093; Masuoka, T., Uwada, J., Kudo, M., Yoshiki, H., Yamashita, Y., and 4 others 2019. “Augmentation of Endogenous Acetylcholine Uptake and Cholinergic Facilitation of Hippocampal Long-term Potentiation by Acetylcholinesterase Inhibition,” Neuroscience 404:39-47; Richardson, K., Fox. C., Maidment, I., Steel, N., Loke, Y. K., and 11 others 2018. “Anticholinergic Drugs and Risk of Dementia: Case-control Study,” Online, British Medical Journal 361: k1315; Triantafylidis, L. K., Clemons, J. S., Peron, E. P., Roefaro, J., and Zimmerman, K. M. 2018. “Brain over Bladder: A Systematic Review of Dual Cholinesterase Inhibitor and Urinary Anticholinergic Use,” Drugs and Aging 35(1): 27-41; Wagg. A. 2018. “Anticholinergics for Overactive Bladder in Frail and Medically Complex Older People: The Case for,” Drugs and Aging 35(9): 777-780: Woodford, H. J. 2018. “Anticholinergic Drugs for Overactive Bladder in Frail Older Patients: The Case Against,” Drugs and Aging 35(9): 773-776; Araklitis, G. and Cardozo, L. 2017. “Safety Issues Associated with Using Medication to Treat Overactive Bladder,” Expert Opinion on Drug Safety 16(11): 1273-1280; Thomas, T. N. and Walters, M. D., eds. 2017. “AUGS [American Urogynecologic Society] Consensus Statement: Association of Anticholinergic Medication Use and Cognition in Women with Overactive Bladder,” Female Pelvic Medicine and Reconstructive Surgery 23(3): 177-178; Risacher, S. L., McDonald, B. C., Tallman, E. F., West. J. D., Martin R Farlow, M. R., and 10 others 2016. “Association between Anticholinergic Medication Use and Cognition, Brain Metabolism, and Brain Atrophy in Cognitively Normal Older Adults,” Journal of the American Medical Association Neurology 73(6): 721-732; Wagg. A., Nitti, V. W., Kelleher. C., Castro-Diaz, D., Siddiqui, E., and Berner, T. 2016. “Oral Pharmacotherapy for Overactive Bladder in Older Patients: Mirabegron as a Potential Alternative to Antimuscarinics,” Current Medical Research and Opinion 32(4): 621-638; Oelke M., Becher, K., Castro-Diaz, D., Chartier-Kastler, E., Kirby, M., Wagg. A., and Wehling, M. 2015. “Appropriateness of Oral Drugs for Long-term Treatment of Lower Urinary Tract Symptoms in Older Persons: Results of a Systematic Literature Review and International Consensus Validation Process (LUTS-FORTA 2014} [lower urinary tract symptoms-Fit FOR The Aged],” Age and Ageing 44(5): 745-755; Fox, C., Smith, T., Maidment, I., Chan, W.-E., Bua, N. and 6 others 2014. “Effect of Medications with Anti-cholinergic Properties on Cognitive Function, Delirium, Physical Function, and Mortality: A Systematic Review,” Age and Ageing 43(5): 604-615; Chancellor, M. and Boone, T. 2012. “Anticholinergics for Overactive Bladder Therapy: Central Nervous System Effects,” CNS [Central Nervous System] Neuroscience and Therapeutics 18(2): 167-174; Chancellor, M. B., Staskin, D. R., Kay, G. G., Sandage, B. W., Oefelein, M. G., and Tsao. J. W. 2012. “Blood-brain Barrier Permeation and Efflux Exclusion of Anticholinergics Used in the Treatment of Overactive Bladder,” Drugs and Aging 29(4): 259-273; Fox, C., Richardson, K., Maidment, I. D., Savva, G. M., Matthews, F. E., and 5 others 2011. “Anticholinergic Medication Use and Cognitive Impairment in the Older Population: The Medical Research Council Cognitive Function and Ageing Study,” Journal of the American Geriatrics Society 59(8): 1477-1483).

Electrostimulatory neuromodulation appears to be more effective than nontargeted drugs, stimulation of the sacral more effective than the tibial nerve but it too does not afford adequate relief (see, for example, Al-Danakh, A., Safi. M., Alradhi, M., Almoiliqy. M., Chen, Q., and 5 others 2022. “Posterior Tibial Nerve Stimulation for Overactive Bladder: Mechanism, Classification, and Management Outlines, Online, Parkinson's Disease 2022:2700227; Enemchukwu, E. A., Subak, L. L., and Markland, A. 2022. “Barriers and Facilitators to Overactive Bladder Therapy Adherence,” Neurourology and Urodynamics 41(8): 1983-1992; Feloney, M. P., Strauss, K., and Leslie, S. W. 2022. “Sacral Neuromodulation,” Online, Treasure Island, Florida: StatPearls Publishing Co.; Fernandez-Cuadros, M. E., Martin-Martin, L. M., Albaladejo-Florin, M. J., Perez-Moro, O. S., Alava-Rabasa, S., and Goizueta-San Martin, G. 2022. “Transcutaneous Simulation of the Posterior Tibial Nerve Modifies the Sympathetic Skin Response and Improves Overactive Bladder Syndrome: Case Series and Possible Diagnostic Test,” (Spanish with English abstract) Rehabilitacion 56(4): 255-263; Jairam, R., Drossaerts. J., Marcelissen, T., van Koeveringe, G., Vrijens. D., and van Kerrebroeck, P. 2022. “Predictive Factors in Sacral Neuromodulation: A Systematic Review,” Online, Urologia Internationalis 106(4): 323-343; Khasanah, N., Chin, H. Y., and Peng. C. W. 2022. “Physical Agent-based Treatments for Overactive Bladder: A Review,” Online, MDPI [Multidisciplinary Digital Publishing Institute] Journal of Clinical Medicine (Basel, Switzerland) 11(17): 5150; Krhut, J., Rejchrt, M., Skugarevska, B., Grepl. M., and Zvara, P. 2022. “Peroneal Electrical Transcutaneous Neuromodulation as a New Treatment for Patients with Overactive Bladder: An Initial Clinical Experience,” Urologia Internationalis 106(7): 658-663; Loloi, J., Clearwater, W., Schulz, A., Suadicani, S. O., and Abraham, N. 2022. “Medical Treatment of Overactive Bladder,” Urologic Clinics of North America 49(2): 249-261; Tilborghs, S., and De Wachter, S. 2022. “Sacral Neuromodulation for the Treatment of Overactive Bladder: Systematic Review and Future Prospects,” Online, Expert Review of Medical Devices 19(2): 161-187; Yuan, X. Balog, B. M., Lin, D. L., Hanzlicek, B., Kuang. M., and 3 others 2022. “Brain-derived Neurotrophic Factor is an Important Therapeutic Factor in Mesenchymal Stem Cell Secretions for Treatment of Traumatic Peripheral Pelvic Injuries,” Online, Frontiers in Cellular Neuroscience 16:866094; Zhong, H., Liu, E., Kohli, P., Peres. L., Edgerton, V. R., and 3 others 2022. “Noninvasive Spinal Neuromodulation Mitigates Symptoms of Idiopathic Overactive Bladder,” Online, BioMed Central Bioelectronic Medicine 8(1): 5; Zoqlam, R., Lasauskaite, S., Glickman, S., Zaitseva, L., Ilie, P.-C., and Sheng, Q. 2022. “Emerging Molecular Mechanisms and Genetic Targets for Developing Novel Therapeutic Strategies for Treating Bladder Diseases,” Online, European Journal of Pharmaceutical Sciences 173:106167; Balog, B. M., Deng. K., Askew, T., Kuang, M., Hanzlicek. B., and Damaser, M. S. 2021. “Brain Derived Neurotrophic Factor Mediates Accelerated Recovery of Regenerative Electrical Stimulation in an Animal Model of Stress Urinary Incontinence,” Experimental Neurology 343:113781; Feldkamp, A., Amasyalι, S., Groegler, J., Jellison, F., and Staack A. 2021. “Evaluation of Progression from First to Second Stage Sacral Neuromodulation and Unplanned Device Removal,” Turkish Journal of Urology 47(1): 58-65; Krhut, J., Peter. L., Reichrt, M., Slovak, M., Skugarevska, B., and Zvara, P. 2021. “Peroneal Electric Transcutaneous NeuroModulation (eTNM®): A Novel Method for the Treatment of the Overactive Bladder,” Online, Healthcare Engineering 2021:4016346; Mass-Lindenbaum, M., Calderon-Pollak, D., Goldman, H. B., and Pizarro-Berdichevsky, J. 2021. “Sacral Neuromodulation-When and for Who,” Online, International Brazilian Journal of Urology 47(3): 547-656; Balog, B. M., Askew. T., Lin, D. L., Kuang. M., Habzlicek, B., and Damaser, M. S. 2020. “The Pudendal Nerve Motor Branch Regenerates via a Brain Derived Neurotrophic Factor Mediated Mechanism,” Online, Experimental Neurology 334:113438; De Wachter, S., Knowles, C. H., Elterman, D. S., Kennelly, M. J., Lehur, P. A., and 3 others 2020. “New Technologies and Applications in Sacral Neuromodulation: An Update,” Online, Advances in Therapy 37(2): 637-643; Langdale, C. L. Hokanson, J. A., Milliken, P. H. Sridhar, A. and Grill, W. M. 2020. “Stimulation of the Pelvic Nerve Increases Bladder Capacity in the PGE: [prostaglandin E2] Cat Model of Overactive Bladder,” Online, American Journal of Physiology. Renal Physiology 318(6): F1357-F1368; Lo, C. W., Wu, M. Y., Yang, S. S., Jaw, F. S., and Chang, S. J. 2020. Comparing the Efficacy of OnabotulinumtoxinA, Sacral Neuromodulation, and Peripheral Tibial Nerve Stimulation as Third Line Treatment for the Management of Overactive Bladder Symptoms in Adults: Systematic Review and Network Meta-analysis,” Toxins (Basel, Switzerland). 12(2): 128; Vollstedt, A. and Gilleran, J. 2020. “Update on Implantable PTNS [percutaneous tibial nerve stimulation] Devices,” Current Urology Reports 21(7): 28; Adelstein, S. A., Lee, W., Gioia, K., Moskowitz, D., Stamnes, K., and 3 others 2019. “Outcomes in a Contemporary Cohort Undergoing Sacral Neuromodulation Using Optimized Lead Placement Technique,” Neurourology and Urodynamics 38(6): 1595-1601; Balog. B. M., Deng. K., Labhasetwar, V., Jones, K. J., and Damaser, M. S. 2019. “Electrical Stimulation for Neuroregeneration in Urology: A New Therapeutic Paradigm,” Online, Current Opinion in Urology 29(4): 458-465; Chen, L. C. and Kuo, H. C. 2019. “Pathophysiology of Refractory Overactive Bladder,” Lower Urinary Tract Symptoms 11(4): 177-181; Coolen, R. L., Groen, J., and Blok, B. F. M. 2019. “Electrical Stimulation in the Treatment of Bladder Dysfunction: Technology Update,” Online, Medical Devices (Auckland, New Zealand) 12:337-345; Moore, C. K., Rueb, J. J., and Derisavifard, S. 2019. “What is New in Neuromodulation?” Current Urology Reports 20(9): 55; Palmer, C., Farhan, B., Nguyen, N., and Ghoniem G. 2019. “Clinical Outcomes of Percutaneous Tibial Nerve Stimulation in Elderly Patients with Overactive Bladder,” Arab Journal of Urology 17(1): 10-13; Peyronnet, B., Mironska, E., Chapple, C., Cardozo, L. Oelke, M., and 6 others 2019. “A Comprehensive Review of Overactive Bladder Pathophysiology: On the Way to Tailored Treatment” (see also multiple comments) European Urology 75(6): 988-1000; Szymanski, J. K., Slabuszewska-Jozwiak, A., Zareba, K., and Jakiel, G. 2019. “Neuromodulation-A Therapeutic Option for Refractory Overactive Bladder, A Recent Literature Review,” (in English), Online, Videosurgery and Other Miniinvasive Techniques 14(4): 476-485: Tahseen, S. 2018. “Role of Sacral Neuromodulation in Modern Urogynaecology Practice: A Review of Recent Literature,” International Urogynecology Journal 29(8): 108-1091; Tutolo, M., Ammirati, E., Heesakkers, J., Kessler, T. M., Peters, K. M., and 6 others 2018. “Efficacy and Safety of Sacral and Percutaneous Tibial Neuromodulation in Non-neurogenic Lower Urinary Tract Dysfunction and Chronic Pelvic Pain: A Systematic Review of the Literature,” European Urology 73(3): 406-418; Faris, A. E. R. Gill, B. C., Pizarro-Berdichevsky, J., Dielubanza, E., Clifton, M. M., and 5 others 2017. “Impact of Age and Comorbidities on Use of Sacral Neuromodulation,” Journal of Urology (Philadelphia, Pennsylvania) 198(1): 161-166; Jairam, R., Marcelissen, T., van Koeveringe. G., and van Kerrebroeck, P. 2017. “Optimal Lead Positioning in Sacral Neuromodulation: Which Factors are Related to Treatment Outcome?,” Neuromodulation 20(8): 830-835; Sukhu, T., Kennelly, M. J., and Kurpad, R. 2016. “Sacral Neuromodulation in Overactive Bladder: A Review and Current Perspectives,” Online, Research and Reports in Urology 8:193-199; Balchandra, P. and Rogerson L. 2014. “Women's Perspective: Intra-detrusor Botox versus Sacral Neuromodulation for Overactive Bladder Symptoms after Unsuccessful Anticholinergic Treatment,” International Urogynecology Journal 25(8): 1059-1064; van Kerrebroeck, P. E. V. 2010. “Advances in the Role of Sacral Nerve Neuromodulation in Lower Urinary Symptoms, Online, International Urogynecology Journal 21 (Supplement 2): 467-474; White, W. M., Mobley, III J. D., Doggweiler, R., Dobmeyer-Dittrich, C., and Klein, F. A. 2009. “Sacral Nerve Stimulation for Refractory Overactive Bladder in the Elderly Population,” Journal of Urology (Philadelphia, Pennsylvania) 182(4): 1449-1452; Leng, W. W. and Chancellor, M. B. 2005. “How Sacral Nerve Stimulation Neuromodulation Works,” Urologie Clinics of North America 32(1): 11-18; Amundsen, C. L. and Webster, G. D. 2002. “Sacral Neuromodulation in an Older, Urge-incontinent Population,” American Journal of Obstetrics and Gynecology 187(6): 1462-1465).

Antimuscarinics or anticholinergics at a higher dose than should be allowed to circulate can be directly pipeline targeted for automatic release to the bladder by placement of basic ductus side-entry jackets along the internal iliac (hypogastric) arteries for continued flow through the inferior vesicle arteries. That at most a trace amount would enter the general circulation should eliminate concern over cognitive impairment. A basic ductus side-entry jacket for use solely of its accessory channel as a drugline supply connector is a passive device with an integral as co-molded, hence, fixed, diversion chute, which not a valve and nonadjustable, has no side stem.

With this flow diversion device, only an accessory channel connected by a catheter to the implanted drug supply reservoir used. If not obtrusive to neighboring tissue, any ductus side-entry device can be used for fixed flow diversion out through the sidestem when fully extended as shown in the drawing FIGS. 2, 5, 7, 8, and 10A. Unlike a simple fixed-as-molded jacket, these are valves which can adjust the volume of outflow. When the diversion chute is fully retracted, the outlet passage through the side stem is closed off so that flow continues past the outlet ostium through the substrate ductus.

The system components to target these drugs and minimize if not eliminate their potentially adverse side effects can be placed endoscopically through small incisions, the drug supply catheter tunneled subcutaneously to the drug reservoir of which the outlet is controlled by an implanted microcontroller, or in the case of comorbid disease, a hierarchical control system master control microprocessor. Compared to systemic dispersal, the direct targeting of the bladder with an antimuscarinic or anticholinergic at a dose level not restricted due to concerns about side effects should more effectively ameliorate if not eliminate vexatious bladder symptoms. However, only a totally implanted system can achieve the elimination of symptoms while providing complete control over voiding.

Drugs touted as avoiding the side effects of antimuscarinics and anticholinergics have their own potentially serious side effects. Mirabegron with or without the addition of solifenacin can provoke dangerously elevated blood pressure, severe headache, dizziness, sinus, joint, chest, stomach, urinary bladder, and/or back pain, nephrolithiasis, urinary retention, constipation, symptoms ordinarily associated with a cold or the flu, and when taken with solifenacin, dry mouth, constipation, xerostomia (‘dry mouth’), tachycardia, arrhythmia, painful or difficult urination, tinnitus, apprehension, confusion, dyspnea, seizures, and less commonly, blurred vision, glaucoma, indigestion, gastritis, Stevens-Johnson syndrome, and an elevation in liver enzymes.

Complications associated with the use of antimuscarinic and anticholinergic avoidance drugs is well documented (see, for example, (Griebling, T. L., Campbell, N. L., Mangel, J., Staskin. D., Herschom, S., Elsouda, D., and Schermer, C. R. 2020. Op cit. ahove in this section; Herschorn, S., Staskin, B., Schermer, C. R. Kristy, R. M., and Wagg. A. 2020. “Safety and Tolerability Results from the PILLAR [undefined] Study: A Phase IV. Double-Blind, Randomized, Placebo-controlled Study of Mirabegron in Patients >65 Years with Overactive Bladder-Wet,” Drugs and Aging 37(9): 665-676; Wagg. A., Staskin, D., Engel, E., Herschorn, S., Kristy, R. M., and Schermer, C. R. 2020. “Efficacy, Safety, and Tolerability of Mirabegron in Patients Aged >65 Yr with Overactive Bladder Wet: A Phase IV, Double-blind, Randomised, Placebo-controlled Study (PILLAR) [undefined], European Urology 77(2): 211-220; Yoshida, M., Nozawa, Y., Kato, D., Tabuchi, H., and Kuroishi, K. 2019. “Safety and Effectiveness of Mirabegron in Patients with Overactive Bladder Aged >75 Years: Analysis of a Japanese Post-marketing Study,” Lower Urinary Tract Symptoms 11(1): 30-38; Drake, M. J., MacDiarmid, S., Chapple, C. R., Esen, A., Athanasiou, S., and 6 others 2017. “Cardiovascular Safety in Refractory Incontinent Patients with Overactive Bladder Receiving Add-on Mirabegron Therapy to Solifenacin (BESIDE) [undefined],” International Journal of Clinical Practice 71(5): e12944; Wagg. A., Nitti, V. W., Kelleher, C., Castro-Diaz, D., Siddiqui, E., and Bemner, T. 2016, Op cit. above in this section; Balchandra, P. and Rogerson L. 2014, Op cit. above in this section; Wagg. A., Cardozo, L., Nitti, V. W., Castro-Diaz, D., Auerbach, S., Blauwet, M. B., and Siddiqui, E. 2014. “The Efficacy and Tolerability of the β3-adrenoceptor Agonist Mirabegron for the Treatment of Symptoms of Overactive Bladder in Older Patients,” Age and Ageing 43(5): 666-675: Tyagi. P., Tyagi. V., and Chancellor, M. 2011. “Mirabegron: A Safety Review” Expert Opinion on Drug Safety 10(2): 287-289).

OnabotulinumtoxinA detrusor injections have been widely reported to be effective (see, for example, Schulte-Baukloh, H., Weiß, C., Weinberger, S., Hubatsch, M., Schlomm, T., and Ralla, B. 2022. “Real-time Documentation of the Effect of OnabotulinumtoxinA Detrusor Injection in OAB [overactive bladder] Patients-Preliminary Results,” Online, MDPI [Multidisciplinary Digital Publishing Institute] Toxins (Basel, Switzerland) 15(1): 30; Onem, K., Bayrak, O., Demirtas, A., Coskun, B., Dincer, M., Kocak, I., and Onur, 2018. “Efficacy and Safety of OnabotulinumtoxinA Injection in Patients with Refractory Overactive Bladder: First Multicentric Study in Turkish Population,” Neurourology and Urodynamics (New York, New York) 37(1): 263-268; Eldred-Evans. D. and Sahai, A. 2017. “Medium-to Long-term Outcomes of Botulinum Toxin A for Idiopathic Overactive Bladder,” Online, Therapeutic Advances in Urology (London, England) 9(1): 3-1: Chapple, C., Sievert, K. D., MacDiarmid, S., Khullar, V., Radziszewski, P., and 4 others 2013. “OnabotulinumtoxinA 100 U Significantly Improves All Idiopathic Overactive Bladder Symptoms and Quality of Life in Patients with Overactive Bladder and Urinary Incontinence: A Randomised, Double-blind, Placebo-controlled Trial,” European Urology (Basel, Switzerland) 64(2): 249-256; Madersbacher, S. 2013. “OnabotulinumtoxinA for Idiopathic Overactive Bladder Symptoms: Many Answers but More Questions,” [response to Chapple, et al. preceding reference], European Urology (Basel, Switzerland) 64(2): 257-259.).

Giving emphasis to the relative freedom from noniatrogenic adverse consequences of implanting a system such as that shown in FIG. 30, these encouraging results notwithstanding, the side effects of botulinum toxin injected intravesically has also been reported to be capable of inducing serious side effects, to include dysphagia, arrhythmias, heart attack, seizures, respiratory arrest, muscle weakness, inordinate post-voiding retention of urine requiring catheterization, urinary tract infections, allergic reactions, flu-like syndromes, and death (see, for example, Eldred-Evans, D. and Sahai. A. 2017. “Medium-to Long-term Outcomes of Botulinum Toxin A for Idiopathic Overactive Bladder,” Therapeutic Advances in Urology 9(1): 3-10; Jiang, Y. H., Ong. H. L., and Kuo, H. C. 2017. “Predictive Factors of Adverse Events after Intravesical Suburothelial OnabotulinumtoxinA A Injections for Overactive Bladder Syndrome—A Real-life Practice of 290 Cases in a Single Center, Neurourology and Urodynamics 36(1): 142-147; Miotla, P., Cartwright, R., Skorupska, K., Bogusiewicz, M., Markut-Miotla, E., Futyma. K., and Rechberger, T. 2017. “Urinary Retention in Female OAB after Intravesical Botox Injection: Who is Really at Risk?,” International Urogynecology Journal 28(6): 845-850; Hsieh, P.-F., Chiu, H.-C., Chen, K. C., Chang, C-H., and Chou, E. C-L. 2016. “Botulinum Toxin A for the Treatment of Overactive Bladder,” Toxins (Basel, Switzerland) 8(3): 59; Cui. Y., Zhou, X., Zong, H., Yan, H., and Zhang, Y. 2015. “The Efficacy and Safety of OnabotulinumtoxinA in Treating Idiopathic OAB: A Systematic Review and Meta-analysis,” Neurourology and Urodynamics 34(5): 413-419; Davis, N. F., Burke, J. P., Redmond, E. J., Elamin, S., Brady, C. M., and Flood, H. D. 2015. “Trigonal versus Extratrigonal Botulinum Toxin—A: A Systematic Review and Meta-analysis of Efficacy and Adverse Events,” International Urogynecology Journal 26(3): 313-319; Osborn, D. J., Kaufman, M. R., Mock, S., Guan, M. J., Dmochowski, R. R., and Reynolds. W. S. 2015. “Urinary Retention Rates after Intravesical OnabotulinumtoxinA Injection for Idiopathic Overactive Bladder in Clinical Practice and Predictors of this Outcome,” Neurourology and Urodynamics 34(7): 675-678; Zhou, X., Yan, H. L., Cui, Y. S., Zong, H. T. and Zhang, Y. 2015. “Efficacy and Safety of OnabotulinumtoxinA in Treating Neurogenic Detrusor Overractivity: A Systematic Review and Meta-analysis,” Chinese Medical Journal (English edition) 128(7): 963-968; Rovner, E. 2014. “Practical Aspects of Administration of OnabotulinumtoxinA,” Neurourology and Urodynamics 33 (Supplement 3): S32-S37. Chapter 6: Liao. C. H. and Kuo, H. C. 2013. “Increased Risk of Large Post-void Residual Urine and Decreased Long-term Success Rate after Intravesical OnabotulinumtoxinA Injection for Refractory Idiopathic Detrusor Overactivity,” Journal of Urology (Philadelphia, Pennsylvania) 189(5): 1804-1810 with comment by Smith, P. P. and Kuchel. G. A. 2013,” Editorial Comment Journal of Urology (Philadelphia, Pennsylvania) 189(5): 1810; Linsenmeyer, T. A. 2013. “Use of Botulinum Toxin in Individuals with Neurogenic Detrusor Overactivity: State of the Art Review,” Journal of Spinal Cord Medicine 36(5): 402-419; Orasanu, B. and Mah ajan, S. T. 2013. “The Use of Botulinum Toxin for the Treatment of Overactive Bladder Syndrome,” Indian Journal of Urology 29(1): 2-11: Santos-Silva, A. da Silva, C. M., and Cruz, F. 2013. “Botulinum Toxin Treatment for Bladder Dysfunction,” International Journal of Urology 20(10): 956-962; Bauer, R. M., Gratzke, C., Roosen, A. Hocaoglu, Y., Mayer, M. E., and 3 others 2011. “Patient-reported Side Effects of Intradetrusor Botulinum Toxin Type A for Idiopathic Overactive Bladder Syndrome,” Urologia Internationalis 86(1): 68-72; Duthie, J. B., Vincent, M., Herbison, G. P., Wilson, D. I., and Wilson, D. 2011. “Botulinum Toxin Injections for Adults with Overactive Bladder Syndrome,” Cochrane Database of Systematic Reviews 12: CD005493; Rovner E., Kennelly, M., Schulte-Baukloh, H., Zhou, J., Haag-Molkenteller, C., and Dagupta, P. 2011. “Urodynamic Results and Clinical Outcomes with Intradetrusor Injection of OnabotulinumtoxinA in a Randomized, Placebo-controlled Dose-finding Study in Idiopathic Overactive Bladder,” Neurourology and Urodynamics 30(4): 556-562; Dmochowski, R. Chapple. C., Nitti, V. W., Chancellor, M., Everaert, K., and 4 others 2010. “Efficacy and Safety of OnabotulinumtoxinA for Idiopathic Overactive Bladder: A Double-blind Placebo-controlled Randomized, Dose Ranging Trial,” Journal of Urology (Philadelphia, Pennsylvania) 184(6): 2416-2422; Kuo, H.-C., Liao. C.-H., and Chung, S.-D. 2010. “Adverse Events of Intravesical Botulinum Toxin A Injections for Idiopathic Detrusor Overactivity: Risk Factors and Influence on Treatment Outcome,” European Urology 58(6): 919-926; United States Food and Drug Administration 2008. “FDA Notifies Public of Adverse Reactions Linked to Botox Use;” “FDA Gives Update on Botulinum Toxin Safety Warnings; Established Names of Drugs Changed,” Pharmaceutical Online, 4 Aug. 2009; Cote, T. R., Mohan, A. K., Polder, J. A., Walton, M. K., and Braun, M. M. 2005. “Botulinum Toxin Type A Injections: Adverse Events Reported to the U.S. Food and Drug Administration in Therapeutic and Cosmetic Cases,” Journal of the American Academy of Dermatology 53(3): 407-415).

The efficacy and avoidance of adverse side effects caused by mirabegron and botulinum toxin type A can be reduced if not eliminated in the same direct pipeline-targeted manner as specified for antimuscarinics and anticholinergics. Administration thus likewise allows a considerable reduction in the doses used. While this should materially increase the efficacy and reduce the side effects of these drugs, the relief obtained will fall short of that provided by a mechanical voiding system, which eliminates these problems altogether.

Moreover, for the purpose of eradicating incontinence as a symptom, the totally implanted mechanical system eliminates the need to determine whether the condition is urge incontinence wherein uncontrollable voiding ensues with a piercing sensation of need to do so, usually in the night as nocturia; stress incontinence wherein urine is expelled with any activity that increases intra-abdominal pressure; overflow incontinence wherein there is a loss of containment when the bladder is full resulting in dribbling leakage; functional incontinence wherein due to mental or physical impairment, the patient is unable to respond to the urge; or mixed incontinence involving the foregoing in any combination (see, for example, The Merck Manual, 2006, “Voiding Disorders,” Chapter 228, page 1951, Whitehouse Station, New Jersey: Merck Research Laboratories Division, Merck and Company, Inc.).

Urinary incontinence is diagnostically problematic in that the symptoms equate to the medical complaint without regard to the etiology, cause, or causes, which are acute with an infection or chronic, of which the causes are numerous. Where a neuromodulatory implant systems might eliminate the need for drugs, much benefit is attained. Until establishing the cause and identifying drugs that will provide the same outcome without the need for an implanted system, the subject will remain academic. The mechanical voiding systems to be described not only eliminate the need for drugs but completely eliminate all of the symptoms that had prompted use of the drugs, to include incontinence, nocturia, and urge sensation however insistent or inopportune. Implanted to replace missing function for any of a number of reasons, the system functions as a prosthesis. As a mechanism the user can turn on or off as necessary, the system functions as a micturative or urinary assist device.

By directly delivering urine from the ureters into a collection bag or into an insensate synthetic neobladder whence transfer to the collection bag is automatic, voiding proceeds without the conscious awareness of the patient, who for all intents and purposes ceases to urinate. This condition is similar to continuous urinary incontinence due to the congenitally anomalous bypassing of the bladder by either or both ureters which then terminate instead in the urethra; however, instead of uncontrolled emission, the implant system institutes full control. The patient is aware of only the weight of the collection bag which is emptied into a proper receptacle through its outlet hose.

The patient ambulatory or not, such a totally implanted system allows the hygienic elimination of urine following evisceration to eradicate pelvic cancer or traumatic injury without the need for reconstructive surgery unlikely to prove completely dependable anyway. The ability to provide such a system in a form that will expand with growth means that the inability of a neonate to void urine without interference due to any of a large number of congenital urinogenital (genitourinary, urogenital) tract defects can be offset before, during, and after any stage in repair.

Providing an alternate route for the drainage of urine eliminates the need to provide and maintain such an alternate path mid- and postoperatively. The system remains in place not only to offset any deficits in urinary function which remain following any stage in repair but to directly pipeline medication to the relevant site or sites during or after any stage automatically. The defects relevant here are not those usually repaired to satisfaction such as ureters congenitally terminated improperly or mispositioned such as retrocavally, but rather defects which necessitate a staged series of operations warranting postoperatively continued medicinal support administered by a totally implanted automatic drug targeting system.

Such congenital conditions may involve multiple defects, necessitate bladder reconstruction that often leaves behind a residual refractory vesicoureteral reflux, bladder exstrophy with or without epispadias, penile or vaginal agenesis, and ambiguous genitalia as may present with the Klinefelter or Turner syndromes. In pediatric, mentally disturbed, and cognitively impaired patients, the automatic timely release of drugs without the need to depend upon the patient or an attendant for compliance is a significant advantage. Congenital defects that interfere with the ability to urinate normally include the agenesis or malformity of any structure or combination of structures along the urinary tract.

Interference in the free flow of urine so that an obstruction or restriction causes urine to pool leads to secondary problems such as crystal formation and infection (see Seifter, J. L. and Brenner, B. M. 2005. “Urinary Tract Obstruction,” in Kasper, D. L. Braunwald, E., Fauci, A. S., Hauser, S. L., Longo, D. L., and Jameson, J. L. Harrison's Principles of Internal Medicine, Chapter 270, pages 1722-1724, New York, New York: McGraw-Hill Medical Publishing Division). Accordingly, the object in a prosthetic system must be not simply to allow sanitary voiding but to maintain the system itself in an unobstructed and uninfected condition.

Most operations such as organ transplants and most disorders are best treated through the use a certain type of valve. For example, an adjustable urinary voidance control system such as that shown in FIG. 30 could substitute a solenoid-driven vascular valve or linear servomotor such as that used in FIG. 10A—shown in FIG. 9 to comprise cylindrical housing 37, enclosing adjacent circular electromagnet coils 38 in circumferential and surrounding relation to the motor shaft 39 which incorporates successive pairs of cylindrical neodymium iron boron permanent magnets 42—for the wearer manually operated push/pull, or Bowden, cable mechanism shown. However, in a mentally competent patient, such use would be uneconomic.

While a urinary assistance device or prosthesis is never to be implanted where conventional treatment would allow the avoidance thereof, once diversion jackets such as shown in FIG. 28 of servovalves such as shown in FIG. 30 have been placed, the accessory channels can be used to eradicate a urinary tract infection by running an antimicrobial such as hydrogen peroxide, benzalkonium chloride, chlorhexidine, or povidone-iodine into the ureters to coat the ureteral, bladder, and upon voiding, the urethral urothelium. Antiseptics that cause a stinging sensation should be preceded by lidocaine.

Thus, where the lower urinary tract is bypassed by a prosthetic voidance control system consisting entirely of synthetic materials such as that shown in FIG. 28 so that infection generates no experiential correlate as the sensation of irritation or pain, but poses the threat of serious sequelae such by retrograde migration to induce a pyelonephritis, pathogen intrusion, with the deposition of a biofilm is eradicated by periodically direct pipeline targeting an antimicrobial, allowing the avoidance of an antibiotic such as ciprofloxacin or vancomycin if bacterial (most often Escherichia coli), for example, through an accessory channel, which all ductus side-entry and inline coupling jacket tie-line mounted jackets, valves, and servovalves incorporate.

When urinary diversion is not required, the direct targeting of drugs or a stone solvent into a ureter can be accomplished through a basic ductus side-entry-jacket with accessory channel as shown in FIG. 16 in U.S. Pat. No. 11,759,186, entitled Ductus Side-entry Jackets and Prosthetic Disorder Response Systems and its parent application Ser. No. 14/121,365 which shows which depicts such a jacket connected to the left descending coronary artery. When dealing with a coronary or carotid artery, consideration should be given to the preliminary use of an anti-vasospasmic, or anti-spastic.

Basic ductus side-entry jackets very small and light in weight, in some rare circumstances, the diversion of blood into—not out of—such a crucial vessel can be from a vascular valve or servovalve on a large nearby artery into a basic side-entry jacket on the carotid or coronary. Due to increased size and weight and the forcefulness of systolic contraction, vascular servovalves are not used on a coronary artery, instead, a nonjacketing side-entry connector as described in U.S. Pat. No. 11,013,858, entitled Nonjacketing Side-entry Connectors and Prosthetic Disorder Response Systems, can be positioned over the supply field of the coronary to directly deliver the drug into the myocardium.

Such an automatic system is also appropriate for use in a patient with urinary tract sensation but mentally impaired, disturbed, or presenting with irreversible urinary incontinence. By contrast, intractable nocturia is best alleviated through the use of a urinary assistance system such as shown in FIG. 30 rather than the prosthesis shown in FIG. 28. This direct route of administration renders superfluous the route of infection whether through ingestion, inhalation, breaches in the skin caused by ectoparasites, such as arthropod, or vertebrate bites, or sexual activity. Drugs delivered thus are not injected intramuscularly to become dispersed throughout the circulation but rather infused into the immediate blood supply as to bypass nontargeted tissue.

For this reason, direct pipeline targeting allows the use of dose levels which due to adverse side effects, dispersion throughout the systemic circulation would preclude. Provided an anti-pathogenic agent is available, the agent of disease when bacterial, mycotic, protozoan, or helminthic makes little difference. The wearer of a urine voiding prosthesis and assistance device such as that shown in FIG. 30 retains normal sensation and is therefore able to control voiding as well as the administration of drugs. A urinary tract infection then can be treated through the oral route with ciprofloxacin, for example. These considerations as to drug administration pertain no less to the use of crystal solvents in stone formers.

The temporary unilateral or bilateral diversion of urine from a ureter or both ureters to a paracorporeal collection bag can facilitate healing of the bladder and lower urinary tract with drugs following surgery. The diversion jacket incorporates at least one accessory channel for the delivery of drugs directly into the lumen of the segment to be treated. For patients with irreversible neurogenic bladder dysfunction, agenesis (aplasia) of or irreparable trauma to the urinary tract, radical cystectomy, bladder exstrophy (ectopia vesicae), or a pelvic exenteration (evisceration), use is continual and permanent, supplanting the need for an ileal conduit or a neobladder, which if lacking the necessary volume, compels frequent urination.

A central problem in urological practice is that the end-symptoms of numerous different and sometimes overlapping causes of urinary dysfunction may affect the patient in numerous combinations to various degrees while remaining scarcely if at all distinguishable, making more than a general differential diagnosis difficult if not impossible. Electrostimulation and a mechanical collection system have the advantage of helping the patient with an overactive bladder and/or incontinence that is diagnosis independent, that is, regardless of the cause or combination of causes according to a complex diagnosis (see, for example,

Zomkowski, K. Kammers, I. Back B. B. H., Moreira, G. M., Sonza, A., Sacomori, C., and Sperandio, F. F. 2022. “The Effectiveness of Different Electrical Nerve Stimulation Protocols for Treating Adults with Non-neurogenic Overactive Bladder: A Systematic Review and Meta-analysis,” International Urogynecology Journal preprint PMID [PubMed Identifier]: 35119495; Tayyeb, M. and Tadi, P. 2021. “Neurogenic Bladder,” Treasure Island, Florida: StatPearls Publishing Co.; PeyTonnet, B., Mironska, E. Chapple, C., Cardozo, L., Oelke, M., and 6 others 2019. “A Comprehensive Review of Overactive Bladder Pathophysiology: On the Way to Tailored Treatment,” European Urology 75(6): 988-1000; Gormley, E. A., Lightner, D. J., Faraday, M. and Vasavada, S. P. 2015. “Female Pelvic Medicine. Diagnosis and Treatment of Overactive Bladder (Non-neurogenic) in Adults,” Journal of Urology (Philadelphia, Pennsylvania) 193(5): 1572-1580; Dorsher, P. T. and McIntosh, P. M. 2012. “Neurogenic Bladder,” Online, Advances in Urology (Cairo, Egypt) 2012:816274).

Gut is not adapted to convey urine, and when made to do so, gradually undergoes metaplastic transition that can result in malignancy and the need for resection. Should this occur, the placement of a prosthesis made of synthetic materials can prevent further disease and the need for revision. This is far less traumatizing or prone to complications than is the harvesting (retrieving, recovering, procurement) of gut, or the prospect of a radical cystectomy if not exenteration to eradicate cancer or to end unrelenting pain from interstitial cystitis (since redesignated chronic bladder pain syndrome).

For these reasons, the urothelium is usually protected following the creation of a urinary conduit from gut through the placement of a urinary diversion stent, and the ureter or ureters protected with J-stents. Special stents are now available for lining the ileal conduit to avert metaplastic degeneration; however, not all of the gut lining, or mucosa, is likely to be protected thus, and frequent if not constant contact with either urine or the stent with the lining of the gut can be irritating and result in erosive lesioning.

Conventional stents separate the urine from the urothelium but lack any means for maintaining the patency of the synthetic lumen such as through the direct targeting into the synthetic lumen of the interface between the outer surface of the stent and the urothelium of occlusion counteractants. Repeated exposure to urine without intermittent rinsing with clean water or an antiseptic solution fouls a catheter or stent in itself, to which must be added the gradual accretion of crystal if not a biofilm making these devices short-lived and requiring the unpleasant inconvenience of replacement at relatively frequent intervals, typically, once a month.

Such represents not a solution but the perpetuation of nuisance. In marked contrast, the means described herein for replacement of portions if not the entire lower urinary tract are cleaned and as necessary decrystallized automatically by an implanted microcontroller which signals the pump at the outlet of the antiseptic and/or decrystallizing solution drug reservoir to release the amount into the prosthesis as prescribed in its prescription-program. Provided the reservoir is replenished, the patient can remain as oblivious to the condition as had the urinary tract remained normal.

Where side-entry jackets can divert or return part of the flow through a ductus such as a blood vessel or ureter, a side-entry diversion jacket can divert all or any fraction of the flow. If the carotid diversion device to be described is to remain in place indefinitely as a prosthesis, a subdermally implanted port is placed to allow the periodic injection of an anticoagulant for direct targeting to the treatment site at the carotid bulb or bifurcation.

The significance of using valved rather than unvalved or basic ductus side-entry jackets along the urinary tract is that unvalved jackets are static, while valved jackets are controllable, allowing the flow of urine to be switched from the bladder during the day to an external collection bag during the night, for example. Flow through the valve mainline is controlled by adjustment of the diversion chute by the implanted microcontroller, which to control the delivery of drugs through the accessory channel or channels adjusts the drug reservoir outlet aperture or pump. In a ureteral takeoff side-entry jacket, the primary passageway or mainline is used to divert urine, and one or more sidelines, or accessory channels are used to allow the targeted delivery of drugs into the jacket and substrate ureter directly from a small port at the body surface.

For example, by placing a nonjacketing side-entry connector as described in U.S. Pat. No. 11,013,858 to drain the renal pelvis and another on the bladder dome, the two can be connected with a catheter kept from fouling by the direct delivery through the accessory channel of the upper connector of suitable agents fed from a body surface port. The considerable advantages in the directly targeted delivery of drugs in an effective dose to primary and secondary sites of disease rather than its dispersal throughout the circulation where unintended tissue is exposed so that the dose must be minimized and still pose the risk of long known adverse side effects, drug-drug, and drug-food interactions (see, for example, Sanjay, S. T., Dou, M., Fu, G., Xu, F., and Li, X. 2016. “Controlled Drug Delivery Using Microdevices,” Current Pharmaceutical Biotechnology 17(9): 772-787).

Given multiple capabilities, to include the diversion of flow, direct delivery to lesions of medication, and the ability to incorporate sensors, side-entry jackets, diversion valves, and nonjacketing side-entry connectors can join and diagnose native tissue, and maintain the patency of prosthetic replacement parts in the lower urinary tract, for example. A nonprosthetic urinary assist system allows the patient to sleep without interruption due to urge sensation and to appear in public even though affected by urinary incontinence or nocturia, for example, not caused by an infection such as a ureteritis, cystourethritis, or pyeloureteritis dispelled by a ten-day course of an oral antibiotic, for example.

Conditions demanding surgery such as a hemorrhage (see, for example, Thompson, J. S. and McAlister, W. H. 1975. “Subepithelial Hemorrhage in the Renal Pelvis and Ureter Simulating Pyeloureteritis Cystica,” Pediatric Radiology 3(3): 156-157), to include those requiring excision such as ureteral endometriosis, can usually be repaired endoscopically, ductus side-entry and side- entry diversion jackets incorporating the required components thereafter used to expedite healing and protect against infection (see, for example, Babu, S. M., Sandeep, P., Pathade, A., and Nagaraj, H. K. 2014. “Bleeding Ureter: Endometriosis Mascarading [sic-Masquerading] as a Ureteral Malignancy-A Case Report,” Journal of Clinical and Diagnostic Research 8(10): ND10-ND11; Wasserman, N. F. 1996. “Inflammatory Disease of the Ureter,” Radiologie Clinics of North America 34(6): 1131-1156; McComb, R. A. 1930. Diseases and Anomalies of the Ureters,” Canadian Medical Association Journal 22(4): 484-488).

Temporary diversion from the distal urinary tract of irritating if not endogenously or exogenously contaminated or toxic urine, sometimes as a consequence of a disturbance in kidney function, expedites healing of diseased or operated upon tissue of the tract below that level, to which the diversion jacket can also target medication, and avert serious complications, such as interstitial cystitis (since redesignated chronic bladder pain syndrome)(see, for example, Argade, S., Berecz, Z., Su, Y., and Parsons, C. L. 2016. “Increased Toxic Urinary Cations in Males with Interstitial Cystitis: A Possible Cause of Bladder Symptoms,” World Journal of Urology 34(12): 1685-1691; Golabek, T., Bukowczan, J., Sobczynski, R., Leszczyszyn, J., and Chlosta, P. L. 2016. “The Role of Micronutrients in the Risk of Urinary Tract Cancer,” Archives of Medical Science 12(2): 436-447; Golabek, T., Powroźnik, J., Chłosta, P., Dobruch, J., and Borówka, A. 2015. “The Impact of Nutrition in Urogenital Cancers,” Archives of Medical Science 11(2): 411-418; Parsons, C. L., Shaw, T., Berecz, Z., Su, Y., Zupkas, P., and Argade, S. 2014. “Role of Urinary Cations in the Aetiology of Bladder Symptoms and Interstitial Cystitis,” BJU [British Journal of Urology] International 114(2): 286-293; Inoue, Y., Umezaki, M., Jiang, H., Li, D., Du, J., Jin, Y., and 4 others 2014. “Urinary Concentrations of Toxic and Essential Trace Elements Among Rural Residents in Hainan Island, China,” International Journal of Environmental Research and Public Health 11(12): 13047-13064; Birgisdottir, B. E., Knutsen, H. K., Haugen, M., Gjelstad, I. M., Jenssen, M. T., and 5 others 2013. “Essential and Toxic Element Concentrations in Blood and Urine and Their Associations with Diet: Results from a Norwegian Population Study Including High-consumers of Seafood and Game,” Science of the Total Environment 463-464:836-844; inci, M., Zararsιz, i., Davarcι, M., and Gorur, S. 2013. “Toxic Effects of Formaldehyde on the Urinary System,” Turkish Journal of Urology 39(1): 48-52; Lin, C. N., Wang, L. H., and Shen, K. H. 2009. “Determining Urinary Trace Elements (Cu, Zn, Pb, As, and Se) in Patients with Bladder Cancer,” Journal of Clinical Laboratory Analysis 23(3): 192-195; Rajasekaran, M., Stein, P., and Parsons, C. L. 2006. “Toxic Factors in Human Urine that Injures Urothelium,” International Journal of Urology 13(4): 409-414).

Where bladder pain syndrome or interstitial cystitis (since redesignated chronic bladder pain syndrome) is already present, medication directly delivered into the lower urinary tract in addition to pentosan polysulfate, sodium can include such agents as dimethyl sulfoxide, heparin, lidocaine, botulinum neurotoxin A, oxychlorosene sodium, liposomal phospholipid vesicles, and stem cells, usually in various combinations (Cvach, K. and Rosamilia, A. 2015. “Review of Intravesical Therapies for Bladder Pain Syndrome/Interstitial Cystitis,” Translational Andrology and Urology 4(6): 629-637; Kim, A., Shin, D. M., and Choo, M. S. 2016. “Stem Cell Therapy for Interstitial Cystitis/Bladder Pain Syndrome,” Current Urology Reports 17(1): 1; Colaco, M. A. and Evans, R. J. 2013. “Current Recommendations for Bladder Instillation Therapy in the Treatment of Interstitial Cystitis/Bladder Pain Syndrome,” Current Urology Reports 14(5): 442-447; see also, for example, Chiu, B., Tai, H. C., Chung, S. D., and Birder, L. A. 2016. “Botulinum Toxin A for Bladder Pain Syndrome/Interstitial Cystitis,” Toxins (Basel) 8(7). pii: E201).

To treat superficial lesions of the urothelial lining of the urinary tract or the endothelium lining a blood vessel, the electromagnet jacket is adjusted to project a lower field strength. To treat a deeper lesion, the field strength is increased. If the former is a carcinoma and/or the latter a sarcoma, a jacket with a radiation shielding outer coating is used to allow the direct targeting of a radioactive agent such as a nanoparticulate radionuclide, or superparamagnetic iron oxide nanoparticles, to the treatment site (Sahdev, P., Ochyl, L. J., and Moon, J. J. 2014, Op cit.). Because irradiating the epithelial lining of a ductus is no less capable of initiating a metaplastic cascade culminating in frank carcinoma, such use warrants close evaluation as to the extent of malignancy.

Magnetized side-entry and diversion jackets overcome a central problem in the lack of an ability to directly target magnetically carrier-bound (carrier-bonded), hence magnetically susceptible rendered stem cells to the diseased or lesioned tissue (“A fundamental impediment for the evolution of stem cell therapies has been the difficulty of effectively targeting administered stem cells to the disease foci.”-Cores, J., Caranasos, T. G., and Cheng, K. 2015. “Magnetically Targeted Stem Cell Delivery for Regenerative Medicine,” Journal of Functional Biomaterials 6(3): 526-546).

In rare instances of metastasis from the prostate to the ureters, the direct targeting of radioisotopes to the lesions may detain if not eliminate the need for a ureterectomy (see, for example, Zhang, D., Li, H., and Gan, W. 2016. “Hydronephrosis Associated with Ureteral Metastasis of Prostate Cancer: A Rare Case Report,” Molecular and Clinical Oncology 4(4): 597-598; Vartak. A. and Sucheck, S. J. 2016. “Recent Advances in Subunit Vaccine Carriers,” Online, MDPI [Multidisciplinary Digital Publishing Institute] Vaccines (Basel, Switzerland) 4(2) pii: E12; Morales, I., Bassa, C., Pavlovic. A., and Morales, C. 2015. “Ureteral Metastasis Secondary to Prostate Cancer: A Case Report,” Urology Case Reports 5:4-5).

If urine is an irritant for the diseased segment, irritant counter active agents are among those used. For example, acidity as an irritant is counteracted by including sodium bicarbonate. Body surface ports positioned on the outside of the skin are referred to herein as cutaneous, while those positioned subdermally are referred to as subdermal, subcutaneous, or hypodermic. Except where the condition treated is terminal, central lines and portacath catheters are seldom intended for lifetime use without replacement. In contrast, a central line with distal end connected by a ductus side-entry or diversion jacket to the target ductus, typically a major vein in the upper chest, is fixed in position as not to limit the activities in which the patient will be free to participate.

Along the urinary tract, the small body surface or extracutaneous port—about 1.0 centimeter in outer diameter with openings for accessory channels, button cell battery, control knobs, and push/pull control knobs. 1.0 centimeter with only a battery added to the effluent mainline-much smaller than and not unsightly as is a stoma. In an adult, a stoma is well over an inch, or 25.4 millimeters, in every dimension, except for a period of weeks after its creation, when it remains quite swollen. Unlike a stoma, the small plastic port allows dispensing with repeated endoscopic treatment, and in some cases, stenting, both of which risk additional complications and the need for drugs risking additional complications.

In a ureter, the avoidance of repeated endoscopy can avert strictures or the aggravation of strictures as a consequence of stimulating spongiofibrosis (see, for example, Zhang, Y. M., Chu, P., and Wang. W. J. 2017. “PRISMA [Preferred Reporting Items for Systematic Reviews and Meta-Analyses]-combined α-blockers and Antimuscarinics for Ureteral Stent-related Symptoms: A Meta-analysis,” Medicine (Baltimore, Maryland) 6(7): e6098; Das, K., Ordones, F., Welikumbura, S., and Brook, N. R. 2016. “Ureteroiliac Artery Fistula Caused by a Metallic Memokath Ureteral Stent in a Radiation-Induced Ureteral Stricture,” Journal of Endourology Case Reports 2(1): 162-165; Yan, H., Wang. Y., Sun, R., and Cui. Y. 2016. “The Efficacy of Antimuscarinics Alone or in Combination with Alpha-Blockers for the Treatment of Ureteral Stent-Related Symptoms: A Systematic Review and Meta-Analysis,” Urologia Internationalis October 20 (in press); Koprowski, C., Kim, C., Modi, P. K., and Elsamra, S. E. 2016. “Ureteral Stent-associated Pain: A Review,” Journal of Endourology 30(7): 744-753; Maldonado-Avila, M., Garduno-Arteaga, L., Jungfermann- Guzman, R., Manzanilla-Garcia, H. A., Rosas-Nava, E. and 4 others 2016. “Efficacy of Tamsulosin, Oxybutynin, and Their Combination in the Control of Double-J Stent-related Lower Urinary Tract Symptoms,” International Brazilian Journal of Urology 42(3): 487-493; Song, G., Lim, B., Han, K. S., Song, S. H., Park, H. K., and Hong. B. 2015. “Complications after Polymeric and Metallic Ureteral Stent Placements Including Three Types of Fistula,” Journal of Endourology 29(4): 485-489; Siorek, M., Tierney, B., Fowler, J., and Dowell, J. D. 2015. “Ureteroarterial Fistula Following Retrograde Ureteral Stenting in a Patient with a Double-barreled Wet Colostomy for Cervical Cancer,” Gynecologie Oncology Reports 13:44-46; Chung, J. M., Park, C. S., and Lee, S. D. 2015. “Postoperative Ureteral Obstruction after Endoscopic Treatment for Vesicoureteral Reflux,” Korean Journal of Urology 56(7): 533-539; Wong. E., Tse. V., and Wong, J. 2014. “Durability of Memokath™ Urethral Stent for Stabilization of Recurrent Bulbar Urethral Strictures-Medium-term Results,” BJU [British Journal of Urology] International 113 Supplement 2:35-39; Miyaoka, R. and Monga. M. 2009. “Ureteral Stent Discomfort: Etiology and Management,” Indian Journal of Urology 25(4): 455-460).

In a surface port that includes an effluent opening such the port shown in FIG. 26 with urine outlet, the protrusion through the skin of this opening assists in fixing the port in position. Including openings for the accessory channels in the same extracutaneous port allows reduction in the outer diameter of the port, the injection or infusion entry points clearly seen. To make it smaller in diameter, hence, less noticeable, a surface port with an effluent opening in lieu of a urinary stoma can be provided with subdermal, that is, subsurface, accessory channel entry holes.

When placed to a side of the mons pubis, the port is permanently out of sight, so that this consideration does not apply. Unless the esthetic factor is more important to the patient who will self-administer any necessary medication at home, this is not preferred. When the port does not include an effluent or other excurrent opening, it is placed subdermally or subcutaneously, where it is out of view. A port for placement thus is made larger in outer diameter—about 2.0 centimeters—with entry holes likewise larger.

Even though lodged within subcutaneous fat, this reduces any potential for the port to shift in position and the risk of misaligning the injection or infusion needle or fine cannula to the correct entry hole. The accuracy of injection or infusion is improved by identifying subdermal openings with a small dot, or number or letter identifier on the overlying skin. If the device is removed, these markings can be removed by laser. The strong shallow dome cover optimally disperses an impact from any angle to provide the maximum protection for a device which must have some protrusion from the skin. In contrast, a stoma, whether end or loop, is exposed, vulnerable, and will cause much disruption, and possibly, and if significantly traumatized, the need for revision.

Tiny (millimetric) in size, not weakening the abdominal muscles, the port is not dependent upon tissue adhesion and therefore not subject to skin irritation dehiscence, or structural failure in the form of prolapse, retraction, or herniation, and if stuck, is unlikely to result in injury comparable in consequence to exposed gut or gut enclosed within material sheeting used to aid cleanliness and prevent odor. Upon healing, the port is easily removed. Ureteral stents become encrusted, requiring replacement, which the ability to directly drip a stone solvent through the accessory channel of a ductus side-entry jacket positioned at a higher level would eliminate. The small size, negligible if any protrusion as seen, and invulnerability to injury compared to a stoma liberalizes its positioning along the body surface.

For example, with miniature pump assistance, a port placed in the mons pubis can directly target medication through their respective accessory channels to one or more side-entry connectors and/or jackets in different locations cephalad, or craniad, thereto. The area previously cleared by electrolysis, a mons pubis port is easily seen for self-injection, especially by the elderly and other infirm with comorbidities requiring several port accessory channel openings. If necessary pending the completion of electrolysis, the port can remain loose enough as not to interfere with completion of the electrolytic process. A urine outlet requiring a cutaneous or excurrent opening in any event, viewability is further enhanced by making the openings for the accessory channels likewise cutaneous rather than subcutaneous.

Areas where a stoma cannot be positioned because it would interfere with clothing become available. Relative freedom in port positioning also allows greater flexibility in avoiding less desirable routes for the lines connected to the port internally. In some instances, this allows finding a less tortuous path, allowing gravity to eliminate the need for assist pumping to drive the injectant to the target tissue. Relative freedom at line routing also reduces the risk of strangulation or entrapment. The risk of discomfort due to the encroachment upon neighboring tissue by electrical control lines even when strategically routed is provided by blue tooth remote control from the microcontroller.

The direct targeting of drugs through an accessory channel in the surface port to the site under treatment allows the use of smaller doses less frequently, which can be increased in concentration, the avoidance of untargeted tissue averting many if not all of the usual adverse side, drug-drug, and drug-food side effects. The addition of a basic side-entry jacket on the venous outflow allows the release of a reversal agent, or counteractant, to remove even trace amounts from the circulation. Except to allow direct viewing, relatively few procedures currently accomplished through endoscopy, and those mostly in the gastrointestinal rather than the urinary tract, such as bulking of the lower esophageal sphincter to prevent gastroesophageal reflux, cannot be supplanted by the means described.

Moreover, vesicoureteral reflux can be suppressed with an antacid drip readily established with these means, and a fine fiberscope can be passed through any opening in the surface port. An accessory channel can be used in lieu of endoscopy to coat the internal wall with contrast and to deliver a stone solvent, averting the need for and the complications associated with endoscopy, to include ureteral avulsion, perforation, and mucosal abrasion (see, for example, Tanimoto, R., Cleary, R. C., Bagley, D. H., and Hubosky, S. G. 2016. “Ureteral Avulsion Associated with Ureteroscopy: Insights from the MAUDE [Manufacturer and User Facility Device Experience] Database,” Journal of Endourology 30(3): 257-261; Tang, K., Sun, F., Tian, Y., and Zhao, Y. 2016. “Management of Full-length Complete Ureteral Avulsion,” International Brazilian Journal of Urology 42(1): 160-164; Chung, J. M., Park, C. S., and Lee, S. D. 2015. “Postoperative Ureteral Obstruction after Endoscopic Treatment for Vesicoureteral Reflux,” Korean Journal of Urology 56(7): 533-539; Hubosky, S. G., Raval, A. J., and Bagley, D. H. 2015. “Locked Deflection During Flexible Ureteroscopy: Incidence and Elucidation of the Mechanism of an Underreported Complication,” Journal of Endourology 29(8): 907-912; Gaizauskas, A., Markevicius, M., Gaizauskas, S., and Zelvys, A. 2014. “Possible Complications of Ureteroscopy in Modern Endourological Era: Two-point or “Scabbard” Avulsion,” Case Reports in Urology 2014:308093; Burks, F. N. and Santucci, R. A. 2014. “Management of latrogenic Ureteral Injury,” Therapeutic Advances in Urology 6(3): 115-124; Gao, P., Zhu, J., Zhou, Y., and Shan, Y. 2013. “Full-length Ureteral Avulsion Caused by Ureteroscopy: Report of One Case Cured by Pyeloureterostomy, Greater Omentum Investment, and Ureterovesical Anastomosis,” Urolithiasis 41(2): 183-186).

The accessory channel can be used to deliver medication such as a corticosteroid to avert inflammation and or lidocaine, for example, to reduce pain that would necessitate stenting with its complications, which include migration, infection, fragmentation, residual fragmentation following removal, vesicoureteral reflux, encrustation, ureteroarterial fistulization, necrosis, dysuria, hematuria, frequent urination, pain, and usually require removal of the stent (e1 Khader, K. 1996. “Complications of Double J Ureteral Stents,” (in French with English abstract at Pubmed) Journal d'urologie (Paris) 102(4): 173-175)(see also, for example, Gu, Y., Zhang, J., and Wang, G. 2016. “Use of Cystourethroscopy to Remove an Indwelling Double-J Ureteral Stent 6 years Following Simultaneous Radical Sigmoid Colon Cancer and Partial Bladder Resection: A Case Report,” Experimental and Therapeutic Medicine 11(6): 2467-2469; Hikmet, T., Sercan, S., Ugur, O. H., Burhan, B., and 4 others 2015. “A New Method for Fragmented Ureteral Stent Extraction: Flexible Renoscopy,” Urology Case Reports 3(6): 190-192; Pais, V. M. Jr., Chew, B., Shaw, O., Hyams, E. S., Matlaga, B., Venkatesh, R., Page, J., and 4 others 2014. “Percutaneous Nephrolithotomy for Removal of Encrusted Ureteral Stents: A Multicenter Study,” Journal of Endourology 28(10): 1188- 1191; Ahallal, Y., Khallouk, A., El Fassi, M. J., and Farih, M. H. 2010. “Risk Factor Analysis and Management of Ureteral Double-J Stent Complications,” Reviews in Urology 12(2-3): e147-e151; Ku, J. H., Lee, S. W., Jeon, H. G., Kim, H. H., and Oh, S. 2004. “Percutaneous Nephrostomy Versus Indwelling Ureteral Stents in the Management of Extrinsic Ureteral Obstruction in Advanced Malignancies: Are There Differences?,” Urology 64(5): 895-899; Spirnak, J. P. and Resnick, M. I. 1985. “Stone Formation as a Complication of Indwelling Ureteral Stents: A Report of 5 Cases,” Journal of Urology (Philadelphia, Pennsylvania) 134(2): 349-351). Additional references pertaining to the complications associated with ureteral stents are cited below.

When catheters were placed to convey urine on a long-term basis, it soon became clear that these would inevitably become fouled and eventually occluded by a buildup of infectious matter and/or crystalline stone, and no counteractant could be systemically circulated at the high dose that would be required to overcome the resistance of biofilm-forming bacteria or prevent the buildup of mineral encrustation. Early experience at fully implanting catheters to carry sterile blood became occluded with thrombus, and those placed to convey urine were soon found to develop a biofilm, sometimes with a crystalline encrustation (see, for example, Verma, A., Bhani, D., Tomar, V., Bachhiwal, R., and Yadav, S. 2016. “Differences in Bacterial Colonization and Biofilm Formation Property of Uropathogens between the Two most Commonly used Indwelling Urinary Catheters,” Journal of Clinical and Diagnostic Research 10(6) PC01-PC03; VanEpps, J. S. and Younger, J. G. 2016. “Implantable Device-related Infection,” Shock (Augusta, Georgia) 46(6): 597-608; Delcaru, C., Alexandru, I., Podgoreanu, P., Grosu, M., Stavropoulos, E., Chifiriuc, M. C., and Lazar, V. 2016. “Microbial Biofilms in Urinary Tract Infections and Prostatitis: Etiology, Pathogenicity, and Combating Strategies,” Pathogens (Basel) 5(4) pii [publisher item identifier]: E65; Murugan, K., Selvanayaki, K., and Al-Sohaibani, S. 2016. “Urinary Catheter Indwelling Clinical Pathogen Biofilm Formation, Exopolysaccharide Characterization and their Growth Influencing Parameters,” Saudi Journal of Biological Sciences 23(1): 150-159; Melo, L. D., Veiga, P., Cerca, N., Kropinski, A. M., Almeida, C., Azeredo, J., and Sillankorva, S. 2016. Development of a Phage Cocktail to Control Proteus mirabilis Catheter-associated Urinary Tract Infections,” Online, Frontiers in Microbiology 7:1024; Feneley, R. C., Hopley, I. B., and Wells, P. N. 2015. “Urinary Catheters: History, Current Status, Adverse Events and Research Agenda,” Journal of Medical Engineering and Technology 39(8): 459-470; Lehman, S. M. and Donlan, R. M. 2015. “Bacteriophage-mediated Control of a Two-species Biofilm Formed by Microorganisms Causing Catheter-associated Urinary Tract Infections in an In Vitro Urinary Catheter Model,” Antimicrobial Agents and Chemotherapy 59(2): 1127-1137; Schaffer, J. N. and Pearson, M. M. 2015. “Proteus mirabilis and Urinary Tract Infections,” Microbiology Spectrum 3(5); Nzakizwanayo, J., Hanin, A., Alves, D. R., Mccutcheon, B., Dedi, C., and 9 others 2015. “Bacteriophage Can Prevent Encrustation and Blockage of Urinary Catheters by Proteus mirabilis,” Antimicrobial Agents and Chemotherapy 60(3): 1530-1536; Stickler, D. J. 2014. “Clinical Complications of Urinary Catheters Caused by Crystalline Biofilms: Something Needs to be Done,” Journal of Internal Medicine 276(2): 120-129; Guggenbichler, J. P., Assadian, O., Boeswald, M., and Kramer, A. 2011. “Incidence and Clinical Implication of Nosocomial Infections Associated with Implantable Biomaterials-Catheters, Ventilator-associated Pneumonia, Urinary Tract Infections,” GMS [German Medical Science] Krankenhaushygiene interdisziplinar [interdisciplinary hospital hygiene] 6(1): Document 18; Jacobsen, S. M. and Shirtliff, M. E. 2011. “Proteus mirabilis Biofilms and Catheter-associated Urinary Tract Infections,” Virulence 2(5): 460-465; Wyndaele, J. J. 2010. “The Encrustation and Blockage of Long-term Indwelling Catheters,” Spinal Cord 48(11): 783; Stickler, D. J. and Feneley, R. C. 2010. “The Encrustation and Blockage of Long-term Indwelling Bladder Catheters: A Way Forward in Prevention and Control,” Spinal Cord 48(11): 784-790; Broomfield, R. J., Morgan, S. D., Khan, A., and Stickler, D. J. 2009. “Crystalline Bacterial Biofilm Formation on Urinary Catheters by Urease-producing Urinary Tract Pathogens: A Simple Method of Control,” Journal of Medical Microbiology 58 (Part 10): 1367-1375).

TABLE 2
Table 2. Appurtenants of adjustable assist device and
nonadjustable prosthesis urinary diversion components.

| | NONADJUSTABLE | | ADJUSTABLE | |
|---|---|---|---|---|
| COMPONENTS | TEMPORARY | PROSTHESIS | DAYTIME | NIGHTTIME |
| Cables | No | No | Yes | Yes |
| Chute control nobs | No | No | Yes | Yes |
| Ureteral end-plugs | No | Yes | No | No |
| Jacket outer obturator | No | No | Yes | Yes |
| Ejection assist device | Yes | Yes | No | Yes |
| Ejection assist battery | Yes | Yes | No | Yes |

The table above shows the different components required in each of the four embodiment types. Ureteral takeoff diversion jackets fall into two categories, one nonadjustable, comprising two embodiments, one for temporary use to expedite healing of the lower tract, the other for permanent use as a prosthesis when the lower tract is missing or dysfunctional, the other category adjustable. Adjustable embodiments are either for daytime use when the wearer is erect but unable to use a bathroom, or for use while the wearer is asleep.

With all four embodiments, diversion is to an external collection bag, requiring an abdominal or pelvic surface outflow port having features that make these less likely than a stoma to create skin problems. These include a removable cover that when removed exposes openings through which skin irritant counteractants can be instilled or injected. Takeoff at the ureter or ureters has the advantage of proceeding without the voluntary participation of the wearer. External urinary drainage devices such as single-use (Texas, urisheath/uriliner, “condom catheter,” diver's drysuit p-valve catheter) do not allow this. For daytime use, there is, therefore, no distraction from or need to interrupt a public performance.

Furthermore, the incorporation of suitable self-contained urinalysis biosensor microprobes, microprobes connected to a separately implanted microfluidic urinalysis laboratory on a chip, and/or pressure sensors in the diversion jacket and/or ductus side-entry jackets positioned at different levels along the urinary tract allow the automatic continuous collection of time-stamped diagnostic data which can be transmitted to a clinic without the need for the patient to go to the clinic in order to provide a urine sample of no more than instant, or point of care, significance (see, for example, Courbet. A., Renard, E., and Molina, F. 2016. “Bringing Next-generation Diagnostics to the Clinic Through Synthetic Biology,” EMBO [European Molecular Biology Organization] Molecular Medicine 8(9): 987-991: Cobo, A., Sheybani, R., and Meng, E. 2015. “MEMS [microelectromechanical systems]: Enabled Drug Delivery Systems,” Advanced Healthcare Materials 4(7): 969-982; Sin, M. L., Mach, K. E., Wong, P. K., and Liao, J. C. 2014. “Advances and Challenges in Biosensor-based Diagnosis of Infectious Diseases,” Expert Review of Molecular Diagnostics 14(2): 225-244; Kim, S. and Roy, S. 2013. “Microelectromechanical Systems and Nephrology: The Next Frontier in Renal Replacement Technology,” Advances in Chronic Kidney Disease 20(6): 516-535).

With a prosthesis, when the dysfunction or agenesis is restricted to an intermediate portion of the urinary tract so that the urethra remains intact, diversion can be directly to the bladder, allowing voiding in a normal manner without the need for an external collection bag, the means therefor described and illustrated in U.S. Pat. No. 11,013,858. When diversion is to a neurogenic bladder lacking in urge sensation and/or voluntary contractibility, a pressure sensor is used to actuate a tiny, fully implanted, electric motor having a rotor driving an eccentric load, which signals the wearer of the need to void by vibrating. When a synthetic continent neobladder is used, takeoff is from the ureteral stump or stumps, which are connected to the neobladder by a nonjacketing side-entry connector.

If urination is frequent, a port with two button cell battery compartments is used to power the evacuation impeller and the synthetic neobladder stopper flap used to attain continence. The same device signals the need to void through a mons pubis port in a relatively normal manner. The wearer then initiates micturition by pressing a switch to obtain contraction through electrostimulation of the detrusor, either or both urethral sphincters, or all three. These alternatives to passive drainage into an external collection bag require supplementary electronics addressed in U.S. Pat. Nos. 11,013,858 and 11,759,186. U.S. Pat. No. 11,759,186 described ductus side-entry jackets, to include such details as the use of spring-loaded hinges to snap the jacket about the substrate ductus when placed.by the operator.

Referring to FIG. 28, for a patient who has been eviscerated or otherwise unlikely to recover continence, the device shown would be a fixed prosthesis in which parts 143 and 143′ were monolithic flow-diverting side-entry jackets with diversion chutes that are integrally molded. In a patient with kidney agenesis or one remaining kidney, the prosthesis would be unilateral from the outset. In a patient with urinary dysfunction and the prospect of full recovery, parts 143 and 143′ are diversion valves. Should the patient die, the device can be removed, sterilized in an autoclave or with ethylene oxide gas, and stored for placement in another patient. Since a temporary embodiment may have been placed to divert urine away from the lower tract to include the bladder in order to expedite healing, eventual removal will depend upon the outcome of the operation.

This is accomplished by chilling the obturator with a cold air gun. Because it must remain in constant use, the nonadjustable embodiment for use as a prosthesis omits miniaturized push/pull control cables and control knobs at the surface port used to advance and retract the side-entry jacket diversion chutes needed for elective use. The diversion chute is integral with an outlet obturator at its front or distal tip to close off the ureteral lumen when the chute is retracted. A central design consideration is the avoidance of all but the least urothelial compression that in both adjustable and nonadjustable embodiments will suffice to prevent leaks past the diversion chute when deployed, and in an adjustable embodiment, will suffice to prevent leaks through the side-entry hole as diversion outlet when the chute is retracted.

The significance of avoiding all but essential compression of the urothelium and suburothelium is addressed below. Diversion directly to the urethra so that the sensation of urgency is lacking necessitates the interpositioning of a pressure sensor in the diversion line to actuate a vibration motor, signaling the patient of the need to void, something not required with a neurologically functional bladder. The prosthesis does, however, require an evacuation assist device should the patient, especially a patient with impaired ureteric peristaltic function, adopt a posture as leaves gravity alone unable to achieve expulsion. The back pressure, not sensed by the wearer whose bladder is bypassed, then reaches the level where vesicoureteral reflux (regurgitation, backwash) of urine back up into the ureters results, boding serious complications.

Vesicoureteral or ureteral reflux in a neonate or infant can lead to hydroureteronephrosis and/or hydronephrosis, which if not treated promptly, will eventually result in end-stage renal failure. In a neonate or infant, the moisture barrier-protected viscoelastic polyurethane foam lining of the diversion jacket is made thick enough to allow the device to remain in place for the first several years. However, while the diversion jacket might last for life, if placed in early childhood, the diameter of the ureteral lumen will likely exceed the range of diameters the chute can prevent from leaking past it. With a young patient, the chute is therefore made wider and thinner to allow increased width with increased caliber or internal diameter of the ureteric lumen.

In an adult, the same diametrical compliance conforms to the significant variability in diameter of the ureter along its length. Adjustable embodiments are of two kinds, one for daytime use by a wearer who has normal urinary function but is unable to use a bathroom, and one for night-time use, when voiding will be with the wearer other than erect. There are other possibilities, such as a daytime wearer with impaired ureteral peristalsis who may require using the night-time embodiment. The causes of impaired ureteral peristaltic function are numerous, and as addressed below, include numerous genetic, developmental, and acquired conditions.

Ureteral flow can be shunted rather than bypassed about a diseased or otherwise obstructive segment. Shunting using conventional surgery may be to the other ureter by transureteroureterostomy, to the renal pelvis by ureteropyelostomy, to a renal calyx by ureterocalicostomy to the bladder by ureteroneocystostomy (neoureterocystostomy) with or without a vesicopsoas hitch or pull-up and psoas ureteropexy or a Boari bladder flap (http://emedicine.medscape.com/article/1893904-overview), or reconnection to the bladder through ureteral reimplantation, all long since optimized, and with the exception of the Boari flap, which as addressed below, harvests a portion of the bladder dome, to be preferred, certainly in a non-immunocompromised patient, as avoiding the need for an external collection bag (for illustrations, see, Santucci, R. A. 2017. “Ureteral Trauma Treatment & Management,” Online at http://emedicine.medscape.com/article/440933-treatment)(see also Madalina, A. O., Jerernie, S., Benoit, R., Canis, M., Revaz, B., and Bourdel, N. 2018. “Laparoscopic Ureteroneocystostomy with a Vesicopsoas Hitch in 10 Steps,” Journal of Minimally Invasive Gynecology 25(6): 951).

Ordinarily, where one ureter is missing a segment due to trauma or deformity as to require that a small segment be removed, a unilateral prosthesis as described herein is unnecessary, end-to-end anastomosis of the cut ends by ureteroureterostomy practiced universally. Defects that extend over too great a length of the ureter for end-to-end anastomosis require anastomosis of the damaged or abnormal ureter at the most proximal part of the functional urinary tract. While diversion jackets allow a ureteral obstruction to be bypassed analogously to a coarctation of the aorta, and the accessory channels can be used to release an antimicrobial, for example, most causes should be treated by accepted surgical methods, openings.

A Boari flap can be supplanted by attaching a side-entry jacket toward the end of the ureteral stump and using the accessory channel to pass urine to the bladder through a nonjacketing side-entry connector fastened at the bladder dome. The use of basic side-entry jackets, nonjacketing side-entry connectors, and diversion valves and servovalves in lieu of conventional surgical repair to bypass a diseased segment or shunt an obstruction might be appropriate where the advantage of direct drug targeting through the accessory channels is desired. Such an approach should not necessitate reentry to recover the side-entry devices.

A jacket used thus would normally have a second accessory channel to allow the direct delivery of drugs into the catheter and bladder. Ideally therefore, barring complications, even though the means described in U.S. Pat. Nos. 11,013,858 and 11,759,186 as well as herein might be used to supplant the accepted procedures altogether, the conventional repair most familiar to the operator should be done with the addition of means described in the foregoing applications and herein to target drugs or a crystal solvent, for example, directly to the wounds, anastomoses, and susceptible segment of the tract.

For this purpose, a conventional portacath, or mediport (Medi-Port®), positioned in the pectoral region is used to infuse the drug, drugs, or other agents into the accessory channel leading directly to the nonjacketing side-entry connector, side-entry jacket, or diversion valve. Where ductus side-entry jackets are for partial shunting or bypassing as pertains to channeling blood to ischemic tissue on a permanent basis, for example, diversion jackets shunt or bypass all flow through the ductus.

For dialysis or apheresis, such shunting is intermittent. The means described herein are intended for long-term use, which may involve short or long interval intermittent or multiple daily use. The synthetic to native long-term direct contact sutured interfaces of conventional polyethylene terephthalate (Dacron®, Terylene®, woven Dacron® or Terylene®), and polytetrafluoroethylene shunts and bypasses—of which the failure is a medical emergency, are susceptible to complications which the protected interfaces of ductus side-entry jackets, diversion jackets, and connectors are not.

Automatic sensor monitoring of urinary constituents, rate of output, and volume may signal the need for examination or if programed to automatically respond to such deviations, register the need for and initiate the system response. Especially when collateral functions are involved, there exists the need for a subdermally placed injection and infusion port with multiple openings to allow the direct piping of different drugs into different blood vessels or levels along the digestive tract. Ambulatory urinalysis and hemoanalysis can be used to signal er, here, as deviations in renal as well as other organ system functions as reflected in the composition of urine and/or blood, and the need to initiate delivery of remedial medication.

For example, ketoacidosis and hyperglycemia signal the need for insulin, which provided promptly, averts the need for sodium bicarbonate and the risk of hyperatremia. Significantly, the ability to directly target the delivery of drugs to specific levels of arteries and veins allows control over whether processing of drugs will precede or follow first pass processing by the liver and kidneys. As the present content pertains more narrowly to repairs of the urinary tract, the application of side-entry diversion jackets is here illustrated in terms of that organ system. Also detectable with prompt response are nitrituria, acute renal failure, hypokalemia, hyperkalemia, adrenal insufficiency as hypoaldosteronism, hypercalciuria, hyporcalciuria, hypophosphaturia, hyperparathyroidism; hypertension, diabetes mellitus, jaundice, or hyperthyroidism seen as proteinuria or albuminuria. Many other tests might be cited.

In certain circumstances, certainly where tissue is harvested creating a separate preliminary site for the risk of complications, the means described in U.S. Pat. Nos. 11,013,858 and 11,759,186, and herein can be used to avoid these. Attachment to a calyx, the pelvis, or the bladder is with a nonjacketing side-entry connector, and attachment to a ureter is with a ureteral takeoff jacket. Where the condition of the tissue for anastomosis prevents such, these means not only avert the need therefor but afford a path to the affected tissue for the direct delivery of drugs. A native or allograft ureteropyelostomy performed with a nonjacketing connector at the pelvis, or a ureterocalicostomy calyx with the connector at the calyx and side-entry jacket on the ureter eliminates the need for a partial nephrectomy at the inferior pole of the kidney.

Using the means described and illustrated in U.S. Pat. No. 11,013,858, entitled Nonjacketing Side-entry Connectors and Prosthetic Disorder Response Systems, a nephrostomy is accomplished with a nonjacketing side-entry connector with nephrostomy tube as shown in FIG. 11 run through to the pelvis with the aid of a guidewire, the nephrostomy tube connected through the most proximal intact segment along the tract. If this is to the ipsilateral ureter, a ductus side-entry jacket as described and illustrated in U.S. Pat. No. 11,759,186 is used to connect to the ureter, with the accessory channel or channels of the ureteral jacket used to deliver any needed medication. At this level, the surface port is connector with connection the same as for a nephrostomy, positioned in the pectoral region.

Takeoff from a ureter is by means of a side-entry diversion jacket as described herein, with connection either to an abdominal surface port, or if a distal portion of the tract remains intact and can be bypassed to allow normal voiding, then to that portion. If to the contralateral ureter or to a portion of the same ureter beyond the diseased or traumatized segment, then connection is by means of a ductus side-entry jacket. If to the bladder, then connection is by means of a nonjacketing side-entry connector, with placement of the surface port dependent upon the level of the diversion jacket along the ureter. Side-entry connectors, ductus side-entry jackets, and side-entry diversion jackets can all be provided with one or more accessory channels to allow the direct delivery of medication from the surface port to the site of injury or disease or the site to which the diseased or injured segment is bypassed.

Depending upon the circumstances, takeoff from a dysfunctional bladder can be by means of a synthetics-based suprapubic cystostomy connected to an abdominal surface port. Because the surface ports described herein are much thinner and less noticeable than either a stoma or a conventional portacath, the positioning of these along the body surface is much more flexible. If diversion is to a bladder that is functional or can be made to contract with the aid of electrical stimulation, then as shown in U.S. Pat. No. 11,013,858, FIG. 12C, connection is to the urethra, the need for a collection bag eliminated. Thus, while diversion as illustrated herein is to a collection bag usually cinched about a thigh, depending upon the detailed condition of the urinary tract, the need for a bag can often be eliminated.

Whether a conventional ureteropyelostomy or ureteroneocystostomy is more susceptible to complications in kidney transplantation is a matter of dispute with most references antiquated and those recent opting for ureteroneocystostomy (see, for example, Norris, J. M., Ravi-Shankar, S., and Klimach, S. G. 2015. “Urinary Reconstruction after Kidney Transplantation: Pyeloureterostomy versus Ureteroneocystostomy,” International Journal of Surgery [London] 19:83-86; Saidi. R. F., Elias, N., Hertl. M., Kawai, T., Cosimi, A., and Ko, D. S. 2013. “Urinary Reconstruction after Kidney Transplantation: Pyeloureterostomy or Ureteroneocystostomy,” Journal of Surgical Research 181(1): 156-159; Timsit, M. O., Lalloue, F., Bayramov, A., Taylor, M., Billaut, C., and 4 others 2010. “Should Routine Pyeloureterostomy be Advocated in Adult Kidney Transplantation? A Prospective Study of 283 Recipients,” Journal of Urology (Philadelphia, Pennsylvania) 184(5): 2043-2048; Donohue, J. P., Hostetter, M., Glover, J., and Madura, J. 1975. “Ureteroneocystostomy versus Ureteropyelostomy: A Comparison in the Same Renal Allograft Series,” Journal of Urology (Philadelphia, Pennsylvania) 114(2): 202-203), where the means described in the foregoing applications and herein are believed superior to both.

Much the same applies to comparisons of ureteroureterostomy versus conventional ureteroneocystostomy (see, for example Nie, Z., Zhang, K., Huo, W., Li, Q., Zhu, F., and Jin, F. 2010. “Comparison of Urological Complications with Primary Ureteroureterostomy versus Conventional Ureteroneocystostomy,” Clinical Transplantation 24(5): 615-619; Faenza, A., Nardo, B., Fuga, G., Liviano-D' Arcangelo, G., Grammatico, F., and 4 others 2005. “Urological Complications in Kidney Transplantation: Ureterocystostomy versus Uretero-ureterostomy,” Transplantation Proceedings 37(6): 2518-2520; Tan, E. C., Lim, S. M., and Rauff. A. 1991. “Techniques of Ureteric Reimplantation in Kidney Transplantation and Its Related Urological Complications,” Annals of the Academy of Medicine, Singapore 20(4): 524-528).

Considered sound by conventional standards (Jin, P., Xie, J., Dun, J., Ding, X., Zhou, C., and 4 others 2017. “Treatment of Ureteral Fistula and Stricture following Renal Transplantation with Boari Flap,” (in Chinese with English abstract at Pubmed), Zhong Nan Da Xue Xue Bao Yi Xue Ban (Journal of Central South University. Medical Sciences) 42(1): 78-82; Andrade, C., Narducci, F., Bresson, L., and Leblanc, E. 2016. “Boari Flap Ureteroneocystostomy in an Oncological Patient,” Gynecologie Oncology 143(1): 193; Ghosh, B., Biswal, D. K., Bera, M. K., and Pal. D. K. 2016. “Laparoscopic Extravesical Lich-Gregoir Ureteroneocystostomy with Psoas Hitch for the Management of Ureterovaginal Fistula in Post-Hysterectomy Patients,” Urologia Internationalis 96(2): 171-176; Wenske, S., Olsson, C. A., and Benson, M. C. 2013. “Outcomes of Distal Ureteral Reconstruction through Reimplantation with Psoas Hitch, Boari Flap, or Ureteroneocystostomy for Benign or Malignant Ureteral Obstruction or Injury,” Urology 82(1): 231-236), the Boari flap technique tubularizes a portion of the bladder dome or roof, injuring and risking complications that a ureteral diversion jacket and drainage catheter emptying into the otherwise intact bladder through a nonjacketing side-entry connector would avoid, as well as allow the direct targeting of drugs to the site from a portacath.

Reports of successful results initially may not specify long-term outcomes beyond three months (see, for example, Leitão, T. P., E Silva, R. P., Barata, S., Guerra, A., and Osorio, F. 2016. “Laparoscopic Psoas Hitch Double Ureteral Re-implantation in the Duplex Urinary System for Treatment of Ureterovaginal Fistula,” Surgical Technology International 28: 170-176; Yang. C., Jones, L., Rivera, M. E., Verlee, G. T., and Deane, L. A. 2011. “Robotic-assisted Ureteral Reimplantation with Boari flap and Psoas Hitch: A Single-institution Experience,” Journal of Laparoendoscopic Advanced Surgical Techniques A 21(9): 829-833).

Diversion with a nonjacketing side-entry connector or ductus side-entry jacket and catheteric drugline described in U.S. Pat. No. 11,013,858, entitled Nonjacketing Side-entry Connectors and Prosthetic Disorder Response Systems, and 11,759,186, entitled Ductus Side-entry Jackets and Prosthetic Disorder Response Systems, or a simple side-entry diversion jacket as described herein still have a distinct advantage in allowing the direct targeting to the treatment site of drugs. Certain complications also recommend the use of synthetics over reconstructions.

These include a transureteroureterostomy when the risk of injury to the recipient ureter is high (Sandoz, I. L., Paull. D. P., and MacFarlane, C. A. 1977. “Complications with Transureteroureterostomy,” Journal of Urology (Philadelphia, Pennsylvania) 117(1): 39-42). While more successful than most other reconstructive procedures, distal obstruction, recurrent stricture, and leakage may necessitate more than one follow-up procedure (Iwaszko, M. R., Krambec, A. E., Chow, G. K., and Gettman, M. T. 2010. “Transureteroureterostomy Revisited: Long-term Surgical Outcomes,” Journal of Urology (Philadelphia, Pennsylvania) 183(3): 1055-1059; Strup, S. E., Sindelar, W. F., and Walther, M. M. 1996. “The Use of Transureteroureterostomy in the Management of Complex Ureteral Problems,” Journal of Urology (Philadelphia, Pennsylvania) 155(5): 1572-1574).

For refractory urinary incontinence, either the nonadjustable (prosthetic) embodiment when incurable, and the adjustable embodiment when correctable can be used in lieu of an indwelling Foley catheter, eliminating restriction on the duration of continuous use of a single device, as well as numerous complications, primarily of infection, but also discomfort, genitourinary trauma, clogging, leaking, hematuria, retention and fragmentation, cystolithiasis, pyelonephritis, urosepsis, bacterial endocarditis, and problems due to breakage during insertion and removal (see, for example, Gray, M., Skinner, C., and Kaler, W. 2016. “External Collection Devices as an Alternative to the Indwelling Urinary Catheter: Evidence-Based Review and Expert Clinical Panel Deliberations,” Journal of Wound, Ostomy, and Continence Nursing 43(3): 301-307; Feneley, R. C., Hopley, I. B., and Wells, P. N. 2015, Op cit.; Aziz, e1 M., Amrani, M., Abdelhak, K., and Hassan F. M. 2015. “A Fragment of Foley Catheter Balloon as a Cause of Bladder Stone in Woman,” Online, Pan African Medical Journal (Kampala, Uganda) 21:284; Leuck. A/M., Wright. D., Ellingson, L., Kraemer, L., Kuskowski, M. A., and Johnson, J. R. 2012. “Complications of Foley Catheters—Is Infection the Greatest Risk?,” Journal of Urology (Philadelphia, Pennsylvania) 2012 187(5): 1662-1666; Ho. C. C., Khandasamy, Y., Singam, P., Hong Goh, E., and Zainuddin, Z. M. 2010. “Encrusted and Incarcerated Urinary Bladder Catheter: What are the Options?,” Libyan Journal of Medicine 5; Hamilton, R. J., Jewett, M. A., and Finelli, A. 2006. “An Efficient Solution to the Retained Foley Catheter,” Urology 68(5): 1109-1111; Patterson, R., Little, B., Tolan, J., and Sweeney, C. 2006. “How to Manage a Urinary Catheter Balloon that Will Not Deflate,” International Urology and Nephrology 38(1): 57-61; Hollingsworth, M., Quiroz, F., and Guralnick, M. L. 2004. “The Management of Retained Foley Catheters,” Canadian Journal of Urology 11(1): 2163-2166; Chrisp, J. M. and Nacey, J. N. 1990. “Foley Catheter Balloon Puncture and the Risk of Free Fragment Formation,” British Journal of Urology 66(5): 500-502). Where incontinence affects an otherwise intact lower urinary tract, the addition of an intracystic urethra-noncompressive continence device with directly targeted therapeutic support, application to be filed shortly, allows the wearer to use an adjustable diversion device in the open position during the day without a collection bag.

External urinary drainage devices (single use or Texas, urisheath/uriliner, “condom catheter”, diver's drysuit p-valve) are less problematic than urethral catheters, especially used over a longer period, but still pose risks in addition to infection. Long-term use, especially when not properly fitted and maintained, can lead to ulceration, necrosis, urethrocutaneous fistulae, urethral diverticulum, and fibroepithelial polyp of the prepuce (see, for example, Bird. V. Y., Chastain-Gross, R., R., Bird. V. G., Vyas. P., and Joseph, R. 2017. “Pseudomonas aeruginosa as an Etiologic Agent of Nephrolithiasis in Deep Water Divers,” Journal of Endourology Case Reports 3(1): 4-6; Özkan, H. S., Irkoren, S., and Sivrioğlu, N. 2015. “Penile Strangulation and Necrosis Due to Condom Catheter,” International Wound Journal 12(3): 248-249; Mason, S. E., De Villiers, P., and Andea, A. A. 2009. “Lymphedematous Fibroepithelial Polyps of the Penis Associated with Long-term Condom Catheter Use: Case Report and Rreview of the Literature,” Journal of Cutaneous Pathology 36(8): 906-909; Harris, R. 2009. “Genitourinary Infection and Barotrauma as Complications of ‘P-valve’ Use in Drysuit Divers,” Diving and Hyperbaric Medicine 39(4): 210-212; Banerji, J. S., Shah, S., and Kekre, N. S. 2008. “Fibroepithelial Polyp of the Prepuce: A Rare Complication of Long-term Condom Catheter Usage,” Indian Journal of Urology 24(2): 263-264; Jayachandran, S., Mooppan, U. M., and Kim, H. 1985. “Complications from External (Condom) Urinary Drainage Devices,” Urology 25(1): 31-34). Improvised such devices can result in servere complications (see, for example, Paul, S., Dalela, D., Prakash, J., and Sankhwar, S. 2013. “Penile Elephantiasis: A Rare Consequence of Inappropriate Use of Condom as External Urinary Collection Receptacle,” Online, BMJ [British Medical Journal] Case Reports 2013).

Implantation of the devices to be described for permanent use supplants the use of conventional urinary catheters of all types for long-term use, which overcome the significant problems associated with noncompliant use, even by patients and healthcare personnel, who may be competent but neglectful (see, for example, Wilde, M. H., Crean, H. F., McMahon, J. M., McDonald, M. V., Tang, W., and 4 others 2016. “Testing a Model of Self-Management of Fluid Intake in Community-Residing Long-term Indwelling Urinary Catheter Users,” Nursing Research 65(2): 97-106; Feneley, R. C., Hopley, I. B., and Wells. P. N. 2015, Op cit.; Taleschian-Tabrizi, N., Farhadi, F., Madani, N., Mokhtarkhani, M., Kolahdouzan, K., and Hajebrahimi, S. 2015. “Compliance with Guideline Statements for Urethral Catheterization in an Iranian Teaching Hospital,” International Journal of Health Policy and Management 4(12): 805-811; Wilde, M. H., McMahon, J. M., McDonald, M. V., Tang. W., Wang. W., and 5 others 2015. “Self-management Intervention for Long-term Indwelling Urinary Catheter Users: Randomized Clinical Trial,” Nursing Research 64(1): 24-34; Nicolle, L. E. 2014. “Catheter Associated Urinary Tract Infections,” Antimicrobial Resistance and Infection Control 3:23; Seth, J. H., Haslam, C., and Panicker, J. N. 2014. “Ensuring patient Adherence to Clean Intermittent Self-catheterization,” Patient Preference and Adherence 8:191-198; Codd, J. 2014. “Implementation of a Patient-held Urinary Catheter Passport to Improve Catheter Management, by Prompting for Early Removal and Enhancing Patient Compliance,” Journal of Infection Prevention 15(3): 88-92; Wilde, M. H., McDonald, M. V., Brasch, J., McMahon, J. M., and 4 others 2013. “Long-term Urinary Catheter Users Self-care Practices and Problems,” Journal of Clinical Nursing 22(3-4): 356-367; Royal College of Physicians 2012. “Infection: Prevention and Control of Healthcare-Associated Infections in Primary and Community Care,” National Clinical Guideline Centre Guidelines, Number 139; Hola, V., Peroutkova, T., and Ruzicka, F. 2012. “Virulence Factors in Proteus Bacteria from Biofilm Communities of Catheter-associated Urinary Tract Infections,” FEMS [Federation of European Microbiological Societies] Immunology and Medical Microbiology 65(2): 343-349).

Combining a lower abdominal or pelvic, that is, subcutaneously implanted body surface port to a side of the mons pubis or mons veneris, which excurrent to emit urine, must provide an opening to the exterior, with a subcutaneously placed, totally implanted port implanted in the pectoral region for targeting drugs through its accessory channel to the ureterally positioned diversion valve can be argued for as reducing the risk of infection as can the design of the port which allows the direct application of antimicrobials or antiseptic (see, for example Gray, M., Skinner, C., and Kaler, W. 2016. “External Collection Devices as an Alternative to the Indwelling Urinary Catheter: Evidence-Based Review and Expert Clinical Panel Deliberations,” Journal of Wound, Ostomy, and Continence Nursing 43(3): 301-307; Kulkarni, S., Wu, O., Kasthuri, R., and Moss, J. G. 2014. “Centrally Inserted External Catheters and Totally Implantable Ports for the Delivery of Chemotherapy: A Systematic Review and Meta-analysis of Device-related Complications,” Cardiovascular and Interventional Radiology 37(4): 990-1008). Cutaneous, that is, above-skin injection incurrent openings just beneath the port outer dome cap, are covered over by a self-sealing silicone membrane, or septum, (Yoo, J. J., Magliochetti, M., and Atala, A. 1997. “Detachable Self-sealing Membrane System for the Endoscopic Treatment of Incontinence,” Journal of Urology (Philadelphia, Pennsylvania) 158(3 Part 2): 1045-1048) during use, then covered over by closing the hinged port dome cap. Open and closed, the port is wetted with an antimicrobial or antiseptic, and subdermal ports are closed off from the environment, so that pathogens can enter only on the needles or cabled devices that are passed through the skin.

Since as with any injection, the skin is swabbed with an antiseptic before insertion, the likelihood of microbial invasion is slight. However, combining these in a single port with excurrent urine outlet and subdermally positioned injection openings for drugs eliminates the need for a second port. The surface port not only replaces a stoma, itself the site of numerous complications demanding constant maintenance, but serves as an entry portal for the direct targeting of medication, maintenance agents, and the passage of cabled devices such as scopes and lasers through the apparatus. Where no excurrent flow is required as it is with urinary diversion, the port is completely subdermal. Any difficulty in distinguishing different entry holes in the port by palpating the slight bump the port makes at the surface is clarified by tatooing tiny dot indicators.

While seldom exposed, hence, unlikely to pose a cosmetic problem, if the urinary tract no longer requires a prosthetic outflow path, or exogenous or therapeutic support, the dots are readily removed by laser or mini-punch skin excision (see, for example, Baumler, W. and WeiB, K. T. 2019. “Laser Assisted Tattoo Removal—State of the Art and New Developments,” Photochemical and Photobiological Sciences 18(2): 349-358; Gupta, J., Chouhan, K., Kumar, A., and Garg. K. 2019. “Mini-punch Skin Excision: A Newer Novel Approach for Tattoo Removal,” Journal of Cutaneous and Aesthetic Surgery 12(1): 49-51; Henley, J. K and Ramsey, M. L. 2019. “Laser Tattoo Removal,” Treasure Island, Florida StatPearls Publishing, Online at https://www.ncbi.nlm.nih.gov/books/NBK442007/; Sardana, K., Ranjan, R., and Ghunawat, S. 2015. “Optimising Laser Tattoo Removal,” Journal of Cutaneous and Aesthetic Surgery 8(1): 16-24; Ho. S. G. and Goh, C. L. 2015. “Laser Tattoo Removal: A Clinical Update,” Journal of Cutaneous and Aesthetic Surgery 8(1): 9-15).

General information pertaining to ductus side-entry jackets and diversion valves is provided in section 2a. The urine diversion chute situated behind the trepan shown in the drawing figures is retracted during, and little if at all interferes with plug extraction. In prosthetic, that is, nonadjustable, or fixed embodiments, once the tissue plug is removed, the diversion chute is advanced into luminal position by pushing on the accessory channel until male and female detents on the trepan floor and beneath the diversion chute engage with an audible click.

With an intermittent embodiment, placement is likewise with the diversion chute in the retracted position, except that either push-pull solenoids or a Bowden or push/pull control cable adjusted at the surface port is used. The adjustable embodiment can be used to target a drug or drugs to the lower tract at the same time that urine is diverted away from the lower tract, which bypassed, is kept free of contract with urine, which is caustic, and thus afforded an improved opportunity to heal following the initiation of treatment for a bladder dysfunction or surgical procedure.

Exposure of urothelium to urine, which contains “ . . . harmful and noxious substances that must be stored for hours . . . ” can leave it defective for having undergone “ . . . urothelial barrier breakdown and developed” . . . inappropriate signaling from urothelial cell to underlying sensory afferents and potentially interstitial cells . . . ,” resulting in lower urinary tract dysfunction, to include frequency, urgency, incontinence, overactivity, and pain (Hill. W. G. 2015. “Control of Urinary Drainage and Voiding,” Clinical Journal of the American Society of Nephrology 10(3): 480-492)(see also, for example. Chakrabarty. B., Bijos, D. A., Vahabi, B., Clavica, F., Kanai, A. J., and 3 others 2019. “Modulation of Bladder Wall Micromotions Alters Intravesical Pressure Activity in the Isolated Bladder,” Online, Frontiers in Physiology 9:1937; Fry, C. H. and McCloskey, K. D. 2019. “Spontaneous Activity and the Urinary Bladder,” Advances in Experimental Medicine and Biology 1124:121-147; Iguchi, N., Donmez, M. I., Carrasco, A. Jr., Wilcox, D. T., Pineda, R. H., Malykhina, A. P., and Cost. N. G. 2019. “Doxorubicin Induces Detrusor Smooth Muscle Impairments through Myosin Dysregulation, Leading to a Risk of Lower Urinary Tract Dysfunction,” American Journal of Physiology. Renal Physiology 317(1): F197-F206; Lee, S. M. and Hashim, H. 2018. “Recent Advances in the Understanding and Management of Underactive Bladder,” Faculty of 1000 Research 7:437; Drake, M. J., Kanai. A., Bijos, D. A., Ikeda, Y., Zabbarova, I., Vahabi, B., and Fry, C. H. 2017. “The Potential Role of Unregulated Autonomous Bladder Micromotions in Urinary Storage and Voiding Dysfunction; Overactive Bladder and Detrusor Underactivity,” British Journal of Urology International 119(1): 22-29; Vahabi. B. and Drake, M. J. 2015. “Physiological and Pathophysiological Implications of Micromotion Activity in Urinary Bladder Function,” Acta Physiologica (Oxford, England) 213(2): 360-370; Chacko, S., Cortes, E., Drake, M. J., and Fry, C. H. 2014. “Does Altered Myogenic Activity Contribute to OAB [overactive (urinary) bladder] Symptoms from Detrusor Overactivity? ICI-RS 2013 [2013 International Consultation on Incontinence-Research Society (ICI-RS) meeting in Bristol, UK],” Neurourology and Urodynamics 33(5): 577-580; Kanai, A., Fry. C., Hanna-Mitchell, A., Birder, L., Zabbarova, I., Bijos, D., and Ikeda, Y. 2014. “Do We Understand any More about Bladder Interstitial Cells?-ICI-RS 2013 [2013 International Consultation on Incontinence-Research Society (ICI-RS) meeting in Bristol, UK],” Neurourology and Urodynamics 33(5): 573-576; Rasouly, H. M. and Lu, W. yr765 2013. “Lower Urinary Tract Development and Disease,” Wiley Interdisciplinary Reviews. Systems Biology and Medicine 5(3): 307-342).

During placement of the diversion valve, the opposing wall of the ureter is kept from being drawn against the trepan by insufflation at the least pressure needed to keep the opposite urothelial surface at a safe distance. That with the system described, doxorubicin would be directly pipeline targeted to the original or root-site of disease, avoiding its life-threatening side effects, to include dilated cardiomyopathy tending toward congestive heart failure, neutropenic enterocolitis, or typhlitis, and less severe palmar plantar erythrodysesthesia, or chemotherapy-induced acral erythema even at elevated dosages, should be clear.

Insertion of a ureteral stent or Foley catheter to cover over the urothelial lining protects it from urine but also makes it inaccessible to the direct targeting of medication through a side-entry diversion valve. In this application, urine is diverted from the ureter or ureters and is passed directly into a paracorporeal collection bag having been detoured about the affected lower tract. At the same time, medication flows in through an accessory (service channel, sideline, drugline) to wet the ureteral urothelium to expedite recovery while spared exposure to urine.

The foam lining of the diversion valve takeoff jacket allows the diversion valve to be positioned at any level along the ureter. Moreover, where the ductus deferens or broad ligament of the uterus cross the ureter, the dimensions of the trepan and diversion chute allow avoiding any constriction; instead, the jacket is placed slightly above (cephalad, craniad, superior) or below (caudad, inferior) to this small segment. Other points of constriction along the ureter (see, for example, Jendeberg, J., Geijer, H., Alshamari, M., and Liden, M. 2018. “Prediction of Spontaneous Ureteral Stone Passage: Automated 3D-measurements Perform Equal to Radiologists, and Linear Measurements Equal to Volumetric,” European Radiology 28(6): 2474-2483; Eisner, B. H., Reese, A., Sheth, S., and Stoller, M. L. 2009. “Ureteral Stone Location at Emergency Room Presentation with Colic,” Journal of Urology (Philadelphia, Pennsylvania) 182(1): 165-168) can if difficult for placement of a diversion jacket can be connected through a miniature nonjacketing side-entry connector and catheter, as delineated in U.S. Pat. No. 11,013,858.

At 2.0 millimeters in diameter, the trepan, hence, the hole it cuts, is substantially the same in diameter as the mean internal diameter of the adult ureteral (ureteric) lumen when passing a bolus. The same jacket is used for the average adult human blood vessel. In distinguishing fixed prosthetic from intermittent use embodiments, it warrants emphasis that a spectrum of conditions lies between these extremes. For this reason, either type embodiment incorporates some features that would support the purpose of the other. For example, the fixed embodiment incorporates an accessory channel which is connected to a groove running along the center underside of the diversion chute and end at the distal tip thereof inside the native lumen, and a set-once manually operated flow, or prosthetic diversion, valve can actually be retracted, but requires more force.

Accessory channels afford paths for the direct targeting of drugs to any portion of the ureter or other part of the lower urinary tract that may require medication. For example, the tissue of the dysfunctional lower tract might become infected and heal more quickly with the direct (surface-wetting, topical) application of an antimicrobial, anti-inflammatory, and/or anesthetic where appropriate, allowing a background systemic dose much reduced if still needed at all.

For use along the urinary tract, where flow through the ureter is less forceful than that pulsatile under pressure encountered with an artery (see, for example, Osman, F., Romics, I., Nyirady, P., Monos, E., and Nadasy, G. L. 2009. “Ureteral Motility,” Acta Physiologica Hungarica 96(4): 407-426; Tillig, B., Mutschke, O., Rolle, U., Gaunitz, U., Asmussen, G., and Constantinou, C. E. 2004. “Effects of Artificial Obstruction on the Function of the Upper Urinary Tract of Guinea Pigs, Rats and Pigs,” European Journal of Pediatric Surgery 14(5): 303-315; Ohlson, L. 1989. “Morphological Dynamics of Ureteral Transport. I. Shape and Volume of Constituent Urine Fractions,” American Journal of Physiology 256(1 Part 2): R19-R28; Griffiths, D. J. 1989. “Flow of Urine through The Ureter: A Collapsible, Muscular Tube Undergoing Peristalsis,” Journal of Biomechanical Engineering 111(3): 206-211; Morita, T., Wada, I., Saeki, H., Tsuchida, S., and Weiss, R. M. 1987. “Ureteral Urine Transport: Changes in Bolus Volume, Peristaltic Frequency, Intraluminal Pressure and Volume of Flow Resulting from Autonomic Drugs,” Journal of Urology (Philadelphia, Pennsylvania) 1987 137(1): 132-135; Saeki, H., Morita, T., Weiss, R. M., and Miyagawa, I. 1986. “The Role of Ureteral Peristaltic Rate and Bolus Volume on Increasing Urine Flow,” Urologia Internationalis 41(3): 174-179), the problems of apparatus leakage or inflow can be averted.

This is by administering an antidiuretic, or oliguric, drug such as a shorter acting antidiuretic hormone and octapeptide vasopressins (see, for example, Ritter, J., Flower, R., Henderson, G., and Rang, H. 2016. “The Kidney and Urinary System,” Chapter 29 in Rang & Dale's Pharmacology London, England: Elsevier/Churchill Livingstone; Yadav, R. K., Nandy, B. C., Maity, S., Sarkar, S., and Saha, S. 2015. “Phytochemistry, Pharmacology, Toxicology, and Clinical Trial of Ficus Racemosa,” Pharmacognosy Reviews (Mumbai [Bombay], India) 9(17): 73-80; El-Farhan, N., Hampton, D., and Penney, M. 2013. “Measurement of Arginine Vasopressin,” Methods in Molecular Biology 1065:129-139; Ratnasooriya, W D., Jayakody, J. R., and Nadarajah, T. 2003. “Antidiuretic Activity of Aqueous Bark Extract of Sri Lankan Ficus Racemosa in Rats,” Acta Biologica Hungarica 54(3-4): 357-363; Abrahams, V. C. and Pickford, M. 1956. “The Effect of 5-hydroxytryptamine on the Ureter and on the Blood Pressure of Dogs; and of Adrenaline, Noradrenaline, and Posterior Pituitary Extracts on the Ureter,” British Journal of Pharmacology and Chemotherapy 11(1): 44-49) and placement of a cross-clamp over the brief interval it takes to place the jacket.

As antidiuretics, carbamazepine and chlorpropamide must be systemically circulated to reach the neurohypophysis. Exposing the brain as well, these drugs are unsuitable for direct targeting of the urinary tract, of which the object would be precisely to bypass the central nervous system. Furthermore, systemically administered, chlorpropamide, now seldom prescribed to treat diabetes, is contraindicated for use in the obese and elderly, and carbamazepine, used primarily for neuropsychiatric disorders, is no less likely to cause more harm than good (see, for example, Sola, D., Rossi, L., Schianca, G. P., Maffioli, P., Bigliocca, M., and 5 others 2015. “Sulfonytureas and Their Use in Clinical Practice,” Archives of Medical Science 11(4): 840-848; Kang, D. I., Kim, H. M., Oh, S. Y., Yoon, J. H., Kim, H. M., and Min, K. S. 2010. “Short Term Effect and Safety of Antidiuretic Hormone in the Patients with Nocturia,” International Neurourology Journal 14(4): 227-231; Tormey, W. P. 1993. “Mechanisms of Carbamazepine-induced Antidiuresis,” Journal of Neurology. Neurosurgery, and Psychiatry 56(5): 567).

If necessary, once the jacket has been placed, an antidiuretic drug reversal agent is administered. The administration or withholding of an antidiuretic is a clinical judgement based upon the general health and drug regimen of the patient, especially with respect to diabetes. Blood is prevented from extravasation only while the jacket is being placed; once placed, the shunt or bypass created allows the free outflow of blood to the target tissue. Accordingly, a diversion type side-entry jacket for placement along a blood vessel but not one for placement along a ureter must incorporate a water jacket as shown in U.S. Pat. No. 11,759,186, entitled Ductus Side-entry Jackets and Prosthetic Disorder Response Systems.

Conventionally, a nephrostomy is used as a temporary bridging or holdover measure pending surgical correction where trauma or deformity of the distal urinary tract prevents urinary diversion at or to a lower and considerably less physiologically complex and heavily vascularized, hence, risk-prone level. U.S. Pat. No. 11,013,858, entitled Nonjacketing Side-entry Connectors and Prosthetic Disorder Response Systems, describes a method and apparatus for creating a permanent nephrostomy using synthetic materials. Unless an intact bladder is present or can be created, diversion is to a conventional paracorporeal, or externally worn collection bag. However, urinary diversion, or takeoff, at the kidney or kidneys assumes that no less desperate alternative is offered at a lower level.

Whenever possible, takeoff at the ureter avoids the greater risk incurred with interception at the kidney, which includes hemorrhage, infection, septic shock, iatrogenic injury to include renal puncture damage, increased time to establish renal drainage, coagulopathy, and “ . . . hematoma, pseudoaneurysm, arteriovenous fistula, injury and disruption of the renal pelvicalyceal (pelvicaliceal) system, arteriocalyceal (arteriocaliceal) fistula, and renal foreign bodies such as retained wires, sponges, or other surgical or endourological instruments” (Esparaz, A. M., Pearl, J. A., Herts, B. R., LeBlanc, J., and Kapoor, B. 2015. “Iatrogenic Urinary Tract Injuries: Etiology, Diagnosis, and Management,” Seminars in Interventional Radiology 32(2): 195-208); (also see, for example, Gross, J. A., Lehnert, B. E., Linnau, K. F., Voelzke, B. B., and Sandstrom, C. K. 2015. “Imaging of Urinary System Trauma,” Radiologic Clinics of North America 53(4): 773-788; Patel, B. N. and Gayer, G. 2014. “Imaging of Iatrogenic Complications of the Urinary Tract: Kidneys, Ureters, and Bladder,” Radiologic Clinics of North America 52(5): 1101-1116; Lewis, S. and Patel, U. 2004. “Major Complications after Percutaneous Nephrostomy-Lessons from a Department Audit” Clinical Radiology 59(2): 171-179; Kaskarelis, I. S., Papadaki, M. G., Malliaraki, N. E., Robotis, E. D., Malagari, K. S., and Piperopoulos, P. N. 2001. “Complications of Percutaneous Nephrostomy, Percutaneous Insertion of Ureteral Endoprosthesis, and Replacement Procedures,” Cardiovascular and Interventional Radiology 24(4): 224-228).

The risks of percutaneous nephrostomy are greater when the kidney with ureter is a transplant and a functional bladder missing, for example, the diversion jacket is positioned above would otherwise be an end-to-end ureterocystic anastomosis (see, for example, Sabnis, R. B., Singh, A. G., Ganpule, A. P., Chhabra, J. S., Tak, G. R., and Shah, J. H. 2016. “The Development and Current Status of Minimally Invasive Surgery to Manage Urological Complications after Renal Transplantation,” Indian Journal of Urology 32(3): 186-191; Duty, B. D., Conlin, M. J., Fuchs, E. F., and Barry, J. M. 2013. “The Current Role of Endourologic Management of Renal Transplantation Complications,” Advances in Urology 2013:246520; Mahdavi Zafarghandi, R. and Sheikhi, Z. 2013. “Extensive Ureteral Stricture in Renal Transplant Recipients: Prevalence and Impact on Graft and Patient Survival,” International Journal of Organ Transplantation Medicine 4(4): 165-171; Akoh, J. A., Opaluwa, A. S., and Weller, D. 2009. “Urological Complications of Renal Transplantation: Reducing the Risk,” Saudi Journal of Kidney Diseases and Transplantation (Mumbai [Bombay], India) 20(6): 1005-1009. Yigit. B., Tellioglu, G., Berber, I., Aydin, C., Kara, M., Yanaral. F., and Titiz, I. 2008. “Surgical Treatment of Urologic Complications after Renal Transplantation,” Transplantation Proceedings 40(1): 202-204; Dinckan, A., Tekin, A., Turkyilmaz, S., Kocak, H., Gurkan, A., Erdogan, O., Tuncer, M., and Demirbas, A. 2007. “Early and Late Urological Complications Corrected Surgically following Renal Transplantation,” Transplant International 20(8): 702-707; Aytekin, C., Boyvat, F., Harman, A., Ozyer, U., Colak, T., and Haberal, M. 2007. “Percutaneous Therapy of Ureteral Obstructions and Leak after Renal Transplantation: Long-term Results,” Cardiovascular and Interventional Radiology 30(6): 1178-1184; Burmeister, D., Noster, M., Kram, W., Kundt. G., and Seiter, H. 2006. “Urological Complications after Kidney Transplantation,” (in German with English abstract at Pubmed), Urologe Ausgabe A 45(1): 25-31; Fontaine, A. B., Nijjar, A., and Rangaraj, R. 1997. “Update on the Use of Percutaneous Nephrostomy/Balloon Dilation for the Treatment of Renal Transplant Leak/Obstruction,” Journal of Vascular and Interventional Radiology 8(4): 649-653).

Using ureteral takeoff by means of a diversion side-entry diversion jacket rather than percutaneous nephrostomy requires that the ureteral obstruction be so positioned as to afford sufficient length in the post pelvic ureter to position the jacket; however, post-transplantation stricture most often ensues at the ureterovesical anastomosis, and the jacket, millimetric in dimensions, this requirement will be satisfied. U.S. Pat. Nos. 11,013,858 and 11,759,186 address the concept of directly targeting immunosuppressant drugs to the arterial supply of the transplant organ rather than exposing the entire body to such drugs, increasing susceptibility to disease.

For temporary use, such as to allow diagnostics preparatory to prospectively curative therapy not requiring continued monitoring, the tract is emptied of urine, the ureteral diversion jacket removed, the aperture created cleared of any fibrosed tissue that may have formed about the periphery of the 2.0-millimeter aperture in an adult, and the aperture then allowed to heal, preferably by first, or if necessary, second intention. First intention is by closure with an adhesive such as surgical cyanoacrylate cement and wrapping about the wound with absorbable tape; second intention by wrapping about the ureter without first closing the wound but wrapping about the ureter with a strip of absorbable tape or artificial skin, and allowing the aperture to fill in by second intention.

Requiring entry through a ‘keyhole’ incision, takeoff at the ureter is no more, and probably less, risk prone than are the existing alternatives of a percutaneous nephrostomy, which is not long-term, much less ambulatory, or either upstream or downstream insertion of an indwelling ureteric J- or double J-stent (see, for example. Hsu, L., Li, H., Pucheril, D., Hansen, M., Littleton, R., Peabody, J., and Sammon, J. 2016. “Use of Percutaneous Nephrostomy and Ureteral Stenting in Management of Ureteral Obstruction,” World Journal of Nephrology 5(2): 172-181; ElSheemy, M. S., Shouman, A. M., Shoukry, A. I., ElShenoufy, A., Aboulela, W., and 4 others 2015. “Ureteric Stents vs Percutaneous Nephrostomy for Initial Urinary Drainage in Children with Obstructive Anuria and Acute Renal Failure Due to Ureteric Calculi: A Prospective, Randomised Study,” BJU [British Journal of Urology] International 115(3): 473-479; Ahmad, I., Saeed Pansota, M., Tariq, M., Shahzad Saleem, M., Ali Tabassum, S.5., and Hussain, A. 2013. “Comparison between Double J (DJ) Ureteral Stenting and Percutaneous Nephrostomy (PCN) in Obstructive Uropathy,” Pakistan Journal of Medical Sciences (Karachi, Pakistan) 29(3): 725-729), which rarely can result in intraoperative iatrogenic ureteric or renal parenchymal perforation on placement (Altay, B., Erkurt, B., Kiremit, M. C., and Güzelburç, V. 2015. “A Rare Complication of Ureteral Double-J Stenting after Flexible Ureteroscopy: Renal Parenchymal Perforation,” Turkish Journal of Urology (Istanbul, Turkey) 41(2): 96-98; Pradhan, A. and Rana, Y. P. 2014. “An Uncommon Situation of Kidney Perforation with a Ureteric Stent,” Central European Journal of Urology (Warsaw, Poland) 67(1): 113-114; Gönülalan, U., Akand, M., Hasιrci, E., and Koşan, M. 2014. “An Unusual Complication of a Double-J Ureteral Stent: Renal Parenchymal Perforation in a Solitary Kidney,” Turkish Journal of Urology (Istanbul, Turkey) 40(4): 245-247; Choi, S. K., Lee, S., Kim, S., Kim, T. G., Yoo. K. H., Min, G. E., and Lee, H. L. 2012. “A Rare Case of Upper Ureter Rupture: Ureteral Perforation Caused by Urinary Retention,” Korean Journal of Urology (Seoul, South Korea) 53(2): 131-133; Nomikos, M. S., Chousianitis, Z., Georgiou, C., Georgellis, C., Rikas, P., and Anagnostou, T. 2012. “Renal Parenchyma Perforation and Hematoma Formation following Double-J Stent Insertion in a Solitary Functioning Kidney: An Unusual Complication,” Case Reports in Urology 2012:301275; Carroll, D., Chandran, H., Joshi, A., Mccarthy, L. S., and Parashar, K. 2010. “Endoscopic Placement of Double-J Ureteric Stents in Children as a Treatment for Primary Obstructive Megaureter,” Urology Annals (Mumbai [Bombay], India) 2(3): 114-118; Dundar, M., Caliskan, T., and Koçak, I. 2008. “Unexpected Complication: Renal Parenchymal Perforation with Double-J Ureteral Stent,” Urological Research 36(5): 279-281) and complications once placed, to include fragmentation, “ . . . stent migration, encrustation, and fracture,” (Rao, A. R., Alleemudder, A., Mukerji, G., Mishra, V., Motiwala, H., Charig. M., and Karim, O. M. 2011. “Extra-anatomical Complications of Antegrade Double-J Insertion,” Indian Journal of Urology (Mumbai [Bombay], India) 27(1): 19-24).

Neither ureteric stents nor a percutaneous nephrostomy can remain in place for more than a short time, and cannot be exchanged interminably so as to be considered permanent treatments. Approach from outside the ureter with a jacket is less susceptible to perforation than is transluminal insertion. However, stone induced reflux resistant to lithotripsy is best served with the mechanical clearing of the lumen afforded by a stent.

Additional reports in the literature of ureteral stent-related complications are numerous (see, for example, Hikmet, T., Sercan, S., Ugur. O-H., Burhan, B., Harun, O., and 3 others 2015. “A New Method for Fragmented Ureteral Stent Extraction: Flexible Renoscopy,” Urology Case Reports 3(6): 190-192; Farshi, A. R. and Roshandel, M. R. 2015. “Intracardiac Migration of Ureteral Double-J Stent: A Case Report and Review,” Canadian Urological Association Journal 9(9-10): E661-E664; Sabnis, R. B., Ganpule, A. P., and Ganpule, S. A. 2013. “Migration of Double J Stent into the Inferior Vena Cava and the Right Atrium,” Indian Journal of Urology 29(4): 353-354; Ahallal, Y., Khallouk, A., El Fassi, M. J., and Farih, M. H. 2010. “Risk Factor Analysis and Management of Ureteral Double-j Stent Complications,” Reviews in Urology 12(2-3): e147-e151; Ivica, S. and Dragan, S. 2009. “Long-term Indwelling Double-J Stents: Bulky Kidney and Urinary Bladder Calculosis, Spontaneous Intraperitoneal Perforation of the Kidney and Peritonitis as a Result of “Forgotten” Double-J Stent,” Vojnosanitetski pregled [Military-Medical and Pharmaceutical Review] (Belgrade, Serbia) 66(3): 242-244).

In a hospital setting, percutaneous nephrostomy does, however, allow independent urinary diversion from either kidney into a separate external collection bag, and therewith, the ability to monitor the function of each kidney for renal uropathy, that is, abnormal differential function whether one kidney is infected, physiologically impaired, injured, obstructed due to a stone or structural defect, or has been operated on or transplanted (see, for example, Sharma, U., Yadav, S. S., and Tomar, V. 2015. “Factors Influencing Recoverability of Renal Function after Urinary Diversion through Percutaneous Nephrostomy,” Urology Annals 7(4): 499-503) through separate urinalysis as an adjunct to:
a. Renal biopsy (Kumar, K., Ahmad, A., Kumar, S., Choudhry, V., Tiwari, R. K., Singh, M., and Muzaffar, M. A. 2015. “Evaluation of Renal Histopathological Changes, as a Predictor of Recoverability of Renal Function following Pyeloplasty for Ureteropelvic Junction Obstruction,” Nephrourology Monthly 7(4): e28051; Bhat, G. S., Maregowda, S., Jayaram, S., and Siddappa, S. 2012. “Is Renal Biopsy a Better Predictor of the Outcome of Pyeloplasty in Adult Ureteropelvic Junction Obstruction?,” Urology 79(2): 321-325).
b. Dynamic renal scintigraphy (see, for example, Murari, S. B., Gadepalli, T., Rao, V. P., and Ram, R. 2012. “Renal Scintigraphy in Diagnosis and Management of Nephroptosis, [floating kidney, renal ptosis, dropping down of a kidney more than five centimeters upon standing]” Indian Journal of Nuclear Medicine 27(1): 52-54; Listewnik, M. H., Peregud-Pogorzelski, J., Birkenfeld, B., Matacz, M., Zorga, P., Szychot, E., Piwowarska-Bilska, H., and Brodkiewicz, A. 2012. “Dynamic Renal Scintigraphy with 99mTc-DTPA for the Assessment of Renal Function after Therapy of Acute Lymphoblastic Leukemia in Children,” Annales Academiae Medicae Stetinensis 58(1): 49-54; Caglar, M., Gedik, G. K., and Karabulut, E. 2008. “Differential Renal Function Estimation by Dynamic Renal Scintigraphy: Influence of Background Definition and Radiopharmaceutical,” Nuclear Medicine Communications 29(11): 1002-1005), or
c. Other mode of nuclear renal scanning, for example (see, for example, Uy, N. and Reidy, K. 2016. “Developmental Genetics and Congenital Anomalies of the Kidney and Urinary Tract,” Journal of Pediatric Genetics 5(1): 51-60; Chevalier, R. L. 2015. “Congenital Urinary Tract Obstruction: The Long View,” Advances in Chronic Kidney Disease 22(4): 312-319; Shukla, J. and Mittal, B. R. 2015. “Dimercaptosuccinic Acid: A Multifunctional Cost Effective Agent for Imaging and Therapy,” Indian Journal of Nuclear Medicine 30(4): 295-302; Reddy Gorla, A. K., Agrawal, K., Sood, A., Bhattacharya, A., and Mittal, B. R. 2014. “Differential Uptake of Te-99m [technetium-99 radioisotope] DMSA [dimercaptosuccinic acid] and Tc-99mEC [m] ethylene dicysteine] in Renal Tubular Disorders: Report of Two Cases and Review of the Literature,” Indian Journal of Nuclear Medicine 29(3): 160-162; Pulido, J. E., Furth, S. L., Zderic, S. A., Canning, D. A., and Tasian, G. E. 2014. “Renal Parenchymal Area and Risk of ESRD [end-stage renal disease] in Boys with Posterior Urethral Valves,” Clinical Journal of the American Society of Nephrology 9(3): 499-505; Metcalfe, P. D., Assmus, M., and Kiddoo, D. 2014. “Symptomatic Versus Asymptomatic Pyeloplasties: A Single Institution Review,” Canadian Urological Association Journal 8(11-12): 428-431; Hamed, M. A. 2014. “New Advances in Assessment of the Individual Renal Function in Chronic Unilateral Renal Obstruction Using Functional C T Compared to 99mTc-DTPA Renal Scan,” Nuclear Medicine Review. Central and Eastern Europe 2014 17(2): 59-64; Sarma, D., Barua, S. K., Rajeev, T. P., and Baruah, S. J. 2012. “Correlation between Differential Renal Function Estimation Using CT-based Functional Renal Parenchymal Volume and (99m) Te-DTPA [technetium-99 radioisotope-diethylene triamine pentaacetic acid] Renal Scan,” Indian Journal of Urology 28(4): 414- 417; Dissing, T. H., Eskild-Jensen, A., Mikkelsen, M. M., Pedersen, M., Frokiaer, J., Djurhuus, J. C., and Gordon, 1. 2008. “Normal Differential Renal Function Does Not Indicate a Normal Kidney after Partial Ureteropelvic Obstruction and Subsequent Relief in 2-week-old Piglets,” European Journal of Nuclear Medicine and Molecular Imaging 35(9): 1673-1680; Ortapamuk, H., Naldoken, S., Tekdogan, U. Y., Aslan, Y., and Atan, A. 2003. “Differential Renal Function in the Prediction of Recovery in Adult Obstructed Kidneys after Pyeloplasty,” Annals of Nuclear Medicine 17(8): 663-668). Another isotope used is mercaptoacetyltriglycine.

That takeoff at the ureters allows the kidneys to be separately monitored on a long-term basis without confinement to the clinic improves on this capability. Differential urinalysis is possible either by diversion from one of the kidneys, the other allowed to continue outflow through the lower tract so that only urine from the side to be monitored enters the collection bag, or by providing independent drainage lines leading to separate collection bags, isolating the urine emitted by either kidney. With an adjustable or intermittent embodiment, the admixture of urine from the contralateral ureter is avoided by retracting the chute on the contralateral ureter to allow urine to flow into the lower tract.

If a need for side to side follow-up kidney differential urinalysis is desired in a nonadjustable or prosthetic embodiment, the neoureters are each led to separate neoureter collection chambers and separate collection bags worn on the front or a side of either thigh. The output from each ureter is then implemented as a separate channel, if necessary, with independent rather than convergent chamber as shown in FIGS. 1, 4, and 5, with separate pressure actuated microswitches and neoureter convergence chamber impellers, which is a subminiature fluid driver similar to an axial box fan with blade guard, adapted to forcibly expel urine from the chamber if for any reason ureteric peristalsis fails to propel urine fully out of the line and into the collection bag so that the pressure in the chamber rises to a level portending ureteric reflux.

Ordinarily, no neoureter convergence chamber impeller, pressure actuated switch, button cell battery in the surface port, or wiring to connect the two are needed in the neoureter convergence chamber. This is because calyceal or pelvic and ureteral peristalsis forcibly drive the urine boluses down into the convergence chamber where the course of least resistance to continued outflow, with or without the added force of gravity, are the chamber outlet and line leading to the collection bag. The administration of a diuretic or obtaining diuresis by instructing the wearer to drink water can be used to stimulate intensified calyceal peristalsis and the jet effect (citations below) to overcome the resistance to outflow, or neurostimulation at the renal pelvis might be used to accomplish this.

However, diuretics and loop diuretics risk many potentially serious side effects, mostly by creating acid-base and electrolyte imbalances. These include hearing loss, lethargy, seizures, nausea, emesis, constipation, tachycardia, neuropsychological impairment, such as confusion, depression, anorexia, dizziness, and coma, pancreatitis, interstitial nephritis, skin rashes, thrombocytopenia, and myopathy, such as cramps, other myalgia, weakness, and paralysis (see, for example, Wehling, M. 2013. “Morbus Diureticus [diuretic disease] in the Elderly: Epidemic Overuse of a Widely Applied Group of Drugs,” Journal of the American Medical Directors Association (New York, New York) 14(6): 437-442; Palmer, B. F. 2011. “Metabolic Complications Associated with Use of Diuretics,” Seminars in Nephrology 31(6): 542-552; Sica, D. A., Carter, B., Cushman, W., and Hamm, L. 2011. “Thiazide and Loop Diuretics,” Journal of Clinical Hypertension (Greenwich, Connecticut) 13(9): 639-643; Sica, D. A. 2004. “Diuretic-related Side Effects: Development and Treatment,” Journal of Clinical Hypertension (Greenwich, Connecticut) 6(9): 532-540; Greenberg, A. 2000. “Diuretic Complications,” American Journal of the Medical Sciences 319(1): 10-24; Wilcox, C. S. 1999. “Metabolic and Adverse Effects of Diuretics,” Seminars in Nephrology 19(6): 557-568; Prichard, B. N., Owens, C. W., and Woolf, A. S. 1992. “Adverse Reactions to Diuretics,” European Heart Journal 13 Supplement G: 96-103; Weinberger, M. H. 1988. “Diuretics and Their Side Effects. Dilemma in the Treatment of Hypertension,” Hypertension (Dallas, Texas) 11(3 Part 2): 1116-1120).

To intensify peristalsis through electrical neuromodulation at the calyces responsive to a pressure actuated switch housed in the convergence chamber used or not used to actuate a chamber impeller is also possible but considered to needlessly place inordinate stress on normal peristaltic function at the pelvis. Intrinsic motility alone may not succeed when the wearer adopts certain postures, however, such as to lie on the side opposite the surface port, which can be discouraged by wearing the collection bag on the front rather than the outer side of the thigh. The surface port can be positioned midway along a short Pfannenstiel (bikini line incision, Kerr incision, Pfannenstiel-Kerr incision; pubic incision) however, the outlet hose must still veer toward the side of the collection bag. Most potential users will meet the requirement for peristaltic sufficiency, so that components to assist in the evacuation of the convergence chamber will not be not required.

However, ureteral peristalsis may be congenitally impaired, and can become temporarily impaired to remain impaired without treatment (see, for example, Hickling, D. R., Sun, T. T., and Wu, X. R. 2015. “Anatomy and Physiology of the Urinary Tract: Relation to Host Defense and Microbial Infection,” Microbiology Spectrum 3(4); Floyd, R. V., Upton, M., Hultgren, S., Wray, S., Burdyga. T. V., and Winstanley, C. 2012. “Escherichia coli-mediated Impairment of Ureteric Contractility is Uropathogenic E. [Escherichia] coli Specific,” Journal of Infectious Diseases 206(10): 1589-1596; Kopp. U. C., Cicha, M. Z., and Smith, L. A. 2002. “Endogenous Angiotensin Modulates PGE (2) [prostaglandin E2]-mediated Release of Substance P from Renal Mechanosensory Nerve Fibers,” American Journal of Physiology. Regulatory, Integrative, and Comparative Physiology 282(1): R19-R30; Kopp, U. C., Farley, D. M., Smith, L. A. and Knapp, H. R. 1995. “Essential Fatty Acid Deficiency Impairs the Responsiveness of Renal Pelvic Sensory Receptors,” American Journal of Physiology 268(1 Part 2): R164-R170).

Adjacent to the ureters, appendicitis and diverticulitis as well as “ . . . inflammatory, infectious, or malignant processes of the colon, appendix, oviducts, or ovaries” (http://emedicine.medscape.com/article/1949127-overview #showall) can impair ureteral peristalsis, tiny uroliths present or since passed can, as can ureteritis such as ureteritis cystica and urothelial neoplasms benign or malignant (Rathi. V., Agrawal. S., Bhatt. S., and Sharma, N. 2015. “Ureteral Dilatation with No Apparent Cause on Intravenous Urography: Normal or Abnormal? A Pilot Study,” Advances in Urology 2015:681836; Silverman, S. G., Leyendecker, J. R., and Amis, E. S. Jr. 2009. “What is the Current Role of C T Urography and M R Urography in the Evaluation of the Urinary Tract?,” Radiology 250(2): 309-323; Zelenko, N., Coll. D., Rosenfeld, A. T., and Smith, R. C. 2004. “Normal Ureter Size on Unenhanced Helical CT [computed tomography],” American Journal of Roentgenology 182(4): 1039-1041, available Online at http://www.ajrOnline.org/doi/pdf/10.2214/ajr. 182.4.1821039).

The causes of permanent ureteric impairment, often developmental or genetic as to metabolism and/or structure, which may prove resistant to treatment, as well as structural deficits following trauma, are equally if not more numerous (see, for example, Park, J., Goergen, C. J., HogenEsch, H., and Kim, C. H. 2016. “Chronically Elevated Levels of Short-chain Fatty Acids Induce T Cell-mediated Ureteritis and Hydronephrosis.” Journal of Immunology 196(5): 2388-2400; Canda, A. E., Dogan, H., Kandemir, O., Atmaca, A. F., Akbulu, Z., and Balbay, M. D. 2014. “Does Diabetes Affect the Distribution and Number of Interstitial Cells and Neuronal Tissue in the Ureter, Bladder, Prostate, and Urethra of Humans?,” Central European Journal of Urology 67(4): 366-374; Sanders, K. M., Ward, S. M., and Koh, S. D. 2014. “Interstitial Cells: Regulators of Smooth Muscle Function,” Physiological Reviews 94(3): 859-907; Kim, S. T., Ahn, S. Y., Swat, W., and Miner, J. H. 2014. “DLG1 [discs-large homolog 1, synapse-associated protein 97, SAP97 scaffold protein] Influences Distal Ureter Maturation via a Non-epithelial Cell Autonomous Mechanism Involving Reduced Retinoic Acid Signaling, Ret Expression, and Apoptosis,” Developmental Biology 390(2): 160-169; Osman, F., Romics, I., Nyirady, P., Monos, E., and Nadasy, G. L. 2009. “Ureteral Motility,” Acta Physiologica Hungarica 96(4): 407-426; Mahoney, Z. X., Sammut, B., Xavier, R. J., Cunningham, J., Go. G., and 4 others 2006. “Discs-large Homolog 1 Regulates Smooth Muscle Orientation in the Mouse Ureter,” Online, Proceedings of the National Academy of Sciences of the United States of America 103(52): 19872-19877; David, S. G., Cebrian, C., Vaughan, E. D. Jr., and Herzlinger, D. 2005. “c-kit and Ureteral Peristalsis,” Journal of Urology (Philadelphia, Pennsylvania) 173(1): 292-295; Kong, X. T., Deng. F. M., Hu, P., Liang, F. X., Zhou, G., and 7 others 2004. “Roles of Uroplakins in Plaque Formation, Umbrella Cell Enlargement, and Urinary Tract Diseases,” Journal of Cell Biology 167(6): 1195-1204; Deng. F. M., Liang, F. X., Tu, L., Resing, K. A., Hu, P., Supino, M., and 5 others 2002. “Uroplakin IIIb, a Urothelial Differentiation Marker, Dimerizes with Uroplakin Ib as an Early Step of Urothelial Plaque Assembly,” Journal of Cell Biology 159(4): 685-694; Hu, P., Deng. F. M., Liang, F. X., Hu, C. M., Auerbach, A. B., and 4 others 2000. “Ablation of Uroplakin III Gene Results in Small Urothelial Plaques, Urothelial Leakage. and Vesicoureteral Reflux,” Journal of Cell Biology 151(5): 961-972), and/or the wearer falls asleep in a position that minimizes or eliminates gravity as a contributing force to voiding, an automatic assist device averts reflux. When the need for an assist is temporary, the wearer simply inserts the button cell battery in the surface port as needed.

The apparatus described herein is able to eradicate the thrombus that ordinarily occludes catheters used to move blood by using the side-entry diversion jacket accessory or service channel to run an anticoagulant such as a low molecular weight or unfractionated heparin and/or vitamin K antagonists through the line. To avert the risk heparin can pose for an impaired kidney, protamine sulfate is kept available as a reversal agent. Ureteric obstruction due to distal ureteral ischemia can be addressed by diverting arterial blood to the ureter.

A larger jacket is placed about the source artery and the oxygenated blood is passed through a catheter and smaller jacket placed about the ureter. Accessory channels at both the takeoff or source artery and the input or destination jackets allow drugs to be targeted through the catheteric line for maintenance and the native lumina for therapy (Kumar, S., Ameli-Renani. S., Hakim, A., Jeon, J. H., Shrivastava, S., Patel, U. 2014. “Ureteral Obstruction following Renal Transplantation: Causes, Diagnosis and Management,” British Journal of Radiology 87(1044): 20140169).

With either a diversion or simple junction side-entry jacket, anastomotic stricture, which can arise in several different contexts along the digestive tract (see, for example, Carrodeguas, L., Szomstein, S., Zundel, N., Lo Menzo, E., and Rosenthal, R. 2006. “Gastrojejunal Anastomotic Strictures following Laparoscopic Roux-en-Y Gastric Bypass Surgery: Analysis of 1291 Patients,” Surgery for Obesity and Related Diseases 2(2): 92-97; Nguyen, N. T., Stevens, C. M., and Wolfe, B. M. 2003. “Incidence and Outcome of Anastomotic Stricture after Laparoscopic Gastric Bypass,” Journal of Gastrointestinal Surgery 7(8): 997-1003), urethra or ureter (see, for example, Raheem, O. A. and Buckley, J. C. 2014. “Adjunctive Maneuvers to Treat Urethral Stricture: A Review of the World Literature,” Translational Andrology and Urology 3(2): 170-178; Buckley, J. C. 2011. “Complications after Radical Prostatectomy: Anastomotic Stricture and Rectourethral Fistula,” Current Opinion in Urology 21(6): 461-464), so long as such procedures are continued, a gut-ureter junction as in an ileal conduit (see, for example, Dangle, P. P. and Abaza, R. 2012. “Robot-assisted Repair of Ureteroileal Anastomosis Strictures: Initial Cases and Literature Review,” Journal of Endourology 26(4): 372-376; Cheng. M., Looney, S. W., and Brown, J. A. 2011. “Ureteroileal Anastomotic Strictures after a Bricker Ileal Conduit: 50 Case Assessment of the Impact of Conversion from a Slit Incision to a “Shield Shaped” Ileotomy,” Canadian Journal of Urology 18(2): 5644-5649) will continue to pose problems of stricture, necessitating revision.

This is the more likely under various condition of disease, notably, tuberculosis of digestive and excretory tissue where anastomosis is not a factor (see, for example, Goel. A. and Dalela, D. 2008. “Options in the Management of Tuberculous Ureteric Stricture,” Indian Journal of Urology 2008 24(3): 376-381). With or without initial or subsequent endoscopic or balloon intervention, strictures anastomotic or due to disease can be treated through the direct targeting to the strictured segment of ameliorative if not curative drugs (see, for example, Boeree, M. J., Heinrich, N., Aarnoutse, R., Diacon, A. H., Dawson, R., Rehal. S., Kibiki, G. S., and 22 others 2017. “High-dose Rifampicin, Moxifloxacin, and SQ109 for Treating Tuberculosis: A Multi-arm, Multi-stage Randomised Controlled Trial,” Lancet. Infectious Diseases (London, England) 17(1): 39-49; Lalloo, U. G. and Ambaram, A. 2010. “New Antituberculous Drugs in Development,” Current HIV [human immunodeficiency virus]/AIDS [acquired immune deficiency syndrome] Reports 7(3): 143-151) through a simple junction, or if a ureter where diversion is needed, a urinary diversion jacket.

Automatic delivery directly to the site of the stricture from a port at the body surface, flat pectoral storage reservoir, and neoureter convergence chamber impeller, if necessary, overcomes patient noncompliance with drugs to be “ . . . given in combinations of four to six drugs for at least 6 months for drug-sensitive TB and up to 24 months for drug-resistant TB . . . ” (Sacksteder, K. A., Protopopova, M., Barry, C. E. 3rd, Andries, K., and Nacy, C. A. 2012. “Discovery and Development of SQ109: A New Antitubercular Drug with a Novel Mechanism of Action,” Future Microbiology 7(7): 823-837). Tuberculosis is cited in an exemplary sense, any infection for which an antimicrobial is available no less treatable thus. The term peristomal denotes the skin surrounding a stoma, while the term parastomal pertains to stomal herniation.

While less susceptible than a colorectal stoma, which must pass septic matter containing injurious enzymes and acids, an ileal conduit is susceptible to early and late postprocedural complications, to include renal deterioration and the need for dialysis (Kajmaković, B. M., Dzamić. Z. M., Pejcić. T., Milković, B., Aćimović, M., and Djokić, J. H. 2013. “Degree of Postoperative Renal Deterioration in Patients Who Have Undergone Ileal Conduit as a Form of Urinary Diversion in Relation to the Type of Insertion of the Ureter,” (in Serbian with English abstract at Pubmed), Acta Chirurgica lugoslavica 60(3): 17-24) ischemia, retraction, parastomal hernia, prolapse, stenosis, or stricture, necrosis, ulceration, fistulation, or fistulization, and herniation, predisposing to incarceration and strangulation of the gut, as well as posing a greater risk of infection, these complications more likely in patients with Crohn's disease (see, for example, Stewart, Z. E., Shaker, M., and Baxter, J. D. 2017. “Urinary Tract Infection Caused by Citrobacter koseri in a Patient with Spina Bifida, an Ileal Conduit and Renal Calculi Progressing to Peri-nephric Abscess and Empyema,” Urology Case Reports 11:22-24; Narang, S. K., Alam, N. N., Campain, N. J., Pathak, S., McGrath, J. S., Daniels, I. R., and Smart, N. J. 2016. “Parastomal Hernia Following Cystectomy and Ileal Conduit Urinary Diversion: A Systematic Review,” Hernia 2016 Dec. 26 (in press); Donahue, T. F. and Bochner, B. H. 2016. “Parastomal Hernias after Radical Cystectomy and Ileal Conduit Diversion,” Investigative and Clinical Urology 57(4): 240-248; Pan, T. Y. and Al-Sameraaii, A. 2016. “Use of the Memokath Urethral Stent in the Management of Ileal Conduit Stomal Stenosis,” International Journal of Surgery Case Reports 19:124-126; Muzzonigro, G. and Tombolini, F. 2015. “Ureterovaginal Fistulae,” (in Italian with English abstract at Pubmed), Urologia 82(1): 22-29; Liu, N. W., Hackney, J. T., Gellhaus, P. T., Monn. M. F., Masterson, T. A., and 4 others 2014. “Incidence and Risk Factors of Parastomal Hernia in Patients Undergoing Radical Cystectomy and Ileal Conduit Diversion” Journal of Urology (Philadelphia, Pennsylvania) 191(5): 1313-1318; Tarcoveanu, E., Vasilescu, A., Cotea, E., Vlad. N., and 3 others 2014. “Parastomal Hernias-Clinical Study of Therapeutic Strategies,” Chirurgia (Bucharest) 109(2): 179-184; Mohammad, W., Fode, M. M., and Azawi, N. H. 2014. “Treatment of Ureterovaginal Fistula Using a Memokath Stent,” Online, BMJ [British Medical Journal] Case Reports December 19 pii: ber2014207854; Kwiatt, M. and Kawata, M. 2013. “Avoidance and Management of Stomal Complications,” Clinics in Colon and Rectal Surgery 26(2): 112-121; Ng. K. L., Nawawi, O., Lim, B. K., Htun, T. H., Dublin, N., and Razack, A. H. 2013. “Antegrade Repositioning of Memokath [ureteral] Stent in Malignant Ureteroileal Anastomotic Stricture,” Asian Journal of Surgery November 6 pii: S1015-9584(13) 00102-00104; Wolff, B., Chartier-Kastler, E., Mozer, P., Haertig, A., Bitker, M. O., and Roupret, M. 2011. “Long-term Functional Outcomes after Ileal Ureter Substitution: A Single-center Experience,” Urology 78(3): 692-695; Zhang, Z. L., Liu, Z. W., Zhou, F. J., Li, Y. H., Xiong, Y. H., and 6 others 2010. “Modified Technique to Prevent Complications Related to Stoma and Ileoureteral Anastomosis in Patients Undergoing Ileal Conduit Diversion,” Urology 76(4): 996-1001; Armatys, S. A., Mellon, M. J., Beck, S. D., Koch, M. O., Foster, R. S., and Bihrle, R. 2009. “Use of ileum as Ureteral Replacement in Urological Reconstruction,” Journal of Urology (Philadelphia, Pennsylvania) 181(1): 177-181; Kann, B. R. 2008. “Early Stomal Complications,” Online, Clinics in Colon and Rectal Surgery (New York, New York) 21(1): 23-30; Husain, S. G. and Cataldo, T. E. 2008. “Late Stomal Complications,” Online, Clinics in Colon and Rectal Surgery (New York, New York) (New York, New York) 21(1): 31-40; Kim, J. T. and Kumar, R. R. 2006. “Reoperation for Stoma-related Complications,” Online, Clinics in Colon and Rectal Surgery 19(4): 207-212; Chung, B. I., Hamawy, K. J., Zinman, L. N., and Libertino, J. A. 2006. “The Use of Bowel for Ureteral Replacement for Complex Ureteral Reconstruction: Long-term Results,” Journal of Urology (Philadelphia, Pennsylvania) 175(1): 179-184; Simeone, C., Antonelli, A., Tonini, G., and Cunico, S. C. 2003. “Ileal Conduit and Urinary Stoma Complications,” (in Italian with English abstract at Pubmed), Archivio Italiano di Urologia, Andrologia 75(1): 6-9; Makela, J. T., Turku, P. H., and Laitinen, S. T. 1997. “Analysis of Late Stomal Complications Following Ostomy Surgery,” Annales Chirurgiae et Gynaecologie 86(4): 305-310). A body surface port made of synthetic material is critically less susceptible to such complications. The stoma is the cause of most complications following a relatively simple ureterocutaneostomy, or cutaneous ureterostomy. Additional references to early and late complications of stomata are cited below.

Rather than to replace a segment of the ureter with tubularized gut, another part of the digestive tract, oral mucosa, has been recruited to replace strictured (or otherwise no longer functional) segments along a ureter (Tsaturyan, A., Akopyan, K., Levonyan, A., and Tsaturyan, A. 2016. “Long Ureteric Stricture replacement by Buccal Mucosa Graft: An Armenian Experience Case Series Report,” Central European Journal of Urology 69(2): 217-220; Zhao, L. C., Yamaguchi, Y., Bryk, D. J., Adelstein, S. A., and Stifelman, M. D. 2015. “Robot-assisted Ureteral Reconstruction Using Buccal Mucosa,” Urology 86(3): 634-638). Given that degenerative metaplasia leading to malignancy is usually a long process so that cancer may not appear for 15 years or longer, the technique has not been in use long enough for sufficiently long-term follow-up (Kroepfl, D., Loewen, H., Klevecka, V., and Musch, M. 2010. “Treatment of Long Ureteric Strictures with Buccal Mucosal Grafts,” British Journal of Urology International 105(10): 1452-1455; Badawy, A. A., Abolyosr. A., Saleem, M. D., and Abuzeid, A. M. 2010. “Buccal Mucosa Graft for Ureteral Stricture Substitution: Initial Experience,” Urology 76(4): 971-975; Fichtner, J., Filipas. D., Fisch, M., Hohenfellner, R., and Thuroff. J. W. 2004. “Long-term Outcome of Ventral Buccal Mucosa Onlay Graft Urethroplasty for Urethral Stricture Repair,” Urology 64(4): 648-650); however, there is no reason to assume that exposure of this absorptive digestive tissue to urine would not exert the same degenerative effect over time as it exerts on the lining of the ileum (see for example, Rosenzweig, B., Mor, Y., Erlich, T., Laufer, M., Winkler, H., Kaver, I., Ramon, J., and Dotan, Z. A. 2016. “Urothelial-based Reconstructive Surgery for Upper- and Mid-ureteral Defects: Long-term Results,” Canadian Urological Association Journal 10(9-10): E290-E295). Such degeneration a slow process, the technique is best limited to the elderly and in then nonelderly, to the urethra, to which the means described herein do not apply.

Synthetic materials are not subject to complications such as ischemia, stricture, prolapse, retraction, swollenness for two months following stoma placement, necrosis, et cetera, where the means to deal with such conditions introduce additional complications. In addition to misappropriating a segment of gut, extending risk to another organ system, a stoma requires a significant breach of the abdominal musculature, critically weakening it, then puts the ileal segment through the breach, and once through, may roll the ileal segment back, where it often fails to heal with adequate adhesion to the skin and leaves a relatively large protrusion at the outside of the body, making it vulnerable to injury. Furthermore, long-term exposure of nonurinary tissue to urine invites metaplasia and malignancy.

The consequences are then, needless risk of injury and pronounced susceptibility to failure, whether present as retraction (see, for example, Siddique, K., Prud Homme, G., Samuel, N., Avil-Griffiths, K., and Offori, T. 2016. “A Novel Corrective Approach to Achieve Satisfactory Function of a ‘Sunk’ Colostomy,” Annals of the Royal College of Surgeons of England 98(5): e84-e87; Jayarajah, U., Samarasekara, A. M., and Samarasekera, D. N. 2016. “A Study of Long-term Complications Associated with Enteral Ostomy and Their Contributory Factors,” Online, BioMed Central Research Notes 9(1): 500; Schleicher, C., Senninger, N., Vowinkel. T., and Anthoni. C. 2010. “Stoma Prolapse and Stoma Retraction,” (in German with English abstract at Pubmed), Chirurg (Berlin, Germany) 81(11): 978-981; Husain, S. G. and Cataldo, T. E. 2008, Op cit.; Kim, J. T. and Kumar, R. R. 2006. “Reoperation for Stoma-related Complications,” Clinics in Colon and Rectal Surgery 19(4): 207-212), prolapse (see, for example, Monette, M. M., Harney, R. T., Morris, M. S., and Chu, D. I. 2016. “Local Repair of Stoma Prolapse: Case Report of an In Vivo Application of Linear Stapler Devices,” Annals of Medicine and Surgery (London) 11:32-35; Mehrotra, P., Yeh, J., Hall, T. R., Agopian, V. G., Farmer, D. G., and 3 others 2016. “Intestinal Perforation Following Ileoscopy through a Prolapsed Stoma in a Pediatric Intestinal Transplant Recipient with an Unrecognized Parastomal Hernia,” ACG [American College of Gastroenterology] Case Reports Journal 3(4): e123; Jayarajah, U., Samarasekara, A. M., and Samarasekera, D. N. 2016, Op cit.; Mohammed, O., West, M., and Chandrasekar, R. 2013. “Granulated Sugar to Reduce an Incarcerated Prolapsed Defunctioning Ileostomy,” Online, BMJ [British Medical Journal] Case Reports 2013. (London, England) pii: ber2012007565; Schleicher, C., Senninger, N., Vowinkel, T., and Anthoni. C. 2010. Op cit.; Husain, S. G. and Cataldo, T. E. 2008, Op cit.; Kim, J. T. and Kumar, R. R. 2006. Op cit.), or herniation (references above and below). “The most effective method of preventing a stoma complication is avoiding the creation of a stoma,” (Kim, J. T. and Kumar, R. R. 2006, Op cit.)

By contrast, the external ports to be described do not require a large breach through the abdominal musculature and inconspicuous, do not present a relatively large stoma which fails to adhere to the skin, making the integument mounting such a small port invulnerable to rupture due to the port, and a synthetic port can provide any, indeed more openings for incurrent and excurrent passage than any type of stoma. While the comparative failure rate of gastrointestinal and urinary stomata is undocumented, heavier loading would indicate that the former fail more often; nevertheless, urinary stomas fail often. For a synthetic port to fail structurally as hemiation, prolapse, or retraction is very much against the odds, and significantly, failure is a “ . . . prevalent problem and treatment can pose difficulties due to significant rates of recurrence and morbidities of the repair,” (Gillern, S. and Bleier, J. I. 2014. “Parastomal Hernia Repair and Reinforcement: The Role of Biologic and Synthetic Materials,” Online, Clinics in Colon and Rectal Surgery (New York, New York) 27(4): 162-171).

By contrast, a surface port made of a suitable polymeric material, such as gear hardness grade nylon (Higgins, D. M., Basaraba. R. J., Hohnbaum, A. C., Lee, E. J., Grainger, D. W., and Gonzalez-Juarrero. M. 2009, Op cit.; Chegini, N., von Fraunhofer, J. A., Hay, D. L., Stone, I. K., and Masterson B. J. 1987. “The Use of Nylon Pouches to Prevent Cellular Attachment to Implanted Materials,” Biomaterials 8(4): 315-319) or polyvinyl chloride (Sharma, R. and Vasudevan, P. 1982. “Biocompatibility Screening of Poly(Vinyl Chloride) Implants,” Biomaterials 32): 109-112), especially with easy access to instill a germicide, is less susceptible to infection and in an adult, requires a small breach of 3.0 millimeters for the excurrent or effluent opening to connect the outlet hose leading to the urine collection bag, with 2.0 millimeter holes for as many accessory channels as needed, 2.0 millimeter spaces to mount push/pull control cable knobs if needed, and a small compartment for a neoureter convergence chamber impeller battery if present. Overall, the port has an outer diameter of about 8.0 millimeters when the accessory channel or channels are above the skin and 5.0 millimeters when the accessory channel or channels are subdermal.

Neither prevention through the placement of prophylactic mesh or fascial fixation during the procedure to construct the ileal conduit dependably eliminates parastomal herniation (Pianka, F., Probst. P.1., Keller, A. V., Saure, D., Grummich, K., Buchler, M. W., and Diener, M. K. 2017. “Prophylactic Mesh Placement for the PREvention of paraSTOmal Hernias: The PRESTO Systematic Review and Meta-analysis,” Online, PLoS [Public Library of Science] One 12(2): e0171548; Wang, S., Wang, W., Zhu, B., Song, G., and Jiang, C. 2016. “Efficacy of Prophylactic Mesh in End- Colostomy Construction: A Systematic Review and Meta-analysis of Randomized Controlled Trials,” World Journal of Surgery 40(10): 2528-2536; Donahue. T. F., Cha, E. K., and Bochner, B. H. 2016. “Rationale and Early Experience with Prophylactic Placement of Mesh to Prevent Parastomal Hernia Formation after Ileal Conduit Urinary Diversion and Cystectomy for Bladder Cancer,” Current Urology Reports 17(2): 9; Styrke, J., Johansson, M., Granasen, G., and Israelsson, L. 2015. “Parastomal Hernia after Ileal Conduit with a Prophylactic Mesh: A 10 Year Consecutive Case Series,” Scandinavian Journal of Urology 49(4): 308-312; DeAsis, F. J., Linn, J. G., Lapin, B., Denham, W.2, Carbray, J. M., and Ujiki, M. B. 2015. “Modified Laparoscopic Sugarbaker Repair Decreases Recurrence Rates of Parastomal Hemia,” Surgery 158(4): 954-961; Pisters, A. L., Kamat, A. M., Wei, W., Leibovici, D., Liu, J., Grossman, H. B., and Butler, C. E. 2014. “Anterior Fascial Fixation Does Not Reduce the Parastomal Hernia Rate after Radical Cystectomy and Ileal Conduit,” Urology 83(6): 1427-1431; Hotouras, A., Murphy, J., Thaha, M., and Chan, C. L. 2013. “The Persistent Challenge of Parastomal Herniation: A Review of the Literature and Future Developments,” Colorectal Disease 15(5): e202-14). Moreover, herniation may be repetitive, resulting in multiple procedures to correct the problem (Wada, T., Kawada, K., Hasegawa, S., and Sakai, Y. 2016. “Laparoscopic Repair for Recurrent Parastomal Hernia of an End Stoma Using the Sandwich Technique while Preserving an Ileal Conduit: A Case Report,” International Journal of Surgery Case Reports 25:75-78; Chang, D. T., Thyer, I. A., Larkin, J. O., Wallace, M. H., and Hayne, D. 2014. “First Report of the Stapled Mesh Stoma Reinforcement Technique in a Urologic Context,” Case Reports in Urology 2014:294304).

Unlike simple junction side-entry jackets as described in U.S. Pat. No. 11,759,186, entitled Ductus Side-entry Jackets and Prosthetic Disorder Response Systems, which are cylindrical, to allow sufficient radius for the diversion chute and mechanism used to deploy—and in an intermittent embodiment, to retract—the chute, a blood or urine diversion side-entry jacket is elongated on the side of the chute. Seen from above, it can therefore be described as oblong or ‘coccobacillar,’ that is, substantially rectangular with rounded corners, the hole for the ductus on the unprojected side off-center in focus.

Diversion jackets are therefore placed to encircle the vessel or ureter at the rotational angle that least if at all encroaches upon neighboring tissue. However, due to the small size of the jackets used for ureteral takeoff, at about 1.0 centimeter in length and 0.5 centimeter in height, encroachment should seldom if ever pose a problem. These dimensions can be reduced if necessary, such as for application in a neonate or infant, but are preferable to the minimum to expedite manipulation during placement.

Diversion jackets differ from side-entry jackets generally in that flow through the mainline is excurrent for drainage rather than incurrent, usually to target a drug into the encircled ductus. If used to divert arterial flow, the excurrent flow is continuous with peaks in pulsatile pressure, necessitating a water jacket to prevent extravasation during placement. However, if used to divert urine, lengthy interruptions in outward flow allow the unconflicted incurrent, or inflowing, delivery of medication between outflows, without the need for a water jacket. With either the continuous outflow of a vessel or the intermittent outflow of a ureter, to prevent a drug or other agent intended for the portion of the ductus beneath (distal, inferior, or caudal to) the jacket from being flushed out through the mainline, the accessory or service channel or channels must continue entirely into the native lumen.

This is accomplished by continuing the accessory channel as a groove with open bottom running along the underside of the diversion chute. Both fixed and intermittent use embodiments do, however, incorporate at least the one accessory channel running along the underside of the diversion chute to allow medication to be introduced into any portion of the lower tract remaining that may require treatment or be potentially recoverable. Drugs or agents to target a portion of the ductus superior or craniad (cephalad) to the diversion jacket which are not to be administered systemically are injected into the surface port for entry into the ductus through another jacket positioned in the higher location. In an intermittent use embodiment, retraction of the diversion chute allows additional accessory channels to release drugs or other agents into the native lumen.

Thus, except in an embodiment with diversion chute fixed in position for use as a prosthesis when the lower urinary tract or a significant portion thereof is missing, an exceptional accessory channel needed in addition to that connected to and feeding through the diversion chute is a simple tube or pipe bonded to the internal surface of the mainline and extended into the native lumen to empty in the direction of flow. An exception to the generality that a prosthesis is always nonadjustable pertains when there will be an ongoing need for differential urinalysis of the urine produced by either kidney, and the urinalysis must apply to samples taken over more time than the wearer must remain in the clinic. In that case, an adjustable or intermittent embodiment must be used; however, taking samples by passing a catheter through an accessory channel and into the trepan end-piece allows differential spot checks to be made with a nonadjustable embodiment.

The distal ends of such an accessory channel are tucked inside the trepan section during incision and pushed out into the lumen before attaching the mainline to which the trepan section is connected and closing. The need for one or more additional accessory channels pertains only to an intermittent use embodiment, often for treatment of the bladder, and only in the unusual circumstance that drugs for treating the tract below the jacket to be administered locally, or topically, must be kept separate. An intermittent use embodiment placed to bypass an intractably overactive bladder, for example, poses such a potential (see, for example, Srikrishna, S., Robinson, D., and Cardozo, L. 2014. “Important Drug-drug Interactions for Treatments that Target Overactive Bladder Syndrome,” International Urogynecology Journal 25(6): 715-720).

For this reason, with arterial diversion, a drug or drugs can be delivered without regard to the phase in pressure variation of the excurrent flow. A urinary diversion system is distinct in requiring that to treat the proximal ureter, it be possible to deliver drugs through the mainline, while to treat the distal ureter, it be possible to deliver drugs through an accessory channel, or sideline. Because the accessory channel continues into the native lumen in the direction of flow, drugs can be delivered regardless of whether micturition is in progress. However, drugs not to be mixed and diluted with urine, even sterile, are meted out during nonflow intervals.

Where the urine is contaminated and the drug should be administered as sterile, both main and sidelines allow lavage, an initial wash usable to furnish a diagnostic test sample. The timed administration of drugs or system maintenance agents can be accomplished manually by a competent patient or automatically by a system that includes the required sensor and microprocessor of timing control module. When urinary diversion is the only object, no timing module or microprocessor is needed, forced ejection of urine from the neoureter convergence, or confluence, chamber—which is required in all but an embodiment meant for use exclusively during the daytime, is executed by a pressure actuated switch in the chamber which is attached to and activates the neoureter convergence chamber impeller.

In unilateral application where only one kidney is affected and it is desired to independently analyze the effluent on that side so that a transureterouretostomy, for example, is not applicable, or in bilaterally separated application where each ureter is to empty through its own surface port into its respective collection bag, a confluence chamber is omitted, each side provided with its own independent channel whereby an evacuation assist impeller is bilateral without confluence, with a button cell battery in each surface port for power. In adjustable embodiments, the incorporation of a third push/pull cable control knob in the mons pubis port with cutaneous openings for the accessory channels allows control of a synthetic neobladder with stopper-flap for continence, thus allowing voiding in a reasonably normal manner.

A strain gauge and sensor and miniature rotor with eccentric load as mentioned above signal the need to void. Unless the need to empty the neobladder is frequent, the increase in storage capacity of the port battery needed to power both the neoureter convergence chamber elimination assist impeller and the motor used to signal that the neobladder is full is negligible. A second port to allow separate power for each function has diagnostic advantage in confirming that the source of inoperability of either is not due to low power. If the frequency is high, a larger battery is used. If causes discomfort, a second battery can be positioned in a second surface port positioned elsewhere, to include the contralateral side. When a future need for such a neobladder is anticipated, a surface port with button cell battery compartment is placed at the outset.

For use in an arterial shunt, the blood flow diversion chutes of the vascular servovalves shown in FIGS. 7 and 8, for example, would be positioned within the lumen of the mainline. If upon extraction the plug of tissue removed to create an ostium catches, a fine guidewire with hooked tip is used to remove it from the line. With a ureter, the plug will usually be flushed out through the line spontaneously by the progressive buildup in pressure. For the plug to resist removal to the extent that it induces hydroureteronephrosis (ureterohydronephrosis) or hydronephrosis is unlikely but remediable by retrieval with a fine gauge hook tipped guidewire passed through the urine diversion effluent or outlet line, or mainline.

When no opening in the port need be open to the outside, the openings are all subdermal, that is, covered over by dermis. Such a port is typically placed in the pectoral region for injection of drugs or line maintenance agents by fine hypodermic needle or jet injection. A port which provides a urine diversion outlet will typically be positioned as is the stoma of an ileal conduit, at the surface of the skin in the lower abdomen. When only one of the openings into the port must remain open to the outside, as is the case with a urine diversion outlet or an opening to pass a cabled device on a frequent basis, the opening is mounted on the outside of the skin, other openings not requiring to be surface mounted then kept beneath the skin.

Openings into the port which must serve for fine hypodermic needle or jet injection so frequently that the skin would be injured and not allowed to heal are kept above the skin; however, if multiple openings are led into the same drug delivery line thus minimizing the trauma to the skin, then these openings are positioned subdermally. While open access through the dermis requires a permanent opening, U.S. Pat. No. 11,759,186 entitled Ductus Side-entry Jackets and Prosthetic Disorder Response Systems, describes a multiple opening port for mounting on the skin. The port, shown in FIGS. 27 and 28, is described in the specification. The differently configured body surface ports herein are shom in FIGS. 26A thru 26C. Tissue adhesion using synthetic tubing is approximated when the outer surface of the tubing is configured to encourage tissue ingrowth.

By contrast, the presupposition that a stoma provides serosa to fascia tissue adhesion or fusion between the ileum and the surrounding hypodermal fascia to form a permanent bond to seal out the environment is mistaken: given enough time, the faulty junction will eventually give rise to a parastomal hernia, which can cause discomfort and disfigurement necessitating use of a custom girdle or abdominal support to further degrade the quality of life (see, for example, Donahue, T. F., Cha, E. K., and Bochner, B. H. 2016. “Rationale and Early Experience with Prophylactic Placement of Mesh to Prevent Parastomal Hernia Formation after Ileal Conduit Urinary Diversion and Cystectomy for Bladder Cancer,” Current Urology Reports 17(2): 9; Styrke, J., Johansson, M., Granåsen, G., and Israelsson, L. 2015. “Parastomal Hernia after Ileal Conduit with a Prophylactic Mesh: A 10 Year Consecutive Case Series,” Scandinavian Journal of Urology 49(4): 308-312).

More serious parastomal herniation can lead to incarceration, strangulation, and considerable pain, necessitating surgical revision, or repair (see, for example, Donahue, T. F. and Bochner, B. H. 2016. “Parastomal Hernias after Radical Cystectomy and Ileal Conduit Diversion,” Investigative and Clinical Urology 57(4): 240-248; Narang, S. K., Alam, N. N., Campain, N. J., Pathak, S., McGrath, J. S., Daniels, I. R., and Smart, N. J. 2016. “Parastomal Hernia following Cystectomy and Ileal Conduit Urinary Diversion: A Systematic Review,” Hernia [Online] December 26; Liu, N. W., Hackney, J. T., Young, M. J. and Weston, P. M. 2015. “Obstruction of an Ileal Urinary Conduit in an Incarcerated Right Inguinal Hernia,” Annals of the Royal College of Surgeons of England 97(3): e46; Liu, N. W., Hackney, J. T., Gellhaus, P. T., Monn, M. F., Masterson, T. A., and 4 others 2014. “Incidence and Risk Factors of Parastomal Hernia in Patients Undergoing Radical Cystectomy and Ileal Conduit Diversion,” Journal of Urology (Philadelphia, Pennsylvania) 191(5): 1313-1318; Pisters, A. L., Kamat, A. M., Wei, W., Leibovici, D., Liu, J., Grossman, H. B., and Butler, C. E. 2014. “Anterior Fascial Fixation Does Not Reduce the Parastomal Hernia Rate after Radical Cystectomy and Ileal Conduit,” Urology 83(6): 1427-1431; Donahue, T. F., Bochner, B. H., Sfakianos, J. P., Kent, M., Bemstein, M., Hilton, W. M., and 4 others 2014. “Risk Factors for the Development of Parastomal Hernia after Radical Cystectomy,” Journal of Urology (Philadelphia, Pennsylvania) 191(6): 1708-1713; Heo, S. C., Oh, H. K., Song, Y. S., Seo, M. S., Choe, E. K., Ryoo, S., AND Park, K. J. 2011. “Surgical Treatment of a Parastomal Hernia,” Journal of the Korean Society of Coloproctology 27(4): 174-179; Izumi, K., Kono, M., Kato, H., Mihara, S., Tsukahara, K., and Namiki, M. 2007. “Incarcerated Intestinal Hernia in a Hernia Sac of Reversed Ileal Conduit Wall Protruding through the Stoma,” International Journal of Urology 2007 14(6): 561-562; Yoshida, T., Ujike, T., Uemura, M., Nin, M., Nishimura, K., Miyoshi, S., Noro, H., and Nezu, R. 2007. “Parastomal Hernia with Incarcerated Necrosis at Ascending Colon 18 Years after the Construction of Ileal Conduit: A Case Report,” (in Japanese with English abstract at Pubmed), Hinyokika Kiyo [Acta Urologica Japonica] 53(3): 187-189).

Other complications associated with ileal conduit stomas made of gut as opposed to synthetics are prolapse, retraction, and stenosis (see, for example, Szymanski, K. M., St-Cyr. D., Alam, T., and Kassouf. W. 2010. “External Stoma and Peristomal Complications following Radical Cystectomy and Ileal Conduit Diversion: A Systematic Review,” Ostomy Wound Management 56(1): 28-35: Kouba, E., Sands, M., Lentz, A., Wallen, E., and Pruthi, R. S. 2007. “Incidence and Risk Factors of Stomal Complications in Patients Undergoing Cystectomy with Ileal Conduit Urinary Diversion for Bladder Cancer,” Urology 178(3 Part 1): 950-954; Wood, D. N., Allen, S. E., Hussain, M., Greenwell, T. J., and Shah, P. J. 2004. “Stomal Complications of Ileal Conduits are Significantly Higher when Formed in Women with Intractable Urinary Incontinence,” Journal of Urology (Philadelphia, Pennsylvania) 172(6 Part 1): 2300-2303). By contrast, a properly designed surface port made of synthetic material to replace a stoma constructed of native tissue is substantially if not entirely ‘immune’ to such complications.

Moreover, even when attention is paid to maintaining the stoma so that peristomal complications such as skin irritation and lesioning, or skin breakdown, to include (Marshall, C., Woodmansey, S., and Lyon, C. C. 2017. “Peristomal Psoriasis,” Clinical and Experimental Dermatology February 13 [in press]; Szymanski, K. M., St-Cyr. D., Alam, T., anf Kassouf, W. 2010. Op cit.: “ . . . irritant contact dermatitis, pseudoverrucous lesions, alkaline encrustations), mechanical injury (pressure ulcers, skin stripping injuries, mucocutaneous separation), infection (candidiasis, folliculitis), immunologie disorders (allergic contact dermatitis), and disease-related lesions (varices, pyoderma gangrenosum, malignancy . . . ”) do not occur (see, for example, Boyles, A. and Hunt, S. 2016. “Care and Management of a Stoma: Maintaining Peristomal Skin Health,” British Journal of Nursing 25(17): S14-S21; Rudoni, C. 2011. “Peristomal Skin Irritation and the Use of a Silicone- based Barrier Film,” British Journal of Nursing 20(16): S12, S14, S16; Omura, Y., Yamabe, M., and Anazawa, S. 2010. “Peristomal Skin Disorders in Patients with Intestinal and Urinary Ostomies: Influence of Adhesive Forces of Various Hydrocolloid Wafer Skin Barriers,” Journal of Wound, Ostomy, and Continence Nursing 37(3): 289-298; Calum C Lyon, C. C. and Smith, A. (eds.) 2010. Abdominal Stomas and their Skin Disorders: An Atlas of Diagnosis and Management, London, England: Informa Healthcare), a stoma, whether the terminus of an ileal conduit, an ileostomy, or a colostomy, is subject to several serious complications, both early (before 30 days) and late (after 30 days)(see, for example, Carlsson, E., Fingren, J., Hallen, A. M., Petersen, C., and Lindholm, E. 2016. “The Prevalence of Ostomy-related Complications 1 Year after Ostomy Surgery: A Prospective, Descriptive, Clinical Study,” Ostomy Wound Management 62(10): 34-48; Sheetz, K. H., Waits. S. A., Krell, R. W., Moris, A. M., Englesbe, M. J., Mullard, A., Campbell, D. A., and Hendren, S. 2014. “Complication Rates of Ostomy Surgery are High and Vary Significantly between Hospitals,” Diseases of the Colon and Recha 57(5): 632-637; Kwiatt, M. and Kawata, M. 2013. Op cit.; Bafford, A. C. and Irani, J. L. 2013. “Management and Complications of Stomas,” Surgical Clinics of North America 93(1): 145-166; Salvadalena, G. D. 2013. “The Incidence of Stoma and Peristomal Complications during the First 3 Months after Ostomy Creation,” Journal of Wound, Ostomy, and Continence Nursing 40(4): 400-406; Ratliff. C. R. 2010. “Early Peristomal Skin Complications Reported by WOC [wound, ostomy, and continence] Nurses,” Journal of Wound, Ostomy, and Continence Nurses 37(5): 505-510; Shabbir, J. and Britton, D. C. 2010. “Stoma Complications: A Literature Overview,” Colorectal Disease 12(10): 958-964; Nybaek, H1. and Jemnec, G. B. 2010. “Skin Problems in Stoma Patients,” Journal of the European Academy of Dermatology and Venereology 24(3): 249-257; Nybaek, H., Bang Knudsen, D., Norgaard Laursen. T., Karlsmark, T., and Jemec, G. B. 2009. “Skin Problems in Ostomy Patients: A Case-control Study of Risk Factors,” Av.Acta Dermato-venereologica 89(1): 64-67; Kann, B. R. 2008, Op cit.; Ratliff, C. R., Scarano, K. A., Donovan, A. M., and Colwell, J. C. 2005. “Descriptive Study of Peristomal Complications,” Journal of Wound, Ostomy, and Continence Nursing 32(1): 33-37).

Proportional in size to the ductus encircled, a ductus side-entry jacket for placement along a ureter, typically 1.0 centimeter in its longer outer diameter, is likewise millimetric in dimensions. The need for surgical reentry and replacement depends upon the relative diameter or caliber of the ductus upon placement and the later date; a jacket placed in an infant eventually will require replacement. However, because the accessory or service channel allows almost any other cause of fouling to be eradicated, replacement should not be necessary for years. An accessory channel leading into the lumen of the jacket afford access for maintenance of the jacket and lower urinary tract, while one leading into the foam lining the jacket allows the direct delivery to the foam ductus interface should an adverse tissue reaction beyond the normal foreign body response materialize.

The incorporation of a luminal contents tap, or takeoff, jacket diversion chute (takeoff chute, diversion chute) into a ureteral side-entry jacket to allow urinary diversion represents but a minor adaptation of such a jacket, which generally is used to establish a continuum between a synthetic and a native lumen. More widely applied without a takeoff chute, a ductus side-entry jacket can be used to encircle any type ductus to allow direct communication with a port at the body surface. This allows, for example, the directly targeted infusion of a drug into a blood vessel. While for urinary diversion, the subdermally or subcutaneously positioned port incorporates a hole opened to the outside to allow the diversion of urine into an external collection bag, the need for an opening to the outside is exceptional and such a port for injecting or infusing medication through accessory channels will be fully subdermal, which is to say, fully internal, fully implanted.

When systemic drugs prove unsatisfactory, once the port and line have been sterilized by lavage with wash water containing an antimicrobial, drugs can be targeted into the tract lying above (cephalad, craniad, superior to) the level of the jacket by injection through the urine outlet opening in the surface port. When the tract lying above the diversion jacket requires intensive drug therapy, a second jacket is positioned higher up on the ureter. Retraction of the diversion chute in the lower diversion jacket allows drugs delivered through the upper jacket to wash down through the lower tract. When the diversion chute must remain advanced to close off the ureter and divert urine into the outlet line, a channel running along the center of the underside of the diversion chute serves as a passageway to allow the delivery of drugs directly into the tract beneath (caudad, inferior to) to the level of the diversion jacket.

The chute is made of soft, shape-compliant material such as a Tygon® (Saint-Gobain, La Defense, Courbevoie, France), a more stiffly formulated tissue filler, or similar material in a composite or laminated to achieve the mechanical properties essential for the chute to adopt the contour without injuring the ureter. In an intermittent or adjustable embodiment, this is essential first, because an overhanging rim (awning, upper eyelid, palpebrum, palpebrarum) along the upper edge of the accessory channel opening into the ureteral lumen is needed to prevent retrograde or back-flow of medication or other agents up the ureter when the wearer is decubitus (recumbent) with the chute retracted. By contrast, in a nonadjustable or fixed embodiment for use as a prosthesis, such an overhang is unnecessary, because the chute is fixed in the extended position. With the chute in a fixed embodiment and in an intermittent (adjustable) embodiment when deployed (extended, advanced), a good seal requires that the surrounding edge of the chute slightly protrude into the wall of the lumen.

The edge is therefore made to fold upwards without gouging the urothelium, the overhanging rim in the adjustable embodiment folding flat when placed in abutment against the wall and therefore not protruding more than the rest of the edge. Were a stiff material used to make the chute, with either a fixed or an adjustable embodiment while the chute is advanced, the surrounding edge of the chute and this overhang would protrude into, and inevitably incise, the ureteral wall. Were the chute not made of soft shape-compliant materials with thin upturned, feathered and rolled edge bias molded to fold upward rather than protrude into the urothelium, erosion and device failure would soon result. In a fixed embodiment for use as a prosthesis, the chute once deployed is fixed in extension beyond the leading edge of the trepan.

Avulsion of a ureter at both pelvic and cystic ends sometimes occurs as a rare complication of ureteroscopy or accidental trauma; however, even in an adult with a wall thickness of only 1.0 millimeter,—a structure which includes an outer longitudinal and an inner circular layer of smooth muscle to support peristalsis should be great enough in tensile strength that this would follow the use of excessive or misdirected force and/or the ureter includes a segment rendered malacotic by disease or injury following the passage of a stone. Once reattached with microsutures, the ureter can be fitted with a diversion jacket during the same procedure. At 2.0 millimeters in width and length, the chutes in both adjustable and nonadjustable embodiments sized for the average adult exceed the internal diameter of the native lumen even at its peak caliber under diuresis during peristaltic bolus expulsion.

In FIGS. 2, 5, 7, 8, and 10A, part number 30 is an obturator fused to the distal tip of diversion chute 18. Its purpose is to completely close off outflow through sidestem 19 when diversion chute 18 is fully retracted thus restricting outflow to continuation through the substrate ureter. In an adjustable embodiment, the complete obturation of the sidestem entry outlet prevents the entry of urine into the device when the chute is retracted so that outflow is limited to the native ureter. However, should obturator 30 not fully seal off outflow through sidestem 19, the consequence will be leakage passing into the lower tract which the wearer will expel during voiding.

In a prosthesis, however, where the lower tract is missing or dysfunctional, diversion is constant, eliminating the need for a side-entry obturator but necessitating the prevention of any leakage past the chute over time that would collect in the pelvis as would pose the risk of sepsis, or if the free end of the ureter is closed off, then in the irregular space between the underside of the chute and the ureteral terminus to pose a potential threat of infection. In both adjustable and nonadjustable embodiments, the thinned out or feathered edges of the chute, molded to ride up against the lumen wall, and the compliance under compression of the urothelium, the ureteral lumen should be completely cut off from the lumen above and thus unable to leak urine past the chute. In a nonadjustable embodiment, that is, a prosthesis, diversion is not through valves but rather fixed side-entry diversion jackets.

So that this compliance does not allow any portion of the obturators, which must remain flush against and cover over the side-entry holes, to be pushed into the entry-holes creating a breach and partial outflow, the obturators must exceed the side-entry holes in area. The obturators must pass through the side-entry holes only when the device is initially placed, and if applicable, when removed. To allow the obturators to pass through the side-entry holes and not catch on the trepan or injure the urothelium bounding the trepan if removed, the obturators must be dimensionally changeable. This is accomplished by making the obturators of a shape-memory polymer. The obturators are circular and fastened at their centers to the distal tips of the diversion chutes.

At body temperature, the obturators are circular and pliant as to lay flush against without injuring the urothelium both when advanced and retracted. So that the obturators will not be damaged when flush against the urothelium with the chutes retracted, the trepans are slightly recessed. The diversion chute is retracted to allow passage of the side-entry plug when manually or vacuum-extracted during placement. Once within the lumen, the operator deploys the side-entry hole obturator and advances the chute until it presses against the opposite wall of the ureter in rounded and flush relation thereto. Upon retraction, the obturator is brought flush against the near wall of the ureter with the trepan receded by 0.25 millimeters relative to the internal edge of the side-entry hole.

This leaves the near wall of the ureteral lumen surrounding the side-entry hole under negligible compression when the obturator is retracted to allow voiding without diversion. Since the urothelium is 1. Adapted to compression during and to the degree posed by peristalsis (which is less than sphincteric) when passing a bolus of urine, 2. The degree of compression is less than it is during peristalsis. 3. The duration of obturation is limited to time performing or sleeping, and 4. The lining of the side-entry jacket allows the unimpeded flow of oxygenated blood through the fine vessels of the adventitia without compressing the fine nerves thereof, the urothelium should remain unaffected. In a prosthesis, to preserve what peristaltic function remains, the jacket is placed as distad or caudally as possible along the ureter. If the distal tract remains, the diversion chute continues the accessory channel to its tip, allowing the direct delivery to the lower tract of medication. Rather than to make an additional embodiment merely to omit continuation of the accessory channel through the chute, the channel is simply not used.

In a prosthesis, that is a nonadjustable permanent embodiment, with the jackets situated near to the distal termini of the ureters, no passage and collection of urine past the diversion chute to leak into the pelvic cavity, while unlikely, can be tolerated. This is prevented by transecting the ureter about an eighth of an inch below the chute, injecting the irregular spaces beneath the diversion chute with a permanent injectable tissue filler, pinching the open end of the ureter closed with surgical cyanoacrylate cement, and suturing the ureter closed. The distinct improbability of leakage and little if any consequence were it to occur mean that a temporary nonadjustable embodiment placed to divert urine away from and deliver medication to expedite healing of the distal tract following surgery, for example, requires no ureteral stump end-plugs.

A tissue filler used to make the chute is not jellylike as are those used in plastic surgery but just firm enough to deform as required to maintain obturation of the ureteral lumen. In addition to the shape conformity needed, the material requirements are stringent, in that the material must be invulnerable to hydrolysis and enzymatic or microbial breakdown while provoking no more than a mild and transient adverse tissue reaction In conventional use, hydrogel-based tissue fillers can cause several serious complications (see, for example, Woodward, J., Khan, T., and Martin, J. 2015. “Facial Filler Complications,” Facial Plastic Surgery Clinics of North America 23(4): 447-458; Lee, S. K., Kim, S. M., Cho, S. H., Lee, J. D., and Kim, H. S. 2015. “Adverse Reactions to Injectable Soft Tissue Fillers: Memorable Cases and Their Clinico-pathological Overview,” Journal of Cosmetic and Laser Therapy 17(2): 102-108; El-Khalawany, M., Fawzy, S., Saied, A., Al Said, M., Amer. A., and Eassa, B. 2015. “Dermal Filler Complications: A Clinicopathologic Study with a Spectrum of Histologic Reaction Patterns,” Annals of Diagnostic Pathology 19(1): 10-15; Souza Felix Bravo, B., Klotz de Almeida Balassiano, L., Roos Mariano da Rocha, C., Barbosa de Sousa Padilha, C., Martinezt Torrado, C., Teixeira da Silva, R., and Carlos Regazzi Avelleira, J. 2015. “Delayed-type Necrosis after Soft-tissue Augmentation with Hyaluronic Acid,” Journal of Clinical and Aesthetic Dermatology 8(12): 42-47). However, here the chute is made of less fluid material and is so positioned that these complications cannot arise.

Hyaluronic acid and fillers containing nonsynthetic components, for example, are more likely to arouse an immune or foreign body reaction and are not recommended. Fillers are used for cosmetic facial surgery where the filler can spread away, or migrate, from the injection site. Since for the present purpose the hardness-adjusted filler is constrained in position within the ureteral lumen, this detraction pertaining to fillers does not apply. Depending upon the medical context, medical-grade liquid injectable silicone and polymethyl methacrylate would appear to pose few if any problems for the use stated (Joseph, J. H. 2015. “The Case for Synthetic Injectables,” Facial Plastic Surgery Clinics of North America 23(4): 433-445). Additionally, in an adjustable embodiment, complete obturation would prevent the direct delivery of drugs to treat the tract inferior to the jacket.

The material of the chute must not be susceptible to biodegradation, or breakdown due to enzymatic action, acidity, or hydrolysis by almost constant exposure to urine, or by bacteria in the urine or in the blood supply to the ureter in disease (Thomas, B. S., Okamoto, K., Bankowski, M. J., and Seto, T. B. 2013. “A Lethal Case of Pseudomonas putida Bacteremia Due to Soft Tissue Infection,” Infectious Diseases in Clinical Practice (Baltimore). 21(3): 147-213). Depolymerization of certain polymers yields hazardous monomers.

Subject to breakdown by certain pathogenic bacteria such as Pseudomonas putida, once formulated to obtain the required hardness, cross-linked polyacrylamide hydrogel (the soft tissue filler is sold as Aquamide and Bulkamide, Contura International, Montreux, Switzerland and Soborg, Denmark), for example, is unacceptable where infection from this and similar pathogens exists (Yu, F., Fu. R., Xie. Y., and Chen, W. 2015. “Isolation and Characterization of Polyacrylamide-degrading Bacteria from Dewatered Sludge,” International Journal of Environmental Research and Public Health 12(4): 4214-4230; Dai, X., Luo. F., Zhang. D., Dai, L., Chen, Y., and Dong. B. 2015. “Waste-activated Sludge Fermentation for Polyacrylamide Biodegradation Improved by Anaerobic Hydrolysis and Key Microorganisms Involved in Biological Polyacrylamide Removal,” (Nature Research) Scientific Reports 5: 11675; Wang, Z., Li, S., Wang, L., Zhang, S., Jiang, Y., Chen, J., and Luo, D. 2012. “Polyacrylamide Hydrogel Injection for Breast Augmentation: Another Injectable Failure,” Medical Science Monitor 18(6): CR399-CR408).

Wetting the urothelium distal to the jacket would then necessitate continuation of the tubular portion of the accessory channel into the distal lumen. To avoid this, the accessory channel is continued into an open-bottom groove that runs centrally along the bottom of the diversion chute to its distal tip. Second, in any embodiment, were the sides of the chute less than fully shape compliant with the changing diameter of the ureter as the chute is advanced, retracted, or left in the closed position, and the diameter changing due to the longitudinal peristalsis characteristic of the ureters, the press of the chute edges against the lumen wall would likely result in abrasion and incisions.

The chute, with slightly turned up edges to completely obturate the ureteral lumen and prevent leaks, is slightly larger than the largest internal diameter of the ureter during peristalsis, thus closing off completely the leakage of urine to either of its sides. Authoritative information on the range of normal dimensions of the adult human ureter is sparse. The largest mean resting external diameter of the living normal adult human ureter on unenhanced computed tomography is 2.7 to 3.0 millimeters (Zelenko, N., Coll. D., Rosenfeld, A. T., and Smith, R. C. 2004, Op cit.). Documentation in the literature on the width of the normal adult ureteral wall is likewise scarce, but may be taken as having a mean thickness of 0.9 to 1.0 millimeters.

For the present purpose, deviation from this value due to gender or body size is not significant, the soft rubbery or spongy highly elastic character of the diversion chute and compliance of the ureteral wall making a chute width of 2.0 millimeters applicable in 2 sigma, or 95 percent, of normal adult ureteral lumina when passing a urine bolus. For the present purpose, the diameter of the normal adult ureteral wall when quiescent may be taken as 0.9 to 1.0 millimeters. If significantly deviant from these values, meaning that the elasticity of the chute will not adjust during peristalsis to completely seal off the ureteral lumen, the trepan end-piece and diversion chute are custom made. For a small child, these values for stocked ureteral takeoff jackets are generally halved.

The compliant elasticity and bending bias molded into the diversion chute assure that incisions, gouges, and breaches for urine to continue past the chute will not occur. To eliminate the need for precise adjustment midprocedurally and dynamically adapt to individual variance in the internal diameter of the ureter during the passage of stones, the chute, as indicated, is made of a soft nonallergenic rubbery or spongy polymer such as hardness-adjusted expanded polytetrafluoroethylene (see, for example, Liu, X., Zhang, Y., Wang, S., Lei, Z., Li, X., and Fan, D. 2016. “The Use of Expanded Polytetrafluoroethylene in Depressed Deformities of the Face,” Experimental and Therapeutic Medicine 12(5): 3151-3154; Redbord, K. P. and Hanke, C. W. 2008. “Expanded Polytetrafluoroethylene Implants for Soft-tissue Augmentation: 5-year follow-up and literature review,” Dermatologic Surgery 34(6): 735-743) or prepared from a four percent polyalkylimide suspension in water trademarked Bioalchamid (see, for example, Honig, J. 2008. “Cheek Augmentation with Bio-Alcamid in Facial Lipoatrophy in HIV Seropositive Patients,” Journal of Craniofacial Surgery 19(4): 1085-1088; Lahiri. A. and Waters. R. 2007. “Experience with Bio-Alcamid, a New Soft Tissue Endoprosthesis,” Journal of Plastic, Reconstructive, and Aesthetic Surgery 60(6): 663-667; Pacini, S., Ruggiero, M., Morucci, G., Cammarota, N., Protopapa, C., and Gulisano, M. 2002. “Bio-alcamid: A Novelty for Reconstructive and Cosmetic Surgery,” Italian Journal of Anatomy and Embryology 107(3): 209-214) 2.0 millimeters wide with upwardly molded soft rounded edges.

Injected while fluid in larger volume just beneath the integument as in facial surgery, rather than in the preformed semisolid state pertinent to the present context, fill material is more susceptible to contamination (Nadarajah, J. T., Collins, M., Raboud, J., Su. D., Rao, K., Loutfy, M. R., and Walmsley, S. 2012. “Infectious Complications of Bio-Alcamid Filler Used for HIV-related Facial Lipoatrophy,” Clinical Infectious Diseases 55(11): 1568-1574; Karim, R. B., Hage, J. J., van Rozelaar, L., Lange, C. A., and Raaijmakers, J. 2006. “Complications of Polyalkylimide 4% Injections (Bio-Alcamid): A Report of 18 Cases,” Journal of Plastic, Reconstructive, and Aesthetic Surgery 59(12): 1409-1414), and instances of migration following injection (Jordan, D. R. and Stoica, B. 2015. “Filler Migration: A Number of Mechanisms to Consider,” Ophthalmic Plastic and Reconstructive Surgery 31(4): 257-262; Khan, I., Shokrollahi, K., Bisarya, K., and Murison, M. S. 2011. “A Liposuction Technique for Extraction of Bio-Alcamid and Other Permanent Fillers,” Aesthetic Surgery Journal 31(3): 344-346) have no more relevance to use within a ureter than does the leukocytopenia reported following the injection of polyacrylamide (Rong. L., Lan, S., Shao, Y., Che, Z., and Zhang, D. 2015. “A Case of Special Complication following a Large Amount of Polyacrylamide Hydrogel Injected into the Epicranial Aponeurosis: Leukocytopenia,” Case Reports in Medicine 2015:695359).

When pressed against the transitional cell epithelium lining, or urothelium, the edges of the chute continue to bend upwards rather than incise or gouge the urothelial lining. The flexibility of the chute allows the leading edge to extend 2.0 millimeters forward of the trepan to fully obturate almost any normal human adult ureter. In both adjustable and nonadjustable embodiments, compression, incisions, and gouges of the urothelium by the outer edges of the chute are avoided through the use of a soft and elastic suitably molded chute that dynamically changes shape to comply with the instantaneous internal diameter of the ureter.

In an adjustable embodiment, it is necessary to prevent the complete covering over (overlayment, masking, blanketing) and compression of the urothelium by the outlet obturator. Compression is avoided due to the elasticity of the chute to which the outlet obturator is fastened. The complete covering over of the urothelium by the outlet obturator is accomplished by liberally perforating the obturator with 0.13-millimeter holes, which are too small to allow urine boluses to pass through and enter the outlet when the obturator is retracted to allow urine to continue to the lower tract. Any seepage in this regard would simply pass to the collection bag. Minimizing compression not only protects the urothelium and suburothelium from injury but allows relatively normal neurophysiological function of the urinary tract.

Extended constriction of the urothelium and suburothelium, or the mucosa lining the urinary tract except during voiding can lead to physical injury that would facilitate infection, but no less significantly, can interfere with the function of these tissue layers, which more recent evidence has established do not comprise a ‘dumb’ conduit lining to fend off infection but are communicative, or ‘smart,’ While patients with a prosthesis will be missing part if not all of the lower tract, those wearing an adjustable embodiment will retain partially, temporarily impaired, or completely normal structure and function, which should be disturbed as little as possible.

Much as the vascular endothelium by its neuromuscular and secretory function exerts far-reaching as well as local effects upon circulation, the tissue lining the urinary tract releases adenosine, adenosine triphosphate, nitric oxide, and acetylcholine to modulate its underlying smooth muscle as an essential factor in the initiation of voiding, for example (see, for example, Merrill. L., Gonzalez, E. J., Girard, B. M., and Vizzard, M. A. 2016. “Receptors, Channels, and Signaling in the Urothelial Sensory System in the Bladder,” Nature Reviews. Urology 13(4): 193-204; d'Emmanuele di Villa Bianca, R., Mitidieri. E., Fusco, F., Russo, A., Pagliara, V., Tramontan, T., Donnarumma, E., and 4 others 2016. “Urothelium Muscarinic Activation Phosphorylates CBS(Ser227) via cGMP/PKG Pathway Causing Human Bladder Relaxation through H₂S Production,” (Nature Research) Scientific Reports 6:31491; d'Emmanuele di Villa Bianca. R., Mitidieri, E., Esposito, D., Donnarumma, E2., Russo, A., Fusco, F., lanaro, A., and 4 others 2015. “Human Cystathionine-β-Synthase Phosphorylation on Serine227 Modulates Hydrogen Sulfide Production in Human Urothelium” Online, PLoS [Public Library of Science] One 10(9): e0136859; Winder, M., Tobin, G., Zupančić. D., and Romih, R. 2014. “Signalling Molecules in The Urothelium,” Online, Biomed Research International 2014:297295; Birder, L. A. and Andersson, K. E. 2013. “Urothelial Signaling,” Physiological Reviews 93(2): 653-680; Birder, L. A. 2013. “Nervous Network for Lower Urinary Tract Function,” International Journal of Urology 20(1): 4-12; Birder, L. A., Kanai, A. J., Cruz, F., Moore, K., and Fry, C. H. 2010. “Is the Urothelium Intelligent?,” Neurourology and Urodynamics 29(4): 598-602; Schnegelsberg, B., Sun, T. T., Cain, G., Bhattacharya, A., Nunn, P. A., Ford, A. P., Vizzard, M. A., and Cockayne, D. A. 2010. “Overexpression of NGF [nerve growth factor] in Mouse Urothelium Leads to Neuronal Hyperinnervation, Pelvic Sensitivity, and Changes in Urinary Bladder Function,” American Journal of Physiology. Regulatory, Integrative, and Comparative Physiology 298(3): R534-R547).

This dynamic adaptive function is essential for normal adjustment to the ordinary internal and environmental as well as pathophysiological stresses that affect urinary function. Futhermore, interference with urothelial neural and/or chemical signaling function can be the cause of existing or can induce new urinary dysfunction (see, for example, Kanai, A., Fry, C., Ikeda, Y., Kullmann, F. A., Parsons, B., and Birder, L. A. 2016. “Implications for Bidirectional Signaling between Afferent Nerves and Urothelial Cells-ICI-RS [International Consultation on Incontinence-research Society meeting, Bristol, England] 2014,” Neurourology and Urodynamics 35(2): 273-277; Keay, S. K., Birder, L. A., and Chai, T. C. 2014. “Evidence for Bladder Urothelial Pathophysiology in Functional Bladder Disorders,” Online, Biomed Research International 2014:865463; Yoshimura, N., Kaiho, Y., Miyazato, M., Yunoki, T., Tai, C., Chancellor, M. B., and Tyagi, P. 2008. “Therapeutic Receptor Targets for Lower Urinary Tract Dysfunction,” Naunyn Schmiedebergs Archives of Pharmacology 377(4-6): 437-448; Jung, S. Y., Fraser, M. O., Ozawa, H., Yokoyama, O., Yoshiyama, M., De Groat, W. C., and Chancellor, M. B. 1999. “Urethral Afferent Nerve Activity Affects the Micturition Reflex; Implication for the Relationship between Stress Incontinence and Detrusor Instability,” Journal of Urology (Philadelphia, Pennsylvania) 162(1): 204-212).

Normal urinary function demands intact bidirectional communication between central and peripheral systems, to include the urothelium lining the urethra and the tissue of the urethral sphincters (see, for example Yoshimura, N., Ogawa, T., Miyazato, M., Kitta, T., Furuta, A., Chancellor, M. B., and Tyagi, P. 2014. “Neural Mechanisms Underlying Lower Urinary Tract Dysfunction,” Korean Journal of Urology 55(2): 81-90; Rocha, J. N. 2016. “Effect of S-methyl-1-thiocitrulline Dihydrochloride on Rat Micturition Reflex,” International Brazilian Journal of Urology 42(5): 1018-1027). Outlet (internal urethral orifice, uvula vesicae, and neck) obstruction having been addressed in U.S. Pat. No. 11,013,858, this application pertains primarily to urinary incontinence.

Requiring at least one hole to be open to the outside for urine to drain, the surface port used for urinary diversion differs from surface ports used only to inject drugs in that the drain hole opens to the outside and is not covered over by the skin. Barring the presence of an interposed drainage reservoir or neobladder, the urine outlet hole leads directly into the jacket mainline. In contrast, when only drug openings are present, the surface port is fully implanted subdermally or subcutaneously, that is, just inside the integument, with drug delivery into the sidelines or accessory channels by jet injection. When drug administration is automated to effect release at programed intervals, the drug openings lead into reservoirs interposed between the entry openings and the lines leading to the target site respective of each hole-reservoir-line channel.

When not gravity fed upon the opening of a valve, withdrawal of each dose is by a pump at the outlet to each reservoir, the pump actuated by a timing controller or microcontroller, these drug delivery components all small and fully implanted. When the need to be able to pass a cabled device or devices through one or more drug delivery openings in the surface port on a frequent basis is established before the port is placed, the junction of the sideline (accessory channel, service channel) respective a frequent passage through a hole in the surface port other than the drain is angled to facilitate passing of the device into the mainline and native ductus.

An exceptional need to pass a cabled device through a sideline is accomplished by slitting the skin overlying the opening and thereafter sealing the slit with surgical cyanoacrylate cement. The direct targeting of drugs in this manner allows antimicrobials, for example, to be delivered in greater concentration to eradicate a bladder, or lower urinary tract infection, and an anesthetic to alleviate the discomfort. Another difference is that urinary diversion is an excurrent or outflow function, without an incurrent drug delivery metering or dose dispensing size and timing regimen. Instead, operation is continuous, urine passively diverted as it is produced.

In a drug infusion application, the implant set will usually include one or more small flat drug storage reservoirs typically positioned subdermally in the pectoral region. An excurrent function lacking, the port will not include an opening to the outside, instead being overlain with skin, hence, fully implanted, through which reservoir replenishment is by jet injection. The timed delivery of one or more drugs is controlled by an implant timing module or microcontroller which actuates small pumps connected to the reservoirs. If appropriate, the timing of drug or maintenance agent delivery can be triggered by the pressure actuated switch in the neoureter convergence chamber.

Much leeway in internal diameter is afforded by the foam lining the jacket, which can be made thicker to allow for growth or swelling. Only in more extreme cases, as a bladder exstrophic neonate, should it eventually become necessary to explant and replace the jacket. Takeoff at the ureters precedes the bladder, and therewith, bladder dysfunction or disease regardless of type, and preempts all sense of urgency, instead diverting urine passively and unconsciously to a paracorporeal collection bag. The takeoff jacket to either side can be positioned at any level along its respective ureter, and each jacket communicates by means of an accessory channel (service channel, sideline) with an entry portal at the body surface through which the urine diversion line exits and the accessory channels enter.

Thus, implant-maintaining substances can be targeted directly to the jackets and synthetic lines and drugs to any preserved length of the ureter. Takeoff at any level along either ureter keeps urine from contact with, while allowing the direct delivery of medication to, the lower tract. This isolation of the lower tract facilitates healing following surgery of the lower tract and/or the application of medication to treat a disease condition where systemic medication would otherwise have to be given at higher doses with nontargeted tissue exposed and washing over by urine of topically applied medication would flush away and dilute medication to impede recovery and necessitate more frequent dosing at increased expense. This is true when the urine is nonpathogenic in health, much less when it is infectious in disease.

Accessory or service channels of nonjacketing side-entry connectors inserted into the parenchyma of a solid organ tumor can be radiation shielded to allow treatment with lower dose rate radionuclides (radioisotopes). In contrast to a blood vessel, which is continuous in flow-through so that the lumen remains patent, the lumen of a ureter remains substantially unchanged in outer diameter while the tissues lining the lumen compress during urine outflow to increase the luminal cross-sectional area some 27-fold (Woodburne, R. T and Lapides, J. 1972. “The Ureteral Lumen during Peristalsis,” American Journal of Anatomy 133(3): 255-258). When the surgical procedure is unilateral, only the one side need be addressed thus.

While in this case installation is temporary, the apparatus is best removed following healing; however, removal, especially if accomplished robotically, is by entry through a small “keyhole,” incision. Unlike the clearly patent lumen of a blood vessel, to prevent the trepan from cutting into the opposing wall, the tiny lumen of a ureter requires to be protected by the temporary insertion of a stent or lightly insufflated. Direct connection of the ureters to the skin without involving the gut, or ureterocutaneostomy, was soon realized to result in complications, primarily ureteral stenosis.

Now it is practiced only as an exigent measure when the gut is unusable or the patient has little lifetime remaining (see, for example, Stein, R., Hohenfellner, M., Pahernik, S., Roth, S., Thüroff. J. W., and Rubben, H. 2012. Op cit. above in this section). The construction of an ileal conduit, the first means of urinary diversion realized using various techniques developed in the 1940s by Eugene M. Bricker and eponymously designated a Bricker conduit, begins with the harvesting of a 15 centimeter segment of ileum at a distance of 15 centimeters from the ileocecal valve.

An Indiana pouch, developed by a team at the Indiana University Medical Center, provides a continent internal reservoir or neobladder, as do several alternative procedures. Construction begins with the harvesting of a length of ileum plus 2 feet of the ascending colon. Usually successful in experienced hands, these initial procedures in creating a ureteroenteric anastomosis nevertheless impair the gut and risk complications even before the ileal segment taken is tubularized, or tabulated, and resituated.

In what is essentially a second procedure, the ileal and colonic tissue is diverted to a different function for which its lining is not adapted, previously irradiated tissue can undergo postprocedural degeneration and the development of abscesses, to which synthetics are oblivious, and postprocedural stenting and its complications are averted (see, for example, Rosenzweig, B., Mor. Y., Erlich, T., Laufer, M., Winkler, H., Kaver, I., Ramon, J., and Dotan, Z. A. 2016, Op cit.; Takeuchi, M., Masumori, N., and Tsukamoto, T. 2014. “Ureteral Reconstruction with Bowel Segments: Experience with Eight Patients in a Single Institute,” Korean Journal of Urology 55(11): 742-749; Krajewski, W., Piszczek, R., Krajewska, M., Dembowski, J., and Zdrojowy, R. 2014. “Urinary Diversion Metabolic Complications-Underestimated Problem,” Advances in Clinical and Experimental Medicine 23(4): 633-638: “ . . . the absorptive-secretory function of used bowel segment is intact . . . ”, Stein, R., Hohenfellner, M., Pahemik, S., Roth, S., Thuroff, J. W., and Rubben, H. 2012. Op cit.:” . . . the resorptive surface of the bowel segment used can no longer play its original physiological role in the gastrointestinal tract, even though its absorptive and secretory functions are still intact. . . . This has metabolic consequences, because the diverted urine here comes into contact with a large area of bowel epithelium. Early preventive treatment must be provided against potentially serious complications such as metabolic acidosis and loss of bone density.

The resection of ileal segments can also lead to malabsorption. The risk of secondary malignancy is elevated after either continence-preserving anal urinary diversion (>2%) or bladder augmentation (>1%)”; Lazica, D. A., Ubrig. B., Brandt, A. S., von Rundstedt, F. C., and Roth, S. 2012. “Ureteral Substitution with Reconfigured Colon: Long-term Follow-up,” Journal of Urology (Philadelphia, Pennsylvania) 187(2): 542-548. The same applies to any procedure that channels urine through gut, whether an ileal conduit, continent ureteroileocystoplasty, or a neobladder constructed from gut, the former posing the risks associated with a stoma, the latter two more susceptible to midprocedural complications, and all subject to the degradation of digestive tissue with constant exposure to urine. Another problem with some stomata is that digestive enzymes and acids which come into contact with the skin cause much irritation.

Following the insertion into the urinary tract, the transition of gut to malignancy apparently takes about 15 years, making such use unsound in patients less than age 60 and the correct application of synthetics the only route to avoid major complications for the foreseeable future (see, for example Rosenzweig, B., Mor, Y., Erlich, T., Laufer, M., Winkler, H., Kaver, I., Ramon, J., and Dotan, Z. A. 2016, Op cit.: Wiestma, A. C., Estrada, C. R. Jr., Cho, P. S., Hollis, M. V., and Yu, R. N. 2016. “Robotic-assisted Laparoscopic Bladder Augmentation in the Pediatric Patient,” Journal of Pediatric Urology 12(5): 313.e1-313.e2; Stewart, D., Inouye, B. M., Goldstein, S. D., Shah, B. B., Massanyi, E. Z., and 5 others 2015. “Pediatric Surgical Complications of Major Genitourinary Reconstruction in the Exstrophy-epispadias Complex,” Journal of Pediatric Surgery 50(1): 167-170; Merriman, L. S., Arlen, A. M., Kirch, A. J., Leong. T., and Smith, E. A. 2015. “Does Augmentation Cystoplasty with Continent Reconstruction at a Young Age Increase the Risk of Complications or Secondary Surgeries?,” Journal of Pediatric Urology 11(1): 41.e1-e5; Inouye, B. M., Shah, B. B., Massanyi, E. Z., Di Carlo, H. N., Kem, A. J., and 4 others 2014. “Urologic Complications of Major Genitourinary Reconstruction in the Exstrophy-epispadias Complex,” Journal of Pediatric Urology 10(4): 680-687; Ordorica, R., Wiegand, L. R., Webster, J. C., and Lockhart, J. L. 2014. “Ureteral Replacement and Onlay Repair with Reconfigured Intestinal Segments,” Journal of Urology (Philadelphia, Pennsylvania) 191(5): 1301-1306; Chowdhary, S. K., Rao, K. L., Kandpal, D. K., Sibal. A., and Srivastava, R. N. 2014. “Indiana Pouch in Children: A 15-year Experience,” Journal of Pediatric Urology 10(5): 911-916).

Pending the successful use of engineered tissue urothelium lined conduits, the use of synthetic materials, especially in the young, will remain the best option for averting the inception of malignancy; however, to generate tissue from stem cells taken from the patient may require more time than the condition of the patient may allow, and the technology remains far from practicable, making the use of synthetics advantageous for now (see, for example, Alberti, C. 2016. “Whyever Bladder Tissue Engineering Clinical Applications Still Remain Unusual Even Though Many Intriguing Technological Advances Have been Reached?,” Giornali de Chirurgia [journal of surgery] 37(1): 6-12; Smolar, J., Salemi, S., Horst. M., Sulser. T., and Eberli. D. 2016. “Stem Cells in Functional Bladder Engineering,” Transfusion Medicine and Hemotherapy 43(5): 328-335; Zhao, Z., Yu, H., Fan, C., Kong, Q., Liu, D., and Meng. L. 2016. “Differentiate into Urothelium and Smooth Muscle Cells from Adipose Tissue-derived Stem Cells for Ureter Reconstruction in a Rabbit Model,” American Journal of Translational Research 8(9): 3757-3768; Stewart, D., Inouye, B. M., Goldstein, S. D., Shah, B. B., Massanyi. E. Z., and 5 others 2015, Op. cit.; Xu. Y., Fu. W., Wang, Z., Li, G., and Zhang, X. 2015. “A Tissue-specific Scaffold for Tissue Engineering-based Ureteral Reconstruction,” Online, PLoS [Public Library of Science] One 10(3): e0120244; Zhu, W. D., Xu, Y. M., Feng. C., Fu, Q., Song. L. J., and Cui. L. 2010. “Bladder Reconstruction with Adipose-derived Stem Cell-seeded Bladder Acellular Matrix Grafts Improve Morphology Composition,” World Journal of Urology 28(4): 493-498).

Conventional procedures which pass urine through urothelium, such as a ureterocutaneostomy are to be preferred, although the stoma will still pose complications. The intestinal mucosa is damaged by constant exposure to urine, making the transplant prone to metaplastic transition, which—with greater likelihood the younger is the patient—can ultimately progress, apparently de novo, first to primary premalignancy in the form of benign adenoma (see, for example, Manka, M. G., Santoni, C., Wright, E. J., and Gearhart, S. L. 2015. “Tubular Adenoma in the Indiana Pouch of a Patient with a History of Bladder Exstrophy,” Urology Case Reports 3(5): 141-142), and thence to frank malignancy in the form of adenocarcinoma (see, for example, Weyant, G. W., Karamchandani, D. M. 1, and Rassaei, N. 2015. “Colorectal Microcarcinoids in Association with Long-term Exposure to Urinary Content: A Case Report and Review of the Literature” Case Reports in Pathology 2015:806310; Matulewicz, R. S., Fryer, J. P., Yang, X. J., Goyal. R., and Hairston, J. C. 2015. “Renal Transplantation in the Setting of Prior Urinary Diversion: A Case of Poorly Differentiated Adenocarcinoma in an Ileal Conduit,” Urology Case Reports 3(3): 53-55; Vasdev. N., Moon, A., and Thorpe, A. C. 2013. “Metabolic Complications of Urinary Intestinal Diversion,” Indian Journal of Urology 29(4): 310-315; Jian, P. Y., Godoy, G., Cobum, M., Lynch, G., Ro. J. Y., Zhai, Q. J., Nishino, M., and Lerner, S. P. 2012. “Adenocarcinoma Following Urinary Diversion,” Canadian Urological Association Journal 6(2): E77-E80; e1-Khalil, S. A. 2010. “Long-term Complications following Ileal Conduit Urinary Diversion after Radical Cystectomy,” Journal of the Egyptian National Cancer Institute 22(1): 13-18; Ali-El-Dein, B., El-Tabey, N., Abdel-Latif, M., Abdel-Rahim, M., and El-Bahnasawy, M. S. 2002. “Late Uro-ileal Cancer after Incorporation of Ileum into the Urinary Tract,” Journal of Urology (Philadelphia, Pennsylvania) 167(1): 84-88; Tsuboniwa, N., Miki, T., Kuroda, M., Maeda, O., Saiki, S., Kinouchi, T., Usami, M., and Kotake. T. 1996. “Primary Adenocarcinoma in an Ileal Conduit,” International Journal of Urology 3(1): 64-66; Sakano, S., Yoshihiro, S., Joko, K., Kawano, H., and Naito, K. 1995. “Adenocarcinoma Developing in an Ileal Conduit” Journal of Urology (Philadelphia, Pennsylvania) 153(1): 146-148; Shokeir, A. A., Shamaa. M., e1-Mekresh, M. M., e1-Baz, M., and Ghoneim, M. A. 1995. “Late Malignancy in Bowel Segments Exposed to Urine Without Fecal Stream,” Urology 46(5): 657-661; Thomas, D. J., Goble, N. M., Gillatt, D. A., Hammonds, J. C., and Smith, P. J. 1990. “Histological and Environmental Changes in Longstanding Ileal Conduits,” Journal of the Royal Society of Medicine 83(9): 557-558). In otherwise substantially healthy young children, this metaplastic transition appears to take longer than 15 years (Wiestma, A. C., Estrada, C. R. Jr., Cho, P. S., Hollis, M. V., and Yu, R. N. 2016, Op. cit.; Chowdhary, S. K., Rao, K. L., Kandpal, D. K., Sibal, A., and Srivastava, R. N. 2014, Op. cit), making an initial reassignment of gut a viable means for bridging the gap until the incongruous tissue can be removed, preferably, to be replaced with synthetic materials.

Furthermore, it has been known for almost four decades that apart from de novo, or primary, development of carcinoma in the conduit due to histological and physiological incompatibility with urine, a radical cystectomy with the creation of an ileal conduit responsive to preexisting bladder malignancy can eventuate in secondary malignancy of the conduit as well as the upper urinary tract (Kalble, T., Hofmann, I., Thuroff, J. W., Stein, R., Hautmann, R., Riedmiller, H., Vergho, D., and 47 others 2012. “Secondary Malignancies in Urinary Diversions,” (in German with abstract at Pubmed), Urologe. Ausgabe A 51(4): 500-506; Austen, M. and Kalble, T. 2004. “Secondary Malignancies in Different Forms of Urinary Diversion Using Isolated Gut,” Journal of Urology (Philadelphia, Pennsylvania) 172(3): 831-838; Shokeir, A. A., Shamaa. M., e1-Mekresh, M. M., e1-Baz, M. and Ghoneim, M. A. 1995. “Later Malignancy in Bowel Segments Exposed to Urine without Fecal Stream,” Urology 46(5): 657-661; Curran, F. T and Fuggle, W. J. 1986. “Transitional Cell Carcinoma in an Ileal Conduit,” Postgraduate Medical Journal 62(730): 769-771; Moskovitz, B. and Levin, D. R. 1986. “Recurrent Transitional Cell Carcinoma in an Ileal Conduit,” Urologia Internationalis 41(3): 225-227; Rubin, B. E., Rodriguez, E., Mangasarian, R., Cummings, J., and Kwart, A. 1979. “Recurrent Transitional Cell Carcinoma in an Ileal Conduit,” Urologie Radiology 1(1): 61-62).

Repositioned gut is susceptible to the development of adenocarcinoma even when not exposed to the erosive effect of urine (see, for example, Sterpetti, A. V., Marzo, L. D., and Sapienza, P. 2021. “Risk Factors for Adenocarcinoma in the Surgically Transposed Colon Not Exposed to the Fecal Stream. Etiological Considerations Extrapolated to Sporadic Colon Carcinoma in the General Population,” European Journal of Surgical Oncology 47(5): 931-934; Sterpetti, A. V., Costi, U., Grande, R., D'Ermo, G. and Sapienza, P. 2020. “De Novo Secondary Adenocarcinoma in the Colon Used as Urinary Diversion Not in Contact with the Fecal Stream: Systematic Review and Meta-analysis,” Annals of Surgical Oncology 27(8): 2750-2759; Simoes, C., Moura, M., Noronha Ferreira, C., Rosa. R., Freire, J. P., Carillho, Ribeiro, L., and Tato Marinho, R. 2019. “Lateral Spreading Tumor Arising in an Interposed Colonic Segment,” Online, ACG [American College of Gastroenterology] Case Reports Journal 6(10): e00245; Sterpetti, A. V. and Sapienza, P. 2019. “Adenocarcinoma in the Transposed Colon: High Grade Active Inflammation versus Low Grade Chronic Inflammation,” European Journal of Surgical Oncology 45(9): 1536-1541; Barbosa, B., Santos, B., Mesquita, I., Marcos, M., Nogueira, C., and Santos, J. 2018. “Adenocarcinoma Arising in a Colonic Interposition after Esophagectomy for Benign Stricture and Review of the Literature,” Online, Journal of Surgical Case Reports 2018(10): riy264; De Moura, D. T. H., Ribeiro, I. B., Coronel M., De Moura, E. T. H., Carvalho, J. R., Baba, E. R., and De Moura, E. G. H. 2018. “Primary Adenocarcinoma Arising in Esophageal Colon Interposition for Corrosive Esophageal Injury: A Case Report and Review of the Literature,” Online, Endoscopy International Open 6(12): E1406-E1409; Taslimi, R., Jowkar, A. Hasani Ghavam, M. R., Tavasol, T., Allameh, S. F., and Rakhshani, N., 2017. “Squamous Cell Carcinoma (SCC) Arising in Esophageal Colon Interposition,” Online, Oxford Medical Case Reports 2017(12): omx068; Ng. D. W., Ching Tan, G. H., and Teo. M. C. 2016. “Malignancy Arising in a 41-year-old Colonic Interposition Graft,” Online, Asian Journal of Surgery 39(1): 45-47; Aryal, M. R., Mainali, N. R., Jalota, L., and Altomare, J. F. 2013. “Advanced Adenocarcinoma in a Colonic Interposition Segment,” Online, British Medical Journal Case Reports 2013: ber2013009749; Fritscher-Ravens, A., Sriran, P. V., Thonke, F., Jaeckle, S., Maydeo, A. and Soehendra, N. 1999. “Synchronous Adenocarcinoma in the Transposed Colonic Conduit after Esophagectomy for Squamous Cell Cancer: Endoscopic Palliative Resection while Awaiting Surgery,” Gastrointestinal Endoscopy 50(6): 852-854).

Such consequences are possible whether the diversion fabricated from gut is a pouch according to the Bricker, Indiana, Wallace, Wallace I I, Mainz I I, Koch, Florida, Miami. Tpouch, Bricker end-to-side, Le Duc, or any of several variants thereof (see, for example, McKieman, J. M., DeCastro, G. J., and Benson, M. C. 2015. “Cutaneous Continent Urinary Diversion,” in Wein, A. J., Kavoussi, L. R., Partin, A. W. and Peters C. A. (eds.), Campell-Walsh Urology, 11th ed., Part XIII, Benign and Malignant Bladder Disorders, Chapter 98, New York, New York: Elsevier; Krajewski. W., Piszczek, R., Krajewska, M., Dembowski, J., and Zdrojowy, R. 2014, Op cit; Obenauer, S., Plothe, K. D., Ringert. R. H., Grabbe, E., and Heuser, M. 2007. “Forms of Urinary Diversion-Methods and Imaging Findings,” (in German with English abstract at Pubmed), Rofo [Röntgenstrahlen Fortschritte (Advances in Radiology) 179(10): 1025-1034; Ashken, M. H. (ed.) 1982. Clinical Practice in Urology: Urinary Diversion, New York, New York: Springer).

While urinary rather than fecal diversion is addressed, numerous complications such as leaks, infection, peristomal irritation and dermatitis, retraction, prolapse, stenosis, hernia, necrosis, and ileocutaneous fistula, pertain to both urinary and fecal stomas, or stomata. In a cancer patient, stoma resolution or the need for corrective measures can delay chemotherapy (Sherman, K. L. and Wexner, S. D. 2017. “Considerations in Stoma Reversal,” Online, Clinics in Colon and Rectal Surgery (New York, New York) 30(3): 172-177). Even though the risks of malignancy and stoma complications rise with the length of time the tissue taken from the gut remains in use, many such procedures are performed, to include on young patients.

Where such need arises, the use of synthetic materials eliminates the need for a preliminary operation with its own potential complications. U.S. Pat. No. 11,389,171, entitled Integrated System for the Infixion and Retrieval of Implants, addresses the use of local drug delivery jackets to skip lesions in Crohn's disease. These earlier drug delivery jackets were replenished through the systemic infusion of medication which was magnetically extracted by each jacket-later jackets have medication directly piped to them upon signaling by sensors local to the lesions by the system controller of the need therefor. The former method eliminates the need to route a druglines subcutaneously to each jacket.

A preliminary operation to harvest tissue from the ileum is best avoided (see, for example, Doğru, V., Akova, U., Esen, E., Wong, D. J., da Luz Moreira, A, and 4 others 2024. “Temporary Diverting Loop Ileostomy in Crohn's Disease Surgery Indications and Outcome,” Langenbeck's Archives of Surgery (Berlin, Germany) 409(1): 247; Sam, S. W., Hafeez, B., Ong. H. I., Gill, S., Smibert, O., and 4 others 2024. “The Impact of Faecal Diversion on the Gut Microbiome: A Systematic Review,” Online, Gut Microbiome (Cambridge, England) 5: e4; Plasencia, A. and Bahna, H. 2019. “Diverting Ostomy: For Whom, When, What, Where, and Why,” Online, Clinics in Colon and Rectal Surgery (New York, New York) 32(3): 171-175; Lightner, A. L. and Pemberton, J. H. 2017. “The Role of Temporary Fecal Diversion,” Online, Clinics in Colon and Rectal Surgery (New York, New York) 30(3): 178-183).

Moreover, unlike the use of prosthetic means, surgical diversion is far more likely to necessitate a second, reconstruction procedure to correct the original operation, and even when temporary, reversal demands a second surgical procedure which risks more complications to reinstate normal structure and function than does the use of synthetic parts (see, for example, Roig, J. V., Salvador, A., Frasson, M., Garcia-Mayor. L., Espinosa, J., and 8 others 2018. “Stora Reversal after Surgery for Complicated Acute Diverticulitis: A Multicentre Retrospective Study,” (in Spanish and English) Cirugia Española (Barcelona, Spain) 96(5): 283-291; den Dulk, M., Smit, M., Peeters, K. C., Kranenbarg, E. M., Rutten, H. J., and 3 others 2007. “A Multivariate Analysis of Limiting Factors for Storna Reversal in Patients with Rectal Cancer Entered into the Total Mesorectal Excision (TME) Trial: A Retrospective Study,” Lancet. Oncology (London, England) 8(4): 297-303).

Irreversible without risking numerous complications, an ileal conduit should not be considered for the distinct majority of patients, who present with a number of different bladder disorders which may be temporary, better treated were the bladder bypassed, or respond, if incompletely or slowly, to drug or electrostimulatory therapy, as to leave a consequential surgical procedure without justification. The advent of nonproteinaceous, nonallergenic tubing was immediately recognized as offering the benefit of not having to harvest autologous tissue with its attendant risks.

It soon emerged, however, that placed in the urinary tract, the accretion of crystal on the inner wall posed a risk, that placed in the arterial tree, there would quickly become an accumulation of thrombus along the internal surface, and that situated anywhere in the body, infectious biofilm can accumulate as a potential threat to patency as well as health. Occlusion and contamination ended the practice of using small caliber tubing as a substitute for the harvesting of a native ductus, such as the great saphenous vein and later, the internal thoracic artery, which also resulted in complications at the harvest sites. Today synthetic tubing is used only to bypass the largest vessels as most elusive of reconstruction from autologous tissue and least susceptible to occlusion.

Synthetic materials are not susceptible to complications such as stricture, infection, metaplastic transition, shear stress, or stomal deterioration, and the incorporation of accessory or service channels into the ureteral takeoff jackets allows biofilm, coagulation, and accretion-preventive agents to be drip released into the lines as necessary. Moreover, adverse tissue reactions or other complications associated with the implantation of synthetics are readily overcome. Unlike a conduit and stoma constructed from native tissue, the parts in a synthetic assembly fit together precisely, precluding leakage and are easily fitted with sensors as part of an automatic prosthetic disorder response system, which depending upon the type and severity of the disorder, can be ambulatory.

Moreover, in surmounting the central problems of urine leakage and incorporating a drugline in an accessory channel to allow the directly pipe-targeting to the jacket and line of occlusion counteractants, a ductus side-entry jacket eliminates the incapacities of small caliber catheters that have prevented the use of these for permanent implantation. Dependably fixed in position by suture, anchoring round needles, and tissue ingrowth, the surface port for the excurrent passage of urine and the incurrent flow of drugs is unlikely to become dislodged or allow any of the complications that result from inadequate adhesion such as parastomal herniation or prolapse.

While less susceptible to clogging, the placement of a ductus side-entry jacket proximal to the superior end-to-end anastomosis of such a synthetic graft allows the direct targeting to the anastomotic junction and prosthesis of antimicrobial and/or anticoagulative medication as necessary. This is no less the case with synthetic endoluminal grafts such as those used to contain an abdominal aortic aneurysm. Antimicrobial, anti-inflammatory, anticoagulative, anesthetic, stone formation preventive, and other supportive drugs to maintain patency and cleanliness are delivered directly to the jackets, lines and urinary tract distal to the level along its respective ureter of each jacket.

Another fundamental advantage in the use of synthetics is clear superiority for the fitting of sensing and actuation electronics, a central consideration where automation must be enlisted to treat comorbid conditions such as cardiorenal syndrome in a patient incapable of self-care. Diversion to isolate a kidney transplant from the possibility of reflux is addressed unilaterally with an ipsilateral jacket having a diversion chute, mainline, or urine diversion channel, and accessory channels or sidelines to give direct access for the delivery of drugs or the passage of a cabled device, such as an endoscope or laser. Itself a type of nonjacketing side-entry connector, the surface port is not susceptible to degradation as is a stoma, and proper hygiene makes irritation or infection of the surrounding tissue unlikely.

The caliber of the lines determined before placement, a fine fiberscope can be passed through any of the openings in the surface port to view, manipulate, swab, or focus a laser at any level along the line. To pass the scope or other cable like device past the diversion chute, an intermittent embodiment that allows the chute to be retracted must be used. When use is to be intermittent or elective, such as only during the night, or when diversion is to be suspended to allow to allow medication to pass through a post diversion segment of the ureter or lower urinary tract to be treated, retraction and advancement of the chute is with a push/pull control cable. Control thus averts the need for complex and costly electronics.

In FIG. 30, the upper accessory channel (sideline, drugline) catheters 8 and 8′ input into the valves and are separate from the Bowden push/pull control cables. Electrical wires as might be used to connect the master control microprocessor to one or more sensors positioned within the neobladder 145 to provide immediate urinalysis information in a multiply morbid patient, for example (see, for example, Toal. M., Fergie. R., Quinn, M., Hill. C., O'Neill, C., and Maxwell, A P. 2024. “Protocol for a Systematic Review of the Application of the Kidney Failure Risk Equation and Oxford Classification in Estimating Prognosis in IgA Nephropathy,” Online, [BioMed Central] Systematic Reviews (London, England) 13(1): 122; Terracina, S., Pallaria, A., Lucarelli, M., Angeloni, A., De Angelis. A., and 6 others 2023. “Urine Dipstick Analysis on Automated Platforms: Is a Reliable Screening Tool for Proteinuria? An Experience from Umberto I Hospital in Rome,” Online, MDPI [Multidisciplinary Digital Publishing Institute] Biomedicines (Basel, Switzerland) 11(4): 1174; Sumida, K., Nadkam,i G. N., Grams, M. E., Sang. Y., Ballew, S. H., and 30 others 2020. “Conversion of Urine Protein-Creatinine Ratio or Urine Dipstick Protein to Urine Albumin-Creatinine Ratio for Use in Chronic Kidney Disease Screening and Prognosis: An Individual Participant-based Meta-analysis,” Online, Annals of Internal Medicine (Philadelphia, Pennsylvania) 173(6): 426-435; Oyaert, M. and Delanghe, J. 2019. “Progress in Automated Urinalysis,” Online, Annals of Laboratory Medicine (Seoul, Korea) 39(1): 15-22), can be run in adjacent relation through double lumen catheters made of a slippery polymer or a fluoropolymer such as the polytetrafluoroethylene.

In FIG. 30, two pairs of druglines are provided, one toward the top of the FIGS. 8 and 8′ and another leading upward from the body surface port at the bottom of the figure also 8 and 8′. Entry through the latter into their respective valves is through a spring-loaded one-way cap (not shown) that prevents the leakage of urine in the caudal direction and allows the passing through of a prove to remove a test sample or inject drugs. Using the lower druglines, medication or a maintenance agent such as a crystal solvent can also be injected at the body surface port to pass up into the ureteral tap-off valves. The cable should be made with sufficient precision that a barrel adjuster at the surface port end for accessibility should be unnecessary, especially because adjustment if needed would be too small to achieve by hand.

Lower druglines 8 and 8′ run in parallel with the inner control wire push/pull control cables 28 and 28′, both from body surface port in FIG. 26C up to the valves where these can tap off urine samples for collection at a surface port such as that shown in FIG. 26C for microscopic and instrumental analysis able to provide data of greater specificity than implanted sensors alone (see, for example, Abusoglu, S., Guven, H., Ecer, B., Yorulmaz, A. E., Sivrikaya, A., and 4 others 2024. “Comparison of Labsan Tricell-1000 and Dirui FUS-2000 Automated Urine Analyzers with Manual Microscopy,” Online, Practical Laboratory Medicine (Amsterdam, North Holland, Netherlands) 39: e00386; Bai. L., Xu, Q., and Wu, Z. 2024. “Performance Analysis of Urine Formed Element Analyzer EH-2090 was Found to Have Good Accuracy in Detecting RBCs and WBCs When Compared to Manual Microscopic,” Online, Translational Andrology and Urology (Hong Kong, China) 13(2): 218-229; Shukuya, K., Morita, Y., Hisasue T., Ono, Y., Tomiyasu, S., Kurano. M., Yatomi, and Y., Tanaka, M. 2023. “Comparison of The Clinical Performance of the Atyp.C [atypical cells] Parameter of the [Sysmex] UF-5000 Fully Automated Urine Particle Analyzer with That of Microscopic Urine Sediment Analysis,” Online Practical Laboratory Medicine (Amsterdam, North Holland, Netherlands) 36: e00328; Yang, H., Smith, R. D., Sumner. K. P., Goodlett, D. R., Johnson, J. K., and Emst. R. K. 2022. “A Matrix-assisted Laser Desorption Ionization-Time of Flight Mass Spectrometry Direct-from-urine-specimen Diagnostic for Gram-Negative Pathogens,” Online, Microbiology Spectrum (Washington, DC) 10(6): e0373022; Enko, D., Stelzer I. Bockl M. Derler, B., Schnedl. W. J., Anderssohn, P., Meinitzer, A., and Herrmann, M. 2020. “Comparison of the Diagnostic Performance of Two Automated Urine Sediment Analyzers [the UF-5000 Sysmex and the Cobas® u 701] with Manual Phase-contrast Microscopy,” Clinical Chemistry and Laboratory Medicine (Berlin, Germany) 58(2): 268-273; Yalcinkaya, E., Erman, H., Kirac, E., Serifoglu, A., Aksoy, A., Isman, F. K., and Cekmen, M. B. 2019. Comparative Performance Analysis of Urised 3 and DIRUI FUS-200 Automated Urine Sediment Analyzers and Manual Microscopic Method, Online, Medeniyet Medical Journal (Istanbul, Turkey) 34(3): 244-251; Iñigo, M., Coello, A., Fernandez-Rivas, G., Rivaya, B., Hidalgo, J., Quesada, M. D., and Ausina, V. 2016. “Direct Identification of Urinary Tract Pathogens from Urine Samples, Combining Urine Screening Methods and Matrix-assisted Laser Desorption Ionization-time of Flight Mass Spectrometry,” Online, Journal of Clinical Microbiology (Washington, DC) 54(4): 988-993).

With the manually controllable assistance device shown in FIG. 30, partial retraction or extension of either or both chutes to reduce the rate of collection bag filling where facilities are available warrants careful adjustment. To accomplish this, the knob ends, that is, the distal or port-ward end segments of the control cable inner wires are threaded over the length of 2.0 millimeters. The inner wires are controlled by manually rotating knobs on the surface port which draw in the screws to retract, and move out the screws to advance, the diversion chutes. The thread is cut to a pitch so that one half rotation of either knob moves the inner wire the full 2.0 millimeters. To provide a tactile cue for the halfway point of chute deployment, the knobs are provided with detents.

The precision and durability required of the internal or female thread in the knobs and external or male threads on the control wires is achieved by making the knobs and control wire of titanium alloy Ti-6Al-7Nb (see, for example, Fellah, M., Labaiz, M., Assala, O., Dekhil. L., Taleb. A., Rezag. H., and lost, A. 2014. “Tribological Behavior of Ti-6Al-4V and Ti-6Al-7Nb Alloys for Total Hip Prosthesis,” Advances in Tribology 2014:451387; Bolzoni, L., Hari Babu, N., Ruiz-Navas1, E. M., and Gordo, E. 2013. “Comparison of Microstructure and Properties of Ti-6Al-7Nb Alloy Processed by Different Powder Metallurgy Routes,” pages 161-171 in Imam, M. A., Froes, F. H. and Reddy, R. M., Cost-Affordable Titanium IV. (Key Engineering Materials, 551), Zurich, Switzerland: Trans Tech Publications; Iijima, D., Yoneyama, T., Doi, H., Hamanaka, H., and Kurosaki, N. 2003. “Wear Properties of Ti and Ti-6Al-7Nb Castings for Dental Prostheses,” Biomaterials 24(8): 1519-1524; Semlitsch, M., Weber, H., Streicher, R. M., and Schon, R. 1991. “Joint Prostheses Components of Warm-forged and Surface Treated Ti-6Al-7Nb Alloy,” in German with English abstract at http://europepmc.org/abstract/med/1859861. Biomedizinische Technik [Biomedical Technology] 36(5): 112-119).

When use is to be continuous and permanent, the typically 1.5 millimeter wide by 2.0 millimeter long curved and slightly scooped diversion chutes are advanced once upon placement to fix these in position, no push/pull control cables needed for subsequent retraction and advancement. In a nonadjustable or prosthetic embodiment, the chute is permanently deployed and voiding to an external collection bag is passive or automatic when the pressure actuated switch in the neoureter convergence chamber energizes the impeller. Where the wearer of an adjustable embodiment must be mentally competent, the wearer of the prosthesis need not. As indicated, in situations where ureteral disease recommends interim diversion proximal to the affected segment of the ureter or any lower portion of the urinary tract to allow treatment, medication can be directly targeted through the accessory channel or sideline and jacket to the affected segment.

When a drug or drugs would best be limited to only an affected segment, provided there is a reversal or neutralizing agent for the drug or drugs used, the extent of exposure to these agents can be restricted. If the distal terminus of the segment to be treated is tubular, this is accomplished by placing a second ductus side-entry jacket at the appropriate level to receive the reversal agent through a separate accessory channel. If the terminus is relatively planar or curved, the segment is ended for exposure to a drug or drugs with a nonjacketing side-entry connector. Certain additional capabilities, essentially relevant only for patients unable to adhere to the regimen or self-administer drugs, such as the very young or old, can be further assisted by means of automation.

For example, when the drug insertion apertures in the surface port are covered by a membrane and connected to flat reservoirs positioned subdermally for replenishment by jet injection so that only the urine expulsion line is patent, a fully implanted microcontroller can oversee the delivery of each drug and counteractant as appropriate. Moreover, if malfunctioning, the interruption in timely drug delivery can be telemetrically indicated and radiocasted to the clinic, with responsive adjustment or reprograming accomplished remotely. The ureters are densely innervated and perfused from different sources according to level, so that to transect or sever these at any level without providing a pedicle should not induce atrophy.

At the same time, the accessory channel allows an antimicrobial, anti-inflammatory, antiangiogenic, anesthetic, and/or other drugs as necessary to be directly targeted to the jacket, mainline, and target tissue, thus minimizing if not eliminating the need to reenter other than to remove a temporary jacket or jackets. As shown in FIGS. 6, 11, 12a, 12b, and 12c, of U.S. Pat. No. 11,013,858, a nonjacketing side-entry connector can position a fiberscope along the bladder dome to allow viewing the ureterovesicle, or ureterovesicular, junction, allowing flow into the bladder from the ureters, ureterovesical junction obstructive nephropathy, the presence of diverticula, or the condition of the bladder lining to be directly viewed, which both anatomically and functionally has considerable diagnostic value.

Even when diuresis induces the jet effect (see, for example, Chung-Chieng, Wu 2010. “Ureteric Jet,” Journal of Medical Ultrasound 18,141e146) the more distal or farther down the ureter the takeoff jacket is positioned, the more is intrinsic expulsive pressure likely to be preserved (see, for example, Leung. V. Y., Chu, W. C., Yeung, C. K., and Metreweli. C. 2007. “Doppler Waveforms of the Ureteric Jet: An overview and Implications for the Presence of a Functional Sphincter at the Vesicoureteric Junction,” Pediatric Radiology 37(5): 417-425; Felsen, D., Schulsinger, D., Gross, S. S., Kim, F. Y., Marion, D., and Vaughan, E. D. Jr. 2003. “Renal Hemodynamic and Ureteral Pressure Changes in Response to Ureteral Obstruction: The Role of Nitric Oxide,” Journal of Urology (Philadelphia, Pennsylvania) 169(1): 373-376; Shafik. A. 1998. “Ureteric Profilometry. “A Study of the Ureteric Pressure Profile in the Normal and Pathologic Ureter,” Scandinavian Journal of Urology and Nephrology 32(1): 14-19; Baker, S. M. and Middleton, W. D. 1992. “Color Doppler Sonography of Ureteral Jets in Normal Volunteers: Importance of the Relative Specific Gravity of Urine in the Ureter and Bladder,” American Journal of Roentgenology 159(4): 773-775; Jequier, S., Paltiel, H., and Lafortune, M. 1990. “Ureterovesical Jets in Infants and Children: Duplex and Color Doppler US [ultrasound] Studies,” Radiology 175(2): 349-353; Dubbins, P. A., Kurtz, A. B., Darby, J., and Goldberg, B. B. 1981. “Ureteric Jet Effect: The Echographic Appearance of Urine Entering the Bladder. A Means of Identifying the Bladder Trigone and Assessing Ureteral Function,” Radiology 140(2): 513-515; Henderson, V. E. 1905. “The Factors of the Ureter Pressure,” Journal of Physiology 33(3): 175-188), and the more intrinsic pressure is preserved, the greater will be the number of bodily positions the patient will be able to assume and still void without the need for an assist device in the form of a neoureter convergence chamber impeller.

Thus, to preserve the maximum peristaltic pressure, each jacket is best placed as distad along the ureter as the pathology will allow. Nevertheless, for the patient who restlessly tosses and turns, a pressure responsive assist device in the form of a neoureter convergence chamber impeller is provided. This allows the patient with frequent urination, regardless of cause, to sleep through the night whether supine, prone, on a side, or when changing positions. When automation is applied, the ejection pressure can also be intensified through electrostimulation to amplify the calyceal or pelvic peristalsis.

Free movement is also possible because the diversion line, while made in its intracorporeal length of a fluoropolymer for slipperiness, is connected to a length of highly flexible nonallergenic rubbery tubing such as Saint-Gobain Corporation (La Defense, Courbevoie, Hauts-de-seine, France) Tygon® S-50-HL running from the surface port to the collection bag. Resistance to irreversible deformation kinking also allows the wearer to engage in sports without special concern for sustaining an impact at the outlet leading to the collection bag. Additional information pertaining to the application of vascular valves to the urinary tract is furnished in the section below entitled Description of the Preferred Embodiments of the Invention.

FIGS. 2, 7, and 8 show vascular valves for the diversion of flow, usually of urine from a ureter to a neobladder or collection bag. That in FIG. 2 is switched in position by the operator, and in a urinary prosthesis for which the valve is suitable, can be switched back by a clinician but not the patient. In the urinary tract, synchronization among the one or two valves required is not a factor. By contrast, synchronization is essential in a solid organ transplant such as that of the heart. The solenoid push-configured valve shown in FIG. 7 and bistable linear plunger solenoid pull-configured valve shorn in FIG. 8 are suitable for a sudden bypass, or sudden switched-organ organ transplant under exigent circumstances but not a metered bypass, or sudden switched-organ organ transplant which necessitates continuously variable and immediately responsive control.

During organ transfer, a servovalve such as that shown in FIG. 10A can be controlled to advance gradually in response to sensor inputs such as rejection analytes sent to its controlling microprocessor. The action is spontaneous so that if detected, advancement is paused while remedial medication is directly targeted to the affected sites. The deterrent overcome, the action resumes. If not overcome automatically, intervention by the clinicians is applied. Provided the donor and recipient had been crossmatched, and if necessary, treated preoperatively with tolerance-inducing donor-recipient transplanted tissues and/or medication applied as appropriate, the need to stop the procedure and institute measures beyond the capabilities built into the system as governed by its prescription-program, while unlikely and even if unapparent to the operating team, will be signaled by the system itself.

Actuated simultaneously from the same electrical switch, the solenoid-driven vascular side-entry valves shown in FIGS. 7 and 8 are also suitable for exchanging flow between great vessels when amenable to an extracardiac correction of a total transposition. With a manually controlled mechanical valve, the operator grips the accessory channel stabilization collar 12 to slide the diversion chute and accessory channel forward into the native lumen until the detent 16/17 beneath the chute engage to lock the chute in the forward, or fully deployed position with an audible click. Inaccessible noninvasively, mechanical valves are best limited to applications very unlikely ever to require reversal. By contrast, solenoid-driven valves are reversible when energized with sufficient reverse surge current to overcome the detent, less current needed where the solenoid incorporates a return spring.

Even through mechanically driven valves and solenoid-driven valves without extracorporeal controls are less costly than servovalves, in any application where post-implantation adjustability, much less immediate response to commands from an implanted master controller are beneficial if not essential, the controllability of servovalves makes these preferable for the simplest transplant. This is true if only a few valves are needed, and precisely synchronous deployment of the diversion chutes is not critical. The side-entry opening stopper, or ostium obturator, 30 is permanently is permanently affixed to the diversion chute and no less present in the retracted, or undeployed, position where it is collapsed by constraint inside trepan tube 5 as to obscure the diversion chute.

The outlet of the lower accessory channel is shown as part number 31 below the distal tip of the diversion chute. Whether channeled through the chute as accessory channel 8, relegated to upper accessory channel 8′, or to a separate basic side-entry jacket upstream, of ahead in the flow to the subject or target organ both the upper and lower surfaces of the chute are accessible to medication. Before advancement from the trepan tube into the substrate native lumen, the ostium obturator must be collapsed to be compact as possible, affording sufficient clearance to allow removal of the side-entry tissue plug through use of the suction pump alone, then upon entering the native lumen from which it is not normally withdrawn, self-deploy by unfolding. To spontaneously-unfold when advanced into the native lumen, ostium obturator is made of a shape-memory polymer.

The polymer is soft enough in the elastic state that should it become necessary to remove the diversion device in the absence of a chilling means that would cause it to compact, or fold, the obturator can simply be pulled through the side-entry hole and out into the trepan end-piece with little if any injury to the margin of the side-entry hole. That the trepan is slightly recessed from the luminal border of the side-entry hole reduces any damage to the obturator if any. Upon placement, the diversion jacket, hence, the obturator is at room temperature, or more likely in terms of the shape state changing temperature differential attained to date, pre-chilled, keeping it in the compact rigid state, and when brought to body temperature, unfolds, or deploys, becoming soft and pliant.

Accordingly, highly damped miniature nonsparking plunger solenoid-operated vascular valves such as shown in FIGS. 7 and 8 are useful for sudden switch compound vascular bypass transplantation where there is a low expectation of rejection following the preprocedural administration of immunosuppressives and immune tolerance-inducing drugs and cells, and where synchronous operation of a larger number of valves than can be manually switched at the same instant is essential. The solenoids shown in FIGS. 7 and 8 are schematic in omitting dampers, springs, and other elements without novel significance. When placed, the application of current drives or retracts the plunger (thrust rod, slider, shaft.

As shown here, when readied to be placed, the solenoids are energized to maintain them in the retracted position with the return spring compressed until switched to cut off the current so that the restorative force of the springs ejects the chutes which will then sustain the solenoids in the ejected, vessel-obturated, state without a further need for energy input. In sudden bypass, or sudden switched-organ, solid organ transplantation, adjuvant therapy is by pre- and postprocedural infusion or injection without the interposition of a third midprocedural stage between the two. A triple stage procedure, metered bypass, or sudden switched-organ transplantation allows the controlled progressive release of immune tolerance-inducing substances over the course of time that the diversion chute traverses the distance to complete occlusion of the native lumina and incorporation of the new organ into the recipient.

This transit time is immediately responsive to the detection of an immune response by sensors feeding data to the implanted controlling microprocessor, for example. FIG. 10A shows a linear servomotor-operated vascular valve made to the dimensions of the prospective substrate vessel. When other than the vessels departing and returning to the heart, these will be referred to as miniature or subminiature. Such a servovalves affords continuously variable control of chute deployment and retraction in distance, direction, and speed, any of all of which may be commanded adjusted by the implanted controller at any time during transplantation or postoperatively.

Unique to metered bypass transplantation, midprocedural control consists of moment by moment adjustment of the diversion chutes by the controlling microprocessor responding to physiological sensor feedback in accordance with its prescription-program. Each chute is gradually advanced, and an adverse reaction detected, the advancement is paused and reaction reversal agents released through one or more servovalve accessory channels. Thus, the chutes are controlled in coordination with the release into the lumen of immune tolerance-inducing drugs and the detectable effect these register on the sensor feedback to the controlling microprocessor.

The controller then resumes adjustment of each chute, which may call for continued advancement, partial retraction, holding in place, or even truncating the procedure, largely avoidable by proper matching of the donor and recipient and the administration of tolerance-inducing agents preoperatively. For this reason, the automatic release of agents midprocedurally should not involve doses larger in volume than can be held within small flat reservoirs positioned in the pectoral region. Nevertheless, where the process of tolerance induction must be truncated, the servovalve is capable of sudden and complete retraction, returning the substrate lumen to normal flow. Such action will usually apply equally to all of the valves.

A proportional flow diversion servovalve feedback controlled thus is superior to pulmonary or portal banding, for example, for alleviating hypertension. Any linear motor incorporating or coupled to a positional feedback device in support of proportional-integral-derivative control which can be reduced to subminiature dimensions can be used. In metered bypass, or sudden switched-organ, transplantation, the microprocessor coordinates the positional with the physiological feedback to control the diversion chutes, hence the relative proportion of donor and recipient blood passing through the valves.

FIG. 28 provides an overall view of a nonadjustable embodiment suitable for use as a permanent prosthesis in a patient with an irreversible congenital or traumatic condition involving the lack or loss of the lower, or distal, urinary tract. Where corrective surgery is contemplated, must be deferred to a later date, or has been accomplished, the embodiment is also suitable for use on a temporary basis as a means of diversion away from the affected or operated region, whether unilateral or bilateral. The urine outlet opening 110 through the center of the surface port also serves incurrent use for directly targeting agents to maintain the components and allow the insertion of cabled devices for cleaning and examining the prosthesis.

Urinary diversion jackets such as shown in FIGS. 2, 28, 30, or a diverter such as that shown in FIG. 31, all of which omit the ostium obturator 30 required in any valve suitable for the diversion of blood but which obstruct passage, one can insert diagnostic and therapeutic cabled devices through a pectoral port to look downward through the system or through a pubic port to look upward. Thus, passing the cabled device up through effluent line 110 and passing the device upwards through the diversion jackets allows the clinician to obtain a direct view of the upper or proximal tract by extraurethral pyeloscopy, ureteropyeloscopy, or ureteroscopy extraurethrally, without a topical anesthetic, patient discomfort, or risk of injury.

Compared to a side-entry device, one end-inserted such as those shown in FIGS. 31A and 31B fails to eliminate the need for transection of the vessel which requires clamping or improvising a midprocedural bypass that creates two sites of vascular injury posing the risk of further complications. To directly treat an infection, remove biofilm or crystal, or to obtain a biopsy test sample, the same extraurethral route gives access to every level of the urinary tract outside the kidneys.

FIG. 28 showing a prosthesis and FIG. 30 showing an adjustable embodiment, the accessory channels (service channels, sidelines) omitted appear in the drawings of valves and in FIG. 31, also suitable for use in a prosthesis. Because gravity is working with, rather than against, the flow of fluid maintenance agents, such as a crystal solvent or an antiseptic, into the diversion jackets then down through the rest of the system, and unlike the pelvis, the pectoral region affords space to position drug reservoirs subcutaneously, the accessory channels are best accessed from above rather than below.

In non-prosthetic embodiments, push/pull Bowden cable control knobs for the wearer to adjust mechanical valves, or electrical control knobs to control solenoid-driven or servovalves are best positioned around the urine outflow, or effluent, pipe 110 at the center of the port, for ease of wearer viewing and use placed to a side of the mons pubis. The surface port in a urinary diversion apparatus requiring an external opening to pass urine in any event, the addition of small control knobs does not in itself compel the externalization of the surface port that could otherwise be positioned subdermally. Unless the ureter distal to the jacket is missing, the surface port additionally incorporates a cutaneous, or on the skin, opening into an accessory or service channel which provides a direct passageway into the distal tract for implant maintenance, diagnosis, and therapy.

As shown in FIG. 30, advancement and retraction of either diversion chute is controlled by separate control knobs 153 and 153′. Conventionally, the wearer rotates both knobs the full half-turn between the stop to either side, thus fully advancing or retracting the diversion chutes. The diversion chutes typically in the range of 2.0 to 4.0 millimeters in length, rotation of either control knob by one half-turn ejects or draws in the threaded end segment of the inner push/pull control cable wire to advance or retract its chute by the equivalent amount.

Separate control of either chute rather than of both with a single knob allows renal differential urinalysis by the clinician, and by rotation less than a half-turn, the wearer with convenient facilities to apportion the rate of drainage from either side into the collection bag, reducing the rate of bag filling, or into the lower tract for normal voiding. FIG. 5 provides a longitudinal section and FIG. 6 a cross-sectional view through a wearer-adjustable diversion jacket parallel to the view of a nonadjustable embodiment shown in FIGS. 2 and 3. Miniature push/pull control cables allow the controlling motion to pass from small control knobs at the body surface port through a curved path to the remote jacket to make possible the simple rectilinear motion needed to advance and retract the diversion chute.

In FIG. 30, as it would create problem spots for encrustation, microbial colonization, and thrombus, control cables 28 and 28′ are not run through mainline 9. In the lower left-hand corner of FIG. 5, the distal terminus of cable 28 shows where the push/pull cable used to advance and retract chute 18 is connected to the chute through collar 12 encircling and the accessory channel 8 at the bottom of each diversion jacket. The mechanism which allows a half turn of either know to fully advance or retract the chute to its side is described below in section 6, entitled Description of the Preferred Embodiments of the Invention.

To afford accessory channel 8 clearance to drive diversion chute 18 along its slideway or raceway, FIGS. 5 and 6 show how diversion chute 18, inside trepan tube 5, along the bottom center of the jacket is provided with a slot 27 through the jacket shell 3 and foam 4. To prevent the leakage of urine outside the jacket, these parts, made of a hard and slippery polymer such as gear grade nylon, must be flush fitting. Where foam 4 abuts against the adventitia such as shown in FIG. 5, it is open-celled and permeable to allow medication such as steroidal, antimicrobial, or immunosuppressive to wet the outer surface of the substrate ductus.

FIG. 2, provides a detailed view of a side-entry flow diversion jacket which once set by the operator, remains fixed or nonadjustable, and as such, suitable for a prosthesis. As seen in FIG. 2, the detents in a set-once solenoid-driven diversion jacket for use in a urinary prosthesis consists of a small eminence, or protuberance, 16 along the underside of diversion chute 18 and a depression 17 molded in the surrounding jacket such that engagement of the eminence in the depression with an audible click immobilizes the chute at that extension into the native lumen with sufficient resistance to displacement that moderate force is required to dislodge the eminence from the depression. If necessary, diversion jackets in prostheses can be detent and/or spring overridden by a clinician. FIGS. 31A and 31B show a diverter with accessory channels which can also be used in a prosthesis; however, a non-side entry diverter is structurally limited to diversion.

While flow diverters such as shown in FIGS. 31A and 31B both accomplish diversion by redirecting flow, a diversion jacket differs from a basic side-entry jacket as described in U.S. Pat. No. 11,759,186 in incorporating a chute to divert the flow of blood or urine. The significance of this is that unlike a diverter which is fixed, or ‘dumb, a flow diversion jacket driven by a solenoid with retum spring and no detent allows switching between total and no flow diversion, and one driven by a servovalve allows continuously variable control over the relative proportion of flow leaving an undiverted proportion of flow to continue along its normal path.

Variable controllability is essential for automatic adjustment by a servomotor-driven valve as commanded by an implanted microcontroller or microprocessor in response to sensor feedback. As shown in FIG. 30, in an embodiment which allows the wearer to select between normal voiding and the passing of urine into a collection bag, adjustability is ordinarily by means of push/pull cables controlled by turning knobs on a surface port positioned on the mons pubis. In a bilateral embodiment, both knobs can be on the same port or each can be on separate ports to either side of the mons pubis. Solenoid-driven diversion jackets are readily producible but require disposable or rechargeable button cells surrounding the urine outflow pipe 110 as are the injection opening in FIG. 28 and the control knobs in FIG. 30.

Both a diverter such as shown in FIGS. 31A and 31B and a diversion jacket enable the creation of fluid shunts and bypasses using even small caliber synthetic tubing without the need to harvest a native ductus such as a vein and in so doing, create a preliminary site for the development of complications with the potential to prove serious. Both incorporate accessory channels which allow the direct pipeline targeting of anticoagulant and crystal dissolving agents into their synthetic lumen and drugs into the substrate native lumen. Not requiring adjustability, both diverters and solenoid-drive diversion jackets are suitable for use in a prosthesis such as that shown in FIG. 28 to replace the lower urinary tract.

By contrast, in an embodiment for use on an intermittent or discretionary basis, diversion must be selectable, making a deployable and retractable diversion chute and thus a diversion jacket rather than a fixed diverter necessary. An accessory channel allows the direct targeting of antimicrobials, anticoagulants when blood and crystal encrustation counteractants when urine is diverted without which smaller catheters invariably become fouled and/or clogged. Synthetic shunts and bypasses have been used for decades, but this factor has prevented the use of catheters to like purpose over the same period.

In a urinary jacket or valve, the integral drug delivery accessory channel, seen as part number 8 in the drawing figures, continues toward the ureteral lumen through a groove running centrally along the underside and extending to the distal tip of the chute to emit through a small opening 31 along the underside of the chute 18, allowing medication to be dripped into the substrate vessel or ureter. FIG. 3 provides a detailed cross-sectional view of the diversion chute shown through trepan tube 5 along line A-A′ in FIG. 2. In a nonadjustable or fixed prosthetic embodiment, the chute is never retracted as would allow urine to continue through a functioning lower urinary tract.

The design of the side-entry diversion jackets and valves shown allows the diversion chute to be advanced into and retracted out of the trepan piece without abrasive contact of the chute against a surrounding edge of unprotected tissue. The moisture barrier-protected foam enveloping the adventitia (outer surface) of the ureter or artery is continuous entirely about the trepan, assuring leak free operation. While an adjustable embodiment must continue to be controlled remotely after placement, a prosthesis need be set only at the time of placement when the operator has direct access to the treatment site. In a vascular embodiment, a side-entry hole stopper, or ostium obturator, at the front end of the chute is necessary to prevent the flow of blood back into the trepan tube.

Before the diversion jacket or valve is placed, the ostium obturator is collapsed with the chute retracted inside the trepan tube, as depicted in FIGS. 78, and 10A. When the chute is advanced into the native lumen, full extension of the chute for full diversion of flow is with the ostium obturator flush against the opposing wall of the native lumen. Retraction of the chute so that flow is not diverted places the ostium obturator flush against to fully cover over the side-entry opening as a stopper. Thus, once deployed, at no time does the ostium obturator reassure its preplacement position inside the trepan tube so that outflow would be obstructed when the obturator is positioned flush against the opposite or far side of the ductus. This is shown in FIG. 5, where a dashed line represents the ostium obturator as covering over the opening into the trepan tube with the chute retracted, and solid lines show the chute as fully deployed. Used to drain urine continuously, a urinary prosthesis requires no ostium obturator.

Upon placement, to allow sufficient cross-sectional clearance to allow the tissue plug to be removed, the ostium obturator is best sufficiently pliant so that the suction hose will be able to withdraw the plug past it without requiring a level of vacuum pressure as would drive the trepan against the opposing wall of the native lumen prevented by a limit switch on the pump. Should the plug catch on the obturator, the clamp shown in FIG. 4 will allow the suction hose to be disconnected and a miniature dental pick used to pull out the plug. Clamping thus is harmless with most arteries but not the carotids where even a momentary interruption in flow can provoke a cerebral infarction, or in coronaries where a myocardial infarction might ensue. The coronaries have been known to spasm even when another nearby artery has been accessed. 7

This eventuality can be averted through the upstream administration of antispasmic agents such as prostacyclin, nitroglycerin, isosorbide dinitrate, nimodipine, verapamil, diltiazem, nifedipine, propranolol, an L-type calcium channel blocker, or β-blocker, each according to its onset of action and effective duration. Extension 162 and retention barbs 158 necessarily covered over by a layer of sugar or frozen butter when a straight ductus coupling or stent version of the diverter without the bend is positioned transluminally, the risk of vasospasm is dispelled through the administration of an antispasmic such as one of those just specified. Adverse side effects are generally associated with continued use over a period much greater than that required, and an infarction should not ensue within the time needed to retrieve the side-entry plug and place the valve.

A diverter such as that shown in FIGS. 31A and 31B allows no portion of flow to continue along its normal path and is not intended for use in carotids or coronaries. As may be noted in FIG. 28, because it always diverts urine away from either or both ureters, a nonadjustable embodiment such as used in a urinary voiding prosthesis requires no side-entry hole obturator. In an adjustable embodiment such as that shown in FIG. 30, once advanced into the native lumen, the obturator moves between two positions, either flush against the far wall of the ductus, here a ureter, for fully diverted flow or flush against the near wall, thus clearing the native lumen for fully normal, or undiverted, flow. In either position it must comply in concave or convex curvature to the luminal wall.

Rotating the control knob in the surface port retracts the obturator so that it covers over the side-entry hole, sealing off the diversion route limiting the flow of urine to the lower tract, while rotating the knob in the opposite direction advances the obturator against the urothelium opposite the side-entry hole. Should medical or surgical treatment allow the lower tract to recover, the obturator should be sufficiently elastic at body temperature to allow its removal by pulling it out through the side-entry hole with little if any injury to the ureter.

To allow healing with closure of the small side-entry hole, a piece of absorbable tape is placed over it to both seal the ductus and appose its sides, thus prompting primary healing, or healing by first intention. Should extended positioning of the obturator in flush relation to the urothelium at either side provoke an adverse tissue reaction, the accessory channel affording access is used to deliver a counteractant, usually steroidal to include an anesthetic. A basic side-entry jacket positioned upstream to automatically release medication on a scheduled basis can be placed at the same time the voiding assist device is implanted. The automatic release of drugs from subcutaneously, or subdermally, positioned small flat reservoirs is addressed in U.S. Pat. Nos. 11,013,858 and 11,759,186.

The diversion chute, 18 in the accompanying drawing figures, is sized according to the internal diameter of the substrate ductus. The lumen will normally vary between individuals and along its length between 2.0 to 5.0 millimeters in diameter and when pressed flat, the chute, made of a suitable polymer sufficiently plasticized to impart a soft rubbery character, will generally vary between 5.0 to 10 millimeters in length. When advanced into the ureteral lumen, the distal tip of the chute and its sides must flush fit conform to the internal contour of the lumen. Any plasticizer residue must be fully removed. Running along the bottom center of the chute is the continuation of the catheteric drugline which upon reaching the chute becomes an accessory channel.

Accessory channels coursing thus or entering the diversion jacket or valve separately are available to directly pipeline target drugs and diversion system maintenance agents to the jacket. System maintenance agents can be automatically delivered according to the microcontroller prescription-program schedule. Disease associated analyte sensors can be incorporated into the diversion jackets or the diverter shown in FIGS. 31A and 31B or positioned separately. While ordinarily used for urine outflow, the effluent pipe 110 in FIGS. 28 and 30 can be used for drug inflow. Use of this path to delivery drugs depends upon which portion or portions of the lower urinary tract remain intact.

In a user controlled embodiment such as the voiding assist system shown in FIG. 30, the entire lower tract is intact. While a bladder infection is much reduced where the urethra is missing, retrograde use of the effluent pipe 110 to spray the bladder interior with a biofilm-breaking antiseptic can eliminate the need for an oral antibiotic. The accessory channel running along the bottom center of the chute allows drugs to be delivered into the urinary tract situated below the chute. Retrograde delivery of drugs through the mainline and trepan tube coursing through the side stem 19 allows drugs to be forced up through the ureter and into the renal pelvis and calyxes, the use of contrast allowing the level reached to be viewed.

In a prosthesis such as that shown in FIG. 28, the free or stump end of the ureter with a diversion jacket as shown extends down somewhat before it is cut and completely tied off. In contrast, when the prosthesis diverts urine with a diverter as shown in FIGS. 31A and 31B, the diverter defines the distal terminus of the ureter. Thus, with a diversion jacket, urine is prevented from entering the irregular space beneath chute 18, whereas with a diverter, no such space is present. The chute in a prosthesis is fixed in the fully extended, or deployed, position and therefore omits means of adjustment such as the push/pull control cables shown in FIG. 30.

When the urothelium is normal and the lower tract expected to heal so that no part of it is resected, a diversion jacket offers the advantage of operator-discretionary return to undiverted flow while allowing the prosthesis to remain in place during a follow-up period for ‘watchful waiting,’ Where the lower tract has been surgically removed such as to prevent the spread of cancer or so damaged as not to be practicably reparable in an elderly patient, for example, a diverter such as shown in FIGS. 31A and 31B is less expensive than a diversion jacket.

In the adjustable embodiment shown in FIG. 30, control over the extent of chute extension by push/pull control cables avoids the greater cost of servomotors. Snug apposition against the surrounding lumen wall is achieved without the need for precise adjustment during placement by making the chute of a highly pliant thin soft rubbery material having a feathered surrounding edge molded with a slight upturn. The surrounding edge of the chute therefore continues to bend upward in compliance with minimal resistance posed by the surrounding lumen wall.

Shown in context in FIGS. 28 and 30, FIG. 29 provides a perspectival section view through a neoureter convergence or confluence chamber 145, into which each jacket outlet drainage catheter, or neoureter, empties. Here synthetic components are substituted for a neobladder and line terminating in a stoma surgically constructed from gut, which is unadapted to and degenerates in contact with urine, so that a primary operation with its complications and adverse sequelae are avoided. Depending upon the length and peristaltic sufficiency of the vestigial ureter above the level of the diversion jacket 143 and/or diversion jacket 143′ when the patient is upright, the pressure of urine upon entering the ureteral side-entry jacket supported by gravity should be sufficient to expel urine out through neoureter 144 and/or 144′ and the surface port into the collection bag.

With urge sensation lacking, this passive process may be satisfactory to the patient who does not mind wearing a collection bag at all times, allowing voiding directly from the ureters through the effluent pipe 110 into the collection bag 148. However, even the patient without a bladder and urge sensation may prefer to avoid the need for a collection bag. For such patients, a means for the temporary storage of urine and signaling that the storage area is full allows voiding directly into a bathroom receptacle without the need for a collection bag. Where the urethra is intact and can be accessed to place a basic side-entry jacket or diverter connecting it to a synthetic neobladder or neoureter confluence chamber, normal voiding should be attainable.

In most cases, urethral emission will prove impracticable so that voiding will be through the effluent pipe 110 out surface port 104 into a bathroom receptacle. Without urge sensation or voluntary control, retention by the internal and external sphincters will not prevent the uncontrolled release of urine. With a fully passive system leading to a collection bag, this is unobjectionable, but in a system intended to allow voluntary voiding, means for signaling to the wearer that the confluence chamber is full and means for controlling its emptying are imperative.

As shown in FIG. 29, the confluence chamber incorporates an impeller 150 and pressure actuated switch 152, in addition to a continent flap at the outlet of the confluence chamber (not shown) functioning as a synthetic neobladder. The impeller compensates for any loss in peristalsis. When the pressure in the chamber exceeds a threshold level nearing that associated with reflux, the assist device signals the need to expel the chamber contents. The preferred means for signaling is vibratory as is standard in smartphones. The vibration can be by an eccentric rotation mechanism internal to the confluence chamber or a signal radiocasted to a smartphone.

An effective urinary assist prosthesis or assist device must accommodate several variables that distinguish potential users. These variables cover intact or missing anatomy and urge sensation. For example, the ability to always void voluntarily while erect will be true of the user with intact urge sensation provided with a controllable or adjustable embodiment for use during the day. Without urge sensation, this will not be true while the user is asleep. To cover all of these eventualities except in some cases where drainage is passive directly into a collection bag, both controllable and most fully prosthetic systems include a synthetic neobladder, or neoureter confluence chamber.

Shown in FIG. 29, the confluence chamber incorporates a pressure actuated switch 152 and an impeller connected to a battery housed in the surface port to empty the chamber into a collection bag whenever the user, usually while sleeping, does not notice the vibratory signal indicating that the collection chamber is full so that a trip to the bathroom is needed. That is, in some cases, a collection bag is worn at night even when the patient would otherwise not require one. As shown, the jacket to either side receives a single accessory channel 8; however, where for example, drugs are best administered separately or one accessory channel should be reserved for drugs and another for maintenance of the apparatus, a second accessory channel 8′ is provided.

Additional druglines leading directly from a surface port where these can be injected or from an intervening implanted drug or maintenance agent reservoir can be provided for either or both jackets, different entry point therefor appearing in the drawing figures of valves. The wires connecting the pressure actuated switch 152 to the battery are passed from one or more single use or rechargeable button cells position about the central effluent pipe 110 if present on the surface port as would be subcutaneous drug entry opening or control knobs. Pressure switch 152 actuates impeller 150 when the chamber reaches the threshold fill level or there is an increase in pressure that would induce reflux. The fan-shaped impeller allows flow past it, and forcible ejection when actuated by switch 152.

When the power source is not located within the impeller housing 150 for transcutaneous recharging, electrical connection of the power source, here a battery, to pressure actuated switch 152 can be through a separate suitably insulated wire or coursed (run, ‘snaked’) through a dedicated catheter entered through a leak-proof biocompatible, grommet, such as one of nylon, to the switch with terminal connection also protected by a similar leak-proof grommet. The pressure actuated switch is a microswitch comprised of parallel conductive leaves, one of which is bonded to the side of the impeller. The conductive leaves are separated by a leaf spring which under the threshold pressure, flattens, bringing the leaf above and below the spring into contact.

In all cases, electrical and fluid lines must be routed to avoid the possibility of strangling intervening tissue regardless of whether the patient is standing, sitting, or recumbent. When this cannot be accomplished with confidence, fluid and electrical lines connected to a surface port which does not allow this must be reconnected to another surface port positioned to allow a safer route.

Involving additional expense, the avoidance of hardwiring through the use of radio remote, such as Bluetooth, connections should be reserved for patients with multiple comorbidities in whom the use of several hard wire connections would best be avoided.

In an intractable stone former having been treated with no more than partial success, to avert the risk of reflux into the native ureter of a stone solvent such as potassium citrate, magnesium potassium citrate, allopurinol, or sodium bicarbonate at a concentration higher than would be allowed to contact unaffected native tissue, the solvent can be led directly from a subcutaneous drug or agent opening in a surface port to the diversion jacket to flow down into the convergence chamber, thus preventing encrustation and obstruction at the chamber outlet. Sites of stone accretion at higher levels in the kidney, calyces, or pelves can be directly pipeline targeted with the aid of one or more nonjacketing side-entry connectors as described and shown in U.S. Pat. No. 11,013,858 of like title, FIGS. 6, 11, 13A, and 13B.

Means for increasing the force of expulsion include electrostimulation by a neuromodulator implant and the use of a diuretic to stimulate calyceal peristalsis. FIGS. 26A and 26B provide sectional views through a subcutaneous pectoral surface port suitable for use with the nonadjustable, or prosthetic, embodiment shown in FIG. 28. This surface port differs from a port for an adjustable, or uwer-controlled, embodiment in omitting small push/pull control cable knobs of electrical switches to advance or retract the diversion chute. A suitable port for use of a controllable embodiment is positioned to a side of the mons pubis for ease of viewability and use is shown in FIG. 26C. Such a port retains drug entry opening subcutaneously and combines these with a urine effluent pipe 110 which passes through the port to open outside the body at the port center. The end of pipe 110 is protected by a cap containing a material such as gauze which can be wetted before replacing the cap in position.

As indicated, when more than one opening is subcutaneous, or subdermal, and each calls for a different drug or agent, small tattoo marks shown in FIGS. 17 and 27C allow identification of the different openings and thus indicate the proper alignment of a multiple reservoir replenishing injection head such as those shown in FIGS. 27A and 27B. With one or more implanted drug reservoirs, the administration of each drug or apparatus maintenance agent such as a thrombolytic or crystal solvent can be controlled by a microcontroller in single morbidity or less complex comorbidity, or by a microprocessor in more complex comorbidity.

With patients who are mentally disturbed, impaired, or too young to be entrusted with an on-the-skin, or cutaneous, surface port, especially one that includes one or more controls, ports are positioned so that only an adult or clinician can gain access. A port with drug openings is best positioned subcutaneously, and one with one or more controls between the shoulder blades with as little protrusion or discomfort when supine or resting against the back of a chair as possible. The port is best backed with a thinner cushion of the type used to alleviate joint pain in arthritis. In order avoid the need to place the surface port on the skin rather than subdermally only to house a battery for quick replacement, a transdermal or transcutaneous antenna can be implanted to allow an implanted battery to be recharged by transdermal energy transfer.

While it is improbable that a sufficient volume of a drug cannot be stored within an implantable reservoir so that replenishment is required at inconveniently frequent intervals, each time with some injury to the skin, a volume of a drug or agent not accommodated can justify the placement of a second reservoir. Both can be filled through a single opening in the port for delivery through the same accessory channel. Where the need exists to insert a cabled device is frequent as would require a small incision to gain access to the original or root-site of disease, a port such as that shown in FIG. 26C is used.

While more often controlled electronically, remotely controlled valves which can differentially apportion the flow of blood, drugs, or urine through either or two ductus with continuous variability between that fraction diverted and that fraction allowed to continue through the native lumen can be adjusted manually. As shown at the lower left in FIG., 30, one means of control uses push/pull, or Bowden cables adjusted by turning rotary control knobs placed on a small, secure, and infection-resistant port at the body surface. Unlike a fully automated embodiment, this assumes a competent adult patient.

As shown in U.S. Pat. No. 11,389,171, entitled Integrated System for the Infixion and Retrieval of Implants, when accessory channels are used to release a drug into the stream or streams as apportioned for enhanced uptake along the segment endothelium or urothelium, or adhesion along the internal surface of the tubing or catheter, a drug or agent without intrinsic affinity as iodine for the thyroid gland, for example, can be bound to a superparamagnetic carrier making it magnetically susceptible to an elongated magnetized jacket to surround the segment to be selectively medicated or coated thus. For use on native ductus, such jackets incorporate multiple means for preventing the atrophy of the substrate ductus.

The insertion of a cabled device such as a fine fiber scope or laser to examine or service the urinary tract or the interior of the apparatus such as shown in FIGS. 28 and 30 are present and conventional access is not possible is through the urine outlet opening into the effluent pipe 110 at the center of the surface port shown in FIG. 26C. FIG. 30 shows the overall plan of an adjustable, or user-controlled, embodiment suitable for use on an intermittent basis to prevent the need to void when the wearer is indisposed or would be subject to frequent interruption during sleep. Intermittent use embodiments accommodate both daytime use for the wearer with access to bathroom facilities and able to void while standing, and before going to bed can put on a collection bag for passive drainage without being aroused.

### Infection of the Urinary Tract Before and After Replacement

FIGS. 28 thru 30 show a prosthesis for replacement of the lower urinary tract in its entirety. Except in a case of carcinoma, where to preclude metastasis to neighboring structures, it is often necessary to perform a complete pelvic evisceration, not all portions of the tract such as the lower portions of the native ureters and/or the bladder need always be replaced.

A defective bladder can be surgically reconstructed with autologous or engineered tissue, replaced in whole or part with a prosthesis called a neobladder, or a transplant (see, for example, Jundzill, A., Witmanowski, H., Zary-Sikorska, E., Adamowicz, J., Bodnar, M., and 7 others 2021. “A New Heterotopic Vascularized Model of Total Urinary Bladder Transplantation in a Rat Model,” Online, Nature Research Scientific Reports 11(1): 3775; Wang, X., Zhang, F., and Liao, L. 2021. “Current Applications and Future Directions of Bioengineering Approaches for Bladder Augmentation and Reconstruction,” Online, Frontiers in Surgery 8:664404; El-Taji, O. M., Khattak, A. Q., and Hussain, S. A. 2015. “Bladder Reconstruction: The Past, Present, and Future,” Online, Oncology Letters 10(1): 3-10; Adamowicz, J., Kowalczyk, T., and Drewa, T. 2013. “Tissue Engineering of Urinary Bladder-Current State of Art nd Future Perspectives,” Online, Central European Journal of Urology 66(2): 202-206; Sawada, H., Esaki M., Sheng, H.<., Kita, A., and Yoshida, T. 2004. “Transplantation of the Urinary Bladder and Other Organs in the Subcutaneous Tissue Induces Cyst Formation and Epithelialization: Its Potential Usefulness in Regenerative Medicine,” Wound Repair and Regeneration 12(1): 30-37).

Accordingly, druglines 8 and 8′ in FIGS. 28 and 30 then give directly pipe-targeted access to parts of the tract that have been preserved, transplanted tissue requiring immunosuppressive support, prosthetic parts a crystal solvent, and both antimicrobials. Were the native bladder preserved, the drugline of druglines would be connected to it by means of nonjacketing side-entry connectors as described in U.S. Pat. No. 11,013,858, entitled Nonjacketing Side-entry Connectors and Prosthetic Disorder Response Systems, and shorn in FIGS. 12A thru 12C.

Urinary tract infection surveys cover numerous if not all pathogens that infect the urinary tract (see, for example, Bono, M. J., Leslie, S. W., and Reygaert, W. C. 2022. “Urinary Tract Infection,” Online, Treasure Island, Florida: StatPearls Publishing Co.; Li, R. and Leslie, S. W. 2022. “Cystitis,” Online, Treasure Island, Florida: StatPearls Publishing Co.; Sabih, A. and Leslie, S. W. 2022. “Complicated Urinary Tract Infections,” Online, Treasure Island, Florida: StatPearls Publishing Co.; O'Grady, M. C., Barry, L., Corcoran, G. D., Hooton, C., Sleator, R. D., and Lucey, B. 2019 “Empirical Treatment of Urinary Tract Infections: How Rational are Our Guidelines?,” Online, Journal of Antimicrobial Chemotherapy 74(1): 214-217; Ganzeboom, K. M. J., Uijen, A. A., Teunissen, D. T. A. M., Assendelft, W. J. J., Peters, H. J. G., Hautvast, J. L. A., and van Jaarsveld, C. H. M. 2018. “Urine Cultures and Antibiotics for Urinary Tract Infection in Dutch General Practice,” Online, Primary Health Care Research and Development 20e41; Flores-Mireles, A. L., Walker, J. N., Caparon, M. and Hultgren, S. J. 2015. “Urinary Tract Infections: Epidemiology, Mechanisms of Infection, and Treatment Options,” Online, Nature Reviews. Microbiology 13(5): 269-284).

Bacterial infections of the urinary tract before and after transplantation are those most common and treated with nitrofurantoin, amoxicillin/clavulanic acid, cephalexin, trimethoprim/sulfamethoxazole, nitrofurantoin, fosfomycin, cephtriaxone, ciprofloxacin (use since reduced due to weakening of ligaments), levofloxacin, or cephalosporins.

Antivirals used include cidofovir, fluoroquinolone, nitazoxanide, and intravenous immunoglobulin, all potentially nephrotoxic (see, for example, Luciani, L. G. and Mattevi, D. 2022. “Urinary Tract Infections: Virus,” Online, Encyclopedia of Infection and Immunity 32-43; Suarez Fernandez, M. L., Ridao Cano, N., Alvarez Santamarta, L., Gago Fraile, M., Blake, O., and Diaz Corte, C. 2021. “A Current Review of the Etiology, Clinical Features, and Diatnosis of Urinary Tract Infection in Renal Transplant Patients,” Online, MDPI [Multidisciplinary Digital Publishing Institute] Diagnostics (Basel, Switzerland) 11(8): 1456; Hatlen, T., Mroch, H., Tuttle, K., Ojogho, O., Rooney, M., Desmond, S., and Bani-Hani, S. 2017. “Disseminated Adenovirus Nephritis after Kidney Transplantation,” Online, Kidney International Reports 3(1): 19-23; Heyns, C. F., Groeneveld, A. E., and Sigarroa, N. B. 2009. “Urologic Complications of HIV and AIDS,” Online, Nature Clinical Practice Urology 7(4): 178). Viral infections often necessitate reducing immunosuppressive medication risking rejection of the graft organ.

Mycotic, or fungal, infections of the urinary tract are treated with amphotericin, fluconazole, or flucytosine (see, for example, Odabasi, Z. and Mert, A. 2020. “Candida Urinary Tract Infections in Adults,” World Journal of Urology 38(11): 2699-2707; Thomas, L. and Tracy, C. R. 2015. “Treatment of Fungal Urinary Tract Infection,” Urologie Clinics of North America 42(4): 473-483). Systemic antimycotics, or antifungals, include terbinafine, griseofulvin, fluconazole, and ketoconazole.

Parasitic infections of the urinary tract such as protozoan and helminthic are caused by numerous pathogens (see, for example, Khurana, U., Majumdar, K., Kapoor, N., Joshi, D. Goel, G., Sharma, T., and Biswas, D. 2018. “Spectrum of Parasitic Infections in Centrifuged Urine Sediments from a Newly Developed Tertiary Care Centre in Central India,” Online, Journal of Parasitic Diseases 42(4): 608-615). Treatment for protozoal infection is with eflornithine, suramin, melarsoprol, nifurtimox, nitazoxanide, metronidazole, tinidazole, nifuratel, quinine, hydroxychloroquine, or sodium stibogluconate. Treatment for helminthiasis is with a dewormer, vermicide, or anthelminthic such as albendazole, mebendazole, nitazoxanide, praziquantel, or ivermectin.

For release by an implanted prosthetic disorder response system, a drug is dispersed in water. Drugs such as antibiotics and antivirals that require passage through the liver are initially prepared to have already been metabolized thus. Rifampicin can be applied topically (see, for example, Shiels, S. M., Tennent, D. J., and Wenke, J. C. 2018. “Topical Rifampin Powder for Orthopaedic Trauma Part 1: Rifampin Powder Reduces Recalcitrant Infection in a Delayed Treatment Musculoskeletal Trauma Model,” Online, Journal of Orthopaedic Research 36(12): 3136-3141; Shiels, S. M., Tennent, D. J., Lofgren, A. L., and Wenke, J. C. 2018. “Topical Rifampin Powder for Orthopaedic Trauma Part II: Topical Rifampin Allows for Spontaneous Bone Healing in Sterile and Contaminated Wounds,” Online, Journal of Orthopaedic Research 36(12): 3142-3150; Xiong, L., Pan, Q., Jin, G., Xu, Y., and Hirche, C. 2014. “Topical Intrawound Application of Vancomycin Powder in Addition to Intravenous Administration of Antibiotics: A Meta-analysis on the Deep Infection after Spinal Surgeries,” Online, Orthopaedics and Traumatology: Surgery and Research 100(7): 785-789).

Inadequate delivery of rifampicin, or rifampin, and isoniazid to eradicate urinary tuberculosis due to dose or prescription schedule noncompliance is ameliorated when the drugs are automatically administered and directly targeted in higher than systemic concentration through ductus or diversion side-entry jackets to the expressed site or sites of the disease, such as tuberculosis of the arteries supplying the affected tissue. Whether the apparatus described can be placed to alleviate ductal hemorrhage, such as ureteral, without surgical intervention depends upon the extent of the breach. If small, the jacket is placed over the affected segment to hold the surrounding tissue into juxtaposed relation and contain the bleeding. Regardless of this mechanical approach, post-transplantation infection that leads to hemorrhage can be treated, as addressed below.

Similarly, when the blood or urine, for example, is infected by a pathogen which is resistant to antimicrobials introduced at the maximum dose which might be circulated safely, a safely circulated systemic dose is administered, and whether due to disease or recent surgery, a more susceptible segment is isolated for receipt of a dose high enough to affect the pathogen more effectively. As will be addressed, the same applies when urine should be kept away from a diseased segment of a ureter, when the urine contains a pathogen, and under normal conditions when the pH is as low as 5.5. This is accomplished by adjusting the constituents of the urine around the more susceptible segment. The same applied along the vascular tree when a segment of a vessel is vulnerable as to recommend protecting it from the passing blood.

To do this, a conventional side-entry jacket with downwardly inclined trepan accessory channel end piece or an inverted adjustable diversion side-entry jacket with slightly deployed chute is positioned at the upper end of the segment to be more heavily medicated, and a jacket oriented reciprocally to these is positioned at the lower end of the segment. The antimicrobial or antimicrobials are then delivered into the affected segment through the accessory channel of the upper jacket from its opening in the body surface port. If uptake of the antimicrobial or antimicrobials while traversing the affective segment is insufficient, a reversal agent or counteractant in sufficient concentration to reduce the antimicrobial outflow to the level that might be allowed to continue through the general circulation is delivered through the accessory channel of the lower jacket.

If no reversal agent is available, then the lower jacket is provided with one or more electromagnets and the antimicrobial or antimicrobials administered in the form of a ferrofluid wherein these are bonded to a susceptible carrier. When the bladder is infected, the lower side-entry device is a nonjacketing side-entry connected positioned beside the trigone, caudally enough to include all but the floor of the bladder. If persistency of urge results, this is remedied by delivering medication through the connector to reduce the discharge rate of the stretch sensors in the trigone. Drugs to alleviate frequent or unremitting urgency, or urge urinary incontinence, include acetylcholine antagonists, or (anticholinergic agents.

Provided interacting drugs are not delivered together but the anticholinergic is singled out for targeted delivery as described, the adverse drug interactions of the anticholinergic will be avoided as will the high rate of drug noncompliant discontinuation (see, for example, Demaagd, G. A. and Davenport. T. C. 2012. “Management of Urinary Incontinence,” PT [Pharmacy and Therapeutics] 37(6): 345H-361H). If excessive urgency and frequency had been the result of a resistant infection before cured and no condition remains to recommend that the side-entry connector remain, it is removed, especially if it causes discomfort. However, if the patient is prone to contract lower tract infections and the connector does not cause discomfort, it along with the other prosthetic voiding components should remain in place for future use.

The use of an array of permanent magnets positioned along the length of the jacket which have been magnetized to project progressively higher strength fields normal to the luminal axis in the antegrade direction is addressed in U.S. Pat. No. 11,389,171, entitled Integrated System for the Infixion and Retrieval of Implants. The detention of a magnetically susceptible carried nanoparticulate, that is, a ferrofluid-borne carried superparamagnetic nanoparticulate drug, at the affected tissue is with a side-entry jacket or jackets incorporating permanent magnets or an electromagnet, and to draw the drug into the diseased tissue is by increasing the field strength of the electromagnet or electromagnets.

Whether preceded by a pulmonary thrombectomy or placement of a vena cava filter, medicinal prevention of a deep vein thrombus and pulmonary embolism, for example, is by directly targeting warfarin and/or heparin, and if resistant as to have already formed clot, a thrombolytic such as tissue plasminogen activator, streptokinase, or urokinase in higher than systemic concentration to the bronchial arteries, with background systemic dosing to prevent deep vein thrombosis in the lower legs.

As will be made clear in addressing direct recipient to donor bloodstream switching in heart transplantation, whenever possible, the donor organ is not harvested, unless transported with the aid of an Organ Care System® (TransMedics, Incorporated, Andover, Massachusetts) machine (see below), not kept in a frigid cardioplegic solution for hours, and not subjected to the shock of abrupt reperfusion and its potential consequences, all of which impair it prior to transplantation. Instead, harvesting succeeds the immediate transfer of perfusion by the machine-assisted donor to the bloodstream of the recipient. Nevertheless, side-entry diversion jackets used as vascular valves offer advantages over clamping and the systemic dispersal of drugs, for example, in the present context whereby jackets must continue to be positioned along donor organ stumps prior to harvesting.

Given the imposition of this constraint, provided the nephrotoxicity if any of the immunosuppressant drug is ameliorated when administered as a nanoparticulate with uptake at the original or root-site of disease, or if more conventional, can be counteracted, repositioning of a nonjacketing side-entry connector at the pelvis of a transplant kidney prior to harvesting allows the direct targeting of rejection-counteractive drugs such as the macrolide cyclosporin A (ciclosporin, cyclosporine), mammalian targets of rapamycin (sirolimus), tacrolimus, or everolimus, for example, monoclonal antibody, such as faralimomab, otelixizumab, or atorolimumab, or polyclonal antibody, such as antithymocyte globulin, to the kidney with allogeneic hematopoietic bone marrow transplant or stem cells systemically circulated in the recipient (see, for example, Mccarthy, D. P., Yap, J. W., Harp. C. T., Song. W. K., Chen, J., Pearson, R. M., Miller, S. D., and Shea. L. D. 2017. “An Antigen-encapsulating Nanoparticle Platform for TH1/17 [T helper cell type 1/17] Immune Tolerance Therapy,” Nanomedicine 13(1): 191-200; Koo. T. and Kim, J. S. 2017. “Therapeutic Applications of CRISPR RNA-guided Genome Editing,” Briefings in Functional Genomics 16(1): 38-45; Kim, E. J., Kang. K. H., and Ju. J. H. 2017. “CRISPR [clustered regularly interspaced short palindromic repeats]-Cas [CRISPR-associated protein 9 nuclease]9: A Promising Tool for Gene Editing on Induced Pluripotent Stem Cells,” Korean Journal of Internal Medicine 32(1): 42-61; Fakoya, A. O. 2017. “New Delivery Systems of Stem Cells for Vascular Regeneration in Ischemia,” Online, Frontiers in Cardiovascular Medicine 4:7; Kang, N., Toyofuku, W. M., Yang, X., and Scott, M. D. 2017. “Inhibition of Allogeneic Cytotoxic T Cell (CD8+) [cluster of differentiation 8] Proliferation via Polymer-induced Treg [T regulatory] (CD4+) [cluster of differentiation 4] Cells,” Acta Biomaterialia pii: S1742-7061(17) 30263-30265; Hlavaty, K. A., Mccarthy, D. P., Saito, E., Yap. W. T., Miller, S. D., and Shea. L. D. 2016. “Tolerance Induction Using Nanoparticles Bearing H Y Peptides [the male-specific histocompatibility antigen, H-Y] in Bone Marrow Transplantation,” Biomaterials 76:1-10; Dobrovolskaia, M. A., Shurin, M., and Shvedova, A. A. 2016. “Current Understanding of Interactions Between Nanoparticles and the Immune System,” Toxicology and Applied Pharmacology 299:78-89; Najafi-Hajivar, S., Zakeri-Milani, P., Mohammadi, H., Niazi, M., Soleymani-Goloujeh, M., Baradaran, B., and Valizadeh, H. 2016. Najafi-Hajivar, S., Zakeri-Milani. P., Mohammadi, H., Niazi, M., Soleymani-Goloujeh, M., Baradaran, B., and Valizadeh, H. 2016. “Overview on Experimental Models of Interactions between Nanoparticles and the Immune System,” Biomedicine and Pharmacotherapy 83:1365-1378; Savic, N. and Schwank, G. 2016. “Advances in Therapeutic CRISPR/Cas9 Genome Editing,” Translational Research 168:15-21; Abrahimi. P., Qin, L., Chang. W. G., Bothwell, A. L., Tellides, G., Saltzman, W. M., and Pober, J. S. 2016. “Blocking MHC [major histocompatibility complex] Class II on Human Endothelium Mitigates Acute Rejection,” JCI [Journal of Clinical Investigation] Insight 1(1) 1-16. pii: e85293; Borges. T. J., Murakami, N., and Riella, L. V. 2016. “Current Status of Alloimmunity,” Current Opinion in Nephrology and Hypertension 25(6): 556-562 El-Kashef. D. H., El-Kenawi. A. E., Sudde k. G. M., and Salem, H. A. 2016. “Allicin Ameliorates Kidney Function and Urinary Bladder Sensitivity in Cyclosporine A-Treated Rats,” Human and Experimental Toxicology pii: 0960327116660864, available Online at Sage Open Medical Case Reports https://doi.org/10.1177/0960327116660864; Ngobili, T. A. and Daniele, M. A. 2016. “Nanoparticles and Direct Immunosuppression,” Experimental Biology and Medicine (New Jersey) 241(10): 1064-1073; Ateyya, H. 2015. “Amelioration of Cyclosporine Induced Nephrotoxicity by Dipeptidyl Peptidase Inhibitor Vildagliptin,” International Immunopharmacology 28(1): 571-577; Travis, J. 2015. “Making the Cut: CRISPR [clustered regularly interspaced short palindromic repeats] Genome- editing Technology Shows Its Power,” Science [American Association for the Advancement of Science] 350(6267): 1456-1457; Abrahimi, P., Chang, W. G., Kluger, M. S., Qyang, Y., Tellides, G., Saltzman, W. M., and Pober, J. S. 2015. “Efficient Gene Disruption in Cultured Primary Human Endothelial Cells by CRISPR [clustered regularly interspaced short palindromic repeats]/Cas [CRISPR-associated nuclease]9,” Circulation Research117(2): 121-128; Ghane Shahrbaf, F. and Assadi. F. 2015. “Drug-induced Renal Disorders,” Journal of Renal Injury Prevention 4(3): 57-60; El-Gowelli, H. M., Helmy, M. W., Ali, R. M., and El-Mas, M. M. 2014. “Celecoxib Offsets the Negative Renal Influences of Cyclosporine via Modulation of the TGF-β1/IL-2/COX-2/[transforming growth factor β1/interleukin-2/cyclooxygenase-2] Endothelin ET(B) Receptor Cascade,” Toxicology and Applied Pharmacology 275(2): 88-95; Pazhayattil, G. S. and Shirali, A. C. 2014. “Drug-induced Impairment of Renal Function,” International Journal of Nephrology and Renovascular Disease 7:457-468). It warrants emphasis that results obtained with common laboratory animals such as murids, lagomorphs, felids, canids, or porcines may not translate into an effective treatment for humans.

A ductus side-entry jacket positioned about the blood supply of the allogeneic transplant kidney with which it is harvested to target immunosuppressants, to include the targeted delivery of checkpoint inhibitors or blockers posing no significant risk of adverse side effects, and antimicrobials to the transplant organ with another placed about a larger vessel into which an infusion catheter would conventionally be placed to deliver multipotent hemopoietic, or hematopoietic, stem cells to induce chimerism is likely to increase the odds for acceptance (see, for example, Bigenzahn, S., Pree, I., Klaus, C., Pilat, N., Mahr. B., and 4 others 2016. “Minor Antigen Disparities Impede Induction of Long Lasting Chimerism and Tolerance through Bone Marrow Transplantation with Costimulation Blockade,” Journal of Immunology Research 2016:8635721; Granados, J. M., Benichou, G., Kawai, T. 2015. “Hematopoietic Stem Cell Infusion/Transplantation for Induction of Allograft Tolerance,” Current Opinion in Organ Transplantation 20(1): 49-56; Cetrulo, C. L. Jr., Drijkoningen. T., and Sachs, D. H. 2015. “Tolerance Induction via Mixed Chimerism in Vascularized Composite Allotransplantation: Is It Time for Clinical Application?,” Current Opinion in Organ Transplantation 20(6): 602-607; Hock, K., Mahr, B., Schwarz, C., and Wekerle, T. 2015, Op cit. above under Antepartum Metered Switch Heart Transplantation; Kim, N., Lee, H., Shin, J., Nam, Y. S., Im, K. I., Lim, J. Y., and 4 others 2015. “Immune Reconstitution Kinetics following Intentionally Induced Mixed Chimerism by Nonmyeloablative Transplantation,” Online, PLoS [Public Library of Science] One 10(5): e0126318; Mathes, D. W., Chang, J., Hwang. B., Graves, S. S., Storer, B. E., Butts-Miwongtum, T., Sale. G. E., and Storb. R. 2014. “Simultaneous Transplantation of Hematopoietic Stem Cells and a Vascularized Composite Allograft Leads to Tolerance,” Transplantation 98(2): 131-138; Burlingham, W. J. 2014. “Exosomes: The Missing Link Between Microchimerism and Acquired Tolerance?,” Chimerism 5(3-4): 63-67; Wang, M., Racine, J., Zhang, M., Wu. T., Deng. R.5., and 4 others 2014. “MHC [major histocompatibility complex]-mismatched Chimerism is Required for Induction of Transplantation Tolerance in Autoimmune Nonobese Diabetic Recipients,” Journal of Immunology (Baltimore, Maryland) 193(4): 2005-2015; Leventhal, J., Abecassis, M., Miller, J., Gallon, L., Tollerud, D., and 5 others 2013. “Tolerance Induction in HLA [human leukocyte antigen] Disparate Living Donor Kidney Transplantation by Donor Stem Cell Infusion: Durable Chimerism Predicts Outcome,” Transplantation 95(1): 169-176; Graves, S. S., Mathes, D. W., Georges, G. E., Kuhr, C. S., Chang, J., Butts, T. M., and Storb, R. 2012. “Long-term Tolerance to Kidney Allografts after Induced Rejection of Donor Hematopoietic Chimerism in a Preclinical Canine Model,” Transplantation 94(6): 562-568).

Infusion of both the directly targeted immunosuppressants and antimicrobials and the stem cells directly into the systemic circulation is through different openings in the same body surface port ordinarily positioned in the pectoral region, each opening connected to its respective side-entry jacket, or jackets, connector, or connectors. The complete sealing off the surface port to the surrounding environment and of the side-entry jackets and connectors to the internal environment, and the fact that these devices are designed to allow the direct insertion of antimicrobials and other drugs such as immuno-suppressives, anti-inflammatoires, and anesthetics make side-entry less infection prone than conventional devices.

Moreover, so long as the surface port remains in place, the targeted administration of stem cells and drugs can be repeated without the need to repeatedly subject the patient to venipuncture in order to inject drugs not targeted directly to the transplant and a needles and line to accomplish stem cell delivery. Long-term dialysis or apheresis through an extracorporeal dialyzer or apheresis machine is through a body surface port with two openings, one the blood inlet and the other, the blood outlet. If necessary, other openings and lines are connected to side-entry connectors and/or jackets to diagnose, view, or treat other sites in disseminated and/or comorbid disease.

Rather than connected to a small caliber peripheral vein, the port is connected to a major vessel, such as the abdominal aorta or inferior vena cava, through two adjustable side-entry diversion jackets, that superior (cephalad, craniad, higher) usually inverted when the circulation is clockwise facing the patient and that inferior (caudad, lower) uninverted. The diversion jacket accessory channel or channels can deliver drugs and other agents to, and incorporate sensory probes within, the jacket at the treatment site and into the continuing lumen of the ductus as needed.

The creation of catheteric access is subject to complications such as a perforation and troublesome bleeding from the carotid or femoral artery, the jugular, subclavian, or femoral vein, for example, or penetration into the lung, and albeit rarely, infection (see, for example, Ge, B. H., Copelan, A., Scola, D., and Watts, M. M. 2015. “Iatrogenic Percutaneous Vascular Injuries: Clinical Presentation, Imaging, and Management,” Seminars in Interventional Radiology 32(2): 108-122). Because this preparation for connection to the machine must be accomplished in the clinic, takes about a half hour, with the ensuing dialysis lasting about five hours, rather than to remove the line, the line is left in place and if discharged, the patient asked to return for treatment five or seven days a week.

The patient who is competent or has a trained assistant is therefore prepared for initial discharge to continue treatment at home using a different method requiring less than professional skill until an alternative means of access requiring a second procedure can be performed and allowed sufficient time to heal. Most often, the second procedure will be the creation of an arteriovenous fistula. Once the anesthetic wears off, the surgical wound usually causes discomfort. If severe, repeated injections of an anesthetic such as lidocaine are required.

However, since a side-entry diversion jacket is placed with a body surface port and means for automatic drug delivery, providing pain relief following placement does not require injections. Furthermore, the establishment of immediate access using the means described herein will remain that to continue in use without the need for frequent venipuncture until a transplant is found. The result is increased patient satisfaction in the clinic, with optimization in machine time, and increased patient throughput, especially using a high flux rate dialyzer, and increased patient satisfaction when discharged. Should a transplant be found, the surface port, jackets, and lines are left in place to deliver immunosuppressants and other medication as necessary to support the transplant.

In immunocompromised patients, viruses and fungi can also cause infection of the urinary tract (see, for example, Santiago-Rodriguez, T. M., Ly, M., Bonilla, N., and Pride, D. T. 2015. “The Human Urine Virome in Association with Urinary Tract Infections,” Online, Frontiers in Microbiology 6:14; Paduch, D. A. 2007. “Viral Lower Urinary Tract Infections,” Current Urology Reports 8(4): 324-335). However, the distinct majority of urinary tract infections are caused by bacteria, namely Escherichia coli and Staphylococcus saprophyticus. Patients requiring catheterization are additionally exposed to Klebsiella and Pseudomonas, and the fungal pathogens Candida and Enterococcus, all of which the means described herein allow to be eradicated with directly targeted higher doses of the appropriate drugs than would be introduced into the circulation, and without side effects.

Antibiotic resistant urinary tract bacteria include that most prevalent Escherichia coli sequence type 131 (see, for example, Han, J. H., Garrigan, C., Johnston, B., Nachamkin, I., Clabots, C., and 8 others 2017. “Epidemiology and Characteristics of Escherichia coli Sequence Type 131 (ST131) from Long-term Care Facility Residents Colonized Intestinally with Fluoroquinolone-resistant Escherichia coli,” Diagnostic Microbiology and Infectious Disease 87(3): 275-280; Hirsch, E. B., Zucchi, P. C., Chen, A., Raux, B. R., Kirby, J. E., McCoy. C., and Eliopoulos, G. M. 2016. “Susceptibility of Multidrug-Resistant Gram-Negative Urine Isolates to Oral Antibiotics,” Antimicrobial Agents and Chemotherapy 60(5): 3138-3140; Swami, S. K., Liesinger, J. T., Shah, N., Baddour, L. M., and Banerjee, R. 2012. “Incidence of Antibiotic-resistant Escherichia coli Bacteriuria According to Age and Location of Onset: A Population-based Study from Olmsted County, Minnesota,” Mayo Clinic Proceedings 87(8): 753-759; Johnson, J. R., Johnston, B., Clabots, C., Kuskowski, M. A., and Castanheira, M. 2010. “Escherichia coli Sequence Type ST131 as the Major Cause of Serious multidrug-resistant E. coli Infections in the United States,” Clinical Infectious Diseases 51(3): 286-294).

In tuberculosis complicated by intercurrent amyloidosis, “Prompt treatment of the underlying TB focus can prevent progression to end-stage renal disease,” Merchant, S., Bharati. A., and Merchant, N. 2013. “Tuberculosis of the Genitourinary System-Urinary Tract Tuberculosis: Renal Tuberculosis—Part I,” Indian Journal of Radiology and Imaging 23(1): 46-63, see also, for example, Leung—Theung-Long, S., Coupet, C. A., Gouanvic, M., Schmitt, D., Ray, A., and 15 others 2018. “A Multi-antigenic MVA [vaccinia virus Ankara; modified Ankara virus] Vaccine Increases Efficacy of Combination Chemotherapy against Mycobacterium Tuberculosis,” Online, PLoS [Public Library of Sciencee] One 13(5): e0196815; Figueiredo, A. A., Lucon, A. M., and Srougi, M. 2017. “Urogenital Tuberculosis,” Microbiology Spectrum 5(1); Moosavi, B., Fasih, N., Virmani, V., and Kielar, A. 2016. “Beyond Ureterolithiasis: Gamut of Abnormalities Affecting the Ureter,” Clinical Imaging 40(4): 678-690; Kou. Y., Xu. Y., Zhao, Z., Liu, J., Wu. Y., and 5 others 2017. “Tissue Plasminogen Acivator (tPA) Signal Sequence Enhances Immunogenicity of MVA-based Vaccine against Tuberculosis,” Immunology Letters 190:51-57; Siddiqui, K. F., Amir, M., Khan, N., Rama Krishna, G., Sheikh, J. A., Rajagopal, K., and Agrewala, J. N. 2015. “Prime-boost Vaccination Strategy with Bacillus Calmette-Guerin (BCG) and Liposomized Alpha-crystalline Protein 1 Reinvigorates BCG Potency,” Clinical and Experimental Immunology 181(2): 286-296; Leung—Theung- Long. S., Gouanvic, M., Coupet. C. A., Ray, A., Tupin, E., and 9 others 2015. “A Novel MVA [modified Ankara virus]-based Multiphasic Vaccine for Prevention or Treatment of Tuberculosis Induces Broad and Multifunctional Cell-Mediated Immunity in Mice and Primates,” Online, PLoS [Public Library of Science] One 10(11): e0143552; Prakash, J., Goel, A., Sankhwar, S., and Singh, B. P. 2013. “Extensive Renal and Ureteral Calcification Due to Tuberculosis: Rare Images for an Uncommon Condition,” Online, BMJ [British Medical Journal] Case Reports pii: ber2012008508; Kolibab, K., Yang, A., Derrick, S. C., Waldmann, T. A., Perera, L. P., and Morris, S. L. 2010. “Highly Persistent and Effective Prime/Boost Regimens Against Tuberculosis that Use a Multivalent Modified Vaccine Virus Ankara-based Tuberculosis Vaccine with Interleukin-15 as a Molecular Adjuvant,” Clinical and Vaccine Immunology 17(5): 793-801).

In tuberculosis, continued regular direct delivery of medication by an implanted microcontroller following a prescription-program and receiving diagnostic information from implanted sensors to sites likely to become granulomatous is likely to suppress the development of dormant disease with ensuing recrudescence and relapse of the disease (see, for example, Peddireddy, V., Doddam, S. N., and Ahmed, N. 2017. “Mycobacterial Dormancy Systems and Host Responses in Tuberculosis,” Online, Frontiers in Immunology (Lausanne, Switzerland) 8:84; Coppola, M., van den Eeden, S. J., Wilson, L., Franken, K. L., Ottenhoff, T. H., and Geluk, A. 2015. “Synthetic Long Peptide Derived from Mycobacterium tuberculosis Latency Antigen Rv1733c Protects Against Tuberculosis,” Clinical and Vaccine Immunology 22(9): 1060-1069; Siddiqui, K. F., Amir, M., Khan, N., Rama Krishna, G., Sheikh, J. A., Rajagopal, K., and Agrewala, J. N. 2015, Op cit. in this section; Sahdev, P., Ochyl, L. J., and Moon, J. J. 2014. “Biomaterials for Nanoparticle Vaccine Delivery Systems,” Pharmaceutical Research 31(10): 2563-2582; Singh, S., Saraav, I., and Sharma, S. 2014. “Immunogenic Potential of Latency Associated Antigens Against Mycobacterium Tuberculosis,” Vaccine 32(6): 712-716).

Passage by threading or snaking of finely gauged cabled devices through an accessory channel allows the retrieval of luminal contents and biopsy samples of the urothelium (uroepithelium, urinary epithelium) or endothelium for diagnosis (see, for example, Coppola, M., van Meijgaarden, K. E., Franken, K. L., Commandeur, S., Dolganov, G., and 10 others 2016. “New Genome-Wide Algorithm Identifies Novel in-Vivo Expressed Mycobacterium tuberculosis Antigens Inducing Human T-Cell Responses with Classical and Unconventional Cytokine Profiles,” (Nature Research) Scientific Reports 6:37793). Pulmonary tuberculosis and other infectious diseases of the lungs, resistant (refractory, intractable) to drug delivery by the conventional routes, typically, a pneumonia, can be directly targeted to the lungs in higher than systemic doses by placing ductus side-entry jackets on the bronchial arteries, which supply oxygenated blood and nutrients to the lungs.

An acute, chronic, and xanthogranulomatous bacterial pyelonephritis affecting the ureters can be addressed by placing a nonjacketing side-entry connector at the pelvis and a ductus side-entry, or if requiring diversion, a diversion jacket along the affected ureter or ureters to directly target an antibiotic or mix thereof as appropriate based upon the species or strain of the pathogen or pathogens.

2d. Body Surface Side-Entry Ports

As described in U.S. Pat. No. 11,759,186, entitled Ductus Side-entry Jackets and Prosthetic Disorder Response Systems, and illustrated in FIGS. 27, 28, and 45 therein and below in this section and FIGS. 17A, 26A thru 26C, 27C, body surface ports constitute a type of nonjacketing side-entry connector. For medicinal use, such a small surface port is used as incurrent to inject drugs and other agents either directly into the bloodstream through a connecting ductus side-entry jacket or valve jacket, or to extract blood for examination, while for urological use, the port is used as excurrent to pass urine to an intact part of the lower urinary tract, a prosthetic bladder, or ‘neobladder,’ or a collection bag usually cinched about a leg. Urine outflow imposes two structural requirements on a surface port, of which both differ from those of a pectoral portacath or mediport for the injection of drugs and drawing of blood samples.

The benefit in the assist device over conventional surgical procedures is that made of synthetic materials, it is not susceptible to metaplastic degeneration leading to carcinoma, which is a younger patient is a predominant consideration. First, the port must provide a urine effluent outlet, an opening to the exterior for the attachment of a hose leading to a nozzle for emptying in a receptacle of a collection bag, usually worn on the front or outer side of the ipsilateral thigh. Second, a port to drain off urine is best positioned in the lower abdomen as is, in fact, an ileal conduit with stoma, or in the pelvis, not in the pectoral region. As pectoral placement takes greater advantage of gravity, a surface port for exclusively incurrent use to inject drugs is best positioned subdermally in the pelvic region.

That is, while light and small gauge lines allow the port in the lower urinary tract assist device in FIG. 30 to be positioned to a side of the mons pubis, the drug administration function and the outflow or urine could, in fact, be divided between two ports, one pectoral and, the other to a side of the mons pubis. Alternatively, a surface port with druglines and without urine effluent outlet, can be used apart from or in conjunction with surgical diversion means such as an ileal conduit in an elderly patient, either through an effluent outlet placed farther down the axis, or in conjunction with an internal diversion procedure, such as to provide drugs to an Indiana pouch. A port with cutaneous, or epicutaneous, openings is shown in U.S. Pat. No. 11,759,186, FIGS. 27 and 28, a port with subcutaneous openings here in FIG. 26A, and mixed cutaneous and subdermal openings here in FIG. 26C.

TABLE 3
Table 3. Advantages and disadvantages of epidermal, or cutaneous,
and subdermal, or subcutaneous, port openings.

| PORT OPENINGS | ADVANTAGES | DISADVANTAGES |
|---|---|---|
| CUTANEOUS | Clearly viewable injection points | Skin perforated. |
| | when placed in the mons pubis. | Noticeable outside of mons pubis |
| | Does not require backplate. | |
| SUBCUTANEOUS | Skin unperforated. | Except in mons pubis, palpation |
| | Little noticeable outside mons pubis. | or tattoos needed to locate. |
| | Unseen when placed in mons pubis. | Not external/unseen. |
| | | Requires faceplate when a urine |
| | | outlet is required. |

Table 1 above in section 2a, entitled Background of the Invention, generally shows the various parts required by the different types of urinary diversion system according to the concept delineated herein, and Table 3 shortly below specified the advantages and disadvantages of epidermal, or cutaneous, and subdermal, or subcutaneous, port openings. Adjustability requires a means for advancing and retracting the diversion chute; hence, only the surface ports of adjustable embodiments have chute advancement and retraction push/pull cable control knobs. Only an embodiment suitable for daytime use when the wearer can void while erect does not require a neoureter convergence chamber ejection assist impeller battery. Thus, while examining the port does not allow the observer to distinguish from the lack of control knobs that it is nonadjustable, whether it is temporary or permanent will not be apparent.

Small caliber cabled devices such as a fine fiberscope, laser, or intravascular ultrasound probe, can be passed to the termini of the lines passing from the port entirely to the target tissue, here, the jacketed ureter or ureters, by insertion through the effluent outlet opening, or the accessory channels by piercing the skin and if present, a membrane covering over the subdermal entry opening. Needle insertion sites for subdermally positioned accessory channel inlets are marked with small tattooed dots on the skin or small palpation elevations on the front of the port. The ports to be described additionally differ from a portacath or mediport in that injection is not necessarily vascular for direct entry into the systemic circulation, and from conventional insulin pumps in that injection is not necessarily subcutaneous, hence, concentrated at first to disperse and become systemic over an interval.

Here drugs injected into the surface port are directly targeted to the treatment sites through accessory channels to side-entry jackets, side-entry diversion jackets, or nonjacketing side-entry connectors. Such ports can also be used to pass through cabled devices to the same treatment site or sites. Because conventional ports are positioned in the pectoral region and stomas in the abdominal region, the consolidation of incurrent and excurrent functions into a single port does not arise. For the present purpose, drugs for delivery beneath the level of the port and easily pumped to a superior location are provided with openings in the one port, thus consolidating incurrent and excurrent functions to and from sites in the pelvic region. FIG. 26C shows a surface port 147 with external effluent outlet for the urine outflow line 110 and subdermal accessory channel inlets 107.

To target different ductus through flow-nondiverting, or conventional side-entry jackets without urinary diversion, the port is ordinarily placed subdermally in the pectoral region, presenting no evidence to outward view. With urinary diversion, the port must open to the outside, hence, mount externally, usually on the lower abdomen or pelvic region. For implementing a prosthetic disorder response system entrusted with the treatment of comorbid conditions at different levels along the axis to include urinary diversion to an external collection bag, two ports are used. Ductus higher along the axis are addressed through a pectoral surface port without effluent outlet. A second port with effluent outlet and accessory channel inlets for targets lower along the axis is ordinarily placed in the abdominal or pelvic region, the port inlets in either or both placed subdermally or opening beneath the effluent opening cover.

A prosthetic disorder response system is administered by an implant timing module or microcontroller receiving input from sensors positioned to monitor each condition responds by delivering drugs according to its prescription-program. Such a system when including urinary diversion would ordinarily place the controller with the upper of the two ports. With a patient lacking the ability to control the push/pull cables in an adjustable embodiment, control of the diversion shunt or shunts can either be assigned to the controller and small de motors, or more economically, to a nonadjustable embodiment. The controller implements the prescription-program by using inputs from sensors which have been strategically chosen and positioned to monitor the comorbid conditions and activating the pumps at the outlets of the flat reservoirs in response to the conditions detected.

The flat reservoirs receive the drugs by jet or hypodermic injection through respective openings of either or both a subdermal pectoral port and an abdominal port, the only opening to the outside required of the latter when including urinary diversion to the exterior. When the addition of a second port in the pectoral region is unacceptable so that a port positioned as would a stoma must be used to target sites superior to the port, the injection openings must feed directly into subdermally placed flat reservoirs with pumps to drive the injectant or injectants cephalad (craniad). This is possible but not preferred as discounting the advantage of a pectoral port with gravity feed so that only a release valve and not a pump is controlled, necessitating an increase in componentry, possibly needless complexity and chances for complications, to include a need to reenter.

With respect to replenishment of implanted drug reservoirs as opposed to internal drug delivery from those reservoirs, in FIGS. 26 and 27 of U.S. Pat. No. 11,759,186, entitled Ductus Side-entry Jackets and Prosthetic Disorder Response Systems, shows a body surface (epidermal, cutaneous, epicutaneous, on-skin-mounted) body surface port without urinary diversion opening, or effluent outlet, to be positioned in the pectoral region for targeting drugs and agents for maintaining the apparatus by direct piping into internal reservoirs.

There, external rather than subdermal positioning of the entry openings in a drug inlet port served to admit drugs through tubes attached to pumps housed in an externally worn power and pump pack. However, when drugs are jet or needle injected, that is, pumped through jet injectors or hollow needles rather than through tubes, subdermal (subcutaneous) placement is to be preferred as allowing full, or closed-skin, implantation, which is less susceptible to infection or being struck as well as esthetically preferable as unnoticeable.

Herein, FIG. 26C shows a sectional view through a combination incurrent/excurrent abdominal port 147 shown in context as the body surface portal of the lower urinary tract assist system shown in FIG. 30 for the expulsion of urine and the targeting drugs and maintenance agents such as a crystal solvent to the parts of the apparatus and the ureters. Isolated from sources of infection, the subdermally placed openings are entered transdermally (percutaneously) by multiple discharge jet or hypodermic needle injectors such as those shown in FIGS. 27A and 27B.

In a surface port with the effluent outlet at the surface and the injection or infusion inlet or inlets placed subcutaneously, the frontal mounting plate is the faceplate 231, and the rear backplate is 104. While membrane 121 covered incurrent openings into the druglines 8 are situated subcutaneously, urine effluent outlet pipe 110 passes through faceplate 231 to the body surface where screw or snap shut cover 108 cuts off entry other than when removed during voiding. Shown in FIG. 30, urine effluent outlet pipe 110 can be connected by hose 149 to drain directly into a restroom receptacle or plastic urine collection container or bag 148 usually cinched about a thigh.

Fixation of the port is by sutures through the backplate 104 provided with suture loops (not shown) or perforated for the purpose and through the subcutaneous fat and fascia propria musculus. A hole for the effluent outlet is made through the skin with a 4.0-millimeter skin punch and the baseplate with L-connectors attached placed through a small neighboring incision. Referring to FIGS. 28 and 30, side-entry diversion jacket (in a unilateral embodiment) or as shown, jackets 143 and 143′ with neoureters 144 and 144′ as mainlines empty into neobladder 145 can be placed before or after placement of surface port FIG. 26. Druglines, or sidelines, 8 and 8′ likewise lead entirely from the valves through neoureters 144 and 144′ to the effluent opening to allow the direct targeting of system maintenance agents entirely through the lower urinary tract prosthesis shown in FIG. 28 or the assist device shown in FIG. 30.

The body surface port shown in isolation in FIG. 26C and in its anatomical context in FIG. 30 is preferably thermoset press or compression molded out of thin flat polypropylene or polyetheretherketone sheet stock with frontplate 231 and body surface port backplate 100 perforations in the die. In FIG. 26C, perforated body surface port backplate 100 is fixed in position as indicated by sutures 104 which pass as specified above through the subcutaneous fat and fascia propria musculus. Excess suture is easily snipped off with an orthodontic wire cutter. Body surface port faceplate 231 and backplate 100 perforations assure that the underlying tissue will not be subject to complete enclosure. Both body surface port frontplate 231 and body surface port backplate 100 can incorporate unused perforations to allow for the addition of lines as may become necessary.

In FIG. 26C, urine effluent or outlet pipe 110 has an external thread or equivalent external complement of a snap joint to accept dome-shaped port cover cap 108 with complementary internal thread and has at its base a surrounding flange 232 to rest upon the skin with a nonimitating rounded edge slightly undercut on the inner side to allow the surface of the skin beneath the cover to be wetted with a medicinal emollient or suitable steroid counterimitant, such as 0.1% topical cream triamcinolone acetonide, with the cover removed if necessary. The dome-shaped (inwardly directed) reentrant backside of port cover cap 108 is lined with foam 109. Subcutaneously positioned accessory channels 8 and 8′ are of portacath or mediport form with self-sealing cover membrane open to the outside, and may number more than two as the section view shows.

### Ductus Coupling and Flow Diverters

FIG. 31A shows a tube-within-a-tube ductus coupling and flow diverter 155 for insertion into the cut end of a vessel other than one that supplies or drains the brain or the heart, or into a ureter where some part of all of the lower urinary tract is missing. Such a flow diverter deviates from the paramount object of allowing vascular procedures without the need to interrupt circulation as a significant cause of adverse complications and is meant for use in circumstances where this factor is noncritical. When straight rather than curved, the device can be positioned transluminally.

Diversion in the vascular tree allows bypassing an intervening defect and can be used to shunt a small proportion of blood to a larger vessel in order to reduce a localized hypertension, for example. In the lower urinary tract, diversion into a synthetic bypass ‘neoureter’ for drainage into a synthetic neobladder or directly into a paracorporeal collection bag makes possible a prosthetic lower tract where that native would otherwise have to be reconstructed out of gut. Gut is ill adapted to such use, and its harvesting creates an additional site for potential complications.

The use of synthetic materials allows clear transparency and eliminates the complications associated with tissue, to include degeneration and infection. At the same time, clot in the vascular tree is easily dispelled by the direct release of an anticoagulant drip and crystal in the urinary tract by a crystal solvent. FIG. 31A depicts the diverter as an elbow-shaped tube within a tube 155 where that inner 156 is continuous and that outer 157 is interrupted by pointed retention prongs, or barbs, 158 molded or die cut and pressed outward to project down and outward into the tunica mucosa of a ureter or intima of an artery, at the same time providing an open space or aperture 159 beneath each prong through which drugs released into the space between inner and outer tubes gain access to the surrounding tissue.

Whether molded in plastic or made of metal, outer tube 157 is coated with a polymer that incorporates an anti-inflammatory and antimicrobial. Drugs are delivered into the space between the inner 156 and outer 157 tubes through drugline, or sideline, 8, and drugs and device maintenance agents are delivered into the inner tube 156 through drugline 8′. If die cut and punched metal (usually stainless steel or titanium), retention prongs 158 are bent convex upward, and if plastic (usually polyethylene terephthalate or polyether ether ketone), molded with a convex upper surface, so that medication or device maintenance agents released into the inner tube will flow past rather than through sub-prong apertures 159. FIGS. 31A and 31B show only intermittent prongs; the actual density of the prongs is actually much greater.

As a flow diverter, the diverter is limited in application. Much the same device, usually straight rather than bent and also double-ended, has other applications, such as maintaining certain transplantation anastomoses where immunosuppressives are best applied directly and kept from other tissue, and securing the end of a synthetic line in a ductus of larger caliber. Depending upon the anatomical context and the potential need to pass a transluminal cabled device through a small caliber ductus, a basic side-entry jacket, because it presents no part within the substrate native lumen to interfere with passage of the device, is preferable.

By the same token, whether the device is bent as a urinary flow diverter or straight as an anastomotic coupler or stent, where alternative methods for suppressing the runoff of a residue such as magnetic or the downstream release of a reversal agent involve additional complexity and expense, the coupling diverter allows the direct targeting to the endothelium or urothelium surrounding it of medication with negligible if any continuation of the residue through the bloodstream or urinary tract.

This direct to urothelium or endothelium contact form of drug targeting is no less available when a flow nondiverting straight embodiment is transluminally positioned as a stent or is used to form an anastomotic coupling, such as between donor-to-recipient stumps following transplantation. As a stent, the ductus coupling and flow diverter is critically superior to a conventional drug-eluting stent with a finite limited dose. It can deliver different drugs as commanded by the implant microcontroller or microprocessor in response to sensor inputs upstream along the ductus, for example, where, as shown in FIGS. 26A-C and 27 A-C, the drugs can be quickly replenished.

A semicircular flap with upstream edge conformant to the curvature to either side of inner tube 156 suspended in the center thereof with magnetically susceptible tip upstream, tip can be drawn by small electromagnets to bias flow to either side leading into separate lumina, and a flap fully circular would allow switching between full and partial flow. However, incapable of diverting all flow through either branch, this approach is limited in its ability to apportion flow between two downstream ductus. It therefore is unsuitable for use in bypass, or sudden switched-organ, transplantation, carotid endarterectomy, or the extracardiac correction of a transposition of the great arteries.

The passage of flow through a length of tubing with opposing diverters at either end for use as an insert patch to reconnect the ends of ductus which due to disease had to be excised or injury had been lost. Otherwise, pending sufficiency in the generation of the type ductus by means of tissue engineering, the need to harvest a length of native ductus from the patient, usually a vein, will continue to demand a preliminary invasive procedure and create the risk of serious complications at the site of harvesting.

A semicircular flap with upstream edge conformant to the curvature to either side of inner tube 156 suspended in the center thereof with magnetically susceptible tip upstream tip can be drawn by small electromagnets to bias flow to either side leading into separate lumina, and a flap fully circular would allow switching between full and partial flow. Such measures do make possible the modulation of flow through a length of tubing with opposing diverters at either end for use as a prosthetic insert patch to reconnect the end of a ductus which due to disease or injury had to be excised. Otherwise, pending sufficiency in the generation of the type ductus by means of tissue engineering, the need to harvest a length of native ductus from the patient, usually a vein, will continue to demand a preliminary invasive procedure and create the risk of serious complications at the site of harvesting.

To assure transluminal passage free of any artificial obstruction as could hinder a future need for angioplasty or examination with intravascular ultrasound, coronary and carotid arteries, for example, are best kept from the insertion of any device or part thereof within the lumen, making side-entry jackets superior to a straight double-ended embodiment of the diverter shown in FIGS. 31A and 31B. Retention prongs, or barbs, 158 and apertures 159 are not arranged in vertical lines but rather at radially diverse locations about the circumference and length of outer tube 157. Delivery of a fluid drug into the space between inner tube 156 and outer tube 157 through drugline 8 is beneath the closed off upper ends of double tube 155 under the slight pressure imparted by the implanted drug reservoir outlet pump or drip stopcock.

This causes the drug to flow about and down between tubes 156 and 157 to flow out through the spaces 159 beneath each prong 158 to wet, and depending upon the chemistry and viscosity of the drug, become absorbed into the tunica mucosa of the ureter or endothelium of the vessel. FIG. 31B shows the outflow pattern of a drug released from an implanted reservoir delivered into the space between inner tube 156 and outer tube 157 through the space 159 beneath each retention prong 158.

This pattern serves to deliver an anti-inflammatory, anesthetic, and/or antimicrobial directly into the small wound created by penetration of each retention prong 158 that would otherwise cause discomfort or pain referred to one or both flanks, the back, upper sartorius, pectineus, and/or adductor longus, and/or the testes or labia majora until the body responded by enclosing each prong within a fibrous capsule. Retention prongs 158 are depended upon to counter dislodgement due to downward migration. The palliation of pain should be limited to the direct topical application of an anesthetic, not a crushing of nervelets as could adversely affect peristalsis and/or induce atrophy. Fitted properly, peristalsis will eventually secure the full penetration into the tunica mucosa of the prongs.

For transluminal placement, it is imperative that the retention prongs, or barbs, 158 not scrape against the endothelium or urothelium en route. Various substances can be used to cover over these over before being dissipated once the device has reached its destination. Coating the outside of a ductus coupling to be placed in the manner of an endoluminal stent with a concentrated solution of a sugar solid during placement can if necessary, be followed by the release through the barb apertures 159 of warm distilled water to accelerate its dissolution. A refrigerated slice of butter in a stack thereof separated by wax paper can be wrapped around the device, then placed in a freezer. Upon reaching the destination, accelerated dissolution of this barrier layer is through the release through barb apertures 159 of suitable enzymes (lipase, lactase).

Accordingly, the tube within a tube diverter 155 is inserted into the cut end, or stump, of the ductus so that it fits snugly against the noncompressive descending zones (receiving segments, leading waves of distension, stenosis regions) of a ureter or the systoles along the passing waves of an artery. However, it does not significantly press radially outward as would compress the small vessels whether branches proceeding caudally of the abdominal aorta, common iliac, renal, superior and/or inferior vesical, gonadal (testicular or ovarian), middle rectal and/or uterine arteries.

Closer to the bladder, the superior vesical, middle rectal, in men the inferior vesical, and in women, the uterine and vaginal arteries which provide tiny branches that inter-anastomose just inside the adventitia (fibrosa, fibrous coat) and submucosa; accompanying nervelet branches of the renal, aortic, and/or hypogastric autonomic plexuses; and or lymphatics of the aortocaval and common iliac nodes in the abdominal ureter or the internal and external iliac nodes in the pelvic ureter as would promote atrophy.

Depending upon the state of the urinary tract, diversion can be to the native bladder, constructed (unpreferred) or synthetic neobladder as preferred through a nonjacketing side-entry connector as shown in FIGS. 12A and 12C of U.S. Pat. No. 11,013,858. When possible, rejoinder from prosthetic neobladder 145 to the urethra rather than connection to an outlet hose 110 is preferable as allowing normal voiding. However, in the absence of urge sensation, voluntary control requires a tactile or audible signal, which is easily accomplished, and except in prostatectomized males with favorable anatomy, clear access to the urethra for this purpose is likely to prove elusive.

When feasible, much dissection is involved. Given the limited working space, connection to the urethra is more easily accomplished with a double-ended version of the device shown in FIGS. 31A and 31B rather than a side-entry jacket. Referring to FIGS. 28 thru 30, with the nonvalve diverter as in valves systems, when the lower tract is actually or effectively missing, diversion is to a prosthetic neobladder 145 and out drainage line 110 opening to the exterior at the center of port type 147 and into a collection bag 148 ordinarily cinched about a thigh, or where no neobladder is present, then drainage is directly through line 110 and into the collection bag 148. The positioning of either a side-entry diversion jacket or a diverter along a ureter, for example, is best at the level with the largest internal diameter remaining intact.

The justification for the placement of an implant is that it is necessary or would provide greater benefit than harm. There is no good place to position a foreign object inside the body. Anywhere an implant is placed without adverse reaction protective countermeasures can and will lead to complications. One tactic is to allow the body to overreact to the foreign body by initiating a cascade of cell signaling peptides, or cytokines, often as self-destructive as remedial or protective, but immediately suppress if not terminate the reaction through the directly pipelined delivery to the root, nidus, or site, of disease of suitable medication to include a glucocorticoid and a synthetic immunosuppressive, for example. Drug eluting stents exemplify this approach in a simple form

The choice between an intraluminal and extraluminal device must be a clinical judgment based upon the specific conditions encountered. The devices and methods delineated herein are not necessarily meant to supplant accepted practice but rather provide alternative options to make possible a more appropriate treatment of specific conditions. Inflammation generates detectable analytes which sensors can report to an implant microcontroller for response with the direct pipeline targeting of a palliative, remedial, or reversal agent. Side-entry jackets are positioned in surrounding relation to the substrate ductus leaving the lumen clear of a foreign object but must present radial projection.

By the same token, lined with foam, peristalsis, the pulse, swelling infection or inflammation, or growth is accommodated, and migration prevented, usually without even an occasional tethering to nearby tissue by running suture through a suture eyelet, part number 15 in the accompanying drawing figures. Inside a peristaltic ductus, to minimize stress on the smooth muscle restrained from movement by a solid insert device, the endoluminal diverter or end-to-end stump connector should be kept short. In the devices described here, the adverse sequelae associated with intra- or extraluminal contact between an endoluminal implant such as a diverter with the endothelium or urothelium or between a surrounding jacket and the adventitia or fibrosa have been taken into account and responded to by providing druglines that immediately target medication directly to the site of the irritation.

An endoluminal diverter such as that shown in FIGS. 31A and 31B or an extension coupling, or bridge section with straight, or nondiverted end-to end stump connectors at either end to allow reconnection of the stumps of a ductus that had to be resected or was irreparably damaged is positioned within and does not project outside the substrate ductus, thus eliminating the risk of abrasive contact with neighboring tissue. However, absent the direct delivery of medication to the implant-endothelial or endothelial interface within the ductus, the implant would likely irritate the ductus lining, and if long, unduly interrupt the passage of peristaltic or pulsatile waves and in small gauge lumina, may prohibit or make more difficult passage with an intravascular ultrasound probe or excimer laser, for example.

Entirely inside the ductus, the diverter or end-to-end connector is less accessible to the interventionalist or endoscopist. Where side-entry devices can generally be placed with the patient under local or regional anesthesia, an endoluminally inserted diverter requires transection of the substrate native ductus. With a coronary or carotid, where clamping poses considerable risk, placement of an endoluminal implant usually necessitates cardioplegia, cardiopulmonary bypass, and general anesthesia. Used in the urinary tract, a diverter or end-to-end connector is simpler and less expensive than a side-entry device.

However, at the end of a severed ureter, it is fixed in diverting flow or bridging it to another severed ductus and cannot switch between diverted and nondiverted flow, much less apportion flow between the two. By the same token, in a prosthesis, which must remain fixed, this nonadjustability is advantageous as a safeguard against improper use.

### SECTION 3. SUMMARY OF THE INVENTION

Described is a ductus side-entry jacket with a negative feedback-controlled extendable and retractable lumen-inline flow-diverting chute—that is, a vascular servovalve-having a wide range of applications, to include urinary diversion at the ureter and throughflow controlled shunts or bypasses in the bloodstream. Use to achieve instant switching of the blood supply from the native through a synthetic bypass device allows repair or replacement of the native structure without the need to harvest, tubulate, transplant, and counter physiologically misassign native tissue as a diversion conduit, such as using gut to convey urine. This application expands upon the provisions of U.S. Pat. No. 11,759,186, entitled Ductus Side-entry Jackets and Prosthetic Disorder Response Systems, by describing side-entry jackets adapted to function as vascular servovalves.

Such constitute valves which can be used to redirect all or a controllable portion of the flow through a native ductus through a shunt or bypass into another native ductus. The incorporation into such valves of one or more accessory channels allows the direct targeting of drugs such as anticoagulants, antimicrobials, anti-inflammatoires, anesthetics, anticrystallization reagents, and/or others into the valve and the synthetic line into which the valve diverts the flow. The lumen through the jacket side-stem is first connected to a vacuum pump to remove a plug of tissue of like diameter as the native lumen in the wall of the substrate ductus through which the diversion chute will pass.

The vacuum draws the ductus wall past the trepan leading edge of the side-stem tube to cut the opening for the chute into the substrate ductus. The opposite internal wall of the ductus is protected from gouging injury by a limit switch that stops the suction the moment the plug has been cut. The plug can be extracted through the mainline by the vacuum pump, through a capillary gauge catheter connected to the pump, or a guidewire with a hook at the end. The control of vascular valve diversion chutes can be manual or automated. Manual control pertains to the night-time diversion by the user of urine from either or both ureters to a paracorporeal collection bag by adjustment through Bowden cables controlled at a body surface port, or by the simultaneous switching of plural solenoid-controlled valves during an open procedure.

In transplantation, control is automatic by an implant microcontroller or microprocessor used to coordinate the vascular servovalves connecting like vessels of donor and recipient in response to physiological indicia of the recipient detected by sensors implanted at appropriate locations. Use to switch the blood supply of a recipient from one or more of his own organs to those of a donor allows the gradual transfer of the organ or organs from the donor into his own circulatory system to constitute a new ischemia-reperfusion injury free method of solid organ transplantation. If an intact donor is unavailable, an ex vivo machine-perfused organ can be used.

Heart transplantation thus eliminates the need for general anesthesia, cardioplegia, clamping, and allows avoiding a significant interruption or transient in mean arterial pressure and its potentially serious consequences such as graft organ perfusion-reperfusion injury and cognitive dysfunction. Depending upon the application, the level of control ranges from bistable, meaning fully open or fully closed with a damped push-pull solenoid that moves the diversion chute from fully retracted to fully extended to divert urine, to variable when manual, to precise continuous variability under the control of a microprocessor-governed linear servomotor, for example. Such a jacket allows the diversion of flow from the lower urinary tract if missing or injured indefinitely, precluding any sensation of urgency, while for intractable frequency that interferes with public performance of sleep, the wearer can control diversion as needed.

Inline coupling jackets allow the automatic replacement of a segment in a large vessel, typically the thoracic aorta, under the control of an extracorporeal or a fully implanted master control microprocessor without interruption in the flow of blood, and the replacement segment is expandable to allow growth in an infant without the need for reentry before late adolescence if not for life. Various pressure-adapting negative feedback-controlled shunts and bypasses are described which can be incorporated into otherwise what are otherwise conventional surgical procedures.

In one implementation, the present disclosure provides a perivascular jacket for encircling a native vessel, said perivascular jacket having bonded to it a tubular outlet passageway defining a lumen continuous with that of said native vessel, wherein said tubular outlet passageway extends perpendicularly from said perivascular jacket such that said perivascular jacket can be positioned selectively at any anatomically available level and rotational angle, wherein a valve is disposed within the tubular outlet passageway, said valve including a controllably-driven native lumen intromissive tongue, the controllably-driven native lumen intromissive tongue including an arm extending a controllable distance into a lumen of said native vessel, wherein a distal end of the arm includes an upturned distal edge, thereby serving as a valve to draw away or modulate the flow of a volume through said native vessel into said tubular outlet passageway for discharge out of the perivascular jacket through said tubular outlet passageway. In some implementations, the flow of the volume through the tubular outlet passageway can be modulated between zero and one hundred percent upon actuation of the controllably-driven native lumen intromissive tongue between an extended configuration and a contracted configuration. In this way, vessel side entry can provide an effective means of avoiding transection of the vessel and resulting ischemia and/or reperfusion injuries, among numerous other complications.

In some implementations, extension and retraction of said native lumen intromissive tongue is driven by a servomotor configured to provide continuously variable control of the extension and retraction of said native lumen intromissive tongue.

In some implementations, extension and retraction of said native lumen intromissive tongue is driven by a solenoid.

In some implementations, the distal tip of the controllably-driven native lumen intromissive tongue is distally bifid and bidirectional with both craniad and caudad outlets.

In some implementations, the controllably-driven native lumen intromissive tongue is conformed to accept flow from multiple vessels in parallel adjacency such as those encountered in a neonatal heart transplant. In some implementations, the vessels can be small (e.g., tiny) diameter vessels.

In another implementation, the present disclosure provides a perivascular jacket selectively that is positionable along and about a tubular anatomical structure for delivering drugs, passing cabled devices into, and diverting a measured portion of the luminal contents and biopsy tissue samples a lumen of said tubular anatomical structure, the perivascular jacket including: an outer shell (also referred to herein as a “casing”) including semicylindrical halves joined together along a common edge so as to form a plano-type hinge so that, when opened and placed to encircle the said tubular anatomical structure, the semicylindrical halves grip about said tubular anatomical structure as a collar (for example, a stationary collar); wherein a cushioning layer (also referred to herein as a “lining”) configured to protect small nerves and vessels that enter and depart from an outer surface of said tubular anatomical structure lines an internal surface of each of said semicylindrical halves of the outer shell, wherein perforations pass entirely through said outer shell and said cushioning layer are placed to give access to the lumen of said tubular anatomical structure, wherein an opening is defined in the side of said collar into which a side tube with a trepan front edge can be inserted, wherein said side tube is rotatable around and reciprocable along a longitudinal axis of the side tube thereby facilitating excision of a plug of tissue from a wall of said tubular anatomical structure so as to form a side opening in the tubular anatomical structure such that a lumen of said side tube will be continuous with the lumen of said tubular anatomical structure, and wherein said side tube is fixable in rotational angle and depth of penetration into a side of said tubular anatomical structure, wherein a self-locking screw-down cap with internal expansion bushing that fits onto an external thread at the base of said side tube allows said side tube to be fixed in rotational angle and depth of insertion into said side opening of the tubular anatomical structure; and a projectable and retractable tongue-shaped diversion chute with an upturned distal end mounted within said side tube and configured to slide reciprocally to selectively positional depths into the lumen of the said tubular anatomical structure so that a column of bodily fluid upstream of the diversion chute is diverted out through said side opening and said side tube so as to provide continued flow through a synthetic tube, wherein the perivascular jacket includes a vascular valve.

In some implementations, the vascular valve includes a permanent magnet layer along the internal surface of the semicylindrical halves of said outer shell; wherein said magnet layer is disposed between said outer shell and said cushioning layer, wherein the magnet layer is interrupted to accommodate the opening and closing of said collar and the passing through of said perforations, wherein said magnet layer is magnetized to exert an attractive force centrally toward and perpendicular to a longitudinal axis of said collar, making possible the detention and extraction of magnetically susceptible luminal contents.

In some implementations, the vascular valve includes a plurality of electromagnets disposed between said outer shell and said cushioning layer, wherein the plurality of electromagnets are interrupted to accommodate the opening and closing of said collar and the passing through of said perforations, wherein said plurality of electromagnets are selectively energizable to exert an attractive force eccentrically and collectively energizable to exert an attractive force centrally toward and perpendicular to a longitudinal axis of said collar, making possible the detention and extraction of magnetically susceptible luminal contents.

In some implementations, the vascular valve includes a layer of radiation shielding material in concentric relation to a longitudinal axis of said collar, said radiation shield layer interposed between said outer shell and said cushioning layer, wherein said radiation shield layer is interrupted to accommodate the opening and closing of said collar, said radiation shield layer serving to allow the passage of low to moderate radiation dose rate radionuclides and radioactive isotopes through said collar and preceding components preceding said collar without causing radiation injury to surrounding tissue.

In some implementations, the vascular valve includes a layer of radiation shielding material in concentric relation to a longitudinal axis of said collar, said radiation shield layer situated along the internal surface of said outer shell to surround said magnet layer, wherein said radiation shield layer is interrupted to accommodate the opening and closing of said collar, said radiation shield layer serving to allow the passage of low to moderate radiation dose rate radionuclides and radioactive isotopes through said collar and preceding components preceding said collar without causing radiation injury to surrounding tissue.

In some implementations, the side tube is entered by a catheteric side tube subsidiary to said side tube, said catheteric side tube allowing the directly targeted delivery of fluid drugs, medical solutions, and tubing maintenance solutions into said side tube, collar, and lumen of said tubular anatomical structure. In some implementations, the lumen of the tubular anatomical structure is a treatment site. In some implementations, ureterolith and nephrolith solvents can be delivered when said collar is applied along the urinary tract, and antithrombotic medication can be delivered when said collar is applied along the vascular tree.

In another implementation, the present disclosure provides an automatic homeostasis stabilizer and ambulatory prosthetic disorder response system including a plurality of vascular diversion jackets and pumps supplying fluid medicinals to each respective diversion jacket, wherein the pumps are controlled according to a prescription-program by a microcontroller such that: a plurality of physiological parameter sensors including subordinate nodes as negative feedback loops implanted at different locations in the body, the subordinate nodes being configured in a hierarchical control system to said microcontroller and configured to send outputs to said microcontroller such that the microcontroller is a master node, wherein each of the outputs represent feedback, wherein, when a respective output signals an out-of-range condition to the microcontroller, the microcontroller is configured to respond by causing a respective pump to index to and release a medication prescribed for the respective subordinate node in a dose proportional to the out-of-range output signal received, wherein, as the master node, the microcontroller governs the discharge of the prescription-program, including dispensing the medication through each subordinate node in a coordinated manner as governed by the prescription-program so that dosing among the subordinate nodes is interrelated to attain the highest possible overall homeostasis such that the system is able to treat comorbid conditions affecting different organ systems in a coordinated manner.

In some implementations, the automatic homeostasis stabilizer and ambulatory prosthetic disorder response system is radiation shielded so as to make possible direct administration of radioactive drugs or nonradioactive drugs.

In another implementation, the present disclosure provides a perivascular jacket for encircling a native vessel, said perivascular jacket having bonded to it a tubular outlet passageway defining a lumen continuous with that of said native vessel, wherein said tubular outlet passageway extends perpendicularly from said perivascular jacket such that said perivascular jacket can be positioned selectively at any anatomically available level and rotational angle, wherein a valve is disposed within the tubular outlet passageway, said valve including a disk or cone perpendicular to a direction of flow through said native vessel, thereby serving as a local regulator of flow pressure, the disk or cone including an arm extending a controllable distance into a lumen of said native vessel, wherein a distal end of the arm includes an upturned distal edge, thereby serving as a valve to draw away or modulate the flow of a volume through said native vessel into said tubular outlet passageway for discharge out of the perivascular jacket through said tubular outlet passageway. In some implementations, the flow of the volume through the tubular outlet passageway can be modulated between zero and one hundred percent upon actuation of the controllably-driven native lumen intromissive tongue between an extended configuration and a contracted configuration. In this way, vessel side entry can provide an effective means of avoiding transection of the vessel and resulting ischemia and/or reperfusion injuries, among numerous other complications.

In some implementations, the extension and retraction of said disk or cone is driven by a servomotor configured to provide continuously variable control of the extension and retraction of said disk or cone.

In some implementations, extension and retraction of said disk or cone is driven by a solenoid.

### SECTION 4. OBJECTS OF THE INVENTION

One object of the invention is to so reduce the surgical trauma of each operation specified that patients too debilitated to undergo the conventional operation can be provided with an alternative that will yield a superior outcome.

Another object of the invention is to so reduce the surgical trauma to graft organs and the patient that the pool of available organs is expanded and less robust patients will qualify.

A further object of the invention is to so improve the preoperative condition required to undergo a surgical operation that patients otherwise not qualified will qualify.

A key object of the invention is to provide means for gaining entry into the lumen of any vessel at any level and radial angle relative to its long axis without the need to transect the vessel, thereby avoiding the need to interrupt the flow of blood through it during a surgical procedure which conventionally involves a period of ischemia and necessitates the use of cardiopulmonary bypass life support or extracorporeal membrane oxygenation, cardioplegia, and general anesthesia, each of which may cause serious peri- and postoperative complications.

Another object of the invention is to revolutionize solid organ transplantation to the extent that an infant bom with an unsurvivably malformed heart, for example, will incur no decrement in bodily development or cognitive function, or suffer rejection of the organ due to anoxia, for example, or have to confront constant fearful distraction for further risky operations which can result in pain for years but instead be capable of excelling in school and even of achieving eminence.

Another object of the invention is to provide means for the replacement of organs, to include the heart, and major vasculature so congenitally malformed or defective as to preclude normal development if not pose the risk for early death and to do so without neurologically injurious life support, general anesthesia, cardioplegia, or circulatory arrest, and when genetically justified, to do so as early in utero as possible, thereby eliminating the adversity of outcomes obtained with conventional allogeneic tissue repair however flawless.

Yet another object of the invention is to eliminate not only the need for cardiopulmonary support, the interruption in normal blood flow, cardioplegia, and general anesthesia, when pertinent, but the additional procedural time required to induce hypothermia and rewarm the patient, and anastomosis-related vessel transection or intracorporeal stitching subject to leak or dehiscence in, for example, solid organ and endocrine gland transplantation, extracardiac correction of transpositions of the great arteries, and the stitch failure and risk of migration-free repair of aortic arch aneurysms, interruptions, and coarctations, using simpler and far less traumatizing methods while at the same time providing superior outcomes.

A significant object of the invention is to allow the accomplishment of surgical procedures by locally accessible general surgeons which had previously demanded the exclusive and highly technical training of specialists found only at large centers.

Another object of the invention is to make possible orthotopic and heterotopic solid organ transplantation without the need to perform reconstructive measures within the graft organ, impairing its robustness and reducing the odds for a successful outcome.

An object of the invention is to provide a means for connecting a catheter to a blood vessel from a subdermally (subcutaneously) or body surface (epicutaneously, on the skin) positioned port, which for all but short-term use in—a medical center, is superior to a central line or central venous catheter, such that controllable in outlet cross-sectional area from outside the body and securely fastened, it can continue in use indefinitely.

Another object of the invention is to provide a means for connecting a catheter to a vessel from a port positioned subdermally or at the surface of the skin, which avoids the many complications associated with conventional central lines and surface ports, and is versatile, in that it can be used unidirectionally to deliver drugs, fluids, or parenteral nutrition over a wide range of flow rates, can pass cabled devices such as a fiberscope, laser, or intravascular ultrasound probe, and/or using a double lumen line can be used bidirectionally to shunt blood to and from an extracorporeal hemodialysis or apheresis machine, for example, or to bypass urine around an intervening section of the urinary tract or shunt it to a paracorporeal collection bag, and can incorporate physiological, such as venous pressure and other hemodynamic monitoring and metabolic sensors.

An object of the invention is to provide a means for connecting a central line, used for an in a number of purposes, to a vessel through a small incision, hence, without the need for a guidewire and suspension of the line within the vessel, thus eliminating a multitude of adverse complications, recommending the replacement of central lines for extraclinical ambulatory use.

Another object of the invention is to provide tubular anatomical structure, or ductus, flow diversion side-entry jackets suitable for permanent implantation through 1. Avoiding compression of the fine vessels and nerves entering and exiting the substrate adventitia, 2. Achieving access without entry into the luminal stream with the least trauma through the smallest possible puncture wound, 3. Avoiding any presence of the line or catheter or any part of the side-entry jacket within the native lumen, and 4. Providing sufficient openings through the jacket to maximize exposure of the adventitia to the internal environment.

Yet another object of the invention is to provide ductus flow diversion side-entry jacket and body surface port sets which make it virtually impossible for the jacket or body surface port to migrate.

An object of the invention when used in the urinary tract is the diversion of urine at any level along one or both ureters to an external collection bag, thus bypassing an impaired or missing lower tract, urge sensation, and/or the need for mental competency as would allow use of the facilities provided therefor.

An object of the invention when configured as a prosthesis for continuous diversion is to create a permanent path for the release of urine where patient competence to void in a normal manner of pathology, such as neurogenic, trauma, resection of malignancy, or birth deformity had left part or all of the distal tract missing or dysfunctional.

Another object of the invention when configured to allow the temporary diversion of urine by takeoff at the ureters is to expedite healing of the lower urinary when diseased or operated upon, the invention making possible the direct delivery of drugs, to include checkpoint inhibitors or blockers, into the distal tract as well as crystallization preventive agents through the diversion line.

Yet another object of the invention when configured to allow temporary diversion is to enable the patient to divert urine away from the distal tract on a voluntary basis to allow uninterrupted sleep or public performance, and allow normal urge sensation to be suspended and voiding deferred when bathroom facilities are unavailable.

Another object of the invention is enable the diversion of urine through components made of synthetic materials devised to least provoke an adverse tissue reaction and not susceptible to the multiple causes of degradation and medical complications that affect living tissue, especially when harvested from and thus causing injury to a different organ system in a preliminary procedure itself susceptible to complications, the side-entry diversion jackets used readily fitted with sensors and other electronic components to allow diagnostic monitoring and as necessary, the automatic release of drugs to effect therapy and agents to maintain the patency of both the innate and synthetic lumina.

Another central object of the invention is to provide a urinary or vascular diversion coupling valve-jacket with one or more collateral or accessory channels to allows fine scope access for viewing and the direct targeting to the line, jacket, and native lumen of thrombus, crystallization, inflammation, and biofilm counteractants, thereby making possible the use smaller caliber synthetic tubing, or conventional catheters, whether solid, flexible, or spun (woven), to redirect the flow of content through a native conduit without becoming clogged on a permanent basis.

Another object of the invention is to eliminate the need for an ileal conduit and stoma constructed of harvested gut, which absorptive is unsuited to the conduct urine and susceptible to infection, metaplastic degradation, and other disease.

Yet another object of the invention is to provide flow-diverting side-entry jackets, which made of synthetic materials, are readily fitted with and ideally serve as a platform for electronic sensors, imaging probes, a laser, ultrasound probes, as well as therapeutic means such as a warming element, alone or in combination.

Another object of the invention is to provide stream or flow-diverting side-entry jackets, which made of synthetic materials, are readily modified to incorporate permanent of electrical magnets magnetized normal (perpendicularly) to the axis of flow through the encircled ductus, thereby allowing magnetized contents in the passing flow, such as superparamagnetic iron oxide nanoparticulate drugs, to be detained against or drawn into the wall of the native lumen.

An object of the invention in a bilateral embodiment, wherein each ureter is provided with an independent adjustable takeoff and body surface port, is to allow the obtaining of urinalysis test samples from either kidney.

An additional object of the invention when used to divert flow through a vessel is to provide a configuration that eliminates any part of the jacket except the gradually curved upper surface of the chute or any bare metal surface in or alongside the stream, thus considerably reducing if not eliminating the inducement of clot due to turbulent flow and clot-inducing contact, and therewith, the initiation of a cytokine cascade capable of stimulating the formation of clot anywhere in the circulatory system, the more so in a patient already thrombophilic (hypercoagulable, prothrombotic), or prone to clot due to cancer and/or other disease.

An additional object of the invention used to divert flow through a blood vessel is to allow the direct targeting into the distal vessel inferior to the chute of drugs to treat the distal vessel.

Another object of the invention is to provide a much improved connection to a vessel from a port safely and securely fastened at the body surface for use as a central line, such as a central venous catheter.

Yet another object of the invention is to allow the shunting of blood to a hemodialysis or apheresis machine by plugging the machine lines into a small, safe, and secure subdermal port with no line ends exposed making these more susceptible to infection when disconnected, thus eliminating the need to repeatedly accomplish connection requiring much more time to a vessel using a more disturbing method of causing the patient to present a more disturbing appearance.

A governing object of the invention is to provide new methods for solid organ transplantation to significantly increase the pool of available organs.

An object of the invention is to make possible emergency sudden or metered immune tolerance inductive switching of the circulation of a recipient from his own defective solid organ to the healthy organ of a deceased donor on life support, and in so doing, eliminating the cytokine storms associate with death, excision, metabolic and endocrine isolation of the graft organ, cross-clamping, cardioplegia, ischemia, cold storage, and reperfusion injury, for example, lessening the odds for rejection and the late development of vasculopathy, and in so doing, improve considerably the odds for success following a heart of other solid organ transplant.

Another object of the invention is to make possible a new technique for heart transplantation which eliminates the severe surgical and physiological trauma inflicted on both the graft and native organs which severely reduce the prospect for a successful outcome from the very start, to include extensive dissection of both native and graft organs, integrated stress response, cytokine storm, cardioplegia-induced cardiac arrest, cross-clamping, cardiopulmonary bypass, and general anesthesia.

An object of the invention in organ bypass, or sudden switched-organ transplantation is to provide a means for avoiding the need for cardiopulmonary support with cardioplegia, aortic cross-clamping, and the need for general anesthesia during vascular surgery, to include open carotid endarterectomy and heart transplantation, where conventional techniques interrupt the flow of blood, often results in cognitive dysfunction, and sometimes, cerebral stroke, myocardial infarction, and reperfusion injury.

Another object of the invention is to make possible the replacement of a severely myopathic failing heart with two less than ideal replacement hearts, thus expanding the zone of hearts acceptable for transplantation while placing each heart in support of the other.

An object of the invention is to make possible double heart metered heart transplantation, which allows adjustment in the ejection fraction of both hearts working together, as an alternative to the use of a ventricular assist device, for example, when the complications, availability, and/or expense of such a device, particularly in poor and undeveloped parts of the world preclude their use.

Yet another object of the invention is to make possible vascular bypass connections for carotid endarterectomies and procedures that otherwise require entry into the heart, such as correction of transpositions of the great vessels and heart transplantation, which eliminates ischemic time, the need to clamp, transect and anastomose vessels, and therewith, the need for cardioplegia, artificial cardiopulmonary support, chilling, reperfusion injury, and the adverse sequelae these produce.

Another object of the invention is to eliminate the causes for transplant failure other than immune rejection, such as ischemia-reperfusion injury.

Another object of the invention is to provide a means for accomplishing carotid endarterectomies, the correction of transposition of the great vessels, and solid organ, to include heart transplantation, where said bypasses need not be followed by anastomosing the vessels as repaired or redirected but can remain in place to the end of life.

An object of the invention is to provide a means for correcting an uncomplicated transposition of the great arteries without the need to enter the heart.

Yet another object of the invention is to provide a means for adjusting the blood pressure locally or regionally to protect lesions along the pertinent segment of a vessel or impaired tissue without effect on the systemic circulation, the means additionally capable of delivering medication and cabled devices to the treatment site, where the medication can consist of nonradioactive drugs or other agents, subintimally implanted magnetically susceptible grains or bands, and the cabled devices can be fiberscopes, lasers, intravascular ultrasound probes and physiological parameter sensors.

Another object of the invention is to provide a means for directly targeting drugs into ductus to include vessels, the urinary tract, and the ductus of glands in response to an adaptive prescription-program executed by an implanted microcontroller using negative feedback based upon sensors of physiological parameters to maintain homeostasis as an adaptive ambulatory prosthetic disorder response system to instantly apply remedial measures responsive to morbidity and comorbidity.

An additional object of the invention is to provide a standardized major vessel replacement prosthesis to include the aortic arch with supraaortic branches, abdominal aorta, and pulmonary vein, for example, made of a special fabric, which capable of diametrical and longitudinal expansion, allows use in a neonate to accommodate growth until adulthood without the need for revision despite a five-fold or so increase in dimensions.

Yet another object is to provide the fabric specified in the preceding object for use in all prosthetic conduits to include bloodlines, druglines, tie-lines, and confluence conduits, to spontaneously adjust in size for the patient and thereafter expand in step with growth until adulthood without the need for revision despite a five-fold or so increase in dimensions and despite the need to incorporate vaults in alteration with the flats in the expansion structure, which thrombophilic, is made both of a material and a surface treatment to repel clot.

### SECTION 5. DESCRIPTION OF THE DRAWING FIGURES

FIG. 1 shows a valve nonadjustable flow-diverting basic ductus side-entry jacket precursory to a valve as described in U.S. Pat. No. 11,759,186, entitled Ductus Side-entry Jackets and Prosthetic Disorder Response Systems, positioned along the substrate ductus prior to insertion of the trepan tube through the wall of the lumen.

FIG. 2 shows a simple urinary flow diversion jacket adjustable by the operator during placement but not afterwards by the patient, such a diversion-irreversible, or nominally fixed embodiment serving as the ureteral takeoff in a prosthesis such as that shown in FIG. 28 for a patient without a functioning lower urinary tract.

FIG. 3 is a cross-section through side-stem 19 taken along line A-A′ of the diversion jacket shown in FIG. 2.

FIG. 4 shows a forked vessel segment-straddling spring-return double clamp, or straddle-clamp, for briefly stopping flow through a small segment along a blood vessel shown clamped to stop the flow of blood with the segment to be jacketed trapped with blood, thereby facilitating placement of a side-entry jacket, choke valve, or vascular valve without the difficulty of extracting the tissue plug for insertion of the trepan tube when, for example, obstructed due to the presence of a diversion chute in the trepan tube

FIG. 5 shows a urinary diversion valve, manually adjusted by turning a control knob on a small body surface port, where the control knob is connected to a push pull control cable positioned to drive the diversion chute forward or backward.

FIG. 6 provides a cross-sectional view along line B-B′ through the diversion valve shown in FIG. 5.

FIG. 7 is a longitudinal midline sectional view through a flow diversion jacket only capable of fully retracted or extended deployment of the diversion chute, for use along the vascular tree, where a plunger solenoid makes it possible for the operator to deploy multiple diversion chutes simultaneously as is required, for example, in a sudden switch heart transplant, shown with the plunger solenoid positioned to have pushed the flow diversion chute to full extension for full flow diversion.

FIG. 8 shows a flow diversion jacket of the kind shown in FIG. 7, except that the plunger solenoid is reversed in positioned to pull, rather than push, the diversion valve, this configuration placing the solenoid beneath rather than in line with the accessory channel line used to advance the diversion chute, allowing the jacket to be made shorter, shown with flow diversion chute fully extended for full flow diversion.

FIG. 9 shows a pulse tubular linear servomotor for use in a servovalve having an internal arrangement and outer shape suitable for miniaturization to allow use in a vascular servovalve.

FIG. 10A shows a longitudinal midsection through a vascular servovalve driven by a pulse tubular linear servomotor with a form suitable for miniaturization to allow use in a vascular servovalve of the kind shown in FIG. 9.

FIG. 10B provides a detailed view of the mechanism shown in the lower part of FIG. 10A which allows the use of one control knob to both separately engage and move the direct manual fine adjustment device as required to perform a carotid endarterectomy bypass, for example, while engaged during use.

FIG. 10C is a longitudinal section view of the mechanism shown in FIG. 10B.

FIG. 10D is a face-on transectional view of the mechanism shown in FIG. 10C taken along line in FIG. 10D providing a more detailed cross-sectional view of the mechanism shown in FIGS. 10B and 10C.

FIG. 10E provides a detailed view of the mechanism shown in the lower part of FIG. 10A which allows the use of one control knob to both separately engage and move the direct manual fine adjustment device as required to perform a carotid endarterectomy bypass, for example, when released following use.

FIG. 10F is a vertical bisectional view through a simple (fixed, nonvalve, nonadjustable, noncontrollable) single channel, or one-way side-entry flow diversion jacket or flow diversion guide for positioning along the length of a ductus to tap off the flow showing the through-and-through adventitia to endothelial perforations of openings to protect the vasa vasorum and vasa nervorum for use to direct the outlet flow through a bloodline where that inlet had been controlled by a vascular servovalve.

FIG. 10G is a vertical bisectional view through a simple (fixed, nonvalve, nonadjustable, noncontrollable) double channel, or two-way side-entry flow diversion jacket or flow diversion guide for positioning along the length of a ductus to tap off the flow showing the through-and-through adventitia to endothelial perforations or openings to protect the vasa vasorum and vasa nervorum used to redirect streams of columns of blood entering it on either side into a common channel and exiting these to either side, the primary application thereof being to allow the local of systemic circulatory arrest-free, hence, ischemia-free transfer of a solid organ, in particular, an allograft heart, from a first to a second recipient using the circuit shown in FIG. 15B.

FIG. 10H is a vertical bisectional view through a simple (fixed, nonvalve, nonadjustable, noncontrollable) triple channel, or three-way side-entry flow diversion jacket or flow diversion guide for positioning along the length of a ductus to tap off the flow showing the through-and-through adventitia to endothelial perforations or openings to protect the vasa vasorum and vasa nervorum used to redirect streams of columns of blood entering it from below as well as to either side into a common channel and exiting these to either side and above.

FIG. 11 shows the use of diversion valves to exchange the continuation of flow between two vessels as in an extracardiac transposition of the great arteries within one patient or between corresponding vessels in two patients.

FIG. 12 shows the use of bidirectional side-entry diversion servovalves to take the place of separate valves where, as in neonates and infants, space is at a premium.

FIG. 13A shows paired bidirectional diversion jackets without duplication along the same ductus at a nearby level.

FIG. 13B shows duplicate paired bidirectional diversion jackets to allow flow to periodically be switched between the upper and lower pairs when, for example, permanent placement along the forcefully pulsatile great arteries to correct a transposition where an extracardiac repair is practicable but a levering movement of the valve, even though stabilized through use of the suture loops 15, could cause irritation.

FIG. 14A provides a longitudinal section through a servochoke, or vascular choke-driven servovalve.

FIG. 14B depicts a method using a choke-driven servovalve, or servochoke such as that shown in longitudinal section in FIG. 14A, to constrict the blood supply, here the renal artery, inducing hypoxia within the malignancy-unaffected tissue of the renal parenchyma to reduce its radiosensitivity while delivering oxygenating, hence radiosensitizing agents into the carcinoma of carcinosarcoma (sarcocarcinoma) through a valve jacket feeding a radioactive drip where the level of radiation, or dose rate, is small enough that the material of the catheter and other components provides sufficient shielding.

FIG. 14C shows the radioactive drip-delivering components clad with a temporary radiation shield for the delivery of higher dose rate radionuclides, for example, than the polymeric material of an unshielded line would allow, while FIG. 14C provides a longitudinal section through a servochoke, or vascular choke-type servovalve, shown as part number 166 in FIGS. 14A and 14B.

FIG. 15A is a simplified schematic representation of the pattern of blood flow at the half-way point in a metered switched-organ, or compound vascular bypass, orthotopic heart transplant, or at the end-point in a heterotopic double heart transplant at which time the positions of the vascular valve diversion chutes are positioned at the midway point of extension during reciprocal cross-circulation. Shown with a donor and recipient having nonanomalous vasculature, the arterial supply summarized at the top with the venous return summarized at the bottom.

FIG. 15B shows the use of double outlet side-entry flow diversion jackets as shown in FIG. 10G to produce the circuit used to recover an allograft heart from a first recipient whose native heart has recovered for placement in another patient.

FIG. 15C shows a grouped vessel inlet-outlet confluence conduit, or prenate/neonate confluence conduit with native vessel-bloodline inlet and bloodline-native vessel outlet valves that allow the input and output vessel to share common diversion valves.

FIG. 15D provides a detailed view of the grouping or gathering device positioned as indicated in FIG. 15C.

FIG. 16 elaborates upon the simpler schematic representation of FIG. 15A in showing that in an orthotopic switch, or compound vascular bypass, heart transplant, flow through the graft organ is normally channeled from each vessel of the recipient heart directly to each of the corresponding donor arteries and veins, the small arrows indicating the direction of blood flow.

FIG. 17A shows the preferred positions of the added heart at the completion of a switch double heart heterotopic transplant to the right-hand side of that native and as most directly represented in FIGS. 15A and 16, flow between only the native and added aortae, lower venae cavae, and lower right pulmonary veins shown for visual clarity.

FIG. 17B shows a schematic artery and vein representation of a direct aortocavally connected graft organ-usually a switched-organ heterotopically transplanted second heart or a kidney, when positioned in the lower abdomen where in the vessel have been shown as unitary, or summarized, for clarity.

FIG. 18A shows the action of the arterial blood flow diversion chutes in a sudden switch organ transplant wherein each solenoid driver moves its arterial flow diversion chute from the fully retracted chute position shown in FIG. 18A to the fully extended position shown in FIG. 18B whereby arterial blood flow is bypassed from the native heart on the right and through the donor organ on the left.

FIG. 18B shows the action of the arterial blood flow diversion chutes in a sudden switch organ transplant wherein each solenoid driver moves its arterial flow diversion chute from the fully retracted chute position shown in FIG. 18A to the fully extended position shown in FIG. 18B whereby arterial blood flow is bypassed from the native heart on the right and through the donor organ on the left, at which point the graft organ has been transferred into the circulatory system of the recipient.

FIG. 19A shows the action of the venous blood flow diversion chutes in a sudden switch organ transplant wherein each solenoid driver moves its venous flow diversion chute between the fully retracted chute position shown in FIG. 19A whereby venous flow is through the native organ, to the fully chute extended position shown in FIG. 19B wherewith venous blood flow is bypassed from the native and through the donor organ.

FIG. 19B shows the action of the venous blood flow diversion chutes in a sudden switch organ transplant wherein each solenoid driver has moved its flow diversion chute between the fully chute retracted chute position shown in FIG. 19A whereby flow is through the native organ, to the fully extended position shown in FIG. 19B wherewith venous blood flow is bypassed from the native and through the donor organ, at which point the graft organ has been transferred into the circulatory system of the recipient.

FIG. 20A shows the action of the arterial blood flow diversion chutes in a metered switch organ transplant wherein each servomotor moves its respective flow diversion chute between the fully retracted position in FIG. 20A, through the intermediate position in FIG. 20B, to the fully extended position in FIG. 20C at which point the graft organ has been transferred into the circulatory system of the recipient.

FIG. 20B shows the arterial blood flow diversion chutes in a metered switch organ transplant at the intermediate position where reciprocal cross-circulation exchanges the maximum volume of donor and recipient blood between the two.

FIG. 20C to the fully extended position in FIG. 20C at which point the graft organ has been transferred into the circulatory system of the recipient.

FIG. 21A shows the action of the venous blood flow diversion chutes in a metered switch organ transplant wherein each servomotor moves its flow diversion chute between the fully retracted position shown in FIG. 21A, through the intermediate position shown in FIG. 21B, to the fully extended position shown in FIG. 21C.

FIG. 21B shows the arterial blood flow diversion chutes in a metered switch organ transplant at the intermediate position where reciprocal cross-circulation exchanges the maximum volume of donor and recipient blood between the two.

FIG. 21C to the fully extended position in FIG. 20C at which point the graft organ has been transferred into the circulatory system of the recipient.

FIG. 22A shows the disposition of the jackets and bloodlines before the left lung as exemplary has been harvested from the donor and after it has been placed in the recipient in FIG. 22B.

FIG. 22B shows the disposition of the jackets and bloodlines after the left lung as exemplary has been placed in the recipient.

FIG. 22C shows an anastomosis-supportive bypass.

FIG. 22D shows an anastomoses-supportive bypass with the interpositioning of a length of synthetic tubing secured at either end by means of end-inserted connectors such as that shown in FIGS. 31A thru 31C.

FIG. 22E shows an anastomoses-supportive bypass with the interpositioning of a segment of autologous vessel, gut, or nerve.

FIG. 22F shows a tuned, or negative feedback responsive vascular servovalve-controlled, shunt for placement in the abdominal cavity to relieve the excess pressure of portal hypertension, for example, by tapping off a volume of blood for transfer from the hepatic portal vein into the inferior vena cava, the equivalent arrangement used in the chest to relieve pulmonary hypertension by tapping off a volume of blood for transfer from the pulmonary trunk to the descending aorta.

FIG. 23A provides an overall perspectival view of a patient with a surgical chest dome that imparts the ability to breathe spontaneously during chest surgery without the inducement of a pneumothorax.

FIG. 23B provides an overall side view of a patient with a surgical chest dome that imparts the ability to breathe spontaneously during chest surgery without the inducement of a pneumothorax.

FIG. 23C provides a closer side view of a surgical chest dome in use.

FIG. 23D shows the superior, that is, the upper, or head-ward, surface of the surgical chest dome.

FIG. 23E shows the inferior, that is, the lower, or foot-ward, surface of a surgical chest dome.

FIG. 24 shows an anterior, or frontal, full-face view of a heart cage for the protection of the heart from tamponade and the inflow and outflow vessels from compression following a metered switch heterotopic transplant of a second heart positioned outside the ribcage or elsewhere not shielded by bone.

FIG. 25A shows a common, internal, and external carotid artery bypass device for use during a carotid endarterectomy, which can if necessary be used to replace the carotids or remain as a bypass so that the flow of blood can be switched from the native carotids through the prosthesis, shown with the valves enlarged for visual clarity.

FIG. 25B shows a common and internal carotid artery bypass device for use during a carotid endarterectomy which can if necessary be left in place to allow the switching of blood flow from the native carotids through the prosthesis, the valves enlarged for visual clarity.

FIG. 26A provides a vertical section side view of a subcutaneously implanted subsurface body port not requiring an outflow opening as does a urological port, with multiple openings for the replenishment by injection of drugs into their respective implanted drug reservoirs.

FIG. 26B is a rear view of the subcutaneously implanted subsurface body multiport shown in FIG. 26A.

FIG. 26C shows a body subsurface port with both a subcutaneous drug inlet and central above-skin scope inlet and urine outlet openings.

FIG. 27A shows a front view of a manually operated multiple-hypodermic syringe injector for allowing the simultaneous replenishment of drug reservoirs through a surface multiport such as those shown FIGS. 17A, 26A thru 26C, and 27C, wherein the openings in the surface port are positioned complementary to the injection points of the injector.

FIG. 27B shows a mechanical spring-loaded jet injector (needle-free injector) equivalent to a pneumatic or air multiple jet injector or the double hypodermic syringe injector shown in FIG. 27A.

FIG. 27C shows the small external tattoo markings placed to assure precise alignment of the hypodermic needle or jet injector nozzle with the injection openings in the subcutaneous port.

FIG. 28 is a schematic anterior view of a bilateral automatic urine collection and voiding prosthesis using one-time manually set valves such as those shown in FIGS. 2 and 3 or solenoid-driven valves such as shown in FIGS. 7 and 8 for permanent placement in a patient with a missing or defective lower urinary tract, and therefore lacking urge sensation, to divert the effluent into a synthetic or surgically constructed neobladder, thence or directly into a paracorporeal collection bag, or urinal cinched about a thigh.

FIG. 29 is a more detailed view of a synthetic neobladder, or neoureter confluence chamber, such as that shown in FIG. 28 for catching urine to allow its automatic expulsion by a small thin film pressure gauge triggered turbine into a paracorporeal collection bag, or urinal, when filled.

FIG. 30 provides a schematic anterior view of a bilateral nonautomatic voluntary urinary diversion system of which the wearer retaining urge sensation and affected by nocturia or frequent urination, for example, can adjust either or both sides between full and zero diversion into a neobladder or directly into a collection bag, or urinal.

FIG. 31A provides a front view of a bendable non-valve ductus flow diversion coupling for insertion into the cut end of a transected vessel other than a carotid, jugular, or coronary, or into a ureter for diverting flow into a synthetic shunt or bypass, which view shows the outflow pattern of a drug delivered into the space between the inner and outer tubes through the opening beneath each upwardly convex tissue-engaging prong.

FIG. 31B is a perspectival view looking down at an angle toward the diverter.

FIG. 31C shows a bendable non-valve ductus flow diversion coupling kept straight for inline use.

FIG. 32A shows the initial venous connection between donor and recipient organs with flow passed through the longer upper of two lines by the venous bloodline-switches at either end, this situation corresponding to the lower venous portion in FIGS. 22A and 22B.

FIG. 32B shows the venous connection between donor and recipient organs when the bloodline-switches have been switched upward to cut off the longer bloodline and pass blood flow through the shorter bloodline, the longer of the two lines then removed.

FIG. 32C shows that blood flow having been diverted to bypass the diseased native organ of the recipient, that organ is removed and the donor organ, or graft organ moved into orthotopic position by transfer and rotation into the circulatory system of the recipient in preparation for anastomosis to the venous stump of the recipient following removal of the diseased organ.

FIG. 32D shows the status in transplantation when the graft organ of the donor has been repositioned as indicated in FIG. 32C.

FIG. 32E shows the configuration of the venous connection upon retraction of the diversion chutes in both inlet and outlet venous vascular valves allowing uninterrupted flow straight through the venous drainage line following anastomosis, so that the expandably structured short line and bloodline-switches can be removed.

FIG. 32F shows that with uninterrupted flow as per FIG. 32E, a permanent line can replace the expandably structured, bloodline-switches, and the vascular valves extended to pass the flow of blood, here venous, through the permanent line to bypass the anastomosis, allowing it time to heal or to clear the native path without the flowing through of blood to interfere with therapy.

FIG. 32G provides a more detailed view of a bloodline-switch.

FIG. 33A provides a closer schematic view of a rotating stopper bloodline switch at the outset of use when blood flow is returned from the donor to the recipient through longer line 170.

FIG. 33B shows how once the donor and recipient have been brought into closer proximity so that the lines connecting their corresponding vessels can be shortened, the stopper at the venous end, just as that at the recipient end, is rotated upward to switch the return of venous blood to the recipient from the longer line 170 to the shorter line 174.

FIG. 34A provides an overhead view of a bloodline switch key which allows simultaneous switching from the longer to the shorter bloodline shown in FIGS. 32A thru 32C of the reverse to accommodate growth, for example, without causing a transient change in blood pressure sufficient to induce a vasospasm and/or a dysrhythmia when dealing with vessels which ought not be clamped, such as the internal carotids, jugulars, and vessels of the heart.

FIG. 34B provides a detailed view of the bloodline-switch key detent mechanism, part number 188 in FIG. 34A, wherein detent 188 is positioned just beneath, or as shown here embedded within, and rotated by bloodline-switch key control knob, part number 179, to simultaneously rotate the polygonal bolt head-like rotating heads and bloodline-switch flapper-type flow stoppers 175.

FIG. 35A shows a confluence conduit which allows the venous return output of the donor organ, hence input into the confluence conduit, of the veins rather than the arteries as exemplary of a donor organ to flow through it together and outlet to the same or a different number of corresponding vessels in the circulatory system of the organ recipient.

FIG. 35B shows a long-to-short of short-to-long switchable confluence conduit which allows the input of one or more vessels of donor organ to flow together through a longer confluence conduit which can be switched by means of a bloodline switch such as that shown in FIG. 32A to a shorter confluence conduit, the long conduit then removed, so that the shorter conduit outlets into the same or a different number of corresponding vessels of the native organ of the recipient.

FIG. 35C shows the initial connections of a donor organ veins by means of vascular valves through a confluence conduit and into the veins of the corresponding native organ to be replaced.

FIG. 35D shows the antepenultimate first step in solid organ transplantation for using the bloodlines of a confluence conduit as bypasses around each anastomosis as shown in FIGS. 22B and 35F.

FIG. 35E shows the penultimate second step in solid organ transplantation for using the bloodlines of a confluence conduit as bypasses around each anastomosis as shown in FIGS. 22B and 35F.

FIG. 35F shows the final step in the use of a confluence conduit in solid organ transplantation for using the bloodlines of a confluence conduit as bypasses around each anastomosis as shown in FIGS. 22B and 35F.

FIG. 36A shows an inline coupling jacket-connected growth compliant expandable conduit which might be a simple tubular tie-line, an anatomically shaped tie-line type prosthesis shaped to include branches for use as a prosthetic replacement for a malformed or diseased segment along a native ductus such as the aortic arch, this view serving also to situate the cross-sections to follow.

FIG. 36B provides a full-face, or face-on, view of the transection indicated in FIG. 36A which has been slightly shifted to show both the accessory channel 8 and an atherosclerotic degeneration preventing perforations 40, which are actually larger and more numerous.

FIG. 36C meant to duplicate FIG. 37C in a structural rather than a functional context provides a full-face or face-on view of the entry inline coupling jacket substrate vessel transection mechanism affixed to the inlet surface of the tie-line cinching inner half-jacket pointed to as part number 231 in FIG. 36A, its exit equivalent 231′ identical but oriented in the opposite direction.

FIG. 36D provides a full-face, or face-on, view of the transection at the level indicated in FIG. 36A.

FIG. 36E provides a full-face, or face-on longitudinal section through an inline coupling jacket-secured line or confluence conduit to clarify its successive surfaces and other parts as specified in section 2b(2)(c)(c1), entitled Inline Coupling Jackets Proper

FIG. 36F shows a schematized inline coupling jacket-secured bridge, or tie-line type anatomical prosthesis to serve as a replacement segment along a malformed, congenitally defective, or severely diseased thoracic aortic arch where the supraaortic branches are likewise secured by inline coupling jackets so that there is one entry jacket at the inline entry and exit termini and four supraaortic exit jackets, all placed automatically.

FIG. 36G shows a tie-line mounted servovalve as exemplary of any one of the different components which can be mounted and fixed in position thus by inline coupling jackets midoperatively without an interruption in blood flow.

FIG. 37A shows inline coupling jackets with outer half-jackets connected to the native vessel in preparation for initiating the rotation into position and the transection and closure by the inner half-jackets to replace the native segment with an autologous, allogeneic, synthetic, or tie-line type prosthetic segment, such as a replacement aortic arch as schematically depicted in FIG. 36F or a tie-line mounted side-entry component such as shown in FIG. 36G.

FIG. 37B depicts the placement of a longer line, tie-line type prosthesis, or tie-line mounted device, which greater in length than the native segment, shares the ability to change the length of the synthetif segment as can bloodline switches such as shown in FIGS. 32A thru 32C.

FIG. 37C duplicates FIG. 36C in support of the functional rather than the structural discussion in the specification.

FIG. 37D shows the through-and-through cross-stitching through the openings 40 of the outer half-jackets of the securing inline coupling jackets connected to the stumps at either end used to fix the prosthesis in position to prevent the tie-line from migrating.

FIG. 37E is a diagrammatic representation of the train of components comprising the mechanism provided to initiate the automatic segment replacement process which uses vibration from a miniature oscillation generator or miniature off-axis mounted cellular or mobile phone-type motor to trip the detent that frees the serrated native ductus cutting razor shown in FIGS. 36C and 37C to descend.

FIG. 37F provides a somewhat more pictorial side view of the native ductus cutting mechanism of FIG. 37E having miniature reciprocating saw type serrated razor cutting edge part number 226 along the lower edge and indent detents 229 along the upper edge to retain the prosthesis half-jacket trip 223 until dislodged and thus freed to descend under the restorative force of the inner half-jacket spring loaded hinge.

### SECTION 6. DESCRIPTION OF THE PREFERRED EMBODIMENTS OF THE INVENTION

### Vascular Valves and Servovalves in General

From the standpoint of pertinence to medical practice, the structure and function of the devices shown in the drawing figures were described in the preceding sections. To allow the internal structures to be shown clearly, vascular valves and servovalves have been drawn disproportionately large, prompting an impression of excessive size and weight. To minimize the risk of patient discomfort and facilitate confirmation of proper function, the vascular valves or servovalves used in any application are always to be as transparent, light in weight, and small in size as possible.

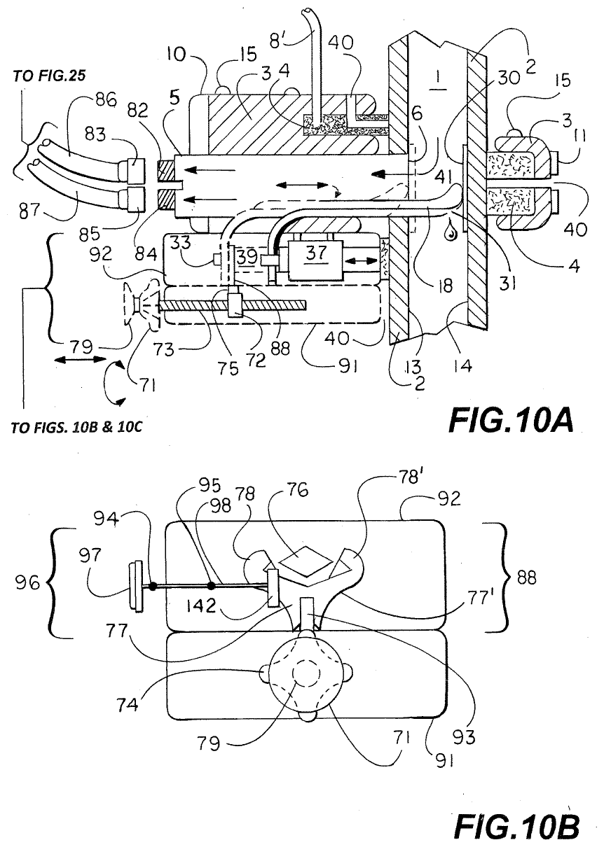

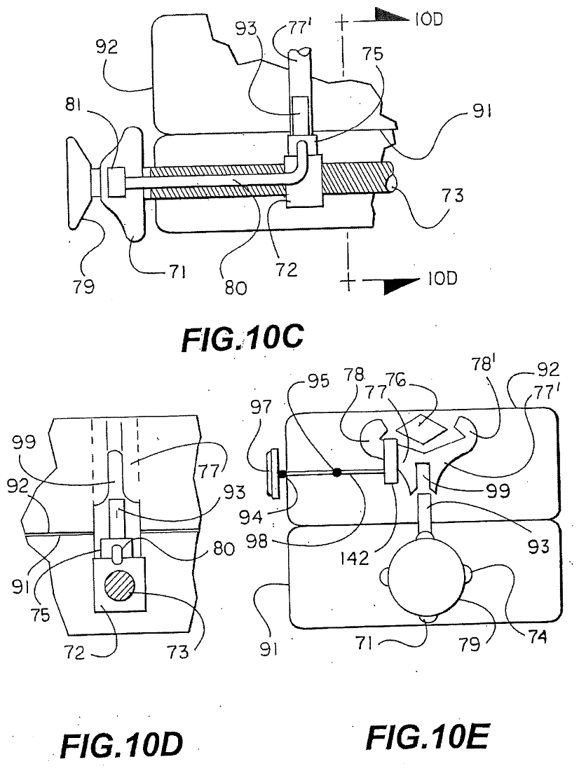

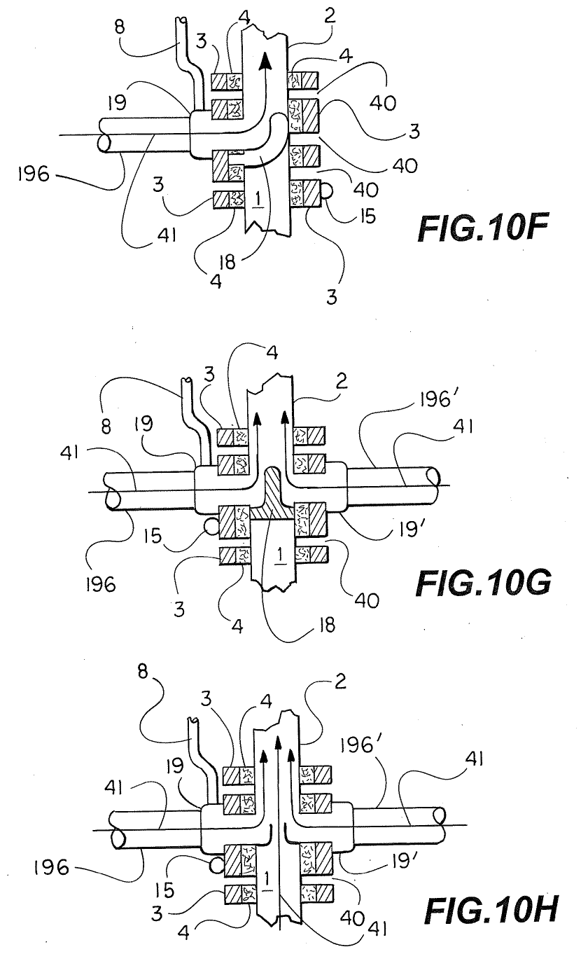

Even though not all of the features incorporable into vascular valves are likely to be needed for any one given application, each type valve has been shown with most if not all of the features necessary to deal with almost any application. That the servovalve shown in FIG. 10A could also use a lead screw that rotates proportionally to the amplitude of the control signal is considered obvious. As addressed below in this subsection, valves and servovalves used with a growth compatible expandable line or conduit must have sufficient pliancy to accommodate growth as well.

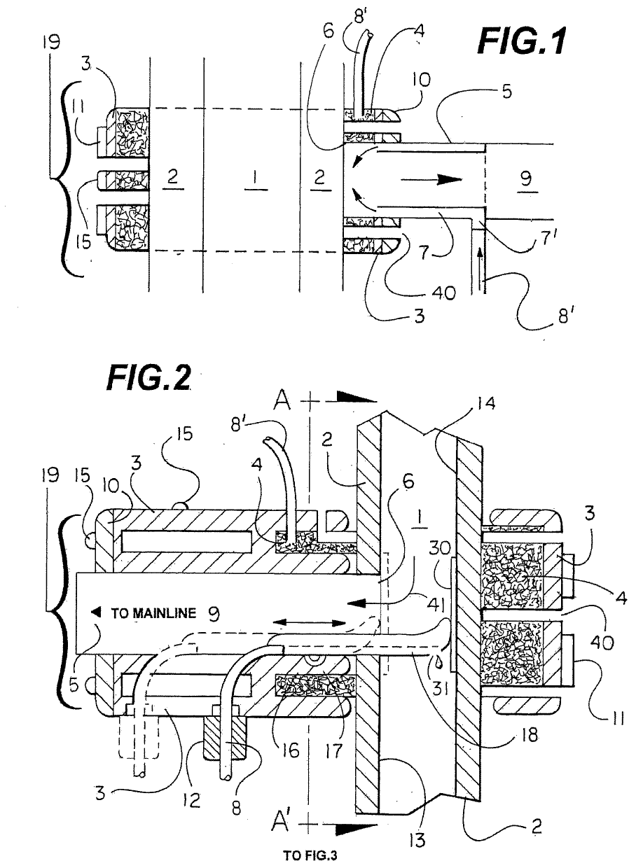

FIG. 1 shows s conventional side-entry jacket as described in U.S. Pat. No. 11,759,186, entitled Ductus Side-entry Jackets and Prosthetic Disorder Response Systems. Bistable in being either fully open or fully closed, vascular valves are essentially side-entry jackets driven by a highly damped nonsparking linear plunger solenoid usually controlled by a suitably encapsulated implanted microcontroller chip switch from uninterrupted flow through the substrate ductus or outflow or effluence through a prosthetic outlet channel, or bloodline, for inflow into another vessel.

In FIG. 2, part number 1 is the lumen, usually vascular or ureteral, 2 the luminal wall, 3 the outer shell of the diversion jacket, 4 viscoelastic polyurethane foam, 5 the trepan tube, and 6 the sharpened trepan leading edge of trepan tube 5. A water jacket is omitted as not required for placement, and an accessory channel not prescribed, diversion chute handle 8 connects to the rear end of flow diversion chute 18. When direct drug targeting is prescribed, accessory channel 8, a hollow capillary tube seen in drawing figures to follow, continues forward where diversion chute handle 8 is connected. Numerous apertures 40 entirely through outer shell 3 and foam lining 4, most outside the plane of the drawings, assure that the adventitia is not completely enclosed as would quickly induce atherosclerotic degeneration in the intima of the substrate ductus.

Accessory channel 8 would then move along the midline underside of diversion chute 18 and terminate in an aperture on the underside distal tip, or nose, of flow diversion chute 18, positioned to release a crystal solvent or drugs into ureteral lumen 1 to wet down urothelial lining 13 and 14 and then enter the bladder. Diversion chute 18 fully extended as shown, positive detent element 16 is engaged within receiving element 17. A mainline—in this case, an outflow catheter or neoureter, connected to the back end of trepan tube 5—is not attached. Diversion chute 18 is made of a material resistant to the adhesion of crystal, so that for many applications, an accessory channel will not be necessary.

Accordingly, when it is desired to target medication directly into the substrate ureter for passage down into a diseased urinary bladder, fixed as well as valvular ureteral takeoff jackets incorporate an accessory channel 8 rather than a diversion chute handle 8, that in the diversion jacket fixed in position once placed. However, where the bladder with the aid of medication is expected to recover, adjustable rather than nonadjustable diversion jackets should be used. Flow into the bladder rather than into a neobladder or collection bag is through nonjacketing side-entry connectors as described and illustrated in U.S. Pat. No. 11,013,858, entitled Nonjacketing Side-entry Connectors and Prosthetic Disorder Response Systems.

Valves for placement along a ureter such as that shown in FIG. 2 are connected when the ureter is passing little if any urine and therefore pose no problem comparable to that encountered with diversion jackets and valves meant for placement along a vessel, where unless prevented, blood would run out. In basic ductus side-entry jackets such as that shown in FIG. 1, a water jacket 8 is used to restrain blood from outflow by directing a jet of water under sufficient pressure to prevent bleeding over the opening or ostium cut in the side of the ductus, typically, an artery where the blood pressure and pulse would pump blood out through the opening.

Once the jacket has been placed, accessory channel 8 makes possible the direct pipeline targeting of drugs into the jacket and the native lumen it encircles. In a urinary diversion jacket, where placement follows voiding so that no outflow occurs, or where some negligible extravasation from the substrate vessel is readily aspirated and unproblematic, a water jacket is omitted. However, when placed in conjunction with an implanted automatic disorder response system able to directly pipeline target drugs to the jacket automatically according to the prescription-program of the implanted microcontroller, one or more incorporated accessory channels is always present.

To cut the side-entry opening, or ostium, into the ureter, the trepan is moved forward with the chute retracted. As distal terminus of the mainline, the trepan is manipulated by loosening lock nut 10, freeing the trepan to be moved reciprocally or rotated. Once the opening has been cut, trepan 6 is retracted into alignment with the proximal urothelial surface, lock nut 10 used to fix it in position, and chute 18 deployed. To prevent jamming or sticking of diversion chute 18 when deployed after the jacket has been positioned, the segment at the distal end of the mainline with trepan distal edge can be made of low friction fluoropolymeric tubing, such as polytetrafluoroethylene.

To prevent backfilling with blood and the possibility of obstruction by clot, druglines 8 and 8′ along synthetic or organic bloodlines, or blood conveying passageways can be filled with water and/or provided with an anticoagulant drip. The preceding specification addresses numerous coatings and agents which can be sent to a jammed device to dispel clot, biofilm, and the accretion of crystal, for example. The accessory channel or sideline catheter is made of a polymer such as polytetrafluoroethylene in a thickness as will bend only so much as to allow the operator to advance the diversion chute into the ureteric lumen.

Where there is the potential for kinking, softer more pliant polymeric tubing is used. If urging by edge of the entry hole to the side removed from the ureter at the bottom of the jacket is insufficient to guide the accessory channel tube forward, a small curved quarter round guide is bonded to that edge to assure smooth and properly aligned movement of the service channel tube through the entry hole. Sideways bendability of this material is, however, sufficient as not to interfere with any slight rotation or reciprocation of the trepan the operator may need to create the opening into the ureter.

### Expandable Vascular Valves and Servovalves

Vascular valves and servovalves used with expandable lines and conduits as addressed above in section 2b(2)(f), entitled Expandable Structure to Accommodate Growth and Self-adjust in Size in Vascular Segment Prostheses, Lines, and Confluence Conduits Placed in Children, must also accommodate growth. The selection of a pressure adaptable and compliant implantable plastic or pliant spongy and implantable material must have durability consistent with the application and a relatively high plasticizer content which must be completely washed away from the surface.

Materials currently available that exhibit the required characteristics are relatively few (see, for example, Lee, H., Kim, H. J., Shin, Y., and Kim, D. H. 2024. “Phase-separated Stretchable Conductive Nanocomposite to Reduce Contact Resistance of Skin Electronics,” Nature Scientific Reports (London, England) 14(1): 1393; Lienemann, S., Boda, U., Mohammadi, M., Zhou, T., Petsagkourakis, I., Kim, N., and Tybrandt, K. 2024. “Exploring the Elastomer Influence on the Electromechanical Performance of Stretchable Conductors,” Online, ACS [American Chemical Society] Applied Materials and Interfaces 16(29): 38365-38376; Yu, H., Hu, Z., He, J., Ran, Y., Zhao, Y., Yu, Z., and Tai, K. 2024. “Flexible Temperature-Pressure Dual Sensor Based on 3D Spiral Thermoelectric Bi₂Te₃ Films,” Online, Nature Communications (London, England) 15(1): 2521; Bains, P. and Kaur, S. 2023. “Silicone in Dermatology: An Update,” Online, Journal of Cutaneous and Aesthetic Surgery (Mumbai (Bombay), India) 16(1): 14-20; Sunwoo, S. H., Han, S. I., Jung, D., Kim, M., Nam, S., and 10 others 2023. “Stretchable Low-impedance Conductor with Ag—Au—Pt Core- Shell-Shell Nanowires and in Situ Formed Pt Nanoparticles for Wearable and Implantable Device,” ACS [American Chemical Society] Nano (Washington, DC) 17(8): 7550-7561; Wang. H. 2023. “Biomaterials in Medical Applications,” Online, MDPI [Multidisciplinary Digital Publishing Institute] Polymers (Basel, Switzerland) 15(4): 847; Hu, G., Wang, S., Yu, J., Zhang. J., Sun, Y., and Kong, D. 2022. “A Facile and Scalable Patterning Approach for Ultrastretchable Liquid Metal Features,” Lab on a Chip (Cambridge, England) 22(24): 4933-4940; Lim, C., Park, C., Sunwoo, S. H., Kim, Y. G., Lee, S., and 5 others 2022. “Facile and Scalable Synthesis of Whiskered Gold Nanosheets for Stretchable, Conductive, and Biocompatible Nanocomposites,” ACS [American Chemical Society] Nano (Washington, DC) 16(7): 10431-10442; Wang, S., Nie, Y., Zhu, H., Xu. Y., Cao. S., and 5 others 2022. “Intrinsically Stretchable Electronics with Ultrahigh Deformability to Monitor Dynamically Moving Organs,” Online, Science Advances (Washington, DC) 8(13): eabl5511; Eckrich, J., Hoormann, N., Kersten, E., Piradashvili, K., Wurm, F. R., and 5 others 2021. “Surface Modification of Porous Polyethylene Implants with an Albumin-based Nanocarrier-release System,” Online, MDPI [Multidisciplinary Digital Publishing Institute] Biomedicines (Basel, Switzerland) 9(10): 1485; Hussain, T., Gellrich, D., Siemer, S., Reichel, C. A., Eckrich, J., and 4 others 2021. “TNF-α-Inhibition Improves the Biocompatibility of Porous Polyethylene Implants in Vivo,” Online, Tissue Engineering and Regenerative Medicine (Seoul, South Korea) 18(2): 297-303; Joo. H., Jung, D., Sunwoo, H., Koo. J. H., and Kim, D. H. 2020. “Material Design and Fabrication Strategies for Stretchable Metallic Nanocomposites,” Small (Weinheim an der Bergstrasse, Germany) 16(11): e1906270; Choi, S., Han, S. I., Jung, D., Hwang, H. J., Lim, C., and 14 others 2018. “Highly Conductive, Stretchable and Biocompatible Ag—Au Core-sheath Nanowire Composite for Wearable and Implantable Bioelectronics,” Nature Nanotechnology (London, England) 13(11): 1048-1056; Czuba, L. 2014. “Application of Plastics in Medical Devices and Equipment,” Online, in Modjarrad, K. and Ebnesajjad, S. (eds.), Handbook of Polymer Applications in Medicine and Medical Devices, 2014:9-19).

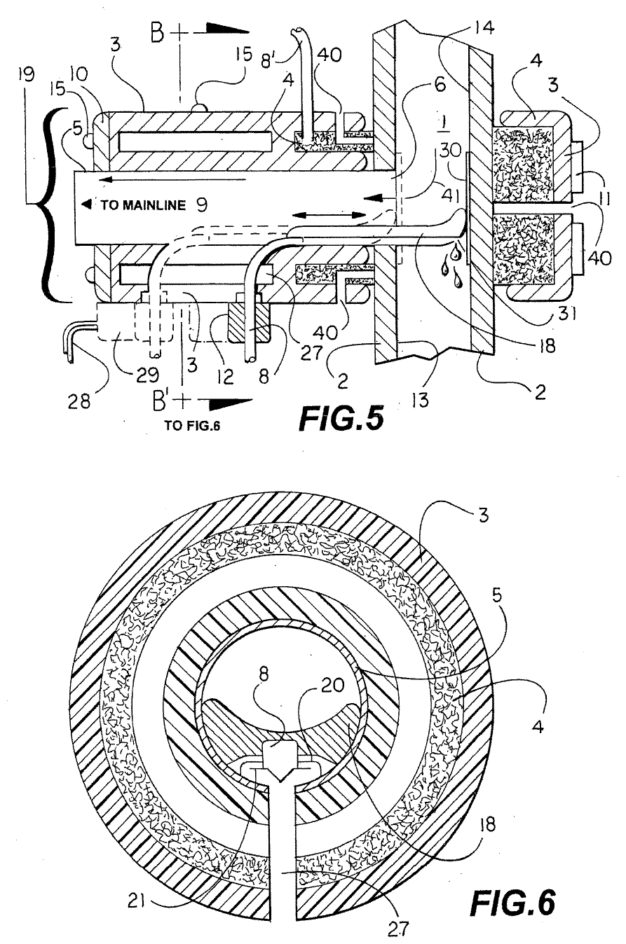

Despite the use of an aspiration or suction pump as described above in section 2a, entitled Background to the Invention. General, if necessary, trepan 6 can be forcibly rotated to expedite cutting the opening into the native lumen. Accessory channels 8 and 8′ can be made of any implantable catheter synthetic. In the manually operated valves shown in FIGS. 2 and 5, accessory channel 8 (not 8′ which enters from above) courses through and along the bottom of flow diversion chute 18 to continue distad up to the underside exit pore 31 of accessory channel 8, here equal to the accessory channel. In the urinary assist embodiments shown in FIGS. 28 and 30, flow diversion chute 18 is advanced (extended) into the lumen 1 of the substrate ureter manually by pushing accessory channel 8 forward or pulling it backward.

The implanted controller adjusting the diversion chutes, water fill and the release of an anticoagulant are coordinated automatically, various valve types are shown in FIGS. 1 thru 3, 5 thru 8, and 10 thru 14. In FIG. 1 showing a basic side-entry jacket, number 1 is the lumen of the substrate ductus to be jacketed, and 2 the wall surrounding the lumen, both products of nature and not part of the invention.

Part number 3 is the outer shell of the basic side-entry jacket, usually made of polyether ether ketone, 4 a viscoelastic polyurethane foam layer lining shell 3 containing perforations along its length to avert atheromatous degeneration of the encircled ductus. The adventitia-protective open cell viscoelastic polyurethane foam lining the valve-jackets shown in FIGS. 1, 2, 3, 5, 7, 8, and 10A are thoroughly wetted with an anti-inflammatory, immunosuppressive, and/or antimicrobial upon placement which is periodically replenished through accessory channel 8′.

Represented entering perpendicularly into the foam for visual clarity, entry is equally effective when the proximal end of accessory channel 8′ is inserted along perforation 40 running about the circumference of side stem 19, shell 3 necessarily interrupted by solid segments for structural integrity. Part number 5 is the trepan tube with razor sharp leading front edge 6, which in a basic side-entry jacket, unlike a diversion valve jacket, requires no stationary surrounding tube to house and align driving means such as a miniature highly damped nonsparking solenoid or linear servomotor, so that it alone comprises jacket side-stem 19. Features common to all valves are that to eliminate abrasive and pressing against or poking into neighboring tissue, all corners and edges are rounded.

Little heat is generated or conducted through the enclosure of the servomotor to surrounding tissue, which is unlikely to come into contact with the valve anyway. If necessary, the entire valve is further enclosed within a sock made of a soft biocompatible thermally insulating material to provide both increased freedom from abrasive encroachment upon neighboring tissue as well as reduce any heat which the servomotor might generate. The basic ductus side-entry jacket shown In FIG. 1 lacks a magnetic envelope to draw superparamagnetic micro- or nanoparticle carrier-bound drugs from the passing luminal contents and/or a radiation shielding envelope sufficient to protect surrounding tissue against moderate dose-rate radionuclides, for example.

In comparison with vascular valves, vascular servovalves are essentially side-entry jackets which driven by a servomotor and usually controlled by a suitably encapsulated implanted microprocessor, can draw off a continuously variable proportion of the flow through a ductus to switch from uninterrupted flow through the substrate ductus, or outflow, or effluence, through a prosthetic outlet channel, or bloodline, for inflow into another vessel. Another means for the continuously variable of such a diversion valve is directly manual such as pertains to the valves shown in FIGS. 2, 5, and 30 where the wearer of a urinary assist device can adjust the volume of flow into either of two thigh-bound collection bags.

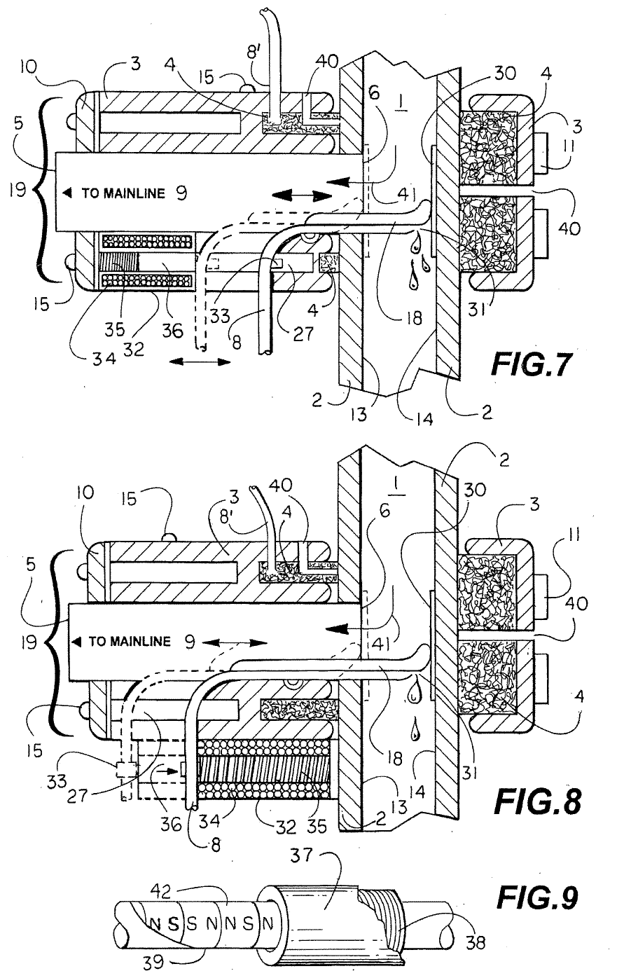

FIG. 7 shows an inline plunger solenoid driven bistable vascular valve that ejects, that is, pushes diversion chute 18 forward, while the vascular valve shown in FIG. 8 pulls chute 18 forward. As are all side-entry devices, despite the impression created by the intentionally enlarged drawings, the actual sizes thereof are matched to the size of the substrate ductus. The valve shown in FIG. 2 is directly, that is, manually adjusted by the operator upon placement. That shown in FIG. 5 is remotely adjustable, here by means of push/pull, or Bowden cable, used in the implanted, hence, inaccessible urinary assist device shown in FIG. 30.

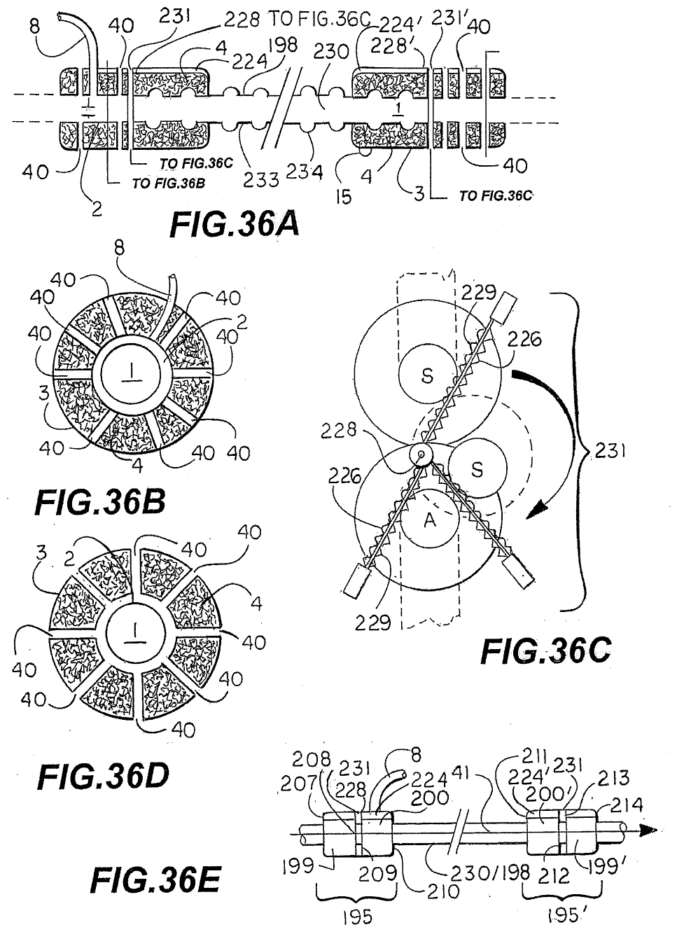

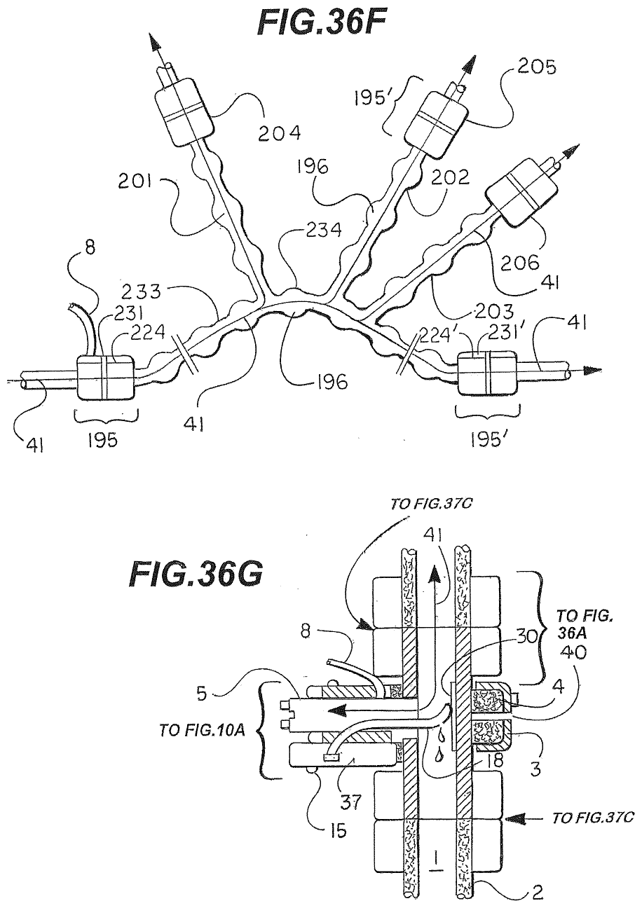

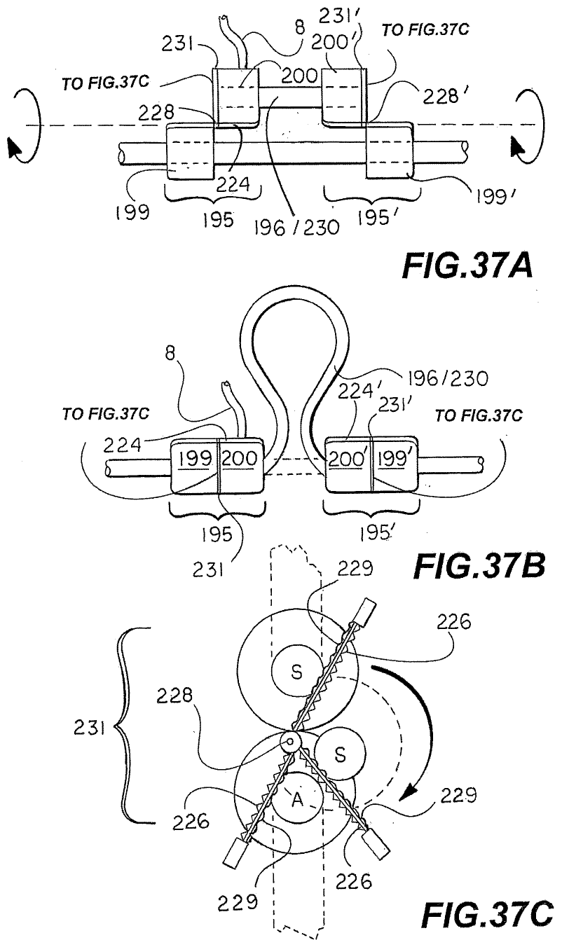

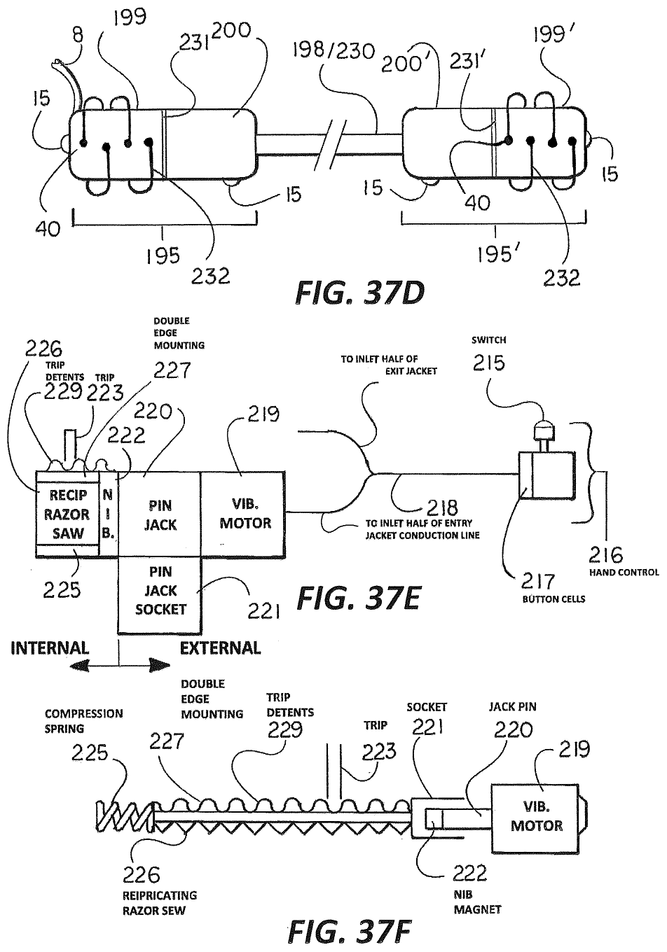

The protrusion of the sidestem 19 an intrinsic factor in vascular valves, the diversion chute pusher valve in FIG. 8 allows the driver part of the valve at the bottom to be shorter. However, the edges and corners of these valves are rounded, can have a layer of cushioning such as with a soft silicone rubber applied, and can be positioned at any level available at any rotational angle along the substrate ductus. If to avoid protrusion, a more distant level is wanted, a tie-line mounting as shown in FIG. 36G can be used to avoid protrusion into neighboring tissue. Tie-line mounting of flow directing components is addressed below in section 2b(2)(e), entitled Tie-line Mounted Side-entry Flow Diversion Jackets, Vascular Valves, and Servovalves.

Where the need for adjustability in flow volume is not a factor, inline coupling jackets as addressed above in sections 2b(2)(c), entitled Vascular Segment Prostheses: Inline Coupling Jackets and Bridging, or Tie-lines: 2b(2)(c)(c1), entitled Inline Coupling Jackets Proper; and 2b(2)(c)(c2), entitled Automatic Insertion into a Ductus of an Inline Coupling Jacket-secured Bridging, or Tie-line and Its Replacement if Necessary, and shown in FIGS. 36A, 36E, 36F, 37A, and 37F can be used. FIG. 9 shows a linear servomotor suitable for use as the driver in a vascular servovalve. FIGS. 10A thru 10E include a fine manual intervention adjustment device described below for applying fine adjustments in the distance, speed, and direction of diversion chute 18 deployment during its extension or retraction.

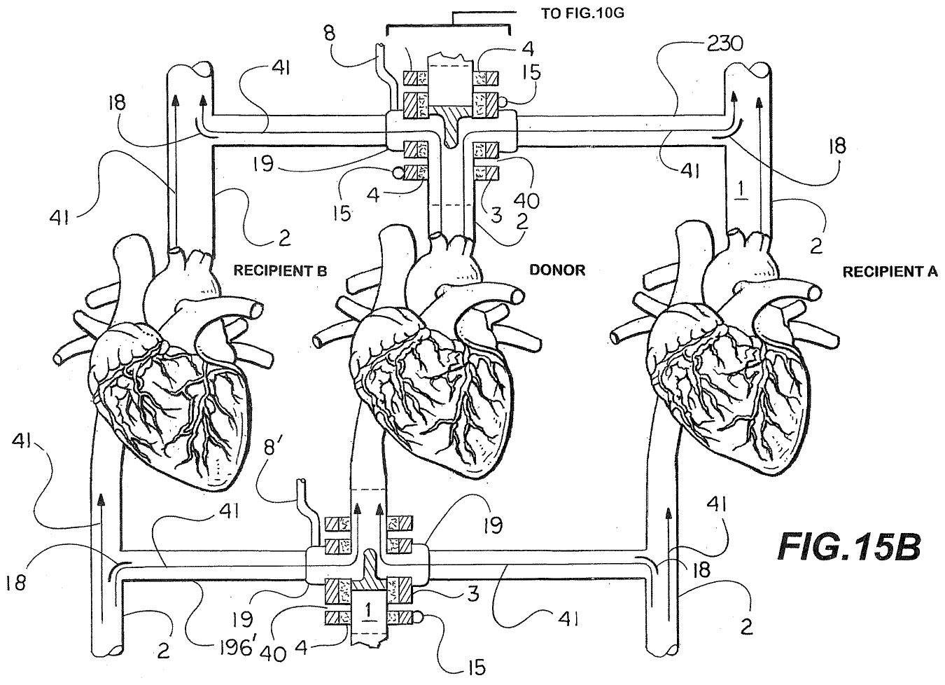

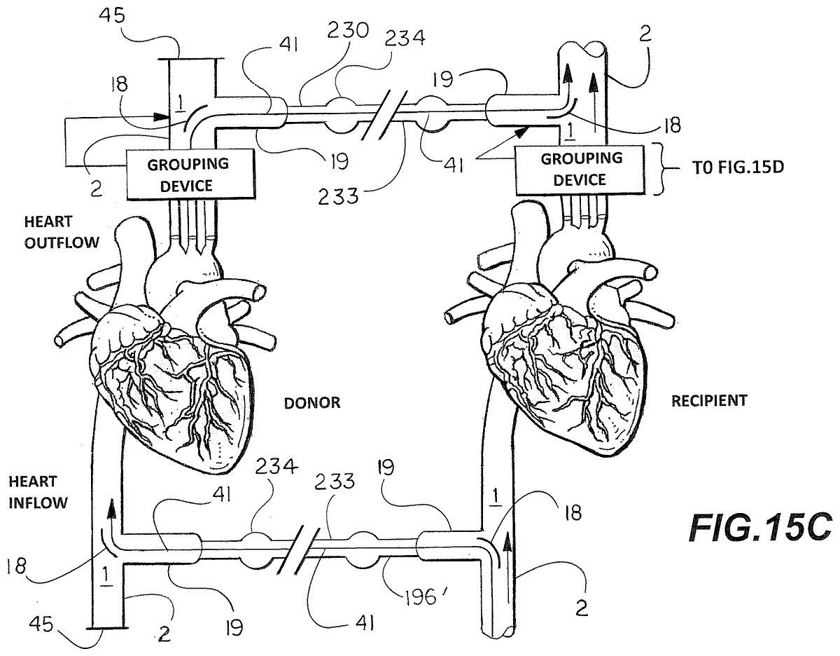

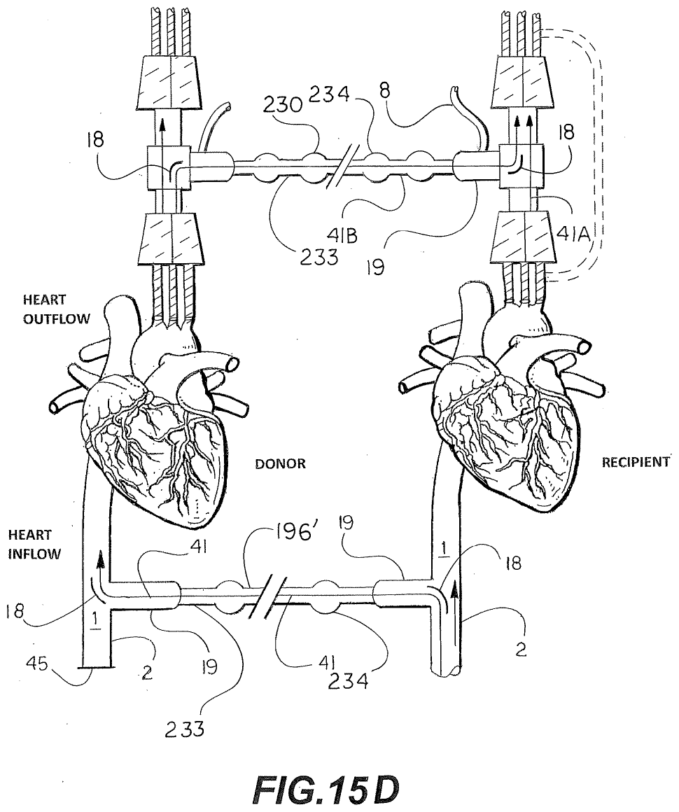

FIGS. 15C and 15D provide views of expandable confluence conduits 230 at the top and individual vena caval bloodlines 196 at the bottom, while FIG. 36A provides a longitudinal sectional view of a simple tubular tie-line and 36F a view of an anatomical tie-line type prosthesis incorporating the expandable structure for use in a growing child described above in section 2b(2)(f), entitled Expandable Structure to Accommodate Growth and Self-adjust in Size in Vascular Segment Prostheses, Lines, and Confluence Conduits Placed in Children. FIG. 36E provides a longitudinal sectional detail view of a tie-line, whether tubular, anatomical, or expandable incorporating the flats 233 and vaults 234 structure for use in growing children described above in section 2b(2)(f), of which the associated nomenclature is specified above in section 2b(2)(c)(c1), entitled Inline Coupling Jackets Proper.

The microprocessor operates under a prescription-program that coordinates inputs from pertinent physiological sensors with the release into the reciprocally cross-circulated or shared bloodstream to govern the rate of diversion chute advancement, and if necessary, reverse or suddenly retract or advance the chutes temporarily or to complete the transfer of the graft organ from the donor to the recipient. Numerous miniature linear motor types can be used to accomplish closed circuit control over the speed and direction of diversion chute deployment, or advancement of the chutes into the substrate native lumina.

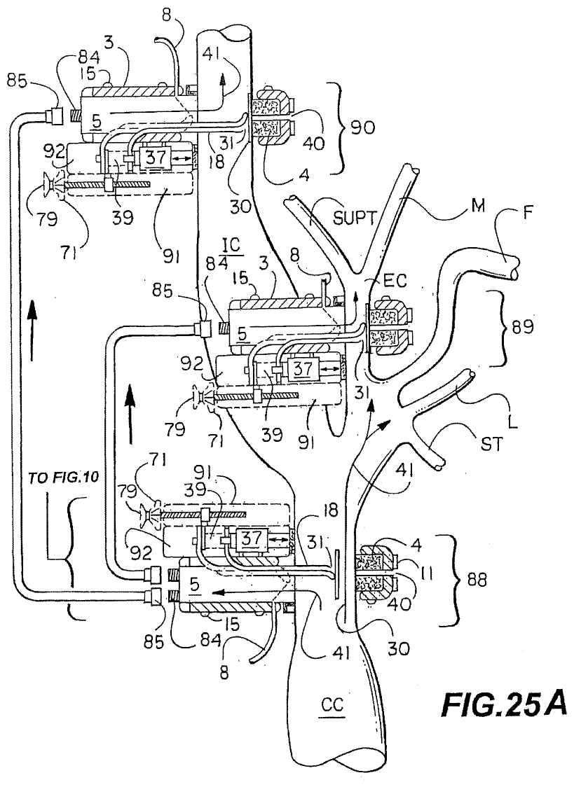

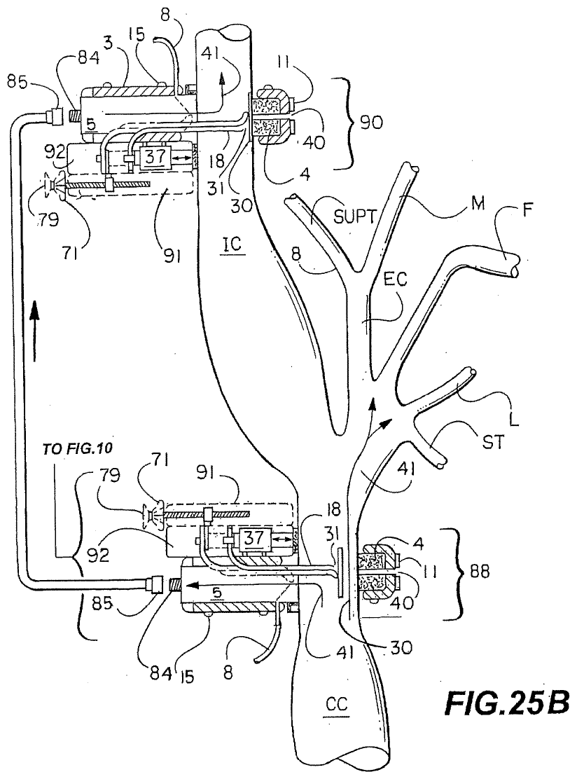

Valves of which the function would best allow direct observation by the operator of their internal function are made of a clear transparent plastic which provides thermal insulation as well. Suitable polymers include those biocompatible based upon poly(aryl-etherethereketone) or PEEK, polyethylene, and polyethylene terephthalate, for example. To facilitate endoscopic examination should the need arise, valves are best made with a polymer of high optical clarity. Vascular servovalves used to create a compound vascular bypass to transfer a solid organ from the donor to the recipient are shown in FIGS. 10A, 25A, and 25B, FIGS. 10A thru 10E, 25A, and 25B also showing the addition of the direct manual fine control device that allows the operator to make small shifts in flow diversion chute position to assure complete obturation of the ostium by the ostium obturator as necessary.

For example, when the diversion chute is passed through the ostium created in the side of the substrate ductus, the obturator (ostium obturator, stopper) is likely to fully open a tiny distance from the proximal wall of the ductus through which it was inserted. Enclosed within clear transparent plastic, the direct manual fine control adjustment device allows the operator to retract the ostium obturator that tiny distance so that the ostium obturator rests flush against the ostium, thus preventing blood from exiting through the ostium and into the protective foam or the trepan tube.

Unless an open surgical field is necessary for unrelated reasons, vascular valves are placed endoscopically. Part number 6 is the razor-sharp leading edge of the surgical circle cutter, or trepan itself, of trepan tube 5. Lacking the ductus side-entry opening, or ostium, obturator, of a valve jacket, a basic side-entry jacket minimizes bleeding when the tissue plug is extracted by directing a circular jet of water over the ostium through water jacket 7 fed through water jacket inlet 7 sideline, and accessory channel, 8.

In any instance where the valve or servovalve jacket accessory channel or channels incorporated into the jacket such as those shown in FIGS. 2, 5, 7, 8, and 10 numbered 8 or 8′ are insufficient to deliver the volume or number of drugs sought to be directly pipeline targeted to the treatment site, a separate basic ductus side-entry jacket is positioned upstream to make up the difference. Immediately after the jacket has been placed in position, accessory channel 8 becomes available for delivering drugs, for example.

The ostium is cut by a suction pump connected to trepan tube 5, the suction passing through the luminal continuum comprising an attached catheteric line, or mainline 9 connected to trepan tube 5 in the side stem 19. Trepan tube 5 is encircled and travels within side-stem 19, absent an accessory channel, part number 8 is a small handle to advance diversion chute 18; when an accessory channel is present, 18 is its conduit. The application of suction against ductus wall 2 through trepan tube 5 draws ureteral wall 2 past trepan 6, excising a tissue plug from the side of ureteral wall 2. The suction pump has a limit switch that instantly stops the pump when the resistance to suction suddenly drops off and the water jet minimizes bleeding while continued suction and if necessary, a guidewire with hooked distal end is used to extract the plug.

If for any reason-such as an hydroxyapatite plaque cap is encountered-plug excision is resisted, the operator can loosen locking nut 10 to rotate and reciprocate trepan tube 5. Once trepan 6 is aligned to the inner surface of ductus wall 2, locking nut 10 is tightened to lock trepan tube 5 in position. Used medically, vascular valves and servovalves are unique in various details of internal construction, shape factor, method of placement, and accessory channels, but not in terms of driving means, which are miniaturized versions of conventional linear plunger solenoids or tiny linear servomotors.

Fixed in conformation and noncontrollable or adjustable and limited to use as static or passive flow diversion or flow directional guides, passive one-, two-, and three-way side-entry diversion jackets, addressed above in section 2b, entitled Vascular Valves and Servovalves in the Diversion of Blood Flow, the subsection thereof entitled One-, Two-, and Three-way Passive Side-entry Flow Diversion Jackets shown in FIGS. 10F thru 10H and in its primary application shown in FIG. 15B, have no driving means. FIG. 10F shows a one-way, FIG. 10G, a two-way, and FIG. 10H a three-way passive side-entry flow diversion jacket.

The jackets of these conform in structure to the descriptions provided for side-entry jackets in U.S. Pat. No. 11,389,171, entitled Integrated System for the Infixion and Retrieval of Implants, U.S. Pat. No. 11,759,186, entitled Ductus Side-entry Jackets and Prosthetic Disorder Response Systems, as well as above in section 2a, entitled Background of the Invention. General, to include the preferability of transparent polypropylene or polyetheretherketone polymer, and as shown in FIGS. 10F thru 10H, a lining of a highly compliant nonfragmentable dissolution-prevention coated viscoelastic polyurethane foam 4, at least one accessory channel into which a drug delivery line 8 can be inserted, and multiple perforations 40 entirely through to the substrate adventitia 2.

Fixed in internal conformation, nonadjustable, and therefore not valves, simple one-, two-, and three-way side-entry diversion jackets or flow directional guides are often an essential complement to vascular valves and servovalves for continuing the direction of flow entered through a vascular valve or servovalve. Confluence conduits can be both entered and exited by one-way flow directional guides. All applications of these conformant to function thus, passive side-entry flow diversion jackets are virtually inseparable from vascular valves and servovalves. Lacking a laterally protrusive sidestem 19, associated with the need for a translatable flow diversion chute or tongue, these eliminate the midoperative task of finding the optimal level and rotational angle for mounting the device in relatively dense anatomy as enumerated in section 2a, entitled Background of the Invention. General.

The level along the substrate ductus is prepared by placing a catheter with a leading-edge razor-sharp ferrule connected to an aspiration or suction pump against the side of the substrate ductus. This allows a small plug to be drawn out of the wall of the ductus which the operator immediately covers with his thumb to prevent extravasation. The jacket consists of two half semicylinders joined by a joint-traversing spring hinge. The diversion chute is fused to the internal surface of one side of the jacket by means of heat or solvent plastic welding at a point that allows closure of the jacket to project the diversion chute diametrically through the side hole so that its distal tip is flush against the internal surface of the opposing half-jacket.

The jacket half-cylinders lock together by means of a notched or detented ratchetless pawl at the center edge of one half-jacket with its receiver at the center edge of the opposite half-jacket. The operator advances the pawl notch by notch until the distal tip of the diversion chute is flush against the surface of the foam lining. In numerous applications, especially in a compound vascular heart transplant where the number of donor-recipient corresponding vessel connections is high, using these passive flow diversion or flow direction guides reduces the operational duration as well as the cost for the necessary apparatus.

In the vascular tree, these are positioned at the inlets and outlets into bypasses and shunts that require no adjustments in flow rate. Manually operated urinary valves such as used in a ureter-to-collection bag diversion prosthesis usually employ simple mechanical controls for adjustment such as push\pull, or Bowden cables, but can just as well use a remotely controlled highly damped nonsparking solenoid, for example, especially if to route the Bowden cables creates the potential for strangulating intervening tissue.

In order from most to least simple, vascular jackets include fixed flow diversion jackets, manually controlled bistable flow nondiverted-flow diverted valves, such valves and chokevalves when solenoid-driven, and continuously variable servo chokevalves under automatic control by an implanted sensor-fed microcontroller or microprocessor. A sphincteric valve such as that shown below externally encircles to grasp, or cinches about to constrict the substrate ductus from without and is structurally and functionally distinct from other vascular jackets, valves, and servovalves. Nonadjustable, the ureteral takeoff diversion jacket show in FIG. 2 is not a valve, which connotes adjustability, but rather a static prosthesis for placement in a patient with a missing or irreparable urinary tract.

The diversion jacket is shown fully extended with vacuum hose having been disconnected from the rear end of trepan tube 5, after the side-entry tissue plug has been extracted and the diversion chute 18 extended into the ureteral lumen. Nonadjustable, it requires a diversion chute, but since urine passing down the ureter should always flow freely out through the trepan tube and into the catheteric mainline, here referred to and further explained below, as a neoureter, and not accumulate, an ostium obturator, for preventing outflow into the diversion jacket is omitted. In contrast, in a urinary diversion valve which to allow the user to switch from normal urination during waking hours to diversion with no urge sensation as would awaken the wearer during the night, a ostium obturator is necessary.

The valves shown in FIGS. 7 and 8 are driven by linear plunger solenoids comprising electromagnetic coil or winding, 35 and solenoid plunger 36, while that shown in FIG. 10A is driven by a servomotor such as that shown in FIG. 9. All vascular valves, such as those shown in FIGS. 2, 5, 7, and 8, and servovalves such as that shown in FIG. 10A can switch flow from the substrate ductus to pass out through the sidestem 19, the servovalve discretionary in allowing this action as continuously variable. The method for placing a valve or servovalve along a vessel is described above early in section 2a, entitled Background of the Invention. General.

Insertion through a hole in a side of the substrate ductus, ordinarily a blood vessel or ureter, eliminates the need to transect the vessel and interrupt the flow of blood through it. While in most smaller vessels, brief clamping to allow transection without the loss of blood is safe, this is not so with respect to the vessels that supply the brain and heart nor the great vessels that ultimately supply and drain the lungs through the pulmonary circulation and the rest of the body through the systemic circulation. With these and the heart itself, an interruption in perfusion requires cardiopulmonary bypass, in itself, a cause for numerous complications, some potentially serious, which if the need therefor is eliminated, would materially improve outcomes following cardiovascular procedures.

In all the embodiments of valves and servovalves shown, when upon placement, diversion chute 18 with obturator 30, made of a pliant material such as an implantable silicone rubber and molded as a unit or fused together, are pinched narrow to pass through the hole in its side, the thinned out or feathered periphery at the distal ends thereof, shape-compliant, are splayed against the curved interior wall of the ductus cutting off flow past this level (see, for example, Faruq, O., Chien, P. N., Donmez, N., Nam, S. Y., and Heo, C. Y. 2021. “Functionalization of Silicone Surface with Drugs and Polymers for Regulation of Capsular Contracture,” Online, MDPI [Multidisciplinary Digital Publishing Institute] International Polymers (Basel, Switzerland) 13(16): 2731; Li, X., Zhou, X, Chen, Y., Yu, S., Chen, X., and 4 others 2017. “Surface Changes of Nanotopography by Carbon Ion Implantation to Enhance the Biocompatibility of Silicone Rubber: An in Vitro Study of the Optimum Ion Fluence and Adsorbed Protein,” Journal of Materials Science. Materials in Medicine 28(10): 167; Lei, Z. Y., Liu, T., Li, W. J., Shi, X. H., and Fan, D. L. 2016. “Biofunctionalization of Silicone Rubber with Microgroove-Patterned Surface and Carbon-ion Implantation to Enhance Biocompatibility and Reduce Capsule Formation,” Online, International Journal of Nanomedicine (Auckland, New Zealand) 11:5563-5572).

Furthermore, for conducting blood, this material should be treated to repel pathogens, and clot. If necessary, such passive means can be supplemented with such agents through the direct pipe-targeting to the valve and its substrate vessel through drugline or druglines, part numbers 8 and 8′, in the drawings inserted in one or more of its accessory channels. Antimicrobial, thrombophobic, and other protective coatings and inclusions are addressed above in such sections as 2b, entitled Vascular Valves and Servovalves in the Diversion of Blood Flow.

Diversion chute 18 fully extended, all flow is diverted out through sidestem 19. When fully retracted, obturator 30, shown ghosted or with hatched lines, shuts off sidestem 19 from outflow to the condition of zero flow diversion. Whereas the highly damped nonsparking linear plunger solenoids shown allow emergency sudden-switch solid organ transplantation, servovalves, such as that shown in FIG. 10A, make possible the metered transfer of an organ between the circulatory systems of a donor and recipient.

A servomotor of the kind used in the servovalve shown in FIG. 10A appears in FIG. 9, wherein part numbers 37 is the cylindrical motor housing, 38 are adjacent circular electromagnet coils, 39 is the linear servomotor shaft, and 42 are successive pairs of cylindrical neodymium iron boron permanent magnets. At any setting, the diversion valve will remain in place until the servovalve receives another input from the master control microprocessor. Metered transfer involves the continuous automated monitoring by a master control microprocessor for signs of rejection and responding thereto accordingly.

Prosthetic disorder response systems as the means for the control of side-entry devices as end-actuators in an implanted automatic therapeutic system are addressed in copending application Ser. No. 17/689,880, entitled Prosthetic Disorder Response Systems, as well as in U.S. Pat. No. 11,759,186 as wll as in U.S. Pat. No. 11,759,186, entitled Ductus Side-entry Jackets and Prosthetic Disorder Response Systems, and above in this document. The crossmatching involved in either is addressed above in sections 2b(1)(a), entitled Sudden Compound Vascular Bypass, or Sudden Switched-organ, Transplantation, and 2b(1)(b) entitled Metered Compound Vascular Bypass, or Switched-organ, Transplantation, among others.

When used in a prosthesis, such as to divert urine past diseased or missing parts of the lower urinary tract, switching from fully retracted to fully advanced is the final adjustment when the prosthesis is placed. In the urinary prosthesis shown in FIG. 28 meant for a patient lacking a functional lower urinary tract, the simple ductus side-entry diversion jackets are not servovalves, which term denotes continuously variable adjustability. Instead, the prosthesis passively collects urine past diseased or missing parts of the lower tract.

In contrast, for a patient with impaired urinary function such as frequency or incontinence, the urinary aid shown in FIG. 30 requires the ability to switch between use and not in use positions of the diversion chutes 18. The manually adjusted valve shown in FIG. 2 is used by the operator to set the urinary prosthesis embodiment shown in FIG. 28 while that shown in FIG. 5 is used by the patient wearer to adjust the urinary assist embodiment shown in FIG. 30.

FIGS. 2, 5, 7, 8, and 10A all show vascular valves, of which those shown in FIGS. 2 and 5 are adjusted by hand, that in FIG. 2 directly by the operator during placement, while that in FIG. 5 indirectly by the wearer through a Bowden push pull cable to adjust the volume of urine to be stored at any given time from 0 to 100 percent. Those shown in FIGS. 7 and 8 are driven by linear plunger solenoids, that in FIG. 7 in a pushing, while that shown in FIG. 8 in a pulling configuration. FIG. 10A is a continuously variable servovalve controlled by a servomotor of the type shown in FIG. 9.

More specifically, the valves shown in FIGS. 7 and 8 are controlled by highly damped nonsparking plunger solenoids, so that these are limited to bistable, that is, dual state in allowing only complete extension or retraction of the valve flow diversion chute, part number 18, and therefore, only the complete stoppage or passage respectively of flow through either the native or synthetic bypass lumen with no ability to position the chute at an intervening point between full closure and fully open to apportion flow between one volume which is diverted out through its side-stem 19 and the other allowed to continue through the native lumen.

Drawing FIGS. 10F thru 10H show side-entry passive diversion jackets which complement vascular valves and servovalves in directing flow as received from one or more side-entry valves or servovalves. FIG. 10F shows a passive single column flow-redirecting guide addressed above in section 2b, entitled Vascular Valves and Servovalves in the Diversion of Blood Flow in the subsection thereof entitled Simple Flow Diversion Side-entry Jackets. The double half-cylinders of the jacket can be made of polyetheretherketone or polypropylene, and the fixed diversion chute of a suitable silicone elastomer of cross-linked polydimethylsiloxane, for example.

FIG. 10G shows a passive double inflow column flow-redirecting guide addressed above in section 2b, entitled Vascular Valves and Servovalves in the Diversion of Blood Flow in the subsection thereof entitled Simple Flow Diversion Side-entry Jackets. FIG. 15B shows the use of such a side entry double outlet simple flow diversion jacket to allow the gradual transfer of flow between an allograft heart and native heart of a first recipient to a second recipient. Its composition can be the same as the single column flow redirection jacket preceding in FIG. 10F.

FIG. 10H shows a triple inlet passive side-entry flow diversion jacket. Since the heart does not distribute but rather only pump blood, a double and not a triple outflow jacket is needed.

That is, complete extension of the diversion chute in a vascular servovalve denotes diversion of all the fluid flowing through the ductus while partial extension denotes that a fraction of the column divided thus is diverted into another ductus, and the other allowed to continue through the ductus, and complete retraction denotes that flow is not divided but rather allowed to continue through the ductus. In fact, a flow diversion valve in a highly damped nonsparking solenoid-driven bistable valve is never completely ‘closed’ or ‘open’ except with respect to either of its effluence paths, of which one or the other will be fully open or closed. In contrast, continuously variable, the diversion chute in a vascular servovalve can be continuously adjusted between its fully extended and retracted positions.

Thus, at any intervening position, either of its effluence paths will be ‘open’ or ‘closed’ each as the complement to the other within the overall flow-through cross-section of the valve. Accordingly, with the flow diversion chute of any type vascular valve fully extended, the lumen of the native ductus to which the valve is connected is fully cut off from continued flow, flow instead fully diverted, that is, redirected out through side-stem 19 and the bloodline shunt connected to the valve. The valve is then said to be fully closed. By the same token, when the flow diversion chute 18 is fully retracted, flow is completely undiverted, or unaffected, the valve then said to be fully open.

Whether to the bypass or to the native lumen, all valves are always open to some extent, and none are ever ‘closed,’ As can be seen in FIG. 2, when fully retracted, stopper, or obturator 30 closes off and seals the side-stem outlet channel. Depending upon the entry route of the accessory channel 8 into the valve, of which the more common variant is shown in FIGS. 2 and 5, obturator 30 may be securely fastened by rivet or plastic welding, for example, directly to the front of flow diversion chute 18. In FIGS. 7, 8, and 10A, druglines are inserted into the accessory channels to allow the direct pipe-targeting of drugs into the valve and its substrate native vessel.

Channeling of both the upper and lower, or integral, drugline and accessory channel are addressed below in this section. In FIG. 15A, the flow diversion chutes positioned at the half-way point of centrally in their entire throw indicate first, that the valves are continuously variable, therefore servovalves, and that the reciprocally cross-circulating recipient, or native, and donor, or graft, hearts are both receiving and releasing, that is, exchanging, equal volumes of blood. This is reflected in the fact that the flow directional indicator arrows 41 show that rather than for all the blood to be diverted or to flow past the valves, blood is both diverted and passes the flow diversion chutes 18.

That is, an organ transplant almost always of the gradual, or metered, rather than the sudden switch kind, servovalves, continuously variable in moving the flow diversion chute, rather than highly damped nonsparking solenoid-driven valves are used. The appearance of flow directional indicator arrows 41 that continue past the valves mean that the valves are momentarily set to an intervening point in their overall throw, which pertains to metered transplantation and tuned shunts. In FIG. 15A, the straight-line cutoffs on the left-hand donor side indicate that the graft organ has now been severed from the circulatory system of the donor, up to which point blood had continued to circulate through the donor.

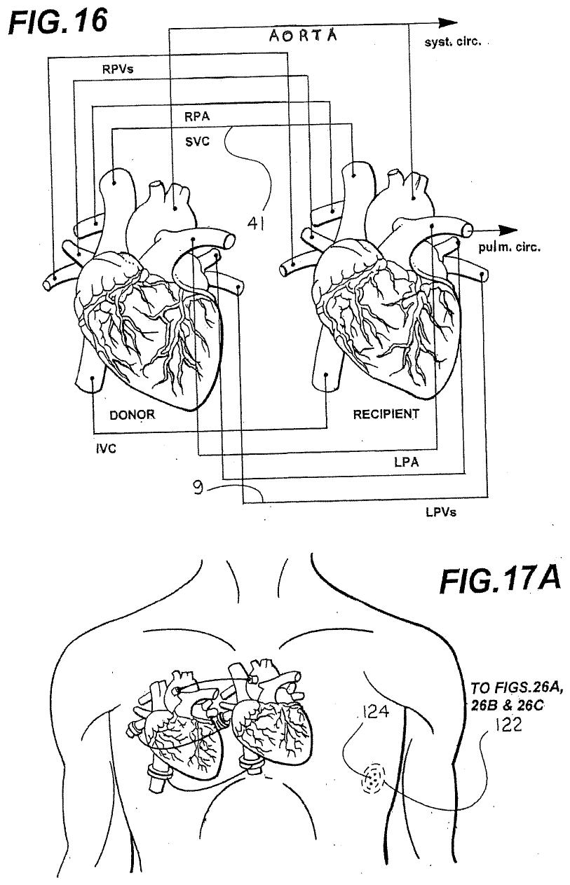

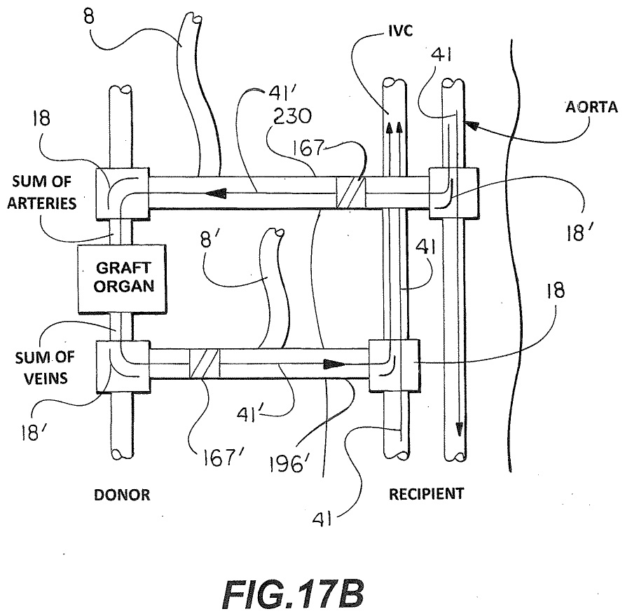

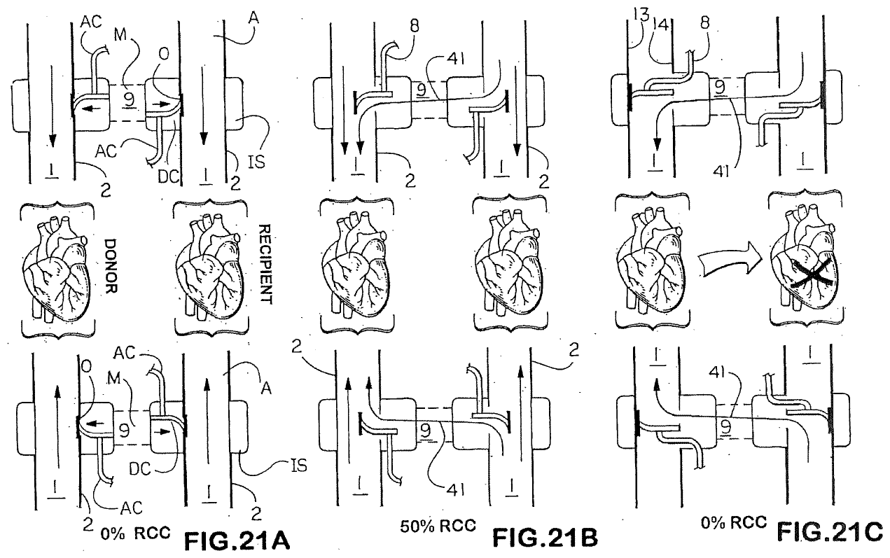

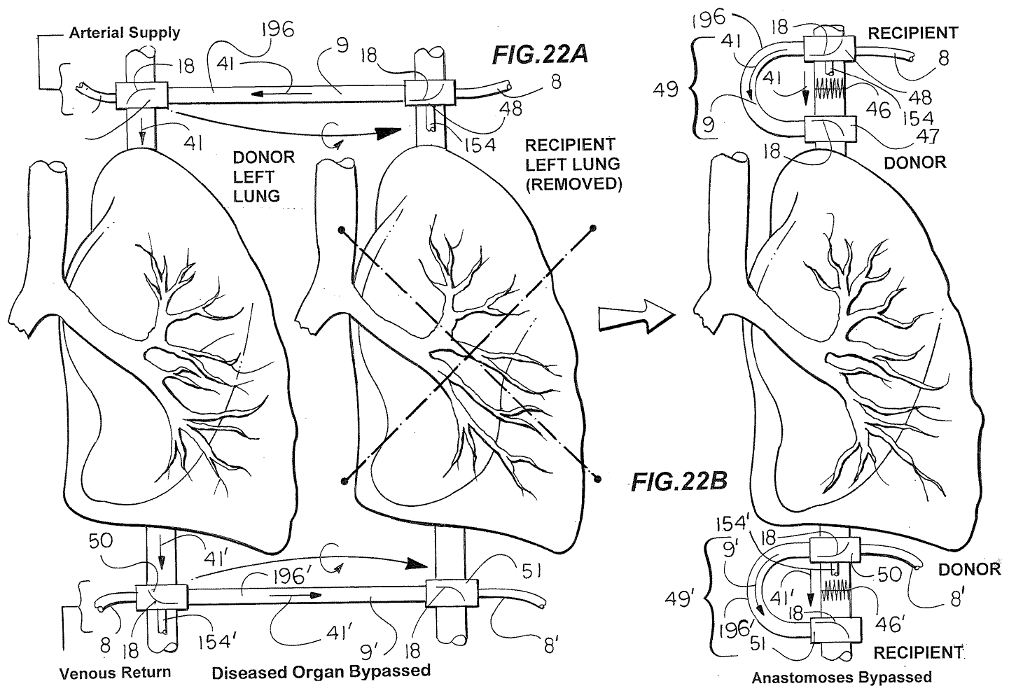

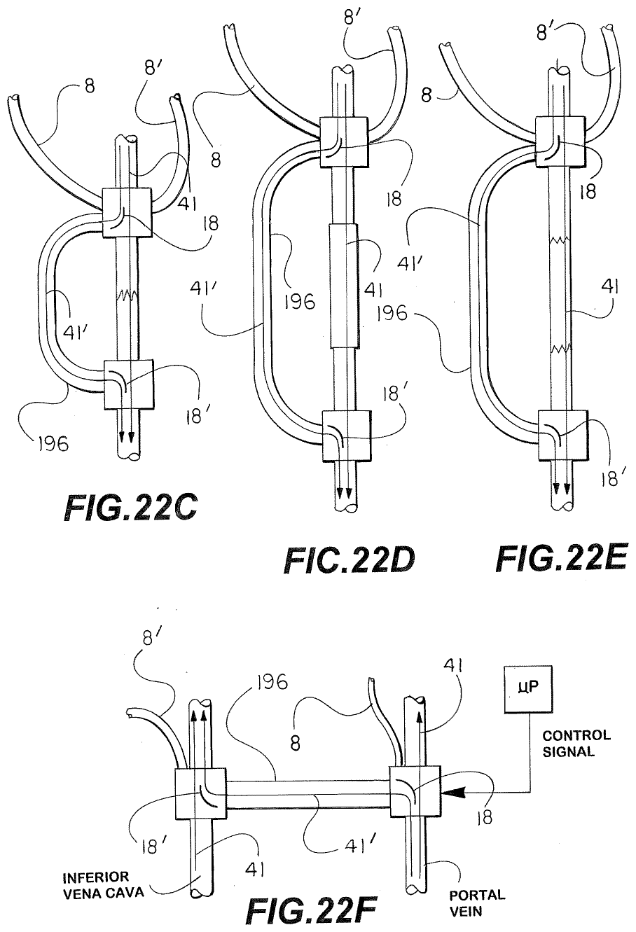

Except for this detail, FIG. 15A represents a temporary, half-way, point in an orthotopic compound vascular bypass, or switched-organ, heart transplant, which the left-hand vessels having been transected is the same as the final position in a heterotopic switched-organ double heart organ transplant. In these figures, the heart is shown as exemplary, any other solid organ transplanted in the same manner. In FIG. 17B (and FIGS. 22C thru 22F), the drugline to the inlet side-entry vascular servovalve is shown as part number 8, while the drugline to the outlet servovalve is shown as part number 8′, and flow that continues through the substrate vessel is shown as part number 41, while that diverted is shown as part number 41′.

In FIG. 17B showing a direct aortocaval connection in the recipient to the graft organ in the donor through confluence conduits that arterial 230 and that venous 196′, described below, the fact that all flow is diverted rather than some diverted and some not as in FIG. 15A means that whether highly damped nonsparking plunger solenoid-driven in a sudden switch, or servovalve-driven in a metered switch transplant, all of the valves have been brought to their fully extended blood-diverting position bringing the procedure to the endpoint where all flow is from the recipient through the graft organ in the donor which then is ready to be harvested for positioning in the circulatory system of the recipient.

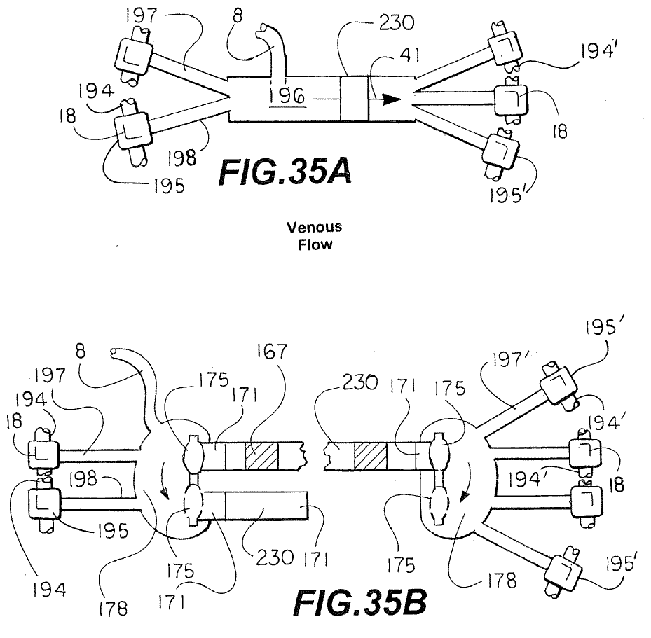

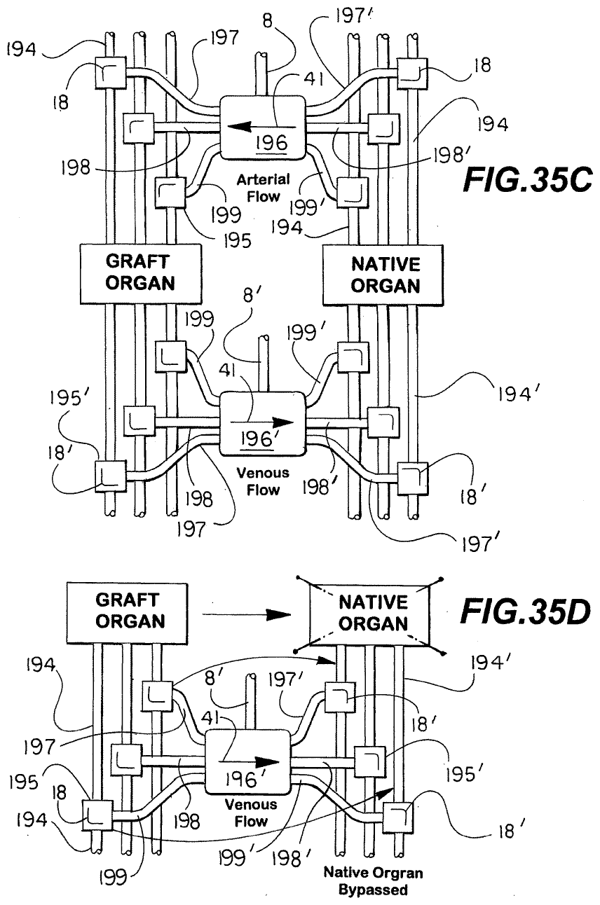

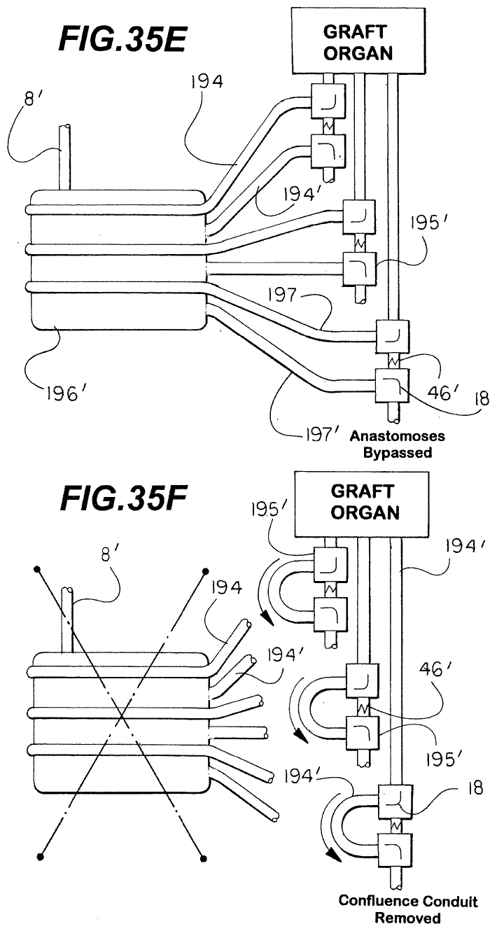

Confluence conduits are described below in this section, a confluence conduit is shown in FIG. 35A, and a switchable confluence conduit in FIG. 35B. In FIGS. 35A and 35B, only the fraction of the blood volume tapped or drawn off from each of the contributing vessels of the donor for continued flow into the confluence conduit are shown, the fraction that continues through the donor vessel omitted. This is equivalent to the entire volume of each passed to the confluence conduit when the servovalve diversion chutes are fully extended.

FIG. 35E shows the penultimate second step in solid organ transplantation for using the bloodlines of a confluence conduit as bypasses around each anastomosis as shown in FIGS. 22B and 35F. FIGS. 35C thru 35F are fully addressed above in section 2b(1)(b)(b15), entitled Switchable Lines and Confluence Conduits: Long When Intercorporeal, to Short When Intracorporeal, Both Affording Connection of One or More Bloodlines to the Same or a Different Number of Bloodlines with Self-leveling Apportionment of Outflow.

In FIG. 17B, moving counterclockwise as does the blood diverted from the recipient on the right to be channeled through the donor graft organ on the left and returned to the recipient on the right having transferred the graft organ into the circulatory system of the recipient functionally but not yet physically,
1. The valve at the upper right on the aorta of the recipient is oriented as shown in the drawing figures of valves with its flow diversion chute fully extended so that all blood flow through it exits out through its side-stem 19
2. The valves on the supply arteries of the graft organ while physically still in the donor summarized at the upper left are oriented upside down in relation to the drawing figures of valves, therefore also reversed in relation to the orientation shown in FIG. 2, so that all blood flow through it enters through its side-stem 19 to be diverted downward.
3. The sending valves on the veins of the graft organ while still physically in the donor summarized at the lower left are oriented in reverse but not upside down in relation to the orientation of the valves shown in the drawing figures of valves with outflow led in the opposite direction out through its side-stem 19, and
4. The valve on the inferior vena cava of the recipient at the lower right is oriented backwards and upside down in relation to the orientation of its sending valves in the lower left corner.

While an organ transplant will more often be metered using servovalves, as shown in FIG. 17B, all of the valves have been synchronously brought to their endpoint bringing their flow diversion chutes to the fully extended position. For that reason, even were servovalves used, earlier flow around the compound vascular bypass circuit is entirely through the valves with no flow directional indicator lines shown passing the flow diversion chutes. This is why flow directional arrow such as shown in FIG. 15A showing the midpoint rather than the endpoint in transfer of the organ from the donor and into the recipient are not seen.

The donor heart having been cut off from the donor, further microchimerization now depends upon continued cross-acclimatization between the donor heart and the recipient over time and the addition as necessary of immune tolerance-inducing medication targeted into the circuit through the valve accessory channels. The latter will have been made evident by hesitation in the advancement of the flow diversion chutes under the control of the system master control microprocessor in response to rejection detecting sensors signaling the need for such medication.

The process of transfer between circulatory systems completed, the defective native organ of the recipient is excised and the donor organ, remaining in the circuit, is removed and positioned in the lower abdomen or in the chest of the recipient according to the type transplant and apparatus appurtenant of those enumerated above in pertinent segments of sections:
2a, entitled Background. General.
2b(1)(a), entitled Sudden Compound Vascular Bypass, or Sudden Switched-organ, Transplantation.
2b(1)(b), entitled Metered Compound Vascular Bypass, or Metered Switched-organ, Transplantation.
2b(1)(b)(b5), entitled Heterotopic Double Heart Metered Compound Vascular Bypass, or Switched-organ, Transplantation and the Use of Unified Entry Arterial and Venous Exit Confluence Conduits to Avoid Disuse Atrophy when the Graft Organ is Positioned in the Lower Abdomen by Direct Aortocaval Connection.
2b(1)(b)(b14), entitled Confluence Conduits: Connection of One or More Bloodlines to Flow through a Common Channel to the Same or a Different Number of Bloodlines with Self-leveling Apportionment of Outflow.
2b(1)(b)(b15), entitled Switchable Lines and Confluence Conduits: Long when Intercorporeal, to Short when Intracorporeal, Both Affording Connection of One or More Bloodlines to the Same of a Different Number of Bloodlines with Self-leveling Apportionment of Outflow.
2b(1)(b)(b16), entitled Direct Aortocaval Connection: Unified Entry Arterial and Exit Venous Confluence Conduits for the Heterotopic Connection of a Graft Heart in the Lower Abdomen.
2b(1)(b)(b17), entitled Doubly Heterotopic Double Heart Compound Vascular Bypass, or Switched-organ Transplantation.
2b(1)(b)(b18). Hybrid Doubly Heterotopic Double Heart Transplantation.

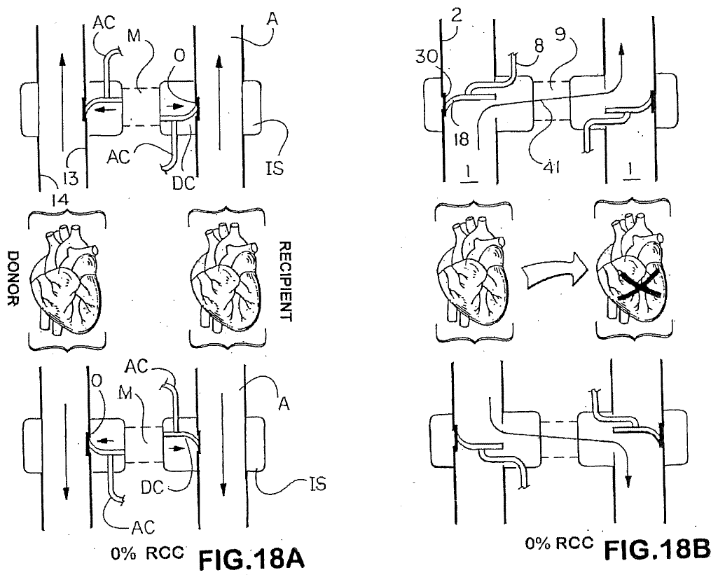

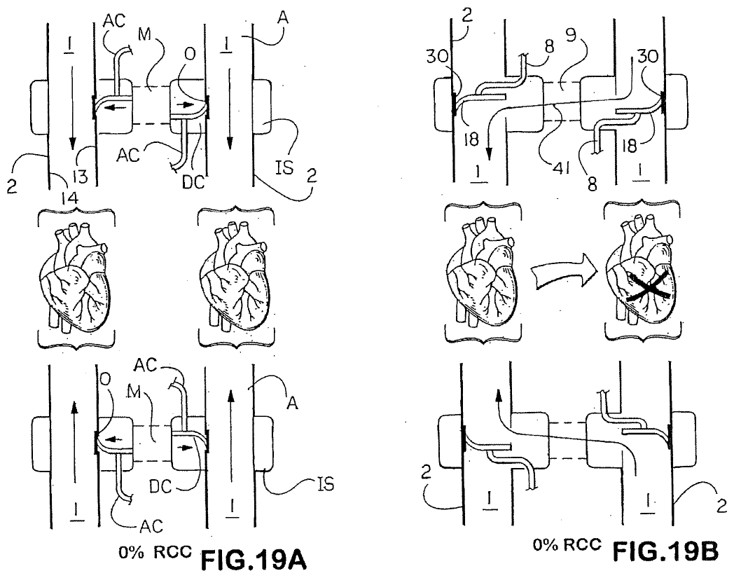

Full closure with full flow diversion is the condition of the valves required at the endpoint of reciprocal cross-circulation between the donor and recipient when the graft organ has been fully transferred from the circulatory system of the donor and into the circulatory system of the recipient, the diseased native organ of the recipient then removed with the graft organ removed from the donor and positioned within the recipient. FIG. 18B depicts this moment in a compound vascular sudden switch orthotopic heart transplant using highly damped nonsparking solenoid plunger driven, hence, with bistable arterial blood flow valves, while FIG. 19B depicts the same moment with respect to the venous valves

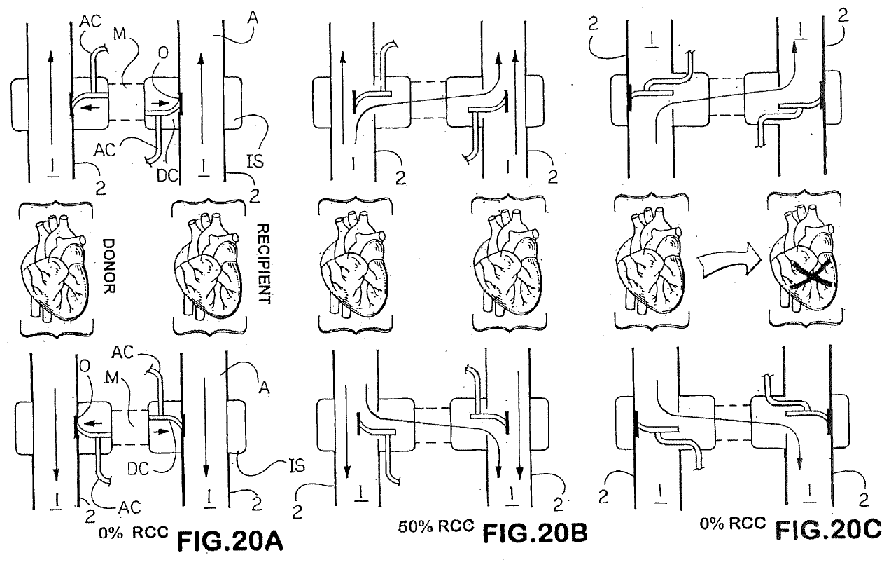

In FIGS. 18B and 19B, the endpoint in arterial and venous flow respectively is depicted for a sudden switch, while in FIGS. 20C and 21C, the endpoint is depicted for a metered switch heart transplant. A transplantation and therefore almost certainly having been performed using servovalves, were the figure showing the condition of flow drawn to represent a moment earlier in the organ transfer process, a small proportion of flow would be shown by flow directional indicator arrows 41 to pass each of the valves.

FIGS. 20A thru 20C depict arterial flow and FIGS. 21A thru 21C the venous flow in such a transplant using continuously adjustable valves, or servovalves, which allow an intervening stage between full containment of the organ within the separate circulatory systems and the endpoint for harvesting and physical transfer of the graft organ. During this intervening interval, the prosthetic disorder response system advances the blood diversion chutes at a rate contingent upon the signaling of rejection by the implanted sensors.

It then halts and/or causes immunosuppressive or immune tolerance-inducing medication, for example, to be directly pipe-targeted through the system druglines and valve accessory channels. This process of cautious, or guarded, advancement is referred to as metered. In FIGS. 22C thru 22F (and FIG. 17B), the drugline to the inlet side-entry vascular servovalve is shown as part number 8, while the drugline to the outlet servovalve is shown as part number 8′, and flow that continues through the substrate vessel is shown as part number 41, while that diverted is shown as part number 41′.

Other circumstances where diversion must be apportionable, or allocatable in a continuously variable manner, include anastomosis-supportive bypasses to allow the apportionment of blood between an anastomosis bypass and the organic lumen with medication targeted in to either pathway, shown in FIGS. 22B thru 22E, and tuned shunts such as that shown in FIG. 22F used to tap, or draw off, a volume of blood sufficient to bring the pressure within a hypertensive source vessel, such as the pulmonary and/or portal hepatic vein, to within the normal range. Accordingly, when such an apportionable device is partially open, flow directional indicator arrows 41 will appear to indicate both the fractions diverted and undiverted, which is the condition depicted in FIG. 22F.

Ordinarily, a tuned shunt such as that shown in FIG. 22F continuously responds to the instantaneous degree of hypertension by adjusting the volume of tapped off blood accordingly, that is, in proportion where the process of adjustment in flow diversion is dynamic and continuous such that the response varies in proportion to the excessive pressure. As explained above in section 2b(2)(h)(h1), entitled The ‘Tuned’ Shunt, in a hierarchical system, treatment is fundamentally improved by not only controlling the degree of diversion or tapping off of blood into another vessel which a monomorbid control system can provide, but through the automatic coordinated dispensing of medication to the etiological and pathophysiological sites associated with the hypertension by a hierarchical control system can come closer to a meaningful suppression of the elevated pressure.

The ensuing reduction in the degree of hypertension then causes the medication if used at all, and therewith, the volume of blood diverted, to decrease or stop. This wide spectrum continuous counteraction does not represent a cure as such, but should progressively minimize if not completely eliminate the hypertension. The distinction between this and a cure is that whereas a cure denotes that the adverse condition disappears, here the condition is controlled only so long as the system is employed.

Hypertension chronic, FIG. 22F shows the tuned shunt in its far more usual state, which is responsive to some degree of hypertension. For that reason, FIG. 22F is depicted as diverting a volume of flow that would otherwise pass through the substrate vessel with the balance undiverted, that is, passed through. Part of the flow diverted and part not, two blood flow directional arrows 41 appear, one showing flow through the concavity of the chutes as partially extended with the balance flowing behind the chute as undiverted. Most often, the volume diverted would represent a relatively small fraction of the total. Less often, the hypertension reduced to zero, zero blood would be diverted so that only one flow direction arrow 41 would appear directly through the substrate vessel behind the diversion chutes which would be fully extended.

In FIG. 22B, the lung transplant will almost certainly have been performed as a metered transplant using servovalves, not as a sudden switch transplant, and the fact that all flow is passing through the anastomotic bypasses indicates that the servovalves have been brought to their fully extended position. However, whereas solenoid-driven valves would allow all flow exclusively through either the bypasses or the native lumen, servovalves allow a relative portion of flow between the two which offers therapeutic advantages.

In FIG. 22C, the ends of the ductus or nerve are shown as directly anastomosed. The gut in an adult male about 26 feet in length, direct anastomosis following the surgical resection or loss of a segment is usually possible without inducing an irrecoverable dysfunction of peristalsis and segmentation, which recover, much less short bowel malabsorption syndrome (see, for example, Cui Y. Zhang C. Zhang H. Zhang X. Tang Y, and 10 others 2024. “Effect Evaluation of Different Preventive Measures for Ileus after Abdominal Operation: A Systematic Review and Network Meta-analysis,” Online, Heliyon (London, England) 10(4): e25412; Emile, S. H., Horesh, N., Garoufalia, Z., Gefen, R., Ray-Offor, E., and Wexner, S. D. 2024. “Strategies to Reduce Ileus after Colorectal Surgery: A Qualitative Umbrella Review of the Collective Evidence,” Surgery (St. Louis, Missouri) 175(2): 280-288; Guillen, B. and Atherton, N. S. 2023. “Short Bowel Syndrome,” Online, Treasure Island, Florida: StatPearls Publishing Co.; Pironi, L. 2023. “Definition, Classification, and Causes of Short Bowel Syndrome,” Online, Nutrition in Clinical Practice (Hoboken, New Jersey) 38 (Supplement 1): S9-S16; Sinz, S., Warschkow, R., Tarantino, I., and Steffen, T. 2023. “Gum Chewing and Coffee Consumption but not Caffeine Intake Improve Bowel Function after Gastrointestinal Surgery: A Systematic Review and Network Meta-analysis,” Online, Journal of Gastrointestinal Surgery (New York, New York) 27(8): 1730-1745; Lakkasani, S., Seth, D., Khokhar, I., Touza, M., and Dacosta, T. J. 2022. “Concise Review on Short Bowel Syndrome: Etiology, Pathophysiology, and Management,” Online, World Journal of Clinical Cases (Pleasanton, California) 10(31): 11273-11282; Keller, D. and Stein, S. L. 2013. Facilitating Return of Bowel Function after Colorectal Surgery: Alvimopan and Gum Chewing,” Online, Clinics in Colon and Rectal Surgery (New York, New York) 26(3): 186-190). Short bowel syndrome is also addressed above in sections 2a, entitled Background of the Invention: General, and 2b(1)(a), entitled Sudden Compound Vascular Bypass, or Sudden Switched-organ Transplantation, as well as given passing mention in others.

FIG. 22E shows the interpositioning of an autologous or tissue engineered length of ductus where that native had to be surgically resected or was avulsed or crushed as the result of trauma to shorten the native ductus so that a direct end-to-end anastomosis was not possible, an eventuality that the length of the small intestine in an adult at well over 20 feet makes unlikely. The need for an allogeneic graft should occur rarely if ever, but should this eventuality result, drugline 8 would be used to directly deliver anti-suppressant along with antimicrobial and anti-inflammatory medication. Required are a solenoid-driven upper vascular valve with drugline, or accessory channel, a length of tubing which can be cut to size, and a lower side-entry diversion jacket.

FIGS. 22C thru 22E show organ transplantation-unrelated anastomotic bypasses available as a distinct product. Whether these are solenoid or servovalve driven depends upon the application. The fact that all flow is through the synthetic bypasses indicates that regardless of type, the valves have been brought to their fully extended position. Bringing the flow diversion chutes of highly damped nonsparking solenoid-driven valves to the opposite end of their throw, that is, fully retracted, would pass flow through the anastomosed organic lumen. Continuously variable, servovalves would allow apportionment of flow between the bypass and the anastomosed segment. Any kind of medication can be released into the blood flowing through.

In FIGS. 22C thru 22E, the upper valve is oriented as shown in the drawing figures of valves with outflow through their side-stems 19, while those in the lower or post-anastomosis position are oriented inverted, or upside-down, with inflow through their side-stems 19. The absence of flow directional indicator arrows 41 in FIGS. 22C thru 22E showing applications for transplantation-unassociated anastomosis bypass devices makes it impossible to distinguish whether the vascular valves used solenoid-driven valves or servovalves with their flow diversion chutes fully extended. However, were the pattern of flow depicted during the preceding moment, the apportionment of flow through and past the valves would have made it evident that these were servovalves.

Even when the servovalves are not set exactly alike, both the volume of flow diverted through the servovalves and the volume passing them is always equal and complementary within the total volume of flow within either stream. A tuned shunt such as shown in FIG. 22F is always made with a servovalve, so that the absence of flow directional indicator arrows 41 that pass by the valves indicate that the flow diversion chutes are fully extended. In FIG. 22F, the sending valve on the right is oriented as are the valves shown in the drawing figures of valves with outflow through its side-stem 19, and the receiving valve at the lower left is oriented in reverse with inflow through its side-stem 19.

Less costly highly damped nonsparking solenoid-driven valves will usually be satisfactory as allowing all flow to be alternated between the anastomosed native lumen and the bypass. Flow through the native lumen can include an antimicrobial, thrombolytic, or anti-inflammatory, for example, added through the valve accessory channel. Were the bypasses themselves organic, the valves with accessory channels would allow these to be medicated through the blood passed therethrough. Flow through the bypass can include any of these or a crystal accretion solvent, for example; however, such an additive would be in the blood passed therethrough.

The implanted microcontroller directing the release of the required substances from different implanted drug reservoirs, where medically justified—although the medicinals and maintenance agents could not be apportioned separately from the blood—a single servovalve would allow an apportionment between the two paths with these different therapeutic and maintenance substances through one and the same accessory channel for the organ and synthetic lumina in alternation. If organic, to separately release medication into the bypass 196 would require a separate drugline to the bypass. If synthetic, a maintenance solvent or other agent would be pipe-targeted into the bypass in the same way.

Other general comments pertaining to vascular valves and servovalves are that for small vessels, the valves are likewise small, even tiny, making the incorporation of means for preventing clogging, even partial obstruction, due to coagulation, biofilm, and along the urinary tract, the accretion of crystal essential. The use of suitable deposition unsusceptible if not repellent materials, coatings, and the institution of an intermittent thrombolytic or crystal solvent drip are addressed above in section 2a, entitled Background. General. The flow-through cross-sections of confluence conduits described above in Section 2a, entitled Background. General, as well as below are actually the sum of those entering and exiting the conduit but shown as larger to allow the bloodlines continuous therewith to be shown more clearly.

Full deployment signaled to the operator by an audible click when detent eminence 16 enters detent depression 17 posing resistance of the chute to further advancement, use of valves such as that shown in FIG. 2 do not depend upon visibility. Sliding of accessory channel 8 with chute 18 is facilitated by collar 12 made of a hard and slippery gear grade nylon. The ostium cut and plug removed, diversion chute 18 is advanced to fully close off native lumen 1 and divert the flow of urine through mainline 9, here a neoureter as shown in FIG. 28. Passage continues through the components of the prosthesis required by the condition of the patient, and eventually into collection bag 149.

Fully extended (deployed, advanced), the typical 2.0 to 4.0 millimeters to span ureteral lumen 1, flow diversion chute 18 is slightly larger than the luminal diameter; so that made of a soft rubbery material and progressively thinned, or feathered, moving toward its periphery, and placed in apposition or abutment against a native surface itself compliant, chute 18 fully seals off the ureteral lumen beneath, or upstream to it, from that retrograde above.

Such a fixed urinary and blood flow diversion jacket, shown in FIG. 2, is suitable for use in a prosthesis where the need for adjustment after placement is not an issue. With the embodiment shown in FIG. 2, an upstream basic side entry jacket can be placed to periodically target medication to the downstream intima as an automatic drip controlled by the implanted microcontroller or microprocessor as might any accessory channel, while water jacket unrelated, or incorporated, accessory channel 8′ allows the diffusion through open cell viscoelastic polyurethane foam 4 of medication to target the downstream adventitia.

Mainline 9, temporarily connected to a vacuum pump to retract the plug of tissue cut from the side of the ductus thereafter serves as the permanent conduit conducting blood or urine out of, or in a shunt or bypass, into the ductus. Devised for application along a ureter rather than a vessel, incorporated accessory channel 8 with lower accessory channel, or drugline, exit pore 31 beneath diversion chute 18 as well as an ostium obturator, or stopper to first prevent extravasation and once introduced into substrate native lumen 1, apportion blood between that tapped off and that to continue along the vessel is optional.

A separate line, smaller in gauge than mainline 9, the sideline 8, is connected to the outlet of a small flat drug reservoir implanted in the pectoral region to serve as the drugline or sideline 8 feeding into the accessory channel, also 8 to make it clear that both the line and accessory channel through the jacket or valve represent the same continuous passageway. Instead of a water jacket, valves for placement along vessels such as those shown in FIGS. 5, 7, and 8 incorporate an ostium obturator 30 which blocks blood from escaping while trepan 6 cuts the plug of tissue to create the side-entry opening, or ostium and once the ostium obturator is brought flush against the near endothelial surface 13 of lumen 1.

In a sudden switch organ transplant driven by highly damped nonsparking plunger solenoids such as those shown in FIGS. 7 and 8, the ostium obturators are abruptly moved through ostium to rest flush against the endothelial lining on the far side 14 of the substrate vessel. In a vascular servovalve, the ostium obturator is initially positioned flush against the near wall endothelium 13 of lumen 1 by the operator through the use of a direct manual fine control mechanism such as that shown in FIGS. 10A thru 10C before electrical control is initiated, whereupon the microcontroller or control microprocessor registers this positional reference datum. Once this position is entered, the balance of the transplantation procedure and attendant administration of drugs as necessary is entrusted to the implanted microcontroller or microprocessor for execution under automatic control.

Adjustable, or controllable, diverters allow switching from nondiverted flow, that is, normal ureteral through-flow into the bladder or diverted flow into a bag, for example. This allows switching between the periodic release of medication into the bladder and wetting down the walls of the neoureters with an antimicrobial or crystal solvent, for example. Both nonadjustable and adjustable diversion jackets requiring the diversion chute to be slid at least forward, the sliding base plate at the bottom of the diversion chute and the sliding track or rail along which it rides, seen in the cross-sections that follow are included in all diversion jackets.

Once the bladder has healed, the neoureters are endoscopically removed and the diversion chutes left in place to allow the continued release of medication directly into the ureters. Then the diversion jackets should be bilateral. Part number 10 is a lock nut to fix trepan tube 5 in position, 11 are spring hinges that urge the jacket into a closed position to stably encircle about to grasp the substrate ureter 12 is the accessory channel stabilizer, 13 is the urothelial lining of the operator-proximal luminal wall, 14 the urothelial lining of the operator-distal luminal wall, 15 suture loop to pass through suture if the ureter with jacket should be held away from neighboring tissue, 16 the sliding positive (male, eminent) detent, and 17 the receiving, or female, detent to retain diversion chute 18 in position unless the operator exerts sufficient force to move it.

Detents are unsuitable for use in servovalves such as shown in FIG. 10A, which are continuously adjustable and were it necessary, can be programmed to hold fast. Only non-servovalves, that is, bistable valves, which are manually set in shunt or bypass prostheses in the urinary tract such as shown in FIG. 2 and highly damped nonsparking solenoid-driven valves such as shown in FIGS. 7 an 8, also suitable for nominally one-time setting of prostheses as irreversible, of which the use may be unavoidable for an emergency sudden bypass, or sudden switched-organ, organ transplant requiring the simultaneous energization of numerous such valves incorporate detents.

If necessary, such detents can be overridden through the application of somewhat greater than usual force. The reason servovalves are preferred for transplantation is the mid- and postprocedural versatility of these vis a vis nominally one time solenoid-driven valves. Servovalves, however, require a prescription-program for the implanted controlling microprocessor adapted for the specific patient, which requires lead-time that an emergency precludes. A solenoid-driven transplant can respond to a prescription-program limited to the direct pipeline targeting of drugs but not valve adjustments responsive to physiological indicia.

Where the use of solenoid-driven valves is unavoidable, greater vigilance of the patient and the manual administration of drugs, for example, is necessary. Restriction to the clinic rather than the automatic release of drugs in an ambulatory patient is in itself a deterrent. Except in a patient considered too frail to undergo revision, solenoid-driven valves should eventually be replaced with servovalves operating under a personalized prescription-program. Awareness of this eventuality is justified only when the need for immediate action does not allow adequate time for preparation.

Indicated by the absence of an ostium obturator, the diversion jacket shown in FIG. 2 is a ureteral or vascular takeoff jacket for placement at the inlet to a shunt or bypass, here a neoureter, where it will remain permanently as a nonadjustable full flow diversion prosthesis. As indicated above in this section, remote controlled adjustability of a takeoff jacket by means of push/pull, or Bowden cable or an electromechanical actuator renders it a valve, which not limited to full flow diversion, must incorporate an ostium obturator to prevent diversion, or outflow when the diversion chute is fully retracted, and assists the diversion chute in splitting the flow when partially extended.

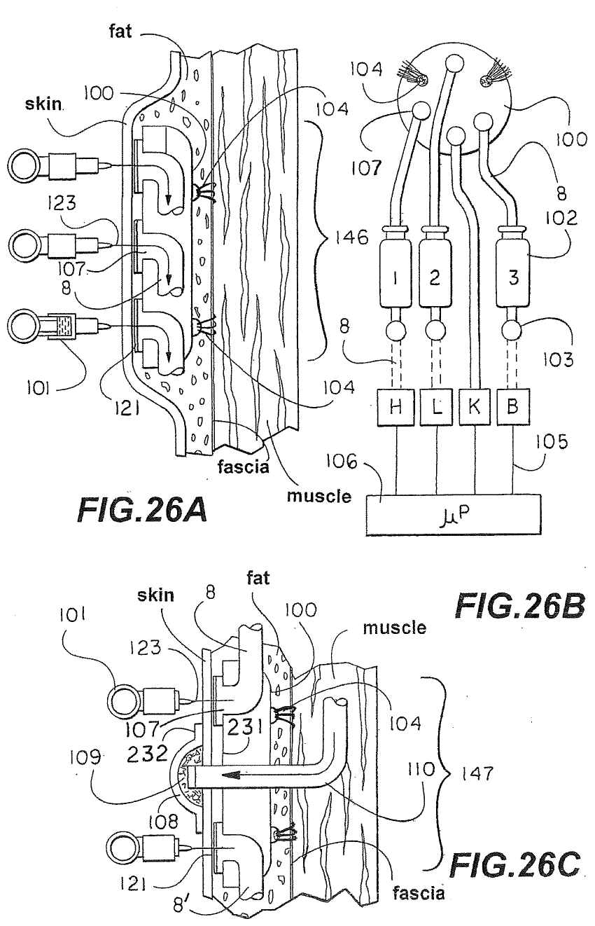

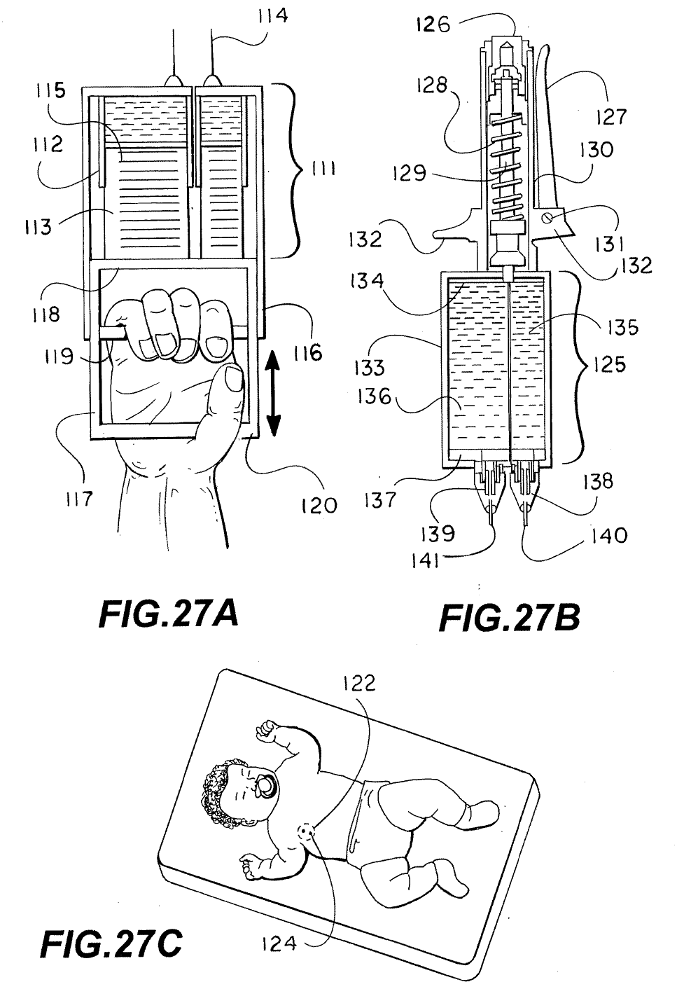

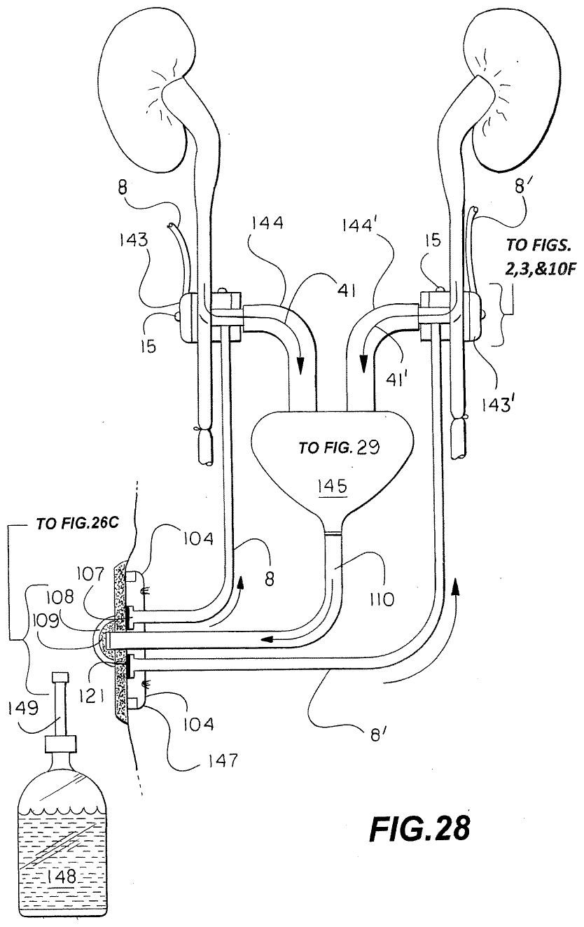

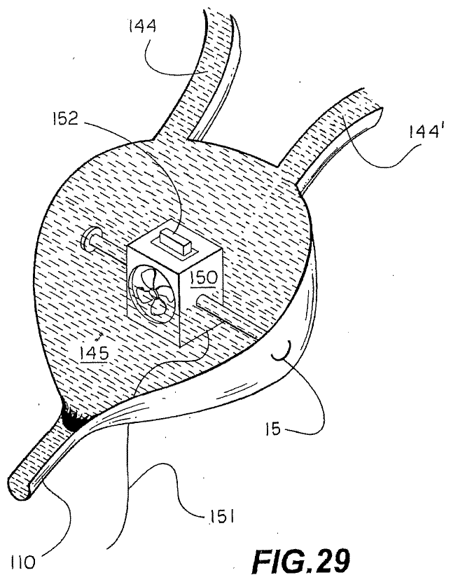

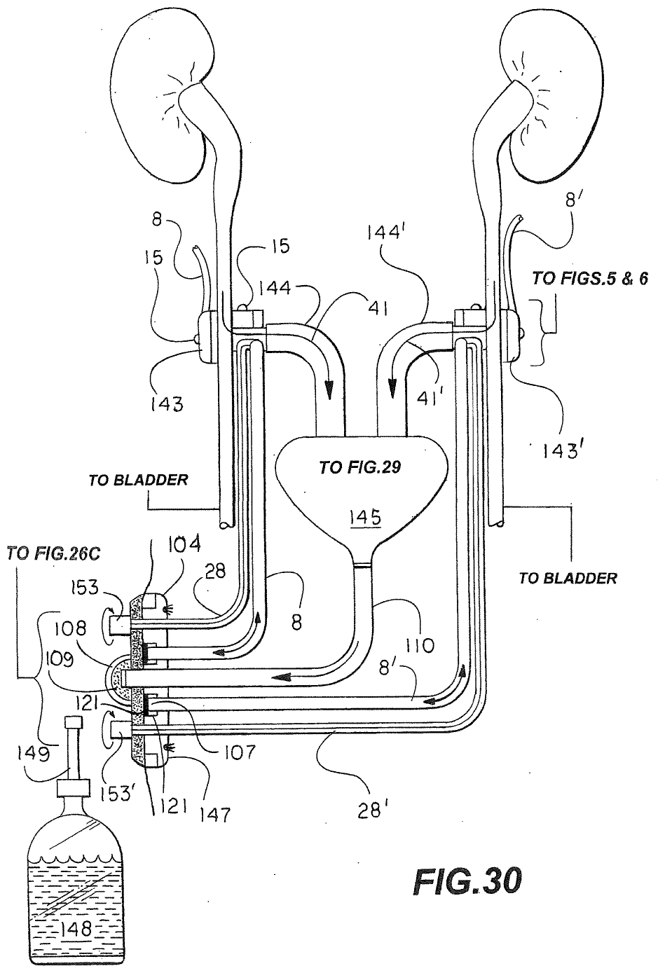

The operator pushes accessory channel stabilization collar 12 forward until an audible click indicates the engagement of positive sliding detent or eminence 16 in stationary receiving detent or depression 17. The detent is not so resistant to disengagement that the operator is unable to retract it should the need arise. The valve shown in FIG. 5 appears as part number 143′ in FIG. 30. A separate larger incision is needed to introduce neobladder 145; however, removal of the native bladder at the same time would necessitate a preexisting open field. In FIG. 30, Bowden cables allow the wearer to adjust the diversion chutes by turning a small dial on a small body surface port with urine discharge opening at the center such as that shown in FIGS. 26C, 28, and 30. When used to aid placement, the cable is disconnected before closing.

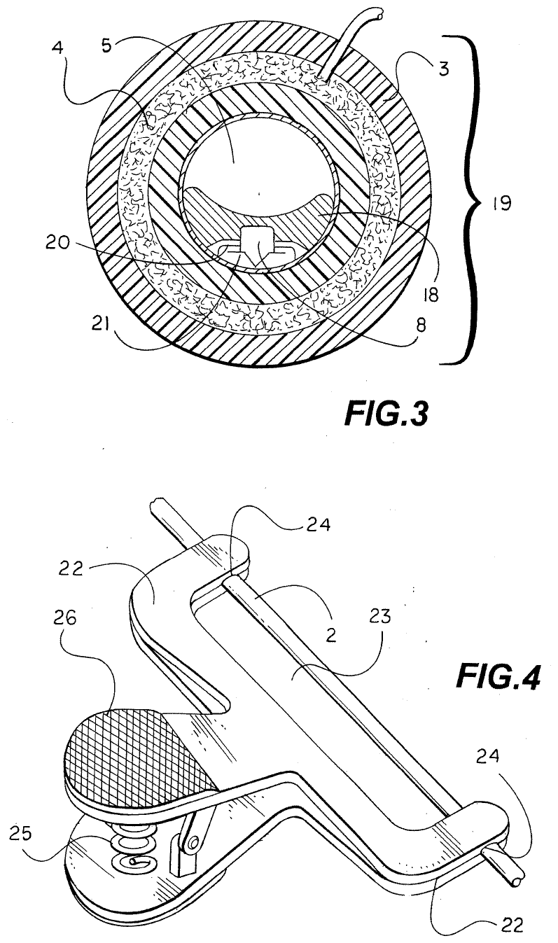

FIG. 3 is a cross-section taken along line A-A′ in FIG. 2 through side-stem 19 of the diversion jacket shown in FIG. 2 to provide greater clarity as to the structure of the accessory channel 8 at the bottom center of diversion flow chute 18, and that of sliding baseplate 20 which rides over slideway, or raceway, track, or rail 21. Accordingly, as shown in FIGS. 3 and 6, the chute 18 with accessory channel 8 travel along a raceway or slideway comprised of horizontal extensions or wings 21 which to assure smooth and properly aligned movement ride within guide restraints. Over the segment of the proximal groove past the tubular accessory channel and extending toward the ureteral lumen, or distally, to the level halfway into the ureteral lumen with the chute deployed, the groove is lined with thin inverted V stock stainless steel with a horizontal side wing to either side impressed by bending the V stock longitudinally along its length with a press brake or draw press.

The wings ride along the bottom of the terminal segment of the mainline with trepan leading edge beneath guides made of slippery gear-grade implantable nylon or polyvinyl chloride comprising an alignment track (raceway, slideway). In addition to smooth and properly aligned advancement of the chute into the ureteral lumen, the stainless steel (inox) liner supports the chute to prevent downward prolapse into the ureter and imparts the stiff relation to assure positive engagement with an audible click of the detents that signals full deployment. Both trepan and chute can be moved back and forth.

FIG. 4 shows a side-entry jacket or valve jacket placement clamp (straddle-clamp, forked vessel segment-straddling spring-loaded double clamp, forked double clamp). Most ductus can be clamped briefly, exceptions being the coronary and carotid arteries, which are never to be clamped however briefly. Unlike the basic side-entry jacket shown in FIG. 1 wherein the path through the lumen of trepan tube is clear of any obstruction that might pose interference to the extraction of the side-entry tissue plug into which trepan tube 5 must be inserted, a valve jacket which incorporates a diversion chute with ostium obturator at its distal tip, or nose, inside trepan tube 5, thus obstructing the trepan tube lumen as the passageway through which the plug must be extracted, necessitates an alternative method for removing the plug to clear the way for insertion of the trepan tube.

An advantage of the straddle-clamp or separate small clamps where that downstream is place first is that this facilitates use of a trepan tube. Elimination of the trepan tube necessitates the use of an ostium obturator greater in pliancy and resiliency than needed in a valve with a trepan tube. A straddle-clamp, that is, a forked ductus segment-straddling spring double clamp or side-entry or valve jacket placement clamp, provides a simplified means for plug extraction past diversion chute 18. The straddle-clamp shown in FIG. 4 is one of a set including clamps permanently positioned at different distances from one another.

Alternatively, a double clamp can be provided such that one handle controls two clamps which can be adjusted in separation and/or the rotational angle of each. Where the segment is small so that little blood would be lost, two separate clamps can be used, allowing placement of the first downstream to increase the pressure in the segment, and that upstream placed immediately afterwards, thus slightly dilating the segment (as well as the upstream artery), making insertion of the trepan tube easier.

A ureteral takeoff jacket or valve can be placed with the aid of a clamp briefly placed upstream to interrupt continued kidney outflow, but never during a ureteral jet; extravasation analogous to that possible with a vessel is not applicable (Baker, S. M. and Middleton, W. D. 1992. “Color Doppler Sonography of Ureteral Jets in Normal Volunteers: Importance of the Relative Specific Gravity of Urine in the Ureter and Bladder,” American Journal of Roentgenology 159(4): 773-775; Jequier, S., Paltiel, H., and Lafortune, M. 1990. “Ureterovesical Jets in Infants and Children: Duplex and Color Doppler US [ultrasound] Studies,” Radiology 175(2): 349-353.

The forked double clamp is intended to suddenly stop the flow of blood through a segment of a vessel for valve placement. When clamped, the blood in the segment between the clamp arms is trapped, maintaining the segment at its filled, or uncollapsed diameter, so that when the valve trepan with suction pump attached to the trepan tube is used to extract the plug, only the small amount of blood that had been trapped runs out (bleeds, extravasates). With the aid of the clamp, to insert the trepan into the side-entry opening, or ostium created, and lock the jacket in place onto the vessel or ureter is accomplished in seconds. The same procedure can be used in placing any basic side-entry jacket where the extraction of the tissue plug might prove problematic. The momentary interruption in blood flow is innocuous.

Blood in the trepan tube is readily washed out with heparin. Such a clamp can be used to expedite the placement of any type jacket about any type ductus. At least one sideline leading to what serves as the water jacket during placement of a basic ductus side-entry jacket and thereafter used as an accessory channel for the delivery of drugs is attached to the side-entry jacket before placement. Differing only in functional distinction at different steps during placement as clarified by the context and structurally continuous, a water jacket, sideline, and accessory channel are all designated by part number 8. A forked double clamp is structurally little different in structure than an office paper pinch clamps; however, the differences are important.

The differences are that the return force must be no greater than is essential to stop flow through the substrate vessel, the contact surfaces on the vascular adventitia must be cushioned using biocompatible materials, and rather than continuous from side to side, such clamps have a central portion removed to create two arms spaced apart by the length of the segment to be bounded. For such use, the clamp is either coated or made of spring stainless steel. Discouraged is a clamp with adjustable spring return force to allow the use of a single clamp with vessels of different diameters; too little compression will prevent neither partial flow nor continuous extravasation, and too great a force can result in intimal injury.

Instead, the clamps are made in different sizes with different spring return forces, thus precluding complications due to human errors in adjusting the return force as might result in intimal injury. Instead, each size clamp exerts a nonadjustable return or clamping force determined by the inherent and worked factors of the spring steel, spring stainless steel, titanium, or polymer used. FIG. 3 shows the simplest of many possible embodiments incorporating torsion, tension, or compression springs and made of metal or plastic.

In FIG. 4, parts 22 are the clamping arms to either side of straddle-clamp, 23 the space separating clamp arms 22, which defines the segment between clamp arms 22 for the entrapment of blood inside the segment to facilitate the removal of a side-entry tissue plug from the wall of the vessel to be jacketed and placement of the diversion valve. Parts numbered 24 are the adventitial contact area cushions, 25 the extension spring-backed hinge that provides the clamp spring retum force, and 26 the thumb and index finger pads the operator presses together to place, or set, and remove, or release, the clamp. Provided it is coated, the suction pump hose as temporary mainline 9 can be used to pull through the plug, and if successful, can then be detached for replacement with a catheteric mainline through which blood will outflow and exit or drugs enter and inflow.

Otherwise, the suction hose removed, the straddle-clamp is left in place just long enough to allow the use of a small dental pick or similar hand tool to extract the plug. The permanent catheteric mainline through which blood will flow out or a drug flow through can then be attached and the clamp removed. A stopper affixed to the nose of the diversion chute, or ostium obturator, which serves to prevent outflow through the trepan tube when diversion is not in use, appears in the diversion valves and servovalves to be described.

The ostium obturator then prevents outflow long enough for the opposite end of the mainline to be attached. The absence of an ostium obturator indicates that outflow during placement and thereafter is not a problem. The same use of straddle-clamps can be applied to the placement of any basic side-entry jacket where the extraction of the tissue plug might encounter interference, the brief interruption in blood flow innocuous.

FIG. 5 shows a manually operated urinary flow diversion valve wherein flow diversion chute 18 is driven by a push/pull control cable between the fully closed chute extended full flow diversion and fully open chute retracted normal flow through positions. The fully extended position is suitable for use during the night to avoid being awakened by the need to void, whereas the fully retracted position is suitable for use during the day to void normally. Other applications include preventing an interruption during a public performance to the extent that urge sensation is never felt.

With the embodiment shown in FIG. 5, an upstream basic side entry jacket can be placed to supplement incorporated accessory channel 8 and 8′ in periodically targeting medication to the downstream intima as an automatic drip controlled by the implanted microcontroller or microprocessor. Incorporated accessory channel 8′ allows the diffusion through open cell viscoelastic polyurethane foam 4 of medication to target the adventitia. Incorporated accessory channel 8 is available to drip medication downstream of diversion chute 18.

When accessory channel 8′ outlets in front of collapsed diversion chute 18, diversion chute 18 sweeps the medication over the small ostial trepan wound and delivers medication into substrate native lumen 1 when ostium obturator is not set flush against the proximal intima 13. When accessory channel 8′ outlets into the open cell polyurethane protective foam 4, medication continues to diffuse through foam 4 to medicate the adventitia, the extent to which the medication reaches circumferentially determined by the frequency and volume of the drip.

In any adjustable embodiment such as this, an ostium obturator 30, shown here fully extended for full diversion, is essential to prevent outflow through trepan tube 5 when diversion chute 18 is retracted to allow normal ureter-to-bladder drainage. In FIGS. 5, 7, and 8, the ostium obturator, effectively a tiny version of an especially elastic and compliant rubbery polymeric tub drain stopper, is shown both fully extended and ‘ghosted’ to show it prior to deployment when it is retracted in the side-stem outside lumen wall 2. Accessory channel 8 for the delivery of drugs into the substrate ureter to flow thence into the bladder outlets at diversion chute 18 nose underside just within the ostium obturator 30 through opening 31.

A jacket of this type can be provided with a diversion chute detent-fixed in position with or without an accessory channel as shown in FIG. 1 or can be switched between chute 18 fully open or fully closed positions. When switchable thus, the diversion jacket is a valve capable of continuously adjustable control but degrees of extension between fully open and closed are not pertinent for the wearer who usually wishes full diversion during the night and when performing before an audience. However, this does not preclude the use of a mid-way position to delay voiding either normal or through the neobladder shown below. Accordingly, such a manually controlled valve is unsuited for use along the vascular tree where an implanted micro master controller can continuously apply fine adjustment to the valve in response to sensor data continuously fed to the controller.

FIG. 6 shows a cross-sectional view of the diversion jacket shown in FIG. 5 along line B-B′ to show diversion chute 18 handle or accessory channel 8 and diversion chute 18 reciprocal adjustment clearance slot 27 through which diversion chute 18 passes when driven by push/pull cable 28, cable 28 connected to stabilization collar 12 by coupling block 29. The rail device upon which chute 18 rides when advanced into or retracted from the substrate native lumen 1 is the same as that shown in FIG. 3 with the difference, however, that section line A-A′ indicated in FIG. 2 is not through the part of the valve where slot 27 must be cut out for passage of the accessory channel 8 as is the case along section line B-B′ in FIG. 6

To eliminate the need for direct manual contact of the operator with stabilization collar 12 and thus expedite endoscopic placement of a nonadjustable urine flow diversion jacket with optional but usually preferred accessory channel 8 for the direct delivery of drugs into the ureter 1 and bladder, cable 28 can be adjusted by means of a mechanical foot pedal control similar to that on a lawn tractor, then-retraction of chute 18 in this static prosthesis thereafter unnecessary-removed.

FIG. 7 shows a diversion jacket with flow diversion chute 18 deployed by a highly damped nonsparking miniature plunger solenoid. This allows the deployment of multiple chutes at the same instant by pressing a single switch, as is necessary in performing a sudden switch heart transplantation, for example. The device is intended to shift diversion chute 18 suddenly to the fully extended position, there to be left in place. Bistable, that is fully retracted or extended but unable to be positioned at a point intervening between these, the valve is actually less adjustable than is one cable driven, but allows remote control over multiple such devices, essential in a sudden switch solid organ transplant, for example.

With the embodiment shown in FIG. 7, as with any jacket-integral, or incorporated accessory channel, an upstream basic side entry jacket can be placed to periodically target medication to the downstream intima as an automatic drip controlled by the implanted microcontroller or microprocessor. Incorporated accessory channel 8′ allows the diffusion through open cell viscoelastic polyurethane foam 4 of medication to target the adventitia. When incorporated accessory channel 8′ outlets into the open cell polyurethane protective foam 4, medication continues to diffuse through foam 4 to medicate the adventitia, the extent to which the medication reaches circumferentially determined by the frequency of the drip.

When incorporated accessory channel 8′ outlets just ahead of ostium obturator 30 while ostium obturator 30 remains collapsed at the distal tip of diversion chute 30 prior to being deployed, ostium obturator 30 sweeps the medication, typically an anti-inflammatory, over the edges of the small ostial wound just cut. Where accessory channel 8′ with outlet into foam 4 remains effective indefinitely, positioning accessory channel 8′ to outlet just ahead of ostium obturator 30 prior to its deployment results in a reduction in its effectiveness at delivering medication into lumen 1. At all times, incorporated accessory channel 8 remains available to release medication through lower accessory channel, or drugline, outlet pore 31 into the downstream flow.

Simultaneous actuation is by the operator or an assistant by direct wire connection, the wires thereafter removed and the jacket with accessory channel or channels left in place. In certain locations, the anatomy will better accommodate a valve of this configuration rather than that shorter shown in FIG. 8. If needed, suture loops 15 at the abaxial or butt end of the valve side stem allow suture to be run through for connection to nearby connective tissue to aid in defraying the weight of the valve. Unneeded suture loops 15 are easily trimmed away with a scalpel or scissors.

Bistable control thus is distinct from control gained with a servomotor-driven diversion valve such as those shown below, wherewith continuously and valve to valve variable control is exerted by an implanted microcontroller fed pertinent data from implanted sensors and applied by the controller prescription-program. In a patient requiring the simultaneous surveillance and treatment of comorbidities, an implanted microprocessor serves as the master controller over a hierarchical control system programmed to seek out and apply that multimodal therapeutic response most likely to achieve the optimal homeostatic result across all comorbidities during and following a transplantation procedure, for example. The control of comorbidities can critically affect the sustainability of an organ transplant.

More complex control thus requires multiple command-execution channels, which unless posing a risk of strangulating tissue, are delivered along implanted wires. Otherwise, wireless such as radio remote control is used, each actuator singled out through the use of a unique carrier frequency. Part numbers in FIG. 7 are the same as in FIG. 5 with highly damped miniature nonsparking plunger solenoid plunger solenoid 32 replacing the cable drive, 33 the connector of the solenoid plunger (thrust rod, slider, shaft) to accessory channel 8, 34 the solenoid electromagnetic coil or winding, 35 the solenoid compression spring, and 36 the solenoid plunger.

Also in FIG. 7, parts numbered 40 are apertures, which can be slits, or round holes, for example, to allow contact with the outer body cavity of the adventitia, such communication long established as necessary to prevent atherosclerotic degeneration at the intima due to compression of the nervelets, or vasa nervorum, and fine blood vessels, or vasa vasorum of the adventitia. Such perforations or apertures are numerous, most off the plane of section depicted in the drawings.

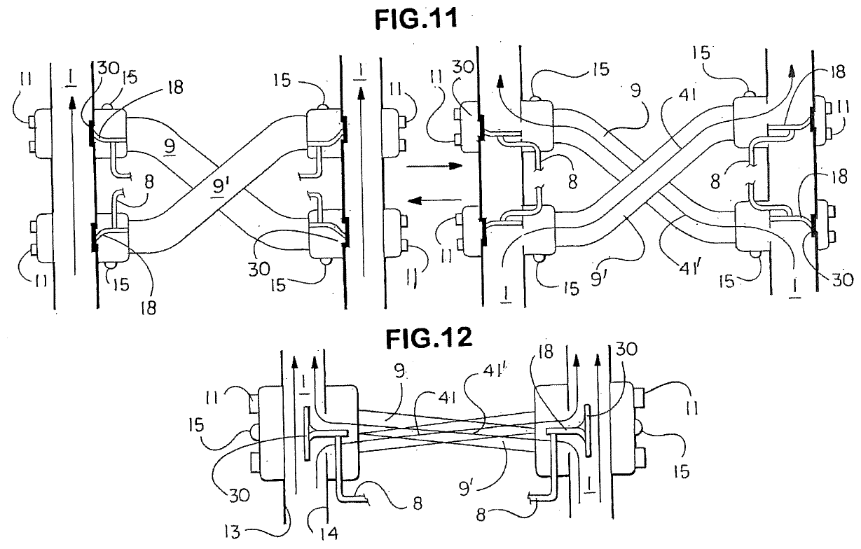

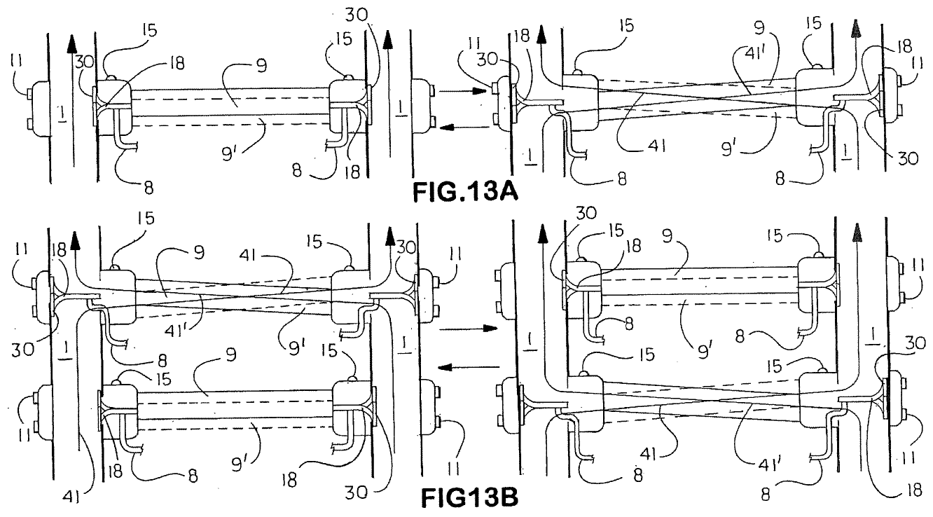

In FIG. 8, highly damped miniature nonsparking plunger solenoid 32 has been positioned in reverse beneath rather than in front of accessory channel feedline 8 beneath side-stem 19 to pull rather than push accessory channel feedline 8 used as the diversion chute handle. Part numbers in FIG. 8 are consistent with those in the other drawing figures. This arrangement allows the valve to be shorter expediting miniaturization for use in an infant, for example. In addition to the use of silver wire in the windings and the use of lightweight plastics, a significant reduction in size and weight for use in a small child is the use of servovalves which incorporate 2-way or bidirectional diversion chutes such as shown in FIGS. 11 and 12.

Accessory channel 8′ allows an automatically released drip to diffuse through the open cell viscoelastic polyurethane adventitia protective foam 4 to wet the adventitia. Were accessory channel 8′ continued through servovalve jacket shell 3 to outlet into the trepan tube 5 just in front of ostium obturator 30 before ostium obturator 30 is advanced into substrate ductus lumen 2, the medication released would coat the ostium about to be cut by trepan 6 at the front end of trepan tube 5 but would then become ineffective for further treatment of the intima. However, both positionings can be incorporated into the valve jacket, just as both can be incorporated into the ductus side-entry servovalves shown in FIGS. 5, 7, 9, and 10.

FIG. 9 shows one of several servomotor types suitable for use in a vascular servovalve wherein it is positioned within the valve jacket as are the highly damped nonsparking plunger solenoids shown in FIGS. 7 and 8. The criteria for a linear servoactuator include precision performance, and suitability for miniaturization to achieve a size and weight least likely to cause discomfort. Unacceptable is any motor design that significantly deviates from a cylindrical form factor, such as a motor mounted to extend away from the central or long axis.

Linear actuators include those which combine a rotary motor with planetary gearing and a machine screw drive to convert rotary to linear motion and those intrinsically linear. Of the two, intrinsically linear designs are direct-drive, eliminating cogging and backlash, and better lend themselves to a tighter cylindricality at the driver end, small size, and low weight. Of the various types of linear motors-iron core, U-channel, tubular linear, miniaturized versions of linear induction machines-one that lends itself to miniaturization and a compact cylindrical shape and provides the level of precise control suitable for use in vascular valves is the pulse tubular linear shaft motor.

Such a motor is brushless, of high flux density with a conformation that affords maximum magnetic efficiency, zero cogging, highly reliable, and precise, as well as direct drive. Void of mechanical linkages, hence, backlash-free, the motor requires no maintenance, is virtually instantaneous in response, and from the standpoint of compactness, is scarcely deviant from a straight tubular shape, making it suitable simple to adapt for use in infants. As shown for plunger solenoid diversion jackets in FIGS. 7 and 8 respectively, the linear or other form of servomotor can be positioned within the valve with connector 33 to push or pull accessory channel 8, without the limitation in performance of full displacement from one end of the stroke or the other.

In FIG. 9, a pulse linear shaft motor of the kind made by numerous manufacturers to include Nippon Pulse Motor Company, Bunkyo City, Tokyo, Japan, comprises a forcer, here adapted with cylindrical housing 37, enclosing adjacent circular electromagnet coils 38 in circumferential and surrounding relation to the motor shaft 39 which incorporates successive pairs of cylindrical neodymium iron boron permanent magnets 42.

The magnets are arranged along the shaft so that successive pairs consist of a forward magnet with south pole at the leading end, for example, and north pole at the trailing end, the following magnet in the pair reversing the poles so that each pair has facing north poles where the members of the pair come together and adjacent south poles where the successive pairs come together. A central requirement in servovalve motors for use in the arterial tree is that the holding torque rating or placement in a closed loop system be adequate to hold fast and not be overcome by the pulse.

Numerous manufacturers make or could make miniature and subminiature linear servomotors to meet the requirements for ductus valve servovalves for use in the vascular tree and urinary tract, for example. These include Aerotech Inc., Pittsburgh, Pennsylvania; Macton Dynamics, Croydon, Pennsylvania; Monolithic Motions Solutions, Kirkland, Washington; HIWIN [HI-tech WINer] Corporation, Taichung, Taiwan; Physik Instrumente, Karlsruhe, Germany; Dunkermotoren GmBH, Bonndorf, Germany, Ingenia, Barcelona, Spain; Magnetic Innovations, Veldhoven, Netherlands; Beijing Inspire-Robots Technology Company, Shijingshan, Beijing, China; Nippon Pulse Motor Company, Bunkyo City, Tokyo, Japan; Orienal Motor Kabushiki Kaisha, Ueno, Taito, Japan; Dr. Johannes Haidenhain, Trauenreut, Germany; and many others.

One alternative type of precision feedback miniature servovalve suitable for implantation is driven by a servomotor which rotates a lead screw, the diversion chute then mounted on a platform riding the lead nut which the lead screw drives linearly in either direction as seen in linear positioning stages and lathes, for example. Such an alternative type servovalve is used if the direct drive type servomotor shown in FIG. 9 allows the diversion chute to displace under elevated pressure as when the native heart pending replacement is overdriven with inotropes. The displacement counteractive capability of a vascular servovalve is established through testing in a surgical research laboratory.

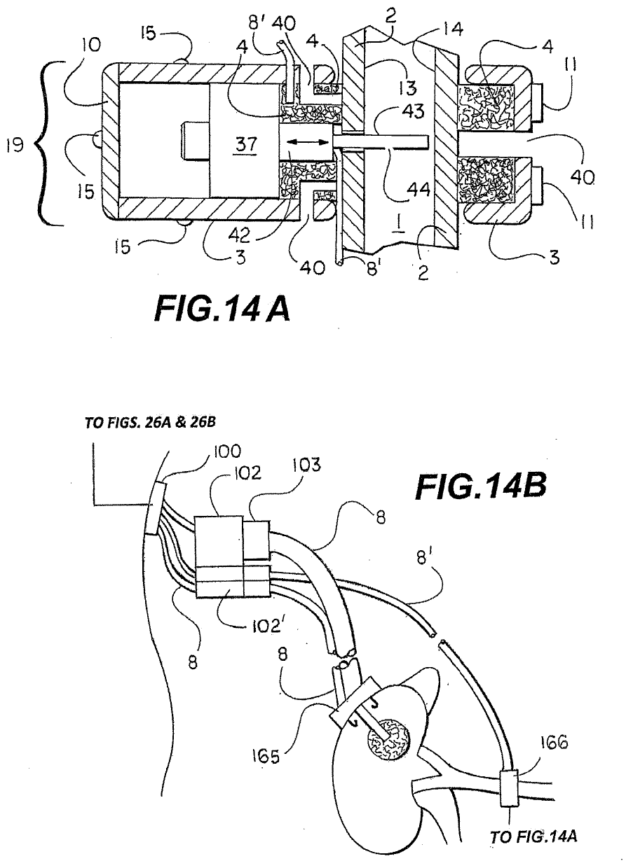

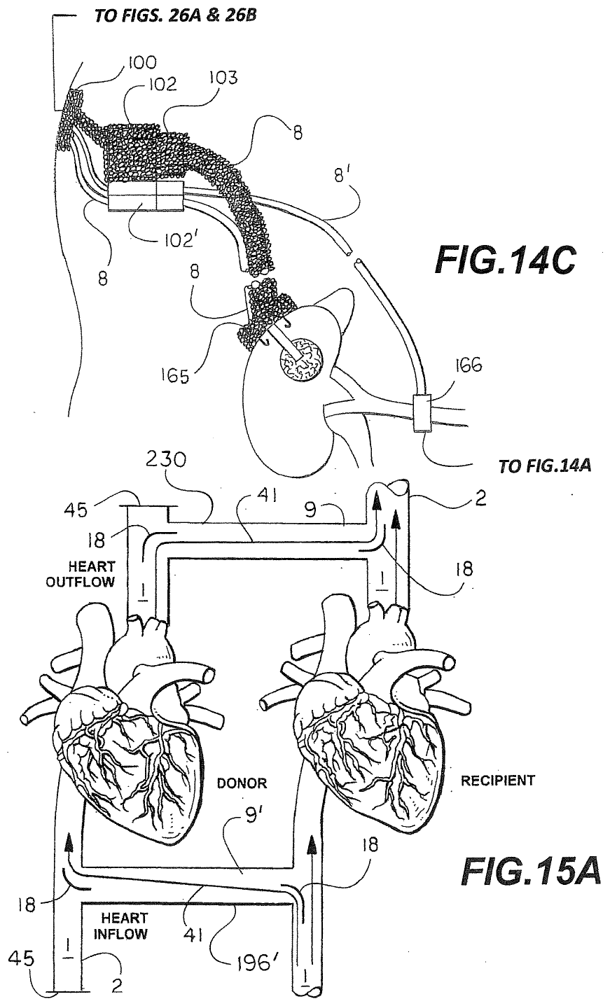

Shown in FIG. 10 depicting a vascular servovalve with direct manual fine control attachment and FIG. 14 of a servochoke, miniaturized versions of such a tubular linear shaft pulse servomotor are suitable for use in any side-entry valve or servochoke. At the sizes required, most tiny, the neodymium magnets should not present an absolute sum weight such that the normal alignment provided by the jacket with foam lining, and if necessary, the aid of suture run through a suture loop 15 and wound about the substrate ductus upstream will exert a levering force to compress or flex the substrate ductus. Accessory channels in the vascular servovalve shown in FIG. 10 can include any or all of those described in conjunction with the valves shown in FIGS. 2, 5, 7, and 8 bearing part number 8 or 8′.

FIGS. 10A thru 10E provide in FIG. 10A a longitudinal midsection through a vascular servovalve driven by a pulse tubular linear servomotor of the kind shown in FIG. 9 with the addition of a direct manual fine adjustment feature which allows the operator to apply minor adjustments in retraction or extension of the diversion chute with ostium obturator into the substrate native lumen, while FIGS. 10B and 10C illustrate how the one control knob allows separate control over engagement of the direct manual fine adjustment device as required in a carotid endarterectomy bypass device, FIGS. 10A and 25A and 25B showing such a vascular servovalve with divided outlet, FIG. 10D providing a more detailed cross-sectional view of the mechanism shown in FIGS. 10B and 10C, and FIG. 10E providing a more detailed view of the miniature endoscopic-type grasper used to stabilize the piece used to connect the detachable manual fine control device to the servomotor housing before and after the servovalve is placed along the substrate vessel.

In placing a valve along a carotid or coronary, it is important that no significant extravasation or interruption in the flow of blood occurs. Accordingly, the use, however momentary, of a clamp, one such shown in FIG. 4 is disallowed. In entering such an artery, first the suction pump applied to the mainline is used to pull the wall of the artery past the trepan to cut a plug of tissue. The pump is reversible and equipped with a limit switch, so that the instant resistance to the vacuum disappears, the pump shuts off. The plug may have adhered within the circle incised, but more likely has been extracted, but blocked by the diversion chute and the undeployed, therefore collapsed, ostium obturator, has become lodged at the front of the trepan tube. The plug must be removed with no interruption in the flow of blood as could induce a cerebral or myocardial infarction.

At this time, 1. The plug must be removed, and 2. Once the plug has been removed and the diversion chute passed into the substrate ostium, the ostium obturator must fit flush over the ostium to prevent blood from leaking.

Removal of the plug is by drawing the trepan tube from the ostium where the operator has wetted his glove with a heparin and positioned the thumb adjacent to the opening. The instant the trepan is removed, the operator slides a thumb over the opening, preventing blood from escaping and an assistant reverses the vacuum pump to expel one or more forceful discharges of air, driving the plug out the front of the trepan tube. If as improbable, following one or more strong puffs, or ‘blasts,’ of air the plug still adheres at the front of the trepan tube, an assistant quickly retrieves it with a tweezers or dental pick. The trepan tube is reintroduced into the opening simultaneously with the sliding away of the thumb and the valve jacket allowed to close about the artery.

A relatively simple way to place a bistable fully open/fully closed highly damped nonsparking solenoid-driven valve such as those shown in FIGS. 7 and 8 or one driven by a continuously variable servomotor such as that shown in FIG. 10A is by withdrawing the tissue plug with the aid of a small suction wand, or suction pipe—a length of transparent tubing the same in diameter as the valve trepan with a ferrule at the distal end having a razor sharp front edge, the tube connected to a suction pump with an on/off switch controlled by an assistant. The sharp front edge is placed against the side of the vessel and suction is applied to draw out the tissue plug. The instant the plug is seen to have entered into the suction wand, the operator withdraws the want and places his thumb over the opening to minimize the seepage of blood.

As the valve is about to be inserted into the opening, the operator removes his thumb aside to prevent any further seepage. The front portion of side-entry valves opens as a jacket consisting of two half cylindrical sides hinged at one side and provided with a pawl and pawl receiving depression into which the tip of the pawl clicks when engaged (not seen as outside the plane of the drawing figures), the forward ductus encircling portion of the valve thus forming a collar to grasp about the substrate ductus. Long enough to hold all of the plugs to be extracted without the need to set aside one and reach for another, the suction wand is set aside until after the operation when the suction is continued to remove the plugs.

Because the walls of larger arteries incorporate elastic laminae and smooth muscle, and because the ostium obturator must comprehend elasticity and resilience consistent with the need to fully deploy from a collapsed condition, despite the use of the vacuum pump or controller in an attempt to retract the obturator into a flush fit against the inside of the side-entry opening, or ostium, some rebound will usually occur which only precise manual control can overcome. Shown in FIGS. 10A thru 10E and 25 is direct manual fine control attachment device to a vascular servovalve. The attachment is used only during attachment of the valve and removed before closing. It is then sterilized by autoclave or ethylene oxide gas for use with another valve of the same type and size.

Attachment of the direct manual fine control to the servovalve is accomplished preoperatively and does not add to the duration of the operation. Adjustment of the ostium obturator 30 to fit flush against the ostium can only be accomplished by the operator mid-procedurally once the diversion chute has been moved into the lumen of the substrate native vessel. Attachment of the direct manual fine control device can serve as a precaution should a sudden vacuum force exerted on the ostium obturation by the suction pump or the automatic program itself not seat the obturator flush against the ostium. FIG. 25A illustrates the prefinal step in placement in a bypass to both the internal and external carotids, and FIG. 25B the same where only the internal carotid must be bypassed just before the attachments, each matched in size to its valve, are removed.

To minimize the weight of the servomotor, coil 38 is wound with silver wire. To this end, the longitudinal extension of the valve and consequent levering moments of force, is kept as small as possible. The pulling rather than pushing driver configuration shown in FIG. 8 is shown adapted in FIG. 10 to allow shortening the valve, reducing its moments of force, and therewith any tendency to lever under gravity, imposing a bending force on the substrate vessel.

FIG. 10A shows a puller-configured double outlet tubular pulse linear shaft motor-driven vascular side-entry diversion servovalve of the kind shown in FIG. 9 with the addition of a detachable direct manual fine control device positioned beneath servovalve side-stem 19 shown in FIGS. 10A thru 10E suitable for performing a carotid endarterectomy, for example, where one valve on the common carotid bypasses blood into the internal of the internal and external carotids. As shown in FIG. 25B, where the external carotid is unaffected, only a bypass to the internal carotid is needed. In both, the diversion chute of the valve on the common carotid must remain partially retracted to allow blood to pass through the common carotid into the facial, lingual, and superior thyroid arteries, as well as numerous arterial ‘twigs.’

Attached to the servomotor housing before use and detached after, the manual fine control, or override, device is used to manually initialize the precise horizontal position of the servomotor shaft if necessary before valve placement along the substrate vessel. The manual fine control device is housed in a separate enclosure which is attached to the motor housing during use and thereafter removed. The direct manual control device assures the correct alignment of the motor shaft for its attachment and allows the operator to apply small adjustments in the position of the servomotor shaft, hence, correct starting position of the ostium obturator to assure a flush nonleaking fit over the ostium.

The double outlet implements use of the valve in a carotid endarterectomy bypass such as that shown in FIG. 25A, which can be used intraoperatively or, where the carotids are heavily diseased of must be resected to remove tumors, as a permanent prosthesis. The direct manual fine control device allows the operator to move motor shaft 39 without snap-back during a condition of no current flow. That is, the direct manual fine control device allows the operator to apply small adjustments in extension or retraction into the substrate native lumen 1 of the ostium obturator at the distal tip of diversion chute to fit flush against the ostium when the implanted control microcontroller or microprocessor has not been additionally programmed to respond to a handheld remote control to allow off-line tiny shifts of the diversion chute and the deployed diversion chute 30 is not flush against proximal intima 13 to prevent any blood from escaping.

### Overall Structure of the Direct Manual Fine Control Device

Part numbers not unique to the direct manual fine control device shown in FIG. 10A are consistent with those shown in the preceding figures. The device enclosure is made of a tough clear transparent polymer such as polyethylene terephthalate, the internal parts rendered radiopaque with contrast, such as Danfoss Tantalum Technologies Danfoss Coating®, thus allowing the operator to directly observe its internal operation whether the valve is applied to the substrate native lumen with side-stem 19 directed to the left as depicted in FIG. 10A or to the right.

To fully deploy, that is, expand from its collapsed position while remaining within trepan tube 5, ostium stopper, or obturator, 30 must enter substrate native lumen 1 to a point slightly beyond a fully flush relation, possibly allowing a slight emission of blood into trepan tube 5. Trepan tube 5 blocks the blood from fouling either the protective foam 4 or the apertures 40. However, the actual size of the largest valves for use along the aorta is many times smaller than the valves have been shown for clarity in the drawing figures, so that unless an anticoagulant such as heparin, is released from an upstream jacket or through accessory channel 8, even a small amount of blood could prove a hindrance. However, an anticoagulant should be avoided in some patients, making an alternative approach necessary. Were blood to foul the protective foam 4 separating the valve from the substrate vessel, the valve is supplied with replacement foam.

If used, the anticoagulant is released just before ostium obturator 30 is advanced into the native lumen 1. The advancement of ostium obturator 30 then sweeps the anticoagulant forward to wet the cut edges of the ostium. Ostium obturator thus serves three functions:
1. In advance of actuating diversion chute 18 it squeegees an anticoagulant or any other drug in fluid form through the ostium, thus immediately medicating the tiny perforating wound created.
2. While held against the proximal intima of the substrate native lumen, ostium obturator 30 prevents blood from leaking into trepan tube 5, and
3. It defines by assisting to separate into separate flow columns, hence, the relative proportion, of blood to be diverted through trepan tube 5 and that to continue downstream past it.

When fully automated, the servomotor may be programmed to retract ostium obturator 30 immediately upon insertion of ostium obturator 30 through the ostium just enough to assure that ostium obturator 30 rests flush against the proximal internal surface of the substrate native ductus. Alternatively, the section pump can be used to generate a vacuum that pulls the obturator flush over the ostium. Normally, the process can proceed without interruption. If for any reason, however, the operator sees that a completely flush fit has not been accomplished, the direct manual fine control device, used with the power turned off, can be used to prevent even a negligible amount of blood from escaping. Precautions notwithstanding, should blood enter the foam in an amount that could significantly foul its permeability, the foam affected is quickly replaced with small pieces thereof supplied with the valve.

To allow the operator to switch between right and left-hand sides for direct observation of the direct manual fine control device mechanism from either side as essential to direct the side stem 19 to the right or left, for example, the device housing is transparent, and an engagement arm consisting of arms 77 and 77′ with end receivers 78 and 78′ makes it possible to alternately engage and disengage lead nut 72 so that rather than functioning as a screw, it rotates lead nut 72 connected to a locking mechanism in the form more or less of a capital letter Y, where either arm allows such engagement each on its respective side. Engagement arm piece 88 is made in one piece of die cut stainless steel or titanium alloy sheet stock, is burnished, and projects left and right-hand arms 77 and 77′ upwards.

### Internal Structure of the Direct Manual Fine Control Device

As shown in FIG. 10B, left-hand arm 77 and right-hand arm 77′ further diverge toward their respective sides as these rise, and at their upper ends, engagement arm piece 88 connector 76 catches 78, a triangular socket, on left arm 77 and 78′ on the right arm 77, are poised so that much as a small socket wrench wraps about a bolt head, rotation of the engagement arm piece 88 as shown to the right causes left-handed arm triangular socket, or catch, 78 to engage the left side projection of stationary engagement arm piece connector 76. To not be deflected when engaged, the engagement arm piece 88 must be inflexible, to which end it is preferably die cut from stainless steel or titanium alloy sheet stock.

To securely retain the projection of the engagement arm piece connector 76 within triangular sockets, or catches, 78 and 78′ the sides of these are wide enough to form reentrants too deep to allow engagement arm piece connector 76 to slip out when driven. Turned clockwise with moderate force to prevent accidental rotation, control knob 71 overcomes its central detent (unshown as behind knob 71) to move engagement arm piece 88 until an audible click indicates that the right-hand detent has engaged.

Turning now to FIG. 10C, to rotate engagement arm piece 88, lead nut 72 must be locked to control knob 71, while to rotate leadscrew 73 so that lead nut 72 can move along leadscrew 73, adjusting the proximal end of motor shaft 39 and therefore diversion chute 18 without at the same time rotating engagement arm piece 88 requires that lead nut 72 be unlocked from control knob 71. That is, leadscrew 73 either rotates together with lead nut 72 and engagement arm piece 88, or lead nut 72 is disengaged from the engagement arm piece, allowing engagement arm piece 88 to move along leadscrew 73 to extend diversion chute 18 into the substrate native lumen or to retract diversion chute 18.

FIG. 10C shows push/pull control cable knob 79 at the center of screw rotation control knob 71 when depressed overcomes the restorative force of small compression spring 81 to drive a Bowden cable-like flexible push-pull control wire forward so that its distal end is inserted thru leadscrew 73, then curves upwards into control wire receiver 75 locking knob 71, leadscrew 73, and lead nut 72 so as to rotate the engagement arm piece 88 as a single part.

To allow its removal once ostium obturator 30 has been correctly positioned flush against the proximal intima of the substrate vessel, thus covering over the ostium, the direct manual fine control is housed in a separate enclosure 91 attached to the bottom of enclosure 92 housing servomotor 37. Attachment of enclosure 91 to enclosure 92 is by insertion of pawl 93 into the bottom of a complementary receiving channel at the junction of left and right-hand engagement arm piece arms 77 and 77′. From FIG. 25, it will be clear that characterization of the enclosures as that upper or lower pertains when attachment to the substrate ductus is not inverted as are valves 89 and 90.

To allow the direct manual fine control device to be quickly and easily attached to and detached from the upper enclosure 92 housing servomotor 37 at a single point, engagement arm piece 88 must be positioned firmly enough to allow the insertion and withdrawal of intromitting pawl 93. To this end, the intromittent pawl receiving opening, or pawl receiver, at the bottom center of engagement arm piece 88 is tapered, or flared, wider at the bottom and curves upward to guide intromitting pawl 93 into a close enough relation to the opening that the operator need not direct undue attention or engage in an effort of precision to insert it. Shown in FIGS. 10C and 10E, the intromittent pawl receiving opening at the bottom center of engagement arm piece 88 then rises upwards into a friction fitting upper section of pawl receiver 99.

To meet these requirements, engagement arm piece 88 is suspended in its starting and ending disconnected and centered position shown in FIG. 10B by a miniaturized version of a miniature endoscopic-type grasper indicated in FIG. 10B with more detail provided in FIG. 10E and implemented as described below. The engagement arm piece must also be free to move from side to side to engage engagement arm piece connector 76 from either side as well as move with lead screw 72.

Accordingly, miniature endoscopic-type grasper 96 stably maintains engagement arm piece 88 in position, allowing the operator to insert intromittent pawl 93 into the bottom of engagement arm piece 88 and withdraw it once the adjustment in the horizontal position required has been completed, whereupon pulling down on enclosure 91 withdraws pawl 93 from the pawl receiver at the bottom center of engagement arm piece 88 for the procedure to proceed.

Grasper 96 is mounted to the side of manual control device housing 91, its shaft push/pull knob outside and shaft and grasper clamping pads, or jaws 142 inside housing 91 by a grommet-like surrounding ring, or torus, of stainless steel or titanium. The internal surface of the ring is broad enough to assure fitting about the grasper shaft as to disallow any play of grasper clamping pads 142 due to off-axis displacement at the ring when loaded and along with the shaft, is polished for nonresistant sliding to allow the clamping about and retreating of grasper clamping pads 142 from engagement arm piece 88.

Grasper shaft movement forward into manual fine control device housing 91 to stabilize engagement arm piece 88 by seizing it between grasper clamping pads 142 of grasper 9% before use so that insertion and removal of intromittent pawl 93 into the engagement arm receiver at its bottom center and will be quick and easy is limited to 5.0-7.0 millimeters by detents 94 outside enclosure, or housing 92 just in front of the grasper push/pull control knob 97 at the proximal end of grasper shaft 98, the other detent 95 along grasper shaft 98 inside housing 92. As indicated above, prior to use before engagement, arm piece connector 76 is shown in FIG. 10B ready for engagement either by engagement arm piece connector triangular socket, or catch, 78 on left arm 77 and catch 78′ on the right arm 77″before and after use in FIG. 10B.

Grasper 96 requires only one degree of freedom, that reciprocal to move its grasper clamping pads 142 to and away from engagement arm piece 88, positioned along the axis of movement at the displacement where the ostium obturator 30 will most often fit flush over the side-entry wound, or ostium, so that engagement arm piece 88 remains fixed in position for connection of the direct manual fine control device by soundly grasping engagement arm piece 88 between its grasper clamping pads 142. Requiring only reciprocal movement, grasper % is rigidly constrained to eliminate radial deflection.

Insertion and withdrawal of intromitting pawl 93 into the engagement arm receiver at its bottom center is manual. Thus, attaching and detaching the separably housed manual control device from engagement arm piece 88 is accomplished by the same action that connects engagement arm piece 88 for use. Grasper clamping pads 142 have miniature high coefficient of friction rubbery-lined rectangularly shaped grasper clamping pads 142 where the pad lining material can include vacuities to increase retention through the suction created when compressed and are dimensioned to interface with most of the engagement arm piece arm 77 at triangular socket, or catch, 78 or 77′ at catch 78′.

Engagement arm piece 88 is held rigidly in place while clamped between the grasp grasper clamping pads 142 by a spring powerful for its size, the type spring, located at the junction of the linkage at grasper clamping pads 142, noncritical. To facilitate its quick location and use later by the operator or an assistant, grasper shaft push pull control knob 97 extends out from the side of servomotor housing 92 to remain ready for later reuse when the shaft is retracted and the manual fine control device, no longer needed, is pulled off from the servomotor housing. No separate control to open and close grasper clamping pads 142 as might hinder the user are required at grasper knob 97.

Instead, a small rod inside the grasper shaft is connected to a linkage familiar to those in the art which opens grasper clamping pads 142 as the edge of engagement arm 77 is approached, and when contacted, a small spring-loaded bar at the junction of grasper clamping pads 142 is depressed to release grasper clamping pads 142 under the restorative force of the spring thus tripped. The unilateral grasper is used when attaching manual fine control deice housing 91 to motor housing 92 before use while outside the body and is easily retracted after use when inside the body by nudging up and slipping a finger the valve to retract grasper clamping pads 142. The linkage is so devised that rather than closing against the sides of engagement arm 77 in scissors fashion as would result in an uneven clamping force, grasper clamping pads 142 remain in parallel relation.

Withdrawal of the grasper by pulling out its push/pull control knob reverses this action, that is, opens grasper clamping pads 142, releasing engagement arm 88. FIG. 10C shows that in order to retract control wire 80 from lead nut 72, thus disconnecting lead nut 72 from engagement arm piece 88, freeing lead nut 72 to pass along leadscrew 73, push/pull control cable knob 79 must be pulled out as far as push/pull control cable knob 79 will allow, thereby retracting control wire 80 entirely from wire receiver 75 connecting engagement arm piece 88 to lead nut 72 as well as lead nut 72. This allows rotation control knob to rotate lead screw 73 rather than rotate lead screw 72 with engagement arm piece 88.

FIGS. 10A thru 10E show a vascular servovalve with direct manual fine control device attached and double outlet suitable for allowing a carotid endarterectomy free of complications due to ischemia, adverse fluctuations in blood pressure, as well as the risks of cerebral or myocardial infarction. In FIG. 10A, part number 82 is the male and 83 the female components of the bypass-line connector from the right common carotid to the internal carotid, and 84 and 85 respectively, those for connection of the bypass line to the external carotid. Part number 86 is the line to the internal carotid, and 87 that to the external carotid. The connections of the bypass lines from the common to the internal and external carotids is shown in FIG. 25.

To facilitate use of control knob 71 when the small button at its center must be depressed to lock lead nut 72 to engagement arm piece 88, rotation control knob 71 has projections 74 about its periphery. Accordingly, rotation of control knob 71 while depressing its central push/pull control cable knob 79 to the right as shown in FIG. 10B thus rotates engagement arm piece 88 to the right causing triangular socket, or catch, 78 to engage engagement arm piece connector 76 on the left, while rotation of engagement arm piece 88 to the left causes triangular socket, or catch, 78′ to engage the engagement arm piece connector 76 on the right side of engagement arm piece connector 76.

Rotation control knob 71 is fixed at its center position by a detent comprising a depression and complementary projection sufficiently retentive to preclude its inadvertent rotation. Accordingly, while push/pull control cable knob 79 is not depressed, rotation control knob 71 cannot rotate engagement arm piece 88 shown in FIG. 10B connected to leadscrew nut 72 in order to rotate engagement arm piece 88 with it but rather rotates leadscrew 73.

That is, when rotation control knob 71 is rotated clockwise while small button 79 is not depressed, rotation control knob 71 rotates leadscrew 73 to drive lead nut 72 and therewith, accessory channel-feeding drugline 8 forward, hence diversion chute 18 further into lumen 1 of the substrate ductus, typically an artery. Turning knob 71 counterclockwise while push/pull control cable knob 79 is not depressed does the opposite, that is, retracts diversion chute 18.

FIG. 11 provides a schematic representation of solenoid flow diversion jackets or vascular servovalves as fully retracted, or open, on the left to allow nondiverted flow, and on the right, fully extended, or closed, to divert all flow from the substrate to the other vessel. With solenoids movement is limited to full extension or retraction to remedy a transposition of the great arteries when the defect can be addressed without cardiac entry or to accomplish a sudden switch transplant. The part numbers consistent among the drawing figures, 8 are the accessory channels, 9 and 9′ the directionally distinguished mainlines, and 18 the diversion chutes. In FIGS. 11, 12, and 13, 41 and 41′ points to directionally distinguished stream or flow lines.

To best facilitate application, jackets with the aid of a straddle clamp as shown in FIG. 4 or incorporating an ostium obturator 30 and servovalves can be positioned along their respective ductus before or after connection of the jackets or valves to their respective mainlines 9 and 9′. The arrows at the center denote that the action is reversible. When used for reciprocal cross-circulation during a switch transplant, this reversibility allows the immediate truncation, or ‘bailout’, of the operation by fully extending the flow diversion valves if a highly damped nonsparking solenoid-driven sudden switch, or a variable retraction is a servo-driven metered switch, usually, with ensuing resumption.

Differing from plunger solenoid-driven diversion jackets in allowing instantaneously continuous adjustability between full extension and retraction, the applicability of vascular servovalves extends to metered compound vascular bypass, or sudden switched-organ transplantation whereby an implanted microcontroller dynamically adjusts the valves to the achieve optimal acceptance by the recipient based upon the data provided to it by implanted physiological sensors.

Mainlines 9 should be kept as short as possible by positioning the donor and recipient to best accommodate the chirality only when the difference in distance is significant. Accordingly, whether mainlines 9 and 9′ crisscross or decussate in bias depends upon how the subjects are disposed in relation to one another. The directions of accessory channels 8 serves pictorial clarity; in fact, these are routed as least disturbs neighboring tissue.

FIG. 12 shows the use of bidirectional side-entry diversion servovalves to take the place of separate valves as shown in FIG. 11 where, in particular with neonates and infants, space to accommodate separate valves to perform a metered switch transplant is at a premium. Whereas FIG. 11 shows the separate valves switched from fully retracted from the substrate lumen, or closed on the left, thus playing no part in the normal flow of blood through the substrate lumen, and fully extended, or open for full flow diversion on the right, FIG. 12 shows the bidirectional valves at the half-way setting to allow half the flow to be exchanged between recipient and donor and half to continue the flow of blood native to the subject, the extent of mixture throughout either circulatory system contingent upon the duration of exchange.

When the defect does not reach down into the roots of the aorta and pulmonary artery, the same arrangement using solenoid-driven diversion jackets is suitable for the extracardiac correction of transposition of the great arteries in a neonate or infant. The half-way position denotes servovalve rather than solenoid control, this position exemplary for what is actually the continuous variability essential for metered compound vascular bypass, or sudden switched-organ transplantation. The use of bidirectional rather than separate servo-driven valves indicates that depicted is a moment in a metered transplant in a neonate or infant.

The valves controlled in a complementary manner, a complete shutoff or lack of outflow equates to the concurrent lack of inflow; otherwise, the volume of blood outflow equals the volume of inflow. Were the valves used in a neonate or infant to perform an extracardiac transposition of the great arteries, an adverse immune reaction would not be involved, so that to adjust and monitor the response would not be necessary. Bidirectional diversion chutes can also be incorporated into highly damped nonsparking solenoid-driven diversion jackets where the space-saving attribute facilitates the placement of duplicate jackets.

For example, shown in FIG. 13B, used to exchange flow between the great arteries when permanent placement to correct a transposition is needed, any irritation is avoided by periodically switching the exchange of flow between upper and lower pairs of jackets. If retained in fully extended or retracted position by detents, the push-pull highly damped nonsparking plunger solenoids require a momentary surge of current to overcome. Irritation and displacement are countered because:
1. The jackets are lined with a relatively thick layer of viscoelastic polyurethane foam.
2. Perforations through the jacket shells 3 and foam 4 expose sufficient surface area of the substrate ductus adventitia to the internal environment to avert the otherwise inevitable and quick atherosclerotic degeneration associated with complete enclosure.
3. In larger jackets that afford adequate clearance to allow the delivery to the adventitia of medication, druglines 8 inserted into accessory channels can directly release a steroid, for example.
4. The jacket spring hinges are specified to comply with minimal resistance to the pulse of the substrate artery while applying sufficient grasping force to avert displacement, or migration, along it.
5. The suture loops 15 at the abaxial or butt end of the valve side stem and at other points about the outer shell 3 allow connection to nearby minimally or noninnervated connective tissue to stabilize the jacket from levering with the pulse.

For these reasons, irritation, if it occurs at all, should arise only after long intervals, at which time the remedial measures are noninvasive. Lacking the versatility of servovalves, plunger solenoid-driven diversion jackets still allow an exchange of blood within one or between two bodies to be stopped immediately. To accommodate the quick pace of growth at the outset of life, jackets are lined with somewhat thicker highly compliant viscoelastic polyurethane foam 4 and sprung hinges 11, and mainlines 9 made of highly elastic and usually accordioned tubing. At the small diameters of the mainlines as bloodlines, the use of valves with accessory channels to directly target heparin, for example, is necessary.

FIG. 13A shows an arrangement of bidirectional diversion jackets for the extracardiac correction of a transposition of the great arteries, or use as diversion servovalves to perform a metered organ transplant, such as of the heart where space is limited in a prenate, neonate, or infant, for example. FIG. 13B shows the doubling of this arrangement at an interval along the arteries so that flow can be switched between the upper (superior, cephalad, craniad) and lower (inferior, caudad) pairs of jackets in a transposition repair or valves in a metered switch transplantation as one of several measures enumerated just above in this section to counter irritation of the substrate artery. Part numbers among the various drawings are consistent.

FIG. 13A shows paired bidirectional diversion jackets without duplication along the same ductus at a nearby level, and FIG. 13B shows paired bidirectional diversion jackets with duplication. The purpose in duplication is to allow flow to periodically be switched between the upper and lower pairs. Pairing is considered only when there is concern that despite the compliant lining of the jackets and the release of an emollient onto the substrate adventitia, permanent placement along the forcefully pulsatile great arteries and the antimigration and stabilization use of suture loops 15 could result in irritation due to the forcefulness of the compressions associated with the systoles. Such arises when bidirectional diversion jackets are used to correct a transposition of the great arteries where an extracardiac repair is practicable but the combination of any constantly repetitive levering movement and expansion of the vessel within the valve could cause irritation.

FIG. 14 shows a vascular servochoke. The miniature tubular pulse motor shown in FIG. 9 comprises two major outwardly evident components, shaft 42 and forcer 37. To prevent irritation to neighboring tissue, the corners and edges of the device housing (enclosure, case) are rounded. The adventitia is contacted entirely around only by felt lining 4, and rather than fully enclosed as would cause intimal degeneration, exposed to the surrounding cavity through numerous perforations 40 placed all about the jacket. As in all side-entry devices, outer shell 3 is never allowed into contact with the adventitia. Various means for providing a drugline to deliver mediation directly to the adventitia are addressed above under Background of the Invention.

In FIG. 14A, part number 43 is the chokeplate (choke plate, obstruction plate, flat obstruction plate, obstructor, obturator) and part number 10 is a lock nut cap that gives access to the motor. Accessory channel 8 is shown entering with outlet into open cell viscoelastic polyurethane protective foam 4, releasing medication for diffusion to the adventitia. Accessory channel 8′ is shown entering obstruction plate 43 from below, releasing medication to flow through and out of obstruction plate 43 bottom pore 44. Dishing out, that is, creating a depression or concavity on the underside of obstruction plate 43 increases the impedance to flow, which can be used, for example, to increase uptake of the drug released through bottom pore 44, the impedance variable according to the depth and form of the depressions.

Flowing against gravity through a capillary tube-gauged catheter serving as accessory channel 8′ requires not only pressure equalization but increased rate and pressure of delivery by the outlet pump or drip stopcock of the small drug reservoir implanted in the pectoral region. One function of a servochoke to increase the pressure of the oncoming flow, assuming flow is upward (cephalad, craniad) through native lumen 1, chokeplate bottom pore 44 can be used to release a nanoparticle carrier-bound drug which the choking action will pressurize for facilitated uptake in the intima. Where uptake is still inadequate, a magnetized perivascular jacket, described and illustrated in U.S. Pat. No. 11,389,171, entitled Integrated System for the Infixion and Retrieval of Implants, is positioned along the segment to be penetrated, the nanoparticulate then superparamagnetic.

Radiation shielded delivery lines are described and illustrated in U.S. Pat. No. 11,013,858, entitled Nonjacketing Side-entry Connectors and Prosthetic Disorder Response Systems, and in U.S. Pat. No. 11,759,186, entitled Ductus Side-entry Jackets and Prosthetic Disorder Response Systems. Allowing the irradiation of ductus-lining epithelia, endothelia, or urothelia is exceptional and essentially limited to short segments of vessels intimately associated with malignant tissue, and then in the elderly. Such application is rarely other than endoscopic or long-term. Obstruction plate 43 is introduced into the substrate native lumen 1 through a stab wound, eliminating the need for a trepan tube and thus allowing a significant reduction in size.

The action can be accomplished so quickly as to minimize if not eliminate any seepage of blood or the need for a straddle-clamp, one configuration thereof shown in FIG. 4, to define the segment for entry. Radially symmetrical, hence, neutral as to antegrade or retrograde flow, a servo chokevalve can be positioned along any vessel without regard to the direction of flow through the substrate vessel. Not requiring a well cut round entry aperture, the vascular servochoke shown in FIG. 14, chokeplate 43 is inserted through substrate lumen wall 2 through a stab wound which the operator should be able to place so quickly and with as little loss of blood as to not even require the aid of a clamp.

On the unseen backside of the servo-driven chokevalve shown in FIG. 14, a drugline enters servochoke side-stem 19 through the outer jacket 3 and foam 4 to inlet chokeplate 43. Passage through chokeplate 43 as an accessory channel is through a micromachine milled channel created when chokeplate 43 is bonded together from an upper and a lower layer as two complementary plies. The accessory channel outlet is beneath chokeplate 43 at 44.

The fluid drug and electrical lines having entered on the unseen or far side of the chokevalve, accessory channel outlet 44 drips drugs which the disruption in streamline flow backscatters for uptake into the endothelium 13 and 14. Other part numbers are consistent with those in the preceding drawings. Vascular servochokes (choke servovalves, servo chokevalves, choke-valves) are used primarily to raise the local blood pressure to increase the upstream uptake of drugs released into the vessel, usually a vein with lower blood pressure, a moment earlier.

A perivascular magnet jacket shown in FIGS. 2 thru 7 and described in U.S. Pat. No. 11,389,171, entitled Integrated System for the Infixion and Retrieval of Implants, allows increased uptake of superparamagnetic nanoparticle-carried drugs, the more so when combined with an upstream servo chokevalve to reduce the post valve blood pressure, allowing extraction with a smaller, less powerful but less obtrusive magnet. The drug may be one systemically circulated where higher uptake is wanted along the upstream segment. Release of the drug is through an accessory channel with outlet beneath the chokeplate or from an accessory channel in an upstream jacket or valve, larger volumes of an agent delivered through the its mainline. Another type of choke servovalve is the sphincteric, shown in FIGS. 11 and 12 and described in U.S. Pat. No. 11,759,186, entitled Ductus Side-entry Jackets and Prosthetic Disorder Response Systems.

Servo chokevalves are always driven by a servomotor automatically controlled by an implant microcontroller, or in comorbid disease, a microprocessor, serving as the master controller in a hierarchical control system in accordance with physiological data fed up through the hierarchy to the master controller which executes a prescription-program devised for the specific patient. The microcontroller or microprocessor controls a small peristaltic pump at the outlet pump or drip stopcock of a small flat drug reservoir implanted subcutaneously in the pectoral region.

### Valve Applications as Shown in FIGS. 15A Thru 17 and 22 Thru 25

FIG. 15A is a simplified schematic representation of a switch transplantation, in which blood flow through the actual vessels of the native solid organ-here the heart—have been schematically summarized to make clear how flow is switched from the native to the donor organ. Depicted as applied to the heart, the same process pertains to a kidney, liver, lung, gland, or any other graft organ. Heart outflow is through the aorta and pulmonary artery (pulmonary trunk, main pulmonary artery) and inflow through the venae cavae. In an orthotopic switch transplantation, once all blood flow is through the donor organ, the native organ is removed. Alternatively, blood flow can be continued through both native and donor organs indefinitely, thus adding the donor organ as supernumerary in a heterotopic transplant.

Shown is the compound vascular bypass transfer of blood flow from the native to the donor organ at the half-way point, 45 representing the transections at the ends of the stumps of the donor graft organ. The chutes have been depicted at an intervening point in extension instantly traversed in a sudden switch transplant but the midpoint intervening between complete retraction with the organs independently perfused and complete extension during which both organs are supplied blood by both the donor on life support and the recipient to blend the blood in order to effectuate a measure of immune tolerance induction. More specifically, in either a metered switch, or compound vascular bypass orthotopic or heterotopic double heart transplant, the flow of blood is directed as shown in FIG. 15A.

FIG. 15B shows the bloodline and valve connections needed to simultaneously retrieve a second heart which had been implanted in a patient with advanced heart failure where the native heart had recovered, for example, with no interruption in blood flow for transfer into the circulatory system of a second recipient. FIG. 15C shows the valve and expandable confluence conduit connections needed to group the tiny vessels in a neonate, for example, without the procedural duration that would result if each vessel had to be separately connected, and FIG. 15D shows the components of the tiny vessel grouping or gathering device.

In an orthotopic transplant, the point at which the servovalve diversion chutes are midway along their overall throw is also the halfway point in transferring the graft organ from the circulatory system of the donor into that of the recipient. In a heterotopic double heart transplant, this is the end-point, at which the donor heart is removed and implanted in the recipient. The essential purpose in the operation being to increase the pool of usable hearts, the exact point is that at which a less than ideal donor heart most closely matches the heart of the recipient in ejection fraction, or absolute stroke volume. This determination is entrusted to the implanted master control microprocessor on the basis of feedback from implanted pressure sensors.

This determination precedes a second determination as to the point of optimized homeostasis in terms of immune reconciliation. That is, once the end-point based upon the midprocedural matching of ejection fractions has been determined, the end-point for the optimized release of anti-inflammatory and immunosuppressive medication, likewise dependent upon sensor feedback, continues until the overall best combination of heart function and minimized reactivity has been established. Postoperatively, the process of stabilization at the point of optimized homeostasis through the release of medicinals and counteractants thereto continues.

The disorder response system fully implanted, this process of monitoring and effecting adjustment as necessary will remain in effect for both power matching should either or both hearts change in ejection fraction or should a rejection reaction appear. It is possible that with the aid of an organic ‘ventricular assist device’ and immediate medicinal support the native heart will have a partial recovery. If and only if the degree of improvement is substantial, consideration should be given to the sustainment in improved performance were the donor heart to be explanted. Due to the cost and pain involved and the fact that the implanted system will spontaneously adjust its treatment to accommodate the instant state of overall cardiac performance, this would be done rarely and then only if the donor heart could save another patient.

### Tiny Vessel Grouping Device

Many vascular valves will not require all of these features and will cost less. In dimensions and materials, all components for internal placement are made preferably transparent and as small and light in weight as possible, often from preferably clear transparent polyetheretherketone or polypropylene, for example. While producible in microminiature sizes, the valves needed to perform a compound vascular bypass heart transplant in a late prenate, neonate, infant, or early toddler would number nine or more, would be costly to produce, and to connect each with the aid of a binocular microscope separately would considerably increase the duration of the operation to impose additional stress on the already frail patient.

The grouping of tiny oxygenated blood carrying vessels to expedite the completion of a compound vascular bypass heart transplant is addressed above in section 2b(1)(b)(b14), entitled Confluence Conduits: Connection of One or More Bloodlines to Flow through a Common Channel to the Same or a Different Number of Bloodlines with Self-leveling Apportionment of Outflow, in the closing subsection thereto entitled Tiny Vessel Grouped Inlet/Outlet Expandable Confluence Conduits. As shown in FIG. 16, the vena cavae (and the azygos veins not included) few, the need for grouping tiny vessels pertains only to those conveying oxygenated blood. In FIG. 15C, these flow through the upper expandable confluence conduit. This means that the lower conduit does not involve grouping or the application of grouping devices.

To avoid this eventuality, grouped vessel inlet/outlet confluence conduits devised to expand with growth as delineated above in section 2b(2)(f), entitled Expandable Structure to Accommodate Growth and Self-adjust in Size in Vascular Segment Prostheses, Lines, and Confluence Conduits Placed in Children, are provided to allow tiny vessels alike in conveying oxygenated or deoxygenated blood to be gathered together for flow past a common diversion chute controlled by a single larger valve.

This is shown in FIG. 15C and addressed above in section 2b(1)(b)(b14), entitled Confluence Conduits: Connection of One or More Bloodlines to Flow through a Common Channel to the Same or a Different Number of Bloodlines with Self-leveling Apportionment of Outflow, in the subsection thereof entitled Tiny Vessel Grouped Inlet/Outlet Expandable Confluence Conduits, as well as below in this section. FIG. 15D shows the components within the tiny vessel grouping or gathering device.

FIG. 15C tracks FIG. 15A in showing the positioning of tiny vessel grouping devices in the context of grouped or gathered inlet/outlet expandable confluence conduits described above in section 2b(2)(f), entitled Expandable Structure to Accommodate Growth and Self-adjust in Size in Vascular Segment Prostheses, Lines, and Confluence Conduits Placed in Children, as well as in this section and shown in FIGS. 15C and 36A. Grouping devices are for use in performing a heart transplant in the smallest pediatric patients so that blood flow through the tiny vessels, as exemplary, shown as three, past or through a common flow diversion chute eliminates the need to separately connect each of the vessels, which significantly extending the procedural duration, causes excessive stress on the already frail patient.

The blood of vessels flowing into a confluence conduit becoming mixed, while complementary in the sense of mediating the blood supply and drainage as shown in FIG. 15C, arterial and venous vessels are grouped separately for flow into exclusively oxygenated arterial or deoxygenated venous confluence conduits. An exception in the pulmonary artery, which transmitting deoxygenated blood, if grouped at all, must be grouped with veins.

The schematic representation makes it look as if the three vessels are the supraaortic branch arteries, which could but need not be the case. In fact, in this summarizing representation, any three outlet vessels can be inferred. An immediately related feature are the flats and vaults expandable configured confluence conduits 230 and 230′ which designed to expand in step with the growth of the patient significantly postpone if not eliminate the need for later reentry. Omitted in FIG. 15A, the orientation of the valve sidestems part number 19, is shown in FIG. 15C. FIG. 15D provides a detailed view of the tiny vessel grouping or gathering device indicated only by labeled boxes in FIG. 15C.

The reasoning behind the device of grouped or common diversion chute-equipped compound vascular valves is specified above in sections:
2a, entitled Background of the Invention. General;
2a(1), entitled Congenital Defects of the Heart and Other Organs: The Importance of Prompt Intervention; 2b(1), entitled The Intercorporeal Diversion of Blood Flow;
2b(1)(b)(b2), entitled Prenatal Compound Vascular Bypass, or Switched-organ, Transplantation of the Heart;
2b(1)(b)(b3), entitled Neonatal and Infant Compound Vascular Bypass, or Switched-organ, Transplantation of the Heart;
2b(1)(b)(b5), entitled Heterotopic Double Heart Metered Compound Vascular Bypass, or Switched-organ Transplantation [and so on];
2b(1)(b)(b14), entitled Confluence Conduits: Connection of One or More Bloodlines to Flow through a Common Channel to the Same or a Different Number of Bloodlines with Self-leveling Apportionment of Outflow;
2b(1)(b)(b16), entitled Direct Aortocaval Connection: Unified Entry Arterial and Exit Venous Confluence Conduits for the Heterotopic Connection of a Graft Heart in the Lower Abdomen;
2b(2)(b), entitled Bidirectional Valves for Perinatal Extracardiac Correction of Complete or Dextro-transposition of the Great Arteries and Reciprocal Cross-circulation;
2b(2)(f), Expandable Structure to Accommodate Growth and Self-adjust in Size in Vascular Segment Prostheses, Lines, and Confluence Conduits Placed in Children;
2b(2)(h), entitled Local Vascular Pressure Relief; Section 4, entitled Objects of the Invention; as well as in
This section, 6, entitled Description of the Preferred Embodiments of the Invention.

FIGS. 15C and 15D show the positions of the grouping devices as that of the arterial vascular valves. Since the distance between these valves on the vessels of the donor and recipient is through catheteric tubing confluence conduit 230 connected between the outlet sidestem of the sending valve and the inlet side of the receiving valve, the tiny dimensions of the vessels themselves is not a factor as would detract from this relatively distant spacing. If this distance necessitates the addition of propulsive force in addition to the intrinsic force of the pulse, the positioning of an inline axial pump such as those shown inside arterial confluence conduit 230 as part number 167 in FIG. 35B, for example, satisfies this need.

For clarity, FIGS. 15C and 15D are shown without the atheroma-counteractive jacketing applicable to all side-entry devices, which as seen in FIGS. 5, 7, 8, 10A, and 10F thru 10H, includes through and through apertures 40 and dissolution countering coated viscoelastic polyurethane foam lining 4 to prevent compression of the fine nervelets and arteries about the adventitia substrate vessels.

In a metered switch transplant, this intervening point in chute extension may be temporary, the chutes partially retracted or further extended from it, or can represent the final setpoint, adjusted for a mismatch in the relative size, consistent with the absolute stroke volume, or ejection fraction of either heart. This midway point interposed between donor-recipient circulatory independence and blending is shown for arterial flow in FIG. 20B and for venous flow in FIG. 21B. The vessels involved are identified in FIG. 16. The extent to which the blood is blended is controlled by the duration the diversion chutes are positioned at such an intervening extent.

The progression of the arterial diversion chutes depicted in FIGS. 20A thru 20C and the venous diversion chutes depicted in FIGS. 21A thru 21C is not ‘dumb’ in relentlessly extending further into the lumina of the substrate vessel despite the detection of a more pronounced immune response but rather is directly responsive to such an indication received by the fully implanted prescription-program executing disorder response system master control microprocessor from rejection sensitive sensors such as specified above in section 2a, entitled Background of the Invention. General.

Sensor feedback indicative of an adverse immune response will cause the chutes to pause advancing, the remedial medication prescribed is released through the servovalve accessory channels, and the process slowed down or suspended until spontaneously of medicinally resolved. The controller can also halt the process and signal the operator of a need for a medical resolution based upon clinical judgment. In an orthotopic transplant, excision of the donor organ is not performed until blood flow has been passed to it exclusively.

That except for being stopped at the halfway point with the recipient heart having been retained, the process for a double heart metered transplant is no different than that for a single heart transplant. Accordingly, a figure that represents both donor and recipient hearts remaining in place with the arterial and venous vascular servovalve diversion chutes at the half-way point to equally apportion flow through both hearts indicates that this is a metered switch heterotopic, or double heart transplant. The end product of such a transplant when the added heart is positioned in the chest is depicted in FIGS. 16 and 17A.

Alternative positions for the second heart are addressed above in sections 2b(1)(b)(b5), entitled Heterotopic Double Heart Metered Compound Vascular Bypass, or Switched-organ Transplantation and the Use of Unified Entry Arterial and Venous Exit Confluence Conduits to Avoid Disuse Atrophy when the Graft Organ is Positioned in the Lower Abdomen by Direct Aortocaval Connection; 2b(1)(b)(b16), entitled Direct Aortocaval Connection: Unified Entry Arterial and Exit Venous Confluence Conduits for the Heterotopic Connection of a Graft Heart in the Lower Abdomen; 2b(1)(b)(b17)., entitled Doubly Heterotopic Double Heart Compound Vascular Bypass, or Switched-organ Transplantation; and 2b(1)(b)(b18), entitled Hybrid Doubly Heterotopic Double Heart Transplantation.

Increasing the pool of replacement hearts, the added heart is usually similar in size to that of the recipient; however, size matching is not necessary. The continuity of circulation while the donor is kept past death on life support in the same center eliminates storage, and therewith, the endothelial degeneration and cellular breakdown products which contribute to early hyperacute rejection and late-term vasculopathy. That neither native nor donor organ are incised, the recipient placed under regional, not general, anesthesia without cardiopulmonary machine support, and can remain conscious throughout the procedure likewise offer a cognitive benefit over conventional transplantation.

In a metered switch transplant, where the transfer of circulation from the native to the donor organ is gradual, a choice can be made at this intermediate stage as to whether the donor organ should orthotopically replace or heterotopically supplement the native organ. Part numbers are consistent with those identified previously. If supplementation is chosen, circulation remains divided between the native and the graft organ. An hypoxic condition of either organ is detected and signaled by implanted sensors to the implanted microcontroller which then toggles the diversion chutes to alternately apportion a nearly full complement of blood to either.

In contrast to metered compound vascular bypass, or sudden switched-organ transplantation, which also allows immune tolerance induction concomitant with organ transfer, a sudden switch transplant transfers circulation from the native to the donor organ abruptly and entirely without the ability to stop at an intervening point or reverse the diversion chutes, so that immune tolerance induction therapy must be pre- and postprocedural.

FIG. 16 schematically shows the flow direction lines and identifies each of the nine actual vessels summarized in FIG. 15A. In FIG. 16, the letter A stands for the aortic arch, LPA stands for the left pulmonary arteries; LPVs for the left pulmonary veins; IVC for the inferior venae cavae; SVC for the superior venae cavae; and RPVs for the right pulmonary veins. Arrowheads directed to the right denote outflow, those to the left inflow. The double heart transplant having been completed as depicted, flow through the vascular diversion servovalves have been brought to the halfway retracted or withdrawn from the lumina to pass half the blood of the donor through the recipient and donor hearts.

Schematic, FIG. 16 does not show the servovalve diversion chutes at the corners where flow between the donor and recipient hearts merge. This might leave the impression that flow arriving over each vessel at the corners or junctions can interfere with or oppose one another; however, FIG. 15A makes it clear that the servovalve diversion chutes merge the approaching flow lines with no directional opposition. When the hearts are free-running rather than electrically synchronized to beat together, the absolute stroke volume, or ejection fraction when the two beat simultaneously is increased. For the two to beat in intercalated rhythm may create the impression of a tachycardia with something lower than a normal ejection fraction. However, over an interval that allows several beats to pass, net performance is acceptable.

At eighteen, the number of valves required gives emphasis to the importance of achieving the lowest weight and greatest miniaturization of each, obtained primarily through microfabrication and the use of light plastic and metal parts and electrical windings of silver. In the smallest pediatric patients, the need to valve each vessel is eliminated through the use of tiny vessel grouped or gathered inlet/outlet confluence conduits shown in FIGS. 15C and 15D and explained above in section 2b(1)(b)(b14), entitled Confluence Conduits: Connection of One or More Bloodlines to Flow through a Common Channel to the Same or a Different Number of Bloodlines with Self-leveling Apportionment of Outflow, in the subsection thereof entitled Tiny Vessel Grouped Inlet/Outlet Expandable Confluence Conduits.

For visual clarity, the schematic representation of FIG. 16 also omits flow lines through the accessory channels, or druglines, associated with each valve. All lines fluid and electrical must be routed to avoid strangulating tissue. To implement this, accessory channels can enter into the valves at a number of places such as along circumferentially running perforations 40 or transversely to side stem 19 and into trepan tube 5 or foam 4. If direct routing will not allow this, then fluid and electrical lines are rerouted. When clearly safer for the patient, hard wires are replaced by remote radio channels such as ‘Bluetooth’ and transcutaneous energy transfer.

A metered switch solid organ transplant best is best accommodated through the use of vascular servovalves that allow direct manual fine control such as that shown in FIGS. 10A thru 10C so that the operator can make minor adjustments in diversion chute position during the condition of no current flow before the automated process of organ transfer is initiated. Were the hearts different in size, this valve setting would have been adjusted to either side of the halfway point to accommodate the size difference in absolute stoke volume, or ejection fraction, of either heart. In FIG. 16, the ‘corners’ at the connections of the mainlines, here bloodlines 9 to each of the vessels is the side-entry vascular servovalves by which the connection is made.

Since these valves not only mediate circulation postoperatively, but the transfer of circulation between the two in transferring the donor heart into the circulatory system of the recipient, only fully operational servovalves are used, disallowing the use of passive, or nonadjustable diversion jackets. This ability to apportion blood flow through either heart in accordance with the relative size of stroke volume of each heart eliminates the complications normally associated with hearts mismatched in size in heterotopic transplantation of a smaller donor heart, the use thereof making it possible to retain more of the right lung as preferred.

In FIG. 16, the number of bloodlines can be reduced through the use of one or more confluence conduits as described below in this section and shown in FIGS. 35A thru 35F, the use thereof making it easier for the operator and assistants to keep track of each line, allow the vessels connected thus to be specially treated, and facilitate connection between the recipient and donor organs when that of the recipient or both are anomalous in number. The thoroughness of perfusion obtained with the scheme shown in FIG. 16 and FIGS. 35A thru 35F with the use of one or more confluence conduits precludes the development of disuse atrophy as occurs when the graft heart is connected to the abdominal aorta and inferior vena cava as is commonly done in immunological laboratory testing, for example.

More specifically, in a double heart transplant, retention of the valves allows maximum flexibility in automatic flow adjustments, continued maintenance and drug delivery on an indefinite basis. For these reasons, the midprocedural number of valves, rather than replaced by basic side-entry jackets, for example, are best retained after the operation has been completed. However, this separation may actually ameliorate irritation associated with direct contact, and the immediate support of directly targeted drug delivery should overcome any drawback this separation might otherwise impose.

The need for 18 valves demonstrates the importance of achieving small size, light weight, and minimal cost in valves for use in such a double heart transplant, of which the advantages, enumerated herein, are numerous, substantial, and basic. With the diversion valves set at the halfway point, when both hearts beat simultaneously, the hearts divide the normal volume of blood equally. The servovalves can divide the full volume of venous return between the two hearts in any proportion, or the entire volume to either heart at a given moment, but cannot double the volume available to pass a full volume of blood to both simultaneously. To allow an almost full volume of blood to be processed by each in turn, the valves are switched to favor the one, then the other in alternation.

Cardiac hypovolemia is detected by oximeter implants in either heart and prevented by automatically initiated toggling of the servovalves to alternately favor either heart at the optimal rate the microprocessor is programmed to hunt for and set. Should a dysrhythmia arise, it is detected by implant sensors using circuitry much the same as that used in an implantable cardioverter-defibrillator and the disruption electrically resynchronized, or reversed through the application of shocks initiated at a level calculated to avoid injury, which is stepped up until successful.

Thus, to prevent the inducement of a dysrhythmia that could lead to a sudden arrest, and because for the heart to beat when empty can be injurious, sufficient blood is always provided to both to avoid these eventualities. Because this arrangement allows venous return and ejection to be apportioned between the hearts, the added heart can be and is best somewhat smaller in size to allow placement with the least removal of lung tissue. Lung tissue regenerates not by increasing in mass but rather by increasing the number of alveoli per cubic centimeter, making it doubtful that the patient will experience even a temporary postoperative shortness of breath worthy of note.

The graft organ should not interfere with the restoration of the ribcage to very nearly if not exactly its normal conformation before closing. Drug delivery responsive to implanted sensor input, all of the servovalves incorporate drug-delivery, or accessory channels, through which the control system can target drugs through catheters connected to each valve from small flat reservoirs placed subdermally in the pectoral region.

Drug replenishment is by injection into the reservoirs through a body surface port, of which FIG. 26A shows one injection type and FIG. 26C shows a combined injection and urine discharge type also shown in FIG. 28. FIG. 30 shows the addition of control knobs 153 and 153′ that make it possible for the patient to adjust valves 143 and 143′ of the kind shown in FIG. 5 by means of Bowden cables 28 and 28′. Other types of body surface ports are described and illustrated in FIGS. 27, 28, and 45 of U.S. Pat. No. 11,759,186, entitled Ductus Side-entry Jackets and Prosthetic Disorder Response Systems.

### Implantation of Body Surface Ports

Subcutaneously positioned body surface ports are shown in FIGS. 26A and 26C, with the reverse surface of a port such as that shown in FIG. 26A shown in FIG. 26B. Unlike commercial portacaths and mediports, these ports can give access into more than two openings into druglines, each identified by means of a tiny indicating tattoo dots on the skin. All ports are injected through a self-sealing membrane. When drugs must be injected frequently so that the skin would be injured, these openings are positioned epicutaneously and covered over by a protective cap lined with an open cell foam wetted with an antiseptic such as ordinary hand sanitizer when access is not needed.

Otherwise, the complete enclosure of the port openings beneath the skin offers a measure of protection against infection. For most placements, a part of the pectoral region where the skin is relatively lax is chosen. When necessary and time permits, expansion of the skin is carried out in advance. Most body surface ports about an inch in diameter, placement is through an incision of about one and one half inches. A sterile caulking or culinary (cutlery), or a large brain spatula is used to separate the skin from the underlying fascia in the direction-usually downward, toward the abdomen or pelvis—where the drugline or druglines, whenever and to the extent possible, will be subcutaneously tunneled from the port to the site for attachment of the terminal device.

Continued passage more deeply up to the site for connection of the terminal device-typically a side-entry flow diversion jacket, nonjacketing side-entry connector, or vascular valve, is accomplished laparoscopically. Druglines are made of resilient kink-resistant material and connected to the port by nonallergenic rubbery molded elbow-configured tube connectors. Referring to FIG. 26B showing the reverse, or back, side of a body surface port, the druglines are shown as part number 8. In FIGS. 26A and 26C, the body surface port backplates 100 are detachable and provided with eyelets or loops 104 to allow the use of suture to immobilize the port when reattached to backplate 100.

For attachment to the underlying tissue, body surface port backplate 100 is removed from the port, 146 in FIG. 26A and 147 in FIG. 26C. With backplate held a few inches apart from the tissue to which it is to be stitched, a half-circle surgical suture needle, typically, a number 7 or 8, is used to pass suture through eyelet 104, then into the subjacent tissue for return through the eyelet. A few loops of suture are passed through the eyelet and the tissue, the suture then left untautened. The same is done through the other eyelet. Once both eyelets have several turns of suture coursing through them and the underlying tissue, the suture is pulled snug and knotted to place the backplate stably against the subjacent tissue, the anterior part of the port is reattached to the backplate, and the incision closed, preferably with a surgical grade cyanoacrylate cement.

Placement of the suture eyelets 15 at the corners of and parallel rather than perpendicular to backplate 100 makes this process somewhat easier but necessitates eyelets at each of the four corners rather than two rather centered along the backside. Using a handheld sonograph, or echograph, probe (transducer), the exact positions of port openings 107 are marked on the overlying skin with tattooed dots. Additional information pertaining to each hole is best included in abbreviated form rather than consigned to written descriptive information more susceptible to human error.

FIG. 17 shows the positions of the hearts following a double heart switch transplant, the added heart whenever possible which is the usual case, having been placed in the chest as preferred for several reasons, to include protection by the rib cage as close to the natural position intended therefor, reducing the need to work against gravity, and for other physiological reasons. Positioning thus is overridden only due to deformity or a crushing injury to the right side of the chest where an urgent need for a heart outweighs the additional time it would take to reconstruct the damage. The vessels having already been identified in FIG. 16, to avoid clutter, only three recipient-donor connections have been shown.

Shown in FIGS. 17 and 27C, subcutaneously implanted multiple injection point port 122 of the portacath or mediport type corresponding to port 146 in FIG. 26A with on-the-skin, or epicutaneous injection point indicating marks 124 allows the replenishment of implanted drug reservoirs such as shown in FIG. 26B by multiple injection means such as those shown in FIGS. 27A and 27B through tattooed injection point indicators 124. Although to be certain that the multiple injector device is exactly positioned to replenish each drug through its intended injection opening only one injection point need be identified on the skin, here out of an abundance of caution, two injection points 124 are shown as preferred. The multiple injectors shown in FIGS. 27A and 27B comprise four injectors of which the injection openings 107 are shown in FIG. 26B, for which two of the injection points 124 are marked on the skin as shown in FIG. 27C.

While a port with drain 110 to the exterior such as shown in FIG. 26C for the outflow of urine would ordinarily be positioned to a side of the mons pubis where no pump is needed and the wearer has a clear view of it, where the frequent insertion of cabled diagnostic and/or therapeutic devices at a higher level is required, the same kind of port, shown as 147 in FIG. 26C, would normally be positioned in the pectoral region. Thus, depending upon the application, both ports with an opening to the exterior such as port type 147 shown in FIG. 26C and those fully subcutaneous such as port type 146 shown in FIGS. 17, 26A 26B, and 27 can be positioned at different levels.

FIGS. 18A and 18B depict the flow through one of the arteries shown in FIG. 15A in a sudden switch heart transplant. In FIG. 18A, highly damped nonsparking solenoid-driven flow diversion chutes are fully retracted, ostium obturator 30 covering over the side-entry opening along near luminal wall 13, thus blocking the outflow of blood through the ostium. When the operator of an assistant depresses a switch to send current to the solenoids at the donor and recipient ends of the mainline, or bloodline, connecting each of the vessels shown in FIG. 15A, all of the blood flow diversion chutes simultaneously snap from full retraction against near luminal wall 13 to full extension, whereby all flow is diverted from the recipient to the donor graft organ, here the heart, the ostium obturator now pressed flush out of the flow path against far luminal wall 14.

In FIG. 18A, arterial flow from the donor heart before the diversion chutes are deployed is summarized, in that all arteries are depicted as one on the left, and all flow from the diseased heart of the recipient is summarized on the right. Arterial flow after the chutes have been deployed is shown in FIG. 18B, where the X strike-out indicates that with the donor organ now incorporated into the circulatory system of the recipient, the innate organ has been bypassed and removed. FIGS. 5, 7, and 8 show the structure of intravascular diversion valves and servovalves. The sizes of the jackets vary according to those of the respective substrate vessels, the rotational orientation chosen to least encroach upon neighboring tissue.

FIG. 19 depicts the same action as shown for the arterial chutes in FIG. 16 as applied to the veins. More specifically, FIG. 19A is a schematic summarization of venous flow to the donor heart on the left and the innate heart of the recipient on the right, before venous switching in FIG. 19A, and after switching in FIG. 19B. In FIG. 19B, the X strike-out indicates that circulation through the donor heart having been switched from the donor into the circulatory system of the recipient, the native heart has been removed.

FIGS. 20A thru 20C depict the same starting and ending points of the blood diversion chutes at the ends of any one of the arterial bloodlines shown in FIG. 15A, that is, full retraction and full extension, shown in FIGS. 18 and 19 where an intervening position at the center indicates that this is a metered rather than a sudden compound vascular bypass, or sudden switched-organ transplantation. Instead of suddenly shifting from the fully retracted to the fully extended positions, movement of the blood diversion chutes between these endpoints is gradual, implanted sensors alerting the implanted controller as to the extent of antibody release.

The chute driving servomotors such as that shown in FIG. 9 can advance or retract the chutes at any rate to any point along the trajectory separating complete retraction to complete extension. More specifically, FIG. 20A shows the action of the arterial blood flow diversion chutes in a metered switch organ transplant wherein each servomotor moves its respective flow diversion chute between the fully retracted position in FIG. 20A, through the intermediate position in FIG. 20B, to the fully extended position in FIG. 20C at which point the graft organ has been transferred into the circulatory system of the recipient.

In this way, the process effectively ‘feels’ and ‘inches’ its way to full extension at which blood flow is entirely through the donor organ. Were this a metered double heart transplant, the endpoint would be closer to if not at the state depicted in FIG. 20B at which the blood of donor and recipient would eventually represent a 50/50 mix. The extent to which the process and therefore the proportion of donor blood in the mix depends when the chutes can remain at the more central endpoint with no more than manageable disturbance induced. In both sudden and metered switch transplants, the implanted disorder response system, druglines, and jacket accessory channels into which these feed remain for postoperative therapy. If donor and recipient stumps are anastomosed, the bloodlines or mainlines can be removed.

FIGS. 21A thru 21C shows the same three stages in a metered switch organ transplant, here the heart, for each of the veins shown in FIG. 16 as does FIGS. 20A thru 20C show for the arteries. In a metered compound vascular bypass solid organ transplant, the advancement of the diversion chutes, hence, the reative proportion of donor and recipient blood flowing through the graft organ, is continuously adjusted by the master control microprocessor on the basis of sensor feedback that would immediately indicate the intensity of the immune response.

More specifically, FIG. 21A shows the action of the venous blood flow diversion chutes in a metered switch organ transplant wherein each servomotor moves its flow diversion chute between the fully retracted position shown in FIG. 21A, through the intermediate position shown in FIG. 21B, to the fully extended position shown in FIG. 21C. FIG. 21B shows the arterial blood flow diversion chutes in a metered switch organ transplant at the intermediate position where reciprocal cross-circulation exchanges the maximum volume of donor and recipient blood between the two. FIG. 21C to the fully extended position in FIG. 20C at which point the graft organ has been transferred into the circulatory system of the recipient.

The master control microprocessor immediately applies this information to the positioning of the diversion chutes to halt, reverse, reverse and then continue forward, or adjust in rate of advancement or retraction while at the same time coordinating this adjustment in the proportion of donor and recipient blood through the graft organ by releasing medication to counteract any adverse reaction. This hunting action continues until the excessively adverse reaction has been overcome.

FIGS. 22A and 22B show the changes in the connections of the bloodlines during resituation of the donor organ from the circulatory system of the donor into that of the recipient, that is, before the graft organ has been harvested in FIG. 22A and after it has been placed in the recipient in FIG. 22B. The replacement of a lung is purely exemplary. Transplantation of a lung requires connection of the pulmonary arteries and pulmonary veins, schematically represented here as summarized. Lungs are orthotopically, not heterotopically transplanted. The practicability much less necessity for the heterotopic transplantation of a lung are the least of any major organ.

In FIGS. 22A and 22B, part numbers are consistent with those in the other drawing figures, so that 8 is a schematically depicted arterial drugline, while 8′ is a venous drugline, all druglines passing into an accessory channel inside the valve-jackets, 9 is a summary bloodline representing the blood supply, 9′ the same representing the venous drainage, and 41 is a flow or stream line showing the direction of flow. The condition depicted in FIG. 22B offers different options in addition to allowing the condition shown to remain so that blood flow can be switched between the native vessels and either or both arterial bypass 49 or venous bypass 49′.

Determined on the basis of clinical judgment, anastomosis of either or both stump junctions along suture lines that arterial 46 and that venous 46′ can be omitted and the bypass left in place indefinitely as a prosthesis. One consideration in this decision is that a later transluminal, or transcatheteric, procedure will have to pass through the prosthetic bypass rather than through the native structure. If the drugline 8 or internal configuration of the diversion jacket or valve prohibits transluminal treatment, the procedure is endoscopic. Because all basic side-entry jackets, diversion jackets, and valves incorporate at least one accessory channel to directly pipeline target drugs to the treatment site, the need for a later transluminal intervention is slight.

Hypothetically then, should the donor heart in a heterotopic double heart switch transplant require the removal of obstructive plaque, for example, a coronary artery bypass graft, preferably performed endoscopically rather than a percutaneous transluminal, or transcatheteric, angioplasty is performed, the surgical procedure long established as more durable than that transluminal. When donor and recipient stumps are anastomosed as shown at 46 and 46′ respectively, either or both arterial bypass 49 and venous bypass 49′ can be removed.

Ordinarily, the stumps are anastomosed and only recipient arterial diversion valve 48 and donor venous diversion valve 50 left in place in order to retain their respective accessory channels upstream to anastomotic suture lines 46 for continued therapy as necessary. Periodic imaging should supplement implanted sensor feedback to the drug regulating implanted microcontroller, or more likely microprocessor, which controls the drug reservoir outlet pumps or drip stopcocks. Accordingly, every case unique and different vessels differently affected, arterial and venous bypasses would normally be treated alike, but to accommodate different conditions of disease or injury, either can deviate in any of the foregoing particulars.

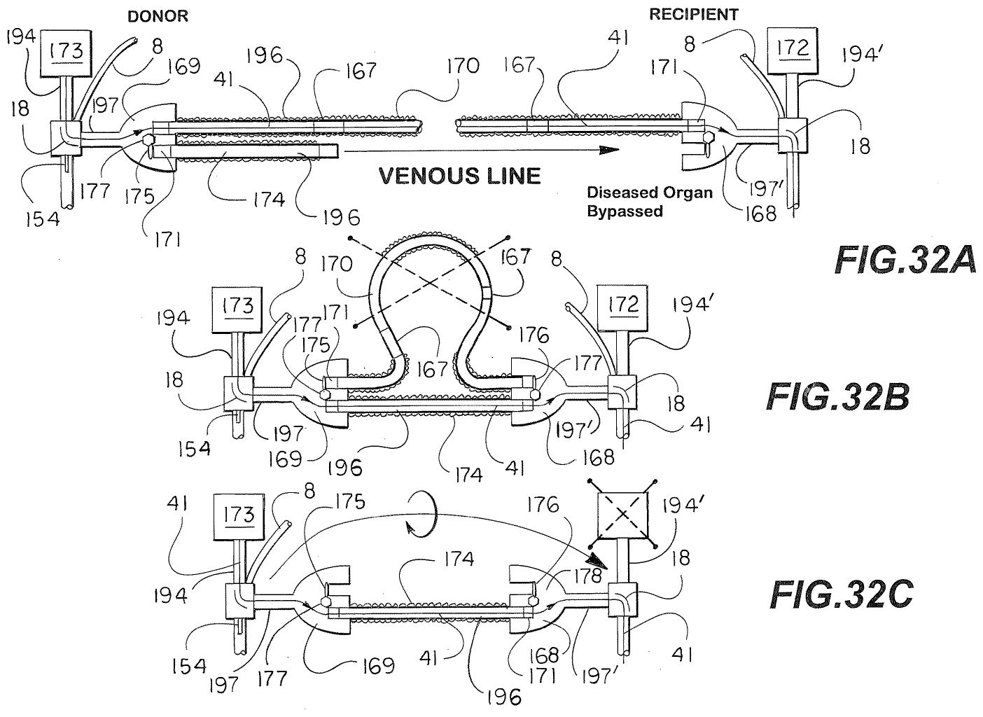

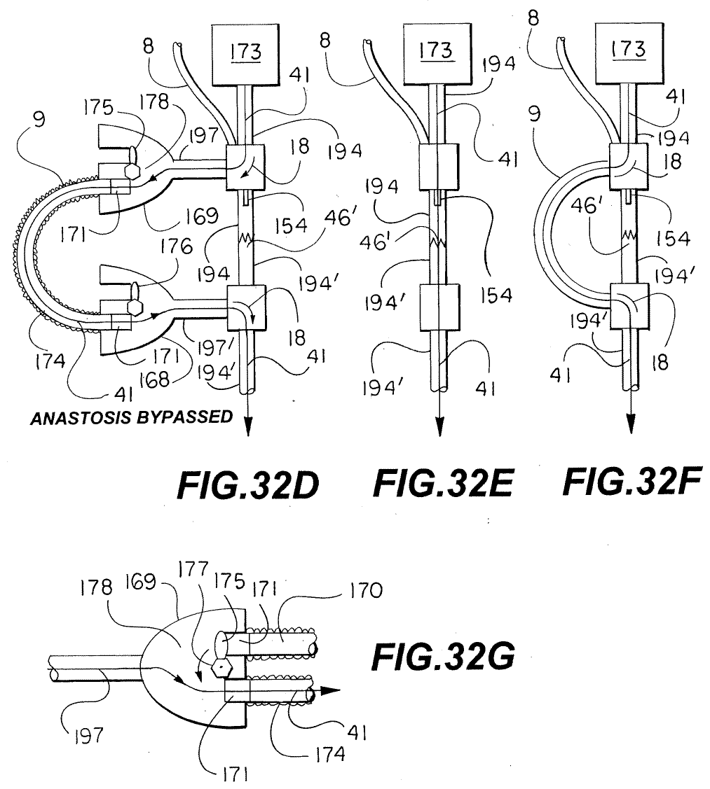

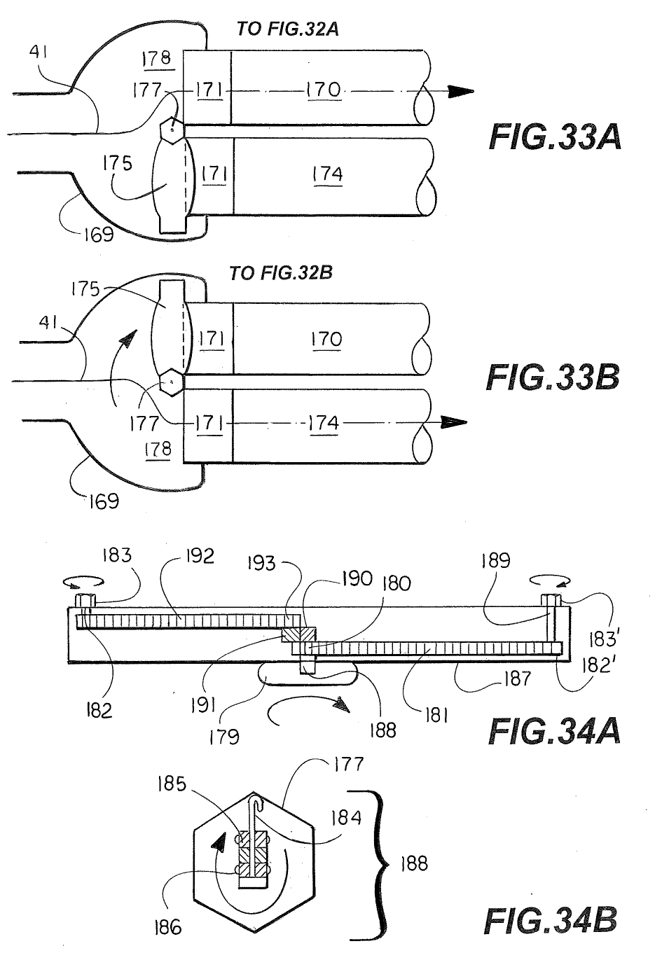

Bloodlines, or mainlines, summarized as part numbers 9 and 9′, must span the distance between the donor and the recipient over a length that may result in these being too long to position in the recipient so as not to encroach upon neighboring tissue, as well as necessitate more pressure to traverse these spans. Once the graft organ has been placed in the recipient, mainlines, here bloodlines 9 and 9′ and druglines 8 continuous with the accessory channels which course through the valves, can be reduced in initial diameter and length to that appropriate in a number of ways. These include:
1. Use of the expansion structure described above in section 2b(2)(f), entitled Expandable Structure to Accommodate Growth and Self-adjust in Size in Vascular Segment Prostheses, Lines, and Confluence Conduits Placed in Children, provides an end-tensile strength or failure strength or bursting force sufficient to withstand a forceful direct impact by a blunt object and if not irreversibly deformed, spontaneously return to the dimensions required.
2. The use of an individual bloodline switch device as described in section 2b(1)(b)(b13), entitled Bloodline and Drugline Switch Devices: Long when Intercorporeal to Short when Intracorporeal, or the Reverse when Intracorporeal to Accommodate Growth, illustrated in FIG. 32A thru 32G.
3. The prosthesis manually pulled to its adult length, additional shortening is obtained by cutting off a segment from either or both ends of the prosthesis, bloodlines 9 and 9′ and druglines 8 by shortened in the same way. The blood passing through the prosthesis little diverted into the reentrants posed by the entry from the lumen proper into the vaults, the effective internal diameter of the prosthesis is little affected, and if necessary, any accumulation of clot is removed by using druglines 8 to release an intermittent low dose heparin drip.
4. Where the distance separating donor and recipient allows, tubing that incorporates successive thicker and thinner walled sections allows involuting (telescoping, invaginating) the thinner into the thicker sections. This reduces the internal diameter of bloodlines 9 and 9′ and druglines 8, which if necessary, is easily avoided by compensating for the reduction through the use of tubing equally larger in internal diameter at the outset. Use of this method assumes that the successive telescoped sections are sufficiently pliant as not to irritate neighboring tissue.
5. Bloodlines 9 and 9′ and druglines 8, are interposed by a bridge section connected at donor and recipient ends by diversion jackets. Once the graft organ has been placed in the recipient, facing ends of bloodlines 9 and 9′ and druglines 8 are ‘anastomosed’ with strongly adherent surgical grade tape and the diversion jackets with bypass tubing removed.

Larger distances separating transplantation donors and recipients are accomplished with bloodline-switches as addressed below under Description of the Preferred Embodiments of the Invention, subsection entitled Long-to-Short Bloodline-switch Device.

While a protective lung cage and the removal of an adequate length of gut would allow positioning a lung in the abdominal cavity, air delivered to the lung through a trachea extended with synthetic tubing at the correct pressure would likely require the additional implantation of a small assist air pump, pressure sensor, and oximeter sensors. However, because the blood supply and drainage afforded by switch transplantation assure virtually normal oxygenation and removal of carbon dioxide, hypoxia in any lobe should not arise.

Such an arrangement could be made to work, but the need for it would appear rare, mostly due to chest injuries that irreversibly damaged both lungs where the use of venovenous extracorporeal membrane oxygenation, for example, could sustain the patient for more than a short time (see, for example, Vyas. A., and Bishop, M. A. 2022. “Extracorporeal Membrane Oxygenation in Adults,” Online, Treasure Island, Florida: StatPearls Publishing Co.; Tellas, I., Brochard, L. J., Gattarello, S., Wunsch, H., Junhasavasdikul. D., and 6 others 2022. “The Physiological Underpinnings of Life-saving Respiratory Support,” Online, Intensive Care Medicine 12:1-13; Pham, T., Brochard, L. J., and Slutsky, A. S. 2017. “Mechanical Ventilation: State of the Art,” Online, Mayo Clinic Proceedings 92(9 (: 1382-1400; Makdisi, G. and Wang I. 2015. “Extra Corporeal Membrane Oxygenation (ECMO) Review of a Lifesaving Technology,” Journal of Thoracic Disease 7(7): E166-E176; Hill, J. D., O'Brien, T. G., Murray, J. J., Dontigny, L., Bramson, M. L., Osbom, J. J., and Gerbode, F. 1972. “Prolonged Extracorporeal Oxygenation for Acute Post-traumatic Respiratory Failure (Shock-lung Syndrome). Use of the Bramson Membrane Lung,” New England Journal of Medicine 286(12): 629-634). Lung disease and related defects often extending to the trachea and bronchi, when diseased or defective as to justify its replacement, the trachea and much of the bronchus, ordinarily not included in the transplant, is included as shown.

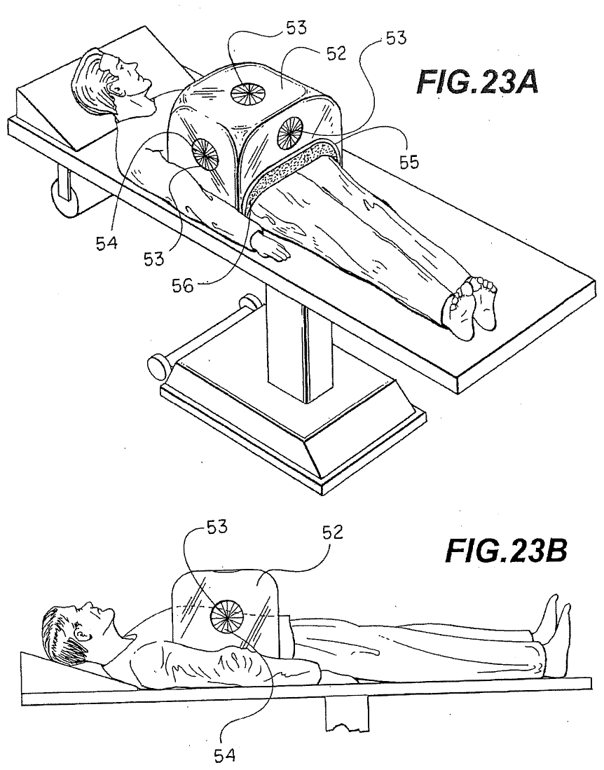

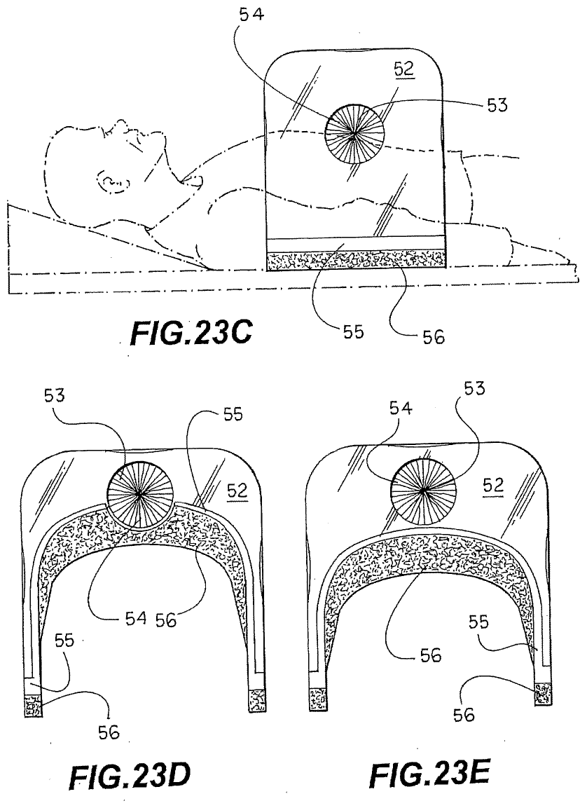

FIGS. 23A thru 23E provide in FIG. 23A an overall perspectival view of a surgical chest dome as addressed above in section 2b(2)(k) with an overall side view in FIG. 23B, a closer side view showing wraparound cast iron weight 55 and foam cushion 56 running around the bottom, in FIG. 23C, the upper, or head-facing (superior, headward or cephalad, craniad), surface thereof in FIG. 23D, and the lower, or foot-facing (inferior, caudad, footward), surface in FIG. 23E. A surgical chest dome serves to equalize the pressure surrounding the surgically breached chest with that intrapulmonary, thereby averting a surgical pneumothorax. The weak vacuum inside the chest dome will usually require the introduction of oxygen or if necessary, the use of mechanical assistance, preferably through a nasal tube, to allow spontaneous breathing.

Surgical chest dome 52 is applicable to any surgical procedure that requires entry into the chest but was devised to allow spontaneous ventilation, or normal breathing where cardiopulmonary support or extracorporeal membrane oxygenation is not used, under regional if not local anesthesia as preferable in a switch transplant. Cardiopulmonary support and/or general anesthesia often leave the patient with cognitive deficits, and a mechanical ventilator operated by anyone not expert in its use can do serious damage to the alveoli in the lungs.

If air enters the dome, the response of the vacuum pump with limit switch is to instantaneously equalize the pressure within to that outside the dome. When the graft organ, such as a heart, is to be transplanted, the organ is harvested and placed within the dome with bloodlines and druglines attached before the chest of the patient is opened and dome 52 placed over the patient. Alternatively, a larger rectangular glove opening is provided to allow such a larger object to be passed through. In FIG. 23A surgical chest dome 52 is made of a clear transparent plastic such as polycarbonate. Glove openings 53 on each side of dome 52 give the operator and assistants easy access to the operative field. Glove openings 53 are filled by stiff bristles several layers deep 54 which brush against the sides of the gloves and any objects introduced or removed to leave virtually no space through which air might enter.

Should air enter nevertheless, the vacuum pump with limit switch will immediately reinstate the rarified condition inside dome 52. Further to assure an airtight fit of dome 52 to the surface of the operating table and/or any bedding or covering, weight 55 runs around the dome 52 just above foam cushion 56 along the bottom periphery. For rust resistance, cast iron dome surround 55 toward the bottom of the dome is galvanized with 0.001- to 0.003-inch-thick inorganic zinc anti rust spray, then top coated with a rust resistant epoxy, phenolic, acrylic, or silicone paint, for example. When the graft organ is placed beside or on the patient before dome 52 is set down, small segments of foam lining 56 if not sufficiently compliant as not to compress the bloodlines are replaced with a more compliant foam.

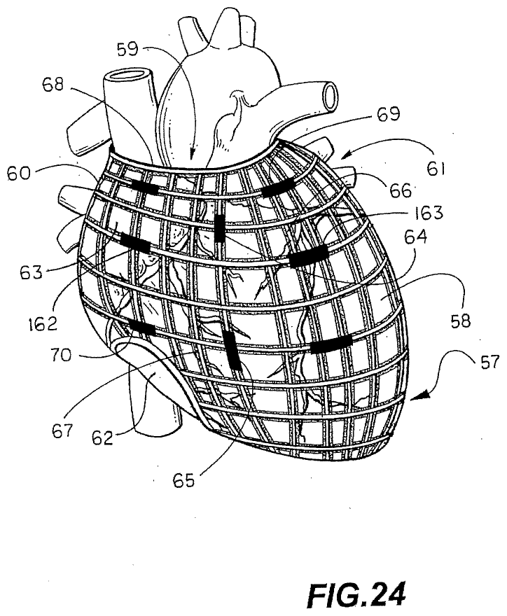

FIG. 24 shows a heart cage (grid, grating) for surrounding a heterotopically bypass, or sudden switched-organ, transplanted heart when placed in the lower abdomen, for example, where adequate protection by surrounding bone is lacking. To minimize its weight, the cage is fabricated from narrow hollow titanium or stainless steel tubing. FIG. 24 provides an anterior (frontal, face-on) view of a heart cage. The cage, fabricated by electrical resistance (‘spot,’ electrical welding, arc welding), comprises two complementary sides, each conformant to the shape (profile, contour) on its respective side of the heart, always asymmetrical, to be enveloped and complementary to the periphery of the opposite half. The halves are hinged at the back by electrically welded hinges (not shown) made of the same titanium or stainless steel as the grid.

For strength and to avoid current flow, the weld metal is the same as the grid. The halves come flush together at the front, where spring-loaded upper latch-type lock fastener 64 and that lower 65, likewise attached to the grid frame and made of the same material, allow the cage to be opened should a follow-up procedure, such as examination by a transluminally passed intravascular ultrasound probe, or a coronary artery bypass graft become necessary. All of the hinges to include those along the back, or posterior, junction where the sides come flush together or seam and spring-loaded latch fastened locks similarly positioned along the front, or anterior, seam are made of the same titanium or stainless steel, each electrically welded to the grid about a third of the distance from the top and bottom.

The cage comprises gridwork 57 of hollow titanium or stainless steel tubing 3/16 inch or ½ centimeter in outer diameter, with a tubing wall thickness of 1/16 inch and grid apertures (holes, openings) 58 of 1 inch or 2-½ centimeters, following the same outer shape (profile, contour) as the contained heart itself. The grid stands off at a distance of about ½ inch or 1-¼ centimeters from the heart per se, affording clearance for the interposition of a protective layer of perforated viscoelastic polyurethane foam, bonded along the interior surface of the cage grid tubing and provides an opening at the top 59 to allow the superior vena cava, aortic arch, and its vessels with any perivascular fat, vasa vasorum, and vasa nervorum to pass through without contacting the sides of the foam-lined top opening.

The grid also has a side opening 60 at the upper right and one at the upper left 61 to allow the pulmonary arteries and veins with supportive nervelets and fine vessels pass through. Toward the bottom on the right is another opening 62 to pass through the inferior vena cava with sufficient clearance for its supportive nervelets and fine vessels to disallow encroachment by the margins of the opening. Applied after reciprocal cross-circulation through the mainlines connecting the donor and recipient to effectuate the metered bypass, or switched-organ transplant, has already been instituted, which must not be interrupted, the heart cage requires not only permanent openings 59, 60, 61, and 62 but must continue with openable flap sections respective of each, opening flap 63, spring-loaded hinged at 68, lift flap 66, spring-loaded hinged at 69, and flap 67, spring-loaded hinged at 70.

Opening flaps 63, 66, and 67 lock in the closed position are preferably kept closed as indicated by spring-loaded hinges but can also use the same type spring-loaded latch engaging fasteners as those at the anterior midline to remain secure when not lifted to allow this clearance. Such an optional fastener is shown on the heart right side as part number 162 and on the heart left side as part number 163. The grid tubing must be able to withstand deformation as the result of a direct blow and at the same time, allow the operator to intentionally introduce bends as might be necessary to fit a less than ideal graft heart that had disproportionately adapted to its impairment as to present an anomalous contour. This is accomplished by heating the grid where the bends are needed with a small hand-held propane blow torch, for example, rendering the areas to be altered pliant.

Accompanied and assisted by another heart, supported by medication immediately dispensed automatically upon detection of the need therefor, and removed from the toxic condition in its host, the donor as well as the native heart should experience a significant measure of relief and recovery, even if that native had undergone heart failure. Only a significant alteration in contour after placement warrants its removal for remolding. The heart cage incorporates a sufficient number of flexible points to facilitate its removal if needed.

In use, the cage is opened, and the donor heart, having been mobilized, that is, freed of any attachments or adhesions, especially to its rear, with diversion valves, drug, and bloodlines attached to the vascular stumps, is set down on the foam lining the grid. Care is given to assure that vessel stumps rising upward clear top opening 59 without contact other than lightly against the foam lining opening 59 and pulmonary vessel stumps at the upper sides pass through side openings 60 with associated clearance flap opening 63 and opening 61 with associated clearance flap 66, and opening 62 with associated clearance flap 67 without metal contact. The cage is then closed around the heart and locked closed by means of spring-loaded latch fasteners along the front seam where right and left halves come together.

### Carotid Bypass and Prosthesis

FIGS. 25A and 25B show a right-sided carotid bypass device which allows an ischemia-free carotid endarterectomy, and which, if necessary, can remain in position as a prosthesis indefinitely. Supported by druglines 8 and drug release pore 31 beneath the distal tip of diversion chute 18, a concentrated statin drip, for example, can be directly targeted into the carotid to take advantage of its topical or pleiotropic healing properties in addition to a systemic dose, reducing any risk of inducing diabetes. As pertains to the other figures, the blood diversion servovalves are made to appear somewhat larger than actual, and the angle of each with respect to the carotid is that which best accommodates the device.

When incorporating separately controllable servovalves that allow continuous variability in the degree of diversion chute projection into the lumen, the device can detour all or a portion of the blood around either branch of the native carotid to expedite healing and/or allow open examination and further treatment. In such diversion, the sum of blood passed craniad is that normally provided by the carotids. When equipped with separately controllable highly damped solenoid driven valves such as shown in FIGS. 7 and 8, actually functioning as flow switches, all or none of the flow normally passing through the carotids is controlled.

Complete closure of the lower valve causes all blood flow to pass up through the bypass, while the valves at the center and at the top control flow through either branch of the common carotid. When either or both branches of the common carotid have been irreparably damaged by disease or the need to resect tumors, for example, the device can be left in place permanently as a prosthesis, with the immediate diagnosis and treatment of erratic fluctuations in blood pressure, blood gases, and blood glucose applied by the implanted disorder response system. These mid- and postprocedural problems associated with the loss due to surgical resection or trauma of the carotid bulbs where such a prosthesis without the support of an implanted automatic disorder response system is not used are briefly addressed above in Background of the Invention.

In FIG. 25, of the branches of the right external carotid artery included in the figure, ST is the superior thyroid artery, L the lingual artery, F the facial artery, M the maxillary artery, and SupT the superior temporal artery. Part number 88 is the servovalve attached to the common carotid, 89 that attached to the external carotid, and 90 that connected to the internal carotid. The orientation of the three valves usually required to perform a carotid endarterectomy or the two in the infrequent circumstance that the external carotid is unaffected, is that least encroaching upon neighboring tissue and minimally if at all presenting a cosmetically conspicuous bulging of the neck.

The edges and corners of the valves are rounded to eliminate abrasive or gouging of neighboring tissue, and the valves can be wrapped within a soft sock to eliminate any irritation. Accordingly, the three valves shown in FIG. 25A have been drawn in the plane of the figure for visual clarity and must not suggest such a disposition is prescriptive. Each servovalve is shown with a direct manual fine control assist device detachably connected to its underside in a separate enclosure which will be removed before the patient is closed. The structure of the direct manual fine control device is delineated above and shown in FIGS. 10A thru 10C. Valve 89 is positioned just above the bifurcation of the external carotid and facial arteries to minimize is not eliminate the blind pocket, or cecum, wherein clot would accumulate were it positioned at a higher level.

Valve 88 on the common carotid must remain sufficiently open, or retracted, to allow blood to pass into the facial, lingual and superior thyroid arteries. While it might appear that the internal carotid has been shown disproportionately wider than the external carotid, the illustrations provided in the popular anatomy textbooks such as Cunningham, Morris, Gray, Grant, and Sobotta, were drawn from cadaver preparations, not surgical patients in whom the blood under pressure produces distention (dilatation, dilation) as shown. The common and internal carotids are large enough in caliber that in order to minimize their size and weight, the lumina of the vascular servovalves connected to these are made smaller. The rationale for this reduction without adverse sequelae is provided above under Background of the Invention.

### Body Surface Ports

Body surface ports are addressed above in section 2a, entitled Background General, and the placement of these addressed above in the subsection entitled Implantation of Body Surface Ports. This application concerned with vascular valves and servovalves, only a condensed summary and simplified description of body surface (on-skin, epicutaneous, extracutaneous), subsurface (subcutaneous subdermal), and ports which combine opening on and beneath the skin for use with an implanted automatic disorder response system can be reviewed.

Body surface ports can incorporate power button cells, which stored above-skin can be replaced, or beneath skin recharged transdermally, or transcutaneously, mechanical control knobs such as to extend or retract a push/pull, or Bowden, cable, for example, in urological applications as will be delineated below; electrical button switches to actuate tiny lamps for illumination and implanted electrostimulators when not triggered automatically by sensor feedback to the system control microprocessor; and locked system override switches which the clinician can unlock if for any reason such becomes necessary.

Subsurface (subcutaneous, subdermal) body ports for use with an implanted automatic disorder response system are portacaths, or mediports, such as those in common use, which have been modified to incorporate multiple entry openings to allow the simultaneous replenishment of subcutaneously, or subdermally, positioned small flat reservoirs storing drugs in fluid form as well as any of the manual controls listed above. As is true with a conventional single entry portacath, when drug delivery passive due to gravity and the opening is above-skin, that is, at the exterior rather than subcutaneous, the way is clear for diagnostic use, such as the drawing of blood or delivery directly to the pipeline targeted organ, gland, or original root-site of disease, for example, of fine transluminal diagnostic devices, such as an angioscope, and therapeutic devices such as an excimer laser.

In summary, such ports fall into three groups, those nonurological which are usually positioned subcutaneously in the pectoral region, those urological which are usually positioned to a side of the mons or mons veneris, and those mixed which incorporate both, wherein the medicinal openings are still subcutaneous. Both for reasons of comfort and cosmetic, in all cases, a central object is to achieve a small shape factor with minimal weight. When drug delivery is not through a passive gravity drip but rather directed by the microcontroller—or in more complex comorbid disease, a microprocessor—the reservoir and its outlet pump or drip stopcock obstruct such passage.

For this reason, the attempt is made to always include a gravity fed line. In general, only urological ports provide an opening to the exterior for connection of a tube to pass ursine into a collection bag. Such ports will often include electrical switches to control and one or more button cell batteries to power electrical components, and control knobs mechanical Bowden cable of electrical, for example, in addition to subcutaneously positioned inlet openings for injecting drugs. Otherwise, urine effluent line 110 from the bladder or neobladder is always free of obstructions.

As shown in FIG. 26B, drugline K (small square second from the right toward the bottom of the figure) to either or both kidneys, such a drugline omits components, here the drug reservoir 102 and the drug outlet pump or drip stopcock 103 which would block the path of the cabled device to be employed. The use of a cabled device would thus necessitate a second K line without the obstructions posed by 102 and 103. Generally, ports incorporate an above-skin opening and omit the reservoir 102 and pump 103 only when diagnostic devices will be used or biopsy samples drawn, other openings for the delivery of medication located beneath the skin. An effluent line such as 110 must come to the body surface.

The arrangement shown in FIG. 30 not a prosthesis as that shown in FIG. 28, is meant for a patient such as a public performer suffering from urinary incontinence and/or overactive bladder frequency. In such a patient with retained refractory urge and urinary tract sensation not ameliorated by botulinum toxin injection, bladder stretching, or drugs such as mirabegron or solifenacin and lacking control, the system used is of the type shown in FIG. 30 where a port of the kind shown in FIG. 26C provides controls for the user to switch between unilateral or bilateral partial or total automatic or voluntary evacuation, hence, the rate of collection.

By apportioning the collection of urine between the native bladder and the neobladder, the patient can reduce the frequency of having to flush through the passageway comprising lines 144 and 144′, neobladder 145, outlet pipe 110, and surface port 147 with a disinfectant. A port preferably positioned to a side of mons pubis having an above-skin central outlet pipe to pass urine into a urine collection bag tethered about the ipsilateral thigh and surrounding subcutaneous drug injection openings is shown in FIG. 26C.

In contrast, the prosthesis shown in FIG. 28 is for a patient having undergone a pelvic evisceration or one having been severely injured so that urge sensation has been lost. The system is therefore devised to collect urine of which the accumulated volume will be continuously apparent from the weight of collection bag 148 cinched about a thigh. To evade notice, the collection bag used is a little shorter than the thigh and flat like a hot water bottle. Not shown in FIG. 30, the collection bag empties through an outlet hose at its bottom with a removable screw cap to allow the bag to be emptied into a bathroom receptacle out of the sight of others.

While such a device would never be placed for the capability alone, once implanted, access to the urinary system is fundamentally facilitated. The renal arteries, for example, can be directly accessed through ductus side-entry jackets and the parenchymata (parenchymas) through nonjacketing side-entry connectors. Approach from above is through a body surface port such as that shown in FIG. 26B, in the pectoral region, while access from below is through urine outflow pipe 110 into the synthetic parts, and into the native parts through the portacath-type entry holes 107.

Access to the urinary tract from above through a body surface port in the pectoral region has already been addressed in terms of affording a urologist endoscopic, laser, electrosurgical probe, or any other cabled device or medicinal access to the urinary tract from above. That different instruments will likely require the interior passage through the jacket or valve to be configured to expedite passage is superfluous. In FIG. 28, an endoscope introduced through urine outlet pipe 110 can be passed up through neobladder 145 through either neoureter 144 or 144′ thence into the ipsilateral native ureter and kidney.

In FIG. 30, a cabled device can follow the same path as specified for FIG. 28. A distinction pertinent to FIG. 30 which is not a prosthesis so that the native ureters remain intact and are usable whenever the patient does not require diversion into neobladder 145 is that side-entry jackets 144 and 144′, devised to be adjusted switch the outflow of urine between the native urinary tract and the apparatus, allows the passage taken by a cabled device to be controlled in the same way. The native ureters in FIG. 30 where the urinary tract is intact are accessible to a cabled device such as a cystoscope in a conventional manner.

In FIG. 28 where the native lower urinary tract has been removed, the native ureters have been tied off. Nevertheless, in a patient considered to be susceptible to disease in the portion of the ureters below side-entry jackets 144 and 144′ the use of adjustable side-entry jackets ab initio would allow access as with the system shown in FIG. 30. Cabled devices are best passed through passageways that are sterile, and with respect to urine outlet pipe 110, even normal urine contains bacteria. That urine is infected is apparent from a urinalysis (leukocytes, hematuria) as well as the symptoms reported by the patient.

Infected or not when the system is implanted, effluent (outflow, emptying) pipe 110 will remain exposed to contamination and the formation of a tenacious biofilm that will demand strong antiseptic treatment to destroy. As delineated above, pathways affording access to outlet pipe 110 from above is through druglines 8 and 8′ which are large enough in internal diameter to pass through the cabled device. Access from above to the renal arteries and parenchymas is addressed above. An additional surface port opening such of the kind shown as K (leading to a kidney) in FIG. 26B is an option. Access from below can be through urine outlet pipe 110 or one of the holes in port 147 provided for injections.

Access includes the obtaining of blood and biopsy samples. Blood is obtained by passing the collection probe through a side-entry jacket or valve on the target blood vessel, whereas a biopsy is obtained by passing the biopsy needle, punch, or forceps along the pathways specified. The passageway is disinfected first. Note that in FIG. 26B, drug reservoir 102 and outlet control 103 have been omitted from to-the-kidney line K to provide a clear line for endoscopy for example. A second K line can include these components in obstructive form or can provide components that will not interfere with—passage to the kidney. That separate K lines can be provided to either kidney is considered obvious.

When frequent urination is not a problem, bladder effluent line 110 from the bladder or neobladder can be sanitized by releasing an antimicrobial through the accessory channel of the nonjacketing side-entry connected positioned along the bottom of the bladder or ductus side-entry jacket on the ureter and/or an antimicrobial swab run up through bladder effluent line 110 just before use. Any type port can incorporate such above-skin openings and/or components, but for better protection against infection, drug injection openings are best subcutaneous. Subcutaneous drugline entry openings also eliminate the need to remove a cap before injection, thus facilitating quick drug replenishment when all drugline openings can be accessed simultaneously with a multiple needle or needle free disposable cartridge jet injector nozzle injection head such as those depicted in FIG. 27.

That urine outlet bladder effluent pipe 110 opening and any other above-skin opening to the exterior-always covered over by a protective cap and structured to retain antimicrobials—can be used in the reverse direction to pass diagnostic tools for examining the interior of the bladder, ureters, and renal pelves on a permanent basis without the need for a ‘keyhole’ incision each time or a bandaged over, readily infected wound to allow periodic reexamination should be evident, as should the option to remove the device once a temporary condition clears.

FIG. 26A shows a vertical left side sectional view of a nonurological (that is, without an above-skin urine outlet) subcutaneous port with multiple openings for injecting drugs, FIG. 26B providing a schematized depiction of the same port in the context of an implanted automatic disorder response system. If few, the drugs are injected sequentially by hypodermic syringes or alternative means of injection 101. To minimize the duration and imitation of injection, more than two drugs are preferably injected simultaneously with a multiple needle or multiple nozzle needle free disposable cartridge jet injector head such as that depicted in FIG. 27.

Covering a 2 centimeter or so circular area of skin prepared by electrolytically removing the hair over the area, urological ports must provide an underlying surface treatment such that the interface with the skin will not result in infection or irritation such as itching. The port removable properly only by a trained clinician, the wearer can accomplish sanitizing and inflammation reversal without removing the port by using an eye dropper to drip a back pad of gauze placed before the port is attached with an antimicrobial such as an alcohol or hydrogen peroxide and/or an anti-inflammatory such as dilute prednisolone or a fluid preparation of triamcinolone provided in a vial small enough to prevent misapplication.

New materials which incorporate antimicrobials and anti-inflammatoires are briefly addressed above under the section entitled Background of the Invention. Drug injection openings 107 in medicinal ports serve as the points of entry into the sidelines, thence through accessory channels 8 of the side-entry connectors, jackets, and valves at the target tissue or vessel and to prevent their disconnection are fused to the drugline 8 into which each flows. Multiple self-sealing membranes, or septa, 121 are shown as covering each port opening separately but can comprise one continuous membrane, or septum. Self-sealing membranes 121 are absorbent and wetted with an antimicrobial at the same time with a syringe introduced into injection openings 107 by inserting the Huber injection needle to just enter openings 107.

Druglines 8 are subcutaneously tunneled to the level of the target organ, gland, original root-site of disease, or tissue and then routed along the course to the target least like to strangulate intervening tissue. In FIG. 26C, the urological port is positioned to a side of the mons pubis or mons veneris so that druglines 8 must move upwards. Accordingly, in the lower position of FIG. 26C, drugline 8 would actually proceed craniad or as nearly so as would preclude kinking upon bodily movement, for example, directly upon exiting from drug injection opening 107.

As shown in FIG. 26C, were it necessary to move downward, would turn around while subcutaneous into the upward, or craniad, direction. Highly dilute (low viscosity, watery) injectants can be delivered directly to the target tissue by gravity. In FIG. 26B, where to allow direct gravity feed, the drug delivery line (drugline) 8 leading into an accessory channel of a nonjacketing side-entry connector on the organ fibrosa or into that of a side-entry perivascular jacket or servovalve on the renal artery, for example, to the kidney, denoted by the K, is continuous, that is, omits a drug storage reservoir 102 and reservoir outlet pump or drip stopcock 103. If the line is ordinarily used for higher viscosity substances, a reservoir 101 and pump 102 are present and actuated as needed manually.

Higher viscosity drugs are injected into reservoirs such as those marked H for the heart, L for the liver, and B for the brain. The size of any component in FIG. 26B, such as the drug reservoirs 1, 2, and 3 generically identified as 102 or drug reservoir outlet pumps or drip stopcocks 103 will vary with the dose and dose release schedule of the substance injected, and the length of druglines 8 will vary based upon the distance from the body surface port 100 backplate as shown in FIG. 26C to the target side-entry component. Replenishment of the flat vial storage reservoirs 102 for the independent release of each drug to a different organ, gland, or root root, or site, of disease to treat comorbid disease under the morbidity-distinguished hierarchical control channels or branches executed and coordinated by the system controlling microcontroller or microprocessor according to its prescription-program.

As in a conventional portacath, or mediport, migration is prevented by passing suture through small loops as shown or holes 104 or about the periphery of the port, and migration of the lines feeding into the accessory channels 8 by omitting joints with the potential to become disconnected. Shown in FIG. 26B, the pumps at the outlets of drug reservoirs 1, 2, and 3 are controlled through wires 105 by the fully implanted automatic control system microcontroller or microprocessor 106. If for any reason hard wiring should be avoided, the commands are issued by controller 106 by radio remote control, typically Bluetooth, power replenishment by transcutaneous energy transfer, the frequencies employed cautiously chosen to avoid extrinsic actuation by garage door openers, for example.

FIG. 26C shows a vertical cross-sectional side view of a port which combines an above-skin urine outlet 110 from the bladder or neobladder and subcutaneous drug injection openings 107. In contrast to nonurological ports which are positioned subcutaneously in the pectoral region, urological ports are preferably positioned to a side of the mons pubis or mons veneris where these are easily seen and used by the patient, whether to connect a urine drain hose to or disconnect it from the urine outlet opening, adjust a ureteral valve, or make less awkward the syringe-injection of a drug into an above skin drugline opening by the patient.

Accordingly urological ports must be provided with a. Covering components to prevent microbial intrusion, and b. Situated inferior to the target tissue where passive gravity feed is not possible, a small pump, typically peristaltic. To assure freedom from infection, urine outlet, or effluent, line, or tube 110 is closed off by airtight screw-on or press-to-engage detent cap 108, and the cap filled with a small gauze wad 109 wetted with a potent antimicrobial.

Thus, should it become contaminated, the opening into urine outlet, or effluent, line 110 is routinely sterilized. The diversity of implanted components that might be controlled from such a port is considerable, and as commented upon above, includes neuromodulatory devices such as electrostimulators, control knobs the wearer uses to switch urine outflow from that normal into a proper receptacle to effluence internally through the port into a collection bag, prepositioned diagnostic viewing devices, and manual switches to release drugs by the wearer in response to predictable sources of irritation.

FIG. 27 shows the two main means for simultaneously replenishing the drug reservoirs implanted with an automatic disorder response system. The main object in simultaneous replenishment is to expedite this process with patients having multiple comorbidities who whether because of cognitive impairment due to dementia or infancy are prone to become frustrated and unruly when several injections are needed. Another advantage is that the individual syringes or needle free disposable cartridge jet injectors are prepared and confirmed as correct according to the prescription load list well before the procedure, thus effectively eliminating human error.

Autoinjectors such an epinephrine pen are discounted as facilitating use by the patient, which is discouraged; replenishment should last for a period long enough that return to the clinic is not so frequent as to provoke noncompliance. FIG. 27A shows a metal or plastic manual syringe simultaneous injector which allows multiple substantially conventional individual injection syringes 101 in FIGS. 26A and 26C collectively 111, to be used simultaneously. The drawing figures comprehend four syringes, but any number that will allow injection into such a port with multiple openings, typically 2 to 4 centimeter in diameter, and will fit into the frame can be used.

Each syringe 111 will usually contain a single drug; however, drugs otherwise mixable can be combined in any one. Pending the development of switchable, such as rotatable turret mounted port openings or druglines, each opening is immutably continuous with a certain drugline, so that the destination as to organ, tissue, gland, or original root-site of disease, for example, must determine the contents of each syringe 101 in the set thereof 111. A multiple jet injector such as that shown in FIG. 27B can use interchangeable drug refill cartridges and nozzles. Inserted into the openings in the body surface port and not through the skin, the use of hypodermic needles would not disturb or cause discomfort for the patient.

For example, with the openings and druglines permanently associated, unless more than one drugline is led to the same destination, to inject a large volume of a drug intended for the same destination will require the use of an additional syringe to sequentially inject the same opening, and if the same drug is to be pipeline targeted to different organs, glands, tissues or nidi, these must be separated into different syringes, each aligned to the respective port opening for the destination intended. Alternatively, a confluence path between tips of adjacent syringe barrels can be used to allow two or more syringes to empty into the same reservoir. Using syringes having barrels different in diameter and loading different concentrations of the drugs are two ways to adjust the dose.

Otherwise, the set 111 of individual syringes 101 are unconventional in lacking barrel 112 or plunger 113 flanges, and in situating the hypodermic needles 114 with syringe tips (Luer, slip) as close to the periphery of each syringe as necessary so that each needle will align with its respective opening in the port. Hypodermic needles 123 can be of the Huber non-coring type with tips substantially straight, or if preferred, curved but no more than is essential to avoid coring. The barrels are conventionally calibrated with volume markings 115 in cubic centimeters or milliliters. The manual syringe simultaneous injector is comprised of outer rectangular frame 116 made of plastic or metal fabricated of channel, or U-cross-section stock, through which reciprocally slidable complementary n-section internal rectangular frame 117, made of the same or a different material, can slide reciprocally as a track.

The upper member of internal frame 117 must have bonded to its upper side a floor plate 118 upon which the syringes rest covering an area that will assure the stably vertical alignment of the set 111 of individual syringes 101. As illustrated, simultaneous injection of the set of syringes 111 is accomplished by using the fingers to pull down the lower member, or crosspiece 119, of outer stationary rectangular frame 116 toward the lower reciprocally sliding member, or crosspiece 120, of internal frame 117. This drives floor 118 bonded atop the lower member 119 of outer rectangular frame 116 upward, thus forcing plungers 113 upward to eject their contents. Injection is into multiple openings 107 of subcutaneous port 146 in the sectional view of FIG. 26A and 122 in the extracorporeal view of FIGS. 17 and 27C with backside view in FIG. 26B. Injection point indicating tattoo marks on the skin are shown as 124 in FIGS. 17 and 27C.

The markings consist of two tiny tattoo dots, which barely noticeable, can be different in color and lased away if and when the port is removed. The same markings pertain whether the device of injection is that using hypodermic needles as depicted in FIG. 27A or jet injection micronozzles as shown in FIG. 27B. Showing two of the four injection points is sufficient to align all four or more of the hypodermic needle tips or orifices of the hex nozzles of the jet injector. Front views, FIG. 27A conceals two additional hypodermic needles, and FIG. 27B two additional jet injection fill chambers to the rear. In either, provided the outlets are aligned to the injection openings in the subcutaneously implanted port, the drug fill chambers of the injectors to the rear can be of any diameter.

More specifically, the relative drug holding capacities of the syringe and fill chambers to the rear may be the same, the reverse, or different than either of those seen at the front. Nonmanual injection using ports of the Bard PowerPort® (division of Becton Dickinson-C.R. Bard, Covington, Georgia) type allow the subcutaneously implanted drug reservoirs to be replenished at the rate of 5 milliliters per/second at 300 pounds per square inch, further reducing patient annoyance. Unlike an embodiment comprising disposable cartridge jet injectors, the multiple jet injector shown in FIG. 27B is analogous to the hypodermic needle embodiment shown in FIG. 27A in applying a common driving means to the ejection plural plungers.

Means for the injection of drugs essential to support an implanted automatic disorder response system, FIG. 27B omits tiny seals, washers, valves, and screws, for example, the showing of which would be superfluous for those familiar with such devices. In FIG. 27B, 125 is the head assembly, 126 is the injection thumb actuated release button; 127 the spring release or cocking lever; 128 the main power spring, 129 the common shaft for the drug fill chambers; 130 the cocking lever spring that primes main spring 128; 131 the cocking lever hinge; 132 index and middle finger rests; 133 the injector body, 134 the upper seals of the two front fill chambers 135 and 136; 137 the lower seals of the two front fill chambers; 138 and 139 the two front fill chamber 135 and 136 ejection nozzles; and 140 and 141 the ejection outlet orifices.

FIG. 27 C shows an entirely subcutaneous implanted multiport in a baby where the purpose thereof will almost always be other than to support a double heart switch, or bypass transplant as shown in FIG. 17. The entirely subcutaneous port is of the type shown in FIGS. 26A and 26B as 146 and shown as 122 in the extracorporeal view of FIG. 27C, where the on-the-skin, or cutaneous injection point indicator marks appear as 124.

FIG. 28 shows a prosthesis for the automatic elimination of urine in a patient with a missing or severely defective lower urinary tract into a synthetic neobladder for drainage into a paracorporeal collection bag usually cinched about a thigh, or directly into the collection bag. Both vascular valve-implemented urinary prostheses and assist devices can be unilateral, bilateral, or switched-between the two. As a prosthesis, the system requires no voluntary controls for the wearer to turn on or off automatic collection and voiding, for example, as does a partial dependency system, or urinary assist device for a patient with intact urge and urinary tract pain sensation.

Gut not adapted to conduct urine and prone to metaplastic degeneration which can lead to malignancy, the surgical reconstruction of the urinary bladder or a stoma with harvested gut is discouraged. Because it reveals an inherent proclivity toward malignancy, this is pertinent to a lower tract missing following evisceration due to a malignancy that had metastasized to neighboring tissue. A prosthesis, the system uses nonadjustable, that is, permanently set, diversion jackets, which nonadjustable are not properly referred to as valves, such as those shown in FIGS. 2 and 3 or highly damped nonsparking plunger solenoid-driven valves, shown as part number 32 in FIGS. 7 and 8.

While nonadjustable by the patient, post-implantation adjustment by a clinician can be readily accomplished by endoscopic access through a ‘keyhole’ incision. In FIG. 28, diversion jackets 143 and 143′ are of the nonadjustable nonvalve type shown in FIGS. 2, 3, 7, and 8. Nonadjustable in this regard denotes that the patient, who due to the absence or abnormality of the lower urinary tract lacks urge and pain sensation, requires fully automatic control over urinary voiding. Diversion jackets 143 and 143′ tap off, divert, and pass urine, of which 41 and 41′ represent the flowlines, into synthetic prosthetic ureters, or neoureters, 144 and 144,′ which drain into synthetic neobladder, or neoureter confluence chamber 145, shown in greater detail in FIG. 29. While the patient is erect, neobladder 145 drains into collection bag 148 under gravity.

In this urinary prosthesis where urinary tract sensation and/or self-control is missing, control over voiding when the patient is recumbent must be automatic. To this end, as neoureter confluence chamber, or synthetic neobladder 145 fills, a small bar which presses down on a strain gauge (not shown) eventually imposes the threshold level of force for initiating evacuation (emptying), passing current through a small box fan-configured impeller 150 on the floor, or as shown in FIG. 29, suspended at the center of polymeric confluence chamber 145 to drive the urine out through urine effluent line, or outlet pipe 110 which exits the body through the center of combined above-skin, or cutaneous, outlet, with surrounding subcutaneous drug inlet openings such as those shown in the body surface port 147 depicted in FIG. 26C, thence through collection bag connecting hose 149 into collection bag 148.

FIG. 29 provides a more detailed view of the neobladder or neoureter confluence chamber 145 shown in FIGS. 28 and 30. In FIG. 29, small impeller 150, configured much as a box fan, is positioned at the bottom center, or as shown, suspended at the center of confluence chamber 145. Confluence chamber 145 is molded to the same shape as a native bladder and to allow the longest collection time, as large in capacity as does not encroach upon neighboring tissue or present a weight when filled that induces the sensation of a foreign object. Storage in confluence chamber, or neobladder 145 causes less discomfort than loading collection bag 148 at a quicker rate.

Impeller 150 draws electrical power through wire 151 from one or more button cells positioned about the central bladder effluent pipe 110 outlet in the body surface ports as are the drug injection openings 107 shown in FIGS. 26B, 26C, and 28 and the push/pull cable control knobs shown in FIG. 30. Confluence chamber 145 is made of a stretchable, hence, expandable polymer such as thin medium density polyethylene sheet. As shown in FIG. 28, the prosthetic lower urinary tract is not switchable to normal voiding during the daytime or when the user is before the public, for example.

Accordingly, an antiseptic delivered into the ureters through valves 143 and 143′ will be effective through the entire length of the synthetic system and passed into collection bag 148 so that native tissue is not exposed to it, making disinfection through the use of more concentrated antiseptic unobjectionable. Unless the reservoir and outlet pump or pumps or drip stopcocks are positioned within the pelvis, so that the injection openings in the surface port can be incorporated into a port positioned to a side of the mons pubis, an antiseptic, anti-inflammatory, or analgesic, for example, must be injected through a port superior to, that is, above the level of side-entry diversion valves 143 and 143′ to arrive by passive gravity without the need for intrapelvic componentry.

Ordinarily, this will be through a port positioned in the pectoral region as are conventional portacaths or mediports. The user-controllable system shown in FIG. 30 uses drugs in the same manner as the prosthesis shown in FIG. 28 only when the user switches it to urinary diversion. Switching diversion off, then, allows medication introduced through the drug openings, or injection points 107 in the surface valve treats the entire native lower urinary tract. In FIG. 29, small bar 152 positioned atop impeller 150 pushes down a small rod at the center of a strain gauge (unshown). In a standalone urinary application, the strain gauge sends its output directly to the implanted system microcontroller.

Representation of the impeller as centered within the confluence chamber and the strain gauge at the tope center thereof is exemplary. Either can be located together or separately at a number of positions. That is, while shown positioned on top of impeller 150, small bar 152 to push against rod and strain guage can be a button of any shape and situated in any location along the interior surface of confluence chamber 145 except at the top thereof. To assure comfort and allow the confluence chamber 145 to be produced in no more than a few sizes, the controller is programmed to initiate voiding, into a collection bag 148, for example, when the signal strength received from the strain gauge is just short of that at which the user experiences discomfort.

In a system to treat comorbid disease whereof the urinary voiding subsystem constitutes but one level requiring therapeutic monitoring and coordinated response by a hierarchical control system in the treatment of multiple conditions, the strain gauge sends its output through the local or lowest level control channel, meaning that dedicated to the urinary subsystem, whence the signal rise up through and is processed by increasingly higher levels of control in the hierarchy to the multiple implant system master controller, here, a microprocessor.

FIG. 30 provides a schematic anterior view of a bilateral automatic urine collection and voiding system for a patient retaining urge sensation affected by nocturia or frequent urination, for example, whose sleep is interrupted by frequent urination intractable to conventional treatment, or for a public performer who must defer voiding until a proper receptacle can be used or drainage diverted to a collection bag voluntarily, where voiding is controlled manually by the user who rotates knobs mounted to a side of the mons pubis or mons veneris which advance and retract push/pull, or Bowden cables used to adjust valves of the type shown in FIGS. 5 and 6 or servovalves such as shown in FIG. 10A but without the fine adjustment device.

Accordingly, FIG. 30 shows not a prosthesis for a patient having undergone a pelvic evisceration as does FIG. 28, but rather a urinary assist, or voiding control device, which allows the patient with refractory nocturia, urinary incontinence, or frequent urination, or one who must participate in an activity which does not allow for interruption, such as public appearance or performance, to switch urine output from the innate urinary bladder to neobladder 145. Shown in FIG. 29, the device neobladder incorporates a strain gauge 152 that initiates emptying of the neobladder with the aid of turbine driver 150 to automatically empty the contents through urine outlet pipe 110 into collection bag 148.

Unshown, collection bag 148 has a capped emptying hose below to allow release into a bathroom receptacle. The capacity of collection bag 148 can hold far more urine than would be needed during a public speech or athletic event, for example. Following a surgical procedure on or involving the lower urinary tract, such a system is useful on a temporary basis for relieving the lower tract of constant exposure to urine by bypassing the healing tissue, thus expediting healing of the tract. For public performance, when switched to the automatic assist device, the wearer experiences no urge sensation as would be a distraction likely to prompt an interruption in performance, which for a concert violinist with an overactive bladder, for example, would significantly degrade the quality of performance.

To best expedite healing, postprocedural bypass can be continuous or intermittent and automatically coordinated with the automatic pipeline targeted release of a drug, during which diversion is not used, or during prosthesis disinfection or crystal dissolution, during which diversion is not used. When administering drugs, the controller switches off the bypass feature, thus gaining access to the native tissue. When administering system maintenance substances, the controller switches on the bypass feature, thus causing the substance or substances to flow through the synthetic device. Use thus is possible whether the urinary diversion system is implanted for the procedure or was already in place.

Such use is similar to the use of a carotid prostheses such as shown in FIGS. 25A and 25B following a carotid endarterectomy, for example, where bypass to expedite healing and expose or bypass the native tissue as required can likewise be continuous or intermittent. No less pertinent for expediting healing is the capability to directly pipeline target drugs such as anti-inflammatory, antimicrobial, and/or analgesic to the treatment site, thus precluding exposure to potentially harmful substances of tissue outside the urinary tract, thus avoiding adverse side effects and the risk of complications, while at the same time allowing the use of dose levels in the treatment of the lower urinary tract not restricted due to this consideration.

If explanted, leaving in place the body surface port, controller, druglines, and accessory channels of lower urinary tract and carotid prostheses by replacing the valves with basic ductus side-entry jackets allows continued automatic targeted treatment, if necessary, to the end of life even in a younger patient. Such extended use is made possible because the system controller can schedule the periodic release of substances to maintain the system itself, such as anticoagulants, thrombolytics, and crystal dissolvents on the same basis as drugs.

Except in cases of megaureter where servomotor-driven diversion chutes controlled by potentiometers in place of mechanical control knobs 153 and 153′ positioned around urine effluent line 110 (which in turn is connected to urine effluent hose 149 for drainage into urine collection bag 148) at the center of surface port 104 positioned to a side of the mons pubis or mons veneris, the diversion chutes in valves 143 and 143′ need not advance and retract more than 5 millimeters. This small excursion allows a simple in-line direct drive mechanical embodiment providing continuous adjustability, not bistable as would a solenoid, part number 32 in the drawing figures, to be produced without the expense of servomotors.

In the front schematic view of such a mechanically controllable embodiment shown in FIG. 30, push/pull cable rotating knob 153; controlling side-entry diversion valves of the kind shown in FIGS. 5 and 6 on the right-hand side of the patient and valve 153′ controlling that on the left rotate proximal twisted strip segments next to be described (behind control knobs 153 and 153′ and unseen in FIG. 30) at the proximal end of push/pull control cables 28 and 28′, also shown in FIG. 5 depicting the type valve used. So that one half turn of knobs 153 and 153′ clockwise fully extends push/pull cables 28 and 28′ respectively, while one half counterclockwise rotation fully retracts each, the turns ratio of the knobs to chute displacement is best one to two; that is, so that the halfway rotation of the knobs moves the diversion chutes from one end to the other, not just halfway.

Requiring to control movement over a distance no more than millimetric, the push/pull control cable extension and retraction mechanism inside port 147 in FIG. 30 providing control knobs positioned as would injection openings or button cells about urine effluent line 110 as shown in FIG. 26C can be made small enough to allow the one body surface port to include subdermal injection openings and button cells as well as control knobs. The twisted strip segment of each control which is separate between the sides consists of two turns covering an overall length one millimeter greater than the equivalent excursion sought of the diversion chutes and is distinct from the proximal ends of the push/pull cables fused to the rear of the ferrules.

In the control for each side, the forward or distal of the two turns of the twisted strip is contained inside, and the near (rear, proximal) turn outside its respective ferrule. The ferrule has a rectangular opening at the front end so that rotation of the twisted strip clockwise causes the outer turn to drive the ferrule, and therewith, the push/pull cable ending at the valve continuous with it forward over the distance equivalent to the entire displacement required of the diversion chutes. The outside turn drives the cables forward and the chutes deeper into the ureter, and the inside turn pulls the cable backward, withdrawing the chutes from the ureter.

Along with the clockwise or forward detent of the control knobs, contact of the rear end of the inside turn against the rear wall of the ferrules sets the limit to movement of cables 28 and 28′ in the forward or chute deployment direction. Along with the counterclockwise or backward detent of the control knobs, the limit of backward (chute withdrawal equivalent, retractive, proximal) chute movement is set by contact of the turn in front of the ferrule with the rear of the control knob.

Accordingly, the rear ends of the ferrules fused to and continuous with the solid cables, clockwise rotation of knobs 153 and 153′ causes the respective twisted strips to drive the ferrules and thus the cables and diversion chutes continuous with these forward, or distally, deeper into the lumen of the substrate ductus, while rotation of the knobs counterclockwise draws the ferrules proximally and therewith, the cables and the chutes retracted from the lumen of the substrate ductus. The small distance for the cables to move thus allows not only the avoidance of the cost for servovalves but the need for a lever mechanism likely to catch on clothing or cause an occasional scrape.

### Anastomosis Coupling or Flow Diverter

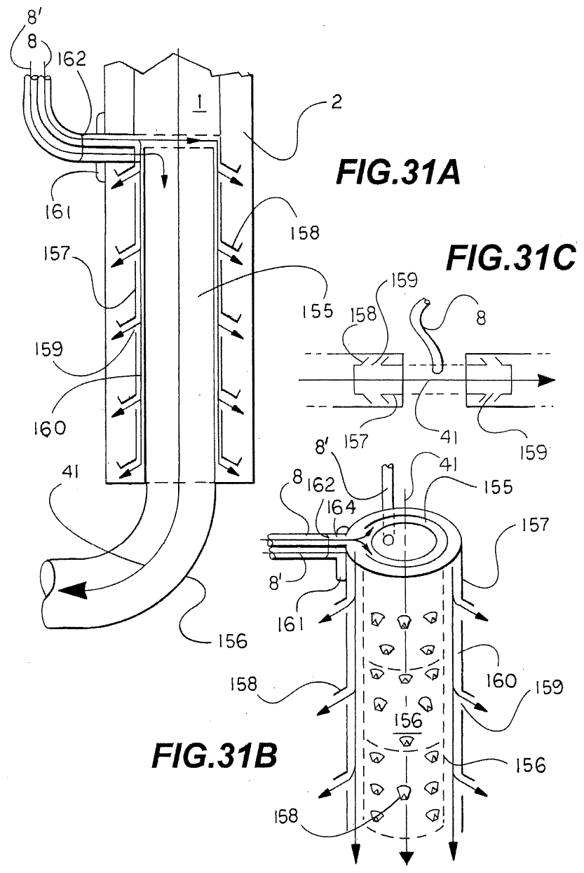

The functionality and functions of endoluminal anastomosis coupling or flow diverters in comparison with that of side-entry diversion jackets is addressed above in section 2. Background of the Invention, which briefly describes their structure. FIG. 31A shows a sectional view of an end inserted coupling used as a flow diverter inserted into the end of a native ductus of which 1 is the lumen and 2 the wall, while FIG. 31B shows a front perspectival view of an end-inserted coupling. FIG. 31C shows a bendable non-valve ductus flow diversion coupling which has been straightened for use as an inline end-to-end anastomosis junction coupling.

Summarizing, in FIGS. 31A and 31B, part number 8 is an accessory channel delivering drugs or other agents into the space between the inserter and the endothelium or urothelium, pursuant to which it makes a right angle turn down along the inserter, 8′ an accessory channel delivering drugs or other agents into the end-inserter lumen, which flow down the interior wall of the inserter lumen, 41 is a blood or urine stream or flow line, 155 designates the end-inserted endoluminal flow diverter in its entirety, 156 the inner and 157 the outer wall of double-walled, or double-tubed, end-inserted coupling or flow diverter 155.

Prongs 158 are die-cut and pressed or molded in a tough polymer such as polyethylene terephthalate outward at an angle serve both to anchor the coupling within the native lumen as well as to steer drugs or agents delivered through accessory channel 8′ and down the space separating the inner 156 from the outer wall 157 of end-inserted coupling 155, 159 are the spaces that serve as the drug outflow openings, or apertures created by the die or mold when prongs 158 are punch cut and pressed at an outward angle, 160 is the space separating inner wall 156 from outer wall 157 down through which drugs flowing down accessory channel 8.′

Where accessory channel 8′ is vertical, it is structurally and functionally synonymous with intertube space 160. Grommet 161 secures to stabilize, seal, and maintain leak-free the junction between the entry into the end-inserter of accessory channels 8 and 8′, and 162 represents the joint connecting accessory channels 8 and 8′ to end-inserter 155 at accessory channels/double tubular connection extension 164. FIG. 31C shows an end-inserter coupling used to connect the stumps of the same ductus or different ductus, the difference between use as a bridge or as a means of forming an anastomosis just the difference in distance separating the transected ends of the stumps.

Accordingly, two accessory channels are provided, one, part number 8, delivers drugs into the intertube space, or the passageway between inner tube 156 and outer tube 157 so that under the slight to moderate pressure of the implanted drug reservoir outlet pump or drip stopcock, these flow around and down intertube space 160 to emerge through apertures 159, one each created by the mold or die punching outward of a pointed retention tine (barb, prong) 158.

Made of a suitable material such as sterile polyethylene terephthalate free of any irritant such as residual plasticizer or surfactant on the surfaces, the prongs provoke the development of a protective fibrous capsule. Drugs such as anti-inflammatoires delivered through accessory channel 8′ flow over the urothelium, tunica mucosa, or endothelium of the substrate ductus for absorption and uptake, not just at apertures 159, while accessory channel 8′ delivers an anticoagulant, for example, into the coupling lumen within the native lumen in vessels and a crystal solvent, for example, in the ureters.

Double, or dual, lumen tubular extension 164, which protrudes radially outward from the adventitia no more than is needed to securely friction fit druglines and accessory channels 8 and 8′, opens adaxially into intertube space 160 separating inner tube 156 from outer tube 157. To avoid the need for a second puncture wound through luminal wall 2, the druglines connected to tubular extension 164 arrive through double lumen catheters, or the lines flowing into accessory channels 8 and 8′ can be separate and run in parallel from the drug sources, normally, small flat drug reservoirs implanted in the pectoral region.

Grommet 161 then encloses the single perforation. To prevent incising the tunica mucosa, urothelium, or endothelium when the endoluminal anastomosis coupling or diverter is inserted, double lumen tubular extension 164 has a smoothly burnished end 162, and along with downstream inclined retention tines (barbs, prongs) 158, is blanketed over or with a coating of a readily absorbable substance such as a sugar or chilled butter, which can be quickly dispersed by passing water or enzymes respectively through apertures 159. Flowline 41 indicates the direction of flow through inner tube 156.

Slightly protrusive, extension 164 is easily spotted and palpated at the surface of the adventitia as a slight eminence, showing the operator the point to remove a plug of tissue no larger than is needed to allow double lumen extension 164 to protrude out through the side of the adventitia. Grommet 161, incorporating or coated with an anti-inflammatory and antimicrobial polymer, is then placed in surrounding relation to double lumen extension 164 against the adventitia to completely enclose this junction and prevent microbial intrusion.

When inserted into the cut end of the native ductus, the tube within a tube flow diverter or adventitia coupling as shown does not allow switching flow between the diversion path and that normal. For this reason, it is functionally distinct from a side-entry diversion jacket, which is more versatile in flow control. By the same token, a straight, or nondiverting, embodiment of the device has applications of which the periductal jacket is incapable, such as transluminal placement as a stent or anastomosis coupling. Both allow the diversion of flow through a shunt or bypass, but only the diversion jacket allows the outflow to be divided in any proportion between the native ductus and the shunt or bypass.

### Long-to-Short Bloodline-Switch Device

FIGS. 32A thru 32F show the sequence of steps for switching from a longer to a shorter bloodline with no interruption in blood flow using a bloodline switch. FIG. 32G provides a detailed view of a bloodline-switch of which one at either end of the bloodlines as depicted in FIG. 32A is used to transfer blood flow from the longer of two lines. For visual clarity, only the venous line, corresponding to that lower in FIGS. 22A and 22B, is shown. Here the anastomosis drip drug delivery line is numbered 154 rather than 8 or 8′ to make it clear that this need not represent the terminus of the accessory channels labeled 8 or 8′; but rather valve-jackets amenable to accommodating multiple accessory channels, another accessory channel.

The reverse use of line switching from the short bloodline 174 to the long bloodline 170 is equally valuable in:
1. Accommodating growth when an unforeseeable circumstance necessitates a degree of expansion of the prosthesis beyond that predictable at the time of implantation when the patient was an infant. Insertion of a longer line 140, however, can usually be accomplished endoscopically, sparing the patient a thoracotomy, for example. The longer line can also be chosen with a larger caliver, or diameter.
2. A midprocedural mishap justifies aborting the procedure requiring its reversal.
3. A midprocedural mishap that makes an increase bloodline length necessary or beneficial.

That the same concept can be applied to any requirement for the ability to alter line length without an interruption in flow is considered obvious.

In FIGS. 32A thru 32D, the recipient organ is labeled 172 and the donor 173. Depending upon the distance between recipient bloodline-switch 168 and donor bloodline-switch 169, inline pump 167, of the type used in ventricular assist devices, inserted within long bloodline 170 is used to assist in propelling the blood past the distance that pressurization through the use of inotropics and/or mechanical assistance of the impaired recipient heart would allow. Because unlike FIGS. 22A and 22G which show both the arterial and venous lines, for schematic simplification, FIGS. 32A thru 32F show only that venous, inline pump 167 is situated more toward the donor side; if shown, an arterial pump would be shown more toward the recipient side.

Upon inception of the procedure, the distance between donor or graft organ 173 and diseased, malformed, or severely injured recipient organ 172 is spanned by long bloodline 170. When applied to vessels such as the coronary and carotid arteries which when handled might vasospasm or induce a dysrhythmia, for example, the bloodline-switch key shown in FIG. 34A allows switching from donor venous long line 170 to donor venous short line 174 in the step depicted in moving from FIG. 32A to FIG. 32B through a continuous transfer of blood flow from the one line to the other without a transient change in blood pressure.

In the retum, or venous, flow depicted in FIGS. 32A thru 32F, the vein of the donor is 194 and 197 the synthetic bloodline shown as integral with the donor bloodline switch to which is connected valve indicated by its flow diversion chute 18, the corresponding vein of the recipient is 194′ and the bloodline associated with its valve is 197′. In FIGS. 32D thru 32F, 46′ is the anastomosis joining donor and recipient veins. The reverse arterial line has been omitted as identical but flowing in the opposite direction as well as to allow for visual clarity.

A compound vascular bypass transplant requiring the connection of each vessel of the donor to the corresponding vessel of the recipient, the long and short lines depicted in FIGS. 32A thru 32F are both bloodlines, these representative of each pairing of the supply and drainage bloodlines between the donor and the recipient. In these figures, part number 170 represents the donor venous long bloodline, and 174 the donor venous short bloodline. While the heart requires numerous such connections, these will usually be merged for visual clarity through the interpositioning of confluence conduits as shown in FIG. 35B, imparting ability to segregably medicate the vessels combined thus through a common drugline, part number 8 in FIG. 35B leading to a common accessory channel.

In comparison, the bypass transplantation of any other solid organ calls for far fewer such connections. Unless having been implanted with a prosthetic disorder response system, the donor would always be without druglines, or sidelines, and if present, these would play no part in the organ transfer and not require long to short line switches. Since a transplant denotes the antecedent failure of a major organ, such as an automatic response system may have been implanted in the recipient to deal with that preliminary condition prior to the transplant. For an organ transplant, which signifies that the organ had been failing, the recipient might have been implanted with.

If not, such a system is enlisted to administer the transplant and should remain implanted to monitor the state of the graft organ and release immunosuppressive and other supportive medication as necessary. End stage organ failure will mean that as interdependent with the organ to be replaced, the other organs will likewise have become impaired, and this means that the disorder response system will not be a simple but rather a microprocessor-administered multimorbid automatic response system. To avoid the need for reentry and the delay to allow an interval for recovery, the automatic response system is best implanted at the start of the transplant.

Due to the number of lines required for a heart transplant, to have one team working on implanting the disorder response system and another on the transplant simultaneously is needlessly chaotic and conducive to human errors. Instead, the disorder response system should be accomplished and the wound not sutured but left open with a protective dressing while the patient is given time to heal. In an adult, the smaller lines-those to be implanted—are made of clear transparent polypropylene tubing, for example, free of any potentially imitating surfactant or plasticizer residue.

In a child, where the smaller line must expand in step with growth the short bloodline 174 is made using the expansion structure described above in section 2b(2)(f), entitled Expandable Structure to Accommodate Growth and Self-adjust in Size in Vascular Segment Prostheses, Lines, and Confluence Conduits Placed in Children. To assure expansion in step with growth, when short line 174 is to be implanted in a neonate, infant, or small child, it is made using the expansion structure laid out above in sections 2a., entitled, Background of the Invention, 2b(2)(f), as just stated and below in section 6, entitled Description of the Preferred Embodiments of the Invention, which provides an end-tensile strength (rupture/bursting) force sufficient to withstand a direct impact.

This is distinct from a shorter confluence chamber that will be implanted and must expand to accommodate growth as illustrated in FIG. 15D and addressed above in section 2b(1)(b)(b15), entitled Switchable Lines and Confluence Conduits: Long when Intercorporeal, to Short when Intracorporeal, Both Affording Connection of One or More Bloodlines to the Same or a Different Number of Bloodlines with Self-leveling Apportionment of Outflow, and shown schematically in FIG. 35B. That sterility and sterilizability are basic is superfluous. Ordinarily, not just the tubing but all parts of the line switch assembly are made of residue-free clear transparent polypropylene or a similar material.

### Interposition of a Flexible Joint in a Short Line to be Implanted

For placement as the shorter of two lines to be implanted as part of a transplant, if short bloodline 174 will have to clear another implant or otherwise have to negotiate a curve sharper than would be necessary to clear the bent waist (of which an expansion structure will remain capable throughout growth), the necessary degree of flexibility is obtained by interposing a flexible joint no larger than would be necessary in the adult. Another point of flexion is that of the popliteal vessels. Made of polypropylene or polyetheretherketone convoluted tubing, the flex joint is surface treated as are the expansion segments, to repel adhesions, biofilm, adsorption, and breakdown. Not immune to such degradation in the body, measures are taken to isolate these materials.

That short line 174 can be of the expansion structure form in an organ transplant with a flexion joint placed with the aid of line switches is not to suggest that the same kind of expansion structured line with a flex joint cannot also be implanted apart from an organ transplant with the aid of inline coupling jackets. The structure of a flex joint is the same in either case. Nonexpanding, hence, necessarily of adult size for placement in an infant, the interposed flex joint is segment is about ¾ inch, or 19 millimeters long and 4-5 millimeters in internal diameter and joined to the much narrower expansion structured segments with the aid of adapters.

Connection of the short, convoluted flex joint segment to the inner ends of the smaller-1-2 millimeter internal diameter-expansion structure to either side is with the aid of adapters. The outer ends of the adapters must match the small inner ends of the expansion structure in diameter, while their inner ends must match the larger ends of the adult sized flex joint. That is, as the child grows, the outer ends of the adapter must expand with the expanding ends of the expansion structure to which they are fused so that in the adult, the train of parts, expansion structure, adapter, flex joint, adapter, expansion structure will become cylindrical from one end to the other.

Such adapters can be made by fusing short segments of an implantable readily stretched artificial rubber-like material of the smaller size to the larger proximal ends of the divided expandable line. To reduce the degree of inherent stretchability, the adapter segments can be molded in the shape required with the end shape attained through elasticity. Whether still unexpanded or in the adult, the interposition of a convoluted joint at a tight bend will result in turbulent flow through it; however, the flow should be able to reinstate itself to laminar before reentry into the native vessel to which it is connected, and since the turbulence was confined to the synthetic segment, no harm should result.

Provided the materials chosen are inherently or can be modified for weldability, the joints and tapered adapters are plastic welded together or are bonded together by solvent bonding. As the child grows, the adapter elements expand with the ends of the expansion structure to which they are fused until these smaller ends match the adult segment in size. Materials of the joints not immune to degradation over time when implanted, with an end-structure meant to endure to the end of life, means must be provided for isolating these from the surrounding milieu.

Subject to degradation over time, polypropylene or polyetheretherketone are parylene coated (see, for example, Hao, D., Lin, J., Liu, R., Pivetti, C., Yamashiro, K., and 9 others 2023. ““A Bio-instructive Parylene-based Conformal Coating Suppresses Thrombosis and Intimal Hyperplasia of Implantable Vascular Devices,” Online, Bioactive Materials (Beijing, China) 28:467-479; Stemschuss, G., Ostergard, D. R., and Patel, H. 2012. “Post-implantation Alterations of Polypropylene [finely woven mesh, not solid material] in the Human,” Journal of Urology (Philadelphia, Pennsylvania) 188(1): 27-32).

Current parylenes, vapor deposited coatings not available as sheet stock or extruded in tubular form, have been developed which can stretch by about 170 percent, which for the present purpose that calls for a fivefold stretchability, is inadequate (see, for example, Xu, H., Zhang, M., Chen, L., Zhang, P., Jin, Y., and Wang, W. 2023. “Silicon-based Stretchable Structure via Paylene Krigami Interconnection,” [Institute of Electrical and Electronics Engineers] Journal of Microelectromechanical Systems 32(1): 82-90; Takei, A., Tsukamoto, S., Komazaki, Y., Kusaka, Y., Kuribara, K., and Yoshida, M. 2020. “Stretchable and Durable Parylene/PDOT-PSS [3,4-ethylenedioxythiophene polystyrene sulfonate]/Parylene Multi-layer Induced by Plastic Deformation for Stretchable Device Using Functionalized PDMS [polydimethylsiloxane],” Online, American Institute of Physics Advances 10:025205).

This consists of wrapping the joint from just short of the inner ends of expansion structure within a parylene-coated thin plasticized polypropylene or polyetheretherketone sheet to create an artificial nonstretching bursa. Slightly larger in diameter than the adult-sized convoluted flexion joint, the parylene coated bursa has plain washer-configured ends bonded to the inner ends of the baby-sized expansion structure and the cylindrical portion enveloping the flexion joint. These bonds must be produced under stringent clean room conditions (see Specialty Coating Systems Corp. 2023. “Common Parylene Problems: Issues that Complicate Parylene Coating,” Online at https://www.paryleneconformalcoating.com/conformal-coating-education-center/parylene-disadvantages/#failure, Johnstown, Pennsylvania: Diamond-MT Division, Kisco Corporation). The leakproof ‘bursa’ can be filled with pure distilled water, ethanol, or propanol, for example, or can be left empty.

To prevent disengagement of the lines from the sockets or receptacles 171 in FIGS. 32A, 32G, 33A, and 33B in donor bloodline-switch in 169 or recipient bloodline-switch 168 are provided with roughly textured termini 171 which demand the application of more than moderate force either to insert (engage) or extract (disengage). Bloodline-switch (or bloodline-switch) flow stoppers 175 at venous inlet long and short bloodline-switch 169 (on the left-hand side of FIGS. 32A thru 32C) and 176 at the venous outlet bloodline-switch are initially switched downward to close off short bloodline 174 at both ends.

As shown in FIG. 32B, once donor and recipient are brought alongside one another allowing use of short bloodline 174, long line 170 is flexed allowing the outlet end of venous short line 174 to be inserted into short line outlet socket 171. Shown in FIG. 32B, bloodline-switch flow stoppers 175 at the venous inlet and 176 at the exit, or outlet, line switch are now switched upward to close off long line 170 and switch blood flow through short line 174, long bloodline 170 then removed.

Exact synchrony in the switching at both ends of the bloodlines to minimize if not eliminate any greater than instantaneous transient in blood pressure which if associated with blood flow to the coronaries or carotids could provoke a vasospasm and/or dysrhythmia, even a sudden arrest, is achieved through use of a microminiature socket wrench key-device such as that shown in FIG. 32H which spans both of the small sockets between line stopper switch square or hex axle-turning heads 177 on the left or donor venous end, and 177′ on the right or recipient venous end to allow both to be wrenched simultaneously.

Because:
1. At the moment of switching from the long to the short line, line switch blood confluence chamber (hollow, well) 178 seen in FIG. 32G is already full,
2. Switching can be accomplished instantaneously, and
3. Pump 167 can be used to assist in sustaining flow with no more than an instantaneous dip in blood pressure.

As a result, blood flow throughout transplantation is substantially uninterrupted. Referring now to FIG. 32C, blood flow to the recipient never having been interrupted, irreparably diseased, malformed, or injured native organ 172 is removed and long bloodline 170 having since been removed, short bloodline 174 is now bent to allow the donor graft organ and associated venous outlet components to be suitably rotated into position for orthotopic placement above recipient line switch 168, shown in FIG. 32D.

Graft organ and recipient veins are now anastomosed, with donor accessory channel in position above venous anastomosis 46′ to release immunosuppressive, anti-inflammatory, or antimicrobial drugs automatically from the sending implanted drug reservoirs as commanded by the implanted microcontroller or microprocessor in response to sensor feedback indicating the presence of a one or more biomarkers of an adverse reaction.

Vascular servovalves, diversion valves, servochokes, and basic ductus side-entry jackets accessible through plural accessory channels, the labeling of this accessory channel positioned above to automatically wet anastomosis 46′ (corresponding anastomosis 46 in the arterial line, not shown for visual clarity, is implied) with medication according to the system prescription-program in response to adverse reaction biomarkers signaled by the implant sensors 154 is meant to indicate that it is not necessarily the terminus of accessory channel 8. The function of switching of blood flow between long and short bloodlines should not be confused with switching the flow of blood from undiverted to diverted by extending vascular valve or servovalve diversion chutes.

In combination, these two switching points or levels can alter the relative proportion of blood diverted and passed through either long bloodline 170 and short bloodline 174. In FIG. 32E, the vascular servovalve diversion chutes as seen in FIG. 10A, for example, have been fully retracted allowing blood to flow without diversion through the donor and recipient veins anastomosed at 46′.

The fact that the implanted microcontroller (in isolated monomorbid disease) or microprocessor (as the implanted master controller of a hierarchical control system in comorbid disease) can automatically retract diversion chutes 18 to switch blood flow between partial or full through-flow as shown in FIG. 32E and partial or full anastomotic bypass flow as shown in FIG. 32F has significant implications. For example, notwithstanding the direct pipeline targeting of remediating agents to the site through accessory channels, were the anastomosis or the segment containing it to become stenosed, inflamed, infected, or vasculopathic, flow would be automatically switched from through-flow to bypass flow around the affected segment.

By the same token, notwithstanding the direct pipeline targeting to the synthetic bypass of an anticoagulant, anti-inflammatory, crystal solvent, or antimicrobial, were the bypass to become adversely affected, the passage of blood would be switched to through-flow. Moreover, once the system responded to the exigency, flow could be automatically switched back to that at the outset. As shown in FIG. 32F, undiverted flow allows the potentially obtrusive bloodline-switches to be replaced with a short and little protrusive valve-to-valve bloodline, or mainline 9.

FIG. 32G provides a more detailed view of the venous inlet line-switch seen as part number 169 in FIGS. 32A thru 32D. In FIG. 32G, the line-switch is labeled 169, the upper longer mainline usually identified as part number 9, but in this specific context, line 170, the shorter lower mainline usually identified as part number 9 shown as 174.

FIG. 33A provides a detailed view of the bloodline-switch flapper-like dual or back-to-back stopper 175 in its initially downward position as shown in FIG. 32A, closing off lower short line 174, and in so doing, relegating venous blood return from the donor to the recipient as shown in FIG. 15A through upper long line 170 before donor and recipient can be positioned alongside one another. FIG. 33B then, shows venous stopper 175 after having been switched to close off short line 174. For visual clarity, the corresponding arterial and receiving venous switches at the right-hand, or recipient, end have been omitted. It should be understood, however, that in FIGS. 33A and 33B, the recipient arterial and receiving venous bloodline stoppers at the right-hand end in FIGS. 32A thru 32C work simultaneously as the antipodal complement to those shown at the left-hand donor end.

As shown in FIGS. 32A and 32B, once the donor and recipient can be positioned alongside one another, the return of venous blood from the donor to the recipient is switched from the long upper line 140 to the shorter lower line 147, two-sided Janus-configured stopper 175 simultaneously rotated from the downward to the upward position to close off longer upper line 170. That the relative positions of the long line 140 and short line 147 as to which is the upper and which the lower could be reversed or as to the relative positions of the donor and recipient as to which is positioned on the right and which to the left is considered obvious.

FIGS. 33A and 33B provide a more detailed view of a two-sided bloodline-switching two-faced ir Janus-formed stopper 175, made of a rubbery polymer of which either side is similar in presenting a centrally shallow domed, or convex area with a flat sealing surround, but tiny compared to a toilet tank flapper-type stopper.

To afford the operator a clear and unobstructed view of blood flow through the line switches and lines and thus allow ultrasound or endoscopic assisted visual, or in an open field, direct visual confirmation that switching is fully rotating both two-sided stoppers 175, thus completely opening or sealing off either long line 140 at the same instant that it is sealing off or opening short line 147, line switches such as 168 and 169 are injection molded in a clear transparent plasticizer and surfactant residue-free polymer such as polypropylene.

Referring now to FIG. 32A, stoppers where blood confluence chamber 178 empties into either long or short line opening of the bloodlines, or mainlines, serve to close off lower line 174, then in FIG. 32B, switching flow from upper long line 170 to lower short line 174. That is, stoppers 175 at either ends of lines 170 and 174 when rotated between the downward direction in FIG. 32A to the upward in FIG. 32B simultaneously close off upper long line 170 at both ends while opening lower short 174 at both ends. To prevent recurrent rather than straight-through flow at a line outlet, not just the sending line but also the receiving line require line switches with stoppers 175 on the donor left and 176 on the recipient right, stopper switches 169 on the venous donor left and 168 on the recipient right.

### Bloodline-Switch Key

The use of a bloodline-switch key with bloodline-switches such as 168 on the recipient right-hand side and 169 on the donor left-hand side of FIGS. 32A thru 32C is intended primarily for vessels which to minimize any transient in blood pressure as might provoke a vasospasm of dysrhythmia, for example, should not be manipulated, and more particularly clamped. As for arteries not this sensitive, provided the operator can manually rotate the stopper rotating bolt-like heads in quick succession, the need for a switch key is eliminated.

FIG. 34A provides a detailed overhead view of a dual socket wrench key-device for simultaneously rotating the polygonal, usually hexagonal, stopper bolt-like rotating heads 177 on the left or donor venous end, and 177′ on the right or recipient venous end, that of the donor to the left clockwise and that or the recipient to the right counterclockwise, most often to switch blood flow from the inlet and outlet of long line 170 to the inlet and outlet of short line 174. The manually used key comprises sockets at either end complementary in distance apart and polygonal axle head conformation to the distance separating and the shape as the negative (receiving, female) complement to stopper rotating bolt-like heads 177 on the donor left and the recipient right in FIG. 32B. In FIG. 34A, bolt head-like polygonal stopper rotators are 183 on the left and 183′ on the right.

Detent mechanism 188 shown next in FIG. 34B intervening, centered control knob 179 rotates control knob drive sprocket 180, which together with its complementary antipodal or take-up sprocket 182′ rotate shaft 189 connecting take-up sprocket 182′ to stopper bolt head like rotator 175 while at the same time lengthened to also serve as a spacer between the two, thus driving simple ladder-configured drive chain 181 to rotate bolt head configured stopper rotator 183′ clockwise. Mounted on the same shaft, drive sprocket 180 also moves spur gear 190 meshed with its counterpart 191 to move drive chain 192 as is essential to rotate stoppers 175 and 176 shown at either end of FIG. 32B, for example, in opposite directions.

Accordingly, center left sprocket 193, mounted on the same shaft as left spur gear 191, drive chain 192, left end sprocket 182, hence, left end stopper bolt head like rotator 175 (shown in FIGS. 32A thru 32D, and FIGS. 33A and 33B) and the corresponding elements on the right-hand side rotate stoppers 175 simultaneously in opposite directions. As illustrated in the step taken between FIGS. 32D and 32E, bloodline-switches as well as small wrench or tire iron dual socket wrench-configured key devices are used only during the time that donor and recipient remain connected. After that, both are recovered and can be flushed through with a heparin solution and sterilized by autoclave, ethylene oxide (oxirane) gas, or by placement in an ultraviolet light sterilization chamber for reuse.

In FIGS. 32B and 34B, bolt-like configured rotating bistable stopper 175 turning heads 177 and 177′; used to switch between long line 170 and short line 174, must be rotated simultaneously or the flow of blood through the lines will be disrupted. The actuator positioned to rotate bistable stopper 175 unseen as on the back side of stopper axle 177 is a miniature rotary solenoid. Capable of inducing a vasospasm or a desynchronization, for example, this eventuality is to be stringently avoided. Exact synchronization in the rotation of long-to-short line switch stoppers 175 and 176 respectively, as shown in FIG. 32B is by means of the key device shown in FIG. 34A which rotates stoppers 175 and 176 simultaneously. Referring to FIGS. 32B and 34A, small socket head 183 at the left end of the key tool, receives left stopper turning nut 177 in the bloodline switch, while socket head 183′ on the right engages stopper 177′ on the right, so that rotating key control knob 179 rotates these simultaneously in opposite directions.

Referring now to FIG. 32B, using the key device shown in FIG. 34A to rotate stopper 175, at the left—the donor venous return switch entry end-counterclockwise, while at the same time rotating stopper 176 at the right, or donor venous receipt entry end clockwise, switches the flow of blood from the longer donor venous bloodline 170 to the shorter line 174. Switching thus allows increasing the distance between the donor and the recipient until the donor organ is harvested and brought to the recipient, where it is preferred if not necessary to reduce the length of the bloodlines. Reverse switching to allow blood to flow from one patient to another over a distance is possible. Longer distances are likely to necessitate the use of an inline, or endovascular, pump, shown as part number 167 in FIGS. 32A and 32B. e

### Bloodline-Switch Key Detent Mechanism

FIG. 34B provides a detailed front view of a detent mechanism 188 suitable for providing sensibly indexed rotation of the bloodline-switch socket wrench key-device handle 179 to simultaneously rotate both bolt head-like switch stopper rotators 177 on the left or donor venous end, and 177′ on the right or recipient venous end, thus rotating stopper 175 on the donor left and 176 on the recipient right, as well as detent mechanism 188 in FIGS. 32A thru 32C, 34A, and 34B. The actuator positioned to rotate bistable stopper 175 unseen as on the back side of stopper axle 177 is a miniature rotary solenoid. That parts of the strip-spring alignment mounting to include strip-spring radial alignment posts 185, cross tie pins 186, and strip-spring 184 itself can be produced either through insert molding or, except for stainless spring steel strip-spring 184, formed as a relief integral in the material of the mold is familiar to those skilled in the molding arts.

In FIGS. 34A and 34B, detent 188 is positioned just behind, here, recessed within the front surface, of switch socket wrench key-device handle 179, the key-device used to simultaneously rotate bolt-like stopper rotating heads 177 on the left and 177′ on the right from the upper longer donor retum-to-recipient venous line 170 to the shorter lower donor venous line 174 or, should a long period of growth of a midprocedural mishap necessitate, the reverse. The detent matches the equilateral polygonal, that is, square, pentagonal, or hexagonal rotating tiny bolt-like rotating heads 177 on the left or donor venous end, and 177′ on the right or recipient venous end on the outer surface of line-switches 169 at the donor end to the left and 168 at the recipient end to the right.

Detent polygonal cap-head shaped frame 177 exactly matches the polygonal bolt-like heads on the outer surface of the sending and receiving bloodline-switches at either end of the bloodlines and encloses stainless spring steel spring, strip-spring 184, enclosing it so that the bottom edge or margin of detent cap-head 177 moves just above the outer surface of the bloodline-switch. As each reentrant of stopper 175 axle rotating polygonal cap-head 177 passes, the apical tip terminus of strip-spring 184 snaps from bent to substantially straight so that the tip comes to lodge in the angle of polygonal cap-head 177. This abrupt engagement generates an audible click accompanied by a sudden resistance to continued rotation and thus serves as a detent.

A typical confluence conduit is shown in FIG. 35A and a switchable embodiment in FIG. 35B. To assure that the extension arms leading to the valves at either end never become detached, each type confluence conduit with respect to the number of such extensions at either end must be unitary, the extension arms never connected to the main body by means such as plastic welding or an adhesive. When different, the number of inlet and outlet arms will usually respond to anomalous supernumerary or accessory branches. Such deviations from the norm are commonly encountered with the blood supply and venous outflow of kidneys, which matter is addressed shortly below in the section.

### Inline Coupling Jackets in the Context of Bridging, or Tie-Lines

Bridging, or tie-lines are prosthetic segments interposed along native ductus to include vessels and ureters where a segment along a ductus, ordinarily a native vessel or a ureter, had to be excised due to disease or malformity, and confluence conduits are used to segregate a number of vessels for treatment distinct from that applied to others. These connectors couple native to synthetic ductus or conduits so that the coupling valve-jacket at the downstream or exit end is identical to that at the entry end but because the connection at the entry end is from native to synthetic while that at the exit end is from synthetic to native, each connector is positioned in the reverse direction relative to the other.

For simplicity, in the description of the automatic action that replaces a defective or diseased segment of a vessel, usually along the aorta, to follow, components identical in the entry and exit jackets and depicted as such in the accompanying drawings are identified in the singular, while parts shown in the drawing figures are assigned the same part number, the two distinguished through the addition of an apostrophe.

Inline coupling jackets are native-to-synthetic and synthetic-to-native ductus connectors of slimline conformation and compliant casing to allow placement without sideways protrusion into neighboring structures. This shape factor is exchanged for the ability of side-entry devices to incorporate a switch that allows flow to be directed into either of two passageways, which capability is indispensable for compound vascular bypass, or sudden switched-organ transplantation-type solid organ transplantation, for example. In the drawing figures, part number 198 denotes a prosthetic tie-line regardless of type, whereas part number 196 denotes a bloodline, and part number 230 a confluence conduit whether tubular or anatomical.

FIGS. 35A, and 35B show that multiple inputs or outlets to a confluence conduit require valves, not inline coupling jackets which are used to replace and secure a segment of a ductus with a prosthetic segment as a bridge or tie-line at either end. That is, while side-entry devices such as vascular valves connect one or more native vessels to a confluence conduit, inline coupling jackets connect only one tie-line or prosthesis at either end for insertion to replace a native vessel.

As addressed above in section 2b(2)(c)(c3) entitled Types of Inline Coupling Jacket-secured Bridging, or Tie-lines, inline coupling jackets are best addressed in the context of connectors for bridging, or tie-lines and tie-line type prostheses such as that shown in FIG. 36F. In FIGS. 36A and 36E, tubular or anatomically shaped synthetic tie-line 198 and in FIG. 36F, anatomically shaped synthetic tie-line 196 span the distance between the inlet side of the entry jacket 195 outlet half 200 to the inlet side of the inlet half 200′ of the exit jacket 195′.

In FIG. 36F, the prosthetic aortic neoarch is made with the expansion structure delineated above in section 2b(2)(f), entitled Expandable Structure to Accommodate Growth and Self-adjust in Size in Vascular Segment Prostheses, Lines, and Confluence Conduits Placed in Children. In FIGS. 36A and 36F, not structured thus are jacket halves 199 and 199′ which comply with growth internally by diametrical expansion into the yielding breakdown counteractant coated viscoelastic polyurethane foam. Part numbers consistent throughout and part number 230 denoting a confluence conduit, those in FIGS. 36A thru 36F represent the same components as defined in FIGS. 35A and 35B specified above.

FIGS. 36A, 36F, and 37F provide views of representative bridging line-based prostheses to replace a severely malformed segment along a vessel, that shown in FIG. 36F an expandable neoaortic arch with supraaortic branches where that native had been congenitally affected with an interrupted aortic arch, for example, to the extent that the operator would conventionally be compelled to perform a patch aortoplasty despite knowing that the limited expandability of such a repair would soon be outstripped by the rapid growth of the neonate who would require its replacement in the near term, likely before the small patient would have attained the robustness to withstand another critical operation.

To surmount this predicament, a more compliant and durable prosthesis, to include branching as necessary, is made available for any segment in any length along the aorta or any other native ductus large enough in caliber to warrant treatment thus. A prosthesis to repair an abdominal aortic aneurysm could incorporate a branch to accommodate the celiac axis, for example. The combination of inline coupling jackets to emplace and connect the tie or bridging line without clamping or much if any bleeding and the expandable rupture-resistant structure of these prostheses as represented in FIGS. 36A and 36F, for example, with migration-counteracting through-and-through stitching eliminates much trauma, considerably simplifies the operation, and yields a more durable result that should grow and last indefinitely.

For the neonate with a severe vascular defect, the prosthesis critically improves upon a stitched Dacron® (DuPont deNemours, Inc., Wilmington, Delaware) reinforced patch aortoplasty and any alternative available for the purpose. To the extent possible, such prostheses are standardized, and other than trimming back an excessively long branch, not modified by the operator. Instead, the prosthesis replaces not just the immediate defect, but inherently providing ‘extension for prevention,’ is placed over the affected segment to replace it in toto. This not only imparts a reduction in procedural complexity that allows general surgeons to undertake a repair that otherwise requires a highly skilled specialist at a large medical center, but precludes future complications.

For example, an aneurysm resulting from an extended weakening of an arterial wall not detected by imaging that would later fail and present another aneurysm would have been averted before such a development had the opportunity to eventuate. If not weakened at the time of the procedure, any postprocedural infection viral, bacterial, or mycotic could lead to another aneurysm without having presented a readily apparent cause such as a connective tissue disease. Accordingly, the replacement of some tissue that might have been preserved is more than compensated for by the relative simplicity of the procedure and prevention of adverse sequelae.

Shown in FIG. 36F is an aortic neoarch for a pediatric patient made with a considerably expandable and strong fabric devised to accommodate growth and described above in section 2b(2)(f). Expandable Structure to Accommodate Growth and Self-adjust in Size in Vascular Segment Prostheses, Lines, and Confluence Conduits Placed in Children; as well as in this section. In FIG. 36F, the supraaortic branch of the prosthesis for connection to the brachiocephalic, or innominate, artery to the left is labelled 201, that to connect the left common carotid 202, and that to connect the left subclavian to the right 203. The inline coupling jackets respective of each are generically numbers 195 for the single inlet jacket and 195′ for each of the outlet jackets, while the jackets securing the supraaortic branches are specifically numbered 204, 205, and 206.

Generically the substrate native branches would have been labelled 194, each of the inline coupling jackets as a whole labelled 195, and the halves thereof 199 and 200. Similarly, the inline coupling jackets to connect to these branch vessels are generically labelled 195 with the halves labelled 199 and 200, although for the general representation intended of FIG. 36E, distinctly labelled branches of the prosthesis are considered sufficient to distinguish the substrate vessel of each. The prosthesis branches are made long enough to replace a defect of the arch such as an aneurysm that extends up into the branch, any excess length of these easily determined by placing the prosthesis down against the prospective site for implantation and snipping the excess length with a scissors.

FIGS. 36A and 36E show a basic bridging, or tie-line, suitable for replacing a segment along a ductus such as a vessel or a ureter which a congenital or acquired structural defect made it necessary to excise. Along the vascular tree, that the bridge or tie-line spans the distance between and reconnects the cut ends of the substrate vessel without requiring the stopping of blood flow by means of clamping can be significant for averting complications.

As explained below in describing FIG. 37A, replacement of the native by a prosthetic segment with little if any blood loss is possible because the entry and exit inline coupling jackets rotate the native segment out and the prosthesis into its place in immediate succession so that the inflow of upstream or retrograde blood, obstructed from sideways leakage, is continuously transferred from the native and into the prosthetic segment. FIG. 36G shows a tie-line mounted vascular valve which can be used to provide an alternative mounting when no native vessel is present to serve as the substrate.

The procedure is always preceded by testing the transection mechanism in either jacket shown in FIGS. 36C and 37C as well as by ascertaining the degree of calcification if any of the substrate vessel. At or near to the brain or the heart where the calcium level indicates plaque of hydroxyapatite hardness or there is any other reason to suggest a ‘bailout’ procedure might become necessary, the patient is extracorporeal membrane oxygenator cannulized by a perfusionist enlisted before commencing and a conventional surgical repair performed. Well removed from the brain or the heart where a brief cessation in blood flow would pose no risk of injury, conventional clamping is used.

The implantation procedure already described in some detail above in the section entitled Background of the Invention, once the pin jacks have been inserted all the way into the pin jack sockets of the outlet half of the entry jacket and the inlet half of the exit jacket, the operator initiates the automatic completion of the procedure- and in so doing, eliminating the opportunity for human error—by pressing a switch on the system hand control.

This clears the trips that had prevented the razor from prematurely incising the native ductus and initiates the cutting action of the razor. In FIG. 36A, that the replacement prosthetic vessel segment 198 at the center incorporates the expandable alternating circumferential flats 233 and vaults 234 structure indicates that the prosthesis is for placement in a small pediatric patient to expand with growth and thus eliminate the need for revision over a period of years if not for life.

Whereas inline coupling jacket-connected tie-lines and anatomical prostheses such as shown in FIG. 36A are meant for interposition along a vessel to replace a defective segment, vascular valve-connected confluence conduits are meant to allow the grouping of tiny vessels in an infant, for example, to facilitate completion of a compound vascular heart transplant as addressed above in section 2b(1)(b)(b14), entitled Confluence Conduits: Connection of One or More Bloodlines to Flow through a Common Channel to the Same or a Different Number of Bloodlines with Self-leveling Apportionment of Outflow, in the subsection thereof entitled Tiny Vessel Grouped Inlet/Outlet Expandable Confluence Conduits, as well as in this section, and shown in FIGS. 15C, 15D, 36A, and 36F.

Another purpose for confluence conduits is to facilitate connection of a number of input vessels to a different number of output vessels as is often found in the renal vessels during a renal transplant, for example, with anomalous supernumerary or accessory branches where embolization is not preferred for reasons specified above in section 2b(1)(b)(b14), entitled Confluence Conduits: Connection of One or More Bloodlines to Flow through a Common Channel to the Same or a Different Number of Bloodlines with Self-leveling Apportionment of Outflow, in the subsection thereof entitled Risks Involved in the Surgical Embolization of Vessels. These fundamental differences considered, it should never be necessary to place a confluence conduit with inline coupling jackets using the mechanism depicted in FIG. 37A.

As do all side-entry and inline coupling components, one or more druglines such as that labelled 8, for example, make it possible for a totally implanted prosthetic disorder response system to detect the need for and release medication or prosthesis maintenance agents into the replacement segment at any level along the totality of its length to include its inline connectors. Such drugs and agents can simulate endothelial function or be used to dissolve a biofilm deposited about the endothelium or a crystalline accretion deposited about the urothelium, for example.

Of the four longitudinally spaced vertical lines in FIG. 36A referring to the transectional views in the figures to follow, the cross-section shown in FIG. 36B shows a drugline 8 of which there may be more than one, that allows the fully implanted automatic disorder control system to directly pipe-target drugs on a programmed basis for wetting of the adventitia with medication, and multiple through-and-through apertures 40 through the jacket dissolution-counteractant coated foam 4 and outer casing 3 which cooperate with the foam to minimize if not eliminate the compression of the tiny blood vessels and nervelets coursing along, into, and out of the adventitia (omitted for clarity) to counteract the atherosclerotic degeneration that would otherwise promptly ensue (reference cited in U.S. Pat. No. 11,389,171, entitled Integrated System for the Infixion and Retrieval of Implants and U.S. Pat. No. 11,759,186, entitled Ductus Side-entry Jackets and Prosthetic Disorder Response Systems).

The vertical lines between the entry and exit half-jackets referring FIG. 36C which shows the substrate automatic ductus transection mechanism addressed above in section 2b(2)(c)(c2), entitled Automatic Insertion into a Ductus of an Inline Coupling Jacket-secured Bridging, or Tie-line and Its Replacement if Necessary, as well as shortly below and schematized in FIGS. 37E and 37F. The remaining vertical view reference line through the exit half-jacket cites FIG. 36D which shows multiple through-and-through apertures 40, the dissolution-counteractant coated foam 4, and the jacket outer casing 3.

Any segment to any length along any vessel for which repair or replacement is necessary to include the aorta just short of its termini to allow the space to position the automatic cutting mechanism depicted in FIG. 37A where the tie-line type prosthesis would be the length of the entire aorta as allowed for in FIG. 37B can be replaced, to include any along the great vessels. That replacement is substantially bloodless and an interruption in the flow of blood avoided makes the replacement of an unsurvivably malformed aorta far less risky and less complex to replace in a dying neonate. The alternating circumferential flats and expansion vaults configuration of the tie-line joining the inline coupling jacket at either end indicates that the prosthesis includes a growth accommodating fabric for one-time placement in a child, eliminating the need for future revision.

### Artificial Aorta and its Automatic Placement

A cross-sectional view of the arrangement of mechanism between the half-jackets within each inline coupling jacket to effect jacket closure and replacement of the native with a prosthetic segment is shown in FIG. 36C. FIGS. 37E and 37F provide schematic representations of the components comprising the drive train of the automatic transection mechanism from hand control to reciprocating razors 226. FIG. 37A shows the starting position for replacing a defective segment along a native ductus with a tie-line or tie-line type prosthesis.

FIGS. 37A thru 37C, 37E, and 37F depict the structure and function of inline coupling jackets to include the tie-line, or prosthetic ductus segment, inner and outer half-jackets automatic closing mechanism, and in FIG. 37A, the rotation of the prosthesis into position to take the place of the native structure such as the aortic arch. When a vertically extended tie-line type prosthesis such as the aortic neoarch shown in FIG. 36F is to be placed as shown in FIG. 37A so that the vertical component would interfere with rotation, the torsion spring at the center of the divided hinge 228 indicated at the center in FIG. 37C (not drawn in detail) must be strong enough to allow the rotation to be intermittently affected by the operator.

The mechanism positioned between the inlet and outlet coupling jacket half-jackets, or half-cylinders, with serrated razor edge 226 along one side and an undulating rippled trip retention line on the opposite side 229 to retain the trip arms thus preventing closure of the inner half-jackets under the urging of their spring loaded axles until released through dislodgement by the eccentric vibratory motors is shown in FIGS. 36C and 37C with schematic representations provided in FIG. 37E and one more pictorial in FIG. 37F. This is described above in section 2a, entitled Background of the Invention. General, as well as above in section 2b(2)(c)(c1), entitled Inline Coupling Jackets Proper, as well as in this section.

While not part of the drive train, the damped jacket closing spring hinges coaxial with the hinges connecting the jacket halves 228 cooperate with the drive train in closing the jackets to transect and replace the native with the synthetic ductus. To assure simultaneity of jacket closure, hence, ductus replacement with continuity of blood flow from the native through the prosthetic segment, control from the hand control of which the output is divided to the entry and exit jackets is unitary.

With all five prospective native stumps cinched about by the outer half-jackets actuated in synchrony from a common switch as shown in FIGS. 37E and 37F, the operator intermittently pushes against the three excessively extended neosupraaortic branches to allow these to simultaneously clear the available gap for rotation into the completely aligned position. To avoid a complete rotation where the branches are too short, some slight addition in length is harmless.

The native vessel transection mechanism for the automatic replacement of a defective segment along a major vessel is described above in section 2b(2)(c)(c2), entitled Automatic Insertion into a Ductus of an Inline Coupling Jacket-secured Bridging, or Tie-line and Its Replacement if Necessary, and is shown in FIGS. 37A, 37C, 37E, and 37F. The mechanism has its most frequent application to the thoracic aorta which is subject to numerous congenital malformities and incisions, for example, but is no less applicable to other stretches along a larger vessel such as the abdominal aorta where it is heavily affected by atheromatous plaque, an aneurysm, stenosis, or any other defect.

Due to number of branches, for replacement in a single operation, it is best to divide the length of an especially long tie-line type prosthesis such as the abdominal aorta into shorter intermittent segments as to be interposed at intervals by healthy native segments. For placement in a neonate or infant, as well as virtually any prosthetic replacement all segments must exhibit substantially the same growability as the corresponding native vessel. To this end, the thickness and plasticizer content, hence, pliancy, of the material made of the expandable fabric described above in section 2b(2)(f), entitled Expandable Structure to Accommodate Growth and Self-adjust in Size in Vascular Segment Prostheses, Lines, and Confluence Conduits Placed in Children, is adjusted.

Replacement thus is always preferable to the conventional placement of a patch aortoplasty in a pediatric patient as the prosthetic segment placed is expandable to accommodate growth and superior in any patient with a connective tissue disorder as might lead to any of numerous complications following a conventional repair. Curved, crowded together with the other great vessels, and directly receiving the output of the left ventricle, the thoracic aorta is subject to the highest pressures in the circulatory system.

Moreover, these challenges to providing a durable and dependable prosthesis to replace a segment along a major vessel additionally comprehend a complex interrelation of materials properties, physiology, and manufacturing technology. For these reasons, to devise a safe and dependable artificial aorta, much less one that will expand to accommodate growth and propagate the pulse, is more challenging than to devise a replacement abdominal aorta. Along with more difficult but still feasible repairs to the congenitally malformed heart, it is one of the most challenging problems posed by the vascular tree.

Accordingly, if a successful replacement thoracic aorta with branches can be produced, to provide a replacement for a severely affected segment along the abdominal aorta-even one that includes the celiac axis, the mesenterics, and the common iliac-would be readily provided. Accordingly, use of the thoracic aorta is exemplary. Such a prosthesis must securely connect to the cut ends of the native vessel, expand and contract to propagate the pulse, never leak, and to avert the need for replacement with the trauma of surgery, even in a prenate or neonate, should expand with growth for years if not life. Connection to the cut ends of the vessel is by the inline coupling jackets which also perform the replacement of the segment under the control of the prosthetic disorder response system.

The need for a fabric intended to span the distance between the valve-jacket at either end and satisfy the stringent requirements posed by a prenate, neonate, or infant was addressed above in sections 2a on general background and 2b(2)(f), entitled Expandable Structure to Accommodate Growth and Self-adjust in Size in Vascular Segment Prostheses, Lines, and Confluence Conduits Placed in Children. Essentially, conventional repairs such as a Dacron® (DuPont deNemours, Inc., Wilmington, Delaware) polyethylene terephthalate patch aortoplasty lack durability and the ability to expand with growth. These are untenable incapabilities in a prosthesis for a small child that unable to expand would require replacement in any event, and were it to fail would almost certainly result in death. This in a patient with a congenital connective tissue disorder likely to produce additional defects to require revision.

While antimicrobials can be released into the lumen, the outer surface of these materials may be intrinsically biocidal or made so through readily accomplished coating (see, for example, Badawy, H. T., Brunelliere, J., Veryaskina, M., Brotons, G., Sable, S. and 8 others 2015. “Assessing the Antimicrobial Activity of Polyisoprene Based Surfaces,” Online, MDPI [Multidisciplinary Digital Publishing Institute] International Journal of Molecular Sciences (Basel, Switzerland) 16(3): 4392-4415; Badawy, H. T., Pasetto, P., Mouget, J. L., Pilard, J. F., Cutright T. J., amd Milsted, A. 2013. “Bacterial Adhesion and Growth Reduction by Novel Rubber-derived Oligomers,” Biochemical and Biophysical Research Communications 438(4): 10.1016/j.bbrc2013.07.120; Jellali, R., Campistron, I., Pasetto, P., Laguerre, A., Gohier, F. and 3 others 2013. “Antifouling Activity of Novel Polyisoprene-based Coatings Made from Photocurable Natural Rubber Derived Oligomers,” Progress in Organic Coatings,” 76(9): 1203-1214; Kebir, N., Campistron, I., Laguerre, A., Pilard, J.-F., Bunel, C., and Jouenne, T. 2007. “Use of Telechelic Cis-1,4-polyisoprene Cationomers in the Synthesis of Antibacterial Ionic Polyurethanes and Copolyurethanes Bearing Ammonium Groups,” Biomaterials (Amsterdam, North Holland, Netherlands) 28(29): 4200-4208).

In FIG. 37A, the control wire to each jacket terminates in a pin plug which is inserted into a small socket positioned in line with the razors on the outside of either jacket before the control switch is depressed to start both pin plugs, hence, both razor saws, reciprocating. Because the operator or an assistant can observe the process directly, or if performed endoscopically, with the aid of ultrasound, to fully automate jacket closure is discounted as adding needless expense.

Also, in FIG. 37A, control switch 215 is mounted atop hand control 216 powered by inmate batteries in battery compartment 217. To facilitate the routine maintenance of the control for immediate use at full charge, the batteries are preferably rechargeable, and to avoid distraction due to excessive weight, of the small button cell type. While FIG. 37A shows the components of the entry jacket, labeled as part number 195, in FIG. 36E, for example, exit jacket 195′ should be understood as identical, differing only in being reversed in direction.

A common power source for both entry and exit jacket razors is the initial factor essential to assure simultaneity of transection at either end of the segment along the vessel to be replaced and therefore to allow the uninterrupted passage of blood during segment replacement. Hand control 216 is connected by electrical conductor 218 (for example, a vibration transmission line) to both cell phone type vibration motors 219 used to drive the cutting action in the outlet half of the entry jacket 200 and the inlet half of the exit jacket 200′ through reciprocation, not electrically conducting phono-sized pin plugs, or pin jacks 220, mounted at the front of motors 219.

Spring-loaded to reciprocate, the razor mountings extend radially outward to a socket key inset at the jacket periphery into which pin jacks connected to a button cell-powered cellular telephone type vibratory motor can be inserted. Shown in FIGS. 36A, 36E, and 37D, the outlet half of the entry jacket 200 and inlet half of the exit jacket 200′ are kept from descending onto the native vessel by trips 223 that drop down to engage wave, or undulating depressions 229 along the upper edge of serrated, or saw-toothed, razor 226 until pin jacks 220 are both inserted and the cutting razors energized.

Conjunction of the front ends of pin plugs 220 against the driven abaxial end of razor reciprocating saws 226 is maintained by tiny neodymium iron boron permanent magnets 222 bonded to the free ends of pin plugs 220. Razor reciprocating saws 226 ride upon thin sheet steel sliding back mounting stages 227, set upon slideways, which positioned between the forcibly reciprocating front ends of pin plugs, 220 and compression springs 225, cause the razors to reciprocate at high speed when control switch 215 mounted atop hand control 216 is depressed sending current to motors 219.

FIGS. 36C and 37C show the transection mechanism at the interface between the inlet and outlet jacket halves used to effectuate the connection between the native and synthetic lines under automatic control to reconstitute a ductal continuum. FIGS. 37E and 37F provide schematized representations of the automatic transection mechanism in the context of the control system as a whole wherein the latter is somewhat more pictorial. Having transected the native segment and rotated its synthetic replacement into alignment with the native stumps, the transecting and prosthesis positioning halves of the jackets—the outlet half of the entry jacket 200 and the inlet half of the exit jacket 200′—finally close under the urging of the damped jacket spring hinges 228 and are automatically locked shut by the small latch and retainer type lock of the pawl-intromissive types seen on briefcases but much smaller.

Shown in FIG. 37B, suspended to swing freely from the periphery of the jacket above and behind therefore to drop past replacement prosthetic vessel segment 198 when the outlet half of the entry jacket 200 and inlet half of the exit jacket 200′ is drop-kick trip bar, or simply trip. 223 devised for the utmost simplicity to consist of a small bar with rounded free lower end rendered slippery by burnishing or a fluoropolymeric coating such as polytetrafluoroethylene (poly(1,1,2,2-tetrafluoroethylene).

Accordingly, when the vertically oriented jacket is opened, drop-kick trip bar 223 drops down to hang behind replacement prosthetic vessel segment 198, so that when the jacket is released, the restorative force of the jacket spring hinges 228 causes the rounded tip of drop-kick trip bar 223 to enter into and become lodged within one of the depressions 229 in a continuous train of undulating depressions 229 along the upper surface of razor reciprocating saws 226 over the length of the upper surface of reciprocating razor saws onto which trip preventing further closure of the jacket until trip 223 is dislodged.

The depressions 229, which need not extend over more of the razor length than the drip would contact, are so configured that an abrupt motion of serrated razor saws 226 in their central linear axis will dislodge the trips and the quick reciprocation of these from side to side will not allow the trip 223 to reengage in any of the depressions 229, so that the entry and exit jackets continue to close until the diseased or malformed ductus-usually a larger vessel—is cut through and its prosthetic replacement rotated into its position.

Insertion of electrically nonconducting pin plugs 220 into pin plug sockets 221 causes magnets 222 bonded to the tips of pin plugs 220 to seize and hold the abaxial (front, proximal) end of razor reciprocating saws 226 back mounting stages 227 to the front ends of pin plugs 220, so that when driven by vibrating motors 219, pin plugs 220 push against the front end of back mounting stages 227, the restorative force of tiny compression springs 225 at the opposite, rear end of back mounting stages 227 cause back mounting reciprocating stages 227 to rebound razor reciprocating saws 226 back toward pin plugs 220.

Accordingly, when vibratory motors 219 are energized, the following sequence of actions occurs simultaneously at either end of the native segment to be replaced:
1. Pin plugs 220 drive razor reciprocating saws 226 back mounting stages 227 backwards, or axially, and compression springs 225 at the opposite, rear end of back mounting stages 227 then driving razor reciprocating saws 226 back mounting stages 227 back to the fore, or abaxially, so that razor reciprocating saws 226 are made to reciprocate at a high rate causing
2. Trip 223 to be dislodged from the depression 229 in an undulating, or rippled, train thereof running along the upper side of razor mounting stage 227 of such depressions 229, along the length of the upper surface of reciprocating razor saws 226 where the free end of trip 223 so that
3. Damped jacket spring hinges 228 have been released to cause reciprocating razor saws 226 to descend upon and instantly transect the segment of the native vessel at either end, whereupon
4. Continued closure of the jacket half will then have rotated replacement prosthetic segment 198 into the position previously occupied by the native segment with the junction at either end bleeding little if at all, as well as have locked and firmly secured the jacket halves in the locked position.

Summarizing, depressing switch 215 on manual control 216 sends current to vibrating motors 219 dislodging trips 223 from the depression in the rippled train of such depressions 229 along the upper surface of reciprocating razor 226 into which either became engaged, causing pin plugs 220 to reciprocate against compression spring 225 and driving compression spring-loaded razor mounting track 226 to reciprocate the razors, allowing the downward driving restorative force of the jacket spring hinges 228 to continue closure of both entry 200 and exit 200′ jacket halves.

The jackets therefore bring the reciprocating razors down upon the native ductus simultaneously effecting its transection at either end, after which continued rotation under the restorative force of jacket spring hinges 228 aligns replacement prosthetic vessel segment 198 in the same position as had been originally occupied by the native vessel. This simultaneity at either end of the replacement segment minimizes if not eliminates any perturbation in the flow of blood that might otherwise have induced a cerebral or cardiac accident while the prosthesis rotated into position.

Because the dislodging of trip 223 is simultaneous with the initiation of reciprocation and continued closure of the jacket, once dislodged, trip 223 glances off any prominence it contacts sideways without the ability to engage another depression, the slippery tip of trip 223 not allowing trip 223 to become stuck anyway. Transection and replacement of the native by replacement prosthetic vessel segment 198 follows simultaneously and without interruption at both the entry and exit jackets. In use, the inline coupling jacket is pulled open against the jacket closing restorative force of the highly damped spring hinges 228 along the common axle-joining the inlet and outlet halves, so that the junction between the native vessel and the prosthetic bloodline is in line with the junction between the jacket inlet and outlet halves.

Along the thoracic aorta, this typically includes an entry jacket at the proximal terminus along the ascending aorta of the segment to be replaced, exit jackets on each of the supra-aortic branches (brachiocephalic trunk (innominate artery), left common carotid artery, left subclavian artery) and an exit jacket the same in size as the entry jacket with its transection razor mechanism positioned at the level of the aorta where the prosthetic replacement segment is to terminate. This sets the levels at which the defective or diseased native vessel will be transected and the native and synthetic ends in adjoining relation, sealed continuity between the two obtained by making the synthetic end slightly protrude into touching contact with that native.

As shown in FIG. 37A, exit and entry jackets are structurally and functionally identical except oriented in the opposite directions. In FIG. 37A, part number 230 represents the vascular prosthesis. 231 and 231′ the positions of the native vessel transection devices, 228 and 228′ jeweled bearings between the inner and outer half-jackets, and the arrows indicate the rotation of the prosthesis 230 into the position previously occupied by the segment of the native vessel replaced. Simple tubular bloodline tie-lines, confluence conduits as arterial supply and venous return arms such as those shown in FIGS. 15A and 15B, and tiny vessel grouped or gathered inlet/outlet expandably structured confluence conduits as arterial supply and venous return arms such as those shown in FIG. 15C are marked as part numbers 230 if arterial and 196′ if venous.

Bloodlines to include anatomically shaped tie-line bloodlines such as a prosthetic thoracic aorta or aortic arch as shown in FIG. 36F for replacement of a defective segment along an arterial or venous vessel are assigned part number 196 if arterial or 196′ if venous. Arterial confluence conduits are assigned part number 230. Venous return to the heart consisting of the two large venae cavae, each is a bloodline 196′: In FIG. 37A, while shown as simply tubular, part number 196 is meant to represent a tie-line type prosthesis such as that shown in FIG. 36F.

As seen in FIG. 37A, inlet and outlet native vessel transection mechanisms 231 and 231′, which firmly fix the transection mechanisms in the vertical position as the spring-loaded axles once tripped rotate the inner half-jackets and serrated transection razors 229 as the arrows indicate are connected to the ends of either axle rotational split end joint by jeweled bearings. Half jacket joining axles 228 and 228′ bearings are schematically shown end-on in FIGS. 36C and 37C. Referring to FIGS. 37A, 37E, and 37F, the rotation into alignment with the stumps of the defective native segment by the inner half-jackets 200 and 200′ under the restorative force of their respective spring-loaded axles is initiated by inserting phono jack-like miniature pins 220 into pin sockets 221 as shown in FIGS. 37D and 37E.

### Endothelial Function and Epithelialization of the Bloodline or Tie-Line Type Vascular Prostheses

Except for the reciprocating saw-like serrated razors 226 and 226′ in FIGS. 37D and 37E, inline coupling jacket transection mechanisms, part numbers 231 and 231′ shown or indicated schematically in FIGS. 36C, 37C and 37A can be made of a low friction polymer such as a gear grade nylon, polytetrafluoroethylene, or polyetheretherketone. However, as to the bulk of the material of the prosthesis, drug uptake by the endothelium addressed above in section 2b(1)(b)(b1), entitled Endothelial Drumming, as well as the secretory and other functions of endothelium cited at various points in this specification might justify the use of alternative materials which allow quicker epithelialization with reduced intimal proliferation along the internal surface of the tubular tie-line or anatomically shaped vascular prosthesis.

Depending upon the significance of endothelial development for endothelial function to line the internal walls of a synthetic vascular segment to replace that native-which unlike the native vessel transection devices 231 and 231′ are exposed to the bloodstream—may best be made of a porous biodegradation-resistant polycarbonate polyurethane or another synthetic material less susceptible to stenosis and graft thrombosis (see, for example, Jeschke, M. G., Hermanutz, V., Wolf. S. E., and Koveker, G. B. 1999. “Polyurethane Vascular Prostheses Decreases Neointimal Formation Compared with Expanded Polytetrafluoroethylene,” Journal of Vascular Surgery (New York, New York) 29(1): 168-176; see also, for example, Abyzova, E., Dogadina, E., Rodriguez, R. D., Petrov. I., Kolesnikova, Y., and 3 others 2023. ““Beyond Tissue Replacement: The Emerging Role of Smart Implants in Healthcare,” Online, Materials Today. Bio (London, England) 22:100784; Ding, C., Ma. J., Teng. Y., and Chen, S. 2023. “The Effect of Plasma Treatment on the Mechanical and Biological Properties of Polyurethane Artificial Blood Vessel,” Online, MDPI [Multidisciplinary Digital Publishing Institute] Materials (Basel, Switzerland) 16(22): 7231; Liu, C., Dai, J., Wang, X., and Hu X. 2023. “The Influence of Textile Structure Characteristics on the Performance of Artificial Blood Vessels,” Online, MDPI [Multidisciplinary Digital Publishing Institute] Polymers (Basel, Switzerland) 15(14): 3003; Fathi-Karkan, S., Banimohamad-Shotorbani, B., Saghati, S., Rahbarghazi, R., and Davaran, S. 2022. “A Critical Review of Fibrous Polyurethane-based Vascular Tissue Engineering Scaffolds,” Online, [BioMed Central] Journal of Biological Engineering,” (London, England) 16(1): 6; Guo, H. F., Dai, W. W., Qian, D. H., Qin, Z. X., Lei, Y., Hou, X. Y., and Wen, C. 2017. “A Simply Prepared Small-diameter Artificial Blood Vessel that Promotes in Situ Endothelialization,” Acta Biomaterialia (Kidington, Oxford, England) 54:107-116; Desai M. and Hamilton, G. 2011. “Graft Materials Past and Future,” Chapter 28 in Fitridge, R. and Thompson, M. (eds), Mechanisms of Vascular Disease: A Reference Book for Vascular Specialists, Online, Adelaide, South Australia: University of Adelaide Press).

While a synthetic prosthesis is itself unsusceptible to infection or metaplastic transition, an endothelial layer that develops along its internal walls is not, and as to later term implications, by derivation and composition, endothelium may be more susceptible to clot and differing from nonendothelial epithelium, undergo mesenchymal metaplastic fibrotic malignant transition—an important consideration for use in synthetic lines and confluence conduits for placement in pediatric patients (see, for example, Cheng, W., Li, X., Liu, D., Cui. C., and Wang, X. 2021. “Endothelial-to-Mesenchymal Transition: Role in Cardiac Fibrosis,” Online, Journal of Cardiovascular Pharmacology and Therapeutics (Thousand Oaks, California) 26(1): 3-11; Piera-Velazquez, S. and Jimenez, S. A. 2019. “Endothelial to Mesenchymal Transition: Role in Physiology and in the Pathogenesis of Human Diseases,” Online, Physiological Reviews (Bethesda, Maryland) 99(2): 1281-1324; Kovacic, J. C., Mercader, N., Torres, M., Boehm, M., and Fuster, V. 2012. “Epithelial-to-Mesenchymal and Endothelial-to-Mesenchymal Transition: From Cardiovascular Development to Disease,” Online, Circulation (Hagerstown, Maryland) 125(14): 1795-1808).

More specifically, this factor is especially pertinent with respect to the expandably structured lines and confluence conduits for use in the young and the very young as delineated above in section 2b(2)(f), entitled Expandable Structure to Accommodate Growth and Self-adjust in Size in Vascular Segment Prostheses, Lines, and Confluence Conduits Placed in Children, with respect in particular to grouped vessel inlet/outlet confluence conduits as shown in FIGS. 15C and 15D and addressed above in section 2b(1)(b)(b14), entitled Confluence Conduits: Connection of One of More Bloodlines to Flow through a Common Channel to the Same or a Different Number of Bloodlines with Self-leveling Apportionment of Outflow, in the subsection thereof entitled Tiny Vessel Grouped Inlet/Outlet Expandable Confluence Conduits, as well as in this section.

Until substantially extended, the domes in expandable lines and confluence conduits will likely disrupt laminar flow, although no more so than would flow past the ostia of branches anywhere else in the vascular tree. For the synthetic ends of the vascular prosthesis as replacement segment to slide over and become engaged within the stumps of the native vessel proximate to the surface of the inline coupling outer half jackets as shown in in FIG. 37A, the ends of the prosthesis are made to slightly overextend past the entry surface of the outer half jackets, are feathered, and resilient with the surfaces of both ends prepared with a slippery coating to minimize sliding friction.

The automatic insertion of a tubular tie-line or anatomical prosthesis to replace a segment along a native vessel is delineated above in section 2b(2)(c)(c2), entitled Automatic Insertion into a Ductus of an Inline Coupling Jacket-secured Bridging, or Tie-line and Its Replacement If Necessary. Referring to FIGS. 37D and 37E, initiating jacket closing is by inserting phono jack-like pins 220 and 220′ (shown as singular) into phono jack-like pin sockets 221 and 221′ at either end of the segment to be replaced.

An option shown in FIG. 37E when retention of jack-like pins 220 and 220′ in sockets 221 and 221′ based upon pin conformation alone is not fully dependable is to provide the distal tips of each pin with a neodymium iron boron magnet. Because the cutting action of miniature serrated razor reciprocating saw does not begin until eccentric vibratory motors 219 have been actuated, insertion of pins 220 and 220′ in sockets 221 and 221′ does not in itself initiate dislodgement of trips 223 and 223′ [the latter not shown] freeing the spring-loaded half-jacket axles to close the half-jackets. This affords the operator an interval during which the procedure up to this point can be carefully reviewed.

In the drawing figures, arterial bloodlines are marked 196, those venous 196′, arterial confluence conduits 230, and (venous drainage limited to the superior and inferior venae cavae, azygos vein, and pulmonary ‘arteries’) so that venous confluence conduits are hypothetical, 230′. The prosthesis insertion action is initiated at both ends of replacement prosthetic segment 230 in FIGS. 37A and 37B from a single switch 215 atop hand control 216 shown in FIG. 37E. Where two part numbers appear, the vessel conforms to both descriptions. Any additional figure numbers applied to a given line pertain to a specific function such as a long-to-short switched line.

Referring to FIGS. 36A, 36E, 37A, and 37D, the inline jackets as a whole—to include both entry inlet, or outer, half-jackets 199, and 200 and corresponding but reversed exit half-jackets 200′ and 199′—are labeled 195 and 195′ at either end of the segment to be replaced. Insertion of miniature phone jack-like pins, part numbers 220 and 220′ [the latter not shown] in FIGS. 37E and 37F does not in itself initiate the dislodgement of trips 223 and 223′ to initiate the cutting action, so that both need not be inserted at the same moment, which pause affords the operator an interval to review the procedure. Small phono jack-like pins, or pin plugs, 220 [its antipode 220′ not shown] are inserted into pin jack sockets 221 [its antipode 221′ not shown] in FIGS. 37D and 37E.

This actuates miniature vibratory DC eccentric rotor brush motors 219 and 219′ causing trips 223 and 223′ to be released from their depressions in the rippled strips 229 and 229′opposite the serrated razors 226 and 226′, freeing the spring-loaded half-jacket axles to close down upon the native ductus so that the serrated razors 226 and 226′ effectuate the cutting action. The native ductus is simultaneously transected at either end at the levels where the inlet and outlet halves of the entry and exit jackets meet. The replacement action places the tops of reciprocating razors 226 and 226′ just beneath the cuts they have executed with the ends of conduit 198 aligned to and engaged within the ends of the native ductus stumps.

The transection and replacement action completed, to eliminate any possibility for reentry of any part of the native vessel transection mechanisms-part numbers 231 and 231′ in FIGS. 36C, 36E, 37A, and 37C thru 37E to back up into the native-prosthetic-native lumen once the rotation indicated in FIG. 37A has been completed which would disrupt the primarily laminar, or streamline, 228 and 228′ flow of blood and possibly accumulate clot, a mounting backplate, even one consisting of only a minimized frame with thin arms, has been eliminated. Instead, to the vertically aligning force of the tight jeweled bearings in the spring-loaded axles are added spring-loaded catches to lock the serrated reciprocating saw-like razors 229 and 229′ in place at the end of their throws.

There must be no difference in the instant the trips are dislodged from their depressions and the cutting action begun at either end. Therefore, mere insertion of the pins before razor reciprocation is switched on must not disengage the trips. Specifically, withdrawal of the trips upon insertion of the pins before the cutting action is switched on would introduce a variable interval during which the razors could cut into the substrate native ductus causing bleeding before the jackets closed about the ductus to seal off any outflow of blood. Accordingly, both to assure that transection will start at the same instant and that no premature incision can occur at either end, retraction of the trips and the initiation of the reciprocating action must occur simultaneously. That is, the reciprocating action both retracts the trips and transects the native ductus.

Thereafter, using switch 215 atop manual control 216 by the operator or an assistant simultaneously retracts the trips and initiates the cutting action. To prevent extravasation that were it to continue for more than a few seconds could provoke a serious cardiac or vascular accident, transection of the native ductus at either end must be simultaneous. For this reason, it is important that insertion of the pins not retract the trips in a separate preliminary step before the razors are energized. Transparent or translucent materials used, any malfunction in this sequence will be immediately apparent as a failure in the rapid transfer of blood flow from the native into and through the prosthetic ductus as the razors advance.

Carefully manufactured and tested, inline coupling jackets should never malfunction. Nevertheless, where there is some trepidation, ‘bailout’ measures at a distance from the brain or the heart consist of clamping followed by withdrawal of the tie-line or prosthesis and the repair of whatever damage had resulted by the time the malfunction occurred. Another tie-line or prosthesis longer than the original so that the synthetic segment will cover over the injured parts is then used. Near to the brain or the heart where the flow of blood must not be interrupted, a bypass connected upstream to the entry jacket and downstream to the exit jacket is best connected with vascular valves.

At the levels where the valves are to be connected, the substrate adventitia is wetted with a lubricant such as ACS Microslide®, Medtronic Enhance®, Bard Pro/Pel®, Hydro/Pel®, Cordis SLX®, of Rotaglide®. After repair, the trepans are retracted, the valves slid aside from the trepan openings, immediately covered over with the thumb, and patched. Inserting cardiopulmonary bypass cannulae—not larger extracorporeal membrane oxygenation cannulae-prior to placement of the tie-line is not preferred as more injurious and extending invasiveness to remote sites. The highly damped spring hinge inner half cylinder jackets 224 and 224′ in FIGS. 36A and 36F at the rear of only the inner half cylinders along this common axle or spine urges the outlet half to gently but forcibly close when opened.

Referring to FIGS. 37E and 37F, the retraction of the trips of the inner, or transection halves, that is, of the entry jacket 200 and the inlet half of the exit jacket 200′ and actuation of the cutting razors 226 upon insertion of the pin jacks:
1. Frees the highly damped spring hinges 224 and 224′ shown in FIGS. 37E and 37F but to allow clarity only positionally indicated in FIG. 36A to continue the descent onto the native ductus of reciprocating serrated razors 226 shown in FIGS. 36C, 37C, 37E, and 37F.
2. Transect the native ductus at either end while rotating the synthetic replacement segment into alignment with the native stumps.

The slightly extended ends of conduit 198 therefore enter into the receiving lumen holes of the entry and exit inline coupling jackets to securely engage the ends of the vascular segment prosthesis which in an adult is essentially tubular, and in a child, made with the expandable structure. To assure a good end-to-end fit, or ‘anastomosis,’ between the native and synthetic ductus at the interfaces, both are made round; however, the interfaces are surrounded with foam and the prosthetic segment is compliant in cross-section. Compliant moving away from the interface, both will spontaneously flatten sufficiently to accommodate the neighboring anatomy. This gradual change in cross-sectional contour should induce little turbulence to detract from laminar, or streamline, flow.

The rubbery ends of conduit 198 and the sides of the hole therefore form a seal much like O-rings to enclose the conduit-ductus joints, sealing these off to substantially if not completely eliminate any outflow of blood at the joints. Additional means for securely sealing and inseparably joining the jacket half junctions are specified above in sections 2b(2)(c), entitled Vascular Segment Prostheses: Inline Coupling Jackets and Bridging, or Tie-lines. Fully closed, the outlet half of the entry jacket, for example, is locked shut by the intromission of a small L-shaped pawl retained within one of the small locks used to secure all of the half-jackets by a spring-loaded undercutting arm, which type lock is simply a miniature version of those seen on brief cases, for example.

Controlled by the implanted disorder response system, an inlet partial flow diversion servovalve with a thin film pressure sensor in a prosthetic aortic arch implanted to replace one native that had been aneurysmal or coarcted, for example, can reduce the blood pressure by passing a fraction of the blood into a large neighboring vessel where the additional volume would be proportionally less and likely harmless, such as the superior vena cava. Hardly alien to the circulatory system, valves are a part of every more highly developed closed circulatory system to include those of cephalopods and vertebrates.

Essential to support collateral development, the heart of the human embryo must provide circulation at three weeks into gestation. As development progresses, the elaboration and differentiation in the structures to be supported is reflected in the coordinated elaboration and differentiation of the heart. At nine weeks-which marks the transition from the embryonic to the fetal stage in development—there arises a need for tighter control over the flow of blood between the emerging chambers of the heart and great vessels.

The primordia, or anlagen, of the heart valves appear at eight weeks, and by nine, the control of flow under pressure is mediated by valves and a nascent conduction system. The importance of normal vascular as well as cardiac valvular function is epitomized in the birth process, where the transfer of oxygenation from the placenta to the lungs requires the closure of four ducts—the ductus arteriosus, the ductus venosus, the foramen ovale, and the placenta itself—which by closing (or in the case of the foramen ovale, considerably shrinking in size), effectively function as one-time valves.

Valves are essential from the beginning of life—to develop from the embryo into a zygote, then a fetus, to be separated from a parasitic status in relation to the mother, that is, born as a physiologically independent organism, and thereafter, to not only attain but remain alive. The replacement of a congenitally defective great vessel such as the thoracic aorta encouraged to be replaced upon detection or as soon thereafter as practicable in accordance with section 2b(2)(c) above, entitled Vascular Segment Prostheses: Inline Coupling Jackets and Bridging, or Tie-lines, and its subordinate subsections, the development of baroreflex sensitivity is a consideration.

Even as a neonate, the flow of blood is also controlled by pressure sensing chemo- and baroreceptors that signal the need to adjust the pumping action of the heart and to secrete vasodilators or vasoconstrictors (Yiallourou, S. R., Sands, S. A., Walker, A. M., and Horne, R. S. 2010. “Postnatal Development of Baroreflex Sensitivity in Infancy,” Online, Journal of Physiology (London, England) 588 (Part 12): 2193-2203; Andriessen, P., Oetomo, S. B., Peters, C., Vermeulen, B., Wijn, P. F., and Blanco, C. E. 2005. “Baroreceptor Reflex Sensitivity in Human Neonates: The Effect of Postmenstrual Age,” Online, Journal of Physiology (London, England) 568 (Part 1): 333-341; see also Javorka, K., Hašková, K., Czippelová, B., Zibolen M., and Javorka, M. 2021. “Baroreflex Sensitivity and Blood Pressure in Premature Infants—Dependence on Gestational Age, Postnatal Age and Sex,” Online, Physiological Research (Czech Republic) 70 (S3): S349-S356; Javorka, K., Haskova, K., Czippelova, B., Zibolen, M., and Javorka, M. 2021. “Blood Pressure Variability and Baroreflex Sensitivity in Premature Newborns—An Effect of Postconceptional and Gestational Age,” Online, Frontiers in Pediatrics (Lausanne, Switzerland) 9:653573).

As attested to by the urethral and anal internal and external sphincters and the sphincters along the gastrointestinal tract, valves are hardly limited to the circulatory system. Urinary continence is imparted by the internal and external urethral sphincters and fecal continence by the internal and external anal sphincters-essentially valves, and along the gastrointestinal tract where the contents are semisolid and successive segments absorb different nutrients, sphincters serve as valves demarcating the segments. The lymphatic system is replete with valves, and the irises of the eyes are valves responsive to the intensity of light.

Application of the term ‘valves’ to the unidirectional guides which support the return of blood to the right atrium when compressed by the calf muscles are properly understood to exert a mechanical pumping function from the lower legs upwards against the force of gravity. Providing a valving function, these are properly referred to as valves. In terms of the technical definition of a valve, simple one-, two-, and three-way fixed, that is, nonadjustable, side-entry jacket flow directional guides as shown in FIGS. 10F thru 10H, addressed above in section 2b, entitled Vascular Valves and Servovalves in the Diversion of Blood Flow, in the subsection thereof entitled One-, Two-, and Three-way Passive Side-entry Flow Diversion Jackets are not valves, but may be necessary for use with valves to direct blood flow as shown in FIG. 15B, for example.

What is claimed is:

1. A perivascular jacket for encircling a native vessel, said perivascular jacket having bonded to it a tubular outlet passageway defining a lumen continuous with that of said native vessel,
wherein said tubular outlet passageway extends perpendicularly from said perivascular jacket such that said perivascular jacket can be positioned selectively at any anatomically available level and rotational angle,
wherein a valve is disposed within the tubular outlet passageway, said valve comprising a controllably-driven native lumen intromissive tongue, the controllably-driven native lumen intromissive tongue comprising an arm extending a controllable distance into a lumen of said native vessel,
wherein a distal end of the arm comprises an upturned distal edge, thereby serving as a valve to draw away or modulate the flow of a volume through said native vessel into said tubular outlet passageway for discharge out of the perivascular jacket through said tubular outlet passageway.

2. The perivascular jacket according to claim 1, wherein extension and retraction of said native lumen intromissive tongue is driven by a servomotor configured to provide continuously variable control of the extension and retraction of said native lumen intromissive tongue.

3. The perivascular jacket according to claim 1, wherein extension and retraction of said native lumen intromissive tongue is driven by a solenoid.

4. The perivascular jacket according to claim 1, wherein said distal tip of the controllably-driven native lumen intromissive tongue is distally bifid and bidirectional with both craniad and caudad outlets.

5. The perivascular jacket according to claim 1, wherein the controllably-driven native lumen intromissive tongue is conformed to accept flow from multiple vessels in parallel adjacency such as those encountered in a neonatal heart transplant.

6. A perivascular jacket selectively positionable along and about a tubular anatomical structure for delivering drugs, passing cabled devices into, and diverting a measured portion of the luminal contents and biopsy tissue samples a lumen of said tubular anatomical structure, the perivascular jacket comprising:
an outer shell comprising semicylindrical halves joined together along a common edge so as to form a plano-type hinge so that, when opened and placed to encircle the said tubular anatomical structure, the semicylindrical halves grip about said tubular anatomical structure as a collar;
wherein a cushioning layer configured to protect small nerves and vessels that enter and depart from an outer surface of said tubular anatomical structure lines an internal surface of each of said semicylindrical halves of the outer shell,
wherein perforations pass entirely through said outer shell and said cushioning layer are placed to give access to the lumen of said tubular anatomical structure,
wherein an opening is defined in the side of said collar into which a side tube with a trepan front edge can be inserted, wherein said side tube is rotatable around and reciprocable along a longitudinal axis of the side tube thereby facilitating excision of a plug of tissue from a wall of said tubular anatomical structure so as to form a side opening in the tubular anatomical structure such that a lumen of said side tube will be continuous with the lumen of said tubular anatomical structure, and
wherein said side tube is fixable in rotational angle and depth of penetration into a side of said tubular anatomical structure,
wherein a self-locking screw-down cap with internal expansion bushing that fits onto an external thread at the base of said side tube allows said side tube to be fixed in rotational angle and depth of insertion into said side opening of the tubular anatomical structure; and
a projectable and retractable tongue-shaped diversion chute with an upturned distal end mounted within said side tube and configured to slide reciprocally to selectively positional depths into the lumen of the said tubular anatomical structure so that a column of bodily fluid upstream of the diversion chute is diverted out through said side opening and said side tube so as to provide continued flow through a synthetic tube,
wherein the perivascular jacket comprises a vascular valve.

7. The perivascular jacket according to claim 6, wherein the vascular valve comprises a permanent magnet layer along the internal surface of the semicylindrical halves of said outer shell;
wherein said magnet layer is disposed between said outer shell and said cushioning layer, wherein the magnet layer is interrupted to accommodate the opening and closing of said collar and the passing through of said perforations,
wherein said magnet layer is magnetized to exert an attractive force centrally toward and perpendicular to a longitudinal axis of said collar, making possible the detention and extraction of magnetically susceptible luminal contents.

8. The perivascular jacket according to claim 6, wherein the vascular valve comprises a plurality of electromagnets disposed between said outer shell and said cushioning layer,
wherein the plurality of electromagnets are interrupted to accommodate the opening and closing of said collar and the passing through of said perforations,
wherein said plurality of electromagnets are selectively energizable to exert an attractive force eccentrically and collectively energizable to exert an attractive force centrally toward and perpendicular to a longitudinal axis of said collar, making possible the detention and extraction of magnetically susceptible luminal contents.

9. The perivascular jacket according to claim 6, wherein the vascular valve comprises a layer of radiation shielding material in concentric relation to a longitudinal axis of said collar, said radiation shield layer interposed between said outer shell and said cushioning layer,
wherein said radiation shield layer is interrupted to accommodate the opening and closing of said collar, said radiation shield layer serving to allow the passage of low to moderate radiation dose rate radionuclides and radioactive isotopes through said collar and preceding components preceding said collar without causing radiation injury to surrounding tissue.

10. The perivascular jacket according to claim 7, wherein the vascular valve comprises a layer of radiation shielding material in concentric relation to a longitudinal axis of said collar, said radiation shield layer situated along the internal surface of said outer shell to surround said magnet layer,
wherein said radiation shield layer is interrupted to accommodate the opening and closing of said collar, said radiation shield layer serving to allow the passage of low to moderate radiation dose rate radionuclides and radioactive isotopes through said collar and preceding components preceding said collar without causing radiation injury to surrounding tissue.

11. The perivascular jacket according to claim 6, wherein said side tube is entered by a catheteric side tube subsidiary to said side tube, said catheteric side tube allowing the directly targeted delivery of fluid drugs, medical solutions, and tubing maintenance solutions into said side tube, collar, and lumen of said tubular anatomical structure.

12. An automatic homeostasis stabilizer and ambulatory prosthetic disorder response system comprising a plurality of vascular diversion jackets and pumps supplying fluid medicinals to each respective diversion jacket, wherein the pumps are controlled according to a prescription-program by a microcontroller such that:
a plurality of physiological parameter sensors comprising subordinate nodes as negative feedback loops implanted at different locations in the body, the subordinate nodes being configured in a hierarchical control system to said microcontroller and configured to send outputs to said microcontroller such that the microcontroller is a master node, wherein each of the outputs represent feedback,
wherein, when a respective output signals an out-of-range condition to the microcontroller, the microcontroller is configured to respond by causing a respective pump to index to and release a medication prescribed for the respective subordinate node in a dose proportional to the out-of-range output signal received,
wherein, as the master node, the microcontroller governs the discharge of the prescription-program, including dispensing the medication through each subordinate node in a coordinated manner as governed by the prescription-program so that dosing among the subordinate nodes is interrelated to attain the highest possible overall homeostasis such that the system is able to treat comorbid conditions affecting different organ systems in a coordinated manner.

14. The automatic homeostasis stabilizer and ambulatory prosthetic disorder response system of claim 12, wherein the automatic homeostasis stabilizer and ambulatory prosthetic disorder response system is radiation shielded so as to make possible direct administration of radioactive drugs or nonradioactive drugs.

15. A perivascular jacket for encircling a native vessel, said perivascular jacket having bonded to it a tubular outlet passageway defining a lumen continuous with that of said native vessel,
wherein said tubular outlet passageway extends perpendicularly from said perivascular jacket such that said perivascular jacket can be positioned selectively at any anatomically available level and rotational angle,
wherein a valve is disposed within the tubular outlet passageway, said valve comprising a disk or cone perpendicular to a direction of flow through said native vessel, thereby serving as a local regulator of flow pressure, the disk or cone comprising an arm extending a controllable distance into a lumen of said native vessel,
wherein a distal end of the arm comprises an upturned distal edge, thereby serving as a valve to draw away or modulate the flow of a volume through said native vessel into said tubular outlet passageway for discharge out of the perivascular jacket through said tubular outlet passageway.

16. The perivascular jacket according to claim 15, wherein extension and retraction of said disk or cone is driven by a servomotor configured to provide continuously variable control of the extension and retraction of said disk or cone.

17. The perivascular jacket according to claim 15, wherein extension and retraction of said disk or cone is driven by a solenoid.
